(19) United States
(12) Patent Application Publication
(10) Pub. No.: US 2025/0171852 A1
(43) Pub. Date: May 29, 2025

(54) METHOD FOR DETERMINING THE VIRAL OR BACTERIAL NATURE OF AN INFECTION

(71) Applicant: BIOMERIEUX, Marcy L'etoile (FR)

(72) Inventors: Marine MOMMERT, Lyon (FR); Guy ORIOL, Saint-Chamond (FR); Karen BRENGEL-PESCE, Les Abrets En Dauphine (FR); Audrey GUICHARD, Moidieu-Detourbe (FR)

(73) Assignee: BIOMERIEUX, Marcy L'etoile (FR)

(21) Appl. No.: 19/103,141

(22) PCT Filed: Aug. 10, 2023
(86) PCT No.: PCT/EP2023/072136

§ 371 Date: Feb. 11, 2025

(30) Foreign Application Priority Data
Aug. 12, 2022 (FR) FR2208298
Nov. 3, 2022 (EP) 22205313.4

(51) Int. Cl. C12Q 1/6883; C12N 15/10; C12Q 1/6809; C12Q 1/686

(52) U.S. Cl. CPC C12Q 1/6883; C12N 15/1096; C12Q 1/6809; C12Q 1/686; C12Q 2600/158

(57) ABSTRACT

The present invention relates to in vitro or ex vivo methods and kits for determining the viral or bacterial nature of an infection on the basis of a biological sample of a patient by identifying the change in the level of expression of a plurality of biomarkers. In particular, the method includes the steps of (a) measuring the expression of at least one viral target gene and of at least one bacterial target gene chosen from among OLAH, FAM20A, IL1R2, MMP8, RETN and SLC1A2; (b) comparing the expressions measured in step (a) with reference expression values for these target genes; and (c) on the basis of the results of the comparison, concluding whether the infection is viral or bacterial in nature.

### TECHNICAL FIELD

The present invention relates to the technical field of methods and kits for in vitro diagnostics. In particular, a subject of the invention is methods and kits for determining the nature of an infection, i.e. viral or bacterial, from a biological sample from a subject, by identifying the variation in the level of expression of several biomarkers.

### PRIOR ART

Viruses and bacteria interact with different recognition receptors on the surface of leukocytes present in circulating blood. This interaction brings about host-specific transcriptional events that regulate the immune response (Takeuchi et al. 2010). Consequently, differential activation of host transcriptional programs generates unique transcriptomic signatures that make it possible to distinguish viral from bacterial causes.

In this context, analysis of the host transcriptomic profile can provide an indirect approach to detecting the nature of an infection, thus complementing direct approaches such as culturing or nucleic acid amplification (Ramilo et al. 2009). This also represents a particular interest, since viral infections are frequently the cause of fever with no apparent source, notably in young children.

Furthermore, considering the common practice in hospitals of routinely administering antibiotics to febrile patients as a preventive measure until the results of culture tests have been obtained, this unfortunately means that many viral infections are mistakenly treated with antimicrobial drugs.

Furthermore, it has been shown that excessive administration of antibiotics leads to an increase in bacterial resistance, not only on an individual level but also on a more global one. Consequently, the misuse and overuse of available antibiotics actively contribute to the development of antimicrobial resistance (Fauci et al. 2014). What is more, due to difficulties in distinguishing between viral, bacterial or non-infectious etiologies, a certain number of patients are inappropriately treated with antibiotics at high levels.

An early distinction between patients with a viral infection and those with a bacterial infection could thus allow more precise and refined management of said patients, while at the same time significantly reducing unnecessary antibiotic use.

In recent years, there has been growing interest in the discovery of biomarkers that are capable of distinguishing, on the basis of whole blood gene expression, between viral and bacterial infections having similar initial clinical phenotypes. Authors have described, for example, that the transcriptional profiles of febrile viral-positive children and febrile children with acute bacterial infection differ from the profiles of febrile viral-positive and negative children (Hu et al. 2013).

Increasing efforts are thus being made to develop host biomarkers that allow viral infections to be distinguished from bacterial infections, notably in the case of febrile children (D. Brown et al. 2016).

This interest stems not only from the need to distinguish life-threatening bacterial infections from viral ones, but also to avoid the unnecessary prescription of empirical antibiotic therapy, regardless of the severity of the infection.

As mentioned previously, the over-consumption of antibiotics worldwide is accelerating antimicrobial resistance (AMR), which is recognized by the World Health Organization as one of the greatest threats to human health in the coming years.

For example, WO 2018/011316 describes a method for identifying a subject presenting with a bacterial infection. In particular, the method consists in detecting in an mRNA sample the modulation of the expression of two to ten genes chosen from the following gene signature: IFI44L, FAM89A, IFI27L, IFTI1, RSAD2, IFIT3, OTOF, IFIT2, EPSTI1, SERPING1, OAS1, IFI6, HLA-DRB6, HBZ, HS.386275, EIF2AK2, IFIT1L, FCER1A, C21ORF7, GYPE, GYPB, HBM, EIF1AY, LOC649143, HBD, FBX07, KCNMA1, MERTK, EBI3, UPB1, EMR1, PTPN20, TMEM119, SLPI, S100P and PI3.

Discrimination between viral and bacterial infections based on a transcriptomic signature has also been described by other authors. In particular, from an independent cohort of 623 patients, who presented with a bacterial infection, a viral infection, a co-infection or a non-infectious disease, a 45 mRNA signature was identified to discriminate between a viral or bacterial infection (E. L. Tsalik et al., 2021).

However, further efforts are needed to evaluate the accuracy and diagnostic utility of biomarkers, notably transcriptomic biomarkers, before they can be transformed into clinically applicable tests to determine the viral or bacterial origin of an infection.

Thus, although solutions exist, there is still a need to develop novel transcriptomic signatures allowing the nature of an infection to be identified, and thus discriminating between viral and bacterial etiologies. This is of particular importance in order to improve patient management, notably in the case of febrile children, to reduce the inappropriate use of antibiotics and to contribute toward combating the development of antibiotic resistance.

### SUMMARY

The present invention is based on the identification of transcriptomic signatures associated with viral and bacterial infections. The method according to the invention allows the nature of an infection to be identified in a subject diagnosed as having, or suspected of having, an infection, so as to select the most suitable treatment. Entirely advantageously, the method according to the invention makes it possible to rule out the presence of a bacterial infection and to limit the inappropriate use of antibiotics.

Thus, a first aspect of the invention concerns an in vitro or ex vivo method for determining the nature of an infection in a subject by measuring, from a biological sample of said subject, the variation in the level of expression of at least one target viral gene and at least one target bacterial gene chosen from OLAH, FAM20A, IL1R2, MMP8, RETN and SLC1A2.

The method according to the invention, using a biological sample from a subject who is infected or liable to be infected, thus comprises the following steps:
(a) measuring the expression of at least one target viral gene and at least one target bacterial gene chosen from the target bacterial genes OLAH, FAM20A, IL1R2, MMP8, RETN and SLC1A2,
(b) comparing the expressions measured in step (a) with predetermined reference expression values of said target genes, and
(c) concluding as to the viral or bacterial nature of the infection on the basis of the comparison results.

The advantages of the method according to the invention are numerous, notably:
(i) accurately differentiating the viral or bacterial nature of the infection with a high level of performance, with the majority of the biomarker combinations having a performance in terms of area under the curve (AUC) of at least 0.85, or sometimes even at least 0.90,
(ii) ruling out the presence of a bacterial infection,
(iii) rapidly identifying significant variations in the level of expression of genes, notably by the use of automated systems or detection kits according to the invention,
(iv) avoiding the identification of false positives corresponding to the presence of non-pathogenic bacteria or viruses naturally present in subjects,
(v) allowing identification of the nature of the infection when the pathogen is unidentifiable or is inaccessible. Specifically, the present method does not require direct access to the pathogen, only a biological sample from the subject.

After extensive research, the inventors have thus, to their credit, identified a transcriptomic signature whose expression is characteristic of the viral or bacterial nature of the infection, and used the measurement of variation of this expression to identify the source of the infection. This is all the more noteworthy since it is particularly difficult to identify effective target bacterial genes which, when combined with target viral genes, notably allow the presence of a bacterial infection to be ruled out.

Identification of the nature of an infection is of certain interest, notably allowing the clinician to be supported in a more suitable and personalized treatment, notably to avoid the unnecessary prescription of antibiotics when the infection is viral or when the presence of a bacterial infection is ruled out.

Preferably, measurement step (a) comprises or consists in measuring at least one target viral gene chosen from host viral genes involved in the pro-inflammatory cytokine pathway, the interferon pathway, the Toll-like receptor (TLR) signaling pathway, in the RIG-I-like receptor (RLR) signaling pathway and in the Major Histocompatibility Complex (MHC) class II-mediated antigen presentation pathway, and at least one target bacterial gene chosen from OLAH, FAM20A, IL1R2, MMP8, RETN and SLC1A2.

Preferably, measurement step (a) comprises or consists in measuring at least one target viral gene chosen from host viral genes involved in the interferon pathway and the MHC class II-mediated antigen presentation pathway, and at least one target bacterial gene chosen from OLAH, FAM20A, IL1R2, MMP8, RETN and SLC1A2.

Preferably, measurement step (a) comprises or consists in measuring at least one target viral gene chosen from the host viral genes involved in the interferon pathway, and in particular, said genes are chosen from the target viral genes IFI27, IFN-α, IFN-β, IFN-ε, IFN-κ, IFN-ω, IFN-γ, IFNL1, IFNL2, IFNL3, ADAR1, IFIT1, IFIT2, IFIT3, IFIT5, IFI44L, ISG15, ISG20, MDA5, OAS1, OAS2, OAS3, OASL, PARP12, PKR, RIG-I, RNaseL, TRIM25, ZAP, ZCCHC3, ZNFX1, RBBP6, SHFL, TRIM22, TRIM25, TRIM32, TRIM69, Viperin, HERC1, HERC2, HERC3, HERC4, HERC5, HERC6 and combinations thereof, and at least one target bacterial gene chosen from OLAH, FAM20A, IL1R2, MMP8, RETN and SLC1A2.

Preferably, measurement step (a) comprises or consists in measuring at least one target viral gene chosen from RSAD2, IFI27, OAS1, IFIT1, IFI44L, SIGLEC1, ISG15 and HERC6, and at least one target bacterial gene chosen from OLAH, FAM20A, IL1R2, MMP8, RETN and SLC1A2.

Preferably, measurement step (a) comprises or consists in measuring at least one target viral gene chosen from RSAD2, IFI27, OAS1, IFIT1, IFI44L, SIGLEC1, ISG15 and HERC6, and the target bacterial gene FAM20A and optionally at least one other target bacterial gene chosen from OLAH, IL1R2, MMP8, RETN and SLC1A2.

Preferably, measurement step (a) comprises or consists in measuring at least one target viral gene chosen from ISG15 and OAS1, and at least one target bacterial gene chosen from OLAH, FAM20A, IL1R2, MMP8, RETN and SLC1A2.

Preferably, measurement step (a) comprises or consists in measuring at least one target viral gene chosen from SIGLEC1, ISG15 and HERC6, and at least one target bacterial gene chosen from OLAH, FAM20A, IL1R2, MMP8, RETN and SLC1A2. More preferably, measurement step (a) comprises or consists in measuring the target viral genes SIGLEC1, ISG15 and HERC6, and at least one target bacterial gene chosen from OLAH, FAM20A, IL1R2, MMP8, RETN and SLC1A2.

Preferably, measurement step (a) comprises or consists in measuring at least one target viral gene chosen from RSAD2, IFI27, OAS1, IFIT1, IFI44L, SIGLEC1, ISG15 and HERC6, and at least one target bacterial gene chosen from OLAH, FAM20A, IL1R2, MMP8, RETN and SLC1A2.

Preferably, measurement step (a) comprises or consists in measuring at least one target viral gene chosen from SIGLEC1, ISG15 and HERC6, and at least one target bacterial gene chosen from OLAH, FAM20A, IL1R2, MMP8, RETN and SLC1A2.

Preferably, step (a) comprises or consists in measuring at least one target viral gene chosen from IFI27, SIGLEC1, ISG15, HERC6, RSAD2, OAS1, IFIT1 and IFI44L and at least one target bacterial gene chosen from OLAH, FAM20A, IL1R2, MMP8, RETN and SLC1A2.

Preferably, step (a) comprises measuring the expression of the target viral gene IFI27 and of the target bacterial gene OLAH, and more preferably also measuring the expression of the target bacterial gene FAM20A.

Preferably, measurement step (a) comprises or consists in measuring the expression of two target viral genes chosen from IFI27, SIGLEC1, ISG15, HERC6, RSAD2, OAS1, IFIT1 and IFI44L and of two target bacterial genes chosen from OLAH, FAM20A, IL1R2, MMP8, RETN and SLC1A2, preferably chosen from OLAH, FAM20A, IL1R2 and MMP8.

Preferably, measurement step (a) comprises or consists in measuring the expression of two to five target viral genes chosen from SIGLEC1, ISG15, HERC6, RSAD2, IFI27, OAS1, IFIT1 and IFI44L and of three or four target bacterial genes chosen from OLAH, FAM20A, IL1R2, MMP8, RETN and SLC1A2, preferably chosen from OLAH, FAM20A, IL1R2 and MMP8.

Preferably, measurement step (a) comprises or consists in measuring the expression of the target viral gene IFI27, and optionally of at least one other viral gene chosen from SIGLEC1, ISG15, HERC6, RSAD2, OAS1, IFIT1 and IFI44L, and of at least two target bacterial genes chosen from OLAH, FAM20A, IL1R2, MMP8, RETN and SLC1A2, preferably chosen from OLAH, FAM20A, IL1R2 and MMP8.

Preferably, step (a) comprises or consists in measuring at least one target viral gene chosen from SIGLEC1, ISG15 and HERC6, and at least one target bacterial gene chosen from OLAH, FAM20A, IL1R2, MMP8, RETN and SLC1A2, preferably from IL1R2, OLAH and FAM20A.

Preferably, step (a) comprises or consists in measuring at least one target viral gene chosen from IFI27, SIGLEC1, ISG15, HERC6, RSAD2, OAS1, IFIT1 and IFI44L, the target bacterial gene OLAH, and at least one other target bacterial gene chosen from FAM20A, IL1R2, MMP8, RETN and SLC1A2, preferably from FAM20A, IL1R2 and MMP8.

Preferably, step (a) comprises or consists in measuring at least one target viral gene chosen from IFI27, SIGLEC1, ISG15, HERC6, RSAD2, OAS1, IFIT1 and IFI44L, the target bacterial gene FAM20A, and at least one other target bacterial gene chosen from OLAH, IL1R2, MMP8, RETN and SLC1A2, preferably from OLAH, IL1R2 and MMP8.

Preferably, step (a) comprises or consists in measuring the expression of the target viral gene IFI27, of another target viral gene chosen from HERC6, SIGLEC1, IFI44L and ISG15, of the target bacterial gene FAM20A and of another target bacterial gene chosen from OLAH and IL1R2.

Preferably, step (a) comprises or consists in measuring the expression of the target viral gene IFI27, of two other target viral genes chosen from SIGLEC1, HERC6, OAS1 and IFIT1, of the target bacterial gene FAM20A and of another target bacterial gene chosen from OLAH, IL1R2 and MMP8.

Preferably, step (a) comprises or consists in measuring the expression of the target viral genes IFI27 and SIGLEC1, of another target viral gene chosen from IFIT1, HERC6, ISG15 and ISG15, of the target bacterial genes FAM20A and MMP8, and of another target bacterial gene chosen from OLAH and IL1R2.

Preferably, step (a) comprises or consists in measuring the expression of the target viral genes IFI27 and SIGLEC1, and of two other target viral genes chosen from IFIT1, OAS1, HERC6 and ISG15, of the target bacterial genes FAM20A and MMP8, and of another target bacterial gene chosen from OLAH and IL1R2.

Preferably, step (a) consists in measuring a combination of target viral and bacterial genes chosen from the combinations of target genes listed in Tables 6 to 12 and which have an AUC of the model for determining the viral or bacterial nature of the infection of at least 0.90.

Preferably, in the method according to the invention, the reference expression value of the target genes corresponds to the respective expression of said target genes in a reference biological sample obtained from a subject having a bacterial infection. Thus, an infection of viral nature may be concluded when the comparison of the level of expression of the target genes at the mRNA transcript level relative to the respective reference values, highlights at least one variation in expression chosen from:
an over-expression of SIGLEC1,
an over-expression of ISG15,
an over-expression of HERC6,
an over-expression of RSAD2,
an over-expression of IFI27,
an over-expression of OAS1,
an over-expression of IFIT1, and
an over-expression of IFI44L,
   and at least one other expression variation chosen from:
an under-expression of SLC1A2,
an under-expression of IL1R2,
an under-expression of FAM20A,
an under-expression of OLAH,
an under-expression of RETN, and
an under-expression of MMP8.

More preferably, in the method according to the invention, the reference expression value of said target genes corresponds to the respective expression of said target genes in a reference biological sample obtained from a subject having a viral infection. Thus, it can be concluded that the infection is bacterial in nature when the results of the target gene expression comparison reveal at least two variations chosen from the following:
an under-expression of SIGLEC1,
an under-expression of ISG15,
an under-expression of HERC6,
an under-expression of RSAD2,
an under-expression of IFI27,
an under-expression of OAS1,
an under-expression of IFIT1, and
an under-expression of IFI44L,
   and at least one other expression variation chosen from:
an over-expression of SLC1A2,
an over-expression of IL1R2,
an over-expression of FAM20A,
an over-expression of OLAH,
an over-expression of RETN, and
an over-expression of MMP8.

Preferably, the method according to the invention also comprises a step (a′) of measuring the expression of at least one additional target gene chosen from PI3, EBI3, ADGRE1 and S100P, a step (b′) of comparing the expressions measured in step (a′) with reference expression values of said additional target genes, and a step (c′) of concluding whether the infection is viral or bacterial on the basis of the comparative results.

Preferably, in the method according to the invention, the target gene expression is measured at the mRNA level.

Preferably, in the method according to the invention, the expression variation is measured by amplification via RT-PCR, preferably quantitative RT-PCR, or nested PCR.

Preferably, in the method according to the invention, the expression is normalized relative to the expression of one or more housekeeping genes chosen from DECR1, HPRT1, PPIB, GAPDH, and ACTB.

Preferably, in the method according to the invention, the subject is a child, preferably a child less than 4 years old, and more preferably a child less than 4 years old.

Preferably, in the method according to the invention, the biological sample is a blood sample, preferably a whole blood sample.

The invention also relates to a kit for in vitro or ex vivo measurement of the expression of at least one target viral gene chosen from SIGLEC1, ISG15, HERC6, RSAD2, IFI27, OAS1, IFIT1 and IFI44L, and at least one target bacterial gene chosen from OLAH, FAM20A, IL1R2, MMP8, RETN and SLC1A2, said kit comprising reagents specific for the expression products of said target genes, said reagents preferably being primers or probes. The kit is particularly suitable for performing the method according to the invention, and thus enables the viral or bacterial nature of an infection to be determined from a biological sample of a subject.

### BRIEF DESCRIPTION OF THE FIGURES

FIG. 1 represents box plots of expression of the target genes SIGLEC1, ISG15, HERC6, SLC1A2, IL1R2, FAM20A, OLAH, RETN and MMP8 obtained from raw RT-PCR values normalized with the housekeeping genes DECR1, HPRT1 and PPIB; A: Bacterial infection (n=68); B: Viral infection (n=123).

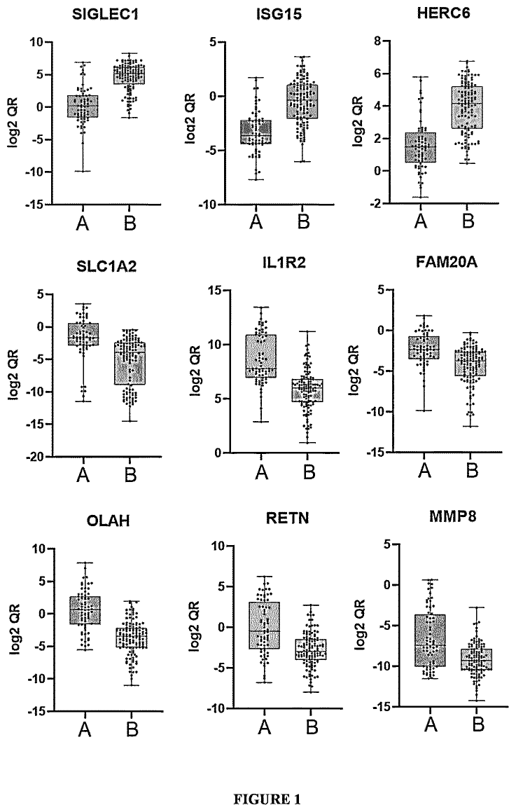

FIG. 2 represents box plots of expression of target genes RSAD2, IFI27, OAS1, IFIT1 and IFI44L obtained from raw RT-PCR values normalized with the housekeeping genes DECR1, HPRT1 and PPIB; A: Bacterial infection (n=68); B: Viral infection (n=123).

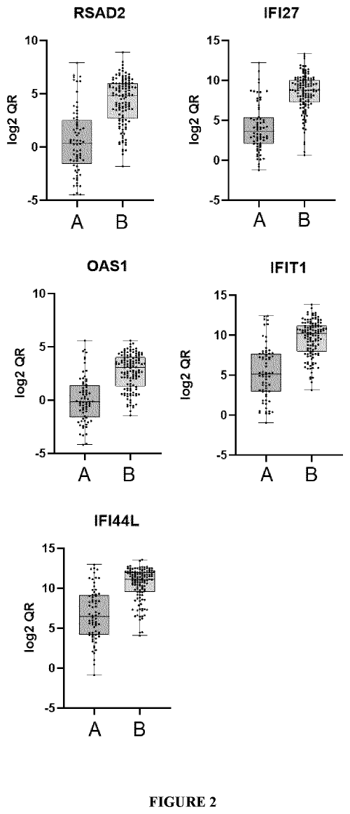

### DETAILLED DESCRIPTION

The transcriptome is the set of RNAs resulting from the transcription of the genome. Transcriptomic analysis can characterize the entire or partial transcriptome of a particular tissue or cell type, or compare transcriptomes between different experimental or clinical conditions. Pathogens such as viruses and bacteria interact in the host with different recognition receptors on the surface of leukocytes present in the circulating blood. As mentioned previously, this interaction entails specific transcriptional events that regulate the immune response, thus generating specific transcriptomic signatures.

Transcriptomic signatures may thus be decision-support tools based on analysis of previously selected gene transcripts.

A first subject of the invention relates to an in vitro or ex vivo method for determining the viral or bacterial nature of an infection in a subject. More particularly, the inventors have identified that the demonstration, from a biological sample of a subject who is infected or liable to be infected, of the variation in the level of expression of several target genes enables the viral or bacterial nature of the infection to be characterized. In other words, the method according to the invention enables the presence of a bacterial infection to be ruled out.

Thus, the method according to the invention comprises the following steps of:
(a) measuring, from the biological sample of an infected subject, the expression of at least one target viral gene and of at least one target bacterial gene chosen from the target bacterial genes OLAH, FAM20A, IL1R2, MMP8, RETN and SLC1A2,
(b) comparing the expressions measured in step (a) with predetermined reference expression values of said target genes, and
(c) concluding as to the viral or bacterial nature of the infection of said subject on the basis of the comparison results.

The expression “target viral gene” refers to a host gene whose expression is deregulated in the presence of a viral infection, i.e. a gene whose expression is significantly increased or reduced in the presence of said infection.

The expression “target bacterial gene” refers to a host gene whose expression is deregulated in the presence of a bacterial infection, i.e. a gene whose expression is significantly increased or reduced in the presence of said infection.

For the purposes of the present description, the expression “target gene” refers indifferently to a target viral gene or a target bacterial gene, unless the context allows it to be clearly identified as a target viral gene or a target bacterial gene.

The terms “biomarker” and “marker” refer to a biological feature that can be objectively measured and which is indicative of normal or pathological biological processes following the presence of an infection. Thus, for the purposes of the present invention, biomarkers are target viral genes and target bacterial genes, and most particularly the transcripts of said target genes.

Throughout the present description, when reference is made to measuring the expression of “at least one target bacterial gene chosen from OLAH, FAM20A, IL1R2, MMP8, RETN and SLC1A2”, this includes the possibility of measuring the expression of several target genes, thus de facto encompassing all possible combinations of 2 to 6 target bacterial genes without it being necessary to give an exhaustive list. This applies to all the lists of target genes introduced in the present description by the term “at least”, and thus also relates to the measurement of target viral gene expression.

The expression “infected subject” refers to a subject having an infection, in other words, a subject having been diagnosed as having an infection. Diagnosis of the presence of infection may be made by any method known to a person skilled in the art for arriving at such a conclusion. The diagnosis may thus be made by a molecular method, such as a procalcitonin (PCT) protein assay, or directly by the clinician on the basis of the subject's clinical symptom(s) or sign(s).

The clinical symptoms or signs associated with the presence of an infection are also well known to those skilled in the art, and mention may be made by way of example of headaches, pain in a specific part of the body (the abdomen, for example), fever (>38° C.) with or without chills, a body temperature below 35.5° C., a respiratory symptom chosen from the group comprising coughing, expectoration, dyspnoea, tachypnoea and pleuritic pain; a finding on auscultation, nausea with or without vomiting, a systolic blood pressure greater than 90 mmHg, a heart rate greater than 120 beats/min, a symptom of infection of an organ or organ system chosen from the group consisting of respiratory tract infections, digestive tract infections, vaginal infections, meningitis, septicemia, erysipelas, peritonitis, cholangitis, cholecystitis and osteomyelitis.

Consequently, the method according to the invention enables the nature of the infection of said subject to be identified.

The expression “nature of the infection” refers solely to the etiology of said infection, i.e. a viral infection or a bacterial infection. The method according to the invention thus enables a viral infection to be distinguished from a bacterial infection, or vice versa. In other words, the method according to the invention enables the presence of a bacterial infection to be ruled out.

For the purposes of the present description, the term “subject” denotes a human being, and the subject is preferably a patient. By definition, the patient is a person who has been in contact with a healthcare professional, notably a doctor, or a medical structure or a healthcare facility.

The target genes involved in the method according to the invention are well known to those skilled in the art, but the inventors have, to their credit, identified that transcriptomic signatures based on the variation in expression of said genes could effectively determine the viral or bacterial nature of an infection.

Specifically, a person skilled in the art is able to identify the target viral genes for implementing the method according to the invention. Similarly, the chromosomal positions of said target genes are accessible in public databases, for instance in the Ensembl database (GRCh38/hg38 assembly).

Thus, any target viral gene whose expression is significantly increased or reduced in the presence of an infection may be used in the method according to the invention. Such genes are known to those skilled in the art.

Consequently, the target viral genes may be chosen from genes of the interferon pathway, the pro-inflammatory cytokine pathway, the Toll-like receptor (TLR) signaling pathway, the RIG-I-like receptor (RLR) signaling pathway, or the MHC class II-mediated antigen presentation pathway.

The target viral genes of the interferon pathway, notably interferon-stimulated genes, known as ISG genes, are well known to those skilled in the art (Schneider et al., 2014; Yang et al. 2020). As examples of these genes, mention may notably be made of IFN-α, IFN-β, IFN-ε, IFN-κ, IFN-ω, IFN-γ, IFNL1, IFNL2, IFNL3, ADAR1, IFIT1, IFIT2, IFIT3, IFIT5, IFI27, IFI44L, ISG15, ISG20, MDA5, OAS1, OAS2, OAS3, OASL, PARP12, PKR, RIG-I, RNaseL, RSAD2, RBBP6, SIGLEC1, SHFL, TRIM22, TRIM25, TRIM32, TRIM69, Viperin, ZAP, ZCCHC3 and ZNFX1.

The target viral genes of the pro-inflammatory cytokine pathway are also well known to those skilled in the art (Mogensen et al. 2001). As examples of these genes, mention may notably be made of TNFα, IL-1β, IL-6, IL-1Ra, IL-8, GM-CSF, MIP-1α, MIP-1β, IL-10, IL-1α, TGF-β, TGF-β1, IL-2, IL-4, IL-13, IL-15, IL-3, IL-5, IP10 and SCM-1.

Thus, according to a particular embodiment, measurement step (a) comprises or consists in measuring at least one target viral gene chosen from host viral genes involved in the pro-inflammatory cytokine pathway, and in particular chosen from the target viral genes TNFα, IL-1β, IL-6, IL-1Ra, IL-8, GM-CSF, MIP-1α, MIP-1β, IL-10, IL-1α, TGF-β, TGF-β1, IL-2, IL-4, IL-13, IL-15, IL-3, IL-5, IP10, SCM-1 and combinations thereof, and at least one target bacterial gene chosen from OLAH, FAM20A, IL1R2, MMP8, RETN and SLC1A2.

The target viral genes of the Toll-like receptor pathway are also well known to those skilled in the art (Kawasaki et al., 2014) and notably comprise native TLR genes, TLR co-receptor genes, TLR regulator genes, TLR signaling inhibitor genes, TLR adaptor genes and TLR signaling genes.

As examples of native TLR genes, mention will notably be made of the genes TLR3, TLR7, TLR8 and TLR9.

As examples of TLR co-receptor genes, mention will notably be made of the genes CD14, LBP, MD1 and MD2.

As examples of TLR-regulating genes, mention will notably be made of the genes CD300LF, GRP94, PRAT4A, PRAT4B and UNC93B1.

As examples of TLR signaling-inhibitor genes, mention will notably be made of the genes ATF3, AXL, BAMBI, BCL3, BPI, CENTB1, DAK, FLII, HSP10, IRAKM(IRAK3), LGP2(DHX58), MSK1, MSK2, NLRP12(NALP12), NFKBIA, NFKBIB, NFKBIE, PECAM1, PIN1, PPP3CA, PPP3CB, PPP3R1, PTPN6, RNF125, RP105, SHIP1, SIGIRR, SIKE, TNFAIP3, TNIP1, TNIP2, TNIP3, TOLLIP, TRAFD1, TRIAD3, TYRO3 and ZCCHC11.

As examples of TLR adaptor genes, mention will notably be made of the genes MyD88, RAC1, SARM1, TANK, TIRAP, TRAM(TICAM2) and TRIF(TICAM1).

As examples of TLR signaling genes, mention will notably be made of the genes AAMP, ACT1(TRAF3IP2), AGER, AKT1, BECN1, BTK, CARD6, CARD11, DDX3X, FADD, IKKα, IKKβ, IKKε, IPS1, IRAK1, IRAK4, IRF1, IRF3, IRF5, IRF7, IRF8, IRF9, ITCH, LRRC59, LRRFIP2, MAP2K2, MAP3K7(TAK1), MAPK1, MDA5(IFIH1), NAIP5, NAP1, NEMO, NIBP, OTUD5, PKR, PRKRA, RIG-1, RIPK1, RIPK2, RIPK3, STAP2, SUGT1, SYK, TAB1, TAB2, TAB3, TBK1, TBKBP1, TIFA, TRADD, TRAF3, TRAF6, TRIL and WDFY1.

Thus, according to a particular embodiment, measurement step (a) comprises or consists in measuring at least one target viral gene chosen from host viral genes involved in the TLR receptor signaling pathway, and in particular chosen from TLR3, TLR7, TLR8, TLR9, CD14, LBP, MD1, MD2, CD300LF, GRP94, PRAT4A, PRAT4B, UNC93B1 ATF3, AXL, BAMBI, BCL3, BPI, CENTB1, DAK, FLII, HSP10, IRAKM(IRAK3), LGP2(DHX58), MSK1, MSK2, NLRP12(NALP12), NFKBIA, NFKBIB, NFKBIE, PECAM1, PIN1, PPP3CA, PPP3CB, PPP3R1, PTPN6, RNF125, RP105, SHIP1, SIGIRR, SIKE, TNFAIP3, TNIP1, TNIP2, TNIP3, TOLLIP, TRAFD1, TRIAD3, TYRO3, ZCCHC11, MyD88, RAC1, SARM1, TANK, TIRAP, TRAM(TICAM2), TRIF(TICAM1), AAMP, ACT1(TRAF3IP2), AGER, AKT1, BECN1, BTK, CARD6, CARD11, DDX3X, FADD, IKKα, IKKβ, IKK&, IPS1, IRAK1, IRAK4, IRF1, IRF3, IRF5, IRF7, IRF8, IRF9, ITCH, LRRC59, LRRFIP2, MAP2K2, MAP3K7(TAK1), MAPK1, MDA5(IFIH1), NAIP5, NAP1, NEMO, NIBP, OTUD5, PKR, PRKRA, RIG-1, RIPK1, RIPK2, RIPK3, STAP2, SUGT1, SYK, TAB1, TAB2, TAB3, TBK1, TBKBP1, TIFA, TRADD, TRAF3, TRAF6, TRIL, WDFY1 and combinations thereof, and at least one target bacterial gene chosen from OLAH, FAM20A, IL1R2, MMP8, RETN and SLC1A2.

The target viral genes of the RIG-I-like receptor signaling pathway are also known to those skilled in the art. Examples that may be mentioned include the genes LGP2(DHX58), MDA5(IFIH1), RIG-I(DDX58), TRAFD1, CYLD, DAK, DDX60, MFN2, MUL1, OTUD5, PIN1, RNF125, SIKE, TNFAIP3, IPS1, IPS1-HA, PARP13, STING, TOMM70A, TRIM13, TRIM26, TRIM32 and ZDHHC1.

Thus, according to a particular embodiment, measurement step (a) comprises or consists in measuring at least one target viral gene chosen from host viral genes involved in the RLR receptor signaling pathway, and in particular, said genes are chosen from the target viral genes LGP2(DHX58), MDA5(IFIH1), RIG-I(DDX58), TRAFD1, CYLD, DAK, DDX60, MFN2, MUL1, OTUD5, PIN1, RNF125, SIKE, TNFAIP3, IPS1, IPS1-HA, PARP13, STING, TOMM70A, TRIM13, TRIM26, TRIM32, ZDHHC1 and combinations thereof, and at least one target bacterial gene chosen from OLAH, FAM20A, IL1R2, MMP8, RETN and SLC1A2.

Finally, the target viral genes of the MHC class II-mediated antigen presentation pathway are also well known to those skilled in the art. By way of example, mention may be made of target genes of the ubiquitin ligase family, notably HERC1, HERC2, HERC3, HERC4, HERC5 and HERC6 (Hochrainer et al., 2005).

Thus, according to a particular embodiment, the target viral genes whose expression is measured in step (a) are chosen from the target viral genes of the pro-inflammatory cytokine pathway, the interferon pathway, the Toll-like receptor (TLR) signaling pathway, the RIG-I-like receptor (RLR) signaling pathway, and the MHC class II-mediated antigen presentation pathway.

According to a particular embodiment, the target viral genes whose expression is measured in step (a) are chosen from TNFα, IL-1β, IL-6, IL-1Ra, IL-8, GM-CSF, MIP-1α, MIP-1β, IL-10, IL-1α, TGF-β, TGF-β1, IL-2, IL-4, IL-13, IL-15, IL-3, IL-5, IP10, SCM-1, IFN-α, IFN-β, IFN-ε, IFN-κ, IFN-0, IFN-γ, IFNL1, IFNL2, IFNL3, ADAR1, IFIT1, IFIT2, IFIT3, IFIT5, IFI27, IFI44L, ISG15, ISG20, MDA5, OAS1, OAS2, OAS3, OASL, PARP12, PKR, RIG-I, RNaseL, ZAP, ZCCHC3, ZNFX1, RBBP6, SHFL, TRIM22, TRIM25, TRIM32, TRIM69, Viperin, TLR3, TLR7, TLR8, TLR9, CD14, LBP, MD1, MD2, CD300LF, GRP94, PRAT4A, PRAT4B, UNC93B1 ATF3, AXL, BAMBI, BCL3, BPI, CENTB1, DAK, FLII, HSP10, IRAKM(IRAK3), LGP2(DHX58), MSK1, MSK2, NLRP12(NALP12), NFKBIA, NFKBIB, NFKBIE, PECAM1, PIN1, PPP3CA, PPP3CB, PPP3R1, PTPN6, RNF125, RP105, SHIP1, SIGIRR, SIKE, TNFAIP3, TNIP1, TNIP2, TNIP3, TOLLIP, TRAFD1, TRIAD3, TYRO3, ZCCHC11, MyD88, RAC1, SARM1, TANK, TIRAP, TRAM(TICAM2), TRIF(TICAM1), AAMP, ACT1(TRAF3IP2), AGER, AKT1, BECN1, BTK, CARD6, CARD11, DDX3X, FADD, IKKα, IKKβ, IKK&, IPS1, IRAK1, IRAK4, IRF1, IRF3, IRF5, IRF7, IRF8, IRF9, ITCH, LRRC59, LRRFIP2, MAP2K2, MAP3K7(TAK1), MAPK1, MDA5(IFIH1), NAIP5, NAP1, NEMO, NIBP, OTUD5, PKR, PRKRA, RIG-1, RIPK1, RIPK2, RIPK3, STAP2, SUGT1, SYK, TAB1, TAB2, TAB3, TBK1, TBKBP1, TIFA, TRADD, TRAF3, TRAF6, TRIL, WDFY1, LGP2(DHX58), MDA5(IFIH1), RIG-I(DDX58), TRAFD1, CYLD, DAK, DDX60, MFN2, MUL1, OTUD5, PIN1, RNF125, SIKE, TNFAIP3, IPS1, IPS1-HA, PARP13, STING, TOMM70A, TRIM13, TRIM26, TRIM32, ZDHHC1, HERC1, HERC2, HERC3, HERC4, HERC5, HERC6 and combinations thereof.

According to a preferred embodiment, the target viral genes whose expression is measured in step (a) are chosen from the target viral genes of the interferon pathway and the MHC class II-mediated antigen presentation pathway.

According to another preferred embodiment, measurement step (a) comprises or consists in measuring at least one target viral gene chosen from IFN-α, IFN-β, IFN-ε, IFN-κ, IFN-ω, IFN-γ, IFNL1, IFNL2, IFNL3, ADAR1, IFIT1, IFIT2, IFIT3, IFIT5, IFI27, IFI44L, ISG15, ISG20, MDA5, OAS1, OAS2, OAS3, OASL, PARP12, PKR, RIG-I, RNaseL, RSAD2, RBBP6, SIGLEC1, SHFL, TRIM22, TRIM25, TRIM32, TRIM69, Viperin, ZAP, ZCCHC3, ZNFX1, HERC1, HERC2, HERC3, HERC4, HERC5, HERC6 and combinations thereof, and at least one target bacterial gene chosen from OLAH, FAM20A, IL1R2, MMP8, RETN and SLC1A2.

According to another preferred embodiment, measurement step (a) comprises or consists in measuring at least one target viral gene chosen from IFI27, IFN-γ, IFIT1, IFIT2, IFI44L, ISG15, OAS1, OAS2, OAS3, RSAD2, TRIM25, SIGLEC1, TRIM22, TRIM32, HERC5, HERC6 and combinations thereof, and at least one target bacterial gene chosen from OLAH, FAM20A, IL1R2, MMP8, RETN and SLC1A2.

According to another particularly preferred embodiment, measurement step (a) comprises or consists in measuring at least one target viral gene chosen from SIGLEC1, RSAD2, IFI27, OAS1, IFIT1, IFI44L, ISG15 and HERC6, and at least one target bacterial gene chosen from OLAH, FAM20A, IL1R2, MMP8, RETN and SLC1A2.

The chromosomal positions of the target genes according to this preferred embodiment are notably given in Table 1 below:

TABLE 1

| | Chromosomal location | |
|---|---|---|
| Gene | (GRCh38/hg38) | English name |
| SIGLEC1 | Chr20: 3,686,970-3,712,600 | Sialic Acid Binding Ig Like Lectin 1 |
| ISG15 | Chr1: 1,001,138-1,014,540 | ISG15 Ubiquitin Like Modifier |
| HERC6 | Chr4: 88378739-88443097 | HECT And RLD Domain Containing E3 |
| | | Ubiquitin Protein Ligase Family Member 6 |
| SLC1A2 | Chr11: 35251205-35420063 | Solute Carrier Family 1 Member 2 |
| ILIR2 | Chr2: 101991960-102028544 | Interleukin 1 Receptor Type 2 |
| FAM20A | Chr17: 68535113-68601367 | Golgi Associated Secretory Pathway |
| | | Pseudokinase |
| OLAH | Chr10: 15032227-15073853 | Oleoy1-ACP Hydrolase |
| RETN | Chr19: 7669049-7670455 | Resistin |
| MMP8 | Chr11: 102711796-102727050 | Matrix Metallopeptidase 8 |
| RSAD2 | Chr2: 6865557-6898239 | Radical S-Adenosy1 Methionine Domain |
| | | Containing 2 |
| IFI27 | Chr14: 94104836-94116698 | Interferon Alpha Inducible Protein 27 |
| OAS1 | Chr12: 112905856-112933219 | 2′-5′-Oligoadenylate Synthetase 1 |
| IFIT1 | Chr10: 89392546-89406487 | Interferon Induced Protein With |
| | | Tetratricopeptide Repeats 1 |
| IFI44L | Chr1: 78619902-78646145 | Interferon Induced Protein 44 Like |

The SIGLEC1 gene encodes a member of the immunoglobulin superfamily. The encoded protein is a lectin-like adhesion molecule that binds to glycoconjugate ligands on cell surfaces in a sialic acid-dependent manner. It is a type I transmembrane protein expressed only by a subpopulation of macrophages, and is involved in mediating cell-cell interactions.

The ISG15 gene encodes a ubiquitin-like protein that is conjugated to intracellular target proteins upon activation by interferon-alpha and interferon-beta. Several functions have been attributed to the encoded protein, notably chemotactic activity toward neutrophils, direction of target proteins conjugated to intermediate filaments, intercellular signaling and antiviral activity during viral infections.

The HERC6 gene belongs to the HERC family of ubiquitin ligases, all of which contain at least one 350-amino acid HECT domain catalyzing the formation of a thioester with ubiquitin before transferring it to a substrate.

The SLC1A2 gene encodes a member of a family of solute transporter proteins. This membrane-bound protein is the main transporter that removes the excitatory neurotransmitter glutamate from the extracellular space at synapses in the central nervous system. Glutamate clearance is necessary for correct synaptic activation and for preventing neuronal damage due to excessive activation of the glutamate receptors.

The IL1R2 gene encodes a cytokine receptor protein belonging to the interleukin-1 receptor family. This protein binds interleukin alpha (IL1(A), interleukin beta (IL1B) and the type I interleukin 1 receptor (IL1R1/IL1R(A), and acts as a decoy receptor that inhibits the activity of its ligands. Interleukin 4 (IL4) antagonizes the activity of interleukin 1 by inducing the expression and release of this cytokine. This gene and three others form a group of cytokine receptor genes on chromosome 2q12. Alternative splicing gives rise to multiple transcription variants and isoforms of membrane proteins and soluble proteins.

The FAM20A gene encodes a presumably secreted protein that may play a role in hematopoiesis. A mutation at this locus has been associated with amelogenesis imperfecta and gingival hyperplasia syndrome.

The OLAH gene allows dodecanoyl hydrolase activity [acyl-protein bearing]; myristoyl hydrolase activity [acyl-protein bearing]; and palmitoyl hydrolase activity [acyl-protein bearing]. It is involved in the biosynthesis of medium-chain fatty acids.

The RETN gene encodes a protein having an antimicrobial role in the skin via antibacterial activity against Gram-positive and Gram-negative bacteria.

The MMP8 gene encodes a member of the matrix metalloproteinase (MMP) family of proteins. These proteins are involved in the degradation of the extracellular matrix during embryonic development, reproduction and tissue remodeling, and also in pathological processes such as arthritis and metastasis. Proteolysis at different sites on this protein gives rise to several active forms of the enzyme with different N-termini. This protein functions in the degradation of type I, II and III collagens.

The RASAD2 gene encodes an interferon-inducible antiviral protein belonging to the S-adenosyl-L-methionine (SAM) enzyme superfamily, which plays a role in the cellular antiviral response and innate immune signaling.

The antiviral effects result notably from inhibition of viral RNA replication, interference with the secretion pathway, binding to viral proteins and deregulation of cellular lipid metabolism.

The IFI27 gene is notably involved in the metabolic process of cellular proteins, the defense response to other organisms and the extrinsic apoptotic signaling pathway. This gene also acts upstream or in the down-regulation of transcription by RNA polymerase II and the regulation of protein export from the nucleus.

The OAS1 gene encodes a protein that synthesizes 2′,5′-oligoadenylates (2-5As) and plays a key role in the innate cellular antiviral response.

The IFIT1 gene encodes a protein containing tetratricopeptide repeats originally identified as being induced by interferon treatment. The encoded protein can inhibit viral replication and translation initiation.

The IFI44L gene is associated with GTP-binding activity and involved in the viral defense response.

According to the present description, identifying or measuring the level of gene expression consists in highlighting a variation in the transcriptomic expression of said genes, said variation being highlighted relative to a reference expression of said genes. For this reason, the term “transcriptomic signature” will notably be used.

The transcripts of the various target genes that are useful for performing the method according to the present description are also known to those skilled in the art, and their sequences are available in the NCBI or Ensembl databases.

Examples of transcripts are shown in Table 2 hereinbelow for certain preferred target viral and bacterial genes:

TABLE 2

| Gene | Transcribed reference (NCBI database) |
|---|---|
| SIGLEC1 | NM_023068.4; NM_001367089.1 |
| ISG15 | NM_005101.4 |
| HERC6 | NM_017912.4 |
| | NM_001165136.2 |
| SLC1A2 | NM_004171.4; NM_001195728.3; NM_001252652.2 |
| IL1R2 | NM_004633.4; NM_001261419.2 |
| FAM20A | NM_017565.4; NM_001243746.2 |
| OLAH | NM_001039702.3; NM_018324.3 |
| RETN | NM_020415.4; NM_001385725.1; NM_001385726.1; NM_001385727.1; |
| | NM_001193374.2 |
| MMP8 | NM_002424.3; NM_001304442.2; NM_001304441.2 |
| RSAD2 | NM_080657.5; NM_001410702.1 |
| IFI27 | NM_001366994.1; NM_001366993.1; NM_001288952.2; |
| | NM_001130080.3; NM_001288956.2; NM_001288959.2 |
| OAS1 | NM_001320151.2; NM_016816.4; NM_001032409.3; NM_002534.4; |
| | NM_001406027.1; NM_001406030.1; NM_001406025.1; |
| | NM_001406024.1; NM_001406029.1; NM_001406020.1; |
| | NM_001406026.1; NM_001406023.1; NM_001406021.1; |
| | NM_001406022.1 |
| IFIT1 | NM_001270930.2; NM_001270927.2; NM_001270928.2; |
| | NM_001270929.2; NM_001548.5 |
| IFI44L | NM_001375646.1; NM_001375650.1; NM_006820.4; NM_001375647.1; |
| | NM_001375649.1; NM_001375648.1 |

According to a particular embodiment, the expression of the target viral and bacterial genes is measured at the level of mRNA transcripts, and preferably at the level of the transcripts chosen from the transcripts in Table 2.

According to a preferred embodiment, the expression of the target genes is measured at the level of mRNA transcripts, and preferably at the level of transcripts chosen from the transcripts (NCBI database) NM_023068.4, NM_001367089.1, NM_005101.4, NM_017912.4, NM_001165136.2, NM_017565.4, NM_001243746.2, NM_002424.3, NM_001304442.2, NM_001304441.2, NM_001039702.3, NM_018324.3, NM_004633.4, NM_001261419.2, NM_003256.4, NM_004171.4, NM_001195728.3, NM_001252652.2, NM_020415.4, NM_001385725.1, NM_001385726.1, NM_001385727.1, NM_001193374.2 and combinations thereof.

Any method known to those skilled in the art for measuring the level of gene expression, or a variation in gene expression, at the transcriptional level may be used in the context of the method according to the invention.

Thus, the measurement may be performed via a direct method for determining the presence of said transcript in the biological sample, or by indirect detection of the transcript after transformation of the latter into DNA.

Techniques commonly used to simultaneously measure the concentration of a large number of different types of messenger RNA are known to those skilled in the art, and need not be detailed here. By way of example, mention will notably be made of DNA chips, CAGE, SAGE and, more recently, high-throughput RNA sequencing, referred to as RNA-Seq.

According to the present description, gene expression at the transcriptional level may be measured by any known molecular detection method. Thus, the variation in gene expression may be measured by amplification notably via Reverse Transcription-Polymerase Chain Reaction or RT-PCR, by sequencing (preferably by high-throughput sequencing) or by hybridization techniques (for example with hybridization microarrays or by techniques of the NanoString® nCounter® type). All of these methods are also well known to those skilled in the art, and it is not necessary to describe them in detail here.

According to a particular embodiment, the determination of gene expression may be performed in the following manner:
(1) extraction of total RNAs from a blood sample or PBMCs and performing a step of reverse transcription in order to obtain the various complementary DNAs (or cDNA) to the various messenger RNAs initially present in the sample or the PBMCs,
(2) specific amplification of the cDNAs. In this case, the specific reagent used comprises at least one specific amplification primer for the gene. This step can be performed by a PCR-type amplification reaction or by any other suitable amplification technique,
(3) determination of the expression of the gene by quantifying the cDNAs.

According to a preferred embodiment, gene expression is measured by RT-PCR, preferably quantitative or semi-quantitative RT-PCR, or nested PCR, for example using FilmArray® technology (Poritz et al. 2011) or Fluidigm's BiomarkTM platform. According to this embodiment, expression is measured at the level of mRNA transcripts of the target genes.

A person skilled in the art is entirely capable of determining the sequences of the primers or primer pairs required to amplify the transcripts of the target genes, and optionally additional target genes defined later in the description, to determine their respective levels of expression. Specifically, numerous tools are available, for example Geneious or Primer 3, and said sequences can then be adjusted as required by a person skilled in the art.

The measurement of the expression level makes it possible to determine the quantity of transcripts in the biological sample or also to give a value derived therefrom.

According to a particular embodiment, the level of expression of the signature target genes is a derived or normalized value of the amount of transcripts, notably mRNA, of said target genes.

According to a particular embodiment, the gene expression is normalized relative to the expression of one or more housekeeping genes (or reference genes) according to the methods known to those skilled in the art. Thus, the expression is normalized using one or more of the following housekeeping genes: DECR1 (chromosomal location: chr8, 90001352-90053633), HPRT1 (chromosomal location: chrX, 134452842-134520513) and PPIB (chromosomal location: chr15: 64155812-64163205), RPLPO (chromosomal location: chr12, 120196699-120201111), PPIA (chromosomal location: chr7, 44795960-44803117), GLYR1 (chromosomal location: chr16, 4803203-4847288), RANBP3 (chromosomal location: chr19, 5916139-5978140), B2M (chromosomal location: chr15, 44711492-44718145), TBP (chromosomal location: chr6, 170554369-170572859), GAPDH (chromosomal location: chr12, 6534517-6538371) and ACTB (chromosomal location: chr14, 5527148-5530601). The chromosomal locations are given according to GRCh38/hg38. Preferably, the expression is normalized using one or more housekeeping genes chosen from: DECR1, HPRT1, PPIB, GAPDH, ACTB and combinations thereof, more preferably chosen from DECR1, HPRT1, PPIB and combinations thereof.

In such a case, the reference level used is also normalized beforehand, in the same manner. The normalization, whether for the reference level or for the level of transcripts of the biological sample to be tested, is performed before the comparison, notably before the calculation of a relationship between the level of transcripts of said sample to be tested and the reference level. When a threshold value different from a reference level is used to draw a conclusion, this normalization may be taken into account for the choice of the threshold value.

In the event that the level of gene transcripts is normalized relative to the level of transcripts of one or more housekeeping genes, this of course implies that the present method includes determining the level of transcripts of the housekeeping gene(s) used for the normalization.

Generally speaking, according to the method of the present invention, and regardless of the embodiments thereof, the expression level of a target gene (preferably the normalized expression) in the biological sample from the subject is compared to a predetermined expression value of this same gene (preferably the normalized expression) in a reference biological sample. This comparison makes it possible to obtain the variation in the expression of said target gene measured according to the present method.

For a given target gene, the reference expression value corresponds to a level of expression of transcripts of said gene obtained from a reference biological sample obtained from a subject having an infection of a given nature. The reference biological sample is of the same nature as the biological sample to be tested, or at least of a compatible nature, so as to constitute a reference for determining the level of expression of the target genes.

Advantageously, and notably for the embodiments hereinbelow, the reference value for a given gene corresponds to the average level of mRNA transcripts of said gene obtained from reference biological samples from a population of subjects presenting with an infection.

For the purposes of the present invention, the expression “reference value” or “predetermined reference value” is synonymous with the expressions “control value” or “threshold value” and serves as a point of comparison for determining whether the expression level of a target gene is decreased or increased.

According to a particular embodiment, the target gene reference value corresponds to the level of expression of the mRNA transcripts of said genes obtained from a reference biological sample from a subject having a bacterial infection.

According to a particular embodiment, the target gene reference value corresponds to the level of expression of the mRNA transcripts of said genes obtained from a reference biological sample from a subject having a viral infection.

The comparison may be performed by any method known to those skilled in the art, and may, for example, involve calculating a ratio or a difference. Advantageously, in the context of the present method, the comparison(s) and the issuing of a conclusion as to the nature of the infection in the subject from whom the biological sample is taken, is performed via an automated, computer-operated or computer-assisted technique.

Thus, the viral or bacterial nature of the subject's infection is determined when comparisons of the expression levels of the target genes with their respective reference values allow identification of a statistically significant difference, in other words, a variation in said expression level. Consequently, the term “over-expression” is used when significantly increased expression is detected, and conversely, “under-expression” is used when significantly reduced expression is detected.

A person skilled in the art is able to determine the statistical test to be used to determine this reference value with which the expression level of the target genes is to be compared. The exemplary embodiments present one of the possible methods.

According to a first implementation variant of the invention, the reference expression value of each target gene is determined on the basis of a reference biological sample from a pool of biological samples from subjects suffering from a bacterial infection. Preferably, in the method according to the invention, the reference expression value of the target genes corresponds to the respective expression of said target genes in a reference biological sample obtained from a subject having a bacterial infection. Thus, an infection of viral nature may be concluded when the comparison of the level of expression of the target genes at the mRNA transcript level relative to the respective reference values, highlights at least one variation in expression chosen from:
an over-expression of SIGLEC1,
an over-expression of ISG15,
an over-expression of HERC6,
an over-expression of RSAD2,
an over-expression of IFI27,
an over-expression of OAS1,
an over-expression of IFIT1, and
an over-expression of IFI44L,
   and at least one other expression variation chosen from:
an under-expression of SLC1A2,
an under-expression of IL1R2,
an under-expression of FAM20A,
an under-expression of OLAH,
an under-expression of RETN, and
an under-expression of MMP8.

According to a second implementation variant of the invention, the reference value of each target gene is determined on the basis of a reference biological sample from a pool of biological samples from subjects suffering from a viral infection. Thus, it can be concluded that the infection is bacterial in nature when the results of the target gene expression comparison reveal at least two variations chosen from the following:
an under-expression of SIGLEC1,
an under-expression of ISG15,
an under-expression of HERC6,
an under-expression of RSAD2,
an under-expression of IFI27,
an under-expression of OAS1,
an under-expression of IFIT1, and
an under-expression of IFI44L,
   and at least one other expression variation chosen from:
an over-expression of SLC1A2,
an over-expression of IL1R2,
an over-expression of FAM20A,
an over-expression of OLAH,
an over-expression of RETN, and
an over-expression of MMP8.

A person skilled in the art is able to opt for one or other of the variants as a function of the reference biological samples available. The reference biological sample is advantageously a pool of biological samples from subjects having a bacterial infection or a pool of biological samples from subjects having a viral infection.

In all the particular embodiments described hereinbelow, the conclusion as to the nature of the infection is reached by taking into account the over/under-expression of the various target genes detected in the subject's biological sample, and as defined previously in one or other of the two embodiment variants.

According to a particular embodiment, step (a) comprises or consists in measuring the expression of at least one target viral gene chosen from SIGLEC1, ISG15, HERC6, RSAD2, IFI27, OAS1, IFIT1 and IFI44L, and of at least one target bacterial gene chosen from OLAH, FAM20A, IL1R2, MMP8, RETN and SLC1A2.

According to another particular embodiment, step (a) comprises or consists in measuring the expression of a single target viral gene chosen from SIGLEC1, ISG15, HERC6, RSAD2, IFI27, OAS1, IFIT1 and IFI44L, and of two, three, four, five or six target bacterial genes chosen from OLAH, FAM20A, IL1R2, MMP8, RETN and SLC1A2, and preferably of two or three target bacterial genes chosen from OLAH, FAM20A, IL1R2, MMP8, RETN and SLC1A2.

According to another particular embodiment, step (a) comprises or consists in measuring the expression of two target viral genes chosen from SIGLEC1, ISG15, HERC6, RSAD2, IFI27, OAS1, IFIT1 and IFI44L, and of two, three, four, five or six target bacterial genes chosen from OLAH, FAM20A, IL1R2, MMP8, RETN and SLC1A2, and preferably of two or three target bacterial genes chosen from OLAH, FAM20A, IL1R2, MMP8, RETN and SLC1A2,

According to another particular embodiment, step (a) comprises or consists in measuring the expression of three target viral genes chosen from SIGLEC1, ISG15, HERC6, RSAD2, IFI27, OAS1, IFIT1 and IFI44L, and of two, three, four, five or six target bacterial genes chosen from OLAH, FAM20A, IL1R2, MMP8, RETN and SLC1A2.

According to another particular embodiment, step (a) comprises or consists in measuring the expression of four target viral genes chosen from SIGLEC1, ISG15, HERC6, RSAD2, IFI27, OAS1, IFIT1 and IFI44L, and of two, three, four, five or six target bacterial genes chosen from OLAH, FAM20A, IL1R2, MMP8, RETN and SLC1A2.

According to another particular embodiment, step (a) comprises or consists in measuring the expression of five target viral genes chosen from SIGLEC1, ISG15, HERC6, RSAD2, IFI27, OAS1, IFIT1 and IFI44L, and of two, three, four, five or six target bacterial genes chosen from OLAH, FAM20A, IL1R2, MMP8, RETN and SLC1A2.

According to another particular embodiment, step (a) comprises or consists in measuring the expression of six target viral genes chosen from SIGLEC1, ISG15, HERC6, RSAD2, IFI27, OAS1, IFIT1 and IFI44L, and of two, three, four, five or six target bacterial genes chosen from OLAH, FAM20A, IL1R2, MMP8, RETN and SLC1A2.

According to another particular embodiment, step (a) comprises or consists in measuring the expression of seven target viral genes chosen from SIGLEC1, ISG15, HERC6, RSAD2, IFI27, OAS1, IFIT1 and IFI44L, and of two, three, four, five or six target bacterial genes chosen from OLAH, FAM20A, IL1R2, MMP8, RETN and SLC1A2.

According to another particular embodiment, step (a) comprises or consists in measuring the expression of the eight target viral genes SIGLEC1, ISG15, HERC6, RSAD2, IFI27, OAS1, IFIT1 and IFI44L, and of two, three, four, five or six target bacterial genes chosen from OLAH, FAM20A, IL1R2, MMP8, RETN and SLC1A2.

According to another particular embodiment, step (a) comprises or consists in measuring the expression of the target viral gene SIGLEC1 and of one or more target bacterial genes chosen from OLAH, FAM20A, IL1R2, MMP8, RETN and SLC1A2, preferably from SLC1A2, IL1R2, OLAH, FAM20A and RETN, and even more preferably from SLC1A2, IL1R2, OLAH and FAM20A.

According to a preferred variant of this embodiment, step (a) comprises or consists in measuring the expression of the target viral gene SIGLEC1 and of at least three target bacterial genes chosen from SLC1A2, IL1R2, OLAH, FAM20A and RETN.

According to another particular embodiment, step (a) comprises or consists in measuring the expression of the target viral gene ISG15 and of one or more target bacterial genes chosen from, OLAH, FAM20A, IL1R2, MMP8, RETN and SLC1A2, preferably from SLC1A2, IL1R2, OLAH, FAM20A and RETN, and even more preferably from SLC1A2, IL1R2, OLAH and FAM20A.

According to another particular embodiment, the measurement step comprises or consists in measuring the expression of the viral gene HERC6 and of one or more target bacterial genes chosen from OLAH, FAM20A, IL1R2, MMP8, RETN and SLC1A2, preferably from SLC1A2, IL1R2, OLAH, FAM20A and RETN, and more preferably from SLC1A2, IL1R2, OLAH and FAM20A.

According to another particular embodiment, step (a) comprises or consists in measuring the expression of the target viral gene IFIT1 and of one or more target bacterial genes chosen from OLAH, FAM20A, IL1R2, MMP8, RETN and SLC1A2.

According to another particular embodiment, step (a) comprises or consists in measuring the expression of the target viral gene IFI44L and of one or more target bacterial genes chosen from OLAH, FAM20A, IL1R2, MMP8, RETN and SLC1A2.

According to another particular embodiment, step (a) comprises or consists in measuring the expression of the target viral gene RSAD2 and of one or more target bacterial genes chosen from OLAH, FAM20A, IL1R2, MMP8, RETN and SLC1A2.

According to another particular embodiment, step (a) comprises or consists in measuring the expression of the target viral gene OAS1 and of one or more target bacterial genes chosen from OLAH, FAM20A, IL1R2, MMP8, RETN and SLC1A2.

According to a preferred embodiment, step (a) comprises or consists in measuring the expression of the target viral gene IFI27 and of one or more target bacterial genes chosen from OLAH, FAM20A, IL1R2, MMP8, RETN and SLC1A2, and preferably from IL1R2, OLAH, FAM20 and RETN.

According to another preferred embodiment, step (a) comprises or consists in measuring the expression of the target viral gene IFI27, and optionally of at least one other viral gene chosen from SIGLEC1, ISG15, HERC6, RSAD2, OAS1, IFIT1, IFI44L, and of two target bacterial genes chosen from OLAH, FAM20A, IL1R2, MMP8, RETN and SLC1A2, and preferably chosen from OLAH, FAM20A, IL1R2 and MMP8.

According to another preferred embodiment, step (a) comprises or consists in measuring the expression of the target viral gene IFI27, the target bacterial gene OLAH and of at least one other target bacterial gene chosen from SLC1A2, IL1R2, FAM20A, RETN and MMP8.

According to another particular embodiment, step (a) comprises or consists in measuring the expression of a combination of two target genes chosen from the combinations in Table 6, and preferably those with a performance in terms of area under the ROC curve of the model of at least 0.86, and most particularly, of at least 0.88.

According to a preferred variant of this particular embodiment, step (a) comprises or consists in measuring the expression of a combination of a target viral gene and a target bacterial gene, said combination being chosen from the following combinations: SIGLEC1_IL1R2, IFI44L_OLAH and IFI27_1L1R2.

According to another embodiment, step (a) comprises or consists in measuring the expression of a combination of two target genes chosen from the following combinations: SIGLEC1_IL1R2; SIGLEC1_FAM20A; HERC6_IL1R2; SIGLEC1_OLAH; HERC6_OLAH; ISG15_IL1R2; SIGLEC1_RETN; HERC6_FAM20A; SLC1A2_OLAH; HERC6_RETN; SIGLEC1_SLC1A2; ISG15_OLAH; SIGLEC1_MMP8; HERC6_MMP8; IL1R2_OLAH; IL1R2_FAM20A; SIGLEC1_HERC6; HERC6_SLC1A2; SIGLEC1_ISG15; FAM20A_OLAH; ISG15_FAM20A; SLC1A2_IL1R2; ISG15_RETN; ISG15_MMP8 and ISG15_SLC1A2, and preferably chosen from the following gene combinations: SIGLEC1_IL1R2; SIGLEC1_FAM20A; HERC6_IL1R2; SIGLEC1_OLAH and HERC6_OLAH.

According to another particular embodiment, step (a) comprises or consists in measuring at least one target viral gene chosen from IFI27, SIGLEC1, ISG15, HERC6, RSAD2, OAS1, IFIT1 and IFI44L, the target bacterial gene OLAH, and at least one other target bacterial gene chosen from FAM20A, IL1R2, MMP8, RETN and SLC1A2, preferably from FAM20A, IL1R2 and MMP8.

According to another particular embodiment, step (a) comprises or consists in measuring at least one target viral gene chosen from IFI27, SIGLEC1, ISG15, HERC6, RSAD2, OAS1, IFIT1 and IFI44L, the target bacterial gene FAM20A, and at least one other target bacterial gene chosen from OLAH, IL1R2, MMP8, RETN and SLC1A2, preferably from OLAH, IL1R2 and MMP8.

According to another particular embodiment, step (a) comprises or consists in measuring the expression of the target viral genes SIGLEC1 and ISG15, and of one or more target bacterial genes chosen from OLAH, FAM20A, IL1R2, MMP8, RETN and SLC1A2.

According to a particular embodiment, step (a) comprises or consists in measuring the expression of the target viral genes SIGLEC1 and HERC6, and of one or more target bacterial genes chosen from OLAH, FAM20A, IL1R2, MMP8, RETN and SLC1A2.

According to a particular embodiment, step (a) comprises or consists in measuring the expression of the target viral genes SIGLEC1 and RSAD2, and of one or more target bacterial genes chosen from OLAH, FAM20A, IL1R2, MMP8, RETN and SLC1A2.

According to a preferred embodiment, step (a) comprises or consists in measuring the expression of the target viral genes SIGLEC1 and IFI27, and of one or more target bacterial genes chosen from OLAH, FAM20A, IL1R2, MMP8, RETN and SLC1A2.

According to a variant of this preferred embodiment, step (a) comprises or consists in measuring the expression of the target viral genes SIGLEC1 and IFI27, and of two to four target bacterial genes chosen from OLAH, FAM20A, IL1R2 and MMP8.

According to another particular embodiment, step (a) comprises or consists in measuring the expression of the target viral genes SIGLEC1 and OAS1, and of one or more target bacterial genes chosen from OLAH, FAM20A, IL1R2, MMP8, RETN and SLC1A2.

According to another particular embodiment, step (a) comprises or consists in measuring the expression of the target viral genes SIGLEC1 and IFIT1, and of one or more target bacterial genes chosen from OLAH, FAM20A, IL1R2, MMP8, RETN and SLC1A2, preferably chosen from OLAH and FAM20A.

According to another particular embodiment, step (a) comprises or consists in measuring the expression of the target viral genes SIGLEC1 and IFI44L, and of one or more target bacterial genes chosen from OLAH, FAM20A, IL1R2, MMP8, RETN and SLC1A2.

According to another particular embodiment, step (a) comprises or consists in measuring the expression of the target viral genes ISG15 and HERC6, and of one or more target bacterial genes chosen from OLAH, FAM20A, IL1R2, MMP8, RETN and SLC1A2.

According to another particular embodiment, step (a) comprises or consists in measuring the expression of the target viral genes ISG15 and RSAD2, and of one or more target bacterial genes chosen from OLAH, FAM20A, IL1R2, MMP8, RETN and SLC1A2.

According to another particular embodiment, step (a) comprises or consists in measuring the expression of the target viral genes ISG15 and IFI27, and of one or more target bacterial genes chosen from OLAH, FAM20A, IL1R2, MMP8, RETN and SLC1A2, preferably chosen from OLAH and FAM20A.

According to another particular embodiment, step (a) comprises or consists in measuring the expression of the target viral genes ISG15 and OAS1, and of one or more target bacterial genes chosen from OLAH, FAM20A, IL1R2, MMP8, RETN and SLC1A2.

According to another particular embodiment, step (a) comprises or consists in measuring the expression of the target viral genes ISG15 and IFIT1, and of one or more target bacterial genes chosen from OLAH, FAM20A, IL1R2, MMP8, RETN and SLC1A2.

According to another particular embodiment, step (a) comprises or consists in measuring the expression of the target viral genes ISG15 and IFI44L, and of one or more target bacterial genes chosen from OLAH, FAM20A, IL1R2, MMP8, RETN and SLC1A2.

According to another particular embodiment, step (a) comprises or consists in measuring the expression of the target viral genes HERC6 and RSAD2, and of one or more target bacterial genes chosen from OLAH, FAM20A, IL1R2, MMP8, RETN and SLC1A2.

According to another particular embodiment, step (a) comprises or consists in measuring the expression of the target viral genes HERC6 and IFI27, and of one or more target bacterial genes chosen from OLAH, FAM20A, IL1R2, MMP8, RETN and SLC1A2, preferably chosen from OLAH and FAM20A.

According to another particular embodiment, step (a) comprises or consists in measuring the expression of the target viral genes HERC6 and OAS1, and of one or more target bacterial genes chosen from OLAH, FAM20A, IL1R2, MMP8, RETN and SLC1A2.

According to another particular embodiment, step (a) comprises or consists in measuring the expression of the target viral genes HERC6 and IFIT1, and of one or more target bacterial genes chosen from OLAH, FAM20A, IL1R2, MMP8, RETN and SLC1A2.

According to another particular embodiment, step (a) comprises or consists in measuring the expression of the target viral genes HERC6 and IFI44L, and of one or more target bacterial genes chosen from OLAH, FAM20A, IL1R2, MMP8, RETN and SLC1A2.

According to another particular embodiment, step (a) comprises or consists in measuring the expression of the target viral genes RSAD2 and IFI27, and of one or more target bacterial genes chosen from OLAH, FAM20A, IL1R2, MMP8, RETN and SLC1A2.

According to another particular embodiment, step (a) comprises or consists in measuring the expression of the target viral genes RSAD2 and OAS1, and of one or more target bacterial genes chosen from OLAH, FAM20A, IL1R2, MMP8, RETN and SLC1A2.

According to a particular embodiment, step (a) comprises or consists in measuring the expression of the target viral genes RSAD2 and IFIT1, and of one or more target bacterial genes chosen from OLAH, FAM20A, IL1R2, MMP8, RETN and SLC1A2.

According to another particular embodiment, step (a) comprises or consists in measuring the expression of the target viral genes RSAD2 and IFI44L, and of one or more target bacterial genes chosen from OLAH, FAM20A, IL1R2, MMP8, RETN and SLC1A2.

According to another particular embodiment, step (a) comprises or consists in measuring the expression of the target viral genes IFI27 and OAS1, and of one or more target bacterial genes chosen from OLAH, FAM20A, IL1R2, MMP8, RETN and SLC1A2.

According to another particular embodiment, step (a) comprises or consists in measuring the expression of the target viral genes IFI27 and IFIT1, and of one or more target bacterial genes chosen from OLAH, FAM20A, IL1R2, MMP8, RETN and SLC1A2.

According to another particular embodiment, step (a) comprises or consists in measuring the expression of the target viral genes IFI27 and IFI44L, and of one or more target bacterial genes chosen from OLAH, FAM20A, IL1R2, MMP8, RETN and SLC1A2.

According to another particular embodiment, step (a) comprises or consists in measuring the expression of the target viral genes OAS1 and IFIT1, and of one or more target bacterial genes chosen from OLAH, FAM20A, IL1R2, MMP8, RETN and SLC1A2.

According to another particular embodiment, step (a) comprises or consists in measuring the expression of the target viral genes OAS1 and IFI44L, and of one or more target bacterial genes chosen from OLAH, FAM20A, IL1R2, MMP8, RETN and SLC1A2.

According to another particular embodiment, step (a) comprises or consists in measuring the expression of the target viral genes IFI44L and IFIT1, and of one or more target bacterial genes chosen from OLAH, FAM20A, IL1R2, MMP8, RETN and SLC1A2.

According to another particular embodiment, the measurement step comprises or consists in measuring at least one target viral gene chosen from SIGLEC1, ISG15 and HERC6, and at least one target bacterial gene chosen from OLAH, FAM20A, IL1R2, MMP8, RETN and SLC1A2. Preferably, according to this embodiment, the measurement step comprises or consists in measuring at least one target viral gene chosen from SIGLEC1, ISG15 and HERC6, and at least one target gene chosen from IL1R2, OLAH, and FAM20A.

According to another particular embodiment, the measurement step comprises or consists in measuring at least one target viral gene chosen from RSAD2, IFI27, OAS1, IFIT1 and IFI44L, and at least one target bacterial gene chosen from OLAH, FAM20A, IL1R2, MMP8, RETN and SLC1A2. Preferably, according to this embodiment, the measurement step comprises or consists in measuring at least one target viral gene chosen from RSAD2, IFI27, OAS1, IFIT1 and IFI44L, and at least one target bacterial gene chosen from IL1R2, OLAH and FAM20A.

According to another preferred embodiment, the measurement step comprises or consists in measuring at least one target viral gene chosen from SIGLEC1, ISG15 and HERC6, and at least two target bacterial genes chosen from SLC1A2, IL1R2, OLAH, FAM20A and RETN and MMP8, and preferably IL1R2, OLAH and FAM20A.

According to a variant of this particular embodiment, step (a) comprises or consists in measuring the expression of the target viral gene SIGLEC1, and of at least two target bacterial genes chosen from the group consisting of OLAH, SLC1A2, IL1R2, FAM20A, RETN and MMP8.

According to another variant of this particular embodiment, step (a) comprises or consists in measuring the expression of the target viral gene HERC6, and of at least two target bacterial genes chosen from the group consisting of OLAH, SLC1A2, IL1R2, FAM20A, RETN and MMP8.

According to another variant of this particular embodiment, step (a) comprises or consists in measuring the expression of the target viral gene ISG15, and of at least two target bacterial genes chosen from the group consisting of OLAH, SLC1A2, IL1R2, FAM20A, RETN and MMP8.

According to another variant of this particular embodiment, step (a) comprises or consists in measuring the expression of the target viral gene SIGLEC1, and of the target bacterial gene OLAH, and of at least one other target gene chosen from the group consisting of ISG15, HERC6, SLC1A2, IL1R2, FAM20A, RETN and MMP8.

According to another variant of this particular embodiment, step (a) comprises or consists in measuring the expression of the target viral gene SIGLEC1, and of the target bacterial gene IL1R2, and of at least one other target gene chosen from ISG15, HERC6, SLC1A2, OLAH, FAM20A, RETN and MMP8, and preferably chosen from SLC1A2, OLAH, FAM20A, RETN and MMP8.

According to another particular embodiment, step (a) comprises or consists in measuring the expression of the target viral genes SIGLEC1, ISG15 and HERC6, and of one or more target bacterial genes chosen from OLAH, FAM20A, IL1R2, MMP8, RETN and SLC1A2.

According to another particular embodiment, step (a) comprises or consists in measuring the expression of the target viral genes SIGLEC1, ISG15 and RSAD2, and of one or more bacterial genes chosen from OLAH, FAM20A, IL1R2, MMP8, RETN and SLC1A2.

According to another particular embodiment, step (a) comprises or consists in measuring the expression of the target viral genes SIGLEC1, ISG15 and IFI27, and of one or more target bacterial genes chosen from OLAH, FAM20A, IL1R2, MMP8, RETN and SLC1A2.

According to another particular embodiment, step (a) comprises or consists in measuring the expression of the target viral genes SIGLEC1, ISG15 and OAS1, and of one or more target bacterial genes chosen from OLAH, FAM20A, IL1R2, MMP8, RETN and SLC1A2.

According to another particular embodiment, step (a) comprises or consists in measuring the expression of the target viral genes SIGLEC1, ISG15 and IFIT1, and of one or more bacterial genes chosen from OLAH, FAM20A, IL1R2, MMP8, RETN and SLC1A2.

According to another particular embodiment, step (a) comprises or consists in measuring the expression of the target viral genes SIGLEC1, ISG15 and IFI44L, and of one or more target bacterial genes chosen from OLAH, FAM20A, IL1R2, MMP8, RETN and SLC1A2.

According to another particular embodiment, step (a) comprises or consists in measuring the expression of the target viral genes SIGLEC1, HERC6 and RSAD2, and of one or more bacterial genes chosen from OLAH, FAM20A, IL1R2, MMP8, RETN and SLC1A2.

According to another particular embodiment, step (a) comprises or consists in measuring the expression of the target viral genes SIGLEC1, HERC6 and IFI27, and of one or more target bacterial genes chosen from OLAH, FAM20A, IL1R2, MMP8, RETN and SLC1A2.

According to another particular embodiment, step (a) comprises or consists in measuring the expression of the target viral genes SIGLEC1, HERC6 and OAS1, and of one or more target bacterial genes chosen from OLAH, FAM20A, IL1R2, MMP8, RETN and SLC1A2.

According to another particular embodiment, step (a) comprises or consists in measuring the expression of the target viral genes SIGLEC1, HERC6 and IFIT1, and of one or more bacterial genes chosen from OLAH, FAM20A, IL1R2, MMP8, RETN and SLC1A2.

According to another particular embodiment, step (a) comprises or consists in measuring the expression of the target viral genes SIGLEC1, HERC6 and IFI44L, and of one or more target bacterial genes chosen from OLAH, FAM20A, IL1R2, MMP8, RETN and SLC1A2.

According to another particular embodiment, step (a) comprises or consists in measuring the expression of the target viral genes SIGLEC1, RSAD2 and IFI27, and of one or more target bacterial genes chosen from OLAH, FAM20A, IL1R2, MMP8, RETN and SLC1A2.

According to another particular embodiment, step (a) comprises or consists in measuring the expression of the target viral genes SIGLEC1, RSAD2 and OAS1, and of one or more target bacterial genes chosen from OLAH, FAM20A, IL1R2, MMP8, RETN and SLC1A2.

According to another particular embodiment, step (a) comprises or consists in measuring the expression of the target viral genes SIGLEC1, RSAD2 and IFIT1, and of one or more bacterial genes chosen from OLAH, FAM20A, IL1R2, MMP8, RETN and SLC1A2.

According to another particular embodiment, step (a) comprises or consists in measuring the expression of the target viral genes SIGLEC1, RSAD2 and IFI44L, and of one or more target bacterial genes chosen from OLAH, FAM20A, IL1R2, MMP8, RETN and SLC1A2.

According to another particular embodiment, step (a) comprises or consists in measuring the expression of the target viral genes SIGLEC1, IFI27 and OAS1, and of one or more target bacterial genes chosen from OLAH, FAM20A, IL1R2, MMP8, RETN and SLC1A2.

According to another particular embodiment, step (a) comprises or consists in measuring the expression of the target viral genes SIGLEC1, IFI27 and IFIT1, and of one or more bacterial genes chosen from OLAH, FAM20A, IL1R2, MMP8, RETN and SLC1A2.

According to another particular embodiment, step (a) comprises or consists in measuring the expression of the target viral genes SIGLEC1, IFI27 and IFI44L, and of one or more target bacterial genes chosen from OLAH, FAM20A, IL1R2, MMP8, RETN and SLC1A2.

According to another particular embodiment, step (a) comprises or consists in measuring the expression of the target viral genes SIGLEC1, IFIT1 and OAS1, and of one or more target bacterial genes chosen from OLAH, FAM20A, IL1R2, MMP8, RETN and SLC1A2.

According to another particular embodiment, step (a) comprises or consists in measuring the expression of the target viral genes SIGLEC1, OAS1 and OAS1, and of one or more target bacterial genes chosen from OLAH, FAM20A, IL1R2, MMP8, RETN and SLC1A2.

According to another particular embodiment, step (a) comprises or consists in measuring the expression of the target viral genes SIGLEC1, IFI27 and IFI44L, and of one or more target bacterial genes chosen from OLAH, FAM20A, IL1R2, MMP8, RETN and SLC1A2.

According to another particular embodiment, step (a) comprises or consists in measuring the expression of the target viral genes ISG15, HERC6 and RSAD2, and of one or more bacterial genes chosen from OLAH, FAM20A, IL1R2, MMP8, RETN and SLC1A2.

According to another particular embodiment, step (a) comprises or consists in measuring the expression of the target viral genes ISG15, HERC6 and IFI27, and of one or more target bacterial genes chosen from OLAH, FAM20A, IL1R2, MMP8, RETN and SLC1A2.

According to another particular embodiment, step (a) comprises or consists in measuring the expression of the target viral genes ISG15, HERC6 and OAS1, and of one or more target bacterial genes chosen from OLAH, FAM20A, IL1R2, MMP8, RETN and SLC1A2.

According to another particular embodiment, step (a) comprises or consists in measuring the expression of the target viral genes ISG15, HERC6 and IFIT1, and of one or more bacterial genes chosen from OLAH, FAM20A, IL1R2, MMP8, RETN and SLC1A2.

According to another particular embodiment, step (a) comprises or consists in measuring the expression of the target viral genes ISG15, HERC6 and IFI44L, and of one or more target bacterial genes chosen from OLAH, FAM20A, IL1R2, MMP8, RETN and SLC1A2.

According to another particular embodiment, step (a) comprises or consists in measuring the expression of the target viral genes ISG15, RSAD2 and IFI27, and of one or more target bacterial genes chosen from OLAH, FAM20A, IL1R2, MMP8, RETN and SLC1A2.

According to another particular embodiment, step (a) comprises or consists in measuring the expression of the target viral genes ISG15, RSAD2 and OAS1, and of one or more target bacterial genes chosen from OLAH, FAM20A, IL1R2, MMP8, RETN and SLC1A2.

According to another particular embodiment, step (a) comprises or consists in measuring the expression of the target viral genes ISG15, RSAD2 and IFIT1, and of one or more bacterial genes chosen from OLAH, FAM20A, IL1R2, MMP8, RETN and SLC1A2.

According to another particular embodiment, step (a) comprises or consists in measuring the expression of the target viral genes ISG15, RSAD2 and IFI44L, and of one or more target bacterial genes chosen from OLAH, FAM20A, IL1R2, MMP8, RETN and SLC1A2.

According to another particular embodiment, step (a) comprises or consists in measuring the expression of the target viral genes ISG15, IFI27 and OAS1, and of one or more target bacterial genes chosen from OLAH, FAM20A, IL1R2, MMP8, RETN and SLC1A2.

According to another particular embodiment, step (a) comprises or consists in measuring the expression of the target viral genes ISG15, IFI27 and IFIT1, and of one or more bacterial genes chosen from OLAH, FAM20A, IL1R2, MMP8, RETN and SLC1A2.

According to another particular embodiment, step (a) comprises or consists in measuring the expression of the target viral genes ISG15, IFI27 and IFI44L, and of one or more target bacterial genes chosen from OLAH, FAM20A, IL1R2, MMP8, RETN and SLC1A2.

According to another particular embodiment, step (a) comprises or consists in measuring the expression of the target viral genes ISG15, OAS1 and IFIT1, and of one or more bacterial genes chosen from OLAH, FAM20A, IL1R2, MMP8, RETN and SLC1A2.

According to another particular embodiment, step (a) comprises or consists in measuring the expression of the target viral genes ISG15, OAS1 and IFI44L, and of one or more target bacterial genes chosen from OLAH, FAM20A, IL1R2, MMP8, RETN and SLC1A2.

According to another particular embodiment, step (a) comprises or consists in measuring the expression of the target viral genes ISG15, IFIT1 and IFI44L, and of one or more target bacterial genes chosen from OLAH, FAM20A, IL1R2, MMP8, RETN and SLC1A2.

According to another particular embodiment, step (a) comprises or consists in measuring the expression of the target viral genes HERC6, RSAD2 and IFI27, and of one or more target bacterial genes chosen from OLAH, FAM20A, IL1R2, MMP8, RETN and SLC1A2.

According to another particular embodiment, step (a) comprises or consists in measuring the expression of the target viral genes HERC6, RSAD2 and OAS1, and of one or more target bacterial genes chosen from OLAH, FAM20A, IL1R2, MMP8, RETN and SLC1A2.

According to another particular embodiment, step (a) comprises or consists in measuring the expression of the target viral genes HERC6, RSAD2 and IFIT1, and of one or more bacterial genes chosen from OLAH, FAM20A, IL1R2, MMP8, RETN and SLC1A2.

According to another particular embodiment, step (a) comprises or consists in measuring the expression of the target viral genes HERC6, RSAD2 and IFI44L, and of one or more target bacterial genes chosen from OLAH, FAM20A, IL1R2, MMP8, RETN and SLC1A2.

According to another particular embodiment, step (a) comprises or consists in measuring the expression of the target viral genes HERC6, IFI27 and OAS1, and of one or more target bacterial genes chosen from OLAH, FAM20A, IL1R2, MMP8, RETN and SLC1A2.

According to another particular embodiment, step (a) comprises or consists in measuring the expression of the target viral genes HERC6, IFI27 and IFIT1, and of one or more bacterial genes chosen from OLAH, FAM20A, IL1R2, MMP8, RETN and SLC1A2.

According to another particular embodiment, step (a) comprises or consists in measuring the expression of the target viral genes HERC6, IFI27 and IFI44L, and of one or more target bacterial genes chosen from OLAH, FAM20A, IL1R2, MMP8, RETN and SLC1A2.

According to another particular embodiment, step (a) comprises or consists in measuring the expression of the target viral genes HERC6, OAS1 and IFIT1, and of one or more bacterial genes chosen from OLAH, FAM20A, IL1R2, MMP8, RETN and SLC1A2.

According to another particular embodiment, step (a) comprises or consists in measuring the expression of the target viral genes HERC6, OAS1 and IFI44L, and of one or more target bacterial genes chosen from OLAH, FAM20A, IL1R2, MMP8, RETN and SLC1A2.

According to another particular embodiment, step (a) comprises or consists in measuring the expression of the target viral genes HERC6, IFI44L and IFIT1, and of one or more bacterial genes chosen from OLAH, FAM20A, IL1R2, MMP8, RETN and SLC1A2.

According to another particular embodiment, step (a) comprises or consists in measuring the expression of the target viral genes RSAD2, OAS1 and IFI27, and of one or more target bacterial genes chosen from OLAH, FAM20A, IL1R2, MMP8, RETN and SLC1A2.

According to another particular embodiment, step (a) comprises or consists in measuring the expression of the target viral genes RSAD2, IFI27 and IFIT1, and of one or more bacterial genes chosen from OLAH, FAM20A, IL1R2, MMP8, RETN and SLC1A2.

According to another particular embodiment, step (a) comprises or consists in measuring the expression of the target viral genes RSAD2, IFI27 and IFI44L, and of one or more target bacterial genes chosen from OLAH, FAM20A, IL1R2, MMP8, RETN and SLC1A2.

According to another particular embodiment, step (a) comprises or consists in measuring the expression of the target viral genes RSAD2, OAS1 and IFIT1, and of one or more target bacterial genes chosen from OLAH, FAM20A, IL1R2, MMP8, RETN and SLC1A2.

According to another particular embodiment, step (a) comprises or consists in measuring the expression of the target viral genes RSAD2, OAS1 and IFI44L, and of one or more target bacterial genes chosen from OLAH, FAM20A, IL1R2, MMP8, RETN and SLC1A2.

According to another particular embodiment, step (a) comprises or consists in measuring the expression of the target viral genes RSAD2, IFIT1 and IFI44L, and of one or more target bacterial genes chosen from OLAH, FAM20A, IL1R2, MMP8, RETN and SLC1A2.

According to another particular embodiment, step (a) comprises or consists in measuring the expression of the target viral genes IFI27, OAS1 and IFIT1, and of one or more target bacterial genes chosen from OLAH, FAM20A, IL1R2, MMP8, RETN and SLC1A2.

According to another particular embodiment, step (a) comprises or consists in measuring the expression of the target viral genes IFI27, OAS1 and IFI44L, and of one or more target bacterial genes chosen from OLAH, FAM20A, IL1R2, MMP8, RETN and SLC1A2.

According to another particular embodiment, step (a) comprises or consists in measuring the expression of the target viral genes IFI27, IFIT1 and IFI44L, and of one or more target bacterial genes chosen from OLAH, FAM20A, IL1R2, MMP8, RETN and SLC1A2.

According to another particular embodiment, step (a) comprises or consists in measuring the expression of the target viral genes OAS1, IFIT1 and IFI44L, and of one or more target bacterial genes chosen from OLAH, FAM20A, IL1R2, MMP8, RETN and SLC1A2.

According to another particular embodiment, step (a) comprises or consists in measuring the expression of a combination of three target genes chosen from those listed in Table 7, and preferably those with a performance in terms of area under the ROC curve of the model of at least 0.86, at least 0.88, and most particularly at least 0.90.

According to another variant of this particular embodiment, the measurement step comprises or consists in measuring the expression of a combination of three target genes chosen from the following combinations: SIGLEC1_IL1R2_FAM20A, HERC6_IL1R2_FAM20A, SIGLEC1_SLC1A2_OLAH, SIGLEC1_HERC6_IL1R2, SIGLEC1_FAM20A_OLAH, SIGLEC1_IL1R2_MMP8, SIGLEC1_ISG15_IL1R2, SIGLEC1_SLC1A2_IL1R2, SIGLEC1 IL1R2_OLAH, SIGLEC1_SLC1A2_FAM20A, HERC6_FAM20A_OLAH, SIGLEC1_FAM20A_RETN, SIGLEC1_HERC6_FAM20A, SIGLEC1_FAM20A_MMP8, SIGLEC1_IL1R2_RETN, ISG15_IL1R2_FAM20A, SIGLEC1_ISG15_FAM20A, ISG15_HERC6_IL1R2, HERC6_IL1R2_OLAH, SIGLEC1_HERC6_OLAH, HERC6_SLC1A2_OLAH, SIGLEC1_ISG15_OLAH, HERC6_IL1R2_RETN, HERC6_IL1R2_MMP8, SIGLEC1_OLAH_MMP8, SIGLEC1_OLAH_RETN, HERC6_SLC1A2_IL1R2, ISG15_SLC1A2_OLAH, HERC6_FAM20A_RETN, HERC6_OLAH_RETN, ISG15_SLC1A2_IL1R2, HERC6_OLAH_MMP8, SIGLEC1_HERC6_RETN, SIGLEC1_SLC1A2_RETN, ISG15_IL1R2_OLAH, ISG15_HERC6_OLAH, ISG15_IL1R2_RETN, SIGLEC1_SLC1A2_MMP8, ISG15_HERC6_RETN, ISG15_FAM20A_OLAH, ISG15_IL1R2_MMP8, SIGLEC1_ISG15_RETN, HERC6_SLC1A2_FAM20A, ISG15_HERC6_FAM20A, HERC6_FAM20A_MMP8, SLC1A2_FAM20A_OLAH, SIGLEC1_RETN_MMP8, HERC6_SLC1A2_RETN, SIGLEC1_HERC6_SLC1A2, SLC1A2_IL1R2_OLAH, SIGLEC1_HERC6_MMP8, ISG15_OLAH_RETN, SIGLEC1_ISG15_SLC1A2, ISG15_OLAH_MMP8, SLC1A2_OLAH_MMP8, SLC1A2_OLAH_RETN, HERC6 RETN_MMP8, HERC6_SLC1A2_MMP8, SIGLEC1_ISG15_MMP8, IL1R2_FAM20A_OLAH, ISG15_HERC6_MMP8, ISG15_SLC1A2_FAM20A, ISG15_FAM20A_RETN, IL1R2_OLAH_MMP8, SLC1A2_IL1R2_FAM20A, IL1R2_OLAH_RETN, SIGLEC1_ISG15_HERC6, FAM20A_OLAH_MMP8, ISG15_SLC1A2_RETN, IL1R2_FAM20A_MMP8, ISG15_HERC6_SLC1A2, ISG15_SLC1A2_MMP8, ISG15_FAM20A_MMP8, IL1R2_FAM20A_RETN, SLC1A2_IL1R2_RETN, FAM20A_OLAH_RETN, OLAH_RETN_MMP8, SLC1A2_IL1R2_MMP8, ISG15_RETN_MMP8, IL1R2 RETN_MMP8, SLC1A2_FAM20A_RETN, SLC1A2_FAM20A_MMP8, SLC1A2_RETN_MMP8 and FAM20A_RETN_MMP8. Preferably, according to this variant, the measurement step comprises or consists in measuring the expression of a combination of three target genes chosen from the following combinations: SIGLEC1_IL1R2_FAM20A, HERC6_IL1R2_FAM20A, SIGLEC1_SLC1A2_OLAH, SIGLEC1_HERC6_IL1R2, SIGLEC1_FAM20A_OLAH, SIGLEC1_IL1R2_MMP8, SIGLEC1_ISG15_IL1R2, SIGLEC1_SLC1A2_IL1R2, SIGLEC1_IL1R2_OLAH, SIGLEC1_SLC1A2_FAM20A and HERC6_FAM20A_OLAH, and more preferably from SIGLEC1_IL1R2_FAM20A, HERC6_IL1R2_FAM20A and SIGLEC1_SLC1A2_OLAH.

According to another variant of this particular embodiment, step (a) comprises or consists in measuring the expression of a combination of three target genes chosen from the following combinations: IFI27_OLAH_FAM20A, IFI27_IL1R2_FAM20A, IFI27_OLAH_MMP8, IFI27_HERC6_OLAH, IFI27_IL1R2_OLAH, IFI27_ISG15_OLAH, IFI27_OLAH_RETN, IFI27_SLC1A2_OLAH, IFI27_SIGLEC1_OLAH, IFI27_SIGLEC1_IL1R2, IFI27_IFI44L_OLAH, IFI27_IFIT1_OLAH, IFI27_OAS1_OLAH, IFI27_HERC6_IL1R2, RSAD2_IFI27_OLAH, IFI27_IL1R2_RETN, IFI27_IFI44L_IL1R2, IFI27_IFIT1_IL1R2, IFI27_ISG15_IL1R2 and RSAD2_IFI27_IL1R2.

According to another particular embodiment, step (a) comprises or consists in measuring the expression of at least four target genes.

According to a variant of this embodiment, the measurement step comprises or consists in measuring at least two target genes chosen from SIGLEC1, ISG15 and HERC6, and at least two target genes chosen OLAH, FAM20A, IL1R2, MMP8, RETN and SLC1A2, and preferably IL1R2, OLAH and FAM20A.

According to another variant of this embodiment, the measurement step comprises or consists in measuring SIGLEC1 and at least three target genes chosen from SLC1A2, IL1R2, OLAH, FAM20A and RETN.

According to another variant of this embodiment, the measurement step comprises or consists in measuring the expression of the target genes SIGLEC1 and IL1R2, and of at least two other target genes chosen from ISG15, HERC6, SLC1A2, OLAH, FAM20A, RETN and MMP8, preferably from SLC1A2, OLAH and, FAM20A.

According to another variant of this embodiment, the measurement step comprises or consists in measuring the expression of the target genes SIGLEC1 and HERC6, and of at least two other target genes chosen from ISG15, IL1R2, SLC1A2, OLAH, FAM20A, RETN and MMP8. According to another variant of this particular embodiment, the measurement step comprises or consists in measuring the expression of the target genes SIGLEC1, IL1R2 and FAM20A, and of at least one other target gene chosen from ISG15, HERC6, SLC1A2, OLAH, RETN and MMP8.

According to another variant of this embodiment, the measurement step comprises or consists in measuring the expression of the target genes SIGLEC1 and SLC1A2, and of at least two other target genes chosen from ISG15, IL1R2, HERC6, OLAH, FAM20A, RETN and MMP8, and preferably IL1R2, OLAH, FAM20A and MMP8.

According to another preferred embodiment, step (a) comprises or consists in measuring the expression of the target viral gene IFI27, of another target viral gene chosen from HERC6, SIGLEC1, IFI44L and ISG15, of the target bacterial gene FAM20A and of another target bacterial gene chosen from OLAH and IL1R2.

According to another variant of this particular embodiment, step (a) comprises or consists in measuring the expression of a combination of four target genes chosen from those listed in Table 8, and preferably those in this table which have a performance in terms of area under the ROC curve of the model of at least 0.86, at least 0.88, and most particularly, at least 0.90.

According to another variant of this particular embodiment, the measurement step comprises or consists in measuring the expression of a combination of four target genes chosen from the combinations presented in Table 3 below:

TABLE 3
Preferred combinations of four genes

| | |
|---|---|
| SIGLEC1_IL1R2_FAM20A_MMP8 | ISG15_HERC6_SLC1A2_IL1R2 |
| SIGLEC1_HERC6_IL1R2_FAM20A | HERC6_SLC1A2_OLAH_RETN |
| SIGLEC1_ISG15_IL1R2_FAM20A | ISG15_SLC1A2_IL1R2_OLAH |
| SIGLEC1_IL1R2_FAM20A_OLAH | HERC6_IL1R2_OLAH_RETN |
| SIGLEC1_IL1R2_FAM20A_RETN | HERC6_IL1R2_OLAH_MMP8 |
| SIGLEC1_SLC1A2_IL1R2_FAM20A | HERC6_IL1R2_RETN_MMP8 |
| SIGLEC1_SLC1A2_FAM20A_OLAH | SIGLEC1_ISG15_OLAH_MMP8 |
| ISG15_HERC6_IL1R2_FAM20A | SIGLEC1_ISG15_OLAH_RETN |
| SIGLEC1_FAM20A_OLAH_MMP8 | ISG15_HERC6_OLAH_RETN |
| HERC6_IL1R2_FAM20A_OLAH | HERC6_SLC1A2_IL1R2_RETN |
| SIGLEC1_SLC1A2_OLAH_MMP8 | HERC6_SLC1A2_FAM20A_RETN |
| SIGLEC1_ISG15_SLC1A2_OLAH | HERC6_OLAH_RETN_MMP8 |
| SIGLEC1_HERC6_FAM20A_OLAH | HERC6_SLC1A2_IL1R2_MMP8 |
| SIGLEC1_SLC1A2_IL1R2_OLAH | SIGLEC1_HERC6_SLC1A2_RETN |
| SIGLEC1_ISG15_FAM20A_OLAH | ISG15_SLC1A2_OLAH_MMP8 |
| HERC6_IL1R2_FAM20A_MMP8 | ISG15_SLC1A2_OLAH_RETN |
| SIGLEC1_HERC6_SLC1A2_OLAH | SIGLEC1_ISG15_HERC6_RETN |
| SIGLEC1_HERC6_IL1R2_MMP8 | ISG15_SLC1A2_IL1R2_RETN |
| HERC6_IL1R2_FAM20A_RETN | ISG15_HERC6_OLAH_MMP8 |
| SIGLEC1_FAM20A_RETN_MMP8 | HERC6_FAM20A_RETN_MMP8 |
| SIGLEC1_HERC6_FAM20A_RETN | SIGLEC1_ISG15_SLC1A2_RETN |
| SIGLEC1_SLC1A2_OLAH_RETN | SIGLEC1_HERC6_RETN_MMP8 |
| SIGLEC1_ISG15_HERC6_IL1R2 | ISG15_SLC1A2_IL1R2_MMP8 |
| SIGLEC1_FAM20A_OLAH_RETN | ISG15_IL1R2_OLAH_MMP8 |
| SIGLEC1_HERC6_IL1R2_OLAH | SIGLEC1_HERC6_SLC1A2_MMP8 |
| SIGLEC1_IL1R2_OLAH_MMP8 | HERC6_SLC1A2_FAM20A_MMP8 |
| HERC6_SLC1A2_IL1R2_FAM20A | ISG15_IL1R2_OLAH_RETN |
| SIGLEC1_SLC1A2_IL1R2_MMP8 | SIGLEC1_SLC1A2_RETN_MMP8 |
| SIGLEC1_ISG15_SLC1A2_IL1R2 | ISG15_HERC6_SLC1A2_FAM20A |
| SIGLEC1_HERC6_SLC1A2_IL1R2 | ISG15_HERC6_FAM20A_MMP8 |
| SIGLEC1_SLC1A2_IL1R2_RETN | ISG15_HERC6_SLC1A2_RETN |
| HERC6_SLC1A2_FAM20A_OLAH | ISG15_IL1R2_RETN_MMP8 |
| SIGLEC1_ISG15_FAM20A_RETN | SIGLEC1_ISG15_SLC1A2_MMP8 |
| SIGLEC1_ISG15_IL1R2_MMP8 | SLC1A2_IL1R2_FAM20A_OLAH |
| SIGLEC1_SLC1A2_FAM20A_MMP8 | ISG15_FAM20A_OLAH_MMP8 |
| SIGLEC1_ISG15_IL1R2_OLAH | SLC1A2_FAM20A_OLAH_MMP8 |
| SIGLEC1_ISG15_SLC1A2_FAM20A | ISG15_FAM20A_OLAH_RETN |
| SIGLEC1_HERC6_IL1R2_RETN | ISG15_HERC6_RETN_MMP8 |
| SIGLEC1_SLC1A2_FAM20A_RETN | SIGLEC1_ISG15_RETN_MMP8 |
| SIGLEC1_HERC6_SLC1A2_FAM20A | SLC1A2_FAM20A_OLAH_RETN |
| HERC6_FAM20A_OLAH_RETN | SIGLEC1_ISG15_HERC6_SLC1A2 |
| SIGLEC1_IL1R2_RETN_MMP8 | ISG15_OLAH_RETN_MMP8 |
| HERC6_FAM20A_OLAH_MMP8 | HERC6_SLC1A2_RETN_MMP8 |
| ISG15_HERC6_FAM20A_OLAH | ISG15_HERC6_SLC1A2_MMP8 |
| ISG15_SLC1A2_IL1R2_FAM20A | SLC1A2_IL1R2_OLAH_RETN |
| SIGLEC1_ISG15_HERC6_FAM20A | SIGLEC1_ISG15_HERC6_MMP8 |
| ISG15_IL1R2_FAM20A_OLAH | SLC1A2_IL1R2_OLAH_MMP8 |
| SIGLEC1_ISG15_FAM20A_MMP8 | ISG15_SLC1A2_FAM20A_RETN |
| SIGLEC1_ISG15_IL1R2_RETN | SLC1A2_OLAH_RETN_MMP8 |
| SIGLEC1_IL1R2_OLAH_RETN | IL1R2_FAM20A_OLAH_MMP8 |
| SIGLEC1_HERC6_FAM20A_MMP8 | ISG15_SLC1A2_FAM20A_MMP8 |
| ISG15_HERC6_FAM20A_RETN | IL1R2_FAM20A_OLAH_RETN |
| ISG15_IL1R2_FAM20A_MMP8 | ISG15_FAM20A_RETN_MMP8 |
| ISG15_HERC6_IL1R2_RETN | SLC1A2_IL1R2_FAM20A_MMP8 |
| ISG15_SLC1A2_FAM20A_OLAH | SLC1A2_IL1R2_FAM20A_RETN |
| SIGLEC1_HERC6_OLAH_MMP8 | ISG15_SLC1A2_RETN_MMP8 |
| ISG15_HERC6_IL1R2_MMP8 | IL1R2_OLAH_RETN_MMP8 |
| ISG15_HERC6_IL1R2_OLAH | FAM20A_OLAH_RETN_MMP8 |
| SIGLEC1_HERC6_OLAH_RETN | IL1R2_FAM20A_RETN_MMP8 |
| ISG15_HERC6_SLC1A2_OLAH | SLC1A2_IL1R2_RETN_MMP8 |
| ISG15_IL1R2_FAM20A_RETN | HERC6_SLC1A2_OLAH_MMP8 |
| SIGLEC1_OLAH_RETN_MMP8 | SIGLEC1_ISG15_HERC6_OLAH |
| HERC6_SLC1A2_IL1R2_OLAH | |

Preferably, according to this variant, the measurement step comprises or consists in measuring the expression of a combination of four target genes chosen from the following combinations: SIGLEC1_IL1R2_FAM20A_MMP8, SIGLEC1_HERC6_IL1R2_FAM20A, SIGLEC1_ISG15_IL1R2_FAM20A, SIGLEC1_IL1R2_FAM20A_OLAH, SIGLEC1_IL1R2_FAM20A_RETN, SIGLEC1_SLC1A2_IL1R2_FAM20A, SIGLEC1_SLC1A2_FAM20A_OLAH, ISG15_HERC6_IL1R2_FAM20A, SIGLEC1_FAM20A_OLAH_MMP8 and HERC6_IL1R2_FAM20A_OLAH.

According to another particular embodiment, step (a) comprises or consists in measuring the expression of at least five target genes.

According to a variant of this embodiment, the measurement step comprises or consists in measuring at least two target genes chosen from SIGLEC1, ISG15 and HERC6, and at least three other target genes chosen from IL1R2, SLC1A2, OLAH, FAM20A, RETN and MMP8.

According to another variant of this embodiment, the measurement step comprises or consists in measuring the expression of the target genes SIGLEC1, ISG15 and HERC6, and of at least two other target genes chosen from IL1R2, SLC1A2, OLAH, FAM20A, RETN and MMP8, preferably from IL1R2, OLAH and FAM20A.

According to a variant of this embodiment, the measurement step comprises or consists in measuring the expression of the target genes SIGLEC1 and ISG15 and of at least three other target genes chosen from HERC6, IL1R2, SLC1A2, OLAH, FAM20A, RETN and MMP8, and preferably from IL1R2, OLAH, FAM20A and MMP8.

According to a variant of this embodiment, the measurement step comprises or consists in measuring the expression of the target genes SIGLEC1 and IL1R2, and of at least three other target genes chosen from HERC6, ISG15, SLC1A2, OLAH, FAM20A, RETN and MMP8, preferably from OLAH, SLC1A2, FAM20A and MMP8.

According to a variant of this embodiment, the measurement step comprises or consists in measuring the expression of the target genes SIGLEC1 and MMP8 and of at least three other target genes chosen from HERC6, ISG15, SLC1A2, OLAH, FAM20A, RETN and IL1R2, preferably OLAH, SLC1A2, FAM20A and IL1R2.

According to a variant of this embodiment, the measurement step comprises or consists in measuring the expression of the target genes SIGLEC1 and HERC6 and of at least three other target genes chosen from ISG15, IL1R2, SLC1A2, OLAH, FAM20A, RETN and MMP8, and preferably from IL1R2, OLAH, FAM20A and MMP8.

According to another variant of this embodiment, the measurement step comprises or consists in measuring the expression of the target genes SIGLEC1, FAM20A and MMP8, and of at least two other target genes chosen from IL1R2, ISG15, HERC6, SLC1A2, OLAH and RETN, preferably from IL1R2, ISG15, SLC1A2 and OLAH.

According to a variant of this embodiment, the measurement step comprises or consists in measuring the expression of the target genes SIGLEC1, IL1R2 and HERC6, and of at least two other target genes chosen from ISG15, SLC1A2, OLAH, FAM20A, RETN and MMP8.

According to another variant of this embodiment, the measurement step comprises or consists in measuring the expression of the target genes SIGLEC1, IL1R2 and ISG15, and of at least two other target genes chosen from HERC6, SLC1A2, OLAH, FAM20A, RETN and MMP8.

According to another variant of this embodiment, the measurement step comprises or consists in measuring the expression of a combination of five target genes chosen from the combinations presented in Table 4 below:

TABLE 4
Preferred combinations of five genes

| | |
|---|---|
| SIGLEC1_HERC6_IL1R2_FAM20A_MMP8 | SIGLEC1_ISG15_SLC1A2_IL1R2_RETN |
| SIGLEC1_IL1R2_FAM20A_OLAH_MMP8 | SIGLEC1_ISG15_SLC1A2_FAM20A_RETN |
| SIGLEC1_ISG15_IL1R2_FAM20A_MMP8 | SIGLEC1_HERC6_SLC1A2_IL1R2_RETN |
| SIGLEC1_IL1R2_FAM20A_RETN_MMP8 | HERC6_SLC1A2_IL1R2_FAM20A_RETN |
| SIGLEC1_SLC1A2_IL1R2_FAM20A_MMP8 | SIGLEC1_HERC6_SLC1A2_OLAH_RETN |
| SIGLEC1_ISG15_HERC6_IL1R2_FAM20A | SIGLEC1_SLC1A2_IL1R2_RETN_MMP8 |
| SIGLEC1_HERC6_IL1R2_FAM20A_OLAH | SIGLEC1_ISG15_IL1R2_RETN_MMP8 |
| SIGLEC1_SLC1A2_FAM20A_OLAH_MMP8 | SIGLEC1_HERC6_SLC1A2_FAM20A_MMP8 |
| SIGLEC1_ISG15_IL1R2_FAM20A_OLAH | HERC6_SLC1A2_IL1R2_FAM20A_MMP8 |
| SIGLEC1_ISG15_IL1R2_FAM20A_RETN | HERC6_FAM20A_OLAH_RETN_MMP8 |
| SIGLEC1_HERC6_IL1R2_FAM20A_RETN | SIGLEC1_ISG15_HERC6_SLC1A2_FAM20A |
| SIGLEC1_HERC6_SLC1A2_IL1R2_FAM20A | HERC6_SLC1A2_FAM20A_OLAH_RETN |
| SIGLEC1_SLC1A2_IL1R2_FAM20A_OLAH | ISG15_HERC6_FAM20A_OLAH_RETN |
| SIGLEC1_IL1R2_FAM20A_OLAH_RETN | SIGLEC1_IL1R2_OLAH_RETN_MMP8 |
| SIGLEC1_SLC1A2_IL1R2_FAM20A_RETN | SIGLEC1_ISG15_HERC6_IL1R2_RETN |
| SIGLEC1_ISG15_SLC1A2_IL1R2_FAM20A | SIGLEC1_HERC6_IL1R2_OLAH_RETN |
| SIGLEC1_ISG15_SLC1A2_FAM20A_OLAH | ISG15_HERC6_FAM20A_OLAH_MMP8 |
| SIGLEC1_HERC6_SLC1A2_FAM20A_OLAH | SIGLEC1_ISG15_HERC6_FAM20A_MMP8 |
| SIGLEC1_HERC6_FAM20A_OLAH_MMP8 | SIGLEC1_ISG15_IL1R2_OLAH_RETN |
| SIGLEC1_FAM20A_OLAH_RETN_MMP8 | SIGLEC1_HERC6_OLAH_RETN_MMP8 |
| SIGLEC1_ISG15_FAM20A_OLAH_MMP8 | ISG15_IL1R2_FAM20A_OLAH_MMP8 |
| ISG15_HERC6_IL1R2_FAM20A_OLAH | ISG15_SLC1A2_IL1R2_FAM20A_RETN |
| SIGLEC1_SLC1A2_FAM20A_OLAH_RETN | ISG15_HERC6_IL1R2_RETN_MMP8 |
| ISG15_HERC6_IL1R2_FAM20A_MMP8 | ISG15_SLC1A2_IL1R2_FAM20A_MMP8 |
| ISG15_HERC6_IL1R2_FAM20A_RETN | SIGLEC1_ISG15_HERC6_OLAH_RETN |
| HERC6_IL1R2_FAM20A_OLAH_MMP8 | ISG15_HERC6_SLC1A2_IL1R2_OLAH |
| SIGLEC1_ISG15_HERC6_FAM20A_OLAH | ISG15_HERC6_SLC1A2_OLAH_MMP8 |
| SIGLEC1_HERC6_FAM20A_RETN_MMP8 | ISG15_HERC6_SLC1A2_FAM20A_RETN |
| SIGLEC1_ISG15_SLC1A2_OLAH_MMP8 | SIGLEC1_ISG15_HERC6_OLAH_MMP8 |
| SIGLEC1_SLC1A2_IL1R2_OLAH_MMP8 | ISG15_HERC6_SLC1A2_OLAH_RETN |
| SIGLEC1_ISG15_SLC1A2_IL1R2_OLAH | SIGLEC1_ISG15_OLAH_RETN_MMP8 |
| SIGLEC1_HERC6_FAM20A_OLAH_RETN | ISG15_IL1R2_FAM20A_OLAH_RETN |
| SIGLEC1_HERC6_SLC1A2_OLAH_MMP8 | ISG15_SLC1A2_FAM20A_OLAH_MMP8 |
| HERC6_IL1R2_FAM20A_OLAH_RETN | HERC6_SLC1A2_IL1R2_OLAH_RETN |
| SIGLEC1_ISG15_HERC6_FAM20A_RETN | ISG15_HERC6_IL1R2_OLAH_MMP8 |
| SIGLEC1_ISG15_FAM20A_RETN_MMP8 | ISG15_HERC6_IL1R2_OLAH_RETN |
| SIGLEC1_SLC1A2_IL1R2_OLAH_RETN | ISG15_IL1R2_FAM20A_RETN_MMP8 |
| SIGLEC1_HERC6_IL1R2_OLAH_MMP8 | HERC6_IL1R2_OLAH_RETN_MMP8 |
| SIGLEC1_HERC6_SLC1A2_IL1R2_OLAH | HERC6_SLC1A2_OLAH_RETN_MMP8 |
| SIGLEC1_ISG15_HERC6_SLC1A2_OLAH | ISG15_HERC6_SLC1A2_IL1R2_RETN |
| SIGLEC1_ISG15_HERC6_IL1R2_MMP8 | HERC6_SLC1A2_IL1R2_OLAH_MMP8 |
| ISG15_HERC6_SLC1A2_IL1R2_FAM20A | ISG15_HERC6_FAM20A_RETN_MMP8 |
| SIGLEC1_SLC1A2_FAM20A_RETN_MMP8 | ISG15_HERC6_SLC1A2_IL1R2_MMP8 |
| HERC6_SLC1A2_IL1R2_FAM20A_OLAH | ISG15_SLC1A2_FAM20A_OLAH_RETN |
| SIGLEC1_ISG15_FAM20A_OLAH_RETN | ISG15_HERC6_OLAH_RETN_MMP8 |
| SIGLEC1_ISG15_SLC1A2_OLAH_RETN | ISG15_SLC1A2_IL1R2_OLAH_RETN |
| SIGLEC1_HERC6_IL1R2_RETN_MMP8 | ISG15_SLC1A2_IL1R2_OLAH_MMP8 |
| HERC6_IL1R2_FAM20A_RETN_MMP8 | SIGLEC1_ISG15_HERC6_SLC1A2_RETN |
| SIGLEC1_HERC6_SLC1A2_IL1R2_MMP8 | HERC6_SLC1A2_IL1R2_RETN_MMP8 |
| SIGLEC1_SLC1A2_OLAH_RETN_MMP8 | HERC6_SLC1A2_FAM20A_RETN_MMP8 |
| ISG15_SLC1A2_IL1R2_FAM20A_OLAH | SIGLEC1_HERC6_SLC1A2_RETN_MMP8 |
| SIGLEC1_HERC6_SLC1A2_FAM20A_RETN | SIGLEC1_ISG15_HERC6_RETN_MMP8 |
| SIGLEC1_ISG15_HERC6_IL1R2_OLAH | ISG15_HERC6_SLC1A2_FAM20A_MMP8 |
| SIGLEC1_ISG15_HERC6_SLC1A2_IL1R2 | ISG15_SLC1A2_OLAH_RETN_MMP8 |
| ISG15_HERC6_SLC1A2_FAM20A_OLAH | SIGLEC1_ISG15_SLC1A2_RETN_MMP8 |
| SIGLEC1_ISG15_SLC1A2_IL1R2_MMP8 | SIGLEC1_ISG15_HERC6_SLC1A2_MMP8 |
| SIGLEC1_ISG15_IL1R2_OLAH_MMP8 | ISG15_SLC1A2_IL1R2_RETN_MMP8 |
| SIGLEC1_ISG15_SLC1A2_FAM20A_MMP8 | ISG15_IL1R2_OLAH_RETN_MMP8 |
| HERC6_SLC1A2_FAM20A_OLAH_MMP8 | ISG15_FAM20A_OLAH_RETN_MMP8 |
| SLC1A2_FAM20A_OLAH_RETN_MMP8 | SLC1A2_IL1R2_FAM20A_OLAH_MMP8 |
| SLC1A2_IL1R2_OLAH_RETN_MMP8 | ISG15_HERC6_SLC1A2_RETN_MMP8 |
| ISG15_SLC1A2_FAM20A_RETN_MMP8 | SLC1A2_IL1R2_FAM20A_OLAH_RETN |
| IL1R2_FAM20A_OLAH_RETN_MMP8 | SLC1A2_IL1R2_FAM20A_RETN_MMP8 |

Preferably, according to this variant, the measurement step comprises or consists in measuring the expression of a combination of five target genes chosen from the following combinations:
SIGLEC1_HERC6_IL1R2_FAM20A_MMP8, SIGLEC1_IL1R2_FAM20A_OLAH_MMP8; SIGLEC1_ISG15_IL1R2_FAM20A_MMP8 SIGLEC1_IL1R2_FAM20A_RETN_MMP8; SIGLEC1_SLC1A2_IL1R2_FAM20A_MMP8 SIGLEC1_ISG15_HERC6_IL1R2_FAM20A; SIGLEC1_HERC6_IL1R2_FAM20A_OLAH; SIGLEC1_SLC1A2_FAM20A_OLAH_MMP8 and SIGLEC1_ISG15_IL1R2_FAM20A_OLAH.

According to a preferred variant of this embodiment, step (a) comprises or consists in measuring the expression of the target viral gene IFI27, of two other target viral genes chosen from SIGLEC1, HERC6, OAS1 and IFIT1, of the target bacterial gene FAM20A and of another target bacterial gene chosen from OLAH, IL1R2 and MMP8.

According to another preferred variant of this embodiment, step (a) comprises or consists in measuring the expression of a combination of five target genes chosen from those listed in Table 9, and preferably those in this table which have a performance in terms of area under the ROC curve of the model of at least 0.86, at least 0.88, and most particularly, at least 0.90.

According another preferred variant of this embodiment, step (a) comprises or consists in measuring the expression of the target viral genes IFI27 and SIGLEC1, and of the target bacterial genes FAM20A, IL1R2 and MMP8.

According to another particular embodiment, step (a) comprises or consists in measuring the expression of at least six target genes.

According to a variant of this particular embodiment, the measurement step comprises or consists in measuring the expression of the target genes SIGLEC1, ISG15 and HERC6, and of at least three other target genes chosen from IL1R2, SLC1A2, OLAH, FAM20A, RETN and MMP8.

According to another variant of this particular embodiment, the measurement step comprises or consists in measuring the expression of the target genes SIGLEC1, FAM20A and MMP8, and of at least three other target genes chosen from ISG15, HERC6, IL1R2, SLC1A2, OLAH and RETN.

According to another variant of this particular embodiment, the measurement step comprises or consists in measuring the expression of the target genes SIGLEC1, ISG15 and SLC1A2, and of at least three other target genes chosen from HERC6, IL1R2, OLAH, FAM20A, MMP8 and RETN.

According to another variant of this particular embodiment, the measurement step comprises or consists in measuring the expression of the target genes SIGLEC1, and IL1R2, and of at least four other target genes chosen from HERC6, SLC1A2, OLAH, FAM20A, RETN and MMP8.

According to another variant of this particular embodiment, the measurement step comprises or consists in measuring the expression of the target genes SIGLEC1 and HERC6, and of at least four other target genes chosen from ISG15, IL1R2, SLC1A2, OLAH, FAM20A, RETN and MMP8.

According to another variant of this particular embodiment, the measurement step comprises or consists in measuring the expression of the target genes SIGLEC1 and MMP8, and of at least four other target genes chosen from HERC6, ISG15, IL1R2, SLC1A2, OLAH, FAM20A and RETN.

According to another variant of this particular embodiment, the measurement step comprises or consists in measuring the expression of the target genes SIGLEC1 and OLAH, and of at least four other target genes chosen from HERC6, ISG15, IL1R2, SLC1A2, MMP8, FAM20A and RETN.

According to another variant of this particular embodiment, the measurement step comprises or consists in measuring the expression of a combination of six target genes chosen from the following combinations:
SIGLEC1_ISG15_IL1R2_FAM20A_OLAH_MMP8; SIGLEC1_HERC6_IL1R2_FAM20A_OLAH_MMP8; SIGLEC1_ISG15_HERC6_IL1R2_FAM20A_MMP8; SIGLEC1_SLC1A2_IL1R2_FAM20A_OLAH_MMP8; SIGLEC1_HERC6_IL1R2_FAM20A_RETN_MMP8; SIGLEC1_ISG15_IL1R2_FAM20A_RETN_MMP8; SIGLEC1_IL1R2_FAM20A_OLAH_RETN_MMP8; SIGLEC1_ISG15_SLC1A2_IL1R2_FAM20A_MMP8; SIGLEC1_HERC6_SLC1A2_IL1R2_FAM20A_MMP8; SIGLEC1_SLC1A2_IL1R2_FAM20A_RETN_MMP8; SIGLEC1_ISG15_SLC1A2_FAM20A_OLAH_MMP8; SIGLEC1_ISG15_HERC6_IL1R2_FAM20A_OLAH; SIGLEC1_HERC6_SLC1A2_FAM20A_OLAH_MMP8; SIGLEC1_ISG15_IL1R2_FAM20A_OLAH_RETN; SIGLEC1_HERC6_SLC1A2_IL1R2_FAM20A_OLAH; SIGLEC1_SLC1A2_IL1R2_FAM20A_OLAH_RETN; SIGLEC1_SLC1A2_FAM20A_OLAH_RETN_MMP8; SIGLEC1_ISG15_SLC1A2_IL1R2_FAM20A_OLAH; SIGLEC1_ISG15_HERC6 IL1R2_FAM20A_RETN; SIGLEC1_HERC6_IL1R2_FAM20A_OLAH_RETN; SIGLEC1_ISG15_HERC6_SLC1A2_IL1R2_FAM20A; SIGLEC1_ISG15_SLC1A2_IL1R2_FAM20A_RETN; SIGLEC1_HERC6_FAM20A_OLAH_RETN_MMP8; SIGLEC1_ISG15_HERC6_FAM20A_OLAH_MMP8; SIGLEC1_ISG15_HERC6_SLC1A2_FAM20A_OLAH; SIGLEC1_HERC6_SLC1A2_IL1R2_FAM20A_RETN; SIGLEC1_ISG15_FAM20A_OLAH_RETN_MMP8; SIGLEC1_ISG15_SLC1A2_FAM20A_OLAH_RETN; ISG15_HERC6_IL1R2_FAM20A_OLAH_MMP8; SIGLEC1_ISG15_HERC6_FAM20A_RETN_MMP8; ISG15_HERC6_IL1R2_FAM20A_RETN_MMP8; SIGLEC1_HERC6_SLC1A2_FAM20A_OLAH_RETN; ISG15_HERC6_IL1R2_FAM20A_OLAH_RETN; SIGLEC1_ISG15_HERC6_FAM20A_OLAH_RETN; SIGLEC1_ISG15_SLC1A2_IL1R2_OLAH_MMP8; SIGLEC1_HERC6_SLC1A2_IL1R2_OLAH_MMP8; SIGLEC1_ISG15_HERC6_SLC1A2_OLAH_MMP8; HERC6_IL1R2_FAM20A_OLAH_RETN_MMP8; SIGLEC1_HERC6_SLC1A2_FAM20A_RETN_MMP8; ISG15_HERC6_SLC1A2_IL1R2_FAM20A_OLAH; SIGLEC1_ISG15_HERC6_SLC1A2_IL1R2_OLAH; SIGLEC1_SLC1A2_IL1R2_OLAH_RETN_MMP8; SIGLEC1_ISG15_SLC1A2_FAM20A_RETN_MMP8; SIGLEC1_ISG15_SLC1A2_IL1R2_OLAH_RETN; HERC6_SLC1A2_IL1R2_FAM20A_OLAH_MMP8; SIGLEC1_ISG15_HERC6_SLC1A2_FAM20A_RETN; ISG15_HERC6_SLC1A2_IL1R2_FAM20A_RETN; SIGLEC1_ISG15_HERC6_IL1R2_RETN_MMP8; SIGLEC1_ISG15_SLC1A2_OLAH_RETN_MMP8; SIGLEC1_HERC6_IL1R2_OLAH_RETN_MMP8; SIGLEC1_ISG15_HERC6_IL1R2_OLAH_MMP8; SIGLEC1_HERC6_SLC1A2_OLAH_RETN_MMP8; HERC6_SLC1A2_IL1R2_FAM20A_OLAH_RETN; ISG15_HERC6_SLC1A2_IL1R2_FAM20A_MMP8; SIGLEC1_ISG15_HERC6_SLC1A2_IL1R2_MMP8; SIGLEC1_HERC6_SLC1A2_IL1R2_OLAH_RETN; ISG15_HERC6_FAM20A_OLAH_RETN_MMP8; SIGLEC1_ISG15_HERC6_SLC1A2_OLAH_RETN; SIGLEC1_HERC6_SLC1A2_IL1R2_RETN_MMP8; ISG15_HERC6_SLC1A2_FAM20A_OLAH_RETN; HERC6_SLC1A2_FAM20A_OLAH_RETN_MMP8; SIGLEC1_ISG15_HERC6_SLC1A2_FAM20A_MMP8; ISG15_HERC6_SLC1A2_FAM20A_OLAH_MMP8; SIGLEC1_ISG15_SLC1A2_IL1R2_RETN_MMP8; HERC6_SLC1A2_IL1R2_FAM20A_RETN_MMP8; SIGLEC1_ISG15_HERC6_SLC1A2_IL1R2_RETN; SIGLEC1_ISG15_IL1R2_OLAH_RETN_MMP8; ISG15_SLC1A2_IL1R2_FAM20A_OLAH_MMP8; SIGLEC1_ISG15_HERC6_IL1R2_OLAH_RETN; ISG15_SLC1A2_IL1R2_FAM20A_OLAH_RETN; SIGLEC1_ISG15_HERC6_OLAH_RETN_MMP8; ISG15_HERC6_IL1R2_OLAH_RETN_MMP8; ISG15_HERC6_SLC1A2_IL1R2_OLAH_RETN; ISG15_HERC6_SLC1A2_OLAH_RETN_MMP8; ISG15_HERC6_SLC1A2_IL1R2_OLAH_MMP8; HERC6_SLC1A2_IL1R2_OLAH_RETN_MMP8; ISG15_SLC1A2_IL1R2_FAM20A_RETN_MMP8; ISG15_IL1R2_FAM20A_OLAH_RETN_MMP8; ISG15_HERC6_SLC1A2_FAM20A_RETN_MMP8; ISG15_HERC6_SLC1A2_IL1R2_RETN_MMP8; ISG15_SLC1A2_FAM20A_OLAH_RETN_MMP8; SIGLEC1_ISG15_HERC6_SLC1A2_RETN_MMP8; ISG15_SLC1A2_IL1R2_OLAH_RETN_MMP8; and SLC1A2_IL1R2_FAM20A_OLAH_RETN_MMP8.

Preferably, according to this variant, the measurement step comprises or consists in measuring the expression of a combination of six target genes chosen from the following combinations:
SIGLEC1_ISG15_IL1R2_FAM20A_OLAH_MMP8; SIGLEC1_HERC6_IL1R2_FAM20A_OLAH_MMP8; SIGLEC1_ISG15_HERC6_IL1R2_FAM20A_MMP8; SIGLEC1_SLC1A2_IL1R2_FAM20A_OLAH_MMP8; SIGLEC1_HERC6_IL1R2_FAM20A_RETN_MMP8; SIGLEC1_ISG15_IL1R2_FAM20A_RETN_MMP8; and SIGLEC1_IL1R2_FAM20A_OLAH_RETN_MMP8

According to a preferred variant of this embodiment, step (a) comprises or consists in measuring the expression of the target viral genes IFI27 and SIGLEC1, of another target viral gene chosen from IFIT1, HERC6, ISG15 and ISG15, of the target bacterial genes FAM20A and MMP8, and of another target bacterial gene chosen from OLAH and IL1R2.

According to another preferred variant of this embodiment, step (a) comprises or consists in measuring the expression of a combination of six target genes chosen from those listed in Table 10, and preferably those in this table which have a performance in terms of area under the ROC curve of the model of at least 0.86, at least 0.88, and most particularly, at least 0.90.

According to another particular embodiment, step (a) comprises or consists in measuring the expression of at least seven target genes.

According to a variant of this particular embodiment, step (a) comprises or consists in measuring the expression of the target gene SIGLEC1, and of six other target genes chosen from ISG15, HERC6, OLAH, FAM20A, IL1R2, MMP8, RETN and SLC1A2.

According to another variant of this particular embodiment, step (a) comprises or consists in measuring the expression of the target gene MMP8, and of six other target genes chosen from ISG15, HERC6, SLC1A2, IL1R2, FAM20A, OLAH, RETN and SIGLEC1.

According to another variant of this particular embodiment, the measurement step comprises or consists in measuring the expression of the target genes SIGLEC1 and MMP8 and of at least four other target genes chosen from ISG15, HERC6, IL1R2, SLC1A2, OLAH, FAM20A and RETN, preferably from ISG15, HERC6, IL1R2, OLAH, FAM20A and RETN.

According to another variant of this particular embodiment, the measurement step comprises or consists in measuring the expression of the target genes SIGLEC1, and ISG15, and of five other target genes chosen from HERC6, SLC1A2, IL1R2, FAM20A, OLAH, RETN and MMP8.

According to another variant of this particular embodiment, the measurement step comprises or consists in measuring the expression of the target genes SIGLEC1, ISG15 and HERC6, and of four other target genes chosen from SLC1A2, IL1R2, FAM20A, OLAH, RETN and MMP8.

According to another variant of this particular embodiment, the measurement step comprises or consists in measuring the expression of the target genes SIGLEC1, FAM20A and MMP8 and of at least three other target genes chosen from ISG15, HERC6, IL1R2, SLC1A2, OLAH and RETN, preferably from ISG15, HERC6, IL1R2, OLAH and RETN.

According to another variant of this particular embodiment, the measurement step comprises or consists in measuring the expression of the target genes SIGLEC1, FAM20A, IL1R2 and MMP8 and of at least two other target genes chosen from ISG15, HERC6, SLC1A2, OLAH and RETN, preferably from ISG15, HERC6, OLAH and RETN.

According to another variant of this particular embodiment, the measurement step comprises or consists in measuring the expression of seven target genes chosen from the following combinations:
SIGLEC1_ISG15_HERC6_IL1R2_FAM20A_OLAH_MMP8; SIGLEC1_ISG15_SLC1A2_IL1R2_FAM20A_OLAH_MMP8; IGLEC1_HERC6_SLC1A2_IL1R2_FAM20A_OLAH_MMP8; SIGLEC1_ISG15_IL1R2_FAM20A_OLAH_RETN_MMP8; SIGLEC1_ISG15_HERC6_IL1R2_FAM20A_RETN_MMP8; SIGLEC1_HERC6_IL1R2_FAM20A_OLAH_RETN_MMP8; SIGLEC1_ISG15_HERC6_SLC1A2_IL1R2_FAM20A_MMP8; SIGLEC1_ISG15_SLC1A2_IL1R2_FAM20A_RETN_MMP8; SIGLEC1_SLC1A2_IL1R2_FAM20A_OLAH_RETN_MMP8; SIGLEC1_HERC6_SLC1A2_IL1R2_FAM20A_RETN_MMP8; SIGLEC1_ISG15_HERC6_SLC1A2_FAM20A_OLAH_MMP8; SIGLEC1_ISG15_SLC1A2_FAM20A_OLAH_RETN_MMP8; SIGLEC1_ISG15_SLC1A2_IL1R2_FAM20A_OLAH_RETN; SIGLEC1_HERC6_SLC1A2_FAM20A_OLAH_RETN_MMP8; SIGLEC1_ISG15_HERC6_IL1R2_FAM20A_OLAH_RETN; SIGLEC1_ISG15_HERC6_SLC1A2_IL1R2_FAM20A_OLAH; SIGLEC1_ISG15_HERC6_FAM20A_OLAH_RETN_MMP8; SIGLEC1_HERC6_SLC1A2_IL1R2_FAM20A_OLAH_RETN; SIGLEC1_ISG15_HERC6_SLC1A2_IL1R2_FAM20A_RETN; SIGLEC1_ISG15_HERC6_SLC1A2_FAM20A_OLAH_RETN; ISG15_HERC6_IL1R2_FAM20A_OLAH_RETN_MMP8; SIGLEC1_ISG15_HERC6_SLC1A2_FAM20A_RETN_MMP8; SIGLEC1_ISG15_HERC6_SLC1A2_IL1R2_OLAH_MMP8; SIGLEC1_ISG15_SLC1A2_IL1R2_OLAH_RETN_MMP8; ISG15_HERC6_SLC1A2_IL1R2_FAM20A_RETN_MMP8; ISG15_HERC6_SLC1A2_IL1R2_FAM20A_OLAH_MMP8; SIGLEC1_HERC6_SLC1A2_IL1R2_OLAH_RETN_MMP8; ISG15_HERC6_SLC1A2_IL1R2_FAM20A_OLAH_RETN; HERC6_SLC1A2_IL1R2_FAM20A_OLAH_RETN_MMP8; SIGLEC1_ISG15_HERC6_IL1R2_OLAH_RETN_MMP8; SIGLEC1_ISG15_HERC6_SLC1A2_OLAH_RETN_MMP8; ISG15_HERC6_SLC1A2_FAM20A_OLAH_RETN_MMP8; SIGLEC1_ISG15_HERC6_SLC1A2_IL1R2_OLAH_RETN; SIGLEC1_ISG15_HERC6_SLC1A2_IL1R2_RETN_MMP8; ISG15_SLC1A2_IL1R2_FAM20A_OLAH_RETN_MMP8; and ISG15_HERC6_SLC1A2_IL1R2_OLAH_RETN_MMP8.

Preferably, according to this variant, the measurement step comprises or consists in measuring the expression of seven target genes chosen from the following combinations:
SIGLEC1_ISG15_SLC1A2_IL1R2_FAM20A_OLAH_MMP8; IGLEC1_HERC6_SLC1A2_IL1R2_FAM20A_OLAH_MMP8; SIGLEC1_ISG15_IL1R2_FAM20A_OLAH_RETN_MMP8; SIGLEC1_ISG15_HERC6_IL1R2_FAM20A_RETN_MMP8; SIGLEC1_HERC6_IL1R2_FAM20A_OLAH_RETN_MMP8; SIGLEC1_ISG15_HERC6_SLC1A2_IL1R2_FAM20A_MMP8; and SIGLEC1_ISG15_SLC1A2_IL1R2_FAM20A_RETN_MMP8.

According to a preferred variant of this particular embodiment, step (a) comprises or consists in measuring the expression of the target viral genes IFI27 and SIGLEC1, of two other target viral gene chosen from IFIT1, OAS1, HERC6 and ISG15, of the target bacterial genes FAM20A and MMP8, and of another target bacterial gene chosen from OLAH and IL1R2.

According to another preferred variant of this particular embodiment, step (a) comprises or consists in measuring the expression of a combination of seven target genes chosen from those listed in Table 11, and preferably those in this table which have a performance in terms of area under the ROC curve of the model of at least 0.86, at least 0.88, and most particularly, at least 0.90.

According to another particular embodiment, step (a) comprises or consists in measuring the expression of at least eight target genes.

According to a variant of this particular embodiment, the measurement step comprises or consists in measuring the expression of SIGLEC1 and ISG15, and of at least six other target genes chosen from HERC6, SLC1A2, IL1R2, FAM20A, OLAH, RETN and MMP8.

According to another variant of this particular embodiment, the measurement step comprises or consists in measuring the expression of SIGLEC1 and MMP8, and of at least six other target genes chosen from HERC6, ISG15, SLC1A2, IL1R2, FAM20A, OLAH and RETN.

According to another variant of this particular embodiment, the measurement step comprises or consists in measuring the expression of SIGLEC1, MMP8 and ISG15, and of at least five other target genes chosen from HERC6, SLC1A2, IL1R2, FAM20A, OLAH and RETN.

According to another variant of this particular embodiment, the measurement step comprises or consists in measuring the expression of a combination of eight target genes chosen from the following combinations:
SIGLEC1_ISG15_HERC6_IL1R2_FAM20A_OLAH_RETN_MMP8; SIGLEC1_ISG15_HERC6_SLC1A2_IL1R2_FAM20A_OLAH_MMP8; SIGLEC1_ISG15_SLC1A2_IL1R2_FAM20A_OLAH_RETN_MMP8; SIGLEC1_ISG15_HERC6_SLC1A2_IL1R2_FAM20A_RETN_MMP8; SIGLEC1_HERC6_SLC1A2_IL1R2_FAM20A_OLAH_RETN_MMP8; SIGLEC1_ISG15_HERC6_SLC1A2_FAM20A_OLAH_RETN_MMP8; SIGLEC1_ISG15_HERC6_SLC1A2_IL1R2_FAM20A_OLAH_RETN; ISG15_HERC6_SLC1A2_IL1R2_FAM20A_OLAH_RETN_MMP8, and SIGLEC1_ISG15_HERC6_SLC1A2_IL1R2_OLAH_RETN_MMP8.

According to another variant of this particular embodiment, step (a) comprises or consists in measuring the expression of a combination of eight target genes chosen from those listed in Table 12, and preferably those which have a performance in terms of area under the ROC curve of the model of at least 0.87, at least 0.88, and most particularly, at least 0.89.

According to another particular embodiment, the method comprises a step of measuring the variation in the expression of at least nine target genes.

According to another variant of this particular embodiment, step (a) comprises or consists in measuring the expression of a combination of nine target genes chosen from those listed in Table 13, and preferably those which have a performance in terms of area under the ROC curve of the model of at least 0.87, at least 0.88, and most particularly, at least 0.89.

According to another particular embodiment, the method comprises a step of measuring the variation in the expression of ten target genes.

According to another variant of this particular embodiment, step (a) comprises or consists in measuring the expression of a combination of ten target genes chosen from those listed in Table 14, and preferably those which have a performance in terms of area under the ROC curve of the model of at least 0.87, at least 0.88, and most particularly, at least 0.89.

According to another particular embodiment, the method comprises a step of measuring the variation in the expression of at least eleven target genes.

According to another variant of this particular embodiment, step (a) comprises or consists in measuring the expression of a combination of eleven target genes chosen from those listed in Table 15, and preferably those which have a performance in terms of area under the ROC curve of the model of at least at least 0.86, and most particularly, at least 0.87.

According to another particular embodiment, the method comprises a step of measuring the variation in the expression of at least twelve target genes.

According to another variant of this particular embodiment, step (a) comprises or consists in measuring the expression of a combination of twelve target genes chosen from those listed in Table 16, and preferably those which have a performance in terms of area under the ROC curve of the model of at least at least 0.86, and most particularly, at least 0.87.

According to another particular embodiment, the method comprises a step of measuring the variation in the expression of at least thirteen target genes.

According to another variant of this particular embodiment, step (a) comprises or consists in measuring the expression of a combination of thirteen target genes chosen from those listed in Table 17.

In the present description, the term “biological sample” refers to any sample originating from a subject, which may be of different natures, such as blood or derivatives thereof, sputum, urine, stools, skin, cerebrospinal fluid, bronchoalveolar lavage fluid, abdominal cavity puncture fluid, saliva, gastric secretions, sperm, seminal fluid, tears, spinal cord, trigeminal nerve ganglion, adipose tissue, lymphoid tissue, placental tissue, gastrointestinal tract tissue, genital tract tissue, or central nervous system tissue.

The expression “reference biological sample”, or “control sample”, refers to the biological sample from which the levels of expression of the target genes, and optionally those of additional genes, in the biological sample to be tested are compared. The reference biological sample is of the same nature as the biological sample to be tested, or at least of a compatible nature to constitute a reference for determining the expression level of target genes. This sample is taken from a subject having a bacterial or viral infection, or from a pool of biological samples all taken from patients having an infection of the same nature, i.e. viral or bacterial.

According to a particular embodiment, the reference biological sample is calibrated to contain the amount of transcripts of at least two target genes, and optionally of one or more additional genes, corresponding to the quantity or concentration representative of the level of expression measured in a pool of samples from subjects presenting with a bacterial or viral infection. In other words, the reference biological sample is calibrated to contain the average amount of transcripts of said target genes obtained from a pool of samples from subjects having a bacterial infection or from subjects having a viral infection.

In particular, the biological sample may be a biological fluid, such as a blood sample or a blood-derived sample, which may notably be chosen from whole blood (as collected from a vein, i.e. containing white and red blood cells, platelets and plasma), plasma, serum, and any types of cells extracted from the blood, for instance peripheral blood mononuclear cells (PBMCs, containing B lymphocytes, T lymphocytes, NK cells, dendritic cells and monocytes), B and T cell subsets, purified monocytes, or neutrophils.

According to a preferred embodiment, the biological sample used in the method according to the invention is a blood sample, preferably a whole blood sample.

According to another particular embodiment, the subject is a patient in a hospital, preferably in the emergency department, the resuscitation department, the intensive care unit (ICU) or the ongoing care unit, most particularly a patient in the emergency department.

According to a preferred embodiment, the subject is a patient less than 6 years old, preferably less than 4 years old, and more preferably less than 2 years old. According to this embodiment, the patient may be in the emergency department, notably in a pediatric emergency.

According to a particular embodiment, the method allows the nature of the infection to be identified with a sensitivity of at least 80%, 85%, 90% or even 95%. Sensitivity corresponds to the probability of correctly identifying the viral or bacterial nature of the infection when the subject is infected, and may be defined by the following formula:

$$Sensitivity = \frac{number of true positives}{number of true positives + number of false negatives}$$

According to a particular embodiment, the method allows the nature of the infection to be identified with a specificity of at least 80%, 85%, 90% or even at least 95%.

Specificity corresponds to the probability of erroneously identifying the viral or bacterial nature of the infection. In other words, to identify the presence of a viral infection whereas it is a bacterial infection, or vice versa. Specificity may be defined by the following formula:

$$Specificity = \frac{number of true negatives}{number of true negatives + number of false positives}$$

The sensitivity and specificity of a diagnostic test, such as the method according to the present invention, may also be summarized by means of an ROC (Receiver Operating Characteristics) curve, in particular the area under the ROC curve, which is well known to those skilled in the art.

Thus, the method according to the present description allows identification of the viral or bacterial nature of an infection with a performance reflected by an area under the ROC curve of at least 0.85, at least 0.86, at least 0.87, at least 0.88, at least 0.89, and most particularly of at least 0.90 for the best-performing transcriptomic signatures.

Although the performance of the method according to the invention is entirely satisfactory, in certain situations, measurement of the expression variation of target genes may be supplemented by measurement of the expression variation of additional genes.

Thus, the method according to the invention, in all its embodiments, may also comprise measurement of the variation of at least one additional gene chosen from the following genes: PI3, EBI3, ADGRE1 and S100P.

These additional genes are also known to those skilled in the art, and the chromosomal positions are given in Table 5 below:

TABLE 5

| Additional | Chromosomal location | |
|---|---|---|
| biomarker | (GRCh38/hg38) | English name |
| PI3 | Chr20:45174902-45176544 | Peptidase Inhibitor 3 |
| EBI3 | Chr19:4229523-4237528 | Epstein-Barr Virus Induced 3 |
| ADGRE1 | Chr19:6887566-6940459 | Adhesion G Protein-Coupled Receptor E1 |
| S100P | Chr4:6693878-6697170 | S100 Calcium Binding Protein P |

Similarly to the preceding, for a given additional gene, the reference value corresponds to a level of expression of transcripts of said gene obtained from a reference biological sample from a subject presenting with an infection. Advantageously, and notably for the variants defined hereinbelow, the reference value for a given additional gene corresponds to the mean mRNA transcript level of said gene obtained from reference biological samples from a population of subjects presenting with an infection.

According to a first variant, the reference value for each additional gene is determined on the basis of a reference biological sample from a subject suffering from a bacterial infection. Thus, the presence of a viral infection may be concluded when comparison of the level of expression of the additional genes at mRNA transcript level relative to the respective reference values reveals at least one expression variation chosen from:
an under-expression of PI3,
an under-expression of EBI3,
an under-expression of ADGRE1, and
an under-expression of S100P.

According to a second variant, the reference value for each additional gene is determined on the basis of a reference biological sample from subjects suffering from a viral infection. Thus, the presence of a bacterial infection may be concluded when comparison of the level of expression of the additional genes at mRNA transcript level relative to the respective reference values reveals at least one variation in expression chosen from:
an over-expression of PI3,
an over-expression of EBI3,
an over-expression of ADGRE1, and
an over-expression of S100P.

According to a particular embodiment, the method according to the invention comprises measurement of the variation in expression of all the following target genes and additional genes: SIGLEC1, ISG15, HERC6, SLC1A2, IL1R2, FAM20A, OLAH, RETN, MMP8, RSAD2, IFI27, OAS1, IFIT1, IFI44L, PI3, EBI3, ADGRE1 and S100P.

Conclusion as to the viral or bacterial nature of the infection is made as defined previously on the basis of over- or under-expression of said genes as a function of the determined reference values.

Thus, according to a particular embodiment, the method comprises the steps of:
obtaining a biological sample of blood, preferably whole blood, from a subject having an infection,
placing said biological sample in contact with reagents specific for the expression products of one or more viral genes as described in the present description, and of at least one target bacterial gene chosen from OLAH, FAM20A, IL1R2, MMP8, RETN and SLC1A2, and optionally of one or more additional genes chosen from PI3, EBI3, ADGRE1 and S100P,
measuring the expression of said target genes, and optionally of the additional gene(s).

According to a preferred embodiment, the method comprises the steps of:
obtaining a biological sample of blood, preferably whole blood, from a subject having an infection,
placing said biological sample in contact with reagents specific for the expression products of at least one target viral gene chosen from SIGLEC1, ISG15, HERC6, RSAD2, IFI27, OAS1, IFIT1, IFI44L, and at least one target bacterial gene chosen from SLC1A2, IL1R2, FAM20A, OLAH, RETN and MMP8, and optionally of one or more additional genes chosen from PI3, EBI3, ADGRE1 and S100P,
measuring the expression of said target genes, and optionally of the additional gene(s).

Another subject of the invention relates to a kit for in vitro or ex vivo measurement of the expression of at least one target viral gene chosen from SIGLEC1, ISG15, HERC6, RSAD2, IFI27, OAS1, IFIT1, IFI44L, and at least one target bacterial gene chosen from OLAH, FAM20A, IL1R2, MMP8, RETN and SLC1A2, said kit comprising reagents specific for the expression products of said target genes.

According to a particular embodiment, the kit comprises reagents specific for the expression products of at least three target genes, four target genes, five target genes, six target genes, seven target genes, eight target genes or all the target genes.

Most particularly, the kit allows the method for according to the invention to be performed. The kit according to the invention may thus be advantageously used for determining the viral or bacterial nature of an infection.

Consequently, all the particular and preferred embodiments described for the method according to the invention, notably the combinations of target genes whose expression is measured, also apply to the kit.

Specific reagents for measuring gene expression are known to those skilled in the art and, according to a particular embodiment, include amplification primers and/or hybridization probes.

Such reagents allow quantitative determination of the transcript level of said selected gene in the biological test sample. As explained previously, the transcript level determined may not correspond directly to the amount of transcripts present in the biological sample, but may be a derived value representative of the amount of transcripts. Specifically, in a conventional manner, quantitative determination may include a step of amplifying and/or normalizing and/or calculating a ratio, etc.

The term “kit” means a set of products and/or tools to be used together to obtain, in particular, determination of the transcript level of target genes defined in the context of the invention. The necessary reagents may or may not be collated in the same kit or device.

The term “primer” or “amplification primer” means a nucleotide fragment which may consist of 5 to 100 nucleotides, preferably 15 to 30 nucleotides, and having hybridization specificity with a target nucleotide sequence under conditions determined for the initiation of enzymatic polymerization, for example in a reaction for the enzymatic amplification of the target nucleotide sequence. Generally, use is made of “primer pairs” consisting of two primers. When it is desired to amplify several different biomarkers (e.g. genes or mRNAs of said genes), several different pairs of primers are preferably used, each preferentially having the ability to hybridize specifically with a different biomarker.

A person skilled in the art is entirely capable of determining the nucleotide sequences of the primers, so as to allow amplification of the transcripts, from the sequence of a gene and notably from the sequence of the corresponding transcripts, for example those listed in Table 2.

According to a particular embodiment, primers are present in the kit, said primers comprising, or consisting of, a nucleotide sequence complementary to at least a portion of a sequence of the transcripts as presented in Table 2 above.

The term “probe” or “hybridization probe” means a nucleotide fragment typically consisting of 5 to 100 nucleotides, preferably 15 to 90 nucleotides, even more preferably 15 to 35 nucleotides, having hybridization specificity under conditions determined for forming a hybridization complex with a target nucleotide sequence. The probe also includes a reporter (such as a fluorophore, an enzyme or any other detection system) which will enable the detection of the target nucleotide sequence. In the present invention, the target nucleotide sequence may be a nucleotide sequence contained in a messenger RNA (mRNA) or a nucleotide sequence contained in a complementary DNA (cDNA) obtained by reverse transcription of said mRNA. When it is desired to target several different biomarkers (e.g. genes or mRNAs of said genes), several different probes are preferably used, each preferentially having the ability to hybridize specifically with a different biomarker.

The term “hybridization” means the process during which, under suitable conditions, two nucleotide fragments, for instance a hybridization probe and a target nucleotide fragment, having sufficiently complementary sequences, are able to form a double strand with stable and specific hydrogen bonds.

A nucleotide fragment which is “able to hybridize” with a polynucleotide is a fragment which can hybridize with said polynucleotide under hybridization conditions, which can be determined in each case in a known manner. The hybridization conditions are determined by stringency, i.e. the strictness of the operating conditions. Hybridization is proportionately more specific the higher the stringency levels at which it is performed.

The stringency is notably defined on the basis of the base composition of a probe/target duplex, and also by the degree of mismatch between two nucleic acids. The stringency can also be based on the reaction parameters, such as the concentration and type of the ionic species present in the hybridization solution, the nature and the concentration of denaturing agents, and/or the hybridization temperature. The stringency of the conditions under which a hybridization reaction must be performed will chiefly depend on the hybridization probes used. All this information is well known and the appropriate conditions can be determined by a person skilled in the art.

In general, depending on the length of the hybridization probes used, the temperature for the hybridization reaction is between about 20 and 70° C., in particular between 35 and 65° C. in a saline solution at a concentration of about 0.5 to 1 M. A step of detecting the hybridization reaction is subsequently performed.

According to a preferred embodiment, the kit comprises a control sample calibrated to contain the amount of transcripts of at least two target genes, and optionally of one or more additional genes, corresponding to the amount or concentration representative of the expression level measured in a pool of samples from subjects presenting with a bacterial infection and/or a control sample calibrated to contain the amount of transcripts of at least two target genes, and optionally one or more additional genes, corresponding to the amount or concentration representative of the level of expression measured in a pool of samples from subjects presenting with a viral infection.

In other words, the control sample is calibrated to contain the average amount of transcripts of said target genes obtained from a pool of samples from subjects presenting with a bacterial infection or from subjects presenting with a viral infection.

According to one embodiment, the reagents specific to the expression of target genes, more precisely, amplification products and/or detection products, for instance primers or probes, may be bound to the same solid support. The kit may thus comprise a solid support comprising one or more oligonucleotides suitable for determining the transcript level of the or each target gene, or even one or more oligonucleotides suitable for determining the transcript level of the or each selected housekeeping gene, or even one or more oligonucleotides suitable for detecting at least one target gene, as described previously. Such solid supports are well known to those skilled in the art, and are notably described in patent applications WO 2008/140568 and WO 2017/093672, to which reference may be made for further details.

The kit, in all its embodiments, may also further comprise reagents specific to the expression products of one or more additional genes chosen from PI3, EBI3, ADGRE1 and S100P and combinations thereof.

In the same manner as defined previously, the kit may also comprise a threshold value for the expression level of the additional genes and/or of the (positive or negative) control samples of said additional genes.

The kit, in all its embodiments, may also further comprise reagents specific to the expression products of one or more housekeeping genes. Here again, the kit according to the invention may comprise reagents specific to the expression products of each selected housekeeping gene.

According to a particular embodiment, the kit is in the form of a consumable integrating nucleic acid purification, multiplex nested PCR and detection in a single consumable in the form of a FilmArray® cassette or pouch. Such a consumable is advantageously intended for use with BioFire® FilmArray® V2.0 and Torch systems.

Another subject relates to the use of a kit as defined previously for determining the viral or bacterial nature of an infection in a subject.

Another subject of the present description relates to a method comprising the quantitative measurement, notably by RT-qPCR, of the mRNAs of at least one target viral gene chosen from IFI27, SIGLEC1, ISG15, HERC6, RSAD2, OAS1, IFIT1, IFI44L, and at least one target bacterial gene chosen from OLAH, FAM20A, IL1R2, MMP8, RETN and SLC1A2, and optionally one or more additional genes as defined previously, in a biological blood sample from a subject presenting with an infection.

The description also relates to methods for determining the viral or bacterial nature of an infection in an infected subject as defined previously, which also comprise a step of treating the viral or bacterial infection.

According to a particular embodiment, when a viral infection is identified, the treatment comprises the administration of at least one antiviral agent. A person skilled in the art is able to choose the most suitable antiviral agent from among the known agents. By way of example, the antiviral agent may be chosen from the following agents: amantadine, rimantadine, ritonavir, cobicistat, interferon alfa-2b/ribavirin, ombitasvir/paritaprevir/ritonavir, peginterferon alfa-2a, peginterferon alfa-2b, maraviroc, raltegravir, dolutegravir, elvitegravir, sofosbuvir, enfuvirtide, foscarnet, fomivirsen, zanamivir, oseltamivir, peramivir, nevirapine, etravirine, efavirenz, rilpivirine, delavirdine, nevirapine, daclatasvir, entacavir, lamivudine, adefovir, didanosine, tenofovir, abacavir, lamivudine, zidovudine, stavudine, emtricitabine, zalcitabine, telbivudine, didanosine, boceprevir, simeprevir, telaprevir, lopinavir, fosamprenavir, darunavir, ritonavir, tipranavir, atazanavir, nelfinavir, amprenavir, indinavir, saquinavir, ribavirin, valacyclovir, famciclovir, acyclovir, ganciclovir, valganciclovir and cidofovir.

According to a particular embodiment, when a viral infection is identified, the treatment comprises the administration of at least one antibiotic. A person skilled in the art is able to choose the most suitable antibiotic from among the known antibiotics. By way of example, the antibiotic may be chosen from the following antibiotics: erythromycin, clindamucin, gentamicin, tetracycline, amoxicillin, amikacin, aztreonam, chloramphenicol, ceftazidime, clindamycin, cefalotin, ciprofloxacin, colistin, cefotetan, cefotaxime, erythromycin, fusidic acid, fosfomycin, cefoxitin, furans, gentamicin, imipenem, kanamycin, lincomycin, cefamandole, minocycline, latamoxef, metronidazole, nalidixic acid, netilmicin, oxacillin, benzylpenicillin, pefloxacin, piperacillin, pristinamycin, rifampin, spiramycin, sulfonamides, streptomycin, trimethoprim, sulfametoxazole, tetracycline, teicoplanin, ticarcillin, tobramycin, trimethoprim, vancomycin, meclocycline, sulfacetamide, ceftobiprole, ceftaroline, dalbavancin, daptomycin, linezolid, mupirocin, oritavancin, telavancin, tigecycline, vancomycin, aminoglycosides, carbapenems, ceftazidime, cefepime, fluorquinolones, piperacillin/tazobactam, ticarcillin/clavulanic acid, linezolid, streptogramins, daptomycin, amikacin, kanamycin, neomycin, netilmicin, tobramycin, paromomycin, spectinomycin, geldanamycin, herbimycin and rifaximin.

The present invention is illustrated, in a nonlimiting manner, by means of the examples that follow.

### EXAMPLES

### 1. Materials and Method

### 1.1 Clinical Features of the Patients & Samples

Clinical samples of whole blood were collected in PAXgeneR tubes during the patient's admission to the emergency department (DO), following the manufacturer's recommendations. A total of 191 samples were collected, broken down as follows: 68 samples from a characterized bacterial infection and 123 samples from a characterized viral infection.

The source and distribution of the samples are collated below:
Patients: Female-82 (42.9%); Male 109 (57.1%)
Mean age: 3.4 years
Bacterial infection (GRAM−), n(%): 28 (14.6)
Bacterial infection (GRAM+), n(%): 24 (12.6)
Probable bacterial infection, n(%): 16 (8.4)
Viral infection, n(%): 123 (64.4)

### 1.2. Biomarker Identification and Validation

An initial selection of transcriptomic biomarkers characteristic of viral and bacterial infections was performed using the Applicant's internal database. Several hundred biomarkers were thus identified.

For each biomarker, several scores were then assigned in order to evaluate a multitude of criteria, notably the capacity for expression based on RNAseq data from sample banks, the ability to discriminate between viral and bacterial infection based on micro-array data from public databases, and the ability to develop primer pairs for amplification in an automated PCR system (FilmArray® for example).

Taking into account the different scores obtained for each biomarker, an overall score was calculated to allow optimal selection and identification of the most promising biomarkers for identifying the nature of the infection.

Then, to finally validate the biomarkers identified, the patient samples were divided into two data groups. The first group, called the “TRAIN” set (n=128), is used to train the learning model on the data, while the second group, called the “TEST” set (n=63), is used for independent performance validation.

To evaluate individual biomarker performance, the area under the ROC curve was calculated with a 95% confidence interval (CI95%). As regards combination performance, analyses were performed with complex machine-learning-based classifiers using R version 3.6.1.

Two methods in particular were used to evaluate the importance of variables. The first is the “explain” function in the FastShap package, derived from game theory. A reward (weight) is given to biomarkers that afford the most in the machine learning model for classifying patients and allowing identification of the nature of the infection (viral or bacterial).

The second method is the “FeatureImp” function in the IML package, which returns the factor by which the model's prediction error increases when a feature is mixed.

Ultimately, the selection allowed the identification of viral biomarkers, notably those involved in the interferon pathway and the MHC class II-mediated antigen presentation pathway, but also bacterial biomarkers, including SLC1A2, IL1R2, FAM20A, OLAH, RETN and MMP8.

Performance was also evaluated and confirmed on two other machine learning models, namely Random Forest and Partial Least Squares-Discriminant (PLS) regression.

### 2. Results

### 2.1 BoxPlot

BoxPlot analysis reveals the over/under-expression of different target genes depending on the nature of the infection. Thus, when comparing target gene expressions between viral and bacterial infections, it is found that the SLC1A2, IL1R2, FAM20A, OLAH, RETN and MMP genes are over-expressed during bacterial infection, whereas the SIGLEC1, ISG15, HERC6, IFI44L, RSAD2, IFI27, OAS1 and IFIT1 genes are over-expressed during viral infection.

### 2.2 Performance Analysis of the Biomarkers According to the Invention

The performance of the target genes used in the method according to the invention to determine the viral or bacterial nature of an infection is shown in the tables below. For each model, the performance was evaluated several times and the AUCs of the model thus correspond to the mean of the different AUCs obtained.

Performance obtained for the identification of the viral or bacterial nature of the infection on the basis of the two-gene combinations:

TABLE 6

| | AUC | | | AUC | CI 95% | | AUC | CI 95% | |
|---|---|---|---|---|---|---|---|---|---|
| Model | (model) | CI 95% | | (Train) | (Train) | | (Test) | (Test) | |
| IFI27_OLAH | 0.910 | 0.902 | 0.918 | 0.916 | 0.859 | 0.974 | 0.937 | 0.876 | 0.997 |
| IFI27_IL1R2 | 0.903 | 0.896 | 0.910 | 0.913 | 0.864 | 0.962 | 0.910 | 0.837 | 0.982 |
| IFI27_RETN | 0.892 | 0.882 | 0.901 | 0.900 | 0.836 | 0.963 | 0.933 | 0.858 | 1 |
| SIGLEC1_IL1R2 | 0.890 | 0.882 | 0.898 | 0.897 | 0.835 | 0.959 | 0.960 | 0.920 | 1 |
| SIGLEC1_FAM20A | 0.888 | 0.878 | 0.897 | 0.890 | 0.817 | 0.963 | 0.955 | 0.893 | 1 |
| HERC6_IL1R2 | 0.885 | 0.877 | 0.893 | 0.895 | 0.836 | 0.954 | 0.927 | 0.865 | 0.990 |
| SIGLEC1_OLAH | 0.882 | 0.873 | 0.890 | 0.890 | 0.821 | 0.959 | 0.949 | 0.900 | 0.998 |
| IFIT1_IL1R2 | 0.880 | 0.871 | 0.888 | 0.890 | 0.824 | 0.956 | 0.953 | 0.909 | 0.998 |
| IFI27_FAM20A | 0.878 | 0.867 | 0.888 | 0.878 | 0.800 | 0.957 | 0.923 | 0.839 | 1 |
| HERC6_OLAH | 0.876 | 0.867 | 0.885 | 0.886 | 0.819 | 0.953 | 0.941 | 0.889 | 0.993 |
| IFI44L_IL1R2 | 0.876 | 0.868 | 0.884 | 0.886 | 0.825 | 0.947 | 0.941 | 0.889 | 0.993 |
| OAS1_IL1R2 | 0.875 | 0.866 | 0.883 | 0.886 | 0.821 | 0.952 | 0.926 | 0.863 | 0.989 |
| ISG15_IL1R2 | 0.874 | 0.866 | 0.882 | 0.885 | 0.824 | 0.945 | 0.933 | 0.865 | 1 |
| RSAD2_IL1R2 | 0.872 | 0.863 | 0.880 | 0.881 | 0.814 | 0.948 | 0.943 | 0.893 | 0.994 |
| OAS1_OLAH | 0.870 | 0.861 | 0.879 | 0.880 | 0.808 | 0.952 | 0.946 | 0.892 | 0.999 |
| SIGLEC1_RETN | 0.869 | 0.859 | 0.879 | 0.875 | 0.797 | 0.953 | 0.948 | 0.898 | 0.997 |
| IFI27_MMP8 | 0.867 | 0.857 | 0.877 | 0.877 | 0.809 | 0.945 | 0.882 | 0.793 | 0.970 |
| HERC6_FAM20A | 0.866 | 0.856 | 0.876 | 0.874 | 0.802 | 0.945 | 0.925 | 0.838 | 1 |
| IFI27_SIGLEC1 | 0.865 | 0.855 | 0.875 | 0.877 | 0.810 | 0.943 | 0.949 | 0.898 | 0.999 |
| IFI44L_OLAH | 0.865 | 0.856 | 0.874 | 0.871 | 0.798 | 0.943 | 0.938 | 0.883 | 0.993 |
| RSAD2_OLAH | 0.865 | 0.855 | 0.874 | 0.875 | 0.802 | 0.947 | 0.950 | 0.901 | 0.999 |
| SLC1A2_OLAH | 0.865 | 0.855 | 0.874 | 0.880 | 0.817 | 0.942 | 0.845 | 0.737 | 0.952 |
| IFIT1_OLAH | 0.864 | 0.854 | 0.874 | 0.873 | 0.798 | 0.947 | 0.949 | 0.901 | 0.997 |
| HERC6_RETN | 0.864 | 0.853 | 0.874 | 0.872 | 0.795 | 0.950 | 0.928 | 0.867 | 0.990 |
| SIGLEC1_SLC1A2 | 0.864 | 0.855 | 0.872 | 0.877 | 0.811 | 0.944 | 0.957 | 0.914 | 0.999 |
| ISG15_OLAH | 0.863 | 0.854 | 0.872 | 0.873 | 0.805 | 0.941 | 0.940 | 0.886 | 0.994 |
| SIGLEC1_MMP8 | 0.858 | 0.848 | 0.868 | 0.863 | 0.786 | 0.940 | 0.926 | 0.866 | 0.987 |
| IFI27_SLC1A2 | 0.857 | 0.847 | 0.867 | 0.863 | 0.785 | 0.940 | 0.895 | 0.808 | 0.981 |
| HERC6_MMP8 | 0.851 | 0.841 | 0.862 | 0.859 | 0.784 | 0.935 | 0.877 | 0.791 | 0.964 |
| IL1R2_OLAH | 0.851 | 0.842 | 0.860 | 0.862 | 0.796 | 0.928 | 0.843 | 0.733 | 0.954 |
| IFI44L_FAM20A | 0.850 | 0.840 | 0.860 | 0.853 | 0.771 | 0.935 | 0.934 | 0.855 | 1 |
| IL1R2_FAM20A | 0.847 | 0.839 | 0.856 | 0.863 | 0.797 | 0.929 | 0.849 | 0.736 | 0.962 |
| SIGLEC1_HERC6 | 0.847 | 0.837 | 0.857 | 0.854 | 0.778 | 0.930 | 0.946 | 0.896 | 0.995 |
| HERC6_SLC1A2 | 0.842 | 0.833 | 0.851 | 0.857 | 0.787 | 0.926 | 0.902 | 0.820 | 0.984 |
| SIGLEC1_ISG15 | 0.842 | 0.832 | 0.852 | 0.849 | 0.772 | 0.927 | 0.945 | 0.895 | 0.994 |
| FAM20A_OLAH | 0.839 | 0.829 | 0.849 | 0.852 | 0.782 | 0.922 | 0.871 | 0.776 | 0.965 |
| ISG15_FAM20A | 0.838 | 0.828 | 0.848 | 0.846 | 0.769 | 0.923 | 0.924 | 0.844 | 1 |
| SLC1A2_IL1R2 | 0.838 | 0.829 | 0.846 | 0.852 | 0.785 | 0.918 | 0.826 | 0.709 | 0.943 |
| ISG15_RETN | 0.836 | 0.827 | 0.846 | 0.851 | 0.777 | 0.926 | 0.949 | 0.896 | 1 |
| OLAH_MMP8 | 0.836 | 0.826 | 0.845 | 0.847 | 0.778 | 0.916 | 0.861 | 0.761 | 0.960 |
| OLAH_RETN | 0.834 | 0.824 | 0.844 | 0.850 | 0.780 | 0.921 | 0.848 | 0.738 | 0.958 |
| ISG15_HERC6 | 0.829 | 0.819 | 0.840 | 0.839 | 0.763 | 0.914 | 0.921 | 0.856 | 0.986 |
| IL1R2_MMP8 | 0.828 | 0.820 | 0.836 | 0.849 | 0.781 | 0.916 | 0.812 | 0.696 | 0.928 |
| IL1R2_RETN | 0.828 | 0.819 | 0.837 | 0.850 | 0.782 | 0.918 | 0.820 | 0.705 | 0.935 |
| ISG15_MMP8 | 0.819 | 0.810 | 0.829 | 0.832 | 0.754 | 0.910 | 0.898 | 0.823 | 0.972 |
| ISG15_SLC1A2 | 0.803 | 0.793 | 0.813 | 0.820 | 0.744 | 0.895 | 0.911 | 0.839 | 0.983 |

Performance obtained for the identification of the viral or bacterial nature of the infection on the basis of the three-gene combinations:

TABLE 7

| | AUC | | | AUC | CI 95% | | AUC | CI 95% | |
|---|---|---|---|---|---|---|---|---|---|
| Model | (model) | CI 95% | | (Train) | (Train) | | (Test) | (Test) | |
| IFI27_OLAH_FAM20A | 0.912 | 0.903 | 0.920 | 0.922 | 0.862 | 0.981 | 0.929 | 0.857 | 1 |
| IFI27_IL1R2_FAM20A | 0.908 | 0.901 | 0.916 | 0.920 | 0.867 | 0.972 | 0.910 | 0.819 | 1 |
| IFI27_OLAH_MMP8 | 0.907 | 0.899 | 0.915 | 0.916 | 0.859 | 0.974 | 0.937 | 0.876 | 0.997 |
| IFI27_HERC6_OLAH | 0.907 | 0.899 | 0.915 | 0.919 | 0.862 | 0.975 | 0.942 | 0.888 | 0.997 |
| IFI27_IL1R2_OLAH | 0.907 | 0.899 | 0.914 | 0.921 | 0.868 | 0.974 | 0.936 | 0.875 | 0.996 |
| IFI27_ISG15_OLAH | 0.906 | 0.899 | 0.914 | 0.916 | 0.859 | 0.973 | 0.934 | 0.871 | 0.996 |
| IFI27_OLAH_RETN | 0.905 | 0.897 | 0.913 | 0.916 | 0.857 | 0.974 | 0.940 | 0.876 | 1 |
| IFI27_SLC1A2_OLAH | 0.905 | 0.897 | 0.913 | 0.926 | 0.874 | 0.978 | 0.929 | 0.862 | 0.997 |
| IFI27_SIGLEC1_OLAH | 0.905 | 0.897 | 0.913 | 0.917 | 0.859 | 0.975 | 0.945 | 0.890 | 0.999 |
| IFI27_SIGLEC1_IL1R2 | 0.905 | 0.898 | 0.912 | 0.920 | 0.871 | 0.969 | 0.951 | 0.905 | 0.998 |
| IFI27_IFI44L_OLAH | 0.905 | 0.896 | 0.913 | 0.916 | 0.859 | 0.974 | 0.937 | 0.876 | 0.997 |
| IFI27_IFIT1_OLAH | 0.904 | 0.896 | 0.912 | 0.916 | 0.858 | 0.974 | 0.939 | 0.881 | 0.998 |
| IFI27_OAS1_OLAH | 0.903 | 0.896 | 0.911 | 0.916 | 0.858 | 0.974 | 0.938 | 0.879 | 0.997 |
| IFI27_HERC6_IL1R2 | 0.903 | 0.896 | 0.910 | 0.918 | 0.870 | 0.966 | 0.929 | 0.870 | 0.988 |
| RSAD2_IFI27_OLAH | 0.903 | 0.895 | 0.911 | 0.916 | 0.858 | 0.974 | 0.939 | 0.881 | 0.998 |
| IFI27_IL1R2_RETN | 0.902 | 0.895 | 0.910 | 0.921 | 0.871 | 0.971 | 0.924 | 0.852 | 0.995 |
| IFI27_IFI44L_IL1R2 | 0.901 | 0.894 | 0.908 | 0.913 | 0.863 | 0.963 | 0.932 | 0.872 | 0.991 |
| IFI27_IFIT1_IL1R2 | 0.901 | 0.893 | 0.908 | 0.915 | 0.864 | 0.966 | 0.940 | 0.887 | 0.994 |
| IFI27_ISG15_IL1R2 | 0.900 | 0.893 | 0.906 | 0.914 | 0.864 | 0.963 | 0.929 | 0.868 | 0.991 |
| RSAD2_IFI27_IL1R2 | 0.900 | 0.892 | 0.907 | 0.916 | 0.866 | 0.966 | 0.935 | 0.878 | 0.992 |
| IFI27_OAS1_IL1R2 | 0.899 | 0.892 | 0.906 | 0.914 | 0.864 | 0.965 | 0.932 | 0.873 | 0.990 |
| IFI27_IL1R2_MMP8 | 0.898 | 0.891 | 0.905 | 0.913 | 0.863 | 0.962 | 0.914 | 0.844 | 0.984 |
| IFI27_SIGLEC1_FAM20A | 0.896 | 0.887 | 0.905 | 0.905 | 0.837 | 0.974 | 0.950 | 0.874 | 1 |
| SIGLEC1_IL1R2_FAM20A | 0.896 | 0.888 | 0.905 | 0.909 | 0.846 | 0.973 | 0.963 | 0.912 | 1 |
| IFI27_SLC1A2_IL1R2 | 0.895 | 0.888 | 0.903 | 0.917 | 0.871 | 0.963 | 0.903 | 0.819 | 0.988 |
| IFI27_HERC6_RETN | 0.895 | 0.885 | 0.904 | 0.908 | 0.845 | 0.971 | 0.951 | 0.893 | 1 |
| IFI27_FAM20A_RETN | 0.894 | 0.884 | 0.904 | 0.906 | 0.844 | 0.968 | 0.923 | 0.832 | 1 |
| HERC6_IL1R2_FAM20A | 0.892 | 0.883 | 0.900 | 0.907 | 0.847 | 0.966 | 0.922 | 0.833 | 1 |
| SIGLEC1_SLC1A2_OLAH | 0.892 | 0.883 | 0.900 | 0.912 | 0.857 | 0.968 | 0.951 | 0.904 | 0.998 |
| IFI27_SIGLEC1_RETN | 0.891 | 0.882 | 0.901 | 0.904 | 0.840 | 0.969 | 0.962 | 0.912 | 1 |
| IFIT1_SIGLEC1_FAM20A | 0.890 | 0.881 | 0.899 | 0.897 | 0.826 | 0.968 | 0.939 | 0.873 | 1 |
| IFI27_IFI44L_RETN | 0.890 | 0.880 | 0.899 | 0.906 | 0.842 | 0.970 | 0.955 | 0.901 | 1 |
| SIGLEC1_HERC6_IL1R2 | 0.889 | 0.881 | 0.897 | 0.902 | 0.841 | 0.962 | 0.953 | 0.909 | 0.998 |
| SIGLEC1_OLAH_FAM20A | 0.889 | 0.879 | 0.898 | 0.897 | 0.825 | 0.969 | 0.962 | 0.918 | 1 |
| IFI27_HERC6_FAM20A | 0.887 | 0.878 | 0.897 | 0.902 | 0.839 | 0.965 | 0.934 | 0.851 | 1 |
| IFI27_ISG15_RETN | 0.887 | 0.878 | 0.897 | 0.903 | 0.841 | 0.966 | 0.947 | 0.883 | 1 |
| SIGLEC1_IL1R2_MMP8 | 0.887 | 0.879 | 0.895 | 0.901 | 0.842 | 0.960 | 0.962 | 0.923 | 1 |
| IFI27_IFIT1_RETN | 0.887 | 0.877 | 0.896 | 0.904 | 0.840 | 0.968 | 0.952 | 0.892 | 1 |
| IFI44L_SLC1A2_OLAH | 0.887 | 0.878 | 0.895 | 0.905 | 0.848 | 0.963 | 0.930 | 0.873 | 0.988 |
| IFI44L_SIGLEC1_IL1R2 | 0.886 | 0.878 | 0.894 | 0.898 | 0.836 | 0.960 | 0.958 | 0.916 | 0.999 |
| SIGLEC1_ISG15_IL1R2 | 0.886 | 0.878 | 0.894 | 0.899 | 0.837 | 0.961 | 0.962 | 0.923 | 1 |
| SIGLEC1_SLC1A2_IL1R2 | 0.886 | 0.878 | 0.894 | 0.908 | 0.853 | 0.964 | 0.964 | 0.927 | 1 |
| SIGLEC1_IL1R2_OLAH | 0.885 | 0.877 | 0.894 | 0.901 | 0.840 | 0.963 | 0.960 | 0.918 | 1 |
| SIGLEC1_SLC1A2_FAM20A | 0.885 | 0.876 | 0.894 | 0.902 | 0.839 | 0.966 | 0.949 | 0.877 | 1 |
| RSAD2_SIGLEC1_IL1R2 | 0.885 | 0.877 | 0.893 | 0.897 | 0.833 | 0.961 | 0.961 | 0.922 | 1.000 |
| IFIT1_SIGLEC1_IL1R2 | 0.885 | 0.876 | 0.893 | 0.898 | 0.836 | 0.960 | 0.958 | 0.916 | 0.999 |
| OAS1_SIGLEC1_IL1R2 | 0.884 | 0.876 | 0.893 | 0.898 | 0.835 | 0.961 | 0.959 | 0.918 | 0.999 |
| HERC6_OLAH_FAM20A | 0.884 | 0.875 | 0.894 | 0.898 | 0.833 | 0.963 | 0.942 | 0.875 | 1 |
| SIGLEC1_FAM20A_RETN | 0.884 | 0.875 | 0.894 | 0.893 | 0.821 | 0.966 | 0.954 | 0.895 | 1 |
| OAS1_IL1R2_FAM20A | 0.884 | 0.876 | 0.893 | 0.898 | 0.831 | 0.964 | 0.935 | 0.856 | 1 |
| SIGLEC1_HERC6_FAM20A | 0.884 | 0.874 | 0.894 | 0.893 | 0.821 | 0.965 | 0.952 | 0.879 | 1 |
| IFI44L_SIGLEC1_FAM20A | 0.884 | 0.874 | 0.893 | 0.893 | 0.822 | 0.965 | 0.958 | 0.898 | 1 |
| IFI44L_IL1R2_FAM20A | 0.884 | 0.875 | 0.892 | 0.896 | 0.832 | 0.960 | 0.943 | 0.874 | 1 |
| IFI27_OAS1_RETN | 0.884 | 0.874 | 0.893 | 0.903 | 0.839 | 0.966 | 0.948 | 0.889 | 1 |
| RSAD2_IFI27_RETN | 0.883 | 0.874 | 0.893 | 0.901 | 0.837 | 0.966 | 0.952 | 0.894 | 1 |
| SIGLEC1_FAM20A_MMP8 | 0.883 | 0.873 | 0.893 | 0.890 | 0.817 | 0.964 | 0.959 | 0.897 | 1 |
| SIGLEC1_IL1R2_RETN | 0.883 | 0.874 | 0.891 | 0.896 | 0.832 | 0.961 | 0.960 | 0.918 | 1 |
| ISG15_IL1R2_FAM20A | 0.882 | 0.874 | 0.891 | 0.900 | 0.838 | 0.961 | 0.926 | 0.844 | 1 |
| SIGLEC1_ISG15_FAM20A | 0.882 | 0.873 | 0.892 | 0.890 | 0.816 | 0.963 | 0.955 | 0.893 | 1 |
| RSAD2_SIGLEC1_FAM20A | 0.882 | 0.873 | 0.892 | 0.895 | 0.824 | 0.965 | 0.952 | 0.890 | 1 |
| IFIT1_HERC6_IL1R2 | 0.882 | 0.874 | 0.890 | 0.895 | 0.833 | 0.958 | 0.942 | 0.891 | 0.994 |
| IFI27_IFI44L_FAM20A | 0.882 | 0.872 | 0.891 | 0.895 | 0.825 | 0.964 | 0.939 | 0.860 | 1 |
| ISG15_HERC6_IL1R2 | 0.882 | 0.874 | 0.890 | 0.895 | 0.836 | 0.954 | 0.941 | 0.888 | 0.995 |
| OAS1_SIGLEC1_FAM20A | 0.882 | 0.872 | 0.891 | 0.892 | 0.820 | 0.964 | 0.955 | 0.895 | 1 |
| IFIT1_SLC1A2_OLAH | 0.881 | 0.872 | 0.890 | 0.903 | 0.844 | 0.962 | 0.943 | 0.893 | 0.994 |
| IFIT1_IL1R2_FAM20A | 0.881 | 0.872 | 0.890 | 0.899 | 0.835 | 0.964 | 0.950 | 0.884 | 1 |
| IFI27_RETN_MMP8 | 0.881 | 0.871 | 0.891 | 0.899 | 0.836 | 0.962 | 0.923 | 0.842 | 1 |
| IFI44L_HERC6_IL1R2 | 0.881 | 0.873 | 0.889 | 0.897 | 0.838 | 0.956 | 0.941 | 0.889 | 0.994 |
| RSAD2_IL1R2_FAM20A | 0.880 | 0.872 | 0.889 | 0.894 | 0.827 | 0.961 | 0.942 | 0.867 | 1 |
| IFI27_SLC1A2_RETN | 0.880 | 0.870 | 0.889 | 0.893 | 0.827 | 0.960 | 0.925 | 0.843 | 1 |
| OAS1_SLC1A2_OLAH | 0.879 | 0.870 | 0.888 | 0.906 | 0.849 | 0.963 | 0.930 | 0.869 | 0.992 |
| HERC6_IL1R2_OLAH | 0.879 | 0.870 | 0.888 | 0.898 | 0.839 | 0.957 | 0.935 | 0.878 | 0.991 |
| SIGLEC1_HERC6_OLAH | 0.879 | 0.870 | 0.888 | 0.893 | 0.825 | 0.961 | 0.957 | 0.914 | 0.999 |
| OAS1_HERC6_IL1R2 | 0.879 | 0.870 | 0.887 | 0.893 | 0.831 | 0.955 | 0.932 | 0.873 | 0.990 |
| HERC6_SLC1A2_OLAH | 0.879 | 0.870 | 0.888 | 0.902 | 0.844 | 0.960 | 0.937 | 0.880 | 0.993 |
| RSAD2_HERC6_IL1R2 | 0.878 | 0.870 | 0.887 | 0.894 | 0.832 | 0.955 | 0.943 | 0.892 | 0.995 |
| RSAD2_SLC1A2_OLAH | 0.878 | 0.869 | 0.887 | 0.901 | 0.843 | 0.960 | 0.935 | 0.878 | 0.991 |
| HERC6_IL1R2_RETN | 0.878 | 0.869 | 0.887 | 0.893 | 0.829 | 0.958 | 0.929 | 0.869 | 0.990 |
| OAS1_OLAH_FAM20A | 0.878 | 0.868 | 0.887 | 0.891 | 0.819 | 0.963 | 0.949 | 0.886 | 1 |
| IFIT1_SIGLEC1_OLAH | 0.878 | 0.869 | 0.886 | 0.893 | 0.825 | 0.960 | 0.943 | 0.890 | 0.997 |
| IFI27_ISG15_FAM20A | 0.878 | 0.867 | 0.888 | 0.886 | 0.813 | 0.960 | 0.930 | 0.850 | 1 |
| IFIT1_SLC1A2_IL1R2 | 0.878 | 0.869 | 0.886 | 0.900 | 0.841 | 0.959 | 0.947 | 0.896 | 0.997 |
| IFI44L_SIGLEC1_OLAH | 0.878 | 0.869 | 0.886 | 0.891 | 0.822 | 0.960 | 0.947 | 0.896 | 0.998 |
| IFI27_HERC6_MMP8 | 0.877 | 0.868 | 0.887 | 0.891 | 0.826 | 0.956 | 0.905 | 0.829 | 0.982 |
| HERC6_IL1R2_MMP8 | 0.877 | 0.869 | 0.886 | 0.896 | 0.837 | 0.955 | 0.926 | 0.865 | 0.988 |
| SIGLEC1_OLAH_MMP8 | 0.877 | 0.868 | 0.887 | 0.890 | 0.822 | 0.958 | 0.954 | 0.910 | 0.999 |
| IFI44L_SLC1A2_IL1R2 | 0.877 | 0.869 | 0.885 | 0.896 | 0.838 | 0.953 | 0.934 | 0.871 | 0.997 |
| OAS1_SIGLEC1_OLAH | 0.876 | 0.867 | 0.886 | 0.889 | 0.819 | 0.959 | 0.948 | 0.898 | 0.998 |
| SIGLEC1_ISG15_OLAH | 0.876 | 0.867 | 0.885 | 0.890 | 0.821 | 0.959 | 0.949 | 0.900 | 0.998 |
| SIGLEC1_OLAH_RETN | 0.876 | 0.866 | 0.885 | 0.890 | 0.819 | 0.961 | 0.945 | 0.890 | 0.999 |
| HERC6_SLC1A2_IL1R2 | 0.876 | 0.867 | 0.885 | 0.901 | 0.847 | 0.956 | 0.914 | 0.835 | 0.993 |
| IFIT1_IL1R2_MMP8 | 0.875 | 0.867 | 0.884 | 0.892 | 0.829 | 0.956 | 0.959 | 0.918 | 0.999 |
| OAS1_IFIT1_IL1R2 | 0.875 | 0.866 | 0.884 | 0.888 | 0.821 | 0.956 | 0.945 | 0.895 | 0.994 |
| ISG15_SLC1A2_OLAH | 0.875 | 0.866 | 0.883 | 0.895 | 0.835 | 0.954 | 0.922 | 0.854 | 0.989 |
| IFIT1_ISG15_IL1R2 | 0.875 | 0.866 | 0.883 | 0.894 | 0.830 | 0.958 | 0.958 | 0.916 | 0.999 |
| IFI27_FAM20A_MMP8 | 0.875 | 0.865 | 0.885 | 0.887 | 0.818 | 0.957 | 0.907 | 0.814 | 0.999 |
| IFI27_IFIT1_FAM20A | 0.875 | 0.864 | 0.885 | 0.888 | 0.817 | 0.959 | 0.941 | 0.862 | 1 |
| RSAD2_SIGLEC1_OLAH | 0.875 | 0.865 | 0.884 | 0.890 | 0.821 | 0.959 | 0.950 | 0.902 | 0.998 |
| HERC6_FAM20A_RETN | 0.875 | 0.864 | 0.885 | 0.887 | 0.816 | 0.958 | 0.939 | 0.861 | 1 |
| IFI44L_OLAH_FAM20A | 0.874 | 0.865 | 0.884 | 0.889 | 0.818 | 0.960 | 0.957 | 0.909 | 1 |
| HERC6_OLAH_RETN | 0.874 | 0.864 | 0.884 | 0.889 | 0.818 | 0.959 | 0.942 | 0.891 | 0.993 |
| OAS1_IL1R2_OLAH | 0.873 | 0.864 | 0.882 | 0.894 | 0.828 | 0.960 | 0.938 | 0.879 | 0.997 |
| IFIT1_IFI44L_IL1R2 | 0.873 | 0.864 | 0.882 | 0.890 | 0.824 | 0.956 | 0.953 | 0.909 | 0.997 |
| IFI27_SIGLEC1_MMP8 | 0.873 | 0.863 | 0.882 | 0.883 | 0.813 | 0.954 | 0.916 | 0.844 | 0.988 |
| IFI44L_ISG15_IL1R2 | 0.872 | 0.864 | 0.880 | 0.889 | 0.829 | 0.949 | 0.954 | 0.910 | 0.999 |
| IFI27_OAS1_FAM20A | 0.872 | 0.862 | 0.882 | 0.883 | 0.811 | 0.956 | 0.942 | 0.864 | 1 |
| ISG15_SLC1A2_IL1R2 | 0.872 | 0.864 | 0.880 | 0.894 | 0.836 | 0.952 | 0.917 | 0.838 | 0.997 |
| IFI27_SLC1A2_FAM20A | 0.872 | 0.861 | 0.882 | 0.884 | 0.812 | 0.956 | 0.915 | 0.827 | 1 |
| HERC6_OLAH_MMP8 | 0.872 | 0.862 | 0.881 | 0.887 | 0.820 | 0.955 | 0.941 | 0.889 | 0.994 |
| IFIT1_IL1R2_OLAH | 0.872 | 0.863 | 0.881 | 0.891 | 0.825 | 0.957 | 0.954 | 0.910 | 0.998 |
| IFI44L_HERC6_OLAH | 0.871 | 0.862 | 0.881 | 0.887 | 0.820 | 0.954 | 0.941 | 0.889 | 0.993 |
| RSAD2_SLC1A2_IL1R2 | 0.871 | 0.863 | 0.879 | 0.895 | 0.837 | 0.954 | 0.937 | 0.874 | 1 |
| SIGLEC1_HERC6_RETN | 0.871 | 0.861 | 0.881 | 0.878 | 0.799 | 0.958 | 0.959 | 0.916 | 1 |
| IFI44L_IL1R2_OLAH | 0.871 | 0.862 | 0.880 | 0.889 | 0.827 | 0.952 | 0.942 | 0.889 | 0.995 |
| RSAD2_IFIT1_IL1R2 | 0.871 | 0.862 | 0.880 | 0.888 | 0.820 | 0.956 | 0.953 | 0.909 | 0.997 |
| ISG15_IL1R2_OLAH | 0.871 | 0.862 | 0.879 | 0.889 | 0.827 | 0.950 | 0.938 | 0.876 | 1.000 |
| IFIT1_HERC6_OLAH | 0.871 | 0.861 | 0.880 | 0.886 | 0.819 | 0.953 | 0.940 | 0.888 | 0.993 |
| IFIT1_IL1R2_RETN | 0.870 | 0.861 | 0.880 | 0.888 | 0.819 | 0.957 | 0.954 | 0.910 | 0.998 |
| OAS1_HERC6_OLAH | 0.870 | 0.861 | 0.880 | 0.886 | 0.819 | 0.953 | 0.941 | 0.889 | 0.993 |
| ISG15_HERC6_OLAH | 0.870 | 0.861 | 0.880 | 0.885 | 0.818 | 0.952 | 0.950 | 0.904 | 0.996 |
| RSAD2_IFI27_FAM20A | 0.870 | 0.860 | 0.880 | 0.882 | 0.810 | 0.955 | 0.945 | 0.866 | 1 |
| OAS1_SLC1A2_IL1R2 | 0.870 | 0.861 | 0.878 | 0.896 | 0.837 | 0.954 | 0.917 | 0.844 | 0.991 |
| OAS1_IFI44L_IL1R2 | 0.870 | 0.861 | 0.879 | 0.886 | 0.821 | 0.952 | 0.935 | 0.878 | 0.991 |
| RSAD2_OLAH_FAM20A | 0.870 | 0.860 | 0.879 | 0.884 | 0.811 | 0.958 | 0.959 | 0.905 | 1 |
| SIGLEC1_SLC1A2_RETN | 0.870 | 0.861 | 0.879 | 0.893 | 0.829 | 0.957 | 0.950 | 0.902 | 0.998 |
| RSAD2_HERC6_OLAH | 0.870 | 0.860 | 0.879 | 0.886 | 0.820 | 0.952 | 0.939 | 0.886 | 0.992 |
| RSAD2_IL1R2_OLAH | 0.870 | 0.861 | 0.879 | 0.890 | 0.824 | 0.955 | 0.949 | 0.900 | 0.998 |
| IFI27_SIGLEC1_SLC1A2 | 0.869 | 0.860 | 0.878 | 0.889 | 0.826 | 0.952 | 0.964 | 0.922 | 1 |
| RSAD2_OAS1_IL1R2 | 0.869 | 0.860 | 0.878 | 0.886 | 0.820 | 0.952 | 0.934 | 0.877 | 0.991 |
| IFI44L_IL1R2_MMP8 | 0.868 | 0.860 | 0.876 | 0.883 | 0.821 | 0.945 | 0.949 | 0.900 | 0.997 |
| IFI27_IFI44L_SIGLEC1 | 0.868 | 0.859 | 0.877 | 0.886 | 0.821 | 0.950 | 0.945 | 0.893 | 0.996 |
| IFI27_SLC1A2_MMP8 | 0.868 | 0.858 | 0.877 | 0.886 | 0.821 | 0.951 | 0.892 | 0.805 | 0.980 |
| OAS1_IL1R2_MMP8 | 0.868 | 0.859 | 0.876 | 0.886 | 0.821 | 0.952 | 0.928 | 0.867 | 0.990 |
| RSAD2_IFI27_SIGLEC1 | 0.868 | 0.858 | 0.877 | 0.884 | 0.822 | 0.947 | 0.940 | 0.885 | 0.996 |
| OAS1_ISG15_IL1R2 | 0.868 | 0.859 | 0.876 | 0.888 | 0.823 | 0.952 | 0.932 | 0.871 | 0.992 |
| ISG15_IL1R2_RETN | 0.867 | 0.859 | 0.876 | 0.889 | 0.827 | 0.952 | 0.935 | 0.869 | 1 |
| IFI27_IFI44L_MMP8 | 0.867 | 0.857 | 0.877 | 0.878 | 0.809 | 0.948 | 0.902 | 0.823 | 0.981 |
| ISG15_OLAH_FAM20A | 0.867 | 0.857 | 0.877 | 0.887 | 0.820 | 0.954 | 0.945 | 0.892 | 0.997 |
| RSAD2_IFI44L_IL1R2 | 0.867 | 0.858 | 0.876 | 0.882 | 0.815 | 0.949 | 0.945 | 0.895 | 0.994 |
| IFI27_OAS1_SIGLEC1 | 0.867 | 0.857 | 0.876 | 0.882 | 0.818 | 0.946 | 0.945 | 0.892 | 0.997 |
| IFIT1_OLAH_FAM20A | 0.867 | 0.857 | 0.877 | 0.881 | 0.807 | 0.954 | 0.958 | 0.909 | 1 |
| IFIT1_SIGLEC1_RETN | 0.866 | 0.857 | 0.876 | 0.878 | 0.804 | 0.953 | 0.942 | 0.889 | 0.996 |
| IFI27_IFIT1_SIGLEC1 | 0.866 | 0.857 | 0.876 | 0.885 | 0.823 | 0.947 | 0.941 | 0.886 | 0.996 |
| RSAD2_ISG15_IL1R2 | 0.866 | 0.858 | 0.875 | 0.883 | 0.818 | 0.949 | 0.953 | 0.908 | 0.998 |
| ISG15_IL1R2_MMP8 | 0.866 | 0.858 | 0.874 | 0.884 | 0.824 | 0.944 | 0.933 | 0.865 | 1.000 |
| OAS1_IL1R2_RETN | 0.866 | 0.857 | 0.876 | 0.885 | 0.817 | 0.954 | 0.932 | 0.871 | 0.992 |
| IFI44L_IL1R2_RETN | 0.866 | 0.857 | 0.875 | 0.885 | 0.818 | 0.951 | 0.946 | 0.897 | 0.995 |
| RSAD2_OAS1_OLAH | 0.866 | 0.856 | 0.875 | 0.880 | 0.808 | 0.952 | 0.943 | 0.889 | 0.998 |
| RSAD2_IL1R2_MMP8 | 0.866 | 0.857 | 0.874 | 0.883 | 0.818 | 0.949 | 0.948 | 0.899 | 0.996 |
| SIGLEC1_SLC1A2_MMP8 | 0.866 | 0.857 | 0.874 | 0.884 | 0.819 | 0.949 | 0.937 | 0.882 | 0.992 |
| OAS1_OLAH_RETN | 0.866 | 0.856 | 0.875 | 0.884 | 0.811 | 0.958 | 0.945 | 0.891 | 0.998 |
| ISG15_HERC6_RETN | 0.865 | 0.855 | 0.876 | 0.878 | 0.801 | 0.955 | 0.954 | 0.908 | 1 |
| RSAD2_IL1R2_RETN | 0.865 | 0.856 | 0.874 | 0.883 | 0.813 | 0.953 | 0.946 | 0.896 | 0.995 |
| OAS1_IFIT1_OLAH | 0.865 | 0.856 | 0.875 | 0.880 | 0.806 | 0.953 | 0.943 | 0.891 | 0.996 |
| IFI27_ISG15_MMP8 | 0.865 | 0.856 | 0.874 | 0.878 | 0.808 | 0.948 | 0.899 | 0.818 | 0.979 |
| IFI27_IFIT1_MMP8 | 0.865 | 0.855 | 0.875 | 0.876 | 0.805 | 0.946 | 0.913 | 0.839 | 0.987 |
| SIGLEC1_ISG15_RETN | 0.864 | 0.854 | 0.874 | 0.875 | 0.798 | 0.952 | 0.953 | 0.907 | 0.999 |
| OAS1_OLAH_MMP8 | 0.864 | 0.855 | 0.874 | 0.879 | 0.806 | 0.951 | 0.949 | 0.899 | 0.999 |
| IFI44L_SIGLEC1_RETN | 0.864 | 0.854 | 0.874 | 0.876 | 0.799 | 0.953 | 0.948 | 0.899 | 0.997 |
| HERC6_SLC1A2_FAM20A | 0.864 | 0.854 | 0.874 | 0.886 | 0.824 | 0.949 | 0.928 | 0.843 | 1 |
| ISG15_HERC6_FAM20A | 0.864 | 0.854 | 0.874 | 0.875 | 0.803 | 0.947 | 0.930 | 0.845 | 1 |
| OAS1_SIGLEC1_RETN | 0.864 | 0.853 | 0.874 | 0.876 | 0.799 | 0.953 | 0.946 | 0.894 | 0.997 |
| HERC6_FAM20A_MMP8 | 0.863 | 0.853 | 0.874 | 0.878 | 0.808 | 0.949 | 0.927 | 0.845 | 1 |
| RSAD2_SIGLEC1_RETN | 0.863 | 0.853 | 0.874 | 0.876 | 0.799 | 0.953 | 0.945 | 0.893 | 0.996 |
| OAS1_IFI44L_OLAH | 0.863 | 0.853 | 0.872 | 0.882 | 0.810 | 0.953 | 0.941 | 0.887 | 0.996 |
| SLC1A2_OLAH_FAM20A | 0.863 | 0.853 | 0.872 | 0.884 | 0.822 | 0.945 | 0.859 | 0.754 | 0.963 |
| IFI27_SIGLEC1_ISG15 | 0.863 | 0.853 | 0.873 | 0.880 | 0.815 | 0.945 | 0.950 | 0.899 | 1 |
| IFI27_OAS1_MMP8 | 0.862 | 0.852 | 0.872 | 0.875 | 0.804 | 0.946 | 0.901 | 0.822 | 0.980 |
| OAS1_ISG15_OLAH | 0.862 | 0.852 | 0.871 | 0.880 | 0.808 | 0.952 | 0.943 | 0.890 | 0.997 |
| RSAD2_IFI27_MMP8 | 0.862 | 0.852 | 0.871 | 0.877 | 0.806 | 0.947 | 0.908 | 0.830 | 0.985 |
| IFI44L_SLC1A2_FAM20A | 0.861 | 0.852 | 0.871 | 0.879 | 0.810 | 0.948 | 0.925 | 0.843 | 1 |
| IFI44L_OLAH_MMP8 | 0.861 | 0.852 | 0.871 | 0.872 | 0.800 | 0.944 | 0.937 | 0.881 | 0.993 |
| SIGLEC1_RETN_MMP8 | 0.861 | 0.851 | 0.872 | 0.873 | 0.796 | 0.951 | 0.953 | 0.906 | 1 |
| HERC6_SLC1A2_RETN | 0.861 | 0.851 | 0.871 | 0.885 | 0.818 | 0.953 | 0.933 | 0.866 | 0.999 |
| IFIT1_SIGLEC1_SLC1A2 | 0.861 | 0.852 | 0.869 | 0.878 | 0.813 | 0.943 | 0.950 | 0.904 | 0.996 |
| IFI44L_ISG15_OLAH | 0.861 | 0.851 | 0.870 | 0.876 | 0.805 | 0.946 | 0.942 | 0.891 | 0.994 |
| RSAD2_OLAH_RETN | 0.861 | 0.851 | 0.871 | 0.877 | 0.800 | 0.953 | 0.958 | 0.914 | 1 |
| IFI27_SIGLEC1_HERC6 | 0.860 | 0.850 | 0.871 | 0.878 | 0.812 | 0.944 | 0.947 | 0.897 | 0.997 |
| SIGLEC1_HERC6_SLC1A2 | 0.860 | 0.852 | 0.869 | 0.877 | 0.810 | 0.944 | 0.954 | 0.908 | 1 |
| OAS1_SIGLEC1_SLC1A2 | 0.860 | 0.851 | 0.869 | 0.876 | 0.811 | 0.942 | 0.951 | 0.906 | 0.997 |
| IFI44L_HERC6_RETN | 0.860 | 0.850 | 0.870 | 0.871 | 0.792 | 0.950 | 0.937 | 0.879 | 0.995 |
| RSAD2_OLAH_MMP8 | 0.860 | 0.850 | 0.869 | 0.874 | 0.801 | 0.947 | 0.950 | 0.902 | 0.998 |
| IFIT1_HERC6_RETN | 0.860 | 0.849 | 0.870 | 0.873 | 0.796 | 0.950 | 0.927 | 0.865 | 0.989 |
| IFI44L_OLAH_RETN | 0.860 | 0.849 | 0.870 | 0.877 | 0.801 | 0.952 | 0.939 | 0.883 | 0.995 |
| IFIT1_HERC6_FAM20A | 0.860 | 0.849 | 0.870 | 0.873 | 0.801 | 0.945 | 0.928 | 0.843 | 1 |
| RSAD2_SIGLEC1_SLC1A2 | 0.859 | 0.851 | 0.868 | 0.877 | 0.811 | 0.943 | 0.948 | 0.900 | 0.996 |
| SLC1A2_IL1R2_OLAH | 0.859 | 0.850 | 0.868 | 0.877 | 0.815 | 0.939 | 0.842 | 0.733 | 0.952 |
| RSAD2_IFI27_HERC6 | 0.859 | 0.850 | 0.869 | 0.880 | 0.820 | 0.940 | 0.886 | 0.807 | 0.965 |
| SIGLEC1_HERC6_MMP8 | 0.859 | 0.849 | 0.869 | 0.871 | 0.795 | 0.947 | 0.914 | 0.848 | 0.981 |
| IFIT1_ISG15_OLAH | 0.859 | 0.850 | 0.869 | 0.875 | 0.803 | 0.948 | 0.957 | 0.913 | 1.000 |
| IFIT1_OLAH_RETN | 0.859 | 0.849 | 0.869 | 0.878 | 0.803 | 0.953 | 0.953 | 0.908 | 0.998 |
| IFIT1_OLAH_MMP8 | 0.859 | 0.849 | 0.869 | 0.870 | 0.796 | 0.945 | 0.953 | 0.909 | 0.998 |
| IFI44L_SIGLEC1_SLC1A2 | 0.859 | 0.851 | 0.867 | 0.880 | 0.814 | 0.945 | 0.955 | 0.913 | 0.998 |
| IFI44L_HERC6_FAM20A | 0.859 | 0.849 | 0.869 | 0.870 | 0.796 | 0.945 | 0.934 | 0.850 | 1 |
| ISG15_OLAH_RETN | 0.859 | 0.849 | 0.868 | 0.879 | 0.810 | 0.948 | 0.948 | 0.894 | 1 |
| IFI27_HERC6_SLC1A2 | 0.859 | 0.849 | 0.869 | 0.880 | 0.817 | 0.943 | 0.921 | 0.847 | 0.994 |
| IFI27_OAS1_HERC6 | 0.859 | 0.849 | 0.869 | 0.880 | 0.819 | 0.940 | 0.901 | 0.829 | 0.973 |
| IFIT1_IFI44L_OLAH | 0.859 | 0.849 | 0.868 | 0.876 | 0.803 | 0.948 | 0.941 | 0.888 | 0.994 |
| OAS1_FAM20A_RETN | 0.858 | 0.848 | 0.869 | 0.880 | 0.806 | 0.954 | 0.934 | 0.852 | 1 |
| RSAD2_IFIT1_OLAH | 0.858 | 0.849 | 0.868 | 0.875 | 0.802 | 0.948 | 0.951 | 0.904 | 0.998 |
| OAS1_HERC6_RETN | 0.858 | 0.848 | 0.868 | 0.870 | 0.793 | 0.948 | 0.925 | 0.861 | 0.989 |
| IFI27_IFI44L_SLC1A2 | 0.858 | 0.849 | 0.868 | 0.877 | 0.809 | 0.945 | 0.927 | 0.863 | 0.991 |
| SIGLEC1_ISG15_SLC1A2 | 0.858 | 0.850 | 0.866 | 0.878 | 0.811 | 0.944 | 0.954 | 0.910 | 0.998 |
| IFI27_ISG15_HERC6 | 0.858 | 0.847 | 0.869 | 0.878 | 0.815 | 0.941 | 0.911 | 0.843 | 0.979 |
| IFI27_IFI44L_HERC6 | 0.858 | 0.848 | 0.868 | 0.876 | 0.813 | 0.939 | 0.907 | 0.837 | 0.976 |
| ISG15_OLAH_MMP8 | 0.858 | 0.849 | 0.867 | 0.875 | 0.807 | 0.942 | 0.943 | 0.892 | 0.995 |
| SLC1A2_OLAH_MMP8 | 0.858 | 0.849 | 0.867 | 0.879 | 0.817 | 0.941 | 0.855 | 0.754 | 0.957 |
| RSAD2_ISG15_OLAH | 0.858 | 0.848 | 0.867 | 0.877 | 0.805 | 0.948 | 0.952 | 0.906 | 0.998 |
| SLC1A2_OLAH_RETN | 0.858 | 0.848 | 0.867 | 0.878 | 0.815 | 0.941 | 0.842 | 0.733 | 0.952 |
| RSAD2_HERC6_FAM20A | 0.858 | 0.848 | 0.868 | 0.874 | 0.804 | 0.945 | 0.924 | 0.837 | 1 |
| RSAD2_HERC6_RETN | 0.858 | 0.848 | 0.868 | 0.871 | 0.794 | 0.948 | 0.922 | 0.857 | 0.987 |
| IFI27_ISG15_SLC1A2 | 0.857 | 0.848 | 0.867 | 0.875 | 0.803 | 0.946 | 0.918 | 0.845 | 0.992 |
| RSAD2_IFI44L_OLAH | 0.857 | 0.848 | 0.867 | 0.876 | 0.803 | 0.948 | 0.946 | 0.896 | 0.996 |
| IFI27_IFIT1_SLC1A2 | 0.857 | 0.847 | 0.867 | 0.877 | 0.809 | 0.945 | 0.949 | 0.893 | 1 |
| HERC6_RETN_MMP8 | 0.857 | 0.846 | 0.867 | 0.870 | 0.792 | 0.948 | 0.916 | 0.850 | 0.983 |
| OAS1_HERC6_FAM20A | 0.857 | 0.846 | 0.867 | 0.870 | 0.797 | 0.944 | 0.933 | 0.848 | 1 |
| RSAD2_SIGLEC1_HERC6 | 0.856 | 0.846 | 0.865 | 0.869 | 0.803 | 0.936 | 0.911 | 0.843 | 0.979 |
| IFIT1_SIGLEC1_HERC6 | 0.855 | 0.846 | 0.864 | 0.867 | 0.798 | 0.937 | 0.912 | 0.845 | 0.979 |
| IFIT1_SIGLEC1_MMP8 | 0.855 | 0.845 | 0.865 | 0.867 | 0.793 | 0.942 | 0.913 | 0.846 | 0.980 |
| HERC6_SLC1A2_MMP8 | 0.855 | 0.845 | 0.864 | 0.875 | 0.807 | 0.943 | 0.892 | 0.809 | 0.976 |
| OAS1_SIGLEC1_HERC6 | 0.854 | 0.845 | 0.864 | 0.866 | 0.798 | 0.935 | 0.941 | 0.890 | 0.993 |
| IFI27_IFIT1_HERC6 | 0.854 | 0.843 | 0.864 | 0.877 | 0.813 | 0.940 | 0.917 | 0.852 | 0.982 |
| IFI44L_SIGLEC1_MMP8 | 0.853 | 0.843 | 0.863 | 0.863 | 0.787 | 0.940 | 0.924 | 0.863 | 0.985 |
| IFI44L_FAM20A_RETN | 0.853 | 0.842 | 0.863 | 0.867 | 0.790 | 0.944 | 0.937 | 0.867 | 1 |
| IFI27_OAS1_SLC1A2 | 0.853 | 0.843 | 0.863 | 0.871 | 0.800 | 0.942 | 0.920 | 0.844 | 0.995 |
| RSAD2_IFI27_SLC1A2 | 0.853 | 0.843 | 0.863 | 0.871 | 0.800 | 0.942 | 0.921 | 0.846 | 0.996 |
| IFI44L_SLC1A2_RETN | 0.853 | 0.842 | 0.863 | 0.876 | 0.805 | 0.946 | 0.925 | 0.863 | 0.987 |
| IFIT1_SLC1A2_FAM20A | 0.852 | 0.842 | 0.863 | 0.869 | 0.797 | 0.942 | 0.933 | 0.851 | 1 |
| OAS1_SIGLEC1_MMP8 | 0.852 | 0.842 | 0.862 | 0.864 | 0.788 | 0.940 | 0.923 | 0.861 | 0.985 |
| IFI44L_ISG15_FAM20A | 0.852 | 0.842 | 0.861 | 0.860 | 0.779 | 0.941 | 0.940 | 0.864 | 1 |
| IFI44L_SIGLEC1_HERC6 | 0.852 | 0.842 | 0.861 | 0.862 | 0.790 | 0.935 | 0.938 | 0.885 | 0.991 |
| SIGLEC1_ISG15_MMP8 | 0.851 | 0.841 | 0.861 | 0.865 | 0.788 | 0.941 | 0.926 | 0.866 | 0.986 |
| RSAD2_SIGLEC1_MMP8 | 0.851 | 0.841 | 0.861 | 0.864 | 0.788 | 0.940 | 0.921 | 0.858 | 0.984 |

Performance obtained for the identification of the viral or bacterial nature of the infection on the basis of the four-gene combinations:

TABLE 8

| | AUC | | | AUC | CI 95% | | AUC | CI 95% | |
|---|---|---|---|---|---|---|---|---|---|
| Model | model | CI 95% | | Train | Train | | Test | Test | |
| IFI27_SIGLEC1_IL1R2_FAM20A | 0.909 | 0.901 | 0.917 | 0.926 | 0.871 | 0.981 | 0.949 | 0.881 | 1 |
| IFI27_IL1R2_OLAH_FAM20A | 0.909 | 0.901 | 0.917 | 0.926 | 0.870 | 0.981 | 0.926 | 0.852 | 1.000 |
| IFI27_HERC6_IL1R2_FAM20A | 0.909 | 0.901 | 0.917 | 0.927 | 0.877 | 0.978 | 0.927 | 0.845 | 1 |
| IFI27_OLAH_FAM20A_MMP8 | 0.909 | 0.900 | 0.917 | 0.922 | 0.864 | 0.980 | 0.929 | 0.858 | 1 |
| IFI27_HERC6_OLAH_FAM20A | 0.909 | 0.901 | 0.917 | 0.922 | 0.862 | 0.982 | 0.942 | 0.879 | 1 |
| IFI27_ISG15_OLAH_FAM20A | 0.908 | 0.900 | 0.916 | 0.922 | 0.863 | 0.980 | 0.925 | 0.851 | 0.999 |
| IFI27_IFI44L_IL1R2_FAM20A | 0.907 | 0.899 | 0.915 | 0.924 | 0.870 | 0.978 | 0.927 | 0.845 | 1 |
| IFI27_SIGLEC1_OLAH_FAM20A | 0.906 | 0.897 | 0.915 | 0.918 | 0.855 | 0.981 | 0.952 | 0.893 | 1 |
| IFI27_OLAH_FAM20A_RETN | 0.906 | 0.897 | 0.914 | 0.921 | 0.862 | 0.981 | 0.934 | 0.863 | 1 |
| IFI27_IFI44L_OLAH_FAM20A | 0.906 | 0.897 | 0.914 | 0.920 | 0.858 | 0.981 | 0.938 | 0.872 | 1 |
| IFI27_IFIT1_OLAH_FAM20A | 0.905 | 0.897 | 0.913 | 0.919 | 0.858 | 0.981 | 0.937 | 0.869 | 1 |
| RSAD2_IFI27_OLAH_FAM20A | 0.905 | 0.896 | 0.913 | 0.920 | 0.859 | 0.981 | 0.937 | 0.869 | 1 |
| IFI27_IFIT1_IL1R2_FAM20A | 0.905 | 0.896 | 0.913 | 0.923 | 0.869 | 0.977 | 0.929 | 0.850 | 1 |
| IFI27_OAS1_OLAH_FAM20A | 0.904 | 0.896 | 0.913 | 0.920 | 0.860 | 0.981 | 0.937 | 0.870 | 1 |
| IFI27_IL1R2_FAM20A_MMP8 | 0.904 | 0.896 | 0.912 | 0.921 | 0.870 | 0.973 | 0.908 | 0.815 | 1 |
| RSAD2_IFI27_HERC6_OLAH | 0.904 | 0.896 | 0.912 | 0.923 | 0.871 | 0.976 | 0.937 | 0.879 | 0.995 |
| IFI27_IFI44L_HERC6_OLAH | 0.904 | 0.896 | 0.912 | 0.924 | 0.872 | 0.977 | 0.937 | 0.880 | 0.994 |
| IFI27_OAS1_HERC6_OLAH | 0.904 | 0.896 | 0.912 | 0.923 | 0.871 | 0.975 | 0.935 | 0.876 | 0.994 |
| IFI27_OAS1_IL1R2_FAM20A | 0.904 | 0.896 | 0.912 | 0.925 | 0.872 | 0.977 | 0.932 | 0.852 | 1 |
| IFI27_ISG15_IL1R2_FAM20A | 0.904 | 0.896 | 0.911 | 0.921 | 0.867 | 0.974 | 0.914 | 0.826 | 1 |
| RSAD2_IFI27_IL1R2_FAM20A | 0.904 | 0.896 | 0.912 | 0.925 | 0.872 | 0.977 | 0.927 | 0.844 | 1 |
| IFI27_ISG15_HERC6_OLAH | 0.904 | 0.896 | 0.912 | 0.919 | 0.863 | 0.975 | 0.938 | 0.880 | 0.996 |
| IFI27_SLC1A2_OLAH_FAM20A | 0.904 | 0.895 | 0.912 | 0.925 | 0.870 | 0.981 | 0.924 | 0.846 | 1 |
| IFI27_HERC6_IL1R2_OLAH | 0.903 | 0.896 | 0.911 | 0.924 | 0.873 | 0.975 | 0.943 | 0.890 | 0.997 |
| IFI27_IL1R2_OLAH_MMP8 | 0.903 | 0.896 | 0.911 | 0.922 | 0.870 | 0.974 | 0.937 | 0.878 | 0.996 |
| IFI27_IFIT1_HERC6_OLAH | 0.903 | 0.895 | 0.911 | 0.922 | 0.868 | 0.976 | 0.939 | 0.882 | 0.996 |
| IFI27_OLAH_RETN_MMP8 | 0.903 | 0.894 | 0.912 | 0.920 | 0.861 | 0.978 | 0.948 | 0.889 | 1 |
| IFI27_ISG15_IL1R2_OLAH | 0.903 | 0.895 | 0.910 | 0.922 | 0.870 | 0.975 | 0.935 | 0.873 | 0.997 |
| IFI27_IL1R2_FAM20A_RETN | 0.903 | 0.895 | 0.911 | 0.922 | 0.870 | 0.974 | 0.912 | 0.822 | 1 |
| IFI27_ISG15_OLAH_MMP8 | 0.902 | 0.894 | 0.910 | 0.916 | 0.859 | 0.973 | 0.934 | 0.871 | 0.996 |
| IFI27_IFI44L_SIGLEC1_OLAH | 0.902 | 0.894 | 0.910 | 0.920 | 0.864 | 0.976 | 0.948 | 0.896 | 0.999 |
| IFI27_HERC6_OLAH_MMP8 | 0.902 | 0.894 | 0.910 | 0.918 | 0.861 | 0.975 | 0.941 | 0.886 | 0.997 |
| SIGLEC1_IL1R2_FAM20A_MMP8 | 0.902 | 0.893 | 0.910 | 0.917 | 0.858 | 0.976 | 0.961 | 0.900 | 1 |
| IFI27_HERC6_FAM20A_RETN | 0.902 | 0.893 | 0.911 | 0.919 | 0.859 | 0.979 | 0.940 | 0.862 | 1 |
| IFI27_SIGLEC1_IL1R2_OLAH | 0.902 | 0.894 | 0.909 | 0.921 | 0.869 | 0.973 | 0.942 | 0.888 | 0.997 |
| IFI27_SIGLEC1_HERC6_OLAH | 0.901 | 0.893 | 0.910 | 0.917 | 0.859 | 0.975 | 0.942 | 0.887 | 0.997 |
| IFI27_IFI44L_IL1R2_OLAH | 0.901 | 0.893 | 0.909 | 0.922 | 0.869 | 0.974 | 0.938 | 0.879 | 0.997 |
| IFI27_HERC6_OLAH_RETN | 0.901 | 0.893 | 0.909 | 0.917 | 0.858 | 0.976 | 0.949 | 0.895 | 1 |
| IFI27_SIGLEC1_SLC1A2_OLAH | 0.901 | 0.893 | 0.909 | 0.927 | 0.875 | 0.979 | 0.940 | 0.881 | 1.000 |
| IFI27_SIGLEC1_OLAH_MMP8 | 0.901 | 0.893 | 0.909 | 0.917 | 0.859 | 0.975 | 0.945 | 0.890 | 0.999 |
| IFI27_SLC1A2_OLAH_MMP8 | 0.901 | 0.892 | 0.909 | 0.926 | 0.874 | 0.978 | 0.930 | 0.864 | 0.997 |
| IFI27_SIGLEC1_ISG15_OLAH | 0.901 | 0.893 | 0.909 | 0.915 | 0.857 | 0.974 | 0.942 | 0.886 | 0.999 |
| IFI27_OAS1_HERC6_RETN | 0.901 | 0.892 | 0.910 | 0.915 | 0.854 | 0.976 | 0.932 | 0.860 | 1 |
| IFI27_IFIT1_SIGLEC1_FAM20A | 0.901 | 0.892 | 0.909 | 0.914 | 0.849 | 0.978 | 0.948 | 0.878 | 1 |
| IFI27_IFI44L_OLAH_MMP8 | 0.901 | 0.892 | 0.909 | 0.916 | 0.859 | 0.974 | 0.937 | 0.876 | 0.997 |
| IFI27_ISG15_SLC1A2_OLAH | 0.901 | 0.893 | 0.909 | 0.926 | 0.874 | 0.977 | 0.923 | 0.850 | 0.995 |
| IFI27_ISG15_OLAH_RETN | 0.901 | 0.892 | 0.909 | 0.917 | 0.859 | 0.975 | 0.939 | 0.874 | 1 |
| IFI27_IFIT1_SIGLEC1_OLAH | 0.901 | 0.893 | 0.909 | 0.918 | 0.861 | 0.974 | 0.941 | 0.885 | 0.997 |
| IFI27_SLC1A2_IL1R2_OLAH | 0.901 | 0.893 | 0.908 | 0.927 | 0.877 | 0.977 | 0.926 | 0.856 | 0.996 |
| IFI27_IFIT1_IL1R2_OLAH | 0.900 | 0.892 | 0.908 | 0.921 | 0.869 | 0.974 | 0.941 | 0.886 | 0.997 |
| IFI27_IFI44L_SIGLEC1_IL1R2 | 0.900 | 0.893 | 0.907 | 0.919 | 0.869 | 0.968 | 0.943 | 0.893 | 0.994 |
| IFI27_IL1R2_OLAH_RETN | 0.900 | 0.892 | 0.908 | 0.918 | 0.863 | 0.974 | 0.941 | 0.879 | 1 |
| IFI27_IFI44L_SLC1A2_OLAH | 0.900 | 0.892 | 0.909 | 0.927 | 0.875 | 0.978 | 0.932 | 0.866 | 0.997 |
| RSAD2_IFI27_SLC1A2_OLAH | 0.900 | 0.892 | 0.908 | 0.926 | 0.875 | 0.978 | 0.932 | 0.866 | 0.997 |
| IFI27_IFIT1_SLC1A2_OLAH | 0.900 | 0.892 | 0.908 | 0.926 | 0.874 | 0.979 | 0.935 | 0.871 | 0.998 |
| RSAD2_IFI27_IL1R2_OLAH | 0.900 | 0.892 | 0.908 | 0.921 | 0.868 | 0.974 | 0.941 | 0.886 | 0.997 |
| IFI27_IFIT1_OLAH_MMP8 | 0.900 | 0.891 | 0.908 | 0.916 | 0.858 | 0.974 | 0.939 | 0.881 | 0.998 |
| IFI27_OAS1_IL1R2_OLAH | 0.900 | 0.892 | 0.908 | 0.922 | 0.869 | 0.974 | 0.941 | 0.887 | 0.996 |
| IFI27_HERC6_SLC1A2_OLAH | 0.900 | 0.891 | 0.908 | 0.928 | 0.878 | 0.979 | 0.936 | 0.874 | 0.998 |
| IFI27_SIGLEC1_IL1R2_RETN | 0.899 | 0.892 | 0.907 | 0.920 | 0.866 | 0.973 | 0.958 | 0.913 | 1 |
| IFI27_OAS1_IFIT1_OLAH | 0.899 | 0.891 | 0.907 | 0.916 | 0.858 | 0.974 | 0.938 | 0.877 | 0.999 |
| IFI27_IFI44L_HERC6_IL1R2 | 0.899 | 0.892 | 0.907 | 0.918 | 0.870 | 0.966 | 0.929 | 0.870 | 0.988 |
| IFI27_OAS1_SIGLEC1_IL1R2 | 0.899 | 0.892 | 0.907 | 0.919 | 0.870 | 0.968 | 0.950 | 0.903 | 0.997 |
| IFI27_SIGLEC1_HERC6_IL1R2 | 0.899 | 0.892 | 0.907 | 0.921 | 0.872 | 0.969 | 0.949 | 0.902 | 0.996 |
| IFI27_IFI44L_ISG15_OLAH | 0.899 | 0.891 | 0.907 | 0.917 | 0.859 | 0.974 | 0.934 | 0.871 | 0.996 |
| IFI27_OAS1_SIGLEC1_OLAH | 0.899 | 0.891 | 0.907 | 0.918 | 0.861 | 0.974 | 0.943 | 0.888 | 0.999 |
| IFI27_SIGLEC1_IL1R2_MMP8 | 0.899 | 0.892 | 0.906 | 0.921 | 0.873 | 0.969 | 0.950 | 0.903 | 0.997 |
| RSAD2_IFI27_SIGLEC1_OLAH | 0.899 | 0.891 | 0.907 | 0.919 | 0.863 | 0.975 | 0.941 | 0.885 | 0.998 |
| IFI27_IFIT1_ISG15_OLAH | 0.899 | 0.891 | 0.907 | 0.914 | 0.854 | 0.973 | 0.935 | 0.873 | 0.996 |
| IFI27_OAS1_OLAH_MMP8 | 0.899 | 0.891 | 0.907 | 0.916 | 0.858 | 0.974 | 0.937 | 0.877 | 0.997 |
| IFI27_OAS1_HERC6_IL1R2 | 0.899 | 0.892 | 0.906 | 0.918 | 0.870 | 0.966 | 0.929 | 0.870 | 0.988 |
| IFI27_SIGLEC1_OLAH_RETN | 0.899 | 0.890 | 0.907 | 0.916 | 0.856 | 0.976 | 0.951 | 0.896 | 1 |
| IFI27_IFI44L_OLAH_RETN | 0.899 | 0.890 | 0.907 | 0.914 | 0.855 | 0.973 | 0.941 | 0.878 | 1 |
| RSAD2_IFI27_OLAH_MMP8 | 0.899 | 0.891 | 0.907 | 0.916 | 0.857 | 0.974 | 0.938 | 0.879 | 0.997 |
| IFI27_IFIT1_SIGLEC1_IL1R2 | 0.899 | 0.891 | 0.906 | 0.919 | 0.870 | 0.968 | 0.950 | 0.903 | 0.997 |
| IFI27_OAS1_SLC1A2_OLAH | 0.899 | 0.890 | 0.907 | 0.926 | 0.874 | 0.978 | 0.928 | 0.860 | 0.997 |
| RSAD2_IFI27_SIGLEC1_IL1R2 | 0.899 | 0.891 | 0.906 | 0.919 | 0.870 | 0.968 | 0.948 | 0.899 | 0.997 |
| IFI27_IFIT1_OLAH_RETN | 0.899 | 0.890 | 0.907 | 0.915 | 0.855 | 0.974 | 0.945 | 0.884 | 1 |
| IFI27_SIGLEC1_ISG15_IL1R2 | 0.899 | 0.892 | 0.906 | 0.920 | 0.871 | 0.969 | 0.947 | 0.897 | 0.996 |
| IFI27_HERC6_IL1R2_RETN | 0.899 | 0.891 | 0.906 | 0.919 | 0.866 | 0.973 | 0.938 | 0.880 | 0.996 |
| IFI27_SLC1A2_OLAH_RETN | 0.898 | 0.890 | 0.907 | 0.925 | 0.873 | 0.977 | 0.933 | 0.864 | 1 |
| RSAD2_IFI27_HERC6_IL1R2 | 0.898 | 0.891 | 0.906 | 0.918 | 0.869 | 0.966 | 0.932 | 0.873 | 0.990 |
| IFI27_OAS1_OLAH_RETN | 0.898 | 0.890 | 0.906 | 0.915 | 0.856 | 0.974 | 0.942 | 0.882 | 1 |
| IFI27_SIGLEC1_FAM20A_RETN | 0.898 | 0.889 | 0.907 | 0.913 | 0.850 | 0.976 | 0.952 | 0.880 | 1 |
| IFI27_IFI44L_IL1R2_RETN | 0.898 | 0.890 | 0.906 | 0.920 | 0.868 | 0.972 | 0.938 | 0.877 | 0.999 |
| RSAD2_IFI27_SIGLEC1_FAM20A | 0.898 | 0.889 | 0.907 | 0.914 | 0.850 | 0.978 | 0.948 | 0.878 | 1 |
| RSAD2_IFI27_IFIT1_OLAH | 0.898 | 0.890 | 0.906 | 0.916 | 0.857 | 0.974 | 0.938 | 0.877 | 0.999 |
| RSAD2_IFI27_OAS1_OLAH | 0.898 | 0.890 | 0.906 | 0.917 | 0.859 | 0.974 | 0.938 | 0.879 | 0.997 |
| IFI27_SLC1A2_IL1R2_FAM20A | 0.898 | 0.889 | 0.906 | 0.922 | 0.874 | 0.971 | 0.905 | 0.810 | 1 |
| IFI27_IFIT1_HERC6_IL1R2 | 0.898 | 0.890 | 0.905 | 0.915 | 0.865 | 0.965 | 0.938 | 0.884 | 0.992 |
| IFI27_IFIT1_IFI44L_OLAH | 0.897 | 0.889 | 0.906 | 0.916 | 0.858 | 0.975 | 0.939 | 0.881 | 0.998 |
| RSAD2_IFI27_ISG15_OLAH | 0.897 | 0.889 | 0.905 | 0.915 | 0.856 | 0.974 | 0.936 | 0.875 | 0.996 |
| IFI27_OAS1_SIGLEC1_FAM20A | 0.897 | 0.888 | 0.906 | 0.910 | 0.845 | 0.976 | 0.948 | 0.879 | 1 |
| IFI27_OAS1_ISG15_OLAH | 0.897 | 0.889 | 0.905 | 0.916 | 0.858 | 0.974 | 0.936 | 0.877 | 0.995 |
| RSAD2_IFI27_OLAH_RETN | 0.897 | 0.889 | 0.905 | 0.914 | 0.854 | 0.973 | 0.945 | 0.885 | 1 |
| IFI27_IL1R2_RETN_MMP8 | 0.897 | 0.889 | 0.905 | 0.921 | 0.870 | 0.972 | 0.921 | 0.846 | 0.995 |
| IFI27_ISG15_HERC6_IL1R2 | 0.897 | 0.889 | 0.904 | 0.918 | 0.869 | 0.966 | 0.928 | 0.869 | 0.988 |
| IFI27_IFIT1_IL1R2_RETN | 0.897 | 0.889 | 0.904 | 0.917 | 0.864 | 0.971 | 0.946 | 0.892 | 0.999 |
| IFI27_OAS1_IFI44L_OLAH | 0.896 | 0.888 | 0.905 | 0.918 | 0.861 | 0.975 | 0.938 | 0.879 | 0.997 |
| IFIT1_SIGLEC1_HERC6_FAM20A | 0.896 | 0.888 | 0.905 | 0.911 | 0.849 | 0.972 | 0.937 | 0.865 | 1 |
| IFI27_HERC6_IL1R2_MMP8 | 0.896 | 0.889 | 0.904 | 0.916 | 0.867 | 0.965 | 0.933 | 0.875 | 0.990 |
| IFI27_ISG15_IL1R2_RETN | 0.896 | 0.888 | 0.904 | 0.919 | 0.866 | 0.971 | 0.932 | 0.865 | 0.998 |
| RSAD2_IFI27_IFI44L_OLAH | 0.896 | 0.888 | 0.904 | 0.917 | 0.860 | 0.974 | 0.940 | 0.883 | 0.998 |
| RSAD2_IFI27_HERC6_RETN | 0.896 | 0.887 | 0.905 | 0.913 | 0.853 | 0.974 | 0.940 | 0.878 | 1 |
| IFI27_IFIT1_IL1R2_MMP8 | 0.896 | 0.888 | 0.903 | 0.916 | 0.865 | 0.966 | 0.942 | 0.890 | 0.995 |
| IFI27_IFI44L_IL1R2_MMP8 | 0.896 | 0.888 | 0.903 | 0.912 | 0.861 | 0.963 | 0.932 | 0.873 | 0.991 |
| IFI27_OAS1_IFIT1_IL1R2 | 0.896 | 0.888 | 0.903 | 0.915 | 0.864 | 0.966 | 0.943 | 0.890 | 0.997 |
| IFI27_IFIT1_IFI44L_IL1R2 | 0.895 | 0.888 | 0.903 | 0.915 | 0.863 | 0.967 | 0.938 | 0.883 | 0.993 |
| IFI27_OAS1_IL1R2_RETN | 0.895 | 0.888 | 0.903 | 0.918 | 0.866 | 0.971 | 0.938 | 0.879 | 0.997 |
| RSAD2_IFI27_IL1R2_RETN | 0.895 | 0.887 | 0.903 | 0.919 | 0.865 | 0.972 | 0.940 | 0.882 | 0.998 |
| RSAD2_IFI27_IFIT1_IL1R2 | 0.895 | 0.887 | 0.902 | 0.916 | 0.865 | 0.966 | 0.940 | 0.887 | 0.994 |
| IFI27_SIGLEC1_SLC1A2_IL1R2 | 0.895 | 0.887 | 0.902 | 0.924 | 0.878 | 0.970 | 0.957 | 0.912 | 1 |
| SIGLEC1_HERC6_IL1R2_FAM20A | 0.895 | 0.886 | 0.903 | 0.912 | 0.849 | 0.974 | 0.958 | 0.897 | 1 |
| IFI27_IFI44L_SLC1A2_IL1R2 | 0.894 | 0.887 | 0.902 | 0.922 | 0.876 | 0.967 | 0.932 | 0.867 | 0.996 |
| IFI27_IFI44L_ISG15_IL1R2 | 0.894 | 0.887 | 0.902 | 0.915 | 0.866 | 0.965 | 0.937 | 0.880 | 0.994 |
| IFI27_IFI44L_SIGLEC1_FAM20A | 0.894 | 0.885 | 0.903 | 0.910 | 0.844 | 0.975 | 0.948 | 0.873 | 1 |
| IFI27_ISG15_IL1R2_MMP8 | 0.894 | 0.887 | 0.901 | 0.913 | 0.863 | 0.963 | 0.929 | 0.869 | 0.990 |
| RSAD2_IFI27_OAS1_IL1R2 | 0.894 | 0.886 | 0.901 | 0.915 | 0.865 | 0.966 | 0.939 | 0.884 | 0.994 |
| IFI27_SIGLEC1_ISG15_FAM20A | 0.894 | 0.884 | 0.903 | 0.904 | 0.835 | 0.973 | 0.948 | 0.873 | 1 |
| RSAD2_IFI27_IL1R2_MMP8 | 0.894 | 0.886 | 0.901 | 0.916 | 0.865 | 0.966 | 0.937 | 0.881 | 0.993 |
| IFI27_IFIT1_ISG15_IL1R2 | 0.893 | 0.886 | 0.901 | 0.915 | 0.864 | 0.966 | 0.941 | 0.888 | 0.994 |
| IFI27_IFIT1_HERC6_RETN | 0.893 | 0.884 | 0.902 | 0.907 | 0.844 | 0.971 | 0.941 | 0.878 | 1 |
| IFI27_IFI44L_FAM20A_RETN | 0.893 | 0.883 | 0.903 | 0.910 | 0.846 | 0.974 | 0.943 | 0.866 | 1 |
| IFI27_IFI44L_HERC6_RETN | 0.893 | 0.884 | 0.902 | 0.910 | 0.848 | 0.971 | 0.939 | 0.873 | 1 |
| IFIT1_SIGLEC1_IL1R2_FAM20A | 0.893 | 0.884 | 0.901 | 0.913 | 0.850 | 0.975 | 0.962 | 0.914 | 1 |
| RSAD2_IFI27_IFI44L_IL1R2 | 0.893 | 0.885 | 0.900 | 0.915 | 0.865 | 0.966 | 0.935 | 0.878 | 0.991 |
| IFI27_OAS1_IL1R2_MMP8 | 0.893 | 0.885 | 0.900 | 0.915 | 0.864 | 0.965 | 0.930 | 0.871 | 0.989 |
| IFI27_OAS1_IFI44L_IL1R2 | 0.893 | 0.885 | 0.900 | 0.914 | 0.864 | 0.965 | 0.932 | 0.873 | 0.990 |
| IFI27_OAS1_SIGLEC1_RETN | 0.893 | 0.884 | 0.901 | 0.909 | 0.848 | 0.971 | 0.953 | 0.892 | 1 |
| IFI44L_SIGLEC1_IL1R2_FAM20A | 0.893 | 0.884 | 0.901 | 0.910 | 0.847 | 0.973 | 0.962 | 0.911 | 1 |
| OAS1_SIGLEC1_IL1R2_FAM20A | 0.892 | 0.883 | 0.901 | 0.910 | 0.847 | 0.973 | 0.962 | 0.910 | 1 |
| IFI27_SLC1A2_IL1R2_RETN | 0.892 | 0.884 | 0.900 | 0.921 | 0.873 | 0.969 | 0.916 | 0.835 | 0.998 |
| RSAD2_SIGLEC1_IL1R2_FAM20A | 0.892 | 0.883 | 0.900 | 0.909 | 0.846 | 0.973 | 0.963 | 0.912 | 1 |
| SIGLEC1_ISG15_IL1R2_FAM20A | 0.892 | 0.883 | 0.900 | 0.908 | 0.845 | 0.972 | 0.963 | 0.912 | 1 |
| SIGLEC1_IL1R2_OLAH_FAM20A | 0.892 | 0.883 | 0.900 | 0.910 | 0.847 | 0.973 | 0.963 | 0.912 | 1 |
| IFI27_IFIT1_SLC1A2_IL1R2 | 0.892 | 0.884 | 0.899 | 0.922 | 0.876 | 0.968 | 0.936 | 0.875 | 0.997 |
| IFI27_IFIT1_SIGLEC1_RETN | 0.891 | 0.882 | 0.900 | 0.908 | 0.846 | 0.971 | 0.955 | 0.900 | 1 |
| IFI27_SIGLEC1_HERC6_FAM20A | 0.891 | 0.882 | 0.901 | 0.905 | 0.837 | 0.973 | 0.951 | 0.876 | 1 |
| RSAD2_IFI27_ISG15_IL1R2 | 0.891 | 0.884 | 0.898 | 0.916 | 0.866 | 0.966 | 0.934 | 0.876 | 0.991 |
| IFI27_OAS1_ISG15_IL1R2 | 0.891 | 0.883 | 0.898 | 0.914 | 0.863 | 0.964 | 0.932 | 0.873 | 0.990 |
| RSAD2_IFI27_SLC1A2_IL1R2 | 0.891 | 0.883 | 0.898 | 0.921 | 0.875 | 0.967 | 0.932 | 0.867 | 0.996 |
| SIGLEC1_IL1R2_FAM20A_RETN | 0.891 | 0.882 | 0.900 | 0.907 | 0.844 | 0.971 | 0.962 | 0.907 | 1 |
| IFI27_SIGLEC1_FAM20A_MMP8 | 0.890 | 0.881 | 0.900 | 0.904 | 0.836 | 0.972 | 0.946 | 0.867 | 1 |
| IFI27_ISG15_FAM20A_RETN | 0.890 | 0.881 | 0.900 | 0.909 | 0.848 | 0.969 | 0.934 | 0.853 | 1 |
| RSAD2_IFI27_SIGLEC1_RETN | 0.890 | 0.881 | 0.899 | 0.909 | 0.848 | 0.970 | 0.953 | 0.896 | 1 |
| IFI27_HERC6_SLC1A2_IL1R2 | 0.890 | 0.882 | 0.898 | 0.920 | 0.875 | 0.966 | 0.918 | 0.845 | 0.992 |
| IFI27_ISG15_HERC6_RETN | 0.890 | 0.880 | 0.899 | 0.907 | 0.844 | 0.971 | 0.951 | 0.893 | 1 |
| IFI27_SIGLEC1_HERC6_RETN | 0.890 | 0.880 | 0.899 | 0.905 | 0.840 | 0.971 | 0.963 | 0.917 | 1 |
| IFI27_IFIT1_FAM20A_RETN | 0.890 | 0.880 | 0.899 | 0.910 | 0.848 | 0.972 | 0.942 | 0.865 | 1 |
| IFI27_IFI44L_SIGLEC1_RETN | 0.889 | 0.881 | 0.898 | 0.907 | 0.844 | 0.970 | 0.954 | 0.898 | 1 |
| IFIT1_IFI44L_SIGLEC1_FAM20A | 0.889 | 0.881 | 0.898 | 0.901 | 0.832 | 0.970 | 0.939 | 0.875 | 1 |
| SIGLEC1_SLC1A2_IL1R2_FAM20A | 0.889 | 0.881 | 0.898 | 0.916 | 0.862 | 0.970 | 0.954 | 0.890 | 1 |
| IFI27_OAS1_SLC1A2_IL1R2 | 0.889 | 0.881 | 0.897 | 0.922 | 0.876 | 0.967 | 0.923 | 0.853 | 0.992 |
| SIGLEC1_SLC1A2_OLAH_FAM20A | 0.889 | 0.880 | 0.898 | 0.913 | 0.855 | 0.971 | 0.950 | 0.890 | 1 |
| ISG15_HERC6_IL1R2_FAM20A | 0.889 | 0.880 | 0.898 | 0.907 | 0.847 | 0.966 | 0.932 | 0.849 | 1 |
| IFI27_ISG15_SLC1A2_IL1R2 | 0.889 | 0.881 | 0.896 | 0.918 | 0.871 | 0.965 | 0.918 | 0.844 | 0.993 |
| IFI27_SLC1A2_IL1R2_MMP8 | 0.888 | 0.881 | 0.896 | 0.919 | 0.874 | 0.965 | 0.905 | 0.822 | 0.989 |
| OAS1_IFIT1_SIGLEC1_FAM20A | 0.888 | 0.879 | 0.897 | 0.899 | 0.829 | 0.970 | 0.947 | 0.880 | 1 |
| IFI27_OAS1_FAM20A_RETN | 0.888 | 0.878 | 0.897 | 0.910 | 0.848 | 0.971 | 0.942 | 0.865 | 1 |
| IFI44L_SIGLEC1_SLC1A2_OLAH | 0.888 | 0.879 | 0.896 | 0.912 | 0.856 | 0.967 | 0.950 | 0.902 | 0.998 |
| IFIT1_SIGLEC1_OLAH_FAM20A | 0.887 | 0.878 | 0.896 | 0.901 | 0.832 | 0.970 | 0.948 | 0.893 | 1 |
| SIGLEC1_OLAH_FAM20A_MMP8 | 0.887 | 0.877 | 0.897 | 0.901 | 0.832 | 0.970 | 0.965 | 0.920 | 1 |
| IFI44L_HERC6_IL1R2_FAM20A | 0.887 | 0.878 | 0.896 | 0.906 | 0.845 | 0.967 | 0.936 | 0.856 | 1 |
| IFI27_SIGLEC1_ISG15_RETN | 0.887 | 0.877 | 0.896 | 0.904 | 0.838 | 0.969 | 0.957 | 0.903 | 1 |
| IFIT1_SIGLEC1_SLC1A2_OLAH | 0.887 | 0.878 | 0.895 | 0.913 | 0.857 | 0.968 | 0.947 | 0.897 | 0.997 |
| IFI27_HERC6_RETN_MMP8 | 0.886 | 0.877 | 0.896 | 0.905 | 0.841 | 0.969 | 0.947 | 0.886 | 1 |
| HERC6_IL1R2_OLAH_FAM20A | 0.886 | 0.877 | 0.895 | 0.907 | 0.847 | 0.966 | 0.930 | 0.853 | 1 |
| IFI27_HERC6_FAM20A_MMP8 | 0.886 | 0.877 | 0.896 | 0.906 | 0.844 | 0.968 | 0.932 | 0.851 | 1 |
| RSAD2_IFI27_FAM20A_RETN | 0.886 | 0.877 | 0.896 | 0.908 | 0.846 | 0.971 | 0.942 | 0.864 | 1 |
| OAS1_HERC6_IL1R2_FAM20A | 0.886 | 0.877 | 0.895 | 0.905 | 0.843 | 0.967 | 0.930 | 0.847 | 1 |
| IFIT1_HERC6_IL1R2_FAM20A | 0.886 | 0.877 | 0.895 | 0.903 | 0.840 | 0.965 | 0.936 | 0.854 | 1 |
| SIGLEC1_SLC1A2_OLAH_MMP8 | 0.886 | 0.877 | 0.895 | 0.912 | 0.856 | 0.967 | 0.957 | 0.914 | 0.999 |
| OAS1_SIGLEC1_SLC1A2_OLAH | 0.886 | 0.877 | 0.895 | 0.912 | 0.857 | 0.968 | 0.952 | 0.906 | 0.999 |
| RSAD2_SIGLEC1_SLC1A2_OLAH | 0.886 | 0.877 | 0.894 | 0.912 | 0.856 | 0.968 | 0.953 | 0.908 | 0.998 |
| IFIT1_SIGLEC1_ISG15_FAM20A | 0.886 | 0.877 | 0.894 | 0.896 | 0.826 | 0.967 | 0.939 | 0.873 | 1 |
| IFI27_SIGLEC1_SLC1A2_FAM20A | 0.886 | 0.876 | 0.895 | 0.911 | 0.848 | 0.973 | 0.950 | 0.874 | 1 |
| SIGLEC1_ISG15_SLC1A2_OLAH | 0.886 | 0.877 | 0.894 | 0.912 | 0.856 | 0.967 | 0.948 | 0.899 | 0.997 |
| IFI44L_SIGLEC1_HERC6_IL1R2 | 0.886 | 0.877 | 0.894 | 0.902 | 0.841 | 0.962 | 0.957 | 0.914 | 0.999 |
| IFI44L_SLC1A2_OLAH_FAM20A | 0.885 | 0.876 | 0.895 | 0.908 | 0.849 | 0.968 | 0.927 | 0.853 | 1 |
| IFIT1_SIGLEC1_HERC6_IL1R2 | 0.885 | 0.877 | 0.894 | 0.904 | 0.845 | 0.962 | 0.953 | 0.909 | 0.998 |
| RSAD2_IFIT1_SIGLEC1_FAM20A | 0.885 | 0.876 | 0.894 | 0.899 | 0.829 | 0.969 | 0.943 | 0.879 | 1 |
| SIGLEC1_HERC6_OLAH_FAM20A | 0.885 | 0.875 | 0.895 | 0.902 | 0.834 | 0.970 | 0.960 | 0.907 | 1 |
| RSAD2_SIGLEC1_HERC6_IL1R2 | 0.885 | 0.876 | 0.893 | 0.903 | 0.843 | 0.962 | 0.949 | 0.902 | 0.996 |
| IFIT1_SIGLEC1_FAM20A_MMP8 | 0.885 | 0.876 | 0.894 | 0.899 | 0.829 | 0.969 | 0.940 | 0.872 | 1 |
| OAS1_SIGLEC1_HERC6_IL1R2 | 0.885 | 0.876 | 0.893 | 0.903 | 0.845 | 0.962 | 0.957 | 0.914 | 0.999 |
| RSAD2_HERC6_IL1R2_FAM20A | 0.885 | 0.876 | 0.894 | 0.905 | 0.843 | 0.967 | 0.930 | 0.846 | 1 |
| SIGLEC1_SLC1A2_IL1R2_OLAH | 0.885 | 0.876 | 0.893 | 0.913 | 0.858 | 0.968 | 0.963 | 0.925 | 1 |
| SIGLEC1_ISG15_OLAH_FAM20A | 0.884 | 0.875 | 0.894 | 0.897 | 0.825 | 0.968 | 0.962 | 0.918 | 1 |
| HERC6_IL1R2_FAM20A_MMP8 | 0.884 | 0.876 | 0.893 | 0.909 | 0.852 | 0.966 | 0.920 | 0.828 | 1 |
| IFI44L_SIGLEC1_OLAH_FAM20A | 0.884 | 0.875 | 0.894 | 0.896 | 0.824 | 0.969 | 0.962 | 0.918 | 1 |
| SIGLEC1_HERC6_SLC1A2_OLAH | 0.884 | 0.875 | 0.894 | 0.912 | 0.856 | 0.968 | 0.952 | 0.907 | 0.997 |
| IFI27_SIGLEC1_SLC1A2_RETN | 0.884 | 0.875 | 0.893 | 0.909 | 0.848 | 0.971 | 0.960 | 0.907 | 1 |
| SIGLEC1_HERC6_IL1R2_MMP8 | 0.884 | 0.875 | 0.892 | 0.903 | 0.844 | 0.962 | 0.964 | 0.927 | 1 |
| IFI27_SIGLEC1_RETN_MMP8 | 0.884 | 0.874 | 0.893 | 0.904 | 0.840 | 0.969 | 0.962 | 0.912 | 1 |
| IFIT1_SIGLEC1_FAM20A_RETN | 0.884 | 0.874 | 0.893 | 0.898 | 0.828 | 0.968 | 0.941 | 0.878 | 1 |
| HERC6_IL1R2_FAM20A_RETN | 0.884 | 0.874 | 0.893 | 0.907 | 0.846 | 0.967 | 0.926 | 0.840 | 1 |
| OAS1_SIGLEC1_OLAH_FAM20A | 0.883 | 0.874 | 0.893 | 0.896 | 0.824 | 0.968 | 0.962 | 0.918 | 1 |
| IFIT1_SIGLEC1_SLC1A2_FAM20A | 0.883 | 0.874 | 0.892 | 0.907 | 0.847 | 0.968 | 0.938 | 0.865 | 1 |
| IFI44L_SLC1A2_IL1R2_FAM20A | 0.883 | 0.875 | 0.892 | 0.908 | 0.853 | 0.962 | 0.925 | 0.838 | 1 |
| SIGLEC1_FAM20A_RETN_MMP8 | 0.883 | 0.873 | 0.893 | 0.900 | 0.829 | 0.970 | 0.958 | 0.896 | 1 |
| SIGLEC1_HERC6_FAM20A_RETN | 0.883 | 0.873 | 0.893 | 0.898 | 0.827 | 0.968 | 0.955 | 0.890 | 1 |
| IFI27_ISG15_HERC6_FAM20A | 0.883 | 0.873 | 0.893 | 0.901 | 0.837 | 0.965 | 0.936 | 0.856 | 1 |
| IFI27_FAM20A_RETN_MMP8 | 0.883 | 0.873 | 0.894 | 0.906 | 0.844 | 0.968 | 0.923 | 0.832 | 1 |
| IFI44L_SIGLEC1_IL1R2_MMP8 | 0.883 | 0.875 | 0.891 | 0.902 | 0.843 | 0.961 | 0.955 | 0.912 | 0.999 |
| SIGLEC1_SLC1A2_OLAH_RETN | 0.883 | 0.874 | 0.892 | 0.913 | 0.857 | 0.969 | 0.949 | 0.899 | 0.998 |
| IFI27_IFIT1_HERC6_FAM20A | 0.883 | 0.873 | 0.893 | 0.903 | 0.841 | 0.966 | 0.934 | 0.851 | 1 |
| IFI27_HERC6_SLC1A2_RETN | 0.883 | 0.873 | 0.892 | 0.910 | 0.849 | 0.971 | 0.942 | 0.874 | 1 |
| IFI27_IFI44L_ISG15_RETN | 0.883 | 0.873 | 0.892 | 0.905 | 0.841 | 0.969 | 0.957 | 0.901 | 1 |
| SIGLEC1_ISG15_HERC6_IL1R2 | 0.883 | 0.874 | 0.891 | 0.902 | 0.841 | 0.963 | 0.953 | 0.909 | 0.998 |
| RSAD2_IFI27_HERC6_FAM20A | 0.883 | 0.873 | 0.892 | 0.905 | 0.844 | 0.966 | 0.928 | 0.845 | 1 |
| IFI27_IFI44L_HERC6_FAM20A | 0.882 | 0.873 | 0.892 | 0.898 | 0.832 | 0.964 | 0.937 | 0.857 | 1 |
| RSAD2_SIGLEC1_OLAH_FAM20A | 0.882 | 0.873 | 0.892 | 0.896 | 0.825 | 0.968 | 0.962 | 0.919 | 1 |
| IFI27_OAS1_IFIT1_RETN | 0.882 | 0.873 | 0.892 | 0.905 | 0.841 | 0.968 | 0.950 | 0.888 | 1 |
| SIGLEC1_OLAH_FAM20A_RETN | 0.882 | 0.872 | 0.892 | 0.896 | 0.823 | 0.968 | 0.963 | 0.920 | 1 |
| RSAD2_SIGLEC1_HERC6_FAM20A | 0.882 | 0.873 | 0.891 | 0.899 | 0.834 | 0.965 | 0.941 | 0.872 | 1 |
| SIGLEC1_HERC6_IL1R2_OLAH | 0.882 | 0.873 | 0.891 | 0.903 | 0.842 | 0.964 | 0.959 | 0.918 | 1.000 |
| IFI27_OAS1_HERC6_FAM20A | 0.882 | 0.872 | 0.892 | 0.903 | 0.840 | 0.965 | 0.932 | 0.848 | 1 |
| IFI44L_SIGLEC1_SLC1A2_IL1R2 | 0.882 | 0.874 | 0.890 | 0.907 | 0.851 | 0.963 | 0.964 | 0.927 | 1 |
| SIGLEC1_IL1R2_OLAH_MMP8 | 0.882 | 0.873 | 0.890 | 0.905 | 0.847 | 0.963 | 0.963 | 0.925 | 1 |
| IFI44L_ISG15_IL1R2_FAM20A | 0.882 | 0.873 | 0.890 | 0.901 | 0.839 | 0.964 | 0.941 | 0.871 | 1 |
| OAS1_IL1R2_OLAH_FAM20A | 0.881 | 0.873 | 0.890 | 0.901 | 0.835 | 0.967 | 0.936 | 0.860 | 1 |
| HERC6_SLC1A2_IL1R2_FAM20A | 0.881 | 0.872 | 0.891 | 0.912 | 0.860 | 0.965 | 0.916 | 0.818 | 1 |
| SIGLEC1_SLC1A2_IL1R2_MMP8 | 0.881 | 0.873 | 0.889 | 0.910 | 0.855 | 0.964 | 0.966 | 0.930 | 1 |
| SIGLEC1_ISG15_SLC1A2_IL1R2 | 0.881 | 0.873 | 0.889 | 0.908 | 0.853 | 0.964 | 0.964 | 0.927 | 1 |
| SIGLEC1_HERC6_SLC1A2_IL1R2 | 0.881 | 0.873 | 0.890 | 0.908 | 0.853 | 0.963 | 0.961 | 0.922 | 1 |
| IFI27_IFIT1_ISG15_RETN | 0.881 | 0.872 | 0.891 | 0.904 | 0.841 | 0.968 | 0.953 | 0.896 | 1 |
| RSAD2_SIGLEC1_IL1R2_MMP8 | 0.881 | 0.873 | 0.890 | 0.903 | 0.844 | 0.962 | 0.965 | 0.929 | 1 |
| IFI44L_SIGLEC1_IL1R2_OLAH | 0.881 | 0.873 | 0.890 | 0.901 | 0.840 | 0.962 | 0.958 | 0.915 | 1 |
| IFI27_SLC1A2_FAM20A_RETN | 0.881 | 0.871 | 0.891 | 0.899 | 0.835 | 0.964 | 0.913 | 0.817 | 1 |
| IFIT1_IL1R2_FAM20A_MMP8 | 0.881 | 0.872 | 0.890 | 0.904 | 0.841 | 0.967 | 0.941 | 0.867 | 1 |
| IFI44L_SIGLEC1_ISG15_IL1R2 | 0.881 | 0.873 | 0.889 | 0.900 | 0.839 | 0.961 | 0.960 | 0.919 | 1 |
| OAS1_IFIT1_SIGLEC1_IL1R2 | 0.881 | 0.873 | 0.890 | 0.899 | 0.836 | 0.961 | 0.957 | 0.915 | 0.998 |
| IFI27_IFI44L_FAM20A_MMP8 | 0.881 | 0.871 | 0.891 | 0.899 | 0.832 | 0.966 | 0.935 | 0.856 | 1 |
| IFIT1_SIGLEC1_SLC1A2_IL1R2 | 0.881 | 0.873 | 0.889 | 0.908 | 0.853 | 0.963 | 0.964 | 0.927 | 1 |
| SIGLEC1_SLC1A2_IL1R2_RETN | 0.881 | 0.873 | 0.889 | 0.910 | 0.855 | 0.965 | 0.962 | 0.922 | 1 |
| IFI27_OAS1_IFI44L_RETN | 0.881 | 0.871 | 0.890 | 0.906 | 0.843 | 0.970 | 0.954 | 0.896 | 1 |
| OAS1_SIGLEC1_IL1R2_MMP8 | 0.881 | 0.872 | 0.889 | 0.902 | 0.843 | 0.962 | 0.963 | 0.925 | 1 |
| OAS1_IFIT1_IL1R2_FAM20A | 0.881 | 0.872 | 0.890 | 0.901 | 0.836 | 0.967 | 0.942 | 0.868 | 1 |
| RSAD2_SIGLEC1_SLC1A2_IL1R2 | 0.881 | 0.872 | 0.889 | 0.909 | 0.853 | 0.964 | 0.965 | 0.929 | 1 |
| IFIT1_SIGLEC1_HERC6_OLAH | 0.881 | 0.872 | 0.890 | 0.900 | 0.838 | 0.962 | 0.938 | 0.885 | 0.992 |
| HERC6_SLC1A2_OLAH_FAM20A | 0.881 | 0.871 | 0.890 | 0.911 | 0.856 | 0.967 | 0.929 | 0.849 | 1 |
| RSAD2_OAS1_SIGLEC1_FAM20A | 0.881 | 0.871 | 0.890 | 0.897 | 0.826 | 0.968 | 0.952 | 0.889 | 1 |
| OAS1_IL1R2_FAM20A_MMP8 | 0.881 | 0.872 | 0.889 | 0.903 | 0.841 | 0.966 | 0.934 | 0.853 | 1 |
| SIGLEC1_ISG15_FAM20A_RETN | 0.881 | 0.871 | 0.890 | 0.893 | 0.821 | 0.966 | 0.954 | 0.895 | 1 |
| SIGLEC1_ISG15_IL1R2_MMP8 | 0.881 | 0.873 | 0.889 | 0.903 | 0.844 | 0.962 | 0.962 | 0.923 | 1 |
| IFI27_IFI44L_RETN_MMP8 | 0.880 | 0.871 | 0.890 | 0.905 | 0.842 | 0.969 | 0.950 | 0.892 | 1 |
| IFI44L_SIGLEC1_SLC1A2_FAM20A | 0.880 | 0.871 | 0.890 | 0.901 | 0.837 | 0.966 | 0.949 | 0.877 | 1 |
| IFI44L_SLC1A2_OLAH_MMP8 | 0.880 | 0.871 | 0.889 | 0.906 | 0.848 | 0.964 | 0.937 | 0.882 | 0.991 |
| SIGLEC1_SLC1A2_FAM20A_MMP8 | 0.880 | 0.870 | 0.890 | 0.902 | 0.838 | 0.966 | 0.951 | 0.877 | 1 |
| IFIT1_SIGLEC1_IL1R2_MMP8 | 0.880 | 0.872 | 0.889 | 0.901 | 0.842 | 0.960 | 0.962 | 0.923 | 1 |
| IFIT1_SIGLEC1_HERC6_RETN | 0.880 | 0.871 | 0.890 | 0.893 | 0.823 | 0.963 | 0.925 | 0.865 | 0.985 |
| IFI44L_SIGLEC1_HERC6_FAM20A | 0.880 | 0.870 | 0.890 | 0.899 | 0.832 | 0.967 | 0.952 | 0.881 | 1 |
| SIGLEC1_ISG15_IL1R2_OLAH | 0.880 | 0.872 | 0.888 | 0.902 | 0.841 | 0.963 | 0.961 | 0.920 | 1 |
| SIGLEC1_ISG15_SLC1A2_FAM20A | 0.880 | 0.871 | 0.889 | 0.901 | 0.837 | 0.965 | 0.949 | 0.877 | 1 |
| OAS1_IFI44L_SIGLEC1_IL1R2 | 0.880 | 0.871 | 0.889 | 0.899 | 0.836 | 0.963 | 0.957 | 0.915 | 0.998 |
| RSAD2_OAS1_IL1R2_FAM20A | 0.880 | 0.871 | 0.889 | 0.898 | 0.832 | 0.965 | 0.936 | 0.858 | 1 |
| OAS1_SIGLEC1_IL1R2_OLAH | 0.880 | 0.871 | 0.889 | 0.900 | 0.837 | 0.963 | 0.955 | 0.912 | 0.999 |
| IFI44L_IL1R2_FAM20A_MMP8 | 0.880 | 0.871 | 0.888 | 0.901 | 0.840 | 0.963 | 0.936 | 0.855 | 1 |
| IFI27_IFIT1_IFI44L_RETN | 0.880 | 0.870 | 0.890 | 0.903 | 0.839 | 0.968 | 0.952 | 0.892 | 1 |
| OAS1_SLC1A2_OLAH_FAM20A | 0.880 | 0.870 | 0.889 | 0.912 | 0.855 | 0.970 | 0.939 | 0.868 | 1 |
| OAS1_SIGLEC1_SLC1A2_IL1R2 | 0.880 | 0.871 | 0.888 | 0.908 | 0.853 | 0.964 | 0.964 | 0.927 | 1 |
| SIGLEC1_HERC6_IL1R2_RETN | 0.880 | 0.870 | 0.889 | 0.897 | 0.831 | 0.964 | 0.960 | 0.919 | 1 |
| IFI44L_SLC1A2_IL1R2_OLAH | 0.880 | 0.871 | 0.889 | 0.907 | 0.851 | 0.963 | 0.929 | 0.868 | 0.991 |
| SIGLEC1_SLC1A2_FAM20A_RETN | 0.880 | 0.870 | 0.889 | 0.902 | 0.838 | 0.966 | 0.948 | 0.875 | 1 |
| RSAD2_IFI27_IFIT1_RETN | 0.880 | 0.870 | 0.889 | 0.905 | 0.842 | 0.969 | 0.951 | 0.891 | 1 |
| RSAD2_SIGLEC1_SLC1A2_FAM20A | 0.880 | 0.870 | 0.889 | 0.901 | 0.838 | 0.964 | 0.947 | 0.877 | 1 |
| SIGLEC1_HERC6_SLC1A2_FAM20A | 0.880 | 0.870 | 0.889 | 0.901 | 0.837 | 0.964 | 0.954 | 0.881 | 1 |
| HERC6_OLAH_FAM20A_RETN | 0.879 | 0.870 | 0.889 | 0.903 | 0.839 | 0.967 | 0.945 | 0.879 | 1 |
| RSAD2_OAS1_SIGLEC1_IL1R2 | 0.879 | 0.871 | 0.888 | 0.895 | 0.831 | 0.959 | 0.962 | 0.924 | 1 |
| IFI27_OAS1_HERC6_MMP8 | 0.879 | 0.871 | 0.888 | 0.899 | 0.840 | 0.957 | 0.891 | 0.809 | 0.973 |
| IFI44L_SIGLEC1_FAM20A_RETN | 0.879 | 0.870 | 0.889 | 0.894 | 0.823 | 0.966 | 0.955 | 0.898 | 1 |
| IFI27_OAS1_ISG15_RETN | 0.879 | 0.870 | 0.889 | 0.905 | 0.843 | 0.968 | 0.952 | 0.896 | 1 |
| SIGLEC1_IL1R2_RETN_MMP8 | 0.879 | 0.870 | 0.888 | 0.899 | 0.835 | 0.963 | 0.968 | 0.935 | 1 |
| IFIT1_HERC6_OLAH_FAM20A | 0.879 | 0.869 | 0.889 | 0.899 | 0.835 | 0.964 | 0.941 | 0.872 | 1 |
| IFIT1_SIGLEC1_IL1R2_OLAH | 0.879 | 0.871 | 0.888 | 0.901 | 0.841 | 0.962 | 0.960 | 0.918 | 1 |
| IFI44L_IL1R2_OLAH_FAM20A | 0.879 | 0.870 | 0.888 | 0.897 | 0.832 | 0.962 | 0.951 | 0.896 | 1 |
| HERC6_OLAH_FAM20A_MMP8 | 0.879 | 0.869 | 0.889 | 0.899 | 0.835 | 0.963 | 0.942 | 0.875 | 1 |
| OAS1_IFI44L_IL1R2_FAM20A | 0.879 | 0.870 | 0.888 | 0.900 | 0.834 | 0.965 | 0.940 | 0.866 | 1 |
| RSAD2_IFI27_IFI44L_RETN | 0.879 | 0.869 | 0.888 | 0.906 | 0.842 | 0.970 | 0.957 | 0.902 | 1 |
| IFIT1_SIGLEC1_ISG15_IL1R2 | 0.879 | 0.870 | 0.887 | 0.900 | 0.840 | 0.961 | 0.961 | 0.921 | 1 |
| IFI27_IFIT1_RETN_MMP8 | 0.879 | 0.869 | 0.889 | 0.904 | 0.840 | 0.968 | 0.950 | 0.889 | 1 |
| IFIT1_ISG15_IL1R2_FAM20A | 0.879 | 0.870 | 0.888 | 0.903 | 0.840 | 0.965 | 0.951 | 0.887 | 1 |
| IFI27_IFI44L_SLC1A2_RETN | 0.879 | 0.869 | 0.888 | 0.903 | 0.840 | 0.967 | 0.946 | 0.880 | 1 |
| RSAD2_IFI27_OAS1_RETN | 0.879 | 0.869 | 0.888 | 0.901 | 0.836 | 0.966 | 0.950 | 0.889 | 1 |
| IFI44L_SLC1A2_OLAH_RETN | 0.879 | 0.869 | 0.888 | 0.907 | 0.848 | 0.965 | 0.929 | 0.870 | 0.988 |
| ISG15_HERC6_OLAH_FAM20A | 0.879 | 0.869 | 0.888 | 0.899 | 0.835 | 0.964 | 0.946 | 0.881 | 1 |
| OAS1_SIGLEC1_HERC6_FAM20A | 0.879 | 0.869 | 0.888 | 0.896 | 0.829 | 0.964 | 0.949 | 0.885 | 1 |
| RSAD2_SIGLEC1_IL1R2_OLAH | 0.879 | 0.869 | 0.888 | 0.899 | 0.836 | 0.963 | 0.960 | 0.919 | 1 |
| ISG15_SLC1A2_IL1R2_FAM20A | 0.879 | 0.870 | 0.887 | 0.905 | 0.849 | 0.962 | 0.916 | 0.826 | 1 |
| OAS1_SIGLEC1_FAM20A_RETN | 0.879 | 0.869 | 0.888 | 0.894 | 0.822 | 0.966 | 0.955 | 0.898 | 1 |
| IFI44L_HERC6_OLAH_FAM20A | 0.879 | 0.869 | 0.888 | 0.900 | 0.834 | 0.965 | 0.947 | 0.882 | 1 |
| IFI27_ISG15_RETN_MMP8 | 0.879 | 0.869 | 0.888 | 0.903 | 0.840 | 0.965 | 0.945 | 0.879 | 1 |
| RSAD2_IFIT1_SIGLEC1_IL1R2 | 0.878 | 0.870 | 0.887 | 0.897 | 0.835 | 0.960 | 0.961 | 0.922 | 1 |
| RSAD2_SLC1A2_OLAH_FAM20A | 0.878 | 0.869 | 0.888 | 0.908 | 0.849 | 0.966 | 0.932 | 0.858 | 1 |
| IFI44L_SIGLEC1_ISG15_FAM20A | 0.878 | 0.869 | 0.888 | 0.893 | 0.821 | 0.965 | 0.958 | 0.898 | 1 |
| IFI44L_SIGLEC1_IL1R2_RETN | 0.878 | 0.870 | 0.887 | 0.897 | 0.833 | 0.961 | 0.959 | 0.916 | 1 |
| RSAD2_IFI44L_SIGLEC1_IL1R2 | 0.878 | 0.870 | 0.887 | 0.896 | 0.831 | 0.961 | 0.960 | 0.920 | 1.000 |
| IFIT1_IFI44L_SIGLEC1_IL1R2 | 0.878 | 0.870 | 0.887 | 0.897 | 0.834 | 0.960 | 0.958 | 0.916 | 0.999 |
| IFI44L_ISG15_SLC1A2_OLAH | 0.878 | 0.869 | 0.887 | 0.906 | 0.848 | 0.963 | 0.933 | 0.876 | 0.989 |
| OAS1_ISG15_IL1R2_FAM20A | 0.878 | 0.869 | 0.887 | 0.896 | 0.829 | 0.962 | 0.936 | 0.857 | 1 |
| OAS1_SIGLEC1_SLC1A2_FAM20A | 0.878 | 0.869 | 0.888 | 0.901 | 0.838 | 0.965 | 0.948 | 0.877 | 1 |
| IFIT1_SLC1A2_IL1R2_FAM20A | 0.878 | 0.869 | 0.887 | 0.904 | 0.846 | 0.962 | 0.937 | 0.860 | 1 |
| IFI44L_SIGLEC1_FAM20A_MMP8 | 0.878 | 0.868 | 0.888 | 0.892 | 0.820 | 0.964 | 0.957 | 0.895 | 1 |
| SIGLEC1_ISG15_HERC6_FAM20A | 0.878 | 0.868 | 0.888 | 0.892 | 0.820 | 0.964 | 0.952 | 0.879 | 1 |
| ISG15_IL1R2_OLAH_FAM20A | 0.878 | 0.869 | 0.887 | 0.901 | 0.839 | 0.963 | 0.927 | 0.851 | 1 |
| SIGLEC1_ISG15_FAM20A_MMP8 | 0.878 | 0.868 | 0.888 | 0.889 | 0.816 | 0.963 | 0.958 | 0.896 | 1 |
| RSAD2_SIGLEC1_ISG15_IL1R2 | 0.878 | 0.869 | 0.886 | 0.897 | 0.834 | 0.960 | 0.962 | 0.924 | 1 |
| SIGLEC1_ISG15_IL1R2_RETN | 0.878 | 0.869 | 0.886 | 0.898 | 0.833 | 0.963 | 0.965 | 0.926 | 1 |
| SIGLEC1_IL1R2_OLAH_RETN | 0.878 | 0.869 | 0.887 | 0.898 | 0.833 | 0.963 | 0.960 | 0.918 | 1 |
| RSAD2_IFI27_IFI44L_FAM20A | 0.878 | 0.867 | 0.888 | 0.899 | 0.830 | 0.968 | 0.942 | 0.867 | 1 |
| RSAD2_IL1R2_OLAH_FAM20A | 0.877 | 0.868 | 0.887 | 0.900 | 0.834 | 0.967 | 0.947 | 0.879 | 1 |
| OAS1_SIGLEC1_ISG15_IL1R2 | 0.877 | 0.869 | 0.886 | 0.898 | 0.835 | 0.960 | 0.961 | 0.922 | 1.000 |
| OAS1_IFIT1_SIGLEC1_OLAH | 0.877 | 0.868 | 0.886 | 0.894 | 0.826 | 0.963 | 0.942 | 0.889 | 0.996 |
| RSAD2_SIGLEC1_FAM20A_RETN | 0.877 | 0.868 | 0.887 | 0.894 | 0.823 | 0.965 | 0.953 | 0.895 | 1 |
| RSAD2_SIGLEC1_FAM20A_MMP8 | 0.877 | 0.868 | 0.887 | 0.893 | 0.822 | 0.964 | 0.952 | 0.887 | 1 |
| IFI27_IFIT1_HERC6_MMP8 | 0.877 | 0.868 | 0.886 | 0.897 | 0.837 | 0.957 | 0.892 | 0.810 | 0.975 |
| SIGLEC1_HERC6_FAM20A_MMP8 | 0.877 | 0.867 | 0.887 | 0.893 | 0.821 | 0.965 | 0.952 | 0.879 | 1 |
| RSAD2_IFI27_ISG15_RETN | 0.877 | 0.868 | 0.887 | 0.904 | 0.840 | 0.968 | 0.950 | 0.890 | 1 |
| IFIT1_IFI44L_SLC1A2_OLAH | 0.877 | 0.868 | 0.886 | 0.907 | 0.849 | 0.964 | 0.934 | 0.878 | 0.990 |
| RSAD2_HERC6_OLAH_FAM20A | 0.877 | 0.868 | 0.887 | 0.900 | 0.836 | 0.964 | 0.942 | 0.875 | 1 |
| IFIT1_SIGLEC1_IL1R2_RETN | 0.877 | 0.868 | 0.886 | 0.897 | 0.833 | 0.961 | 0.958 | 0.914 | 1 |
| IFI27_IFIT1_SLC1A2_RETN | 0.877 | 0.867 | 0.886 | 0.902 | 0.838 | 0.966 | 0.945 | 0.875 | 1 |
| OAS1_HERC6_OLAH_FAM20A | 0.877 | 0.867 | 0.886 | 0.898 | 0.833 | 0.963 | 0.942 | 0.875 | 1 |
| IFI27_IFI44L_ISG15_FAM20A | 0.877 | 0.867 | 0.886 | 0.894 | 0.824 | 0.963 | 0.942 | 0.863 | 1 |
| OAS1_IL1R2_FAM20A_RETN | 0.877 | 0.867 | 0.886 | 0.899 | 0.833 | 0.965 | 0.933 | 0.853 | 1 |
| OAS1_SLC1A2_IL1R2_FAM20A | 0.877 | 0.868 | 0.885 | 0.905 | 0.848 | 0.962 | 0.926 | 0.839 | 1 |
| RSAD2_SIGLEC1_ISG15_FAM20A | 0.876 | 0.867 | 0.886 | 0.895 | 0.824 | 0.965 | 0.951 | 0.889 | 1 |
| IFI44L_SIGLEC1_HERC6_OLAH | 0.876 | 0.867 | 0.886 | 0.894 | 0.828 | 0.960 | 0.952 | 0.907 | 0.997 |
| IFIT1_SLC1A2_OLAH_FAM20A | 0.876 | 0.867 | 0.886 | 0.903 | 0.842 | 0.964 | 0.938 | 0.868 | 1 |
| RSAD2_SLC1A2_IL1R2_FAM20A | 0.876 | 0.868 | 0.885 | 0.901 | 0.843 | 0.958 | 0.930 | 0.842 | 1 |
| OAS1_SIGLEC1_FAM20A_MMP8 | 0.876 | 0.866 | 0.886 | 0.892 | 0.821 | 0.964 | 0.955 | 0.893 | 1 |
| OAS1_SIGLEC1_IL1R2_RETN | 0.876 | 0.867 | 0.885 | 0.897 | 0.831 | 0.963 | 0.957 | 0.913 | 1.000 |
| RSAD2_ISG15_IL1R2_FAM20A | 0.876 | 0.867 | 0.885 | 0.897 | 0.832 | 0.963 | 0.941 | 0.867 | 1 |
| OAS1_IFIT1_HERC6_IL1R2 | 0.876 | 0.867 | 0.885 | 0.895 | 0.833 | 0.958 | 0.941 | 0.889 | 0.993 |
| IFI27_HERC6_SLC1A2_FAM20A | 0.876 | 0.866 | 0.886 | 0.904 | 0.846 | 0.963 | 0.933 | 0.850 | 1 |
| IFI27_ISG15_SLC1A2_RETN | 0.876 | 0.867 | 0.886 | 0.898 | 0.834 | 0.962 | 0.936 | 0.860 | 1 |
| RSAD2_IFI27_HERC6_MMP8 | 0.876 | 0.867 | 0.885 | 0.896 | 0.837 | 0.956 | 0.893 | 0.813 | 0.974 |
| OAS1_IFI44L_SIGLEC1_FAM20A | 0.876 | 0.866 | 0.886 | 0.893 | 0.820 | 0.965 | 0.955 | 0.895 | 1 |
| IFI44L_HERC6_SLC1A2_OLAH | 0.876 | 0.866 | 0.885 | 0.906 | 0.848 | 0.963 | 0.939 | 0.885 | 0.994 |
| IFIT1_SLC1A2_OLAH_MMP8 | 0.876 | 0.867 | 0.885 | 0.904 | 0.846 | 0.962 | 0.946 | 0.897 | 0.995 |
| IFIT1_IFI44L_IL1R2_FAM20A | 0.876 | 0.867 | 0.885 | 0.896 | 0.831 | 0.962 | 0.949 | 0.883 | 1 |
| RSAD2_IFI27_SLC1A2_RETN | 0.876 | 0.866 | 0.885 | 0.899 | 0.834 | 0.964 | 0.938 | 0.864 | 1 |
| RSAD2_SIGLEC1_IL1R2_RETN | 0.876 | 0.866 | 0.885 | 0.896 | 0.830 | 0.962 | 0.963 | 0.923 | 1 |
| RSAD2_IL1R2_FAM20A_MMP8 | 0.876 | 0.867 | 0.884 | 0.898 | 0.834 | 0.961 | 0.935 | 0.852 | 1 |
| IFI27_OAS1_SLC1A2_RETN | 0.876 | 0.866 | 0.885 | 0.898 | 0.833 | 0.963 | 0.938 | 0.865 | 1 |
| OAS1_SIGLEC1_ISG15_FAM20A | 0.876 | 0.866 | 0.886 | 0.891 | 0.819 | 0.963 | 0.955 | 0.895 | 1 |
| OAS1_SIGLEC1_HERC6_RETN | 0.875 | 0.866 | 0.885 | 0.891 | 0.820 | 0.963 | 0.947 | 0.897 | 0.997 |
| IFI27_IFI44L_HERC6_MMP8 | 0.875 | 0.867 | 0.884 | 0.893 | 0.831 | 0.955 | 0.892 | 0.811 | 0.974 |
| RSAD2_IFI44L_SIGLEC1_FAM20A | 0.875 | 0.866 | 0.885 | 0.893 | 0.822 | 0.964 | 0.950 | 0.889 | 1 |
| RSAD2_IFIT1_IL1R2_FAM20A | 0.875 | 0.866 | 0.884 | 0.898 | 0.832 | 0.964 | 0.948 | 0.877 | 1 |
| IFIT1_SLC1A2_IL1R2_OLAH | 0.875 | 0.867 | 0.884 | 0.908 | 0.851 | 0.965 | 0.945 | 0.894 | 0.995 |
| IFIT1_HERC6_IL1R2_MMP8 | 0.875 | 0.867 | 0.884 | 0.897 | 0.835 | 0.958 | 0.949 | 0.902 | 0.996 |
| IFI27_IFIT1_IFI44L_FAM20A | 0.875 | 0.865 | 0.885 | 0.895 | 0.826 | 0.964 | 0.939 | 0.860 | 1 |
| ISG15_HERC6_FAM20A_RETN | 0.875 | 0.865 | 0.885 | 0.895 | 0.827 | 0.964 | 0.942 | 0.867 | 1 |
| IFI27_IFI44L_SLC1A2_FAM20A | 0.875 | 0.865 | 0.885 | 0.897 | 0.831 | 0.963 | 0.943 | 0.866 | 1 |
| IFI44L_ISG15_HERC6_IL1R2 | 0.875 | 0.867 | 0.883 | 0.896 | 0.838 | 0.955 | 0.943 | 0.892 | 0.995 |
| IFIT1_ISG15_HERC6_IL1R2 | 0.875 | 0.866 | 0.883 | 0.897 | 0.837 | 0.957 | 0.947 | 0.897 | 0.996 |
| ISG15_IL1R2_FAM20A_MMP8 | 0.875 | 0.866 | 0.883 | 0.901 | 0.842 | 0.959 | 0.917 | 0.831 | 1 |
| IFI27_IFIT1_FAM20A_MMP8 | 0.875 | 0.864 | 0.885 | 0.896 | 0.829 | 0.963 | 0.934 | 0.853 | 1 |
| IFI27_OAS1_RETN_MMP8 | 0.875 | 0.865 | 0.885 | 0.900 | 0.836 | 0.964 | 0.940 | 0.878 | 1 |
| IFIT1_IL1R2_OLAH_FAM20A | 0.875 | 0.865 | 0.884 | 0.896 | 0.830 | 0.962 | 0.952 | 0.895 | 1 |
| RSAD2_IFI27_RETN_MMP8 | 0.875 | 0.865 | 0.884 | 0.899 | 0.833 | 0.964 | 0.942 | 0.879 | 1 |
| IFIT1_IFI44L_HERC6_IL1R2 | 0.875 | 0.866 | 0.883 | 0.896 | 0.834 | 0.959 | 0.942 | 0.891 | 0.994 |
| OAS1_SIGLEC1_HERC6_OLAH | 0.875 | 0.865 | 0.884 | 0.893 | 0.828 | 0.957 | 0.953 | 0.909 | 0.998 |
| OAS1_IFIT1_SLC1A2_OLAH | 0.874 | 0.865 | 0.884 | 0.905 | 0.848 | 0.963 | 0.941 | 0.889 | 0.993 |
| IFI44L_IL1R2_FAM20A_RETN | 0.874 | 0.865 | 0.883 | 0.895 | 0.831 | 0.959 | 0.939 | 0.866 | 1 |
| IFIT1_HERC6_IL1R2_RETN | 0.874 | 0.865 | 0.884 | 0.893 | 0.826 | 0.960 | 0.948 | 0.900 | 0.996 |
| IFIT1_IL1R2_FAM20A_RETN | 0.874 | 0.865 | 0.884 | 0.897 | 0.833 | 0.962 | 0.947 | 0.877 | 1 |
| ISG15_HERC6_IL1R2_RETN | 0.874 | 0.865 | 0.884 | 0.896 | 0.832 | 0.960 | 0.948 | 0.898 | 0.997 |
| OAS1_HERC6_SLC1A2_OLAH | 0.874 | 0.865 | 0.884 | 0.902 | 0.844 | 0.960 | 0.936 | 0.879 | 0.993 |
| RSAD2_SIGLEC1_HERC6_OLAH | 0.874 | 0.865 | 0.884 | 0.897 | 0.834 | 0.960 | 0.948 | 0.900 | 0.996 |
| IFIT1_HERC6_SLC1A2_OLAH | 0.874 | 0.865 | 0.884 | 0.903 | 0.845 | 0.961 | 0.941 | 0.887 | 0.996 |
| RSAD2_IFI44L_IL1R2_FAM20A | 0.874 | 0.866 | 0.883 | 0.895 | 0.830 | 0.961 | 0.946 | 0.876 | 1 |
| IFI44L_HERC6_IL1R2_OLAH | 0.874 | 0.865 | 0.883 | 0.899 | 0.839 | 0.958 | 0.948 | 0.899 | 0.996 |
| IFI44L_HERC6_IL1R2_RETN | 0.874 | 0.865 | 0.883 | 0.894 | 0.827 | 0.960 | 0.942 | 0.891 | 0.994 |
| RSAD2_IFIT1_HERC6_IL1R2 | 0.874 | 0.865 | 0.883 | 0.895 | 0.833 | 0.957 | 0.941 | 0.889 | 0.993 |
| IFIT1_HERC6_SLC1A2_IL1R2 | 0.874 | 0.865 | 0.883 | 0.901 | 0.844 | 0.959 | 0.946 | 0.893 | 0.998 |
| RSAD2_HERC6_SLC1A2_OLAH | 0.874 | 0.865 | 0.883 | 0.901 | 0.843 | 0.960 | 0.938 | 0.882 | 0.994 |
| IFI44L_SIGLEC1_OLAH_MMP8 | 0.874 | 0.865 | 0.883 | 0.892 | 0.824 | 0.959 | 0.954 | 0.909 | 0.999 |
| ISG15_SLC1A2_OLAH_FAM20A | 0.874 | 0.865 | 0.883 | 0.901 | 0.842 | 0.961 | 0.912 | 0.830 | 0.994 |
| SIGLEC1_HERC6_OLAH_MMP8 | 0.874 | 0.864 | 0.884 | 0.893 | 0.826 | 0.961 | 0.957 | 0.914 | 0.999 |
| ISG15_HERC6_IL1R2_MMP8 | 0.874 | 0.865 | 0.883 | 0.897 | 0.838 | 0.955 | 0.941 | 0.889 | 0.994 |
| OAS1_IFI44L_SLC1A2_OLAH | 0.874 | 0.864 | 0.884 | 0.907 | 0.850 | 0.964 | 0.934 | 0.878 | 0.990 |
| ISG15_HERC6_IL1R2_OLAH | 0.874 | 0.865 | 0.883 | 0.899 | 0.838 | 0.959 | 0.943 | 0.893 | 0.994 |
| RSAD2_SIGLEC1_HERC6_RETN | 0.874 | 0.864 | 0.883 | 0.891 | 0.820 | 0.962 | 0.936 | 0.882 | 0.990 |
| SIGLEC1_HERC6_OLAH_RETN | 0.874 | 0.864 | 0.884 | 0.893 | 0.821 | 0.964 | 0.957 | 0.914 | 0.999 |
| IFIT1_ISG15_SLC1A2_OLAH | 0.874 | 0.865 | 0.883 | 0.903 | 0.844 | 0.962 | 0.945 | 0.895 | 0.995 |
| IFI27_ISG15_FAM20A_MMP8 | 0.874 | 0.864 | 0.884 | 0.892 | 0.824 | 0.961 | 0.927 | 0.846 | 1 |
| ISG15_HERC6_SLC1A2_OLAH | 0.874 | 0.864 | 0.883 | 0.902 | 0.844 | 0.960 | 0.940 | 0.886 | 0.995 |
| IFI27_IFI44L_SIGLEC1_MMP8 | 0.874 | 0.864 | 0.883 | 0.890 | 0.824 | 0.957 | 0.920 | 0.849 | 0.990 |
| RSAD2_OAS1_OLAH_FAM20A | 0.873 | 0.864 | 0.883 | 0.893 | 0.822 | 0.965 | 0.946 | 0.881 | 1 |
| IFI27_SIGLEC1_SLC1A2_MMP8 | 0.873 | 0.864 | 0.882 | 0.899 | 0.838 | 0.959 | 0.935 | 0.870 | 0.999 |
| RSAD2_ISG15_HERC6_IL1R2 | 0.873 | 0.865 | 0.882 | 0.896 | 0.838 | 0.955 | 0.943 | 0.892 | 0.995 |
| IFIT1_SIGLEC1_OLAH_MMP8 | 0.873 | 0.864 | 0.883 | 0.892 | 0.825 | 0.958 | 0.947 | 0.897 | 0.997 |
| IFI44L_HERC6_IL1R2_MMP8 | 0.873 | 0.865 | 0.882 | 0.897 | 0.837 | 0.956 | 0.940 | 0.887 | 0.993 |
| OAS1_IFIT1_OLAH_FAM20A | 0.873 | 0.863 | 0.883 | 0.891 | 0.819 | 0.963 | 0.949 | 0.886 | 1 |
| RSAD2_IFI44L_SLC1A2_OLAH | 0.873 | 0.864 | 0.882 | 0.906 | 0.849 | 0.964 | 0.934 | 0.878 | 0.990 |
| RSAD2_SLC1A2_IL1R2_OLAH | 0.873 | 0.864 | 0.882 | 0.904 | 0.846 | 0.962 | 0.937 | 0.878 | 0.996 |
| IFIT1_HERC6_IL1R2_OLAH | 0.873 | 0.864 | 0.882 | 0.899 | 0.838 | 0.960 | 0.946 | 0.896 | 0.995 |
| OAS1_ISG15_HERC6_IL1R2 | 0.873 | 0.864 | 0.882 | 0.896 | 0.837 | 0.954 | 0.941 | 0.888 | 0.995 |
| IFI27_OAS1_FAM20A_MMP8 | 0.873 | 0.863 | 0.883 | 0.893 | 0.825 | 0.962 | 0.935 | 0.854 | 1 |
| RSAD2_HERC6_IL1R2_OLAH | 0.873 | 0.864 | 0.882 | 0.898 | 0.838 | 0.959 | 0.945 | 0.894 | 0.995 |
| IFI27_ISG15_HERC6_MMP8 | 0.873 | 0.864 | 0.882 | 0.891 | 0.826 | 0.956 | 0.907 | 0.830 | 0.983 |
| IFI27_IFIT1_SIGLEC1_MMP8 | 0.873 | 0.863 | 0.882 | 0.888 | 0.820 | 0.955 | 0.911 | 0.835 | 0.987 |
| OAS1_HERC6_IL1R2_OLAH | 0.873 | 0.863 | 0.882 | 0.900 | 0.840 | 0.960 | 0.937 | 0.881 | 0.993 |
| ISG15_IL1R2_FAM20A_RETN | 0.873 | 0.864 | 0.882 | 0.900 | 0.838 | 0.961 | 0.925 | 0.842 | 1 |
| RSAD2_OAS1_HERC6_IL1R2 | 0.873 | 0.864 | 0.882 | 0.894 | 0.832 | 0.955 | 0.943 | 0.892 | 0.995 |
| SIGLEC1_OLAH_RETN_MMP8 | 0.873 | 0.863 | 0.883 | 0.892 | 0.821 | 0.962 | 0.964 | 0.925 | 1 |
| HERC6_SLC1A2_IL1R2_OLAH | 0.873 | 0.864 | 0.881 | 0.902 | 0.845 | 0.959 | 0.932 | 0.869 | 0.994 |
| HERC6_SLC1A2_OLAH_MMP8 | 0.872 | 0.863 | 0.882 | 0.902 | 0.844 | 0.960 | 0.936 | 0.878 | 0.993 |
| IFIT1_SLC1A2_OLAH_RETN | 0.872 | 0.863 | 0.882 | 0.906 | 0.847 | 0.965 | 0.948 | 0.899 | 0.996 |
| IFIT1_SLC1A2_IL1R2_MMP8 | 0.872 | 0.864 | 0.881 | 0.904 | 0.847 | 0.960 | 0.953 | 0.908 | 0.999 |
| RSAD2_IFIT1_SIGLEC1_OLAH | 0.872 | 0.863 | 0.882 | 0.894 | 0.827 | 0.962 | 0.947 | 0.896 | 0.998 |
| IFI27_OAS1_IFI44L_FAM20A | 0.872 | 0.862 | 0.882 | 0.894 | 0.825 | 0.964 | 0.942 | 0.863 | 1 |
| OAS1_SLC1A2_IL1R2_OLAH | 0.872 | 0.863 | 0.881 | 0.908 | 0.850 | 0.965 | 0.925 | 0.859 | 0.991 |
| IFI27_OAS1_SIGLEC1_MMP8 | 0.872 | 0.863 | 0.881 | 0.890 | 0.824 | 0.956 | 0.921 | 0.848 | 0.993 |
| RSAD2_SLC1A2_OLAH_MMP8 | 0.872 | 0.863 | 0.881 | 0.901 | 0.842 | 0.959 | 0.936 | 0.880 | 0.992 |
| SIGLEC1_ISG15_HERC6_OLAH | 0.872 | 0.863 | 0.881 | 0.891 | 0.822 | 0.960 | 0.954 | 0.911 | 0.998 |
| ISG15_HERC6_SLC1A2_IL1R2 | 0.872 | 0.863 | 0.881 | 0.901 | 0.846 | 0.957 | 0.936 | 0.871 | 1 |
| IFIT1_ISG15_SLC1A2_IL1R2 | 0.872 | 0.864 | 0.880 | 0.903 | 0.845 | 0.960 | 0.948 | 0.898 | 0.998 |
| OAS1_SLC1A2_OLAH_MMP8 | 0.872 | 0.863 | 0.881 | 0.902 | 0.844 | 0.960 | 0.932 | 0.872 | 0.991 |
| OAS1_HERC6_IL1R2_RETN | 0.872 | 0.863 | 0.881 | 0.894 | 0.828 | 0.959 | 0.939 | 0.885 | 0.993 |
| RSAD2_IFI27_FAM20A_MMP8 | 0.872 | 0.862 | 0.882 | 0.894 | 0.826 | 0.962 | 0.934 | 0.853 | 1 |
| OAS1_HERC6_IL1R2_MMP8 | 0.872 | 0.863 | 0.881 | 0.893 | 0.831 | 0.955 | 0.932 | 0.873 | 0.990 |
| RSAD2_IL1R2_FAM20A_RETN | 0.872 | 0.863 | 0.881 | 0.893 | 0.826 | 0.961 | 0.943 | 0.868 | 1 |
| IFI27_SIGLEC1_HERC6_MMP8 | 0.872 | 0.863 | 0.881 | 0.889 | 0.823 | 0.956 | 0.918 | 0.846 | 0.991 |
| IFI27_OAS1_SIGLEC1_SLC1A2 | 0.872 | 0.863 | 0.881 | 0.896 | 0.837 | 0.956 | 0.957 | 0.908 | 1 |
| HERC6_SLC1A2_OLAH_RETN | 0.872 | 0.862 | 0.881 | 0.903 | 0.844 | 0.962 | 0.935 | 0.874 | 0.995 |
| ISG15_SLC1A2_IL1R2_OLAH | 0.872 | 0.863 | 0.880 | 0.897 | 0.839 | 0.956 | 0.921 | 0.847 | 0.994 |
| RSAD2_IFI27_SIGLEC1_HERC6 | 0.872 | 0.863 | 0.881 | 0.896 | 0.840 | 0.951 | 0.922 | 0.859 | 0.985 |
| RSAD2_OAS1_SIGLEC1_OLAH | 0.872 | 0.862 | 0.881 | 0.891 | 0.822 | 0.960 | 0.945 | 0.893 | 0.996 |
| IFI44L_HERC6_SLC1A2_IL1R2 | 0.872 | 0.863 | 0.881 | 0.900 | 0.844 | 0.956 | 0.935 | 0.874 | 0.996 |
| IFI44L_ISG15_SLC1A2_IL1R2 | 0.872 | 0.864 | 0.880 | 0.900 | 0.844 | 0.955 | 0.937 | 0.874 | 1.000 |
| IFIT1_IFI44L_SIGLEC1_OLAH | 0.872 | 0.863 | 0.881 | 0.893 | 0.826 | 0.960 | 0.938 | 0.881 | 0.995 |
| OAS1_IFI44L_HERC6_IL1R2 | 0.872 | 0.863 | 0.880 | 0.895 | 0.833 | 0.956 | 0.941 | 0.889 | 0.994 |
| RSAD2_IFIT1_SLC1A2_OLAH | 0.872 | 0.862 | 0.881 | 0.902 | 0.844 | 0.961 | 0.945 | 0.895 | 0.995 |
| OAS1_OLAH_FAM20A_MMP8 | 0.872 | 0.862 | 0.881 | 0.894 | 0.824 | 0.965 | 0.950 | 0.886 | 1 |
| IFI27_OAS1_SIGLEC1_HERC6 | 0.872 | 0.862 | 0.881 | 0.896 | 0.841 | 0.952 | 0.937 | 0.883 | 0.991 |
| IFI27_SLC1A2_RETN_MMP8 | 0.872 | 0.862 | 0.881 | 0.897 | 0.834 | 0.961 | 0.914 | 0.827 | 1 |
| IFIT1_SIGLEC1_ISG15_OLAH | 0.872 | 0.863 | 0.880 | 0.894 | 0.827 | 0.961 | 0.946 | 0.893 | 0.998 |
| RSAD2_OAS1_SLC1A2_OLAH | 0.871 | 0.862 | 0.881 | 0.902 | 0.844 | 0.961 | 0.934 | 0.876 | 0.991 |
| HERC6_IL1R2_OLAH_RETN | 0.871 | 0.862 | 0.881 | 0.896 | 0.832 | 0.961 | 0.940 | 0.887 | 0.993 |
| IFIT1_SIGLEC1_OLAH_RETN | 0.871 | 0.862 | 0.881 | 0.892 | 0.823 | 0.962 | 0.941 | 0.884 | 0.998 |
| OAS1_SIGLEC1_OLAH_MMP8 | 0.871 | 0.862 | 0.881 | 0.890 | 0.821 | 0.958 | 0.954 | 0.910 | 0.999 |
| OAS1_IFIT1_SLC1A2_IL1R2 | 0.871 | 0.863 | 0.880 | 0.903 | 0.845 | 0.961 | 0.946 | 0.895 | 0.997 |
| RSAD2_HERC6_IL1R2_RETN | 0.871 | 0.862 | 0.881 | 0.894 | 0.828 | 0.960 | 0.941 | 0.889 | 0.994 |
| HERC6_IL1R2_OLAH_MMP8 | 0.871 | 0.862 | 0.880 | 0.897 | 0.838 | 0.956 | 0.935 | 0.877 | 0.992 |
| IFI44L_SIGLEC1_OLAH_RETN | 0.871 | 0.862 | 0.881 | 0.891 | 0.821 | 0.961 | 0.943 | 0.888 | 0.998 |
| RSAD2_IFI44L_HERC6_IL1R2 | 0.871 | 0.862 | 0.880 | 0.894 | 0.833 | 0.955 | 0.942 | 0.891 | 0.994 |
| HERC6_IL1R2_RETN_MMP8 | 0.871 | 0.862 | 0.881 | 0.893 | 0.828 | 0.958 | 0.930 | 0.867 | 0.994 |
| RSAD2_HERC6_IL1R2_MMP8 | 0.871 | 0.862 | 0.880 | 0.895 | 0.833 | 0.956 | 0.943 | 0.892 | 0.995 |
| OAS1_HERC6_SLC1A2_IL1R2 | 0.871 | 0.862 | 0.880 | 0.900 | 0.844 | 0.957 | 0.926 | 0.859 | 0.994 |
| RSAD2_SLC1A2_OLAH_RETN | 0.871 | 0.862 | 0.880 | 0.902 | 0.843 | 0.961 | 0.940 | 0.886 | 0.994 |
| IFI44L_SIGLEC1_ISG15_OLAH | 0.871 | 0.862 | 0.880 | 0.891 | 0.822 | 0.959 | 0.947 | 0.896 | 0.998 |
| SIGLEC1_ISG15_OLAH_MMP8 | 0.871 | 0.861 | 0.880 | 0.890 | 0.822 | 0.958 | 0.953 | 0.908 | 0.999 |
| RSAD2_HERC6_SLC1A2_IL1R2 | 0.871 | 0.862 | 0.880 | 0.899 | 0.842 | 0.956 | 0.932 | 0.865 | 0.998 |
| IFI44L_HERC6_FAM20A_RETN | 0.871 | 0.861 | 0.881 | 0.892 | 0.821 | 0.963 | 0.941 | 0.864 | 1 |
| IFI27_HERC6_SLC1A2_MMP8 | 0.871 | 0.861 | 0.880 | 0.900 | 0.843 | 0.957 | 0.904 | 0.824 | 0.985 |
| OAS1_IFI44L_SIGLEC1_OLAH | 0.871 | 0.861 | 0.880 | 0.892 | 0.824 | 0.960 | 0.948 | 0.899 | 0.997 |
| OAS1_ISG15_OLAH_FAM20A | 0.871 | 0.861 | 0.881 | 0.891 | 0.819 | 0.963 | 0.951 | 0.891 | 1 |
| IFI44L_ISG15_OLAH_FAM20A | 0.871 | 0.861 | 0.880 | 0.890 | 0.818 | 0.961 | 0.958 | 0.912 | 1 |
| SIGLEC1_ISG15_OLAH_RETN | 0.871 | 0.861 | 0.880 | 0.891 | 0.820 | 0.962 | 0.949 | 0.897 | 1 |
| IFI27_IFIT1_ISG15_FAM20A | 0.870 | 0.860 | 0.881 | 0.887 | 0.816 | 0.958 | 0.942 | 0.864 | 1 |
| IFIT1_IFI44L_SLC1A2_IL1R2 | 0.870 | 0.862 | 0.879 | 0.900 | 0.841 | 0.959 | 0.948 | 0.898 | 0.997 |
| OAS1_IFI44L_OLAH_FAM20A | 0.870 | 0.860 | 0.880 | 0.890 | 0.817 | 0.963 | 0.953 | 0.898 | 1 |
| OAS1_ISG15_SLC1A2_OLAH | 0.870 | 0.861 | 0.880 | 0.906 | 0.849 | 0.963 | 0.930 | 0.869 | 0.992 |
| RSAD2_SIGLEC1_OLAH_MMP8 | 0.870 | 0.861 | 0.880 | 0.890 | 0.822 | 0.958 | 0.953 | 0.908 | 0.999 |
| OAS1_OLAH_FAM20A_RETN | 0.870 | 0.860 | 0.880 | 0.893 | 0.822 | 0.965 | 0.950 | 0.888 | 1 |
| IFI44L_SLC1A2_IL1R2_RETN | 0.870 | 0.861 | 0.879 | 0.899 | 0.842 | 0.956 | 0.938 | 0.879 | 0.997 |
| IFI44L_OLAH_FAM20A_MMP8 | 0.870 | 0.860 | 0.880 | 0.888 | 0.817 | 0.959 | 0.958 | 0.911 | 1 |
| OAS1_SIGLEC1_OLAH_RETN | 0.870 | 0.860 | 0.880 | 0.890 | 0.819 | 0.961 | 0.949 | 0.898 | 1 |
| IFIT1_SLC1A2_IL1R2_RETN | 0.870 | 0.861 | 0.879 | 0.899 | 0.839 | 0.959 | 0.950 | 0.902 | 0.998 |
| OAS1_SLC1A2_OLAH_RETN | 0.870 | 0.861 | 0.879 | 0.903 | 0.845 | 0.961 | 0.932 | 0.871 | 0.992 |
| RSAD2_IFI27_SIGLEC1_MMP8 | 0.870 | 0.860 | 0.880 | 0.888 | 0.821 | 0.955 | 0.915 | 0.842 | 0.988 |
| RSAD2_IFIT1_SLC1A2_IL1R2 | 0.870 | 0.862 | 0.878 | 0.899 | 0.840 | 0.958 | 0.949 | 0.899 | 0.999 |
| RSAD2_ISG15_SLC1A2_OLAH | 0.870 | 0.861 | 0.879 | 0.901 | 0.843 | 0.960 | 0.935 | 0.878 | 0.991 |
| ISG15_HERC6_OLAH_RETN | 0.870 | 0.860 | 0.880 | 0.892 | 0.822 | 0.962 | 0.953 | 0.909 | 0.998 |
| IFI44L_SIGLEC1_HERC6_RETN | 0.869 | 0.860 | 0.879 | 0.883 | 0.808 | 0.959 | 0.943 | 0.894 | 0.993 |
| IFI44L_SLC1A2_IL1R2_MMP8 | 0.869 | 0.861 | 0.877 | 0.897 | 0.842 | 0.953 | 0.936 | 0.871 | 1 |
| RSAD2_SIGLEC1_OLAH_RETN | 0.869 | 0.860 | 0.879 | 0.890 | 0.819 | 0.961 | 0.948 | 0.896 | 1.000 |
| OAS1_IFIT1_IL1R2_OLAH | 0.869 | 0.860 | 0.879 | 0.893 | 0.826 | 0.961 | 0.946 | 0.896 | 0.995 |
| HERC6_SLC1A2_IL1R2_RETN | 0.869 | 0.860 | 0.879 | 0.899 | 0.841 | 0.958 | 0.915 | 0.839 | 0.992 |
| IFI27_ISG15_SLC1A2_FAM20A | 0.869 | 0.859 | 0.879 | 0.888 | 0.817 | 0.960 | 0.926 | 0.843 | 1 |
| IFIT1_HERC6_FAM20A_RETN | 0.869 | 0.859 | 0.879 | 0.887 | 0.816 | 0.958 | 0.937 | 0.859 | 1 |
| IFI27_IFI44L_SLC1A2_MMP8 | 0.869 | 0.860 | 0.879 | 0.893 | 0.831 | 0.955 | 0.912 | 0.837 | 0.987 |
| OAS1_IFIT1_IL1R2_MMP8 | 0.869 | 0.860 | 0.878 | 0.892 | 0.828 | 0.956 | 0.954 | 0.911 | 0.998 |
| HERC6_SLC1A2_FAM20A_RETN | 0.869 | 0.859 | 0.879 | 0.896 | 0.833 | 0.960 | 0.934 | 0.851 | 1 |
| HERC6_OLAH_RETN_MMP8 | 0.869 | 0.859 | 0.879 | 0.892 | 0.822 | 0.962 | 0.957 | 0.915 | 0.998 |
| IFI27_IFIT1_SIGLEC1_SLC1A2 | 0.869 | 0.860 | 0.878 | 0.894 | 0.834 | 0.954 | 0.954 | 0.907 | 1 |
| HERC6_SLC1A2_IL1R2_MMP8 | 0.869 | 0.860 | 0.878 | 0.900 | 0.845 | 0.955 | 0.910 | 0.828 | 0.991 |
| RSAD2_IFI27_OAS1_FAM20A | 0.869 | 0.859 | 0.879 | 0.884 | 0.811 | 0.957 | 0.938 | 0.858 | 1 |
| IFI27_SIGLEC1_ISG15_MMP8 | 0.869 | 0.859 | 0.878 | 0.884 | 0.814 | 0.954 | 0.917 | 0.845 | 0.989 |
| OAS1_HERC6_OLAH_RETN | 0.869 | 0.859 | 0.878 | 0.888 | 0.819 | 0.958 | 0.942 | 0.891 | 0.993 |
| IFI44L_HERC6_OLAH_RETN | 0.869 | 0.859 | 0.879 | 0.889 | 0.817 | 0.960 | 0.945 | 0.895 | 0.94 |
| IFIT1_HERC6_OLAH_RETN | 0.869 | 0.859 | 0.878 | 0.889 | 0.820 | 0.959 | 0.940 | 0.888 | 0.993 |
| IFI27_OAS1_ISG15_FAM20A | 0.869 | 0.858 | 0.879 | 0.883 | 0.810 | 0.956 | 0.942 | 0.864 | 1 |
| IFIT1_IFI44L_IL1R2_MMP8 | 0.868 | 0.859 | 0.877 | 0.892 | 0.828 | 0.955 | 0.957 | 0.914 | 0.999 |
| OAS1_IFIT1_IFI44L_IL1R2 | 0.868 | 0.859 | 0.877 | 0.888 | 0.820 | 0.957 | 0.945 | 0.895 | 0.994 |
| RSAD2_HERC6_OLAH_RETN | 0.868 | 0.858 | 0.878 | 0.888 | 0.818 | 0.958 | 0.941 | 0.890 | 0.993 |
| IFIT1_IFI44L_ISG15_IL1R2 | 0.868 | 0.859 | 0.877 | 0.894 | 0.831 | 0.958 | 0.957 | 0.914 | 0.999 |
| OAS1_SIGLEC1_ISG15_OLAH | 0.868 | 0.858 | 0.878 | 0.889 | 0.819 | 0.958 | 0.945 | 0.892 | 0.997 |
| SIGLEC1_HERC6_SLC1A2_RETN | 0.868 | 0.858 | 0.877 | 0.893 | 0.827 | 0.960 | 0.955 | 0.910 | 1 |
| IFIT1_IL1R2_OLAH_MMP8 | 0.868 | 0.858 | 0.877 | 0.890 | 0.826 | 0.954 | 0.961 | 0.922 | 1 |
| OAS1_ISG15_HERC6_RETN | 0.868 | 0.858 | 0.878 | 0.890 | 0.821 | 0.959 | 0.941 | 0.886 | 0.996 |
| ISG15_SLC1A2_OLAH_MMP8 | 0.868 | 0.858 | 0.877 | 0.894 | 0.835 | 0.954 | 0.922 | 0.856 | 0.988 |
| ISG15_SLC1A2_OLAH_RETN | 0.868 | 0.858 | 0.877 | 0.897 | 0.838 | 0.956 | 0.924 | 0.856 | 0.992 |
| RSAD2_IFI27_SIGLEC1_SLC1A2 | 0.868 | 0.858 | 0.877 | 0.895 | 0.836 | 0.955 | 0.951 | 0.902 | 1 |
| OAS1_IFIT1_ISG15_IL1R2 | 0.868 | 0.859 | 0.877 | 0.891 | 0.825 | 0.957 | 0.949 | 0.902 | 0.996 |
| OAS1_HERC6_FAM20A_RETN | 0.868 | 0.858 | 0.878 | 0.887 | 0.816 | 0.959 | 0.939 | 0.861 | 1 |
| IFI27_SLC1A2_FAM20A_MMP8 | 0.868 | 0.857 | 0.878 | 0.891 | 0.825 | 0.958 | 0.907 | 0.815 | 0.998 |
| IFI27_OAS1_IFIT1_FAM20A | 0.868 | 0.857 | 0.878 | 0.885 | 0.813 | 0.956 | 0.943 | 0.865 | 1 |
| IFIT1_ISG15_IL1R2_MMP8 | 0.868 | 0.859 | 0.876 | 0.893 | 0.831 | 0.955 | 0.963 | 0.925 | 1 |
| RSAD2_OAS1_IFIT1_IL1R2 | 0.868 | 0.859 | 0.877 | 0.888 | 0.820 | 0.956 | 0.947 | 0.899 | 0.995 |
| RSAD2_OAS1_IL1R2_OLAH | 0.867 | 0.858 | 0.877 | 0.893 | 0.827 | 0.960 | 0.945 | 0.891 | 0.998 |
| IFI27_IFI44L_SIGLEC1_HERC6 | 0.867 | 0.858 | 0.877 | 0.891 | 0.830 | 0.952 | 0.932 | 0.875 | 0.988 |
| RSAD2_IFI44L_SIGLEC1_OLAH | 0.867 | 0.858 | 0.877 | 0.891 | 0.822 | 0.960 | 0.952 | 0.905 | 0.999 |
| IFI27_IFIT1_SLC1A2_FAM20A | 0.867 | 0.857 | 0.878 | 0.890 | 0.821 | 0.958 | 0.941 | 0.862 | 1 |
| RSAD2_IFI27_IFIT1_FAM20A | 0.867 | 0.857 | 0.878 | 0.888 | 0.817 | 0.958 | 0.940 | 0.861 | 1 |
| IFI27_IFI44L_SIGLEC1_SLC1A2 | 0.867 | 0.858 | 0.876 | 0.893 | 0.831 | 0.954 | 0.960 | 0.914 | 1 |
| RSAD2_SIGLEC1_ISG15_OLAH | 0.867 | 0.858 | 0.876 | 0.890 | 0.821 | 0.959 | 0.950 | 0.902 | 0.998 |
| IFI27_SIGLEC1_ISG15_SLC1A2 | 0.867 | 0.858 | 0.876 | 0.892 | 0.831 | 0.952 | 0.959 | 0.912 | 1 |
| RSAD2_HERC6_FAM20A_RETN | 0.867 | 0.857 | 0.877 | 0.886 | 0.815 | 0.958 | 0.938 | 0.860 | 1 |
| IFI44L_ISG15_IL1R2_OLAH | 0.867 | 0.858 | 0.876 | 0.892 | 0.830 | 0.954 | 0.948 | 0.899 | 0.996 |
| RSAD2_IFI27_ISG15_FAM20A | 0.867 | 0.857 | 0.877 | 0.885 | 0.814 | 0.957 | 0.945 | 0.866 | 1 |
| RSAD2_IFIT1_IL1R2_MMP8 | 0.867 | 0.858 | 0.876 | 0.891 | 0.827 | 0.955 | 0.960 | 0.920 | 1.000 |
| IFIT1_IFI44L_OLAH_FAM20A | 0.867 | 0.857 | 0.877 | 0.889 | 0.818 | 0.960 | 0.957 | 0.909 | 1 |
| IFIT1_HERC6_OLAH_MMP8 | 0.867 | 0.857 | 0.876 | 0.887 | 0.819 | 0.954 | 0.940 | 0.887 | 0.993 |
| IFIT1_ISG15_IL1R2_OLAH | 0.867 | 0.857 | 0.876 | 0.895 | 0.830 | 0.959 | 0.959 | 0.918 | 1.000 |
| SIGLEC1_ISG15_HERC6_RETN | 0.866 | 0.856 | 0.877 | 0.879 | 0.801 | 0.958 | 0.959 | 0.916 | 1 |
| OAS1_IFIT1_IL1R2_RETN | 0.866 | 0.857 | 0.876 | 0.888 | 0.817 | 0.958 | 0.950 | 0.904 | 0.996 |
| IFI44L_HERC6_OLAH_MMP8 | 0.866 | 0.857 | 0.876 | 0.887 | 0.819 | 0.955 | 0.941 | 0.889 | 0.993 |
| IFI44L_OLAH_FAM20A_RETN | 0.866 | 0.856 | 0.876 | 0.889 | 0.818 | 0.960 | 0.957 | 0.909 | 1 |
| IFIT1_ISG15_IL1R2_RETN | 0.866 | 0.857 | 0.876 | 0.890 | 0.824 | 0.957 | 0.955 | 0.912 | 0.999 |
| IFI27_OAS1_SLC1A2_FAM20A | 0.866 | 0.856 | 0.877 | 0.888 | 0.820 | 0.957 | 0.938 | 0.857 | 1 |
| OAS1_IFI44L_IL1R2_OLAH | 0.866 | 0.857 | 0.876 | 0.893 | 0.827 | 0.959 | 0.940 | 0.885 | 0.995 |
| OAS1_SIGLEC1_SLC1A2_RETN | 0.866 | 0.857 | 0.875 | 0.893 | 0.829 | 0.957 | 0.951 | 0.903 | 0.999 |
| OAS1_IL1R2_OLAH_MMP8 | 0.866 | 0.857 | 0.875 | 0.894 | 0.831 | 0.957 | 0.939 | 0.883 | 0.996 |
| RSAD2_OAS1_HERC6_OLAH | 0.866 | 0.856 | 0.875 | 0.887 | 0.821 | 0.953 | 0.937 | 0.882 | 0.992 |
| IFI44L_IL1R2_OLAH_MMP8 | 0.866 | 0.857 | 0.875 | 0.892 | 0.832 | 0.951 | 0.946 | 0.895 | 0.996 |
| OAS1_ISG15_IL1R2_OLAH | 0.866 | 0.857 | 0.875 | 0.893 | 0.826 | 0.959 | 0.942 | 0.888 | 0.997 |
| IFIT1_SIGLEC1_SLC1A2_RETN | 0.866 | 0.857 | 0.875 | 0.894 | 0.832 | 0.956 | 0.947 | 0.897 | 0.996 |
| IFIT1_IL1R2_RETN_MMP8 | 0.866 | 0.856 | 0.876 | 0.890 | 0.820 | 0.959 | 0.965 | 0.929 | 1 |
| ISG15_SLC1A2_IL1R2_RETN | 0.866 | 0.857 | 0.874 | 0.898 | 0.840 | 0.956 | 0.915 | 0.834 | 0.996 |
| ISG15_HERC6_OLAH_MMP8 | 0.866 | 0.856 | 0.875 | 0.888 | 0.820 | 0.955 | 0.946 | 0.897 | 0.994 |
| RSAD2_IFI27_SLC1A2_FAM20A | 0.865 | 0.855 | 0.876 | 0.886 | 0.817 | 0.956 | 0.939 | 0.858 | 1 |
| HERC6_FAM20A_RETN_MMP8 | 0.865 | 0.855 | 0.876 | 0.888 | 0.817 | 0.959 | 0.941 | 0.864 | 1 |
| OAS1_HERC6_OLAH_MMP8 | 0.865 | 0.856 | 0.875 | 0.887 | 0.820 | 0.954 | 0.940 | 0.887 | 0.993 |
| RSAD2_ISG15_SLC1A2_IL1R2 | 0.865 | 0.857 | 0.873 | 0.896 | 0.838 | 0.954 | 0.939 | 0.877 | 1 |
| RSAD2_IFIT1_ISG15_IL1R2 | 0.865 | 0.856 | 0.874 | 0.891 | 0.826 | 0.956 | 0.955 | 0.913 | 0.998 |
| IFI27_IFIT1_SLC1A2_MMP8 | 0.865 | 0.855 | 0.875 | 0.889 | 0.826 | 0.953 | 0.920 | 0.847 | 0.992 |
| RSAD2_IFI27_SLC1A2_MMP8 | 0.865 | 0.855 | 0.874 | 0.888 | 0.824 | 0.952 | 0.911 | 0.832 | 0.990 |
| OAS1_IL1R2_OLAH_RETN | 0.865 | 0.855 | 0.874 | 0.895 | 0.827 | 0.962 | 0.942 | 0.888 | 0.997 |
| IFI44L_ISG15_IL1R2_MMP8 | 0.865 | 0.857 | 0.873 | 0.892 | 0.834 | 0.950 | 0.954 | 0.909 | 1 |
| IFIT1_ISG15_OLAH_FAM20A | 0.865 | 0.855 | 0.875 | 0.888 | 0.816 | 0.960 | 0.955 | 0.906 | 1 |
| RSAD2_OLAH_FAM20A_MMP8 | 0.865 | 0.855 | 0.875 | 0.886 | 0.814 | 0.958 | 0.959 | 0.907 | 1 |
| IFI44L_ISG15_HERC6_OLAH | 0.865 | 0.855 | 0.874 | 0.885 | 0.818 | 0.952 | 0.950 | 0.904 | 0.996 |
| IFIT1_IFI44L_HERC6_OLAH | 0.865 | 0.855 | 0.874 | 0.887 | 0.820 | 0.953 | 0.939 | 0.885 | 0.993 |
| IFIT1_IFI44L_IL1R2_OLAH | 0.865 | 0.855 | 0.874 | 0.891 | 0.826 | 0.956 | 0.953 | 0.908 | 0.998 |
| OAS1_ISG15_HERC6_OLAH | 0.865 | 0.855 | 0.874 | 0.885 | 0.819 | 0.950 | 0.951 | 0.905 | 0.997 |
| SIGLEC1_ISG15_SLC1A2_RETN | 0.865 | 0.856 | 0.874 | 0.893 | 0.830 | 0.957 | 0.951 | 0.903 | 0.999 |
| RSAD2_IFIT1_IFI44L_IL1R2 | 0.865 | 0.855 | 0.874 | 0.887 | 0.819 | 0.955 | 0.955 | 0.913 | 0.998 |
| RSAD2_HERC6_OLAH_MMP8 | 0.864 | 0.855 | 0.874 | 0.888 | 0.822 | 0.954 | 0.939 | 0.885 | 0.993 |
| IFIT1_ISG15_HERC6_OLAH | 0.864 | 0.855 | 0.874 | 0.885 | 0.818 | 0.951 | 0.948 | 0.900 | 0.995 |
| RSAD2_ISG15_HERC6_RETN | 0.864 | 0.854 | 0.875 | 0.890 | 0.821 | 0.958 | 0.938 | 0.883 | 0.993 |
| IFI44L_ISG15_IL1R2_RETN | 0.864 | 0.855 | 0.873 | 0.888 | 0.823 | 0.953 | 0.959 | 0.917 | 1 |
| OAS1_IFIT1_HERC6_OLAH | 0.864 | 0.855 | 0.874 | 0.886 | 0.819 | 0.953 | 0.941 | 0.889 | 0.993 |
| IFI44L_SIGLEC1_SLC1A2_RETN | 0.864 | 0.856 | 0.873 | 0.894 | 0.830 | 0.958 | 0.951 | 0.903 | 0.999 |
| OAS1_ISG15_SLC1A2_IL1R2 | 0.864 | 0.856 | 0.873 | 0.897 | 0.840 | 0.955 | 0.922 | 0.852 | 0.992 |
| RSAD2_IFI44L_SLC1A2_IL1R2 | 0.864 | 0.856 | 0.873 | 0.896 | 0.838 | 0.954 | 0.941 | 0.883 | 1.000 |
| RSAD2_IFI44L_OLAH_FAM20A | 0.864 | 0.854 | 0.875 | 0.888 | 0.816 | 0.960 | 0.955 | 0.906 | 1 |
| RSAD2_OAS1_SLC1A2_IL1R2 | 0.864 | 0.856 | 0.873 | 0.896 | 0.838 | 0.954 | 0.936 | 0.873 | 0.999 |
| RSAD2_SLC1A2_IL1R2_MMP8 | 0.864 | 0.856 | 0.873 | 0.896 | 0.838 | 0.954 | 0.938 | 0.874 | 1 |
| RSAD2_SLC1A2_IL1R2_RETN | 0.864 | 0.855 | 0.873 | 0.897 | 0.838 | 0.956 | 0.938 | 0.874 | 1 |
| OAS1_SIGLEC1_HERC6_SLC1A2 | 0.864 | 0.855 | 0.873 | 0.886 | 0.824 | 0.949 | 0.947 | 0.898 | 0.996 |
| RSAD2_SIGLEC1_SLC1A2_RETN | 0.864 | 0.855 | 0.873 | 0.893 | 0.830 | 0.957 | 0.951 | 0.904 | 0.998 |
| OAS1_IFI44L_SLC1A2_IL1R2 | 0.864 | 0.855 | 0.873 | 0.898 | 0.841 | 0.954 | 0.934 | 0.873 | 0.994 |
| SIGLEC1_HERC6_RETN_MMP8 | 0.864 | 0.854 | 0.874 | 0.879 | 0.799 | 0.958 | 0.955 | 0.911 | 0.999 |
| RSAD2_ISG15_HERC6_OLAH | 0.864 | 0.854 | 0.874 | 0.885 | 0.820 | 0.950 | 0.946 | 0.897 | 0.994 |
| RSAD2_IFIT1_IL1R2_OLAH | 0.864 | 0.855 | 0.873 | 0.891 | 0.825 | 0.957 | 0.951 | 0.905 | 0.997 |
| RSAD2_IL1R2_OLAH_MMP8 | 0.864 | 0.855 | 0.873 | 0.891 | 0.828 | 0.954 | 0.957 | 0.914 | 0.999 |
| IFI27_IFIT1_SIGLEC1_HERC6 | 0.864 | 0.854 | 0.873 | 0.890 | 0.831 | 0.949 | 0.927 | 0.866 | 0.989 |
| OAS1_IFIT1_SIGLEC1_RETN | 0.864 | 0.854 | 0.874 | 0.878 | 0.803 | 0.953 | 0.940 | 0.887 | 0.994 |
| IFI27_ISG15_SLC1A2_MMP8 | 0.864 | 0.854 | 0.873 | 0.886 | 0.821 | 0.951 | 0.903 | 0.820 | 0.986 |
| OAS1_IFI44L_HERC6_OLAH | 0.864 | 0.854 | 0.873 | 0.886 | 0.820 | 0.953 | 0.941 | 0.889 | 0.993 |
| IFI27_OAS1_SLC1A2_MMP8 | 0.863 | 0.854 | 0.873 | 0.887 | 0.823 | 0.951 | 0.908 | 0.828 | 0.987 |
| IFIT1_IFI44L_IL1R2_RETN | 0.863 | 0.854 | 0.873 | 0.886 | 0.817 | 0.956 | 0.957 | 0.914 | 0.999 |
| IFIT1_SIGLEC1_HERC6_MMP8 | 0.863 | 0.854 | 0.873 | 0.881 | 0.813 | 0.950 | 0.887 | 0.805 | 0.969 |
| RSAD2_SIGLEC1_HERC6_SLC1A2 | 0.863 | 0.855 | 0.872 | 0.887 | 0.824 | 0.949 | 0.933 | 0.876 | 0.989 |
| RSAD2_IFIT1_IL1R2_RETN | 0.863 | 0.854 | 0.873 | 0.884 | 0.813 | 0.955 | 0.955 | 0.913 | 0.998 |
| IFI27_SIGLEC1_HERC6_SLC1A2 | 0.863 | 0.854 | 0.873 | 0.890 | 0.828 | 0.952 | 0.965 | 0.924 | 1 |
| RSAD2_ISG15_OLAH_FAM20A | 0.863 | 0.853 | 0.873 | 0.885 | 0.812 | 0.958 | 0.959 | 0.907 | 1 |
| RSAD2_ISG15_IL1R2_OLAH | 0.863 | 0.854 | 0.872 | 0.890 | 0.824 | 0.956 | 0.953 | 0.908 | 0.999 |
| RSAD2_OAS1_IFI44L_IL1R2 | 0.863 | 0.854 | 0.872 | 0.884 | 0.818 | 0.951 | 0.937 | 0.882 | 0.992 |
| RSAD2_OLAH_FAM20A_RETN | 0.863 | 0.853 | 0.873 | 0.886 | 0.813 | 0.959 | 0.958 | 0.904 | 1 |
| RSAD2_IFI44L_IL1R2_OLAH | 0.863 | 0.854 | 0.872 | 0.891 | 0.826 | 0.955 | 0.946 | 0.895 | 0.997 |
| ISG15_SLC1A2_IL1R2_MMP8 | 0.863 | 0.855 | 0.871 | 0.894 | 0.837 | 0.951 | 0.917 | 0.837 | 0.998 |
| RSAD2_IFI44L_HERC6_OLAH | 0.863 | 0.853 | 0.872 | 0.887 | 0.821 | 0.953 | 0.938 | 0.884 | 0.992 |
| ISG15_IL1R2_OLAH_MMP8 | 0.863 | 0.854 | 0.872 | 0.889 | 0.830 | 0.949 | 0.938 | 0.877 | 0.999 |
| SIGLEC1_HERC6_SLC1A2_MMP8 | 0.863 | 0.854 | 0.872 | 0.887 | 0.822 | 0.952 | 0.934 | 0.877 | 0.991 |
| OAS1_SLC1A2_IL1R2_MMP8 | 0.863 | 0.854 | 0.871 | 0.897 | 0.839 | 0.955 | 0.918 | 0.846 | 0.991 |
| RSAD2_IFIT1_OLAH_FAM20A | 0.863 | 0.853 | 0.873 | 0.886 | 0.812 | 0.959 | 0.957 | 0.904 | 1 |
| HERC6_SLC1A2_FAM20A_MMP8 | 0.863 | 0.853 | 0.873 | 0.888 | 0.824 | 0.952 | 0.922 | 0.836 | 1 |
| OAS1_SLC1A2_IL1R2_RETN | 0.862 | 0.853 | 0.871 | 0.893 | 0.834 | 0.953 | 0.918 | 0.847 | 0.990 |
| OAS1_IFI44L_ISG15_IL1R2 | 0.862 | 0.853 | 0.871 | 0.887 | 0.822 | 0.952 | 0.941 | 0.889 | 0.993 |
| RSAD2_IL1R2_OLAH_RETN | 0.862 | 0.853 | 0.872 | 0.887 | 0.819 | 0.956 | 0.951 | 0.904 | 0.998 |
| IFI44L_SLC1A2_FAM20A_RETN | 0.862 | 0.852 | 0.872 | 0.890 | 0.824 | 0.956 | 0.927 | 0.848 | 1 |
| IFIT1_IL1R2_OLAH_RETN | 0.862 | 0.853 | 0.872 | 0.888 | 0.819 | 0.956 | 0.955 | 0.912 | 0.999 |
| RSAD2_IFIT1_HERC6_OLAH | 0.862 | 0.853 | 0.872 | 0.886 | 0.819 | 0.952 | 0.939 | 0.886 | 0.992 |
| ISG15_IL1R2_OLAH_RETN | 0.862 | 0.853 | 0.871 | 0.889 | 0.826 | 0.953 | 0.940 | 0.878 | 1 |
| IFIT1_OLAH_FAM20A_MMP8 | 0.862 | 0.852 | 0.872 | 0.882 | 0.810 | 0.955 | 0.958 | 0.908 | 1 |
| OAS1_IFI44L_IL1R2_MMP8 | 0.862 | 0.853 | 0.871 | 0.886 | 0.822 | 0.951 | 0.938 | 0.883 | 0.993 |
| IFIT1_SIGLEC1_HERC6_SLC1A2 | 0.862 | 0.853 | 0.871 | 0.887 | 0.825 | 0.948 | 0.935 | 0.879 | 0.990 |
| IFI44L_SIGLEC1_SLC1A2_MMP8 | 0.862 | 0.853 | 0.870 | 0.884 | 0.819 | 0.949 | 0.937 | 0.882 | 0.992 |
| IFI27_IFI44L_SIGLEC1_ISG15 | 0.862 | 0.852 | 0.871 | 0.886 | 0.822 | 0.949 | 0.941 | 0.887 | 0.996 |
| RSAD2_ISG15_HERC6_FAM20A | 0.862 | 0.852 | 0.872 | 0.882 | 0.815 | 0.949 | 0.922 | 0.834 | 1 |
| SIGLEC1_SLC1A2_RETN_MMP8 | 0.862 | 0.852 | 0.871 | 0.892 | 0.828 | 0.957 | 0.951 | 0.904 | 0.998 |
| ISG15_HERC6_SLC1A2_FAM20A | 0.862 | 0.852 | 0.871 | 0.886 | 0.822 | 0.950 | 0.933 | 0.850 | 1 |
| IFIT1_ISG15_HERC6_RETN | 0.862 | 0.851 | 0.872 | 0.883 | 0.811 | 0.955 | 0.938 | 0.881 | 0.995 |
| IFI44L_IL1R2_OLAH_RETN | 0.862 | 0.852 | 0.871 | 0.890 | 0.826 | 0.955 | 0.945 | 0.893 | 0.996 |
| IFIT1_SIGLEC1_SLC1A2_MMP8 | 0.862 | 0.853 | 0.870 | 0.885 | 0.821 | 0.949 | 0.932 | 0.874 | 0.989 |
| IFIT1_SIGLEC1_ISG15_RETN | 0.862 | 0.852 | 0.871 | 0.881 | 0.807 | 0.954 | 0.947 | 0.895 | 0.998 |
| RSAD2_OAS1_IL1R2_RETN | 0.861 | 0.852 | 0.871 | 0.885 | 0.816 | 0.954 | 0.941 | 0.889 | 0.994 |
| IFI27_IFI44L_ISG15_MMP8 | 0.861 | 0.852 | 0.871 | 0.880 | 0.810 | 0.950 | 0.905 | 0.829 | 0.982 |
| RSAD2_IFIT1_SIGLEC1_RETN | 0.861 | 0.851 | 0.871 | 0.879 | 0.804 | 0.954 | 0.941 | 0.887 | 0.996 |
| OAS1_IFIT1_OLAH_RETN | 0.861 | 0.851 | 0.871 | 0.883 | 0.808 | 0.958 | 0.948 | 0.898 | 0.998 |
| RSAD2_OAS1_IL1R2_MMP8 | 0.861 | 0.852 | 0.870 | 0.887 | 0.822 | 0.951 | 0.936 | 0.881 | 0.991 |
| IFI27_OAS1_IFIT1_SIGLEC1 | 0.861 | 0.852 | 0.871 | 0.883 | 0.819 | 0.946 | 0.940 | 0.885 | 0.995 |
| RSAD2_OAS1_ISG15_IL1R2 | 0.861 | 0.852 | 0.870 | 0.886 | 0.821 | 0.952 | 0.940 | 0.887 | 0.993 |
| OAS1_IFI44L_IL1R2_RETN | 0.861 | 0.852 | 0.871 | 0.884 | 0.815 | 0.953 | 0.940 | 0.887 | 0.994 |
| OAS1_ISG15_IL1R2_RETN | 0.861 | 0.852 | 0.870 | 0.888 | 0.821 | 0.956 | 0.941 | 0.888 | 0.994 |
| OAS1_SIGLEC1_SLC1A2_MMP8 | 0.861 | 0.852 | 0.870 | 0.884 | 0.820 | 0.949 | 0.937 | 0.882 | 0.992 |
| RSAD2_IFI27_IFI44L_SIGLEC1 | 0.861 | 0.851 | 0.871 | 0.886 | 0.824 | 0.947 | 0.941 | 0.887 | 0.996 |
| IFI27_IFIT1_SIGLEC1_ISG15 | 0.861 | 0.851 | 0.871 | 0.884 | 0.822 | 0.946 | 0.939 | 0.882 | 0.996 |
| IFI27_IFIT1_ISG15_MMP8 | 0.861 | 0.851 | 0.870 | 0.877 | 0.807 | 0.948 | 0.912 | 0.838 | 0.986 |
| RSAD2_IFI44L_ISG15_IL1R2 | 0.861 | 0.852 | 0.870 | 0.885 | 0.821 | 0.949 | 0.958 | 0.915 | 1.000 |
| ISG15_HERC6_FAM20A_MMP8 | 0.861 | 0.850 | 0.871 | 0.882 | 0.812 | 0.953 | 0.929 | 0.848 | 1 |
| RSAD2_OAS1_OLAH_RETN | 0.861 | 0.851 | 0.871 | 0.884 | 0.809 | 0.958 | 0.947 | 0.896 | 0.998 |
| RSAD2_IFI27_SIGLEC1_ISG15 | 0.861 | 0.851 | 0.870 | 0.884 | 0.822 | 0.947 | 0.940 | 0.885 | 0.996 |
| OAS1_ISG15_IL1R2_MMP8 | 0.860 | 0.852 | 0.869 | 0.886 | 0.822 | 0.950 | 0.930 | 0.871 | 0.990 |
| OAS1_IL1R2_RETN_MMP8 | 0.860 | 0.851 | 0.870 | 0.886 | 0.818 | 0.955 | 0.946 | 0.897 | 0.995 |
| IFI27_OAS1_IFI44L_SIGLEC1 | 0.860 | 0.851 | 0.870 | 0.885 | 0.821 | 0.948 | 0.948 | 0.897 | 0.999 |
| IFI44L_HERC6_SLC1A2_FAM20A | 0.860 | 0.850 | 0.870 | 0.885 | 0.820 | 0.950 | 0.935 | 0.854 | 1 |
| RSAD2_OAS1_OLAH_MMP8 | 0.860 | 0.851 | 0.870 | 0.879 | 0.807 | 0.952 | 0.945 | 0.890 | 0.999 |
| RSAD2_SIGLEC1_SLC1A2_MMP8 | 0.860 | 0.851 | 0.869 | 0.885 | 0.821 | 0.949 | 0.936 | 0.881 | 0.991 |
| RSAD2_IFI27_OAS1_SIGLEC1 | 0.860 | 0.850 | 0.870 | 0.885 | 0.822 | 0.947 | 0.943 | 0.889 | 0.998 |
| ISG15_HERC6_SLC1A2_RETN | 0.860 | 0.851 | 0.870 | 0.886 | 0.819 | 0.954 | 0.940 | 0.877 | 1 |
| RSAD2_ISG15_IL1R2_RETN | 0.860 | 0.851 | 0.869 | 0.887 | 0.818 | 0.955 | 0.955 | 0.912 | 0.999 |
| IFIT1_ISG15_HERC6_FAM20A | 0.860 | 0.850 | 0.870 | 0.880 | 0.811 | 0.949 | 0.926 | 0.839 | 1 |
| IFI44L_IL1R2_RETN_MMP8 | 0.860 | 0.850 | 0.870 | 0.886 | 0.819 | 0.953 | 0.963 | 0.925 | 1 |
| ISG15_IL1R2_RETN_MMP8 | 0.860 | 0.851 | 0.869 | 0.888 | 0.825 | 0.950 | 0.935 | 0.871 | 0.999 |
| RSAD2_IFI44L_IL1R2_MMP8 | 0.860 | 0.851 | 0.869 | 0.884 | 0.818 | 0.949 | 0.950 | 0.904 | 0.996 |
| IFI44L_ISG15_HERC6_RETN | 0.860 | 0.850 | 0.870 | 0.879 | 0.803 | 0.955 | 0.951 | 0.901 | 1 |
| SIGLEC1_ISG15_SLC1A2_MMP8 | 0.860 | 0.851 | 0.869 | 0.884 | 0.818 | 0.949 | 0.936 | 0.881 | 0.991 |
| IFIT1_IFI44L_SIGLEC1_RETN | 0.860 | 0.850 | 0.870 | 0.877 | 0.803 | 0.952 | 0.936 | 0.878 | 0.994 |
| IFI27_OAS1_SIGLEC1_ISG15 | 0.860 | 0.850 | 0.869 | 0.882 | 0.818 | 0.946 | 0.942 | 0.889 | 0.996 |
| RSAD2_IFI44L_IL1R2_RETN | 0.860 | 0.850 | 0.869 | 0.883 | 0.814 | 0.952 | 0.953 | 0.909 | 0.998 |
| IFI27_IFIT1_IFI44L_SIGLEC1 | 0.860 | 0.850 | 0.869 | 0.885 | 0.821 | 0.950 | 0.946 | 0.894 | 0.997 |
| IFI44L_SIGLEC1_ISG15_RETN | 0.860 | 0.849 | 0.870 | 0.877 | 0.801 | 0.953 | 0.952 | 0.906 | 0.998 |
| RSAD2_ISG15_IL1R2_MMP8 | 0.860 | 0.851 | 0.868 | 0.883 | 0.818 | 0.948 | 0.951 | 0.904 | 0.999 |
| SLC1A2_IL1R2_OLAH_FAM20A | 0.859 | 0.850 | 0.869 | 0.887 | 0.827 | 0.947 | 0.858 | 0.752 | 0.963 |
| ISG15_OLAH_FAM20A_MMP8 | 0.859 | 0.850 | 0.869 | 0.886 | 0.820 | 0.952 | 0.947 | 0.894 | 0.999 |
| IFIT1_OLAH_FAM20A_RETN | 0.859 | 0.849 | 0.870 | 0.881 | 0.808 | 0.955 | 0.958 | 0.909 | 1 |
| RSAD2_IFI27_IFIT1_SIGLEC1 | 0.859 | 0.849 | 0.869 | 0.884 | 0.821 | 0.946 | 0.942 | 0.888 | 0.997 |
| OAS1_IFIT1_OLAH_MMP8 | 0.859 | 0.849 | 0.869 | 0.879 | 0.806 | 0.952 | 0.945 | 0.893 | 0.997 |
| RSAD2_OAS1_SIGLEC1_RETN | 0.859 | 0.849 | 0.869 | 0.874 | 0.797 | 0.951 | 0.945 | 0.893 | 0.996 |
| IFI44L_ISG15_SLC1A2_FAM20A | 0.859 | 0.849 | 0.869 | 0.882 | 0.813 | 0.952 | 0.932 | 0.853 | 1 |
| OAS1_OLAH_RETN_MMP8 | 0.859 | 0.849 | 0.869 | 0.883 | 0.809 | 0.958 | 0.959 | 0.917 | 1 |
| SLC1A2_OLAH_FAM20A_MMP8 | 0.859 | 0.849 | 0.869 | 0.890 | 0.832 | 0.949 | 0.873 | 0.777 | 0.969 |
| RSAD2_OAS1_IFIT1_OLAH | 0.859 | 0.849 | 0.869 | 0.880 | 0.806 | 0.953 | 0.941 | 0.887 | 0.996 |
| IFIT1_HERC6_FAM20A_MMP8 | 0.859 | 0.848 | 0.869 | 0.879 | 0.809 | 0.949 | 0.925 | 0.842 | 1 |
| ISG15_OLAH_FAM20A_RETN | 0.859 | 0.849 | 0.868 | 0.887 | 0.819 | 0.955 | 0.945 | 0.891 | 0.998 |
| IFI27_OAS1_ISG15_HERC6 | 0.859 | 0.849 | 0.869 | 0.882 | 0.823 | 0.942 | 0.904 | 0.834 | 0.975 |
| IFI27_OAS1_IFIT1_MMP8 | 0.859 | 0.849 | 0.868 | 0.874 | 0.803 | 0.946 | 0.913 | 0.839 | 0.987 |
| RSAD2_IL1R2_RETN_MMP8 | 0.858 | 0.849 | 0.868 | 0.882 | 0.813 | 0.952 | 0.961 | 0.921 | 1 |
| OAS1_SIGLEC1_ISG15_RETN | 0.858 | 0.848 | 0.869 | 0.877 | 0.801 | 0.952 | 0.952 | 0.904 | 1 |
| IFIT1_IFI44L_HERC6_FAM20A | 0.858 | 0.848 | 0.869 | 0.877 | 0.805 | 0.949 | 0.928 | 0.845 | 1 |
| OAS1_ISG15_HERC6_FAM20A | 0.858 | 0.848 | 0.868 | 0.876 | 0.806 | 0.947 | 0.926 | 0.839 | 1 |
| OAS1_IFI44L_OLAH_RETN | 0.858 | 0.848 | 0.869 | 0.881 | 0.806 | 0.956 | 0.948 | 0.900 | 0.996 |
| OAS1_ISG15_OLAH_RETN | 0.858 | 0.848 | 0.868 | 0.884 | 0.810 | 0.957 | 0.953 | 0.907 | 0.999 |
| RSAD2_IFI27_IFI44L_MMP8 | 0.858 | 0.848 | 0.868 | 0.878 | 0.808 | 0.948 | 0.909 | 0.833 | 0.985 |
| IFI27_OAS1_ISG15_MMP8 | 0.858 | 0.848 | 0.868 | 0.876 | 0.806 | 0.947 | 0.903 | 0.825 | 0.981 |
| IFI27_IFIT1_IFI44L_MMP8 | 0.858 | 0.848 | 0.868 | 0.877 | 0.807 | 0.948 | 0.912 | 0.838 | 0.986 |
| IFIT1_SIGLEC1_RETN_MMP8 | 0.858 | 0.848 | 0.868 | 0.878 | 0.804 | 0.953 | 0.947 | 0.895 | 0.998 |
| RSAD2_IFI44L_HERC6_RETN | 0.858 | 0.848 | 0.868 | 0.877 | 0.800 | 0.954 | 0.926 | 0.866 | 0.986 |
| OAS1_IFI44L_SIGLEC1_RETN | 0.858 | 0.848 | 0.868 | 0.877 | 0.800 | 0.954 | 0.947 | 0.896 | 0.998 |
| IFI44L_OLAH_RETN_MMP8 | 0.858 | 0.848 | 0.868 | 0.880 | 0.806 | 0.953 | 0.952 | 0.905 | 0.999 |
| IFI44L_ISG15_HERC6_FAM20A | 0.858 | 0.848 | 0.868 | 0.873 | 0.800 | 0.946 | 0.934 | 0.850 | 1 |
| IFI27_OAS1_IFI44L_MMP8 | 0.858 | 0.848 | 0.868 | 0.877 | 0.806 | 0.947 | 0.908 | 0.831 | 0.984 |
| RSAD2_SIGLEC1_ISG15_RETN | 0.858 | 0.847 | 0.868 | 0.878 | 0.803 | 0.953 | 0.949 | 0.899 | 0.998 |
| IFI44L_SIGLEC1_HERC6_SLC1A2 | 0.857 | 0.849 | 0.866 | 0.880 | 0.814 | 0.945 | 0.951 | 0.903 | 0.999 |
| IFI44L_ISG15_OLAH_RETN | 0.857 | 0.848 | 0.867 | 0.881 | 0.807 | 0.954 | 0.948 | 0.898 | 0.997 |
| IFIT1_HERC6_SLC1A2_FAM20A | 0.857 | 0.847 | 0.867 | 0.882 | 0.817 | 0.947 | 0.929 | 0.845 | 1 |
| ISG15_HERC6_RETN_MMP8 | 0.857 | 0.847 | 0.868 | 0.877 | 0.801 | 0.954 | 0.945 | 0.893 | 0.996 |
| OAS1_IFIT1_ISG15_OLAH | 0.857 | 0.848 | 0.867 | 0.879 | 0.806 | 0.952 | 0.945 | 0.893 | 0.997 |
| IFI44L_SIGLEC1_RETN_MMP8 | 0.857 | 0.847 | 0.868 | 0.874 | 0.797 | 0.951 | 0.952 | 0.905 | 1.000 |
| OAS1_HERC6_SLC1A2_FAM20A | 0.857 | 0.847 | 0.867 | 0.884 | 0.819 | 0.949 | 0.930 | 0.846 | 1 |
| RSAD2_OAS1_IFI44L_OLAH | 0.857 | 0.848 | 0.867 | 0.883 | 0.811 | 0.954 | 0.939 | 0.882 | 0.996 |
| OAS1_IFIT1_IFI44L_OLAH | 0.857 | 0.848 | 0.867 | 0.882 | 0.810 | 0.954 | 0.941 | 0.887 | 0.996 |
| RSAD2_HERC6_SLC1A2_FAM20A | 0.857 | 0.847 | 0.867 | 0.886 | 0.823 | 0.949 | 0.930 | 0.846 | 1 |
| IFI44L_SLC1A2_FAM20A_MMP8 | 0.857 | 0.847 | 0.867 | 0.882 | 0.814 | 0.951 | 0.925 | 0.844 | 1 |
| SIGLEC1_ISG15_RETN_MMP8 | 0.857 | 0.847 | 0.867 | 0.874 | 0.797 | 0.952 | 0.958 | 0.914 | 1 |
| OAS1_IFI44L_HERC6_RETN | 0.857 | 0.847 | 0.867 | 0.876 | 0.798 | 0.953 | 0.939 | 0.885 | 0.994 |
| IFI44L_HERC6_SLC1A2_RETN | 0.857 | 0.847 | 0.867 | 0.884 | 0.814 | 0.953 | 0.938 | 0.874 | 1 |
| OAS1_IFI44L_OLAH_MMP8 | 0.857 | 0.847 | 0.867 | 0.880 | 0.807 | 0.952 | 0.941 | 0.887 | 0.995 |
| RSAD2_OAS1_ISG15_OLAH | 0.857 | 0.847 | 0.866 | 0.881 | 0.808 | 0.953 | 0.945 | 0.891 | 0.998 |
| RSAD2_IFI27_OAS1_MMP8 | 0.857 | 0.847 | 0.867 | 0.877 | 0.806 | 0.947 | 0.908 | 0.830 | 0.985 |
| IFI27_SIGLEC1_ISG15_HERC6 | 0.857 | 0.846 | 0.867 | 0.882 | 0.818 | 0.946 | 0.948 | 0.898 | 0.997 |
| IFI44L_ISG15_FAM20A_RETN | 0.857 | 0.846 | 0.867 | 0.874 | 0.800 | 0.949 | 0.942 | 0.874 | 1 |
| IFI44L_ISG15_OLAH_MMP8 | 0.856 | 0.847 | 0.866 | 0.876 | 0.805 | 0.946 | 0.943 | 0.893 | 0.994 |
| RSAD2_IFI44L_SIGLEC1_RETN | 0.856 | 0.846 | 0.867 | 0.875 | 0.798 | 0.952 | 0.945 | 0.893 | 0.996 |
| IFI44L_SIGLEC1_HERC6_MMP8 | 0.856 | 0.846 | 0.866 | 0.874 | 0.800 | 0.947 | 0.914 | 0.846 | 0.982 |
| RSAD2_IFI27_ISG15_MMP8 | 0.856 | 0.846 | 0.866 | 0.877 | 0.807 | 0.948 | 0.908 | 0.831 | 0.985 |
| SLC1A2_OLAH_FAM20A_RETN | 0.856 | 0.846 | 0.866 | 0.883 | 0.821 | 0.945 | 0.862 | 0.759 | 0.965 |
| OAS1_IFIT1_SIGLEC1_SLC1A2 | 0.856 | 0.847 | 0.865 | 0.877 | 0.812 | 0.942 | 0.949 | 0.902 | 0.996 |
| OAS1_ISG15_OLAH_MMP8 | 0.856 | 0.846 | 0.866 | 0.880 | 0.807 | 0.952 | 0.948 | 0.897 | 0.999 |
| RSAD2_OAS1_HERC6_FAM20A | 0.856 | 0.846 | 0.866 | 0.875 | 0.802 | 0.947 | 0.921 | 0.833 | 1 |
| RSAD2_HERC6_FAM20A_MMP8 | 0.856 | 0.845 | 0.866 | 0.879 | 0.809 | 0.950 | 0.924 | 0.841 | 1 |
| OAS1_SIGLEC1_RETN_MMP8 | 0.856 | 0.845 | 0.866 | 0.874 | 0.798 | 0.951 | 0.951 | 0.902 | 1 |
| RSAD2_IFI44L_HERC6_FAM20A | 0.856 | 0.845 | 0.866 | 0.880 | 0.810 | 0.950 | 0.926 | 0.847 | 1 |
| IFI27_OAS1_HERC6_SLC1A2 | 0.856 | 0.846 | 0.865 | 0.882 | 0.820 | 0.943 | 0.915 | 0.840 | 0.991 |
| IFI44L_HERC6_FAM20A_MMP8 | 0.856 | 0.845 | 0.866 | 0.876 | 0.804 | 0.948 | 0.932 | 0.851 | 1 |
| IFIT1_IFI44L_HERC6_RETN | 0.856 | 0.845 | 0.866 | 0.874 | 0.795 | 0.952 | 0.927 | 0.865 | 0.989 |
| OAS1_HERC6_SLC1A2_RETN | 0.856 | 0.846 | 0.865 | 0.884 | 0.817 | 0.952 | 0.925 | 0.855 | 0.995 |
| IFIT1_ISG15_OLAH_RETN | 0.856 | 0.846 | 0.866 | 0.884 | 0.811 | 0.956 | 0.962 | 0.921 | 1 |
| RSAD2_SIGLEC1_HERC6_MMP8 | 0.855 | 0.846 | 0.865 | 0.875 | 0.805 | 0.945 | 0.901 | 0.829 | 0.973 |
| RSAD2_IFI27_ISG15_HERC6 | 0.855 | 0.846 | 0.865 | 0.880 | 0.820 | 0.940 | 0.882 | 0.801 | 0.962 |
| IFIT1_HERC6_SLC1A2_RETN | 0.855 | 0.845 | 0.865 | 0.884 | 0.816 | 0.953 | 0.937 | 0.872 | 1 |
| OAS1_ISG15_FAM20A_RETN | 0.855 | 0.845 | 0.866 | 0.882 | 0.809 | 0.955 | 0.940 | 0.863 | 1 |
| RSAD2_IFI27_HERC6_SLC1A2 | 0.855 | 0.846 | 0.865 | 0.880 | 0.818 | 0.942 | 0.912 | 0.835 | 0.988 |
| RSAD2_SIGLEC1_RETN_MMP8 | 0.855 | 0.844 | 0.866 | 0.876 | 0.800 | 0.952 | 0.951 | 0.903 | 0.999 |
| RSAD2_IFI27_IFIT1_MMP8 | 0.855 | 0.845 | 0.865 | 0.875 | 0.804 | 0.946 | 0.911 | 0.836 | 0.986 |
| IFI27_IFI44L_HERC6_SLC1A2 | 0.855 | 0.845 | 0.864 | 0.882 | 0.820 | 0.945 | 0.923 | 0.853 | 0.993 |
| RSAD2_IFI27_IFI44L_HERC6 | 0.855 | 0.845 | 0.865 | 0.880 | 0.820 | 0.939 | 0.891 | 0.816 | 0.967 |
| OAS1_IFIT1_SIGLEC1_MMP8 | 0.855 | 0.845 | 0.864 | 0.871 | 0.798 | 0.945 | 0.905 | 0.835 | 0.976 |
| OAS1_IFIT1_HERC6_RETN | 0.854 | 0.844 | 0.865 | 0.872 | 0.795 | 0.949 | 0.934 | 0.873 | 0.994 |
| IFIT1_SIGLEC1_ISG15_SLC1A2 | 0.854 | 0.846 | 0.863 | 0.878 | 0.813 | 0.943 | 0.950 | 0.904 | 0.996 |
| RSAD2_OLAH_RETN_MMP8 | 0.854 | 0.844 | 0.865 | 0.877 | 0.801 | 0.952 | 0.965 | 0.927 | 1 |
| RSAD2_OAS1_FAM20A_RETN | 0.854 | 0.844 | 0.865 | 0.880 | 0.807 | 0.954 | 0.929 | 0.847 | 1 |
| OAS1_HERC6_FAM20A_MMP8 | 0.854 | 0.844 | 0.865 | 0.878 | 0.807 | 0.950 | 0.927 | 0.845 | 1 |
| RSAD2_ISG15_OLAH_RETN | 0.854 | 0.844 | 0.864 | 0.877 | 0.801 | 0.952 | 0.966 | 0.929 | 1 |
| OAS1_SIGLEC1_HERC6_MMP8 | 0.854 | 0.845 | 0.864 | 0.875 | 0.806 | 0.944 | 0.914 | 0.847 | 0.981 |
| RSAD2_HERC6_SLC1A2_RETN | 0.854 | 0.845 | 0.864 | 0.885 | 0.817 | 0.952 | 0.930 | 0.863 | 0.997 |
| SIGLEC1_ISG15_HERC6_SLC1A2 | 0.854 | 0.846 | 0.863 | 0.880 | 0.814 | 0.946 | 0.950 | 0.901 | 0.999 |
| IFIT1_OLAH_RETN_MMP8 | 0.854 | 0.844 | 0.865 | 0.876 | 0.799 | 0.952 | 0.966 | 0.929 | 1 |
| IFIT1_IFI44L_OLAH_MMP8 | 0.854 | 0.844 | 0.864 | 0.874 | 0.801 | 0.947 | 0.946 | 0.895 | 0.996 |
| RSAD2_IFIT1_OLAH_RETN | 0.854 | 0.844 | 0.864 | 0.881 | 0.805 | 0.957 | 0.958 | 0.914 | 1 |
| RSAD2_OAS1_HERC6_RETN | 0.854 | 0.844 | 0.864 | 0.871 | 0.793 | 0.948 | 0.927 | 0.864 | 0.990 |
| OAS1_IFI44L_ISG15_OLAH | 0.854 | 0.844 | 0.864 | 0.882 | 0.811 | 0.953 | 0.945 | 0.893 | 0.996 |
| IFI27_ISG15_HERC6_SLC1A2 | 0.854 | 0.844 | 0.864 | 0.882 | 0.819 | 0.945 | 0.921 | 0.847 | 0.994 |
| OAS1_SLC1A2_FAM20A_RETN | 0.854 | 0.844 | 0.864 | 0.881 | 0.811 | 0.951 | 0.930 | 0.849 | 1 |
| IFIT1_IFI44L_SIGLEC1_SLC1A2 | 0.854 | 0.845 | 0.863 | 0.879 | 0.814 | 0.944 | 0.947 | 0.899 | 0.995 |
| IFI27_IFIT1_HERC6_SLC1A2 | 0.854 | 0.844 | 0.864 | 0.883 | 0.819 | 0.946 | 0.939 | 0.876 | 1 |
| RSAD2_IFI27_IFIT1_HERC6 | 0.854 | 0.843 | 0.864 | 0.882 | 0.822 | 0.943 | 0.912 | 0.844 | 0.980 |
| RSAD2_IFIT1_SIGLEC1_SLC1A2 | 0.854 | 0.845 | 0.863 | 0.878 | 0.812 | 0.943 | 0.950 | 0.904 | 0.996 |
| IFIT1_IFI44L_OLAH_RETN | 0.854 | 0.843 | 0.864 | 0.879 | 0.803 | 0.954 | 0.947 | 0.897 | 0.997 |
| IFIT1_IFI44L_ISG15_OLAH | 0.853 | 0.844 | 0.863 | 0.877 | 0.806 | 0.949 | 0.946 | 0.896 | 0.995 |
| OAS1_SIGLEC1_ISG15_SLC1A2 | 0.853 | 0.845 | 0.862 | 0.877 | 0.812 | 0.942 | 0.952 | 0.907 | 0.997 |
| RSAD2_OAS1_SIGLEC1_SLC1A2 | 0.853 | 0.844 | 0.863 | 0.876 | 0.810 | 0.941 | 0.948 | 0.900 | 0.996 |
| IFI27_OAS1_IFI44L_HERC6 | 0.853 | 0.843 | 0.864 | 0.879 | 0.818 | 0.940 | 0.904 | 0.834 | 0.975 |
| IFIT1_ISG15_OLAH_MMP8 | 0.853 | 0.844 | 0.863 | 0.874 | 0.801 | 0.947 | 0.961 | 0.921 | 1 |
| ISG15_OLAH_RETN_MMP8 | 0.853 | 0.843 | 0.863 | 0.879 | 0.811 | 0.948 | 0.957 | 0.912 | 1 |
| IFI27_OAS1_IFIT1_HERC6 | 0.853 | 0.843 | 0.863 | 0.880 | 0.818 | 0.942 | 0.925 | 0.862 | 0.988 |
| OAS1_IFI44L_SIGLEC1_SLC1A2 | 0.853 | 0.844 | 0.862 | 0.876 | 0.811 | 0.942 | 0.952 | 0.907 | 0.997 |
| HERC6_SLC1A2_RETN_MMP8 | 0.853 | 0.843 | 0.863 | 0.882 | 0.814 | 0.950 | 0.923 | 0.853 | 0.993 |
| RSAD2_IFI27_OAS1_HERC6 | 0.853 | 0.843 | 0.863 | 0.879 | 0.819 | 0.940 | 0.884 | 0.804 | 0.963 |
| OAS1_IFIT1_FAM20A_RETN | 0.853 | 0.843 | 0.863 | 0.877 | 0.801 | 0.952 | 0.935 | 0.853 | 1 |
| RSAD2_IFI44L_SIGLEC1_SLC1A2 | 0.853 | 0.844 | 0.862 | 0.878 | 0.813 | 0.943 | 0.946 | 0.897 | 0.995 |
| RSAD2_IFIT1_HERC6_RETN | 0.853 | 0.843 | 0.863 | 0.872 | 0.794 | 0.949 | 0.926 | 0.863 | 0.989 |
| IFI27_OAS1_IFIT1_SLC1A2 | 0.853 | 0.843 | 0.863 | 0.874 | 0.805 | 0.944 | 0.949 | 0.891 | 1 |
| RSAD2_SIGLEC1_ISG15_SLC1A2 | 0.853 | 0.844 | 0.861 | 0.878 | 0.813 | 0.943 | 0.948 | 0.900 | 0.995 |
| IFIT1_SLC1A2_FAM20A_RETN | 0.853 | 0.843 | 0.863 | 0.879 | 0.807 | 0.951 | 0.932 | 0.852 | 1 |
| IFI44L_SIGLEC1_ISG15_SLC1A2 | 0.853 | 0.844 | 0.861 | 0.879 | 0.813 | 0.945 | 0.952 | 0.907 | 0.997 |
| OAS1_IFI44L_HERC6_FAM20A | 0.853 | 0.842 | 0.863 | 0.872 | 0.799 | 0.945 | 0.935 | 0.852 | 1 |
| RSAD2_ISG15_OLAH_MMP8 | 0.853 | 0.843 | 0.862 | 0.877 | 0.805 | 0.948 | 0.953 | 0.908 | 0.998 |
| RSAD2_IFIT1_OLAH_MMP8 | 0.852 | 0.843 | 0.862 | 0.875 | 0.802 | 0.948 | 0.951 | 0.905 | 0.997 |
| IFI44L_HERC6_RETN_MMP8 | 0.852 | 0.842 | 0.863 | 0.873 | 0.793 | 0.952 | 0.921 | 0.856 | 0.985 |
| RSAD2_IFI44L_OLAH_RETN | 0.852 | 0.842 | 0.863 | 0.879 | 0.804 | 0.954 | 0.953 | 0.908 | 0.999 |
| ISG15_HERC6_SLC1A2_MMP8 | 0.852 | 0.843 | 0.861 | 0.881 | 0.814 | 0.947 | 0.905 | 0.828 | 0.983 |
| RSAD2_IFI44L_OLAH_MMP8 | 0.852 | 0.842 | 0.862 | 0.874 | 0.801 | 0.947 | 0.946 | 0.895 | 0.996 |
| IFIT1_IFI44L_SLC1A2_FAM20A | 0.852 | 0.842 | 0.862 | 0.878 | 0.809 | 0.947 | 0.923 | 0.840 | 1 |
| SLC1A2_IL1R2_OLAH_RETN | 0.852 | 0.843 | 0.861 | 0.879 | 0.818 | 0.941 | 0.840 | 0.729 | 0.951 |
| IFI27_IFI44L_ISG15_SLC1A2 | 0.852 | 0.842 | 0.861 | 0.879 | 0.812 | 0.946 | 0.929 | 0.866 | 0.992 |
| SIGLEC1_ISG15_HERC6_MMP8 | 0.852 | 0.842 | 0.862 | 0.871 | 0.795 | 0.946 | 0.915 | 0.849 | 0.981 |
| RSAD2_IFIT1_SIGLEC1_MMP8 | 0.852 | 0.841 | 0.862 | 0.871 | 0.798 | 0.945 | 0.907 | 0.836 | 0.977 |
| RSAD2_IFIT1_ISG15_OLAH | 0.852 | 0.842 | 0.861 | 0.877 | 0.804 | 0.949 | 0.957 | 0.914 | 0.999 |
| IFIT1_HERC6_RETN_MMP8 | 0.852 | 0.841 | 0.862 | 0.872 | 0.795 | 0.949 | 0.914 | 0.846 | 0.982 |
| OAS1_IFIT1_HERC6_FAM20A | 0.852 | 0.841 | 0.862 | 0.872 | 0.799 | 0.945 | 0.932 | 0.847 | 1 |
| SLC1A2_IL1R2_OLAH_MMP8 | 0.852 | 0.843 | 0.861 | 0.879 | 0.818 | 0.940 | 0.850 | 0.745 | 0.955 |
| OAS1_IFIT1_SIGLEC1_HERC6 | 0.852 | 0.842 | 0.861 | 0.869 | 0.802 | 0.936 | 0.911 | 0.844 | 0.978 |
| RSAD2_IFI44L_SLC1A2_FAM20A | 0.851 | 0.841 | 0.862 | 0.880 | 0.812 | 0.949 | 0.920 | 0.838 | 1 |
| ISG15_SLC1A2_FAM20A_RETN | 0.851 | 0.841 | 0.861 | 0.880 | 0.812 | 0.948 | 0.921 | 0.837 | 1 |
| IFI44L_HERC6_SLC1A2_MMP8 | 0.851 | 0.842 | 0.860 | 0.877 | 0.809 | 0.944 | 0.904 | 0.828 | 0.981 |
| OAS1_IFI44L_FAM20A_RETN | 0.851 | 0.841 | 0.862 | 0.875 | 0.800 | 0.951 | 0.939 | 0.863 | 1 |
| RSAD2_IFI27_IFI44L_SLC1A2 | 0.851 | 0.841 | 0.861 | 0.877 | 0.809 | 0.944 | 0.925 | 0.862 | 0.988 |
| IFIT1_ISG15_SLC1A2_FAM20A | 0.851 | 0.840 | 0.861 | 0.873 | 0.800 | 0.946 | 0.937 | 0.857 | 1 |
| IFI27_IFI44L_ISG15_HERC6 | 0.851 | 0.840 | 0.861 | 0.877 | 0.815 | 0.940 | 0.909 | 0.840 | 0.977 |
| IFI27_IFIT1_ISG15_SLC1A2 | 0.851 | 0.841 | 0.861 | 0.877 | 0.808 | 0.945 | 0.949 | 0.893 | 1 |
| RSAD2_IFI44L_ISG15_OLAH | 0.850 | 0.841 | 0.860 | 0.877 | 0.805 | 0.948 | 0.943 | 0.893 | 0.994 |
| OAS1_IFI44L_SLC1A2_FAM20A | 0.850 | 0.840 | 0.861 | 0.880 | 0.811 | 0.949 | 0.929 | 0.847 | 1 |
| RSAD2_IFIT1_IFI44L_OLAH | 0.850 | 0.840 | 0.860 | 0.877 | 0.804 | 0.949 | 0.947 | 0.897 | 0.996 |
| IFIT1_HERC6_SLC1A2_MMP8 | 0.850 | 0.841 | 0.859 | 0.876 | 0.808 | 0.944 | 0.899 | 0.818 | 0.980 |
| SLC1A2_OLAH_RETN_MMP8 | 0.850 | 0.840 | 0.860 | 0.881 | 0.819 | 0.942 | 0.858 | 0.757 | 0.958 |
| OAS1_HERC6_RETN_MMP8 | 0.850 | 0.840 | 0.861 | 0.873 | 0.797 | 0.949 | 0.917 | 0.851 | 0.984 |
| RSAD2_IFI27_IFIT1_SLC1A2 | 0.850 | 0.840 | 0.860 | 0.875 | 0.806 | 0.944 | 0.948 | 0.890 | 1 |

Performance obtained for the identification of the viral or bacterial nature of the infection on the basis of the five-gene combinations:

TABLE 9

| | AUC | | | AUC | CI 95% | | AUC | CI 95% | |
|---|---|---|---|---|---|---|---|---|---|
| Model | model | CI 95% | | Train | Train | | Test | Test | |
| IFI27_SIGLEC1_IL1R2_FAM20A_MMP8 | 0.910 | 0.902 | 0.918 | 0.930 | 0.877 | 0.983 | 0.957 | 0.889 | 1 |
| IFI27_IL1R2_OLAH_FAM20A_MMP8 | 0.907 | 0.899 | 0.916 | 0.927 | 0.875 | 0.980 | 0.924 | 0.847 | 1 |
| IFI27_OAS1_HERC6_OLAH_FAM20A | 0.907 | 0.899 | 0.916 | 0.927 | 0.873 | 0.982 | 0.929 | 0.857 | 1 |
| RSAD2_IFI27_HERC6_OLAH_FAM20A | 0.907 | 0.899 | 0.916 | 0.927 | 0.872 | 0.981 | 0.930 | 0.860 | 1 |
| IFI27_IFIT1_SIGLEC1_IL1R2_FAM20A | 0.907 | 0.899 | 0.915 | 0.926 | 0.870 | 0.982 | 0.949 | 0.886 | 1 |
| IFI27_IFIT1_SIGLEC1_OLAH_FAM20A | 0.907 | 0.898 | 0.915 | 0.925 | 0.866 | 0.984 | 0.953 | 0.898 | 1 |
| IFI27_IFIT1_HERC6_OLAH_FAM20A | 0.907 | 0.898 | 0.915 | 0.925 | 0.869 | 0.981 | 0.934 | 0.866 | 1 |
| IFI27_ISG15_HERC6_OLAH_FAM20A | 0.906 | 0.898 | 0.915 | 0.925 | 0.868 | 0.982 | 0.936 | 0.870 | 1 |
| IFI27_HERC6_IL1R2_OLAH_FAM20A | 0.906 | 0.898 | 0.914 | 0.926 | 0.872 | 0.981 | 0.936 | 0.865 | 1 |
| IFI27_IFI44L_SIGLEC1_IL1R2_FAM20A | 0.906 | 0.898 | 0.914 | 0.927 | 0.873 | 0.982 | 0.950 | 0.884 | 1 |
| IFI27_OAS1_SIGLEC1_IL1R2_FAM20A | 0.906 | 0.898 | 0.914 | 0.928 | 0.872 | 0.983 | 0.951 | 0.888 | 1 |
| RSAD2_IFI27_SIGLEC1_IL1R2_FAM20A | 0.906 | 0.898 | 0.914 | 0.929 | 0.874 | 0.984 | 0.947 | 0.879 | 1 |
| IFI27_SIGLEC1_HERC6_IL1R2_FAM20A | 0.906 | 0.897 | 0.914 | 0.926 | 0.870 | 0.982 | 0.951 | 0.884 | 1 |
| IFI27_IFI44L_IL1R2_FAM20A_MMP8 | 0.905 | 0.897 | 0.913 | 0.927 | 0.874 | 0.979 | 0.927 | 0.844 | 1 |
| IFI27_SIGLEC1_ISG15_IL1R2_FAM20A | 0.905 | 0.897 | 0.913 | 0.927 | 0.873 | 0.982 | 0.950 | 0.883 | 1 |
| IFI27_ISG15_OLAH_FAM20A_MMP8 | 0.905 | 0.896 | 0.914 | 0.921 | 0.862 | 0.979 | 0.928 | 0.856 | 1 |
| IFI27_IFI44L_HERC6_OLAH_FAM20A | 0.905 | 0.896 | 0.914 | 0.923 | 0.865 | 0.981 | 0.941 | 0.877 | 1 |
| IFI27_SIGLEC1_ISG15_OLAH_FAM20A | 0.905 | 0.896 | 0.914 | 0.922 | 0.861 | 0.983 | 0.950 | 0.891 | 1 |
| IFI27_HERC6_OLAH_FAM20A_MMP8 | 0.905 | 0.896 | 0.914 | 0.923 | 0.864 | 0.981 | 0.941 | 0.877 | 1 |
| IFI27_OAS1_SIGLEC1_OLAH_FAM20A | 0.905 | 0.896 | 0.913 | 0.923 | 0.862 | 0.983 | 0.950 | 0.892 | 1 |
| IFI27_ISG15_IL1R2_OLAH_FAM20A | 0.905 | 0.897 | 0.913 | 0.924 | 0.868 | 0.981 | 0.925 | 0.850 | 1.000 |
| IFI27_SIGLEC1_OLAH_FAM20A_MMP8 | 0.905 | 0.896 | 0.913 | 0.921 | 0.859 | 0.983 | 0.953 | 0.895 | 1 |
| RSAD2_IFI27_HERC6_IL1R2_FAM20A | 0.905 | 0.897 | 0.913 | 0.928 | 0.878 | 0.978 | 0.927 | 0.845 | 1 |
| IFI27_OAS1_HERC6_IL1R2_FAM20A | 0.904 | 0.897 | 0.912 | 0.928 | 0.878 | 0.978 | 0.922 | 0.836 | 1 |
| IFI27_HERC6_IL1R2_FAM20A_MMP8 | 0.904 | 0.896 | 0.912 | 0.929 | 0.879 | 0.979 | 0.925 | 0.840 | 1 |
| IFI27_IFI44L_HERC6_IL1R2_FAM20A | 0.904 | 0.896 | 0.912 | 0.927 | 0.875 | 0.978 | 0.930 | 0.849 | 1 |
| IFI27_IFI44L_SIGLEC1_OLAH_FAM20A | 0.904 | 0.896 | 0.913 | 0.921 | 0.860 | 0.982 | 0.953 | 0.894 | 1 |
| IFI27_SIGLEC1_IL1R2_OLAH_FAM20A | 0.904 | 0.896 | 0.912 | 0.924 | 0.866 | 0.982 | 0.950 | 0.890 | 1 |
| RSAD2_IFI27_SIGLEC1_OLAH_FAM20A | 0.904 | 0.895 | 0.912 | 0.926 | 0.867 | 0.985 | 0.950 | 0.891 | 1 |
| IFI27_IFIT1_HERC6_IL1R2_FAM20A | 0.904 | 0.896 | 0.912 | 0.927 | 0.876 | 0.978 | 0.932 | 0.852 | 1 |
| IFI27_SIGLEC1_HERC6_OLAH_FAM20A | 0.903 | 0.895 | 0.912 | 0.919 | 0.856 | 0.981 | 0.949 | 0.888 | 1 |
| IFI27_HERC6_IL1R2_FAM20A_RETN | 0.903 | 0.895 | 0.912 | 0.931 | 0.880 | 0.982 | 0.929 | 0.849 | 1 |
| IFI27_ISG15_HERC6_IL1R2_FAM20A | 0.903 | 0.895 | 0.911 | 0.927 | 0.876 | 0.978 | 0.927 | 0.845 | 1 |
| IFI27_IFI44L_IL1R2_OLAH_FAM20A | 0.903 | 0.895 | 0.911 | 0.924 | 0.866 | 0.981 | 0.930 | 0.858 | 1 |
| IFI27_IFI44L_OLAH_FAM20A_MMP8 | 0.903 | 0.894 | 0.912 | 0.922 | 0.862 | 0.981 | 0.939 | 0.875 | 1 |
| IFI27_SIGLEC1_IL1R2_FAM20A_RETN | 0.903 | 0.895 | 0.911 | 0.926 | 0.871 | 0.982 | 0.949 | 0.881 | 1 |
| IFI27_IFIT1_IL1R2_FAM20A_MMP8 | 0.903 | 0.895 | 0.911 | 0.926 | 0.874 | 0.978 | 0.935 | 0.857 | 1 |
| IFI27_HERC6_OLAH_FAM20A_RETN | 0.902 | 0.894 | 0.911 | 0.922 | 0.863 | 0.982 | 0.942 | 0.878 | 1 |
| IFI27_IFIT1_OLAH_FAM20A_MMP8 | 0.902 | 0.893 | 0.911 | 0.920 | 0.860 | 0.981 | 0.941 | 0.879 | 1 |
| IFI27_OLAH_FAM20A_RETN_MMP8 | 0.902 | 0.893 | 0.911 | 0.923 | 0.865 | 0.981 | 0.929 | 0.857 | 1 |
| IFI27_IFI44L_ISG15_OLAH_FAM20A | 0.902 | 0.894 | 0.911 | 0.920 | 0.859 | 0.981 | 0.932 | 0.862 | 1 |
| IFI27_IFIT1_IL1R2_OLAH_FAM20A | 0.902 | 0.894 | 0.910 | 0.924 | 0.867 | 0.981 | 0.933 | 0.863 | 1 |
| IFI27_IL1R2_OLAH_FAM20A_RETN | 0.902 | 0.894 | 0.910 | 0.924 | 0.869 | 0.980 | 0.928 | 0.855 | 1 |
| IFI27_OAS1_SIGLEC1_HERC6_RETN | 0.902 | 0.893 | 0.910 | 0.919 | 0.859 | 0.978 | 0.948 | 0.887 | 1 |
| RSAD2_IFI27_OLAH_FAM20A_MMP8 | 0.902 | 0.893 | 0.910 | 0.922 | 0.863 | 0.980 | 0.935 | 0.867 | 1 |
| RSAD2_IFI27_IL1R2_OLAH_FAM20A | 0.902 | 0.893 | 0.910 | 0.924 | 0.868 | 0.980 | 0.930 | 0.858 | 1 |
| IFI27_IFIT1_SIGLEC1_HERC6_FAM20A | 0.902 | 0.894 | 0.910 | 0.920 | 0.861 | 0.980 | 0.941 | 0.866 | 1 |
| IFI27_OAS1_OLAH_FAM20A_MMP8 | 0.902 | 0.893 | 0.910 | 0.921 | 0.862 | 0.980 | 0.938 | 0.874 | 1 |
| IFI27_OAS1_IL1R2_OLAH_FAM20A | 0.901 | 0.893 | 0.909 | 0.923 | 0.866 | 0.980 | 0.933 | 0.862 | 1 |
| IFI27_IFIT1_IFI44L_OLAH_FAM20A | 0.901 | 0.893 | 0.910 | 0.920 | 0.859 | 0.981 | 0.939 | 0.873 | 1 |
| IFI27_ISG15_OLAH_FAM20A_RETN | 0.901 | 0.893 | 0.910 | 0.921 | 0.863 | 0.980 | 0.934 | 0.864 | 1 |
| IFI27_IFIT1_SIGLEC1_FAM20A_RETN | 0.901 | 0.893 | 0.910 | 0.919 | 0.859 | 0.979 | 0.948 | 0.880 | 1 |
| RSAD2_IFI27_IL1R2_FAM20A_MMP8 | 0.901 | 0.893 | 0.909 | 0.926 | 0.875 | 0.978 | 0.927 | 0.841 | 1 |
| IFI27_OAS1_IL1R2_FAM20A_MMP8 | 0.901 | 0.893 | 0.909 | 0.925 | 0.873 | 0.977 | 0.929 | 0.846 | 1 |
| IFI27_ISG15_IL1R2_FAM20A_MMP8 | 0.901 | 0.893 | 0.909 | 0.922 | 0.870 | 0.975 | 0.917 | 0.829 | 1 |
| IFI27_IFI44L_SIGLEC1_HERC6_OLAH | 0.901 | 0.893 | 0.909 | 0.925 | 0.872 | 0.978 | 0.946 | 0.895 | 0.997 |
| IFI27_IFI44L_ISG15_IL1R2_FAM20A | 0.901 | 0.893 | 0.909 | 0.924 | 0.870 | 0.978 | 0.927 | 0.845 | 1 |
| IFI27_IFI44L_IL1R2_FAM20A_RETN | 0.901 | 0.892 | 0.909 | 0.924 | 0.871 | 0.978 | 0.927 | 0.846 | 1 |
| IFI27_OAS1_ISG15_OLAH_FAM20A | 0.901 | 0.892 | 0.909 | 0.920 | 0.860 | 0.980 | 0.934 | 0.866 | 1 |
| IFI27_IFIT1_ISG15_OLAH_FAM20A | 0.901 | 0.892 | 0.909 | 0.920 | 0.860 | 0.980 | 0.935 | 0.867 | 1 |
| IFI27_SLC1A2_OLAH_FAM20A_MMP8 | 0.900 | 0.891 | 0.909 | 0.926 | 0.871 | 0.980 | 0.923 | 0.844 | 1 |
| IFI27_IFIT1_IFI44L_IL1R2_FAM20A | 0.900 | 0.892 | 0.908 | 0.922 | 0.867 | 0.976 | 0.930 | 0.851 | 1 |
| RSAD2_IFI27_HERC6_IL1R2_OLAH | 0.900 | 0.892 | 0.908 | 0.926 | 0.877 | 0.975 | 0.938 | 0.882 | 0.994 |
| IFI27_OAS1_IFI44L_OLAH_FAM20A | 0.900 | 0.891 | 0.909 | 0.920 | 0.860 | 0.981 | 0.936 | 0.867 | 1 |
| IFI27_SIGLEC1_OLAH_FAM20A_RETN | 0.900 | 0.891 | 0.909 | 0.918 | 0.856 | 0.981 | 0.951 | 0.891 | 1 |
| IFI27_OAS1_IFIT1_OLAH_FAM20A | 0.900 | 0.891 | 0.908 | 0.920 | 0.859 | 0.981 | 0.936 | 0.867 | 1 |
| IFI27_OAS1_HERC6_FAM20A_RETN | 0.900 | 0.891 | 0.909 | 0.923 | 0.865 | 0.981 | 0.934 | 0.853 | 1 |
| IFI27_ISG15_SLC1A2_OLAH_FAM20A | 0.900 | 0.891 | 0.909 | 0.927 | 0.873 | 0.981 | 0.917 | 0.836 | 0.998 |
| IFI27_OAS1_HERC6_OLAH_RETN | 0.900 | 0.891 | 0.908 | 0.923 | 0.867 | 0.978 | 0.939 | 0.878 | 1.000 |
| RSAD2_IFI27_SIGLEC1_HERC6_OLAH | 0.900 | 0.891 | 0.908 | 0.923 | 0.871 | 0.975 | 0.939 | 0.884 | 0.994 |
| IFI27_SLC1A2_IL1R2_OLAH_FAM20A | 0.900 | 0.891 | 0.908 | 0.927 | 0.875 | 0.980 | 0.918 | 0.835 | 1 |
| RSAD2_IFI27_HERC6_OLAH_MMP8 | 0.900 | 0.892 | 0.908 | 0.922 | 0.870 | 0.975 | 0.935 | 0.876 | 0.993 |
| IFI27_OAS1_HERC6_OLAH_MMP8 | 0.900 | 0.891 | 0.908 | 0.923 | 0.871 | 0.975 | 0.935 | 0.876 | 0.994 |
| RSAD2_IFI27_ISG15_OLAH_FAM20A | 0.900 | 0.891 | 0.908 | 0.918 | 0.857 | 0.979 | 0.934 | 0.866 | 1 |
| IFI27_OAS1_HERC6_IL1R2_OLAH | 0.900 | 0.892 | 0.907 | 0.927 | 0.879 | 0.976 | 0.938 | 0.880 | 0.996 |
| RSAD2_IFI27_OAS1_OLAH_FAM20A | 0.899 | 0.891 | 0.908 | 0.921 | 0.861 | 0.981 | 0.937 | 0.870 | 1 |
| IFI27_IFI44L_HERC6_IL1R2_OLAH | 0.899 | 0.891 | 0.908 | 0.927 | 0.878 | 0.976 | 0.937 | 0.880 | 0.993 |
| IFI27_IFI44L_HERC6_OLAH_MMP8 | 0.899 | 0.891 | 0.908 | 0.923 | 0.869 | 0.976 | 0.935 | 0.876 | 0.994 |
| IFI27_IFIT1_HERC6_OLAH_RETN | 0.899 | 0.891 | 0.908 | 0.920 | 0.863 | 0.977 | 0.942 | 0.885 | 1.000 |
| IFI27_OAS1_SIGLEC1_HERC6_OLAH | 0.899 | 0.891 | 0.907 | 0.922 | 0.869 | 0.975 | 0.940 | 0.885 | 0.996 |
| IFI27_OAS1_SIGLEC1_HERC6_IL1R2 | 0.899 | 0.892 | 0.907 | 0.922 | 0.876 | 0.969 | 0.949 | 0.902 | 0.996 |
| RSAD2_IFI27_HERC6_FAM20A_RETN | 0.899 | 0.890 | 0.908 | 0.922 | 0.864 | 0.979 | 0.934 | 0.854 | 1 |
| RSAD2_IFI27_IFI44L_OLAH_FAM20A | 0.899 | 0.890 | 0.908 | 0.923 | 0.863 | 0.983 | 0.934 | 0.864 | 1 |
| IFI27_OAS1_IFIT1_HERC6_OLAH | 0.899 | 0.891 | 0.907 | 0.924 | 0.871 | 0.976 | 0.935 | 0.876 | 0.994 |
| RSAD2_IFI27_IFIT1_OLAH_FAM20A | 0.899 | 0.890 | 0.907 | 0.920 | 0.860 | 0.980 | 0.937 | 0.870 | 1 |
| RSAD2_IFI27_OAS1_HERC6_OLAH | 0.899 | 0.891 | 0.907 | 0.923 | 0.872 | 0.975 | 0.933 | 0.873 | 0.992 |
| IFI27_OAS1_IFIT1_IL1R2_FAM20A | 0.899 | 0.891 | 0.907 | 0.923 | 0.870 | 0.977 | 0.929 | 0.850 | 1 |
| RSAD2_IFI27_IFIT1_IL1R2_FAM20A | 0.899 | 0.891 | 0.907 | 0.923 | 0.869 | 0.977 | 0.929 | 0.850 | 1 |
| IFIT1_SIGLEC1_IL1R2_FAM20A_MMP8 | 0.899 | 0.891 | 0.907 | 0.921 | 0.864 | 0.977 | 0.965 | 0.913 | 1 |
| IFI27_OAS1_IFI44L_IL1R2_FAM20A | 0.899 | 0.890 | 0.907 | 0.923 | 0.869 | 0.977 | 0.930 | 0.851 | 1 |
| IFI27_IFIT1_HERC6_OLAH_MMP8 | 0.899 | 0.890 | 0.907 | 0.923 | 0.870 | 0.976 | 0.939 | 0.882 | 0.996 |
| IFI27_IFI44L_SIGLEC1_IL1R2_OLAH | 0.899 | 0.891 | 0.906 | 0.923 | 0.872 | 0.974 | 0.947 | 0.896 | 0.997 |
| IFI27_HERC6_OLAH_RETN_MMP8 | 0.899 | 0.890 | 0.908 | 0.919 | 0.858 | 0.979 | 0.958 | 0.910 | 1 |
| IFI27_IFIT1_HERC6_FAM20A_RETN | 0.899 | 0.890 | 0.908 | 0.922 | 0.864 | 0.980 | 0.933 | 0.851 | 1 |
| IFI27_IFI44L_OLAH_FAM20A_RETN | 0.899 | 0.890 | 0.907 | 0.919 | 0.857 | 0.980 | 0.936 | 0.866 | 1 |
| IFI27_IFI44L_ISG15_HERC6_OLAH | 0.899 | 0.890 | 0.907 | 0.924 | 0.872 | 0.976 | 0.934 | 0.874 | 0.993 |
| IFI27_IFI44L_HERC6_OLAH_RETN | 0.899 | 0.890 | 0.907 | 0.921 | 0.865 | 0.977 | 0.945 | 0.890 | 0.999 |
| RSAD2_IFI27_IFI44L_IL1R2_FAM20A | 0.899 | 0.890 | 0.907 | 0.923 | 0.869 | 0.978 | 0.929 | 0.849 | 1 |
| IFI27_IFIT1_OLAH_FAM20A_RETN | 0.899 | 0.890 | 0.907 | 0.918 | 0.856 | 0.979 | 0.936 | 0.866 | 1 |
| IFI27_IFIT1_SIGLEC1_HERC6_OLAH | 0.899 | 0.890 | 0.907 | 0.922 | 0.868 | 0.976 | 0.943 | 0.892 | 0.995 |
| IFI27_OAS1_OLAH_FAM20A_RETN | 0.898 | 0.890 | 0.907 | 0.918 | 0.856 | 0.979 | 0.937 | 0.869 | 1 |
| IFI27_SIGLEC1_ISG15_SLC1A2_OLAH | 0.898 | 0.890 | 0.906 | 0.929 | 0.878 | 0.980 | 0.939 | 0.879 | 0.999 |
| IFI27_ISG15_IL1R2_OLAH_MMP8 | 0.898 | 0.891 | 0.906 | 0.922 | 0.871 | 0.973 | 0.937 | 0.878 | 0.996 |
| RSAD2_IFI27_HERC6_OLAH_RETN | 0.898 | 0.890 | 0.907 | 0.922 | 0.867 | 0.977 | 0.939 | 0.879 | 0.999 |
| IFI27_SIGLEC1_SLC1A2_OLAH_FAM20A | 0.898 | 0.889 | 0.907 | 0.925 | 0.867 | 0.983 | 0.945 | 0.877 | 1 |
| IFI27_OAS1_SIGLEC1_FAM20A_RETN | 0.898 | 0.889 | 0.907 | 0.917 | 0.855 | 0.979 | 0.943 | 0.872 | 1 |
| IFI27_HERC6_SLC1A2_OLAH_FAM20A | 0.898 | 0.889 | 0.907 | 0.926 | 0.871 | 0.981 | 0.926 | 0.847 | 1 |
| IFIT1_SIGLEC1_HERC6_IL1R2_FAM20A | 0.898 | 0.890 | 0.907 | 0.919 | 0.862 | 0.976 | 0.953 | 0.897 | 1 |
| RSAD2_IFI27_SIGLEC1_HERC6_FAM20A | 0.898 | 0.889 | 0.907 | 0.918 | 0.858 | 0.978 | 0.941 | 0.869 | 1 |
| IFI27_ISG15_HERC6_IL1R2_OLAH | 0.898 | 0.890 | 0.906 | 0.924 | 0.874 | 0.975 | 0.942 | 0.888 | 0.997 |
| IFI27_SIGLEC1_IL1R2_OLAH_MMP8 | 0.898 | 0.890 | 0.906 | 0.922 | 0.871 | 0.973 | 0.951 | 0.904 | 0.999 |
| IFI27_ISG15_HERC6_OLAH_MMP8 | 0.898 | 0.890 | 0.906 | 0.919 | 0.864 | 0.975 | 0.937 | 0.879 | 0.995 |
| RSAD2_IFI27_IFIT1_HERC6_OLAH | 0.898 | 0.890 | 0.906 | 0.922 | 0.870 | 0.975 | 0.936 | 0.878 | 0.994 |
| IFI27_OAS1_ISG15_HERC6_RETN | 0.898 | 0.889 | 0.907 | 0.917 | 0.858 | 0.976 | 0.932 | 0.863 | 1.000 |
| IFI27_SIGLEC1_ISG15_IL1R2_OLAH | 0.898 | 0.890 | 0.905 | 0.921 | 0.869 | 0.973 | 0.945 | 0.892 | 0.998 |
| IFI27_OAS1_IFI44L_HERC6_OLAH | 0.898 | 0.889 | 0.906 | 0.926 | 0.875 | 0.977 | 0.937 | 0.879 | 0.995 |
| IFI27_ISG15_OLAH_RETN_MMP8 | 0.898 | 0.889 | 0.907 | 0.920 | 0.861 | 0.978 | 0.948 | 0.889 | 1 |
| RSAD2_IFI27_SLC1A2_OLAH_FAM20A | 0.898 | 0.889 | 0.906 | 0.923 | 0.867 | 0.980 | 0.921 | 0.839 | 1 |
| IFI27_SIGLEC1_HERC6_IL1R2_OLAH | 0.898 | 0.890 | 0.906 | 0.924 | 0.873 | 0.975 | 0.945 | 0.892 | 0.997 |
| RSAD2_IFI27_OAS1_IL1R2_FAM20A | 0.898 | 0.889 | 0.906 | 0.925 | 0.873 | 0.978 | 0.929 | 0.848 | 1 |
| IFI27_ISG15_IL1R2_FAM20A_RETN | 0.898 | 0.890 | 0.906 | 0.923 | 0.871 | 0.976 | 0.916 | 0.829 | 1 |
| IFI27_IFI44L_SLC1A2_OLAH_FAM20A | 0.898 | 0.889 | 0.907 | 0.924 | 0.867 | 0.982 | 0.926 | 0.850 | 1 |
| RSAD2_IFI27_OLAH_FAM20A_RETN | 0.898 | 0.889 | 0.906 | 0.918 | 0.858 | 0.979 | 0.937 | 0.869 | 1 |
| IFI27_OAS1_HERC6_IL1R2_RETN | 0.898 | 0.890 | 0.906 | 0.922 | 0.869 | 0.974 | 0.938 | 0.878 | 0.998 |
| IFI44L_SIGLEC1_IL1R2_FAM20A_MMP8 | 0.898 | 0.889 | 0.906 | 0.919 | 0.861 | 0.976 | 0.961 | 0.903 | 1 |
| IFI27_HERC6_IL1R2_OLAH_MMP8 | 0.898 | 0.890 | 0.906 | 0.924 | 0.873 | 0.974 | 0.943 | 0.890 | 0.997 |
| IFI27_IFIT1_ISG15_IL1R2_FAM20A | 0.898 | 0.889 | 0.906 | 0.924 | 0.871 | 0.978 | 0.928 | 0.848 | 1 |
| IFI27_IFIT1_HERC6_IL1R2_OLAH | 0.898 | 0.889 | 0.906 | 0.925 | 0.875 | 0.975 | 0.942 | 0.888 | 0.997 |
| IFI27_IFI44L_SIGLEC1_HERC6_IL1R2 | 0.898 | 0.890 | 0.905 | 0.922 | 0.875 | 0.970 | 0.937 | 0.883 | 0.991 |
| IFI27_OAS1_ISG15_HERC6_OLAH | 0.897 | 0.889 | 0.906 | 0.922 | 0.870 | 0.975 | 0.936 | 0.877 | 0.995 |
| IFI27_SIGLEC1_SLC1A2_IL1R2_FAM20A | 0.897 | 0.889 | 0.906 | 0.928 | 0.877 | 0.979 | 0.947 | 0.875 | 1 |
| IFI27_IFI44L_SIGLEC1_ISG15_OLAH | 0.897 | 0.890 | 0.905 | 0.919 | 0.863 | 0.974 | 0.941 | 0.884 | 0.999 |
| IFI27_IFIT1_IL1R2_FAM20A_RETN | 0.897 | 0.889 | 0.906 | 0.924 | 0.871 | 0.978 | 0.929 | 0.850 | 1 |
| IFI27_IL1R2_OLAH_RETN_MMP8 | 0.897 | 0.889 | 0.906 | 0.923 | 0.868 | 0.977 | 0.948 | 0.890 | 1 |
| IFI27_ISG15_HERC6_OLAH_RETN | 0.897 | 0.889 | 0.906 | 0.918 | 0.860 | 0.976 | 0.943 | 0.887 | 1.000 |
| IFI27_IFI44L_SIGLEC1_OLAH_MMP8 | 0.897 | 0.889 | 0.905 | 0.920 | 0.864 | 0.976 | 0.949 | 0.898 | 1.000 |
| IFI27_IFI44L_IL1R2_OLAH_MMP8 | 0.897 | 0.889 | 0.905 | 0.922 | 0.871 | 0.974 | 0.940 | 0.884 | 0.996 |
| IFI27_IFIT1_ISG15_HERC6_OLAH | 0.897 | 0.889 | 0.905 | 0.920 | 0.865 | 0.975 | 0.938 | 0.880 | 0.996 |
| IFI27_OAS1_ISG15_IL1R2_FAM20A | 0.897 | 0.889 | 0.905 | 0.925 | 0.873 | 0.978 | 0.928 | 0.848 | 1 |
| IFI27_OAS1_IL1R2_FAM20A_RETN | 0.897 | 0.889 | 0.905 | 0.927 | 0.874 | 0.979 | 0.928 | 0.848 | 1 |
| IFI27_SIGLEC1_ISG15_HERC6_OLAH | 0.897 | 0.889 | 0.906 | 0.919 | 0.862 | 0.975 | 0.941 | 0.886 | 0.996 |
| RSAD2_IFI27_IFI44L_HERC6_OLAH | 0.897 | 0.889 | 0.905 | 0.926 | 0.874 | 0.977 | 0.938 | 0.882 | 0.995 |
| IFI27_IFI44L_HERC6_FAM20A_RETN | 0.897 | 0.888 | 0.906 | 0.920 | 0.861 | 0.979 | 0.938 | 0.860 | 1 |
| IFI27_IFIT1_IFI44L_SIGLEC1_FAM20A | 0.897 | 0.888 | 0.906 | 0.914 | 0.848 | 0.979 | 0.946 | 0.875 | 1 |
| IFI27_IFI44L_SIGLEC1_FAM20A_RETN | 0.897 | 0.888 | 0.906 | 0.919 | 0.860 | 0.979 | 0.946 | 0.873 | 1 |
| IFI27_IFIT1_SIGLEC1_ISG15_FAM20A | 0.897 | 0.889 | 0.905 | 0.916 | 0.853 | 0.980 | 0.942 | 0.870 | 1 |
| RSAD2_IFI27_SIGLEC1_FAM20A_RETN | 0.897 | 0.888 | 0.906 | 0.918 | 0.858 | 0.979 | 0.949 | 0.882 | 1 |
| IFI27_ISG15_HERC6_FAM20A_RETN | 0.897 | 0.888 | 0.906 | 0.920 | 0.861 | 0.979 | 0.941 | 0.864 | 1 |
| IFI27_OAS1_SIGLEC1_HERC6_FAM20A | 0.897 | 0.888 | 0.906 | 0.915 | 0.854 | 0.976 | 0.945 | 0.874 | 1 |
| IFI27_IL1R2_FAM20A_RETN_MMP8 | 0.897 | 0.888 | 0.905 | 0.924 | 0.873 | 0.975 | 0.911 | 0.819 | 1 |
| RSAD2_IFI27_IL1R2_FAM20A_RETN | 0.897 | 0.889 | 0.905 | 0.924 | 0.872 | 0.977 | 0.929 | 0.850 | 1 |
| IFI27_IFIT1_SLC1A2_OLAH_FAM20A | 0.897 | 0.888 | 0.906 | 0.922 | 0.865 | 0.980 | 0.925 | 0.847 | 1 |
| IFI27_SIGLEC1_HERC6_OLAH_MMP8 | 0.897 | 0.888 | 0.905 | 0.918 | 0.860 | 0.975 | 0.941 | 0.886 | 0.997 |
| IFI27_SLC1A2_OLAH_FAM20A_RETN | 0.897 | 0.888 | 0.906 | 0.925 | 0.869 | 0.980 | 0.921 | 0.839 | 1 |
| SIGLEC1_HERC6_IL1R2_FAM20A_MMP8 | 0.897 | 0.888 | 0.905 | 0.917 | 0.858 | 0.976 | 0.961 | 0.900 | 1 |
| RSAD2_IFI27_SIGLEC1_HERC6_IL1R2 | 0.897 | 0.889 | 0.904 | 0.920 | 0.872 | 0.968 | 0.949 | 0.902 | 0.996 |
| IFI27_IFIT1_SIGLEC1_IL1R2_OLAH | 0.897 | 0.889 | 0.904 | 0.920 | 0.868 | 0.973 | 0.941 | 0.887 | 0.996 |
| IFI27_IFIT1_IFI44L_HERC6_OLAH | 0.897 | 0.888 | 0.905 | 0.925 | 0.872 | 0.978 | 0.938 | 0.881 | 0.995 |
| RSAD2_IFI27_ISG15_HERC6_OLAH | 0.897 | 0.888 | 0.905 | 0.924 | 0.872 | 0.975 | 0.936 | 0.877 | 0.995 |
| IFI27_SIGLEC1_ISG15_OLAH_MMP8 | 0.897 | 0.888 | 0.905 | 0.916 | 0.859 | 0.974 | 0.942 | 0.886 | 0.999 |
| RSAD2_SIGLEC1_IL1R2_FAM20A_MMP8 | 0.897 | 0.888 | 0.905 | 0.919 | 0.862 | 0.976 | 0.963 | 0.908 | 1 |
| IFI27_IFI44L_SIGLEC1_SLC1A2_OLAH | 0.896 | 0.888 | 0.905 | 0.927 | 0.876 | 0.978 | 0.946 | 0.890 | 1 |
| RSAD2_IFI27_ISG15_IL1R2_FAM20A | 0.896 | 0.888 | 0.905 | 0.924 | 0.871 | 0.977 | 0.926 | 0.843 | 1 |
| IFI27_OAS1_SLC1A2_OLAH_FAM20A | 0.896 | 0.888 | 0.905 | 0.924 | 0.868 | 0.981 | 0.922 | 0.842 | 1 |
| SIGLEC1_IL1R2_OLAH_FAM20A_MMP8 | 0.896 | 0.888 | 0.905 | 0.918 | 0.859 | 0.976 | 0.964 | 0.909 | 1 |
| IFI27_OAS1_IFIT1_HERC6_RETN | 0.896 | 0.887 | 0.905 | 0.915 | 0.853 | 0.976 | 0.937 | 0.867 | 1 |
| RSAD2_IFI27_OAS1_HERC6_RETN | 0.896 | 0.887 | 0.905 | 0.915 | 0.853 | 0.977 | 0.933 | 0.858 | 1 |
| IFI27_OAS1_IFI44L_HERC6_RETN | 0.896 | 0.887 | 0.905 | 0.915 | 0.854 | 0.976 | 0.936 | 0.867 | 1 |
| IFI27_IFI44L_SIGLEC1_OLAH_RETN | 0.896 | 0.888 | 0.904 | 0.916 | 0.857 | 0.975 | 0.949 | 0.890 | 1 |
| RSAD2_IFI27_SIGLEC1_IL1R2_OLAH | 0.896 | 0.888 | 0.904 | 0.922 | 0.870 | 0.974 | 0.942 | 0.888 | 0.996 |
| IFI27_SIGLEC1_SLC1A2_OLAH_MMP8 | 0.896 | 0.888 | 0.905 | 0.927 | 0.876 | 0.979 | 0.941 | 0.883 | 1 |
| IFI27_OAS1_SIGLEC1_IL1R2_OLAH | 0.896 | 0.888 | 0.904 | 0.920 | 0.868 | 0.973 | 0.943 | 0.890 | 0.997 |
| IFI27_OAS1_HERC6_SLC1A2_OLAH | 0.896 | 0.887 | 0.905 | 0.928 | 0.879 | 0.978 | 0.933 | 0.868 | 0.997 |
| IFI27_IFI44L_OLAH_RETN_MMP8 | 0.896 | 0.887 | 0.905 | 0.920 | 0.861 | 0.979 | 0.952 | 0.897 | 1 |
| IFI27_IFIT1_IL1R2_OLAH_MMP8 | 0.896 | 0.888 | 0.904 | 0.922 | 0.870 | 0.973 | 0.945 | 0.892 | 0.997 |
| IFI27_IFI44L_SLC1A2_IL1R2_FAM20A | 0.896 | 0.888 | 0.904 | 0.927 | 0.878 | 0.976 | 0.927 | 0.843 | 1 |
| RSAD2_IFI27_SIGLEC1_HERC6_RETN | 0.896 | 0.887 | 0.905 | 0.914 | 0.855 | 0.973 | 0.949 | 0.897 | 1 |
| IFI27_OAS1_SIGLEC1_SLC1A2_OLAH | 0.896 | 0.888 | 0.904 | 0.927 | 0.875 | 0.979 | 0.945 | 0.888 | 1 |
| IFI27_SLC1A2_IL1R2_OLAH_MMP8 | 0.896 | 0.888 | 0.904 | 0.927 | 0.877 | 0.976 | 0.928 | 0.860 | 0.996 |
| IFI27_OAS1_IL1R2_OLAH_MMP8 | 0.896 | 0.888 | 0.904 | 0.921 | 0.869 | 0.973 | 0.943 | 0.890 | 0.997 |
| IFI27_IFIT1_SIGLEC1_OLAH_MMP8 | 0.896 | 0.888 | 0.904 | 0.918 | 0.862 | 0.974 | 0.941 | 0.885 | 0.997 |
| IFI27_SIGLEC1_OLAH_RETN_MMP8 | 0.896 | 0.887 | 0.905 | 0.918 | 0.857 | 0.978 | 0.963 | 0.914 | 1 |
| OAS1_SIGLEC1_IL1R2_FAM20A_MMP8 | 0.896 | 0.887 | 0.904 | 0.916 | 0.858 | 0.974 | 0.964 | 0.910 | 1 |
| RSAD2_IFI27_IL1R2_OLAH_MMP8 | 0.896 | 0.888 | 0.904 | 0.921 | 0.869 | 0.973 | 0.943 | 0.890 | 0.996 |
| IFI27_IFIT1_SIGLEC1_SLC1A2_OLAH | 0.896 | 0.887 | 0.904 | 0.927 | 0.876 | 0.979 | 0.942 | 0.884 | 1 |
| IFI27_IFI44L_HERC6_IL1R2_RETN | 0.896 | 0.888 | 0.904 | 0.919 | 0.866 | 0.973 | 0.934 | 0.872 | 0.995 |
| IFI27_IFIT1_ISG15_SLC1A2_OLAH | 0.896 | 0.887 | 0.904 | 0.926 | 0.874 | 0.978 | 0.928 | 0.861 | 0.996 |
| IFI27_IFI44L_SIGLEC1_IL1R2_RETN | 0.896 | 0.888 | 0.903 | 0.921 | 0.868 | 0.973 | 0.951 | 0.901 | 1 |
| IFI27_OAS1_IFIT1_SIGLEC1_OLAH | 0.896 | 0.888 | 0.904 | 0.919 | 0.864 | 0.974 | 0.941 | 0.885 | 0.997 |
| IFI27_ISG15_SLC1A2_OLAH_MMP8 | 0.896 | 0.887 | 0.904 | 0.926 | 0.874 | 0.977 | 0.924 | 0.852 | 0.996 |
| IFI27_IFIT1_OLAH_RETN_MMP8 | 0.896 | 0.887 | 0.905 | 0.918 | 0.857 | 0.978 | 0.952 | 0.896 | 1 |
| SIGLEC1_ISG15_IL1R2_FAM20A_MMP8 | 0.896 | 0.887 | 0.904 | 0.915 | 0.855 | 0.974 | 0.961 | 0.900 | 1 |
| IFI27_SIGLEC1_ISG15_FAM20A_RETN | 0.895 | 0.886 | 0.905 | 0.911 | 0.847 | 0.976 | 0.949 | 0.876 | 1 |
| IFI27_IFI44L_SIGLEC1_IL1R2_MMP8 | 0.895 | 0.888 | 0.903 | 0.919 | 0.870 | 0.967 | 0.945 | 0.895 | 0.994 |
| RSAD2_IFI27_SIGLEC1_SLC1A2_OLAH | 0.895 | 0.887 | 0.904 | 0.928 | 0.877 | 0.979 | 0.942 | 0.884 | 1 |
| IFI27_ISG15_IL1R2_OLAH_RETN | 0.895 | 0.887 | 0.904 | 0.919 | 0.864 | 0.975 | 0.940 | 0.878 | 1 |
| IFI27_IFIT1_SLC1A2_OLAH_MMP8 | 0.895 | 0.887 | 0.904 | 0.927 | 0.874 | 0.979 | 0.935 | 0.871 | 0.998 |
| IFI27_HERC6_IL1R2_OLAH_RETN | 0.895 | 0.887 | 0.904 | 0.918 | 0.860 | 0.975 | 0.947 | 0.892 | 1 |
| IFI27_IFI44L_SLC1A2_OLAH_MMP8 | 0.895 | 0.887 | 0.904 | 0.927 | 0.876 | 0.978 | 0.932 | 0.866 | 0.997 |
| IFI27_SIGLEC1_HERC6_OLAH_RETN | 0.895 | 0.887 | 0.904 | 0.918 | 0.858 | 0.977 | 0.948 | 0.893 | 1 |
| RSAD2_IFI27_HERC6_SLC1A2_OLAH | 0.895 | 0.887 | 0.904 | 0.927 | 0.877 | 0.978 | 0.927 | 0.860 | 0.994 |
| IFI27_IFIT1_SIGLEC1_OLAH_RETN | 0.895 | 0.887 | 0.904 | 0.916 | 0.858 | 0.975 | 0.950 | 0.893 | 1 |
| IFI27_IFI44L_ISG15_IL1R2_OLAH | 0.895 | 0.887 | 0.903 | 0.921 | 0.869 | 0.974 | 0.939 | 0.881 | 0.997 |
| IFI27_SIGLEC1_HERC6_FAM20A_RETN | 0.895 | 0.886 | 0.905 | 0.917 | 0.855 | 0.979 | 0.950 | 0.874 | 1 |
| IFI27_IFI44L_ISG15_OLAH_MMP8 | 0.895 | 0.887 | 0.903 | 0.917 | 0.859 | 0.974 | 0.934 | 0.871 | 0.996 |
| IFI27_ISG15_SLC1A2_IL1R2_OLAH | 0.895 | 0.887 | 0.903 | 0.928 | 0.879 | 0.977 | 0.924 | 0.852 | 0.996 |
| IFI27_ISG15_HERC6_SLC1A2_OLAH | 0.895 | 0.887 | 0.904 | 0.928 | 0.878 | 0.978 | 0.924 | 0.855 | 0.993 |
| RSAD2_IFI27_HERC6_IL1R2_RETN | 0.895 | 0.887 | 0.903 | 0.920 | 0.867 | 0.973 | 0.939 | 0.882 | 0.996 |
| IFI27_OAS1_OLAH_RETN_MMP8 | 0.895 | 0.886 | 0.904 | 0.919 | 0.860 | 0.979 | 0.951 | 0.894 | 1 |
| IFI27_IFI44L_ISG15_SLC1A2_OLAH | 0.895 | 0.887 | 0.903 | 0.926 | 0.875 | 0.978 | 0.925 | 0.855 | 0.995 |
| IFI27_IFIT1_SIGLEC1_HERC6_RETN | 0.895 | 0.886 | 0.904 | 0.912 | 0.850 | 0.973 | 0.947 | 0.894 | 0.999 |
| IFI27_IFIT1_SIGLEC1_HERC6_IL1R2 | 0.895 | 0.887 | 0.902 | 0.922 | 0.875 | 0.969 | 0.948 | 0.900 | 0.996 |
| IFI27_OAS1_IFIT1_IL1R2_OLAH | 0.895 | 0.887 | 0.903 | 0.920 | 0.868 | 0.973 | 0.941 | 0.885 | 0.997 |
| IFI27_SIGLEC1_IL1R2_RETN_MMP8 | 0.895 | 0.887 | 0.903 | 0.920 | 0.865 | 0.974 | 0.964 | 0.925 | 1 |
| IFI27_IFI44L_SLC1A2_IL1R2_OLAH | 0.895 | 0.886 | 0.903 | 0.927 | 0.877 | 0.977 | 0.927 | 0.858 | 0.996 |
| IFI27_IFIT1_SIGLEC1_FAM20A_MMP8 | 0.895 | 0.886 | 0.904 | 0.914 | 0.849 | 0.978 | 0.947 | 0.878 | 1 |
| IFI27_OAS1_SIGLEC1_OLAH_MMP8 | 0.895 | 0.886 | 0.903 | 0.918 | 0.862 | 0.974 | 0.945 | 0.890 | 0.999 |
| RSAD2_IFI27_SIGLEC1_OLAH_MMP8 | 0.895 | 0.886 | 0.903 | 0.919 | 0.863 | 0.975 | 0.942 | 0.887 | 0.998 |
| IFI27_IFIT1_SIGLEC1_ISG15_OLAH | 0.895 | 0.887 | 0.903 | 0.918 | 0.862 | 0.974 | 0.941 | 0.885 | 0.998 |
| RSAD2_IFI27_SLC1A2_OLAH_MMP8 | 0.895 | 0.886 | 0.903 | 0.926 | 0.875 | 0.978 | 0.932 | 0.866 | 0.997 |
| IFI27_SIGLEC1_ISG15_OLAH_RETN | 0.895 | 0.886 | 0.903 | 0.917 | 0.859 | 0.975 | 0.948 | 0.891 | 1 |
| IFI27_OAS1_SIGLEC1_IL1R2_RETN | 0.895 | 0.887 | 0.903 | 0.920 | 0.867 | 0.973 | 0.953 | 0.901 | 1 |
| IFIT1_SIGLEC1_HERC6_OLAH_FAM20A | 0.895 | 0.886 | 0.904 | 0.913 | 0.852 | 0.973 | 0.949 | 0.893 | 1 |
| IFI27_SIGLEC1_HERC6_SLC1A2_OLAH | 0.895 | 0.886 | 0.904 | 0.927 | 0.876 | 0.979 | 0.940 | 0.881 | 0.999 |
| IFI27_SIGLEC1_SLC1A2_IL1R2_OLAH | 0.895 | 0.886 | 0.903 | 0.927 | 0.877 | 0.977 | 0.942 | 0.886 | 0.998 |
| IFI27_OAS1_SLC1A2_OLAH_MMP8 | 0.895 | 0.886 | 0.903 | 0.926 | 0.875 | 0.978 | 0.929 | 0.862 | 0.997 |
| IFI27_OAS1_IFIT1_SIGLEC1_FAM20A | 0.894 | 0.886 | 0.903 | 0.913 | 0.848 | 0.978 | 0.948 | 0.880 | 1 |
| IFI27_IFIT1_IFI44L_SIGLEC1_OLAH | 0.894 | 0.886 | 0.903 | 0.919 | 0.863 | 0.976 | 0.949 | 0.898 | 1.000 |
| IFI27_HERC6_SLC1A2_IL1R2_FAM20A | 0.894 | 0.886 | 0.903 | 0.930 | 0.883 | 0.977 | 0.923 | 0.835 | 1 |
| IFI27_SIGLEC1_FAM20A_RETN_MMP8 | 0.894 | 0.885 | 0.904 | 0.916 | 0.852 | 0.979 | 0.954 | 0.882 | 1 |
| IFI27_OAS1_IFIT1_HERC6_IL1R2 | 0.894 | 0.887 | 0.902 | 0.917 | 0.868 | 0.966 | 0.939 | 0.884 | 0.994 |
| IFI27_IFIT1_ISG15_OLAH_MMP8 | 0.894 | 0.886 | 0.903 | 0.914 | 0.855 | 0.973 | 0.935 | 0.873 | 0.996 |
| IFI27_IFIT1_SIGLEC1_IL1R2_RETN | 0.894 | 0.886 | 0.902 | 0.920 | 0.866 | 0.973 | 0.959 | 0.915 | 1 |
| RSAD2_IFI27_OLAH_RETN_MMP8 | 0.894 | 0.885 | 0.903 | 0.918 | 0.858 | 0.978 | 0.951 | 0.894 | 1 |
| IFI27_IFI44L_SIGLEC1_ISG15_IL1R2 | 0.894 | 0.887 | 0.901 | 0.918 | 0.869 | 0.968 | 0.942 | 0.891 | 0.994 |
| IFI27_SLC1A2_OLAH_RETN_MMP8 | 0.894 | 0.885 | 0.903 | 0.926 | 0.873 | 0.978 | 0.941 | 0.878 | 1 |
| RSAD2_IFI27_IFIT1_IL1R2_OLAH | 0.894 | 0.886 | 0.902 | 0.921 | 0.869 | 0.974 | 0.941 | 0.885 | 0.997 |
| IFI27_OAS1_IFIT1_OLAH_MMP8 | 0.894 | 0.886 | 0.903 | 0.916 | 0.858 | 0.974 | 0.938 | 0.877 | 0.999 |
| IFI27_HERC6_SLC1A2_OLAH_MMP8 | 0.894 | 0.885 | 0.903 | 0.929 | 0.878 | 0.979 | 0.936 | 0.874 | 0.998 |
| IFI27_IFIT1_ISG15_IL1R2_OLAH | 0.894 | 0.886 | 0.902 | 0.919 | 0.865 | 0.972 | 0.941 | 0.885 | 0.998 |
| IFI27_OAS1_SIGLEC1_OLAH_RETN | 0.894 | 0.886 | 0.902 | 0.915 | 0.857 | 0.974 | 0.949 | 0.889 | 1 |
| RSAD2_IFI27_OAS1_IL1R2_OLAH | 0.894 | 0.886 | 0.902 | 0.921 | 0.868 | 0.974 | 0.941 | 0.886 | 0.997 |
| RSAD2_IFI27_ISG15_SLC1A2_OLAH | 0.894 | 0.886 | 0.902 | 0.927 | 0.875 | 0.979 | 0.928 | 0.860 | 0.996 |
| IFI27_SIGLEC1_IL1R2_OLAH_RETN | 0.894 | 0.886 | 0.902 | 0.917 | 0.860 | 0.975 | 0.950 | 0.896 | 1 |
| IFI27_OAS1_IFI44L_SIGLEC1_OLAH | 0.894 | 0.886 | 0.902 | 0.919 | 0.863 | 0.976 | 0.948 | 0.896 | 0.999 |
| RSAD2_IFI27_SLC1A2_IL1R2_OLAH | 0.894 | 0.885 | 0.902 | 0.926 | 0.876 | 0.976 | 0.933 | 0.867 | 0.998 |
| IFI27_IFIT1_HERC6_SLC1A2_OLAH | 0.894 | 0.885 | 0.903 | 0.929 | 0.879 | 0.980 | 0.936 | 0.874 | 0.997 |
| RSAD2_IFI27_IFIT1_SIGLEC1_FAM20A | 0.894 | 0.885 | 0.903 | 0.915 | 0.850 | 0.979 | 0.946 | 0.877 | 1 |
| IFI27_OAS1_SIGLEC1_ISG15_OLAH | 0.894 | 0.886 | 0.902 | 0.917 | 0.861 | 0.974 | 0.942 | 0.886 | 0.999 |
| IFI27_OAS1_IFIT1_SLC1A2_OLAH | 0.894 | 0.885 | 0.902 | 0.927 | 0.875 | 0.978 | 0.939 | 0.877 | 1 |
| IFI27_OAS1_SIGLEC1_IL1R2_MMP8 | 0.894 | 0.886 | 0.901 | 0.919 | 0.871 | 0.967 | 0.950 | 0.903 | 0.997 |
| IFI27_HERC6_SLC1A2_IL1R2_OLAH | 0.894 | 0.885 | 0.902 | 0.926 | 0.876 | 0.976 | 0.934 | 0.870 | 0.997 |
| IFI27_OAS1_IFIT1_OLAH_RETN | 0.894 | 0.885 | 0.902 | 0.915 | 0.855 | 0.975 | 0.943 | 0.881 | 1 |
| IFI27_IFIT1_SLC1A2_IL1R2_OLAH | 0.894 | 0.885 | 0.902 | 0.926 | 0.876 | 0.976 | 0.932 | 0.866 | 0.997 |
| SIGLEC1_IL1R2_FAM20A_RETN_MMP8 | 0.894 | 0.885 | 0.903 | 0.917 | 0.858 | 0.976 | 0.961 | 0.901 | 1 |
| IFI27_IFI44L_IL1R2_OLAH_RETN | 0.894 | 0.885 | 0.902 | 0.918 | 0.862 | 0.974 | 0.942 | 0.882 | 1 |
| RSAD2_IFI27_IFI44L_SIGLEC1_OLAH | 0.894 | 0.886 | 0.902 | 0.918 | 0.861 | 0.975 | 0.948 | 0.896 | 0.999 |
| IFI27_IFIT1_HERC6_IL1R2_RETN | 0.893 | 0.886 | 0.901 | 0.919 | 0.866 | 0.973 | 0.938 | 0.880 | 0.996 |
| IFI27_IFIT1_IFI44L_SIGLEC1_IL1R2 | 0.893 | 0.886 | 0.901 | 0.921 | 0.872 | 0.970 | 0.947 | 0.898 | 0.995 |
| RSAD2_IFI27_OAS1_SIGLEC1_OLAH | 0.893 | 0.885 | 0.902 | 0.919 | 0.863 | 0.974 | 0.941 | 0.885 | 0.998 |
| RSAD2_IFI27_SIGLEC1_ISG15_OLAH | 0.893 | 0.885 | 0.902 | 0.918 | 0.862 | 0.974 | 0.941 | 0.884 | 0.999 |
| IFIT1_SIGLEC1_HERC6_FAM20A_RETN | 0.893 | 0.885 | 0.902 | 0.914 | 0.853 | 0.975 | 0.940 | 0.873 | 1 |
| RSAD2_IFI27_IFIT1_SIGLEC1_OLAH | 0.893 | 0.885 | 0.902 | 0.919 | 0.863 | 0.975 | 0.940 | 0.884 | 0.997 |
| IFI27_SIGLEC1_HERC6_IL1R2_RETN | 0.893 | 0.885 | 0.902 | 0.922 | 0.869 | 0.975 | 0.951 | 0.903 | 0.999 |
| IFI27_HERC6_IL1R2_RETN_MMP8 | 0.893 | 0.885 | 0.902 | 0.922 | 0.868 | 0.975 | 0.939 | 0.881 | 0.998 |
| IFI27_OAS1_HERC6_IL1R2_MMP8 | 0.893 | 0.886 | 0.901 | 0.916 | 0.868 | 0.965 | 0.932 | 0.873 | 0.990 |
| RSAD2_IFI27_SIGLEC1_IL1R2_RETN | 0.893 | 0.885 | 0.902 | 0.921 | 0.869 | 0.974 | 0.957 | 0.911 | 1 |
| RSAD2_IFI27_SIGLEC1_OLAH_RETN | 0.893 | 0.885 | 0.902 | 0.916 | 0.857 | 0.975 | 0.947 | 0.889 | 1 |
| IFI27_HERC6_FAM20A_RETN_MMP8 | 0.893 | 0.883 | 0.903 | 0.920 | 0.861 | 0.980 | 0.940 | 0.862 | 1 |
| IFI27_IFIT1_IL1R2_OLAH_RETN | 0.893 | 0.885 | 0.902 | 0.918 | 0.861 | 0.974 | 0.943 | 0.885 | 1 |
| RSAD2_IFI27_SIGLEC1_IL1R2_MMP8 | 0.893 | 0.886 | 0.901 | 0.920 | 0.872 | 0.968 | 0.951 | 0.905 | 0.997 |
| RSAD2_IFI27_OAS1_OLAH_MMP8 | 0.893 | 0.885 | 0.902 | 0.916 | 0.859 | 0.974 | 0.937 | 0.877 | 0.997 |
| IFI27_IFI44L_ISG15_OLAH_RETN | 0.893 | 0.885 | 0.902 | 0.916 | 0.858 | 0.974 | 0.939 | 0.874 | 1 |
| RSAD2_IFI27_IL1R2_OLAH_RETN | 0.893 | 0.885 | 0.902 | 0.919 | 0.863 | 0.974 | 0.943 | 0.885 | 1 |
| IFI27_OAS1_IL1R2_OLAH_RETN | 0.893 | 0.885 | 0.901 | 0.918 | 0.862 | 0.974 | 0.943 | 0.885 | 1 |
| RSAD2_IFI27_IFIT1_SLC1A2_OLAH | 0.893 | 0.885 | 0.902 | 0.926 | 0.873 | 0.978 | 0.935 | 0.871 | 0.999 |
| IFI27_ISG15_SLC1A2_OLAH_RETN | 0.893 | 0.885 | 0.902 | 0.926 | 0.874 | 0.977 | 0.929 | 0.859 | 1.000 |
| IFI27_SIGLEC1_ISG15_IL1R2_MMP8 | 0.893 | 0.886 | 0.900 | 0.920 | 0.871 | 0.968 | 0.950 | 0.903 | 0.997 |
| RSAD2_IFI27_OAS1_HERC6_IL1R2 | 0.893 | 0.885 | 0.901 | 0.918 | 0.870 | 0.966 | 0.938 | 0.882 | 0.994 |
| IFI27_OAS1_ISG15_IL1R2_OLAH | 0.893 | 0.885 | 0.901 | 0.924 | 0.872 | 0.975 | 0.938 | 0.881 | 0.995 |
| IFI27_SLC1A2_IL1R2_OLAH_RETN | 0.893 | 0.885 | 0.902 | 0.926 | 0.874 | 0.977 | 0.929 | 0.858 | 1 |
| IFI27_OAS1_ISG15_OLAH_MMP8 | 0.893 | 0.885 | 0.901 | 0.916 | 0.858 | 0.974 | 0.936 | 0.877 | 0.995 |
| IFI27_SIGLEC1_HERC6_IL1R2_MMP8 | 0.893 | 0.886 | 0.901 | 0.920 | 0.872 | 0.969 | 0.949 | 0.902 | 0.996 |
| RSAD2_IFI27_OAS1_SLC1A2_OLAH | 0.893 | 0.884 | 0.902 | 0.926 | 0.873 | 0.978 | 0.935 | 0.869 | 1 |
| IFI27_IFIT1_IFI44L_OLAH_MMP8 | 0.893 | 0.884 | 0.902 | 0.917 | 0.859 | 0.975 | 0.940 | 0.882 | 0.998 |
| SIGLEC1_SLC1A2_IL1R2_FAM20A_MMP8 | 0.893 | 0.884 | 0.902 | 0.922 | 0.872 | 0.972 | 0.957 | 0.887 | 1 |
| IFI27_IFI44L_HERC6_IL1R2_MMP8 | 0.893 | 0.885 | 0.900 | 0.916 | 0.866 | 0.965 | 0.930 | 0.871 | 0.989 |
| IFI27_OAS1_IFIT1_SIGLEC1_IL1R2 | 0.893 | 0.885 | 0.901 | 0.920 | 0.871 | 0.969 | 0.951 | 0.904 | 0.998 |
| IFI27_OAS1_IFIT1_ISG15_OLAH | 0.893 | 0.885 | 0.901 | 0.914 | 0.854 | 0.973 | 0.935 | 0.873 | 0.996 |
| RSAD2_IFI27_ISG15_IL1R2_OLAH | 0.893 | 0.885 | 0.901 | 0.920 | 0.868 | 0.973 | 0.942 | 0.887 | 0.997 |
| RSAD2_IFI27_OAS1_SIGLEC1_FAM20A | 0.893 | 0.884 | 0.902 | 0.915 | 0.851 | 0.979 | 0.949 | 0.879 | 1 |
| IFI27_IFIT1_IFI44L_IL1R2_OLAH | 0.893 | 0.885 | 0.901 | 0.921 | 0.868 | 0.974 | 0.940 | 0.885 | 0.995 |
| IFI27_OAS1_SLC1A2_IL1R2_OLAH | 0.893 | 0.885 | 0.901 | 0.926 | 0.876 | 0.976 | 0.929 | 0.862 | 0.997 |
| IFI27_IFI44L_IL1R2_RETN_MMP8 | 0.893 | 0.885 | 0.901 | 0.919 | 0.866 | 0.972 | 0.940 | 0.879 | 1 |
| IFI27_SIGLEC1_SLC1A2_OLAH_RETN | 0.893 | 0.884 | 0.902 | 0.926 | 0.874 | 0.979 | 0.947 | 0.888 | 1 |
| IFI27_SIGLEC1_ISG15_IL1R2_RETN | 0.893 | 0.885 | 0.901 | 0.922 | 0.869 | 0.974 | 0.953 | 0.906 | 1 |
| RSAD2_IFI27_OAS1_SIGLEC1_IL1R2 | 0.893 | 0.885 | 0.901 | 0.920 | 0.871 | 0.968 | 0.958 | 0.914 | 1 |
| IFI27_ISG15_HERC6_IL1R2_RETN | 0.893 | 0.885 | 0.901 | 0.919 | 0.866 | 0.973 | 0.942 | 0.887 | 0.997 |
| IFI27_IFI44L_SIGLEC1_HERC6_RETN | 0.893 | 0.884 | 0.902 | 0.912 | 0.851 | 0.974 | 0.946 | 0.891 | 1 |
| IFI27_OAS1_ISG15_SLC1A2_OLAH | 0.893 | 0.884 | 0.901 | 0.927 | 0.875 | 0.978 | 0.921 | 0.849 | 0.993 |
| IFI27_IFIT1_ISG15_OLAH_RETN | 0.893 | 0.884 | 0.901 | 0.916 | 0.857 | 0.975 | 0.940 | 0.878 | 1 |
| RSAD2_IFI27_SLC1A2_IL1R2_FAM20A | 0.893 | 0.884 | 0.901 | 0.926 | 0.878 | 0.975 | 0.926 | 0.840 | 1 |
| RSAD2_IFI27_IFIT1_OLAH_MMP8 | 0.893 | 0.884 | 0.901 | 0.916 | 0.858 | 0.974 | 0.938 | 0.877 | 0.999 |
| IFI27_IFIT1_SIGLEC1_IL1R2_MMP8 | 0.893 | 0.885 | 0.900 | 0.919 | 0.870 | 0.968 | 0.951 | 0.905 | 0.997 |
| RSAD2_IFI27_IFIT1_HERC6_IL1R2 | 0.893 | 0.885 | 0.900 | 0.918 | 0.869 | 0.967 | 0.936 | 0.880 | 0.992 |
| IFI27_SIGLEC1_ISG15_HERC6_IL1R2 | 0.893 | 0.885 | 0.900 | 0.919 | 0.870 | 0.968 | 0.949 | 0.902 | 0.996 |
| RSAD2_IFI27_SIGLEC1_ISG15_FAM20A | 0.893 | 0.884 | 0.902 | 0.914 | 0.851 | 0.978 | 0.947 | 0.877 | 1 |
| IFI27_SLC1A2_IL1R2_FAM20A_MMP8 | 0.893 | 0.884 | 0.901 | 0.925 | 0.878 | 0.972 | 0.905 | 0.810 | 1 |
| IFI27_OAS1_IFI44L_SIGLEC1_IL1R2 | 0.892 | 0.885 | 0.900 | 0.919 | 0.869 | 0.968 | 0.942 | 0.891 | 0.994 |
| IFI27_IFI44L_SLC1A2_OLAH_RETN | 0.892 | 0.884 | 0.901 | 0.927 | 0.876 | 0.978 | 0.935 | 0.868 | 1 |
| IFI27_OAS1_IFI44L_IL1R2_OLAH | 0.892 | 0.884 | 0.901 | 0.922 | 0.869 | 0.974 | 0.939 | 0.883 | 0.995 |
| IFI27_HERC6_SLC1A2_OLAH_RETN | 0.892 | 0.883 | 0.901 | 0.925 | 0.872 | 0.978 | 0.940 | 0.878 | 1 |
| RSAD2_IFI27_IFI44L_IL1R2_OLAH | 0.892 | 0.884 | 0.900 | 0.921 | 0.867 | 0.974 | 0.941 | 0.886 | 0.997 |
| IFI27_IFIT1_SLC1A2_IL1R2_FAM20A | 0.892 | 0.884 | 0.901 | 0.924 | 0.873 | 0.975 | 0.927 | 0.842 | 1 |
| IFI27_OAS1_IFIT1_IL1R2_RETN | 0.892 | 0.884 | 0.900 | 0.918 | 0.864 | 0.971 | 0.949 | 0.896 | 1 |
| IFI27_OAS1_IFIT1_IFI44L_OLAH | 0.892 | 0.884 | 0.901 | 0.916 | 0.858 | 0.974 | 0.939 | 0.881 | 0.998 |
| IFI27_IFI44L_HERC6_SLC1A2_OLAH | 0.892 | 0.883 | 0.902 | 0.928 | 0.877 | 0.978 | 0.933 | 0.870 | 0.995 |
| RSAD2_IFI27_OAS1_IFIT1_OLAH | 0.892 | 0.884 | 0.900 | 0.916 | 0.858 | 0.974 | 0.938 | 0.879 | 0.997 |
| IFI27_IFIT1_SIGLEC1_ISG15_IL1R2 | 0.892 | 0.885 | 0.900 | 0.920 | 0.871 | 0.968 | 0.947 | 0.897 | 0.996 |
| IFI27_IFIT1_IFI44L_OLAH_RETN | 0.892 | 0.884 | 0.901 | 0.914 | 0.855 | 0.974 | 0.945 | 0.884 | 1 |
| IFI27_ISG15_IL1R2_RETN_MMP8 | 0.892 | 0.884 | 0.900 | 0.924 | 0.873 | 0.976 | 0.933 | 0.866 | 0.999 |
| IFI27_IFIT1_IFI44L_ISG15_OLAH | 0.892 | 0.884 | 0.900 | 0.915 | 0.857 | 0.974 | 0.936 | 0.876 | 0.996 |
| RSAD2_IFI27_IFI44L_SIGLEC1_IL1R2 | 0.892 | 0.884 | 0.900 | 0.920 | 0.870 | 0.970 | 0.946 | 0.897 | 0.995 |
| IFI27_OAS1_IFI44L_OLAH_MMP8 | 0.892 | 0.883 | 0.900 | 0.917 | 0.860 | 0.974 | 0.937 | 0.878 | 0.996 |
| IFI27_OAS1_IFI44L_HERC6_IL1R2 | 0.892 | 0.884 | 0.899 | 0.919 | 0.871 | 0.966 | 0.929 | 0.870 | 0.988 |
| RSAD2_IFI27_HERC6_IL1R2_MMP8 | 0.892 | 0.884 | 0.900 | 0.915 | 0.866 | 0.965 | 0.934 | 0.876 | 0.991 |
| RSAD2_IFI27_OAS1_ISG15_OLAH | 0.892 | 0.884 | 0.900 | 0.916 | 0.858 | 0.974 | 0.935 | 0.874 | 0.995 |
| RSAD2_IFI27_ISG15_OLAH_MMP8 | 0.892 | 0.883 | 0.900 | 0.915 | 0.856 | 0.974 | 0.935 | 0.874 | 0.996 |
| RSAD2_IFI27_OAS1_OLAH_RETN | 0.892 | 0.883 | 0.900 | 0.914 | 0.853 | 0.974 | 0.943 | 0.882 | 1 |
| IFI27_IFIT1_SLC1A2_OLAH_RETN | 0.892 | 0.883 | 0.900 | 0.925 | 0.874 | 0.977 | 0.938 | 0.873 | 1 |
| IFI27_IFIT1_IL1R2_RETN_MMP8 | 0.892 | 0.883 | 0.900 | 0.918 | 0.863 | 0.973 | 0.949 | 0.897 | 1 |
| IFI27_OAS1_SIGLEC1_ISG15_FAM20A | 0.892 | 0.882 | 0.901 | 0.911 | 0.846 | 0.976 | 0.946 | 0.875 | 1 |
| OAS1_IFIT1_SIGLEC1_IL1R2_FAM20A | 0.892 | 0.883 | 0.900 | 0.914 | 0.851 | 0.976 | 0.962 | 0.913 | 1 |
| IFI27_IFI44L_SIGLEC1_ISG15_FAM20A | 0.892 | 0.883 | 0.901 | 0.910 | 0.844 | 0.976 | 0.943 | 0.868 | 1 |
| IFI27_IFIT1_IFI44L_HERC6_IL1R2 | 0.892 | 0.884 | 0.899 | 0.918 | 0.868 | 0.969 | 0.938 | 0.883 | 0.993 |
| IFI27_OAS1_ISG15_HERC6_IL1R2 | 0.892 | 0.884 | 0.899 | 0.920 | 0.873 | 0.967 | 0.932 | 0.873 | 0.990 |
| RSAD2_IFI27_SLC1A2_OLAH_RETN | 0.892 | 0.883 | 0.900 | 0.925 | 0.873 | 0.977 | 0.934 | 0.866 | 1 |
| RSAD2_IFI27_IFI44L_SIGLEC1_FAM20A | 0.892 | 0.883 | 0.901 | 0.914 | 0.849 | 0.978 | 0.948 | 0.878 | 1 |
| IFIT1_SIGLEC1_ISG15_HERC6_FAM20A | 0.892 | 0.883 | 0.900 | 0.911 | 0.849 | 0.972 | 0.938 | 0.865 | 1 |
| IFI44L_SIGLEC1_HERC6_IL1R2_FAM20A | 0.892 | 0.883 | 0.900 | 0.913 | 0.852 | 0.974 | 0.957 | 0.896 | 1 |
| IFI27_OAS1_ISG15_OLAH_RETN | 0.892 | 0.883 | 0.900 | 0.916 | 0.858 | 0.975 | 0.938 | 0.875 | 1 |
| IFI27_OAS1_SLC1A2_OLAH_RETN | 0.892 | 0.883 | 0.900 | 0.925 | 0.874 | 0.977 | 0.935 | 0.868 | 1 |
| RSAD2_IFI27_IFIT1_ISG15_OLAH | 0.891 | 0.883 | 0.900 | 0.914 | 0.856 | 0.973 | 0.935 | 0.873 | 0.996 |
| IFI27_IFIT1_IFI44L_SLC1A2_OLAH | 0.891 | 0.883 | 0.900 | 0.925 | 0.873 | 0.978 | 0.936 | 0.873 | 0.999 |
| RSAD2_IFI27_IFI44L_HERC6_IL1R2 | 0.891 | 0.884 | 0.899 | 0.918 | 0.870 | 0.967 | 0.932 | 0.874 | 0.989 |
| IFI27_OAS1_IFI44L_ISG15_OLAH | 0.891 | 0.883 | 0.900 | 0.916 | 0.859 | 0.974 | 0.934 | 0.874 | 0.994 |
| RSAD2_IFI27_IFIT1_SIGLEC1_IL1R2 | 0.891 | 0.884 | 0.899 | 0.919 | 0.870 | 0.968 | 0.949 | 0.901 | 0.997 |
| IFI27_IFIT1_HERC6_IL1R2_MMP8 | 0.891 | 0.884 | 0.899 | 0.915 | 0.864 | 0.965 | 0.939 | 0.886 | 0.993 |
| RSAD2_IFI27_IFI44L_OLAH_MMP8 | 0.891 | 0.883 | 0.900 | 0.918 | 0.861 | 0.975 | 0.939 | 0.881 | 0.997 |
| RSAD2_IFI27_IFIT1_OLAH_RETN | 0.891 | 0.883 | 0.900 | 0.914 | 0.855 | 0.974 | 0.945 | 0.884 | 1 |
| IFI27_OAS1_IFI44L_OLAH_RETN | 0.891 | 0.883 | 0.900 | 0.915 | 0.856 | 0.974 | 0.942 | 0.882 | 1 |
| RSAD2_IFI27_IFI44L_SLC1A2_OLAH | 0.891 | 0.883 | 0.900 | 0.927 | 0.875 | 0.978 | 0.932 | 0.866 | 0.997 |
| IFI27_SLC1A2_IL1R2_FAM20A_RETN | 0.891 | 0.883 | 0.900 | 0.927 | 0.880 | 0.974 | 0.907 | 0.810 | 1 |
| IFI27_IFI44L_ISG15_HERC6_IL1R2 | 0.891 | 0.884 | 0.899 | 0.920 | 0.873 | 0.968 | 0.928 | 0.869 | 0.988 |
| IFI27_OAS1_SLC1A2_IL1R2_FAM20A | 0.891 | 0.883 | 0.900 | 0.928 | 0.880 | 0.977 | 0.925 | 0.839 | 1 |
| RSAD2_SIGLEC1_HERC6_IL1R2_FAM20A | 0.891 | 0.882 | 0.900 | 0.916 | 0.857 | 0.974 | 0.955 | 0.898 | 1 |
| IFI27_ISG15_SLC1A2_IL1R2_FAM20A | 0.891 | 0.883 | 0.899 | 0.923 | 0.873 | 0.973 | 0.910 | 0.816 | 1 |
| IFIT1_IFI44L_SIGLEC1_HERC6_FAM20A | 0.891 | 0.882 | 0.900 | 0.912 | 0.850 | 0.973 | 0.937 | 0.867 | 1 |
| IFI27_IFIT1_IFI44L_IL1R2_RETN | 0.891 | 0.883 | 0.899 | 0.920 | 0.866 | 0.973 | 0.941 | 0.883 | 0.999 |
| IFI27_OAS1_HERC6_RETN_MMP8 | 0.891 | 0.882 | 0.900 | 0.916 | 0.857 | 0.976 | 0.929 | 0.857 | 1 |
| RSAD2_IFI27_SIGLEC1_ISG15_IL1R2 | 0.891 | 0.883 | 0.899 | 0.920 | 0.872 | 0.969 | 0.948 | 0.899 | 0.997 |
| RSAD2_IFI27_OAS1_IFI44L_OLAH | 0.891 | 0.883 | 0.899 | 0.918 | 0.861 | 0.975 | 0.939 | 0.881 | 0.997 |
| IFI27_IFI44L_SIGLEC1_SLC1A2_IL1R2 | 0.891 | 0.883 | 0.899 | 0.924 | 0.878 | 0.970 | 0.947 | 0.897 | 0.996 |
| IFI27_OAS1_SIGLEC1_ISG15_IL1R2 | 0.891 | 0.883 | 0.898 | 0.919 | 0.870 | 0.968 | 0.950 | 0.903 | 0.997 |
| RSAD2_IFI27_IFIT1_IL1R2_RETN | 0.891 | 0.883 | 0.899 | 0.917 | 0.864 | 0.971 | 0.946 | 0.892 | 0.999 |
| RSAD2_IFI27_ISG15_OLAH_RETN | 0.891 | 0.882 | 0.899 | 0.916 | 0.857 | 0.975 | 0.939 | 0.876 | 1 |
| IFI27_OAS1_SIGLEC1_SLC1A2_IL1R2 | 0.891 | 0.883 | 0.899 | 0.924 | 0.878 | 0.970 | 0.950 | 0.901 | 0.999 |
| IFI27_OAS1_IFI44L_SLC1A2_OLAH | 0.891 | 0.882 | 0.899 | 0.926 | 0.875 | 0.978 | 0.935 | 0.871 | 0.999 |
| RSAD2_IFI27_SIGLEC1_FAM20A_MMP8 | 0.890 | 0.881 | 0.900 | 0.913 | 0.849 | 0.978 | 0.948 | 0.878 | 1 |
| RSAD2_IFI27_IFI44L_ISG15_OLAH | 0.890 | 0.882 | 0.899 | 0.916 | 0.858 | 0.975 | 0.937 | 0.878 | 0.996 |
| RSAD2_IFI27_IFIT1_IFI44L_OLAH | 0.890 | 0.882 | 0.899 | 0.916 | 0.858 | 0.974 | 0.939 | 0.881 | 0.998 |
| IFI27_SIGLEC1_SLC1A2_IL1R2_RETN | 0.890 | 0.882 | 0.898 | 0.921 | 0.871 | 0.971 | 0.957 | 0.910 | 1 |
| RSAD2_IFI27_ISG15_HERC6_IL1R2 | 0.890 | 0.883 | 0.898 | 0.917 | 0.868 | 0.966 | 0.929 | 0.870 | 0.988 |
| RSAD2_IFI27_IFI44L_OLAH_RETN | 0.890 | 0.882 | 0.899 | 0.915 | 0.856 | 0.974 | 0.942 | 0.881 | 1 |
| IFI27_OAS1_IFI44L_SIGLEC1_FAM20A | 0.890 | 0.881 | 0.899 | 0.910 | 0.844 | 0.975 | 0.947 | 0.876 | 1 |
| RSAD2_IFI27_IFI44L_HERC6_RETN | 0.890 | 0.881 | 0.899 | 0.913 | 0.853 | 0.974 | 0.940 | 0.880 | 1 |
| IFI27_OAS1_SIGLEC1_FAM20A_MMP8 | 0.890 | 0.881 | 0.900 | 0.911 | 0.847 | 0.976 | 0.948 | 0.879 | 1 |
| OAS1_SIGLEC1_HERC6_IL1R2_FAM20A | 0.890 | 0.881 | 0.899 | 0.913 | 0.853 | 0.972 | 0.959 | 0.905 | 1 |
| IFI27_SIGLEC1_ISG15_SLC1A2_IL1R2 | 0.890 | 0.883 | 0.897 | 0.924 | 0.878 | 0.969 | 0.949 | 0.900 | 0.998 |
| IFIT1_SIGLEC1_HERC6_FAM20A_MMP8 | 0.890 | 0.881 | 0.899 | 0.910 | 0.848 | 0.972 | 0.936 | 0.863 | 1 |
| RSAD2_IFI27_SIGLEC1_SLC1A2_IL1R2 | 0.890 | 0.882 | 0.898 | 0.924 | 0.879 | 0.970 | 0.953 | 0.906 | 1 |
| IFI27_IFI44L_SIGLEC1_HERC6_FAM20A | 0.890 | 0.881 | 0.899 | 0.911 | 0.848 | 0.974 | 0.947 | 0.870 | 1 |
| RSAD2_IFI27_ISG15_HERC6_RETN | 0.890 | 0.881 | 0.899 | 0.914 | 0.855 | 0.973 | 0.941 | 0.884 | 0.999 |
| IFIT1_IFI44L_SIGLEC1_IL1R2_FAM20A | 0.890 | 0.881 | 0.898 | 0.909 | 0.845 | 0.973 | 0.958 | 0.907 | 1 |
| IFI27_ISG15_HERC6_IL1R2_MMP8 | 0.890 | 0.882 | 0.897 | 0.916 | 0.866 | 0.965 | 0.933 | 0.875 | 0.990 |
| IFI27_IFI44L_ISG15_IL1R2_RETN | 0.890 | 0.882 | 0.898 | 0.920 | 0.867 | 0.972 | 0.942 | 0.886 | 0.999 |
| RSAD2_IFI27_OAS1_IL1R2_RETN | 0.890 | 0.882 | 0.898 | 0.918 | 0.864 | 0.972 | 0.943 | 0.886 | 1 |
| IFI27_OAS1_IFIT1_IL1R2_MMP8 | 0.890 | 0.882 | 0.897 | 0.916 | 0.865 | 0.966 | 0.943 | 0.891 | 0.996 |
| IFI27_IFIT1_ISG15_HERC6_IL1R2 | 0.890 | 0.882 | 0.897 | 0.915 | 0.865 | 0.965 | 0.938 | 0.884 | 0.992 |
| IFI27_OAS1_IL1R2_RETN_MMP8 | 0.890 | 0.881 | 0.898 | 0.918 | 0.864 | 0.972 | 0.940 | 0.881 | 0.999 |
| RSAD2_IFI27_IL1R2_RETN_MMP8 | 0.890 | 0.881 | 0.898 | 0.919 | 0.865 | 0.973 | 0.941 | 0.882 | 1 |
| IFI27_IFIT1_IFI44L_IL1R2_MMP8 | 0.889 | 0.882 | 0.897 | 0.917 | 0.865 | 0.968 | 0.937 | 0.881 | 0.993 |
| RSAD2_IFI27_IFIT1_HERC6_RETN | 0.889 | 0.880 | 0.898 | 0.912 | 0.852 | 0.973 | 0.941 | 0.881 | 1 |
| RSAD2_IFIT1_SIGLEC1_IL1R2_FAM20A | 0.889 | 0.881 | 0.898 | 0.911 | 0.847 | 0.974 | 0.963 | 0.914 | 1 |
| IFI27_IFIT1_SIGLEC1_SLC1A2_FAM20A | 0.889 | 0.881 | 0.898 | 0.919 | 0.862 | 0.976 | 0.946 | 0.871 | 1 |
| OAS1_IFIT1_SIGLEC1_HERC6_FAM20A | 0.889 | 0.880 | 0.898 | 0.910 | 0.849 | 0.971 | 0.938 | 0.868 | 1 |
| RSAD2_IFI27_OAS1_IFIT1_IL1R2 | 0.889 | 0.881 | 0.897 | 0.914 | 0.863 | 0.965 | 0.945 | 0.892 | 0.997 |
| RSAD2_IFI27_IFIT1_IL1R2_MMP8 | 0.889 | 0.881 | 0.897 | 0.916 | 0.865 | 0.966 | 0.942 | 0.890 | 0.995 |
| IFI27_OAS1_IFI44L_IL1R2_RETN | 0.889 | 0.881 | 0.897 | 0.918 | 0.865 | 0.971 | 0.939 | 0.881 | 0.997 |
| IFI27_SIGLEC1_ISG15_HERC6_FAM20A | 0.889 | 0.880 | 0.899 | 0.906 | 0.837 | 0.974 | 0.948 | 0.872 | 1 |
| SIGLEC1_ISG15_HERC6_IL1R2_FAM20A | 0.889 | 0.880 | 0.898 | 0.912 | 0.850 | 0.974 | 0.959 | 0.900 | 1 |
| IFI27_IFIT1_ISG15_IL1R2_RETN | 0.889 | 0.881 | 0.897 | 0.917 | 0.864 | 0.971 | 0.947 | 0.894 | 1.000 |
| IFI27_OAS1_IFIT1_IFI44L_IL1R2 | 0.889 | 0.881 | 0.897 | 0.916 | 0.864 | 0.968 | 0.938 | 0.883 | 0.993 |
| IFI27_IFI44L_SLC1A2_IL1R2_RETN | 0.889 | 0.881 | 0.897 | 0.924 | 0.875 | 0.972 | 0.933 | 0.866 | 0.999 |
| IFI27_OAS1_IFIT1_SIGLEC1_RETN | 0.889 | 0.880 | 0.898 | 0.909 | 0.847 | 0.970 | 0.954 | 0.896 | 1 |
| IFI27_IFIT1_SIGLEC1_SLC1A2_IL1R2 | 0.889 | 0.881 | 0.897 | 0.924 | 0.878 | 0.970 | 0.954 | 0.909 | 1.000 |
| IFIT1_SIGLEC1_IL1R2_OLAH_FAM20A | 0.889 | 0.880 | 0.898 | 0.913 | 0.851 | 0.975 | 0.962 | 0.914 | 1 |
| IFI27_SIGLEC1_SLC1A2_IL1R2_MMP8 | 0.889 | 0.881 | 0.897 | 0.924 | 0.878 | 0.970 | 0.955 | 0.911 | 1 |
| SIGLEC1_HERC6_IL1R2_OLAH_FAM20A | 0.889 | 0.880 | 0.898 | 0.911 | 0.848 | 0.974 | 0.959 | 0.900 | 1 |
| SIGLEC1_SLC1A2_OLAH_FAM20A_MMP8 | 0.889 | 0.879 | 0.898 | 0.919 | 0.863 | 0.974 | 0.951 | 0.888 | 1 |
| IFI27_IFI44L_ISG15_IL1R2_MMP8 | 0.889 | 0.881 | 0.896 | 0.915 | 0.865 | 0.966 | 0.937 | 0.880 | 0.994 |
| IFI27_IFIT1_IFI44L_ISG15_IL1R2 | 0.889 | 0.880 | 0.897 | 0.916 | 0.864 | 0.968 | 0.939 | 0.885 | 0.994 |
| OAS1_IFIT1_SIGLEC1_OLAH_FAM20A | 0.889 | 0.879 | 0.898 | 0.906 | 0.837 | 0.975 | 0.955 | 0.906 | 1 |
| RSAD2_IFIT1_SIGLEC1_HERC6_FAM20A | 0.889 | 0.880 | 0.897 | 0.911 | 0.849 | 0.972 | 0.937 | 0.865 | 1 |
| RSAD2_IFI27_IFIT1_IFI44L_IL1R2 | 0.888 | 0.881 | 0.896 | 0.915 | 0.863 | 0.968 | 0.938 | 0.883 | 0.993 |
| IFI27_IFI44L_SIGLEC1_FAM20A_MMP8 | 0.888 | 0.879 | 0.898 | 0.911 | 0.846 | 0.976 | 0.947 | 0.872 | 1 |
| IFI27_SIGLEC1_SLC1A2_FAM20A_RETN | 0.888 | 0.879 | 0.898 | 0.919 | 0.860 | 0.979 | 0.948 | 0.871 | 1 |
| IFIT1_SIGLEC1_OLAH_FAM20A_MMP8 | 0.888 | 0.879 | 0.898 | 0.906 | 0.841 | 0.972 | 0.955 | 0.907 | 1 |
| RSAD2_IFI27_IFI44L_IL1R2_RETN | 0.888 | 0.880 | 0.896 | 0.918 | 0.863 | 0.972 | 0.938 | 0.878 | 0.998 |
| IFI27_HERC6_SLC1A2_IL1R2_RETN | 0.888 | 0.879 | 0.896 | 0.923 | 0.873 | 0.973 | 0.935 | 0.871 | 0.999 |
| IFI27_SIGLEC1_ISG15_FAM20A_MMP8 | 0.888 | 0.878 | 0.898 | 0.905 | 0.837 | 0.974 | 0.943 | 0.868 | 1 |
| IFI44L_SIGLEC1_ISG15_IL1R2_FAM20A | 0.888 | 0.879 | 0.897 | 0.910 | 0.848 | 0.973 | 0.962 | 0.911 | 1 |
| IFI27_IFI44L_ISG15_FAM20A_RETN | 0.888 | 0.878 | 0.897 | 0.911 | 0.848 | 0.974 | 0.945 | 0.868 | 1 |
| IFI27_IFIT1_ISG15_HERC6_RETN | 0.888 | 0.878 | 0.897 | 0.910 | 0.848 | 0.971 | 0.943 | 0.882 | 1 |
| IFI27_SIGLEC1_HERC6_SLC1A2_IL1R2 | 0.888 | 0.880 | 0.896 | 0.924 | 0.878 | 0.970 | 0.957 | 0.912 | 1 |
| IFI44L_SIGLEC1_IL1R2_OLAH_FAM20A | 0.888 | 0.879 | 0.897 | 0.910 | 0.848 | 0.973 | 0.963 | 0.912 | 1 |
| IFI27_IFIT1_ISG15_IL1R2_MMP8 | 0.888 | 0.880 | 0.895 | 0.915 | 0.864 | 0.966 | 0.941 | 0.888 | 0.994 |
| OAS1_IFI44L_SIGLEC1_IL1R2_FAM20A | 0.887 | 0.879 | 0.896 | 0.910 | 0.846 | 0.973 | 0.964 | 0.914 | 1 |
| IFIT1_SIGLEC1_ISG15_IL1R2_FAM20A | 0.887 | 0.879 | 0.896 | 0.913 | 0.850 | 0.975 | 0.962 | 0.914 | 1 |
| RSAD2_IFI27_IFI44L_IL1R2_MMP8 | 0.887 | 0.880 | 0.895 | 0.915 | 0.863 | 0.966 | 0.936 | 0.880 | 0.992 |
| IFIT1_IFI44L_SIGLEC1_OLAH_FAM20A | 0.887 | 0.878 | 0.896 | 0.905 | 0.836 | 0.973 | 0.943 | 0.887 | 1 |
| IFI27_IFIT1_IFI44L_HERC6_RETN | 0.887 | 0.878 | 0.897 | 0.910 | 0.847 | 0.972 | 0.940 | 0.875 | 1 |
| IFIT1_SIGLEC1_IL1R2_FAM20A_RETN | 0.887 | 0.878 | 0.896 | 0.909 | 0.846 | 0.972 | 0.961 | 0.910 | 1 |
| RSAD2_IFI27_OAS1_IFI44L_IL1R2 | 0.887 | 0.880 | 0.895 | 0.914 | 0.863 | 0.966 | 0.940 | 0.886 | 0.994 |
| RSAD2_IFI27_OAS1_SIGLEC1_RETN | 0.887 | 0.878 | 0.896 | 0.909 | 0.847 | 0.971 | 0.957 | 0.897 | 1 |
| OAS1_SIGLEC1_IL1R2_OLAH_FAM20A | 0.887 | 0.878 | 0.896 | 0.910 | 0.847 | 0.973 | 0.962 | 0.910 | 1 |
| RSAD2_IFI27_OAS1_IL1R2_MMP8 | 0.887 | 0.879 | 0.895 | 0.915 | 0.865 | 0.966 | 0.939 | 0.884 | 0.994 |
| RSAD2_IFI27_ISG15_IL1R2_RETN | 0.887 | 0.879 | 0.895 | 0.918 | 0.865 | 0.972 | 0.940 | 0.882 | 0.998 |
| IFIT1_SIGLEC1_HERC6_SLC1A2_FAM20A | 0.887 | 0.878 | 0.896 | 0.918 | 0.864 | 0.971 | 0.938 | 0.862 | 1 |
| RSAD2_OAS1_SIGLEC1_IL1R2_FAM20A | 0.887 | 0.878 | 0.896 | 0.910 | 0.847 | 0.973 | 0.963 | 0.912 | 1 |
| IFI27_IFI44L_FAM20A_RETN_MMP8 | 0.887 | 0.877 | 0.897 | 0.914 | 0.852 | 0.977 | 0.946 | 0.870 | 1 |
| IFI27_IFI44L_SLC1A2_IL1R2_MMP8 | 0.887 | 0.879 | 0.895 | 0.922 | 0.876 | 0.968 | 0.929 | 0.864 | 0.995 |
| RSAD2_IFI27_HERC6_RETN_MMP8 | 0.887 | 0.878 | 0.896 | 0.911 | 0.850 | 0.972 | 0.933 | 0.868 | 0.997 |
| IFI27_OAS1_ISG15_IL1R2_RETN | 0.887 | 0.879 | 0.895 | 0.919 | 0.866 | 0.971 | 0.940 | 0.882 | 0.998 |
| IFI27_IFIT1_IFI44L_FAM20A_RETN | 0.887 | 0.877 | 0.897 | 0.910 | 0.846 | 0.974 | 0.943 | 0.866 | 1 |
| IFI27_SIGLEC1_HERC6_FAM20A_MMP8 | 0.887 | 0.877 | 0.897 | 0.904 | 0.836 | 0.973 | 0.943 | 0.865 | 1 |
| IFI27_IFIT1_HERC6_RETN_MMP8 | 0.887 | 0.877 | 0.896 | 0.908 | 0.845 | 0.970 | 0.930 | 0.863 | 0.998 |
| IFI27_IFIT1_SLC1A2_IL1R2_RETN | 0.887 | 0.878 | 0.895 | 0.919 | 0.867 | 0.971 | 0.939 | 0.878 | 1 |
| IFI27_OAS1_IFIT1_ISG15_IL1R2 | 0.887 | 0.879 | 0.895 | 0.915 | 0.864 | 0.966 | 0.943 | 0.890 | 0.997 |
| IFI27_HERC6_SLC1A2_FAM20A_RETN | 0.887 | 0.877 | 0.896 | 0.919 | 0.861 | 0.976 | 0.940 | 0.860 | 1 |
| RSAD2_IFI27_IFIT1_ISG15_IL1R2 | 0.887 | 0.879 | 0.894 | 0.915 | 0.864 | 0.966 | 0.941 | 0.888 | 0.994 |
| SIGLEC1_ISG15_IL1R2_OLAH_FAM20A | 0.887 | 0.878 | 0.895 | 0.908 | 0.845 | 0.972 | 0.963 | 0.912 | 1 |
| IFI27_IFI44L_ISG15_HERC6_RETN | 0.886 | 0.877 | 0.896 | 0.908 | 0.846 | 0.971 | 0.939 | 0.873 | 1 |
| RSAD2_SIGLEC1_IL1R2_OLAH_FAM20A | 0.886 | 0.877 | 0.895 | 0.909 | 0.846 | 0.972 | 0.963 | 0.912 | 1 |
| IFI27_OAS1_IFIT1_SLC1A2_IL1R2 | 0.886 | 0.878 | 0.895 | 0.922 | 0.876 | 0.968 | 0.937 | 0.875 | 0.999 |
| IFI27_OAS1_IFI44L_IL1R2_MMP8 | 0.886 | 0.879 | 0.894 | 0.915 | 0.864 | 0.965 | 0.930 | 0.871 | 0.989 |
| IFI27_OAS1_IFI44L_SIGLEC1_RETN | 0.886 | 0.877 | 0.895 | 0.910 | 0.849 | 0.970 | 0.954 | 0.894 | 1 |
| IFI44L_SIGLEC1_IL1R2_FAM20A_RETN | 0.886 | 0.877 | 0.895 | 0.907 | 0.844 | 0.971 | 0.962 | 0.909 | 1 |
| IFI27_IFIT1_SIGLEC1_ISG15_RETN | 0.886 | 0.877 | 0.895 | 0.908 | 0.846 | 0.970 | 0.955 | 0.900 | 1 |
| IFI27_OAS1_HERC6_SLC1A2_RETN | 0.886 | 0.877 | 0.895 | 0.915 | 0.858 | 0.973 | 0.932 | 0.857 | 1 |
| SIGLEC1_ISG15_IL1R2_FAM20A_RETN | 0.886 | 0.877 | 0.895 | 0.906 | 0.842 | 0.971 | 0.962 | 0.907 | 1 |
| IFI27_OAS1_HERC6_SLC1A2_IL1R2 | 0.886 | 0.878 | 0.894 | 0.920 | 0.875 | 0.966 | 0.918 | 0.845 | 0.992 |
| SIGLEC1_HERC6_IL1R2_FAM20A_RETN | 0.886 | 0.877 | 0.895 | 0.911 | 0.848 | 0.973 | 0.959 | 0.898 | 1 |
| IFI27_OAS1_SIGLEC1_ISG15_RETN | 0.886 | 0.877 | 0.895 | 0.910 | 0.848 | 0.971 | 0.953 | 0.892 | 1 |
| IFI27_OAS1_SIGLEC1_SLC1A2_RETN | 0.886 | 0.877 | 0.894 | 0.915 | 0.857 | 0.972 | 0.948 | 0.880 | 1 |
| RSAD2_IFI44L_SIGLEC1_IL1R2_FAM20A | 0.886 | 0.877 | 0.895 | 0.911 | 0.848 | 0.973 | 0.964 | 0.914 | 1 |
| RSAD2_IFI27_HERC6_SLC1A2_IL1R2 | 0.886 | 0.878 | 0.894 | 0.920 | 0.875 | 0.966 | 0.929 | 0.863 | 0.995 |
| IFI27_OAS1_SIGLEC1_SLC1A2_FAM20A | 0.886 | 0.876 | 0.895 | 0.918 | 0.859 | 0.977 | 0.946 | 0.872 | 1 |
| IFIT1_SIGLEC1_SLC1A2_IL1R2_FAM20A | 0.886 | 0.877 | 0.894 | 0.917 | 0.863 | 0.971 | 0.951 | 0.891 | 1 |
| RSAD2_IFI27_SLC1A2_IL1R2_RETN | 0.886 | 0.877 | 0.894 | 0.922 | 0.870 | 0.973 | 0.929 | 0.860 | 0.998 |
| IFIT1_SIGLEC1_SLC1A2_OLAH_FAM20A | 0.886 | 0.876 | 0.895 | 0.919 | 0.864 | 0.974 | 0.945 | 0.885 | 1 |
| SIGLEC1_HERC6_SLC1A2_IL1R2_FAM20A | 0.886 | 0.876 | 0.895 | 0.917 | 0.864 | 0.971 | 0.948 | 0.875 | 1 |
| IFI44L_SIGLEC1_SLC1A2_IL1R2_FAM20A | 0.886 | 0.877 | 0.894 | 0.916 | 0.862 | 0.970 | 0.953 | 0.886 | 1 |
| IFI27_OAS1_SLC1A2_IL1R2_RETN | 0.886 | 0.877 | 0.894 | 0.922 | 0.872 | 0.972 | 0.926 | 0.856 | 0.997 |
| IFI27_IFI44L_ISG15_SLC1A2_IL1R2 | 0.886 | 0.878 | 0.893 | 0.922 | 0.877 | 0.968 | 0.933 | 0.868 | 0.997 |
| IFI44L_SIGLEC1_SLC1A2_OLAH_FAM20A | 0.886 | 0.876 | 0.895 | 0.912 | 0.853 | 0.971 | 0.949 | 0.887 | 1 |
| RSAD2_IFI27_SIGLEC1_SLC1A2_FAM20A | 0.886 | 0.876 | 0.895 | 0.919 | 0.859 | 0.978 | 0.947 | 0.873 | 1 |
| IFIT1_IFI44L_SIGLEC1_ISG15_FAM20A | 0.885 | 0.877 | 0.894 | 0.899 | 0.829 | 0.969 | 0.940 | 0.878 | 1 |
| SIGLEC1_SLC1A2_IL1R2_OLAH_FAM20A | 0.885 | 0.877 | 0.894 | 0.915 | 0.860 | 0.970 | 0.954 | 0.893 | 1 |
| IFI27_IFI44L_HERC6_RETN_MMP8 | 0.885 | 0.876 | 0.895 | 0.907 | 0.844 | 0.970 | 0.932 | 0.863 | 1 |
| IFI27_IFIT1_SLC1A2_IL1R2_MMP8 | 0.885 | 0.877 | 0.893 | 0.922 | 0.876 | 0.968 | 0.936 | 0.875 | 0.997 |
| RSAD2_SIGLEC1_IL1R2_FAM20A_RETN | 0.885 | 0.876 | 0.895 | 0.908 | 0.845 | 0.972 | 0.962 | 0.907 | 1 |
| SIGLEC1_IL1R2_OLAH_FAM20A_RETN | 0.885 | 0.876 | 0.894 | 0.907 | 0.843 | 0.971 | 0.962 | 0.907 | 1 |
| RSAD2_SIGLEC1_SLC1A2_IL1R2_FAM20A | 0.885 | 0.876 | 0.894 | 0.916 | 0.862 | 0.970 | 0.954 | 0.890 | 1 |
| RSAD2_IFIT1_SIGLEC1_OLAH_FAM20A | 0.885 | 0.876 | 0.894 | 0.904 | 0.834 | 0.973 | 0.953 | 0.902 | 1 |
| OAS1_SIGLEC1_IL1R2_FAM20A_RETN | 0.885 | 0.876 | 0.894 | 0.907 | 0.844 | 0.971 | 0.962 | 0.907 | 1 |
| IFI27_IFIT1_IFI44L_SIGLEC1_RETN | 0.885 | 0.876 | 0.894 | 0.908 | 0.846 | 0.970 | 0.951 | 0.894 | 1 |
| OAS1_SIGLEC1_ISG15_IL1R2_FAM20A | 0.885 | 0.876 | 0.894 | 0.909 | 0.846 | 0.973 | 0.964 | 0.914 | 1 |
| RSAD2_IFI27_IFIT1_SIGLEC1_RETN | 0.885 | 0.876 | 0.894 | 0.908 | 0.846 | 0.970 | 0.955 | 0.900 | 1 |
| RSAD2_IFI27_IFIT1_SLC1A2_IL1R2 | 0.885 | 0.877 | 0.893 | 0.922 | 0.876 | 0.968 | 0.936 | 0.875 | 0.997 |
| IFI27_IFI44L_SIGLEC1_ISG15_RETN | 0.885 | 0.876 | 0.894 | 0.906 | 0.844 | 0.969 | 0.952 | 0.895 | 1 |
| RSAD2_IFI27_ISG15_IL1R2_MMP8 | 0.885 | 0.877 | 0.893 | 0.915 | 0.864 | 0.966 | 0.936 | 0.879 | 0.992 |
| IFI27_SIGLEC1_ISG15_HERC6_RETN | 0.885 | 0.875 | 0.895 | 0.906 | 0.841 | 0.971 | 0.963 | 0.916 | 1 |
| IFI27_IFIT1_ISG15_FAM20A_RETN | 0.885 | 0.875 | 0.895 | 0.911 | 0.849 | 0.972 | 0.942 | 0.865 | 1 |
| RSAD2_IFI27_IFI44L_ISG15_IL1R2 | 0.885 | 0.877 | 0.892 | 0.915 | 0.864 | 0.966 | 0.936 | 0.880 | 0.992 |
| RSAD2_SIGLEC1_ISG15_IL1R2_FAM20A | 0.885 | 0.876 | 0.894 | 0.909 | 0.845 | 0.972 | 0.963 | 0.912 | 1 |
| IFI27_OAS1_SIGLEC1_RETN_MMP8 | 0.885 | 0.875 | 0.894 | 0.911 | 0.850 | 0.972 | 0.957 | 0.897 | 1 |
| SIGLEC1_SLC1A2_IL1R2_FAM20A_RETN | 0.885 | 0.876 | 0.894 | 0.916 | 0.862 | 0.969 | 0.953 | 0.885 | 1 |
| RSAD2_IFI27_OAS1_SLC1A2_IL1R2 | 0.885 | 0.877 | 0.893 | 0.921 | 0.875 | 0.967 | 0.930 | 0.862 | 0.999 |
| IFI27_ISG15_SLC1A2_IL1R2_RETN | 0.885 | 0.877 | 0.893 | 0.919 | 0.868 | 0.970 | 0.918 | 0.839 | 0.998 |
| IFI27_IFIT1_HERC6_SLC1A2_IL1R2 | 0.885 | 0.876 | 0.893 | 0.922 | 0.876 | 0.968 | 0.938 | 0.878 | 0.998 |
| OAS1_IFIT1_IFI44L_SIGLEC1_FAM20A | 0.885 | 0.875 | 0.894 | 0.902 | 0.832 | 0.972 | 0.938 | 0.870 | 1 |
| SIGLEC1_ISG15_SLC1A2_IL1R2_FAM20A | 0.885 | 0.876 | 0.893 | 0.914 | 0.859 | 0.969 | 0.953 | 0.888 | 1 |
| IFI27_SLC1A2_IL1R2_RETN_MMP8 | 0.884 | 0.876 | 0.893 | 0.921 | 0.872 | 0.970 | 0.915 | 0.832 | 0.998 |
| RSAD2_IFI27_OAS1_ISG15_IL1R2 | 0.884 | 0.877 | 0.892 | 0.915 | 0.865 | 0.966 | 0.939 | 0.884 | 0.994 |
| RSAD2_IFI27_IFI44L_FAM20A_RETN | 0.884 | 0.874 | 0.894 | 0.910 | 0.846 | 0.974 | 0.942 | 0.865 | 1 |
| IFI27_IFIT1_ISG15_SLC1A2_IL1R2 | 0.884 | 0.876 | 0.893 | 0.922 | 0.876 | 0.968 | 0.937 | 0.877 | 0.997 |
| RSAD2_IFI27_SLC1A2_IL1R2_MMP8 | 0.884 | 0.877 | 0.892 | 0.921 | 0.874 | 0.969 | 0.934 | 0.870 | 0.997 |
| IFI27_OAS1_IFI44L_ISG15_IL1R2 | 0.884 | 0.877 | 0.892 | 0.914 | 0.863 | 0.964 | 0.932 | 0.873 | 0.990 |
| IFI27_IFI44L_HERC6_SLC1A2_IL1R2 | 0.884 | 0.876 | 0.893 | 0.921 | 0.875 | 0.966 | 0.929 | 0.864 | 0.995 |
| IFI27_IFIT1_HERC6_FAM20A_MMP8 | 0.884 | 0.875 | 0.894 | 0.911 | 0.851 | 0.970 | 0.926 | 0.842 | 1 |
| IFI27_IFIT1_IFI44L_SLC1A2_IL1R2 | 0.884 | 0.876 | 0.892 | 0.922 | 0.875 | 0.968 | 0.939 | 0.880 | 0.998 |
| IFI27_OAS1_ISG15_IL1R2_MMP8 | 0.884 | 0.876 | 0.892 | 0.914 | 0.864 | 0.965 | 0.932 | 0.873 | 0.990 |
| OAS1_IFIT1_SIGLEC1_FAM20A_RETN | 0.884 | 0.875 | 0.893 | 0.903 | 0.834 | 0.972 | 0.949 | 0.889 | 1 |
| OAS1_SIGLEC1_SLC1A2_IL1R2_FAM20A | 0.884 | 0.875 | 0.893 | 0.916 | 0.861 | 0.970 | 0.953 | 0.890 | 1 |
| RSAD2_IFI27_SIGLEC1_ISG15_RETN | 0.884 | 0.875 | 0.893 | 0.909 | 0.847 | 0.970 | 0.953 | 0.896 | 1 |
| OAS1_SIGLEC1_SLC1A2_OLAH_FAM20A | 0.884 | 0.874 | 0.893 | 0.915 | 0.857 | 0.972 | 0.951 | 0.895 | 1 |
| IFI44L_SIGLEC1_OLAH_FAM20A_MMP8 | 0.884 | 0.874 | 0.893 | 0.902 | 0.834 | 0.970 | 0.966 | 0.923 | 1 |
| SIGLEC1_ISG15_SLC1A2_OLAH_FAM20A | 0.884 | 0.874 | 0.893 | 0.913 | 0.854 | 0.971 | 0.950 | 0.891 | 1 |
| SIGLEC1_HERC6_SLC1A2_OLAH_FAM20A | 0.883 | 0.874 | 0.893 | 0.914 | 0.857 | 0.972 | 0.947 | 0.874 | 1 |
| IFIT1_IFI44L_SIGLEC1_FAM20A_MMP8 | 0.883 | 0.874 | 0.893 | 0.901 | 0.832 | 0.970 | 0.938 | 0.869 | 1 |
| IFI27_OAS1_IFI44L_FAM20A_RETN | 0.883 | 0.874 | 0.893 | 0.911 | 0.848 | 0.974 | 0.946 | 0.869 | 1 |
| IFI27_IFIT1_SIGLEC1_RETN_MMP8 | 0.883 | 0.874 | 0.893 | 0.909 | 0.847 | 0.971 | 0.957 | 0.902 | 1 |
| RSAD2_SIGLEC1_SLC1A2_OLAH_FAM20A | 0.883 | 0.874 | 0.893 | 0.915 | 0.858 | 0.972 | 0.950 | 0.891 | 1 |
| RSAD2_IFI27_IFI44L_HERC6_FAM20A | 0.883 | 0.873 | 0.893 | 0.907 | 0.843 | 0.970 | 0.930 | 0.851 | 1 |
| RSAD2_IFI27_IFI44L_SIGLEC1_RETN | 0.883 | 0.874 | 0.892 | 0.908 | 0.846 | 0.969 | 0.951 | 0.893 | 1 |
| IFI27_SIGLEC1_ISG15_SLC1A2_FAM20A | 0.883 | 0.874 | 0.893 | 0.911 | 0.850 | 0.971 | 0.948 | 0.872 | 1 |
| OAS1_IFIT1_SIGLEC1_{——}ISG15_FAM20A | 0.883 | 0.874 | 0.893 | 0.900 | 0.829 | 0.970 | 0.946 | 0.879 | 1 |
| IFI44L_SLC1A2_OLAH_FAM20A_MMP8 | 0.883 | 0.874 | 0.892 | 0.911 | 0.854 | 0.969 | 0.929 | 0.855 | 1 |
| RSAD2_IFI27_ISG15_SLC1A2_IL1R2 | 0.883 | 0.875 | 0.891 | 0.922 | 0.876 | 0.968 | 0.929 | 0.863 | 0.996 |
| IFI27_ISG15_HERC6_SLC1A2_IL1R2 | 0.883 | 0.875 | 0.891 | 0.922 | 0.877 | 0.967 | 0.925 | 0.858 | 0.992 |
| IFI27_IFIT1_FAM20A_RETN_MMP8 | 0.883 | 0.873 | 0.893 | 0.912 | 0.850 | 0.974 | 0.943 | 0.866 | 1 |
| IFI44L_ISG15_HERC6_IL1R2_FAM20A | 0.883 | 0.874 | 0.892 | 0.907 | 0.846 | 0.967 | 0.940 | 0.865 | 1 |
| IFIT1_IFI44L_SIGLEC1_FAM20A_RETN | 0.883 | 0.874 | 0.892 | 0.900 | 0.831 | 0.970 | 0.941 | 0.880 | 1 |
| IFI27_IFI44L_SIGLEC1_SLC1A2_FAM20A | 0.883 | 0.873 | 0.892 | 0.911 | 0.851 | 0.972 | 0.947 | 0.870 | 1 |
| RSAD2_OAS1_IFIT1_SIGLEC1_FAM20A | 0.883 | 0.873 | 0.892 | 0.899 | 0.828 | 0.970 | 0.947 | 0.880 | 1 |
| IFI27_OAS1_ISG15_FAM20A_RETN | 0.883 | 0.873 | 0.892 | 0.911 | 0.849 | 0.972 | 0.943 | 0.866 | 1 |
| IFIT1_IFI44L_SIGLEC1_SLC1A2_FAM20A | 0.883 | 0.874 | 0.892 | 0.911 | 0.852 | 0.970 | 0.936 | 0.861 | 1 |
| RSAD2_IFI27_HERC6_FAM20A_MMP8 | 0.883 | 0.873 | 0.892 | 0.911 | 0.852 | 0.969 | 0.927 | 0.846 | 1 |
| RSAD2_IFI27_IFI44L_SLC1A2_IL1R2 | 0.883 | 0.875 | 0.890 | 0.921 | 0.875 | 0.967 | 0.933 | 0.868 | 0.997 |
| SIGLEC1_HERC6_OLAH_FAM20A_MMP8 | 0.883 | 0.872 | 0.893 | 0.902 | 0.835 | 0.970 | 0.963 | 0.914 | 1 |
| SIGLEC1_OLAH_FAM20A_RETN_MMP8 | 0.882 | 0.872 | 0.893 | 0.905 | 0.838 | 0.973 | 0.970 | 0.929 | 1 |
| IFIT1_SIGLEC1_ISG15_OLAH_FAM20A | 0.882 | 0.873 | 0.892 | 0.901 | 0.832 | 0.970 | 0.948 | 0.893 | 1 |
| SIGLEC1_ISG15_OLAH_FAM20A_MMP8 | 0.882 | 0.873 | 0.892 | 0.902 | 0.834 | 0.970 | 0.963 | 0.919 | 1 |
| RSAD2_SIGLEC1_HERC6_OLAH_FAM20A | 0.882 | 0.873 | 0.892 | 0.907 | 0.844 | 0.969 | 0.946 | 0.888 | 1 |
| IFI27_ISG15_SLC1A2_IL1R2_MMP8 | 0.882 | 0.875 | 0.890 | 0.919 | 0.872 | 0.966 | 0.921 | 0.847 | 0.994 |
| RSAD2_IFI27_SIGLEC1_RETN_MMP8 | 0.882 | 0.873 | 0.892 | 0.912 | 0.852 | 0.973 | 0.957 | 0.902 | 1 |
| ISG15_HERC6_IL1R2_OLAH_FAM20A | 0.882 | 0.873 | 0.892 | 0.906 | 0.846 | 0.966 | 0.935 | 0.858 | 1 |
| IFIT1_ISG15_HERC6_IL1R2_FAM20A | 0.882 | 0.873 | 0.891 | 0.905 | 0.844 | 0.965 | 0.933 | 0.851 | 1 |
| SIGLEC1_SLC1A2_OLAH_FAM20A_RETN | 0.882 | 0.873 | 0.892 | 0.913 | 0.854 | 0.972 | 0.950 | 0.890 | 1 |
| IFI27_OAS1_HERC6_FAM20A_MMP8 | 0.882 | 0.872 | 0.892 | 0.910 | 0.851 | 0.969 | 0.927 | 0.846 | 1 |
| IFI44L_SIGLEC1_HERC6_OLAH_FAM20A | 0.882 | 0.872 | 0.892 | 0.903 | 0.836 | 0.969 | 0.955 | 0.896 | 1 |
| IFI44L_SIGLEC1_SLC1A2_OLAH_MMP8 | 0.882 | 0.874 | 0.891 | 0.912 | 0.856 | 0.967 | 0.957 | 0.914 | 0.999 |
| IFIT1_SIGLEC1_FAM20A_RETN_MMP8 | 0.882 | 0.873 | 0.891 | 0.906 | 0.840 | 0.972 | 0.947 | 0.885 | 1 |
| IFI27_HERC6_SLC1A2_IL1R2_MMP8 | 0.882 | 0.874 | 0.890 | 0.920 | 0.874 | 0.967 | 0.925 | 0.857 | 0.993 |
| OAS1_IFIT1_SIGLEC1_FAM20A_MMP8 | 0.882 | 0.873 | 0.892 | 0.901 | 0.832 | 0.971 | 0.948 | 0.881 | 1 |
| RSAD2_IFI27_OAS1_FAM20A_RETN | 0.882 | 0.873 | 0.892 | 0.909 | 0.847 | 0.970 | 0.937 | 0.858 | 1 |
| RSAD2_IFI27_HERC6_SLC1A2_RETN | 0.882 | 0.873 | 0.891 | 0.912 | 0.853 | 0.971 | 0.934 | 0.864 | 1 |
| IFI27_IFIT1_SIGLEC1_SLC1A2_RETN | 0.882 | 0.873 | 0.891 | 0.915 | 0.859 | 0.971 | 0.955 | 0.900 | 1 |
| IFI27_IFI44L_SIGLEC1_RETN_MMP8 | 0.882 | 0.873 | 0.891 | 0.907 | 0.844 | 0.969 | 0.953 | 0.897 | 1 |
| OAS1_ISG15_HERC6_IL1R2_FAM20A | 0.882 | 0.873 | 0.891 | 0.904 | 0.843 | 0.965 | 0.933 | 0.850 | 1 |
| IFI27_OAS1_IFIT1_FAM20A_RETN | 0.882 | 0.872 | 0.892 | 0.909 | 0.846 | 0.971 | 0.942 | 0.864 | 1 |
| IFI27_ISG15_HERC6_RETN_MMP8 | 0.882 | 0.872 | 0.892 | 0.905 | 0.840 | 0.969 | 0.947 | 0.886 | 1 |
| RSAD2_ISG15_HERC6_IL1R2_FAM20A | 0.882 | 0.872 | 0.891 | 0.906 | 0.846 | 0.966 | 0.932 | 0.848 | 1 |
| ISG15_HERC6_IL1R2_FAM20A_MMP8 | 0.882 | 0.873 | 0.891 | 0.907 | 0.848 | 0.965 | 0.927 | 0.841 | 1 |
| IFI27_OAS1_SLC1A2_IL1R2_MMP8 | 0.882 | 0.874 | 0.890 | 0.920 | 0.874 | 0.967 | 0.924 | 0.856 | 0.992 |
| IFI27_OAS1_IFI44L_SLC1A2_IL1R2 | 0.882 | 0.873 | 0.890 | 0.922 | 0.877 | 0.967 | 0.929 | 0.865 | 0.994 |
| IFI27_IFI44L_SLC1A2_FAM20A_RETN | 0.882 | 0.872 | 0.891 | 0.911 | 0.850 | 0.973 | 0.941 | 0.861 | 1 |
| IFI44L_SLC1A2_IL1R2_OLAH_FAM20A | 0.882 | 0.873 | 0.891 | 0.912 | 0.855 | 0.968 | 0.928 | 0.852 | 1 |
| RSAD2_IFIT1_IFI44L_SIGLEC1_FAM20A | 0.882 | 0.873 | 0.891 | 0.899 | 0.829 | 0.970 | 0.939 | 0.875 | 1 |
| IFI27_SIGLEC1_HERC6_RETN_MMP8 | 0.882 | 0.872 | 0.891 | 0.904 | 0.838 | 0.969 | 0.961 | 0.912 | 1 |
| IFI44L_HERC6_IL1R2_OLAH_FAM20A | 0.882 | 0.872 | 0.891 | 0.905 | 0.844 | 0.967 | 0.938 | 0.864 | 1 |
| ISG15_HERC6_IL1R2_FAM20A_RETN | 0.882 | 0.872 | 0.891 | 0.907 | 0.847 | 0.967 | 0.933 | 0.849 | 1 |
| HERC6_IL1R2_OLAH_FAM20A_MMP8 | 0.881 | 0.872 | 0.890 | 0.908 | 0.851 | 0.965 | 0.932 | 0.851 | 1 |
| RSAD2_SIGLEC1_OLAH_FAM20A_MMP8 | 0.881 | 0.871 | 0.891 | 0.904 | 0.838 | 0.971 | 0.964 | 0.924 | 1 |
| IFI27_SIGLEC1_SLC1A2_FAM20A_MMP8 | 0.881 | 0.872 | 0.891 | 0.909 | 0.846 | 0.972 | 0.947 | 0.870 | 1 |
| RSAD2_IFI27_SIGLEC1_SLC1A2_RETN | 0.881 | 0.872 | 0.890 | 0.914 | 0.857 | 0.971 | 0.951 | 0.893 | 1 |
| RSAD2_IFI27_IFIT1_FAM20A_RETN | 0.881 | 0.871 | 0.891 | 0.911 | 0.849 | 0.972 | 0.942 | 0.865 | 1 |
| IFIT1_SIGLEC1_OLAH_FAM20A_RETN | 0.881 | 0.872 | 0.890 | 0.903 | 0.833 | 0.972 | 0.948 | 0.893 | 1 |
| IFIT1_SIGLEC1_SLC1A2_OLAH_MMP8 | 0.881 | 0.872 | 0.890 | 0.911 | 0.856 | 0.967 | 0.954 | 0.911 | 0.998 |
| IFI27_OAS1_ISG15_SLC1A2_IL1R2 | 0.881 | 0.873 | 0.889 | 0.922 | 0.876 | 0.967 | 0.923 | 0.853 | 0.992 |
| RSAD2_IFI27_ISG15_FAM20A_RETN | 0.881 | 0.871 | 0.891 | 0.910 | 0.848 | 0.971 | 0.942 | 0.864 | 1 |
| SIGLEC1_ISG15_HERC6_OLAH_FAM20A | 0.881 | 0.871 | 0.891 | 0.902 | 0.835 | 0.970 | 0.957 | 0.901 | 1 |
| IFI27_IFI44L_HERC6_FAM20A_MMP8 | 0.881 | 0.871 | 0.891 | 0.906 | 0.844 | 0.969 | 0.932 | 0.851 | 1 |
| IFI44L_SIGLEC1_HERC6_IL1R2_MMP8 | 0.881 | 0.873 | 0.889 | 0.902 | 0.843 | 0.961 | 0.961 | 0.921 | 1 |
| IFI27_ISG15_HERC6_FAM20A_MMP8 | 0.881 | 0.871 | 0.891 | 0.907 | 0.844 | 0.969 | 0.932 | 0.851 | 1 |
| OAS1_IFIT1_SIGLEC1_HERC6_IL1R2 | 0.881 | 0.872 | 0.889 | 0.903 | 0.844 | 0.962 | 0.952 | 0.907 | 0.997 |
| OAS1_IFIT1_SIGLEC1_SLC1A2_FAM20A | 0.881 | 0.871 | 0.890 | 0.907 | 0.846 | 0.968 | 0.945 | 0.873 | 1 |
| IFI27_IFI44L_SIGLEC1_SLC1A2_RETN | 0.881 | 0.872 | 0.889 | 0.915 | 0.858 | 0.972 | 0.953 | 0.896 | 1 |
| IFI27_ISG15_FAM20A_RETN_MMP8 | 0.881 | 0.870 | 0.891 | 0.909 | 0.848 | 0.970 | 0.933 | 0.850 | 1 |
| IFI44L_HERC6_IL1R2_FAM20A_MMP8 | 0.881 | 0.872 | 0.890 | 0.908 | 0.848 | 0.968 | 0.934 | 0.851 | 1 |
| RSAD2_OAS1_HERC6_IL1R2_FAM20A | 0.881 | 0.871 | 0.890 | 0.904 | 0.842 | 0.967 | 0.929 | 0.845 | 1 |
| IFI44L_SIGLEC1_SLC1A2_IL1R2_OLAH | 0.881 | 0.872 | 0.889 | 0.913 | 0.858 | 0.968 | 0.963 | 0.925 | 1 |
| IFI27_SIGLEC1_HERC6_SLC1A2_FAM20A | 0.881 | 0.871 | 0.890 | 0.910 | 0.847 | 0.972 | 0.949 | 0.873 | 1 |
| SIGLEC1_HERC6_FAM20A_RETN_MMP8 | 0.881 | 0.870 | 0.891 | 0.905 | 0.836 | 0.973 | 0.957 | 0.891 | 1 |
| IFI27_IFIT1_SIGLEC1_HERC6_MMP8 | 0.881 | 0.872 | 0.889 | 0.905 | 0.847 | 0.963 | 0.909 | 0.834 | 0.984 |
| IFIT1_IFI44L_HERC6_IL1R2_FAM20A | 0.881 | 0.872 | 0.889 | 0.906 | 0.845 | 0.967 | 0.935 | 0.855 | 1 |
| OAS1_HERC6_IL1R2_FAM20A_MMP8 | 0.880 | 0.872 | 0.889 | 0.905 | 0.843 | 0.966 | 0.934 | 0.853 | 1 |
| OAS1_SIGLEC1_SLC1A2_OLAH_MMP8 | 0.880 | 0.871 | 0.890 | 0.911 | 0.855 | 0.967 | 0.958 | 0.916 | 0.999 |
| IFIT1_SIGLEC1_HERC6_SLC1A2_OLAH | 0.880 | 0.871 | 0.890 | 0.912 | 0.857 | 0.967 | 0.943 | 0.893 | 0.994 |
| OAS1_SIGLEC1_HERC6_OLAH_FAM20A | 0.880 | 0.871 | 0.890 | 0.905 | 0.841 | 0.969 | 0.954 | 0.901 | 1 |
| RSAD2_SIGLEC1_SLC1A2_OLAH_MMP8 | 0.880 | 0.871 | 0.889 | 0.912 | 0.857 | 0.968 | 0.957 | 0.915 | 0.998 |
| RSAD2_IFIT1_SIGLEC1_ISG15_FAM20A | 0.880 | 0.871 | 0.889 | 0.900 | 0.830 | 0.970 | 0.945 | 0.881 | 1 |
| SIGLEC1_ISG15_SLC1A2_OLAH_MMP8 | 0.880 | 0.872 | 0.889 | 0.912 | 0.857 | 0.968 | 0.952 | 0.907 | 0.997 |
| SIGLEC1_SLC1A2_IL1R2_OLAH_MMP8 | 0.880 | 0.872 | 0.889 | 0.912 | 0.858 | 0.967 | 0.966 | 0.931 | 1 |
| OAS1_SIGLEC1_OLAH_FAM20A_MMP8 | 0.880 | 0.870 | 0.890 | 0.901 | 0.833 | 0.969 | 0.963 | 0.920 | 1 |
| IFI44L_SIGLEC1_ISG15_SLC1A2_OLAH | 0.880 | 0.872 | 0.889 | 0.911 | 0.855 | 0.967 | 0.949 | 0.901 | 0.997 |
| IFI27_IFIT1_HERC6_SLC1A2_RETN | 0.880 | 0.870 | 0.890 | 0.910 | 0.849 | 0.971 | 0.938 | 0.869 | 1 |
| IFIT1_HERC6_IL1R2_FAM20A_MMP8 | 0.880 | 0.871 | 0.889 | 0.904 | 0.843 | 0.965 | 0.937 | 0.859 | 1 |
| RSAD2_IFIT1_SIGLEC1_FAM20A_RETN | 0.880 | 0.871 | 0.890 | 0.902 | 0.833 | 0.971 | 0.947 | 0.886 | 1 |
| RSAD2_SIGLEC1_HERC6_IL1R2_MMP8 | 0.880 | 0.871 | 0.889 | 0.904 | 0.845 | 0.962 | 0.961 | 0.921 | 1 |
| OAS1_IFIT1_SIGLEC1_SLC1A2_OLAH | 0.880 | 0.871 | 0.889 | 0.911 | 0.855 | 0.967 | 0.946 | 0.896 | 0.995 |
| IFIT1_IFI44L_SIGLEC1_SLC1A2_OLAH | 0.880 | 0.871 | 0.889 | 0.913 | 0.857 | 0.969 | 0.943 | 0.892 | 0.995 |
| RSAD2_OAS1_SIGLEC1_OLAH_FAM20A | 0.880 | 0.870 | 0.890 | 0.899 | 0.828 | 0.970 | 0.959 | 0.911 | 1 |
| RSAD2_IFIT1_SIGLEC1_SLC1A2_FAM20A | 0.880 | 0.871 | 0.889 | 0.907 | 0.846 | 0.968 | 0.945 | 0.873 | 1 |
| IFIT1_SIGLEC1_ISG15_FAM20A_RETN | 0.880 | 0.871 | 0.889 | 0.899 | 0.829 | 0.968 | 0.942 | 0.880 | 1 |
| IFI27_OAS1_FAM20A_RETN_MMP8 | 0.880 | 0.870 | 0.890 | 0.908 | 0.846 | 0.971 | 0.943 | 0.866 | 1 |
| IFI44L_SLC1A2_IL1R2_FAM20A_MMP8 | 0.880 | 0.872 | 0.888 | 0.910 | 0.858 | 0.962 | 0.928 | 0.840 | 1 |
| IFI44L_ISG15_SLC1A2_OLAH_FAM20A | 0.880 | 0.870 | 0.889 | 0.909 | 0.849 | 0.968 | 0.927 | 0.853 | 1 |
| OAS1_SIGLEC1_HERC6_IL1R2_MMP8 | 0.880 | 0.871 | 0.888 | 0.903 | 0.845 | 0.962 | 0.960 | 0.919 | 1 |
| OAS1_IFIT1_HERC6_IL1R2_FAM20A | 0.880 | 0.871 | 0.889 | 0.904 | 0.842 | 0.967 | 0.936 | 0.855 | 1 |
| OAS1_SIGLEC1_HERC6_SLC1A2_OLAH | 0.880 | 0.870 | 0.889 | 0.912 | 0.857 | 0.967 | 0.951 | 0.904 | 0.998 |
| IFI27_OAS1_SIGLEC1_HERC6_MMP8 | 0.880 | 0.871 | 0.888 | 0.906 | 0.850 | 0.962 | 0.917 | 0.845 | 0.990 |
| IFIT1_SIGLEC1_HERC6_IL1R2_MMP8 | 0.880 | 0.871 | 0.888 | 0.903 | 0.845 | 0.962 | 0.957 | 0.914 | 0.999 |
| RSAD2_OAS1_SIGLEC1_HERC6_IL1R2 | 0.880 | 0.871 | 0.889 | 0.904 | 0.846 | 0.962 | 0.957 | 0.914 | 0.999 |
| OAS1_HERC6_IL1R2_OLAH_FAM20A | 0.880 | 0.870 | 0.889 | 0.906 | 0.845 | 0.968 | 0.938 | 0.864 | 1 |
| RSAD2_HERC6_IL1R2_OLAH_FAM20A | 0.880 | 0.870 | 0.889 | 0.906 | 0.845 | 0.967 | 0.934 | 0.857 | 1 |
| IFI44L_SIGLEC1_ISG15_OLAH_FAM20A | 0.880 | 0.870 | 0.889 | 0.897 | 0.825 | 0.968 | 0.961 | 0.916 | 1 |
| IFIT1_SIGLEC1_ISG15_FAM20A_MMP8 | 0.880 | 0.870 | 0.889 | 0.899 | 0.830 | 0.969 | 0.940 | 0.872 | 1 |
| IFI27_SIGLEC1_ISG15_SLC1A2_RETN | 0.880 | 0.870 | 0.889 | 0.910 | 0.849 | 0.970 | 0.957 | 0.904 | 1 |
| IFI27_IFIT1_IFI44L_HERC6_FAM20A | 0.879 | 0.870 | 0.889 | 0.899 | 0.833 | 0.964 | 0.927 | 0.845 | 1 |
| RSAD2_SIGLEC1_SLC1A2_IL1R2_OLAH | 0.879 | 0.870 | 0.888 | 0.912 | 0.856 | 0.967 | 0.961 | 0.921 | 1 |
| IFIT1_SIGLEC1_ISG15_SLC1A2_OLAH | 0.879 | 0.871 | 0.888 | 0.912 | 0.857 | 0.968 | 0.945 | 0.893 | 0.996 |
| IFIT1_HERC6_IL1R2_OLAH_FAM20A | 0.879 | 0.870 | 0.889 | 0.905 | 0.845 | 0.966 | 0.936 | 0.861 | 1 |
| RSAD2_SIGLEC1_HERC6_FAM20A_RETN | 0.879 | 0.870 | 0.888 | 0.907 | 0.844 | 0.970 | 0.943 | 0.876 | 1 |
| OAS1_IFI44L_HERC6_IL1R2_FAM20A | 0.879 | 0.870 | 0.888 | 0.905 | 0.843 | 0.967 | 0.937 | 0.858 | 1 |
| IFIT1_IFI44L_SIGLEC1_HERC6_IL1R2 | 0.879 | 0.871 | 0.888 | 0.903 | 0.843 | 0.962 | 0.954 | 0.911 | 0.998 |
| IFI44L_ISG15_SLC1A2_IL1R2_FAM20A | 0.879 | 0.870 | 0.888 | 0.907 | 0.852 | 0.963 | 0.926 | 0.839 | 1 |
| IFI27_SIGLEC1_HERC6_SLC1A2_RETN | 0.879 | 0.869 | 0.889 | 0.909 | 0.847 | 0.971 | 0.953 | 0.895 | 1 |
| IFIT1_SIGLEC1_ISG15_SLC1A2_FAM20A | 0.879 | 0.870 | 0.888 | 0.907 | 0.846 | 0.967 | 0.938 | 0.865 | 1 |
| RSAD2_IFI27_FAM20A_RETN_MMP8 | 0.879 | 0.869 | 0.889 | 0.910 | 0.847 | 0.973 | 0.943 | 0.865 | 1 |
| OAS1_IFI44L_SIGLEC1_HERC6_IL1R2 | 0.879 | 0.870 | 0.888 | 0.903 | 0.843 | 0.963 | 0.955 | 0.912 | 0.999 |
| IFIT1_SIGLEC1_SLC1A2_FAM20A_MMP8 | 0.879 | 0.870 | 0.888 | 0.905 | 0.844 | 0.967 | 0.940 | 0.867 | 1 |
| IFIT1_SIGLEC1_HERC6_IL1R2_RETN | 0.879 | 0.870 | 0.888 | 0.904 | 0.841 | 0.967 | 0.952 | 0.907 | 0.997 |
| SIGLEC1_ISG15_SLC1A2_IL1R2_OLAH | 0.879 | 0.870 | 0.887 | 0.913 | 0.858 | 0.968 | 0.962 | 0.924 | 1 |
| IFIT1_SIGLEC1_SLC1A2_IL1R2_OLAH | 0.879 | 0.870 | 0.888 | 0.914 | 0.859 | 0.969 | 0.965 | 0.929 | 1 |
| RSAD2_IFIT1_SIGLEC1_FAM20A_MMP8 | 0.879 | 0.869 | 0.888 | 0.900 | 0.830 | 0.970 | 0.943 | 0.877 | 1 |
| RSAD2_SIGLEC1_ISG15_HERC6_FAM20A | 0.879 | 0.870 | 0.888 | 0.902 | 0.838 | 0.967 | 0.943 | 0.875 | 1 |
| IFI44L_SIGLEC1_HERC6_FAM20A_RETN | 0.879 | 0.869 | 0.889 | 0.903 | 0.836 | 0.970 | 0.954 | 0.888 | 1 |
| RSAD2_SIGLEC1_HERC6_SLC1A2_OLAH | 0.879 | 0.869 | 0.888 | 0.912 | 0.856 | 0.967 | 0.947 | 0.898 | 0.995 |
| RSAD2_IFIT1_SIGLEC1_SLC1A2_OLAH | 0.879 | 0.870 | 0.888 | 0.913 | 0.857 | 0.969 | 0.948 | 0.899 | 0.997 |
| IFI44L_HERC6_IL1R2_FAM20A_RETN | 0.879 | 0.870 | 0.888 | 0.907 | 0.845 | 0.968 | 0.937 | 0.857 | 1 |
| IFI44L_SIGLEC1_SLC1A2_OLAH_RETN | 0.879 | 0.870 | 0.888 | 0.914 | 0.857 | 0.970 | 0.950 | 0.901 | 0.999 |
| RSAD2_HERC6_IL1R2_FAM20A_MMP8 | 0.879 | 0.870 | 0.888 | 0.904 | 0.844 | 0.965 | 0.928 | 0.842 | 1 |
| IFI44L_SIGLEC1_ISG15_HERC6_IL1R2 | 0.879 | 0.870 | 0.887 | 0.902 | 0.842 | 0.962 | 0.955 | 0.912 | 0.999 |
| SIGLEC1_HERC6_OLAH_FAM20A_RETN | 0.879 | 0.869 | 0.889 | 0.902 | 0.834 | 0.970 | 0.960 | 0.905 | 1 |
| RSAD2_OAS1_SIGLEC1_SLC1A2_OLAH | 0.879 | 0.869 | 0.888 | 0.911 | 0.855 | 0.967 | 0.950 | 0.901 | 0.999 |
| OAS1_IL1R2_OLAH_FAM20A_MMP8 | 0.879 | 0.870 | 0.888 | 0.905 | 0.843 | 0.967 | 0.937 | 0.859 | 1 |
| IFIT1_SIGLEC1_ISG15_HERC6_IL1R2 | 0.879 | 0.870 | 0.887 | 0.905 | 0.846 | 0.963 | 0.952 | 0.907 | 0.998 |
| RSAD2_IFI27_ISG15_HERC6_FAM20A | 0.879 | 0.869 | 0.888 | 0.908 | 0.848 | 0.968 | 0.925 | 0.841 | 1 |
| IFI27_SIGLEC1_ISG15_RETN_MMP8 | 0.879 | 0.869 | 0.889 | 0.904 | 0.839 | 0.969 | 0.958 | 0.905 | 1 |
| OAS1_SIGLEC1_HERC6_FAM20A_RETN | 0.878 | 0.869 | 0.888 | 0.902 | 0.838 | 0.967 | 0.948 | 0.882 | 1 |
| SIGLEC1_HERC6_SLC1A2_OLAH_MMP8 | 0.878 | 0.869 | 0.888 | 0.911 | 0.855 | 0.967 | 0.955 | 0.913 | 0.998 |
| OAS1_IFIT1_IL1R2_FAM20A_MMP8 | 0.878 | 0.870 | 0.887 | 0.905 | 0.843 | 0.968 | 0.943 | 0.870 | 1 |
| IFI27_OAS1_ISG15_HERC6_FAM20A | 0.878 | 0.868 | 0.889 | 0.905 | 0.844 | 0.966 | 0.926 | 0.843 | 1 |
| IFI27_IFI44L_HERC6_SLC1A2_RETN | 0.878 | 0.869 | 0.888 | 0.910 | 0.849 | 0.970 | 0.938 | 0.868 | 1 |
| RSAD2_IFIT1_SIGLEC1_HERC6_IL1R2 | 0.878 | 0.870 | 0.887 | 0.903 | 0.844 | 0.962 | 0.952 | 0.907 | 0.997 |
| IFI44L_SIGLEC1_HERC6_IL1R2_OLAH | 0.878 | 0.870 | 0.887 | 0.904 | 0.843 | 0.964 | 0.959 | 0.917 | 1 |
| HERC6_IL1R2_OLAH_FAM20A_RETN | 0.878 | 0.869 | 0.888 | 0.904 | 0.843 | 0.966 | 0.932 | 0.855 | 1 |
| RSAD2_IFI44L_HERC6_IL1R2_FAM20A | 0.878 | 0.869 | 0.887 | 0.907 | 0.847 | 0.967 | 0.935 | 0.855 | 1 |
| OAS1_SIGLEC1_SLC1A2_IL1R2_OLAH | 0.878 | 0.869 | 0.887 | 0.913 | 0.858 | 0.968 | 0.963 | 0.925 | 1 |
| IFI27_ISG15_HERC6_SLC1A2_RETN | 0.878 | 0.869 | 0.888 | 0.910 | 0.848 | 0.971 | 0.943 | 0.878 | 1 |
| SIGLEC1_ISG15_HERC6_FAM20A_RETN | 0.878 | 0.868 | 0.888 | 0.897 | 0.827 | 0.968 | 0.955 | 0.890 | 1 |
| SIGLEC1_ISG15_FAM20A_RETN_MMP8 | 0.878 | 0.868 | 0.888 | 0.900 | 0.829 | 0.970 | 0.958 | 0.896 | 1 |
| IFIT1_IFI44L_SLC1A2_OLAH_FAM20A | 0.878 | 0.869 | 0.887 | 0.909 | 0.850 | 0.968 | 0.920 | 0.843 | 0.997 |
| RSAD2_SIGLEC1_HERC6_SLC1A2_FAM20A | 0.878 | 0.868 | 0.888 | 0.909 | 0.852 | 0.966 | 0.941 | 0.866 | 1 |
| RSAD2_IFI44L_SIGLEC1_HERC6_IL1R2 | 0.878 | 0.869 | 0.887 | 0.900 | 0.837 | 0.962 | 0.959 | 0.918 | 0.999 |
| OAS1_IFIT1_SIGLEC1_HERC6_RETN | 0.878 | 0.869 | 0.887 | 0.897 | 0.829 | 0.964 | 0.921 | 0.858 | 0.983 |
| SIGLEC1_SLC1A2_IL1R2_OLAH_RETN | 0.878 | 0.869 | 0.887 | 0.913 | 0.857 | 0.968 | 0.961 | 0.920 | 1 |
| OAS1_IFI44L_SIGLEC1_OLAH_FAM20A | 0.878 | 0.868 | 0.888 | 0.896 | 0.824 | 0.968 | 0.963 | 0.920 | 1 |
| IFI44L_HERC6_SLC1A2_IL1R2_FAM20A | 0.878 | 0.868 | 0.887 | 0.912 | 0.860 | 0.965 | 0.925 | 0.836 | 1 |
| IFI27_IFIT1_ISG15_HERC6_FAM20A | 0.878 | 0.868 | 0.888 | 0.904 | 0.842 | 0.966 | 0.929 | 0.846 | 1 |
| IFIT1_SIGLEC1_SLC1A2_OLAH_RETN | 0.878 | 0.869 | 0.887 | 0.915 | 0.860 | 0.970 | 0.948 | 0.897 | 0.999 |
| IFIT1_HERC6_IL1R2_FAM20A_RETN | 0.878 | 0.868 | 0.887 | 0.904 | 0.842 | 0.966 | 0.938 | 0.857 | 1 |
| IFI44L_SLC1A2_OLAH_FAM20A_RETN | 0.878 | 0.868 | 0.887 | 0.908 | 0.848 | 0.968 | 0.927 | 0.853 | 1 |
| IFI44L_HERC6_SLC1A2_OLAH_FAM20A | 0.878 | 0.868 | 0.888 | 0.912 | 0.855 | 0.969 | 0.932 | 0.853 | 1 |
| RSAD2_SIGLEC1_ISG15_HERC6_IL1R2 | 0.878 | 0.869 | 0.886 | 0.903 | 0.843 | 0.962 | 0.951 | 0.905 | 0.997 |
| IFI44L_SIGLEC1_FAM20A_RETN_MMP8 | 0.878 | 0.868 | 0.888 | 0.902 | 0.833 | 0.971 | 0.959 | 0.897 | 1 |
| SIGLEC1_HERC6_IL1R2_OLAH_MMP8 | 0.878 | 0.869 | 0.887 | 0.905 | 0.846 | 0.964 | 0.966 | 0.931 | 1 |
| RSAD2_IFIT1_HERC6_IL1R2_FAM20A | 0.878 | 0.868 | 0.887 | 0.904 | 0.842 | 0.966 | 0.935 | 0.853 | 1 |
| OAS1_SIGLEC1_HERC6_IL1R2_RETN | 0.878 | 0.868 | 0.887 | 0.901 | 0.837 | 0.965 | 0.957 | 0.913 | 1.000 |
| SIGLEC1_HERC6_SLC1A2_IL1R2_OLAH | 0.878 | 0.868 | 0.887 | 0.912 | 0.856 | 0.967 | 0.959 | 0.917 | 1 |
| SIGLEC1_ISG15_HERC6_SLC1A2_OLAH | 0.878 | 0.868 | 0.887 | 0.910 | 0.853 | 0.966 | 0.950 | 0.903 | 0.997 |
| OAS1_SIGLEC1_ISG15_SLC1A2_OLAH | 0.878 | 0.868 | 0.887 | 0.912 | 0.856 | 0.968 | 0.948 | 0.899 | 0.997 |
| RSAD2_OAS1_SIGLEC1_HERC6_FAM20A | 0.878 | 0.868 | 0.887 | 0.898 | 0.832 | 0.964 | 0.941 | 0.871 | 1 |
| IFI44L_SIGLEC1_OLAH_FAM20A_RETN | 0.877 | 0.868 | 0.887 | 0.896 | 0.823 | 0.968 | 0.962 | 0.918 | 1 |
| IFIT1_SIGLEC1_HERC6_IL1R2_OLAH | 0.877 | 0.869 | 0.886 | 0.907 | 0.848 | 0.965 | 0.950 | 0.904 | 0.996 |
| IFI44L_SIGLEC1_IL1R2_OLAH_MMP8 | 0.877 | 0.869 | 0.886 | 0.905 | 0.848 | 0.962 | 0.957 | 0.914 | 0.999 |
| OAS1_IFI44L_SIGLEC1_SLC1A2_OLAH | 0.877 | 0.868 | 0.887 | 0.914 | 0.859 | 0.969 | 0.949 | 0.900 | 0.998 |
| IFIT1_SIGLEC1_SLC1A2_FAM20A_RETN | 0.877 | 0.868 | 0.887 | 0.909 | 0.849 | 0.968 | 0.939 | 0.867 | 1 |
| IFIT1_IFI44L_HERC6_OLAH_FAM20A | 0.877 | 0.868 | 0.887 | 0.902 | 0.838 | 0.966 | 0.938 | 0.871 | 1 |
| SIGLEC1_ISG15_HERC6_IL1R2_MMP8 | 0.877 | 0.869 | 0.886 | 0.903 | 0.844 | 0.963 | 0.964 | 0.927 | 1 |
| OAS1_HERC6_IL1R2_FAM20A_RETN | 0.877 | 0.868 | 0.887 | 0.904 | 0.841 | 0.967 | 0.935 | 0.853 | 1 |
| OAS1_SIGLEC1_HERC6_SLC1A2_IL1R2 | 0.877 | 0.868 | 0.886 | 0.910 | 0.857 | 0.964 | 0.959 | 0.918 | 1.000 |
| IFIT1_SIGLEC1_HERC6_SLC1A2_IL1R2 | 0.877 | 0.869 | 0.886 | 0.908 | 0.854 | 0.963 | 0.961 | 0.922 | 1 |
| RSAD2_IFI27_IFIT1_HERC6_FAM20A | 0.877 | 0.867 | 0.887 | 0.907 | 0.846 | 0.967 | 0.934 | 0.853 | 1 |
| OAS1_SIGLEC1_ISG15_OLAH_FAM20A | 0.877 | 0.867 | 0.887 | 0.896 | 0.825 | 0.968 | 0.962 | 0.917 | 1 |
| IFI27_OAS1_SLC1A2_FAM20A_RETN | 0.877 | 0.868 | 0.887 | 0.910 | 0.848 | 0.971 | 0.938 | 0.856 | 1 |
| RSAD2_OAS1_IL1R2_OLAH_FAM20A | 0.877 | 0.868 | 0.886 | 0.901 | 0.835 | 0.967 | 0.940 | 0.866 | 1 |
| IFI44L_SIGLEC1_HERC6_IL1R2_RETN | 0.877 | 0.868 | 0.886 | 0.901 | 0.838 | 0.965 | 0.959 | 0.918 | 1.000 |
| IFI27_OAS1_IFIT1_ISG15_RETN | 0.877 | 0.868 | 0.887 | 0.904 | 0.841 | 0.968 | 0.950 | 0.887 | 1 |
| RSAD2_IFI27_SIGLEC1_HERC6_MMP8 | 0.877 | 0.868 | 0.886 | 0.901 | 0.842 | 0.959 | 0.909 | 0.834 | 0.983 |
| ISG15_HERC6_SLC1A2_IL1R2_FAM20A | 0.877 | 0.867 | 0.887 | 0.912 | 0.859 | 0.964 | 0.922 | 0.829 | 1 |
| SIGLEC1_SLC1A2_FAM20A_RETN_MMP8 | 0.877 | 0.867 | 0.887 | 0.903 | 0.839 | 0.967 | 0.953 | 0.882 | 1 |
| RSAD2_SIGLEC1_HERC6_IL1R2_OLAH | 0.877 | 0.868 | 0.886 | 0.906 | 0.847 | 0.965 | 0.955 | 0.912 | 0.999 |
| IFI27_IFI44L_ISG15_HERC6_FAM20A | 0.877 | 0.867 | 0.887 | 0.900 | 0.835 | 0.965 | 0.937 | 0.857 | 1 |
| IFI44L_IL1R2_OLAH_FAM20A_MMP8 | 0.877 | 0.868 | 0.886 | 0.903 | 0.842 | 0.964 | 0.947 | 0.883 | 1 |
| IFI27_SIGLEC1_SLC1A2_RETN_MMP8 | 0.877 | 0.867 | 0.887 | 0.910 | 0.849 | 0.970 | 0.955 | 0.901 | 1 |
| HERC6_SLC1A2_IL1R2_OLAH_FAM20A | 0.877 | 0.868 | 0.886 | 0.914 | 0.861 | 0.967 | 0.922 | 0.835 | 1 |
| OAS1_SIGLEC1_SLC1A2_OLAH_RETN | 0.877 | 0.867 | 0.887 | 0.913 | 0.857 | 0.969 | 0.949 | 0.898 | 0.999 |
| RSAD2_SIGLEC1_HERC6_SLC1A2_IL1R2 | 0.877 | 0.868 | 0.886 | 0.909 | 0.855 | 0.964 | 0.960 | 0.919 | 1 |
| IFI27_IFIT1_SLC1A2_FAM20A_RETN | 0.877 | 0.867 | 0.887 | 0.908 | 0.847 | 0.969 | 0.939 | 0.858 | 1 |
| IFI44L_SIGLEC1_SLC1A2_IL1R2_MMP8 | 0.877 | 0.869 | 0.885 | 0.909 | 0.855 | 0.963 | 0.966 | 0.930 | 1 |
| IFI27_OAS1_IFI44L_HERC6_FAM20A | 0.877 | 0.867 | 0.887 | 0.902 | 0.837 | 0.966 | 0.934 | 0.853 | 1 |
| RSAD2_SIGLEC1_ISG15_OLAH_FAM20A | 0.877 | 0.867 | 0.887 | 0.896 | 0.825 | 0.968 | 0.961 | 0.917 | 1 |
| SIGLEC1_ISG15_OLAH_FAM20A_RETN | 0.877 | 0.867 | 0.887 | 0.896 | 0.825 | 0.968 | 0.962 | 0.919 | 1 |
| SIGLEC1_ISG15_SLC1A2_OLAH_RETN | 0.877 | 0.868 | 0.886 | 0.914 | 0.858 | 0.970 | 0.950 | 0.901 | 0.999 |
| RSAD2_IFI27_OAS1_HERC6_FAM20A | 0.877 | 0.867 | 0.887 | 0.904 | 0.843 | 0.965 | 0.929 | 0.846 | 1 |
| SIGLEC1_HERC6_IL1R2_RETN_MMP8 | 0.877 | 0.867 | 0.886 | 0.901 | 0.835 | 0.966 | 0.970 | 0.937 | 1 |
| RSAD2_SIGLEC1_SLC1A2_OLAH_RETN | 0.877 | 0.868 | 0.886 | 0.913 | 0.856 | 0.969 | 0.950 | 0.901 | 0.999 |
| RSAD2_SIGLEC1_ISG15_SLC1A2_OLAH | 0.877 | 0.868 | 0.886 | 0.912 | 0.856 | 0.968 | 0.950 | 0.903 | 0.997 |
| IFI44L_SIGLEC1_HERC6_SLC1A2_OLAH | 0.877 | 0.867 | 0.886 | 0.912 | 0.856 | 0.967 | 0.953 | 0.909 | 0.998 |
| IFI27_IFI44L_SIGLEC1_HERC6_MMP8 | 0.877 | 0.868 | 0.886 | 0.897 | 0.836 | 0.959 | 0.908 | 0.833 | 0.982 |
| OAS1_SIGLEC1_HERC6_IL1R2_OLAH | 0.877 | 0.868 | 0.886 | 0.907 | 0.849 | 0.965 | 0.958 | 0.915 | 1.000 |
| RSAD2_SIGLEC1_HERC6_IL1R2_RETN | 0.877 | 0.867 | 0.886 | 0.901 | 0.836 | 0.965 | 0.954 | 0.911 | 0.998 |
| OAS1_SIGLEC1_FAM20A_RETN_MMP8 | 0.877 | 0.867 | 0.887 | 0.901 | 0.832 | 0.971 | 0.958 | 0.898 | 1 |
| IFI44L_ISG15_IL1R2_OLAH_FAM20A | 0.877 | 0.868 | 0.886 | 0.901 | 0.837 | 0.965 | 0.953 | 0.898 | 1 |
| OAS1_SIGLEC1_ISG15_HERC6_IL1R2 | 0.877 | 0.868 | 0.886 | 0.904 | 0.846 | 0.961 | 0.959 | 0.917 | 1 |
| IFI27_ISG15_SLC1A2_FAM20A_RETN | 0.877 | 0.867 | 0.887 | 0.904 | 0.841 | 0.968 | 0.926 | 0.839 | 1 |
| IFIT1_SIGLEC1_HERC6_OLAH_RETN | 0.877 | 0.867 | 0.886 | 0.900 | 0.835 | 0.965 | 0.938 | 0.885 | 0.991 |
| RSAD2_HERC6_IL1R2_FAM20A_RETN | 0.877 | 0.867 | 0.886 | 0.905 | 0.842 | 0.967 | 0.932 | 0.848 | 1 |
| HERC6_IL1R2_FAM20A_RETN_MMP8 | 0.877 | 0.867 | 0.886 | 0.905 | 0.844 | 0.967 | 0.925 | 0.836 | 1 |
| RSAD2_IFI27_SLC1A2_FAM20A_RETN | 0.876 | 0.867 | 0.886 | 0.910 | 0.848 | 0.971 | 0.937 | 0.855 | 1 |
| IFIT1_IL1R2_OLAH_FAM20A_MMP8 | 0.876 | 0.867 | 0.886 | 0.905 | 0.844 | 0.967 | 0.949 | 0.883 | 1 |
| IFI44L_SIGLEC1_SLC1A2_IL1R2_RETN | 0.876 | 0.868 | 0.885 | 0.910 | 0.855 | 0.965 | 0.962 | 0.922 | 1 |
| RSAD2_SLC1A2_IL1R2_OLAH_FAM20A | 0.876 | 0.868 | 0.885 | 0.911 | 0.855 | 0.968 | 0.932 | 0.851 | 1 |
| RSAD2_SIGLEC1_HERC6_FAM20A_MMP8 | 0.876 | 0.867 | 0.886 | 0.898 | 0.832 | 0.964 | 0.942 | 0.873 | 1 |
| OAS1_SIGLEC1_OLAH_FAM20A_RETN | 0.876 | 0.866 | 0.886 | 0.896 | 0.824 | 0.968 | 0.962 | 0.918 | 1 |
| SIGLEC1_HERC6_SLC1A2_IL1R2_MMP8 | 0.876 | 0.868 | 0.885 | 0.907 | 0.852 | 0.962 | 0.963 | 0.925 | 1 |
| OAS1_IFIT1_IL1R2_OLAH_FAM20A | 0.876 | 0.867 | 0.885 | 0.901 | 0.835 | 0.967 | 0.945 | 0.873 | 1 |
| IFI27_HERC6_SLC1A2_RETN_MMP8 | 0.876 | 0.866 | 0.886 | 0.908 | 0.847 | 0.970 | 0.935 | 0.864 | 1 |
| IFI44L_SIGLEC1_ISG15_IL1R2_MMP8 | 0.876 | 0.868 | 0.884 | 0.904 | 0.846 | 0.961 | 0.957 | 0.914 | 0.999 |
| IFI44L_SIGLEC1_ISG15_SLC1A2_IL1R2 | 0.876 | 0.868 | 0.884 | 0.907 | 0.851 | 0.963 | 0.963 | 0.925 | 1 |
| IFIT1_SLC1A2_IL1R2_FAM20A_MMP8 | 0.876 | 0.867 | 0.885 | 0.909 | 0.854 | 0.964 | 0.936 | 0.857 | 1 |
| RSAD2_SIGLEC1_FAM20A_RETN_MMP8 | 0.876 | 0.866 | 0.886 | 0.902 | 0.835 | 0.970 | 0.957 | 0.896 | 1 |
| IFIT1_HERC6_SLC1A2_IL1R2_FAM20A | 0.876 | 0.866 | 0.885 | 0.912 | 0.858 | 0.966 | 0.932 | 0.848 | 1 |
| RSAD2_SIGLEC1_SLC1A2_IL1R2_MMP8 | 0.876 | 0.867 | 0.884 | 0.909 | 0.854 | 0.963 | 0.967 | 0.933 | 1 |
| RSAD2_IFI44L_SIGLEC1_OLAH_FAM20A | 0.876 | 0.866 | 0.886 | 0.897 | 0.826 | 0.968 | 0.963 | 0.921 | 1 |
| OAS1_ISG15_IL1R2_OLAH_FAM20A | 0.876 | 0.867 | 0.885 | 0.901 | 0.835 | 0.967 | 0.940 | 0.866 | 1 |
| IFI27_IFIT1_IFI44L_FAM20A_MMP8_{—} | 0.876 | 0.866 | 0.886 | 0.899 | 0.832 | 0.966 | 0.934 | 0.854 | 1 |
| SIGLEC1_SLC1A2_OLAH_RETN_MMP8 | 0.876 | 0.866 | 0.885 | 0.909 | 0.851 | 0.967 | 0.962 | 0.923 | 1 |
| RSAD2_IFI44L_SLC1A2_OLAH_FAM20A | 0.876 | 0.866 | 0.885 | 0.908 | 0.849 | 0.968 | 0.926 | 0.852 | 1 |
| OAS1_HERC6_SLC1A2_IL1R2_FAM20A | 0.876 | 0.867 | 0.885 | 0.911 | 0.857 | 0.965 | 0.923 | 0.832 | 1 |
| RSAD2_HERC6_SLC1A2_IL1R2_FAM20A | 0.876 | 0.867 | 0.885 | 0.911 | 0.857 | 0.964 | 0.927 | 0.837 | 1 |
| OAS1_IFIT1_SIGLEC1_IL1R2_OLAH | 0.876 | 0.867 | 0.885 | 0.901 | 0.838 | 0.965 | 0.957 | 0.914 | 0.999 |
| OAS1_SLC1A2_IL1R2_OLAH_FAM20A | 0.876 | 0.867 | 0.885 | 0.911 | 0.854 | 0.968 | 0.927 | 0.844 | 1 |
| IFI27_OAS1_IFIT1_HERC6_FAM20A | 0.876 | 0.866 | 0.886 | 0.903 | 0.841 | 0.965 | 0.935 | 0.854 | 1 |
| IFIT1_SIGLEC1_HERC6_OLAH_MMP8 | 0.876 | 0.866 | 0.885 | 0.899 | 0.837 | 0.961 | 0.936 | 0.881 | 0.991 |
| OAS1_SIGLEC1_HERC6_SLC1A2_FAM20A | 0.876 | 0.866 | 0.885 | 0.908 | 0.852 | 0.965 | 0.943 | 0.870 | 1 |
| OAS1_HERC6_SLC1A2_OLAH_FAM20A | 0.876 | 0.866 | 0.885 | 0.910 | 0.854 | 0.966 | 0.928 | 0.846 | 1 |
| IFIT1_SIGLEC1_ISG15_SLC1A2_IL1R2 | 0.876 | 0.868 | 0.884 | 0.908 | 0.853 | 0.964 | 0.964 | 0.927 | 1 |
| ISG15_SLC1A2_IL1R2_OLAH_FAM20A | 0.876 | 0.866 | 0.885 | 0.908 | 0.850 | 0.965 | 0.924 | 0.844 | 1 |
| IFI44L_SIGLEC1_SLC1A2_FAM20A_MMP8 | 0.876 | 0.866 | 0.885 | 0.901 | 0.837 | 0.966 | 0.952 | 0.880 | 1 |
| RSAD2_IFI44L_SIGLEC1_SLC1A2_OLAH | 0.876 | 0.866 | 0.885 | 0.912 | 0.857 | 0.968 | 0.949 | 0.901 | 0.997 |
| IFI44L_SIGLEC1_ISG15_FAM20A_RETN | 0.876 | 0.866 | 0.885 | 0.894 | 0.823 | 0.966 | 0.955 | 0.898 | 1 |
| SIGLEC1_HERC6_SLC1A2_FAM20A_RETN | 0.875 | 0.865 | 0.886 | 0.903 | 0.839 | 0.966 | 0.950 | 0.876 | 1 |
| IFI44L_SIGLEC1_HERC6_SLC1A2_FAM20A | 0.875 | 0.866 | 0.885 | 0.904 | 0.842 | 0.966 | 0.953 | 0.880 | 1 |
| SIGLEC1_ISG15_HERC6_IL1R2_OLAH | 0.875 | 0.866 | 0.885 | 0.903 | 0.842 | 0.964 | 0.959 | 0.918 | 1.000 |
| SIGLEC1_ISG15_HERC6_SLC1A2_IL1R2 | 0.875 | 0.867 | 0.884 | 0.908 | 0.853 | 0.963 | 0.962 | 0.923 | 1 |
| OAS1_IFI44L_SLC1A2_OLAH_FAM20A | 0.875 | 0.866 | 0.885 | 0.909 | 0.849 | 0.969 | 0.927 | 0.852 | 1 |
| RSAD2_HERC6_SLC1A2_OLAH_FAM20A | 0.875 | 0.865 | 0.885 | 0.910 | 0.854 | 0.966 | 0.929 | 0.848 | 1 |
| IFIT1_ISG15_IL1R2_FAM20A_MMP8 | 0.875 | 0.866 | 0.885 | 0.907 | 0.846 | 0.969 | 0.941 | 0.867 | 1 |
| ISG15_HERC6_SLC1A2_OLAH_FAM20A | 0.875 | 0.866 | 0.885 | 0.911 | 0.855 | 0.967 | 0.932 | 0.852 | 1 |
| IFIT1_SIGLEC1_IL1R2_OLAH_MMP8 | 0.875 | 0.867 | 0.884 | 0.905 | 0.848 | 0.962 | 0.963 | 0.925 | 1 |
| SIGLEC1_ISG15_SLC1A2_IL1R2_MMP8 | 0.875 | 0.868 | 0.883 | 0.910 | 0.856 | 0.964 | 0.966 | 0.930 | 1 |
| IFIT1_SIGLEC1_SLC1A2_IL1R2_MMP8 | 0.875 | 0.867 | 0.884 | 0.910 | 0.855 | 0.964 | 0.966 | 0.930 | 1 |
| RSAD2_IFI27_OAS1_IFI44L_RETN | 0.875 | 0.866 | 0.885 | 0.905 | 0.840 | 0.969 | 0.953 | 0.892 | 1 |
| IFI27_OAS1_IFI44L_ISG15_RETN | 0.875 | 0.866 | 0.885 | 0.906 | 0.843 | 0.969 | 0.954 | 0.896 | 1 |
| IFI44L_ISG15_IL1R2_FAM20A_MMP8 | 0.875 | 0.867 | 0.884 | 0.904 | 0.844 | 0.964 | 0.937 | 0.858 | 1 |
| IFI27_IFI44L_ISG15_FAM20A_MMP8 | 0.875 | 0.865 | 0.885 | 0.898 | 0.831 | 0.965 | 0.938 | 0.860 | 1 |
| RSAD2_OAS1_IL1R2_FAM20A_MMP8 | 0.875 | 0.866 | 0.884 | 0.903 | 0.840 | 0.965 | 0.934 | 0.853 | 1 |
| IFIT1_ISG15_SLC1A2_IL1R2_FAM20A | 0.875 | 0.866 | 0.884 | 0.904 | 0.846 | 0.963 | 0.935 | 0.857 | 1 |
| RSAD2_OAS1_SIGLEC1_SLC1A2_FAM20A | 0.875 | 0.866 | 0.885 | 0.903 | 0.840 | 0.966 | 0.947 | 0.874 | 1 |
| RSAD2_OAS1_SIGLEC1_FAM20A_RETN | 0.875 | 0.866 | 0.885 | 0.895 | 0.824 | 0.966 | 0.955 | 0.897 | 1 |
| SIGLEC1_ISG15_IL1R2_OLAH_MMP8 | 0.875 | 0.867 | 0.884 | 0.905 | 0.847 | 0.963 | 0.963 | 0.925 | 1 |
| RSAD2_IFI27_OAS1_IFI44L_FAM20A | 0.875 | 0.865 | 0.885 | 0.901 | 0.835 | 0.968 | 0.938 | 0.862 | 1 |
| RSAD2_SIGLEC1_SLC1A2_FAM20A_MMP8 | 0.875 | 0.866 | 0.885 | 0.902 | 0.839 | 0.965 | 0.949 | 0.878 | 1 |
| IFI44L_SIGLEC1_IL1R2_RETN_MMP8 | 0.875 | 0.866 | 0.885 | 0.902 | 0.839 | 0.964 | 0.962 | 0.924 | 1 |
| OAS1_IFIT1_SIGLEC1_HERC6_OLAH | 0.875 | 0.866 | 0.884 | 0.900 | 0.839 | 0.962 | 0.937 | 0.883 | 0.991 |
| IFI44L_SIGLEC1_ISG15_IL1R2_OLAH | 0.875 | 0.867 | 0.884 | 0.902 | 0.841 | 0.963 | 0.958 | 0.915 | 1 |
| OAS1_IFI44L_SIGLEC1_IL1R2_MMP8 | 0.875 | 0.867 | 0.884 | 0.902 | 0.842 | 0.962 | 0.957 | 0.914 | 0.999 |
| SIGLEC1_ISG15_SLC1A2_FAM20A_MMP8 | 0.875 | 0.865 | 0.885 | 0.900 | 0.836 | 0.964 | 0.952 | 0.880 | 1 |
| HERC6_SLC1A2_OLAH_FAM20A_MMP8 | 0.875 | 0.866 | 0.885 | 0.911 | 0.856 | 0.966 | 0.928 | 0.846 | 1 |
| IFI27_OAS1_ISG15_HERC6_MMP8 | 0.875 | 0.867 | 0.884 | 0.901 | 0.844 | 0.958 | 0.900 | 0.822 | 0.978 |
| OAS1_IFIT1_IFI44L_SIGLEC1_IL1R2 | 0.875 | 0.866 | 0.884 | 0.899 | 0.835 | 0.962 | 0.955 | 0.913 | 0.998 |
| RSAD2_IFI27_OAS1_IFIT1_RETN | 0.875 | 0.866 | 0.885 | 0.905 | 0.841 | 0.968 | 0.949 | 0.885 | 1 |
| OAS1_IFIT1_SIGLEC1_IL1R2_MMP8 | 0.875 | 0.866 | 0.884 | 0.901 | 0.842 | 0.961 | 0.963 | 0.925 | 1 |
| RSAD2_SIGLEC1_IL1R2_OLAH_MMP8 | 0.875 | 0.866 | 0.884 | 0.905 | 0.847 | 0.963 | 0.963 | 0.925 | 1 |
| SIGLEC1_ISG15_SLC1A2_IL1R2_RETN | 0.875 | 0.867 | 0.883 | 0.909 | 0.854 | 0.964 | 0.965 | 0.927 | 1 |
| OAS1_IFIT1_SIGLEC1_SLC1A2_IL1R2 | 0.875 | 0.866 | 0.884 | 0.908 | 0.853 | 0.963 | 0.963 | 0.925 | 1 |
| SIGLEC1_ISG15_SLC1A2_FAM20A_RETN | 0.875 | 0.865 | 0.885 | 0.902 | 0.839 | 0.966 | 0.948 | 0.875 | 1 |
| IFI44L_SIGLEC1_ISG15_SLC1A2_FAM20A | 0.875 | 0.866 | 0.884 | 0.901 | 0.837 | 0.965 | 0.949 | 0.877 | 1 |
| RSAD2_IFI27_IFI44L_ISG15_FAM20A | 0.875 | 0.865 | 0.885 | 0.903 | 0.836 | 0.970 | 0.943 | 0.870 | 1 |
| IFIT1_IFI44L_SIGLEC1_IL1R2_MMP8 | 0.875 | 0.866 | 0.884 | 0.901 | 0.842 | 0.961 | 0.960 | 0.919 | 1 |
| IFI27_OAS1_IFIT1_IFI44L_RETN | 0.875 | 0.865 | 0.885 | 0.906 | 0.842 | 0.969 | 0.948 | 0.882 | 1 |
| IFIT1_SIGLEC1_SLC1A2_IL1R2_RETN | 0.875 | 0.866 | 0.884 | 0.910 | 0.855 | 0.965 | 0.962 | 0.922 | 1 |
| RSAD2_SIGLEC1_OLAH_FAM20A_RETN | 0.875 | 0.865 | 0.885 | 0.897 | 0.825 | 0.969 | 0.961 | 0.917 | 1 |
| IFI27_IFI44L_ISG15_RETN_MMP8 | 0.875 | 0.865 | 0.885 | 0.904 | 0.841 | 0.967 | 0.953 | 0.896 | 1 |
| RSAD2_OAS1_SIGLEC1_FAM20A_MMP8 | 0.875 | 0.865 | 0.885 | 0.895 | 0.824 | 0.966 | 0.950 | 0.885 | 1 |
| RSAD2_IFI44L_SIGLEC1_IL1R2_MMP8 | 0.875 | 0.866 | 0.883 | 0.900 | 0.839 | 0.962 | 0.960 | 0.919 | 1 |
| SIGLEC1_HERC6_SLC1A2_IL1R2_RETN | 0.875 | 0.866 | 0.884 | 0.909 | 0.853 | 0.965 | 0.960 | 0.919 | 1 |
| IFI44L_SIGLEC1_ISG15_HERC6_FAM20A | 0.875 | 0.865 | 0.885 | 0.897 | 0.830 | 0.965 | 0.953 | 0.882 | 1 |
| OAS1_SIGLEC1_SLC1A2_IL1R2_MMP8 | 0.875 | 0.866 | 0.883 | 0.910 | 0.857 | 0.964 | 0.966 | 0.930 | 1 |
| RSAD2_IFI44L_SIGLEC1_HERC6_FAM20A | 0.875 | 0.865 | 0.884 | 0.900 | 0.835 | 0.965 | 0.941 | 0.871 | 1 |
| OAS1_IFIT1_ISG15_IL1R2_FAM20A | 0.875 | 0.865 | 0.884 | 0.900 | 0.835 | 0.965 | 0.942 | 0.868 | 1 |
| IFI44L_SLC1A2_IL1R2_FAM20A_RETN | 0.875 | 0.866 | 0.884 | 0.909 | 0.855 | 0.963 | 0.926 | 0.840 | 1 |
| IFIT1_HERC6_SLC1A2_OLAH_FAM20A | 0.875 | 0.865 | 0.885 | 0.910 | 0.854 | 0.966 | 0.930 | 0.849 | 1 |
| IFIT1_SIGLEC1_ISG15_HERC6_RETN | 0.875 | 0.865 | 0.884 | 0.896 | 0.828 | 0.963 | 0.935 | 0.880 | 0.989 |
| IFI44L_SIGLEC1_HERC6_SLC1A2_IL1R2 | 0.875 | 0.866 | 0.884 | 0.908 | 0.853 | 0.963 | 0.960 | 0.920 | 1.000 |
| RSAD2_SIGLEC1_SLC1A2_IL1R2_RETN | 0.875 | 0.866 | 0.884 | 0.910 | 0.855 | 0.965 | 0.965 | 0.928 | 1 |
| HERC6_SLC1A2_IL1R2_FAM20A_RETN | 0.875 | 0.865 | 0.884 | 0.912 | 0.858 | 0.966 | 0.918 | 0.824 | 1 |
| RSAD2_OAS1_SIGLEC1_IL1R2_MMP8 | 0.875 | 0.866 | 0.884 | 0.903 | 0.844 | 0.962 | 0.965 | 0.929 | 1 |
| SIGLEC1_HERC6_SLC1A2_OLAH_RETN | 0.875 | 0.865 | 0.885 | 0.910 | 0.851 | 0.968 | 0.958 | 0.915 | 1.000 |
| RSAD2_OAS1_SIGLEC1_ISG15_FAM20A | 0.875 | 0.865 | 0.884 | 0.897 | 0.826 | 0.968 | 0.952 | 0.889 | 1 |
| SIGLEC1_SLC1A2_IL1R2_RETN_MMP8 | 0.875 | 0.866 | 0.883 | 0.912 | 0.858 | 0.966 | 0.968 | 0.935 | 1 |
| IFIT1_IFI44L_SLC1A2_IL1R2_FAM20A | 0.874 | 0.866 | 0.883 | 0.909 | 0.854 | 0.964 | 0.924 | 0.837 | 1 |
| OAS1_SIGLEC1_IL1R2_OLAH_MMP8 | 0.874 | 0.866 | 0.883 | 0.903 | 0.844 | 0.962 | 0.964 | 0.927 | 1 |
| OAS1_IFI44L_IL1R2_FAM20A_MMP8 | 0.874 | 0.865 | 0.884 | 0.904 | 0.842 | 0.966 | 0.938 | 0.858 | 1 |
| IFI27_HERC6_SLC1A2_FAM20A_MMP8 | 0.874 | 0.864 | 0.885 | 0.908 | 0.849 | 0.966 | 0.929 | 0.846 | 1 |
| IFIT1_HERC6_OLAH_FAM20A_RETN | 0.874 | 0.865 | 0.884 | 0.903 | 0.840 | 0.966 | 0.943 | 0.877 | 1 |
| OAS1_IFI44L_IL1R2_OLAH_FAM20A | 0.874 | 0.865 | 0.884 | 0.900 | 0.834 | 0.967 | 0.948 | 0.880 | 1 |
| RSAD2_SIGLEC1_ISG15_SLC1A2_IL1R2 | 0.874 | 0.866 | 0.883 | 0.909 | 0.854 | 0.964 | 0.964 | 0.927 | 1 |
| RSAD2_IL1R2_OLAH_FAM20A_MMP8 | 0.874 | 0.865 | 0.883 | 0.902 | 0.839 | 0.964 | 0.941 | 0.868 | 1 |
| RSAD2_IFIT1_SIGLEC1_SLC1A2_IL1R2 | 0.874 | 0.866 | 0.883 | 0.906 | 0.850 | 0.962 | 0.964 | 0.927 | 1 |
| IFIT1_IFI44L_IL1R2_FAM20A_MMP8 | 0.874 | 0.865 | 0.883 | 0.903 | 0.840 | 0.966 | 0.943 | 0.869 | 1 |
| IFI44L_SIGLEC1_SLC1A2_FAM20A_RETN | 0.874 | 0.865 | 0.884 | 0.903 | 0.838 | 0.967 | 0.948 | 0.875 | 1 |
| IFI27_IFIT1_IFI44L_ISG15_RETN | 0.874 | 0.864 | 0.884 | 0.904 | 0.840 | 0.967 | 0.953 | 0.896 | 1 |
| SIGLEC1_ISG15_IL1R2_RETN_MMP8 | 0.874 | 0.865 | 0.883 | 0.901 | 0.837 | 0.964 | 0.971 | 0.938 | 1 |
| RSAD2_OAS1_IFI44L_IL1R2_FAM20A | 0.874 | 0.865 | 0.883 | 0.901 | 0.836 | 0.965 | 0.941 | 0.867 | 1 |
| IFI27_OAS1_SIGLEC1_SLC1A2_MMP8 | 0.874 | 0.865 | 0.883 | 0.904 | 0.845 | 0.963 | 0.934 | 0.868 | 1.000 |
| OAS1_SIGLEC1_SLC1A2_IL1R2_RETN | 0.874 | 0.865 | 0.883 | 0.910 | 0.855 | 0.965 | 0.962 | 0.922 | 1 |
| RSAD2_IFI27_IFI44L_FAM20A_MMP8 | 0.874 | 0.864 | 0.885 | 0.899 | 0.832 | 0.966 | 0.935 | 0.857 | 1 |
| RSAD2_OAS1_IFIT1_IL1R2_FAM20A | 0.874 | 0.865 | 0.883 | 0.901 | 0.836 | 0.966 | 0.941 | 0.867 | 1 |
| RSAD2_SIGLEC1_ISG15_SLC1A2_FAM20A | 0.874 | 0.865 | 0.884 | 0.902 | 0.839 | 0.965 | 0.947 | 0.877 | 1 |
| SIGLEC1_HERC6_SLC1A2_FAM20A_MMP8 | 0.874 | 0.864 | 0.884 | 0.901 | 0.838 | 0.964 | 0.954 | 0.881 | 1 |
| HERC6_SLC1A2_IL1R2_FAM20A_MMP8 | 0.874 | 0.865 | 0.883 | 0.913 | 0.862 | 0.964 | 0.917 | 0.819 | 1 |
| OAS1_IFI44L_SIGLEC1_IL1R2_OLAH | 0.874 | 0.865 | 0.883 | 0.901 | 0.837 | 0.965 | 0.951 | 0.905 | 0.997 |
| IFIT1_SIGLEC1_ISG15_HERC6_OLAH | 0.874 | 0.865 | 0.883 | 0.901 | 0.839 | 0.963 | 0.941 | 0.890 | 0.993 |
| OAS1_SIGLEC1_ISG15_HERC6_FAM20A | 0.874 | 0.865 | 0.884 | 0.897 | 0.831 | 0.964 | 0.948 | 0.884 | 1 |
| IFIT1_IFI44L_SIGLEC1_HERC6_OLAH | 0.874 | 0.865 | 0.883 | 0.899 | 0.837 | 0.961 | 0.937 | 0.883 | 0.991 |
| HERC6_OLAH_FAM20A_RETN_MMP8 | 0.874 | 0.864 | 0.884 | 0.900 | 0.835 | 0.965 | 0.949 | 0.885 | 1 |
| SIGLEC1_ISG15_HERC6_SLC1A2_FAM20A | 0.874 | 0.864 | 0.884 | 0.901 | 0.838 | 0.964 | 0.952 | 0.879 | 1 |
| IFI44L_SIGLEC1_HERC6_FAM20A_MMP8 | 0.874 | 0.864 | 0.884 | 0.898 | 0.831 | 0.966 | 0.953 | 0.882 | 1 |
| IFIT1_SLC1A2_OLAH_FAM20A_MMP8 | 0.874 | 0.865 | 0.883 | 0.906 | 0.847 | 0.965 | 0.939 | 0.869 | 1 |
| OAS1_SIGLEC1_ISG15_SLC1A2_IL1R2 | 0.874 | 0.865 | 0.883 | 0.908 | 0.853 | 0.964 | 0.964 | 0.927 | 1 |
| IFIT1_ISG15_HERC6_OLAH_FAM20A | 0.874 | 0.864 | 0.884 | 0.900 | 0.837 | 0.964 | 0.941 | 0.872 | 1 |
| IFIT1_IFI44L_SIGLEC1_HERC6_RETN | 0.874 | 0.865 | 0.883 | 0.894 | 0.824 | 0.964 | 0.925 | 0.865 | 0.985 |
| RSAD2_OAS1_SIGLEC1_SLC1A2_IL1R2 | 0.874 | 0.865 | 0.883 | 0.908 | 0.852 | 0.963 | 0.965 | 0.926 | 1 |
| RSAD2_OAS1_SLC1A2_OLAH_FAM20A | 0.874 | 0.864 | 0.883 | 0.911 | 0.853 | 0.969 | 0.935 | 0.862 | 1 |
| IFI27_OAS1_IFIT1_HERC6_MMP8 | 0.874 | 0.865 | 0.883 | 0.899 | 0.840 | 0.957 | 0.892 | 0.811 | 0.974 |
| HERC6_SLC1A2_OLAH_FAM20A_RETN | 0.874 | 0.864 | 0.884 | 0.910 | 0.854 | 0.966 | 0.928 | 0.847 | 1 |
| IFI27_IFI44L_SLC1A2_FAM20A_MMP8 | 0.874 | 0.864 | 0.884 | 0.900 | 0.836 | 0.965 | 0.937 | 0.857 | 1 |
| RSAD2_OAS1_SIGLEC1_IL1R2_OLAH | 0.874 | 0.864 | 0.883 | 0.900 | 0.837 | 0.963 | 0.960 | 0.919 | 1 |
| OAS1_IL1R2_OLAH_FAM20A_RETN | 0.874 | 0.864 | 0.883 | 0.900 | 0.834 | 0.965 | 0.932 | 0.853 | 1 |
| OAS1_SLC1A2_OLAH_FAM20A_MMP8 | 0.874 | 0.864 | 0.883 | 0.909 | 0.852 | 0.966 | 0.934 | 0.858 | 1 |
| OAS1_IFIT1_IFI44L_IL1R2_FAM20A | 0.874 | 0.865 | 0.883 | 0.899 | 0.834 | 0.965 | 0.941 | 0.867 | 1 |
| OAS1_IFIT1_SIGLEC1_ISG15_IL1R2 | 0.874 | 0.865 | 0.882 | 0.899 | 0.836 | 0.962 | 0.955 | 0.913 | 0.998 |
| IFIT1_HERC6_OLAH_FAM20A_MMP8 | 0.874 | 0.864 | 0.884 | 0.899 | 0.835 | 0.963 | 0.943 | 0.876 | 1 |
| RSAD2_OAS1_ISG15_IL1R2_FAM20A | 0.874 | 0.864 | 0.883 | 0.897 | 0.831 | 0.963 | 0.936 | 0.857 | 1 |
| IFIT1_ISG15_HERC6_FAM20A_RETN | 0.874 | 0.864 | 0.883 | 0.899 | 0.834 | 0.963 | 0.940 | 0.864 | 1 |
| OAS1_SIGLEC1_ISG15_FAM20A_RETN | 0.874 | 0.864 | 0.884 | 0.894 | 0.822 | 0.966 | 0.955 | 0.898 | 1 |
| OAS1_SIGLEC1_ISG15_HERC6_RETN | 0.874 | 0.863 | 0.884 | 0.896 | 0.829 | 0.964 | 0.953 | 0.907 | 1.000 |
| RSAD2_IFIT1_IFI44L_SIGLEC1_IL1R2 | 0.874 | 0.865 | 0.882 | 0.896 | 0.831 | 0.961 | 0.955 | 0.913 | 0.998 |
| IFI44L_HERC6_OLAH_FAM20A_MMP8 | 0.874 | 0.864 | 0.884 | 0.899 | 0.833 | 0.964 | 0.947 | 0.881 | 1 |
| ISG15_HERC6_OLAH_FAM20A_RETN | 0.874 | 0.864 | 0.883 | 0.899 | 0.833 | 0.965 | 0.950 | 0.887 | 1 |
| RSAD2_OAS1_IFI44L_SIGLEC1_FAM20A | 0.873 | 0.864 | 0.883 | 0.897 | 0.826 | 0.968 | 0.953 | 0.892 | 1 |
| IFI44L_SIGLEC1_IL1R2_OLAH_RETN | 0.873 | 0.864 | 0.883 | 0.899 | 0.834 | 0.963 | 0.958 | 0.914 | 1 |
| IFIT1_SIGLEC1_ISG15_IL1R2_OLAH | 0.873 | 0.865 | 0.882 | 0.903 | 0.842 | 0.964 | 0.960 | 0.918 | 1 |
| RSAD2_SLC1A2_OLAH_FAM20A_MMP8 | 0.873 | 0.864 | 0.883 | 0.905 | 0.847 | 0.964 | 0.935 | 0.863 | 1 |
| SIGLEC1_IL1R2_OLAH_RETN_MMP8 | 0.873 | 0.864 | 0.883 | 0.901 | 0.838 | 0.964 | 0.968 | 0.935 | 1 |
| RSAD2_OAS1_IFIT1_SIGLEC1_IL1R2 | 0.873 | 0.864 | 0.882 | 0.897 | 0.835 | 0.960 | 0.961 | 0.922 | 1 |
| RSAD2_IFIT1_SIGLEC1_HERC6_RETN | 0.873 | 0.864 | 0.883 | 0.893 | 0.824 | 0.963 | 0.923 | 0.862 | 0.984 |
| RSAD2_IFIT1_IL1R2_FAM20A_MMP8 | 0.873 | 0.864 | 0.882 | 0.905 | 0.843 | 0.968 | 0.943 | 0.870 | 1 |
| OAS1_IFIT1_SLC1A2_IL1R2_FAM20A | 0.873 | 0.864 | 0.882 | 0.907 | 0.850 | 0.965 | 0.936 | 0.855 | 1 |
| IFI44L_HERC6_OLAH_FAM20A_RETN | 0.873 | 0.864 | 0.883 | 0.901 | 0.835 | 0.967 | 0.948 | 0.884 | 1 |
| IFI44L_SIGLEC1_ISG15_IL1R2_RETN | 0.873 | 0.865 | 0.882 | 0.900 | 0.837 | 0.963 | 0.960 | 0.917 | 1 |
| IFIT1_IFI44L_SIGLEC1_ISG15_IL1R2 | 0.873 | 0.865 | 0.882 | 0.901 | 0.840 | 0.962 | 0.959 | 0.918 | 1.000 |
| IFIT1_ISG15_IL1R2_OLAH_FAM20A | 0.873 | 0.864 | 0.883 | 0.900 | 0.835 | 0.964 | 0.953 | 0.896 | 1 |
| IFI44L_ISG15_HERC6_OLAH_FAM20A | 0.873 | 0.863 | 0.883 | 0.897 | 0.830 | 0.963 | 0.947 | 0.882 | 1 |
| SIGLEC1_ISG15_HERC6_IL1R2_RETN | 0.873 | 0.864 | 0.883 | 0.898 | 0.831 | 0.964 | 0.962 | 0.923 | 1 |
| OAS1_IFIT1_IL1R2_FAM20A_RETN | 0.873 | 0.864 | 0.883 | 0.900 | 0.835 | 0.966 | 0.945 | 0.871 | 1 |
| OAS1_IFIT1_SLC1A2_OLAH_FAM20A | 0.873 | 0.864 | 0.883 | 0.909 | 0.850 | 0.968 | 0.935 | 0.861 | 1 |
| RSAD2_SIGLEC1_IL1R2_RETN_MMP8 | 0.873 | 0.864 | 0.883 | 0.899 | 0.835 | 0.964 | 0.972 | 0.940 | 1 |
| OAS1_IFI44L_SLC1A2_IL1R2_FAM20A | 0.873 | 0.864 | 0.882 | 0.908 | 0.851 | 0.964 | 0.925 | 0.837 | 1 |
| RSAD2_IFIT1_SIGLEC1_IL1R2_MMP8 | 0.873 | 0.864 | 0.882 | 0.901 | 0.841 | 0.961 | 0.966 | 0.931 | 1 |
| IFI27_OAS1_IFIT1_RETN_MMP8 | 0.873 | 0.863 | 0.883 | 0.904 | 0.841 | 0.968 | 0.949 | 0.887 | 1 |
| IFIT1_IFI44L_SIGLEC1_SLC1A2_IL1R2 | 0.873 | 0.865 | 0.882 | 0.907 | 0.851 | 0.963 | 0.964 | 0.927 | 1 |
| OAS1_IFIT1_HERC6_OLAH_FAM20A | 0.873 | 0.863 | 0.883 | 0.898 | 0.833 | 0.963 | 0.940 | 0.871 | 1 |
| OAS1_SIGLEC1_IL1R2_RETN_MMP8 | 0.873 | 0.864 | 0.883 | 0.899 | 0.834 | 0.963 | 0.970 | 0.936 | 1 |
| IFI44L_SIGLEC1_ISG15_FAM20A_MMP8 | 0.873 | 0.863 | 0.883 | 0.890 | 0.818 | 0.963 | 0.957 | 0.895 | 1 |
| SIGLEC1_HERC6_IL1R2_OLAH_RETN | 0.873 | 0.863 | 0.883 | 0.900 | 0.835 | 0.966 | 0.960 | 0.919 | 1 |
| OAS1_ISG15_IL1R2_FAM20A_MMP8 | 0.873 | 0.864 | 0.882 | 0.903 | 0.841 | 0.966 | 0.934 | 0.853 | 1 |
| OAS1_IFIT1_SIGLEC1_IL1R2_RETN | 0.873 | 0.864 | 0.882 | 0.896 | 0.829 | 0.962 | 0.958 | 0.916 | 0.999 |
| RSAD2_IFI44L_SIGLEC1_IL1R2_OLAH | 0.873 | 0.864 | 0.882 | 0.899 | 0.834 | 0.964 | 0.961 | 0.921 | 1 |
| OAS1_IFI44L_SIGLEC1_SLC1A2_IL1R2 | 0.873 | 0.864 | 0.882 | 0.907 | 0.852 | 0.963 | 0.963 | 0.925 | 1 |
| RSAD2_OAS1_HERC6_OLAH_FAM20A | 0.873 | 0.863 | 0.883 | 0.901 | 0.836 | 0.965 | 0.938 | 0.870 | 1 |
| RSAD2_IFI27_IFIT1_ISG15_RETN | 0.873 | 0.864 | 0.882 | 0.904 | 0.841 | 0.968 | 0.953 | 0.895 | 1 |
| RSAD2_IFI44L_SIGLEC1_SLC1A2_FAM20A | 0.873 | 0.863 | 0.883 | 0.903 | 0.840 | 0.965 | 0.943 | 0.871 | 1 |
| RSAD2_IFIT1_SIGLEC1_IL1R2_OLAH | 0.873 | 0.864 | 0.882 | 0.899 | 0.836 | 0.963 | 0.959 | 0.917 | 1 |
| OAS1_SIGLEC1_SLC1A2_FAM20A_MMP8 | 0.873 | 0.863 | 0.883 | 0.903 | 0.839 | 0.966 | 0.949 | 0.878 | 1 |
| RSAD2_ISG15_IL1R2_OLAH_FAM20A | 0.873 | 0.864 | 0.882 | 0.901 | 0.835 | 0.967 | 0.947 | 0.880 | 1 |
| IFIT1_IFI44L_ISG15_IL1R2_FAM20A | 0.873 | 0.864 | 0.882 | 0.900 | 0.836 | 0.963 | 0.948 | 0.883 | 1 |
| ISG15_HERC6_OLAH_FAM20A_MMP8 | 0.873 | 0.863 | 0.883 | 0.899 | 0.834 | 0.963 | 0.945 | 0.878 | 1 |
| IFIT1_SIGLEC1_IL1R2_RETN_MMP8 | 0.873 | 0.863 | 0.883 | 0.900 | 0.837 | 0.964 | 0.967 | 0.933 | 1 |
| IFI27_IFIT1_IFI44L_RETN_MMP8 | 0.873 | 0.862 | 0.883 | 0.904 | 0.841 | 0.968 | 0.950 | 0.891 | 1 |
| OAS1_IFI44L_SIGLEC1_HERC6_FAM20A | 0.873 | 0.863 | 0.882 | 0.898 | 0.832 | 0.964 | 0.954 | 0.892 | 1 |
| RSAD2_OAS1_IFI44L_SIGLEC1_IL1R2 | 0.873 | 0.864 | 0.882 | 0.895 | 0.829 | 0.960 | 0.961 | 0.922 | 1 |
| RSAD2_IFI27_OAS1_HERC6_MMP8 | 0.873 | 0.864 | 0.882 | 0.899 | 0.839 | 0.958 | 0.900 | 0.821 | 0.979 |
| IFI27_IFI44L_ISG15_SLC1A2_RETN | 0.873 | 0.863 | 0.882 | 0.905 | 0.842 | 0.968 | 0.945 | 0.89 | 1 |
| RSAD2_SIGLEC1_SLC1A2_FAM20A_RETN | 0.873 | 0.863 | 0.883 | 0.903 | 0.840 | 0.966 | 0.947 | 0.874 | 1 |
| IFI27_IFIT1_ISG15_RETN_MMP8 | 0.873 | 0.863 | 0.883 | 0.902 | 0.838 | 0.966 | 0.952 | 0.894 | 1 |
| IFI27_IFIT1_ISG15_HERC6_MMP8 | 0.873 | 0.864 | 0.882 | 0.897 | 0.837 | 0.957 | 0.895 | 0.813 | 0.976 |
| OAS1_IFI44L_ISG15_IL1R2_FAM20A | 0.873 | 0.863 | 0.882 | 0.900 | 0.836 | 0.965 | 0.943 | 0.869 | 1 |
| OAS1_SIGLEC1_ISG15_SLC1A2_FAM20A | 0.873 | 0.863 | 0.883 | 0.902 | 0.840 | 0.965 | 0.949 | 0.878 | 1 |
| IFI27_IFIT1_SIGLEC1_SLC1A2_MMP8 | 0.873 | 0.864 | 0.882 | 0.903 | 0.843 | 0.962 | 0.925 | 0.855 | 0.995 |
| OAS1_IFI44L_SIGLEC1_ISG15_IL1R2 | 0.873 | 0.864 | 0.881 | 0.900 | 0.836 | 0.963 | 0.957 | 0.915 | 0.998 |
| RSAD2_IFI44L_SLC1A2_IL1R2_FAM20A | 0.873 | 0.864 | 0.882 | 0.907 | 0.851 | 0.962 | 0.927 | 0.839 | 1 |
| OAS1_SIGLEC1_SLC1A2_FAM20A_RETN | 0.873 | 0.863 | 0.883 | 0.901 | 0.838 | 0.965 | 0.947 | 0.874 | 1 |
| OAS1_SIGLEC1_HERC6_SLC1A2_RETN | 0.873 | 0.863 | 0.882 | 0.904 | 0.843 | 0.965 | 0.951 | 0.901 | 1 |
| IFIT1_SIGLEC1_ISG15_IL1R2_MMP8 | 0.873 | 0.864 | 0.881 | 0.902 | 0.843 | 0.961 | 0.962 | 0.923 | 1 |
| IFIT1_SLC1A2_IL1R2_OLAH_FAM20A | 0.873 | 0.864 | 0.882 | 0.907 | 0.849 | 0.964 | 0.932 | 0.856 | 1 |
| OAS1_IL1R2_FAM20A_RETN_MMP8 | 0.873 | 0.863 | 0.882 | 0.904 | 0.842 | 0.967 | 0.937 | 0.858 | 1 |
| IFIT1_IFI44L_SIGLEC1_IL1R2_OLAH | 0.873 | 0.864 | 0.881 | 0.901 | 0.839 | 0.963 | 0.958 | 0.915 | 1 |
| RSAD2_IFI44L_SIGLEC1_ISG15_IL1R2 | 0.873 | 0.864 | 0.881 | 0.896 | 0.830 | 0.961 | 0.958 | 0.916 | 0.999 |
| RSAD2_IFI27_IFI44L_ISG15_RETN | 0.872 | 0.863 | 0.882 | 0.905 | 0.842 | 0.969 | 0.957 | 0.900 | 1 |
| OAS1_ISG15_SLC1A2_OLAH_FAM20A | 0.872 | 0.863 | 0.882 | 0.911 | 0.854 | 0.969 | 0.938 | 0.866 | 1 |
| IFI27_OAS1_IFIT1_SLC1A2_RETN | 0.872 | 0.863 | 0.882 | 0.901 | 0.837 | 0.966 | 0.942 | 0.870 | 1 |
| RSAD2_IFI27_OAS1_ISG15_RETN | 0.872 | 0.863 | 0.882 | 0.904 | 0.841 | 0.968 | 0.949 | 0.886 | 1 |
| OAS1_SIGLEC1_HERC6_FAM20A_MMP8 | 0.872 | 0.863 | 0.882 | 0.896 | 0.828 | 0.963 | 0.949 | 0.885 | 1 |
| RSAD2_SIGLEC1_ISG15_FAM20A_RETN | 0.872 | 0.863 | 0.882 | 0.895 | 0.825 | 0.966 | 0.953 | 0.895 | 1 |
| IFI27_IFIT1_ISG15_SLC1A2_RETN | 0.872 | 0.863 | 0.882 | 0.901 | 0.836 | 0.965 | 0.943 | 0.875 | 1 |
| IFI44L_ISG15_SLC1A2_OLAH_MMP8 | 0.872 | 0.863 | 0.881 | 0.906 | 0.848 | 0.964 | 0.937 | 0.882 | 0.991 |
| RSAD2_IFI27_IFIT1_IFI44L_RETN | 0.872 | 0.863 | 0.882 | 0.906 | 0.843 | 0.969 | 0.951 | 0.891 | 1 |
| OAS1_SIGLEC1_ISG15_IL1R2_OLAH | 0.872 | 0.863 | 0.881 | 0.900 | 0.837 | 0.963 | 0.955 | 0.912 | 0.999 |
| IFI44L_SLC1A2_IL1R2_OLAH_MMP8 | 0.872 | 0.863 | 0.881 | 0.904 | 0.847 | 0.960 | 0.932 | 0.870 | 0.993 |
| OAS1_SLC1A2_IL1R2_FAM20A_MMP8 | 0.872 | 0.864 | 0.881 | 0.911 | 0.858 | 0.964 | 0.926 | 0.835 | 1 |
| IFI44L_SLC1A2_IL1R2_OLAH_RETN | 0.872 | 0.863 | 0.882 | 0.909 | 0.852 | 0.966 | 0.930 | 0.871 | 0.990 |
| IFI27_OAS1_IFI44L_SLC1A2_RETN | 0.872 | 0.863 | 0.882 | 0.903 | 0.840 | 0.967 | 0.943 | 0.875 | 1 |
| IFIT1_SIGLEC1_HERC6_RETN_MMP8 | 0.872 | 0.862 | 0.882 | 0.893 | 0.823 | 0.963 | 0.925 | 0.865 | 0.985 |
| RSAD2_OAS1_SIGLEC1_ISG15_IL1R2 | 0.872 | 0.863 | 0.881 | 0.896 | 0.833 | 0.960 | 0.962 | 0.924 | 1 |
| IFI44L_ISG15_SLC1A2_IL1R2_OLAH | 0.872 | 0.863 | 0.881 | 0.906 | 0.850 | 0.963 | 0.936 | 0.879 | 0.993 |
| IFI27_OAS1_IFI44L_HERC6_MMP8 | 0.872 | 0.863 | 0.881 | 0.900 | 0.842 | 0.958 | 0.897 | 0.817 | 0.976 |
| RSAD2_SIGLEC1_ISG15_IL1R2_MMP8 | 0.872 | 0.863 | 0.881 | 0.903 | 0.844 | 0.962 | 0.965 | 0.929 | 1 |
| RSAD2_IFI44L_SIGLEC1_SLC1A2_IL1R2 | 0.872 | 0.863 | 0.881 | 0.909 | 0.853 | 0.965 | 0.966 | 0.931 | 1 |
| OAS1_HERC6_OLAH_FAM20A_RETN | 0.872 | 0.862 | 0.882 | 0.902 | 0.838 | 0.966 | 0.945 | 0.879 | 1 |
| RSAD2_OAS1_IL1R2_FAM20A_RETN | 0.872 | 0.863 | 0.881 | 0.898 | 0.832 | 0.964 | 0.934 | 0.854 | 1 |
| OAS1_SLC1A2_OLAH_FAM20A_RETN | 0.872 | 0.862 | 0.882 | 0.912 | 0.855 | 0.970 | 0.938 | 0.866 | 1 |
| RSAD2_HERC6_OLAH_FAM20A_RETN | 0.872 | 0.862 | 0.882 | 0.902 | 0.838 | 0.966 | 0.943 | 0.878 | 1 |
| IFI27_SLC1A2_FAM20A_RETN_MMP8 | 0.872 | 0.862 | 0.882 | 0.899 | 0.835 | 0.964 | 0.913 | 0.817 | 1 |
| RSAD2_OAS1_SLC1A2_IL1R2_FAM20A | 0.872 | 0.863 | 0.881 | 0.905 | 0.848 | 0.961 | 0.932 | 0.846 | 1 |
| RSAD2_IFI44L_HERC6_OLAH_FAM20A | 0.872 | 0.862 | 0.882 | 0.901 | 0.837 | 0.965 | 0.940 | 0.874 | 1 |
| OAS1_IFI44L_SIGLEC1_IL1R2_RETN | 0.872 | 0.863 | 0.881 | 0.897 | 0.832 | 0.963 | 0.954 | 0.911 | 0.998 |
| RSAD2_SIGLEC1_ISG15_FAM20A_MMP8 | 0.872 | 0.862 | 0.881 | 0.895 | 0.824 | 0.965 | 0.952 | 0.889 | 1 |
| OAS1_IFI44L_SIGLEC1_FAM20A_RETN | 0.872 | 0.862 | 0.882 | 0.894 | 0.822 | 0.966 | 0.954 | 0.895 | 1 |
| RSAD2_OAS1_SIGLEC1_IL1R2_RETN | 0.872 | 0.862 | 0.881 | 0.897 | 0.831 | 0.962 | 0.964 | 0.925 | 1 |
| RSAD2_IFIT1_SIGLEC1_HERC6_OLAH | 0.872 | 0.862 | 0.881 | 0.900 | 0.838 | 0.962 | 0.937 | 0.883 | 0.991 |
| IFIT1_IL1R2_FAM20A_RETN_MMP8 | 0.872 | 0.862 | 0.881 | 0.902 | 0.838 | 0.967 | 0.945 | 0.872 | 1 |
| OAS1_IFI44L_HERC6_OLAH_FAM20A | 0.872 | 0.862 | 0.881 | 0.902 | 0.838 | 0.966 | 0.947 | 0.885 | 1 |
| RSAD2_ISG15_HERC6_OLAH_FAM20A | 0.872 | 0.862 | 0.881 | 0.901 | 0.839 | 0.963 | 0.938 | 0.868 | 1 |
| IFI27_IFI44L_SLC1A2_RETN_MMP8 | 0.872 | 0.862 | 0.882 | 0.902 | 0.839 | 0.965 | 0.934 | 0.861 | 1 |
| IFI27_OAS1_IFI44L_RETN_MMP8 | 0.872 | 0.862 | 0.881 | 0.905 | 0.842 | 0.969 | 0.949 | 0.890 | 1 |
| SIGLEC1_ISG15_HERC6_FAM20A_MMP8 | 0.872 | 0.861 | 0.882 | 0.891 | 0.819 | 0.963 | 0.952 | 0.881 | 1 |
| OAS1_ISG15_SLC1A2_IL1R2_FAM20A | 0.872 | 0.862 | 0.881 | 0.905 | 0.848 | 0.962 | 0.927 | 0.841 | 1 |
| IFI44L_ISG15_IL1R2_FAM20A_RETN | 0.871 | 0.862 | 0.881 | 0.900 | 0.837 | 0.963 | 0.938 | 0.865 | 1 |
| OAS1_SIGLEC1_IL1R2_OLAH_RETN | 0.871 | 0.862 | 0.881 | 0.899 | 0.834 | 0.964 | 0.958 | 0.915 | 1 |
| OAS1_ISG15_IL1R2_FAM20A_RETN | 0.871 | 0.862 | 0.881 | 0.898 | 0.833 | 0.963 | 0.935 | 0.856 | 1 |
| OAS1_SIGLEC1_ISG15_IL1R2_MMP8 | 0.871 | 0.863 | 0.880 | 0.902 | 0.843 | 0.962 | 0.963 | 0.925 | 1 |
| IFIT1_IFI44L_SIGLEC1_IL1R2_RETN | 0.871 | 0.862 | 0.881 | 0.898 | 0.834 | 0.962 | 0.959 | 0.916 | 1 |
| RSAD2_IFIT1_SLC1A2_IL1R2_FAM20A | 0.871 | 0.863 | 0.880 | 0.904 | 0.845 | 0.962 | 0.935 | 0.854 | 1 |
| IFI27_OAS1_IFI44L_FAM20A_MMP8 | 0.871 | 0.861 | 0.882 | 0.899 | 0.832 | 0.966 | 0.937 | 0.858 | 1 |
| IFI27_ISG15_HERC6_SLC1A2_FAM20A | 0.871 | 0.861 | 0.882 | 0.905 | 0.846 | 0.964 | 0.933 | 0.850 | 1 |
| RSAD2_SLC1A2_OLAH_FAM20A_RETN | 0.871 | 0.862 | 0.881 | 0.908 | 0.849 | 0.967 | 0.932 | 0.856 | 1 |
| RSAD2_SIGLEC1_ISG15_IL1R2_OLAH | 0.871 | 0.862 | 0.880 | 0.899 | 0.836 | 0.962 | 0.960 | 0.919 | 1 |
| IFIT1_SIGLEC1_HERC6_SLC1A2_RETN | 0.871 | 0.862 | 0.880 | 0.904 | 0.844 | 0.963 | 0.929 | 0.871 | 0.987 |
| IFIT1_SIGLEC1_ISG15_IL1R2_RETN | 0.871 | 0.862 | 0.880 | 0.898 | 0.834 | 0.962 | 0.963 | 0.922 | 1 |
| RSAD2_ISG15_SLC1A2_OLAH_FAM20A | 0.871 | 0.862 | 0.881 | 0.908 | 0.849 | 0.967 | 0.932 | 0.858 | 1 |
| IFIT1_ISG15_SLC1A2_OLAH_FAM20A | 0.871 | 0.862 | 0.881 | 0.903 | 0.841 | 0.964 | 0.938 | 0.868 | 1 |
| OAS1_ISG15_HERC6_OLAH_FAM20A | 0.871 | 0.861 | 0.881 | 0.900 | 0.836 | 0.963 | 0.945 | 0.878 | 1 |
| SIGLEC1_ISG15_IL1R2_OLAH_RETN | 0.871 | 0.862 | 0.880 | 0.900 | 0.836 | 0.964 | 0.964 | 0.924 | 1 |
| IFI44L_ISG15_SLC1A2_OLAH_RETN | 0.871 | 0.862 | 0.881 | 0.908 | 0.850 | 0.967 | 0.933 | 0.876 | 0.990 |
| OAS1_IFIT1_SIGLEC1_OLAH_RETN | 0.871 | 0.862 | 0.881 | 0.895 | 0.825 | 0.964 | 0.942 | 0.889 | 0.995 |
| OAS1_IFI44L_SIGLEC1_SLC1A2_FAM20A | 0.871 | 0.861 | 0.881 | 0.903 | 0.841 | 0.966 | 0.948 | 0.878 | 1 |
| RSAD2_HERC6_OLAH_FAM20A_MMP8 | 0.871 | 0.862 | 0.881 | 0.898 | 0.834 | 0.962 | 0.941 | 0.873 | 1 |
| SIGLEC1_HERC6_OLAH_RETN_MMP8 | 0.871 | 0.861 | 0.882 | 0.892 | 0.820 | 0.965 | 0.961 | 0.922 | 1.000 |
| IFI27_OAS1_HERC6_SLC1A2_FAM20A | 0.871 | 0.861 | 0.881 | 0.904 | 0.846 | 0.963 | 0.933 | 0.850 | 1 |
| RSAD2_IFI27_HERC6_SLC1A2_FAM20A | 0.871 | 0.861 | 0.881 | 0.904 | 0.845 | 0.962 | 0.932 | 0.848 | 1 |
| IFIT1_IFI44L_SLC1A2_OLAH_MMP8 | 0.871 | 0.862 | 0.880 | 0.905 | 0.847 | 0.963 | 0.937 | 0.883 | 0.991 |
| OAS1_IFIT1_SIGLEC1_OLAH_MMP8 | 0.871 | 0.862 | 0.880 | 0.893 | 0.825 | 0.961 | 0.946 | 0.895 | 0.996 |
| RSAD2_ISG15_SLC1A2_IL1R2_FAM20A | 0.871 | 0.862 | 0.880 | 0.902 | 0.845 | 0.959 | 0.930 | 0.842 | 1 |
| RSAD2_SLC1A2_IL1R2_FAM20A_MMP8 | 0.871 | 0.863 | 0.879 | 0.906 | 0.851 | 0.960 | 0.926 | 0.832 | 1 |
| RSAD2_ISG15_HERC6_FAM20A_RETN | 0.871 | 0.861 | 0.881 | 0.900 | 0.837 | 0.963 | 0.938 | 0.862 | 1 |
| IFIT1_SIGLEC1_IL1R2_OLAH_RETN | 0.871 | 0.862 | 0.880 | 0.899 | 0.834 | 0.963 | 0.958 | 0.914 | 1 |
| IFIT1_SLC1A2_IL1R2_FAM20A_RETN | 0.871 | 0.861 | 0.880 | 0.906 | 0.849 | 0.963 | 0.934 | 0.855 | 1 |
| IFI27_IFI44L_HERC6_SLC1A2_FAM20A | 0.871 | 0.861 | 0.881 | 0.902 | 0.841 | 0.963 | 0.938 | 0.856 | 1 |
| RSAD2_IFI44L_SIGLEC1_IL1R2_RETN | 0.871 | 0.862 | 0.880 | 0.894 | 0.827 | 0.962 | 0.960 | 0.920 | 1.000 |
| RSAD2_IFI44L_SIGLEC1_ISG15_FAM20A | 0.871 | 0.861 | 0.881 | 0.894 | 0.823 | 0.965 | 0.951 | 0.892 | 1 |
| OAS1_HERC6_OLAH_FAM20A_MMP8 | 0.871 | 0.861 | 0.881 | 0.899 | 0.835 | 0.963 | 0.943 | 0.876 | 1 |
| IFI44L_SIGLEC1_HERC6_OLAH_RETN | 0.871 | 0.861 | 0.881 | 0.893 | 0.823 | 0.963 | 0.950 | 0.903 | 0.997 |
| RSAD2_IFIT1_SIGLEC1_ISG15_IL1R2 | 0.871 | 0.862 | 0.880 | 0.898 | 0.835 | 0.961 | 0.961 | 0.922 | 1 |
| IFI44L_SIGLEC1_HERC6_OLAH_MMP8 | 0.871 | 0.861 | 0.880 | 0.894 | 0.828 | 0.960 | 0.953 | 0.909 | 0.998 |
| RSAD2_IFI27_IFIT1_IFI44L_FAM20A | 0.871 | 0.861 | 0.881 | 0.900 | 0.831 | 0.968 | 0.941 | 0.865 | 1 |
| OAS1_IFI44L_SIGLEC1_ISG15_FAM20A | 0.871 | 0.861 | 0.881 | 0.893 | 0.821 | 0.965 | 0.957 | 0.896 | 1 |
| RSAD2_SIGLEC1_ISG15_HERC6_RETN | 0.871 | 0.861 | 0.881 | 0.896 | 0.828 | 0.965 | 0.939 | 0.886 | 0.992 |
| RSAD2_IFIT1_SLC1A2_OLAH_FAM20A | 0.871 | 0.861 | 0.880 | 0.906 | 0.846 | 0.966 | 0.938 | 0.866 | 1 |
| RSAD2_IFI27_ISG15_HERC6_MMP8 | 0.871 | 0.862 | 0.879 | 0.896 | 0.838 | 0.955 | 0.896 | 0.817 | 0.975 |
| RSAD2_IFIT1_ISG15_IL1R2_FAM20A | 0.871 | 0.861 | 0.880 | 0.900 | 0.835 | 0.965 | 0.950 | 0.883 | 1 |
| IFI27_OAS1_HERC6_SLC1A2_MMP8 | 0.871 | 0.862 | 0.880 | 0.904 | 0.849 | 0.959 | 0.897 | 0.813 | 0.980 |
| RSAD2_IFI27_SIGLEC1_SLC1A2_MMP8 | 0.871 | 0.861 | 0.880 | 0.901 | 0.841 | 0.961 | 0.932 | 0.865 | 0.998 |
| IFIT1_IFI44L_IL1R2_OLAH_FAM20A | 0.871 | 0.861 | 0.880 | 0.898 | 0.833 | 0.963 | 0.955 | 0.901 | 1 |
| RSAD2_IFIT1_HERC6_OLAH_FAM20A | 0.871 | 0.861 | 0.880 | 0.900 | 0.836 | 0.964 | 0.942 | 0.875 | 1 |
| IFI44L_SLC1A2_OLAH_RETN_MMP8 | 0.871 | 0.861 | 0.880 | 0.906 | 0.846 | 0.967 | 0.943 | 0.893 | 0.994 |
| OAS1_SIGLEC1_HERC6_OLAH_RETN | 0.871 | 0.861 | 0.880 | 0.894 | 0.826 | 0.961 | 0.954 | 0.910 | 0.999 |
| IFI27_OAS1_SIGLEC1_HERC6_SLC1A2 | 0.870 | 0.862 | 0.879 | 0.900 | 0.844 | 0.956 | 0.953 | 0.905 | 1 |
| IFI27_IFI44L_SIGLEC1_SLC1A2_MMP8 | 0.870 | 0.862 | 0.879 | 0.900 | 0.840 | 0.961 | 0.929 | 0.860 | 0.999 |
| IFI27_SIGLEC1_ISG15_SLC1A2_MMP8 | 0.870 | 0.861 | 0.880 | 0.903 | 0.844 | 0.961 | 0.930 | 0.863 | 0.997 |
| RSAD2_IFIT1_SIGLEC1_IL1R2_RETN | 0.870 | 0.861 | 0.880 | 0.897 | 0.832 | 0.963 | 0.960 | 0.919 | 1 |
| OAS1_IFI44L_IL1R2_FAM20A_RETN | 0.870 | 0.861 | 0.880 | 0.900 | 0.835 | 0.964 | 0.938 | 0.862 | 1 |
| RSAD2_IFI27_IFI44L_SLC1A2_FAM20A | 0.870 | 0.860 | 0.881 | 0.902 | 0.837 | 0.966 | 0.940 | 0.861 | 1 |
| IFI27_IFIT1_IFI44L_ISG15_FAM20A | 0.870 | 0.860 | 0.880 | 0.895 | 0.826 | 0.964 | 0.940 | 0.863 | 1 |
| IFI27_IFIT1_IFI44L_HERC6_MMP8 | 0.870 | 0.861 | 0.880 | 0.896 | 0.837 | 0.956 | 0.890 | 0.807 | 0.973 |
| RSAD2_IFI27_IFI44L_SLC1A2_RETN | 0.870 | 0.861 | 0.880 | 0.902 | 0.838 | 0.966 | 0.946 | 0.880 | 1 |
| OAS1_IFI44L_SIGLEC1_FAM20A_MMP8 | 0.870 | 0.860 | 0.881 | 0.892 | 0.820 | 0.964 | 0.955 | 0.893 | 1 |
| IFI27_OAS1_ISG15_SLC1A2_RETN | 0.870 | 0.861 | 0.880 | 0.901 | 0.837 | 0.965 | 0.940 | 0.870 | 1 |
| IFI44L_ISG15_HERC6_FAM20A_RETN | 0.870 | 0.860 | 0.880 | 0.896 | 0.827 | 0.965 | 0.945 | 0.870 | 1 |
| RSAD2_IFI27_OAS1_RETN_MMP8 | 0.870 | 0.860 | 0.880 | 0.901 | 0.836 | 0.966 | 0.943 | 0.880 | 1 |
| IFI27_IFI44L_ISG15_SLC1A2_FAM20A | 0.870 | 0.860 | 0.880 | 0.897 | 0.831 | 0.963 | 0.943 | 0.866 | 1 |
| IFIT1_ISG15_IL1R2_FAM20A_RETN | 0.870 | 0.860 | 0.880 | 0.901 | 0.838 | 0.964 | 0.949 | 0.884 | 1 |
| OAS1_ISG15_HERC6_IL1R2_RETN | 0.870 | 0.861 | 0.880 | 0.897 | 0.835 | 0.960 | 0.949 | 0.899 | 0.999 |
| RSAD2_OAS1_SIGLEC1_HERC6_RETN | 0.870 | 0.860 | 0.880 | 0.890 | 0.820 | 0.961 | 0.939 | 0.886 | 0.992 |
| IFIT1_IFI44L_HERC6_SLC1A2_OLAH | 0.870 | 0.860 | 0.880 | 0.905 | 0.848 | 0.963 | 0.940 | 0.886 | 0.994 |
| IFI44L_IL1R2_FAM20A_RETN_MMP8 | 0.870 | 0.861 | 0.879 | 0.899 | 0.836 | 0.962 | 0.938 | 0.860 | 1 |
| IFI27_OAS1_ISG15_RETN_MMP8 | 0.870 | 0.860 | 0.880 | 0.903 | 0.840 | 0.966 | 0.948 | 0.888 | 1 |
| RSAD2_IFI44L_IL1R2_OLAH_FAM20A | 0.870 | 0.860 | 0.880 | 0.898 | 0.832 | 0.964 | 0.950 | 0.888 | 1 |
| RSAD2_IFI44L_SIGLEC1_FAM20A_MMP8 | 0.870 | 0.860 | 0.880 | 0.894 | 0.824 | 0.964 | 0.947 | 0.881 | 1 |
| ISG15_IL1R2_OLAH_FAM20A_MMP8 | 0.870 | 0.861 | 0.879 | 0.900 | 0.840 | 0.960 | 0.930 | 0.855 | 1 |
| IFIT1_SLC1A2_OLAH_FAM20A_RETN | 0.870 | 0.860 | 0.880 | 0.903 | 0.841 | 0.964 | 0.937 | 0.866 | 1 |
| RSAD2_IFIT1_IL1R2_OLAH_FAM20A | 0.870 | 0.861 | 0.879 | 0.899 | 0.831 | 0.966 | 0.952 | 0.890 | 1 |
| IFI27_IFIT1_ISG15_FAM20A_MMP8 | 0.870 | 0.860 | 0.880 | 0.896 | 0.829 | 0.963 | 0.935 | 0.854 | 1 |
| RSAD2_IFI27_OAS1_SLC1A2_RETN | 0.870 | 0.860 | 0.880 | 0.901 | 0.836 | 0.966 | 0.938 | 0.863 | 1 |
| IFI44L_ISG15_HERC6_SLC1A2_OLAH | 0.870 | 0.860 | 0.880 | 0.904 | 0.846 | 0.962 | 0.939 | 0.885 | 0.994 |
| RSAD2_HERC6_SLC1A2_IL1R2_OLAH | 0.870 | 0.861 | 0.879 | 0.905 | 0.847 | 0.962 | 0.942 | 0.887 | 0.997 |
| RSAD2_IFI27_IFI44L_HERC6_MMP8 | 0.870 | 0.861 | 0.879 | 0.897 | 0.837 | 0.956 | 0.892 | 0.811 | 0.974 |
| RSAD2_SIGLEC1_IL1R2_OLAH_RETN | 0.870 | 0.860 | 0.879 | 0.898 | 0.832 | 0.963 | 0.961 | 0.919 | 1 |
| RSAD2_ISG15_IL1R2_FAM20A_MMP8 | 0.870 | 0.861 | 0.879 | 0.900 | 0.837 | 0.962 | 0.935 | 0.852 | 1 |
| OAS1_IFIT1_IFI44L_SIGLEC1_OLAH | 0.870 | 0.861 | 0.879 | 0.893 | 0.825 | 0.962 | 0.942 | 0.889 | 0.996 |
| IFI27_IFIT1_HERC6_SLC1A2_FAM20A | 0.870 | 0.860 | 0.880 | 0.903 | 0.844 | 0.962 | 0.933 | 0.850 | 1 |
| RSAD2_IFI27_IFI44L_RETN_MMP8 | 0.870 | 0.860 | 0.880 | 0.903 | 0.839 | 0.967 | 0.951 | 0.895 | 1 |
| OAS1_ISG15_HERC6_FAM20A_RETN | 0.870 | 0.860 | 0.880 | 0.898 | 0.833 | 0.963 | 0.938 | 0.862 | 1 |
| IFI44L_HERC6_SLC1A2_OLAH_MMP8 | 0.870 | 0.860 | 0.880 | 0.904 | 0.846 | 0.962 | 0.939 | 0.885 | 0.994 |
| IFI44L_HERC6_SLC1A2_IL1R2_OLAH | 0.870 | 0.860 | 0.880 | 0.907 | 0.851 | 0.963 | 0.942 | 0.888 | 0.997 |
| OAS1_SLC1A2_IL1R2_FAM20A_RETN | 0.870 | 0.861 | 0.879 | 0.906 | 0.849 | 0.962 | 0.926 | 0.839 | 1 |
| IFI44L_IL1R2_OLAH_FAM20A_RETN | 0.870 | 0.860 | 0.879 | 0.898 | 0.834 | 0.962 | 0.945 | 0.882 | 1 |
| OAS1_SIGLEC1_ISG15_FAM20A_MMP8 | 0.870 | 0.859 | 0.880 | 0.892 | 0.820 | 0.964 | 0.955 | 0.893 | 1 |
| IFIT1_SLC1A2_IL1R2_OLAH_MMP8 | 0.870 | 0.861 | 0.878 | 0.902 | 0.845 | 0.960 | 0.949 | 0.902 | 0.996 |
| IFI27_IFIT1_IFI44L_SLC1A2_RETN | 0.870 | 0.860 | 0.879 | 0.901 | 0.836 | 0.965 | 0.945 | 0.878 | 1 |
| IFI27_IFIT1_SLC1A2_RETN_MMP8 | 0.870 | 0.860 | 0.880 | 0.904 | 0.841 | 0.966 | 0.943 | 0.877 | 1 |
| RSAD2_IFI27_IFIT1_RETN_MMP8 | 0.869 | 0.860 | 0.879 | 0.905 | 0.842 | 0.968 | 0.950 | 0.889 | 1 |
| RSAD2_OAS1_IFIT1_SIGLEC1_OLAH | 0.869 | 0.860 | 0.879 | 0.894 | 0.825 | 0.962 | 0.946 | 0.894 | 0.997 |
| RSAD2_SIGLEC1_HERC6_SLC1A2_RETN | 0.869 | 0.860 | 0.879 | 0.902 | 0.840 | 0.964 | 0.940 | 0.887 | 0.993 |
| RETN_RSAD2_IFI44L_IL1R2_FAM20A_MMP8 | 0.869 | 0.860 | 0.879 | 0.900 | 0.837 | 0.962 | 0.936 | 0.855 | 1 |
| RSAD2_OAS1_SIGLEC1_HERC6_OLAH | 0.869 | 0.860 | 0.879 | 0.896 | 0.833 | 0.959 | 0.949 | 0.902 | 0.996 |
| OAS1_IFIT1_HERC6_IL1R2_MMP8 | 0.869 | 0.860 | 0.878 | 0.896 | 0.834 | 0.958 | 0.949 | 0.902 | 0.996 |
| RSAD2_IL1R2_OLAH_FAM20A_RETN | 0.869 | 0.860 | 0.879 | 0.897 | 0.830 | 0.963 | 0.945 | 0.874 | 1 |
| RSAD2_IFI44L_ISG15_IL1R2_FAM20A | 0.869 | 0.860 | 0.879 | 0.898 | 0.833 | 0.963 | 0.943 | 0.873 | 1 |
| OAS1_SIGLEC1_ISG15_IL1R2_RETN | 0.869 | 0.860 | 0.879 | 0.898 | 0.833 | 0.963 | 0.961 | 0.920 | 1 |
| RSAD2_IFI27_IFIT1_SLC1A2_RETN | 0.869 | 0.860 | 0.879 | 0.900 | 0.835 | 0.965 | 0.943 | 0.873 | 1 |
| RSAD2_SIGLEC1_ISG15_IL1R2_RETN | 0.869 | 0.860 | 0.879 | 0.897 | 0.832 | 0.963 | 0.963 | 0.923 | 1 |
| OAS1_IFI44L_SIGLEC1_HERC6_OLAH | 0.869 | 0.860 | 0.879 | 0.895 | 0.831 | 0.958 | 0.951 | 0.905 | 0.997 |
| IFI27_IFI44L_ISG15_HERC6_MMP8 | 0.869 | 0.860 | 0.878 | 0.894 | 0.832 | 0.956 | 0.895 | 0.814 | 0.975 |
| IFI44L_SIGLEC1_ISG15_HERC6_OLAH | 0.869 | 0.860 | 0.879 | 0.894 | 0.828 | 0.960 | 0.951 | 0.905 | 0.997 |
| RSAD2_IFI44L_SIGLEC1_FAM20A_RETN | 0.869 | 0.859 | 0.879 | 0.894 | 0.823 | 0.965 | 0.951 | 0.892 | 1 |
| RSAD2_SIGLEC1_HERC6_OLAH_RETN | 0.869 | 0.860 | 0.879 | 0.895 | 0.828 | 0.963 | 0.948 | 0.900 | 0.996 |
| ISG15_SLC1A2_IL1R2_FAM20A_RETN | 0.869 | 0.860 | 0.879 | 0.906 | 0.850 | 0.963 | 0.916 | 0.826 | 1 |
| IFI27_IFIT1_IFI44L_SLC1A2_FAM20A | 0.869 | 0.859 | 0.879 | 0.899 | 0.834 | 0.965 | 0.942 | 0.865 | 1 |
| IFIT1_IFI44L_HERC6_FAM20A_RETN | 0.869 | 0.859 | 0.879 | 0.896 | 0.827 | 0.964 | 0.928 | 0.848 | 1 |
| OAS1_IFIT1_ISG15_HERC6_IL1R2 | 0.869 | 0.860 | 0.878 | 0.897 | 0.839 | 0.956 | 0.947 | 0.897 | 0.997 |
| RSAD2_SLC1A2_IL1R2_FAM20A_RETN | 0.869 | 0.860 | 0.878 | 0.901 | 0.843 | 0.958 | 0.929 | 0.840 | 1 |
| IFIT1_IFI44L_ISG15_SLC1A2_OLAH | 0.869 | 0.860 | 0.878 | 0.907 | 0.849 | 0.964 | 0.934 | 0.878 | 0.990 |
| OAS1_IFIT1_HERC6_IL1R2_RETN | 0.869 | 0.859 | 0.879 | 0.893 | 0.827 | 0.960 | 0.949 | 0.902 | 0.996 |
| OAS1_HERC6_SLC1A2_IL1R2_OLAH | 0.869 | 0.860 | 0.878 | 0.904 | 0.847 | 0.961 | 0.939 | 0.883 | 0.995 |
| OAS1_IFIT1_SLC1A2_IL1R2_OLAH | 0.869 | 0.859 | 0.878 | 0.907 | 0.849 | 0.965 | 0.943 | 0.892 | 0.995 |
| RSAD2_SIGLEC1_HERC6_OLAH_MMP8 | 0.869 | 0.859 | 0.878 | 0.897 | 0.834 | 0.960 | 0.949 | 0.902 | 0.996 |
| OAS1_IFIT1_SIGLEC1_ISG15_OLAH | 0.869 | 0.860 | 0.878 | 0.894 | 0.826 | 0.962 | 0.941 | 0.888 | 0.995 |
| RSAD2_OAS1_IFIT1_HERC6_IL1R2 | 0.869 | 0.860 | 0.878 | 0.895 | 0.833 | 0.957 | 0.942 | 0.891 | 0.994 |
| RSAD2_OAS1_OLAH_FAM20A_MMP8 | 0.869 | 0.859 | 0.879 | 0.893 | 0.823 | 0.963 | 0.946 | 0.881 | 1 |
| IFIT1_IFI44L_SLC1A2_OLAH_RETN | 0.869 | 0.859 | 0.878 | 0.908 | 0.850 | 0.967 | 0.933 | 0.875 | 0.990 |
| RSAD2_IFI27_ISG15_SLC1A2_RETN | 0.869 | 0.859 | 0.878 | 0.901 | 0.837 | 0.965 | 0.939 | 0.868 | 1 |
| OAS1_IFI44L_SIGLEC1_HERC6_RETN | 0.869 | 0.859 | 0.878 | 0.889 | 0.817 | 0.961 | 0.946 | 0.896 | 0.995 |
| IFIT1_HERC6_IL1R2_RETN_MMP8 | 0.869 | 0.859 | 0.878 | 0.894 | 0.826 | 0.962 | 0.959 | 0.918 | 1.000 |
| RSAD2_IFI44L_HERC6_SLC1A2_OLAH | 0.869 | 0.859 | 0.878 | 0.905 | 0.849 | 0.962 | 0.934 | 0.876 | 0.991 |
| RSAD2_IFI27_ISG15_RETN_MMP8 | 0.869 | 0.859 | 0.878 | 0.901 | 0.837 | 0.965 | 0.951 | 0.894 | 1 |
| ISG15_HERC6_IL1R2_RETN_MMP8 | 0.869 | 0.859 | 0.878 | 0.897 | 0.833 | 0.960 | 0.948 | 0.895 | 1 |
| RSAD2_IFIT1_ISG15_HERC6_IL1R2 | 0.869 | 0.860 | 0.877 | 0.898 | 0.838 | 0.957 | 0.946 | 0.895 | 0.996 |
| ISG15_SLC1A2_IL1R2_FAM20A_MMP8 | 0.869 | 0.860 | 0.877 | 0.906 | 0.852 | 0.961 | 0.914 | 0.822 | 1 |
| IFIT1_HERC6_SLC1A2_IL1R2_OLAH | 0.869 | 0.859 | 0.878 | 0.904 | 0.846 | 0.961 | 0.942 | 0.889 | 0.996 |
| OAS1_IFI44L_HERC6_SLC1A2_OLAH | 0.869 | 0.858 | 0.879 | 0.905 | 0.848 | 0.962 | 0.938 | 0.883 | 0.994 |
| OAS1_IFIT1_HERC6_SLC1A2_IL1R2 | 0.869 | 0.859 | 0.878 | 0.902 | 0.845 | 0.959 | 0.946 | 0.893 | 0.998 |
| OAS1_SIGLEC1_HERC6_OLAH_MMP8 | 0.869 | 0.859 | 0.878 | 0.893 | 0.828 | 0.957 | 0.953 | 0.909 | 0.998 |
| IFIT1_ISG15_SLC1A2_IL1R2_OLAH | 0.869 | 0.859 | 0.878 | 0.907 | 0.849 | 0.965 | 0.948 | 0.899 | 0.996 |
| IFIT1_HERC6_SLC1A2_OLAH_MMP8 | 0.868 | 0.859 | 0.878 | 0.902 | 0.844 | 0.960 | 0.943 | 0.891 | 0.996 |
| IFI27_OAS1_IFI44L_ISG15_FAM20A | 0.868 | 0.858 | 0.879 | 0.893 | 0.824 | 0.963 | 0.942 | 0.863 | 1 |
| IFI44L_HERC6_SLC1A2_OLAH_RETN | 0.868 | 0.858 | 0.879 | 0.906 | 0.848 | 0.965 | 0.942 | 0.889 | 0.995 |
| RSAD2_OAS1_HERC6_SLC1A2_OLAH | 0.868 | 0.859 | 0.878 | 0.902 | 0.844 | 0.960 | 0.945 | 0.892 | 0.998 |
| IFI44L_ISG15_HERC6_IL1R2_RETN | 0.868 | 0.859 | 0.878 | 0.896 | 0.832 | 0.960 | 0.949 | 0.900 | 0.998 |
| SIGLEC1_ISG15_HERC6_OLAH_RETN | 0.868 | 0.858 | 0.879 | 0.892 | 0.820 | 0.964 | 0.957 | 0.914 | 0.999 |
| OAS1_IFIT1_SLC1A2_OLAH_MMP8 | 0.868 | 0.859 | 0.878 | 0.905 | 0.847 | 0.962 | 0.945 | 0.895 | 0.994 |
| IFIT1_SIGLEC1_OLAH_RETN_MMP8 | 0.868 | 0.858 | 0.878 | 0.894 | 0.826 | 0.962 | 0.959 | 0.915 | 1 |
| IFI27_ISG15_SLC1A2_RETN_MMP8 | 0.868 | 0.858 | 0.878 | 0.900 | 0.838 | 0.962 | 0.929 | 0.851 | 1 |
| IFIT1_ISG15_HERC6_IL1R2_MMP8 | 0.868 | 0.859 | 0.877 | 0.896 | 0.836 | 0.957 | 0.948 | 0.899 | 0.996 |
| IFI27_OAS1_IFIT1_SIGLEC1_MMP8 | 0.868 | 0.859 | 0.878 | 0.888 | 0.821 | 0.956 | 0.913 | 0.837 | 0.989 |
| IFIT1_IFI44L_SLC1A2_IL1R2_OLAH | 0.868 | 0.859 | 0.878 | 0.910 | 0.854 | 0.966 | 0.946 | 0.896 | 0.996 |
| IFI27_OAS1_IFIT1_FAM20A_MMP8 | 0.868 | 0.858 | 0.878 | 0.894 | 0.826 | 0.962 | 0.937 | 0.857 | 1 |
| IFIT1_ISG15_HERC6_IL1R2_RETN | 0.868 | 0.859 | 0.878 | 0.896 | 0.832 | 0.960 | 0.948 | 0.898 | 0.997 |
| IFI27_IFIT1_SIGLEC1_ISG15_MMP8 | 0.868 | 0.859 | 0.878 | 0.888 | 0.821 | 0.956 | 0.911 | 0.835 | 0.987 |
| IFI44L_SIGLEC1_OLAH_RETN_MMP8 | 0.868 | 0.858 | 0.878 | 0.891 | 0.821 | 0.961 | 0.962 | 0.921 | 1 |
| IFI27_OAS1_ISG15_FAM20A_MMP8 | 0.868 | 0.858 | 0.878 | 0.893 | 0.824 | 0.961 | 0.936 | 0.856 | 1 |
| ISG15_HERC6_SLC1A2_IL1R2_OLAH | 0.868 | 0.858 | 0.877 | 0.904 | 0.848 | 0.961 | 0.936 | 0.878 | 0.994 |
| IFIT1_HERC6_SLC1A2_IL1R2_MMP8 | 0.868 | 0.859 | 0.877 | 0.903 | 0.847 | 0.960 | 0.951 | 0.903 | 1.000 |
| IFIT1_IFI44L_ISG15_HERC6_IL1R2 | 0.868 | 0.859 | 0.877 | 0.896 | 0.836 | 0.957 | 0.946 | 0.895 | 0.996 |
| IFI27_IFI44L_SIGLEC1_ISG15_MMP8 | 0.868 | 0.859 | 0.877 | 0.889 | 0.823 | 0.956 | 0.916 | 0.844 | 0.988 |
| RSAD2_IFI27_OAS1_FAM20A_MMP8 | 0.868 | 0.858 | 0.878 | 0.893 | 0.824 | 0.961 | 0.937 | 0.857 | 1 |
| OAS1_IFIT1_HERC6_SLC1A2_OLAH | 0.868 | 0.858 | 0.878 | 0.902 | 0.844 | 0.960 | 0.945 | 0.893 | 0.996 |
| IFI27_SIGLEC1_HERC6_SLC1A2_MMP8 | 0.868 | 0.858 | 0.877 | 0.902 | 0.844 | 0.960 | 0.927 | 0.859 | 0.995 |
| RSAD2_ISG15_HERC6_IL1R2_RETN | 0.868 | 0.859 | 0.877 | 0.897 | 0.834 | 0.960 | 0.948 | 0.898 | 0.997 |
| IFI44L_ISG15_HERC6_IL1R2_MMP8 | 0.868 | 0.859 | 0.877 | 0.895 | 0.836 | 0.954 | 0.943 | 0.893 | 0.994 |
| IFIT1_IFI44L_HERC6_IL1R2_MMP8 | 0.868 | 0.859 | 0.877 | 0.896 | 0.833 | 0.958 | 0.950 | 0.903 | 0.997 |
| OAS1_HERC6_SLC1A2_OLAH_MMP8 | 0.868 | 0.858 | 0.877 | 0.902 | 0.844 | 0.960 | 0.935 | 0.877 | 0.993 |
| OAS1_SIGLEC1_HERC6_RETN_MMP8 | 0.868 | 0.858 | 0.878 | 0.892 | 0.820 | 0.963 | 0.947 | 0.897 | 0.997 |
| IFI27_IFIT1_IFI44L_SIGLEC1_MMP8 | 0.868 | 0.858 | 0.877 | 0.891 | 0.825 | 0.957 | 0.913 | 0.838 | 0.988 |
| RSAD2_OAS1_SIGLEC1_OLAH_MMP8 | 0.868 | 0.858 | 0.877 | 0.892 | 0.825 | 0.960 | 0.948 | 0.899 | 0.997 |
| IFIT1_IFI44L_IL1R2_FAM20A_RETN | 0.868 | 0.858 | 0.877 | 0.896 | 0.831 | 0.960 | 0.947 | 0.881 | 1 |
| ISG15_HERC6_SLC1A2_OLAH_MMP8 | 0.868 | 0.858 | 0.877 | 0.903 | 0.845 | 0.960 | 0.940 | 0.886 | 0.995 |
| RSAD2_IFI27_IFIT1_HERC6_MMP8 | 0.868 | 0.859 | 0.877 | 0.897 | 0.838 | 0.956 | 0.892 | 0.812 | 0.973 |
| RSAD2_ISG15_IL1R2_FAM20A_RETN | 0.868 | 0.858 | 0.877 | 0.895 | 0.829 | 0.961 | 0.942 | 0.868 | 1 |
| ISG15_HERC6_SLC1A2_FAM20A_RETN | 0.868 | 0.858 | 0.878 | 0.899 | 0.835 | 0.963 | 0.940 | 0.860 | 1 |
| OAS1_IFIT1_IFI44L_SLC1A2_OLAH | 0.868 | 0.858 | 0.878 | 0.908 | 0.851 | 0.965 | 0.936 | 0.881 | 0.990 |
| IFIT1_ISG15_HERC6_SLC1A2_IL1R2 | 0.868 | 0.859 | 0.877 | 0.903 | 0.846 | 0.959 | 0.951 | 0.902 | 1 |
| IFI44L_ISG15_HERC6_IL1R2_OLAH | 0.868 | 0.859 | 0.877 | 0.899 | 0.839 | 0.959 | 0.947 | 0.898 | 0.996 |
| RSAD2_HERC6_SLC1A2_OLAH_MMP8 | 0.868 | 0.858 | 0.877 | 0.901 | 0.843 | 0.960 | 0.939 | 0.883 | 0.995 |
| OAS1_IFIT1_IFI44L_HERC6_IL1R2 | 0.868 | 0.859 | 0.877 | 0.896 | 0.833 | 0.958 | 0.942 | 0.891 | 0.994 |
| SIGLEC1_ISG15_HERC6_OLAH_MMP8 | 0.868 | 0.858 | 0.877 | 0.891 | 0.823 | 0.959 | 0.954 | 0.911 | 0.998 |
| IFIT1_ISG15_HERC6_SLC1A2_OLAH | 0.868 | 0.858 | 0.877 | 0.903 | 0.844 | 0.961 | 0.943 | 0.891 | 0.996 |
| RSAD2_OAS1_HERC6_IL1R2_OLAH | 0.868 | 0.858 | 0.877 | 0.899 | 0.838 | 0.959 | 0.939 | 0.885 | 0.993 |
| IFIT1_ISG15_SLC1A2_OLAH_MMP8 | 0.868 | 0.858 | 0.877 | 0.904 | 0.845 | 0.962 | 0.946 | 0.897 | 0.995 |
| IFI27_OAS1_SLC1A2_RETN_MMP8 | 0.868 | 0.858 | 0.877 | 0.900 | 0.837 | 0.962 | 0.926 | 0.848 | 1 |
| IFIT1_IL1R2_OLAH_FAM20A_RETN | 0.868 | 0.858 | 0.877 | 0.895 | 0.831 | 0.960 | 0.951 | 0.891 | 1 |
| RSAD2_IFI27_SLC1A2_RETN_MMP8 | 0.868 | 0.858 | 0.877 | 0.901 | 0.839 | 0.964 | 0.932 | 0.858 | 1 |
| RSAD2_IFIT1_IFI44L_IL1R2_FAM20A | 0.868 | 0.859 | 0.877 | 0.895 | 0.829 | 0.961 | 0.949 | 0.881 | 1 |
| IFIT1_IFI44L_HERC6_IL1R2_RETN | 0.868 | 0.858 | 0.877 | 0.893 | 0.826 | 0.960 | 0.946 | 0.897 | 0.995 |
| OAS1_ISG15_HERC6_SLC1A2_OLAH | 0.868 | 0.858 | 0.877 | 0.902 | 0.844 | 0.959 | 0.941 | 0.888 | 0.995 |
| IFI27_OAS1_IFIT1_SIGLEC1_SLC1A2 | 0.867 | 0.858 | 0.877 | 0.897 | 0.837 | 0.956 | 0.953 | 0.903 | 1 |
| ISG15_HERC6_SLC1A2_OLAH_RETN | 0.867 | 0.858 | 0.877 | 0.903 | 0.844 | 0.963 | 0.943 | 0.888 | 0.999 |
| IFI27_SIGLEC1_ISG15_HERC6_MMP8 | 0.867 | 0.858 | 0.877 | 0.892 | 0.826 | 0.957 | 0.914 | 0.839 | 0.989 |
| RSAD2_SIGLEC1_ISG15_HERC6_OLAH | 0.867 | 0.858 | 0.877 | 0.897 | 0.834 | 0.960 | 0.948 | 0.900 | 0.995 |
| IFI27_IFIT1_HERC6_SLC1A2_MMP8 | 0.867 | 0.858 | 0.877 | 0.901 | 0.844 | 0.958 | 0.899 | 0.815 | 0.983 |
| OAS1_IFI44L_SLC1A2_OLAH_MMP8 | 0.867 | 0.858 | 0.877 | 0.905 | 0.847 | 0.962 | 0.938 | 0.884 | 0.992 |
| IFI27_OAS1_IFIT1_IFI44L_FAM20A | 0.867 | 0.857 | 0.878 | 0.894 | 0.825 | 0.964 | 0.941 | 0.862 | 1 |
| SIGLEC1_ISG15_OLAH_RETN_MMP8 | 0.867 | 0.857 | 0.877 | 0.891 | 0.820 | 0.961 | 0.964 | 0.925 | 1 |
| RSAD2_IFI44L_SLC1A2_OLAH_MMP8 | 0.867 | 0.858 | 0.877 | 0.905 | 0.847 | 0.963 | 0.937 | 0.882 | 0.991 |
| RSAD2_IFIT1_IL1R2_FAM20A_RETN | 0.867 | 0.858 | 0.877 | 0.897 | 0.831 | 0.963 | 0.946 | 0.874 | 1 |
| OAS1_SIGLEC1_ISG15_HERC6_OLAH | 0.867 | 0.858 | 0.877 | 0.892 | 0.827 | 0.956 | 0.954 | 0.911 | 0.998 |
| IFIT1_SLC1A2_IL1R2_OLAH_RETN | 0.867 | 0.858 | 0.877 | 0.908 | 0.850 | 0.965 | 0.943 | 0.892 | 0.995 |
| IFIT1_IFI44L_SIGLEC1_OLAH_MMP8 | 0.867 | 0.858 | 0.877 | 0.891 | 0.824 | 0.958 | 0.943 | 0.890 | 0.997 |
| RSAD2_ISG15_HERC6_SLC1A2_OLAH | 0.867 | 0.858 | 0.876 | 0.901 | 0.843 | 0.959 | 0.940 | 0.885 | 0.995 |
| RSAD2_IFIT1_SLC1A2_IL1R2_OLAH | 0.867 | 0.858 | 0.876 | 0.905 | 0.847 | 0.963 | 0.947 | 0.897 | 0.997 |
| RSAD2_OAS1_IFIT1_OLAH_FAM20A | 0.867 | 0.857 | 0.877 | 0.893 | 0.822 | 0.964 | 0.945 | 0.879 | 1 |
| RSAD2_IFI27_HERC6_SLC1A2_MMP8 | 0.867 | 0.858 | 0.876 | 0.900 | 0.842 | 0.957 | 0.900 | 0.817 | 0.983 |
| RSAD2_HERC6_SLC1A2_OLAH_RETN | 0.867 | 0.857 | 0.877 | 0.904 | 0.846 | 0.963 | 0.936 | 0.876 | 0.996 |
| ISG15_IL1R2_OLAH_FAM20A_RETN | 0.867 | 0.857 | 0.877 | 0.901 | 0.839 | 0.962 | 0.927 | 0.851 | 1 |
| IFI27_OAS1_IFI44L_SIGLEC1_MMP8 | 0.867 | 0.858 | 0.876 | 0.889 | 0.822 | 0.956 | 0.920 | 0.848 | 0.991 |
| OAS1_HERC6_SLC1A2_OLAH_RETN | 0.867 | 0.857 | 0.877 | 0.902 | 0.843 | 0.961 | 0.933 | 0.871 | 0.994 |
| RSAD2_OAS1_ISG15_OLAH_FAM20A | 0.867 | 0.857 | 0.877 | 0.892 | 0.821 | 0.964 | 0.949 | 0.885 | 1 |
| RSAD2_IFIT1_HERC6_SLC1A2_IL1R2 | 0.867 | 0.858 | 0.876 | 0.902 | 0.844 | 0.959 | 0.946 | 0.893 | 0.998 |
| IFI44L_HERC6_IL1R2_RETN_MMP8 | 0.867 | 0.857 | 0.877 | 0.895 | 0.829 | 0.961 | 0.950 | 0.903 | 0.997 |
| RSAD2_ISG15_HERC6_IL1R2_OLAH | 0.867 | 0.858 | 0.876 | 0.899 | 0.839 | 0.959 | 0.945 | 0.895 | 0.994 |
| ISG15_SLC1A2_OLAH_FAM20A_MMP8 | 0.867 | 0.857 | 0.877 | 0.901 | 0.843 | 0.960 | 0.921 | 0.842 | 0.999 |
| HERC6_SLC1A2_IL1R2_OLAH_RETN | 0.867 | 0.858 | 0.876 | 0.904 | 0.846 | 0.961 | 0.932 | 0.867 | 0.996 |
| RSAD2_OAS1_IFI44L_OLAH_FAM20A | 0.867 | 0.857 | 0.877 | 0.892 | 0.821 | 0.963 | 0.945 | 0.885 | 1 |
| RSAD2_IFI44L_SIGLEC1I_HERC6_OLAH | 0.867 | 0.857 | 0.876 | 0.896 | 0.833 | 0.960 | 0.949 | 0.902 | 0.996 |
| OAS1_HERC6_IL1R2_RETN_MMP8 | 0.867 | 0.857 | 0.876 | 0.893 | 0.827 | 0.959 | 0.941 | 0.887 | 0.996 |
| RSAD2_IFI27_IFIT1_FAM20A_MMP8 | 0.867 | 0.856 | 0.877 | 0.894 | 0.827 | 0.961 | 0.934 | 0.853 | 1 |
| IFI44L_SIGLEC1_ISG15_OLAH_MMP8 | 0.867 | 0.857 | 0.876 | 0.891 | 0.824 | 0.958 | 0.954 | 0.909 | 0.999 |
| OAS1_IFI44L_SLC1A2_OLAH_RETN | 0.867 | 0.857 | 0.877 | 0.909 | 0.851 | 0.966 | 0.936 | 0.881 | 0.991 |
| ISG15_HERC6_IL1R2_OLAH_MMP8 | 0.867 | 0.857 | 0.876 | 0.900 | 0.840 | 0.959 | 0.949 | 0.902 | 0.996 |
| RSAD2_IFIT1_HERC6_IL1R2_MMP8 | 0.867 | 0.858 | 0.876 | 0.896 | 0.834 | 0.958 | 0.949 | 0.902 | 0.996 |
| ISG15_HERC6_IL1R2_OLAH_RETN | 0.867 | 0.857 | 0.877 | 0.899 | 0.835 | 0.963 | 0.951 | 0.905 | 0.997 |
| RSAD2_IFIT1_SIGLEC1_OLAH_RETN | 0.867 | 0.857 | 0.876 | 0.895 | 0.826 | 0.964 | 0.949 | 0.897 | 1 |
| RSAD2_SLC1A2_IL1R2_OLAH_MMP8 | 0.867 | 0.858 | 0.876 | 0.903 | 0.846 | 0.960 | 0.939 | 0.880 | 0.998 |
| OAS1_ISG15_HERC6_IL1R2_MMP8 | 0.867 | 0.858 | 0.876 | 0.896 | 0.837 | 0.955 | 0.941 | 0.880 | 0.998 |
| OAS1_IFIT1_OLAH_FAM20A_MMP8 | 0.867 | 0.857 | 0.877 | 0.893 | 0.823 | 0.964 | 0.953 | 0.895 | 1 |
| OAS1_IFIT1_HERC6_IL1R2_OLAH | 0.867 | 0.857 | 0.876 | 0.899 | 0.837 | 0.960 | 0.946 | 0.896 | 0.995 |
| IFI44L_HERC6_IL1R2_OLAH_MMP8 | 0.867 | 0.857 | 0.876 | 0.899 | 0.840 | 0.958 | 0.951 | 0.904 | 0.998 |
| IFI27_OAS1_IFI44L_SLC1A2_FAM20A | 0.867 | 0.856 | 0.877 | 0.898 | 0.833 | 0.964 | 0.943 | 0.866 | 1 |
| RSAD2_IFIT1_HERC6_SLC1A2_OLAH | 0.867 | 0.857 | 0.876 | 0.902 | 0.844 | 0.960 | 0.940 | 0.885 | 0.995 |
| IFIT1_HERC6_IL1R2_OLAH_MMP8 | 0.867 | 0.857 | 0.876 | 0.899 | 0.839 | 0.959 | 0.955 | 0.912 | 0.999 |
| RSAD2_IFI27_ISG15_FAM20A_MMP8 | 0.867 | 0.856 | 0.877 | 0.894 | 0.826 | 0.962 | 0.934 | 0.853 | 1 |
| OAS1_IFIT1_OLAH_FAM20A_RETN | 0.867 | 0.857 | 0.876 | 0.893 | 0.822 | 0.965 | 0.949 | 0.886 | 1 |
| OAS1_SIGLEC1_OLAH_RETN_MMP8 | 0.867 | 0.856 | 0.877 | 0.892 | 0.821 | 0.962 | 0.964 | 0.925 | 1 |
| OAS1_HERC6_IL1R2_OLAH_MMP8 | 0.867 | 0.857 | 0.876 | 0.900 | 0.840 | 0.959 | 0.940 | 0.886 | 0.995 |
| OAS1_IFIT1_ISG15_OLAH_FAM20A | 0.867 | 0.857 | 0.876 | 0.891 | 0.819 | 0.963 | 0.951 | 0.891 | 1 |
| IFIT1_ISG15_HERC6_IL1R2_OLAH | 0.866 | 0.857 | 0.876 | 0.899 | 0.839 | 0.960 | 0.952 | 0.907 | 0.997 |
| RSAD2_OAS1_OLAH_FAM20A_RETN | 0.866 | 0.856 | 0.877 | 0.894 | 0.823 | 0.965 | 0.948 | 0.885 | 1 |
| ISG15_IL1R2_FAM20A_RETN_MMP8 | 0.866 | 0.858 | 0.875 | 0.901 | 0.842 | 0.960 | 0.921 | 0.836 | 1 |
| IFIT1_HERC6_SLC1A2_OLAH_RETN | 0.866 | 0.856 | 0.876 | 0.903 | 0.843 | 0.962 | 0.937 | 0.877 | 0.997 |
| IFI27_IFI44L_HERC6_SLC1A2_MMP8 | 0.866 | 0.857 | 0.876 | 0.899 | 0.842 | 0.957 | 0.899 | 0.815 | 0.983 |
| HERC6_IL1R2_OLAH_RETN_MMP8 | 0.866 | 0.856 | 0.877 | 0.899 | 0.836 | 0.962 | 0.953 | 0.909 | 0.997 |
| RSAD2_IL1R2_FAM20A_RETN_MMP8 | 0.866 | 0.857 | 0.876 | 0.899 | 0.835 | 0.963 | 0.937 | 0.856 | 1 |
| OAS1_IFIT1_ISG15_SLC1A2_OLAH | 0.866 | 0.857 | 0.876 | 0.905 | 0.847 | 0.962 | 0.941 | 0.889 | 0.993 |
| HERC6_SLC1A2_OLAH_RETN_MMP8 | 0.866 | 0.857 | 0.876 | 0.902 | 0.842 | 0.962 | 0.943 | 0.890 | 0.997 |
| OAS1_ISG15_HERC6_SLC1A2_IL1R2 | 0.866 | 0.857 | 0.876 | 0.902 | 0.846 | 0.957 | 0.933 | 0.867 | 0.998 |
| RSAD2_IFI27_SIGLEC1_HERC6_SLC1A2 | 0.866 | 0.857 | 0.875 | 0.897 | 0.841 | 0.953 | 0.945 | 0.892 | 0.997 |
| RSAD2_IFIT1_HERC6_IL1R2_RETN | 0.866 | 0.857 | 0.876 | 0.893 | 0.826 | 0.960 | 0.948 | 0.900 | 0.996 |
| IFIT1_ISG15_SLC1A2_OLAH_RETN | 0.866 | 0.857 | 0.876 | 0.907 | 0.848 | 0.965 | 0.951 | 0.905 | 0.997 |
| ISG15_HERC6_SLC1A2_IL1R2_RETN | 0.866 | 0.857 | 0.876 | 0.901 | 0.843 | 0.959 | 0.936 | 0.871 | 1 |
| RSAD2_ISG15_HERC6_IL1R2_MMP8 | 0.866 | 0.857 | 0.875 | 0.898 | 0.840 | 0.957 | 0.941 | 0.889 | 0.994 |
| IFI44L_HERC6_IL1R2_OLAH_RETN | 0.866 | 0.856 | 0.876 | 0.898 | 0.833 | 0.963 | 0.948 | 0.899 | 0.996 |
| OAS1_IFI44L_HERC6_IL1R2_RETN | 0.866 | 0.857 | 0.876 | 0.894 | 0.827 | 0.960 | 0.942 | 0.891 | 0.994 |
| RSAD2_OAS1_IFI44L_SLC1A2_OLAH | 0.866 | 0.856 | 0.876 | 0.907 | 0.850 | 0.964 | 0.934 | 0.878 | 0.990 |
| IFIT1_HERC6_SLC1A2_IL1R2_RETN | 0.866 | 0.856 | 0.876 | 0.901 | 0.842 | 0.960 | 0.940 | 0.883 | 0.998 |
| RSAD2_OAS1_HERC6_IL1R2_RETN | 0.866 | 0.857 | 0.876 | 0.894 | 0.827 | 0.960 | 0.942 | 0.891 | 0.994 |
| RSAD2_IFI44L_SLC1A2_OLAH_RETN | 0.866 | 0.856 | 0.876 | 0.909 | 0.851 | 0.967 | 0.936 | 0.881 | 0.991 |
| OAS1_IFIT1_SLC1A2_OLAH_RETN | 0.866 | 0.856 | 0.876 | 0.908 | 0.850 | 0.966 | 0.943 | 0.893 | 0.994 |
| IFI44L_HERC6_SLC1A2_IL1R2_RETN | 0.866 | 0.857 | 0.876 | 0.901 | 0.843 | 0.960 | 0.933 | 0.867 | 0.998 |
| RSAD2_IFI44L_SLC1A2_IL1R2_OLAH | 0.866 | 0.856 | 0.876 | 0.905 | 0.848 | 0.963 | 0.941 | 0.887 | 0.995 |
| IFI27_OAS1_SIGLEC1_ISG15_HERC6 | 0.866 | 0.856 | 0.876 | 0.895 | 0.839 | 0.951 | 0.936 | 0.881 | 0.991 |
| IFIT1_IFI44L_SIGLEC1_OLAH_RETN | 0.866 | 0.857 | 0.875 | 0.891 | 0.821 | 0.961 | 0.939 | 0.881 | 0.998 |
| RSAD2_OAS1_SLC1A2_IL1R2_OLAH | 0.866 | 0.857 | 0.875 | 0.904 | 0.847 | 0.962 | 0.938 | 0.880 | 0.996 |
| IFIT1_SIGLEC1_ISG15_OLAH_MMP8 | 0.866 | 0.857 | 0.875 | 0.891 | 0.825 | 0.958 | 0.950 | 0.902 | 0.998 |
| RSAD2_IFIT1_IFI44L_HERC6_IL1R2 | 0.866 | 0.857 | 0.875 | 0.896 | 0.833 | 0.958 | 0.943 | 0.893 | 0.994 |
| HERC6_SLC1A2_IL1R2_OLAH_MMP8 | 0.866 | 0.857 | 0.875 | 0.901 | 0.844 | 0.958 | 0.935 | 0.874 | 0.995 |
| RSAD2_IFIT1_SIGLEC1_OLAH_MMP8 | 0.866 | 0.857 | 0.875 | 0.893 | 0.826 | 0.961 | 0.951 | 0.903 | 0.999 |
| RSAD2_IFIT1_SLC1A2_OLAH_MMP8 | 0.866 | 0.857 | 0.875 | 0.904 | 0.846 | 0.962 | 0.945 | 0.895 | 0.994 |
| IFI27_IFIT1_SLC1A2_FAM20A_MMP8 | 0.866 | 0.855 | 0.876 | 0.897 | 0.832 | 0.961 | 0.933 | 0.851 | 1 |
| RSAD2_IFI44L_SIGLEC1_HERC6_RETN | 0.866 | 0.856 | 0.875 | 0.890 | 0.819 | 0.962 | 0.935 | 0.880 | 0.989 |
| IFIT1_IFI44L_SIGLEC1_ISG15_OLAH | 0.866 | 0.857 | 0.875 | 0.894 | 0.828 | 0.961 | 0.941 | 0.886 | 0.996 |
| RSAD2_OAS1_HERC6_IL1R2_MMP8 | 0.866 | 0.857 | 0.875 | 0.895 | 0.833 | 0.956 | 0.942 | 0.891 | 0.994 |
| OAS1_IFIT1_SLC1A2_IL1R2_MMP8 | 0.866 | 0.857 | 0.875 | 0.904 | 0.847 | 0.961 | 0.951 | 0.904 | 0.998 |
| ISG15_HERC6_FAM20A_RETN_MMP8 | 0.866 | 0.855 | 0.876 | 0.895 | 0.826 | 0.963 | 0.942 | 0.869 | 1 |
| RSAD2_OAS1_IFIT1_SLC1A2_OLAH | 0.866 | 0.856 | 0.876 | 0.904 | 0.846 | 0.962 | 0.941 | 0.889 | 0.993 |
| RSAD2_OAS1_ISG15_HERC6_IL1R2 | 0.866 | 0.857 | 0.875 | 0.897 | 0.839 | 0.955 | 0.943 | 0.891 | 0.996 |
| OAS1_ISG15_HERC6_OLAH_RETN | 0.866 | 0.856 | 0.876 | 0.895 | 0.830 | 0.960 | 0.954 | 0.910 | 0.999 |
| RSAD2_IFIT1_IFI44L_SLC1A2_OLAH | 0.866 | 0.856 | 0.875 | 0.907 | 0.850 | 0.965 | 0.935 | 0.880 | 0.990 |
| OAS1_IFI44L_SLC1A2_IL1R2_OLAH | 0.866 | 0.856 | 0.876 | 0.907 | 0.850 | 0.964 | 0.939 | 0.883 | 0.995 |
| RSAD2_ISG15_HERC6_SLC1A2_IL1R2 | 0.866 | 0.857 | 0.875 | 0.899 | 0.843 | 0.956 | 0.937 | 0.874 | 1 |
| OAS1_IFI44L_ISG15_SLC1A2_OLAH | 0.866 | 0.856 | 0.876 | 0.907 | 0.850 | 0.964 | 0.934 | 0.878 | 0.990 |
| IFIT1_IFI44L_HERC6_IL1R2_OLAH | 0.866 | 0.857 | 0.875 | 0.899 | 0.838 | 0.960 | 0.946 | 0.896 | 0.995 |
| RSAD2_HERC6_IL1R2_OLAH_MMP8 | 0.866 | 0.856 | 0.875 | 0.898 | 0.839 | 0.958 | 0.947 | 0.897 | 0.996 |
| RSAD2_SLC1A2_IL1R2_OLAH_RETN | 0.866 | 0.856 | 0.875 | 0.905 | 0.847 | 0.962 | 0.937 | 0.877 | 0.997 |
| RSAD2_OAS1_SIGLEC1_OLAH_RETN | 0.866 | 0.856 | 0.876 | 0.890 | 0.820 | 0.961 | 0.948 | 0.898 | 0.998 |
| OAS1_IFI44L_HERC6_IL1R2_OLAH | 0.866 | 0.856 | 0.875 | 0.899 | 0.838 | 0.959 | 0.946 | 0.895 | 0.996 |
| RSAD2_HERC6_IL1R2_RETN_MMP8 | 0.866 | 0.856 | 0.875 | 0.894 | 0.827 | 0.960 | 0.945 | 0.891 | 0.998 |
| RSAD2_HERC6_IL1R2_OLAH_RETN | 0.866 | 0.856 | 0.875 | 0.896 | 0.831 | 0.961 | 0.945 | 0.894 | 0.995 |
| OAS1_IFI44L_HERC6_IL1R2_MMP8 | 0.866 | 0.857 | 0.874 | 0.895 | 0.833 | 0.956 | 0.941 | 0.889 | 0.994 |
| RSAD2_IFI27_OAS1_SIGLEC1_HERC6 | 0.866 | 0.856 | 0.875 | 0.897 | 0.842 | 0.951 | 0.921 | 0.857 | 0.984 |
| OAS1_ISG15_HERC6_IL1R2_OLAH | 0.866 | 0.856 | 0.875 | 0.899 | 0.838 | 0.959 | 0.945 | 0.895 | 0.994 |
| IFI44L_ISG15_HERC6_SLC1A2_IL1R2 | 0.866 | 0.857 | 0.875 | 0.901 | 0.845 | 0.956 | 0.939 | 0.880 | 0.998 |
| OAS1_IFI44L_SIGLEC1_OLAH_MMP8 | 0.866 | 0.856 | 0.875 | 0.892 | 0.823 | 0.960 | 0.955 | 0.912 | 0.999 |
| IFIT1_HERC6_IL1R2_OLAH_RETN | 0.866 | 0.856 | 0.875 | 0.897 | 0.833 | 0.962 | 0.947 | 0.897 | 0.996 |
| IFI44L_ISG15_OLAH_FAM20A_MMP8 | 0.865 | 0.856 | 0.875 | 0.890 | 0.818 | 0.961 | 0.958 | 0.910 | 1 |
| RSAD2_OAS1_SLC1A2_OLAH_MMP8 | 0.865 | 0.856 | 0.875 | 0.900 | 0.842 | 0.959 | 0.939 | 0.885 | 0.994 |
| IFI27_OAS1_SIGLEC1_ISG15_MMP8 | 0.865 | 0.856 | 0.875 | 0.890 | 0.824 | 0.956 | 0.921 | 0.848 | 0.993 |
| IFIT1_ISG15_HERC6_OLAH_RETN | 0.865 | 0.856 | 0.875 | 0.893 | 0.825 | 0.961 | 0.945 | 0.895 | 0.994 |
| OAS1_SLC1A2_IL1R2_OLAH_MMP8 | 0.865 | 0.856 | 0.875 | 0.905 | 0.848 | 0.962 | 0.930 | 0.868 | 0.993 |
| IFI27_OAS1_SIGLEC1_ISG15_SLC1A2 | 0.865 | 0.856 | 0.875 | 0.896 | 0.837 | 0.956 | 0.957 | 0.908 | 1 |
| OAS1_IFIT1_ISG15_SLC1A2_IL1R2 | 0.865 | 0.856 | 0.874 | 0.903 | 0.846 | 0.961 | 0.948 | 0.898 | 0.998 |
| RSAD2_IFI44L_ISG15_SLC1A2_OLAH | 0.865 | 0.856 | 0.875 | 0.906 | 0.849 | 0.964 | 0.935 | 0.879 | 0.990 |
| IFIT1_IFI44L_SLC1A2_IL1R2_MMP8 | 0.865 | 0.857 | 0.874 | 0.904 | 0.848 | 0.960 | 0.953 | 0.907 | 0.999 |
| RSAD2_SIGLEC1_HERC6_RETN_MMP8 | 0.865 | 0.855 | 0.875 | 0.889 | 0.817 | 0.962 | 0.938 | 0.885 | 0.991 |
| OAS1_ISG15_SLC1A2_IL1R2_OLAH | 0.865 | 0.856 | 0.875 | 0.907 | 0.850 | 0.965 | 0.928 | 0.865 | 0.992 |
| IFIT1_IFI44L_HERC6_SLC1A2_IL1R2 | 0.865 | 0.856 | 0.874 | 0.902 | 0.845 | 0.960 | 0.947 | 0.896 | 0.998 |
| RSAD2_ISG15_SLC1A2_IL1R2_OLAH | 0.865 | 0.856 | 0.874 | 0.904 | 0.846 | 0.962 | 0.938 | 0.880 | 0.996 |
| IFI44L_ISG15_SLC1A2_IL1R2_RETN | 0.865 | 0.856 | 0.874 | 0.902 | 0.846 | 0.958 | 0.943 | 0.889 | 0.998 |
| OAS1_HERC6_IL1R2_OLAH_RETN | 0.865 | 0.855 | 0.875 | 0.896 | 0.831 | 0.961 | 0.942 | 0.891 | 0.994 |
| RSAD2_IFIT1_HERC6_IL1R2_OLAH | 0.865 | 0.856 | 0.875 | 0.898 | 0.837 | 0.959 | 0.946 | 0.896 | 0.995 |
| RSAD2_IFI44L_ISG15_HERC6_IL1R2 | 0.865 | 0.856 | 0.874 | 0.896 | 0.838 | 0.955 | 0.943 | 0.892 | 0.995 |
| IFIT1_SIGLEC1_ISG15_OLAH_RETN | 0.865 | 0.856 | 0.875 | 0.892 | 0.822 | 0.961 | 0.946 | 0.891 | 1 |
| IFI27_ISG15_HERC6_SLC1A2_MMP8 | 0.865 | 0.856 | 0.874 | 0.900 | 0.843 | 0.957 | 0.907 | 0.828 | 0.985 |
| IFI44L_SIGLEC1_ISG15_HERC6_RETN | 0.865 | 0.855 | 0.875 | 0.884 | 0.808 | 0.959 | 0.951 | 0.904 | 0.998 |
| IFIT1_SLC1A2_OLAH_RETN_MMP8 | 0.865 | 0.855 | 0.875 | 0.901 | 0.840 | 0.962 | 0.953 | 0.909 | 0.997 |
| RSAD2_IFI27_OAS1_SIGLEC1_MMP8 | 0.865 | 0.855 | 0.875 | 0.890 | 0.825 | 0.956 | 0.921 | 0.848 | 0.993 |
| RSAD2_OAS1_IFI44L_HERC6_IL1R2 | 0.865 | 0.856 | 0.874 | 0.894 | 0.832 | 0.955 | 0.942 | 0.891 | 0.994 |
| RSAD2_IFI27_OAS1_ISG15_FAM20A | 0.865 | 0.855 | 0.875 | 0.883 | 0.809 | 0.957 | 0.940 | 0.861 | 1 |
| IFI27_OAS1_SLC1A2_FAM20A_MMP8 | 0.865 | 0.855 | 0.875 | 0.896 | 0.831 | 0.961 | 0.929 | 0.846 | 1 |
| RSAD2_IFI44L_HERC6_IL1R2_RETN | 0.865 | 0.855 | 0.874 | 0.893 | 0.827 | 0.960 | 0.943 | 0.893 | 0.994 |
| IFI44L_HERC6_SLC1A2_FAM20A_RETN | 0.865 | 0.855 | 0.875 | 0.894 | 0.828 | 0.960 | 0.939 | 0.861 | 1 |
| OAS1_IFI44L_ISG15_HERC6_IL1R2 | 0.865 | 0.856 | 0.874 | 0.897 | 0.839 | 0.955 | 0.946 | 0.895 | 0.996 |
| OAS1_IFIT1_IFI44L_OLAH_FAM20A | 0.865 | 0.855 | 0.875 | 0.890 | 0.818 | 0.963 | 0.949 | 0.893 | 1 |
| IFIT1_ISG15_SLC1A2_IL1R2_RETN | 0.865 | 0.856 | 0.874 | 0.899 | 0.839 | 0.959 | 0.951 | 0.904 | 0.998 |
| IFI27_OAS1_IFIT1_SIGLEC1_HERC6 | 0.865 | 0.856 | 0.874 | 0.898 | 0.843 | 0.953 | 0.933 | 0.876 | 0.989 |
| ISG15_HERC6_SLC1A2_IL1R2_MMP8 | 0.865 | 0.856 | 0.874 | 0.901 | 0.845 | 0.956 | 0.932 | 0.866 | 0.997 |
| IFI44L_SIGLEC1_ISG15_OLAH_RETN | 0.865 | 0.855 | 0.875 | 0.890 | 0.820 | 0.961 | 0.948 | 0.896 | 1.000 |
| IFIT1_ISG15_SLC1A2_IL1R2_MMP8 | 0.865 | 0.857 | 0.873 | 0.904 | 0.848 | 0.961 | 0.952 | 0.906 | 0.999 |
| ISG15_SLC1A2_OLAH_FAM20A_RETN | 0.865 | 0.855 | 0.875 | 0.903 | 0.844 | 0.962 | 0.913 | 0.831 | 0.995 |
| RSAD2_IFI27_SLC1A2_FAM20A_MMP8 | 0.865 | 0.855 | 0.875 | 0.896 | 0.831 | 0.960 | 0.932 | 0.850 | 1 |
| RSAD2_SIGLEC1_OLAH_RETN_MMP8 | 0.865 | 0.855 | 0.875 | 0.892 | 0.822 | 0.962 | 0.963 | 0.922 | 1 |
| RSAD2_IFIT1_IFI44L_SIGLEC1_OLAH | 0.865 | 0.856 | 0.874 | 0.895 | 0.827 | 0.962 | 0.948 | 0.897 | 0.999 |
| RSAD2_OAS1_HERC6_SLC1A2_IL1R2 | 0.865 | 0.856 | 0.874 | 0.898 | 0.841 | 0.955 | 0.935 | 0.870 | 1.000 |
| IFIT1_IFI44L_HERC6_OLAH_RETN | 0.865 | 0.855 | 0.874 | 0.892 | 0.823 | 0.961 | 0.938 | 0.884 | 0.992 |
| RSAD2_IFI27_OAS1_IFIT1_FAM20A | 0.865 | 0.855 | 0.875 | 0.887 | 0.816 | 0.958 | 0.942 | 0.764 | 1 |
| IFI44L_HERC6_SLC1A2_IL1R2_MMP8 | 0.865 | 0.856 | 0.874 | 0.900 | 0.844 | 0.955 | 0.933 | 0.871 | 0.994 |
| RSAD2_IFI27_IFI44L_SIGLEC1_MMP8 | 0.865 | 0.855 | 0.874 | 0.891 | 0.825 | 0.957 | 0.915 | 0.842 | 0.988 |
| OAS1_OLAH_FAM20A_RETN_MMP8 | 0.865 | 0.854 | 0.875 | 0.891 | 0.820 | 0.963 | 0.957 | 0.901 | 1 |
| RSAD2_IFI27_SIGLEC1_ISG15_HERC6 | 0.865 | 0.855 | 0.874 | 0.894 | 0.838 | 0.950 | 0.920 | 0.854 | 0.985 |
| IFI27_ISG15_SLC1A2_FAM20A_MMP8 | 0.865 | 0.855 | 0.875 | 0.896 | 0.830 | 0.962 | 0.925 | 0.842 | 1 |
| RSAD2_IFI44L_HERC6_IL1R2_OLAH | 0.865 | 0.855 | 0.874 | 0.897 | 0.836 | 0.958 | 0.947 | 0.897 | 0.996 |
| ISG15_HERC6_OLAH_RETN_MMP8 | 0.865 | 0.854 | 0.875 | 0.892 | 0.822 | 0.962 | 0.961 | 0.922 | 1.000 |
| RSAD2_ISG15_HERC6_OLAH_RETN | 0.865 | 0.855 | 0.874 | 0.894 | 0.828 | 0.961 | 0.946 | 0.897 | 0.994 |
| IFIT1_IFI44L_ISG15_SLC1A2_IL1R2 | 0.865 | 0.856 | 0.873 | 0.900 | 0.842 | 0.958 | 0.948 | 0.898 | 0.998 |
| IFI27_OAS1_IFI44L_SIGLEC1_SLC1A2 | 0.865 | 0.856 | 0.873 | 0.896 | 0.836 | 0.955 | 0.953 | 0.903 | 1 |
| IFI44L_ISG15_HERC6_OLAH_RETN | 0.864 | 0.855 | 0.874 | 0.891 | 0.820 | 0.961 | 0.953 | 0.909 | 0.998 |
| RSAD2_IFIT1_SLC1A2_IL1R2_MMP8 | 0.864 | 0.856 | 0.873 | 0.902 | 0.844 | 0.960 | 0.954 | 0.909 | 1.000 |
| IFIT1_HERC6_OLAH_RETN_MMP8 | 0.864 | 0.854 | 0.875 | 0.892 | 0.822 | 0.961 | 0.958 | 0.917 | 0.999 |
| IFI44L_HERC6_OLAH_RETN_MMP8 | 0.864 | 0.854 | 0.875 | 0.890 | 0.818 | 0.961 | 0.958 | 0.916 | 0.999 |
| IFI27_OAS1_IFI44L_SIGLEC1_HERC6 | 0.864 | 0.855 | 0.873 | 0.897 | 0.842 | 0.953 | 0.932 | 0.874 | 0.989 |
| OAS1_SLC1A2_IL1R2_OLAH_RETN | 0.864 | 0.855 | 0.874 | 0.907 | 0.849 | 0.964 | 0.932 | 0.870 | 0.993 |
| OAS1_HERC6_SLC1A2_IL1R2_MMP8 | 0.864 | 0.855 | 0.873 | 0.900 | 0.843 | 0.957 | 0.925 | 0.857 | 0.993 |
| RSAD2_HERC6_SLC1A2_IL1R2_MMP8 | 0.864 | 0.855 | 0.873 | 0.898 | 0.840 | 0.956 | 0.933 | 0.867 | 0.999 |
| OAS1_HERC6_SLC1A2_IL1R2_RETN | 0.864 | 0.855 | 0.874 | 0.899 | 0.840 | 0.958 | 0.922 | 0.849 | 0.994 |
| OAS1_IFI44L_SIGLEC1_OLAH_RETN | 0.864 | 0.854 | 0.874 | 0.891 | 0.820 | 0.961 | 0.952 | 0.905 | 1.000 |
| OAS1_IFI44L_OLAH_FAM20A_MMP8 | 0.864 | 0.854 | 0.875 | 0.891 | 0.819 | 0.962 | 0.953 | 0.898 | 1 |
| RSAD2_IFI44L_IL1R2_FAM20A_RETN | 0.864 | 0.855 | 0.874 | 0.894 | 0.828 | 0.960 | 0.945 | 0.873 | 1 |
| RSAD2_IFIT1_SIGLEC1_ISG15_OLAH | 0.864 | 0.855 | 0.873 | 0.894 | 0.827 | 0.962 | 0.947 | 0.896 | 0.998 |
| ISG15_SLC1A2_IL1R2_OLAH_RETN | 0.864 | 0.855 | 0.873 | 0.899 | 0.840 | 0.957 | 0.921 | 0.846 | 0.995 |
| OAS1_HERC6_OLAH_RETN_MMP8 | 0.864 | 0.854 | 0.874 | 0.892 | 0.822 | 0.961 | 0.955 | 0.913 | 0.998 |
| IFIT1_SIGLEC1_HERC6_SLC1A2_MMP8 | 0.864 | 0.855 | 0.873 | 0.893 | 0.833 | 0.954 | 0.909 | 0.838 | 0.980 |
| OAS1_ISG15_OLAH_FAM20A_RETN | 0.864 | 0.854 | 0.874 | 0.893 | 0.822 | 0.964 | 0.952 | 0.894 | 1 |
| OAS1_IFI44L_HERC6_FAM20A_RETN | 0.864 | 0.854 | 0.874 | 0.892 | 0.822 | 0.962 | 0.942 | 0.867 | 1 |
| RSAD2_IFI27_IFIT1_ISG15_FAM20A | 0.864 | 0.854 | 0.874 | 0.890 | 0.820 | 0.960 | 0.940 | 0.861 | 1 |
| RSAD2_IFI44L_HERC6_IL1R2_MMP8 | 0.864 | 0.855 | 0.873 | 0.894 | 0.833 | 0.955 | 0.942 | 0.891 | 0.994 |
| OAS1_ISG15_OLAH_FAM20A_MMP8 | 0.864 | 0.854 | 0.874 | 0.894 | 0.824 | 0.965 | 0.950 | 0.886 | 1 |
| OAS1_ISG15_SLC1A2_OLAH_MMP8 | 0.864 | 0.855 | 0.873 | 0.902 | 0.844 | 0.960 | 0.932 | 0.872 | 0.991 |
| OAS1_IFIT1_SLC1A2_IL1R2_RETN | 0.864 | 0.855 | 0.873 | 0.899 | 0.839 | 0.959 | 0.949 | 0.900 | 0.997 |
| RSAD2_IFIT1_SLC1A2_OLAH_RETN | 0.864 | 0.854 | 0.873 | 0.905 | 0.846 | 0.965 | 0.945 | 0.894 | 0.995 |
| RSAD2_OAS1_HERC6_FAM20A_RETN | 0.864 | 0.854 | 0.874 | 0.886 | 0.815 | 0.957 | 0.934 | 0.855 | 1 |
| RSAD2_IFI44L_HERC6_FAM20A_RETN | 0.864 | 0.854 | 0.874 | 0.895 | 0.828 | 0.962 | 0.929 | 0.850 | 1 |
| OAS1_IFIT1_HERC6_FAM20A_RETN | 0.864 | 0.854 | 0.874 | 0.889 | 0.818 | 0.960 | 0.936 | 0.857 | 1 |
| RSAD2_HERC6_OLAH_RETN_MMP8 | 0.864 | 0.854 | 0.874 | 0.890 | 0.821 | 0.960 | 0.954 | 0.911 | 0.998 |
| OAS1_IFIT1_HERC6_OLAH_RETN | 0.864 | 0.854 | 0.874 | 0.890 | 0.820 | 0.959 | 0.940 | 0.888 | 0.993 |
| RSAD2_IFI27_OAS1_SIGLEC1_SLC1A2 | 0.864 | 0.854 | 0.873 | 0.896 | 0.837 | 0.955 | 0.951 | 0.900 | 1 |
| RSAD2_IFIT1_ISG15_SLC1A2_OLAH | 0.864 | 0.854 | 0.873 | 0.902 | 0.843 | 0.961 | 0.943 | 0.893 | 0.994 |
| IFI27_IFIT1_SIGLEC1_ISG15_SLC1A2 | 0.864 | 0.855 | 0.873 | 0.895 | 0.836 | 0.954 | 0.953 | 0.905 | 1 |
| RSAD2_HERC6_SLC1A2_IL1R2_RETN | 0.864 | 0.854 | 0.873 | 0.899 | 0.840 | 0.958 | 0.932 | 0.865 | 0.998 |
| RSAD2_IFIT1_ISG15_SLC1A2_IL1R2 | 0.864 | 0.855 | 0.872 | 0.900 | 0.842 | 0.959 | 0.948 | 0.898 | 0.998 |
| OAS1_IFI44L_HERC6_SLC1A2_IL1R2 | 0.864 | 0.854 | 0.873 | 0.900 | 0.844 | 0.956 | 0.935 | 0.874 | 0.996 |
| RSAD2_OAS1_HERC6_OLAH_RETN | 0.863 | 0.854 | 0.873 | 0.889 | 0.819 | 0.959 | 0.942 | 0.891 | 0.993 |
| RSAD2_ISG15_SLC1A2_OLAH_MMP8 | 0.863 | 0.854 | 0.873 | 0.900 | 0.842 | 0.959 | 0.936 | 0.880 | 0.992 |
| IFIT1_IFI44L_SLC1A2_IL1R2_RETN | 0.863 | 0.854 | 0.873 | 0.899 | 0.840 | 0.959 | 0.945 | 0.893 | 0.996 |
| RSAD2_IFI27_IFIT1_SIGLEC1_MMP8 | 0.863 | 0.854 | 0.873 | 0.889 | 0.822 | 0.956 | 0.911 | 0.835 | 0.987 |
| RSAD2_OAS1_IFIT1_SLC1A2_IL1R2 | 0.863 | 0.854 | 0.872 | 0.900 | 0.841 | 0.959 | 0.949 | 0.899 | 0.999 |
| IFI27_OAS1_IFIT1_ISG15_FAM20A | 0.863 | 0.853 | 0.874 | 0.886 | 0.815 | 0.957 | 0.945 | 0.866 | 1 |
| IFIT1_IFI44L_OLAH_FAM20A_MMP8 | 0.863 | 0.853 | 0.873 | 0.888 | 0.817 | 0.959 | 0.958 | 0.911 | 1 |
| RSAD2_OAS1_SLC1A2_OLAH_RETN | 0.863 | 0.854 | 0.873 | 0.903 | 0.844 | 0.962 | 0.940 | 0.866 | 0.994 |
| IFI44L_ISG15_SLC1A2_IL1R2_MMP8 | 0.863 | 0.855 | 0.871 | 0.899 | 0.843 | 0.954 | 0.939 | 0.878 | 1 |
| RSAD2_OAS1_SIGLEC1_ISG15_OLAH | 0.863 | 0.854 | 0.873 | 0.892 | 0.823 | 0.960 | 0.945 | 0.893 | 0.996 |
| OAS1_IFI44L_ISG15_OLAH_FAM20A | 0.863 | 0.853 | 0.873 | 0.890 | 0.817 | 0.963 | 0.953 | 0.898 | 1 |
| IFI27_IFIT1_ISG15_SLC1A2_FAM20A | 0.863 | 0.853 | 0.874 | 0.889 | 0.820 | 0.958 | 0.941 | 0.862 | 1 |
| IFI44L_SIGLEC1_HERC6_SLC1A2_RETN | 0.863 | 0.854 | 0.872 | 0.893 | 0.827 | 0.960 | 0.951 | 0.903 | 0.999 |
| IFI27_IFI44L_SIGLEC1_ISG15_SLC1A2 | 0.863 | 0.854 | 0.872 | 0.895 | 0.835 | 0.955 | 0.953 | 0.904 | 1 |
| IFI44L_ISG15_OLAH_FAM20A_RETN | 0.863 | 0.853 | 0.873 | 0.891 | 0.821 | 0.962 | 0.958 | 0.912 | 1 |
| RSAD2_ISG15_SLC1A2_OLAH_RETN | 0.863 | 0.853 | 0.873 | 0.903 | 0.844 | 0.961 | 0.941 | 0.888 | 0.995 |
| OAS1_ISG15_SLC1A2_OLAH_RETN | 0.863 | 0.853 | 0.873 | 0.903 | 0.845 | 0.962 | 0.937 | 0.880 | 0.994 |
| RSAD2_IFI27_IFI44L_SIGLEC1_HERC6 | 0.863 | 0.854 | 0.872 | 0.896 | 0.840 | 0.951 | 0.912 | 0.843 | 0.981 |
| IFIT1_HERC6_SLC1A2_FAM20A_RETN | 0.863 | 0.853 | 0.873 | 0.897 | 0.832 | 0.961 | 0.939 | 0.858 | 1 |
| RSAD2_IFI44L_HERC6_SLC1A2_IL1R2 | 0.863 | 0.854 | 0.872 | 0.900 | 0.843 | 0.956 | 0.936 | 0.874 | 0.998 |
| RSAD2_IFI44L_SIGLEC1_OLAH_MMP8 | 0.863 | 0.853 | 0.873 | 0.891 | 0.823 | 0.959 | 0.957 | 0.914 | 0.999 |
| OAS1_IFIT1_IL1R2_OLAH_MMP8 | 0.863 | 0.853 | 0.872 | 0.894 | 0.831 | 0.958 | 0.960 | 0.920 | 1.000 |
| RSAD2_OAS1_ISG15_SLC1A2_OLAH | 0.863 | 0.853 | 0.872 | 0.903 | 0.845 | 0.961 | 0.934 | 0.876 | 0.992 |
| RSAD2_IFI27_SIGLEC1_ISG15_MMP8 | 0.863 | 0.853 | 0.872 | 0.888 | 0.821 | 0.955 | 0.914 | 0.840 | 0.988 |
| IFI27_IFIT1_SIGLEC1_HERC6_SLC1A2 | 0.863 | 0.854 | 0.872 | 0.898 | 0.840 | 0.955 | 0.950 | 0.902 | 0.998 |
| RSAD2_SLC1A2_OLAH_RETN_MMP8 | 0.863 | 0.853 | 0.872 | 0.900 | 0.840 | 0.960 | 0.946 | 0.895 | 0.996 |
| ISG15_SLC1A2_IL1R2_OLAH_MMP8 | 0.863 | 0.854 | 0.872 | 0.896 | 0.838 | 0.954 | 0.926 | 0.856 | 0.997 |
| RSAD2_OAS1_IFI44L_SIGLEC1_OLAH | 0.863 | 0.853 | 0.872 | 0.892 | 0.824 | 0.961 | 0.946 | 0.895 | 0.996 |
| IFIT1_SLC1A2_IL1R2_RETN_MMP8 | 0.863 | 0.853 | 0.872 | 0.903 | 0.845 | 0.961 | 0.959 | 0.917 | 1 |
| OAS1_IFI44L_SIGLEC1_ISG15_OLAH | 0.863 | 0.853 | 0.872 | 0.892 | 0.824 | 0.961 | 0.943 | 0.892 | 0.995 |
| OAS1_SIGLEC1_ISG15_OLAH_RETN | 0.863 | 0.852 | 0.873 | 0.890 | 0.820 | 0.961 | 0.949 | 0.898 | 1 |
| OAS1_IFIT1_IFI44L_SLC1A2_IL1R2 | 0.863 | 0.853 | 0.872 | 0.901 | 0.843 | 0.959 | 0.948 | 0.898 | 0.997 |
| IFI44L_OLAH_FAM20A_RETN_MMP8 | 0.863 | 0.852 | 0.873 | 0.894 | 0.826 | 0.962 | 0.960 | 0.917 | 1 |
| SIGLEC1_ISG15_HERC6_SLC1A2_RETN | 0.862 | 0.853 | 0.872 | 0.893 | 0.826 | 0.960 | 0.960 | 0.916 | 1 |
| OAS1_SIGLEC1_ISG15_OLAH_MMP8 | 0.862 | 0.853 | 0.872 | 0.890 | 0.821 | 0.959 | 0.951 | 0.904 | 0.998 |
| OAS1_IFI44L_HERC6_OLAH_RETN | 0.862 | 0.852 | 0.872 | 0.890 | 0.820 | 0.960 | 0.946 | 0.897 | 0.995 |
| RSAD2_SIGLEC1_ISG15_OLAH_MMP8 | 0.862 | 0.853 | 0.872 | 0.891 | 0.823 | 0.959 | 0.952 | 0.906 | 0.998 |
| HERC6_SLC1A2_IL1R2_RETN_MMP8 | 0.862 | 0.853 | 0.872 | 0.899 | 0.841 | 0.957 | 0.922 | 0.847 | 0.997 |
| RSAD2_OAS1_IFIT1_IL1R2_OLAH | 0.862 | 0.853 | 0.872 | 0.893 | 0.826 | 0.960 | 0.946 | 0.896 | 0.995 |
| RSAD2_OAS1_IFIT1_IL1R2_MMP8 | 0.862 | 0.853 | 0.872 | 0.892 | 0.827 | 0.956 | 0.954 | 0.911 | 0.998 |
| OAS1_HERC6_SLC1A2_FAM20A_RETN | 0.862 | 0.852 | 0.872 | 0.896 | 0.832 | 0.961 | 0.937 | 0.856 | 1 |
| IFI27_OAS1_ISG15_SLC1A2_FAM20A | 0.862 | 0.852 | 0.873 | 0.887 | 0.817 | 0.957 | 0.936 | 0.854 | 1 |
| IFI27_IFI44L_ISG15_SLC1A2_MMP8 | 0.862 | 0.853 | 0.872 | 0.894 | 0.832 | 0.956 | 0.915 | 0.843 | 0.987 |
| IFIT1_IFI44L_ISG15_OLAH_FAM20A | 0.862 | 0.852 | 0.872 | 0.889 | 0.817 | 0.961 | 0.957 | 0.910 | 1 |
| RSAD2_IFI27_SIGLEC1_ISG15_SLC1A2 | 0.862 | 0.853 | 0.871 | 0.895 | 0.836 | 0.955 | 0.952 | 0.903 | 1 |
| OAS1_IFI44L_OLAH_FAM20A_RETN | 0.862 | 0.852 | 0.872 | 0.891 | 0.818 | 0.963 | 0.954 | 0.901 | 1 |
| RSAD2_SIGLEC1_ISG15_OLAH_RETN | 0.862 | 0.852 | 0.872 | 0.890 | 0.819 | 0.961 | 0.950 | 0.899 | 1 |
| RSAD2_IFI44L_SIGLEC1_OLAH_RETN | 0.862 | 0.852 | 0.872 | 0.890 | 0.819 | 0.961 | 0.950 | 0.901 | 0.999 |
| RSAD2_IFIT1_SLC1A2_IL1R2_RETN | 0.862 | 0.853 | 0.871 | 0.896 | 0.835 | 0.957 | 0.946 | 0.893 | 0.998 |
| OAS1_IFIT1_IFI44L_IL1R2_MMP8 | 0.862 | 0.853 | 0.871 | 0.892 | 0.828 | 0.957 | 0.954 | 0.911 | 0.998 |
| OAS1_IFIT1_SIGLEC1_SLC1A2_RETN | 0.862 | 0.853 | 0.871 | 0.894 | 0.832 | 0.957 | 0.947 | 0.897 | 0.996 |
| IFI44L_SLC1A2_IL1R2_RETN_MMP8 | 0.862 | 0.853 | 0.871 | 0.901 | 0.846 | 0.957 | 0.943 | 0.885 | 1 |
| OAS1_SLC1A2_OLAH_RETN_MMP8 | 0.862 | 0.852 | 0.871 | 0.903 | 0.844 | 0.962 | 0.940 | 0.886 | 0.994 |
| RSAD2_IFI27_IFI44L_SIGLEC1_SLC1A2 | 0.862 | 0.853 | 0.871 | 0.897 | 0.838 | 0.955 | 0.852 | 0.904 | 1 |
| OAS1_IFI44L_ISG15_HERC6_RETN | 0.862 | 0.851 | 0.872 | 0.891 | 0.823 | 0.960 | 0.936 | 0.877 | 0.995 |
| RSAD2_HERC6_SLC1A2_FAM20A_RETN | 0.862 | 0.852 | 0.872 | 0.897 | 0.832 | 0.962 | 0.939 | 0.858 | 1 |
| OAS1_IFIT1_ISG15_HERC6_RETN | 0.862 | 0.851 | 0.872 | 0.890 | 0.822 | 0.959 | 0.939 | 0.883 | 0.995 |
| OAS1_ISG15_HERC6_SLC1A2_RETN | 0.862 | 0.852 | 0.872 | 0.895 | 0.831 | 0.958 | 0.936 | 0.871 | 1 |
| OAS1_IFIT1_ISG15_IL1R2_OLAH | 0.862 | 0.852 | 0.871 | 0.893 | 0.826 | 0.961 | 0.946 | 0.896 | 0.995 |
| IFI44L_HERC6_FAM20A_RETN_MMP8 | 0.862 | 0.851 | 0.872 | 0.893 | 0.822 | 0.965 | 0.943 | 0.868 | 1 |
| RSAD2_IFI27_IFIT1_SIGLEC1_HERC6 | 0.862 | 0.852 | 0.871 | 0.896 | 0.840 | 0.951 | 0.917 | 0.852 | 0.982 |
| IFIT1_ISG15_IL1R2_OLAH_MMP8 | 0.862 | 0.852 | 0.871 | 0.893 | 0.831 | 0.955 | 0.965 | 0.929 | 1 |
| OAS1_SIGLEC1_HERC6_SLC1A2_MMP8 | 0.862 | 0.853 | 0.871 | 0.889 | 0.825 | 0.952 | 0.936 | 0.879 | 0.993 |
| RSAD2_IFI27_ISG15_SLC1A2_FAM20A | 0.862 | 0.851 | 0.872 | 0.887 | 0.817 | 0.957 | 0.938 | 0.858 | 1 |
| RSAD2_IFIT1_IFI44L_SLC1A2_IL1R2 | 0.861 | 0.853 | 0.870 | 0.900 | 0.841 | 0.958 | 0.947 | 0.896 | 0.997 |
| IFIT1_IFI44L_IL1R2_OLAH_MMP8 | 0.861 | 0.852 | 0.871 | 0.890 | 0.826 | 0.954 | 0.961 | 0.922 | 1 |
| RSAD2_IFI44L_HERC6_OLAH_RETN | 0.861 | 0.851 | 0.871 | 0.891 | 0.822 | 0.960 | 0.941 | 0.890 | 0.993 |
| RSAD2_IFIT1_HERC6_FAM20A_RETN | 0.861 | 0.851 | 0.871 | 0.886 | 0.815 | 0.958 | 0.938 | 0.860 | 1 |
| RSAD2_SIGLEC1_HERC6_SLC1A2_MMP8 | 0.861 | 0.852 | 0.870 | 0.890 | 0.826 | 0.953 | 0.927 | 0.867 | 0.987 |
| RSAD2_IFI27_IFIT1_SLC1A2_FAM20A | 0.861 | 0.851 | 0.872 | 0.889 | 0.820 | 0.958 | 0.940 | 0.861 | 1 |
| RSAD2_OAS1_IFIT1_IFI44L_IL1R2 | 0.861 | 0.852 | 0.871 | 0.889 | 0.821 | 0.956 | 0.948 | 0.901 | 0.995 |
| IFIT1_IFI44L_HERC6_OLAH_MMP8 | 0.861 | 0.851 | 0.871 | 0.889 | 0.823 | 0.955 | 0.939 | 0.885 | 0.993 |
| OAS1_IFIT1_IL1R2_OLAH_RETN | 0.861 | 0.851 | 0.871 | 0.892 | 0.823 | 0.961 | 0.949 | 0.901 | 0.996 |
| IFI44L_SIGLEC1_HERC6_RETN_MMP8 | 0.861 | 0.851 | 0.871 | 0.883 | 0.808 | 0.959 | 0.943 | 0.894 | 0.993 |
| RSAD2_IFI27_IFIT1_SIGLEC1_SLC1A2 | 0.861 | 0.852 | 0.870 | 0.896 | 0.836 | 0.955 | 0.950 | 0.900 | 1.000 |
| OAS1_IFIT1_IFI44L_IL1R2_OLAH | 0.861 | 0.851 | 0.870 | 0.894 | 0.827 | 0.961 | 0.946 | 0.896 | 0.995 |
| IFI44L_ISG15_IL1R2_OLAH_MMP8 | 0.861 | 0.852 | 0.870 | 0.893 | 0.835 | 0.952 | 0.958 | 0.915 | 1.000 |
| RSAD2_OAS1_IL1R2_OLAH_MMP8 | 0.861 | 0.851 | 0.870 | 0.894 | 0.831 | 0.957 | 0.941 | 0.887 | 0.996 |
| RSAD2_IFIT1_HERC6_OLAH_RETN | 0.861 | 0.851 | 0.870 | 0.889 | 0.820 | 0.959 | 0.940 | 0.888 | 0.993 |
| IFI27_IFI44L_SIGLEC1_HERC6_SLC1A2 | 0.861 | 0.852 | 0.870 | 0.893 | 0.832 | 0.954 | 0.953 | 0.904 | 1 |
| RSAD2_OAS1_ISG15_HERC6_RETN | 0.861 | 0.851 | 0.871 | 0.890 | 0.821 | 0.958 | 0.941 | 0.887 | 0.996 |
| HERC6_SLC1A2_FAM20A_RETN_MMP8 | 0.861 | 0.851 | 0.871 | 0.896 | 0.832 | 0.960 | 0.936 | 0.854 | 1 |
| RSAD2_IFI44L_HERC6_SLC1A2_FAM20A | 0.861 | 0.851 | 0.871 | 0.895 | 0.834 | 0.955 | 0.922 | 0.838 | 1 |
| IFIT1_IFI44L_ISG15_IL1R2_MMP8 | 0.861 | 0.852 | 0.870 | 0.894 | 0.832 | 0.956 | 0.962 | 0.923 | 1 |
| RSAD2_IFI44L_SIGLEC1_ISG15_OLAH | 0.861 | 0.851 | 0.870 | 0.892 | 0.823 | 0.960 | 0.950 | 0.902 | 0.998 |
| SIGLEC1_HERC6_SLC1A2_RETN_MMP8 | 0.861 | 0.851 | 0.870 | 0.893 | 0.827 | 0.960 | 0.955 | 0.910 | 1 |
| IFI27_OAS1_IFI44L_SLC1A2_MMP8 | 0.861 | 0.851 | 0.870 | 0.893 | 0.832 | 0.955 | 0.912 | 0.838 | 0.986 |
| OAS1_IFIT1_ISG15_IL1R2_RETN | 0.861 | 0.851 | 0.870 | 0.890 | 0.821 | 0.959 | 0.954 | 0.911 | 0.998 |
| RSAD2_OAS1_HERC6_OLAH_MMP8 | 0.861 | 0.851 | 0.870 | 0.888 | 0.821 | 0.954 | 0.936 | 0.880 | 0.991 |
| RSAD2_IFI44L_ISG15_HERC6_RETN | 0.861 | 0.850 | 0.871 | 0.892 | 0.824 | 0.960 | 0.933 | 0.875 | 0.991 |
| IFI44L_ISG15_SLC1A2_FAM20A_RETN | 0.861 | 0.851 | 0.871 | 0.893 | 0.827 | 0.959 | 0.935 | 0.860 | 1 |
| IFIT1_ISG15_HERC6_OLAH_MMP8 | 0.861 | 0.851 | 0.871 | 0.888 | 0.822 | 0.954 | 0.943 | 0.894 | 0.993 |
| RSAD2_IFI44L_ISG15_HERC6_FAM20A | 0.861 | 0.851 | 0.871 | 0.890 | 0.825 | 0.955 | 0.824 | 0.844 | 1 |
| IFI27_OAS1_IFIT1_SLC1A2_FAM20A | 0.860 | 0.850 | 0.871 | 0.888 | 0.819 | 0.957 | 0.941 | 0.862 | 1 |
| OAS1_IFIT1_IL1R2_RETN_MMP8 | 0.860 | 0.851 | 0.870 | 0.889 | 0.819 | 0.958 | 0.963 | 0.925 | 1 |
| RSAD2_IFI27_OAS1_SLC1A2_FAM20A | 0.860 | 0.850 | 0.871 | 0.889 | 0.820 | 0.958 | 0.936 | 0.854 | 1 |
| OAS1_IFIT1_HERC6_OLAH_MMP8 | 0.860 | 0.851 | 0.870 | 0.887 | 0.819 | 0.954 | 0.940 | 0.887 | 0.993 |
| RSAD2_OAS1_ISG15_IL1R2_OLAH | 0.860 | 0.851 | 0.870 | 0.895 | 0.829 | 0.961 | 0.948 | 0.898 | 0.997 |
| OAS1_IFIT1_ISG15_IL1R2_MMP8 | 0.860 | 0.851 | 0.869 | 0.890 | 0.826 | 0.954 | 0.959 | 0.918 | 0.999 |
| OAS1_IFIT1_SIGLEC1_HERC6_SLC1A2 | 0.860 | 0.851 | 0.869 | 0.889 | 0.828 | 0.949 | 0.930 | 0.873 | 0.988 |
| IFI27_SIGLEC1_ISG15_HERC6_SLC1A2 | 0.860 | 0.851 | 0.870 | 0.891 | 0.830 | 0.952 | 0.959 | 0.912 | 1 |
| IFIT1_IFI44L_SIGLEC1_SLC1A2_RETN | 0.860 | 0.851 | 0.869 | 0.895 | 0.832 | 0.957 | 0.940 | 0.886 | 0.994 |
| RSAD2_OAS1_IL1R2_OLAH_RETN | 0.860 | 0.851 | 0.870 | 0.893 | 0.825 | 0.961 | 0.943 | 0.891 | 0.996 |
| OAS1_IFIT1_IFI44L_ISG15_IL1R2 | 0.860 | 0.851 | 0.869 | 0.891 | 0.825 | 0.957 | 0.949 | 0.902 | 0.996 |
| RSAD2_OAS1_IFIT1_IL1R2_RETN | 0.860 | 0.851 | 0.870 | 0.887 | 0.816 | 0.958 | 0.951 | 0.906 | 0.997 |
| RSAD2_OAS1_IFIT1_ISG15_IL1R2 | 0.860 | 0.851 | 0.869 | 0.891 | 0.825 | 0.957 | 0.949 | 0.902 | 0.996 |
| IFIT1_IL1R2_OLAH_RETN_MMP8 | 0.860 | 0.850 | 0.870 | 0.893 | 0.827 | 0.960 | 0.964 | 0.927 | 1 |
| IFI44L_ISG15_HERC6_OLAH_MMP8 | 0.860 | 0.850 | 0.870 | 0.888 | 0.820 | 0.955 | 0.947 | 0.898 | 0.995 |
| IFIT1_IFI44L_ISG15_IL1R2_OLAH | 0.860 | 0.851 | 0.869 | 0.894 | 0.830 | 0.958 | 0.959 | 0.918 | 1.000 |
| IFIT1_HERC6_FAM20A_RETN_MMP8 | 0.860 | 0.849 | 0.870 | 0.888 | 0.818 | 0.959 | 0.940 | 0.863 | 1 |
| OAS1_IFIT1_IFI44L_IL1R2_RETN | 0.860 | 0.850 | 0.870 | 0.888 | 0.817 | 0.958 | 0.950 | 0.904 | 0.996 |
| RSAD2_IFI27_IFI44L_SLC1A2_MMP8 | 0.860 | 0.850 | 0.870 | 0.893 | 0.831 | 0.955 | 0.915 | 0.842 | 0.988 |
| IFIT1_SIGLEC1_ISG15_SLC1A2_RETN | 0.860 | 0.851 | 0.869 | 0.895 | 0.833 | 0.957 | 0.949 | 0.900 | 0.998 |
| IFI27_IFIT1_ISG15_SLC1A2_MMP8 | 0.860 | 0.850 | 0.869 | 0.889 | 0.826 | 0.953 | 0.920 | 0.847 | 0.992 |
| RSAD2_IFIT1_IL1R2_OLAH_MMP8 | 0.860 | 0.850 | 0.869 | 0.889 | 0.825 | 0.953 | 0.963 | 0.925 | 1 |
| OAS1_SIGLEC1_ISG15_SLC1A2_RETN | 0.860 | 0.850 | 0.869 | 0.893 | 0.830 | 0.857 | 0.951 | 0.902 | 1 |
| IFI27_IFIT1_IFI44L_SIGLEC1_SLC1A2 | 0.860 | 0.850 | 0.869 | 0.893 | 0.831 | 0.954 | 0.955 | 0.908 | 1 |
| RSAD2_OAS1_IFI44L_IL1R2_OLAH | 0.860 | 0.850 | 0.869 | 0.894 | 0.829 | 0.960 | 0.941 | 0.887 | 0.995 |
| RSAD2_IFI44L_ISG15_OLAH_FAM20A | 0.860 | 0.849 | 0.870 | 0.890 | 0.818 | 0.961 | 0.958 | 0.910 | 1 |
| IFI27_IFI44L_SIGLEC1_ISG15_HERC6 | 0.859 | 0.850 | 0.869 | 0.892 | 0.832 | 0.952 | 0.926 | 0.866 | 0.986 |
| RSAD2_ISG15_SLC1A2_IL1R2_RETN | 0.859 | 0.850 | 0.868 | 0.896 | 0.837 | 0.955 | 0.945 | 0.886 | 1 |
| IFI27_OAS1_IFIT1_SLC1A2_MMP8 | 0.859 | 0.850 | 0.869 | 0.889 | 0.826 | 0.953 | 0.920 | 0.846 | 0.993 |
| RSAD2_OAS1_ISG15_HERC6_OLAH | 0.859 | 0.850 | 0.869 | 0.886 | 0.821 | 0.951 | 0.945 | 0.895 | 0.994 |
| SIGLEC1_ISG15_HERC6_RETN_MMP8 | 0.859 | 0.849 | 0.870 | 0.879 | 0.800 | 0.958 | 0.959 | 0.916 | 1 |
| RSAD2_IFIT1_IFI44L_IL1R2_MMP8 | 0.859 | 0.850 | 0.868 | 0.890 | 0.826 | 0.955 | 0.958 | 0.916 | 0.999 |
| IFIT1_IFI44L_IL1R2_RETN_MMP8 | 0.859 | 0.849 | 0.869 | 0.889 | 0.820 | 0.959 | 0.965 | 0.929 | 1 |
| OAS1_IFI44L_SIGLEC1_SLC1A2_RETN | 0.859 | 0.850 | 0.868 | 0.893 | 0.830 | 0.957 | 0.948 | 0.897 | 0.999 |
| OAS1_IFI44L_IL1R2_OLAH_MMP8 | 0.859 | 0.850 | 0.868 | 0.895 | 0.832 | 0.957 | 0.946 | 0.894 | 0.997 |
| RSAD2_IFI44L_ISG15_SLC1A2_IL1R2 | 0.859 | 0.851 | 0.868 | 0.897 | 0.839 | 0.954 | 0.943 | 0.888 | 0.999 |
| OAS1_ISG15_IL1R2_OLAH_MMP8 | 0.859 | 0.850 | 0.868 | 0.894 | 0.831 | 0.957 | 0.939 | 0.883 | 0.996 |
| RSAD2_IFI27_OAS1_SLC1A2_MMP8 | 0.859 | 0.849 | 0.869 | 0.889 | 0.826 | 0.953 | 0.913 | 0.835 | 0.991 |
| RSAD2_OAS1_SIGLEC1_SLC1A2_RETN | 0.859 | 0.849 | 0.869 | 0.893 | 0.829 | 0.956 | 0.953 | 0.906 | 1 |
| IFIT1_SIGLEC1_ISG15_HERC6_MMP8 | 0.859 | 0.850 | 0.868 | 0.886 | 0.820 | 0.952 | 0.895 | 0.816 | 0.973 |
| IFI27_IFIT1_IFI44L_SLC1A2_MMP8 | 0.859 | 0.849 | 0.869 | 0.891 | 0.828 | 0.953 | 0.917 | 0.846 | 0.989 |
| OAS1_IL1R2_OLAH_RETN_MMP8 | 0.859 | 0.849 | 0.869 | 0.893 | 0.826 | 0.960 | 0.949 | 0.900 | 0.997 |
| RSAD2_IFIT1_SIGLEC1_SLC1A2_RETN | 0.859 | 0.850 | 0.868 | 0.895 | 0.832 | 0.957 | 0.945 | 0.893 | 0.996 |
| RSAD2_IFI44L_OLAH_FAM20A_MMP8 | 0.859 | 0.848 | 0.869 | 0.888 | 0.817 | 0.959 | 0.957 | 0.909 | 1 |
| OAS1_ISG15_HERC6_OLAH_MMP8 | 0.859 | 0.849 | 0.869 | 0.888 | 0.822 | 0.954 | 0.949 | 0.902 | 0.996 |
| RSAD2_IFIT1_ISG15_IL1R2_MMP8 | 0.859 | 0.850 | 0.868 | 0.892 | 0.829 | 0.954 | 0.960 | 0.920 | 1.000 |
| IFIT1_ISG15_IL1R2_RETN_MMP8 | 0.859 | 0.849 | 0.869 | 0.892 | 0.825 | 0.959 | 0.967 | 0.933 | 1 |
| OAS1_IFIT1_IFI44L_HERC6_OLAH | 0.859 | 0.849 | 0.869 | 0.887 | 0.821 | 0.953 | 0.941 | 0.889 | 0.994 |
| IFI44L_SIGLEC1_ISG15_SLC1A2_RETN | 0.859 | 0.850 | 0.868 | 0.894 | 0.830 | 0.957 | 0.951 | 0.903 | 0.999 |
| IFIT1_IFI44L_ISG15_HERC6_OLAH | 0.859 | 0.849 | 0.868 | 0.886 | 0.820 | 0.952 | 0.945 | 0.894 | 0.995 |
| OAS1_IFI44L_HERC6_OLAH_MMP8 | 0.859 | 0.849 | 0.869 | 0.888 | 0.821 | 0.954 | 0.941 | 0.889 | 0.993 |
| IFIT1_IFI44L_ISG15_IL1R2_RETN | 0.859 | 0.849 | 0.868 | 0.888 | 0.821 | 0.956 | 0.957 | 0.914 | 0.999 |
| IFIT1_ISG15_OLAH_FAM20A_RETN | 0.859 | 0.848 | 0.869 | 0.887 | 0.815 | 0.960 | 0.958 | 0.910 | 1 |
| OAS1_IFI44L_ISG15_SLC1A2_IL1R2 | 0.859 | 0.850 | 0.868 | 0.899 | 0.843 | 0.955 | 0.939 | 0.883 | 0.995 |
| ISG15_HERC6_SLC1A2_FAM20A_MMP8 | 0.859 | 0.848 | 0.869 | 0.890 | 0.826 | 0.955 | 0.925 | 0.840 | 1 |
| RSAD2_IFIT1_IFI44L_ISG15_IL1R2 | 0.859 | 0.849 | 0.868 | 0.890 | 0.823 | 0.956 | 0.958 | 0.916 | 0.999 |
| RSAD2_ISG15_HERC6_OLAH_MMP8 | 0.859 | 0.849 | 0.868 | 0.889 | 0.824 | 0.953 | 0.945 | 0.895 | 0.994 |
| OAS1_IFI44L_ISG15_IL1R2_OLAH | 0.859 | 0.849 | 0.868 | 0.893 | 0.826 | 0.959 | 0.942 | 0.890 | 0.995 |
| OAS1_IFI44L_IL1R2_OLAH_RETN | 0.849 | 0.868 | 0.893 | 0.893 | 0.824 | 0.961 | 0.946 | 0.895 | 0.996 |
| IFIT1_IFI44L_OLAH_FAM20A_RETN | 0.859 | 0.848 | 0.869 | 0.888 | 0.816 | 0.959 | 0.957 | 0.909 | 1 |
| IFI27_IFIT1_IFI44L_SIGLEC1_HERC6 | 0.859 | 0.849 | 0.868 | 0.893 | 0.834 | 0.952 | 0.920 | 0.856 | 0.984 |
| RSAD2_ISG15_OLAH_FAM20A_MMP8 | 0.859 | 0.848 | 0.869 | 0.887 | 0.815 | 0.959 | 0.960 | 0.910 | 1 |
| RSAD2_OAS1_IFIT1_HERC6_OLAH | 0.859 | 0.849 | 0.868 | 0.886 | 0.819 | 0.953 | 0.937 | 0.882 | 0.992 |
| OAS1_IFIT1_ISG15_HERC6_OLAH | 0.858 | 0.848 | 0.868 | 0.885 | 0.819 | 0.950 | 0.951 | 0.905 | 0.997 |
| IFI44L_ISG15_IL1R2_OLAH_RETN | 0.858 | 0.849 | 0.868 | 0.891 | 0.826 | 0.957 | 0.954 | 0.909 | 1.000 |
| IFI44L_SIGLEC1_HERC6_SLC1A2_MMP8 | 0.858 | 0.849 | 0.867 | 0.886 | 0.821 | 0.951 | 0.936 | 0.879 | 0.993 |
| RSAD2_OLAH_FAM20A_RETN_MMP8 | 0.858 | 0.848 | 0.869 | 0.888 | 0.817 | 0.960 | 0.962 | 0.913 | 1 |
| RSAD2_OAS1_IFI44L_SLC1A2_IL1R2 | 0.858 | 0.849 | 0.867 | 0.897 | 0.839 | 0.954 | 0.940 | 0.883 | 0.998 |
| RSAD2_IFIT1_ISG15_HERC6_RETN | 0.858 | 0.848 | 0.868 | 0.890 | 0.821 | 0.958 | 0.937 | 0.881 | 0.993 |
| ISG15_SLC1A2_OLAH_RETN_MMP8 | 0.858 | 0.849 | 0.868 | 0.898 | 0.839 | 0.957 | 0.935 | 0.873 | 0.996 |
| RSAD2_OAS1_IFI44L_HERC6_OLAH | 0.858 | 0.848 | 0.868 | 0.887 | 0.821 | 0.953 | 0.937 | 0.882 | 0.992 |
| OAS1_ISG15_HERC6_RETN_MMP8 | 0.858 | 0.848 | 0.868 | 0.890 | 0.822 | 0.958 | 0.941 | 0.886 | 0.996 |
| RSAD2_IFI44L_SLC1A2_IL1R2_RETN | 0.858 | 0.849 | 0.867 | 0.897 | 0.838 | 0.955 | 0.941 | 0.884 | 0.999 |
| IFIT1_ISG15_HERC6_FAM20A_MMP8 | 0.858 | 0.848 | 0.868 | 0.885 | 0.817 | 0.952 | 0.927 | 0.845 | 1 |
| OAS1_ISG15_IL1R2_OLAH_RETN | 0.858 | 0.848 | 0.868 | 0.894 | 0.826 | 0.962 | 0.942 | 0.889 | 0.996 |
| RSAD2_IFI27_ISG15_SLC1A2_MMP8 | 0.858 | 0.848 | 0.867 | 0.888 | 0.824 | 0.952 | 0.911 | 0.833 | 0.989 |
| RSAD2_OAS1_ISG15_SLC1A2_IL1R2 | 0.858 | 0.849 | 0.867 | 0.895 | 0.837 | 0.953 | 0.938 | 0.875 | 1 |
| RSAD2_IFIT1_ISG15_IL1R2_OLAH | 0.858 | 0.848 | 0.867 | 0.893 | 0.827 | 0.958 | 0.952 | 0.907 | 0.997 |
| RSAD2_IFI44L_SLC1A2_IL1R2_MMP8 | 0.858 | 0.849 | 0.866 | 0.896 | 0.838 | 0.953 | 0.945 | 0.887 | 1 |
| IFI44L_IL1R2_OLAH_RETN_MMP8 | 0.858 | 0.848 | 0.868 | 0.889 | 0.824 | 0.954 | 0.957 | 0.913 | 1.000 |
| IFI27_IFIT1_SIGLEC1_ISG15_HERC6 | 0.858 | 0.848 | 0.868 | 0.889 | 0.830 | 0.949 | 0.924 | 0.861 | 0.987 |
| IFIT1_ISG15_OLAH_FAM20A_MMP8 | 0.858 | 0.847 | 0.868 | 0.884 | 0.811 | 0.956 | 0.959 | 0.910 | 1 |
| RSAD2_IL1R2_OLAH_RETN_MMP8 | 0.858 | 0.848 | 0.868 | 0.890 | 0.823 | 0.957 | 0.964 | 0.927 | 1 |
| RSAD2_ISG15_SLC1A2_IL1R2_MMP8 | 0.858 | 0.850 | 0.866 | 0.894 | 0.836 | 0.953 | 0.940 | 0.878 | 1 |
| OAS1_ISG15_SLC1A2_IL1R2_RETN | 0.858 | 0.849 | 0.867 | 0.899 | 0.841 | 0.956 | 0.930 | 0.867 | 0.994 |
| RSAD2_OAS1_ISG15_HERC6_FAM20A | 0.858 | 0.848 | 0.868 | 0.883 | 0.816 | 0.951 | 0.923 | 0.837 | 1 |
| RSAD2_SIGLEC1_ISG15_SLC1A2_RETN | 0.858 | 0.848 | 0.867 | 0.895 | 0.832 | 0.958 | 0.947 | 0.897 | 0.997 |
| RSAD2_IFIT1_IFI44L_OLAH_FAM20A | 0.858 | 0.848 | 0.868 | 0.887 | 0.815 | 0.959 | 0.955 | 0.906 | 1 |
| RSAD2_IFIT1_OLAH_FAM20A_MMP8 | 0.858 | 0.848 | 0.868 | 0.884 | 0.811 | 0.957 | 0.960 | 0.910 | 1 |
| OAS1_SIGLEC1_SLC1A2_RETN_MMP8 | 0.858 | 0.848 | 0.867 | 0.893 | 0.829 | 0.956 | 0.955 | 0.911 | 1 |
| RSAD2_IFIT1_ISG15_IL1R2_RETN | 0.858 | 0.848 | 0.867 | 0.886 | 0.817 | 0.956 | 0.957 | 0.914 | 0.999 |
| OAS1_IFIT1_SIGLEC1_SLC1A2_MMP8 | 0.858 | 0.848 | 0.867 | 0.886 | 0.823 | 0.950 | 0.928 | 0.869 | 0.988 |
| IFIT1_ISG15_IL1R2_OLAH_RETN | 0.858 | 0.848 | 0.867 | 0.892 | 0.826 | 0.958 | 0.962 | 0.923 | 1 |
| OAS1_IFI44L_SLC1A2_IL1R2_RETN | 0.858 | 0.848 | 0.867 | 0.898 | 0.840 | 0.956 | 0.937 | 0.880 | 0.993 |
| RSAD2_ISG15_HERC6_FAM20A_MMP8 | 0.858 | 0.847 | 0.868 | 0.888 | 0.822 | 0.954 | 0.925 | 0.843 | 1 |
| OAS1_SIGLEC1_ISG15_HERC6_SLC1A2 | 0.858 | 0.848 | 0.867 | 0.885 | 0.823 | 0.948 | 0.947 | 0.897 | 0.996 |
| RSAD2_IFI44L_HERC6_OLAH_MMP8 | 0.858 | 0.848 | 0.867 | 0.888 | 0.822 | 0.954 | 0.937 | 0.882 | 0.992 |
| RSAD2_IFIT1_HERC6_OLAH_MMP8 | 0.858 | 0.848 | 0.867 | 0.887 | 0.820 | 0.954 | 0.941 | 0.889 | 0.994 |
| RSAD2_ISG15_IL1R2_OLAH_MMP8 | 0.857 | 0.848 | 0.867 | 0.890 | 0.826 | 0.953 | 0.958 | 0.916 | 1.000 |
| RSAD2_HERC6_FAM20A_RETN_MMP8 | 0.857 | 0.847 | 0.868 | 0.888 | 0.818 | 0.959 | 0.940 | 0.863 | 1 |
| RSAD2_ISG15_HERC6_SLC1A2_FAM20A | 0.857 | 0.847 | 0.867 | 0.887 | 0.825 | 0.950 | 0.929 | 0.846 | 1 |
| IFI44L_HERC6_SLC1A2_FAM20A_MMP8 | 0.857 | 0.847 | 0.868 | 0.886 | 0.821 | 0.952 | 0.928 | 0.845 | 1 |
| IFIT1_IFI44L_HERC6_FAM20A_MMP8 | 0.857 | 0.847 | 0.868 | 0.885 | 0.816 | 0.953 | 0.922 | 0.840 | 1 |
| RSAD2_OAS1_SLC1A2_IL1R2_RETN | 0.857 | 0.848 | 0.867 | 0.896 | 0.836 | 0.955 | 0.937 | 0.874 | 1 |
| OAS1_IFIT1_SIGLEC1_HERC6_MMP8 | 0.857 | 0.848 | 0.867 | 0.882 | 0.815 | 0.949 | 0.888 | 0.807 | 0.969 |
| RSAD2_IFIT1_SIGLEC1_HERC6_SLC1A2 | 0.857 | 0.848 | 0.866 | 0.890 | 0.829 | 0.951 | 0.925 | 0.865 | 0.985 |
| IFI27_OAS1_ISG15_SLC1A2_MMP8 | 0.857 | 0.848 | 0.867 | 0.887 | 0.823 | 0.951 | 0.910 | 0.832 | 0.988 |
| OAS1_HERC6_FAM20A_RETN_MMP8 | 0.857 | 0.847 | 0.868 | 0.889 | 0.818 | 0.960 | 0.939 | 0.861 | 1 |
| RSAD2_IFIT1_IL1R2_RETN_MMP8 | 0.857 | 0.847 | 0.867 | 0.890 | 0.821 | 0.959 | 0.965 | 0.929 | 1 |
| RSAD2_ISG15_OLAH_FAM20A_RETN | 0.857 | 0.847 | 0.867 | 0.886 | 0.813 | 0.960 | 0.960 | 0.908 | 1 |
| RSAD2_IFI44L_IL1R2_OLAH_MMP8 | 0.857 | 0.848 | 0.867 | 0.890 | 0.827 | 0.952 | 0.953 | 0.908 | 0.999 |
| RSAD2_OAS1_IFIT1_SIGLEC1_RETN | 0.857 | 0.847 | 0.867 | 0.880 | 0.805 | 0.954 | 0.941 | 0.887 | 0.996 |
| IFIT1_SIGLEC1_SLC1A2_RETN_MMP8 | 0.857 | 0.848 | 0.866 | 0.895 | 0.833 | 0.957 | 0.950 | 0.903 | 0.997 |
| OAS1_IFI44L_SLC1A2_IL1R2_MMP8 | 0.857 | 0.848 | 0.866 | 0.897 | 0.840 | 0.954 | 0.934 | 0.874 | 0.993 |
| RSAD2_IFIT1_IFI44L_IL1R2_OLAH | 0.857 | 0.847 | 0.867 | 0.891 | 0.825 | 0.957 | 0.952 | 0.907 | 0.997 |
| RSAD2_IFIT1_ISG15_OLAH_FAM20A | 0.857 | 0.847 | 0.867 | 0.885 | 0.810 | 0.959 | 0.960 | 0.911 | 1 |
| RSAD2_IFI44L_SIGLEC1_SLC1A2_RETN | 0.857 | 0.848 | 0.866 | 0.894 | 0.831 | 0.958 | 0.950 | 0.902 | 0.998 |
| RSAD2_IFI27_IFIT1_SLC1A2_MMP8 | 0.857 | 0.847 | 0.867 | 0.888 | 0.824 | 0.952 | 0.920 | 0.847 | 0.992 |
| OAS1_IFI44L_ISG15_HERC6_OLAH | 0.857 | 0.847 | 0.867 | 0.885 | 0.820 | 0.951 | 0.950 | 0.904 | 0.996 |
| RSAD2_OAS1_SIGLEC1_HERC6_SLC1A2 | 0.857 | 0.848 | 0.866 | 0.887 | 0.825 | 0.949 | 0.938 | 0.885 | 0.991 |
| IFIT1_IFI44L_SIGLEC1_HERC6_MMP8 | 0.857 | 0.847 | 0.866 | 0.882 | 0.814 | 0.950 | 0.886 | 0.803 | 0.968 |
| IFI44L_ISG15_IL1R2_RETN_MMP8 | 0.857 | 0.847 | 0.866 | 0.890 | 0.827 | 0.954 | 0.966 | 0.931 | 1 |
| RSAD2_IFIT1_IFI44L_HERC6_OLAH | 0.857 | 0.847 | 0.866 | 0.888 | 0.823 | 0.953 | 0.938 | 0.884 | 0.992 |
| RSAD2_OAS1_SLC1A2_IL1R2_MMP8 | 0.857 | 0.848 | 0.866 | 0.896 | 0.837 | 0.954 | 0.939 | 0.878 | 1.000 |
| RSAD2_IFI44L_ISG15_HERC6_OLAH | 0.857 | 0.847 | 0.867 | 0.888 | 0.824 | 0.952 | 0.942 | 0.892 | 0.993 |
| IFIT1_IFI44L_ISG15_HERC6_FAM20A | 0.857 | 0.847 | 0.867 | 0.880 | 0.810 | 0.950 | 0.928 | 0.843 | 1 |
| OAS1_IFI44L_HERC6_SLC1A2_RETN | 0.857 | 0.847 | 0.867 | 0.888 | 0.821 | 0.956 | 0.936 | 0.874 | 0.997 |
| RSAD2_ISG15_HERC6_SLC1A2_RETN | 0.857 | 0.847 | 0.866 | 0.892 | 0.827 | 0.956 | 0.935 | 0.871 | 0.998 |
| RSAD2_SLC1A2_IL1R2_RETN_MMP8 | 0.857 | 0.848 | 0.866 | 0.895 | 0.836 | 0.955 | 0.941 | 0.880 | 1 |
| RSAD2_ISG15_HERC6_RETN_MMP8 | 0.857 | 0.846 | 0.867 | 0.890 | 0.822 | 0.958 | 0.936 | 0.879 | 0.992 |
| RSAD2_IFI44L_ISG15_IL1R2_OLAH | 0.857 | 0.847 | 0.866 | 0.886 | 0.824 | 0.955 | 0.952 | 0.906 | 0.999 |
| RSAD2_IFIT1_IFI44L_IL1R2_RETN | 0.857 | 0.847 | 0.866 | 0.885 | 0.814 | 0.956 | 0.955 | 0.913 | 0.998 |
| OAS1_IFIT1_SIGLEC1_ISG15_RETN | 0.857 | 0.847 | 0.866 | 0.880 | 0.806 | 0.954 | 0.945 | 0.892 | 0.997 |
| RSAD2_ISG15_IL1R2_OLAH_RETN | 0.856 | 0.847 | 0.866 | 0.889 | 0.822 | 0.957 | 0.958 | 0.915 | 1 |
| RSAD2_IFIT1_ISG15_HERC6_OLAH | 0.856 | 0.847 | 0.866 | 0.886 | 0.821 | 0.951 | 0.945 | 0.895 | 0.994 |
| RSAD2_IFIT1_IL1R2_OLAH_RETN | 0.856 | 0.847 | 0.866 | 0.889 | 0.821 | 0.957 | 0.953 | 0.909 | 0.998 |
| OAS1_ISG15_HERC6_SLC1A2_FAM20A | 0.856 | 0.846 | 0.866 | 0.888 | 0.825 | 0.950 | 0.930 | 0.847 | 1 |
| OAS1_ISG15_SLC1A2_IL1R2_MMP8 | 0.856 | 0.848 | 0.865 | 0.898 | 0.840 | 0.955 | 0.925 | 0.858 | 0.992 |
| OAS1_IFI44L_SIGLEC1_HERC6_SLC1A2 | 0.856 | 0.847 | 0.865 | 0.887 | 0.824 | 0.950 | 0.945 | 0.894 | 0.995 |
| IFI44L_SIGLEC1_SLC1A2_RETN_MMP8 | 0.856 | 0.847 | 0.865 | 0.892 | 0.828 | 0.957 | 0.953 | 0.907 | 0.999 |
| RSAD2_SIGLEC1_SLC1A2_RETN_MMP8 | 0.856 | 0.846 | 0.866 | 0.893 | 0.829 | 0.957 | 0.953 | 0.908 | 0.999 |
| SIGLEC1_ISG15_SLC1A2_RETN_MMP8 | 0.856 | 0.847 | 0.866 | 0.893 | 0.829 | 0.958 | 0.953 | 0.906 | 1 |
| RSAD2_IFI44L_OLAH_FAM20A_RETN | 0.856 | 0.846 | 0.867 | 0.888 | 0.816 | 0.960 | 0.954 | 0.903 | 1 |
| SIGLEC1_ISG15_HERC6_SLC1A2_MMP8 | 0.856 | 0.847 | 0.865 | 0.887 | 0.822 | 0.952 | 0.934 | 0.877 | 0.991 |
| IFI44L_ISG15_HERC6_SLC1A2_FAM20A | 0.856 | 0.846 | 0.866 | 0.886 | 0.821 | 0.952 | 0.935 | 0.854 | 1 |
| RSAD2_OAS1_IFI44L_HERC6_FAM20A | 0.856 | 0.846 | 0.866 | 0.882 | 0.811 | 0.952 | 0.923 | 0.841 | 1 |
| RSAD2_IFIT1_SIGLEC1_SLC1A2_MMP8 | 0.856 | 0.847 | 0.865 | 0.886 | 0.822 | 0.949 | 0.928 | 0.869 | 0.988 |
| OAS1_IFIT1_IFI44L_SIGLEC1_RETN | 0.856 | 0.845 | 0.866 | 0.876 | 0.800 | 0.951 | 0.935 | 0.877 | 0.993 |
| ISG15_SLC1A2_IL1R2_RETN_MMP8 | 0.856 | 0.847 | 0.865 | 0.897 | 0.840 | 0.954 | 0.918 | 0.838 | 0.999 |
| IFIT1_IFI44L_SIGLEC1_SLC1A2_MMP8 | 0.856 | 0.847 | 0.865 | 0.888 | 0.825 | 0.951 | 0.921 | 0.858 | 0.984 |
| IFIT1_IFI44L_HERC6_SLC1A2_FAM20A | 0.856 | 0.846 | 0.865 | 0.892 | 0.831 | 0.953 | 0.926 | 0.842 | 1 |
| IFIT1_OLAH_FAM20A_RETN_MMP8 | 0.856 | 0.845 | 0.866 | 0.887 | 0.816 | 0.958 | 0.964 | 0.921 | 1 |
| RSAD2_IFI44L_IL1R2_OLAH_RETN | 0.856 | 0.846 | 0.865 | 0.890 | 0.822 | 0.957 | 0.957 | 0.913 | 1.000 |
| RSAD2_OAS1_IFI44L_ISG15_IL1R2 | 0.856 | 0.846 | 0.865 | 0.888 | 0.823 | 0.952 | 0.941 | 0.889 | 0.993 |
| IFIT1_IFI44L_SIGLEC1_ISG15_RETN | 0.856 | 0.846 | 0.865 | 0.881 | 0.808 | 0.954 | 0.939 | 0.884 | 0.995 |
| IFIT1_IFI44L_IL1R2_OLAH_RETN | 0.855 | 0.846 | 0.865 | 0.890 | 0.823 | 0.957 | 0.954 | 0.910 | 0.999 |
| RSAD2_SIGLEC1_ISG15_HERC6_SLC1A2 | 0.855 | 0.847 | 0.864 | 0.887 | 0.825 | 0.949 | 0.938 | 0.884 | 0.992 |
| RSAD2_OAS1_IFI44L_IL1R2_RETN | 0.855 | 0.846 | 0.865 | 0.883 | 0.813 | 0.953 | 0.942 | 0.891 | 0.994 |
| OAS1_IFI44L_ISG15_IL1R2_RETN | 0.855 | 0.846 | 0.865 | 0.888 | 0.820 | 0.956 | 0.952 | 0.908 | 0.997 |
| RSAD2_IFIT1_OLAH_FAM20A_RETN | 0.855 | 0.845 | 0.866 | 0.885 | 0.811 | 0.958 | 0.958 | 0.907 | 1 |
| RSAD2_IFIT1_SIGLEC1_HERC6_MMP8 | 0.855 | 0.846 | 0.865 | 0.881 | 0.813 | 0.949 | 0.887 | 0.805 | 0.969 |
| RSAD2_OAS1_ISG15_IL1R2_RETN | 0.855 | 0.846 | 0.865 | 0.886 | 0.817 | 0.954 | 0.947 | 0.898 | 0.995 |
| RSAD2_OAS1_IFI44L_IL1R2_MMP8 | 0.855 | 0.846 | 0.865 | 0.887 | 0.822 | 0.951 | 0.943 | 0.893 | 0.994 |
| IFIT1_HERC6_SLC1A2_FAM20A_MMP8 | 0.855 | 0.845 | 0.865 | 0.886 | 0.820 | 0.951 | 0.925 | 0.840 | 1 |
| RSAD2_IFI44L_SIGLEC1_HERC6_SLC1A2 | 0.855 | 0.846 | 0.864 | 0.886 | 0.824 | 0.948 | 0.934 | 0.878 | 0.989 |
| IFI44L_SIGLEC1_ISG15_SLC1A2_MMP8 | 0.855 | 0.847 | 0.864 | 0.884 | 0.819 | 0.949 | 0.937 | 0.882 | 0.991 |
| OAS1_SLC1A2_IL1R2_RETN_MMP8 | 0.855 | 0.846 | 0.864 | 0.895 | 0.836 | 0.955 | 0.928 | 0.863 | 0.994 |
| RSAD2_HERC6_SLC1A2_FAM20A_MMP8 | 0.855 | 0.845 | 0.865 | 0.888 | 0.823 | 0.952 | 0.924 | 0.839 | 1 |
| ISG15_IL1R2_OLAH_RETN_MMP8 | 0.855 | 0.846 | 0.865 | 0.892 | 0.830 | 0.954 | 0.943 | 0.886 | 1 |
| IFI44L_SLC1A2_FAM20A_RETN_MMP8 | 0.855 | 0.845 | 0.865 | 0.891 | 0.825 | 0.957 | 0.930 | 0.852 | 1 |
| IFIT1_SIGLEC1_ISG15_SLC1A2_MMP8 | 0.855 | 0.846 | 0.864 | 0.889 | 0.827 | 0.952 | 0.930 | 0.872 | 0.989 |
| RSAD2_OAS1_IFITI_OLAH_RETN | 0.855 | 0.845 | 0.865 | 0.884 | 0.809 | 0.958 | 0.947 | 0.896 | 0.998 |
| IFI44L_ISG15_SLC1A2_FAM20A_MMP8 | 0.855 | 0.845 | 0.865 | 0.884 | 0.815 | 0.953 | 0.934 | 0.858 | 1 |
| OAS1_ISG15_HERC6_FAM20A_MMP8 | 0.855 | 0.844 | 0.865 | 0.885 | 0.818 | 0.953 | 0.932 | 0.851 | 1 |
| OAS1_IFIT1_SIGLEC1_RETN_MMP8 | 0.855 | 0.844 | 0.865 | 0.878 | 0.803 | 0.954 | 0.946 | 0.894 | 0.997 |
| RSAD2_IFIT1_{——}IFI44L_SIGLEC1_RETN | 0.855 | 0.844 | 0.865 | 0.878 | 0.803 | 0.953 | 0.936 | 0.878 | 0.994 |
| RSAD2_OAS1_IL1R2_RETN_MMP8 | 0.855 | 0.845 | 0.864 | 0.885 | 0.815 | 0.954 | 0.953 | 0.909 | 0.997 |
| RSAD2_IFI44L_ISG15_IL1R2_RETN | 0.855 | 0.845 | 0.864 | 0.884 | 0.815 | 0.953 | 0.958 | 0.916 | 0.999 |
| RSAD2_IFIT1_SIGLEC1_ISG15_RETN | 0.854 | 0.845 | 0.864 | 0.880 | 0.806 | 0.954 | 0.943 | 0.890 | 0.997 |
| OAS1_IFI44L_HERC6_SLC1A2_FAM20A | 0.854 | 0.844 | 0.865 | 0.889 | 0.827 | 0.952 | 0.935 | 0.854 | 1 |
| RSAD2_OAS1_SIGLEC1_SLC1A2_MMP8 | 0.854 | 0.845 | 0.864 | 0.883 | 0.818 | 0.948 | 0.938 | 0.883 | 0.993 |
| OAS1_IFI44L_ISG15_IL1R2_MMP8 | 0.854 | 0.845 | 0.863 | 0.887 | 0.823 | 0.950 | 0.946 | 0.897 | 0.995 |
| ISG15_OLAH_FAM20A_RETN_MMP8 | 0.854 | 0.844 | 0.865 | 0.891 | 0.826 | 0.956 | 0.950 | 0.899 | 1 |
| IFIT1_ISG15_HERC6_SLC1A2_FAM20A | 0.854 | 0.844 | 0.865 | 0.886 | 0.822 | 0.950 | 0.933 | 0.850 | 1 |
| OAS1_HERC6_SLC1A2_FAM20A_MMP8 | 0.854 | 0.844 | 0.864 | 0.887 | 0.822 | 0.952 | 0.921 | 0.835 | 1 |
| RSAD2_OAS1_OLAH_RETN_MMP8 | 0.854 | 0.844 | 0.865 | 0.883 | 0.809 | 0.958 | 0.959 | 0.917 | 1 |
| OAS1_ISG15_IL1R2_RETN_MMP8 | 0.854 | 0.845 | 0.864 | 0.889 | 0.823 | 0.955 | 0.953 | 0.909 | 0.997 |
| IFI27_OAS1_IFIT1_SIGLEC1_ISG15 | 0.854 | 0.845 | 0.864 | 0.882 | 0.818 | 0.946 | 0.941 | 0.887 | 0.995 |
| OAS1_IFIT1_ISG15_OLAH_RETN | 0.854 | 0.844 | 0.864 | 0.884 | 0.810 | 0.959 | 0.955 | 0.911 | 1.000 |
| RSAD2_OAS1_ISG15_IL1R2_MMP8 | 0.854 | 0.845 | 0.863 | 0.886 | 0.821 | 0.951 | 0.945 | 0.895 | 0.994 |
| IFIT1_IFI44L_SIGLEC1_HERC6_SLC1A2 | 0.854 | 0.846 | 0.863 | 0.886 | 0.825 | 0.948 | 0.934 | 0.878 | 0.990 |
| SLC1A2_IL1R2_OLAH_FAM20A_MMP8 | 0.854 | 0.845 | 0.864 | 0.891 | 0.835 | 0.947 | 0.877 | 0.782 | 0.973 |
| RSAD2_IFI44L_HERC6_SLC1A2_RETN | 0.854 | 0.844 | 0.864 | 0.890 | 0.823 | 0.957 | 0.929 | 0.867 | 0.992 |
| OAS1_IFI44L_SIGLEC1_SLC1A2_MMP8 | 0.854 | 0.845 | 0.863 | 0.884 | 0.819 | 0.949 | 0.937 | 0.882 | 0.992 |
| OAS1_IFIT1_OLAH_RETN_MMP8 | 0.854 | 0.844 | 0.864 | 0.882 | 0.807 | 0.957 | 0.963 | 0.925 | 1 |
| RSAD2_OAS1_IFI44L_HERC6_RETN | 0.854 | 0.843 | 0.864 | 0.877 | 0.801 | 0.954 | 0.929 | 0.870 | 0.988 |
| IFI44L_ISG15_HERC6_FAM20A_MMP8 | 0.854 | 0.843 | 0.865 | 0.878 | 0.806 | 0.950 | 0.928 | 0.847 | 1 |
| IFIT1_IFI44L_ISG15_HERC6_RETN | 0.854 | 0.844 | 0.864 | 0.884 | 0.812 | 0.956 | 0.935 | 0.877 | 0.992 |
| IFIT1_SIGLEC1_ISG15_HERC6_SLC1A2 | 0.854 | 0.845 | 0.863 | 0.886 | 0.825 | 0.948 | 0.936 | 0.881 | 0.991 |
| RSAD2_IFI27_OAS1_SIGLEC1_ISG15 | 0.854 | 0.844 | 0.864 | 0.884 | 0.822 | 0.946 | 0.943 | 0.889 | 0.998 |
| OAS1_SIGLEC1_ISG15_SLC1A2_MMP8 | 0.854 | 0.844 | 0.863 | 0.885 | 0.821 | 0.949 | 0.939 | 0.885 | 0.993 |
| RSAD2_IFIT1_ISG15_HERC6_FAM20A | 0.854 | 0.844 | 0.864 | 0.883 | 0.816 | 0.950 | 0.925 | 0.840 | 1 |
| RSAD2_IFI27_OAS1_IFI44L_SIGLEC1 | 0.854 | 0.844 | 0.864 | 0.885 | 0.823 | 0.947 | 0.946 | 0.894 | 0.997 |
| IFIT1_ISG15_SLC1A2_FAM20A_RETN | 0.854 | 0.843 | 0.864 | 0.885 | 0.816 | 0.955 | 0.936 | 0.862 | 1 |
| IFI44L_ISG15_OLAH_RETN_MMP8 | 0.854 | 0.844 | 0.864 | 0.885 | 0.813 | 0.956 | 0.959 | 0.916 | 1 |
| OAS1_IFI44L_IL1R2_RETN_MMP8 | 0.854 | 0.844 | 0.863 | 0.886 | 0.818 | 0.955 | 0.964 | 0.927 | 1 |
| IFI27_OAS1_IFIT1_ISG15_MMP8 | 0.853 | 0.843 | 0.863 | 0.878 | 0.807 | 0.948 | 0.914 | 0.842 | 0.986 |
| IFI27_OAS1_IFIT1_HERC6_SLC1A2 | 0.853 | 0.843 | 0.863 | 0.888 | 0.827 | 0.949 | 0.940 | 0.881 | 1.000 |
| IFIT1_ISG15_HERC6_SLC1A2_RETN | 0.853 | 0.844 | 0.863 | 0.888 | 0.821 | 0.954 | 0.940 | 0.879 | 1 |
| IFI27_OAS1_IFI44L_SIGLEC1_ISG15 | 0.853 | 0.844 | 0.863 | 0.886 | 0.823 | 0.948 | 0.948 | 0.897 | 0.999 |
| IFI27_OAS1_IFIT1_IFI44L_SIGLEC1 | 0.853 | 0.843 | 0.863 | 0.884 | 0.821 | 0.948 | 0.951 | 0.902 | 1 |
| RSAD2_OAS1_IFIT1_OLAH_MMP8 | 0.853 | 0.843 | 0.863 | 0.880 | 0.807 | 0.953 | 0.946 | 0.895 | 0.997 |
| OAS1_IFIT1_IFI44L_OLAH_RETN | 0.853 | 0.843 | 0.864 | 0.882 | 0.807 | 0.956 | 0.946 | 0.895 | 0.996 |
| RSAD2_OAS1_ISG15_OLAH_RETN | 0.853 | 0.843 | 0.863 | 0.883 | 0.809 | 0.957 | 0.953 | 0.907 | 0.999 |
| RSAD2_IFI27_IFI44L_SIGLEC1_ISG15 | 0.853 | 0.843 | 0.863 | 0.886 | 0.825 | 0.948 | 0.942 | 0.888 | 0.996 |
| IFI27_OAS1_IFI44L_ISG15_MMP8 | 0.853 | 0.843 | 0.863 | 0.880 | 0.810 | 0.949 | 0.904 | 0.827 | 0.981 |
| RSAD2_ISG15_IL1R2_RETN_MMP8 | 0.853 | 0.844 | 0.863 | 0.886 | 0.818 | 0.954 | 0.962 | 0.922 | 1 |
| RSAD2_OAS1_SIGLEC1_ISG15_RETN | 0.853 | 0.843 | 0.863 | 0.878 | 0.803 | 0.953 | 0.949 | 0.899 | 0.999 |
| RSAD2_IFI27_OAS1_IFIT1_SIGLEC1 | 0.853 | 0.843 | 0.863 | 0.884 | 0.822 | 0.947 | 0.942 | 0.888 | 0.997 |
| RSAD2_IFI27_OAS1_IFI44L_MMP8 | 0.853 | 0.843 | 0.863 | 0.877 | 0.807 | 0.948 | 0.909 | 0.833 | 0.985 |
| IFI27_OAS1_IFI44L_HERC6_SLC1A2 | 0.853 | 0.843 | 0.863 | 0.884 | 0.823 | 0.945 | 0.921 | 0.853 | 0.989 |
| OAS1_IFIT1_IFI44L_HERC6_RETN | 0.853 | 0.842 | 0.864 | 0.876 | 0.799 | 0.953 | 0.929 | 0.870 | 0.988 |
| IFI27_IFIT1_IFI44L_ISG15_MMP8 | 0.853 | 0.843 | 0.863 | 0.878 | 0.808 | 0.949 | 0.911 | 0.837 | 0.985 |
| IFIT1_IFI44L_SIGLEC1_RETN_MMP8 | 0.853 | 0.842 | 0.863 | 0.878 | 0.804 | 0.953 | 0.939 | 0.882 | 0.996 |
| IFI27_IFIT1_IFI44L_SIGLEC1_ISG15 | 0.853 | 0.843 | 0.863 | 0.885 | 0.821 | 0.949 | 0.941 | 0.887 | 0.996 |
| RSAD2_IFI44L_ISG15_SLC1A2_FAM20A | 0.853 | 0.842 | 0.863 | 0.888 | 0.821 | 0.955 | 0.924 | 0.845 | 1 |
| RSAD2_OAS1_HERC6_SLC1A2_FAM20A | 0.853 | 0.843 | 0.863 | 0.881 | 0.816 | 0.947 | 0.923 | 0.837 | 1 |
| RSAD2_IFI27_IFIT1_SIGLEC1_ISG15 | 0.853 | 0.843 | 0.863 | 0.883 | 0.820 | 0.946 | 0.942 | 0.888 | 0.997 |
| OAS1_IFI44L_OLAH_RETN_MMP8 | 0.853 | 0.842 | 0.863 | 0.882 | 0.807 | 0.957 | 0.959 | 0.918 | 1.000 |
| IFIT1_ISG15_HERC6_RETN_MMP8 | 0.853 | 0.842 | 0.863 | 0.884 | 0.813 | 0.955 | 0.927 | 0.866 | 0.989 |
| ISG15_HERC6_SLC1A2_RETN_MMP8 | 0.853 | 0.843 | 0.863 | 0.886 | 0.818 | 0.954 | 0.935 | 0.870 | 0.999 |
| RSAD2_SIGLEC1_ISG15_SLC1A2_MMP8 | 0.853 | 0.843 | 0.862 | 0.885 | 0.820 | 0.949 | 0.938 | 0.884 | 0.992 |
| RSAD2_IFI44L_ISG15_IL1R2_MMP8 | 0.853 | 0.844 | 0.862 | 0.886 | 0.823 | 0.949 | 0.961 | 0.921 | 1 |
| IFI44L_ISG15_HERC6_SLC1A2_RETN | 0.853 | 0.843 | 0.862 | 0.886 | 0.818 | 0.954 | 0.942 | 0.880 | 1 |
| OAS1_IFIT1_ISG15_HERC6_FAM20A | 0.852 | 0.842 | 0.863 | 0.879 | 0.810 | 0.948 | 0.926 | 0.839 | 1 |
| IFIT1_IFI44L_SLC1A2_FAM20A_RETN | 0.852 | 0.842 | 0.862 | 0.889 | 0.823 | 0.955 | 0.927 | 0.848 | 1 |
| OAS1_IFI44L_SLC1A2_FAM20A_RETN | 0.852 | 0.842 | 0.863 | 0.892 | 0.826 | 0.958 | 0.932 | 0.852 | 1 |
| SLC1A2_IL1R2_OLAH_FAM20A_RETN | 0.852 | 0.843 | 0.862 | 0.884 | 0.823 | 0.945 | 0.862 | 0.759 | 0.965 |
| RSAD2_IFIT1_IFI44L_HERC6_RETN | 0.852 | 0.842 | 0.863 | 0.876 | 0.799 | 0.953 | 0.927 | 0.867 | 0.987 |
| IFIT1_SIGLEC1_ISG15_RETN_MMP8 | 0.852 | 0.842 | 0.862 | 0.880 | 0.806 | 0.953 | 0.949 | 0.898 | 0.999 |
| RSAD2_OAS1_IFI44L_SLC1A2_FAM20A | 0.852 | 0.841 | 0.863 | 0.889 | 0.822 | 0.956 | 0.914 | 0.829 | 1.000 |
| IFI27_OAS1_ISG15_HERC6_SLC1A2 | 0.852 | 0.842 | 0.862 | 0.884 | 0.823 | 0.944 | 0.912 | 0.837 | 0.987 |
| RSAD2_IFI44L_HERC6_FAM20A_MMP8 | 0.852 | 0.841 | 0.863 | 0.879 | 0.808 | 0.950 | 0.922 | 0.840 | 1 |
| OAS1_IFI44L_SIGLEC1_ISG15_RETN | 0.852 | 0.842 | 0.862 | 0.877 | 0.801 | 0.953 | 0.952 | 0.904 | 1 |
| OAS1_ISG15_SLC1A2_FAM20A_RETN | 0.852 | 0.842 | 0.862 | 0.886 | 0.818 | 0.954 | 0.934 | 0.853 | 1 |
| OAS1_IFI44L_ISG15_HERC6_FAM20A | 0.852 | 0.842 | 0.862 | 0.878 | 0.807 | 0.948 | 0.933 | 0.853 | 1 |
| RSAD2_IFI44L_SIGLEC1_SLC1A2_MMP8 | 0.852 | 0.843 | 0.861 | 0.885 | 0.821 | 0.950 | 0.933 | 0.876 | 0.990 |
| RSAD2_OAS1_IFI44L_OLAH_MMP8 | 0.852 | 0.842 | 0.862 | 0.881 | 0.809 | 0.952 | 0.940 | 0.885 | 0.996 |
| RSAD2_OAS1_IFI44L_OLAH_RETN | 0.852 | 0.842 | 0.862 | 0.883 | 0.809 | 0.957 | 0.940 | 0.885 | 0.995 |
| RSAD2_IFI27_IFI44L_ISG15_MMP8 | 0.852 | 0.842 | 0.862 | 0.878 | 0.808 | 0.949 | 0.904 | 0.827 | 0.981 |
| RSAD2_IFI44L_IL1R2_RETN_MMP8 | 0.852 | 0.842 | 0.862 | 0.884 | 0.814 | 0.953 | 0.963 | 0.925 | 1 |
| RSAD2_OAS1_ISG15_FAM20A_RETN | 0.852 | 0.841 | 0.862 | 0.886 | 0.815 | 0.958 | 0.935 | 0.855 | 1 |
| OAS1_ISG15_OLAH_RETN_MMP8 | 0.852 | 0.841 | 0.862 | 0.883 | 0.810 | 0.957 | 0.963 | 0.925 | 1 |
| IFI44L_SIGLEC1_ISG15_RETN_MMP8 | 0.852 | 0.841 | 0.862 | 0.876 | 0.799 | 0.952 | 0.959 | 0.916 | 1 |
| OAS1_IFIT1_IFI44L_OLAH_MMP8 | 0.852 | 0.842 | 0.862 | 0.880 | 0.807 | 0.952 | 0.941 | 0.887 | 0.995 |
| RSAD2_IFI27_OAS1_ISG15_MMP8 | 0.852 | 0.841 | 0.862 | 0.877 | 0.807 | 0.948 | 0.908 | 0.831 | 0.985 |
| RSAD2_OAS1_IFI44L_SIGLEC1_RETN | 0.852 | 0.841 | 0.862 | 0.874 | 0.797 | 0.951 | 0.945 | 0.893 | 0.996 |
| RSAD2_OAS1_HERC6_FAM20A_MMP8 | 0.851 | 0.841 | 0.862 | 0.879 | 0.808 | 0.950 | 0.923 | 0.840 | 1 |
| RSAD2_IFI27_IFI44L_HERC6_SLC1A2 | 0.851 | 0.842 | 0.861 | 0.880 | 0.818 | 0.941 | 0.912 | 0.842 | 0.982 |
| RSAD2_OAS1_ISG15_OLAH_MMP8 | 0.851 | 0.842 | 0.861 | 0.880 | 0.808 | 0.952 | 0.948 | 0.897 | 0.999 |
| RSAD2_OAS1_IFIT1_ISG15_OLAH | 0.851 | 0.841 | 0.861 | 0.880 | 0.808 | 0.952 | 0.943 | 0.891 | 0.996 |
| IFI27_OAS1_IFIT1_IFI44L_MMP8 | 0.851 | 0.841 | 0.861 | 0.877 | 0.807 | 0.948 | 0.911 | 0.836 | 0.986 |
| RSAD2_IFIT1_SIGLEC1_RETN_MMP8 | 0.851 | 0.841 | 0.862 | 0.880 | 0.805 | 0.955 | 0.942 | 0.889 | 0.996 |
| OAS1_IFIT1_HERC6_FAM20A_MMP8 | 0.851 | 0.841 | 0.862 | 0.881 | 0.812 | 0.951 | 0.921 | 0.836 | 1 |
| RSAD2_IFI44L_SLC1A2_FAM20A_RETN | 0.851 | 0.841 | 0.862 | 0.889 | 0.823 | 0.955 | 0.927 | 0.848 | 1 |
| IFI27_OAS1_IFIT1_ISG15_HERC6 | 0.851 | 0.841 | 0.862 | 0.881 | 0.820 | 0.942 | 0.920 | 0.856 | 0.983 |
| OAS1_IFI44L_ISG15_OLAH_RETN | 0.851 | 0.841 | 0.861 | 0.882 | 0.808 | 0.957 | 0.955 | 0.912 | 0.999 |
| RSAD2_IFIT1_IFI44L_HERC6_FAM20A | 0.851 | 0.840 | 0.862 | 0.881 | 0.811 | 0.951 | 0.918 | 0.837 | 1 |
| OAS1_IFIT1_HERC6_SLC1A2_RETN | 0.851 | 0.841 | 0.861 | 0.882 | 0.812 | 0.952 | 0.938 | 0.873 | 1 |
| IFI44L_ISG15_HERC6_RETN_MMP8 | 0.851 | 0.840 | 0.861 | 0.880 | 0.804 | 0.955 | 0.941 | 0.889 | 0.994 |
| OAS1_IFIT1_ISG15_OLAH_MMP8 | 0.851 | 0.841 | 0.861 | 0.879 | 0.807 | 0.952 | 0.946 | 0.895 | 0.997 |
| RSAD2_IFI27_IFI44L_ISG15_HERC6 | 0.851 | 0.841 | 0.861 | 0.881 | 0.822 | 0.941 | 0.890 | 0.814 | 0.966 |
| RSAD2_OAS1_IFIT1_IFI44L_OLAH | 0.851 | 0.841 | 0.860 | 0.882 | 0.810 | 0.953 | 0.940 | 0.884 | 0.996 |
| RSAD2_IFI27_IFIT1_IFI44L_SIGLEC1 | 0.851 | 0.840 | 0.861 | 0.888 | 0.828 | 0.949 | 0.942 | 0.888 | 0.996 |
| IFI44L_SIGLEC1_ISG15_HERC6_MMP8 | 0.851 | 0.841 | 0.861 | 0.875 | 0.802 | 0.948 | 0.916 | 0.850 | 0.982 |
| IFI44L_SIGLEC1_ISG15_HERC6_SLC1A2 | 0.850 | 0.842 | 0.859 | 0.881 | 0.815 | 0.946 | 0.949 | 0.900 | 0.997 |
| RSAD2_IFIT1_HERC6_FAM20A_MMP8 | 0.850 | 0.840 | 0.861 | 0.879 | 0.809 | 0.949 | 0.925 | 0.842 | 1 |
| RSAD2_IFI27_OAS1_ISG15_HERC6 | 0.850 | 0.840 | 0.860 | 0.881 | 0.821 | 0.941 | 0.888 | 0.810 | 0.966 |
| RSAD2_SIGLEC1_ISG15_HERC6_MMP8 | 0.850 | 0.840 | 0.860 | 0.880 | 0.813 | 0.946 | 0.912 | 0.844 | 0.980 |
| IFIT1_IFI44L_OLAH_RETN_MMP8 | 0.850 | 0.840 | 0.861 | 0.879 | 0.804 | 0.954 | 0.962 | 0.922 | 1 |
| OAS1_IFIT1_ISG15_FAM20A_RETN | 0.850 | 0.840 | 0.861 | 0.881 | 0.807 | 0.955 | 0.941 | 0.866 | 1 |
| OAS1_IFI44L_SIGLEC1_RETN_MMP8 | 0.850 | 0.839 | 0.861 | 0.874 | 0.798 | 0.951 | 0.951 | 0.902 | 1 |
| RSAD2_IFI27_OAS1_IFIT1_MMP8 | 0.850 | 0.840 | 0.860 | 0.876 | 0.804 | 0.947 | 0.912 | 0.837 | 0.987 |
| OAS1_IFIT1_HERC6_SLC1A2_FAM20A | 0.850 | 0.840 | 0.860 | 0.882 | 0.816 | 0.948 | 0.929 | 0.845 | 1 |
| IFI27_OAS1_IFI44L_ISG15_HERC6 | 0.850 | 0.840 | 0.861 | 0.879 | 0.818 | 0.939 | 0.907 | 0.837 | 0.976 |
| IFIT1_IFI44L_ISG15_OLAH_RETN | 0.850 | 0.840 | 0.860 | 0.880 | 0.807 | 0.954 | 0.950 | 0.902 | 0.998 |
| RSAD2_OAS1_SIGLEC1_RETN_MMP8 | 0.850 | 0.839 | 0.861 | 0.876 | 0.800 | 0.952 | 0.949 | 0.900 | 0.998 |
| RSAD2_OAS1_HERC6_SLC1A2_RETN | 0.850 | 0.840 | 0.860 | 0.884 | 0.816 | 0.952 | 0.937 | 0.871 | 1 |

Performance obtained for the identification of the viral or bacterial nature of the infection on the basis of the six-gene combinations:

TABLE 10

| | AUC | | | AUC | CI 95% | | AUC | CI 95% | |
|---|---|---|---|---|---|---|---|---|---|
| Model | model | CI 95% | | Train | Train | | Test | Test | |
| IFI27 IFIT1 SIGLEC1 HERC6 OLAH FAM20A | 0.908 | 0.899 | 0.916 | 0.930 | 0.875 | 0.986 | 0.946 | 0.884 | 1 |
| IFI27 IFI44L SIGLEC1 IL1R2 FAM20A MMP8 | 0.907 | 0.899 | 0.914 | 0.931 | 0.880 | 0.983 | 0.952 | 0.885 | 1 |
| IFI27 IFIT1 SIGLEC1 IL1R2 FAM20A MMP8 | 0.906 | 0.898 | 0.914 | 0.932 | 0.880 | 0.984 | 0.957 | 0.894 | 1 |
| IFI27 OAS1 SIGLEC1 IL1R2 FAM20A MMP8 | 0.906 | 0.898 | 0.914 | 0.932 | 0.879 | 0.984 | 0.952 | 0.888 | 1 |
| RSAD2 IFI27 SIGLEC1 IL1R2 FAM20A MMP8 | 0.906 | 0.898 | 0.914 | 0.933 | 0.882 | 0.985 | 0.957 | 0.892 | 1 |
| IFI27 SIGLEC1 IL1R2 OLAH FAM20A MMP8 | 0.906 | 0.898 | 0.914 | 0.930 | 0.876 | 0.983 | 0.953 | 0.893 | 1 |
| IFI27 IFIT1 SIGLEC1 HERC6 IL1R2 FAM20A | 0.906 | 0.898 | 0.913 | 0.932 | 0.881 | 0.983 | 0.948 | 0.880 | 1 |
| IFI27 SIGLEC1 ISG15 IL1R2 FAM20A MMP8 | 0.905 | 0.897 | 0.913 | 0.932 | 0.880 | 0.984 | 0.953 | 0.885 | 1 |
| IFI27 IFIT1 SIGLEC1 OLAH FAM20A MMP8 | 0.905 | 0.896 | 0.914 | 0.927 | 0.869 | 0.985 | 0.957 | 0.902 | 1 |
| IFI27 OAS1 SIGLEC1 HERC6 OLAH FAM20A | 0.905 | 0.896 | 0.914 | 0.927 | 0.872 | 0.983 | 0.938 | 0.872 | 1 |
| IFI27 OAS1 SIGLEC1 HERC6 IL1R2 FAM20A | 0.905 | 0.896 | 0.913 | 0.930 | 0.878 | 0.983 | 0.948 | 0.881 | 1 |
| IFI27 SIGLEC1 HERC6 IL1R2 FAM20A MMP8 | 0.905 | 0.896 | 0.913 | 0.930 | 0.877 | 0.983 | 0.954 | 0.886 | 1 |
| RSAD2 IFI27 SIGLEC1 HERC6 IL1R2 FAM20A | 0.904 | 0.896 | 0.912 | 0.931 | 0.877 | 0.984 | 0.948 | 0.882 | 1 |
| IFI27 SIGLEC1 ISG15 OLAH FAM20A MMP8 | 0.904 | 0.895 | 0.913 | 0.924 | 0.864 | 0.984 | 0.950 | 0.891 | 1 |
| RSAD2 IFI27 HERC6 OLAH FAM20A MMP8 | 0.904 | 0.895 | 0.912 | 0.929 | 0.876 | 0.982 | 0.934 | 0.865 | 1 |
| IFI27 HERC6 IL1R2 OLAH FAM20A MMP8 | 0.904 | 0.895 | 0.912 | 0.932 | 0.880 | 0.983 | 0.935 | 0.863 | 1 |
| IFI27 IFI44L SIGLEC1 HERC6 IL1R2 FAM20A | 0.904 | 0.895 | 0.912 | 0.929 | 0.876 | 0.983 | 0.946 | 0.876 | 1 |
| RSAD2 IFI27 SIGLEC1 HERC6 OLAH FAM20A | 0.904 | 0.895 | 0.912 | 0.930 | 0.875 | 0.986 | 0.942 | 0.878 | 1 |
| IFI27 IFIT1 SIGLEC1 HERC6 FAM20A RETN | 0.903 | 0.895 | 0.912 | 0.928 | 0.873 | 0.983 | 0.940 | 0.867 | 1 |
| IFI27 IFIT1 SIGLEC1 IL1R2 OLAH FAM20A | 0.903 | 0.895 | 0.911 | 0.927 | 0.871 | 0.984 | 0.948 | 0.886 | 1 |
| IFI27 IFI44L IL1R2 OLAH FAM20A MMP8 | 0.903 | 0.895 | 0.912 | 0.929 | 0.874 | 0.983 | 0.932 | 0.859 | 1 |
| IFI27 SIGLEC1 IL1R2 FAM20A RETN MMP8 | 0.903 | 0.894 | 0.912 | 0.932 | 0.879 | 0.986 | 0.957 | 0.889 | 1 |
| IFI27 OAS1 HERC6 OLAH FAM20A MMP8 | 0.903 | 0.894 | 0.912 | 0.927 | 0.873 | 0.981 | 0.933 | 0.863 | 1 |
| IFI27 IFI44L SIGLEC1 OLAH FAM20A MMP8 | 0.903 | 0.894 | 0.912 | 0.924 | 0.864 | 0.984 | 0.955 | 0.898 | 1 |
| RSAD2 IFI27 SIGLEC1 OLAH FAM20A MMP8 | 0.903 | 0.894 | 0.912 | 0.927 | 0.869 | 0.985 | 0.951 | 0.895 | 1 |
| IFI27 IFIT1 SIGLEC1 ISG15 OLAH FAM20A | 0.903 | 0.894 | 0.911 | 0.926 | 0.868 | 0.985 | 0.941 | 0.879 | 1 |
| RSAD2 IFI27 HERC6 IL1R2 OLAH FAM20A | 0.903 | 0.895 | 0.911 | 0.929 | 0.877 | 0.982 | 0.929 | 0.857 | 1 |
| IFI27 IFIT1 IFI44L SIGLEC1 OLAH FAM20A | 0.903 | 0.894 | 0.911 | 0.925 | 0.866 | 0.984 | 0.952 | 0.897 | 1 |
| IFI27 ISG15 IL1R2 OLAH FAM20A MMP8 | 0.903 | 0.895 | 0.911 | 0.927 | 0.875 | 0.980 | 0.922 | 0.844 | 0.999 |
| IFI27 OAS1 HERC6 IL1R2 OLAH FAM20A | 0.903 | 0.894 | 0.911 | 0.930 | 0.878 | 0.981 | 0.927 | 0.852 | 1 |
| IFI27 ISG15 HERC6 OLAH FAM20A MMP8 | 0.902 | 0.893 | 0.911 | 0.925 | 0.868 | 0.982 | 0.937 | 0.872 | 1 |
| IFI27 IFI44L SIGLEC1 ISG15 OLAH FAM20A | 0.902 | 0.894 | 0.911 | 0.924 | 0.865 | 0.983 | 0.945 | 0.884 | 1 |
| IFI27 OAS1 SIGLEC1 OLAH FAM20A MMP8 | 0.902 | 0.893 | 0.911 | 0.923 | 0.862 | 0.984 | 0.950 | 0.893 | 1 |
| IFI27 OAS1 IFIT1 HERC6 OLAH FAM20A | 0.902 | 0.893 | 0.911 | 0.927 | 0.873 | 0.982 | 0.930 | 0.859 | 1 |
| RSAD2 IFI27 OAS1 HERC6 OLAH FAM20A | 0.902 | 0.893 | 0.911 | 0.927 | 0.873 | 0.981 | 0.930 | 0.859 | 1 |
| IFI27 ISG15 HERC6 IL1R2 OLAH FAM20A | 0.902 | 0.894 | 0.910 | 0.929 | 0.875 | 0.982 | 0.928 | 0.855 | 1 |
| IFI27 IFI44L SIGLEC1 ISG15 IL1R2 FAM20A | 0.902 | 0.894 | 0.910 | 0.928 | 0.874 | 0.982 | 0.947 | 0.880 | 1 |
| IFI27 IFI44L SIGLEC1 IL1R2 OLAH FAM20A | 0.902 | 0.894 | 0.910 | 0.926 | 0.870 | 0.982 | 0.951 | 0.889 | 1 |
| IFI27 IFIT1 IFI44L SIGLEC1 IL1R2 FAM20A | 0.902 | 0.894 | 0.910 | 0.927 | 0.872 | 0.982 | 0.949 | 0.886 | 1 |
| IFI27 IFIT1 HERC6 IL1R2 OLAH FAM20A | 0.902 | 0.894 | 0.910 | 0.929 | 0.877 | 0.982 | 0.928 | 0.853 | 1 |
| IFI27 OAS1 IFIT1 SIGLEC1 OLAH FAM20A | 0.902 | 0.893 | 0.910 | 0.923 | 0.863 | 0.983 | 0.950 | 0.891 | 1 |
| IFI27 OAS1 IFI44L HERC6 OLAH FAM20A | 0.902 | 0.893 | 0.911 | 0.926 | 0.870 | 0.982 | 0.933 | 0.862 | 1 |
| IFI27 IFIT1 IL1R2 OLAH FAM20A MMP8 | 0.902 | 0.893 | 0.910 | 0.929 | 0.876 | 0.982 | 0.935 | 0.866 | 1 |
| IFI27 IFI44L SIGLEC1 HERC6 OLAH FAM20A | 0.902 | 0.893 | 0.911 | 0.922 | 0.862 | 0.982 | 0.950 | 0.886 | 1 |
| IFI27 IFIT1 IFI44L HERC6 OLAH FAM20A | 0.902 | 0.893 | 0.911 | 0.925 | 0.869 | 0.982 | 0.933 | 0.863 | 1 |
| RSAD2 IFI27 IFI44L HERC6 OLAH FAM20A | 0.901 | 0.892 | 0.910 | 0.929 | 0.875 | 0.983 | 0.932 | 0.863 | 1.000 |
| IFI27 IFIT1 SIGLEC1 ISG15 IL1R2 FAM20A | 0.901 | 0.894 | 0.909 | 0.927 | 0.872 | 0.983 | 0.948 | 0.883 | 1 |
| IFI27 SIGLEC1 ISG15 IL1R2 OLAH FAM20A | 0.901 | 0.893 | 0.910 | 0.928 | 0.872 | 0.983 | 0.945 | 0.881 | 1 |
| IFI27 SIGLEC1 ISG15 HERC6 OLAH FAM20A | 0.901 | 0.892 | 0.911 | 0.923 | 0.863 | 0.983 | 0.945 | 0.882 | 1 |
| IFI27 IFIT1 HERC6 OLAH FAM20A MMP8 | 0.901 | 0.892 | 0.910 | 0.924 | 0.867 | 0.981 | 0.938 | 0.873 | 1 |
| IFI27 IFI44L ISG15 HERC6 OLAH FAM20A | 0.901 | 0.892 | 0.910 | 0.925 | 0.868 | 0.982 | 0.935 | 0.868 | 1 |
| IFI27 IFI44L HERC6 IL1R2 OLAH FAM20A | 0.901 | 0.893 | 0.910 | 0.928 | 0.874 | 0.982 | 0.935 | 0.863 | 1 |
| IFI27 OAS1 SIGLEC1 IL1R2 OLAH FAM20A | 0.901 | 0.893 | 0.910 | 0.924 | 0.866 | 0.982 | 0.948 | 0.886 | 1 |
| IFI27 OAS1 ISG15 HERC6 OLAH FAM20A | 0.901 | 0.892 | 0.910 | 0.929 | 0.874 | 0.983 | 0.927 | 0.854 | 1 |
| IFI27 OAS1 IFIT1 SIGLEC1 IL1R2 FAM20A | 0.901 | 0.893 | 0.909 | 0.927 | 0.871 | 0.983 | 0.950 | 0.889 | 1 |
| RSAD2 IFI27 IL1R2 OLAH FAM20A MMP8 | 0.901 | 0.893 | 0.909 | 0.929 | 0.876 | 0.982 | 0.930 | 0.857 | 1 |
| RSAD2 IFI27 IFIT1 HERC6 OLAH FAM20A | 0.901 | 0.892 | 0.910 | 0.927 | 0.872 | 0.981 | 0.932 | 0.862 | 1 |
| IFI27 OAS1 SIGLEC1 HERC6 FAM20A RETN | 0.901 | 0.892 | 0.910 | 0.928 | 0.873 | 0.983 | 0.938 | 0.864 | 1 |
| IFI27 OAS1 SIGLEC1 ISG15 OLAH FAM20A | 0.901 | 0.892 | 0.910 | 0.923 | 0.863 | 0.983 | 0.947 | 0.887 | 1 |
| IFI27 SIGLEC1 HERC6 IL1R2 OLAH FAM20A | 0.901 | 0.892 | 0.909 | 0.925 | 0.867 | 0.982 | 0.950 | 0.889 | 1 |
| IFI27 OAS1 IL1R2 OLAH FAM20A MMP8 | 0.901 | 0.892 | 0.909 | 0.929 | 0.876 | 0.982 | 0.932 | 0.859 | 1 |
| IFI27 IFIT1 ISG15 HERC6 OLAH FAM20A | 0.901 | 0.892 | 0.910 | 0.926 | 0.869 | 0.982 | 0.935 | 0.868 | 1 |
| IFI27 IFIT1 SIGLEC1 IL1R2 FAM20A RETN | 0.901 | 0.892 | 0.909 | 0.927 | 0.871 | 0.982 | 0.949 | 0.886 | 1 |
| IFI27 IFIT1 SIGLEC1 OLAH FAM20A RETN | 0.900 | 0.892 | 0.909 | 0.923 | 0.863 | 0.983 | 0.950 | 0.891 | 1 |
| RSAD2 IFI27 ISG15 HERC6 OLAH FAM20A | 0.900 | 0.892 | 0.909 | 0.927 | 0.873 | 0.982 | 0.929 | 0.859 | 1.000 |
| IFI27 SIGLEC1 ISG15 HERC6 IL1R2 FAM20A | 0.900 | 0.892 | 0.909 | 0.927 | 0.873 | 0.982 | 0.949 | 0.881 | 1 |
| IFI27 SIGLEC1 HERC6 OLAH FAM20A MMP8 | 0.900 | 0.891 | 0.910 | 0.921 | 0.859 | 0.984 | 0.953 | 0.895 | 1 |
| RSAD2 IFI27 IFIT1 SIGLEC1 OLAH FAM20A | 0.900 | 0.892 | 0.909 | 0.924 | 0.865 | 0.984 | 0.951 | 0.893 | 1 |
| IFI27 OAS1 HERC6 OLAH FAM20A RETN | 0.900 | 0.891 | 0.909 | 0.931 | 0.880 | 0.983 | 0.935 | 0.865 | 1 |
| RSAD2 IFI27 SIGLEC1 IL1R2 OLAH FAM20A | 0.900 | 0.892 | 0.909 | 0.927 | 0.869 | 0.984 | 0.948 | 0.885 | 1 |
| RSAD2 IFI27 OAS1 SIGLEC1 IL1R2 FAM20A | 0.900 | 0.892 | 0.908 | 0.927 | 0.871 | 0.984 | 0.951 | 0.888 | 1 |
| IFI27 OAS1 IFI44L SIGLEC1 IL1R2 FAM20A | 0.900 | 0.892 | 0.908 | 0.928 | 0.874 | 0.982 | 0.950 | 0.887 | 1 |
| IFI27 ISG15 HERC6 OLAH FAM20A RETN | 0.900 | 0.891 | 0.909 | 0.925 | 0.868 | 0.982 | 0.936 | 0.869 | 1 |
| IFI27 IFI44L ISG15 OLAH FAM20A MMP8 | 0.900 | 0.891 | 0.909 | 0.922 | 0.863 | 0.982 | 0.936 | 0.870 | 1 |
| IFI27 IFI44L SIGLEC1 IL1R2 FAM20A RETN | 0.900 | 0.891 | 0.908 | 0.927 | 0.872 | 0.982 | 0.948 | 0.881 | 1 |
| IFI27 OAS1 HERC6 IL1R2 FAM20A MMP8 | 0.900 | 0.891 | 0.908 | 0.930 | 0.880 | 0.979 | 0.924 | 0.837 | 1 |
| IFI27 IFI44L HERC6 OLAH FAM20A MMP8 | 0.900 | 0.890 | 0.909 | 0.924 | 0.866 | 0.981 | 0.940 | 0.876 | 1 |
| IFI27 OAS1 IFI44L SIGLEC1 OLAH FAM20A | 0.900 | 0.891 | 0.908 | 0.923 | 0.863 | 0.982 | 0.951 | 0.892 | 1 |
| IFI27 IFI44L HERC6 IL1R2 FAM20A MMP8 | 0.900 | 0.891 | 0.908 | 0.929 | 0.879 | 0.980 | 0.927 | 0.841 | 1 |
| RSAD2 IFI27 HERC6 IL1R2 FAM20A MMP8 | 0.900 | 0.891 | 0.908 | 0.929 | 0.879 | 0.979 | 0.926 | 0.841 | 1 |
| RSAD2 IFI27 HERC6 OLAH FAM20A RETN | 0.899 | 0.891 | 0.908 | 0.929 | 0.876 | 0.982 | 0.936 | 0.868 | 1 |
| IFI27 OAS1 SIGLEC1 ISG15 IL1R2 FAM20A | 0.899 | 0.891 | 0.908 | 0.927 | 0.872 | 0.983 | 0.949 | 0.882 | 1 |
| IFI27 IFIT1 HERC6 OLAH FAM20A RETN | 0.899 | 0.891 | 0.908 | 0.927 | 0.872 | 0.982 | 0.934 | 0.863 | 1 |
| RSAD2 IFI27 SIGLEC1 HERC6 FAM20A RETN | 0.899 | 0.890 | 0.908 | 0.929 | 0.876 | 0.983 | 0.946 | 0.876 | 1 |
| RSAD2 IFI27 SIGLEC1 ISG15 OLAH FAM20A | 0.899 | 0.890 | 0.908 | 0.924 | 0.865 | 0.983 | 0.945 | 0.883 | 1 |
| RSAD2 IFI27 OAS1 SIGLEC1 OLAH FAM20A | 0.899 | 0.890 | 0.908 | 0.924 | 0.864 | 0.984 | 0.951 | 0.893 | 1 |
| RSAD2 IFI27 IFIT1 SIGLEC1 IL1R2 FAM20A | 0.899 | 0.891 | 0.907 | 0.926 | 0.870 | 0.982 | 0.949 | 0.886 | 1 |
| RSAD2 IFI27 IFI44L SIGLEC1 IL1R2 FAM20A | 0.899 | 0.891 | 0.907 | 0.928 | 0.873 | 0.982 | 0.950 | 0.886 | 1 |
| IFI27 OAS1 SIGLEC1 IL1R2 FAM20A RETN | 0.899 | 0.890 | 0.908 | 0.928 | 0.872 | 0.984 | 0.950 | 0.886 | 1 |
| IFI27 SIGLEC1 ISG15 OLAH FAM20A RETN | 0.899 | 0.890 | 0.908 | 0.921 | 0.860 | 0.982 | 0.949 | 0.889 | 1 |
| IFI27 SIGLEC1 OLAH FAM20A RETN MMP8 | 0.899 | 0.889 | 0.908 | 0.924 | 0.865 | 0.984 | 0.957 | 0.899 | 1 |
| IFI27 HERC6 OLAH FAM20A RETN MMP8 | 0.899 | 0.889 | 0.908 | 0.927 | 0.869 | 0.984 | 0.943 | 0.878 | 1 |
| RSAD2 IFI27 SIGLEC1 IL1R2 FAM20A RETN | 0.899 | 0.890 | 0.907 | 0.929 | 0.874 | 0.984 | 0.948 | 0.881 | 1 |
| IFI27 OAS1 IFI44L HERC6 IL1R2 FAM20A | 0.899 | 0.891 | 0.907 | 0.926 | 0.875 | 0.978 | 0.926 | 0.842 | 1 |
| RSAD2 IFI27 SIGLEC1 ISG15 IL1R2 FAM20A | 0.899 | 0.890 | 0.907 | 0.928 | 0.873 | 0.983 | 0.946 | 0.878 | 1 |
| IFI27 IFI44L ISG15 HERC6 IL1R2 FAM20A | 0.899 | 0.890 | 0.907 | 0.927 | 0.876 | 0.978 | 0.930 | 0.849 | 1 |
| IFI27 IFI44L ISG15 IL1R2 OLAH FAM20A | 0.899 | 0.890 | 0.907 | 0.923 | 0.866 | 0.980 | 0.929 | 0.858 | 1 |
| IFI27 SIGLEC1 ISG15 IL1R2 FAM20A RETN | 0.898 | 0.890 | 0.907 | 0.928 | 0.873 | 0.983 | 0.950 | 0.883 | 1 |
| IFI27 IFIT1 HERC6 IL1R2 FAM20A MMP8 | 0.898 | 0.890 | 0.907 | 0.929 | 0.879 | 0.980 | 0.934 | 0.854 | 1 |
| IFI27 IFIT1 IFI44L HERC6 IL1R2 FAM20A | 0.898 | 0.890 | 0.907 | 0.927 | 0.876 | 0.978 | 0.932 | 0.851 | 1 |
| IFI27 IL1R2 OLAH FAM20A RETN MMP8 | 0.898 | 0.890 | 0.907 | 0.929 | 0.875 | 0.982 | 0.925 | 0.849 | 1 |
| RSAD2 IFI27 IFI44L SIGLEC1 OLAH FAM20A | 0.898 | 0.890 | 0.907 | 0.925 | 0.867 | 0.984 | 0.951 | 0.892 | 1 |
| IFI27 OAS1 SIGLEC1 OLAH FAM20A RETN | 0.898 | 0.889 | 0.907 | 0.924 | 0.865 | 0.983 | 0.948 | 1.886 | 1 |
| IFI27 IFI44L ISG15 IL1R2 FAM20A MMP8 | 0.898 | 0.890 | 0.906 | 0.927 | 0.874 | 0.979 | 0.926 | 0.841 | 1 |
| IFI27 OAS1 ISG15 HERC6 IL1R2 FAM20A | 0.898 | 0.890 | 0.906 | 0.927 | 0.877 | 0.978 | 0.923 | 0.837 | 1 |
| RSAD2 IFI27 IFI44L HERC6 IL1R2 FAM20A | 0.898 | 0.890 | 0.907 | 0.928 | 0.877 | 0.979 | 0.929 | 0.847 | 1 |
| IFI27 ISG15 HERC6 IL1R2 FAM20A MMP8 | 0.898 | 0.889 | 0.907 | 0.929 | 0.879 | 0.979 | 0.926 | 0.841 | 1 |
| IFI27 IFIT1 SIGLEC1 SLC1A2 OLAH FAM20A | 0.898 | 0.889 | 0.907 | 0.930 | 0.876 | 0.983 | 0.942 | 0.878 | 1 |
| IFI27 OAS1 HERC6 IL1R2 FAM20A RETN | 0.898 | 0.890 | 0.906 | 0.932 | 0.881 | 0.982 | 0.928 | 0.847 | 1 |
| RSAD2 IFI27 HERC6 IL1R2 FAM20A RETN | 0.898 | 0.889 | 0.906 | 0.931 | 0.880 | 0.982 | 0.930 | 0.850 | 1 |
| RSAD2 IFI27 OAS1 HERC6 IL1R2 FAM20A | 0.898 | 0.890 | 0.906 | 0.930 | 0.880 | 0.979 | 0.923 | 0.837 | 1 |
| IFI27 IFIT1 IFI44L OLAH FAM20A MMP8 | 0.898 | 0.889 | 0.907 | 0.921 | 0.861 | 0.981 | 0.938 | 0.873 | 1 |
| IFI27 OAS1 IFIT1 HERC6 IL1R2 FAM20A | 0.898 | 0.889 | 0.906 | 0.927 | 0.876 | 0.979 | 0.926 | 0.843 | 1 |
| IFI27 HERC6 IL1R2 OLAH FAM20A RETN | 0.898 | 0.889 | 0.906 | 0.926 | 0.871 | 0.982 | 0.939 | 0.870 | 1 |
| IFI27 SIGLEC1 ISG15 SLC1A2 OLAH FAM20A | 0.898 | 0.889 | 0.907 | 0.930 | 0.878 | 0.983 | 0.937 | 0.868 | 1 |
| IFI27 IFIT1 ISG15 OLAH FAM20A MMP8 | 0.898 | 0.889 | 0.907 | 0.921 | 0.861 | 0.981 | 0.936 | 0.871 | 1 |
| IFI27 IFI44L SIGLEC1 OLAH FAM20A RETN | 0.898 | 0.889 | 0.906 | 0.920 | 0.859 | 0.982 | 0.953 | 0.895 | 1 |
| IFI27 HERC6 IL1R2 FAM20A RETN MMP8 | 0.898 | 0.889 | 0.907 | 0.931 | 0.879 | 0.982 | 0.932 | 0.851 | 1 |
| RSAD2 IFI27 SIGLEC1 OLAH FAM20A RETN | 0.898 | 0.889 | 0.907 | 0.924 | 0.865 | 0.984 | 0.950 | 0.891 | 1 |
| IFI27 SIGLEC1 SLC1A2 IL1R2 FAM20A MMP8 | 0.898 | 0.889 | 0.906 | 0.936 | 0.891 | 0.981 | 0.954 | 0.885 | 1 |
| IFI27 IFIT1 SIGLEC1 ISG15 FAM20A RETN | 0.898 | 0.889 | 0.906 | 0.920 | 0.861 | 0.980 | 0.946 | 0.879 | 1 |
| IFI27 IFI44L HERC6 OLAH FAM20A RETN | 0.898 | 0.889 | 0.907 | 0.923 | 0.866 | 0.981 | 0.936 | 0.866 | 1 |
| IFI27 IFI44L HERC6 IL1R2 FAM20A RETN | 0.898 | 0.889 | 0.906 | 0.931 | 0.880 | 0.982 | 0.929 | 0.849 | 1 |
| IFI27 IFIT1 IFI44L IL1R2 FAM20A MMP8 | 0.898 | 0.889 | 0.906 | 0.926 | 0.873 | 0.978 | 0.936 | 0.858 | 1 |
| IFI27 ISG15 OLAH FAM20A RETN MMP8 | 0.898 | 0.888 | 0.907 | 0.924 | 0.868 | 0.981 | 0.930 | 0.858 | 1 |
| IFI27 SIGLEC1 IL1R2 OLAH FAM20A RETN | 0.897 | 0.889 | 0.906 | 0.924 | 0.866 | 0.982 | 0.950 | 0.890 | 1 |
| IFI27 ISG15 IL1R2 OLAH FAM20A RETN | 0.897 | 0.889 | 0.906 | 0.926 | 0.871 | 0.981 | 0.925 | 0.849 | 1 |
| RSAD2 IFI27 IFIT1 HERC6 IL1R2 FAM20A | 0.897 | 0.889 | 0.906 | 0.928 | 0.877 | 0.979 | 0.930 | 0.850 | 1 |
| IFI27 SIGLEC1 HERC6 IL1R2 FAM20A RETN | 0.897 | 0.888 | 0.906 | 0.927 | 0.872 | 0.982 | 0.950 | 0.883 | 1 |
| IFI27 OAS1 IFIT1 SIGLEC1 HERC6 RETN | 0.897 | 0.889 | 0.906 | 0.919 | 0.862 | 0.977 | 0.942 | 0.883 | 1 |
| IFI27 IFIT1 IFI44L SIGLEC1 FAM20A RETN | 0.897 | 0.888 | 0.906 | 0.920 | 0.860 | 0.979 | 0.947 | 0.880 | 1 |
| IFI27 ISG15 HERC6 IL1R2 FAM20A RETN | 0.897 | 0.889 | 0.906 | 0.931 | 0.880 | 0.982 | 0.929 | 0.849 | 1 |
| IFI27 IFIT1 IFI44L IL1R2 OLAH FAM20A | 0.897 | 0.889 | 0.906 | 0.923 | 0.866 | 0.981 | 0.932 | 0.860 | 1 |
| RSAD2 IFI27 OAS1 OLAH FAM20A MMP8 | 0.897 | 0.888 | 0.906 | 0.922 | 0.863 | 0.980 | 0.935 | 0.869 | 1 |
| IFI27 SLC1A2 IL1R2 OLAH FAM20A MMP8 | 0.897 | 0.888 | 0.906 | 0.930 | 0.881 | 0.979 | 0.920 | 0.839 | 1 |
| IFI27 IFIT1 SIGLEC1 FAM20A RETN MMP8 | 0.897 | 0.888 | 0.906 | 0.923 | 0.864 | 0.982 | 0.948 | 0.876 | 1 |
| IFI27 OAS1 ISG15 OLAH FAM20A MMP8 | 0.897 | 0.888 | 0.906 | 0.920 | 0.861 | 0.980 | 0.934 | 0.867 | 1 |
| IFI27 IFI44L IL1R2 FAM20A RETN MMP8 | 0.897 | 0.888 | 0.906 | 0.928 | 0.875 | 0.981 | 0.929 | 0.848 | 1 |
| IFI27 OAS1 IFIT1 IL1R2 FAM20A MMP8 | 0.897 | 0.889 | 0.905 | 0.925 | 0.873 | 0.978 | 0.933 | 0.854 | 1 |
| IFI27 SIGLEC1 SLC1A2 OLAH FAM20A MMP8 | 0.897 | 0.888 | 0.906 | 0.927 | 0.872 | 0.983 | 0.952 | 0.890 | 1 |
| IFIT1 SIGLEC1 HERC6 IL1R2 FAM20A MMP8 | 0.897 | 0.888 | 0.905 | 0.923 | 0.869 | 0.978 | 0.963 | 0.907 | 1 |
| IFI27 IFIT1 SIGLEC1 ISG15 HERC6 FAM20A | 0.897 | 0.888 | 0.905 | 0.921 | 0.861 | 0.981 | 0.942 | 0.867 | 1 |
| IFI27 OAS1 IFIT1 OLAH FAM20A MMP8 | 0.897 | 0.888 | 0.905 | 0.921 | 0.862 | 0.981 | 0.940 | 0.877 | 1 |
| IFI27 OAS1 SIGLEC1 SLC1A2 OLAH FAM20A | 0.897 | 0.888 | 0.906 | 0.928 | 0.872 | 0.984 | 0.941 | 0.878 | 1 |
| RSAD2 IFI27 ISG15 HERC6 IL1R2 FAM20A | 0.897 | 0.888 | 0.905 | 0.927 | 0.877 | 0.978 | 0.926 | 0.843 | 1 |
| IFI27 OAS1 IFIT1 IL1R2 OLAH FAM20A | 0.896 | 0.888 | 0.905 | 0.924 | 0.867 | 0.981 | 0.933 | 0.863 | 1 |
| IFI27 IFIT1 ISG15 IL1R2 OLAH FAM20A | 0.896 | 0.888 | 0.905 | 0.924 | 0.867 | 0.981 | 0.932 | 0.861 | 1 |
| IFI27 IFI44L SIGLEC1 HERC6 FAM20A RETN | 0.896 | 0.888 | 0.905 | 0.924 | 0.867 | 0.982 | 0.945 | 0.869 | 1 |
| IFI27 OAS1 HERC6 OLAH RETN MMP8 | 0.896 | 0.888 | 0.905 | 0.924 | 0.868 | 0.980 | 0.943 | 0.884 | 1 |
| IFI27 IFIT1 HERC6 IL1R2 FAM20A RETN | 0.896 | 0.888 | 0.905 | 0.931 | 0.880 | 0.982 | 0.929 | 0.849 | 1 |
| IFI27 IFI44L SIGLEC1 HERC6 IL1R2 OLAH | 0.896 | 0.888 | 0.904 | 0.927 | 0.879 | 0.976 | 0.942 | 0.890 | 0.994 |
| IFI27 SIGLEC1 HERC6 OLAH FAM20A RETN | 0.896 | 0.887 | 0.906 | 0.919 | 0.858 | 0.981 | 0.950 | 0.890 | 1 |
| IFI27 IFI44L OLAH FAM20A RETN MMP8 | 0.896 | 0.887 | 0.906 | 0.924 | 0.864 | 0.983 | 0.941 | 0.875 | 1 |
| IFI27 OAS1 HERC6 SLC1A2 OLAH FAM20A | 0.896 | 0.887 | 0.905 | 0.929 | 0.878 | 0.980 | 0.918 | 0.835 | 1 |
| IFI27 IFI44L SLC1A2 OLAH FAM20A MMP8 | 0.896 | 0.887 | 0.905 | 0.926 | 0.870 | 0.981 | 0.928 | 0.852 | 1 |
| RSAD2 IFI27 IFIT1 IL1R2 FAM20A MMP8 | 0.896 | 0.888 | 0.904 | 0.926 | 0.874 | 0.978 | 0.933 | 0.854 | 1 |
| IFI27 OAS1 SIGLEC1 ISG15 HERC6 RETN | 0.896 | 0.887 | 0.905 | 0.921 | 0.863 | 0.979 | 0.945 | 0.882 | 1 |
| IFI27 IFIT1 SIGLEC1 HERC6 FAM20A MMP8 | 0.896 | 0.888 | 0.905 | 0.921 | 0.861 | 0.980 | 0.939 | 0.864 | 1 |
| RSAD2 IFI27 OAS1 IL1R2 OLAH FAM20A | 0.896 | 0.888 | 0.905 | 0.923 | 0.866 | 0.980 | 0.930 | 0.858 | 1 |
| IFI27 IFIT1 ISG15 HERC6 IL1R2 FAM20A | 0.896 | 0.888 | 0.905 | 0.927 | 0.876 | 0.978 | 0.933 | 0.853 | 1 |
| IFI27 OAS1 SIGLEC1 HERC6 OLAH RETN | 0.896 | 0.888 | 0.905 | 0.923 | 0.867 | 0.978 | 0.946 | 0.890 | 1 |
| RSAD2 IFI27 IFI44L OLAH FAM20A MMP8 | 0.896 | 0.886 | 0.906 | 0.922 | 0.863 | 0.982 | 0.935 | 0.868 | 1 |
| RSAD2 IFI27 ISG15 OLAH FAM20A MMP8 | 0.896 | 0.887 | 0.905 | 0.921 | 0.861 | 0.981 | 0.933 | 0.865 | 1 |
| IFI27 OAS1 ISG15 IL1R2 OLAH FAM20A | 0.896 | 0.887 | 0.905 | 0.925 | 0.869 | 0.981 | 0.927 | 0.855 | 0.999 |
| IFI27 IFI44L SIGLEC1 HERC6 OLAH RETN | 0.896 | 0.887 | 0.905 | 0.922 | 0.866 | 0.978 | 0.948 | 0.896 | 1.000 |
| IFI27 IFIT1 IFI44L ISG15 OLAH FAM20A | 0.896 | 0.887 | 0.905 | 0.920 | 0.859 | 0.981 | 0.935 | 0.867 | 1 |
| IFI27 OAS1 ISG15 HERC6 FAM20A RETN | 0.896 | 0.887 | 0.905 | 0.924 | 0.867 | 0.980 | 0.935 | 0.857 | 1 |
| IFI27 OAS1 IFI44L OLAH FAM20A MMP8 | 0.896 | 0.886 | 0.905 | 0.922 | 0.862 | 0.982 | 0.935 | 0.867 | 1 |
| IFI27 IFIT1 IFI44L SIGLEC1 HERC6 FAM20A | 0.896 | 0.888 | 0.904 | 0.920 | 0.861 | 0.980 | 0.941 | 0.866 | 1 |
| RSAD2 IFI27 IFIT1 OLAH FAM20A MMP8 | 0.896 | 0.887 | 0.905 | 0.922 | 0.864 | 0.981 | 0.940 | 0.876 | 1 |
| RSAD2 IFI27 ISG15 IL1R2 OLAH FAM20A | 0.896 | 0.887 | 0.904 | 0.924 | 0.867 | 0.980 | 0.927 | 0.853 | 1 |
| IFI27 IFI44L ISG15 SLC1A2 OLAH FAM20A | 0.896 | 0.887 | 0.905 | 0.929 | 0.875 | 0.983 | 0.917 | 0.836 | 0.999 |
| RSAD2 IFI27 HERC6 SLC1A2 OLAH FAM20A | 0.896 | 0.887 | 0.905 | 0.928 | 0.875 | 0.980 | 0.922 | 0.841 | 1 |
| IFI27 IFI44L IL1R2 OLAH FAM20A RETN | 0.896 | 0.887 | 0.904 | 0.924 | 0.866 | 0.981 | 0.930 | 0.858 | 1 |
| IFI27 ISG15 SLC1A2 OLAH FAM20A MMP8 | 0.896 | 0.887 | 0.905 | 0.927 | 0.873 | 0.980 | 0.922 | 0.843 | 1 |
| IFI27 OAS1 IFIT1 HERC6 OLAH RETN | 0.896 | 0.887 | 0.904 | 0.922 | 0.867 | 0.978 | 0.939 | 0.878 | 1.000 |
| IFI27 OAS1 IFIT1 SIGLEC1 HERC6 FAM20A | 0.896 | 0.887 | 0.904 | 0.923 | 0.866 | 0.980 | 0.938 | 0.865 | 1 |
| RSAD2 IFI27 OAS1 SIGLEC1 HERC6 RETN | 0.896 | 0.887 | 0.904 | 0.918 | 0.859 | 0.978 | 0.949 | 0.888 | 1 |
| IFI27 OAS1 SIGLEC1 HERC6 IL1R2 RETN | 0.896 | 0.888 | 0.904 | 0.924 | 0.872 | 0.977 | 0.949 | 0.894 | 1 |
| IFI27 IFIT1 SIGLEC1 HERC6 OLAH RETN | 0.896 | 0.887 | 0.904 | 0.923 | 0.869 | 0.978 | 0.947 | 0.894 | 0.999 |
| IFI27 ISG15 HERC6 SLC1A2 OLAH FAM20A | 0.896 | 0.886 | 0.905 | 0.931 | 0.880 | 0.982 | 0.915 | 0.832 | 0.998 |
| IFI27 IFI44L SIGLEC1 ISG15 HERC6 OLAH | 0.895 | 0.887 | 0.904 | 0.925 | 0.873 | 0.977 | 0.945 | 0.892 | 0.997 |
| RSAD2 IFI27 HERC6 OLAH RETN MMP8 | 0.895 | 0.887 | 0.904 | 0.924 | 0.867 | 0.980 | 0.945 | 0.889 | 1 |
| IFI27 OAS1 IFI44L IL1R2 FAM20A MMP8 | 0.895 | 0.887 | 0.904 | 0.926 | 0.873 | 0.979 | 0.930 | 0.847 | 1 |
| IFI27 OAS1 IFI44L SIGLEC1 HERC6 RETN | 0.895 | 0.887 | 0.903 | 0.919 | 0.861 | 0.977 | 0.943 | 0.883 | 1 |
| IFI27 IFI44L ISG15 OLAH FAM20A RETN | 0.895 | 0.886 | 0.904 | 0.921 | 0.861 | 0.981 | 0.935 | 0.865 | 1 |
| RSAD2 IFI27 IFI44L IL1R2 FAM20A MMP8 | 0.895 | 0.887 | 0.904 | 0.926 | 0.874 | 0.979 | 0.928 | 0.845 | 1 |
| RSAD2 IFI27 SIGLEC1 SLC1A2 OLAH FAM20A | 0.895 | 0.886 | 0.904 | 0.927 | 0.871 | 0.984 | 0.943 | 0.879 | 1 |
| IFI27 IFI44L SIGLEC1 SLC1A2 OLAH FAM20A | 0.895 | 0.886 | 0.904 | 0.925 | 0.867 | 0.983 | 0.946 | 0.879 | 1 |
| IFI27 IFIT1 ISG15 IL1R2 FAM20A MMP8 | 0.895 | 0.887 | 0.904 | 0.927 | 0.875 | 0.979 | 0.933 | 0.854 | 1 |
| IFI27 IFIT1 OLAH FAM20A RETN MMP8 | 0.895 | 0.886 | 0.905 | 0.923 | 0.864 | 0.982 | 0.943 | 0.879 | 1 |
| IFI27 IFIT1 IL1R2 FAM20A RETN MMP8 | 0.895 | 0.886 | 0.904 | 0.929 | 0.875 | 0.982 | 0.937 | 0.860 | 1 |
| IFI27 OAS1 IFIT1 IFI44L OLAH FAM20A | 0.895 | 0.886 | 0.904 | 0.920 | 0.859 | 0.981 | 0.937 | 0.869 | 1 |
| IFI27 OAS1 SIGLEC1 FAM20A RETN MMP8 | 0.895 | 0.885 | 0.905 | 0.922 | 0.861 | 0.982 | 0.947 | 0.875 | 1 |
| RSAD2 IFI27 IFIT1 IL1R2 OLAH FAM20A | 0.895 | 0.887 | 0.904 | 0.924 | 0.867 | 0.981 | 0.933 | 0.863 | 1 |
| RSAD2 IFI27 SIGLEC1 HERC6 IL1R2 OLAH | 0.895 | 0.887 | 0.903 | 0.926 | 0.877 | 0.975 | 0.941 | 0.888 | 0.995 |
| IFI27 OAS1 IFI44L IL1R2 OLAH FAM20A | 0.895 | 0.886 | 0.904 | 0.923 | 0.866 | 0.981 | 0.929 | 0.856 | 1 |
| IFI27 IFIT1 HERC6 OLAH RETN MMP8 | 0.895 | 0.886 | 0.904 | 0.922 | 0.864 | 0.980 | 0.949 | 0.896 | 1 |
| IFI27 OAS1 IFIT1 SIGLEC1 FAM20A RETN | 0.895 | 0.886 | 0.904 | 0.919 | 0.860 | 0.979 | 0.946 | 0.878 | 1 |
| IFI27 IFI44L SIGLEC1 HERC6 OLAH MMP8 | 0.895 | 0.887 | 0.903 | 0.924 | 0.871 | 0.978 | 0.946 | 0.895 | 0.997 |
| IFI27 OAS1 SIGLEC1 HERC6 IL1R2 OLAH | 0.895 | 0.887 | 0.903 | 0.924 | 0.875 | 0.974 | 0.943 | 0.891 | 0.996 |
| IFI27 IFIT1 SIGLEC1 SLC1A2 IL1R2 FAM20A | 0.895 | 0.886 | 0.904 | 0.934 | 0.887 | 0.981 | 0.943 | 0.874 | 1 |
| RSAD2 IFI27 SLC1A2 OLAH FAM20A MMP8 | 0.895 | 0.886 | 0.904 | 0.924 | 0.869 | 0.980 | 0.925 | 0.848 | 1 |
| RSAD2 IFI27 IFIT1 SIGLEC1 HERC6 FAM20A | 0.895 | 0.886 | 0.904 | 0.922 | 0.865 | 0.979 | 0.941 | 0.871 | 1 |
| RSAD2 IFI27 SIGLEC1 HERC6 OLAH MMP8 | 0.895 | 0.887 | 0.903 | 0.922 | 0.869 | 0.974 | 0.937 | 0.881 | 0.993 |
| IFI27 OAS1 IFI44L ISG15 OLAH FAM20A | 0.895 | 0.886 | 0.904 | 0.920 | 0.860 | 0.981 | 0.935 | 0.868 | 1 |
| IFI27 IFI44L SIGLEC1 SLC1A2 IL1R2 FAM20A | 0.895 | 0.886 | 0.904 | 0.928 | 0.878 | 0.979 | 0.948 | 0.879 | 1 |
| IFI27 OAS1 OLAH FAM20A RETN MMP8 | 0.895 | 0.886 | 0.904 | 0.924 | 0.866 | 0.982 | 0.939 | 0.872 | 1 |
| IFI27 OAS1 SIGLEC1 SLC1A2 IL1R2 FAM20A | 0.895 | 0.886 | 0.904 | 0.932 | 0.884 | 0.980 | 0.943 | 0.873 | 1 |
| IFI27 OAS1 HERC6 IL1R2 OLAH MMP8 | 0.895 | 0.887 | 0.903 | 0.927 | 0.879 | 0.976 | 0.938 | 0.880 | 0.996 |
| IFI27 IFI44L SIGLEC1 IL1R2 OLAH MMP8 | 0.895 | 0.887 | 0.903 | 0.924 | 0.873 | 0.974 | 0.947 | 0.896 | 0.998 |
| IFI27 IFIT1 IFI44L HERC6 FAM20A RETN | 0.895 | 0.886 | 0.904 | 0.921 | 0.862 | 0.979 | 0.928 | 0.845 | 1 |
| IFI27 OAS1 IFIT1 SIGLEC1 HERC6 OLAH | 0.895 | 0.887 | 0.903 | 0.924 | 0.872 | 0.975 | 0.940 | 0.885 | 0.995 |
| IFI27 OAS1 IFI44L HERC6 FAM20A RETN | 0.895 | 0.886 | 0.904 | 0.923 | 0.864 | 0.981 | 0.935 | 0.856 | 1 |
| IFI27 OAS1 IFIT1 ISG15 OLAH FAM20A | 0.895 | 0.886 | 0.904 | 0.921 | 0.861 | 0.981 | 0.934 | 0.866 | 1 |
| IFI27 IFI44L HERC6 OLAH RETN MMP8 | 0.895 | 0.886 | 0.904 | 0.922 | 0.864 | 0.980 | 0.948 | 0.895 | 1 |
| IFI27 ISG15 SLC1A2 IL1R2 OLAH FAM20A | 0.895 | 0.886 | 0.903 | 0.929 | 0.877 | 0.980 | 0.916 | 0.833 | 0.999 |
| IFI27 IFIT1 IL1R2 OLAH FAM20A RETN | 0.895 | 0.886 | 0.903 | 0.924 | 0.868 | 0.981 | 0.932 | 0.860 | 1 |
| RSAD2 IFI27 IFI44L IL1R2 OLAH FAM20A | 0.895 | 0.886 | 0.904 | 0.924 | 0.866 | 0.981 | 0.929 | 0.856 | 1 |
| IFI27 IFIT1 SLC1A2 OLAH FAM20A MMP8 | 0.895 | 0.885 | 0.904 | 0.924 | 0.868 | 0.980 | 0.928 | 0.854 | 1 |
| RSAD2 IFI27 IL1R2 OLAH FAM20A RETN | 0.895 | 0.886 | 0.903 | 0.924 | 0.867 | 0.980 | 0.930 | 0.858 | 1 |
| IFI27 OAS1 SIGLEC1 HERC6 OLAH MMP8 | 0.895 | 0.886 | 0.903 | 0.923 | 0.871 | 0.976 | 0.939 | 0.883 | 0.996 |
| IFI27 IFI44L SIGLEC1 ISG15 IL1R2 OLAH | 0.894 | 0.887 | 0.902 | 0.923 | 0.873 | 0.974 | 0.943 | 0.891 | 0.996 |
| RSAD2 IFI27 OAS1 HERC6 OLAH RETN | 0.894 | 0.886 | 0.903 | 0.922 | 0.866 | 0.979 | 0.942 | 0.883 | 1 |
| IFI27 OAS1 IFIT1 HERC6 OLAH MMP8 | 0.894 | 0.886 | 0.903 | 0.923 | 0.871 | 0.975 | 0.935 | 0.876 | 0.994 |
| IFI27 IFIT1 IFI44L OLAH FAM20A RETN | 0.894 | 0.886 | 0.903 | 0.919 | 0.858 | 0.981 | 0.938 | 0.870 | 1 |
| RSAD2 IFI27 OAS1 IFI44L OLAH FAM20A | 0.894 | 0.885 | 0.903 | 0.921 | 0.861 | 0.981 | 0.933 | 0.863 | 1 |
| RSAD2 IFI27 HERC6 IL1R2 OLAH MMP8 | 0.894 | 0.886 | 0.902 | 0.927 | 0.878 | 0.975 | 0.938 | 0.882 | 0.994 |
| RSAD2 IFI27 IFIT1 IFI44L OLAH FAM20A | 0.894 | 0.885 | 0.903 | 0.923 | 0.863 | 0.983 | 0.933 | 0.862 | 1 |
| RSAD2 IFI27 OAS1 IL1R2 FAM20A MMP8 | 0.894 | 0.886 | 0.903 | 0.926 | 0.875 | 0.978 | 0.928 | 0.843 | 1 |
| RSAD2 IFI27 OAS1 ISG15 OLAH FAM20A | 0.894 | 0.885 | 0.903 | 0.920 | 0.860 | 0.980 | 0.934 | 0.866 | 1 |
| IFI27 OAS1 IFI44L SIGLEC1 HERC6 OLAH | 0.894 | 0.886 | 0.902 | 0.926 | 0.875 | 0.977 | 0.945 | 0.892 | 0.997 |
| RSAD2 IFI27 SIGLEC1 SLC1A2 IL1R2 FAM20A | 0.894 | 0.885 | 0.903 | 0.931 | 0.881 | 0.982 | 0.948 | 0.881 | 1 |
| IFI27 IFIT1 SIGLEC1 HERC6 OLAH MMP8 | 0.894 | 0.886 | 0.903 | 0.921 | 0.867 | 0.975 | 0.940 | 0.886 | 0.994 |
| IFI27 OAS1 IL1R2 OLAH FAM20A RETN | 0.894 | 0.886 | 0.903 | 0.924 | 0.868 | 0.981 | 0.930 | 0.858 | 1 |
| IFI27 IFI44L SLC1A2 IL1R2 OLAH FAM20A | 0.894 | 0.886 | 0.903 | 0.928 | 0.875 | 0.982 | 0.923 | 0.842 | 1 |
| RSAD2 IFI27 SIGLEC1 FAM20A RETN MMP8 | 0.894 | 0.884 | 0.904 | 0.926 | 0.868 | 0.984 | 0.947 | 0.875 | 1 |
| RSAD2 IFI27 IFI44L SIGLEC1 HERC6 OLAH | 0.894 | 0.886 | 0.902 | 0.924 | 0.872 | 0.977 | 0.947 | 0.897 | 0.997 |
| RSAD2 IFI27 OLAH FAM20A RETN MMP8 | 0.894 | 0.885 | 0.904 | 0.923 | 0.864 | 0.982 | 0.940 | 0.874 | 1 |
| IFI27 IFIT1 ISG15 OLAH FAM20A RETN | 0.894 | 0.885 | 0.903 | 0.920 | 0.861 | 0.980 | 0.933 | 0.862 | 1 |
| IFI27 IFI44L SIGLEC1 ISG15 FAM20A RETN | 0.894 | 0.885 | 0.903 | 0.920 | 0.860 | 0.979 | 0.946 | 0.874 | 1 |
| IFI27 HERC6 SLC1A2 OLAH FAM20A MMP8 | 0.894 | 0.885 | 0.903 | 0.927 | 0.873 | 0.981 | 0.924 | 0.844 | 1 |
| IFI27 SIGLEC1 SLC1A2 IL1R2 OLAH FAM20A | 0.894 | 0.885 | 0.903 | 0.926 | 0.871 | 0.981 | 0.946 | 0.880 | 1 |
| IFI27 OAS1 SIGLEC1 HERC6 RETN MMP8 | 0.894 | 0.885 | 0.903 | 0.919 | 0.859 | 0.979 | 0.950 | 0.890 | 1 |
| RSAD2 IFI27 IFI44L ISG15 OLAH FAM20A | 0.894 | 0.885 | 0.903 | 0.920 | 0.859 | 0.981 | 0.933 | 0.863 | 1 |
| IFI27 OAS1 IL1R2 FAM20A RETN MMP8 | 0.894 | 0.885 | 0.902 | 0.929 | 0.877 | 0.981 | 0.934 | 0.855 | 1 |
| IFI27 SIGLEC1 HERC6 SLC1A2 OLAH FAM20A | 0.894 | 0.884 | 0.903 | 0.926 | 0.869 | 0.983 | 0.945 | 0.877 | 1 |
| RSAD2 IFI27 SIGLEC1 HERC6 OLAH RETN | 0.894 | 0.885 | 0.902 | 0.922 | 0.868 | 0.977 | 0.942 | 0.886 | 0.999 |
| RSAD2 IFI27 OAS1 SIGLEC1 HERC6 OLAH | 0.894 | 0.885 | 0.902 | 0.923 | 0.871 | 0.975 | 0.938 | 0.881 | 0.995 |
| RSAD2 IFI27 OAS1 IFIT1 OLAH FAM20A | 0.894 | 0.885 | 0.903 | 0.920 | 0.860 | 0.981 | 0.937 | 0.869 | 1 |
| IFI27 SIGLEC1 ISG15 SLC1A2 IL1R2 FAM20A | 0.894 | 0.885 | 0.903 | 0.932 | 0.885 | 0.980 | 0.947 | 0.877 | 1 |
| IFI27 IFI44L ISG15 IL1R2 FAM20A RETN | 0.894 | 0.885 | 0.902 | 0.924 | 0.871 | 0.978 | 0.927 | 0.846 | 1 |
| IFI27 IFIT1 IFI44L SIGLEC1 HERC6 OLAH | 0.894 | 0.886 | 0.902 | 0.925 | 0.872 | 0.978 | 0.947 | 0.897 | 0.997 |
| IFI27 IFI44L HERC6 IL1R2 OLAH MMP8 | 0.894 | 0.886 | 0.902 | 0.927 | 0.878 | 0.976 | 0.937 | 0.880 | 0.993 |
| IFI27 IFIT1 SIGLEC1 HERC6 IL1R2 OLAH | 0.894 | 0.886 | 0.902 | 0.926 | 0.876 | 0.975 | 0.941 | 0.888 | 0.995 |
| RSAD2 IFI27 OAS1 HERC6 OLAH MMP8 | 0.894 | 0.885 | 0.902 | 0.923 | 0.871 | 0.975 | 0.934 | 0.874 | 0.993 |
| IFI27 OAS1 IFIT1 HERC6 FAM20A RETN | 0.894 | 0.884 | 0.903 | 0.923 | 0.865 | 0.981 | 0.934 | 0.853 | 1 |
| RSAD2 IFI27 OAS1 HERC6 IL1R2 OLAH | 0.894 | 0.885 | 0.902 | 0.928 | 0.880 | 0.976 | 0.938 | 0.880 | 0.996 |
| IFI27 IFI44L SIGLEC1 HERC6 IL1R2 RETN | 0.894 | 0.886 | 0.902 | 0.924 | 0.871 | 0.977 | 0.945 | 0.893 | 0.996 |
| IFI27 IFIT1 HERC6 SLC1A2 OLAH FAM20A | 0.894 | 0.884 | 0.903 | 0.928 | 0.875 | 0.980 | 0.924 | 0.844 | 1 |
| OAS1 IFIT1 SIGLEC1 IL1R2 FAM20A MMP8 | 0.894 | 0.885 | 0.902 | 0.920 | 0.863 | 0.977 | 0.963 | 0.909 | 1 |
| IFIT1 SIGLEC1 IL1R2 OLAH FAM20A MMP8 | 0.894 | 0.885 | 0.902 | 0.922 | 0.866 | 0.978 | 0.966 | 0.916 | 1 |
| IFI27 ISG15 HERC6 OLAH RETN MMP8 | 0.894 | 0.884 | 0.903 | 0.920 | 0.861 | 0.979 | 0.951 | 0.899 | 1 |
| IFI27 IFIT1 IFI44L SLC1A2 OLAH FAM20A | 0.894 | 0.885 | 0.902 | 0.926 | 0.871 | 0.982 | 0.921 | 0.839 | 1 |
| RSAD2 IFI27 IFIT1 ISG15 OLAH FAM20A | 0.894 | 0.885 | 0.903 | 0.920 | 0.860 | 0.980 | 0.935 | 0.867 | 1 |
| IFI27 OAS1 IFI44L HERC6 OLAH MMP8 | 0.894 | 0.885 | 0.902 | 0.925 | 0.874 | 0.976 | 0.934 | 0.874 | 0.994 |
| RSAD2 IFI27 IFIT1 HERC6 OLAH RETN | 0.894 | 0.885 | 0.902 | 0.923 | 0.868 | 0.978 | 0.937 | 0.876 | 0.998 |
| IFI27 IFI44L ISG15 HERC6 OLAH MMP8 | 0.894 | 0.885 | 0.902 | 0.923 | 0.871 | 0.976 | 0.932 | 0.871 | 0.992 |
| IFI27 IFIT1 IFI44L SIGLEC1 ISG15 FAM20A | 0.894 | 0.885 | 0.902 | 0.915 | 0.851 | 0.980 | 0.942 | 0.870 | 1 |
| RSAD2 IFI27 ISG15 SLC1A2 OLAH FAM20A | 0.894 | 0.885 | 0.903 | 0.927 | 0.872 | 0.981 | 0.916 | 0.834 | 0.999 |
| IFI27 OAS1 HERC6 IL1R2 OLAH RETN | 0.894 | 0.885 | 0.902 | 0.922 | 0.867 | 0.978 | 0.939 | 0.878 | 1.000 |
| RSAD2 IFI27 ISG15 HERC6 FAM20A RETN | 0.894 | 0.885 | 0.903 | 0.924 | 0.869 | 0.980 | 0.933 | 0.852 | 1 |
| IFI27 OAS1 SLC1A2 OLAH FAM20A MMP8 | 0.894 | 0.885 | 0.903 | 0.925 | 0.870 | 0.980 | 0.926 | 0.850 | 1 |
| IFI27 HERC6 SLC1A2 IL1R2 OLAH FAM20A | 0.894 | 0.885 | 0.902 | 0.930 | 0.879 | 0.980 | 0.928 | 0.849 | 1 |
| IFI27 OAS1 ISG15 HERC6 OLAH RETN | 0.894 | 0.885 | 0.902 | 0.922 | 0.867 | 0.978 | 0.940 | 0.880 | 1 |
| IFI27 IFIT1 ISG15 HERC6 FAM20A RETN | 0.894 | 0.884 | 0.903 | 0.923 | 0.866 | 0.980 | 0.934 | 0.854 | 1 |
| IFI27 IFIT1 IFI44L IL1R2 FAM20A RETN | 0.893 | 0.885 | 0.902 | 0.923 | 0.869 | 0.977 | 0.930 | 0.852 | 1 |
| IFI27 OAS1 ISG15 OLAH FAM20A RETN | 0.893 | 0.885 | 0.902 | 0.920 | 0.861 | 0.980 | 0.934 | 0.866 | 1 |
| IFI27 OAS1 IFIT1 IFI44L IL1R2 FAM20A | 0.893 | 0.885 | 0.902 | 0.922 | 0.868 | 0.976 | 0.928 | 0.848 | 1 |
| RSAD2 IFI27 IL1R2 FAM20A RETN MMP8 | 0.893 | 0.885 | 0.902 | 0.927 | 0.874 | 0.980 | 0.932 | 0.851 | 1 |
| IFI27 OAS1 IFIT1 HERC6 IL1R2 OLAH | 0.893 | 0.885 | 0.902 | 0.925 | 0.875 | 0.974 | 0.939 | 0.882 | 0.996 |
| IFI27 OAS1 IFIT1 OLAH FAM20A RETN | 0.893 | 0.884 | 0.902 | 0.918 | 0.857 | 0.979 | 0.935 | 0.864 | 1 |
| RSAD2 IFI27 OAS1 HERC6 FAM20A RETN | 0.893 | 0.884 | 0.903 | 0.923 | 0.865 | 0.981 | 0.935 | 0.855 | 1 |
| IFI27 ISG15 IL1R2 FAM20A RETN MMP8 | 0.893 | 0.885 | 0.902 | 0.926 | 0.875 | 0.976 | 0.920 | 0.833 | 1 |
| IFI27 OAS1 SIGLEC1 ISG15 FAM20A RETN | 0.893 | 0.884 | 0.903 | 0.917 | 0.855 | 0.979 | 0.946 | 0.876 | 1 |
| IFI27 OAS1 IFI44L HERC6 OLAH RETN | 0.893 | 0.885 | 0.902 | 0.923 | 0.868 | 0.977 | 0.935 | 0.872 | 0.998 |
| IFI27 SIGLEC1 HERC6 OLAH RETN MMP8 | 0.893 | 0.884 | 0.903 | 0.918 | 0.858 | 0.979 | 0.961 | 0.915 | 1 |
| IFI27 IFI44L ISG15 HERC6 IL1R2 OLAH | 0.893 | 0.885 | 0.901 | 0.927 | 0.877 | 0.976 | 0.934 | 0.874 | 0.993 |
| IFI27 IFI44L ISG15 HERC6 OLAH RETN | 0.893 | 0.884 | 0.902 | 0.921 | 0.864 | 0.977 | 0.941 | 0.885 | 0.997 |
| RSAD2 SIGLEC1 HERC6 IL1R2 FAM20A MMP8 | 0.893 | 0.885 | 0.902 | 0.920 | 0.864 | 0.976 | 0.961 | 0.902 | 1 |
| IFI27 SIGLEC1 ISG15 SLC1A2 OLAH MMP8 | 0.893 | 0.885 | 0.902 | 0.930 | 0.879 | 0.980 | 0.939 | 0.879 | 0.999 |
| IFI27 IFIT1 SIGLEC1 ISG15 HERC6 OLAH | 0.893 | 0.885 | 0.901 | 0.923 | 0.870 | 0.976 | 0.942 | 0.889 | 0.996 |
| IFI27 IFIT1 IFI44L HERC6 OLAH RETN | 0.893 | 0.884 | 0.902 | 0.920 | 0.864 | 0.977 | 0.943 | 0.888 | 0.999 |
| IFI27 IFIT1 ISG15 SLC1A2 OLAH FAM20A | 0.893 | 0.884 | 0.902 | 0.924 | 0.868 | 0.980 | 0.923 | 0.846 | 1.000 |
| IFI44L SIGLEC1 HERC6 IL1R2 FAM20A MMP8 | 0.893 | 0.884 | 0.902 | 0.919 | 0.862 | 0.976 | 0.959 | 0.897 | 1 |
| IFI27 OAS1 SIGLEC1 HERC6 IL1R2 MMP8 | 0.893 | 0.885 | 0.901 | 0.922 | 0.875 | 0.968 | 0.948 | 0.900 | 0.996 |
| RSAD2 IFI27 IFIT1 IFI44L IL1R2 FAM20A | 0.893 | 0.885 | 0.901 | 0.922 | 0.867 | 0.976 | 0.928 | 0.848 | 1 |
| IFI27 OAS1 ISG15 IL1R2 FAM20A MMP8 | 0.893 | 0.885 | 0.901 | 0.927 | 0.875 | 0.978 | 0.930 | 0.847 | 1 |
| IFI27 SIGLEC1 ISG15 IL1R2 OLAH MMP8 | 0.893 | 0.885 | 0.901 | 0.923 | 0.872 | 0.974 | 0.943 | 0.892 | 0.995 |
| IFI27 IFIT1 IFI44L ISG15 IL1R2 FAM20A | 0.893 | 0.884 | 0.901 | 0.923 | 0.869 | 0.977 | 0.928 | 0.848 | 1 |
| IFI27 OAS1 IFIT1 ISG15 HERC6 RETN | 0.893 | 0.884 | 0.902 | 0.917 | 0.858 | 0.977 | 0.932 | 0.863 | 1.000 |
| IFI27 OAS1 SIGLEC1 HERC6 SLC1A2 OLAH | 0.893 | 0.884 | 0.902 | 0.928 | 0.878 | 0.978 | 0.941 | 0.882 | 1 |
| IFI27 IFIT1 ISG15 HERC6 OLAH RETN | 0.893 | 0.884 | 0.901 | 0.920 | 0.864 | 0.977 | 0.940 | 0.881 | 0.999 |
| IFI27 IFI44L SIGLEC1 ISG15 SLC1A2 OLAH | 0.893 | 0.885 | 0.901 | 0.929 | 0.879 | 0.979 | 0.945 | 0.887 | 1 |
| RSAD2 IFI27 HERC6 IL1R2 OLAH RETN | 0.893 | 0.884 | 0.901 | 0.922 | 0.867 | 0.977 | 0.939 | 0.879 | 0.999 |
| IFIT1 IFI44L SIGLEC1 IL1R2 FAM20A MMP8 | 0.893 | 0.884 | 0.901 | 0.920 | 0.864 | 0.977 | 0.966 | 0.915 | 1 |
| IFI27 OAS1 ISG15 SLC1A2 OLAH FAM20A | 0.893 | 0.884 | 0.902 | 0.927 | 0.873 | 0.981 | 0.917 | 0.837 | 0.998 |
| RSAD2 IFI27 ISG15 OLAH FAM20A RETN | 0.893 | 0.884 | 0.902 | 0.919 | 0.859 | 0.980 | 0.933 | 0.863 | 1 |
| IFI27 ISG15 IL1R2 OLAH RETN MMP8 | 0.893 | 0.884 | 0.901 | 0.923 | 0.868 | 0.978 | 0.948 | 0.890 | 1 |
| RSAD2 IFI27 IFIT1 HERC6 IL1R2 OLAH | 0.893 | 0.884 | 0.901 | 0.925 | 0.875 | 0.975 | 0.939 | 0.884 | 0.995 |
| IFI27 IFI44L SLC1A2 IL1R2 FAM20A MMP8 | 0.893 | 0.884 | 0.901 | 0.929 | 0.882 | 0.976 | 0.926 | 0.839 | 1 |
| RSAD2 IFI27 IFI44L HERC6 IL1R2 OLAH | 0.893 | 0.884 | 0.901 | 0.926 | 0.877 | 0.975 | 0.936 | 0.878 | 0.993 |
| IFI27 SLC1A2 IL1R2 OLAH FAM20A RETN | 0.893 | 0.884 | 0.902 | 0.927 | 0.874 | 0.980 | 0.920 | 0.837 | 1 |
| RSAD2 IFI27 SIGLEC1 HERC6 IL1R2 RETN | 0.893 | 0.884 | 0.901 | 0.922 | 0.870 | 0.975 | 0.949 | 0.900 | 0.998 |
| RSAD2 IFI27 OAS1 IFIT1 IL1R2 FAM20A | 0.893 | 0.884 | 0.901 | 0.923 | 0.869 | 0.977 | 0.929 | 0.850 | 1 |
| IFI27 IFI44L SIGLEC1 OLAH RETN MMP8 | 0.893 | 0.884 | 0.902 | 0.920 | 0.862 | 0.978 | 0.959 | 0.908 | 1 |
| IFI27 SIGLEC1 ISG15 HERC6 IL1R2 OLAH | 0.893 | 0.884 | 0.901 | 0.924 | 0.874 | 0.975 | 0.943 | 0.891 | 0.996 |
| IFIT1 IFI44L SIGLEC1 HERC6 IL1R2 FAM20A | 0.893 | 0.884 | 0.901 | 0.918 | 0.861 | 0.976 | 0.952 | 0.895 | 1 |
| RSAD2 IFI27 IFI44L HERC6 OLAH MMP8 | 0.893 | 0.884 | 0.901 | 0.926 | 0.874 | 0.977 | 0.934 | 0.875 | 0.993 |
| RSAD2 IFI27 OAS1 SIGLEC1 HERC6 FAM20A | 0.893 | 0.883 | 0.902 | 0.918 | 0.858 | 0.977 | 0.940 | 0.868 | 1 |
| IFI27 OAS1 ISG15 HERC6 OLAH MMP8 | 0.893 | 0.884 | 0.901 | 0.923 | 0.871 | 0.975 | 0.937 | 0.879 | 0.995 |
| IFI27 ISG15 SLC1A2 OLAH FAM20A RETN | 0.893 | 0.884 | 0.901 | 0.926 | 0.872 | 0.980 | 0.916 | 0.834 | 0.998 |
| IFI27 OAS1 IFI44L OLAH FAM20A RETN | 0.893 | 0.883 | 0.902 | 0.920 | 0.860 | 0.981 | 0.938 | 0.870 | 1 |
| RSAD2 IFI27 IFIT1 SIGLEC1 HERC6 OLAH | 0.893 | 0.884 | 0.901 | 0.924 | 0.873 | 0.976 | 0.939 | 0.885 | 0.994 |
| RSAD2 IFI27 ISG15 IL1R2 FAM20A MMP8 | 0.893 | 0.884 | 0.901 | 0.926 | 0.875 | 0.978 | 0.928 | 0.843 | 1 |
| IFI27 OAS1 SIGLEC1 ISG15 HERC6 OLAH | 0.892 | 0.884 | 0.901 | 0.923 | 0.870 | 0.976 | 0.939 | 0.883 | 0.995 |
| RSAD2 IFI27 IFI44L HERC6 OLAH RETN | 0.892 | 0.884 | 0.901 | 0.923 | 0.869 | 0.977 | 0.935 | 0.874 | 0.996 |
| IFI27 OAS1 IFIT1 HERC6 IL1R2 RETN | 0.892 | 0.884 | 0.901 | 0.922 | 0.870 | 0.975 | 0.945 | 0.888 | 1 |
| RSAD2 IFI27 IFIT1 SIGLEC1 FAM20A RETN | 0.892 | 0.883 | 0.901 | 0.920 | 0.860 | 0.979 | 0.947 | 0.880 | 1 |
| OAS1 SIGLEC1 HERC6 IL1R2 FAM20A MMP8 | 0.892 | 0.884 | 0.901 | 0.918 | 0.861 | 0.975 | 0.964 | 0.910 | 1 |
| IFI27 IFIT1 SIGLEC1 ISG15 SLC1A2 OLAH | 0.892 | 0.884 | 0.901 | 0.929 | 0.879 | 0.979 | 0.938 | 0.878 | 0.998 |
| RSAD2 IFI27 SLC1A2 IL1R2 OLAH FAM20A | 0.892 | 0.884 | 0.901 | 0.927 | 0.873 | 0.981 | 0.921 | 0.838 | 1 |
| IFI27 OAS1 IFIT1 SIGLEC1 HERC6 IL1R2 | 0.892 | 0.885 | 0.900 | 0.922 | 0.876 | 0.969 | 0.949 | 0.902 | 0.996 |
| IFI27 SIGLEC1 ISG15 HERC6 OLAH MMP8 | 0.892 | 0.884 | 0.901 | 0.918 | 0.862 | 0.975 | 0.941 | 0.886 | 0.996 |
| IFI27 IFI44L SIGLEC1 FAM20A RETN MMP8 | 0.892 | 0.883 | 0.902 | 0.920 | 0.861 | 0.979 | 0.950 | 0.878 | 1 |
| RSAD2 IFI27 OAS1 IFI44L IL1R2 FAM20A | 0.892 | 0.884 | 0.901 | 0.923 | 0.869 | 0.977 | 0.932 | 0.852 | 1 |
| RSAD2 IFI27 OAS1 IFIT1 HERC6 OLAH | 0.892 | 0.884 | 0.901 | 0.922 | 0.870 | 0.975 | 0.934 | 0.874 | 0.993 |
| IFI27 OAS1 IFIT1 IL1R2 FAM20A RETN | 0.892 | 0.884 | 0.901 | 0.925 | 0.872 | 0.978 | 0.929 | 0.850 | 1 |
| IFI27 SLC1A2 OLAH FAM20A RETN MMP8 | 0.892 | 0.883 | 0.902 | 0.927 | 0.873 | 0.981 | 0.924 | 0.845 | 1 |
| IFI27 IFI44L HERC6 IL1R2 OLAH RETN | 0.892 | 0.884 | 0.901 | 0.923 | 0.868 | 0.977 | 0.943 | 0.888 | 0.999 |
| IFI27 HERC6 IL1R2 OLAH RETN MMP8 | 0.892 | 0.883 | 0.901 | 0.921 | 0.863 | 0.979 | 0.954 | 0.904 | 1 |
| IFI27 OAS1 IFI44L ISG15 HERC6 RETN | 0.892 | 0.883 | 0.901 | 0.917 | 0.857 | 0.977 | 0.935 | 0.868 | 1 |
| IFI27 OAS1 ISG15 HERC6 IL1R2 OLAH | 0.892 | 0.884 | 0.901 | 0.926 | 0.877 | 0.975 | 0.939 | 0.883 | 0.996 |
| IFI27 OAS1 HERC6 IL1R2 RETN MMP8 | 0.892 | 0.884 | 0.901 | 0.922 | 0.869 | 0.975 | 0.936 | 0.873 | 0.999 |
| IFI27 OAS1 IFIT1 IFI44L HERC6 OLAH | 0.892 | 0.884 | 0.901 | 0.926 | 0.874 | 0.977 | 0.937 | 0.880 | 0.994 |
| IFI27 SIGLEC1 HERC6 IL1R2 OLAH MMP8 | 0.892 | 0.884 | 0.900 | 0.923 | 0.872 | 0.974 | 0.949 | 0.900 | 0.998 |
| IFI27 IFI44L SIGLEC1 ISG15 OLAH MMP8 | 0.892 | 0.884 | 0.900 | 0.920 | 0.865 | 0.975 | 0.942 | 0.887 | 0.998 |
| IFI27 OAS1 IFI44L SLC1A2 OLAH FAM20A | 0.892 | 0.883 | 0.901 | 0.929 | 0.873 | 0.984 | 0.923 | 0.843 | 1 |
| IFI27 OAS1 IFI44L ISG15 IL1R2 FAM20A | 0.892 | 0.884 | 0.901 | 0.923 | 0.869 | 0.977 | 0.930 | 0.851 | 1 |
| IFI27 IFIT1 HERC6 IL1R2 OLAH RETN | 0.892 | 0.883 | 0.901 | 0.920 | 0.864 | 0.977 | 0.943 | 0.887 | 1.000 |
| RSAD2 IFI27 IFIT1 HERC6 OLAH MMP8 | 0.892 | 0.884 | 0.901 | 0.923 | 0.870 | 0.975 | 0.936 | 0.878 | 0.994 |
| IFI27 ISG15 HERC6 IL1R2 OLAH MMP8 | 0.892 | 0.884 | 0.900 | 0.924 | 0.874 | 0.974 | 0.942 | 0.888 | 0.997 |
| IFI27 IFIT1 SIGLEC1 IL1R2 OLAH MMP8 | 0.892 | 0.884 | 0.900 | 0.923 | 0.872 | 0.974 | 0.951 | 0.904 | 0.998 |
| RSAD2 IFI27 OAS1 OLAH FAM20A RETN | 0.892 | 0.883 | 0.901 | 0.919 | 0.858 | 0.980 | 0.937 | 0.869 | 1 |
| IFI27 SIGLEC1 HERC6 SLC1A2 IL1R2 FAM20A | 0.892 | 0.883 | 0.901 | 0.928 | 0.877 | 0.979 | 0.948 | 0.877 | 1 |
| RSAD2 IFI27 OAS1 SIGLEC1 HERC6 IL1R2 | 0.892 | 0.884 | 0.900 | 0.922 | 0.876 | 0.969 | 0.951 | 0.905 | 0.997 |
| IFI27 IFIT1 SIGLEC1 OLAH RETN MMP8 | 0.892 | 0.883 | 0.901 | 0.919 | 0.860 | 0.978 | 0.962 | 0.911 | 1 |
| IFI27 IFIT1 HERC6 IL1R2 OLAH MMP8 | 0.892 | 0.884 | 0.900 | 0.924 | 0.874 | 0.975 | 0.942 | 0.888 | 0.997 |
| RSAD2 IFI27 IFIT1 HERC6 FAM20A RETN | 0.892 | 0.883 | 0.901 | 0.922 | 0.864 | 0.979 | 0.933 | 0.853 | 1 |
| RSAD2 IFI27 IFI44L SLC1A2 OLAH FAM20A | 0.892 | 0.883 | 0.901 | 0.925 | 0.869 | 0.982 | 0.923 | 0.844 | 1 |
| IFI27 IFIT1 SIGLEC1 HERC6 IL1R2 RETN | 0.892 | 0.884 | 0.900 | 0.924 | 0.871 | 0.976 | 0.951 | 0.905 | 0.998 |
| IFI27 IFIT1 SLC1A2 IL1R2 OLAH FAM20A | 0.892 | 0.883 | 0.901 | 0.925 | 0.871 | 0.980 | 0.923 | 0.842 | 1 |
| RSAD2 IFI27 OAS1 SIGLEC1 FAM20A RETN | 0.892 | 0.882 | 0.901 | 0.918 | 0.857 | 0.979 | 0.949 | 0.883 | 1 |
| IFI27 OAS1 IFI44L SIGLEC1 HERC6 IL1R2 | 0.892 | 0.884 | 0.899 | 0.922 | 0.876 | 0.968 | 0.939 | 0.886 | 0.992 |
| RSAD2 IFI27 SIGLEC1 ISG15 HERC6 FAM20A | 0.892 | 0.883 | 0.901 | 0.918 | 0.858 | 0.978 | 0.941 | 0.869 | 1 |
| RSAD2 IFI27 OAS1 HERC6 IL1R2 RETN | 0.892 | 0.884 | 0.900 | 0.920 | 0.865 | 0.975 | 0.940 | 0.877 | 1 |
| OAS1 IFIT1 SIGLEC1 HERC6 IL1R2 FAM20A | 0.892 | 0.883 | 0.900 | 0.919 | 0.862 | 0.976 | 0.953 | 0.897 | 1 |
| IFIT1 SIGLEC1 ISG15 HERC6 IL1R2 FAM20A | 0.892 | 0.883 | 0.900 | 0.918 | 0.860 | 0.975 | 0.953 | 0.897 | 1 |
| RSAD2 IFI27 SIGLEC1 ISG15 HERC6 OLAH | 0.892 | 0.883 | 0.900 | 0.923 | 0.871 | 0.975 | 0.939 | 0.884 | 0.994 |
| IFI27 OAS1 SIGLEC1 IL1R2 OLAH MMP8 | 0.892 | 0.884 | 0.900 | 0.922 | 0.871 | 0.973 | 0.947 | 0.896 | 0.998 |
| RSAD2 IFI27 SIGLEC1 IL1R2 OLAH MMP8 | 0.892 | 0.884 | 0.900 | 0.922 | 0.872 | 0.973 | 0.949 | 0.900 | 0.998 |
| IFI27 SIGLEC1 ISG15 SLC1A2 IL1R2 OLAH | 0.892 | 0.884 | 0.900 | 0.930 | 0.881 | 0.980 | 0.939 | 0.880 | 0.998 |
| RSAD2 IFI27 ISG15 HERC6 IL1R2 OLAH | 0.892 | 0.884 | 0.900 | 0.924 | 0.873 | 0.974 | 0.937 | 0.880 | 0.994 |
| RSAD2 IFI27 IFI44L ISG15 IL1R2 FAM20A | 0.892 | 0.883 | 0.900 | 0.924 | 0.870 | 0.978 | 0.928 | 0.848 | 1 |
| IFI27 IFIT1 IFI44L HERC6 OLAH MMP8 | 0.892 | 0.883 | 0.900 | 0.924 | 0.871 | 0.977 | 0.936 | 0.877 | 0.994 |
| RSAD2 IFI27 IFIT1 OLAH FAM20A RETN | 0.892 | 0.883 | 0.901 | 0.918 | 0.857 | 0.979 | 0.935 | 0.864 | 1 |
| RSAD2 IFI27 IFI44L HERC6 FAM20A RETN | 0.892 | 0.882 | 0.901 | 0.924 | 0.867 | 0.982 | 0.932 | 0.851 | 1 |
| RSAD2 IFI27 OAS1 SLC1A2 OLAH FAM20A | 0.892 | 0.883 | 0.901 | 0.924 | 0.867 | 0.980 | 0.923 | 0.843 | 1 |
| IFI27 IFI44L HERC6 SLC1A2 OLAH FAM20A | 0.892 | 0.882 | 0.902 | 0.926 | 0.870 | 0.981 | 0.925 | 0.845 | 1 |
| IFI44L SIGLEC1 IL1R2 OLAH FAM20A MMP8 | 0.892 | 0.883 | 0.900 | 0.918 | 0.861 | 0.975 | 0.962 | 0.906 | 1 |
| IFI27 IFIT1 ISG15 HERC6 OLAH MMP8 | 0.892 | 0.883 | 0.900 | 0.921 | 0.866 | 0.975 | 0.936 | 0.877 | 0.995 |
| IFI27 OAS1 IFI44L HERC6 IL1R2 OLAH | 0.892 | 0.883 | 0.900 | 0.928 | 0.880 | 0.976 | 0.936 | 0.878 | 0.994 |
| IFI27 IFIT1 IFI44L ISG15 HERC6 OLAH | 0.892 | 0.883 | 0.900 | 0.924 | 0.871 | 0.977 | 0.935 | 0.876 | 0.993 |
| RSAD2 IFI27 IFIT1 SLC1A2 OLAH FAM20A | 0.892 | 0.883 | 0.901 | 0.923 | 0.865 | 0.980 | 0.927 | 0.851 | 1 |
| IFIT1 SIGLEC1 HERC6 IL1R2 OLAH FAM20A | 0.892 | 0.883 | 0.900 | 0.919 | 0.861 | 0.977 | 0.952 | 0.895 | 1 |
| IFI27 OAS1 ISG15 HERC6 IL1R2 RETN | 0.892 | 0.883 | 0.900 | 0.922 | 0.870 | 0.975 | 0.938 | 0.877 | 0.999 |
| IFI27 OAS1 IFI44L IL1R2 FAM20A RETN | 0.892 | 0.883 | 0.900 | 0.925 | 0.872 | 0.978 | 0.929 | 0.850 | 1 |
| IFI27 OAS1 SIGLEC1 SLC1A2 OLAH MMP8 | 0.892 | 0.883 | 0.900 | 0.927 | 0.875 | 0.978 | 0.945 | 0.888 | 1 |
| RSAD2 IFI27 ISG15 HERC6 OLAH RETN | 0.892 | 0.883 | 0.900 | 0.923 | 0.868 | 0.978 | 0.938 | 0.877 | 0.999 |
| IFI27 SIGLEC1 ISG15 HERC6 FAM20A RETN | 0.892 | 0.882 | 0.901 | 0.915 | 0.852 | 0.978 | 0.947 | 0.872 | 1 |
| RSAD2 SIGLEC1 IL1R2 OLAH FAM20A MMP8 | 0.892 | 0.883 | 0.900 | 0.919 | 0.863 | 0.976 | 0.964 | 0.911 | 1 |
| IFI27 SIGLEC1 ISG15 OLAH RETN MMP8 | 0.892 | 0.882 | 0.901 | 0.917 | 0.857 | 0.978 | 0.960 | 0.910 | 1 |
| IFI27 SIGLEC1 SLC1A2 OLAH FAM20A RETN | 0.892 | 0.882 | 0.901 | 0.924 | 0.865 | 0.982 | 0.943 | 0.876 | 1 |
| RSAD2 IFI27 OAS1 ISG15 HERC6 OLAH | 0.892 | 0.883 | 0.900 | 0.923 | 0.871 | 0.975 | 0.936 | 0.877 | 0.995 |
| RSAD2 IFIT1 SIGLEC1 IL1R2 FAM20A MMP8 | 0.892 | 0.883 | 0.900 | 0.922 | 0.865 | 0.978 | 0.967 | 0.916 | 1 |
| IFI27 IFIT1 ISG15 HERC6 IL1R2 OLAH | 0.892 | 0.883 | 0.900 | 0.924 | 0.874 | 0.974 | 0.942 | 0.888 | 0.997 |
| IFI27 OAS1 IFIT1 ISG15 HERC6 OLAH | 0.892 | 0.883 | 0.900 | 0.923 | 0.871 | 0.975 | 0.938 | 0.880 | 0.996 |
| RSAD2 IFI27 OAS1 IFI44L HERC6 OLAH | 0.891 | 0.883 | 0.900 | 0.926 | 0.875 | 0.977 | 0.937 | 0.879 | 0.995 |
| SIGLEC1 ISG15 IL1R2 OLAH FAM20A MMP8 | 0.891 | 0.883 | 0.900 | 0.916 | 0.857 | 0.974 | 0.964 | 0.909 | 1 |
| IFI27 IFIT1 SIGLEC1 ISG15 IL1R2 OLAH | 0.891 | 0.884 | 0.899 | 0.921 | 0.868 | 0.973 | 0.945 | 0.891 | 0.998 |
| IFI27 OAS1 HERC6 SLC1A2 OLAH MMP8 | 0.891 | 0.883 | 0.900 | 0.928 | 0.878 | 0.977 | 0.930 | 0.864 | 0.997 |
| IFI27 OAS1 IFIT1 SLC1A2 OLAH FAM20A | 0.891 | 0.882 | 0.901 | 0.924 | 0.868 | 0.981 | 0.926 | 0.850 | 1 |
| IFI27 IFI44L SIGLEC1 SLC1A2 OLAH MMP8 | 0.891 | 0.883 | 0.900 | 0.927 | 0.876 | 0.978 | 0.949 | 0.895 | 1 |
| IFI27 SIGLEC1 ISG15 HERC6 OLAH RETN | 0.891 | 0.882 | 0.900 | 0.917 | 0.858 | 0.976 | 0.945 | 0.888 | 1 |
| IFI27 IFI44L SIGLEC1 HERC6 IL1R2 MMP8 | 0.891 | 0.884 | 0.890 | 0.922 | 0.875 | 0.970 | 0.937 | 0.883 | 0.991 |
| IFI27 IFI44L IL1R2 OLAH RETN MMP8 | 0.891 | 0.882 | 0.900 | 0.923 | 0.868 | 0.978 | 0.951 | 0.897 | 1 |
| RSAD2 IFI27 OAS1 ISG15 HERC6 RETN | 0.891 | 0.882 | 0.900 | 0.919 | 0.860 | 0.979 | 0.937 | 0.869 | 1 |
| IFI44L SIGLEC1 ISG15 IL1R2 FAM20A MMP8 | 0.891 | 0.883 | 0.900 | 0.916 | 0.858 | 0.975 | 0.961 | 0.903 | 1 |
| IFI27 OAS1 IFI44L HERC6 IL1R2 RETN | 0.891 | 0.883 | 0.899 | 0.922 | 0.869 | 0.974 | 0.938 | 0.878 | 0.998 |
| IFI27 OAS1 HERC6 SLC1A2 OLAH RETN | 0.891 | 0.883 | 0.900 | 0.927 | 0.877 | 0.978 | 0.932 | 0.863 | 1 |
| IFI27 SIGLEC1 ISG15 HERC6 SLC1A2 OLAH | 0.891 | 0.882 | 0.900 | 0.930 | 0.879 | 0.980 | 0.939 | 0.880 | 0.998 |
| IFI27 OAS1 IFI44L SIGLEC1 FAM20A RETN | 0.891 | 0.882 | 0.900 | 0.919 | 0.859 | 0.979 | 0.943 | 0.873 | 1 |
| SIGLEC1 HERC6 IL1R2 OLAH FAM20A MMP8 | 0.891 | 0.882 | 0.900 | 0.918 | 0.859 | 0.976 | 0.964 | 0.909 | 1 |
| IFI27 IFI44L ISG15 HERC6 FAM20A RETN | 0.891 | 0.882 | 0.901 | 0.920 | 0.861 | 0.979 | 0.940 | 0.862 | 1 |
| IFI27 HERC6 SLC1A2 OLAH FAM20A RETN | 0.891 | 0.882 | 0.900 | 0.927 | 0.872 | 0.983 | 0.926 | 0.847 | 1 |
| IFI27 SIGLEC1 IL1R2 OLAH RETN MMP8 | 0.891 | 0.882 | 0.900 | 0.920 | 0.863 | 0.978 | 0.961 | 0.913 | 1 |
| IFI27 IFI44L SIGLEC1 IL1R2 OLAH RETN | 0.891 | 0.883 | 0.899 | 0.919 | 0.864 | 0.975 | 0.952 | 0.900 | 1 |
| IFI27 OAS1 IFIT1 SIGLEC1 IL1R2 OLAH | 0.891 | 0.883 | 0.899 | 0.921 | 0.869 | 0.973 | 0.942 | 0.888 | 0.996 |
| RSAD2 IFI27 IFI44L OLAH FAM20A RETN | 0.891 | 0.882 | 0.901 | 0.921 | 0.860 | 0.981 | 0.937 | 0.869 | 1 |
| IFI27 IFIT1 IFI44L SIGLEC1 IL1R2 OLAH | 0.891 | 0.883 | 0.899 | 0.922 | 0.870 | 0.973 | 0.948 | 0.897 | 0.998 |
| IFI27 OAS1 SLC1A2 IL1R2 OLAH FAM20A | 0.891 | 0.883 | 0.900 | 0.928 | 0.875 | 0.981 | 0.920 | 0.836 | 1 |
| IFI27 OAS1 SIGLEC1 OLAH RETN MMP8 | 0.891 | 0.882 | 0.900 | 0.920 | 0.862 | 0.979 | 0.962 | 0.909 | 1 |
| IFI27 OAS1 IFI44L ISG15 HERC6 OLAH | 0.891 | 0.882 | 0.900 | 0.924 | 0.873 | 0.976 | 0.935 | 0.876 | 0.994 |
| IFI27 OAS1 IFI44L SIGLEC1 IL1R2 OLAH | 0.891 | 0.883 | 0.899 | 0.923 | 0.872 | 0.974 | 0.947 | 0.896 | 0.997 |
| RSAD2 IFI27 ISG15 HERC6 OLAH MMP8 | 0.891 | 0.883 | 0.900 | 0.923 | 0.871 | 0.976 | 0.936 | 0.877 | 0.995 |
| IFI27 OAS1 IFIT1 ISG15 IL1R2 FAM20A | 0.891 | 0.882 | 0.900 | 0.924 | 0.871 | 0.978 | 0.929 | 0.850 | 1 |
| IFI27 IFI44L ISG15 IL1R2 OLAH MMP8 | 0.891 | 0.883 | 0.899 | 0.922 | 0.871 | 0.974 | 0.939 | 0.882 | 0.996 |
| RSAD2 OAS1 SIGLEC1 IL1R2 FAM20A MMP8 | 0.891 | 0.882 | 0.900 | 0.919 | 0.862 | 0.976 | 0.962 | 0.905 | 1 |
| IFI27 ISG15 HERC6 IL1R2 OLAH RETN | 0.891 | 0.882 | 0.900 | 0.921 | 0.865 | 0.977 | 0.943 | 0.887 | 1 |
| IFIT1 SIGLEC1 ISG15 IL1R2 FAM20A MMP8 | 0.891 | 0.883 | 0.900 | 0.919 | 0.861 | 0.976 | 0.966 | 0.915 | 1 |
| IFI27 IFIT1 IFI44L SIGLEC1 HERC6 RETN | 0.891 | 0.882 | 0.900 | 0.911 | 0.850 | 0.973 | 0.941 | 0.887 | 0.995 |
| IFI27 IFIT1 IFI44L SIGLEC1 FAM20A MMP8 | 0.891 | 0.882 | 0.900 | 0.913 | 0.848 | 0.978 | 0.946 | 0.877 | 1 |
| RSAD2 IFI27 SIGLEC1 HERC6 SLC1A2 OLAH | 0.891 | 0.882 | 0.900 | 0.928 | 0.877 | 0.978 | 0.937 | 0.878 | 0.996 |
| IFI27 OAS1 IFIT1 IFI44L SIGLEC1 FAM20A | 0.891 | 0.882 | 0.900 | 0.913 | 0.848 | 0.979 | 0.947 | 0.878 | 1 |
| RSAD2 IFI27 SIGLEC1 ISG15 FAM20A RETN | 0.891 | 0.881 | 0.900 | 0.918 | 0.858 | 0.979 | 0.948 | 0.881 | 1 |
| IFI27 OAS1 ISG15 IL1R2 FAM20A RETN | 0.891 | 0.882 | 0.899 | 0.926 | 0.873 | 0.978 | 0.930 | 0.851 | 1 |
| IFI27 OAS1 IFIT1 SIGLEC1 OLAH RETN | 0.891 | 0.882 | 0.899 | 0.916 | 0.858 | 0.974 | 0.949 | 0.892 | 1 |
| IFIT1 SIGLEC1 HERC6 OLAH FAM20A MMP8 | 0.891 | 0.881 | 0.900 | 0.918 | 0.860 | 0.976 | 0.954 | 0.900 | 1 |
| IFI27 IFI44L SIGLEC1 IL1R2 RETN MMP8 | 0.891 | 0.883 | 0.899 | 0.923 | 0.871 | 0.976 | 0.961 | 0.920 | 1 |
| RSAD2 IFI27 IFIT1 ISG15 IL1R2 FAM20A | 0.891 | 0.882 | 0.899 | 0.923 | 0.870 | 0.977 | 0.929 | 0.850 | 1 |
| RSAD2 IFIT1 SIGLEC1 HERC6 IL1R2 FAM20A | 0.891 | 0.882 | 0.899 | 0.918 | 0.860 | 0.976 | 0.954 | 0.898 | 1 |
| IFI27 IFIT1 IL1R2 OLAH RETN MMP8 | 0.891 | 0.882 | 0.900 | 0.921 | 0.864 | 0.977 | 0.952 | 0.900 | 1 |
| RSAD2 IFI27 OAS1 ISG15 IL1R2 FAM20A | 0.891 | 0.882 | 0.899 | 0.925 | 0.872 | 0.978 | 0.932 | 0.852 | 1 |
| IFI27 IFI44L SIGLEC1 SLC1A2 IL1R2 OLAH | 0.891 | 0.882 | 0.899 | 0.927 | 0.877 | 0.978 | 0.943 | 0.888 | 0.999 |
| RSAD2 IFI27 IFIT1 IL1R2 FAM20A RETN | 0.891 | 0.882 | 0.899 | 0.924 | 0.871 | 0.978 | 0.929 | 0.850 | 1 |
| RSAD2 IFI27 SIGLEC1 HERC6 FAM20A MMP8 | 0.891 | 0.881 | 0.900 | 0.918 | 0.858 | 0.978 | 0.940 | 0.868 | 1 |
| SIGLEC1 ISG15 HERC6 IL1R2 FAM20A MMP8 | 0.891 | 0.882 | 0.900 | 0.915 | 0.855 | 0.974 | 0.962 | 0.903 | 1 |
| IFI27 OAS1 SIGLEC1 ISG15 HERC6 FAM20A | 0.891 | 0.881 | 0.900 | 0.914 | 0.853 | 0.976 | 0.942 | 0.870 | 1 |
| IFI27 OAS1 SIGLEC1 IL1R2 RETN MMP8 | 0.891 | 0.882 | 0.899 | 0.923 | 0.871 | 0.976 | 0.963 | 0.918 | 1 |
| RSAD2 IFI27 IFI44L SIGLEC1 HERC6 FAM20A | 0.891 | 0.882 | 0.899 | 0.918 | 0.858 | 0.977 | 0.943 | 0.871 | 1 |
| IFI27 IFIT1 SIGLEC1 SLC1A2 OLAH MMP8 | 0.891 | 0.882 | 0.900 | 0.928 | 0.877 | 0.979 | 0.942 | 0.884 | 1 |
| IFI27 IFIT1 ISG15 IL1R2 FAM20A RETN | 0.891 | 0.882 | 0.899 | 0.926 | 0.873 | 0.979 | 0.929 | 0.850 | 1 |
| IFI27 IFI44L SIGLEC1 SLC1A2 OLAH RETN | 0.891 | 0.882 | 0.899 | 0.926 | 0.875 | 0.978 | 0.951 | 0.895 | 1 |
| RSAD2 IFI27 SIGLEC1 ISG15 SLC1A2 OLAH | 0.891 | 0.882 | 0.899 | 0.929 | 0.878 | 0.980 | 0.938 | 0.878 | 0.998 |
| IFI27 IFIT1 SIGLEC1 ISG15 OLAH MMP8 | 0.891 | 0.882 | 0.899 | 0.919 | 0.862 | 0.975 | 0.941 | 0.885 | 0.998 |
| IFI27 OAS1 ISG15 HERC6 SLC1A2 OLAH | 0.891 | 0.882 | 0.900 | 0.929 | 0.879 | 0.978 | 0.924 | 0.855 | 0.993 |
| IFI27 OAS1 IFIT1 OLAH RETN MMP8 | 0.891 | 0.881 | 0.900 | 0.918 | 0.858 | 0.978 | 0.952 | 0.896 | 1 |
| IFI27 IFIT1 IFI44L HERC6 IL1R2 OLAH | 0.891 | 0.882 | 0.899 | 0.926 | 0.876 | 0.976 | 0.936 | 0.879 | 0.993 |
| RSAD2 IFI27 SIGLEC1 SLC1A2 OLAH MMP8 | 0.891 | 0.882 | 0.899 | 0.927 | 0.876 | 0.979 | 0.943 | 0.886 | 1 |
| IFI27 IFIT1 ISG15 SLC1A2 OLAH MMP8 | 0.891 | 0.882 | 0.899 | 0.926 | 0.874 | 0.978 | 0.929 | 0.862 | 0.997 |
| IFI27 ISG15 SLC1A2 IL1R2 OLAH MMP8 | 0.891 | 0.883 | 0.899 | 0.927 | 0.878 | 0.976 | 0.925 | 0.854 | 0.996 |
| RSAD2 IFI27 IFI44L SIGLEC1 FAM20A RETN | 0.891 | 0.881 | 0.900 | 0.918 | 0.858 | 0.979 | 0.948 | 0.881 | 1 |
| IFI27 OAS1 IL1R2 OLAH RETN MMP8 | 0.891 | 0.882 | 0.899 | 0.922 | 0.866 | 0.977 | 0.953 | 0.902 | 1 |
| IFI27 IFI44L SIGLEC1 ISG15 HERC6 IL1R2 | 0.890 | 0.883 | 0.898 | 0.923 | 0.876 | 0.970 | 0.938 | 0.885 | 0.991 |
| RSAD2 IFI27 IFIT1 IFI44L HERC6 OLAH | 0.890 | 0.882 | 0.899 | 0.924 | 0.872 | 0.976 | 0.935 | 0.876 | 0.993 |
| IFIT1 IFI44L SIGLEC1 HERC6 OLAH FAM20A | 0.890 | 0.881 | 0.900 | 0.917 | 0.857 | 0.977 | 0.946 | 0.887 | 1 |
| RSAD2 IFI27 IFI44L SIGLEC1 IL1R2 OLAH | 0.890 | 0.883 | 0.898 | 0.924 | 0.873 | 0.975 | 0.950 | 0.902 | 0.998 |
| IFI27 IFI44L SIGLEC1 ISG15 OLAH RETN | 0.890 | 0.882 | 0.899 | 0.919 | 0.862 | 0.975 | 0.947 | 0.888 | 1 |
| RSAD2 IFI27 SIGLEC1 HERC6 IL1R2 MMP8 | 0.890 | 0.883 | 0.898 | 0.921 | 0.873 | 0.968 | 0.949 | 0.902 | 0.996 |
| IFI27 IFIT1 IFI44L SIGLEC1 HERC6 IL1R2 | 0.890 | 0.883 | 0.898 | 0.922 | 0.874 | 0.970 | 0.941 | 0.890 | 0.993 |
| IFI27 OAS1 SIGLEC1 ISG15 SLC1A2 OLAH | 0.890 | 0.882 | 0.899 | 0.928 | 0.877 | 0.979 | 0.939 | 0.880 | 0.998 |
| RSAD2 IFI27 OAS1 IL1R2 FAM20A RETN | 0.890 | 0.882 | 0.899 | 0.925 | 0.873 | 0.978 | 0.929 | 0.850 | 1 |
| IFI27 OAS1 HERC6 FAM20A RETN MMP8 | 0.890 | 0.881 | 0.900 | 0.923 | 0.865 | 0.981 | 0.934 | 0.853 | 1 |
| IFI27 SIGLEC1 SLC1A2 IL1R2 FAM20A RETN | 0.890 | 0.881 | 0.900 | 0.929 | 0.878 | 0.980 | 0.948 | 0.877 | 1 |
| IFI27 OAS1 IFIT1 HERC6 SLC1A2 OLAH | 0.890 | 0.881 | 0.900 | 0.928 | 0.878 | 0.978 | 0.933 | 0.867 | 0.998 |
| IFI27 OAS1 HERC6 SLC1A2 IL1R2 FAM20A | 0.890 | 0.881 | 0.899 | 0.930 | 0.884 | 0.977 | 0.923 | 0.835 | 1 |
| IFI27 OAS1 IFIT1 SIGLEC1 ISG15 FAM20A | 0.890 | 0.881 | 0.899 | 0.916 | 0.853 | 0.980 | 0.942 | 0.870 | 1 |
| RSAD2 IFI27 HERC6 FAM20A RETN MMP8 | 0.890 | 0.881 | 0.900 | 0.923 | 0.865 | 0.980 | 0.934 | 0.854 | 1 |
| RSAD2 IFI27 IFIT1 SIGLEC1 HERC6 RETN | 0.890 | 0.882 | 0.899 | 0.915 | 0.855 | 0.974 | 0.941 | 0.886 | 0.997 |
| RSAD2 IFI27 IFI44L ISG15 HERC6 OLAH | 0.890 | 0.882 | 0.899 | 0.923 | 0.871 | 0.975 | 0.934 | 0.875 | 0.993 |
| IFI27 OAS1 SIGLEC1 HERC6 FAM20A MMP8 | 0.890 | 0.881 | 0.900 | 0.915 | 0.855 | 0.976 | 0.940 | 0.868 | 1 |
| IFI27 SIGLEC1 HERC6 FAM20A RETN MMP8 | 0.890 | 0.880 | 0.901 | 0.918 | 0.855 | 0.981 | 0.953 | 0.881 | 1 |
| IFI27 OAS1 SIGLEC1 SLC1A2 IL1R2 OLAH | 0.890 | 0.882 | 0.899 | 0.927 | 0.877 | 0.977 | 0.947 | 0.891 | 1 |
| IFI27 SIGLEC1 ISG15 FAM20A RETN MMP8 | 0.890 | 0.880 | 0.900 | 0.917 | 0.853 | 0.981 | 0.951 | 0.878 | 1 |
| RSAD2 IFI27 OAS1 IFI44L HERC6 RETN | 0.890 | 0.881 | 0.899 | 0.915 | 0.853 | 0.977 | 0.934 | 0.861 | 1 |
| IFI27 IFI44L ISG15 OLAH RETN MMP8 | 0.890 | 0.881 | 0.899 | 0.919 | 0.860 | 0.978 | 0.950 | 0.894 | 1 |
| IFI27 IFI44L HERC6 IL1R2 RETN MMP8 | 0.890 | 0.882 | 0.899 | 0.922 | 0.868 | 0.975 | 0.933 | 0.869 | 0.996 |
| IFI27 OAS1 HERC6 SLC1A2 IL1R2 OLAH | 0.890 | 0.881 | 0.899 | 0.927 | 0.877 | 0.976 | 0.929 | 0.862 | 0.997 |
| IFI27 IFI44L SLC1A2 OLAH FAM20A RETN | 0.890 | 0.881 | 0.899 | 0.924 | 0.867 | 0.982 | 0.924 | 0.844 | 1 |
| IFI27 IFIT1 SIGLEC1 ISG15 FAM20A MMP8 | 0.890 | 0.881 | 0.899 | 0.916 | 0.853 | 0.980 | 0.942 | 0.871 | 1 |
| RSAD2 IFI27 IL1R2 OLAH RETN MMP8 | 0.890 | 0.881 | 0.899 | 0.921 | 0.865 | 0.977 | 0.953 | 0.900 | 1 |
| RSAD2 IFI27 OAS1 SIGLEC1 IL1R2 OLAH | 0.890 | 0.882 | 0.898 | 0.920 | 0.868 | 0.973 | 0.943 | 0.890 | 0.997 |
| IFI27 OAS1 IFIT1 SIGLEC1 OLAH MMP8 | 0.890 | 0.882 | 0.899 | 0.919 | 0.864 | 0.974 | 0.945 | 0.890 | 0.999 |
| RSAD2 IFI27 SIGLEC1 OLAH RETN MMP8 | 0.890 | 0.881 | 0.899 | 0.920 | 0.862 | 0.979 | 0.961 | 0.908 | 1 |
| RSAD2 IFI27 IFI44L IL1R2 FAM20A RETN | 0.890 | 0.881 | 0.899 | 0.924 | 0.870 | 0.978 | 0.928 | 0.848 | 1 |
| IFI27 IFIT1 SIGLEC1 IL1R2 OLAH RETN | 0.890 | 0.882 | 0.898 | 0.919 | 0.863 | 0.975 | 0.948 | 0.892 | 1 |
| RSAD2 IFI27 SIGLEC1 SLC1A2 IL1R2 OLAH | 0.890 | 0.881 | 0.899 | 0.928 | 0.878 | 0.977 | 0.942 | 0.885 | 0.999 |
| RSAD2 IFI27 HERC6 SLC1A2 OLAH MMP8 | 0.890 | 0.881 | 0.899 | 0.927 | 0.876 | 0.977 | 0.927 | 0.860 | 0.994 |
| IFI27 IFIT1 SIGLEC1 HERC6 SLC1A2 OLAH | 0.890 | 0.881 | 0.899 | 0.930 | 0.879 | 0.980 | 0.941 | 0.886 | 0.996 |
| IFI27 IFIT1 IFI44L SIGLEC1 ISG15 OLAH | 0.890 | 0.882 | 0.898 | 0.919 | 0.863 | 0.975 | 0.940 | 0.882 | 0.998 |
| RSAD2 IFI27 HERC6 SLC1A2 IL1R2 FAM20A | 0.890 | 0.881 | 0.899 | 0.930 | 0.883 | 0.977 | 0.925 | 0.838 | 1 |
| IFI27 IFIT1 SLC1A2 OLAH FAM20A RETN | 0.890 | 0.881 | 0.899 | 0.924 | 0.868 | 0.981 | 0.926 | 0.849 | 1 |
| RSAD2 IFI27 IFIT1 ISG15 HERC6 OLAH | 0.890 | 0.881 | 0.899 | 0.923 | 0.870 | 0.975 | 0.935 | 0.876 | 0.994 |
| IFI27 IFI44L ISG15 SLC1A2 OLAH MMP8 | 0.890 | 0.881 | 0.899 | 0.926 | 0.874 | 0.978 | 0.925 | 0.855 | 0.995 |
| IFI27 IFI44L SIGLEC1 ISG15 IL1R2 RETN | 0.890 | 0.882 | 0.898 | 0.921 | 0.869 | 0.974 | 0.950 | 0.899 | 1 |
| RSAD2 IFI27 IFI44L SIGLEC1 HERC6 IL1R2 | 0.890 | 0.882 | 0.898 | 0.923 | 0.874 | 0.971 | 0.940 | 0.888 | 0.992 |
| IFI27 SIGLEC1 ISG15 SLC1A2 OLAH RETN | 0.890 | 0.881 | 0.899 | 0.927 | 0.876 | 0.978 | 0.941 | 0.881 | 1 |
| IFI27 IFIT1 HERC6 FAM20A RETN MMP8 | 0.890 | 0.880 | 0.900 | 0.922 | 0.863 | 0.981 | 0.933 | 0.851 | 1 |
| RSAD2 IFI27 ISG15 IL1R2 FAM20A RETN | 0.890 | 0.881 | 0.899 | 0.925 | 0.872 | 0.977 | 0.928 | 0.849 | 1 |
| IFIT1 SIGLEC1 HERC6 FAM20A RETN MMP8 | 0.890 | 0.881 | 0.899 | 0.917 | 0.856 | 0.978 | 0.945 | 0.878 | 1 |
| IFI27 SIGLEC1 SLC1A2 IL1R2 OLAH MMP8 | 0.890 | 0.881 | 0.898 | 0.927 | 0.877 | 0.977 | 0.947 | 0.894 | 0.999 |
| IFI27 IFIT1 IFI44L SIGLEC1 OLAH RETN | 0.890 | 0.881 | 0.898 | 0.916 | 0.857 | 0.975 | 0.948 | 0.888 | 1 |
| IFI27 OAS1 IFIT1 IL1R2 OLAH MMP8 | 0.890 | 0.882 | 0.898 | 0.922 | 0.870 | 0.973 | 0.943 | 0.890 | 0.997 |
| IFI27 IFIT1 SLC1A2 IL1R2 FAM20A MMP8 | 0.890 | 0.881 | 0.899 | 0.927 | 0.878 | 0.976 | 0.929 | 0.845 | 1 |
| RSAD2 IFI27 SLC1A2 OLAH FAM20A RETN | 0.890 | 0.881 | 0.899 | 0.924 | 0.868 | 0.981 | 0.922 | 0.841 | 1 |
| IFI27 IFIT1 SIGLEC1 ISG15 HERC6 RETN | 0.890 | 0.881 | 0.899 | 0.910 | 0.848 | 0.972 | 0.949 | 0.897 | 1 |
| OAS1 SIGLEC1 IL1R2 OLAH FAM20A MMP8 | 0.890 | 0.881 | 0.899 | 0.918 | 0.861 | 0.975 | 0.964 | 0.911 | 1 |
| IFI27 SIGLEC1 ISG15 IL1R2 OLAH RETN | 0.890 | 0.881 | 0.898 | 0.919 | 0.863 | 0.975 | 0.947 | 0.891 | 1 |
| IFI27 OAS1 SIGLEC1 HERC6 SLC1A2 RETN | 0.890 | 0.881 | 0.899 | 0.922 | 0.867 | 0.976 | 0.947 | 0.885 | 1 |
| IFI27 OAS1 SIGLEC1 IL1R2 OLAH RETN | 0.890 | 0.881 | 0.898 | 0.919 | 0.863 | 0.974 | 0.952 | 0.898 | 1 |
| RSAD2 IFI27 IFIT1 IFI44L SIGLEC1 FAM20A | 0.890 | 0.881 | 0.899 | 0.915 | 0.850 | 0.980 | 0.948 | 0.880 | 1 |
| IFI27 OAS1 SIGLEC1 ISG15 IL1R2 OLAH | 0.890 | 0.882 | 0.898 | 0.921 | 0.869 | 0.973 | 0.943 | 0.889 | 0.998 |
| IFI27 IFIT1 SIGLEC1 SLC1A2 IL1R2 OLAH | 0.890 | 0.881 | 0.898 | 0.928 | 0.878 | 0.978 | 0.946 | 0.892 | 0.999 |
| IFI27 IFI44L SLC1A2 IL1R2 OLAH MMP8 | 0.890 | 0.881 | 0.898 | 0.927 | 0.878 | 0.977 | 0.934 | 0.869 | 0.998 |
| RSAD2 IFI27 IFIT1 SIGLEC1 IL1R2 OLAH | 0.890 | 0.882 | 0.898 | 0.921 | 0.869 | 0.973 | 0.942 | 0.888 | 0.996 |
| IFI27 OAS1 IFIT1 SIGLEC1 SLC1A2 OLAH | 0.890 | 0.881 | 0.898 | 0.927 | 0.876 | 0.979 | 0.945 | 0.888 | 1 |
| IFI27 IFIT1 IFI44L SIGLEC1 OLAH MMP8 | 0.890 | 0.881 | 0.898 | 0.919 | 0.863 | 0.976 | 0.949 | 0.898 | 1.000 |
| RSAD2 IFI27 HERC6 SLC1A2 IL1R2 OLAH | 0.890 | 0.881 | 0.898 | 0.927 | 0.877 | 0.976 | 0.928 | 0.862 | 0.994 |
| OAS1 IFI44L SIGLEC1 IL1R2 FAM20A MMP8 | 0.890 | 0.881 | 0.898 | 0.918 | 0.861 | 0.975 | 0.963 | 0.908 | 1 |
| IFI27 OAS1 ISG15 HERC6 RETN MMP8 | 0.890 | 0.881 | 0.899 | 0.917 | 0.858 | 0.976 | 0.935 | 0.868 | 1 |
| RSAD2 IFI27 OAS1 IFIT1 HERC6 RETN | 0.890 | 0.880 | 0.899 | 0.916 | 0.854 | 0.978 | 0.935 | 0.862 | 1 |
| IFI27 OAS1 IFI44L SIGLEC1 ISG15 OLAH | 0.890 | 0.881 | 0.898 | 0.919 | 0.863 | 0.975 | 0.941 | 0.887 | 0.996 |
| RSAD2 IFI27 HERC6 IL1R2 RETN MMP8 | 0.890 | 0.881 | 0.898 | 0.921 | 0.868 | 0.974 | 0.938 | 0.878 | 0.998 |
| IFI27 IFIT1 IFI44L SIGLEC1 IL1R2 RETN | 0.890 | 0.882 | 0.898 | 0.921 | 0.868 | 0.974 | 0.949 | 0.897 | 1 |
| IFI27 IFIT1 ISG15 OLAH RETN MMP8 | 0.890 | 0.880 | 0.899 | 0.918 | 0.858 | 0.978 | 0.951 | 0.895 | 1 |
| IFI27 IFIT1 IFI44L IL1R2 OLAH MMP8 | 0.890 | 0.881 | 0.898 | 0.922 | 0.870 | 0.973 | 0.942 | 0.889 | 0.995 |
| IFI27 IFIT1 SIGLEC1 IL1R2 RETN MMP8 | 0.890 | 0.881 | 0.898 | 0.921 | 0.867 | 0.975 | 0.963 | 0.923 | 1 |
| RSAD2 IFI27 SIGLEC1 ISG15 IL1R2 OLAH | 0.890 | 0.881 | 0.898 | 0.920 | 0.868 | 0.973 | 0.945 | 0.892 | 0.998 |
| IFI27 OAS1 SLC1A2 OLAH FAM20A RETN | 0.890 | 0.881 | 0.899 | 0.925 | 0.870 | 0.981 | 0.922 | 0.841 | 1 |
| IFIT1 SIGLEC1 IL1R2 FAM20A RETN MMP8 | 0.890 | 0.881 | 0.898 | 0.920 | 0.864 | 0.977 | 0.968 | 0.918 | 1 |
| IFI27 OAS1 IFI44L SIGLEC1 OLAH MMP8 | 0.890 | 0.881 | 0.898 | 0.920 | 0.864 | 0.976 | 0.949 | 0.898 | 1.000 |
| IFI27 OAS1 IFI44L SIGLEC1 HERC6 FAM20A | 0.890 | 0.881 | 0.898 | 0.915 | 0.855 | 0.975 | 0.946 | 0.872 | 1 |
| IFI27 IFIT1 SIGLEC1 ISG15 OLAH RETN | 0.890 | 0.881 | 0.898 | 0.917 | 0.860 | 0.975 | 0.946 | 0.888 | 1 |
| IFI27 OAS1 SIGLEC1 ISG15 HERC6 IL1R2 | 0.890 | 0.882 | 0.897 | 0.921 | 0.874 | 0.968 | 0.950 | 0.904 | 0.996 |
| IFI27 IFIT1 ISG15 IL1R2 OLAH MMP8 | 0.890 | 0.881 | 0.898 | 0.921 | 0.870 | 0.973 | 0.945 | 0.892 | 0.997 |
| RSAD2 IFI27 OAS1 IL1R2 OLAH MMP8 | 0.890 | 0.881 | 0.898 | 0.920 | 0.868 | 0.973 | 0.943 | 0.890 | 0.996 |
| IFI27 OAS1 SIGLEC1 ISG15 OLAH MMP8 | 0.889 | 0.881 | 0.898 | 0.918 | 0.861 | 0.974 | 0.943 | 0.887 | 1.000 |
| IFI27 SIGLEC1 HERC6 SLC1A2 OLAH MMP8 | 0.889 | 0.880 | 0.899 | 0.927 | 0.876 | 0.979 | 0.941 | 0.883 | 0.999 |
| IFI27 SIGLEC1 HERC6 IL1R2 RETN MMP8 | 0.889 | 0.881 | 0.898 | 0.922 | 0.868 | 0.977 | 0.963 | 0.923 | 1 |
| IFI27 IFIT1 SIGLEC1 SLC1A2 FAM20A RETN | 0.889 | 0.880 | 0.899 | 0.924 | 0.868 | 0.980 | 0.941 | 0.865 | 1 |
| RSAD2 IFI27 IFI44L SIGLEC1 HERC6 RETN | 0.889 | 0.881 | 0.898 | 0.916 | 0.857 | 0.975 | 0.942 | 0.887 | 0.997 |
| RSAD2 IFI27 OAS1 OLAH RETN MMP8 | 0.889 | 0.880 | 0.899 | 0.919 | 0.860 | 0.979 | 0.951 | 0.894 | 1 |
| IFI27 IFIT1 SIGLEC1 SLC1A2 OLAH RETN | 0.889 | 0.880 | 0.898 | 0.927 | 0.875 | 0.979 | 0.949 | 0.892 | 1 |
| IFI27 OAS1 IFIT1 IFI44L SIGLEC1 OLAH | 0.889 | 0.881 | 0.897 | 0.920 | 0.863 | 0.976 | 0.949 | 0.898 | 1.000 |
| RSAD2 IFI27 IFIT1 IL1R2 OLAH MMP8 | 0.889 | 0.881 | 0.898 | 0.922 | 0.870 | 0.974 | 0.946 | 0.894 | 0.997 |
| RSAD2 IFI27 IFIT1 SIGLEC1 HERC6 IL1R2 | 0.889 | 0.882 | 0.897 | 0.922 | 0.874 | 0.969 | 0.946 | 0.897 | 0.995 |
| RSAD2 IFI27 OAS1 SIGLEC1 SLC1A2 OLAH | 0.889 | 0.881 | 0.898 | 0.928 | 0.876 | 0.980 | 0.948 | 0.892 | 1 |
| RSAD2 IFI27 OAS1 HERC6 SLC1A2 OLAH | 0.889 | 0.880 | 0.898 | 0.929 | 0.879 | 0.979 | 0.932 | 0.864 | 0.999 |
| IFI27 OAS1 IFIT1 SIGLEC1 ISG15 OLAH | 0.889 | 0.881 | 0.898 | 0.918 | 0.862 | 0.974 | 0.941 | 0.885 | 0.998 |
| RSAD2 IFI27 IFIT1 SIGLEC1 ISG15 FAM20A | 0.889 | 0.880 | 0.898 | 0.916 | 0.852 | 0.980 | 0.941 | 0.870 | 1 |
| RSAD2 IFI27 OAS1 IFIT1 SIGLEC1 FAM20A | 0.889 | 0.880 | 0.899 | 0.915 | 0.850 | 0.979 | 0.947 | 0.877 | 1 |
| RSAD2 IFI27 SIGLEC1 IL1R2 RETN MMP8 | 0.889 | 0.881 | 0.898 | 0.922 | 0.868 | 0.975 | 0.962 | 0.921 | 1 |
| IFI27 IFI44L SIGLEC1 ISG15 IL1R2 MMP8 | 0.889 | 0.882 | 0.896 | 0.918 | 0.869 | 0.967 | 0.942 | 0.891 | 0.994 |
| RSAD2 IFI27 IFIT1 HERC6 IL1R2 RETN | 0.889 | 0.881 | 0.897 | 0.920 | 0.867 | 0.973 | 0.941 | 0.886 | 0.996 |
| IFI27 IFI44L ISG15 HERC6 IL1R2 RETN | 0.889 | 0.881 | 0.897 | 0.921 | 0.868 | 0.974 | 0.936 | 0.876 | 0.995 |
| RSAD2 IFI27 HERC6 SLC1A2 OLAH RETN | 0.889 | 0.880 | 0.898 | 0.926 | 0.876 | 0.977 | 0.935 | 0.870 | 1 |
| IFI27 OAS1 SIGLEC1 SLC1A2 OLAH RETN | 0.889 | 0.880 | 0.898 | 0.925 | 0.873 | 0.978 | 0.948 | 0.886 | 1 |
| IFI27 HERC6 SLC1A2 IL1R2 FAM20A RETN | 0.889 | 0.880 | 0.898 | 0.933 | 0.885 | 0.981 | 0.925 | 0.840 | 1 |
| IFI27 IFIT1 ISG15 HERC6 SLC1A2 OLAH | 0.889 | 0.880 | 0.898 | 0.928 | 0.877 | 0.978 | 0.928 | 0.861 | 0.995 |
| RSAD2 IFI27 SIGLEC1 ISG15 HERC6 RETN | 0.889 | 0.880 | 0.898 | 0.918 | 0.860 | 0.976 | 0.951 | 0.901 | 1 |
| IFIT1 SIGLEC1 HERC6 IL1R2 FAM20A RETN | 0.889 | 0.880 | 0.898 | 0.919 | 0.861 | 0.976 | 0.953 | 0.897 | 1 |
| IFI27 OAS1 IFIT1 IFI44L HERC6 RETN | 0.889 | 0.880 | 0.898 | 0.916 | 0.855 | 0.977 | 0.935 | 0.865 | 1 |
| IFI27 OAS1 IFI44L SIGLEC1 OLAH RETN | 0.889 | 0.881 | 0.897 | 0.916 | 0.857 | 0.975 | 0.948 | 0.888 | 1 |
| RSAD2 IFI44L SIGLEC1 IL1R2 FAM20A MMP8 | 0.889 | 0.880 | 0.898 | 0.919 | 0.862 | 0.975 | 0.963 | 0.908 | 1 |
| IFI27 IFI44L SLC1A2 IL1R2 FAM20A RETN | 0.889 | 0.880 | 0.898 | 0.926 | 0.875 | 0.977 | 0.925 | 0.840 | 1 |
| IFI27 ISG15 HERC6 SLC1A2 OLAH MMP8 | 0.889 | 0.880 | 0.898 | 0.928 | 0.877 | 0.978 | 0.926 | 0.859 | 0.993 |
| IFI27 SIGLEC1 ISG15 IL1R2 RETN MMP8 | 0.889 | 0.881 | 0.897 | 0.920 | 0.866 | 0.975 | 0.965 | 0.927 | 1 |
| RSAD2 IFI27 SLC1A2 IL1R2 OLAH MMP8 | 0.889 | 0.881 | 0.897 | 0.925 | 0.875 | 0.976 | 0.935 | 0.871 | 0.998 |
| RSAD2 IFI27 IFI44L SIGLEC1 OLAH MMP8 | 0.889 | 0.881 | 0.897 | 0.919 | 0.862 | 0.975 | 0.949 | 0.898 | 1.000 |
| IFI27 IFI44L ISG15 SLC1A2 IL1R2 FAM20A | 0.889 | 0.880 | 0.897 | 0.930 | 0.882 | 0.977 | 0.926 | 0.842 | 1 |
| RSAD2 IFI27 SLC1A2 IL1R2 FAM20A MMP8 | 0.889 | 0.881 | 0.897 | 0.929 | 0.882 | 0.976 | 0.926 | 0.839 | 1 |
| IFI27 IFI44L SIGLEC1 HERC6 SLC1A2 OLAH | 0.889 | 0.880 | 0.898 | 0.930 | 0.879 | 0.980 | 0.945 | 0.890 | 0.999 |
| IFI27 OAS1 ISG15 IL1R2 OLAH MMP8 | 0.889 | 0.881 | 0.897 | 0.923 | 0.871 | 0.974 | 0.939 | 0.883 | 0.995 |
| IFI27 IFIT1 SLC1A2 IL1R2 OLAH MMP8 | 0.889 | 0.880 | 0.898 | 0.926 | 0.876 | 0.976 | 0.935 | 0.872 | 0.998 |
| RSAD2 IFI27 IFIT1 SIGLEC1 SLC1A2 OLAH | 0.889 | 0.880 | 0.897 | 0.927 | 0.875 | 0.978 | 0.941 | 0.883 | 1.000 |
| RSAD2 IFI27 OAS1 SIGLEC1 OLAH MMP8 | 0.889 | 0.880 | 0.897 | 0.918 | 0.862 | 0.974 | 0.942 | 0.887 | 0.998 |
| RSAD2 IFI27 IFIT1 HERC6 SLC1A2 OLAH | 0.889 | 0.880 | 0.898 | 0.928 | 0.877 | 0.978 | 0.930 | 0.865 | 0.995 |
| RSAD2 IFI27 IFI44L HERC6 IL1R2 RETN | 0.889 | 0.881 | 0.897 | 0.921 | 0.868 | 0.974 | 0.936 | 0.876 | 0.996 |
| RSAD2 IFI27 IFIT1 OLAH RETN MMP8 | 0.889 | 0.880 | 0.898 | 0.919 | 0.859 | 0.978 | 0.952 | 0.896 | 1 |
| IFI27 IFI44L HERC6 SLC1A2 IL1R2 FAM20A | 0.889 | 0.880 | 0.898 | 0.929 | 0.881 | 0.977 | 0.928 | 0.843 | 1 |
| IFI27 OAS1 ISG15 OLAH RETN MMP8 | 0.889 | 0.880 | 0.898 | 0.918 | 0.859 | 0.978 | 0.949 | 0.893 | 1 |
| RSAD2 IFI27 IFIT1 SIGLEC1 OLAH MMP8 | 0.889 | 0.880 | 0.897 | 0.919 | 0.864 | 0.975 | 0.942 | 0.887 | 0.997 |
| IFI27 IFIT1 ISG15 SLC1A2 IL1R2 OLAH | 0.889 | 0.880 | 0.897 | 0.928 | 0.878 | 0.978 | 0.926 | 0.858 | 0.994 |
| IFI27 ISG15 HERC6 FAM20A RETN MMP8 | 0.889 | 0.879 | 0.899 | 0.920 | 0.861 | 0.980 | 0.941 | 0.864 | 1 |
| IFI27 IFIT1 IFI44L OLAH RETN MMP8 | 0.889 | 0.880 | 0.898 | 0.920 | 0.862 | 0.979 | 0.951 | 0.893 | 1 |
| RSAD2 IFI27 IFI44L SIGLEC1 ISG15 OLAH | 0.889 | 0.881 | 0.897 | 0.919 | 0.862 | 0.976 | 0.942 | 0.887 | 0.997 |
| RSAD2 IFI27 ISG15 HERC6 SLC1A2 OLAH | 0.889 | 0.880 | 0.897 | 0.928 | 0.878 | 0.978 | 0.924 | 0.855 | 0.993 |
| IFI27 OAS1 IFIT1 SLC1A2 OLAH MMP8 | 0.889 | 0.880 | 0.898 | 0.928 | 0.876 | 0.979 | 0.940 | 0.879 | 1 |
| IFI44L SIGLEC1 SLC1A2 IL1R2 FAM20A MMP8 | 0.889 | 0.880 | 0.897 | 0.924 | 0.876 | 0.973 | 0.957 | 0.889 | 1 |
| IFI27 OAS1 IFI44L SIGLEC1 IL1R2 RETN | 0.889 | 0.881 | 0.897 | 0.920 | 0.867 | 0.973 | 0.950 | 0.899 | 1 |
| OAS1 IFIT1 SIGLEC1 HERC6 OLAH FAM20A | 0.889 | 0.879 | 0.898 | 0.913 | 0.852 | 0.974 | 0.948 | 0.892 | 1 |
| IFIT1 SIGLEC1 ISG15 HERC6 OLAH FAM20A | 0.889 | 0.879 | 0.898 | 0.912 | 0.852 | 0.973 | 0.951 | 0.896 | 1 |
| IFI27 IFI44L ISG15 SLC1A2 IL1R2 OLAH | 0.889 | 0.880 | 0.897 | 0.927 | 0.878 | 0.977 | 0.925 | 0.854 | 0.996 |
| IFI27 IFIT1 HERC6 IL1R2 RETN MMP8 | 0.889 | 0.880 | 0.897 | 0.922 | 0.868 | 0.976 | 0.945 | 0.890 | 0.999 |
| IFI44L SIGLEC1 IL1R2 FAM20A RETN MMP8 | 0.889 | 0.880 | 0.898 | 0.918 | 0.860 | 0.976 | 0.962 | 0.905 | 1 |
| RSAD2 IFI27 OAS1 IFIT1 IL1R2 OLAH | 0.889 | 0.880 | 0.897 | 0.921 | 0.868 | 0.973 | 0.941 | 0.885 | 0.997 |
| RSAD2 SIGLEC1 ISG15 IL1R2 FAM20A MMP8 | 0.889 | 0.880 | 0.897 | 0.917 | 0.860 | 0.975 | 0.962 | 0.905 | 1 |
| IFI27 OAS1 IFIT1 SIGLEC1 IL1R2 RETN | 0.889 | 0.881 | 0.897 | 0.921 | 0.868 | 0.974 | 0.953 | 0.902 | 1 |
| RSAD2 IFI27 SIGLEC1 ISG15 OLAH MMP8 | 0.889 | 0.880 | 0.897 | 0.918 | 0.862 | 0.974 | 0.941 | 0.884 | 0.998 |
| IFIT1 SIGLEC1 ISG15 HERC6 FAM20A RETN | 0.889 | 0.880 | 0.898 | 0.916 | 0.855 | 0.976 | 0.942 | 0.875 | 1 |
| IFI27 IFIT1 IFI44L SIGLEC1 SLC1A2 OLAH | 0.889 | 0.880 | 0.897 | 0.926 | 0.875 | 0.978 | 0.946 | 0.890 | 1 |
| IFI27 OAS1 IFIT1 IL1R2 OLAH RETN | 0.889 | 0.880 | 0.897 | 0.918 | 0.861 | 0.974 | 0.945 | 0.886 | 1 |
| IFI27 IFIT1 SIGLEC1 HERC6 IL1R2 MMP8 | 0.889 | 0.881 | 0.896 | 0.921 | 0.874 | 0.969 | 0.948 | 0.900 | 0.996 |
| IFI27 SIGLEC1 HERC6 IL1R2 OLAH RETN | 0.889 | 0.880 | 0.897 | 0.918 | 0.861 | 0.976 | 0.948 | 0.893 | 1 |
| IFIT1 SIGLEC1 SLC1A2 IL1R2 FAM20A MMP8 | 0.889 | 0.880 | 0.897 | 0.927 | 0.880 | 0.974 | 0.960 | 0.898 | 1 |
| IFI27 SLC1A2 IL1R2 OLAH RETN MMP8 | 0.889 | 0.879 | 0.898 | 0.926 | 0.875 | 0.978 | 0.938 | 0.871 | 1 |
| IFI27 IFIT1 IFI44L HERC6 IL1R2 RETN | 0.889 | 0.880 | 0.897 | 0.920 | 0.866 | 0.973 | 0.935 | 0.874 | 0.995 |
| IFI27 IFIT1 SIGLEC1 HERC6 SLC1A2 FAM20A | 0.889 | 0.880 | 0.897 | 0.926 | 0.874 | 0.978 | 0.941 | 0.864 | 1 |
| RSAD2 IFI27 SIGLEC1 IL1R2 OLAH RETN | 0.889 | 0.880 | 0.897 | 0.919 | 0.862 | 0.975 | 0.948 | 0.892 | 1 |
| IFI27 OAS1 IFIT1 SIGLEC1 FAM20A MMP8 | 0.888 | 0.879 | 0.898 | 0.914 | 0.849 | 0.978 | 0.948 | 0.881 | 1 |
| IFI27 ISG15 HERC6 SLC1A2 IL1R2 OLAH | 0.888 | 0.880 | 0.897 | 0.928 | 0.879 | 0.977 | 0.926 | 0.858 | 0.994 |
| IFI27 SIGLEC1 SLC1A2 OLAH RETN MMP8 | 0.888 | 0.879 | 0.898 | 0.924 | 0.869 | 0.979 | 0.957 | 0.906 | 1 |
| IFI27 HERC6 SLC1A2 IL1R2 FAM20A MMP8 | 0.888 | 0.880 | 0.897 | 0.931 | 0.885 | 0.977 | 0.925 | 0.839 | 1 |
| IFI27 IFI44L HERC6 FAM20A RETN MMP8 | 0.888 | 0.878 | 0.898 | 0.920 | 0.860 | 0.980 | 0.940 | 0.862 | 1 |
| RSAD2 SIGLEC1 SLC1A2 IL1R2 FAM20A MMP8 | 0.888 | 0.879 | 0.897 | 0.924 | 0.875 | 0.972 | 0.959 | 0.895 | 1 |
| IFI27 IFIT1 IFI44L SIGLEC1 ISG15 IL1R2 | 0.888 | 0.881 | 0.896 | 0.920 | 0.871 | 0.969 | 0.945 | 0.894 | 0.995 |
| IFIT1 IFI44L SIGLEC1 HERC6 FAM20A RETN | 0.888 | 0.880 | 0.897 | 0.914 | 0.853 | 0.976 | 0.936 | 0.869 | 1 |
| IFI27 OAS1 IFI44L SIGLEC1 SLC1A2 OLAH | 0.888 | 0.880 | 0.897 | 0.927 | 0.876 | 0.978 | 0.948 | 0.893 | 1 |
| IFI27 IFI44L SLC1A2 OLAH RETN MMP8 | 0.888 | 0.879 | 0.897 | 0.925 | 0.871 | 0.980 | 0.943 | 0.881 | 1 |
| SIGLEC1 SLC1A2 IL1R2 OLAH FAM20A MMP8 | 0.888 | 0.879 | 0.897 | 0.922 | 0.872 | 0.972 | 0.957 | 0.890 | 1 |
| OAS1 SIGLEC1 ISG15 IL1R2 FAM20A MMP8 | 0.888 | 0.879 | 0.897 | 0.915 | 0.857 | 0.973 | 0.963 | 0.906 | 1 |
| RSAD2 IFI27 ISG15 SLC1A2 OLAH MMP8 | 0.888 | 0.880 | 0.897 | 0.927 | 0.875 | 0.979 | 0.928 | 0.860 | 0.996 |
| RSAD2 IFI27 OAS1 SLC1A2 OLAH MMP8 | 0.888 | 0.879 | 0.897 | 0.926 | 0.874 | 0.978 | 0.935 | 0.869 | 1 |
| IFI27 OAS1 ISG15 SLC1A2 OLAH MMP8 | 0.888 | 0.880 | 0.897 | 0.927 | 0.875 | 0.979 | 0.922 | 0.850 | 0.993 |
| IFI27 HERC6 SLC1A2 OLAH RETN MMP8 | 0.888 | 0.879 | 0.897 | 0.924 | 0.870 | 0.979 | 0.946 | 0.887 | 1 |
| RSAD2 IFI27 ISG15 IL1R2 OLAH MMP8 | 0.888 | 0.880 | 0.896 | 0.922 | 0.869 | 0.974 | 0.943 | 0.890 | 0.997 |
| RSAD2 IFI27 IFIT1 SLC1A2 OLAH MMP8 | 0.888 | 0.880 | 0.897 | 0.927 | 0.875 | 0.978 | 0.936 | 0.873 | 0.999 |
| RSAD2 IFI27 IFIT1 SIGLEC1 OLAH RETN | 0.888 | 0.880 | 0.897 | 0.916 | 0.857 | 0.974 | 0.950 | 0.893 | 1 |
| IFI27 SIGLEC1 HERC6 SLC1A2 IL1R2 OLAH | 0.888 | 0.879 | 0.897 | 0.927 | 0.877 | 0.978 | 0.940 | 0.883 | 0.998 |
| IFI27 OAS1 IFIT1 ISG15 SLC1A2 OLAH | 0.888 | 0.879 | 0.897 | 0.927 | 0.875 | 0.978 | 0.932 | 0.865 | 0.998 |
| RSAD2 IFI27 OAS1 IFI44L SIGLEC1 OLAH | 0.888 | 0.880 | 0.896 | 0.918 | 0.861 | 0.975 | 0.950 | 0.900 | 1 |
| RSAD2 IFI27 IFI44L SIGLEC1 OLAH RETN | 0.888 | 0.880 | 0.897 | 0.916 | 0.856 | 0.976 | 0.951 | 0.896 | 1 |
| RSAD2 IFI27 OAS1 SIGLEC1 IL1R2 RETN | 0.888 | 0.880 | 0.896 | 0.919 | 0.865 | 0.973 | 0.955 | 0.901 | 1 |
| IFI27 IFIT1 HERC6 SLC1A2 OLAH MMP8 | 0.888 | 0.879 | 0.897 | 0.928 | 0.878 | 0.979 | 0.935 | 0.873 | 0.997 |
| IFI27 IFIT1 HERC6 SLC1A2 IL1R2 FAM20A | 0.888 | 0.879 | 0.897 | 0.930 | 0.882 | 0.977 | 0.927 | 0.842 | 1 |
| IFI27 IFIT1 IFI44L SLC1A2 IL1R2 FAM20A | 0.888 | 0.879 | 0.897 | 0.924 | 0.874 | 0.975 | 0.927 | 0.843 | 1 |
| IFI27 OAS1 IFIT1 HERC6 IL1R2 MMP8 | 0.888 | 0.880 | 0.896 | 0.917 | 0.868 | 0.966 | 0.937 | 0.881 | 0.993 |
| IFI27 OAS1 IFI44L IL1R2 OLAH MMP8 | 0.888 | 0.880 | 0.896 | 0.922 | 0.870 | 0.974 | 0.941 | 0.886 | 0.996 |
| IFI27 IFIT1 IFI44L SIGLEC1 IL1R2 MMP8 | 0.888 | 0.880 | 0.896 | 0.922 | 0.873 | 0.970 | 0.946 | 0.897 | 0.995 |
| RSAD2 IFI27 OAS1 IFIT1 SIGLEC1 OLAH | 0.888 | 0.880 | 0.896 | 0.919 | 0.864 | 0.975 | 0.940 | 0.884 | 0.997 |
| IFI27 OAS1 SIGLEC1 ISG15 OLAH RETN | 0.888 | 0.879 | 0.897 | 0.917 | 0.859 | 0.975 | 0.948 | 0.890 | 1 |
| RSAD2 IFI27 ISG15 SLC1A2 IL1R2 OLAH | 0.888 | 0.880 | 0.896 | 0.927 | 0.877 | 0.978 | 0.928 | 0.860 | 0.996 |
| IFI27 OAS1 IFIT1 SLC1A2 IL1R2 OLAH | 0.888 | 0.879 | 0.897 | 0.927 | 0.876 | 0.977 | 0.933 | 0.866 | 1.000 |
| IFI27 IFI44L ISG15 IL1R2 OLAH RETN | 0.888 | 0.880 | 0.896 | 0.918 | 0.862 | 0.974 | 0.940 | 0.879 | 1 |
| IFI27 ISG15 HERC6 SLC1A2 OLAH RETN | 0.888 | 0.879 | 0.897 | 0.926 | 0.875 | 0.977 | 0.934 | 0.869 | 0.999 |
| RSAD2 IFI27 IFI44L IL1R2 OLAH MMP8 | 0.888 | 0.880 | 0.896 | 0.922 | 0.870 | 0.974 | 0.945 | 0.892 | 0.998 |
| IFI27 ISG15 SLC1A2 OLAH RETN MMP8 | 0.888 | 0.879 | 0.897 | 0.927 | 0.874 | 0.979 | 0.937 | 0.870 | 1 |
| RSAD2 IFI27 OAS1 SIGLEC1 OLAH RETN | 0.888 | 0.879 | 0.897 | 0.916 | 0.858 | 0.974 | 0.949 | 0.888 | 1 |
| IFI27 OAS1 SLC1A2 IL1R2 OLAH MMP8 | 0.888 | 0.880 | 0.896 | 0.926 | 0.876 | 0.976 | 0.930 | 0.864 | 0.997 |
| IFI27 HERC6 SLC1A2 IL1R2 OLAH MMP8 | 0.888 | 0.879 | 0.896 | 0.927 | 0.878 | 0.977 | 0.933 | 0.869 | 0.996 |
| IFI27 ISG15 SLC1A2 IL1R2 OLAH RETN | 0.888 | 0.879 | 0.896 | 0.926 | 0.876 | 0.977 | 0.928 | 0.857 | 1.000 |
| IFI27 OAS1 IFI44L OLAH RETN MMP8 | 0.888 | 0.879 | 0.897 | 0.918 | 0.859 | 0.978 | 0.950 | 0.894 | 1 |
| IFI27 OAS1 IFIT1 ISG15 OLAH MMP8 | 0.888 | 0.879 | 0.897 | 0.914 | 0.855 | 0.973 | 0.935 | 0.873 | 0.996 |
| RSAD2 IFI27 SIGLEC1 ISG15 HERC6 IL1R2 | 0.888 | 0.880 | 0.896 | 0.921 | 0.874 | 0.98 | 0.948 | 0.900 | 0.996 |
| RSAD2 SIGLEC1 IL1R2 FAM20A RETN MMP8 | 0.888 | 0.879 | 0.897 | 0.920 | 0.863 | 0.977 | 0.964 | 0.912 | 1 |
| IFI27 IFIT1 HERC6 SLC1A2 OLAH RETN | 0.888 | 0.878 | 0.897 | 0.925 | 0.874 | 0.977 | 0.936 | 0.871 | 1 |
| RSAD2 IFI27 OAS1 IL1R2 OLAH RETN | 0.888 | 0.879 | 0.896 | 0.918 | 0.862 | 0.974 | 0.945 | 0.887 | 1 |
| IFI27 IFIT1 SIGLEC1 HERC6 RETN MMP8 | 0.888 | 0.879 | 0.897 | 0.910 | 0.848 | 0.972 | 0.941 | 0.885 | 0.997 |
| RSAD2 IFI27 SIGLEC1 HERC6 RETN MMP8 | 0.888 | 0.878 | 0.897 | 0.915 | 0.856 | 0.975 | 0.952 | 0.902 | 1 |
| IFI27 OAS1 IFIT1 ISG15 IL1R2 OLAH | 0.888 | 0.879 | 0.896 | 0.919 | 0.865 | 0.972 | 0.941 | 0.885 | 0.998 |
| IFI27 OAS1 SIGLEC1 SLC1A2 FAM20A RETN | 0.888 | 0.878 | 0.897 | 0.922 | 0.864 | 0.979 | 0.939 | 0.862 | 1 |
| IFI27 ISG15 HERC6 SLC1A2 IL1R2 FAM20A | 0.888 | 0.878 | 0.897 | 0.930 | 0.883 | 0.977 | 0.923 | 0.835 | 1 |
| RSAD2 IFI27 IFI44L SIGLEC1 SLC1A2 OLAH | 0.888 | 0.879 | 0.896 | 0.926 | 0.875 | 0.978 | 0.946 | 0.890 | 1 |
| RSAD2 IFI27 IFIT1 IFI44L SIGLEC1 OLAH | 0.888 | 0.879 | 0.896 | 0.919 | 0.862 | 0.975 | 0.949 | 0.898 | 1.000 |
| IFI27 IFIT1 SIGLEC1 ISG15 IL1R2 RETN | 0.888 | 0.880 | 0.896 | 0.920 | 0.867 | 0.973 | 0.957 | 0.912 | 1 |
| IFI27 IFIT1 IFI44L ISG15 OLAH MMP8 | 0.888 | 0.879 | 0.896 | 0.915 | 0.857 | 0.974 | 0.936 | 0.876 | 0.995 |
| RSAD2 IFI27 ISG15 HERC6 IL1R2 RETN | 0.888 | 0.879 | 0.896 | 0.920 | 0.868 | 0.973 | 0.938 | 0.880 | 0.996 |
| IFI27 OAS1 IFIT1 ISG15 OLAH RETN | 0.888 | 0.879 | 0.896 | 0.916 | 0.857 | 0.975 | 0.940 | 0.878 | 1 |
| RSAD2 IFI27 OAS1 IFIT1 HERC6 IL1R2 | 0.888 | 0.880 | 0.896 | 0.917 | 0.868 | 0.966 | 0.941 | 0.888 | 0.995 |
| RSAD2 IFI27 OAS1 ISG15 IL1R2 OLAH | 0.888 | 0.879 | 0.896 | 0.920 | 0.867 | 0.973 | 0.943 | 0.890 | 0.997 |
| SIGLEC1 HERC6 IL1R2 FAM20A RETN MMP8 | 0.888 | 0.878 | 0.897 | 0.918 | 0.859 | 0.976 | 0.960 | 0.898 | 1 |
| IFI27 IFI44L HERC6 SLC1A2 OLAH MMP8 | 0.888 | 0.878 | 0.897 | 0.927 | 0.877 | 0.978 | 0.932 | 0.868 | 0.995 |
| OAS1 IFIT1 SIGLEC1 IL1R2 OLAH FAM20A | 0.888 | 0.879 | 0.896 | 0.914 | 0.852 | 0.977 | 0.963 | 0.914 | 1 |
| IFI27 IFI44L SLC1A2 IL1R2 OLAH RETN | 0.888 | 0.879 | 0.896 | 0.926 | 0.875 | 0.977 | 0.935 | 0.868 | 1 |
| IFI27 IFIT1 ISG15 IL1R2 OLAH RETN | 0.888 | 0.879 | 0.896 | 0.918 | 0.862 | 0.975 | 0.941 | 0.882 | 1 |
| RSAD2 IFI27 SIGLEC1 SLC1A2 OLAH RETN | 0.888 | 0.879 | 0.896 | 0.927 | 0.875 | 0.979 | 0.949 | 0.891 | 1 |
| IFIT1 SIGLEC1 SLC1A2 OLAH FAM20A MMP8 | 0.888 | 0.878 | 0.897 | 0.923 | 0.870 | 0.975 | 0.952 | 0.895 | 1 |
| RSAD2 IFI27 OAS1 SLC1A2 IL1R2 OLAH | 0.888 | 0.879 | 0.896 | 0.926 | 0.876 | 0.976 | 0.930 | 0.862 | 0.999 |
| SIGLEC1 ISG15 IL1R2 FAM20A RETN MMP8 | 0.888 | 0.879 | 0.896 | 0.915 | 0.855 | 0.975 | 0.962 | 0.902 | 1 |
| IFI27 OAS1 IFIT1 IFI44L SIGLEC1 IL1R2 | 0.888 | 0.880 | 0.895 | 0.921 | 0.873 | 0.970 | 0.948 | 0.900 | 0.996 |
| SIGLEC1 IL1R2 OLAH FAM20A RETN MMP8 | 0.887 | 0.878 | 0.897 | 0.918 | 0.860 | 0.977 | 0.963 | 0.909 | 1 |
| RSAD2 IFI27 OAS1 SIGLEC1 ISG15 OLAH | 0.887 | 0.879 | 0.896 | 0.918 | 0.862 | 0.974 | 0.941 | 0.884 | 0.999 |
| SIGLEC1 ISG15 SLC1A2 IL1R2 FAM20A MMP8 | 0.887 | 0.879 | 0.896 | 0.924 | 0.877 | 0.972 | 0.958 | 0.890 | 1 |
| IFI27 IFIT1 SLC1A2 OLAH RETN MMP8 | 0.887 | 0.878 | 0.897 | 0.924 | 0.869 | 0.979 | 0.947 | 0.887 | 1 |
| IFIT1 SIGLEC1 HERC6 OLAH FAM20A RETN | 0.887 | 0.878 | 0.897 | 0.915 | 0.854 | 0.976 | 0.946 | 0.886 | 1 |
| IFI27 SIGLEC1 ISG15 HERC6 IL1R2 RETN | 0.887 | 0.879 | 0.896 | 0.923 | 0.870 | 0.975 | 0.952 | 0.905 | 1.000 |
| IFI27 OAS1 IFI44L SIGLEC1 IL1R2 MMP8 | 0.887 | 0.880 | 0.895 | 0.919 | 0.870 | 0.968 | 0.943 | 0.894 | 0.993 |
| SIGLEC1 HERC6 SLC1A2 IL1R2 FAM20A MMP8 | 0.887 | 0.878 | 0.897 | 0.923 | 0.873 | 0.973 | 0.959 | 0.893 | 1 |
| OAS1 SIGLEC1 IL1R2 FAM20A RETN MMP8 | 0.887 | 0.878 | 0.896 | 0.918 | 0.859 | 0.976 | 0.964 | 0.912 | 1 |
| RSAD2 IFI27 IFIT1 ISG15 SLC1A2 OLAH | 0.887 | 0.879 | 0.896 | 0.926 | 0.874 | 0.978 | 0.928 | 0.861 | 0.996 |
| RSAD2 IFI27 IFIT1 SLC1A2 IL1R2 OLAH | 0.887 | 0.879 | 0.896 | 0.927 | 0.877 | 0.977 | 0.933 | 0.868 | 0.998 |
| RSAD2 IFI27 OAS1 IFIT1 OLAH MMP8 | 0.887 | 0.879 | 0.896 | 0.915 | 0.857 | 0.973 | 0.938 | 0.879 | 0.997 |
| RSAD2 IFI27 OAS1 ISG15 OLAH MMP8 | 0.887 | 0.879 | 0.896 | 0.916 | 0.858 | 0.974 | 0.936 | 0.877 | 0.995 |
| RSAD2 IFIT1 SIGLEC1 HERC6 OLAH FAM20A | 0.887 | 0.878 | 0.897 | 0.912 | 0.851 | 0.973 | 0.951 | 0.896 | 1 |
| IFI27 IFI44L ISG15 HERC6 SLC1A2 OLAH | 0.887 | 0.878 | 0.896 | 0.928 | 0.878 | 0.978 | 0.924 | 0.855 | 0.993 |
| OAS1 SIGLEC1 SLC1A2 IL1R2 FAM20A MMP8 | 0.887 | 0.878 | 0.896 | 0.923 | 0.874 | 0.971 | 0.961 | 0.899 | 1 |
| IFI27 IFIT1 IFI44L ISG15 SLC1A2 OLAH | 0.887 | 0.879 | 0.896 | 0.926 | 0.874 | 0.978 | 0.927 | 0.858 | 0.996 |
| IFI27 IFIT1 IFI44L ISG15 IL1R2 OLAH | 0.887 | 0.879 | 0.895 | 0.918 | 0.864 | 0.973 | 0.938 | 0.881 | 0.995 |
| IFI27 IFIT1 ISG15 SLC1A2 OLAH RETN | 0.887 | 0.879 | 0.896 | 0.928 | 0.877 | 0.979 | 0.934 | 0.866 | 1 |
| RSAD2 IFI27 OAS1 HERC6 RETN MMP8 | 0.887 | 0.878 | 0.897 | 0.915 | 0.853 | 0.977 | 0.934 | 0.862 | 1 |
| IFI27 IFIT1 SIGLEC1 ISG15 HERC6 IL1R2 | 0.887 | 0.880 | 0.895 | 0.922 | 0.875 | 0.969 | 0.950 | 0.903 | 0.997 |
| IFI27 OAS1 IFIT1 SIGLEC1 IL1R2 MMP8 | 0.887 | 0.879 | 0.895 | 0.919 | 0.870 | 0.967 | 0.952 | 0.906 | 0.998 |
| IFI27 SIGLEC1 ISG15 HERC6 IL1R2 MMP8 | 0.887 | 0.880 | 0.895 | 0.920 | 0.872 | 0.968 | 0.949 | 0.902 | 0.996 |
| IFI27 SIGLEC1 SLC1A2 IL1R2 OLAH RETN | 0.887 | 0.878 | 0.896 | 0.926 | 0.874 | 0.977 | 0.949 | 0.894 | 1 |
| IFI27 OAS1 IFIT1 IFI44L IL1R2 OLAH | 0.887 | 0.879 | 0.895 | 0.921 | 0.868 | 0.975 | 0.940 | 0.885 | 0.995 |
| IFI27 ISG15 HERC6 IL1R2 RETN MMP8 | 0.887 | 0.879 | 0.896 | 0.921 | 0.867 | 0.975 | 0.946 | 0.892 | 0.999 |
| IFI27 OAS1 SLC1A2 IL1R2 FAM20A MMP8 | 0.887 | 0.879 | 0.896 | 0.927 | 0.878 | 0.976 | 0.927 | 0.841 | 1 |
| RSAD2 IFI27 IFI44L SIGLEC1 IL1R2 RETN | 0.887 | 0.879 | 0.895 | 0.919 | 0.864 | 0.974 | 0.950 | 0.900 | 1 |
| RSAD2 IFI27 ISG15 OLAH RETN MMP8 | 0.887 | 0.878 | 0.896 | 0.918 | 0.858 | 0.978 | 0.949 | 0.892 | 1 |
| RSAD2 IFI27 IFIT1 SIGLEC1 ISG15 OLAH | 0.887 | 0.879 | 0.895 | 0.918 | 0.862 | 0.974 | 0.941 | 0.885 | 0.998 |
| IFI27 IFI44L SIGLEC1 SLC1A2 IL1R2 RETN | 0.887 | 0.879 | 0.895 | 0.923 | 0.874 | 0.973 | 0.949 | 0.896 | 1 |
| IFI27 OAS1 IFIT1 IFI44L OLAH RETN | 0.887 | 0.878 | 0.896 | 0.914 | 0.855 | 0.974 | 0.945 | 0.884 | 1 |
| IFI27 IFI44L HERC6 SLC1A2 OLAH RETN | 0.887 | 0.878 | 0.896 | 0.924 | 0.871 | 0.977 | 0.936 | 0.871 | 1 |
| IFI27 OAS1 IFI44L HERC6 SLC1A2 OLAH | 0.887 | 0.878 | 0.897 | 0.929 | 0.880 | 0.978 | 0.929 | 0.861 | 0.997 |
| IFI27 IFI44L SIGLEC1 ISG15 HERC6 RETN | 0.887 | 0.878 | 0.896 | 0.911 | 0.849 | 0.973 | 0.942 | 0.885 | 1.000 |
| RSAD2 IFI27 OAS1 IFIT1 SLC1A2 OLAH | 0.887 | 0.878 | 0.896 | 0.926 | 0.875 | 0.978 | 0.940 | 0.877 | 1 |
| IFI27 SLC1A2 IL1R2 FAM20A RETN MMP8 | 0.887 | 0.878 | 0.896 | 0.925 | 0.877 | 0.974 | 0.909 | 0.814 | 1 |
| IFI27 ISG15 SLC1A2 IL1R2 FAM20A MMP8 | 0.887 | 0.879 | 0.895 | 0.926 | 0.877 | 0.975 | 0.916 | 0.825 | 1 |
| RSAD2 IFI27 IFI44L SLC1A2 IL1R2 FAM20A | 0.887 | 0.878 | 0.896 | 0.927 | 0.878 | 0.976 | 0.928 | 0.844 | 1 |
| IFI27 IFI44L ISG15 SLC1A2 OLAH RETN | 0.887 | 0.878 | 0.896 | 0.929 | 0.878 | 0.979 | 0.933 | 0.865 | 1 |
| RSAD2 IFI27 OAS1 HERC6 IL1R2 MMP8 | 0.887 | 0.879 | 0.895 | 0.919 | 0.871 | 0.967 | 0.933 | 0.875 | 0.990 |
| IFI27 OAS1 SIGLEC1 HERC6 SLC1A2 IL1R2 | 0.887 | 0.879 | 0.895 | 0.927 | 0.882 | 0.971 | 0.949 | 0.899 | 0.999 |
| IFI27 SIGLEC1 HERC6 SLC1A2 OLAH RETN | 0.887 | 0.877 | 0.896 | 0.926 | 0.873 | 0.978 | 0.946 | 0.888 | 1 |
| RSAD2 IFI27 IFI44L SLC1A2 OLAH MMP8 | 0.887 | 0.878 | 0.896 | 0.927 | 0.875 | 0.978 | 0.932 | 0.866 | 0.997 |
| IFI27 OAS1 SLC1A2 OLAH RETN MMP8 | 0.887 | 0.878 | 0.896 | 0.924 | 0.870 | 0.978 | 0.941 | 0.878 | 1 |
| IFI27 IFI44L SIGLEC1 ISG15 HERC6 FAM20A | 0.887 | 0.878 | 0.896 | 0.913 | 0.850 | 0.976 | 0.943 | 0.866 | 1 |
| IFI27 OAS1 SIGLEC1 ISG15 IL1R2 RETN | 0.887 | 0.879 | 0.895 | 0.921 | 0.868 | 0.974 | 0.954 | 0.904 | 1 |
| RSAD2 IFI27 SLC1A2 OLAH RETN MMP8 | 0.887 | 0.878 | 0.896 | 0.925 | 0.871 | 0.979 | 0.942 | 0.879 | 1 |
| IFI27 IFIT1 IFI44L SLC1A2 OLAH MMP8 | 0.887 | 0.878 | 0.896 | 0.926 | 0.874 | 0.978 | 0.936 | 0.873 | 0.999 |
| RSAD2 IFI27 OAS1 SIGLEC1 ISG15 FAM20A | 0.887 | 0.877 | 0.897 | 0.915 | 0.851 | 0.979 | 0.947 | 0.876 | 1 |
| IFI27 OAS1 IFI44L ISG15 OLAH MMP8 | 0.887 | 0.878 | 0.895 | 0.917 | 0.859 | 0.975 | 0.934 | 0.874 | 0.994 |
| OAS1 IFIT1 SIGLEC1 HERC6 FAM20A RETN | 0.887 | 0.878 | 0.896 | 0.914 | 0.853 | 0.975 | 0.940 | 0.873 | 1 |
| IFIT1 IFI44L SIGLEC1 OLAH FAM20A MMP8 | 0.887 | 0.877 | 0.896 | 0.908 | 0.843 | 0.974 | 0.954 | 0.904 | 1 |
| RSAD2 IFI27 IFI44L SIGLEC1 IL1R2 MMP8 | 0.887 | 0.879 | 0.895 | 0.919 | 0.869 | 0.970 | 0.943 | 0.893 | 0.994 |
| IFI27 OAS1 IFI44L SLC1A2 IL1R2 FAM20A | 0.887 | 0.878 | 0.896 | 0.929 | 0.881 | 0.976 | 0.927 | 0.843 | 1 |
| IFI27 IFIT1 HERC6 SLC1A2 IL1R2 OLAH | 0.887 | 0.878 | 0.896 | 0.926 | 0.876 | 0.976 | 0.934 | 0.870 | 0.997 |
| IFI27 OAS1 IFIT1 IFI44L HERC6 IL1R2 | 0.887 | 0.879 | 0.895 | 0.922 | 0.873 | 0.970 | 0.938 | 0.883 | 0.993 |
| RSAD2 IFI27 SLC1A2 IL1R2 OLAH RETN | 0.887 | 0.878 | 0.896 | 0.923 | 0.871 | 0.975 | 0.934 | 0.866 | 1 |
| RSAD2 IFI27 OAS1 IFI44L SIGLEC1 FAM20A | 0.887 | 0.877 | 0.896 | 0.914 | 0.850 | 0.979 | 0.949 | 0.879 | 1 |
| IFI27 OAS1 IFIT1 IFI44L OLAH MMP8 | 0.887 | 0.878 | 0.895 | 0.916 | 0.858 | 0.974 | 0.941 | 0.884 | 0.999 |
| RSAD2 IFI27 IFI44L ISG15 SLC1A2 OLAH | 0.887 | 0.878 | 0.895 | 0.927 | 0.875 | 0.979 | 0.928 | 0.860 | 0.996 |
| IFI27 OAS1 SIGLEC1 SLC1A2 IL1R2 RETN | 0.887 | 0.878 | 0.895 | 0.924 | 0.876 | 0.973 | 0.952 | 0.897 | 1 |
| IFI27 IFIT1 IFI44L IL1R2 OLAH RETN | 0.887 | 0.878 | 0.895 | 0.916 | 0.860 | 0.973 | 0.942 | 0.883 | 1 |
| IFI27 IFIT1 IFI44L SIGLEC1 SLC1A2 FAM20A | 0.887 | 0.878 | 0.896 | 0.921 | 0.863 | 0.978 | 0.941 | 0.865 | 1 |
| RSAD2 IFI27 IFI44L ISG15 IL1R2 OLAH | 0.887 | 0.879 | 0.895 | 0.922 | 0.869 | 0.975 | 0.941 | 0.885 | 0.997 |
| IFI27 IFIT1 SLC1A2 IL1R2 OLAH RETN | 0.887 | 0.878 | 0.895 | 0.923 | 0.870 | 0.975 | 0.934 | 0.866 | 1 |
| OAS1 IFIT1 IFI44L SIGLEC1 IL1R2 FAM20A | 0.887 | 0.878 | 0.896 | 0.913 | 0.850 | 0.976 | 0.959 | 0.907 | 1 |
| IFI27 OAS1 ISG15 IL1R2 OLAH RETN | 0.887 | 0.878 | 0.895 | 0.919 | 0.863 | 0.974 | 0.939 | 0.879 | 0.999 |
| IFI27 HERC6 SLC1A2 IL1R2 OLAH RETN | 0.887 | 0.878 | 0.896 | 0.924 | 0.871 | 0.976 | 0.938 | 0.874 | 1 |
| IFI27 OAS1 IFIT1 SLC1A2 IL1R2 FAM20A | 0.887 | 0.878 | 0.896 | 0.925 | 0.875 | 0.975 | 0.925 | 0.839 | 1 |
| RSAD2 IFI27 OAS1 IFIT1 ISG15 OLAH | 0.887 | 0.878 | 0.895 | 0.915 | 0.856 | 0.973 | 0.934 | 0.872 | 0.995 |
| RSAD2 IFI27 IFIT1 SLC1A2 IL1R2 FAM20A | 0.887 | 0.878 | 0.895 | 0.924 | 0.873 | 0.975 | 0.927 | 0.842 | 1 |
| RSAD2 IFI27 SIGLEC1 ISG15 OLAH RETN | 0.887 | 0.878 | 0.895 | 0.917 | 0.860 | 0.975 | 0.947 | 0.889 | 1 |
| IFI27 OAS1 IFI44L ISG15 IL1R2 OLAH | 0.887 | 0.878 | 0.895 | 0.923 | 0.871 | 0.975 | 0.936 | 0.878 | 0.994 |
| RSAD2 IFI27 IFIT1 ISG15 IL1R2 OLAH | 0.887 | 0.879 | 0.895 | 0.919 | 0.865 | 0.972 | 0.941 | 0.885 | 0.998 |
| IFI27 IFIT1 ISG15 HERC6 IL1R2 RETN | 0.887 | 0.878 | 0.895 | 0.919 | 0.865 | 0.973 | 0.940 | 0.884 | 0.997 |
| RSAD2 IFI27 IFIT1 SIGLEC1 FAM20A MMP8 | 0.887 | 0.877 | 0.896 | 0.913 | 0.848 | 0.978 | 0.946 | 0.879 | 1 |
| RSAD2 IFI27 OAS1 IFI44L SIGLEC1 IL1R2 | 0.887 | 0.879 | 0.894 | 0.921 | 0.871 | 0.971 | 0.951 | 0.905 | 0.997 |
| IFI27 IFIT1 IFI44L ISG15 OLAH RETN | 0.887 | 0.878 | 0.895 | 0.915 | 0.856 | 0.975 | 0.941 | 0.879 | 1 |
| IFI27 OAS1 IFIT1 IL1R2 RETN MMP8 | 0.887 | 0.878 | 0.895 | 0.918 | 0.863 | 0.974 | 0.954 | 0.904 | 1 |
| IFI27 OAS1 IFI44L HERC6 IL1R2 MMP8 | 0.887 | 0.879 | 0.894 | 0.916 | 0.867 | 0.965 | 0.930 | 0.872 | 0.989 |
| IFI27 IFIT1 IFI44L IL1R2 RETN MMP8 | 0.886 | 0.878 | 0.895 | 0.918 | 0.863 | 0.974 | 0.946 | 0.891 | 1 |
| RSAD2 IFI27 IFIT1 SIGLEC1 IL1R2 RETN | 0.886 | 0.878 | 0.895 | 0.920 | 0.866 | 0.973 | 0.959 | 0.915 | 1 |
| IFI27 OAS1 IFI44L HERC6 RETN MMP8 | 0.886 | 0.877 | 0.896 | 0.914 | 0.853 | 0.976 | 0.937 | 0.870 | 1 |
| RSAD2 IFI27 IFI44L SIGLEC1 ISG15 FAM20A | 0.886 | 0.877 | 0.896 | 0.914 | 0.849 | 0.978 | 0.945 | 0.872 | 1 |
| RSAD2 IFI27 IFI44L OLAH RETN MMP8 | 0.886 | 0.877 | 0.895 | 0.918 | 0.859 | 0.978 | 0.951 | 0.894 | 1 |
| RSAD2 IFI27 OAS1 IFI44L IL1R2 OLAH | 0.886 | 0.878 | 0.895 | 0.921 | 0.868 | 0.974 | 0.941 | 0.886 | 0.997 |
| IFI27 OAS1 IFIT1 HERC6 RETN MMP8 | 0.886 | 0.877 | 0.896 | 0.916 | 0.855 | 0.976 | 0.932 | 0.860 | 1 |
| IFI27 OAS1 ISG15 SLC1A2 IL1R2 OLAH | 0.886 | 0.878 | 0.895 | 0.926 | 0.875 | 0.977 | 0.920 | 0.848 | 0.991 |
| RSAD2 IFI27 IFIT1 IL1R2 OLAH RETN | 0.886 | 0.878 | 0.895 | 0.918 | 0.861 | 0.974 | 0.943 | 0.885 | 1 |
| RSAD2 IFI27 OAS1 ISG15 SLC1A2 OLAH | 0.886 | 0.878 | 0.895 | 0.926 | 0.874 | 0.979 | 0.930 | 0.862 | 0.999 |
| RSAD2 IFI27 OAS1 IFIT1 OLAH RETN | 0.886 | 0.878 | 0.895 | 0.914 | 0.853 | 0.974 | 0.947 | 0.887 | 1 |
| IFIT1 IFI44L SIGLEC1 ISG15 HERC6 FAM20A | 0.886 | 0.878 | 0.895 | 0.914 | 0.853 | 0.975 | 0.937 | 0.865 | 1 |
| IFI27 OAS1 SLC1A2 IL1R2 OLAH RETN | 0.886 | 0.878 | 0.895 | 0.926 | 0.874 | 0.977 | 0.933 | 0.865 | 1 |
| RSAD2 IFI27 OAS1 IFI44L HERC6 IL1R2 | 0.886 | 0.878 | 0.894 | 0.919 | 0.871 | 0.968 | 0.934 | 0.876 | 0.992 |
| IFI27 OAS1 HERC6 SLC1A2 IL1R2 RETN | 0.886 | 0.878 | 0.895 | 0.924 | 0.876 | 0.973 | 0.928 | 0.858 | 0.999 |
| IFI27 OAS1 IFI44L IL1R2 OLAH RETN | 0.886 | 0.878 | 0.895 | 0.918 | 0.862 | 0.974 | 0.941 | 0.883 | 1.000 |
| RSAD2 IFI27 IFI44L HERC6 SLC1A2 OLAH | 0.886 | 0.877 | 0.895 | 0.927 | 0.877 | 0.978 | 0.928 | 0.862 | 0.994 |
| RSAD2 IFI27 ISG15 IL1R2 OLAH RETN | 0.886 | 0.878 | 0.895 | 0.919 | 0.862 | 0.975 | 0.940 | 0.881 | 1.000 |
| RSAD2 IFI27 IFIT1 IFI44L IL1R2 OLAH | 0.886 | 0.878 | 0.894 | 0.921 | 0.867 | 0.974 | 0.940 | 0.885 | 0.995 |
| IFI27 OAS1 IFIT1 SLC1A2 OLAH RETN | 0.886 | 0.877 | 0.895 | 0.926 | 0.874 | 0.978 | 0.939 | 0.871 | 1 |
| IFI27 IFI44L HERC6 SLC1A2 IL1R2 OLAH | 0.886 | 0.877 | 0.896 | 0.926 | 0.877 | 0.976 | 0.930 | 0.866 | 0.995 |
| IFI44L SIGLEC1 ISG15 HERC6 IL1R2 FAM20A | 0.886 | 0.877 | 0.895 | 0.914 | 0.853 | 0.975 | 0.954 | 0.892 | 1 |
| IFI27 OAS1 IFI44L SLC1A2 OLAH MMP8 | 0.886 | 0.877 | 0.895 | 0.927 | 0.875 | 0.978 | 0.935 | 0.871 | 0.999 |
| IFI27 OAS1 IFI44L SIGLEC1 ISG15 FAM20A | 0.886 | 0.877 | 0.895 | 0.912 | 0.848 | 0.976 | 0.945 | 0.873 | 1 |
| IFIT1 SIGLEC1 HERC6 SLC1A2 IL1R2 FAM20A | 0.886 | 0.877 | 0.895 | 0.924 | 0.875 | 0.973 | 0.946 | 0.874 | 1 |
| RSAD2 IFI27 IFIT1 HERC6 IL1R2 MMP8 | 0.886 | 0.878 | 0.894 | 0.916 | 0.866 | 0.966 | 0.937 | 0.881 | 0.993 |
| RSAD2 IFI27 IFIT1 IFI44L SIGLEC1 IL1R2 | 0.886 | 0.878 | 0.894 | 0.919 | 0.868 | 0.969 | 0.946 | 0.897 | 0.995 |
| IFI27 OAS1 ISG15 HERC6 IL1R2 MMP8 | 0.886 | 0.878 | 0.894 | 0.918 | 0.870 | 0.967 | 0.928 | 0.868 | 0.988 |
| RSAD2 IFI27 OAS1 SIGLEC1 IL1R2 MMP8 | 0.886 | 0.877 | 0.894 | 0.919 | 0.871 | 0.968 | 0.957 | 0.912 | 1 |
| IFI27 OAS1 IFIT1 IFI44L ISG15 OLAH | 0.886 | 0.877 | 0.894 | 0.915 | 0.856 | 0.974 | 0.935 | 0.875 | 0.994 |
| IFIT1 SIGLEC1 HERC6 SLC1A2 OLAH FAM20A | 0.886 | 0.876 | 0.896 | 0.921 | 0.868 | 0.974 | 0.937 | 0.864 | 1 |
| RSAD2 IFI27 SLC1A2 IL1R2 FAM20A RETN | 0.886 | 0.877 | 0.895 | 0.929 | 0.880 | 0.978 | 0.925 | 0.838 | 1 |
| RSAD2 IFI27 IFI44L HERC6 IL1R2 MMP8 | 0.886 | 0.878 | 0.894 | 0.915 | 0.865 | 0.966 | 0.936 | 0.880 | 0.992 |
| RSAD2 IFI27 OAS1 IFIT1 SIGLEC1 IL1R2 | 0.886 | 0.878 | 0.894 | 0.920 | 0.871 | 0.968 | 0.958 | 0.914 | 1 |
| RSAD2 IFI27 IFIT1 ISG15 OLAH MMP8 | 0.886 | 0.877 | 0.895 | 0.915 | 0.856 | 0.973 | 0.935 | 0.873 | 0.996 |
| RSAD2 IFI27 OAS1 IFI44L OLAH MMP8 | 0.886 | 0.877 | 0.894 | 0.918 | 0.860 | 0.975 | 0.938 | 0.879 | 0.997 |
| OAS1 IFIT1 SIGLEC1 OLAH FAM20A MMP8 | 0.886 | 0.876 | 0.895 | 0.912 | 0.848 | 0.977 | 0.960 | 0.911 | 1 |
| IFI27 IFIT1 IFI44L HERC6 SLC1A2 OLAH | 0.886 | 0.877 | 0.895 | 0.928 | 0.877 | 0.978 | 0.934 | 0.872 | 0.995 |
| IFI27 OAS1 IFI44L ISG15 SLC1A2 OLAH | 0.886 | 0.877 | 0.895 | 0.927 | 0.876 | 0.979 | 0.924 | 0.853 | 0.995 |
| RSAD2 IFI27 IFI44L ISG15 OLAH MMP8 | 0.886 | 0.877 | 0.894 | 0.916 | 0.858 | 0.974 | 0.937 | 0.878 | 0.996 |
| IFI27 IFIT1 IFI44L HERC6 IL1R2 MMP8 | 0.886 | 0.878 | 0.894 | 0.918 | 0.868 | 0.969 | 0.936 | 0.880 | 0.992 |
| RSAD2 IFI27 OAS1 SLC1A2 OLAH RETN | 0.886 | 0.877 | 0.895 | 0.927 | 0.875 | 0.979 | 0.937 | 0.868 | 1 |
| RSAD2 IFI27 SIGLEC1 ISG15 IL1R2 RETN | 0.886 | 0.877 | 0.894 | 0.922 | 0.869 | 0.974 | 0.954 | 0.908 | 1 |
| IFI27 IFIT1 SIGLEC1 ISG15 IL1R2 MMP8 | 0.886 | 0.878 | 0.893 | 0.919 | 0.870 | 0.967 | 0.948 | 0.899 | 0.996 |
| RSAD2 IFIT1 SIGLEC1 IL1R2 OLAH FAM20A | 0.886 | 0.877 | 0.894 | 0.910 | 0.846 | 0.974 | 0.963 | 0.914 | 1 |
| IFI27 IFIT1 SIGLEC1 SLC1A2 IL1R2 RETN | 0.886 | 0.877 | 0.894 | 0.926 | 0.877 | 0.974 | 0.953 | 0.903 | 1 |
| RSAD2 IFI27 OAS1 IFI44L ISG15 OLAH | 0.886 | 0.877 | 0.894 | 0.917 | 0.859 | 0.975 | 0.935 | 0.876 | 0.994 |
| IFIT1 IFI44L SIGLEC1 IL1R2 OLAH FAM20A | 0.886 | 0.877 | 0.894 | 0.909 | 0.845 | 0.973 | 0.957 | 0.905 | 1 |
| IFI27 IFIT1 SLC1A2 IL1R2 FAM20A RETN | 0.886 | 0.877 | 0.895 | 0.927 | 0.877 | 0.977 | 0.929 | 0.847 | 1 |
| RSAD2 IFI27 OAS1 SIGLEC1 FAM20A MMP8 | 0.886 | 0.876 | 0.896 | 0.913 | 0.849 | 0.978 | 0.948 | 0.878 | 1 |
| RSAD2 IFI27 ISG15 SLC1A2 OLAH RETN | 0.886 | 0.877 | 0.894 | 0.928 | 0.877 | 0.979 | 0.933 | 0.866 | 1.000 |
| RSAD2 IFI27 OAS1 ISG15 OLAH RETN | 0.886 | 0.877 | 0.894 | 0.916 | 0.856 | 0.975 | 0.940 | 0.877 | 1 |
| RSAD2 IFI27 ISG15 SLC1A2 IL1R2 FAM20A | 0.886 | 0.877 | 0.894 | 0.927 | 0.879 | 0.975 | 0.925 | 0.839 | 1 |
| SIGLEC1 SLC1A2 IL1R2 FAM20A RETN MMP8 | 0.886 | 0.876 | 0.895 | 0.922 | 0.872 | 0.973 | 0.957 | 0.887 | 1 |
| IFI27 OAS1 ISG15 SLC1A2 OLAH RETN | 0.886 | 0.877 | 0.894 | 0.928 | 0.877 | 0.979 | 0.929 | 0.859 | 0.999 |
| IFI27 IFIT1 SIGLEC1 ISG15 SLC1A2 FAM20A | 0.886 | 0.877 | 0.895 | 0.922 | 0.866 | 0.977 | 0.942 | 0.867 | 1 |
| IFI27 IFI44L SIGLEC1 ISG15 SLC1A2 IL1R2 | 0.885 | 0.878 | 0.893 | 0.924 | 0.879 | 0.970 | 0.946 | 0.895 | 0.996 |
| RSAD2 IFI27 SIGLEC1 SLC1A2 FAM20A RETN | 0.885 | 0.876 | 0.895 | 0.923 | 0.866 | 0.979 | 0.945 | 0.871 | 1 |
| RSAD2 IFI27 IFIT1 IFI44L HERC6 IL1R2 | 0.885 | 0.878 | 0.893 | 0.918 | 0.868 | 0.969 | 0.938 | 0.883 | 0.993 |
| OAS1 IFIT1 SIGLEC1 ISG15 IL1R2 FAM20A | 0.885 | 0.876 | 0.894 | 0.913 | 0.850 | 0.976 | 0.961 | 0.910 | 1 |
| RSAD2 IFI27 IFIT1 ISG15 OLAH RETN | 0.885 | 0.877 | 0.894 | 0.916 | 0.857 | 0.975 | 0.940 | 0.878 | 1 |
| IFI27 OAS1 IFIT1 IFI44L SLC1A2 OLAH | 0.885 | 0.877 | 0.894 | 0.927 | 0.875 | 0.978 | 0.938 | 0.876 | 1 |
| RSAD2 OAS1 SIGLEC1 HERC6 IL1R2 FAM20A | 0.885 | 0.876 | 0.895 | 0.914 | 0.855 | 0.973 | 0.955 | 0.899 | 1 |
| IFI27 OAS1 IFIT1 IFI44L IL1R2 RETN | 0.885 | 0.877 | 0.894 | 0.918 | 0.865 | 0.972 | 0.941 | 0.883 | 0.999 |
| IFI27 IFI44L ISG15 IL1R2 RETN MMP8 | 0.885 | 0.877 | 0.894 | 0.922 | 0.869 | 0.974 | 0.940 | 0.880 | 1 |
| RSAD2 IFI27 IFIT1 IFI44L OLAH MMP8 | 0.885 | 0.877 | 0.894 | 0.915 | 0.856 | 0.973 | 0.941 | 0.884 | 0.999 |
| RSAD2 IFI27 OAS1 IFIT1 IL1R2 RETN | 0.885 | 0.877 | 0.894 | 0.916 | 0.862 | 0.971 | 0.950 | 0.897 | 1 |
| IFI27 IFIT1 IFI44L SLC1A2 OLAH RETN | 0.885 | 0.876 | 0.894 | 0.923 | 0.871 | 0.976 | 0.939 | 0.875 | 1 |
| IFI27 OAS1 IFIT1 SIGLEC1 ISG15 IL1R2 | 0.885 | 0.877 | 0.893 | 0.920 | 0.871 | 0.969 | 0.952 | 0.906 | 0.998 |
| IFI27 IFIT1 ISG15 SLC1A2 IL1R2 FAM20A | 0.885 | 0.876 | 0.894 | 0.923 | 0.873 | 0.974 | 0.925 | 0.840 | 1 |
| RSAD2 IFI27 OAS1 IFIT1 IFI44L OLAH | 0.885 | 0.877 | 0.894 | 0.916 | 0.858 | 0.974 | 0.939 | 0.881 | 0.998 |
| IFI27 OAS1 IFI44L SIGLEC1 ISG15 IL1R2 | 0.885 | 0.878 | 0.893 | 0.918 | 0.868 | 0.968 | 0.939 | 0.886 | 0.992 |
| IFI27 IFIT1 IFI44L SLC1A2 IL1R2 OLAH | 0.885 | 0.877 | 0.894 | 0.925 | 0.874 | 0.976 | 0.933 | 0.868 | 0.997 |
| RSAD2 IFI27 SIGLEC1 ISG15 FAM20A MMP8 | 0.885 | 0.876 | 0.895 | 0.915 | 0.851 | 0.979 | 0.946 | 0.875 | 1 |
| RSAD2 IFI27 SIGLEC1 SLC1A2 IL1R2 RETN | 0.885 | 0.877 | 0.894 | 0.923 | 0.874 | 0.973 | 0.953 | 0.903 | 1 |
| RSAD2 IFI27 OAS1 IFI44L SLC1A2 OLAH | 0.885 | 0.876 | 0.894 | 0.924 | 0.872 | 0.977 | 0.937 | 0.872 | 1 |
| IFI27 OAS1 IFIT1 ISG15 HERC6 IL1R2 | 0.885 | 0.877 | 0.893 | 0.918 | 0.869 | 0.966 | 0.939 | 0.884 | 0.995 |
| IFI27 IFI44L SIGLEC1 HERC6 RETN MMP8 | 0.885 | 0.876 | 0.895 | 0.912 | 0.851 | 0.974 | 0.942 | 0.885 | 1.000 |
| IFI27 IFI44L SIGLEC1 ISG15 FAM20A MMP8 | 0.885 | 0.876 | 0.894 | 0.910 | 0.844 | 0.976 | 0.943 | 0.869 | 1 |
| RSAD2 IFI27 OAS1 SLC1A2 IL1R2 FAM20A | 0.885 | 0.876 | 0.894 | 0.926 | 0.877 | 0.974 | 0.927 | 0.841 | 1 |
| RSAD2 IFIT1 SIGLEC1 HERC6 FAM20A RETN | 0.885 | 0.876 | 0.894 | 0.914 | 0.852 | 0.975 | 0.941 | 0.875 | 1 |
| RSAD2 IFI27 IFI44L SIGLEC1 ISG15 IL1R2 | 0.885 | 0.877 | 0.893 | 0.918 | 0.866 | 0.969 | 0.942 | 0.892 | 0.993 |
| IFI27 IFI44L ISG15 HERC6 IL1R2 MMP8 | 0.885 | 0.877 | 0.893 | 0.918 | 0.869 | 0.966 | 0.929 | 0.870 | 0.988 |
| IFI44L SIGLEC1 HERC6 IL1R2 OLAH FAM20A | 0.885 | 0.876 | 0.894 | 0.914 | 0.853 | 0.975 | 0.957 | 0.896 | 1 |
| RSAD2 IFI27 IFIT1 SLC1A2 OLAH RETN | 0.885 | 0.876 | 0.894 | 0.924 | 0.872 | 0.977 | 0.940 | 0.877 | 1 |
| RSAD2 IFI27 IFIT1 SIGLEC1 IL1R2 MMP8 | 0.885 | 0.877 | 0.893 | 0.920 | 0.872 | 0.968 | 0.950 | 0.903 | 0.997 |
| IFI27 IFIT1 IFI44L ISG15 IL1R2 RETN | 0.885 | 0.877 | 0.893 | 0.919 | 0.866 | 0.973 | 0.940 | 0.882 | 0.999 |
| IFI27 OAS1 SIGLEC1 ISG15 IL1R2 MMP8 | 0.885 | 0.877 | 0.893 | 0.919 | 0.871 | 0.967 | 0.949 | 0.901 | 0.997 |
| RSAD2 IFI27 IFI44L IL1R2 OLAH RETN | 0.885 | 0.876 | 0.894 | 0.918 | 0.861 | 0.975 | 0.942 | 0.883 | 1 |
| IFIT1 IFI44L SIGLEC1 SLC1A2 OLAH FAM20A | 0.885 | 0.876 | 0.894 | 0.920 | 0.864 | 0.975 | 0.934 | 0.868 | 1.000 |
| RSAD2 IFI27 OAS1 IFI44L OLAH RETN | 0.885 | 0.876 | 0.894 | 0.915 | 0.856 | 0.975 | 0.943 | 0.882 | 1 |
| RSAD2 IFI27 IFIT1 IL1R2 RETN MMP8 | 0.885 | 0.876 | 0.894 | 0.920 | 0.865 | 0.974 | 0.950 | 0.899 | 1 |
| IFI27 IFI44L SIGLEC1 SLC1A2 IL1R2 MMP8 | 0.885 | 0.877 | 0.893 | 0.925 | 0.879 | 0.970 | 0.948 | 0.900 | 0.996 |
| IFIT1 SIGLEC1 ISG15 HERC6 FAM20A MMP8 | 0.885 | 0.876 | 0.894 | 0.911 | 0.849 | 0.973 | 0.938 | 0.865 | 1 |
| IFI27 OAS1 ISG15 SLC1A2 IL1R2 FAM20A | 0.885 | 0.876 | 0.894 | 0.928 | 0.880 | 0.976 | 0.924 | 0.837 | 1 |
| RSAD2 IFI27 IFI44L SLC1A2 IL1R2 OLAH | 0.885 | 0.876 | 0.894 | 0.926 | 0.876 | 0.976 | 0.933 | 0.867 | 0.998 |
| IFI27 OAS1 HERC6 SLC1A2 FAM20A RETN | 0.885 | 0.875 | 0.894 | 0.923 | 0.868 | 0.979 | 0.932 | 0.849 | 1 |
| IFI27 IFI44L SIGLEC1 HERC6 FAM20A MMP8 | 0.885 | 0.875 | 0.894 | 0.912 | 0.849 | 0.974 | 0.941 | 0.862 | 1 |
| IFI27 SIGLEC1 ISG15 SLC1A2 IL1R2 RETN | 0.885 | 0.877 | 0.893 | 0.924 | 0.875 | 0.973 | 0.957 | 0.910 | 1 |
| IFI27 OAS1 IFI44L ISG15 OLAH RETN | 0.885 | 0.876 | 0.893 | 0.917 | 0.859 | 0.975 | 0.939 | 0.878 | 1 |
| RSAD2 OAS1 IFIT1 SIGLEC1 IL1R2 FAM20A | 0.885 | 0.876 | 0.894 | 0.912 | 0.849 | 0.975 | 0.965 | 0.917 | 1 |
| RSAD2 IFI27 OAS1 ISG15 HERC6 IL1R2 | 0.885 | 0.877 | 0.893 | 0.921 | 0.874 | 0.968 | 0.933 | 0.874 | 0.991 |
| IFI27 IFI44L SIGLEC1 SLC1A2 FAM20A RETN | 0.885 | 0.875 | 0.894 | 0.920 | 0.863 | 0.978 | 0.945 | 0.869 | 1 |
| IFIT1 IFI44L SIGLEC1 HERC6 FAM20A MMP8 | 0.885 | 0.875 | 0.894 | 0.911 | 0.850 | 0.973 | 0.935 | 0.864 | 1 |
| RSAD2 IFI27 IFIT1 IFI44L OLAH RETN | 0.885 | 0.876 | 0.893 | 0.916 | 0.857 | 0.975 | 0.945 | 0.884 | 1 |
| RSAD2 IFI27 IFIT1 ISG15 HERC6 IL1R2 | 0.885 | 0.877 | 0.892 | 0.918 | 0.869 | 0.967 | 0.937 | 0.882 | 0.992 |
| RSAD2 IFI27 SIGLEC1 ISG15 IL1R2 MMP8 | 0.885 | 0.877 | 0.893 | 0.919 | 0.871 | 0.968 | 0.948 | 0.899 | 0.996 |
| IFI27 SIGLEC1 ISG15 SLC1A2 FAM20A RETN | 0.885 | 0.875 | 0.894 | 0.922 | 0.865 | 0.978 | 0.943 | 0.866 | 1 |
| IFI44L SIGLEC1 SLC1A2 OLAH FAM20A MMP8 | 0.885 | 0.875 | 0.894 | 0.918 | 0.863 | 0.974 | 0.951 | 0.888 | 1 |
| RSAD2 SIGLEC1 ISG15 HERC6 IL1R2 FAM20A | 0.885 | 0.875 | 0.894 | 0.916 | 0.858 | 0.975 | 0.955 | 0.897 | 1 |
| IFIT1 IFI44L SIGLEC1 ISG15 IL1R2 FAM20A | 0.885 | 0.876 | 0.893 | 0.910 | 0.846 | 0.974 | 0.958 | 0.907 | 1 |
| RSAD2 IFI27 OAS1 SIGLEC1 SLC1A2 IL1R2 | 0.885 | 0.876 | 0.893 | 0.924 | 0.878 | 0.971 | 0.954 | 0.905 | 1 |
| OAS1 IFI44L SIGLEC1 HERC6 IL1R2 FAM20A | 0.885 | 0.876 | 0.893 | 0.915 | 0.857 | 0.974 | 0.959 | 0.905 | 1 |
| IFI27 OAS1 IFIT1 SIGLEC1 SLC1A2 IL1R2 | 0.884 | 0.876 | 0.893 | 0.926 | 0.880 | 0.971 | 0.951 | 0.902 | 1 |
| IFI27 OAS1 IFI44L SLC1A2 OLAH RETN | 0.884 | 0.875 | 0.893 | 0.928 | 0.877 | 0.979 | 0.935 | 0.866 | 1 |
| RSAD2 IFI27 IFI44L SLC1A2 OLAH RETN | 0.884 | 0.876 | 0.893 | 0.926 | 0.874 | 0.977 | 0.935 | 0.868 | 1 |
| IFI27 OAS1 SIGLEC1 ISG15 FAM20A MMP8 | 0.884 | 0.874 | 0.895 | 0.912 | 0.848 | 0.976 | 0.946 | 0.874 | 1 |
| RSAD2 IFI27 IFIT1 IFI44L SLC1A2 OLAH | 0.884 | 0.876 | 0.893 | 0.926 | 0.874 | 0.978 | 0.935 | 0.871 | 0.998 |
| IFI27 IFIT1 ISG15 IL1R2 RETN MMP8 | 0.884 | 0.876 | 0.893 | 0.918 | 0.863 | 0.974 | 0.949 | 0.897 | 1 |
| IFI27 ISG15 SLC1A2 IL1R2 FAM20A RETN | 0.884 | 0.876 | 0.893 | 0.924 | 0.874 | 0.974 | 0.912 | 0.819 | 1 |
| IFI27 OAS1 SLC1A2 IL1R2 FAM20A RETN | 0.884 | 0.876 | 0.893 | 0.929 | 0.880 | 0.978 | 0.924 | 0.837 | 1 |
| IFI27 IFIT1 SIGLEC1 SLC1A2 FAM20A MMP8 | 0.884 | 0.875 | 0.893 | 0.919 | 0.861 | 0.977 | 0.945 | 0.869 | 1 |
| IFI27 SIGLEC1 SLC1A2 IL1R2 RETN MMP8 | 0.884 | 0.876 | 0.893 | 0.926 | 0.878 | 0.974 | 0.966 | 0.927 | 1 |
| RSAD2 IFI44L SIGLEC1 HERC6 IL1R2 FAM20A | 0.884 | 0.875 | 0.893 | 0.915 | 0.856 | 0.974 | 0.958 | 0.901 | 1 |
| RSAD2 SIGLEC1 HERC6 IL1R2 OLAH FAM20A | 0.884 | 0.875 | 0.894 | 0.915 | 0.856 | 0.974 | 0.955 | 0.898 | 1 |
| IFI27 IFIT1 IFI44L ISG15 HERC6 IL1R2 | 0.884 | 0.876 | 0.892 | 0.919 | 0.869 | 0.969 | 0.936 | 0.879 | 0.993 |
| RSAD2 IFI27 IFIT1 ISG15 HERC6 RETN | 0.884 | 0.875 | 0.893 | 0.914 | 0.855 | 0.973 | 0.940 | 0.882 | 0.998 |
| RSAD2 IFI27 IFI44L ISG15 OLAH RETN | 0.884 | 0.876 | 0.893 | 0.916 | 0.857 | 0.976 | 0.938 | 0.874 | 1 |
| RSAD2 IFI27 OAS1 SIGLEC1 ISG15 IL1R2 | 0.884 | 0.876 | 0.892 | 0.921 | 0.873 | 0.969 | 0.955 | 0.911 | 1.000 |
| OAS1 IFIT1 SIGLEC1 IL1R2 FAM20A RETN | 0.884 | 0.875 | 0.893 | 0.912 | 0.849 | 0.974 | 0.961 | 0.908 | 1 |
| RSAD2 IFI27 SIGLEC1 SLC1A2 IL1R2 MMP8 | 0.884 | 0.876 | 0.892 | 0.924 | 0.878 | 0.970 | 0.953 | 0.906 | 1 |
| RSAD2 IFIT1 IFI44L SIGLEC1 IL1R2 FAM20A | 0.884 | 0.875 | 0.893 | 0.912 | 0.848 | 0.975 | 0.962 | 0.913 | 1 |
| RSAD2 IFI27 SIGLEC1 HERC6 SLC1A2 IL1R2 | 0.884 | 0.876 | 0.892 | 0.925 | 0.880 | 0.970 | 0.949 | 0.900 | 0.998 |
| RSAD2 IFI27 IFIT1 IFI44L IL1R2 RETN | 0.884 | 0.876 | 0.892 | 0.916 | 0.860 | 0.971 | 0.940 | 0.882 | 0.999 |
| IFI27 SIGLEC1 SLC1A2 FAM20A RETN MMP8 | 0.884 | 0.874 | 0.894 | 0.922 | 0.863 | 0.981 | 0.950 | 0.874 | 1 |
| RSAD2 IFI27 IFI44L SIGLEC1 FAM20A MMP8 | 0.884 | 0.874 | 0.893 | 0.913 | 0.848 | 0.978 | 0.948 | 0.878 | 1 |
| IFI27 OAS1 SIGLEC1 SLC1A2 IL1R2 MMP8 | 0.884 | 0.876 | 0.892 | 0.925 | 0.880 | 0.970 | 0.950 | 0.901 | 0.999 |
| RSAD2 SIGLEC1 SLC1A2 OLAH FAM20A MMP8 | 0.884 | 0.874 | 0.893 | 0.919 | 0.864 | 0.973 | 0.948 | 0.886 | 1 |
| IFI27 SIGLEC1 ISG15 SLC1A2 IL1R2 MMP8 | 0.884 | 0.876 | 0.892 | 0.924 | 0.879 | 0.970 | 0.950 | 0.901 | 0.999 |
| RSAD2 OAS1 IFIT1 SIGLEC1 HERC6 FAM20A | 0.884 | 0.875 | 0.893 | 0.911 | 0.850 | 0.972 | 0.937 | 0.865 | 1 |
| RSAD2 IFI27 IFIT1 IFI44L ISG15 OLAH | 0.884 | 0.875 | 0.892 | 0.915 | 0.857 | 0.974 | 0.936 | 0.876 | 0.996 |
| IFIT1 IFI44L SIGLEC1 IL1R2 FAM20A RETN | 0.884 | 0.875 | 0.893 | 0.909 | 0.847 | 0.972 | 0.960 | 0.908 | 1 |
| RSAD2 IFI27 OAS1 IL1R2 RETN MMP8 | 0.884 | 0.875 | 0.893 | 0.918 | 0.864 | 0.973 | 0.939 | 0.877 | 1 |
| IFI27 OAS1 IFI44L ISG15 HERC6 IL1R2 | 0.884 | 0.876 | 0.892 | 0.920 | 0.873 | 0.968 | 0.930 | 0.871 | 0.989 |
| RSAD2 IFI27 IFIT1 SIGLEC1 ISG15 IL1R2 | 0.884 | 0.876 | 0.892 | 0.920 | 0.871 | 0.968 | 0.947 | 0.897 | 0.996 |
| IFI27 OAS1 IFIT1 ISG15 IL1R2 RETN | 0.884 | 0.875 | 0.892 | 0.918 | 0.865 | 0.972 | 0.949 | 0.896 | 1 |
| SIGLEC1 ISG15 SLC1A2 OLAH FAM20A MMP8 | 0.884 | 0.874 | 0.893 | 0.916 | 0.860 | 0.971 | 0.951 | 0.889 | 1 |
| IFI27 IFIT1 SIGLEC1 ISG15 SLC1A2 IL1R2 | 0.884 | 0.876 | 0.892 | 0.924 | 0.878 | 0.969 | 0.949 | 0.900 | 0.998 |
| OAS1 IFIT1 IFI44L SIGLEC1 HERC6 FAM20A | 0.884 | 0.875 | 0.892 | 0.909 | 0.847 | 0.971 | 0.936 | 0.866 | 1 |
| RSAD2 IFIT1 SIGLEC1 ISG15 HERC6 FAM20A | 0.884 | 0.875 | 0.892 | 0.911 | 0.850 | 0.972 | 0.936 | 0.862 | 1 |
| RSAD2 IFIT1 SIGLEC1 ISG15 IL1R2 FAM20A | 0.884 | 0.875 | 0.892 | 0.911 | 0.847 | 0.975 | 0.963 | 0.914 | 1 |
| RSAD2 IFI27 IFI44L ISG15 HERC6 RETN | 0.883 | 0.875 | 0.892 | 0.914 | 0.855 | 0.973 | 0.941 | 0.884 | 0.998 |
| IFI27 OAS1 IFI44L SLC1A2 IL1R2 OLAH | 0.883 | 0.874 | 0.893 | 0.927 | 0.878 | 0.977 | 0.929 | 0.861 | 0.998 |
| IFI27 IFIT1 SIGLEC1 SLC1A2 IL1R2 MMP8 | 0.883 | 0.875 | 0.892 | 0.924 | 0.878 | 0.970 | 0.955 | 0.911 | 1 |
| OAS1 IFIT1 IFI44L SIGLEC1 OLAH FAM20A | 0.883 | 0.874 | 0.893 | 0.905 | 0.835 | 0.974 | 0.948 | 0.893 | 1 |
| IFI27 OAS1 IFIT1 SIGLEC1 SLC1A2 FAM20A | 0.883 | 0.874 | 0.893 | 0.920 | 0.863 | 0.977 | 0.943 | 0.871 | 1 |
| IFI27 IFIT1 SIGLEC1 HERC6 SLC1A2 IL1R2 | 0.883 | 0.875 | 0.892 | 0.925 | 0.880 | 0.971 | 0.951 | 0.904 | 0.998 |
| OAS1 IFIT1 SIGLEC1 HERC6 FAM20A MMP8 | 0.883 | 0.874 | 0.893 | 0.910 | 0.848 | 0.972 | 0.938 | 0.868 | 1 |
| IFIT1 SIGLEC1 ISG15 IL1R2 OLAH FAM20A | 0.883 | 0.874 | 0.892 | 0.913 | 0.850 | 0.975 | 0.962 | 0.914 | 1 |
| RSAD2 IFI27 HERC6 SLC1A2 FAM20A RETN | 0.883 | 0.874 | 0.893 | 0.921 | 0.865 | 0.977 | 0.932 | 0.849 | 1 |
| RSAD2 IFI27 OAS1 IFI44L IL1R2 RETN | 0.883 | 0.875 | 0.892 | 0.916 | 0.862 | 0.971 | 0.941 | 0.883 | 1.000 |
| SIGLEC1 ISG15 HERC6 IL1R2 OLAH FAM20A | 0.883 | 0.874 | 0.893 | 0.911 | 0.849 | 0.974 | 0.958 | 0.899 | 1 |
| RSAD2 IFI27 IFI44L ISG15 HERC6 IL1R2 | 0.883 | 0.875 | 0.891 | 0.919 | 0.870 | 0.968 | 0.932 | 0.874 | 0.989 |
| RSAD2 IFI27 IFIT1 IFI44L HERC6 RETN | 0.883 | 0.874 | 0.892 | 0.912 | 0.852 | 0.973 | 0.940 | 0.879 | 1 |
| RSAD2 IFI27 OAS1 IFIT1 IFI44L IL1R2 | 0.883 | 0.875 | 0.891 | 0.915 | 0.863 | 0.968 | 0.940 | 0.886 | 0.994 |
| IFI27 IFI44L HERC6 SLC1A2 IL1R2 RETN | 0.883 | 0.874 | 0.892 | 0.923 | 0.874 | 0.973 | 0.934 | 0.868 | 0.999 |
| OAS1 IFIT1 SIGLEC1 ISG15 HERC6 FAM20A | 0.883 | 0.874 | 0.892 | 0.910 | 0.849 | 0.971 | 0.941 | 0.873 | 1 |
| OAS1 SIGLEC1 HERC6 IL1R2 OLAH FAM20A | 0.883 | 0.874 | 0.893 | 0.913 | 0.853 | 0.972 | 0.959 | 0.905 | 1 |
| RSAD2 IFI27 IFIT1 SIGLEC1 SLC1A2 FAM20A | 0.883 | 0.874 | 0.892 | 0.921 | 0.865 | 0.977 | 0.945 | 0.869 | 1 |
| RSAD2 IFI27 OAS1 IFIT1 IL1R2 MMP8 | 0.883 | 0.875 | 0.892 | 0.915 | 0.864 | 0.965 | 0.943 | 0.891 | 0.996 |
| IFIT1 SIGLEC1 ISG15 OLAH FAM20A MMP8 | 0.883 | 0.874 | 0.893 | 0.907 | 0.841 | 0.972 | 0.957 | 0.909 | 1 |
| RSAD2 IFI27 HERC6 SLC1A2 IL1R2 RETN | 0.883 | 0.875 | 0.892 | 0.923 | 0.874 | 0.973 | 0.935 | 0.871 | 0.999 |
| OAS1 SIGLEC1 SLC1A2 OLAH FAM20A MMP8 | 0.883 | 0.873 | 0.893 | 0.917 | 0.861 | 0.972 | 0.951 | 0.890 | 1 |
| RSAD2 IFI27 ISG15 HERC6 IL1R2 MMP8 | 0.883 | 0.875 | 0.891 | 0.916 | 0.866 | 0.965 | 0.933 | 0.875 | 0.990 |
| OAS1 IFIT1 SIGLEC1 ISG15 OLAH FAM20A | 0.883 | 0.874 | 0.892 | 0.905 | 0.836 | 0.974 | 0.949 | 0.896 | 1 |
| OAS1 SIGLEC1 ISG15 HERC6 IL1R2 FAM20A | 0.883 | 0.874 | 0.892 | 0.914 | 0.855 | 0.972 | 0.959 | 0.905 | 1 |
| IFI27 OAS1 SIGLEC1 HERC6 SLC1A2 FAM20A | 0.883 | 0.873 | 0.893 | 0.921 | 0.866 | 0.975 | 0.945 | 0.869 | 1 |
| IFI27 OAS1 IFI44L IL1R2 RETN MMP8 | 0.883 | 0.875 | 0.891 | 0.918 | 0.864 | 0.972 | 0.939 | 0.879 | 0.999 |
| IFI27 IFIT1 ISG15 HERC6 IL1R2 MMP8 | 0.883 | 0.875 | 0.891 | 0.915 | 0.865 | 0.965 | 0.938 | 0.884 | 0.993 |
| RSAD2 IFIT1 SIGLEC1 OLAH FAM20A MMP8 | 0.883 | 0.874 | 0.892 | 0.908 | 0.843 | 0.974 | 0.959 | 0.911 | 1 |
| IFI27 OAS1 IFI44L SIGLEC1 FAM20A MMP8 | 0.883 | 0.873 | 0.893 | 0.912 | 0.847 | 0.976 | 0.946 | 0.875 | 1 |
| IFI27 OAS1 SIGLEC1 ISG15 SLC1A2 IL1R2 | 0.883 | 0.875 | 0.891 | 0.925 | 0.880 | 0.970 | 0.950 | 0.901 | 0.999 |
| IFI27 OAS1 ISG15 HERC6 SLC1A2 RETN | 0.883 | 0.874 | 0.892 | 0.917 | 0.860 | 0.974 | 0.932 | 0.859 | 1 |
| RSAD2 IFI27 SIGLEC1 HERC6 SLC1A2 RETN | 0.883 | 0.874 | 0.892 | 0.918 | 0.863 | 0.973 | 0.948 | 0.891 | 1 |
| IFIT1 IFI44L SIGLEC1 ISG15 OLAH FAM20A | 0.883 | 0.874 | 0.892 | 0.905 | 0.836 | 0.974 | 0.945 | 0.888 | 1 |
| IFIT1 IFI44L SIGLEC1 HERC6 SLC1A2 FAM20A | 0.883 | 0.874 | 0.892 | 0.919 | 0.866 | 0.972 | 0.934 | 0.856 | 1 |
| OAS1 IFIT1 SIGLEC1 SLC1A2 OLAH FAM20A | 0.883 | 0.874 | 0.892 | 0.919 | 0.863 | 0.974 | 0.945 | 0.881 | 1 |
| IFI44L SIGLEC1 ISG15 IL1R2 OLAH FAM20A | 0.883 | 0.874 | 0.892 | 0.910 | 0.848 | 0.973 | 0.961 | 0.909 | 1 |
| RSAD2 IFIT1 IFI44L SIGLEC1 HERC6 FAM20A | 0.883 | 0.874 | 0.891 | 0.910 | 0.849 | 0.971 | 0.932 | 0.860 | 1 |
| RSAD2 IFI27 OAS1 HERC6 SLC1A2 RETN | 0.883 | 0.873 | 0.892 | 0.914 | 0.854 | 0.974 | 0.929 | 0.851 | 1 |
| IFI27 IFI44L SIGLEC1 HERC6 SLC1A2 IL1R2 | 0.883 | 0.874 | 0.891 | 0.927 | 0.883 | 0.972 | 0.946 | 0.896 | 0.995 |
| RSAD2 IFI27 SIGLEC1 ISG15 SLC1A2 IL1R2 | 0.883 | 0.875 | 0.891 | 0.924 | 0.879 | 0.970 | 0.949 | 0.900 | 0.998 |
| IFIT1 SIGLEC1 IL1R2 OLAH FAM20A RETN | 0.883 | 0.874 | 0.892 | 0.910 | 0.847 | 0.972 | 0.960 | 0.908 | 1 |
| IFIT1 SIGLEC1 HERC6 SLC1A2 FAM20A RETN | 0.883 | 0.873 | 0.892 | 0.919 | 0.865 | 0.972 | 0.936 | 0.859 | 1 |
| RSAD2 IFI27 SIGLEC1 HERC6 SLC1A2 FAM20A | 0.883 | 0.873 | 0.892 | 0.921 | 0.866 | 0.976 | 0.940 | 0.864 | 1 |
| RSAD2 IFI27 IFIT1 IFI44L IL1R2 MMP8 | 0.883 | 0.875 | 0.890 | 0.915 | 0.863 | 0.968 | 0.937 | 0.881 | 0.993 |
| IFI27 IFIT1 HERC6 SLC1A2 FAM20A RETN | 0.883 | 0.873 | 0.892 | 0.919 | 0.861 | 0.976 | 0.933 | 0.851 | 1 |
| RSAD2 IFI27 IFIT1 ISG15 IL1R2 RETN | 0.882 | 0.874 | 0.891 | 0.917 | 0.864 | 0.971 | 0.947 | 0.894 | 1.000 |
| IFI27 SIGLEC1 HERC6 SLC1A2 IL1R2 RETN | 0.882 | 0.874 | 0.891 | 0.924 | 0.874 | 0.973 | 0.954 | 0.906 | 1 |
| IFIT1 IFI44L SIGLEC1 SLC1A2 IL1R2 FAM20A | 0.882 | 0.874 | 0.891 | 0.920 | 0.869 | 0.972 | 0.948 | 0.888 | 1 |
| IFI27 IFIT1 IFI44L ISG15 HERC6 RETN | 0.882 | 0.873 | 0.892 | 0.911 | 0.849 | 0.972 | 0.939 | 0.874 | 1 |
| RSAD2 IFI27 IFI44L IL1R2 RETN MMP8 | 0.882 | 0.874 | 0.891 | 0.919 | 0.864 | 0.973 | 0.943 | 0.887 | 1.000 |
| IFI27 SIGLEC1 HERC6 SLC1A2 FAM20A RETN | 0.882 | 0.872 | 0.892 | 0.920 | 0.862 | 0.978 | 0.949 | 0.873 | 1 |
| IFI44L SIGLEC1 HERC6 IL1R2 FAM20A RETN | 0.882 | 0.873 | 0.892 | 0.912 | 0.852 | 0.973 | 0.955 | 0.893 | 1 |
| IFI27 IFIT1 IFI44L SIGLEC1 SLC1A2 IL1R2 | 0.882 | 0.874 | 0.891 | 0.924 | 0.877 | 0.970 | 0.950 | 0.903 | 0.997 |
| RSAD2 IFIT1 SIGLEC1 HERC6 FAM20A MMP8 | 0.882 | 0.873 | 0.892 | 0.911 | 0.849 | 0.972 | 0.937 | 0.865 | 1 |
| IFI27 OAS1 IFIT1 IFI44L IL1R2 MMP8 | 0.882 | 0.874 | 0.890 | 0.917 | 0.865 | 0.969 | 0.939 | 0.885 | 0.993 |
| IFI27 IFIT1 SIGLEC1 HERC6 SLC1A2 RETN | 0.882 | 0.873 | 0.891 | 0.919 | 0.864 | 0.973 | 0.950 | 0.897 | 1 |
| RSAD2 OAS1 IFIT1 SIGLEC1 OLAH FAM20A | 0.882 | 0.873 | 0.892 | 0.905 | 0.836 | 0.975 | 0.955 | 0.906 | 1 |
| IFI27 OAS1 IFI44L HERC6 SLC1A2 RETN | 0.882 | 0.873 | 0.891 | 0.914 | 0.855 | 0.973 | 0.930 | 0.857 | 1 |
| IFI27 OAS1 IFIT1 SIGLEC1 ISG15 RETN | 0.882 | 0.873 | 0.891 | 0.909 | 0.848 | 0.970 | 0.954 | 0.896 | 1 |
| IFI27 SIGLEC1 ISG15 HERC6 FAM20A MMP8 | 0.882 | 0.872 | 0.892 | 0.905 | 0.837 | 0.972 | 0.942 | 0.863 | 1 |
| SIGLEC1 HERC6 SLC1A2 OLAH FAM20A MMP8 | 0.882 | 0.872 | 0.892 | 0.918 | 0.863 | 0.973 | 0.951 | 0.883 | 1 |
| RSAD2 IFI27 ISG15 HERC6 RETN MMP8 | 0.882 | 0.873 | 0.891 | 0.914 | 0.855 | 0.973 | 0.940 | 0.883 | 0.998 |
| RSAD2 OAS1 SIGLEC1 IL1R2 OLAH FAM20A | 0.882 | 0.873 | 0.891 | 0.910 | 0.847 | 0.973 | 0.963 | 0.912 | 1 |
| RSAD2 IFI27 IFIT1 SIGLEC1 SLC1A2 IL1R2 | 0.882 | 0.874 | 0.890 | 0.924 | 0.878 | 0.90 | 0.952 | 0.905 | 1.000 |
| RSAD2 IFI27 OAS1 IFIT1 SIGLEC1 RETN | 0.882 | 0.873 | 0.891 | 0.909 | 0.846 | 0.971 | 0.952 | 0.892 | 1 |
| IFI27 IFI44L SLC1A2 IL1R2 RETN MMP8 | 0.882 | 0.873 | 0.891 | 0.924 | 0.875 | 0.972 | 0.936 | 0.869 | 1 |
| RSAD2 IFIT1 SIGLEC1 SLC1A2 IL1R2 FAM20A | 0.882 | 0.873 | 0.891 | 0.917 | 0.862 | 0.972 | 0.954 | 0.892 | 1 |
| RSAD2 SIGLEC1 HERC6 SLC1A2 IL1R2 FAM20A | 0.882 | 0.872 | 0.892 | 0.919 | 0.867 | 0.971 | 0.947 | 0.874 | 1 |
| IFIT1 SIGLEC1 SLC1A2 IL1R2 OLAH FAM20A | 0.882 | 0.873 | 0.891 | 0.916 | 0.862 | 0.971 | 0.952 | 0.894 | 1 |
| IFI27 IFIT1 IFI44L ISG15 IL1R2 MMP8 | 0.882 | 0.873 | 0.890 | 0.918 | 0.866 | 0.969 | 0.937 | 0.881 | 0.993 |
| RSAD2 IFIT1 SIGLEC1 IL1R2 FAM20A RETN | 0.882 | 0.873 | 0.891 | 0.911 | 0.847 | 0.974 | 0.963 | 0.913 | 1 |
| RSAD2 SIGLEC1 HERC6 IL1R2 FAM20A RETN | 0.882 | 0.872 | 0.891 | 0.913 | 0.854 | 0.973 | 0.955 | 0.894 | 1 |
| IFIT1 SIGLEC1 HERC6 SLC1A2 FAM20A MMP8 | 0.882 | 0.872 | 0.891 | 0.917 | 0.862 | 0.971 | 0.940 | 0.864 | 1 |
| IFIT1 SIGLEC1 ISG15 IL1R2 FAM20A RETN | 0.882 | 0.873 | 0.891 | 0.909 | 0.846 | 0.972 | 0.961 | 0.910 | 1 |
| RSAD2 IFIT1 SIGLEC1 SLC1A2 OLAH FAM20A | 0.882 | 0.873 | 0.891 | 0.920 | 0.865 | 0.975 | 0.950 | 0.890 | 1 |
| OAS1 IFIT1 SIGLEC1 SLC1A2 IL1R2 FAM20A | 0.882 | 0.873 | 0.891 | 0.914 | 0.857 | 0.971 | 0.951 | 0.888 | 1 |
| OAS1 SIGLEC1 HERC6 SLC1A2 IL1R2 FAM20A | 0.882 | 0.872 | 0.891 | 0.919 | 0.868 | 0.970 | 0.949 | 0.878 | 1 |
| IFI27 IFIT1 IFI44L HERC6 FAM20A MMP8 | 0.882 | 0.872 | 0.891 | 0.910 | 0.849 | 0.971 | 0.924 | 0.842 | 1 |
| IFI27 SIGLEC1 ISG15 HERC6 SLC1A2 IL1R2 | 0.882 | 0.873 | 0.890 | 0.924 | 0.879 | 0.970 | 0.949 | 0.900 | 0.998 |
| IFI27 OAS1 IFIT1 HERC6 SLC1A2 RETN | 0.882 | 0.872 | 0.891 | 0.913 | 0.854 | 0.972 | 0.936 | 0.863 | 1 |
| IFI27 OAS1 IFI44L ISG15 IL1R2 RETN | 0.882 | 0.873 | 0.890 | 0.919 | 0.867 | 0.972 | 0.938 | 0.878 | 0.998 |
| IFI27 IFIT1 IFI44L ISG15 FAM20A RETN | 0.882 | 0.872 | 0.891 | 0.910 | 0.846 | 0.974 | 0.943 | 0.866 | 1 |
| IFI27 OAS1 IFI44L SIGLEC1 SLC1A2 IL1R2 | 0.882 | 0.873 | 0.890 | 0.923 | 0.877 | 0.969 | 0.945 | 0.893 | 0.996 |
| IFI44L SIGLEC1 HERC6 SLC1A2 IL1R2 FAM20A | 0.882 | 0.872 | 0.891 | 0.918 | 0.865 | 0.972 | 0.951 | 0.879 | 1 |
| IFIT1 SIGLEC1 OLAH FAM20A RETN MMP8 | 0.882 | 0.872 | 0.891 | 0.912 | 0.850 | 0.975 | 0.958 | 0.909 | 1 |
| OAS1 IFI44L SIGLEC1 IL1R2 OLAH FAM20A | 0.881 | 0.872 | 0.891 | 0.910 | 0.847 | 0.973 | 0.963 | 0.912 | 1 |
| IFI44L SIGLEC1 SLC1A2 IL1R2 OLAH FAM20A | 0.881 | 0.873 | 0.890 | 0.916 | 0.861 | 0.971 | 0.954 | 0.893 | 1 |
| IFI27 OAS1 IFIT1 IFI44L SIGLEC1 RETN | 0.881 | 0.872 | 0.890 | 0.910 | 0.849 | 0.970 | 0.955 | 0.897 | 1 |
| IFI27 ISG15 HERC6 SLC1A2 FAM20A RETN | 0.881 | 0.872 | 0.891 | 0.918 | 0.861 | 0.976 | 0.941 | 0.862 | 1 |
| IFI27 OAS1 IFIT1 SLC1A2 IL1R2 RETN | 0.881 | 0.873 | 0.890 | 0.920 | 0.868 | 0.971 | 0.941 | 0.878 | 1 |
| IFIT1 SIGLEC1 ISG15 HERC6 SLC1A2 FAM20A | 0.881 | 0.872 | 0.891 | 0.918 | 0.864 | 0.972 | 0.940 | 0.864 | 1 |
| OAS1 IFIT1 SIGLEC1 OLAH FAM20A RETN | 0.881 | 0.872 | 0.891 | 0.905 | 0.835 | 0.974 | 0.955 | 0.906 | 1 |
| IFI27 SIGLEC1 HERC6 SLC1A2 IL1R2 MMP8 | 0.881 | 0.873 | 0.890 | 0.924 | 0.878 | 0.970 | 0.955 | 0.911 | 1 |
| IFI27 OAS1 IFIT1 HERC6 SLC1A2 IL1R2 | 0.881 | 0.873 | 0.890 | 0.922 | 0.876 | 0.968 | 0.937 | 0.874 | 1 |
| IFI27 IFI44L HERC6 SLC1A2 FAM20A RETN | 0.881 | 0.872 | 0.891 | 0.919 | 0.862 | 0.976 | 0.941 | 0.862 | 1 |
| RSAD2 IFI27 IFI44L ISG15 IL1R2 RETN | 0.881 | 0.873 | 0.889 | 0.918 | 0.864 | 0.972 | 0.938 | 0.878 | 0.998 |
| IFI27 IFIT1 HERC6 SLC1A2 IL1R2 RETN | 0.881 | 0.873 | 0.890 | 0.922 | 0.872 | 0.972 | 0.936 | 0.872 | 1.000 |
| SIGLEC1 ISG15 IL1R2 OLAH FAM20A RETN | 0.881 | 0.872 | 0.890 | 0.906 | 0.841 | 0.970 | 0.963 | 0.910 | 1 |
| OAS1 SIGLEC1 HERC6 IL1R2 FAM20A RETN | 0.881 | 0.872 | 0.890 | 0.912 | 0.852 | 0.971 | 0.960 | 0.905 | 1 |
| IFI44L SIGLEC1 ISG15 SLC1A2 IL1R2 FAM20A | 0.881 | 0.872 | 0.890 | 0.915 | 0.860 | 0.969 | 0.953 | 0.888 | 1 |
| IFI27 OAS1 ISG15 IL1R2 RETN MMP8 | 0.881 | 0.873 | 0.889 | 0.919 | 0.866 | 0.972 | 0.940 | 0.881 | 0.999 |
| RSAD2 IFI27 ISG15 IL1R2 RETN MMP8 | 0.881 | 0.872 | 0.890 | 0.919 | 0.864 | 0.973 | 0.942 | 0.884 | 1 |
| IFI27 OAS1 IFIT1 SIGLEC1 SLC1A2 RETN | 0.881 | 0.872 | 0.890 | 0.915 | 0.859 | 0.971 | 0.947 | 0.881 | 1 |
| IFI27 IFIT1 IFI44L HERC6 RETN MMP8 | 0.881 | 0.871 | 0.891 | 0.908 | 0.846 | 0.971 | 0.929 | 0.859 | 0.999 |
| IFI27 OAS1 IFIT1 IFI44L ISG15 IL1R2 | 0.881 | 0.873 | 0.889 | 0.916 | 0.864 | 0.968 | 0.939 | 0.885 | 0.994 |
| IFI27 IFI44L ISG15 SLC1A2 IL1R2 RETN | 0.881 | 0.873 | 0.889 | 0.922 | 0.872 | 0.972 | 0.933 | 0.866 | 0.999 |
| RSAD2 IFI27 OAS1 IFI44L IL1R2 MMP8 | 0.881 | 0.873 | 0.889 | 0.914 | 0.862 | 0.966 | 0.937 | 0.881 | 0.993 |
| IFI27 IFI44L ISG15 FAM20A RETN MMP8 | 0.881 | 0.870 | 0.891 | 0.912 | 0.849 | 0.975 | 0.948 | 0.874 | 1 |
| IFI27 IFIT1 SLC1A2 IL1R2 RETN MMP8 | 0.881 | 0.872 | 0.890 | 0.922 | 0.872 | 0.973 | 0.945 | 0.886 | 1 |
| IFI44L SIGLEC1 ISG15 IL1R2 FAM20A RETN | 0.881 | 0.872 | 0.890 | 0.907 | 0.842 | 0.971 | 0.962 | 0.909 | 1 |
| RSAD2 SIGLEC1 SLC1A2 IL1R2 OLAH FAM20A | 0.881 | 0.871 | 0.890 | 0.915 | 0.860 | 0.970 | 0.954 | 0.893 | 1 |
| IFI27 OAS1 SIGLEC1 ISG15 SLC1A2 FAM20A | 0.881 | 0.871 | 0.891 | 0.916 | 0.857 | 0.974 | 0.946 | 0.872 | 1 |
| IFI27 HERC6 SLC1A2 IL1R2 RETN MMP8 | 0.881 | 0.872 | 0.890 | 0.923 | 0.873 | 0.973 | 0.935 | 0.868 | 1 |
| RSAD2 IFI27 OAS1 HERC6 SLC1A2 IL1R2 | 0.881 | 0.872 | 0.889 | 0.923 | 0.878 | 0.968 | 0.932 | 0.866 | 0.997 |
| RSAD2 IFI27 OAS1 ISG15 IL1R2 RETN | 0.881 | 0.872 | 0.889 | 0.917 | 0.863 | 0.971 | 0.945 | 0.888 | 1 |
| IFI44L SIGLEC1 IL1R2 OLAH FAM20A RETN | 0.881 | 0.871 | 0.890 | 0.907 | 0.843 | 0.971 | 0.963 | 0.912 | 1 |
| SIGLEC1 HERC6 SLC1A2 IL1R2 OLAH FAM20A | 0.881 | 0.871 | 0.890 | 0.919 | 0.865 | 0.972 | 0.947 | 0.874 | 1 |
| IFIT1 IFI44L SIGLEC1 OLAH FAM20A RETN | 0.881 | 0.871 | 0.890 | 0.905 | 0.836 | 0.973 | 0.943 | 0.887 | 1 |
| IFI27 ISG15 HERC6 SLC1A2 IL1R2 RETN | 0.881 | 0.872 | 0.889 | 0.923 | 0.873 | 0.973 | 0.936 | 0.872 | 1.000 |
| RSAD2 IFI27 OAS1 SIGLEC1 SLC1A2 FAM20A | 0.881 | 0.871 | 0.890 | 0.919 | 0.860 | 0.977 | 0.949 | 0.878 | 1 |
| RSAD2 IFIT1 IFI44L SIGLEC1 OLAH FAM20A | 0.881 | 0.871 | 0.890 | 0.905 | 0.836 | 0.974 | 0.947 | 0.892 | 1 |
| RSAD2 IFI27 OAS1 IFI44L SIGLEC1 RETN | 0.880 | 0.872 | 0.889 | 0.908 | 0.846 | 0.971 | 0.952 | 0.889 | 1 |
| RSAD2 IFI27 SIGLEC1 ISG15 SLC1A2 FAM20A | 0.880 | 0.871 | 0.890 | 0.917 | 0.858 | 0.976 | 0.946 | 0.872 | 1 |
| RSAD2 IFI27 SIGLEC1 SLC1A2 FAM20A MMP8 | 0.880 | 0.871 | 0.890 | 0.919 | 0.860 | 0.978 | 0.947 | 0.873 | 1 |
| IFI27 OAS1 SIGLEC1 SLC1A2 FAM20A MMP8 | 0.880 | 0.871 | 0.890 | 0.918 | 0.859 | 0.976 | 0.945 | 0.870 | 1 |
| IFI27 OAS1 IFI44L SIGLEC1 ISG15 RETN | 0.880 | 0.872 | 0.889 | 0.910 | 0.848 | 0.971 | 0.954 | 0.894 | 1 |
| OAS1 SIGLEC1 ISG15 IL1R2 OLAH FAM20A | 0.880 | 0.871 | 0.890 | 0.909 | 0.845 | 0.973 | 0.963 | 0.912 | 1 |
| RSAD2 IFI27 IFI44L ISG15 HERC6 FAM20A | 0.880 | 0.871 | 0.890 | 0.913 | 0.851 | 0.974 | 0.926 | 0.845 | 1 |
| IFI27 IFIT1 IFI44L SLC1A2 IL1R2 RETN | 0.880 | 0.872 | 0.889 | 0.919 | 0.866 | 0.972 | 0.938 | 0.875 | 1 |
| RSAD2 OAS1 IFI44L SIGLEC1 IL1R2 FAM20A | 0.880 | 0.871 | 0.890 | 0.910 | 0.846 | 0.973 | 0.963 | 0.912 | 1 |
| IFI27 IFIT1 IFI44L SIGLEC1 ISG15 RETN | 0.880 | 0.871 | 0.890 | 0.908 | 0.846 | 0.970 | 0.950 | 0.892 | 1 |
| RSAD2 IFI27 IFI44L SIGLEC1 SLC1A2 IL1R2 | 0.880 | 0.872 | 0.889 | 0.925 | 0.878 | 0.971 | 0.951 | 0.905 | 0.997 |
| RSAD2 IFI44L SIGLEC1 IL1R2 OLAH FAM20A | 0.880 | 0.871 | 0.889 | 0.910 | 0.847 | 0.973 | 0.964 | 0.914 | 1 |
| RSAD2 IFIT1 SIGLEC1 HERC6 SLC1A2 FAM20A | 0.880 | 0.871 | 0.889 | 0.917 | 0.863 | 0.971 | 0.937 | 0.860 | 1 |
| RSAD2 OAS1 SIGLEC1 IL1R2 FAM20A RETN | 0.880 | 0.871 | 0.890 | 0.908 | 0.844 | 0.972 | 0.961 | 0.906 | 1 |
| OAS1 SIGLEC1 SLC1A2 IL1R2 OLAH FAM20A | 0.880 | 0.871 | 0.890 | 0.915 | 0.860 | 0.970 | 0.955 | 0.896 | 1 |
| IFI27 IFIT1 ISG15 HERC6 RETN MMP8 | 0.880 | 0.871 | 0.890 | 0.909 | 0.848 | 0.971 | 0.935 | 0.870 | 1.000 |
| RSAD2 IFI27 IFI44L HERC6 RETN MMP8 | 0.880 | 0.871 | 0.889 | 0.911 | 0.850 | 0.972 | 0.933 | 0.868 | 0.997 |
| RSAD2 IFI27 IFIT1 HERC6 RETN MMP8 | 0.880 | 0.871 | 0.890 | 0.911 | 0.850 | 0.972 | 0.933 | 0.868 | 0.997 |
| OAS1 IFI44L SIGLEC1 ISG15 IL1R2 FAM20A | 0.880 | 0.871 | 0.889 | 0.909 | 0.845 | 0.973 | 0.962 | 0.911 | 1 |
| SIGLEC1 SLC1A2 IL1R2 OLAH FAM20A RETN | 0.880 | 0.871 | 0.889 | 0.916 | 0.863 | 0.970 | 0.957 | 0.896 | 1 |
| IFI27 OAS1 IFIT1 SIGLEC1 RETN MMP8 | 0.880 | 0.871 | 0.890 | 0.910 | 0.849 | 0.971 | 0.955 | 0.897 | 1 |
| IFI44L SIGLEC1 SLC1A2 IL1R2 FAM20A RETN | 0.880 | 0.871 | 0.889 | 0.915 | 0.862 | 0.969 | 0.953 | 0.885 | 1 |
| SIGLEC1 SLC1A2 OLAH FAM20A RETN MMP8 | 0.880 | 0.870 | 0.890 | 0.916 | 0.858 | 0.973 | 0.948 | 0.879 | 1 |
| RSAD2 IFI27 IFIT1 ISG15 IL1R2 MMP8 | 0.880 | 0.872 | 0.888 | 0.915 | 0.864 | 0.966 | 0.942 | 0.890 | 0.995 |
| RSAD2 IFI27 IFIT1 IFI44L ISG15 IL1R2 | 0.880 | 0.872 | 0.888 | 0.917 | 0.865 | 0.969 | 0.939 | 0.885 | 0.994 |
| IFIT1 SIGLEC1 ISG15 SLC1A2 OLAH FAM20A | 0.880 | 0.871 | 0.889 | 0.918 | 0.863 | 0.973 | 0.943 | 0.882 | 1 |
| IFI44L SIGLEC1 ISG15 SLC1A2 OLAH FAM20A | 0.880 | 0.871 | 0.889 | 0.911 | 0.852 | 0.970 | 0.945 | 0.878 | 1 |
| SIGLEC1 ISG15 SLC1A2 IL1R2 OLAH FAM20A | 0.880 | 0.871 | 0.889 | 0.915 | 0.861 | 0.970 | 0.954 | 0.893 | 1 |
| RSAD2 OAS1 SIGLEC1 ISG15 IL1R2 FAM20A | 0.880 | 0.871 | 0.889 | 0.910 | 0.846 | 0.973 | 0.964 | 0.914 | 1 |
| OAS1 IFIT1 SIGLEC1 FAM20A RETN MMP8 | 0.880 | 0.870 | 0.889 | 0.907 | 0.841 | 0.974 | 0.950 | 0.886 | 1 |
| IFIT1 IFI44L SIGLEC1 FAM20A RETN MMP8 | 0.880 | 0.870 | 0.889 | 0.908 | 0.842 | 0.975 | 0.943 | 0.881 | 1 |
| SIGLEC1 ISG15 HERC6 IL1R2 FAM20A RETN | 0.880 | 0.870 | 0.889 | 0.910 | 0.848 | 0.972 | 0.959 | 0.898 | 1 |
| RSAD2 IFI27 IFI44L SIGLEC1 SLC1A2 FAM20A | 0.880 | 0.870 | 0.890 | 0.918 | 0.859 | 0.978 | 0.947 | 0.873 | 1 |
| OAS1 IFIT1 SIGLEC1 HERC6 SLC1A2 FAM20A | 0.880 | 0.871 | 0.889 | 0.916 | 0.862 | 0.971 | 0.937 | 0.860 | 1 |
| IFI27 IFI44L ISG15 HERC6 RETN MMP8 | 0.880 | 0.870 | 0.889 | 0.908 | 0.846 | 0.970 | 0.935 | 0.867 | 1 |
| RSAD2 IFI27 OAS1 SIGLEC1 ISG15 RETN | 0.880 | 0.871 | 0.889 | 0.909 | 0.847 | 0.971 | 0.955 | 0.896 | 1 |
| IFIT1 SIGLEC1 SLC1A2 IL1R2 FAM20A RETN | 0.880 | 0.871 | 0.889 | 0.918 | 0.865 | 0.971 | 0.955 | 0.898 | 1 |
| IFIT1 SIGLEC1 ISG15 SLC1A2 IL1R2 FAM20A | 0.880 | 0.871 | 0.889 | 0.917 | 0.863 | 0.971 | 0.951 | 0.891 | 1 |
| SIGLEC1 HERC6 IL1R2 OLAH FAM20A RETN | 0.880 | 0.870 | 0.889 | 0.910 | 0.846 | 0.973 | 0.960 | 0.901 | 1 |
| IFI27 OAS1 IFIT1 SLC1A2 IL1R2 MMP8 | 0.880 | 0.871 | 0.888 | 0.922 | 0.875 | 0.968 | 0.936 | 0.873 | 0.999 |
| IFI27 OAS1 IFIT1 ISG15 IL1R2 MMP8 | 0.880 | 0.871 | 0.888 | 0.916 | 0.866 | 0.966 | 0.943 | 0.891 | 0.996 |
| RSAD2 IFI27 IFIT1 SLC1A2 IL1R2 RETN | 0.880 | 0.871 | 0.888 | 0.919 | 0.867 | 0.971 | 0.938 | 0.876 | 1 |
| RSAD2 IFI27 OAS1 IFIT1 ISG15 IL1R2 | 0.880 | 0.871 | 0.888 | 0.914 | 0.863 | 0.965 | 0.947 | 0.896 | 0.998 |
| IFI27 IFIT1 IFI44L FAM20A RETN MMP8 | 0.880 | 0.869 | 0.890 | 0.915 | 0.852 | 0.977 | 0.946 | 0.870 | 1 |
| RSAD2 IFI27 OAS1 SLC1A2 IL1R2 RETN | 0.880 | 0.871 | 0.888 | 0.920 | 0.868 | 0.973 | 0.930 | 0.856 | 1 |
| IFI27 IFI44L SIGLEC1 ISG15 SLC1A2 FAM20A | 0.880 | 0.870 | 0.889 | 0.914 | 0.856 | 0.973 | 0.946 | 0.869 | 1 |
| OAS1 SIGLEC1 IL1R2 OLAH FAM20A RETN | 0.880 | 0.870 | 0.889 | 0.907 | 0.843 | 0.971 | 0.962 | 0.907 | 1 |
| RSAD2 IFIT1 SIGLEC1 ISG15 OLAH FAM20A | 0.880 | 0.870 | 0.889 | 0.904 | 0.834 | 0.974 | 0.953 | 0.902 | 1 |
| RSAD2 SIGLEC1 IL1R2 OLAH FAM20A RETN | 0.880 | 0.870 | 0.889 | 0.908 | 0.844 | 0.971 | 0.963 | 0.910 | 1 |
| IFI44L SLC1A2 IL1R2 OLAH FAM20A MMP8 | 0.880 | 0.871 | 0.888 | 0.917 | 0.866 | 0.969 | 0.928 | 0.847 | 1 |
| IFI27 OAS1 HERC6 SLC1A2 IL1R2 MMP8 | 0.880 | 0.871 | 0.888 | 0.922 | 0.876 | 0.967 | 0.924 | 0.855 | 0.993 |
| RSAD2 SIGLEC1 SLC1A2 IL1R2 FAM20A RETN | 0.880 | 0.870 | 0.889 | 0.915 | 0.862 | 0.969 | 0.954 | 0.888 | 1 |
| RSAD2 OAS1 SIGLEC1 OLAH FAM20A MMP8 | 0.880 | 0.870 | 0.889 | 0.905 | 0.839 | 0.971 | 0.958 | 0.910 | 1 |
| RSAD2 SIGLEC1 ISG15 IL1R2 OLAH FAM20A | 0.880 | 0.870 | 0.889 | 0.909 | 0.845 | 0.972 | 0.963 | 0.912 | 1 |
| SIGLEC1 ISG15 HERC6 SLC1A2 IL1R2 FAM20A | 0.879 | 0.870 | 0.889 | 0.919 | 0.866 | 0.972 | 0.949 | 0.876 | 1 |
| IFI27 IFI44L ISG15 SLC1A2 IL1R2 MMP8 | 0.879 | 0.872 | 0.887 | 0.922 | 0.875 | 0.968 | 0.930 | 0.866 | 0.995 |
| SIGLEC1 ISG15 SLC1A2 IL1R2 FAM20A RETN | 0.879 | 0.870 | 0.888 | 0.915 | 0.862 | 0.969 | 0.953 | 0.885 | 1 |
| RSAD2 IFI27 IFIT1 HERC6 SLC1A2 IL1R2 | 0.879 | 0.871 | 0.888 | 0.923 | 0.878 | 0.968 | 0.938 | 0.878 | 0.998 |
| IFI27 OAS1 ISG15 HERC6 SLC1A2 IL1R2 | 0.879 | 0.871 | 0.888 | 0.922 | 0.877 | 0.967 | 0.925 | 0.856 | 0.994 |
| RSAD2 IFI27 OAS1 IFIT1 SLC1A2 IL1R2 | 0.879 | 0.871 | 0.888 | 0.920 | 0.873 | 0.968 | 0.936 | 0.871 | 1 |
| RSAD2 IFI27 OAS1 IFI44L FAM20A RETN | 0.879 | 0.869 | 0.889 | 0.912 | 0.850 | 0.975 | 0.945 | 0.869 | 1 |
| IFI27 IFIT1 ISG15 SLC1A2 IL1R2 RETN | 0.879 | 0.871 | 0.888 | 0.920 | 0.869 | 0.971 | 0.937 | 0.874 | 1 |
| OAS1 SIGLEC1 HERC6 SLC1A2 OLAH FAM20A | 0.879 | 0.869 | 0.889 | 0.915 | 0.860 | 0.970 | 0.943 | 0.875 | 1 |
| RSAD2 IFI27 SLC1A2 IL1R2 RETN MMP8 | 0.879 | 0.871 | 0.888 | 0.923 | 0.873 | 0.973 | 0.929 | 0.859 | 1 |
| OAS1 IFI44L SIGLEC1 IL1R2 FAM20A RETN | 0.879 | 0.870 | 0.889 | 0.905 | 0.841 | 0.970 | 0.961 | 0.906 | 1 |
| IFI27 IFI44L SIGLEC1 HERC6 SLC1A2 RETN | 0.879 | 0.870 | 0.888 | 0.916 | 0.859 | 0.973 | 0.947 | 0.890 | 1 |
| IFI27 OAS1 IFIT1 IFI44L FAM20A RETN | 0.879 | 0.869 | 0.889 | 0.910 | 0.846 | 0.973 | 0.945 | 0.868 | 1 |
| RSAD2 SIGLEC1 HERC6 OLAH FAM20A MMP8 | 0.879 | 0.870 | 0.889 | 0.910 | 0.850 | 0.970 | 0.953 | 0.898 | 1 |
| IFIT1 IFI44L SIGLEC1 ISG15 FAM20A RETN | 0.879 | 0.870 | 0.888 | 0.900 | 0.830 | 0.970 | 0.945 | 0.886 | 1 |
| RSAD2 IFI27 IFI44L ISG15 FAM20A RETN | 0.879 | 0.869 | 0.889 | 0.910 | 0.846 | 0.974 | 0.942 | 0.865 | 1 |
| RSAD2 IFI27 OAS1 SIGLEC1 SLC1A2 RETN | 0.879 | 0.870 | 0.888 | 0.915 | 0.856 | 0.973 | 0.950 | 0.881 | 1 |
| RSAD2 IFI27 HERC6 SLC1A2 IL1R2 MMP8 | 0.879 | 0.871 | 0.887 | 0.921 | 0.875 | 0.967 | 0.930 | 0.866 | 0.995 |
| RSAD2 IFI27 IFIT1 SIGLEC1 ISG15 RETN | 0.879 | 0.870 | 0.888 | 0.908 | 0.847 | 0.970 | 0.955 | 0.900 | 1 |
| IFIT1 IFI44L SIGLEC1 ISG15 FAM20A MMP8 | 0.879 | 0.870 | 0.888 | 0.901 | 0.831 | 0.970 | 0.938 | 0.869 | 1 |
| IFI27 OAS1 SIGLEC1 ISG15 SLC1A2 RETN | 0.879 | 0.870 | 0.888 | 0.915 | 0.858 | 0.972 | 0.948 | 0.880 | 1 |
| IFI27 OAS1 IFI44L SIGLEC1 SLC1A2 FAM20A | 0.879 | 0.869 | 0.889 | 0.918 | 0.860 | 0.977 | 0.948 | 0.874 | 1 |
| IFI27 HERC6 SLC1A2 FAM20A RETN MMP8 | 0.879 | 0.869 | 0.889 | 0.919 | 0.861 | 0.977 | 0.939 | 0.859 | 1 |
| IFIT1 SIGLEC1 SLC1A2 OLAH FAM20A RETN | 0.879 | 0.870 | 0.888 | 0.919 | 0.864 | 0.974 | 0.943 | 0.882 | 1 |
| IFI44L SIGLEC1 ISG15 OLAH FAM20A MMP8 | 0.879 | 0.869 | 0.889 | 0.904 | 0.837 | 0.971 | 0.961 | 0.915 | 1 |
| IFI27 OAS1 IFI44L SIGLEC1 SLC1A2 RETN | 0.879 | 0.871 | 0.887 | 0.915 | 0.859 | 0.972 | 0.949 | 0.883 | 1 |
| RSAD2 IFI27 IFIT1 SLC1A2 IL1R2 MMP8 | 0.879 | 0.871 | 0.887 | 0.922 | 0.877 | 0.968 | 0.937 | 0.876 | 0.998 |
| RSAD2 IFI27 IFI44L HERC6 FAM20A MMP8 | 0.879 | 0.869 | 0.889 | 0.912 | 0.851 | 0.972 | 0.925 | 0.844 | 1 |
| RSAD2 IFI27 IFIT1 IFI44L SIGLEC1 RETN | 0.879 | 0.870 | 0.888 | 0.908 | 0.847 | 0.970 | 0.950 | 0.892 | 1 |
| RSAD2 SIGLEC1 HERC6 FAM20A RETN MMP8 | 0.879 | 0.869 | 0.888 | 0.909 | 0.846 | 0.973 | 0.948 | 0.882 | 1 |
| RSAD2 SIGLEC1 ISG15 SLC1A2 IL1R2 FAM20A | 0.879 | 0.869 | 0.888 | 0.914 | 0.859 | 0.969 | 0.951 | 0.882 | 1 |
| IFI27 OAS1 HERC6 SLC1A2 RETN MMP8 | 0.879 | 0.869 | 0.888 | 0.915 | 0.858 | 0.972 | 0.926 | 0.850 | 1 |
| RSAD2 IFI27 IFIT1 IFI44L FAM20A RETN | 0.879 | 0.869 | 0.889 | 0.910 | 0.847 | 0.974 | 0.942 | 0.865 | 1 |
| RSAD2 SIGLEC1 HERC6 SLC1A2 OLAH FAM20A | 0.879 | 0.868 | 0.889 | 0.916 | 0.862 | 0.971 | 0.940 | 0.869 | 1 |
| IFI27 ISG15 SLC1A2 IL1R2 RETN MMP8 | 0.879 | 0.870 | 0.887 | 0.922 | 0.871 | 0.972 | 0.923 | 0.845 | 1 |
| IFI44L SIGLEC1 SLC1A2 OLAH FAM20A RETN | 0.879 | 0.869 | 0.888 | 0.914 | 0.855 | 0.972 | 0.948 | 0.884 | 1 |
| IFI27 IFIT1 ISG15 HERC6 FAM20A MMP8 | 0.879 | 0.869 | 0.889 | 0.909 | 0.849 | 0.969 | 0.924 | 0.839 | 1 |
| RSAD2 IFI44L SIGLEC1 ISG15 IL1R2 FAM20A | 0.879 | 0.869 | 0.888 | 0.910 | 0.846 | 0.973 | 0.963 | 0.912 | 1 |
| RSAD2 IFI27 OAS1 SIGLEC1 RETN MMP8 | 0.879 | 0.869 | 0.889 | 0.912 | 0.851 | 0.973 | 0.958 | 0.897 | 1 |
| IFI27 OAS1 IFI44L SLC1A2 IL1R2 RETN | 0.879 | 0.870 | 0.887 | 0.924 | 0.875 | 0.972 | 0.932 | 0.865 | 0.998 |
| SIGLEC1 HERC6 OLAH FAM20A RETN MMP8 | 0.878 | 0.868 | 0.889 | 0.909 | 0.842 | 0.975 | 0.963 | 0.910 | 1 |
| IFI27 OAS1 IFIT1 ISG15 SLC1A2 IL1R2 | 0.878 | 0.870 | 0.887 | 0.921 | 0.875 | 0.968 | 0.936 | 0.874 | 0.998 |
| IFI27 OAS1 IFI44L ISG15 FAM20A RETN | 0.878 | 0.869 | 0.888 | 0.911 | 0.848 | 0.974 | 0.946 | 0.869 | 1 |
| RSAD2 IFI27 IFI44L ISG15 IL1R2 MMP8 | 0.878 | 0.871 | 0.886 | 0.914 | 0.862 | 0.966 | 0.936 | 0.880 | 0.992 |
| OAS1 IFIT1 IFI44L SIGLEC1 FAM20A RETN | 0.878 | 0.869 | 0.888 | 0.902 | 0.832 | 0.972 | 0.943 | 0.882 | 1 |
| IFIT1 IFI44L SIGLEC1 ISG15 SLC1A2 FAM20A | 0.878 | 0.869 | 0.887 | 0.911 | 0.851 | 0.970 | 0.936 | 0.861 | 1 |
| IFI27 OAS1 SIGLEC1 ISG15 RETN MMP8 | 0.878 | 0.869 | 0.888 | 0.912 | 0.851 | 0.973 | 0.957 | 0.897 | 1 |
| IFI44L SIGLEC1 HERC6 OLAH FAM20A MMP8 | 0.878 | 0.868 | 0.888 | 0.905 | 0.840 | 0.970 | 0.961 | 0.906 | 1 |
| RSAD2 IFI27 IFI44L SLC1A2 IL1R2 RETN | 0.878 | 0.870 | 0.886 | 0.922 | 0.871 | 0.973 | 0.929 | 0.860 | 0.998 |
| OAS1 SIGLEC1 ISG15 IL1R2 FAM20A RETN | 0.878 | 0.869 | 0.888 | 0.906 | 0.842 | 0.971 | 0.961 | 0.904 | 1 |
| RSAD2 IFI27 OAS1 IFI44L ISG15 IL1R2 | 0.878 | 0.870 | 0.886 | 0.914 | 0.863 | 0.966 | 0.940 | 0.886 | 0.994 |
| IFI27 IFI44L SIGLEC1 HERC6 SLC1A2 FAM20A | 0.878 | 0.869 | 0.888 | 0.914 | 0.856 | 0.972 | 0.947 | 0.870 | 1 |
| IFI44L SIGLEC1 HERC6 SLC1A2 OLAH FAM20A | 0.878 | 0.868 | 0.888 | 0.914 | 0.857 | 0.972 | 0.946 | 0.872 | 1 |
| RSAD2 SIGLEC1 ISG15 IL1R2 FAM20A RETN | 0.878 | 0.869 | 0.888 | 0.906 | 0.842 | 0.971 | 0.962 | 0.907 | 1 |
| IFI27 IFIT1 HERC6 SLC1A2 IL1R2 MMP8 | 0.878 | 0.870 | 0.887 | 0.921 | 0.875 | 0.967 | 0.937 | 0.877 | 0.997 |
| IFI27 SIGLEC1 ISG15 SLC1A2 FAM20A MMP8 | 0.878 | 0.868 | 0.888 | 0.912 | 0.851 | 0.973 | 0.945 | 0.868 | 1 |
| IFIT1 IFI44L SIGLEC1 SLC1A2 FAM20A MMP8 | 0.878 | 0.869 | 0.887 | 0.911 | 0.852 | 0.970 | 0.938 | 0.864 | 1 |
| IFI44L SIGLEC1 OLAH FAM20A RETN MMP8 | 0.878 | 0.868 | 0.888 | 0.905 | 0.838 | 0.972 | 0.966 | 0.923 | 1 |
| IFI27 IFIT1 ISG15 SLC1A2 IL1R2 MMP8 | 0.878 | 0.870 | 0.886 | 0.922 | 0.877 | 0.968 | 0.937 | 0.877 | 0.997 |
| OAS1 SIGLEC1 SLC1A2 IL1R2 FAM20A RETN | 0.878 | 0.869 | 0.887 | 0.915 | 0.861 | 0.969 | 0.955 | 0.891 | 1 |
| RSAD2 IFIT1 SIGLEC1 OLAH FAM20A RETN | 0.878 | 0.869 | 0.888 | 0.903 | 0.834 | 0.973 | 0.953 | 0.902 | 1 |
| OAS1 IFIT1 SIGLEC1 ISG15 FAM20A RETN | 0.878 | 0.869 | 0.888 | 0.903 | 0.833 | 0.972 | 0.949 | 0.889 | 1 |
| OAS1 IFIT1 IFI44L SIGLEC1 ISG15 FAM20A | 0.878 | 0.869 | 0.888 | 0.902 | 0.832 | 0.972 | 0.938 | 0.870 | 1 |
| RSAD2 IFI27 OAS1 IFI44L HERC6 FAM20A | 0.878 | 0.868 | 0.888 | 0.910 | 0.848 | 0.972 | 0.930 | 0.851 | 1 |
| IFI27 OAS1 SLC1A2 IL1R2 RETN MMP8 | 0.878 | 0.869 | 0.887 | 0.924 | 0.875 | 0.973 | 0.932 | 0.864 | 0.999 |
| IFI27 OAS1 SIGLEC1 SLC1A2 RETN MMP8 | 0.878 | 0.869 | 0.887 | 0.915 | 0.857 | 0.972 | 0.948 | 0.880 | 1 |
| RSAD2 OAS1 IFIT1 IFI44L SIGLEC1 FAM20A | 0.878 | 0.869 | 0.887 | 0.902 | 0.832 | 0.973 | 0.939 | 0.873 | 1 |
| RSAD2 IFI27 OAS1 ISG15 IL1R2 MMP8 | 0.878 | 0.870 | 0.886 | 0.916 | 0.865 | 0.966 | 0.939 | 0.884 | 0.994 |
| SIGLEC1 ISG15 HERC6 OLAH FAM20A MMP8 | 0.878 | 0.868 | 0.888 | 0.903 | 0.835 | 0.970 | 0.964 | 0.916 | 1 |
| RSAD2 IFI27 ISG15 SLC1A2 IL1R2 RETN | 0.878 | 0.870 | 0.886 | 0.922 | 0.871 | 0.973 | 0.930 | 0.862 | 0.999 |
| RSAD2 IFI27 IFI44L FAM20A RETN MMP8 | 0.878 | 0.867 | 0.888 | 0.915 | 0.852 | 0.977 | 0.945 | 0.869 | 1 |
| IFI27 IFIT1 IFI44L SLC1A2 IL1R2 MMP8 | 0.878 | 0.870 | 0.886 | 0.921 | 0.874 | 0.968 | 0.939 | 0.880 | 0.998 |
| IFI27 OAS1 IFIT1 ISG15 FAM20A RETN | 0.878 | 0.868 | 0.887 | 0.909 | 0.847 | 0.972 | 0.943 | 0.865 | 1 |
| IFI27 IFI44L HERC6 SLC1A2 IL1R2 MMP8 | 0.878 | 0.869 | 0.887 | 0.920 | 0.874 | 0.966 | 0.927 | 0.861 | 0.993 |
| RSAD2 OAS1 SIGLEC1 SLC1A2 IL1R2 FAM20A | 0.878 | 0.868 | 0.887 | 0.915 | 0.860 | 0.970 | 0.955 | 0.894 | 1 |
| IFI27 OAS1 IFI44L ISG15 IL1R2 MMP8 | 0.878 | 0.870 | 0.886 | 0.914 | 0.863 | 0.965 | 0.932 | 0.873 | 0.990 |
| IFI27 OAS1 IFIT1 SIGLEC1 HERC6 MMP8 | 0.878 | 0.869 | 0.886 | 0.907 | 0.852 | 0.962 | 0.904 | 0.828 | 0.981 |
| SIGLEC1 ISG15 HERC6 SLC1A2 OLAH FAM20A | 0.878 | 0.868 | 0.888 | 0.914 | 0.857 | 0.970 | 0.946 | 0.872 | 1 |
| RSAD2 IFI27 ISG15 HERC6 SLC1A2 IL1R2 | 0.878 | 0.870 | 0.886 | 0.921 | 0.876 | 0.967 | 0.928 | 0.861 | 0.995 |
| RSAD2 IFI44L SIGLEC1 SLC1A2 IL1R2 FAM20A | 0.878 | 0.869 | 0.887 | 0.916 | 0.862 | 0.971 | 0.954 | 0.890 | 1 |
| IFI44L ISG15 HERC6 IL1R2 OLAH FAM20A | 0.878 | 0.868 | 0.887 | 0.905 | 0.844 | 0.967 | 0.939 | 0.867 | 1 |
| IFI27 IFI44L SIGLEC1 SLC1A2 FAM20A MMP8 | 0.878 | 0.868 | 0.887 | 0.911 | 0.849 | 0.972 | 0.946 | 0.869 | 1 |
| SIGLEC1 HERC6 SLC1A2 IL1R2 FAM20A RETN | 0.878 | 0.868 | 0.887 | 0.918 | 0.865 | 0.971 | 0.948 | 0.875 | 1 |
| RSAD2 IFI27 IFI44L HERC6 SLC1A2 IL1R2 | 0.878 | 0.869 | 0.886 | 0.919 | 0.873 | 0.966 | 0.928 | 0.861 | 0.995 |
| OAS1 IFIT1 IFI44L SIGLEC1 FAM20A MMP8 | 0.878 | 0.868 | 0.887 | 0.902 | 0.832 | 0.972 | 0.939 | 0.870 | 1 |
| OAS1 IFI44L SIGLEC1 SLC1A2 OLAH FAM20A | 0.878 | 0.868 | 0.887 | 0.913 | 0.854 | 0.971 | 0.948 | 0.890 | 1 |
| RSAD2 OAS1 SIGLEC1 SLC1A2 OLAH FAM20A | 0.878 | 0.868 | 0.887 | 0.915 | 0.857 | 0.972 | 0.950 | 0.892 | 1 |
| IFI27 OAS1 IFI44L HERC6 SLC1A2 IL1R2 | 0.878 | 0.869 | 0.886 | 0.921 | 0.876 | 0.967 | 0.926 | 0.859 | 0.993 |
| RSAD2 IFI27 IFIT1 ISG15 SLC1A2 IL1R2 | 0.878 | 0.870 | 0.886 | 0.922 | 0.876 | 0.968 | 0.936 | 0.875 | 0.997 |
| OAS1 IFI44L SIGLEC1 SLC1A2 IL1R2 FAM20A | 0.878 | 0.868 | 0.887 | 0.916 | 0.861 | 0.970 | 0.954 | 0.891 | 1 |
| IFI27 IFIT1 IFI44L ISG15 SLC1A2 IL1R2 | 0.877 | 0.869 | 0.886 | 0.923 | 0.876 | 0.969 | 0.934 | 0.873 | 0.994 |
| IFI27 IFIT1 IFI44L SIGLEC1 RETN MMP8 | 0.877 | 0.868 | 0.887 | 0.909 | 0.848 | 0.971 | 0.953 | 0.897 | 1 |
| RSAD2 IFI44L SIGLEC1 IL1R2 FAM20A RETN | 0.877 | 0.868 | 0.887 | 0.906 | 0.842 | 0.970 | 0.961 | 0.906 | 1 |
| IFI44L SIGLEC1 ISG15 HERC6 OLAH FAM20A | 0.877 | 0.868 | 0.887 | 0.903 | 0.836 | 0.970 | 0.953 | 0.894 | 1 |
| IFI27 IFIT1 ISG15 FAM20A RETN MMP8 | 0.877 | 0.867 | 0.888 | 0.912 | 0.850 | 0.975 | 0.943 | 0.867 | 1 |
| RSAD2 IFI27 OAS1 SLC1A2 IL1R2 MMP8 | 0.877 | 0.869 | 0.886 | 0.920 | 0.872 | 0.967 | 0.930 | 0.863 | 0.998 |
| IFI27 OAS1 ISG15 SLC1A2 IL1R2 RETN | 0.877 | 0.869 | 0.886 | 0.922 | 0.871 | 0.972 | 0.926 | 0.856 | 0.997 |
| IFI27 IFIT1 SIGLEC1 ISG15 RETN MMP8 | 0.877 | 0.868 | 0.887 | 0.908 | 0.846 | 0.970 | 0.957 | 0.902 | 1 |
| IFI27 SIGLEC1 ISG15 HERC6 SLC1A2 FAM20A | 0.877 | 0.867 | 0.887 | 0.911 | 0.851 | 0.972 | 0.947 | 0.870 | 1 |
| OAS1 SIGLEC1 ISG15 SLC1A2 OLAH FAM20A | 0.877 | 0.868 | 0.887 | 0.912 | 0.854 | 0.971 | 0.950 | 0.891 | 1 |
| IFI27 OAS1 IFI44L SIGLEC1 RETN MMP8 | 0.877 | 0.868 | 0.887 | 0.911 | 0.850 | 0.972 | 0.953 | 0.893 | 1 |
| OAS1 SIGLEC1 ISG15 SLC1A2 IL1R2 FAM20A | 0.877 | 0.868 | 0.887 | 0.914 | 0.859 | 0.969 | 0.953 | 0.888 | 1 |
| IFI27 IFIT1 ISG15 HERC6 SLC1A2 IL1R2 | 0.877 | 0.869 | 0.886 | 0.923 | 0.877 | 0.968 | 0.937 | 0.877 | 0.997 |
| IFI44L ISG15 SLC1A2 OLAH FAM20A MMP8 | 0.877 | 0.868 | 0.887 | 0.911 | 0.854 | 0.969 | 0.925 | 0.850 | 1 |
| RSAD2 IFI27 IFIT1 IFI44L HERC6 FAM20A | 0.877 | 0.867 | 0.887 | 0.907 | 0.844 | 0.970 | 0.925 | 0.844 | 1 |
| IFI27 OAS1 IFIT1 IFI44L SLC1A2 IL1R2 | 0.877 | 0.869 | 0.886 | 0.921 | 0.874 | 0.968 | 0.937 | 0.877 | 0.997 |
| RSAD2 IFI27 IFIT1 HERC6 FAM20A MMP8 | 0.877 | 0.868 | 0.887 | 0.911 | 0.852 | 0.969 | 0.926 | 0.842 | 1 |
| RSAD2 IFI27 ISG15 HERC6 FAM20A MMP8 | 0.877 | 0.867 | 0.887 | 0.912 | 0.854 | 0.970 | 0.926 | 0.845 | 1 |
| RSAD2 IFI27 OAS1 ISG15 FAM20A RETN | 0.877 | 0.867 | 0.887 | 0.910 | 0.849 | 0.971 | 0.940 | 0.862 | 1 |
| IFI27 OAS1 ISG15 HERC6 FAM20A MMP8 | 0.877 | 0.867 | 0.887 | 0.913 | 0.856 | 0.970 | 0.927 | 0.846 | 1 |
| IFIT1 IFI44L SLC1A2 OLAH FAM20A MMP8 | 0.877 | 0.868 | 0.886 | 0.913 | 0.856 | 0.970 | 0.924 | 0.848 | 1.000 |
| SIGLEC1 ISG15 OLAH FAM20A RETN MMP8 | 0.877 | 0.867 | 0.887 | 0.904 | 0.837 | 0.972 | 0.970 | 0.929 | 1 |
| IFI27 OAS1 IFIT1 HERC6 FAM20A MMP8 | 0.877 | 0.867 | 0.887 | 0.911 | 0.853 | 0.970 | 0.925 | 0.842 | 1 |
| IFI27 OAS1 IFI44L HERC6 FAM20A MMP8 | 0.877 | 0.867 | 0.887 | 0.910 | 0.850 | 0.971 | 0.926 | 0.845 | 1 |
| RSAD2 IFI27 ISG15 SLC1A2 IL1R2 MMP8 | 0.877 | 0.869 | 0.885 | 0.921 | 0.874 | 0.968 | 0.930 | 0.866 | 0.995 |
| RSAD2 OAS1 IFIT1 SIGLEC1 FAM20A RETN | 0.877 | 0.867 | 0.887 | 0.903 | 0.834 | 0.972 | 0.949 | 0.889 | 1 |
| OAS1 IFIT1 IFI44L SIGLEC1 SLC1A2 FAM20A | 0.877 | 0.868 | 0.886 | 0.912 | 0.853 | 0.971 | 0.936 | 0.861 | 1 |
| RSAD2 IFI44L SIGLEC1 SLC1A2 OLAH FAM20A | 0.877 | 0.867 | 0.887 | 0.915 | 0.857 | 0.973 | 0.945 | 0.885 | 1 |
| RSAD2 IFI27 IFI44L SIGLEC1 ISG15 RETN | 0.877 | 0.868 | 0.886 | 0.908 | 0.846 | 0.969 | 0.950 | 0.892 | 1 |
| RSAD2 OAS1 SIGLEC1 HERC6 OLAH FAM20A | 0.877 | 0.867 | 0.887 | 0.906 | 0.844 | 0.969 | 0.948 | 0.891 | 1 |
| OAS1 IFIT1 SIGLEC1 ISG15 FAM20A MMP8 | 0.877 | 0.867 | 0.886 | 0.901 | 0.832 | 0.971 | 0.946 | 0.877 | 1 |
| RSAD2 SIGLEC1 ISG15 HERC6 FAM20A RETN | 0.877 | 0.867 | 0.886 | 0.910 | 0.849 | 0.971 | 0.947 | 0.881 | 1 |
| OAS1 SIGLEC1 HERC6 FAM20A RETN MMP8 | 0.877 | 0.867 | 0.886 | 0.908 | 0.842 | 0.973 | 0.952 | 0.889 | 1 |
| IFI27 OAS1 IFI44L FAM20A RETN MMP8 | 0.877 | 0.866 | 0.887 | 0.914 | 0.851 | 0.976 | 0.947 | 0.871 | 1 |
| IFI27 IFIT1 IFI44L HERC6 SLC1A2 IL1R2 | 0.877 | 0.868 | 0.885 | 0.923 | 0.877 | 0.969 | 0.936 | 0.876 | 0.995 |
| RSAD2 IFI27 OAS1 IFI44L SLC1A2 IL1R2 | 0.877 | 0.868 | 0.885 | 0.921 | 0.874 | 0.967 | 0.933 | 0.866 | 0.999 |
| RSAD2 OAS1 IFIT1 SIGLEC1 ISG15 FAM20A | 0.877 | 0.867 | 0.886 | 0.900 | 0.829 | 0.970 | 0.946 | 0.879 | 1 |
| IFI44L ISG15 SLC1A2 IL1R2 OLAH FAM20A | 0.877 | 0.868 | 0.886 | 0.913 | 0.857 | 0.969 | 0.928 | 0.852 | 1 |
| IFI27 SIGLEC1 HERC6 SLC1A2 FAM20A MMP8 | 0.877 | 0.867 | 0.887 | 0.912 | 0.851 | 0.973 | 0.946 | 0.869 | 1 |
| RSAD2 IFI27 IFIT1 IFI44L SLC1A2 IL1R2 | 0.877 | 0.869 | 0.885 | 0.921 | 0.874 | 0.968 | 0.938 | 0.879 | 0.997 |
| RSAD2 IFI27 OAS1 IFIT1 FAM20A RETN | 0.877 | 0.867 | 0.886 | 0.911 | 0.849 | 0.972 | 0.943 | 0.867 | 1 |
| SIGLEC1 ISG15 SLC1A2 OLAH FAM20A RETN | 0.877 | 0.867 | 0.886 | 0.914 | 0.856 | 0.971 | 0.949 | 0.887 | 1 |
| RSAD2 OAS1 IFIT1 SIGLEC1 FAM20A MMP8 | 0.877 | 0.867 | 0.886 | 0.901 | 0.832 | 0.971 | 0.949 | 0.883 | 1 |
| IFIT1 SIGLEC1 ISG15 FAM20A RETN MMP8 | 0.877 | 0.867 | 0.886 | 0.906 | 0.840 | 0.972 | 0.947 | 0.885 | 1 |
| RSAD2 SIGLEC1 ISG15 SLC1A2 OLAH FAM20A | 0.877 | 0.867 | 0.886 | 0.912 | 0.854 | 0.971 | 0.950 | 0.891 | 1 |
| IFI44L HERC6 IL1R2 OLAH FAM20A MMP8 | 0.877 | 0.867 | 0.886 | 0.909 | 0.850 | 0.968 | 0.940 | 0.865 | 1 |
| IFI27 IFI44L ISG15 SLC1A2 FAM20A RETN | 0.877 | 0.867 | 0.886 | 0.911 | 0.850 | 0.973 | 0.941 | 0.861 | 1 |
| IFI27 SIGLEC1 ISG15 HERC6 RETN MMP8 | 0.877 | 0.866 | 0.887 | 0.905 | 0.840 | 0.970 | 0.960 | 0.910 | 1 |
| RSAD2 IFI27 IFIT1 ISG15 FAM20A RETN | 0.877 | 0.867 | 0.886 | 0.911 | 0.849 | 0.972 | 0.942 | 0.865 | 1 |
| IFIT1 SIGLEC1 ISG15 OLAH FAM20A RETN | 0.876 | 0.867 | 0.886 | 0.903 | 0.833 | 0.972 | 0.948 | 0.893 | 1 |
| IFI27 IFI44L ISG15 HERC6 SLC1A2 IL1R2 | 0.876 | 0.868 | 0.885 | 0.921 | 0.876 | 0.967 | 0.929 | 0.864 | 0.995 |
| IFI27 IFI44L SIGLEC1 ISG15 RETN MMP8 | 0.876 | 0.867 | 0.886 | 0.906 | 0.843 | 0.969 | 0.951 | 0.894 | 1 |
| RSAD2 SIGLEC1 ISG15 HERC6 OLAH FAM20A | 0.876 | 0.867 | 0.886 | 0.909 | 0.847 | 0.971 | 0.949 | 0.892 | 1 |
| RSAD2 IFIT1 IFI44L SIGLEC1 ISG15 FAM20A | 0.876 | 0.867 | 0.885 | 0.899 | 0.828 | 0.969 | 0.940 | 0.877 | 1 |
| IFI27 IFIT1 SIGLEC1 ISG15 SLC1A2 RETN | 0.876 | 0.868 | 0.885 | 0.915 | 0.860 | 0.971 | 0.954 | 0.897 | 1 |
| RSAD2 IFI27 IFI44L SLC1A2 IL1R2 MMP8 | 0.876 | 0.868 | 0.884 | 0.921 | 0.873 | 0.968 | 0.933 | 0.868 | 0.997 |
| RSAD2 IFI27 OAS1 HERC6 FAM20A MMP8 | 0.876 | 0.866 | 0.886 | 0.912 | 0.853 | 0.970 | 0.927 | 0.846 | 1 |
| IFI27 ISG15 HERC6 SLC1A2 IL1R2 MMP8 | 0.876 | 0.868 | 0.885 | 0.922 | 0.876 | 0.967 | 0.928 | 0.863 | 0.994 |
| OAS1 SIGLEC1 SLC1A2 OLAH FAM20A RETN | 0.876 | 0.866 | 0.886 | 0.913 | 0.855 | 0.972 | 0.952 | 0.896 | 1 |
| ISG15 HERC6 IL1R2 OLAH FAM20A MMP8 | 0.876 | 0.867 | 0.886 | 0.908 | 0.850 | 0.966 | 0.935 | 0.855 | 1 |
| IFI44L ISG15 HERC6 IL1R2 FAM20A MMP8 | 0.876 | 0.867 | 0.885 | 0.910 | 0.850 | 0.969 | 0.932 | 0.847 | 1 |
| RSAD2 SIGLEC1 ISG15 OLAH FAM20A MMP8 | 0.876 | 0.866 | 0.886 | 0.904 | 0.838 | 0.971 | 0.962 | 0.920 | 1 |
| RSAD2 OAS1 ISG15 HERC6 IL1R2 FAM20A | 0.876 | 0.867 | 0.886 | 0.904 | 0.844 | 0.965 | 0.933 | 0.850 | 1 |
| IFIT1 IFI44L HERC6 SLC1A2 OLAH FAM20A | 0.876 | 0.866 | 0.886 | 0.916 | 0.862 | 0.969 | 0.916 | 0.830 | 1 |
| RSAD2 IFIT1 IFI44L SIGLEC1 SLC1A2 FAM20A | 0.876 | 0.867 | 0.885 | 0.910 | 0.851 | 0.970 | 0.936 | 0.861 | 1 |
| RSAD2 IFI27 OAS1 ISG15 SLC1A2 IL1R2 | 0.876 | 0.868 | 0.884 | 0.922 | 0.876 | 0.968 | 0.929 | 0.861 | 0.998 |
| RSAD2 SIGLEC1 SLC1A2 OLAH FAM20A RETN | 0.876 | 0.866 | 0.886 | 0.914 | 0.856 | 0.972 | 0.951 | 0.894 | 1 |
| IFIT1 IFI44L SIGLEC1 SLC1A2 OLAH MMP8 | 0.876 | 0.867 | 0.885 | 0.911 | 0.855 | 0.967 | 0.953 | 0.909 | 0.998 |
| IFI27 IFI44L SLC1A2 FAM20A RETN MMP8 | 0.876 | 0.866 | 0.886 | 0.912 | 0.851 | 0.974 | 0.941 | 0.862 | 1 |
| OAS1 HERC6 IL1R2 OLAH FAM20A MMP8 | 0.876 | 0.866 | 0.885 | 0.909 | 0.849 | 0.968 | 0.937 | 0.859 | 1 |
| OAS1 SIGLEC1 ISG15 HERC6 FAM20A RETN | 0.876 | 0.866 | 0.886 | 0.907 | 0.845 | 0.969 | 0.952 | 0.889 | 1 |
| OAS1 IFIT1 ISG15 HERC6 IL1R2 FAM20A | 0.876 | 0.866 | 0.885 | 0.904 | 0.843 | 0.965 | 0.933 | 0.851 | 1 |
| RSAD2 IFI27 ISG15 HERC6 SLC1A2 RETN | 0.876 | 0.866 | 0.885 | 0.914 | 0.857 | 0.972 | 0.937 | 0.870 | 1 |
| RSAD2 IFI27 IFI44L HERC6 SLC1A2 RETN | 0.876 | 0.866 | 0.885 | 0.914 | 0.855 | 0.972 | 0.936 | 0.868 | 1 |
| OAS1 SIGLEC1 HERC6 OLAH FAM20A MMP8 | 0.876 | 0.866 | 0.886 | 0.906 | 0.843 | 0.969 | 0.958 | 0.907 | 1 |
| IFIT1 SIGLEC1 HERC6 IL1R2 RETN MMP8 | 0.876 | 0.866 | 0.885 | 0.904 | 0.841 | 0.966 | 0.963 | 0.925 | 1 |
| RSAD2 IFI27 IFIT1 SIGLEC1 SLC1A2 RETN | 0.876 | 0.867 | 0.884 | 0.915 | 0.858 | 0.971 | 0.953 | 0.896 | 1 |
| IFIT1 IFI44L ISG15 HERC6 IL1R2 FAM20A | 0.876 | 0.867 | 0.885 | 0.908 | 0.848 | 0.968 | 0.932 | 0.851 | 1 |
| OAS1 IFIT1 SIGLEC1 ISG15 SLC1A2 FAM20A | 0.876 | 0.866 | 0.885 | 0.907 | 0.846 | 0.968 | 0.943 | 0.871 | 1 |
| OAS1 IFI44L SIGLEC1 OLAH FAM20A MMP8 | 0.876 | 0.865 | 0.886 | 0.901 | 0.834 | 0.969 | 0.963 | 0.922 | 1 |
| OAS1 IFIT1 HERC6 IL1R2 FAM20A MMP8 | 0.876 | 0.866 | 0.885 | 0.905 | 0.844 | 0.967 | 0.939 | 0.860 | 1 |
| SIGLEC1 ISG15 HERC6 FAM20A RETN MMP8 | 0.875 | 0.865 | 0.886 | 0.904 | 0.835 | 0.972 | 0.957 | 0.891 | 1 |
| RSAD2 IFI27 IFI44L ISG15 SLC1A2 IL1R2 | 0.875 | 0.868 | 0.883 | 0.922 | 0.876 | 0.968 | 0.930 | 0.865 | 0.996 |
| RSAD2 OAS1 HERC6 IL1R2 FAM20A MMP8 | 0.875 | 0.866 | 0.885 | 0.904 | 0.843 | 0.966 | 0.934 | 0.853 | 1 |
| IFI44L SIGLEC1 SLC1A2 IL1R2 OLAH MMP8 | 0.875 | 0.867 | 0.884 | 0.912 | 0.858 | 0.967 | 0.966 | 0.931 | 1 |
| RSAD2 OAS1 HERC6 IL1R2 OLAH FAM20A | 0.875 | 0.866 | 0.885 | 0.906 | 0.845 | 0.968 | 0.938 | 0.864 | 1 |
| IFI27 IFI44L SIGLEC1 ISG15 SLC1A2 RETN | 0.875 | 0.867 | 0.884 | 0.914 | 0.857 | 0.971 | 0.951 | 0.893 | 1 |
| IFI27 IFIT1 IFI44L SIGLEC1 SLC1A2 RETN | 0.875 | 0.867 | 0.884 | 0.916 | 0.860 | 0.972 | 0.952 | 0.894 | 1 |
| IFI44L SIGLEC1 HERC6 FAM20A RETN MMP8 | 0.875 | 0.865 | 0.885 | 0.907 | 0.840 | 0.974 | 0.955 | 0.888 | 1 |
| RSAD2 IFI27 SIGLEC1 ISG15 RETN MMP8 | 0.875 | 0.866 | 0.885 | 0.912 | 0.852 | 0.973 | 0.957 | 0.902 | 1 |
| IFI44L ISG15 HERC6 IL1R2 FAM20A RETN | 0.875 | 0.866 | 0.885 | 0.908 | 0.848 | 0.969 | 0.942 | 0.868 | 1 |
| RSAD2 IFI27 IFIT1 SIGLEC1 RETN MMP8 | 0.875 | 0.866 | 0.885 | 0.910 | 0.849 | 0.971 | 0.955 | 0.900 | 1 |
| IFIT1 IFI44L SIGLEC1 SLC1A2 FAM20A RETN | 0.875 | 0.866 | 0.885 | 0.913 | 0.854 | 0.972 | 0.936 | 0.863 | 1 |
| IFIT1 ISG15 HERC6 IL1R2 OLAH FAM20A | 0.875 | 0.866 | 0.885 | 0.906 | 0.846 | 0.966 | 0.935 | 0.858 | 1 |
| IFI44L SIGLEC1 ISG15 SLC1A2 OLAH MMP8 | 0.875 | 0.867 | 0.884 | 0.912 | 0.857 | 0.968 | 0.952 | 0.907 | 0.997 |
| IFIT1 IFI44L HERC6 IL1R2 OLAH FAM20A | 0.875 | 0.866 | 0.884 | 0.906 | 0.845 | 0.966 | 0.934 | 0.856 | 1 |
| IFI27 IFIT1 IFI44L SLC1A2 FAM20A RETN | 0.875 | 0.865 | 0.885 | 0.911 | 0.850 | 0.973 | 0.937 | 0.855 | 1 |
| RSAD2 IFI44L SIGLEC1 HERC6 OLAH FAM20A | 0.875 | 0.866 | 0.885 | 0.907 | 0.844 | 0.970 | 0.946 | 0.888 | 1 |
| RSAD2 IFIT1 SIGLEC1 FAM20A RETN MMP8 | 0.875 | 0.866 | 0.885 | 0.904 | 0.837 | 0.972 | 0.948 | 0.886 | 1 |
| RSAD2 IFIT1 IFI44L SIGLEC1 FAM20A RETN | 0.875 | 0.866 | 0.885 | 0.901 | 0.831 | 0.971 | 0.945 | 0.885 | 1 |
| IFIT1 HERC6 IL1R2 OLAH FAM20A MMP8 | 0.875 | 0.866 | 0.885 | 0.907 | 0.847 | 0.966 | 0.946 | 0.875 | 1 |
| RSAD2 IFI27 IFIT1 HERC6 SLC1A2 RETN | 0.875 | 0.866 | 0.885 | 0.912 | 0.854 | 0.971 | 0.938 | 0.871 | 1 |
| RSAD2 OAS1 IL1R2 OLAH FAM20A MMP8 | 0.875 | 0.866 | 0.884 | 0.906 | 0.844 | 0.967 | 0.935 | 0.856 | 1 |
| RSAD2 ISG15 HERC6 IL1R2 OLAH FAM20A | 0.875 | 0.865 | 0.885 | 0.905 | 0.846 | 0.965 | 0.934 | 0.857 | 1 |
| RSAD2 IFI44L ISG15 HERC6 IL1R2 FAM20A | 0.875 | 0.866 | 0.884 | 0.909 | 0.851 | 0.967 | 0.934 | 0.855 | 1 |
| IFI27 OAS1 IFIT1 FAM20A RETN MMP8 | 0.875 | 0.865 | 0.885 | 0.911 | 0.849 | 0.974 | 0.945 | 0.869 | 1 |
| IFI27 OAS1 IFI44L SLC1A2 IL1R2 MMP8 | 0.875 | 0.867 | 0.883 | 0.922 | 0.876 | 0.968 | 0.927 | 0.860 | 0.994 |
| RSAD2 OAS1 IFIT1 SIGLEC1 SLC1A2 FAM20A | 0.875 | 0.865 | 0.885 | 0.907 | 0.846 | 0.968 | 0.943 | 0.871 | 1 |
| RSAD2 IFI44L SIGLEC1 OLAH FAM20A MMP8 | 0.875 | 0.865 | 0.885 | 0.904 | 0.838 | 0.971 | 0.964 | 0.924 | 1 |
| ISG15 HERC6 IL1R2 FAM20A RETN MMP8 | 0.875 | 0.866 | 0.885 | 0.910 | 0.850 | 0.969 | 0.932 | 0.848 | 1 |
| RSAD2 IFI27 OAS1 FAM20A RETN MMP8 | 0.875 | 0.865 | 0.885 | 0.908 | 0.845 | 0.970 | 0.942 | 0.865 | 1 |
| RSAD2 SIGLEC1 HERC6 OLAH FAM20A RETN | 0.875 | 0.865 | 0.885 | 0.907 | 0.844 | 0.969 | 0.947 | 0.889 | 1 |
| IFI27 OAS1 SIGLEC1 HERC6 SLC1A2 MMP8 | 0.875 | 0.866 | 0.884 | 0.911 | 0.857 | 0.965 | 0.925 | 0.856 | 0.994 |
| IFI27 IFIT1 IFI44L SIGLEC1 HERC6 MMP8 | 0.875 | 0.866 | 0.884 | 0.905 | 0.847 | 0.963 | 0.908 | 0.833 | 0.982 |
| RSAD2 IFIT1 IFI44L SIGLEC1 FAM20A MMP8 | 0.875 | 0.865 | 0.885 | 0.901 | 0.832 | 0.971 | 0.938 | 0.869 | 1 |
| OAS1 IFIT1 SIGLEC1 SLC1A2 FAM20A MMP8 | 0.875 | 0.865 | 0.884 | 0.908 | 0.847 | 0.969 | 0.946 | 0.872 | 1 |
| OAS1 SIGLEC1 OLAH FAM20A RETN MMP8 | 0.875 | 0.865 | 0.885 | 0.907 | 0.842 | 0.972 | 0.967 | 0.928 | 1 |
| IFI27 IFIT1 SIGLEC1 ISG15 HERC6 MMP8 | 0.875 | 0.866 | 0.884 | 0.905 | 0.847 | 0.963 | 0.908 | 0.832 | 0.983 |
| IFIT1 ISG15 HERC6 IL1R2 FAM20A RETN | 0.875 | 0.865 | 0.884 | 0.906 | 0.846 | 0.966 | 0.934 | 0.851 | 1 |
| IFI44L SIGLEC1 ISG15 HERC6 FAM20A RETN | 0.875 | 0.865 | 0.885 | 0.904 | 0.837 | 0.970 | 0.955 | 0.890 | 1 |
| RSAD2 IFI27 IFI44L SIGLEC1 SLC1A2 RETN | 0.875 | 0.866 | 0.884 | 0.914 | 0.858 | 0.970 | 0.952 | 0.896 | 1 |
| OAS1 IFIT1 SIGLEC1 HERC6 IL1R2 RETN | 0.875 | 0.866 | 0.884 | 0.905 | 0.843 | 0.968 | 0.952 | 0.907 | 0.997 |
| OAS1 SIGLEC1 HERC6 IL1R2 RETN MMP8 | 0.875 | 0.865 | 0.884 | 0.902 | 0.838 | 0.966 | 0.967 | 0.933 | 1 |
| OAS1 ISG15 HERC6 IL1R2 FAM20A MMP8 | 0.875 | 0.865 | 0.884 | 0.907 | 0.848 | 0.967 | 0.932 | 0.849 | 1 |
| RSAD2 HERC6 IL1R2 OLAH FAM20A MMP8 | 0.875 | 0.865 | 0.884 | 0.906 | 0.846 | 0.966 | 0.934 | 0.854 | 1 |
| IFI27 IFIT1 IFI44L ISG15 HERC6 FAM20A | 0.875 | 0.865 | 0.885 | 0.902 | 0.838 | 0.966 | 0.924 | 0.840 | 1 |
| IFI27 IFI44L ISG15 HERC6 FAM20A MMP8 | 0.875 | 0.865 | 0.885 | 0.907 | 0.845 | 0.969 | 0.932 | 0.851 | 1 |
| SIGLEC1 HERC6 SLC1A2 OLAH FAM20A RETN | 0.875 | 0.864 | 0.885 | 0.914 | 0.856 | 0.971 | 0.947 | 0.874 | 1 |
| RSAD2 IFIT1 SIGLEC1 ISG15 FAM20A RETN | 0.875 | 0.865 | 0.884 | 0.902 | 0.833 | 0.971 | 0.948 | 0.888 | 1 |
| IFI44L HERC6 SLC1A2 IL1R2 OLAH FAM20A | 0.875 | 0.865 | 0.885 | 0.914 | 0.860 | 0.968 | 0.929 | 0.846 | 1 |
| IFIT1 SIGLEC1 HERC6 SLC1A2 OLAH MMP8 | 0.875 | 0.865 | 0.885 | 0.913 | 0.858 | 0.968 | 0.947 | 0.898 | 0.996 |
| RSAD2 SIGLEC1 SLC1A2 IL1R2 OLAH MMP8 | 0.875 | 0.866 | 0.884 | 0.912 | 0.858 | 0.966 | 0.966 | 0.931 | 1 |
| IFI27 OAS1 ISG15 SLC1A2 IL1R2 MMP8 | 0.875 | 0.867 | 0.883 | 0.921 | 0.875 | 0.967 | 0.923 | 0.853 | 0.992 |
| OAS1 IFIT1 SIGLEC1 SLC1A2 FAM20A RETN | 0.875 | 0.865 | 0.884 | 0.910 | 0.849 | 0.970 | 0.943 | 0.870 | 1 |
| OAS1 SIGLEC1 ISG15 HERC6 OLAH FAM20A | 0.875 | 0.865 | 0.885 | 0.906 | 0.843 | 0.969 | 0.955 | 0.902 | 1 |
| IFI44L SIGLEC1 HERC6 OLAH FAM20A RETN | 0.875 | 0.865 | 0.885 | 0.904 | 0.837 | 0.970 | 0.954 | 0.893 | 1 |
| RSAD2 ISG15 HERC6 IL1R2 FAM20A RETN | 0.875 | 0.865 | 0.884 | 0.906 | 0.846 | 0.966 | 0.934 | 0.852 | 1 |
| OAS1 IFI44L SIGLEC1 HERC6 OLAH FAM20A | 0.875 | 0.865 | 0.884 | 0.905 | 0.841 | 0.969 | 0.954 | 0.899 | 1 |
| OAS1 SIGLEC1 ISG15 OLAH FAM20A MMP8 | 0.875 | 0.864 | 0.885 | 0.901 | 0.833 | 0.970 | 0.964 | 0.922 | 1 |
| ISG15 HERC6 IL1R2 OLAH FAM20A RETN | 0.874 | 0.865 | 0.884 | 0.906 | 0.845 | 0.967 | 0.937 | 0.862 | 1 |
| IFI27 SIGLEC1 ISG15 HERC6 SLC1A2 RETN | 0.874 | 0.865 | 0.884 | 0.910 | 0.848 | 0.972 | 0.952 | 0.895 | 1 |
| RSAD2 IFIT1 ISG15 HERC6 IL1R2 FAM20A | 0.874 | 0.865 | 0.884 | 0.906 | 0.846 | 0.966 | 0.934 | 0.855 | 1 |
| IFIT1 SIGLEC1 SLC1A2 FAM20A RETN MMP8 | 0.874 | 0.865 | 0.884 | 0.914 | 0.856 | 0.971 | 0.948 | 0.878 | 1 |
| IFIT1 IFI44L HERC6 IL1R2 FAM20A MMP8 | 0.874 | 0.865 | 0.883 | 0.908 | 0.849 | 0.968 | 0.934 | 0.851 | 1 |
| OAS1 IFI44L ISG15 HERC6 IL1R2 FAM20A | 0.874 | 0.865 | 0.884 | 0.907 | 0.847 | 0.967 | 0.938 | 0.861 | 1 |
| RSAD2 IFI27 IFI44L SIGLEC1 RETN MMP8 | 0.874 | 0.865 | 0.884 | 0.910 | 0.849 | 0.971 | 0.952 | 0.895 | 1 |
| RSAD2 SIGLEC1 OLAH FAM20A RETN MMP8 | 0.874 | 0.865 | 0.884 | 0.908 | 0.844 | 0.972 | 0.963 | 0.920 | 1 |
| OAS1 ISG15 HERC6 IL1R2 FAM20A RETN | 0.874 | 0.864 | 0.884 | 0.907 | 0.847 | 0.967 | 0.934 | 0.852 | 1 |
| RSAD2 IFI27 IFIT1 FAM20A RETN MMP8 | 0.874 | 0.864 | 0.884 | 0.912 | 0.850 | 0.975 | 0.943 | 0.866 | 1 |
| RSAD2 IFI27 SIGLEC1 ISG15 SLC1A2 RETN | 0.874 | 0.865 | 0.883 | 0.914 | 0.857 | 0.971 | 0.951 | 0.893 | 1 |
| RSAD2 IFIT1 SIGLEC1 ISG15 SLC1A2 FAM20A | 0.874 | 0.865 | 0.884 | 0.907 | 0.846 | 0.968 | 0.946 | 0.874 | 1 |
| IFI27 IFIT1 SIGLEC1 SLC1A2 RETN MMP8 | 0.874 | 0.865 | 0.883 | 0.915 | 0.859 | 0.971 | 0.955 | 0.900 | 1 |
| RSAD2 SIGLEC1 ISG15 HERC6 SLC1A2 FAM20A | 0.874 | 0.864 | 0.884 | 0.910 | 0.853 | 0.967 | 0.940 | 0.864 | 1 |
| OAS1 IFIT1 SIGLEC1 HERC6 IL1R2 MMP8 | 0.874 | 0.866 | 0.883 | 0.904 | 0.846 | 0.962 | 0.958 | 0.916 | 1.000 |
| OAS1 IFIT1 SIGLEC1 HERC6 SLC1A2 OLAH | 0.874 | 0.865 | 0.884 | 0.912 | 0.857 | 0.968 | 0.945 | 0.895 | 0.995 |
| OAS1 IFIT1 SIGLEC1 ISG15 HERC6 RETN | 0.874 | 0.865 | 0.884 | 0.902 | 0.839 | 0.966 | 0.932 | 0.873 | 0.990 |
| IFI27 OAS1 IFI44L ISG15 SLC1A2 IL1R2 | 0.874 | 0.866 | 0.883 | 0.922 | 0.877 | 0.968 | 0.930 | 0.866 | 0.994 |
| IFI27 OAS1 ISG15 FAM20A RETN MMP8 | 0.874 | 0.864 | 0.884 | 0.910 | 0.848 | 0.972 | 0.943 | 0.866 | 1 |
| IFIT1 ISG15 HERC6 IL1R2 FAM20A MMP8 | 0.874 | 0.865 | 0.884 | 0.906 | 0.846 | 0.966 | 0.937 | 0.859 | 1 |
| IFI44L SIGLEC1 HERC6 IL1R2 RETN MMP8 | 0.874 | 0.865 | 0.884 | 0.901 | 0.837 | 0.965 | 0.963 | 0.925 | 1 |
| RSAD2 OAS1 SIGLEC1 ISG15 OLAH FAM20A | 0.874 | 0.864 | 0.884 | 0.898 | 0.827 | 0.969 | 0.959 | 0.911 | 1 |
| IFIT1 SIGLEC1 ISG15 SLC1A2 OLAH MMP8 | 0.874 | 0.865 | 0.883 | 0.913 | 0.857 | 0.968 | 0.952 | 0.907 | 0.997 |
| IFI27 OAS1 IFI44L SIGLEC1 HERC6 MMP8 | 0.874 | 0.866 | 0.883 | 0.906 | 0.850 | 0.962 | 0.910 | 0.835 | 0.985 |
| RSAD2 OAS1 IFI44L HERC6 IL1R2 FAM20A | 0.874 | 0.865 | 0.883 | 0.907 | 0.846 | 0.968 | 0.936 | 0.857 | 1 |
| OAS1 ISG15 HERC6 IL1R2 OLAH FAM20A | 0.874 | 0.864 | 0.884 | 0.906 | 0.846 | 0.966 | 0.936 | 0.860 | 1 |
| OAS1 IFI44L HERC6 IL1R2 FAM20A MMP8 | 0.874 | 0.865 | 0.883 | 0.909 | 0.849 | 0.969 | 0.934 | 0.851 | 1 |
| SIGLEC1 ISG15 HERC6 OLAH FAM20A RETN | 0.874 | 0.864 | 0.884 | 0.900 | 0.833 | 0.968 | 0.958 | 0.902 | 1 |
| SIGLEC1 ISG15 SLC1A2 IL1R2 OLAH MMP8 | 0.874 | 0.866 | 0.883 | 0.913 | 0.859 | 0.966 | 0.965 | 0.929 | 1 |
| RSAD2 SIGLEC1 HERC6 IL1R2 RETN MMP8 | 0.874 | 0.864 | 0.884 | 0.902 | 0.838 | 0.966 | 0.961 | 0.921 | 1 |
| IFI44L HERC6 SLC1A2 OLAH FAM20A MMP8 | 0.874 | 0.864 | 0.881 | 0.912 | 0.855 | 0.969 | 0.934 | 0.859 | 1 |
| RSAD2 ISG15 HERC6 IL1R2 FAM20A MMP8 | 0.874 | 0.864 | 0.884 | 0.906 | 0.847 | 0.966 | 0.930 | 0.846 | 1 |
| OAS1 IFIT1 IFI44L SIGLEC1 HERC6 IL1R2 | 0.874 | 0.865 | 0.883 | 0.903 | 0.843 | 0.962 | 0.955 | 0.912 | 0.999 |
| RSAD2 OAS1 SIGLEC1 HERC6 FAM20A RETN | 0.874 | 0.865 | 0.883 | 0.907 | 0.844 | 0.969 | 0.943 | 0.876 | 1 |
| IFIT1 SIGLEC1 SLC1A2 IL1R2 OLAH MMP8 | 0.874 | 0.865 | 0.883 | 0.911 | 0.856 | 0.966 | 0.965 | 0.929 | 1 |
| IFIT1 SIGLEC1 HERC6 OLAH RETN MMP8 | 0.874 | 0.864 | 0.884 | 0.901 | 0.836 | 0.967 | 0.958 | 0.917 | 0.999 |
| RSAD2 OAS1 SIGLEC1 ISG15 HERC6 FAM20A | 0.874 | 0.864 | 0.883 | 0.902 | 0.838 | 0.967 | 0.940 | 0.869 | 1 |
| IFIT1 SIGLEC1 ISG15 SLC1A2 FAM20A MMP8 | 0.874 | 0.864 | 0.883 | 0.905 | 0.844 | 0.966 | 0.940 | 0.867 | 1 |
| IFIT1 IFI44L SIGLEC1 HERC6 IL1R2 MMP8 | 0.874 | 0.865 | 0.883 | 0.902 | 0.843 | 0.961 | 0.961 | 0.921 | 1 |
| RSAD2 IFI27 HERC6 SLC1A2 RETN MMP8 | 0.874 | 0.864 | 0.883 | 0.911 | 0.852 | 0.970 | 0.929 | 0.858 | 1 |
| IFI27 IFIT1 ISG15 HERC6 SLC1A2 RETN | 0.874 | 0.864 | 0.883 | 0.911 | 0.852 | 0.971 | 0.939 | 0.870 | 1 |
| OAS1 IFI44L SIGLEC1 HERC6 IL1R2 MMP8 | 0.874 | 0.865 | 0.882 | 0.902 | 0.843 | 0.961 | 0.961 | 0.921 | 1 |
| IFI27 OAS1 IFI44L SLC1A2 FAM20A RETN | 0.874 | 0.864 | 0.884 | 0.911 | 0.848 | 0.973 | 0.939 | 0.857 | 1 |
| RSAD2 SIGLEC1 HERC6 SLC1A2 IL1R2 OLAH | 0.874 | 0.864 | 0.883 | 0.911 | 0.855 | 0.966 | 0.955 | 0.912 | 0.999 |
| SIGLEC1 HERC6 SLC1A2 IL1R2 OLAH MMP8 | 0.874 | 0.864 | 0.883 | 0.911 | 0.857 | 0.966 | 0.965 | 0.929 | 1 |
| IFI44L SLC1A2 IL1R2 OLAH FAM20A RETN | 0.874 | 0.864 | 0.883 | 0.913 | 0.858 | 0.968 | 0.927 | 0.849 | 1 |
| IFI44L SIGLEC1 ISG15 SLC1A2 IL1R2 OLAH | 0.874 | 0.865 | 0.882 | 0.914 | 0.859 | 0.968 | 0.962 | 0.924 | 1 |
| OAS1 SIGLEC1 HERC6 SLC1A2 OLAH MMP8 | 0.874 | 0.864 | 0.883 | 0.911 | 0.856 | 0.967 | 0.954 | 0.910 | 0.999 |
| RSAD2 IFIT1 SIGLEC1 ISG15 FAM20A MMP8 | 0.874 | 0.864 | 0.883 | 0.899 | 0.830 | 0.969 | 0.943 | 0.877 | 1 |
| IFIT1 SLC1A2 IL1R2 OLAH FAM20A MMP8 | 0.874 | 0.865 | 0.882 | 0.914 | 0.861 | 0.967 | 0.935 | 0.858 | 1 |
| OAS1 IFIT1 IL1R2 OLAH FAM20A MMP8 | 0.874 | 0.865 | 0.883 | 0.906 | 0.844 | 0.968 | 0.942 | 0.870 | 1 |
| RSAD2 OAS1 SIGLEC1 HERC6 IL1R2 MMP8 | 0.874 | 0.864 | 0.883 | 0.904 | 0.845 | 0.962 | 0.963 | 0.925 | 1 |
| IFIT1 IFI44L SIGLEC1 HERC6 IL1R2 RETN | 0.874 | 0.864 | 0.883 | 0.905 | 0.842 | 0.967 | 0.952 | 0.907 | 0.997 |
| OAS1 IFIT1 SIGLEC1 SLC1A2 OLAH MMP8 | 0.874 | 0.864 | 0.883 | 0.911 | 0.855 | 0.967 | 0.952 | 0.907 | 0.997 |
| OAS1 SIGLEC1 HERC6 OLAH FAM20A RETN | 0.874 | 0.864 | 0.883 | 0.906 | 0.842 | 0.969 | 0.954 | 0.900 | 1 |
| RSAD2 IFIT1 SIGLEC1 SLC1A2 FAM20A MMP8 | 0.874 | 0.864 | 0.883 | 0.907 | 0.846 | 0.967 | 0.946 | 0.874 | 1 |
| RSAD2 OAS1 SIGLEC1 SLC1A2 OLAH MMP8 | 0.873 | 0.864 | 0.883 | 0.911 | 0.855 | 0.966 | 0.957 | 0.913 | 1.000 |
| OAS1 SIGLEC1 SLC1A2 IL1R2 OLAH MMP8 | 0.873 | 0.864 | 0.883 | 0.912 | 0.858 | 0.966 | 0.966 | 0.931 | 1 |
| IFI27 IFIT1 HERC6 SLC1A2 RETN MMP8 | 0.873 | 0.864 | 0.883 | 0.911 | 0.851 | 0.971 | 0.927 | 0.853 | 1 |
| IFIT1 SIGLEC1 ISG15 HERC6 SLC1A2 OLAH | 0.873 | 0.864 | 0.883 | 0.912 | 0.857 | 0.967 | 0.943 | 0.893 | 0.994 |
| RSAD2 IFI27 SIGLEC1 SLC1A2 RETN MMP8 | 0.873 | 0.864 | 0.883 | 0.914 | 0.857 | 0.971 | 0.954 | 0.898 | 1 |
| IFIT1 SIGLEC1 ISG15 HERC6 IL1R2 RETN | 0.873 | 0.864 | 0.882 | 0.905 | 0.843 | 0.967 | 0.952 | 0.907 | 0.998 |
| OAS1 IFIT1 HERC6 IL1R2 OLAH FAM20A | 0.873 | 0.864 | 0.883 | 0.906 | 0.844 | 0.967 | 0.938 | 0.864 | 1 |
| IFI44L SIGLEC1 HERC6 IL1R2 OLAH MMP8 | 0.873 | 0.864 | 0.882 | 0.903 | 0.844 | 0.962 | 0.963 | 0.924 | 1 |
| RSAD2 OAS1 SIGLEC1 HERC6 SLC1A2 FAM20A | 0.873 | 0.864 | 0.883 | 0.909 | 0.852 | 0.965 | 0.939 | 0.863 | 1 |
| IFI44L HERC6 IL1R2 OLAH FAM20A RETN | 0.873 | 0.864 | 0.883 | 0.905 | 0.844 | 0.967 | 0.938 | 0.864 | 1 |
| IFI27 OAS1 IFI44L ISG15 HERC6 FAM20A | 0.873 | 0.863 | 0.883 | 0.904 | 0.841 | 0.968 | 0.927 | 0.844 | 1 |
| IFIT1 IFI44L SIGLEC1 ISG15 HERC6 IL1R2 | 0.873 | 0.865 | 0.882 | 0.904 | 0.845 | 0.962 | 0.954 | 0.910 | 0.999 |
| RSAD2 OAS1 IFIT1 HERC6 IL1R2 FAM20A | 0.873 | 0.864 | 0.883 | 0.904 | 0.841 | 0.966 | 0.936 | 0.855 | 1 |
| RSAD2 OAS1 IFIT1 SIGLEC1 HERC6 IL1R2 | 0.873 | 0.864 | 0.882 | 0.904 | 0.847 | 0.962 | 0.958 | 0.916 | 0.999 |
| RSAD2 IFI44L SLC1A2 OLAH FAM20A MMP8 | 0.873 | 0.864 | 0.883 | 0.914 | 0.857 | 0.970 | 0.925 | 0.851 | 0.999 |
| IFIT1 SIGLEC1 HERC6 SLC1A2 IL1R2 OLAH | 0.873 | 0.864 | 0.883 | 0.913 | 0.858 | 0.967 | 0.952 | 0.908 | 0.997 |
| IFI27 OAS1 SIGLEC1 ISG15 HERC6 MMP8 | 0.873 | 0.865 | 0.882 | 0.908 | 0.853 | 0.963 | 0.917 | 0.846 | 0.989 |
| IFI44L SLC1A2 OLAH FAM20A RETN MMP8 | 0.873 | 0.864 | 0.883 | 0.914 | 0.856 | 0.971 | 0.932 | 0.859 | 1 |
| RSAD2 IFI27 OAS1 ISG15 HERC6 FAM20A | 0.873 | 0.863 | 0.883 | 0.908 | 0.847 | 0.968 | 0.928 | 0.846 | 1 |
| RSAD2 IFI27 IFI44L SLC1A2 FAM20A RETN | 0.873 | 0.863 | 0.883 | 0.910 | 0.847 | 0.973 | 0.938 | 0.857 | 1 |
| RSAD2 OAS1 IFIT1 IL1R2 FAM20A MMP8 | 0.873 | 0.864 | 0.882 | 0.907 | 0.845 | 0.969 | 0.946 | 0.873 | 1 |
| IFIT1 IFI44L SIGLEC1 HERC6 SLC1A2 OLAH | 0.873 | 0.864 | 0.883 | 0.912 | 0.857 | 0.967 | 0.940 | 0.888 | 0.993 |
| RSAD2 IFIT1 HERC6 IL1R2 FAM20A MMP8 | 0.873 | 0.864 | 0.882 | 0.904 | 0.843 | 0.965 | 0.937 | 0.859 | 1 |
| RSAD2 IFIT1 SIGLEC1 HERC6 SLC1A2 OLAH | 0.873 | 0.864 | 0.883 | 0.912 | 0.857 | 0.967 | 0.943 | 0.893 | 0.994 |
| RSAD2 SIGLEC1 HERC6 SLC1A2 FAM20A RETN | 0.873 | 0.863 | 0.883 | 0.911 | 0.854 | 0.969 | 0.940 | 0.864 | 1 |
| IFIT1 SIGLEC1 ISG15 SLC1A2 IL1R2 OLAH | 0.873 | 0.864 | 0.882 | 0.913 | 0.858 | 0.968 | 0.962 | 0.924 | 1 |
| RSAD2 OAS1 IFI44L SIGLEC1 OLAH FAM20A | 0.873 | 0.863 | 0.883 | 0.898 | 0.827 | 0.970 | 0.957 | 0.908 | 1 |
| IFIT1 HERC6 IL1R2 FAM20A RETN MMP8 | 0.873 | 0.864 | 0.883 | 0.907 | 0.845 | 0.970 | 0.940 | 0.860 | 1 |
| RSAD2 IFI44L SIGLEC1 HERC6 IL1R2 MMP8 | 0.873 | 0.864 | 0.882 | 0.902 | 0.842 | 0.962 | 0.960 | 0.919 | 1 |
| RSAD2 IFI27 OAS1 SIGLEC1 HERC6 MMP8 | 0.873 | 0.864 | 0.882 | 0.905 | 0.849 | 0.962 | 0.918 | 0.846 | 0.991 |
| IFI44L ISG15 HERC6 SLC1A2 IL1R2 FAM20A | 0.873 | 0.863 | 0.883 | 0.912 | 0.859 | 0.965 | 0.928 | 0.839 | 1 |
| OAS1 IFIT1 IFI44L SIGLEC1 SLC1A2 OLAH | 0.873 | 0.864 | 0.882 | 0.912 | 0.856 | 0.968 | 0.943 | 0.892 | 0.995 |
| IFIT1 SIGLEC1 ISG15 SLC1A2 FAM20A RETN | 0.873 | 0.864 | 0.882 | 0.908 | 0.848 | 0.968 | 0.940 | 0.868 | 1 |
| SIGLEC1 ISG15 HERC6 SLC1A2 OLAH MMP8 | 0.873 | 0.863 | 0.882 | 0.910 | 0.853 | 0.966 | 0.951 | 0.905 | 0.997 |
| IFIT1 SIGLEC1 HERC6 SLC1A2 OLAH RETN | 0.873 | 0.863 | 0.883 | 0.914 | 0.858 | 0.969 | 0.943 | 0.893 | 0.994 |
| OAS1 IFI44L HERC6 IL1R2 OLAH FAM20A | 0.873 | 0.863 | 0.882 | 0.906 | 0.845 | 0.967 | 0.939 | 0.867 | 1 |
| RSAD2 OAS1 SIGLEC1 FAM20A RETN MMP8 | 0.873 | 0.863 | 0.883 | 0.903 | 0.836 | 0.971 | 0.955 | 0.893 | 1 |
| IFIT1 IFI44L SIGLEC1 ISG15 SLC1A2 OLAH | 0.873 | 0.864 | 0.882 | 0.912 | 0.856 | 0.968 | 0.943 | 0.892 | 0.995 |
| HERC6 IL1R2 OLAH FAM20A RETN MMP8 | 0.873 | 0.863 | 0.883 | 0.909 | 0.850 | 0.968 | 0.939 | 0.862 | 1 |
| IFI44L SIGLEC1 ISG15 HERC6 IL1R2 MMP8 | 0.873 | 0.864 | 0.881 | 0.902 | 0.843 | 0.961 | 0.960 | 0.919 | 1 |
| IFI27 IFIT1 IFI44L HERC6 SLC1A2 RETN | 0.873 | 0.863 | 0.883 | 0.910 | 0.849 | 0.971 | 0.938 | 0.869 | 1 |
| OAS1 SIGLEC1 HERC6 SLC1A2 IL1R2 OLAH | 0.873 | 0.863 | 0.883 | 0.912 | 0.858 | 0.966 | 0.959 | 0.917 | 1 |
| SIGLEC1 HERC6 SLC1A2 FAM20A RETN MMP8 | 0.873 | 0.863 | 0.883 | 0.904 | 0.840 | 0.968 | 0.953 | 0.880 | 1 |
| IFI44L ISG15 SLC1A2 IL1R2 FAM20A MMP8 | 0.873 | 0.864 | 0.881 | 0.911 | 0.859 | 0.963 | 0.928 | 0.840 | 1 |
| RSAD2 OAS1 IFIT1 SIGLEC1 SLC1A2 OLAH | 0.873 | 0.863 | 0.882 | 0.911 | 0.855 | 0.968 | 0.952 | 0.905 | 0.999 |
| RSAD2 IFIT1 SIGLEC1 SLC1A2 OLAH MMP8 | 0.873 | 0.864 | 0.882 | 0.911 | 0.855 | 0.967 | 0.954 | 0.910 | 0.998 |
| IFI27 IFI44L HERC6 SLC1A2 RETN MMP8 | 0.873 | 0.863 | 0.882 | 0.909 | 0.848 | 0.970 | 0.930 | 0.857 | 1 |
| RSAD2 IFI27 IFIT1 ISG15 HERC6 FAM20A | 0.873 | 0.863 | 0.883 | 0.907 | 0.847 | 0.968 | 0.926 | 0.843 | 1 |
| IFI27 OAS1 ISG15 SLC1A2 FAM20A RETN | 0.873 | 0.863 | 0.883 | 0.910 | 0.849 | 0.971 | 0.940 | 0.860 | 1 |
| OAS1 IFIT1 IFI44L HERC6 IL1R2 FAM20A | 0.873 | 0.864 | 0.882 | 0.906 | 0.844 | 0.967 | 0.936 | 0.856 | 1 |
| IFIT1 SIGLEC1 ISG15 HERC6 IL1R2 MMP8 | 0.873 | 0.864 | 0.881 | 0.904 | 0.847 | 0.962 | 0.957 | 0.913 | 1 |
| OAS1 IFIT1 SIGLEC1 ISG15 HERC6 IL1R2 | 0.873 | 0.864 | 0.882 | 0.905 | 0.849 | 0.962 | 0.951 | 0.904 | 0.998 |
| RSAD2 HERC6 SLC1A2 IL1R2 OLAH FAM20A | 0.873 | 0.863 | 0.883 | 0.914 | 0.859 | 0.968 | 0.928 | 0.844 | 1 |
| ISG15 HERC6 SLC1A2 IL1R2 OLAH FAM20A | 0.873 | 0.863 | 0.883 | 0.911 | 0.857 | 0.966 | 0.926 | 0.842 | 1 |
| IFI27 IFIT1 ISG15 SLC1A2 FAM20A RETN | 0.873 | 0.863 | 0.883 | 0.908 | 0.847 | 0.969 | 0.940 | 0.859 | 1 |
| OAS1 ISG15 HERC6 SLC1A2 IL1R2 FAM20A | 0.873 | 0.863 | 0.883 | 0.911 | 0.858 | 0.965 | 0.925 | 0.835 | 1 |
| IFI44L SIGLEC1 SLC1A2 IL1R2 OLAH RETN | 0.873 | 0.864 | 0.882 | 0.913 | 0.858 | 0.968 | 0.962 | 0.922 | 1 |
| RSAD2 SIGLEC1 HERC6 SLC1A2 OLAH MMP8 | 0.873 | 0.863 | 0.882 | 0.912 | 0.857 | 0.967 | 0.950 | 0.904 | 0.996 |
| RSAD2 IFI27 ISG15 FAM20A RETN MMP8 | 0.873 | 0.862 | 0.883 | 0.909 | 0.847 | 0.971 | 0.945 | 0.867 | 1 |
| RSAD2 OAS1 SIGLEC1 HERC6 SLC1A2 OLAH | 0.873 | 0.863 | 0.882 | 0.912 | 0.856 | 0.967 | 0.951 | 0.904 | 0.998 |
| RSAD2 IFIT1 SIGLEC1 SLC1A2 FAM20A RETN | 0.873 | 0.863 | 0.882 | 0.908 | 0.848 | 0.969 | 0.946 | 0.876 | 1 |
| RSAD2 IFI44L HERC6 IL1R2 FAM20A MMP8 | 0.873 | 0.863 | 0.882 | 0.909 | 0.850 | 0.968 | 0.932 | 0.846 | 1 |
| IFI27 OAS1 IFIT1 IFI44L HERC6 FAM20A | 0.873 | 0.863 | 0.882 | 0.903 | 0.838 | 0.968 | 0.927 | 0.845 | 1 |
| IFI27 IFI44L ISG15 HERC6 SLC1A2 RETN | 0.873 | 0.863 | 0.882 | 0.910 | 0.849 | 0.971 | 0.939 | 0.870 | 1 |
| RSAD2 OAS1 SIGLEC1 OLAH FAM20A RETN | 0.873 | 0.863 | 0.883 | 0.896 | 0.824 | 0.969 | 0.958 | 0.911 | 1 |
| RSAD2 OAS1 SIGLEC1 HERC6 IL1R2 RETN | 0.873 | 0.863 | 0.882 | 0.901 | 0.837 | 0.965 | 0.960 | 0.919 | 1 |
| IFI44L ISG15 HERC6 SLC1A2 OLAH FAM20A | 0.873 | 0.863 | 0.883 | 0.912 | 0.855 | 0.969 | 0.929 | 0.851 | 1 |
| IFIT1 IFI44L SLC1A2 IL1R2 OLAH FAM20A | 0.873 | 0.863 | 0.882 | 0.912 | 0.855 | 0.968 | 0.926 | 0.849 | 1 |
| RSAD2 SIGLEC1 HERC6 SLC1A2 FAM20A MMP8 | 0.873 | 0.863 | 0.882 | 0.908 | 0.851 | 0.965 | 0.943 | 0.868 | 1 |
| IFI44L HERC6 IL1R2 FAM20A RETN MMP8 | 0.872 | 0.863 | 0.882 | 0.909 | 0.848 | 0.970 | 0.938 | 0.857 | 1 |
| RSAD2 IFI44L HERC6 IL1R2 OLAH FAM20A | 0.872 | 0.863 | 0.882 | 0.906 | 0.846 | 0.966 | 0.937 | 0.863 | 1 |
| IFIT1 IFI44L HERC6 OLAH FAM20A MMP8 | 0.872 | 0.863 | 0.882 | 0.901 | 0.837 | 0.965 | 0.937 | 0.869 | 1 |
| IFI44L HERC6 SLC1A2 IL1R2 FAM20A MMP8 | 0.872 | 0.863 | 0.882 | 0.916 | 0.866 | 0.967 | 0.923 | 0.827 | 1 |
| IFIT1 IFI44L HERC6 IL1R2 FAM20A RETN | 0.872 | 0.863 | 0.882 | 0.906 | 0.844 | 0.968 | 0.936 | 0.855 | 1 |
| IFIT1 SIGLEC1 HERC6 IL1R2 OLAH MMP8 | 0.872 | 0.863 | 0.881 | 0.906 | 0.849 | 0.964 | 0.958 | 0.915 | 1.000 |
| IFIT1 IFI44L SIGLEC1 SLC1A2 OLAH RETN | 0.872 | 0.863 | 0.881 | 0.915 | 0.860 | 0.971 | 0.939 | 0.883 | 0.995 |
| RSAD2 OAS1 SIGLEC1 SLC1A2 IL1R2 OLAH | 0.872 | 0.863 | 0.882 | 0.913 | 0.857 | 0.968 | 0.966 | 0.929 | 1 |
| IFI44L SIGLEC1 ISG15 FAM20A RETN MMP8 | 0.872 | 0.862 | 0.882 | 0.901 | 0.832 | 0.971 | 0.959 | 0.897 | 1 |
| RSAD2 IFIT1 IFI44L SIGLEC1 HERC6 IL1R2 | 0.872 | 0.864 | 0.881 | 0.900 | 0.839 | 0.962 | 0.955 | 0.913 | 0.998 |
| RSAD2 OAS1 IFI44L SIGLEC1 HERC6 IL1R2 | 0.872 | 0.863 | 0.881 | 0.901 | 0.840 | 0.962 | 0.961 | 0.921 | 1 |
| IFIT1 SIGLEC1 HERC6 SLC1A2 IL1R2 RETN | 0.872 | 0.863 | 0.881 | 0.910 | 0.855 | 0.965 | 0.951 | 0.906 | 0.996 |
| IFI44L SIGLEC1 ISG15 SLC1A2 OLAH RETN | 0.872 | 0.863 | 0.881 | 0.912 | 0.856 | 0.969 | 0.948 | 0.898 | 0.998 |
| IFIT1 IFI44L ISG15 SLC1A2 OLAH FAM20A | 0.872 | 0.863 | 0.882 | 0.909 | 0.850 | 0.968 | 0.920 | 0.843 | 0.997 |
| OAS1 SIGLEC1 ISG15 HERC6 SLC1A2 OLAH | 0.872 | 0.863 | 0.882 | 0.912 | 0.857 | 0.967 | 0.951 | 0.904 | 0.998 |
| OAS1 IFIT1 SIGLEC1 SLC1A2 IL1R2 OLAH | 0.872 | 0.863 | 0.882 | 0.912 | 0.857 | 0.967 | 0.962 | 0.923 | 1 |
| RSAD2 IFIT1 SIGLEC1 HERC6 IL1R2 MMP8 | 0.872 | 0.863 | 0.881 | 0.903 | 0.844 | 0.962 | 0.958 | 0.916 | 0.999 |
| IFIT1 IFI44L HERC6 OLAH FAM20A RETN | 0.872 | 0.863 | 0.882 | 0.903 | 0.840 | 0.967 | 0.935 | 0.864 | 1 |
| OAS1 HERC6 IL1R2 FAM20A RETN MMP8 | 0.872 | 0.863 | 0.882 | 0.906 | 0.845 | 0.968 | 0.938 | 0.857 | 1 |
| OAS1 IFIT1 SIGLEC1 ISG15 SLC1A2 OLAH | 0.872 | 0.863 | 0.882 | 0.912 | 0.856 | 0.968 | 0.945 | 0.894 | 0.995 |
| IFI27 IFI44L SIGLEC1 SLC1A2 RETN MMP8 | 0.872 | 0.863 | 0.881 | 0.915 | 0.858 | 0.972 | 0.954 | 0.898 | 1 |
| RSAD2 SIGLEC1 ISG15 HERC6 FAM20A MMP8 | 0.872 | 0.863 | 0.882 | 0.903 | 0.838 | 0.967 | 0.943 | 0.875 | 1 |
| OAS1 IFIT1 SIGLEC1 HERC6 OLAH RETN | 0.872 | 0.863 | 0.882 | 0.900 | 0.836 | 0.965 | 0.941 | 0.890 | 0.993 |
| IFIT1 SIGLEC1 ISG15 HERC6 IL1R2 OLAH | 0.872 | 0.863 | 0.881 | 0.908 | 0.850 | 0.966 | 0.950 | 0.904 | 0.996 |
| IFI27 SIGLEC1 HERC6 SLC1A2 RETN MMP8 | 0.872 | 0.862 | 0.882 | 0.910 | 0.847 | 0.972 | 0.953 | 0.896 | 1 |
| OAS1 SIGLEC1 HERC6 SLC1A2 FAM20A RETN | 0.872 | 0.862 | 0.882 | 0.910 | 0.852 | 0.967 | 0.943 | 0.871 | 1 |
| RSAD2 SIGLEC1 HERC6 IL1R2 OLAH MMP8 | 0.872 | 0.863 | 0.882 | 0.905 | 0.847 | 0.963 | 0.960 | 0.919 | 1 |
| RSAD2 IFIT1 SIGLEC1 SLC1A2 IL1R2 OLAH | 0.872 | 0.863 | 0.881 | 0.911 | 0.855 | 0.967 | 0.963 | 0.925 | 1 |
| OAS1 IFIT1 SIGLEC1 HERC6 SLC1A2 IL1R2 | 0.872 | 0.863 | 0.881 | 0.910 | 0.857 | 0.963 | 0.960 | 0.920 | 1.000 |
| RSAD2 OAS1 HERC6 IL1R2 FAM20A RETN | 0.872 | 0.862 | 0.882 | 0.905 | 0.842 | 0.967 | 0.935 | 0.853 | 1 |
| IFIT1 SIGLEC1 ISG15 HERC6 OLAH RETN | 0.872 | 0.863 | 0.882 | 0.901 | 0.837 | 0.966 | 0.948 | 0.900 | 0.995 |
| IFI27 OAS1 IFIT1 SLC1A2 FAM20A RETN | 0.872 | 0.862 | 0.882 | 0.908 | 0.848 | 0.969 | 0.939 | 0.858 | 1 |
| RSAD2 SIGLEC1 ISG15 SLC1A2 OLAH MMP8 | 0.872 | 0.863 | 0.881 | 0.912 | 0.857 | 0.968 | 0.953 | 0.909 | 0.998 |
| IFI44L SIGLEC1 SLC1A2 FAM20A RETN MMP8 | 0.872 | 0.862 | 0.882 | 0.903 | 0.839 | 0.967 | 0.953 | 0.882 | 1 |
| RSAD2 IFIT1 SIGLEC1 HERC6 IL1R2 RETN | 0.872 | 0.863 | 0.881 | 0.904 | 0.840 | 0.967 | 0.953 | 0.909 | 0.997 |
| IFI44L SIGLEC1 ISG15 OLAH FAM20A RETN | 0.872 | 0.862 | 0.882 | 0.897 | 0.826 | 0.969 | 0.961 | 0.916 | 1 |
| OAS1 SIGLEC1 ISG15 SLC1A2 OLAH MMP8 | 0.872 | 0.863 | 0.881 | 0.913 | 0.857 | 0.968 | 0.955 | 0.913 | 0.998 |
| OAS1 SIGLEC1 HERC6 IL1R2 OLAH MMP8 | 0.872 | 0.863 | 0.881 | 0.904 | 0.846 | 0.962 | 0.962 | 0.922 | 1 |
| OAS1 IFIT1 SIGLEC1 SLC1A2 OLAH RETN | 0.872 | 0.862 | 0.882 | 0.914 | 0.858 | 0.970 | 0.945 | 0.893 | 0.996 |
| OAS1 IFI44L SIGLEC1 HERC6 FAM20A RETN | 0.872 | 0.862 | 0.882 | 0.904 | 0.840 | 0.968 | 0.949 | 0.885 | 1 |
| RSAD2 IFI27 IFI44L HERC6 SLC1A2 FAM20A | 0.872 | 0.861 | 0.882 | 0.914 | 0.857 | 0.970 | 0.928 | 0.847 | 1 |
| IFIT1 IFI44L SLC1A2 IL1R2 FAM20A MMP8 | 0.872 | 0.863 | 0.881 | 0.912 | 0.859 | 0.964 | 0.930 | 0.843 | 1 |
| OAS1 SIGLEC1 ISG15 HERC6 IL1R2 RETN | 0.872 | 0.862 | 0.882 | 0.903 | 0.842 | 0.965 | 0.962 | 0.921 | 1 |
| OAS1 IFI44L SLC1A2 OLAH FAM20A MMP8 | 0.872 | 0.862 | 0.882 | 0.912 | 0.855 | 0.970 | 0.928 | 0.854 | 1 |
| SIGLEC1 ISG15 HERC6 SLC1A2 IL1R2 OLAH | 0.872 | 0.863 | 0.881 | 0.912 | 0.857 | 0.966 | 0.957 | 0.914 | 0.999 |
| OAS1 IFIT1 SIGLEC1 HERC6 IL1R2 OLAH | 0.872 | 0.863 | 0.881 | 0.907 | 0.848 | 0.965 | 0.951 | 0.905 | 0.997 |
| RSAD2 SIGLEC1 ISG15 SLC1A2 IL1R2 OLAH | 0.872 | 0.863 | 0.881 | 0.912 | 0.857 | 0.968 | 0.961 | 0.922 | 1 |
| OAS1 IFI44L SIGLEC1 ISG15 OLAH FAM20A | 0.872 | 0.862 | 0.882 | 0.898 | 0.826 | 0.970 | 0.961 | 0.915 | 1 |
| RSAD2 SLC1A2 IL1R2 OLAH FAM20A MMP8 | 0.872 | 0.863 | 0.881 | 0.914 | 0.862 | 0.966 | 0.929 | 0.844 | 1 |
| OAS1 SLC1A2 IL1R2 OLAH FAM20A MMP8 | 0.872 | 0.863 | 0.881 | 0.913 | 0.860 | 0.967 | 0.930 | 0.847 | 1 |
| SIGLEC1 SLC1A2 IL1R2 OLAH RETN MMP8 | 0.872 | 0.862 | 0.881 | 0.914 | 0.859 | 0.968 | 0.968 | 0.935 | 1 |
| IFI44L SIGLEC1 SLC1A2 OLAH RETN MMP8 | 0.872 | 0.862 | 0.881 | 0.908 | 0.850 | 0.967 | 0.962 | 0.923 | 1 |
| SIGLEC1 ISG15 SLC1A2 FAM20A RETN MMP8 | 0.872 | 0.862 | 0.882 | 0.903 | 0.840 | 0.967 | 0.953 | 0.882 | 1 |
| OAS1 IFI44L HERC6 SLC1A2 OLAH FAM20A | 0.872 | 0.861 | 0.882 | 0.911 | 0.854 | 0.967 | 0.924 | 0.844 | 1 |
| IFIT1 IFI44L ISG15 HERC6 OLAH FAM20A | 0.872 | 0.862 | 0.881 | 0.903 | 0.839 | 0.966 | 0.935 | 0.864 | 1 |
| RSAD2 ISG15 HERC6 SLC1A2 IL1R2 FAM20A | 0.872 | 0.862 | 0.881 | 0.909 | 0.855 | 0.963 | 0.924 | 0.832 | 1 |
| OAS1 IFI44L SIGLEC1 HERC6 IL1R2 RETN | 0.872 | 0.862 | 0.881 | 0.903 | 0.840 | 0.965 | 0.957 | 0.914 | 0.999 |
| IFI27 SIGLEC1 ISG15 SLC1A2 RETN MMP8 | 0.872 | 0.862 | 0.881 | 0.910 | 0.850 | 0.970 | 0.957 | 0.904 | 1 |
| OAS1 SIGLEC1 HERC6 SLC1A2 IL1R2 RETN | 0.872 | 0.862 | 0.881 | 0.911 | 0.857 | 0.966 | 0.957 | 0.913 | 1 |
| OAS1 IFI44L SIGLEC1 SLC1A2 OLAH MMP8 | 0.872 | 0.862 | 0.881 | 0.910 | 0.854 | 0.966 | 0.957 | 0.914 | 0.999 |
| OAS1 IFIT1 IFI44L SIGLEC1 HERC6 RETN | 0.872 | 0.862 | 0.881 | 0.900 | 0.833 | 0.967 | 0.923 | 0.862 | 0.984 |
| IFIT1 HERC6 IL1R2 OLAH FAM20A RETN | 0.872 | 0.862 | 0.881 | 0.905 | 0.844 | 0.967 | 0.936 | 0.861 | 1 |
| RSAD2 OAS1 SIGLEC1 HERC6 FAM20A MMP8 | 0.872 | 0.862 | 0.881 | 0.899 | 0.833 | 0.965 | 0.943 | 0.873 | 1 |
| RSAD2 IFI27 IFIT1 SIGLEC1 HERC6 MMP8 | 0.872 | 0.863 | 0.881 | 0.905 | 0.848 | 0.962 | 0.904 | 0.828 | 0.981 |
| IFI44L ISG15 SLC1A2 OLAH FAM20A RETN | 0.872 | 0.862 | 0.882 | 0.909 | 0.849 | 0.968 | 0.927 | 0.853 | 1 |
| IFI44L SIGLEC1 HERC6 SLC1A2 FAM20A RETN | 0.872 | 0.862 | 0.882 | 0.904 | 0.841 | 0.967 | 0.950 | 0.876 | 1 |
| IFI27 IFIT1 SIGLEC1 HERC6 SLC1A2 MMP8 | 0.872 | 0.863 | 0.881 | 0.910 | 0.856 | 0.964 | 0.917 | 0.848 | 0.987 |
| RSAD2 OAS1 IFIT1 SIGLEC1 HERC6 RETN | 0.872 | 0.862 | 0.881 | 0.899 | 0.832 | 0.966 | 0.927 | 0.868 | 0.987 |
| RSAD2 IFI27 OAS1 IFI44L ISG15 FAM20A | 0.872 | 0.862 | 0.882 | 0.902 | 0.835 | 0.969 | 0.940 | 0.864 | 1 |
| IFIT1 HERC6 SLC1A2 IL1R2 FAM20A MMP8 | 0.872 | 0.862 | 0.881 | 0.915 | 0.863 | 0.967 | 0.929 | 0.842 | 1 |
| IFIT1 IFI44L HERC6 SLC1A2 IL1R2 FAM20A | 0.872 | 0.862 | 0.881 | 0.913 | 0.861 | 0.965 | 0.926 | 0.836 | 1 |
| OAS1 HERC6 SLC1A2 IL1R2 OLAH FAM20A | 0.872 | 0.862 | 0.881 | 0.913 | 0.858 | 0.967 | 0.927 | 0.842 | 1 |
| RSAD2 IFI44L SLC1A2 IL1R2 OLAH FAM20A | 0.872 | 0.862 | 0.881 | 0.912 | 0.856 | 0.968 | 0.929 | 0.852 | 1 |
| RSAD2 HERC6 IL1R2 OLAH FAM20A RETN | 0.872 | 0.862 | 0.881 | 0.907 | 0.845 | 0.968 | 0.937 | 0.863 | 1 |
| IFI44L SIGLEC1 ISG15 HERC6 IL1R2 OLAH | 0.872 | 0.862 | 0.881 | 0.902 | 0.841 | 0.963 | 0.960 | 0.919 | 1 |
| OAS1 SIGLEC1 ISG15 HERC6 SLC1A2 IL1R2 | 0.872 | 0.862 | 0.881 | 0.910 | 0.857 | 0.963 | 0.965 | 0.927 | 1 |
| OAS1 SIGLEC1 HERC6 SLC1A2 IL1R2 MMP8 | 0.872 | 0.863 | 0.880 | 0.910 | 0.857 | 0.963 | 0.964 | 0.927 | 1 |
| RSAD2 SIGLEC1 ISG15 HERC6 IL1R2 MMP8 | 0.872 | 0.863 | 0.881 | 0.904 | 0.847 | 0.962 | 0.961 | 0.921 | 1 |
| SIGLEC1 ISG15 SLC1A2 IL1R2 OLAH RETN | 0.872 | 0.863 | 0.881 | 0.913 | 0.857 | 0.968 | 0.961 | 0.920 | 1 |
| RSAD2 OAS1 SIGLEC1 HERC6 IL1R2 OLAH | 0.872 | 0.862 | 0.881 | 0.907 | 0.849 | 0.965 | 0.958 | 0.915 | 1.000 |
| HERC6 SLC1A2 IL1R2 OLAH FAM20A MMP8 | 0.872 | 0.862 | 0.881 | 0.912 | 0.861 | 0.964 | 0.921 | 0.830 | 1 |
| IFI44L SIGLEC1 ISG15 HERC6 IL1R2 RETN | 0.872 | 0.862 | 0.881 | 0.901 | 0.838 | 0.965 | 0.960 | 0.919 | 1 |
| IFIT1 ISG15 HERC6 SLC1A2 IL1R2 FAM20A | 0.871 | 0.862 | 0.881 | 0.911 | 0.857 | 0.965 | 0.928 | 0.842 | 1 |
| OAS1 HERC6 IL1R2 OLAH FAM20A RETN | 0.871 | 0.861 | 0.881 | 0.906 | 0.845 | 0.967 | 0.937 | 0.863 | 1 |
| OAS1 IFIT1 HERC6 IL1R2 FAM20A RETN | 0.871 | 0.862 | 0.881 | 0.905 | 0.842 | 0.968 | 0.937 | 0.856 | 1 |
| IFI27 OAS1 IFIT1 ISG15 HERC6 FAM20A | 0.871 | 0.861 | 0.882 | 0.905 | 0.844 | 0.967 | 0.926 | 0.843 | 1 |
| IFI27 ISG15 HERC6 SLC1A2 RETN MMP8 | 0.871 | 0.862 | 0.881 | 0.907 | 0.846 | 0.969 | 0.938 | 0.870 | 1 |
| RSAD2 SIGLEC1 SLC1A2 IL1R2 OLAH RETN | 0.871 | 0.862 | 0.881 | 0.914 | 0.858 | 0.969 | 0.964 | 0.927 | 1 |
| IFIT1 IFI44L SIGLEC1 HERC6 OLAH RETN | 0.871 | 0.862 | 0.881 | 0.901 | 0.836 | 0.966 | 0.937 | 0.883 | 0.991 |
| IFI44L ISG15 IL1R2 OLAH FAM20A MMP8 | 0.871 | 0.862 | 0.880 | 0.905 | 0.844 | 0.965 | 0.948 | 0.884 | 1 |
| RSAD2 SIGLEC1 SLC1A2 FAM20A RETN MMP8 | 0.871 | 0.861 | 0.881 | 0.908 | 0.848 | 0.969 | 0.953 | 0.885 | 1 |
| RSAD2 IFIT1 HERC6 IL1R2 OLAH FAM20A | 0.871 | 0.862 | 0.881 | 0.906 | 0.845 | 0.967 | 0.937 | 0.863 | 1 |
| RSAD2 OAS1 ISG15 IL1R2 OLAH FAM20A | 0.871 | 0.862 | 0.881 | 0.900 | 0.834 | 0.966 | 0.940 | 0.866 | 1 |
| RSAD2 OAS1 SIGLEC1 ISG15 HERC6 IL1R2 | 0.871 | 0.862 | 0.880 | 0.904 | 0.846 | 0.961 | 0.959 | 0.917 | 1 |
| IFIT1 SIGLEC1 SLC1A2 OLAH RETN MMP8 | 0.871 | 0.862 | 0.881 | 0.912 | 0.856 | 0.968 | 0.959 | 0.917 | 1 |
| IFIT1 SIGLEC1 HERC6 SLC1A2 IL1R2 MMP8 | 0.871 | 0.862 | 0.880 | 0.909 | 0.856 | 0.963 | 0.964 | 0.927 | 1 |
| RSAD2 IFI44L SIGLEC1 SLC1A2 OLAH MMP8 | 0.871 | 0.862 | 0.881 | 0.911 | 0.856 | 0.967 | 0.955 | 0.913 | 0.998 |
| RSAD2 IFI44L SIGLEC1 ISG15 HERC6 FAM20A | 0.871 | 0.862 | 0.881 | 0.902 | 0.838 | 0.966 | 0.943 | 0.875 | 1 |
| RSAD2 IFI44L HERC6 SLC1A2 OLAH FAM20A | 0.871 | 0.861 | 0.882 | 0.913 | 0.858 | 0.969 | 0.924 | 0.843 | 1 |
| RSAD2 IFI27 OAS1 IFIT1 HERC6 FAM20A | 0.871 | 0.861 | 0.882 | 0.904 | 0.843 | 0.965 | 0.932 | 0.849 | 1 |
| RSAD2 SIGLEC1 ISG15 HERC6 SLC1A2 OLAH | 0.871 | 0.862 | 0.880 | 0.910 | 0.854 | 0.966 | 0.947 | 0.898 | 0.995 |
| OAS1 ISG15 IL1R2 OLAH FAM20A MMP8 | 0.871 | 0.862 | 0.881 | 0.904 | 0.841 | 0.966 | 0.937 | 0.859 | 1 |
| RSAD2 OAS1 SIGLEC1 HERC6 SLC1A2 IL1R2 | 0.871 | 0.862 | 0.881 | 0.909 | 0.855 | 0.963 | 0.966 | 0.929 | 1 |
| OAS1 SIGLEC1 HERC6 SLC1A2 OLAH RETN | 0.871 | 0.861 | 0.881 | 0.912 | 0.856 | 0.968 | 0.948 | 0.897 | 0.999 |
| OAS1 SIGLEC1 SLC1A2 OLAH RETN MMP8 | 0.871 | 0.861 | 0.881 | 0.912 | 0.854 | 0.969 | 0.960 | 0.918 | 1 |
| RSAD2 IFIT1 SIGLEC1 ISG15 HERC6 IL1R2 | 0.871 | 0.862 | 0.880 | 0.904 | 0.846 | 0.963 | 0.952 | 0.907 | 0.998 |
| IFIT1 SIGLEC1 HERC6 IL1R2 OLAH RETN | 0.871 | 0.862 | 0.880 | 0.904 | 0.842 | 0.967 | 0.949 | 0.902 | 0.996 |
| RSAD2 SIGLEC1 ISG15 FAM20A RETN MMP8 | 0.871 | 0.861 | 0.881 | 0.903 | 0.836 | 0.970 | 0.957 | 0.898 | 1 |
| RSAD2 IFI27 OAS1 SLC1A2 FAM20A RETN | 0.871 | 0.861 | 0.881 | 0.912 | 0.851 | 0.972 | 0.941 | 0.861 | 1 |
| RSAD2 SIGLEC1 HERC6 SLC1A2 IL1R2 MMP8 | 0.871 | 0.862 | 0.880 | 0.908 | 0.854 | 0.962 | 0.963 | 0.925 | 1 |
| OAS1 SIGLEC1 ISG15 SLC1A2 IL1R2 OLAH | 0.871 | 0.862 | 0.880 | 0.912 | 0.857 | 0.967 | 0.962 | 0.924 | 1 |
| OAS1 IFI44L SIGLEC1 ISG15 HERC6 IL1R2 | 0.871 | 0.862 | 0.880 | 0.904 | 0.845 | 0.962 | 0.955 | 0.911 | 1.000 |
| OAS1 SIGLEC1 ISG15 HERC6 IL1R2 MMP8 | 0.871 | 0.862 | 0.880 | 0.904 | 0.847 | 0.961 | 0.965 | 0.928 | 1 |
| IFI27 IFIT1 SLC1A2 FAM20A RETN MMP8 | 0.871 | 0.861 | 0.882 | 0.908 | 0.845 | 0.971 | 0.941 | 0.861 | 1 |
| SIGLEC1 ISG15 HERC6 SLC1A2 FAM20A RETN | 0.871 | 0.861 | 0.881 | 0.901 | 0.838 | 0.965 | 0.950 | 0.876 | 1 |
| RSAD2 IFIT1 IFI44L HERC6 IL1R2 FAM20A | 0.871 | 0.862 | 0.880 | 0.907 | 0.847 | 0.967 | 0.934 | 0.853 | 1 |
| OAS1 IFIT1 SLC1A2 IL1R2 FAM20A MMP8 | 0.871 | 0.862 | 0.880 | 0.912 | 0.858 | 0.966 | 0.934 | 0.852 | 1 |
| OAS1 SIGLEC1 SLC1A2 FAM20A RETN MMP8 | 0.871 | 0.861 | 0.881 | 0.904 | 0.842 | 0.967 | 0.950 | 0.880 | 1 |
| IFI44L SIGLEC1 HERC6 SLC1A2 OLAH MMP8 | 0.871 | 0.861 | 0.881 | 0.911 | 0.855 | 0.967 | 0.957 | 0.915 | 0.998 |
| OAS1 IFIT1 IFI44L HERC6 OLAH FAM20A | 0.871 | 0.861 | 0.881 | 0.901 | 0.837 | 0.965 | 0.937 | 0.869 | 1 |
| OAS1 IFI44L IL1R2 OLAH FAM20A MMP8 | 0.871 | 0.861 | 0.881 | 0.905 | 0.844 | 0.967 | 0.943 | 0.872 | 1 |
| IFIT1 SIGLEC1 ISG15 SLC1A2 OLAH RETN | 0.871 | 0.862 | 0.880 | 0.914 | 0.859 | 0.970 | 0.948 | 0.897 | 0.999 |
| IFIT1 IFI44L SIGLEC1 HERC6 IL1R2 OLAH | 0.871 | 0.862 | 0.880 | 0.905 | 0.846 | 0.964 | 0.952 | 0.907 | 0.997 |
| IFI44L SIGLEC1 HERC6 SLC1A2 IL1R2 OLAH | 0.871 | 0.861 | 0.881 | 0.910 | 0.855 | 0.966 | 0.960 | 0.919 | 1 |
| IFIT1 ISG15 HERC6 OLAH FAM20A RETN | 0.871 | 0.861 | 0.881 | 0.901 | 0.838 | 0.965 | 0.943 | 0.877 | 1 |
| IFI27 IFI44L SIGLEC1 ISG15 HERC6 MMP8 | 0.871 | 0.862 | 0.880 | 0.897 | 0.835 | 0.959 | 0.910 | 0.836 | 0.983 |
| RSAD2 IFI27 IFI44L SIGLEC1 HERC6 MMP8 | 0.871 | 0.862 | 0.880 | 0.901 | 0.843 | 0.959 | 0.907 | 0.831 | 0.982 |
| IFIT1 SIGLEC1 SLC1A2 IL1R2 OLAH RETN | 0.871 | 0.862 | 0.880 | 0.914 | 0.858 | 0.969 | 0.958 | 0.914 | 1 |
| ISG15 HERC6 SLC1A2 IL1R2 FAM20A RETN | 0.871 | 0.861 | 0.881 | 0.912 | 0.858 | 0.966 | 0.926 | 0.837 | 1 |
| RSAD2 IFI44L SIGLEC1 ISG15 HERC6 IL1R2 | 0.871 | 0.862 | 0.880 | 0.900 | 0.838 | 0.962 | 0.958 | 0.916 | 0.999 |
| SIGLEC1 ISG15 HERC6 IL1R2 RETN MMP8 | 0.871 | 0.861 | 0.880 | 0.901 | 0.836 | 0.966 | 0.970 | 0.937 | 1 |
| RSAD2 IFI44L SIGLEC1 HERC6 FAM20A RETN | 0.871 | 0.861 | 0.880 | 0.907 | 0.844 | 0.970 | 0.943 | 0.876 | 1 |
| IFIT1 HERC6 SLC1A2 IL1R2 OLAH FAM20A | 0.871 | 0.861 | 0.881 | 0.912 | 0.857 | 0.967 | 0.927 | 0.843 | 1 |
| RSAD2 IFI44L SIGLEC1 HERC6 SLC1A2 FAM20A | 0.871 | 0.861 | 0.880 | 0.909 | 0.852 | 0.966 | 0.939 | 0.863 | 1 |
| RSAD2 IFI44L SIGLEC1 ISG15 OLAH FAM20A | 0.871 | 0.861 | 0.881 | 0.897 | 0.826 | 0.968 | 0.963 | 0.921 | 1 |
| IFI44L SIGLEC1 ISG15 HERC6 SLC1A2 OLAH | 0.871 | 0.861 | 0.880 | 0.910 | 0.854 | 0.966 | 0.950 | 0.903 | 0.997 |
| OAS1 SIGLEC1 ISG15 HERC6 SLC1A2 FAM20A | 0.871 | 0.861 | 0.881 | 0.908 | 0.851 | 0.965 | 0.943 | 0.871 | 1 |
| OAS1 IFI44L HERC6 IL1R2 FAM20A RETN | 0.871 | 0.861 | 0.880 | 0.907 | 0.845 | 0.968 | 0.937 | 0.857 | 1 |
| RSAD2 IFIT1 SIGLEC1 HERC6 IL1R2 OLAH | 0.871 | 0.861 | 0.880 | 0.907 | 0.849 | 0.966 | 0.952 | 0.907 | 0.997 |
| RSAD2 OAS1 SLC1A2 IL1R2 OLAH FAM20A | 0.871 | 0.861 | 0.880 | 0.912 | 0.857 | 0.968 | 0.933 | 0.850 | 1 |
| SIGLEC1 ISG15 SLC1A2 OLAH RETN MMP8 | 0.871 | 0.861 | 0.880 | 0.910 | 0.852 | 0.968 | 0.959 | 0.917 | 1.000 |
| OAS1 ISG15 HERC6 SLC1A2 OLAH FAM20A | 0.871 | 0.860 | 0.881 | 0.912 | 0.856 | 0.967 | 0.929 | 0.849 | 1 |
| IFIT1 SIGLEC1 ISG15 HERC6 SLC1A2 IL1R2 | 0.871 | 0.862 | 0.879 | 0.909 | 0.855 | 0.963 | 0.962 | 0.923 | 1 |
| RSAD2 SIGLEC1 HERC6 SLC1A2 IL1R2 RETN | 0.871 | 0.861 | 0.880 | 0.910 | 0.854 | 0.965 | 0.958 | 0.916 | 1.000 |
| RSAD2 IFI27 IFIT1 SLC1A2 FAM20A RETN | 0.871 | 0.861 | 0.881 | 0.908 | 0.847 | 0.969 | 0.940 | 0.859 | 1 |
| IFIT1 IFI44L IL1R2 OLAH FAM20A MMP8 | 0.871 | 0.861 | 0.880 | 0.906 | 0.845 | 0.967 | 0.950 | 0.888 | 1 |
| SIGLEC1 HERC6 IL1R2 OLAH RETN MMP8 | 0.871 | 0.861 | 0.881 | 0.901 | 0.836 | 0.965 | 0.968 | 0.935 | 1 |
| SIGLEC1 ISG15 HERC6 IL1R2 OLAH MMP8 | 0.871 | 0.861 | 0.880 | 0.904 | 0.845 | 0.963 | 0.964 | 0.927 | 1 |
| RSAD2 IFI44L HERC6 SLC1A2 IL1R2 FAM20A | 0.871 | 0.861 | 0.880 | 0.913 | 0.860 | 0.965 | 0.924 | 0.834 | 1 |
| OAS1 SIGLEC1 SLC1A2 IL1R2 OLAH RETN | 0.871 | 0.861 | 0.880 | 0.913 | 0.857 | 0.968 | 0.962 | 0.922 | 1 |
| IFI44L SIGLEC1 ISG15 HERC6 SLC1A2 FAM20A | 0.871 | 0.861 | 0.880 | 0.902 | 0.840 | 0.964 | 0.952 | 0.879 | 1 |
| IFI44L ISG15 SLC1A2 IL1R2 FAM20A RETN | 0.871 | 0.861 | 0.880 | 0.908 | 0.854 | 0.963 | 0.928 | 0.842 | 1 |
| IFI44L SLC1A2 IL1R2 FAM20A RETN MMP8 | 0.871 | 0.862 | 0.879 | 0.913 | 0.861 | 0.965 | 0.927 | 0.836 | 1 |
| RSAD2 OAS1 IFIT1 IL1R2 OLAH FAM20A | 0.871 | 0.861 | 0.880 | 0.901 | 0.835 | 0.968 | 0.945 | 0.873 | 1 |
| SIGLEC1 HERC6 SLC1A2 OLAH RETN MMP8 | 0.871 | 0.860 | 0.881 | 0.909 | 0.850 | 0.968 | 0.964 | 0.927 | 1 |
| OAS1 HERC6 SLC1A2 IL1R2 FAM20A MMP8 | 0.871 | 0.861 | 0.880 | 0.914 | 0.862 | 0.966 | 0.924 | 0.831 | 1 |
| RSAD2 OAS1 HERC6 SLC1A2 IL1R2 FAM20A | 0.870 | 0.861 | 0.880 | 0.910 | 0.856 | 0.964 | 0.927 | 0.837 | 1 |
| RSAD2 HERC6 IL1R2 FAM20A RETN MMP8 | 0.870 | 0.861 | 0.880 | 0.906 | 0.844 | 0.968 | 0.937 | 0.854 | 1 |
| RSAD2 IFI27 ISG15 SLC1A2 FAM20A RETN | 0.870 | 0.860 | 0.881 | 0.909 | 0.848 | 0.971 | 0.936 | 0.854 | 1 |
| IFIT1 IFI44L SLC1A2 OLAH FAM20A RETN | 0.870 | 0.861 | 0.880 | 0.910 | 0.851 | 0.968 | 0.918 | 0.841 | 0.996 |
| RSAD2 OAS1 IFI44L IL1R2 OLAH FAM20A | 0.870 | 0.861 | 0.880 | 0.901 | 0.835 | 0.966 | 0.946 | 0.878 | 1 |
| OAS1 IFI44L HERC6 SLC1A2 IL1R2 FAM20A | 0.870 | 0.861 | 0.880 | 0.912 | 0.860 | 0.965 | 0.926 | 0.836 | 1 |
| RSAD2 SIGLEC1 ISG15 HERC6 IL1R2 RETN | 0.870 | 0.861 | 0.880 | 0.904 | 0.842 | 0.965 | 0.955 | 0.912 | 0.999 |
| RSAD2 OAS1 HERC6 SLC1A2 OLAH FAM20A | 0.870 | 0.860 | 0.880 | 0.910 | 0.854 | 0.966 | 0.930 | 0.850 | 1 |
| OAS1 IFI44L SIGLEC1 HERC6 IL1R2 OLAH | 0.870 | 0.861 | 0.880 | 0.904 | 0.844 | 0.964 | 0.958 | 0.916 | 1.000 |
| OAS1 IFIT1 SIGLEC1 HERC6 SLC1A2 RETN | 0.870 | 0.861 | 0.880 | 0.906 | 0.847 | 0.965 | 0.927 | 0.867 | 0.987 |
| IFI27 IFIT1 IFI44L ISG15 FAM20A MMP8 | 0.870 | 0.860 | 0.881 | 0.900 | 0.833 | 0.966 | 0.933 | 0.853 | 1 |
| HERC6 SLC1A2 IL1R2 OLAH FAM20A RETN | 0.870 | 0.860 | 0.880 | 0.913 | 0.859 | 0.967 | 0.922 | 0.834 | 1 |
| RSAD2 IFIT1 IFI44L SIGLEC1 SLC1A2 OLAH | 0.870 | 0.861 | 0.879 | 0.913 | 0.856 | 0.969 | 0.947 | 0.897 | 0.996 |
| RSAD2 SIGLEC1 ISG15 HERC6 SLC1A2 IL1R2 | 0.870 | 0.862 | 0.879 | 0.909 | 0.855 | 0.964 | 0.960 | 0.919 | 1 |
| IFIT1 HERC6 SLC1A2 OLAH FAM20A MMP8 | 0.870 | 0.860 | 0.881 | 0.910 | 0.854 | 0.966 | 0.932 | 0.853 | 1 |
| RSAD2 IFI27 HERC6 SLC1A2 FAM20A MMP8 | 0.870 | 0.860 | 0.880 | 0.911 | 0.855 | 0.967 | 0.928 | 0.845 | 1 |
| RSAD2 SIGLEC1 HERC6 SLC1A2 OLAH RETN | 0.870 | 0.860 | 0.880 | 0.913 | 0.857 | 0.970 | 0.946 | 0.896 | 0.995 |
| IFI44L SIGLEC1 ISG15 SLC1A2 FAM20A RETN | 0.870 | 0.861 | 0.880 | 0.901 | 0.837 | 0.965 | 0.948 | 0.875 | 1 |
| OAS1 IFIT1 ISG15 IL1R2 FAM20A MMP8 | 0.870 | 0.861 | 0.880 | 0.905 | 0.843 | 0.968 | 0.943 | 0.870 | 1 |
| RSAD2 IFIT1 SIGLEC1 ISG15 SLC1A2 OLAH | 0.870 | 0.861 | 0.879 | 0.912 | 0.855 | 0.968 | 0.946 | 0.895 | 0.996 |
| RSAD2 OAS1 IFI44L IL1R2 FAM20A MMP8 | 0.870 | 0.861 | 0.880 | 0.905 | 0.845 | 0.966 | 0.937 | 0.859 | 1 |
| IFI44L SIGLEC1 ISG15 SLC1A2 IL1R2 MMP8 | 0.870 | 0.862 | 0.878 | 0.908 | 0.854 | 0.962 | 0.966 | 0.930 | 1 |
| IFI27 IFIT1 HERC6 SLC1A2 FAM20A MMP8 | 0.870 | 0.860 | 0.880 | 0.911 | 0.855 | 0.967 | 0.927 | 0.843 | 1 |
| RSAD2 SIGLEC1 SLC1A2 OLAH RETN MMP8 | 0.870 | 0.861 | 0.880 | 0.910 | 0.853 | 0.968 | 0.960 | 0.919 | 1 |
| IFI44L SIGLEC1 ISG15 IL1R2 OLAH MMP8 | 0.870 | 0.862 | 0.879 | 0.905 | 0.848 | 0.962 | 0.957 | 0.914 | 0.999 |
| RSAD2 IFIT1 SLC1A2 IL1R2 FAM20A MMP8 | 0.870 | 0.861 | 0.879 | 0.910 | 0.855 | 0.965 | 0.932 | 0.849 | 1 |
| OAS1 IFIT1 ISG15 IL1R2 OLAH FAM20A | 0.870 | 0.861 | 0.880 | 0.901 | 0.835 | 0.967 | 0.945 | 0.873 | 1 |
| OAS1 SIGLEC1 ISG15 FAM20A RETN MMP8 | 0.870 | 0.860 | 0.881 | 0.901 | 0.832 | 0.971 | 0.958 | 0.898 | 1 |
| OAS1 SIGLEC1 HERC6 SLC1A2 FAM20A MMP8 | 0.870 | 0.860 | 0.880 | 0.908 | 0.851 | 0.965 | 0.943 | 0.870 | 1 |
| OAS1 IFI44L SIGLEC1 HERC6 SLC1A2 OLAH | 0.870 | 0.860 | 0.880 | 0.912 | 0.856 | 0.967 | 0.951 | 0.904 | 0.998 |
| OAS1 IFI44L SLC1A2 IL1R2 OLAH FAM20A | 0.870 | 0.860 | 0.880 | 0.913 | 0.857 | 0.969 | 0.930 | 0.852 | 1 |
| RSAD2 IFI44L SIGLEC1 HERC6 IL1R2 RETN | 0.870 | 0.861 | 0.879 | 0.901 | 0.837 | 0.965 | 0.960 | 0.920 | 1 |
| RSAD2 OAS1 SIGLEC1 SLC1A2 FAM20A MMP8 | 0.870 | 0.860 | 0.880 | 0.903 | 0.840 | 0.966 | 0.946 | 0.873 | 1 |
| RSAD2 OAS1 SIGLEC1 ISG15 SLC1A2 OLAH | 0.870 | 0.860 | 0.880 | 0.910 | 0.854 | 0.967 | 0.948 | 0.898 | 0.998 |
| IFI27 OAS1 HERC6 SLC1A2 FAM20A MMP8 | 0.870 | 0.860 | 0.880 | 0.915 | 0.860 | 0.969 | 0.925 | 0.840 | 1 |
| RSAD2 ISG15 HERC6 SLC1A2 OLAH FAM20A | 0.870 | 0.860 | 0.880 | 0.911 | 0.855 | 0.967 | 0.932 | 0.852 | 1 |
| IFIT1 IFI44L SIGLEC1 SLC1A2 IL1R2 OLAH | 0.870 | 0.861 | 0.879 | 0.914 | 0.859 | 0.968 | 0.963 | 0.925 | 1 |
| RSAD2 OAS1 SIGLEC1 ISG15 FAM20A RETN | 0.870 | 0.860 | 0.880 | 0.896 | 0.826 | 0.967 | 0.954 | 0.895 | 1 |
| OAS1 IFIT1 HERC6 SLC1A2 IL1R2 FAM20A | 0.870 | 0.860 | 0.880 | 0.913 | 0.859 | 0.966 | 0.932 | 0.848 | 1 |
| OAS1 HERC6 SLC1A2 OLAH FAM20A MMP8 | 0.870 | 0.860 | 0.880 | 0.912 | 0.857 | 0.967 | 0.932 | 0.852 | 1 |
| RSAD2 IFIT1 SIGLEC1 HERC6 SLC1A2 IL1R2 | 0.870 | 0.861 | 0.879 | 0.908 | 0.853 | 0.963 | 0.963 | 0.925 | 1 |
| OAS1 IFIT1 SIGLEC1 IL1R2 OLAH MMP8 | 0.870 | 0.861 | 0.879 | 0.905 | 0.847 | 0.964 | 0.960 | 0.920 | 0.999 |
| OAS1 IFIT1 IFI44L IL1R2 FAM20A MMP8 | 0.870 | 0.861 | 0.879 | 0.904 | 0.841 | 0.967 | 0.943 | 0.868 | 1 |
| IFI44L SIGLEC1 ISG15 SLC1A2 IL1R2 RETN | 0.870 | 0.862 | 0.878 | 0.910 | 0.855 | 0.965 | 0.965 | 0.927 | 1 |
| RSAD2 SIGLEC1 ISG15 HERC6 IL1R2 OLAH | 0.870 | 0.861 | 0.879 | 0.905 | 0.845 | 0.964 | 0.958 | 0.916 | 0.999 |
| RSAD2 HERC6 SLC1A2 OLAH FAM20A MMP8 | 0.870 | 0.860 | 0.880 | 0.912 | 0.857 | 0.967 | 0.933 | 0.853 | 1 |
| IFIT1 ISG15 IL1R2 OLAH FAM20A MMP8 | 0.870 | 0.860 | 0.880 | 0.906 | 0.845 | 0.967 | 0.949 | 0.883 | 1 |
| OAS1 IFI44L SIGLEC1 OLAH FAM20A RETN | 0.870 | 0.860 | 0.880 | 0.896 | 0.823 | 0.968 | 0.964 | 0.921 | 1 |
| RSAD2 IFIT1 IL1R2 OLAH FAM20A MMP8 | 0.870 | 0.861 | 0.879 | 0.905 | 0.843 | 0.967 | 0.946 | 0.876 | 1 |
| RSAD2 OAS1 IFI44L SIGLEC1 HERC6 FAM20A | 0.870 | 0.860 | 0.879 | 0.899 | 0.833 | 0.964 | 0.941 | 0.871 | 1 |
| IFIT1 IFI44L ISG15 SLC1A2 IL1R2 FAM20A | 0.870 | 0.861 | 0.879 | 0.908 | 0.853 | 0.964 | 0.924 | 0.836 | 1 |
| RSAD2 IFI44L SIGLEC1 HERC6 IL1R2 OLAH | 0.870 | 0.860 | 0.879 | 0.902 | 0.841 | 0.963 | 0.959 | 0.918 | 1.000 |
| IFIT1 IFI44L ISG15 HERC6 FAM20A RETN | 0.870 | 0.860 | 0.879 | 0.904 | 0.840 | 0.968 | 0.935 | 0.855 | 1 |
| IFI44L SIGLEC1 HERC6 IL1R2 OLAH RETN | 0.870 | 0.860 | 0.879 | 0.900 | 0.836 | 0.965 | 0.958 | 0.916 | 0.999 |
| OAS1 IFIT1 IFI44L SIGLEC1 IL1R2 MMP8 | 0.870 | 0.861 | 0.879 | 0.900 | 0.840 | 0.961 | 0.958 | 0.916 | 1.000 |
| ISG15 HERC6 SLC1A2 IL1R2 FAM20A MMP8 | 0.870 | 0.860 | 0.880 | 0.915 | 0.864 | 0.966 | 0.921 | 0.824 | 1 |
| IFI27 IFI44L HERC6 SLC1A2 FAM20A MMP8 | 0.870 | 0.859 | 0.880 | 0.906 | 0.846 | 0.966 | 0.930 | 0.847 | 1 |
| SIGLEC1 ISG15 HERC6 SLC1A2 IL1R2 MMP8 | 0.870 | 0.861 | 0.878 | 0.907 | 0.852 | 0.962 | 0.963 | 0.925 | 1 |
| RSAD2 IFIT1 SIGLEC1 SLC1A2 OLAH RETN | 0.870 | 0.860 | 0.879 | 0.914 | 0.858 | 0.970 | 0.952 | 0.905 | 0.999 |
| RSAD2 OAS1 SIGLEC1 SLC1A2 OLAH RETN | 0.870 | 0.860 | 0.879 | 0.912 | 0.856 | 0.969 | 0.958 | 0.910 | 1 |
| RSAD2 IFIT1 HERC6 IL1R2 FAM20A RETN | 0.870 | 0.860 | 0.879 | 0.904 | 0.841 | 0.966 | 0.938 | 0.857 | 1 |
| OAS1 IFI44L SIGLEC1 SLC1A2 IL1R2 OLAH | 0.870 | 0.860 | 0.879 | 0.913 | 0.858 | 0.968 | 0.963 | 0.925 | 1 |
| OAS1 IFI44L SIGLEC1 SLC1A2 OLAH RETN | 0.870 | 0.860 | 0.880 | 0.914 | 0.858 | 0.970 | 0.950 | 0.900 | 1.000 |
| RSAD2 IFI27 SIGLEC1 ISG15 HERC6 MMP8 | 0.870 | 0.861 | 0.879 | 0.902 | 0.844 | 0.959 | 0.909 | 0.834 | 0.983 |
| SIGLEC1 HERC6 SLC1A2 IL1R2 OLAH RETN | 0.870 | 0.860 | 0.880 | 0.914 | 0.860 | 0.969 | 0.959 | 0.917 | 1 |
| IFI27 OAS1 IFIT1 IFI44L ISG15 RETN | 0.870 | 0.860 | 0.880 | 0.906 | 0.843 | 0.969 | 0.952 | 0.890 | 1 |
| OAS1 IFI44L SIGLEC1 ISG15 SLC1A2 OLAH | 0.870 | 0.860 | 0.879 | 0.913 | 0.857 | 0.968 | 0.947 | 0.897 | 0.997 |
| RSAD2 OAS1 SIGLEC1 ISG15 SLC1A2 FAM20A | 0.870 | 0.860 | 0.880 | 0.902 | 0.839 | 0.965 | 0.947 | 0.874 | 1 |
| IFI44L SIGLEC1 HERC6 SLC1A2 IL1R2 MMP8 | 0.870 | 0.861 | 0.879 | 0.907 | 0.853 | 0.962 | 0.964 | 0.927 | 1 |
| RSAD2 IFI44L SIGLEC1 SLC1A2 IL1R2 OLAH | 0.870 | 0.860 | 0.879 | 0.912 | 0.857 | 0.968 | 0.965 | 0.929 | 1 |
| RSAD2 IFI44L SIGLEC1 HERC6 SLC1A2 OLAH | 0.870 | 0.860 | 0.879 | 0.912 | 0.857 | 0.968 | 0.947 | 0.898 | 0.995 |
| RSAD2 IFI27 IFI44L ISG15 FAM20A MMP8 | 0.870 | 0.859 | 0.880 | 0.899 | 0.832 | 0.965 | 0.937 | 0.860 | 1 |
| IFI44L HERC6 SLC1A2 OLAH FAM20A RETN | 0.870 | 0.859 | 0.880 | 0.913 | 0.857 | 0.970 | 0.934 | 0.856 | 1 |
| IFI27 OAS1 IFIT1 SIGLEC1 SLC1A2 MMP8 | 0.870 | 0.861 | 0.879 | 0.904 | 0.846 | 0.963 | 0.929 | 0.861 | 0.997 |
| IFI44L SIGLEC1 ISG15 SLC1A2 FAM20A MMP8 | 0.870 | 0.860 | 0.879 | 0.901 | 0.838 | 0.965 | 0.951 | 0.879 | 1 |
| OAS1 IFIT1 SIGLEC1 HERC6 OLAH MMP8 | 0.870 | 0.860 | 0.879 | 0.900 | 0.839 | 0.961 | 0.939 | 0.886 | 0.992 |
| RSAD2 HERC6 SLC1A2 IL1R2 FAM20A MMP8 | 0.870 | 0.860 | 0.879 | 0.914 | 0.862 | 0.965 | 0.925 | 0.832 | 1 |
| IFI44L HERC6 SLC1A2 IL1R2 FAM20A RETN | 0.870 | 0.860 | 0.880 | 0.913 | 0.860 | 0.966 | 0.925 | 0.836 | 1 |
| IFI44L HERC6 OLAH FAM20A RETN MMP8 | 0.870 | 0.859 | 0.880 | 0.902 | 0.836 | 0.969 | 0.948 | 0.884 | 1 |
| IFIT1 HERC6 OLAH FAM20A RETN MMP8 | 0.869 | 0.859 | 0.880 | 0.901 | 0.836 | 0.965 | 0.948 | 0.882 | 1 |
| IFIT1 ISG15 SLC1A2 IL1R2 OLAH FAM20A | 0.869 | 0.860 | 0.879 | 0.907 | 0.849 | 0.965 | 0.933 | 0.858 | 1 |
| OAS1 ISG15 SLC1A2 IL1R2 OLAH FAM20A | 0.869 | 0.860 | 0.879 | 0.911 | 0.854 | 0.968 | 0.927 | 0.844 | 1 |
| OAS1 SIGLEC1 ISG15 OLAH FAM20A RETN | 0.869 | 0.859 | 0.880 | 0.896 | 0.825 | 0.968 | 0.962 | 0.918 | 1 |
| OAS1 IFI44L ISG15 SLC1A2 OLAH FAM20A | 0.869 | 0.859 | 0.880 | 0.909 | 0.849 | 0.968 | 0.927 | 0.852 | 1 |
| IFI27 IFIT1 IFI44L HERC6 SLC1A2 FAM20A | 0.869 | 0.859 | 0.880 | 0.905 | 0.846 | 0.964 | 0.930 | 0.847 | 1 |
| RSAD2 ISG15 SLC1A2 IL1R2 OLAH FAM20A | 0.869 | 0.860 | 0.879 | 0.912 | 0.856 | 0.968 | 0.932 | 0.851 | 1 |
| RSAD2 IFI27 SIGLEC1 HERC6 SLC1A2 MMP8 | 0.869 | 0.860 | 0.878 | 0.903 | 0.846 | 0.959 | 0.926 | 0.859 | 0.993 |
| OAS1 IFIT1 SLC1A2 IL1R2 OLAH FAM20A | 0.869 | 0.860 | 0.879 | 0.911 | 0.853 | 0.968 | 0.935 | 0.856 | 1 |
| IFI27 OAS1 SLC1A2 FAM20A RETN MMP8 | 0.869 | 0.859 | 0.880 | 0.911 | 0.849 | 0.972 | 0.940 | 0.858 | 1 |
| OAS1 IFI44L SIGLEC1 HERC6 SLC1A2 FAM20A | 0.869 | 0.860 | 0.879 | 0.907 | 0.850 | 0.964 | 0.943 | 0.870 | 1 |
| RSAD2 IFI44L ISG15 SLC1A2 OLAH FAM20A | 0.869 | 0.860 | 0.879 | 0.908 | 0.849 | 0.968 | 0.926 | 0.852 | 1 |
| IFIT1 IFI44L SIGLEC1 HERC6 OLAH MMP8 | 0.869 | 0.860 | 0.879 | 0.899 | 0.837 | 0.961 | 0.937 | 0.882 | 0.992 |
| ISG15 HERC6 OLAH FAM20A RETN MMP8 | 0.869 | 0.859 | 0.880 | 0.903 | 0.838 | 0.968 | 0.951 | 0.888 | 1 |
| OAS1 IFIT1 SIGLEC1 SLC1A2 IL1R2 RETN | 0.869 | 0.860 | 0.879 | 0.910 | 0.855 | 0.965 | 0.962 | 0.922 | 1 |
| SIGLEC1 ISG15 HERC6 SLC1A2 OLAH RETN | 0.869 | 0.859 | 0.879 | 0.909 | 0.850 | 0.967 | 0.954 | 0.910 | 0.999 |
| RSAD2 IFI27 OAS1 IFIT1 ISG15 RETN | 0.869 | 0.860 | 0.879 | 0.904 | 0.841 | 0.967 | 0.950 | 0.887 | 1 |
| OAS1 IFIT1 SIGLEC1 HERC6 RETN MMP8 | 0.869 | 0.860 | 0.879 | 0.898 | 0.831 | 0.965 | 0.924 | 0.863 | 0.984 |
| SIGLEC1 HERC6 SLC1A2 IL1R2 RETN MMP8 | 0.869 | 0.860 | 0.879 | 0.909 | 0.854 | 0.965 | 0.971 | 0.939 | 1 |
| RSAD2 IFI27 SLC1A2 FAM20A RETN MMP8 | 0.869 | 0.859 | 0.879 | 0.910 | 0.848 | 0.971 | 0.938 | 0.857 | 1 |
| IFI44L SIGLEC1 SLC1A2 IL1R2 RETN MMP8 | 0.869 | 0.861 | 0.878 | 0.912 | 0.858 | 0.966 | 0.966 | 0.930 | 1 |
| OAS1 SIGLEC1 ISG15 HERC6 IL1R2 OLAH | 0.869 | 0.860 | 0.879 | 0.906 | 0.848 | 0.964 | 0.963 | 0.924 | 1 |
| RSAD2 IFIT1 SIGLEC1 HERC6 OLAH RETN | 0.869 | 0.860 | 0.879 | 0.900 | 0.835 | 0.965 | 0.938 | 0.885 | 0.991 |
| IFI44L SIGLEC1 HERC6 SLC1A2 IL1R2 RETN | 0.869 | 0.860 | 0.878 | 0.908 | 0.852 | 0.964 | 0.955 | 0.912 | 0.999 |
| IFIT1 ISG15 SLC1A2 IL1R2 FAM20A MMP8 | 0.869 | 0.860 | 0.878 | 0.909 | 0.854 | 0.965 | 0.935 | 0.855 | 1 |
| RSAD2 SIGLEC1 ISG15 SLC1A2 FAM20A MMP8 | 0.869 | 0.859 | 0.879 | 0.902 | 0.839 | 0.964 | 0.949 | 0.878 | 1 |
| IFIT1 SIGLEC1 ISG15 HERC6 OLAH MMP8 | 0.869 | 0.860 | 0.879 | 0.899 | 0.837 | 0.962 | 0.943 | 0.893 | 0.994 |
| RSAD2 IFI27 OAS1 IFI44L FAM20A MMP8 | 0.869 | 0.859 | 0.880 | 0.900 | 0.833 | 0.966 | 0.934 | 0.855 | 1 |
| RSAD2 SIGLEC1 ISG15 SLC1A2 OLAH RETN | 0.869 | 0.860 | 0.879 | 0.912 | 0.856 | 0.968 | 0.951 | 0.903 | 0.999 |
| RSAD2 SIGLEC1 ISG15 OLAH FAM20A RETN | 0.869 | 0.859 | 0.879 | 0.897 | 0.826 | 0.969 | 0.961 | 0.917 | 1 |
| IFI44L SIGLEC1 HERC6 SLC1A2 FAM20A MMP8 | 0.869 | 0.859 | 0.879 | 0.904 | 0.842 | 0.966 | 0.953 | 0.880 | 1 |
| ISG15 HERC6 SLC1A2 OLAH FAM20A RETN | 0.869 | 0.859 | 0.879 | 0.911 | 0.855 | 0.968 | 0.932 | 0.851 | 1 |
| RSAD2 IFIT1 IFI44L SLC1A2 OLAH FAM20A | 0.869 | 0.860 | 0.878 | 0.909 | 0.850 | 0.968 | 0.922 | 0.845 | 0.998 |
| IFIT1 SIGLEC1 ISG15 SLC1A2 IL1R2 RETN | 0.869 | 0.861 | 0.878 | 0.909 | 0.854 | 0.964 | 0.965 | 0.927 | 1 |
| RSAD2 OAS1 SIGLEC1 IL1R2 OLAH MMP8 | 0.869 | 0.859 | 0.879 | 0.904 | 0.845 | 0.963 | 0.962 | 0.924 | 1 |
| IFIT1 IFI44L SIGLEC1 HERC6 SLC1A2 IL1R2 | 0.869 | 0.860 | 0.878 | 0.908 | 0.853 | 0.963 | 0.959 | 0.918 | 1.000 |
| IFIT1 ISG15 HERC6 SLC1A2 OLAH FAM20A | 0.869 | 0.859 | 0.879 | 0.911 | 0.854 | 0.967 | 0.932 | 0.852 | 1 |
| HERC6 SLC1A2 OLAH FAM20A RETN MMP8 | 0.869 | 0.859 | 0.879 | 0.911 | 0.854 | 0.967 | 0.934 | 0.855 | 1 |
| IFIT1 IFI44L SIGLEC1 IL1R2 OLAH MMP8 | 0.869 | 0.860 | 0.878 | 0.904 | 0.847 | 0.962 | 0.959 | 0.917 | 1 |
| OAS1 SIGLEC1 HERC6 IL1R2 OLAH RETN | 0.869 | 0.859 | 0.879 | 0.903 | 0.840 | 0.966 | 0.955 | 0.911 | 1.000 |
| OAS1 IFI44L ISG15 IL1R2 OLAH FAM20A | 0.869 | 0.859 | 0.879 | 0.901 | 0.836 | 0.967 | 0.948 | 0.880 | 1 |
| OAS1 IFIT1 HERC6 SLC1A2 OLAH FAM20A | 0.869 | 0.859 | 0.879 | 0.910 | 0.854 | 0.966 | 0.930 | 0.849 | 1 |
| RSAD2 OAS1 IFI44L SIGLEC1 SLC1A2 OLAH | 0.869 | 0.859 | 0.879 | 0.912 | 0.856 | 0.968 | 0.951 | 0.903 | 1.000 |
| OAS1 IFIT1 SIGLEC1 SLC1A2 IL1R2 MMP8 | 0.869 | 0.860 | 0.878 | 0.910 | 0.856 | 0.964 | 0.967 | 0.932 | 1 |
| OAS1 IFIT1 SIGLEC1 IL1R2 RETN MMP8 | 0.869 | 0.859 | 0.879 | 0.900 | 0.836 | 0.964 | 0.965 | 0.929 | 1 |
| SIGLEC1 ISG15 HERC6 SLC1A2 FAM20A MMP8 | 0.869 | 0.859 | 0.879 | 0.900 | 0.838 | 0.963 | 0.952 | 0.879 | 1 |
| OAS1 IFIT1 IFI44L SLC1A2 OLAH FAM20A | 0.869 | 0.859 | 0.879 | 0.910 | 0.852 | 0.968 | 0.923 | 0.845 | 1 |
| RSAD2 OAS1 IL1R2 OLAH FAM20A RETN | 0.869 | 0.859 | 0.879 | 0.900 | 0.834 | 0.965 | 0.930 | 0.852 | 1 |
| RSAD2 IFIT1 IFI44L HERC6 OLAH FAM20A | 0.869 | 0.859 | 0.879 | 0.903 | 0.840 | 0.965 | 0.934 | 0.864 | 1 |
| IFI44L SIGLEC1 IL1R2 OLAH RETN MMP8 | 0.869 | 0.859 | 0.878 | 0.902 | 0.840 | 0.964 | 0.963 | 0.925 | 1 |
| ISG15 HERC6 SLC1A2 OLAH FAM20A MMP8 | 0.869 | 0.859 | 0.879 | 0.912 | 0.857 | 0.966 | 0.929 | 0.847 | 1 |
| RSAD2 OAS1 SLC1A2 OLAH FAM20A MMP8 | 0.869 | 0.859 | 0.878 | 0.909 | 0.852 | 0.966 | 0.934 | 0.858 | 1 |
| RSAD2 IFI44L SIGLEC1 SLC1A2 FAM20A MMP8 | 0.869 | 0.859 | 0.879 | 0.903 | 0.841 | 0.966 | 0.945 | 0.873 | 1 |
| IFI44L ISG15 HERC6 OLAH FAM20A RETN | 0.869 | 0.859 | 0.879 | 0.900 | 0.833 | 0.967 | 0.948 | 0.884 | 1 |
| OAS1 SIGLEC1 ISG15 SLC1A2 OLAH RETN | 0.869 | 0.859 | 0.879 | 0.914 | 0.858 | 0.969 | 0.948 | 0.897 | 0.999 |
| IFI27 OAS1 IFIT1 ISG15 HERC6 MMP8 | 0.869 | 0.860 | 0.877 | 0.900 | 0.843 | 0.957 | 0.900 | 0.823 | 0.977 |
| IFIT1 IFI44L SIGLEC1 ISG15 HERC6 RETN | 0.869 | 0.859 | 0.878 | 0.896 | 0.828 | 0.963 | 0.933 | 0.877 | 0.988 |
| OAS1 IFIT1 IL1R2 FAM20A RETN MMP8 | 0.869 | 0.859 | 0.878 | 0.905 | 0.842 | 0.969 | 0.948 | 0.874 | 1 |
| OAS1 IL1R2 OLAH FAM20A RETN MMP8 | 0.869 | 0.859 | 0.879 | 0.907 | 0.845 | 0.969 | 0.940 | 0.863 | 1 |
| OAS1 IFIT1 IFI44L SIGLEC1 IL1R2 OLAH | 0.869 | 0.860 | 0.878 | 0.901 | 0.837 | 0.965 | 0.954 | 0.911 | 0.998 |
| RSAD2 IFI44L SLC1A2 IL1R2 FAM20A MMP8 | 0.869 | 0.860 | 0.878 | 0.911 | 0.859 | 0.963 | 0.927 | 0.838 | 1 |
| RSAD2 IFI44L HERC6 IL1R2 FAM20A RETN | 0.869 | 0.859 | 0.879 | 0.907 | 0.846 | 0.968 | 0.934 | 0.853 | 1 |
| OAS1 IFI44L SIGLEC1 FAM20A RETN MMP8 | 0.869 | 0.859 | 0.879 | 0.902 | 0.833 | 0.971 | 0.959 | 0.899 | 1 |
| OAS1 IFI44L SIGLEC1 IL1R2 OLAH MMP8 | 0.869 | 0.860 | 0.878 | 0.903 | 0.843 | 0.963 | 0.957 | 0.914 | 0.999 |
| IFI44L SIGLEC1 HERC6 SLC1A2 OLAH RETN | 0.869 | 0.859 | 0.879 | 0.909 | 0.851 | 0.968 | 0.957 | 0.913 | 1 |
| IFIT1 SIGLEC1 SLC1A2 IL1R2 RETN MMP8 | 0.869 | 0.860 | 0.878 | 0.911 | 0.857 | 0.966 | 0.970 | 0.937 | 1 |
| OAS1 IFI44L SIGLEC1 HERC6 SLC1A2 IL1R2 | 0.869 | 0.859 | 0.878 | 0.910 | 0.856 | 0.963 | 0.959 | 0.918 | 1.000 |
| IFIT1 SIGLEC1 ISG15 SLC1A2 IL1R2 MMP8 | 0.869 | 0.860 | 0.877 | 0.910 | 0.856 | 0.964 | 0.966 | 0.930 | 1 |
| OAS1 SIGLEC1 ISG15 HERC6 SLC1A2 RETN | 0.869 | 0.859 | 0.879 | 0.905 | 0.844 | 0.965 | 0.952 | 0.902 | 1 |
| IFI27 ISG15 SLC1A2 FAM20A RETN MMP8 | 0.869 | 0.858 | 0.879 | 0.905 | 0.842 | 0.968 | 0.928 | 0.842 | 1 |
| OAS1 IFIT1 SIGLEC1 ISG15 SLC1A2 IL1R2 | 0.869 | 0.860 | 0.878 | 0.908 | 0.853 | 0.964 | 0.964 | 0.927 | 1 |
| IFI27 ISG15 HERC6 SLC1A2 FAM20A MMP8 | 0.869 | 0.858 | 0.879 | 0.907 | 0.848 | 0.966 | 0.929 | 0.846 | 1 |
| IFI44L SIGLEC1 ISG15 IL1R2 RETN MMP8 | 0.869 | 0.859 | 0.878 | 0.902 | 0.840 | 0.965 | 0.963 | 0.925 | 1 |
| RSAD2 SLC1A2 IL1R2 OLAH FAM20A RETN | 0.869 | 0.859 | 0.878 | 0.910 | 0.854 | 0.967 | 0.930 | 0.849 | 1 |
| RSAD2 HERC6 SLC1A2 OLAH FAM20A RETN | 0.869 | 0.859 | 0.879 | 0.910 | 0.853 | 0.967 | 0.933 | 0.852 | 1 |
| RSAD2 OAS1 IFI44L SLC1A2 OLAH FAM20A | 0.869 | 0.859 | 0.879 | 0.910 | 0.852 | 0.969 | 0.924 | 0.848 | 1.000 |
| IFI27 IFIT1 SIGLEC1 ISG15 SLC1A2 MMP8 | 0.869 | 0.860 | 0.878 | 0.904 | 0.845 | 0.962 | 0.924 | 0.854 | 0.994 |
| RSAD2 IFI27 OAS1 IFIT1 IFI44L FAM20A | 0.869 | 0.858 | 0.879 | 0.901 | 0.835 | 0.968 | 0.938 | 0.862 | 1 |
| RSAD2 SIGLEC1 HERC6 IL1R2 OLAH RETN | 0.869 | 0.859 | 0.878 | 0.902 | 0.838 | 0.966 | 0.953 | 0.909 | 0.998 |
| RSAD2 OAS1 IFIT1 IFI44L IL1R2 FAM20A | 0.869 | 0.859 | 0.878 | 0.901 | 0.836 | 0.966 | 0.940 | 0.866 | 1 |
| OAS1 SLC1A2 IL1R2 OLAH FAM20A RETN | 0.869 | 0.859 | 0.878 | 0.912 | 0.856 | 0.969 | 0.926 | 0.842 | 1 |
| OAS1 IFIT1 IFI44L IL1R2 OLAH FAM20A | 0.869 | 0.859 | 0.878 | 0.900 | 0.834 | 0.967 | 0.947 | 0.879 | 1 |
| RSAD2 IFI27 OAS1 IFI44L ISG15 RETN | 0.869 | 0.859 | 0.878 | 0.906 | 0.843 | 0.969 | 0.952 | 0.890 | 1 |
| IFIT1 HERC6 SLC1A2 IL1R2 FAM20A RETN | 0.869 | 0.859 | 0.879 | 0.911 | 0.857 | 0.965 | 0.930 | 0.846 | 1 |
| OAS1 IFIT1 IFI44L SIGLEC1 HERC6 OLAH | 0.868 | 0.859 | 0.878 | 0.899 | 0.838 | 0.961 | 0.936 | 0.881 | 0.991 |
| OAS1 HERC6 SLC1A2 IL1R2 FAM20A RETN | 0.868 | 0.859 | 0.878 | 0.912 | 0.858 | 0.966 | 0.925 | 0.836 | 1 |
| OAS1 IFIT1 SIGLEC1 ISG15 IL1R2 OLAH | 0.868 | 0.860 | 0.877 | 0.901 | 0.839 | 0.964 | 0.957 | 0.914 | 0.999 |
| OAS1 ISG15 HERC6 OLAH FAM20A RETN | 0.868 | 0.858 | 0.878 | 0.904 | 0.842 | 0.967 | 0.947 | 0.882 | 1 |
| RSAD2 HERC6 SLC1A2 IL1R2 FAM20A RETN | 0.868 | 0.859 | 0.878 | 0.911 | 0.856 | 0.965 | 0.929 | 0.840 | 1 |
| OAS1 IFIT1 IFI44L SIGLEC1 ISG15 IL1R2 | 0.868 | 0.860 | 0.877 | 0.899 | 0.836 | 0.963 | 0.957 | 0.915 | 0.998 |
| RSAD2 IFIT1 SIGLEC1 SLC1A2 IL1R2 MMP8 | 0.868 | 0.860 | 0.877 | 0.909 | 0.854 | 0.963 | 0.965 | 0.929 | 1 |
| RSAD2 OAS1 ISG15 IL1R2 FAM20A MMP8 | 0.868 | 0.859 | 0.878 | 0.903 | 0.841 | 0.966 | 0.934 | 0.853 | 1 |
| RSAD2 OAS1 IFIT1 ISG15 IL1R2 FAM20A | 0.868 | 0.859 | 0.878 | 0.901 | 0.836 | 0.966 | 0.942 | 0.868 | 1 |
| SIGLEC1 ISG15 SLC1A2 IL1R2 RETN MMP8 | 0.868 | 0.860 | 0.877 | 0.912 | 0.858 | 0.966 | 0.970 | 0.937 | 1 |
| IFI44L SIGLEC1 ISG15 HERC6 SLC1A2 IL1R2 | 0.868 | 0.860 | 0.877 | 0.909 | 0.854 | 0.964 | 0.961 | 0.921 | 1 |
| HERC6 SLC1A2 IL1R2 FAM20A RETN MMP8 | 0.868 | 0.859 | 0.878 | 0.913 | 0.860 | 0.966 | 0.924 | 0.831 | 1 |
| OAS1 IFIT1 SLC1A2 OLAH FAM20A MMP8 | 0.868 | 0.858 | 0.878 | 0.909 | 0.850 | 0.967 | 0.940 | 0.870 | 1 |
| RSAD2 OAS1 SIGLEC1 ISG15 FAM20A MMP8 | 0.868 | 0.858 | 0.879 | 0.895 | 0.824 | 0.966 | 0.950 | 0.885 | 1 |
| IFIT1 IFI44L SIGLEC1 ISG15 HERC6 OLAH | 0.868 | 0.859 | 0.878 | 0.900 | 0.839 | 0.962 | 0.942 | 0.892 | 0.993 |
| IFIT1 IFI44L SIGLEC1 ISG15 SLC1A2 IL1R2 | 0.868 | 0.860 | 0.877 | 0.907 | 0.852 | 0.963 | 0.965 | 0.929 | 1 |
| RSAD2 IFI44L SIGLEC1 HERC6 FAM20A MMP8 | 0.868 | 0.858 | 0.878 | 0.899 | 0.833 | 0.964 | 0.942 | 0.873 | 1 |
| OAS1 IFI44L SLC1A2 IL1R2 FAM20A MMP8 | 0.868 | 0.859 | 0.878 | 0.913 | 0.862 | 0.965 | 0.927 | 0.834 | 1 |
| IFI44L SIGLEC1 ISG15 HERC6 FAM20A MMP8 | 0.868 | 0.858 | 0.878 | 0.898 | 0.831 | 0.966 | 0.953 | 0.882 | 1 |
| OAS1 IFI44L SIGLEC1 ISG15 HERC6 FAM20A | 0.868 | 0.858 | 0.878 | 0.899 | 0.834 | 0.965 | 0.953 | 0.891 | 1 |
| IFIT1 HERC6 SLC1A2 OLAH FAM20A RETN | 0.868 | 0.858 | 0.878 | 0.911 | 0.854 | 0.967 | 0.930 | 0.850 | 1 |
| IFIT1 IFI44L ISG15 IL1R2 FAM20A MMP8 | 0.868 | 0.859 | 0.878 | 0.904 | 0.841 | 0.966 | 0.942 | 0.867 | 1 |
| RSAD2 IFI27 OAS1 IFIT1 IFI44L RETN | 0.868 | 0.858 | 0.878 | 0.906 | 0.842 | 0.969 | 0.949 | 0.884 | 1 |
| RSAD2 OAS1 IFIT1 SIGLEC1 IL1R2 OLAH | 0.868 | 0.859 | 0.877 | 0.899 | 0.836 | 0.963 | 0.959 | 0.917 | 1 |
| RSAD2 OAS1 IFIT1 SIGLEC1 SLC1A2 IL1R2 | 0.868 | 0.859 | 0.877 | 0.907 | 0.852 | 0.963 | 0.968 | 0.932 | 1 |
| RSAD2 OAS1 IFI44L ISG15 IL1R2 FAM20A | 0.868 | 0.858 | 0.878 | 0.903 | 0.840 | 0.966 | 0.939 | 0.864 | 1 |
| RSAD2 OAS1 IFI44L SIGLEC1 SLC1A2 FAM20A | 0.868 | 0.858 | 0.878 | 0.904 | 0.842 | 0.966 | 0.945 | 0.873 | 1 |
| IFIT1 IFI44L SIGLEC1 IL1R2 RETN MMP8 | 0.868 | 0.858 | 0.878 | 0.900 | 0.837 | 0.964 | 0.962 | 0.923 | 1 |
| RSAD2 SIGLEC1 ISG15 SLC1A2 FAM20A RETN | 0.868 | 0.858 | 0.878 | 0.903 | 0.841 | 0.966 | 0.947 | 0.874 | 1 |
| RSAD2 IFIT1 SLC1A2 IL1R2 OLAH FAM20A | 0.868 | 0.859 | 0.877 | 0.908 | 0.851 | 0.966 | 0.933 | 0.855 | 1 |
| RSAD2 IFI27 IFIT1 IFI44L FAM20A MMP8 | 0.868 | 0.858 | 0.879 | 0.899 | 0.833 | 0.966 | 0.934 | 0.856 | 1 |
| RSAD2 OAS1 SIGLEC1 SLC1A2 IL1R2 RETN | 0.868 | 0.859 | 0.877 | 0.909 | 0.854 | 0.965 | 0.968 | 0.931 | 1 |
| OAS1 IFIT1 HERC6 OLAH FAM20A RETN | 0.868 | 0.858 | 0.878 | 0.901 | 0.836 | 0.966 | 0.945 | 0.878 | 1 |
| OAS1 SIGLEC1 ISG15 SLC1A2 FAM20A RETN | 0.868 | 0.858 | 0.878 | 0.901 | 0.838 | 0.965 | 0.947 | 0.874 | 1 |
| RSAD2 OAS1 SIGLEC1 ISG15 HERC6 RETN | 0.868 | 0.858 | 0.878 | 0.897 | 0.831 | 0.964 | 0.949 | 0.901 | 0.997 |
| RSAD2 IFI27 IFIT1 IFI44L ISG15 FAM20A | 0.868 | 0.858 | 0.878 | 0.903 | 0.835 | 0.970 | 0.943 | 0.870 | 1 |
| IFIT1 IFI44L SIGLEC1 ISG15 IL1R2 MMP8 | 0.868 | 0.859 | 0.877 | 0.901 | 0.842 | 0.961 | 0.960 | 0.919 | 1 |
| SIGLEC1 ISG15 HERC6 SLC1A2 IL1R2 RETN | 0.868 | 0.859 | 0.877 | 0.909 | 0.853 | 0.965 | 0.960 | 0.919 | 1 |
| RSAD2 IFI44L SIGLEC1 SLC1A2 OLAH RETN | 0.868 | 0.859 | 0.878 | 0.913 | 0.857 | 0.969 | 0.950 | 0.901 | 0.999 |
| RSAD2 OAS1 SIGLEC1 SLC1A2 FAM20A RETN | 0.868 | 0.858 | 0.878 | 0.903 | 0.840 | 0.966 | 0.947 | 0.874 | 1 |
| OAS1 IFIT1 IL1R2 OLAH FAM20A RETN | 0.868 | 0.858 | 0.878 | 0.900 | 0.834 | 0.966 | 0.941 | 0.868 | 1 |
| RSAD2 IFIT1 SIGLEC1 ISG15 HERC6 RETN | 0.868 | 0.859 | 0.877 | 0.898 | 0.833 | 0.963 | 0.929 | 0.872 | 0.987 |
| RSAD2 IFIT1 IFI44L SIGLEC1 IL1R2 MMP8 | 0.868 | 0.859 | 0.877 | 0.900 | 0.839 | 0.962 | 0.959 | 0.917 | 1 |
| RSAD2 IFI44L SIGLEC1 ISG15 SLC1A2 OLAH | 0.868 | 0.859 | 0.877 | 0.911 | 0.855 | 0.967 | 0.949 | 0.901 | 0.997 |
| RSAD2 SIGLEC1 SLC1A2 IL1R2 RETN MMP8 | 0.868 | 0.859 | 0.877 | 0.913 | 0.859 | 0.966 | 0.973 | 0.943 | 1 |
| IFI27 OAS1 SIGLEC1 ISG15 SLC1A2 MMP8 | 0.868 | 0.859 | 0.877 | 0.905 | 0.847 | 0.963 | 0.932 | 0.864 | 0.999 |
| RSAD2 IFI44L SIGLEC1 ISG15 SLC1A2 FAM20A | 0.868 | 0.858 | 0.878 | 0.903 | 0.840 | 0.965 | 0.942 | 0.870 | 1 |
| OAS1 HERC6 SLC1A2 OLAH FAM20A RETN | 0.868 | 0.858 | 0.878 | 0.911 | 0.854 | 0.967 | 0.928 | 0.847 | 1 |
| IFIT1 SLC1A2 IL1R2 FAM20A RETN MMP8 | 0.868 | 0.859 | 0.877 | 0.911 | 0.855 | 0.966 | 0.938 | 0.861 | 1 |
| RSAD2 IFI44L SIGLEC1 HERC6 SLC1A2 IL1R2 | 0.868 | 0.859 | 0.877 | 0.908 | 0.853 | 0.964 | 0.960 | 0.919 | 1 |
| RSAD2 IFI27 OAS1 SIGLEC1 SLC1A2 MMP8 | 0.868 | 0.859 | 0.877 | 0.903 | 0.844 | 0.963 | 0.939 | 0.875 | 1 |
| RSAD2 OAS1 IFI44L SIGLEC1 IL1R2 OLAH | 0.868 | 0.859 | 0.877 | 0.898 | 0.833 | 0.963 | 0.961 | 0.921 | 1 |
| IFIT1 ISG15 HERC6 OLAH FAM20A MMP8 | 0.868 | 0.858 | 0.878 | 0.900 | 0.837 | 0.963 | 0.942 | 0.873 | 1 |
| OAS1 IFIT1 ISG15 SLC1A2 IL1R2 FAM20A | 0.868 | 0.859 | 0.877 | 0.908 | 0.851 | 0.965 | 0.936 | 0.855 | 1 |
| SIGLEC1 ISG15 IL1R2 OLAH RETN MMP8 | 0.868 | 0.858 | 0.877 | 0.902 | 0.839 | 0.965 | 0.970 | 0.937 | 1 |
| RSAD2 IFI44L SIGLEC1 IL1R2 OLAH MMP8 | 0.868 | 0.859 | 0.877 | 0.901 | 0.841 | 0.962 | 0.960 | 0.919 | 1 |
| OAS1 IFI44L SIGLEC1 IL1R2 RETN MMP8 | 0.868 | 0.858 | 0.877 | 0.902 | 0.839 | 0.965 | 0.960 | 0.920 | 1.000 |
| OAS1 SIGLEC1 SLC1A2 IL1R2 RETN MMP8 | 0.868 | 0.859 | 0.877 | 0.912 | 0.859 | 0.966 | 0.971 | 0.939 | 1 |
| RSAD2 OAS1 IFI44L HERC6 OLAH FAM20A | 0.868 | 0.858 | 0.878 | 0.901 | 0.838 | 0.965 | 0.938 | 0.871 | 1 |
| IFIT1 IFI44L SIGLEC1 SLC1A2 IL1R2 MMP8 | 0.868 | 0.859 | 0.876 | 0.908 | 0.854 | 0.962 | 0.964 | 0.926 | 1 |
| RSAD2 IFIT1 HERC6 SLC1A2 IL1R2 FAM20A | 0.868 | 0.858 | 0.877 | 0.913 | 0.859 | 0.966 | 0.930 | 0.845 | 1 |
| OAS1 SIGLEC1 ISG15 SLC1A2 IL1R2 RETN | 0.868 | 0.859 | 0.877 | 0.910 | 0.854 | 0.965 | 0.965 | 0.927 | 1 |
| IFI27 IFIT1 IFI44L SLC1A2 FAM20A MMP8 | 0.868 | 0.858 | 0.878 | 0.901 | 0.836 | 0.965 | 0.932 | 0.850 | 1 |
| IFI27 IFI44L ISG15 SLC1A2 FAM20A MMP8 | 0.868 | 0.857 | 0.878 | 0.900 | 0.836 | 0.965 | 0.937 | 0.857 | 1 |
| IFIT1 IFI44L SIGLEC1 SLC1A2 IL1R2 RETN | 0.868 | 0.859 | 0.877 | 0.910 | 0.855 | 0.965 | 0.962 | 0.922 | 1 |
| RSAD2 IFI44L SIGLEC1 FAM20A RETN MMP8 | 0.868 | 0.858 | 0.878 | 0.901 | 0.833 | 0.969 | 0.954 | 0.895 | 1 |
| RSAD2 OAS1 IFI44L SIGLEC1 ISG15 FAM20A | 0.868 | 0.858 | 0.878 | 0.898 | 0.827 | 0.968 | 0.952 | 0.891 | 1 |
| RSAD2 OAS1 SIGLEC1 IL1R2 RETN MMP8 | 0.868 | 0.858 | 0.878 | 0.899 | 0.835 | 0.964 | 0.971 | 0.938 | 1 |
| IFIT1 SIGLEC1 IL1R2 OLAH RETN MMP8 | 0.868 | 0.858 | 0.877 | 0.903 | 0.841 | 0.964 | 0.965 | 0.929 | 1 |
| RSAD2 OAS1 IFIT1 IFI44L SIGLEC1 IL1R2 | 0.868 | 0.858 | 0.877 | 0.897 | 0.831 | 0.962 | 0.957 | 0.915 | 0.998 |
| RSAD2 IFI27 OAS1 ISG15 HERC6 MMP8 | 0.868 | 0.859 | 0.877 | 0.901 | 0.843 | 0.958 | 0.903 | 0.827 | 0.980 |
| OAS1 SIGLEC1 HERC6 OLAH RETN MMP8 | 0.868 | 0.857 | 0.878 | 0.896 | 0.828 | 0.963 | 0.964 | 0.926 | 1 |
| RSAD2 IFIT1 IFI44L SIGLEC1 IL1R2 OLAH | 0.868 | 0.859 | 0.877 | 0.899 | 0.834 | 0.964 | 0.958 | 0.916 | 0.999 |
| RSAD2 SIGLEC1 ISG15 SLC1A2 IL1R2 MMP8 | 0.868 | 0.859 | 0.876 | 0.909 | 0.854 | 0.964 | 0.966 | 0.931 | 1 |
| RSAD2 ISG15 IL1R2 OLAH FAM20A MMP8 | 0.868 | 0.858 | 0.877 | 0.902 | 0.839 | 0.964 | 0.941 | 0.868 | 1 |
| RSAD2 ISG15 HERC6 OLAH FAM20A RETN | 0.868 | 0.858 | 0.878 | 0.905 | 0.844 | 0.967 | 0.942 | 0.875 | 1 |
| OAS1 IFIT1 HERC6 OLAH FAM20A MMP8 | 0.868 | 0.858 | 0.878 | 0.899 | 0.834 | 0.963 | 0.941 | 0.873 | 1 |
| IFI27 OAS1 IFI44L HERC6 SLC1A2 FAM20A | 0.868 | 0.857 | 0.878 | 0.910 | 0.852 | 0.967 | 0.933 | 0.851 | 1 |
| RSAD2 OAS1 HERC6 OLAH FAM20A MMP8 | 0.868 | 0.858 | 0.877 | 0.899 | 0.835 | 0.963 | 0.941 | 0.874 | 1 |
| OAS1 ISG15 IL1R2 OLAH FAM20A RETN | 0.868 | 0.858 | 0.877 | 0.900 | 0.834 | 0.965 | 0.930 | 0.852 | 1 |
| IFI44L ISG15 HERC6 OLAH FAM20A MMP8 | 0.868 | 0.858 | 0.878 | 0.899 | 0.833 | 0.964 | 0.948 | 0.882 | 1 |
| RSAD2 IFIT1 HERC6 SLC1A2 OLAH FAM20A | 0.868 | 0.857 | 0.878 | 0.910 | 0.854 | 0.966 | 0.930 | 0.849 | 1 |
| OAS1 SIGLEC1 ISG15 SLC1A2 FAM20A MMP8 | 0.868 | 0.857 | 0.878 | 0.902 | 0.839 | 0.965 | 0.949 | 0.878 | 1 |
| OAS1 IFIT1 SIGLEC1 IL1R2 OLAH RETN | 0.868 | 0.858 | 0.877 | 0.900 | 0.834 | 0.965 | 0.955 | 0.912 | 0.999 |
| IFI27 OAS1 ISG15 HERC6 SLC1A2 FAM20A | 0.868 | 0.857 | 0.878 | 0.905 | 0.846 | 0.964 | 0.929 | 0.845 | 1 |
| RSAD2 IFIT1 SIGLEC1 SLC1A2 IL1R2 RETN | 0.868 | 0.858 | 0.877 | 0.908 | 0.852 | 0.964 | 0.965 | 0.928 | 1 |
| IFIT1 SIGLEC1 ISG15 IL1R2 OLAH MMP8 | 0.868 | 0.859 | 0.876 | 0.905 | 0.848 | 0.962 | 0.963 | 0.925 | 1 |
| RSAD2 OAS1 SIGLEC1 SLC1A2 IL1R2 MMP8 | 0.867 | 0.858 | 0.877 | 0.910 | 0.856 | 0.963 | 0.968 | 0.934 | 1 |
| OAS1 IFI44L SIGLEC1 SLC1A2 IL1R2 MMP8 | 0.867 | 0.859 | 0.876 | 0.908 | 0.854 | 0.963 | 0.965 | 0.928 | 1 |
| IFI27 OAS1 IFI44L ISG15 HERC6 MMP8 | 0.867 | 0.859 | 0.876 | 0.901 | 0.845 | 0.958 | 0.900 | 0.822 | 0.978 |
| IFI27 OAS1 IFIT1 ISG15 SLC1A2 RETN | 0.867 | 0.858 | 0.877 | 0.899 | 0.834 | 0.964 | 0.941 | 0.869 | 1 |
| RSAD2 IFIT1 SIGLEC1 IL1R2 OLAH MMP8 | 0.867 | 0.858 | 0.877 | 0.905 | 0.846 | 0.963 | 0.964 | 0.927 | 1 |
| IFI27 OAS1 IFIT1 IFI44L FAM20A MMP8 | 0.867 | 0.857 | 0.878 | 0.897 | 0.830 | 0.965 | 0.934 | 0.854 | 1 |
| RSAD2 OAS1 IFI44L SIGLEC1 FAM20A MMP8 | 0.867 | 0.857 | 0.878 | 0.896 | 0.826 | 0.967 | 0.951 | 0.888 | 1 |
| RSAD2 IFI44L ISG15 SLC1A2 IL1R2 FAM20A | 0.867 | 0.858 | 0.877 | 0.907 | 0.851 | 0.963 | 0.930 | 0.844 | 1 |
| IFIT1 IFI44L ISG15 IL1R2 OLAH FAM20A | 0.867 | 0.858 | 0.877 | 0.900 | 0.835 | 0.965 | 0.952 | 0.895 | 1 |
| RSAD2 OAS1 HERC6 OLAH FAM20A RETN | 0.867 | 0.858 | 0.877 | 0.902 | 0.837 | 0.966 | 0.939 | 0.871 | 1 |
| RSAD2 OAS1 IFI44L SIGLEC1 IL1R2 MMP8 | 0.867 | 0.858 | 0.876 | 0.901 | 0.840 | 0.962 | 0.961 | 0.921 | 1 |
| IFI44L SIGLEC1 HERC6 OLAH RETN MMP8 | 0.867 | 0.857 | 0.878 | 0.896 | 0.827 | 0.965 | 0.962 | 0.924 | 1 |
| IFIT1 ISG15 SLC1A2 IL1R2 FAM20A RETN | 0.867 | 0.858 | 0.877 | 0.905 | 0.848 | 0.962 | 0.932 | 0.851 | 1 |
| RSAD2 OAS1 SIGLEC1 ISG15 SLC1A2 IL1R2 | 0.867 | 0.858 | 0.877 | 0.908 | 0.852 | 0.963 | 0.966 | 0.928 | 1 |
| OAS1 SIGLEC1 IL1R2 OLAH RETN MMP8 | 0.867 | 0.857 | 0.877 | 0.900 | 0.836 | 0.964 | 0.970 | 0.936 | 1 |
| OAS1 HERC6 OLAH FAM20A RETN MMP8 | 0.867 | 0.857 | 0.877 | 0.900 | 0.835 | 0.965 | 0.950 | 0.886 | 1 |
| RSAD2 SIGLEC1 ISG15 SLC1A2 IL1R2 RETN | 0.867 | 0.858 | 0.876 | 0.910 | 0.855 | 0.965 | 0.964 | 0.926 | 1 |
| OAS1 SIGLEC1 ISG15 SLC1A2 IL1R2 MMP8 | 0.867 | 0.859 | 0.876 | 0.910 | 0.856 | 0.964 | 0.966 | 0.930 | 1 |
| OAS1 IFIT1 ISG15 HERC6 OLAH FAM20A | 0.867 | 0.857 | 0.877 | 0.900 | 0.837 | 0.963 | 0.941 | 0.872 | 1 |
| RSAD2 IFI44L SLC1A2 OLAH FAM20A RETN | 0.867 | 0.857 | 0.877 | 0.909 | 0.850 | 0.968 | 0.926 | 0.852 | 1 |
| RSAD2 OAS1 IFI44L SIGLEC1 FAM20A RETN | 0.867 | 0.857 | 0.877 | 0.896 | 0.825 | 0.966 | 0.953 | 0.894 | 1 |
| OAS1 IFIT1 ISG15 HERC6 FAM20A RETN | 0.867 | 0.857 | 0.877 | 0.898 | 0.834 | 0.962 | 0.937 | 0.860 | 1 |
| OAS1 IFIT1 IFI44L SLC1A2 IL1R2 FAM20A | 0.867 | 0.858 | 0.876 | 0.906 | 0.849 | 0.963 | 0.925 | 0.837 | 1 |
| OAS1 IFIT1 ISG15 IL1R2 FAM20A RETN | 0.867 | 0.857 | 0.877 | 0.900 | 0.835 | 0.965 | 0.945 | 0.871 | 1 |
| OAS1 IFIT1 IFI44L SIGLEC1 IL1R2 RETN | 0.867 | 0.858 | 0.877 | 0.898 | 0.832 | 0.964 | 0.957 | 0.915 | 0.998 |
| RSAD2 OAS1 ISG15 HERC6 OLAH FAM20A | 0.867 | 0.857 | 0.877 | 0.900 | 0.837 | 0.963 | 0.938 | 0.868 | 1 |
| RSAD2 IFIT1 SIGLEC1 ISG15 SLC1A2 IL1R2 | 0.867 | 0.859 | 0.876 | 0.905 | 0.850 | 0.961 | 0.964 | 0.927 | 1 |
| OAS1 IFIT1 IFI44L ISG15 IL1R2 FAM20A | 0.867 | 0.858 | 0.877 | 0.900 | 0.835 | 0.965 | 0.945 | 0.871 | 1 |
| IFI27 OAS1 IFIT1 ISG15 RETN MMP8 | 0.867 | 0.857 | 0.877 | 0.903 | 0.839 | 0.967 | 0.950 | 0.887 | 1 |
| RSAD2 IFI44L SIGLEC1 OLAH FAM20A RETN | 0.867 | 0.857 | 0.877 | 0.898 | 0.827 | 0.969 | 0.961 | 0.917 | 1 |
| RSAD2 IFIT1 SLC1A2 OLAH FAM20A MMP8 | 0.867 | 0.857 | 0.877 | 0.905 | 0.846 | 0.965 | 0.939 | 0.869 | 1 |
| OAS1 IFIT1 SIGLEC1 ISG15 HERC6 OLAH | 0.867 | 0.858 | 0.877 | 0.900 | 0.839 | 0.962 | 0.941 | 0.890 | 0.993 |
| RSAD2 IFI27 ISG15 HERC6 SLC1A2 FAM20A | 0.867 | 0.857 | 0.877 | 0.908 | 0.850 | 0.965 | 0.927 | 0.843 | 1 |
| RSAD2 IFIT1 IFI44L SIGLEC1 HERC6 RETN | 0.867 | 0.858 | 0.876 | 0.895 | 0.826 | 0.964 | 0.922 | 0.860 | 0.983 |
| RSAD2 IFI44L ISG15 HERC6 OLAH FAM20A | 0.867 | 0.857 | 0.877 | 0.903 | 0.841 | 0.965 | 0.941 | 0.876 | 1 |
| OAS1 IFI44L SIGLEC1 ISG15 FAM20A RETN | 0.867 | 0.857 | 0.877 | 0.895 | 0.823 | 0.966 | 0.955 | 0.898 | 1 |
| IFI27 IFI44L SIGLEC1 ISG15 SLC1A2 MMP8 | 0.867 | 0.858 | 0.876 | 0.901 | 0.841 | 0.960 | 0.925 | 0.853 | 0.997 |
| IFIT1 SIGLEC1 ISG15 IL1R2 RETN MMP8 | 0.867 | 0.857 | 0.877 | 0.902 | 0.839 | 0.964 | 0.968 | 0.935 | 1 |
| OAS1 IFIT1 IFI44L SIGLEC1 SLC1A2 IL1R2 | 0.867 | 0.858 | 0.876 | 0.907 | 0.851 | 0.963 | 0.963 | 0.925 | 1 |
| RSAD2 OAS1 IL1R2 FAM20A RETN MMP8 | 0.867 | 0.857 | 0.877 | 0.904 | 0.841 | 0.967 | 0.938 | 0.859 | 1 |
| RSAD2 IFI44L ISG15 HERC6 FAM20A RETN | 0.867 | 0.857 | 0.877 | 0.905 | 0.844 | 0.966 | 0.935 | 0.859 | 1 |
| RSAD2 IFI44L SIGLEC1 SLC1A2 IL1R2 MMP8 | 0.867 | 0.858 | 0.876 | 0.907 | 0.852 | 0.962 | 0.968 | 0.934 | 1 |
| IFIT1 SLC1A2 IL1R2 OLAH FAM20A RETN | 0.867 | 0.857 | 0.877 | 0.907 | 0.850 | 0.964 | 0.929 | 0.852 | 1 |
| RSAD2 OAS1 IFIT1 HERC6 OLAH FAM20A | 0.867 | 0.857 | 0.877 | 0.901 | 0.837 | 0.965 | 0.936 | 0.865 | 1 |
| OAS1 IFI44L SLC1A2 OLAH FAM20A RETN | 0.867 | 0.857 | 0.877 | 0.908 | 0.849 | 0.968 | 0.927 | 0.852 | 1 |
| RSAD2 IFIT1 IFI44L SIGLEC1 ISG15 IL1R2 | 0.867 | 0.858 | 0.876 | 0.896 | 0.831 | 0.961 | 0.955 | 0.913 | 0.998 |
| RSAD2 IFI44L SIGLEC1 SLC1A2 IL1R2 RETN | 0.867 | 0.858 | 0.876 | 0.907 | 0.850 | 0.963 | 0.970 | 0.936 | 1 |
| RSAD2 IFI44L SIGLEC1 ISG15 IL1R2 MMP8 | 0.867 | 0.858 | 0.876 | 0.900 | 0.838 | 0.962 | 0.960 | 0.919 | 1 |
| ISG15 SLC1A2 IL1R2 OLAH FAM20A MMP8 | 0.867 | 0.858 | 0.876 | 0.910 | 0.856 | 0.964 | 0.923 | 0.840 | 1 |
| RSAD2 IFI44L IL1R2 OLAH FAM20A MMP8 | 0.867 | 0.857 | 0.876 | 0.904 | 0.842 | 0.965 | 0.948 | 0.884 | 1 |
| IFIT1 IFI44L SLC1A2 IL1R2 FAM20A RETN | 0.867 | 0.858 | 0.876 | 0.909 | 0.854 | 0.963 | 0.925 | 0.838 | 1 |
| IFI27 IFIT1 IFI44L ISG15 RETN MMP8 | 0.867 | 0.856 | 0.877 | 0.903 | 0.839 | 0.967 | 0.953 | 0.897 | 1 |
| RSAD2 OAS1 IFI44L SLC1A2 IL1R2 FAM20A | 0.867 | 0.857 | 0.876 | 0.907 | 0.851 | 0.964 | 0.925 | 0.837 | 1 |
| OAS1 IFI44L SIGLEC1 ISG15 IL1R2 OLAH | 0.867 | 0.858 | 0.876 | 0.901 | 0.837 | 0.965 | 0.951 | 0.905 | 0.997 |
| OAS1 IFI44L ISG15 SLC1A2 IL1R2 FAM20A | 0.867 | 0.857 | 0.876 | 0.907 | 0.851 | 0.964 | 0.925 | 0.837 | 1 |
| RSAD2 OAS1 IFIT1 SIGLEC1 HERC6 OLAH | 0.867 | 0.857 | 0.876 | 0.901 | 0.840 | 0.962 | 0.937 | 0.883 | 0.991 |
| IFIT1 IFI44L SIGLEC1 HERC6 RETN MMP8 | 0.867 | 0.857 | 0.876 | 0.894 | 0.824 | 0.964 | 0.925 | 0.865 | 0.985 |
| OAS1 SIGLEC1 ISG15 HERC6 FAM20A MMP8 | 0.867 | 0.857 | 0.877 | 0.898 | 0.832 | 0.964 | 0.949 | 0.885 | 1 |
| IFI27 OAS1 IFI44L ISG15 RETN MMP8 | 0.867 | 0.857 | 0.877 | 0.905 | 0.842 | 0.968 | 0.953 | 0.895 | 1 |
| IFI44L SIGLEC1 ISG15 IL1R2 OLAH RETN | 0.867 | 0.858 | 0.876 | 0.900 | 0.837 | 0.964 | 0.960 | 0.917 | 1 |
| OAS1 IFI44L SIGLEC1 ISG15 IL1R2 MMP8 | 0.867 | 0.858 | 0.875 | 0.901 | 0.841 | 0.961 | 0.955 | 0.912 | 0.999 |
| OAS1 IFI44L SIGLEC1 SLC1A2 IL1R2 RETN | 0.867 | 0.857 | 0.876 | 0.910 | 0.855 | 0.965 | 0.962 | 0.922 | 1 |
| RSAD2 OAS1 IFIT1 SIGLEC1 IL1R2 MMP8 | 0.867 | 0.857 | 0.876 | 0.901 | 0.842 | 0.961 | 0.966 | 0.931 | 1 |
| IFI44L ISG15 IL1R2 OLAH FAM20A RETN | 0.867 | 0.857 | 0.876 | 0.901 | 0.838 | 0.965 | 0.943 | 0.879 | 1 |
| RSAD2 IFIT1 ISG15 IL1R2 FAM20A MMP8 | 0.867 | 0.857 | 0.876 | 0.905 | 0.843 | 0.967 | 0.943 | 0.870 | 1 |
| RSAD2 IFIT1 HERC6 OLAH FAM20A RETN | 0.867 | 0.857 | 0.877 | 0.902 | 0.839 | 0.966 | 0.943 | 0.877 | 1 |
| RSAD2 HERC6 OLAH FAM20A RETN MMP8 | 0.867 | 0.857 | 0.877 | 0.900 | 0.836 | 0.965 | 0.947 | 0.881 | 1 |
| RSAD2 OAS1 SIGLEC1 ISG15 IL1R2 OLAH | 0.867 | 0.857 | 0.876 | 0.899 | 0.836 | 0.962 | 0.959 | 0.917 | 1 |
| OAS1 IFI44L ISG15 IL1R2 FAM20A MMP8 | 0.867 | 0.857 | 0.876 | 0.904 | 0.842 | 0.966 | 0.938 | 0.858 | 1 |
| RSAD2 IFIT1 ISG15 SLC1A2 IL1R2 FAM20A | 0.867 | 0.858 | 0.875 | 0.904 | 0.845 | 0.963 | 0.934 | 0.852 | 1 |
| IFIT1 IFI44L SIGLEC1 ISG15 IL1R2 OLAH | 0.867 | 0.858 | 0.876 | 0.902 | 0.841 | 0.963 | 0.958 | 0.915 | 1 |
| RSAD2 OAS1 ISG15 HERC6 FAM20A RETN | 0.867 | 0.857 | 0.876 | 0.901 | 0.838 | 0.964 | 0.938 | 0.862 | 1 |
| OAS1 IFI44L SIGLEC1 SLC1A2 FAM20A MMP8 | 0.867 | 0.857 | 0.876 | 0.903 | 0.841 | 0.966 | 0.948 | 0.877 | 1 |
| OAS1 IFI44L SIGLEC1 ISG15 SLC1A2 IL1R2 | 0.867 | 0.858 | 0.876 | 0.907 | 0.852 | 0.963 | 0.964 | 0.927 | 1 |
| RSAD2 OAS1 IFI44L SIGLEC1 ISG15 IL1R2 | 0.867 | 0.857 | 0.876 | 0.895 | 0.829 | 0.961 | 0.960 | 0.920 | 1.000 |
| OAS1 IFI44L SIGLEC1 HERC6 FAM20A MMP8 | 0.867 | 0.857 | 0.876 | 0.899 | 0.833 | 0.965 | 0.953 | 0.891 | 1 |
| OAS1 SIGLEC1 ISG15 IL1R2 OLAH MMP8 | 0.867 | 0.857 | 0.876 | 0.904 | 0.845 | 0.963 | 0.963 | 0.926 | 1 |
| OAS1 IFIT1 SLC1A2 IL1R2 FAM20A RETN | 0.866 | 0.857 | 0.876 | 0.908 | 0.851 | 0.964 | 0.936 | 0.855 | 1 |
| RSAD2 IFI27 OAS1 IFI44L HERC6 MMP8 | 0.866 | 0.857 | 0.876 | 0.897 | 0.837 | 0.957 | 0.898 | 0.818 | 0.978 |
| RSAD2 IFI27 IFIT1 IFI44L ISG15 RETN | 0.866 | 0.857 | 0.876 | 0.903 | 0.839 | 0.967 | 0.953 | 0.895 | 1 |
| OAS1 ISG15 SLC1A2 OLAH FAM20A MMP8 | 0.866 | 0.857 | 0.876 | 0.910 | 0.853 | 0.967 | 0.936 | 0.861 | 1 |
| OAS1 IFIT1 SIGLEC1 ISG15 IL1R2 MMP8 | 0.866 | 0.858 | 0.875 | 0.901 | 0.842 | 0.961 | 0.963 | 0.925 | 1 |
| RSAD2 OAS1 IFIT1 SLC1A2 IL1R2 FAM20A | 0.866 | 0.858 | 0.875 | 0.906 | 0.848 | 0.964 | 0.936 | 0.855 | 1 |
| IFI27 OAS1 IFI44L ISG15 FAM20A MMP8 | 0.866 | 0.855 | 0.877 | 0.898 | 0.831 | 0.965 | 0.938 | 0.860 | 1 |
| RSAD2 IFIT1 IFI44L SIGLEC1 SLC1A2 IL1R2 | 0.866 | 0.858 | 0.875 | 0.907 | 0.850 | 0.963 | 0.965 | 0.929 | 1 |
| RSAD2 IFIT1 SIGLEC1 HERC6 OLAH MMP8 | 0.866 | 0.857 | 0.876 | 0.899 | 0.837 | 0.961 | 0.936 | 0.881 | 0.991 |
| OAS1 IFI44L HERC6 OLAH FAM20A RETN | 0.866 | 0.856 | 0.876 | 0.904 | 0.840 | 0.968 | 0.943 | 0.879 | 1 |
| RSAD2 IFI27 OAS1 HERC6 SLC1A2 MMP8 | 0.866 | 0.857 | 0.876 | 0.907 | 0.852 | 0.962 | 0.900 | 0.817 | 0.983 |
| IFI27 OAS1 IFIT1 IFI44L RETN MMP8 | 0.866 | 0.856 | 0.877 | 0.904 | 0.841 | 0.968 | 0.949 | 0.885 | 1 |
| RSAD2 SIGLEC1 ISG15 IL1R2 OLAH MMP8 | 0.866 | 0.857 | 0.876 | 0.903 | 0.844 | 0.962 | 0.966 | 0.931 | 1 |
| RSAD2 IFI44L SIGLEC1 ISG15 SLC1A2 IL1R2 | 0.866 | 0.858 | 0.875 | 0.906 | 0.849 | 0.963 | 0.967 | 0.933 | 1 |
| IFI27 IFI44L ISG15 HERC6 SLC1A2 FAM20A | 0.866 | 0.856 | 0.877 | 0.902 | 0.841 | 0.963 | 0.938 | 0.856 | 1 |
| RSAD2 IFI44L SIGLEC1 IL1R2 RETN MMP8 | 0.866 | 0.857 | 0.876 | 0.900 | 0.836 | 0.964 | 0.967 | 0.933 | 1 |
| RSAD2 OAS1 IFIT1 IL1R2 FAM20A RETN | 0.866 | 0.857 | 0.876 | 0.901 | 0.837 | 0.966 | 0.943 | 0.869 | 1 |
| RSAD2 OAS1 ISG15 SLC1A2 OLAH FAM20A | 0.866 | 0.856 | 0.876 | 0.910 | 0.852 | 0.968 | 0.935 | 0.862 | 1 |
| SIGLEC1 ISG15 HERC6 IL1R2 OLAH RETN | 0.866 | 0.856 | 0.876 | 0.900 | 0.835 | 0.966 | 0.959 | 0.918 | 1.000 |
| IFI27 OAS1 IFI44L SIGLEC1 SLC1A2 MMP8 | 0.866 | 0.857 | 0.875 | 0.903 | 0.845 | 0.962 | 0.932 | 0.864 | 0.999 |
| ISG15 SLC1A2 IL1R2 OLAH FAM20A RETN | 0.866 | 0.857 | 0.876 | 0.908 | 0.851 | 0.965 | 0.924 | 0.844 | 1 |
| RSAD2 SIGLEC1 IL1R2 OLAH RETN MMP8 | 0.866 | 0.856 | 0.876 | 0.900 | 0.837 | 0.964 | 0.970 | 0.937 | 1 |
| IFI44L IL1R2 OLAH FAM20A RETN MMP8 | 0.866 | 0.857 | 0.876 | 0.902 | 0.840 | 0.964 | 0.947 | 0.885 | 1 |
| IFIT1 SIGLEC1 ISG15 HERC6 RETN MMP8 | 0.866 | 0.856 | 0.876 | 0.896 | 0.829 | 0.963 | 0.934 | 0.878 | 0.989 |
| RSAD2 OAS1 IFIT1 SIGLEC1 IL1R2 RETN | 0.866 | 0.857 | 0.876 | 0.896 | 0.830 | 0.962 | 0.960 | 0.918 | 1 |
| OAS1 IFIT1 SIGLEC1 ISG15 IL1R2 RETN | 0.866 | 0.857 | 0.875 | 0.897 | 0.831 | 0.962 | 0.960 | 0.920 | 1.000 |
| RSAD2 IFIT1 SIGLEC1 IL1R2 RETN MMP8 | 0.866 | 0.857 | 0.876 | 0.902 | 0.839 | 0.965 | 0.968 | 0.935 | 1 |
| IFIT1 ISG15 SLC1A2 OLAH FAM20A MMP8 | 0.866 | 0.856 | 0.876 | 0.907 | 0.847 | 0.966 | 0.939 | 0.869 | 1 |
| IFIT1 IL1R2 OLAH FAM20A RETN MMP8 | 0.866 | 0.856 | 0.876 | 0.905 | 0.844 | 0.967 | 0.948 | 0.882 | 1 |
| RSAD2 IFI27 IFI44L SLC1A2 FAM20A MMP8 | 0.866 | 0.856 | 0.877 | 0.903 | 0.839 | 0.966 | 0.936 | 0.855 | 1 |
| RSAD2 OAS1 SLC1A2 IL1R2 FAM20A MMP8 | 0.866 | 0.857 | 0.875 | 0.910 | 0.856 | 0.963 | 0.927 | 0.835 | 1 |
| IFIT1 ISG15 IL1R2 FAM20A RETN MMP8 | 0.866 | 0.856 | 0.876 | 0.905 | 0.842 | 0.968 | 0.942 | 0.869 | 1 |
| IFIT1 IFI44L SIGLEC1 ISG15 IL1R2 RETN | 0.866 | 0.857 | 0.875 | 0.900 | 0.837 | 0.963 | 0.960 | 0.917 | 1 |
| RSAD2 SIGLEC1 HERC6 OLAH RETN MMP8 | 0.866 | 0.856 | 0.876 | 0.898 | 0.831 | 0.966 | 0.958 | 0.916 | 0.999 |
| RSAD2 IFI27 IFI44L ISG15 SLC1A2 FAM20A | 0.866 | 0.855 | 0.877 | 0.901 | 0.836 | 0.966 | 0.939 | 0.860 | 1 |
| RSAD2 OAS1 SIGLEC1 HERC6 SLC1A2 RETN | 0.866 | 0.856 | 0.876 | 0.904 | 0.842 | 0.965 | 0.951 | 0.901 | 1 |
| IFI27 IFIT1 IFI44L SIGLEC1 SLC1A2 MMP8 | 0.866 | 0.857 | 0.875 | 0.902 | 0.842 | 0.961 | 0.927 | 0.858 | 0.996 |
| OAS1 SIGLEC1 ISG15 HERC6 RETN MMP8 | 0.866 | 0.856 | 0.876 | 0.897 | 0.829 | 0.964 | 0.952 | 0.905 | 0.999 |
| RSAD2 OAS1 IFI44L SIGLEC1 SLC1A2 IL1R2 | 0.866 | 0.857 | 0.875 | 0.907 | 0.851 | 0.964 | 0.968 | 0.932 | 1 |
| RSAD2 IFI44L HERC6 OLAH FAM20A MMP8 | 0.866 | 0.856 | 0.876 | 0.901 | 0.839 | 0.964 | 0.945 | 0.882 | 1 |
| RSAD2 OAS1 SLC1A2 OLAH FAM20A RETN | 0.866 | 0.856 | 0.876 | 0.910 | 0.852 | 0.968 | 0.935 | 0.862 | 1 |
| RSAD2 IFIT1 IFI44L SIGLEC1 IL1R2 RETN | 0.866 | 0.857 | 0.875 | 0.897 | 0.831 | 0.964 | 0.961 | 0.922 | 1.000 |
| OAS1 IFI44L ISG15 HERC6 OLAH FAM20A | 0.866 | 0.856 | 0.876 | 0.900 | 0.836 | 0.963 | 0.947 | 0.885 | 1 |
| IFI44L ISG15 IL1R2 FAM20A RETN MMP8 | 0.866 | 0.857 | 0.875 | 0.903 | 0.842 | 0.964 | 0.937 | 0.859 | 1 |
| IFI27 OAS1 IFIT1 IFI44L HERC6 MMP8 | 0.866 | 0.857 | 0.875 | 0.900 | 0.842 | 0.957 | 0.897 | 0.817 | 0.976 |
| OAS1 IFI44L SIGLEC1 ISG15 SLC1A2 FAM20A | 0.866 | 0.856 | 0.876 | 0.902 | 0.839 | 0.965 | 0.948 | 0.878 | 1 |
| RSAD2 OAS1 ISG15 IL1R2 FAM20A RETN | 0.866 | 0.856 | 0.876 | 0.898 | 0.833 | 0.964 | 0.935 | 0.856 | 1 |
| IFI27 IFI44L SIGLEC1 HERC6 SLC1A2 MMP8 | 0.866 | 0.857 | 0.875 | 0.902 | 0.845 | 0.960 | 0.924 | 0.854 | 0.993 |
| RSAD2 IFIT1 IFI44L SLC1A2 IL1R2 FAM20A | 0.866 | 0.857 | 0.875 | 0.907 | 0.851 | 0.963 | 0.925 | 0.837 | 1 |
| IFI27 OAS1 IFIT1 IFI44L SLC1A2 RETN | 0.866 | 0.856 | 0.876 | 0.903 | 0.839 | 0.966 | 0.945 | 0.875 | 1 |
| RSAD2 OAS1 ISG15 SLC1A2 IL1R2 FAM20A | 0.866 | 0.856 | 0.875 | 0.904 | 0.848 | 0.961 | 0.932 | 0.846 | 1 |
| OAS1 IFIT1 ISG15 SLC1A2 OLAH FAM20A | 0.866 | 0.856 | 0.876 | 0.908 | 0.849 | 0.967 | 0.936 | 0.862 | 1 |
| RSAD2 IFIT1 SIGLEC1 ISG15 IL1R2 OLAH | 0.866 | 0.857 | 0.875 | 0.899 | 0.836 | 0.963 | 0.959 | 0.917 | 1 |
| RSAD2 SIGLEC1 ISG15 IL1R2 RETN MMP8 | 0.866 | 0.856 | 0.876 | 0.900 | 0.837 | 0.964 | 0.971 | 0.938 | 1 |
| RSAD2 IFI44L SIGLEC1 ISG15 IL1R2 OLAH | 0.866 | 0.857 | 0.875 | 0.896 | 0.831 | 0.962 | 0.958 | 0.916 | 0.999 |
| RSAD2 OAS1 IFI44L SIGLEC1 IL1R2 RETN | 0.866 | 0.856 | 0.875 | 0.893 | 0.826 | 0.961 | 0.963 | 0.924 | 1 |
| OAS1 SIGLEC1 ISG15 HERC6 OLAH RETN | 0.866 | 0.856 | 0.876 | 0.898 | 0.832 | 0.964 | 0.959 | 0.916 | 1 |
| IFI27 OAS1 IFI44L ISG15 SLC1A2 RETN | 0.866 | 0.856 | 0.875 | 0.903 | 0.840 | 0.966 | 0.945 | 0.877 | 1 |
| IFI27 OAS1 ISG15 HERC6 SLC1A2 MMP8 | 0.866 | 0.857 | 0.875 | 0.906 | 0.852 | 0.960 | 0.901 | 0.822 | 0.980 |
| RSAD2 IFIT1 HERC6 OLAH FAM20A MMP8 | 0.866 | 0.856 | 0.876 | 0.899 | 0.835 | 0.963 | 0.942 | 0.875 | 1 |
| RSAD2 IFIT1 ISG15 HERC6 FAM20A RETN | 0.866 | 0.856 | 0.876 | 0.900 | 0.837 | 0.963 | 0.935 | 0.858 | 1 |
| RSAD2 OAS1 SIGLEC1 IL1R2 OLAH RETN | 0.866 | 0.856 | 0.876 | 0.898 | 0.832 | 0.963 | 0.961 | 0.919 | 1 |
| RSAD2 IFI44L SIGLEC1 ISG15 FAM20A RETN | 0.866 | 0.856 | 0.876 | 0.897 | 0.827 | 0.967 | 0.952 | 0.893 | 1 |
| OAS1 IFIT1 SIGLEC1 OLAH RETN MMP8 | 0.866 | 0.856 | 0.876 | 0.896 | 0.829 | 0.964 | 0.953 | 0.909 | 0.998 |
| OAS1 ISG15 IL1R2 FAM20A RETN MMP8 | 0.866 | 0.856 | 0.875 | 0.905 | 0.843 | 0.967 | 0.939 | 0.860 | 1 |
| RSAD2 OAS1 IFIT1 SIGLEC1 ISG15 IL1R2 | 0.866 | 0.857 | 0.875 | 0.898 | 0.835 | 0.961 | 0.962 | 0.923 | 1 |
| RSAD2 OAS1 IFIT1 SLC1A2 OLAH FAM20A | 0.866 | 0.856 | 0.875 | 0.909 | 0.849 | 0.968 | 0.934 | 0.859 | 1 |
| RSAD2 IFI27 OAS1 IFI44L SLC1A2 RETN | 0.866 | 0.856 | 0.875 | 0.905 | 0.843 | 0.968 | 0.940 | 0.868 | 1 |
| OAS1 IFI44L IL1R2 OLAH FAM20A RETN | 0.866 | 0.856 | 0.875 | 0.900 | 0.835 | 0.966 | 0.943 | 0.874 | 1 |
| OAS1 IFI44L SIGLEC1 SLC1A2 FAM20A RETN | 0.866 | 0.856 | 0.875 | 0.901 | 0.838 | 0.965 | 0.947 | 0.875 | 1 |
| IFI27 IFI44L ISG15 SLC1A2 RETN MMP8 | 0.865 | 0.855 | 0.876 | 0.904 | 0.841 | 0.966 | 0.939 | 0.870 | 1 |
| OAS1 SIGLEC1 HERC6 SLC1A2 RETN MMP8 | 0.865 | 0.856 | 0.875 | 0.903 | 0.840 | 0.965 | 0.950 | 0.900 | 1.000 |
| RSAD2 IFI44L SIGLEC1 ISG15 FAM20A MMP8 | 0.865 | 0.855 | 0.876 | 0.894 | 0.823 | 0.964 | 0.948 | 0.883 | 1 |
| RSAD2 IFI44L ISG15 IL1R2 OLAH FAM20A | 0.865 | 0.856 | 0.875 | 0.900 | 0.835 | 0.965 | 0.951 | 0.890 | 1 |
| OAS1 IFI44L SIGLEC1 IL1R2 OLAH RETN | 0.865 | 0.856 | 0.875 | 0.899 | 0.834 | 0.964 | 0.958 | 0.916 | 0.999 |
| OAS1 IFI44L HERC6 OLAH FAM20A MMP8 | 0.865 | 0.855 | 0.876 | 0.899 | 0.835 | 0.964 | 0.947 | 0.884 | 1 |
| OAS1 SIGLEC1 ISG15 IL1R2 RETN MMP8 | 0.865 | 0.856 | 0.875 | 0.901 | 0.837 | 0.964 | 0.971 | 0.938 | 1 |
| IFI27 OAS1 IFIT1 SLC1A2 RETN MMP8 | 0.865 | 0.856 | 0.875 | 0.900 | 0.836 | 0.965 | 0.939 | 0.865 | 1 |
| RSAD2 IFI27 OAS1 IFIT1 HERC6 MMP8 | 0.865 | 0.856 | 0.874 | 0.900 | 0.842 | 0.959 | 0.899 | 0.819 | 0.978 |
| IFI27 IFIT1 IFI44L ISG15 HERC6 MMP8 | 0.865 | 0.856 | 0.875 | 0.897 | 0.838 | 0.957 | 0.895 | 0.814 | 0.976 |
| RSAD2 IFIT1 ISG15 HERC6 OLAH FAM20A | 0.865 | 0.855 | 0.875 | 0.900 | 0.837 | 0.962 | 0.938 | 0.868 | 1 |
| RSAD2 ISG15 HERC6 OLAH FAM20A MMP8 | 0.865 | 0.855 | 0.875 | 0.901 | 0.839 | 0.962 | 0.939 | 0.869 | 1 |
| RSAD2 OAS1 SIGLEC1 ISG15 IL1R2 MMP8 | 0.865 | 0.856 | 0.875 | 0.903 | 0.844 | 0.962 | 0.965 | 0.929 | 1 |
| OAS1 IFI44L SIGLEC1 ISG15 HERC6 RETN | 0.865 | 0.855 | 0.875 | 0.896 | 0.828 | 0.964 | 0.951 | 0.903 | 0.999 |
| IFI27 OAS1 IFI44L SLC1A2 FAM20A MMP8 | 0.865 | 0.855 | 0.876 | 0.902 | 0.837 | 0.966 | 0.936 | 0.855 | 1 |
| RSAD2 IFI27 IFIT1 SIGLEC1 SLC1A2 MMP8 | 0.865 | 0.856 | 0.874 | 0.902 | 0.842 | 0.961 | 0.926 | 0.857 | 0.995 |
| RSAD2 IFI27 OAS1 IFI44L RETN MMP8 | 0.865 | 0.855 | 0.875 | 0.905 | 0.841 | 0.968 | 0.946 | 0.881 | 1 |
| RSAD2 IFIT1 SIGLEC1 HERC6 SLC1A2 RETN | 0.865 | 0.856 | 0.874 | 0.903 | 0.843 | 0.963 | 0.929 | 0.871 | 0.987 |
| OAS1 ISG15 SLC1A2 IL1R2 FAM20A MMP8 | 0.865 | 0.856 | 0.875 | 0.912 | 0.860 | 0.965 | 0.926 | 0.835 | 1 |
| RSAD2 IFI27 OAS1 IFIT1 RETN MMP8 | 0.865 | 0.855 | 0.875 | 0.905 | 0.841 | 0.969 | 0.948 | 0.884 | 1 |
| RSAD2 IL1R2 OLAH FAM20A RETN MMP8 | 0.865 | 0.855 | 0.875 | 0.903 | 0.840 | 0.966 | 0.942 | 0.870 | 1 |
| RSAD2 IFI44L HERC6 OLAH FAM20A RETN | 0.865 | 0.855 | 0.875 | 0.904 | 0.841 | 0.967 | 0.938 | 0.871 | 1 |
| RSAD2 IFIT1 ISG15 IL1R2 OLAH FAM20A | 0.865 | 0.856 | 0.874 | 0.899 | 0.833 | 0.965 | 0.952 | 0.890 | 1 |
| RSAD2 IFIT1 IFI44L SIGLEC1 HERC6 OLAH | 0.865 | 0.856 | 0.874 | 0.900 | 0.838 | 0.962 | 0.937 | 0.883 | 0.991 |
| IFIT1 IFI44L IL1R2 FAM20A RETN MMP8 | 0.865 | 0.855 | 0.875 | 0.903 | 0.840 | 0.967 | 0.946 | 0.873 | 1 |
| IFI44L SIGLEC1 ISG15 HERC6 OLAH RETN | 0.865 | 0.855 | 0.875 | 0.894 | 0.824 | 0.964 | 0.953 | 0.909 | 0.998 |
| OAS1 IFIT1 SLC1A2 OLAH FAM20A RETN | 0.865 | 0.855 | 0.875 | 0.909 | 0.851 | 0.968 | 0.935 | 0.861 | 1 |
| RSAD2 IFI44L SIGLEC1 SLC1A2 FAM20A RETN | 0.865 | 0.855 | 0.875 | 0.904 | 0.842 | 0.966 | 0.946 | 0.874 | 1 |
| IFIT1 SLC1A2 OLAH FAM20A RETN MMP8 | 0.865 | 0.855 | 0.875 | 0.906 | 0.846 | 0.966 | 0.941 | 0.872 | 1 |
| OAS1 IFI44L SLC1A2 IL1R2 FAM20A RETN | 0.865 | 0.855 | 0.875 | 0.908 | 0.852 | 0.963 | 0.925 | 0.837 | 1 |
| OAS1 IFI44L ISG15 HERC6 FAM20A RETN | 0.865 | 0.855 | 0.875 | 0.899 | 0.834 | 0.964 | 0.942 | 0.868 | 1 |
| RSAD2 ISG15 SLC1A2 OLAH FAM20A MMP8 | 0.865 | 0.855 | 0.874 | 0.906 | 0.848 | 0.965 | 0.932 | 0.857 | 1 |
| IFI44L ISG15 SLC1A2 IL1R2 OLAH RETN | 0.865 | 0.855 | 0.874 | 0.910 | 0.854 | 0.966 | 0.938 | 0.883 | 0.993 |
| IFI44L ISG15 SLC1A2 IL1R2 OLAH MMP8 | 0.865 | 0.856 | 0.874 | 0.904 | 0.847 | 0.960 | 0.935 | 0.875 | 0.994 |
| IFIT1 IFI44L SIGLEC1 IL1R2 OLAH RETN | 0.865 | 0.856 | 0.874 | 0.899 | 0.834 | 0.963 | 0.958 | 0.914 | 1 |
| RSAD2 IFI44L SIGLEC1 IL1R2 OLAH RETN | 0.865 | 0.855 | 0.874 | 0.897 | 0.831 | 0.964 | 0.962 | 0.922 | 1 |
| RSAD2 IFIT1 IFI44L IL1R2 FAM20A MMP8 | 0.865 | 0.856 | 0.874 | 0.904 | 0.841 | 0.966 | 0.943 | 0.869 | 1 |
| SIGLEC1 ISG15 HERC6 OLAH RETN MMP8 | 0.865 | 0.854 | 0.875 | 0.892 | 0.819 | 0.964 | 0.961 | 0.922 | 1.000 |
| RSAD2 IFIT1 SIGLEC1 ISG15 HERC6 OLAH | 0.865 | 0.855 | 0.875 | 0.901 | 0.839 | 0.962 | 0.940 | 0.888 | 0.992 |
| OAS1 IFI44L SIGLEC1 HERC6 OLAH RETN | 0.865 | 0.855 | 0.875 | 0.896 | 0.829 | 0.963 | 0.950 | 0.903 | 0.997 |
| OAS1 IFI44L SIGLEC1 ISG15 IL1R2 RETN | 0.865 | 0.856 | 0.874 | 0.898 | 0.833 | 0.963 | 0.959 | 0.918 | 0.999 |
| IFI27 IFIT1 IFI44L ISG15 SLC1A2 RETN | 0.865 | 0.855 | 0.875 | 0.902 | 0.838 | 0.966 | 0.945 | 0.878 | 1 |
| IFI27 OAS1 IFI44L HERC6 SLC1A2 MMP8 | 0.865 | 0.856 | 0.874 | 0.904 | 0.849 | 0.959 | 0.907 | 0.831 | 0.982 |
| IFIT1 SIGLEC1 ISG15 IL1R2 OLAH RETN | 0.865 | 0.856 | 0.874 | 0.900 | 0.836 | 0.963 | 0.961 | 0.919 | 1 |
| OAS1 ISG15 SLC1A2 OLAH FAM20A RETN | 0.865 | 0.855 | 0.875 | 0.912 | 0.855 | 0.969 | 0.937 | 0.865 | 1 |
| OAS1 IFI44L IL1R2 FAM20A RETN MMP8 | 0.865 | 0.855 | 0.874 | 0.907 | 0.845 | 0.969 | 0.943 | 0.868 | 1 |
| RSAD2 OAS1 SLC1A2 IL1R2 FAM20A RETN | 0.865 | 0.856 | 0.874 | 0.905 | 0.849 | 0.962 | 0.930 | 0.844 | 1 |
| RSAD2 IFI44L SIGLEC1 ISG15 IL1R2 RETN | 0.865 | 0.856 | 0.874 | 0.895 | 0.828 | 0.962 | 0.959 | 0.918 | 0.999 |
| OAS1 ISG15 HERC6 OLAH FAM20A MMP8 | 0.865 | 0.855 | 0.875 | 0.898 | 0.834 | 0.962 | 0.946 | 0.879 | 1 |
| IFI44L SLC1A2 IL1R2 OLAH RETN MMP8 | 0.865 | 0.855 | 0.874 | 0.906 | 0.849 | 0.963 | 0.942 | 0.890 | 0.995 |
| OAS1 IFIT1 ISG15 HERC6 IL1R2 RETN | 0.865 | 0.855 | 0.875 | 0.896 | 0.833 | 0.960 | 0.949 | 0.899 | 0.998 |
| OAS1 IFIT1 IFI44L IL1R2 FAM20A RETN | 0.865 | 0.855 | 0.874 | 0.900 | 0.835 | 0.965 | 0.939 | 0.863 | 1 |
| RSAD2 IFI27 OAS1 IFIT1 SLC1A2 RETN | 0.865 | 0.855 | 0.874 | 0.901 | 0.836 | 0.966 | 0.941 | 0.866 | 1 |
| IFIT1 ISG15 SLC1A2 OLAH FAM20A RETN | 0.865 | 0.854 | 0.875 | 0.902 | 0.840 | 0.964 | 0.938 | 0.868 | 1 |
| RSAD2 IFIT1 SIGLEC1 IL1R2 OLAH RETN | 0.865 | 0.855 | 0.874 | 0.898 | 0.832 | 0.964 | 0.959 | 0.916 | 1 |
| IFI27 OAS1 SIGLEC1 ISG15 HERC6 SLC1A2 | 0.865 | 0.856 | 0.874 | 0.900 | 0.844 | 0.956 | 0.951 | 0.900 | 1 |
| RSAD2 OAS1 SIGLEC1 HERC6 OLAH RETN | 0.865 | 0.855 | 0.875 | 0.895 | 0.827 | 0.962 | 0.950 | 0.903 | 0.997 |
| IFI27 SIGLEC1 ISG15 HERC6 SLC1A2 MMP8 | 0.865 | 0.855 | 0.874 | 0.903 | 0.845 | 0.961 | 0.925 | 0.857 | 0.993 |
| RSAD2 SLC1A2 OLAH FAM20A RETN MMP8 | 0.865 | 0.855 | 0.874 | 0.906 | 0.847 | 0.966 | 0.941 | 0.871 | 1 |
| RSAD2 IFI27 SIGLEC1 ISG15 SLC1A2 MMP8 | 0.865 | 0.855 | 0.874 | 0.903 | 0.844 | 0.961 | 0.930 | 0.863 | 0.997 |
| RSAD2 SIGLEC1 ISG15 HERC6 OLAH RETN | 0.864 | 0.855 | 0.874 | 0.896 | 0.830 | 0.963 | 0.954 | 0.910 | 0.998 |
| RSAD2 IFIT1 SIGLEC1 ISG15 IL1R2 MMP8 | 0.864 | 0.856 | 0.873 | 0.901 | 0.841 | 0.961 | 0.966 | 0.931 | 1 |
| IFI27 OAS1 IFI44L SLC1A2 RETN MMP8 | 0.864 | 0.855 | 0.874 | 0.903 | 0.840 | 0.966 | 0.936 | 0.866 | 1 |
| IFIT1 ISG15 IL1R2 OLAH FAM20A RETN | 0.864 | 0.854 | 0.875 | 0.898 | 0.834 | 0.962 | 0.952 | 0.891 | 1 |
| RSAD2 OAS1 IFI44L IL1R2 FAM20A RETN | 0.864 | 0.854 | 0.874 | 0.900 | 0.836 | 0.965 | 0.938 | 0.862 | 1 |
| IFI27 IFIT1 ISG15 HERC6 SLC1A2 FAM20A | 0.864 | 0.854 | 0.875 | 0.905 | 0.846 | 0.964 | 0.933 | 0.850 | 1 |
| RSAD2 IFI27 OAS1 IFI44L SLC1A2 FAM20A | 0.864 | 0.853 | 0.875 | 0.903 | 0.839 | 0.967 | 0.935 | 0.855 | 1 |
| IFIT1 SIGLEC1 ISG15 HERC6 SLC1A2 RETN | 0.864 | 0.855 | 0.874 | 0.902 | 0.842 | 0.962 | 0.938 | 0.884 | 0.992 |
| IFI27 OAS1 IFIT1 HERC6 SLC1A2 MMP8 | 0.864 | 0.855 | 0.873 | 0.905 | 0.850 | 0.959 | 0.902 | 0.823 | 0.981 |
| OAS1 SLC1A2 IL1R2 FAM20A RETN MMP8 | 0.864 | 0.855 | 0.874 | 0.912 | 0.857 | 0.966 | 0.929 | 0.841 | 1 |
| RSAD2 IFIT1 SLC1A2 IL1R2 FAM20A RETN | 0.864 | 0.855 | 0.873 | 0.907 | 0.851 | 0.964 | 0.935 | 0.853 | 1 |
| RSAD2 IFI44L SLC1A2 IL1R2 FAM20A RETN | 0.864 | 0.855 | 0.874 | 0.907 | 0.852 | 0.962 | 0.927 | 0.839 | 1 |
| OAS1 ISG15 HERC6 SLC1A2 FAM20A RETN | 0.864 | 0.854 | 0.875 | 0.902 | 0.840 | 0.964 | 0.936 | 0.855 | 1 |
| RSAD2 IFI27 OAS1 ISG15 RETN MMP8 | 0.864 | 0.854 | 0.874 | 0.901 | 0.837 | 0.966 | 0.952 | 0.894 | 1 |
| RSAD2 ISG15 SLC1A2 IL1R2 FAM20A RETN | 0.864 | 0.855 | 0.874 | 0.903 | 0.846 | 0.959 | 0.929 | 0.840 | 1 |
| OAS1 IFI44L SIGLEC1 HERC6 SLC1A2 RETN | 0.864 | 0.855 | 0.874 | 0.904 | 0.842 | 0.965 | 0.951 | 0.901 | 1 |
| RSAD2 ISG15 SLC1A2 IL1R2 FAM20A MMP8 | 0.864 | 0.855 | 0.873 | 0.907 | 0.853 | 0.961 | 0.926 | 0.832 | 1 |
| OAS1 IFI44L SIGLEC1 ISG15 FAM20A MMP8 | 0.864 | 0.853 | 0.875 | 0.891 | 0.818 | 0.963 | 0.955 | 0.893 | 1 |
| RSAD2 IFI27 OAS1 HERC6 SLC1A2 FAM20A | 0.864 | 0.854 | 0.875 | 0.904 | 0.846 | 0.963 | 0.932 | 0.848 | 1 |
| RSAD2 IFI27 IFIT1 IFI44L RETN MMP8 | 0.864 | 0.854 | 0.874 | 0.905 | 0.842 | 0.969 | 0.951 | 0.892 | 1 |
| RSAD2 ISG15 HERC6 SLC1A2 IL1R2 OLAH | 0.864 | 0.855 | 0.873 | 0.905 | 0.848 | 0.962 | 0.943 | 0.889 | 0.998 |
| OAS1 ISG15 SLC1A2 IL1R2 FAM20A RETN | 0.864 | 0.854 | 0.874 | 0.905 | 0.849 | 0.962 | 0.927 | 0.841 | 1 |
| OAS1 SLC1A2 OLAH FAM20A RETN MMP8 | 0.864 | 0.854 | 0.874 | 0.907 | 0.848 | 0.967 | 0.938 | 0.864 | 1 |
| RSAD2 IFI27 IFIT1 IFI44L SLC1A2 FAM20A | 0.864 | 0.853 | 0.875 | 0.901 | 0.836 | 0.966 | 0.940 | 0.861 | 1 |
| IFIT1 SIGLEC1 HERC6 SLC1A2 RETN MMP8 | 0.864 | 0.855 | 0.873 | 0.904 | 0.844 | 0.963 | 0.932 | 0.875 | 0.988 |
| RSAD2 OAS1 SIGLEC1 HERC6 OLAH MMP8 | 0.864 | 0.854 | 0.874 | 0.896 | 0.833 | 0.959 | 0.949 | 0.902 | 0.996 |
| OAS1 ISG15 HERC6 IL1R2 RETN MMP8 | 0.864 | 0.854 | 0.874 | 0.898 | 0.836 | 0.961 | 0.948 | 0.895 | 1 |
| IFIT1 IFI44L HERC6 SLC1A2 OLAH RETN | 0.864 | 0.854 | 0.874 | 0.907 | 0.849 | 0.965 | 0.934 | 0.875 | 0.993 |
| IFI27 IFIT1 ISG15 SLC1A2 RETN MMP8 | 0.864 | 0.854 | 0.874 | 0.903 | 0.841 | 0.966 | 0.943 | 0.876 | 1 |
| OAS1 SIGLEC1 ISG15 IL1R2 OLAH RETN | 0.864 | 0.854 | 0.874 | 0.900 | 0.836 | 0.965 | 0.959 | 0.917 | 1 |
| IFI27 OAS1 IFIT1 SIGLEC1 HERC6 SLC1A2 | 0.864 | 0.855 | 0.873 | 0.901 | 0.846 | 0.957 | 0.948 | 0.897 | 0.999 |
| IFI44L ISG15 HERC6 SLC1A2 OLAH MMP8 | 0.864 | 0.854 | 0.874 | 0.904 | 0.846 | 0.962 | 0.939 | 0.885 | 0.994 |
| RSAD2 IFI27 OAS1 ISG15 SLC1A2 RETN | 0.864 | 0.854 | 0.873 | 0.901 | 0.836 | 0.965 | 0.937 | 0.861 | 1 |
| RSAD2 OAS1 IFIT1 SIGLEC1 OLAH RETN | 0.864 | 0.854 | 0.873 | 0.895 | 0.826 | 0.964 | 0.946 | 0.893 | 0.998 |
| OAS1 IFIT1 IFI44L SIGLEC1 OLAH MMP8 | 0.864 | 0.854 | 0.873 | 0.893 | 0.825 | 0.961 | 0.946 | 0.895 | 0.996 |
| RSAD2 SIGLEC1 ISG15 HERC6 SLC1A2 RETN | 0.864 | 0.854 | 0.873 | 0.904 | 0.844 | 0.965 | 0.948 | 0.898 | 0.998 |
| RSAD2 IFI27 IFIT1 ISG15 SLC1A2 RETN | 0.864 | 0.854 | 0.873 | 0.900 | 0.836 | 0.964 | 0.945 | 0.876 | 1 |
| OAS1 IFI44L ISG15 IL1R2 FAM20A RETN | 0.864 | 0.854 | 0.874 | 0.900 | 0.836 | 0.965 | 0.941 | 0.866 | 1 |
| IFIT1 IFI44L ISG15 IL1R2 FAM20A RETN | 0.864 | 0.854 | 0.873 | 0.900 | 0.837 | 0.963 | 0.945 | 0.878 | 1 |
| IFI44L SIGLEC1 ISG15 HERC6 OLAH MMP8 | 0.864 | 0.854 | 0.873 | 0.894 | 0.828 | 0.960 | 0.952 | 0.907 | 0.997 |
| RSAD2 IFI27 IFIT1 HERC6 SLC1A2 FAM20A | 0.864 | 0.853 | 0.874 | 0.906 | 0.848 | 0.964 | 0.933 | 0.850 | 1 |
| IFIT1 IFI44L HERC6 SLC1A2 OLAH MMP8 | 0.864 | 0.854 | 0.874 | 0.905 | 0.848 | 0.963 | 0.940 | 0.886 | 0.994 |
| IFIT1 IFI44L SIGLEC1 HERC6 SLC1A2 RETN | 0.864 | 0.855 | 0.873 | 0.902 | 0.842 | 0.962 | 0.929 | 0.871 | 0.987 |
| OAS1 IFIT1 IFI44L SIGLEC1 OLAH RETN | 0.864 | 0.854 | 0.873 | 0.894 | 0.825 | 0.964 | 0.938 | 0.882 | 0.994 |
| RSAD2 IFI27 IFI44L ISG15 RETN MMP8 | 0.864 | 0.853 | 0.874 | 0.904 | 0.841 | 0.968 | 0.953 | 0.897 | 1 |
| IFIT1 IFI44L ISG15 SLC1A2 OLAH MMP8 | 0.863 | 0.854 | 0.873 | 0.905 | 0.847 | 0.963 | 0.938 | 0.884 | 0.992 |
| RSAD2 OAS1 SIGLEC1 ISG15 IL1R2 RETN | 0.863 | 0.854 | 0.873 | 0.897 | 0.831 | 0.962 | 0.967 | 0.930 | 1 |
| RSAD2 IFIT1 IL1R2 FAM20A RETN MMP8 | 0.863 | 0.854 | 0.873 | 0.904 | 0.840 | 0.968 | 0.945 | 0.872 | 1 |
| RSAD2 ISG15 SLC1A2 OLAH FAM20A RETN | 0.863 | 0.854 | 0.873 | 0.908 | 0.849 | 0.967 | 0.930 | 0.853 | 1 |
| RSAD2 OAS1 ISG15 HERC6 IL1R2 RETN | 0.863 | 0.854 | 0.873 | 0.896 | 0.833 | 0.959 | 0.948 | 0.896 | 0.999 |
| IFI27 OAS1 IFIT1 HERC6 SLC1A2 FAM20A | 0.863 | 0.853 | 0.874 | 0.905 | 0.847 | 0.963 | 0.933 | 0.850 | 1 |
| IFI44L ISG15 SLC1A2 OLAH RETN MMP8 | 0.863 | 0.854 | 0.873 | 0.907 | 0.847 | 0.967 | 0.943 | 0.893 | 0.994 |
| RSAD2 ISG15 HERC6 SLC1A2 FAM20A RETN | 0.863 | 0.853 | 0.874 | 0.902 | 0.840 | 0.964 | 0.937 | 0.858 | 1 |
| IFI44L HERC6 SLC1A2 IL1R2 OLAH MMP8 | 0.863 | 0.854 | 0.873 | 0.908 | 0.852 | 0.963 | 0.943 | 0.891 | 0.996 |
| RSAD2 IFI27 IFI44L ISG15 SLC1A2 RETN | 0.863 | 0.854 | 0.873 | 0.904 | 0.840 | 0.967 | 0.946 | 0.881 | 1 |
| RSAD2 IFI27 IFI44L ISG15 HERC6 MMP8 | 0.863 | 0.854 | 0.872 | 0.896 | 0.838 | 0.955 | 0.896 | 0.817 | 0.975 |
| OAS1 IFI44L SIGLEC1 HERC6 OLAH MMP8 | 0.863 | 0.854 | 0.873 | 0.895 | 0.831 | 0.958 | 0.950 | 0.904 | 0.996 |
| RSAD2 ISG15 IL1R2 OLAH FAM20A RETN | 0.863 | 0.853 | 0.873 | 0.898 | 0.832 | 0.964 | 0.943 | 0.873 | 1 |
| RSAD2 IFIT1 SLC1A2 OLAH FAM20A RETN | 0.863 | 0.854 | 0.873 | 0.907 | 0.848 | 0.967 | 0.938 | 0.866 | 1 |
| RSAD2 OAS1 HERC6 SLC1A2 IL1R2 OLAH | 0.863 | 0.854 | 0.873 | 0.904 | 0.846 | 0.961 | 0.941 | 0.884 | 0.999 |
| RSAD2 IFI27 IFIT1 ISG15 RETN MMP8 | 0.863 | 0.853 | 0.873 | 0.903 | 0.839 | 0.966 | 0.953 | 0.895 | 1 |
| IFIT1 IFI44L SIGLEC1 OLAH RETN MMP8 | 0.863 | 0.853 | 0.873 | 0.896 | 0.828 | 0.964 | 0.959 | 0.915 | 1 |
| OAS1 IFIT1 HERC6 SLC1A2 IL1R2 OLAH | 0.863 | 0.853 | 0.873 | 0.903 | 0.846 | 0.961 | 0.943 | 0.891 | 0.996 |
| IFI27 IFIT1 IFI44L ISG15 SLC1A2 FAM20A | 0.863 | 0.853 | 0.874 | 0.899 | 0.834 | 0.965 | 0.941 | 0.863 | 1 |
| RSAD2 HERC6 SLC1A2 IL1R2 OLAH MMP8 | 0.863 | 0.854 | 0.872 | 0.904 | 0.847 | 0.962 | 0.945 | 0.892 | 0.998 |
| RSAD2 IFIT1 ISG15 SLC1A2 OLAH FAM20A | 0.863 | 0.854 | 0.873 | 0.907 | 0.847 | 0.967 | 0.937 | 0.865 | 1 |
| RSAD2 IFIT1 SIGLEC1 ISG15 IL1R2 RETN | 0.863 | 0.854 | 0.872 | 0.897 | 0.831 | 0.963 | 0.960 | 0.919 | 1 |
| IFIT1 HERC6 SLC1A2 IL1R2 OLAH MMP8 | 0.863 | 0.854 | 0.873 | 0.904 | 0.847 | 0.962 | 0.950 | 0.903 | 0.997 |
| OAS1 IFIT1 HERC6 IL1R2 RETN MMP8 | 0.863 | 0.853 | 0.873 | 0.895 | 0.827 | 0.962 | 0.961 | 0.921 | 1 |
| OAS1 IFIT1 SIGLEC1 ISG15 OLAH RETN | 0.863 | 0.853 | 0.873 | 0.895 | 0.825 | 0.964 | 0.942 | 0.889 | 0.995 |
| OAS1 IFI44L HERC6 SLC1A2 OLAH RETN | 0.863 | 0.853 | 0.874 | 0.907 | 0.849 | 0.966 | 0.935 | 0.875 | 0.994 |
| RSAD2 OAS1 IFIT1 SIGLEC1 OLAH MMP8 | 0.863 | 0.854 | 0.873 | 0.893 | 0.825 | 0.961 | 0.946 | 0.895 | 0.996 |
| RSAD2 IFIT1 IFI44L IL1R2 OLAH FAM20A | 0.863 | 0.854 | 0.872 | 0.898 | 0.832 | 0.964 | 0.950 | 0.888 | 1 |
| IFI44L HERC6 SLC1A2 IL1R2 OLAH RETN | 0.863 | 0.853 | 0.873 | 0.907 | 0.850 | 0.964 | 0.941 | 0.886 | 0.997 |
| RSAD2 IFIT1 SIGLEC1 HERC6 RETN MMP8 | 0.863 | 0.853 | 0.873 | 0.894 | 0.824 | 0.964 | 0.924 | 0.863 | 0.984 |
| RSAD2 IFI27 IFI44L SIGLEC1 SLC1A2 MMP8 | 0.863 | 0.854 | 0.872 | 0.901 | 0.841 | 0.960 | 0.929 | 0.861 | 0.998 |
| RSAD2 SLC1A2 IL1R2 FAM20A RETN MMP8 | 0.863 | 0.854 | 0.872 | 0.909 | 0.855 | 0.963 | 0.926 | 0.832 | 1 |
| IFI44L ISG15 HERC6 SLC1A2 IL1R2 OLAH | 0.863 | 0.853 | 0.873 | 0.906 | 0.850 | 0.963 | 0.943 | 0.890 | 0.997 |
| OAS1 IFI44L ISG15 HERC6 IL1R2 RETN | 0.863 | 0.853 | 0.873 | 0.898 | 0.835 | 0.961 | 0.952 | 0.904 | 1 |
| IFI27 OAS1 IFIT1 ISG15 FAM20A MMP8 | 0.863 | 0.852 | 0.874 | 0.895 | 0.827 | 0.963 | 0.937 | 0.857 | 1 |
| RSAD2 IFI27 OAS1 SLC1A2 RETN MMP8 | 0.863 | 0.853 | 0.873 | 0.901 | 0.837 | 0.965 | 0.929 | 0.850 | 1 |
| RSAD2 IFIT1 HERC6 SLC1A2 IL1R2 OLAH | 0.863 | 0.853 | 0.873 | 0.904 | 0.846 | 0.961 | 0.942 | 0.889 | 0.996 |
| RSAD2 IFI27 OAS1 ISG15 FAM20A MMP8 | 0.863 | 0.853 | 0.873 | 0.893 | 0.824 | 0.961 | 0.938 | 0.858 | 1 |
| RSAD2 ISG15 HERC6 FAM20A RETN MMP8 | 0.863 | 0.853 | 0.873 | 0.904 | 0.842 | 0.967 | 0.936 | 0.859 | 1 |
| OAS1 IFIT1 HERC6 SLC1A2 IL1R2 MMP8 | 0.863 | 0.854 | 0.872 | 0.903 | 0.846 | 0.959 | 0.951 | 0.903 | 1.000 |
| RSAD2 IFI27 OAS1 IFIT1 FAM20A MMP8 | 0.863 | 0.853 | 0.873 | 0.896 | 0.829 | 0.963 | 0.936 | 0.856 | 1 |
| RSAD2 IFI27 OAS1 SIGLEC1 HERC6 SLC1A2 | 0.863 | 0.854 | 0.872 | 0.901 | 0.845 | 0.956 | 0.949 | 0.898 | 0.999 |
| IFIT1 IFI44L ISG15 HERC6 SLC1A2 OLAH | 0.863 | 0.853 | 0.873 | 0.906 | 0.849 | 0.963 | 0.938 | 0.883 | 0.993 |
| RSAD2 OAS1 SIGLEC1 ISG15 HERC6 OLAH | 0.863 | 0.853 | 0.873 | 0.896 | 0.833 | 0.959 | 0.950 | 0.904 | 0.996 |
| RSAD2 IFIT1 IFI44L ISG15 IL1R2 FAM20A | 0.863 | 0.854 | 0.872 | 0.898 | 0.833 | 0.962 | 0.948 | 0.880 | 1 |
| IFI44L HERC6 SLC1A2 OLAH RETN MMP8 | 0.863 | 0.853 | 0.873 | 0.906 | 0.847 | 0.965 | 0.945 | 0.895 | 0.995 |
| IFIT1 ISG15 HERC6 SLC1A2 IL1R2 OLAH | 0.863 | 0.854 | 0.872 | 0.904 | 0.846 | 0.961 | 0.942 | 0.889 | 0.996 |
| OAS1 IFIT1 IFI44L HERC6 FAM20A RETN | 0.863 | 0.853 | 0.873 | 0.895 | 0.827 | 0.963 | 0.930 | 0.851 | 1 |
| IFI27 IFIT1 IFI44L SLC1A2 RETN MMP8 | 0.863 | 0.853 | 0.873 | 0.902 | 0.839 | 0.966 | 0.942 | 0.875 | 1 |
| IFI44L ISG15 HERC6 SLC1A2 OLAH RETN | 0.863 | 0.853 | 0.873 | 0.905 | 0.846 | 0.965 | 0.943 | 0.891 | 0.996 |
| IFI27 OAS1 IFIT1 IFI44L ISG15 FAM20A | 0.863 | 0.852 | 0.873 | 0.895 | 0.826 | 0.964 | 0.940 | 0.861 | 1 |
| IFI44L ISG15 HERC6 SLC1A2 FAM20A RETN | 0.863 | 0.852 | 0.873 | 0.898 | 0.832 | 0.963 | 0.946 | 0.868 | 1 |
| RSAD2 IFI44L ISG15 IL1R2 FAM20A MMP8 | 0.863 | 0.853 | 0.872 | 0.903 | 0.842 | 0.963 | 0.946 | 0.868 | 1 |
| IFI27 OAS1 ISG15 SLC1A2 RETN MMP8 | 0.863 | 0.853 | 0.873 | 0.900 | 0.838 | 0.963 | 0.935 | 0.862 | 1 |
| RSAD2 IFI44L SIGLEC1 ISG15 HERC6 RETN | 0.863 | 0.853 | 0.872 | 0.896 | 0.828 | 0.965 | 0.940 | 0.888 | 0.992 |
| OAS1 ISG15 HERC6 SLC1A2 IL1R2 OLAH | 0.863 | 0.853 | 0.872 | 0.905 | 0.849 | 0.962 | 0.941 | 0.887 | 0.995 |
| OAS1 IFIT1 IFI44L SIGLEC1 ISG15 OLAH | 0.863 | 0.853 | 0.872 | 0.894 | 0.826 | 0.962 | 0.942 | 0.889 | 0.996 |
| RSAD2 SIGLEC1 ISG15 IL1R2 OLAH RETN | 0.863 | 0.853 | 0.872 | 0.897 | 0.832 | 0.963 | 0.963 | 0.923 | 1 |
| RSAD2 SIGLEC1 ISG15 HERC6 RETN MMP8 | 0.863 | 0.852 | 0.873 | 0.896 | 0.828 | 0.965 | 0.945 | 0.895 | 0.994 |
| OAS1 IFIT1 ISG15 HERC6 SLC1A2 IL1R2 | 0.862 | 0.853 | 0.872 | 0.902 | 0.846 | 0.959 | 0.951 | 0.902 | 1 |
| RSAD2 IFI44L HERC6 SLC1A2 IL1R2 OLAH | 0.862 | 0.852 | 0.873 | 0.907 | 0.851 | 0.963 | 0.941 | 0.886 | 0.996 |
| OAS1 IFIT1 IFI44L HERC6 SLC1A2 OLAH | 0.862 | 0.852 | 0.873 | 0.905 | 0.848 | 0.962 | 0.934 | 0.875 | 0.992 |
| IFIT1 IFI44L SLC1A2 IL1R2 OLAH MMP8 | 0.862 | 0.853 | 0.872 | 0.904 | 0.847 | 0.961 | 0.949 | 0.901 | 0.997 |
| RSAD2 IFI27 IFIT1 ISG15 HERC6 MMP8 | 0.862 | 0.854 | 0.871 | 0.898 | 0.839 | 0.956 | 0.893 | 0.813 | 0.974 |
| IFIT1 IFI44L ISG15 HERC6 IL1R2 RETN | 0.862 | 0.853 | 0.872 | 0.896 | 0.832 | 0.960 | 0.949 | 0.900 | 0.998 |
| OAS1 IFIT1 SLC1A2 IL1R2 OLAH MMP8 | 0.862 | 0.853 | 0.872 | 0.903 | 0.845 | 0.960 | 0.950 | 0.903 | 0.997 |
| IFI27 OAS1 IFI44L ISG15 SLC1A2 FAM20A | 0.862 | 0.852 | 0.873 | 0.898 | 0.833 | 0.964 | 0.942 | 0.865 | 1 |
| IFIT1 ISG15 SLC1A2 IL1R2 OLAH MMP8 | 0.862 | 0.854 | 0.871 | 0.902 | 0.844 | 0.960 | 0.949 | 0.902 | 0.996 |
| OAS1 ISG15 HERC6 SLC1A2 OLAH RETN | 0.862 | 0.852 | 0.873 | 0.904 | 0.846 | 0.963 | 0.940 | 0.880 | 1 |
| OAS1 HERC6 SLC1A2 IL1R2 OLAH MMP8 | 0.862 | 0.853 | 0.872 | 0.905 | 0.848 | 0.962 | 0.939 | 0.883 | 0.995 |
| IFIT1 ISG15 HERC6 SLC1A2 FAM20A RETN | 0.862 | 0.852 | 0.873 | 0.901 | 0.838 | 0.964 | 0.939 | 0.860 | 1 |
| IFIT1 IFI44L ISG15 SLC1A2 OLAH RETN | 0.862 | 0.853 | 0.872 | 0.908 | 0.850 | 0.966 | 0.934 | 0.877 | 0.990 |
| RSAD2 OAS1 IFI44L HERC6 SLC1A2 OLAH | 0.862 | 0.852 | 0.872 | 0.905 | 0.848 | 0.962 | 0.938 | 0.883 | 0.994 |
| RSAD2 IFIT1 ISG15 HERC6 IL1R2 RETN | 0.862 | 0.853 | 0.872 | 0.897 | 0.833 | 0.961 | 0.949 | 0.900 | 0.998 |
| RSAD2 IFI44L SIGLEC1 HERC6 OLAH RETN | 0.862 | 0.853 | 0.872 | 0.896 | 0.829 | 0.963 | 0.943 | 0.893 | 0.994 |
| IFIT1 ISG15 HERC6 FAM20A RETN MMP8 | 0.862 | 0.852 | 0.872 | 0.901 | 0.837 | 0.966 | 0.938 | 0.862 | 1 |
| RSAD2 OAS1 SIGLEC1 OLAH RETN MMP8 | 0.862 | 0.852 | 0.872 | 0.892 | 0.822 | 0.962 | 0.959 | 0.917 | 1.000 |
| IFI27 IFIT1 IFI44L SIGLEC1 ISG15 MMP8 | 0.862 | 0.853 | 0.872 | 0.890 | 0.824 | 0.957 | 0.911 | 0.836 | 0.986 |
| ISG15 HERC6 IL1R2 OLAH RETN MMP8 | 0.862 | 0.852 | 0.872 | 0.900 | 0.837 | 0.964 | 0.961 | 0.922 | 1 |
| RSAD2 OAS1 OLAH FAM20A RETN MMP8 | 0.862 | 0.852 | 0.872 | 0.895 | 0.826 | 0.964 | 0.952 | 0.892 | 1 |
| RSAD2 OAS1 IFI44L SIGLEC1 HERC6 RETN | 0.862 | 0.852 | 0.872 | 0.889 | 0.817 | 0.961 | 0.945 | 0.895 | 0.995 |
| RSAD2 IFIT1 ISG15 IL1R2 FAM20A RETN | 0.862 | 0.852 | 0.872 | 0.898 | 0.832 | 0.963 | 0.947 | 0.878 | 1 |
| OAS1 IFI44L ISG15 HERC6 SLC1A2 OLAH | 0.862 | 0.852 | 0.872 | 0.906 | 0.849 | 0.963 | 0.937 | 0.881 | 0.993 |
| RSAD2 OAS1 IFIT1 OLAH FAM20A MMP8 | 0.862 | 0.852 | 0.872 | 0.893 | 0.823 | 0.963 | 0.951 | 0.890 | 1 |
| RSAD2 OAS1 IFI44L OLAH FAM20A MMP8 | 0.862 | 0.852 | 0.873 | 0.897 | 0.829 | 0.965 | 0.947 | 0.888 | 1 |
| RSAD2 IFI44L HERC6 SLC1A2 OLAH RETN | 0.862 | 0.852 | 0.872 | 0.908 | 0.850 | 0.966 | 0.934 | 0.876 | 0.992 |
| OAS1 HERC6 SLC1A2 OLAH RETN MMP8 | 0.862 | 0.852 | 0.872 | 0.902 | 0.843 | 0.962 | 0.941 | 0.886 | 0.997 |
| RSAD2 IFI27 IFIT1 IFI44L SLC1A2 RETN | 0.862 | 0.852 | 0.872 | 0.901 | 0.837 | 0.965 | 0.942 | 0.873 | 1 |
| OAS1 IFIT1 ISG15 SLC1A2 IL1R2 OLAH | 0.862 | 0.852 | 0.872 | 0.908 | 0.851 | 0.965 | 0.943 | 0.892 | 0.995 |
| OAS1 IFI44L HERC6 SLC1A2 OLAH MMP8 | 0.862 | 0.852 | 0.872 | 0.905 | 0.848 | 0.962 | 0.939 | 0.884 | 0.994 |
| RSAD2 SIGLEC1 ISG15 HERC6 OLAH MMP8 | 0.862 | 0.852 | 0.872 | 0.897 | 0.834 | 0.960 | 0.949 | 0.902 | 0.996 |
| RSAD2 OAS1 HERC6 SLC1A2 OLAH RETN | 0.862 | 0.852 | 0.872 | 0.903 | 0.843 | 0.962 | 0.939 | 0.877 | 1 |
| IFIT1 IFI44L ISG15 SLC1A2 IL1R2 OLAH | 0.862 | 0.853 | 0.871 | 0.910 | 0.853 | 0.966 | 0.946 | 0.896 | 0.996 |
| RSAD2 ISG15 HERC6 IL1R2 RETN MMP8 | 0.862 | 0.852 | 0.872 | 0.897 | 0.834 | 0.960 | 0.948 | 0.895 | 1 |
| IFI44L SIGLEC1 ISG15 OLAH RETN MMP8 | 0.862 | 0.852 | 0.872 | 0.892 | 0.822 | 0.962 | 0.962 | 0.921 | 1 |
| OAS1 IFI44L SIGLEC1 ISG15 HERC6 OLAH | 0.862 | 0.852 | 0.872 | 0.896 | 0.833 | 0.959 | 0.953 | 0.909 | 0.998 |
| OAS1 ISG15 HERC6 SLC1A2 IL1R2 RETN | 0.862 | 0.852 | 0.872 | 0.902 | 0.844 | 0.959 | 0.930 | 0.859 | 1 |
| IFIT1 IFI44L SLC1A2 OLAH RETN MMP8 | 0.862 | 0.852 | 0.872 | 0.905 | 0.845 | 0.966 | 0.947 | 0.898 | 0.995 |
| RSAD2 ISG15 IL1R2 FAM20A RETN MMP8 | 0.862 | 0.852 | 0.871 | 0.901 | 0.838 | 0.964 | 0.936 | 0.854 | 1 |
| IFIT1 SIGLEC1 ISG15 OLAH RETN MMP8 | 0.862 | 0.852 | 0.872 | 0.895 | 0.827 | 0.962 | 0.962 | 0.920 | 1 |
| RSAD2 IFIT1 SLC1A2 IL1R2 OLAH MMP8 | 0.862 | 0.853 | 0.871 | 0.903 | 0.846 | 0.960 | 0.951 | 0.905 | 0.997 |
| RSAD2 IFI27 IFI44L SLC1A2 RETN MMP8 | 0.862 | 0.852 | 0.872 | 0.902 | 0.839 | 0.965 | 0.935 | 0.863 | 1 |
| RSAD2 SIGLEC1 HERC6 SLC1A2 RETN MMP8 | 0.862 | 0.852 | 0.871 | 0.903 | 0.841 | 0.965 | 0.940 | 0.887 | 0.993 |
| RSAD2 IFIT1 ISG15 HERC6 SLC1A2 IL1R2 | 0.862 | 0.853 | 0.871 | 0.903 | 0.846 | 0.959 | 0.951 | 0.902 | 1 |
| OAS1 IFIT1 SIGLEC1 ISG15 OLAH MMP8 | 0.862 | 0.852 | 0.871 | 0.893 | 0.825 | 0.961 | 0.943 | 0.892 | 0.995 |
| IFI27 OAS1 IFIT1 SIGLEC1 ISG15 MMP8 | 0.862 | 0.852 | 0.871 | 0.889 | 0.821 | 0.956 | 0.913 | 0.837 | 0.989 |
| RSAD2 HERC6 SLC1A2 IL1R2 OLAH RETN | 0.862 | 0.852 | 0.872 | 0.905 | 0.848 | 0.963 | 0.937 | 0.877 | 0.997 |
| IFIT1 ISG15 HERC6 IL1R2 RETN MMP8 | 0.862 | 0.852 | 0.872 | 0.896 | 0.830 | 0.961 | 0.959 | 0.917 | 1 |
| RSAD2 OAS1 IFIT1 HERC6 IL1R2 MMP8 | 0.862 | 0.853 | 0.871 | 0.897 | 0.835 | 0.959 | 0.949 | 0.902 | 0.996 |
| IFIT1 IFI44L IL1R2 OLAH FAM20A RETN | 0.862 | 0.852 | 0.871 | 0.899 | 0.835 | 0.964 | 0.950 | 0.892 | 1 |
| IFI44L HERC6 IL1R2 OLAH RETN MMP8 | 0.862 | 0.852 | 0.872 | 0.900 | 0.836 | 0.963 | 0.960 | 0.920 | 0.999 |
| IFIT1 IFI44L SLC1A2 IL1R2 OLAH RETN | 0.862 | 0.852 | 0.871 | 0.910 | 0.854 | 0.966 | 0.945 | 0.894 | 0.995 |
| RSAD2 OAS1 IFIT1 ISG15 HERC6 IL1R2 | 0.862 | 0.852 | 0.871 | 0.897 | 0.838 | 0.956 | 0.947 | 0.897 | 0.997 |
| RSAD2 IFI44L ISG15 HERC6 SLC1A2 OLAH | 0.862 | 0.852 | 0.872 | 0.906 | 0.849 | 0.963 | 0.934 | 0.877 | 0.990 |
| IFI44L ISG15 HERC6 IL1R2 RETN MMP8 | 0.862 | 0.852 | 0.871 | 0.897 | 0.833 | 0.962 | 0.955 | 0.910 | 1 |
| OAS1 IFIT1 ISG15 HERC6 IL1R2 MMP8 | 0.862 | 0.852 | 0.871 | 0.898 | 0.840 | 0.957 | 0.949 | 0.900 | 0.997 |
| RSAD2 ISG15 HERC6 IL1R2 OLAH RETN | 0.862 | 0.852 | 0.872 | 0.899 | 0.836 | 0.962 | 0.952 | 0.907 | 0.998 |
| RSAD2 OAS1 SIGLEC1 HERC6 RETN MMP8 | 0.862 | 0.852 | 0.872 | 0.892 | 0.820 | 0.963 | 0.943 | 0.893 | 0.994 |
| IFI44L ISG15 HERC6 IL1R2 OLAH RETN | 0.862 | 0.852 | 0.871 | 0.900 | 0.836 | 0.964 | 0.951 | 0.905 | 0.997 |
| OAS1 ISG15 HERC6 IL1R2 OLAH RETN | 0.862 | 0.851 | 0.872 | 0.900 | 0.837 | 0.962 | 0.948 | 0.899 | 0.997 |
| RSAD2 IFIT1 IFI44L HERC6 SLC1A2 OLAH | 0.862 | 0.852 | 0.871 | 0.905 | 0.848 | 0.962 | 0.934 | 0.876 | 0.991 |
| RSAD2 IFI27 IFIT1 ISG15 FAM20A MMP8 | 0.862 | 0.851 | 0.872 | 0.895 | 0.828 | 0.962 | 0.935 | 0.854 | 1 |
| OAS1 IFIT1 IFI44L HERC6 IL1R2 RETN | 0.862 | 0.852 | 0.871 | 0.895 | 0.828 | 0.961 | 0.948 | 0.900 | 0.996 |
| RSAD2 IFI44L ISG15 HERC6 IL1R2 RETN | 0.862 | 0.852 | 0.871 | 0.900 | 0.837 | 0.962 | 0.952 | 0.905 | 0.999 |
| OAS1 SIGLEC1 ISG15 HERC6 OLAH MMP8 | 0.862 | 0.852 | 0.871 | 0.892 | 0.827 | 0.956 | 0.955 | 0.912 | 0.999 |
| RSAD2 IFIT1 SIGLEC1 OLAH RETN MMP8 | 0.862 | 0.852 | 0.871 | 0.896 | 0.828 | 0.964 | 0.959 | 0.915 | 1 |
| OAS1 IFIT1 IFI44L HERC6 IL1R2 MMP8 | 0.862 | 0.852 | 0.871 | 0.895 | 0.832 | 0.958 | 0.949 | 0.902 | 0.996 |
| IFI27 OAS1 IFIT1 IFI44L SIGLEC1 MMP8 | 0.861 | 0.852 | 0.871 | 0.889 | 0.823 | 0.956 | 0.916 | 0.842 | 0.990 |
| IFIT1 HERC6 SLC1A2 OLAH RETN MMP8 | 0.861 | 0.851 | 0.872 | 0.903 | 0.843 | 0.963 | 0.950 | 0.902 | 0.998 |
| RSAD2 ISG15 HERC6 SLC1A2 OLAH RETN | 0.861 | 0.852 | 0.871 | 0.904 | 0.845 | 0.963 | 0.943 | 0.888 | 0.999 |
| RSAD2 IFI27 IFIT1 IFI44L HERC6 MMP8 | 0.861 | 0.852 | 0.871 | 0.897 | 0.838 | 0.956 | 0.892 | 0.812 | 0.973 |
| RSAD2 HERC6 SLC1A2 OLAH RETN MMP8 | 0.861 | 0.852 | 0.871 | 0.903 | 0.843 | 0.963 | 0.943 | 0.890 | 0.997 |
| OAS1 IFI44L HERC6 SLC1A2 IL1R2 OLAH | 0.861 | 0.851 | 0.872 | 0.907 | 0.851 | 0.963 | 0.939 | 0.882 | 0.996 |
| RSAD2 IFI44L HERC6 SLC1A2 OLAH MMP8 | 0.861 | 0.851 | 0.871 | 0.905 | 0.848 | 0.962 | 0.932 | 0.873 | 0.990 |
| IFIT1 IFI44L SIGLEC1 ISG15 OLAH MMP8 | 0.861 | 0.852 | 0.871 | 0.891 | 0.824 | 0.958 | 0.946 | 0.894 | 0.998 |
| IFI27 IFIT1 IFI44L HERC6 SLC1A2 MMP8 | 0.861 | 0.852 | 0.871 | 0.900 | 0.842 | 0.957 | 0.902 | 0.820 | 0.984 |
| ISG15 HERC6 SLC1A2 IL1R2 OLAH RETN | 0.861 | 0.852 | 0.871 | 0.907 | 0.851 | 0.964 | 0.941 | 0.884 | 0.999 |
| RSAD2 OAS1 IFIT1 HERC6 SLC1A2 IL1R2 | 0.861 | 0.852 | 0.871 | 0.900 | 0.843 | 0.958 | 0.950 | 0.899 | 1 |
| OAS1 HERC6 SLC1A2 IL1R2 OLAH RETN | 0.861 | 0.851 | 0.871 | 0.904 | 0.846 | 0.961 | 0.933 | 0.869 | 0.996 |
| RSAD2 IFI27 IFIT1 SLC1A2 RETN MMP8 | 0.861 | 0.852 | 0.871 | 0.902 | 0.839 | 0.965 | 0.942 | 0.874 | 1 |
| IFI27 OAS1 IFI44L SIGLEC1 HERC6 SLC1A2 | 0.861 | 0.853 | 0.870 | 0.901 | 0.846 | 0.957 | 0.947 | 0.895 | 0.999 |
| OAS1 IFIT1 HERC6 SLC1A2 OLAH MMP8 | 0.861 | 0.851 | 0.872 | 0.902 | 0.844 | 0.960 | 0.946 | 0.894 | 0.997 |
| RSAD2 IFI27 ISG15 SLC1A2 RETN MMP8 | 0.861 | 0.851 | 0.871 | 0.901 | 0.839 | 0.964 | 0.934 | 0.860 | 1 |
| IFIT1 HERC6 IL1R2 OLAH RETN MMP8 | 0.861 | 0.851 | 0.871 | 0.899 | 0.835 | 0.963 | 0.962 | 0.924 | 1 |
| OAS1 IFIT1 IFI44L SLC1A2 OLAH MMP8 | 0.861 | 0.851 | 0.871 | 0.905 | 0.847 | 0.963 | 0.938 | 0.884 | 0.992 |
| RSAD2 OAS1 IFIT1 HERC6 IL1R2 RETN | 0.861 | 0.852 | 0.871 | 0.894 | 0.827 | 0.961 | 0.947 | 0.898 | 0.996 |
| IFIT1 HERC6 SLC1A2 IL1R2 OLAH RETN | 0.861 | 0.851 | 0.871 | 0.904 | 0.846 | 0.962 | 0.940 | 0.883 | 0.997 |
| RSAD2 OAS1 IFIT1 IFI44L SIGLEC1 OLAH | 0.861 | 0.852 | 0.871 | 0.893 | 0.825 | 0.962 | 0.946 | 0.894 | 0.997 |
| IFIT1 IFI44L ISG15 HERC6 IL1R2 MMP8 | 0.861 | 0.852 | 0.870 | 0.896 | 0.836 | 0.957 | 0.947 | 0.897 | 0.996 |
| RSAD2 OAS1 IFI44L SIGLEC1 HERC6 OLAH | 0.861 | 0.852 | 0.871 | 0.896 | 0.833 | 0.959 | 0.949 | 0.902 | 0.996 |
| RSAD2 IFIT1 IL1R2 OLAH FAM20A RETN | 0.861 | 0.852 | 0.871 | 0.898 | 0.831 | 0.964 | 0.947 | 0.880 | 1 |
| RSAD2 OAS1 ISG15 OLAH FAM20A MMP8 | 0.861 | 0.851 | 0.871 | 0.893 | 0.824 | 0.963 | 0.947 | 0.882 | 1 |
| ISG15 HERC6 SLC1A2 OLAH RETN MMP8 | 0.861 | 0.851 | 0.871 | 0.905 | 0.846 | 0.964 | 0.952 | 0.905 | 0.999 |
| IFIT1 SLC1A2 IL1R2 OLAH RETN MMP8 | 0.861 | 0.852 | 0.871 | 0.906 | 0.849 | 0.964 | 0.957 | 0.914 | 0.999 |
| OAS1 HERC6 IL1R2 OLAH RETN MMP8 | 0.861 | 0.851 | 0.871 | 0.899 | 0.835 | 0.963 | 0.958 | 0.916 | 0.999 |
| IFIT1 ISG15 SLC1A2 IL1R2 OLAH RETN | 0.861 | 0.852 | 0.871 | 0.907 | 0.849 | 0.965 | 0.948 | 0.899 | 0.997 |
| ISG15 HERC6 SLC1A2 IL1R2 OLAH MMP8 | 0.861 | 0.852 | 0.870 | 0.904 | 0.848 | 0.961 | 0.940 | 0.885 | 0.996 |
| RSAD2 OAS1 IFI44L ISG15 OLAH FAM20A | 0.861 | 0.851 | 0.872 | 0.894 | 0.823 | 0.965 | 0.945 | 0.885 | 1 |
| RSAD2 IFIT1 HERC6 SLC1A2 IL1R2 MMP8 | 0.861 | 0.852 | 0.870 | 0.903 | 0.846 | 0.960 | 0.950 | 0.901 | 0.999 |
| IFI27 OAS1 IFIT1 IFI44L SLC1A2 FAM20A | 0.861 | 0.850 | 0.872 | 0.899 | 0.833 | 0.964 | 0.942 | 0.865 | 1 |
| OAS1 ISG15 HERC6 FAM20A RETN MMP8 | 0.861 | 0.851 | 0.871 | 0.899 | 0.834 | 0.964 | 0.939 | 0.863 | 1 |
| RSAD2 OAS1 HERC6 IL1R2 OLAH MMP8 | 0.861 | 0.852 | 0.871 | 0.898 | 0.838 | 0.958 | 0.946 | 0.895 | 0.997 |
| IFIT1 IFI44L HERC6 SLC1A2 IL1R2 OLAH | 0.861 | 0.851 | 0.871 | 0.907 | 0.850 | 0.963 | 0.943 | 0.890 | 0.997 |
| OAS1 IFIT1 HERC6 SLC1A2 IL1R2 RETN | 0.861 | 0.851 | 0.871 | 0.901 | 0.843 | 0.960 | 0.939 | 0.880 | 0.999 |
| IFIT1 ISG15 HERC6 SLC1A2 OLAH MMP8 | 0.861 | 0.851 | 0.871 | 0.902 | 0.844 | 0.960 | 0.943 | 0.891 | 0.996 |
| IFI27 IFIT1 ISG15 SLC1A2 FAM20A MMP8 | 0.861 | 0.850 | 0.872 | 0.896 | 0.831 | 0.961 | 0.933 | 0.851 | 1 |
| RSAD2 OAS1 HERC6 SLC1A2 OLAH MMP8 | 0.861 | 0.851 | 0.871 | 0.902 | 0.844 | 0.960 | 0.942 | 0.888 | 0.997 |
| RSAD2 OAS1 IFIT1 ISG15 OLAH FAM20A | 0.861 | 0.851 | 0.871 | 0.893 | 0.822 | 0.964 | 0.949 | 0.886 | 1 |
| IFI27 IFIT1 ISG15 HERC6 SLC1A2 MMP8 | 0.861 | 0.852 | 0.870 | 0.899 | 0.842 | 0.957 | 0.899 | 0.815 | 0.982 |
| RSAD2 IFI44L SIGLEC1 HERC6 OLAH MMP8 | 0.861 | 0.851 | 0.871 | 0.897 | 0.833 | 0.960 | 0.949 | 0.902 | 0.996 |
| IFIT1 IFI44L HERC6 SLC1A2 FAM20A RETN | 0.861 | 0.851 | 0.871 | 0.900 | 0.838 | 0.963 | 0.926 | 0.844 | 1 |
| OAS1 IFIT1 ISG15 HERC6 SLC1A2 OLAH | 0.861 | 0.851 | 0.871 | 0.903 | 0.845 | 0.960 | 0.945 | 0.893 | 0.996 |
| RSAD2 IFIT1 HERC6 IL1R2 RETN MMP8 | 0.861 | 0.851 | 0.871 | 0.894 | 0.826 | 0.963 | 0.959 | 0.918 | 1.000 |
| RSAD2 IFI27 IFI44L HERC6 SLC1A2 MMP8 | 0.861 | 0.852 | 0.870 | 0.899 | 0.842 | 0.957 | 0.902 | 0.823 | 0.982 |
| IFIT1 IFI44L HERC6 FAM20A RETN MMP8 | 0.861 | 0.851 | 0.871 | 0.898 | 0.830 | 0.966 | 0.932 | 0.852 | 1 |
| IFIT1 IFI44L HERC6 IL1R2 RETN MMP8 | 0.861 | 0.851 | 0.871 | 0.894 | 0.826 | 0.962 | 0.959 | 0.918 | 1.000 |
| OAS1 IFI44L SIGLEC1 OLAH RETN MMP8 | 0.861 | 0.851 | 0.871 | 0.892 | 0.822 | 0.963 | 0.965 | 0.928 | 1 |
| OAS1 IFIT1 HERC6 SLC1A2 OLAH RETN | 0.861 | 0.851 | 0.871 | 0.901 | 0.841 | 0.960 | 0.939 | 0.880 | 0.998 |
| RSAD2 OAS1 HERC6 IL1R2 OLAH RETN | 0.861 | 0.851 | 0.871 | 0.898 | 0.834 | 0.962 | 0.947 | 0.897 | 0.996 |
| OAS1 IFIT1 SLC1A2 IL1R2 OLAH RETN | 0.861 | 0.851 | 0.871 | 0.908 | 0.850 | 0.965 | 0.946 | 0.895 | 0.996 |
| RSAD2 OAS1 IFIT1 SLC1A2 IL1R2 OLAH | 0.861 | 0.851 | 0.871 | 0.905 | 0.847 | 0.963 | 0.948 | 0.899 | 0.997 |
| IFIT1 ISG15 HERC6 SLC1A2 IL1R2 MMP8 | 0.861 | 0.851 | 0.870 | 0.902 | 0.845 | 0.959 | 0.953 | 0.906 | 1 |
| RSAD2 OAS1 IFIT1 SIGLEC1 ISG15 OLAH | 0.861 | 0.851 | 0.870 | 0.894 | 0.826 | 0.962 | 0.945 | 0.893 | 0.996 |
| OAS1 ISG15 HERC6 SLC1A2 OLAH MMP8 | 0.861 | 0.851 | 0.871 | 0.902 | 0.845 | 0.960 | 0.940 | 0.886 | 0.995 |
| OAS1 ISG15 HERC6 OLAH RETN MMP8 | 0.861 | 0.850 | 0.871 | 0.895 | 0.828 | 0.962 | 0.961 | 0.921 | 1 |
| RSAD2 IFIT1 ISG15 HERC6 IL1R2 MMP8 | 0.861 | 0.851 | 0.870 | 0.897 | 0.837 | 0.957 | 0.947 | 0.897 | 0.996 |
| RSAD2 IFI44L SIGLEC1 HERC6 SLC1A2 RETN | 0.861 | 0.852 | 0.870 | 0.902 | 0.840 | 0.964 | 0.939 | 0.886 | 0.993 |
| HERC6 SLC1A2 IL1R2 OLAH RETN MMP8 | 0.861 | 0.851 | 0.870 | 0.904 | 0.846 | 0.962 | 0.939 | 0.881 | 0.997 |
| OAS1 IFIT1 ISG15 HERC6 OLAH RETN | 0.861 | 0.851 | 0.871 | 0.896 | 0.831 | 0.961 | 0.951 | 0.905 | 0.997 |
| IFIT1 ISG15 HERC6 IL1R2 OLAH RETN | 0.861 | 0.851 | 0.870 | 0.900 | 0.836 | 0.963 | 0.951 | 0.905 | 0.997 |
| RSAD2 IFIT1 IFI44L HERC6 FAM20A RETN | 0.861 | 0.851 | 0.871 | 0.898 | 0.832 | 0.964 | 0.925 | 0.844 | 1 |
| RSAD2 IFI44L ISG15 HERC6 SLC1A2 FAM20A | 0.861 | 0.850 | 0.871 | 0.899 | 0.839 | 0.959 | 0.925 | 0.842 | 1 |
| RSAD2 IFIT1 IFI44L SLC1A2 OLAH MMP8 | 0.861 | 0.851 | 0.870 | 0.905 | 0.847 | 0.964 | 0.937 | 0.883 | 0.991 |
| RSAD2 OAS1 HERC6 IL1R2 RETN MMP8 | 0.861 | 0.851 | 0.870 | 0.894 | 0.828 | 0.961 | 0.943 | 0.889 | 0.998 |
| OAS1 IFIT1 IFI44L OLAH FAM20A MMP8 | 0.861 | 0.850 | 0.871 | 0.893 | 0.822 | 0.963 | 0.951 | 0.895 | 1 |
| IFI27 OAS1 IFI44L SIGLEC1 ISG15 MMP8 | 0.860 | 0.851 | 0.870 | 0.890 | 0.824 | 0.957 | 0.917 | 0.845 | 0.990 |
| RSAD2 OAS1 IFIT1 HERC6 IL1R2 OLAH | 0.860 | 0.851 | 0.870 | 0.899 | 0.837 | 0.960 | 0.946 | 0.896 | 0.995 |
| RSAD2 OAS1 IFIT1 SLC1A2 OLAH MMP8 | 0.860 | 0.851 | 0.870 | 0.904 | 0.846 | 0.962 | 0.946 | 0.897 | 0.995 |
| OAS1 IFIT1 ISG15 OLAH FAM20A RETN | 0.860 | 0.850 | 0.871 | 0.893 | 0.822 | 0.964 | 0.952 | 0.894 | 1 |
| RSAD2 OAS1 IFI44L SLC1A2 OLAH MMP8 | 0.860 | 0.851 | 0.870 | 0.905 | 0.848 | 0.963 | 0.935 | 0.879 | 0.991 |
| RSAD2 HERC6 IL1R2 OLAH RETN MMP8 | 0.860 | 0.850 | 0.871 | 0.899 | 0.835 | 0.962 | 0.959 | 0.918 | 0.999 |
| OAS1 IFIT1 HERC6 IL1R2 OLAH MMP8 | 0.860 | 0.851 | 0.870 | 0.899 | 0.838 | 0.959 | 0.954 | 0.911 | 0.998 |
| RSAD2 OAS1 IFIT1 IFI44L HERC6 IL1R2 | 0.860 | 0.851 | 0.870 | 0.895 | 0.833 | 0.958 | 0.946 | 0.896 | 0.995 |
| IFIT1 ISG15 HERC6 SLC1A2 OLAH RETN | 0.860 | 0.850 | 0.870 | 0.904 | 0.845 | 0.963 | 0.943 | 0.888 | 0.999 |
| ISG15 SLC1A2 IL1R2 FAM20A RETN MMP8 | 0.860 | 0.851 | 0.869 | 0.906 | 0.851 | 0.967 | 0.937 | 0.882 | 0.991 |
| OAS1 IFIT1 IFI44L SLC1A2 OLAH RETN | 0.860 | 0.850 | 0.871 | 0.909 | 0.851 | 0.967 | 0.937 | 0.882 | 0.991 |
| RSAD2 IFIT1 HERC6 SLC1A2 OLAH MMP8 | 0.860 | 0.851 | 0.870 | 0.901 | 0.843 | 0.960 | 0.943 | 0.891 | 0.996 |
| IFIT1 HERC6 SLC1A2 IL1R2 RETN MMP8 | 0.860 | 0.851 | 0.870 | 0.903 | 0.845 | 0.962 | 0.950 | 0.899 | 1 |
| RSAD2 IFI44L IL1R2 OLAH FAM20A RETN | 0.860 | 0.850 | 0.870 | 0.897 | 0.832 | 0.963 | 0.949 | 0.885 | 1 |
| IFI27 OAS1 IFIT1 SIGLEC1 ISG15 SLC1A2 | 0.860 | 0.851 | 0.869 | 0.897 | 0.837 | 0.956 | 0.954 | 0.905 | 1 |
| RSAD2 OAS1 IFIT1 IFI44L OLAH FAM20A | 0.860 | 0.851 | 0.870 | 0.894 | 0.824 | 0.964 | 0.943 | 0.885 | 1 |
| RSAD2 OAS1 ISG15 HERC6 SLC1A2 OLAH | 0.860 | 0.850 | 0.870 | 0.902 | 0.845 | 0.959 | 0.942 | 0.889 | 0.996 |
| OAS1 IFI44L HERC6 IL1R2 RETN MMP8 | 0.860 | 0.851 | 0.870 | 0.895 | 0.829 | 0.961 | 0.950 | 0.903 | 0.997 |
| RSAD2 IFI27 OAS1 IFIT1 ISG15 FAM20A | 0.860 | 0.850 | 0.871 | 0.888 | 0.817 | 0.959 | 0.941 | 0.862 | 1 |
| ISG15 IL1R2 OLAH FAM20A RETN MMP8 | 0.860 | 0.851 | 0.870 | 0.901 | 0.842 | 0.961 | 0.935 | 0.864 | 1 |
| RSAD2 OAS1 IFI44L HERC6 FAM20A RETN | 0.860 | 0.850 | 0.870 | 0.895 | 0.828 | 0.962 | 0.927 | 0.847 | 1 |
| RSAD2 OAS1 IFIT1 HERC6 SLC1A2 OLAH | 0.860 | 0.850 | 0.870 | 0.903 | 0.845 | 0.960 | 0.949 | 0.898 | 1.000 |
| IFI44L ISG15 HERC6 IL1R2 OLAH MMP8 | 0.860 | 0.851 | 0.870 | 0.899 | 0.840 | 0.958 | 0.953 | 0.909 | 0.998 |
| RSAD2 IFI27 OAS1 IFI44L SIGLEC1 MMP8 | 0.860 | 0.851 | 0.870 | 0.890 | 0.823 | 0.957 | 0.924 | 0.854 | 0.994 |
| IFIT1 IFI44L ISG15 HERC6 OLAH RETN | 0.860 | 0.851 | 0.870 | 0.895 | 0.827 | 0.962 | 0.942 | 0.891 | 0.994 |
| RSAD2 OAS1 ISG15 OLAH FAM20A RETN | 0.860 | 0.850 | 0.870 | 0.894 | 0.823 | 0.965 | 0.951 | 0.889 | 1 |
| RSAD2 IFI27 ISG15 HERC6 SLC1A2 MMP8 | 0.860 | 0.851 | 0.869 | 0.901 | 0.845 | 0.957 | 0.902 | 0.823 | 0.982 |
| IFIT1 ISG15 HERC6 IL1R2 OLAH MMP8 | 0.860 | 0.851 | 0.870 | 0.900 | 0.840 | 0.959 | 0.959 | 0.918 | 1.000 |
| OAS1 IFIT1 IFI44L ISG15 SLC1A2 OLAH | 0.860 | 0.850 | 0.870 | 0.909 | 0.852 | 0.966 | 0.936 | 0.881 | 0.990 |
| OAS1 ISG15 HERC6 IL1R2 OLAH MMP8 | 0.860 | 0.850 | 0.870 | 0.900 | 0.841 | 0.959 | 0.947 | 0.898 | 0.995 |
| RSAD2 IFIT1 SIGLEC1 ISG15 OLAH RETN | 0.860 | 0.850 | 0.870 | 0.895 | 0.826 | 0.964 | 0.949 | 0.897 | 1 |
| IFIT1 IFI44L SIGLEC1 ISG15 OLAH RETN | 0.860 | 0.851 | 0.870 | 0.891 | 0.822 | 0.960 | 0.941 | 0.884 | 0.998 |
| IFIT1 IFI44L HERC6 OLAH RETN MMP8 | 0.860 | 0.850 | 0.870 | 0.892 | 0.823 | 0.962 | 0.953 | 0.909 | 0.997 |
| RSAD2 OAS1 ISG15 HERC6 IL1R2 OLAH | 0.860 | 0.850 | 0.870 | 0.898 | 0.838 | 0.959 | 0.945 | 0.895 | 0.995 |
| IFI44L ISG15 HERC6 SLC1A2 IL1R2 RETN | 0.860 | 0.850 | 0.870 | 0.906 | 0.849 | 0.962 | 0.942 | 0.882 | 1 |
| OAS1 IFIT1 IFI44L ISG15 HERC6 IL1R2 | 0.860 | 0.851 | 0.869 | 0.897 | 0.838 | 0.956 | 0.947 | 0.897 | 0.997 |
| ISG15 HERC6 SLC1A2 FAM20A RETN MMP8 | 0.860 | 0.850 | 0.870 | 0.900 | 0.836 | 0.965 | 0.940 | 0.862 | 1 |
| IFIT1 ISG15 HERC6 SLC1A2 IL1R2 RETN | 0.860 | 0.850 | 0.870 | 0.905 | 0.848 | 0.962 | 0.943 | 0.887 | 1 |
| RSAD2 IFI44L SLC1A2 IL1R2 OLAH MMP8 | 0.860 | 0.851 | 0.869 | 0.904 | 0.848 | 0.961 | 0.942 | 0.888 | 0.997 |
| IFIT1 IFI44L HERC6 SLC1A2 IL1R2 MMP8 | 0.860 | 0.851 | 0.869 | 0.903 | 0.846 | 0.960 | 0.951 | 0.902 | 1.000 |
| RSAD2 IFI44L ISG15 IL1R2 FAM20A RETN | 0.860 | 0.850 | 0.870 | 0.897 | 0.833 | 0.962 | 0.942 | 0.871 | 1 |
| RSAD2 IFIT1 HERC6 SLC1A2 OLAH RETN | 0.860 | 0.850 | 0.870 | 0.904 | 0.845 | 0.963 | 0.937 | 0.877 | 0.997 |
| RSAD2 ISG15 HERC6 IL1R2 OLAH MMP8 | 0.860 | 0.850 | 0.869 | 0.899 | 0.840 | 0.958 | 0.949 | 0.902 | 0.996 |
| IFIT1 IFI44L HERC6 IL1R2 OLAH MMP8 | 0.860 | 0.850 | 0.869 | 0.899 | 0.838 | 0.959 | 0.955 | 0.912 | 0.999 |
| RSAD2 IFIT1 IFI44L SIGLEC1 OLAH RETN | 0.860 | 0.850 | 0.869 | 0.895 | 0.825 | 0.964 | 0.948 | 0.896 | 1.000 |
| RSAD2 IFIT1 IFI44L ISG15 HERC6 IL1R2 | 0.860 | 0.851 | 0.869 | 0.898 | 0.838 | 0.957 | 0.947 | 0.897 | 0.997 |
| RSAD2 IFI27 OAS1 SLC1A2 FAM20A MMP8 | 0.860 | 0.849 | 0.870 | 0.895 | 0.830 | 0.960 | 0.928 | 0.844 | 1 |
| OAS1 IFI44L SIGLEC1 HERC6 RETN MMP8 | 0.860 | 0.850 | 0.869 | 0.889 | 0.818 | 0.961 | 0.949 | 0.901 | 0.997 |
| IFI27 OAS1 ISG15 SLC1A2 FAM20A MMP8 | 0.860 | 0.849 | 0.870 | 0.895 | 0.829 | 0.960 | 0.929 | 0.846 | 1 |
| RSAD2 OAS1 ISG15 HERC6 OLAH RETN | 0.860 | 0.850 | 0.870 | 0.895 | 0.830 | 0.960 | 0.953 | 0.909 | 0.998 |
| RSAD2 OAS1 IFI44L HERC6 IL1R2 OLAH | 0.860 | 0.850 | 0.869 | 0.899 | 0.838 | 0.959 | 0.946 | 0.895 | 0.996 |
| OAS1 ISG15 HERC6 SLC1A2 IL1R2 MMP8 | 0.860 | 0.850 | 0.869 | 0.902 | 0.847 | 0.957 | 0.933 | 0.867 | 0.998 |
| RSAD2 OAS1 IFIT1 OLAH FAM20A RETN | 0.860 | 0.850 | 0.870 | 0.894 | 0.823 | 0.964 | 0.948 | 0.885 | 1 |
| RSAD2 ISG15 HERC6 SLC1A2 IL1R2 RETN | 0.860 | 0.850 | 0.869 | 0.902 | 0.844 | 0.959 | 0.940 | 0.878 | 1 |
| RSAD2 IFIT1 ISG15 SLC1A2 IL1R2 OLAH | 0.860 | 0.850 | 0.869 | 0.905 | 0.847 | 0.964 | 0.947 | 0.897 | 0.997 |
| RSAD2 OAS1 IFI44L HERC6 IL1R2 RETN | 0.860 | 0.850 | 0.869 | 0.893 | 0.827 | 0.960 | 0.945 | 0.894 | 0.995 |
| OAS1 IFIT1 HERC6 IL1R2 OLAH RETN | 0.860 | 0.849 | 0.870 | 0.897 | 0.833 | 0.962 | 0.947 | 0.897 | 0.996 |
| RSAD2 OAS1 ISG15 HERC6 IL1R2 MMP8 | 0.860 | 0.850 | 0.869 | 0.898 | 0.839 | 0.956 | 0.946 | 0.895 | 0.996 |
| RSAD2 IFI27 SIGLEC1 ISG15 HERC6 SLC1A2 | 0.860 | 0.851 | 0.868 | 0.898 | 0.841 | 0.954 | 0.942 | 0.889 | 0.996 |
| IFI44L ISG15 HERC6 FAM20A RETN MMP8 | 0.860 | 0.849 | 0.870 | 0.895 | 0.826 | 0.965 | 0.947 | 0.874 | 1 |
| RSAD2 OAS1 SLC1A2 IL1R2 OLAH MMP8 | 0.859 | 0.850 | 0.869 | 0.904 | 0.846 | 0.961 | 0.939 | 0.882 | 0.996 |
| RSAD2 IFIT1 IFI44L HERC6 IL1R2 MMP8 | 0.859 | 0.850 | 0.869 | 0.895 | 0.832 | 0.958 | 0.950 | 0.903 | 0.997 |
| RSAD2 IFIT1 ISG15 HERC6 IL1R2 OLAH | 0.859 | 0.850 | 0.869 | 0.900 | 0.839 | 0.960 | 0.950 | 0.904 | 0.996 |
| RSAD2 IFI27 ISG15 SLC1A2 FAM20A MMP8 | 0.859 | 0.849 | 0.870 | 0.895 | 0.830 | 0.960 | 0.932 | 0.850 | 1 |
| OAS1 IFIT1 ISG15 SLC1A2 OLAH MMP8 | 0.859 | 0.850 | 0.869 | 0.903 | 0.845 | 0.961 | 0.941 | 0.889 | 0.993 |
| OAS1 IFI44L ISG15 HERC6 OLAH RETN | 0.859 | 0.849 | 0.870 | 0.895 | 0.829 | 0.960 | 0.958 | 0.916 | 1.000 |
| OAS1 IFI44L ISG15 SLC1A2 OLAH RETN | 0.859 | 0.849 | 0.869 | 0.909 | 0.851 | 0.967 | 0.935 | 0.879 | 0.990 |
| RSAD2 IFI27 OAS1 IFIT1 SIGLEC1 MMP8 | 0.859 | 0.850 | 0.869 | 0.889 | 0.822 | 0.956 | 0.916 | 0.841 | 0.991 |
| RSAD2 ISG15 HERC6 SLC1A2 OLAH MMP8 | 0.859 | 0.850 | 0.869 | 0.901 | 0.843 | 0.960 | 0.940 | 0.885 | 0.995 |
| IFIT1 ISG15 HERC6 OLAH RETN MMP8 | 0.859 | 0.849 | 0.870 | 0.893 | 0.825 | 0.961 | 0.955 | 0.913 | 0.998 |
| IFI27 OAS1 IFIT1 SLC1A2 FAM20A MMP8 | 0.859 | 0.849 | 0.870 | 0.897 | 0.832 | 0.961 | 0.933 | 0.852 | 1 |
| OAS1 IFI44L ISG15 SLC1A2 OLAH MMP8 | 0.859 | 0.850 | 0.869 | 0.905 | 0.847 | 0.963 | 0.936 | 0.881 | 0.991 |
| RSAD2 IFIT1 IFI44L SIGLEC1 OLAH MMP8 | 0.859 | 0.850 | 0.869 | 0.894 | 0.827 | 0.961 | 0.953 | 0.907 | 0.999 |
| OAS1 IFIT1 ISG15 SLC1A2 OLAH RETN | 0.859 | 0.849 | 0.869 | 0.907 | 0.849 | 0.965 | 0.945 | 0.895 | 0.994 |
| RSAD2 OAS1 IFIT1 IFI44L SLC1A2 OLAH | 0.859 | 0.850 | 0.869 | 0.909 | 0.852 | 0.966 | 0.935 | 0.880 | 0.990 |
| IFIT1 IFI44L ISG15 HERC6 IL1R2 OLAH | 0.859 | 0.850 | 0.869 | 0.899 | 0.838 | 0.959 | 0.949 | 0.902 | 0.996 |
| RSAD2 IFI27 IFIT1 HERC6 SLC1A2 MMP8 | 0.859 | 0.850 | 0.869 | 0.900 | 0.843 | 0.957 | 0.900 | 0.817 | 0.983 |
| RSAD2 ISG15 HERC6 OLAH RETN MMP8 | 0.859 | 0.849 | 0.870 | 0.895 | 0.828 | 0.962 | 0.957 | 0.915 | 0.998 |
| RSAD2 IFI27 OAS1 SIGLEC1 ISG15 HERC6 | 0.859 | 0.850 | 0.869 | 0.895 | 0.840 | 0.951 | 0.921 | 0.858 | 0.984 |
| RSAD2 IFI44L SIGLEC1 ISG15 HERC6 OLAH | 0.859 | 0.850 | 0.869 | 0.899 | 0.836 | 0.961 | 0.950 | 0.904 | 0.996 |
| OAS1 IFI44L HERC6 SLC1A2 IL1R2 RETN | 0.859 | 0.849 | 0.869 | 0.902 | 0.844 | 0.960 | 0.936 | 0.873 | 0.999 |
| RSAD2 OAS1 HERC6 OLAH RETN MMP8 | 0.859 | 0.849 | 0.869 | 0.890 | 0.821 | 0.960 | 0.954 | 0.911 | 0.998 |
| OAS1 IFIT1 ISG15 HERC6 IL1R2 OLAH | 0.859 | 0.849 | 0.869 | 0.899 | 0.838 | 0.959 | 0.952 | 0.907 | 0.997 |
| RSAD2 IFI44L HERC6 SLC1A2 FAM20A RETN | 0.859 | 0.849 | 0.869 | 0.901 | 0.838 | 0.963 | 0.929 | 0.850 | 1 |
| OAS1 IFI44L SLC1A2 OLAH RETN MMP8 | 0.859 | 0.849 | 0.869 | 0.905 | 0.845 | 0.966 | 0.941 | 0.889 | 0.993 |
| RSAD2 IFI27 IFIT1 IFI44L SIGLEC1 MMP8 | 0.859 | 0.850 | 0.869 | 0.891 | 0.825 | 0.957 | 0.914 | 0.840 | 0.989 |
| OAS1 IFIT1 IFI44L HERC6 OLAH RETN | 0.859 | 0.849 | 0.869 | 0.893 | 0.824 | 0.962 | 0.940 | 0.888 | 0.993 |
| RSAD2 IFIT1 ISG15 HERC6 SLC1A2 OLAH | 0.859 | 0.850 | 0.869 | 0.904 | 0.846 | 0.961 | 0.943 | 0.891 | 0.996 |
| RSAD2 ISG15 SLC1A2 IL1R2 OLAH MMP8 | 0.859 | 0.850 | 0.868 | 0.903 | 0.845 | 0.960 | 0.937 | 0.877 | 0.997 |
| OAS1 SIGLEC1 ISG15 OLAH RETN MMP8 | 0.859 | 0.849 | 0.869 | 0.892 | 0.821 | 0.962 | 0.963 | 0.922 | 1 |
| RSAD2 OAS1 IFI44L OLAH FAM20A RETN | 0.859 | 0.849 | 0.869 | 0.892 | 0.821 | 0.963 | 0.945 | 0.885 | 1 |
| OAS1 IFIT1 IFI44L HERC6 SLC1A2 IL1R2 | 0.859 | 0.849 | 0.869 | 0.903 | 0.847 | 0.960 | 0.949 | 0.899 | 0.999 |
| RSAD2 IFIT1 IFI44L HERC6 IL1R2 RETN | 0.859 | 0.850 | 0.869 | 0.893 | 0.826 | 0.960 | 0.947 | 0.898 | 0.995 |
| RSAD2 IFI27 IFIT1 SLC1A2 FAM20A MMP8 | 0.859 | 0.849 | 0.869 | 0.896 | 0.832 | 0.961 | 0.933 | 0.851 | 1 |
| RSAD2 IFI44L ISG15 SLC1A2 IL1R2 OLAH | 0.859 | 0.850 | 0.869 | 0.906 | 0.849 | 0.963 | 0.941 | 0.887 | 0.995 |
| OAS1 IFIT1 IFI44L SLC1A2 IL1R2 OLAH | 0.859 | 0.849 | 0.869 | 0.910 | 0.854 | 0.966 | 0.945 | 0.893 | 0.996 |
| OAS1 IFI44L ISG15 SLC1A2 IL1R2 OLAH | 0.859 | 0.849 | 0.869 | 0.907 | 0.850 | 0.964 | 0.940 | 0.885 | 0.995 |
| RSAD2 OAS1 ISG15 HERC6 SLC1A2 IL1R2 | 0.859 | 0.849 | 0.869 | 0.900 | 0.844 | 0.956 | 0.942 | 0.879 | 1 |
| OAS1 IFI44L HERC6 IL1R2 OLAH RETN | 0.859 | 0.849 | 0.869 | 0.898 | 0.834 | 0.963 | 0.947 | 0.897 | 0.996 |
| IFIT1 IFI44L ISG15 HERC6 SLC1A2 IL1R2 | 0.859 | 0.850 | 0.868 | 0.902 | 0.845 | 0.959 | 0.950 | 0.900 | 1.000 |
| OAS1 IFIT1 SIGLEC1 HERC6 SLC1A2 MMP8 | 0.859 | 0.850 | 0.868 | 0.892 | 0.831 | 0.953 | 0.913 | 0.845 | 0.981 |
| RSAD2 OAS1 IFI44L SLC1A2 OLAH RETN | 0.859 | 0.849 | 0.869 | 0.909 | 0.851 | 0.967 | 0.936 | 0.881 | 0.991 |
| OAS1 IFIT1 OLAH FAM20A RETN MMP8 | 0.859 | 0.849 | 0.869 | 0.890 | 0.818 | 0.962 | 0.958 | 0.904 | 1 |
| OAS1 IFIT1 HERC6 OLAH RETN MMP8 | 0.859 | 0.849 | 0.869 | 0.891 | 0.822 | 0.960 | 0.955 | 0.913 | 0.998 |
| RSAD2 OAS1 SLC1A2 IL1R2 OLAH RETN | 0.859 | 0.849 | 0.869 | 0.905 | 0.848 | 0.963 | 0.939 | 0.882 | 0.996 |
| RSAD2 IFI44L SLC1A2 IL1R2 OLAH RETN | 0.859 | 0.849 | 0.869 | 0.907 | 0.850 | 0.963 | 0.939 | 0.884 | 0.994 |
| RSAD2 IFIT1 SLC1A2 IL1R2 OLAH RETN | 0.859 | 0.849 | 0.868 | 0.907 | 0.849 | 0.964 | 0.946 | 0.895 | 0.996 |
| ISG15 HERC6 SLC1A2 IL1R2 RETN MMP8 | 0.859 | 0.849 | 0.868 | 0.902 | 0.845 | 0.959 | 0.942 | 0.881 | 1 |
| RSAD2 IFI44L IL1R2 FAM20A RETN MMP8 | 0.859 | 0.849 | 0.869 | 0.902 | 0.839 | 0.964 | 0.940 | 0.862 | 1 |
| RSAD2 IFI44L ISG15 SLC1A2 OLAH MMP8 | 0.859 | 0.849 | 0.868 | 0.905 | 0.848 | 0.963 | 0.937 | 0.882 | 0.991 |
| IFI44L HERC6 SLC1A2 IL1R2 RETN MMP8 | 0.859 | 0.849 | 0.869 | 0.902 | 0.844 | 0.960 | 0.943 | 0.885 | 1 |
| RSAD2 OAS1 ISG15 SLC1A2 IL1R2 OLAH | 0.859 | 0.849 | 0.868 | 0.904 | 0.847 | 0.962 | 0.938 | 0.880 | 0.996 |
| IFIT1 IFI44L HERC6 SLC1A2 IL1R2 RETN | 0.859 | 0.849 | 0.868 | 0.901 | 0.842 | 0.960 | 0.939 | 0.881 | 0.998 |
| OAS1 IFIT1 IFI44L HERC6 IL1R2 OLAH | 0.859 | 0.849 | 0.868 | 0.898 | 0.837 | 0.959 | 0.946 | 0.896 | 0.995 |
| RSAD2 IFIT1 HERC6 SLC1A2 IL1R2 RETN | 0.859 | 0.849 | 0.868 | 0.901 | 0.842 | 0.960 | 0.942 | 0.886 | 0.999 |
| RSAD2 IFI44L ISG15 SLC1A2 OLAH RETN | 0.859 | 0.849 | 0.869 | 0.909 | 0.851 | 0.967 | 0.934 | 0.877 | 0.990 |
| RSAD2 OAS1 IFI44L SLC1A2 IL1R2 OLAH | 0.859 | 0.849 | 0.869 | 0.906 | 0.849 | 0.963 | 0.940 | 0.885 | 0.995 |
| OAS1 IFI44L HERC6 SLC1A2 FAM20A RETN | 0.859 | 0.849 | 0.869 | 0.897 | 0.833 | 0.961 | 0.939 | 0.862 | 1 |
| ISG15 SLC1A2 OLAH FAM20A RETN MMP8 | 0.859 | 0.849 | 0.869 | 0.904 | 0.848 | 0.961 | 0.923 | 0.847 | 0.999 |
| IFI44L ISG15 OLAH FAM20A RETN MMP8 | 0.859 | 0.849 | 0.869 | 0.896 | 0.828 | 0.964 | 0.962 | 0.920 | 1 |
| IFI27 IFI44L ISG15 HERC6 SLC1A2 MMP8 | 0.859 | 0.850 | 0.868 | 0.899 | 0.842 | 0.957 | 0.903 | 0.821 | 0.986 |
| IFIT1 IFI44L HERC6 IL1R2 OLAH RETN | 0.859 | 0.849 | 0.868 | 0.898 | 0.833 | 0.963 | 0.948 | 0.899 | 0.996 |
| RSAD2 SLC1A2 IL1R2 OLAH RETN MMP8 | 0.859 | 0.849 | 0.868 | 0.905 | 0.848 | 0.963 | 0.941 | 0.885 | 0.998 |
| OAS1 IFI44L HERC6 IL1R2 OLAH MMP8 | 0.859 | 0.849 | 0.868 | 0.900 | 0.840 | 0.959 | 0.951 | 0.904 | 0.998 |
| RSAD2 IFIT1 IFI44L SLC1A2 OLAH RETN | 0.859 | 0.849 | 0.868 | 0.908 | 0.851 | 0.966 | 0.937 | 0.883 | 0.991 |
| RSAD2 IFI44L SLC1A2 OLAH RETN MMP8 | 0.859 | 0.849 | 0.868 | 0.905 | 0.845 | 0.966 | 0.942 | 0.891 | 0.994 |
| OAS1 IFI44L ISG15 HERC6 IL1R2 MMP8 | 0.859 | 0.849 | 0.868 | 0.896 | 0.838 | 0.954 | 0.945 | 0.894 | 0.995 |
| OAS1 IFI44L SLC1A2 IL1R2 OLAH MMP8 | 0.859 | 0.849 | 0.868 | 0.905 | 0.849 | 0.962 | 0.941 | 0.888 | 0.995 |
| IFI27 OAS1 IFIT1 SIGLEC1 ISG15 HERC6 | 0.859 | 0.849 | 0.868 | 0.898 | 0.843 | 0.953 | 0.930 | 0.873 | 0.988 |
| RSAD2 IFIT1 IFI44L SLC1A2 IL1R2 OLAH | 0.859 | 0.849 | 0.868 | 0.909 | 0.852 | 0.966 | 0.946 | 0.895 | 0.996 |
| RSAD2 OAS1 HERC6 SLC1A2 IL1R2 RETN | 0.859 | 0.849 | 0.868 | 0.899 | 0.840 | 0.958 | 0.934 | 0.865 | 1 |
| RSAD2 OAS1 IFI44L HERC6 IL1R2 MMP8 | 0.859 | 0.849 | 0.868 | 0.894 | 0.833 | 0.955 | 0.942 | 0.891 | 0.994 |
| OAS1 IFI44L SLC1A2 IL1R2 OLAH RETN | 0.859 | 0.848 | 0.869 | 0.908 | 0.851 | 0.965 | 0.938 | 0.883 | 0.993 |
| RSAD2 IFI27 IFI44L SIGLEC1 HERC6 SLC1A2 | 0.858 | 0.850 | 0.867 | 0.899 | 0.843 | 0.954 | 0.940 | 0.885 | 0.995 |
| RSAD2 OAS1 IFIT1 HERC6 FAM20A RETN | 0.858 | 0.848 | 0.869 | 0.887 | 0.816 | 0.958 | 0.934 | 0.855 | 1 |
| RSAD2 IFI44L ISG15 HERC6 SLC1A2 IL1R2 | 0.858 | 0.849 | 0.868 | 0.902 | 0.846 | 0.957 | 0.940 | 0.882 | 0.999 |
| RSAD2 IFI44L ISG15 HERC6 IL1R2 OLAH | 0.858 | 0.849 | 0.868 | 0.900 | 0.841 | 0.959 | 0.946 | 0.896 | 0.995 |
| RSAD2 IFIT1 HERC6 IL1R2 OLAH MMP8 | 0.858 | 0.848 | 0.868 | 0.899 | 0.838 | 0.959 | 0.957 | 0.914 | 0.999 |
| IFI44L ISG15 HERC6 OLAH RETN MMP8 | 0.858 | 0.848 | 0.869 | 0.892 | 0.821 | 0.962 | 0.961 | 0.922 | 1.000 |
| IFI27 OAS1 IFI44L SIGLEC1 ISG15 SLC1A2 | 0.858 | 0.849 | 0.867 | 0.896 | 0.837 | 0.956 | 0.953 | 0.903 | 1 |
| OAS1 IFIT1 ISG15 OLAH FAM20A MMP8 | 0.858 | 0.848 | 0.869 | 0.893 | 0.823 | 0.964 | 0.952 | 0.892 | 1 |
| RSAD2 IFI27 OAS1 SIGLEC1 ISG15 MMP8 | 0.858 | 0.848 | 0.868 | 0.890 | 0.824 | 0.956 | 0.921 | 0.848 | 0.993 |
| OAS1 IFIT1 ISG15 SLC1A2 IL1R2 RETN | 0.858 | 0.849 | 0.868 | 0.899 | 0.838 | 0.959 | 0.949 | 0.900 | 0.998 |
| RSAD2 IFIT1 SIGLEC1 ISG15 OLAH MMP8 | 0.858 | 0.849 | 0.868 | 0.893 | 0.826 | 0.961 | 0.949 | 0.900 | 0.998 |
| RSAD2 ISG15 SLC1A2 IL1R2 OLAH RETN | 0.858 | 0.849 | 0.868 | 0.906 | 0.849 | 0.963 | 0.939 | 0.881 | 0.998 |
| RSAD2 IFI44L ISG15 HERC6 OLAH RETN | 0.858 | 0.848 | 0.868 | 0.897 | 0.832 | 0.962 | 0.947 | 0.899 | 0.995 |
| RSAD2 OAS1 IFI44L ISG15 SLC1A2 OLAH | 0.858 | 0.848 | 0.868 | 0.907 | 0.850 | 0.964 | 0.934 | 0.878 | 0.990 |
| RSAD2 OAS1 SIGLEC1 ISG15 OLAH MMP8 | 0.858 | 0.848 | 0.868 | 0.891 | 0.823 | 0.959 | 0.947 | 0.897 | 0.996 |
| RSAD2 IFI27 IFIT1 SIGLEC1 HERC6 SLC1A2 | 0.858 | 0.849 | 0.867 | 0.900 | 0.845 | 0.955 | 0.942 | 0.889 | 0.995 |
| IFI27 OAS1 IFI44L SIGLEC1 ISG15 HERC6 | 0.858 | 0.849 | 0.867 | 0.897 | 0.842 | 0.953 | 0.932 | 0.874 | 0.989 |
| RSAD2 IFI44L HERC6 IL1R2 RETN MMP8 | 0.858 | 0.848 | 0.868 | 0.894 | 0.827 | 0.961 | 0.950 | 0.902 | 0.998 |
| RSAD2 IFI44L HERC6 IL1R2 OLAH RETN | 0.858 | 0.848 | 0.868 | 0.899 | 0.834 | 0.963 | 0.948 | 0.899 | 0.996 |
| OAS1 IFIT1 IFI44L ISG15 OLAH FAM20A | 0.858 | 0.848 | 0.868 | 0.890 | 0.818 | 0.963 | 0.950 | 0.895 | 1 |
| RSAD2 IFI44L HERC6 IL1R2 OLAH MMP8 | 0.858 | 0.848 | 0.868 | 0.899 | 0.839 | 0.959 | 0.952 | 0.906 | 0.999 |
| RSAD2 IFIT1 ISG15 HERC6 OLAH RETN | 0.858 | 0.848 | 0.868 | 0.894 | 0.828 | 0.960 | 0.942 | 0.892 | 0.993 |
| RSAD2 ISG15 HERC6 SLC1A2 IL1R2 MMP8 | 0.858 | 0.849 | 0.867 | 0.900 | 0.843 | 0.957 | 0.936 | 0.872 | 1.000 |
| RSAD2 IFIT1 IFI44L IL1R2 FAM20A RETN | 0.858 | 0.848 | 0.868 | 0.895 | 0.830 | 0.960 | 0.946 | 0.874 | 1 |
| OAS1 IFIT1 SLC1A2 OLAH RETN MMP8 | 0.858 | 0.848 | 0.868 | 0.899 | 0.838 | 0.961 | 0.954 | 0.911 | 0.998 |
| RSAD2 IFIT1 IFI44L ISG15 SLC1A2 OLAH | 0.858 | 0.849 | 0.867 | 0.907 | 0.850 | 0.965 | 0.935 | 0.880 | 0.990 |
| OAS1 IFI44L ISG15 HERC6 IL1R2 OLAH | 0.858 | 0.848 | 0.868 | 0.900 | 0.841 | 0.959 | 0.948 | 0.900 | 0.996 |
| RSAD2 OAS1 SIGLEC1 ISG15 OLAH RETN | 0.858 | 0.848 | 0.868 | 0.892 | 0.821 | 0.963 | 0.949 | 0.899 | 0.999 |
| RSAD2 OAS1 IFI44L ISG15 HERC6 IL1R2 | 0.858 | 0.848 | 0.867 | 0.897 | 0.840 | 0.955 | 0.947 | 0.896 | 0.997 |
| RSAD2 OAS1 IFI44L SIGLEC1 OLAH MMP8 | 0.858 | 0.848 | 0.868 | 0.893 | 0.826 | 0.961 | 0.948 | 0.899 | 0.997 |
| RSAD2 IFI27 IFI44L SIGLEC1 ISG15 MMP8 | 0.858 | 0.848 | 0.868 | 0.889 | 0.822 | 0.956 | 0.916 | 0.844 | 0.988 |
| RSAD2 OAS1 IFIT1 SLC1A2 IL1R2 MMP8 | 0.858 | 0.849 | 0.867 | 0.902 | 0.844 | 0.960 | 0.954 | 0.909 | 1.000 |
| RSAD2 IFI44L ISG15 HERC6 IL1R2 MMP8 | 0.858 | 0.849 | 0.867 | 0.895 | 0.836 | 0.954 | 0.942 | 0.891 | 0.994 |
| RSAD2 OAS1 IFI44L ISG15 HERC6 FAM20A | 0.858 | 0.848 | 0.868 | 0.890 | 0.824 | 0.956 | 0.922 | 0.841 | 1 |
| RSAD2 IFIT1 IFI44L HERC6 SLC1A2 IL1R2 | 0.858 | 0.849 | 0.867 | 0.902 | 0.844 | 0.960 | 0.947 | 0.896 | 0.998 |
| OAS1 IFI44L ISG15 OLAH FAM20A MMP8 | 0.858 | 0.847 | 0.869 | 0.890 | 0.818 | 0.962 | 0.953 | 0.898 | 1 |
| OAS1 ISG15 SLC1A2 IL1R2 OLAH MMP8 | 0.858 | 0.849 | 0.867 | 0.905 | 0.848 | 0.962 | 0.930 | 0.868 | 0.993 |
| IFI27 OAS1 IFIT1 IFI44L SIGLEC1 SLC1A2 | 0.858 | 0.849 | 0.867 | 0.896 | 0.836 | 0.955 | 0.955 | 0.906 | 1 |
| IFIT1 IFI44L ISG15 OLAH FAM20A MMP8 | 0.858 | 0.848 | 0.868 | 0.889 | 0.818 | 0.960 | 0.958 | 0.911 | 1 |
| OAS1 ISG15 SLC1A2 IL1R2 OLAH RETN | 0.858 | 0.848 | 0.868 | 0.907 | 0.849 | 0.964 | 0.934 | 0.874 | 0.994 |
| RSAD2 IFIT1 IFI44L HERC6 IL1R2 OLAH | 0.858 | 0.848 | 0.867 | 0.899 | 0.838 | 0.960 | 0.946 | 0.896 | 0.995 |
| IFI44L ISG15 HERC6 SLC1A2 IL1R2 MMP8 | 0.858 | 0.849 | 0.867 | 0.901 | 0.846 | 0.957 | 0.939 | 0.880 | 0.998 |
| OAS1 IFI44L HERC6 OLAH RETN MMP8 | 0.858 | 0.847 | 0.868 | 0.891 | 0.822 | 0.960 | 0.954 | 0.911 | 0.998 |
| OAS1 IFIT1 ISG15 SLC1A2 IL1R2 MMP8 | 0.858 | 0.849 | 0.867 | 0.903 | 0.846 | 0.960 | 0.952 | 0.906 | 0.999 |
| RSAD2 OAS1 IFI44L SIGLEC1 OLAH RETN | 0.858 | 0.848 | 0.868 | 0.891 | 0.820 | 0.961 | 0.953 | 0.907 | 0.999 |
| OAS1 IFIT1 IFI44L OLAH FAM20A RETN | 0.858 | 0.848 | 0.868 | 0.889 | 0.817 | 0.962 | 0.949 | 0.893 | 1 |
| OAS1 ISG15 OLAH FAM20A RETN MMP8 | 0.858 | 0.847 | 0.868 | 0.892 | 0.821 | 0.963 | 0.958 | 0.902 | 1 |
| OAS1 IFI44L ISG15 HERC6 SLC1A2 IL1R2 | 0.858 | 0.848 | 0.868 | 0.900 | 0.845 | 0.956 | 0.940 | 0.879 | 1 |
| OAS1 IFIT1 IFI44L ISG15 HERC6 RETN | 0.858 | 0.848 | 0.868 | 0.892 | 0.825 | 0.959 | 0.924 | 0.859 | 0.988 |
| RSAD2 IFIT1 IFI44L SIGLEC1 ISG15 OLAH | 0.858 | 0.848 | 0.867 | 0.895 | 0.828 | 0.962 | 0.947 | 0.895 | 0.999 |
| RSAD2 SIGLEC1 ISG15 OLAH RETN MMP8 | 0.858 | 0.847 | 0.868 | 0.892 | 0.822 | 0.963 | 0.963 | 0.922 | 1 |
| RSAD2 OAS1 IFIT1 SLC1A2 OLAH RETN | 0.858 | 0.848 | 0.867 | 0.906 | 0.847 | 0.964 | 0.945 | 0.894 | 0.995 |
| IFIT1 IFI44L ISG15 SLC1A2 IL1R2 RETN | 0.858 | 0.848 | 0.867 | 0.899 | 0.840 | 0.958 | 0.949 | 0.900 | 0.997 |
| RSAD2 IFI27 IFIT1 SIGLEC1 ISG15 MMP8 | 0.858 | 0.848 | 0.867 | 0.889 | 0.822 | 0.957 | 0.911 | 0.835 | 0.987 |
| OAS1 HERC6 SLC1A2 IL1R2 RETN MMP8 | 0.857 | 0.848 | 0.867 | 0.900 | 0.841 | 0.958 | 0.928 | 0.860 | 0.997 |
| RSAD2 IFI44L SIGLEC1 OLAH RETN MMP8 | 0.857 | 0.847 | 0.868 | 0.891 | 0.821 | 0.961 | 0.962 | 0.921 | 1 |
| IFI44L HERC6 SLC1A2 FAM20A RETN MMP8 | 0.857 | 0.847 | 0.868 | 0.896 | 0.830 | 0.963 | 0.939 | 0.860 | 1 |
| RSAD2 IFIT1 HERC6 IL1R2 OLAH RETN | 0.857 | 0.847 | 0.867 | 0.897 | 0.833 | 0.962 | 0.946 | 0.896 | 0.996 |
| IFIT1 SIGLEC1 ISG15 HERC6 SLC1A2 MMP8 | 0.857 | 0.848 | 0.866 | 0.894 | 0.834 | 0.954 | 0.914 | 0.846 | 0.982 |
| RSAD2 IFI27 OAS1 SIGLEC1 ISG15 SLC1A2 | 0.857 | 0.848 | 0.867 | 0.896 | 0.837 | 0.955 | 0.951 | 0.900 | 1 |
| RSAD2 IFIT1 ISG15 SLC1A2 OLAH MMP8 | 0.857 | 0.848 | 0.867 | 0.903 | 0.844 | 0.961 | 0.945 | 0.895 | 0.994 |
| RSAD2 OAS1 HERC6 SLC1A2 IL1R2 MMP8 | 0.857 | 0.848 | 0.867 | 0.898 | 0.841 | 0.956 | 0.934 | 0.868 | 0.999 |
| IFIT1 IFI44L ISG15 SLC1A2 IL1R2 MMP8 | 0.857 | 0.849 | 0.866 | 0.904 | 0.847 | 0.960 | 0.953 | 0.907 | 0.999 |
| RSAD2 OAS1 ISG15 SLC1A2 OLAH MMP8 | 0.857 | 0.848 | 0.867 | 0.901 | 0.842 | 0.959 | 0.937 | 0.881 | 0.993 |
| OAS1 IFI44L SIGLEC1 ISG15 OLAH RETN | 0.857 | 0.847 | 0.867 | 0.891 | 0.821 | 0.962 | 0.951 | 0.902 | 1.000 |
| RSAD2 OAS1 IFIT1 HERC6 OLAH RETN | 0.857 | 0.847 | 0.867 | 0.890 | 0.821 | 0.959 | 0.940 | 0.888 | 0.993 |
| RSAD2 IFI44L HERC6 SLC1A2 IL1R2 RETN | 0.857 | 0.848 | 0.867 | 0.901 | 0.843 | 0.960 | 0.934 | 0.869 | 0.998 |
| OAS1 SLC1A2 IL1R2 OLAH RETN MMP8 | 0.857 | 0.847 | 0.867 | 0.906 | 0.849 | 0.963 | 0.936 | 0.878 | 0.993 |
| OAS1 IFI44L SIGLEC1 ISG15 OLAH MMP8 | 0.857 | 0.847 | 0.867 | 0.892 | 0.824 | 0.960 | 0.952 | 0.907 | 0.998 |
| IFI27 OAS1 IFIT1 ISG15 SLC1A2 FAM20A | 0.857 | 0.846 | 0.868 | 0.888 | 0.819 | 0.957 | 0.942 | 0.864 | 1 |
| OAS1 IFI44L ISG15 HERC6 SLC1A2 RETN | 0.857 | 0.847 | 0.867 | 0.899 | 0.836 | 0.961 | 0.934 | 0.869 | 0.998 |
| RSAD2 IFI27 OAS1 ISG15 SLC1A2 FAM20A | 0.857 | 0.846 | 0.868 | 0.889 | 0.820 | 0.958 | 0.936 | 0.854 | 1 |
| RSAD2 OAS1 IFIT1 IL1R2 OLAH MMP8 | 0.857 | 0.847 | 0.867 | 0.895 | 0.832 | 0.958 | 0.955 | 0.913 | 0.998 |
| RSAD2 OAS1 IFIT1 ISG15 SLC1A2 OLAH | 0.857 | 0.847 | 0.867 | 0.903 | 0.845 | 0.961 | 0.941 | 0.889 | 0.993 |
| RSAD2 IFIT1 SIGLEC1 HERC6 SLC1A2 MMP8 | 0.857 | 0.848 | 0.866 | 0.894 | 0.833 | 0.954 | 0.911 | 0.842 | 0.980 |
| IFIT1 ISG15 SLC1A2 OLAH RETN MMP8 | 0.857 | 0.847 | 0.867 | 0.902 | 0.842 | 0.962 | 0.954 | 0.911 | 0.998 |
| IFI44L SIGLEC1 ISG15 HERC6 SLC1A2 RETN | 0.857 | 0.847 | 0.866 | 0.894 | 0.828 | 0.960 | 0.955 | 0.909 | 1 |
| RSAD2 OAS1 IFI44L ISG15 HERC6 RETN | 0.857 | 0.846 | 0.867 | 0.893 | 0.825 | 0.961 | 0.938 | 0.882 | 0.994 |
| RSAD2 IFI27 OAS1 IFI44L SIGLEC1 HERC6 | 0.857 | 0.848 | 0.866 | 0.898 | 0.843 | 0.953 | 0.920 | 0.855 | 0.984 |
| RSAD2 OAS1 IFI44L HERC6 SLC1A2 IL1R2 | 0.857 | 0.847 | 0.867 | 0.898 | 0.841 | 0.955 | 0.934 | 0.869 | 0.998 |
| IFIT1 IFI44L SIGLEC1 HERC6 SLC1A2 MMP8 | 0.857 | 0.848 | 0.866 | 0.895 | 0.835 | 0.955 | 0.908 | 0.836 | 0.979 |
| RSAD2 IFIT1 ISG15 SLC1A2 IL1R2 MMP8 | 0.857 | 0.848 | 0.865 | 0.903 | 0.845 | 0.960 | 0.954 | 0.909 | 1.000 |
| OAS1 IFIT1 IFI44L SLC1A2 IL1R2 MMP8 | 0.857 | 0.848 | 0.866 | 0.903 | 0.846 | 0.960 | 0.951 | 0.904 | 0.998 |
| RSAD2 OAS1 HERC6 SLC1A2 FAM20A RETN | 0.857 | 0.847 | 0.867 | 0.896 | 0.831 | 0.961 | 0.936 | 0.855 | 1 |
| RSAD2 HERC6 SLC1A2 IL1R2 RETN MMP8 | 0.857 | 0.847 | 0.866 | 0.901 | 0.842 | 0.959 | 0.936 | 0.869 | 1 |
| OAS1 IFI44L HERC6 SLC1A2 IL1R2 MMP8 | 0.857 | 0.847 | 0.866 | 0.900 | 0.844 | 0.956 | 0.934 | 0.872 | 0.995 |
| RSAD2 IFI27 OAS1 IFI44L SIGLEC1 SLC1A2 | 0.857 | 0.847 | 0.866 | 0.896 | 0.837 | 0.955 | 0.952 | 0.902 | 1 |
| IFI27 IFIT1 SIGLEC1 ISG15 HERC6 SLC1A2 | 0.857 | 0.848 | 0.866 | 0.896 | 0.838 | 0.954 | 0.952 | 0.904 | 1 |
| RSAD2 OAS1 IFIT1 ISG15 SLC1A2 IL1R2 | 0.857 | 0.848 | 0.866 | 0.899 | 0.840 | 0.958 | 0.948 | 0.898 | 0.998 |
| RSAD2 IFIT1 ISG15 SLC1A2 OLAH RETN | 0.857 | 0.847 | 0.866 | 0.906 | 0.847 | 0.965 | 0.947 | 0.898 | 0.996 |
| RSAD2 IFIT1 IFI44L SLC1A2 IL1R2 MMP8 | 0.857 | 0.848 | 0.865 | 0.902 | 0.845 | 0.960 | 0.954 | 0.909 | 1.000 |
| OAS1 IFIT1 HERC6 SLC1A2 FAM20A RETN | 0.857 | 0.846 | 0.867 | 0.897 | 0.832 | 0.961 | 0.939 | 0.858 | 1 |
| RSAD2 IFIT1 HERC6 OLAH RETN MMP8 | 0.857 | 0.846 | 0.867 | 0.890 | 0.821 | 0.960 | 0.955 | 0.913 | 0.998 |
| OAS1 IFI44L OLAH FAM20A RETN MMP8 | 0.857 | 0.846 | 0.867 | 0.896 | 0.825 | 0.966 | 0.955 | 0.904 | 1 |
| RSAD2 IFI44L SIGLEC1 HERC6 RETN MMP8 | 0.857 | 0.847 | 0.867 | 0.890 | 0.819 | 0.962 | 0.937 | 0.883 | 0.990 |
| RSAD2 IFI27 IFIT1 ISG15 SLC1A2 FAM20A | 0.857 | 0.846 | 0.867 | 0.889 | 0.820 | 0.958 | 0.939 | 0.860 | 1 |
| OAS1 IFIT1 SLC1A2 IL1R2 RETN MMP8 | 0.856 | 0.847 | 0.866 | 0.902 | 0.843 | 0.961 | 0.958 | 0.915 | 1 |
| RSAD2 IFI27 OAS1 IFIT1 SIGLEC1 SLC1A2 | 0.856 | 0.847 | 0.866 | 0.896 | 0.837 | 0.955 | 0.951 | 0.900 | 1 |
| RSAD2 OAS1 IFI44L HERC6 OLAH RETN | 0.856 | 0.846 | 0.866 | 0.891 | 0.822 | 0.960 | 0.940 | 0.888 | 0.992 |
| RSAD2 IFIT1 SLC1A2 OLAH RETN MMP8 | 0.856 | 0.847 | 0.866 | 0.900 | 0.839 | 0.961 | 0.953 | 0.909 | 0.998 |
| OAS1 IFI44L ISG15 OLAH FAM20A RETN | 0.856 | 0.846 | 0.867 | 0.890 | 0.817 | 0.963 | 0.954 | 0.901 | 1 |
| IFIT1 IFI44L HERC6 SLC1A2 FAM20A MMP8 | 0.856 | 0.846 | 0.866 | 0.895 | 0.834 | 0.955 | 0.917 | 0.830 | 1 |
| RSAD2 IFI27 OAS1 IFIT1 SIGLEC1 HERC6 | 0.856 | 0.847 | 0.866 | 0.896 | 0.842 | 0.951 | 0.916 | 0.851 | 0.982 |
| RSAD2 IFI44L HERC6 SLC1A2 IL1R2 MMP8 | 0.856 | 0.847 | 0.866 | 0.900 | 0.843 | 0.956 | 0.936 | 0.874 | 0.998 |
| RSAD2 IFI27 IFIT1 SIGLEC1 ISG15 HERC6 | 0.856 | 0.847 | 0.866 | 0.893 | 0.837 | 0.950 | 0.914 | 0.847 | 0.981 |
| IFI44L SIGLEC1 ISG15 HERC6 RETN MMP8 | 0.856 | 0.846 | 0.867 | 0.884 | 0.808 | 0.959 | 0.951 | 0.904 | 0.998 |
| IFIT1 IFI44L ISG15 HERC6 FAM20A MMP8 | 0.856 | 0.846 | 0.866 | 0.891 | 0.825 | 0.957 | 0.922 | 0.837 | 1 |
| IFI27 IFI44L SIGLEC1 ISG15 HERC6 SLC1A2 | 0.856 | 0.848 | 0.865 | 0.894 | 0.835 | 0.953 | 0.949 | 0.897 | 1 |
| RSAD2 IFI44L HERC6 FAM20A RETN MMP8 | 0.856 | 0.846 | 0.866 | 0.895 | 0.826 | 0.963 | 0.935 | 0.858 | 1 |
| RSAD2 IFIT1 ISG15 SLC1A2 IL1R2 RETN | 0.856 | 0.847 | 0.865 | 0.897 | 0.836 | 0.957 | 0.946 | 0.893 | 0.998 |
| RSAD2 OAS1 IFI44L SIGLEC1 ISG15 OLAH | 0.856 | 0.846 | 0.866 | 0.893 | 0.824 | 0.961 | 0.943 | 0.892 | 0.995 |
| RSAD2 IFIT1 IFI44L HERC6 OLAH RETN | 0.856 | 0.846 | 0.866 | 0.892 | 0.824 | 0.961 | 0.936 | 0.881 | 0.991 |
| RSAD2 IFI27 IFI44L SIGLEC1 ISG15 SLC1A2 | 0.856 | 0.847 | 0.865 | 0.896 | 0.837 | 0.955 | 0.953 | 0.906 | 1 |
| RSAD2 OAS1 ISG15 SLC1A2 OLAH RETN | 0.856 | 0.846 | 0.866 | 0.902 | 0.843 | 0.961 | 0.942 | 0.890 | 0.995 |
| IFI27 OAS1 IFIT1 IFI44L SIGLEC1 HERC6 | 0.856 | 0.846 | 0.865 | 0.897 | 0.842 | 0.953 | 0.932 | 0.874 | 0.989 |
| RSAD2 IFI27 OAS1 IFIT1 SLC1A2 FAM20A | 0.856 | 0.845 | 0.866 | 0.890 | 0.821 | 0.959 | 0.938 | 0.857 | 1 |
| OAS1 IFIT1 IFI44L SLC1A2 IL1R2 RETN | 0.856 | 0.846 | 0.865 | 0.899 | 0.839 | 0.959 | 0.945 | 0.893 | 0.996 |
| OAS1 IFIT1 IFI44L ISG15 SLC1A2 IL1R2 | 0.856 | 0.846 | 0.865 | 0.901 | 0.844 | 0.959 | 0.947 | 0.897 | 0.997 |
| RSAD2 OAS1 IFI44L HERC6 SLC1A2 FAM20A | 0.856 | 0.845 | 0.866 | 0.895 | 0.833 | 0.956 | 0.917 | 0.834 | 1 |
| RSAD2 OAS1 IFIT1 SLC1A2 IL1R2 RETN | 0.856 | 0.846 | 0.865 | 0.896 | 0.836 | 0.957 | 0.948 | 0.896 | 1.000 |
| RSAD2 IFI44L SIGLEC1 ISG15 OLAH RETN | 0.856 | 0.846 | 0.866 | 0.891 | 0.821 | 0.962 | 0.951 | 0.902 | 1 |
| OAS1 IFIT1 IL1R2 OLAH RETN MMP8 | 0.856 | 0.846 | 0.866 | 0.893 | 0.826 | 0.960 | 0.957 | 0.914 | 0.999 |
| OAS1 SIGLEC1 ISG15 HERC6 SLC1A2 MMP8 | 0.856 | 0.846 | 0.865 | 0.893 | 0.832 | 0.955 | 0.936 | 0.878 | 0.994 |
| IFI44L ISG15 SLC1A2 IL1R2 RETN MMP8 | 0.856 | 0.846 | 0.865 | 0.901 | 0.845 | 0.957 | 0.950 | 0.897 | 1 |
| RSAD2 OAS1 SLC1A2 OLAH RETN MMP8 | 0.856 | 0.846 | 0.865 | 0.901 | 0.842 | 0.961 | 0.947 | 0.897 | 0.996 |
| IFIT1 IFI44L SLC1A2 IL1R2 RETN MMP8 | 0.856 | 0.846 | 0.865 | 0.903 | 0.845 | 0.961 | 0.959 | 0.917 | 1.000 |
| RSAD2 IFIT1 IFI44L ISG15 SLC1A2 IL1R2 | 0.856 | 0.847 | 0.864 | 0.900 | 0.842 | 0.958 | 0.948 | 0.898 | 0.998 |
| IFIT1 ISG15 SLC1A2 IL1R2 RETN MMP8 | 0.856 | 0.846 | 0.865 | 0.904 | 0.846 | 0.961 | 0.959 | 0.918 | 1.000 |
| RSAD2 ISG15 SLC1A2 OLAH RETN MMP8 | 0.856 | 0.846 | 0.865 | 0.900 | 0.840 | 0.960 | 0.952 | 0.906 | 0.998 |
| IFI44L SIGLEC1 HERC6 SLC1A2 RETN MMP8 | 0.856 | 0.846 | 0.865 | 0.893 | 0.826 | 0.959 | 0.952 | 0.904 | 1.000 |
| RSAD2 IFI27 IFIT1 SIGLEC1 ISG15 SLC1A2 | 0.855 | 0.846 | 0.865 | 0.895 | 0.836 | 0.955 | 0.952 | 0.903 | 1 |
| RSAD2 IFI44L ISG15 HERC6 FAM20A MMP8 | 0.855 | 0.845 | 0.866 | 0.890 | 0.825 | 0.956 | 0.926 | 0.849 | 1 |
| RSAD2 IFI44L HERC6 OLAH RETN MMP8 | 0.855 | 0.845 | 0.866 | 0.892 | 0.823 | 0.962 | 0.949 | 0.902 | 0.996 |
| RSAD2 OAS1 IFIT1 IFI44L IL1R2 MMP8 | 0.855 | 0.846 | 0.865 | 0.893 | 0.828 | 0.957 | 0.954 | 0.911 | 0.998 |
| IFIT1 IFI44L OLAH FAM20A RETN MMP8 | 0.855 | 0.845 | 0.866 | 0.894 | 0.826 | 0.963 | 0.961 | 0.918 | 1 |
| RSAD2 IFI44L SIGLEC1 ISG15 OLAH MMP8 | 0.855 | 0.845 | 0.865 | 0.892 | 0.824 | 0.960 | 0.954 | 0.909 | 0.999 |
| RSAD2 OAS1 SIGLEC1 HERC6 SLC1A2 MMP8 | 0.855 | 0.846 | 0.865 | 0.889 | 0.825 | 0.952 | 0.936 | 0.879 | 0.993 |
| RSAD2 IFIT1 IFI44L ISG15 HERC6 FAM20A | 0.855 | 0.845 | 0.866 | 0.892 | 0.827 | 0.956 | 0.918 | 0.836 | 1 |
| OAS1 IFI44L HERC6 FAM20A RETN MMP8 | 0.855 | 0.845 | 0.866 | 0.896 | 0.826 | 0.965 | 0.943 | 0.868 | 1 |
| IFIT1 HERC6 SLC1A2 FAM20A RETN MMP8 | 0.855 | 0.845 | 0.866 | 0.895 | 0.829 | 0.960 | 0.940 | 0.862 | 1 |
| IFIT1 IFI44L ISG15 OLAH FAM20A RETN | 0.855 | 0.845 | 0.865 | 0.892 | 0.821 | 0.962 | 0.955 | 0.907 | 1 |
| OAS1 IFIT1 ISG15 IL1R2 OLAH MMP8 | 0.855 | 0.846 | 0.865 | 0.894 | 0.830 | 0.957 | 0.960 | 0.920 | 1.000 |
| SIGLEC1 ISG15 HERC6 SLC1A2 RETN MMP8 | 0.855 | 0.845 | 0.865 | 0.893 | 0.826 | 0.960 | 0.960 | 0.916 | 1 |
| RSAD2 IFIT1 SLC1A2 IL1R2 RETN MMP8 | 0.855 | 0.846 | 0.864 | 0.902 | 0.843 | 0.960 | 0.959 | 0.917 | 1 |
| RSAD2 OAS1 IFIT1 IFI44L SLC1A2 IL1R2 | 0.855 | 0.846 | 0.864 | 0.899 | 0.840 | 0.958 | 0.949 | 0.899 | 0.999 |
| RSAD2 OAS1 IFIT1 IL1R2 OLAH RETN | 0.855 | 0.845 | 0.865 | 0.891 | 0.822 | 0.960 | 0.950 | 0.904 | 0.996 |
| RSAD2 IFIT1 HERC6 SLC1A2 FAM20A RETN | 0.855 | 0.845 | 0.865 | 0.897 | 0.832 | 0.961 | 0.937 | 0.856 | 1 |
| RSAD2 IFI27 IFI44L SIGLEC1 ISG15 HERC6 | 0.855 | 0.846 | 0.864 | 0.895 | 0.838 | 0.951 | 0.912 | 0.844 | 0.980 |
| IFIT1 IFI44L ISG15 HERC6 OLAH MMP8 | 0.855 | 0.845 | 0.865 | 0.888 | 0.822 | 0.954 | 0.939 | 0.886 | 0.992 |
| OAS1 ISG15 SLC1A2 OLAH RETN MMP8 | 0.855 | 0.845 | 0.865 | 0.903 | 0.844 | 0.961 | 0.943 | 0.892 | 0.995 |
| RSAD2 SIGLEC1 ISG15 HERC6 SLC1A2 MMP8 | 0.855 | 0.846 | 0.864 | 0.891 | 0.828 | 0.954 | 0.928 | 0.868 | 0.988 |
| OAS1 IFIT1 IFI44L SIGLEC1 SLC1A2 RETN | 0.855 | 0.846 | 0.864 | 0.895 | 0.833 | 0.957 | 0.940 | 0.886 | 0.995 |
| IFIT1 IFI44L ISG15 IL1R2 OLAH MMP8 | 0.855 | 0.845 | 0.865 | 0.893 | 0.832 | 0.955 | 0.965 | 0.929 | 1 |
| RSAD2 IFIT1 IFI44L SLC1A2 IL1R2 RETN | 0.855 | 0.846 | 0.864 | 0.898 | 0.838 | 0.958 | 0.949 | 0.899 | 0.999 |
| OAS1 IFIT1 IFI44L IL1R2 OLAH MMP8 | 0.855 | 0.845 | 0.864 | 0.894 | 0.831 | 0.958 | 0.959 | 0.918 | 0.999 |
| IFI27 IFIT1 IFI44L SIGLEC1 ISG15 SLC1A2 | 0.855 | 0.846 | 0.864 | 0.895 | 0.835 | 0.955 | 0.954 | 0.905 | 1 |
| RSAD2 OAS1 IFIT1 ISG15 IL1R2 OLAH | 0.855 | 0.845 | 0.864 | 0.893 | 0.826 | 0.960 | 0.946 | 0.896 | 0.995 |
| OAS1 IFIT1 ISG15 HERC6 SLC1A2 RETN | 0.855 | 0.844 | 0.865 | 0.896 | 0.833 | 0.959 | 0.939 | 0.876 | 1 |
| OAS1 IFIT1 SIGLEC1 ISG15 SLC1A2 RETN | 0.855 | 0.845 | 0.864 | 0.896 | 0.834 | 0.958 | 0.948 | 0.898 | 0.998 |
| RSAD2 IFI44L HERC6 SLC1A2 FAM20A MMP8 | 0.855 | 0.844 | 0.865 | 0.895 | 0.834 | 0.956 | 0.915 | 0.830 | 1 |
| RSAD2 OAS1 IFIT1 IFI44L IL1R2 OLAH | 0.854 | 0.845 | 0.864 | 0.894 | 0.827 | 0.961 | 0.946 | 0.896 | 0.995 |
| RSAD2 OAS1 IFIT1 ISG15 HERC6 RETN | 0.854 | 0.844 | 0.865 | 0.890 | 0.822 | 0.958 | 0.939 | 0.883 | 0.995 |
| OAS1 HERC6 SLC1A2 FAM20A RETN MMP8 | 0.854 | 0.844 | 0.865 | 0.894 | 0.829 | 0.960 | 0.936 | 0.854 | 1 |
| IFI27 OAS1 IFI44L ISG15 SLC1A2 MMP8 | 0.854 | 0.845 | 0.864 | 0.894 | 0.832 | 0.956 | 0.914 | 0.841 | 0.988 |
| ISG15 SLC1A2 IL1R2 OLAH RETN MMP8 | 0.854 | 0.845 | 0.864 | 0.900 | 0.843 | 0.958 | 0.930 | 0.862 | 0.999 |
| RSAD2 IFI27 OAS1 IFI44L SLC1A2 MMP8 | 0.854 | 0.844 | 0.864 | 0.893 | 0.831 | 0.956 | 0.914 | 0.838 | 0.991 |
| RSAD2 OAS1 HERC6 FAM20A RETN MMP8 | 0.854 | 0.844 | 0.865 | 0.888 | 0.818 | 0.959 | 0.937 | 0.859 | 1 |
| OAS1 IFIT1 ISG15 IL1R2 OLAH RETN | 0.854 | 0.844 | 0.864 | 0.892 | 0.824 | 0.960 | 0.951 | 0.905 | 0.997 |
| IFI27 IFIT1 IFI44L SIGLEC1 HERC6 SLC1A2 | 0.854 | 0.845 | 0.863 | 0.896 | 0.837 | 0.954 | 0.949 | 0.899 | 0.999 |
| OAS1 IFIT1 HERC6 FAM20A RETN MMP8 | 0.854 | 0.844 | 0.864 | 0.889 | 0.818 | 0.960 | 0.938 | 0.860 | 1 |
| OAS1 IFIT1 IFI44L HERC6 OLAH MMP8 | 0.854 | 0.844 | 0.864 | 0.887 | 0.821 | 0.953 | 0.937 | 0.882 | 0.992 |
| RSAD2 OAS1 IFIT1 SIGLEC1 SLC1A2 RETN | 0.854 | 0.844 | 0.864 | 0.896 | 0.835 | 0.958 | 0.947 | 0.896 | 0.997 |
| RSAD2 OAS1 IL1R2 OLAH RETN MMP8 | 0.854 | 0.844 | 0.864 | 0.892 | 0.824 | 0.959 | 0.954 | 0.910 | 0.998 |
| RSAD2 OAS1 IFIT1 IFI44L IL1R2 RETN | 0.854 | 0.844 | 0.864 | 0.887 | 0.817 | 0.958 | 0.950 | 0.904 | 0.996 |
| RSAD2 OAS1 ISG15 HERC6 SLC1A2 RETN | 0.854 | 0.844 | 0.864 | 0.895 | 0.831 | 0.958 | 0.937 | 0.872 | 1 |
| RSAD2 IFI44L ISG15 HERC6 SLC1A2 RETN | 0.854 | 0.844 | 0.864 | 0.898 | 0.835 | 0.961 | 0.929 | 0.867 | 0.991 |
| RSAD2 HERC6 SLC1A2 FAM20A RETN MMP8 | 0.854 | 0.844 | 0.864 | 0.896 | 0.831 | 0.960 | 0.937 | 0.855 | 1 |
| RSAD2 ISG15 HERC6 SLC1A2 FAM20A MMP8 | 0.854 | 0.843 | 0.864 | 0.895 | 0.833 | 0.957 | 0.924 | 0.839 | 1 |
| RSAD2 OAS1 IFIT1 IL1R2 RETN MMP8 | 0.854 | 0.844 | 0.864 | 0.890 | 0.820 | 0.959 | 0.960 | 0.920 | 1.000 |
| RSAD2 IFI44L ISG15 OLAH FAM20A MMP8 | 0.854 | 0.843 | 0.864 | 0.889 | 0.819 | 0.960 | 0.959 | 0.914 | 1 |
| RSAD2 IFIT1 IFI44L ISG15 HERC6 RETN | 0.854 | 0.844 | 0.864 | 0.888 | 0.819 | 0.957 | 0.928 | 0.868 | 0.988 |
| RSAD2 OAS1 ISG15 IL1R2 OLAH MMP8 | 0.854 | 0.844 | 0.863 | 0.894 | 0.831 | 0.957 | 0.943 | 0.891 | 0.996 |
| RSAD2 IFI27 IFIT1 IFI44L SIGLEC1 HERC6 | 0.854 | 0.844 | 0.863 | 0.896 | 0.840 | 0.951 | 0.913 | 0.844 | 0.982 |
| IFI44L ISG15 IL1R2 OLAH RETN MMP8 | 0.854 | 0.844 | 0.863 | 0.893 | 0.829 | 0.956 | 0.964 | 0.926 | 1 |
| RSAD2 IFI44L ISG15 SLC1A2 IL1R2 RETN | 0.854 | 0.844 | 0.863 | 0.901 | 0.844 | 0.958 | 0.946 | 0.892 | 0.999 |
| RSAD2 OAS1 ISG15 HERC6 OLAH MMP8 | 0.853 | 0.844 | 0.863 | 0.889 | 0.824 | 0.954 | 0.945 | 0.895 | 0.994 |
| RSAD2 OAS1 IFIT1 ISG15 IL1R2 MMP8 | 0.853 | 0.844 | 0.863 | 0.892 | 0.828 | 0.956 | 0.958 | 0.916 | 0.999 |
| RSAD2 IFIT1 IFI44L OLAH FAM20A MMP8 | 0.853 | 0.843 | 0.864 | 0.888 | 0.817 | 0.959 | 0.957 | 0.909 | 1 |
| OAS1 IFIT1 IFI44L ISG15 IL1R2 MMP8 | 0.853 | 0.844 | 0.863 | 0.892 | 0.827 | 0.956 | 0.957 | 0.915 | 0.998 |
| RSAD2 IFIT1 IFI44L HERC6 SLC1A2 FAM20A | 0.853 | 0.843 | 0.864 | 0.898 | 0.840 | 0.957 | 0.918 | 0.834 | 1 |
| OAS1 IFI44L SIGLEC1 HERC6 SLC1A2 MMP8 | 0.853 | 0.844 | 0.863 | 0.888 | 0.824 | 0.951 | 0.935 | 0.877 | 0.993 |
| RSAD2 OAS1 IFIT1 HERC6 OLAH MMP8 | 0.853 | 0.843 | 0.863 | 0.887 | 0.820 | 0.954 | 0.936 | 0.881 | 0.991 |
| IFIT1 ISG15 IL1R2 OLAH RETN MMP8 | 0.853 | 0.843 | 0.864 | 0.892 | 0.827 | 0.958 | 0.966 | 0.931 | 1 |
| OAS1 IFIT1 IFI44L IL1R2 RETN MMP8 | 0.853 | 0.843 | 0.863 | 0.890 | 0.821 | 0.959 | 0.961 | 0.922 | 1.000 |
| IFI27 OAS1 IFIT1 ISG15 SLC1A2 MMP8 | 0.853 | 0.844 | 0.863 | 0.889 | 0.825 | 0.952 | 0.920 | 0.846 | 0.993 |
| OAS1 ISG15 HERC6 SLC1A2 RETN MMP8 | 0.853 | 0.843 | 0.863 | 0.894 | 0.829 | 0.958 | 0.937 | 0.872 | 1 |
| RSAD2 IFI27 IFIT1 IFI44L SIGLEC1 SLC1A2 | 0.853 | 0.844 | 0.862 | 0.896 | 0.837 | 0.955 | 0.952 | 0.904 | 1 |
| OAS1 IFIT1 SIGLEC1 SLC1A2 RETN MMP8 | 0.853 | 0.844 | 0.863 | 0.896 | 0.834 | 0.958 | 0.951 | 0.904 | 0.998 |
| IFIT1 IFI44L IL1R2 OLAH RETN MMP8 | 0.853 | 0.843 | 0.863 | 0.893 | 0.827 | 0.960 | 0.964 | 0.927 | 1 |
| RSAD2 OAS1 IFIT1 IFI44L ISG15 IL1R2 | 0.853 | 0.844 | 0.863 | 0.891 | 0.825 | 0.957 | 0.949 | 0.902 | 0.996 |
| OAS1 IFI44L SIGLEC1 ISG15 SLC1A2 RETN | 0.853 | 0.844 | 0.863 | 0.896 | 0.834 | 0.958 | 0.950 | 0.899 | 1 |
| IFI44L ISG15 HERC6 SLC1A2 FAM20A MMP8 | 0.853 | 0.843 | 0.864 | 0.889 | 0.824 | 0.955 | 0.928 | 0.844 | 1 |
| OAS1 IFIT1 ISG15 HERC6 OLAH MMP8 | 0.853 | 0.843 | 0.863 | 0.888 | 0.822 | 0.953 | 0.948 | 0.900 | 0.995 |
| IFIT1 IFI44L SIGLEC1 ISG15 SLC1A2 RETN | 0.853 | 0.844 | 0.862 | 0.897 | 0.835 | 0.958 | 0.942 | 0.890 | 0.995 |
| RSAD2 OAS1 IFI44L HERC6 OLAH MMP8 | 0.853 | 0.843 | 0.863 | 0.889 | 0.822 | 0.955 | 0.933 | 0.875 | 0.990 |
| OAS1 IFIT1 IFI44L IL1R2 OLAH RETN | 0.853 | 0.843 | 0.863 | 0.892 | 0.823 | 0.960 | 0.947 | 0.897 | 0.996 |
| RSAD2 OAS1 IFI44L IL1R2 OLAH MMP8 | 0.853 | 0.843 | 0.863 | 0.895 | 0.832 | 0.958 | 0.946 | 0.894 | 0.997 |
| OAS1 ISG15 HERC6 SLC1A2 FAM20A MMP8 | 0.853 | 0.843 | 0.863 | 0.894 | 0.832 | 0.956 | 0.925 | 0.840 | 1 |
| RSAD2 ISG15 OLAH FAM20A RETN MMP8 | 0.853 | 0.843 | 0.863 | 0.889 | 0.818 | 0.960 | 0.964 | 0.917 | 1 |
| RSAD2 IFIT1 ISG15 IL1R2 OLAH MMP8 | 0.853 | 0.843 | 0.863 | 0.891 | 0.828 | 0.955 | 0.963 | 0.925 | 1 |
| RSAD2 IFIT1 IFI44L SIGLEC1 SLC1A2 RETN | 0.853 | 0.844 | 0.862 | 0.894 | 0.832 | 0.956 | 0.940 | 0.886 | 0.994 |
| OAS1 IFIT1 IFI44L ISG15 IL1R2 OLAH | 0.853 | 0.843 | 0.862 | 0.892 | 0.825 | 0.960 | 0.947 | 0.898 | 0.996 |
| RSAD2 OAS1 ISG15 IL1R2 OLAH RETN | 0.853 | 0.843 | 0.863 | 0.892 | 0.824 | 0.960 | 0.947 | 0.897 | 0.997 |
| RSAD2 OAS1 IFIT1 IFI44L HERC6 OLAH | 0.853 | 0.843 | 0.863 | 0.886 | 0.820 | 0.953 | 0.934 | 0.876 | 0.992 |
| RSAD2 OAS1 IFIT1 ISG15 IL1R2 RETN | 0.853 | 0.843 | 0.863 | 0.890 | 0.821 | 0.959 | 0.955 | 0.913 | 0.998 |
| OAS1 IFIT1 ISG15 IL1R2 RETN MMP8 | 0.853 | 0.843 | 0.863 | 0.891 | 0.823 | 0.959 | 0.967 | 0.932 | 1 |
| RSAD2 IFI44L SIGLEC1 HERC6 SLC1A2 MMP8 | 0.853 | 0.843 | 0.862 | 0.890 | 0.826 | 0.953 | 0.927 | 0.867 | 0.988 |
| OAS1 IFI44L ISG15 SLC1A2 IL1R2 RETN | 0.853 | 0.843 | 0.862 | 0.902 | 0.846 | 0.959 | 0.942 | 0.889 | 0.996 |
| IFI44L ISG15 SLC1A2 FAM20A RETN MMP8 | 0.853 | 0.843 | 0.863 | 0.893 | 0.828 | 0.959 | 0.935 | 0.859 | 1 |
| IFIT1 ISG15 OLAH FAM20A RETN MMP8 | 0.853 | 0.842 | 0.863 | 0.888 | 0.818 | 0.959 | 0.965 | 0.924 | 1 |
| RSAD2 OAS1 ISG15 HERC6 FAM20A MMP8 | 0.853 | 0.842 | 0.863 | 0.886 | 0.819 | 0.953 | 0.926 | 0.844 | 1 |
| RSAD2 OAS1 IFI44L IL1R2 OLAH RETN | 0.853 | 0.843 | 0.863 | 0.892 | 0.824 | 0.960 | 0.945 | 0.893 | 0.996 |
| IFI27 IFIT1 IFI44L ISG15 SLC1A2 MMP8 | 0.853 | 0.843 | 0.863 | 0.892 | 0.830 | 0.954 | 0.920 | 0.849 | 0.991 |
| RSAD2 OAS1 ISG15 HERC6 SLC1A2 FAM20A | 0.853 | 0.842 | 0.863 | 0.887 | 0.824 | 0.950 | 0.928 | 0.844 | 1 |
| RSAD2 IFI27 IFI44L ISG15 SLC1A2 MMP8 | 0.853 | 0.843 | 0.863 | 0.893 | 0.831 | 0.955 | 0.916 | 0.843 | 0.989 |
| IFIT1 IFI44L SIGLEC1 ISG15 HERC6 MMP8 | 0.853 | 0.843 | 0.862 | 0.885 | 0.819 | 0.951 | 0.895 | 0.816 | 0.973 |
| IFI27 OAS1 IFIT1 IFI44L SLC1A2 MMP8 | 0.853 | 0.843 | 0.863 | 0.892 | 0.829 | 0.954 | 0.916 | 0.842 | 0.991 |
| OAS1 IFI44L ISG15 HERC6 RETN MMP8 | 0.853 | 0.842 | 0.863 | 0.893 | 0.824 | 0.961 | 0.936 | 0.877 | 0.994 |
| OAS1 IFI44L ISG15 HERC6 SLC1A2 FAM20A | 0.853 | 0.842 | 0.863 | 0.890 | 0.827 | 0.953 | 0.935 | 0.854 | 1 |
| OAS1 IFI44L IL1R2 OLAH RETN MMP8 | 0.852 | 0.842 | 0.863 | 0.892 | 0.825 | 0.959 | 0.955 | 0.912 | 0.999 |
| RSAD2 OAS1 SIGLEC1 ISG15 SLC1A2 RETN | 0.852 | 0.843 | 0.862 | 0.894 | 0.831 | 0.957 | 0.953 | 0.904 | 1 |
| RSAD2 IFIT1 IFI44L ISG15 OLAH FAM20A | 0.852 | 0.842 | 0.863 | 0.889 | 0.818 | 0.960 | 0.957 | 0.908 | 1 |
| RSAD2 OAS1 IFI44L ISG15 IL1R2 OLAH | 0.852 | 0.843 | 0.862 | 0.894 | 0.828 | 0.959 | 0.942 | 0.890 | 0.995 |
| OAS1 ISG15 IL1R2 OLAH RETN MMP8 | 0.852 | 0.842 | 0.862 | 0.894 | 0.828 | 0.960 | 0.957 | 0.914 | 0.999 |
| RSAD2 IFIT1 IFI44L IL1R2 OLAH MMP8 | 0.852 | 0.842 | 0.862 | 0.889 | 0.825 | 0.954 | 0.963 | 0.925 | 1 |
| IFIT1 IFI44L ISG15 IL1R2 RETN MMP8 | 0.852 | 0.842 | 0.862 | 0.892 | 0.825 | 0.959 | 0.967 | 0.933 | 1 |
| IFIT1 ISG15 HERC6 SLC1A2 FAM20A MMP8 | 0.852 | 0.842 | 0.862 | 0.892 | 0.828 | 0.955 | 0.922 | 0.836 | 1 |
| OAS1 IFIT1 IFI44L ISG15 IL1R2 RETN | 0.852 | 0.842 | 0.862 | 0.890 | 0.821 | 0.959 | 0.955 | 0.913 | 0.998 |
| RSAD2 IFI27 OAS1 IFIT1 SLC1A2 MMP8 | 0.852 | 0.842 | 0.862 | 0.890 | 0.827 | 0.954 | 0.918 | 0.843 | 0.994 |
| RSAD2 IFIT1 HERC6 FAM20A RETN MMP8 | 0.852 | 0.842 | 0.863 | 0.889 | 0.818 | 0.960 | 0.939 | 0.861 | 1 |
| RSAD2 OAS1 IFIT1 ISG15 HERC6 OLAH | 0.852 | 0.842 | 0.862 | 0.885 | 0.820 | 0.951 | 0.945 | 0.895 | 0.994 |
| RSAD2 OAS1 ISG15 HERC6 RETN MMP8 | 0.852 | 0.842 | 0.862 | 0.889 | 0.821 | 0.957 | 0.940 | 0.885 | 0.995 |
| RSAD2 IFI27 OAS1 ISG15 SLC1A2 MMP8 | 0.852 | 0.842 | 0.862 | 0.890 | 0.827 | 0.954 | 0.914 | 0.836 | 0.992 |
| OAS1 IFIT1 IFI44L ISG15 HERC6 OLAH | 0.852 | 0.842 | 0.862 | 0.886 | 0.821 | 0.951 | 0.950 | 0.904 | 0.996 |
| RSAD2 OAS1 ISG15 SLC1A2 IL1R2 RETN | 0.852 | 0.842 | 0.862 | 0.896 | 0.836 | 0.955 | 0.945 | 0.886 | 1 |
| RSAD2 IFI44L ISG15 OLAH FAM20A RETN | 0.852 | 0.841 | 0.863 | 0.890 | 0.820 | 0.961 | 0.958 | 0.912 | 1 |
| OAS1 SIGLEC1 ISG15 SLC1A2 RETN MMP8 | 0.852 | 0.842 | 0.862 | 0.894 | 0.832 | 0.957 | 0.957 | 0.911 | 1 |
| IFI27 IFIT1 IFI44L SIGLEC1 ISG15 HERC6 | 0.852 | 0.842 | 0.861 | 0.893 | 0.834 | 0.952 | 0.921 | 0.857 | 0.984 |
| RSAD2 IFIT1 ISG15 OLAH FAM20A MMP8 | 0.852 | 0.841 | 0.862 | 0.884 | 0.811 | 0.956 | 0.961 | 0.914 | 1 |
| OAS1 IFIT1 IFI44L SIGLEC1 HERC6 SLC1A2 | 0.852 | 0.843 | 0.861 | 0.890 | 0.830 | 0.950 | 0.927 | 0.868 | 0.986 |
| IFIT1 IFI44L SIGLEC1 SLC1A2 RETN MMP8 | 0.852 | 0.842 | 0.861 | 0.895 | 0.833 | 0.957 | 0.941 | 0.888 | 0.994 |
| OAS1 IFI44L HERC6 SLC1A2 FAM20A MMP8 | 0.852 | 0.841 | 0.862 | 0.892 | 0.829 | 0.954 | 0.927 | 0.845 | 1 |
| RSAD2 IFI44L ISG15 HERC6 RETN MMP8 | 0.852 | 0.841 | 0.862 | 0.892 | 0.824 | 0.960 | 0.933 | 0.875 | 0.991 |
| OAS1 IFIT1 ISG15 HERC6 RETN MMP8 | 0.852 | 0.841 | 0.862 | 0.889 | 0.821 | 0.957 | 0.938 | 0.882 | 0.995 |
| RSAD2 IFIT1 IFI44L ISG15 IL1R2 MMP8 | 0.852 | 0.842 | 0.861 | 0.891 | 0.827 | 0.955 | 0.961 | 0.922 | 1 |
| RSAD2 IFIT1 IFI44L HERC6 OLAH MMP8 | 0.852 | 0.842 | 0.861 | 0.887 | 0.821 | 0.953 | 0.935 | 0.879 | 0.991 |
| IFI44L SIGLEC1 ISG15 HERC6 SLC1A2 MMP8 | 0.851 | 0.842 | 0.861 | 0.887 | 0.822 | 0.952 | 0.937 | 0.880 | 0.994 |
| RSAD2 IFIT1 SIGLEC1 ISG15 SLC1A2 RETN | 0.851 | 0.842 | 0.861 | 0.896 | 0.834 | 0.958 | 0.949 | 0.900 | 0.998 |
| OAS1 IFI44L ISG15 HERC6 OLAH MMP8 | 0.851 | 0.841 | 0.862 | 0.888 | 0.823 | 0.954 | 0.948 | 0.900 | 0.996 |
| RSAD2 OAS1 IFI44L ISG15 SLC1A2 IL1R2 | 0.851 | 0.842 | 0.861 | 0.897 | 0.840 | 0.954 | 0.943 | 0.888 | 0.999 |
| RSAD2 OAS1 IFI44L ISG15 HERC6 OLAH | 0.851 | 0.841 | 0.861 | 0.889 | 0.825 | 0.953 | 0.941 | 0.890 | 0.993 |
| RSAD2 IFIT1 IL1R2 OLAH RETN MMP8 | 0.851 | 0.841 | 0.862 | 0.892 | 0.825 | 0.959 | 0.962 | 0.923 | 1 |
| RSAD2 IFIT1 ISG15 HERC6 OLAH MMP8 | 0.851 | 0.841 | 0.862 | 0.889 | 0.824 | 0.953 | 0.945 | 0.895 | 0.994 |
| OAS1 IFIT1 SIGLEC1 ISG15 HERC6 MMP8 | 0.851 | 0.842 | 0.861 | 0.886 | 0.823 | 0.949 | 0.896 | 0.817 | 0.974 |
| RSAD2 IFI44L ISG15 SLC1A2 IL1R2 MMP8 | 0.851 | 0.843 | 0.860 | 0.897 | 0.840 | 0.954 | 0.942 | 0.885 | 0.999 |
| RSAD2 OAS1 IFIT1 IFI44L HERC6 FAM20A | 0.851 | 0.841 | 0.862 | 0.884 | 0.816 | 0.953 | 0.923 | 0.841 | 1 |
| RSAD2 ISG15 IL1R2 OLAH RETN MMP8 | 0.851 | 0.841 | 0.861 | 0.892 | 0.826 | 0.958 | 0.968 | 0.933 | 1 |
| OAS1 IFI44L ISG15 IL1R2 OLAH MMP8 | 0.851 | 0.842 | 0.861 | 0.895 | 0.832 | 0.957 | 0.947 | 0.897 | 0.997 |
| RSAD2 OAS1 IFI44L SLC1A2 IL1R2 RETN | 0.851 | 0.842 | 0.861 | 0.896 | 0.837 | 0.955 | 0.941 | 0.884 | 0.999 |
| RSAD2 IFIT1 ISG15 HERC6 FAM20A MMP8 | 0.851 | 0.841 | 0.862 | 0.887 | 0.822 | 0.953 | 0.923 | 0.840 | 1 |
| RSAD2 OAS1 IFI44L SIGLEC1 SLC1A2 RETN | 0.851 | 0.842 | 0.861 | 0.893 | 0.829 | 0.956 | 0.948 | 0.897 | 0.999 |
| RSAD2 OAS1 IFIT1 SIGLEC1 HERC6 SLC1A2 | 0.851 | 0.842 | 0.860 | 0.889 | 0.829 | 0.950 | 0.929 | 0.872 | 0.987 |
| OAS1 IFIT1 SIGLEC1 ISG15 HERC6 SLC1A2 | 0.851 | 0.842 | 0.860 | 0.890 | 0.830 | 0.950 | 0.933 | 0.876 | 0.989 |
| IFIT1 IFI44L ISG15 HERC6 SLC1A2 FAM20A | 0.851 | 0.841 | 0.861 | 0.892 | 0.830 | 0.953 | 0.925 | 0.837 | 1 |
| RSAD2 ISG15 SLC1A2 IL1R2 RETN MMP8 | 0.85 | 0.842 | 0.860 | 0.897 | 0.838 | 0.956 | 0.946 | 0.889 | 1 |
| OAS1 IFIT1 ISG15 HERC6 FAM20A MMP8 | 0.851 | 0.840 | 0.862 | 0.887 | 0.821 | 0.953 | 0.926 | 0.844 | 1 |
| OAS1 IFI44L ISG15 IL1R2 OLAH RETN | 0.851 | 0.841 | 0.861 | 0.892 | 0.825 | 0.960 | 0.949 | 0.900 | 0.998 |
| RSAD2 IFI44L ISG15 HERC6 OLAH MMP8 | 0.851 | 0.841 | 0.861 | 0.890 | 0.826 | 0.954 | 0.938 | 0.885 | 0.991 |
| RSAD2 IFIT1 ISG15 OLAH FAM20A RETN | 0.851 | 0.841 | 0.861 | 0.888 | 0.814 | 0.961 | 0.959 | 0.909 | 1 |
| RSAD2 IFI44L OLAH FAM20A RETN MMP8 | 0.851 | 0.840 | 0.862 | 0.895 | 0.826 | 0.963 | 0.959 | 0.913 | 1 |
| RSAD2 IFI44L SIGLEC1 ISG15 SLC1A2 RETN | 0.851 | 0.841 | 0.860 | 0.896 | 0.833 | 0.958 | 0.948 | 0.898 | 0.997 |
| OAS1 IFIT1 IFI44L SIGLEC1 HERC6 MMP8 | 0.851 | 0.841 | 0.860 | 0.883 | 0.817 | 0.949 | 0.888 | 0.807 | 0.969 |
| RSAD2 IFIT1 ISG15 IL1R2 RETN MMP8 | 0.851 | 0.841 | 0.861 | 0.891 | 0.824 | 0.959 | 0.967 | 0.933 | 1 |
| OAS1 IFI44L SIGLEC1 SLC1A2 RETN MMP8 | 0.851 | 0.841 | 0.860 | 0.893 | 0.829 | 0.956 | 0.951 | 0.901 | 1 |
| OAS1 IFI44L ISG15 SLC1A2 IL1R2 MMP8 | 0.851 | 0.842 | 0.860 | 0.898 | 0.841 | 0.954 | 0.940 | 0.883 | 0.997 |
| IFIT1 IFI44L ISG15 IL1R2 OLAH RETN | 0.851 | 0.841 | 0.861 | 0.892 | 0.826 | 0.958 | 0.963 | 0.924 | 1 |
| RSAD2 IFIT1 IFI44L ISG15 IL1R2 OLAH | 0.851 | 0.841 | 0.860 | 0.892 | 0.827 | 0.958 | 0.953 | 0.909 | 0.998 |
| RSAD2 IFIT1 IFI44L ISG15 IL1R2 RETN | 0.851 | 0.841 | 0.860 | 0.886 | 0.817 | 0.955 | 0.957 | 0.914 | 0.999 |
| RSAD2 IFI27 IFIT1 IFI44L SLC1A2 MMP8 | 0.851 | 0.841 | 0.860 | 0.891 | 0.829 | 0.954 | 0.917 | 0.846 | 0.989 |
| RSAD2 IFIT1 ISG15 IL1R2 OLAH RETN | 0.850 | 0.841 | 0.860 | 0.890 | 0.823 | 0.958 | 0.962 | 0.923 | 1 |
| RSAD2 IFI27 IFIT1 ISG15 SLC1A2 MMP8 | 0.850 | 0.841 | 0.860 | 0.889 | 0.825 | 0.952 | 0.920 | 0.847 | 0.992 |
| RSAD2 IFIT1 IFI44L IL1R2 RETN MMP8 | 0.850 | 0.840 | 0.861 | 0.889 | 0.820 | 0.959 | 0.964 | 0.927 | 1 |
| RSAD2 OAS1 IFI44L ISG15 SLC1A2 FAM20A | 0.850 | 0.839 | 0.861 | 0.893 | 0.828 | 0.959 | 0.916 | 0.831 | 1 |
| RSAD2 OAS1 IFI44L HERC6 SLC1A2 RETN | 0.850 | 0.840 | 0.861 | 0.889 | 0.822 | 0.956 | 0.936 | 0.875 | 0.996 |
| RSAD2 IFIT1 OLAH FAM20A RETN MMP8 | 0.850 | 0.840 | 0.861 | 0.886 | 0.814 | 0.959 | 0.964 | 0.920 | 1 |
| OAS1 IFIT1 IFI44L HERC6 FAM20A MMP8 | 0.850 | 0.840 | 0.861 | 0.885 | 0.817 | 0.954 | 0.922 | 0.840 | 1 |
| RSAD2 IFI44L IL1R2 OLAH RETN MMP8 | 0.850 | 0.840 | 0.860 | 0.890 | 0.824 | 0.956 | 0.964 | 0.927 | 1 |
| IFIT1 SIGLEC1 ISG15 SLC1A2 RETN MMP8 | 0.850 | 0.841 | 0.860 | 0.895 | 0.833 | 0.957 | 0.951 | 0.904 | 0.998 |
| RSAD2 OAS1 IFIT1 SIGLEC1 SLC1A2 MMP8 | 0.850 | 0.841 | 0.860 | 0.887 | 0.824 | 0.950 | 0.934 | 0.877 | 0.991 |
| RSAD2 IFIT1 ISG15 HERC6 SLC1A2 RETN | 0.850 | 0.840 | 0.860 | 0.891 | 0.826 | 0.956 | 0.936 | 0.873 | 0.999 |
| RSAD2 IFIT1 IFI44L HERC6 FAM20A MMP8 | 0.850 | 0.840 | 0.861 | 0.886 | 0.818 | 0.954 | 0.916 | 0.833 | 0.999 |
| RSAD2 OAS1 IFIT1 ISG15 HERC6 FAM20A | 0.850 | 0.840 | 0.860 | 0.881 | 0.813 | 0.949 | 0.924 | 0.839 | 1 |
| RSAD2 IFIT1 IFI44L ISG15 HERC6 OLAH | 0.850 | 0.840 | 0.860 | 0.889 | 0.826 | 0.953 | 0.943 | 0.894 | 0.993 |
| RSAD2 IFIT1 IFI44L SIGLEC1 HERC6 SLC1A2 | 0.850 | 0.841 | 0.859 | 0.890 | 0.830 | 0.951 | 0.923 | 0.862 | 0.984 |

Performance obtained for the identification of the viral or bacterial nature of the infection on the basis of the seven-gene combinations:

TABLE 11

| | AUC | | | AUC | CI 95% | | AUC | CI 95% | |
|---|---|---|---|---|---|---|---|---|---|
| Model | model | CI 95% | | Train | Train | | Test | Test | |
| IFI27, SIGLEC1, ISG15, IL1R2, OLAH, FAM20A, | 0.904 | 0.895 | 0.912 | 0.932 | 0.879 | 0.985 | 0.948 | 0.882 | 1 |
| MMP8 | | | | | | | | | |
| IFI27, IFIT1, SIGLEC1, HERC6, OLAH, | 0.903 | 0.894 | 0.912 | 0.931 | 0.876 | 0.986 | 0.953 | 0.893 | 1 |
| FAM20A, MMP8 | | | | | | | | | |
| IFI27, IFIT1, SIGLEC1, IL1R2, OLAH, FAM20A, | 0.903 | 0.895 | 0.911 | 0.934 | 0.883 | 0.985 | 0.958 | 0.900 | 1 |
| MMP8 | | | | | | | | | |
| IFI27, OAS1, SIGLEC1, HERC6, IL1R2, | 0.903 | 0.894 | 0.912 | 0.933 | 0.882 | 0.984 | 0.951 | 0.887 | 1 |
| FAM20A, MMP8 | | | | | | | | | |
| IFI27, OAS1, IFIT1, SIGLEC1, HERC6, OLAH, | 0.903 | 0.894 | 0.912 | 0.931 | 0.878 | 0.984 | 0.939 | 0.875 | 1 |
| FAM20A | | | | | | | | | |
| IFI27, IFI44L, SIGLEC1, IL1R2, OLAH, | 0.903 | 0.895 | 0.911 | 0.934 | 0.884 | 0.984 | 0.954 | 0.895 | 1 |
| FAM20A, MMP8 | | | | | | | | | |
| IFI27, IFIT1, SIGLEC1, HERC6, IL1R2, | 0.903 | 0.895 | 0.911 | 0.933 | 0.883 | 0.983 | 0.953 | 0.887 | 1 |
| FAM20A, MMP8 | | | | | | | | | |
| RSAD2, IFI27, SIGLEC1, IL1R2, OLAH, | 0.903 | 0.894 | 0.911 | 0.935 | 0.884 | 0.986 | 0.952 | 0.892 | 1 |
| FAM20A, MMP8 | | | | | | | | | |
| IFI27, IFIT1, IFI44L, SIGLEC1, HERC6, OLAH, | 0.903 | 0.894 | 0.911 | 0.931 | 0.876 | 0.986 | 0.943 | 0.881 | 1 |
| FAM20A | | | | | | | | | |
| RSAD2, IFI27, SIGLEC1, HERC6, OLAH, | 0.902 | 0.893 | 0.911 | 0.934 | 0.880 | 0.987 | 0.950 | 0.891 | 1 |
| FAM20A, MMP8 | | | | | | | | | |
| IFI27, OAS1, SIGLEC1, IL1R2, OLAH, FAM20A, | 0.902 | 0.894 | 0.911 | 0.934 | 0.883 | 0.986 | 0.951 | 0.892 | 1 |
| MMP8 | | | | | | | | | |
| RSAD2, IFI27, SIGLEC1, HERC6, IL1R2, | 0.902 | 0.893 | 0.910 | 0.934 | 0.883 | 0.985 | 0.953 | 0.886 | 1 |
| FAM20A, MMP8 | | | | | | | | | |
| IFI27, IFIT1, SIGLEC1, HERC6, IL1R2, OLAH, | 0.902 | 0.893 | 0.910 | 0.932 | 0.879 | 0.985 | 0.943 | 0.879 | 1 |
| FAM20A | | | | | | | | | |
| IFI27, IFIT1, SIGLEC1, ISG15, HERC6, OLAH, | 0.901 | 0.893 | 0.910 | 0.932 | 0.877 | 0.986 | 0.941 | 0.878 | 1 |
| FAM20A | | | | | | | | | |
| IFI27, IFIT1, IFI44L, SIGLEC1, IL1R2, FAM20A, | 0.901 | 0.893 | 0.909 | 0.931 | 0.880 | 0.983 | 0.955 | 0.893 | 1 |
| MMP8 | | | | | | | | | |
| IFI27, IFI44L, SIGLEC1, ISG15, IL1R2, | 0.901 | 0.894 | 0.909 | 0.933 | 0.882 | 0.983 | 0.950 | 0.882 | 1 |
| FAM20A, MMP8 | | | | | | | | | |
| IFI27, IFI44L, SIGLEC1, HERC6, IL1R2, | 0.901 | 0.893 | 0.910 | 0.931 | 0.879 | 0.983 | 0.952 | 0.885 | 1 |
| FAM20A, MMP8 | | | | | | | | | |
| IFI27, IFIT1, SIGLEC1, ISG15, OLAH, FAM20A, | 0.901 | 0.892 | 0.910 | 0.929 | 0.871 | 0.986 | 0.951 | 0.894 | 1 |
| MMP8 | | | | | | | | | |
| IFI27, OAS1, SIGLEC1, HERC6, OLAH, | 0.901 | 0.892 | 0.910 | 0.928 | 0.871 | 0.985 | 0.948 | 0.888 | 1 |
| FAM20A, MMP8 | | | | | | | | | |
| IFI27, SIGLEC1, HERC6, IL1R2, OLAH, | 0.901 | 0.892 | 0.910 | 0.930 | 0.876 | 0.984 | 0.955 | 0.897 | 1 |
| FAM20A, MMP8 | | | | | | | | | |
| RSAD2, IFI27, IFIT1, SIGLEC1, HERC6, OLAH, | 0.901 | 0.892 | 0.909 | 0.932 | 0.879 | 0.985 | 0.940 | 0.877 | 1 |
| FAM20A | | | | | | | | | |
| RSAD2, IFI27, HERC6, IL1R2, OLAH, FAM20A, | 0.901 | 0.892 | 0.909 | 0.933 | 0.883 | 0.983 | 0.927 | 0.850 | 1 |
| MMP8 | | | | | | | | | |
| IFI27, IFI44L, SIGLEC1, ISG15, OLAH, | 0.901 | 0.892 | 0.909 | 0.927 | 0.870 | 0.985 | 0.948 | 0.888 | 1 |
| FAM20A, MMP8 | | | | | | | | | |
| IFI27, OAS1, IFIT1, SIGLEC1, IL1R2, FAM20A, | 0.901 | 0.892 | 0.909 | 0.932 | 0.880 | 0.984 | 0.955 | 0.894 | 1 |
| MMP8 | | | | | | | | | |
| IFI27, IFIT1, IFI44L, SIGLEC1, HERC6, IL1R2, | 0.900 | 0.892 | 0.908 | 0.932 | 0.881 | 0.983 | 0.948 | 0.880 | 1 |
| FAM20A | | | | | | | | | |
| IFI27, IFIT1, SIGLEC1, HERC6, OLAH, | 0.900 | 0.892 | 0.909 | 0.931 | 0.875 | 0.986 | 0.943 | 0.881 | 1 |
| FAM20A, RETN | | | | | | | | | |
| IFI27, OAS1, HERC6, IL1R2, OLAH, FAM20A, | 0.900 | 0.892 | 0.909 | 0.933 | 0.883 | 0.983 | 0.928 | 0.853 | 1 |
| MMP8 | | | | | | | | | |
| IFI27, OAS1, IFIT1, SIGLEC1, OLAH, FAM20A, | 0.900 | 0.891 | 0.909 | 0.926 | 0.868 | 0.985 | 0.954 | 0.898 | 1 |
| MMP8 | | | | | | | | | |
| IFI27, OAS1, SIGLEC1, HERC6, IL1R2, OLAH, | 0.900 | 0.892 | 0.909 | 0.929 | 0.875 | 0.983 | 0.941 | 0.875 | 1 |
| FAM20A | | | | | | | | | |
| IFI27, OAS1, IFIT1, SIGLEC1, HERC6, IL1R2, | 0.900 | 0.892 | 0.908 | 0.933 | 0.883 | 0.984 | 0.946 | 0.879 | 1 |
| FAM20A | | | | | | | | | |
| IFI27, IFIT1, SIGLEC1, ISG15, IL1R2, FAM20A, | 0.900 | 0.892 | 0.908 | 0.932 | 0.880 | 0.983 | 0.954 | 0.888 | 1 |
| MMP8 | | | | | | | | | |
| IFI27, SIGLEC1, ISG15, HERC6, IL1R2, | 0.900 | 0.891 | 0.908 | 0.930 | 0.877 | 0.984 | 0.953 | 0.885 | 1 |
| FAM20A, MMP8 | | | | | | | | | |
| RSAD2, IFI27, IFIT1, SIGLEC1, IL1R2, | 0.900 | 0.892 | 0.908 | 0.931 | 0.880 | 0.983 | 0.958 | 0.897 | 1 |
| FAM20A, MMP8 | | | | | | | | | |
| IFI27, IFIT1, IFI44L, SIGLEC1, HERC6, | 0.900 | 0.891 | 0.908 | 0.928 | 0.873 | 0.984 | 0.940 | 0.867 | 1 |
| FAM20A, RETN | | | | | | | | | |
| IFI27, IFIT1, SIGLEC1, ISG15, HERC6, IL1R2, | 0.900 | 0.892 | 0.908 | 0.933 | 0.881 | 0.984 | 0.948 | 0.880 | 1 |
| FAM20A | | | | | | | | | |
| RSAD2, IFI27, OAS1, SIGLEC1, HERC6, OLAH, | 0.900 | 0.891 | 0.908 | 0.930 | 0.875 | 0.984 | 0.938 | 0.872 | 1 |
| FAM20A | | | | | | | | | |
| IFI27, IFIT1, SIGLEC1, IL1R2, FAM20A, RETN, | 0.900 | 0.891 | 0.908 | 0.934 | 0.882 | 0.986 | 0.957 | 0.893 | 1 |
| MMP8 | | | | | | | | | |
| IFI27, IFIT1, IFI44L, SIGLEC1, OLAH, FAM20A, | 0.900 | 0.891 | 0.908 | 0.927 | 0.870 | 0.985 | 0.955 | 0.901 | 1 |
| MMP8 | | | | | | | | | |
| IFI27, OAS1, SIGLEC1, IL1R2, FAM20A, RETN, | 0.899 | 0.890 | 0.908 | 0.931 | 0.877 | 0.985 | 0.953 | 0.889 | 1 |
| MMP8 | | | | | | | | | |
| IFI27, IFI44L, SIGLEC1, HERC6, IL1R2, OLAH, | 0.899 | 0.891 | 0.908 | 0.927 | 0.873 | 0.982 | 0.947 | 0.881 | 1 |
| FAM20A | | | | | | | | | |
| RSAD2, IFI27, SIGLEC1, IL1R2, FAM20A, | 0.899 | 0.890 | 0.908 | 0.935 | 0.883 | 0.987 | 0.950 | 0.883 | 1 |
| RETN, MMP8 | | | | | | | | | |
| IFI27, OAS1, IFI44L, SIGLEC1, IL1R2, FAM20A, | 0.899 | 0.891 | 0.908 | 0.933 | 0.882 | 0.984 | 0.953 | 0.889 | 1 |
| MMP8 | | | | | | | | | |
| IFI27, ISG15, HERC6, IL1R2, OLAH, FAM20A, | 0.899 | 0.891 | 0.908 | 0.930 | 0.879 | 0.982 | 0.927 | 0.852 | 1 |
| MMP8 | | | | | | | | | |
| IFI27, IFI44L, SIGLEC1, IL1R2, FAM20A, | 0.899 | 0.891 | 0.908 | 0.934 | 0.883 | 0.986 | 0.953 | 0.885 | 1 |
| RETN, MMP8 | | | | | | | | | |
| RSAD2, IFI27, OAS1, SIGLEC1, IL1R2, | 0.899 | 0.891 | 0.908 | 0.933 | 0.880 | 0.985 | 0.954 | 0.891 | 1 |
| FAM20A, MMP8 | | | | | | | | | |
| IFI27, SIGLEC1, ISG15, HERC6, OLAH, | 0.899 | 0.889 | 0.909 | 0.924 | 0.864 | 0.984 | 0.951 | 0.892 | 1 |
| FAM20A, MMP8 | | | | | | | | | |
| IFI27, IFIT1, SIGLEC1, HERC6, IL1R2, | 0.899 | 0.891 | 0.907 | 0.932 | 0.879 | 0.985 | 0.948 | 0.880 | 1 |
| FAM20A, RETN | | | | | | | | | |
| RSAD2, IFI27, SIGLEC1, HERC6, IL1R2, OLAH, | 0.899 | 0.890 | 0.908 | 0.930 | 0.876 | 0.985 | 0.947 | 0.884 | 1 |
| FAM20A | | | | | | | | | |
| IFI27, IFI44L, SIGLEC1, ISG15, HERC6, OLAH, | 0.899 | 0.890 | 0.908 | 0.929 | 0.872 | 0.985 | 0.943 | 0.878 | 1 |
| FAM20A | | | | | | | | | |
| RSAD2, IFI27, SIGLEC1, ISG15, OLAH, | 0.899 | 0.890 | 0.908 | 0.931 | 0.874 | 0.988 | 0.943 | 0.880 | 1 |
| FAM20A, MMP8 | | | | | | | | | |
| IFI27, IFI44L, ISG15, IL1R2, OLAH, FAM20A, | 0.899 | 0.890 | 0.907 | 0.930 | 0.876 | 0.983 | 0.928 | 0.856 | 1 |
| MMP8 | | | | | | | | | |
| IFI27, OAS1, IFI44L, SIGLEC1, HERC6, IL1R2, | 0.899 | 0.891 | 0.907 | 0.933 | 0.884 | 0.983 | 0.946 | 0.879 | 1 |
| FAM20A | | | | | | | | | |
| IFI27, OAS1, SIGLEC1, ISG15, OLAH, FAM20A, | 0.899 | 0.890 | 0.908 | 0.925 | 0.865 | 0.985 | 0.948 | 0.888 | 1 |
| MMP8 | | | | | | | | | |
| IFI27, OAS1, SIGLEC1, ISG15, HERC6, OLAH, | 0.899 | 0.890 | 0.908 | 0.928 | 0.872 | 0.984 | 0.939 | 0.873 | 1 |
| FAM20A | | | | | | | | | |
| IFI27, IFIT1, IFI44L, SIGLEC1, ISG15, OLAH, | 0.899 | 0.890 | 0.907 | 0.926 | 0.868 | 0.984 | 0.943 | 0.884 | 1 |
| FAM20A | | | | | | | | | |
| IFI27, OAS1, IFI44L, SIGLEC1, HERC6, OLAH, | 0.899 | 0.890 | 0.907 | 0.930 | 0.876 | 0.984 | 0.940 | 0.874 | 1 |
| FAM20A | | | | | | | | | |
| IFI27, IFI44L, SIGLEC1, ISG15, IL1R2, OLAH, | 0.899 | 0.890 | 0.907 | 0.928 | 0.873 | 0.983 | 0.946 | 0.882 | 1 |
| FAM20A | | | | | | | | | |
| RSAD2, IFI27, IFI44L, SIGLEC1, IL1R2, | 0.899 | 0.890 | 0.907 | 0.931 | 0.880 | 0.983 | 0.952 | 0.886 | 1 |
| FAM20A, MMP8 | | | | | | | | | |
| IFI27, IFI44L, SIGLEC1, ISG15, HERC6, IL1R2, | 0.899 | 0.890 | 0.907 | 0.929 | 0.875 | 0.982 | 0.941 | 0.870 | 1 |
| FAM20A | | | | | | | | | |
| IFI27, OAS1, IFIT1, SIGLEC1, HERC6, FAM20A, | 0.898 | 0.890 | 0.907 | 0.930 | 0.877 | 0.984 | 0.936 | 0.864 | 1 |
| RETN | | | | | | | | | |
| IFI27, IFI44L, HERC6, IL1R2, OLAH, FAM20A, | 0.898 | 0.890 | 0.907 | 0.931 | 0.880 | 0.983 | 0.936 | 0.864 | 1 |
| MMP8 | | | | | | | | | |
| RSAD2, IFI27, IFIT1, SIGLEC1, OLAH, | 0.898 | 0.890 | 0.907 | 0.928 | 0.870 | 0.985 | 0.954 | 0.899 | 1 |
| FAM20A, MMP8 | | | | | | | | | |
| RSAD2, IFI27, IFIT1, SIGLEC1, HERC6, IL1R2, | 0.898 | 0.890 | 0.907 | 0.932 | 0.881 | 0.983 | 0.949 | 0.883 | 1 |
| FAM20A | | | | | | | | | |
| IFI27, IFIT1, SIGLEC1, HERC6, FAM20A, | 0.898 | 0.889 | 0.907 | 0.926 | 0.869 | 0.984 | 0.949 | 0.878 | 1 |
| RETN, MMP8 | | | | | | | | | |
| RSAD2, IFI27, OAS1, SIGLEC1, HERC6, IL1R2, | 0.898 | 0.890 | 0.907 | 0.930 | 0.878 | 0.983 | 0.948 | 0.882 | 1 |
| FAM20A | | | | | | | | | |
| RSAD2, IFI27, SIGLEC1, ISG15, IL1R2, | 0.898 | 0.890 | 0.907 | 0.934 | 0.883 | 0.985 | 0.952 | 0.885 | 1 |
| FAM20A, MMP8 | | | | | | | | | |
| IFI27, IFIT1, HERC6, IL1R2, OLAH, FAM20A, | 0.898 | 0.890 | 0.907 | 0.932 | 0.880 | 0.983 | 0.937 | 0.867 | 1 |
| MMP8 | | | | | | | | | |
| IFI27, IFIT1, SIGLEC1, ISG15, IL1R2, OLAH, | 0.898 | 0.890 | 0.906 | 0.928 | 0.872 | 0.985 | 0.943 | 0.879 | 1 |
| FAM20A | | | | | | | | | |
| IFI27, SIGLEC1, ISG15, IL1R2, FAM20A, RETN, | 0.898 | 0.889 | 0.907 | 0.933 | 0.879 | 0.986 | 0.954 | 0.886 | 1 |
| MMP8 | | | | | | | | | |
| IFI27, OAS1, SIGLEC1, HERC6, OLAH, | 0.898 | 0.889 | 0.907 | 0.930 | 0.875 | 0.984 | 0.939 | 0.874 | 1 |
| FAM20A, RETN | | | | | | | | | |
| RSAD2, IFI27, OAS1, SIGLEC1, OLAH, | 0.898 | 0.889 | 0.907 | 0.927 | 0.869 | 0.985 | 0.951 | 0.895 | 1 |
| FAM20A, MMP8 | | | | | | | | | |
| RSAD2, IFI27, IFI44L, SIGLEC1, HERC6, IL1R2, | 0.898 | 0.890 | 0.906 | 0.930 | 0.877 | 0.982 | 0.945 | 0.876 | 1 |
| FAM20A | | | | | | | | | |
| IFI27, OAS1, IFIT1, IFI44L, SIGLEC1, OLAH, | 0.898 | 0.890 | 0.907 | 0.923 | 0.863 | 0.983 | 0.951 | 0.892 | 1 |
| FAM20A | | | | | | | | | |
| RSAD2, IFI27, OAS1, HERC6, OLAH, FAM20A, | 0.898 | 0.889 | 0.907 | 0.929 | 0.876 | 0.982 | 0.932 | 0.861 | 1 |
| MMP8 | | | | | | | | | |
| IFI27, IFIT1, IFI44L, SIGLEC1, IL1R2, OLAH, | 0.898 | 0.890 | 0.906 | 0.927 | 0.870 | 0.983 | 0.947 | 0.884 | 1 |
| FAM20A | | | | | | | | | |
| IFI27, OAS1, IFIT1, SIGLEC1, IL1R2, OLAH, | 0.898 | 0.890 | 0.906 | 0.926 | 0.869 | 0.983 | 0.949 | 0.887 | 1 |
| FAM20A | | | | | | | | | |
| IFI27, IFI44L, SIGLEC1, HERC6, OLAH, | 0.898 | 0.889 | 0.907 | 0.925 | 0.866 | 0.984 | 0.952 | 0.891 | 1 |
| FAM20A, MMP8 | | | | | | | | | |
| IFI27, SIGLEC1, IL1R2, OLAH, FAM20A, RETN, | 0.898 | 0.889 | 0.907 | 0.932 | 0.877 | 0.986 | 0.954 | 0.893 | 1 |
| MMP8 | | | | | | | | | |
| IFI27, OAS1, SIGLEC1, ISG15, IL1R2, FAM20A, | 0.898 | 0.889 | 0.907 | 0.931 | 0.880 | 0.983 | 0.951 | 0.885 | 1 |
| MMP8 | | | | | | | | | |
| IFI27, SIGLEC1, ISG15, SLC1A2, OLAH, | 0.898 | 0.888 | 0.907 | 0.935 | 0.886 | 0.985 | 0.941 | 0.874 | 1 |
| FAM20A, MMP8 | | | | | | | | | |
| RSAD2, IFI27, OAS1, HERC6, IL1R2, OLAH, | 0.898 | 0.889 | 0.906 | 0.929 | 0.877 | 0.981 | 0.927 | 0.853 | 1 |
| FAM20A | | | | | | | | | |
| IFI27, IFIT1, SIGLEC1, ISG15, HERC6, | 0.898 | 0.889 | 0.906 | 0.928 | 0.872 | 0.983 | 0.941 | 0.869 | 1 |
| FAM20A, RETN | | | | | | | | | |
| IFI27, IFIT1, SIGLEC1, OLAH, FAM20A, RETN, | 0.897 | 0.888 | 0.907 | 0.929 | 0.870 | 0.987 | 0.960 | 0.907 | 1 |
| MMP8 | | | | | | | | | |
| RSAD2, IFI27, SIGLEC1, ISG15, HERC6, OLAH, | 0.897 | 0.888 | 0.907 | 0.930 | 0.875 | 0.985 | 0.940 | 0.875 | 1 |
| FAM20A | | | | | | | | | |
| RSAD2, IFI27, IFI44L, SIGLEC1, HERC6, | 0.897 | 0.889 | 0.906 | 0.931 | 0.876 | 0.985 | 0.943 | 0.880 | 1 |
| OLAH, FAM20A | | | | | | | | | |
| IFI27, OAS1, IFIT1, HERC6, OLAH, FAM20A, | 0.897 | 0.888 | 0.907 | 0.927 | 0.872 | 0.982 | 0.935 | 0.867 | 1 |
| MMP8 | | | | | | | | | |
| IFI27, OAS1, IFIT1, IFI44L, HERC6, OLAH, | 0.897 | 0.888 | 0.906 | 0.928 | 0.873 | 0.983 | 0.932 | 0.860 | 1 |
| FAM20A | | | | | | | | | |
| IFI27, SIGLEC1, ISG15, OLAH, FAM20A, RETN, | 0.897 | 0.887 | 0.907 | 0.927 | 0.868 | 0.985 | 0.957 | 0.899 | 1 |
| MMP8 | | | | | | | | | |
| IFI27, OAS1, SIGLEC1, HERC6, IL1R2, | 0.897 | 0.888 | 0.906 | 0.932 | 0.881 | 0.983 | 0.945 | 0.877 | 1 |
| FAM20A, RETN | | | | | | | | | |
| RSAD2, IFI27, IFI44L, HERC6, OLAH, FAM20A, | 0.897 | 0.888 | 0.907 | 0.929 | 0.875 | 0.984 | 0.934 | 0.866 | 1 |
| MMP8 | | | | | | | | | |
| IFI27, SIGLEC1, ISG15, HERC6, IL1R2, OLAH, | 0.897 | 0.888 | 0.906 | 0.928 | 0.873 | 0.983 | 0.948 | 0.886 | 1 |
| FAM20A | | | | | | | | | |
| IFI27, OAS1, IFIT1, SIGLEC1, ISG15, OLAH, | 0.897 | 0.888 | 0.906 | 0.926 | 0.868 | 0.984 | 0.943 | 0.882 | 1 |
| FAM20A | | | | | | | | | |
| IFI27, IFIT1, IFI44L, HERC6, IL1R2, OLAH, | 0.897 | 0.888 | 0.906 | 0.929 | 0.875 | 0.982 | 0.928 | 0.853 | 1 |
| FAM20A | | | | | | | | | |
| RSAD2, IFI27, SIGLEC1, HERC6, OLAH, | 0.897 | 0.888 | 0.906 | 0.929 | 0.874 | 0.984 | 0.938 | 0.872 | 1 |
| FAM20A, RETN | | | | | | | | | |
| IFI27, OAS1, SIGLEC1, ISG15, HERC6, IL1R2, | 0.897 | 0.888 | 0.906 | 0.930 | 0.877 | 0.982 | 0.949 | 0.883 | 1 |
| FAM20A | | | | | | | | | |
| IFI27, OAS1, SIGLEC1, HERC6, FAM20A, | 0.897 | 0.887 | 0.907 | 0.929 | 0.872 | 0.986 | 0.943 | 0.872 | 1 |
| RETN, MMP8 | | | | | | | | | |
| IFI27, OAS1, IFIT1, HERC6, IL1R2, OLAH, | 0.897 | 0.888 | 0.906 | 0.930 | 0.878 | 0.981 | 0.928 | 0.854 | 1 |
| FAM20A | | | | | | | | | |
| IFI27, IFIT1, SIGLEC1, ISG15, OLAH, FAM20A, | 0.897 | 0.888 | 0.906 | 0.926 | 0.868 | 0.985 | 0.943 | 0.882 | 1 |
| RETN | | | | | | | | | |
| IFI27, IFIT1, IFI44L, SIGLEC1, OLAH, FAM20A, | 0.897 | 0.888 | 0.905 | 0.924 | 0.864 | 0.984 | 0.950 | 0.891 | 1 |
| RETN | | | | | | | | | |
| IFI27, OAS1, SIGLEC1, OLAH, FAM20A, RETN, | 0.897 | 0.887 | 0.906 | 0.928 | 0.870 | 0.986 | 0.952 | 0.894 | 1 |
| MMP8 | | | | | | | | | |
| RSAD2, IFI27, SIGLEC1, HERC6, IL1R2, | 0.897 | 0.888 | 0.906 | 0.932 | 0.880 | 0.984 | 0.946 | 0.879 | 1 |
| FAM20A, RETN | | | | | | | | | |
| IFI27, IFI44L, ISG15, HERC6, IL1R2, OLAH, | 0.897 | 0.888 | 0.905 | 0.929 | 0.875 | 0.982 | 0.928 | 0.855 | 1 |
| FAM20A | | | | | | | | | |
| IFI27, IFIT1, IFI44L, IL1R2, OLAH, FAM20A, | 0.897 | 0.888 | 0.905 | 0.929 | 0.876 | 0.983 | 0.936 | 0.868 | 1 |
| MMP8 | | | | | | | | | |
| RSAD2, IFI27, IFIT1, HERC6, OLAH, FAM20A, | 0.896 | 0.887 | 0.905 | 0.929 | 0.876 | 0.982 | 0.934 | 0.865 | 1 |
| MMP8 | | | | | | | | | |
| IFI27, OAS1, ISG15, HERC6, OLAH, FAM20A, | 0.896 | 0.887 | 0.906 | 0.926 | 0.872 | 0.981 | 0.932 | 0.862 | 1 |
| MMP8 | | | | | | | | | |
| IFI27, IFIT1, SIGLEC1, IL1R2, OLAH, FAM20A, | 0.896 | 0.888 | 0.905 | 0.927 | 0.871 | 0.984 | 0.948 | 0.886 | 1 |
| RETN | | | | | | | | | |
| IFI27, IFIT1, IFI44L, SIGLEC1, ISG15, IL1R2, | 0.896 | 0.888 | 0.904 | 0.928 | 0.873 | 0.983 | 0.949 | 0.886 | 1 |
| FAM20A | | | | | | | | | |
| IFI27, OAS1, ISG15, HERC6, IL1R2, OLAH, | 0.896 | 0.888 | 0.905 | 0.929 | 0.876 | 0.981 | 0.926 | 0.852 | 1 |
| FAM20A | | | | | | | | | |
| IFI27, OAS1, IFI44L, HERC6, IL1R2, OLAH, | 0.896 | 0.887 | 0.905 | 0.930 | 0.877 | 0.982 | 0.926 | 0.850 | 1 |
| FAM20A | | | | | | | | | |
| IFI27, SIGLEC1, HERC6, IL1R2, FAM20A, | 0.896 | 0.887 | 0.906 | 0.931 | 0.877 | 0.985 | 0.955 | 0.888 | 1 |
| RETN, MMP8 | | | | | | | | | |
| RSAD2, IFI27, SIGLEC1, ISG15, HERC6, IL1R2, | 0.896 | 0.887 | 0.905 | 0.930 | 0.876 | 0.983 | 0.947 | 0.880 | 1 |
| FAM20A | | | | | | | | | |
| IFI27, IFIT1, IFI44L, HERC6, OLAH, FAM20A, | 0.896 | 0.887 | 0.905 | 0.925 | 0.869 | 0.982 | 0.937 | 0.871 | 1 |
| MMP8 | | | | | | | | | |
| IFI27, OAS1, IFI44L, SIGLEC1, OLAH, | 0.896 | 0.887 | 0.905 | 0.924 | 0.864 | 0.984 | 0.953 | 0.898 | 1 |
| FAM20A, MMP8 | | | | | | | | | |
| RSAD2, IFI27, OAS1, IFI44L, HERC6, OLAH, | 0.896 | 0.887 | 0.905 | 0.929 | 0.875 | 0.983 | 0.930 | 0.859 | 1 |
| FAM20A | | | | | | | | | |
| RSAD2, IFI27, IFIT1, SIGLEC1, IL1R2, OLAH, | 0.896 | 0.888 | 0.904 | 0.928 | 0.871 | 0.984 | 0.948 | 0.886 | 1 |
| FAM20A | | | | | | | | | |
| RSAD2, IFI27, SIGLEC1, OLAH, FAM20A, | 0.896 | 0.887 | 0.905 | 0.928 | 0.870 | 0.986 | 0.958 | 0.905 | 1 |
| RETN, MMP8 | | | | | | | | | |
| IFI27, IFIT1, ISG15, HERC6, IL1R2, OLAH, | 0.896 | 0.887 | 0.905 | 0.929 | 0.876 | 0.983 | 0.927 | 0.853 | 1 |
| FAM20A | | | | | | | | | |
| IFI27, OAS1, IFI44L, HERC6, OLAH, FAM20A, | 0.896 | 0.886 | 0.906 | 0.927 | 0.872 | 0.983 | 0.935 | 0.867 | 1 |
| MMP8 | | | | | | | | | |
| IFI27, OAS1, SIGLEC1, ISG15, IL1R2, OLAH, | 0.896 | 0.887 | 0.905 | 0.926 | 0.869 | 0.983 | 0.946 | 0.882 | 1 |
| FAM20A | | | | | | | | | |
| IFI27, IFI44L, ISG15, HERC6, OLAH, FAM20A, | 0.896 | 0.886 | 0.906 | 0.925 | 0.868 | 0.982 | 0.938 | 0.873 | 1 |
| MMP8 | | | | | | | | | |
| IFI27, IFIT1, ISG15, IL1R2, OLAH, FAM20A, | 0.896 | 0.887 | 0.905 | 0.928 | 0.875 | 0.982 | 0.934 | 0.864 | 1 |
| MMP8 | | | | | | | | | |
| IFI27, ISG15, HERC6, OLAH, FAM20A, RETN, | 0.896 | 0.886 | 0.906 | 0.927 | 0.870 | 0.984 | 0.937 | 0.869 | 1 |
| MMP8 | | | | | | | | | |
| RSAD2, IFI27, ISG15, HERC6, IL1R2, OLAH, | 0.896 | 0.887 | 0.905 | 0.930 | 0.878 | 0.982 | 0.929 | 0.857 | 1 |
| FAM20A | | | | | | | | | |
| IFI27, IFIT1, SIGLEC1, SLC1A2, OLAH, | 0.896 | 0.887 | 0.905 | 0.933 | 0.881 | 0.985 | 0.951 | 0.889 | 1 |
| FAM20A, MMP8 | | | | | | | | | |
| RSAD2, IFI27, IFI44L, SIGLEC1, OLAH, | 0.896 | 0.887 | 0.905 | 0.927 | 0.869 | 0.985 | 0.954 | 0.900 | 1 |
| FAM20A, MMP8 | | | | | | | | | |
| RSAD2, IFI27, HERC6, OLAH, FAM20A, RETN, | 0.896 | 0.887 | 0.905 | 0.931 | 0.877 | 0.985 | 0.930 | 0.858 | 1 |
| MMP8 | | | | | | | | | |
| IFI27, IFI44L, SIGLEC1, HERC6, IL1R2, | 0.896 | 0.887 | 0.905 | 0.930 | 0.877 | 0.983 | 0.942 | 0.870 | 1 |
| FAM20A, RETN | | | | | | | | | |
| RSAD2, IFI27, ISG15, HERC6, OLAH, FAM20A, | 0.896 | 0.887 | 0.905 | 0.927 | 0.872 | 0.982 | 0.930 | 0.861 | 1.000 |
| MMP8 | | | | | | | | | |
| IFI27, IFIT1, IFI44L, ISG15, HERC6, OLAH, | 0.896 | 0.887 | 0.905 | 0.926 | 0.869 | 0.982 | 0.933 | 0.864 | 1 |
| FAM20A | | | | | | | | | |
| IFI27, OAS1, IFIT1, IL1R2, OLAH, FAM20A, | 0.896 | 0.887 | 0.905 | 0.927 | 0.873 | 0.982 | 0.935 | 0.867 | 1 |
| MMP8 | | | | | | | | | |
| IFI27, OAS1, SIGLEC1, SLC1A2, OLAH, | 0.896 | 0.887 | 0.905 | 0.935 | 0.884 | 0.986 | 0.942 | 0.877 | 1 |
| FAM20A, MMP8 | | | | | | | | | |
| IFI27, OAS1, IFIT1, IFI44L, SIGLEC1, IL1R2, | 0.896 | 0.888 | 0.904 | 0.927 | 0.872 | 0.983 | 0.949 | 0.886 | 1 |
| FAM20A | | | | | | | | | |
| RSAD2, IFI27, IFIT1, IFI44L, HERC6, OLAH, | 0.896 | 0.887 | 0.905 | 0.929 | 0.874 | 0.983 | 0.928 | 0.858 | 0.998 |
| FAM20A | | | | | | | | | |
| IFI27, OAS1, IFI44L, SIGLEC1, IL1R2, OLAH, | 0.896 | 0.887 | 0.904 | 0.926 | 0.869 | 0.982 | 0.952 | 0.892 | 1 |
| FAM20A | | | | | | | | | |
| RSAD2, IFI27, IFI44L, HERC6, IL1R2, OLAH, | 0.896 | 0.887 | 0.905 | 0.929 | 0.876 | 0.983 | 0.928 | 0.856 | 1 |
| FAM20A | | | | | | | | | |
| IFI27, OAS1, IFIT1, SIGLEC1, OLAH, FAM20A, | 0.896 | 0.887 | 0.905 | 0.924 | 0.865 | 0.983 | 0.951 | 0.891 | 1 |
| RETN | | | | | | | | | |
| RSAD2, IFI27, OAS1, IL1R2, OLAH, FAM20A, | 0.896 | 0.887 | 0.905 | 0.929 | 0.876 | 0.982 | 0.933 | 0.861 | 1 |
| MMP8 | | | | | | | | | |
| IFI27, IFIT1, IFI44L, SIGLEC1, IL1R2, FAM20A, | 0.896 | 0.887 | 0.904 | 0.927 | 0.872 | 0.982 | 0.949 | 0.886 | 1 |
| RETN | | | | | | | | | |
| IFI27, IFI44L, SIGLEC1, OLAH, FAM20A, | 0.896 | 0.886 | 0.905 | 0.928 | 0.871 | 0.985 | 0.953 | 0.894 | 1 |
| RETN, MMP8 | | | | | | | | | |
| RSAD2, IFI27, IFIT1, SIGLEC1, HERC6, | 0.896 | 0.887 | 0.904 | 0.930 | 0.876 | 0.983 | 0.935 | 0.861 | 1 |
| FAM20A, RETN | | | | | | | | | |
| IFI27, IFIT1, ISG15, HERC6, OLAH, FAM20A, | 0.896 | 0.886 | 0.905 | 0.926 | 0.870 | 0.982 | 0.935 | 0.868 | 1 |
| MMP8 | | | | | | | | | |
| RSAD2, IFI27, IFIT1, HERC6, IL1R2, OLAH, | 0.896 | 0.887 | 0.904 | 0.929 | 0.876 | 0.982 | 0.929 | 0.857 | 1 |
| FAM20A | | | | | | | | | |
| IFI27, OAS1, HERC6, OLAH, FAM20A, RETN, | 0.896 | 0.886 | 0.905 | 0.931 | 0.877 | 0.986 | 0.929 | 0.854 | 1 |
| MMP8 | | | | | | | | | |
| IFI27, OAS1, IFI44L, ISG15, HERC6, OLAH, | 0.896 | 0.886 | 0.905 | 0.928 | 0.873 | 0.983 | 0.930 | 0.859 | 1 |
| FAM20A | | | | | | | | | |
| RSAD2, IFI27, SIGLEC1, HERC6, FAM20A, | 0.895 | 0.886 | 0.905 | 0.932 | 0.877 | 0.986 | 0.946 | 0.872 | 1 |
| RETN, MMP8 | | | | | | | | | |
| RSAD2, IFI27, IFIT1, IL1R2, OLAH, FAM20A, | 0.895 | 0.887 | 0.904 | 0.928 | 0.875 | 0.982 | 0.935 | 0.867 | 1 |
| MMP8 | | | | | | | | | |
| IFI27, OAS1, IFI44L, SIGLEC1, HERC6, | 0.895 | 0.887 | 0.904 | 0.928 | 0.873 | 0.982 | 0.938 | 0.864 | 1 |
| FAM20A, RETN | | | | | | | | | |
| IFI27, OAS1, SIGLEC1, ISG15, HERC6, | 0.895 | 0.886 | 0.904 | 0.929 | 0.873 | 0.984 | 0.938 | 0.864 | 1 |
| FAM20A, RETN | | | | | | | | | |
| RSAD2, IFI27, IFIT1, IFI44L, SIGLEC1, OLAH, | 0.895 | 0.887 | 0.904 | 0.925 | 0.866 | 0.984 | 0.950 | 0.893 | 1 |
| FAM20A | | | | | | | | | |
| IFI27, IFI44L, SIGLEC1, ISG15, OLAH, | 0.895 | 0.886 | 0.904 | 0.925 | 0.868 | 0.983 | 0.946 | 0.884 | 1 |
| FAM20A, RETN | | | | | | | | | |
| IFI27, SIGLEC1, ISG15, IL1R2, OLAH, FAM20A, | 0.895 | 0.887 | 0.904 | 0.926 | 0.869 | 0.983 | 0.946 | 0.883 | 1 |
| RETN | | | | | | | | | |
| RSAD2, IFI27, SIGLEC1, SLC1A2, IL1R2, | 0.895 | 0.886 | 0.904 | 0.935 | 0.889 | 0.981 | 0.954 | 0.886 | 1 |
| FAM20A, MMP8 | | | | | | | | | |
| RSAD2, IFI27, OAS1, IFIT1, HERC6, OLAH, | 0.895 | 0.886 | 0.904 | 0.928 | 0.874 | 0.982 | 0.929 | 0.857 | 1 |
| FAM20A | | | | | | | | | |
| IFI27, OAS1, SIGLEC1, SLC1A2, IL1R2, | 0.895 | 0.886 | 0.904 | 0.935 | 0.889 | 0.982 | 0.950 | 0.884 | 1 |
| FAM20A, MMP8 | | | | | | | | | |
| IFI27, OAS1, IFIT1, HERC6, OLAH, FAM20A, | 0.895 | 0.886 | 0.904 | 0.931 | 0.879 | 0.983 | 0.933 | 0.862 | 1 |
| RETN | | | | | | | | | |
| IFI27, OAS1, IFI44L, SIGLEC1, ISG15, OLAH, | 0.895 | 0.886 | 0.904 | 0.924 | 0.865 | 0.983 | 0.947 | 0.886 | 1 |
| FAM20A | | | | | | | | | |
| IFI27, IFIT1, SIGLEC1, ISG15, SLC1A2, OLAH, | 0.895 | 0.886 | 0.904 | 0.935 | 0.885 | 0.985 | 0.938 | 0.871 | 1 |
| FAM20A | | | | | | | | | |
| IFI27, IFI44L, SIGLEC1, IL1R2, OLAH, | 0.895 | 0.887 | 0.904 | 0.926 | 0.870 | 0.982 | 0.951 | 0.889 | 1 |
| FAM20A, RETN | | | | | | | | | |
| RSAD2, IFI27, OAS1, IFIT1, SIGLEC1, OLAH, | 0.895 | 0.886 | 0.904 | 0.923 | 0.864 | 0.983 | 0.950 | 0.891 | 1 |
| FAM20A | | | | | | | | | |
| IFI27, OAS1, SIGLEC1, IL1R2, OLAH, FAM20A, | 0.895 | 0.886 | 0.904 | 0.924 | 0.866 | 0.982 | 0.948 | 0.886 | 1 |
| RETN | | | | | | | | | |
| RSAD2, IFI27, OAS1, ISG15, HERC6, OLAH, | 0.895 | 0.886 | 0.904 | 0.928 | 0.874 | 0.982 | 0.927 | 0.855 | 1.000 |
| FAM20A | | | | | | | | | |
| RSAD2, IFI27, IFIT1, SIGLEC1, ISG15, OLAH, | 0.895 | 0.886 | 0.904 | 0.926 | 0.868 | 0.985 | 0.941 | 0.879 | 1 |
| FAM20A | | | | | | | | | |
| IFI27, OAS1, ISG15, IL1R2, OLAH, FAM20A, | 0.895 | 0.886 | 0.904 | 0.929 | 0.875 | 0.982 | 0.933 | 0.864 | 1 |
| MMP8 | | | | | | | | | |
| IFI27, IFI44L, SIGLEC1, HERC6, OLAH, | 0.895 | 0.886 | 0.904 | 0.924 | 0.866 | 0.982 | 0.947 | 0.879 | 1 |
| FAM20A, RETN | | | | | | | | | |
| IFI27, IFIT1, HERC6, OLAH, FAM20A, RETN, | 0.895 | 0.885 | 0.904 | 0.928 | 0.871 | 0.984 | 0.938 | 0.870 | 1 |
| MMP8 | | | | | | | | | |
| RSAD2, IFI27, OAS1, SIGLEC1, IL1R2, OLAH, | 0.895 | 0.886 | 0.904 | 0.925 | 0.867 | 0.983 | 0.947 | 0.883 | 1 |
| FAM20A | | | | | | | | | |
| RSAD2, IFI27, HERC6, IL1R2, OLAH, FAM20A, | 0.895 | 0.886 | 0.904 | 0.929 | 0.877 | 0.982 | 0.928 | 0.855 | 1 |
| RETN | | | | | | | | | |
| IFI27, IFIT1, SIGLEC1, HERC6, SLC1A2, OLAH, | 0.895 | 0.885 | 0.904 | 0.934 | 0.884 | 0.984 | 0.945 | 0.882 | 1 |
| FAM20A | | | | | | | | | |
| IFI27, IFIT1, IFI44L, HERC6, OLAH, FAM20A, | 0.895 | 0.886 | 0.904 | 0.927 | 0.872 | 0.982 | 0.934 | 0.863 | 1 |
| RETN | | | | | | | | | |
| IFI27, HERC6, IL1R2, OLAH, FAM20A, RETN, | 0.895 | 0.886 | 0.904 | 0.932 | 0.879 | 0.985 | 0.934 | 0.861 | 1 |
| MMP8 | | | | | | | | | |
| RSAD2, IFI27, SIGLEC1, SLC1A2, OLAH, | 0.895 | 0.885 | 0.904 | 0.933 | 0.880 | 0.985 | 0.947 | 0.884 | 1 |
| FAM20A, MMP8 | | | | | | | | | |
| IFI27, IFI44L, SIGLEC1, ISG15, IL1R2, | 0.895 | 0.886 | 0.903 | 0.929 | 0.874 | 0.983 | 0.947 | 0.880 | 1 |
| FAM20A, RETN | | | | | | | | | |
| IFI27, OAS1, IFIT1, ISG15, HERC6, OLAH, | 0.895 | 0.886 | 0.904 | 0.928 | 0.874 | 0.983 | 0.928 | 0.856 | 1 |
| FAM20A | | | | | | | | | |
| IFI27, IFIT1, SIGLEC1, ISG15, IL1R2, FAM20A, | 0.895 | 0.886 | 0.903 | 0.927 | 0.873 | 0.982 | 0.948 | 0.883 | 1 |
| RETN | | | | | | | | | |
| IFI27, SIGLEC1, ISG15, HERC6, OLAH, | 0.895 | 0.885 | 0.904 | 0.922 | 0.863 | 0.982 | 0.946 | 0.884 | 1 |
| FAM20A, RETN | | | | | | | | | |
| IFI27, IFIT1, IFI44L, SIGLEC1, SLC1A2, OLAH, | 0.895 | 0.886 | 0.903 | 0.931 | 0.878 | 0.984 | 0.943 | 0.879 | 1 |
| FAM20A | | | | | | | | | |
| RSAD2, IFI27, IFI44L, IL1R2, OLAH, FAM20A, | 0.895 | 0.885 | 0.904 | 0.928 | 0.873 | 0.983 | 0.932 | 0.859 | 1 |
| MMP8 | | | | | | | | | |
| IFI27, IFI44L, IL1R2, OLAH, FAM20A, RETN, | 0.895 | 0.885 | 0.904 | 0.930 | 0.876 | 0.984 | 0.933 | 0.863 | 1 |
| MMP8 | | | | | | | | | |
| RSAD2, IFI27, SIGLEC1, ISG15, IL1R2, OLAH, | 0.894 | 0.886 | 0.903 | 0.926 | 0.869 | 0.983 | 0.943 | 0.879 | 1 |
| FAM20A | | | | | | | | | |
| RSAD2, IFI27, IFI44L, ISG15, HERC6, OLAH, | 0.894 | 0.885 | 0.904 | 0.930 | 0.876 | 0.984 | 0.928 | 0.858 | 0.999 |
| FAM20A | | | | | | | | | |
| IFI27, ISG15, HERC6, IL1R2, OLAH, FAM20A, | 0.894 | 0.886 | 0.903 | 0.928 | 0.874 | 0.982 | 0.932 | 0.860 | 1 |
| RETN | | | | | | | | | |
| IFI27, OAS1, ISG15, HERC6, OLAH, FAM20A, | 0.894 | 0.885 | 0.904 | 0.930 | 0.878 | 0.983 | 0.935 | 0.866 | 1 |
| RETN | | | | | | | | | |
| IFI27, OAS1, SIGLEC1, HERC6, SLC1A2, | 0.894 | 0.885 | 0.904 | 0.931 | 0.880 | 0.983 | 0.935 | 0.866 | 1 |
| OLAH, FAM20A | | | | | | | | | |
| IFI27, IFI44L, SIGLEC1, SLC1A2, OLAH, | 0.894 | 0.885 | 0.903 | 0.927 | 0.873 | 0.982 | 0.953 | 0.892 | 1 |
| FAM20A, MMP8 | | | | | | | | | |
| RSAD2, IFI27, IFI44L, SIGLEC1, IL1R2, OLAH, | 0.894 | 0.886 | 0.903 | 0.926 | 0.870 | 0.983 | 0.950 | 0.888 | 1 |
| FAM20A | | | | | | | | | |
| IFI27, OAS1, IFIT1, SIGLEC1, IL1R2, FAM20A, | 0.894 | 0.886 | 0.903 | 0.927 | 0.872 | 0.983 | 0.949 | 0.887 | 1 |
| RETN | | | | | | | | | |
| IFI27, IFIT1, ISG15, HERC6, OLAH, FAM20A, | 0.894 | 0.885 | 0.903 | 0.926 | 0.871 | 0.981 | 0.933 | 0.863 | 1 |
| RETN | | | | | | | | | |
| RSAD2, IFI27, OAS1, SIGLEC1, ISG15, OLAH, | 0.894 | 0.885 | 0.903 | 0.924 | 0.865 | 0.983 | 0.946 | 0.886 | 1 |
| FAM20A | | | | | | | | | |
| IFI27, OAS1, HERC6, IL1R2, OLAH, FAM20A, | 0.894 | 0.885 | 0.903 | 0.931 | 0.879 | 0.982 | 0.929 | 0.855 | 1 |
| RETN | | | | | | | | | |
| IFI27, OAS1, IFI44L, SIGLEC1, ISG15, IL1R2, | 0.894 | 0.886 | 0.903 | 0.929 | 0.876 | 0.982 | 0.948 | 0.883 | 1 |
| FAM20A | | | | | | | | | |
| IFI27, OAS1, IFIT1, SIGLEC1, ISG15, IL1R2, | 0.894 | 0.886 | 0.903 | 0.927 | 0.871 | 0.982 | 0.949 | 0.886 | 1 |
| FAM20A | | | | | | | | | |
| IFI27, OAS1, IFIT1, HERC6, IL1R2, FAM20A, | 0.894 | 0.885 | 0.903 | 0.930 | 0.879 | 0.980 | 0.929 | 0.848 | 1 |
| MMP8 | | | | | | | | | |
| RSAD2, IFI27, ISG15, IL1R2, OLAH, FAM20A, | 0.894 | 0.885 | 0.903 | 0.929 | 0.876 | 0.982 | 0.927 | 0.854 | 1.000 |
| MMP8 | | | | | | | | | |
| IFI27, IFI44L, SIGLEC1, SLC1A2, IL1R2, | 0.894 | 0.886 | 0.903 | 0.935 | 0.890 | 0.981 | 0.953 | 0.885 | 1 |
| FAM20A, MMP8 | | | | | | | | | |
| IFI27, OAS1, IFI44L, HERC6, OLAH, FAM20A, | 0.894 | 0.885 | 0.903 | 0.930 | 0.876 | 0.983 | 0.934 | 0.864 | 1 |
| RETN | | | | | | | | | |
| IFI27, SIGLEC1, ISG15, SLC1A2, IL1R2, | 0.894 | 0.885 | 0.903 | 0.937 | 0.894 | 0.980 | 0.955 | 0.885 | 1 |
| FAM20A, MMP8 | | | | | | | | | |
| IFI27, IFIT1, SIGLEC1, SLC1A2, IL1R2, | 0.894 | 0.885 | 0.903 | 0.936 | 0.891 | 0.981 | 0.954 | 0.886 | 1 |
| FAM20A, MMP8 | | | | | | | | | |
| RSAD2, IFI27, OAS1, SIGLEC1, HERC6, | 0.894 | 0.885 | 0.903 | 0.929 | 0.874 | 0.984 | 0.938 | 0.864 | 1 |
| FAM20A, RETN | | | | | | | | | |
| IFI27, SIGLEC1, SLC1A2, IL1R2, OLAH, | 0.894 | 0.885 | 0.903 | 0.933 | 0.885 | 0.982 | 0.947 | 0.878 | 1 |
| FAM20A, MMP8 | | | | | | | | | |
| IFI27, OAS1, IFI44L, IL1R2, OLAH, FAM20A, | 0.894 | 0.884 | 0.903 | 0.929 | 0.874 | 0.983 | 0.932 | 0.859 | 1 |
| MMP8 | | | | | | | | | |
| IFI27, ISG15, IL1R2, OLAH, FAM20A, RETN, | 0.894 | 0.885 | 0.903 | 0.929 | 0.875 | 0.982 | 0.925 | 0.849 | 1 |
| MMP8 | | | | | | | | | |
| RSAD2, IFI27, SIGLEC1, IL1R2, OLAH, | 0.894 | 0.885 | 0.903 | 0.927 | 0.869 | 0.984 | 0.948 | 0.885 | 1 |
| FAM20A, RETN | | | | | | | | | |
| IFI27, IFI44L, ISG15, HERC6, OLAH, FAM20A, | 0.894 | 0.884 | 0.903 | 0.925 | 0.869 | 0.981 | 0.935 | 0.866 | 1 |
| RETN | | | | | | | | | |
| IFI27, OAS1, IFI44L, HERC6, IL1R2, FAM20A, | 0.894 | 0.885 | 0.903 | 0.930 | 0.879 | 0.980 | 0.926 | 0.840 | 1 |
| MMP8 | | | | | | | | | |
| RSAD2, IFI27, OAS1, IFI44L, SIGLEC1, OLAH, | 0.894 | 0.885 | 0.903 | 0.924 | 0.865 | 0.983 | 0.950 | 0.891 | 1 |
| FAM20A | | | | | | | | | |
| IFI27, IFI44L, ISG15, SLC1A2, OLAH, FAM20A, | 0.894 | 0.884 | 0.903 | 0.929 | 0.876 | 0.981 | 0.923 | 0.846 | 1.000 |
| MMP8 | | | | | | | | | |
| IFI27, IFI44L, SLC1A2, IL1R2, OLAH, FAM20A, | 0.894 | 0.885 | 0.902 | 0.932 | 0.882 | 0.982 | 0.924 | 0.844 | 1 |
| MMP8 | | | | | | | | | |
| RSAD2, IFI27, IFIT1, IFI44L, SIGLEC1, IL1R2, | 0.894 | 0.885 | 0.902 | 0.927 | 0.872 | 0.982 | 0.950 | 0.887 | 1 |
| FAM20A | | | | | | | | | |
| RSAD2, IFI27, SIGLEC1, ISG15, HERC6, | 0.894 | 0.884 | 0.903 | 0.930 | 0.876 | 0.983 | 0.946 | 0.876 | 1 |
| FAM20A, RETN | | | | | | | | | |
| IFI27, OAS1, HERC6, IL1R2, FAM20A, RETN, | 0.894 | 0.885 | 0.902 | 0.932 | 0.880 | 0.983 | 0.929 | 0.847 | 1 |
| MMP8 | | | | | | | | | |
| IFI27, OAS1, SIGLEC1, ISG15, OLAH, FAM20A, | 0.894 | 0.884 | 0.903 | 0.923 | 0.863 | 0.983 | 0.947 | 0.886 | 1 |
| RETN | | | | | | | | | |
| IFI27, IFIT1, IFI44L, SIGLEC1, ISG15, FAM20A, | 0.893 | 0.885 | 0.902 | 0.920 | 0.861 | 0.980 | 0.945 | 0.876 | 1 |
| RETN | | | | | | | | | |
| RSAD2, IFI27, OAS1, HERC6, OLAH, FAM20A, | 0.893 | 0.884 | 0.903 | 0.931 | 0.879 | 0.983 | 0.934 | 0.864 | 1 |
| RETN | | | | | | | | | |
| IFI27, IFIT1, IFI44L, HERC6, IL1R2, FAM20A, | 0.893 | 0.885 | 0.902 | 0.930 | 0.879 | 0.980 | 0.934 | 0.853 | 1 |
| MMP8 | | | | | | | | | |
| RSAD2, IFI27, IFI44L, SIGLEC1, ISG15, OLAH, | 0.893 | 0.884 | 0.902 | 0.924 | 0.865 | 0.982 | 0.943 | 0.881 | 1 |
| FAM20A | | | | | | | | | |
| RSAD2, IFI27, IFI44L, HERC6, IL1R2, FAM20A, | 0.893 | 0.885 | 0.902 | 0.928 | 0.876 | 0.979 | 0.926 | 0.840 | 1 |
| MMP8 | | | | | | | | | |
| RSAD2, IFI27, IFIT1, SIGLEC1, OLAH, | 0.893 | 0.884 | 0.902 | 0.923 | 0.864 | 0.983 | 0.948 | 0.887 | 1 |
| FAM20A, RETN | | | | | | | | | |
| IFI27, IFI44L, SIGLEC1, ISG15, SLC1A2, OLAH, | 0.893 | 0.885 | 0.902 | 0.931 | 0.880 | 0.983 | 0.939 | 0.871 | 1 |
| FAM20A | | | | | | | | | |
| RSAD2, IFI27, IFIT1, HERC6, OLAH, FAM20A, | 0.893 | 0.884 | 0.902 | 0.929 | 0.876 | 0.982 | 0.935 | 0.867 | 1 |
| RETN | | | | | | | | | |
| RSAD2, IFI27, HERC6, IL1R2, FAM20A, RETN, | 0.893 | 0.884 | 0.902 | 0.932 | 0.881 | 0.983 | 0.932 | 0.851 | 1 |
| MMP8 | | | | | | | | | |
| RSAD2, IFI27, OAS1, IFI44L, SIGLEC1, IL1R2, | 0.893 | 0.885 | 0.902 | 0.927 | 0.873 | 0.982 | 0.949 | 0.885 | 1 |
| FAM20A | | | | | | | | | |
| RSAD2, IFI27, IFIT1, ISG15, HERC6, OLAH, | 0.893 | 0.884 | 0.903 | 0.928 | 0.873 | 0.982 | 0.929 | 0.859 | 1.000 |
| FAM20A | | | | | | | | | |
| IFI27, IFI44L, ISG15, HERC6, IL1R2, FAM20A, | 0.893 | 0.884 | 0.902 | 0.930 | 0.880 | 0.980 | 0.927 | 0.841 | 1 |
| MMP8 | | | | | | | | | |
| IFI27, SIGLEC1, HERC6, OLAH, FAM20A, | 0.893 | 0.883 | 0.904 | 0.925 | 0.867 | 0.984 | 0.953 | 0.895 | 1 |
| RETN, MMP8 | | | | | | | | | |
| IFI27, IFIT1, IFI44L, ISG15, OLAH, FAM20A, | 0.893 | 0.884 | 0.902 | 0.921 | 0.861 | 0.981 | 0.936 | 0.870 | 1 |
| MMP8 | | | | | | | | | |
| RSAD2, IFI27, ISG15, HERC6, OLAH, FAM20A, | 0.893 | 0.884 | 0.902 | 0.929 | 0.875 | 0.982 | 0.935 | 0.867 | 1 |
| RETN | | | | | | | | | |
| IFI27, OAS1, IFI44L, SIGLEC1, IL1R2, FAM20A, | 0.893 | 0.884 | 0.902 | 0.929 | 0.876 | 0.983 | 0.948 | 0.883 | 1 |
| RETN | | | | | | | | | |
| RSAD2, IFI27, IFI44L, HERC6, OLAH, FAM20A, | 0.893 | 0.884 | 0.902 | 0.930 | 0.877 | 0.983 | 0.932 | 0.862 | 1 |
| RETN | | | | | | | | | |
| IFI27, IFIT1, IL1R2, OLAH, FAM20A, RETN, | 0.893 | 0.884 | 0.902 | 0.929 | 0.875 | 0.983 | 0.933 | 0.863 | 1 |
| MMP8 | | | | | | | | | |
| RSAD2, IFI27, OAS1, IFIT1, SIGLEC1, IL1R2, | 0.893 | 0.884 | 0.901 | 0.927 | 0.872 | 0.982 | 0.951 | 0.890 | 1 |
| FAM20A | | | | | | | | | |
| RSAD2, IFI27, OAS1, SIGLEC1, ISG15, IL1R2, | 0.893 | 0.884 | 0.902 | 0.927 | 0.872 | 0.983 | 0.949 | 0.882 | 1 |
| FAM20A | | | | | | | | | |
| RSAD2, IFI27, SIGLEC1, ISG15, OLAH, | 0.893 | 0.884 | 0.902 | 0.924 | 0.865 | 0.983 | 0.945 | 0.883 | 1 |
| FAM20A, RETN | | | | | | | | | |
| IFI27, OAS1, SIGLEC1, ISG15, IL1R2, FAM20A, | 0.893 | 0.884 | 0.902 | 0.928 | 0.872 | 0.983 | 0.949 | 0.882 | 1 |
| RETN | | | | | | | | | |
| IFI27, OAS1, SIGLEC1, ISG15, SLC1A2, OLAH, | 0.893 | 0.883 | 0.902 | 0.933 | 0.882 | 0.985 | 0.937 | 0.869 | 1 |
| FAM20A | | | | | | | | | |
| IFI27, IFI44L, ISG15, OLAH, FAM20A, RETN, | 0.893 | 0.883 | 0.902 | 0.924 | 0.865 | 0.983 | 0.940 | 0.874 | 1 |
| MMP8 | | | | | | | | | |
| IFI27, IFIT1, SIGLEC1, SLC1A2, IL1R2, OLAH, | 0.893 | 0.884 | 0.901 | 0.933 | 0.882 | 0.983 | 0.943 | 0.879 | 1 |
| FAM20A | | | | | | | | | |
| IFI27, IFI44L, HERC6, OLAH, FAM20A, RETN, | 0.893 | 0.883 | 0.903 | 0.926 | 0.869 | 0.984 | 0.941 | 0.874 | 1 |
| MMP8 | | | | | | | | | |
| IFI27, IFIT1, HERC6, IL1R2, OLAH, FAM20A, | 0.893 | 0.884 | 0.902 | 0.929 | 0.875 | 0.982 | 0.929 | 0.855 | 1 |
| RETN | | | | | | | | | |
| IFI27, OAS1, IFIT1, IFI44L, HERC6, IL1R2, | 0.893 | 0.884 | 0.901 | 0.927 | 0.875 | 0.978 | 0.927 | 0.844 | 1 |
| FAM20A | | | | | | | | | |
| IFI27, OAS1, IFI44L, ISG15, HERC6, IL1R2, | 0.893 | 0.884 | 0.901 | 0.927 | 0.876 | 0.979 | 0.925 | 0.840 | 1 |
| FAM20A | | | | | | | | | |
| RSAD2, IFI27, IFIT1, HERC6, IL1R2, FAM20A, | 0.892 | 0.884 | 0.901 | 0.930 | 0.879 | 0.980 | 0.933 | 0.852 | 1 |
| MMP8 | | | | | | | | | |
| RSAD2, IFI27, OAS1, SIGLEC1, OLAH, | 0.892 | 0.883 | 0.902 | 0.923 | 0.863 | 0.984 | 0.950 | 0.891 | 1 |
| FAM20A, RETN | | | | | | | | | |
| IFI27, OAS1, IFIT1, SIGLEC1, HERC6, OLAH, | 0.892 | 0.884 | 0.901 | 0.925 | 0.872 | 0.978 | 0.947 | 0.893 | 1 |
| RETN | | | | | | | | | |
| IFI27, ISG15, HERC6, IL1R2, FAM20A, RETN, | 0.892 | 0.883 | 0.902 | 0.931 | 0.880 | 0.983 | 0.932 | 0.851 | 1 |
| MMP8 | | | | | | | | | |
| IFI27, OAS1, HERC6, SLC1A2, OLAH, FAM20A, | 0.892 | 0.883 | 0.902 | 0.930 | 0.879 | 0.980 | 0.921 | 0.839 | 1 |
| MMP8 | | | | | | | | | |
| RSAD2, IFI27, IFIT1, SIGLEC1, IL1R2, | 0.892 | 0.884 | 0.901 | 0.927 | 0.871 | 0.982 | 0.949 | 0.886 | 1 |
| FAM20A, RETN | | | | | | | | | |
| IFI27, IFI44L, HERC6, IL1R2, OLAH, FAM20A, | 0.892 | 0.883 | 0.902 | 0.927 | 0.873 | 0.981 | 0.934 | 0.862 | 1 |
| RETN | | | | | | | | | |
| RSAD2, IFI27, IFIT1, SIGLEC1, ISG15, IL1R2, | 0.892 | 0.884 | 0.901 | 0.928 | 0.873 | 0.983 | 0.949 | 0.884 | 1 |
| FAM20A | | | | | | | | | |
| IFI27, OAS1, ISG15, HERC6, IL1R2, FAM20A, | 0.892 | 0.884 | 0.901 | 0.929 | 0.879 | 0.979 | 0.923 | 0.835 | 1 |
| MMP8 | | | | | | | | | |
| RSAD2, IFI27, HERC6, SLC1A2, OLAH, | 0.892 | 0.883 | 0.902 | 0.931 | 0.881 | 0.982 | 0.923 | 0.843 | 1 |
| FAM20A, MMP8 | | | | | | | | | |
| IFI27, OAS1, IL1R2, OLAH, FAM20A, RETN, | 0.892 | 0.883 | 0.901 | 0.929 | 0.875 | 0.984 | 0.930 | 0.858 | 1 |
| MMP8 | | | | | | | | | |
| IFI27, OAS1, IFI44L, SIGLEC1, OLAH, | 0.892 | 0.883 | 0.901 | 0.923 | 0.864 | 0.983 | 0.950 | 0.891 | 1 |
| FAM20A, RETN | | | | | | | | | |
| RSAD2, IFI27, SIGLEC1, ISG15, IL1R2, | 0.892 | 0.883 | 0.901 | 0.928 | 0.873 | 0.983 | 0.946 | 0.878 | 1 |
| FAM20A, RETN | | | | | | | | | |
| IFI27, OAS1, IFIT1, SIGLEC1, SLC1A2, OLAH, | 0.892 | 0.883 | 0.902 | 0.932 | 0.878 | 0.985 | 0.943 | 0.879 | 1 |
| FAM20A | | | | | | | | | |
| RSAD2, IFI27, IL1R2, OLAH, FAM20A, RETN, | 0.892 | 0.883 | 0.901 | 0.930 | 0.876 | 0.983 | 0.930 | 0.858 | 1 |
| MMP8 | | | | | | | | | |
| IFI27, OAS1, SIGLEC1, HERC6, OLAH, RETN, | 0.892 | 0.883 | 0.901 | 0.926 | 0.871 | 0.981 | 0.957 | 0.905 | 1 |
| MMP8 | | | | | | | | | |
| RSAD2, IFI27, IFI44L, SIGLEC1, ISG15, IL1R2, | 0.892 | 0.884 | 0.901 | 0.928 | 0.874 | 0.982 | 0.947 | 0.880 | 1 |
| FAM20A | | | | | | | | | |
| IFI27, IFI44L, SIGLEC1, ISG15, HERC6, | 0.892 | 0.883 | 0.901 | 0.923 | 0.866 | 0.980 | 0.942 | 0.866 | 1 |
| FAM20A, RETN | | | | | | | | | |
| RSAD2, IFI27, OAS1, SIGLEC1, IL1R2, | 0.892 | 0.883 | 0.901 | 0.928 | 0.872 | 0.984 | 0.950 | 0.886 | 1 |
| FAM20A, RETN | | | | | | | | | |
| RSAD2, IFI27, IFI44L, SIGLEC1, HERC6, | 0.892 | 0.883 | 0.901 | 0.928 | 0.874 | 0.982 | 0.939 | 0.864 | 1 |
| FAM20A, RETN | | | | | | | | | |
| RSAD2, IFI27, OAS1, HERC6, IL1R2, FAM20A, | 0.892 | 0.883 | 0.901 | 0.931 | 0.881 | 0.980 | 0.923 | 0.835 | 1 |
| MMP8 | | | | | | | | | |
| RSAD2, IFI27, SIGLEC1, ISG15, SLC1A2, | 0.892 | 0.883 | 0.901 | 0.929 | 0.876 | 0.982 | 0.936 | 0.866 | 1 |
| OLAH, FAM20A | | | | | | | | | |
| RSAD2, IFI27, OAS1, IFI44L, HERC6, IL1R2, | 0.892 | 0.884 | 0.901 | 0.927 | 0.876 | 0.978 | 0.926 | 0.842 | 1 |
| FAM20A | | | | | | | | | |
| IFI27, OAS1, IFI44L, HERC6, IL1R2, FAM20A, | 0.892 | 0.883 | 0.901 | 0.932 | 0.882 | 0.982 | 0.929 | 0.848 | 1 |
| RETN | | | | | | | | | |
| IFI27, IFI44L, HERC6, IL1R2, FAM20A, RETN, | 0.892 | 0.883 | 0.901 | 0.931 | 0.879 | 0.983 | 0.936 | 0.857 | 1 |
| MMP8 | | | | | | | | | |
| IFI27, SIGLEC1, ISG15, HERC6, IL1R2, | 0.892 | 0.883 | 0.901 | 0.928 | 0.874 | 0.983 | 0.949 | 0.881 | 1 |
| FAM20A, RETN | | | | | | | | | |
| IFI27, SIGLEC1, ISG15, HERC6, SLC1A2, | 0.892 | 0.882 | 0.902 | 0.931 | 0.879 | 0.983 | 0.939 | 0.872 | 1 |
| OLAH, FAM20A | | | | | | | | | |
| IFI27, OAS1, SIGLEC1, HERC6, SLC1A2, IL1R2, | 0.892 | 0.883 | 0.901 | 0.933 | 0.886 | 0.980 | 0.943 | 0.874 | 1 |
| FAM20A | | | | | | | | | |
| RSAD2, IFI27, IFIT1, IFI44L, HERC6, IL1R2, | 0.892 | 0.884 | 0.900 | 0.927 | 0.875 | 0.978 | 0.930 | 0.849 | 1 |
| FAM20A | | | | | | | | | |
| IFI27, OAS1, ISG15, HERC6, IL1R2, FAM20A, | 0.892 | 0.883 | 0.901 | 0.932 | 0.882 | 0.982 | 0.925 | 0.842 | 1 |
| RETN | | | | | | | | | |
| IFI27, IFIT1, SIGLEC1, ISG15, FAM20A, RETN, | 0.892 | 0.882 | 0.901 | 0.923 | 0.864 | 0.983 | 0.946 | 0.872 | 1 |
| MMP8 | | | | | | | | | |
| IFI27, IFIT1, IFI44L, HERC6, IL1R2, FAM20A, | 0.892 | 0.883 | 0.900 | 0.931 | 0.880 | 0.982 | 0.929 | 0.849 | 1 |
| RETN | | | | | | | | | |
| IFI27, ISG15, SLC1A2, IL1R2, OLAH, FAM20A, | 0.892 | 0.883 | 0.900 | 0.931 | 0.883 | 0.979 | 0.920 | 0.838 | 1 |
| MMP8 | | | | | | | | | |
| RSAD2, IFI27, OAS1, ISG15, OLAH, FAM20A, | 0.892 | 0.882 | 0.901 | 0.921 | 0.863 | 0.980 | 0.933 | 0.866 | 1.000 |
| MMP8 | | | | | | | | | |
| IFI27, SIGLEC1, HERC6, SLC1A2, OLAH, | 0.892 | 0.882 | 0.902 | 0.927 | 0.871 | 0.983 | 0.952 | 0.891 | 1 |
| FAM20A, MMP8 | | | | | | | | | |
| IFI27, IFI44L, ISG15, IL1R2, OLAH, FAM20A, | 0.892 | 0.883 | 0.901 | 0.923 | 0.865 | 0.980 | 0.930 | 0.859 | 1 |
| RETN | | | | | | | | | |
| IFI27, OAS1, IFIT1, HERC6, IL1R2, FAM20A, | 0.892 | 0.883 | 0.901 | 0.931 | 0.880 | 0.982 | 0.929 | 0.848 | 1 |
| RETN | | | | | | | | | |
| RSAD2, IFI27, IFI44L, ISG15, HERC6, IL1R2, | 0.892 | 0.883 | 0.900 | 0.928 | 0.877 | 0.979 | 0.928 | 0.845 | 1 |
| FAM20A | | | | | | | | | |
| IFI27, IFIT1, HERC6, IL1R2, FAM20A, RETN, | 0.892 | 0.882 | 0.901 | 0.931 | 0.878 | 0.983 | 0.936 | 0.857 | 1 |
| MMP8 | | | | | | | | | |
| IFI27, OAS1, SIGLEC1, HERC6, IL1R2, RETN, | 0.892 | 0.883 | 0.900 | 0.924 | 0.871 | 0.978 | 0.959 | 0.911 | 1 |
| MMP8 | | | | | | | | | |
| IFI27, IFIT1, IFI44L, SIGLEC1, ISG15, HERC6, | 0.892 | 0.883 | 0.900 | 0.920 | 0.860 | 0.980 | 0.942 | 0.867 | 1 |
| FAM20A | | | | | | | | | |
| IFI27, OAS1, IFIT1, SIGLEC1, ISG15, HERC6, | 0.892 | 0.883 | 0.900 | 0.920 | 0.863 | 0.976 | 0.942 | 0.884 | 1 |
| RETN | | | | | | | | | |
| IFI27, OAS1, IFIT1, ISG15, OLAH, FAM20A, | 0.892 | 0.882 | 0.901 | 0.921 | 0.861 | 0.981 | 0.936 | 0.871 | 1 |
| MMP8 | | | | | | | | | |
| RSAD2, IFI27, OAS1, IFI44L, OLAH, FAM20A, | 0.892 | 0.882 | 0.902 | 0.923 | 0.864 | 0.982 | 0.935 | 0.869 | 1 |
| MMP8 | | | | | | | | | |
| RSAD2, IFI27, OAS1, HERC6, IL1R2, FAM20A, | 0.892 | 0.883 | 0.900 | 0.930 | 0.879 | 0.981 | 0.929 | 0.848 | 1 |
| RETN | | | | | | | | | |
| IFIT1, IFI44L, SIGLEC1, HERC6, IL1R2, | 0.892 | 0.883 | 0.900 | 0.924 | 0.870 | 0.978 | 0.962 | 0.906 | 1 |
| FAM20A, MMP8 | | | | | | | | | |
| IFI27, OAS1, SIGLEC1, SLC1A2, IL1R2, OLAH, | 0.891 | 0.882 | 0.900 | 0.930 | 0.877 | 0.982 | 0.941 | 0.876 | 1 |
| FAM20A | | | | | | | | | |
| IFI27, SIGLEC1, HERC6, SLC1A2, IL1R2, | 0.891 | 0.882 | 0.901 | 0.932 | 0.884 | 0.980 | 0.955 | 0.887 | 1 |
| FAM20A, MMP8 | | | | | | | | | |
| IFI27, IFIT1, IFI44L, SIGLEC1, FAM20A, RETN, | 0.891 | 0.882 | 0.901 | 0.923 | 0.864 | 0.982 | 0.949 | 0.878 | 1 |
| MMP8 | | | | | | | | | |
| IFI27, OAS1, IFI44L, ISG15, OLAH, FAM20A, | 0.891 | 0.882 | 0.901 | 0.921 | 0.862 | 0.981 | 0.936 | 0.870 | 1 |
| MMP8 | | | | | | | | | |
| OAS1, IFIT1, SIGLEC1, HERC6, IL1R2, | 0.891 | 0.883 | 0.900 | 0.923 | 0.868 | 0.977 | 0.962 | 0.906 | 1 |
| FAM20A, MMP8 | | | | | | | | | |
| IFI27, SIGLEC1, ISG15, SLC1A2, IL1R2, OLAH, | 0.891 | 0.882 | 0.900 | 0.933 | 0.884 | 0.982 | 0.938 | 0.870 | 1 |
| FAM20A | | | | | | | | | |
| IFI27, IFI44L, SIGLEC1, HERC6, FAM20A, | 0.891 | 0.882 | 0.901 | 0.924 | 0.866 | 0.982 | 0.950 | 0.877 | 1 |
| RETN, MMP8 | | | | | | | | | |
| IFI27, OAS1, IFIT1, SIGLEC1, FAM20A, RETN, | 0.891 | 0.882 | 0.901 | 0.923 | 0.864 | 0.982 | 0.948 | 0.879 | 1 |
| MMP8 | | | | | | | | | |
| IFI27, IFI44L, ISG15, HERC6, IL1R2, FAM20A, | 0.891 | 0.882 | 0.900 | 0.931 | 0.880 | 0.982 | 0.929 | 0.849 | 1 |
| RETN | | | | | | | | | |
| IFI27, IFI44L, SIGLEC1, HERC6, OLAH, RETN, | 0.891 | 0.882 | 0.900 | 0.924 | 0.868 | 0.980 | 0.953 | 0.902 | 1 |
| MMP8 | | | | | | | | | |
| RSAD2, IFI27, SIGLEC1, HERC6, SLC1A2, | 0.891 | 0.882 | 0.901 | 0.931 | 0.878 | 0.983 | 0.938 | 0.870 | 1 |
| OLAH, FAM20A | | | | | | | | | |
| RSAD2, IFI27, OAS1, IFIT1, OLAH, FAM20A, | 0.891 | 0.882 | 0.901 | 0.922 | 0.863 | 0.981 | 0.939 | 0.876 | 1 |
| MMP8 | | | | | | | | | |
| IFI27, SIGLEC1, HERC6, IL1R2, OLAH, | 0.891 | 0.882 | 0.901 | 0.925 | 0.868 | 0.982 | 0.949 | 0.886 | 1 |
| FAM20A, RETN | | | | | | | | | |
| IFI27, OAS1, IFIT1, HERC6, OLAH, RETN, | 0.891 | 0.882 | 0.900 | 0.924 | 0.867 | 0.980 | 0.945 | 0.886 | 1 |
| MMP8 | | | | | | | | | |
| IFI27, IFIT1, SIGLEC1, HERC6, OLAH, RETN, | 0.891 | 0.882 | 0.900 | 0.926 | 0.870 | 0.981 | 0.957 | 0.909 | 1 |
| MMP8 | | | | | | | | | |
| IFI27, IFIT1, SIGLEC1, HERC6, SLC1A2, IL1R2, | 0.891 | 0.882 | 0.900 | 0.936 | 0.890 | 0.981 | 0.946 | 0.876 | 1 |
| FAM20A | | | | | | | | | |
| IFI27, IFIT1, IFI44L, ISG15, HERC6, IL1R2, | 0.891 | 0.883 | 0.900 | 0.927 | 0.876 | 0.978 | 0.932 | 0.851 | 1 |
| FAM20A | | | | | | | | | |
| IFI27, IFIT1, SLC1A2, IL1R2, OLAH, FAM20A, | 0.891 | 0.882 | 0.900 | 0.931 | 0.881 | 0.981 | 0.923 | 0.842 | 1 |
| MMP8 | | | | | | | | | |
| IFI27, IFI44L, SIGLEC1, ISG15, SLC1A2, IL1R2, | 0.891 | 0.883 | 0.900 | 0.932 | 0.884 | 0.979 | 0.947 | 0.877 | 1 |
| FAM20A | | | | | | | | | |
| IFI27, OAS1, IFIT1, IFI44L, OLAH, FAM20A, | 0.891 | 0.882 | 0.901 | 0.921 | 0.861 | 0.981 | 0.937 | 0.871 | 1 |
| MMP8 | | | | | | | | | |
| IFI27, IFIT1, IFI44L, SIGLEC1, HERC6, OLAH, | 0.891 | 0.883 | 0.900 | 0.926 | 0.872 | 0.979 | 0.946 | 0.893 | 0.998 |
| RETN | | | | | | | | | |
| IFI27, SIGLEC1, SLC1A2, IL1R2, FAM20A, | 0.891 | 0.882 | 0.900 | 0.935 | 0.886 | 0.983 | 0.955 | 0.888 | 1 |
| RETN, MMP8 | | | | | | | | | |
| IFI27, IFIT1, IFI44L, ISG15, IL1R2, OLAH, | 0.891 | 0.883 | 0.900 | 0.924 | 0.867 | 0.981 | 0.932 | 0.861 | 1 |
| FAM20A | | | | | | | | | |
| RSAD2, IFI27, ISG15, HERC6, IL1R2, FAM20A, | 0.891 | 0.882 | 0.900 | 0.929 | 0.879 | 0.979 | 0.926 | 0.841 | 1 |
| MMP8 | | | | | | | | | |
| IFI27, OAS1, IFIT1, ISG15, HERC6, IL1R2, | 0.891 | 0.882 | 0.900 | 0.928 | 0.877 | 0.979 | 0.925 | 0.842 | 1 |
| FAM20A | | | | | | | | | |
| IFI27, OAS1, IFIT1, IFI44L, SIGLEC1, FAM20A, | 0.891 | 0.882 | 0.900 | 0.919 | 0.858 | 0.979 | 0.946 | 0.878 | 1 |
| RETN | | | | | | | | | |
| IFI27, IFI44L, SIGLEC1, SLC1A2, IL1R2, OLAH, | 0.891 | 0.882 | 0.900 | 0.930 | 0.878 | 0.982 | 0.947 | 0.882 | 1 |
| FAM20A | | | | | | | | | |
| RSAD2, IFI27, IFI44L, SIGLEC1, IL1R2, | 0.891 | 0.882 | 0.900 | 0.929 | 0.874 | 0.983 | 0.950 | 0.886 | 1 |
| FAM20A, RETN | | | | | | | | | |
| RSAD2, IFI27, IFIT1, SIGLEC1, SLC1A2, OLAH, | 0.891 | 0.882 | 0.900 | 0.930 | 0.877 | 0.983 | 0.942 | 0.878 | 1 |
| FAM20A | | | | | | | | | |
| IFI27, IFIT1, IFI44L, SLC1A2, OLAH, FAM20A, | 0.891 | 0.882 | 0.900 | 0.927 | 0.872 | 0.981 | 0.928 | 0.852 | 1 |
| MMP8 | | | | | | | | | |
| IFI27, OAS1, ISG15, HERC6, SLC1A2, OLAH, | 0.891 | 0.881 | 0.901 | 0.933 | 0.884 | 0.982 | 0.912 | 0.825 | 0.999 |
| FAM20A | | | | | | | | | |
| IFI27, OAS1, IFIT1, SIGLEC1, HERC6, IL1R2, | 0.891 | 0.883 | 0.899 | 0.925 | 0.873 | 0.977 | 0.945 | 0.889 | 1.000 |
| RETN | | | | | | | | | |
| RSAD2, IFI27, IFIT1, IFI44L, OLAH, FAM20A, | 0.891 | 0.881 | 0.900 | 0.922 | 0.862 | 0.982 | 0.936 | 0.870 | 1 |
| MMP8 | | | | | | | | | |
| RSAD2, IFI27, OAS1, ISG15, IL1R2, OLAH, | 0.891 | 0.882 | 0.900 | 0.924 | 0.869 | 0.980 | 0.926 | 0.853 | 0.999 |
| FAM20A | | | | | | | | | |
| RSAD2, IFI27, OAS1, IFIT1, HERC6, IL1R2, | 0.891 | 0.882 | 0.900 | 0.929 | 0.878 | 0.979 | 0.925 | 0.842 | 1 |
| FAM20A | | | | | | | | | |
| IFI27, OAS1, IFI44L, SIGLEC1, SLC1A2, OLAH, | 0.891 | 0.882 | 0.900 | 0.928 | 0.873 | 0.984 | 0.941 | 0.878 | 1 |
| FAM20A | | | | | | | | | |
| IFI27, IFIT1, SIGLEC1, SLC1A2, OLAH, | 0.891 | 0.882 | 0.900 | 0.929 | 0.875 | 0.984 | 0.943 | 0.879 | 1 |
| FAM20A, RETN | | | | | | | | | |
| RSAD2, IFI27, OAS1, HERC6, OLAH, RETN, | 0.891 | 0.882 | 0.900 | 0.923 | 0.867 | 0.980 | 0.945 | 0.886 | 1 |
| MMP8 | | | | | | | | | |
| RSAD2, IFI27, OAS1, IFIT1, SIGLEC1, HERC6, | 0.891 | 0.882 | 0.899 | 0.919 | 0.860 | 0.978 | 0.947 | 0.889 | 1 |
| RETN | | | | | | | | | |
| IFI27, IFI44L, SIGLEC1, HERC6, IL1R2, OLAH, | 0.891 | 0.883 | 0.899 | 0.926 | 0.878 | 0.975 | 0.943 | 0.892 | 0.995 |
| MMP8 | | | | | | | | | |
| RSAD2, IFI27, IFIT1, IFI44L, SIGLEC1, HERC6, | 0.891 | 0.882 | 0.899 | 0.923 | 0.865 | 0.981 | 0.942 | 0.872 | 1 |
| FAM20A | | | | | | | | | |
| IFI27, OAS1, IFI44L, ISG15, IL1R2, OLAH, | 0.891 | 0.882 | 0.900 | 0.924 | 0.867 | 0.981 | 0.928 | 0.856 | 1 |
| FAM20A | | | | | | | | | |
| IFI27, ISG15, HERC6, SLC1A2, OLAH, | 0.891 | 0.881 | 0.900 | 0.931 | 0.881 | 0.980 | 0.918 | 0.835 | 1 |
| FAM20A, MMP8 | | | | | | | | | |
| RSAD2, IFI27, SLC1A2, IL1R2, OLAH, | 0.891 | 0.882 | 0.899 | 0.930 | 0.879 | 0.980 | 0.922 | 0.841 | 1 |
| FAM20A, MMP8 | | | | | | | | | |
| RSAD2, IFI27, IFI44L, ISG15, OLAH, FAM20A, | 0.891 | 0.881 | 0.901 | 0.923 | 0.864 | 0.982 | 0.934 | 0.867 | 1 |
| MMP8 | | | | | | | | | |
| IFI27, OAS1, IFIT1, ISG15, IL1R2, OLAH, | 0.891 | 0.882 | 0.899 | 0.924 | 0.868 | 0.981 | 0.929 | 0.859 | 1.000 |
| FAM20A | | | | | | | | | |
| IFI27, OAS1, IFIT1, IFI44L, IL1R2, OLAH, | 0.891 | 0.882 | 0.899 | 0.925 | 0.867 | 0.982 | 0.932 | 0.860 | 1 |
| FAM20A | | | | | | | | | |
| IFI27, IFIT1, IFI44L, OLAH, FAM20A, RETN, | 0.891 | 0.881 | 0.900 | 0.924 | 0.864 | 0.983 | 0.941 | 0.875 | 1 |
| MMP8 | | | | | | | | | |
| RSAD2, IFI27, OAS1, HERC6, SLC1A2, OLAH, | 0.891 | 0.881 | 0.900 | 0.930 | 0.879 | 0.980 | 0.922 | 0.840 | 1 |
| FAM20A | | | | | | | | | |
| IFI27, OAS1, IFI44L, SIGLEC1, HERC6, OLAH, | 0.891 | 0.882 | 0.899 | 0.924 | 0.872 | 0.977 | 0.941 | 0.884 | 0.999 |
| RETN | | | | | | | | | |
| RSAD2, IFI27, OAS1, ISG15, HERC6, IL1R2, | 0.891 | 0.882 | 0.899 | 0.929 | 0.879 | 0.978 | 0.922 | 0.836 | 1 |
| FAM20A | | | | | | | | | |
| IFIT1, SIGLEC1, HERC6, IL1R2, OLAH, | 0.891 | 0.882 | 0.899 | 0.924 | 0.870 | 0.978 | 0.962 | 0.906 | 1 |
| FAM20A, MMP8 | | | | | | | | | |
| RSAD2, IFI27, SIGLEC1, HERC6, OLAH, RETN, | 0.891 | 0.882 | 0.900 | 0.924 | 0.869 | 0.980 | 0.955 | 0.906 | 1 |
| MMP8 | | | | | | | | | |
| IFI27, IFIT1, IFI44L, SIGLEC1, SLC1A2, IL1R2, | 0.891 | 0.882 | 0.899 | 0.934 | 0.887 | 0.981 | 0.943 | 0.874 | 1 |
| FAM20A | | | | | | | | | |
| IFI27, IFIT1, ISG15, SLC1A2, OLAH, FAM20A, | 0.891 | 0.881 | 0.900 | 0.926 | 0.872 | 0.981 | 0.924 | 0.847 | 1 |
| MMP8 | | | | | | | | | |
| IFI27, OAS1, IFIT1, SIGLEC1, ISG15, FAM20A, | 0.891 | 0.881 | 0.900 | 0.920 | 0.861 | 0.980 | 0.943 | 0.875 | 1 |
| RETN | | | | | | | | | |
| RSAD2, IFI27, HERC6, SLC1A2, IL1R2, OLAH, | 0.891 | 0.882 | 0.899 | 0.930 | 0.880 | 0.980 | 0.922 | 0.841 | 1 |
| FAM20A | | | | | | | | | |
| IFI27, OAS1, IFIT1, SIGLEC1, HERC6, FAM20A, | 0.890 | 0.882 | 0.899 | 0.923 | 0.866 | 0.980 | 0.938 | 0.867 | 1 |
| MMP8 | | | | | | | | | |
| IFI27, IFIT1, IFI44L, IL1R2, FAM20A, RETN, | 0.890 | 0.881 | 0.900 | 0.929 | 0.875 | 0.982 | 0.937 | 0.860 | 1 |
| MMP8 | | | | | | | | | |
| IFI27, IFIT1, ISG15, OLAH, FAM20A, RETN, | 0.890 | 0.881 | 0.900 | 0.921 | 0.861 | 0.981 | 0.940 | 0.874 | 1 |
| MMP8 | | | | | | | | | |
| IFI27, OAS1, IFIT1, IFI44L, SIGLEC1, HERC6, | 0.890 | 0.882 | 0.899 | 0.919 | 0.862 | 0.977 | 0.941 | 0.882 | 1 |
| RETN | | | | | | | | | |
| RSAD2, IFI27, ISG15, HERC6, IL1R2, FAM20A, | 0.890 | 0.882 | 0.899 | 0.931 | 0.881 | 0.982 | 0.930 | 0.850 | 1 |
| RETN | | | | | | | | | |
| IFI27, IFIT1, IFI44L, SIGLEC1, HERC6, | 0.890 | 0.882 | 0.899 | 0.921 | 0.862 | 0.980 | 0.939 | 0.864 | 1 |
| FAM20A, MMP8 | | | | | | | | | |
| RSAD2, IFI27, SIGLEC1, SLC1A2, IL1R2, | 0.890 | 0.881 | 0.900 | 0.929 | 0.876 | 0.982 | 0.946 | 0.882 | 1 |
| OLAH, FAM20A | | | | | | | | | |
| IFI27, OAS1, IFIT1, IFI44L, IL1R2, FAM20A, | 0.890 | 0.882 | 0.899 | 0.925 | 0.872 | 0.977 | 0.933 | 0.854 | 1 |
| MMP8 | | | | | | | | | |
| IFI27, IFI44L, ISG15, SLC1A2, IL1R2, OLAH, | 0.890 | 0.882 | 0.899 | 0.931 | 0.881 | 0.982 | 0.918 | 0.837 | 1.000 |
| FAM20A | | | | | | | | | |
| IFI27, OAS1, IFIT1, IFI44L, SIGLEC1, HERC6, | 0.890 | 0.882 | 0.899 | 0.922 | 0.865 | 0.980 | 0.939 | 0.868 | 1 |
| FAM20A | | | | | | | | | |
| IFI27, SIGLEC1, ISG15, SLC1A2, OLAH, | 0.890 | 0.881 | 0.900 | 0.930 | 0.877 | 0.983 | 0.936 | 0.866 | 1 |
| FAM20A, RETN | | | | | | | | | |
| IFI27, OAS1, IFIT1, IFI44L, ISG15, OLAH, | 0.890 | 0.881 | 0.899 | 0.920 | 0.860 | 0.981 | 0.935 | 0.867 | 1 |
| FAM20A | | | | | | | | | |
| IFI27, IFIT1, SIGLEC1, ISG15, SLC1A2, IL1R2, | 0.890 | 0.882 | 0.899 | 0.933 | 0.886 | 0.979 | 0.943 | 0.874 | 1 |
| FAM20A | | | | | | | | | |
| IFI27, IFI44L, SIGLEC1, ISG15, HERC6, IL1R2, | 0.890 | 0.882 | 0.898 | 0.926 | 0.877 | 0.975 | 0.941 | 0.888 | 0.995 |
| OLAH | | | | | | | | | |
| IFI27, IFIT1, SIGLEC1, ISG15, HERC6, | 0.890 | 0.882 | 0.899 | 0.920 | 0.860 | 0.979 | 0.939 | 0.864 | 1 |
| FAM20A, MMP8 | | | | | | | | | |
| IFI27, OAS1, IFI44L, SIGLEC1, ISG15, HERC6, | 0.890 | 0.882 | 0.898 | 0.920 | 0.863 | 0.978 | 0.943 | 0.883 | 1 |
| RETN | | | | | | | | | |
| IFI27, OAS1, HERC6, IL1R2, OLAH, RETN, | 0.890 | 0.881 | 0.899 | 0.923 | 0.868 | 0.979 | 0.945 | 0.886 | 1 |
| MMP8 | | | | | | | | | |
| RSAD2, IFI27, IFI44L, SIGLEC1, OLAH, | 0.890 | 0.881 | 0.900 | 0.924 | 0.864 | 0.983 | 0.950 | 0.889 | 1 |
| FAM20A, RETN | | | | | | | | | |
| IFI27, IFI44L, SIGLEC1, ISG15, HERC6, OLAH, | 0.890 | 0.882 | 0.899 | 0.923 | 0.867 | 0.978 | 0.946 | 0.893 | 0.999 |
| RETN | | | | | | | | | |
| RSAD2, IFI27, IFIT1, IFI44L, IL1R2, FAM20A, | 0.890 | 0.882 | 0.899 | 0.926 | 0.873 | 0.979 | 0.935 | 0.857 | 1 |
| MMP8 | | | | | | | | | |
| IFI27, IFI44L, ISG15, IL1R2, FAM20A, RETN, | 0.890 | 0.881 | 0.899 | 0.928 | 0.875 | 0.981 | 0.929 | 0.848 | 1 |
| MMP8 | | | | | | | | | |
| RSAD2, IFI27, OAS1, IFIT1, IL1R2, FAM20A, | 0.890 | 0.881 | 0.899 | 0.926 | 0.873 | 0.978 | 0.933 | 0.854 | 1 |
| MMP8 | | | | | | | | | |
| RSAD2, IFI27, ISG15, SLC1A2, OLAH, | 0.890 | 0.881 | 0.899 | 0.928 | 0.875 | 0.980 | 0.918 | 0.837 | 1 |
| FAM20A, MMP8 | | | | | | | | | |
| RSAD2, IFI27, OAS1, IFIT1, IL1R2, OLAH, | 0.890 | 0.882 | 0.899 | 0.924 | 0.867 | 0.981 | 0.933 | 0.863 | 1 |
| FAM20A | | | | | | | | | |
| RSAD2, IFI27, SIGLEC1, HERC6, SLC1A2, | 0.890 | 0.881 | 0.900 | 0.933 | 0.884 | 0.981 | 0.946 | 0.876 | 1 |
| IL1R2, FAM20A | | | | | | | | | |
| RSAD2, IFI27, IFIT1, ISG15, OLAH, FAM20A, | 0.890 | 0.881 | 0.900 | 0.921 | 0.861 | 0.981 | 0.937 | 0.872 | 1 |
| MMP8 | | | | | | | | | |
| IFI27, HERC6, SLC1A2, IL1R2, OLAH, | 0.890 | 0.881 | 0.899 | 0.933 | 0.885 | 0.982 | 0.925 | 0.845 | 1 |
| FAM20A, MMP8 | | | | | | | | | |
| IFI27, IFIT1, ISG15, HERC6, IL1R2, FAM20A, | 0.890 | 0.881 | 0.899 | 0.929 | 0.878 | 0.979 | 0.935 | 0.855 | 1 |
| MMP8 | | | | | | | | | |
| IFI27, OAS1, SIGLEC1, HERC6, IL1R2, OLAH, | 0.890 | 0.882 | 0.898 | 0.924 | 0.874 | 0.973 | 0.945 | 0.893 | 0.996 |
| MMP8 | | | | | | | | | |
| RSAD2, IFI27, IFIT1, HERC6, IL1R2, FAM20A, | 0.890 | 0.881 | 0.899 | 0.932 | 0.881 | 0.983 | 0.929 | 0.849 | 1 |
| RETN | | | | | | | | | |
| IFI27, OAS1, IFIT1, SIGLEC1, HERC6, RETN, | 0.890 | 0.881 | 0.899 | 0.919 | 0.861 | 0.977 | 0.946 | 0.888 | 1 |
| MMP8 | | | | | | | | | |
| IFI27, OAS1, IFIT1, HERC6, SLC1A2, OLAH, | 0.890 | 0.880 | 0.900 | 0.929 | 0.878 | 0.980 | 0.920 | 0.838 | 1 |
| FAM20A | | | | | | | | | |
| IFI27, OAS1, IFIT1, IL1R2, FAM20A, RETN, | 0.890 | 0.881 | 0.899 | 0.929 | 0.875 | 0.982 | 0.936 | 0.859 | 1 |
| MMP8 | | | | | | | | | |
| IFI27, IFIT1, ISG15, IL1R2, OLAH, FAM20A, | 0.890 | 0.881 | 0.899 | 0.923 | 0.866 | 0.980 | 0.932 | 0.861 | 1 |
| RETN | | | | | | | | | |
| IFIT1, SIGLEC1, ISG15, HERC6, IL1R2, | 0.890 | 0.881 | 0.899 | 0.922 | 0.868 | 0.977 | 0.962 | 0.906 | 1 |
| FAM20A, MMP8 | | | | | | | | | |
| RSAD2, IFI27, OAS1, SIGLEC1, SLC1A2, | 0.890 | 0.881 | 0.899 | 0.929 | 0.874 | 0.984 | 0.941 | 0.877 | 1 |
| OLAH, FAM20A | | | | | | | | | |
| IFI27, IFIT1, IFI44L, HERC6, SLC1A2, OLAH, | 0.890 | 0.880 | 0.899 | 0.930 | 0.877 | 0.982 | 0.923 | 0.842 | 1 |
| FAM20A | | | | | | | | | |
| IFI27, OAS1, IFIT1, SIGLEC1, HERC6, OLAH, | 0.890 | 0.881 | 0.898 | 0.923 | 0.871 | 0.974 | 0.938 | 0.881 | 0.995 |
| MMP8 | | | | | | | | | |
| RSAD2, IFI27, IFI44L, SIGLEC1, SLC1A2, | 0.890 | 0.881 | 0.899 | 0.927 | 0.870 | 0.983 | 0.943 | 0.879 | 1 |
| OLAH, FAM20A | | | | | | | | | |
| RSAD2, IFI27, ISG15, HERC6, SLC1A2, OLAH, | 0.890 | 0.880 | 0.899 | 0.931 | 0.881 | 0.982 | 0.915 | 0.832 | 0.999 |
| FAM20A | | | | | | | | | |
| IFI27, OAS1, HERC6, SLC1A2, IL1R2, OLAH, | 0.890 | 0.881 | 0.899 | 0.932 | 0.884 | 0.981 | 0.916 | 0.830 | 1 |
| FAM20A | | | | | | | | | |
| RSAD2, IFI27, IFI44L, ISG15, IL1R2, OLAH, | 0.890 | 0.881 | 0.899 | 0.923 | 0.866 | 0.980 | 0.928 | 0.856 | 1 |
| FAM20A | | | | | | | | | |
| IFI27, OAS1, ISG15, OLAH, FAM20A, RETN, | 0.890 | 0.880 | 0.899 | 0.923 | 0.864 | 0.982 | 0.939 | 0.872 | 1 |
| MMP8 | | | | | | | | | |
| IFI27, OAS1, SIGLEC1, SLC1A2, OLAH, | 0.890 | 0.881 | 0.899 | 0.927 | 0.871 | 0.983 | 0.940 | 0.875 | 1 |
| FAM20A, RETN | | | | | | | | | |
| IFI27, OAS1, IFIT1, IFI44L, HERC6, FAM20A, | 0.890 | 0.881 | 0.899 | 0.923 | 0.865 | 0.981 | 0.932 | 0.852 | 1 |
| RETN | | | | | | | | | |
| IFI27, OAS1, IFI44L, HERC6, SLC1A2, OLAH, | 0.890 | 0.880 | 0.900 | 0.930 | 0.879 | 0.981 | 0.922 | 0.842 | 1 |
| FAM20A | | | | | | | | | |
| IFI27, OAS1, ISG15, IL1R2, OLAH, FAM20A, | 0.890 | 0.881 | 0.899 | 0.925 | 0.870 | 0.981 | 0.927 | 0.855 | 0.999 |
| RETN | | | | | | | | | |
| IFI27, IFI44L, SIGLEC1, HERC6, SLC1A2, | 0.890 | 0.880 | 0.899 | 0.928 | 0.873 | 0.983 | 0.949 | 0.885 | 1 |
| OLAH, FAM20A | | | | | | | | | |
| IFI27, OAS1, IFI44L, ISG15, HERC6, FAM20A, | 0.890 | 0.880 | 0.899 | 0.925 | 0.868 | 0.982 | 0.934 | 0.855 | 1 |
| RETN | | | | | | | | | |
| IFI27, IFIT1, SIGLEC1, ISG15, HERC6, OLAH, | 0.890 | 0.881 | 0.899 | 0.923 | 0.86 | 0.978 | 0.946 | 0.893 | 0.999 |
| RETN | | | | | | | | | |
| IFI27, OAS1, IFIT1, SIGLEC1, SLC1A2, IL1R2, | 0.890 | 0.881 | 0.899 | 0.932 | 0.883 | 0.980 | 0.943 | 0.875 | 1 |
| FAM20A | | | | | | | | | |
| IFI27, OAS1, ISG15, HERC6, OLAH, RETN, | 0.890 | 0.881 | 0.899 | 0.924 | 0.869 | 0.980 | 0.945 | 0.886 | 1 |
| MMP8 | | | | | | | | | |
| RSAD2, IFIT1, SIGLEC1, HERC6, IL1R2, | 0.890 | 0.881 | 0.898 | 0.923 | 0.869 | 0.977 | 0.962 | 0.906 | 1 |
| FAM20A, MMP8 | | | | | | | | | |
| RSAD2, IFI27, HERC6, IL1R2, OLAH, RETN, | 0.890 | 0.881 | 0.899 | 0.925 | 0.870 | 0.980 | 0.943 | 0.887 | 1 |
| MMP8 | | | | | | | | | |
| IFI27, IFIT1, ISG15, HERC6, IL1R2, FAM20A, | 0.890 | 0.881 | 0.899 | 0.931 | 0.880 | 0.982 | 0.929 | 0.849 | 1 |
| RETN | | | | | | | | | |
| RSAD2, IFI27, IFI44L, HERC6, IL1R2, FAM20A, | 0.890 | 0.880 | 0.899 | 0.932 | 0.882 | 0.983 | 0.929 | 0.849 | 1 |
| RETN | | | | | | | | | |
| RSAD2, IFI27, OAS1, SIGLEC1, HERC6, OLAH, | 0.890 | 0.881 | 0.898 | 0.924 | 0.868 | 0.979 | 0.946 | 0.889 | 1 |
| RETN | | | | | | | | | |
| IFI27, OAS1, IFI44L, SIGLEC1, HERC6, IL1R2, | 0.890 | 0.882 | 0.898 | 0.925 | 0.874 | 0.976 | 0.940 | 0.885 | 0.996 |
| RETN | | | | | | | | | |
| RSAD2, IFI27, OAS1, OLAH, FAM20A, RETN, | 0.890 | 0.880 | 0.899 | 0.922 | 0.864 | 0.981 | 0.940 | 0.874 | 1 |
| MMP8 | | | | | | | | | |
| RSAD2, IFI27, OAS1, IFIT1, IFI44L, OLAH, | 0.890 | 0.880 | 0.899 | 0.921 | 0.861 | 0.981 | 0.933 | 0.863 | 1 |
| FAM20A | | | | | | | | | |
| IFI27, OAS1, IFIT1, OLAH, FAM20A, RETN, | 0.890 | 0.880 | 0.899 | 0.923 | 0.863 | 0.982 | 0.942 | 0.877 | 1 |
| MMP8 | | | | | | | | | |
| IFI27, OAS1, SIGLEC1, ISG15, HERC6, RETN, | 0.890 | 0.880 | 0.899 | 0.922 | 0.863 | 0.980 | 0.949 | 0.889 | 1 |
| MMP8 | | | | | | | | | |
| RSAD2, IFI27, OAS1, SLC1A2, OLAH, FAM20A, | 0.890 | 0.880 | 0.899 | 0.925 | 0.869 | 0.981 | 0.923 | 0.843 | 1 |
| MMP8 | | | | | | | | | |
| RSAD2, IFI27, IFIT1, IFI44L, IL1R2, OLAH, | 0.890 | 0.881 | 0.898 | 0.925 | 0.868 | 0.982 | 0.930 | 0.858 | 1 |
| FAM20A | | | | | | | | | |
| RSAD2, IFI27, OAS1, SIGLEC1, HERC6, IL1R2, | 0.890 | 0.881 | 0.898 | 0.923 | 0.869 | 0.977 | 0.954 | 0.901 | 1 |
| RETN | | | | | | | | | |
| RSAD2, IFI27, IFI44L, SLC1A2, OLAH, | 0.890 | 0.880 | 0.899 | 0.930 | 0.876 | 0.983 | 0.928 | 0.854 | 1 |
| FAM20A, MMP8 | | | | | | | | | |
| IFI27, IFIT1, IFI44L, ISG15, OLAH, FAM20A, | 0.890 | 0.881 | 0.899 | 0.920 | 0.860 | 0.981 | 0.935 | 0.865 | 1 |
| RETN | | | | | | | | | |
| IFI27, IFIT1, IFI44L, IL1R2, OLAH, FAM20A, | 0.890 | 0.881 | 0.898 | 0.924 | 0.867 | 0.981 | 0.930 | 0.858 | 1 |
| RETN | | | | | | | | | |
| RSAD2, IFI27, IFIT1, SIGLEC1, ISG15, HERC6, | 0.890 | 0.881 | 0.898 | 0.922 | 0.865 | 0.980 | 0.941 | 0.871 | 1 |
| FAM20A | | | | | | | | | |
| IFI27, OAS1, SIGLEC1, HERC6, IL1R2, OLAH, | 0.889 | 0.881 | 0.898 | 0.923 | 0.868 | 0.978 | 0.945 | 0.889 | 1 |
| RETN | | | | | | | | | |
| RSAD2, IFI27, IFI44L, SIGLEC1, HERC6, IL1R2, | 0.889 | 0.881 | 0.898 | 0.927 | 0.879 | 0.976 | 0.942 | 0.890 | 0.994 |
| OLAH | | | | | | | | | |
| IFI27, IFI44L, ISG15, HERC6, SLC1A2, OLAH, | 0.889 | 0.880 | 0.899 | 0.930 | 0.879 | 0.982 | 0.918 | 0.837 | 1 |
| FAM20A | | | | | | | | | |
| IFI27, OAS1, SLC1A2, IL1R2, OLAH, FAM20A, | 0.889 | 0.881 | 0.898 | 0.931 | 0.881 | 0.980 | 0.924 | 0.845 | 1 |
| MMP8 | | | | | | | | | |
| RSAD2, IFI27, OAS1, SIGLEC1, HERC6, IL1R2, | 0.889 | 0.881 | 0.898 | 0.924 | 0.875 | 0.974 | 0.942 | 0.890 | 0.995 |
| OLAH | | | | | | | | | |
| RSAD2, IFI27, IFIT1, HERC6, OLAH, RETN, | 0.889 | 0.880 | 0.899 | 0.925 | 0.869 | 0.981 | 0.943 | 0.886 | 1 |
| MMP8 | | | | | | | | | |
| RSAD2, IFI27, SIGLEC1, HERC6, IL1R2, OLAH, | 0.889 | 0.881 | 0.898 | 0.926 | 0.877 | 0.975 | 0.942 | 0.890 | 0.995 |
| MMP8 | | | | | | | | | |
| IFI27, OAS1, IFIT1, ISG15, HERC6, FAM20A, | 0.889 | 0.880 | 0.899 | 0.923 | 0.867 | 0.980 | 0.935 | 0.857 | 1 |
| RETN | | | | | | | | | |
| IFI27, IFIT1, IFI44L, ISG15, SLC1A2, OLAH, | 0.889 | 0.880 | 0.898 | 0.928 | 0.874 | 0.982 | 0.916 | 0.834 | 0.999 |
| FAM20A | | | | | | | | | |
| RSAD2, IFI27, OAS1, SIGLEC1, FAM20A, | 0.889 | 0.879 | 0.900 | 0.925 | 0.867 | 0.984 | 0.947 | 0.875 | 1 |
| RETN, MMP8 | | | | | | | | | |
| RSAD2, IFI27, OAS1, SIGLEC1, ISG15, HERC6, | 0.889 | 0.881 | 0.898 | 0.920 | 0.862 | 0.979 | 0.948 | 0.887 | 1 |
| RETN | | | | | | | | | |
| IFI27, OAS1, SIGLEC1, ISG15, HERC6, OLAH, | 0.889 | 0.880 | 0.898 | 0.923 | 0.867 | 0.978 | 0.945 | 0.887 | 1 |
| RETN | | | | | | | | | |
| IFI27, OAS1, IFI44L, SIGLEC1, SLC1A2, IL1R2, | 0.889 | 0.880 | 0.898 | 0.931 | 0.881 | 0.980 | 0.942 | 0.872 | 1 |
| FAM20A | | | | | | | | | |
| RSAD2, IFI27, OAS1, IFIT1, SIGLEC1, HERC6, | 0.889 | 0.881 | 0.898 | 0.923 | 0.866 | 0.980 | 0.939 | 0.868 | 1 |
| FAM20A | | | | | | | | | |
| IFI27, OAS1, IFIT1, SIGLEC1, ISG15, HERC6, | 0.889 | 0.881 | 0.898 | 0.923 | 0.867 | 0.980 | 0.938 | 0.865 | 1 |
| FAM20A | | | | | | | | | |
| RSAD2, IFI27, ISG15, IL1R2, OLAH, FAM20A, | 0.889 | 0.880 | 0.898 | 0.924 | 0.868 | 0.981 | 0.927 | 0.853 | 1 |
| RETN | | | | | | | | | |
| IFI27, IFI44L, SIGLEC1, ISG15, HERC6, OLAH, | 0.889 | 0.881 | 0.898 | 0.925 | 0.873 | 0.977 | 0.945 | 0.892 | 0.997 |
| MMP8 | | | | | | | | | |
| IFI27, OAS1, IFI44L, SIGLEC1, HERC6, IL1R2, | 0.889 | 0.881 | 0.898 | 0.927 | 0.878 | 0.975 | 0.946 | 0.895 | 0.996 |
| OLAH | | | | | | | | | |
| RSAD2, IFI27, IFI44L, SIGLEC1, HERC6, | 0.889 | 0.881 | 0.898 | 0.923 | 0.869 | 0.977 | 0.948 | 0.896 | 1.000 |
| OLAH, RETN | | | | | | | | | |
| IFI27, IFIT1, IFI44L, ISG15, HERC6, FAM20A, | 0.889 | 0.880 | 0.898 | 0.921 | 0.863 | 0.979 | 0.933 | 0.853 | 1 |
| RETN | | | | | | | | | |
| IFI27, OAS1, IFI44L, SIGLEC1, HERC6, OLAH, | 0.889 | 0.881 | 0.898 | 0.926 | 0.875 | 0.977 | 0.943 | 0.891 | 0.996 |
| MMP8 | | | | | | | | | |
| RSAD2, IFI27, IFIT1, ISG15, HERC6, IL1R2, | 0.889 | 0.881 | 0.898 | 0.927 | 0.876 | 0.978 | 0.930 | 0.850 | 1 |
| FAM20A | | | | | | | | | |
| IFI27, IFI44L, SIGLEC1, HERC6, SLC1A2, | 0.889 | 0.880 | 0.898 | 0.931 | 0.882 | 0.980 | 0.947 | 0.876 | 1 |
| IL1R2, FAM20A | | | | | | | | | |
| IFI27, OAS1, IFIT1, ISG15, HERC6, OLAH, | 0.889 | 0.880 | 0.898 | 0.921 | 0.866 | 0.977 | 0.938 | 0.876 | 1.000 |
| RETN | | | | | | | | | |
| IFI27, IFI44L, ISG15, HERC6, OLAH, RETN, | 0.889 | 0.880 | 0.898 | 0.922 | 0.864 | 0.980 | 0.948 | 0.895 | 1 |
| MMP8 | | | | | | | | | |
| RSAD2, IFI27, OAS1, SIGLEC1, HERC6, OLAH, | 0.889 | 0.880 | 0.898 | 0.923 | 0.870 | 0.975 | 0.938 | 0.881 | 0.995 |
| MMP8 | | | | | | | | | |
| RSAD2, IFI27, IFIT1, ISG15, IL1R2, OLAH, | 0.889 | 0.880 | 0.898 | 0.924 | 0.867 | 0.981 | 0.932 | 0.861 | 1 |
| FAM20A | | | | | | | | | |
| IFI27, SLC1A2, IL1R2, OLAH, FAM20A, RETN, | 0.889 | 0.880 | 0.899 | 0.933 | 0.883 | 0.982 | 0.922 | 0.842 | 1 |
| MMP8 | | | | | | | | | |
| IFI27, OAS1, ISG15, SLC1A2, OLAH, FAM20A, | 0.889 | 0.880 | 0.898 | 0.927 | 0.875 | 0.980 | 0.920 | 0.841 | 0.998 |
| MMP8 | | | | | | | | | |
| OAS1, IFIT1, SIGLEC1, IL1R2, OLAH, FAM20A, | 0.889 | 0.881 | 0.898 | 0.919 | 0.861 | 0.977 | 0.965 | 0.913 | 1 |
| MMP8 | | | | | | | | | |
| RSAD2, IFI27, OAS1, IFI44L, IL1R2, OLAH, | 0.889 | 0.880 | 0.898 | 0.924 | 0.867 | 0.982 | 0.930 | 0.858 | 1 |
| FAM20A | | | | | | | | | |
| IFI27, OAS1, SIGLEC1, ISG15, FAM20A, RETN, | 0.889 | 0.879 | 0.899 | 0.923 | 0.863 | 0.984 | 0.945 | 0.871 | 1 |
| MMP8 | | | | | | | | | |
| RSAD2, IFI27, OAS1, IFIT1, HERC6, OLAH, | 0.889 | 0.880 | 0.898 | 0.923 | 0.867 | 0.979 | 0.940 | 0.880 | 1 |
| RETN | | | | | | | | | |
| RSAD2, IFI27, IFIT1, SLC1A2, OLAH, FAM20A, | 0.889 | 0.880 | 0.898 | 0.926 | 0.870 | 0.981 | 0.929 | 0.857 | 1 |
| MMP8 | | | | | | | | | |
| IFI27, IFIT1, IFI44L, ISG15, IL1R2, FAM20A, | 0.889 | 0.880 | 0.898 | 0.926 | 0.873 | 0.978 | 0.933 | 0.854 | 1 |
| MMP8 | | | | | | | | | |
| IFI27, OAS1, IFIT1, SIGLEC1, HERC6, IL1R2, | 0.889 | 0.881 | 0.897 | 0.924 | 0.875 | 0.974 | 0.942 | 0.890 | 0.995 |
| OLAH | | | | | | | | | |
| IFI27, IFIT1, IFI44L, HERC6, OLAH, RETN, | 0.889 | 0.880 | 0.898 | 0.922 | 0.864 | 0.979 | 0.949 | 0.897 | 1 |
| MMP8 | | | | | | | | | |
| IFI27, OAS1, IFIT1, IL1R2, OLAH, FAM20A, | 0.889 | 0.880 | 0.898 | 0.925 | 0.869 | 0.981 | 0.932 | 0.861 | 1 |
| RETN | | | | | | | | | |
| IFI27, ISG15, HERC6, SLC1A2, IL1R2, OLAH, | 0.889 | 0.880 | 0.898 | 0.932 | 0.883 | 0.981 | 0.917 | 0.834 | 1 |
| FAM20A | | | | | | | | | |
| IFI27, SIGLEC1, HERC6, SLC1A2, IL1R2, | 0.889 | 0.880 | 0.898 | 0.927 | 0.872 | 0.981 | 0.943 | 0.875 | 1 |
| OLAH, FAM20A | | | | | | | | | |
| IFI27, IFI44L, SIGLEC1, HERC6, IL1R2, OLAH, | 0.889 | 0.880 | 0.898 | 0.923 | 0.870 | 0.977 | 0.948 | 0.896 | 0.999 |
| RETN | | | | | | | | | |
| RSAD2, IFI27, IFIT1, OLAH, FAM20A, RETN, | 0.889 | 0.879 | 0.899 | 0.923 | 0.864 | 0.982 | 0.940 | 0.875 | 1 |
| MMP8 | | | | | | | | | |
| IFI27, IFIT1, ISG15, HERC6, SLC1A2, OLAH, | 0.889 | 0.879 | 0.899 | 0.930 | 0.879 | 0.982 | 0.916 | 0.834 | 0.999 |
| FAM20A | | | | | | | | | |
| RSAD2, IFI27, IFIT1, SIGLEC1, HERC6, OLAH, | 0.889 | 0.880 | 0.898 | 0.925 | 0.872 | 0.978 | 0.942 | 0.887 | 0.998 |
| RETN | | | | | | | | | |
| IFI27, IFI44L, SIGLEC1, ISG15, IL1R2, OLAH, | 0.889 | 0.881 | 0.897 | 0.924 | 0.875 | 0.974 | 0.943 | 0.891 | 0.996 |
| MMP8 | | | | | | | | | |
| RSAD2, IFI27, IFI44L, HERC6, SLC1A2, OLAH, | 0.889 | 0.879 | 0.899 | 0.930 | 0.878 | 0.981 | 0.923 | 0.844 | 1 |
| FAM20A | | | | | | | | | |
| RSAD2, IFI27, IFI44L, SIGLEC1, HERC6, | 0.889 | 0.881 | 0.897 | 0.924 | 0.871 | 0.977 | 0.947 | 0.897 | 0.997 |
| OLAH, MMP8 | | | | | | | | | |
| RSAD2, IFI27, OAS1, IFI44L, IL1R2, FAM20A, | 0.889 | 0.880 | 0.898 | 0.926 | 0.874 | 0.979 | 0.930 | 0.847 | 1 |
| MMP8 | | | | | | | | | |
| IFI27, OAS1, SIGLEC1, ISG15, HERC6, IL1R2, | 0.889 | 0.880 | 0.897 | 0.925 | 0.873 | 0.977 | 0.953 | 0.900 | 1 |
| RETN | | | | | | | | | |
| IFI27, OAS1, IFIT1, SLC1A2, OLAH, FAM20A, | 0.889 | 0.879 | 0.898 | 0.925 | 0.869 | 0.981 | 0.926 | 0.850 | 1 |
| MMP8 | | | | | | | | | |
| IFI27, OAS1, IFI44L, SLC1A2, OLAH, FAM20A, | 0.889 | 0.879 | 0.899 | 0.932 | 0.879 | 0.985 | 0.925 | 0.848 | 1 |
| MMP8 | | | | | | | | | |
| IFI27, IFIT1, HERC6, SLC1A2, OLAH, FAM20A, | 0.889 | 0.879 | 0.899 | 0.927 | 0.873 | 0.980 | 0.923 | 0.842 | 1 |
| MMP8 | | | | | | | | | |
| IFI27, OAS1, IFI44L, IL1R2, OLAH, FAM20A, | 0.889 | 0.880 | 0.898 | 0.924 | 0.866 | 0.981 | 0.929 | 0.856 | 1 |
| RETN | | | | | | | | | |
| RSAD2, IFI27, OAS1, IFI44L, ISG15, OLAH, | 0.889 | 0.880 | 0.898 | 0.921 | 0.861 | 0.981 | 0.930 | 0.861 | 1.000 |
| FAM20A | | | | | | | | | |
| IFI27, OAS1, IFIT1, IFI44L, SIGLEC1, HERC6, | 0.889 | 0.880 | 0.897 | 0.926 | 0.875 | 0.977 | 0.946 | 0.894 | 0.998 |
| OLAH | | | | | | | | | |
| IFI27, SIGLEC1, SLC1A2, OLAH, FAM20A, | 0.889 | 0.879 | 0.899 | 0.927 | 0.871 | 0.983 | 0.947 | 0.879 | 1 |
| RETN, MMP8 | | | | | | | | | |
| RSAD2, IFI27, OAS1, IFI44L, SIGLEC1, HERC6, | 0.889 | 0.880 | 0.897 | 0.919 | 0.860 | 0.977 | 0.941 | 0.875 | 1 |
| RETN | | | | | | | | | |
| IFI27, OAS1, IFIT1, IFI44L, HERC6, OLAH, | 0.889 | 0.880 | 0.897 | 0.923 | 0.868 | 0.978 | 0.938 | 0.877 | 0.999 |
| RETN | | | | | | | | | |
| IFI27, OAS1, IFIT1, HERC6, IL1R2, OLAH, | 0.889 | 0.880 | 0.897 | 0.922 | 0.867 | 0.978 | 0.939 | 0.878 | 1.000 |
| RETN | | | | | | | | | |
| RSAD2, IFI27, IFIT1, IL1R2, FAM20A, RETN, | 0.889 | 0.880 | 0.898 | 0.930 | 0.877 | 0.982 | 0.934 | 0.856 | 1 |
| MMP8 | | | | | | | | | |
| IFI27, OAS1, HERC6, SLC1A2, OLAH, FAM20A, | 0.889 | 0.879 | 0.898 | 0.932 | 0.883 | 0.981 | 0.916 | 0.830 | 1 |
| RETN | | | | | | | | | |
| IFI27, IFIT1, IFI44L, SIGLEC1, ISG15, HERC6, | 0.889 | 0.880 | 0.897 | 0.925 | 0.873 | 0.978 | 0.945 | 0.892 | 0.997 |
| OLAH | | | | | | | | | |
| IFI27, OAS1, IFI44L, HERC6, OLAH, RETN, | 0.889 | 0.879 | 0.898 | 0.923 | 0.867 | 0.980 | 0.941 | 0.883 | 1.000 |
| MMP8 | | | | | | | | | |
| RSAD2, IFI27, OAS1, IL1R2, OLAH, FAM20A, | 0.889 | 0.880 | 0.898 | 0.924 | 0.867 | 0.981 | 0.930 | 0.858 | 1 |
| RETN | | | | | | | | | |
| IFI27, IFIT1, SIGLEC1, HERC6, IL1R2, OLAH, | 0.889 | 0.880 | 0.897 | 0.923 | 0.869 | 0.978 | 0.948 | 0.896 | 1 |
| RETN | | | | | | | | | |
| RSAD2, IFI27, IFIT1, IFI44L, ISG15, OLAH, | 0.889 | 0.879 | 0.898 | 0.920 | 0.860 | 0.981 | 0.933 | 0.863 | 1 |
| FAM20A | | | | | | | | | |
| RSAD2, IFI27, ISG15, OLAH, FAM20A, RETN, | 0.889 | 0.879 | 0.898 | 0.922 | 0.863 | 0.981 | 0.940 | 0.874 | 1 |
| MMP8 | | | | | | | | | |
| RSAD2, IFI27, SIGLEC1, ISG15, SLC1A2, | 0.889 | 0.879 | 0.898 | 0.933 | 0.885 | 0.980 | 0.945 | 0.874 | 1 |
| IL1R2, FAM20A | | | | | | | | | |
| RSAD2, IFI27, OAS1, HERC6, IL1R2, OLAH, | 0.889 | 0.880 | 0.897 | 0.922 | 0.866 | 0.978 | 0.942 | 0.883 | 1 |
| RETN | | | | | | | | | |
| IFI27, IFI44L, HERC6, IL1R2, OLAH, RETN, | 0.888 | 0.880 | 0.897 | 0.922 | 0.866 | 0.979 | 0.947 | 0.893 | 1 |
| MMP8 | | | | | | | | | |
| IFI27, OAS1, IFI44L, ISG15, SLC1A2, OLAH, | 0.888 | 0.879 | 0.898 | 0.930 | 0.877 | 0.983 | 0.918 | 0.837 | 0.999 |
| FAM20A | | | | | | | | | |
| RSAD2, IFI27, OAS1, HERC6, IL1R2, OLAH, | 0.888 | 0.880 | 0.897 | 0.927 | 0.879 | 0.976 | 0.938 | 0.880 | 0.996 |
| MMP8 | | | | | | | | | |
| RSAD2, IFI27, IFIT1, SIGLEC1, FAM20A, | 0.888 | 0.879 | 0.898 | 0.924 | 0.866 | 0.982 | 0.947 | 0.877 | 1 |
| RETN, MMP8 | | | | | | | | | |
| IFI27, IFIT1, SIGLEC1, SLC1A2, IL1R2, | 0.888 | 0.879 | 0.897 | 0.934 | 0.886 | 0.981 | 0.943 | 0.874 | 1 |
| FAM20A, RETN | | | | | | | | | |
| RSAD2, IFI27, IFIT1, HERC6, SLC1A2, OLAH, | 0.888 | 0.879 | 0.898 | 0.928 | 0.876 | 0.980 | 0.922 | 0.840 | 1 |
| FAM20A | | | | | | | | | |
| IFI27, OAS1, SIGLEC1, ISG15, HERC6, IL1R2, | 0.888 | 0.880 | 0.897 | 0.924 | 0.874 | 0.974 | 0.940 | 0.885 | 0.996 |
| OLAH | | | | | | | | | |
| IFI27, IFIT1, IFI44L, SIGLEC1, HERC6, IL1R2, | 0.888 | 0.880 | 0.897 | 0.927 | 0.878 | 0.976 | 0.943 | 0.892 | 0.995 |
| OLAH | | | | | | | | | |
| IFI27, OAS1, IFIT1, IFI44L, SLC1A2, OLAH, | 0.888 | 0.879 | 0.898 | 0.929 | 0.874 | 0.984 | 0.924 | 0.846 | 1 |
| FAM20A | | | | | | | | | |
| IFI27, OAS1, SIGLEC1, ISG15, SLC1A2, IL1R2, | 0.888 | 0.879 | 0.898 | 0.933 | 0.886 | 0.980 | 0.945 | 0.876 | 1 |
| FAM20A | | | | | | | | | |
| RSAD2, IFI27, IFIT1, SIGLEC1, SLC1A2, IL1R2, | 0.888 | 0.879 | 0.897 | 0.934 | 0.887 | 0.981 | 0.943 | 0.874 | 1 |
| FAM20A | | | | | | | | | |
| RSAD2, IFI27, OAS1, IFIT1, ISG15, OLAH, | 0.888 | 0.879 | 0.897 | 0.921 | 0.861 | 0.981 | 0.933 | 0.865 | 1 |
| FAM20A | | | | | | | | | |
| IFI27, OAS1, IFIT1, IFI44L, OLAH, FAM20A, | 0.888 | 0.879 | 0.898 | 0.920 | 0.860 | 0.981 | 0.938 | 0.870 | 1 |
| RETN | | | | | | | | | |
| RSAD2, IFI27, OAS1, ISG15, HERC6, FAM20A, | 0.888 | 0.879 | 0.898 | 0.924 | 0.868 | 0.980 | 0.935 | 0.857 | 1 |
| RETN | | | | | | | | | |
| RSAD2, IFI27, IFIT1, IFI44L, SIGLEC1, | 0.888 | 0.879 | 0.897 | 0.919 | 0.858 | 0.979 | 0.945 | 0.877 | 1 |
| FAM20A, RETN | | | | | | | | | |
| RSAD2, IFI27, HERC6, SLC1A2, OLAH, | 0.888 | 0.879 | 0.897 | 0.931 | 0.879 | 0.982 | 0.918 | 0.834 | 1 |
| FAM20A, RETN | | | | | | | | | |
| IFI27, IFIT1, HERC6, IL1R2, OLAH, RETN, | 0.888 | 0.879 | 0.897 | 0.922 | 0.865 | 0.979 | 0.948 | 0.895 | 1 |
| MMP8 | | | | | | | | | |
| IFI27, IFI44L, SIGLEC1, SLC1A2, OLAH, | 0.888 | 0.879 | 0.897 | 0.926 | 0.869 | 0.982 | 0.946 | 0.879 | 1 |
| FAM20A, RETN | | | | | | | | | |
| RSAD2, IFI27, IFI44L, HERC6, OLAH, RETN, | 0.888 | 0.879 | 0.897 | 0.923 | 0.867 | 0.980 | 0.943 | 0.887 | 1.000 |
| MMP8 | | | | | | | | | |
| RSAD2, IFI27, ISG15, SLC1A2, IL1R2, OLAH, | 0.888 | 0.879 | 0.897 | 0.931 | 0.882 | 0.981 | 0.916 | 0.833 | 1.000 |
| FAM20A | | | | | | | | | |
| IFIT1, SIGLEC1, HERC6, IL1R2, FAM20A, | 0.888 | 0.879 | 0.897 | 0.922 | 0.866 | 0.978 | 0.961 | 0.901 | 1 |
| RETN, MMP8 | | | | | | | | | |
| IFI27, IFIT1, SIGLEC1, HERC6, SLC1A2, | 0.888 | 0.879 | 0.897 | 0.930 | 0.878 | 0.981 | 0.940 | 0.866 | 1 |
| FAM20A, RETN | | | | | | | | | |
| RSAD2, IFI27, IFI44L, ISG15, SLC1A2, OLAH, | 0.888 | 0.879 | 0.898 | 0.927 | 0.873 | 0.982 | 0.916 | 0.834 | 0.999 |
| FAM20A | | | | | | | | | |
| IFI27, IFIT1, SIGLEC1, ISG15, HERC6, IL1R2, | 0.888 | 0.880 | 0.896 | 0.926 | 0.876 | 0.976 | 0.941 | 0.888 | 0.995 |
| OLAH | | | | | | | | | |
| RSAD2, IFI27, OAS1, IFI44L, SIGLEC1, HERC6, | 0.888 | 0.880 | 0.896 | 0.927 | 0.876 | 0.977 | 0.946 | 0.894 | 0.998 |
| OLAH | | | | | | | | | |
| IFIT1, IFI44L, SIGLEC1, IL1R2, OLAH, | 0.888 | 0.879 | 0.897 | 0.922 | 0.866 | 0.978 | 0.965 | 0.913 | 1 |
| FAM20A, MMP8 | | | | | | | | | |
| RSAD2, IFI27, SIGLEC1, SLC1A2, OLAH, | 0.888 | 0.879 | 0.897 | 0.927 | 0.870 | 0.983 | 0.942 | 0.877 | 1 |
| FAM20A, RETN | | | | | | | | | |
| IFI27, IFIT1, ISG15, HERC6, OLAH, RETN, | 0.888 | 0.879 | 0.897 | 0.922 | 0.865 | 0.980 | 0.947 | 0.892 | 1 |
| MMP8 | | | | | | | | | |
| IFI27, IFIT1, IFI44L, SIGLEC1, HERC6, OLAH, | 0.888 | 0.880 | 0.897 | 0.924 | 0.871 | 0.977 | 0.947 | 0.897 | 0.997 |
| MMP8 | | | | | | | | | |
| IFI27, OAS1, IFI44L, IL1R2, FAM20A, RETN, | 0.888 | 0.879 | 0.897 | 0.929 | 0.876 | 0.981 | 0.935 | 0.856 | 1 |
| MMP8 | | | | | | | | | |
| IFI27, OAS1, IFIT1, ISG15, OLAH, FAM20A, | 0.888 | 0.879 | 0.897 | 0.920 | 0.860 | 0.980 | 0.935 | 0.867 | 1 |
| RETN | | | | | | | | | |
| IFI27, OAS1, ISG15, HERC6, IL1R2, OLAH, | 0.888 | 0.879 | 0.897 | 0.922 | 0.867 | 0.978 | 0.940 | 0.880 | 1 |
| RETN | | | | | | | | | |
| RSAD2, IFI27, IFIT1, SIGLEC1, HERC6, | 0.888 | 0.879 | 0.897 | 0.922 | 0.864 | 0.980 | 0.940 | 0.869 | 1 |
| FAM20A, MMP8 | | | | | | | | | |
| IFI27, ISG15, SLC1A2, IL1R2, OLAH, FAM20A, | 0.888 | 0.879 | 0.897 | 0.928 | 0.877 | 0.980 | 0.916 | 0.832 | 1 |
| RETN | | | | | | | | | |
| IFI27, IFIT1, IFI44L, SIGLEC1, HERC6, IL1R2, | 0.888 | 0.880 | 0.896 | 0.923 | 0.870 | 0.976 | 0.945 | 0.893 | 0.996 |
| RETN | | | | | | | | | |
| IFI27, IFIT1, ISG15, IL1R2, FAM20A, RETN, | 0.888 | 0.879 | 0.897 | 0.930 | 0.877 | 0.983 | 0.937 | 0.860 | 1 |
| MMP8 | | | | | | | | | |
| RSAD2, IFI27, IFIT1, IFI44L, HERC6, FAM20A, | 0.888 | 0.879 | 0.897 | 0.924 | 0.867 | 0.981 | 0.929 | 0.848 | 1 |
| RETN | | | | | | | | | |
| RSAD2, IFI27, OAS1, ISG15, HERC6, OLAH, | 0.888 | 0.879 | 0.897 | 0.923 | 0.867 | 0.979 | 0.943 | 0.885 | 1 |
| RETN | | | | | | | | | |
| RSAD2, IFI27, SIGLEC1, HERC6, IL1R2, RETN, | 0.888 | 0.879 | 0.897 | 0.922 | 0.869 | 0.976 | 0.961 | 0.920 | 1 |
| MMP8 | | | | | | | | | |
| RSAD2, IFI27, ISG15, HERC6, OLAH, RETN, | 0.888 | 0.879 | 0.897 | 0.924 | 0.867 | 0.980 | 0.945 | 0.889 | 1 |
| MMP8 | | | | | | | | | |
| IFI27, IFI44L, HERC6, SLC1A2, OLAH, | 0.888 | 0.878 | 0.898 | 0.927 | 0.873 | 0.981 | 0.926 | 0.848 | 1 |
| FAM20A, MMP8 | | | | | | | | | |
| IFI27, IFIT1, HERC6, SLC1A2, IL1R2, OLAH, | 0.888 | 0.879 | 0.897 | 0.930 | 0.880 | 0.981 | 0.927 | 0.848 | 1 |
| FAM20A | | | | | | | | | |
| IFI27, IFI44L, SLC1A2, OLAH, FAM20A, RETN, | 0.888 | 0.878 | 0.898 | 0.928 | 0.873 | 0.982 | 0.927 | 0.848 | 1 |
| MMP8 | | | | | | | | | |
| RSAD2, IFI27, OAS1, SIGLEC1, SLC1A2, IL1R2, | 0.888 | 0.879 | 0.897 | 0.932 | 0.884 | 0.980 | 0.942 | 0.871 | 1 |
| FAM20A | | | | | | | | | |
| IFI27, OAS1, SIGLEC1, SLC1A2, IL1R2, | 0.888 | 0.878 | 0.897 | 0.931 | 0.882 | 0.981 | 0.942 | 0.872 | 1 |
| FAM20A, RETN | | | | | | | | | |
| IFI27, OAS1, IFI44L, OLAH, FAM20A, RETN, | 0.888 | 0.878 | 0.898 | 0.923 | 0.863 | 0.983 | 0.942 | 0.877 | 1 |
| MMP8 | | | | | | | | | |
| IFI27, ISG15, HERC6, SLC1A2, OLAH, | 0.888 | 0.878 | 0.897 | 0.928 | 0.875 | 0.981 | 0.916 | 0.832 | 1 |
| FAM20A, RETN | | | | | | | | | |
| IFI27, OAS1, IFIT1, HERC6, IL1R2, OLAH, | 0.888 | 0.879 | 0.897 | 0.925 | 0.876 | 0.974 | 0.939 | 0.882 | 0.996 |
| MMP8 | | | | | | | | | |
| RSAD2, IFI27, IFIT1, SIGLEC1, ISG15, | 0.888 | 0.879 | 0.897 | 0.920 | 0.860 | 0.980 | 0.946 | 0.879 | 1 |
| FAM20A, RETN | | | | | | | | | |
| IFI27, IFI44L, SIGLEC1, IL1R2, OLAH, RETN, | 0.888 | 0.879 | 0.896 | 0.924 | 0.870 | 0.979 | 0.958 | 0.908 | 1 |
| MMP8 | | | | | | | | | |
| RSAD2, IFI27, IFI44L, OLAH, FAM20A, RETN, | 0.888 | 0.878 | 0.898 | 0.923 | 0.863 | 0.983 | 0.939 | 0.873 | 1 |
| MMP8 | | | | | | | | | |
| IFI27, OAS1, IFIT1, ISG15, IL1R2, FAM20A, | 0.888 | 0.879 | 0.897 | 0.926 | 0.874 | 0.978 | 0.933 | 0.854 | 1 |
| MMP8 | | | | | | | | | |
| RSAD2, IFI27, OAS1, IFIT1, SIGLEC1, HERC6, | 0.888 | 0.879 | 0.896 | 0.924 | 0.873 | 0.975 | 0.940 | 0.885 | 0.995 |
| OLAH | | | | | | | | | |
| IFI27, OAS1, ISG15, HERC6, FAM20A, RETN, | 0.888 | 0.878 | 0.898 | 0.925 | 0.868 | 0.982 | 0.935 | 0.857 | 1 |
| MMP8 | | | | | | | | | |
| RSAD2, OAS1, SIGLEC1, HERC6, IL1R2, | 0.888 | 0.879 | 0.897 | 0.920 | 0.865 | 0.976 | 0.961 | 0.902 | 1 |
| FAM20A, MMP8 | | | | | | | | | |
| IFI27, IFI44L, SIGLEC1, SLC1A2, IL1R2, | 0.888 | 0.879 | 0.897 | 0.930 | 0.879 | 0.980 | 0.947 | 0.877 | 1 |
| FAM20A, RETN | | | | | | | | | |
| IFI27, IFIT1, SIGLEC1, ISG15, HERC6, OLAH, | 0.888 | 0.879 | 0.896 | 0.922 | 0.869 | 0.975 | 0.941 | 0.887 | 0.995 |
| MMP8 | | | | | | | | | |
| IFI27, IFI44L, ISG15, SLC1A2, OLAH, FAM20A, | 0.888 | 0.878 | 0.897 | 0.928 | 0.874 | 0.982 | 0.917 | 0.836 | 0.999 |
| RETN | | | | | | | | | |
| IFI27, IFI44L, ISG15, HERC6, IL1R2, OLAH, | 0.888 | 0.879 | 0.896 | 0.927 | 0.877 | 0.976 | 0.934 | 0.874 | 0.993 |
| MMP8 | | | | | | | | | |
| RSAD2, IFI27, SIGLEC1, HERC6, IL1R2, OLAH, | 0.888 | 0.879 | 0.896 | 0.923 | 0.869 | 0.978 | 0.946 | 0.890 | 1 |
| RETN | | | | | | | | | |
| IFI27, IFIT1, SIGLEC1, HERC6, IL1R2, OLAH, | 0.888 | 0.879 | 0.896 | 0.926 | 0.876 | 0.975 | 0.943 | 0.892 | 0.995 |
| MMP8 | | | | | | | | | |
| IFI27, IFI44L, SIGLEC1, HERC6, IL1R2, RETN, | 0.888 | 0.879 | 0.896 | 0.924 | 0.871 | 0.977 | 0.952 | 0.906 | 0.998 |
| MMP8 | | | | | | | | | |
| IFI27, OAS1, IFI44L, HERC6, IL1R2, OLAH, | 0.888 | 0.879 | 0.896 | 0.928 | 0.880 | 0.976 | 0.935 | 0.876 | 0.994 |
| MMP8 | | | | | | | | | |
| IFI27, IFIT1, ISG15, SLC1A2, IL1R2, OLAH, | 0.888 | 0.879 | 0.897 | 0.929 | 0.878 | 0.980 | 0.920 | 0.839 | 1 |
| FAM20A | | | | | | | | | |
| RSAD2, IFI27, OAS1, ISG15, SLC1A2, OLAH, | 0.888 | 0.878 | 0.897 | 0.926 | 0.872 | 0.980 | 0.916 | 0.833 | 1.000 |
| FAM20A | | | | | | | | | |
| IFI27, SIGLEC1, ISG15, HERC6, IL1R2, OLAH, | 0.888 | 0.879 | 0.896 | 0.924 | 0.874 | 0.974 | 0.946 | 0.895 | 0.996 |
| MMP8 | | | | | | | | | |
| IFI27, SIGLEC1, ISG15, HERC6, OLAH, RETN, | 0.888 | 0.878 | 0.897 | 0.919 | 0.860 | 0.978 | 0.957 | 0.908 | 1 |
| MMP8 | | | | | | | | | |
| RSAD2, IFI27, OAS1, IFIT1, SIGLEC1, FAM20A, | 0.888 | 0.878 | 0.897 | 0.919 | 0.860 | 0.979 | 0.946 | 0.878 | 1 |
| RETN | | | | | | | | | |
| IFI27, OAS1, SIGLEC1, HERC6, SLC1A2, | 0.887 | 0.878 | 0.897 | 0.928 | 0.878 | 0.978 | 0.941 | 0.882 | 1 |
| OLAH, MMP8 | | | | | | | | | |
| IFI27, OAS1, IFI44L, SIGLEC1, ISG15, HERC6, | 0.887 | 0.879 | 0.896 | 0.926 | 0.875 | 0.977 | 0.943 | 0.890 | 0.997 |
| OLAH | | | | | | | | | |
| RSAD2, IFI27, IFIT1, IL1R2, OLAH, FAM20A, | 0.887 | 0.879 | 0.896 | 0.924 | 0.868 | 0.981 | 0.933 | 0.863 | 1 |
| RETN | | | | | | | | | |
| IFI27, OAS1, SIGLEC1, HERC6, SLC1A2, | 0.887 | 0.878 | 0.897 | 0.929 | 0.879 | 0.979 | 0.941 | 0.878 | 1 |
| OLAH, RETN | | | | | | | | | |
| IFI27, OAS1, ISG15, HERC6, IL1R2, OLAH, | 0.887 | 0.879 | 0.896 | 0.926 | 0.877 | 0.975 | 0.938 | 0.880 | 0.996 |
| MMP8 | | | | | | | | | |
| RSAD2, IFI27, SIGLEC1, ISG15, FAM20A, | 0.887 | 0.877 | 0.898 | 0.925 | 0.867 | 0.984 | 0.945 | 0.872 | 1 |
| RETN, MMP8 | | | | | | | | | |
| IFI27, IFIT1, IFI44L, SLC1A2, IL1R2, OLAH, | 0.887 | 0.879 | 0.896 | 0.928 | 0.874 | 0.982 | 0.921 | 0.838 | 1 |
| FAM20A | | | | | | | | | |
| IFI27, IFI44L, SIGLEC1, ISG15, FAM20A, | 0.887 | 0.877 | 0.897 | 0.922 | 0.862 | 0.981 | 0.948 | 0.875 | 1 |
| RETN, MMP8 | | | | | | | | | |
| RSAD2, IFI27, IFI44L, SIGLEC1, SLC1A2, | 0.887 | 0.878 | 0.897 | 0.930 | 0.880 | 0.980 | 0.945 | 0.875 | 1 |
| IL1R2, FAM20A | | | | | | | | | |
| RSAD2, IFI27, OAS1, SIGLEC1, HERC6, RETN, | 0.887 | 0.878 | 0.897 | 0.919 | 0.859 | 0.979 | 0.950 | 0.890 | 1 |
| MMP8 | | | | | | | | | |
| IFI27, OAS1, IFI44L, SIGLEC1, HERC6, RETN, | 0.887 | 0.879 | 0.896 | 0.919 | 0.862 | 0.977 | 0.945 | 0.884 | 1 |
| MMP8 | | | | | | | | | |
| RSAD2, IFI27, IFIT1, SIGLEC1, HERC6, IL1R2, | 0.887 | 0.879 | 0.896 | 0.926 | 0.877 | 0.975 | 0.940 | 0.887 | 0.994 |
| OLAH | | | | | | | | | |
| IFI27, IFIT1, IFI44L, ISG15, HERC6, OLAH, | 0.887 | 0.879 | 0.896 | 0.920 | 0.864 | 0.977 | 0.941 | 0.885 | 0.997 |
| RETN | | | | | | | | | |
| IFI27, ISG15, SLC1A2, OLAH, FAM20A, RETN, | 0.887 | 0.878 | 0.897 | 0.928 | 0.875 | 0.981 | 0.924 | 0.845 | 1 |
| MMP8 | | | | | | | | | |
| IFI27, OAS1, SIGLEC1, ISG15, HERC6, OLAH, | 0.887 | 0.879 | 0.896 | 0.922 | 0.869 | 0.976 | 0.938 | 0.881 | 0.995 |
| MMP8 | | | | | | | | | |
| RSAD2, IFI27, OAS1, IFI44L, HERC6, OLAH, | 0.887 | 0.879 | 0.896 | 0.922 | 0.865 | 0.978 | 0.941 | 0.882 | 1 |
| RETN | | | | | | | | | |
| RSAD2, IFI27, SIGLEC1, ISG15, HERC6, IL1R2, | 0.887 | 0.879 | 0.896 | 0.925 | 0.875 | 0.975 | 0.938 | 0.882 | 0.994 |
| OLAH | | | | | | | | | |
| IFI27, ISG15, HERC6, IL1R2, OLAH, RETN, | 0.887 | 0.878 | 0.896 | 0.922 | 0.865 | 0.979 | 0.948 | 0.894 | 1 |
| MMP8 | | | | | | | | | |
| RSAD2, IFI27, OAS1, IFIT1, HERC6, OLAH, | 0.887 | 0.879 | 0.896 | 0.923 | 0.871 | 0.975 | 0.935 | 0.876 | 0.994 |
| MMP8 | | | | | | | | | |
| IFI27, SIGLEC1, ISG15, HERC6, SLC1A2, | 0.887 | 0.878 | 0.897 | 0.932 | 0.884 | 0.980 | 0.947 | 0.877 | 1 |
| IL1R2, FAM20A | | | | | | | | | |
| IFI27, OAS1, IFI44L, ISG15, OLAH, FAM20A, | 0.887 | 0.878 | 0.897 | 0.920 | 0.860 | 0.981 | 0.934 | 0.864 | 1 |
| RETN | | | | | | | | | |
| IFI27, OAS1, IFIT1, SIGLEC1, ISG15, HERC6, | 0.887 | 0.879 | 0.896 | 0.923 | 0.871 | 0.975 | 0.938 | 0.881 | 0.995 |
| OLAH | | | | | | | | | |
| IFI27, IFIT1, IFI44L, SIGLEC1, IL1R2, OLAH, | 0.887 | 0.879 | 0.895 | 0.924 | 0.874 | 0.974 | 0.949 | 0.900 | 0.998 |
| MMP8 | | | | | | | | | |
| RSAD2, IFI27, SIGLEC1, SLC1A2, IL1R2, | 0.887 | 0.878 | 0.897 | 0.931 | 0.882 | 0.981 | 0.947 | 0.880 | 1 |
| FAM20A, RETN | | | | | | | | | |
| RSAD2, IFI27, IFIT1, IFI44L, HERC6, OLAH, | 0.887 | 0.879 | 0.896 | 0.923 | 0.868 | 0.977 | 0.936 | 0.875 | 0.996 |
| RETN | | | | | | | | | |
| IFI27, IFI44L, SIGLEC1, ISG15, HERC6, IL1R2, | 0.887 | 0.879 | 0.895 | 0.924 | 0.871 | 0.977 | 0.945 | 0.893 | 0.996 |
| RETN | | | | | | | | | |
| IFI27, IFI44L, SIGLEC1, ISG15, SLC1A2, OLAH, | 0.887 | 0.879 | 0.896 | 0.930 | 0.880 | 0.979 | 0.945 | 0.887 | 1 |
| MMP8 | | | | | | | | | |
| RSAD2, SIGLEC1, HERC6, IL1R2, OLAH, | 0.887 | 0.878 | 0.896 | 0.919 | 0.863 | 0.975 | 0.962 | 0.905 | 1 |
| FAM20A, MMP8 | | | | | | | | | |
| IFI27, OAS1, IFI44L, SIGLEC1, ISG15, FAM20A, | 0.887 | 0.878 | 0.897 | 0.920 | 0.861 | 0.980 | 0.946 | 0.877 | 1 |
| RETN | | | | | | | | | |
| IFI27, OAS1, SIGLEC1, ISG15, HERC6, SLC1A2, | 0.887 | 0.878 | 0.896 | 0.930 | 0.881 | 0.980 | 0.937 | 0.877 | 0.997 |
| OLAH | | | | | | | | | |
| IFI27, OAS1, IFI44L, ISG15, HERC6, OLAH, | 0.887 | 0.878 | 0.896 | 0.922 | 0.867 | 0.977 | 0.938 | 0.877 | 0.999 |
| RETN | | | | | | | | | |
| RSAD2, IFI27, IFI44L, HERC6, IL1R2, OLAH, | 0.887 | 0.879 | 0.896 | 0.926 | 0.877 | 0.976 | 0.936 | 0.878 | 0.993 |
| MMP8 | | | | | | | | | |
| RSAD2, IFI27, IFIT1, ISG15, IL1R2, FAM20A, | 0.887 | 0.878 | 0.896 | 0.925 | 0.873 | 0.978 | 0.933 | 0.854 | 1 |
| MMP8 | | | | | | | | | |
| RSAD2, IFI27, IFIT1, ISG15, SLC1A2, OLAH, | 0.887 | 0.878 | 0.896 | 0.925 | 0.870 | 0.980 | 0.921 | 0.841 | 1 |
| FAM20A | | | | | | | | | |
| IFI27, OAS1, IFIT1, HERC6, IL1R2, RETN, | 0.887 | 0.878 | 0.896 | 0.924 | 0.871 | 0.977 | 0.942 | 0.882 | 1 |
| MMP8 | | | | | | | | | |
| IFI27, OAS1, IFIT1, ISG15, SLC1A2, OLAH, | 0.887 | 0.878 | 0.896 | 0.925 | 0.869 | 0.980 | 0.924 | 0.848 | 0.999 |
| FAM20A | | | | | | | | | |
| IFI27, OAS1, IFIT1, IFI44L, HERC6, OLAH, | 0.887 | 0.878 | 0.896 | 0.926 | 0.874 | 0.977 | 0.935 | 0.876 | 0.994 |
| MMP8 | | | | | | | | | |
| RSAD2, IFI27, IFIT1, SIGLEC1, HERC6, OLAH, | 0.887 | 0.878 | 0.896 | 0.923 | 0.871 | 0.975 | 0.937 | 0.881 | 0.993 |
| MMP8 | | | | | | | | | |
| RSAD2, IFI27, OAS1, IFIT1, IFI44L, IL1R2, | 0.887 | 0.878 | 0.896 | 0.922 | 0.867 | 0.976 | 0.928 | 0.848 | 1 |
| FAM20A | | | | | | | | | |
| OAS1, IFIT1, IFI44L, SIGLEC1, IL1R2, | 0.887 | 0.878 | 0.896 | 0.920 | 0.863 | 0.978 | 0.965 | 0.912 | 1 |
| FAM20A, MMP8 | | | | | | | | | |
| IFI44L, SIGLEC1, ISG15, HERC6, IL1R2, | 0.887 | 0.878 | 0.896 | 0.918 | 0.860 | 0.975 | 0.959 | 0.897 | 1 |
| FAM20A, MMP8 | | | | | | | | | |
| IFI27, OAS1, IFIT1, IFI44L, IL1R2, FAM20A, | 0.887 | 0.878 | 0.896 | 0.923 | 0.869 | 0.977 | 0.930 | 0.852 | 1 |
| RETN | | | | | | | | | |
| IFI27, OAS1, IFI44L, ISG15, IL1R2, FAM20A, | 0.887 | 0.878 | 0.896 | 0.925 | 0.873 | 0.978 | 0.930 | 0.847 | 1 |
| MMP8 | | | | | | | | | |
| IFIT1, IFI44L, SIGLEC1, SLC1A2, OLAH, | 0.887 | 0.878 | 0.896 | 0.926 | 0.872 | 0.979 | 0.947 | 0.886 | 1 |
| FAM20A, MMP8 | | | | | | | | | |
| IFI27, OAS1, IFIT1, IFI44L, SIGLEC1, ISG15, | 0.887 | 0.878 | 0.896 | 0.915 | 0.850 | 0.979 | 0.943 | 0.871 | 1 |
| FAM20A | | | | | | | | | |
| IFI27, OAS1, IFIT1, IFI44L, ISG15, HERC6, | 0.887 | 0.878 | 0.896 | 0.916 | 0.856 | 0.976 | 0.935 | 0.869 | 1 |
| RETN | | | | | | | | | |
| RSAD2, IFI27, IFIT1, HERC6, IL1R2, OLAH, | 0.887 | 0.878 | 0.896 | 0.923 | 0.868 | 0.978 | 0.937 | 0.876 | 0.998 |
| RETN | | | | | | | | | |
| IFI27, IFI44L, HERC6, SLC1A2, IL1R2, OLAH, | 0.887 | 0.877 | 0.897 | 0.929 | 0.878 | 0.980 | 0.928 | 0.849 | 1 |
| FAM20A | | | | | | | | | |
| IFI27, SIGLEC1, SLC1A2, IL1R2, OLAH, | 0.887 | 0.878 | 0.896 | 0.927 | 0.872 | 0.981 | 0.945 | 0.877 | 1 |
| FAM20A, RETN | | | | | | | | | |
| IFI27, IFIT1, SIGLEC1, HERC6, IL1R2, RETN, | 0.887 | 0.878 | 0.895 | 0.921 | 0.867 | 0.976 | 0.957 | 0.914 | 0.999 |
| MMP8 | | | | | | | | | |
| RSAD2, IFI27, IFI44L, SIGLEC1, HERC6, IL1R2, | 0.887 | 0.879 | 0.895 | 0.924 | 0.871 | 0.977 | 0.945 | 0.893 | 0.996 |
| RETN | | | | | | | | | |
| IFI27, IFI44L, ISG15, HERC6, IL1R2, OLAH, | 0.887 | 0.878 | 0.895 | 0.923 | 0.869 | 0.977 | 0.941 | 0.885 | 0.997 |
| RETN | | | | | | | | | |
| RSAD2, IFI27, IFI44L, IL1R2, OLAH, FAM20A, | 0.887 | 0.877 | 0.897 | 0.924 | 0.866 | 0.981 | 0.929 | 0.856 | 1 |
| RETN | | | | | | | | | |
| OAS1, IFI44L, SIGLEC1, HERC6, IL1R2, | 0.887 | 0.878 | 0.896 | 0.919 | 0.863 | 0.976 | 0.960 | 0.900 | 1 |
| FAM20A, MMP8 | | | | | | | | | |
| RSAD2, IFI27, OAS1, IFI44L, HERC6, FAM20A, | 0.887 | 0.877 | 0.897 | 0.923 | 0.866 | 0.981 | 0.934 | 0.853 | 1 |
| RETN | | | | | | | | | |
| IFI27, OAS1, HERC6, SLC1A2, OLAH, RETN, | 0.887 | 0.878 | 0.896 | 0.928 | 0.878 | 0.978 | 0.938 | 0.874 | 1 |
| MMP8 | | | | | | | | | |
| RSAD2, IFI27, IFIT1, ISG15, OLAH, FAM20A, | 0.887 | 0.878 | 0.896 | 0.920 | 0.860 | 0.980 | 0.933 | 0.862 | 1 |
| RETN | | | | | | | | | |
| RSAD2, IFI27, OAS1, IL1R2, FAM20A, RETN, | 0.887 | 0.878 | 0.896 | 0.930 | 0.878 | 0.982 | 0.935 | 0.856 | 1 |
| MMP8 | | | | | | | | | |
| RSAD2, IFI27, IFIT1, ISG15, HERC6, FAM20A, | 0.887 | 0.878 | 0.896 | 0.924 | 0.869 | 0.980 | 0.933 | 0.852 | 1 |
| RETN | | | | | | | | | |
| RSAD2, IFI27, IFI44L, IL1R2, FAM20A, RETN, | 0.887 | 0.877 | 0.896 | 0.928 | 0.875 | 0.981 | 0.932 | 0.851 | 1 |
| MMP8 | | | | | | | | | |
| RSAD2, IFI27, IFIT1, IFI44L, SLC1A2, OLAH, | 0.887 | 0.878 | 0.896 | 0.926 | 0.870 | 0.982 | 0.923 | 0.844 | 1 |
| FAM20A | | | | | | | | | |
| RSAD2, IFI27, OAS1, IFIT1, HERC6, IL1R2, | 0.887 | 0.878 | 0.896 | 0.925 | 0.876 | 0.974 | 0.939 | 0.882 | 0.996 |
| OLAH | | | | | | | | | |
| RSAD2, IFI27, IFI44L, ISG15, IL1R2, FAM20A, | 0.887 | 0.877 | 0.896 | 0.926 | 0.874 | 0.978 | 0.929 | 0.846 | 1 |
| MMP8 | | | | | | | | | |
| IFI27, SIGLEC1, ISG15, SLC1A2, IL1R2, | 0.887 | 0.878 | 0.896 | 0.932 | 0.884 | 0.980 | 0.946 | 0.876 | 1 |
| FAM20A, RETN | | | | | | | | | |
| IFIT1, IFI44L, SIGLEC1, ISG15, HERC6, IL1R2, | 0.887 | 0.878 | 0.895 | 0.918 | 0.861 | 0.976 | 0.953 | 0.897 | 1 |
| FAM20A | | | | | | | | | |
| IFI27, OAS1, ISG15, SLC1A2, IL1R2, OLAH, | 0.887 | 0.878 | 0.896 | 0.930 | 0.879 | 0.981 | 0.916 | 0.834 | 0.999 |
| FAM20A | | | | | | | | | |
| RSAD2, IFI27, IFIT1, IFI44L, SIGLEC1, HERC6, | 0.887 | 0.878 | 0.895 | 0.924 | 0.872 | 0.977 | 0.947 | 0.897 | 0.997 |
| OLAH | | | | | | | | | |
| RSAD2, IFIT1, SIGLEC1, IL1R2, OLAH, | 0.887 | 0.878 | 0.895 | 0.921 | 0.864 | 0.977 | 0.966 | 0.915 | 1 |
| FAM20A, MMP8 | | | | | | | | | |
| IFI27, SIGLEC1, HERC6, IL1R2, OLAH, RETN, | 0.887 | 0.878 | 0.896 | 0.920 | 0.862 | 0.978 | 0.960 | 0.912 | 1 |
| MMP8 | | | | | | | | | |
| RSAD2, IFI27, IFI44L, ISG15, HERC6, FAM20A, | 0.887 | 0.877 | 0.896 | 0.926 | 0.871 | 0.981 | 0.937 | 0.860 | 1 |
| RETN | | | | | | | | | |
| IFI44L, SIGLEC1, HERC6, IL1R2, OLAH, | 0.887 | 0.878 | 0.896 | 0.918 | 0.861 | 0.976 | 0.961 | 0.903 | 1 |
| FAM20A, MMP8 | | | | | | | | | |
| RSAD2, OAS1, IFIT1, SIGLEC1, IL1R2, | 0.887 | 0.878 | 0.895 | 0.920 | 0.863 | 0.977 | 0.963 | 0.909 | 1 |
| FAM20A, MMP8 | | | | | | | | | |
| IFI27, IFIT1, IFI44L, ISG15, IL1R2, FAM20A, | 0.887 | 0.878 | 0.896 | 0.924 | 0.871 | 0.978 | 0.930 | 0.852 | 1 |
| RETN | | | | | | | | | |
| IFI27, OAS1, IFI44L, SIGLEC1, FAM20A, RETN, | 0.887 | 0.877 | 0.896 | 0.922 | 0.863 | 0.981 | 0.947 | 0.875 | 1 |
| MMP8 | | | | | | | | | |
| RSAD2, IFI27, OAS1, ISG15, OLAH, FAM20A, | 0.887 | 0.877 | 0.896 | 0.920 | 0.861 | 0.980 | 0.934 | 0.866 | 1 |
| RETN | | | | | | | | | |
| RSAD2, IFI27, IFIT1, ISG15, HERC6, OLAH, | 0.887 | 0.878 | 0.895 | 0.922 | 0.866 | 0.977 | 0.937 | 0.876 | 0.998 |
| RETN | | | | | | | | | |
| RSAD2, OAS1, SIGLEC1, IL1R2, OLAH, | 0.887 | 0.877 | 0.896 | 0.919 | 0.862 | 0.975 | 0.963 | 0.908 | 1 |
| FAM20A, MMP8 | | | | | | | | | |
| RSAD2, IFI27, SIGLEC1, ISG15, HERC6, OLAH, | 0.887 | 0.878 | 0.896 | 0.923 | 0.870 | 0.975 | 0.937 | 0.880 | 0.994 |
| MMP8 | | | | | | | | | |
| IFI27, IFI44L, SLC1A2, IL1R2, OLAH, FAM20A, | 0.887 | 0.877 | 0.896 | 0.927 | 0.873 | 0.982 | 0.921 | 0.838 | 1 |
| RETN | | | | | | | | | |
| IFI27, OAS1, IFI44L, SIGLEC1, HERC6, IL1R2, | 0.887 | 0.879 | 0.894 | 0.922 | 0.876 | 0.969 | 0.940 | 0.888 | 0.993 |
| MMP8 | | | | | | | | | |
| IFI27, OAS1, SIGLEC1, HERC6, SLC1A2, | 0.887 | 0.877 | 0.896 | 0.926 | 0.873 | 0.980 | 0.937 | 0.863 | 1 |
| FAM20A, RETN | | | | | | | | | |
| IFIT1, IFI44L, SIGLEC1, HERC6, OLAH, | 0.887 | 0.877 | 0.896 | 0.918 | 0.859 | 0.976 | 0.952 | 0.896 | 1 |
| FAM20A, MMP8 | | | | | | | | | |
| IFI27, IFI44L, SIGLEC1, ISG15, SLC1A2, IL1R2, | 0.887 | 0.878 | 0.895 | 0.928 | 0.878 | 0.978 | 0.943 | 0.888 | 0.999 |
| OLAH | | | | | | | | | |
| IFIT1, SIGLEC1, ISG15, IL1R2, OLAH, | 0.887 | 0.878 | 0.895 | 0.920 | 0.864 | 0.977 | 0.966 | 0.916 | 1 |
| FAM20A, MMP8 | | | | | | | | | |
| IFI27, OAS1, IFIT1, SIGLEC1, OLAH, RETN, | 0.887 | 0.877 | 0.896 | 0.920 | 0.861 | 0.978 | 0.962 | 0.911 | 1 |
| MMP8 | | | | | | | | | |
| RSAD2, IFI27, IFI44L, SIGLEC1, ISG15, HERC6, | 0.887 | 0.878 | 0.895 | 0.925 | 0.873 | 0.977 | 0.946 | 0.894 | 0.998 |
| OLAH | | | | | | | | | |
| IFI27, IFIT1, SIGLEC1, ISG15, SLC1A2, OLAH, | 0.887 | 0.878 | 0.895 | 0.930 | 0.881 | 0.980 | 0.938 | 0.878 | 0.998 |
| MMP8 | | | | | | | | | |
| IFI27, IFIT1, IFI44L, SIGLEC1, ISG15, IL1R2, | 0.887 | 0.878 | 0.895 | 0.924 | 0.874 | 0.975 | 0.945 | 0.893 | 0.996 |
| OLAH | | | | | | | | | |
| RSAD2, IFI27, IFIT1, IFI44L, OLAH, FAM20A, | 0.887 | 0.877 | 0.896 | 0.921 | 0.860 | 0.981 | 0.937 | 0.869 | 1 |
| RETN | | | | | | | | | |
| IFIT1, IFI44L, SIGLEC1, HERC6, IL1R2, OLAH, | 0.887 | 0.878 | 0.895 | 0.918 | 0.860 | 0.976 | 0.951 | 0.894 | 1 |
| FAM20A | | | | | | | | | |
| IFI27, IFIT1, IFI44L, SIGLEC1, ISG15, FAM20A, | 0.887 | 0.877 | 0.896 | 0.915 | 0.851 | 0.980 | 0.943 | 0.873 | 1 |
| MMP8 | | | | | | | | | |
| RSAD2, IFI27, OAS1, IFIT1, HERC6, FAM20A, | 0.887 | 0.877 | 0.896 | 0.923 | 0.865 | 0.981 | 0.934 | 0.853 | 1 |
| RETN | | | | | | | | | |
| IFI27, SIGLEC1, ISG15, HERC6, FAM20A, | 0.887 | 0.876 | 0.897 | 0.919 | 0.857 | 0.982 | 0.953 | 0.881 | 1 |
| RETN, MMP8 | | | | | | | | | |
| RSAD2, IFI27, ISG15, HERC6, IL1R2, OLAH, | 0.887 | 0.878 | 0.895 | 0.923 | 0.868 | 0.978 | 0.938 | 0.877 | 0.999 |
| RETN | | | | | | | | | |
| IFI27, OAS1, IFI44L, SIGLEC1, IL1R2, OLAH, | 0.886 | 0.878 | 0.895 | 0.923 | 0.872 | 0.974 | 0.949 | 0.900 | 0.997 |
| MMP8 | | | | | | | | | |
| RSAD2, IFI27, OAS1, ISG15, HERC6, OLAH, | 0.886 | 0.878 | 0.895 | 0.923 | 0.871 | 0.975 | 0.937 | 0.879 | 0.995 |
| MMP8 | | | | | | | | | |
| RSAD2, IFI27, OAS1, SLC1A2, IL1R2, OLAH, | 0.886 | 0.878 | 0.895 | 0.928 | 0.875 | 0.981 | 0.918 | 0.834 | 1 |
| FAM20A | | | | | | | | | |
| RSAD2, IFI27, IFI44L, SIGLEC1, IL1R2, OLAH, | 0.886 | 0.878 | 0.895 | 0.924 | 0.874 | 0.974 | 0.950 | 0.902 | 0.998 |
| MMP8 | | | | | | | | | |
| RSAD2, IFI27, OAS1, IFI44L, HERC6, IL1R2, | 0.886 | 0.878 | 0.895 | 0.928 | 0.879 | 0.976 | 0.936 | 0.877 | 0.995 |
| OLAH | | | | | | | | | |
| IFI27, HERC6, SLC1A2, OLAH, FAM20A, | 0.886 | 0.877 | 0.896 | 0.929 | 0.876 | 0.982 | 0.925 | 0.844 | 1 |
| RETN, MMP8 | | | | | | | | | |
| IFI27, OAS1, IFI44L, HERC6, IL1R2, OLAH, | 0.886 | 0.878 | 0.895 | 0.922 | 0.867 | 0.977 | 0.935 | 0.872 | 0.998 |
| RETN | | | | | | | | | |
| IFI27, OAS1, IFIT1, SIGLEC1, HERC6, IL1R2, | 0.886 | 0.879 | 0.894 | 0.922 | 0.875 | 0.968 | 0.948 | 0.900 | 0.996 |
| MMP8 | | | | | | | | | |
| IFI27, SIGLEC1, ISG15, SLC1A2, IL1R2, OLAH, | 0.886 | 0.878 | 0.895 | 0.930 | 0.881 | 0.978 | 0.941 | 0.885 | 0.997 |
| MMP8 | | | | | | | | | |
| RSAD2, IFI27, OAS1, IFI44L, HERC6, OLAH, | 0.886 | 0.878 | 0.895 | 0.925 | 0.874 | 0.977 | 0.934 | 0.874 | 0.994 |
| MMP8 | | | | | | | | | |
| IFI27, OAS1, IFIT1, SLC1A2, IL1R2, OLAH, | 0.886 | 0.877 | 0.895 | 0.926 | 0.872 | 0.980 | 0.923 | 0.842 | 1 |
| FAM20A | | | | | | | | | |
| RSAD2, IFI27, OAS1, IFIT1, HERC6, IL1R2, | 0.886 | 0.878 | 0.895 | 0.922 | 0.868 | 0.976 | 0.945 | 0.885 | 1 |
| RETN | | | | | | | | | |
| RSAD2, IFI27, SIGLEC1, ISG15, HERC6, OLAH, | 0.886 | 0.878 | 0.895 | 0.921 | 0.866 | 0.977 | 0.942 | 0.886 | 0.999 |
| RETN | | | | | | | | | |
| RSAD2, IFI27, IFI44L, SIGLEC1, FAM20A, | 0.886 | 0.876 | 0.896 | 0.924 | 0.866 | 0.983 | 0.948 | 0.876 | 1 |
| RETN, MMP8 | | | | | | | | | |
| RSAD2, IFI27, IFI44L, ISG15, OLAH, FAM20A, | 0.886 | 0.877 | 0.896 | 0.921 | 0.86 | 0.981 | 0.934 | 0.864 | 1 |
| RETN | | | | | | | | | |
| RSAD2, IFI27, IFIT1, HERC6, IL1R2, OLAH, | 0.886 | 0.878 | 0.895 | 0.925 | 0.876 | 0.975 | 0.940 | 0.885 | 0.995 |
| MMP8 | | | | | | | | | |
| IFI27, OAS1, SIGLEC1, IL1R2, OLAH, RETN, | 0.886 | 0.877 | 0.895 | 0.922 | 0.867 | 0.978 | 0.961 | 0.910 | 1 |
| MMP8 | | | | | | | | | |
| IFI27, HERC6, SLC1A2, IL1R2, OLAH, | 0.886 | 0.877 | 0.895 | 0.929 | 0.876 | 0.981 | 0.927 | 0.848 | 1 |
| FAM20A, RETN | | | | | | | | | |
| RSAD2, IFI27, OAS1, ISG15, HERC6, IL1R2, | 0.886 | 0.878 | 0.895 | 0.925 | 0.876 | 0.975 | 0.938 | 0.880 | 0.996 |
| OLAH | | | | | | | | | |
| IFI27, OAS1, SIGLEC1, HERC6, SLC1A2, IL1R2, | 0.886 | 0.877 | 0.895 | 0.928 | 0.879 | 0.977 | 0.941 | 0.882 | 1 |
| OLAH | | | | | | | | | |
| RSAD2, IFI27, OAS1, IFIT1, IFI44L, SIGLEC1, | 0.886 | 0.877 | 0.895 | 0.915 | 0.851 | 0.980 | 0.946 | 0.877 | 1 |
| FAM20A | | | | | | | | | |
| IFI27, OAS1, IFI44L, ISG15, HERC6, OLAH, | 0.886 | 0.877 | 0.895 | 0.924 | 0.872 | 0.976 | 0.932 | 0.870 | 0.993 |
| MMP8 | | | | | | | | | |
| IFI27, OAS1, IFIT1, ISG15, HERC6, OLAH, | 0.886 | 0.877 | 0.895 | 0.923 | 0.871 | 0.975 | 0.937 | 0.879 | 0.995 |
| MMP8 | | | | | | | | | |
| OAS1, IFIT1, IFI44L, SIGLEC1, HERC6, IL1R2, | 0.886 | 0.878 | 0.895 | 0.918 | 0.861 | 0.976 | 0.950 | 0.893 | 1 |
| FAM20A | | | | | | | | | |
| RSAD2, IFI27, IFI44L, SLC1A2, IL1R2, OLAH, | 0.886 | 0.877 | 0.896 | 0.927 | 0.873 | 0.982 | 0.922 | 0.841 | 1 |
| FAM20A | | | | | | | | | |
| IFI27, OAS1, ISG15, IL1R2, FAM20A, RETN, | 0.886 | 0.877 | 0.895 | 0.929 | 0.877 | 0.980 | 0.934 | 0.855 | 1 |
| MMP8 | | | | | | | | | |
| RSAD2, IFI27, OAS1, IFI44L, OLAH, FAM20A, | 0.886 | 0.877 | 0.896 | 0.921 | 0.861 | 0.982 | 0.936 | 0.867 | 1 |
| RETN | | | | | | | | | |
| IFI27, IFI44L, SIGLEC1, ISG15, OLAH, RETN, | 0.886 | 0.877 | 0.895 | 0.921 | 0.865 | 0.978 | 0.959 | 0.908 | 1 |
| MMP8 | | | | | | | | | |
| RSAD2, IFI27, OAS1, HERC6, IL1R2, RETN, | 0.886 | 0.878 | 0.895 | 0.920 | 0.865 | 0.975 | 0.940 | 0.878 | 1 |
| MMP8 | | | | | | | | | |
| RSAD2, IFI27, OAS1, IFIT1, OLAH, FAM20A, | 0.886 | 0.877 | 0.895 | 0.919 | 0.858 | 0.980 | 0.937 | 0.869 | 1 |
| RETN | | | | | | | | | |
| RSAD2, IFI27, OAS1, SIGLEC1, ISG15, | 0.886 | 0.876 | 0.896 | 0.919 | 0.858 | 0.979 | 0.948 | 0.881 | 1 |
| FAM20A, RETN | | | | | | | | | |
| IFI27, IFIT1, SIGLEC1, IL1R2, OLAH, RETN, | 0.886 | 0.877 | 0.895 | 0.921 | 0.864 | 0.978 | 0.961 | 0.913 | 1 |
| MMP8 | | | | | | | | | |
| IFI27, OAS1, IFIT1, SIGLEC1, IL1R2, OLAH, | 0.886 | 0.878 | 0.894 | 0.922 | 0.871 | 0.973 | 0.947 | 0.896 | 0.998 |
| MMP8 | | | | | | | | | |
| RSAD2, IFI27, OAS1, SIGLEC1, ISG15, HERC6, | 0.886 | 0.877 | 0.895 | 0.923 | 0.871 | 0.976 | 0.938 | 0.881 | 0.995 |
| OLAH | | | | | | | | | |
| IFI27, IFIT1, SIGLEC1, ISG15, SLC1A2, IL1R2, | 0.886 | 0.878 | 0.895 | 0.929 | 0.879 | 0.978 | 0.939 | 0.880 | 0.998 |
| OLAH | | | | | | | | | |
| IFI27, IFIT1, SIGLEC1, ISG15, OLAH, RETN, | 0.886 | 0.877 | 0.895 | 0.920 | 0.861 | 0.979 | 0.958 | 0.905 | 1 |
| MMP8 | | | | | | | | | |
| RSAD2, IFI27, ISG15, HERC6, IL1R2, OLAH, | 0.886 | 0.878 | 0.895 | 0.924 | 0.874 | 0.975 | 0.939 | 0.884 | 0.995 |
| MMP8 | | | | | | | | | |
| IFI27, OAS1, IFIT1, IFI44L, ISG15, IL1R2, | 0.886 | 0.877 | 0.895 | 0.923 | 0.869 | 0.977 | 0.928 | 0.848 | 1 |
| FAM20A | | | | | | | | | |
| RSAD2, IFI27, IFIT1, SLC1A2, IL1R2, OLAH, | 0.886 | 0.877 | 0.895 | 0.926 | 0.872 | 0.980 | 0.922 | 0.841 | 1 |
| FAM20A | | | | | | | | | |
| IFI27, OAS1, IFIT1, SIGLEC1, HERC6, SLC1A2, | 0.886 | 0.877 | 0.895 | 0.927 | 0.877 | 0.978 | 0.941 | 0.883 | 1.000 |
| OLAH | | | | | | | | | |
| RSAD2, IFI27, IFI44L, HERC6, IL1R2, OLAH, | 0.886 | 0.877 | 0.895 | 0.924 | 0.870 | 0.978 | 0.936 | 0.876 | 0.996 |
| RETN | | | | | | | | | |
| IFI27, IFIT1, IFI44L, ISG15, HERC6, OLAH, | 0.886 | 0.877 | 0.895 | 0.922 | 0.868 | 0.976 | 0.935 | 0.876 | 0.993 |
| MMP8 | | | | | | | | | |
| IFI27, IFIT1, ISG15, SLC1A2, OLAH, FAM20A, | 0.886 | 0.877 | 0.895 | 0.925 | 0.869 | 0.981 | 0.922 | 0.844 | 1.000 |
| RETN | | | | | | | | | |
| IFIT1, IFI44L, SIGLEC1, ISG15, IL1R2, | 0.886 | 0.877 | 0.895 | 0.920 | 0.864 | 0.977 | 0.967 | 0.916 | 1 |
| FAM20A, MMP8 | | | | | | | | | |
| RSAD2, IFI27, SLC1A2, OLAH, FAM20A, | 0.886 | 0.876 | 0.896 | 0.927 | 0.872 | 0.982 | 0.924 | 0.844 | 1 |
| RETN, MMP8 | | | | | | | | | |
| RSAD2, IFI27, OAS1, IFIT1, SLC1A2, OLAH, | 0.886 | 0.877 | 0.895 | 0.924 | 0.867 | 0.981 | 0.922 | 0.841 | 1 |
| FAM20A | | | | | | | | | |
| IFI27, IFIT1, SLC1A2, OLAH, FAM20A, RETN, | 0.886 | 0.876 | 0.896 | 0.926 | 0.870 | 0.981 | 0.929 | 0.854 | 1 |
| MMP8 | | | | | | | | | |
| RSAD2, IFI27, OAS1, IFIT1, ISG15, HERC6, | 0.886 | 0.877 | 0.895 | 0.919 | 0.859 | 0.979 | 0.934 | 0.865 | 1 |
| RETN | | | | | | | | | |
| IFI27, IFIT1, SIGLEC1, ISG15, IL1R2, OLAH, | 0.886 | 0.878 | 0.894 | 0.923 | 0.872 | 0.974 | 0.943 | 0.892 | 0.995 |
| MMP8 | | | | | | | | | |
| IFI27, IFIT1, HERC6, SLC1A2, OLAH, FAM20A, | 0.886 | 0.876 | 0.896 | 0.928 | 0.875 | 0.982 | 0.922 | 0.839 | 1 |
| RETN | | | | | | | | | |
| IFI27, IFI44L, SIGLEC1, ISG15, IL1R2, OLAH, | 0.886 | 0.878 | 0.894 | 0.921 | 0.867 | 0.975 | 0.952 | 0.900 | 1 |
| RETN | | | | | | | | | |
| IFIT1, SIGLEC1, ISG15, HERC6, IL1R2, OLAH, | 0.886 | 0.877 | 0.895 | 0.919 | 0.862 | 0.977 | 0.952 | 0.895 | 1 |
| FAM20A | | | | | | | | | |
| IFI27, OAS1, IFIT1, IFI44L, SIGLEC1, HERC6, | 0.886 | 0.878 | 0.894 | 0.923 | 0.876 | 0.970 | 0.939 | 0.886 | 0.992 |
| IL1R2 | | | | | | | | | |
| IFI27, SIGLEC1, ISG15, IL1R2, OLAH, RETN, | 0.886 | 0.877 | 0.895 | 0.921 | 0.865 | 0.978 | 0.960 | 0.911 | 1 |
| MMP8 | | | | | | | | | |
| IFI27, IFI44L, ISG15, SLC1A2, IL1R2, FAM20A, | 0.886 | 0.877 | 0.895 | 0.930 | 0.883 | 0.976 | 0.926 | 0.839 | 1 |
| MMP8 | | | | | | | | | |
| RSAD2, IFI27, OAS1, SIGLEC1, ISG15, HERC6, | 0.886 | 0.876 | 0.895 | 0.918 | 0.858 | 0.977 | 0.940 | 0.868 | 1 |
| FAM20A | | | | | | | | | |
| RSAD2, IFI27, OAS1, SIGLEC1, HERC6, | 0.886 | 0.876 | 0.896 | 0.916 | 0.856 | 0.976 | 0.941 | 0.871 | 1 |
| FAM20A, MMP8 | | | | | | | | | |
| RSAD2, IFI27, IFIT1, SIGLEC1, HERC6, IL1R2, | 0.886 | 0.878 | 0.894 | 0.923 | 0.871 | 0.975 | 0.949 | 0.901 | 0.997 |
| RETN | | | | | | | | | |
| RSAD2, IFI27, OAS1, IFI44L, SLC1A2, OLAH, | 0.886 | 0.876 | 0.895 | 0.929 | 0.874 | 0.984 | 0.923 | 0.843 | 1 |
| FAM20A | | | | | | | | | |
| OAS1, IFIT1, SIGLEC1, ISG15, IL1R2, FAM20A, | 0.886 | 0.877 | 0.895 | 0.918 | 0.860 | 0.976 | 0.965 | 0.910 | 1 |
| MMP8 | | | | | | | | | |
| IFI27, OAS1, IFI44L, SIGLEC1, ISG15, IL1R2, | 0.886 | 0.878 | 0.894 | 0.923 | 0.872 | 0.974 | 0.942 | 0.889 | 0.996 |
| OLAH | | | | | | | | | |
| IFI27, OAS1, SIGLEC1, ISG15, SLC1A2, OLAH, | 0.886 | 0.877 | 0.894 | 0.929 | 0.878 | 0.979 | 0.941 | 0.883 | 0.999 |
| MMP8 | | | | | | | | | |
| IFI27, IFIT1, IFI44L, HERC6, FAM20A, RETN, | 0.886 | 0.876 | 0.896 | 0.921 | 0.862 | 0.980 | 0.932 | 0.849 | 1 |
| MMP8 | | | | | | | | | |
| OAS1, IFIT1, SIGLEC1, HERC6, IL1R2, OLAH, | 0.886 | 0.877 | 0.895 | 0.919 | 0.861 | 0.976 | 0.952 | 0.895 | 1 |
| FAM20A | | | | | | | | | |
| IFI27, IFIT1, IFI44L, SIGLEC1, OLAH, RETN, | 0.886 | 0.876 | 0.895 | 0.921 | 0.863 | 0.979 | 0.959 | 0.908 | 1 |
| MMP8 | | | | | | | | | |
| IFI27, OAS1, IFIT1, ISG15, HERC6, IL1R2, | 0.886 | 0.877 | 0.894 | 0.924 | 0.872 | 0.976 | 0.941 | 0.881 | 1 |
| RETN | | | | | | | | | |
| RSAD2, IFI27, ISG15, SLC1A2, OLAH, | 0.886 | 0.876 | 0.895 | 0.927 | 0.873 | 0.981 | 0.916 | 0.833 | 1.000 |
| FAM20A, RETN | | | | | | | | | |
| IFI27, IFIT1, IFI44L, SLC1A2, OLAH, FAM20A, | 0.886 | 0.876 | 0.895 | 0.925 | 0.868 | 0.982 | 0.922 | 0.841 | 1 |
| RETN | | | | | | | | | |
| RSAD2, IFI27, OAS1, SIGLEC1, HERC6, | 0.886 | 0.876 | 0.895 | 0.928 | 0.878 | 0.978 | 0.941 | 0.882 | 1 |
| SLC1A2, OLAH | | | | | | | | | |
| RSAD2, IFI27, OAS1, IFI44L, SIGLEC1, HERC6, | 0.886 | 0.878 | 0.894 | 0.925 | 0.878 | 0.972 | 0.946 | 0.896 | 0.995 |
| IL1R2 | | | | | | | | | |
| IFI27, IFI44L, SIGLEC1, ISG15, SLC1A2, OLAH, | 0.886 | 0.877 | 0.894 | 0.928 | 0.877 | 0.979 | 0.947 | 0.889 | 1 |
| RETN | | | | | | | | | |
| RSAD2, IFI44L, SIGLEC1, HERC6, IL1R2, | 0.886 | 0.877 | 0.894 | 0.919 | 0.863 | 0.975 | 0.960 | 0.900 | 1 |
| FAM20A, MMP8 | | | | | | | | | |
| IFI27, IFIT1, ISG15, HERC6, IL1R2, OLAH, | 0.886 | 0.877 | 0.894 | 0.921 | 0.865 | 0.977 | 0.941 | 0.884 | 0.999 |
| RETN | | | | | | | | | |
| RSAD2, IFI27, ISG15, HERC6, FAM20A, RETN, | 0.886 | 0.876 | 0.895 | 0.927 | 0.871 | 0.982 | 0.934 | 0.854 | 1 |
| MMP8 | | | | | | | | | |
| IFI27, SIGLEC1, HERC6, SLC1A2, OLAH, | 0.886 | 0.876 | 0.895 | 0.926 | 0.869 | 0.983 | 0.943 | 0.876 | 1 |
| FAM20A, RETN | | | | | | | | | |
| IFI27, OAS1, IFI44L, HERC6, FAM20A, RETN, | 0.886 | 0.876 | 0.896 | 0.924 | 0.865 | 0.983 | 0.935 | 0.855 | 1 |
| MMP8 | | | | | | | | | |
| IFI27, OAS1, IFI44L, ISG15, IL1R2, FAM20A, | 0.886 | 0.877 | 0.895 | 0.925 | 0.872 | 0.978 | 0.929 | 0.850 | 1 |
| RETN | | | | | | | | | |
| IFI27, IFIT1, SIGLEC1, ISG15, HERC6, SLC1A2, | 0.886 | 0.877 | 0.895 | 0.930 | 0.880 | 0.980 | 0.939 | 0.881 | 0.997 |
| OLAH | | | | | | | | | |
| IFI27, OAS1, IFI44L, ISG15, HERC6, IL1R2, | 0.886 | 0.877 | 0.894 | 0.927 | 0.878 | 0.976 | 0.934 | 0.874 | 0.993 |
| OLAH | | | | | | | | | |
| IFI27, OAS1, IFI44L, SIGLEC1, OLAH, RETN, | 0.886 | 0.877 | 0.894 | 0.920 | 0.863 | 0.978 | 0.959 | 0.908 | 1 |
| MMP8 | | | | | | | | | |
| RSAD2, IFI27, OAS1, ISG15, IL1R2, FAM20A, | 0.886 | 0.877 | 0.895 | 0.927 | 0.875 | 0.978 | 0.928 | 0.843 | 1 |
| MMP8 | | | | | | | | | |
| IFI27, IFI44L, SLC1A2, IL1R2, FAM20A, RETN, | 0.886 | 0.877 | 0.895 | 0.930 | 0.880 | 0.980 | 0.924 | 0.836 | 1 |
| MMP8 | | | | | | | | | |
| IFI27, OAS1, IFIT1, ISG15, IL1R2, FAM20A, | 0.886 | 0.877 | 0.895 | 0.926 | 0.873 | 0.978 | 0.930 | 0.852 | 1 |
| RETN | | | | | | | | | |
| IFIT1, SIGLEC1, HERC6, OLAH, FAM20A, | 0.886 | 0.876 | 0.895 | 0.922 | 0.864 | 0.980 | 0.954 | 0.898 | 1 |
| RETN, MMP8 | | | | | | | | | |
| IFI44L, SIGLEC1, ISG15, IL1R2, OLAH, | 0.886 | 0.877 | 0.894 | 0.917 | 0.860 | 0.974 | 0.963 | 0.908 | 1 |
| FAM20A, MMP8 | | | | | | | | | |
| IFI27, OAS1, IFI44L, SLC1A2, IL1R2, OLAH, | 0.886 | 0.876 | 0.895 | 0.929 | 0.876 | 0.983 | 0.918 | 0.836 | 1 |
| FAM20A | | | | | | | | | |
| RSAD2, IFI27, OAS1, IFI44L, ISG15, IL1R2, | 0.886 | 0.877 | 0.894 | 0.923 | 0.869 | 0.977 | 0.930 | 0.851 | 1 |
| FAM20A | | | | | | | | | |
| IFI27, OAS1, IFIT1, SIGLEC1, HERC6, SLC1A2, | 0.886 | 0.877 | 0.894 | 0.926 | 0.874 | 0.978 | 0.940 | 0.880 | 1 |
| RETN | | | | | | | | | |
| RSAD2, IFI27, IFIT1, IFI44L, ISG15, IL1R2, | 0.885 | 0.877 | 0.894 | 0.922 | 0.868 | 0.977 | 0.928 | 0.848 | 1 |
| FAM20A | | | | | | | | | |
| IFI27, OAS1, ISG15, SLC1A2, OLAH, FAM20A, | 0.885 | 0.876 | 0.895 | 0.927 | 0.873 | 0.981 | 0.916 | 0.834 | 0.999 |
| RETN | | | | | | | | | |
| RSAD2, IFI27, IFI44L, ISG15, HERC6, OLAH, | 0.885 | 0.877 | 0.894 | 0.923 | 0.868 | 0.977 | 0.935 | 0.874 | 0.996 |
| RETN | | | | | | | | | |
| OAS1, SIGLEC1, HERC6, IL1R2, OLAH, | 0.885 | 0.876 | 0.895 | 0.919 | 0.863 | 0.975 | 0.963 | 0.908 | 1 |
| FAM20A, MMP8 | | | | | | | | | |
| IFI27, IFIT1, IFI44L, SIGLEC1, ISG15, HERC6, | 0.885 | 0.877 | 0.894 | 0.912 | 0.851 | 0.973 | 0.940 | 0.886 | 0.995 |
| RETN | | | | | | | | | |
| RSAD2, IFI27, OAS1, SIGLEC1, HERC6, IL1R2, | 0.885 | 0.877 | 0.894 | 0.922 | 0.874 | 0.969 | 0.951 | 0.905 | 0.997 |
| MMP8 | | | | | | | | | |
| IFI27, OAS1, IFIT1, IFI44L, HERC6, IL1R2, | 0.885 | 0.877 | 0.894 | 0.926 | 0.877 | 0.975 | 0.939 | 0.883 | 0.995 |
| OLAH | | | | | | | | | |
| RSAD2, IFIT1, SIGLEC1, ISG15, IL1R2, | 0.885 | 0.877 | 0.894 | 0.920 | 0.862 | 0.977 | 0.966 | 0.913 | 1 |
| FAM20A, MMP8 | | | | | | | | | |
| IFI27, OAS1, IFIT1, IFI44L, SIGLEC1, IL1R2, | 0.885 | 0.877 | 0.893 | 0.922 | 0.871 | 0.973 | 0.948 | 0.897 | 0.998 |
| OLAH | | | | | | | | | |
| IFI27, SIGLEC1, ISG15, HERC6, SLC1A2, | 0.885 | 0.876 | 0.895 | 0.930 | 0.880 | 0.980 | 0.939 | 0.880 | 0.998 |
| OLAH, MMP8 | | | | | | | | | |
| IFI27, OAS1, IFIT1, SIGLEC1, IL1R2, OLAH, | 0.885 | 0.877 | 0.894 | 0.919 | 0.864 | 0.975 | 0.948 | 0.892 | 1 |
| RETN | | | | | | | | | |
| RSAD2, IFI27, OAS1, HERC6, FAM20A, RETN, | 0.885 | 0.875 | 0.895 | 0.923 | 0.865 | 0.981 | 0.934 | 0.853 | 1 |
| MMP8 | | | | | | | | | |
| RSAD2, IFI27, SIGLEC1, IL1R2, OLAH, RETN, | 0.885 | 0.876 | 0.894 | 0.922 | 0.867 | 0.978 | 0.959 | 0.908 | 1 |
| MMP8 | | | | | | | | | |
| IFI27, IFIT1, SIGLEC1, SLC1A2, FAM20A, | 0.885 | 0.876 | 0.895 | 0.925 | 0.870 | 0.981 | 0.946 | 0.869 | 1 |
| RETN, MMP8 | | | | | | | | | |
| SIGLEC1, ISG15, HERC6, IL1R2, OLAH, | 0.885 | 0.876 | 0.895 | 0.916 | 0.858 | 0.974 | 0.964 | 0.909 | 1 |
| FAM20A, MMP8 | | | | | | | | | |
| RSAD2, IFI27, IFI44L, SIGLEC1, ISG15, IL1R2, | 0.885 | 0.877 | 0.893 | 0.925 | 0.874 | 0.975 | 0.943 | 0.891 | 0.996 |
| OLAH | | | | | | | | | |
| IFI27, OAS1, ISG15, HERC6, IL1R2, RETN, | 0.885 | 0.876 | 0.894 | 0.924 | 0.871 | 0.977 | 0.937 | 0.874 | 1.000 |
| MMP8 | | | | | | | | | |
| RSAD2, IFI27, IFIT1, SIGLEC1, ISG15, HERC6, | 0.885 | 0.877 | 0.894 | 0.924 | 0.873 | 0.976 | 0.939 | 0.884 | 0.994 |
| OLAH | | | | | | | | | |
| IFI27, IFIT1, IFI44L, HERC6, IL1R2, OLAH, | 0.885 | 0.877 | 0.894 | 0.922 | 0.868 | 0.977 | 0.943 | 0.888 | 0.999 |
| RETN | | | | | | | | | |
| RSAD2, IFI27, OAS1, IFI44L, SIGLEC1, HERC6, | 0.885 | 0.876 | 0.894 | 0.917 | 0.857 | 0.977 | 0.942 | 0.870 | 1 |
| FAM20A | | | | | | | | | |
| IFI27, OAS1, SIGLEC1, SLC1A2, IL1R2, OLAH, | 0.885 | 0.877 | 0.894 | 0.927 | 0.878 | 0.976 | 0.947 | 0.892 | 1 |
| MMP8 | | | | | | | | | |
| IFI27, OAS1, HERC6, SLC1A2, IL1R2, OLAH, | 0.885 | 0.877 | 0.894 | 0.927 | 0.878 | 0.976 | 0.929 | 0.862 | 0.997 |
| MMP8 | | | | | | | | | |
| RSAD2, IFI27, OAS1, ISG15, HERC6, IL1R2, | 0.885 | 0.877 | 0.894 | 0.922 | 0.868 | 0.976 | 0.942 | 0.881 | 1 |
| RETN | | | | | | | | | |
| RSAD2, IFI27, SLC1A2, IL1R2, OLAH, | 0.885 | 0.876 | 0.894 | 0.927 | 0.873 | 0.981 | 0.920 | 0.836 | 1 |
| FAM20A, RETN | | | | | | | | | |
| RSAD2, IFI27, OAS1, IFIT1, IL1R2, FAM20A, | 0.885 | 0.876 | 0.894 | 0.924 | 0.870 | 0.977 | 0.929 | 0.850 | 1 |
| RETN | | | | | | | | | |
| RSAD2, SIGLEC1, ISG15, HERC6, IL1R2, | 0.885 | 0.876 | 0.894 | 0.919 | 0.862 | 0.975 | 0.961 | 0.902 | 1 |
| FAM20A, MMP8 | | | | | | | | | |
| RSAD2, IFI27, OAS1, IFI44L, ISG15, HERC6, | 0.885 | 0.876 | 0.894 | 0.925 | 0.873 | 0.976 | 0.937 | 0.879 | 0.995 |
| OLAH | | | | | | | | | |
| IFIT1, IFI44L, SIGLEC1, HERC6, FAM20A, | 0.885 | 0.876 | 0.894 | 0.919 | 0.858 | 0.980 | 0.945 | 0.877 | 1 |
| RETN, MMP8 | | | | | | | | | |
| RSAD2, IFI27, IFI44L, SIGLEC1, ISG15, | 0.885 | 0.876 | 0.895 | 0.919 | 0.860 | 0.979 | 0.948 | 0.881 | 1 |
| FAM20A, RETN | | | | | | | | | |
| IFI27, IFI44L, SIGLEC1, SLC1A2, IL1R2, OLAH, | 0.885 | 0.877 | 0.894 | 0.927 | 0.877 | 0.977 | 0.951 | 0.901 | 1 |
| MMP8 | | | | | | | | | |
| RSAD2, IFI27, IFIT1, IFI44L, SIGLEC1, ISG15, | 0.885 | 0.876 | 0.894 | 0.915 | 0.850 | 0.980 | 0.945 | 0.874 | 1 |
| FAM20A | | | | | | | | | |
| IFI27, OAS1, IFI44L, HERC6, IL1R2, RETN, | 0.885 | 0.877 | 0.894 | 0.922 | 0.869 | 0.975 | 0.936 | 0.873 | 0.999 |
| MMP8 | | | | | | | | | |
| IFI27, OAS1, IFI44L, ISG15, HERC6, IL1R2, | 0.885 | 0.877 | 0.894 | 0.922 | 0.870 | 0.974 | 0.938 | 0.877 | 0.999 |
| RETN | | | | | | | | | |
| IFI27, OAS1, IFIT1, IFI44L, ISG15, HERC6, | 0.885 | 0.876 | 0.894 | 0.925 | 0.873 | 0.976 | 0.934 | 0.874 | 0.994 |
| OLAH | | | | | | | | | |
| RSAD2, IFI27, OAS1, IFIT1, IFI44L, HERC6, | 0.885 | 0.876 | 0.894 | 0.926 | 0.874 | 0.977 | 0.937 | 0.880 | 0.994 |
| OLAH | | | | | | | | | |
| RSAD2, IFI27, OAS1, IFI44L, SIGLEC1, | 0.885 | 0.876 | 0.895 | 0.918 | 0.858 | 0.979 | 0.947 | 0.879 | 1 |
| FAM20A, RETN | | | | | | | | | |
| RSAD2, IFI27, OAS1, HERC6, SLC1A2, OLAH, | 0.885 | 0.876 | 0.894 | 0.927 | 0.877 | 0.978 | 0.932 | 0.860 | 1 |
| RETN | | | | | | | | | |
| IFI27, OAS1, IFIT1, IFI44L, SIGLEC1, OLAH, | 0.885 | 0.877 | 0.894 | 0.916 | 0.856 | 0.975 | 0.949 | 0.889 | 1 |
| RETN | | | | | | | | | |
| IFI27, OAS1, SLC1A2, OLAH, FAM20A, RETN, | 0.885 | 0.875 | 0.895 | 0.927 | 0.873 | 0.981 | 0.925 | 0.846 | 1 |
| MMP8 | | | | | | | | | |
| RSAD2, IFI27, OAS1, IFI44L, HERC6, IL1R2, | 0.885 | 0.877 | 0.893 | 0.920 | 0.866 | 0.975 | 0.940 | 0.877 | 1 |
| RETN | | | | | | | | | |
| RSAD2, IFI27, IFIT1, IFI44L, HERC6, OLAH, | 0.885 | 0.877 | 0.894 | 0.925 | 0.873 | 0.977 | 0.933 | 0.873 | 0.992 |
| MMP8 | | | | | | | | | |
| IFI27, IFIT1, IFI44L, SIGLEC1, ISG15, OLAH, | 0.885 | 0.876 | 0.894 | 0.920 | 0.865 | 0.976 | 0.942 | 0.887 | 0.998 |
| MMP8 | | | | | | | | | |
| RSAD2, IFI27, OAS1, IFIT1, ISG15, HERC6, | 0.885 | 0.876 | 0.894 | 0.923 | 0.872 | 0.975 | 0.935 | 0.876 | 0.994 |
| OLAH | | | | | | | | | |
| IFI27, OAS1, IFIT1, IFI44L, HERC6, IL1R2, | 0.885 | 0.877 | 0.893 | 0.923 | 0.871 | 0.975 | 0.938 | 0.878 | 0.998 |
| RETN | | | | | | | | | |
| IFIT1, IFI44L, SIGLEC1, IL1R2, FAM20A, | 0.885 | 0.876 | 0.894 | 0.922 | 0.866 | 0.978 | 0.968 | 0.918 | 1 |
| RETN, MMP8 | | | | | | | | | |
| RSAD2, IFI27, IFIT1, ISG15, HERC6, IL1R2, | 0.885 | 0.877 | 0.894 | 0.924 | 0.874 | 0.974 | 0.938 | 0.882 | 0.994 |
| OLAH | | | | | | | | | |
| RSAD2, IFI27, OAS1, SIGLEC1, IL1R2, OLAH, | 0.885 | 0.876 | 0.894 | 0.922 | 0.871 | 0.973 | 0.947 | 0.896 | 0.998 |
| MMP8 | | | | | | | | | |
| OAS1, IFIT1, SIGLEC1, IL1R2, FAM20A, RETN, | 0.885 | 0.876 | 0.894 | 0.922 | 0.865 | 0.978 | 0.968 | 0.918 | 1 |
| MMP8 | | | | | | | | | |
| IFI27, OAS1, IFIT1, IL1R2, OLAH, RETN, MMP8 | 0.885 | 0.876 | 0.894 | 0.921 | 0.864 | 0.977 | 0.954 | 0.902 | 1 |
| IFI27, IFIT1, SIGLEC1, ISG15, IL1R2, OLAH, | 0.885 | 0.877 | 0.893 | 0.919 | 0.863 | 0.974 | 0.947 | 0.891 | 1 |
| RETN | | | | | | | | | |
| IFI27, IFIT1, ISG15, HERC6, IL1R2, OLAH, | 0.885 | 0.877 | 0.893 | 0.924 | 0.875 | 0.974 | 0.942 | 0.888 | 0.997 |
| MMP8 | | | | | | | | | |
| IFI27, IFIT1, IFI44L, SLC1A2, IL1R2, FAM20A, | 0.885 | 0.876 | 0.894 | 0.928 | 0.880 | 0.976 | 0.930 | 0.846 | 1 |
| MMP8 | | | | | | | | | |
| RSAD2, OAS1, IFIT1, SIGLEC1, HERC6, IL1R2, | 0.885 | 0.876 | 0.894 | 0.918 | 0.860 | 0.976 | 0.952 | 0.895 | 1 |
| FAM20A | | | | | | | | | |
| RSAD2, IFI27, OAS1, SIGLEC1, OLAH, RETN, | 0.885 | 0.876 | 0.894 | 0.920 | 0.861 | 0.978 | 0.961 | 0.908 | 1 |
| MMP8 | | | | | | | | | |
| IFI27, IFIT1, IFI44L, HERC6, IL1R2, OLAH, | 0.885 | 0.877 | 0.893 | 0.927 | 0.877 | 0.977 | 0.936 | 0.879 | 0.993 |
| MMP8 | | | | | | | | | |
| IFI27, IFI44L, SIGLEC1, ISG15, IL1R2, RETN, | 0.885 | 0.877 | 0.893 | 0.923 | 0.871 | 0.976 | 0.962 | 0.922 | 1 |
| MMP8 | | | | | | | | | |
| RSAD2, IFI27, IFIT1, IFI44L, SIGLEC1, HERC6, | 0.885 | 0.877 | 0.893 | 0.915 | 0.856 | 0.974 | 0.937 | 0.880 | 0.994 |
| RETN | | | | | | | | | |
| RSAD2, IFI27, ISG15, IL1R2, FAM20A, RETN, | 0.885 | 0.876 | 0.894 | 0.927 | 0.874 | 0.980 | 0.933 | 0.853 | 1 |
| MMP8 | | | | | | | | | |
| IFIT1, SIGLEC1, HERC6, SLC1A2, IL1R2, | 0.885 | 0.876 | 0.894 | 0.928 | 0.882 | 0.974 | 0.959 | 0.895 | 1 |
| FAM20A, MMP8 | | | | | | | | | |
| RSAD2, IFI27, SIGLEC1, ISG15, HERC6, | 0.885 | 0.876 | 0.894 | 0.929 | 0.879 | 0.979 | 0.938 | 0.879 | 0.997 |
| SLC1A2, OLAH | | | | | | | | | |
| IFI27, OAS1, IFIT1, ISG15, HERC6, IL1R2, | 0.885 | 0.876 | 0.894 | 0.923 | 0.873 | 0.974 | 0.939 | 0.882 | 0.996 |
| OLAH | | | | | | | | | |
| RSAD2, IFI27, SIGLEC1, HERC6, SLC1A2, | 0.885 | 0.875 | 0.894 | 0.928 | 0.877 | 0.978 | 0.938 | 0.880 | 0.996 |
| OLAH, MMP8 | | | | | | | | | |
| RSAD2, IFI27, HERC6, SLC1A2, OLAH, RETN, | 0.885 | 0.876 | 0.894 | 0.928 | 0.877 | 0.979 | 0.941 | 0.881 | 1 |
| MMP8 | | | | | | | | | |
| IFI27, IFIT1, SIGLEC1, ISG15, SLC1A2, | 0.885 | 0.876 | 0.894 | 0.926 | 0.871 | 0.981 | 0.938 | 0.862 | 1 |
| FAM20A, RETN | | | | | | | | | |
| IFI27, OAS1, SIGLEC1, ISG15, IL1R2, OLAH, | 0.885 | 0.876 | 0.893 | 0.923 | 0.872 | 0.974 | 0.946 | 0.895 | 0.996 |
| MMP8 | | | | | | | | | |
| RSAD2, IFI27, SIGLEC1, SLC1A2, IL1R2, | 0.885 | 0.876 | 0.894 | 0.927 | 0.878 | 0.977 | 0.946 | 0.891 | 1.000 |
| OLAH, MMP8 | | | | | | | | | |
| IFI27, IFIT1, IFI44L, ISG15, HERC6, IL1R2, | 0.885 | 0.877 | 0.893 | 0.925 | 0.874 | 0.976 | 0.935 | 0.876 | 0.993 |
| OLAH | | | | | | | | | |
| IFIT1, IFI44L, SIGLEC1, ISG15, HERC6, OLAH, | 0.885 | 0.875 | 0.895 | 0.916 | 0.856 | 0.976 | 0.946 | 0.887 | 1 |
| FAM20A | | | | | | | | | |
| RSAD2, IFI27, IFI44L, ISG15, HERC6, IL1R2, | 0.885 | 0.876 | 0.893 | 0.926 | 0.877 | 0.976 | 0.937 | 0.879 | 0.995 |
| OLAH | | | | | | | | | |
| IFI27, IFIT1, SLC1A2, IL1R2, OLAH, FAM20A, | 0.885 | 0.876 | 0.894 | 0.926 | 0.871 | 0.980 | 0.922 | 0.841 | 1 |
| RETN | | | | | | | | | |
| OAS1, IFIT1, SIGLEC1, ISG15, HERC6, IL1R2, | 0.885 | 0.876 | 0.894 | 0.919 | 0.863 | 0.976 | 0.953 | 0.897 | 1 |
| FAM20A | | | | | | | | | |
| IFI27, OAS1, IFIT1, SIGLEC1, ISG15, SLC1A2, | 0.885 | 0.876 | 0.894 | 0.928 | 0.877 | 0.979 | 0.940 | 0.882 | 0.999 |
| OLAH | | | | | | | | | |
| RSAD2, IFI27, IFI44L, ISG15, HERC6, OLAH, | 0.885 | 0.876 | 0.894 | 0.923 | 0.871 | 0.976 | 0.932 | 0.871 | 0.992 |
| MMP8 | | | | | | | | | |
| RSAD2, IFI27, SIGLEC1, ISG15, SLC1A2, | 0.885 | 0.876 | 0.894 | 0.929 | 0.879 | 0.979 | 0.939 | 0.880 | 0.999 |
| OLAH, MMP8 | | | | | | | | | |
| OAS1, SIGLEC1, ISG15, HERC6, IL1R2, | 0.885 | 0.876 | 0.894 | 0.916 | 0.858 | 0.974 | 0.961 | 0.902 | 1 |
| FAM20A, MMP8 | | | | | | | | | |
| IFI27, SIGLEC1, ISG15, HERC6, IL1R2, OLAH, | 0.885 | 0.876 | 0.894 | 0.920 | 0.863 | 0.977 | 0.946 | 0.891 | 1 |
| RETN | | | | | | | | | |
| IFI27, OAS1, SIGLEC1, HERC6, SLC1A2, IL1R2, | 0.885 | 0.876 | 0.893 | 0.928 | 0.881 | 0.975 | 0.945 | 0.887 | 1 |
| RETN | | | | | | | | | |
| OAS1, IFIT1, SIGLEC1, HERC6, OLAH, | 0.885 | 0.875 | 0.894 | 0.916 | 0.858 | 0.975 | 0.953 | 0.899 | 1 |
| FAM20A, MMP8 | | | | | | | | | |
| IFI27, OAS1, IFIT1, HERC6, SLC1A2, OLAH, | 0.885 | 0.875 | 0.894 | 0.926 | 0.876 | 0.977 | 0.935 | 0.869 | 1 |
| RETN | | | | | | | | | |
| RSAD2, IFIT1, IFI44L, SIGLEC1, IL1R2, | 0.885 | 0.876 | 0.894 | 0.922 | 0.866 | 0.978 | 0.966 | 0.915 | 1 |
| FAM20A, MMP8 | | | | | | | | | |
| IFI27, OAS1, IFIT1, SIGLEC1, SLC1A2, OLAH, | 0.885 | 0.876 | 0.894 | 0.927 | 0.876 | 0.979 | 0.945 | 0.888 | 1 |
| MMP8 | | | | | | | | | |
| IFI27, IFIT1, ISG15, HERC6, FAM20A, RETN, | 0.885 | 0.875 | 0.895 | 0.923 | 0.866 | 0.980 | 0.936 | 0.857 | 1 |
| MMP8 | | | | | | | | | |
| IFI27, OAS1, HERC6, SLC1A2, IL1R2, OLAH, | 0.885 | 0.876 | 0.894 | 0.930 | 0.880 | 0.980 | 0.930 | 0.862 | 0.999 |
| RETN | | | | | | | | | |
| RSAD2, IFI27, IFI44L, SIGLEC1, HERC6, IL1R2, | 0.885 | 0.877 | 0.892 | 0.923 | 0.874 | 0.971 | 0.940 | 0.888 | 0.992 |
| MMP8 | | | | | | | | | |
| IFI27, IFI44L, HERC6, SLC1A2, OLAH, | 0.885 | 0.875 | 0.894 | 0.928 | 0.873 | 0.983 | 0.926 | 0.847 | 1 |
| FAM20A, RETN | | | | | | | | | |
| IFI27, IFIT1, SIGLEC1, ISG15, HERC6, IL1R2, | 0.885 | 0.877 | 0.893 | 0.922 | 0.869 | 0.975 | 0.951 | 0.905 | 0.998 |
| RETN | | | | | | | | | |
| IFI27, OAS1, SIGLEC1, ISG15, OLAH, RETN, | 0.885 | 0.875 | 0.894 | 0.920 | 0.861 | 0.979 | 0.960 | 0.907 | 1 |
| MMP8 | | | | | | | | | |
| IFIT1, SIGLEC1, IL1R2, OLAH, FAM20A, | 0.885 | 0.876 | 0.894 | 0.921 | 0.865 | 0.978 | 0.970 | 0.921 | 1 |
| RETN, MMP8 | | | | | | | | | |
| IFI27, IFIT1, IFI44L, SIGLEC1, IL1R2, RETN, | 0.885 | 0.876 | 0.893 | 0.925 | 0.873 | 0.977 | 0.961 | 0.920 | 1 |
| MMP8 | | | | | | | | | |
| IFI27, IFIT1, SIGLEC1, SLC1A2, IL1R2, OLAH, | 0.885 | 0.876 | 0.894 | 0.927 | 0.877 | 0.977 | 0.948 | 0.895 | 1 |
| MMP8 | | | | | | | | | |
| IFI27, OAS1, ISG15, HERC6, SLC1A2, OLAH, | 0.885 | 0.875 | 0.894 | 0.928 | 0.878 | 0.978 | 0.932 | 0.863 | 1 |
| RETN | | | | | | | | | |
| IFI27, IFIT1, SIGLEC1, ISG15, SLC1A2, OLAH, | 0.885 | 0.876 | 0.893 | 0.929 | 0.878 | 0.980 | 0.940 | 0.879 | 1 |
| RETN | | | | | | | | | |
| RSAD2, IFI27, IFIT1, IFI44L, HERC6, IL1R2, | 0.885 | 0.876 | 0.893 | 0.927 | 0.877 | 0.977 | 0.937 | 0.881 | 0.993 |
| OLAH | | | | | | | | | |
| IFI27, IFI44L, ISG15, IL1R2, OLAH, RETN, | 0.885 | 0.876 | 0.894 | 0.923 | 0.868 | 0.977 | 0.951 | 0.897 | 1 |
| MMP8 | | | | | | | | | |
| IFI27, OAS1, SIGLEC1, SLC1A2, OLAH, RETN, | 0.885 | 0.875 | 0.894 | 0.927 | 0.874 | 0.979 | 0.957 | 0.901 | 1 |
| MMP8 | | | | | | | | | |
| IFI27, OAS1, ISG15, HERC6, SLC1A2, OLAH, | 0.885 | 0.875 | 0.894 | 0.928 | 0.879 | 0.978 | 0.923 | 0.853 | 0.993 |
| MMP8 | | | | | | | | | |
| IFI27, OAS1, IFIT1, HERC6, FAM20A, RETN, | 0.885 | 0.875 | 0.895 | 0.924 | 0.866 | 0.982 | 0.934 | 0.853 | 1 |
| MMP8 | | | | | | | | | |
| IFI27, IFIT1, IFI44L, SIGLEC1, ISG15, SLC1A2, | 0.885 | 0.876 | 0.893 | 0.930 | 0.880 | 0.980 | 0.941 | 0.882 | 1 |
| OLAH | | | | | | | | | |
| RSAD2, IFI27, OAS1, IFI44L, SIGLEC1, IL1R2, | 0.885 | 0.876 | 0.893 | 0.922 | 0.870 | 0.974 | 0.949 | 0.899 | 0.999 |
| OLAH | | | | | | | | | |
| RSAD2, IFIT1, IFI44L, SIGLEC1, HERC6, IL1R2, | 0.885 | 0.876 | 0.893 | 0.919 | 0.861 | 0.976 | 0.952 | 0.895 | 1 |
| FAM20A | | | | | | | | | |
| IFI27, OAS1, HERC6, SLC1A2, IL1R2, FAM20A, | 0.885 | 0.875 | 0.894 | 0.931 | 0.884 | 0.977 | 0.925 | 0.839 | 1 |
| MMP8 | | | | | | | | | |
| IFI27, IFIT1, IFI44L, SIGLEC1, SLC1A2, | 0.885 | 0.875 | 0.894 | 0.923 | 0.868 | 0.979 | 0.940 | 0.865 | 1 |
| FAM20A, RETN | | | | | | | | | |
| RSAD2, IFI27, OAS1, IFIT1, SIGLEC1, HERC6, | 0.885 | 0.876 | 0.893 | 0.922 | 0.876 | 0.969 | 0.952 | 0.907 | 0.997 |
| IL1R2 | | | | | | | | | |
| IFIT1, SIGLEC1, ISG15, HERC6, OLAH, | 0.885 | 0.875 | 0.894 | 0.918 | 0.859 | 0.976 | 0.951 | 0.895 | 1 |
| FAM20A, MMP8 | | | | | | | | | |
| IFI27, OAS1, IFIT1, SIGLEC1, IL1R2, RETN, | 0.885 | 0.876 | 0.893 | 0.923 | 0.870 | 0.976 | 0.963 | 0.918 | 1 |
| MMP8 | | | | | | | | | |
| RSAD2, IFI27, SIGLEC1, HERC6, SLC1A2, | 0.885 | 0.875 | 0.894 | 0.927 | 0.878 | 0.977 | 0.941 | 0.885 | 0.998 |
| IL1R2, OLAH | | | | | | | | | |
| RSAD2, IFI27, HERC6, SLC1A2, IL1R2, | 0.885 | 0.876 | 0.893 | 0.930 | 0.884 | 0.977 | 0.926 | 0.840 | 1 |
| FAM20A, MMP8 | | | | | | | | | |
| RSAD2, IFIT1, SIGLEC1, HERC6, IL1R2, OLAH, | 0.884 | 0.875 | 0.894 | 0.918 | 0.859 | 0.977 | 0.952 | 0.897 | 1 |
| FAM20A | | | | | | | | | |
| IFI27, OAS1, IFI44L, SIGLEC1, IL1R2, OLAH, | 0.884 | 0.876 | 0.893 | 0.920 | 0.865 | 0.975 | 0.951 | 0.899 | 1 |
| RETN | | | | | | | | | |
| IFI27, IFIT1, SIGLEC1, HERC6, SLC1A2, | 0.884 | 0.876 | 0.893 | 0.926 | 0.875 | 0.978 | 0.938 | 0.860 | 1 |
| FAM20A, MMP8 | | | | | | | | | |
| IFI27, IFI44L, SIGLEC1, SLC1A2, OLAH, RETN, | 0.884 | 0.875 | 0.894 | 0.928 | 0.875 | 0.981 | 0.959 | 0.908 | 1 |
| MMP8 | | | | | | | | | |
| RSAD2, IFI27, SIGLEC1, ISG15, HERC6, IL1R2, | 0.884 | 0.876 | 0.893 | 0.921 | 0.868 | 0.974 | 0.950 | 0.901 | 0.999 |
| RETN | | | | | | | | | |
| RSAD2, IFIT1, SIGLEC1, ISG15, HERC6, IL1R2, | 0.884 | 0.876 | 0.893 | 0.918 | 0.860 | 0.976 | 0.954 | 0.898 | 1 |
| FAM20A | | | | | | | | | |
| IFIT1, SIGLEC1, SLC1A2, IL1R2, OLAH, | 0.884 | 0.876 | 0.893 | 0.926 | 0.878 | 0.975 | 0.962 | 0.906 | 1 |
| FAM20A, MMP8 | | | | | | | | | |
| RSAD2, IFI27, OAS1, HERC6, SLC1A2, OLAH, | 0.884 | 0.875 | 0.894 | 0.928 | 0.878 | 0.978 | 0.932 | 0.864 | 0.999 |
| MMP8 | | | | | | | | | |
| IFI27, IFI44L, ISG15, SLC1A2, IL1R2, OLAH, | 0.884 | 0.876 | 0.893 | 0.927 | 0.878 | 0.977 | 0.927 | 0.858 | 0.997 |
| MMP8 | | | | | | | | | |
| IFI27, IFI44L, SIGLEC1, SLC1A2, IL1R2, OLAH, | 0.884 | 0.876 | 0.893 | 0.926 | 0.874 | 0.977 | 0.950 | 0.894 | 1 |
| RETN | | | | | | | | | |
| RSAD2, IFI27, OAS1, IFI44L, ISG15, HERC6, | 0.884 | 0.875 | 0.893 | 0.919 | 0.859 | 0.979 | 0.937 | 0.869 | 1 |
| RETN | | | | | | | | | |
| IFI27, OAS1, IFIT1, SLC1A2, OLAH, FAM20A, | 0.884 | 0.875 | 0.894 | 0.924 | 0.867 | 0.981 | 0.925 | 0.847 | 1 |
| RETN | | | | | | | | | |
| IFI27, OAS1, IFI44L, SIGLEC1, ISG15, OLAH, | 0.884 | 0.876 | 0.893 | 0.919 | 0.863 | 0.976 | 0.939 | 0.884 | 0.995 |
| MMP8 | | | | | | | | | |
| RSAD2, IFI27, OAS1, IFIT1, ISG15, IL1R2, | 0.884 | 0.875 | 0.893 | 0.923 | 0.870 | 0.977 | 0.929 | 0.850 | 1 |
| FAM20A | | | | | | | | | |
| RSAD2, IFI27, SIGLEC1, ISG15, SLC1A2, | 0.884 | 0.876 | 0.893 | 0.929 | 0.880 | 0.979 | 0.939 | 0.880 | 0.998 |
| IL1R2, OLAH | | | | | | | | | |
| IFI27, IFIT1, HERC6, SLC1A2, OLAH, RETN, | 0.884 | 0.875 | 0.894 | 0.927 | 0.874 | 0.980 | 0.942 | 0.883 | 1 |
| MMP8 | | | | | | | | | |
| IFI27, IFIT1, IFI44L, SIGLEC1, ISG15, OLAH, | 0.884 | 0.876 | 0.893 | 0.920 | 0.863 | 0.977 | 0.946 | 0.885 | 1 |
| RETN | | | | | | | | | |
| IFIT1, IFI44L, SIGLEC1, HERC6, IL1R2, | 0.884 | 0.876 | 0.893 | 0.918 | 0.861 | 0.976 | 0.952 | 0.895 | 1 |
| FAM20A, RETN | | | | | | | | | |
| IFI27, OAS1, IFIT1, HERC6, SLC1A2, OLAH, | 0.884 | 0.875 | 0.894 | 0.929 | 0.879 | 0.978 | 0.933 | 0.867 | 0.998 |
| MMP8 | | | | | | | | | |
| IFI27, OAS1, IFIT1, SIGLEC1, ISG15, IL1R2, | 0.884 | 0.876 | 0.892 | 0.921 | 0.869 | 0.973 | 0.943 | 0.889 | 0.998 |
| OLAH | | | | | | | | | |
| RSAD2, IFI27, SIGLEC1, ISG15, HERC6, | 0.884 | 0.875 | 0.894 | 0.918 | 0.857 | 0.978 | 0.940 | 0.868 | 1 |
| FAM20A, MMP8 | | | | | | | | | |
| RSAD2, IFI27, IFIT1, SIGLEC1, OLAH, RETN, | 0.884 | 0.875 | 0.893 | 0.919 | 0.860 | 0.978 | 0.962 | 0.911 | 1 |
| MMP8 | | | | | | | | | |
| RSAD2, IFI27, HERC6, SLC1A2, IL1R2, OLAH, | 0.884 | 0.875 | 0.893 | 0.926 | 0.877 | 0.976 | 0.929 | 0.864 | 0.995 |
| MMP8 | | | | | | | | | |
| IFI27, IFIT1, IFI44L, SIGLEC1, IL1R2, OLAH, | 0.884 | 0.876 | 0.893 | 0.920 | 0.865 | 0.975 | 0.952 | 0.899 | 1 |
| RETN | | | | | | | | | |
| IFI27, IFIT1, IFI44L, SIGLEC1, HERC6, IL1R2, | 0.884 | 0.876 | 0.892 | 0.922 | 0.874 | 0.970 | 0.940 | 0.888 | 0.992 |
| MMP8 | | | | | | | | | |
| RSAD2, IFI27, SIGLEC1, ISG15, IL1R2, OLAH, | 0.884 | 0.876 | 0.893 | 0.923 | 0.872 | 0.974 | 0.945 | 0.894 | 0.995 |
| MMP8 | | | | | | | | | |
| IFI27, OAS1, SIGLEC1, SLC1A2, FAM20A, | 0.884 | 0.874 | 0.894 | 0.923 | 0.865 | 0.980 | 0.942 | 0.868 | 1 |
| RETN, MMP8 | | | | | | | | | |
| IFI27, OAS1, IFIT1, SIGLEC1, ISG15, OLAH, | 0.884 | 0.876 | 0.893 | 0.917 | 0.860 | 0.975 | 0.947 | 0.890 | 1 |
| RETN | | | | | | | | | |
| IFI27, IFIT1, IFI44L, SIGLEC1, ISG15, HERC6, | 0.884 | 0.876 | 0.892 | 0.922 | 0.874 | 0.970 | 0.942 | 0.891 | 0.993 |
| IL1R2 | | | | | | | | | |
| IFI27, SIGLEC1, ISG15, HERC6, SLC1A2, | 0.884 | 0.875 | 0.893 | 0.929 | 0.880 | 0.979 | 0.937 | 0.877 | 0.997 |
| IL1R2, OLAH | | | | | | | | | |
| RSAD2, IFI27, SIGLEC1, HERC6, SLC1A2, | 0.884 | 0.875 | 0.893 | 0.928 | 0.878 | 0.978 | 0.946 | 0.890 | 1 |
| OLAH, RETN | | | | | | | | | |
| IFI27, OAS1, IFIT1, IFI44L, SIGLEC1, FAM20A, | 0.884 | 0.875 | 0.894 | 0.914 | 0.848 | 0.979 | 0.947 | 0.880 | 1 |
| MMP8 | | | | | | | | | |
| IFI27, IFI44L, SIGLEC1, ISG15, HERC6, | 0.884 | 0.875 | 0.893 | 0.930 | 0.880 | 0.980 | 0.940 | 0.883 | 0.997 |
| SLC1A2, OLAH | | | | | | | | | |
| RSAD2, IFI27, IFIT1, IFI44L, IL1R2, FAM20A, | 0.884 | 0.875 | 0.894 | 0.923 | 0.869 | 0.977 | 0.930 | 0.852 | 1 |
| RETN | | | | | | | | | |
| IFI27, OAS1, IFIT1, IFI44L, SIGLEC1, IL1R2, | 0.884 | 0.876 | 0.892 | 0.922 | 0.869 | 0.974 | 0.950 | 0.899 | 1 |
| RETN | | | | | | | | | |
| IFI27, OAS1, SIGLEC1, SLC1A2, IL1R2, OLAH, | 0.884 | 0.875 | 0.893 | 0.927 | 0.877 | 0.978 | 0.950 | 0.890 | 1 |
| RETN | | | | | | | | | |
| IFI27, OAS1, SLC1A2, IL1R2, OLAH, FAM20A, | 0.884 | 0.875 | 0.893 | 0.927 | 0.873 | 0.98 | 0.920 | 0.836 | 1 |
| RETN | | | | | | | | | |
| RSAD2, SIGLEC1, HERC6, SLC1A2, IL1R2, | 0.884 | 0.874 | 0.894 | 0.925 | 0.877 | 0.972 | 0.959 | 0.895 | 1 |
| FAM20A, MMP8 | | | | | | | | | |
| IFI27, SIGLEC1, ISG15, SLC1A2, IL1R2, OLAH, | 0.884 | 0.875 | 0.893 | 0.928 | 0.877 | 0.979 | 0.941 | 0.882 | 1 |
| RETN | | | | | | | | | |
| IFI27, IFIT1, ISG15, IL1R2, OLAH, RETN, | 0.884 | 0.875 | 0.893 | 0.920 | 0.864 | 0.976 | 0.951 | 0.898 | 1 |
| MMP8 | | | | | | | | | |
| RSAD2, IFI27, OAS1, IFIT1, OLAH, RETN, | 0.884 | 0.875 | 0.893 | 0.919 | 0.859 | 0.978 | 0.952 | 0.896 | 1 |
| MMP8 | | | | | | | | | |
| RSAD2, IFI27, IFIT1, SIGLEC1, IL1R2, OLAH, | 0.884 | 0.876 | 0.893 | 0.922 | 0.872 | 0.973 | 0.949 | 0.900 | 0.998 |
| MMP8 | | | | | | | | | |
| RSAD2, IFI27, OAS1, IFIT1, SIGLEC1, ISG15, | 0.884 | 0.875 | 0.893 | 0.916 | 0.852 | 0.980 | 0.941 | 0.869 | 1 |
| FAM20A | | | | | | | | | |
| IFI27, OAS1, IFIT1, SIGLEC1, HERC6, SLC1A2, | 0.884 | 0.875 | 0.893 | 0.928 | 0.879 | 0.977 | 0.936 | 0.859 | 1 |
| FAM20A | | | | | | | | | |
| IFI27, OAS1, IFI44L, SLC1A2, OLAH, FAM20A, | 0.884 | 0.875 | 0.894 | 0.927 | 0.870 | 0.984 | 0.923 | 0.843 | 1 |
| RETN | | | | | | | | | |
| OAS1, IFIT1, IFI44L, SIGLEC1, HERC6, OLAH, | 0.884 | 0.875 | 0.893 | 0.916 | 0.856 | 0.975 | 0.942 | 0.882 | 1 |
| FAM20A | | | | | | | | | |
| IFI44L, SIGLEC1, SLC1A2, IL1R2, OLAH, | 0.884 | 0.875 | 0.893 | 0.923 | 0.874 | 0.972 | 0.959 | 0.897 | 1 |
| FAM20A, MMP8 | | | | | | | | | |
| IFIT1, IFI44L, SIGLEC1, SLC1A2, IL1R2, | 0.884 | 0.875 | 0.893 | 0.927 | 0.880 | 0.974 | 0.961 | 0.899 | 1 |
| FAM20A, MMP8 | | | | | | | | | |
| IFI27, HERC6, SLC1A2, IL1R2, FAM20A, RETN, | 0.884 | 0.875 | 0.893 | 0.934 | 0.886 | 0.982 | 0.928 | 0.843 | 1 |
| MMP8 | | | | | | | | | |
| IFI27, IFIT1, IFI44L, SIGLEC1, ISG15, IL1R2, | 0.884 | 0.876 | 0.892 | 0.922 | 0.869 | 0.974 | 0.947 | 0.894 | 0.999 |
| RETN | | | | | | | | | |
| IFI27, OAS1, IFIT1, SIGLEC1, SLC1A2, IL1R2, | 0.884 | 0.875 | 0.893 | 0.927 | 0.877 | 0.977 | 0.946 | 0.889 | 1 |
| OLAH | | | | | | | | | |
| RSAD2, IFI27, IFIT1, SIGLEC1, ISG15, HERC6, | 0.884 | 0.876 | 0.892 | 0.916 | 0.858 | 0.974 | 0.946 | 0.893 | 0.998 |
| RETN | | | | | | | | | |
| IFI27, OAS1, IFIT1, IFI44L, SIGLEC1, ISG15, | 0.884 | 0.876 | 0.893 | 0.918 | 0.861 | 0.975 | 0.938 | 0.880 | 0.996 |
| OLAH | | | | | | | | | |
| IFIT1, SIGLEC1, ISG15, HERC6, FAM20A, | 0.884 | 0.875 | 0.893 | 0.918 | 0.857 | 0.978 | 0.950 | 0.885 | 1 |
| RETN, MMP8 | | | | | | | | | |
| IFI27, OAS1, IFIT1, ISG15, HERC6, SLC1A2, | 0.884 | 0.874 | 0.894 | 0.929 | 0.879 | 0.978 | 0.930 | 0.864 | 0.997 |
| OLAH | | | | | | | | | |
| IFIT1, IFI44L, SIGLEC1, ISG15, HERC6, | 0.884 | 0.875 | 0.893 | 0.917 | 0.856 | 0.978 | 0.943 | 0.881 | 1 |
| FAM20A, RETN | | | | | | | | | |
| RSAD2, IFI27, IFIT1, ISG15, IL1R2, FAM20A, | 0.884 | 0.875 | 0.893 | 0.924 | 0.871 | 0.978 | 0.929 | 0.850 | 1 |
| RETN | | | | | | | | | |
| IFI27, OAS1, IFI44L, HERC6, SLC1A2, IL1R2, | 0.884 | 0.874 | 0.894 | 0.931 | 0.885 | 0.976 | 0.925 | 0.837 | 1 |
| FAM20A | | | | | | | | | |
| IFI27, OAS1, IFIT1, HERC6, SLC1A2, IL1R2, | 0.884 | 0.874 | 0.893 | 0.930 | 0.883 | 0.977 | 0.924 | 0.838 | 1 |
| FAM20A | | | | | | | | | |
| IFI27, IFI44L, SIGLEC1, ISG15, HERC6, IL1R2, | 0.884 | 0.877 | 0.891 | 0.923 | 0.876 | 0.970 | 0.938 | 0.885 | 0.991 |
| MMP8 | | | | | | | | | |
| IFI27, OAS1, HERC6, SLC1A2, IL1R2, FAM20A, | 0.884 | 0.875 | 0.893 | 0.934 | 0.886 | 0.981 | 0.924 | 0.838 | 1 |
| RETN | | | | | | | | | |
| RSAD2, IFI27, SIGLEC1, HERC6, SLC1A2, | 0.884 | 0.874 | 0.894 | 0.926 | 0.873 | 0.979 | 0.945 | 0.871 | 1 |
| FAM20A, RETN | | | | | | | | | |
| RSAD2, IFI27, OAS1, IL1R2, OLAH, RETN, | 0.884 | 0.875 | 0.893 | 0.921 | 0.865 | 0.977 | 0.953 | 0.902 | 1 |
| MMP8 | | | | | | | | | |
| RSAD2, IFI27, IFI44L, SIGLEC1, ISG15, HERC6, | 0.884 | 0.875 | 0.893 | 0.918 | 0.858 | 0.977 | 0.943 | 0.871 | 1 |
| FAM20A | | | | | | | | | |
| RSAD2, IFI27, OAS1, IFI44L, IL1R2, FAM20A, | 0.884 | 0.875 | 0.893 | 0.924 | 0.871 | 0.978 | 0.930 | 0.851 | 1 |
| RETN | | | | | | | | | |
| IFI27, OAS1, SIGLEC1, ISG15, SLC1A2, IL1R2, | 0.884 | 0.875 | 0.893 | 0.930 | 0.881 | 0.978 | 0.941 | 0.883 | 1.000 |
| OLAH | | | | | | | | | |
| RSAD2, IFI27, IFI44L, HERC6, SLC1A2, IL1R2, | 0.884 | 0.875 | 0.893 | 0.928 | 0.880 | 0.976 | 0.925 | 0.838 | 1 |
| FAM20A | | | | | | | | | |
| IFI27, SIGLEC1, ISG15, SLC1A2, OLAH, RETN, | 0.884 | 0.875 | 0.893 | 0.928 | 0.875 | 0.980 | 0.953 | 0.900 | 1 |
| MMP8 | | | | | | | | | |
| RSAD2, IFI27, OAS1, SIGLEC1, ISG15, IL1R2, | 0.884 | 0.875 | 0.892 | 0.921 | 0.869 | 0.973 | 0.945 | 0.891 | 0.998 |
| OLAH | | | | | | | | | |
| IFI27, OAS1, ISG15, HERC6, SLC1A2, IL1R2, | 0.884 | 0.874 | 0.893 | 0.931 | 0.885 | 0.977 | 0.921 | 0.833 | 1 |
| FAM20A | | | | | | | | | |
| IFI27, OAS1, IFIT1, IFI44L, SIGLEC1, OLAH, | 0.884 | 0.875 | 0.892 | 0.920 | 0.864 | 0.976 | 0.950 | 0.900 | 1.000 |
| MMP8 | | | | | | | | | |
| IFI27, OAS1, IFI44L, ISG15, HERC6, RETN, | 0.884 | 0.875 | 0.893 | 0.918 | 0.858 | 0.977 | 0.935 | 0.868 | 1 |
| MMP8 | | | | | | | | | |
| IFI27, IFIT1, IFI44L, IL1R2, OLAH, RETN, | 0.884 | 0.875 | 0.893 | 0.922 | 0.866 | 0.977 | 0.950 | 0.895 | 1 |
| MMP8 | | | | | | | | | |
| RSAD2, IFI27, IFI44L, ISG15, IL1R2, FAM20A, | 0.884 | 0.874 | 0.893 | 0.924 | 0.871 | 0.978 | 0.926 | 0.845 | 1 |
| RETN | | | | | | | | | |
| RSAD2, IFI27, IFIT1, IFI44L, SIGLEC1, IL1R2, | 0.884 | 0.876 | 0.892 | 0.922 | 0.870 | 0.974 | 0.948 | 0.898 | 0.998 |
| OLAH | | | | | | | | | |
| OAS1, SIGLEC1, HERC6, SLC1A2, IL1R2, | 0.884 | 0.874 | 0.893 | 0.923 | 0.874 | 0.971 | 0.960 | 0.896 | 1 |
| FAM20A, MMP8 | | | | | | | | | |
| IFI27, OAS1, IFIT1, SIGLEC1, ISG15, OLAH, | 0.884 | 0.875 | 0.892 | 0.919 | 0.863 | 0.975 | 0.941 | 0.885 | 0.998 |
| MMP8 | | | | | | | | | |
| IFI27, OAS1, IFIT1, ISG15, HERC6, RETN, | 0.884 | 0.874 | 0.893 | 0.917 | 0.858 | 0.976 | 0.935 | 0.868 | 1 |
| MMP8 | | | | | | | | | |
| RSAD2, IFI27, OAS1, SIGLEC1, SLC1A2, | 0.884 | 0.875 | 0.893 | 0.928 | 0.876 | 0.980 | 0.948 | 0.892 | 1 |
| OLAH, MMP8 | | | | | | | | | |
| IFI27, IFI44L, SIGLEC1, HERC6, SLC1A2, | 0.884 | 0.874 | 0.893 | 0.924 | 0.871 | 0.977 | 0.947 | 0.892 | 1 |
| OLAH, RETN | | | | | | | | | |
| IFI27, IFIT1, SLC1A2, IL1R2, FAM20A, RETN, | 0.884 | 0.875 | 0.893 | 0.932 | 0.883 | 0.981 | 0.934 | 0.853 | 1 |
| MMP8 | | | | | | | | | |
| RSAD2, IFI27, IFIT1, SLC1A2, OLAH, FAM20A, | 0.884 | 0.875 | 0.893 | 0.923 | 0.866 | 0.981 | 0.927 | 0.851 | 1 |
| RETN | | | | | | | | | |
| IFI27, IFIT1, SIGLEC1, HERC6, SLC1A2, OLAH, | 0.884 | 0.874 | 0.893 | 0.927 | 0.876 | 0.978 | 0.948 | 0.894 | 1 |
| RETN | | | | | | | | | |
| IFI27, OAS1, IFIT1, ISG15, OLAH, RETN, MMP8 | 0.884 | 0.874 | 0.893 | 0.918 | 0.858 | 0.977 | 0.950 | 0.893 | 1 |
| IFI27, IFIT1, HERC6, SLC1A2, IL1R2, FAM20A, | 0.884 | 0.874 | 0.893 | 0.929 | 0.881 | 0.976 | 0.929 | 0.845 | 1 |
| MMP8 | | | | | | | | | |
| RSAD2, IFIT1, SIGLEC1, HERC6, OLAH, | 0.884 | 0.874 | 0.893 | 0.916 | 0.858 | 0.975 | 0.954 | 0.900 | 1 |
| FAM20A, MMP8 | | | | | | | | | |
| IFI27, IFIT1, SIGLEC1, HERC6, SLC1A2, OLAH, | 0.884 | 0.874 | 0.893 | 0.929 | 0.879 | 0.980 | 0.940 | 0.884 | 0.996 |
| MMP8 | | | | | | | | | |
| IFI27, IFIT1, SIGLEC1, SLC1A2, OLAH, RETN, | 0.884 | 0.874 | 0.893 | 0.928 | 0.877 | 0.980 | 0.955 | 0.901 | 1 |
| MMP8 | | | | | | | | | |
| IFI27, IFIT1, IFI44L, SIGLEC1, HERC6, SLC1A2, | 0.884 | 0.875 | 0.892 | 0.927 | 0.875 | 0.979 | 0.941 | 0.864 | 1 |
| FAM20A | | | | | | | | | |
| RSAD2, IFI27, IFI44L, SLC1A2, IL1R2, | 0.884 | 0.875 | 0.893 | 0.929 | 0.882 | 0.976 | 0.926 | 0.839 | 1 |
| FAM20A, MMP8 | | | | | | | | | |
| IFI27, SIGLEC1, HERC6, SLC1A2, OLAH, | 0.884 | 0.874 | 0.893 | 0.924 | 0.868 | 0.979 | 0.953 | 0.900 | 1 |
| RETN, MMP8 | | | | | | | | | |
| RSAD2, IFI27, IFIT1, ISG15, HERC6, OLAH, | 0.884 | 0.875 | 0.893 | 0.923 | 0.871 | 0.976 | 0.935 | 0.876 | 0.994 |
| MMP8 | | | | | | | | | |
| IFI27, SIGLEC1, HERC6, SLC1A2, IL1R2, | 0.884 | 0.874 | 0.893 | 0.930 | 0.879 | 0.980 | 0.947 | 0.875 | 1 |
| FAM20A, RETN | | | | | | | | | |
| RSAD2, IFI27, OAS1, SLC1A2, OLAH, FAM20A, | 0.884 | 0.874 | 0.893 | 0.925 | 0.869 | 0.982 | 0.920 | 0.836 | 1 |
| RETN | | | | | | | | | |
| RSAD2, IFI27, HERC6, SLC1A2, IL1R2, | 0.884 | 0.875 | 0.893 | 0.933 | 0.885 | 0.981 | 0.925 | 0.840 | 1 |
| FAM20A, RETN | | | | | | | | | |
| RSAD2, IFI27, OAS1, IFIT1, SIGLEC1, IL1R2, | 0.884 | 0.875 | 0.892 | 0.920 | 0.868 | 0.973 | 0.942 | 0.888 | 0.996 |
| OLAH | | | | | | | | | |
| RSAD2, IFI27, IFIT1, SLC1A2, IL1R2, FAM20A, | 0.884 | 0.875 | 0.892 | 0.927 | 0.878 | 0.976 | 0.926 | 0.841 | 1 |
| MMP8 | | | | | | | | | |
| IFIT1, SIGLEC1, HERC6, SLC1A2, OLAH, | 0.884 | 0.873 | 0.894 | 0.926 | 0.877 | 0.976 | 0.948 | 0.879 | 1 |
| FAM20A, MMP8 | | | | | | | | | |
| IFI27, OAS1, IFIT1, SIGLEC1, SLC1A2, | 0.884 | 0.874 | 0.893 | 0.924 | 0.868 | 0.980 | 0.941 | 0.868 | 1 |
| FAM20A, RETN | | | | | | | | | |
| IFI27, OAS1, SIGLEC1, ISG15, HERC6, | 0.884 | 0.874 | 0.894 | 0.915 | 0.855 | 0.976 | 0.940 | 0.868 | 1 |
| FAM20A, MMP8 | | | | | | | | | |
| RSAD2, IFI27, OAS1, IFIT1, IL1R2, OLAH, | 0.884 | 0.875 | 0.892 | 0.922 | 0.870 | 0.973 | 0.946 | 0.894 | 0.997 |
| MMP8 | | | | | | | | | |
| RSAD2, IFI27, OAS1, ISG15, IL1R2, FAM20A, | 0.884 | 0.875 | 0.893 | 0.926 | 0.874 | 0.978 | 0.929 | 0.850 | 1 |
| RETN | | | | | | | | | |
| RSAD2, IFI27, OAS1, SIGLEC1, IL1R2, OLAH, | 0.884 | 0.875 | 0.892 | 0.918 | 0.862 | 0.974 | 0.951 | 0.895 | 1 |
| RETN | | | | | | | | | |
| OAS1, SIGLEC1, HERC6, IL1R2, FAM20A, | 0.884 | 0.874 | 0.893 | 0.919 | 0.861 | 0.976 | 0.963 | 0.907 | 1 |
| RETN, MMP8 | | | | | | | | | |
| RSAD2, IFI27, IFI44L, SIGLEC1, OLAH, RETN, | 0.884 | 0.875 | 0.892 | 0.920 | 0.862 | 0.978 | 0.959 | 0.908 | 1 |
| MMP8 | | | | | | | | | |
| IFI27, OAS1, SIGLEC1, ISG15, HERC6, IL1R2, | 0.884 | 0.875 | 0.892 | 0.921 | 0.874 | 0.968 | 0.949 | 0.902 | 0.996 |
| MMP8 | | | | | | | | | |
| OAS1, IFIT1, SIGLEC1, HERC6, FAM20A, | 0.884 | 0.875 | 0.892 | 0.918 | 0.858 | 0.978 | 0.941 | 0.873 | 1 |
| RETN, MMP8 | | | | | | | | | |
| RSAD2, IFI27, OAS1, IFI44L, SIGLEC1, ISG15, | 0.884 | 0.875 | 0.892 | 0.919 | 0.862 | 0.976 | 0.942 | 0.887 | 0.997 |
| OLAH | | | | | | | | | |
| IFI27, ISG15, SLC1A2, IL1R2, OLAH, RETN, | 0.884 | 0.875 | 0.892 | 0.928 | 0.877 | 0.978 | 0.936 | 0.869 | 1 |
| MMP8 | | | | | | | | | |
| RSAD2, IFI27, OAS1, SIGLEC1, HERC6, | 0.884 | 0.875 | 0.892 | 0.922 | 0.866 | 0.977 | 0.945 | 0.880 | 1 |
| SLC1A2, RETN | | | | | | | | | |
| IFI27, OAS1, IFI44L, SIGLEC1, HERC6, | 0.884 | 0.874 | 0.893 | 0.927 | 0.876 | 0.977 | 0.942 | 0.885 | 0.999 |
| SLC1A2, OLAH | | | | | | | | | |
| IFI44L, SIGLEC1, HERC6, IL1R2, FAM20A, | 0.884 | 0.874 | 0.893 | 0.918 | 0.861 | 0.976 | 0.959 | 0.896 | 1 |
| RETN, MMP8 | | | | | | | | | |
| RSAD2, IFI27, OAS1, SIGLEC1, ISG15, SLC1A2, | 0.884 | 0.875 | 0.892 | 0.929 | 0.878 | 0.980 | 0.943 | 0.886 | 1 |
| OLAH | | | | | | | | | |
| RSAD2, IFI27, OAS1, SIGLEC1, SLC1A2, IL1R2, | 0.884 | 0.875 | 0.892 | 0.925 | 0.874 | 0.976 | 0.950 | 0.894 | 1 |
| OLAH | | | | | | | | | |
| IFIT1, IFI44L, SIGLEC1, HERC6, OLAH, | 0.884 | 0.874 | 0.893 | 0.916 | 0.856 | 0.977 | 0.945 | 0.886 | 1 |
| FAM20A, RETN | | | | | | | | | |
| RSAD2, IFI27, OAS1, IFI44L, SIGLEC1, OLAH, | 0.884 | 0.875 | 0.892 | 0.918 | 0.861 | 0.975 | 0.951 | 0.901 | 1 |
| MMP8 | | | | | | | | | |
| RSAD2, IFI44L, SIGLEC1, IL1R2, OLAH, | 0.884 | 0.874 | 0.893 | 0.918 | 0.861 | 0.975 | 0.963 | 0.908 | 1 |
| FAM20A, MMP8 | | | | | | | | | |
| IFI27, OAS1, IFIT1, SIGLEC1, ISG15, HERC6, | 0.884 | 0.875 | 0.892 | 0.922 | 0.875 | 0.968 | 0.950 | 0.904 | 0.996 |
| IL1R2 | | | | | | | | | |
| IFI27, OAS1, IFI44L, SIGLEC1, ISG15, HERC6, | 0.884 | 0.875 | 0.892 | 0.915 | 0.856 | 0.975 | 0.946 | 0.872 | 1 |
| FAM20A | | | | | | | | | |
| RSAD2, IFI27, IFI44L, HERC6, FAM20A, RETN, | 0.884 | 0.873 | 0.894 | 0.925 | 0.867 | 0.983 | 0.935 | 0.855 | 1 |
| MMP8 | | | | | | | | | |
| IFI27, OAS1, SIGLEC1, ISG15, SLC1A2, OLAH, | 0.884 | 0.875 | 0.892 | 0.929 | 0.879 | 0.980 | 0.943 | 0.883 | 1 |
| RETN | | | | | | | | | |
| RSAD2, IFI27, OAS1, HERC6, SLC1A2, IL1R2, | 0.884 | 0.874 | 0.893 | 0.929 | 0.881 | 0.977 | 0.935 | 0.869 | 1 |
| OLAH | | | | | | | | | |
| IFI27, OAS1, ISG15, IL1R2, OLAH, RETN, | 0.884 | 0.874 | 0.893 | 0.922 | 0.867 | 0.978 | 0.948 | 0.894 | 1 |
| MMP8 | | | | | | | | | |
| IFI27, OAS1, IFI44L, IL1R2, OLAH, RETN, | 0.883 | 0.874 | 0.893 | 0.922 | 0.867 | 0.978 | 0.950 | 0.896 | 1 |
| MMP8 | | | | | | | | | |
| IFI27, OAS1, SIGLEC1, ISG15, HERC6, SLC1A2, | 0.883 | 0.875 | 0.892 | 0.920 | 0.865 | 0.976 | 0.946 | 0.884 | 1 |
| RETN | | | | | | | | | |
| IFI27, OAS1, IFI44L, SIGLEC1, ISG15, HERC6, | 0.883 | 0.876 | 0.891 | 0.922 | 0.876 | 0.968 | 0.939 | 0.886 | 0.992 |
| IL1R2 | | | | | | | | | |
| RSAD2, IFI27, IFIT1, IFI44L, SIGLEC1, HERC6, | 0.883 | 0.876 | 0.891 | 0.921 | 0.872 | 0.970 | 0.941 | 0.890 | 0.993 |
| IL1R2 | | | | | | | | | |
| IFI27, OAS1, IFIT1, IFI44L, OLAH, RETN, | 0.883 | 0.874 | 0.893 | 0.921 | 0.862 | 0.980 | 0.951 | 0.893 | 1 |
| MMP8 | | | | | | | | | |
| IFI27, OAS1, SIGLEC1, HERC6, SLC1A2, RETN, | 0.883 | 0.875 | 0.892 | 0.923 | 0.868 | 0.977 | 0.947 | 0.885 | 1 |
| MMP8 | | | | | | | | | |
| IFI27, OAS1, IFI44L, SIGLEC1, IL1R2, RETN, | 0.883 | 0.875 | 0.892 | 0.923 | 0.871 | 0.975 | 0.961 | 0.919 | 1 |
| MMP8 | | | | | | | | | |
| IFI27, SIGLEC1, HERC6, SLC1A2, IL1R2, | 0.883 | 0.874 | 0.893 | 0.927 | 0.877 | 0.977 | 0.947 | 0.894 | 0.999 |
| OLAH, MMP8 | | | | | | | | | |
| IFI27, IFIT1, IFI44L, HERC6, IL1R2, RETN, | 0.883 | 0.875 | 0.892 | 0.921 | 0.867 | 0.975 | 0.942 | 0.885 | 0.999 |
| MMP8 | | | | | | | | | |
| IFI27, IFIT1, SIGLEC1, SLC1A2, IL1R2, OLAH, | 0.883 | 0.874 | 0.892 | 0.926 | 0.874 | 0.977 | 0.950 | 0.894 | 1 |
| RETN | | | | | | | | | |
| IFI27, OAS1, IFIT1, HERC6, SLC1A2, IL1R2, | 0.883 | 0.874 | 0.893 | 0.927 | 0.879 | 0.976 | 0.934 | 0.868 | 0.999 |
| OLAH | | | | | | | | | |
| RSAD2, IFI27, OAS1, HERC6, SLC1A2, IL1R2, | 0.883 | 0.874 | 0.893 | 0.931 | 0.886 | 0.976 | 0.921 | 0.832 | 1 |
| FAM20A | | | | | | | | | |
| RSAD2, IFI27, IFI44L, SIGLEC1, ISG15, OLAH, | 0.883 | 0.875 | 0.892 | 0.919 | 0.862 | 0.975 | 0.942 | 0.887 | 0.997 |
| MMP8 | | | | | | | | | |
| RSAD2, IFI27, IFI44L, SIGLEC1, IL1R2, OLAH, | 0.883 | 0.875 | 0.892 | 0.920 | 0.865 | 0.975 | 0.953 | 0.901 | 1 |
| RETN | | | | | | | | | |
| IFI27, IFIT1, ISG15, SLC1A2, IL1R2, OLAH, | 0.883 | 0.875 | 0.892 | 0.927 | 0.878 | 0.977 | 0.932 | 0.868 | 0.995 |
| MMP8 | | | | | | | | | |
| IFI27, OAS1, IFI44L, SIGLEC1, ISG15, SLC1A2, | 0.883 | 0.875 | 0.892 | 0.929 | 0.879 | 0.979 | 0.945 | 0.887 | 1 |
| OLAH | | | | | | | | | |
| IFI27, OAS1, IFIT1, SIGLEC1, SLC1A2, OLAH, | 0.883 | 0.874 | 0.892 | 0.926 | 0.873 | 0.978 | 0.949 | 0.889 | 1 |
| RETN | | | | | | | | | |
| IFI27, IFIT1, IFI44L, SIGLEC1, SLC1A2, OLAH, | 0.883 | 0.875 | 0.892 | 0.926 | 0.874 | 0.978 | 0.949 | 0.892 | 1 |
| RETN | | | | | | | | | |
| RSAD2, IFI27, OAS1, ISG15, HERC6, RETN, | 0.883 | 0.874 | 0.893 | 0.919 | 0.860 | 0.979 | 0.936 | 0.868 | 1 |
| MMP8 | | | | | | | | | |
| IFI27, IFI44L, HERC6, SLC1A2, IL1R2, | 0.883 | 0.874 | 0.893 | 0.930 | 0.884 | 0.976 | 0.927 | 0.840 | 1 |
| FAM20A, MMP8 | | | | | | | | | |
| IFI27, IFIT1, IFI44L, SIGLEC1, HERC6, RETN, | 0.883 | 0.874 | 0.892 | 0.912 | 0.850 | 0.973 | 0.940 | 0.885 | 0.995 |
| MMP8 | | | | | | | | | |
| RSAD2, IFI27, IFIT1, HERC6, FAM20A, RETN, | 0.883 | 0.874 | 0.893 | 0.923 | 0.866 | 0.981 | 0.934 | 0.854 | 1 |
| MMP8 | | | | | | | | | |
| IFI27, OAS1, IFIT1, SLC1A2, IL1R2, FAM20A, | 0.883 | 0.874 | 0.892 | 0.926 | 0.878 | 0.975 | 0.927 | 0.843 | 1 |
| MMP8 | | | | | | | | | |
| RSAD2, IFI27, IFIT1, SIGLEC1, HERC6, | 0.883 | 0.874 | 0.893 | 0.927 | 0.877 | 0.977 | 0.939 | 0.882 | 0.996 |
| SLC1A2, OLAH | | | | | | | | | |
| RSAD2, IFI27, SIGLEC1, SLC1A2, FAM20A, | 0.883 | 0.873 | 0.893 | 0.926 | 0.870 | 0.982 | 0.949 | 0.877 | 1 |
| RETN, MMP8 | | | | | | | | | |
| RSAD2, IFI27, IFIT1, SIGLEC1, ISG15, SLC1A2, | 0.883 | 0.875 | 0.892 | 0.929 | 0.879 | 0.979 | 0.938 | 0.878 | 0.998 |
| OLAH | | | | | | | | | |
| IFI27, OAS1, IFI44L, SIGLEC1, ISG15, OLAH, | 0.883 | 0.875 | 0.892 | 0.919 | 0.862 | 0.976 | 0.948 | 0.892 | 1 |
| RETN | | | | | | | | | |
| IFI27, IFI44L, ISG15, HERC6, FAM20A, RETN, | 0.883 | 0.873 | 0.894 | 0.921 | 0.861 | 0.980 | 0.941 | 0.864 | 1 |
| MMP8 | | | | | | | | | |
| OAS1, IFIT1, SIGLEC1, SLC1A2, IL1R2, | 0.883 | 0.874 | 0.892 | 0.927 | 0.880 | 0.974 | 0.960 | 0.898 | 1 |
| FAM20A, MMP8 | | | | | | | | | |
| RSAD2, IFI27, SIGLEC1, SLC1A2, OLAH, | 0.883 | 0.874 | 0.893 | 0.927 | 0.874 | 0.980 | 0.957 | 0.903 | 1 |
| RETN, MMP8 | | | | | | | | | |
| RSAD2, IFI27, SIGLEC1, ISG15, OLAH, RETN, | 0.883 | 0.874 | 0.892 | 0.920 | 0.861 | 0.979 | 0.957 | 0.903 | 1 |
| MMP8 | | | | | | | | | |
| RSAD2, IFI27, IFIT1, HERC6, IL1R2, RETN, | 0.883 | 0.874 | 0.892 | 0.920 | 0.866 | 0.974 | 0.943 | 0.889 | 0.998 |
| MMP8 | | | | | | | | | |
| RSAD2, IFI27, HERC6, SLC1A2, IL1R2, OLAH, | 0.883 | 0.874 | 0.893 | 0.927 | 0.876 | 0.977 | 0.935 | 0.870 | 1 |
| RETN | | | | | | | | | |
| RSAD2, IFI27, OAS1, IFIT1, IFI44L, HERC6, | 0.883 | 0.874 | 0.892 | 0.916 | 0.854 | 0.977 | 0.934 | 0.861 | 1 |
| RETN | | | | | | | | | |
| RSAD2, IFI27, OAS1, IFIT1, SIGLEC1, SLC1A2, | 0.883 | 0.874 | 0.892 | 0.928 | 0.876 | 0.979 | 0.947 | 0.891 | 1 |
| OLAH | | | | | | | | | |
| RSAD2, IFI27, OAS1, IFIT1, SIGLEC1, OLAH, | 0.883 | 0.874 | 0.892 | 0.916 | 0.859 | 0.974 | 0.950 | 0.892 | 1 |
| RETN | | | | | | | | | |
| RSAD2, SIGLEC1, SLC1A2, IL1R2, OLAH, | 0.883 | 0.874 | 0.892 | 0.924 | 0.875 | 0.973 | 0.960 | 0.900 | 1 |
| FAM20A, MMP8 | | | | | | | | | |
| IFI27, IFIT1, IFI44L, SIGLEC1, SLC1A2, OLAH, | 0.883 | 0.874 | 0.892 | 0.927 | 0.875 | 0.978 | 0.948 | 0.894 | 1 |
| MMP8 | | | | | | | | | |
| RSAD2, SIGLEC1, ISG15, IL1R2, OLAH, | 0.883 | 0.874 | 0.892 | 0.918 | 0.861 | 0.975 | 0.964 | 0.909 | 1 |
| FAM20A, MMP8 | | | | | | | | | |
| RSAD2, IFI27, IFIT1, IFI44L, ISG15, HERC6, | 0.883 | 0.874 | 0.892 | 0.924 | 0.871 | 0.976 | 0.934 | 0.875 | 0.993 |
| OLAH | | | | | | | | | |
| IFI27, OAS1, IFIT1, SIGLEC1, ISG15, FAM20A, | 0.883 | 0.873 | 0.893 | 0.916 | 0.853 | 0.980 | 0.942 | 0.871 | 1 |
| MMP8 | | | | | | | | | |
| RSAD2, OAS1, IFI44L, SIGLEC1, IL1R2, | 0.883 | 0.874 | 0.892 | 0.918 | 0.861 | 0.975 | 0.961 | 0.903 | 1 |
| FAM20A, MMP8 | | | | | | | | | |
| RSAD2, SIGLEC1, HERC6, IL1R2, FAM20A, | 0.883 | 0.874 | 0.892 | 0.921 | 0.865 | 0.977 | 0.959 | 0.898 | 1 |
| RETN, MMP8 | | | | | | | | | |
| IFI27, IFI44L, ISG15, HERC6, IL1R2, RETN, | 0.883 | 0.875 | 0.892 | 0.923 | 0.870 | 0.975 | 0.938 | 0.878 | 0.998 |
| MMP8 | | | | | | | | | |
| RSAD2, IFI27, IFIT1, SIGLEC1, SLC1A2, OLAH, | 0.883 | 0.874 | 0.892 | 0.927 | 0.876 | 0.978 | 0.941 | 0.883 | 0.999 |
| MMP8 | | | | | | | | | |
| RSAD2, IFI27, OAS1, IFIT1, HERC6, SLC1A2, | 0.883 | 0.874 | 0.892 | 0.928 | 0.878 | 0.978 | 0.937 | 0.873 | 1 |
| OLAH | | | | | | | | | |
| IFI27, ISG15, HERC6, SLC1A2, OLAH, RETN, | 0.883 | 0.874 | 0.892 | 0.927 | 0.876 | 0.979 | 0.941 | 0.881 | 1 |
| MMP8 | | | | | | | | | |
| IFI27, IFIT1, SIGLEC1, ISG15, HERC6, SLC1A2, | 0.883 | 0.874 | 0.892 | 0.927 | 0.876 | 0.977 | 0.943 | 0.867 | 1 |
| FAM20A | | | | | | | | | |
| IFI27, OAS1, IFI44L, SIGLEC1, HERC6, | 0.883 | 0.875 | 0.892 | 0.924 | 0.870 | 0.977 | 0.941 | 0.878 | 1 |
| SLC1A2, RETN | | | | | | | | | |
| IFIT1, SIGLEC1, ISG15, HERC6, IL1R2, | 0.883 | 0.874 | 0.892 | 0.918 | 0.860 | 0.975 | 0.953 | 0.897 | 1 |
| FAM20A, RETN | | | | | | | | | |
| IFI27, IFIT1, SIGLEC1, HERC6, SLC1A2, IL1R2, | 0.883 | 0.874 | 0.892 | 0.928 | 0.878 | 0.977 | 0.943 | 0.889 | 0.998 |
| OLAH | | | | | | | | | |
| OAS1, IFIT1, SIGLEC1, HERC6, IL1R2, | 0.883 | 0.874 | 0.892 | 0.919 | 0.861 | 0.976 | 0.953 | 0.897 | 1 |
| FAM20A, RETN | | | | | | | | | |
| IFI27, OAS1, SIGLEC1, ISG15, IL1R2, OLAH, | 0.883 | 0.874 | 0.892 | 0.919 | 0.864 | 0.975 | 0.950 | 0.895 | 1 |
| RETN | | | | | | | | | |
| IFI27, IFIT1, IFI44L, ISG15, IL1R2, OLAH, | 0.883 | 0.874 | 0.892 | 0.920 | 0.867 | 0.972 | 0.939 | 0.883 | 0.995 |
| MMP8 | | | | | | | | | |
| RSAD2, IFI27, IFI44L, SIGLEC1, HERC6, | 0.883 | 0.874 | 0.892 | 0.926 | 0.875 | 0.977 | 0.942 | 0.886 | 0.999 |
| SLC1A2, OLAH | | | | | | | | | |
| RSAD2, IFI27, IFIT1, IL1R2, OLAH, RETN, | 0.883 | 0.874 | 0.892 | 0.921 | 0.866 | 0.977 | 0.952 | 0.900 | 1 |
| MMP8 | | | | | | | | | |
| RSAD2, IFI27, IFI44L, SLC1A2, OLAH, | 0.883 | 0.874 | 0.892 | 0.925 | 0.868 | 0.982 | 0.924 | 0.845 | 1 |
| FAM20A, RETN | | | | | | | | | |
| IFI27, OAS1, IFI44L, SIGLEC1, HERC6, | 0.883 | 0.874 | 0.892 | 0.914 | 0.854 | 0.973 | 0.943 | 0.868 | 1 |
| FAM20A, MMP8 | | | | | | | | | |
| OAS1, SIGLEC1, ISG15, IL1R2, OLAH, | 0.883 | 0.874 | 0.892 | 0.916 | 0.858 | 0.974 | 0.964 | 0.909 | 1 |
| FAM20A, MMP8 | | | | | | | | | |
| RSAD2, IFI27, SLC1A2, IL1R2, FAM20A, RETN, | 0.883 | 0.874 | 0.892 | 0.929 | 0.881 | 0.978 | 0.928 | 0.843 | 1 |
| MMP8 | | | | | | | | | |
| IFI27, OAS1, IFIT1, ISG15, SLC1A2, OLAH, | 0.883 | 0.874 | 0.892 | 0.926 | 0.874 | 0.978 | 0.930 | 0.863 | 0.998 |
| MMP8 | | | | | | | | | |
| IFI27, IFIT1, IFI44L, HERC6, SLC1A2, IL1R2, | 0.883 | 0.874 | 0.892 | 0.929 | 0.881 | 0.977 | 0.927 | 0.842 | 1 |
| FAM20A | | | | | | | | | |
| IFI27, IFI44L, HERC6, SLC1A2, OLAH, RETN, | 0.883 | 0.873 | 0.892 | 0.924 | 0.870 | 0.978 | 0.940 | 0.878 | 1 |
| MMP8 | | | | | | | | | |
| RSAD2, IFI27, IFI44L, SIGLEC1, ISG15, | 0.883 | 0.875 | 0.891 | 0.931 | 0.881 | 0.981 | 0.943 | 0.886 | 1 |
| SLC1A2, OLAH | | | | | | | | | |
| RSAD2, IFI27, IFIT1, SLC1A2, IL1R2, OLAH, | 0.883 | 0.874 | 0.892 | 0.927 | 0.877 | 0.976 | 0.933 | 0.869 | 0.996 |
| MMP8 | | | | | | | | | |
| IFI27, IFI44L, SIGLEC1, HERC6, SLC1A2, | 0.883 | 0.874 | 0.892 | 0.929 | 0.879 | 0.979 | 0.945 | 0.890 | 0.999 |
| OLAH, MMP8 | | | | | | | | | |
| IFI27, OAS1, IFI44L, HERC6, SLC1A2, OLAH, | 0.883 | 0.874 | 0.892 | 0.928 | 0.878 | 0.978 | 0.930 | 0.861 | 1.000 |
| RETN | | | | | | | | | |
| IFIT1, SIGLEC1, HERC6, IL1R2, OLAH, | 0.883 | 0.874 | 0.892 | 0.919 | 0.861 | 0.976 | 0.952 | 0.895 | 1 |
| FAM20A, RETN | | | | | | | | | |
| RSAD2, IFI27, IFI44L, SIGLEC1, HERC6, | 0.883 | 0.874 | 0.892 | 0.918 | 0.859 | 0.977 | 0.941 | 0.869 | 1 |
| FAM20A, MMP8 | | | | | | | | | |
| IFI27, IFI44L, SLC1A2, IL1R2, OLAH, RETN, | 0.883 | 0.874 | 0.892 | 0.926 | 0.874 | 0.978 | 0.941 | 0.878 | 1 |
| MMP8 | | | | | | | | | |
| RSAD2, OAS1, SIGLEC1, ISG15, IL1R2, | 0.883 | 0.874 | 0.892 | 0.918 | 0.860 | 0.975 | 0.962 | 0.903 | 1 |
| FAM20A, MMP8 | | | | | | | | | |
| RSAD2, OAS1, SIGLEC1, IL1R2, FAM20A, | 0.883 | 0.874 | 0.892 | 0.920 | 0.863 | 0.977 | 0.962 | 0.907 | 1 |
| RETN, MMP8 | | | | | | | | | |
| IFI27, OAS1, ISG15, HERC6, SLC1A2, IL1R2, | 0.883 | 0.874 | 0.892 | 0.927 | 0.878 | 0.977 | 0.928 | 0.862 | 0.995 |
| OLAH | | | | | | | | | |
| RSAD2, IFI27, ISG15, HERC6, SLC1A2, IL1R2, | 0.883 | 0.874 | 0.892 | 0.930 | 0.883 | 0.978 | 0.924 | 0.836 | 1 |
| FAM20A | | | | | | | | | |
| IFIT1, IFI44L, SIGLEC1, HERC6, SLC1A2, | 0.883 | 0.873 | 0.893 | 0.924 | 0.873 | 0.976 | 0.930 | 0.858 | 1 |
| OLAH, FAM20A | | | | | | | | | |
| RSAD2, IFI27, OAS1, IFI44L, SIGLEC1, OLAH, | 0.883 | 0.874 | 0.891 | 0.916 | 0.856 | 0.975 | 0.954 | 0.897 | 1 |
| RETN | | | | | | | | | |
| RSAD2, IFI27, OAS1, SIGLEC1, ISG15, OLAH, | 0.883 | 0.874 | 0.892 | 0.919 | 0.863 | 0.974 | 0.941 | 0.884 | 0.998 |
| MMP8 | | | | | | | | | |
| RSAD2, IFI27, OAS1, SIGLEC1, ISG15, HERC6, | 0.883 | 0.874 | 0.891 | 0.922 | 0.875 | 0.969 | 0.953 | 0.909 | 0.998 |
| IL1R2 | | | | | | | | | |
| IFI44L, SIGLEC1, ISG15, IL1R2, FAM20A, | 0.883 | 0.874 | 0.892 | 0.916 | 0.857 | 0.975 | 0.963 | 0.908 | 1 |
| RETN, MMP8 | | | | | | | | | |
| RSAD2, IFI27, SIGLEC1, SLC1A2, IL1R2, | 0.883 | 0.874 | 0.892 | 0.927 | 0.875 | 0.978 | 0.947 | 0.889 | 1 |
| OLAH, RETN | | | | | | | | | |
| IFI44L, SIGLEC1, ISG15, SLC1A2, IL1R2, | 0.883 | 0.874 | 0.891 | 0.925 | 0.877 | 0.973 | 0.959 | 0.893 | 1 |
| FAM20A, MMP8 | | | | | | | | | |
| RSAD2, IFI27, IFIT1, IFI44L, SIGLEC1, OLAH, | 0.883 | 0.874 | 0.891 | 0.916 | 0.857 | 0.976 | 0.951 | 0.896 | 1 |
| RETN | | | | | | | | | |
| IFI27, SIGLEC1, ISG15, HERC6, SLC1A2, | 0.883 | 0.873 | 0.892 | 0.928 | 0.877 | 0.979 | 0.939 | 0.877 | 1 |
| OLAH, RETN | | | | | | | | | |
| IFI27, OAS1, IFI44L, SIGLEC1, SLC1A2, OLAH, | 0.883 | 0.874 | 0.891 | 0.927 | 0.876 | 0.978 | 0.948 | 0.893 | 1 |
| MMP8 | | | | | | | | | |
| IFI27, OAS1, SIGLEC1, ISG15, SLC1A2, | 0.883 | 0.873 | 0.892 | 0.923 | 0.866 | 0.979 | 0.940 | 0.863 | 1 |
| FAM20A, RETN | | | | | | | | | |
| RSAD2, IFI27, OAS1, SIGLEC1, IL1R2, RETN, | 0.883 | 0.873 | 0.892 | 0.922 | 0.868 | 0.976 | 0.965 | 0.920 | 1 |
| MMP8 | | | | | | | | | |
| SIGLEC1, ISG15, SLC1A2, IL1R2, OLAH, | 0.883 | 0.874 | 0.892 | 0.924 | 0.875 | 0.973 | 0.958 | 0.892 | 1 |
| FAM20A, MMP8 | | | | | | | | | |
| IFIT1, SIGLEC1, ISG15, IL1R2, FAM20A, RETN, | 0.883 | 0.874 | 0.892 | 0.922 | 0.866 | 0.979 | 0.967 | 0.916 | 1 |
| MMP8 | | | | | | | | | |
| RSAD2, IFI27, IFIT1, HERC6, SLC1A2, IL1R2, | 0.883 | 0.873 | 0.892 | 0.931 | 0.883 | 0.978 | 0.927 | 0.842 | 1 |
| FAM20A | | | | | | | | | |
| RSAD2, IFI27, OAS1, IFIT1, SIGLEC1, OLAH, | 0.883 | 0.874 | 0.891 | 0.919 | 0.863 | 0.974 | 0.942 | 0.887 | 0.997 |
| MMP8 | | | | | | | | | |
| RSAD2, IFI27, ISG15, HERC6, SLC1A2, OLAH, | 0.883 | 0.874 | 0.892 | 0.927 | 0.876 | 0.978 | 0.933 | 0.866 | 0.999 |
| RETN | | | | | | | | | |
| RSAD2, IFI27, SIGLEC1, ISG15, SLC1A2, | 0.883 | 0.874 | 0.891 | 0.929 | 0.878 | 0.980 | 0.941 | 0.881 | 1 |
| OLAH, RETN | | | | | | | | | |
| RSAD2, IFI27, IFIT1, SIGLEC1, IL1R2, OLAH, | 0.883 | 0.874 | 0.891 | 0.919 | 0.863 | 0.975 | 0.948 | 0.892 | 1 |
| RETN | | | | | | | | | |
| RSAD2, IFI27, ISG15, HERC6, SLC1A2, IL1R2, | 0.883 | 0.874 | 0.891 | 0.929 | 0.879 | 0.978 | 0.927 | 0.860 | 0.995 |
| OLAH | | | | | | | | | |
| IFI27, OAS1, IFIT1, IFI44L, SIGLEC1, SLC1A2, | 0.883 | 0.874 | 0.891 | 0.927 | 0.876 | 0.978 | 0.948 | 0.892 | 1 |
| OLAH | | | | | | | | | |
| RSAD2, IFI27, IFI44L, SIGLEC1, ISG15, OLAH, | 0.883 | 0.874 | 0.891 | 0.920 | 0.863 | 0.978 | 0.948 | 0.891 | 1 |
| RETN | | | | | | | | | |
| IFI27, IFIT1, IFI44L, ISG15, OLAH, RETN, | 0.883 | 0.873 | 0.892 | 0.919 | 0.859 | 0.978 | 0.949 | 0.890 | 1 |
| MMP8 | | | | | | | | | |
| IFI27, OAS1, IFIT1, IFI44L, SIGLEC1, IL1R2, | 0.883 | 0.875 | 0.890 | 0.923 | 0.875 | 0.970 | 0.947 | 0.898 | 0.995 |
| MMP8 | | | | | | | | | |
| RSAD2, IFI27, IFIT1, SIGLEC1, SLC1A2, IL1R2, | 0.883 | 0.874 | 0.891 | 0.927 | 0.877 | 0.978 | 0.945 | 0.889 | 1.000 |
| OLAH | | | | | | | | | |
| IFI27, IFIT1, IFI44L, SIGLEC1, HERC6, SLC1A2, | 0.883 | 0.873 | 0.892 | 0.927 | 0.877 | 0.978 | 0.942 | 0.887 | 0.998 |
| OLAH | | | | | | | | | |
| RSAD2, IFIT1, SIGLEC1, IL1R2, FAM20A, | 0.883 | 0.874 | 0.891 | 0.922 | 0.866 | 0.979 | 0.968 | 0.918 | 1 |
| RETN, MMP8 | | | | | | | | | |
| RSAD2, IFI27, OAS1, ISG15, HERC6, SLC1A2, | 0.883 | 0.873 | 0.892 | 0.929 | 0.879 | 0.979 | 0.932 | 0.864 | 0.999 |
| OLAH | | | | | | | | | |
| IFI27, IFI44L, ISG15, SLC1A2, OLAH, RETN, | 0.883 | 0.873 | 0.892 | 0.926 | 0.873 | 0.979 | 0.939 | 0.875 | 1 |
| MMP8 | | | | | | | | | |
| IFI27, OAS1, IFI44L, SIGLEC1, SLC1A2, OLAH, | 0.883 | 0.874 | 0.891 | 0.926 | 0.874 | 0.978 | 0.947 | 0.884 | 1 |
| RETN | | | | | | | | | |
| OAS1, IFI44L, SIGLEC1, IL1R2, OLAH, | 0.883 | 0.873 | 0.892 | 0.918 | 0.861 | 0.975 | 0.963 | 0.908 | 1 |
| FAM20A, MMP8 | | | | | | | | | |
| IFI27, IFIT1, ISG15, HERC6, SLC1A2, OLAH, | 0.883 | 0.873 | 0.892 | 0.928 | 0.877 | 0.978 | 0.928 | 0.861 | 0.995 |
| MMP8 | | | | | | | | | |
| SIGLEC1, HERC6, SLC1A2, IL1R2, OLAH, | 0.883 | 0.873 | 0.892 | 0.922 | 0.872 | 0.972 | 0.960 | 0.898 | 1 |
| FAM20A, MMP8 | | | | | | | | | |
| RSAD2, IFI27, OAS1, SIGLEC1, SLC1A2, | 0.883 | 0.874 | 0.891 | 0.925 | 0.872 | 0.978 | 0.949 | 0.887 | 1 |
| OLAH, RETN | | | | | | | | | |
| RSAD2, IFI27, IFIT1, IFI44L, HERC6, IL1R2, | 0.883 | 0.874 | 0.891 | 0.920 | 0.867 | 0.973 | 0.936 | 0.876 | 0.995 |
| RETN | | | | | | | | | |
| IFI27, OAS1, IFIT1, IFI44L, IL1R2, OLAH, | 0.883 | 0.874 | 0.891 | 0.921 | 0.869 | 0.973 | 0.942 | 0.889 | 0.995 |
| MMP8 | | | | | | | | | |
| IFI27, ISG15, HERC6, SLC1A2, IL1R2, OLAH, | 0.883 | 0.874 | 0.891 | 0.928 | 0.879 | 0.977 | 0.927 | 0.860 | 0.994 |
| MMP8 | | | | | | | | | |
| IFI27, IFI44L, ISG15, HERC6, SLC1A2, IL1R2, | 0.882 | 0.873 | 0.892 | 0.929 | 0.882 | 0.976 | 0.928 | 0.843 | 1 |
| FAM20A | | | | | | | | | |
| RSAD2, IFI27, IFIT1, IFI44L, SIGLEC1, OLAH, | 0.882 | 0.874 | 0.891 | 0.919 | 0.862 | 0.975 | 0.949 | 0.898 | 1.000 |
| MMP8 | | | | | | | | | |
| RSAD2, IFI27, ISG15, SLC1A2, IL1R2, OLAH, | 0.882 | 0.874 | 0.891 | 0.929 | 0.879 | 0.978 | 0.928 | 0.861 | 0.996 |
| MMP8 | | | | | | | | | |
| IFI27, OAS1, IFI44L, SLC1A2, IL1R2, FAM20A, | 0.882 | 0.873 | 0.892 | 0.929 | 0.882 | 0.976 | 0.926 | 0.839 | 1 |
| MMP8 | | | | | | | | | |
| IFI27, IFIT1, ISG15, SLC1A2, IL1R2, FAM20A, | 0.882 | 0.873 | 0.891 | 0.928 | 0.880 | 0.976 | 0.927 | 0.842 | 1 |
| MMP8 | | | | | | | | | |
| IFI27, OAS1, IFIT1, SLC1A2, IL1R2, OLAH, | 0.882 | 0.874 | 0.891 | 0.927 | 0.878 | 0.976 | 0.936 | 0.872 | 1 |
| MMP8 | | | | | | | | | |
| IFIT1, SIGLEC1, ISG15, HERC6, OLAH, | 0.882 | 0.873 | 0.892 | 0.916 | 0.855 | 0.976 | 0.949 | 0.892 | 1 |
| FAM20A, RETN | | | | | | | | | |
| RSAD2, IFI27, IFI44L, HERC6, IL1R2, RETN, | 0.882 | 0.874 | 0.891 | 0.920 | 0.867 | 0.974 | 0.933 | 0.869 | 0.996 |
| MMP8 | | | | | | | | | |
| IFI27, SIGLEC1, ISG15, HERC6, IL1R2, RETN, | 0.882 | 0.874 | 0.891 | 0.921 | 0.866 | 0.976 | 0.963 | 0.923 | 1 |
| MMP8 | | | | | | | | | |
| RSAD2, IFIT1, SIGLEC1, SLC1A2, IL1R2, | 0.882 | 0.873 | 0.891 | 0.928 | 0.882 | 0.974 | 0.960 | 0.898 | 1 |
| FAM20A, MMP8 | | | | | | | | | |
| RSAD2, IFI27, IFIT1, SIGLEC1, ISG15, IL1R2, | 0.882 | 0.874 | 0.891 | 0.921 | 0.868 | 0.973 | 0.945 | 0.891 | 0.998 |
| OLAH | | | | | | | | | |
| RSAD2, IFI27, IFIT1, IFI44L, IL1R2, OLAH, | 0.882 | 0.874 | 0.891 | 0.922 | 0.870 | 0.973 | 0.942 | 0.889 | 0.995 |
| MMP8 | | | | | | | | | |
| RSAD2, IFI27, OAS1, IFI44L, SIGLEC1, IL1R2, | 0.882 | 0.874 | 0.891 | 0.916 | 0.861 | 0.972 | 0.958 | 0.908 | 1 |
| RETN | | | | | | | | | |
| IFI27, IFIT1, IFI44L, SIGLEC1, ISG15, SLC1A2, | 0.882 | 0.873 | 0.891 | 0.922 | 0.866 | 0.978 | 0.940 | 0.864 | 1 |
| FAM20A | | | | | | | | | |
| RSAD2, IFI27, IFI44L, SIGLEC1, IL1R2, RETN, | 0.882 | 0.874 | 0.891 | 0.922 | 0.869 | 0.976 | 0.960 | 0.918 | 1 |
| MMP8 | | | | | | | | | |
| RSAD2, IFI27, ISG15, HERC6, SLC1A2, OLAH, | 0.882 | 0.873 | 0.891 | 0.928 | 0.878 | 0.978 | 0.925 | 0.857 | 0.993 |
| MMP8 | | | | | | | | | |
| IFI27, SIGLEC1, SLC1A2, IL1R2, OLAH, RETN, | 0.882 | 0.873 | 0.892 | 0.926 | 0.874 | 0.978 | 0.958 | 0.909 | 1 |
| MMP8 | | | | | | | | | |
| RSAD2, IFI27, IFIT1, HERC6, SLC1A2, OLAH, | 0.882 | 0.873 | 0.892 | 0.925 | 0.875 | 0.976 | 0.935 | 0.870 | 1 |
| RETN | | | | | | | | | |
| IFI27, IFI44L, SIGLEC1, HERC6, SLC1A2, | 0.882 | 0.873 | 0.892 | 0.927 | 0.877 | 0.977 | 0.946 | 0.893 | 0.998 |
| IL1R2, OLAH | | | | | | | | | |
| RSAD2, IFI27, OAS1, IFI44L, SIGLEC1, | 0.882 | 0.874 | 0.891 | 0.926 | 0.874 | 0.978 | 0.953 | 0.900 | 1 |
| SLC1A2, OLAH | | | | | | | | | |
| IFI27, IFIT1, ISG15, HERC6, SLC1A2, IL1R2, | 0.882 | 0.873 | 0.891 | 0.927 | 0.877 | 0.976 | 0.927 | 0.860 | 0.994 |
| OLAH | | | | | | | | | |
| SIGLEC1, ISG15, IL1R2, OLAH, FAM20A, | 0.882 | 0.873 | 0.892 | 0.915 | 0.855 | 0.975 | 0.965 | 0.911 | 1 |
| RETN, MMP8 | | | | | | | | | |
| IFI27, IFI44L, ISG15, SLC1A2, IL1R2, FAM20A, | 0.882 | 0.873 | 0.891 | 0.927 | 0.877 | 0.977 | 0.924 | 0.839 | 1 |
| RETN | | | | | | | | | |
| RSAD2, IFI27, IFIT1, IFI44L, SIGLEC1, | 0.882 | 0.873 | 0.892 | 0.915 | 0.849 | 0.980 | 0.946 | 0.877 | 1 |
| FAM20A, MMP8 | | | | | | | | | |
| OAS1, SIGLEC1, SLC1A2, IL1R2, OLAH, | 0.882 | 0.873 | 0.891 | 0.925 | 0.876 | 0.974 | 0.959 | 0.898 | 1 |
| FAM20A, MMP8 | | | | | | | | | |
| IFI27, OAS1, IFI44L, HERC6, SLC1A2, OLAH, | 0.882 | 0.873 | 0.892 | 0.928 | 0.878 | 0.977 | 0.929 | 0.861 | 0.997 |
| MMP8 | | | | | | | | | |
| IFI27, OAS1, IFIT1, ISG15, IL1R2, OLAH, MMP8 | 0.882 | 0.874 | 0.891 | 0.921 | 0.870 | 0.972 | 0.946 | 0.894 | 0.998 |
| RSAD2, IFI27, IFIT1, ISG15, SLC1A2, OLAH, | 0.882 | 0.873 | 0.891 | 0.926 | 0.874 | 0.978 | 0.929 | 0.863 | 0.996 |
| MMP8 | | | | | | | | | |
| IFI27, IFIT1, IFI44L, ISG15, SLC1A2, OLAH, | 0.882 | 0.873 | 0.891 | 0.927 | 0.875 | 0.979 | 0.927 | 0.859 | 0.996 |
| MMP8 | | | | | | | | | |
| IFI27, OAS1, IFI44L, ISG15, OLAH, RETN, | 0.882 | 0.873 | 0.891 | 0.919 | 0.860 | 0.978 | 0.948 | 0.891 | 1 |
| MMP8 | | | | | | | | | |
| RSAD2, IFI27, OAS1, IFIT1, IFI44L, SIGLEC1, | 0.882 | 0.874 | 0.891 | 0.919 | 0.862 | 0.975 | 0.951 | 0.901 | 1 |
| OLAH | | | | | | | | | |
| IFIT1, SIGLEC1, ISG15, SLC1A2, OLAH, | 0.882 | 0.873 | 0.891 | 0.921 | 0.868 | 0.974 | 0.947 | 0.884 | 1 |
| FAM20A, MMP8 | | | | | | | | | |
| RSAD2, IFI27, OAS1, ISG15, OLAH, RETN, | 0.882 | 0.873 | 0.891 | 0.918 | 0.858 | 0.978 | 0.950 | 0.894 | 1 |
| MMP8 | | | | | | | | | |
| RSAD2, IFI27, IFIT1, SIGLEC1, HERC6, | 0.882 | 0.873 | 0.891 | 0.927 | 0.877 | 0.978 | 0.938 | 0.860 | 1 |
| SLC1A2, FAM20A | | | | | | | | | |
| RSAD2, IFI27, IFIT1, SIGLEC1, SLC1A2, | 0.882 | 0.873 | 0.891 | 0.924 | 0.869 | 0.980 | 0.941 | 0.865 | 1 |
| FAM20A, RETN | | | | | | | | | |
| IFI27, IFIT1, SIGLEC1, ISG15, HERC6, RETN, | 0.882 | 0.873 | 0.891 | 0.910 | 0.848 | 0.972 | 0.946 | 0.892 | 0.999 |
| MMP8 | | | | | | | | | |
| IFI27, IFIT1, HERC6, SLC1A2, IL1R2, OLAH, | 0.882 | 0.873 | 0.891 | 0.927 | 0.877 | 0.976 | 0.934 | 0.871 | 0.997 |
| MMP8 | | | | | | | | | |
| RSAD2, IFI27, IFI44L, HERC6, SLC1A2, OLAH, | 0.882 | 0.873 | 0.891 | 0.927 | 0.877 | 0.978 | 0.928 | 0.862 | 0.995 |
| MMP8 | | | | | | | | | |
| OAS1, IFIT1, SIGLEC1, ISG15, HERC6, | 0.882 | 0.873 | 0.891 | 0.916 | 0.858 | 0.975 | 0.941 | 0.875 | 1 |
| FAM20A, RETN | | | | | | | | | |
| SIGLEC1, ISG15, HERC6, IL1R2, FAM20A, | 0.882 | 0.873 | 0.891 | 0.915 | 0.855 | 0.975 | 0.961 | 0.900 | 1 |
| RETN, MMP8 | | | | | | | | | |
| RSAD2, IFI27, ISG15, IL1R2, OLAH, RETN, | 0.882 | 0.873 | 0.891 | 0.921 | 0.865 | 0.978 | 0.953 | 0.900 | 1 |
| MMP8 | | | | | | | | | |
| RSAD2, IFI27, IFIT1, ISG15, HERC6, SLC1A2, | 0.882 | 0.873 | 0.891 | 0.927 | 0.877 | 0.978 | 0.927 | 0.860 | 0.994 |
| OLAH | | | | | | | | | |
| RSAD2, IFI27, ISG15, HERC6, IL1R2, RETN, | 0.882 | 0.873 | 0.891 | 0.922 | 0.868 | 0.975 | 0.941 | 0.883 | 1.000 |
| MMP8 | | | | | | | | | |
| RSAD2, IFI27, IFI44L, SIGLEC1, SLC1A2, | 0.882 | 0.873 | 0.891 | 0.926 | 0.875 | 0.977 | 0.948 | 0.894 | 1 |
| OLAH, MMP8 | | | | | | | | | |
| IFI27, IFI44L, ISG15, HERC6, SLC1A2, OLAH, | 0.882 | 0.873 | 0.891 | 0.926 | 0.875 | 0.977 | 0.936 | 0.871 | 1 |
| RETN | | | | | | | | | |
| IFI27, IFI44L, ISG15, HERC6, SLC1A2, OLAH, | 0.882 | 0.873 | 0.891 | 0.928 | 0.878 | 0.978 | 0.926 | 0.859 | 0.993 |
| MMP8 | | | | | | | | | |
| RSAD2, IFI27, OAS1, IFI44L, OLAH, RETN, | 0.882 | 0.873 | 0.891 | 0.919 | 0.859 | 0.978 | 0.951 | 0.895 | 1 |
| MMP8 | | | | | | | | | |
| IFI27, OAS1, SIGLEC1, ISG15, IL1R2, RETN, | 0.882 | 0.873 | 0.891 | 0.923 | 0.871 | 0.976 | 0.963 | 0.918 | 1 |
| MMP8 | | | | | | | | | |
| IFI27, OAS1, IFI44L, SIGLEC1, ISG15, IL1R2, | 0.882 | 0.874 | 0.890 | 0.921 | 0.868 | 0.973 | 0.945 | 0.891 | 0.998 |
| RETN | | | | | | | | | |
| RSAD2, OAS1, IFIT1, SIGLEC1, HERC6, OLAH, | 0.882 | 0.873 | 0.891 | 0.912 | 0.851 | 0.973 | 0.949 | 0.893 | 1 |
| FAM20A | | | | | | | | | |
| RSAD2, IFI27, IFI44L, HERC6, SLC1A2, OLAH, | 0.882 | 0.873 | 0.891 | 0.926 | 0.875 | 0.977 | 0.935 | 0.870 | 0.999 |
| RETN | | | | | | | | | |
| RSAD2, IFI27, IFIT1, HERC6, SLC1A2, OLAH, | 0.882 | 0.873 | 0.891 | 0.927 | 0.876 | 0.978 | 0.930 | 0.865 | 0.995 |
| MMP8 | | | | | | | | | |
| IFI27, IFIT1, SIGLEC1, ISG15, IL1R2, RETN, | 0.882 | 0.873 | 0.891 | 0.921 | 0.867 | 0.975 | 0.964 | 0.925 | 1 |
| MMP8 | | | | | | | | | |
| IFI44L, SIGLEC1, IL1R2, OLAH, FAM20A, | 0.882 | 0.873 | 0.891 | 0.918 | 0.860 | 0.976 | 0.963 | 0.908 | 1 |
| RETN, MMP8 | | | | | | | | | |
| RSAD2, IFIT1, IFI44L, SIGLEC1, HERC6, | 0.882 | 0.873 | 0.891 | 0.915 | 0.855 | 0.975 | 0.943 | 0.884 | 1 |
| OLAH, FAM20A | | | | | | | | | |
| IFI27, IFIT1, IFI44L, SIGLEC1, SLC1A2, IL1R2, | 0.882 | 0.873 | 0.891 | 0.927 | 0.877 | 0.978 | 0.945 | 0.889 | 1.000 |
| OLAH | | | | | | | | | |
| IFI27, IFIT1, SLC1A2, IL1R2, OLAH, RETN, | 0.882 | 0.873 | 0.891 | 0.925 | 0.872 | 0.977 | 0.946 | 0.887 | 1 |
| MMP8 | | | | | | | | | |
| RSAD2, IFI27, IFI44L, SIGLEC1, ISG15, HERC6, | 0.882 | 0.873 | 0.891 | 0.917 | 0.859 | 0.975 | 0.943 | 0.889 | 0.998 |
| RETN | | | | | | | | | |
| IFIT1, IFI44L, SIGLEC1, ISG15, OLAH, | 0.882 | 0.872 | 0.892 | 0.909 | 0.844 | 0.974 | 0.954 | 0.904 | 1 |
| FAM20A, MMP8 | | | | | | | | | |
| RSAD2, IFI27, IFIT1, SIGLEC1, HERC6, IL1R2, | 0.882 | 0.874 | 0.890 | 0.922 | 0.875 | 0.969 | 0.947 | 0.898 | 0.995 |
| MMP8 | | | | | | | | | |
| IFI27, ISG15, HERC6, SLC1A2, IL1R2, OLAH, | 0.882 | 0.873 | 0.891 | 0.926 | 0.875 | 0.977 | 0.933 | 0.867 | 0.998 |
| RETN | | | | | | | | | |
| IFI27, ISG15, HERC6, SLC1A2, IL1R2, FAM20A, | 0.882 | 0.872 | 0.891 | 0.933 | 0.885 | 0.981 | 0.925 | 0.840 | 1 |
| RETN | | | | | | | | | |
| RSAD2, IFI27, OAS1, ISG15, IL1R2, OLAH, | 0.882 | 0.873 | 0.890 | 0.922 | 0.870 | 0.974 | 0.943 | 0.890 | 0.997 |
| MMP8 | | | | | | | | | |
| RSAD2, IFI27, OAS1, ISG15, SLC1A2, OLAH, | 0.882 | 0.873 | 0.891 | 0.926 | 0.873 | 0.979 | 0.929 | 0.861 | 0.998 |
| MMP8 | | | | | | | | | |
| IFI27, IFIT1, ISG15, SLC1A2, OLAH, RETN, | 0.882 | 0.872 | 0.891 | 0.927 | 0.873 | 0.980 | 0.940 | 0.878 | 1 |
| MMP8 | | | | | | | | | |
| RSAD2, IFI27, IFI44L, SIGLEC1, SLC1A2, | 0.882 | 0.873 | 0.890 | 0.927 | 0.875 | 0.978 | 0.951 | 0.895 | 1 |
| OLAH, RETN | | | | | | | | | |
| RSAD2, IFI27, IFIT1, HERC6, SLC1A2, IL1R2, | 0.882 | 0.873 | 0.891 | 0.926 | 0.876 | 0.975 | 0.930 | 0.866 | 0.995 |
| OLAH | | | | | | | | | |
| IFI27, HERC6, SLC1A2, IL1R2, OLAH, RETN, | 0.882 | 0.872 | 0.891 | 0.927 | 0.875 | 0.979 | 0.946 | 0.886 | 1 |
| MMP8 | | | | | | | | | |
| IFI27, OAS1, IFIT1, SLC1A2, OLAH, RETN, | 0.882 | 0.872 | 0.891 | 0.922 | 0.866 | 0.978 | 0.941 | 0.877 | 1 |
| MMP8 | | | | | | | | | |
| RSAD2, IFI27, IFIT1, SIGLEC1, IL1R2, RETN, | 0.882 | 0.873 | 0.890 | 0.922 | 0.868 | 0.975 | 0.962 | 0.921 | 1 |
| MMP8 | | | | | | | | | |
| RSAD2, IFI27, IFIT1, SIGLEC1, ISG15, | 0.882 | 0.872 | 0.891 | 0.916 | 0.852 | 0.981 | 0.940 | 0.868 | 1 |
| FAM20A, MMP8 | | | | | | | | | |
| RSAD2, IFI27, IFI44L, IL1R2, OLAH, RETN, | 0.882 | 0.873 | 0.891 | 0.923 | 0.868 | 0.979 | 0.951 | 0.897 | 1 |
| MMP8 | | | | | | | | | |
| IFI27, IFIT1, IFI44L, ISG15, HERC6, IL1R2, | 0.882 | 0.873 | 0.890 | 0.920 | 0.867 | 0.973 | 0.936 | 0.876 | 0.995 |
| RETN | | | | | | | | | |
| RSAD2, IFI27, IFIT1, IFI44L, OLAH, RETN, | 0.882 | 0.873 | 0.891 | 0.921 | 0.862 | 0.980 | 0.951 | 0.893 | 1 |
| MMP8 | | | | | | | | | |
| IFI27, OAS1, IFIT1, ISG15, IL1R2, OLAH, RETN | 0.882 | 0.873 | 0.890 | 0.918 | 0.862 | 0.975 | 0.941 | 0.882 | 1 |
| IFI44L, SIGLEC1, HERC6, SLC1A2, IL1R2, | 0.882 | 0.872 | 0.891 | 0.924 | 0.875 | 0.973 | 0.959 | 0.895 | 1 |
| FAM20A, MMP8 | | | | | | | | | |
| OAS1, IFIT1, SIGLEC1, HERC6, OLAH, | 0.882 | 0.873 | 0.891 | 0.915 | 0.855 | 0.975 | 0.946 | 0.886 | 1 |
| FAM20A, RETN | | | | | | | | | |
| IFI27, OAS1, IFI44L, ISG15, IL1R2, OLAH, | 0.882 | 0.873 | 0.890 | 0.923 | 0.872 | 0.974 | 0.934 | 0.874 | 0.993 |
| MMP8 | | | | | | | | | |
| IFIT1, SIGLEC1, ISG15, SLC1A2, IL1R2, | 0.882 | 0.873 | 0.891 | 0.927 | 0.881 | 0.973 | 0.959 | 0.895 | 1 |
| FAM20A, MMP8 | | | | | | | | | |
| OAS1, IFIT1, SIGLEC1, ISG15, HERC6, OLAH, | 0.882 | 0.872 | 0.891 | 0.913 | 0.853 | 0.973 | 0.949 | 0.893 | 1 |
| FAM20A | | | | | | | | | |
| IFI27, IFIT1, ISG15, HERC6, SLC1A2, OLAH, | 0.882 | 0.872 | 0.891 | 0.926 | 0.875 | 0.978 | 0.934 | 0.868 | 0.999 |
| RETN | | | | | | | | | |
| RSAD2, IFI27, IFIT1, ISG15, HERC6, IL1R2, | 0.882 | 0.873 | 0.890 | 0.920 | 0.868 | 0.973 | 0.943 | 0.889 | 0.998 |
| RETN | | | | | | | | | |
| RSAD2, IFI27, IFIT1, IFI44L, SIGLEC1, IL1R2, | 0.882 | 0.873 | 0.890 | 0.919 | 0.864 | 0.974 | 0.951 | 0.902 | 1 |
| RETN | | | | | | | | | |
| RSAD2, IFI27, OAS1, IFI44L, IL1R2, OLAH, | 0.882 | 0.873 | 0.890 | 0.922 | 0.869 | 0.974 | 0.945 | 0.892 | 0.998 |
| MMP8 | | | | | | | | | |
| OAS1, IFI44L, SIGLEC1, ISG15, IL1R2, | 0.882 | 0.872 | 0.891 | 0.917 | 0.859 | 0.975 | 0.961 | 0.902 | 1 |
| FAM20A, MMP8 | | | | | | | | | |
| IFI27, IFIT1, HERC6, SLC1A2, IL1R2, OLAH, | 0.882 | 0.872 | 0.891 | 0.925 | 0.874 | 0.977 | 0.936 | 0.871 | 1 |
| RETN | | | | | | | | | |
| IFI27, OAS1, SLC1A2, IL1R2, OLAH, RETN, | 0.882 | 0.872 | 0.891 | 0.925 | 0.873 | 0.977 | 0.942 | 0.880 | 1 |
| MMP8 | | | | | | | | | |
| RSAD2, IFI27, OAS1, SLC1A2, IL1R2, OLAH, | 0.882 | 0.873 | 0.890 | 0.926 | 0.876 | 0.976 | 0.930 | 0.862 | 0.999 |
| MMP8 | | | | | | | | | |
| IFI27, ISG15, HERC6, SLC1A2, IL1R2, FAM20A, | 0.882 | 0.872 | 0.891 | 0.930 | 0.884 | 0.977 | 0.925 | 0.839 | 1 |
| MMP8 | | | | | | | | | |
| IFI27, IFIT1, IFI44L, HERC6, SLC1A2, OLAH, | 0.882 | 0.872 | 0.891 | 0.924 | 0.871 | 0.976 | 0.935 | 0.869 | 1 |
| RETN | | | | | | | | | |
| RSAD2, IFI27, OAS1, IFIT1, HERC6, IL1R2, | 0.882 | 0.873 | 0.890 | 0.919 | 0.871 | 0.967 | 0.939 | 0.885 | 0.993 |
| MMP8 | | | | | | | | | |
| OAS1, IFIT1, IFI44L, SIGLEC1, IL1R2, OLAH, | 0.882 | 0.873 | 0.891 | 0.912 | 0.849 | 0.975 | 0.958 | 0.906 | 1 |
| FAM20A | | | | | | | | | |
| RSAD2, IFI27, SIGLEC1, ISG15, IL1R2, OLAH, | 0.882 | 0.873 | 0.890 | 0.919 | 0.863 | 0.975 | 0.948 | 0.893 | 1 |
| RETN | | | | | | | | | |
| IFI27, IFI44L, SIGLEC1, ISG15, SLC1A2, IL1R2, | 0.882 | 0.874 | 0.890 | 0.924 | 0.876 | 0.973 | 0.943 | 0.887 | 1.000 |
| RETN | | | | | | | | | |
| RSAD2, IFI27, IFI44L, SIGLEC1, SLC1A2, | 0.882 | 0.873 | 0.890 | 0.927 | 0.877 | 0.978 | 0.945 | 0.890 | 1.000 |
| IL1R2, OLAH | | | | | | | | | |
| IFI27, IFI44L, HERC6, SLC1A2, IL1R2, | 0.882 | 0.872 | 0.891 | 0.931 | 0.883 | 0.980 | 0.927 | 0.844 | 1 |
| FAM20A, RETN | | | | | | | | | |
| RSAD2, IFI27, IFIT1, SIGLEC1, SLC1A2, OLAH, | 0.882 | 0.873 | 0.890 | 0.926 | 0.874 | 0.978 | 0.949 | 0.892 | 1 |
| RETN | | | | | | | | | |
| RSAD2, IFI27, IFI44L, ISG15, HERC6, IL1R2, | 0.882 | 0.873 | 0.890 | 0.921 | 0.868 | 0.974 | 0.936 | 0.876 | 0.995 |
| RETN | | | | | | | | | |
| RSAD2, IFI27, OAS1, IFIT1, IFI44L, IL1R2, | 0.882 | 0.873 | 0.890 | 0.920 | 0.867 | 0.974 | 0.940 | 0.884 | 0.996 |
| OLAH | | | | | | | | | |
| OAS1, IFIT1, IFI44L, SIGLEC1, HERC6, | 0.882 | 0.873 | 0.890 | 0.915 | 0.855 | 0.975 | 0.937 | 0.872 | 1 |
| FAM20A, RETN | | | | | | | | | |
| IFI27, IFIT1, IFI44L, SLC1A2, IL1R2, FAM20A, | 0.882 | 0.872 | 0.891 | 0.926 | 0.876 | 0.977 | 0.924 | 0.837 | 1 |
| RETN | | | | | | | | | |
| IFI27, IFIT1, IFI44L, SIGLEC1, ISG15, IL1R2, | 0.882 | 0.874 | 0.889 | 0.922 | 0.874 | 0.970 | 0.942 | 0.892 | 0.993 |
| MMP8 | | | | | | | | | |
| RSAD2, IFI27, IFIT1, IFI44L, SIGLEC1, ISG15, | 0.882 | 0.873 | 0.890 | 0.919 | 0.863 | 0.976 | 0.941 | 0.885 | 0.997 |
| OLAH | | | | | | | | | |
| IFI27, IFI44L, ISG15, SLC1A2, IL1R2, OLAH, | 0.882 | 0.873 | 0.890 | 0.929 | 0.878 | 0.979 | 0.932 | 0.863 | 1 |
| RETN | | | | | | | | | |
| OAS1, IFIT1, SIGLEC1, SLC1A2, OLAH, | 0.882 | 0.872 | 0.891 | 0.922 | 0.869 | 0.976 | 0.952 | 0.892 | 1 |
| FAM20A, MMP8 | | | | | | | | | |
| IFI27, OAS1, IFIT1, IFI44L, HERC6, SLC1A2, | 0.882 | 0.872 | 0.891 | 0.929 | 0.879 | 0.978 | 0.934 | 0.868 | 0.999 |
| OLAH | | | | | | | | | |
| RSAD2, IFI27, OAS1, IFIT1, SIGLEC1, ISG15, | 0.882 | 0.873 | 0.890 | 0.919 | 0.863 | 0.975 | 0.941 | 0.885 | 0.998 |
| OLAH | | | | | | | | | |
| RSAD2, IFI27, IFI44L, SIGLEC1, ISG15, HERC6, | 0.881 | 0.874 | 0.889 | 0.920 | 0.870 | 0.970 | 0.940 | 0.888 | 0.992 |
| IL1R2 | | | | | | | | | |
| RSAD2, IFI27, OAS1, ISG15, IL1R2, OLAH, | 0.881 | 0.873 | 0.890 | 0.919 | 0.863 | 0.976 | 0.942 | 0.883 | 1 |
| RETN | | | | | | | | | |
| RSAD2, IFI27, IFIT1, ISG15, OLAH, RETN, | 0.881 | 0.872 | 0.891 | 0.919 | 0.859 | 0.978 | 0.950 | 0.893 | 1 |
| MMP8 | | | | | | | | | |
| RSAD2, IFI27, IFIT1, SIGLEC1, HERC6, RETN, | 0.881 | 0.872 | 0.890 | 0.915 | 0.856 | 0.974 | 0.943 | 0.890 | 0.997 |
| MMP8 | | | | | | | | | |
| RSAD2, IFI27, IFIT1, SIGLEC1, ISG15, OLAH, | 0.881 | 0.873 | 0.890 | 0.918 | 0.861 | 0.976 | 0.947 | 0.890 | 1 |
| RETN | | | | | | | | | |
| RSAD2, IFI27, IFI44L, ISG15, HERC6, SLC1A2, | 0.881 | 0.872 | 0.891 | 0.928 | 0.878 | 0.978 | 0.925 | 0.857 | 0.993 |
| OLAH | | | | | | | | | |
| RSAD2, IFI27, OAS1, IFIT1, SLC1A2, OLAH, | 0.881 | 0.873 | 0.890 | 0.926 | 0.875 | 0.978 | 0.940 | 0.877 | 1 |
| MMP8 | | | | | | | | | |
| RSAD2, IFI27, SLC1A2, IL1R2, OLAH, RETN, | 0.881 | 0.872 | 0.891 | 0.924 | 0.872 | 0.977 | 0.941 | 0.878 | 1 |
| MMP8 | | | | | | | | | |
| OAS1, IFIT1, SIGLEC1, ISG15, IL1R2, OLAH, | 0.881 | 0.872 | 0.890 | 0.913 | 0.850 | 0.976 | 0.961 | 0.910 | 1 |
| FAM20A | | | | | | | | | |
| IFI27, IFIT1, IFI44L, SIGLEC1, SLC1A2, | 0.881 | 0.872 | 0.890 | 0.920 | 0.863 | 0.978 | 0.942 | 0.868 | 1 |
| FAM20A, MMP8 | | | | | | | | | |
| RSAD2, IFI27, ISG15, SLC1A2, IL1R2, FAM20A, | 0.881 | 0.873 | 0.890 | 0.929 | 0.883 | 0.976 | 0.927 | 0.840 | 1 |
| MMP8 | | | | | | | | | |
| IFI27, OAS1, ISG15, SLC1A2, IL1R2, OLAH, | 0.881 | 0.873 | 0.890 | 0.927 | 0.877 | 0.977 | 0.922 | 0.852 | 0.992 |
| MMP8 | | | | | | | | | |
| IFI27, IFIT1, ISG15, SLC1A2, IL1R2, OLAH, | 0.881 | 0.873 | 0.890 | 0.927 | 0.877 | 0.978 | 0.936 | 0.870 | 1 |
| RETN | | | | | | | | | |
| IFI27, OAS1, IFIT1, IFI44L, IL1R2, OLAH, RETN | 0.881 | 0.873 | 0.890 | 0.916 | 0.860 | 0.973 | 0.942 | 0.883 | 1 |
| RSAD2, IFI27, OAS1, IFIT1, ISG15, IL1R2, | 0.881 | 0.873 | 0.890 | 0.919 | 0.865 | 0.972 | 0.941 | 0.885 | 0.998 |
| OLAH | | | | | | | | | |
| RSAD2, IFI27, IFIT1, SLC1A2, OLAH, RETN, | 0.881 | 0.872 | 0.891 | 0.923 | 0.868 | 0.979 | 0.945 | 0.884 | 1 |
| MMP8 | | | | | | | | | |
| OAS1, IFIT1, IFI44L, SIGLEC1, OLAH, | 0.881 | 0.871 | 0.891 | 0.910 | 0.844 | 0.975 | 0.957 | 0.907 | 1 |
| FAM20A, MMP8 | | | | | | | | | |
| RSAD2, IFI27, OAS1, ISG15, SLC1A2, IL1R2, | 0.881 | 0.872 | 0.890 | 0.930 | 0.880 | 0.979 | 0.932 | 0.864 | 0.999 |
| OLAH | | | | | | | | | |
| IFI27, IFI44L, SIGLEC1, HERC6, SLC1A2, | 0.881 | 0.872 | 0.891 | 0.925 | 0.870 | 0.980 | 0.945 | 0.866 | 1 |
| FAM20A, RETN | | | | | | | | | |
| SIGLEC1, HERC6, IL1R2, OLAH, FAM20A, | 0.881 | 0.871 | 0.891 | 0.918 | 0.859 | 0.976 | 0.965 | 0.911 | 1 |
| RETN, MMP8 | | | | | | | | | |
| RSAD2, IFI27, IFI44L, ISG15, IL1R2, OLAH, | 0.881 | 0.873 | 0.890 | 0.922 | 0.870 | 0.974 | 0.940 | 0.884 | 0.996 |
| MMP8 | | | | | | | | | |
| RSAD2, IFI27, OAS1, IFI44L, ISG15, IL1R2, | 0.881 | 0.873 | 0.890 | 0.922 | 0.869 | 0.975 | 0.942 | 0.887 | 0.997 |
| OLAH | | | | | | | | | |
| RSAD2, IFIT1, SIGLEC1, ISG15, HERC6, OLAH, | 0.881 | 0.872 | 0.891 | 0.912 | 0.852 | 0.973 | 0.951 | 0.896 | 1 |
| FAM20A | | | | | | | | | |
| IFI27, IFI44L, SIGLEC1, ISG15, SLC1A2, | 0.881 | 0.872 | 0.891 | 0.923 | 0.867 | 0.980 | 0.941 | 0.864 | 1 |
| FAM20A, RETN | | | | | | | | | |
| RSAD2, SIGLEC1, IL1R2, OLAH, FAM20A, | 0.881 | 0.872 | 0.891 | 0.919 | 0.862 | 0.976 | 0.964 | 0.913 | 1 |
| RETN, MMP8 | | | | | | | | | |
| IFI27, OAS1, IFIT1, SIGLEC1, ISG15, IL1R2, | 0.881 | 0.873 | 0.890 | 0.921 | 0.868 | 0.974 | 0.953 | 0.902 | 1 |
| RETN | | | | | | | | | |
| RSAD2, IFI27, IFIT1, SIGLEC1, ISG15, OLAH, | 0.881 | 0.873 | 0.890 | 0.919 | 0.863 | 0.975 | 0.941 | 0.885 | 0.998 |
| MMP8 | | | | | | | | | |
| RSAD2, IFI27, SIGLEC1, ISG15, HERC6, IL1R2, | 0.881 | 0.873 | 0.889 | 0.921 | 0.874 | 0.968 | 0.948 | 0.900 | 0.996 |
| MMP8 | | | | | | | | | |
| IFI27, OAS1, IFIT1, IFI44L, SIGLEC1, SLC1A2, | 0.881 | 0.872 | 0.890 | 0.922 | 0.864 | 0.979 | 0.940 | 0.867 | 1 |
| FAM20A | | | | | | | | | |
| IFI44L, SIGLEC1, SLC1A2, IL1R2, FAM20A, | 0.881 | 0.872 | 0.890 | 0.924 | 0.874 | 0.973 | 0.960 | 0.896 | 1 |
| RETN, MMP8 | | | | | | | | | |
| RSAD2, IFI27, OAS1, SIGLEC1, ISG15, OLAH, | 0.881 | 0.872 | 0.890 | 0.916 | 0.858 | 0.975 | 0.945 | 0.882 | 1 |
| RETN | | | | | | | | | |
| IFIT1, IFI44L, SIGLEC1, HERC6, SLC1A2, | 0.881 | 0.872 | 0.890 | 0.926 | 0.878 | 0.974 | 0.940 | 0.868 | 1 |
| IL1R2, FAM20A | | | | | | | | | |
| IFI27, OAS1, IFI44L, ISG15, HERC6, SLC1A2, | 0.881 | 0.871 | 0.891 | 0.929 | 0.880 | 0.979 | 0.924 | 0.854 | 0.994 |
| OLAH | | | | | | | | | |
| RSAD2, IFI27, OAS1, IFIT1, SIGLEC1, FAM20A, | 0.881 | 0.871 | 0.891 | 0.914 | 0.849 | 0.979 | 0.946 | 0.877 | 1 |
| MMP8 | | | | | | | | | |
| RSAD2, IFI27, OAS1, IFIT1, SLC1A2, IL1R2, | 0.881 | 0.872 | 0.890 | 0.925 | 0.874 | 0.976 | 0.936 | 0.869 | 1 |
| OLAH | | | | | | | | | |
| RSAD2, IFI27, OAS1, IFI44L, SIGLEC1, ISG15, | 0.881 | 0.872 | 0.891 | 0.915 | 0.851 | 0.979 | 0.943 | 0.871 | 1 |
| FAM20A | | | | | | | | | |
| RSAD2, IFI27, IFIT1, ISG15, SLC1A2, IL1R2, | 0.881 | 0.872 | 0.890 | 0.927 | 0.878 | 0.977 | 0.926 | 0.858 | 0.994 |
| OLAH | | | | | | | | | |
| IFI27, IFIT1, HERC6, SLC1A2, IL1R2, FAM20A, | 0.881 | 0.872 | 0.891 | 0.933 | 0.886 | 0.981 | 0.926 | 0.842 | 1 |
| RETN | | | | | | | | | |
| RSAD2, IFI27, OAS1, IFIT1, IL1R2, OLAH, | 0.881 | 0.872 | 0.890 | 0.917 | 0.860 | 0.974 | 0.945 | 0.886 | 1 |
| RETN | | | | | | | | | |
| RSAD2, IFI27, IFI44L, ISG15, SLC1A2, OLAH, | 0.881 | 0.872 | 0.890 | 0.927 | 0.875 | 0.979 | 0.928 | 0.860 | 0.996 |
| MMP8 | | | | | | | | | |
| IFI27, OAS1, IFIT1, IFI44L, SLC1A2, IL1R2, | 0.881 | 0.872 | 0.890 | 0.926 | 0.876 | 0.975 | 0.926 | 0.842 | 1 |
| FAM20A | | | | | | | | | |
| IFI27, OAS1, IFIT1, SLC1A2, IL1R2, OLAH, | 0.881 | 0.872 | 0.890 | 0.926 | 0.874 | 0.977 | 0.938 | 0.871 | 1 |
| RETN | | | | | | | | | |
| IFI27, OAS1, IFIT1, ISG15, SLC1A2, IL1R2, | 0.881 | 0.872 | 0.890 | 0.928 | 0.878 | 0.977 | 0.929 | 0.862 | 0.996 |
| OLAH | | | | | | | | | |
| RSAD2, IFI27, IFIT1, IFI44L, SIGLEC1, SLC1A2, | 0.881 | 0.873 | 0.890 | 0.926 | 0.874 | 0.977 | 0.945 | 0.889 | 1 |
| OLAH | | | | | | | | | |
| IFI27, IFIT1, SIGLEC1, ISG15, HERC6, IL1R2, | 0.881 | 0.873 | 0.889 | 0.921 | 0.873 | 0.969 | 0.950 | 0.903 | 0.997 |
| MMP8 | | | | | | | | | |
| IFI27, OAS1, IFIT1, IFI44L, ISG15, OLAH, | 0.881 | 0.872 | 0.890 | 0.916 | 0.858 | 0.974 | 0.935 | 0.875 | 0.994 |
| MMP8 | | | | | | | | | |
| RSAD2, IFIT1, SIGLEC1, HERC6, IL1R2, | 0.881 | 0.872 | 0.890 | 0.918 | 0.860 | 0.976 | 0.954 | 0.898 | 1 |
| FAM20A, RETN | | | | | | | | | |
| IFI27, IFI44L, SIGLEC1, ISG15, HERC6, | 0.881 | 0.871 | 0.891 | 0.911 | 0.848 | 0.974 | 0.941 | 0.862 | 1 |
| FAM20A, MMP8 | | | | | | | | | |
| IFI27, OAS1, SLC1A2, IL1R2, FAM20A, RETN, | 0.881 | 0.872 | 0.890 | 0.929 | 0.879 | 0.978 | 0.928 | 0.843 | 1 |
| MMP8 | | | | | | | | | |
| IFI27, IFIT1, IFI44L, ISG15, IL1R2, OLAH, | 0.881 | 0.872 | 0.889 | 0.917 | 0.861 | 0.974 | 0.939 | 0.879 | 1.000 |
| RETN | | | | | | | | | |
| RSAD2, IFI27, OAS1, SLC1A2, IL1R2, FAM20A, | 0.881 | 0.872 | 0.890 | 0.928 | 0.880 | 0.975 | 0.927 | 0.840 | 1 |
| MMP8 | | | | | | | | | |
| IFI27, OAS1, IFIT1, IFI44L, HERC6, RETN, | 0.881 | 0.872 | 0.890 | 0.915 | 0.854 | 0.976 | 0.935 | 0.866 | 1 |
| MMP8 | | | | | | | | | |
| RSAD2, IFI27, IFIT1, ISG15, IL1R2, OLAH, | 0.881 | 0.872 | 0.890 | 0.922 | 0.870 | 0.973 | 0.946 | 0.894 | 0.998 |
| MMP8 | | | | | | | | | |
| IFIT1, SIGLEC1, ISG15, HERC6, SLC1A2, | 0.881 | 0.871 | 0.891 | 0.921 | 0.868 | 0.974 | 0.937 | 0.864 | 1 |
| OLAH, FAM20A | | | | | | | | | |
| RSAD2, IFI44L, SIGLEC1, ISG15, IL1R2, | 0.881 | 0.872 | 0.890 | 0.916 | 0.858 | 0.975 | 0.961 | 0.903 | 1 |
| FAM20A, MMP8 | | | | | | | | | |
| IFI27, IFI44L, HERC6, SLC1A2, IL1R2, OLAH, | 0.881 | 0.871 | 0.890 | 0.924 | 0.872 | 0.976 | 0.936 | 0.871 | 1 |
| RETN | | | | | | | | | |
| RSAD2, IFI27, OAS1, SLC1A2, IL1R2, OLAH, | 0.881 | 0.872 | 0.890 | 0.926 | 0.874 | 0.978 | 0.937 | 0.867 | 1 |
| RETN | | | | | | | | | |
| IFI27, OAS1, IFI44L, SIGLEC1, SLC1A2, IL1R2, | 0.881 | 0.872 | 0.890 | 0.926 | 0.876 | 0.977 | 0.947 | 0.892 | 1 |
| OLAH | | | | | | | | | |
| RSAD2, IFI27, IFIT1, SIGLEC1, ISG15, HERC6, | 0.881 | 0.873 | 0.889 | 0.922 | 0.875 | 0.969 | 0.946 | 0.897 | 0.995 |
| IL1R2 | | | | | | | | | |
| RSAD2, OAS1, IFIT1, SIGLEC1, IL1R2, OLAH, | 0.881 | 0.872 | 0.890 | 0.912 | 0.849 | 0.975 | 0.965 | 0.917 | 1 |
| FAM20A | | | | | | | | | |
| RSAD2, OAS1, SIGLEC1, SLC1A2, IL1R2, | 0.881 | 0.871 | 0.890 | 0.922 | 0.874 | 0.971 | 0.960 | 0.896 | 1 |
| FAM20A, MMP8 | | | | | | | | | |
| IFI27, OAS1, IFIT1, SIGLEC1, SLC1A2, IL1R2, | 0.881 | 0.872 | 0.890 | 0.925 | 0.876 | 0.974 | 0.952 | 0.895 | 1 |
| RETN | | | | | | | | | |
| RSAD2, IFI27, OAS1, IFIT1, SIGLEC1, IL1R2, | 0.881 | 0.872 | 0.889 | 0.919 | 0.864 | 0.973 | 0.958 | 0.907 | 1 |
| RETN | | | | | | | | | |
| SIGLEC1, ISG15, HERC6, SLC1A2, IL1R2, | 0.881 | 0.871 | 0.890 | 0.924 | 0.876 | 0.972 | 0.959 | 0.893 | 1 |
| FAM20A, MMP8 | | | | | | | | | |
| IFIT1, SIGLEC1, HERC6, SLC1A2, IL1R2, | 0.881 | 0.871 | 0.891 | 0.924 | 0.874 | 0.974 | 0.943 | 0.872 | 1 |
| OLAH, FAM20A | | | | | | | | | |
| RSAD2, IFI27, OAS1, IFIT1, ISG15, OLAH, | 0.881 | 0.872 | 0.890 | 0.915 | 0.857 | 0.973 | 0.934 | 0.872 | 0.995 |
| MMP8 | | | | | | | | | |
| RSAD2, IFI27, SIGLEC1, ISG15, HERC6, RETN, | 0.881 | 0.871 | 0.890 | 0.918 | 0.860 | 0.976 | 0.954 | 0.907 | 1 |
| MMP8 | | | | | | | | | |
| RSAD2, IFI27, SIGLEC1, HERC6, SLC1A2, | 0.881 | 0.872 | 0.890 | 0.925 | 0.878 | 0.973 | 0.950 | 0.900 | 1 |
| IL1R2, RETN | | | | | | | | | |
| RSAD2, IFI27, OAS1, IFIT1, IFI44L, SIGLEC1, | 0.881 | 0.873 | 0.889 | 0.919 | 0.867 | 0.970 | 0.949 | 0.901 | 0.997 |
| IL1R2 | | | | | | | | | |
| RSAD2, IFI27, IFI44L, SIGLEC1, ISG15, IL1R2, | 0.881 | 0.872 | 0.889 | 0.920 | 0.865 | 0.974 | 0.947 | 0.896 | 0.998 |
| RETN | | | | | | | | | |
| RSAD2, IFI27, IFIT1, IFI44L, SLC1A2, IL1R2, | 0.881 | 0.872 | 0.890 | 0.924 | 0.874 | 0.975 | 0.927 | 0.843 | 1 |
| FAM20A | | | | | | | | | |
| IFI27, OAS1, IFIT1, IFI44L, ISG15, IL1R2, OLAH | 0.881 | 0.872 | 0.889 | 0.918 | 0.863 | 0.972 | 0.938 | 0.881 | 0.995 |
| RSAD2, SIGLEC1, ISG15, SLC1A2, IL1R2, | 0.881 | 0.871 | 0.890 | 0.926 | 0.878 | 0.973 | 0.959 | 0.893 | 1 |
| FAM20A, MMP8 | | | | | | | | | |
| OAS1, IFIT1, SIGLEC1, ISG15, OLAH, FAM20A, | 0.881 | 0.871 | 0.890 | 0.912 | 0.848 | 0.977 | 0.960 | 0.911 | 1 |
| MMP8 | | | | | | | | | |
| IFI27, OAS1, SIGLEC1, SLC1A2, IL1R2, RETN, | 0.881 | 0.872 | 0.889 | 0.927 | 0.880 | 0.974 | 0.963 | 0.916 | 1 |
| MMP8 | | | | | | | | | |
| IFI27, IFIT1, ISG15, HERC6, SLC1A2, IL1R2, | 0.881 | 0.871 | 0.890 | 0.929 | 0.882 | 0.977 | 0.925 | 0.840 | 1 |
| FAM20A | | | | | | | | | |
| IFI27, ISG15, SLC1A2, IL1R2, FAM20A, RETN, | 0.881 | 0.872 | 0.890 | 0.927 | 0.878 | 0.976 | 0.913 | 0.821 | 1 |
| MMP8 | | | | | | | | | |
| RSAD2, IFI27, IFIT1, IFI44L, SIGLEC1, SLC1A2, | 0.881 | 0.872 | 0.890 | 0.922 | 0.865 | 0.979 | 0.940 | 0.866 | 1 |
| FAM20A | | | | | | | | | |
| RSAD2, OAS1, IFIT1, SIGLEC1, OLAH, | 0.881 | 0.871 | 0.890 | 0.912 | 0.848 | 0.977 | 0.959 | 0.910 | 1 |
| FAM20A, MMP8 | | | | | | | | | |
| RSAD2, IFI27, OAS1, IFI44L, HERC6, SLC1A2, | 0.881 | 0.871 | 0.890 | 0.929 | 0.879 | 0.978 | 0.932 | 0.864 | 0.999 |
| OLAH | | | | | | | | | |
| RSAD2, IFI27, IFI44L, SIGLEC1, HERC6, RETN, | 0.881 | 0.872 | 0.890 | 0.916 | 0.857 | 0.974 | 0.943 | 0.889 | 0.998 |
| MMP8 | | | | | | | | | |
| RSAD2, IFI27, OAS1, IFIT1, ISG15, SLC1A2, | 0.881 | 0.872 | 0.890 | 0.926 | 0.873 | 0.978 | 0.933 | 0.866 | 0.999 |
| OLAH | | | | | | | | | |
| IFI27, OAS1, IFIT1, IFI44L, HERC6, IL1R2, | 0.881 | 0.873 | 0.889 | 0.921 | 0.872 | 0.970 | 0.938 | 0.883 | 0.993 |
| MMP8 | | | | | | | | | |
| RSAD2, IFI27, OAS1, IFI44L, HERC6, IL1R2, | 0.881 | 0.873 | 0.889 | 0.919 | 0.871 | 0.968 | 0.935 | 0.877 | 0.992 |
| MMP8 | | | | | | | | | |
| IFI27, IFI44L, ISG15, HERC6, SLC1A2, IL1R2, | 0.881 | 0.872 | 0.890 | 0.928 | 0.879 | 0.977 | 0.926 | 0.858 | 0.994 |
| OLAH | | | | | | | | | |
| IFI27, IFIT1, IFI44L, ISG15, SLC1A2, IL1R2, | 0.881 | 0.871 | 0.890 | 0.925 | 0.876 | 0.975 | 0.927 | 0.842 | 1 |
| FAM20A | | | | | | | | | |
| RSAD2, IFI27, OAS1, IFI44L, ISG15, SLC1A2, | 0.881 | 0.872 | 0.890 | 0.926 | 0.873 | 0.978 | 0.929 | 0.861 | 0.998 |
| OLAH | | | | | | | | | |
| RSAD2, IFI27, OAS1, IFI44L, ISG15, OLAH, | 0.881 | 0.872 | 0.889 | 0.917 | 0.860 | 0.975 | 0.935 | 0.876 | 0.994 |
| MMP8 | | | | | | | | | |
| OAS1, SIGLEC1, IL1R2, OLAH, FAM20A, | 0.881 | 0.871 | 0.890 | 0.918 | 0.860 | 0.976 | 0.965 | 0.913 | 1 |
| RETN, MMP8 | | | | | | | | | |
| OAS1, IFIT1, SIGLEC1, HERC6, SLC1A2, | 0.881 | 0.871 | 0.890 | 0.925 | 0.877 | 0.973 | 0.945 | 0.873 | 1 |
| IL1R2, FAM20A | | | | | | | | | |
| IFIT1, SIGLEC1, ISG15, HERC6, SLC1A2, | 0.881 | 0.871 | 0.890 | 0.924 | 0.875 | 0.973 | 0.946 | 0.874 | 1 |
| IL1R2, FAM20A | | | | | | | | | |
| OAS1, SIGLEC1, ISG15, IL1R2, FAM20A, | 0.881 | 0.871 | 0.890 | 0.917 | 0.859 | 0.976 | 0.964 | 0.910 | 1 |
| RETN, MMP8 | | | | | | | | | |
| RSAD2, IFI44L, SIGLEC1, SLC1A2, IL1R2, | 0.881 | 0.871 | 0.890 | 0.923 | 0.874 | 0.972 | 0.959 | 0.895 | 1 |
| FAM20A, MMP8 | | | | | | | | | |
| IFI27, OAS1, IFIT1, ISG15, SLC1A2, OLAH, | 0.881 | 0.872 | 0.890 | 0.926 | 0.874 | 0.978 | 0.938 | 0.872 | 1 |
| RETN | | | | | | | | | |
| OAS1, IFI44L, SIGLEC1, IL1R2, FAM20A, | 0.881 | 0.871 | 0.890 | 0.918 | 0.860 | 0.976 | 0.963 | 0.908 | 1 |
| RETN, MMP8 | | | | | | | | | |
| RSAD2, IFI27, SIGLEC1, ISG15, IL1R2, RETN, | 0.881 | 0.872 | 0.889 | 0.922 | 0.869 | 0.975 | 0.963 | 0.922 | 1 |
| MMP8 | | | | | | | | | |
| IFIT1, SIGLEC1, SLC1A2, IL1R2, FAM20A, | 0.881 | 0.871 | 0.890 | 0.928 | 0.881 | 0.975 | 0.959 | 0.893 | 1 |
| RETN, MMP8 | | | | | | | | | |
| IFI27, OAS1, ISG15, SLC1A2, OLAH, RETN, | 0.881 | 0.871 | 0.890 | 0.927 | 0.874 | 0.979 | 0.937 | 0.871 | 1 |
| MMP8 | | | | | | | | | |
| IFI27, OAS1, IFI44L, ISG15, IL1R2, OLAH, | 0.880 | 0.872 | 0.889 | 0.919 | 0.863 | 0.975 | 0.940 | 0.881 | 0.999 |
| RETN | | | | | | | | | |
| RSAD2, IFI27, OAS1, SLC1A2, OLAH, RETN, | 0.880 | 0.871 | 0.890 | 0.925 | 0.871 | 0.980 | 0.940 | 0.875 | 1 |
| MMP8 | | | | | | | | | |
| IFI27, IFI44L, SIGLEC1, HERC6, SLC1A2, | 0.880 | 0.872 | 0.889 | 0.926 | 0.876 | 0.976 | 0.946 | 0.894 | 0.998 |
| IL1R2, RETN | | | | | | | | | |
| IFI27, OAS1, IFIT1, SLC1A2, IL1R2, FAM20A, | 0.880 | 0.871 | 0.890 | 0.927 | 0.877 | 0.978 | 0.924 | 0.839 | 1 |
| RETN | | | | | | | | | |
| IFI27, IFI44L, SIGLEC1, SLC1A2, IL1R2, RETN, | 0.880 | 0.872 | 0.889 | 0.929 | 0.882 | 0.975 | 0.963 | 0.922 | 1 |
| MMP8 | | | | | | | | | |
| RSAD2, IFIT1, SIGLEC1, HERC6, FAM20A, | 0.880 | 0.871 | 0.889 | 0.917 | 0.857 | 0.977 | 0.940 | 0.872 | 1 |
| RETN, MMP8 | | | | | | | | | |
| RSAD2, OAS1, IFIT1, SIGLEC1, HERC6, | 0.880 | 0.871 | 0.890 | 0.916 | 0.856 | 0.975 | 0.942 | 0.876 | 1 |
| FAM20A, RETN | | | | | | | | | |
| IFI27, OAS1, IFI44L, ISG15, SLC1A2, OLAH, | 0.880 | 0.871 | 0.890 | 0.927 | 0.875 | 0.979 | 0.924 | 0.853 | 0.995 |
| MMP8 | | | | | | | | | |
| RSAD2, IFI27, ISG15, SLC1A2, OLAH, RETN, | 0.880 | 0.871 | 0.890 | 0.927 | 0.873 | 0.980 | 0.938 | 0.873 | 1 |
| MMP8 | | | | | | | | | |
| RSAD2, IFI27, OAS1, IFIT1, IFI44L, OLAH, | 0.880 | 0.872 | 0.889 | 0.916 | 0.858 | 0.974 | 0.941 | 0.884 | 0.999 |
| MMP8 | | | | | | | | | |
| RSAD2, IFI27, IFI44L, SLC1A2, OLAH, RETN, | 0.880 | 0.871 | 0.890 | 0.926 | 0.872 | 0.980 | 0.942 | 0.880 | 1 |
| MMP8 | | | | | | | | | |
| IFI27, IFIT1, IFI44L, HERC6, SLC1A2, OLAH, | 0.880 | 0.871 | 0.890 | 0.927 | 0.877 | 0.978 | 0.932 | 0.868 | 0.995 |
| MMP8 | | | | | | | | | |
| RSAD2, IFI27, IFIT1, SLC1A2, IL1R2, OLAH, | 0.880 | 0.872 | 0.889 | 0.923 | 0.871 | 0.976 | 0.934 | 0.866 | 1 |
| RETN | | | | | | | | | |
| IFI27, OAS1, IFI44L, SIGLEC1, SLC1A2, | 0.880 | 0.871 | 0.890 | 0.922 | 0.865 | 0.979 | 0.938 | 0.860 | 1 |
| FAM20A, RETN | | | | | | | | | |
| RSAD2, IFIT1, SIGLEC1, SLC1A2, OLAH, | 0.880 | 0.871 | 0.890 | 0.923 | 0.870 | 0.976 | 0.951 | 0.892 | 1 |
| FAM20A, MMP8 | | | | | | | | | |
| RSAD2, IFI27, IFI44L, ISG15, SLC1A2, IL1R2, | 0.880 | 0.871 | 0.890 | 0.929 | 0.882 | 0.977 | 0.927 | 0.843 | 1 |
| FAM20A | | | | | | | | | |
| IFI27, IFIT1, IFI44L, SLC1A2, IL1R2, OLAH, | 0.880 | 0.872 | 0.889 | 0.925 | 0.875 | 0.975 | 0.937 | 0.877 | 0.997 |
| MMP8 | | | | | | | | | |
| RSAD2, SIGLEC1, ISG15, IL1R2, FAM20A, | 0.880 | 0.871 | 0.890 | 0.918 | 0.861 | 0.976 | 0.963 | 0.909 | 1 |
| RETN, MMP8 | | | | | | | | | |
| RSAD2, IFI27, OAS1, IFIT1, HERC6, RETN, | 0.880 | 0.871 | 0.890 | 0.915 | 0.853 | 0.977 | 0.934 | 0.862 | 1 |
| MMP8 | | | | | | | | | |
| IFIT1, IFI44L, SIGLEC1, ISG15, IL1R2, OLAH, | 0.880 | 0.872 | 0.889 | 0.909 | 0.845 | 0.973 | 0.958 | 0.907 | 1 |
| FAM20A | | | | | | | | | |
| IFI27, OAS1, IFIT1, IFI44L, SLC1A2, OLAH, | 0.880 | 0.871 | 0.890 | 0.927 | 0.875 | 0.978 | 0.939 | 0.877 | 1 |
| MMP8 | | | | | | | | | |
| IFI44L, SIGLEC1, ISG15, HERC6, IL1R2, OLAH, | 0.880 | 0.871 | 0.890 | 0.914 | 0.852 | 0.975 | 0.957 | 0.898 | 1 |
| FAM20A | | | | | | | | | |
| IFI27, IFIT1, IFI44L, ISG15, HERC6, SLC1A2, | 0.880 | 0.871 | 0.889 | 0.928 | 0.877 | 0.978 | 0.929 | 0.865 | 0.994 |
| OLAH | | | | | | | | | |
| RSAD2, IFI27, OAS1, IFIT1, IFI44L, HERC6, | 0.880 | 0.872 | 0.888 | 0.919 | 0.870 | 0.969 | 0.937 | 0.881 | 0.993 |
| IL1R2 | | | | | | | | | |
| IFI27, OAS1, IFIT1, SIGLEC1, HERC6, SLC1A2, | 0.880 | 0.872 | 0.889 | 0.926 | 0.881 | 0.971 | 0.950 | 0.900 | 1.000 |
| IL1R2 | | | | | | | | | |
| IFI27, IFI44L, HERC6, SLC1A2, IL1R2, OLAH, | 0.880 | 0.871 | 0.890 | 0.927 | 0.877 | 0.976 | 0.930 | 0.866 | 0.995 |
| MMP8 | | | | | | | | | |
| RSAD2, IFI27, OAS1, IFI44L, SIGLEC1, IL1R2, | 0.880 | 0.872 | 0.889 | 0.920 | 0.870 | 0.970 | 0.952 | 0.907 | 0.997 |
| MMP8 | | | | | | | | | |
| IFI27, OAS1, ISG15, HERC6, SLC1A2, FAM20A, | 0.880 | 0.870 | 0.890 | 0.922 | 0.867 | 0.978 | 0.930 | 0.849 | 1 |
| RETN | | | | | | | | | |
| RSAD2, IFI27, IFIT1, IFI44L, SIGLEC1, IL1R2, | 0.880 | 0.872 | 0.888 | 0.920 | 0.871 | 0.970 | 0.946 | 0.897 | 0.994 |
| MMP8 | | | | | | | | | |
| OAS1, IFIT1, SIGLEC1, IL1R2, OLAH, FAM20A, | 0.880 | 0.871 | 0.889 | 0.911 | 0.848 | 0.974 | 0.962 | 0.911 | 1 |
| RETN | | | | | | | | | |
| RSAD2, IFI27, IFI44L, ISG15, OLAH, RETN, | 0.880 | 0.871 | 0.889 | 0.918 | 0.859 | 0.978 | 0.948 | 0.890 | 1 |
| MMP8 | | | | | | | | | |
| SIGLEC1, ISG15, SLC1A2, IL1R2, FAM20A, | 0.880 | 0.871 | 0.889 | 0.924 | 0.875 | 0.973 | 0.955 | 0.885 | 1 |
| RETN, MMP8 | | | | | | | | | |
| OAS1, IFIT1, IFI44L, SIGLEC1, ISG15, IL1R2, | 0.880 | 0.871 | 0.889 | 0.912 | 0.849 | 0.975 | 0.959 | 0.907 | 1 |
| FAM20A | | | | | | | | | |
| IFI27, IFIT1, SIGLEC1, HERC6, SLC1A2, IL1R2, | 0.880 | 0.871 | 0.889 | 0.927 | 0.881 | 0.974 | 0.952 | 0.905 | 1.000 |
| RETN | | | | | | | | | |
| IFI27, IFIT1, IFI44L, SLC1A2, OLAH, RETN, | 0.880 | 0.871 | 0.890 | 0.923 | 0.867 | 0.979 | 0.943 | 0.882 | 1 |
| MMP8 | | | | | | | | | |
| RSAD2, IFI27, OAS1, HERC6, SLC1A2, IL1R2, | 0.880 | 0.871 | 0.889 | 0.924 | 0.874 | 0.974 | 0.932 | 0.858 | 1 |
| RETN | | | | | | | | | |
| RSAD2, IFI27, OAS1, IFIT1, ISG15, OLAH, | 0.880 | 0.871 | 0.889 | 0.915 | 0.855 | 0.974 | 0.941 | 0.879 | 1 |
| RETN | | | | | | | | | |
| IFI27, OAS1, IFIT1, IFI44L, SIGLEC1, ISG15, | 0.880 | 0.872 | 0.888 | 0.920 | 0.871 | 0.969 | 0.946 | 0.896 | 0.995 |
| IL1R2 | | | | | | | | | |
| RSAD2, IFI27, OAS1, IFIT1, IFI44L, OLAH, | 0.880 | 0.871 | 0.889 | 0.915 | 0.855 | 0.974 | 0.946 | 0.884 | 1 |
| RETN | | | | | | | | | |
| IFI27, OAS1, IFIT1, IFI44L, ISG15, OLAH, RETN | 0.880 | 0.871 | 0.889 | 0.916 | 0.857 | 0.975 | 0.940 | 0.878 | 1 |
| IFI27, IFIT1, IFI44L, ISG15, SLC1A2, IL1R2, | 0.880 | 0.871 | 0.889 | 0.927 | 0.878 | 0.977 | 0.927 | 0.860 | 0.994 |
| OLAH | | | | | | | | | |
| RSAD2, IFI27, ISG15, SLC1A2, IL1R2, OLAH, | 0.880 | 0.871 | 0.889 | 0.926 | 0.875 | 0.978 | 0.932 | 0.863 | 1.000 |
| RETN | | | | | | | | | |
| RSAD2, SIGLEC1, SLC1A2, IL1R2, FAM20A, | 0.880 | 0.871 | 0.890 | 0.924 | 0.875 | 0.973 | 0.958 | 0.894 | 1 |
| RETN, MMP8 | | | | | | | | | |
| RSAD2, IFI27, OAS1, SIGLEC1, HERC6, | 0.880 | 0.871 | 0.889 | 0.927 | 0.882 | 0.972 | 0.953 | 0.905 | 1 |
| SLC1A2, IL1R2 | | | | | | | | | |
| IFI27, SIGLEC1, HERC6, SLC1A2, IL1R2, | 0.880 | 0.871 | 0.889 | 0.925 | 0.872 | 0.977 | 0.946 | 0.889 | 1 |
| OLAH, RETN | | | | | | | | | |
| IFI27, OAS1, SIGLEC1, HERC6, SLC1A2, IL1R2, | 0.880 | 0.871 | 0.889 | 0.927 | 0.882 | 0.971 | 0.948 | 0.897 | 0.999 |
| MMP8 | | | | | | | | | |
| RSAD2, IFI27, OAS1, SIGLEC1, SLC1A2, | 0.880 | 0.870 | 0.890 | 0.923 | 0.866 | 0.980 | 0.939 | 0.862 | 1 |
| FAM20A, RETN | | | | | | | | | |
| OAS1, IFI44L, SIGLEC1, SLC1A2, IL1R2, | 0.880 | 0.871 | 0.889 | 0.923 | 0.874 | 0.971 | 0.961 | 0.901 | 1 |
| FAM20A, MMP8 | | | | | | | | | |
| RSAD2, IFIT1, SIGLEC1, ISG15, HERC6, | 0.880 | 0.871 | 0.889 | 0.916 | 0.857 | 0.976 | 0.942 | 0.875 | 1 |
| FAM20A, RETN | | | | | | | | | |
| RSAD2, IFI27, OAS1, IFI44L, HERC6, RETN, | 0.880 | 0.871 | 0.889 | 0.915 | 0.853 | 0.977 | 0.934 | 0.862 | 1 |
| MMP8 | | | | | | | | | |
| RSAD2, IFI27, IFI44L, SLC1A2, IL1R2, OLAH, | 0.880 | 0.871 | 0.889 | 0.925 | 0.875 | 0.976 | 0.935 | 0.871 | 0.998 |
| MMP8 | | | | | | | | | |
| IFI27, IFIT1, IFI44L, ISG15, SLC1A2, OLAH, | 0.880 | 0.871 | 0.889 | 0.927 | 0.876 | 0.979 | 0.934 | 0.866 | 1 |
| RETN | | | | | | | | | |
| RSAD2, IFI27, OAS1, IFI44L, SLC1A2, IL1R2, | 0.880 | 0.871 | 0.889 | 0.927 | 0.879 | 0.975 | 0.927 | 0.843 | 1 |
| FAM20A | | | | | | | | | |
| IFI27, OAS1, IFI44L, ISG15, SLC1A2, IL1R2, | 0.880 | 0.871 | 0.889 | 0.930 | 0.882 | 0.977 | 0.926 | 0.842 | 1 |
| FAM20A | | | | | | | | | |
| RSAD2, IFI27, OAS1, SIGLEC1, ISG15, IL1R2, | 0.880 | 0.871 | 0.889 | 0.920 | 0.866 | 0.973 | 0.955 | 0.902 | 1 |
| RETN | | | | | | | | | |
| RSAD2, IFI27, IFI44L, ISG15, IL1R2, OLAH, | 0.880 | 0.871 | 0.889 | 0.918 | 0.860 | 0.975 | 0.939 | 0.878 | 1 |
| RETN | | | | | | | | | |
| RSAD2, IFIT1, SIGLEC1, HERC6, SLC1A2, | 0.880 | 0.871 | 0.889 | 0.925 | 0.877 | 0.974 | 0.946 | 0.874 | 1 |
| IL1R2, FAM20A | | | | | | | | | |
| IFI27, IFIT1, IFI44L, SIGLEC1, SLC1A2, IL1R2, | 0.880 | 0.871 | 0.888 | 0.923 | 0.872 | 0.973 | 0.950 | 0.897 | 1 |
| RETN | | | | | | | | | |
| RSAD2, IFIT1, SIGLEC1, HERC6, SLC1A2, | 0.880 | 0.870 | 0.890 | 0.921 | 0.868 | 0.974 | 0.939 | 0.866 | 1 |
| OLAH, FAM20A | | | | | | | | | |
| RSAD2, IFIT1, IFI44L, SIGLEC1, IL1R2, OLAH, | 0.880 | 0.871 | 0.889 | 0.911 | 0.847 | 0.974 | 0.962 | 0.913 | 1 |
| FAM20A | | | | | | | | | |
| IFI27, IFIT1, ISG15, HERC6, IL1R2, RETN, | 0.880 | 0.871 | 0.889 | 0.922 | 0.868 | 0.975 | 0.946 | 0.892 | 0.999 |
| MMP8 | | | | | | | | | |
| IFI27, IFI44L, SIGLEC1, SLC1A2, FAM20A, | 0.880 | 0.870 | 0.890 | 0.923 | 0.867 | 0.980 | 0.950 | 0.875 | 1 |
| RETN, MMP8 | | | | | | | | | |
| IFI27, OAS1, ISG15, SLC1A2, IL1R2, FAM20A, | 0.880 | 0.871 | 0.889 | 0.927 | 0.878 | 0.975 | 0.927 | 0.841 | 1 |
| MMP8 | | | | | | | | | |
| RSAD2, IFI27, IFI44L, ISG15, SLC1A2, IL1R2, | 0.880 | 0.871 | 0.889 | 0.927 | 0.877 | 0.977 | 0.929 | 0.863 | 0.996 |
| OLAH | | | | | | | | | |
| RSAD2, IFI27, IFIT1, ISG15, IL1R2, OLAH, | 0.880 | 0.871 | 0.888 | 0.919 | 0.863 | 0.975 | 0.941 | 0.882 | 1 |
| RETN | | | | | | | | | |
| RSAD2, IFI27, OAS1, IFIT1, IL1R2, RETN, | 0.880 | 0.871 | 0.889 | 0.920 | 0.865 | 0.974 | 0.953 | 0.902 | 1 |
| MMP8 | | | | | | | | | |
| RSAD2, IFI27, IFIT1, IFI44L, ISG15, IL1R2, | 0.880 | 0.872 | 0.888 | 0.919 | 0.865 | 0.973 | 0.938 | 0.881 | 0.995 |
| OLAH | | | | | | | | | |
| IFI27, OAS1, IFIT1, HERC6, SLC1A2, IL1R2, | 0.880 | 0.871 | 0.889 | 0.924 | 0.875 | 0.973 | 0.938 | 0.873 | 1 |
| RETN | | | | | | | | | |
| IFI27, OAS1, IFI44L, HERC6, SLC1A2, | 0.880 | 0.870 | 0.889 | 0.923 | 0.868 | 0.979 | 0.935 | 0.853 | 1 |
| FAM20A, RETN | | | | | | | | | |
| RSAD2, IFI27, OAS1, IFI44L, IL1R2, OLAH, | 0.880 | 0.871 | 0.889 | 0.918 | 0.862 | 0.975 | 0.945 | 0.886 | 1 |
| RETN | | | | | | | | | |
| IFI27, OAS1, ISG15, SLC1A2, IL1R2, OLAH, | 0.880 | 0.871 | 0.889 | 0.927 | 0.876 | 0.977 | 0.930 | 0.861 | 1.000 |
| RETN | | | | | | | | | |
| RSAD2, IFIT1, SIGLEC1, HERC6, OLAH, | 0.880 | 0.871 | 0.889 | 0.915 | 0.854 | 0.976 | 0.948 | 0.889 | 1 |
| FAM20A, RETN | | | | | | | | | |
| IFI27, OAS1, IFI44L, HERC6, SLC1A2, IL1R2, | 0.880 | 0.870 | 0.890 | 0.928 | 0.879 | 0.977 | 0.928 | 0.860 | 0.997 |
| OLAH | | | | | | | | | |
| IFI27, OAS1, IFIT1, ISG15, SLC1A2, IL1R2, | 0.880 | 0.870 | 0.889 | 0.924 | 0.874 | 0.974 | 0.925 | 0.840 | 1 |
| FAM20A | | | | | | | | | |
| RSAD2, IFI27, IFIT1, IFI44L, HERC6, SLC1A2, | 0.880 | 0.871 | 0.889 | 0.927 | 0.877 | 0.978 | 0.930 | 0.865 | 0.995 |
| OLAH | | | | | | | | | |
| OAS1, IFIT1, SIGLEC1, HERC6, SLC1A2, | 0.880 | 0.870 | 0.890 | 0.922 | 0.870 | 0.973 | 0.937 | 0.867 | 1 |
| OLAH, FAM20A | | | | | | | | | |
| RSAD2, IFI27, SIGLEC1, ISG15, SLC1A2, | 0.880 | 0.870 | 0.890 | 0.922 | 0.866 | 0.979 | 0.941 | 0.866 | 1 |
| FAM20A, RETN | | | | | | | | | |
| RSAD2, IFI27, OAS1, IFI44L, SLC1A2, OLAH, | 0.880 | 0.870 | 0.889 | 0.924 | 0.872 | 0.977 | 0.937 | 0.872 | 1 |
| MMP8 | | | | | | | | | |
| IFI27, IFIT1, SIGLEC1, ISG15, SLC1A2, | 0.880 | 0.870 | 0.889 | 0.922 | 0.865 | 0.978 | 0.941 | 0.865 | 1 |
| FAM20A, MMP8 | | | | | | | | | |
| RSAD2, IFI27, SIGLEC1, SLC1A2, IL1R2, | 0.880 | 0.871 | 0.888 | 0.928 | 0.881 | 0.975 | 0.964 | 0.922 | 1 |
| RETN, MMP8 | | | | | | | | | |
| RSAD2, IFI27, OAS1, IFIT1, SLC1A2, OLAH, | 0.880 | 0.871 | 0.889 | 0.927 | 0.874 | 0.979 | 0.940 | 0.873 | 1 |
| RETN | | | | | | | | | |
| RSAD2, IFI27, IFI44L, HERC6, SLC1A2, IL1R2, | 0.880 | 0.870 | 0.889 | 0.926 | 0.876 | 0.976 | 0.929 | 0.864 | 0.995 |
| OLAH | | | | | | | | | |
| IFI27, IFIT1, SIGLEC1, SLC1A2, IL1R2, RETN, | 0.880 | 0.871 | 0.888 | 0.926 | 0.878 | 0.973 | 0.967 | 0.929 | 1 |
| MMP8 | | | | | | | | | |
| IFI27, OAS1, HERC6, SLC1A2, IL1R2, RETN, | 0.880 | 0.871 | 0.888 | 0.925 | 0.876 | 0.974 | 0.926 | 0.854 | 0.998 |
| MMP8 | | | | | | | | | |
| IFIT1, IFI44L, SIGLEC1, ISG15, SLC1A2, OLAH, | 0.880 | 0.870 | 0.889 | 0.920 | 0.865 | 0.975 | 0.934 | 0.868 | 1.000 |
| FAM20A | | | | | | | | | |
| IFI27, IFIT1, SIGLEC1, ISG15, SLC1A2, IL1R2, | 0.880 | 0.871 | 0.888 | 0.925 | 0.877 | 0.973 | 0.952 | 0.901 | 1 |
| RETN | | | | | | | | | |
| IFI27, OAS1, IFI44L, SLC1A2, IL1R2, FAM20A, | 0.880 | 0.870 | 0.889 | 0.926 | 0.875 | 0.977 | 0.925 | 0.840 | 1 |
| RETN | | | | | | | | | |
| IFI27, OAS1, IFI44L, SLC1A2, OLAH, RETN, | 0.879 | 0.870 | 0.889 | 0.926 | 0.872 | 0.979 | 0.942 | 0.880 | 1 |
| MMP8 | | | | | | | | | |
| IFI27, OAS1, IFI44L, SIGLEC1, ISG15, IL1R2, | 0.879 | 0.872 | 0.887 | 0.919 | 0.870 | 0.968 | 0.940 | 0.888 | 0.992 |
| MMP8 | | | | | | | | | |
| IFI27, IFIT1, IFI44L, SLC1A2, IL1R2, OLAH, | 0.879 | 0.871 | 0.888 | 0.922 | 0.870 | 0.975 | 0.935 | 0.869 | 1 |
| RETN | | | | | | | | | |
| IFIT1, IFI44L, SIGLEC1, ISG15, HERC6, | 0.879 | 0.870 | 0.889 | 0.913 | 0.851 | 0.975 | 0.935 | 0.863 | 1 |
| FAM20A, MMP8 | | | | | | | | | |
| IFI27, IFI44L, SIGLEC1, ISG15, SLC1A2, IL1R2, | 0.879 | 0.872 | 0.887 | 0.924 | 0.879 | 0.970 | 0.945 | 0.893 | 0.996 |
| MMP8 | | | | | | | | | |
| RSAD2, IFI27, IFIT1, IFI44L, IL1R2, OLAH, | 0.879 | 0.871 | 0.888 | 0.916 | 0.859 | 0.974 | 0.942 | 0.883 | 1 |
| RETN | | | | | | | | | |
| IFI27, OAS1, IFIT1, IFI44L, IL1R2, RETN, | 0.879 | 0.871 | 0.888 | 0.919 | 0.864 | 0.975 | 0.945 | 0.889 | 1 |
| MMP8 | | | | | | | | | |
| RSAD2, IFI27, IFIT1, IFI44L, SLC1A2, OLAH, | 0.879 | 0.871 | 0.888 | 0.926 | 0.875 | 0.978 | 0.935 | 0.871 | 0.998 |
| MMP8 | | | | | | | | | |
| RSAD2, IFI27, OAS1, SIGLEC1, SLC1A2, IL1R2, | 0.879 | 0.871 | 0.888 | 0.922 | 0.872 | 0.973 | 0.954 | 0.896 | 1 |
| RETN | | | | | | | | | |
| IFI27, IFIT1, ISG15, SLC1A2, IL1R2, FAM20A, | 0.879 | 0.870 | 0.889 | 0.927 | 0.878 | 0.977 | 0.926 | 0.842 | 1 |
| RETN | | | | | | | | | |
| IFI27, SIGLEC1, ISG15, SLC1A2, FAM20A, | 0.879 | 0.869 | 0.890 | 0.922 | 0.864 | 0.980 | 0.952 | 0.878 | 1 |
| RETN, MMP8 | | | | | | | | | |
| IFIT1, SIGLEC1, HERC6, SLC1A2, FAM20A, | 0.879 | 0.870 | 0.889 | 0.920 | 0.864 | 0.975 | 0.948 | 0.877 | 1 |
| RETN, MMP8 | | | | | | | | | |
| RSAD2, IFI27, IFI44L, SLC1A2, IL1R2, OLAH, | 0.879 | 0.871 | 0.888 | 0.923 | 0.871 | 0.976 | 0.934 | 0.866 | 1 |
| RETN | | | | | | | | | |
| RSAD2, IFI27, IFIT1, SLC1A2, IL1R2, FAM20A, | 0.879 | 0.870 | 0.888 | 0.926 | 0.876 | 0.977 | 0.929 | 0.847 | 1 |
| RETN | | | | | | | | | |
| SIGLEC1, SLC1A2, IL1R2, OLAH, FAM20A, | 0.879 | 0.870 | 0.889 | 0.923 | 0.872 | 0.973 | 0.958 | 0.892 | 1 |
| RETN, MMP8 | | | | | | | | | |
| RSAD2, IFI27, OAS1, IFIT1, SLC1A2, IL1R2, | 0.879 | 0.870 | 0.888 | 0.925 | 0.876 | 0.975 | 0.924 | 0.838 | 1 |
| FAM20A | | | | | | | | | |
| RSAD2, IFIT1, SIGLEC1, ISG15, IL1R2, OLAH, | 0.879 | 0.870 | 0.888 | 0.910 | 0.846 | 0.974 | 0.961 | 0.911 | 1 |
| FAM20A | | | | | | | | | |
| RSAD2, IFI27, IFIT1, ISG15, SLC1A2, OLAH, | 0.879 | 0.871 | 0.888 | 0.927 | 0.876 | 0.979 | 0.934 | 0.866 | 1 |
| RETN | | | | | | | | | |
| IFI27, OAS1, IFIT1, SIGLEC1, ISG15, SLC1A2, | 0.879 | 0.870 | 0.889 | 0.922 | 0.867 | 0.977 | 0.941 | 0.867 | 1 |
| FAM20A | | | | | | | | | |
| RSAD2, IFI27, ISG15, SLC1A2, IL1R2, FAM20A, | 0.879 | 0.870 | 0.888 | 0.929 | 0.88 | 0.977 | 0.923 | 0.836 | 1 |
| RETN | | | | | | | | | |
| RSAD2, IFI27, IFI44L, ISG15, SLC1A2, OLAH, | 0.879 | 0.871 | 0.888 | 0.928 | 0.877 | 0.979 | 0.933 | 0.866 | 1.000 |
| RETN | | | | | | | | | |
| RSAD2, IFI27, IFI44L, SLC1A2, IL1R2, | 0.879 | 0.870 | 0.889 | 0.927 | 0.877 | 0.977 | 0.925 | 0.840 | 1 |
| FAM20A, RETN | | | | | | | | | |
| RSAD2, IFI27, IFI44L, SIGLEC1, ISG15, IL1R2, | 0.879 | 0.871 | 0.887 | 0.919 | 0.869 | 0.970 | 0.940 | 0.888 | 0.992 |
| MMP8 | | | | | | | | | |
| RSAD2, IFI27, OAS1, IFIT1, IFI44L, SLC1A2, | 0.879 | 0.870 | 0.888 | 0.927 | 0.875 | 0.979 | 0.941 | 0.879 | 1 |
| OLAH | | | | | | | | | |
| RSAD2, IFI27, IFIT1, IFI44L, HERC6, IL1R2, | 0.879 | 0.871 | 0.887 | 0.918 | 0.868 | 0.969 | 0.937 | 0.882 | 0.992 |
| MMP8 | | | | | | | | | |
| RSAD2, IFI27, OAS1, ISG15, SLC1A2, OLAH, | 0.879 | 0.870 | 0.888 | 0.927 | 0.875 | 0.979 | 0.932 | 0.860 | 1 |
| RETN | | | | | | | | | |
| RSAD2, IFI27, IFIT1, IFI44L, SIGLEC1, ISG15, | 0.879 | 0.871 | 0.887 | 0.918 | 0.866 | 0.969 | 0.943 | 0.893 | 0.994 |
| IL1R2 | | | | | | | | | |
| IFI27, OAS1, SIGLEC1, ISG15, HERC6, SLC1A2, | 0.879 | 0.871 | 0.888 | 0.927 | 0.882 | 0.971 | 0.949 | 0.899 | 0.999 |
| IL1R2 | | | | | | | | | |
| RSAD2, IFI27, IFIT1, IFI44L, ISG15, OLAH, | 0.879 | 0.870 | 0.888 | 0.916 | 0.858 | 0.974 | 0.936 | 0.876 | 0.996 |
| MMP8 | | | | | | | | | |
| IFI27, OAS1, IFIT1, IFI44L, ISG15, SLC1A2, | 0.879 | 0.870 | 0.888 | 0.927 | 0.875 | 0.978 | 0.929 | 0.862 | 0.997 |
| OLAH | | | | | | | | | |
| IFI27, IFI44L, SIGLEC1, ISG15, HERC6, RETN, | 0.879 | 0.870 | 0.889 | 0.911 | 0.849 | 0.973 | 0.941 | 0.884 | 0.999 |
| MMP8 | | | | | | | | | |
| OAS1, SIGLEC1, ISG15, SLC1A2, IL1R2, | 0.879 | 0.870 | 0.889 | 0.924 | 0.876 | 0.972 | 0.959 | 0.893 | 1 |
| FAM20A, MMP8 | | | | | | | | | |
| RSAD2, IFI27, IFIT1, ISG15, SLC1A2, IL1R2, | 0.879 | 0.870 | 0.888 | 0.924 | 0.874 | 0.974 | 0.926 | 0.841 | 1 |
| FAM20A | | | | | | | | | |
| IFI27, OAS1, ISG15, HERC6, SLC1A2, IL1R2, | 0.879 | 0.870 | 0.888 | 0.926 | 0.877 | 0.975 | 0.933 | 0.863 | 1 |
| RETN | | | | | | | | | |
| RSAD2, OAS1, IFIT1, IFI44L, SIGLEC1, IL1R2, | 0.879 | 0.870 | 0.888 | 0.912 | 0.850 | 0.975 | 0.963 | 0.914 | 1 |
| FAM20A | | | | | | | | | |
| IFI27, IFIT1, IFI44L, ISG15, IL1R2, RETN, | 0.879 | 0.870 | 0.888 | 0.919 | 0.863 | 0.974 | 0.943 | 0.887 | 1.000 |
| MMP8 | | | | | | | | | |
| IFI27, OAS1, IFI44L, SIGLEC1, SLC1A2, IL1R2, | 0.879 | 0.871 | 0.888 | 0.924 | 0.875 | 0.973 | 0.949 | 0.894 | 1 |
| RETN | | | | | | | | | |
| IFI44L, SIGLEC1, ISG15, SLC1A2, OLAH, | 0.879 | 0.870 | 0.888 | 0.916 | 0.860 | 0.971 | 0.949 | 0.885 | 1 |
| FAM20A, MMP8 | | | | | | | | | |
| OAS1, IFIT1, SIGLEC1, OLAH, FAM20A, RETN, | 0.879 | 0.869 | 0.889 | 0.912 | 0.848 | 0.977 | 0.960 | 0.911 | 1 |
| MMP8 | | | | | | | | | |
| IFI27, OAS1, IFIT1, ISG15, HERC6, IL1R2, | 0.879 | 0.871 | 0.887 | 0.917 | 0.868 | 0.966 | 0.939 | 0.885 | 0.993 |
| MMP8 | | | | | | | | | |
| IFI27, OAS1, IFIT1, SIGLEC1, ISG15, IL1R2, | 0.879 | 0.871 | 0.887 | 0.920 | 0.872 | 0.968 | 0.952 | 0.906 | 0.998 |
| MMP8 | | | | | | | | | |
| IFI27, OAS1, SIGLEC1, ISG15, SLC1A2, IL1R2, | 0.879 | 0.871 | 0.888 | 0.925 | 0.877 | 0.974 | 0.951 | 0.895 | 1 |
| RETN | | | | | | | | | |
| RSAD2, IFI27, OAS1, IFI44L, SIGLEC1, ISG15, | 0.879 | 0.871 | 0.887 | 0.920 | 0.870 | 0.971 | 0.948 | 0.900 | 0.996 |
| IL1R2 | | | | | | | | | |
| RSAD2, OAS1, SIGLEC1, ISG15, HERC6, IL1R2, | 0.879 | 0.870 | 0.888 | 0.915 | 0.857 | 0.973 | 0.957 | 0.901 | 1 |
| FAM20A | | | | | | | | | |
| RSAD2, IFI27, OAS1, IFI44L, ISG15, OLAH, | 0.879 | 0.870 | 0.888 | 0.916 | 0.856 | 0.976 | 0.938 | 0.874 | 1 |
| RETN | | | | | | | | | |
| RSAD2, IFI27, OAS1, IFIT1, IFI44L, ISG15, | 0.879 | 0.870 | 0.888 | 0.915 | 0.856 | 0.974 | 0.935 | 0.875 | 0.994 |
| OLAH | | | | | | | | | |
| RSAD2, OAS1, IFIT1, SIGLEC1, ISG15, IL1R2, | 0.879 | 0.870 | 0.888 | 0.912 | 0.848 | 0.975 | 0.964 | 0.916 | 1 |
| FAM20A | | | | | | | | | |
| RSAD2, OAS1, SIGLEC1, SLC1A2, OLAH, | 0.879 | 0.869 | 0.889 | 0.918 | 0.862 | 0.973 | 0.952 | 0.892 | 1 |
| FAM20A, MMP8 | | | | | | | | | |
| IFI27, IFIT1, IFI44L, HERC6, SLC1A2, FAM20A, | 0.879 | 0.869 | 0.889 | 0.922 | 0.865 | 0.978 | 0.930 | 0.847 | 1 |
| RETN | | | | | | | | | |
| IFI27, OAS1, IFI44L, SLC1A2, IL1R2, OLAH, | 0.879 | 0.870 | 0.888 | 0.928 | 0.879 | 0.977 | 0.932 | 0.864 | 0.999 |
| MMP8 | | | | | | | | | |
| OAS1, IFI44L, SIGLEC1, ISG15, HERC6, IL1R2, | 0.879 | 0.870 | 0.888 | 0.914 | 0.855 | 0.973 | 0.958 | 0.904 | 1 |
| FAM20A | | | | | | | | | |
| IFI27, OAS1, IFIT1, IFI44L, SLC1A2, OLAH, | 0.879 | 0.870 | 0.888 | 0.926 | 0.874 | 0.977 | 0.938 | 0.869 | 1 |
| RETN | | | | | | | | | |
| IFIT1, IFI44L, SIGLEC1, SLC1A2, OLAH, | 0.879 | 0.870 | 0.888 | 0.919 | 0.863 | 0.975 | 0.937 | 0.874 | 1 |
| FAM20A, RETN | | | | | | | | | |
| OAS1, SIGLEC1, SLC1A2, IL1R2, FAM20A, | 0.879 | 0.869 | 0.888 | 0.923 | 0.873 | 0.973 | 0.958 | 0.894 | 1 |
| RETN, MMP8 | | | | | | | | | |
| RSAD2, IFI27, OAS1, IFIT1, ISG15, HERC6, | 0.879 | 0.871 | 0.887 | 0.918 | 0.870 | 0.966 | 0.942 | 0.890 | 0.995 |
| IL1R2 | | | | | | | | | |
| IFI27, OAS1, IFIT1, SIGLEC1, SLC1A2, | 0.879 | 0.869 | 0.888 | 0.921 | 0.864 | 0.978 | 0.942 | 0.870 | 1 |
| FAM20A, MMP8 | | | | | | | | | |
| RSAD2, IFI27, IFIT1, IFI44L, ISG15, OLAH, | 0.879 | 0.870 | 0.887 | 0.915 | 0.856 | 0.975 | 0.939 | 0.875 | 1 |
| RETN | | | | | | | | | |
| RSAD2, IFI27, OAS1, IFI44L, SIGLEC1, | 0.879 | 0.869 | 0.889 | 0.913 | 0.849 | 0.978 | 0.948 | 0.878 | 1 |
| FAM20A, MMP8 | | | | | | | | | |
| IFIT1, IFI44L, SIGLEC1, OLAH, FAM20A, | 0.879 | 0.869 | 0.888 | 0.915 | 0.853 | 0.978 | 0.957 | 0.907 | 1 |
| RETN, MMP8 | | | | | | | | | |
| IFIT1, IFI44L, SIGLEC1, IL1R2, OLAH, | 0.879 | 0.870 | 0.888 | 0.908 | 0.844 | 0.971 | 0.959 | 0.907 | 1 |
| FAM20A, RETN | | | | | | | | | |
| IFI27, SIGLEC1, ISG15, SLC1A2, IL1R2, RETN, | 0.879 | 0.870 | 0.887 | 0.926 | 0.878 | 0.973 | 0.965 | 0.925 | 1 |
| MMP8 | | | | | | | | | |
| RSAD2, IFI27, OAS1, IFIT1, IFI44L, IL1R2, | 0.879 | 0.870 | 0.887 | 0.915 | 0.860 | 0.970 | 0.945 | 0.887 | 1 |
| RETN | | | | | | | | | |
| RSAD2, IFI27, OAS1, SIGLEC1, ISG15, | 0.879 | 0.868 | 0.889 | 0.915 | 0.851 | 0.979 | 0.947 | 0.876 | 1 |
| FAM20A, MMP8 | | | | | | | | | |
| RSAD2, IFI27, IFI44L, SIGLEC1, SLC1A2, | 0.879 | 0.869 | 0.888 | 0.922 | 0.865 | 0.979 | 0.945 | 0.871 | 1 |
| FAM20A, RETN | | | | | | | | | |
| RSAD2, IFI27, OAS1, IFIT1, SIGLEC1, IL1R2, | 0.879 | 0.870 | 0.887 | 0.920 | 0.872 | 0.968 | 0.957 | 0.912 | 1 |
| MMP8 | | | | | | | | | |
| RSAD2, IFIT1, IFI44L, SIGLEC1, HERC6, | 0.879 | 0.870 | 0.888 | 0.915 | 0.855 | 0.976 | 0.934 | 0.866 | 1 |
| FAM20A, RETN | | | | | | | | | |
| OAS1, IFIT1, IFI44L, SIGLEC1, SLC1A2, OLAH, | 0.879 | 0.869 | 0.888 | 0.920 | 0.865 | 0.975 | 0.934 | 0.868 | 0.999 |
| FAM20A | | | | | | | | | |
| RSAD2, IFI27, IFIT1, SIGLEC1, ISG15, IL1R2, | 0.879 | 0.870 | 0.887 | 0.920 | 0.866 | 0.973 | 0.955 | 0.910 | 1 |
| RETN | | | | | | | | | |
| RSAD2, OAS1, SIGLEC1, HERC6, IL1R2, | 0.879 | 0.869 | 0.888 | 0.914 | 0.856 | 0.973 | 0.958 | 0.903 | 1 |
| OLAH, FAM20A | | | | | | | | | |
| RSAD2, IFI44L, SIGLEC1, IL1R2, FAM20A, | 0.879 | 0.869 | 0.888 | 0.919 | 0.862 | 0.976 | 0.964 | 0.911 | 1 |
| RETN, MMP8 | | | | | | | | | |
| IFI27, OAS1, ISG15, SLC1A2, IL1R2, FAM20A, | 0.879 | 0.870 | 0.888 | 0.929 | 0.880 | 0.978 | 0.923 | 0.836 | 1 |
| RETN | | | | | | | | | |
| OAS1, IFIT1, IFI44L, SIGLEC1, IL1R2, | 0.879 | 0.869 | 0.888 | 0.910 | 0.847 | 0.973 | 0.961 | 0.908 | 1 |
| FAM20A, RETN | | | | | | | | | |
| RSAD2, IFIT1, IFI44L, SIGLEC1, OLAH, | 0.879 | 0.869 | 0.888 | 0.908 | 0.842 | 0.974 | 0.955 | 0.906 | 1 |
| FAM20A, MMP8 | | | | | | | | | |
| RSAD2, IFI27, IFIT1, IFI44L, ISG15, SLC1A2, | 0.879 | 0.870 | 0.887 | 0.926 | 0.873 | 0.978 | 0.928 | 0.861 | 0.996 |
| OLAH | | | | | | | | | |
| IFIT1, IFI44L, SIGLEC1, SLC1A2, IL1R2, OLAH, | 0.879 | 0.869 | 0.888 | 0.920 | 0.867 | 0.974 | 0.946 | 0.886 | 1 |
| FAM20A | | | | | | | | | |
| IFI27, SIGLEC1, HERC6, SLC1A2, FAM20A, | 0.878 | 0.868 | 0.889 | 0.924 | 0.866 | 0.982 | 0.951 | 0.875 | 1 |
| RETN, MMP8 | | | | | | | | | |
| RSAD2, IFI27, OAS1, ISG15, SLC1A2, IL1R2, | 0.878 | 0.869 | 0.888 | 0.927 | 0.879 | 0.974 | 0.926 | 0.840 | 1 |
| FAM20A | | | | | | | | | |
| RSAD2, SIGLEC1, HERC6, SLC1A2, OLAH, | 0.878 | 0.868 | 0.888 | 0.922 | 0.871 | 0.973 | 0.945 | 0.878 | 1 |
| FAM20A, MMP8 | | | | | | | | | |
| SIGLEC1, HERC6, SLC1A2, IL1R2, FAM20A, | 0.878 | 0.869 | 0.888 | 0.922 | 0.872 | 0.973 | 0.957 | 0.889 | 1 |
| RETN, MMP8 | | | | | | | | | |
| IFI27, SIGLEC1, ISG15, HERC6, SLC1A2, | 0.878 | 0.868 | 0.889 | 0.920 | 0.862 | 0.978 | 0.943 | 0.866 | 1 |
| FAM20A, RETN | | | | | | | | | |
| IFI27, OAS1, IFI44L, HERC6, SLC1A2, IL1R2, | 0.878 | 0.870 | 0.887 | 0.924 | 0.876 | 0.973 | 0.935 | 0.869 | 1 |
| RETN | | | | | | | | | |
| RSAD2, IFI27, OAS1, HERC6, SLC1A2, | 0.878 | 0.868 | 0.888 | 0.923 | 0.868 | 0.979 | 0.933 | 0.849 | 1 |
| FAM20A, RETN | | | | | | | | | |
| RSAD2, IFI27, OAS1, ISG15, HERC6, IL1R2, | 0.878 | 0.870 | 0.887 | 0.921 | 0.873 | 0.968 | 0.934 | 0.876 | 0.992 |
| MMP8 | | | | | | | | | |
| IFIT1, SIGLEC1, HERC6, SLC1A2, OLAH, | 0.878 | 0.868 | 0.888 | 0.919 | 0.866 | 0.973 | 0.935 | 0.861 | 1 |
| FAM20A, RETN | | | | | | | | | |
| RSAD2, IFI27, OAS1, SLC1A2, IL1R2, FAM20A, | 0.878 | 0.869 | 0.887 | 0.927 | 0.879 | 0.976 | 0.922 | 0.834 | 1 |
| RETN | | | | | | | | | |
| IFIT1, SIGLEC1, HERC6, SLC1A2, IL1R2, | 0.878 | 0.869 | 0.888 | 0.924 | 0.875 | 0.973 | 0.946 | 0.874 | 1 |
| FAM20A, RETN | | | | | | | | | |
| RSAD2, IFI27, IFIT1, SIGLEC1, ISG15, SLC1A2, | 0.878 | 0.869 | 0.887 | 0.922 | 0.867 | 0.978 | 0.940 | 0.864 | 1 |
| FAM20A | | | | | | | | | |
| IFI27, OAS1, IFI44L, ISG15, HERC6, IL1R2, | 0.878 | 0.870 | 0.886 | 0.918 | 0.870 | 0.967 | 0.928 | 0.868 | 0.988 |
| MMP8 | | | | | | | | | |
| IFI27, IFIT1, IFI44L, HERC6, SLC1A2, IL1R2, | 0.878 | 0.869 | 0.888 | 0.925 | 0.875 | 0.975 | 0.933 | 0.870 | 0.995 |
| OLAH | | | | | | | | | |
| IFI27, OAS1, IFI44L, ISG15, SLC1A2, OLAH, | 0.878 | 0.869 | 0.887 | 0.929 | 0.878 | 0.979 | 0.933 | 0.865 | 1 |
| RETN | | | | | | | | | |
| IFI27, OAS1, SIGLEC1, HERC6, SLC1A2, | 0.878 | 0.868 | 0.888 | 0.920 | 0.866 | 0.974 | 0.941 | 0.865 | 1 |
| FAM20A, MMP8 | | | | | | | | | |
| RSAD2, IFI27, OAS1, IFI44L, SLC1A2, IL1R2, | 0.878 | 0.869 | 0.887 | 0.927 | 0.877 | 0.977 | 0.932 | 0.863 | 1.000 |
| OLAH | | | | | | | | | |
| RSAD2, IFIT1, IFI44L, SIGLEC1, SLC1A2, | 0.878 | 0.869 | 0.887 | 0.921 | 0.866 | 0.976 | 0.938 | 0.875 | 1 |
| OLAH, FAM20A | | | | | | | | | |
| RSAD2, IFI27, OAS1, IFI44L, SLC1A2, OLAH, | 0.878 | 0.869 | 0.887 | 0.926 | 0.873 | 0.979 | 0.938 | 0.870 | 1 |
| RETN | | | | | | | | | |
| IFI27, IFIT1, ISG15, HERC6, SLC1A2, FAM20A, | 0.878 | 0.868 | 0.888 | 0.920 | 0.864 | 0.977 | 0.936 | 0.855 | 1 |
| RETN | | | | | | | | | |
| RSAD2, OAS1, IFI44L, SIGLEC1, HERC6, | 0.878 | 0.869 | 0.887 | 0.916 | 0.857 | 0.974 | 0.959 | 0.905 | 1 |
| IL1R2, FAM20A | | | | | | | | | |
| RSAD2, OAS1, IFIT1, SIGLEC1, ISG15, HERC6, | 0.878 | 0.869 | 0.887 | 0.910 | 0.849 | 0.971 | 0.938 | 0.868 | 1 |
| FAM20A | | | | | | | | | |
| IFI27, OAS1, IFIT1, IFI44L, SLC1A2, IL1R2, | 0.878 | 0.869 | 0.887 | 0.926 | 0.875 | 0.976 | 0.932 | 0.864 | 0.999 |
| OLAH | | | | | | | | | |
| IFI27, OAS1, IFIT1, SIGLEC1, SLC1A2, IL1R2, | 0.878 | 0.870 | 0.886 | 0.926 | 0.882 | 0.971 | 0.953 | 0.905 | 1 |
| MMP8 | | | | | | | | | |
| IFI27, OAS1, IFIT1, HERC6, SLC1A2, FAM20A, | 0.878 | 0.868 | 0.888 | 0.923 | 0.866 | 0.979 | 0.934 | 0.851 | 1 |
| RETN | | | | | | | | | |
| RSAD2, IFI27, IFIT1, IFI44L, IL1R2, RETN, | 0.878 | 0.869 | 0.887 | 0.918 | 0.862 | 0.973 | 0.943 | 0.887 | 1.000 |
| MMP8 | | | | | | | | | |
| RSAD2, IFI27, IFI44L, SIGLEC1, ISG15, | 0.878 | 0.868 | 0.888 | 0.913 | 0.849 | 0.977 | 0.943 | 0.870 | 1 |
| FAM20A, MMP8 | | | | | | | | | |
| RSAD2, IFIT1, IFI44L, SIGLEC1, ISG15, IL1R2, | 0.878 | 0.869 | 0.887 | 0.911 | 0.847 | 0.974 | 0.963 | 0.914 | 1 |
| FAM20A | | | | | | | | | |
| IFI27, OAS1, IFIT1, IFI44L, ISG15, IL1R2, RETN | 0.878 | 0.869 | 0.887 | 0.919 | 0.865 | 0.972 | 0.941 | 0.883 | 0.999 |
| IFIT1, IFI44L, SIGLEC1, ISG15, IL1R2, | 0.878 | 0.869 | 0.887 | 0.910 | 0.847 | 0.972 | 0.960 | 0.909 | 1 |
| FAM20A, RETN | | | | | | | | | |
| RSAD2, IFI27, IFIT1, IFI44L, ISG15, HERC6, | 0.878 | 0.869 | 0.887 | 0.914 | 0.855 | 0.973 | 0.940 | 0.883 | 0.998 |
| RETN | | | | | | | | | |
| IFI27, OAS1, IFI44L, SLC1A2, IL1R2, OLAH, | 0.878 | 0.869 | 0.887 | 0.926 | 0.875 | 0.978 | 0.935 | 0.867 | 1 |
| RETN | | | | | | | | | |
| RSAD2, OAS1, IFIT1, IFI44L, SIGLEC1, HERC6, | 0.878 | 0.869 | 0.887 | 0.910 | 0.848 | 0.971 | 0.932 | 0.860 | 1 |
| FAM20A | | | | | | | | | |
| OAS1, IFIT1, SIGLEC1, ISG15, IL1R2, FAM20A, | 0.878 | 0.869 | 0.887 | 0.912 | 0.850 | 0.974 | 0.961 | 0.908 | 1 |
| RETN | | | | | | | | | |
| RSAD2, IFI27, IFI44L, SIGLEC1, SLC1A2, | 0.878 | 0.870 | 0.886 | 0.923 | 0.873 | 0.974 | 0.949 | 0.896 | 1 |
| IL1R2, RETN | | | | | | | | | |
| RSAD2, IFI27, ISG15, HERC6, SLC1A2, | 0.878 | 0.868 | 0.888 | 0.922 | 0.867 | 0.978 | 0.934 | 0.852 | 1 |
| FAM20A, RETN | | | | | | | | | |
| IFI27, IFIT1, IFI44L, SIGLEC1, ISG15, SLC1A2, | 0.878 | 0.870 | 0.886 | 0.925 | 0.879 | 0.970 | 0.946 | 0.895 | 0.997 |
| IL1R2 | | | | | | | | | |
| RSAD2, IFI44L, SIGLEC1, ISG15, HERC6, | 0.878 | 0.868 | 0.887 | 0.915 | 0.856 | 0.974 | 0.958 | 0.901 | 1 |
| IL1R2, FAM20A | | | | | | | | | |
| RSAD2, IFI27, SIGLEC1, HERC6, SLC1A2, | 0.878 | 0.869 | 0.887 | 0.924 | 0.879 | 0.970 | 0.949 | 0.900 | 0.998 |
| IL1R2, MMP8 | | | | | | | | | |
| OAS1, IFIT1, SIGLEC1, SLC1A2, IL1R2, OLAH, | 0.878 | 0.868 | 0.887 | 0.918 | 0.863 | 0.972 | 0.952 | 0.891 | 1 |
| FAM20A | | | | | | | | | |
| RSAD2, IFIT1, IFI44L, SIGLEC1, ISG15, HERC6, | 0.878 | 0.869 | 0.887 | 0.911 | 0.850 | 0.972 | 0.935 | 0.866 | 1 |
| FAM20A | | | | | | | | | |
| RSAD2, IFI27, IFIT1, IFI44L, SLC1A2, OLAH, | 0.878 | 0.869 | 0.887 | 0.923 | 0.871 | 0.976 | 0.939 | 0.875 | 1 |
| RETN | | | | | | | | | |
| RSAD2, IFI27, IFIT1, IFI44L, SLC1A2, IL1R2, | 0.878 | 0.869 | 0.887 | 0.925 | 0.874 | 0.976 | 0.933 | 0.868 | 0.997 |
| OLAH | | | | | | | | | |
| IFI27, OAS1, IFIT1, IFI44L, ISG15, HERC6, | 0.878 | 0.869 | 0.886 | 0.921 | 0.873 | 0.970 | 0.936 | 0.879 | 0.992 |
| IL1R2 | | | | | | | | | |
| RSAD2, IFI27, OAS1, SIGLEC1, HERC6, | 0.878 | 0.868 | 0.888 | 0.920 | 0.866 | 0.975 | 0.940 | 0.864 | 1 |
| SLC1A2, FAM20A | | | | | | | | | |
| RSAD2, SIGLEC1, ISG15, HERC6, IL1R2, | 0.878 | 0.868 | 0.887 | 0.916 | 0.858 | 0.974 | 0.955 | 0.898 | 1 |
| OLAH, FAM20A | | | | | | | | | |
| IFI27, OAS1, IFI44L, SIGLEC1, ISG15, FAM20A, | 0.878 | 0.867 | 0.888 | 0.911 | 0.847 | 0.975 | 0.946 | 0.874 | 1 |
| MMP8 | | | | | | | | | |
| OAS1, SIGLEC1, HERC6, SLC1A2, OLAH, | 0.878 | 0.868 | 0.888 | 0.919 | 0.866 | 0.972 | 0.943 | 0.876 | 1 |
| FAM20A, MMP8 | | | | | | | | | |
| RSAD2, IFI44L, SIGLEC1, HERC6, IL1R2, | 0.878 | 0.868 | 0.887 | 0.914 | 0.855 | 0.974 | 0.958 | 0.901 | 1 |
| OLAH, FAM20A | | | | | | | | | |
| IFIT1, SIGLEC1, SLC1A2, OLAH, FAM20A, | 0.878 | 0.868 | 0.887 | 0.922 | 0.868 | 0.975 | 0.952 | 0.894 | 1 |
| RETN, MMP8 | | | | | | | | | |
| IFI27, OAS1, IFIT1, ISG15, HERC6, SLC1A2, | 0.878 | 0.868 | 0.887 | 0.915 | 0.857 | 0.973 | 0.933 | 0.858 | 1 |
| RETN | | | | | | | | | |
| RSAD2, IFI27, IFIT1, SIGLEC1, HERC6, | 0.878 | 0.869 | 0.886 | 0.921 | 0.869 | 0.974 | 0.940 | 0.883 | 0.998 |
| SLC1A2, RETN | | | | | | | | | |
| IFIT1, IFI44L, SIGLEC1, HERC6, SLC1A2, | 0.878 | 0.869 | 0.887 | 0.923 | 0.871 | 0.974 | 0.933 | 0.857 | 1 |
| FAM20A, RETN | | | | | | | | | |
| OAS1, IFIT1, IFI44L, SIGLEC1, ISG15, OLAH, | 0.878 | 0.868 | 0.887 | 0.905 | 0.836 | 0.974 | 0.947 | 0.891 | 1 |
| FAM20A | | | | | | | | | |
| RSAD2, IFI27, OAS1, IFIT1, SIGLEC1, SLC1A2, | 0.878 | 0.868 | 0.887 | 0.920 | 0.863 | 0.977 | 0.946 | 0.874 | 1 |
| FAM20A | | | | | | | | | |
| RSAD2, IFI27, OAS1, ISG15, HERC6, SLC1A2, | 0.878 | 0.868 | 0.887 | 0.915 | 0.856 | 0.975 | 0.928 | 0.850 | 1 |
| RETN | | | | | | | | | |
| RSAD2, IFI27, SIGLEC1, HERC6, SLC1A2, | 0.878 | 0.868 | 0.888 | 0.921 | 0.866 | 0.976 | 0.940 | 0.864 | 1 |
| FAM20A, MMP8 | | | | | | | | | |
| RSAD2, IFI27, IFIT1, SIGLEC1, SLC1A2, IL1R2, | 0.878 | 0.869 | 0.886 | 0.925 | 0.877 | 0.973 | 0.953 | 0.903 | 1 |
| RETN | | | | | | | | | |
| IFI27, OAS1, IFIT1, SIGLEC1, ISG15, SLC1A2, | 0.878 | 0.869 | 0.886 | 0.925 | 0.880 | 0.970 | 0.950 | 0.901 | 0.999 |
| IL1R2 | | | | | | | | | |
| IFIT1, IFI44L, SIGLEC1, ISG15, HERC6, | 0.878 | 0.868 | 0.887 | 0.920 | 0.867 | 0.973 | 0.934 | 0.856 | 1 |
| SLC1A2, FAM20A | | | | | | | | | |
| RSAD2, SIGLEC1, ISG15, SLC1A2, OLAH, | 0.877 | 0.868 | 0.887 | 0.917 | 0.863 | 0.971 | 0.951 | 0.891 | 1 |
| FAM20A, MMP8 | | | | | | | | | |
| IFI27, SIGLEC1, HERC6, SLC1A2, IL1R2, | 0.877 | 0.868 | 0.887 | 0.925 | 0.877 | 0.973 | 0.965 | 0.926 | 1 |
| RETN, MMP8 | | | | | | | | | |
| RSAD2, IFI27, IFI44L, ISG15, HERC6, IL1R2, | 0.877 | 0.870 | 0.885 | 0.916 | 0.866 | 0.966 | 0.936 | 0.880 | 0.992 |
| MMP8 | | | | | | | | | |
| RSAD2, OAS1, IFIT1, SIGLEC1, HERC6, | 0.877 | 0.868 | 0.887 | 0.911 | 0.849 | 0.972 | 0.937 | 0.865 | 1 |
| FAM20A, MMP8 | | | | | | | | | |
| IFI27, OAS1, IFIT1, ISG15, IL1R2, RETN, MMP8 | 0.877 | 0.868 | 0.887 | 0.920 | 0.865 | 0.974 | 0.951 | 0.899 | 1 |
| IFI27, OAS1, IFI44L, SIGLEC1, HERC6, | 0.877 | 0.868 | 0.887 | 0.920 | 0.865 | 0.974 | 0.942 | 0.866 | 1 |
| SLC1A2, FAM20A | | | | | | | | | |
| RSAD2, IFI27, OAS1, IFI44L, ISG15, HERC6, | 0.877 | 0.869 | 0.886 | 0.922 | 0.874 | 0.969 | 0.930 | 0.871 | 0.990 |
| IL1R2 | | | | | | | | | |
| IFI27, OAS1, IFI44L, ISG15, SLC1A2, IL1R2, | 0.877 | 0.868 | 0.887 | 0.928 | 0.878 | 0.977 | 0.923 | 0.851 | 0.994 |
| OLAH | | | | | | | | | |
| RSAD2, IFI27, IFIT1, SIGLEC1, SLC1A2, | 0.877 | 0.868 | 0.887 | 0.921 | 0.864 | 0.977 | 0.943 | 0.870 | 1 |
| FAM20A, MMP8 | | | | | | | | | |
| IFIT1, IFI44L, SIGLEC1, HERC6, SLC1A2, | 0.877 | 0.868 | 0.887 | 0.919 | 0.866 | 0.972 | 0.934 | 0.856 | 1 |
| FAM20A, MMP8 | | | | | | | | | |
| RSAD2, IFI27, SIGLEC1, ISG15, SLC1A2, | 0.877 | 0.869 | 0.886 | 0.924 | 0.875 | 0.973 | 0.953 | 0.904 | 1 |
| IL1R2, RETN | | | | | | | | | |
| RSAD2, IFIT1, SIGLEC1, ISG15, OLAH, | 0.877 | 0.868 | 0.887 | 0.908 | 0.841 | 0.974 | 0.954 | 0.902 | 1 |
| FAM20A, MMP8 | | | | | | | | | |
| IFI27, OAS1, IFI44L, SIGLEC1, HERC6, | 0.877 | 0.868 | 0.886 | 0.926 | 0.882 | 0.970 | 0.948 | 0.899 | 0.997 |
| SLC1A2, IL1R2 | | | | | | | | | |
| RSAD2, IFIT1, SIGLEC1, SLC1A2, IL1R2, | 0.877 | 0.868 | 0.886 | 0.919 | 0.864 | 0.973 | 0.954 | 0.892 | 1 |
| OLAH, FAM20A | | | | | | | | | |
| IFI27, IFIT1, SIGLEC1, HERC6, SLC1A2, IL1R2, | 0.877 | 0.869 | 0.886 | 0.925 | 0.879 | 0.971 | 0.951 | 0.904 | 0.998 |
| MMP8 | | | | | | | | | |
| RSAD2, OAS1, IFIT1, SIGLEC1, IL1R2, | 0.877 | 0.868 | 0.886 | 0.911 | 0.848 | 0.975 | 0.963 | 0.911 | 1 |
| FAM20A, RETN | | | | | | | | | |
| RSAD2, IFI27, OAS1, IFIT1, SIGLEC1, ISG15, | 0.877 | 0.869 | 0.886 | 0.921 | 0.872 | 0.969 | 0.955 | 0.911 | 1.000 |
| IL1R2 | | | | | | | | | |
| IFI27, OAS1, IFI44L, ISG15, HERC6, SLC1A2, | 0.877 | 0.868 | 0.887 | 0.915 | 0.857 | 0.973 | 0.933 | 0.858 | 1 |
| RETN | | | | | | | | | |
| RSAD2, IFIT1, SIGLEC1, IL1R2, OLAH, | 0.877 | 0.868 | 0.886 | 0.911 | 0.848 | 0.974 | 0.964 | 0.916 | 1 |
| FAM20A, RETN | | | | | | | | | |
| OAS1, IFIT1, IFI44L, SIGLEC1, HERC6, | 0.877 | 0.868 | 0.887 | 0.910 | 0.848 | 0.971 | 0.936 | 0.867 | 1 |
| FAM20A, MMP8 | | | | | | | | | |
| IFI27, OAS1, SIGLEC1, ISG15, HERC6, SLC1A2, | 0.877 | 0.867 | 0.887 | 0.920 | 0.866 | 0.975 | 0.945 | 0.869 | 1 |
| FAM20A | | | | | | | | | |
| OAS1, IFIT1, IFI44L, SIGLEC1, ISG15, HERC6, | 0.877 | 0.868 | 0.886 | 0.913 | 0.853 | 0.973 | 0.939 | 0.872 | 1 |
| FAM20A | | | | | | | | | |
| IFIT1, IFI44L, SIGLEC1, ISG15, SLC1A2, IL1R2, | 0.877 | 0.868 | 0.886 | 0.921 | 0.869 | 0.972 | 0.948 | 0.888 | 1 |
| FAM20A | | | | | | | | | |
| SIGLEC1, ISG15, HERC6, SLC1A2, OLAH, | 0.877 | 0.867 | 0.887 | 0.919 | 0.865 | 0.973 | 0.947 | 0.876 | 1 |
| FAM20A, MMP8 | | | | | | | | | |
| RSAD2, IFI27, OAS1, SIGLEC1, ISG15, IL1R2, | 0.877 | 0.868 | 0.886 | 0.921 | 0.873 | 0.969 | 0.957 | 0.913 | 1 |
| MMP8 | | | | | | | | | |
| OAS1, IFI44L, SIGLEC1, HERC6, IL1R2, OLAH, | 0.877 | 0.868 | 0.887 | 0.914 | 0.856 | 0.973 | 0.959 | 0.905 | 1 |
| FAM20A | | | | | | | | | |
| IFIT1, SIGLEC1, ISG15, IL1R2, OLAH, | 0.877 | 0.868 | 0.886 | 0.910 | 0.848 | 0.972 | 0.960 | 0.908 | 1 |
| FAM20A, RETN | | | | | | | | | |
| IFI27, IFIT1, SIGLEC1, ISG15, SLC1A2, IL1R2, | 0.877 | 0.869 | 0.885 | 0.924 | 0.878 | 0.969 | 0.950 | 0.901 | 0.999 |
| MMP8 | | | | | | | | | |
| RSAD2, IFI27, OAS1, IFIT1, SIGLEC1, SLC1A2, | 0.877 | 0.869 | 0.885 | 0.924 | 0.878 | 0.971 | 0.954 | 0.905 | 1 |
| IL1R2 | | | | | | | | | |
| RSAD2, IFI27, IFI44L, SIGLEC1, HERC6, | 0.877 | 0.867 | 0.887 | 0.921 | 0.867 | 0.976 | 0.940 | 0.864 | 1 |
| SLC1A2, FAM20A | | | | | | | | | |
| RSAD2, IFI27, IFIT1, SIGLEC1, HERC6, | 0.877 | 0.869 | 0.885 | 0.926 | 0.880 | 0.971 | 0.950 | 0.902 | 0.998 |
| SLC1A2, IL1R2 | | | | | | | | | |
| IFI27, IFIT1, IFI44L, SIGLEC1, SLC1A2, IL1R2, | 0.877 | 0.869 | 0.885 | 0.926 | 0.880 | 0.971 | 0.951 | 0.905 | 0.997 |
| MMP8 | | | | | | | | | |
| RSAD2, IFI27, IFIT1, IFI44L, ISG15, HERC6, | 0.877 | 0.869 | 0.885 | 0.919 | 0.868 | 0.969 | 0.935 | 0.878 | 0.992 |
| IL1R2 | | | | | | | | | |
| IFIT1, SIGLEC1, ISG15, HERC6, SLC1A2, | 0.877 | 0.867 | 0.887 | 0.919 | 0.866 | 0.973 | 0.939 | 0.863 | 1 |
| FAM20A, RETN | | | | | | | | | |
| OAS1, SIGLEC1, ISG15, SLC1A2, OLAH, | 0.877 | 0.867 | 0.887 | 0.917 | 0.862 | 0.971 | 0.951 | 0.892 | 1 |
| FAM20A, MMP8 | | | | | | | | | |
| RSAD2, IFI27, OAS1, IFI44L, HERC6, SLC1A2, | 0.877 | 0.868 | 0.886 | 0.915 | 0.855 | 0.975 | 0.929 | 0.852 | 1 |
| RETN | | | | | | | | | |
| IFI27, IFIT1, IFI44L, ISG15, HERC6, IL1R2, | 0.877 | 0.869 | 0.885 | 0.919 | 0.869 | 0.969 | 0.936 | 0.879 | 0.993 |
| MMP8 | | | | | | | | | |
| RSAD2, OAS1, IFIT1, SIGLEC1, SLC1A2, | 0.877 | 0.867 | 0.886 | 0.920 | 0.865 | 0.975 | 0.950 | 0.890 | 1 |
| OLAH, FAM20A | | | | | | | | | |
| RSAD2, IFI44L, SIGLEC1, SLC1A2, OLAH, | 0.877 | 0.867 | 0.887 | 0.919 | 0.864 | 0.974 | 0.946 | 0.882 | 1 |
| FAM20A, MMP8 | | | | | | | | | |
| RSAD2, IFI27, SIGLEC1, ISG15, HERC6, | 0.877 | 0.867 | 0.887 | 0.921 | 0.867 | 0.976 | 0.940 | 0.864 | 1 |
| SLC1A2, FAM20A | | | | | | | | | |
| IFI27, SIGLEC1, ISG15, HERC6, SLC1A2, | 0.877 | 0.868 | 0.885 | 0.925 | 0.877 | 0.974 | 0.954 | 0.907 | 1 |
| IL1R2, RETN | | | | | | | | | |
| OAS1, IFIT1, IFI44L, SIGLEC1, HERC6, | 0.877 | 0.868 | 0.886 | 0.920 | 0.868 | 0.972 | 0.932 | 0.855 | 1 |
| SLC1A2, FAM20A | | | | | | | | | |
| RSAD2, IFI27, OAS1, IFIT1, ISG15, IL1R2, | 0.877 | 0.868 | 0.886 | 0.916 | 0.862 | 0.971 | 0.950 | 0.897 | 1 |
| RETN | | | | | | | | | |
| OAS1, IFI44L, SIGLEC1, SLC1A2, OLAH, | 0.877 | 0.867 | 0.887 | 0.917 | 0.861 | 0.973 | 0.948 | 0.886 | 1 |
| FAM20A, MMP8 | | | | | | | | | |
| OAS1, SIGLEC1, ISG15, HERC6, IL1R2, OLAH, | 0.877 | 0.867 | 0.886 | 0.913 | 0.854 | 0.972 | 0.959 | 0.905 | 1 |
| FAM20A | | | | | | | | | |
| RSAD2, IFI27, IFIT1, ISG15, HERC6, IL1R2, | 0.877 | 0.868 | 0.885 | 0.916 | 0.866 | 0.966 | 0.939 | 0.885 | 0.994 |
| MMP8 | | | | | | | | | |
| RSAD2, IFI27, OAS1, SIGLEC1, ISG15, SLC1A2, | 0.877 | 0.868 | 0.885 | 0.925 | 0.880 | 0.971 | 0.954 | 0.905 | 1 |
| IL1R2 | | | | | | | | | |
| IFIT1, IFI44L, SIGLEC1, SLC1A2, IL1R2, | 0.877 | 0.868 | 0.886 | 0.919 | 0.866 | 0.971 | 0.950 | 0.891 | 1 |
| FAM20A, RETN | | | | | | | | | |
| IFIT1, SIGLEC1, SLC1A2, IL1R2, OLAH, | 0.877 | 0.867 | 0.886 | 0.920 | 0.867 | 0.972 | 0.952 | 0.895 | 1 |
| FAM20A, RETN | | | | | | | | | |
| OAS1, IFIT1, SIGLEC1, ISG15, HERC6, | 0.877 | 0.867 | 0.886 | 0.910 | 0.849 | 0.971 | 0.941 | 0.873 | 1 |
| FAM20A, MMP8 | | | | | | | | | |
| RSAD2, OAS1, IFIT1, IFI44L, SIGLEC1, OLAH, | 0.877 | 0.867 | 0.886 | 0.905 | 0.835 | 0.974 | 0.948 | 0.893 | 1 |
| FAM20A | | | | | | | | | |
| IFI27, IFIT1, IFI44L, SIGLEC1, HERC6, SLC1A2, | 0.877 | 0.868 | 0.885 | 0.918 | 0.863 | 0.973 | 0.946 | 0.892 | 1.000 |
| RETN | | | | | | | | | |
| RSAD2, IFI27, OAS1, IFIT1, IFI44L, IL1R2, | 0.877 | 0.868 | 0.885 | 0.916 | 0.865 | 0.968 | 0.940 | 0.886 | 0.994 |
| MMP8 | | | | | | | | | |
| IFI27, OAS1, HERC6, SLC1A2, FAM20A, RETN, | 0.877 | 0.867 | 0.887 | 0.923 | 0.866 | 0.980 | 0.934 | 0.851 | 1 |
| MMP8 | | | | | | | | | |
| OAS1, IFIT1, IFI44L, SIGLEC1, SLC1A2, IL1R2, | 0.877 | 0.868 | 0.886 | 0.920 | 0.867 | 0.973 | 0.950 | 0.890 | 1 |
| FAM20A | | | | | | | | | |
| RSAD2, IFI27, IFIT1, IFI44L, ISG15, IL1R2, | 0.877 | 0.868 | 0.885 | 0.916 | 0.861 | 0.971 | 0.939 | 0.880 | 0.998 |
| RETN | | | | | | | | | |
| RSAD2, OAS1, IFIT1, SIGLEC1, ISG15, OLAH, | 0.877 | 0.867 | 0.886 | 0.905 | 0.836 | 0.975 | 0.952 | 0.901 | 1 |
| FAM20A | | | | | | | | | |
| OAS1, IFIT1, SIGLEC1, ISG15, SLC1A2, OLAH, | 0.877 | 0.867 | 0.886 | 0.918 | 0.863 | 0.974 | 0.945 | 0.882 | 1 |
| FAM20A | | | | | | | | | |
| RSAD2, IFIT1, SIGLEC1, ISG15, HERC6, | 0.877 | 0.867 | 0.886 | 0.911 | 0.850 | 0.972 | 0.936 | 0.862 | 1 |
| FAM20A, MMP8 | | | | | | | | | |
| IFI27, IFI44L, ISG15, HERC6, SLC1A2, | 0.876 | 0.866 | 0.886 | 0.919 | 0.862 | 0.976 | 0.941 | 0.862 | 1 |
| FAM20A, RETN | | | | | | | | | |
| IFI27, OAS1, SIGLEC1, ISG15, SLC1A2, IL1R2, | 0.876 | 0.868 | 0.885 | 0.926 | 0.881 | 0.971 | 0.950 | 0.901 | 0.999 |
| MMP8 | | | | | | | | | |
| RSAD2, IFI27, IFIT1, SIGLEC1, ISG15, IL1R2, | 0.876 | 0.868 | 0.885 | 0.919 | 0.871 | 0.968 | 0.948 | 0.899 | 0.996 |
| MMP8 | | | | | | | | | |
| RSAD2, IFI27, IFI44L, HERC6, SLC1A2, | 0.876 | 0.867 | 0.886 | 0.923 | 0.868 | 0.978 | 0.930 | 0.848 | 1 |
| FAM20A, RETN | | | | | | | | | |
| RSAD2, IFI27, HERC6, SLC1A2, IL1R2, RETN, | 0.876 | 0.868 | 0.885 | 0.924 | 0.874 | 0.974 | 0.935 | 0.868 | 1 |
| MMP8 | | | | | | | | | |
| RSAD2, IFI27, OAS1, SIGLEC1, SLC1A2, IL1R2, | 0.876 | 0.868 | 0.885 | 0.924 | 0.878 | 0.971 | 0.953 | 0.903 | 1 |
| MMP8 | | | | | | | | | |
| IFI27, IFI44L, SIGLEC1, HERC6, SLC1A2, | 0.876 | 0.868 | 0.885 | 0.927 | 0.883 | 0.972 | 0.946 | 0.896 | 0.995 |
| IL1R2, MMP8 | | | | | | | | | |
| IFI27, IFI44L, SIGLEC1, ISG15, HERC6, | 0.876 | 0.868 | 0.885 | 0.926 | 0.881 | 0.970 | 0.943 | 0.892 | 0.995 |
| SLC1A2, IL1R2 | | | | | | | | | |
| RSAD2, IFI27, SIGLEC1, ISG15, SLC1A2, | 0.876 | 0.868 | 0.884 | 0.924 | 0.878 | 0.969 | 0.950 | 0.901 | 0.999 |
| IL1R2, MMP8 | | | | | | | | | |
| IFI44L, SIGLEC1, ISG15, HERC6, IL1R2, | 0.876 | 0.867 | 0.886 | 0.912 | 0.852 | 0.973 | 0.955 | 0.895 | 1 |
| FAM20A, RETN | | | | | | | | | |
| RSAD2, IFIT1, IFI44L, SIGLEC1, HERC6, | 0.876 | 0.867 | 0.886 | 0.921 | 0.869 | 0.973 | 0.933 | 0.856 | 1 |
| SLC1A2, FAM20A | | | | | | | | | |
| RSAD2, IFI27, OAS1, IFI44L, IL1R2, RETN, | 0.876 | 0.867 | 0.885 | 0.918 | 0.863 | 0.973 | 0.942 | 0.883 | 1 |
| MMP8 | | | | | | | | | |
| IFI27, IFI44L, HERC6, SLC1A2, IL1R2, RETN, | 0.876 | 0.867 | 0.885 | 0.924 | 0.874 | 0.973 | 0.935 | 0.868 | 1 |
| MMP8 | | | | | | | | | |
| IFI44L, SIGLEC1, ISG15, SLC1A2, IL1R2, | 0.876 | 0.867 | 0.885 | 0.917 | 0.863 | 0.971 | 0.954 | 0.893 | 1 |
| OLAH, FAM20A | | | | | | | | | |
| RSAD2, IFI27, OAS1, IFIT1, HERC6, SLC1A2, | 0.876 | 0.867 | 0.886 | 0.914 | 0.853 | 0.974 | 0.930 | 0.852 | 1 |
| RETN | | | | | | | | | |
| IFIT1, IFI44L, SIGLEC1, ISG15, OLAH, | 0.876 | 0.867 | 0.885 | 0.904 | 0.835 | 0.973 | 0.945 | 0.888 | 1 |
| FAM20A, RETN | | | | | | | | | |
| IFI27, IFIT1, SIGLEC1, ISG15, HERC6, SLC1A2, | 0.876 | 0.868 | 0.885 | 0.926 | 0.880 | 0.971 | 0.950 | 0.902 | 0.998 |
| IL1R2 | | | | | | | | | |
| RSAD2, IFI27, IFIT1, HERC6, SLC1A2, | 0.876 | 0.867 | 0.886 | 0.921 | 0.865 | 0.977 | 0.932 | 0.849 | 1 |
| FAM20A, RETN | | | | | | | | | |
| IFI27, OAS1, IFIT1, IFI44L, SIGLEC1, SLC1A2, | 0.876 | 0.868 | 0.885 | 0.924 | 0.878 | 0.970 | 0.946 | 0.894 | 0.998 |
| IL1R2 | | | | | | | | | |
| RSAD2, IFIT1, SIGLEC1, ISG15, IL1R2, | 0.876 | 0.867 | 0.885 | 0.911 | 0.848 | 0.973 | 0.964 | 0.916 | 1 |
| FAM20A, RETN | | | | | | | | | |
| OAS1, IFIT1, SIGLEC1, ISG15, OLAH, FAM20A, | 0.876 | 0.867 | 0.886 | 0.904 | 0.834 | 0.974 | 0.949 | 0.896 | 1 |
| RETN | | | | | | | | | |
| RSAD2, IFI27, OAS1, IFI44L, SIGLEC1, | 0.876 | 0.868 | 0.885 | 0.926 | 0.879 | 0.972 | 0.952 | 0.902 | 1 |
| SLC1A2, IL1R2 | | | | | | | | | |
| IFIT1, SIGLEC1, ISG15, SLC1A2, IL1R2, OLAH, | 0.876 | 0.867 | 0.885 | 0.917 | 0.862 | 0.972 | 0.950 | 0.890 | 1 |
| FAM20A | | | | | | | | | |
| RSAD2, OAS1, SIGLEC1, HERC6, IL1R2, | 0.876 | 0.866 | 0.886 | 0.912 | 0.852 | 0.971 | 0.958 | 0.902 | 1 |
| FAM20A, RETN | | | | | | | | | |
| IFI44L, SIGLEC1, HERC6, SLC1A2, OLAH, | 0.876 | 0.866 | 0.886 | 0.918 | 0.862 | 0.973 | 0.950 | 0.880 | 1 |
| FAM20A, MMP8 | | | | | | | | | |
| OAS1, IFIT1, IFI44L, SIGLEC1, OLAH, | 0.876 | 0.867 | 0.886 | 0.905 | 0.835 | 0.974 | 0.948 | 0.893 | 1 |
| FAM20A, RETN | | | | | | | | | |
| RSAD2, SIGLEC1, HERC6, SLC1A2, IL1R2, | 0.876 | 0.866 | 0.886 | 0.918 | 0.865 | 0.971 | 0.943 | 0.870 | 1 |
| OLAH, FAM20A | | | | | | | | | |
| RSAD2, IFI27, IFIT1, SIGLEC1, SLC1A2, IL1R2, | 0.876 | 0.868 | 0.884 | 0.925 | 0.879 | 0.970 | 0.953 | 0.906 | 1 |
| MMP8 | | | | | | | | | |
| IFI44L, SIGLEC1, ISG15, HERC6, SLC1A2, | 0.876 | 0.866 | 0.886 | 0.919 | 0.866 | 0.972 | 0.950 | 0.878 | 1 |
| IL1R2, FAM20A | | | | | | | | | |
| IFI44L, SIGLEC1, HERC6, SLC1A2, IL1R2, | 0.876 | 0.866 | 0.886 | 0.918 | 0.865 | 0.972 | 0.950 | 0.877 | 1 |
| OLAH, FAM20A | | | | | | | | | |
| RSAD2, IFI27, SIGLEC1, ISG15, HERC6, | 0.876 | 0.868 | 0.884 | 0.924 | 0.878 | 0.969 | 0.949 | 0.900 | 0.998 |
| SLC1A2, IL1R2 | | | | | | | | | |
| IFI44L, SIGLEC1, ISG15, IL1R2, OLAH, | 0.876 | 0.867 | 0.885 | 0.906 | 0.842 | 0.970 | 0.963 | 0.912 | 1 |
| FAM20A, RETN | | | | | | | | | |
| IFI27, OAS1, ISG15, HERC6, SLC1A2, RETN, | 0.876 | 0.866 | 0.885 | 0.915 | 0.858 | 0.972 | 0.930 | 0.857 | 1 |
| MMP8 | | | | | | | | | |
| OAS1, IFIT1, SIGLEC1, ISG15, SLC1A2, IL1R2, | 0.876 | 0.866 | 0.885 | 0.916 | 0.861 | 0.971 | 0.951 | 0.888 | 1 |
| FAM20A | | | | | | | | | |
| RSAD2, IFIT1, IFI44L, SIGLEC1, HERC6, | 0.876 | 0.867 | 0.885 | 0.910 | 0.848 | 0.971 | 0.932 | 0.860 | 1 |
| FAM20A, MMP8 | | | | | | | | | |
| IFI44L, SIGLEC1, HERC6, IL1R2, OLAH, | 0.876 | 0.866 | 0.885 | 0.912 | 0.851 | 0.973 | 0.957 | 0.896 | 1 |
| FAM20A, RETN | | | | | | | | | |
| OAS1, SIGLEC1, ISG15, HERC6, SLC1A2, | 0.876 | 0.866 | 0.886 | 0.919 | 0.869 | 0.970 | 0.949 | 0.878 | 1 |
| IL1R2, FAM20A | | | | | | | | | |
| RSAD2, IFI27, IFI44L, SIGLEC1, HERC6, | 0.876 | 0.867 | 0.884 | 0.926 | 0.880 | 0.972 | 0.948 | 0.900 | 0.996 |
| SLC1A2, IL1R2 | | | | | | | | | |
| IFI27, IFIT1, IFI44L, ISG15, HERC6, FAM20A, | 0.876 | 0.866 | 0.886 | 0.910 | 0.849 | 0.971 | 0.925 | 0.843 | 1 |
| MMP8 | | | | | | | | | |
| IFI27, IFIT1, IFI44L, SIGLEC1, HERC6, SLC1A2, | 0.876 | 0.867 | 0.884 | 0.926 | 0.881 | 0.971 | 0.947 | 0.897 | 0.996 |
| IL1R2 | | | | | | | | | |
| RSAD2, IFI27, OAS1, IFIT1, IFI44L, SIGLEC1, | 0.876 | 0.867 | 0.885 | 0.907 | 0.844 | 0.970 | 0.952 | 0.889 | 1 |
| RETN | | | | | | | | | |
| IFIT1, SIGLEC1, ISG15, OLAH, FAM20A, | 0.876 | 0.866 | 0.886 | 0.912 | 0.849 | 0.974 | 0.958 | 0.909 | 1 |
| RETN, MMP8 | | | | | | | | | |
| RSAD2, IFI27, IFIT1, HERC6, SLC1A2, IL1R2, | 0.876 | 0.867 | 0.884 | 0.922 | 0.872 | 0.972 | 0.936 | 0.872 | 1.000 |
| RETN | | | | | | | | | |
| SIGLEC1, ISG15, SLC1A2, OLAH, FAM20A, | 0.876 | 0.866 | 0.886 | 0.915 | 0.858 | 0.972 | 0.949 | 0.882 | 1 |
| RETN, MMP8 | | | | | | | | | |
| IFI27, OAS1, IFIT1, IFI44L, SIGLEC1, ISG15, | 0.876 | 0.867 | 0.885 | 0.909 | 0.848 | 0.970 | 0.955 | 0.897 | 1 |
| RETN | | | | | | | | | |
| OAS1, SIGLEC1, HERC6, SLC1A2, IL1R2, | 0.876 | 0.865 | 0.886 | 0.920 | 0.869 | 0.971 | 0.946 | 0.878 | 1 |
| OLAH, FAM20A | | | | | | | | | |
| OAS1, IFIT1, SIGLEC1, HERC6, SLC1A2, | 0.876 | 0.866 | 0.885 | 0.922 | 0.870 | 0.973 | 0.934 | 0.859 | 1 |
| FAM20A, RETN | | | | | | | | | |
| IFI27, IFIT1, HERC6, SLC1A2, IL1R2, RETN, | 0.876 | 0.867 | 0.884 | 0.925 | 0.875 | 0.974 | 0.943 | 0.885 | 1 |
| MMP8 | | | | | | | | | |
| IFI27, SIGLEC1, ISG15, HERC6, SLC1A2, | 0.876 | 0.867 | 0.884 | 0.924 | 0.879 | 0.970 | 0.950 | 0.901 | 0.999 |
| IL1R2, MMP8 | | | | | | | | | |
| OAS1, IFIT1, SIGLEC1, SLC1A2, OLAH, | 0.876 | 0.866 | 0.885 | 0.918 | 0.86 | 0.974 | 0.945 | 0.882 | 1 |
| FAM20A, RETN | | | | | | | | | |
| RSAD2, IFIT1, IFI44L, SIGLEC1, IL1R2, | 0.876 | 0.866 | 0.885 | 0.910 | 0.846 | 0.973 | 0.963 | 0.913 | 1 |
| FAM20A, RETN | | | | | | | | | |
| RSAD2, IFIT1, SIGLEC1, ISG15, SLC1A2, | 0.876 | 0.867 | 0.885 | 0.918 | 0.865 | 0.972 | 0.955 | 0.893 | 1 |
| IL1R2, FAM20A | | | | | | | | | |
| IFI44L, SIGLEC1, SLC1A2, IL1R2, OLAH, | 0.876 | 0.866 | 0.885 | 0.916 | 0.863 | 0.970 | 0.957 | 0.896 | 1 |
| FAM20A, RETN | | | | | | | | | |
| IFI27, OAS1, IFI44L, SIGLEC1, SLC1A2, IL1R2, | 0.876 | 0.867 | 0.884 | 0.924 | 0.878 | 0.970 | 0.945 | 0.893 | 0.996 |
| MMP8 | | | | | | | | | |
| RSAD2, IFIT1, IFI44L, SIGLEC1, SLC1A2, | 0.875 | 0.866 | 0.884 | 0.919 | 0.865 | 0.972 | 0.952 | 0.893 | 1 |
| IL1R2, FAM20A | | | | | | | | | |
| RSAD2, IFI27, OAS1, IFI44L, ISG15, IL1R2, | 0.875 | 0.867 | 0.884 | 0.918 | 0.864 | 0.971 | 0.941 | 0.883 | 1.000 |
| RETN | | | | | | | | | |
| RSAD2, OAS1, SIGLEC1, ISG15, IL1R2, OLAH, | 0.875 | 0.866 | 0.885 | 0.909 | 0.846 | 0.973 | 0.964 | 0.914 | 1 |
| FAM20A | | | | | | | | | |
| RSAD2, IFIT1, SIGLEC1, ISG15, SLC1A2, | 0.875 | 0.866 | 0.885 | 0.919 | 0.863 | 0.974 | 0.946 | 0.883 | 1 |
| OLAH, FAM20A | | | | | | | | | |
| IFI27, OAS1, IFIT1, SLC1A2, IL1R2, RETN, | 0.875 | 0.867 | 0.884 | 0.922 | 0.871 | 0.972 | 0.945 | 0.884 | 1 |
| MMP8 | | | | | | | | | |
| RSAD2, IFI27, IFI44L, HERC6, SLC1A2, IL1R2, | 0.875 | 0.867 | 0.884 | 0.924 | 0.874 | 0.973 | 0.933 | 0.866 | 0.999 |
| RETN | | | | | | | | | |
| RSAD2, IFI27, IFI44L, SIGLEC1, HERC6, | 0.875 | 0.867 | 0.884 | 0.921 | 0.868 | 0.975 | 0.939 | 0.880 | 0.998 |
| SLC1A2, RETN | | | | | | | | | |
| RSAD2, SIGLEC1, ISG15, HERC6, IL1R2, | 0.875 | 0.866 | 0.885 | 0.914 | 0.855 | 0.973 | 0.955 | 0.894 | 1 |
| FAM20A, RETN | | | | | | | | | |
| RSAD2, IFI27, ISG15, HERC6, SLC1A2, IL1R2, | 0.875 | 0.867 | 0.884 | 0.923 | 0.873 | 0.973 | 0.936 | 0.872 | 1.000 |
| RETN | | | | | | | | | |
| IFI27, OAS1, IFI44L, SIGLEC1, ISG15, SLC1A2, | 0.875 | 0.867 | 0.884 | 0.924 | 0.878 | 0.970 | 0.946 | 0.895 | 0.996 |
| IL1R2 | | | | | | | | | |
| IFI44L, SIGLEC1, SLC1A2, OLAH, FAM20A, | 0.875 | 0.866 | 0.885 | 0.916 | 0.858 | 0.974 | 0.949 | 0.880 | 1 |
| RETN, MMP8 | | | | | | | | | |
| RSAD2, IFI27, IFIT1, IFI44L, HERC6, FAM20A, | 0.875 | 0.866 | 0.885 | 0.913 | 0.853 | 0.973 | 0.921 | 0.838 | 1 |
| MMP8 | | | | | | | | | |
| IFI27, OAS1, IFIT1, HERC6, SLC1A2, IL1R2, | 0.875 | 0.866 | 0.884 | 0.921 | 0.875 | 0.967 | 0.940 | 0.879 | 1 |
| MMP8 | | | | | | | | | |
| RSAD2, OAS1, SIGLEC1, HERC6, OLAH, | 0.875 | 0.865 | 0.885 | 0.910 | 0.850 | 0.971 | 0.954 | 0.899 | 1 |
| FAM20A, MMP8 | | | | | | | | | |
| RSAD2, IFI27, IFIT1, ISG15, IL1R2, RETN, | 0.875 | 0.866 | 0.884 | 0.920 | 0.865 | 0.974 | 0.951 | 0.901 | 1 |
| MMP8 | | | | | | | | | |
| RSAD2, OAS1, IFI44L, SIGLEC1, IL1R2, OLAH, | 0.875 | 0.866 | 0.885 | 0.911 | 0.847 | 0.974 | 0.963 | 0.912 | 1 |
| FAM20A | | | | | | | | | |
| RSAD2, OAS1, IFIT1, SIGLEC1, SLC1A2, | 0.875 | 0.866 | 0.884 | 0.918 | 0.863 | 0.972 | 0.955 | 0.893 | 1 |
| IL1R2, FAM20A | | | | | | | | | |
| RSAD2, SIGLEC1, ISG15, HERC6, SLC1A2, | 0.875 | 0.865 | 0.885 | 0.918 | 0.866 | 0.970 | 0.948 | 0.875 | 1 |
| IL1R2, FAM20A | | | | | | | | | |
| IFI44L, SIGLEC1, ISG15, SLC1A2, IL1R2, | 0.875 | 0.866 | 0.884 | 0.915 | 0.862 | 0.968 | 0.953 | 0.885 | 1 |
| FAM20A, RETN | | | | | | | | | |
| RSAD2, IFI27, OAS1, IFI44L, SIGLEC1, | 0.875 | 0.865 | 0.885 | 0.918 | 0.859 | 0.976 | 0.947 | 0.875 | 1 |
| SLC1A2, FAM20A | | | | | | | | | |
| RSAD2, IFIT1, SIGLEC1, HERC6, SLC1A2, | 0.875 | 0.866 | 0.885 | 0.919 | 0.865 | 0.972 | 0.936 | 0.859 | 1 |
| FAM20A, RETN | | | | | | | | | |
| RSAD2, IFIT1, IFI44L, SIGLEC1, ISG15, OLAH, | 0.875 | 0.866 | 0.885 | 0.905 | 0.836 | 0.974 | 0.947 | 0.892 | 1 |
| FAM20A | | | | | | | | | |
| RSAD2, IFI27, HERC6, SLC1A2, FAM20A, | 0.875 | 0.865 | 0.885 | 0.923 | 0.868 | 0.979 | 0.935 | 0.853 | 1 |
| RETN, MMP8 | | | | | | | | | |
| IFI27, OAS1, IFI44L, ISG15, IL1R2, RETN, | 0.875 | 0.867 | 0.884 | 0.920 | 0.867 | 0.973 | 0.941 | 0.883 | 1.000 |
| MMP8 | | | | | | | | | |
| IFI27, IFIT1, SIGLEC1, ISG15, HERC6, SLC1A2, | 0.875 | 0.866 | 0.884 | 0.919 | 0.865 | 0.974 | 0.949 | 0.896 | 1 |
| RETN | | | | | | | | | |
| OAS1, IFIT1, SIGLEC1, SLC1A2, IL1R2, | 0.875 | 0.866 | 0.884 | 0.919 | 0.866 | 0.972 | 0.954 | 0.892 | 1 |
| FAM20A, RETN | | | | | | | | | |
| IFI27, OAS1, IFIT1, IFI44L, HERC6, SLC1A2, | 0.875 | 0.866 | 0.885 | 0.914 | 0.854 | 0.973 | 0.935 | 0.863 | 1 |
| RETN | | | | | | | | | |
| IFI27, IFIT1, IFI44L, ISG15, HERC6, RETN, | 0.875 | 0.866 | 0.885 | 0.910 | 0.848 | 0.971 | 0.936 | 0.871 | 1 |
| MMP8 | | | | | | | | | |
| IFI27, IFI44L, ISG15, HERC6, SLC1A2, IL1R2, | 0.875 | 0.867 | 0.884 | 0.923 | 0.874 | 0.973 | 0.933 | 0.866 | 0.999 |
| RETN | | | | | | | | | |
| RSAD2, IFI27, SIGLEC1, HERC6, SLC1A2, | 0.875 | 0.866 | 0.884 | 0.920 | 0.866 | 0.974 | 0.950 | 0.896 | 1 |
| RETN, MMP8 | | | | | | | | | |
| RSAD2, IFI27, SIGLEC1, ISG15, HERC6, | 0.875 | 0.866 | 0.884 | 0.919 | 0.865 | 0.974 | 0.945 | 0.887 | 1 |
| SLC1A2, RETN | | | | | | | | | |
| RSAD2, OAS1, SIGLEC1, IL1R2, OLAH, | 0.875 | 0.865 | 0.885 | 0.907 | 0.843 | 0.971 | 0.961 | 0.906 | 1 |
| FAM20A, RETN | | | | | | | | | |
| RSAD2, IFI27, IFI44L, ISG15, HERC6, RETN, | 0.875 | 0.866 | 0.884 | 0.914 | 0.855 | 0.973 | 0.940 | 0.883 | 0.998 |
| MMP8 | | | | | | | | | |
| RSAD2, IFI27, IFI44L, SIGLEC1, SLC1A2, | 0.875 | 0.867 | 0.883 | 0.925 | 0.879 | 0.972 | 0.951 | 0.905 | 0.997 |
| IL1R2, MMP8 | | | | | | | | | |
| RSAD2, SIGLEC1, ISG15, HERC6, FAM20A, | 0.875 | 0.866 | 0.884 | 0.913 | 0.853 | 0.974 | 0.951 | 0.888 | 1 |
| RETN, MMP8 | | | | | | | | | |
| OAS1, IFI44L, SIGLEC1, HERC6, IL1R2, | 0.875 | 0.866 | 0.884 | 0.914 | 0.854 | 0.973 | 0.961 | 0.908 | 1 |
| FAM20A, RETN | | | | | | | | | |
| OAS1, IFI44L, SIGLEC1, ISG15, IL1R2, OLAH, | 0.875 | 0.866 | 0.885 | 0.910 | 0.846 | 0.973 | 0.962 | 0.911 | 1 |
| FAM20A | | | | | | | | | |
| IFI27, IFIT1, IFI44L, HERC6, SLC1A2, IL1R2, | 0.875 | 0.866 | 0.884 | 0.923 | 0.873 | 0.973 | 0.933 | 0.866 | 0.999 |
| RETN | | | | | | | | | |
| OAS1, IFI44L, SIGLEC1, HERC6, SLC1A2, | 0.875 | 0.865 | 0.885 | 0.919 | 0.869 | 0.970 | 0.949 | 0.878 | 1 |
| IL1R2, FAM20A | | | | | | | | | |
| RSAD2, IFI27, OAS1, IFIT1, SIGLEC1, ISG15, | 0.875 | 0.866 | 0.884 | 0.909 | 0.847 | 0.971 | 0.952 | 0.892 | 1 |
| RETN | | | | | | | | | |
| IFIT1, IFI44L, SIGLEC1, ISG15, FAM20A, | 0.875 | 0.865 | 0.885 | 0.908 | 0.841 | 0.975 | 0.943 | 0.881 | 1 |
| RETN, MMP8 | | | | | | | | | |
| RSAD2, OAS1, SIGLEC1, HERC6, SLC1A2, | 0.875 | 0.865 | 0.885 | 0.919 | 0.868 | 0.969 | 0.951 | 0.881 | 1 |
| IL1R2, FAM20A | | | | | | | | | |
| RSAD2, IFI27, IFIT1, SIGLEC1, ISG15, SLC1A2, | 0.875 | 0.867 | 0.883 | 0.924 | 0.878 | 0.969 | 0.949 | 0.900 | 0.998 |
| IL1R2 | | | | | | | | | |
| RSAD2, IFI27, OAS1, IFIT1, HERC6, SLC1A2, | 0.875 | 0.866 | 0.884 | 0.922 | 0.875 | 0.968 | 0.938 | 0.875 | 1 |
| IL1R2 | | | | | | | | | |
| IFI27, IFIT1, HERC6, SLC1A2, FAM20A, RETN, | 0.875 | 0.865 | 0.885 | 0.920 | 0.863 | 0.977 | 0.936 | 0.855 | 1 |
| MMP8 | | | | | | | | | |
| RSAD2, IFIT1, SIGLEC1, ISG15, HERC6, | 0.875 | 0.866 | 0.884 | 0.918 | 0.864 | 0.971 | 0.938 | 0.862 | 1 |
| SLC1A2, FAM20A | | | | | | | | | |
| IFI27, IFIT1, SIGLEC1, HERC6, SLC1A2, RETN, | 0.875 | 0.866 | 0.884 | 0.919 | 0.865 | 0.974 | 0.940 | 0.880 | 1 |
| MMP8 | | | | | | | | | |
| IFIT1, SIGLEC1, ISG15, HERC6, SLC1A2, | 0.875 | 0.865 | 0.885 | 0.918 | 0.864 | 0.972 | 0.940 | 0.864 | 1 |
| FAM20A, MMP8 | | | | | | | | | |
| RSAD2, IFI27, IFI44L, SIGLEC1, ISG15, | 0.875 | 0.867 | 0.883 | 0.925 | 0.879 | 0.971 | 0.950 | 0.903 | 0.997 |
| SLC1A2, IL1R2 | | | | | | | | | |
| RSAD2, OAS1, IFIT1, SIGLEC1, OLAH, | 0.875 | 0.865 | 0.885 | 0.905 | 0.836 | 0.974 | 0.955 | 0.906 | 1 |
| FAM20A, RETN | | | | | | | | | |
| RSAD2, SIGLEC1, HERC6, OLAH, FAM20A, | 0.875 | 0.865 | 0.884 | 0.916 | 0.856 | 0.976 | 0.951 | 0.893 | 1 |
| RETN, MMP8 | | | | | | | | | |
| RSAD2, IFI27, IFIT1, ISG15, HERC6, RETN, | 0.875 | 0.865 | 0.884 | 0.914 | 0.855 | 0.973 | 0.937 | 0.877 | 0.997 |
| MMP8 | | | | | | | | | |
| OAS1, SIGLEC1, ISG15, HERC6, IL1R2, | 0.875 | 0.865 | 0.885 | 0.912 | 0.853 | 0.972 | 0.961 | 0.908 | 1 |
| FAM20A, RETN | | | | | | | | | |
| RSAD2, IFI27, IFI44L, ISG15, IL1R2, RETN, | 0.875 | 0.866 | 0.883 | 0.919 | 0.865 | 0.973 | 0.943 | 0.886 | 1 |
| MMP8 | | | | | | | | | |
| RSAD2, IFI27, SIGLEC1, ISG15, SLC1A2, | 0.875 | 0.865 | 0.885 | 0.917 | 0.858 | 0.976 | 0.946 | 0.872 | 1 |
| FAM20A, MMP8 | | | | | | | | | |
| RSAD2, IFI27, OAS1, IFIT1, SLC1A2, IL1R2, | 0.875 | 0.866 | 0.883 | 0.919 | 0.867 | 0.971 | 0.937 | 0.868 | 1 |
| RETN | | | | | | | | | |
| RSAD2, IFIT1, SIGLEC1, HERC6, SLC1A2, | 0.875 | 0.865 | 0.884 | 0.916 | 0.861 | 0.971 | 0.938 | 0.861 | 1 |
| FAM20A, MMP8 | | | | | | | | | |
| IFI27, OAS1, IFIT1, IFI44L, HERC6, FAM20A, | 0.875 | 0.865 | 0.885 | 0.912 | 0.853 | 0.971 | 0.920 | 0.837 | 1 |
| MMP8 | | | | | | | | | |
| SIGLEC1, ISG15, SLC1A2, IL1R2, OLAH, | 0.875 | 0.865 | 0.884 | 0.918 | 0.865 | 0.970 | 0.955 | 0.894 | 1 |
| FAM20A, RETN | | | | | | | | | |
| RSAD2, IFI27, OAS1, SIGLEC1, ISG15, SLC1A2, | 0.875 | 0.865 | 0.885 | 0.916 | 0.858 | 0.975 | 0.947 | 0.874 | 1 |
| FAM20A | | | | | | | | | |
| RSAD2, OAS1, IFIT1, SIGLEC1, HERC6, | 0.875 | 0.865 | 0.884 | 0.916 | 0.862 | 0.970 | 0.936 | 0.859 | 1 |
| SLC1A2, FAM20A | | | | | | | | | |
| IFI27, ISG15, HERC6, SLC1A2, IL1R2, RETN, | 0.875 | 0.866 | 0.884 | 0.925 | 0.876 | 0.975 | 0.936 | 0.870 | 1 |
| MMP8 | | | | | | | | | |
| SIGLEC1, HERC6, SLC1A2, OLAH, FAM20A, | 0.875 | 0.864 | 0.885 | 0.917 | 0.860 | 0.974 | 0.949 | 0.877 | 1 |
| RETN, MMP8 | | | | | | | | | |
| RSAD2, IFIT1, SIGLEC1, SLC1A2, IL1R2, | 0.875 | 0.865 | 0.884 | 0.920 | 0.868 | 0.972 | 0.957 | 0.895 | 1 |
| FAM20A, RETN | | | | | | | | | |
| RSAD2, SIGLEC1, HERC6, IL1R2, OLAH, | 0.875 | 0.865 | 0.885 | 0.912 | 0.852 | 0.972 | 0.955 | 0.897 | 1 |
| FAM20A, RETN | | | | | | | | | |
| RSAD2, SIGLEC1, SLC1A2, IL1R2, OLAH, | 0.875 | 0.865 | 0.884 | 0.916 | 0.862 | 0.970 | 0.955 | 0.894 | 1 |
| FAM20A, RETN | | | | | | | | | |
| RSAD2, IFIT1, SIGLEC1, OLAH, FAM20A, | 0.875 | 0.865 | 0.884 | 0.913 | 0.849 | 0.976 | 0.960 | 0.913 | 1 |
| RETN, MMP8 | | | | | | | | | |
| RSAD2, IFI27, OAS1, HERC6, SLC1A2, RETN, | 0.875 | 0.865 | 0.884 | 0.913 | 0.853 | 0.974 | 0.927 | 0.848 | 1 |
| MMP8 | | | | | | | | | |
| IFI27, IFI44L, SIGLEC1, ISG15, SLC1A2, | 0.875 | 0.865 | 0.884 | 0.915 | 0.856 | 0.974 | 0.943 | 0.866 | 1 |
| FAM20A, MMP8 | | | | | | | | | |
| OAS1, IFIT1, SIGLEC1, HERC6, SLC1A2, | 0.875 | 0.865 | 0.884 | 0.915 | 0.860 | 0.970 | 0.937 | 0.860 | 1 |
| FAM20A, MMP8 | | | | | | | | | |
| RSAD2, IFI27, OAS1, SIGLEC1, SLC1A2, | 0.875 | 0.864 | 0.885 | 0.919 | 0.860 | 0.977 | 0.949 | 0.878 | 1 |
| FAM20A, MMP8 | | | | | | | | | |
| OAS1, IFIT1, IFI44L, SIGLEC1, FAM20A, | 0.875 | 0.865 | 0.884 | 0.909 | 0.842 | 0.975 | 0.946 | 0.884 | 1 |
| RETN, MMP8 | | | | | | | | | |
| RSAD2, IFI27, OAS1, IFI44L, SIGLEC1, ISG15, | 0.875 | 0.865 | 0.884 | 0.909 | 0.846 | 0.971 | 0.953 | 0.891 | 1 |
| RETN | | | | | | | | | |
| RSAD2, IFI27, OAS1, ISG15, IL1R2, RETN, | 0.875 | 0.865 | 0.884 | 0.918 | 0.863 | 0.973 | 0.942 | 0.883 | 1 |
| MMP8 | | | | | | | | | |
| RSAD2, IFI27, IFI44L, SIGLEC1, ISG15, | 0.874 | 0.865 | 0.884 | 0.918 | 0.860 | 0.976 | 0.947 | 0.874 | 1 |
| SLC1A2, FAM20A | | | | | | | | | |
| IFI27, OAS1, IFIT1, ISG15, HERC6, SLC1A2, | 0.874 | 0.865 | 0.883 | 0.922 | 0.876 | 0.968 | 0.937 | 0.874 | 1 |
| IL1R2 | | | | | | | | | |
| RSAD2, IFI27, IFI44L, SIGLEC1, SLC1A2, | 0.874 | 0.864 | 0.884 | 0.918 | 0.859 | 0.978 | 0.947 | 0.873 | 1 |
| FAM20A, MMP8 | | | | | | | | | |
| SIGLEC1, ISG15, HERC6, IL1R2, OLAH, | 0.874 | 0.865 | 0.884 | 0.908 | 0.845 | 0.971 | 0.960 | 0.901 | 1 |
| FAM20A, RETN | | | | | | | | | |
| RSAD2, IFI27, OAS1, IFI44L, ISG15, FAM20A, | 0.874 | 0.864 | 0.884 | 0.910 | 0.846 | 0.974 | 0.945 | 0.869 | 1 |
| RETN | | | | | | | | | |
| RSAD2, IFIT1, SIGLEC1, SLC1A2, OLAH, | 0.874 | 0.865 | 0.884 | 0.919 | 0.863 | 0.975 | 0.950 | 0.890 | 1 |
| FAM20A, RETN | | | | | | | | | |
| IFI27, OAS1, IFIT1, SIGLEC1, ISG15, SLC1A2, | 0.874 | 0.866 | 0.883 | 0.915 | 0.859 | 0.971 | 0.947 | 0.881 | 1 |
| RETN | | | | | | | | | |
| IFI27, OAS1, SIGLEC1, ISG15, SLC1A2, | 0.874 | 0.864 | 0.885 | 0.916 | 0.857 | 0.975 | 0.946 | 0.872 | 1 |
| FAM20A, MMP8 | | | | | | | | | |
| RSAD2, IFI27, IFIT1, IFI44L, ISG15, HERC6, | 0.874 | 0.864 | 0.884 | 0.911 | 0.849 | 0.973 | 0.921 | 0.837 | 1 |
| FAM20A | | | | | | | | | |
| IFI27, IFI44L, SIGLEC1, ISG15, HERC6, | 0.874 | 0.865 | 0.884 | 0.916 | 0.859 | 0.973 | 0.943 | 0.866 | 1 |
| SLC1A2, FAM20A | | | | | | | | | |
| OAS1, IFIT1, SIGLEC1, ISG15, HERC6, | 0.874 | 0.865 | 0.884 | 0.918 | 0.865 | 0.971 | 0.938 | 0.862 | 1 |
| SLC1A2, FAM20A | | | | | | | | | |
| OAS1, SIGLEC1, SLC1A2, OLAH, FAM20A, | 0.874 | 0.864 | 0.884 | 0.915 | 0.859 | 0.972 | 0.951 | 0.891 | 1 |
| RETN, MMP8 | | | | | | | | | |
| SIGLEC1, ISG15, HERC6, SLC1A2, IL1R2, | 0.874 | 0.864 | 0.884 | 0.917 | 0.863 | 0.971 | 0.948 | 0.875 | 1 |
| OLAH, FAM20A | | | | | | | | | |
| IFI27, IFI44L, ISG15, SLC1A2, IL1R2, RETN, | 0.874 | 0.866 | 0.883 | 0.923 | 0.874 | 0.972 | 0.936 | 0.869 | 1 |
| MMP8 | | | | | | | | | |
| RSAD2, IFI27, OAS1, IFIT1, IFI44L, ISG15, | 0.874 | 0.866 | 0.883 | 0.916 | 0.864 | 0.969 | 0.941 | 0.888 | 0.995 |
| IL1R2 | | | | | | | | | |
| IFI27, OAS1, IFI44L, SIGLEC1, ISG15, SLC1A2, | 0.874 | 0.864 | 0.884 | 0.916 | 0.858 | 0.974 | 0.947 | 0.873 | 1 |
| FAM20A | | | | | | | | | |
| RSAD2, IFI44L, SIGLEC1, HERC6, SLC1A2, | 0.874 | 0.864 | 0.884 | 0.919 | 0.867 | 0.971 | 0.948 | 0.875 | 1 |
| IL1R2, FAM20A | | | | | | | | | |
| IFI44L, SIGLEC1, HERC6, OLAH, FAM20A, | 0.874 | 0.864 | 0.884 | 0.911 | 0.846 | 0.976 | 0.958 | 0.899 | 1 |
| RETN, MMP8 | | | | | | | | | |
| IFIT1, SIGLEC1, ISG15, SLC1A2, IL1R2, | 0.874 | 0.865 | 0.883 | 0.920 | 0.869 | 0.972 | 0.955 | 0.898 | 1 |
| FAM20A, RETN | | | | | | | | | |
| RSAD2, IFI27, IFIT1, IFI44L, HERC6, RETN, | 0.874 | 0.865 | 0.884 | 0.911 | 0.850 | 0.972 | 0.933 | 0.868 | 0.997 |
| MMP8 | | | | | | | | | |
| OAS1, IFIT1, SIGLEC1, ISG15, FAM20A, RETN, | 0.874 | 0.864 | 0.884 | 0.907 | 0.840 | 0.974 | 0.950 | 0.886 | 1 |
| MMP8 | | | | | | | | | |
| IFI27, IFIT1, IFI44L, SLC1A2, IL1R2, RETN, | 0.874 | 0.865 | 0.883 | 0.922 | 0.870 | 0.973 | 0.941 | 0.881 | 1 |
| MMP8 | | | | | | | | | |
| IFI27, OAS1, IFIT1, ISG15, SLC1A2, IL1R2, | 0.874 | 0.865 | 0.883 | 0.920 | 0.868 | 0.971 | 0.941 | 0.878 | 1 |
| RETN | | | | | | | | | |
| RSAD2, OAS1, SIGLEC1, HERC6, FAM20A, | 0.874 | 0.865 | 0.884 | 0.910 | 0.847 | 0.973 | 0.948 | 0.881 | 1 |
| RETN, MMP8 | | | | | | | | | |
| RSAD2, IFI27, IFIT1, SLC1A2, IL1R2, RETN, | 0.874 | 0.865 | 0.883 | 0.922 | 0.871 | 0.973 | 0.947 | 0.890 | 1 |
| MMP8 | | | | | | | | | |
| RSAD2, IFI27, IFI44L, ISG15, HERC6, FAM20A, | 0.874 | 0.864 | 0.885 | 0.913 | 0.852 | 0.974 | 0.925 | 0.844 | 1 |
| MMP8 | | | | | | | | | |
| RSAD2, IFI27, IFIT1, IFI44L, SIGLEC1, SLC1A2, | 0.874 | 0.866 | 0.882 | 0.925 | 0.878 | 0.971 | 0.951 | 0.905 | 0.997 |
| IL1R2 | | | | | | | | | |
| RSAD2, IFI44L, SIGLEC1, ISG15, IL1R2, OLAH, | 0.874 | 0.864 | 0.883 | 0.910 | 0.846 | 0.973 | 0.963 | 0.912 | 1 |
| FAM20A | | | | | | | | | |
| RSAD2, OAS1, SIGLEC1, SLC1A2, IL1R2, | 0.874 | 0.864 | 0.884 | 0.916 | 0.861 | 0.971 | 0.957 | 0.897 | 1 |
| OLAH, FAM20A | | | | | | | | | |
| IFI27, OAS1, IFIT1, HERC6, SLC1A2, RETN, | 0.874 | 0.864 | 0.883 | 0.913 | 0.854 | 0.972 | 0.930 | 0.857 | 1 |
| MMP8 | | | | | | | | | |
| IFIT1, SIGLEC1, ISG15, SLC1A2, OLAH, | 0.874 | 0.864 | 0.883 | 0.919 | 0.863 | 0.974 | 0.942 | 0.881 | 1 |
| FAM20A, RETN | | | | | | | | | |
| RSAD2, IFI44L, SIGLEC1, HERC6, IL1R2, | 0.874 | 0.864 | 0.884 | 0.914 | 0.855 | 0.973 | 0.954 | 0.893 | 1 |
| FAM20A, RETN | | | | | | | | | |
| IFI27, OAS1, IFI44L, HERC6, SLC1A2, RETN, | 0.874 | 0.865 | 0.883 | 0.914 | 0.855 | 0.972 | 0.929 | 0.855 | 1 |
| MMP8 | | | | | | | | | |
| RSAD2, IFI27, OAS1, HERC6, SLC1A2, IL1R2, | 0.874 | 0.865 | 0.883 | 0.922 | 0.876 | 0.969 | 0.932 | 0.865 | 0.998 |
| MMP8 | | | | | | | | | |
| RSAD2, SIGLEC1, SLC1A2, OLAH, FAM20A, | 0.874 | 0.864 | 0.884 | 0.917 | 0.862 | 0.972 | 0.951 | 0.891 | 1 |
| RETN, MMP8 | | | | | | | | | |
| IFI27, IFI44L, HERC6, SLC1A2, FAM20A, | 0.874 | 0.863 | 0.884 | 0.920 | 0.862 | 0.978 | 0.942 | 0.863 | 1 |
| RETN, MMP8 | | | | | | | | | |
| OAS1, SIGLEC1, HERC6, IL1R2, OLAH, | 0.874 | 0.864 | 0.884 | 0.911 | 0.851 | 0.971 | 0.960 | 0.907 | 1 |
| FAM20A, RETN | | | | | | | | | |
| RSAD2, IFI27, IFIT1, IFI44L, ISG15, IL1R2, | 0.874 | 0.865 | 0.882 | 0.916 | 0.864 | 0.969 | 0.937 | 0.881 | 0.993 |
| MMP8 | | | | | | | | | |
| SIGLEC1, ISG15, HERC6, OLAH, FAM20A, | 0.874 | 0.863 | 0.884 | 0.907 | 0.840 | 0.975 | 0.962 | 0.910 | 1 |
| RETN, MMP8 | | | | | | | | | |
| IFI27, ISG15, HERC6, SLC1A2, FAM20A, RETN, | 0.874 | 0.863 | 0.884 | 0.919 | 0.861 | 0.977 | 0.940 | 0.860 | 1 |
| MMP8 | | | | | | | | | |
| RSAD2, IFI27, OAS1, IFI44L, ISG15, HERC6, | 0.874 | 0.863 | 0.884 | 0.913 | 0.851 | 0.974 | 0.926 | 0.845 | 1 |
| FAM20A | | | | | | | | | |
| IFI27, OAS1, IFIT1, IFI44L, SLC1A2, IL1R2, | 0.874 | 0.865 | 0.882 | 0.920 | 0.868 | 0.972 | 0.940 | 0.877 | 1 |
| RETN | | | | | | | | | |
| IFI27, IFIT1, ISG15, HERC6, SLC1A2, IL1R2, | 0.874 | 0.865 | 0.882 | 0.922 | 0.872 | 0.972 | 0.936 | 0.872 | 1.000 |
| RETN | | | | | | | | | |
| OAS1, SIGLEC1, HERC6, OLAH, FAM20A, | 0.874 | 0.864 | 0.884 | 0.913 | 0.851 | 0.974 | 0.961 | 0.911 | 1 |
| RETN, MMP8 | | | | | | | | | |
| IFI27, OAS1, IFIT1, IFI44L, SIGLEC1, SLC1A2, | 0.874 | 0.865 | 0.882 | 0.914 | 0.858 | 0.971 | 0.949 | 0.883 | 1 |
| RETN | | | | | | | | | |
| RSAD2, SIGLEC1, HERC6, SLC1A2, IL1R2, | 0.874 | 0.863 | 0.884 | 0.919 | 0.868 | 0.970 | 0.946 | 0.873 | 1 |
| FAM20A, RETN | | | | | | | | | |
| IFI27, OAS1, IFI44L, SIGLEC1, SLC1A2, | 0.874 | 0.864 | 0.884 | 0.918 | 0.859 | 0.977 | 0.946 | 0.872 | 1 |
| FAM20A, MMP8 | | | | | | | | | |
| IFI44L, SIGLEC1, ISG15, HERC6, OLAH, | 0.874 | 0.863 | 0.884 | 0.905 | 0.840 | 0.971 | 0.959 | 0.904 | 1 |
| FAM20A, MMP8 | | | | | | | | | |
| IFI27, IFI44L, SIGLEC1, ISG15, HERC6, | 0.874 | 0.865 | 0.883 | 0.917 | 0.860 | 0.973 | 0.943 | 0.885 | 1 |
| SLC1A2, RETN | | | | | | | | | |
| OAS1, SIGLEC1, ISG15, IL1R2, OLAH, | 0.874 | 0.864 | 0.883 | 0.906 | 0.842 | 0.971 | 0.961 | 0.904 | 1 |
| FAM20A, RETN | | | | | | | | | |
| OAS1, SIGLEC1, ISG15, HERC6, FAM20A, | 0.874 | 0.864 | 0.883 | 0.911 | 0.848 | 0.974 | 0.952 | 0.889 | 1 |
| RETN, MMP8 | | | | | | | | | |
| IFI27, IFIT1, IFI44L, ISG15, SLC1A2, IL1R2, | 0.874 | 0.865 | 0.882 | 0.919 | 0.867 | 0.971 | 0.938 | 0.875 | 1 |
| RETN | | | | | | | | | |
| OAS1, SIGLEC1, ISG15, SLC1A2, IL1R2, OLAH, | 0.874 | 0.864 | 0.883 | 0.916 | 0.861 | 0.970 | 0.954 | 0.893 | 1 |
| FAM20A | | | | | | | | | |
| OAS1, IFI44L, SIGLEC1, IL1R2, OLAH, | 0.874 | 0.864 | 0.883 | 0.905 | 0.840 | 0.970 | 0.961 | 0.906 | 1 |
| FAM20A, RETN | | | | | | | | | |
| RSAD2, SIGLEC1, ISG15, SLC1A2, IL1R2, | 0.873 | 0.864 | 0.883 | 0.915 | 0.860 | 0.970 | 0.955 | 0.894 | 1 |
| OLAH, FAM20A | | | | | | | | | |
| IFI27, IFI44L, SIGLEC1, HERC6, SLC1A2, | 0.873 | 0.864 | 0.883 | 0.913 | 0.855 | 0.972 | 0.945 | 0.866 | 1 |
| FAM20A, MMP8 | | | | | | | | | |
| OAS1, SIGLEC1, SLC1A2, IL1R2, OLAH, | 0.873 | 0.864 | 0.883 | 0.916 | 0.862 | 0.970 | 0.957 | 0.897 | 1 |
| FAM20A, RETN | | | | | | | | | |
| IFI27, OAS1, IFIT1, IFI44L, ISG15, IL1R2, | 0.873 | 0.865 | 0.882 | 0.917 | 0.865 | 0.969 | 0.937 | 0.881 | 0.993 |
| MMP8 | | | | | | | | | |
| RSAD2, IFI27, OAS1, IFIT1, ISG15, IL1R2, | 0.873 | 0.865 | 0.882 | 0.914 | 0.862 | 0.965 | 0.945 | 0.892 | 0.997 |
| MMP8 | | | | | | | | | |
| IFI27, OAS1, IFIT1, SIGLEC1, ISG15, RETN, | 0.873 | 0.864 | 0.883 | 0.909 | 0.848 | 0.970 | 0.955 | 0.897 | 1 |
| MMP8 | | | | | | | | | |
| IFI27, IFIT1, ISG15, SLC1A2, IL1R2, RETN, | 0.873 | 0.864 | 0.882 | 0.922 | 0.871 | 0.973 | 0.946 | 0.887 | 1 |
| MMP8 | | | | | | | | | |
| IFI27, OAS1, IFIT1, IFI44L, SIGLEC1, RETN, | 0.873 | 0.864 | 0.883 | 0.911 | 0.850 | 0.972 | 0.954 | 0.896 | 1 |
| MMP8 | | | | | | | | | |
| RSAD2, SIGLEC1, ISG15, IL1R2, OLAH, | 0.873 | 0.864 | 0.883 | 0.906 | 0.841 | 0.970 | 0.963 | 0.910 | 1 |
| FAM20A, RETN | | | | | | | | | |
| IFI44L, ISG15, SLC1A2, IL1R2, OLAH, | 0.873 | 0.865 | 0.882 | 0.917 | 0.865 | 0.969 | 0.925 | 0.841 | 1 |
| FAM20A, MMP8 | | | | | | | | | |
| RSAD2, IFI27, OAS1, IFIT1, SIGLEC1, SLC1A2, | 0.873 | 0.865 | 0.882 | 0.915 | 0.856 | 0.973 | 0.949 | 0.880 | 1 |
| RETN | | | | | | | | | |
| IFI27, OAS1, IFIT1, SIGLEC1, SLC1A2, RETN, | 0.873 | 0.864 | 0.883 | 0.916 | 0.860 | 0.972 | 0.949 | 0.883 | 1 |
| MMP8 | | | | | | | | | |
| RSAD2, IFI27, OAS1, IFIT1, IFI44L, FAM20A, | 0.873 | 0.863 | 0.883 | 0.912 | 0.850 | 0.975 | 0.945 | 0.869 | 1 |
| RETN | | | | | | | | | |
| RSAD2, IFI27, IFIT1, IFI44L, ISG15, FAM20A, | 0.873 | 0.863 | 0.883 | 0.911 | 0.847 | 0.974 | 0.942 | 0.865 | 1 |
| RETN | | | | | | | | | |
| RSAD2, SIGLEC1, ISG15, HERC6, OLAH, | 0.873 | 0.863 | 0.883 | 0.910 | 0.850 | 0.970 | 0.953 | 0.898 | 1 |
| FAM20A, MMP8 | | | | | | | | | |
| RSAD2, OAS1, SIGLEC1, ISG15, IL1R2, | 0.873 | 0.863 | 0.883 | 0.907 | 0.843 | 0.971 | 0.961 | 0.904 | 1 |
| FAM20A, RETN | | | | | | | | | |
| OAS1, SIGLEC1, HERC6, SLC1A2, IL1R2, | 0.873 | 0.863 | 0.883 | 0.920 | 0.869 | 0.970 | 0.951 | 0.883 | 1 |
| FAM20A, RETN | | | | | | | | | |
| IFI27, OAS1, IFIT1, IFI44L, ISG15, FAM20A, | 0.873 | 0.863 | 0.883 | 0.910 | 0.847 | 0.973 | 0.945 | 0.868 | 1 |
| RETN | | | | | | | | | |
| RSAD2, IFI27, OAS1, ISG15, HERC6, SLC1A2, | 0.873 | 0.865 | 0.882 | 0.924 | 0.879 | 0.969 | 0.933 | 0.867 | 0.998 |
| IL1R2 | | | | | | | | | |
| RSAD2, OAS1, IFI44L, SIGLEC1, ISG15, IL1R2, | 0.873 | 0.863 | 0.883 | 0.910 | 0.846 | 0.973 | 0.963 | 0.912 | 1 |
| FAM20A | | | | | | | | | |
| RSAD2, IFI27, IFIT1, IFI44L, SIGLEC1, ISG15, | 0.873 | 0.864 | 0.882 | 0.909 | 0.848 | 0.970 | 0.950 | 0.892 | 1 |
| RETN | | | | | | | | | |
| IFIT1, IFI44L, SIGLEC1, ISG15, SLC1A2, | 0.873 | 0.864 | 0.882 | 0.911 | 0.851 | 0.970 | 0.938 | 0.864 | 1 |
| FAM20A, MMP8 | | | | | | | | | |
| OAS1, SIGLEC1, ISG15, HERC6, SLC1A2, | 0.873 | 0.863 | 0.883 | 0.915 | 0.860 | 0.970 | 0.943 | 0.874 | 1 |
| OLAH, FAM20A | | | | | | | | | |
| OAS1, IFI44L, SIGLEC1, ISG15, IL1R2, | 0.873 | 0.863 | 0.883 | 0.908 | 0.844 | 0.971 | 0.960 | 0.903 | 1 |
| FAM20A, RETN | | | | | | | | | |
| IFI27, IFIT1, IFI44L, ISG15, FAM20A, RETN, | 0.873 | 0.862 | 0.884 | 0.912 | 0.849 | 0.975 | 0.948 | 0.874 | 1 |
| MMP8 | | | | | | | | | |
| IFI44L, SIGLEC1, ISG15, HERC6, SLC1A2, | 0.873 | 0.863 | 0.883 | 0.914 | 0.858 | 0.971 | 0.946 | 0.872 | 1 |
| OLAH, FAM20A | | | | | | | | | |
| RSAD2, IFI27, IFIT1, HERC6, SLC1A2, IL1R2, | 0.873 | 0.864 | 0.882 | 0.923 | 0.877 | 0.968 | 0.937 | 0.877 | 0.997 |
| MMP8 | | | | | | | | | |
| RSAD2, OAS1, SIGLEC1, HERC6, SLC1A2, | 0.873 | 0.862 | 0.883 | 0.916 | 0.862 | 0.971 | 0.943 | 0.875 | 1 |
| OLAH, FAM20A | | | | | | | | | |
| OAS1, IFIT1, IFI44L, SIGLEC1, ISG15, | 0.873 | 0.863 | 0.883 | 0.902 | 0.832 | 0.972 | 0.942 | 0.881 | 1 |
| FAM20A, RETN | | | | | | | | | |
| IFI27, OAS1, IFIT1, IFI44L, HERC6, SLC1A2, | 0.873 | 0.864 | 0.882 | 0.922 | 0.876 | 0.968 | 0.936 | 0.874 | 0.998 |
| IL1R2 | | | | | | | | | |
| IFI44L, SIGLEC1, ISG15, SLC1A2, OLAH, | 0.873 | 0.863 | 0.882 | 0.914 | 0.856 | 0.971 | 0.946 | 0.879 | 1 |
| FAM20A, RETN | | | | | | | | | |
| RSAD2, IFI27, OAS1, IFIT1, SIGLEC1, RETN, | 0.873 | 0.863 | 0.883 | 0.910 | 0.848 | 0.972 | 0.954 | 0.895 | 1 |
| MMP8 | | | | | | | | | |
| RSAD2, SIGLEC1, ISG15, SLC1A2, IL1R2, | 0.873 | 0.863 | 0.883 | 0.915 | 0.862 | 0.968 | 0.952 | 0.882 | 1 |
| FAM20A, RETN | | | | | | | | | |
| RSAD2, SIGLEC1, HERC6, SLC1A2, FAM20A, | 0.873 | 0.863 | 0.883 | 0.912 | 0.854 | 0.971 | 0.949 | 0.877 | 1 |
| RETN, MMP8 | | | | | | | | | |
| IFI44L, SIGLEC1, HERC6, SLC1A2, IL1R2, | 0.873 | 0.863 | 0.883 | 0.920 | 0.868 | 0.971 | 0.952 | 0.880 | 1 |
| FAM20A, RETN | | | | | | | | | |
| RSAD2, IFIT1, IFI44L, SIGLEC1, OLAH, | 0.873 | 0.863 | 0.882 | 0.905 | 0.836 | 0.974 | 0.947 | 0.892 | 1 |
| FAM20A, RETN | | | | | | | | | |
| RSAD2, IFI27, OAS1, SLC1A2, IL1R2, RETN, | 0.873 | 0.864 | 0.881 | 0.924 | 0.874 | 0.974 | 0.934 | 0.863 | 1 |
| MMP8 | | | | | | | | | |
| RSAD2, OAS1, IFIT1, SIGLEC1, FAM20A, | 0.873 | 0.863 | 0.882 | 0.909 | 0.843 | 0.974 | 0.952 | 0.889 | 1 |
| RETN, MMP8 | | | | | | | | | |
| RSAD2, OAS1, SIGLEC1, ISG15, OLAH, | 0.873 | 0.862 | 0.883 | 0.905 | 0.840 | 0.971 | 0.960 | 0.910 | 1 |
| FAM20A, MMP8 | | | | | | | | | |
| SIGLEC1, HERC6, SLC1A2, IL1R2, OLAH, | 0.873 | 0.862 | 0.883 | 0.918 | 0.864 | 0.971 | 0.947 | 0.874 | 1 |
| FAM20A, RETN | | | | | | | | | |
| RSAD2, SIGLEC1, ISG15, HERC6, SLC1A2, | 0.872 | 0.862 | 0.883 | 0.915 | 0.860 | 0.970 | 0.941 | 0.871 | 1 |
| OLAH, FAM20A | | | | | | | | | |
| RSAD2, IFI27, OAS1, IFI44L, HERC6, SLC1A2, | 0.872 | 0.863 | 0.881 | 0.923 | 0.878 | 0.968 | 0.930 | 0.864 | 0.997 |
| IL1R2 | | | | | | | | | |
| RSAD2, IFI44L, SIGLEC1, SLC1A2, IL1R2, | 0.872 | 0.862 | 0.882 | 0.916 | 0.861 | 0.970 | 0.954 | 0.893 | 1 |
| OLAH, FAM20A | | | | | | | | | |
| IFI27, OAS1, IFI44L, SIGLEC1, ISG15, SLC1A2, | 0.872 | 0.864 | 0.881 | 0.915 | 0.858 | 0.971 | 0.948 | 0.882 | 1 |
| RETN | | | | | | | | | |
| RSAD2, IFI27, OAS1, IFI44L, SIGLEC1, | 0.872 | 0.864 | 0.881 | 0.916 | 0.858 | 0.974 | 0.948 | 0.876 | 1 |
| SLC1A2, RETN | | | | | | | | | |
| RSAD2, OAS1, IFI44L, SIGLEC1, OLAH, | 0.872 | 0.862 | 0.882 | 0.906 | 0.840 | 0.972 | 0.958 | 0.910 | 1 |
| FAM20A, MMP8 | | | | | | | | | |
| IFI27, OAS1, ISG15, HERC6, SLC1A2, IL1R2, | 0.872 | 0.864 | 0.881 | 0.923 | 0.878 | 0.968 | 0.927 | 0.860 | 0.994 |
| MMP8 | | | | | | | | | |
| RSAD2, IFI27, OAS1, IFIT1, IFI44L, HERC6, | 0.872 | 0.862 | 0.882 | 0.910 | 0.848 | 0.972 | 0.926 | 0.845 | 1 |
| FAM20A | | | | | | | | | |
| RSAD2, IFIT1, SIGLEC1, ISG15, OLAH, | 0.872 | 0.863 | 0.882 | 0.904 | 0.834 | 0.974 | 0.953 | 0.902 | 1 |
| FAM20A, RETN | | | | | | | | | |
| RSAD2, OAS1, SIGLEC1, OLAH, FAM20A, | 0.872 | 0.862 | 0.882 | 0.908 | 0.843 | 0.973 | 0.966 | 0.924 | 1 |
| RETN, MMP8 | | | | | | | | | |
| OAS1, IFI44L, SIGLEC1, SLC1A2, IL1R2, | 0.872 | 0.863 | 0.882 | 0.916 | 0.862 | 0.970 | 0.954 | 0.895 | 1 |
| OLAH, FAM20A | | | | | | | | | |
| RSAD2, IFI27, OAS1, IFI44L, HERC6, FAM20A, | 0.872 | 0.862 | 0.883 | 0.911 | 0.851 | 0.971 | 0.925 | 0.844 | 1 |
| MMP8 | | | | | | | | | |
| IFI27, SIGLEC1, ISG15, HERC6, SLC1A2, | 0.872 | 0.862 | 0.883 | 0.912 | 0.852 | 0.972 | 0.943 | 0.866 | 1 |
| FAM20A, MMP8 | | | | | | | | | |
| IFI27, OAS1, IFI44L, SIGLEC1, ISG15, RETN, | 0.872 | 0.862 | 0.882 | 0.911 | 0.850 | 0.972 | 0.953 | 0.893 | 1 |
| MMP8 | | | | | | | | | |
| IFI44L, SIGLEC1, ISG15, OLAH, FAM20A, | 0.872 | 0.862 | 0.882 | 0.907 | 0.840 | 0.973 | 0.967 | 0.925 | 1 |
| RETN, MMP8 | | | | | | | | | |
| RSAD2, IFI44L, SIGLEC1, IL1R2, OLAH, | 0.872 | 0.862 | 0.882 | 0.906 | 0.842 | 0.970 | 0.963 | 0.912 | 1 |
| FAM20A, RETN | | | | | | | | | |
| IFIT1, IFI44L, SIGLEC1, SLC1A2, FAM20A, | 0.872 | 0.863 | 0.882 | 0.918 | 0.861 | 0.974 | 0.939 | 0.868 | 1 |
| RETN, MMP8 | | | | | | | | | |
| RSAD2, OAS1, IFIT1, IFI44L, SIGLEC1, ISG15, | 0.872 | 0.863 | 0.881 | 0.902 | 0.832 | 0.973 | 0.939 | 0.873 | 1 |
| FAM20A | | | | | | | | | |
| RSAD2, IFI27, OAS1, IFI44L, SLC1A2, IL1R2, | 0.872 | 0.864 | 0.880 | 0.920 | 0.868 | 0.972 | 0.929 | 0.854 | 1 |
| RETN | | | | | | | | | |
| OAS1, IFI44L, SIGLEC1, HERC6, SLC1A2, | 0.872 | 0.862 | 0.882 | 0.916 | 0.861 | 0.971 | 0.941 | 0.872 | 1 |
| OLAH, FAM20A | | | | | | | | | |
| RSAD2, IFI27, IFIT1, ISG15, HERC6, SLC1A2, | 0.872 | 0.864 | 0.880 | 0.923 | 0.878 | 0.968 | 0.937 | 0.877 | 0.997 |
| IL1R2 | | | | | | | | | |
| RSAD2, OAS1, HERC6, IL1R2, OLAH, FAM20A, | 0.872 | 0.862 | 0.882 | 0.908 | 0.849 | 0.968 | 0.936 | 0.858 | 1 |
| MMP8 | | | | | | | | | |
| RSAD2, OAS1, IFIT1, SIGLEC1, ISG15, | 0.872 | 0.862 | 0.882 | 0.902 | 0.833 | 0.972 | 0.950 | 0.891 | 1 |
| FAM20A, RETN | | | | | | | | | |
| OAS1, IFI44L, SIGLEC1, ISG15, SLC1A2, IL1R2, | 0.872 | 0.862 | 0.881 | 0.915 | 0.860 | 0.969 | 0.953 | 0.890 | 1 |
| FAM20A | | | | | | | | | |
| RSAD2, IFI27, OAS1, IFIT1, SLC1A2, IL1R2, | 0.872 | 0.863 | 0.881 | 0.920 | 0.872 | 0.968 | 0.936 | 0.871 | 1 |
| MMP8 | | | | | | | | | |
| RSAD2, OAS1, IFI44L, SIGLEC1, IL1R2, | 0.872 | 0.862 | 0.882 | 0.906 | 0.842 | 0.971 | 0.961 | 0.906 | 1 |
| FAM20A, RETN | | | | | | | | | |
| RSAD2, IFI27, ISG15, SLC1A2, IL1R2, RETN, | 0.872 | 0.863 | 0.880 | 0.922 | 0.872 | 0.972 | 0.930 | 0.860 | 1 |
| MMP8 | | | | | | | | | |
| RSAD2, IFI27, OAS1, IFI44L, FAM20A, RETN, | 0.872 | 0.861 | 0.883 | 0.913 | 0.850 | 0.976 | 0.946 | 0.872 | 1 |
| MMP8 | | | | | | | | | |
| OAS1, IFIT1, SIGLEC1, HERC6, IL1R2, RETN, | 0.872 | 0.862 | 0.881 | 0.904 | 0.842 | 0.967 | 0.963 | 0.925 | 1 |
| MMP8 | | | | | | | | | |
| RSAD2, IFI27, IFIT1, ISG15, SLC1A2, IL1R2, | 0.872 | 0.863 | 0.880 | 0.921 | 0.870 | 0.972 | 0.936 | 0.872 | 0.999 |
| RETN | | | | | | | | | |
| IFI27, OAS1, IFIT1, ISG15, HERC6, FAM20A, | 0.872 | 0.862 | 0.882 | 0.912 | 0.854 | 0.969 | 0.925 | 0.844 | 1 |
| MMP8 | | | | | | | | | |
| RSAD2, IFI44L, SIGLEC1, SLC1A2, IL1R2, | 0.872 | 0.862 | 0.881 | 0.915 | 0.862 | 0.969 | 0.954 | 0.888 | 1 |
| FAM20A, RETN | | | | | | | | | |
| OAS1, IFI44L, SIGLEC1, ISG15, SLC1A2, | 0.872 | 0.862 | 0.882 | 0.912 | 0.854 | 0.971 | 0.947 | 0.885 | 1 |
| OLAH, FAM20A | | | | | | | | | |
| RSAD2, IFI27, OAS1, IFIT1, ISG15, FAM20A, | 0.872 | 0.862 | 0.882 | 0.911 | 0.850 | 0.971 | 0.943 | 0.867 | 1 |
| RETN | | | | | | | | | |
| RSAD2, IFI27, IFIT1, IFI44L, SLC1A2, IL1R2, | 0.872 | 0.863 | 0.880 | 0.919 | 0.867 | 0.972 | 0.936 | 0.871 | 1 |
| RETN | | | | | | | | | |
| IFI27, IFIT1, IFI44L, SIGLEC1, ISG15, RETN, | 0.872 | 0.862 | 0.882 | 0.910 | 0.848 | 0.971 | 0.950 | 0.892 | 1 |
| MMP8 | | | | | | | | | |
| RSAD2, OAS1, SIGLEC1, ISG15, SLC1A2, | 0.872 | 0.862 | 0.882 | 0.915 | 0.861 | 0.969 | 0.954 | 0.891 | 1 |
| IL1R2, FAM20A | | | | | | | | | |
| RSAD2, IFI27, IFIT1, ISG15, SLC1A2, IL1R2, | 0.872 | 0.863 | 0.880 | 0.922 | 0.876 | 0.968 | 0.936 | 0.875 | 0.997 |
| MMP8 | | | | | | | | | |
| SIGLEC1, ISG15, HERC6, SLC1A2, IL1R2, | 0.872 | 0.861 | 0.882 | 0.918 | 0.866 | 0.970 | 0.950 | 0.878 | 1 |
| FAM20A, RETN | | | | | | | | | |
| RSAD2, IFI27, IFI44L, SLC1A2, IL1R2, RETN, | 0.872 | 0.863 | 0.880 | 0.923 | 0.874 | 0.973 | 0.930 | 0.860 | 1 |
| MMP8 | | | | | | | | | |
| RSAD2, IFI27, OAS1, IFI44L, ISG15, IL1R2, | 0.872 | 0.863 | 0.880 | 0.914 | 0.862 | 0.966 | 0.937 | 0.881 | 0.993 |
| MMP8 | | | | | | | | | |
| RSAD2, IFI27, OAS1, SIGLEC1, ISG15, SLC1A2, | 0.872 | 0.862 | 0.881 | 0.914 | 0.855 | 0.972 | 0.950 | 0.881 | 1 |
| RETN | | | | | | | | | |
| IFI27, OAS1, IFI44L, ISG15, HERC6, FAM20A, | 0.872 | 0.861 | 0.882 | 0.909 | 0.848 | 0.970 | 0.930 | 0.852 | 1 |
| MMP8 | | | | | | | | | |
| OAS1, IFIT1, IFI44L, SIGLEC1, ISG15, | 0.872 | 0.861 | 0.882 | 0.901 | 0.831 | 0.972 | 0.939 | 0.870 | 1 |
| FAM20A, MMP8 | | | | | | | | | |
| IFI27, IFI44L, SIGLEC1, HERC6, SLC1A2, | 0.872 | 0.862 | 0.881 | 0.916 | 0.858 | 0.973 | 0.943 | 0.885 | 1 |
| RETN, MMP8 | | | | | | | | | |
| RSAD2, IFI27, ISG15, HERC6, SLC1A2, IL1R2, | 0.872 | 0.863 | 0.880 | 0.921 | 0.875 | 0.967 | 0.928 | 0.862 | 0.995 |
| MMP8 | | | | | | | | | |
| IFI44L, ISG15, HERC6, IL1R2, OLAH, FAM20A, | 0.872 | 0.862 | 0.881 | 0.909 | 0.849 | 0.968 | 0.940 | 0.865 | 1 |
| MMP8 | | | | | | | | | |
| RSAD2, OAS1, SIGLEC1, ISG15, HERC6, | 0.872 | 0.862 | 0.881 | 0.911 | 0.851 | 0.972 | 0.946 | 0.879 | 1 |
| FAM20A, RETN | | | | | | | | | |
| OAS1, IFIT1, IFI44L, SIGLEC1, SLC1A2, | 0.872 | 0.862 | 0.881 | 0.911 | 0.852 | 0.970 | 0.937 | 0.862 | 1 |
| FAM20A, MMP8 | | | | | | | | | |
| IFI27, OAS1, IFIT1, ISG15, SLC1A2, IL1R2, | 0.871 | 0.863 | 0.880 | 0.921 | 0.874 | 0.968 | 0.936 | 0.874 | 0.998 |
| MMP8 | | | | | | | | | |
| IFI27, IFIT1, IFI44L, ISG15, SLC1A2, IL1R2, | 0.871 | 0.863 | 0.880 | 0.922 | 0.875 | 0.968 | 0.937 | 0.878 | 0.996 |
| MMP8 | | | | | | | | | |
| RSAD2, IFI27, IFIT1, ISG15, HERC6, FAM20A, | 0.871 | 0.861 | 0.881 | 0.912 | 0.853 | 0.970 | 0.925 | 0.844 | 1 |
| MMP8 | | | | | | | | | |
| RSAD2, IFI27, OAS1, IFIT1, ISG15, SLC1A2, | 0.871 | 0.863 | 0.880 | 0.921 | 0.874 | 0.968 | 0.937 | 0.872 | 1 |
| IL1R2 | | | | | | | | | |
| IFI44L, HERC6, SLC1A2, IL1R2, OLAH, | 0.871 | 0.862 | 0.881 | 0.918 | 0.867 | 0.969 | 0.930 | 0.849 | 1 |
| FAM20A, MMP8 | | | | | | | | | |
| RSAD2, IFI44L, SIGLEC1, HERC6, SLC1A2, | 0.871 | 0.861 | 0.882 | 0.917 | 0.862 | 0.972 | 0.939 | 0.868 | 1 |
| OLAH, FAM20A | | | | | | | | | |
| RSAD2, IFI27, OAS1, ISG15, SLC1A2, IL1R2, | 0.871 | 0.863 | 0.880 | 0.920 | 0.868 | 0.973 | 0.930 | 0.856 | 1 |
| RETN | | | | | | | | | |
| RSAD2, OAS1, SIGLEC1, SLC1A2, IL1R2, | 0.871 | 0.862 | 0.881 | 0.915 | 0.861 | 0.969 | 0.953 | 0.888 | 1 |
| FAM20A, RETN | | | | | | | | | |
| IFI27, OAS1, SIGLEC1, ISG15, SLC1A2, RETN, | 0.871 | 0.862 | 0.881 | 0.915 | 0.857 | 0.972 | 0.948 | 0.880 | 1 |
| MMP8 | | | | | | | | | |
| RSAD2, IFI27, IFIT1, IFI44L, HERC6, SLC1A2, | 0.871 | 0.863 | 0.880 | 0.923 | 0.877 | 0.969 | 0.936 | 0.876 | 0.995 |
| IL1R2 | | | | | | | | | |
| RSAD2, IFI27, IFI44L, ISG15, SLC1A2, IL1R2, | 0.871 | 0.863 | 0.879 | 0.921 | 0.870 | 0.972 | 0.928 | 0.857 | 0.999 |
| RETN | | | | | | | | | |
| RSAD2, IFI44L, SIGLEC1, HERC6, OLAH, | 0.871 | 0.861 | 0.881 | 0.910 | 0.850 | 0.970 | 0.953 | 0.898 | 1 |
| FAM20A, MMP8 | | | | | | | | | |
| RSAD2, IFI44L, SIGLEC1, ISG15, IL1R2, | 0.871 | 0.861 | 0.881 | 0.906 | 0.842 | 0.970 | 0.963 | 0.910 | 1 |
| FAM20A, RETN | | | | | | | | | |
| RSAD2, IFI27, OAS1, IFIT1, IFI44L, SLC1A2, | 0.871 | 0.863 | 0.880 | 0.921 | 0.874 | 0.969 | 0.940 | 0.879 | 1 |
| IL1R2 | | | | | | | | | |
| IFI27, OAS1, IFIT1, SIGLEC1, ISG15, HERC6, | 0.871 | 0.863 | 0.880 | 0.908 | 0.855 | 0.962 | 0.912 | 0.839 | 0.985 |
| MMP8 | | | | | | | | | |
| RSAD2, IFI27, OAS1, IFI44L, SIGLEC1, RETN, | 0.871 | 0.861 | 0.881 | 0.910 | 0.848 | 0.972 | 0.953 | 0.890 | 1 |
| MMP8 | | | | | | | | | |
| RSAD2, OAS1, IFIT1, IFI44L, SIGLEC1, | 0.871 | 0.861 | 0.881 | 0.904 | 0.834 | 0.973 | 0.939 | 0.870 | 1 |
| FAM20A, MMP8 | | | | | | | | | |
| RSAD2, IFI27, IFIT1, IFI44L, FAM20A, RETN, | 0.871 | 0.860 | 0.882 | 0.914 | 0.851 | 0.977 | 0.943 | 0.868 | 1 |
| MMP8 | | | | | | | | | |
| OAS1, SIGLEC1, ISG15, SLC1A2, IL1R2, | 0.871 | 0.861 | 0.881 | 0.915 | 0.861 | 0.968 | 0.952 | 0.884 | 1 |
| FAM20A, RETN | | | | | | | | | |
| RSAD2, IFI27, OAS1, SIGLEC1, ISG15, RETN, | 0.871 | 0.861 | 0.881 | 0.913 | 0.852 | 0.974 | 0.957 | 0.895 | 1 |
| MMP8 | | | | | | | | | |
| RSAD2, OAS1, SIGLEC1, ISG15, SLC1A2, | 0.871 | 0.861 | 0.881 | 0.912 | 0.854 | 0.971 | 0.950 | 0.891 | 1 |
| OLAH, FAM20A | | | | | | | | | |
| IFI27, IFIT1, IFI44L, HERC6, SLC1A2, IL1R2, | 0.871 | 0.862 | 0.880 | 0.923 | 0.878 | 0.969 | 0.935 | 0.875 | 0.995 |
| MMP8 | | | | | | | | | |
| OAS1, IFI44L, SIGLEC1, SLC1A2, IL1R2, | 0.871 | 0.862 | 0.881 | 0.915 | 0.861 | 0.969 | 0.955 | 0.893 | 1 |
| FAM20A, RETN | | | | | | | | | |
| IFI27, OAS1, IFIT1, IFI44L, FAM20A, RETN, | 0.871 | 0.860 | 0.882 | 0.913 | 0.851 | 0.976 | 0.947 | 0.871 | 1 |
| MMP8 | | | | | | | | | |
| RSAD2, IFI44L, SIGLEC1, ISG15, SLC1A2, | 0.871 | 0.862 | 0.881 | 0.914 | 0.860 | 0.969 | 0.951 | 0.882 | 1 |
| IL1R2, FAM20A | | | | | | | | | |
| IFI44L, SIGLEC1, ISG15, HERC6, FAM20A, | 0.871 | 0.861 | 0.881 | 0.907 | 0.840 | 0.974 | 0.955 | 0.888 | 1 |
| RETN, MMP8 | | | | | | | | | |
| OAS1, SIGLEC1, HERC6, SLC1A2, FAM20A, | 0.871 | 0.861 | 0.881 | 0.912 | 0.854 | 0.971 | 0.950 | 0.881 | 1 |
| RETN, MMP8 | | | | | | | | | |
| RSAD2, IFI27, OAS1, ISG15, HERC6, FAM20A, | 0.871 | 0.861 | 0.881 | 0.912 | 0.854 | 0.970 | 0.927 | 0.847 | 1 |
| MMP8 | | | | | | | | | |
| IFI27, OAS1, IFIT1, IFI44L, SLC1A2, IL1R2, | 0.871 | 0.862 | 0.880 | 0.919 | 0.872 | 0.967 | 0.937 | 0.877 | 0.997 |
| MMP8 | | | | | | | | | |
| RSAD2, IFI27, OAS1, SIGLEC1, SLC1A2, RETN, | 0.871 | 0.861 | 0.881 | 0.914 | 0.855 | 0.973 | 0.950 | 0.881 | 1 |
| MMP8 | | | | | | | | | |
| IFI27, OAS1, IFI44L, SLC1A2, IL1R2, RETN, | 0.871 | 0.862 | 0.880 | 0.924 | 0.875 | 0.973 | 0.932 | 0.862 | 1 |
| MMP8 | | | | | | | | | |
| RSAD2, OAS1, IFI44L, SIGLEC1, SLC1A2, | 0.871 | 0.861 | 0.880 | 0.915 | 0.860 | 0.970 | 0.955 | 0.894 | 1 |
| IL1R2, FAM20A | | | | | | | | | |
| IFIT1, IFI44L, HERC6, SLC1A2, OLAH, | 0.871 | 0.861 | 0.881 | 0.918 | 0.864 | 0.971 | 0.922 | 0.839 | 1 |
| FAM20A, MMP8 | | | | | | | | | |
| OAS1, IFIT1, IFI44L, SIGLEC1, ISG15, SLC1A2, | 0.871 | 0.861 | 0.880 | 0.913 | 0.854 | 0.972 | 0.937 | 0.863 | 1 |
| FAM20A | | | | | | | | | |
| RSAD2, IFI27, IFI44L, HERC6, SLC1A2, IL1R2, | 0.871 | 0.862 | 0.880 | 0.921 | 0.875 | 0.968 | 0.926 | 0.859 | 0.993 |
| MMP8 | | | | | | | | | |
| IFIT1, IFI44L, SLC1A2, IL1R2, OLAH, FAM20A, | 0.871 | 0.862 | 0.880 | 0.917 | 0.866 | 0.969 | 0.930 | 0.852 | 1 |
| MMP8 | | | | | | | | | |
| OAS1, SIGLEC1, HERC6, SLC1A2, OLAH, | 0.871 | 0.860 | 0.881 | 0.914 | 0.859 | 0.969 | 0.943 | 0.875 | 1 |
| FAM20A, RETN | | | | | | | | | |
| RSAD2, IFI27, IFI44L, ISG15, FAM20A, RETN, | 0.871 | 0.860 | 0.882 | 0.914 | 0.850 | 0.977 | 0.942 | 0.867 | 1 |
| MMP8 | | | | | | | | | |
| RSAD2, OAS1, IFIT1, IFI44L, SIGLEC1, | 0.871 | 0.861 | 0.880 | 0.905 | 0.836 | 0.974 | 0.942 | 0.881 | 1 |
| FAM20A, RETN | | | | | | | | | |
| IFI27, OAS1, ISG15, SLC1A2, IL1R2, RETN, | 0.871 | 0.862 | 0.879 | 0.923 | 0.873 | 0.972 | 0.930 | 0.862 | 0.999 |
| MMP8 | | | | | | | | | |
| OAS1, IFIT1, SIGLEC1, SLC1A2, FAM20A, | 0.871 | 0.861 | 0.881 | 0.915 | 0.857 | 0.973 | 0.952 | 0.884 | 1 |
| RETN, MMP8 | | | | | | | | | |
| IFIT1, IFI44L, SIGLEC1, ISG15, SLC1A2, | 0.871 | 0.861 | 0.880 | 0.912 | 0.854 | 0.971 | 0.936 | 0.863 | 1 |
| FAM20A, RETN | | | | | | | | | |
| IFI27, OAS1, IFI44L, ISG15, SLC1A2, IL1R2, | 0.871 | 0.862 | 0.879 | 0.923 | 0.873 | 0.973 | 0.933 | 0.866 | 0.999 |
| RETN | | | | | | | | | |
| IFIT1, IFI44L, ISG15, SLC1A2, OLAH, FAM20A, | 0.871 | 0.862 | 0.880 | 0.913 | 0.856 | 0.970 | 0.923 | 0.847 | 0.999 |
| MMP8 | | | | | | | | | |
| RSAD2, OAS1, SIGLEC1, ISG15, HERC6, | 0.871 | 0.861 | 0.881 | 0.907 | 0.845 | 0.970 | 0.950 | 0.893 | 1 |
| OLAH, FAM20A | | | | | | | | | |
| IFI27, OAS1, IFI44L, HERC6, SLC1A2, IL1R2, | 0.871 | 0.862 | 0.880 | 0.920 | 0.875 | 0.966 | 0.929 | 0.863 | 0.995 |
| MMP8 | | | | | | | | | |
| IFIT1, IFI44L, ISG15, HERC6, SLC1A2, OLAH, | 0.871 | 0.861 | 0.881 | 0.916 | 0.862 | 0.970 | 0.916 | 0.830 | 1 |
| FAM20A | | | | | | | | | |
| RSAD2, SIGLEC1, HERC6, SLC1A2, OLAH, | 0.871 | 0.860 | 0.881 | 0.917 | 0.862 | 0.972 | 0.940 | 0.869 | 1 |
| FAM20A, RETN | | | | | | | | | |
| IFI27, OAS1, IFI44L, ISG15, HERC6, SLC1A2, | 0.871 | 0.861 | 0.880 | 0.922 | 0.877 | 0.967 | 0.930 | 0.864 | 0.997 |
| IL1R2 | | | | | | | | | |
| RSAD2, IFI44L, SIGLEC1, ISG15, SLC1A2, | 0.871 | 0.861 | 0.880 | 0.912 | 0.853 | 0.971 | 0.945 | 0.881 | 1 |
| OLAH, FAM20A | | | | | | | | | |
| IFI27, IFIT1, ISG15, HERC6, SLC1A2, IL1R2, | 0.870 | 0.862 | 0.879 | 0.922 | 0.876 | 0.967 | 0.936 | 0.876 | 0.996 |
| MMP8 | | | | | | | | | |
| RSAD2, OAS1, IFIT1, HERC6, IL1R2, FAM20A, | 0.870 | 0.861 | 0.880 | 0.907 | 0.846 | 0.968 | 0.939 | 0.861 | 1 |
| MMP8 | | | | | | | | | |
| RSAD2, IFI27, OAS1, IFIT1, HERC6, FAM20A, | 0.870 | 0.860 | 0.880 | 0.912 | 0.854 | 0.970 | 0.927 | 0.846 | 1 |
| MMP8 | | | | | | | | | |
| RSAD2, IFI44L, SIGLEC1, ISG15, HERC6, | 0.870 | 0.860 | 0.881 | 0.909 | 0.847 | 0.971 | 0.948 | 0.890 | 1 |
| OLAH, FAM20A | | | | | | | | | |
| IFI27, OAS1, IFI44L, ISG15, FAM20A, RETN, | 0.870 | 0.859 | 0.882 | 0.912 | 0.849 | 0.975 | 0.948 | 0.874 | 1 |
| MMP8 | | | | | | | | | |
| RSAD2, IFIT1, IFI44L, SIGLEC1, SLC1A2, | 0.870 | 0.861 | 0.880 | 0.912 | 0.853 | 0.970 | 0.937 | 0.863 | 1 |
| FAM20A, MMP8 | | | | | | | | | |
| RSAD2, OAS1, SIGLEC1, SLC1A2, OLAH, | 0.870 | 0.860 | 0.881 | 0.914 | 0.856 | 0.972 | 0.951 | 0.894 | 1 |
| FAM20A, RETN | | | | | | | | | |
| IFIT1, IFI44L, HERC6, IL1R2, OLAH, FAM20A, | 0.870 | 0.861 | 0.880 | 0.909 | 0.850 | 0.968 | 0.937 | 0.860 | 1 |
| MMP8 | | | | | | | | | |
| RSAD2, OAS1, IFIT1, IFI44L, SIGLEC1, | 0.870 | 0.861 | 0.880 | 0.912 | 0.852 | 0.971 | 0.936 | 0.862 | 1 |
| SLC1A2, FAM20A | | | | | | | | | |
| IFI27, OAS1, IFIT1, IFI44L, SIGLEC1, HERC6, | 0.870 | 0.862 | 0.879 | 0.907 | 0.853 | 0.962 | 0.903 | 0.826 | 0.980 |
| MMP8 | | | | | | | | | |
| IFI27, OAS1, IFIT1, IFI44L, ISG15, SLC1A2, | 0.870 | 0.862 | 0.879 | 0.922 | 0.875 | 0.969 | 0.937 | 0.877 | 0.997 |
| IL1R2 | | | | | | | | | |
| IFI44L, SIGLEC1, ISG15, HERC6, OLAH, | 0.870 | 0.860 | 0.880 | 0.903 | 0.837 | 0.970 | 0.954 | 0.893 | 1 |
| FAM20A, RETN | | | | | | | | | |
| IFI27, IFI44L, ISG15, SLC1A2, FAM20A, RETN, | 0.870 | 0.860 | 0.881 | 0.913 | 0.851 | 0.974 | 0.941 | 0.862 | 1 |
| MMP8 | | | | | | | | | |
| OAS1, SIGLEC1, ISG15, HERC6, OLAH, | 0.870 | 0.860 | 0.881 | 0.905 | 0.842 | 0.968 | 0.958 | 0.907 | 1 |
| FAM20A, MMP8 | | | | | | | | | |
| RSAD2, OAS1, IFIT1, SIGLEC1, ISG15, | 0.870 | 0.860 | 0.880 | 0.902 | 0.832 | 0.971 | 0.946 | 0.877 | 1 |
| FAM20A, MMP8 | | | | | | | | | |
| IFI27, OAS1, IFIT1, SIGLEC1, HERC6, SLC1A2, | 0.870 | 0.862 | 0.879 | 0.912 | 0.859 | 0.966 | 0.917 | 0.847 | 0.988 |
| MMP8 | | | | | | | | | |
| RSAD2, IFIT1, IFI44L, SIGLEC1, ISG15, | 0.870 | 0.861 | 0.879 | 0.910 | 0.851 | 0.970 | 0.936 | 0.861 | 1 |
| SLC1A2, FAM20A | | | | | | | | | |
| IFI27, IFI44L, ISG15, HERC6, SLC1A2, IL1R2, | 0.870 | 0.862 | 0.879 | 0.922 | 0.876 | 0.967 | 0.929 | 0.864 | 0.995 |
| MMP8 | | | | | | | | | |
| IFI27, OAS1, IFI44L, SIGLEC1, SLC1A2, RETN, | 0.870 | 0.861 | 0.879 | 0.915 | 0.858 | 0.972 | 0.950 | 0.885 | 1 |
| MMP8 | | | | | | | | | |
| RSAD2, IFI27, IFIT1, IFI44L, SLC1A2, IL1R2, | 0.870 | 0.862 | 0.878 | 0.922 | 0.875 | 0.968 | 0.940 | 0.882 | 0.999 |
| MMP8 | | | | | | | | | |
| RSAD2, OAS1, IFI44L, SIGLEC1, SLC1A2, | 0.870 | 0.860 | 0.880 | 0.915 | 0.857 | 0.973 | 0.946 | 0.886 | 1 |
| OLAH, FAM20A | | | | | | | | | |
| IFIT1, IFI44L, ISG15, HERC6, IL1R2, OLAH, | 0.870 | 0.861 | 0.879 | 0.909 | 0.850 | 0.968 | 0.933 | 0.853 | 1 |
| FAM20A | | | | | | | | | |
| OAS1, SIGLEC1, ISG15, SLC1A2, OLAH, | 0.870 | 0.860 | 0.880 | 0.914 | 0.857 | 0.972 | 0.948 | 0.886 | 1 |
| FAM20A, RETN | | | | | | | | | |
| IFI27, IFIT1, IFI44L, ISG15, SLC1A2, FAM20A, | 0.870 | 0.860 | 0.880 | 0.911 | 0.849 | 0.973 | 0.938 | 0.856 | 1 |
| RETN | | | | | | | | | |
| IFI27, IFIT1, IFI44L, ISG15, HERC6, SLC1A2, | 0.870 | 0.861 | 0.879 | 0.923 | 0.877 | 0.969 | 0.934 | 0.873 | 0.994 |
| IL1R2 | | | | | | | | | |
| RSAD2, IFI27, IFI44L, ISG15, HERC6, SLC1A2, | 0.870 | 0.861 | 0.879 | 0.920 | 0.873 | 0.966 | 0.927 | 0.860 | 0.995 |
| IL1R2 | | | | | | | | | |
| RSAD2, IFIT1, IFI44L, SIGLEC1, ISG15, | 0.870 | 0.861 | 0.879 | 0.900 | 0.830 | 0.970 | 0.943 | 0.884 | 1 |
| FAM20A, RETN | | | | | | | | | |
| OAS1, IFI44L, SIGLEC1, SLC1A2, OLAH, | 0.870 | 0.860 | 0.880 | 0.914 | 0.855 | 0.972 | 0.949 | 0.892 | 1 |
| FAM20A, RETN | | | | | | | | | |
| RSAD2, IFIT1, IFI44L, SIGLEC1, FAM20A, | 0.870 | 0.860 | 0.880 | 0.908 | 0.841 | 0.975 | 0.945 | 0.883 | 1 |
| RETN, MMP8 | | | | | | | | | |
| OAS1, IFIT1, HERC6, IL1R2, OLAH, FAM20A, | 0.870 | 0.860 | 0.880 | 0.905 | 0.845 | 0.966 | 0.942 | 0.868 | 1 |
| MMP8 | | | | | | | | | |
| RSAD2, IFI27, IFIT1, ISG15, HERC6, SLC1A2, | 0.870 | 0.860 | 0.880 | 0.915 | 0.858 | 0.972 | 0.936 | 0.868 | 1 |
| RETN | | | | | | | | | |
| RSAD2, IFI27, OAS1, IFI44L, SLC1A2, IL1R2, | 0.870 | 0.861 | 0.878 | 0.920 | 0.872 | 0.967 | 0.930 | 0.863 | 0.998 |
| MMP8 | | | | | | | | | |
| RSAD2, IFIT1, IFI44L, SIGLEC1, ISG15, | 0.870 | 0.860 | 0.880 | 0.900 | 0.831 | 0.970 | 0.938 | 0.869 | 1 |
| FAM20A, MMP8 | | | | | | | | | |
| IFI44L, SLC1A2, IL1R2, OLAH, FAM20A, | 0.870 | 0.861 | 0.879 | 0.917 | 0.864 | 0.970 | 0.935 | 0.859 | 1 |
| RETN, MMP8 | | | | | | | | | |
| RSAD2, OAS1, ISG15, HERC6, IL1R2, OLAH, | 0.870 | 0.860 | 0.880 | 0.906 | 0.846 | 0.966 | 0.937 | 0.862 | 1 |
| FAM20A | | | | | | | | | |
| RSAD2, OAS1, IFIT1, IL1R2, OLAH, FAM20A, | 0.870 | 0.861 | 0.879 | 0.908 | 0.847 | 0.970 | 0.945 | 0.873 | 1 |
| MMP8 | | | | | | | | | |
| IFI44L, ISG15, HERC6, SLC1A2, IL1R2, OLAH, | 0.870 | 0.860 | 0.880 | 0.914 | 0.860 | 0.968 | 0.929 | 0.846 | 1 |
| FAM20A | | | | | | | | | |
| IFI27, IFIT1, IFI44L, SIGLEC1, ISG15, SLC1A2, | 0.870 | 0.861 | 0.879 | 0.914 | 0.858 | 0.971 | 0.950 | 0.891 | 1 |
| RETN | | | | | | | | | |
| IFIT1, IFI44L, SIGLEC1, HERC6, IL1R2, RETN, | 0.870 | 0.860 | 0.879 | 0.902 | 0.839 | 0.966 | 0.965 | 0.928 | 1 |
| MMP8 | | | | | | | | | |
| RSAD2, OAS1, IFI44L, HERC6, IL1R2, | 0.870 | 0.860 | 0.879 | 0.912 | 0.854 | 0.970 | 0.930 | 0.846 | 1 |
| FAM20A, MMP8 | | | | | | | | | |
| RSAD2, IFI44L, SIGLEC1, SLC1A2, OLAH, | 0.870 | 0.860 | 0.880 | 0.915 | 0.857 | 0.973 | 0.945 | 0.884 | 1 |
| FAM20A, RETN | | | | | | | | | |
| OAS1, IFI44L, SIGLEC1, HERC6, OLAH, | 0.870 | 0.859 | 0.880 | 0.908 | 0.846 | 0.970 | 0.958 | 0.904 | 1 |
| FAM20A, MMP8 | | | | | | | | | |
| RSAD2, SIGLEC1, ISG15, HERC6, OLAH, | 0.870 | 0.860 | 0.880 | 0.908 | 0.847 | 0.970 | 0.949 | 0.892 | 1 |
| FAM20A, RETN | | | | | | | | | |
| SIGLEC1, ISG15, HERC6, SLC1A2, OLAH, | 0.870 | 0.859 | 0.880 | 0.912 | 0.855 | 0.970 | 0.945 | 0.871 | 1 |
| FAM20A, RETN | | | | | | | | | |
| RSAD2, IFI27, IFIT1, SIGLEC1, ISG15, SLC1A2, | 0.870 | 0.861 | 0.878 | 0.915 | 0.859 | 0.971 | 0.953 | 0.896 | 1 |
| RETN | | | | | | | | | |
| OAS1, IFIT1, IFI44L, SIGLEC1, SLC1A2, | 0.870 | 0.860 | 0.879 | 0.914 | 0.855 | 0.972 | 0.936 | 0.863 | 1 |
| FAM20A, RETN | | | | | | | | | |
| RSAD2, IFI27, IFI44L, ISG15, HERC6, SLC1A2, | 0.870 | 0.860 | 0.879 | 0.914 | 0.857 | 0.971 | 0.938 | 0.872 | 1 |
| RETN | | | | | | | | | |
| RSAD2, IFI27, OAS1, ISG15, SLC1A2, IL1R2, | 0.870 | 0.861 | 0.878 | 0.920 | 0.873 | 0.968 | 0.932 | 0.864 | 0.999 |
| MMP8 | | | | | | | | | |
| RSAD2, SIGLEC1, ISG15, SLC1A2, OLAH, | 0.870 | 0.860 | 0.880 | 0.915 | 0.857 | 0.972 | 0.949 | 0.888 | 1 |
| FAM20A, RETN | | | | | | | | | |
| OAS1, IFI44L, SIGLEC1, HERC6, FAM20A, | 0.870 | 0.860 | 0.879 | 0.907 | 0.842 | 0.972 | 0.951 | 0.886 | 1 |
| RETN, MMP8 | | | | | | | | | |
| RSAD2, IFI27, IFIT1, IFI44L, SIGLEC1, SLC1A2, | 0.869 | 0.861 | 0.878 | 0.916 | 0.860 | 0.972 | 0.952 | 0.895 | 1 |
| RETN | | | | | | | | | |
| OAS1, IFI44L, SIGLEC1, ISG15, OLAH, | 0.869 | 0.859 | 0.880 | 0.904 | 0.837 | 0.971 | 0.961 | 0.915 | 1 |
| FAM20A, MMP8 | | | | | | | | | |
| RSAD2, IFI27, IFIT1, IFI44L, ISG15, SLC1A2, | 0.869 | 0.861 | 0.878 | 0.922 | 0.876 | 0.969 | 0.933 | 0.872 | 0.994 |
| IL1R2 | | | | | | | | | |
| RSAD2, IFI27, OAS1, IFIT1, FAM20A, RETN, | 0.869 | 0.859 | 0.880 | 0.912 | 0.850 | 0.973 | 0.943 | 0.867 | 1 |
| MMP8 | | | | | | | | | |
| IFI27, OAS1, IFIT1, IFI44L, ISG15, HERC6, | 0.869 | 0.859 | 0.879 | 0.907 | 0.844 | 0.970 | 0.925 | 0.841 | 1 |
| FAM20A | | | | | | | | | |
| IFI27, OAS1, IFIT1, ISG15, FAM20A, RETN, | 0.869 | 0.859 | 0.880 | 0.911 | 0.848 | 0.973 | 0.945 | 0.869 | 1 |
| MMP8 | | | | | | | | | |
| OAS1, IFIT1, IFI44L, HERC6, SLC1A2, OLAH, | 0.869 | 0.859 | 0.880 | 0.917 | 0.863 | 0.970 | 0.913 | 0.826 | 1 |
| FAM20A | | | | | | | | | |
| RSAD2, IFIT1, SIGLEC1, ISG15, FAM20A, | 0.869 | 0.860 | 0.879 | 0.904 | 0.837 | 0.972 | 0.948 | 0.886 | 1 |
| RETN, MMP8 | | | | | | | | | |
| IFI27, IFIT1, IFI44L, HERC6, SLC1A2, FAM20A, | 0.869 | 0.859 | 0.879 | 0.913 | 0.857 | 0.970 | 0.914 | 0.825 | 1 |
| MMP8 | | | | | | | | | |
| IFI44L, ISG15, HERC6, IL1R2, OLAH, FAM20A, | 0.869 | 0.860 | 0.879 | 0.907 | 0.846 | 0.968 | 0.940 | 0.868 | 1 |
| RETN | | | | | | | | | |
| RSAD2, OAS1, IFI44L, SIGLEC1, HERC6, | 0.869 | 0.859 | 0.879 | 0.906 | 0.844 | 0.969 | 0.947 | 0.889 | 1 |
| OLAH, FAM20A | | | | | | | | | |
| RSAD2, IFI44L, SIGLEC1, ISG15, OLAH, | 0.869 | 0.859 | 0.879 | 0.904 | 0.837 | 0.970 | 0.962 | 0.920 | 1 |
| FAM20A, MMP8 | | | | | | | | | |
| RSAD2, IFI27, IFI44L, ISG15, SLC1A2, IL1R2, | 0.869 | 0.861 | 0.877 | 0.920 | 0.873 | 0.968 | 0.928 | 0.861 | 0.996 |
| MMP8 | | | | | | | | | |
| RSAD2, IFI27, OAS1, IFIT1, SIGLEC1, HERC6, | 0.869 | 0.860 | 0.878 | 0.908 | 0.851 | 0.964 | 0.904 | 0.826 | 0.983 |
| MMP8 | | | | | | | | | |
| IFIT1, SIGLEC1, ISG15, HERC6, IL1R2, RETN, | 0.869 | 0.860 | 0.879 | 0.904 | 0.841 | 0.967 | 0.964 | 0.927 | 1 |
| MMP8 | | | | | | | | | |
| OAS1, SIGLEC1, ISG15, OLAH, FAM20A, | 0.869 | 0.859 | 0.880 | 0.905 | 0.838 | 0.972 | 0.967 | 0.928 | 1 |
| RETN, MMP8 | | | | | | | | | |
| RSAD2, IFI44L, ISG15, HERC6, IL1R2, OLAH, | 0.869 | 0.859 | 0.879 | 0.909 | 0.850 | 0.968 | 0.936 | 0.863 | 1 |
| FAM20A | | | | | | | | | |
| RSAD2, ISG15, HERC6, IL1R2, OLAH, | 0.869 | 0.859 | 0.879 | 0.908 | 0.850 | 0.967 | 0.936 | 0.857 | 1 |
| FAM20A, MMP8 | | | | | | | | | |
| RSAD2, OAS1, ISG15, HERC6, IL1R2, FAM20A, | 0.869 | 0.859 | 0.879 | 0.908 | 0.849 | 0.967 | 0.932 | 0.849 | 1 |
| MMP8 | | | | | | | | | |
| RSAD2, OAS1, SIGLEC1, HERC6, OLAH, | 0.869 | 0.859 | 0.879 | 0.906 | 0.843 | 0.969 | 0.951 | 0.895 | 1 |
| FAM20A, RETN | | | | | | | | | |
| IFI44L, SIGLEC1, HERC6, SLC1A2, OLAH, | 0.869 | 0.859 | 0.880 | 0.914 | 0.857 | 0.972 | 0.947 | 0.874 | 1 |
| FAM20A, RETN | | | | | | | | | |
| RSAD2, OAS1, SIGLEC1, HERC6, IL1R2, RETN, | 0.869 | 0.859 | 0.879 | 0.902 | 0.838 | 0.966 | 0.966 | 0.931 | 1 |
| MMP8 | | | | | | | | | |
| OAS1, IFIT1, SIGLEC1, ISG15, SLC1A2, | 0.869 | 0.859 | 0.879 | 0.910 | 0.849 | 0.970 | 0.942 | 0.868 | 1 |
| FAM20A, RETN | | | | | | | | | |
| RSAD2, IFI27, OAS1, IFI44L, ISG15, SLC1A2, | 0.869 | 0.861 | 0.877 | 0.922 | 0.876 | 0.968 | 0.929 | 0.861 | 0.998 |
| IL1R2 | | | | | | | | | |
| RSAD2, OAS1, IFIT1, SIGLEC1, ISG15, | 0.869 | 0.859 | 0.879 | 0.907 | 0.845 | 0.968 | 0.943 | 0.871 | 1 |
| SLC1A2, FAM20A | | | | | | | | | |
| IFIT1, ISG15, HERC6, IL1R2, OLAH, FAM20A, | 0.869 | 0.860 | 0.879 | 0.907 | 0.848 | 0.966 | 0.945 | 0.872 | 1 |
| MMP8 | | | | | | | | | |
| OAS1, IFI44L, SIGLEC1, ISG15, HERC6, OLAH, | 0.869 | 0.859 | 0.879 | 0.905 | 0.841 | 0.969 | 0.954 | 0.899 | 1 |
| FAM20A | | | | | | | | | |
| OAS1, SIGLEC1, ISG15, HERC6, SLC1A2, | 0.869 | 0.859 | 0.880 | 0.911 | 0.853 | 0.968 | 0.946 | 0.876 | 1 |
| FAM20A, RETN | | | | | | | | | |
| OAS1, IFI44L, SIGLEC1, HERC6, IL1R2, RETN, | 0.869 | 0.860 | 0.879 | 0.903 | 0.840 | 0.966 | 0.962 | 0.923 | 1 |
| MMP8 | | | | | | | | | |
| RSAD2, OAS1, SIGLEC1, ISG15, HERC6, | 0.869 | 0.859 | 0.879 | 0.910 | 0.854 | 0.967 | 0.939 | 0.863 | 1 |
| SLC1A2, FAM20A | | | | | | | | | |
| IFIT1, IFI44L, ISG15, HERC6, IL1R2, FAM20A, | 0.869 | 0.860 | 0.878 | 0.911 | 0.852 | 0.970 | 0.934 | 0.855 | 1 |
| RETN | | | | | | | | | |
| OAS1, IFIT1, SIGLEC1, ISG15, HERC6, IL1R2, | 0.869 | 0.860 | 0.879 | 0.906 | 0.847 | 0.966 | 0.954 | 0.910 | 0.999 |
| RETN | | | | | | | | | |
| OAS1, IFIT1, ISG15, HERC6, IL1R2, FAM20A, | 0.869 | 0.859 | 0.879 | 0.907 | 0.847 | 0.967 | 0.935 | 0.853 | 1 |
| RETN | | | | | | | | | |
| RSAD2, SIGLEC1, ISG15, HERC6, SLC1A2, | 0.869 | 0.859 | 0.879 | 0.912 | 0.855 | 0.970 | 0.942 | 0.869 | 1 |
| FAM20A, RETN | | | | | | | | | |
| RSAD2, IFI44L, SLC1A2, IL1R2, OLAH, | 0.869 | 0.860 | 0.879 | 0.918 | 0.867 | 0.969 | 0.927 | 0.846 | 1 |
| FAM20A, MMP8 | | | | | | | | | |
| IFIT1, IFI44L, SIGLEC1, ISG15, SLC1A2, OLAH, | 0.869 | 0.860 | 0.878 | 0.912 | 0.857 | 0.968 | 0.950 | 0.903 | 0.997 |
| MMP8 | | | | | | | | | |
| OAS1, ISG15, HERC6, IL1R2, FAM20A, RETN, | 0.869 | 0.859 | 0.879 | 0.910 | 0.851 | 0.969 | 0.934 | 0.852 | 1 |
| MMP8 | | | | | | | | | |
| IFIT1, SIGLEC1, HERC6, SLC1A2, OLAH, | 0.869 | 0.859 | 0.879 | 0.914 | 0.859 | 0.969 | 0.951 | 0.906 | 0.997 |
| RETN, MMP8 | | | | | | | | | |
| OAS1, ISG15, HERC6, IL1R2, OLAH, FAM20A, | 0.869 | 0.859 | 0.879 | 0.909 | 0.851 | 0.968 | 0.938 | 0.860 | 1 |
| MMP8 | | | | | | | | | |
| RSAD2, IFI27, IFIT1, IFI44L, SIGLEC1, RETN, | 0.869 | 0.859 | 0.879 | 0.910 | 0.849 | 0.971 | 0.951 | 0.893 | 1 |
| MMP8 | | | | | | | | | |
| RSAD2, OAS1, IFIT1, SIGLEC1, HERC6, IL1R2, | 0.869 | 0.859 | 0.878 | 0.905 | 0.842 | 0.967 | 0.952 | 0.906 | 0.998 |
| RETN | | | | | | | | | |
| OAS1, IFIT1, IFI44L, SIGLEC1, HERC6, IL1R2, | 0.869 | 0.860 | 0.878 | 0.905 | 0.842 | 0.967 | 0.951 | 0.905 | 0.997 |
| RETN | | | | | | | | | |
| OAS1, IFIT1, SIGLEC1, ISG15, SLC1A2, | 0.869 | 0.859 | 0.878 | 0.909 | 0.848 | 0.969 | 0.946 | 0.872 | 1 |
| FAM20A, MMP8 | | | | | | | | | |
| IFI27, OAS1, IFIT1, IFI44L, SLC1A2, FAM20A, | 0.869 | 0.859 | 0.879 | 0.911 | 0.849 | 0.973 | 0.936 | 0.853 | 1 |
| RETN | | | | | | | | | |
| RSAD2, SIGLEC1, ISG15, OLAH, FAM20A, | 0.869 | 0.859 | 0.879 | 0.908 | 0.844 | 0.972 | 0.963 | 0.920 | 1 |
| RETN, MMP8 | | | | | | | | | |
| RSAD2, OAS1, IFIT1, ISG15, HERC6, IL1R2, | 0.869 | 0.859 | 0.878 | 0.906 | 0.845 | 0.966 | 0.935 | 0.855 | 1 |
| FAM20A | | | | | | | | | |
| IFIT1, IFI44L, HERC6, SLC1A2, IL1R2, OLAH, | 0.869 | 0.859 | 0.879 | 0.918 | 0.866 | 0.969 | 0.920 | 0.832 | 1 |
| FAM20A | | | | | | | | | |
| RSAD2, OAS1, IFI44L, ISG15, HERC6, IL1R2, | 0.869 | 0.859 | 0.878 | 0.909 | 0.850 | 0.968 | 0.936 | 0.860 | 1 |
| FAM20A | | | | | | | | | |
| RSAD2, IFI27, IFIT1, SIGLEC1, ISG15, RETN, | 0.869 | 0.859 | 0.879 | 0.910 | 0.849 | 0.971 | 0.955 | 0.900 | 1 |
| MMP8 | | | | | | | | | |
| OAS1, SIGLEC1, ISG15, HERC6, IL1R2, RETN, | 0.869 | 0.859 | 0.878 | 0.906 | 0.845 | 0.967 | 0.972 | 0.940 | 1 |
| MMP8 | | | | | | | | | |
| OAS1, SIGLEC1, ISG15, HERC6, OLAH, | 0.869 | 0.859 | 0.879 | 0.907 | 0.845 | 0.970 | 0.955 | 0.902 | 1 |
| FAM20A, RETN | | | | | | | | | |
| OAS1, IFIT1, ISG15, HERC6, IL1R2, FAM20A, | 0.869 | 0.859 | 0.878 | 0.907 | 0.846 | 0.967 | 0.937 | 0.859 | 1 |
| MMP8 | | | | | | | | | |
| RSAD2, IFI27, OAS1, SIGLEC1, HERC6, | 0.869 | 0.859 | 0.878 | 0.913 | 0.859 | 0.966 | 0.929 | 0.862 | 0.997 |
| SLC1A2, MMP8 | | | | | | | | | |
| IFIT1, IFI44L, ISG15, HERC6, IL1R2, FAM20A, | 0.869 | 0.859 | 0.878 | 0.910 | 0.850 | 0.969 | 0.932 | 0.847 | 1 |
| MMP8 | | | | | | | | | |
| RSAD2, IFI44L, HERC6, SLC1A2, OLAH, | 0.869 | 0.858 | 0.879 | 0.916 | 0.862 | 0.970 | 0.922 | 0.843 | 1 |
| FAM20A, MMP8 | | | | | | | | | |
| IFIT1, IFI44L, SIGLEC1, HERC6, SLC1A2, | 0.869 | 0.859 | 0.879 | 0.913 | 0.858 | 0.968 | 0.941 | 0.889 | 0.993 |
| OLAH, MMP8 | | | | | | | | | |
| IFI27, OAS1, IFI44L, ISG15, SLC1A2, FAM20A, | 0.869 | 0.858 | 0.879 | 0.911 | 0.848 | 0.973 | 0.938 | 0.856 | 1 |
| RETN | | | | | | | | | |
| OAS1, IFI44L, SIGLEC1, OLAH, FAM20A, | 0.869 | 0.858 | 0.879 | 0.906 | 0.840 | 0.972 | 0.967 | 0.927 | 1 |
| RETN, MMP8 | | | | | | | | | |
| IFIT1, SIGLEC1, HERC6, IL1R2, OLAH, RETN, | 0.869 | 0.859 | 0.878 | 0.904 | 0.842 | 0.967 | 0.964 | 0.927 | 1 |
| MMP8 | | | | | | | | | |
| ISG15, HERC6, IL1R2, OLAH, FAM20A, RETN, | 0.869 | 0.859 | 0.879 | 0.909 | 0.850 | 0.969 | 0.939 | 0.863 | 1 |
| MMP8 | | | | | | | | | |
| IFI27, IFIT1, IFI44L, SIGLEC1, ISG15, HERC6, | 0.869 | 0.860 | 0.877 | 0.906 | 0.849 | 0.963 | 0.908 | 0.833 | 0.982 |
| MMP8 | | | | | | | | | |
| IFI44L, ISG15, SLC1A2, IL1R2, OLAH, | 0.869 | 0.859 | 0.878 | 0.913 | 0.858 | 0.968 | 0.927 | 0.849 | 1 |
| FAM20A, RETN | | | | | | | | | |
| RSAD2, IFI44L, SIGLEC1, HERC6, FAM20A, | 0.869 | 0.859 | 0.878 | 0.909 | 0.846 | 0.972 | 0.949 | 0.882 | 1 |
| RETN, MMP8 | | | | | | | | | |
| IFI27, IFIT1, SIGLEC1, ISG15, SLC1A2, RETN, | 0.869 | 0.859 | 0.878 | 0.915 | 0.860 | 0.971 | 0.953 | 0.896 | 1 |
| MMP8 | | | | | | | | | |
| RSAD2, IFI27, IFIT1, IFI44L, HERC6, SLC1A2, | 0.869 | 0.859 | 0.878 | 0.913 | 0.854 | 0.971 | 0.937 | 0.869 | 1 |
| RETN | | | | | | | | | |
| IFI44L, ISG15, HERC6, IL1R2, FAM20A, RETN, | 0.869 | 0.859 | 0.878 | 0.910 | 0.849 | 0.970 | 0.941 | 0.864 | 1 |
| MMP8 | | | | | | | | | |
| RSAD2, IFI27, ISG15, HERC6, SLC1A2, RETN, | 0.869 | 0.859 | 0.878 | 0.915 | 0.858 | 0.971 | 0.936 | 0.868 | 1 |
| MMP8 | | | | | | | | | |
| OAS1, IFI44L, HERC6, IL1R2, OLAH, FAM20A, | 0.869 | 0.859 | 0.878 | 0.909 | 0.849 | 0.968 | 0.940 | 0.865 | 1 |
| MMP8 | | | | | | | | | |
| IFIT1, IFI44L, HERC6, SLC1A2, OLAH, | 0.868 | 0.858 | 0.879 | 0.917 | 0.863 | 0.970 | 0.917 | 0.832 | 1 |
| FAM20A, RETN | | | | | | | | | |
| RSAD2, ISG15, HERC6, IL1R2, FAM20A, RETN, | 0.868 | 0.859 | 0.878 | 0.910 | 0.850 | 0.969 | 0.932 | 0.848 | 1 |
| MMP8 | | | | | | | | | |
| RSAD2, IFI44L, SIGLEC1, ISG15, HERC6, | 0.868 | 0.859 | 0.878 | 0.910 | 0.849 | 0.971 | 0.947 | 0.881 | 1 |
| FAM20A, RETN | | | | | | | | | |
| RSAD2, OAS1, IFI44L, HERC6, IL1R2, OLAH, | 0.868 | 0.858 | 0.878 | 0.907 | 0.846 | 0.968 | 0.935 | 0.860 | 1 |
| FAM20A | | | | | | | | | |
| IFIT1, SIGLEC1, HERC6, SLC1A2, IL1R2, | 0.868 | 0.859 | 0.878 | 0.912 | 0.859 | 0.966 | 0.962 | 0.924 | 1 |
| OLAH, MMP8 | | | | | | | | | |
| RSAD2, IFI27, OAS1, ISG15, FAM20A, RETN, | 0.868 | 0.858 | 0.879 | 0.910 | 0.848 | 0.971 | 0.942 | 0.865 | 1 |
| MMP8 | | | | | | | | | |
| RSAD2, SIGLEC1, HERC6, SLC1A2, IL1R2, | 0.868 | 0.859 | 0.878 | 0.912 | 0.859 | 0.966 | 0.963 | 0.925 | 1 |
| OLAH, MMP8 | | | | | | | | | |
| IFIT1, SIGLEC1, ISG15, SLC1A2, FAM20A, | 0.868 | 0.859 | 0.878 | 0.914 | 0.856 | 0.971 | 0.948 | 0.878 | 1 |
| RETN, MMP8 | | | | | | | | | |
| IFI44L, SIGLEC1, ISG15, SLC1A2, IL1R2, | 0.868 | 0.860 | 0.877 | 0.913 | 0.860 | 0.966 | 0.965 | 0.929 | 1 |
| OLAH, MMP8 | | | | | | | | | |
| RSAD2, IFI27, OAS1, IFI44L, SIGLEC1, HERC6, | 0.868 | 0.860 | 0.877 | 0.906 | 0.849 | 0.963 | 0.913 | 0.839 | 0.987 |
| MMP8 | | | | | | | | | |
| OAS1, IFI44L, SIGLEC1, ISG15, HERC6, | 0.868 | 0.859 | 0.878 | 0.907 | 0.845 | 0.969 | 0.949 | 0.885 | 1 |
| FAM20A, RETN | | | | | | | | | |
| OAS1, IFIT1, SIGLEC1, HERC6, OLAH, RETN, | 0.868 | 0.858 | 0.878 | 0.901 | 0.836 | 0.967 | 0.953 | 0.909 | 0.997 |
| MMP8 | | | | | | | | | |
| RSAD2, OAS1, IFI44L, IL1R2, OLAH, FAM20A, | 0.868 | 0.858 | 0.878 | 0.907 | 0.846 | 0.969 | 0.942 | 0.873 | 1 |
| MMP8 | | | | | | | | | |
| IFI44L, HERC6, IL1R2, OLAH, FAM20A, RETN, | 0.868 | 0.858 | 0.878 | 0.912 | 0.852 | 0.973 | 0.946 | 0.874 | 1 |
| MMP8 | | | | | | | | | |
| IFIT1, ISG15, HERC6, IL1R2, FAM20A, RETN, | 0.868 | 0.859 | 0.878 | 0.910 | 0.850 | 0.970 | 0.938 | 0.859 | 1 |
| MMP8 | | | | | | | | | |
| OAS1, IFI44L, ISG15, HERC6, IL1R2, OLAH, | 0.868 | 0.858 | 0.878 | 0.906 | 0.845 | 0.966 | 0.938 | 0.864 | 1 |
| FAM20A | | | | | | | | | |
| RSAD2, OAS1, ISG15, HERC6, IL1R2, FAM20A, | 0.868 | 0.858 | 0.878 | 0.907 | 0.847 | 0.967 | 0.933 | 0.849 | 1 |
| RETN | | | | | | | | | |
| OAS1, SIGLEC1, HERC6, SLC1A2, IL1R2, | 0.868 | 0.859 | 0.878 | 0.912 | 0.859 | 0.966 | 0.961 | 0.922 | 1 |
| OLAH, MMP8 | | | | | | | | | |
| RSAD2, SIGLEC1, ISG15, HERC6, SLC1A2, | 0.868 | 0.858 | 0.878 | 0.908 | 0.850 | 0.966 | 0.942 | 0.867 | 1 |
| FAM20A, MMP8 | | | | | | | | | |
| IFI44L, ISG15, HERC6, SLC1A2, OLAH, | 0.868 | 0.858 | 0.878 | 0.914 | 0.858 | 0.969 | 0.927 | 0.850 | 1 |
| FAM20A, MMP8 | | | | | | | | | |
| RSAD2, IFI27, IFI44L, SIGLEC1, ISG15, RETN, | 0.868 | 0.858 | 0.878 | 0.911 | 0.850 | 0.971 | 0.952 | 0.895 | 1 |
| MMP8 | | | | | | | | | |
| RSAD2, IFIT1, IFI44L, HERC6, SLC1A2, OLAH, | 0.868 | 0.858 | 0.878 | 0.916 | 0.863 | 0.970 | 0.914 | 0.828 | 1 |
| FAM20A | | | | | | | | | |
| IFI27, IFIT1, IFI44L, SLC1A2, FAM20A, RETN, | 0.868 | 0.858 | 0.879 | 0.914 | 0.852 | 0.975 | 0.940 | 0.861 | 1 |
| MMP8 | | | | | | | | | |
| IFIT1, SIGLEC1, ISG15, HERC6, SLC1A2, | 0.868 | 0.858 | 0.878 | 0.913 | 0.858 | 0.968 | 0.947 | 0.898 | 0.996 |
| OLAH, MMP8 | | | | | | | | | |
| OAS1, IFIT1, IFI44L, SIGLEC1, HERC6, IL1R2, | 0.868 | 0.859 | 0.877 | 0.902 | 0.843 | 0.961 | 0.960 | 0.919 | 1 |
| MMP8 | | | | | | | | | |
| OAS1, IFIT1, SIGLEC1, HERC6, SLC1A2, | 0.868 | 0.858 | 0.878 | 0.913 | 0.858 | 0.968 | 0.947 | 0.898 | 0.995 |
| OLAH, MMP8 | | | | | | | | | |
| IFI27, OAS1, SIGLEC1, ISG15, HERC6, SLC1A2, | 0.868 | 0.859 | 0.877 | 0.911 | 0.857 | 0.964 | 0.923 | 0.853 | 0.992 |
| MMP8 | | | | | | | | | |
| OAS1, IFIT1, HERC6, IL1R2, FAM20A, RETN, | 0.868 | 0.858 | 0.878 | 0.908 | 0.845 | 0.970 | 0.941 | 0.862 | 1 |
| MMP8 | | | | | | | | | |
| RSAD2, OAS1, IFIT1, SIGLEC1, SLC1A2, | 0.868 | 0.858 | 0.878 | 0.908 | 0.847 | 0.969 | 0.946 | 0.872 | 1 |
| FAM20A, MMP8 | | | | | | | | | |
| OAS1, IFIT1, ISG15, HERC6, IL1R2, OLAH, | 0.868 | 0.858 | 0.878 | 0.906 | 0.84 | 0.966 | 0.936 | 0.860 | 1 |
| FAM20A | | | | | | | | | |
| IFIT1, SIGLEC1, ISG15, HERC6, OLAH, RETN, | 0.868 | 0.858 | 0.878 | 0.904 | 0.841 | 0.968 | 0.960 | 0.920 | 1.000 |
| MMP8 | | | | | | | | | |
| RSAD2, ISG15, HERC6, IL1R2, OLAH, | 0.868 | 0.858 | 0.878 | 0.906 | 0.846 | 0.966 | 0.937 | 0.862 | 1 |
| FAM20A, RETN | | | | | | | | | |
| IFIT1, IFI44L, SIGLEC1, ISG15, HERC6, IL1R2, | 0.868 | 0.859 | 0.877 | 0.904 | 0.843 | 0.966 | 0.955 | 0.912 | 0.999 |
| RETN | | | | | | | | | |
| OAS1, IFIT1, IFI44L, HERC6, IL1R2, OLAH, | 0.868 | 0.859 | 0.877 | 0.906 | 0.845 | 0.967 | 0.934 | 0.856 | 1 |
| FAM20A | | | | | | | | | |
| OAS1, SIGLEC1, HERC6, SLC1A2, OLAH, | 0.868 | 0.858 | 0.878 | 0.913 | 0.856 | 0.970 | 0.962 | 0.922 | 1 |
| RETN, MMP8 | | | | | | | | | |
| RSAD2, IFI27, IFI44L, SIGLEC1, ISG15, | 0.868 | 0.859 | 0.877 | 0.914 | 0.858 | 0.971 | 0.950 | 0.890 | 1 |
| SLC1A2, RETN | | | | | | | | | |
| RSAD2, IFIT1, SIGLEC1, SLC1A2, FAM20A, | 0.868 | 0.858 | 0.878 | 0.913 | 0.855 | 0.971 | 0.950 | 0.881 | 1 |
| RETN, MMP8 | | | | | | | | | |
| IFIT1, ISG15, HERC6, IL1R2, OLAH, FAM20A, | 0.868 | 0.858 | 0.878 | 0.906 | 0.846 | 0.967 | 0.936 | 0.860 | 1 |
| RETN | | | | | | | | | |
| IFIT1, HERC6, SLC1A2, IL1R2, OLAH, | 0.868 | 0.858 | 0.878 | 0.916 | 0.865 | 0.968 | 0.932 | 0.850 | 1 |
| FAM20A, MMP8 | | | | | | | | | |
| RSAD2, IFI27, IFIT1, IFI44L, SLC1A2, FAM20A, | 0.868 | 0.858 | 0.878 | 0.910 | 0.847 | 0.972 | 0.938 | 0.857 | 1 |
| RETN | | | | | | | | | |
| RSAD2, IFI44L, HERC6, IL1R2, OLAH, | 0.868 | 0.858 | 0.878 | 0.910 | 0.851 | 0.968 | 0.933 | 0.854 | 1 |
| FAM20A, MMP8 | | | | | | | | | |
| RSAD2, IFIT1, IFI44L, SLC1A2, OLAH, | 0.868 | 0.858 | 0.877 | 0.913 | 0.856 | 0.970 | 0.924 | 0.849 | 0.998 |
| FAM20A, MMP8 | | | | | | | | | |
| IFI44L, ISG15, SLC1A2, OLAH, FAM20A, | 0.868 | 0.858 | 0.878 | 0.915 | 0.858 | 0.971 | 0.928 | 0.855 | 1 |
| RETN, MMP8 | | | | | | | | | |
| OAS1, HERC6, IL1R2, OLAH, FAM20A, RETN, | 0.868 | 0.858 | 0.878 | 0.908 | 0.848 | 0.969 | 0.942 | 0.868 | 1 |
| MMP8 | | | | | | | | | |
| IFI44L, SIGLEC1, HERC6, SLC1A2, FAM20A, | 0.868 | 0.858 | 0.878 | 0.906 | 0.843 | 0.969 | 0.954 | 0.881 | 1 |
| RETN, MMP8 | | | | | | | | | |
| OAS1, IFIT1, IFI44L, HERC6, IL1R2, FAM20A, | 0.868 | 0.859 | 0.877 | 0.909 | 0.849 | 0.969 | 0.934 | 0.851 | 1 |
| MMP8 | | | | | | | | | |
| RSAD2, IFIT1, IFI44L, SIGLEC1, SLC1A2, | 0.868 | 0.858 | 0.877 | 0.913 | 0.854 | 0.972 | 0.936 | 0.863 | 1 |
| FAM20A, RETN | | | | | | | | | |
| RSAD2, IFI27, IFIT1, ISG15, FAM20A, RETN, | 0.868 | 0.857 | 0.878 | 0.911 | 0.849 | 0.974 | 0.943 | 0.867 | 1 |
| MMP8 | | | | | | | | | |
| OAS1, IFIT1, IFI44L, ISG15, HERC6, IL1R2, | 0.868 | 0.858 | 0.877 | 0.908 | 0.850 | 0.967 | 0.934 | 0.854 | 1 |
| FAM20A | | | | | | | | | |
| RSAD2, OAS1, IFIT1, HERC6, IL1R2, OLAH, | 0.868 | 0.858 | 0.878 | 0.905 | 0.844 | 0.967 | 0.938 | 0.864 | 1 |
| FAM20A | | | | | | | | | |
| IFI27, IFIT1, IFI44L, HERC6, SLC1A2, RETN, | 0.868 | 0.858 | 0.877 | 0.911 | 0.851 | 0.971 | 0.927 | 0.853 | 1 |
| MMP8 | | | | | | | | | |
| RSAD2, IFIT1, ISG15, HERC6, IL1R2, FAM20A, | 0.868 | 0.858 | 0.877 | 0.908 | 0.849 | 0.967 | 0.936 | 0.858 | 1 |
| MMP8 | | | | | | | | | |
| IFIT1, IFI44L, ISG15, HERC6, OLAH, FAM20A, | 0.868 | 0.858 | 0.877 | 0.905 | 0.843 | 0.968 | 0.937 | 0.868 | 1 |
| RETN | | | | | | | | | |
| RSAD2, IFIT1, ISG15, HERC6, IL1R2, FAM20A, | 0.868 | 0.858 | 0.877 | 0.908 | 0.848 | 0.967 | 0.935 | 0.853 | 1 |
| RETN | | | | | | | | | |
| RSAD2, IFIT1, SIGLEC1, HERC6, IL1R2, RETN, | 0.868 | 0.858 | 0.877 | 0.904 | 0.841 | 0.966 | 0.963 | 0.925 | 1 |
| MMP8 | | | | | | | | | |
| IFIT1, HERC6, IL1R2, OLAH, FAM20A, RETN, | 0.868 | 0.858 | 0.878 | 0.908 | 0.846 | 0.969 | 0.942 | 0.868 | 1 |
| MMP8 | | | | | | | | | |
| RSAD2, OAS1, SIGLEC1, ISG15, HERC6, | 0.868 | 0.858 | 0.878 | 0.902 | 0.838 | 0.967 | 0.940 | 0.869 | 1 |
| FAM20A, MMP8 | | | | | | | | | |
| RSAD2, HERC6, SLC1A2, IL1R2, OLAH, | 0.868 | 0.858 | 0.877 | 0.914 | 0.861 | 0.966 | 0.926 | 0.837 | 1 |
| FAM20A, MMP8 | | | | | | | | | |
| RSAD2, OAS1, IFIT1, SIGLEC1, SLC1A2, | 0.868 | 0.858 | 0.878 | 0.909 | 0.849 | 0.970 | 0.945 | 0.873 | 1 |
| FAM20A, RETN | | | | | | | | | |
| IFIT1, SIGLEC1, ISG15, SLC1A2, IL1R2, OLAH, | 0.868 | 0.859 | 0.877 | 0.913 | 0.859 | 0.966 | 0.966 | 0.931 | 1 |
| MMP8 | | | | | | | | | |
| IFI27, IFIT1, ISG15, HERC6, SLC1A2, RETN, | 0.868 | 0.858 | 0.878 | 0.912 | 0.854 | 0.971 | 0.928 | 0.855 | 1 |
| MMP8 | | | | | | | | | |
| IFIT1, IFI44L, SIGLEC1, HERC6, OLAH, RETN, | 0.868 | 0.858 | 0.878 | 0.901 | 0.835 | 0.966 | 0.958 | 0.917 | 0.999 |
| MMP8 | | | | | | | | | |
| OAS1, IFI44L, SIGLEC1, HERC6, OLAH, | 0.868 | 0.858 | 0.877 | 0.906 | 0.843 | 0.970 | 0.954 | 0.899 | 1 |
| FAM20A, RETN | | | | | | | | | |
| RSAD2, IFIT1, IFI44L, ISG15, HERC6, IL1R2, | 0.868 | 0.858 | 0.877 | 0.909 | 0.851 | 0.967 | 0.930 | 0.849 | 1 |
| FAM20A | | | | | | | | | |
| RSAD2, OAS1, HERC6, IL1R2, FAM20A, RETN, | 0.868 | 0.858 | 0.877 | 0.906 | 0.844 | 0.968 | 0.938 | 0.857 | 1 |
| MMP8 | | | | | | | | | |
| RSAD2, HERC6, IL1R2, OLAH, FAM20A, | 0.868 | 0.858 | 0.878 | 0.906 | 0.845 | 0.967 | 0.941 | 0.866 | 1 |
| RETN, MMP8 | | | | | | | | | |
| OAS1, HERC6, SLC1A2, IL1R2, OLAH, | 0.867 | 0.858 | 0.877 | 0.914 | 0.862 | 0.966 | 0.925 | 0.838 | 1 |
| FAM20A, MMP8 | | | | | | | | | |
| RSAD2, IFIT1, SIGLEC1, ISG15, SLC1A2, | 0.867 | 0.858 | 0.877 | 0.906 | 0.845 | 0.967 | 0.946 | 0.874 | 1 |
| FAM20A, MMP8 | | | | | | | | | |
| RSAD2, OAS1, HERC6, SLC1A2, IL1R2, OLAH, | 0.867 | 0.857 | 0.877 | 0.913 | 0.858 | 0.967 | 0.926 | 0.840 | 1 |
| FAM20A | | | | | | | | | |
| RSAD2, OAS1, SIGLEC1, HERC6, SLC1A2, | 0.867 | 0.857 | 0.877 | 0.908 | 0.851 | 0.965 | 0.941 | 0.866 | 1 |
| FAM20A, MMP8 | | | | | | | | | |
| RSAD2, IFI27, IFIT1, SIGLEC1, SLC1A2, RETN, | 0.867 | 0.858 | 0.877 | 0.915 | 0.859 | 0.971 | 0.953 | 0.896 | 1 |
| MMP8 | | | | | | | | | |
| RSAD2, OAS1, IFIT1, SIGLEC1, HERC6, | 0.867 | 0.858 | 0.877 | 0.912 | 0.857 | 0.967 | 0.951 | 0.905 | 0.998 |
| SLC1A2, OLAH | | | | | | | | | |
| RSAD2, IFI44L, HERC6, SLC1A2, IL1R2, OLAH, | 0.867 | 0.857 | 0.878 | 0.915 | 0.861 | 0.968 | 0.924 | 0.840 | 1 |
| FAM20A | | | | | | | | | |
| OAS1, ISG15, HERC6, IL1R2, OLAH, FAM20A, | 0.867 | 0.857 | 0.878 | 0.906 | 0.846 | 0.967 | 0.937 | 0.862 | 1 |
| RETN | | | | | | | | | |
| OAS1, ISG15, HERC6, SLC1A2, IL1R2, OLAH, | 0.867 | 0.857 | 0.878 | 0.912 | 0.857 | 0.966 | 0.927 | 0.842 | 1 |
| FAM20A | | | | | | | | | |
| OAS1, IFIT1, IFI44L, SIGLEC1, HERC6, | 0.867 | 0.858 | 0.877 | 0.912 | 0.858 | 0.967 | 0.939 | 0.886 | 0.993 |
| SLC1A2, OLAH | | | | | | | | | |
| IFI27, OAS1, IFI44L, SIGLEC1, ISG15, HERC6, | 0.867 | 0.859 | 0.876 | 0.908 | 0.854 | 0.963 | 0.911 | 0.836 | 0.986 |
| MMP8 | | | | | | | | | |
| RSAD2, IFIT1, HERC6, IL1R2, OLAH, FAM20A, | 0.867 | 0.858 | 0.877 | 0.905 | 0.846 | 0.965 | 0.946 | 0.875 | 1 |
| MMP8 | | | | | | | | | |
| IFI44L, SIGLEC1, ISG15, HERC6, IL1R2, RETN, | 0.867 | 0.858 | 0.877 | 0.901 | 0.837 | 0.965 | 0.964 | 0.927 | 1 |
| MMP8 | | | | | | | | | |
| RSAD2, SIGLEC1, ISG15, HERC6, IL1R2, | 0.867 | 0.858 | 0.877 | 0.906 | 0.844 | 0.968 | 0.963 | 0.925 | 1 |
| RETN, MMP8 | | | | | | | | | |
| RSAD2, IFIT1, ISG15, HERC6, IL1R2, OLAH, | 0.867 | 0.858 | 0.877 | 0.907 | 0.847 | 0.967 | 0.935 | 0.858 | 1 |
| FAM20A | | | | | | | | | |
| SIGLEC1, ISG15, HERC6, SLC1A2, FAM20A, | 0.867 | 0.857 | 0.878 | 0.904 | 0.840 | 0.968 | 0.953 | 0.880 | 1 |
| RETN, MMP8 | | | | | | | | | |
| RSAD2, IFIT1, SIGLEC1, ISG15, SLC1A2, | 0.867 | 0.858 | 0.877 | 0.909 | 0.849 | 0.970 | 0.946 | 0.876 | 1 |
| FAM20A, RETN | | | | | | | | | |
| RSAD2, OAS1, IFI44L, SIGLEC1, ISG15, OLAH, | 0.867 | 0.857 | 0.877 | 0.898 | 0.827 | 0.969 | 0.959 | 0.911 | 1 |
| FAM20A | | | | | | | | | |
| OAS1, IFIT1, SIGLEC1, HERC6, SLC1A2, | 0.867 | 0.857 | 0.877 | 0.915 | 0.860 | 0.969 | 0.940 | 0.887 | 0.993 |
| OLAH, RETN | | | | | | | | | |
| OAS1, IFIT1, SLC1A2, IL1R2, OLAH, FAM20A, | 0.867 | 0.858 | 0.876 | 0.914 | 0.862 | 0.967 | 0.937 | 0.859 | 1 |
| MMP8 | | | | | | | | | |
| IFI27, OAS1, IFI44L, SLC1A2, FAM20A, RETN, | 0.867 | 0.857 | 0.878 | 0.914 | 0.853 | 0.975 | 0.939 | 0.859 | 1 |
| MMP8 | | | | | | | | | |
| IFI27, IFIT1, IFI44L, ISG15, HERC6, SLC1A2, | 0.867 | 0.858 | 0.877 | 0.911 | 0.852 | 0.971 | 0.939 | 0.870 | 1 |
| RETN | | | | | | | | | |
| IFIT1, IFI44L, SIGLEC1, ISG15, HERC6, | 0.867 | 0.858 | 0.877 | 0.913 | 0.858 | 0.968 | 0.940 | 0.888 | 0.993 |
| SLC1A2, OLAH | | | | | | | | | |
| RSAD2, IFIT1, SIGLEC1, HERC6, SLC1A2, | 0.867 | 0.857 | 0.877 | 0.912 | 0.857 | 0.967 | 0.947 | 0.898 | 0.996 |
| OLAH, MMP8 | | | | | | | | | |
| IFIT1, IFI44L, HERC6, OLAH, FAM20A, RETN, | 0.867 | 0.857 | 0.877 | 0.906 | 0.842 | 0.971 | 0.940 | 0.871 | 1 |
| MMP8 | | | | | | | | | |
| SIGLEC1, ISG15, HERC6, SLC1A2, IL1R2, | 0.867 | 0.858 | 0.876 | 0.912 | 0.859 | 0.966 | 0.963 | 0.926 | 1 |
| OLAH, MMP8 | | | | | | | | | |
| IFIT1, IFI44L, SIGLEC1, ISG15, HERC6, IL1R2, | 0.867 | 0.858 | 0.876 | 0.904 | 0.845 | 0.962 | 0.960 | 0.919 | 1 |
| MMP8 | | | | | | | | | |
| IFI27, IFI44L, SIGLEC1, ISG15, SLC1A2, RETN, | 0.867 | 0.858 | 0.877 | 0.914 | 0.856 | 0.971 | 0.951 | 0.893 | 1 |
| MMP8 | | | | | | | | | |
| RSAD2, OAS1, IFIT1, IFI44L, HERC6, IL1R2, | 0.867 | 0.858 | 0.876 | 0.908 | 0.848 | 0.969 | 0.936 | 0.857 | 1 |
| FAM20A | | | | | | | | | |
| IFIT1, IFI44L, SIGLEC1, HERC6, SLC1A2, | 0.867 | 0.857 | 0.877 | 0.914 | 0.858 | 0.969 | 0.939 | 0.886 | 0.992 |
| OLAH, RETN | | | | | | | | | |
| IFI27, OAS1, IFIT1, ISG15, SLC1A2, FAM20A, | 0.867 | 0.857 | 0.877 | 0.908 | 0.847 | 0.969 | 0.939 | 0.858 | 1 |
| RETN | | | | | | | | | |
| RSAD2, IFI27, OAS1, IFI44L, SLC1A2, | 0.867 | 0.857 | 0.877 | 0.910 | 0.848 | 0.972 | 0.940 | 0.861 | 1 |
| FAM20A, RETN | | | | | | | | | |
| RSAD2, IFI27, IFI44L, ISG15, HERC6, SLC1A2, | 0.867 | 0.856 | 0.878 | 0.913 | 0.855 | 0.971 | 0.926 | 0.844 | 1 |
| FAM20A | | | | | | | | | |
| RSAD2, OAS1, ISG15, IL1R2, OLAH, FAM20A, | 0.867 | 0.857 | 0.877 | 0.905 | 0.843 | 0.967 | 0.933 | 0.854 | 1 |
| MMP8 | | | | | | | | | |
| RSAD2, IFI44L, ISG15, HERC6, IL1R2, | 0.867 | 0.857 | 0.877 | 0.912 | 0.854 | 0.970 | 0.930 | 0.847 | 1 |
| FAM20A, MMP8 | | | | | | | | | |
| RSAD2, IFI27, IFI44L, ISG15, SLC1A2, | 0.867 | 0.857 | 0.877 | 0.910 | 0.848 | 0.972 | 0.939 | 0.859 | 1 |
| FAM20A, RETN | | | | | | | | | |
| RSAD2, IFIT1, SLC1A2, IL1R2, OLAH, | 0.867 | 0.858 | 0.876 | 0.914 | 0.861 | 0.966 | 0.936 | 0.857 | 1 |
| FAM20A, MMP8 | | | | | | | | | |
| RSAD2, OAS1, SIGLEC1, ISG15, FAM20A, | 0.867 | 0.857 | 0.877 | 0.904 | 0.837 | 0.971 | 0.955 | 0.893 | 1 |
| RETN, MMP8 | | | | | | | | | |
| OAS1, IFIT1, SIGLEC1, ISG15, HERC6, OLAH, | 0.867 | 0.857 | 0.877 | 0.904 | 0.842 | 0.966 | 0.946 | 0.896 | 0.995 |
| RETN | | | | | | | | | |
| IFIT1, IFI44L, HERC6, IL1R2, FAM20A, RETN, | 0.867 | 0.857 | 0.877 | 0.908 | 0.847 | 0.969 | 0.941 | 0.862 | 1 |
| MMP8 | | | | | | | | | |
| IFIT1, IFI44L, ISG15, SLC1A2, IL1R2, OLAH, | 0.867 | 0.857 | 0.876 | 0.913 | 0.857 | 0.969 | 0.929 | 0.853 | 1 |
| FAM20A | | | | | | | | | |
| IFIT1, SIGLEC1, ISG15, HERC6, SLC1A2, | 0.867 | 0.858 | 0.876 | 0.913 | 0.858 | 0.967 | 0.952 | 0.908 | 0.997 |
| IL1R2, OLAH | | | | | | | | | |
| IFI44L, HERC6, SLC1A2, OLAH, FAM20A, | 0.867 | 0.857 | 0.877 | 0.914 | 0.856 | 0.972 | 0.937 | 0.861 | 1 |
| RETN, MMP8 | | | | | | | | | |
| OAS1, IFIT1, SIGLEC1, HERC6, SLC1A2, | 0.867 | 0.857 | 0.876 | 0.911 | 0.857 | 0.966 | 0.951 | 0.905 | 0.997 |
| IL1R2, RETN | | | | | | | | | |
| IFI27, OAS1, IFI44L, ISG15, SLC1A2, IL1R2, | 0.867 | 0.859 | 0.875 | 0.922 | 0.876 | 0.968 | 0.928 | 0.863 | 0.994 |
| MMP8 | | | | | | | | | |
| OAS1, IFI44L, ISG15, HERC6, IL1R2, FAM20A, | 0.867 | 0.857 | 0.877 | 0.910 | 0.850 | 0.969 | 0.933 | 0.850 | 1 |
| MMP8 | | | | | | | | | |
| RSAD2, OAS1, IFIT1, SIGLEC1, ISG15, HERC6, | 0.867 | 0.857 | 0.877 | 0.903 | 0.839 | 0.967 | 0.933 | 0.875 | 0.990 |
| RETN | | | | | | | | | |
| RSAD2, OAS1, SIGLEC1, HERC6, SLC1A2, | 0.867 | 0.857 | 0.877 | 0.911 | 0.857 | 0.966 | 0.962 | 0.922 | 1 |
| IL1R2, OLAH | | | | | | | | | |
| OAS1, SIGLEC1, ISG15, HERC6, SLC1A2, | 0.867 | 0.857 | 0.877 | 0.911 | 0.857 | 0.966 | 0.959 | 0.917 | 1 |
| IL1R2, OLAH | | | | | | | | | |
| RSAD2, ISG15, HERC6, SLC1A2, IL1R2, OLAH, | 0.867 | 0.857 | 0.877 | 0.913 | 0.85° | 0.967 | 0.927 | 0.842 | 1 |
| FAM20A | | | | | | | | | |
| OAS1, SIGLEC1, HERC6, SLC1A2, IL1R2, | 0.867 | 0.858 | 0.876 | 0.914 | 0.859 | 0.968 | 0.967 | 0.932 | 1 |
| RETN, MMP8 | | | | | | | | | |
| IFIT1, IFI44L, HERC6, SLC1A2, IL1R2, | 0.867 | 0.857 | 0.877 | 0.916 | 0.865 | 0.967 | 0.924 | 0.830 | 1 |
| FAM20A, MMP8 | | | | | | | | | |
| IFI27, IFIT1, IFI44L, SIGLEC1, SLC1A2, RETN, | 0.867 | 0.858 | 0.876 | 0.916 | 0.860 | 0.972 | 0.952 | 0.894 | 1 |
| MMP8 | | | | | | | | | |
| RSAD2, IFI44L, SIGLEC1, HERC6, OLAH, | 0.867 | 0.857 | 0.877 | 0.907 | 0.845 | 0.970 | 0.946 | 0.888 | 1 |
| FAM20A, RETN | | | | | | | | | |
| IFI27, SIGLEC1, ISG15, HERC6, SLC1A2, | 0.867 | 0.857 | 0.877 | 0.911 | 0.849 | 0.972 | 0.950 | 0.891 | 1 |
| RETN, MMP8 | | | | | | | | | |
| OAS1, IFI44L, HERC6, SLC1A2, OLAH, | 0.867 | 0.856 | 0.878 | 0.914 | 0.859 | 0.969 | 0.927 | 0.850 | 1 |
| FAM20A, MMP8 | | | | | | | | | |
| OAS1, IFIT1, SIGLEC1, HERC6, SLC1A2, | 0.867 | 0.857 | 0.877 | 0.913 | 0.859 | 0.967 | 0.953 | 0.909 | 0.997 |
| IL1R2, OLAH | | | | | | | | | |
| RSAD2, OAS1, IFI44L, SLC1A2, OLAH, | 0.867 | 0.857 | 0.877 | 0.915 | 0.859 | 0.971 | 0.924 | 0.849 | 0.999 |
| FAM20A, MMP8 | | | | | | | | | |
| RSAD2, IFIT1, SIGLEC1, HERC6, SLC1A2, | 0.867 | 0.857 | 0.876 | 0.914 | 0.859 | 0.968 | 0.954 | 0.911 | 0.998 |
| IL1R2, OLAH | | | | | | | | | |
| OAS1, SIGLEC1, HERC6, IL1R2, OLAH, RETN, | 0.867 | 0.857 | 0.877 | 0.904 | 0.841 | 0.968 | 0.967 | 0.932 | 1 |
| MMP8 | | | | | | | | | |
| RSAD2, IFI27, IFI44L, SLC1A2, FAM20A, | 0.867 | 0.856 | 0.877 | 0.914 | 0.854 | 0.975 | 0.940 | 0.861 | 1 |
| RETN, MMP8 | | | | | | | | | |
| OAS1, IFI44L, SLC1A2, IL1R2, OLAH, | 0.867 | 0.857 | 0.877 | 0.917 | 0.865 | 0.969 | 0.929 | 0.848 | 1 |
| FAM20A, MMP8 | | | | | | | | | |
| OAS1, IFIT1, IFI44L, SIGLEC1, ISG15, HERC6, | 0.867 | 0.857 | 0.876 | 0.904 | 0.841 | 0.967 | 0.932 | 0.874 | 0.989 |
| RETN | | | | | | | | | |
| OAS1, IFI44L, ISG15, HERC6, SLC1A2, OLAH, | 0.867 | 0.856 | 0.877 | 0.911 | 0.855 | 0.968 | 0.925 | 0.845 | 1 |
| FAM20A | | | | | | | | | |
| IFI44L, SIGLEC1, SLC1A2, IL1R2, OLAH, | 0.867 | 0.857 | 0.876 | 0.914 | 0.859 | 0.968 | 0.970 | 0.937 | 1 |
| RETN, MMP8 | | | | | | | | | |
| IFIT1, IFI44L, SIGLEC1, SLC1A2, OLAH, RETN, | 0.867 | 0.857 | 0.876 | 0.913 | 0.857 | 0.969 | 0.955 | 0.911 | 1 |
| MMP8 | | | | | | | | | |
| OAS1, IFIT1, SIGLEC1, HERC6, IL1R2, OLAH, | 0.867 | 0.858 | 0.876 | 0.905 | 0.848 | 0.963 | 0.958 | 0.915 | 1.000 |
| MMP8 | | | | | | | | | |
| OAS1, IFIT1, IFI44L, SIGLEC1, SLC1A2, OLAH, | 0.867 | 0.857 | 0.876 | 0.911 | 0.855 | 0.967 | 0.952 | 0.907 | 0.998 |
| MMP8 | | | | | | | | | |
| RSAD2, IFI44L, ISG15, HERC6, IL1R2, | 0.867 | 0.857 | 0.877 | 0.911 | 0.852 | 0.969 | 0.936 | 0.860 | 1 |
| FAM20A, RETN | | | | | | | | | |
| IFIT1, SIGLEC1, HERC6, SLC1A2, IL1R2, | 0.867 | 0.857 | 0.876 | 0.911 | 0.857 | 0.966 | 0.963 | 0.925 | 1 |
| RETN, MMP8 | | | | | | | | | |
| RSAD2, IFI27, IFI44L, HERC6, SLC1A2, RETN, | 0.867 | 0.857 | 0.876 | 0.912 | 0.853 | 0.971 | 0.933 | 0.864 | 1 |
| MMP8 | | | | | | | | | |
| RSAD2, OAS1, SLC1A2, IL1R2, OLAH, | 0.867 | 0.858 | 0.876 | 0.914 | 0.861 | 0.967 | 0.932 | 0.849 | 1 |
| FAM20A, MMP8 | | | | | | | | | |
| RSAD2, IFI27, IFI44L, HERC6, SLC1A2, | 0.867 | 0.856 | 0.877 | 0.913 | 0.857 | 0.970 | 0.925 | 0.842 | 1 |
| FAM20A, MMP8 | | | | | | | | | |
| IFIT1, IFI44L, SLC1A2, OLAH, FAM20A, RETN, | 0.867 | 0.857 | 0.876 | 0.913 | 0.856 | 0.970 | 0.927 | 0.852 | 1 |
| MMP8 | | | | | | | | | |
| OAS1, IFIT1, IFI44L, SLC1A2, OLAH, FAM20A, | 0.867 | 0.857 | 0.877 | 0.913 | 0.856 | 0.969 | 0.925 | 0.849 | 1 |
| MMP8 | | | | | | | | | |
| OAS1, SIGLEC1, ISG15, HERC6, SLC1A2, | 0.867 | 0.857 | 0.877 | 0.911 | 0.855 | 0.966 | 0.954 | 0.910 | 0.999 |
| OLAH, MMP8 | | | | | | | | | |
| RSAD2, IFI44L, SIGLEC1, ISG15, HERC6, | 0.867 | 0.857 | 0.877 | 0.910 | 0.853 | 0.967 | 0.939 | 0.863 | 1 |
| SLC1A2, FAM20A | | | | | | | | | |
| RSAD2, IFI44L, SIGLEC1, OLAH, FAM20A, | 0.867 | 0.856 | 0.877 | 0.908 | 0.844 | 0.972 | 0.962 | 0.919 | 1 |
| RETN, MMP8 | | | | | | | | | |
| RSAD2, OAS1, IFIT1, IFI44L, SIGLEC1, HERC6, | 0.867 | 0.858 | 0.876 | 0.901 | 0.841 | 0.962 | 0.961 | 0.921 | 1 |
| IL1R2 | | | | | | | | | |
| IFI44L, SIGLEC1, HERC6, IL1R2, OLAH, RETN, | 0.867 | 0.857 | 0.877 | 0.902 | 0.838 | 0.966 | 0.962 | 0.922 | 1 |
| MMP8 | | | | | | | | | |
| RSAD2, IFI27, SIGLEC1, ISG15, SLC1A2, | 0.867 | 0.857 | 0.876 | 0.914 | 0.856 | 0.971 | 0.952 | 0.895 | 1 |
| RETN, MMP8 | | | | | | | | | |
| RSAD2, OAS1, SIGLEC1, HERC6, SLC1A2, | 0.867 | 0.856 | 0.877 | 0.912 | 0.855 | 0.968 | 0.942 | 0.870 | 1 |
| FAM20A, RETN | | | | | | | | | |
| IFIT1, IFI44L, ISG15, HERC6, OLAH, FAM20A, | 0.867 | 0.857 | 0.877 | 0.901 | 0.838 | 0.965 | 0.937 | 0.868 | 1 |
| MMP8 | | | | | | | | | |
| IFIT1, ISG15, HERC6, SLC1A2, IL1R2, OLAH, | 0.867 | 0.857 | 0.876 | 0.912 | 0.858 | 0.967 | 0.928 | 0.844 | 1 |
| FAM20A | | | | | | | | | |
| OAS1, IFI44L, ISG15, HERC6, IL1R2, FAM20A, | 0.867 | 0.856 | 0.877 | 0.908 | 0.848 | 0.968 | 0.939 | 0.864 | 1 |
| RETN | | | | | | | | | |
| RSAD2, OAS1, IFI44L, SIGLEC1, ISG15, | 0.867 | 0.857 | 0.876 | 0.902 | 0.838 | 0.967 | 0.940 | 0.869 | 1 |
| HERC6, FAM20A | | | | | | | | | |
| IFI44L, SIGLEC1, ISG15, HERC6, SLC1A2, | 0.866 | 0.856 | 0.877 | 0.904 | 0.841 | 0.966 | 0.950 | 0.876 | 1 |
| FAM20A, RETN | | | | | | | | | |
| RSAD2, OAS1, SIGLEC1, HERC6, SLC1A2, | 0.866 | 0.856 | 0.877 | 0.911 | 0.856 | 0.967 | 0.954 | 0.910 | 0.999 |
| OLAH, MMP8 | | | | | | | | | |
| OAS1, IFI44L, HERC6, SLC1A2, IL1R2, OLAH, | 0.866 | 0.856 | 0.877 | 0.914 | 0.860 | 0.967 | 0.923 | 0.838 | 1 |
| FAM20A | | | | | | | | | |
| RSAD2, IFIT1, IFI44L, HERC6, IL1R2, OLAH, | 0.866 | 0.857 | 0.876 | 0.908 | 0.849 | 0.967 | 0.933 | 0.855 | 1 |
| FAM20A | | | | | | | | | |
| IFI27, IFI44L, ISG15, HERC6, SLC1A2, RETN, | 0.866 | 0.857 | 0.876 | 0.909 | 0.848 | 0.970 | 0.934 | 0.862 | 1 |
| MMP8 | | | | | | | | | |
| IFI44L, SIGLEC1, HERC6, SLC1A2, IL1R2, | 0.866 | 0.857 | 0.876 | 0.911 | 0.857 | 0.965 | 0.965 | 0.929 | 1 |
| OLAH, MMP8 | | | | | | | | | |
| IFI44L, HERC6, SLC1A2, IL1R2, OLAH, | 0.866 | 0.856 | 0.877 | 0.914 | 0.860 | 0.968 | 0.929 | 0.846 | 1 |
| FAM20A, RETN | | | | | | | | | |
| OAS1, IFIT1, SIGLEC1, HERC6, IL1R2, OLAH, | 0.866 | 0.857 | 0.876 | 0.905 | 0.843 | 0.967 | 0.945 | 0.895 | 0.994 |
| RETN | | | | | | | | | |
| RSAD2, IFIT1, IFI44L, SIGLEC1, HERC6, IL1R2, | 0.866 | 0.858 | 0.875 | 0.903 | 0.844 | 0.963 | 0.959 | 0.917 | 1 |
| MMP8 | | | | | | | | | |
| OAS1, SIGLEC1, ISG15, HERC6, SLC1A2, | 0.866 | 0.857 | 0.876 | 0.909 | 0.853 | 0.965 | 0.958 | 0.912 | 1 |
| IL1R2, RETN | | | | | | | | | |
| IFIT1, IFI44L, SIGLEC1, ISG15, HERC6, OLAH, | 0.866 | 0.857 | 0.876 | 0.902 | 0.837 | 0.967 | 0.948 | 0.900 | 0.995 |
| RETN | | | | | | | | | |
| IFIT1, SIGLEC1, ISG15, HERC6, IL1R2, OLAH, | 0.866 | 0.857 | 0.876 | 0.905 | 0.844 | 0.967 | 0.950 | 0.904 | 0.996 |
| RETN | | | | | | | | | |
| RSAD2, OAS1, IFIT1, SIGLEC1, SLC1A2, | 0.866 | 0.857 | 0.876 | 0.911 | 0.855 | 0.967 | 0.957 | 0.913 | 1.000 |
| OLAH, MMP8 | | | | | | | | | |
| IFIT1, SIGLEC1, ISG15, HERC6, SLC1A2, | 0.866 | 0.856 | 0.876 | 0.912 | 0.856 | 0.968 | 0.945 | 0.895 | 0.994 |
| OLAH, RETN | | | | | | | | | |
| OAS1, IFIT1, IFI44L, SIGLEC1, HERC6, OLAH, | 0.866 | 0.857 | 0.876 | 0.901 | 0.836 | 0.965 | 0.938 | 0.884 | 0.992 |
| RETN | | | | | | | | | |
| OAS1, IFIT1, IFI44L, SIGLEC1, ISG15, HERC6, | 0.866 | 0.857 | 0.875 | 0.904 | 0.847 | 0.962 | 0.954 | 0.909 | 0.999 |
| IL1R2 | | | | | | | | | |
| RSAD2, OAS1, SIGLEC1, ISG15, HERC6, IL1R2, | 0.866 | 0.856 | 0.876 | 0.903 | 0.842 | 0.965 | 0.962 | 0.921 | 1 |
| RETN | | | | | | | | | |
| IFIT1, ISG15, SLC1A2, IL1R2, OLAH, FAM20A, | 0.866 | 0.857 | 0.876 | 0.913 | 0.859 | 0.966 | 0.934 | 0.855 | 1 |
| MMP8 | | | | | | | | | |
| RSAD2, OAS1, IFIT1, IFI44L, IL1R2, FAM20A, | 0.866 | 0.857 | 0.876 | 0.909 | 0.848 | 0.970 | 0.942 | 0.867 | 1 |
| MMP8 | | | | | | | | | |
| IFI27, OAS1, IFI44L, SIGLEC1, HERC6, | 0.866 | 0.857 | 0.875 | 0.911 | 0.857 | 0.964 | 0.921 | 0.850 | 0.992 |
| SLC1A2, MMP8 | | | | | | | | | |
| IFIT1, IFI44L, HERC6, IL1R2, OLAH, FAM20A, | 0.866 | 0.857 | 0.876 | 0.905 | 0.844 | 0.967 | 0.934 | 0.857 | 1 |
| RETN | | | | | | | | | |
| RSAD2, IFI27, OAS1, SIGLEC1, ISG15, HERC6, | 0.866 | 0.857 | 0.875 | 0.908 | 0.852 | 0.963 | 0.917 | 0.846 | 0.989 |
| MMP8 | | | | | | | | | |
| RSAD2, OAS1, IFIT1, SIGLEC1, HERC6, IL1R2, | 0.866 | 0.857 | 0.876 | 0.905 | 0.848 | 0.962 | 0.961 | 0.921 | 1 |
| MMP8 | | | | | | | | | |
| OAS1, IFIT1, HERC6, SLC1A2, IL1R2, FAM20A, | 0.866 | 0.857 | 0.876 | 0.916 | 0.863 | 0.968 | 0.929 | 0.842 | 1 |
| MMP8 | | | | | | | | | |
| RSAD2, OAS1, HERC6, IL1R2, OLAH, FAM20A, | 0.866 | 0.856 | 0.877 | 0.906 | 0.845 | 0.967 | 0.937 | 0.863 | 1 |
| RETN | | | | | | | | | |
| RSAD2, OAS1, ISG15, HERC6, SLC1A2, IL1R2, | 0.866 | 0.856 | 0.876 | 0.910 | 0.857 | 0.963 | 0.925 | 0.833 | 1 |
| FAM20A | | | | | | | | | |
| RSAD2, IFI27, IFIT1, IFI44L, HERC6, SLC1A2, | 0.866 | 0.856 | 0.877 | 0.914 | 0.858 | 0.970 | 0.923 | 0.839 | 1 |
| FAM20A | | | | | | | | | |
| RSAD2, IFI27, OAS1, IFIT1, ISG15, HERC6, | 0.866 | 0.856 | 0.877 | 0.907 | 0.846 | 0.967 | 0.930 | 0.849 | 1 |
| FAM20A | | | | | | | | | |
| RSAD2, OAS1, IFI44L, SIGLEC1, HERC6, | 0.866 | 0.856 | 0.876 | 0.910 | 0.853 | 0.966 | 0.938 | 0.862 | 1 |
| SLC1A2, FAM20A | | | | | | | | | |
| OAS1, IFIT1, SIGLEC1, SLC1A2, IL1R2, OLAH, | 0.866 | 0.857 | 0.875 | 0.912 | 0.857 | 0.966 | 0.966 | 0.931 | 1 |
| MMP8 | | | | | | | | | |
| RSAD2, IFIT1, SIGLEC1, SLC1A2, IL1R2, | 0.866 | 0.857 | 0.875 | 0.912 | 0.858 | 0.967 | 0.965 | 0.929 | 1 |
| OLAH, MMP8 | | | | | | | | | |
| RSAD2, SIGLEC1, HERC6, IL1R2, OLAH, | 0.866 | 0.856 | 0.876 | 0.906 | 0.844 | 0.968 | 0.962 | 0.923 | 1 |
| RETN, MMP8 | | | | | | | | | |
| OAS1, IFIT1, SIGLEC1, ISG15, HERC6, | 0.866 | 0.856 | 0.876 | 0.913 | 0.858 | 0.968 | 0.946 | 0.897 | 0.995 |
| SLC1A2, OLAH | | | | | | | | | |
| IFIT1, IFI44L, SIGLEC1, HERC6, IL1R2, OLAH, | 0.866 | 0.857 | 0.875 | 0.907 | 0.849 | 0.964 | 0.963 | 0.924 | 1 |
| MMP8 | | | | | | | | | |
| RSAD2, IFI27, IFIT1, HERC6, SLC1A2, RETN, | 0.866 | 0.857 | 0.876 | 0.912 | 0.853 | 0.971 | 0.934 | 0.865 | 1 |
| MMP8 | | | | | | | | | |
| OAS1, IFI44L, ISG15, HERC6, SLC1A2, IL1R2, | 0.866 | 0.856 | 0.877 | 0.914 | 0.862 | 0.965 | 0.926 | 0.836 | 1 |
| FAM20A | | | | | | | | | |
| RSAD2, IFI44L, ISG15, SLC1A2, OLAH, | 0.866 | 0.856 | 0.876 | 0.912 | 0.854 | 0.970 | 0.922 | 0.845 | 0.998 |
| FAM20A, MMP8 | | | | | | | | | |
| IFI44L, SIGLEC1, ISG15, SLC1A2, IL1R2, | 0.866 | 0.857 | 0.875 | 0.914 | 0.859 | 0.969 | 0.962 | 0.922 | 1 |
| OLAH, RETN | | | | | | | | | |
| RSAD2, SIGLEC1, ISG15, HERC6, SLC1A2, | 0.866 | 0.857 | 0.876 | 0.912 | 0.857 | 0.966 | 0.955 | 0.912 | 0.999 |
| IL1R2, OLAH | | | | | | | | | |
| OAS1, IFI44L, SIGLEC1, HERC6, IL1R2, OLAH, | 0.866 | 0.857 | 0.875 | 0.903 | 0.844 | 0.962 | 0.963 | 0.924 | 1 |
| MMP8 | | | | | | | | | |
| RSAD2, OAS1, SIGLEC1, SLC1A2, IL1R2, | 0.866 | 0.856 | 0.876 | 0.912 | 0.858 | 0.966 | 0.967 | 0.932 | 1 |
| OLAH, MMP8 | | | | | | | | | |
| RSAD2, SIGLEC1, ISG15, SLC1A2, IL1R2, | 0.866 | 0.857 | 0.875 | 0.912 | 0.858 | 0.966 | 0.965 | 0.929 | 1 |
| OLAH, MMP8 | | | | | | | | | |
| IFIT1, SIGLEC1, ISG15, HERC6, IL1R2, OLAH, | 0.866 | 0.857 | 0.875 | 0.907 | 0.850 | 0.964 | 0.961 | 0.921 | 1 |
| MMP8 | | | | | | | | | |
| OAS1, IFIT1, IFI44L, SIGLEC1, HERC6, IL1R2, | 0.866 | 0.857 | 0.875 | 0.905 | 0.846 | 0.964 | 0.952 | 0.907 | 0.997 |
| OLAH | | | | | | | | | |
| RSAD2, IFIT1, SIGLEC1, ISG15, HERC6, IL1R2, | 0.866 | 0.857 | 0.875 | 0.905 | 0.843 | 0.967 | 0.952 | 0.907 | 0.998 |
| RETN | | | | | | | | | |
| RSAD2, IFI44L, SIGLEC1, HERC6, IL1R2, | 0.866 | 0.856 | 0.876 | 0.901 | 0.837 | 0.965 | 0.963 | 0.925 | 1 |
| RETN, MMP8 | | | | | | | | | |
| OAS1, IFIT1, SIGLEC1, ISG15, HERC6, IL1R2, | 0.866 | 0.857 | 0.875 | 0.906 | 0.850 | 0.962 | 0.958 | 0.914 | 1 |
| MMP8 | | | | | | | | | |
| IFIT1, IFI44L, ISG15, HERC6, SLC1A2, IL1R2, | 0.866 | 0.856 | 0.876 | 0.915 | 0.864 | 0.966 | 0.926 | 0.835 | 1 |
| FAM20A | | | | | | | | | |
| RSAD2, OAS1, SIGLEC1, SLC1A2, FAM20A, | 0.866 | 0.856 | 0.876 | 0.909 | 0.849 | 0.969 | 0.955 | 0.888 | 1 |
| RETN, MMP8 | | | | | | | | | |
| IFIT1, IFI44L, SIGLEC1, SLC1A2, IL1R2, OLAH, | 0.866 | 0.857 | 0.875 | 0.912 | 0.858 | 0.966 | 0.966 | 0.931 | 1 |
| MMP8 | | | | | | | | | |
| RSAD2, OAS1, IFI44L, SIGLEC1, HERC6, | 0.866 | 0.857 | 0.875 | 0.902 | 0.843 | 0.962 | 0.962 | 0.922 | 1 |
| IL1R2, MMP8 | | | | | | | | | |
| SIGLEC1, ISG15, SLC1A2, IL1R2, OLAH, | 0.866 | 0.857 | 0.875 | 0.912 | 0.857 | 0.967 | 0.966 | 0.931 | 1 |
| RETN, MMP8 | | | | | | | | | |
| IFI44L, ISG15, HERC6, SLC1A2, IL1R2, | 0.866 | 0.856 | 0.876 | 0.917 | 0.867 | 0.967 | 0.924 | 0.830 | 1 |
| FAM20A, MMP8 | | | | | | | | | |
| OAS1, IFIT1, IFI44L, IL1R2, OLAH, FAM20A, | 0.866 | 0.857 | 0.875 | 0.905 | 0.843 | 0.967 | 0.945 | 0.875 | 1 |
| MMP8 | | | | | | | | | |
| RSAD2, OAS1, IFIT1, SIGLEC1, SLC1A2, | 0.866 | 0.857 | 0.875 | 0.913 | 0.857 | 0.968 | 0.967 | 0.931 | 1 |
| IL1R2, OLAH | | | | | | | | | |
| IFI44L, SIGLEC1, ISG15, SLC1A2, FAM20A, | 0.866 | 0.856 | 0.876 | 0.904 | 0.841 | 0.967 | 0.953 | 0.882 | 1 |
| RETN, MMP8 | | | | | | | | | |
| RSAD2, IFI44L, ISG15, HERC6, SLC1A2, IL1R2, | 0.866 | 0.856 | 0.876 | 0.914 | 0.862 | 0.966 | 0.926 | 0.837 | 1 |
| FAM20A | | | | | | | | | |
| ISG15, HERC6, SLC1A2, IL1R2, FAM20A, | 0.866 | 0.856 | 0.875 | 0.917 | 0.864 | 0.970 | 0.927 | 0.838 | 1 |
| RETN, MMP8 | | | | | | | | | |
| RSAD2, OAS1, SIGLEC1, ISG15, OLAH, | 0.866 | 0.856 | 0.876 | 0.896 | 0.822 | 0.969 | 0.960 | 0.914 | 1 |
| FAM20A, RETN | | | | | | | | | |
| OAS1, IFIT1, SIGLEC1, ISG15, SLC1A2, OLAH, | 0.866 | 0.856 | 0.875 | 0.912 | 0.857 | 0.968 | 0.951 | 0.906 | 0.997 |
| MMP8 | | | | | | | | | |
| OAS1, IFIT1, ISG15, HERC6, SLC1A2, IL1R2, | 0.866 | 0.856 | 0.876 | 0.911 | 0.857 | 0.965 | 0.928 | 0.842 | 1 |
| FAM20A | | | | | | | | | |
| RSAD2, IFIT1, IFI44L, SIGLEC1, SLC1A2, | 0.866 | 0.857 | 0.875 | 0.911 | 0.855 | 0.967 | 0.954 | 0.910 | 0.998 |
| OLAH, MMP8 | | | | | | | | | |
| RSAD2, OAS1, SIGLEC1, HERC6, IL1R2, | 0.866 | 0.856 | 0.876 | 0.904 | 0.847 | 0.962 | 0.961 | 0.920 | 1 |
| OLAH, MMP8 | | | | | | | | | |
| IFIT1, IFI44L, SIGLEC1, ISG15, SLC1A2, OLAH, | 0.866 | 0.857 | 0.875 | 0.915 | 0.860 | 0.971 | 0.940 | 0.885 | 0.995 |
| RETN | | | | | | | | | |
| RSAD2, OAS1, IFI44L, SIGLEC1, HERC6, | 0.866 | 0.856 | 0.875 | 0.907 | 0.844 | 0.970 | 0.943 | 0.876 | 1 |
| FAM20A, RETN | | | | | | | | | |
| IFIT1, SIGLEC1, SLC1A2, IL1R2, OLAH, RETN, | 0.866 | 0.856 | 0.875 | 0.915 | 0.861 | 0.968 | 0.967 | 0.933 | 1 |
| MMP8 | | | | | | | | | |
| RSAD2, OAS1, IFI44L, HERC6, SLC1A2, OLAH, | 0.866 | 0.855 | 0.876 | 0.912 | 0.856 | 0.968 | 0.924 | 0.845 | 1 |
| FAM20A | | | | | | | | | |
| RSAD2, IFIT1, IFI44L, SIGLEC1, HERC6, IL1R2, | 0.866 | 0.857 | 0.875 | 0.904 | 0.842 | 0.967 | 0.953 | 0.909 | 0.997 |
| RETN | | | | | | | | | |
| OAS1, IFI44L, ISG15, SLC1A2, OLAH, | 0.866 | 0.855 | 0.876 | 0.911 | 0.853 | 0.969 | 0.925 | 0.849 | 1 |
| FAM20A, MMP8 | | | | | | | | | |
| OAS1, IFIT1, SIGLEC1, ISG15, SLC1A2, IL1R2, | 0.866 | 0.856 | 0.875 | 0.912 | 0.857 | 0.967 | 0.961 | 0.922 | 1 |
| OLAH | | | | | | | | | |
| RSAD2, OAS1, IFIT1, SIGLEC1, HERC6, IL1R2, | 0.866 | 0.856 | 0.875 | 0.906 | 0.848 | 0.964 | 0.954 | 0.910 | 0.998 |
| OLAH | | | | | | | | | |
| IFIT1, SIGLEC1, HERC6, SLC1A2, IL1R2, | 0.866 | 0.856 | 0.876 | 0.916 | 0.862 | 0.969 | 0.949 | 0.902 | 0.996 |
| OLAH, RETN | | | | | | | | | |
| OAS1, IFI44L, HERC6, IL1R2, FAM20A, RETN, | 0.866 | 0.856 | 0.875 | 0.909 | 0.848 | 0.970 | 0.938 | 0.857 | 1 |
| MMP8 | | | | | | | | | |
| RSAD2, IFIT1, HERC6, SLC1A2, IL1R2, | 0.866 | 0.856 | 0.875 | 0.915 | 0.863 | 0.967 | 0.929 | 0.842 | 1 |
| FAM20A, MMP8 | | | | | | | | | |
| RSAD2, IFIT1, IFI44L, SIGLEC1, HERC6, | 0.866 | 0.856 | 0.875 | 0.912 | 0.857 | 0.967 | 0.940 | 0.888 | 0.993 |
| SLC1A2, OLAH | | | | | | | | | |
| RSAD2, OAS1, IFI44L, SIGLEC1, HERC6, | 0.866 | 0.856 | 0.875 | 0.900 | 0.835 | 0.964 | 0.957 | 0.913 | 1 |
| IL1R2, RETN | | | | | | | | | |
| OAS1, SIGLEC1, ISG15, SLC1A2, IL1R2, OLAH, | 0.866 | 0.856 | 0.875 | 0.913 | 0.859 | 0.966 | 0.965 | 0.929 | 1 |
| MMP8 | | | | | | | | | |
| OAS1, IFIT1, HERC6, IL1R2, OLAH, FAM20A, | 0.866 | 0.856 | 0.876 | 0.905 | 0.844 | 0.967 | 0.937 | 0.863 | 1 |
| RETN | | | | | | | | | |
| RSAD2, OAS1, SIGLEC1, HERC6, SLC1A2, | 0.866 | 0.856 | 0.875 | 0.912 | 0.857 | 0.967 | 0.962 | 0.919 | 1 |
| IL1R2, RETN | | | | | | | | | |
| RSAD2, IFIT1, IFI44L, HERC6, IL1R2, FAM20A, | 0.866 | 0.856 | 0.875 | 0.908 | 0.848 | 0.968 | 0.935 | 0.853 | 1 |
| MMP8 | | | | | | | | | |
| IFIT1, HERC6, SLC1A2, IL1R2, FAM20A, | 0.866 | 0.856 | 0.875 | 0.915 | 0.860 | 0.969 | 0.930 | 0.845 | 1 |
| RETN, MMP8 | | | | | | | | | |
| RSAD2, IFI44L, ISG15, HERC6, SLC1A2, OLAH, | 0.866 | 0.855 | 0.876 | 0.913 | 0.858 | 0.969 | 0.925 | 0.844 | 1 |
| FAM20A | | | | | | | | | |
| RSAD2, IFI27, IFI44L, SIGLEC1, SLC1A2, | 0.866 | 0.856 | 0.875 | 0.915 | 0.859 | 0.97 | 0.954 | 0.899 | 1 |
| RETN, MMP8 | | | | | | | | | |
| OAS1, IFIT1, ISG15, IL1R2, OLAH, FAM20A, | 0.866 | 0.856 | 0.875 | 0.907 | 0.845 | 0.969 | 0.943 | 0.871 | 1 |
| MMP8 | | | | | | | | | |
| OAS1, IFIT1, SIGLEC1, HERC6, SLC1A2, | 0.866 | 0.856 | 0.875 | 0.910 | 0.858 | 0.963 | 0.964 | 0.927 | 1 |
| IL1R2, MMP8 | | | | | | | | | |
| IFI27, OAS1, IFI44L, HERC6, SLC1A2, | 0.866 | 0.855 | 0.876 | 0.912 | 0.856 | 0.969 | 0.926 | 0.844 | 1 |
| FAM20A, MMP8 | | | | | | | | | |
| OAS1, IFIT1, SIGLEC1, SLC1A2, IL1R2, OLAH, | 0.866 | 0.856 | 0.875 | 0.913 | 0.858 | 0.968 | 0.958 | 0.915 | 1 |
| RETN | | | | | | | | | |
| ISG15, HERC6, SLC1A2, IL1R2, OLAH, | 0.866 | 0.856 | 0.875 | 0.915 | 0.865 | 0.966 | 0.926 | 0.839 | 1 |
| FAM20A, MMP8 | | | | | | | | | |
| IFI44L, SIGLEC1, ISG15, HERC6, IL1R2, OLAH, | 0.866 | 0.857 | 0.875 | 0.903 | 0.844 | 0.962 | 0.963 | 0.924 | 1 |
| MMP8 | | | | | | | | | |
| RSAD2, IFIT1, IFI44L, SIGLEC1, ISG15, HERC6, | 0.866 | 0.857 | 0.874 | 0.901 | 0.840 | 0.962 | 0.952 | 0.907 | 0.997 |
| IL1R2 | | | | | | | | | |
| RSAD2, IFI27, OAS1, ISG15, SLC1A2, FAM20A, | 0.866 | 0.855 | 0.876 | 0.911 | 0.850 | 0.972 | 0.941 | 0.861 | 1 |
| RETN | | | | | | | | | |
| OAS1, IFIT1, SIGLEC1, SLC1A2, OLAH, RETN, | 0.866 | 0.856 | 0.875 | 0.912 | 0.856 | 0.968 | 0.959 | 0.917 | 1 |
| MMP8 | | | | | | | | | |
| RSAD2, SIGLEC1, HERC6, SLC1A2, IL1R2, | 0.866 | 0.855 | 0.876 | 0.914 | 0.860 | 0.968 | 0.955 | 0.912 | 0.999 |
| OLAH, RETN | | | | | | | | | |
| SIGLEC1, HERC6, SLC1A2, IL1R2, OLAH, | 0.865 | 0.856 | 0.875 | 0.913 | 0.858 | 0.968 | 0.967 | 0.933 | 1 |
| RETN, MMP8 | | | | | | | | | |
| OAS1, IFI44L, SIGLEC1, ISG15, HERC6, IL1R2, | 0.865 | 0.856 | 0.875 | 0.904 | 0.843 | 0.965 | 0.954 | 0.909 | 1.000 |
| RETN | | | | | | | | | |
| OAS1, ISG15, HERC6, SLC1A2, IL1R2, | 0.865 | 0.855 | 0.876 | 0.912 | 0.857 | 0.966 | 0.927 | 0.839 | 1 |
| FAM20A, RETN | | | | | | | | | |
| RSAD2, IFIT1, SIGLEC1, HERC6, SLC1A2, | 0.865 | 0.856 | 0.875 | 0.912 | 0.856 | 0.968 | 0.943 | 0.893 | 0.994 |
| OLAH, RETN | | | | | | | | | |
| IFI27, OAS1, IFIT1, SLC1A2, FAM20A, RETN, | 0.865 | 0.855 | 0.876 | 0.908 | 0.845 | 0.971 | 0.941 | 0.861 | 1 |
| MMP8 | | | | | | | | | |
| RSAD2, OAS1, IL1R2, OLAH, FAM20A, RETN, | 0.865 | 0.855 | 0.875 | 0.907 | 0.845 | 0.968 | 0.938 | 0.861 | 1 |
| MMP8 | | | | | | | | | |
| RSAD2, IFI27, IFIT1, ISG15, SLC1A2, FAM20A, | 0.865 | 0.855 | 0.876 | 0.908 | 0.847 | 0.969 | 0.940 | 0.859 | 1 |
| RETN | | | | | | | | | |
| RSAD2, IFIT1, HERC6, IL1R2, FAM20A, RETN, | 0.865 | 0.856 | 0.875 | 0.906 | 0.844 | 0.969 | 0.941 | 0.863 | 1 |
| MMP8 | | | | | | | | | |
| IFI44L, SIGLEC1, ISG15, HERC6, SLC1A2, | 0.865 | 0.856 | 0.875 | 0.911 | 0.856 | 0.967 | 0.951 | 0.905 | 0.997 |
| OLAH, MMP8 | | | | | | | | | |
| RSAD2, IFIT1, SIGLEC1, ISG15, SLC1A2, | 0.865 | 0.856 | 0.875 | 0.912 | 0.857 | 0.968 | 0.952 | 0.907 | 0.997 |
| OLAH, MMP8 | | | | | | | | | |
| RSAD2, IFIT1, SIGLEC1, ISG15, HERC6, | 0.865 | 0.856 | 0.875 | 0.913 | 0.858 | 0.968 | 0.945 | 0.895 | 0.994 |
| SLC1A2, OLAH | | | | | | | | | |
| RSAD2, IFI44L, HERC6, SLC1A2, IL1R2, | 0.865 | 0.856 | 0.875 | 0.919 | 0.869 | 0.968 | 0.924 | 0.830 | 1 |
| FAM20A, MMP8 | | | | | | | | | |
| OAS1, IFI44L, HERC6, SLC1A2, IL1R2, | 0.865 | 0.855 | 0.875 | 0.916 | 0.866 | 0.966 | 0.923 | 0.827 | 1 |
| FAM20A, MMP8 | | | | | | | | | |
| IFIT1, IFI44L, SIGLEC1, ISG15, HERC6, IL1R2, | 0.865 | 0.856 | 0.874 | 0.906 | 0.848 | 0.964 | 0.954 | 0.910 | 0.998 |
| OLAH | | | | | | | | | |
| RSAD2, SIGLEC1, HERC6, SLC1A2, IL1R2, | 0.865 | 0.856 | 0.875 | 0.910 | 0.854 | 0.965 | 0.964 | 0.926 | 1 |
| RETN, MMP8 | | | | | | | | | |
| OAS1, IFIT1, IFI44L, SIGLEC1, SLC1A2, OLAH, | 0.865 | 0.856 | 0.875 | 0.915 | 0.860 | 0.971 | 0.940 | 0.885 | 0.995 |
| RETN | | | | | | | | | |
| ISG15, HERC6, SLC1A2, IL1R2, OLAH, | 0.865 | 0.855 | 0.876 | 0.913 | 0.858 | 0.968 | 0.926 | 0.840 | 1 |
| FAM20A, RETN | | | | | | | | | |
| OAS1, SIGLEC1, ISG15, HERC6, SLC1A2, | 0.865 | 0.855 | 0.876 | 0.908 | 0.850 | 0.965 | 0.942 | 0.868 | 1 |
| FAM20A, MMP8 | | | | | | | | | |
| OAS1, SIGLEC1, HERC6, SLC1A2, IL1R2, | 0.865 | 0.855 | 0.876 | 0.915 | 0.862 | 0.969 | 0.957 | 0.913 | 1.000 |
| OLAH, RETN | | | | | | | | | |
| OAS1, IFI44L, SIGLEC1, HERC6, SLC1A2, | 0.865 | 0.855 | 0.875 | 0.910 | 0.852 | 0.967 | 0.943 | 0.871 | 1 |
| FAM20A, RETN | | | | | | | | | |
| RSAD2, SIGLEC1, HERC6, SLC1A2, OLAH, | 0.865 | 0.855 | 0.875 | 0.910 | 0.853 | 0.968 | 0.952 | 0.907 | 0.997 |
| RETN, MMP8 | | | | | | | | | |
| IFIT1, SIGLEC1, ISG15, HERC6, SLC1A2, | 0.865 | 0.856 | 0.875 | 0.909 | 0.854 | 0.964 | 0.955 | 0.912 | 0.998 |
| IL1R2, RETN | | | | | | | | | |
| OAS1, SIGLEC1, ISG15, SLC1A2, FAM20A, | 0.865 | 0.855 | 0.876 | 0.904 | 0.840 | 0.967 | 0.950 | 0.880 | 1 |
| RETN, MMP8 | | | | | | | | | |
| RSAD2, SIGLEC1, ISG15, SLC1A2, FAM20A, | 0.865 | 0.855 | 0.875 | 0.908 | 0.848 | 0.968 | 0.953 | 0.885 | 1 |
| RETN, MMP8 | | | | | | | | | |
| RSAD2, SIGLEC1, ISG15, HERC6, SLC1A2, | 0.865 | 0.856 | 0.875 | 0.911 | 0.856 | 0.967 | 0.948 | 0.900 | 0.995 |
| OLAH, MMP8 | | | | | | | | | |
| RSAD2, SIGLEC1, SLC1A2, IL1R2, OLAH, | 0.865 | 0.856 | 0.875 | 0.914 | 0.859 | 0.968 | 0.968 | 0.935 | 1 |
| RETN, MMP8 | | | | | | | | | |
| OAS1, SIGLEC1, ISG15, HERC6, SLC1A2, | 0.865 | 0.855 | 0.875 | 0.913 | 0.856 | 0.969 | 0.950 | 0.900 | 1 |
| OLAH, RETN | | | | | | | | | |
| RSAD2, OAS1, SIGLEC1, ISG15, HERC6, | 0.865 | 0.855 | 0.875 | 0.912 | 0.856 | 0.967 | 0.951 | 0.904 | 0.998 |
| SLC1A2, OLAH | | | | | | | | | |
| IFI27, IFIT1, ISG15, SLC1A2, FAM20A, RETN, | 0.865 | 0.854 | 0.876 | 0.908 | 0.845 | 0.971 | 0.941 | 0.861 | 1 |
| MMP8 | | | | | | | | | |
| RSAD2, HERC6, SLC1A2, IL1R2, OLAH, | 0.865 | 0.855 | 0.875 | 0.913 | 0.858 | 0.967 | 0.927 | 0.842 | 1 |
| FAM20A, RETN | | | | | | | | | |
| IFI27, OAS1, ISG15, HERC6, SLC1A2, FAM20A, | 0.865 | 0.855 | 0.876 | 0.915 | 0.861 | 0.969 | 0.924 | 0.839 | 1 |
| MMP8 | | | | | | | | | |
| RSAD2, IFIT1, SIGLEC1, ISG15, SLC1A2, | 0.865 | 0.856 | 0.874 | 0.911 | 0.856 | 0.967 | 0.963 | 0.925 | 1 |
| IL1R2, OLAH | | | | | | | | | |
| IFIT1, IFI44L, SLC1A2, IL1R2, OLAH, FAM20A, | 0.865 | 0.855 | 0.875 | 0.913 | 0.858 | 0.968 | 0.926 | 0.846 | 1 |
| RETN | | | | | | | | | |
| IFIT1, SLC1A2, IL1R2, OLAH, FAM20A, RETN, | 0.865 | 0.855 | 0.875 | 0.913 | 0.859 | 0.967 | 0.938 | 0.864 | 1 |
| MMP8 | | | | | | | | | |
| IFI44L, SIGLEC1, ISG15, SLC1A2, OLAH, | 0.865 | 0.856 | 0.875 | 0.910 | 0.852 | 0.967 | 0.959 | 0.917 | 1.000 |
| RETN, MMP8 | | | | | | | | | |
| IFI44L, ISG15, HERC6, SLC1A2, OLAH, | 0.865 | 0.855 | 0.876 | 0.913 | 0.856 | 0.969 | 0.932 | 0.854 | 1 |
| FAM20A, RETN | | | | | | | | | |
| RSAD2, OAS1, SIGLEC1, ISG15, HERC6, | 0.865 | 0.856 | 0.875 | 0.909 | 0.855 | 0.963 | 0.966 | 0.929 | 1 |
| SLC1A2, IL1R2 | | | | | | | | | |
| HERC6, SLC1A2, IL1R2, OLAH, FAM20A, | 0.865 | 0.855 | 0.875 | 0.914 | 0.862 | 0.967 | 0.925 | 0.838 | 1 |
| RETN, MMP8 | | | | | | | | | |
| IFIT1, IFI44L, SIGLEC1, ISG15, SLC1A2, IL1R2, | 0.865 | 0.856 | 0.874 | 0.914 | 0.859 | 0.968 | 0.961 | 0.922 | 1 |
| OLAH | | | | | | | | | |
| OAS1, IFIT1, IFI44L, SIGLEC1, ISG15, SLC1A2, | 0.865 | 0.856 | 0.875 | 0.912 | 0.856 | 0.968 | 0.945 | 0.894 | 0.995 |
| OLAH | | | | | | | | | |
| OAS1, SIGLEC1, SLC1A2, IL1R2, OLAH, RETN, | 0.865 | 0.855 | 0.875 | 0.914 | 0.860 | 0.968 | 0.966 | 0.931 | 1 |
| MMP8 | | | | | | | | | |
| IFI44L, HERC6, SLC1A2, IL1R2, FAM20A, | 0.865 | 0.855 | 0.875 | 0.917 | 0.865 | 0.969 | 0.928 | 0.837 | 1 |
| RETN, MMP8 | | | | | | | | | |
| OAS1, ISG15, HERC6, SLC1A2, IL1R2, | 0.865 | 0.855 | 0.875 | 0.914 | 0.862 | 0.966 | 0.924 | 0.831 | 1 |
| FAM20A, MMP8 | | | | | | | | | |
| RSAD2, IFI44L, ISG15, SLC1A2, IL1R2, OLAH, | 0.865 | 0.855 | 0.875 | 0.912 | 0.856 | 0.968 | 0.929 | 0.852 | 1 |
| FAM20A | | | | | | | | | |
| RSAD2, OAS1, IFIT1, HERC6, IL1R2, FAM20A, | 0.865 | 0.855 | 0.875 | 0.905 | 0.842 | 0.967 | 0.937 | 0.856 | 1 |
| RETN | | | | | | | | | |
| IFIT1, IFI44L, ISG15, SLC1A2, OLAH, FAM20A, | 0.865 | 0.855 | 0.875 | 0.910 | 0.851 | 0.969 | 0.918 | 0.841 | 0.996 |
| RETN | | | | | | | | | |
| RSAD2, OAS1, IFI44L, SIGLEC1, HERC6, | 0.865 | 0.855 | 0.875 | 0.903 | 0.843 | 0.964 | 0.959 | 0.917 | 1 |
| IL1R2, OLAH | | | | | | | | | |
| OAS1, IFI44L, SIGLEC1, ISG15, HERC6, IL1R2, | 0.865 | 0.856 | 0.874 | 0.902 | 0.843 | 0.961 | 0.960 | 0.919 | 1 |
| MMP8 | | | | | | | | | |
| RSAD2, OAS1, IFIT1, IFI44L, HERC6, OLAH, | 0.865 | 0.855 | 0.875 | 0.903 | 0.841 | 0.966 | 0.933 | 0.863 | 1 |
| FAM20A | | | | | | | | | |
| RSAD2, OAS1, IFI44L, SIGLEC1, OLAH, | 0.865 | 0.855 | 0.875 | 0.896 | 0.824 | 0.969 | 0.958 | 0.911 | 1 |
| FAM20A, RETN | | | | | | | | | |
| RSAD2, IFIT1, IFI44L, SIGLEC1, HERC6, IL1R2, | 0.865 | 0.856 | 0.874 | 0.905 | 0.845 | 0.965 | 0.954 | 0.910 | 0.998 |
| OLAH | | | | | | | | | |
| IFIT1, ISG15, HERC6, OLAH, FAM20A, RETN, | 0.865 | 0.855 | 0.875 | 0.904 | 0.840 | 0.967 | 0.948 | 0.883 | 1 |
| MMP8 | | | | | | | | | |
| IFI27, IFIT1, ISG15, HERC6, SLC1A2, FAM20A, | 0.865 | 0.855 | 0.875 | 0.910 | 0.853 | 0.967 | 0.927 | 0.843 | 1 |
| MMP8 | | | | | | | | | |
| RSAD2, OAS1, IFIT1, ISG15, IL1R2, FAM20A, | 0.865 | 0.855 | 0.875 | 0.907 | 0.845 | 0.969 | 0.945 | 0.872 | 1 |
| MMP8 | | | | | | | | | |
| IFI27, IFIT1, SIGLEC1, ISG15, HERC6, SLC1A2, | 0.865 | 0.856 | 0.874 | 0.910 | 0.856 | 0.964 | 0.917 | 0.848 | 0.987 |
| MMP8 | | | | | | | | | |
| RSAD2, IFI44L, SIGLEC1, HERC6, IL1R2, | 0.865 | 0.856 | 0.874 | 0.902 | 0.842 | 0.962 | 0.962 | 0.922 | 1 |
| OLAH, MMP8 | | | | | | | | | |
| RSAD2, OAS1, IFI44L, SIGLEC1, ISG15, | 0.865 | 0.856 | 0.874 | 0.902 | 0.842 | 0.962 | 0.960 | 0.919 | 1 |
| HERC6, IL1R2 | | | | | | | | | |
| RSAD2, OAS1, IFIT1, SIGLEC1, HERC6, OLAH, | 0.865 | 0.855 | 0.875 | 0.902 | 0.838 | 0.966 | 0.940 | 0.888 | 0.993 |
| RETN | | | | | | | | | |
| RSAD2, OAS1, IFIT1, SIGLEC1, ISG15, HERC6, | 0.865 | 0.856 | 0.874 | 0.905 | 0.848 | 0.962 | 0.957 | 0.913 | 1 |
| IL1R2 | | | | | | | | | |
| IFIT1, SIGLEC1, ISG15, SLC1A2, OLAH, RETN, | 0.865 | 0.855 | 0.874 | 0.911 | 0.855 | 0.968 | 0.959 | 0.917 | 1 |
| MMP8 | | | | | | | | | |
| RSAD2, IFI44L, SIGLEC1, HERC6, SLC1A2, | 0.865 | 0.855 | 0.875 | 0.912 | 0.855 | 0.969 | 0.943 | 0.870 | 1 |
| FAM20A, RETN | | | | | | | | | |
| OAS1, IFIT1, SIGLEC1, ISG15, HERC6, | 0.865 | 0.855 | 0.874 | 0.910 | 0.858 | 0.963 | 0.959 | 0.918 | 1.000 |
| SLC1A2, IL1R2 | | | | | | | | | |
| RSAD2, IFI44L, SIGLEC1, HERC6, SLC1A2, | 0.865 | 0.855 | 0.875 | 0.909 | 0.852 | 0.966 | 0.937 | 0.860 | 1 |
| FAM20A, MMP8 | | | | | | | | | |
| IFIT1, IFI44L, SIGLEC1, HERC6, SLC1A2, | 0.865 | 0.855 | 0.874 | 0.913 | 0.859 | 0.967 | 0.953 | 0.909 | 0.997 |
| IL1R2, OLAH | | | | | | | | | |
| RSAD2, IFI44L, SIGLEC1, SLC1A2, IL1R2, | 0.865 | 0.856 | 0.874 | 0.912 | 0.858 | 0.966 | 0.967 | 0.933 | 1 |
| OLAH, MMP8 | | | | | | | | | |
| OAS1, IFIT1, IFI44L, HERC6, OLAH, FAM20A, | 0.865 | 0.855 | 0.874 | 0.903 | 0.840 | 0.967 | 0.935 | 0.865 | 1 |
| RETN | | | | | | | | | |
| RSAD2, IFI27, IFIT1, SIGLEC1, ISG15, HERC6, | 0.865 | 0.856 | 0.873 | 0.906 | 0.850 | 0.962 | 0.905 | 0.830 | 0.981 |
| MMP8 | | | | | | | | | |
| IFIT1, IFI44L, SIGLEC1, HERC6, IL1R2, OLAH, | 0.865 | 0.855 | 0.874 | 0.905 | 0.843 | 0.967 | 0.949 | 0.902 | 0.996 |
| RETN | | | | | | | | | |
| OAS1, IFIT1, IFI44L, HERC6, OLAH, FAM20A, | 0.865 | 0.855 | 0.875 | 0.902 | 0.839 | 0.965 | 0.938 | 0.870 | 1 |
| MMP8 | | | | | | | | | |
| OAS1, IFIT1, SIGLEC1, ISG15, HERC6, RETN, | 0.865 | 0.855 | 0.875 | 0.903 | 0.839 | 0.966 | 0.934 | 0.877 | 0.991 |
| MMP8 | | | | | | | | | |
| OAS1, ISG15, HERC6, SLC1A2, OLAH, | 0.865 | 0.854 | 0.875 | 0.911 | 0.854 | 0.968 | 0.934 | 0.855 | 1 |
| FAM20A, RETN | | | | | | | | | |
| RSAD2, OAS1, IFIT1, IFI44L, SIGLEC1, HERC6, | 0.865 | 0.855 | 0.874 | 0.900 | 0.833 | 0.967 | 0.925 | 0.865 | 0.985 |
| RETN | | | | | | | | | |
| OAS1, ISG15, SLC1A2, IL1R2, OLAH, FAM20A, | 0.865 | 0.855 | 0.874 | 0.915 | 0.862 | 0.968 | 0.926 | 0.842 | 1 |
| MMP8 | | | | | | | | | |
| RSAD2, OAS1, SIGLEC1, ISG15, HERC6, IL1R2, | 0.865 | 0.855 | 0.874 | 0.904 | 0.847 | 0.961 | 0.965 | 0.928 | 1 |
| MMP8 | | | | | | | | | |
| OAS1, IFIT1, ISG15, HERC6, OLAH, FAM20A, | 0.865 | 0.855 | 0.875 | 0.903 | 0.840 | 0.965 | 0.942 | 0.875 | 1 |
| RETN | | | | | | | | | |
| RSAD2, IFIT1, SIGLEC1, HERC6, IL1R2, OLAH, | 0.865 | 0.855 | 0.874 | 0.904 | 0.841 | 0.967 | 0.949 | 0.902 | 0.996 |
| RETN | | | | | | | | | |
| RSAD2, IFIT1, SIGLEC1, HERC6, IL1R2, OLAH, | 0.865 | 0.855 | 0.874 | 0.906 | 0.849 | 0.964 | 0.959 | 0.917 | 1 |
| MMP8 | | | | | | | | | |
| IFI44L, ISG15, HERC6, SLC1A2, IL1R2, | 0.865 | 0.855 | 0.875 | 0.913 | 0.859 | 0.967 | 0.928 | 0.841 | 1 |
| FAM20A, RETN | | | | | | | | | |
| RSAD2, OAS1, IFI44L, HERC6, SLC1A2, IL1R2, | 0.865 | 0.855 | 0.875 | 0.913 | 0.860 | 0.965 | 0.924 | 0.834 | 1 |
| FAM20A | | | | | | | | | |
| IFI44L, SIGLEC1, ISG15, HERC6, SLC1A2, | 0.865 | 0.855 | 0.874 | 0.911 | 0.856 | 0.966 | 0.955 | 0.912 | 0.998 |
| IL1R2, OLAH | | | | | | | | | |
| RSAD2, OAS1, SIGLEC1, HERC6, SLC1A2, | 0.865 | 0.855 | 0.875 | 0.913 | 0.856 | 0.970 | 0.952 | 0.903 | 1 |
| OLAH, RETN | | | | | | | | | |
| OAS1, IFI44L, HERC6, IL1R2, OLAH, FAM20A, | 0.865 | 0.855 | 0.875 | 0.905 | 0.844 | 0.967 | 0.939 | 0.867 | 1 |
| RETN | | | | | | | | | |
| OAS1, IFIT1, IFI44L, HERC6, IL1R2, FAM20A, | 0.865 | 0.855 | 0.874 | 0.906 | 0.844 | 0.968 | 0.936 | 0.855 | 1 |
| RETN | | | | | | | | | |
| RSAD2, OAS1, SIGLEC1, SLC1A2, IL1R2, | 0.865 | 0.855 | 0.874 | 0.913 | 0.857 | 0.968 | 0.967 | 0.929 | 1 |
| OLAH, RETN | | | | | | | | | |
| RSAD2, OAS1, HERC6, SLC1A2, IL1R2, | 0.865 | 0.855 | 0.874 | 0.914 | 0.862 | 0.966 | 0.925 | 0.832 | 1 |
| FAM20A, MMP8 | | | | | | | | | |
| OAS1, IFIT1, SIGLEC1, ISG15, HERC6, IL1R2, | 0.865 | 0.855 | 0.874 | 0.907 | 0.850 | 0.965 | 0.953 | 0.909 | 0.998 |
| OLAH | | | | | | | | | |
| RSAD2, IFI27, OAS1, IFIT1, SLC1A2, FAM20A, | 0.865 | 0.855 | 0.875 | 0.908 | 0.847 | 0.969 | 0.940 | 0.860 | 1 |
| RETN | | | | | | | | | |
| RSAD2, IFI44L, SIGLEC1, ISG15, HERC6, | 0.865 | 0.855 | 0.875 | 0.903 | 0.838 | 0.967 | 0.943 | 0.875 | 1 |
| FAM20A, MMP8 | | | | | | | | | |
| OAS1, IFI44L, SIGLEC1, ISG15, OLAH, | 0.865 | 0.854 | 0.875 | 0.896 | 0.824 | 0.968 | 0.961 | 0.915 | 1 |
| FAM20A, RETN | | | | | | | | | |
| IFIT1, SIGLEC1, ISG15, SLC1A2, IL1R2, OLAH, | 0.865 | 0.855 | 0.874 | 0.913 | 0.858 | 0.968 | 0.959 | 0.916 | 1 |
| RETN | | | | | | | | | |
| IFI44L, ISG15, SLC1A2, IL1R2, FAM20A, RETN, | 0.865 | 0.856 | 0.874 | 0.913 | 0.861 | 0.965 | 0.927 | 0.836 | 1 |
| MMP8 | | | | | | | | | |
| RSAD2, IFI27, IFIT1, IFI44L, SIGLEC1, HERC6, | 0.865 | 0.856 | 0.873 | 0.906 | 0.850 | 0.962 | 0.904 | 0.829 | 0.980 |
| MMP8 | | | | | | | | | |
| RSAD2, IFI27, IFIT1, SIGLEC1, HERC6, | 0.865 | 0.856 | 0.873 | 0.909 | 0.854 | 0.964 | 0.916 | 0.846 | 0.986 |
| SLC1A2, MMP8 | | | | | | | | | |
| RSAD2, OAS1, HERC6, SLC1A2, OLAH, | 0.865 | 0.855 | 0.874 | 0.912 | 0.857 | 0.967 | 0.932 | 0.852 | 1 |
| FAM20A, MMP8 | | | | | | | | | |
| RSAD2, IFI27, OAS1, IFI44L, HERC6, SLC1A2, | 0.865 | 0.854 | 0.876 | 0.914 | 0.857 | 0.971 | 0.926 | 0.843 | 1 |
| FAM20A | | | | | | | | | |
| RSAD2, IFIT1, ISG15, HERC6, SLC1A2, IL1R2, | 0.865 | 0.855 | 0.874 | 0.912 | 0.858 | 0.965 | 0.928 | 0.842 | 1 |
| FAM20A | | | | | | | | | |
| OAS1, IFIT1, SIGLEC1, ISG15, HERC6, | 0.864 | 0.855 | 0.874 | 0.912 | 0.856 | 0.968 | 0.929 | 0.869 | 0.990 |
| SLC1A2, RETN | | | | | | | | | |
| SIGLEC1, ISG15, HERC6, IL1R2, OLAH, RETN, | 0.864 | 0.855 | 0.874 | 0.901 | 0.836 | 0.965 | 0.968 | 0.935 | 1 |
| MMP8 | | | | | | | | | |
| RSAD2, OAS1, IFIT1, IFI44L, SIGLEC1, | 0.864 | 0.855 | 0.874 | 0.912 | 0.856 | 0.968 | 0.952 | 0.904 | 1 |
| SLC1A2, OLAH | | | | | | | | | |
| OAS1, SIGLEC1, ISG15, HERC6, SLC1A2, | 0.864 | 0.855 | 0.874 | 0.910 | 0.858 | 0.963 | 0.966 | 0.930 | 1 |
| IL1R2, MMP8 | | | | | | | | | |
| IFIT1, ISG15, HERC6, SLC1A2, IL1R2, | 0.864 | 0.854 | 0.875 | 0.911 | 0.855 | 0.966 | 0.930 | 0.845 | 1 |
| FAM20A, RETN | | | | | | | | | |
| RSAD2, SIGLEC1, ISG15, HERC6, IL1R2, | 0.864 | 0.855 | 0.874 | 0.905 | 0.847 | 0.963 | 0.960 | 0.919 | 1 |
| OLAH, MMP8 | | | | | | | | | |
| RSAD2, IFIT1, SIGLEC1, HERC6, OLAH, RETN, | 0.864 | 0.854 | 0.874 | 0.901 | 0.836 | 0.967 | 0.957 | 0.915 | 0.998 |
| MMP8 | | | | | | | | | |
| RSAD2, OAS1, IFIT1, SIGLEC1, HERC6, | 0.864 | 0.855 | 0.874 | 0.908 | 0.853 | 0.962 | 0.965 | 0.928 | 1 |
| SLC1A2, IL1R2 | | | | | | | | | |
| RSAD2, IFI27, IFIT1, SLC1A2, FAM20A, RETN, | 0.864 | 0.854 | 0.875 | 0.908 | 0.845 | 0.971 | 0.941 | 0.861 | 1 |
| MMP8 | | | | | | | | | |
| RSAD2, ISG15, HERC6, SLC1A2, IL1R2, | 0.864 | 0.854 | 0.875 | 0.911 | 0.857 | 0.966 | 0.929 | 0.841 | 1 |
| FAM20A, RETN | | | | | | | | | |
| RSAD2, IFIT1, SIGLEC1, HERC6, SLC1A2, | 0.864 | 0.855 | 0.874 | 0.909 | 0.853 | 0.965 | 0.951 | 0.906 | 0.997 |
| IL1R2, RETN | | | | | | | | | |
| IFI44L, SIGLEC1, ISG15, HERC6, SLC1A2, | 0.864 | 0.854 | 0.874 | 0.902 | 0.840 | 0.965 | 0.952 | 0.879 | 1 |
| FAM20A, MMP8 | | | | | | | | | |
| OAS1, IFIT1, HERC6, SLC1A2, OLAH, | 0.864 | 0.854 | 0.875 | 0.909 | 0.853 | 0.965 | 0.932 | 0.853 | 1 |
| FAM20A, MMP8 | | | | | | | | | |
| OAS1, IFI44L, SIGLEC1, HERC6, SLC1A2, | 0.864 | 0.854 | 0.874 | 0.911 | 0.855 | 0.967 | 0.954 | 0.910 | 0.999 |
| OLAH, MMP8 | | | | | | | | | |
| RSAD2, OAS1, IFI44L, SLC1A2, IL1R2, OLAH, | 0.864 | 0.854 | 0.875 | 0.913 | 0.857 | 0.969 | 0.930 | 0.852 | 1 |
| FAM20A | | | | | | | | | |
| OAS1, SIGLEC1, ISG15, SLC1A2, OLAH, RETN, | 0.864 | 0.854 | 0.874 | 0.911 | 0.854 | 0.968 | 0.960 | 0.919 | 1 |
| MMP8 | | | | | | | | | |
| OAS1, IFIT1, SIGLEC1, ISG15, SLC1A2, OLAH, | 0.864 | 0.854 | 0.874 | 0.914 | 0.858 | 0.969 | 0.943 | 0.892 | 0.995 |
| RETN | | | | | | | | | |
| RSAD2, SIGLEC1, ISG15, SLC1A2, IL1R2, | 0.864 | 0.855 | 0.874 | 0.914 | 0.859 | 0.969 | 0.960 | 0.919 | 1 |
| OLAH, RETN | | | | | | | | | |
| RSAD2, OAS1, SIGLEC1, ISG15, SLC1A2, | 0.864 | 0.855 | 0.874 | 0.911 | 0.856 | 0.967 | 0.953 | 0.908 | 0.998 |
| OLAH, MMP8 | | | | | | | | | |
| OAS1, IFI44L, SIGLEC1, ISG15, HERC6, | 0.864 | 0.854 | 0.874 | 0.909 | 0.852 | 0.966 | 0.942 | 0.868 | 1 |
| SLC1A2, FAM20A | | | | | | | | | |
| RSAD2, OAS1, IFIT1, SIGLEC1, ISG15, | 0.864 | 0.855 | 0.874 | 0.911 | 0.855 | 0.968 | 0.949 | 0.900 | 0.998 |
| SLC1A2, OLAH | | | | | | | | | |
| RSAD2, OAS1, IFIT1, SLC1A2, IL1R2, FAM20A, | 0.864 | 0.855 | 0.873 | 0.914 | 0.860 | 0.967 | 0.936 | 0.855 | 1 |
| MMP8 | | | | | | | | | |
| OAS1, IFI44L, ISG15, IL1R2, OLAH, FAM20A, | 0.864 | 0.854 | 0.874 | 0.905 | 0.843 | 0.968 | 0.943 | 0.872 | 1 |
| MMP8 | | | | | | | | | |
| RSAD2, OAS1, SIGLEC1, ISG15, SLC1A2, | 0.864 | 0.855 | 0.874 | 0.912 | 0.856 | 0.967 | 0.966 | 0.929 | 1 |
| IL1R2, OLAH | | | | | | | | | |
| IFI44L, SIGLEC1, HERC6, SLC1A2, IL1R2, | 0.864 | 0.854 | 0.874 | 0.914 | 0.859 | 0.969 | 0.959 | 0.917 | 1 |
| OLAH, RETN | | | | | | | | | |
| RSAD2, OAS1, SIGLEC1, ISG15, HERC6, IL1R2, | 0.864 | 0.854 | 0.874 | 0.907 | 0.849 | 0.964 | 0.963 | 0.924 | 1 |
| OLAH | | | | | | | | | |
| RSAD2, IFI44L, SIGLEC1, ISG15, HERC6, | 0.864 | 0.855 | 0.873 | 0.902 | 0.842 | 0.962 | 0.960 | 0.919 | 1 |
| IL1R2, MMP8 | | | | | | | | | |
| IFI44L, SIGLEC1, HERC6, SLC1A2, IL1R2, | 0.864 | 0.855 | 0.873 | 0.910 | 0.854 | 0.965 | 0.966 | 0.931 | 1 |
| RETN, MMP8 | | | | | | | | | |
| RSAD2, OAS1, ISG15, HERC6, SLC1A2, OLAH, | 0.864 | 0.854 | 0.874 | 0.911 | 0.855 | 0.967 | 0.929 | 0.849 | 1 |
| FAM20A | | | | | | | | | |
| IFIT1, HERC6, SLC1A2, OLAH, FAM20A, | 0.864 | 0.854 | 0.875 | 0.908 | 0.850 | 0.967 | 0.933 | 0.854 | 1 |
| RETN, MMP8 | | | | | | | | | |
| OAS1, SIGLEC1, ISG15, HERC6, IL1R2, OLAH, | 0.864 | 0.855 | 0.874 | 0.905 | 0.847 | 0.962 | 0.963 | 0.924 | 1 |
| MMP8 | | | | | | | | | |
| OAS1, IFIT1, IL1R2, OLAH, FAM20A, RETN, | 0.864 | 0.854 | 0.874 | 0.905 | 0.841 | 0.968 | 0.946 | 0.875 | 1 |
| MMP8 | | | | | | | | | |
| RSAD2, IFI27, ISG15, HERC6, SLC1A2, | 0.864 | 0.854 | 0.875 | 0.913 | 0.858 | 0.969 | 0.927 | 0.843 | 1 |
| FAM20A, MMP8 | | | | | | | | | |
| RSAD2, OAS1, IFI44L, SIGLEC1, FAM20A, | 0.864 | 0.854 | 0.875 | 0.903 | 0.835 | 0.970 | 0.953 | 0.889 | 1 |
| RETN, MMP8 | | | | | | | | | |
| SIGLEC1, ISG15, HERC6, SLC1A2, OLAH, | 0.864 | 0.854 | 0.874 | 0.910 | 0.850 | 0.969 | 0.958 | 0.916 | 0.999 |
| RETN, MMP8 | | | | | | | | | |
| OAS1, IFIT1, ISG15, SLC1A2, IL1R2, OLAH, | 0.864 | 0.854 | 0.874 | 0.911 | 0.853 | 0.968 | 0.935 | 0.856 | 1 |
| FAM20A | | | | | | | | | |
| OAS1, IFI44L, HERC6, SLC1A2, OLAH, | 0.864 | 0.853 | 0.875 | 0.912 | 0.857 | 0.968 | 0.924 | 0.843 | 1 |
| FAM20A, RETN | | | | | | | | | |
| RSAD2, IFI27, OAS1, IFI44L, ISG15, FAM20A, | 0.864 | 0.853 | 0.875 | 0.901 | 0.834 | 0.967 | 0.934 | 0.855 | 1 |
| MMP8 | | | | | | | | | |
| RSAD2, IFIT1, IFI44L, SLC1A2, IL1R2, OLAH, | 0.864 | 0.855 | 0.873 | 0.913 | 0.858 | 0.969 | 0.928 | 0.850 | 1 |
| FAM20A | | | | | | | | | |
| OAS1, IFI44L, SIGLEC1, ISG15, HERC6, | 0.864 | 0.854 | 0.874 | 0.912 | 0.857 | 0.968 | 0.951 | 0.904 | 0.998 |
| SLC1A2, OLAH | | | | | | | | | |
| OAS1, IFIT1, HERC6, SLC1A2, IL1R2, OLAH, | 0.864 | 0.854 | 0.874 | 0.912 | 0.857 | 0.967 | 0.927 | 0.843 | 1 |
| FAM20A | | | | | | | | | |
| OAS1, IFI44L, ISG15, SLC1A2, IL1R2, OLAH, | 0.864 | 0.854 | 0.874 | 0.913 | 0.857 | 0.969 | 0.929 | 0.851 | 1 |
| FAM20A | | | | | | | | | |
| OAS1, IFIT1, IFI44L, HERC6, SLC1A2, IL1R2, | 0.864 | 0.854 | 0.874 | 0.913 | 0.861 | 0.965 | 0.924 | 0.832 | 1 |
| FAM20A | | | | | | | | | |
| IFI27, OAS1, ISG15, SLC1A2, FAM20A, RETN, | 0.864 | 0.853 | 0.875 | 0.911 | 0.850 | 0.972 | 0.942 | 0.863 | 1 |
| MMP8 | | | | | | | | | |
| RSAD2, IFI44L, HERC6, IL1R2, OLAH, | 0.864 | 0.854 | 0.874 | 0.906 | 0.846 | 0.967 | 0.938 | 0.865 | 1 |
| FAM20A, RETN | | | | | | | | | |
| IFIT1, IFI44L, ISG15, IL1R2, OLAH, FAM20A, | 0.864 | 0.855 | 0.873 | 0.906 | 0.845 | 0.967 | 0.950 | 0.888 | 1 |
| MMP8 | | | | | | | | | |
| OAS1, IFI44L, SIGLEC1, SLC1A2, FAM20A, | 0.864 | 0.854 | 0.874 | 0.905 | 0.843 | 0.968 | 0.950 | 0.880 | 1 |
| RETN, MMP8 | | | | | | | | | |
| RSAD2, OAS1, IFI44L, HERC6, IL1R2, | 0.864 | 0.854 | 0.874 | 0.908 | 0.847 | 0.969 | 0.936 | 0.858 | 1 |
| FAM20A, RETN | | | | | | | | | |
| RSAD2, ISG15, SLC1A2, IL1R2, OLAH, | 0.864 | 0.855 | 0.873 | 0.914 | 0.862 | 0.967 | 0.929 | 0.844 | 1 |
| FAM20A, MMP8 | | | | | | | | | |
| RSAD2, OAS1, SIGLEC1, SLC1A2, OLAH, | 0.864 | 0.854 | 0.874 | 0.912 | 0.854 | 0.969 | 0.962 | 0.921 | 1 |
| RETN, MMP8 | | | | | | | | | |
| IFIT1, IFI44L, ISG15, SLC1A2, IL1R2, FAM20A, | 0.864 | 0.855 | 0.873 | 0.912 | 0.859 | 0.964 | 0.930 | 0.843 | 1 |
| MMP8 | | | | | | | | | |
| IFIT1, IFI44L, SIGLEC1, HERC6, SLC1A2, | 0.864 | 0.855 | 0.873 | 0.908 | 0.855 | 0.962 | 0.964 | 0.927 | 1 |
| IL1R2, MMP8 | | | | | | | | | |
| RSAD2, OAS1, IFIT1, ISG15, IL1R2, OLAH, | 0.864 | 0.854 | 0.874 | 0.901 | 0.835 | 0.968 | 0.945 | 0.873 | 1 |
| FAM20A | | | | | | | | | |
| IFI27, OAS1, IFIT1, HERC6, SLC1A2, FAM20A, | 0.864 | 0.854 | 0.874 | 0.914 | 0.859 | 0.969 | 0.925 | 0.840 | 1 |
| MMP8 | | | | | | | | | |
| RSAD2, IFI44L, SIGLEC1, ISG15, HERC6, | 0.864 | 0.854 | 0.874 | 0.901 | 0.838 | 0.964 | 0.958 | 0.916 | 1.000 |
| IL1R2, RETN | | | | | | | | | |
| IFI27, IFIT1, IFI44L, SIGLEC1, HERC6, SLC1A2, | 0.864 | 0.855 | 0.873 | 0.909 | 0.854 | 0.963 | 0.920 | 0.850 | 0.989 |
| MMP8 | | | | | | | | | |
| IFIT1, ISG15, HERC6, SLC1A2, IL1R2, | 0.864 | 0.854 | 0.874 | 0.915 | 0.863 | 0.967 | 0.929 | 0.842 | 1 |
| FAM20A, MMP8 | | | | | | | | | |
| RSAD2, IFIT1, SIGLEC1, SLC1A2, OLAH, | 0.864 | 0.854 | 0.874 | 0.913 | 0.857 | 0.969 | 0.960 | 0.919 | 1 |
| RETN, MMP8 | | | | | | | | | |
| IFIT1, IFI44L, SIGLEC1, HERC6, SLC1A2, | 0.864 | 0.855 | 0.873 | 0.910 | 0.855 | 0.965 | 0.950 | 0.904 | 0.996 |
| IL1R2, RETN | | | | | | | | | |
| RSAD2, OAS1, ISG15, SLC1A2, IL1R2, OLAH, | 0.864 | 0.854 | 0.874 | 0.911 | 0.855 | 0.968 | 0.933 | 0.850 | 1 |
| FAM20A | | | | | | | | | |
| RSAD2, IFIT1, SIGLEC1, ISG15, HERC6, IL1R2, | 0.864 | 0.855 | 0.873 | 0.904 | 0.847 | 0.962 | 0.957 | 0.913 | 1 |
| MMP8 | | | | | | | | | |
| RSAD2, IFIT1, SIGLEC1, SLC1A2, IL1R2, | 0.864 | 0.855 | 0.873 | 0.913 | 0.857 | 0.969 | 0.961 | 0.920 | 1 |
| OLAH, RETN | | | | | | | | | |
| RSAD2, OAS1, IFIT1, SIGLEC1, SLC1A2, | 0.864 | 0.854 | 0.874 | 0.912 | 0.856 | 0.968 | 0.958 | 0.910 | 1 |
| OLAH, RETN | | | | | | | | | |
| OAS1, IFIT1, IFI44L, ISG15, HERC6, FAM20A, | 0.864 | 0.854 | 0.874 | 0.906 | 0.844 | 0.969 | 0.928 | 0.848 | 1 |
| RETN | | | | | | | | | |
| OAS1, HERC6, SLC1A2, IL1R2, OLAH, | 0.864 | 0.853 | 0.874 | 0.912 | 0.856 | 0.967 | 0.927 | 0.842 | 1 |
| FAM20A, RETN | | | | | | | | | |
| RSAD2, HERC6, SLC1A2, OLAH, FAM20A, | 0.864 | 0.854 | 0.874 | 0.910 | 0.852 | 0.967 | 0.934 | 0.854 | 1 |
| RETN, MMP8 | | | | | | | | | |
| IFI44L, ISG15, HERC6, OLAH, FAM20A, RETN, | 0.864 | 0.853 | 0.874 | 0.903 | 0.836 | 0.969 | 0.950 | 0.886 | 1 |
| MMP8 | | | | | | | | | |
| OAS1, IFIT1, IFI44L, SLC1A2, IL1R2, OLAH, | 0.864 | 0.854 | 0.874 | 0.914 | 0.859 | 0.969 | 0.927 | 0.846 | 1 |
| FAM20A | | | | | | | | | |
| RSAD2, IFIT1, SIGLEC1, HERC6, SLC1A2, | 0.864 | 0.855 | 0.873 | 0.908 | 0.854 | 0.963 | 0.964 | 0.927 | 1 |
| IL1R2, MMP8 | | | | | | | | | |
| OAS1, ISG15, HERC6, OLAH, FAM20A, RETN, | 0.864 | 0.854 | 0.874 | 0.904 | 0.841 | 0.967 | 0.948 | 0.883 | 1 |
| MMP8 | | | | | | | | | |
| ISG15, HERC6, SLC1A2, OLAH, FAM20A, | 0.864 | 0.854 | 0.874 | 0.911 | 0.854 | 0.968 | 0.935 | 0.856 | 1 |
| RETN, MMP8 | | | | | | | | | |
| RSAD2, OAS1, IFI44L, ISG15, IL1R2, OLAH, | 0.864 | 0.854 | 0.874 | 0.901 | 0.836 | 0.966 | 0.948 | 0.882 | 1 |
| FAM20A | | | | | | | | | |
| RSAD2, IFI27, OAS1, IFIT1, IFI44L, ISG15, | 0.864 | 0.853 | 0.874 | 0.902 | 0.835 | 0.969 | 0.940 | 0.864 | 1 |
| FAM20A | | | | | | | | | |
| OAS1, IFIT1, IFI44L, ISG15, HERC6, OLAH, | 0.864 | 0.853 | 0.874 | 0.902 | 0.841 | 0.964 | 0.933 | 0.861 | 1 |
| FAM20A | | | | | | | | | |
| RSAD2, ISG15, HERC6, OLAH, FAM20A, | 0.864 | 0.854 | 0.874 | 0.905 | 0.843 | 0.967 | 0.947 | 0.882 | 1 |
| RETN, MMP8 | | | | | | | | | |
| OAS1, SIGLEC1, ISG15, HERC6, IL1R2, OLAH, | 0.864 | 0.854 | 0.874 | 0.905 | 0.844 | 0.966 | 0.958 | 0.915 | 1 |
| RETN | | | | | | | | | |
| RSAD2, SIGLEC1, ISG15, HERC6, SLC1A2, | 0.864 | 0.854 | 0.873 | 0.910 | 0.854 | 0.965 | 0.958 | 0.916 | 1.000 |
| IL1R2, RETN | | | | | | | | | |
| OAS1, IFI44L, SIGLEC1, ISG15, SLC1A2, | 0.864 | 0.854 | 0.873 | 0.912 | 0.856 | 0.968 | 0.952 | 0.907 | 0.998 |
| OLAH, MMP8 | | | | | | | | | |
| OAS1, IFI44L, SIGLEC1, HERC6, SLC1A2, | 0.864 | 0.854 | 0.873 | 0.911 | 0.856 | 0.966 | 0.952 | 0.906 | 0.998 |
| IL1R2, RETN | | | | | | | | | |
| RSAD2, OAS1, SIGLEC1, HERC6, SLC1A2, | 0.864 | 0.854 | 0.873 | 0.911 | 0.858 | 0.963 | 0.968 | 0.933 | 1 |
| IL1R2, MMP8 | | | | | | | | | |
| IFI44L, SIGLEC1, HERC6, SLC1A2, OLAH, | 0.864 | 0.854 | 0.874 | 0.910 | 0.851 | 0.968 | 0.962 | 0.923 | 1 |
| RETN, MMP8 | | | | | | | | | |
| OAS1, IFI44L, SIGLEC1, SLC1A2, IL1R2, | 0.864 | 0.854 | 0.873 | 0.912 | 0.858 | 0.966 | 0.967 | 0.933 | 1 |
| OLAH, MMP8 | | | | | | | | | |
| OAS1, HERC6, SLC1A2, OLAH, FAM20A, | 0.864 | 0.854 | 0.874 | 0.910 | 0.853 | 0.967 | 0.933 | 0.852 | 1 |
| RETN, MMP8 | | | | | | | | | |
| RSAD2, OAS1, IFI44L, SIGLEC1, SLC1A2, | 0.864 | 0.854 | 0.874 | 0.911 | 0.855 | 0.967 | 0.957 | 0.914 | 0.999 |
| OLAH, MMP8 | | | | | | | | | |
| RSAD2, OAS1, SIGLEC1, HERC6, IL1R2, | 0.864 | 0.854 | 0.874 | 0.902 | 0.838 | 0.965 | 0.959 | 0.916 | 1 |
| OLAH, RETN | | | | | | | | | |
| RSAD2, OAS1, IFI44L, SIGLEC1, HERC6, | 0.864 | 0.854 | 0.874 | 0.899 | 0.833 | 0.965 | 0.943 | 0.873 | 1 |
| FAM20A, MMP8 | | | | | | | | | |
| RSAD2, IFIT1, SIGLEC1, ISG15, HERC6, IL1R2, | 0.864 | 0.854 | 0.873 | 0.908 | 0.850 | 0.966 | 0.951 | 0.905 | 0.997 |
| OLAH | | | | | | | | | |
| OAS1, SIGLEC1, ISG15, SLC1A2, IL1R2, OLAH, | 0.864 | 0.854 | 0.873 | 0.912 | 0.857 | 0.968 | 0.960 | 0.919 | 1 |
| RETN | | | | | | | | | |
| RSAD2, IFIT1, HERC6, SLC1A2, OLAH, | 0.864 | 0.854 | 0.874 | 0.909 | 0.853 | 0.965 | 0.929 | 0.849 | 1 |
| FAM20A, MMP8 | | | | | | | | | |
| RSAD2, IFI44L, SIGLEC1, SLC1A2, FAM20A, | 0.864 | 0.854 | 0.874 | 0.910 | 0.849 | 0.970 | 0.950 | 0.883 | 1 |
| RETN, MMP8 | | | | | | | | | |
| RSAD2, IFIT1, HERC6, SLC1A2, IL1R2, OLAH, | 0.864 | 0.854 | 0.874 | 0.913 | 0.859 | 0.967 | 0.928 | 0.844 | 1 |
| FAM20A | | | | | | | | | |
| RSAD2, OAS1, SIGLEC1, ISG15, SLC1A2, | 0.864 | 0.853 | 0.874 | 0.902 | 0.840 | 0.965 | 0.945 | 0.870 | 1 |
| FAM20A, MMP8 | | | | | | | | | |
| RSAD2, ISG15, HERC6, SLC1A2, IL1R2, | 0.864 | 0.854 | 0.873 | 0.913 | 0.862 | 0.965 | 0.922 | 0.826 | 1 |
| FAM20A, MMP8 | | | | | | | | | |
| RSAD2, IFI44L, SIGLEC1, HERC6, SLC1A2, | 0.864 | 0.854 | 0.874 | 0.910 | 0.855 | 0.966 | 0.958 | 0.916 | 0.999 |
| IL1R2, OLAH | | | | | | | | | |
| OAS1, IFIT1, IFI44L, SIGLEC1, HERC6, | 0.864 | 0.854 | 0.873 | 0.910 | 0.857 | 0.963 | 0.959 | 0.918 | 1.000 |
| SLC1A2, IL1R2 | | | | | | | | | |
| RSAD2, IFIT1, IFI44L, ISG15, HERC6, OLAH, | 0.864 | 0.853 | 0.874 | 0.905 | 0.844 | 0.965 | 0.932 | 0.861 | 1 |
| FAM20A | | | | | | | | | |
| RSAD2, OAS1, IFI44L, SIGLEC1, HERC6, | 0.864 | 0.854 | 0.874 | 0.912 | 0.856 | 0.968 | 0.951 | 0.904 | 0.998 |
| SLC1A2, OLAH | | | | | | | | | |
| OAS1, IFIT1, ISG15, HERC6, SLC1A2, OLAH, | 0.864 | 0.853 | 0.874 | 0.911 | 0.855 | 0.967 | 0.930 | 0.851 | 1 |
| FAM20A | | | | | | | | | |
| RSAD2, IFIT1, SIGLEC1, ISG15, HERC6, OLAH, | 0.864 | 0.854 | 0.873 | 0.901 | 0.837 | 0.966 | 0.948 | 0.900 | 0.995 |
| RETN | | | | | | | | | |
| RSAD2, IFIT1, IFI44L, HERC6, SLC1A2, IL1R2, | 0.864 | 0.854 | 0.873 | 0.914 | 0.862 | 0.965 | 0.924 | 0.833 | 1 |
| FAM20A | | | | | | | | | |
| OAS1, IFIT1, IFI44L, SIGLEC1, IL1R2, OLAH, | 0.864 | 0.854 | 0.873 | 0.903 | 0.843 | 0.963 | 0.958 | 0.916 | 1.000 |
| MMP8 | | | | | | | | | |
| OAS1, IFIT1, SIGLEC1, IL1R2, OLAH, RETN, | 0.864 | 0.854 | 0.873 | 0.902 | 0.840 | 0.964 | 0.962 | 0.924 | 1 |
| MMP8 | | | | | | | | | |
| RSAD2, IFI44L, SLC1A2, IL1R2, OLAH, | 0.864 | 0.853 | 0.874 | 0.911 | 0.855 | 0.967 | 0.926 | 0.845 | 1 |
| FAM20A, RETN | | | | | | | | | |
| IFI27, OAS1, IFIT1, IFI44L, HERC6, SLC1A2, | 0.864 | 0.853 | 0.874 | 0.912 | 0.856 | 0.968 | 0.928 | 0.844 | 1 |
| FAM20A | | | | | | | | | |
| OAS1, IFIT1, IFI44L, ISG15, SLC1A2, OLAH, | 0.864 | 0.854 | 0.874 | 0.911 | 0.852 | 0.969 | 0.922 | 0.844 | 1.000 |
| FAM20A | | | | | | | | | |
| RSAD2, IFI27, OAS1, IFIT1, IFI44L, FAM20A, | 0.864 | 0.853 | 0.874 | 0.900 | 0.834 | 0.967 | 0.933 | 0.853 | 1 |
| MMP8 | | | | | | | | | |
| RSAD2, IFI44L, HERC6, SLC1A2, OLAH, | 0.864 | 0.853 | 0.874 | 0.914 | 0.859 | 0.969 | 0.924 | 0.843 | 1 |
| FAM20A, RETN | | | | | | | | | |
| SIGLEC1, ISG15, HERC6, SLC1A2, IL1R2, | 0.864 | 0.854 | 0.874 | 0.915 | 0.860 | 0.969 | 0.959 | 0.917 | 1 |
| OLAH, RETN | | | | | | | | | |
| RSAD2, OAS1, IFIT1, IFI44L, IL1R2, OLAH, | 0.864 | 0.854 | 0.873 | 0.901 | 0.835 | 0.966 | 0.946 | 0.878 | 1 |
| FAM20A | | | | | | | | | |
| OAS1, IFIT1, IFI44L, SIGLEC1, HERC6, | 0.864 | 0.854 | 0.873 | 0.907 | 0.849 | 0.966 | 0.925 | 0.864 | 0.986 |
| SLC1A2, RETN | | | | | | | | | |
| OAS1, IFI44L, SIGLEC1, ISG15, HERC6, IL1R2, | 0.864 | 0.854 | 0.873 | 0.904 | 0.845 | 0.964 | 0.960 | 0.919 | 1 |
| OLAH | | | | | | | | | |
| OAS1, IFI44L, SIGLEC1, HERC6, SLC1A2, | 0.864 | 0.854 | 0.874 | 0.907 | 0.850 | 0.964 | 0.942 | 0.867 | 1 |
| FAM20A, MMP8 | | | | | | | | | |
| IFIT1, SIGLEC1, ISG15, HERC6, SLC1A2, | 0.864 | 0.855 | 0.872 | 0.910 | 0.856 | 0.963 | 0.965 | 0.929 | 1 |
| IL1R2, MMP8 | | | | | | | | | |
| RSAD2, IFIT1, SIGLEC1, ISG15, HERC6, | 0.864 | 0.855 | 0.872 | 0.908 | 0.854 | 0.963 | 0.963 | 0.925 | 1 |
| SLC1A2, IL1R2 | | | | | | | | | |
| RSAD2, IFI44L, SIGLEC1, HERC6, SLC1A2, | 0.864 | 0.854 | 0.874 | 0.914 | 0.859 | 0.968 | 0.949 | 0.902 | 0.996 |
| OLAH, MMP8 | | | | | | | | | |
| OAS1, IFIT1, IFI44L, SIGLEC1, IL1R2, RETN, | 0.864 | 0.854 | 0.873 | 0.901 | 0.838 | 0.965 | 0.961 | 0.922 | 1 |
| MMP8 | | | | | | | | | |
| OAS1, HERC6, SLC1A2, IL1R2, FAM20A, | 0.864 | 0.854 | 0.873 | 0.914 | 0.861 | 0.968 | 0.928 | 0.839 | 1 |
| RETN, MMP8 | | | | | | | | | |
| OAS1, ISG15, HERC6, SLC1A2, OLAH, | 0.863 | 0.853 | 0.874 | 0.912 | 0.857 | 0.967 | 0.933 | 0.853 | 1 |
| FAM20A, MMP8 | | | | | | | | | |
| IFIT1, HERC6, SLC1A2, IL1R2, OLAH, | 0.863 | 0.853 | 0.874 | 0.912 | 0.857 | 0.967 | 0.927 | 0.843 | 1 |
| FAM20A, RETN | | | | | | | | | |
| OAS1, IFI44L, SIGLEC1, HERC6, SLC1A2, | 0.863 | 0.854 | 0.873 | 0.909 | 0.856 | 0.962 | 0.963 | 0.924 | 1 |
| IL1R2, MMP8 | | | | | | | | | |
| OAS1, IFIT1, HERC6, OLAH, FAM20A, RETN, | 0.863 | 0.853 | 0.873 | 0.901 | 0.837 | 0.966 | 0.950 | 0.886 | 1 |
| MMP8 | | | | | | | | | |
| IFIT1, IFI44L, HERC6, SLC1A2, IL1R2, | 0.863 | 0.854 | 0.873 | 0.915 | 0.863 | 0.966 | 0.923 | 0.834 | 1 |
| FAM20A, RETN | | | | | | | | | |
| IFIT1, IFI44L, SLC1A2, IL1R2, FAM20A, RETN, | 0.863 | 0.854 | 0.873 | 0.912 | 0.859 | 0.966 | 0.932 | 0.848 | 1 |
| MMP8 | | | | | | | | | |
| RSAD2, ISG15, HERC6, SLC1A2, OLAH, | 0.863 | 0.854 | 0.873 | 0.912 | 0.857 | 0.967 | 0.933 | 0.853 | 1 |
| FAM20A, MMP8 | | | | | | | | | |
| RSAD2, IFI44L, HERC6, IL1R2, FAM20A, | 0.863 | 0.853 | 0.874 | 0.910 | 0.849 | 0.970 | 0.936 | 0.853 | 1 |
| RETN, MMP8 | | | | | | | | | |
| RSAD2, SIGLEC1, ISG15, HERC6, SLC1A2, | 0.863 | 0.854 | 0.872 | 0.907 | 0.853 | 0.962 | 0.963 | 0.925 | 1 |
| IL1R2, MMP8 | | | | | | | | | |
| RSAD2, IFIT1, IFI44L, HERC6, OLAH, | 0.863 | 0.853 | 0.873 | 0.903 | 0.841 | 0.965 | 0.936 | 0.867 | 1 |
| FAM20A, MMP8 | | | | | | | | | |
| IFI27, OAS1, IFI44L, ISG15, HERC6, SLC1A2, | 0.863 | 0.853 | 0.874 | 0.910 | 0.853 | 0.968 | 0.932 | 0.849 | 1 |
| FAM20A | | | | | | | | | |
| RSAD2, IFI27, OAS1, SLC1A2, FAM20A, RETN, | 0.863 | 0.853 | 0.874 | 0.911 | 0.850 | 0.972 | 0.941 | 0.861 | 1 |
| MMP8 | | | | | | | | | |
| IFI44L, SIGLEC1, ISG15, HERC6, SLC1A2, | 0.863 | 0.853 | 0.873 | 0.908 | 0.850 | 0.967 | 0.954 | 0.910 | 0.999 |
| OLAH, RETN | | | | | | | | | |
| RSAD2, SIGLEC1, ISG15, SLC1A2, OLAH, | 0.863 | 0.854 | 0.873 | 0.911 | 0.853 | 0.968 | 0.958 | 0.916 | 0.999 |
| RETN, MMP8 | | | | | | | | | |
| OAS1, IFIT1, IFI44L, SIGLEC1, SLC1A2, IL1R2, | 0.863 | 0.854 | 0.873 | 0.914 | 0.859 | 0.968 | 0.963 | 0.925 | 1 |
| OLAH | | | | | | | | | |
| RSAD2, HERC6, SLC1A2, IL1R2, FAM20A, | 0.863 | 0.854 | 0.873 | 0.915 | 0.862 | 0.968 | 0.929 | 0.840 | 1 |
| RETN, MMP8 | | | | | | | | | |
| IFI44L, SIGLEC1, ISG15, HERC6, IL1R2, OLAH, | 0.863 | 0.854 | 0.873 | 0.903 | 0.839 | 0.966 | 0.959 | 0.917 | 1.000 |
| RETN | | | | | | | | | |
| OAS1, IFI44L, SIGLEC1, HERC6, SLC1A2, | 0.863 | 0.853 | 0.874 | 0.912 | 0.858 | 0.966 | 0.958 | 0.916 | 1.000 |
| IL1R2, OLAH | | | | | | | | | |
| RSAD2, OAS1, IFI44L, SIGLEC1, SLC1A2, | 0.863 | 0.853 | 0.873 | 0.903 | 0.841 | 0.966 | 0.943 | 0.870 | 1 |
| FAM20A, MMP8 | | | | | | | | | |
| IFI27, IFIT1, IFI44L, ISG15, HERC6, SLC1A2, | 0.863 | 0.853 | 0.874 | 0.905 | 0.847 | 0.964 | 0.932 | 0.848 | 1 |
| FAM20A | | | | | | | | | |
| RSAD2, IFIT1, IFI44L, SLC1A2, IL1R2, | 0.863 | 0.854 | 0.873 | 0.911 | 0.858 | 0.964 | 0.928 | 0.842 | 1 |
| FAM20A, MMP8 | | | | | | | | | |
| RSAD2, SLC1A2, IL1R2, OLAH, FAM20A, | 0.863 | 0.854 | 0.873 | 0.914 | 0.861 | 0.967 | 0.933 | 0.851 | 1 |
| RETN, MMP8 | | | | | | | | | |
| OAS1, IFI44L, SIGLEC1, SLC1A2, OLAH, | 0.863 | 0.853 | 0.873 | 0.911 | 0.853 | 0.969 | 0.959 | 0.915 | 1 |
| RETN, MMP8 | | | | | | | | | |
| RSAD2, OAS1, SIGLEC1, ISG15, SLC1A2, | 0.863 | 0.853 | 0.874 | 0.903 | 0.839 | 0.966 | 0.947 | 0.874 | 1 |
| FAM20A, RETN | | | | | | | | | |
| RSAD2, ISG15, HERC6, SLC1A2, OLAH, | 0.863 | 0.853 | 0.873 | 0.911 | 0.854 | 0.968 | 0.930 | 0.849 | 1 |
| FAM20A, RETN | | | | | | | | | |
| RSAD2, IFIT1, HERC6, IL1R2, OLAH, FAM20A, | 0.863 | 0.853 | 0.873 | 0.906 | 0.845 | 0.967 | 0.938 | 0.864 | 1 |
| RETN | | | | | | | | | |
| RSAD2, OAS1, IFI44L, ISG15, SLC1A2, OLAH, | 0.863 | 0.853 | 0.874 | 0.910 | 0.851 | 0.969 | 0.922 | 0.844 | 1.000 |
| FAM20A | | | | | | | | | |
| RSAD2, IFI27, IFIT1, HERC6, SLC1A2, | 0.863 | 0.853 | 0.873 | 0.911 | 0.855 | 0.967 | 0.928 | 0.845 | 1 |
| FAM20A, MMP8 | | | | | | | | | |
| OAS1, IFI44L, SIGLEC1, HERC6, SLC1A2, | 0.863 | 0.853 | 0.873 | 0.913 | 0.857 | 0.969 | 0.950 | 0.900 | 1 |
| OLAH, RETN | | | | | | | | | |
| RSAD2, IFI44L, SIGLEC1, ISG15, HERC6, | 0.863 | 0.854 | 0.873 | 0.902 | 0.841 | 0.963 | 0.959 | 0.918 | 1.000 |
| IL1R2, OLAH | | | | | | | | | |
| IFI27, IFI44L, ISG15, HERC6, SLC1A2, | 0.863 | 0.852 | 0.874 | 0.906 | 0.846 | 0.966 | 0.930 | 0.847 | 1 |
| FAM20A, MMP8 | | | | | | | | | |
| RSAD2, OAS1, ISG15, HERC6, OLAH, FAM20A, | 0.863 | 0.853 | 0.873 | 0.905 | 0.843 | 0.967 | 0.942 | 0.875 | 1 |
| RETN | | | | | | | | | |
| RSAD2, IFIT1, IFI44L, ISG15, HERC6, FAM20A, | 0.863 | 0.853 | 0.873 | 0.906 | 0.845 | 0.967 | 0.922 | 0.842 | 1 |
| RETN | | | | | | | | | |
| RSAD2, IFIT1, IFI44L, SIGLEC1, SLC1A2, | 0.863 | 0.854 | 0.872 | 0.911 | 0.855 | 0.967 | 0.965 | 0.929 | 1 |
| IL1R2, OLAH | | | | | | | | | |
| OAS1, IFIT1, IFI44L, SIGLEC1, HERC6, RETN, | 0.863 | 0.854 | 0.873 | 0.900 | 0.833 | 0.966 | 0.923 | 0.862 | 0.984 |
| MMP8 | | | | | | | | | |
| RSAD2, SIGLEC1, ISG15, HERC6, SLC1A2, | 0.863 | 0.853 | 0.873 | 0.912 | 0.855 | 0.969 | 0.948 | 0.900 | 0.995 |
| OLAH, RETN | | | | | | | | | |
| RSAD2, OAS1, IFI44L, SLC1A2, IL1R2, | 0.863 | 0.853 | 0.873 | 0.915 | 0.863 | 0.966 | 0.927 | 1.837 | 1 |
| FAM20A, MMP8 | | | | | | | | | |
| IFI44L, SIGLEC1, ISG15, SLC1A2, IL1R2, | 0.863 | 0.855 | 0.872 | 0.912 | 0.857 | 0.966 | 0.968 | 0.934 | 1 |
| RETN, MMP8 | | | | | | | | | |
| RSAD2, IFI44L, SLC1A2, OLAH, FAM20A, | 0.863 | 0.853 | 0.873 | 0.913 | 0.856 | 0.971 | 0.930 | 0.859 | 1 |
| RETN, MMP8 | | | | | | | | | |
| RSAD2, OAS1, IFIT1, SLC1A2, IL1R2, OLAH, | 0.863 | 0.854 | 0.873 | 0.911 | 0.854 | 0.968 | 0.933 | 0.853 | 1 |
| FAM20A | | | | | | | | | |
| IFIT1, IFI44L, SIGLEC1, ISG15, HERC6, OLAH, | 0.863 | 0.853 | 0.873 | 0.900 | 0.837 | 0.962 | 0.945 | 0.895 | 0.994 |
| MMP8 | | | | | | | | | |
| IFI44L, SIGLEC1, ISG15, HERC6, SLC1A2, | 0.863 | 0.854 | 0.872 | 0.909 | 0.853 | 0.964 | 0.958 | 0.915 | 1 |
| IL1R2, RETN | | | | | | | | | |
| IFI27, OAS1, IFIT1, SIGLEC1, ISG15, SLC1A2, | 0.863 | 0.854 | 0.872 | 0.905 | 0.847 | 0.963 | 0.929 | 0.862 | 0.997 |
| MMP8 | | | | | | | | | |
| OAS1, IFIT1, IFI44L, SLC1A2, IL1R2, FAM20A, | 0.863 | 0.853 | 0.873 | 0.914 | 0.862 | 0.966 | 0.929 | 0.841 | 1 |
| MMP8 | | | | | | | | | |
| RSAD2, OAS1, SLC1A2, IL1R2, OLAH, | 0.863 | 0.853 | 0.873 | 0.911 | 0.855 | 0.968 | 0.930 | 0.847 | 1 |
| FAM20A, RETN | | | | | | | | | |
| OAS1, IFI44L, SIGLEC1, ISG15, FAM20A, | 0.863 | 0.852 | 0.874 | 0.902 | 0.833 | 0.971 | 0.959 | 0.899 | 1 |
| RETN, MMP8 | | | | | | | | | |
| RSAD2, OAS1, IFI44L, HERC6, OLAH, | 0.863 | 0.852 | 0.874 | 0.904 | 0.842 | 0.966 | 0.941 | 0.877 | 1 |
| FAM20A, MMP8 | | | | | | | | | |
| RSAD2, OAS1, IFIT1, IL1R2, FAM20A, RETN, | 0.863 | 0.853 | 0.873 | 0.907 | 0.845 | 0.970 | 0.947 | 0.875 | 1 |
| MMP8 | | | | | | | | | |
| IFIT1, ISG15, HERC6, SLC1A2, OLAH, | 0.863 | 0.853 | 0.873 | 0.910 | 0.854 | 0.966 | 0.932 | 0.853 | 1 |
| FAM20A, MMP8 | | | | | | | | | |
| OAS1, IFIT1, HERC6, SLC1A2, IL1R2, FAM20A, | 0.863 | 0.853 | 0.873 | 0.911 | 0.857 | 0.966 | 0.930 | 0.846 | 1 |
| RETN | | | | | | | | | |
| RSAD2, OAS1, HERC6, OLAH, FAM20A, RETN, | 0.863 | 0.853 | 0.873 | 0.904 | 0.841 | 0.967 | 0.945 | 0.878 | 1 |
| MMP8 | | | | | | | | | |
| RSAD2, OAS1, IFIT1, HERC6, SLC1A2, OLAH, | 0.863 | 0.853 | 0.873 | 0.910 | 0.854 | 0.966 | 0.932 | 0.852 | 1 |
| FAM20A | | | | | | | | | |
| IFIT1, ISG15, HERC6, SLC1A2, OLAH, | 0.863 | 0.853 | 0.873 | 0.911 | 0.854 | 0.968 | 0.930 | 0.849 | 1 |
| FAM20A, RETN | | | | | | | | | |
| IFI44L, SIGLEC1, ISG15, HERC6, SLC1A2, | 0.863 | 0.854 | 0.872 | 0.907 | 0.853 | 0.962 | 0.964 | 0.927 | 1 |
| IL1R2, MMP8 | | | | | | | | | |
| RSAD2, IFIT1, IFI44L, SIGLEC1, HERC6, | 0.863 | 0.854 | 0.872 | 0.901 | 0.836 | 0.966 | 0.938 | 0.885 | 0.991 |
| OLAH, RETN | | | | | | | | | |
| RSAD2, IFI27, ISG15, SLC1A2, FAM20A, RETN, | 0.863 | 0.852 | 0.873 | 0.910 | 0.849 | 0.972 | 0.939 | 0.858 | 1 |
| MMP8 | | | | | | | | | |
| IFIT1, IFI44L, SIGLEC1, SLC1A2, IL1R2, OLAH, | 0.863 | 0.854 | 0.872 | 0.915 | 0.860 | 0.970 | 0.959 | 0.917 | 1 |
| RETN | | | | | | | | | |
| RSAD2, IFI44L, SIGLEC1, ISG15, FAM20A, | 0.863 | 0.853 | 0.873 | 0.903 | 0.836 | 0.970 | 0.953 | 0.894 | 1 |
| RETN, MMP8 | | | | | | | | | |
| RSAD2, OAS1, IFI44L, SIGLEC1, SLC1A2, | 0.863 | 0.853 | 0.873 | 0.912 | 0.856 | 0.968 | 0.967 | 0.930 | 1 |
| IL1R2, OLAH | | | | | | | | | |
| RSAD2, IFI27, OAS1, IFIT1, IFI44L, ISG15, | 0.863 | 0.853 | 0.873 | 0.905 | 0.842 | 0.968 | 0.953 | 0.892 | 1 |
| RETN | | | | | | | | | |
| RSAD2, IFI44L, SIGLEC1, ISG15, HERC6, | 0.863 | 0.853 | 0.872 | 0.911 | 0.855 | 0.967 | 0.946 | 0.896 | 0.995 |
| SLC1A2, OLAH | | | | | | | | | |
| RSAD2, OAS1, HERC6, SLC1A2, OLAH, | 0.863 | 0.853 | 0.873 | 0.911 | 0.854 | 0.967 | 0.933 | 0.852 | 1 |
| FAM20A, RETN | | | | | | | | | |
| RSAD2, SIGLEC1, ISG15, HERC6, IL1R2, | 0.863 | 0.853 | 0.873 | 0.904 | 0.842 | 0.966 | 0.958 | 0.915 | 1.000 |
| OLAH, RETN | | | | | | | | | |
| OAS1, IFIT1, SIGLEC1, SLC1A2, IL1R2, RETN, | 0.863 | 0.853 | 0.872 | 0.912 | 0.858 | 0.966 | 0.970 | 0.937 | 1 |
| MMP8 | | | | | | | | | |
| SIGLEC1, ISG15, HERC6, SLC1A2, IL1R2, | 0.863 | 0.854 | 0.872 | 0.909 | 0.853 | 0.965 | 0.970 | 0.937 | 1 |
| RETN, MMP8 | | | | | | | | | |
| RSAD2, IFIT1, ISG15, HERC6, OLAH, FAM20A, | 0.863 | 0.853 | 0.873 | 0.905 | 0.843 | 0.966 | 0.942 | 0.875 | 1 |
| RETN | | | | | | | | | |
| RSAD2, IFIT1, IFI44L, SIGLEC1, ISG15, | 0.863 | 0.854 | 0.872 | 0.913 | 0.857 | 0.969 | 0.947 | 0.897 | 0.996 |
| SLC1A2, OLAH | | | | | | | | | |
| RSAD2, IFI27, OAS1, HERC6, SLC1A2, | 0.863 | 0.852 | 0.873 | 0.913 | 0.858 | 0.968 | 0.924 | 0.839 | 1 |
| FAM20A, MMP8 | | | | | | | | | |
| OAS1, IFIT1, IFI44L, SIGLEC1, HERC6, OLAH, | 0.863 | 0.853 | 0.872 | 0.899 | 0.838 | 0.961 | 0.939 | 0.886 | 0.992 |
| MMP8 | | | | | | | | | |
| RSAD2, OAS1, IFIT1, SIGLEC1, HERC6, | 0.863 | 0.853 | 0.872 | 0.909 | 0.850 | 0.967 | 0.934 | 0.876 | 0.992 |
| SLC1A2, RETN | | | | | | | | | |
| RSAD2, IFI44L, SIGLEC1, ISG15, SLC1A2, | 0.863 | 0.853 | 0.873 | 0.902 | 0.839 | 0.965 | 0.945 | 0.873 | 1 |
| FAM20A, MMP8 | | | | | | | | | |
| RSAD2, OAS1, IFI44L, ISG15, HERC6, OLAH, | 0.863 | 0.852 | 0.873 | 0.904 | 0.843 | 0.966 | 0.941 | 0.877 | 1 |
| FAM20A | | | | | | | | | |
| RSAD2, OAS1, IFIT1, HERC6, SLC1A2, IL1R2, | 0.863 | 0.853 | 0.872 | 0.913 | 0.859 | 0.966 | 0.930 | 0.845 | 1 |
| FAM20A | | | | | | | | | |
| RSAD2, OAS1, IFI44L, SIGLEC1, ISG15, | 0.863 | 0.852 | 0.873 | 0.897 | 0.826 | 0.967 | 0.953 | 0.895 | 1 |
| FAM20A, RETN | | | | | | | | | |
| IFIT1, IFI44L, SIGLEC1, ISG15, HERC6, | 0.863 | 0.854 | 0.872 | 0.908 | 0.854 | 0.963 | 0.960 | 0.919 | 1 |
| SLC1A2, IL1R2 | | | | | | | | | |
| IFI44L, SIGLEC1, ISG15, IL1R2, OLAH, RETN, | 0.863 | 0.853 | 0.872 | 0.902 | 0.840 | 0.964 | 0.965 | 0.929 | 1 |
| MMP8 | | | | | | | | | |
| RSAD2, OAS1, IFI44L, ISG15, IL1R2, FAM20A, | 0.863 | 0.852 | 0.873 | 0.906 | 0.845 | 0.967 | 0.937 | 0.858 | 1 |
| MMP8 | | | | | | | | | |
| RSAD2, OAS1, IFIT1, SLC1A2, OLAH, | 0.863 | 0.853 | 0.872 | 0.909 | 0.851 | 0.967 | 0.941 | 0.872 | 1 |
| FAM20A, MMP8 | | | | | | | | | |
| RSAD2, IFIT1, IFI44L, HERC6, OLAH, | 0.863 | 0.853 | 0.872 | 0.906 | 0.844 | 0.968 | 0.933 | 0.863 | 1 |
| FAM20A, RETN | | | | | | | | | |
| OAS1, IFI44L, SLC1A2, IL1R2, OLAH, | 0.863 | 0.852 | 0.873 | 0.912 | 0.856 | 0.967 | 0.928 | 0.848 | 1 |
| FAM20A, RETN | | | | | | | | | |
| RSAD2, IFI27, IFIT1, IFI44L, ISG15, FAM20A, | 0.863 | 0.852 | 0.873 | 0.899 | 0.832 | 0.966 | 0.937 | 0.860 | 1 |
| MMP8 | | | | | | | | | |
| RSAD2, OAS1, IFIT1, IFI44L, SIGLEC1, IL1R2, | 0.863 | 0.853 | 0.872 | 0.898 | 0.832 | 0.963 | 0.958 | 0.916 | 0.999 |
| OLAH | | | | | | | | | |
| OAS1, IFIT1, ISG15, SLC1A2, IL1R2, FAM20A, | 0.863 | 0.853 | 0.872 | 0.913 | 0.859 | 0.967 | 0.934 | 0.852 | 1 |
| MMP8 | | | | | | | | | |
| RSAD2, OAS1, IFI44L, SIGLEC1, HERC6, | 0.863 | 0.853 | 0.872 | 0.908 | 0.853 | 0.963 | 0.963 | 0.923 | 1 |
| SLC1A2, IL1R2 | | | | | | | | | |
| RSAD2, OAS1, SIGLEC1, HERC6, OLAH, | 0.863 | 0.852 | 0.873 | 0.898 | 0.831 | 0.965 | 0.959 | 0.918 | 1.000 |
| RETN, MMP8 | | | | | | | | | |
| RSAD2, IFIT1, IFI44L, ISG15, SLC1A2, OLAH, | 0.863 | 0.853 | 0.872 | 0.909 | 0.850 | 0.968 | 0.922 | 0.845 | 0.998 |
| FAM20A | | | | | | | | | |
| OAS1, IFI44L, SIGLEC1, ISG15, SLC1A2, IL1R2, | 0.863 | 0.853 | 0.872 | 0.913 | 0.858 | 0.968 | 0.962 | 0.924 | 1 |
| OLAH | | | | | | | | | |
| OAS1, IFIT1, SIGLEC1, ISG15, SLC1A2, IL1R2, | 0.863 | 0.853 | 0.872 | 0.909 | 0.854 | 0.964 | 0.965 | 0.927 | 1 |
| RETN | | | | | | | | | |
| OAS1, IFIT1, SIGLEC1, HERC6, SLC1A2, | 0.862 | 0.853 | 0.872 | 0.905 | 0.845 | 0.965 | 0.929 | 0.871 | 0.988 |
| RETN, MMP8 | | | | | | | | | |
| RSAD2, OAS1, IFI44L, SIGLEC1, ISG15, | 0.862 | 0.852 | 0.873 | 0.904 | 0.842 | 0.967 | 0.945 | 0.873 | 1 |
| SLC1A2, FAM20A | | | | | | | | | |
| RSAD2, OAS1, ISG15, IL1R2, OLAH, FAM20A, | 0.862 | 0.853 | 0.872 | 0.900 | 0.834 | 0.965 | 0.932 | 0.854 | 1 |
| RETN | | | | | | | | | |
| OAS1, IFI44L, SIGLEC1, ISG15, SLC1A2, | 0.862 | 0.853 | 0.872 | 0.913 | 0.857 | 0.969 | 0.950 | 0.900 | 1.000 |
| OLAH, RETN | | | | | | | | | |
| OAS1, ISG15, IL1R2, OLAH, FAM20A, RETN, | 0.862 | 0.853 | 0.872 | 0.907 | 0.845 | 0.969 | 0.939 | 0.862 | 1 |
| MMP8 | | | | | | | | | |
| RSAD2, IFIT1, IFI44L, SIGLEC1, SLC1A2, | 0.862 | 0.853 | 0.872 | 0.913 | 0.857 | 0.970 | 0.947 | 0.896 | 0.998 |
| OLAH, RETN | | | | | | | | | |
| RSAD2, IFIT1, ISG15, IL1R2, OLAH, FAM20A, | 0.862 | 0.853 | 0.872 | 0.905 | 0.843 | 0.967 | 0.945 | 0.874 | 1 |
| MMP8 | | | | | | | | | |
| RSAD2, OAS1, IFI44L, ISG15, HERC6, | 0.862 | 0.852 | 0.873 | 0.905 | 0.843 | 0.966 | 0.935 | 0.859 | 1 |
| FAM20A, RETN | | | | | | | | | |
| OAS1, IFI44L, SIGLEC1, HERC6, IL1R2, OLAH, | 0.862 | 0.852 | 0.873 | 0.904 | 0.842 | 0.966 | 0.955 | 0.911 | 0.999 |
| RETN | | | | | | | | | |
| OAS1, IFI44L, SIGLEC1, ISG15, HERC6, | 0.862 | 0.853 | 0.872 | 0.909 | 0.856 | 0.963 | 0.961 | 0.921 | 1 |
| SLC1A2, IL1R2 | | | | | | | | | |
| RSAD2, IFI27, IFI44L, SIGLEC1, ISG15, HERC6, | 0.862 | 0.854 | 0.871 | 0.901 | 0.843 | 0.960 | 0.905 | 0.830 | 0.981 |
| MMP8 | | | | | | | | | |
| RSAD2, IFI44L, ISG15, HERC6, OLAH, | 0.862 | 0.852 | 0.873 | 0.906 | 0.844 | 0.967 | 0.942 | 0.878 | 1 |
| FAM20A, RETN | | | | | | | | | |
| RSAD2, IFI44L, SIGLEC1, HERC6, SLC1A2, | 0.862 | 0.853 | 0.871 | 0.906 | 0.851 | 0.962 | 0.964 | 0.927 | 1 |
| IL1R2, MMP8 | | | | | | | | | |
| RSAD2, IFIT1, ISG15, SLC1A2, IL1R2, | 0.862 | 0.853 | 0.871 | 0.910 | 0.855 | 0.965 | 0.933 | 0.851 | 1 |
| FAM20A, MMP8 | | | | | | | | | |
| OAS1, SLC1A2, IL1R2, OLAH, FAM20A, RETN, | 0.862 | 0.853 | 0.872 | 0.915 | 0.863 | 0.968 | 0.932 | 0.850 | 1 |
| MMP8 | | | | | | | | | |
| RSAD2, IFIT1, SIGLEC1, ISG15, SLC1A2, | 0.862 | 0.853 | 0.872 | 0.914 | 0.857 | 0.970 | 0.953 | 0.907 | 1.000 |
| OLAH, RETN | | | | | | | | | |
| RSAD2, IFI44L, SIGLEC1, SLC1A2, IL1R2, | 0.862 | 0.853 | 0.872 | 0.913 | 0.857 | 0.968 | 0.965 | 0.928 | 1 |
| OLAH, RETN | | | | | | | | | |
| OAS1, SIGLEC1, ISG15, HERC6, OLAH, RETN, | 0.862 | 0.852 | 0.873 | 0.900 | 0.835 | 0.966 | 0.970 | 0.935 | 1 |
| MMP8 | | | | | | | | | |
| RSAD2, IFI44L, SIGLEC1, ISG15, SLC1A2, | 0.862 | 0.853 | 0.872 | 0.912 | 0.857 | 0.968 | 0.951 | 0.905 | 0.997 |
| OLAH, MMP8 | | | | | | | | | |
| RSAD2, OAS1, IFIT1, IFI44L, SLC1A2, OLAH, | 0.862 | 0.853 | 0.872 | 0.911 | 0.853 | 0.969 | 0.921 | 0.842 | 0.999 |
| FAM20A | | | | | | | | | |
| RSAD2, IFIT1, IFI44L, SIGLEC1, IL1R2, OLAH, | 0.862 | 0.853 | 0.871 | 0.902 | 0.841 | 0.962 | 0.960 | 0.919 | 1 |
| MMP8 | | | | | | | | | |
| IFI27, IFIT1, IFI44L, ISG15, SLC1A2, FAM20A, | 0.862 | 0.852 | 0.873 | 0.901 | 0.837 | 0.966 | 0.933 | 0.851 | 1 |
| MMP8 | | | | | | | | | |
| OAS1, IFIT1, IFI44L, ISG15, IL1R2, OLAH, | 0.862 | 0.853 | 0.872 | 0.901 | 0.835 | 0.967 | 0.948 | 0.880 | 1 |
| FAM20A | | | | | | | | | |
| OAS1, IFI44L, SLC1A2, OLAH, FAM20A, | 0.862 | 0.852 | 0.873 | 0.914 | 0.857 | 0.971 | 0.932 | 0.859 | 1 |
| RETN, MMP8 | | | | | | | | | |
| OAS1, IFIT1, SIGLEC1, ISG15, HERC6, OLAH, | 0.862 | 0.852 | 0.872 | 0.900 | 0.839 | 0.961 | 0.943 | 0.893 | 0.994 |
| MMP8 | | | | | | | | | |
| IFIT1, IFI44L, SIGLEC1, ISG15, IL1R2, OLAH, | 0.862 | 0.853 | 0.871 | 0.904 | 0.847 | 0.962 | 0.959 | 0.917 | 1 |
| MMP8 | | | | | | | | | |
| RSAD2, IFIT1, IFI44L, HERC6, IL1R2, FAM20A, | 0.862 | 0.853 | 0.872 | 0.907 | 0.846 | 0.968 | 0.936 | 0.855 | 1 |
| RETN | | | | | | | | | |
| IFIT1, IFI44L, ISG15, SLC1A2, IL1R2, FAM20A, | 0.862 | 0.853 | 0.872 | 0.908 | 0.853 | 0.963 | 0.926 | 0.839 | 1 |
| RETN | | | | | | | | | |
| IFIT1, ISG15, SLC1A2, IL1R2, OLAH, FAM20A, | 0.862 | 0.852 | 0.872 | 0.908 | 0.851 | 0.965 | 0.932 | 0.857 | 1 |
| RETN | | | | | | | | | |
| RSAD2, IFIT1, IFI44L, IL1R2, OLAH, FAM20A, | 0.862 | 0.853 | 0.872 | 0.905 | 0.844 | 0.967 | 0.952 | 0.892 | 1 |
| MMP8 | | | | | | | | | |
| RSAD2, IFI44L, SIGLEC1, HERC6, SLC1A2, | 0.862 | 0.852 | 0.872 | 0.913 | 0.856 | 0.969 | 0.946 | 0.896 | 0.995 |
| OLAH, RETN | | | | | | | | | |
| OAS1, IFIT1, ISG15, IL1R2, OLAH, FAM20A, | 0.862 | 0.852 | 0.872 | 0.900 | 0.833 | 0.966 | 0.941 | 0.868 | 1 |
| RETN | | | | | | | | | |
| OAS1, IFIT1, SLC1A2, IL1R2, OLAH, FAM20A, | 0.862 | 0.852 | 0.872 | 0.911 | 0.854 | 0.968 | 0.935 | 0.855 | 1 |
| RETN | | | | | | | | | |
| RSAD2, OAS1, HERC6, SLC1A2, IL1R2, | 0.862 | 0.852 | 0.872 | 0.911 | 0.856 | 0.965 | 0.929 | 0.840 | 1 |
| FAM20A, RETN | | | | | | | | | |
| OAS1, IFIT1, SLC1A2, IL1R2, FAM20A, RETN, | 0.862 | 0.852 | 0.872 | 0.911 | 0.856 | 0.967 | 0.937 | 0.858 | 1 |
| MMP8 | | | | | | | | | |
| RSAD2, OAS1, SIGLEC1, IL1R2, OLAH, RETN, | 0.862 | 0.852 | 0.873 | 0.901 | 0.837 | 0.964 | 0.968 | 0.935 | 1 |
| MMP8 | | | | | | | | | |
| OAS1, IFI44L, SIGLEC1, SLC1A2, IL1R2, | 0.862 | 0.852 | 0.872 | 0.914 | 0.858 | 0.969 | 0.962 | 0.922 | 1 |
| OLAH, RETN | | | | | | | | | |
| RSAD2, IFI44L, SIGLEC1, ISG15, SLC1A2, | 0.862 | 0.853 | 0.871 | 0.911 | 0.856 | 0.967 | 0.963 | 0.925 | 1 |
| IL1R2, OLAH | | | | | | | | | |
| RSAD2, OAS1, IFIT1, HERC6, OLAH, FAM20A, | 0.862 | 0.852 | 0.872 | 0.899 | 0.835 | 0.962 | 0.941 | 0.874 | 1 |
| MMP8 | | | | | | | | | |
| RSAD2, IFI44L, SIGLEC1, ISG15, OLAH, | 0.862 | 0.852 | 0.872 | 0.897 | 0.826 | 0.969 | 0.962 | 0.919 | 1 |
| FAM20A, RETN | | | | | | | | | |
| RSAD2, OAS1, IFIT1, IL1R2, OLAH, FAM20A, | 0.862 | 0.852 | 0.872 | 0.901 | 0.836 | 0.966 | 0.940 | 0.867 | 1 |
| RETN | | | | | | | | | |
| OAS1, IFI44L, HERC6, SLC1A2, IL1R2, | 0.862 | 0.852 | 0.872 | 0.915 | 0.864 | 0.967 | 0.927 | 0.840 | 1 |
| FAM20A, RETN | | | | | | | | | |
| RSAD2, IFI27, OAS1, IFIT1, SIGLEC1, SLC1A2, | 0.862 | 0.853 | 0.871 | 0.904 | 0.846 | 0.963 | 0.932 | 0.861 | 1 |
| MMP8 | | | | | | | | | |
| OAS1, IFI44L, ISG15, HERC6, OLAH, FAM20A, | 0.862 | 0.852 | 0.872 | 0.905 | 0.843 | 0.967 | 0.946 | 0.884 | 1 |
| RETN | | | | | | | | | |
| OAS1, IFIT1, SIGLEC1, ISG15, SLC1A2, IL1R2, | 0.862 | 0.853 | 0.871 | 0.910 | 0.856 | 0.964 | 0.967 | 0.932 | 1 |
| MMP8 | | | | | | | | | |
| RSAD2, IFIT1, SIGLEC1, SLC1A2, IL1R2, | 0.862 | 0.853 | 0.871 | 0.912 | 0.858 | 0.966 | 0.971 | 0.939 | 1 |
| RETN, MMP8 | | | | | | | | | |
| IFIT1, IFI44L, SIGLEC1, ISG15, SLC1A2, IL1R2, | 0.862 | 0.853 | 0.871 | 0.910 | 0.854 | 0.965 | 0.965 | 0.927 | 1 |
| RETN | | | | | | | | | |
| RSAD2, OAS1, IFIT1, IFI44L, ISG15, IL1R2, | 0.862 | 0.852 | 0.872 | 0.902 | 0.839 | 0.965 | 0.941 | 0.867 | 1 |
| FAM20A | | | | | | | | | |
| OAS1, IFIT1, ISG15, IL1R2, FAM20A, RETN, | 0.862 | 0.852 | 0.872 | 0.905 | 0.842 | 0.969 | 0.948 | 0.874 | 1 |
| MMP8 | | | | | | | | | |
| RSAD2, IFI44L, SIGLEC1, HERC6, IL1R2, | 0.862 | 0.852 | 0.872 | 0.900 | 0.836 | 0.965 | 0.958 | 0.916 | 0.999 |
| OLAH, RETN | | | | | | | | | |
| RSAD2, OAS1, IFI44L, SIGLEC1, IL1R2, OLAH, | 0.862 | 0.852 | 0.872 | 0.901 | 0.841 | 0.962 | 0.960 | 0.919 | 1 |
| MMP8 | | | | | | | | | |
| RSAD2, IFIT1, ISG15, HERC6, SLC1A2, OLAH, | 0.862 | 0.852 | 0.872 | 0.911 | 0.854 | 0.967 | 0.932 | 0.852 | 1 |
| FAM20A | | | | | | | | | |
| RSAD2, IFI44L, SIGLEC1, HERC6, SLC1A2, | 0.862 | 0.853 | 0.871 | 0.908 | 0.853 | 0.964 | 0.955 | 0.912 | 0.999 |
| IL1R2, RETN | | | | | | | | | |
| RSAD2, IFI27, SIGLEC1, ISG15, HERC6, | 0.862 | 0.853 | 0.871 | 0.903 | 0.846 | 0.959 | 0.926 | 0.859 | 0.993 |
| SLC1A2, MMP8 | | | | | | | | | |
| IFIT1, SIGLEC1, ISG15, SLC1A2, IL1R2, RETN, | 0.862 | 0.853 | 0.871 | 0.912 | 0.858 | 0.966 | 0.971 | 0.939 | 1 |
| MMP8 | | | | | | | | | |
| IFIT1, IFI44L, SIGLEC1, IL1R2, OLAH, RETN, | 0.862 | 0.852 | 0.872 | 0.902 | 0.840 | 0.964 | 0.964 | 0.927 | 1 |
| MMP8 | | | | | | | | | |
| IFI27, OAS1, IFIT1, IFI44L, SIGLEC1, SLC1A2, | 0.862 | 0.853 | 0.871 | 0.905 | 0.846 | 0.963 | 0.930 | 0.864 | 0.997 |
| MMP8 | | | | | | | | | |
| IFI27, OAS1, IFIT1, IFI44L, ISG15, FAM20A, | 0.862 | 0.851 | 0.873 | 0.898 | 0.83 | 0.965 | 0.934 | 0.854 | 1 |
| MMP8 | | | | | | | | | |
| IFI44L, ISG15, IL1R2, OLAH, FAM20A, RETN, | 0.862 | 0.852 | 0.871 | 0.905 | 0.843 | 0.966 | 0.948 | 0.886 | 1 |
| MMP8 | | | | | | | | | |
| OAS1, IFIT1, IFI44L, SIGLEC1, SLC1A2, IL1R2, | 0.862 | 0.853 | 0.871 | 0.910 | 0.855 | 0.965 | 0.962 | 0.922 | 1 |
| RETN | | | | | | | | | |
| RSAD2, OAS1, SIGLEC1, ISG15, SLC1A2, | 0.862 | 0.852 | 0.872 | 0.912 | 0.855 | 0.968 | 0.955 | 0.906 | 1 |
| OLAH, RETN | | | | | | | | | |
| OAS1, IFI44L, SIGLEC1, IL1R2, OLAH, RETN, | 0.862 | 0.852 | 0.872 | 0.903 | 0.841 | 0.965 | 0.963 | 0.925 | 1 |
| MMP8 | | | | | | | | | |
| RSAD2, OAS1, IFIT1, HERC6, OLAH, FAM20A, | 0.862 | 0.852 | 0.872 | 0.901 | 0.837 | 0.966 | 0.943 | 0.876 | 1 |
| RETN | | | | | | | | | |
| RSAD2, IFI44L, SIGLEC1, SLC1A2, OLAH, | 0.862 | 0.852 | 0.871 | 0.912 | 0.854 | 0.969 | 0.957 | 0.914 | 0.999 |
| RETN, MMP8 | | | | | | | | | |
| RSAD2, OAS1, IFI44L, SIGLEC1, ISG15, | 0.862 | 0.852 | 0.871 | 0.911 | 0.855 | 0.967 | 0.948 | 0.898 | 0.998 |
| SLC1A2, OLAH | | | | | | | | | |
| OAS1, SIGLEC1, ISG15, HERC6, SLC1A2, | 0.862 | 0.852 | 0.872 | 0.905 | 0.844 | 0.965 | 0.952 | 0.903 | 1 |
| RETN, MMP8 | | | | | | | | | |
| RSAD2, IFIT1, ISG15, SLC1A2, IL1R2, OLAH, | 0.862 | 0.853 | 0.871 | 0.908 | 0.850 | 0.966 | 0.933 | 0.855 | 1 |
| FAM20A | | | | | | | | | |
| OAS1, IFIT1, IFI44L, SIGLEC1, ISG15, IL1R2, | 0.862 | 0.852 | 0.871 | 0.901 | 0.838 | 0.965 | 0.953 | 0.909 | 0.997 |
| OLAH | | | | | | | | | |
| RSAD2, OAS1, IFIT1, SIGLEC1, IL1R2, OLAH, | 0.862 | 0.852 | 0.871 | 0.904 | 0.845 | 0.963 | 0.963 | 0.925 | 1 |
| MMP8 | | | | | | | | | |
| OAS1, IFI44L, HERC6, OLAH, FAM20A, RETN, | 0.862 | 0.851 | 0.872 | 0.904 | 0.839 | 0.968 | 0.948 | 0.885 | 1 |
| MMP8 | | | | | | | | | |
| OAS1, IFIT1, IFI44L, SIGLEC1, SLC1A2, IL1R2, | 0.862 | 0.853 | 0.870 | 0.908 | 0.854 | 0.962 | 0.964 | 0.926 | 1 |
| MMP8 | | | | | | | | | |
| RSAD2, OAS1, IFIT1, SIGLEC1, HERC6, OLAH, | 0.862 | 0.852 | 0.871 | 0.899 | 0.837 | 0.961 | 0.940 | 0.888 | 0.993 |
| MMP8 | | | | | | | | | |
| IFI27, OAS1, IFIT1, IFI44L, ISG15, HERC6, | 0.862 | 0.853 | 0.870 | 0.901 | 0.844 | 0.957 | 0.893 | 0.813 | 0.974 |
| MMP8 | | | | | | | | | |
| RSAD2, OAS1, IFIT1, SIGLEC1, HERC6, RETN, | 0.862 | 0.852 | 0.872 | 0.899 | 0.832 | 0.966 | 0.926 | 0.866 | 0.986 |
| MMP8 | | | | | | | | | |
| RSAD2, IFIT1, IFI44L, SIGLEC1, HERC6, | 0.862 | 0.853 | 0.870 | 0.906 | 0.851 | 0.962 | 0.960 | 0.920 | 1.000 |
| SLC1A2, IL1R2 | | | | | | | | | |
| RSAD2, IFIT1, SLC1A2, IL1R2, FAM20A, | 0.862 | 0.852 | 0.871 | 0.911 | 0.855 | 0.966 | 0.937 | 0.859 | 1 |
| RETN, MMP8 | | | | | | | | | |
| IFIT1, IFI44L, SIGLEC1, ISG15, IL1R2, RETN, | 0.862 | 0.852 | 0.871 | 0.902 | 0.839 | 0.965 | 0.965 | 0.929 | 1 |
| MMP8 | | | | | | | | | |
| IFIT1, IFI44L, SIGLEC1, SLC1A2, IL1R2, RETN, | 0.862 | 0.853 | 0.871 | 0.912 | 0.858 | 0.966 | 0.967 | 0.932 | 1 |
| MMP8 | | | | | | | | | |
| OAS1, ISG15, SLC1A2, IL1R2, OLAH, FAM20A, | 0.862 | 0.851 | 0.872 | 0.912 | 0.856 | 0.968 | 0.926 | 0.842 | 1 |
| RETN | | | | | | | | | |
| IFIT1, ISG15, SLC1A2, IL1R2, FAM20A, RETN, | 0.862 | 0.852 | 0.871 | 0.911 | 0.856 | 0.966 | 0.938 | 0.861 | 1 |
| MMP8 | | | | | | | | | |
| RSAD2, IFI27, OAS1, SIGLEC1, ISG15, SLC1A2, | 0.862 | 0.852 | 0.871 | 0.905 | 0.846 | 0.964 | 0.937 | 0.872 | 1 |
| MMP8 | | | | | | | | | |
| RSAD2, IFI44L, SIGLEC1, ISG15, HERC6, | 0.862 | 0.852 | 0.871 | 0.908 | 0.853 | 0.963 | 0.959 | 0.918 | 1.000 |
| SLC1A2, IL1R2 | | | | | | | | | |
| RSAD2, OAS1, IFIT1, SIGLEC1, ISG15, | 0.861 | 0.852 | 0.871 | 0.907 | 0.851 | 0.963 | 0.968 | 0.932 | 1 |
| SLC1A2, IL1R2 | | | | | | | | | |
| RSAD2, OAS1, IFIT1, IFI44L, SIGLEC1, IL1R2, | 0.861 | 0.852 | 0.871 | 0.901 | 0.840 | 0.962 | 0.961 | 0.921 | 1 |
| MMP8 | | | | | | | | | |
| OAS1, IFI44L, SIGLEC1, HERC6, OLAH, RETN, | 0.861 | 0.851 | 0.872 | 0.898 | 0.831 | 0.965 | 0.962 | 0.924 | 1 |
| MMP8 | | | | | | | | | |
| RSAD2, ISG15, SLC1A2, IL1R2, OLAH, | 0.861 | 0.852 | 0.871 | 0.911 | 0.855 | 0.967 | 0.932 | 0.850 | 1 |
| FAM20A, RETN | | | | | | | | | |
| OAS1, IFIT1, IFI44L, SIGLEC1, ISG15, IL1R2, | 0.861 | 0.852 | 0.870 | 0.900 | 0.839 | 0.961 | 0.958 | 0.916 | 1.000 |
| MMP8 | | | | | | | | | |
| RSAD2, IFI44L, SIGLEC1, ISG15, SLC1A2, | 0.861 | 0.851 | 0.871 | 0.904 | 0.842 | 0.966 | 0.946 | 0.874 | 1 |
| FAM20A, RETN | | | | | | | | | |
| RSAD2, IFI44L, HERC6, SLC1A2, IL1R2, | 0.861 | 0.851 | 0.872 | 0.915 | 0.864 | 0.967 | 0.924 | 0.836 | 1 |
| FAM20A, RETN | | | | | | | | | |
| OAS1, IFI44L, ISG15, SLC1A2, OLAH, | 0.861 | 0.851 | 0.872 | 0.908 | 0.849 | 0.968 | 0.927 | 0.852 | 1 |
| FAM20A, RETN | | | | | | | | | |
| IFIT1, IFI44L, SIGLEC1, ISG15, SLC1A2, IL1R2, | 0.861 | 0.853 | 0.870 | 0.908 | 0.854 | 0.962 | 0.964 | 0.926 | 1 |
| MMP8 | | | | | | | | | |
| RSAD2, OAS1, IFI44L, SIGLEC1, SLC1A2, | 0.861 | 0.852 | 0.871 | 0.911 | 0.854 | 0.968 | 0.955 | 0.906 | 1 |
| OLAH, RETN | | | | | | | | | |
| IFI27, IFIT1, IFI44L, SIGLEC1, ISG15, SLC1A2, | 0.861 | 0.852 | 0.871 | 0.902 | 0.843 | 0.961 | 0.926 | 0.857 | 0.996 |
| MMP8 | | | | | | | | | |
| OAS1, IFIT1, ISG15, SLC1A2, IL1R2, FAM20A, | 0.861 | 0.851 | 0.871 | 0.908 | 0.851 | 0.964 | 0.936 | 0.855 | 1 |
| RETN | | | | | | | | | |
| RSAD2, OAS1, IFIT1, SIGLEC1, IL1R2, RETN, | 0.861 | 0.851 | 0.871 | 0.902 | 0.839 | 0.965 | 0.968 | 0.935 | 1 |
| MMP8 | | | | | | | | | |
| IFI27, OAS1, IFIT1, IFI44L, ISG15, RETN, | 0.861 | 0.851 | 0.872 | 0.904 | 0.840 | 0.967 | 0.951 | 0.888 | 1 |
| MMP8 | | | | | | | | | |
| OAS1, IFIT1, IFI44L, ISG15, IL1R2, FAM20A, | 0.861 | 0.851 | 0.871 | 0.904 | 0.841 | 0.967 | 0.943 | 0.868 | 1 |
| MMP8 | | | | | | | | | |
| RSAD2, IFI27, IFI44L, SIGLEC1, HERC6, | 0.861 | 0.852 | 0.870 | 0.906 | 0.851 | 0.961 | 0.923 | 0.854 | 0.991 |
| SLC1A2, MMP8 | | | | | | | | | |
| IFIT1, SIGLEC1, ISG15, IL1R2, OLAH, RETN, | 0.861 | 0.851 | 0.871 | 0.903 | 0.841 | 0.965 | 0.968 | 0.935 | 1 |
| MMP8 | | | | | | | | | |
| OAS1, IFIT1, HERC6, SLC1A2, OLAH, | 0.861 | 0.851 | 0.872 | 0.910 | 0.854 | 0.967 | 0.930 | 0.850 | 1 |
| FAM20A, RETN | | | | | | | | | |
| RSAD2, OAS1, IFIT1, IFI44L, SIGLEC1, IL1R2, | 0.861 | 0.852 | 0.871 | 0.897 | 0.830 | 0.963 | 0.961 | 0.921 | 1 |
| RETN | | | | | | | | | |
| OAS1, IFI44L, SIGLEC1, ISG15, SLC1A2, | 0.861 | 0.851 | 0.871 | 0.901 | 0.837 | 0.965 | 0.947 | 0.875 | 1 |
| FAM20A, RETN | | | | | | | | | |
| IFI27, IFI44L, SIGLEC1, ISG15, HERC6, | 0.861 | 0.852 | 0.870 | 0.902 | 0.845 | 0.959 | 0.924 | 0.855 | 0.993 |
| SLC1A2, MMP8 | | | | | | | | | |
| OAS1, IFIT1, IFI44L, SLC1A2, OLAH, FAM20A, | 0.861 | 0.851 | 0.871 | 0.911 | 0.852 | 0.969 | 0.924 | 0.848 | 1.000 |
| RETN | | | | | | | | | |
| OAS1, IFIT1, IFI44L, SIGLEC1, ISG15, HERC6, | 0.861 | 0.851 | 0.871 | 0.900 | 0.839 | 0.962 | 0.941 | 0.890 | 0.993 |
| OLAH | | | | | | | | | |
| RSAD2, IFI44L, SIGLEC1, ISG15, SLC1A2, | 0.861 | 0.852 | 0.871 | 0.913 | 0.856 | 0.969 | 0.948 | 0.898 | 0.998 |
| OLAH, RETN | | | | | | | | | |
| IFI44L, SIGLEC1, ISG15, HERC6, OLAH, RETN, | 0.861 | 0.851 | 0.871 | 0.896 | 0.827 | 0.965 | 0.963 | 0.925 | 1 |
| MMP8 | | | | | | | | | |
| OAS1, SIGLEC1, ISG15, SLC1A2, IL1R2, RETN, | 0.861 | 0.852 | 0.870 | 0.912 | 0.858 | 0.966 | 0.971 | 0.938 | 1 |
| MMP8 | | | | | | | | | |
| RSAD2, OAS1, ISG15, SLC1A2, OLAH, | 0.861 | 0.851 | 0.871 | 0.909 | 0.851 | 0.967 | 0.936 | 0.861 | 1 |
| FAM20A, MMP8 | | | | | | | | | |
| RSAD2, OAS1, SIGLEC1, ISG15, IL1R2, OLAH, | 0.861 | 0.851 | 0.871 | 0.904 | 0.846 | 0.963 | 0.963 | 0.926 | 1 |
| MMP8 | | | | | | | | | |
| OAS1, IFIT1, IFI44L, ISG15, SLC1A2, IL1R2, | 0.861 | 0.851 | 0.871 | 0.907 | 0.850 | 0.964 | 0.926 | 0.839 | 1 |
| FAM20A | | | | | | | | | |
| RSAD2, OAS1, IFI44L, SIGLEC1, ISG15, | 0.861 | 0.850 | 0.872 | 0.897 | 0.828 | 0.967 | 0.951 | 0.888 | 1 |
| FAM20A, MMP8 | | | | | | | | | |
| RSAD2, IFIT1, IFI44L, SLC1A2, OLAH, | 0.861 | 0.851 | 0.871 | 0.909 | 0.850 | 0.968 | 0.921 | 0.844 | 0.997 |
| FAM20A, RETN | | | | | | | | | |
| RSAD2, OAS1, IFI44L, SIGLEC1, ISG15, IL1R2, | 0.861 | 0.851 | 0.870 | 0.897 | 0.831 | 0.963 | 0.958 | 0.916 | 1.000 |
| OLAH | | | | | | | | | |
| RSAD2, OAS1, IFI44L, HERC6, OLAH, | 0.861 | 0.851 | 0.871 | 0.903 | 0.839 | 0.966 | 0.938 | 0.871 | 1 |
| FAM20A, RETN | | | | | | | | | |
| RSAD2, OAS1, ISG15, HERC6, OLAH, FAM20A, | 0.861 | 0.851 | 0.871 | 0.901 | 0.840 | 0.963 | 0.941 | 0.872 | 1 |
| MMP8 | | | | | | | | | |
| OAS1, IFIT1, SIGLEC1, ISG15, IL1R2, OLAH, | 0.861 | 0.852 | 0.870 | 0.905 | 0.846 | 0.964 | 0.957 | 0.915 | 0.998 |
| MMP8 | | | | | | | | | |
| RSAD2, IFIT1, IFI44L, SIGLEC1, ISG15, HERC6, | 0.861 | 0.851 | 0.870 | 0.899 | 0.834 | 0.964 | 0.926 | 0.867 | 0.986 |
| RETN | | | | | | | | | |
| RSAD2, IFIT1, IFI44L, SIGLEC1, IL1R2, RETN, | 0.861 | 0.851 | 0.870 | 0.900 | 0.835 | 0.964 | 0.967 | 0.933 | 1 |
| MMP8 | | | | | | | | | |
| OAS1, IFI44L, IL1R2, OLAH, FAM20A, RETN, | 0.861 | 0.851 | 0.871 | 0.905 | 0.842 | 0.968 | 0.947 | 0.878 | 1 |
| MMP8 | | | | | | | | | |
| OAS1, IFIT1, IFI44L, IL1R2, FAM20A, RETN, | 0.861 | 0.851 | 0.871 | 0.907 | 0.845 | 0.970 | 0.946 | 0.871 | 1 |
| MMP8 | | | | | | | | | |
| RSAD2, OAS1, IFIT1, SIGLEC1, SLC1A2, | 0.861 | 0.851 | 0.870 | 0.909 | 0.853 | 0.965 | 0.970 | 0.933 | 1 |
| IL1R2, RETN | | | | | | | | | |
| OAS1, IFIT1, SIGLEC1, ISG15, IL1R2, RETN, | 0.861 | 0.851 | 0.871 | 0.901 | 0.838 | 0.965 | 0.968 | 0.935 | 1 |
| MMP8 | | | | | | | | | |
| RSAD2, OAS1, SIGLEC1, SLC1A2, IL1R2, | 0.861 | 0.851 | 0.870 | 0.912 | 0.859 | 0.966 | 0.973 | 0.941 | 1 |
| RETN, MMP8 | | | | | | | | | |
| OAS1, IFIT1, IFI44L, SIGLEC1, ISG15, SLC1A2, | 0.861 | 0.852 | 0.870 | 0.907 | 0.851 | 0.963 | 0.964 | 0.927 | 1 |
| IL1R2 | | | | | | | | | |
| RSAD2, OAS1, IFIT1, ISG15, HERC6, FAM20A, | 0.861 | 0.851 | 0.871 | 0.900 | 0.837 | 0.963 | 0.935 | 0.858 | 1 |
| RETN | | | | | | | | | |
| RSAD2, IFI44L, HERC6, OLAH, FAM20A, | 0.861 | 0.851 | 0.871 | 0.906 | 0.843 | 0.969 | 0.941 | 0.876 | 1 |
| RETN, MMP8 | | | | | | | | | |
| OAS1, IFI44L, ISG15, SLC1A2, IL1R2, FAM20A, | 0.861 | 0.851 | 0.871 | 0.913 | 0.862 | 0.965 | 0.927 | 0.834 | 1 |
| MMP8 | | | | | | | | | |
| OAS1, IFIT1, ISG15, HERC6, OLAH, FAM20A, | 0.861 | 0.851 | 0.871 | 0.900 | 0.837 | 0.963 | 0.942 | 0.873 | 1 |
| MMP8 | | | | | | | | | |
| RSAD2, OAS1, IFIT1, ISG15, SLC1A2, IL1R2, | 0.861 | 0.851 | 0.870 | 0.906 | 0.848 | 0.964 | 0.936 | 0.855 | 1 |
| FAM20A | | | | | | | | | |
| RSAD2, IFI44L, ISG15, SLC1A2, IL1R2, | 0.861 | 0.851 | 0.871 | 0.912 | 0.860 | 0.963 | 0.927 | 0.838 | 1 |
| FAM20A, MMP8 | | | | | | | | | |
| RSAD2, IFIT1, SLC1A2, IL1R2, OLAH, | 0.861 | 0.851 | 0.871 | 0.909 | 0.852 | 0.966 | 0.935 | 0.859 | 1 |
| FAM20A, RETN | | | | | | | | | |
| RSAD2, OAS1, IFIT1, SIGLEC1, ISG15, IL1R2, | 0.861 | 0.851 | 0.870 | 0.900 | 0.836 | 0.963 | 0.959 | 0.917 | 1 |
| OLAH | | | | | | | | | |
| IFIT1, IFI44L, IL1R2, OLAH, FAM20A, RETN, | 0.861 | 0.851 | 0.871 | 0.906 | 0.844 | 0.968 | 0.950 | 0.888 | 1 |
| MMP8 | | | | | | | | | |
| RSAD2, OAS1, SIGLEC1, ISG15, HERC6, | 0.861 | 0.851 | 0.871 | 0.905 | 0.844 | 0.965 | 0.952 | 0.902 | 1 |
| SLC1A2, RETN | | | | | | | | | |
| RSAD2, OAS1, IFI44L, SLC1A2, OLAH, | 0.861 | 0.850 | 0.871 | 0.911 | 0.852 | 0.969 | 0.923 | 0.847 | 0.999 |
| FAM20A, RETN | | | | | | | | | |
| RSAD2, IFI44L, ISG15, IL1R2, OLAH, FAM20A, | 0.861 | 0.851 | 0.871 | 0.904 | 0.843 | 0.965 | 0.948 | 0.884 | 1 |
| MMP8 | | | | | | | | | |
| RSAD2, IFI27, OAS1, IFI44L, ISG15, HERC6, | 0.861 | 0.852 | 0.870 | 0.900 | 0.842 | 0.958 | 0.903 | 0.827 | 0.980 |
| MMP8 | | | | | | | | | |
| RSAD2, OAS1, IFIT1, IFI44L, SIGLEC1, ISG15, | 0.861 | 0.852 | 0.870 | 0.896 | 0.831 | 0.961 | 0.958 | 0.917 | 0.999 |
| IL1R2 | | | | | | | | | |
| OAS1, IFIT1, ISG15, SLC1A2, OLAH, FAM20A, | 0.861 | 0.851 | 0.871 | 0.908 | 0.850 | 0.967 | 0.938 | 0.866 | 1 |
| MMP8 | | | | | | | | | |
| RSAD2, IFIT1, HERC6, OLAH, FAM20A, RETN, | 0.861 | 0.851 | 0.871 | 0.900 | 0.836 | 0.964 | 0.948 | 0.884 | 1 |
| MMP8 | | | | | | | | | |
| RSAD2, IFI27, IFIT1, IFI44L, SLC1A2, FAM20A, | 0.861 | 0.850 | 0.871 | 0.903 | 0.839 | 0.967 | 0.934 | 0.853 | 1 |
| MMP8 | | | | | | | | | |
| RSAD2, IFI27, IFI44L, ISG15, SLC1A2, | 0.861 | 0.850 | 0.872 | 0.903 | 0.839 | 0.967 | 0.938 | 0.858 | 1 |
| FAM20A, MMP8 | | | | | | | | | |
| RSAD2, IFI44L, ISG15, HERC6, OLAH, | 0.861 | 0.850 | 0.871 | 0.902 | 0.840 | 0.964 | 0.940 | 0.876 | 1 |
| FAM20A, MMP8 | | | | | | | | | |
| IFI27, OAS1, IFIT1, IFI44L, ISG15, SLC1A2, | 0.861 | 0.851 | 0.870 | 0.904 | 0.841 | 0.967 | 0.945 | 0.874 | 1 |
| RETN | | | | | | | | | |
| IFI27, OAS1, IFI44L, SIGLEC1, ISG15, SLC1A2, | 0.861 | 0.851 | 0.870 | 0.905 | 0.846 | 0.963 | 0.934 | 0.867 | 1 |
| MMP8 | | | | | | | | | |
| RSAD2, IFIT1, HERC6, SLC1A2, IL1R2, | 0.861 | 0.851 | 0.871 | 0.912 | 0.858 | 0.967 | 0.932 | 0.846 | 1 |
| FAM20A, RETN | | | | | | | | | |
| IFIT1, IFI44L, SIGLEC1, ISG15, HERC6, RETN, | 0.861 | 0.851 | 0.871 | 0.896 | 0.829 | 0.964 | 0.932 | 0.875 | 0.988 |
| MMP8 | | | | | | | | | |
| RSAD2, OAS1, IFIT1, SIGLEC1, IL1R2, OLAH, | 0.861 | 0.851 | 0.870 | 0.897 | 0.831 | 0.964 | 0.959 | 0.916 | 1 |
| RETN | | | | | | | | | |
| OAS1, IFI44L, SIGLEC1, ISG15, IL1R2, OLAH, | 0.861 | 0.851 | 0.870 | 0.903 | 0.844 | 0.963 | 0.957 | 0.914 | 0.999 |
| MMP8 | | | | | | | | | |
| RSAD2, IFI27, OAS1, IFI44L, SIGLEC1, | 0.861 | 0.851 | 0.870 | 0.904 | 0.844 | 0.963 | 0.933 | 0.863 | 1 |
| SLC1A2, MMP8 | | | | | | | | | |
| RSAD2, OAS1, IFI44L, IL1R2, OLAH, FAM20A, | 0.861 | 0.850 | 0.871 | 0.900 | 0.835 | 0.966 | 0.941 | 0.870 | 1 |
| RETN | | | | | | | | | |
| RSAD2, OAS1, IFIT1, ISG15, HERC6, OLAH, | 0.861 | 0.850 | 0.871 | 0.901 | 0.839 | 0.964 | 0.937 | 0.867 | 1 |
| FAM20A | | | | | | | | | |
| RSAD2, IFI27, OAS1, IFI44L, SLC1A2, | 0.861 | 0.850 | 0.872 | 0.903 | 0.838 | 0.967 | 0.935 | 0.854 | 1 |
| FAM20A, MMP8 | | | | | | | | | |
| RSAD2, IFI27, OAS1, ISG15, HERC6, SLC1A2, | 0.861 | 0.850 | 0.871 | 0.908 | 0.850 | 0.966 | 0.927 | 0.843 | 1 |
| FAM20A | | | | | | | | | |
| RSAD2, OAS1, IFIT1, SIGLEC1, SLC1A2, | 0.861 | 0.851 | 0.870 | 0.908 | 0.854 | 0.962 | 0.971 | 0.938 | 1 |
| IL1R2, MMP8 | | | | | | | | | |
| RSAD2, OAS1, SIGLEC1, ISG15, SLC1A2, | 0.861 | 0.851 | 0.870 | 0.909 | 0.853 | 0.965 | 0.968 | 0.931 | 1 |
| IL1R2, RETN | | | | | | | | | |
| RSAD2, IFIT1, IFI44L, SIGLEC1, SLC1A2, | 0.861 | 0.852 | 0.869 | 0.908 | 0.853 | 0.963 | 0.968 | 0.934 | 1 |
| IL1R2, MMP8 | | | | | | | | | |
| RSAD2, IFIT1, SIGLEC1, IL1R2, OLAH, RETN, | 0.861 | 0.851 | 0.870 | 0.901 | 0.838 | 0.964 | 0.967 | 0.933 | 1 |
| MMP8 | | | | | | | | | |
| RSAD2, OAS1, IFIT1, IFI44L, SLC1A2, IL1R2, | 0.861 | 0.851 | 0.870 | 0.906 | 0.849 | 0.963 | 0.927 | 0.842 | 1 |
| FAM20A | | | | | | | | | |
| OAS1, IFI44L, SIGLEC1, SLC1A2, IL1R2, RETN, | 0.861 | 0.851 | 0.870 | 0.912 | 0.857 | 0.966 | 0.968 | 0.934 | 1 |
| MMP8 | | | | | | | | | |
| RSAD2, IFI44L, ISG15, SLC1A2, OLAH, | 0.861 | 0.850 | 0.871 | 0.909 | 0.850 | 0.968 | 0.926 | 0.852 | 1 |
| FAM20A, RETN | | | | | | | | | |
| RSAD2, IFIT1, IFI44L, SIGLEC1, ISG15, IL1R2, | 0.861 | 0.852 | 0.869 | 0.900 | 0.838 | 0.962 | 0.960 | 0.919 | 1 |
| MMP8 | | | | | | | | | |
| RSAD2, IFIT1, HERC6, SLC1A2, OLAH, | 0.860 | 0.850 | 0.871 | 0.910 | 0.85. | 0.967 | 0.932 | 0.850 | 1 |
| FAM20A, RETN | | | | | | | | | |
| RSAD2, IFI44L, SIGLEC1, ISG15, IL1R2, OLAH, | 0.860 | 0.851 | 0.870 | 0.900 | 0.839 | 0.962 | 0.959 | 0.917 | 1 |
| MMP8 | | | | | | | | | |
| RSAD2, OAS1, SIGLEC1, ISG15, HERC6, | 0.860 | 0.850 | 0.871 | 0.899 | 0.835 | 0.964 | 0.952 | 0.906 | 0.998 |
| OLAH, RETN | | | | | | | | | |
| OAS1, IFIT1, SIGLEC1, ISG15, IL1R2, OLAH, | 0.860 | 0.851 | 0.870 | 0.900 | 0.836 | 0.965 | 0.954 | 0.910 | 0.999 |
| RETN | | | | | | | | | |
| IFI27, OAS1, IFIT1, IFI44L, SLC1A2, FAM20A, | 0.860 | 0.850 | 0.871 | 0.901 | 0.837 | 0.966 | 0.932 | 0.850 | 1 |
| MMP8 | | | | | | | | | |
| RSAD2, IFIT1, IFI44L, SIGLEC1, ISG15, IL1R2, | 0.860 | 0.851 | 0.869 | 0.898 | 0.832 | 0.964 | 0.957 | 0.914 | 0.999 |
| OLAH | | | | | | | | | |
| RSAD2, IFI27, OAS1, IFIT1, ISG15, HERC6, | 0.860 | 0.851 | 0.869 | 0.901 | 0.843 | 0.958 | 0.901 | 0.823 | 0.979 |
| MMP8 | | | | | | | | | |
| RSAD2, IFIT1, SIGLEC1, ISG15, SLC1A2, | 0.860 | 0.852 | 0.869 | 0.908 | 0.853 | 0.963 | 0.965 | 0.929 | 1 |
| IL1R2, MMP8 | | | | | | | | | |
| OAS1, IFI44L, SIGLEC1, ISG15, HERC6, | 0.860 | 0.850 | 0.871 | 0.899 | 0.834 | 0.965 | 0.953 | 0.891 | 1 |
| FAM20A, MMP8 | | | | | | | | | |
| IFIT1, ISG15, IL1R2, OLAH, FAM20A, RETN, | 0.860 | 0.850 | 0.870 | 0.906 | 0.845 | 0.967 | 0.948 | 0.882 | 1 |
| MMP8 | | | | | | | | | |
| OAS1, IFIT1, IFI44L, SIGLEC1, IL1R2, OLAH, | 0.860 | 0.851 | 0.870 | 0.900 | 0.836 | 0.965 | 0.955 | 0.912 | 0.999 |
| RETN | | | | | | | | | |
| RSAD2, IFIT1, SIGLEC1, ISG15, SLC1A2, | 0.860 | 0.851 | 0.869 | 0.908 | 0.852 | 0.964 | 0.965 | 0.928 | 1 |
| IL1R2, RETN | | | | | | | | | |
| RSAD2, IFI44L, SIGLEC1, IL1R2, OLAH, RETN, | 0.860 | 0.851 | 0.870 | 0.901 | 0.838 | 0.965 | 0.967 | 0.932 | 1 |
| MMP8 | | | | | | | | | |
| OAS1, IFI44L, SIGLEC1, ISG15, SLC1A2, IL1R2, | 0.860 | 0.851 | 0.869 | 0.910 | 0.855 | 0.965 | 0.965 | 0.927 | 1 |
| RETN | | | | | | | | | |
| RSAD2, SIGLEC1, ISG15, SLC1A2, IL1R2, | 0.860 | 0.851 | 0.869 | 0.913 | 0.859 | 0.966 | 0.972 | 0.941 | 1 |
| RETN, MMP8 | | | | | | | | | |
| RSAD2, OAS1, SIGLEC1, ISG15, HERC6, RETN, | 0.860 | 0.850 | 0.870 | 0.899 | 0.832 | 0.965 | 0.951 | 0.904 | 0.998 |
| MMP8 | | | | | | | | | |
| RSAD2, IFIT1, IFI44L, SIGLEC1, ISG15, | 0.860 | 0.851 | 0.869 | 0.905 | 0.848 | 0.962 | 0.968 | 0.935 | 1 |
| SLC1A2, IL1R2 | | | | | | | | | |
| OAS1, IFIT1, IFI44L, SIGLEC1, ISG15, IL1R2, | 0.860 | 0.851 | 0.870 | 0.899 | 0.833 | 0.964 | 0.959 | 0.918 | 0.999 |
| RETN | | | | | | | | | |
| RSAD2, OAS1, IFI44L, SIGLEC1, SLC1A2, | 0.860 | 0.850 | 0.870 | 0.905 | 0.843 | 0.967 | 0.945 | 0.873 | 1 |
| FAM20A, RETN | | | | | | | | | |
| RSAD2, OAS1, SIGLEC1, ISG15, SLC1A2, | 0.860 | 0.851 | 0.869 | 0.909 | 0.855 | 0.963 | 0.968 | 0.934 | 1 |
| IL1R2, MMP8 | | | | | | | | | |
| RSAD2, OAS1, IFIT1, IFI44L, SIGLEC1, | 0.860 | 0.851 | 0.869 | 0.905 | 0.849 | 0.962 | 0.970 | 0.935 | 1 |
| SLC1A2, IL1R2 | | | | | | | | | |
| RSAD2, OAS1, IFI44L, SIGLEC1, SLC1A2, | 0.860 | 0.851 | 0.869 | 0.906 | 0.849 | 0.963 | 0.967 | 0.929 | 1 |
| IL1R2, RETN | | | | | | | | | |
| IFIT1, IFI44L, ISG15, IL1R2, FAM20A, RETN, | 0.860 | 0.850 | 0.870 | 0.903 | 0.839 | 0.966 | 0.946 | 0.873 | 1 |
| MMP8 | | | | | | | | | |
| RSAD2, OAS1, IFIT1, ISG15, IL1R2, FAM20A, | 0.860 | 0.850 | 0.870 | 0.902 | 0.838 | 0.966 | 0.943 | 0.869 | 1 |
| RETN | | | | | | | | | |
| RSAD2, IFIT1, IFI44L, SIGLEC1, SLC1A2, | 0.860 | 0.851 | 0.869 | 0.907 | 0.850 | 0.963 | 0.965 | 0.928 | 1 |
| IL1R2, RETN | | | | | | | | | |
| OAS1, IFIT1, IFI44L, SIGLEC1, OLAH, RETN, | 0.860 | 0.850 | 0.870 | 0.897 | 0.830 | 0.964 | 0.955 | 0.912 | 0.999 |
| MMP8 | | | | | | | | | |
| RSAD2, SIGLEC1, ISG15, HERC6, OLAH, | 0.860 | 0.850 | 0.870 | 0.900 | 0.835 | 0.966 | 0.958 | 0.916 | 0.999 |
| RETN, MMP8 | | | | | | | | | |
| RSAD2, IFI44L, SIGLEC1, ISG15, SLC1A2, | 0.860 | 0.851 | 0.869 | 0.907 | 0.850 | 0.963 | 0.970 | 0.937 | 1 |
| IL1R2, RETN | | | | | | | | | |
| OAS1, IFI44L, SIGLEC1, ISG15, IL1R2, RETN, | 0.860 | 0.850 | 0.870 | 0.901 | 0.837 | 0.965 | 0.962 | 0.923 | 1 |
| MMP8 | | | | | | | | | |
| RSAD2, IFIT1, IFI44L, ISG15, SLC1A2, IL1R2, | 0.860 | 0.851 | 0.869 | 0.906 | 0.850 | 0.962 | 0.927 | 0.840 | 1 |
| FAM20A | | | | | | | | | |
| RSAD2, IFI27, OAS1, IFI44L, ISG15, SLC1A2, | 0.860 | 0.849 | 0.871 | 0.902 | 0.838 | 0.966 | 0.935 | 0.855 | 1 |
| FAM20A | | | | | | | | | |
| RSAD2, IFI44L, ISG15, HERC6, FAM20A, | 0.860 | 0.850 | 0.870 | 0.908 | 0.846 | 0.969 | 0.934 | 0.858 | 1 |
| RETN, MMP8 | | | | | | | | | |
| RSAD2, OAS1, IFI44L, SIGLEC1, IL1R2, OLAH, | 0.860 | 0.850 | 0.870 | 0.895 | 0.829 | 0.962 | 0.962 | 0.922 | 1 |
| RETN | | | | | | | | | |
| RSAD2, OAS1, IFI44L, SIGLEC1, IL1R2, RETN, | 0.860 | 0.850 | 0.870 | 0.900 | 0.836 | 0.964 | 0.970 | 0.937 | 1 |
| MMP8 | | | | | | | | | |
| OAS1, SIGLEC1, ISG15, IL1R2, OLAH, RETN, | 0.860 | 0.850 | 0.870 | 0.900 | 0.837 | 0.964 | 0.970 | 0.936 | 1 |
| MMP8 | | | | | | | | | |
| RSAD2, IFIT1, IFI44L, SIGLEC1, IL1R2, OLAH, | 0.860 | 0.851 | 0.869 | 0.898 | 0.831 | 0.964 | 0.961 | 0.921 | 1 |
| RETN | | | | | | | | | |
| OAS1, IFI44L, SIGLEC1, ISG15, HERC6, OLAH, | 0.860 | 0.850 | 0.870 | 0.898 | 0.833 | 0.964 | 0.952 | 0.906 | 0.998 |
| RETN | | | | | | | | | |
| RSAD2, IFIT1, IFI44L, SIGLEC1, HERC6, | 0.860 | 0.850 | 0.869 | 0.899 | 0.836 | 0.961 | 0.936 | 0.881 | 0.991 |
| OLAH, MMP8 | | | | | | | | | |
| IFIT1, IFI44L, ISG15, HERC6, FAM20A, RETN, | 0.860 | 0.850 | 0.870 | 0.905 | 0.840 | 0.969 | 0.937 | 0.860 | 1 |
| MMP8 | | | | | | | | | |
| IFI27, OAS1, IFI44L, ISG15, SLC1A2, FAM20A, | 0.860 | 0.848 | 0.871 | 0.901 | 0.836 | 0.966 | 0.937 | 0.856 | 1 |
| MMP8 | | | | | | | | | |
| OAS1, IFI44L, SIGLEC1, ISG15, HERC6, | 0.860 | 0.850 | 0.870 | 0.905 | 0.844 | 0.965 | 0.952 | 0.902 | 1 |
| SLC1A2, RETN | | | | | | | | | |
| OAS1, IFI44L, SIGLEC1, ISG15, SLC1A2, | 0.860 | 0.849 | 0.870 | 0.903 | 0.840 | 0.965 | 0.948 | 0.877 | 1 |
| FAM20A, MMP8 | | | | | | | | | |
| RSAD2, OAS1, IFI44L, ISG15, SLC1A2, IL1R2, | 0.860 | 0.850 | 0.870 | 0.908 | 0.851 | 0.964 | 0.926 | 0.839 | 1 |
| FAM20A | | | | | | | | | |
| RSAD2, IFI27, OAS1, IFIT1, HERC6, SLC1A2, | 0.860 | 0.850 | 0.869 | 0.907 | 0.852 | 0.962 | 0.898 | 0.815 | 0.981 |
| MMP8 | | | | | | | | | |
| RSAD2, OAS1, IFI44L, SIGLEC1, SLC1A2, | 0.860 | 0.851 | 0.869 | 0.907 | 0.852 | 0.962 | 0.972 | 0.940 | 1 |
| IL1R2, MMP8 | | | | | | | | | |
| RSAD2, OAS1, IFI44L, SIGLEC1, ISG15, | 0.860 | 0.850 | 0.869 | 0.905 | 0.848 | 0.962 | 0.967 | 0.930 | 1 |
| SLC1A2, IL1R2 | | | | | | | | | |
| RSAD2, OAS1, IFIT1, IFI44L, SIGLEC1, HERC6, | 0.860 | 0.850 | 0.869 | 0.900 | 0.838 | 0.961 | 0.939 | 0.886 | 0.992 |
| OLAH | | | | | | | | | |
| RSAD2, OAS1, IFIT1, SLC1A2, IL1R2, FAM20A, | 0.860 | 0.850 | 0.869 | 0.907 | 0.851 | 0.964 | 0.936 | 0.855 | 1 |
| RETN | | | | | | | | | |
| IFI27, OAS1, IFIT1, ISG15, HERC6, SLC1A2, | 0.860 | 0.849 | 0.870 | 0.904 | 0.845 | 0.963 | 0.930 | 0.847 | 1 |
| FAM20A | | | | | | | | | |
| RSAD2, IFI27, OAS1, ISG15, HERC6, SLC1A2, | 0.860 | 0.850 | 0.869 | 0.906 | 0.851 | 0.961 | 0.901 | 0.820 | 0.982 |
| MMP8 | | | | | | | | | |
| RSAD2, IFI44L, SIGLEC1, SLC1A2, IL1R2, | 0.860 | 0.850 | 0.869 | 0.910 | 0.855 | 0.965 | 0.971 | 0.939 | 1 |
| RETN, MMP8 | | | | | | | | | |
| RSAD2, IFI27, OAS1, IFI44L, ISG15, SLC1A2, | 0.860 | 0.850 | 0.869 | 0.904 | 0.842 | 0.967 | 0.943 | 0.874 | 1 |
| RETN | | | | | | | | | |
| RSAD2, IFI44L, SIGLEC1, ISG15, IL1R2, RETN, | 0.860 | 0.850 | 0.869 | 0.900 | 0.835 | 0.964 | 0.967 | 0.933 | 1 |
| MMP8 | | | | | | | | | |
| RSAD2, OAS1, IFI44L, SIGLEC1, ISG15, IL1R2, | 0.859 | 0.850 | 0.869 | 0.900 | 0.838 | 0.961 | 0.960 | 0.919 | 1 |
| MMP8 | | | | | | | | | |
| RSAD2, OAS1, ISG15, IL1R2, FAM20A, RETN, | 0.859 | 0.849 | 0.870 | 0.905 | 0.843 | 0.968 | 0.939 | 0.860 | 1 |
| MMP8 | | | | | | | | | |
| RSAD2, IFI27, OAS1, IFIT1, IFI44L, RETN, | 0.859 | 0.849 | 0.870 | 0.905 | 0.842 | 0.968 | 0.949 | 0.884 | 1 |
| MMP8 | | | | | | | | | |
| OAS1, IFI44L, ISG15, IL1R2, OLAH, FAM20A, | 0.859 | 0.849 | 0.870 | 0.900 | 0.834 | 0.965 | 0.943 | 0.874 | 1 |
| RETN | | | | | | | | | |
| OAS1, IFIT1, SLC1A2, OLAH, FAM20A, RETN, | 0.859 | 0.849 | 0.870 | 0.906 | 0.845 | 0.967 | 0.939 | 0.866 | 1 |
| MMP8 | | | | | | | | | |
| OAS1, IFI44L, SLC1A2, IL1R2, FAM20A, RETN, | 0.859 | 0.850 | 0.869 | 0.912 | 0.859 | 0.965 | 0.925 | 0.832 | 1 |
| MMP8 | | | | | | | | | |
| RSAD2, IFI44L, ISG15, HERC6, SLC1A2, | 0.859 | 0.849 | 0.870 | 0.905 | 0.844 | 0.966 | 0.930 | 0.851 | 1 |
| FAM20A, RETN | | | | | | | | | |
| RSAD2, IFI27, OAS1, IFIT1, ISG15, RETN, | 0.859 | 0.849 | 0.870 | 0.904 | 0.841 | 0.968 | 0.950 | 0.886 | 1 |
| MMP8 | | | | | | | | | |
| OAS1, IFI44L, SIGLEC1, ISG15, SLC1A2, IL1R2, | 0.859 | 0.851 | 0.868 | 0.908 | 0.853 | 0.962 | 0.965 | 0.928 | 1 |
| MMP8 | | | | | | | | | |
| RSAD2, SIGLEC1, ISG15, IL1R2, OLAH, RETN, | 0.859 | 0.849 | 0.870 | 0.901 | 0.838 | 0.964 | 0.971 | 0.938 | 1 |
| MMP8 | | | | | | | | | |
| RSAD2, OAS1, IFI44L, SIGLEC1, ISG15, | 0.859 | 0.849 | 0.869 | 0.898 | 0.832 | 0.964 | 0.949 | 0.901 | 0.997 |
| HERC6, RETN | | | | | | | | | |
| RSAD2, IFI27, OAS1, IFI44L, ISG15, RETN, | 0.859 | 0.849 | 0.869 | 0.905 | 0.842 | 0.969 | 0.950 | 0.887 | 1 |
| MMP8 | | | | | | | | | |
| RSAD2, IFIT1, ISG15, HERC6, OLAH, FAM20A, | 0.859 | 0.849 | 0.869 | 0.901 | 0.840 | 0.962 | 0.939 | 0.869 | 1 |
| MMP8 | | | | | | | | | |
| OAS1, IFIT1, IFI44L, IL1R2, OLAH, FAM20A, | 0.859 | 0.850 | 0.869 | 0.900 | 0.834 | 0.966 | 0.942 | 0.872 | 1 |
| RETN | | | | | | | | | |
| IFI27, OAS1, IFIT1, ISG15, SLC1A2, RETN, | 0.859 | 0.849 | 0.869 | 0.900 | 0.836 | 0.965 | 0.941 | 0.870 | 1 |
| MMP8 | | | | | | | | | |
| RSAD2, IFI44L, ISG15, SLC1A2, IL1R2, | 0.859 | 0.849 | 0.869 | 0.908 | 0.853 | 0.963 | 0.928 | 0.842 | 1 |
| FAM20A, RETN | | | | | | | | | |
| RSAD2, OAS1, SIGLEC1, ISG15, IL1R2, RETN, | 0.859 | 0.849 | 0.870 | 0.900 | 0.836 | 0.963 | 0.970 | 0.936 | 1 |
| MMP8 | | | | | | | | | |
| RSAD2, OAS1, ISG15, SLC1A2, IL1R2, | 0.859 | 0.850 | 0.869 | 0.910 | 0.856 | 0.963 | 0.927 | 0.835 | 1 |
| FAM20A, MMP8 | | | | | | | | | |
| RSAD2, IFIT1, IFI44L, SIGLEC1, ISG15, IL1R2, | 0.859 | 0.850 | 0.868 | 0.897 | 0.831 | 0.964 | 0.961 | 0.922 | 1.000 |
| RETN | | | | | | | | | |
| RSAD2, IFI27, OAS1, IFI44L, HERC6, SLC1A2, | 0.859 | 0.850 | 0.869 | 0.907 | 0.851 | 0.962 | 0.903 | 0.822 | 0.984 |
| MMP8 | | | | | | | | | |
| RSAD2, IFIT1, ISG15, SLC1A2, OLAH, | 0.859 | 0.850 | 0.869 | 0.905 | 0.845 | 0.965 | 0.939 | 0.869 | 1 |
| FAM20A, MMP8 | | | | | | | | | |
| RSAD2, IFIT1, IL1R2, OLAH, FAM20A, RETN, | 0.859 | 0.849 | 0.869 | 0.904 | 0.842 | 0.967 | 0.948 | 0.881 | 1 |
| MMP8 | | | | | | | | | |
| RSAD2, OAS1, IFI44L, IL1R2, FAM20A, RETN, | 0.859 | 0.849 | 0.870 | 0.907 | 0.846 | 0.968 | 0.941 | 0.865 | 1 |
| MMP8 | | | | | | | | | |
| RSAD2, IFI27, OAS1, IFIT1, ISG15, SLC1A2, | 0.859 | 0.849 | 0.869 | 0.901 | 0.836 | 0.966 | 0.942 | 0.869 | 1 |
| RETN | | | | | | | | | |
| RSAD2, IFI27, IFIT1, SIGLEC1, ISG15, SLC1A2, | 0.859 | 0.850 | 0.868 | 0.904 | 0.845 | 0.963 | 0.925 | 0.855 | 0.995 |
| MMP8 | | | | | | | | | |
| RSAD2, IFI27, IFIT1, ISG15, HERC6, SLC1A2, | 0.859 | 0.848 | 0.870 | 0.908 | 0.850 | 0.966 | 0.930 | 0.847 | 1 |
| FAM20A | | | | | | | | | |
| RSAD2, IFIT1, ISG15, SLC1A2, IL1R2, | 0.859 | 0.850 | 0.868 | 0.907 | 0.850 | 0.964 | 0.935 | 0.853 | 1 |
| FAM20A, RETN | | | | | | | | | |
| IFIT1, IFI44L, SIGLEC1, ISG15, IL1R2, OLAH, | 0.859 | 0.850 | 0.868 | 0.900 | 0.837 | 0.964 | 0.961 | 0.919 | 1 |
| RETN | | | | | | | | | |
| RSAD2, IFI44L, SIGLEC1, ISG15, SLC1A2, | 0.859 | 0.850 | 0.868 | 0.906 | 0.851 | 0.962 | 0.968 | 0.935 | 1 |
| IL1R2, MMP8 | | | | | | | | | |
| RSAD2, OAS1, IFIT1, SIGLEC1, ISG15, HERC6, | 0.859 | 0.849 | 0.869 | 0.900 | 0.839 | 0.962 | 0.941 | 0.890 | 0.993 |
| OLAH | | | | | | | | | |
| RSAD2, OAS1, SLC1A2, OLAH, FAM20A, | 0.859 | 0.849 | 0.869 | 0.908 | 0.849 | 0.967 | 0.938 | 0.864 | 1 |
| RETN, MMP8 | | | | | | | | | |
| RSAD2, IFI44L, SLC1A2, IL1R2, FAM20A, | 0.859 | 0.849 | 0.868 | 0.914 | 0.862 | 0.966 | 0.926 | 0.835 | 1 |
| RETN, MMP8 | | | | | | | | | |
| RSAD2, OAS1, IFI44L, SIGLEC1, ISG15, IL1R2, | 0.859 | 0.849 | 0.868 | 0.893 | 0.826 | 0.961 | 0.962 | 0.923 | 1 |
| RETN | | | | | | | | | |
| RSAD2, OAS1, SLC1A2, IL1R2, FAM20A, | 0.859 | 0.850 | 0.868 | 0.913 | 0.859 | 0.966 | 0.928 | 0.837 | 1 |
| RETN, MMP8 | | | | | | | | | |
| RSAD2, OAS1, IFI44L, SLC1A2, IL1R2, | 0.859 | 0.849 | 0.869 | 0.907 | 0.851 | 0.963 | 0.923 | 0.834 | 1 |
| FAM20A, RETN | | | | | | | | | |
| OAS1, IFIT1, IFI44L, SLC1A2, IL1R2, FAM20A, | 0.859 | 0.849 | 0.869 | 0.908 | 0.852 | 0.963 | 0.924 | 0.835 | 1 |
| RETN | | | | | | | | | |
| IFIT1, IFI44L, ISG15, HERC6, SLC1A2, | 0.859 | 0.849 | 0.869 | 0.907 | 0.848 | 0.967 | 0.934 | 0.853 | 1 |
| FAM20A, RETN | | | | | | | | | |
| RSAD2, IFI27, IFIT1, IFI44L, ISG15, SLC1A2, | 0.859 | 0.848 | 0.869 | 0.901 | 0.836 | 0.966 | 0.939 | 0.860 | 1 |
| FAM20A | | | | | | | | | |
| IFI27, OAS1, IFI44L, ISG15, SLC1A2, RETN, | 0.859 | 0.849 | 0.869 | 0.904 | 0.842 | 0.966 | 0.943 | 0.877 | 1 |
| MMP8 | | | | | | | | | |
| IFI27, OAS1, IFIT1, IFI44L, SLC1A2, RETN, | 0.859 | 0.849 | 0.869 | 0.903 | 0.839 | 0.966 | 0.940 | 0.867 | 1 |
| MMP8 | | | | | | | | | |
| RSAD2, IFIT1, SIGLEC1, ISG15, HERC6, OLAH, | 0.859 | 0.849 | 0.869 | 0.900 | 0.838 | 0.962 | 0.943 | 0.893 | 0.994 |
| MMP8 | | | | | | | | | |
| RSAD2, IFI27, OAS1, IFIT1, IFI44L, SLC1A2, | 0.859 | 0.848 | 0.870 | 0.903 | 0.839 | 0.967 | 0.935 | 0.855 | 1 |
| FAM20A | | | | | | | | | |
| OAS1, IFI44L, ISG15, SLC1A2, IL1R2, FAM20A, | 0.859 | 0.848 | 0.869 | 0.908 | 0.852 | 0.963 | 0.925 | 0.837 | 1 |
| RETN | | | | | | | | | |
| RSAD2, OAS1, IFIT1, IFI44L, IL1R2, FAM20A, | 0.859 | 0.849 | 0.869 | 0.901 | 0.837 | 0.966 | 0.939 | 0.864 | 1 |
| RETN | | | | | | | | | |
| RSAD2, IFI27, OAS1, IFIT1, IFI44L, HERC6, | 0.859 | 0.849 | 0.868 | 0.900 | 0.841 | 0.958 | 0.899 | 0.819 | 0.978 |
| MMP8 | | | | | | | | | |
| RSAD2, IFIT1, SIGLEC1, ISG15, IL1R2, OLAH, | 0.859 | 0.849 | 0.868 | 0.904 | 0.846 | 0.963 | 0.965 | 0.929 | 1 |
| MMP8 | | | | | | | | | |
| IFIT1, ISG15, SLC1A2, OLAH, FAM20A, RETN, | 0.859 | 0.848 | 0.869 | 0.906 | 0.847 | 0.966 | 0.942 | 0.875 | 1 |
| MMP8 | | | | | | | | | |
| OAS1, IFIT1, ISG15, HERC6, SLC1A2, FAM20A, | 0.859 | 0.848 | 0.869 | 0.902 | 0.840 | 0.964 | 0.936 | 0.855 | 1 |
| RETN | | | | | | | | | |
| RSAD2, OAS1, ISG15, SLC1A2, OLAH, | 0.859 | 0.848 | 0.869 | 0.910 | 0.851 | 0.968 | 0.934 | 0.859 | 1 |
| FAM20A, RETN | | | | | | | | | |
| OAS1, IFIT1, ISG15, SLC1A2, OLAH, FAM20A, | 0.859 | 0.848 | 0.869 | 0.908 | 0.849 | 0.967 | 0.936 | 0.862 | 1 |
| RETN | | | | | | | | | |
| RSAD2, OAS1, IFIT1, ISG15, SLC1A2, OLAH, | 0.859 | 0.849 | 0.869 | 0.908 | 0.849 | 0.967 | 0.935 | 0.861 | 1 |
| FAM20A | | | | | | | | | |
| OAS1, ISG15, SLC1A2, IL1R2, FAM20A, RETN, | 0.859 | 0.849 | 0.868 | 0.913 | 0.859 | 0.967 | 0.929 | 0.841 | 1 |
| MMP8 | | | | | | | | | |
| OAS1, IFI44L, ISG15, HERC6, OLAH, FAM20A, | 0.859 | 0.848 | 0.869 | 0.898 | 0.834 | 0.962 | 0.947 | 0.885 | 1 |
| MMP8 | | | | | | | | | |
| OAS1, IFI44L, SIGLEC1, ISG15, IL1R2, OLAH, | 0.859 | 0.849 | 0.868 | 0.899 | 0.834 | 0.964 | 0.957 | 0.914 | 0.999 |
| RETN | | | | | | | | | |
| RSAD2, OAS1, ISG15, HERC6, FAM20A, RETN, | 0.859 | 0.848 | 0.869 | 0.904 | 0.842 | 0.967 | 0.936 | 0.859 | 1 |
| MMP8 | | | | | | | | | |
| RSAD2, IFIT1, SIGLEC1, ISG15, HERC6, RETN, | 0.859 | 0.849 | 0.868 | 0.898 | 0.832 | 0.964 | 0.930 | 0.873 | 0.988 |
| MMP8 | | | | | | | | | |
| RSAD2, IFIT1, SLC1A2, OLAH, FAM20A, | 0.858 | 0.849 | 0.868 | 0.906 | 0.846 | 0.967 | 0.941 | 0.871 | 1 |
| RETN, MMP8 | | | | | | | | | |
| OAS1, IFIT1, IFI44L, HERC6, SLC1A2, OLAH, | 0.858 | 0.848 | 0.869 | 0.907 | 0.849 | 0.966 | 0.928 | 0.865 | 0.992 |
| RETN | | | | | | | | | |
| RSAD2, IFI27, OAS1, IFIT1, IFI44L, SLC1A2, | 0.858 | 0.849 | 0.868 | 0.903 | 0.839 | 0.968 | 0.943 | 0.872 | 1 |
| RETN | | | | | | | | | |
| OAS1, IFI44L, ISG15, HERC6, SLC1A2, | 0.858 | 0.848 | 0.869 | 0.904 | 0.844 | 0.965 | 0.934 | 0.854 | 1 |
| FAM20A, RETN | | | | | | | | | |
| IFI27, OAS1, IFI44L, ISG15, HERC6, SLC1A2, | 0.858 | 0.849 | 0.868 | 0.906 | 0.852 | 0.960 | 0.900 | 0.821 | 0.979 |
| MMP8 | | | | | | | | | |
| IFI27, OAS1, IFIT1, ISG15, HERC6, SLC1A2, | 0.858 | 0.849 | 0.868 | 0.904 | 0.849 | 0.959 | 0.902 | 0.823 | 0.981 |
| MMP8 | | | | | | | | | |
| RSAD2, IFIT1, SIGLEC1, ISG15, HERC6, | 0.858 | 0.849 | 0.868 | 0.904 | 0.844 | 0.963 | 0.934 | 0.877 | 0.990 |
| SLC1A2, RETN | | | | | | | | | |
| RSAD2, ISG15, IL1R2, OLAH, FAM20A, RETN, | 0.858 | 0.848 | 0.868 | 0.902 | 0.839 | 0.965 | 0.941 | 0.868 | 1 |
| MMP8 | | | | | | | | | |
| OAS1, IFI44L, HERC6, SLC1A2, OLAH, RETN, | 0.858 | 0.848 | 0.869 | 0.907 | 0.848 | 0.966 | 0.939 | 0.884 | 0.994 |
| MMP8 | | | | | | | | | |
| IFI27, OAS1, IFIT1, IFI44L, HERC6, SLC1A2, | 0.858 | 0.849 | 0.868 | 0.904 | 0.849 | 0.959 | 0.908 | 0.832 | 0.983 |
| MMP8 | | | | | | | | | |
| RSAD2, IFIT1, SIGLEC1, ISG15, IL1R2, RETN, | 0.858 | 0.849 | 0.868 | 0.901 | 0.838 | 0.965 | 0.972 | 0.940 | 1 |
| MMP8 | | | | | | | | | |
| OAS1, IFIT1, SIGLEC1, ISG15, OLAH, RETN, | 0.858 | 0.848 | 0.868 | 0.897 | 0.830 | 0.964 | 0.952 | 0.906 | 0.998 |
| MMP8 | | | | | | | | | |
| OAS1, ISG15, SLC1A2, OLAH, FAM20A, RETN, | 0.858 | 0.848 | 0.868 | 0.908 | 0.848 | 0.967 | 0.937 | 0.863 | 1 |
| MMP8 | | | | | | | | | |
| RSAD2, OAS1, IFIT1, SIGLEC1, ISG15, IL1R2, | 0.858 | 0.849 | 0.868 | 0.897 | 0.831 | 0.963 | 0.962 | 0.921 | 1 |
| RETN | | | | | | | | | |
| RSAD2, IFIT1, IFI44L, ISG15, IL1R2, FAM20A, | 0.858 | 0.848 | 0.868 | 0.904 | 0.842 | 0.966 | 0.942 | 0.867 | 1 |
| MMP8 | | | | | | | | | |
| RSAD2, OAS1, ISG15, SLC1A2, IL1R2, | 0.858 | 0.848 | 0.868 | 0.905 | 0.849 | 0.962 | 0.930 | 0.844 | 1 |
| FAM20A, RETN | | | | | | | | | |
| RSAD2, ISG15, SLC1A2, OLAH, FAM20A, | 0.858 | 0.848 | 0.868 | 0.907 | 0.847 | 0.966 | 0.941 | 0.871 | 1 |
| RETN, MMP8 | | | | | | | | | |
| RSAD2, OAS1, IFIT1, IFI44L, OLAH, FAM20A, | 0.858 | 0.848 | 0.868 | 0.898 | 0.831 | 0.966 | 0.946 | 0.886 | 1 |
| MMP8 | | | | | | | | | |
| IFI44L, HERC6, SLC1A2, IL1R2, OLAH, RETN, | 0.858 | 0.848 | 0.868 | 0.908 | 0.849 | 0.966 | 0.947 | 0.897 | 0.997 |
| MMP8 | | | | | | | | | |
| IFIT1, IFI44L, HERC6, SLC1A2, OLAH, RETN, | 0.858 | 0.848 | 0.868 | 0.906 | 0.847 | 0.965 | 0.940 | 0.887 | 0.993 |
| MMP8 | | | | | | | | | |
| RSAD2, IFIT1, IFI44L, SLC1A2, IL1R2, | 0.858 | 0.849 | 0.867 | 0.907 | 0.853 | 0.962 | 0.924 | 0.835 | 1 |
| FAM20A, RETN | | | | | | | | | |
| RSAD2, OAS1, SIGLEC1, ISG15, IL1R2, OLAH, | 0.858 | 0.848 | 0.868 | 0.898 | 0.832 | 0.963 | 0.963 | 0.923 | 1 |
| RETN | | | | | | | | | |
| RSAD2, IFI27, OAS1, IFIT1, SLC1A2, RETN, | 0.858 | 0.848 | 0.868 | 0.902 | 0.838 | 0.966 | 0.940 | 0.866 | 1 |
| MMP8 | | | | | | | | | |
| RSAD2, OAS1, IFI44L, ISG15, IL1R2, FAM20A, | 0.858 | 0.848 | 0.868 | 0.902 | 0.838 | 0.965 | 0.939 | 0.863 | 1 |
| RETN | | | | | | | | | |
| IFIT1, IFI44L, ISG15, IL1R2, OLAH, FAM20A, | 0.858 | 0.848 | 0.868 | 0.901 | 0.837 | 0.965 | 0.949 | 0.890 | 1 |
| RETN | | | | | | | | | |
| RSAD2, IFI27, IFIT1, IFI44L, SIGLEC1, SLC1A2, | 0.858 | 0.849 | 0.867 | 0.902 | 0.842 | 0.961 | 0.927 | 0.858 | 0.996 |
| MMP8 | | | | | | | | | |
| OAS1, IFIT1, IFI44L, ISG15, IL1R2, FAM20A, | 0.858 | 0.848 | 0.868 | 0.900 | 0.836 | 0.965 | 0.941 | 0.866 | 1 |
| RETN | | | | | | | | | |
| RSAD2, OAS1, ISG15, HERC6, SLC1A2, | 0.858 | 0.847 | 0.868 | 0.902 | 0.841 | 0.964 | 0.936 | 0.855 | 1 |
| FAM20A, RETN | | | | | | | | | |
| RSAD2, OAS1, SIGLEC1, HERC6, SLC1A2, | 0.858 | 0.848 | 0.868 | 0.903 | 0.840 | 0.965 | 0.950 | 0.900 | 1.000 |
| RETN, MMP8 | | | | | | | | | |
| RSAD2, IFI27, OAS1, IFI44L, SLC1A2, RETN, | 0.858 | 0.848 | 0.868 | 0.905 | 0.842 | 0.968 | 0.933 | 0.858 | 1 |
| MMP8 | | | | | | | | | |
| RSAD2, OAS1, IFIT1, SIGLEC1, ISG15, IL1R2, | 0.858 | 0.848 | 0.867 | 0.901 | 0.842 | 0.961 | 0.966 | 0.931 | 1 |
| MMP8 | | | | | | | | | |
| RSAD2, IFI44L, SIGLEC1, HERC6, OLAH, | 0.858 | 0.848 | 0.868 | 0.899 | 0.832 | 0.966 | 0.958 | 0.916 | 0.999 |
| RETN, MMP8 | | | | | | | | | |
| OAS1, IFIT1, ISG15, HERC6, IL1R2, RETN, | 0.858 | 0.848 | 0.868 | 0.897 | 0.832 | 0.961 | 0.960 | 0.918 | 1 |
| MMP8 | | | | | | | | | |
| RSAD2, OAS1, IFIT1, ISG15, HERC6, IL1R2, | 0.858 | 0.848 | 0.868 | 0.896 | 0.833 | 0.960 | 0.948 | 0.898 | 0.998 |
| RETN | | | | | | | | | |
| RSAD2, IFIT1, SIGLEC1, ISG15, IL1R2, OLAH, | 0.858 | 0.848 | 0.867 | 0.898 | 0.831 | 0.964 | 0.960 | 0.918 | 1 |
| RETN | | | | | | | | | |
| RSAD2, IFI44L, SIGLEC1, ISG15, IL1R2, OLAH, | 0.858 | 0.848 | 0.867 | 0.896 | 0.830 | 0.963 | 0.960 | 0.918 | 1 |
| RETN | | | | | | | | | |
| IFI27, IFIT1, IFI44L, ISG15, SLC1A2, RETN, | 0.858 | 0.847 | 0.868 | 0.902 | 0.839 | 0.965 | 0.942 | 0.875 | 1 |
| MMP8 | | | | | | | | | |
| ISG15, SLC1A2, IL1R2, OLAH, FAM20A, RETN, | 0.858 | 0.848 | 0.867 | 0.911 | 0.857 | 0.964 | 0.923 | 0.842 | 1 |
| MMP8 | | | | | | | | | |
| RSAD2, OAS1, IFIT1, SIGLEC1, OLAH, RETN, | 0.858 | 0.847 | 0.868 | 0.896 | 0.829 | 0.964 | 0.954 | 0.910 | 0.999 |
| MMP8 | | | | | | | | | |
| RSAD2, IFIT1, IFI44L, ISG15, IL1R2, OLAH, | 0.858 | 0.848 | 0.867 | 0.900 | 0.835 | 0.965 | 0.952 | 0.892 | 1 |
| FAM20A | | | | | | | | | |
| RSAD2, IFI27, IFI44L, SIGLEC1, ISG15, | 0.858 | 0.848 | 0.867 | 0.901 | 0.842 | 0.961 | 0.928 | 0.859 | 0.998 |
| SLC1A2, MMP8 | | | | | | | | | |
| OAS1, IFI44L, ISG15, HERC6, SLC1A2, OLAH, | 0.858 | 0.847 | 0.868 | 0.909 | 0.852 | 0.966 | 0.940 | 0.882 | 0.998 |
| RETN | | | | | | | | | |
| RSAD2, IFIT1, IFI44L, SIGLEC1, HERC6, | 0.858 | 0.849 | 0.866 | 0.903 | 0.843 | 0.963 | 0.923 | 0.862 | 0.984 |
| SLC1A2, RETN | | | | | | | | | |
| RSAD2, IFI27, OAS1, ISG15, SLC1A2, RETN, | 0.858 | 0.847 | 0.868 | 0.901 | 0.837 | 0.965 | 0.932 | 0.853 | 1 |
| MMP8 | | | | | | | | | |
| RSAD2, SIGLEC1, ISG15, HERC6, SLC1A2, | 0.857 | 0.848 | 0.867 | 0.906 | 0.846 | 0.966 | 0.946 | 0.895 | 0.996 |
| RETN, MMP8 | | | | | | | | | |
| RSAD2, OAS1, IFI44L, SIGLEC1, HERC6, | 0.857 | 0.848 | 0.867 | 0.896 | 0.829 | 0.963 | 0.949 | 0.901 | 0.996 |
| OLAH, RETN | | | | | | | | | |
| IFI27, OAS1, IFIT1, SIGLEC1, ISG15, HERC6, | 0.857 | 0.848 | 0.867 | 0.903 | 0.848 | 0.958 | 0.945 | 0.892 | 0.997 |
| SLC1A2 | | | | | | | | | |
| RSAD2, OAS1, IFIT1, SLC1A2, OLAH, | 0.857 | 0.847 | 0.868 | 0.908 | 0.849 | 0.968 | 0.934 | 0.859 | 1 |
| FAM20A, RETN | | | | | | | | | |
| RSAD2, IFIT1, IFI44L, SIGLEC1, ISG15, HERC6, | 0.857 | 0.848 | 0.867 | 0.900 | 0.839 | 0.962 | 0.940 | 0.888 | 0.992 |
| OLAH | | | | | | | | | |
| RSAD2, IFI27, IFIT1, IFI44L, ISG15, RETN, | 0.857 | 0.847 | 0.868 | 0.903 | 0.839 | 0.967 | 0.954 | 0.897 | 1 |
| MMP8 | | | | | | | | | |
| RSAD2, IFIT1, ISG15, IL1R2, FAM20A, RETN, | 0.857 | 0.848 | 0.867 | 0.905 | 0.842 | 0.969 | 0.945 | 0.872 | 1 |
| MMP8 | | | | | | | | | |
| RSAD2, OAS1, SIGLEC1, ISG15, HERC6, | 0.857 | 0.847 | 0.867 | 0.897 | 0.834 | 0.960 | 0.951 | 0.906 | 0.996 |
| OLAH, MMP8 | | | | | | | | | |
| OAS1, IFI44L, ISG15, IL1R2, FAM20A, RETN, | 0.857 | 0.847 | 0.868 | 0.908 | 0.846 | 0.969 | 0.943 | 0.868 | 1 |
| MMP8 | | | | | | | | | |
| IFIT1, SIGLEC1, ISG15, HERC6, SLC1A2, | 0.857 | 0.848 | 0.867 | 0.902 | 0.842 | 0.962 | 0.938 | 0.884 | 0.992 |
| RETN, MMP8 | | | | | | | | | |
| RSAD2, OAS1, IFIT1, IFI44L, HERC6, FAM20A, | 0.857 | 0.847 | 0.867 | 0.899 | 0.834 | 0.964 | 0.925 | 0.844 | 1 |
| RETN | | | | | | | | | |
| IFIT1, IFI44L, SIGLEC1, ISG15, HERC6, | 0.857 | 0.848 | 0.866 | 0.901 | 0.842 | 0.961 | 0.932 | 0.874 | 0.989 |
| SLC1A2, RETN | | | | | | | | | |
| IFIT1, IFI44L, SIGLEC1, ISG15, OLAH, RETN, | 0.857 | 0.847 | 0.867 | 0.896 | 0.828 | 0.963 | 0.960 | 0.915 | 1 |
| MMP8 | | | | | | | | | |
| RSAD2, ISG15, SLC1A2, IL1R2, FAM20A, | 0.857 | 0.848 | 0.867 | 0.910 | 0.857 | 0.963 | 0.926 | 0.832 | 1 |
| RETN, MMP8 | | | | | | | | | |
| RSAD2, IFI27, OAS1, IFIT1, HERC6, SLC1A2, | 0.857 | 0.846 | 0.868 | 0.905 | 0.846 | 0.964 | 0.934 | 0.851 | 1 |
| FAM20A | | | | | | | | | |
| RSAD2, OAS1, ISG15, HERC6, IL1R2, RETN, | 0.857 | 0.847 | 0.867 | 0.900 | 0.838 | 0.963 | 0.949 | 0.898 | 1 |
| MMP8 | | | | | | | | | |
| RSAD2, IFI27, IFIT1, IFI44L, ISG15, SLC1A2, | 0.857 | 0.847 | 0.867 | 0.901 | 0.837 | 0.965 | 0.947 | 0.882 | 1 |
| RETN | | | | | | | | | |
| RSAD2, IFIT1, ISG15, HERC6, SLC1A2, IL1R2, | 0.857 | 0.848 | 0.866 | 0.904 | 0.846 | 0.962 | 0.943 | 0.890 | 0.996 |
| OLAH | | | | | | | | | |
| OAS1, IFI44L, SIGLEC1, HERC6, SLC1A2, | 0.857 | 0.847 | 0.867 | 0.903 | 0.841 | 0.965 | 0.950 | 0.900 | 1.000 |
| RETN, MMP8 | | | | | | | | | |
| OAS1, IFIT1, HERC6, SLC1A2, IL1R2, OLAH, | 0.857 | 0.847 | 0.867 | 0.903 | 0.845 | 0.960 | 0.951 | 0.904 | 0.998 |
| MMP8 | | | | | | | | | |
| OAS1, ISG15, HERC6, IL1R2, OLAH, RETN, | 0.857 | 0.847 | 0.867 | 0.902 | 0.840 | 0.964 | 0.959 | 0.918 | 1.000 |
| MMP8 | | | | | | | | | |
| RSAD2, IFI27, OAS1, IFIT1, ISG15, FAM20A, | 0.857 | 0.840 | 0.868 | 0.896 | 0.829 | 0.963 | 0.936 | 0.856 | 1 |
| MMP8 | | | | | | | | | |
| OAS1, IFI44L, SIGLEC1, ISG15, HERC6, RETN, | 0.857 | 0.847 | 0.867 | 0.897 | 0.829 | 0.964 | 0.953 | 0.907 | 1.000 |
| MMP8 | | | | | | | | | |
| IFIT1, ISG15, HERC6, SLC1A2, IL1R2, OLAH, | 0.857 | 0.847 | 0.866 | 0.904 | 0.847 | 0.962 | 0.950 | 0.903 | 0.997 |
| MMP8 | | | | | | | | | |
| OAS1, IFIT1, ISG15, HERC6, SLC1A2, IL1R2, | 0.857 | 0.846 | 0.867 | 0.904 | 0.846 | 0.961 | 0.943 | 0.887 | 1.000 |
| RETN | | | | | | | | | |
| RSAD2, IFIT1, ISG15, HERC6, FAM20A, RETN, | 0.857 | 0.847 | 0.867 | 0.904 | 0.841 | 0.966 | 0.936 | 0.859 | 1 |
| MMP8 | | | | | | | | | |
| OAS1, IFIT1, IFI44L, ISG15, HERC6, IL1R2, | 0.857 | 0.847 | 0.867 | 0.898 | 0.835 | 0.961 | 0.952 | 0.904 | 1 |
| RETN | | | | | | | | | |
| RSAD2, OAS1, IFI44L, HERC6, SLC1A2, OLAH, | 0.857 | 0.846 | 0.867 | 0.907 | 0.849 | 0.966 | 0.933 | 0.870 | 0.995 |
| RETN | | | | | | | | | |
| IFI44L, ISG15, HERC6, SLC1A2, IL1R2, OLAH, | 0.857 | 0.847 | 0.867 | 0.909 | 0.852 | 0.965 | 0.942 | 0.888 | 0.997 |
| RETN | | | | | | | | | |
| RSAD2, IFI44L, SIGLEC1, ISG15, HERC6, | 0.857 | 0.847 | 0.867 | 0.897 | 0.830 | 0.964 | 0.950 | 0.903 | 0.997 |
| OLAH, RETN | | | | | | | | | |
| RSAD2, IFIT1, IFI44L, SIGLEC1, HERC6, RETN, | 0.857 | 0.847 | 0.866 | 0.895 | 0.826 | 0.964 | 0.922 | 0.860 | 0.983 |
| MMP8 | | | | | | | | | |
| RSAD2, OAS1, HERC6, IL1R2, OLAH, RETN, | 0.857 | 0.846 | 0.867 | 0.899 | 0.835 | 0.963 | 0.958 | 0.916 | 0.999 |
| MMP8 | | | | | | | | | |
| OAS1, IFIT1, ISG15, HERC6, FAM20A, RETN, | 0.857 | 0.847 | 0.867 | 0.903 | 0.840 | 0.966 | 0.936 | 0.859 | 1 |
| MMP8 | | | | | | | | | |
| RSAD2, OAS1, ISG15, HERC6, SLC1A2, IL1R2, | 0.857 | 0.847 | 0.867 | 0.905 | 0.849 | 0.962 | 0.943 | 0.887 | 1.000 |
| OLAH | | | | | | | | | |
| RSAD2, ISG15, HERC6, IL1R2, OLAH, RETN, | 0.857 | 0.846 | 0.867 | 0.901 | 0.838 | 0.963 | 0.960 | 0.920 | 1.000 |
| MMP8 | | | | | | | | | |
| OAS1, IFIT1, IFI44L, SIGLEC1, ISG15, OLAH, | 0.857 | 0.847 | 0.866 | 0.894 | 0.825 | 0.964 | 0.938 | 0.882 | 0.994 |
| RETN | | | | | | | | | |
| OAS1, ISG15, HERC6, SLC1A2, FAM20A, | 0.857 | 0.846 | 0.867 | 0.903 | 0.842 | 0.965 | 0.936 | 0.857 | 1 |
| RETN, MMP8 | | | | | | | | | |
| IFI44L, ISG15, SLC1A2, IL1R2, OLAH, RETN, | 0.857 | 0.847 | 0.866 | 0.907 | 0.850 | 0.963 | 0.948 | 0.899 | 0.997 |
| MMP8 | | | | | | | | | |
| RSAD2, IFI27, IFIT1, IFI44L, ISG15, HERC6, | 0.857 | 0.847 | 0.866 | 0.898 | 0.840 | 0.956 | 0.893 | 0.813 | 0.974 |
| MMP8 | | | | | | | | | |
| RSAD2, OAS1, IFI44L, SIGLEC1, HERC6, | 0.857 | 0.847 | 0.866 | 0.903 | 0.842 | 0.965 | 0.952 | 0.903 | 1 |
| SLC1A2, RETN | | | | | | | | | |
| OAS1, IFIT1, ISG15, HERC6, SLC1A2, IL1R2, | 0.856 | 0.846 | 0.867 | 0.903 | 0.846 | 0.960 | 0.947 | 0.896 | 0.997 |
| OLAH | | | | | | | | | |
| RSAD2, IFIT1, SIGLEC1, HERC6, SLC1A2, | 0.856 | 0.847 | 0.866 | 0.903 | 0.842 | 0.964 | 0.932 | 0.875 | 0.988 |
| RETN, MMP8 | | | | | | | | | |
| IFIT1, IFI44L, ISG15, HERC6, SLC1A2, OLAH, | 0.856 | 0.846 | 0.866 | 0.908 | 0.851 | 0.966 | 0.934 | 0.874 | 0.994 |
| RETN | | | | | | | | | |
| IFIT1, IFI44L, ISG15, HERC6, SLC1A2, OLAH, | 0.856 | 0.846 | 0.866 | 0.906 | 0.849 | 0.963 | 0.938 | 0.883 | 0.993 |
| MMP8 | | | | | | | | | |
| IFIT1, IFI44L, SIGLEC1, HERC6, SLC1A2, | 0.856 | 0.847 | 0.866 | 0.902 | 0.842 | 0.962 | 0.930 | 0.873 | 0.988 |
| RETN, MMP8 | | | | | | | | | |
| RSAD2, OAS1, IFIT1, HERC6, SLC1A2, IL1R2, | 0.856 | 0.846 | 0.867 | 0.904 | 0.846 | 0.961 | 0.947 | 0.895 | 0.999 |
| OLAH | | | | | | | | | |
| RSAD2, IFIT1, ISG15, HERC6, SLC1A2, | 0.856 | 0.846 | 0.867 | 0.902 | 0.840 | 0.964 | 0.937 | 0.858 | 1 |
| FAM20A, RETN | | | | | | | | | |
| RSAD2, IFIT1, ISG15, SLC1A2, OLAH, | 0.856 | 0.847 | 0.866 | 0.908 | 0.848 | 0.967 | 0.938 | 0.866 | 1 |
| FAM20A, RETN | | | | | | | | | |
| OAS1, IFIT1, ISG15, HERC6, SLC1A2, OLAH, | 0.856 | 0.846 | 0.867 | 0.904 | 0.846 | 0.963 | 0.945 | 0.888 | 1 |
| RETN | | | | | | | | | |
| IFIT1, HERC6, SLC1A2, IL1R2, OLAH, RETN, | 0.856 | 0.846 | 0.866 | 0.906 | 0.849 | 0.964 | 0.955 | 0.911 | 1.000 |
| MMP8 | | | | | | | | | |
| RSAD2, HERC6, SLC1A2, IL1R2, OLAH, RETN, | 0.856 | 0.847 | 0.866 | 0.906 | 0.848 | 0.963 | 0.943 | 0.890 | 0.997 |
| MMP8 | | | | | | | | | |
| RSAD2, ISG15, HERC6, SLC1A2, IL1R2, OLAH, | 0.856 | 0.846 | 0.866 | 0.906 | 0.849 | 0.963 | 0.942 | 0.886 | 0.999 |
| RETN | | | | | | | | | |
| RSAD2, IFIT1, HERC6, SLC1A2, IL1R2, OLAH, | 0.856 | 0.846 | 0.866 | 0.904 | 0.847 | 0.962 | 0.950 | 0.903 | 0.997 |
| MMP8 | | | | | | | | | |
| RSAD2, IFI44L, HERC6, SLC1A2, IL1R2, OLAH, | 0.856 | 0.846 | 0.866 | 0.905 | 0.848 | 0.963 | 0.936 | 0.876 | 0.995 |
| RETN | | | | | | | | | |
| RSAD2, OAS1, IFI44L, ISG15, HERC6, IL1R2, | 0.856 | 0.846 | 0.866 | 0.898 | 0.835 | 0.961 | 0.952 | 0.904 | 1 |
| RETN | | | | | | | | | |
| OAS1, ISG15, HERC6, SLC1A2, IL1R2, OLAH, | 0.856 | 0.846 | 0.867 | 0.906 | 0.849 | 0.963 | 0.936 | 0.871 | 1 |
| RETN | | | | | | | | | |
| OAS1, IFIT1, HERC6, SLC1A2, OLAH, RETN, | 0.856 | 0.846 | 0.866 | 0.901 | 0.840 | 0.962 | 0.951 | 0.904 | 0.998 |
| MMP8 | | | | | | | | | |
| IFIT1, IFI44L, ISG15, SLC1A2, IL1R2, OLAH, | 0.856 | 0.847 | 0.865 | 0.904 | 0.847 | 0.961 | 0.948 | 0.900 | 0.996 |
| MMP8 | | | | | | | | | |
| RSAD2, OAS1, HERC6, SLC1A2, IL1R2, OLAH, | 0.856 | 0.847 | 0.866 | 0.904 | 0.846 | 0.961 | 0.946 | 0.892 | 0.999 |
| MMP8 | | | | | | | | | |
| IFI44L, ISG15, HERC6, SLC1A2, IL1R2, OLAH, | 0.856 | 0.847 | 0.866 | 0.908 | 0.852 | 0.964 | 0.945 | 0.892 | 0.997 |
| MMP8 | | | | | | | | | |
| RSAD2, OAS1, IFI44L, SIGLEC1, HERC6, | 0.856 | 0.846 | 0.866 | 0.897 | 0.834 | 0.960 | 0.948 | 0.900 | 0.995 |
| OLAH, MMP8 | | | | | | | | | |
| OAS1, ISG15, HERC6, SLC1A2, OLAH, RETN, | 0.856 | 0.846 | 0.866 | 0.905 | 0.846 | 0.963 | 0.953 | 0.906 | 1 |
| MMP8 | | | | | | | | | |
| IFIT1, IFI44L, HERC6, SLC1A2, IL1R2, OLAH, | 0.856 | 0.846 | 0.866 | 0.907 | 0.850 | 0.963 | 0.948 | 0.898 | 0.998 |
| MMP8 | | | | | | | | | |
| RSAD2, OAS1, ISG15, HERC6, IL1R2, OLAH, | 0.856 | 0.846 | 0.866 | 0.900 | 0.837 | 0.962 | 0.952 | 0.905 | 0.999 |
| RETN | | | | | | | | | |
| OAS1, IFIT1, IFI44L, SIGLEC1, ISG15, OLAH, | 0.856 | 0.846 | 0.866 | 0.893 | 0.825 | 0.961 | 0.943 | 0.892 | 0.995 |
| MMP8 | | | | | | | | | |
| IFI44L, ISG15, HERC6, SLC1A2, OLAH, RETN, | 0.856 | 0.846 | 0.866 | 0.907 | 0.848 | 0.966 | 0.949 | 0.902 | 0.996 |
| MMP8 | | | | | | | | | |
| OAS1, IFI44L, ISG15, HERC6, IL1R2, RETN, | 0.856 | 0.846 | 0.866 | 0.898 | 0.835 | 0.961 | 0.954 | 0.908 | 1 |
| MMP8 | | | | | | | | | |
| OAS1, IFIT1, IFI44L, HERC6, SLC1A2, OLAH, | 0.856 | 0.846 | 0.866 | 0.905 | 0.848 | 0.962 | 0.935 | 0.877 | 0.992 |
| MMP8 | | | | | | | | | |
| IFI44L, ISG15, HERC6, IL1R2, OLAH, RETN, | 0.856 | 0.846 | 0.866 | 0.900 | 0.836 | 0.963 | 0.963 | 0.925 | 1 |
| MMP8 | | | | | | | | | |
| RSAD2, OAS1, IFIT1, IFI44L, SIGLEC1, OLAH, | 0.856 | 0.846 | 0.866 | 0.895 | 0.825 | 0.964 | 0.945 | 0.891 | 0.998 |
| RETN | | | | | | | | | |
| RSAD2, IFI44L, IL1R2, OLAH, FAM20A, RETN, | 0.856 | 0.846 | 0.866 | 0.906 | 0.844 | 0.967 | 0.953 | 0.893 | 1 |
| MMP8 | | | | | | | | | |
| OAS1, IFIT1, IFI44L, ISG15, HERC6, SLC1A2, | 0.856 | 0.846 | 0.866 | 0.906 | 0.849 | 0.963 | 0.936 | 0.879 | 0.993 |
| OLAH | | | | | | | | | |
| RSAD2, OAS1, HERC6, SLC1A2, IL1R2, OLAH, | 0.856 | 0.846 | 0.866 | 0.903 | 0.845 | 0.962 | 0.939 | 0.876 | 1 |
| RETN | | | | | | | | | |
| RSAD2, IFIT1, IFI44L, HERC6, SLC1A2, OLAH, | 0.856 | 0.846 | 0.866 | 0.908 | 0.851 | 0.965 | 0.928 | 0.867 | 0.990 |
| RETN | | | | | | | | | |
| OAS1, HERC6, SLC1A2, IL1R2, OLAH, RETN, | 0.856 | 0.846 | 0.866 | 0.907 | 0.850 | 0.964 | 0.939 | 0.882 | 0.996 |
| MMP8 | | | | | | | | | |
| RSAD2, IFI27, IFIT1, ISG15, SLC1A2, RETN, | 0.856 | 0.846 | 0.866 | 0.901 | 0.838 | 0.964 | 0.942 | 0.874 | 1 |
| MMP8 | | | | | | | | | |
| OAS1, IFIT1, IFI44L, HERC6, IL1R2, RETN, | 0.856 | 0.846 | 0.866 | 0.895 | 0.827 | 0.962 | 0.959 | 0.918 | 1.000 |
| MMP8 | | | | | | | | | |
| RSAD2, IFI27, IFI44L, ISG15, SLC1A2, RETN, | 0.856 | 0.846 | 0.866 | 0.903 | 0.841 | 0.966 | 0.940 | 0.872 | 1 |
| MMP8 | | | | | | | | | |
| RSAD2, OAS1, ISG15, HERC6, SLC1A2, OLAH, | 0.856 | 0.846 | 0.866 | 0.905 | 0.847 | 0.964 | 0.939 | 0.876 | 1 |
| RETN | | | | | | | | | |
| RSAD2, IFIT1, ISG15, IL1R2, OLAH, FAM20A, | 0.856 | 0.846 | 0.866 | 0.898 | 0.832 | 0.964 | 0.949 | 0.884 | 1 |
| RETN | | | | | | | | | |
| OAS1, IFIT1, HERC6, SLC1A2, IL1R2, OLAH, | 0.856 | 0.845 | 0.866 | 0.904 | 0.846 | 0.962 | 0.940 | 0.883 | 0.997 |
| RETN | | | | | | | | | |
| RSAD2, OAS1, IFIT1, SLC1A2, IL1R2, OLAH, | 0.856 | 0.846 | 0.865 | 0.903 | 0.846 | 0.960 | 0.951 | 0.905 | 0.997 |
| MMP8 | | | | | | | | | |
| RSAD2, IFI27, OAS1, SIGLEC1, ISG15, HERC6, | 0.856 | 0.846 | 0.865 | 0.902 | 0.847 | 0.957 | 0.945 | 0.892 | 0.997 |
| SLC1A2 | | | | | | | | | |
| RSAD2, OAS1, IFIT1, HERC6, IL1R2, RETN, | 0.856 | 0.846 | 0.866 | 0.895 | 0.827 | 0.962 | 0.960 | 0.919 | 1 |
| MMP8 | | | | | | | | | |
| RSAD2, OAS1, IFI44L, HERC6, SLC1A2, IL1R2, | 0.856 | 0.845 | 0.866 | 0.907 | 0.851 | 0.963 | 0.941 | 0.885 | 0.997 |
| OLAH | | | | | | | | | |
| IFI27, OAS1, IFIT1, IFI44L, ISG15, SLC1A2, | 0.856 | 0.845 | 0.866 | 0.900 | 0.834 | 0.965 | 0.941 | 0.863 | 1 |
| FAM20A | | | | | | | | | |
| OAS1, IFI44L, ISG15, HERC6, FAM20A, RETN, | 0.856 | 0.845 | 0.866 | 0.903 | 0.839 | 0.968 | 0.942 | 0.870 | 1 |
| MMP8 | | | | | | | | | |
| RSAD2, OAS1, HERC6, SLC1A2, OLAH, RETN, | 0.856 | 0.846 | 0.865 | 0.902 | 0.842 | 0.963 | 0.947 | 0.894 | 0.999 |
| MMP8 | | | | | | | | | |
| OAS1, IFI44L, SIGLEC1, ISG15, HERC6, OLAH, | 0.856 | 0.846 | 0.866 | 0.896 | 0.833 | 0.959 | 0.953 | 0.909 | 0.998 |
| MMP8 | | | | | | | | | |
| RSAD2, OAS1, IFIT1, SIGLEC1, ISG15, OLAH, | 0.856 | 0.846 | 0.865 | 0.895 | 0.826 | 0.964 | 0.946 | 0.893 | 0.998 |
| RETN | | | | | | | | | |
| OAS1, IFIT1, ISG15, HERC6, IL1R2, OLAH, | 0.856 | 0.845 | 0.866 | 0.900 | 0.837 | 0.962 | 0.950 | 0.902 | 0.998 |
| RETN | | | | | | | | | |
| RSAD2, IFI44L, ISG15, HERC6, SLC1A2, IL1R2, | 0.856 | 0.845 | 0.866 | 0.907 | 0.851 | 0.963 | 0.942 | 0.889 | 0.996 |
| OLAH | | | | | | | | | |
| RSAD2, IFIT1, IFI44L, ISG15, HERC6, IL1R2, | 0.856 | 0.846 | 0.865 | 0.900 | 0.837 | 0.962 | 0.949 | 0.900 | 0.997 |
| RETN | | | | | | | | | |
| IFI27, OAS1, IFI44L, SIGLEC1, ISG15, HERC6, | 0.856 | 0.847 | 0.864 | 0.902 | 0.847 | 0.957 | 0.947 | 0.895 | 0.998 |
| SLC1A2 | | | | | | | | | |
| OAS1, IFI44L, HERC6, SLC1A2, IL1R2, OLAH, | 0.856 | 0.845 | 0.866 | 0.906 | 0.849 | 0.964 | 0.934 | 0.873 | 0.995 |
| RETN | | | | | | | | | |
| RSAD2, IFI44L, ISG15, HERC6, SLC1A2, OLAH, | 0.856 | 0.845 | 0.866 | 0.909 | 0.852 | 0.966 | 0.939 | 0.885 | 0.994 |
| RETN | | | | | | | | | |
| OAS1, IFIT1, HERC6, IL1R2, OLAH, RETN, | 0.856 | 0.845 | 0.866 | 0.899 | 0.835 | 0.963 | 0.962 | 0.924 | 1 |
| MMP8 | | | | | | | | | |
| RSAD2, IFIT1, ISG15, HERC6, IL1R2, RETN, | 0.856 | 0.845 | 0.866 | 0.897 | 0.832 | 0.962 | 0.957 | 0.913 | 1.000 |
| MMP8 | | | | | | | | | |
| IFIT1, IFI44L, ISG15, HERC6, SLC1A2, IL1R2, | 0.855 | 0.846 | 0.865 | 0.907 | 0.851 | 0.963 | 0.945 | 0.892 | 0.997 |
| OLAH | | | | | | | | | |
| RSAD2, ISG15, HERC6, SLC1A2, IL1R2, OLAH, | 0.855 | 0.846 | 0.865 | 0.906 | 0.849 | 0.963 | 0.946 | 0.893 | 0.998 |
| MMP8 | | | | | | | | | |
| RSAD2, OAS1, IFI44L, ISG15, HERC6, SLC1A2, | 0.855 | 0.845 | 0.866 | 0.900 | 0.839 | 0.960 | 0.924 | 0.841 | 1 |
| FAM20A | | | | | | | | | |
| RSAD2, OAS1, IFIT1, IFI44L, SIGLEC1, OLAH, | 0.855 | 0.846 | 0.865 | 0.893 | 0.825 | 0.961 | 0.946 | 0.895 | 0.996 |
| MMP8 | | | | | | | | | |
| OAS1, IFIT1, IFI44L, ISG15, HERC6, OLAH, | 0.855 | 0.845 | 0.865 | 0.897 | 0.832 | 0.961 | 0.946 | 0.896 | 0.995 |
| RETN | | | | | | | | | |
| RSAD2, ISG15, HERC6, SLC1A2, FAM20A, | 0.855 | 0.845 | 0.866 | 0.903 | 0.841 | 0.965 | 0.935 | 0.856 | 1 |
| RETN, MMP8 | | | | | | | | | |
| IFIT1, IFI44L, ISG15, HERC6, IL1R2, RETN, | 0.855 | 0.846 | 0.865 | 0.895 | 0.830 | 0.961 | 0.960 | 0.919 | 1 |
| MMP8 | | | | | | | | | |
| IFIT1, ISG15, HERC6, SLC1A2, IL1R2, OLAH, | 0.855 | 0.845 | 0.865 | 0.906 | 0.849 | 0.964 | 0.943 | 0.888 | 0.999 |
| RETN | | | | | | | | | |
| OAS1, ISG15, HERC6, SLC1A2, IL1R2, OLAH, | 0.855 | 0.846 | 0.865 | 0.905 | 0.849 | 0.962 | 0.942 | 0.888 | 0.996 |
| MMP8 | | | | | | | | | |
| RSAD2, IFIT1, IFI44L, SIGLEC1, OLAH, RETN, | 0.855 | 0.845 | 0.865 | 0.896 | 0.828 | 0.963 | 0.959 | 0.916 | 1 |
| MMP8 | | | | | | | | | |
| RSAD2, IFI44L, HERC6, SLC1A2, IL1R2, OLAH, | 0.855 | 0.845 | 0.865 | 0.908 | 0.852 | 0.963 | 0.945 | 0.893 | 0.996 |
| MMP8 | | | | | | | | | |
| RSAD2, OAS1, IFI44L, ISG15, OLAH, FAM20A, | 0.855 | 0.844 | 0.866 | 0.898 | 0.830 | 0.966 | 0.948 | 0.889 | 1 |
| MMP8 | | | | | | | | | |
| OAS1, IFI44L, ISG15, HERC6, SLC1A2, IL1R2, | 0.855 | 0.845 | 0.866 | 0.907 | 0.852 | 0.962 | 0.939 | 0.881 | 0.997 |
| OLAH | | | | | | | | | |
| RSAD2, OAS1, ISG15, HERC6, OLAH, RETN, | 0.855 | 0.845 | 0.866 | 0.896 | 0.830 | 0.962 | 0.959 | 0.918 | 0.999 |
| MMP8 | | | | | | | | | |
| RSAD2, IFI44L, ISG15, IL1R2, OLAH, FAM20A, | 0.855 | 0.845 | 0.866 | 0.900 | 0.835 | 0.965 | 0.948 | 0.885 | 1 |
| RETN | | | | | | | | | |
| IFIT1, IFI44L, ISG15, SLC1A2, IL1R2, OLAH, | 0.855 | 0.845 | 0.865 | 0.910 | 0.854 | 0.966 | 0.946 | 0.896 | 0.995 |
| RETN | | | | | | | | | |
| RSAD2, OAS1, IFI44L, HERC6, SLC1A2, OLAH, | 0.855 | 0.845 | 0.866 | 0.905 | 0.849 | 0.962 | 0.938 | 0.883 | 0.993 |
| MMP8 | | | | | | | | | |
| IFIT1, ISG15, HERC6, IL1R2, OLAH, RETN, | 0.855 | 0.845 | 0.865 | 0.900 | 0.837 | 0.963 | 0.962 | 0.924 | 1 |
| MMP8 | | | | | | | | | |
| IFIT1, IFI44L, HERC6, SLC1A2, IL1R2, OLAH, | 0.855 | 0.845 | 0.865 | 0.907 | 0.850 | 0.964 | 0.936 | 0.876 | 0.995 |
| RETN | | | | | | | | | |
| OAS1, IFI44L, ISG15, HERC6, SLC1A2, IL1R2, | 0.855 | 0.845 | 0.865 | 0.906 | 0.850 | 0.962 | 0.942 | 0.883 | 1 |
| RETN | | | | | | | | | |
| OAS1, IFIT1, HERC6, SLC1A2, IL1R2, RETN, | 0.855 | 0.845 | 0.865 | 0.904 | 0.846 | 0.963 | 0.952 | 0.903 | 1 |
| MMP8 | | | | | | | | | |
| OAS1, IFI44L, ISG15, HERC6, IL1R2, OLAH, | 0.855 | 0.845 | 0.865 | 0.899 | 0.836 | 0.962 | 0.952 | 0.905 | 0.999 |
| RETN | | | | | | | | | |
| IFI27, OAS1, IFIT1, IFI44L, SIGLEC1, ISG15, | 0.855 | 0.845 | 0.865 | 0.890 | 0.824 | 0.956 | 0.912 | 0.837 | 0.987 |
| MMP8 | | | | | | | | | |
| RSAD2, IFIT1, IFI44L, IL1R2, FAM20A, RETN, | 0.855 | 0.845 | 0.865 | 0.904 | 0.841 | 0.967 | 0.946 | 0.873 | 1 |
| MMP8 | | | | | | | | | |
| OAS1, IFI44L, HERC6, SLC1A2, IL1R2, OLAH, | 0.855 | 0.845 | 0.865 | 0.908 | 0.853 | 0.963 | 0.942 | 0.888 | 0.997 |
| MMP8 | | | | | | | | | |
| OAS1, IFIT1, ISG15, SLC1A2, IL1R2, OLAH, | 0.855 | 0.846 | 0.864 | 0.904 | 0.846 | 0.961 | 0.948 | 0.899 | 0.996 |
| MMP8 | | | | | | | | | |
| RSAD2, IFI44L, SIGLEC1, ISG15, HERC6, | 0.855 | 0.846 | 0.864 | 0.907 | 0.847 | 0.967 | 0.945 | 0.893 | 0.996 |
| SLC1A2, RETN | | | | | | | | | |
| RSAD2, IFI44L, HERC6, SLC1A2, OLAH, RETN, | 0.855 | 0.845 | 0.865 | 0.905 | 0.846 | 0.965 | 0.937 | 0.882 | 0.992 |
| MMP8 | | | | | | | | | |
| OAS1, IFIT1, ISG15, HERC6, SLC1A2, IL1R2, | 0.855 | 0.845 | 0.865 | 0.903 | 0.846 | 0.959 | 0.952 | 0.903 | 1 |
| MMP8 | | | | | | | | | |
| RSAD2, OAS1, IFIT1, HERC6, SLC1A2, IL1R2, | 0.855 | 0.845 | 0.864 | 0.902 | 0.845 | 0.959 | 0.951 | 0.902 | 1 |
| MMP8 | | | | | | | | | |
| RSAD2, OAS1, IFIT1, ISG15, HERC6, SLC1A2, | 0.855 | 0.845 | 0.864 | 0.901 | 0.844 | 0.958 | 0.951 | 0.901 | 1 |
| IL1R2 | | | | | | | | | |
| ISG15, HERC6, SLC1A2, IL1R2, OLAH, RETN, | 0.855 | 0.845 | 0.864 | 0.908 | 0.853 | 0.964 | 0.951 | 0.902 | 1 |
| MMP8 | | | | | | | | | |
| OAS1, IFIT1, SLC1A2, IL1R2, OLAH, RETN, | 0.855 | 0.845 | 0.865 | 0.905 | 0.847 | 0.963 | 0.957 | 0.914 | 0.999 |
| MMP8 | | | | | | | | | |
| RSAD2, OAS1, ISG15, OLAH, FAM20A, RETN, | 0.855 | 0.844 | 0.865 | 0.897 | 0.829 | 0.966 | 0.952 | 0.894 | 1 |
| MMP8 | | | | | | | | | |
| RSAD2, IFIT1, IFI44L, HERC6, SLC1A2, OLAH, | 0.855 | 0.845 | 0.865 | 0.905 | 0.848 | 0.962 | 0.932 | 0.873 | 0.990 |
| MMP8 | | | | | | | | | |
| IFIT1, IFI44L, SLC1A2, IL1R2, OLAH, RETN, | 0.855 | 0.845 | 0.864 | 0.906 | 0.848 | 0.964 | 0.954 | 0.911 | 0.998 |
| MMP8 | | | | | | | | | |
| IFIT1, IFI44L, ISG15, SLC1A2, OLAH, RETN, | 0.855 | 0.845 | 0.864 | 0.906 | 0.846 | 0.967 | 0.948 | 0.900 | 0.995 |
| MMP8 | | | | | | | | | |
| RSAD2, IFI27, OAS1, IFIT1, SIGLEC1, HERC6, | 0.855 | 0.845 | 0.864 | 0.902 | 0.847 | 0.957 | 0.945 | 0.892 | 0.997 |
| SLC1A2 | | | | | | | | | |
| RSAD2, OAS1, IFIT1, IFI44L, HERC6, SLC1A2, | 0.855 | 0.845 | 0.865 | 0.905 | 0.848 | 0.962 | 0.933 | 0.873 | 0.992 |
| OLAH | | | | | | | | | |
| OAS1, IFI44L, ISG15, HERC6, SLC1A2, OLAH, | 0.855 | 0.844 | 0.865 | 0.905 | 0.848 | 0.963 | 0.936 | 0.879 | 0.993 |
| MMP8 | | | | | | | | | |
| RSAD2, IFIT1, IFI44L, HERC6, SLC1A2, IL1R2, | 0.855 | 0.845 | 0.865 | 0.905 | 0.849 | 0.962 | 0.943 | 0.891 | 0.996 |
| OLAH | | | | | | | | | |
| RSAD2, OAS1, IFIT1, IFI44L, ISG15, OLAH, | 0.855 | 0.844 | 0.865 | 0.895 | 0.825 | 0.965 | 0.945 | 0.886 | 1 |
| FAM20A | | | | | | | | | |
| RSAD2, OAS1, IFI44L, ISG15, HERC6, SLC1A2, | 0.855 | 0.844 | 0.865 | 0.907 | 0.850 | 0.963 | 0.937 | 0.881 | 0.993 |
| OLAH | | | | | | | | | |
| OAS1, IFIT1, ISG15, SLC1A2, IL1R2, OLAH, | 0.855 | 0.844 | 0.865 | 0.908 | 0.850 | 0.965 | 0.946 | 0.895 | 0.996 |
| RETN | | | | | | | | | |
| OAS1, IFIT1, IFI44L, HERC6, SLC1A2, | 0.855 | 0.845 | 0.865 | 0.902 | 0.840 | 0.964 | 0.926 | 0.843 | 1 |
| FAM20A, RETN | | | | | | | | | |
| RSAD2, IFI27, OAS1, IFIT1, IFI44L, SIGLEC1, | 0.855 | 0.845 | 0.864 | 0.891 | 0.824 | 0.957 | 0.917 | 0.843 | 0.992 |
| MMP8 | | | | | | | | | |
| IFIT1, IFI44L, HERC6, IL1R2, OLAH, RETN, | 0.855 | 0.844 | 0.865 | 0.899 | 0.835 | 0.963 | 0.962 | 0.924 | 1 |
| MMP8 | | | | | | | | | |
| RSAD2, OAS1, IFIT1, ISG15, OLAH, FAM20A, | 0.855 | 0.844 | 0.865 | 0.892 | 0.822 | 0.962 | 0.951 | 0.890 | 1 |
| MMP8 | | | | | | | | | |
| RSAD2, OAS1, IFIT1, OLAH, FAM20A, RETN, | 0.854 | 0.844 | 0.865 | 0.893 | 0.823 | 0.963 | 0.953 | 0.896 | 1 |
| MMP8 | | | | | | | | | |
| RSAD2, OAS1, ISG15, HERC6, SLC1A2, IL1R2, | 0.854 | 0.844 | 0.864 | 0.901 | 0.843 | 0.958 | 0.938 | 0.873 | 1 |
| RETN | | | | | | | | | |
| RSAD2, OAS1, ISG15, HERC6, IL1R2, OLAH, | 0.854 | 0.845 | 0.864 | 0.899 | 0.840 | 0.958 | 0.948 | 0.900 | 0.996 |
| MMP8 | | | | | | | | | |
| IFIT1, ISG15, HERC6, SLC1A2, OLAH, RETN, | 0.854 | 0.844 | 0.865 | 0.907 | 0.848 | 0.965 | 0.954 | 0.909 | 1.000 |
| MMP8 | | | | | | | | | |
| OAS1, IFIT1, ISG15, HERC6, OLAH, RETN, | 0.854 | 0.844 | 0.865 | 0.895 | 0.828 | 0.962 | 0.960 | 0.920 | 1.000 |
| MMP8 | | | | | | | | | |
| RSAD2, IFI27, IFIT1, IFI44L, SLC1A2, RETN, | 0.854 | 0.844 | 0.864 | 0.902 | 0.838 | 0.965 | 0.941 | 0.873 | 1 |
| MMP8 | | | | | | | | | |
| RSAD2, IFIT1, IFI44L, ISG15, HERC6, SLC1A2, | 0.854 | 0.845 | 0.864 | 0.907 | 0.851 | 0.964 | 0.936 | 0.880 | 0.992 |
| OLAH | | | | | | | | | |
| RSAD2, IFIT1, ISG15, SLC1A2, IL1R2, OLAH, | 0.854 | 0.845 | 0.863 | 0.904 | 0.846 | 0.961 | 0.950 | 0.903 | 0.997 |
| MMP8 | | | | | | | | | |
| RSAD2, OAS1, IFIT1, IFI44L, SLC1A2, OLAH, | 0.854 | 0.844 | 0.864 | 0.906 | 0.848 | 0.964 | 0.938 | 0.884 | 0.992 |
| MMP8 | | | | | | | | | |
| IFI27, IFIT1, IFI44L, ISG15, HERC6, SLC1A2, | 0.854 | 0.845 | 0.864 | 0.900 | 0.842 | 0.957 | 0.899 | 0.816 | 0.982 |
| MMP8 | | | | | | | | | |
| RSAD2, IFIT1, ISG15, HERC6, SLC1A2, IL1R2, | 0.854 | 0.845 | 0.864 | 0.904 | 0.846 | 0.962 | 0.946 | 0.891 | 1.000 |
| RETN | | | | | | | | | |
| RSAD2, OAS1, IFIT1, IFI44L, SIGLEC1, ISG15, | 0.854 | 0.845 | 0.864 | 0.894 | 0.826 | 0.962 | 0.945 | 0.893 | 0.996 |
| OLAH | | | | | | | | | |
| RSAD2, OAS1, IFIT1, ISG15, OLAH, FAM20A, | 0.854 | 0.844 | 0.865 | 0.895 | 0.824 | 0.965 | 0.950 | 0.889 | 1 |
| RETN | | | | | | | | | |
| RSAD2, IFI27, OAS1, ISG15, SLC1A2, FAM20A, | 0.854 | 0.844 | 0.865 | 0.895 | 0.830 | 0.960 | 0.929 | 0.846 | 1 |
| MMP8 | | | | | | | | | |
| RSAD2, OAS1, IFIT1, HERC6, SLC1A2, OLAH, | 0.854 | 0.844 | 0.864 | 0.901 | 0.841 | 0.961 | 0.940 | 0.879 | 1 |
| RETN | | | | | | | | | |
| RSAD2, OAS1, IFIT1, ISG15, HERC6, IL1R2, | 0.854 | 0.845 | 0.864 | 0.897 | 0.838 | 0.957 | 0.949 | 0.900 | 0.997 |
| MMP8 | | | | | | | | | |
| RSAD2, IFIT1, HERC6, SLC1A2, IL1R2, OLAH, | 0.854 | 0.844 | 0.864 | 0.904 | 0.846 | 0.962 | 0.940 | 0.883 | 0.997 |
| RETN | | | | | | | | | |
| IFI27, OAS1, IFIT1, ISG15, SLC1A2, FAM20A, | 0.854 | 0.843 | 0.865 | 0.897 | 0.832 | 0.962 | 0.933 | 0.852 | 1 |
| MMP8 | | | | | | | | | |
| RSAD2, ISG15, HERC6, SLC1A2, OLAH, RETN, | 0.854 | 0.844 | 0.864 | 0.904 | 0.846 | 0.963 | 0.950 | 0.902 | 0.998 |
| MMP8 | | | | | | | | | |
| RSAD2, IFIT1, SIGLEC1, ISG15, OLAH, RETN, | 0.854 | 0.844 | 0.864 | 0.896 | 0.828 | 0.964 | 0.963 | 0.922 | 1 |
| MMP8 | | | | | | | | | |
| RSAD2, OAS1, IFIT1, IFI44L, HERC6, IL1R2, | 0.854 | 0.845 | 0.864 | 0.895 | 0.833 | 0.958 | 0.949 | 0.902 | 0.996 |
| MMP8 | | | | | | | | | |
| IFI27, OAS1, IFIT1, IFI44L, SIGLEC1, HERC6, | 0.854 | 0.845 | 0.863 | 0.901 | 0.846 | 0.957 | 0.946 | 0.893 | 0.998 |
| SLC1A2 | | | | | | | | | |
| RSAD2, IFIT1, IFI44L, HERC6, SLC1A2, | 0.854 | 0.844 | 0.864 | 0.903 | 0.842 | 0.963 | 0.923 | 0.840 | 1 |
| FAM20A, RETN | | | | | | | | | |
| OAS1, IFIT1, IFI44L, HERC6, FAM20A, RETN, | 0.854 | 0.844 | 0.864 | 0.897 | 0.829 | 0.966 | 0.933 | 0.853 | 1 |
| MMP8 | | | | | | | | | |
| RSAD2, OAS1, IFIT1, HERC6, IL1R2, OLAH, | 0.854 | 0.844 | 0.864 | 0.899 | 0.839 | 0.959 | 0.954 | 0.911 | 0.998 |
| MMP8 | | | | | | | | | |
| RSAD2, OAS1, IFIT1, IFI44L, HERC6, IL1R2, | 0.854 | 0.844 | 0.864 | 0.893 | 0.827 | 0.960 | 0.946 | 0.896 | 0.995 |
| RETN | | | | | | | | | |
| IFIT1, IFI44L, ISG15, HERC6, OLAH, RETN, | 0.854 | 0.844 | 0.864 | 0.895 | 0.826 | 0.964 | 0.955 | 0.913 | 0.998 |
| MMP8 | | | | | | | | | |
| RSAD2, IFIT1, HERC6, SLC1A2, OLAH, RETN, | 0.854 | 0.844 | 0.864 | 0.902 | 0.841 | 0.963 | 0.950 | 0.903 | 0.997 |
| MMP8 | | | | | | | | | |
| RSAD2, IFIT1, IFI44L, ISG15, HERC6, SLC1A2, | 0.854 | 0.844 | 0.864 | 0.903 | 0.844 | 0.961 | 0.914 | 0.825 | 1 |
| FAM20A | | | | | | | | | |
| RSAD2, IFI44L, ISG15, HERC6, IL1R2, RETN, | 0.854 | 0.844 | 0.864 | 0.899 | 0.836 | 0.962 | 0.955 | 0.911 | 1 |
| MMP8 | | | | | | | | | |
| IFIT1, IFI44L, ISG15, HERC6, IL1R2, OLAH, | 0.854 | 0.844 | 0.864 | 0.900 | 0.836 | 0.964 | 0.948 | 0.899 | 0.996 |
| RETN | | | | | | | | | |
| RSAD2, IFI44L, ISG15, HERC6, IL1R2, OLAH, | 0.854 | 0.844 | 0.864 | 0.901 | 0.839 | 0.964 | 0.950 | 0.903 | 0.997 |
| RETN | | | | | | | | | |
| RSAD2, OAS1, IFIT1, HERC6, SLC1A2, OLAH, | 0.854 | 0.844 | 0.864 | 0.903 | 0.845 | 0.960 | 0.949 | 0.898 | 1.000 |
| MMP8 | | | | | | | | | |
| OAS1, IFIT1, IFI44L, HERC6, OLAH, RETN, | 0.854 | 0.844 | 0.864 | 0.893 | 0.824 | 0.962 | 0.955 | 0.913 | 0.998 |
| MMP8 | | | | | | | | | |
| RSAD2, IFI44L, ISG15, HERC6, SLC1A2, | 0.854 | 0.843 | 0.865 | 0.902 | 0.843 | 0.962 | 0.923 | 0.840 | 1 |
| FAM20A, MMP8 | | | | | | | | | |
| RSAD2, IFIT1, ISG15, HERC6, SLC1A2, IL1R2, | 0.854 | 0.844 | 0.863 | 0.902 | 0.845 | 0.959 | 0.952 | 0.904 | 1 |
| MMP8 | | | | | | | | | |
| RSAD2, OAS1, IFIT1, HERC6, SLC1A2, IL1R2, | 0.854 | 0.844 | 0.864 | 0.901 | 0.843 | 0.960 | 0.939 | 0.878 | 1 |
| RETN | | | | | | | | | |
| RSAD2, IFI27, IFIT1, ISG15, SLC1A2, FAM20A, | 0.854 | 0.843 | 0.864 | 0.896 | 0.831 | 0.961 | 0.934 | 0.853 | 1 |
| MMP8 | | | | | | | | | |
| OAS1, IFI44L, HERC6, IL1R2, OLAH, RETN, | 0.854 | 0.844 | 0.864 | 0.899 | 0.836 | 0.963 | 0.960 | 0.920 | 0.999 |
| MMP8 | | | | | | | | | |
| OAS1, ISG15, HERC6, SLC1A2, IL1R2, RETN, | 0.854 | 0.844 | 0.864 | 0.904 | 0.847 | 0.960 | 0.934 | 0.864 | 1 |
| MMP8 | | | | | | | | | |
| RSAD2, OAS1, IFI44L, HERC6, IL1R2, OLAH, | 0.854 | 0.844 | 0.864 | 0.898 | 0.833 | 0.963 | 0.948 | 0.899 | 0.997 |
| RETN | | | | | | | | | |
| RSAD2, OAS1, IFI44L, SIGLEC1, ISG15, | 0.854 | 0.844 | 0.864 | 0.898 | 0.835 | 0.961 | 0.952 | 0.907 | 0.997 |
| HERC6, OLAH | | | | | | | | | |
| RSAD2, OAS1, IFIT1, SIGLEC1, ISG15, OLAH, | 0.854 | 0.844 | 0.864 | 0.894 | 0.826 | 0.962 | 0.943 | 0.892 | 0.995 |
| MMP8 | | | | | | | | | |
| RSAD2, OAS1, IFI44L, HERC6, SLC1A2, | 0.854 | 0.843 | 0.864 | 0.901 | 0.839 | 0.963 | 0.929 | 0.850 | 1 |
| FAM20A, RETN | | | | | | | | | |
| RSAD2, IFIT1, IFI44L, HERC6, IL1R2, RETN, | 0.854 | 0.844 | 0.864 | 0.894 | 0.826 | 0.962 | 0.960 | 0.920 | 1.000 |
| MMP8 | | | | | | | | | |
| OAS1, IFIT1, IFI44L, HERC6, SLC1A2, IL1R2, | 0.854 | 0.843 | 0.864 | 0.907 | 0.851 | 0.963 | 0.945 | 0.892 | 0.997 |
| OLAH | | | | | | | | | |
| RSAD2, IFI44L, ISG15, HERC6, SLC1A2, IL1R2, | 0.854 | 0.844 | 0.863 | 0.907 | 0.852 | 0.963 | 0.943 | 0.886 | 1 |
| RETN | | | | | | | | | |
| RSAD2, IFI27, OAS1, IFI44L, SIGLEC1, HERC6, | 0.854 | 0.845 | 0.863 | 0.901 | 0.846 | 0.957 | 0.947 | 0.895 | 0.999 |
| SLC1A2 | | | | | | | | | |
| RSAD2, OAS1, IFI44L, OLAH, FAM20A, RETN, | 0.854 | 0.843 | 0.864 | 0.901 | 0.835 | 0.967 | 0.946 | 0.887 | 1 |
| MMP8 | | | | | | | | | |
| RSAD2, IFIT1, IFI44L, SLC1A2, IL1R2, OLAH, | 0.854 | 0.844 | 0.863 | 0.905 | 0.849 | 0.961 | 0.950 | 0.903 | 0.997 |
| MMP8 | | | | | | | | | |
| IFIT1, ISG15, SLC1A2, IL1R2, OLAH, RETN, | 0.854 | 0.844 | 0.863 | 0.907 | 0.849 | 0.964 | 0.957 | 0.914 | 0.999 |
| MMP8 | | | | | | | | | |
| OAS1, IFIT1, IFI44L, HERC6, SLC1A2, IL1R2, | 0.854 | 0.844 | 0.863 | 0.903 | 0.847 | 0.959 | 0.951 | 0.903 | 1.000 |
| MMP8 | | | | | | | | | |
| IFIT1, IFI44L, HERC6, SLC1A2, FAM20A, | 0.854 | 0.843 | 0.864 | 0.901 | 0.838 | 0.964 | 0.930 | 0.849 | 1 |
| RETN, MMP8 | | | | | | | | | |
| RSAD2, IFIT1, IFI44L, ISG15, IL1R2, FAM20A, | 0.854 | 0.844 | 0.864 | 0.897 | 0.832 | 0.962 | 0.945 | 0.875 | 1 |
| RETN | | | | | | | | | |
| RSAD2, IFIT1, IFI44L, ISG15, HERC6, SLC1A2, | 0.854 | 0.844 | 0.863 | 0.901 | 0.845 | 0.958 | 0.950 | 0.900 | 1.000 |
| IL1R2 | | | | | | | | | |
| RSAD2, IFIT1, ISG15, HERC6, IL1R2, OLAH, | 0.854 | 0.844 | 0.864 | 0.899 | 0.836 | 0.962 | 0.951 | 0.905 | 0.997 |
| RETN | | | | | | | | | |
| RSAD2, OAS1, IFI44L, SIGLEC1, OLAH, RETN, | 0.854 | 0.843 | 0.864 | 0.893 | 0.824 | 0.963 | 0.958 | 0.916 | 0.999 |
| MMP8 | | | | | | | | | |
| OAS1, IFIT1, IFI44L, ISG15, SLC1A2, OLAH, | 0.854 | 0.844 | 0.863 | 0.904 | 0.845 | 0.962 | 0.938 | 0.884 | 0.992 |
| MMP8 | | | | | | | | | |
| RSAD2, OAS1, IFIT1, ISG15, HERC6, IL1R2, | 0.854 | 0.844 | 0.863 | 0.899 | 0.839 | 0.959 | 0.951 | 0.905 | 0.997 |
| OLAH | | | | | | | | | |
| RSAD2, IFI27, IFI44L, ISG15, HERC6, SLC1A2, | 0.854 | 0.844 | 0.863 | 0.900 | 0.843 | 0.957 | 0.904 | 0.827 | 0.982 |
| MMP8 | | | | | | | | | |
| IFIT1, ISG15, HERC6, SLC1A2, FAM20A, | 0.854 | 0.843 | 0.864 | 0.901 | 0.838 | 0.964 | 0.940 | 0.864 | 1 |
| RETN, MMP8 | | | | | | | | | |
| RSAD2, IFIT1, ISG15, HERC6, SLC1A2, OLAH, | 0.853 | 0.843 | 0.864 | 0.904 | 0.845 | 0.963 | 0.943 | 0.888 | 0.999 |
| RETN | | | | | | | | | |
| RSAD2, IFI27, OAS1, IFIT1, SLC1A2, FAM20A, | 0.853 | 0.843 | 0.864 | 0.897 | 0.832 | 0.962 | 0.933 | 0.852 | 1 |
| MMP8 | | | | | | | | | |
| RSAD2, IFI27, IFIT1, IFI44L, HERC6, SLC1A2, | 0.853 | 0.844 | 0.863 | 0.901 | 0.844 | 0.958 | 0.899 | 0.818 | 0.980 |
| MMP8 | | | | | | | | | |
| OAS1, IFIT1, IFI44L, SLC1A2, IL1R2, OLAH, | 0.853 | 0.844 | 0.863 | 0.904 | 0.847 | 0.961 | 0.950 | 0.903 | 0.997 |
| MMP8 | | | | | | | | | |
| RSAD2, IFI44L, ISG15, HERC6, SLC1A2, OLAH, | 0.853 | 0.843 | 0.863 | 0.906 | 0.849 | 0.963 | 0.936 | 0.880 | 0.991 |
| MMP8 | | | | | | | | | |
| RSAD2, OAS1, IFI44L, ISG15, HERC6, OLAH, | 0.853 | 0.843 | 0.864 | 0.897 | 0.833 | 0.962 | 0.949 | 0.902 | 0.996 |
| RETN | | | | | | | | | |
| RSAD2, IFI44L, SIGLEC1, ISG15, HERC6, | 0.853 | 0.844 | 0.863 | 0.899 | 0.836 | 0.961 | 0.952 | 0.907 | 0.997 |
| OLAH, MMP8 | | | | | | | | | |
| OAS1, IFIT1, ISG15, HERC6, SLC1A2, OLAH, | 0.853 | 0.843 | 0.864 | 0.903 | 0.845 | 0.960 | 0.945 | 0.893 | 0.996 |
| MMP8 | | | | | | | | | |
| RSAD2, OAS1, SIGLEC1, ISG15, OLAH, RETN, | 0.853 | 0.843 | 0.864 | 0.892 | 0.822 | 0.962 | 0.960 | 0.919 | 1 |
| MMP8 | | | | | | | | | |
| RSAD2, OAS1, IFI44L, ISG15, OLAH, FAM20A, | 0.853 | 0.843 | 0.864 | 0.892 | 0.821 | 0.964 | 0.946 | 0.887 | 1 |
| RETN | | | | | | | | | |
| OAS1, IFIT1, IFI44L, ISG15, SLC1A2, OLAH, | 0.853 | 0.843 | 0.863 | 0.909 | 0.851 | 0.967 | 0.936 | 0.881 | 0.991 |
| RETN | | | | | | | | | |
| RSAD2, IFI27, OAS1, IFI44L, SIGLEC1, ISG15, | 0.853 | 0.843 | 0.863 | 0.890 | 0.824 | 0.957 | 0.920 | 0.848 | 0.991 |
| MMP8 | | | | | | | | | |
| RSAD2, OAS1, IFIT1, ISG15, HERC6, OLAH, | 0.853 | 0.843 | 0.863 | 0.897 | 0.832 | 0.961 | 0.952 | 0.906 | 0.998 |
| RETN | | | | | | | | | |
| RSAD2, OAS1, IFIT1, SLC1A2, IL1R2, OLAH, | 0.853 | 0.843 | 0.863 | 0.907 | 0.850 | 0.965 | 0.947 | 0.896 | 0.997 |
| RETN | | | | | | | | | |
| RSAD2, OAS1, IFI44L, HERC6, FAM20A, | 0.853 | 0.843 | 0.864 | 0.896 | 0.828 | 0.963 | 0.930 | 0.852 | 1 |
| RETN, MMP8 | | | | | | | | | |
| RSAD2, OAS1, IFIT1, ISG15, SLC1A2, IL1R2, | 0.853 | 0.843 | 0.863 | 0.905 | 0.847 | 0.963 | 0.947 | 0.897 | 0.997 |
| OLAH | | | | | | | | | |
| RSAD2, IFIT1, HERC6, IL1R2, OLAH, RETN, | 0.853 | 0.843 | 0.863 | 0.898 | 0.834 | 0.963 | 0.961 | 0.922 | 1.000 |
| MMP8 | | | | | | | | | |
| OAS1, IFIT1, IFI44L, HERC6, SLC1A2, IL1R2, | 0.853 | 0.843 | 0.863 | 0.902 | 0.843 | 0.961 | 0.942 | 0.886 | 0.999 |
| RETN | | | | | | | | | |
| IFI44L, ISG15, HERC6, SLC1A2, FAM20A, | 0.853 | 0.843 | 0.864 | 0.898 | 0.832 | 0.964 | 0.945 | 0.867 | 1 |
| RETN, MMP8 | | | | | | | | | |
| RSAD2, OAS1, IFIT1, HERC6, IL1R2, OLAH, | 0.853 | 0.843 | 0.863 | 0.898 | 0.834 | 0.962 | 0.947 | 0.897 | 0.996 |
| RETN | | | | | | | | | |
| OAS1, IFIT1, IFI44L, ISG15, SLC1A2, IL1R2, | 0.853 | 0.843 | 0.863 | 0.910 | 0.854 | 0.966 | 0.945 | 0.893 | 0.996 |
| OLAH | | | | | | | | | |
| IFIT1, IFI44L, HERC6, SLC1A2, IL1R2, RETN, | 0.853 | 0.844 | 0.863 | 0.903 | 0.845 | 0.962 | 0.950 | 0.900 | 1.000 |
| MMP8 | | | | | | | | | |
| OAS1, IFIT1, IFI44L, ISG15, HERC6, SLC1A2, | 0.853 | 0.843 | 0.863 | 0.903 | 0.847 | 0.959 | 0.950 | 0.900 | 1 |
| IL1R2 | | | | | | | | | |
| OAS1, IFI44L, ISG15, HERC6, OLAH, RETN, | 0.853 | 0.843 | 0.864 | 0.895 | 0.828 | 0.962 | 0.964 | 0.927 | 1 |
| MMP8 | | | | | | | | | |
| OAS1, IFIT1, IFI44L, HERC6, IL1R2, OLAH, | 0.853 | 0.843 | 0.863 | 0.899 | 0.838 | 0.959 | 0.954 | 0.911 | 0.998 |
| MMP8 | | | | | | | | | |
| RSAD2, IFI44L, ISG15, IL1R2, FAM20A, RETN, | 0.853 | 0.843 | 0.864 | 0.901 | 0.839 | 0.964 | 0.939 | 0.861 | 1 |
| MMP8 | | | | | | | | | |
| RSAD2, IFI44L, HERC6, SLC1A2, FAM20A, | 0.853 | 0.843 | 0.864 | 0.899 | 0.835 | 0.963 | 0.933 | 0.854 | 1 |
| RETN, MMP8 | | | | | | | | | |
| IFIT1, IFI44L, ISG15, HERC6, SLC1A2, IL1R2, | 0.853 | 0.844 | 0.863 | 0.905 | 0.848 | 0.962 | 0.943 | 0.887 | 1.000 |
| RETN | | | | | | | | | |
| IFIT1, IFI44L, ISG15, HERC6, IL1R2, OLAH, | 0.853 | 0.843 | 0.863 | 0.900 | 0.841 | 0.959 | 0.959 | 0.918 | 1.000 |
| MMP8 | | | | | | | | | |
| RSAD2, OAS1, IFI44L, HERC6, IL1R2, RETN, | 0.853 | 0.843 | 0.863 | 0.895 | 0.828 | 0.961 | 0.947 | 0.895 | 0.998 |
| MMP8 | | | | | | | | | |
| RSAD2, OAS1, IFIT1, IFI44L, ISG15, HERC6, | 0.853 | 0.843 | 0.864 | 0.893 | 0.828 | 0.958 | 0.916 | 0.833 | 1.000 |
| FAM20A | | | | | | | | | |
| RSAD2, IFIT1, IFI44L, HERC6, FAM20A, RETN, | 0.853 | 0.843 | 0.863 | 0.899 | 0.832 | 0.966 | 0.933 | 0.855 | 1 |
| MMP8 | | | | | | | | | |
| RSAD2, OAS1, IFIT1, ISG15, HERC6, SLC1A2, | 0.853 | 0.843 | 0.863 | 0.903 | 0.846 | 0.961 | 0.946 | 0.894 | 0.997 |
| OLAH | | | | | | | | | |
| RSAD2, IFIT1, IFI44L, IL1R2, OLAH, FAM20A, | 0.853 | 0.843 | 0.863 | 0.899 | 0.833 | 0.964 | 0.950 | 0.887 | 1 |
| RETN | | | | | | | | | |
| RSAD2, OAS1, ISG15, HERC6, SLC1A2, OLAH, | 0.853 | 0.843 | 0.863 | 0.903 | 0.845 | 0.960 | 0.940 | 0.885 | 0.996 |
| MMP8 | | | | | | | | | |
| RSAD2, OAS1, IFI44L, HERC6, IL1R2, OLAH, | 0.853 | 0.843 | 0.863 | 0.900 | 0.841 | 0.959 | 0.951 | 0.904 | 0.999 |
| MMP8 | | | | | | | | | |
| RSAD2, IFI27, OAS1, IFIT1, SIGLEC1, ISG15, | 0.853 | 0.843 | 0.863 | 0.888 | 0.820 | 0.955 | 0.917 | 0.843 | 0.992 |
| MMP8 | | | | | | | | | |
| OAS1, IFIT1, IFI44L, OLAH, FAM20A, RETN, | 0.853 | 0.843 | 0.863 | 0.896 | 0.826 | 0.966 | 0.955 | 0.904 | 1 |
| MMP8 | | | | | | | | | |
| RSAD2, IFIT1, ISG15, HERC6, OLAH, RETN, | 0.853 | 0.843 | 0.863 | 0.895 | 0.828 | 0.962 | 0.957 | 0.915 | 0.998 |
| MMP8 | | | | | | | | | |
| RSAD2, IFI44L, SIGLEC1, ISG15, HERC6, | 0.853 | 0.843 | 0.863 | 0.896 | 0.827 | 0.965 | 0.946 | 0.897 | 0.995 |
| RETN, MMP8 | | | | | | | | | |
| RSAD2, IFI44L, SLC1A2, IL1R2, OLAH, RETN, | 0.853 | 0.843 | 0.863 | 0.906 | 0.849 | 0.964 | 0.949 | 0.901 | 0.997 |
| MMP8 | | | | | | | | | |
| RSAD2, IFIT1, IFI44L, SIGLEC1, ISG15, OLAH, | 0.853 | 0.843 | 0.862 | 0.895 | 0.826 | 0.964 | 0.948 | 0.896 | 1.000 |
| RETN | | | | | | | | | |
| OAS1, IFIT1, ISG15, HERC6, IL1R2, OLAH, | 0.853 | 0.843 | 0.863 | 0.900 | 0.842 | 0.959 | 0.960 | 0.920 | 1.000 |
| MMP8 | | | | | | | | | |
| OAS1, IFIT1, IFI44L, SLC1A2, OLAH, RETN, | 0.853 | 0.843 | 0.863 | 0.905 | 0.845 | 0.966 | 0.947 | 0.898 | 0.995 |
| MMP8 | | | | | | | | | |
| OAS1, IFIT1, IFI44L, ISG15, OLAH, FAM20A, | 0.853 | 0.842 | 0.863 | 0.892 | 0.82 | 0.963 | 0.951 | 0.895 | 1 |
| MMP8 | | | | | | | | | |
| RSAD2, IFI27, IFIT1, IFI44L, SIGLEC1, ISG15, | 0.853 | 0.843 | 0.862 | 0.890 | 0.822 | 0.957 | 0.913 | 0.838 | 0.988 |
| MMP8 | | | | | | | | | |
| RSAD2, IFIT1, IFI44L, ISG15, SLC1A2, OLAH, | 0.853 | 0.843 | 0.862 | 0.905 | 0.847 | 0.963 | 0.938 | 0.884 | 0.992 |
| MMP8 | | | | | | | | | |
| OAS1, IFI44L, SIGLEC1, ISG15, OLAH, RETN, | 0.853 | 0.842 | 0.863 | 0.893 | 0.823 | 0.963 | 0.966 | 0.929 | 1 |
| MMP8 | | | | | | | | | |
| RSAD2, IFIT1, IFI44L, ISG15, HERC6, OLAH, | 0.853 | 0.843 | 0.862 | 0.896 | 0.831 | 0.961 | 0.943 | 0.893 | 0.994 |
| RETN | | | | | | | | | |
| RSAD2, IFIT1, IFI44L, ISG15, HERC6, IL1R2, | 0.853 | 0.843 | 0.862 | 0.897 | 0.837 | 0.957 | 0.946 | 0.896 | 0.996 |
| MMP8 | | | | | | | | | |
| RSAD2, IFI44L, HERC6, IL1R2, OLAH, RETN, | 0.853 | 0.842 | 0.863 | 0.900 | 0.836 | 0.963 | 0.959 | 0.918 | 0.999 |
| MMP8 | | | | | | | | | |
| RSAD2, IFI44L, ISG15, SLC1A2, IL1R2, OLAH, | 0.853 | 0.843 | 0.862 | 0.908 | 0.851 | 0.964 | 0.945 | 0.893 | 0.996 |
| RETN | | | | | | | | | |
| RSAD2, IFI27, IFIT1, ISG15, HERC6, SLC1A2, | 0.853 | 0.843 | 0.862 | 0.900 | 0.844 | 0.957 | 0.902 | 0.821 | 0.983 |
| MMP8 | | | | | | | | | |
| OAS1, IFIT1, IFI44L, SLC1A2, IL1R2, OLAH, | 0.853 | 0.842 | 0.863 | 0.910 | 0.854 | 0.966 | 0.942 | 0.891 | 0.994 |
| RETN | | | | | | | | | |
| RSAD2, IFIT1, SLC1A2, IL1R2, OLAH, RETN, | 0.853 | 0.843 | 0.862 | 0.907 | 0.849 | 0.964 | 0.957 | 0.914 | 0.999 |
| MMP8 | | | | | | | | | |
| OAS1, IFIT1, IFI44L, ISG15, HERC6, IL1R2, | 0.853 | 0.843 | 0.862 | 0.898 | 0.839 | 0.957 | 0.948 | 0.899 | 0.997 |
| MMP8 | | | | | | | | | |
| RSAD2, IFIT1, IFI44L, HERC6, SLC1A2, IL1R2, | 0.852 | 0.843 | 0.862 | 0.903 | 0.846 | 0.960 | 0.951 | 0.902 | 1.000 |
| MMP8 | | | | | | | | | |
| RSAD2, IFIT1, IFI44L, ISG15, HERC6, FAM20A, | 0.852 | 0.842 | 0.863 | 0.894 | 0.830 | 0.957 | 0.911 | 0.826 | 0.995 |
| MMP8 | | | | | | | | | |
| IFIT1, ISG15, HERC6, SLC1A2, IL1R2, RETN, | 0.852 | 0.843 | 0.862 | 0.903 | 0.845 | 0.962 | 0.952 | 0.903 | 1 |
| MMP8 | | | | | | | | | |
| RSAD2, OAS1, IFIT1, IFI44L, HERC6, IL1R2, | 0.852 | 0.843 | 0.862 | 0.899 | 0.839 | 0.960 | 0.947 | 0.898 | 0.995 |
| OLAH | | | | | | | | | |
| RSAD2, OAS1, IFIT1, IFI44L, HERC6, SLC1A2, | 0.852 | 0.843 | 0.862 | 0.903 | 0.847 | 0.959 | 0.951 | 0.901 | 1 |
| IL1R2 | | | | | | | | | |
| RSAD2, IFIT1, ISG15, HERC6, IL1R2, OLAH, | 0.852 | 0.842 | 0.862 | 0.900 | 0.842 | 0.959 | 0.959 | 0.918 | 1.000 |
| MMP8 | | | | | | | | | |
| RSAD2, OAS1, IFIT1, IFI44L, ISG15, HERC6, | 0.852 | 0.843 | 0.862 | 0.897 | 0.838 | 0.956 | 0.947 | 0.897 | 0.997 |
| IL1R2 | | | | | | | | | |
| RSAD2, OAS1, IFIT1, HERC6, OLAH, RETN, | 0.852 | 0.842 | 0.863 | 0.890 | 0.820 | 0.960 | 0.955 | 0.913 | 0.998 |
| MMP8 | | | | | | | | | |
| RSAD2, IFIT1, HERC6, SLC1A2, IL1R2, RETN, | 0.852 | 0.843 | 0.862 | 0.903 | 0.844 | 0.962 | 0.952 | 0.904 | 1.000 |
| MMP8 | | | | | | | | | |
| OAS1, IFIT1, SIGLEC1, ISG15, HERC6, | 0.852 | 0.843 | 0.862 | 0.896 | 0.837 | 0.955 | 0.924 | 0.861 | 0.987 |
| SLC1A2, MMP8 | | | | | | | | | |
| RSAD2, OAS1, IFI44L, SIGLEC1, HERC6, | 0.852 | 0.842 | 0.862 | 0.890 | 0.819 | 0.962 | 0.946 | 0.896 | 0.995 |
| RETN, MMP8 | | | | | | | | | |
| OAS1, IFI44L, SLC1A2, IL1R2, OLAH, RETN, | 0.852 | 0.842 | 0.862 | 0.907 | 0.849 | 0.964 | 0.945 | 0.894 | 0.995 |
| MMP8 | | | | | | | | | |
| IFIT1, IFI44L, ISG15, HERC6, SLC1A2, IL1R2, | 0.852 | 0.843 | 0.862 | 0.901 | 0.844 | 0.958 | 0.952 | 0.904 | 1 |
| MMP8 | | | | | | | | | |
| RSAD2, OAS1, IFI44L, SLC1A2, IL1R2, OLAH, | 0.852 | 0.842 | 0.863 | 0.907 | 0.850 | 0.963 | 0.939 | 0.884 | 0.994 |
| RETN | | | | | | | | | |
| RSAD2, IFI44L, SIGLEC1, HERC6, SLC1A2, | 0.852 | 0.843 | 0.862 | 0.903 | 0.840 | 0.965 | 0.939 | 0.885 | 0.993 |
| RETN, MMP8 | | | | | | | | | |
| RSAD2, OAS1, IFI44L, SLC1A2, IL1R2, OLAH, | 0.852 | 0.842 | 0.862 | 0.905 | 0.849 | 0.961 | 0.941 | 0.888 | 0.995 |
| MMP8 | | | | | | | | | |
| RSAD2, OAS1, IFIT1, IFI44L, OLAH, FAM20A, | 0.852 | 0.842 | 0.862 | 0.896 | 0.826 | 0.965 | 0.943 | 0.885 | 1 |
| RETN | | | | | | | | | |
| OAS1, IFI44L, HERC6, SLC1A2, FAM20A, | 0.852 | 0.842 | 0.863 | 0.901 | 0.837 | 0.965 | 0.936 | 0.857 | 1 |
| RETN, MMP8 | | | | | | | | | |
| IFI44L, ISG15, HERC6, SLC1A2, IL1R2, RETN, | 0.852 | 0.843 | 0.862 | 0.905 | 0.849 | 0.962 | 0.946 | 0.890 | 1 |
| MMP8 | | | | | | | | | |
| OAS1, IFIT1, IFI44L, HERC6, IL1R2, OLAH, | 0.852 | 0.842 | 0.862 | 0.898 | 0.834 | 0.963 | 0.947 | 0.897 | 0.996 |
| RETN | | | | | | | | | |
| RSAD2, IFI44L, ISG15, SLC1A2, IL1R2, OLAH, | 0.852 | 0.843 | 0.862 | 0.904 | 0.847 | 0.961 | 0.945 | 0.892 | 0.998 |
| MMP8 | | | | | | | | | |
| RSAD2, OAS1, IFIT1, IFI44L, HERC6, OLAH, | 0.852 | 0.842 | 0.862 | 0.892 | 0.824 | 0.961 | 0.938 | 0.884 | 0.992 |
| RETN | | | | | | | | | |
| RSAD2, IFIT1, IFI44L, ISG15, SLC1A2, IL1R2, | 0.852 | 0.843 | 0.862 | 0.909 | 0.852 | 0.965 | 0.945 | 0.893 | 0.996 |
| OLAH | | | | | | | | | |
| OAS1, IFI44L, ISG15, SLC1A2, OLAH, RETN, | 0.852 | 0.842 | 0.862 | 0.906 | 0.845 | 0.966 | 0.943 | 0.893 | 0.994 |
| MMP8 | | | | | | | | | |
| IFIT1, IFI44L, ISG15, HERC6, SLC1A2, | 0.852 | 0.842 | 0.862 | 0.899 | 0.840 | 0.958 | 0.913 | 0.823 | 1 |
| FAM20A, MMP8 | | | | | | | | | |
| OAS1, IFIT1, IFI44L, SIGLEC1, HERC6, | 0.852 | 0.843 | 0.861 | 0.893 | 0.833 | 0.953 | 0.912 | 0.843 | 0.981 |
| SLC1A2, MMP8 | | | | | | | | | |
| RSAD2, OAS1, IFI44L, HERC6, SLC1A2, IL1R2, | 0.852 | 0.842 | 0.862 | 0.901 | 0.843 | 0.960 | 0.941 | 0.880 | 1 |
| RETN | | | | | | | | | |
| RSAD2, OAS1, IFIT1, SIGLEC1, HERC6, | 0.852 | 0.843 | 0.861 | 0.895 | 0.835 | 0.955 | 0.918 | 0.852 | 0.985 |
| SLC1A2, MMP8 | | | | | | | | | |
| RSAD2, OAS1, ISG15, SLC1A2, IL1R2, OLAH, | 0.852 | 0.842 | 0.862 | 0.906 | 0.849 | 0.963 | 0.940 | 0.884 | 0.997 |
| RETN | | | | | | | | | |
| OAS1, IFI44L, ISG15, SLC1A2, IL1R2, OLAH, | 0.852 | 0.842 | 0.862 | 0.905 | 0.849 | 0.962 | 0.941 | 0.888 | 0.995 |
| MMP8 | | | | | | | | | |
| OAS1, IFI44L, ISG15, SLC1A2, IL1R2, OLAH, | 0.852 | 0.842 | 0.862 | 0.909 | 0.853 | 0.966 | 0.942 | 0.890 | 0.995 |
| RETN | | | | | | | | | |
| RSAD2, OAS1, IFIT1, IFI44L, SLC1A2, IL1R2, | 0.852 | 0.842 | 0.862 | 0.909 | 0.852 | 0.965 | 0.946 | 0.895 | 0.996 |
| OLAH | | | | | | | | | |
| RSAD2, OAS1, IFIT1, IFI44L, SLC1A2, OLAH, | 0.852 | 0.842 | 0.862 | 0.909 | 0.851 | 0.967 | 0.937 | 0.883 | 0.991 |
| RETN | | | | | | | | | |
| RSAD2, OAS1, IFI44L, HERC6, OLAH, RETN, | 0.852 | 0.842 | 0.862 | 0.893 | 0.825 | 0.962 | 0.948 | 0.900 | 0.995 |
| MMP8 | | | | | | | | | |
| RSAD2, OAS1, ISG15, SLC1A2, IL1R2, OLAH, | 0.852 | 0.843 | 0.861 | 0.903 | 0.846 | 0.961 | 0.939 | 0.882 | 0.996 |
| MMP8 | | | | | | | | | |
| RSAD2, OAS1, IFI44L, ISG15, HERC6, IL1R2, | 0.852 | 0.842 | 0.862 | 0.900 | 0.841 | 0.959 | 0.947 | 0.898 | 0.995 |
| OLAH | | | | | | | | | |
| RSAD2, IFIT1, IFI44L, SIGLEC1, ISG15, OLAH, | 0.852 | 0.842 | 0.862 | 0.894 | 0.827 | 0.961 | 0.951 | 0.903 | 0.999 |
| MMP8 | | | | | | | | | |
| RSAD2, IFIT1, IFI44L, SLC1A2, IL1R2, OLAH, | 0.852 | 0.842 | 0.862 | 0.908 | 0.852 | 0.965 | 0.945 | 0.894 | 0.995 |
| RETN | | | | | | | | | |
| RSAD2, IFIT1, IFI44L, SLC1A2, OLAH, RETN, | 0.852 | 0.842 | 0.862 | 0.905 | 0.844 | 0.966 | 0.947 | 0.898 | 0.995 |
| MMP8 | | | | | | | | | |
| RSAD2, IFIT1, ISG15, HERC6, SLC1A2, OLAH, | 0.852 | 0.842 | 0.862 | 0.902 | 0.844 | 0.960 | 0.943 | 0.891 | 0.996 |
| MMP8 | | | | | | | | | |
| RSAD2, IFI44L, ISG15, SLC1A2, OLAH, RETN, | 0.852 | 0.842 | 0.862 | 0.907 | 0.847 | 0.967 | 0.945 | 0.895 | 0.995 |
| MMP8 | | | | | | | | | |
| OAS1, IFI44L, HERC6, SLC1A2, IL1R2, RETN, | 0.852 | 0.842 | 0.862 | 0.903 | 0.845 | 0.961 | 0.943 | 0.885 | 1 |
| MMP8 | | | | | | | | | |
| RSAD2, OAS1, SLC1A2, IL1R2, OLAH, RETN, | 0.852 | 0.842 | 0.862 | 0.906 | 0.849 | 0.964 | 0.942 | 0.888 | 0.996 |
| MMP8 | | | | | | | | | |
| RSAD2, IFI44L, ISG15, HERC6, OLAH, RETN, | 0.852 | 0.841 | 0.862 | 0.899 | 0.833 | 0.965 | 0.954 | 0.911 | 0.998 |
| MMP8 | | | | | | | | | |
| OAS1, IFIT1, ISG15, OLAH, FAM20A, RETN, | 0.852 | 0.841 | 0.862 | 0.891 | 0.820 | 0.962 | 0.959 | 0.905 | 1 |
| MMP8 | | | | | | | | | |
| RSAD2, IFIT1, ISG15, SLC1A2, IL1R2, OLAH, | 0.852 | 0.842 | 0.861 | 0.907 | 0.849 | 0.965 | 0.946 | 0.895 | 0.996 |
| RETN | | | | | | | | | |
| RSAD2, ISG15, HERC6, SLC1A2, IL1R2, RETN, | 0.852 | 0.842 | 0.861 | 0.901 | 0.842 | 0.959 | 0.941 | 0.880 | 1 |
| MMP8 | | | | | | | | | |
| RSAD2, IFIT1, IFI44L, ISG15, SLC1A2, OLAH, | 0.852 | 0.842 | 0.861 | 0.909 | 0.851 | 0.967 | 0.934 | 0.877 | 0.990 |
| RETN | | | | | | | | | |
| RSAD2, IFI27, OAS1, IFIT1, ISG15, SLC1A2, | 0.852 | 0.841 | 0.862 | 0.892 | 0.824 | 0.960 | 0.938 | 0.858 | 1 |
| FAM20A | | | | | | | | | |
| OAS1, IFIT1, IFI44L, ISG15, OLAH, FAM20A, | 0.852 | 0.841 | 0.862 | 0.889 | 0.817 | 0.961 | 0.952 | 0.898 | 1 |
| RETN | | | | | | | | | |
| RSAD2, OAS1, IFI44L, ISG15, SLC1A2, IL1R2, | 0.852 | 0.841 | 0.862 | 0.906 | 0.850 | 0.963 | 0.940 | 0.885 | 0.995 |
| OLAH | | | | | | | | | |
| RSAD2, OAS1, IFI44L, ISG15, HERC6, | 0.852 | 0.841 | 0.862 | 0.891 | 0.826 | 0.957 | 0.925 | 0.845 | 1 |
| FAM20A, MMP8 | | | | | | | | | |
| RSAD2, OAS1, IFI44L, ISG15, SLC1A2, OLAH, | 0.852 | 0.842 | 0.862 | 0.905 | 0.847 | 0.963 | 0.933 | 0.875 | 0.990 |
| MMP8 | | | | | | | | | |
| RSAD2, OAS1, IFI44L, ISG15, HERC6, SLC1A2, | 0.852 | 0.842 | 0.861 | 0.900 | 0.845 | 0.955 | 0.945 | 0.885 | 1 |
| IL1R2 | | | | | | | | | |
| RSAD2, IFI27, IFI44L, SIGLEC1, ISG15, HERC6, | 0.851 | 0.843 | 0.860 | 0.898 | 0.842 | 0.954 | 0.940 | 0.885 | 0.995 |
| SLC1A2 | | | | | | | | | |
| RSAD2, OAS1, HERC6, SLC1A2, IL1R2, RETN, | 0.851 | 0.842 | 0.861 | 0.902 | 0.844 | 0.959 | 0.939 | 0.872 | 1 |
| MMP8 | | | | | | | | | |
| OAS1, IFI44L, ISG15, HERC6, IL1R2, OLAH, | 0.851 | 0.842 | 0.861 | 0.899 | 0.841 | 0.958 | 0.954 | 0.910 | 0.998 |
| MMP8 | | | | | | | | | |
| RSAD2, IFIT1, IFI44L, HERC6, IL1R2, OLAH, | 0.851 | 0.842 | 0.861 | 0.899 | 0.839 | 0.959 | 0.955 | 0.912 | 0.999 |
| MMP8 | | | | | | | | | |
| RSAD2, OAS1, ISG15, HERC6, SLC1A2, IL1R2, | 0.851 | 0.842 | 0.861 | 0.899 | 0.842 | 0.955 | 0.936 | 0.869 | 1 |
| MMP8 | | | | | | | | | |
| RSAD2, OAS1, IFI44L, ISG15, SLC1A2, OLAH, | 0.851 | 0.841 | 0.862 | 0.909 | 0.851 | 0.967 | 0.935 | 0.879 | 0.991 |
| RETN | | | | | | | | | |
| RSAD2, OAS1, IFI44L, SLC1A2, OLAH, RETN, | 0.851 | 0.841 | 0.861 | 0.906 | 0.846 | 0.966 | 0.943 | 0.893 | 0.994 |
| MMP8 | | | | | | | | | |
| OAS1, IFI44L, ISG15, HERC6, SLC1A2, IL1R2, | 0.851 | 0.841 | 0.861 | 0.900 | 0.845 | 0.956 | 0.939 | 0.879 | 0.999 |
| MMP8 | | | | | | | | | |
| RSAD2, IFI27, IFIT1, SIGLEC1, ISG15, HERC6, | 0.851 | 0.842 | 0.860 | 0.901 | 0.846 | 0.956 | 0.941 | 0.888 | 0.995 |
| SLC1A2 | | | | | | | | | |
| RSAD2, IFIT1, IFI44L, HERC6, SLC1A2, IL1R2, | 0.851 | 0.842 | 0.861 | 0.901 | 0.842 | 0.960 | 0.939 | 0.881 | 0.998 |
| RETN | | | | | | | | | |
| RSAD2, ISG15, SLC1A2, IL1R2, OLAH, RETN, | 0.851 | 0.842 | 0.861 | 0.905 | 0.848 | 0.963 | 0.943 | 0.888 | 0.999 |
| MMP8 | | | | | | | | | |
| RSAD2, OAS1, IFI44L, ISG15, HERC6, IL1R2, | 0.851 | 0.841 | 0.861 | 0.895 | 0.837 | 0.954 | 0.946 | 0.895 | 0.996 |
| MMP8 | | | | | | | | | |
| RSAD2, OAS1, IFI44L, ISG15, HERC6, SLC1A2, | 0.851 | 0.841 | 0.862 | 0.898 | 0.835 | 0.961 | 0.933 | 0.870 | 0.995 |
| RETN | | | | | | | | | |
| RSAD2, OAS1, IFIT1, IFI44L, ISG15, HERC6, | 0.851 | 0.841 | 0.861 | 0.892 | 0.825 | 0.959 | 0.926 | 0.864 | 0.988 |
| RETN | | | | | | | | | |
| RSAD2, OAS1, IFIT1, IFI44L, ISG15, SLC1A2, | 0.851 | 0.841 | 0.861 | 0.909 | 0.852 | 0.966 | 0.935 | 0.880 | 0.990 |
| OLAH | | | | | | | | | |
| OAS1, IFIT1, IFI44L, ISG15, HERC6, IL1R2, | 0.851 | 0.841 | 0.861 | 0.899 | 0.840 | 0.959 | 0.952 | 0.907 | 0.997 |
| OLAH | | | | | | | | | |
| RSAD2, IFIT1, IFI44L, ISG15, HERC6, IL1R2, | 0.851 | 0.841 | 0.861 | 0.900 | 0.840 | 0.960 | 0.948 | 0.900 | 0.996 |
| OLAH | | | | | | | | | |
| RSAD2, IFIT1, IFI44L, HERC6, OLAH, RETN, | 0.851 | 0.841 | 0.861 | 0.894 | 0.825 | 0.962 | 0.952 | 0.907 | 0.997 |
| MMP8 | | | | | | | | | |
| OAS1, IFI44L, ISG15, OLAH, FAM20A, RETN, | 0.851 | 0.840 | 0.861 | 0.895 | 0.825 | 0.966 | 0.957 | 0.905 | 1 |
| MMP8 | | | | | | | | | |
| RSAD2, IFI44L, ISG15, HERC6, IL1R2, OLAH, | 0.851 | 0.841 | 0.861 | 0.901 | 0.843 | 0.959 | 0.950 | 0.903 | 0.997 |
| MMP8 | | | | | | | | | |
| RSAD2, IFI27, OAS1, IFI44L, SIGLEC1, ISG15, | 0.851 | 0.842 | 0.860 | 0.896 | 0.837 | 0.956 | 0.952 | 0.902 | 1 |
| SLC1A2 | | | | | | | | | |
| OAS1, IFIT1, ISG15, SLC1A2, OLAH, RETN, | 0.851 | 0.840 | 0.861 | 0.900 | 0.839 | 0.961 | 0.954 | 0.911 | 0.998 |
| MMP8 | | | | | | | | | |
| IFI27, OAS1, IFIT1, IFI44L, SIGLEC1, ISG15, | 0.851 | 0.841 | 0.860 | 0.896 | 0.836 | 0.955 | 0.954 | 0.905 | 1 |
| SLC1A2 | | | | | | | | | |
| RSAD2, OAS1, IFI44L, SIGLEC1, ISG15, OLAH, | 0.851 | 0.840 | 0.861 | 0.891 | 0.821 | 0.962 | 0.950 | 0.901 | 0.999 |
| RETN | | | | | | | | | |
| RSAD2, OAS1, IFIT1, ISG15, SLC1A2, OLAH, | 0.851 | 0.841 | 0.861 | 0.902 | 0.844 | 0.961 | 0.942 | 0.891 | 0.994 |
| MMP8 | | | | | | | | | |
| IFIT1, IFI44L, ISG15, OLAH, FAM20A, RETN, | 0.851 | 0.840 | 0.861 | 0.896 | 0.828 | 0.964 | 0.962 | 0.920 | 1 |
| MMP8 | | | | | | | | | |
| RSAD2, IFI44L, ISG15, HERC6, SLC1A2, IL1R2, | 0.851 | 0.841 | 0.860 | 0.901 | 0.846 | 0.957 | 0.940 | 0.882 | 0.999 |
| MMP8 | | | | | | | | | |
| RSAD2, OAS1, IFIT1, HERC6, SLC1A2, | 0.850 | 0.840 | 0.861 | 0.896 | 0.831 | 0.961 | 0.935 | 0.854 | 1 |
| FAM20A, RETN | | | | | | | | | |
| RSAD2, IFIT1, IFI44L, HERC6, IL1R2, OLAH, | 0.850 | 0.841 | 0.860 | 0.898 | 0.834 | 0.963 | 0.947 | 0.898 | 0.996 |
| RETN | | | | | | | | | |
| RSAD2, OAS1, IFIT1, ISG15, SLC1A2, OLAH, | 0.850 | 0.840 | 0.860 | 0.906 | 0.847 | 0.964 | 0.945 | 0.894 | 0.995 |
| RETN | | | | | | | | | |
| RSAD2, OAS1, IFIT1, IFI44L, IL1R2, OLAH, | 0.850 | 0.840 | 0.860 | 0.895 | 0.832 | 0.958 | 0.955 | 0.913 | 0.998 |
| MMP8 | | | | | | | | | |
| OAS1, IFIT1, IFI44L, ISG15, HERC6, SLC1A2, | 0.850 | 0.840 | 0.860 | 0.898 | 0.836 | 0.960 | 0.921 | 0.851 | 0.990 |
| RETN | | | | | | | | | |
| RSAD2, IFI27, IFIT1, IFI44L, SIGLEC1, HERC6, | 0.850 | 0.841 | 0.859 | 0.899 | 0.844 | 0.955 | 0.940 | 0.885 | 0.995 |
| SLC1A2 | | | | | | | | | |
| RSAD2, IFIT1, IFI44L, SIGLEC1, HERC6, | 0.850 | 0.841 | 0.859 | 0.896 | 0.837 | 0.956 | 0.908 | 0.836 | 0.979 |
| SLC1A2, MMP8 | | | | | | | | | |
| RSAD2, OAS1, IFI44L, SIGLEC1, ISG15, OLAH, | 0.850 | 0.840 | 0.860 | 0.893 | 0.825 | 0.960 | 0.949 | 0.901 | 0.997 |
| MMP8 | | | | | | | | | |

Performance obtained for the identification of the viral or bacterial nature of the infection on the basis of the eight-gene combinations:

TABLE 12

| | AUC | | | AUC | CI 95% | | AUC | CI 95% | |
|---|---|---|---|---|---|---|---|---|---|
| Model | model | CI 95% | | Train | Train | | Test | Test | |
| IFI27, IFI44L, SIGLEC1, ISG15, | 0.899 | 0.891 | 0.907 | 0.935 | 0.885 | 0.985 | 0.952 | 0.892 | 1 |
| IL1R2, OLAH, FAM20A, MMP8 | | | | | | | | | |
| IFI27, IFIT1, SIGLEC1, HERC6, | 0.899 | 0.891 | 0.908 | 0.935 | 0.885 | 0.986 | 0.954 | 0.893 | 1 |
| IL1R2, OLAH, FAM20A, MMP8 | | | | | | | | | |
| RSAD2, IFI27, SIGLEC1, HERC6, | 0.899 | 0.890 | 0.908 | 0.935 | 0.885 | 0.986 | 0.951 | 0.891 | 1 |
| IL1R2, OLAH, FAM20A, MMP8 | | | | | | | | | |
| IFI27, OAS1, IFIT1, SIGLEC1, | 0.899 | 0.890 | 0.908 | 0.933 | 0.880 | 0.986 | 0.945 | 0.881 | 1 |
| HERC6, OLAH, FAM20A, MMP8 | | | | | | | | | |
| IFI27, OAS1, SIGLEC1, HERC6, | 0.899 | 0.890 | 0.908 | 0.936 | 0.885 | 0.986 | 0.950 | 0.890 | 1 |
| IL1R2, OLAH, FAM20A, MMP8 | | | | | | | | | |
| IFI27, IFIT1, SIGLEC1, ISG15, | 0.899 | 0.890 | 0.907 | 0.937 | 0.886 | 0.987 | 0.952 | 0.889 | 1 |
| IL1R2, OLAH, FAM20A, MMP8 | | | | | | | | | |
| IFI27, OAS1, IFIT1, SIGLEC1, | 0.899 | 0.890 | 0.907 | 0.935 | 0.885 | 0.986 | 0.955 | 0.899 | 1 |
| IL1R2, OLAH, FAM20A, MMP8 | | | | | | | | | |
| IFI27, IFIT1, IFI44L, SIGLEC1, | 0.898 | 0.890 | 0.906 | 0.936 | 0.886 | 0.986 | 0.955 | 0.895 | 1 |
| IL1R2, OLAH, FAM20A, MMP8 | | | | | | | | | |
| IFI27, OAS1, IFIT1, SIGLEC1, | 0.898 | 0.890 | 0.907 | 0.933 | 0.883 | 0.983 | 0.951 | 0.885 | 1 |
| HERC6, IL1R2, FAM20A, MMP8 | | | | | | | | | |
| IFI27, OAS1, IFIT1, IFI44L, | 0.898 | 0.889 | 0.907 | 0.932 | 0.879 | 0.985 | 0.940 | 0.876 | 1 |
| SIGLEC1, HERC6, OLAH, FAM20A | | | | | | | | | |
| IFI27, SIGLEC1, ISG15, HERC6, | 0.898 | 0.889 | 0.907 | 0.933 | 0.881 | 0.986 | 0.950 | 0.886 | 1 |
| IL1R2, OLAH, FAM20A, MMP8 | | | | | | | | | |
| IFI27, IFIT1, SIGLEC1, ISG15, | 0.898 | 0.888 | 0.907 | 0.931 | 0.877 | 0.986 | 0.951 | 0.892 | 1 |
| HERC6, OLAH, FAM20A, MMP8 | | | | | | | | | |
| IFI27, IFI44L, SIGLEC1, HERC6, | 0.898 | 0.889 | 0.906 | 0.936 | 0.886 | 0.985 | 0.953 | 0.892 | 1 |
| IL1R2, OLAH, FAM20A, MMP8 | | | | | | | | | |
| RSAD2, IFI27, OAS1, SIGLEC1, | 0.898 | 0.888 | 0.907 | 0.934 | 0.881 | 0.986 | 0.947 | 0.887 | 1 |
| HERC6, OLAH, FAM20A, MMP8 | | | | | | | | | |
| IFI27, IFIT1, IFI44L, SIGLEC1, | 0.897 | 0.889 | 0.906 | 0.933 | 0.882 | 0.984 | 0.952 | 0.885 | 1 |
| HERC6, IL1R2, FAM20A, MMP8 | | | | | | | | | |
| IFI27, IFIT1, IFI44L, SIGLEC1, | 0.897 | 0.889 | 0.906 | 0.931 | 0.877 | 0.986 | 0.955 | 0.896 | 1 |
| HERC6, OLAH, FAM20A, MMP8 | | | | | | | | | |
| RSAD2, IFI27, IFIT1, SIGLEC1, | 0.897 | 0.888 | 0.906 | 0.934 | 0.882 | 0.986 | 0.948 | 0.887 | 1 |
| HERC6, OLAH, FAM20A, MMP8 | | | | | | | | | |
| RSAD2, IFI27, IFIT1, SIGLEC1, | 0.897 | 0.889 | 0.906 | 0.935 | 0.884 | 0.986 | 0.954 | 0.897 | 1 |
| IL1R2, OLAH, FAM20A, MMP8 | | | | | | | | | |
| RSAD2, IFI27, OAS1, SIGLEC1, | 0.897 | 0.888 | 0.906 | 0.935 | 0.884 | 0.986 | 0.952 | 0.892 | 1 |
| IL1R2, OLAH, FAM20A, MMP8 | | | | | | | | | |
| RSAD2, IFI27, IFIT1, SIGLEC1, | 0.897 | 0.889 | 0.905 | 0.933 | 0.883 | 0.983 | 0.951 | 0.884 | 1 |
| HERC6, IL1R2, FAM20A, MMP8 | | | | | | | | | |
| IFI27, IFIT1, IFI44L, SIGLEC1, | 0.897 | 0.888 | 0.905 | 0.932 | 0.879 | 0.985 | 0.945 | 0.881 | 1 |
| HERC6, IL1R2, OLAH, FAM20A | | | | | | | | | |
| IFI27, IFIT1, IFI44L, SIGLEC1, | 0.897 | 0.888 | 0.906 | 0.932 | 0.878 | 0.986 | 0.941 | 0.878 | 1 |
| ISG15, HERC6, OLAH, FAM20A | | | | | | | | | |
| IFI27, OAS1, SIGLEC1, ISG15, | 0.897 | 0.888 | 0.906 | 0.936 | 0.884 | 0.987 | 0.950 | 0.886 | 1 |
| IL1R2, OLAH, FAM20A, MMP8 | | | | | | | | | |
| IFI27, IFIT1, SIGLEC1, ISG15, | 0.897 | 0.888 | 0.905 | 0.932 | 0.880 | 0.983 | 0.953 | 0.885 | 1 |
| HERC6, IL1R2, FAM20A, MMP8 | | | | | | | | | |
| IFI27, OAS1, IFIT1, SIGLEC1, | 0.896 | 0.888 | 0.905 | 0.932 | 0.880 | 0.984 | 0.941 | 0.878 | 1 |
| HERC6, IL1R2, OLAH, FAM20A | | | | | | | | | |
| IFI27, OAS1, SIGLEC1, HERC6, | 0.896 | 0.886 | 0.906 | 0.933 | 0.877 | 0.988 | 0.948 | 0.886 | 1 |
| OLAH, FAM20A, RETN, MMP8 | | | | | | | | | |
| IFI27, OAS1, IFI44L, SIGLEC1, | 0.896 | 0.888 | 0.905 | 0.934 | 0.884 | 0.983 | 0.948 | 0.881 | 1 |
| HERC6, IL1R2, FAM20A, MMP8 | | | | | | | | | |
| IFI27, IFI44L, SIGLEC1, ISG15, | 0.896 | 0.888 | 0.904 | 0.933 | 0.882 | 0.983 | 0.950 | 0.882 | 1 |
| HERC6, IL1R2, FAM20A, MMP8 | | | | | | | | | |
| IFI27, OAS1, IFIT1, SIGLEC1, | 0.896 | 0.887 | 0.905 | 0.933 | 0.880 | 0.985 | 0.938 | 0.873 | 1 |
| ISG15, HERC6, OLAH, FAM20A | | | | | | | | | |
| IFI27, OAS1, SIGLEC1, HERC6, | 0.896 | 0.887 | 0.905 | 0.934 | 0.881 | 0.987 | 0.950 | 0.882 | 1 |
| IL1R2, FAM20A, RETN, MMP8 | | | | | | | | | |
| IFI27, OAS1, IFIT1, SIGLEC1, | 0.896 | 0.887 | 0.905 | 0.933 | 0.880 | 0.985 | 0.939 | 0.874 | 1 |
| HERC6, OLAH, FAM20A, RETN | | | | | | | | | |
| RSAD2, IFI27, OAS1, IFIT1, | 0.896 | 0.887 | 0.905 | 0.931 | 0.878 | 0.985 | 0.939 | 0.875 | 1 |
| SIGLEC1, HERC6, OLAH, FAM20A | | | | | | | | | |
| IFI27, IFIT1, SIGLEC1, HERC6, | 0.896 | 0.887 | 0.905 | 0.936 | 0.885 | 0.988 | 0.955 | 0.888 | 1 |
| IL1R2, FAM20A, RETN, MMP8 | | | | | | | | | |
| RSAD2, IFI27, SIGLEC1, ISG15, | 0.896 | 0.887 | 0.905 | 0.936 | 0.885 | 0.987 | 0.952 | 0.892 | 1 |
| IL1R2, OLAH, FAM20A, MMP8 | | | | | | | | | |
| IFI27, OAS1, IFIT1, SIGLEC1, | 0.896 | 0.887 | 0.905 | 0.928 | 0.870 | 0.986 | 0.953 | 0.898 | 1 |
| ISG15, OLAH, FAM20A, MMP8 | | | | | | | | | |
| RSAD2, IFI27, SIGLEC1, HERC6, | 0.896 | 0.886 | 0.905 | 0.935 | 0.882 | 0.989 | 0.951 | 0.892 | 1 |
| OLAH, FAM20A, RETN, MMP8 | | | | | | | | | |
| IFI27, IFIT1, SIGLEC1, HERC6, | 0.896 | 0.886 | 0.905 | 0.933 | 0.878 | 0.988 | 0.957 | 0.898 | 1 |
| OLAH, FAM20A, RETN, MMP8 | | | | | | | | | |
| IFI27, SIGLEC1, ISG15, IL1R2, | 0.896 | 0.886 | 0.905 | 0.933 | 0.879 | 0.987 | 0.953 | 0.891 | 1 |
| OLAH, FAM20A, RETN, MMP8 | | | | | | | | | |
| RSAD2, IFI27, SIGLEC1, ISG15, | 0.895 | 0.886 | 0.905 | 0.934 | 0.881 | 0.988 | 0.947 | 0.887 | 1 |
| HERC6, OLAH, FAM20A, MMP8 | | | | | | | | | |
| IFI27, OAS1, SIGLEC1, ISG15, | 0.895 | 0.886 | 0.905 | 0.929 | 0.873 | 0.986 | 0.946 | 0.884 | 1 |
| HERC6, OLAH, FAM20A, MMP8 | | | | | | | | | |
| IFI27, IFIT1, IFI44L, SIGLEC1, | 0.895 | 0.887 | 0.904 | 0.930 | 0.875 | 0.985 | 0.942 | 0.879 | 1 |
| HERC6, OLAH, FAM20A, RETN | | | | | | | | | |
| RSAD2, IFI27, IFIT1, IFI44L, | 0.895 | 0.887 | 0.904 | 0.932 | 0.879 | 0.985 | 0.940 | 0.877 | 1 |
| SIGLEC1, HERC6, OLAH, FAM20A | | | | | | | | | |
| RSAD2, IFI27, IFI44L, SIGLEC1, | 0.895 | 0.887 | 0.904 | 0.933 | 0.883 | 0.983 | 0.950 | 0.880 | 1 |
| HERC6, IL1R2, FAM20A, MMP8 | | | | | | | | | |
| RSAD2, IFI27, OAS1, SIGLEC1, | 0.895 | 0.886 | 0.904 | 0.935 | 0.884 | 0.985 | 0.949 | 0.881 | 1 |
| HERC6, IL1R2, FAM20A, MMP8 | | | | | | | | | |
| IFI27, IFIT1, IFI44L, SIGLEC1, | 0.895 | 0.886 | 0.904 | 0.930 | 0.874 | 0.986 | 0.95 | 0.894 | 1 |
| ISG15, OLAH, FAM20A, MMP8 | | | | | | | | | |
| IFI27, IFIT1, SIGLEC1, ISG15, | 0.895 | 0.887 | 0.904 | 0.933 | 0.881 | 0.986 | 0.941 | 0.877 | 1 |
| HERC6, IL1R2, OLAH, FAM20A | | | | | | | | | |
| IFI27, OAS1, IFIT1, IFI44L, | 0.895 | 0.887 | 0.903 | 0.931 | 0.880 | 0.982 | 0.957 | 0.896 | 1 |
| SIGLEC1, IL1R2, FAM20A, MMP8 | | | | | | | | | |
| RSAD2, IFI27, OAS1, HERC6, | 0.895 | 0.886 | 0.904 | 0.933 | 0.884 | 0.983 | 0.927 | 0.850 | 1 |
| IL1R2, OLAH, FAM20A, MMP8 | | | | | | | | | |
| RSAD2, IFI27, SIGLEC1, HERC6, | 0.895 | 0.886 | 0.904 | 0.935 | 0.883 | 0.987 | 0.949 | 0.880 | 1 |
| IL1R2, FAM20A, RETN, MMP8 | | | | | | | | | |
| RSAD2, IFI27, IFIT1, SIGLEC1, | 0.895 | 0.887 | 0.903 | 0.934 | 0.883 | 0.986 | 0.939 | 0.874 | 1 |
| HERC6, IL1R2, OLAH, FAM20A | | | | | | | | | |
| IFI27, IFI44L, SIGLEC1, ISG15, | 0.895 | 0.886 | 0.904 | 0.928 | 0.871 | 0.985 | 0.945 | 0.881 | 1 |
| HERC6, OLAH, FAM20A, MMP8 | | | | | | | | | |
| IFI27, IFIT1, SIGLEC1, ISG15, | 0.895 | 0.886 | 0.904 | 0.931 | 0.876 | 0.987 | 0.942 | 0.879 | 1 |
| HERC6, OLAH, FAM20A, RETN | | | | | | | | | |
| IFI27, IFIT1, IFI44L, SIGLEC1, | 0.895 | 0.886 | 0.903 | 0.934 | 0.882 | 0.986 | 0.953 | 0.888 | 1 |
| IL1R2, FAM20A, RETN, MMP8 | | | | | | | | | |
| IFI27, IFIT1, IFI44L, SIGLEC1, | 0.895 | 0.887 | 0.903 | 0.932 | 0.881 | 0.983 | 0.952 | 0.885 | 1 |
| ISG15, IL1R2, FAM20A, MMP8 | | | | | | | | | |
| IFI27, OAS1, IFI44L, SIGLEC1, | 0.895 | 0.886 | 0.904 | 0.935 | 0.885 | 0.985 | 0.954 | 0.896 | 1 |
| IL1R2, OLAH, FAM20A, MMP8 | | | | | | | | | |
| IFI27, IFI44L, SIGLEC1, ISG15, | 0.895 | 0.886 | 0.903 | 0.930 | 0.876 | 0.983 | 0.942 | 0.877 | 1 |
| HERC6, IL1R2, OLAH, FAM20A | | | | | | | | | |
| IFI27, OAS1, IFIT1, IFI44L, | 0.895 | 0.887 | 0.903 | 0.934 | 0.884 | 0.984 | 0.946 | 0.879 | 1 |
| SIGLEC1, HERC6, IL1R2, FAM20A | | | | | | | | | |
| RSAD2, IFI27, OAS1, SIGLEC1, | 0.895 | 0.886 | 0.904 | 0.931 | 0.878 | 0.984 | 0.940 | 0.874 | 1 |
| HERC6, IL1R2, OLAH, FAM20A | | | | | | | | | |
| IFI27, IFIT1, SIGLEC1, IL1R2, | 0.895 | 0.886 | 0.903 | 0.934 | 0.881 | 0.988 | 0.958 | 0.899 | 1 |
| OLAH, FAM20A, RETN, MMP8 | | | | | | | | | |
| IFI27, OAS1, IFIT1, SIGLEC1, | 0.895 | 0.885 | 0.904 | 0.933 | 0.879 | 0.987 | 0.942 | 0.873 | 1 |
| HERC6, FAM20A, RETN, MMP8 | | | | | | | | | |
| IFI27, OAS1, IFIT1, IFI44L, | 0.894 | 0.885 | 0.904 | 0.927 | 0.869 | 0.985 | 0.957 | 0.902 | 1 |
| SIGLEC1, OLAH, FAM20A, MMP8 | | | | | | | | | |
| IFI27, OAS1, IFIT1, SIGLEC1, | 0.894 | 0.886 | 0.903 | 0.934 | 0.882 | 0.986 | 0.954 | 0.891 | 1 |
| IL1R2, FAM20A, RETN, MMP8 | | | | | | | | | |
| IFI27, IFIT1, IFI44L, SIGLEC1, | 0.894 | 0.886 | 0.903 | 0.928 | 0.873 | 0.983 | 0.940 | 0.867 | 1 |
| ISG15, HERC6, FAM20A, RETN | | | | | | | | | |
| RSAD2, IFI27, IFI44L, SIGLEC1, | 0.894 | 0.886 | 0.903 | 0.935 | 0.885 | 0.985 | 0.952 | 0.893 | 1 |
| IL1R2, OLAH, FAM20A, MMP8 | | | | | | | | | |
| RSAD2, IFI27, SIGLEC1, ISG15, | 0.894 | 0.885 | 0.903 | 0.934 | 0.883 | 0.984 | 0.952 | 0.885 | 1 |
| HERC6, IL1R2, FAM20A, MMP8 | | | | | | | | | |
| IFI27, OAS1, IFI44L, SIGLEC1, | 0.894 | 0.885 | 0.903 | 0.933 | 0.880 | 0.985 | 0.949 | 0.886 | 1 |
| HERC6, OLAH, FAM20A, MMP8 | | | | | | | | | |
| IFI27, IFIT1, IFI44L, SIGLEC1, | 0.894 | 0.886 | 0.902 | 0.933 | 0.882 | 0.983 | 0.943 | 0.875 | 1 |
| ISG15, HERC6, IL1R2, FAM20A | | | | | | | | | |
| IFI27, OAS1, IFIT1, HERC6, IL1R2, | 0.894 | 0.885 | 0.903 | 0.932 | 0.881 | 0.984 | 0.930 | 0.857 | 1 |
| OLAH, FAM20A, MMP8 | | | | | | | | | |
| RSAD2, IFI27, IFI44L, HERC6, | 0.894 | 0.885 | 0.903 | 0.932 | 0.881 | 0.983 | 0.930 | 0.857 | 1 |
| IL1R2, OLAH, FAM20A, MMP8 | | | | | | | | | |
| IFI27, IFI44L, SIGLEC1, ISG15, | 0.894 | 0.885 | 0.903 | 0.934 | 0.883 | 0.986 | 0.951 | 0.882 | 1 |
| IL1R2, FAM20A, RETN, MMP8 | | | | | | | | | |
| IFI27, OAS1, IFIT1, SIGLEC1, | 0.894 | 0.885 | 0.903 | 0.935 | 0.885 | 0.985 | 0.943 | 0.876 | 1 |
| HERC6, IL1R2, FAM20A, RETN | | | | | | | | | |
| RSAD2, IFI27, SIGLEC1, IL1R2, | 0.894 | 0.885 | 0.903 | 0.934 | 0.881 | 0.988 | 0.951 | 0.890 | 1 |
| OLAH, FAM20A, RETN, MMP8 | | | | | | | | | |
| IFI27, OAS1, SIGLEC1, ISG15, | 0.894 | 0.885 | 0.903 | 0.932 | 0.881 | 0.983 | 0.950 | 0.882 | 1 |
| HERC6, IL1R2, FAM20A, MMP8 | | | | | | | | | |
| IFI27, OAS1, SIGLEC1, IL1R2, | 0.894 | 0.884 | 0.903 | 0.933 | 0.878 | 0.987 | 0.952 | 0.893 | 1 |
| OLAH, FAM20A, RETN, MMP8 | | | | | | | | | |
| RSAD2, IFI27, IFI44L, SIGLEC1, | 0.894 | 0.885 | 0.903 | 0.934 | 0.881 | 0.986 | 0.950 | 0.889 | 1 |
| HERC6, OLAH, FAM20A, MMP8 | | | | | | | | | |
| RSAD2, IFI27, IFIT1, SIGLEC1, | 0.894 | 0.885 | 0.903 | 0.929 | 0.872 | 0.986 | 0.950 | 0.893 | 1 |
| ISG15, OLAH, FAM20A, MMP8 | | | | | | | | | |
| IFI27, OAS1, SIGLEC1, ISG15, | 0.894 | 0.885 | 0.903 | 0.930 | 0.876 | 0.984 | 0.942 | 0.877 | 1 |
| HERC6, IL1R2, OLAH, FAM20A | | | | | | | | | |
| IFI27, IFI44L, SIGLEC1, HERC6, | 0.894 | 0.885 | 0.903 | 0.935 | 0.884 | 0.986 | 0.952 | 0.883 | 1 |
| IL1R2, FAM20A, RETN, MMP8 | | | | | | | | | |
| IFI27, OAS1, IFIT1, IFI44L, | 0.894 | 0.885 | 0.902 | 0.930 | 0.877 | 0.984 | 0.936 | 0.864 | 1 |
| SIGLEC1, HERC6, FAM20A, RETN | | | | | | | | | |
| RSAD2, IFI27, OAS1, IFIT1, | 0.894 | 0.885 | 0.903 | 0.928 | 0.871 | 0.985 | 0.954 | 0.899 | 1 |
| SIGLEC1, OLAH, FAM20A, MMP8 | | | | | | | | | |
| IFI27, IFIT1, IFI44L, SIGLEC1, | 0.894 | 0.885 | 0.902 | 0.931 | 0.878 | 0.985 | 0.948 | 0.880 | 1 |
| HERC6, IL1R2, FAM20A, RETN | | | | | | | | | |
| IFI27, IFIT1, SIGLEC1, ISG15, | 0.894 | 0.884 | 0.903 | 0.939 | 0.892 | 0.987 | 0.948 | 0.885 | 1 |
| SLC1A2, OLAH, FAM20A, MMP8 | | | | | | | | | |
| IFI27, OAS1, IFI44L, SIGLEC1, | 0.894 | 0.885 | 0.902 | 0.931 | 0.879 | 0.983 | 0.942 | 0.874 | 1 |
| HERC6, IL1R2, OLAH, FAM20A | | | | | | | | | |
| IFI27, OAS1, ISG15, HERC6, | 0.894 | 0.884 | 0.903 | 0.930 | 0.878 | 0.982 | 0.925 | 0.848 | 1 |
| IL1R2, OLAH, FAM20A, MMP8 | | | | | | | | | |
| IFI27, IFI44L, SIGLEC1, IL1R2, | 0.894 | 0.885 | 0.902 | 0.934 | 0.881 | 0.987 | 0.952 | 0.889 | 1 |
| OLAH, FAM20A, RETN, MMP8 | | | | | | | | | |
| RSAD2, IFI27, IFIT1, SIGLEC1, | 0.894 | 0.884 | 0.903 | 0.931 | 0.877 | 0.985 | 0.939 | 0.875 | 1 |
| ISG15, HERC6, OLAH, FAM20A | | | | | | | | | |
| IFI27, IFI44L, ISG15, HERC6, | 0.893 | 0.884 | 0.903 | 0.930 | 0.877 | 0.983 | 0.930 | 0.858 | 1 |
| IL1R2, OLAH, FAM20A, MMP8 | | | | | | | | | |
| RSAD2, IFI27, IFIT1, IFI44L, | 0.893 | 0.885 | 0.901 | 0.932 | 0.881 | 0.982 | 0.958 | 0.897 | 1 |
| SIGLEC1, IL1R2, FAM20A, MMP8 | | | | | | | | | |
| IFI27, IFIT1, IFI44L, HERC6, | 0.893 | 0.884 | 0.902 | 0.931 | 0.880 | 0.983 | 0.936 | 0.864 | 1 |
| IL1R2, OLAH, FAM20A, MMP8 | | | | | | | | | |
| IFI27, SIGLEC1, ISG15, SLC1A2, | 0.893 | 0.884 | 0.902 | 0.940 | 0.895 | 0.985 | 0.945 | 0.875 | 1 |
| IL1R2, OLAH, FAM20A, MMP8 | | | | | | | | | |
| RSAD2, IFI27, IFIT1, SIGLEC1, | 0.893 | 0.884 | 0.902 | 0.931 | 0.877 | 0.985 | 0.939 | 0.875 | 1 |
| HERC6, OLAH, FAM20A, RETN | | | | | | | | | |
| RSAD2, IFI27, IFIT1, HERC6, | 0.893 | 0.884 | 0.902 | 0.931 | 0.880 | 0.983 | 0.934 | 0.863 | 1 |
| IL1R2, OLAH, FAM20A, MMP8 | | | | | | | | | |
| IFI27, OAS1, IFIT1, SIGLEC1, | 0.893 | 0.885 | 0.902 | 0.934 | 0.884 | 0.983 | 0.947 | 0.881 | 1 |
| ISG15, HERC6, IL1R2, FAM20A | | | | | | | | | |
| IFI27, OAS1, IFIT1, SIGLEC1, | 0.893 | 0.884 | 0.903 | 0.928 | 0.870 | 0.986 | 0.957 | 0.902 | 1 |
| OLAH, FAM20A, RETN, MMP8 | | | | | | | | | |
| IFI27, IFIT1, SIGLEC1, ISG15, | 0.893 | 0.884 | 0.902 | 0.934 | 0.883 | 0.986 | 0.955 | 0.891 | 1 |
| IL1R2, FAM20A, RETN, MMP8 | | | | | | | | | |
| IFI27, OAS1, IFI44L, HERC6, | 0.893 | 0.883 | 0.903 | 0.931 | 0.879 | 0.983 | 0.927 | 0.851 | 1 |
| IL1R2, OLAH, FAM20A, MMP8 | | | | | | | | | |
| IFI27, OAS1, IFIT1, SIGLEC1, | 0.893 | 0.884 | 0.902 | 0.931 | 0.878 | 0.984 | 0.936 | 0.864 | 1 |
| ISG15, HERC6, FAM20A, RETN | | | | | | | | | |
| IFI27, IFIT1, SIGLEC1, ISG15, | 0.893 | 0.883 | 0.902 | 0.930 | 0.873 | 0.987 | 0.955 | 0.901 | 1 |
| OLAH, FAM20A, RETN, MMP8 | | | | | | | | | |
| IFI27, OAS1, IFIT1, SIGLEC1, | 0.893 | 0.884 | 0.902 | 0.933 | 0.882 | 0.983 | 0.954 | 0.893 | 1 |
| ISG15, IL1R2, FAM20A, MMP8 | | | | | | | | | |
| IFI27, IFIT1, IFI44L, SIGLEC1, | 0.893 | 0.884 | 0.902 | 0.926 | 0.869 | 0.983 | 0.947 | 0.875 | 1 |
| HERC6, FAM20A, RETN, MMP8 | | | | | | | | | |
| RSAD2, IFI27, SIGLEC1, ISG15, | 0.893 | 0.884 | 0.902 | 0.930 | 0.876 | 0.985 | 0.942 | 0.879 | 1 |
| HERC6, IL1R2, OLAH, FAM20A | | | | | | | | | |
| IFI27, OAS1, IFIT1, IFI44L, | 0.893 | 0.884 | 0.901 | 0.927 | 0.870 | 0.983 | 0.948 | 0.885 | 1 |
| SIGLEC1, IL1R2, OLAH, FAM20A | | | | | | | | | |
| RSAD2, IFI27, OAS1, IFIT1, | 0.893 | 0.884 | 0.901 | 0.932 | 0.880 | 0.983 | 0.955 | 0.894 | 1 |
| SIGLEC1, IL1R2, FAM20A, MMP8 | | | | | | | | | |
| IFI27, IFIT1, IFI44L, SIGLEC1, | 0.893 | 0.884 | 0.901 | 0.928 | 0.872 | 0.984 | 0.946 | 0.883 | 1 |
| ISG15, IL1R2, OLAH, FAM20A | | | | | | | | | |
| RSAD2, IFI27, OAS1, IFIT1, | 0.893 | 0.884 | 0.901 | 0.934 | 0.885 | 0.983 | 0.946 | 0.879 | 1 |
| SIGLEC1, HERC6, IL1R2, FAM20A | | | | | | | | | |
| IFI27, IFIT1, SIGLEC1, HERC6, | 0.893 | 0.884 | 0.902 | 0.932 | 0.879 | 0.986 | 0.942 | 0.878 | 1 |
| IL1R2, OLAH, FAM20A, RETN | | | | | | | | | |
| RSAD2, IFI27, OAS1, IFI44L, | 0.893 | 0.884 | 0.902 | 0.932 | 0.881 | 0.983 | 0.953 | 0.889 | 1 |
| SIGLEC1, IL1R2, FAM20A, MMP8 | | | | | | | | | |
| RSAD2, IFI27, IFIT1, IFI44L, | 0.893 | 0.884 | 0.901 | 0.933 | 0.882 | 0.983 | 0.946 | 0.878 | 1 |
| SIGLEC1, HERC6, IL1R2, FAM20A | | | | | | | | | |
| RSAD2, IFI27, OAS1, SIGLEC1, | 0.893 | 0.883 | 0.902 | 0.930 | 0.873 | 0.987 | 0.946 | 0.884 | 1 |
| ISG15, OLAH, FAM20A, MMP8 | | | | | | | | | |
| RSAD2, IFI27, ISG15, HERC6, | 0.893 | 0.883 | 0.902 | 0.930 | 0.879 | 0.982 | 0.925 | 0.849 | 1 |
| IL1R2, OLAH, FAM20A, MMP8 | | | | | | | | | |
| IFI27, OAS1, IFI44L, SIGLEC1, | 0.892 | 0.883 | 0.902 | 0.927 | 0.870 | 0.985 | 0.949 | 0.891 | 1 |
| ISG15, OLAH, FAM20A, MMP8 | | | | | | | | | |
| IFI27, OAS1, IFI44L, SIGLEC1, | 0.892 | 0.883 | 0.902 | 0.930 | 0.876 | 0.985 | 0.940 | 0.874 | 1 |
| ISG15, HERC6, OLAH, FAM20A | | | | | | | | | |
| RSAD2, IFI27, IFIT1, SIGLEC1, | 0.892 | 0.884 | 0.901 | 0.933 | 0.883 | 0.983 | 0.954 | 0.890 | 1 |
| ISG15, IL1R2, FAM20A, MMP8 | | | | | | | | | |
| IFI27, IFI44L, SIGLEC1, ISG15, | 0.892 | 0.883 | 0.901 | 0.936 | 0.887 | 0.984 | 0.947 | 0.882 | 1 |
| SLC1A2, OLAH, FAM20A, MMP8 | | | | | | | | | |
| IFI27, OAS1, SIGLEC1, ISG15, | 0.892 | 0.883 | 0.902 | 0.938 | 0.889 | 0.987 | 0.941 | 0.875 | 1 |
| SLC1A2, OLAH, FAM20A, MMP8 | | | | | | | | | |
| RSAD2, IFI27, IFI44L, SIGLEC1, | 0.892 | 0.884 | 0.901 | 0.933 | 0.880 | 0.985 | 0.947 | 0.883 | 1 |
| HERC6, IL1R2, OLAH, FAM20A | | | | | | | | | |
| RSAD2, IFI27, OAS1, SIGLEC1, | 0.892 | 0.883 | 0.902 | 0.931 | 0.877 | 0.986 | 0.937 | 0.869 | 1 |
| ISG15, HERC6, OLAH, FAM20A | | | | | | | | | |
| IFI27, OAS1, IFI44L, SIGLEC1, | 0.892 | 0.883 | 0.901 | 0.934 | 0.884 | 0.984 | 0.950 | 0.882 | 1 |
| ISG15, IL1R2, FAM20A, MMP8 | | | | | | | | | |
| RSAD2, IFI27, OAS1, IFI44L, | 0.892 | 0.883 | 0.901 | 0.931 | 0.877 | 0.984 | 0.940 | 0.875 | 1 |
| SIGLEC1, HERC6, OLAH, FAM20A | | | | | | | | | |
| IFI27, OAS1, SIGLEC1, ISG15, | 0.892 | 0.883 | 0.902 | 0.930 | 0.876 | 0.985 | 0.940 | 0.874 | 1 |
| HERC6, OLAH, FAM20A, RETN | | | | | | | | | |
| IFI27, OAS1, IFIT1, IFI44L, | 0.892 | 0.884 | 0.901 | 0.926 | 0.868 | 0.984 | 0.943 | 0.884 | 1 |
| SIGLEC1, ISG15, OLAH, FAM20A | | | | | | | | | |
| IFI27, IFI44L, SIGLEC1, ISG15, | 0.892 | 0.883 | 0.902 | 0.929 | 0.872 | 0.986 | 0.951 | 0.891 | 1 |
| OLAH, FAM20A, RETN, MMP8 | | | | | | | | | |
| IFI27, IFIT1, IFI44L, SIGLEC1, | 0.892 | 0.883 | 0.901 | 0.934 | 0.883 | 0.986 | 0.952 | 0.890 | 1 |
| SLC1A2, OLAH, FAM20A, MMP8 | | | | | | | | | |
| IFI27, IFIT1, SIGLEC1, SLC1A2, | 0.892 | 0.883 | 0.901 | 0.939 | 0.894 | 0.985 | 0.954 | 0.893 | 1 |
| IL1R2, OLAH, FAM20A, MMP8 | | | | | | | | | |
| RSAD2, IFI27, IFIT1, SIGLEC1, | 0.892 | 0.883 | 0.901 | 0.935 | 0.883 | 0.987 | 0.952 | 0.888 | 1 |
| IL1R2, FAM20A, RETN, MMP8 | | | | | | | | | |
| RSAD2, IFI27, OAS1, SIGLEC1, | 0.892 | 0.882 | 0.902 | 0.934 | 0.882 | 0.986 | 0.953 | 0.889 | 1 |
| IL1R2, FAM20A, RETN, MMP8 | | | | | | | | | |
| IFI27, IFIT1, IFI44L, SIGLEC1, | 0.892 | 0.882 | 0.901 | 0.930 | 0.874 | 0.987 | 0.959 | 0.906 | 1 |
| OLAH, FAM20A, RETN, MMP8 | | | | | | | | | |
| IFI27, OAS1, SIGLEC1, ISG15, | 0.892 | 0.883 | 0.901 | 0.933 | 0.879 | 0.986 | 0.951 | 0.885 | 1 |
| IL1R2, FAM20A, RETN, MMP8 | | | | | | | | | |
| RSAD2, IFI27, IFIT1, SIGLEC1, | 0.892 | 0.883 | 0.901 | 0.931 | 0.878 | 0.985 | 0.942 | 0.870 | 1 |
| HERC6, FAM20A, RETN, MMP8 | | | | | | | | | |
| IFI27, OAS1, IFI44L, SIGLEC1, | 0.892 | 0.883 | 0.901 | 0.933 | 0.881 | 0.985 | 0.952 | 0.888 | 1 |
| IL1R2, FAM20A, RETN, MMP8 | | | | | | | | | |
| IFI27, IFIT1, SIGLEC1, ISG15, | 0.892 | 0.883 | 0.901 | 0.927 | 0.870 | 0.984 | 0.949 | 0.878 | 1 |
| HERC6, FAM20A, RETN, MMP8 | | | | | | | | | |
| IFI27, IFIT1, SIGLEC1, ISG15, | 0.892 | 0.883 | 0.900 | 0.929 | 0.872 | 0.986 | 0.946 | 0.884 | 1 |
| IL1R2, OLAH, FAM20A, RETN | | | | | | | | | |
| RSAD2, IFI27, IFIT1, IFI44L, | 0.892 | 0.883 | 0.901 | 0.928 | 0.871 | 0.985 | 0.954 | 0.899 | 1 |
| SIGLEC1, OLAH, FAM20A, MMP8 | | | | | | | | | |
| IFI27, OAS1, HERC6, IL1R2, | 0.892 | 0.883 | 0.901 | 0.935 | 0.883 | 0.986 | 0.929 | 0.855 | 1 |
| OLAH, FAM20A, RETN, MMP8 | | | | | | | | | |
| RSAD2, IFI27, HERC6, IL1R2, | 0.892 | 0.882 | 0.901 | 0.934 | 0.882 | 0.986 | 0.932 | 0.858 | 1 |
| OLAH, FAM20A, RETN, MMP8 | | | | | | | | | |
| IFI27, OAS1, IFIT1, IFI44L, | 0.892 | 0.882 | 0.901 | 0.929 | 0.874 | 0.983 | 0.934 | 0.865 | 1 |
| HERC6, OLAH, FAM20A, MMP8 | | | | | | | | | |
| IFI27, IFIT1, ISG15, HERC6, IL1R2, | 0.892 | 0.883 | 0.901 | 0.930 | 0.877 | 0.982 | 0.929 | 0.857 | 1 |
| OLAH, FAM20A, MMP8 | | | | | | | | | |
| RSAD2, IFI27, SIGLEC1, SLC1A2, | 0.892 | 0.883 | 0.901 | 0.937 | 0.890 | 0.984 | 0.950 | 0.888 | 1 |
| IL1R2, OLAH, FAM20A, MMP8 | | | | | | | | | |
| IFI27, OAS1, SIGLEC1, HERC6, | 0.892 | 0.882 | 0.901 | 0.934 | 0.884 | 0.984 | 0.941 | 0.877 | 1 |
| SLC1A2, OLAH, FAM20A, MMP8 | | | | | | | | | |
| RSAD2, IFI27, SIGLEC1, ISG15, | 0.892 | 0.882 | 0.901 | 0.935 | 0.883 | 0.987 | 0.949 | 0.880 | 1 |
| IL1R2, FAM20A, RETN, MMP8 | | | | | | | | | |
| IFI27, IFIT1, IFI44L, SIGLEC1, | 0.892 | 0.883 | 0.900 | 0.927 | 0.870 | 0.985 | 0.942 | 0.880 | 1 |
| ISG15, OLAH, FAM20A, RETN | | | | | | | | | |
| IFI27, OAS1, IFIT1, SIGLEC1, | 0.892 | 0.883 | 0.900 | 0.926 | 0.869 | 0.983 | 0.949 | 0.887 | 1 |
| IL1R2, OLAH, FAM20A, RETN | | | | | | | | | |
| IFI27, OAS1, SIGLEC1, ISG15, | 0.892 | 0.881 | 0.902 | 0.928 | 0.870 | 0.986 | 0.951 | 0.893 | 1 |
| OLAH, FAM20A, RETN, MMP8 | | | | | | | | | |
| IFI27, SIGLEC1, ISG15, HERC6, | 0.892 | 0.881 | 0.902 | 0.928 | 0.870 | 0.986 | 0.957 | 0.899 | 1 |
| OLAH, FAM20A, RETN, MMP8 | | | | | | | | | |
| RSAD2, IFI27, OAS1, IFI44L, | 0.892 | 0.882 | 0.901 | 0.930 | 0.876 | 0.984 | 0.934 | 0.866 | 1 |
| HERC6, OLAH, FAM20A, MMP8 | | | | | | | | | |
| IFI27, OAS1, IFI44L, SIGLEC1, | 0.891 | 0.883 | 0.900 | 0.931 | 0.881 | 0.982 | 0.946 | 0.879 | 1 |
| ISG15, HERC6, IL1R2, FAM20A | | | | | | | | | |
| IFI27, OAS1, SIGLEC1, HERC6, | 0.891 | 0.882 | 0.901 | 0.930 | 0.876 | 0.983 | 0.939 | 0.871 | 1 |
| IL1R2, OLAH, FAM20A, RETN | | | | | | | | | |
| IFI27, IFIT1, SIGLEC1, ISG15, | 0.891 | 0.882 | 0.900 | 0.933 | 0.880 | 0.986 | 0.950 | 0.883 | 1 |
| HERC6, IL1R2, FAM20A, RETN | | | | | | | | | |
| IFI27, IFIT1, IFI44L, SIGLEC1, | 0.891 | 0.883 | 0.900 | 0.927 | 0.870 | 0.983 | 0.947 | 0.884 | 1 |
| IL1R2, OLAH, FAM20A, RETN | | | | | | | | | |
| IFI27, ISG15, HERC6, IL1R2, | 0.891 | 0.882 | 0.901 | 0.932 | 0.879 | 0.985 | 0.932 | 0.859 | 1 |
| OLAH, FAM20A, RETN, MMP8 | | | | | | | | | |
| IFI27, OAS1, IFIT1, SIGLEC1, | 0.891 | 0.883 | 0.900 | 0.928 | 0.871 | 0.985 | 0.943 | 0.881 | 1 |
| ISG15, IL1R2, OLAH, FAM20A | | | | | | | | | |
| RSAD2, IFI27, IFIT1, IFI44L, | 0.891 | 0.882 | 0.901 | 0.930 | 0.875 | 0.984 | 0.934 | 0.866 | 1 |
| HERC6, OLAH, FAM20A, MMP8 | | | | | | | | | |
| IFI27, SIGLEC1, ISG15, HERC6, | 0.891 | 0.882 | 0.901 | 0.933 | 0.880 | 0.986 | 0.953 | 0.885 | 1 |
| IL1R2, FAM20A, RETN, MMP8 | | | | | | | | | |
| IFI27, IFI44L, SIGLEC1, ISG15, | 0.891 | 0.883 | 0.900 | 0.928 | 0.872 | 0.983 | 0.946 | 0.882 | 1 |
| IL1R2, OLAH, FAM20A, RETN | | | | | | | | | |
| IFI27, OAS1, IFI44L, SIGLEC1, | 0.891 | 0.883 | 0.900 | 0.934 | 0.884 | 0.984 | 0.943 | 0.876 | 1 |
| HERC6, IL1R2, FAM20A, RETN | | | | | | | | | |
| IFI27, OAS1, IFI44L, SIGLEC1, | 0.891 | 0.882 | 0.900 | 0.931 | 0.878 | 0.984 | 0.939 | 0.871 | 1 |
| HERC6, OLAH, FAM20A, RETN | | | | | | | | | |
| RSAD2, IFI27, IFIT1, SIGLEC1, | 0.891 | 0.883 | 0.900 | 0.934 | 0.884 | 0.985 | 0.948 | 0.882 | 1 |
| ISG15, HERC6, IL1R2, FAM20A | | | | | | | | | |
| IFI27, SIGLEC1, ISG15, HERC6, | 0.891 | 0.882 | 0.901 | 0.935 | 0.885 | 0.986 | 0.945 | 0.878 | 1 |
| SLC1A2, OLAH, FAM20A, MMP8 | | | | | | | | | |
| RSAD2, IFI27, OAS1, IFIT1, | 0.891 | 0.882 | 0.900 | 0.930 | 0.875 | 0.984 | 0.930 | 0.859 | 1 |
| IFI44L, HERC6, OLAH, FAM20A | | | | | | | | | |
| RSAD2, IFI27, OAS1, SIGLEC1, | 0.891 | 0.882 | 0.901 | 0.930 | 0.876 | 0.985 | 0.936 | 0.867 | 1 |
| HERC6, OLAH, FAM20A, RETN | | | | | | | | | |
| RSAD2, IFI27, IFI44L, SIGLEC1, | 0.891 | 0.882 | 0.900 | 0.933 | 0.883 | 0.984 | 0.950 | 0.882 | 1 |
| ISG15, IL1R2, FAM20A, MMP8 | | | | | | | | | |
| IFI27, OAS1, IFIT1, IFI44L, | 0.891 | 0.882 | 0.900 | 0.924 | 0.865 | 0.983 | 0.951 | 0.891 | 1 |
| SIGLEC1, OLAH, FAM20A, RETN | | | | | | | | | |
| RSAD2, IFI27, OAS1, IFIT1, | 0.891 | 0.882 | 0.900 | 0.930 | 0.877 | 0.984 | 0.934 | 0.861 | 1 |
| SIGLEC1, HERC6, FAM20A, RETN | | | | | | | | | |
| RSAD2, IFI27, OAS1, SIGLEC1, | 0.891 | 0.882 | 0.901 | 0.934 | 0.883 | 0.985 | 0.951 | 0.883 | 1 |
| ISG15, IL1R2, FAM20A, MMP8 | | | | | | | | | |
| RSAD2, IFI27, OAS1, IFIT1, | 0.891 | 0.882 | 0.901 | 0.930 | 0.877 | 0.983 | 0.934 | 0.865 | 1 |
| HERC6, OLAH, FAM20A, MMP8 | | | | | | | | | |
| IFI27, IFI44L, SIGLEC1, SLC1A2, | 0.891 | 0.882 | 0.900 | 0.935 | 0.888 | 0.983 | 0.950 | 0.884 | 1 |
| IL1R2, OLAH, FAM20A, MMP8 | | | | | | | | | |
| RSAD2, IFI27, OAS1, IFI44L, | 0.891 | 0.883 | 0.900 | 0.933 | 0.884 | 0.983 | 0.946 | 0.880 | 1 |
| SIGLEC1, HERC6, IL1R2, FAM20A | | | | | | | | | |
| IFI27, IFI44L, SIGLEC1, HERC6, | 0.891 | 0.881 | 0.901 | 0.928 | 0.872 | 0.984 | 0.950 | 0.885 | 1 |
| OLAH, FAM20A, RETN, MMP8 | | | | | | | | | |
| RSAD2, IFI27, SIGLEC1, ISG15, | 0.891 | 0.882 | 0.901 | 0.938 | 0.890 | 0.986 | 0.941 | 0.875 | 1 |
| SLC1A2, OLAH, FAM20A, MMP8 | | | | | | | | | |
| IFI27, OAS1, IFIT1, IFI44L, | 0.891 | 0.882 | 0.900 | 0.930 | 0.878 | 0.983 | 0.925 | 0.848 | 1 |
| HERC6, IL1R2, OLAH, FAM20A | | | | | | | | | |
| RSAD2, IFI27, IFIT1, SIGLEC1, | 0.891 | 0.882 | 0.900 | 0.933 | 0.880 | 0.985 | 0.948 | 0.880 | 1 |
| HERC6, IL1R2, FAM20A, RETN | | | | | | | | | |
| IFI27, OAS1, ISG15, HERC6, | 0.891 | 0.882 | 0.901 | 0.931 | 0.876 | 0.986 | 0.930 | 0.856 | 1 |
| OLAH, FAM20A, RETN, MMP8 | | | | | | | | | |
| RSAD2, IFI27, IFI44L, SIGLEC1, | 0.891 | 0.882 | 0.900 | 0.928 | 0.873 | 0.984 | 0.941 | 0.877 | 1 |
| ISG15, HERC6, OLAH, FAM20A | | | | | | | | | |
| RSAD2, IFI27, OAS1, IFI44L, | 0.891 | 0.881 | 0.901 | 0.927 | 0.869 | 0.985 | 0.954 | 0.900 | 1 |
| SIGLEC1, OLAH, FAM20A, MMP8 | | | | | | | | | |
| IFI27, OAS1, IFIT1, SIGLEC1, | 0.891 | 0.882 | 0.900 | 0.937 | 0.887 | 0.986 | 0.946 | 0.883 | 1 |
| SLC1A2, OLAH, FAM20A, MMP8 | | | | | | | | | |
| IFI27, OAS1, SIGLEC1, SLC1A2, | 0.891 | 0.882 | 0.900 | 0.937 | 0.889 | 0.984 | 0.945 | 0.879 | 1 |
| IL1R2, OLAH, FAM20A, MMP8 | | | | | | | | | |
| RSAD2, IFI27, OAS1, SIGLEC1, | 0.891 | 0.881 | 0.901 | 0.928 | 0.870 | 0.986 | 0.958 | 0.905 | 1 |
| OLAH, FAM20A, RETN, MMP8 | | | | | | | | | |
| RSAD2, IFI27, SIGLEC1, ISG15, | 0.891 | 0.881 | 0.901 | 0.930 | 0.872 | 0.987 | 0.950 | 0.893 | 1 |
| OLAH, FAM20A, RETN, MMP8 | | | | | | | | | |
| RSAD2, IFI27, OAS1, HERC6, | 0.891 | 0.881 | 0.900 | 0.931 | 0.876 | 0.985 | 0.929 | 0.855 | 1 |
| OLAH, FAM20A, RETN, MMP8 | | | | | | | | | |
| RSAD2, IFI27, IFI44L, SIGLEC1, | 0.891 | 0.881 | 0.900 | 0.934 | 0.882 | 0.986 | 0.951 | 0.883 | 1 |
| IL1R2, FAM20A, RETN, MMP8 | | | | | | | | | |
| IFI27, OAS1, IFIT1, SIGLEC1, | 0.891 | 0.881 | 0.901 | 0.937 | 0.890 | 0.984 | 0.938 | 0.871 | 1 |
| HERC6, SLC1A2, OLAH, FAM20A | | | | | | | | | |
| IFI27, IFI44L, SIGLEC1, ISG15, | 0.891 | 0.881 | 0.900 | 0.926 | 0.870 | 0.983 | 0.943 | 0.878 | 1 |
| HERC6, OLAH, FAM20A, RETN | | | | | | | | | |
| RSAD2, IFI27, IFIT1, IFI44L, | 0.891 | 0.882 | 0.900 | 0.929 | 0.875 | 0.983 | 0.935 | 0.861 | 1 |
| SIGLEC1, HERC6, FAM20A, RETN | | | | | | | | | |
| IFI27, OAS1, IFIT1, HERC6, OLAH, | 0.891 | 0.881 | 0.901 | 0.932 | 0.878 | 0.987 | 0.932 | 0.859 | 1 |
| FAM20A, RETN, MMP8 | | | | | | | | | |
| RSAD2, IFI27, SIGLEC1, HERC6, | 0.891 | 0.882 | 0.900 | 0.931 | 0.877 | 0.985 | 0.945 | 0.881 | 1 |
| IL1R2, OLAH, FAM20A, RETN | | | | | | | | | |
| RSAD2, IFI27, OAS1, IFIT1, | 0.891 | 0.882 | 0.900 | 0.923 | 0.864 | 0.983 | 0.950 | 0.891 | 1 |
| IFI44L, SIGLEC1, OLAH, FAM20A | | | | | | | | | |
| IFI27, OAS1, SIGLEC1, ISG15, | 0.891 | 0.881 | 0.901 | 0.929 | 0.872 | 0.986 | 0.943 | 0.872 | 1 |
| HERC6, FAM20A, RETN, MMP8 | | | | | | | | | |
| IFI27, OAS1, IFI44L, ISG15, | 0.891 | 0.881 | 0.900 | 0.929 | 0.876 | 0.982 | 0.927 | 0.853 | 1 |
| HERC6, IL1R2, OLAH, FAM20A | | | | | | | | | |
| RSAD2, IFI27, OAS1, SIGLEC1, | 0.891 | 0.881 | 0.900 | 0.933 | 0.878 | 0.987 | 0.942 | 0.872 | 1 |
| HERC6, FAM20A, RETN, MMP8 | | | | | | | | | |
| IFI27, IFIT1, SIGLEC1, HERC6, | 0.891 | 0.881 | 0.900 | 0.935 | 0.886 | 0.985 | 0.953 | 0.893 | 1 |
| SLC1A2, OLAH, FAM20A, MMP8 | | | | | | | | | |
| IFI27, SIGLEC1, HERC6, IL1R2, | 0.891 | 0.881 | 0.901 | 0.931 | 0.876 | 0.986 | 0.957 | 0.898 | 1 |
| OLAH, FAM20A, RETN, MMP8 | | | | | | | | | |
| IFI27, OAS1, IFI44L, SIGLEC1, | 0.891 | 0.882 | 0.899 | 0.927 | 0.871 | 0.983 | 0.947 | 0.885 | 1 |
| ISG15, IL1R2, OLAH, FAM20A | | | | | | | | | |
| RSAD2, IFI27, OAS1, IFI44L, | 0.891 | 0.881 | 0.900 | 0.931 | 0.878 | 0.983 | 0.929 | 0.856 | 1 |
| HERC6, IL1R2, OLAH, FAM20A | | | | | | | | | |
| RSAD2, IFI27, IFIT1, SIGLEC1, | 0.891 | 0.881 | 0.900 | 0.929 | 0.872 | 0.987 | 0.955 | 0.902 | 1 |
| OLAH, FAM20A, RETN, MMP8 | | | | | | | | | |
| IFI27, IFI44L, ISG15, SLC1A2, | 0.891 | 0.882 | 0.899 | 0.935 | 0.888 | 0.982 | 0.923 | 0.844 | 1 |
| IL1R2, OLAH, FAM20A, MMP8 | | | | | | | | | |
| RSAD2, IFI27, OAS1, ISG15, | 0.890 | 0.881 | 0.900 | 0.927 | 0.873 | 0.982 | 0.932 | 0.862 | 1 |
| HERC6, OLAH, FAM20A, MMP8 | | | | | | | | | |
| IFI27, IFIT1, IFI44L, ISG15, | 0.890 | 0.881 | 0.900 | 0.926 | 0.870 | 0.982 | 0.936 | 0.870 | 1 |
| HERC6, OLAH, FAM20A, MMP8 | | | | | | | | | |
| RSAD2, IFI27, OAS1, IFIT1, IL1R2, | 0.890 | 0.881 | 0.899 | 0.929 | 0.874 | 0.983 | 0.936 | 0.868 | 1 |
| OLAH, FAM20A, MMP8 | | | | | | | | | |
| RSAD2, IFI27, IFI44L, SIGLEC1, | 0.890 | 0.881 | 0.900 | 0.929 | 0.872 | 0.985 | 0.950 | 0.893 | 1 |
| ISG15, OLAH, FAM20A, MMP8 | | | | | | | | | |
| RSAD2, IFI27, IFIT1, IFI44L, | 0.890 | 0.882 | 0.899 | 0.927 | 0.871 | 0.984 | 0.947 | 0.884 | 1 |
| SIGLEC1, IL1R2, OLAH, FAM20A | | | | | | | | | |
| RSAD2, IFI27, SIGLEC1, ISG15, | 0.890 | 0.881 | 0.900 | 0.931 | 0.877 | 0.985 | 0.941 | 0.878 | 1 |
| HERC6, OLAH, FAM20A, RETN | | | | | | | | | |
| IFI27, OAS1, IFIT1, SIGLEC1, | 0.890 | 0.881 | 0.899 | 0.926 | 0.867 | 0.984 | 0.943 | 0.882 | 1 |
| ISG15, OLAH, FAM20A, RETN | | | | | | | | | |
| IFI27, IFIT1, IFI44L, SIGLEC1, | 0.890 | 0.881 | 0.899 | 0.935 | 0.885 | 0.985 | 0.939 | 0.873 | 1 |
| ISG15, SLC1A2, OLAH, FAM20A | | | | | | | | | |
| RSAD2, IFI27, OAS1, SIGLEC1, | 0.890 | 0.881 | 0.900 | 0.931 | 0.880 | 0.983 | 0.945 | 0.877 | 1 |
| HERC6, IL1R2, FAM20A, RETN | | | | | | | | | |
| RSAD2, IFI27, IFIT1, SIGLEC1, | 0.890 | 0.881 | 0.900 | 0.935 | 0.885 | 0.986 | 0.948 | 0.885 | 1 |
| SLC1A2, OLAH, FAM20A, MMP8 | | | | | | | | | |
| RSAD2, IFI27, OAS1, IFIT1, | 0.890 | 0.882 | 0.899 | 0.926 | 0.869 | 0.983 | 0.949 | 0.886 | 1 |
| SIGLEC1, IL1R2, OLAH, FAM20A | | | | | | | | | |
| IFI27, OAS1, SIGLEC1, HERC6, | 0.890 | 0.881 | 0.900 | 0.936 | 0.891 | 0.981 | 0.948 | 0.879 | 1 |
| SLC1A2, IL1R2, FAM20A, MMP8 | | | | | | | | | |
| IFI27, OAS1, IFI44L, SIGLEC1, | 0.890 | 0.881 | 0.900 | 0.931 | 0.876 | 0.985 | 0.943 | 0.871 | 1 |
| HERC6, FAM20A, RETN, MMP8 | | | | | | | | | |
| RSAD2, IFI27, SIGLEC1, HERC6, | 0.890 | 0.881 | 0.900 | 0.936 | 0.887 | 0.985 | 0.943 | 0.878 | 1 |
| SLC1A2, OLAH, FAM20A, MMP8 | | | | | | | | | |
| RSAD2, IFI27, OAS1, IFIT1, | 0.890 | 0.881 | 0.899 | 0.926 | 0.867 | 0.984 | 0.945 | 0.884 | 1 |
| SIGLEC1, ISG15, OLAH, FAM20A | | | | | | | | | |
| RSAD2, IFI27, OAS1, SIGLEC1, | 0.890 | 0.881 | 0.899 | 0.931 | 0.878 | 0.983 | 0.948 | 0.882 | 1 |
| ISG15, HERC6, IL1R2, FAM20A | | | | | | | | | |
| IFI27, OAS1, IFIT1, IFI44L, | 0.890 | 0.881 | 0.899 | 0.931 | 0.879 | 0.984 | 0.928 | 0.855 | 1 |
| HERC6, OLAH, FAM20A, RETN | | | | | | | | | |
| RSAD2, IFI27, IFI44L, SIGLEC1, | 0.890 | 0.881 | 0.899 | 0.930 | 0.878 | 0.982 | 0.942 | 0.873 | 1 |
| ISG15, HERC6, IL1R2, FAM20A | | | | | | | | | |
| IFI27, IFIT1, SIGLEC1, ISG15, | 0.890 | 0.880 | 0.900 | 0.938 | 0.892 | 0.984 | 0.936 | 0.868 | 1 |
| HERC6, SLC1A2, OLAH, FAM20A | | | | | | | | | |
| RSAD2, IFI27, OAS1, IFIT1, | 0.890 | 0.881 | 0.899 | 0.929 | 0.877 | 0.981 | 0.928 | 0.854 | 1 |
| HERC6, IL1R2, OLAH, FAM20A | | | | | | | | | |
| RSAD2, IFI27, IFIT1, SIGLEC1, | 0.890 | 0.881 | 0.899 | 0.929 | 0.875 | 0.983 | 0.935 | 0.861 | 1 |
| ISG15, HERC6, FAM20A, RETN | | | | | | | | | |
| IFI27, IFIT1, HERC6, IL1R2, | 0.890 | 0.881 | 0.899 | 0.931 | 0.878 | 0.985 | 0.934 | 0.861 | 1 |
| OLAH, FAM20A, RETN, MMP8 | | | | | | | | | |
| IFI27, OAS1, IFIT1, ISG15, HERC6, | 0.890 | 0.881 | 0.899 | 0.929 | 0.876 | 0.981 | 0.926 | 0.851 | 1 |
| IL1R2, OLAH, FAM20A | | | | | | | | | |
| RSAD2, IFI27, OAS1, ISG15, | 0.890 | 0.881 | 0.899 | 0.929 | 0.876 | 0.981 | 0.926 | 0.852 | 1 |
| HERC6, IL1R2, OLAH, FAM20A | | | | | | | | | |
| IFI27, IFI44L, SIGLEC1, HERC6, | 0.890 | 0.881 | 0.899 | 0.927 | 0.872 | 0.982 | 0.947 | 0.881 | 1 |
| IL1R2, OLAH, FAM20A, RETN | | | | | | | | | |
| IFI27, IFIT1, IFI44L, ISG15, | 0.890 | 0.881 | 0.899 | 0.929 | 0.875 | 0.983 | 0.928 | 0.855 | 1 |
| HERC6, IL1R2, OLAH, FAM20A | | | | | | | | | |
| RSAD2, IFI27, IFIT1, HERC6, | 0.890 | 0.880 | 0.899 | 0.929 | 0.874 | 0.985 | 0.932 | 0.859 | 1 |
| OLAH, FAM20A, RETN, MMP8 | | | | | | | | | |
| IFI27, OAS1, IFI44L, SIGLEC1, | 0.890 | 0.881 | 0.899 | 0.928 | 0.873 | 0.982 | 0.938 | 0.864 | 1 |
| ISG15, HERC6, FAM20A, RETN | | | | | | | | | |
| IFI27, OAS1, IFI44L, ISG15, | 0.890 | 0.880 | 0.900 | 0.928 | 0.873 | 0.983 | 0.930 | 0.860 | 1 |
| HERC6, OLAH, FAM20A, MMP8 | | | | | | | | | |
| IFI27, IFIT1, IFI44L, ISG15, IL1R2, | 0.890 | 0.881 | 0.899 | 0.928 | 0.874 | 0.982 | 0.934 | 0.864 | 1 |
| OLAH, FAM20A, MMP8 | | | | | | | | | |
| RSAD2, IFI27, ISG15, HERC6, | 0.890 | 0.880 | 0.899 | 0.931 | 0.877 | 0.985 | 0.932 | 0.859 | 1 |
| OLAH, FAM20A, RETN, MMP8 | | | | | | | | | |
| RSAD2, IFI27, IFIT1, IFI44L, | 0.890 | 0.881 | 0.899 | 0.929 | 0.875 | 0.983 | 0.928 | 0.855 | 1 |
| HERC6, IL1R2, OLAH, FAM20A | | | | | | | | | |
| IFI27, OAS1, IFIT1, IFI44L, ISG15, | 0.890 | 0.880 | 0.900 | 0.928 | 0.873 | 0.983 | 0.927 | 0.854 | 1 |
| HERC6, OLAH, FAM20A | | | | | | | | | |
| RSAD2, IFI27, IFIT1, SIGLEC1, | 0.890 | 0.881 | 0.898 | 0.929 | 0.873 | 0.985 | 0.945 | 0.881 | 1 |
| ISG15, IL1R2, OLAH, FAM20A | | | | | | | | | |
| IFI27, IFI44L, SIGLEC1, ISG15, | 0.890 | 0.881 | 0.898 | 0.938 | 0.895 | 0.981 | 0.952 | 0.883 | 1 |
| SLC1A2, IL1R2, FAM20A, MMP8 | | | | | | | | | |
| RSAD2, IFI27, SIGLEC1, ISG15, | 0.890 | 0.880 | 0.899 | 0.931 | 0.877 | 0.986 | 0.945 | 0.870 | 1 |
| HERC6, FAM20A, RETN, MMP8 | | | | | | | | | |
| IFI27, IFIT1, IFI44L, SIGLEC1, | 0.890 | 0.880 | 0.899 | 0.934 | 0.885 | 0.984 | 0.945 | 0.882 | 1 |
| HERC6, SLC1A2, OLAH, FAM20A | | | | | | | | | |
| RSAD2, IFI27, IFIT1, IFI44L, | 0.890 | 0.881 | 0.898 | 0.927 | 0.869 | 0.984 | 0.945 | 0.886 | 1 |
| SIGLEC1, ISG15, OLAH, FAM20A | | | | | | | | | |
| RSAD2, IFI27, OAS1, SIGLEC1, | 0.890 | 0.880 | 0.899 | 0.934 | 0.883 | 0.986 | 0.945 | 0.880 | 1 |
| SLC1A2, OLAH, FAM20A, MMP8 | | | | | | | | | |
| IFI27, IFIT1, IFI44L, SIGLEC1, | 0.890 | 0.881 | 0.898 | 0.929 | 0.874 | 0.983 | 0.949 | 0.886 | 1 |
| ISG15, IL1R2, FAM20A, RETN | | | | | | | | | |
| IFI27, OAS1, SIGLEC1, ISG15, | 0.889 | 0.880 | 0.899 | 0.926 | 0.869 | 0.983 | 0.946 | 0.882 | 1 |
| IL1R2, OLAH, FAM20A, RETN | | | | | | | | | |
| RSAD2, IFI27, IFI44L, SIGLEC1, | 0.889 | 0.880 | 0.899 | 0.930 | 0.876 | 0.985 | 0.940 | 0.874 | 1 |
| HERC6, OLAH, FAM20A, RETN | | | | | | | | | |
| IFI27, OAS1, IFIT1, IFI44L, | 0.889 | 0.881 | 0.898 | 0.927 | 0.872 | 0.983 | 0.949 | 0.887 | 1 |
| SIGLEC1, IL1R2, FAM20A, RETN | | | | | | | | | |
| IFI27, OAS1, IFIT1, ISG15, HERC6, | 0.889 | 0.880 | 0.899 | 0.926 | 0.870 | 0.981 | 0.933 | 0.863 | 1 |
| OLAH, FAM20A, MMP8 | | | | | | | | | |
| IFI27, OAS1, SIGLEC1, ISG15, | 0.889 | 0.880 | 0.899 | 0.932 | 0.880 | 0.983 | 0.945 | 0.877 | 1 |
| HERC6, IL1R2, FAM20A, RETN | | | | | | | | | |
| IFI27, SIGLEC1, ISG15, HERC6, | 0.889 | 0.880 | 0.899 | 0.928 | 0.873 | 0.983 | 0.948 | 0.886 | 1 |
| IL1R2, OLAH, FAM20A, RETN | | | | | | | | | |
| RSAD2, IFI27, IFI44L, ISG15, | 0.889 | 0.879 | 0.899 | 0.930 | 0.876 | 0.984 | 0.933 | 0.865 | 1 |
| HERC6, OLAH, FAM20A, MMP8 | | | | | | | | | |
| IFI27, OAS1, IFIT1, IFI44L, IL1R2, | 0.889 | 0.880 | 0.899 | 0.927 | 0.873 | 0.982 | 0.933 | 0.863 | 1 |
| OLAH, FAM20A, MMP8 | | | | | | | | | |
| IFI27, OAS1, IFIT1, ISG15, IL1R2, | 0.889 | 0.880 | 0.899 | 0.928 | 0.875 | 0.981 | 0.934 | 0.864 | 1 |
| OLAH, FAM20A, MMP8 | | | | | | | | | |
| IFI27, OAS1, IFIT1, SIGLEC1, | 0.889 | 0.880 | 0.898 | 0.935 | 0.889 | 0.980 | 0.951 | 0.884 | 1 |
| SLC1A2, IL1R2, FAM20A, MMP8 | | | | | | | | | |
| RSAD2, IFI27, SIGLEC1, HERC6, | 0.889 | 0.880 | 0.899 | 0.936 | 0.890 | 0.982 | 0.954 | 0.886 | 1 |
| SLC1A2, IL1R2, FAM20A, MMP8 | | | | | | | | | |
| IFI27, IFI44L, ISG15, IL1R2, | 0.889 | 0.880 | 0.899 | 0.930 | 0.875 | 0.984 | 0.934 | 0.865 | 1 |
| OLAH, FAM20A, RETN, MMP8 | | | | | | | | | |
| RSAD2, IFI27, OAS1, IFI44L, | 0.889 | 0.879 | 0.899 | 0.929 | 0.875 | 0.984 | 0.928 | 0.857 | 1.000 |
| ISG15, HERC6, OLAH, FAM20A | | | | | | | | | |
| RSAD2, IFI27, OAS1, SIGLEC1, | 0.889 | 0.880 | 0.898 | 0.926 | 0.868 | 0.983 | 0.946 | 0.882 | 1 |
| ISG15, IL1R2, OLAH, FAM20A | | | | | | | | | |
| IFI27, OAS1, IFIT1, ISG15, HERC6, | 0.889 | 0.880 | 0.898 | 0.931 | 0.879 | 0.983 | 0.934 | 0.864 | 1 |
| OLAH, FAM20A, RETN | | | | | | | | | |
| IFI27, OAS1, IFI44L, ISG15, IL1R2, | 0.889 | 0.879 | 0.899 | 0.929 | 0.876 | 0.982 | 0.929 | 0.858 | 1 |
| OLAH, FAM20A, MMP8 | | | | | | | | | |
| IFI27, IFIT1, IFI44L, ISG15, | 0.889 | 0.880 | 0.898 | 0.927 | 0.873 | 0.982 | 0.933 | 0.863 | 1 |
| HERC6, OLAH, FAM20A, RETN | | | | | | | | | |
| IFI27, IFI44L, SIGLEC1, ISG15, | 0.889 | 0.880 | 0.898 | 0.931 | 0.878 | 0.984 | 0.942 | 0.872 | 1 |
| HERC6, IL1R2, FAM20A, RETN | | | | | | | | | |
| IFI27, IFIT1, IFI44L, SIGLEC1, | 0.889 | 0.880 | 0.898 | 0.935 | 0.890 | 0.981 | 0.954 | 0.886 | 1 |
| SLC1A2, IL1R2, FAM20A, MMP8 | | | | | | | | | |
| RSAD2, IFI27, IFIT1, IFI44L, | 0.889 | 0.880 | 0.898 | 0.929 | 0.875 | 0.983 | 0.933 | 0.863 | 1 |
| IL1R2, OLAH, FAM20A, MMP8 | | | | | | | | | |
| IFI27, OAS1, IFIT1, IFI44L, | 0.889 | 0.880 | 0.898 | 0.931 | 0.877 | 0.985 | 0.941 | 0.877 | 1 |
| SIGLEC1, SLC1A2, OLAH, FAM20A | | | | | | | | | |
| RSAD2, IFI27, IFI44L, SIGLEC1, | 0.889 | 0.879 | 0.899 | 0.932 | 0.880 | 0.984 | 0.943 | 0.875 | 1 |
| HERC6, IL1R2, FAM20A, RETN | | | | | | | | | |
| RSAD2, IFI27, IFI44L, SIGLEC1, | 0.889 | 0.880 | 0.898 | 0.928 | 0.873 | 0.983 | 0.946 | 0.882 | 1 |
| ISG15, IL1R2, OLAH, FAM20A | | | | | | | | | |
| RSAD2, IFI27, OAS1, IFI44L, | 0.889 | 0.880 | 0.898 | 0.926 | 0.869 | 0.982 | 0.952 | 0.892 | 1 |
| SIGLEC1, IL1R2, OLAH, FAM20A | | | | | | | | | |
| IFI27, OAS1, IFI44L, SIGLEC1, | 0.889 | 0.879 | 0.899 | 0.935 | 0.884 | 0.986 | 0.942 | 0.877 | 1 |
| SLC1A2, OLAH, FAM20A, MMP8 | | | | | | | | | |
| RSAD2, IFI27, IFI44L, ISG15, | 0.889 | 0.880 | 0.898 | 0.929 | 0.875 | 0.983 | 0.929 | 0.857 | 1 |
| HERC6, IL1R2, OLAH, FAM20A | | | | | | | | | |
| IFI27, IFI44L, ISG15, HERC6, | 0.889 | 0.879 | 0.899 | 0.927 | 0.871 | 0.984 | 0.938 | 0.871 | 1 |
| OLAH, FAM20A, RETN, MMP8 | | | | | | | | | |
| RSAD2, IFI27, IFIT1, SIGLEC1, | 0.889 | 0.880 | 0.898 | 0.928 | 0.871 | 0.984 | 0.948 | 0.886 | 1 |
| IL1R2, OLAH, FAM20A, RETN | | | | | | | | | |
| IFI27, OAS1, IFI44L, SIGLEC1, | 0.889 | 0.880 | 0.898 | 0.926 | 0.869 | 0.982 | 0.952 | 0.892 | 1 |
| IL1R2, OLAH, FAM20A, RETN | | | | | | | | | |
| RSAD2, IFI27, OAS1, IFI44L, | 0.889 | 0.879 | 0.899 | 0.928 | 0.874 | 0.983 | 0.929 | 0.857 | 1 |
| IL1R2, OLAH, FAM20A, MMP8 | | | | | | | | | |
| RSAD2, IFI27, SIGLEC1, ISG15, | 0.889 | 0.880 | 0.898 | 0.926 | 0.869 | 0.983 | 0.946 | 0.882 | 1 |
| IL1R2, OLAH, FAM20A, RETN | | | | | | | | | |
| RSAD2, IFI27, IFIT1, ISG15, | 0.889 | 0.879 | 0.899 | 0.928 | 0.874 | 0.982 | 0.934 | 0.866 | 1 |
| HERC6, OLAH, FAM20A, MMP8 | | | | | | | | | |
| IFI27, IFI44L, HERC6, IL1R2, | 0.889 | 0.879 | 0.899 | 0.932 | 0.879 | 0.985 | 0.934 | 0.861 | 1 |
| OLAH, FAM20A, RETN, MMP8 | | | | | | | | | |
| IFI27, IFIT1, ISG15, HERC6, | 0.889 | 0.879 | 0.899 | 0.928 | 0.871 | 0.984 | 0.936 | 0.867 | 1 |
| OLAH, FAM20A, RETN, MMP8 | | | | | | | | | |
| IFI27, OAS1, IFI44L, HERC6, | 0.889 | 0.879 | 0.899 | 0.931 | 0.877 | 0.986 | 0.932 | 0.859 | 1 |
| OLAH, FAM20A, RETN, MMP8 | | | | | | | | | |
| IFI27, OAS1, IFI44L, SIGLEC1, | 0.889 | 0.880 | 0.898 | 0.924 | 0.865 | 0.982 | 0.948 | 0.888 | 1 |
| ISG15, OLAH, FAM20A, RETN | | | | | | | | | |
| RSAD2, IFI27, IFIT1, SIGLEC1, | 0.889 | 0.880 | 0.897 | 0.936 | 0.891 | 0.981 | 0.953 | 0.885 | 1 |
| SLC1A2, IL1R2, FAM20A, MMP8 | | | | | | | | | |
| RSAD2, IFI27, SIGLEC1, ISG15, | 0.889 | 0.879 | 0.898 | 0.931 | 0.879 | 0.983 | 0.946 | 0.879 | 1 |
| HERC6, IL1R2, FAM20A, RETN | | | | | | | | | |
| IFI27, SIGLEC1, HERC6, SLC1A2, | 0.889 | 0.879 | 0.898 | 0.935 | 0.886 | 0.983 | 0.949 | 0.883 | 1 |
| IL1R2, OLAH, FAM20A, MMP8 | | | | | | | | | |
| IFI27, OAS1, IFI44L, SIGLEC1, | 0.889 | 0.879 | 0.899 | 0.927 | 0.870 | 0.985 | 0.957 | 0.902 | 1 |
| OLAH, FAM20A, RETN, MMP8 | | | | | | | | | |
| IFI27, IFIT1, IFI44L, HERC6, | 0.889 | 0.879 | 0.899 | 0.928 | 0.872 | 0.985 | 0.938 | 0.871 | 1 |
| OLAH, FAM20A, RETN, MMP8 | | | | | | | | | |
| RSAD2, IFI27, SIGLEC1, ISG15, | 0.889 | 0.879 | 0.898 | 0.938 | 0.895 | 0.981 | 0.952 | 0.883 | 1 |
| SLC1A2, IL1R2, FAM20A, MMP8 | | | | | | | | | |
| RSAD2, IFI27, ISG15, HERC6, | 0.889 | 0.880 | 0.898 | 0.929 | 0.876 | 0.982 | 0.926 | 0.851 | 1 |
| IL1R2, OLAH, FAM20A, RETN | | | | | | | | | |
| RSAD2, IFI27, SIGLEC1, SLC1A2, | 0.889 | 0.879 | 0.898 | 0.937 | 0.890 | 0.985 | 0.952 | 0.883 | 1 |
| IL1R2, FAM20A, RETN, MMP8 | | | | | | | | | |
| IFI27, OAS1, SIGLEC1, ISG15, | 0.889 | 0.879 | 0.899 | 0.934 | 0.885 | 0.984 | 0.936 | 0.866 | 1 |
| HERC6, SLC1A2, OLAH, FAM20A | | | | | | | | | |
| RSAD2, IFI27, OAS1, SIGLEC1, | 0.888 | 0.879 | 0.898 | 0.929 | 0.875 | 0.983 | 0.937 | 0.863 | 1 |
| ISG15, HERC6, FAM20A, RETN | | | | | | | | | |
| IFI27, IFIT1, SIGLEC1, ISG15, | 0.888 | 0.880 | 0.897 | 0.938 | 0.896 | 0.981 | 0.954 | 0.886 | 1 |
| SLC1A2, IL1R2, FAM20A, MMP8 | | | | | | | | | |
| IFI27, OAS1, IFIT1, IFI44L, | 0.888 | 0.880 | 0.897 | 0.928 | 0.874 | 0.983 | 0.949 | 0.886 | 1 |
| SIGLEC1, ISG15, IL1R2, FAM20A | | | | | | | | | |
| IFI27, OAS1, SIGLEC1, SLC1A2, | 0.888 | 0.879 | 0.898 | 0.936 | 0.890 | 0.982 | 0.951 | 0.883 | 1 |
| IL1R2, FAM20A, RETN, MMP8 | | | | | | | | | |
| RSAD2, IFI27, IFIT1, ISG15, | 0.888 | 0.879 | 0.897 | 0.930 | 0.877 | 0.982 | 0.929 | 0.857 | 1 |
| HERC6, IL1R2, OLAH, FAM20A | | | | | | | | | |
| IFI27, OAS1, IFIT1, HERC6, IL1R2, | 0.888 | 0.879 | 0.898 | 0.931 | 0.879 | 0.982 | 0.929 | 0.855 | 1 |
| OLAH, FAM20A, RETN | | | | | | | | | |
| RSAD2, IFI27, OAS1, IFIT1, ISG15, | 0.888 | 0.879 | 0.898 | 0.928 | 0.874 | 0.983 | 0.928 | 0.856 | 1 |
| HERC6, OLAH, FAM20A | | | | | | | | | |
| IFI27, IFI44L, SIGLEC1, ISG15, | 0.888 | 0.880 | 0.897 | 0.936 | 0.889 | 0.983 | 0.940 | 0.872 | 1 |
| SLC1A2, IL1R2, OLAH, FAM20A | | | | | | | | | |
| RSAD2, IFI27, IFI44L, ISG15, | 0.888 | 0.879 | 0.898 | 0.929 | 0.876 | 0.982 | 0.928 | 0.856 | 1 |
| IL1R2, OLAH, FAM20A, MMP8 | | | | | | | | | |
| RSAD2, IFI27, OAS1, IFI44L, | 0.888 | 0.879 | 0.897 | 0.924 | 0.865 | 0.983 | 0.947 | 0.886 | 1 |
| SIGLEC1, ISG15, OLAH, FAM20A | | | | | | | | | |
| IFI27, IFIT1, IFI44L, SIGLEC1, | 0.888 | 0.880 | 0.897 | 0.932 | 0.881 | 0.983 | 0.942 | 0.878 | 1 |
| SLC1A2, IL1R2, OLAH, FAM20A | | | | | | | | | |
| IFI27, SIGLEC1, ISG15, SLC1A2, | 0.888 | 0.879 | 0.898 | 0.934 | 0.884 | 0.984 | 0.947 | 0.878 | 1 |
| OLAH, FAM20A, RETN, MMP8 | | | | | | | | | |
| IFI27, IFIT1, SIGLEC1, ISG15, | 0.888 | 0.879 | 0.897 | 0.937 | 0.889 | 0.984 | 0.937 | 0.869 | 1 |
| SLC1A2, IL1R2, OLAH, FAM20A | | | | | | | | | |
| IFI27, OAS1, IFI44L, SIGLEC1, | 0.888 | 0.879 | 0.897 | 0.936 | 0.890 | 0.981 | 0.949 | 0.882 | 1 |
| SLC1A2, IL1R2, FAM20A, MMP8 | | | | | | | | | |
| IFI27, OAS1, IFI44L, HERC6, | 0.888 | 0.879 | 0.897 | 0.931 | 0.879 | 0.983 | 0.930 | 0.858 | 1 |
| IL1R2, OLAH, FAM20A, RETN | | | | | | | | | |
| IFI27, IFIT1, SIGLEC1, HERC6, | 0.888 | 0.879 | 0.897 | 0.937 | 0.893 | 0.981 | 0.955 | 0.888 | 1 |
| SLC1A2, IL1R2, FAM20A, MMP8 | | | | | | | | | |
| RSAD2, IFI27, IFIT1, IFI44L, | 0.888 | 0.878 | 0.898 | 0.930 | 0.875 | 0.984 | 0.927 | 0.856 | 0.998 |
| ISG15, HERC6, OLAH, FAM20A | | | | | | | | | |
| IFI27, IFI44L, ISG15, HERC6, | 0.888 | 0.879 | 0.897 | 0.928 | 0.874 | 0.982 | 0.930 | 0.858 | 1 |
| IL1R2, OLAH, FAM20A, RETN | | | | | | | | | |
| IFI27, OAS1, IFIT1, SIGLEC1, | 0.888 | 0.879 | 0.898 | 0.934 | 0.884 | 0.984 | 0.939 | 0.871 | 1 |
| ISG15, SLC1A2, OLAH, FAM20A | | | | | | | | | |
| IFI27, OAS1, IFI44L, ISG15, | 0.888 | 0.879 | 0.898 | 0.930 | 0.877 | 0.983 | 0.934 | 0.864 | 1 |
| HERC6, OLAH, FAM20A, RETN | | | | | | | | | |
| RSAD2, IFI27, OAS1, HERC6, | 0.888 | 0.879 | 0.897 | 0.931 | 0.879 | 0.982 | 0.929 | 0.855 | 1 |
| IL1R2, OLAH, FAM20A, RETN | | | | | | | | | |
| RSAD2, IFI27, IFI44L, HERC6, | 0.888 | 0.878 | 0.898 | 0.931 | 0.876 | 0.985 | 0.929 | 0.857 | 1 |
| OLAH, FAM20A, RETN, MMP8 | | | | | | | | | |
| IFI27, IFIT1, IFI44L, IL1R2, OLAH, | 0.888 | 0.879 | 0.897 | 0.929 | 0.875 | 0.983 | 0.933 | 0.863 | 1 |
| FAM20A, RETN, MMP8 | | | | | | | | | |
| RSAD2, IFI27, IFIT1, ISG15, IL1R2, | 0.888 | 0.879 | 0.897 | 0.929 | 0.876 | 0.982 | 0.935 | 0.866 | 1 |
| OLAH, FAM20A, MMP8 | | | | | | | | | |
| RSAD2, IFI27, IFIT1, IFI44L, | 0.888 | 0.879 | 0.897 | 0.930 | 0.878 | 0.983 | 0.928 | 0.857 | 1.000 |
| HERC6, OLAH, FAM20A, RETN | | | | | | | | | |
| IFI27, OAS1, ISG15, HERC6, | 0.888 | 0.879 | 0.897 | 0.931 | 0.879 | 0.982 | 0.927 | 0.853 | 1 |
| IL1R2, OLAH, FAM20A, RETN | | | | | | | | | |
| RSAD2, IFI27, OAS1, SIGLEC1, | 0.888 | 0.879 | 0.897 | 0.925 | 0.868 | 0.983 | 0.947 | 0.883 | 1 |
| IL1R2, OLAH, FAM20A, RETN | | | | | | | | | |
| RSAD2, IFI27, OAS1, ISG15, | 0.888 | 0.878 | 0.898 | 0.929 | 0.875 | 0.982 | 0.933 | 0.864 | 1 |
| IL1R2, OLAH, FAM20A, MMP8 | | | | | | | | | |
| IFI27, OAS1, SIGLEC1, HERC6, | 0.888 | 0.878 | 0.898 | 0.931 | 0.881 | 0.982 | 0.936 | 0.867 | 1 |
| SLC1A2, IL1R2, OLAH, FAM20A | | | | | | | | | |
| RSAD2, IFI27, IFIT1, SIGLEC1, | 0.888 | 0.878 | 0.898 | 0.935 | 0.887 | 0.983 | 0.938 | 0.872 | 1 |
| HERC6, SLC1A2, OLAH, FAM20A | | | | | | | | | |
| RSAD2, IFI27, IFIT1, IFI44L, | 0.888 | 0.879 | 0.897 | 0.923 | 0.864 | 0.983 | 0.948 | 0.887 | 1 |
| SIGLEC1, OLAH, FAM20A, RETN | | | | | | | | | |
| IFI27, IFIT1, SIGLEC1, HERC6, | 0.888 | 0.879 | 0.897 | 0.936 | 0.888 | 0.983 | 0.941 | 0.876 | 1 |
| SLC1A2, IL1R2, OLAH, FAM20A | | | | | | | | | |
| IFI27, OAS1, IFIT1, HERC6, IL1R2, | 0.888 | 0.878 | 0.897 | 0.930 | 0.877 | 0.983 | 0.928 | 0.847 | 1 |
| FAM20A, RETN, MMP8 | | | | | | | | | |
| RSAD2, IFI27, OAS1, IFIT1, | 0.888 | 0.879 | 0.897 | 0.924 | 0.865 | 0.983 | 0.951 | 0.891 | 1 |
| SIGLEC1, OLAH, FAM20A, RETN | | | | | | | | | |
| RSAD2, IFI27, IFI44L, SIGLEC1, | 0.888 | 0.878 | 0.898 | 0.929 | 0.871 | 0.986 | 0.955 | 0.900 | 1 |
| OLAH, FAM20A, RETN, MMP8 | | | | | | | | | |
| RSAD2, IFI27, IFI44L, SIGLEC1, | 0.888 | 0.879 | 0.897 | 0.935 | 0.890 | 0.981 | 0.953 | 0.885 | 1 |
| SLC1A2, IL1R2, FAM20A, MMP8 | | | | | | | | | |
| IFI27, IFIT1, SIGLEC1, SLC1A2, | 0.888 | 0.879 | 0.897 | 0.939 | 0.893 | 0.985 | 0.954 | 0.886 | 1 |
| IL1R2, FAM20A, RETN, MMP8 | | | | | | | | | |
| IFI27, OAS1, IFIT1, IFI44L, | 0.888 | 0.879 | 0.897 | 0.929 | 0.878 | 0.980 | 0.928 | 0.844 | 1 |
| HERC6, IL1R2, FAM20A, MMP8 | | | | | | | | | |
| RSAD2, IFI27, IFI44L, SIGLEC1, | 0.888 | 0.878 | 0.898 | 0.933 | 0.880 | 0.985 | 0.947 | 0.884 | 1 |
| SLC1A2, OLAH, FAM20A, MMP8 | | | | | | | | | |
| RSAD2, IFI27, IFIT1, IFI44L, | 0.888 | 0.879 | 0.897 | 0.930 | 0.876 | 0.984 | 0.941 | 0.876 | 1 |
| SIGLEC1, SLC1A2, OLAH, FAM20A | | | | | | | | | |
| IFI27, IFIT1, ISG15, IL1R2, OLAH, | 0.888 | 0.878 | 0.897 | 0.927 | 0.871 | 0.982 | 0.934 | 0.864 | 1 |
| FAM20A, RETN, MMP8 | | | | | | | | | |
| IFI27, IFI44L, SIGLEC1, ISG15, | 0.888 | 0.878 | 0.897 | 0.934 | 0.885 | 0.983 | 0.938 | 0.870 | 1 |
| HERC6, SLC1A2, OLAH, FAM20A | | | | | | | | | |
| RSAD2, IFI27, IFIT1, SIGLEC1, | 0.888 | 0.879 | 0.897 | 0.926 | 0.868 | 0.985 | 0.943 | 0.882 | 1 |
| ISG15, OLAH, FAM20A, RETN | | | | | | | | | |
| IFI27, OAS1, IFIT1, SIGLEC1, | 0.888 | 0.878 | 0.897 | 0.927 | 0.873 | 0.981 | 0.954 | 0.903 | 1 |
| HERC6, OLAH, RETN, MMP8 | | | | | | | | | |
| RSAD2, IFI27, HERC6, SLC1A2, | 0.887 | 0.878 | 0.897 | 0.934 | 0.887 | 0.982 | 0.922 | 0.840 | 1 |
| IL1R2, OLAH, FAM20A, MMP8 | | | | | | | | | |
| IFI27, OAS1, IFIT1, SIGLEC1, | 0.887 | 0.878 | 0.897 | 0.936 | 0.891 | 0.980 | 0.941 | 0.872 | 1 |
| HERC6, SLC1A2, IL1R2, FAM20A | | | | | | | | | |
| RSAD2, IFI27, OAS1, SIGLEC1, | 0.887 | 0.878 | 0.897 | 0.935 | 0.889 | 0.982 | 0.949 | 0.882 | 1 |
| SLC1A2, IL1R2, FAM20A, MMP8 | | | | | | | | | |
| RSAD2, IFI27, OAS1, IFIT1, | 0.887 | 0.878 | 0.897 | 0.931 | 0.880 | 0.983 | 0.933 | 0.862 | 1 |
| HERC6, OLAH, FAM20A, RETN | | | | | | | | | |
| IFI27, IFI44L, SIGLEC1, SLC1A2, | 0.887 | 0.878 | 0.896 | 0.935 | 0.886 | 0.983 | 0.955 | 0.887 | 1 |
| IL1R2, FAM20A, RETN, MMP8 | | | | | | | | | |
| IFI27, SIGLEC1, ISG15, HERC6, | 0.887 | 0.878 | 0.897 | 0.937 | 0.894 | 0.981 | 0.955 | 0.887 | 1 |
| SLC1A2, IL1R2, FAM20A, MMP8 | | | | | | | | | |
| IFI27, OAS1, IFI44L, HERC6, | 0.887 | 0.878 | 0.897 | 0.931 | 0.878 | 0.983 | 0.932 | 0.850 | 1 |
| IL1R2, FAM20A, RETN, MMP8 | | | | | | | | | |
| RSAD2, IFI27, OAS1, IFIT1, | 0.887 | 0.879 | 0.896 | 0.929 | 0.874 | 0.983 | 0.951 | 0.890 | 1 |
| IFI44L, SIGLEC1, IL1R2, FAM20A | | | | | | | | | |
| RSAD2, IFI27, OAS1, SIGLEC1, | 0.887 | 0.878 | 0.897 | 0.924 | 0.865 | 0.983 | 0.945 | 0.883 | 1 |
| ISG15, OLAH, FAM20A, RETN | | | | | | | | | |
| IFI27, IFIT1, IFI44L, HERC6, | 0.887 | 0.878 | 0.897 | 0.929 | 0.875 | 0.983 | 0.928 | 0.853 | 1 |
| IL1R2, OLAH, FAM20A, RETN | | | | | | | | | |
| IFI27, OAS1, SIGLEC1, SLC1A2, | 0.887 | 0.877 | 0.897 | 0.932 | 0.879 | 0.985 | 0.951 | 0.892 | 1 |
| OLAH, FAM20A, RETN, MMP8 | | | | | | | | | |
| IFI27, IFIT1, IFI44L, ISG15, | 0.887 | 0.878 | 0.897 | 0.928 | 0.875 | 0.981 | 0.924 | 0.847 | 1 |
| SLC1A2, OLAH, FAM20A, MMP8 | | | | | | | | | |
| IFI27, IFIT1, ISG15, HERC6, IL1R2, | 0.887 | 0.878 | 0.896 | 0.927 | 0.874 | 0.981 | 0.928 | 0.854 | 1 |
| OLAH, FAM20A, RETN | | | | | | | | | |
| IFI27, IFIT1, SIGLEC1, ISG15, | 0.887 | 0.878 | 0.896 | 0.935 | 0.885 | 0.985 | 0.938 | 0.871 | 1 |
| SLC1A2, OLAH, FAM20A, RETN | | | | | | | | | |
| IFI27, OAS1, ISG15, IL1R2, OLAH, | 0.887 | 0.878 | 0.897 | 0.929 | 0.874 | 0.983 | 0.930 | 0.860 | 1 |
| FAM20A, RETN, MMP8 | | | | | | | | | |
| IFI27, OAS1, IFIT1, SIGLEC1, | 0.887 | 0.878 | 0.896 | 0.932 | 0.881 | 0.983 | 0.941 | 0.875 | 1 |
| SLC1A2, IL1R2, OLAH, FAM20A | | | | | | | | | |
| IFI27, OAS1, IFIT1, SIGLEC1, | 0.887 | 0.878 | 0.896 | 0.927 | 0.872 | 0.982 | 0.948 | 0.883 | 1 |
| ISG15, IL1R2, FAM20A, RETN | | | | | | | | | |
| RSAD2, IFI27, OAS1, IFI44L, | 0.887 | 0.878 | 0.897 | 0.930 | 0.877 | 0.982 | 0.933 | 0.862 | 1 |
| HERC6, OLAH, FAM20A, RETN | | | | | | | | | |
| RSAD2, IFI27, IFIT1, SIGLEC1, | 0.887 | 0.878 | 0.896 | 0.935 | 0.885 | 0.985 | 0.938 | 0.871 | 1 |
| ISG15, SLC1A2, OLAH, FAM20A | | | | | | | | | |
| RSAD2, IFI27, IFIT1, IFI44L, | 0.887 | 0.878 | 0.896 | 0.928 | 0.876 | 0.979 | 0.932 | 0.850 | 1 |
| HERC6, IL1R2, FAM20A, MMP8 | | | | | | | | | |
| IFI27, OAS1, SIGLEC1, ISG15, | 0.887 | 0.878 | 0.897 | 0.937 | 0.893 | 0.980 | 0.948 | 0.879 | 1 |
| SLC1A2, IL1R2, FAM20A, MMP8 | | | | | | | | | |
| RSAD2, IFI27, IFIT1, HERC6, | 0.887 | 0.878 | 0.896 | 0.929 | 0.877 | 0.982 | 0.928 | 0.855 | 1 |
| IL1R2, OLAH, FAM20A, RETN | | | | | | | | | |
| RSAD2, IFI27, OAS1, ISG15, | 0.887 | 0.878 | 0.896 | 0.931 | 0.878 | 0.983 | 0.935 | 0.866 | 1 |
| HERC6, OLAH, FAM20A, RETN | | | | | | | | | |
| RSAD2, IFI27, OAS1, HERC6, | 0.887 | 0.877 | 0.897 | 0.930 | 0.879 | 0.980 | 0.922 | 0.841 | 1 |
| SLC1A2, OLAH, FAM20A, MMP8 | | | | | | | | | |
| IFI27, OAS1, IFI44L, SIGLEC1, | 0.887 | 0.878 | 0.896 | 0.930 | 0.877 | 0.983 | 0.948 | 0.883 | 1 |
| ISG15, IL1R2, FAM20A, RETN | | | | | | | | | |
| IFI27, OAS1, IFI44L, SIGLEC1, | 0.887 | 0.877 | 0.897 | 0.932 | 0.881 | 0.983 | 0.938 | 0.870 | 1 |
| ISG15, SLC1A2, OLAH, FAM20A | | | | | | | | | |
| RSAD2, IFI27, OAS1, IFI44L, | 0.887 | 0.878 | 0.896 | 0.929 | 0.874 | 0.983 | 0.939 | 0.866 | 1 |
| SIGLEC1, HERC6, FAM20A, RETN | | | | | | | | | |
| RSAD2, IFI27, SIGLEC1, SLC1A2, | 0.887 | 0.877 | 0.897 | 0.935 | 0.885 | 0.986 | 0.948 | 0.884 | 1 |
| OLAH, FAM20A, RETN, MMP8 | | | | | | | | | |
| IFI27, OAS1, HERC6, SLC1A2, | 0.887 | 0.878 | 0.896 | 0.935 | 0.889 | 0.982 | 0.921 | 0.838 | 1 |
| IL1R2, OLAH, FAM20A, MMP8 | | | | | | | | | |
| IFI27, OAS1, IFI44L, SIGLEC1, | 0.887 | 0.877 | 0.897 | 0.932 | 0.882 | 0.983 | 0.936 | 0.867 | 1 |
| HERC6, SLC1A2, OLAH, FAM20A | | | | | | | | | |
| IFI27, IFIT1, SIGLEC1, SLC1A2, | 0.887 | 0.877 | 0.897 | 0.937 | 0.887 | 0.986 | 0.953 | 0.892 | 1 |
| OLAH, FAM20A, RETN, MMP8 | | | | | | | | | |
| RSAD2, IFI27, IFI44L, HERC6, | 0.887 | 0.877 | 0.897 | 0.929 | 0.876 | 0.982 | 0.929 | 0.857 | 1 |
| IL1R2, OLAH, FAM20A, RETN | | | | | | | | | |
| IFI27, OAS1, ISG15, HERC6, | 0.887 | 0.878 | 0.896 | 0.933 | 0.882 | 0.984 | 0.926 | 0.843 | 1 |
| IL1R2, FAM20A, RETN, MMP8 | | | | | | | | | |
| RSAD2, IFI27, IFI44L, ISG15, | 0.887 | 0.877 | 0.896 | 0.930 | 0.878 | 0.983 | 0.935 | 0.867 | 1 |
| HERC6, OLAH, FAM20A, RETN | | | | | | | | | |
| RSAD2, IFI27, IFIT1, ISG15, | 0.887 | 0.877 | 0.896 | 0.929 | 0.876 | 0.982 | 0.934 | 0.865 | 1 |
| HERC6, OLAH, FAM20A, RETN | | | | | | | | | |
| IFI27, OAS1, IFIT1, IFI44L, | 0.887 | 0.878 | 0.896 | 0.932 | 0.882 | 0.982 | 0.929 | 0.848 | 1 |
| HERC6, IL1R2, FAM20A, RETN | | | | | | | | | |
| RSAD2, IFI27, SIGLEC1, ISG15, | 0.887 | 0.877 | 0.897 | 0.931 | 0.880 | 0.982 | 0.935 | 0.866 | 1 |
| HERC6, SLC1A2, OLAH, FAM20A | | | | | | | | | |
| IFI27, IFIT1, IFI44L, HERC6, | 0.887 | 0.877 | 0.896 | 0.931 | 0.878 | 0.983 | 0.936 | 0.857 | 1 |
| IL1R2, FAM20A, RETN, MMP8 | | | | | | | | | |
| RSAD2, IFI27, OAS1, IL1R2, | 0.887 | 0.877 | 0.896 | 0.929 | 0.876 | 0.983 | 0.932 | 0.861 | 1 |
| OLAH, FAM20A, RETN, MMP8 | | | | | | | | | |
| RSAD2, IFI27, IFI44L, SIGLEC1, | 0.887 | 0.877 | 0.896 | 0.927 | 0.871 | 0.983 | 0.950 | 0.888 | 1 |
| IL1R2, OLAH, FAM20A, RETN | | | | | | | | | |
| RSAD2, IFI27, SIGLEC1, HERC6, | 0.887 | 0.877 | 0.896 | 0.931 | 0.880 | 0.982 | 0.941 | 0.874 | 1 |
| SLC1A2, IL1R2, OLAH, FAM20A | | | | | | | | | |
| IFI27, OAS1, SIGLEC1, HERC6, | 0.887 | 0.877 | 0.896 | 0.927 | 0.873 | 0.981 | 0.955 | 0.905 | 1 |
| IL1R2, OLAH, RETN, MMP8 | | | | | | | | | |
| IFI27, SIGLEC1, ISG15, SLC1A2, | 0.887 | 0.878 | 0.896 | 0.935 | 0.888 | 0.983 | 0.953 | 0.881 | 1 |
| IL1R2, FAM20A, RETN, MMP8 | | | | | | | | | |
| RSAD2, IFI27, OAS1, HERC6, | 0.887 | 0.877 | 0.896 | 0.932 | 0.881 | 0.984 | 0.929 | 0.846 | 1 |
| IL1R2, FAM20A, RETN, MMP8 | | | | | | | | | |
| IFI27, IFI44L, SIGLEC1, HERC6, | 0.887 | 0.877 | 0.896 | 0.934 | 0.887 | 0.980 | 0.954 | 0.886 | 1 |
| SLC1A2, IL1R2, FAM20A, MMP8 | | | | | | | | | |
| RSAD2, IFI27, IFIT1, IFI44L, | 0.887 | 0.878 | 0.895 | 0.927 | 0.873 | 0.982 | 0.948 | 0.883 | 1 |
| SIGLEC1, ISG15, IL1R2, FAM20A | | | | | | | | | |
| RSAD2, IFI27, OAS1, SIGLEC1, | 0.887 | 0.877 | 0.896 | 0.931 | 0.880 | 0.983 | 0.935 | 0.866 | 1 |
| HERC6, SLC1A2, OLAH, FAM20A | | | | | | | | | |
| RSAD2, IFI27, OAS1, IFI44L, | 0.887 | 0.877 | 0.896 | 0.930 | 0.879 | 0.980 | 0.926 | 0.840 | 1 |
| HERC6, IL1R2, FAM20A, MMP8 | | | | | | | | | |
| RSAD2, IFI27, OAS1, IFI44L, | 0.887 | 0.878 | 0.895 | 0.927 | 0.873 | 0.982 | 0.949 | 0.884 | 1 |
| SIGLEC1, ISG15, IL1R2, FAM20A | | | | | | | | | |
| IFI27, IFI44L, SIGLEC1, HERC6, | 0.886 | 0.877 | 0.896 | 0.928 | 0.874 | 0.982 | 0.953 | 0.892 | 1 |
| SLC1A2, OLAH, FAM20A, MMP8 | | | | | | | | | |
| RSAD2, IFI27, IFI44L, SIGLEC1, | 0.886 | 0.877 | 0.896 | 0.924 | 0.865 | 0.982 | 0.945 | 0.882 | 1 |
| ISG15, OLAH, FAM20A, RETN | | | | | | | | | |
| IFI27, IFIT1, IFI44L, SIGLEC1, | 0.886 | 0.877 | 0.896 | 0.923 | 0.864 | 0.983 | 0.946 | 0.872 | 1 |
| ISG15, FAM20A, RETN, MMP8 | | | | | | | | | |
| IFI27, OAS1, IFIT1, IFI44L, | 0.886 | 0.877 | 0.896 | 0.932 | 0.882 | 0.982 | 0.921 | 0.838 | 1 |
| HERC6, SLC1A2, OLAH, FAM20A | | | | | | | | | |
| RSAD2, IFI27, IFI44L, SIGLEC1, | 0.886 | 0.877 | 0.896 | 0.930 | 0.875 | 0.984 | 0.946 | 0.872 | 1 |
| HERC6, FAM20A, RETN, MMP8 | | | | | | | | | |
| IFI27, OAS1, IFIT1, IL1R2, OLAH, | 0.886 | 0.877 | 0.896 | 0.929 | 0.874 | 0.983 | 0.932 | 0.861 | 1 |
| FAM20A, RETN, MMP8 | | | | | | | | | |
| IFI27, OAS1, IFIT1, IFI44L, | 0.886 | 0.878 | 0.895 | 0.920 | 0.863 | 0.977 | 0.941 | 0.883 | 1.000 |
| SIGLEC1, ISG15, HERC6, RETN | | | | | | | | | |
| IFI27, OAS1, SIGLEC1, ISG15, | 0.886 | 0.877 | 0.896 | 0.933 | 0.883 | 0.983 | 0.939 | 0.871 | 1 |
| SLC1A2, IL1R2, OLAH, FAM20A | | | | | | | | | |
| RSAD2, IFI27, OAS1, SIGLEC1, | 0.886 | 0.877 | 0.896 | 0.932 | 0.880 | 0.984 | 0.940 | 0.873 | 1 |
| ISG15, SLC1A2, OLAH, FAM20A | | | | | | | | | |
| RSAD2, IFI27, IFIT1, IL1R2, | 0.886 | 0.877 | 0.896 | 0.930 | 0.876 | 0.984 | 0.932 | 0.861 | 1 |
| OLAH, FAM20A, RETN, MMP8 | | | | | | | | | |
| IFI27, OAS1, SIGLEC1, HERC6, | 0.886 | 0.876 | 0.896 | 0.931 | 0.881 | 0.982 | 0.935 | 0.864 | 1 |
| SLC1A2, OLAH, FAM20A, RETN | | | | | | | | | |
| IFI27, IFI44L, ISG15, HERC6, | 0.886 | 0.876 | 0.896 | 0.931 | 0.878 | 0.983 | 0.936 | 0.857 | 1 |
| IL1R2, FAM20A, RETN, MMP8 | | | | | | | | | |
| IFI27, OAS1, IFI44L, ISG15, | 0.886 | 0.877 | 0.896 | 0.930 | 0.879 | 0.980 | 0.926 | 0.840 | 1 |
| HERC6, IL1R2, FAM20A, MMP8 | | | | | | | | | |
| RSAD2, IFI27, OAS1, IFIT1, | 0.886 | 0.877 | 0.895 | 0.930 | 0.880 | 0.980 | 0.930 | 0.850 | 1 |
| HERC6, IL1R2, FAM20A, MMP8 | | | | | | | | | |
| IFI27, OAS1, IFI44L, SIGLEC1, | 0.886 | 0.877 | 0.895 | 0.926 | 0.872 | 0.980 | 0.950 | 0.897 | 1 |
| HERC6, OLAH, RETN, MMP8 | | | | | | | | | |
| IFI27, OAS1, ISG15, HERC6, | 0.886 | 0.876 | 0.896 | 0.931 | 0.882 | 0.981 | 0.918 | 0.836 | 1 |
| SLC1A2, OLAH, FAM20A, MMP8 | | | | | | | | | |
| IFI27, IFIT1, IFI44L, SIGLEC1, | 0.886 | 0.877 | 0.895 | 0.930 | 0.876 | 0.984 | 0.942 | 0.877 | 1 |
| SLC1A2, OLAH, FAM20A, RETN | | | | | | | | | |
| IFI27, IFIT1, IFI44L, SIGLEC1, | 0.886 | 0.877 | 0.895 | 0.936 | 0.890 | 0.981 | 0.946 | 0.876 | 1 |
| HERC6, SLC1A2, IL1R2, FAM20A | | | | | | | | | |
| RSAD2, IFI27, OAS1, IFIT1, | 0.886 | 0.877 | 0.895 | 0.932 | 0.878 | 0.985 | 0.943 | 0.878 | 1 |
| SIGLEC1, SLC1A2, OLAH, FAM20A | | | | | | | | | |
| RSAD2, IFI27, OAS1, IFIT1, | 0.886 | 0.877 | 0.895 | 0.927 | 0.871 | 0.982 | 0.950 | 0.888 | 1 |
| SIGLEC1, IL1R2, FAM20A, RETN | | | | | | | | | |
| IFI27, OAS1, IFIT1, IFI44L, | 0.886 | 0.878 | 0.895 | 0.924 | 0.872 | 0.977 | 0.940 | 0.882 | 0.998 |
| SIGLEC1, HERC6, OLAH, RETN | | | | | | | | | |
| IFI27, SIGLEC1, SLC1A2, IL1R2, | 0.886 | 0.877 | 0.896 | 0.934 | 0.885 | 0.984 | 0.948 | 0.880 | 1 |
| OLAH, FAM20A, RETN, MMP8 | | | | | | | | | |
| IFI27, OAS1, IFIT1, IFI44L, | 0.886 | 0.877 | 0.895 | 0.920 | 0.860 | 0.980 | 0.943 | 0.874 | 1 |
| SIGLEC1, ISG15, FAM20A, RETN | | | | | | | | | |
| RSAD2, IFI27, OAS1, IFIT1, | 0.886 | 0.876 | 0.896 | 0.924 | 0.865 | 0.982 | 0.938 | 0.874 | 1 |
| IFI44L, OLAH, FAM20A, MMP8 | | | | | | | | | |
| RSAD2, IFI27, IFI44L, SIGLEC1, | 0.886 | 0.877 | 0.895 | 0.931 | 0.879 | 0.983 | 0.938 | 0.869 | 1 |
| ISG15, SLC1A2, OLAH, FAM20A | | | | | | | | | |
| IFI27, IFI44L, SIGLEC1, ISG15, | 0.886 | 0.877 | 0.895 | 0.932 | 0.881 | 0.983 | 0.939 | 0.871 | 1 |
| SLC1A2, OLAH, FAM20A, RETN | | | | | | | | | |
| IFI27, IFIT1, ISG15, SLC1A2, | 0.886 | 0.877 | 0.895 | 0.934 | 0.886 | 0.981 | 0.926 | 0.849 | 1 |
| IL1R2, OLAH, FAM20A, MMP8 | | | | | | | | | |
| RSAD2, IFI27, OAS1, IFIT1, | 0.886 | 0.877 | 0.895 | 0.927 | 0.873 | 0.982 | 0.950 | 0.887 | 1 |
| SIGLEC1, ISG15, IL1R2, FAM20A | | | | | | | | | |
| IFI27, OAS1, SIGLEC1, ISG15, | 0.886 | 0.876 | 0.895 | 0.933 | 0.881 | 0.985 | 0.937 | 0.869 | 1 |
| SLC1A2, OLAH, FAM20A, RETN | | | | | | | | | |
| RSAD2, IFI27, OAS1, IFIT1, | 0.886 | 0.877 | 0.895 | 0.927 | 0.875 | 0.978 | 0.926 | 0.843 | 1 |
| IFI44L, HERC6, IL1R2, FAM20A | | | | | | | | | |
| RSAD2, IFI27, OAS1, SIGLEC1, | 0.886 | 0.876 | 0.895 | 0.928 | 0.872 | 0.983 | 0.949 | 0.882 | 1 |
| ISG15, IL1R2, FAM20A, RETN | | | | | | | | | |
| IFI27, IFI44L, ISG15, HERC6, | 0.886 | 0.876 | 0.896 | 0.931 | 0.880 | 0.983 | 0.922 | 0.841 | 1 |
| SLC1A2, OLAH, FAM20A, MMP8 | | | | | | | | | |
| RSAD2, IFI27, ISG15, HERC6, | 0.886 | 0.877 | 0.895 | 0.932 | 0.881 | 0.983 | 0.930 | 0.850 | 1 |
| IL1R2, FAM20A, RETN, MMP8 | | | | | | | | | |
| IFI27, IFIT1, IFI44L, SLC1A2, | 0.886 | 0.877 | 0.895 | 0.932 | 0.882 | 0.982 | 0.924 | 0.844 | 1 |
| IL1R2, OLAH, FAM20A, MMP8 | | | | | | | | | |
| RSAD2, IFI27, IFIT1, SIGLEC1, | 0.886 | 0.877 | 0.895 | 0.933 | 0.882 | 0.983 | 0.943 | 0.879 | 1 |
| SLC1A2, IL1R2, OLAH, FAM20A | | | | | | | | | |
| IFI27, OAS1, IFI44L, ISG15, | 0.886 | 0.877 | 0.895 | 0.932 | 0.881 | 0.982 | 0.927 | 0.844 | 1 |
| HERC6, IL1R2, FAM20A, RETN | | | | | | | | | |
| RSAD2, IFI27, OAS1, SIGLEC1, | 0.886 | 0.877 | 0.895 | 0.925 | 0.870 | 0.980 | 0.954 | 0.903 | 1 |
| HERC6, OLAH, RETN, MMP8 | | | | | | | | | |
| RSAD2, IFI27, IFIT1, HERC6, | 0.886 | 0.876 | 0.895 | 0.931 | 0.878 | 0.983 | 0.935 | 0.857 | 1 |
| IL1R2, FAM20A, RETN, MMP8 | | | | | | | | | |
| IFI27, OAS1, IFIT1, HERC6, | 0.886 | 0.876 | 0.896 | 0.929 | 0.877 | 0.981 | 0.923 | 0.843 | 1 |
| SLC1A2, OLAH, FAM20A, MMP8 | | | | | | | | | |
| RSAD2, IFI27, OAS1, IFIT1, ISG15, | 0.886 | 0.876 | 0.895 | 0.922 | 0.863 | 0.982 | 0.936 | 0.871 | 1 |
| OLAH, FAM20A, MMP8 | | | | | | | | | |
| RSAD2, IFI27, ISG15, HERC6, | 0.886 | 0.876 | 0.896 | 0.931 | 0.881 | 0.981 | 0.918 | 0.835 | 1 |
| SLC1A2, OLAH, FAM20A, MMP8 | | | | | | | | | |
| RSAD2, IFI27, ISG15, IL1R2, | 0.886 | 0.876 | 0.896 | 0.928 | 0.873 | 0.982 | 0.930 | 0.858 | 1 |
| OLAH, FAM20A, RETN, MMP8 | | | | | | | | | |
| RSAD2, IFI27, IFI44L, ISG15, | 0.886 | 0.876 | 0.895 | 0.928 | 0.876 | 0.979 | 0.926 | 0.840 | 1 |
| HERC6, IL1R2, FAM20A, MMP8 | | | | | | | | | |
| IFI27, IFIT1, IFI44L, ISG15, OLAH, | 0.886 | 0.876 | 0.896 | 0.922 | 0.862 | 0.982 | 0.941 | 0.875 | 1 |
| FAM20A, RETN, MMP8 | | | | | | | | | |
| IFI27, OAS1, IFIT1, SIGLEC1, | 0.886 | 0.877 | 0.894 | 0.925 | 0.872 | 0.978 | 0.958 | 0.910 | 1 |
| HERC6, IL1R2, RETN, MMP8 | | | | | | | | | |
| IFI27, OAS1, IFIT1, IFI44L, ISG15, | 0.886 | 0.876 | 0.896 | 0.921 | 0.861 | 0.981 | 0.936 | 0.870 | 1 |
| OLAH, FAM20A, MMP8 | | | | | | | | | |
| RSAD2, IFI27, IFI44L, SIGLEC1, | 0.886 | 0.876 | 0.895 | 0.928 | 0.874 | 0.982 | 0.939 | 0.864 | 1 |
| ISG15, HERC6, FAM20A, RETN | | | | | | | | | |
| OAS1, IFIT1, SIGLEC1, HERC6, | 0.886 | 0.877 | 0.895 | 0.924 | 0.870 | 0.978 | 0.962 | 0.906 | 1 |
| IL1R2, OLAH, FAM20A, MMP8 | | | | | | | | | |
| IFI27, IFIT1, SIGLEC1, ISG15, | 0.886 | 0.876 | 0.895 | 0.936 | 0.890 | 0.981 | 0.948 | 0.879 | 1 |
| HERC6, SLC1A2, IL1R2, FAM20A | | | | | | | | | |
| RSAD2, IFI27, HERC6, SLC1A2, | 0.886 | 0.876 | 0.895 | 0.933 | 0.884 | 0.983 | 0.924 | 0.844 | 1 |
| OLAH, FAM20A, RETN, MMP8 | | | | | | | | | |
| IFI27, OAS1, IFIT1, IFI44L, ISG15, | 0.886 | 0.877 | 0.894 | 0.928 | 0.877 | 0.979 | 0.925 | 0.841 | 1 |
| HERC6, IL1R2, FAM20A | | | | | | | | | |
| RSAD2, IFI27, SIGLEC1, ISG15, | 0.886 | 0.876 | 0.895 | 0.934 | 0.885 | 0.983 | 0.938 | 0.869 | 1 |
| SLC1A2, IL1R2, OLAH, FAM20A | | | | | | | | | |
| IFI27, IFI44L, SIGLEC1, SLC1A2, | 0.886 | 0.876 | 0.895 | 0.931 | 0.878 | 0.984 | 0.949 | 0.883 | 1 |
| OLAH, FAM20A, RETN, MMP8 | | | | | | | | | |
| RSAD2, IFI27, OAS1, IFI44L, | 0.886 | 0.877 | 0.894 | 0.927 | 0.876 | 0.979 | 0.925 | 0.840 | 1 |
| ISG15, HERC6, IL1R2, FAM20A | | | | | | | | | |
| IFI27, IFIT1, SIGLEC1, SLC1A2, | 0.885 | 0.876 | 0.895 | 0.933 | 0.883 | 0.983 | 0.943 | 0.879 | 1 |
| IL1R2, OLAH, FAM20A, RETN | | | | | | | | | |
| IFI27, OAS1, IFIT1, SIGLEC1, | 0.885 | 0.877 | 0.894 | 0.924 | 0.870 | 0.978 | 0.946 | 0.892 | 1.000 |
| HERC6, IL1R2, OLAH, RETN | | | | | | | | | |
| IFI27, OAS1, IFIT1, IFI44L, ISG15, | 0.885 | 0.876 | 0.895 | 0.923 | 0.866 | 0.981 | 0.929 | 0.850 | 1 |
| HERC6, FAM20A, RETN | | | | | | | | | |
| IFI27, OAS1, SIGLEC1, ISG15, | 0.885 | 0.876 | 0.895 | 0.926 | 0.871 | 0.981 | 0.957 | 0.905 | 1 |
| HERC6, OLAH, RETN, MMP8 | | | | | | | | | |
| RSAD2, IFI27, IFI44L, SIGLEC1, | 0.885 | 0.876 | 0.895 | 0.931 | 0.880 | 0.983 | 0.939 | 0.871 | 1 |
| HERC6, SLC1A2, OLAH, FAM20A | | | | | | | | | |
| IFI27, IFIT1, IFI44L, SIGLEC1, | 0.885 | 0.877 | 0.894 | 0.933 | 0.885 | 0.980 | 0.943 | 0.874 | 1 |
| ISG15, SLC1A2, IL1R2, FAM20A | | | | | | | | | |
| OAS1, IFIT1, IFI44L, SIGLEC1, | 0.885 | 0.877 | 0.894 | 0.924 | 0.870 | 0.978 | 0.962 | 0.906 | 1 |
| HERC6, IL1R2, FAM20A, MMP8 | | | | | | | | | |
| IFI27, IFIT1, IFI44L, SIGLEC1, | 0.885 | 0.876 | 0.894 | 0.926 | 0.872 | 0.981 | 0.955 | 0.907 | 1 |
| HERC6, OLAH, RETN, MMP8 | | | | | | | | | |
| IFI27, OAS1, IFIT1, SIGLEC1, | 0.885 | 0.876 | 0.894 | 0.925 | 0.872 | 0.978 | 0.947 | 0.892 | 1 |
| ISG15, HERC6, OLAH, RETN | | | | | | | | | |
| RSAD2, IFI27, OAS1, IFI44L, | 0.885 | 0.876 | 0.895 | 0.922 | 0.862 | 0.982 | 0.951 | 0.893 | 1 |
| SIGLEC1, OLAH, FAM20A, RETN | | | | | | | | | |
| IFI27, OAS1, IFIT1, SIGLEC1, | 0.885 | 0.876 | 0.895 | 0.931 | 0.878 | 0.984 | 0.942 | 0.878 | 1 |
| SLC1A2, OLAH, FAM20A, RETN | | | | | | | | | |
| RSAD2, IFI27, IFIT1, IFI44L, | 0.885 | 0.876 | 0.895 | 0.927 | 0.871 | 0.982 | 0.949 | 0.886 | 1 |
| SIGLEC1, IL1R2, FAM20A, RETN | | | | | | | | | |
| RSAD2, IFI27, OAS1, IFI44L, | 0.885 | 0.875 | 0.896 | 0.923 | 0.865 | 0.982 | 0.936 | 0.870 | 1 |
| ISG15, OLAH, FAM20A, MMP8 | | | | | | | | | |
| IFI27, OAS1, IFIT1, ISG15, HERC6, | 0.885 | 0.876 | 0.894 | 0.932 | 0.881 | 0.982 | 0.927 | 0.845 | 1 |
| IL1R2, FAM20A, RETN | | | | | | | | | |
| RSAD2, IFI27, OAS1, HERC6, | 0.885 | 0.876 | 0.895 | 0.934 | 0.886 | 0.981 | 0.918 | 0.833 | 1 |
| SLC1A2, IL1R2, OLAH, FAM20A | | | | | | | | | |
| IFI27, SIGLEC1, ISG15, HERC6, | 0.885 | 0.876 | 0.895 | 0.933 | 0.885 | 0.982 | 0.938 | 0.870 | 1 |
| SLC1A2, IL1R2, OLAH, FAM20A | | | | | | | | | |
| IFI27, ISG15, HERC6, SLC1A2, | 0.885 | 0.876 | 0.895 | 0.935 | 0.889 | 0.981 | 0.922 | 0.841 | 1 |
| IL1R2, OLAH, FAM20A, MMP8 | | | | | | | | | |
| RSAD2, IFI27, IFIT1, SIGLEC1, | 0.885 | 0.876 | 0.894 | 0.928 | 0.873 | 0.983 | 0.949 | 0.884 | 1 |
| ISG15, IL1R2, FAM20A, RETN | | | | | | | | | |
| IFI27, IFI44L, ISG15, SLC1A2, | 0.885 | 0.875 | 0.895 | 0.930 | 0.877 | 0.983 | 0.924 | 0.844 | 1 |
| OLAH, FAM20A, RETN, MMP8 | | | | | | | | | |
| IFI27, IFIT1, IFI44L, HERC6, | 0.885 | 0.876 | 0.895 | 0.931 | 0.880 | 0.982 | 0.925 | 0.844 | 1 |
| SLC1A2, OLAH, FAM20A, MMP8 | | | | | | | | | |
| RSAD2, IFI27, IFIT1, SLC1A2, | 0.885 | 0.876 | 0.894 | 0.931 | 0.882 | 0.981 | 0.924 | 0.845 | 1 |
| IL1R2, OLAH, FAM20A, MMP8 | | | | | | | | | |
| IFI27, IFIT1, SIGLEC1, HERC6, | 0.885 | 0.875 | 0.895 | 0.936 | 0.887 | 0.985 | 0.942 | 0.877 | 1 |
| SLC1A2, OLAH, FAM20A, RETN | | | | | | | | | |
| IFI27, IFI44L, SIGLEC1, ISG15, | 0.885 | 0.875 | 0.895 | 0.926 | 0.868 | 0.984 | 0.949 | 0.876 | 1 |
| HERC6, FAM20A, RETN, MMP8 | | | | | | | | | |
| IFI27, OAS1, IFI44L, SIGLEC1, | 0.885 | 0.876 | 0.895 | 0.934 | 0.889 | 0.980 | 0.943 | 0.874 | 1 |
| HERC6, SLC1A2, IL1R2, FAM20A | | | | | | | | | |
| RSAD2, IFI27, SIGLEC1, HERC6, | 0.885 | 0.876 | 0.894 | 0.925 | 0.871 | 0.979 | 0.954 | 0.905 | 1 |
| IL1R2, OLAH, RETN, MMP8 | | | | | | | | | |
| IFI27, IFI44L, SIGLEC1, HERC6, | 0.885 | 0.876 | 0.895 | 0.933 | 0.882 | 0.983 | 0.947 | 0.881 | 1 |
| SLC1A2, IL1R2, OLAH, FAM20A | | | | | | | | | |
| RSAD2, IFI27, IFI44L, ISG15, | 0.885 | 0.875 | 0.895 | 0.931 | 0.878 | 0.983 | 0.924 | 0.848 | 1.000 |
| SLC1A2, OLAH, FAM20A, MMP8 | | | | | | | | | |
| IFI27, SIGLEC1, ISG15, SLC1A2, | 0.885 | 0.876 | 0.894 | 0.933 | 0.884 | 0.982 | 0.938 | 0.870 | 1 |
| IL1R2, OLAH, FAM20A, RETN | | | | | | | | | |
| IFI27, IFI44L, SIGLEC1, HERC6, | 0.885 | 0.876 | 0.894 | 0.926 | 0.872 | 0.979 | 0.953 | 0.904 | 1 |
| IL1R2, OLAH, RETN, MMP8 | | | | | | | | | |
| IFI27, OAS1, IFIT1, ISG15, HERC6, | 0.885 | 0.876 | 0.894 | 0.929 | 0.878 | 0.980 | 0.929 | 0.848 | 1 |
| IL1R2, FAM20A, MMP8 | | | | | | | | | |
| IFI27, OAS1, IFIT1, SIGLEC1, | 0.885 | 0.876 | 0.894 | 0.931 | 0.882 | 0.981 | 0.937 | 0.866 | 1 |
| HERC6, SLC1A2, FAM20A, RETN | | | | | | | | | |
| IFI27, OAS1, IFIT1, SIGLEC1, | 0.885 | 0.875 | 0.895 | 0.923 | 0.863 | 0.982 | 0.946 | 0.875 | 1 |
| ISG15, FAM20A, RETN, MMP8 | | | | | | | | | |
| IFI27, OAS1, IFI44L, ISG15, | 0.885 | 0.875 | 0.895 | 0.933 | 0.884 | 0.982 | 0.918 | 0.836 | 1 |
| HERC6, SLC1A2, OLAH, FAM20A | | | | | | | | | |
| IFI27, OAS1, IFI44L, HERC6, | 0.885 | 0.875 | 0.895 | 0.930 | 0.878 | 0.981 | 0.923 | 0.843 | 1 |
| SLC1A2, OLAH, FAM20A, MMP8 | | | | | | | | | |
| RSAD2, IFI27, ISG15, SLC1A2, | 0.885 | 0.876 | 0.894 | 0.936 | 0.890 | 0.982 | 0.917 | 0.834 | 1 |
| IL1R2, OLAH, FAM20A, MMP8 | | | | | | | | | |
| IFI27, IFI44L, SLC1A2, IL1R2, | 0.885 | 0.875 | 0.894 | 0.935 | 0.886 | 0.984 | 0.925 | 0.846 | 1 |
| OLAH, FAM20A, RETN, MMP8 | | | | | | | | | |
| RSAD2, IFI27, IFI44L, SLC1A2, | 0.885 | 0.875 | 0.895 | 0.933 | 0.883 | 0.983 | 0.928 | 0.853 | 1 |
| IL1R2, OLAH, FAM20A, MMP8 | | | | | | | | | |
| IFI27, IFIT1, IFI44L, SIGLEC1, | 0.885 | 0.876 | 0.894 | 0.924 | 0.870 | 0.978 | 0.946 | 0.893 | 0.998 |
| ISG15, HERC6, OLAH, RETN | | | | | | | | | |
| IFI27, OAS1, HERC6, SLC1A2, | 0.885 | 0.875 | 0.895 | 0.933 | 0.884 | 0.982 | 0.921 | 0.837 | 1 |
| OLAH, FAM20A, RETN, MMP8 | | | | | | | | | |
| RSAD2, IFI27, IFI44L, SIGLEC1, | 0.885 | 0.875 | 0.894 | 0.928 | 0.874 | 0.982 | 0.947 | 0.880 | 1 |
| ISG15, IL1R2, FAM20A, RETN | | | | | | | | | |
| RSAD2, IFI27, IFI44L, IL1R2, | 0.885 | 0.875 | 0.895 | 0.930 | 0.877 | 0.984 | 0.930 | 0.859 | 1 |
| OLAH, FAM20A, RETN, MMP8 | | | | | | | | | |
| IFI27, OAS1, IFIT1, IFI44L, | 0.885 | 0.876 | 0.894 | 0.933 | 0.885 | 0.981 | 0.945 | 0.877 | 1 |
| SIGLEC1, SLC1A2, IL1R2, FAM20A | | | | | | | | | |
| RSAD2, IFI27, OAS1, IFI44L, | 0.885 | 0.875 | 0.894 | 0.928 | 0.875 | 0.982 | 0.948 | 0.883 | 1 |
| SIGLEC1, IL1R2, FAM20A, RETN | | | | | | | | | |
| IFI27, OAS1, IFIT1, IFI44L, | 0.885 | 0.875 | 0.895 | 0.923 | 0.863 | 0.982 | 0.948 | 0.881 | 1 |
| SIGLEC1, FAM20A, RETN, MMP8 | | | | | | | | | |
| IFI27, IFI44L, SIGLEC1, ISG15, | 0.885 | 0.876 | 0.894 | 0.924 | 0.868 | 0.979 | 0.949 | 0.896 | 1 |
| HERC6, OLAH, RETN, MMP8 | | | | | | | | | |
| RSAD2, IFI27, IFIT1, IFI44L, | 0.885 | 0.876 | 0.894 | 0.927 | 0.876 | 0.978 | 0.929 | 0.847 | 1 |
| ISG15, HERC6, IL1R2, FAM20A | | | | | | | | | |
| RSAD2, IFI27, OAS1, SIGLEC1, | 0.885 | 0.875 | 0.894 | 0.930 | 0.878 | 0.982 | 0.941 | 0.876 | 1 |
| SLC1A2, IL1R2, OLAH, FAM20A | | | | | | | | | |
| RSAD2, IFI27, OAS1, IFIT1, | 0.885 | 0.876 | 0.893 | 0.920 | 0.862 | 0.978 | 0.943 | 0.885 | 1 |
| SIGLEC1, ISG15, HERC6, RETN | | | | | | | | | |
| IFI27, OAS1, IFIT1, SIGLEC1, | 0.885 | 0.876 | 0.893 | 0.920 | 0.863 | 0.978 | 0.945 | 0.887 | 1 |
| ISG15, HERC6, RETN, MMP8 | | | | | | | | | |
| RSAD2, IFI27, OAS1, ISG15, | 0.885 | 0.876 | 0.894 | 0.931 | 0.881 | 0.980 | 0.923 | 0.835 | 1 |
| HERC6, IL1R2, FAM20A, MMP8 | | | | | | | | | |
| IFI27, IFIT1, IFI44L, ISG15, | 0.885 | 0.875 | 0.894 | 0.930 | 0.879 | 0.980 | 0.935 | 0.855 | 1 |
| HERC6, IL1R2, FAM20A, MMP8 | | | | | | | | | |
| RSAD2, IFI27, IFIT1, IFI44L, | 0.885 | 0.876 | 0.893 | 0.922 | 0.863 | 0.981 | 0.942 | 0.872 | 1 |
| SIGLEC1, ISG15, HERC6, FAM20A | | | | | | | | | |
| RSAD2, IFI27, IFI44L, HERC6, | 0.885 | 0.875 | 0.895 | 0.933 | 0.884 | 0.983 | 0.925 | 0.848 | 1 |
| SLC1A2, OLAH, FAM20A, MMP8 | | | | | | | | | |
| RSAD2, IFI27, IFIT1, SIGLEC1, | 0.885 | 0.875 | 0.894 | 0.935 | 0.890 | 0.981 | 0.946 | 0.876 | 1 |
| HERC6, SLC1A2, IL1R2, FAM20A | | | | | | | | | |
| IFI27, IFIT1, IFI44L, SIGLEC1, | 0.885 | 0.876 | 0.894 | 0.920 | 0.861 | 0.980 | 0.937 | 0.862 | 1 |
| ISG15, HERC6, FAM20A, MMP8 | | | | | | | | | |
| IFI27, OAS1, IFI44L, IL1R2, OLAH, | 0.885 | 0.875 | 0.895 | 0.931 | 0.877 | 0.984 | 0.934 | 0.864 | 1 |
| FAM20A, RETN, MMP8 | | | | | | | | | |
| IFI27, OAS1, IFI44L, ISG15, | 0.885 | 0.874 | 0.895 | 0.930 | 0.877 | 0.983 | 0.924 | 0.847 | 1 |
| SLC1A2, OLAH, FAM20A, MMP8 | | | | | | | | | |
| RSAD2, IFI27, IFIT1, SIGLEC1, | 0.885 | 0.876 | 0.894 | 0.924 | 0.869 | 0.980 | 0.951 | 0.900 | 1 |
| HERC6, OLAH, RETN, MMP8 | | | | | | | | | |
| IFI27, OAS1, IFIT1, SLC1A2, | 0.885 | 0.875 | 0.894 | 0.931 | 0.881 | 0.982 | 0.924 | 0.845 | 1 |
| IL1R2, OLAH, FAM20A, MMP8 | | | | | | | | | |
| IFI27, OAS1, IFIT1, HERC6, IL1R2, | 0.885 | 0.876 | 0.894 | 0.924 | 0.868 | 0.979 | 0.945 | 0.885 | 1 |
| OLAH, RETN, MMP8 | | | | | | | | | |
| RSAD2, IFI27, OAS1, SLC1A2, | 0.885 | 0.876 | 0.894 | 0.930 | 0.881 | 0.980 | 0.924 | 0.844 | 1 |
| IL1R2, OLAH, FAM20A, MMP8 | | | | | | | | | |
| RSAD2, IFI27, IFI44L, HERC6, | 0.885 | 0.874 | 0.895 | 0.929 | 0.877 | 0.982 | 0.935 | 0.856 | 1 |
| IL1R2, FAM20A, RETN, MMP8 | | | | | | | | | |
| RSAD2, IFI27, IFI44L, SIGLEC1, | 0.885 | 0.876 | 0.894 | 0.926 | 0.871 | 0.980 | 0.952 | 0.901 | 1 |
| HERC6, OLAH, RETN, MMP8 | | | | | | | | | |
| IFIT1, IFI44L, SIGLEC1, ISG15, | 0.885 | 0.876 | 0.894 | 0.923 | 0.868 | 0.978 | 0.963 | 0.907 | 1 |
| HERC6, IL1R2, FAM20A, MMP8 | | | | | | | | | |
| IFI27, OAS1, IFI44L, SIGLEC1, | 0.885 | 0.875 | 0.894 | 0.930 | 0.877 | 0.982 | 0.943 | 0.879 | 1 |
| SLC1A2, IL1R2, OLAH, FAM20A | | | | | | | | | |
| IFI27, OAS1, IFIT1, IFI44L, | 0.885 | 0.875 | 0.894 | 0.932 | 0.879 | 0.985 | 0.925 | 0.848 | 1 |
| SLC1A2, OLAH, FAM20A, MMP8 | | | | | | | | | |
| IFI27, OAS1, IFIT1, IFI44L, ISG15, | 0.885 | 0.876 | 0.893 | 0.924 | 0.868 | 0.981 | 0.933 | 0.863 | 1 |
| IL1R2, OLAH, FAM20A | | | | | | | | | |
| IFI27, IFIT1, HERC6, SLC1A2, | 0.885 | 0.875 | 0.894 | 0.933 | 0.884 | 0.981 | 0.924 | 0.843 | 1 |
| IL1R2, OLAH, FAM20A, MMP8 | | | | | | | | | |
| IFI27, OAS1, SIGLEC1, ISG15, | 0.885 | 0.875 | 0.894 | 0.935 | 0.889 | 0.980 | 0.942 | 0.873 | 1 |
| HERC6, SLC1A2, IL1R2, FAM20A | | | | | | | | | |
| RSAD2, IFI27, OAS1, SIGLEC1, | 0.885 | 0.875 | 0.894 | 0.933 | 0.886 | 0.979 | 0.943 | 0.874 | 1 |
| HERC6, SLC1A2, IL1R2, FAM20A | | | | | | | | | |
| RSAD2, IFI27, OAS1, ISG15, | 0.885 | 0.875 | 0.894 | 0.932 | 0.883 | 0.981 | 0.915 | 0.829 | 1 |
| HERC6, SLC1A2, OLAH, FAM20A | | | | | | | | | |
| IFI27, OAS1, IFIT1, IFI44L, | 0.885 | 0.876 | 0.893 | 0.922 | 0.865 | 0.980 | 0.939 | 0.868 | 1 |
| SIGLEC1, ISG15, HERC6, FAM20A | | | | | | | | | |
| IFIT1, IFI44L, SIGLEC1, HERC6, | 0.885 | 0.875 | 0.894 | 0.924 | 0.870 | 0.978 | 0.963 | 0.909 | 1 |
| IL1R2, OLAH, FAM20A, MMP8 | | | | | | | | | |
| IFI27, IFI44L, SIGLEC1, ISG15, | 0.885 | 0.875 | 0.894 | 0.931 | 0.884 | 0.979 | 0.948 | 0.879 | 1 |
| HERC6, SLC1A2, IL1R2, FAM20A | | | | | | | | | |
| IFI27, IFIT1, IFI44L, ISG15, | 0.885 | 0.875 | 0.894 | 0.933 | 0.883 | 0.983 | 0.917 | 0.835 | 1.000 |
| HERC6, SLC1A2, OLAH, FAM20A | | | | | | | | | |
| RSAD2, IFI27, OAS1, IFIT1, | 0.885 | 0.875 | 0.894 | 0.924 | 0.867 | 0.980 | 0.945 | 0.886 | 1 |
| HERC6, OLAH, RETN, MMP8 | | | | | | | | | |
| RSAD2, IFI27, OAS1, IFI44L, | 0.885 | 0.875 | 0.894 | 0.924 | 0.867 | 0.981 | 0.928 | 0.856 | 1 |
| ISG15, IL1R2, OLAH, FAM20A | | | | | | | | | |
| IFI27, IFIT1, IFI44L, ISG15, IL1R2, | 0.884 | 0.876 | 0.893 | 0.924 | 0.867 | 0.981 | 0.932 | 0.861 | 1 |
| OLAH, FAM20A, RETN | | | | | | | | | |
| RSAD2, IFI27, OAS1, IFIT1, | 0.884 | 0.876 | 0.893 | 0.926 | 0.873 | 0.979 | 0.947 | 0.891 | 1 |
| SIGLEC1, HERC6, OLAH, RETN | | | | | | | | | |
| RSAD2, IFI27, IFIT1, HERC6, | 0.884 | 0.875 | 0.894 | 0.930 | 0.879 | 0.981 | 0.925 | 0.848 | 1 |
| SLC1A2, OLAH, FAM20A, MMP8 | | | | | | | | | |
| RSAD2, IFI27, SIGLEC1, ISG15, | 0.884 | 0.875 | 0.894 | 0.929 | 0.876 | 0.982 | 0.935 | 0.865 | 1 |
| SLC1A2, OLAH, FAM20A, RETN | | | | | | | | | |
| RSAD2, IFI27, OAS1, IFIT1, | 0.884 | 0.876 | 0.893 | 0.924 | 0.866 | 0.981 | 0.939 | 0.868 | 1 |
| IFI44L, SIGLEC1, HERC6, FAM20A | | | | | | | | | |
| IFI27, OAS1, IFIT1, ISG15, | 0.884 | 0.875 | 0.894 | 0.927 | 0.873 | 0.981 | 0.925 | 0.849 | 1 |
| SLC1A2, OLAH, FAM20A, MMP8 | | | | | | | | | |
| RSAD2, OAS1, IFIT1, SIGLEC1, | 0.884 | 0.876 | 0.893 | 0.923 | 0.869 | 0.977 | 0.962 | 0.906 | 1 |
| HERC6, IL1R2, FAM20A, MMP8 | | | | | | | | | |
| IFI27, OAS1, ISG15, HERC6, | 0.884 | 0.875 | 0.894 | 0.924 | 0.869 | 0.980 | 0.945 | 0.885 | 1 |
| IL1R2, OLAH, RETN, MMP8 | | | | | | | | | |
| IFI27, IFIT1, SIGLEC1, ISG15, | 0.884 | 0.875 | 0.894 | 0.924 | 0.869 | 0.980 | 0.954 | 0.906 | 1 |
| HERC6, OLAH, RETN, MMP8 | | | | | | | | | |
| IFI27, IFIT1, IFI44L, ISG15, | 0.884 | 0.875 | 0.893 | 0.931 | 0.880 | 0.982 | 0.929 | 0.849 | 1 |
| HERC6, IL1R2, FAM20A, RETN | | | | | | | | | |
| IFI27, IFIT1, SIGLEC1, HERC6, | 0.884 | 0.875 | 0.894 | 0.929 | 0.875 | 0.982 | 0.950 | 0.878 | 1 |
| SLC1A2, FAM20A, RETN, MMP8 | | | | | | | | | |
| RSAD2, IFI27, IFIT1, IFI44L, | 0.884 | 0.874 | 0.894 | 0.922 | 0.863 | 0.982 | 0.937 | 0.872 | 1 |
| ISG15, OLAH, FAM20A, MMP8 | | | | | | | | | |
| RSAD2, IFI27, OAS1, IFI44L, | 0.884 | 0.876 | 0.893 | 0.924 | 0.870 | 0.978 | 0.942 | 0.883 | 1 |
| SIGLEC1, HERC6, OLAH, RETN | | | | | | | | | |
| RSAD2, IFI27, OAS1, IFIT1, | 0.884 | 0.875 | 0.893 | 0.926 | 0.869 | 0.983 | 0.930 | 0.858 | 1 |
| IFI44L, IL1R2, OLAH, FAM20A | | | | | | | | | |
| IFI27, OAS1, IFI44L, SIGLEC1, | 0.884 | 0.876 | 0.893 | 0.928 | 0.880 | 0.975 | 0.946 | 0.895 | 0.996 |
| HERC6, IL1R2, OLAH, MMP8 | | | | | | | | | |
| IFI27, OAS1, IFIT1, IFI44L, | 0.884 | 0.876 | 0.893 | 0.923 | 0.867 | 0.980 | 0.937 | 0.865 | 1 |
| SIGLEC1, HERC6, FAM20A, MMP8 | | | | | | | | | |
| IFI27, OAS1, IFIT1, ISG15, HERC6, | 0.884 | 0.875 | 0.894 | 0.924 | 0.868 | 0.980 | 0.945 | 0.886 | 1 |
| OLAH, RETN, MMP8 | | | | | | | | | |
| RSAD2, IFI27, IFIT1, ISG15, | 0.884 | 0.875 | 0.894 | 0.927 | 0.872 | 0.981 | 0.926 | 0.851 | 1 |
| SLC1A2, OLAH, FAM20A, MMP8 | | | | | | | | | |
| IFI27, IFIT1, ISG15, HERC6, | 0.884 | 0.874 | 0.894 | 0.930 | 0.879 | 0.981 | 0.922 | 0.842 | 1 |
| SLC1A2, OLAH, FAM20A, MMP8 | | | | | | | | | |
| RSAD2, IFI27, OAS1, IFI44L, | 0.884 | 0.875 | 0.894 | 0.929 | 0.873 | 0.984 | 0.941 | 0.877 | 1 |
| SIGLEC1, SLC1A2, OLAH, FAM20A | | | | | | | | | |
| IFI27, OAS1, IFIT1, ISG15, IL1R2, | 0.884 | 0.875 | 0.893 | 0.924 | 0.868 | 0.981 | 0.929 | 0.859 | 1.000 |
| OLAH, FAM20A, RETN | | | | | | | | | |
| IFI27, IFI44L, HERC6, SLC1A2, | 0.884 | 0.874 | 0.894 | 0.933 | 0.885 | 0.982 | 0.927 | 0.848 | 1 |
| IL1R2, OLAH, FAM20A, MMP8 | | | | | | | | | |
| IFI27, IFI44L, SIGLEC1, SLC1A2, | 0.884 | 0.875 | 0.894 | 0.930 | 0.878 | 0.982 | 0.947 | 0.882 | 1 |
| IL1R2, OLAH, FAM20A, RETN | | | | | | | | | |
| RSAD2, IFI27, OAS1, IFIT1, | 0.884 | 0.875 | 0.893 | 0.930 | 0.879 | 0.981 | 0.929 | 0.848 | 1 |
| HERC6, IL1R2, FAM20A, RETN | | | | | | | | | |
| IFI27, OAS1, IFIT1, ISG15, HERC6, | 0.884 | 0.874 | 0.894 | 0.932 | 0.882 | 0.981 | 0.917 | 0.835 | 1.00 |
| SLC1A2, OLAH, FAM20A | | | | | | | | | |
| RSAD2, IFI27, OAS1, IFIT1, | 0.884 | 0.876 | 0.893 | 0.920 | 0.861 | 0.978 | 0.942 | 0.881 | 1 |
| IFI44L, SIGLEC1, HERC6, RETN | | | | | | | | | |
| IFI27, IFIT1, SIGLEC1, HERC6, | 0.884 | 0.875 | 0.893 | 0.926 | 0.872 | 0.980 | 0.955 | 0.907 | 1 |
| IL1R2, OLAH, RETN, MMP8 | | | | | | | | | |
| RSAD2, IFI27, OAS1, IFIT1, ISG15, | 0.884 | 0.875 | 0.893 | 0.924 | 0.868 | 0.981 | 0.929 | 0.859 | 1.000 |
| IL1R2, OLAH, FAM20A | | | | | | | | | |
| RSAD2, IFI27, IFIT1, IFI44L, | 0.884 | 0.875 | 0.894 | 0.930 | 0.876 | 0.983 | 0.928 | 0.854 | 1 |
| SLC1A2, OLAH, FAM20A, MMP8 | | | | | | | | | |
| RSAD2, IFI27, OAS1, IFI44L, | 0.884 | 0.874 | 0.894 | 0.931 | 0.880 | 0.981 | 0.929 | 0.848 | 1 |
| HERC6, IL1R2, FAM20A, RETN | | | | | | | | | |
| RSAD2, IFI27, OAS1, IFIT1, OLAH, | 0.884 | 0.874 | 0.894 | 0.924 | 0.866 | 0.983 | 0.941 | 0.877 | 1 |
| FAM20A, RETN, MMP8 | | | | | | | | | |
| IFIT1, SIGLEC1, ISG15, HERC6, | 0.884 | 0.875 | 0.893 | 0.922 | 0.868 | 0.977 | 0.962 | 0.906 | 1 |
| IL1R2, OLAH, FAM20A, MMP8 | | | | | | | | | |
| IFI27, IFIT1, IFI44L, SIGLEC1, | 0.884 | 0.875 | 0.893 | 0.929 | 0.878 | 0.981 | 0.941 | 0.867 | 1 |
| HERC6, SLC1A2, FAM20A, RETN | | | | | | | | | |
| RSAD2, IFI27, IFIT1, IFI44L, | 0.884 | 0.874 | 0.894 | 0.932 | 0.882 | 0.983 | 0.929 | 0.849 | 1 |
| HERC6, IL1R2, FAM20A, RETN | | | | | | | | | |
| IFI27, OAS1, IFIT1, IFI44L, | 0.884 | 0.876 | 0.892 | 0.925 | 0.873 | 0.977 | 0.939 | 0.883 | 0.995 |
| SIGLEC1, HERC6, IL1R2, RETN | | | | | | | | | |
| IFI27, OAS1, SIGLEC1, HERC6, | 0.884 | 0.874 | 0.894 | 0.929 | 0.875 | 0.983 | 0.945 | 0.874 | 1 |
| SLC1A2, FAM20A, RETN, MMP8 | | | | | | | | | |
| RSAD2, IFI27, OAS1, IFIT1, | 0.884 | 0.874 | 0.894 | 0.929 | 0.878 | 0.981 | 0.920 | 0.837 | 1 |
| HERC6, SLC1A2, OLAH, FAM20A | | | | | | | | | |
| RSAD2, IFIT1, SIGLEC1, HERC6, | 0.884 | 0.875 | 0.893 | 0.924 | 0.871 | 0.978 | 0.962 | 0.906 | 1 |
| IL1R2, OLAH, FAM20A, MMP8 | | | | | | | | | |
| IFI27, OAS1, IFI44L, ISG15, IL1R2, | 0.884 | 0.875 | 0.893 | 0.924 | 0.867 | 0.981 | 0.928 | 0.856 | 1 |
| OLAH, FAM20A, RETN | | | | | | | | | |
| IFI27, OAS1, SIGLEC1, SLC1A2, | 0.884 | 0.874 | 0.894 | 0.930 | 0.877 | 0.982 | 0.941 | 0.876 | 1 |
| IL1R2, OLAH, FAM20A, RETN | | | | | | | | | |
| RSAD2, IFI27, OAS1, HERC6, | 0.884 | 0.875 | 0.893 | 0.924 | 0.868 | 0.979 | 0.945 | 0.886 | 1 |
| IL1R2, OLAH, RETN, MMP8 | | | | | | | | | |
| RSAD2, IFI27, OAS1, ISG15, | 0.884 | 0.875 | 0.893 | 0.930 | 0.880 | 0.981 | 0.927 | 0.844 | 1 |
| HERC6, IL1R2, FAM20A, RETN | | | | | | | | | |
| IFI27, OAS1, ISG15, SLC1A2, | 0.884 | 0.875 | 0.893 | 0.935 | 0.888 | 0.981 | 0.920 | 0.839 | 1.000 |
| IL1R2, OLAH, FAM20A, MMP8 | | | | | | | | | |
| IFI27, IFI44L, SIGLEC1, ISG15, | 0.884 | 0.876 | 0.892 | 0.926 | 0.877 | 0.975 | 0.940 | 0.886 | 0.994 |
| HERC6, IL1R2, OLAH, MMP8 | | | | | | | | | |
| RSAD2, IFI27, ISG15, HERC6, | 0.884 | 0.875 | 0.893 | 0.932 | 0.883 | 0.981 | 0.917 | 0.834 | 1 |
| SLC1A2, IL1R2, OLAH, FAM20A | | | | | | | | | |
| IFI27, OAS1, ISG15, HERC6, | 0.884 | 0.874 | 0.894 | 0.933 | 0.886 | 0.981 | 0.914 | 0.828 | 1 |
| SLC1A2, IL1R2, OLAH, FAM20A | | | | | | | | | |
| RSAD2, IFI27, OAS1, SIGLEC1, | 0.884 | 0.875 | 0.893 | 0.924 | 0.871 | 0.978 | 0.960 | 0.912 | 1 |
| HERC6, IL1R2, RETN, MMP8 | | | | | | | | | |
| RSAD2, IFI27, IFIT1, IFI44L, | 0.884 | 0.875 | 0.892 | 0.925 | 0.872 | 0.978 | 0.948 | 0.897 | 0.999 |
| SIGLEC1, HERC6, OLAH, RETN | | | | | | | | | |
| RSAD2, IFI27, OAS1, IFI44L, | 0.884 | 0.874 | 0.894 | 0.930 | 0.879 | 0.981 | 0.922 | 0.842 | 1 |
| HERC6, SLC1A2, OLAH, FAM20A | | | | | | | | | |
| IFI27, OAS1, IFI44L, SIGLEC1, | 0.884 | 0.875 | 0.893 | 0.928 | 0.877 | 0.978 | 0.951 | 0.903 | 0.999 |
| HERC6, IL1R2, RETN, MMP8 | | | | | | | | | |
| IFI27, OAS1, SIGLEC1, ISG15, | 0.884 | 0.875 | 0.893 | 0.923 | 0.867 | 0.978 | 0.946 | 0.890 | 1 |
| HERC6, IL1R2, OLAH, RETN | | | | | | | | | |
| IFI27, IFI44L, ISG15, SLC1A2, | 0.884 | 0.875 | 0.893 | 0.931 | 0.881 | 0.982 | 0.918 | 0.837 | 1.000 |
| IL1R2, OLAH, FAM20A, RETN | | | | | | | | | |
| IFI27, IFIT1, ISG15, HERC6, IL1R2, | 0.884 | 0.874 | 0.893 | 0.930 | 0.878 | 0.983 | 0.935 | 0.856 | 1 |
| FAM20A, RETN, MMP8 | | | | | | | | | |
| RSAD2, IFI27, OAS1, ISG15, | 0.884 | 0.875 | 0.893 | 0.924 | 0.869 | 0.980 | 0.946 | 0.888 | 1 |
| HERC6, OLAH, RETN, MMP8 | | | | | | | | | |
| RSAD2, IFI27, SIGLEC1, SLC1A2, | 0.884 | 0.874 | 0.894 | 0.929 | 0.876 | 0.982 | 0.946 | 0.882 | 1 |
| IL1R2, OLAH, FAM20A, RETN | | | | | | | | | |
| IFI27, OAS1, IFI44L, SIGLEC1, | 0.884 | 0.875 | 0.893 | 0.924 | 0.872 | 0.977 | 0.941 | 0.884 | 0.999 |
| ISG15, HERC6, OLAH, RETN | | | | | | | | | |
| IFI27, OAS1, IFI44L, SIGLEC1, | 0.884 | 0.875 | 0.892 | 0.924 | 0.872 | 0.977 | 0.942 | 0.885 | 1.000 |
| HERC6, IL1R2, OLAH, RETN | | | | | | | | | |
| IFI27, OAS1, IFIT1, IFI44L, IL1R2, | 0.884 | 0.875 | 0.893 | 0.924 | 0.867 | 0.982 | 0.930 | 0.858 | 1 |
| OLAH, FAM20A, RETN | | | | | | | | | |
| IFI27, IFIT1, IFI44L, SIGLEC1, | 0.884 | 0.875 | 0.893 | 0.934 | 0.886 | 0.981 | 0.943 | 0.874 | 1 |
| SLC1A2, IL1R2, FAM20A, RETN | | | | | | | | | |
| RSAD2, IFI27, OAS1, IFI44L, | 0.884 | 0.875 | 0.892 | 0.927 | 0.879 | 0.975 | 0.947 | 0.897 | 0.997 |
| SIGLEC1, HERC6, IL1R2, OLAH | | | | | | | | | |
| RSAD2, IFI27, SIGLEC1, HERC6, | 0.884 | 0.874 | 0.894 | 0.932 | 0.881 | 0.983 | 0.939 | 0.871 | 1 |
| SLC1A2, OLAH, FAM20A, RETN | | | | | | | | | |
| IFI27, OAS1, IFI44L, SIGLEC1, | 0.884 | 0.874 | 0.893 | 0.927 | 0.871 | 0.983 | 0.940 | 0.875 | 1 |
| SLC1A2, OLAH, FAM20A, RETN | | | | | | | | | |
| IFI27, OAS1, SIGLEC1, HERC6, | 0.884 | 0.874 | 0.894 | 0.935 | 0.889 | 0.980 | 0.940 | 0.869 | 1 |
| SLC1A2, IL1R2, FAM20A, RETN | | | | | | | | | |
| IFI27, OAS1, IFIT1, IFI44L, | 0.884 | 0.874 | 0.893 | 0.924 | 0.867 | 0.981 | 0.945 | 0.887 | 1 |
| HERC6, OLAH, RETN, MMP8 | | | | | | | | | |
| IFI27, IFIT1, SIGLEC1, HERC6, | 0.884 | 0.874 | 0.893 | 0.936 | 0.890 | 0.982 | 0.945 | 0.875 | 1 |
| SLC1A2, IL1R2, FAM20A, RETN | | | | | | | | | |
| IFI27, OAS1, IFIT1, ISG15, OLAH, | 0.884 | 0.874 | 0.894 | 0.921 | 0.861 | 0.981 | 0.940 | 0.874 | 1 |
| FAM20A, RETN, MMP8 | | | | | | | | | |
| RSAD2, IFI27, IFIT1, SIGLEC1, | 0.884 | 0.874 | 0.893 | 0.930 | 0.875 | 0.984 | 0.942 | 0.877 | 1 |
| SLC1A2, OLAH, FAM20A, RETN | | | | | | | | | |
| IFI27, OAS1, IFI44L, SIGLEC1, | 0.884 | 0.874 | 0.893 | 0.932 | 0.884 | 0.980 | 0.941 | 0.871 | 1 |
| ISG15, SLC1A2, IL1R2, FAM20A | | | | | | | | | |
| RSAD2, IFI27, OAS1, IFIT1, | 0.884 | 0.874 | 0.894 | 0.924 | 0.865 | 0.982 | 0.947 | 0.877 | 1 |
| SIGLEC1, FAM20A, RETN, MMP8 | | | | | | | | | |
| RSAD2, IFI27, IFI44L, SIGLEC1, | 0.884 | 0.876 | 0.892 | 0.926 | 0.878 | 0.975 | 0.943 | 0.892 | 0.995 |
| HERC6, IL1R2, OLAH, MMP8 | | | | | | | | | |
| RSAD2, IFI27, IFIT1, IFI44L, | 0.884 | 0.874 | 0.893 | 0.932 | 0.883 | 0.982 | 0.921 | 0.841 | 1.000 |
| HERC6, SLC1A2, OLAH, FAM20A | | | | | | | | | |
| IFI27, IFI44L, SIGLEC1, ISG15, | 0.884 | 0.875 | 0.892 | 0.925 | 0.872 | 0.978 | 0.946 | 0.893 | 0.998 |
| HERC6, IL1R2, OLAH, RETN | | | | | | | | | |
| IFI27, IFIT1, IFI44L, SIGLEC1, | 0.884 | 0.875 | 0.892 | 0.926 | 0.874 | 0.979 | 0.947 | 0.895 | 0.999 |
| HERC6, IL1R2, OLAH, RETN | | | | | | | | | |
| RSAD2, IFI27, OAS1, ISG15, | 0.884 | 0.874 | 0.893 | 0.928 | 0.875 | 0.980 | 0.918 | 0.837 | 1 |
| SLC1A2, OLAH, FAM20A, MMP8 | | | | | | | | | |
| IFI27, IFI44L, ISG15, HERC6, | 0.884 | 0.874 | 0.893 | 0.934 | 0.885 | 0.982 | 0.918 | 0.836 | 1 |
| SLC1A2, IL1R2, OLAH, FAM20A | | | | | | | | | |
| RSAD2, IFI27, OAS1, ISG15, | 0.884 | 0.874 | 0.894 | 0.923 | 0.864 | 0.982 | 0.937 | 0.869 | 1 |
| OLAH, FAM20A, RETN, MMP8 | | | | | | | | | |
| RSAD2, IFI27, OAS1, SIGLEC1, | 0.884 | 0.875 | 0.893 | 0.924 | 0.869 | 0.979 | 0.946 | 0.889 | 1 |
| HERC6, IL1R2, OLAH, RETN | | | | | | | | | |
| IFI27, OAS1, IFIT1, IFI44L, OLAH, | 0.884 | 0.873 | 0.894 | 0.923 | 0.863 | 0.983 | 0.941 | 0.876 | 1 |
| FAM20A, RETN, MMP8 | | | | | | | | | |
| IFI27, OAS1, SIGLEC1, HERC6, | 0.884 | 0.874 | 0.893 | 0.932 | 0.883 | 0.981 | 0.948 | 0.888 | 1 |
| SLC1A2, OLAH, RETN, MMP8 | | | | | | | | | |
| IFI27, OAS1, IFIT1, HERC6, | 0.884 | 0.874 | 0.893 | 0.932 | 0.883 | 0.981 | 0.921 | 0.840 | 1 |
| SLC1A2, IL1R2, OLAH, FAM20A | | | | | | | | | |
| RSAD2, IFI27, SIGLEC1, ISG15, | 0.884 | 0.874 | 0.893 | 0.935 | 0.888 | 0.981 | 0.945 | 0.874 | 1 |
| HERC6, SLC1A2, IL1R2, FAM20A | | | | | | | | | |
| RSAD2, IFI27, OAS1, IFIT1, | 0.884 | 0.874 | 0.893 | 0.926 | 0.873 | 0.978 | 0.935 | 0.857 | 1 |
| IFI44L, IL1R2, FAM20A, MMP8 | | | | | | | | | |
| RSAD2, IFI27, OAS1, IFIT1, ISG15, | 0.884 | 0.875 | 0.893 | 0.929 | 0.878 | 0.979 | 0.925 | 0.842 | 1 |
| HERC6, IL1R2, FAM20A | | | | | | | | | |
| IFI27, IFI44L, SIGLEC1, ISG15, | 0.884 | 0.874 | 0.893 | 0.932 | 0.885 | 0.980 | 0.945 | 0.874 | 1 |
| SLC1A2, IL1R2, FAM20A, RETN | | | | | | | | | |
| IFI27, SIGLEC1, HERC6, SLC1A2, | 0.884 | 0.874 | 0.893 | 0.935 | 0.887 | 0.983 | 0.955 | 0.888 | 1 |
| IL1R2, FAM20A, RETN, MMP8 | | | | | | | | | |
| RSAD2, IFI27, OAS1, IFI44L, | 0.884 | 0.873 | 0.894 | 0.931 | 0.879 | 0.984 | 0.926 | 0.850 | 1 |
| SLC1A2, OLAH, FAM20A, MMP8 | | | | | | | | | |
| RSAD2, IFI27, IFI44L, ISG15, | 0.884 | 0.873 | 0.894 | 0.934 | 0.884 | 0.983 | 0.918 | 0.837 | 1.000 |
| HERC6, SLC1A2, OLAH, FAM20A | | | | | | | | | |
| IFI27, OAS1, IFIT1, IFI44L, IL1R2, | 0.884 | 0.874 | 0.893 | 0.929 | 0.876 | 0.982 | 0.935 | 0.858 | 1 |
| FAM20A, RETN, MMP8 | | | | | | | | | |
| IFI27, IFIT1, SIGLEC1, ISG15, | 0.884 | 0.874 | 0.893 | 0.933 | 0.886 | 0.980 | 0.943 | 0.874 | 1 |
| SLC1A2, IL1R2, FAM20A, RETN | | | | | | | | | |
| IFI27, OAS1, IFIT1, SIGLEC1, | 0.883 | 0.874 | 0.893 | 0.924 | 0.867 | 0.980 | 0.939 | 0.868 | 1 |
| ISG15, HERC6, FAM20A, MMP8 | | | | | | | | | |
| RSAD2, IFI27, OAS1, ISG15, | 0.883 | 0.874 | 0.893 | 0.924 | 0.868 | 0.980 | 0.925 | 0.850 | 1.000 |
| IL1R2, OLAH, FAM20A, RETN | | | | | | | | | |
| IFI27, OAS1, IFIT1, SIGLEC1, | 0.883 | 0.875 | 0.892 | 0.924 | 0.875 | 0.974 | 0.942 | 0.890 | 0.995 |
| HERC6, IL1R2, OLAH, MMP8 | | | | | | | | | |
| IFI27, ISG15, SLC1A2, IL1R2, | 0.883 | 0.874 | 0.893 | 0.933 | 0.883 | 0.982 | 0.921 | 0.840 | 1 |
| OLAH, FAM20A, RETN, MMP8 | | | | | | | | | |
| RSAD2, IFI27, IFIT1, ISG15, | 0.883 | 0.874 | 0.892 | 0.929 | 0.878 | 0.979 | 0.934 | 0.854 | 1 |
| HERC6, IL1R2, FAM20A, MMP8 | | | | | | | | | |
| IFI27, OAS1, IFI44L, HERC6, | 0.883 | 0.873 | 0.894 | 0.931 | 0.881 | 0.982 | 0.921 | 0.839 | 1 |
| SLC1A2, IL1R2, OLAH, FAM20A | | | | | | | | | |
| OAS1, IFIT1, SIGLEC1, HERC6, | 0.883 | 0.874 | 0.892 | 0.923 | 0.868 | 0.979 | 0.962 | 0.905 | 1 |
| IL1R2, FAM20A, RETN, MMP8 | | | | | | | | | |
| RSAD2, IFIT1, IFI44L, SIGLEC1, | 0.883 | 0.875 | 0.892 | 0.924 | 0.870 | 0.978 | 0.962 | 0.906 | 1 |
| HERC6, IL1R2, FAM20A, MMP8 | | | | | | | | | |
| OAS1, IFIT1, SIGLEC1, ISG15, | 0.883 | 0.874 | 0.892 | 0.923 | 0.868 | 0.978 | 0.963 | 0.907 | 1 |
| HERC6, IL1R2, FAM20A, MMP8 | | | | | | | | | |
| IFI27, SIGLEC1, ISG15, HERC6, | 0.883 | 0.873 | 0.893 | 0.931 | 0.878 | 0.983 | 0.939 | 0.872 | 1 |
| SLC1A2, OLAH, FAM20A, RETN | | | | | | | | | |
| IFI27, OAS1, IFIT1, SIGLEC1, | 0.883 | 0.874 | 0.893 | 0.933 | 0.886 | 0.981 | 0.943 | 0.875 | 1 |
| SLC1A2, IL1R2, FAM20A, RETN | | | | | | | | | |
| RSAD2, IFI27, OAS1, IFIT1, | 0.883 | 0.875 | 0.892 | 0.925 | 0.872 | 0.978 | 0.951 | 0.897 | 1 |
| SIGLEC1, HERC6, IL1R2, RETN | | | | | | | | | |
| RSAD2, IFI27, OAS1, IFIT1, | 0.883 | 0.874 | 0.893 | 0.924 | 0.867 | 0.980 | 0.926 | 0.850 | 1 |
| SLC1A2, OLAH, FAM20A, MMP8 | | | | | | | | | |
| RSAD2, IFI27, IFIT1, IFI44L, | 0.883 | 0.874 | 0.892 | 0.923 | 0.864 | 0.981 | 0.940 | 0.869 | 1 |
| SIGLEC1, HERC6, FAM20A, MMP8 | | | | | | | | | |
| IFI27, OAS1, ISG15, HERC6, | 0.883 | 0.874 | 0.893 | 0.932 | 0.883 | 0.982 | 0.915 | 0.829 | 1 |
| SLC1A2, OLAH, FAM20A, RETN | | | | | | | | | |
| IFI27, OAS1, IFIT1, IFI44L, | 0.883 | 0.875 | 0.892 | 0.926 | 0.875 | 0.977 | 0.943 | 0.890 | 0.997 |
| SIGLEC1, HERC6, OLAH, MMP8 | | | | | | | | | |
| IFI27, SIGLEC1, HERC6, SLC1A2, | 0.883 | 0.873 | 0.894 | 0.927 | 0.871 | 0.983 | 0.948 | 0.881 | 1 |
| OLAH, FAM20A, RETN, MMP8 | | | | | | | | | |
| IFIT1, IFI44L, SIGLEC1, HERC6, | 0.883 | 0.874 | 0.892 | 0.925 | 0.870 | 0.980 | 0.964 | 0.909 | 1 |
| IL1R2, FAM20A, RETN, MMP8 | | | | | | | | | |
| IFI27, OAS1, IFIT1, SIGLEC1, | 0.883 | 0.874 | 0.893 | 0.933 | 0.886 | 0.980 | 0.943 | 0.875 | 1 |
| ISG15, SLC1A2, IL1R2, FAM20A | | | | | | | | | |
| IFI27, IFIT1, IFI44L, SIGLEC1, | 0.883 | 0.875 | 0.891 | 0.927 | 0.878 | 0.976 | 0.942 | 0.889 | 0.996 |
| ISG15, HERC6, IL1R2, OLAH | | | | | | | | | |
| RSAD2, IFI27, IFIT1, IFI44L, | 0.883 | 0.874 | 0.892 | 0.920 | 0.861 | 0.980 | 0.943 | 0.874 | 1 |
| SIGLEC1, ISG15, FAM20A, RETN | | | | | | | | | |
| IFI27, OAS1, IFI44L, ISG15, OLAH, | 0.883 | 0.873 | 0.894 | 0.923 | 0.864 | 0.983 | 0.941 | 0.875 | 1 |
| FAM20A, RETN, MMP8 | | | | | | | | | |
| IFI27, OAS1, IFIT1, SIGLEC1, | 0.883 | 0.875 | 0.892 | 0.924 | 0.872 | 0.976 | 0.947 | 0.891 | 1 |
| ISG15, HERC6, IL1R2, RETN | | | | | | | | | |
| RSAD2, IFI27, OAS1, IFI44L, | 0.883 | 0.875 | 0.891 | 0.924 | 0.872 | 0.977 | 0.954 | 0.904 | 1 |
| SIGLEC1, HERC6, IL1R2, RETN | | | | | | | | | |
| RSAD2, IFI27, IFI44L, SIGLEC1, | 0.883 | 0.873 | 0.893 | 0.932 | 0.883 | 0.980 | 0.948 | 0.879 | 1 |
| HERC6, SLC1A2, IL1R2, FAM20A | | | | | | | | | |
| IFI27, OAS1, IFI44L, SIGLEC1, | 0.883 | 0.875 | 0.891 | 0.927 | 0.877 | 0.977 | 0.939 | 0.883 | 0.995 |
| ISG15, HERC6, IL1R2, RETN | | | | | | | | | |
| RSAD2, IFI27, OAS1, IFIT1, | 0.883 | 0.874 | 0.892 | 0.923 | 0.866 | 0.980 | 0.938 | 0.867 | 1 |
| SIGLEC1, HERC6, FAM20A, MMP8 | | | | | | | | | |
| IFI27, ISG15, HERC6, SLC1A2, | 0.883 | 0.873 | 0.893 | 0.931 | 0.880 | 0.982 | 0.922 | 0.839 | 1 |
| OLAH, FAM20A, RETN, MMP8 | | | | | | | | | |
| IFI27, OAS1, SIGLEC1, ISG15, | 0.883 | 0.874 | 0.892 | 0.926 | 0.873 | 0.979 | 0.957 | 0.907 | 1 |
| HERC6, IL1R2, RETN, MMP8 | | | | | | | | | |
| RSAD2, IFI27, IFI44L, ISG15, | 0.883 | 0.873 | 0.893 | 0.932 | 0.881 | 0.982 | 0.929 | 0.849 | 1 |
| HERC6, IL1R2, FAM20A, RETN | | | | | | | | | |
| IFI27, IFIT1, IFI44L, HERC6, | 0.883 | 0.874 | 0.892 | 0.931 | 0.882 | 0.981 | 0.925 | 0.845 | 1 |
| SLC1A2, IL1R2, OLAH, FAM20A | | | | | | | | | |
| IFI27, OAS1, SIGLEC1, ISG15, | 0.883 | 0.874 | 0.892 | 0.924 | 0.875 | 0.973 | 0.943 | 0.891 | 0.996 |
| HERC6, IL1R2, OLAH, MMP8 | | | | | | | | | |
| RSAD2, IFI27, IFIT1, IFI44L, | 0.883 | 0.874 | 0.892 | 0.924 | 0.867 | 0.981 | 0.932 | 0.861 | 1 |
| ISG15, IL1R2, OLAH, FAM20A | | | | | | | | | |
| IFI27, OAS1, IFIT1, HERC6, | 0.883 | 0.873 | 0.893 | 0.932 | 0.882 | 0.982 | 0.916 | 0.830 | 1 |
| SLC1A2, OLAH, FAM20A, RETN | | | | | | | | | |
| RSAD2, IFI27, OAS1, IFIT1, | 0.883 | 0.874 | 0.892 | 0.921 | 0.861 | 0.981 | 0.932 | 0.863 | 1 |
| IFI44L, ISG15, OLAH, FAM20A | | | | | | | | | |
| RSAD2, IFI27, IFIT1, SIGLEC1, | 0.883 | 0.873 | 0.893 | 0.923 | 0.863 | 0.982 | 0.946 | 0.874 | 1 |
| ISG15, FAM20A, RETN, MMP8 | | | | | | | | | |
| RSAD2, IFI27, IFI44L, SIGLEC1, | 0.883 | 0.874 | 0.892 | 0.924 | 0.872 | 0.977 | 0.947 | 0.895 | 0.999 |
| HERC6, IL1R2, OLAH, RETN | | | | | | | | | |
| RSAD2, IFI27, OAS1, IFI44L, | 0.883 | 0.874 | 0.892 | 0.924 | 0.867 | 0.980 | 0.941 | 0.882 | 1 |
| HERC6, OLAH, RETN, MMP8 | | | | | | | | | |
| IFIT1, SIGLEC1, HERC6, IL1R2, | 0.883 | 0.874 | 0.892 | 0.925 | 0.870 | 0.980 | 0.961 | 0.904 | 1 |
| OLAH, FAM20A, RETN, MMP8 | | | | | | | | | |
| IFI27, OAS1, IFIT1, IFI44L, | 0.883 | 0.875 | 0.891 | 0.926 | 0.877 | 0.975 | 0.946 | 0.895 | 0.996 |
| SIGLEC1, HERC6, IL1R2, OLAH | | | | | | | | | |
| IFI27, OAS1, IFI44L, SLC1A2, | 0.883 | 0.873 | 0.893 | 0.934 | 0.884 | 0.983 | 0.927 | 0.851 | 1 |
| IL1R2, OLAH, FAM20A, MMP8 | | | | | | | | | |
| IFI27, IFIT1, SIGLEC1, ISG15, | 0.883 | 0.874 | 0.892 | 0.923 | 0.869 | 0.978 | 0.947 | 0.894 | 0.999 |
| HERC6, IL1R2, OLAH, RETN | | | | | | | | | |
| IFI27, OAS1, IFI44L, SIGLEC1, | 0.883 | 0.874 | 0.891 | 0.926 | 0.877 | 0.975 | 0.941 | 0.888 | 0.995 |
| ISG15, HERC6, IL1R2, OLAH | | | | | | | | | |
| RSAD2, IFI27, IFIT1, ISG15, | 0.883 | 0.874 | 0.892 | 0.932 | 0.881 | 0.983 | 0.929 | 0.849 | 1 |
| HERC6, IL1R2, FAM20A, RETN | | | | | | | | | |
| RSAD2, IFI27, OAS1, SIGLEC1, | 0.883 | 0.874 | 0.892 | 0.924 | 0.874 | 0.973 | 0.943 | 0.891 | 0.996 |
| HERC6, IL1R2, OLAH, MMP8 | | | | | | | | | |
| IFI27, OAS1, IFI44L, HERC6, | 0.883 | 0.874 | 0.892 | 0.924 | 0.869 | 0.979 | 0.940 | 0.882 | 0.999 |
| IL1R2, OLAH, RETN, MMP8 | | | | | | | | | |
| IFI27, OAS1, IFI44L, ISG15, | 0.883 | 0.874 | 0.892 | 0.925 | 0.869 | 0.980 | 0.942 | 0.883 | 1 |
| HERC6, OLAH, RETN, MMP8 | | | | | | | | | |
| RSAD2, IFI27, OAS1, IFIT1, | 0.883 | 0.873 | 0.892 | 0.918 | 0.858 | 0.979 | 0.945 | 0.876 | 1 |
| IFI44L, SIGLEC1, FAM20A, RETN | | | | | | | | | |
| IFI27, OAS1, IFI44L, SIGLEC1, | 0.883 | 0.874 | 0.892 | 0.920 | 0.863 | 0.978 | 0.945 | 0.884 | 1 |
| ISG15, HERC6, RETN, MMP8 | | | | | | | | | |
| RSAD2, IFI27, OAS1, IFIT1, | 0.883 | 0.874 | 0.892 | 0.923 | 0.866 | 0.980 | 0.939 | 0.868 | 1 |
| SIGLEC1, ISG15, HERC6, FAM20A | | | | | | | | | |
| RSAD2, IFI27, IFIT1, HERC6, | 0.883 | 0.874 | 0.892 | 0.924 | 0.869 | 0.980 | 0.945 | 0.889 | 1 |
| IL1R2, OLAH, RETN, MMP8 | | | | | | | | | |
| RSAD2, IFI27, OAS1, IFI44L, | 0.883 | 0.874 | 0.891 | 0.920 | 0.863 | 0.978 | 0.942 | 0.878 | 1 |
| SIGLEC1, ISG15, HERC6, RETN | | | | | | | | | |
| RSAD2, IFI27, OAS1, IFI44L, | 0.883 | 0.874 | 0.891 | 0.926 | 0.875 | 0.977 | 0.945 | 0.892 | 0.997 |
| SIGLEC1, HERC6, OLAH, MMP8 | | | | | | | | | |
| RSAD2, IFI27, IFI44L, HERC6, | 0.883 | 0.873 | 0.893 | 0.931 | 0.881 | 0.981 | 0.925 | 0.847 | 1 |
| SLC1A2, IL1R2, OLAH, FAM20A | | | | | | | | | |
| RSAD2, IFI27, OAS1, IFI44L, | 0.883 | 0.873 | 0.893 | 0.925 | 0.869 | 0.981 | 0.934 | 0.856 | 1 |
| ISG15, HERC6, FAM20A, RETN | | | | | | | | | |
| IFI27, IFIT1, SLC1A2, IL1R2, | 0.883 | 0.873 | 0.892 | 0.932 | 0.881 | 0.982 | 0.923 | 0.843 | 1 |
| OLAH, FAM20A, RETN, MMP8 | | | | | | | | | |
| IFI27, IFI44L, ISG15, HERC6, | 0.883 | 0.874 | 0.892 | 0.923 | 0.867 | 0.979 | 0.945 | 0.890 | 0.999 |
| IL1R2, OLAH, RETN, MMP8 | | | | | | | | | |
| IFI27, OAS1, IFIT1, IFI44L, ISG15, | 0.883 | 0.873 | 0.892 | 0.920 | 0.859 | 0.980 | 0.934 | 0.864 | 1 |
| OLAH, FAM20A, RETN | | | | | | | | | |
| RSAD2, IFI27, SIGLEC1, ISG15, | 0.883 | 0.873 | 0.892 | 0.924 | 0.868 | 0.980 | 0.954 | 0.905 | 1 |
| HERC6, OLAH, RETN, MMP8 | | | | | | | | | |
| RSAD2, IFI27, OAS1, IFIT1, | 0.883 | 0.874 | 0.892 | 0.919 | 0.860 | 0.978 | 0.947 | 0.889 | 1 |
| SIGLEC1, HERC6, RETN, MMP8 | | | | | | | | | |
| RSAD2, IFI27, OAS1, SIGLEC1, | 0.883 | 0.874 | 0.892 | 0.924 | 0.869 | 0.979 | 0.946 | 0.889 | 1 |
| ISG15, HERC6, OLAH, RETN | | | | | | | | | |
| RSAD2, IFI27, OAS1, IFIT1, | 0.883 | 0.874 | 0.892 | 0.923 | 0.867 | 0.979 | 0.940 | 0.880 | 1 |
| HERC6, IL1R2, OLAH, RETN | | | | | | | | | |
| RSAD2, IFI27, IFI44L, SIGLEC1, | 0.883 | 0.873 | 0.893 | 0.929 | 0.876 | 0.982 | 0.946 | 0.881 | 1 |
| SLC1A2, IL1R2, OLAH, FAM20A | | | | | | | | | |
| RSAD2, IFI27, IFIT1, IFI44L, | 0.883 | 0.873 | 0.892 | 0.925 | 0.870 | 0.980 | 0.932 | 0.853 | 1 |
| ISG15, HERC6, FAM20A, RETN | | | | | | | | | |
| IFI27, OAS1, IFIT1, IFI44L, ISG15, | 0.883 | 0.873 | 0.892 | 0.930 | 0.877 | 0.983 | 0.920 | 0.840 | 0.999 |
| SLC1A2, OLAH, FAM20A | | | | | | | | | |
| IFI27, OAS1, IFIT1, IFI44L, | 0.883 | 0.874 | 0.891 | 0.920 | 0.863 | 0.977 | 0.943 | 0.884 | 1 |
| SIGLEC1, HERC6, RETN, MMP8 | | | | | | | | | |
| IFI27, IFIT1, HERC6, SLC1A2, | 0.883 | 0.873 | 0.893 | 0.931 | 0.880 | 0.982 | 0.924 | 0.842 | 1 |
| OLAH, FAM20A, RETN, MMP8 | | | | | | | | | |
| IFI27, OAS1, IFI44L, SIGLEC1, | 0.883 | 0.873 | 0.892 | 0.932 | 0.883 | 0.981 | 0.941 | 0.870 | 1 |
| SLC1A2, IL1R2, FAM20A, RETN | | | | | | | | | |
| RSAD2, IFIT1, SIGLEC1, ISG15, | 0.883 | 0.874 | 0.891 | 0.922 | 0.867 | 0.977 | 0.963 | 0.907 | 1 |
| HERC6, IL1R2, FAM20A, MMP8 | | | | | | | | | |
| IFIT1, SIGLEC1, ISG15, HERC6, | 0.883 | 0.874 | 0.891 | 0.923 | 0.867 | 0.979 | 0.961 | 0.901 | 1 |
| IL1R2, FAM20A, RETN, MMP8 | | | | | | | | | |
| RSAD2, IFI27, OAS1, IFIT1, | 0.883 | 0.873 | 0.892 | 0.920 | 0.861 | 0.980 | 0.943 | 0.875 | 1 |
| SIGLEC1, ISG15, FAM20A, RETN | | | | | | | | | |
| IFI27, IFIT1, IFI44L, ISG15, IL1R2, | 0.883 | 0.873 | 0.892 | 0.929 | 0.876 | 0.982 | 0.937 | 0.860 | 1 |
| FAM20A, RETN, MMP8 | | | | | | | | | |
| RSAD2, IFI27, IFIT1, ISG15, | 0.883 | 0.873 | 0.893 | 0.931 | 0.880 | 0.981 | 0.917 | 0.835 | 0.999 |
| HERC6, SLC1A2, OLAH, FAM20A | | | | | | | | | |
| RSAD2, IFI27, IFIT1, IFI44L, | 0.883 | 0.872 | 0.893 | 0.922 | 0.862 | 0.982 | 0.938 | 0.871 | 1 |
| OLAH, FAM20A, RETN, MMP8 | | | | | | | | | |
| RSAD2, IFI27, HERC6, SLC1A2, | 0.882 | 0.873 | 0.892 | 0.931 | 0.881 | 0.981 | 0.920 | 0.835 | 1 |
| IL1R2, OLAH, FAM20A, RETN | | | | | | | | | |
| RSAD2, IFI27, IFIT1, HERC6, | 0.882 | 0.873 | 0.892 | 0.931 | 0.881 | 0.980 | 0.923 | 0.842 | 1 |
| SLC1A2, IL1R2, OLAH, FAM20A | | | | | | | | | |
| RSAD2, IFI27, ISG15, HERC6, | 0.882 | 0.873 | 0.892 | 0.930 | 0.878 | 0.981 | 0.914 | 0.829 | 1.000 |
| SLC1A2, OLAH, FAM20A, RETN | | | | | | | | | |
| RSAD2, IFI27, OAS1, IFIT1, | 0.882 | 0.873 | 0.892 | 0.924 | 0.866 | 0.981 | 0.933 | 0.853 | 1 |
| IFI44L, HERC6, FAM20A, RETN | | | | | | | | | |
| RSAD2, IFI27, IFIT1, IFI44L, | 0.882 | 0.873 | 0.891 | 0.935 | 0.888 | 0.982 | 0.943 | 0.874 | 1 |
| SIGLEC1, SLC1A2, IL1R2, FAM20A | | | | | | | | | |
| RSAD2, IFI27, IFIT1, IFI44L, | 0.882 | 0.873 | 0.891 | 0.923 | 0.867 | 0.980 | 0.949 | 0.895 | 1 |
| HERC6, OLAH, RETN, MMP8 | | | | | | | | | |
| RSAD2, IFI27, IFIT1, ISG15, | 0.882 | 0.872 | 0.893 | 0.921 | 0.860 | 0.981 | 0.940 | 0.875 | 1 |
| OLAH, FAM20A, RETN, MMP8 | | | | | | | | | |
| RSAD2, IFI27, OAS1, IFIT1, | 0.882 | 0.874 | 0.891 | 0.921 | 0.864 | 0.978 | 0.940 | 0.881 | 1.000 |
| IFI44L, HERC6, OLAH, RETN | | | | | | | | | |
| RSAD2, IFI27, IFIT1, IFI44L, | 0.882 | 0.872 | 0.892 | 0.923 | 0.864 | 0.982 | 0.947 | 0.877 | 1 |
| SIGLEC1, FAM20A, RETN, MMP8 | | | | | | | | | |
| IFI27, OAS1, IFIT1, SIGLEC1, | 0.882 | 0.874 | 0.891 | 0.923 | 0.871 | 0.975 | 0.936 | 0.878 | 0.994 |
| ISG15, HERC6, OLAH, MMP8 | | | | | | | | | |
| RSAD2, IFI27, IFI44L, ISG15, | 0.882 | 0.873 | 0.892 | 0.923 | 0.866 | 0.980 | 0.928 | 0.856 | 1 |
| IL1R2, OLAH, FAM20A, RETN | | | | | | | | | |
| IFI27, IFIT1, IFI44L, ISG15, | 0.882 | 0.873 | 0.891 | 0.931 | 0.880 | 0.982 | 0.917 | 0.836 | 0.999 |
| SLC1A2, IL1R2, OLAH, FAM20A | | | | | | | | | |
| IFI27, OAS1, IFIT1, ISG15, HERC6, | 0.882 | 0.874 | 0.891 | 0.922 | 0.866 | 0.977 | 0.939 | 0.878 | 1.000 |
| IL1R2, OLAH, RETN | | | | | | | | | |
| RSAD2, IFI27, OAS1, ISG15, | 0.882 | 0.873 | 0.891 | 0.923 | 0.867 | 0.979 | 0.943 | 0.885 | 1 |
| HERC6, IL1R2, OLAH, RETN | | | | | | | | | |
| RSAD2, IFI27, OAS1, HERC6, | 0.882 | 0.873 | 0.892 | 0.932 | 0.883 | 0.982 | 0.917 | 0.831 | 1 |
| SLC1A2, OLAH, FAM20A, RETN | | | | | | | | | |
| RSAD2, IFI27, SLC1A2, IL1R2, | 0.882 | 0.873 | 0.892 | 0.933 | 0.883 | 0.982 | 0.922 | 0.840 | 1 |
| OLAH, FAM20A, RETN, MMP8 | | | | | | | | | |
| RSAD2, IFI27, IFIT1, ISG15, IL1R2, | 0.882 | 0.873 | 0.891 | 0.924 | 0.867 | 0.981 | 0.932 | 0.861 | 1 |
| OLAH, FAM20A, RETN | | | | | | | | | |
| IFI27, OAS1, IFIT1, SIGLEC1, | 0.882 | 0.874 | 0.891 | 0.924 | 0.874 | 0.974 | 0.942 | 0.889 | 0.995 |
| ISG15, HERC6, IL1R2, OLAH | | | | | | | | | |
| RSAD2, IFI27, OAS1, SIGLEC1, | 0.882 | 0.872 | 0.893 | 0.925 | 0.866 | 0.983 | 0.945 | 0.872 | 1 |
| ISG15, FAM20A, RETN, MMP8 | | | | | | | | | |
| IFI27, IFIT1, ISG15, HERC6, | 0.882 | 0.873 | 0.892 | 0.932 | 0.883 | 0.981 | 0.920 | 0.838 | 1 |
| SLC1A2, IL1R2, OLAH, FAM20A | | | | | | | | | |
| RSAD2, IFI27, IFI44L, HERC6, | 0.882 | 0.873 | 0.891 | 0.924 | 0.869 | 0.980 | 0.945 | 0.889 | 1 |
| IL1R2, OLAH, RETN, MMP8 | | | | | | | | | |
| RSAD2, IFI27, OAS1, IFI44L, | 0.882 | 0.872 | 0.893 | 0.923 | 0.864 | 0.982 | 0.938 | 0.870 | 1 |
| OLAH, FAM20A, RETN, MMP8 | | | | | | | | | |
| RSAD2, IFI27, IFIT1, IFI44L, | 0.882 | 0.874 | 0.891 | 0.928 | 0.879 | 0.976 | 0.943 | 0.892 | 0.995 |
| SIGLEC1, HERC6, IL1R2, OLAH | | | | | | | | | |
| RSAD2, IFI27, OAS1, SIGLEC1, | 0.882 | 0.873 | 0.891 | 0.924 | 0.874 | 0.974 | 0.940 | 0.885 | 0.996 |
| ISG15, HERC6, IL1R2, OLAH | | | | | | | | | |
| RSAD2, IFI27, IFIT1, SIGLEC1, | 0.882 | 0.873 | 0.891 | 0.934 | 0.888 | 0.980 | 0.945 | 0.875 | 1 |
| ISG15, SLC1A2, IL1R2, FAM20A | | | | | | | | | |
| RSAD2, IFI27, OAS1, SIGLEC1, | 0.882 | 0.873 | 0.892 | 0.927 | 0.871 | 0.983 | 0.939 | 0.872 | 1 |
| SLC1A2, OLAH, FAM20A, RETN | | | | | | | | | |
| IFI27, HERC6, SLC1A2, IL1R2, | 0.882 | 0.872 | 0.892 | 0.933 | 0.883 | 0.982 | 0.928 | 0.851 | 1 |
| OLAH, FAM20A, RETN, MMP8 | | | | | | | | | |
| IFI27, IFIT1, IFI44L, ISG15, | 0.882 | 0.873 | 0.891 | 0.922 | 0.864 | 0.979 | 0.947 | 0.894 | 1.000 |
| HERC6, OLAH, RETN, MMP8 | | | | | | | | | |
| RSAD2, IFI27, OAS1, IFIT1, IL1R2, | 0.882 | 0.873 | 0.892 | 0.930 | 0.877 | 0.982 | 0.934 | 0.856 | 1 |
| FAM20A, RETN, MMP8 | | | | | | | | | |
| IFI27, IFIT1, IFI44L, SIGLEC1, | 0.882 | 0.873 | 0.891 | 0.925 | 0.873 | 0.977 | 0.945 | 0.892 | 0.997 |
| ISG15, HERC6, OLAH, MMP8 | | | | | | | | | |
| IFI27, IFIT1, IFI44L, SIGLEC1, | 0.882 | 0.874 | 0.890 | 0.926 | 0.877 | 0.975 | 0.945 | 0.894 | 0.995 |
| HERC6, IL1R2, OLAH, MMP8 | | | | | | | | | |
| RSAD2, IFI27, IFI44L, SIGLEC1, | 0.882 | 0.874 | 0.891 | 0.924 | 0.870 | 0.978 | 0.946 | 0.893 | 0.998 |
| ISG15, HERC6, OLAH, RETN | | | | | | | | | |
| RSAD2, IFI27, IFIT1, SIGLEC1, | 0.882 | 0.873 | 0.891 | 0.922 | 0.864 | 0.980 | 0.940 | 0.869 | 1 |
| ISG15, HERC6, FAM20A, MMP8 | | | | | | | | | |
| RSAD2, IFI27, IFI44L, ISG15, | 0.882 | 0.872 | 0.893 | 0.923 | 0.864 | 0.983 | 0.939 | 0.872 | 1 |
| OLAH, FAM20A, RETN, MMP8 | | | | | | | | | |
| RSAD2, IFI27, ISG15, HERC6, | 0.882 | 0.873 | 0.891 | 0.925 | 0.870 | 0.980 | 0.943 | 0.887 | 1 |
| IL1R2, OLAH, RETN, MMP8 | | | | | | | | | |
| RSAD2, IFI27, IFIT1, SIGLEC1, | 0.882 | 0.873 | 0.891 | 0.926 | 0.873 | 0.978 | 0.942 | 0.887 | 0.998 |
| ISG15, HERC6, OLAH, RETN | | | | | | | | | |
| RSAD2, IFI27, OAS1, IFIT1, ISG15, | 0.882 | 0.872 | 0.892 | 0.923 | 0.867 | 0.980 | 0.935 | 0.857 | 1 |
| HERC6, FAM20A, RETN | | | | | | | | | |
| RSAD2, IFI27, OAS1, IFIT1, IL1R2, | 0.882 | 0.873 | 0.891 | 0.924 | 0.868 | 0.981 | 0.933 | 0.863 | 1 |
| OLAH, FAM20A, RETN | | | | | | | | | |
| RSAD2, OAS1, IFIT1, SIGLEC1, | 0.882 | 0.873 | 0.891 | 0.919 | 0.862 | 0.977 | 0.964 | 0.910 | 1 |
| IL1R2, OLAH, FAM20A, MMP8 | | | | | | | | | |
| RSAD2, IFI27, OAS1, IFIT1, ISG15, | 0.882 | 0.873 | 0.891 | 0.923 | 0.868 | 0.978 | 0.941 | 0.882 | 1 |
| HERC6, OLAH, RETN | | | | | | | | | |
| RSAD2, IFI27, IFIT1, SIGLEC1, | 0.882 | 0.873 | 0.891 | 0.926 | 0.873 | 0.978 | 0.942 | 0.887 | 0.998 |
| HERC6, IL1R2, OLAH, RETN | | | | | | | | | |
| IFI27, OAS1, IFI44L, HERC6, | 0.882 | 0.872 | 0.892 | 0.932 | 0.882 | 0.982 | 0.920 | 0.837 | 1 |
| SLC1A2, OLAH, FAM20A, RETN | | | | | | | | | |
| IFI27, OAS1, IFI44L, ISG15, | 0.882 | 0.872 | 0.892 | 0.931 | 0.880 | 0.983 | 0.916 | 0.833 | 1.000 |
| SLC1A2, IL1R2, OLAH, FAM20A | | | | | | | | | |
| IFI27, OAS1, IFI44L, ISG15, | 0.882 | 0.871 | 0.892 | 0.927 | 0.869 | 0.984 | 0.937 | 0.859 | 1 |
| HERC6, FAM20A, RETN, MMP8 | | | | | | | | | |
| RSAD2, IFI27, OAS1, ISG15, | 0.882 | 0.872 | 0.891 | 0.932 | 0.883 | 0.981 | 0.916 | 0.832 | 1 |
| SLC1A2, IL1R2, OLAH, FAM20A | | | | | | | | | |
| RSAD2, IFI27, IFIT1, SIGLEC1, | 0.882 | 0.873 | 0.891 | 0.929 | 0.878 | 0.981 | 0.938 | 0.865 | 1 |
| HERC6, SLC1A2, FAM20A, RETN | | | | | | | | | |
| RSAD2, IFI27, OAS1, IFIT1, | 0.882 | 0.873 | 0.891 | 0.923 | 0.872 | 0.974 | 0.938 | 0.881 | 0.995 |
| SIGLEC1, HERC6, OLAH, MMP8 | | | | | | | | | |
| IFI27, IFIT1, IFI44L, ISG15, | 0.882 | 0.873 | 0.891 | 0.928 | 0.874 | 0.982 | 0.916 | 0.834 | 0.999 |
| SLC1A2, OLAH, FAM20A, RETN | | | | | | | | | |
| RSAD2, IFI27, IFI44L, ISG15, | 0.882 | 0.872 | 0.892 | 0.931 | 0.880 | 0.982 | 0.918 | 0.837 | 1.000 |
| SLC1A2, IL1R2, OLAH, FAM20A | | | | | | | | | |
| IFI27, IFIT1, IFI44L, SIGLEC1, | 0.882 | 0.873 | 0.890 | 0.924 | 0.872 | 0.977 | 0.953 | 0.908 | 0.999 |
| HERC6, IL1R2, RETN, MMP8 | | | | | | | | | |
| RSAD2, IFI27, IFI44L, SIGLEC1, | 0.882 | 0.873 | 0.890 | 0.926 | 0.876 | 0.976 | 0.942 | 0.889 | 0.996 |
| ISG15, HERC6, IL1R2, OLAH | | | | | | | | | |
| RSAD2, IFI27, IFIT1, IFI44L, | 0.882 | 0.873 | 0.891 | 0.928 | 0.874 | 0.982 | 0.916 | 0.834 | 0.999 |
| ISG15, SLC1A2, OLAH, FAM20A | | | | | | | | | |
| IFI27, OAS1, IFIT1, IFI44L, | 0.882 | 0.873 | 0.890 | 0.927 | 0.876 | 0.978 | 0.942 | 0.888 | 0.997 |
| SIGLEC1, ISG15, HERC6, OLAH | | | | | | | | | |
| IFI27, OAS1, IFIT1, ISG15, IL1R2, | 0.882 | 0.872 | 0.892 | 0.930 | 0.877 | 0.983 | 0.934 | 0.856 | 1 |
| FAM20A, RETN, MMP8 | | | | | | | | | |
| RSAD2, IFI27, SIGLEC1, HERC6, | 0.882 | 0.872 | 0.892 | 0.934 | 0.886 | 0.982 | 0.946 | 0.876 | 1 |
| SLC1A2, IL1R2, FAM20A, RETN | | | | | | | | | |
| RSAD2, OAS1, SIGLEC1, HERC6, | 0.882 | 0.872 | 0.891 | 0.920 | 0.864 | 0.976 | 0.961 | 0.903 | 1 |
| IL1R2, OLAH, FAM20A, MMP8 | | | | | | | | | |
| IFI27, OAS1, IFIT1, IFI44L, ISG15, | 0.882 | 0.873 | 0.891 | 0.923 | 0.868 | 0.978 | 0.937 | 0.875 | 0.998 |
| HERC6, OLAH, RETN | | | | | | | | | |
| RSAD2, IFI27, OAS1, IFIT1, | 0.882 | 0.873 | 0.891 | 0.933 | 0.886 | 0.981 | 0.945 | 0.878 | 1 |
| SIGLEC1, SLC1A2, IL1R2, FAM20A | | | | | | | | | |
| IFI27, IFIT1, SIGLEC1, ISG15, | 0.882 | 0.872 | 0.891 | 0.930 | 0.878 | 0.981 | 0.941 | 0.869 | 1 |
| HERC6, SLC1A2, FAM20A, RETN | | | | | | | | | |
| RSAD2, IFI27, OAS1, SIGLEC1, | 0.882 | 0.872 | 0.891 | 0.922 | 0.863 | 0.980 | 0.949 | 0.889 | 1 |
| ISG15, HERC6, RETN, MMP8 | | | | | | | | | |
| IFI27, OAS1, HERC6, SLC1A2, | 0.882 | 0.872 | 0.891 | 0.932 | 0.883 | 0.981 | 0.916 | 0.830 | 1 |
| IL1R2, OLAH, FAM20A, RETN | | | | | | | | | |
| IFI27, OAS1, IFIT1, SIGLEC1, | 0.882 | 0.872 | 0.891 | 0.931 | 0.883 | 0.979 | 0.941 | 0.879 | 1 |
| HERC6, SLC1A2, OLAH, RETN | | | | | | | | | |
| IFI27, OAS1, SIGLEC1, ISG15, | 0.882 | 0.872 | 0.891 | 0.933 | 0.885 | 0.980 | 0.945 | 0.876 | 1 |
| SLC1A2, IL1R2, FAM20A, RETN | | | | | | | | | |
| IFI27, IFI44L, ISG15, HERC6, | 0.882 | 0.872 | 0.891 | 0.930 | 0.877 | 0.982 | 0.917 | 0.834 | 1 |
| SLC1A2, OLAH, FAM20A, RETN | | | | | | | | | |
| IFI27, IFI44L, SIGLEC1, HERC6, | 0.882 | 0.872 | 0.892 | 0.930 | 0.877 | 0.983 | 0.946 | 0.878 | 1 |
| SLC1A2, OLAH, FAM20A, RETN | | | | | | | | | |
| IFI27, OAS1, IFI44L, SIGLEC1, | 0.882 | 0.873 | 0.890 | 0.926 | 0.875 | 0.977 | 0.941 | 0.886 | 0.996 |
| ISG15, HERC6, OLAH, MMP8 | | | | | | | | | |
| RSAD2, IFI27, OAS1, IFIT1, | 0.882 | 0.873 | 0.890 | 0.924 | 0.875 | 0.974 | 0.942 | 0.890 | 0.995 |
| SIGLEC1, HERC6, IL1R2, OLAH | | | | | | | | | |
| IFI27, IFIT1, IFI44L, SLC1A2, | 0.882 | 0.872 | 0.892 | 0.928 | 0.873 | 0.983 | 0.928 | 0.850 | 1 |
| OLAH, FAM20A, RETN, MMP8 | | | | | | | | | |
| RSAD2, IFI27, OAS1, SIGLEC1, | 0.882 | 0.873 | 0.890 | 0.924 | 0.872 | 0.977 | 0.953 | 0.900 | 1 |
| ISG15, HERC6, IL1R2, RETN | | | | | | | | | |
| OAS1, IFIT1, SIGLEC1, ISG15, | 0.882 | 0.873 | 0.891 | 0.920 | 0.863 | 0.977 | 0.966 | 0.913 | 1 |
| IL1R2, OLAH, FAM20A, MMP8 | | | | | | | | | |
| RSAD2, IFI27, IFI44L, SIGLEC1, | 0.882 | 0.872 | 0.891 | 0.932 | 0.885 | 0.980 | 0.945 | 0.874 | 1 |
| ISG15, SLC1A2, IL1R2, FAM20A | | | | | | | | | |
| RSAD2, IFI27, IFIT1, IFI44L, | 0.882 | 0.872 | 0.891 | 0.929 | 0.875 | 0.982 | 0.934 | 0.856 | 1 |
| IL1R2, FAM20A, RETN, MMP8 | | | | | | | | | |
| IFI27, IFIT1, SIGLEC1, ISG15, | 0.882 | 0.873 | 0.890 | 0.925 | 0.876 | 0.975 | 0.945 | 0.893 | 0.996 |
| HERC6, IL1R2, OLAH, MMP8 | | | | | | | | | |
| RSAD2, IFI27, OAS1, IFI44L, | 0.881 | 0.872 | 0.891 | 0.925 | 0.868 | 0.982 | 0.930 | 0.858 | 1 |
| IL1R2, OLAH, FAM20A, RETN | | | | | | | | | |
| RSAD2, IFI27, IFI44L, SIGLEC1, | 0.881 | 0.872 | 0.891 | 0.927 | 0.871 | 0.983 | 0.943 | 0.878 | 1 |
| SLC1A2, OLAH, FAM20A, RETN | | | | | | | | | |
| OAS1, IFIT1, IFI44L, SIGLEC1, | 0.881 | 0.872 | 0.891 | 0.920 | 0.863 | 0.977 | 0.966 | 0.915 | 1 |
| IL1R2, OLAH, FAM20A, MMP8 | | | | | | | | | |
| RSAD2, IFI27, IFIT1, ISG15, | 0.881 | 0.872 | 0.891 | 0.924 | 0.868 | 0.981 | 0.945 | 0.889 | 1 |
| HERC6, OLAH, RETN, MMP8 | | | | | | | | | |
| RSAD2, IFI27, ISG15, SLC1A2, | 0.881 | 0.872 | 0.891 | 0.930 | 0.880 | 0.981 | 0.916 | 0.833 | 1.000 |
| IL1R2, OLAH, FAM20A, RETN | | | | | | | | | |
| RSAD2, IFI27, SIGLEC1, HERC6, | 0.881 | 0.871 | 0.892 | 0.929 | 0.875 | 0.982 | 0.946 | 0.872 | 1 |
| SLC1A2, FAM20A, RETN, MMP8 | | | | | | | | | |
| RSAD2, IFI27, OAS1, ISG15, | 0.881 | 0.871 | 0.892 | 0.926 | 0.870 | 0.983 | 0.935 | 0.857 | 1 |
| HERC6, FAM20A, RETN, MMP8 | | | | | | | | | |
| RSAD2, IFI27, OAS1, IFI44L, | 0.881 | 0.872 | 0.891 | 0.930 | 0.877 | 0.983 | 0.918 | 0.836 | 1 |
| ISG15, SLC1A2, OLAH, FAM20A | | | | | | | | | |
| IFI27, OAS1, IFI44L, ISG15, | 0.881 | 0.873 | 0.890 | 0.922 | 0.867 | 0.977 | 0.938 | 0.877 | 0.999 |
| HERC6, IL1R2, OLAH, RETN | | | | | | | | | |
| RSAD2, IFI27, OAS1, IFIT1, | 0.881 | 0.872 | 0.891 | 0.922 | 0.861 | 0.982 | 0.936 | 0.867 | 1 |
| IFI44L, OLAH, FAM20A, RETN | | | | | | | | | |
| IFI27, IFIT1, IFI44L, HERC6, | 0.881 | 0.872 | 0.891 | 0.931 | 0.880 | 0.983 | 0.925 | 0.845 | 1 |
| SLC1A2, OLAH, FAM20A, RETN | | | | | | | | | |
| IFI27, IFI44L, SIGLEC1, ISG15, | 0.881 | 0.872 | 0.890 | 0.924 | 0.870 | 0.978 | 0.954 | 0.902 | 1 |
| IL1R2, OLAH, RETN, MMP8 | | | | | | | | | |
| RSAD2, IFI27, OAS1, IFI44L, | 0.881 | 0.872 | 0.890 | 0.922 | 0.866 | 0.978 | 0.941 | 0.882 | 1 |
| ISG15, HERC6, OLAH, RETN | | | | | | | | | |
| RSAD2, IFI27, SIGLEC1, ISG15, | 0.881 | 0.873 | 0.890 | 0.925 | 0.876 | 0.974 | 0.943 | 0.892 | 0.995 |
| HERC6, IL1R2, OLAH, MMP8 | | | | | | | | | |
| IFI27, OAS1, IFIT1, IFI44L, | 0.881 | 0.872 | 0.890 | 0.923 | 0.869 | 0.977 | 0.938 | 0.877 | 0.999 |
| HERC6, IL1R2, OLAH, RETN | | | | | | | | | |
| IFI27, IFIT1, ISG15, HERC6, | 0.881 | 0.871 | 0.891 | 0.928 | 0.875 | 0.981 | 0.915 | 0.829 | 1 |
| SLC1A2, OLAH, FAM20A, RETN | | | | | | | | | |
| RSAD2, IFI27, OAS1, SIGLEC1, | 0.881 | 0.872 | 0.890 | 0.923 | 0.871 | 0.975 | 0.937 | 0.879 | 0.995 |
| ISG15, HERC6, OLAH, MMP8 | | | | | | | | | |
| IFI27, IFIT1, IFI44L, SIGLEC1, | 0.881 | 0.873 | 0.890 | 0.924 | 0.871 | 0.977 | 0.945 | 0.893 | 0.996 |
| ISG15, HERC6, IL1R2, RETN | | | | | | | | | |
| RSAD2, IFI27, OAS1, SIGLEC1, | 0.881 | 0.872 | 0.891 | 0.933 | 0.886 | 0.980 | 0.945 | 0.875 | 1 |
| ISG15, SLC1A2, IL1R2, FAM20A | | | | | | | | | |
| IFI27, ISG15, HERC6, SLC1A2, | 0.881 | 0.872 | 0.891 | 0.931 | 0.882 | 0.981 | 0.916 | 0.832 | 1 |
| IL1R2, OLAH, FAM20A, RETN | | | | | | | | | |
| IFI27, OAS1, IFIT1, IFI44L, | 0.881 | 0.871 | 0.891 | 0.923 | 0.865 | 0.982 | 0.934 | 0.853 | 1 |
| HERC6, FAM20A, RETN, MMP8 | | | | | | | | | |
| IFI27, IFI44L, HERC6, SLC1A2, | 0.881 | 0.871 | 0.891 | 0.929 | 0.875 | 0.983 | 0.927 | 0.848 | 1 |
| OLAH, FAM20A, RETN, MMP8 | | | | | | | | | |
| RSAD2, IFI27, IFIT1, SIGLEC1, | 0.881 | 0.872 | 0.890 | 0.934 | 0.886 | 0.981 | 0.943 | 0.874 | 1 |
| SLC1A2, IL1R2, FAM20A, RETN | | | | | | | | | |
| IFI27, OAS1, SLC1A2, IL1R2, | 0.881 | 0.872 | 0.891 | 0.933 | 0.884 | 0.983 | 0.923 | 0.843 | 1 |
| OLAH, FAM20A, RETN, MMP8 | | | | | | | | | |
| RSAD2, IFI27, OAS1, IFI44L, | 0.881 | 0.872 | 0.891 | 0.922 | 0.862 | 0.981 | 0.935 | 0.867 | 1 |
| ISG15, OLAH, FAM20A, RETN | | | | | | | | | |
| RSAD2, IFI27, SIGLEC1, ISG15, | 0.881 | 0.872 | 0.890 | 0.922 | 0.868 | 0.977 | 0.943 | 0.887 | 1.000 |
| HERC6, IL1R2, OLAH, RETN | | | | | | | | | |
| RSAD2, IFI27, OAS1, IFIT1, | 0.881 | 0.873 | 0.890 | 0.927 | 0.876 | 0.977 | 0.947 | 0.895 | 0.998 |
| IFI44L, SIGLEC1, HERC6, OLAH | | | | | | | | | |
| RSAD2, IFI27, OAS1, ISG15, | 0.881 | 0.872 | 0.890 | 0.925 | 0.876 | 0.975 | 0.938 | 0.880 | 0.996 |
| HERC6, IL1R2, OLAH, MMP8 | | | | | | | | | |
| RSAD2, IFI27, OAS1, IFIT1, ISG15, | 0.881 | 0.872 | 0.891 | 0.920 | 0.860 | 0.980 | 0.934 | 0.866 | 1 |
| OLAH, FAM20A, RETN | | | | | | | | | |
| RSAD2, IFI27, IFIT1, IFI44L, | 0.881 | 0.873 | 0.890 | 0.924 | 0.871 | 0.977 | 0.947 | 0.897 | 0.997 |
| SIGLEC1, HERC6, OLAH, MMP8 | | | | | | | | | |
| IFI27, IFIT1, IFI44L, HERC6, | 0.881 | 0.872 | 0.890 | 0.922 | 0.864 | 0.979 | 0.947 | 0.892 | 1 |
| IL1R2, OLAH, RETN, MMP8 | | | | | | | | | |
| IFIT1, IFI44L, SIGLEC1, ISG15, | 0.881 | 0.872 | 0.890 | 0.924 | 0.872 | 0.977 | 0.941 | 0.877 | 1 |
| SLC1A2, OLAH, FAM20A, MMP8 | | | | | | | | | |
| RSAD2, IFI27, IFIT1, IFI44L, | 0.881 | 0.872 | 0.891 | 0.920 | 0.860 | 0.981 | 0.935 | 0.865 | 1 |
| ISG15, OLAH, FAM20A, RETN | | | | | | | | | |
| RSAD2, IFI27, SIGLEC1, HERC6, | 0.881 | 0.872 | 0.891 | 0.932 | 0.883 | 0.981 | 0.952 | 0.900 | 1 |
| SLC1A2, OLAH, RETN, MMP8 | | | | | | | | | |
| RSAD2, IFI27, OAS1, IFIT1, | 0.881 | 0.872 | 0.891 | 0.929 | 0.874 | 0.983 | 0.925 | 0.847 | 1 |
| IFI44L, SLC1A2, OLAH, FAM20A | | | | | | | | | |
| RSAD2, IFI27, OAS1, IFI44L, | 0.881 | 0.872 | 0.890 | 0.923 | 0.867 | 0.978 | 0.939 | 0.879 | 0.999 |
| HERC6, IL1R2, OLAH, RETN | | | | | | | | | |
| IFI27, SIGLEC1, ISG15, HERC6, | 0.881 | 0.872 | 0.890 | 0.922 | 0.865 | 0.978 | 0.958 | 0.909 | 1 |
| IL1R2, OLAH, RETN, MMP8 | | | | | | | | | |
| RSAD2, IFI27, SIGLEC1, ISG15, | 0.881 | 0.871 | 0.891 | 0.933 | 0.886 | 0.980 | 0.943 | 0.873 | 1 |
| SLC1A2, IL1R2, FAM20A, RETN | | | | | | | | | |
| RSAD2, IFI27, OAS1, IFI44L, | 0.881 | 0.872 | 0.890 | 0.926 | 0.874 | 0.978 | 0.943 | 0.890 | 0.997 |
| SIGLEC1, ISG15, HERC6, OLAH | | | | | | | | | |
| IFI27, OAS1, IFIT1, HERC6, | 0.881 | 0.871 | 0.891 | 0.929 | 0.878 | 0.979 | 0.940 | 0.876 | 1 |
| SLC1A2, OLAH, RETN, MMP8 | | | | | | | | | |
| IFIT1, IFI44L, SIGLEC1, ISG15, | 0.881 | 0.872 | 0.890 | 0.918 | 0.860 | 0.976 | 0.951 | 0.894 | 1 |
| HERC6, IL1R2, OLAH, FAM20A | | | | | | | | | |
| RSAD2, IFI27, IFIT1, SIGLEC1, | 0.881 | 0.872 | 0.890 | 0.926 | 0.877 | 0.975 | 0.942 | 0.890 | 0.995 |
| HERC6, IL1R2, OLAH, MMP8 | | | | | | | | | |
| RSAD2, IFI27, IFI44L, SIGLEC1, | 0.881 | 0.872 | 0.890 | 0.924 | 0.872 | 0.977 | 0.952 | 0.906 | 0.998 |
| HERC6, IL1R2, RETN, MMP8 | | | | | | | | | |
| IFIT1, IFI44L, SIGLEC1, ISG15, | 0.881 | 0.872 | 0.890 | 0.920 | 0.864 | 0.976 | 0.965 | 0.914 | 1 |
| IL1R2, OLAH, FAM20A, MMP8 | | | | | | | | | |
| IFI27, OAS1, IFIT1, SIGLEC1, | 0.881 | 0.872 | 0.890 | 0.921 | 0.865 | 0.978 | 0.963 | 0.914 | 1 |
| IL1R2, OLAH, RETN, MMP8 | | | | | | | | | |
| IFI27, IFIT1, ISG15, HERC6, IL1R2, | 0.881 | 0.872 | 0.890 | 0.922 | 0.865 | 0.979 | 0.947 | 0.892 | 1 |
| OLAH, RETN, MMP8 | | | | | | | | | |
| RSAD2, IFI27, IFIT1, IFI44L, | 0.881 | 0.873 | 0.889 | 0.923 | 0.870 | 0.976 | 0.945 | 0.893 | 0.996 |
| SIGLEC1, HERC6, IL1R2, RETN | | | | | | | | | |
| RSAD2, IFI27, OAS1, IFI44L, | 0.881 | 0.870 | 0.891 | 0.925 | 0.867 | 0.983 | 0.948 | 0.876 | 1 |
| SIGLEC1, FAM20A, RETN, MMP8 | | | | | | | | | |
| RSAD2, IFI27, IFI44L, HERC6, | 0.881 | 0.871 | 0.891 | 0.931 | 0.881 | 0.982 | 0.922 | 0.842 | 1 |
| SLC1A2, OLAH, FAM20A, RETN | | | | | | | | | |
| RSAD2, IFI27, OAS1, SIGLEC1, | 0.881 | 0.872 | 0.890 | 0.929 | 0.878 | 0.980 | 0.945 | 0.883 | 1 |
| HERC6, SLC1A2, OLAH, RETN | | | | | | | | | |
| IFI27, OAS1, IFIT1, IFI44L, ISG15, | 0.881 | 0.871 | 0.891 | 0.926 | 0.873 | 0.978 | 0.933 | 0.854 | 1 |
| IL1R2, FAM20A, MMP8 | | | | | | | | | |
| IFI27, IFIT1, IFI44L, SIGLEC1, | 0.881 | 0.872 | 0.889 | 0.924 | 0.875 | 0.974 | 0.945 | 0.893 | 0.996 |
| ISG15, IL1R2, OLAH, MMP8 | | | | | | | | | |
| IFI27, OAS1, IFIT1, IFI44L, | 0.881 | 0.872 | 0.890 | 0.921 | 0.863 | 0.979 | 0.955 | 0.899 | 1 |
| SIGLEC1, OLAH, RETN, MMP8 | | | | | | | | | |
| RSAD2, IFI27, OAS1, SIGLEC1, | 0.881 | 0.871 | 0.891 | 0.930 | 0.881 | 0.980 | 0.942 | 0.871 | 1 |
| SLC1A2, IL1R2, FAM20A, RETN | | | | | | | | | |
| IFI27, OAS1, SIGLEC1, HERC6, | 0.881 | 0.872 | 0.890 | 0.929 | 0.881 | 0.977 | 0.943 | 0.887 | 1 |
| SLC1A2, IL1R2, OLAH, MMP8 | | | | | | | | | |
| IFI27, OAS1, IFIT1, ISG15, HERC6, | 0.881 | 0.871 | 0.891 | 0.926 | 0.869 | 0.982 | 0.935 | 0.857 | 1 |
| FAM20A, RETN, MMP8 | | | | | | | | | |
| IFI44L, SIGLEC1, ISG15, HERC6, | 0.881 | 0.872 | 0.890 | 0.916 | 0.858 | 0.974 | 0.961 | 0.903 | 1 |
| IL1R2, OLAH, FAM20A, MMP8 | | | | | | | | | |
| RSAD2, IFI27, IFI44L, ISG15, | 0.881 | 0.872 | 0.890 | 0.923 | 0.866 | 0.980 | 0.943 | 0.887 | 1.000 |
| HERC6, OLAH, RETN, MMP8 | | | | | | | | | |
| RSAD2, IFI27, OAS1, IFI44L, | 0.881 | 0.872 | 0.890 | 0.928 | 0.879 | 0.977 | 0.935 | 0.875 | 0.995 |
| HERC6, IL1R2, OLAH, MMP8 | | | | | | | | | |
| IFI27, IFIT1, ISG15, SLC1A2, | 0.881 | 0.871 | 0.890 | 0.929 | 0.878 | 0.980 | 0.920 | 0.839 | 1 |
| IL1R2, OLAH, FAM20A, RETN | | | | | | | | | |
| IFI27, OAS1, IFIT1, ISG15, | 0.881 | 0.871 | 0.890 | 0.929 | 0.877 | 0.980 | 0.921 | 0.841 | 1 |
| SLC1A2, IL1R2, OLAH, FAM20A | | | | | | | | | |
| RSAD2, IFI27, IFIT1, ISG15, | 0.881 | 0.872 | 0.890 | 0.929 | 0.878 | 0.980 | 0.918 | 0.837 | 1.000 |
| SLC1A2, IL1R2, OLAH, FAM20A | | | | | | | | | |
| IFI27, IFI44L, SIGLEC1, ISG15, | 0.881 | 0.872 | 0.889 | 0.930 | 0.882 | 0.978 | 0.946 | 0.892 | 0.999 |
| SLC1A2, IL1R2, OLAH, MMP8 | | | | | | | | | |
| RSAD2, IFI27, ISG15, SLC1A2, | 0.881 | 0.871 | 0.891 | 0.928 | 0.874 | 0.982 | 0.924 | 0.843 | 1 |
| OLAH, FAM20A, RETN, MMP8 | | | | | | | | | |
| RSAD2, IFI27, OAS1, IFIT1, ISG15, | 0.881 | 0.871 | 0.890 | 0.925 | 0.870 | 0.980 | 0.923 | 0.843 | 1 |
| SLC1A2, OLAH, FAM20A | | | | | | | | | |
| IFI27, IFIT1, IFI44L, SIGLEC1, | 0.881 | 0.872 | 0.890 | 0.924 | 0.870 | 0.979 | 0.955 | 0.904 | 1 |
| IL1R2, OLAH, RETN, MMP8 | | | | | | | | | |
| IFI27, IFI44L, SIGLEC1, HERC6, | 0.881 | 0.871 | 0.890 | 0.931 | 0.882 | 0.980 | 0.948 | 0.879 | 1 |
| SLC1A2, IL1R2, FAM20A, RETN | | | | | | | | | |
| RSAD2, IFI27, IFI44L, SIGLEC1, | 0.881 | 0.872 | 0.889 | 0.925 | 0.873 | 0.978 | 0.946 | 0.894 | 0.998 |
| ISG15, HERC6, OLAH, MMP8 | | | | | | | | | |
| IFI27, IFIT1, ISG15, SLC1A2, | 0.881 | 0.870 | 0.891 | 0.927 | 0.872 | 0.981 | 0.925 | 0.849 | 1 |
| OLAH, FAM20A, RETN, MMP8 | | | | | | | | | |
| RSAD2, IFI27, IFIT1, IFI44L, | 0.881 | 0.871 | 0.890 | 0.925 | 0.868 | 0.982 | 0.932 | 0.860 | 1 |
| IL1R2, OLAH, FAM20A, RETN | | | | | | | | | |
| IFI27, OAS1, IFI44L, ISG15, | 0.881 | 0.872 | 0.889 | 0.927 | 0.878 | 0.976 | 0.934 | 0.874 | 0.993 |
| HERC6, IL1R2, OLAH, MMP8 | | | | | | | | | |
| IFI27, OAS1, IFI44L, ISG15, | 0.881 | 0.871 | 0.890 | 0.930 | 0.877 | 0.983 | 0.917 | 0.835 | 1.000 |
| SLC1A2, OLAH, FAM20A, RETN | | | | | | | | | |
| RSAD2, IFI27, OAS1, IFIT1, | 0.881 | 0.872 | 0.890 | 0.915 | 0.850 | 0.979 | 0.943 | 0.873 | 1 |
| IFI44L, SIGLEC1, ISG15, FAM20A | | | | | | | | | |
| IFI27, OAS1, SIGLEC1, ISG15, | 0.881 | 0.871 | 0.890 | 0.930 | 0.881 | 0.980 | 0.937 | 0.877 | 0.997 |
| HERC6, SLC1A2, OLAH, MMP8 | | | | | | | | | |
| RSAD2, IFI27, OAS1, IFIT1, | 0.881 | 0.872 | 0.890 | 0.925 | 0.876 | 0.974 | 0.939 | 0.882 | 0.996 |
| HERC6, IL1R2, OLAH, MMP8 | | | | | | | | | |
| RSAD2, IFI27, IFIT1, SLC1A2, | 0.881 | 0.871 | 0.890 | 0.926 | 0.871 | 0.981 | 0.927 | 0.852 | 1 |
| OLAH, FAM20A, RETN, MMP8 | | | | | | | | | |
| RSAD2, IFIT1, SIGLEC1, HERC6, | 0.881 | 0.872 | 0.889 | 0.923 | 0.868 | 0.979 | 0.960 | 0.900 | 1 |
| IL1R2, FAM20A, RETN, MMP8 | | | | | | | | | |
| IFI27, IFIT1, IFI44L, SIGLEC1, | 0.881 | 0.871 | 0.890 | 0.925 | 0.870 | 0.980 | 0.938 | 0.862 | 1 |
| ISG15, SLC1A2, FAM20A, RETN | | | | | | | | | |
| IFI27, OAS1, IFIT1, SIGLEC1, | 0.881 | 0.871 | 0.890 | 0.930 | 0.880 | 0.979 | 0.937 | 0.877 | 0.997 |
| ISG15, HERC6, SLC1A2, OLAH | | | | | | | | | |
| RSAD2, IFI27, OAS1, IFI44L, | 0.881 | 0.871 | 0.890 | 0.919 | 0.861 | 0.978 | 0.941 | 0.875 | 1 |
| SIGLEC1, HERC6, RETN, MMP8 | | | | | | | | | |
| RSAD2, IFI27, IFIT1, HERC6, | 0.881 | 0.871 | 0.890 | 0.930 | 0.879 | 0.982 | 0.918 | 0.834 | 1 |
| SLC1A2, OLAH, FAM20A, RETN | | | | | | | | | |
| RSAD2, IFI27, IFIT1, IFI44L, | 0.881 | 0.871 | 0.890 | 0.926 | 0.873 | 0.978 | 0.933 | 0.854 | 1 |
| ISG15, IL1R2, FAM20A, MMP8 | | | | | | | | | |
| RSAD2, IFI27, IFIT1, SIGLEC1, | 0.880 | 0.872 | 0.889 | 0.924 | 0.871 | 0.976 | 0.959 | 0.916 | 1 |
| HERC6, IL1R2, RETN, MMP8 | | | | | | | | | |
| IFI27, OAS1, IFI44L, SIGLEC1, | 0.880 | 0.870 | 0.891 | 0.923 | 0.864 | 0.982 | 0.947 | 0.875 | 1 |
| ISG15, FAM20A, RETN, MMP8 | | | | | | | | | |
| IFI27, OAS1, SIGLEC1, ISG15, | 0.880 | 0.871 | 0.890 | 0.928 | 0.877 | 0.978 | 0.938 | 0.874 | 1 |
| HERC6, SLC1A2, OLAH, RETN | | | | | | | | | |
| IFI27, OAS1, IFI44L, SIGLEC1, | 0.880 | 0.871 | 0.890 | 0.928 | 0.875 | 0.980 | 0.938 | 0.864 | 1 |
| HERC6, SLC1A2, FAM20A, RETN | | | | | | | | | |
| IFI27, OAS1, IFIT1, SLC1A2, | 0.880 | 0.870 | 0.891 | 0.926 | 0.870 | 0.981 | 0.927 | 0.852 | 1 |
| OLAH, FAM20A, RETN, MMP8 | | | | | | | | | |
| RSAD2, IFI27, IFIT1, ISG15, IL1R2, | 0.880 | 0.871 | 0.890 | 0.930 | 0.877 | 0.982 | 0.934 | 0.856 | 1 |
| FAM20A, RETN, MMP8 | | | | | | | | | |
| RSAD2, OAS1, IFI44L, SIGLEC1, | 0.880 | 0.871 | 0.890 | 0.919 | 0.862 | 0.975 | 0.960 | 0.900 | 1 |
| HERC6, IL1R2, FAM20A, MMP8 | | | | | | | | | |
| IFI27, OAS1, SIGLEC1, HERC6, | 0.880 | 0.871 | 0.890 | 0.928 | 0.877 | 0.979 | 0.942 | 0.880 | 1 |
| SLC1A2, IL1R2, OLAH, RETN | | | | | | | | | |
| RSAD2, IFI27, OAS1, IFIT1, ISG15, | 0.880 | 0.871 | 0.890 | 0.926 | 0.873 | 0.978 | 0.934 | 0.856 | 1 |
| IL1R2, FAM20A, MMP8 | | | | | | | | | |
| IFIT1, IFI44L, SIGLEC1, HERC6, | 0.880 | 0.870 | 0.891 | 0.929 | 0.880 | 0.979 | 0.941 | 0.872 | 1 |
| SLC1A2, OLAH, FAM20A, MMP8 | | | | | | | | | |
| IFI27, OAS1, IFIT1, ISG15, | 0.880 | 0.871 | 0.890 | 0.925 | 0.869 | 0.981 | 0.925 | 0.849 | 1 |
| SLC1A2, OLAH, FAM20A, RETN | | | | | | | | | |
| IFI27, IFI44L, SIGLEC1, ISG15, | 0.880 | 0.872 | 0.889 | 0.924 | 0.871 | 0.977 | 0.951 | 0.905 | 0.998 |
| HERC6, IL1R2, RETN, MMP8 | | | | | | | | | |
| IFI27, OAS1, IFI44L, SIGLEC1, | 0.880 | 0.871 | 0.889 | 0.924 | 0.869 | 0.979 | 0.958 | 0.908 | 1 |
| IL1R2, OLAH, RETN, MMP8 | | | | | | | | | |
| IFI27, OAS1, IFIT1, IFI44L, | 0.880 | 0.872 | 0.889 | 0.924 | 0.874 | 0.974 | 0.949 | 0.900 | 0.998 |
| SIGLEC1, IL1R2, OLAH, MMP8 | | | | | | | | | |
| IFIT1, IFI44L, SIGLEC1, HERC6, | 0.880 | 0.870 | 0.890 | 0.922 | 0.864 | 0.980 | 0.952 | 0.895 | 1 |
| OLAH, FAM20A, RETN, MMP8 | | | | | | | | | |
| IFI27, OAS1, ISG15, SLC1A2, | 0.880 | 0.871 | 0.890 | 0.929 | 0.878 | 0.980 | 0.916 | 0.834 | 0.999 |
| IL1R2, OLAH, FAM20A, RETN | | | | | | | | | |
| RSAD2, IFI27, IFIT1, IFI44L, | 0.880 | 0.871 | 0.889 | 0.926 | 0.872 | 0.981 | 0.922 | 0.841 | 1 |
| SLC1A2, IL1R2, OLAH, FAM20A | | | | | | | | | |
| RSAD2, IFI27, IFI44L, ISG15, | 0.880 | 0.872 | 0.889 | 0.924 | 0.871 | 0.978 | 0.937 | 0.878 | 0.996 |
| HERC6, IL1R2, OLAH, RETN | | | | | | | | | |
| IFIT1, IFI44L, SIGLEC1, SLC1A2, | 0.880 | 0.872 | 0.889 | 0.928 | 0.880 | 0.976 | 0.963 | 0.908 | 1 |
| IL1R2, OLAH, FAM20A, MMP8 | | | | | | | | | |
| IFI27, OAS1, IFIT1, IFI44L, | 0.880 | 0.871 | 0.890 | 0.930 | 0.876 | 0.983 | 0.921 | 0.839 | 1 |
| SLC1A2, IL1R2, OLAH, FAM20A | | | | | | | | | |
| OAS1, IFIT1, IFI44L, SIGLEC1, | 0.880 | 0.871 | 0.889 | 0.916 | 0.858 | 0.975 | 0.951 | 0.894 | 1 |
| HERC6, IL1R2, OLAH, FAM20A | | | | | | | | | |
| OAS1, IFIT1, SIGLEC1, HERC6, | 0.880 | 0.871 | 0.890 | 0.928 | 0.882 | 0.974 | 0.959 | 0.895 | 1 |
| SLC1A2, IL1R2, FAM20A, MMP8 | | | | | | | | | |
| RSAD2, IFI27, OAS1, SLC1A2, | 0.880 | 0.870 | 0.890 | 0.927 | 0.872 | 0.982 | 0.924 | 0.844 | 1 |
| OLAH, FAM20A, RETN, MMP8 | | | | | | | | | |
| IFI27, OAS1, SIGLEC1, ISG15, | 0.880 | 0.871 | 0.890 | 0.928 | 0.879 | 0.978 | 0.939 | 0.880 | 0.998 |
| HERC6, SLC1A2, IL1R2, OLAH | | | | | | | | | |
| IFI27, IFIT1, IFI44L, ISG15, | 0.880 | 0.870 | 0.890 | 0.921 | 0.863 | 0.980 | 0.935 | 0.856 | 1 |
| HERC6, FAM20A, RETN, MMP8 | | | | | | | | | |
| IFI27, OAS1, SIGLEC1, ISG15, | 0.880 | 0.870 | 0.890 | 0.927 | 0.874 | 0.980 | 0.937 | 0.863 | 1 |
| HERC6, SLC1A2, FAM20A, RETN | | | | | | | | | |
| RSAD2, IFIT1, IFI44L, SIGLEC1, | 0.880 | 0.871 | 0.889 | 0.921 | 0.864 | 0.978 | 0.966 | 0.915 | 1 |
| IL1R2, OLAH, FAM20A, MMP8 | | | | | | | | | |
| RSAD2, IFI27, OAS1, IFI44L, | 0.880 | 0.870 | 0.890 | 0.925 | 0.872 | 0.978 | 0.930 | 0.847 | 1 |
| ISG15, IL1R2, FAM20A, MMP8 | | | | | | | | | |
| IFI27, IFIT1, IFI44L, SLC1A2, | 0.880 | 0.871 | 0.889 | 0.928 | 0.874 | 0.982 | 0.922 | 0.841 | 1 |
| IL1R2, OLAH, FAM20A, RETN | | | | | | | | | |
| IFI27, OAS1, IFI44L, SIGLEC1, | 0.880 | 0.872 | 0.888 | 0.925 | 0.876 | 0.975 | 0.941 | 0.888 | 0.995 |
| ISG15, IL1R2, OLAH, MMP8 | | | | | | | | | |
| RSAD2, IFI27, OAS1, HERC6, | 0.880 | 0.871 | 0.890 | 0.930 | 0.880 | 0.981 | 0.941 | 0.877 | 1 |
| SLC1A2, OLAH, RETN, MMP8 | | | | | | | | | |
| IFI27, OAS1, IFIT1, SIGLEC1, | 0.880 | 0.870 | 0.890 | 0.927 | 0.872 | 0.982 | 0.942 | 0.866 | 1 |
| SLC1A2, FAM20A, RETN, MMP8 | | | | | | | | | |
| RSAD2, IFI27, OAS1, IFI44L, | 0.880 | 0.870 | 0.890 | 0.918 | 0.858 | 0.978 | 0.947 | 0.879 | 1 |
| SIGLEC1, ISG15, FAM20A, RETN | | | | | | | | | |
| OAS1, IFIT1, SIGLEC1, HERC6, | 0.880 | 0.871 | 0.889 | 0.920 | 0.862 | 0.979 | 0.952 | 0.895 | 1 |
| OLAH, FAM20A, RETN, MMP8 | | | | | | | | | |
| IFI27, OAS1, HERC6, SLC1A2, | 0.880 | 0.871 | 0.889 | 0.935 | 0.887 | 0.982 | 0.924 | 0.837 | 1 |
| IL1R2, FAM20A, RETN, MMP8 | | | | | | | | | |
| RSAD2, IFI27, IFIT1, ISG15, | 0.880 | 0.871 | 0.889 | 0.922 | 0.867 | 0.977 | 0.937 | 0.876 | 0.998 |
| HERC6, IL1R2, OLAH, RETN | | | | | | | | | |
| RSAD2, IFI27, OAS1, IFIT1, | 0.880 | 0.871 | 0.889 | 0.925 | 0.873 | 0.976 | 0.935 | 0.876 | 0.994 |
| IFI44L, HERC6, OLAH, MMP8 | | | | | | | | | |
| RSAD2, IFIT1, SIGLEC1, ISG15, | 0.880 | 0.871 | 0.889 | 0.919 | 0.862 | 0.976 | 0.967 | 0.916 | 1 |
| IL1R2, OLAH, FAM20A, MMP8 | | | | | | | | | |
| IFI27, OAS1, HERC6, SLC1A2, | 0.880 | 0.871 | 0.889 | 0.928 | 0.878 | 0.979 | 0.938 | 0.874 | 1 |
| IL1R2, OLAH, RETN, MMP8 | | | | | | | | | |
| IFI27, OAS1, IFIT1, IFI44L, | 0.880 | 0.871 | 0.889 | 0.930 | 0.881 | 0.979 | 0.937 | 0.862 | 1 |
| SIGLEC1, HERC6, SLC1A2, FAM20A | | | | | | | | | |
| RSAD2, IFI27, OAS1, IFIT1, | 0.880 | 0.871 | 0.889 | 0.924 | 0.873 | 0.976 | 0.938 | 0.881 | 0.995 |
| SIGLEC1, ISG15, HERC6, OLAH | | | | | | | | | |
| IFI27, OAS1, IFI44L, ISG15, IL1R2, | 0.880 | 0.870 | 0.890 | 0.929 | 0.877 | 0.982 | 0.935 | 0.856 | 1 |
| FAM20A, RETN, MMP8 | | | | | | | | | |
| RSAD2, IFI27, OAS1, IFIT1, | 0.880 | 0.871 | 0.889 | 0.927 | 0.873 | 0.981 | 0.922 | 0.840 | 1 |
| SLC1A2, IL1R2, OLAH, FAM20A | | | | | | | | | |
| IFI27, OAS1, IFIT1, SIGLEC1, | 0.880 | 0.870 | 0.890 | 0.928 | 0.878 | 0.978 | 0.939 | 0.880 | 0.999 |
| HERC6, SLC1A2, OLAH, MMP8 | | | | | | | | | |
| RSAD2, IFI27, IFI44L, SIGLEC1, | 0.880 | 0.870 | 0.890 | 0.931 | 0.881 | 0.981 | 0.945 | 0.875 | 1 |
| SLC1A2, IL1R2, FAM20A, RETN | | | | | | | | | |
| IFI27, IFIT1, SIGLEC1, ISG15, | 0.880 | 0.871 | 0.889 | 0.922 | 0.866 | 0.978 | 0.960 | 0.911 | 1 |
| IL1R2, OLAH, RETN, MMP8 | | | | | | | | | |
| IFIT1, SIGLEC1, HERC6, SLC1A2, | 0.880 | 0.870 | 0.890 | 0.927 | 0.879 | 0.974 | 0.960 | 0.896 | 1 |
| IL1R2, OLAH, FAM20A, MMP8 | | | | | | | | | |
| RSAD2, IFI27, IFI44L, SIGLEC1, | 0.880 | 0.869 | 0.890 | 0.924 | 0.866 | 0.982 | 0.947 | 0.875 | 1 |
| ISG15, FAM20A, RETN, MMP8 | | | | | | | | | |
| IFI27, OAS1, IFIT1, IFI44L, | 0.880 | 0.870 | 0.889 | 0.927 | 0.870 | 0.983 | 0.923 | 0.843 | 1 |
| SLC1A2, OLAH, FAM20A, RETN | | | | | | | | | |
| RSAD2, IFI27, IFIT1, SIGLEC1, | 0.880 | 0.871 | 0.888 | 0.925 | 0.875 | 0.975 | 0.939 | 0.885 | 0.993 |
| ISG15, HERC6, IL1R2, OLAH | | | | | | | | | |
| OAS1, IFIT1, SIGLEC1, IL1R2, | 0.880 | 0.871 | 0.889 | 0.921 | 0.864 | 0.978 | 0.967 | 0.916 | 1 |
| OLAH, FAM20A, RETN, MMP8 | | | | | | | | | |
| RSAD2, IFI27, OAS1, SIGLEC1, | 0.880 | 0.870 | 0.889 | 0.929 | 0.879 | 0.979 | 0.941 | 0.882 | 1 |
| HERC6, SLC1A2, OLAH, MMP8 | | | | | | | | | |
| RSAD2, IFI27, IFI44L, SLC1A2, | 0.880 | 0.870 | 0.890 | 0.929 | 0.875 | 0.983 | 0.928 | 0.853 | 1 |
| OLAH, FAM20A, RETN, MMP8 | | | | | | | | | |
| RSAD2, IFI27, OAS1, IFI44L, | 0.880 | 0.870 | 0.890 | 0.931 | 0.882 | 0.979 | 0.943 | 0.873 | 1 |
| SIGLEC1, SLC1A2, IL1R2, FAM20A | | | | | | | | | |
| RSAD2, IFI27, OAS1, IFIT1, | 0.880 | 0.871 | 0.889 | 0.923 | 0.868 | 0.978 | 0.945 | 0.886 | 1 |
| HERC6, IL1R2, RETN, MMP8 | | | | | | | | | |
| IFI27, OAS1, IFIT1, IFI44L, ISG15, | 0.880 | 0.871 | 0.889 | 0.924 | 0.871 | 0.977 | 0.930 | 0.852 | 1 |
| IL1R2, FAM20A, RETN | | | | | | | | | |
| IFI27, OAS1, IFI44L, SIGLEC1, | 0.880 | 0.870 | 0.889 | 0.931 | 0.884 | 0.979 | 0.938 | 0.874 | 1 |
| HERC6, SLC1A2, OLAH, RETN | | | | | | | | | |
| IFI27, IFIT1, SIGLEC1, ISG15, | 0.880 | 0.871 | 0.888 | 0.930 | 0.881 | 0.979 | 0.942 | 0.887 | 0.998 |
| SLC1A2, IL1R2, OLAH, MMP8 | | | | | | | | | |
| RSAD2, IFI27, OAS1, IFI44L, | 0.880 | 0.871 | 0.889 | 0.924 | 0.872 | 0.976 | 0.935 | 0.875 | 0.994 |
| ISG15, HERC6, OLAH, MMP8 | | | | | | | | | |
| RSAD2, IFI27, IFI44L, ISG15, | 0.880 | 0.870 | 0.889 | 0.929 | 0.875 | 0.982 | 0.916 | 0.834 | 0.999 |
| SLC1A2, OLAH, FAM20A, RETN | | | | | | | | | |
| RSAD2, OAS1, IFIT1, IFI44L, | 0.880 | 0.871 | 0.889 | 0.920 | 0.863 | 0.977 | 0.964 | 0.910 | 1 |
| SIGLEC1, IL1R2, FAM20A, MMP8 | | | | | | | | | |
| IFI27, OAS1, IFIT1, SIGLEC1, | 0.880 | 0.870 | 0.889 | 0.928 | 0.879 | 0.977 | 0.939 | 0.879 | 0.999 |
| HERC6, SLC1A2, IL1R2, OLAH | | | | | | | | | |
| IFI27, OAS1, IFIT1, IFI44L, | 0.880 | 0.872 | 0.888 | 0.924 | 0.878 | 0.970 | 0.940 | 0.888 | 0.993 |
| SIGLEC1, HERC6, IL1R2, MMP8 | | | | | | | | | |
| IFI27, OAS1, IFIT1, IFI44L, | 0.880 | 0.871 | 0.888 | 0.926 | 0.876 | 0.975 | 0.941 | 0.887 | 0.995 |
| HERC6, IL1R2, OLAH, MMP8 | | | | | | | | | |
| IFI27, IFIT1, HERC6, SLC1A2, | 0.880 | 0.870 | 0.890 | 0.929 | 0.877 | 0.981 | 0.922 | 0.839 | 1 |
| IL1R2, OLAH, FAM20A, RETN | | | | | | | | | |
| RSAD2, IFI27, IFIT1, IFI44L, | 0.880 | 0.871 | 0.888 | 0.922 | 0.867 | 0.977 | 0.936 | 0.876 | 0.996 |
| ISG15, HERC6, OLAH, RETN | | | | | | | | | |
| IFI27, OAS1, SIGLEC1, HERC6, | 0.880 | 0.871 | 0.889 | 0.928 | 0.881 | 0.976 | 0.955 | 0.903 | 1 |
| SLC1A2, IL1R2, RETN, MMP8 | | | | | | | | | |
| IFI27, OAS1, IFIT1, IFI44L, | 0.880 | 0.870 | 0.889 | 0.926 | 0.871 | 0.981 | 0.940 | 0.868 | 1 |
| SIGLEC1, SLC1A2, FAM20A, RETN | | | | | | | | | |
| RSAD2, IFI27, IFI44L, ISG15, | 0.880 | 0.871 | 0.888 | 0.926 | 0.877 | 0.976 | 0.937 | 0.879 | 0.995 |
| HERC6, IL1R2, OLAH, MMP8 | | | | | | | | | |
| RSAD2, IFI27, OAS1, IFI44L, | 0.880 | 0.871 | 0.888 | 0.924 | 0.873 | 0.974 | 0.948 | 0.898 | 0.998 |
| SIGLEC1, IL1R2, OLAH, MMP8 | | | | | | | | | |
| RSAD2, IFI27, OAS1, IFI44L, | 0.880 | 0.871 | 0.888 | 0.927 | 0.877 | 0.976 | 0.937 | 0.879 | 0.995 |
| ISG15, HERC6, IL1R2, OLAH | | | | | | | | | |
| IFI27, IFIT1, IFI44L, SIGLEC1, | 0.880 | 0.870 | 0.889 | 0.926 | 0.870 | 0.981 | 0.945 | 0.868 | 1 |
| SLC1A2, FAM20A, RETN, MMP8 | | | | | | | | | |
| IFI27, OAS1, IFIT1, SIGLEC1, | 0.880 | 0.870 | 0.889 | 0.920 | 0.861 | 0.979 | 0.961 | 0.910 | 1 |
| ISG15, OLAH, RETN, MMP8 | | | | | | | | | |
| RSAD2, IFI27, OAS1, IFIT1, | 0.880 | 0.871 | 0.889 | 0.919 | 0.859 | 0.978 | 0.934 | 0.865 | 1 |
| IFI44L, ISG15, HERC6, RETN | | | | | | | | | |
| IFI27, IFI44L, SIGLEC1, HERC6, | 0.880 | 0.870 | 0.889 | 0.926 | 0.873 | 0.979 | 0.957 | 0.908 | 1 |
| SLC1A2, OLAH, RETN, MMP8 | | | | | | | | | |
| IFI27, SIGLEC1, HERC6, SLC1A2, | 0.880 | 0.870 | 0.890 | 0.927 | 0.873 | 0.982 | 0.943 | 0.875 | 1 |
| IL1R2, OLAH, FAM20A, RETN | | | | | | | | | |
| IFIT1, SIGLEC1, ISG15, HERC6, | 0.880 | 0.870 | 0.889 | 0.922 | 0.864 | 0.980 | 0.954 | 0.898 | 1 |
| OLAH, FAM20A, RETN, MMP8 | | | | | | | | | |
| RSAD2, IFI27, IFI44L, SIGLEC1, | 0.880 | 0.871 | 0.888 | 0.927 | 0.877 | 0.976 | 0.941 | 0.888 | 0.995 |
| ISG15, IL1R2, OLAH, MMP8 | | | | | | | | | |
| RSAD2, IFI27, IFIT1, IFI44L, | 0.880 | 0.871 | 0.888 | 0.927 | 0.878 | 0.976 | 0.937 | 0.881 | 0.993 |
| HERC6, IL1R2, OLAH, MMP8 | | | | | | | | | |
| RSAD2, IFI27, OAS1, IFIT1, | 0.880 | 0.871 | 0.888 | 0.924 | 0.870 | 0.979 | 0.942 | 0.880 | 1 |
| SIGLEC1, HERC6, SLC1A2, RETN | | | | | | | | | |
| IFI27, OAS1, ISG15, SLC1A2, | 0.880 | 0.869 | 0.890 | 0.929 | 0.876 | 0.982 | 0.922 | 0.842 | 1 |
| OLAH, FAM20A, RETN, MMP8 | | | | | | | | | |
| RSAD2, OAS1, SIGLEC1, ISG15, | 0.880 | 0.870 | 0.889 | 0.918 | 0.861 | 0.975 | 0.961 | 0.902 | 1 |
| HERC6, IL1R2, FAM20A, MMP8 | | | | | | | | | |
| IFI27, IFIT1, IFI44L, SIGLEC1, | 0.880 | 0.871 | 0.888 | 0.930 | 0.881 | 0.980 | 0.942 | 0.884 | 1 |
| ISG15, SLC1A2, OLAH, MMP8 | | | | | | | | | |
| IFI27, OAS1, IFIT1, HERC6, | 0.880 | 0.870 | 0.889 | 0.930 | 0.883 | 0.976 | 0.927 | 0.842 | 1 |
| SLC1A2, IL1R2, FAM20A, MMP8 | | | | | | | | | |
| RSAD2, IFI27, SIGLEC1, ISG15, | 0.880 | 0.871 | 0.888 | 0.924 | 0.871 | 0.977 | 0.961 | 0.920 | 1 |
| HERC6, IL1R2, RETN, MMP8 | | | | | | | | | |
| IFI27, OAS1, IFIT1, ISG15, HERC6, | 0.880 | 0.871 | 0.888 | 0.923 | 0.872 | 0.973 | 0.939 | 0.882 | 0.996 |
| IL1R2, OLAH, MMP8 | | | | | | | | | |
| RSAD2, IFI27, OAS1, IFIT1, ISG15, | 0.880 | 0.871 | 0.888 | 0.923 | 0.869 | 0.976 | 0.942 | 0.881 | 1 |
| HERC6, IL1R2, RETN | | | | | | | | | |
| IFIT1, IFI44L, SIGLEC1, ISG15, | 0.880 | 0.869 | 0.890 | 0.917 | 0.858 | 0.976 | 0.951 | 0.895 | 1 |
| HERC6, OLAH, FAM20A, MMP8 | | | | | | | | | |
| RSAD2, IFI27, OAS1, IFI44L, | 0.880 | 0.870 | 0.889 | 0.930 | 0.876 | 0.983 | 0.920 | 0.837 | 1 |
| SLC1A2, IL1R2, OLAH, FAM20A | | | | | | | | | |
| IFI27, IFI44L, ISG15, SLC1A2, | 0.880 | 0.870 | 0.889 | 0.931 | 0.883 | 0.979 | 0.923 | 0.835 | 1 |
| IL1R2, FAM20A, RETN, MMP8 | | | | | | | | | |
| RSAD2, IFI27, OAS1, IFIT1, | 0.880 | 0.871 | 0.888 | 0.925 | 0.875 | 0.975 | 0.940 | 0.884 | 0.996 |
| IFI44L, HERC6, IL1R2, OLAH | | | | | | | | | |
| IFI27, IFIT1, SIGLEC1, ISG15, | 0.880 | 0.870 | 0.889 | 0.926 | 0.870 | 0.981 | 0.942 | 0.865 | 1 |
| SLC1A2, FAM20A, RETN, MMP8 | | | | | | | | | |
| RSAD2, IFI27, IFIT1, IFI44L, | 0.879 | 0.871 | 0.888 | 0.924 | 0.874 | 0.975 | 0.949 | 0.900 | 0.998 |
| SIGLEC1, IL1R2, OLAH, MMP8 | | | | | | | | | |
| IFI27, OAS1, IFIT1, IFI44L, | 0.879 | 0.871 | 0.888 | 0.924 | 0.874 | 0.974 | 0.941 | 0.888 | 0.995 |
| SIGLEC1, ISG15, IL1R2, OLAH | | | | | | | | | |
| RSAD2, IFI27, IFI44L, SIGLEC1, | 0.879 | 0.870 | 0.889 | 0.924 | 0.869 | 0.978 | 0.957 | 0.906 | 1 |
| IL1R2, OLAH, RETN, MMP8 | | | | | | | | | |
| OAS1, IFIT1, IFI44L, SIGLEC1, | 0.879 | 0.870 | 0.889 | 0.919 | 0.863 | 0.976 | 0.955 | 0.900 | 1 |
| ISG15, HERC6, IL1R2, FAM20A | | | | | | | | | |
| IFI27, OAS1, IFIT1, SIGLEC1, | 0.879 | 0.870 | 0.889 | 0.930 | 0.882 | 0.979 | 0.936 | 0.860 | 1 |
| HERC6, SLC1A2, FAM20A, MMP8 | | | | | | | | | |
| IFI27, OAS1, IFIT1, SLC1A2, | 0.879 | 0.870 | 0.889 | 0.927 | 0.874 | 0.981 | 0.922 | 0.840 | 1 |
| IL1R2, OLAH, FAM20A, RETN | | | | | | | | | |
| IFI27, IFIT1, IFI44L, SIGLEC1, | 0.879 | 0.871 | 0.888 | 0.926 | 0.874 | 0.978 | 0.938 | 0.860 | 1 |
| HERC6, SLC1A2, FAM20A, MMP8 | | | | | | | | | |
| RSAD2, OAS1, IFIT1, SIGLEC1, | 0.879 | 0.870 | 0.888 | 0.919 | 0.862 | 0.977 | 0.967 | 0.914 | 1 |
| ISG15, IL1R2, FAM20A, MMP8 | | | | | | | | | |
| RSAD2, IFI27, OAS1, IFI44L, | 0.879 | 0.869 | 0.890 | 0.929 | 0.877 | 0.982 | 0.935 | 0.856 | 1 |
| IL1R2, FAM20A, RETN, MMP8 | | | | | | | | | |
| IFI27, OAS1, ISG15, HERC6, | 0.879 | 0.870 | 0.889 | 0.929 | 0.879 | 0.978 | 0.938 | 0.874 | 1 |
| SLC1A2, OLAH, RETN, MMP8 | | | | | | | | | |
| IFIT1, IFI44L, SIGLEC1, HERC6, | 0.879 | 0.870 | 0.889 | 0.930 | 0.885 | 0.975 | 0.961 | 0.898 | 1 |
| SLC1A2, IL1R2, FAM20A, MMP8 | | | | | | | | | |
| RSAD2, IFI27, OAS1, IFIT1, ISG15, | 0.879 | 0.871 | 0.888 | 0.924 | 0.874 | 0.974 | 0.939 | 0.882 | 0.996 |
| HERC6, IL1R2, OLAH | | | | | | | | | |
| OAS1, IFIT1, IFI44L, SIGLEC1, | 0.879 | 0.869 | 0.889 | 0.917 | 0.858 | 0.976 | 0.948 | 0.890 | 1 |
| HERC6, OLAH, FAM20A, MMP8 | | | | | | | | | |
| OAS1, IFIT1, SIGLEC1, ISG15, | 0.879 | 0.870 | 0.889 | 0.919 | 0.862 | 0.977 | 0.953 | 0.898 | 1 |
| HERC6, IL1R2, OLAH, FAM20A | | | | | | | | | |
| IFI27, IFI44L, SIGLEC1, ISG15, | 0.879 | 0.870 | 0.888 | 0.930 | 0.880 | 0.981 | 0.952 | 0.897 | 1 |
| SLC1A2, OLAH, RETN, MMP8 | | | | | | | | | |
| IFI27, IFIT1, SIGLEC1, HERC6, | 0.879 | 0.870 | 0.889 | 0.930 | 0.879 | 0.980 | 0.955 | 0.906 | 1 |
| SLC1A2, OLAH, RETN, MMP8 | | | | | | | | | |
| IFI27, OAS1, IFIT1, SIGLEC1, | 0.879 | 0.870 | 0.888 | 0.929 | 0.878 | 0.979 | 0.942 | 0.885 | 1.000 |
| ISG15, SLC1A2, OLAH, MMP8 | | | | | | | | | |
| RSAD2, IFI27, HERC6, SLC1A2, | 0.879 | 0.870 | 0.889 | 0.928 | 0.877 | 0.979 | 0.941 | 0.881 | 1 |
| IL1R2, OLAH, RETN, MMP8 | | | | | | | | | |
| RSAD2, IFI27, IFIT1, IFI44L, | 0.879 | 0.870 | 0.888 | 0.923 | 0.869 | 0.978 | 0.939 | 0.882 | 0.997 |
| HERC6, IL1R2, OLAH, RETN | | | | | | | | | |
| RSAD2, IFI27, OAS1, IFI44L, | 0.879 | 0.871 | 0.888 | 0.924 | 0.874 | 0.975 | 0.945 | 0.892 | 0.997 |
| SIGLEC1, ISG15, IL1R2, OLAH | | | | | | | | | |
| IFI27, IFIT1, SIGLEC1, ISG15, | 0.879 | 0.870 | 0.889 | 0.931 | 0.881 | 0.980 | 0.940 | 0.883 | 0.997 |
| HERC6, SLC1A2, OLAH, MMP8 | | | | | | | | | |
| IFI27, OAS1, IFIT1, IFI44L, | 0.879 | 0.870 | 0.888 | 0.923 | 0.870 | 0.976 | 0.941 | 0.881 | 1 |
| HERC6, IL1R2, RETN, MMP8 | | | | | | | | | |
| IFI27, IFI44L, SIGLEC1, ISG15, | 0.879 | 0.871 | 0.888 | 0.928 | 0.878 | 0.979 | 0.948 | 0.891 | 1 |
| SLC1A2, IL1R2, OLAH, RETN | | | | | | | | | |
| RSAD2, IFI27, IFIT1, IFI44L, | 0.879 | 0.871 | 0.888 | 0.925 | 0.873 | 0.978 | 0.943 | 0.890 | 0.997 |
| SIGLEC1, ISG15, HERC6, OLAH | | | | | | | | | |
| IFI27, OAS1, IFIT1, IFI44L, ISG15, | 0.879 | 0.870 | 0.888 | 0.925 | 0.873 | 0.977 | 0.936 | 0.877 | 0.994 |
| HERC6, OLAH, MMP8 | | | | | | | | | |
| RSAD2, IFI27, OAS1, IFI44L, | 0.879 | 0.871 | 0.887 | 0.924 | 0.877 | 0.971 | 0.941 | 0.889 | 0.993 |
| SIGLEC1, HERC6, IL1R2, MMP8 | | | | | | | | | |
| RSAD2, IFI27, IFIT1, HERC6, | 0.879 | 0.870 | 0.889 | 0.929 | 0.878 | 0.980 | 0.942 | 0.883 | 1 |
| SLC1A2, OLAH, RETN, MMP8 | | | | | | | | | |
| RSAD2, IFI27, OAS1, SIGLEC1, | 0.879 | 0.869 | 0.889 | 0.922 | 0.867 | 0.978 | 0.962 | 0.913 | 1 |
| IL1R2, OLAH, RETN, MMP8 | | | | | | | | | |
| OAS1, IFIT1, IFI44L, SIGLEC1, | 0.879 | 0.870 | 0.888 | 0.923 | 0.867 | 0.979 | 0.968 | 0.918 | 1 |
| IL1R2, FAM20A, RETN, MMP8 | | | | | | | | | |
| IFI27, OAS1, SIGLEC1, ISG15, | 0.879 | 0.870 | 0.889 | 0.923 | 0.868 | 0.978 | 0.960 | 0.911 | 1 |
| IL1R2, OLAH, RETN, MMP8 | | | | | | | | | |
| IFI27, IFIT1, IFI44L, SIGLEC1, | 0.879 | 0.870 | 0.888 | 0.920 | 0.862 | 0.978 | 0.952 | 0.896 | 1 |
| ISG15, OLAH, RETN, MMP8 | | | | | | | | | |
| RSAD2, IFI27, HERC6, SLC1A2, | 0.879 | 0.870 | 0.888 | 0.934 | 0.886 | 0.981 | 0.929 | 0.844 | 1 |
| IL1R2, FAM20A, RETN, MMP8 | | | | | | | | | |
| RSAD2, IFI27, OAS1, IFIT1, ISG15, | 0.879 | 0.870 | 0.888 | 0.923 | 0.871 | 0.976 | 0.936 | 0.877 | 0.995 |
| HERC6, OLAH, MMP8 | | | | | | | | | |
| RSAD2, IFI27, OAS1, SIGLEC1, | 0.879 | 0.870 | 0.889 | 0.928 | 0.880 | 0.977 | 0.946 | 0.889 | 1 |
| HERC6, SLC1A2, IL1R2, OLAH | | | | | | | | | |
| RSAD2, IFI27, SIGLEC1, HERC6, | 0.879 | 0.870 | 0.889 | 0.928 | 0.879 | 0.977 | 0.945 | 0.891 | 0.998 |
| SLC1A2, IL1R2, OLAH, MMP8 | | | | | | | | | |
| IFIT1, IFI44L, SIGLEC1, ISG15, | 0.879 | 0.870 | 0.888 | 0.919 | 0.859 | 0.979 | 0.947 | 0.880 | 1 |
| HERC6, FAM20A, RETN, MMP8 | | | | | | | | | |
| RSAD2, IFI27, OAS1, SIGLEC1, | 0.879 | 0.870 | 0.888 | 0.930 | 0.880 | 0.980 | 0.939 | 0.880 | 0.999 |
| ISG15, HERC6, SLC1A2, OLAH | | | | | | | | | |
| IFI27, IFI44L, HERC6, SLC1A2, | 0.879 | 0.869 | 0.889 | 0.929 | 0.877 | 0.982 | 0.927 | 0.848 | 1 |
| IL1R2, OLAH, FAM20A, RETN | | | | | | | | | |
| RSAD2, SIGLEC1, ISG15, HERC6, | 0.879 | 0.870 | 0.889 | 0.919 | 0.863 | 0.975 | 0.962 | 0.905 | 1 |
| IL1R2, OLAH, FAM20A, MMP8 | | | | | | | | | |
| IFI27, OAS1, SIGLEC1, SLC1A2, | 0.879 | 0.870 | 0.889 | 0.928 | 0.878 | 0.978 | 0.955 | 0.900 | 1 |
| IL1R2, OLAH, RETN, MMP8 | | | | | | | | | |
| RSAD2, OAS1, IFIT1, SIGLEC1, | 0.879 | 0.869 | 0.889 | 0.917 | 0.858 | 0.975 | 0.953 | 0.897 | 1 |
| HERC6, OLAH, FAM20A, MMP8 | | | | | | | | | |
| RSAD2, OAS1, IFIT1, IFI44L, | 0.879 | 0.870 | 0.888 | 0.919 | 0.861 | 0.976 | 0.953 | 0.898 | 1 |
| SIGLEC1, HERC6, IL1R2, FAM20A | | | | | | | | | |
| RSAD2, IFI27, OAS1, IFIT1, | 0.879 | 0.871 | 0.888 | 0.922 | 0.869 | 0.976 | 0.938 | 0.874 | 1 |
| IFI44L, HERC6, IL1R2, RETN | | | | | | | | | |
| IFI27, IFIT1, SIGLEC1, ISG15, | 0.879 | 0.870 | 0.888 | 0.929 | 0.879 | 0.978 | 0.940 | 0.883 | 0.998 |
| HERC6, SLC1A2, IL1R2, OLAH | | | | | | | | | |
| RSAD2, OAS1, IFIT1, SIGLEC1, | 0.879 | 0.870 | 0.888 | 0.919 | 0.861 | 0.976 | 0.953 | 0.898 | 1 |
| ISG15, HERC6, IL1R2, FAM20A | | | | | | | | | |
| RSAD2, IFI27, IFIT1, ISG15, | 0.879 | 0.870 | 0.888 | 0.926 | 0.870 | 0.981 | 0.922 | 0.843 | 1 |
| SLC1A2, OLAH, FAM20A, RETN | | | | | | | | | |
| IFI27, IFIT1, IFI44L, SIGLEC1, | 0.879 | 0.871 | 0.887 | 0.921 | 0.867 | 0.975 | 0.950 | 0.897 | 1 |
| ISG15, IL1R2, OLAH, RETN | | | | | | | | | |
| RSAD2, IFI27, IFIT1, SIGLEC1, | 0.879 | 0.870 | 0.888 | 0.923 | 0.868 | 0.978 | 0.961 | 0.911 | 1 |
| IL1R2, OLAH, RETN, MMP8 | | | | | | | | | |
| RSAD2, IFI27, IFIT1, IFI44L, | 0.879 | 0.871 | 0.887 | 0.917 | 0.859 | 0.975 | 0.941 | 0.887 | 0.996 |
| SIGLEC1, ISG15, HERC6, RETN | | | | | | | | | |
| IFI27, OAS1, IFIT1, SIGLEC1, | 0.879 | 0.870 | 0.888 | 0.926 | 0.874 | 0.978 | 0.946 | 0.888 | 1 |
| HERC6, SLC1A2, RETN, MMP8 | | | | | | | | | |
| IFI27, IFIT1, IFI44L, ISG15, | 0.879 | 0.870 | 0.888 | 0.922 | 0.868 | 0.977 | 0.939 | 0.882 | 0.996 |
| HERC6, IL1R2, OLAH, RETN | | | | | | | | | |
| RSAD2, IFI27, OAS1, IFIT1, | 0.879 | 0.870 | 0.888 | 0.923 | 0.869 | 0.977 | 0.928 | 0.848 | 1 |
| IFI44L, ISG15, IL1R2, FAM20A | | | | | | | | | |
| IFI27, OAS1, IFIT1, IFI44L, | 0.879 | 0.870 | 0.888 | 0.920 | 0.865 | 0.975 | 0.953 | 0.900 | 1 |
| SIGLEC1, IL1R2, OLAH, RETN | | | | | | | | | |
| IFI27, OAS1, IFIT1, IFI44L, | 0.879 | 0.869 | 0.889 | 0.915 | 0.850 | 0.980 | 0.943 | 0.873 | 1 |
| SIGLEC1, ISG15, FAM20A, MMP8 | | | | | | | | | |
| RSAD2, IFI27, OAS1, IFIT1, | 0.879 | 0.870 | 0.888 | 0.928 | 0.878 | 0.978 | 0.942 | 0.884 | 1 |
| SIGLEC1, HERC6, SLC1A2, OLAH | | | | | | | | | |
| RSAD2, IFI27, IFIT1, ISG15, | 0.879 | 0.869 | 0.889 | 0.927 | 0.871 | 0.982 | 0.934 | 0.854 | 1 |
| HERC6, FAM20A, RETN, MMP8 | | | | | | | | | |
| IFI27, IFIT1, IFI44L, SIGLEC1, | 0.879 | 0.870 | 0.888 | 0.927 | 0.876 | 0.978 | 0.941 | 0.864 | 1 |
| ISG15, HERC6, SLC1A2, FAM20A | | | | | | | | | |
| IFIT1, IFI44L, SIGLEC1, IL1R2, | 0.879 | 0.870 | 0.888 | 0.919 | 0.862 | 0.977 | 0.967 | 0.917 | 1 |
| OLAH, FAM20A, RETN, MMP8 | | | | | | | | | |
| IFI27, OAS1, IFIT1, SIGLEC1, | 0.879 | 0.870 | 0.888 | 0.928 | 0.881 | 0.975 | 0.945 | 0.888 | 1 |
| HERC6, SLC1A2, IL1R2, RETN | | | | | | | | | |
| RSAD2, IFI27, IFIT1, SIGLEC1, | 0.879 | 0.870 | 0.888 | 0.924 | 0.872 | 0.975 | 0.937 | 0.880 | 0.994 |
| ISG15, HERC6, OLAH, MMP8 | | | | | | | | | |
| IFI27, OAS1, IFIT1, SIGLEC1, | 0.879 | 0.870 | 0.888 | 0.927 | 0.876 | 0.978 | 0.942 | 0.884 | 1 |
| ISG15, HERC6, SLC1A2, RETN | | | | | | | | | |
| RSAD2, IFI44L, SIGLEC1, HERC6, | 0.879 | 0.869 | 0.888 | 0.921 | 0.866 | 0.976 | 0.961 | 0.903 | 1 |
| IL1R2, OLAH, FAM20A, MMP8 | | | | | | | | | |
| RSAD2, IFI27, IFI44L, ISG15, | 0.879 | 0.869 | 0.889 | 0.927 | 0.875 | 0.980 | 0.932 | 0.851 | 1 |
| IL1R2, FAM20A, RETN, MMP8 | | | | | | | | | |
| RSAD2, IFI27, OAS1, SLC1A2, | 0.879 | 0.869 | 0.888 | 0.927 | 0.875 | 0.980 | 0.918 | 0.834 | 1 |
| IL1R2, OLAH, FAM20A, RETN | | | | | | | | | |
| RSAD2, IFIT1, IFI44L, SIGLEC1, | 0.879 | 0.870 | 0.888 | 0.918 | 0.860 | 0.976 | 0.952 | 0.895 | 1 |
| HERC6, IL1R2, OLAH, FAM20A | | | | | | | | | |
| OAS1, SIGLEC1, ISG15, HERC6, | 0.879 | 0.869 | 0.888 | 0.918 | 0.861 | 0.975 | 0.963 | 0.908 | 1 |
| IL1R2, OLAH, FAM20A, MMP8 | | | | | | | | | |
| RSAD2, IFI27, IFIT1, SLC1A2, | 0.879 | 0.870 | 0.888 | 0.927 | 0.872 | 0.981 | 0.921 | 0.839 | 1 |
| IL1R2, OLAH, FAM20A, RETN | | | | | | | | | |
| RSAD2, IFI27, IFIT1, SIGLEC1, | 0.879 | 0.869 | 0.888 | 0.926 | 0.871 | 0.982 | 0.946 | 0.873 | 1 |
| SLC1A2, FAM20A, RETN, MMP8 | | | | | | | | | |
| IFI27, IFIT1, SIGLEC1, ISG15, | 0.879 | 0.870 | 0.888 | 0.929 | 0.879 | 0.980 | 0.941 | 0.881 | 1 |
| SLC1A2, IL1R2, OLAH, RETN | | | | | | | | | |
| RSAD2, OAS1, IFIT1, SIGLEC1, | 0.879 | 0.870 | 0.888 | 0.918 | 0.860 | 0.976 | 0.955 | 0.901 | 1 |
| HERC6, IL1R2, OLAH, FAM20A | | | | | | | | | |
| IFI27, OAS1, IFIT1, HERC6, | 0.879 | 0.869 | 0.888 | 0.927 | 0.876 | 0.978 | 0.934 | 0.867 | 1 |
| SLC1A2, IL1R2, OLAH, RETN | | | | | | | | | |
| OAS1, IFI44L, SIGLEC1, HERC6, | 0.879 | 0.869 | 0.888 | 0.920 | 0.863 | 0.976 | 0.962 | 0.906 | 1 |
| IL1R2, OLAH, FAM20A, MMP8 | | | | | | | | | |
| RSAD2, IFI27, IFIT1, IFI44L, | 0.879 | 0.869 | 0.889 | 0.925 | 0.868 | 0.982 | 0.934 | 0.853 | 1 |
| HERC6, FAM20A, RETN, MMP8 | | | | | | | | | |
| IFI27, OAS1, IFI44L, SIGLEC1, | 0.879 | 0.870 | 0.887 | 0.921 | 0.866 | 0.976 | 0.947 | 0.894 | 0.999 |
| ISG15, IL1R2, OLAH, RETN | | | | | | | | | |
| IFI27, OAS1, SIGLEC1, ISG15, | 0.879 | 0.870 | 0.888 | 0.930 | 0.882 | 0.979 | 0.940 | 0.882 | 0.999 |
| SLC1A2, IL1R2, OLAH, MMP8 | | | | | | | | | |
| RSAD2, IFI27, IFIT1, IFI44L, | 0.879 | 0.870 | 0.888 | 0.927 | 0.875 | 0.978 | 0.937 | 0.860 | 1 |
| SIGLEC1, HERC6, SLC1A2, FAM20A | | | | | | | | | |
| IFI27, OAS1, IFIT1, SIGLEC1, | 0.879 | 0.870 | 0.887 | 0.919 | 0.864 | 0.975 | 0.947 | 0.890 | 1 |
| ISG15, IL1R2, OLAH, RETN | | | | | | | | | |
| RSAD2, IFI27, IFI44L, ISG15, | 0.879 | 0.868 | 0.889 | 0.925 | 0.868 | 0.982 | 0.937 | 0.858 | 1 |
| HERC6, FAM20A, RETN, MMP8 | | | | | | | | | |
| RSAD2, IFI27, OAS1, IFI44L, | 0.879 | 0.870 | 0.887 | 0.922 | 0.868 | 0.975 | 0.938 | 0.873 | 1 |
| ISG15, HERC6, IL1R2, RETN | | | | | | | | | |
| IFI27, OAS1, IFIT1, ISG15, HERC6, | 0.879 | 0.870 | 0.888 | 0.924 | 0.871 | 0.977 | 0.945 | 0.886 | 1 |
| IL1R2, RETN, MMP8 | | | | | | | | | |
| RSAD2, IFI27, OAS1, IFI44L, | 0.879 | 0.868 | 0.889 | 0.925 | 0.867 | 0.983 | 0.935 | 0.855 | 1 |
| HERC6, FAM20A, RETN, MMP8 | | | | | | | | | |
| IFIT1, IFI44L, SIGLEC1, ISG15, | 0.879 | 0.869 | 0.888 | 0.918 | 0.858 | 0.978 | 0.945 | 0.886 | 1 |
| HERC6, OLAH, FAM20A, RETN | | | | | | | | | |
| RSAD2, IFI27, OAS1, ISG15, | 0.879 | 0.869 | 0.888 | 0.927 | 0.874 | 0.981 | 0.917 | 0.833 | 1 |
| SLC1A2, OLAH, FAM20A, RETN | | | | | | | | | |
| RSAD2, IFI27, OAS1, IFIT1, | 0.879 | 0.869 | 0.888 | 0.920 | 0.861 | 0.978 | 0.962 | 0.911 | 1 |
| SIGLEC1, OLAH, RETN, MMP8 | | | | | | | | | |
| RSAD2, IFI27, SIGLEC1, ISG15, | 0.879 | 0.869 | 0.888 | 0.931 | 0.881 | 0.980 | 0.939 | 0.881 | 0.998 |
| HERC6, SLC1A2, OLAH, MMP8 | | | | | | | | | |
| OAS1, IFI44L, SIGLEC1, ISG15, | 0.879 | 0.869 | 0.888 | 0.917 | 0.859 | 0.974 | 0.960 | 0.900 | 1 |
| HERC6, IL1R2, FAM20A, MMP8 | | | | | | | | | |
| OAS1, IFIT1, IFI44L, SIGLEC1, | 0.879 | 0.869 | 0.888 | 0.919 | 0.861 | 0.976 | 0.966 | 0.913 | 1 |
| ISG15, IL1R2, FAM20A, MMP8 | | | | | | | | | |
| IFI27, SIGLEC1, ISG15, HERC6, | 0.879 | 0.869 | 0.889 | 0.932 | 0.884 | 0.980 | 0.947 | 0.877 | 1 |
| SLC1A2, IL1R2, FAM20A, RETN | | | | | | | | | |
| RSAD2, IFI27, IFIT1, IFI44L, | 0.879 | 0.870 | 0.888 | 0.921 | 0.863 | 0.979 | 0.955 | 0.900 | 1 |
| SIGLEC1, OLAH, RETN, MMP8 | | | | | | | | | |
| IFI27, OAS1, IFI44L, HERC6, | 0.879 | 0.868 | 0.889 | 0.930 | 0.884 | 0.976 | 0.923 | 0.834 | 1 |
| SLC1A2, IL1R2, FAM20A, MMP8 | | | | | | | | | |
| RSAD2, IFI27, OAS1, IFIT1, | 0.879 | 0.870 | 0.887 | 0.921 | 0.869 | 0.973 | 0.951 | 0.902 | 1 |
| IFI44L, SIGLEC1, IL1R2, OLAH | | | | | | | | | |
| RSAD2, OAS1, SIGLEC1, ISG15, | 0.879 | 0.869 | 0.888 | 0.918 | 0.861 | 0.976 | 0.963 | 0.906 | 1 |
| IL1R2, OLAH, FAM20A, MMP8 | | | | | | | | | |
| RSAD2, IFIT1, SIGLEC1, ISG15, | 0.879 | 0.869 | 0.888 | 0.918 | 0.859 | 0.977 | 0.952 | 0.897 | 1 |
| HERC6, IL1R2, OLAH, FAM20A | | | | | | | | | |
| RSAD2, IFI27, OAS1, ISG15, | 0.879 | 0.870 | 0.887 | 0.923 | 0.869 | 0.977 | 0.942 | 0.880 | 1 |
| HERC6, IL1R2, RETN, MMP8 | | | | | | | | | |
| RSAD2, IFI27, OAS1, SIGLEC1, | 0.879 | 0.869 | 0.889 | 0.925 | 0.872 | 0.979 | 0.937 | 0.863 | 1 |
| HERC6, SLC1A2, FAM20A, RETN | | | | | | | | | |
| IFI27, SIGLEC1, ISG15, HERC6, | 0.879 | 0.870 | 0.888 | 0.929 | 0.880 | 0.978 | 0.942 | 0.888 | 0.997 |
| SLC1A2, IL1R2, OLAH, MMP8 | | | | | | | | | |
| RSAD2, IFI27, OAS1, IFI44L, | 0.879 | 0.869 | 0.888 | 0.921 | 0.863 | 0.978 | 0.960 | 0.908 | 1 |
| SIGLEC1, OLAH, RETN, MMP8 | | | | | | | | | |
| IFI27, IFIT1, IFI44L, HERC6, | 0.879 | 0.869 | 0.888 | 0.930 | 0.884 | 0.977 | 0.929 | 0.845 | 1 |
| SLC1A2, IL1R2, FAM20A, MMP8 | | | | | | | | | |
| IFI27, OAS1, IFIT1, IFI44L, | 0.879 | 0.870 | 0.887 | 0.926 | 0.874 | 0.977 | 0.941 | 0.882 | 1 |
| SIGLEC1, HERC6, SLC1A2, RETN | | | | | | | | | |
| IFI44L, SIGLEC1, ISG15, SLC1A2, | 0.878 | 0.870 | 0.887 | 0.923 | 0.875 | 0.972 | 0.959 | 0.895 | 1 |
| IL1R2, OLAH, FAM20A, MMP8 | | | | | | | | | |
| IFI27, OAS1, ISG15, HERC6, | 0.878 | 0.869 | 0.888 | 0.933 | 0.885 | 0.980 | 0.923 | 0.836 | 1 |
| SLC1A2, IL1R2, FAM20A, RETN | | | | | | | | | |
| IFI27, OAS1, IFIT1, ISG15, HERC6, | 0.878 | 0.869 | 0.888 | 0.928 | 0.878 | 0.978 | 0.935 | 0.869 | 1 |
| SLC1A2, OLAH, RETN | | | | | | | | | |
| OAS1, IFIT1, SIGLEC1, SLC1A2, | 0.878 | 0.869 | 0.888 | 0.926 | 0.878 | 0.975 | 0.962 | 0.906 | 1 |
| IL1R2, OLAH, FAM20A, MMP8 | | | | | | | | | |
| IFI27, OAS1, IFI44L, ISG15, | 0.878 | 0.868 | 0.889 | 0.930 | 0.884 | 0.976 | 0.925 | 0.837 | 1 |
| HERC6, SLC1A2, IL1R2, FAM20A | | | | | | | | | |
| RSAD2, IFI27, OAS1, HERC6, | 0.878 | 0.869 | 0.888 | 0.928 | 0.876 | 0.979 | 0.932 | 0.860 | 1 |
| SLC1A2, IL1R2, OLAH, RETN | | | | | | | | | |
| IFI27, OAS1, ISG15, HERC6, | 0.878 | 0.869 | 0.888 | 0.930 | 0.880 | 0.979 | 0.930 | 0.862 | 0.999 |
| SLC1A2, IL1R2, OLAH, RETN | | | | | | | | | |
| IFI27, OAS1, IFIT1, IFI44L, ISG15, | 0.878 | 0.870 | 0.887 | 0.924 | 0.873 | 0.976 | 0.938 | 0.877 | 1.000 |
| HERC6, IL1R2, RETN | | | | | | | | | |
| RSAD2, IFI27, SIGLEC1, ISG15, | 0.878 | 0.870 | 0.887 | 0.929 | 0.880 | 0.978 | 0.942 | 0.887 | 0.998 |
| SLC1A2, IL1R2, OLAH, MMP8 | | | | | | | | | |
| RSAD2, IFI27, OAS1, SIGLEC1, | 0.878 | 0.870 | 0.887 | 0.926 | 0.877 | 0.975 | 0.948 | 0.888 | 1 |
| HERC6, SLC1A2, IL1R2, RETN | | | | | | | | | |
| IFI27, IFIT1, IFI44L, SLC1A2, | 0.878 | 0.869 | 0.888 | 0.932 | 0.883 | 0.981 | 0.932 | 0.849 | 1 |
| IL1R2, FAM20A, RETN, MMP8 | | | | | | | | | |
| RSAD2, IFI27, OAS1, IFIT1, | 0.878 | 0.869 | 0.887 | 0.924 | 0.873 | 0.976 | 0.934 | 0.874 | 0.994 |
| IFI44L, ISG15, HERC6, OLAH | | | | | | | | | |
| IFI27, OAS1, IFIT1, SIGLEC1, | 0.878 | 0.869 | 0.888 | 0.924 | 0.868 | 0.980 | 0.938 | 0.862 | 1 |
| ISG15, SLC1A2, FAM20A, RETN | | | | | | | | | |
| IFI27, IFIT1, SIGLEC1, ISG15, | 0.878 | 0.869 | 0.888 | 0.928 | 0.877 | 0.979 | 0.941 | 0.884 | 0.999 |
| HERC6, SLC1A2, OLAH, RETN | | | | | | | | | |
| IFI27, IFIT1, IFI44L, SIGLEC1, | 0.878 | 0.870 | 0.887 | 0.925 | 0.873 | 0.977 | 0.959 | 0.916 | 1 |
| ISG15, IL1R2, RETN, MMP8 | | | | | | | | | |
| IFI44L, SIGLEC1, ISG15, HERC6, | 0.878 | 0.869 | 0.888 | 0.917 | 0.858 | 0.975 | 0.958 | 0.894 | 1 |
| IL1R2, FAM20A, RETN, MMP8 | | | | | | | | | |
| IFI27, OAS1, IFIT1, IFI44L, | 0.878 | 0.870 | 0.887 | 0.924 | 0.872 | 0.976 | 0.958 | 0.913 | 1 |
| SIGLEC1, IL1R2, RETN, MMP8 | | | | | | | | | |
| IFI27, OAS1, IFI44L, HERC6, | 0.878 | 0.869 | 0.888 | 0.930 | 0.881 | 0.980 | 0.938 | 0.872 | 1 |
| SLC1A2, OLAH, RETN, MMP8 | | | | | | | | | |
| IFI27, OAS1, IFI44L, SIGLEC1, | 0.878 | 0.869 | 0.888 | 0.922 | 0.866 | 0.979 | 0.957 | 0.905 | 1 |
| ISG15, OLAH, RETN, MMP8 | | | | | | | | | |
| IFI27, OAS1, IFI44L, SLC1A2, | 0.878 | 0.868 | 0.889 | 0.929 | 0.874 | 0.984 | 0.928 | 0.851 | 1 |
| OLAH, FAM20A, RETN, MMP8 | | | | | | | | | |
| IFI27, OAS1, IFIT1, IFI44L, | 0.878 | 0.870 | 0.887 | 0.919 | 0.862 | 0.977 | 0.948 | 0.891 | 1 |
| SIGLEC1, ISG15, OLAH, RETN | | | | | | | | | |
| RSAD2, IFI27, OAS1, IFI44L, | 0.878 | 0.869 | 0.887 | 0.921 | 0.866 | 0.976 | 0.937 | 0.872 | 1 |
| HERC6, IL1R2, RETN, MMP8 | | | | | | | | | |
| IFI27, SIGLEC1, ISG15, SLC1A2, | 0.878 | 0.869 | 0.888 | 0.929 | 0.880 | 0.979 | 0.957 | 0.906 | 1 |
| IL1R2, OLAH, RETN, MMP8 | | | | | | | | | |
| RSAD2, IFI27, IFI44L, HERC6, | 0.878 | 0.869 | 0.888 | 0.930 | 0.884 | 0.976 | 0.927 | 0.840 | 1 |
| SLC1A2, IL1R2, FAM20A, MMP8 | | | | | | | | | |
| IFI27, IFIT1, IFI44L, SIGLEC1, | 0.878 | 0.869 | 0.888 | 0.926 | 0.876 | 0.977 | 0.945 | 0.889 | 1 |
| HERC6, SLC1A2, OLAH, RETN | | | | | | | | | |
| IFI27, OAS1, IFI44L, ISG15, | 0.878 | 0.869 | 0.887 | 0.924 | 0.871 | 0.977 | 0.937 | 0.874 | 1.000 |
| HERC6, IL1R2, RETN, MMP8 | | | | | | | | | |
| RSAD2, IFIT1, SIGLEC1, HERC6, | 0.878 | 0.869 | 0.888 | 0.928 | 0.882 | 0.974 | 0.959 | 0.895 | 1 |
| SLC1A2, IL1R2, FAM20A, MMP8 | | | | | | | | | |
| IFI27, OAS1, IFIT1, HERC6, | 0.878 | 0.869 | 0.888 | 0.928 | 0.879 | 0.976 | 0.935 | 0.870 | 1 |
| SLC1A2, IL1R2, OLAH, MMP8 | | | | | | | | | |
| IFIT1, IFI44L, SIGLEC1, ISG15, | 0.878 | 0.869 | 0.887 | 0.918 | 0.861 | 0.976 | 0.953 | 0.897 | 1 |
| HERC6, IL1R2, FAM20A, RETN | | | | | | | | | |
| IFIT1, SIGLEC1, ISG15, HERC6, | 0.878 | 0.868 | 0.889 | 0.925 | 0.876 | 0.975 | 0.947 | 0.877 | 1 |
| SLC1A2, OLAH, FAM20A, MMP8 | | | | | | | | | |
| RSAD2, IFI27, IFI44L, SIGLEC1, | 0.878 | 0.870 | 0.887 | 0.923 | 0.869 | 0.976 | 0.945 | 0.893 | 0.996 |
| ISG15, HERC6, IL1R2, RETN | | | | | | | | | |
| IFI27, OAS1, IFIT1, SIGLEC1, | 0.878 | 0.869 | 0.887 | 0.930 | 0.881 | 0.979 | 0.941 | 0.883 | 1.000 |
| ISG15, SLC1A2, IL1R2, OLAH | | | | | | | | | |
| IFI27, OAS1, IFIT1, IFI44L, ISG15, | 0.878 | 0.869 | 0.888 | 0.916 | 0.857 | 0.976 | 0.934 | 0.867 | 1 |
| HERC6, RETN, MMP8 | | | | | | | | | |
| RSAD2, IFI27, OAS1, SIGLEC1, | 0.878 | 0.869 | 0.887 | 0.929 | 0.878 | 0.980 | 0.943 | 0.886 | 1 |
| ISG15, SLC1A2, OLAH, MMP8 | | | | | | | | | |
| IFI27, IFIT1, HERC6, SLC1A2, | 0.878 | 0.869 | 0.888 | 0.933 | 0.884 | 0.982 | 0.930 | 0.847 | 1 |
| IL1R2, FAM20A, RETN, MMP8 | | | | | | | | | |
| IFI27, IFI44L, SIGLEC1, ISG15, | 0.878 | 0.869 | 0.887 | 0.927 | 0.875 | 0.978 | 0.943 | 0.887 | 1 |
| HERC6, SLC1A2, OLAH, RETN | | | | | | | | | |
| OAS1, IFIT1, SIGLEC1, ISG15, | 0.878 | 0.869 | 0.887 | 0.922 | 0.865 | 0.979 | 0.967 | 0.914 | 1 |
| IL1R2, FAM20A, RETN, MMP8 | | | | | | | | | |
| IFI27, IFIT1, SIGLEC1, HERC6, | 0.878 | 0.869 | 0.888 | 0.927 | 0.876 | 0.978 | 0.948 | 0.894 | 1 |
| SLC1A2, IL1R2, OLAH, RETN | | | | | | | | | |
| RSAD2, IFI27, SIGLEC1, HERC6, | 0.878 | 0.869 | 0.888 | 0.928 | 0.878 | 0.978 | 0.946 | 0.890 | 1 |
| SLC1A2, IL1R2, OLAH, RETN | | | | | | | | | |
| RSAD2, OAS1, SIGLEC1, HERC6, | 0.878 | 0.869 | 0.888 | 0.921 | 0.865 | 0.977 | 0.959 | 0.898 | 1 |
| IL1R2, FAM20A, RETN, MMP8 | | | | | | | | | |
| RSAD2, IFI27, SIGLEC1, ISG15, | 0.878 | 0.869 | 0.887 | 0.930 | 0.881 | 0.979 | 0.940 | 0.883 | 0.998 |
| HERC6, SLC1A2, IL1R2, OLAH | | | | | | | | | |
| IFI27, OAS1, IFIT1, IFI44L, ISG15, | 0.878 | 0.870 | 0.887 | 0.924 | 0.874 | 0.975 | 0.936 | 0.878 | 0.994 |
| HERC6, IL1R2, OLAH | | | | | | | | | |
| IFI27, IFIT1, IFI44L, ISG15, | 0.878 | 0.870 | 0.887 | 0.925 | 0.875 | 0.975 | 0.937 | 0.880 | 0.993 |
| HERC6, IL1R2, OLAH, MMP8 | | | | | | | | | |
| OAS1, IFIT1, IFI44L, SIGLEC1, | 0.878 | 0.868 | 0.888 | 0.924 | 0.870 | 0.978 | 0.943 | 0.881 | 1 |
| SLC1A2, OLAH, FAM20A, MMP8 | | | | | | | | | |
| IFI27, OAS1, IFI44L, SIGLEC1, | 0.878 | 0.869 | 0.888 | 0.929 | 0.879 | 0.979 | 0.939 | 0.881 | 0.997 |
| ISG15, HERC6, SLC1A2, OLAH | | | | | | | | | |
| OAS1, IFIT1, SIGLEC1, HERC6, | 0.878 | 0.868 | 0.888 | 0.926 | 0.877 | 0.975 | 0.942 | 0.871 | 1 |
| SLC1A2, OLAH, FAM20A, MMP8 | | | | | | | | | |
| IFI27, OAS1, IFI44L, SIGLEC1, | 0.878 | 0.869 | 0.888 | 0.927 | 0.877 | 0.977 | 0.941 | 0.844 | 0.999 |
| HERC6, SLC1A2, OLAH, MMP8 | | | | | | | | | |
| IFI27, OAS1, IFIT1, IFI44L, | 0.878 | 0.869 | 0.887 | 0.920 | 0.863 | 0.976 | 0.939 | 0.882 | 0.996 |
| SIGLEC1, ISG15, OLAH, MMP8 | | | | | | | | | |
| RSAD2, IFI27, OAS1, IFIT1, | 0.878 | 0.869 | 0.888 | 0.927 | 0.876 | 0.978 | 0.932 | 0.860 | 1 |
| HERC6, SLC1A2, OLAH, RETN | | | | | | | | | |
| RSAD2, IFI27, OAS1, IFIT1, | 0.878 | 0.869 | 0.887 | 0.922 | 0.871 | 0.973 | 0.947 | 0.896 | 0.998 |
| SIGLEC1, IL1R2, OLAH, MMP8 | | | | | | | | | |
| RSAD2, IFI27, OAS1, IFI44L, | 0.878 | 0.869 | 0.887 | 0.920 | 0.865 | 0.975 | 0.953 | 0.897 | 1 |
| SIGLEC1, IL1R2, OLAH, RETN | | | | | | | | | |
| RSAD2, IFI27, OAS1, IFIT1, | 0.878 | 0.868 | 0.888 | 0.924 | 0.866 | 0.982 | 0.934 | 0.853 | 1 |
| HERC6, FAM20A, RETN, MMP8 | | | | | | | | | |
| IFI27, IFI44L, SIGLEC1, SLC1A2, | 0.878 | 0.869 | 0.887 | 0.930 | 0.881 | 0.980 | 0.958 | 0.908 | 1 |
| IL1R2, OLAH, RETN, MMP8 | | | | | | | | | |
| IFI27, OAS1, IFIT1, SIGLEC1, | 0.878 | 0.868 | 0.888 | 0.927 | 0.875 | 0.980 | 0.957 | 0.901 | 1 |
| SLC1A2, OLAH, RETN, MMP8 | | | | | | | | | |
| IFI27, IFIT1, SIGLEC1, ISG15, | 0.878 | 0.869 | 0.887 | 0.921 | 0.867 | 0.976 | 0.957 | 0.914 | 0.999 |
| HERC6, IL1R2, RETN, MMP8 | | | | | | | | | |
| RSAD2, IFI27, IFI44L, SLC1A2, | 0.878 | 0.868 | 0.888 | 0.927 | 0.872 | 0.981 | 0.923 | 0.843 | 1 |
| IL1R2, OLAH, FAM20A, RETN | | | | | | | | | |
| RSAD2, IFI27, IFIT1, HERC6, | 0.878 | 0.869 | 0.888 | 0.931 | 0.884 | 0.978 | 0.929 | 0.845 | 1 |
| SLC1A2, IL1R2, FAM20A, MMP8 | | | | | | | | | |
| RSAD2, IFI27, IFI44L, SIGLEC1, | 0.878 | 0.869 | 0.887 | 0.926 | 0.876 | 0.976 | 0.942 | 0.884 | 1 |
| HERC6, SLC1A2, OLAH, RETN | | | | | | | | | |
| IFI27, OAS1, IFIT1, IFI44L, | 0.878 | 0.869 | 0.888 | 0.932 | 0.886 | 0.977 | 0.926 | 0.840 | 1 |
| HERC6, SLC1A2, IL1R2, FAM20A | | | | | | | | | |
| IFI27, OAS1, IFIT1, SIGLEC1, | 0.878 | 0.869 | 0.887 | 0.927 | 0.876 | 0.978 | 0.949 | 0.889 | 1 |
| SLC1A2, IL1R2, OLAH, RETN | | | | | | | | | |
| RSAD2, IFI27, OAS1, IFIT1, | 0.878 | 0.868 | 0.888 | 0.923 | 0.869 | 0.977 | 0.930 | 0.852 | 1 |
| IFI44L, IL1R2, FAM20A, RETN | | | | | | | | | |
| IFI27, IFI44L, SIGLEC1, ISG15, | 0.878 | 0.869 | 0.887 | 0.930 | 0.880 | 0.980 | 0.941 | 0.885 | 0.998 |
| HERC6, SLC1A2, OLAH, MMP8 | | | | | | | | | |
| RSAD2, IFIT1, IFI44L, SIGLEC1, | 0.878 | 0.868 | 0.888 | 0.926 | 0.873 | 0.978 | 0.942 | 0.879 | 1 |
| SLC1A2, OLAH, FAM20A, MMP8 | | | | | | | | | |
| IFI27, OAS1, IFIT1, IFI44L, | 0.878 | 0.869 | 0.888 | 0.927 | 0.877 | 0.977 | 0.942 | 0.885 | 0.999 |
| SIGLEC1, HERC6, SLC1A2, OLAH | | | | | | | | | |
| IFI27, IFIT1, SIGLEC1, ISG15, | 0.878 | 0.869 | 0.888 | 0.929 | 0.877 | 0.980 | 0.954 | 0.900 | 1 |
| SLC1A2, OLAH, RETN, MMP8 | | | | | | | | | |
| IFI27, SIGLEC1, ISG15, HERC6, | 0.878 | 0.868 | 0.888 | 0.927 | 0.875 | 0.980 | 0.953 | 0.900 | 1 |
| SLC1A2, OLAH, RETN, MMP8 | | | | | | | | | |
| RSAD2, IFI27, OAS1, SIGLEC1, | 0.878 | 0.868 | 0.888 | 0.916 | 0.856 | 0.976 | 0.941 | 0.871 | 1 |
| ISG15, HERC6, FAM20A, MMP8 | | | | | | | | | |
| OAS1, IFIT1, IFI44L, SIGLEC1, | 0.878 | 0.869 | 0.887 | 0.919 | 0.859 | 0.978 | 0.947 | 0.883 | 1 |
| HERC6, FAM20A, RETN, MMP8 | | | | | | | | | |
| RSAD2, IFI27, OAS1, IFIT1, | 0.878 | 0.870 | 0.886 | 0.924 | 0.876 | 0.971 | 0.947 | 0.898 | 0.996 |
| IFI44L, SIGLEC1, HERC6, IL1R2 | | | | | | | | | |
| RSAD2, IFI27, OAS1, IFI44L, | 0.878 | 0.870 | 0.887 | 0.919 | 0.862 | 0.975 | 0.942 | 0.887 | 0.997 |
| SIGLEC1, ISG15, OLAH, MMP8 | | | | | | | | | |
| IFI27, OAS1, IFIT1, SIGLEC1, | 0.878 | 0.870 | 0.887 | 0.923 | 0.873 | 0.974 | 0.947 | 0.897 | 0.997 |
| ISG15, IL1R2, OLAH, MMP8 | | | | | | | | | |
| RSAD2, IFI27, IFI44L, SIGLEC1, | 0.878 | 0.870 | 0.887 | 0.922 | 0.867 | 0.977 | 0.951 | 0.900 | 1 |
| ISG15, IL1R2, OLAH, RETN | | | | | | | | | |
| RSAD2, OAS1, IFI44L, SIGLEC1, | 0.878 | 0.868 | 0.888 | 0.919 | 0.862 | 0.976 | 0.962 | 0.906 | 1 |
| IL1R2, OLAH, FAM20A, MMP8 | | | | | | | | | |
| IFIT1, IFI44L, SIGLEC1, ISG15, | 0.878 | 0.869 | 0.887 | 0.922 | 0.866 | 0.979 | 0.968 | 0.918 | 1 |
| IL1R2, FAM20A, RETN, MMP8 | | | | | | | | | |
| IFIT1, SIGLEC1, ISG15, IL1R2, | 0.878 | 0.869 | 0.887 | 0.921 | 0.864 | 0.978 | 0.966 | 0.915 | 1 |
| OLAH, FAM20A, RETN, MMP8 | | | | | | | | | |
| IFI27, IFI44L, HERC6, SLC1A2, | 0.878 | 0.868 | 0.888 | 0.932 | 0.883 | 0.981 | 0.930 | 0.847 | 1 |
| IL1R2, FAM20A, RETN, MMP8 | | | | | | | | | |
| IFI27, IFIT1, IFI44L, SIGLEC1, | 0.878 | 0.869 | 0.887 | 0.928 | 0.877 | 0.979 | 0.947 | 0.889 | 1 |
| ISG15, SLC1A2, OLAH, RETN | | | | | | | | | |
| IFI27, OAS1, ISG15, HERC6, | 0.878 | 0.869 | 0.887 | 0.927 | 0.878 | 0.976 | 0.928 | 0.862 | 0.995 |
| SLC1A2, IL1R2, OLAH, MMP8 | | | | | | | | | |
| RSAD2, IFI27, IFIT1, SIGLEC1, | 0.878 | 0.870 | 0.886 | 0.924 | 0.872 | 0.975 | 0.949 | 0.901 | 0.997 |
| ISG15, HERC6, IL1R2, RETN | | | | | | | | | |
| IFIT1, IFI44L, SIGLEC1, ISG15, | 0.878 | 0.868 | 0.888 | 0.924 | 0.873 | 0.976 | 0.930 | 0.858 | 1 |
| HERC6, SLC1A2, OLAH, FAM20A | | | | | | | | | |
| IFI27, IFIT1, HERC6, SLC1A2, | 0.878 | 0.869 | 0.887 | 0.927 | 0.876 | 0.979 | 0.941 | 0.881 | 1 |
| IL1R2, OLAH, RETN, MMP8 | | | | | | | | | |
| RSAD2, IFI27, ISG15, HERC6, | 0.878 | 0.869 | 0.887 | 0.928 | 0.878 | 0.979 | 0.941 | 0.881 | 1 |
| SLC1A2, OLAH, RETN, MMP8 | | | | | | | | | |
| IFI27, OAS1, SIGLEC1, ISG15, | 0.878 | 0.869 | 0.887 | 0.928 | 0.881 | 0.975 | 0.948 | 0.891 | 1 |
| HERC6, SLC1A2, IL1R2, RETN | | | | | | | | | |
| IFI27, OAS1, IFIT1, SIGLEC1, | 0.878 | 0.869 | 0.887 | 0.927 | 0.878 | 0.976 | 0.949 | 0.894 | 1 |
| SLC1A2, IL1R2, OLAH, MMP8 | | | | | | | | | |
| RSAD2, IFI27, OAS1, ISG15, | 0.878 | 0.868 | 0.888 | 0.929 | 0.877 | 0.981 | 0.935 | 0.856 | 1 |
| IL1R2, FAM20A, RETN, MMP8 | | | | | | | | | |
| RSAD2, IFI27, SIGLEC1, ISG15, | 0.878 | 0.869 | 0.887 | 0.922 | 0.867 | 0.978 | 0.957 | 0.907 | 1 |
| IL1R2, OLAH, RETN, MMP8 | | | | | | | | | |
| RSAD2, IFI27, IFIT1, IFI44L, | 0.878 | 0.868 | 0.888 | 0.924 | 0.871 | 0.977 | 0.930 | 0.852 | 1 |
| ISG15, IL1R2, FAM20A, RETN | | | | | | | | | |
| IFI27, OAS1, IFIT1, IFI44L, IL1R2, | 0.878 | 0.869 | 0.887 | 0.922 | 0.866 | 0.977 | 0.950 | 0.895 | 1 |
| OLAH, RETN, MMP8 | | | | | | | | | |
| IFI27, OAS1, IFI44L, SLC1A2, | 0.878 | 0.868 | 0.888 | 0.929 | 0.875 | 0.983 | 0.921 | 0.839 | 1 |
| IL1R2, OLAH, FAM20A, RETN | | | | | | | | | |
| OAS1, IFIT1, SIGLEC1, ISG15, | 0.878 | 0.868 | 0.888 | 0.917 | 0.858 | 0.975 | 0.953 | 0.899 | 1 |
| HERC6, OLAH, FAM20A, MMP8 | | | | | | | | | |
| OAS1, IFIT1, SIGLEC1, ISG15, | 0.878 | 0.869 | 0.887 | 0.921 | 0.863 | 0.979 | 0.947 | 0.882 | 1 |
| HERC6, FAM20A, RETN, MMP8 | | | | | | | | | |
| RSAD2, SIGLEC1, HERC6, | 0.878 | 0.868 | 0.888 | 0.926 | 0.878 | 0.973 | 0.960 | 0.900 | 1 |
| SLC1A2, IL1R2, OLAH, FAM20A, MMP8 | | | | | | | | | |
| IFIT1, SIGLEC1, ISG15, SLC1A2, | 0.878 | 0.869 | 0.887 | 0.927 | 0.880 | 0.974 | 0.963 | 0.905 | 1 |
| IL1R2, OLAH, FAM20A, MMP8 | | | | | | | | | |
| IFI27, OAS1, IFIT1, ISG15, IL1R2, | 0.878 | 0.869 | 0.887 | 0.920 | 0.864 | 0.977 | 0.951 | 0.898 | 1 |
| OLAH, RETN, MMP8 | | | | | | | | | |
| RSAD2, IFI27, OAS1, ISG15, | 0.878 | 0.869 | 0.887 | 0.928 | 0.877 | 0.979 | 0.932 | 0.860 | 1 |
| HERC6, SLC1A2, OLAH, RETN | | | | | | | | | |
| RSAD2, IFI27, IFIT1, ISG15, | 0.878 | 0.869 | 0.887 | 0.924 | 0.874 | 0.974 | 0.939 | 0.884 | 0.994 |
| HERC6, IL1R2, OLAH, MMP8 | | | | | | | | | |
| RSAD2, IFIT1, SIGLEC1, HERC6, | 0.878 | 0.868 | 0.888 | 0.927 | 0.878 | 0.976 | 0.946 | 0.876 | 1 |
| SLC1A2, OLAH, FAM20A, MMP8 | | | | | | | | | |
| IFI27, OAS1, IFIT1, ISG15, HERC6, | 0.878 | 0.868 | 0.888 | 0.929 | 0.879 | 0.978 | 0.930 | 0.864 | 0.997 |
| SLC1A2, OLAH, MMP8 | | | | | | | | | |
| RSAD2, IFI27, IFIT1, IFI44L, | 0.878 | 0.869 | 0.886 | 0.920 | 0.865 | 0.975 | 0.952 | 0.900 | 1 |
| SIGLEC1, IL1R2, OLAH, RETN | | | | | | | | | |
| OAS1, IFIT1, SIGLEC1, HERC6, | 0.878 | 0.869 | 0.887 | 0.919 | 0.861 | 0.977 | 0.952 | 0.895 | 1 |
| IL1R2, OLAH, FAM20A, RETN | | | | | | | | | |
| RSAD2, IFI27, OAS1, IFIT1, | 0.878 | 0.869 | 0.886 | 0.916 | 0.856 | 0.975 | 0.955 | 0.899 | 1 |
| IFI44L, SIGLEC1, OLAH, RETN | | | | | | | | | |
| IFI27, OAS1, IFI44L, SIGLEC1, | 0.878 | 0.869 | 0.887 | 0.929 | 0.879 | 0.979 | 0.945 | 0.887 | 1 |
| ISG15, SLC1A2, OLAH, MMP8 | | | | | | | | | |
| RSAD2, IFI27, IFIT1, SIGLEC1, | 0.878 | 0.869 | 0.887 | 0.930 | 0.881 | 0.980 | 0.939 | 0.880 | 0.999 |
| ISG15, SLC1A2, OLAH, MMP8 | | | | | | | | | |
| IFI27, IFIT1, SIGLEC1, ISG15, | 0.878 | 0.868 | 0.887 | 0.928 | 0.877 | 0.978 | 0.937 | 0.859 | 1 |
| HERC6, SLC1A2, FAM20A, MMP8 | | | | | | | | | |
| IFI27, IFIT1, IFI44L, ISG15, | 0.878 | 0.869 | 0.887 | 0.928 | 0.880 | 0.976 | 0.927 | 0.840 | 1 |
| SLC1A2, IL1R2, FAM20A, MMP8 | | | | | | | | | |
| IFI27, OAS1, SIGLEC1, ISG15, | 0.878 | 0.868 | 0.887 | 0.929 | 0.878 | 0.981 | 0.954 | 0.897 | 1 |
| SLC1A2, OLAH, RETN, MMP8 | | | | | | | | | |
| RSAD2, IFI27, OAS1, SIGLEC1, | 0.878 | 0.869 | 0.887 | 0.929 | 0.879 | 0.979 | 0.942 | 0.884 | 1 |
| ISG15, SLC1A2, IL1R2, OLAH | | | | | | | | | |
| IFI27, IFIT1, SIGLEC1, HERC6, | 0.878 | 0.868 | 0.887 | 0.927 | 0.878 | 0.977 | 0.945 | 0.890 | 0.999 |
| SLC1A2, IL1R2, OLAH, MMP8 | | | | | | | | | |
| RSAD2, IFIT1, SIGLEC1, HERC6, | 0.878 | 0.868 | 0.887 | 0.921 | 0.864 | 0.978 | 0.952 | 0.896 | 1 |
| OLAH, FAM20A, RETN, MMP8 | | | | | | | | | |
| RSAD2, IFI27, OAS1, IFI44L, | 0.878 | 0.868 | 0.887 | 0.916 | 0.857 | 0.976 | 0.943 | 0.873 | 1 |
| SIGLEC1, HERC6, FAM20A, MMP8 | | | | | | | | | |
| RSAD2, IFI27, OAS1, IFIT1, IL1R2, | 0.878 | 0.868 | 0.887 | 0.922 | 0.866 | 0.977 | 0.952 | 0.900 | 1 |
| OLAH, RETN, MMP8 | | | | | | | | | |
| RSAD2, IFI27, OAS1, IFI44L, | 0.878 | 0.868 | 0.887 | 0.916 | 0.857 | 0.976 | 0.942 | 0.870 | 1 |
| SIGLEC1, ISG15, HERC6, FAM20A | | | | | | | | | |
| IFI27, IFIT1, IFI44L, SIGLEC1, | 0.878 | 0.869 | 0.886 | 0.928 | 0.878 | 0.978 | 0.940 | 0.883 | 0.998 |
| ISG15, SLC1A2, IL1R2, OLAH | | | | | | | | | |
| IFIT1, IFI44L, SIGLEC1, HERC6, | 0.878 | 0.869 | 0.887 | 0.917 | 0.859 | 0.975 | 0.951 | 0.894 | 1 |
| IL1R2, OLAH, FAM20A, RETN | | | | | | | | | |
| IFIT1, SIGLEC1, ISG15, HERC6, | 0.878 | 0.868 | 0.887 | 0.928 | 0.882 | 0.974 | 0.958 | 0.890 | 1 |
| SLC1A2, IL1R2, FAM20A, MMP8 | | | | | | | | | |
| RSAD2, IFI27, OAS1, SIGLEC1, | 0.878 | 0.869 | 0.887 | 0.923 | 0.872 | 0.974 | 0.945 | 0.894 | 0.995 |
| ISG15, IL1R2, OLAH, MMP8 | | | | | | | | | |
| RSAD2, IFIT1, IFI44L, SIGLEC1, | 0.878 | 0.868 | 0.887 | 0.917 | 0.859 | 0.975 | 0.949 | 0.891 | 1 |
| HERC6, OLAH, FAM20A, MMP8 | | | | | | | | | |
| RSAD2, IFI27, OAS1, IFI44L, | 0.878 | 0.869 | 0.886 | 0.930 | 0.880 | 0.981 | 0.948 | 0.892 | 1 |
| SIGLEC1, ISG15, SLC1A2, OLAH | | | | | | | | | |
| RSAD2, IFI27, IFIT1, IFI44L, | 0.878 | 0.869 | 0.886 | 0.926 | 0.875 | 0.976 | 0.942 | 0.889 | 0.996 |
| SIGLEC1, ISG15, IL1R2, OLAH | | | | | | | | | |
| RSAD2, OAS1, IFIT1, SIGLEC1, | 0.878 | 0.868 | 0.887 | 0.922 | 0.865 | 0.978 | 0.968 | 0.918 | 1 |
| IL1R2, FAM20A, RETN, MMP8 | | | | | | | | | |
| IFI27, OAS1, IFIT1, IFI44L, | 0.878 | 0.869 | 0.886 | 0.924 | 0.878 | 0.970 | 0.939 | 0.886 | 0.992 |
| SIGLEC1, ISG15, HERC6, IL1R2 | | | | | | | | | |
| IFI27, OAS1, IFIT1, SIGLEC1, | 0.878 | 0.868 | 0.887 | 0.929 | 0.880 | 0.977 | 0.936 | 0.859 | 1 |
| ISG15, HERC6, SLC1A2, FAM20A | | | | | | | | | |
| IFI27, IFIT1, IFI44L, SIGLEC1, | 0.878 | 0.869 | 0.887 | 0.930 | 0.881 | 0.980 | 0.940 | 0.883 | 0.998 |
| ISG15, HERC6, SLC1A2, OLAH | | | | | | | | | |
| IFIT1, SIGLEC1, HERC6, SLC1A2, | 0.878 | 0.868 | 0.887 | 0.928 | 0.880 | 0.975 | 0.958 | 0.890 | 1 |
| IL1R2, FAM20A, RETN, MMP8 | | | | | | | | | |
| IFI27, OAS1, IFI44L, SIGLEC1, | 0.878 | 0.869 | 0.886 | 0.928 | 0.881 | 0.975 | 0.939 | 0.881 | 0.997 |
| HERC6, SLC1A2, IL1R2, RETN | | | | | | | | | |
| RSAD2, IFIT1, IFI44L, SIGLEC1, | 0.878 | 0.869 | 0.886 | 0.918 | 0.860 | 0.976 | 0.953 | 0.897 | 1 |
| ISG15, HERC6, IL1R2, FAM20A | | | | | | | | | |
| RSAD2, IFI27, IFIT1, IFI44L, | 0.878 | 0.868 | 0.887 | 0.928 | 0.879 | 0.977 | 0.927 | 0.842 | 1 |
| SLC1A2, IL1R2, FAM20A, MMP8 | | | | | | | | | |
| RSAD2, IFI27, OAS1, IFIT1, ISG15, | 0.878 | 0.868 | 0.887 | 0.924 | 0.871 | 0.978 | 0.929 | 0.850 | 1 |
| IL1R2, FAM20A, RETN | | | | | | | | | |
| IFI27, OAS1, SIGLEC1, ISG15, | 0.878 | 0.869 | 0.886 | 0.928 | 0.877 | 0.979 | 0.942 | 0.881 | 1 |
| SLC1A2, IL1R2, OLAH, RETN | | | | | | | | | |
| RSAD2, IFI27, OAS1, IFIT1, | 0.878 | 0.868 | 0.887 | 0.928 | 0.879 | 0.977 | 0.936 | 0.859 | 1 |
| SIGLEC1, HERC6, SLC1A2, FAM20A | | | | | | | | | |
| RSAD2, IFIT1, SIGLEC1, SLC1A2, | 0.878 | 0.868 | 0.887 | 0.927 | 0.879 | 0.975 | 0.962 | 0.906 | 1 |
| IL1R2, OLAH, FAM20A, MMP8 | | | | | | | | | |
| IFI27, OAS1, IFIT1, IFI44L, | 0.878 | 0.868 | 0.887 | 0.926 | 0.875 | 0.977 | 0.934 | 0.867 | 1 |
| HERC6, SLC1A2, OLAH, RETN | | | | | | | | | |
| RSAD2, IFI27, OAS1, SIGLEC1, | 0.877 | 0.868 | 0.887 | 0.920 | 0.861 | 0.979 | 0.959 | 0.905 | 1 |
| ISG15, OLAH, RETN, MMP8 | | | | | | | | | |
| RSAD2, IFIT1, IFI44L, SIGLEC1, | 0.877 | 0.868 | 0.887 | 0.920 | 0.862 | 0.977 | 0.966 | 0.913 | 1 |
| ISG15, IL1R2, FAM20A, MMP8 | | | | | | | | | |
| IFI27, OAS1, IFIT1, SIGLEC1, | 0.877 | 0.868 | 0.886 | 0.929 | 0.878 | 0.979 | 0.943 | 0.883 | 1 |
| ISG15, SLC1A2, OLAH, RETN | | | | | | | | | |
| IFI27, OAS1, SIGLEC1, ISG15, | 0.877 | 0.867 | 0.888 | 0.923 | 0.866 | 0.980 | 0.940 | 0.863 | 1 |
| SLC1A2, FAM20A, RETN, MMP8 | | | | | | | | | |
| RSAD2, IFI27, IFIT1, SIGLEC1, | 0.877 | 0.868 | 0.887 | 0.930 | 0.880 | 0.980 | 0.939 | 0.881 | 0.997 |
| ISG15, HERC6, SLC1A2, OLAH | | | | | | | | | |
| RSAD2, IFI27, IFI44L, SIGLEC1, | 0.877 | 0.868 | 0.887 | 0.926 | 0.875 | 0.977 | 0.943 | 0.888 | 0.999 |
| HERC6, SLC1A2, OLAH, MMP8 | | | | | | | | | |
| OAS1, IFIT1, IFI44L, SIGLEC1, | 0.877 | 0.868 | 0.887 | 0.926 | 0.879 | 0.973 | 0.963 | 0.904 | 1 |
| SLC1A2, IL1R2, FAM20A, MMP8 | | | | | | | | | |
| IFI27, IFIT1, IFI44L, SIGLEC1, | 0.877 | 0.868 | 0.887 | 0.912 | 0.851 | 0.973 | 0.939 | 0.884 | 0.995 |
| ISG15, HERC6, RETN, MMP8 | | | | | | | | | |
| RSAD2, IFI27, IFI44L, HERC6, | 0.877 | 0.868 | 0.887 | 0.928 | 0.877 | 0.979 | 0.941 | 0.881 | 1 |
| SLC1A2, OLAH, RETN, MMP8 | | | | | | | | | |
| RSAD2, IFI27, OAS1, IFI44L, | 0.877 | 0.868 | 0.887 | 0.925 | 0.872 | 0.978 | 0.928 | 0.848 | 1 |
| ISG15, IL1R2, FAM20A, RETN | | | | | | | | | |
| RSAD2, IFI27, OAS1, IFIT1, ISG15, | 0.877 | 0.868 | 0.887 | 0.919 | 0.859 | 0.978 | 0.950 | 0.893 | 1 |
| OLAH, RETN, MMP8 | | | | | | | | | |
| RSAD2, IFI27, IFIT1, SLC1A2, | 0.877 | 0.868 | 0.887 | 0.932 | 0.883 | 0.981 | 0.930 | 0.850 | 1 |
| IL1R2, FAM20A, RETN, MMP8 | | | | | | | | | |
| RSAD2, IFI27, OAS1, HERC6, | 0.877 | 0.868 | 0.887 | 0.929 | 0.880 | 0.977 | 0.935 | 0.869 | 1 |
| SLC1A2, IL1R2, OLAH, MMP8 | | | | | | | | | |
| RSAD2, IFI27, SIGLEC1, ISG15, | 0.877 | 0.868 | 0.887 | 0.929 | 0.879 | 0.979 | 0.941 | 0.883 | 0.999 |
| HERC6, SLC1A2, OLAH, RETN | | | | | | | | | |
| RSAD2, IFI27, IFI44L, SIGLEC1, | 0.877 | 0.868 | 0.886 | 0.921 | 0.864 | 0.979 | 0.957 | 0.905 | 1 |
| ISG15, OLAH, RETN, MMP8 | | | | | | | | | |
| IFI27, IFI44L, ISG15, HERC6, | 0.877 | 0.868 | 0.887 | 0.927 | 0.876 | 0.979 | 0.937 | 0.874 | 1 |
| SLC1A2, OLAH, RETN, MMP8 | | | | | | | | | |
| RSAD2, IFI27, IFIT1, IFI44L, | 0.877 | 0.868 | 0.887 | 0.926 | 0.870 | 0.983 | 0.922 | 0.841 | 1 |
| SLC1A2, OLAH, FAM20A, RETN | | | | | | | | | |
| RSAD2, IFI27, OAS1, HERC6, | 0.877 | 0.868 | 0.887 | 0.931 | 0.886 | 0.976 | 0.921 | 0.829 | 1 |
| SLC1A2, IL1R2, FAM20A, MMP8 | | | | | | | | | |
| IFI27, OAS1, IFIT1, SLC1A2, | 0.877 | 0.868 | 0.887 | 0.932 | 0.883 | 0.981 | 0.928 | 0.847 | 1 |
| IL1R2, FAM20A, RETN, MMP8 | | | | | | | | | |
| RSAD2, IFI27, OAS1, IFIT1, | 0.877 | 0.868 | 0.886 | 0.919 | 0.863 | 0.974 | 0.950 | 0.893 | 1 |
| SIGLEC1, IL1R2, OLAH, RETN | | | | | | | | | |
| IFI27, IFIT1, IFI44L, ISG15, IL1R2, | 0.877 | 0.868 | 0.887 | 0.918 | 0.861 | 0.975 | 0.948 | 0.894 | 1 |
| OLAH, RETN, MMP8 | | | | | | | | | |
| RSAD2, IFI27, OAS1, IFIT1, | 0.877 | 0.868 | 0.887 | 0.924 | 0.867 | 0.981 | 0.924 | 0.844 | 1 |
| SLC1A2, OLAH, FAM20A, RETN | | | | | | | | | |
| IFI27, OAS1, IFIT1, IFI44L, | 0.877 | 0.868 | 0.887 | 0.928 | 0.880 | 0.976 | 0.926 | 0.840 | 1 |
| SLC1A2, IL1R2, FAM20A, MMP8 | | | | | | | | | |
| SIGLEC1, ISG15, HERC6, IL1R2, | 0.877 | 0.868 | 0.887 | 0.916 | 0.857 | 0.976 | 0.965 | 0.911 | 1 |
| OLAH, FAM20A, RETN, MMP8 | | | | | | | | | |
| RSAD2, SIGLEC1, ISG15, HERC6, | 0.877 | 0.868 | 0.887 | 0.920 | 0.863 | 0.977 | 0.958 | 0.896 | 1 |
| IL1R2, FAM20A, RETN, MMP8 | | | | | | | | | |
| IFI27, OAS1, IFI44L, SIGLEC1, | 0.877 | 0.869 | 0.885 | 0.922 | 0.876 | 0.969 | 0.940 | 0.888 | 0.993 |
| ISG15, HERC6, IL1R2, MMP8 | | | | | | | | | |
| IFIT1, IFI44L, SIGLEC1, ISG15, | 0.877 | 0.868 | 0.886 | 0.926 | 0.880 | 0.973 | 0.961 | 0.897 | 1 |
| SLC1A2, IL1R2, FAM20A, MMP8 | | | | | | | | | |
| IFI27, OAS1, IFI44L, ISG15, IL1R2, | 0.877 | 0.868 | 0.886 | 0.924 | 0.868 | 0.979 | 0.946 | 0.890 | 1 |
| OLAH, RETN, MMP8 | | | | | | | | | |
| RSAD2, IFI27, IFIT1, SIGLEC1, | 0.877 | 0.868 | 0.887 | 0.920 | 0.860 | 0.979 | 0.958 | 0.905 | 1 |
| ISG15, OLAH, RETN, MMP8 | | | | | | | | | |
| IFI27, IFIT1, SIGLEC1, SLC1A2, | 0.877 | 0.868 | 0.887 | 0.929 | 0.878 | 0.979 | 0.959 | 0.909 | 1 |
| IL1R2, OLAH, RETN, MMP8 | | | | | | | | | |
| IFI27, IFI44L, ISG15, HERC6, | 0.877 | 0.867 | 0.887 | 0.930 | 0.883 | 0.976 | 0.927 | 0.840 | 1 |
| SLC1A2, IL1R2, FAM20A, MMP8 | | | | | | | | | |
| OAS1, SIGLEC1, HERC6, SLC1A2, | 0.877 | 0.867 | 0.887 | 0.923 | 0.874 | 0.972 | 0.960 | 0.900 | 1 |
| IL1R2, OLAH, FAM20A, MMP8 | | | | | | | | | |
| RSAD2, IFI27, IFIT1, SIGLEC1, | 0.877 | 0.868 | 0.886 | 0.927 | 0.878 | 0.977 | 0.947 | 0.893 | 1 |
| SLC1A2, IL1R2, OLAH, MMP8 | | | | | | | | | |
| RSAD2, IFI27, IFIT1, IFI44L, | 0.877 | 0.868 | 0.887 | 0.928 | 0.879 | 0.976 | 0.925 | 0.838 | 1 |
| HERC6, SLC1A2, IL1R2, FAM20A | | | | | | | | | |
| IFI27, IFIT1, IFI44L, SIGLEC1, | 0.877 | 0.869 | 0.886 | 0.927 | 0.877 | 0.976 | 0.950 | 0.899 | 1 |
| SLC1A2, IL1R2, OLAH, MMP8 | | | | | | | | | |
| RSAD2, IFI27, IFIT1, SIGLEC1, | 0.877 | 0.868 | 0.887 | 0.929 | 0.879 | 0.978 | 0.945 | 0.889 | 1 |
| HERC6, SLC1A2, OLAH, RETN | | | | | | | | | |
| OAS1, IFI44L, SIGLEC1, HERC6, | 0.877 | 0.868 | 0.887 | 0.919 | 0.862 | 0.976 | 0.962 | 0.905 | 1 |
| IL1R2, FAM20A, RETN, MMP8 | | | | | | | | | |
| RSAD2, IFI27, OAS1, IFI44L, | 0.877 | 0.867 | 0.887 | 0.930 | 0.884 | 0.975 | 0.926 | 0.839 | 1 |
| HERC6, SLC1A2, IL1R2, FAM20A | | | | | | | | | |
| RSAD2, IFI27, OAS1, IFIT1, | 0.877 | 0.867 | 0.887 | 0.914 | 0.849 | 0.980 | 0.946 | 0.877 | 1 |
| IFI44L, SIGLEC1, FAM20A, MMP8 | | | | | | | | | |
| RSAD2, IFI27, IFI44L, SIGLEC1, | 0.877 | 0.868 | 0.886 | 0.931 | 0.881 | 0.980 | 0.940 | 0.883 | 0.998 |
| ISG15, HERC6, SLC1A2, OLAH | | | | | | | | | |
| RSAD2, IFI27, OAS1, IFI44L, | 0.877 | 0.868 | 0.886 | 0.929 | 0.879 | 0.979 | 0.947 | 0.892 | 1 |
| SIGLEC1, HERC6, SLC1A2, OLAH | | | | | | | | | |
| RSAD2, IFI27, OAS1, IFIT1, | 0.877 | 0.868 | 0.886 | 0.925 | 0.875 | 0.976 | 0.951 | 0.897 | 1 |
| SIGLEC1, SLC1A2, IL1R2, OLAH | | | | | | | | | |
| IFI27, OAS1, IFIT1, HERC6, | 0.877 | 0.867 | 0.887 | 0.932 | 0.884 | 0.980 | 0.926 | 0.842 | 1 |
| SLC1A2, IL1R2, FAM20A, RETN | | | | | | | | | |
| IFI27, OAS1, ISG15, HERC6, | 0.877 | 0.867 | 0.887 | 0.930 | 0.884 | 0.977 | 0.925 | 0.839 | 1 |
| SLC1A2, IL1R2, FAM20A, MMP8 | | | | | | | | | |
| RSAD2, IFI27, IFIT1, IFI44L, | 0.877 | 0.869 | 0.886 | 0.925 | 0.874 | 0.976 | 0.935 | 0.876 | 0.993 |
| ISG15, HERC6, IL1R2, OLAH | | | | | | | | | |
| RSAD2, IFI27, OAS1, IFI44L, | 0.877 | 0.868 | 0.886 | 0.918 | 0.860 | 0.977 | 0.946 | 0.885 | 1 |
| SIGLEC1, ISG15, OLAH, RETN | | | | | | | | | |
| IFI27, ISG15, HERC6, SLC1A2, | 0.877 | 0.867 | 0.887 | 0.934 | 0.886 | 0.982 | 0.929 | 0.844 | 1 |
| IL1R2, FAM20A, RETN, MMP8 | | | | | | | | | |
| IFIT1, SIGLEC1, ISG15, HERC6, | 0.877 | 0.868 | 0.886 | 0.919 | 0.862 | 0.977 | 0.952 | 0.895 | 1 |
| IL1R2, OLAH, FAM20A, RETN | | | | | | | | | |
| OAS1, SIGLEC1, ISG15, HERC6, | 0.877 | 0.868 | 0.886 | 0.917 | 0.859 | 0.975 | 0.962 | 0.904 | 1 |
| IL1R2, FAM20A, RETN, MMP8 | | | | | | | | | |
| RSAD2, IFI27, IFIT1, SIGLEC1, | 0.877 | 0.868 | 0.886 | 0.927 | 0.877 | 0.978 | 0.935 | 0.856 | 1 |
| HERC6, SLC1A2, FAM20A, MMP8 | | | | | | | | | |
| RSAD2, IFI27, SIGLEC1, SLC1A2, | 0.877 | 0.868 | 0.887 | 0.929 | 0.879 | 0.979 | 0.958 | 0.906 | 1 |
| IL1R2, OLAH, RETN, MMP8 | | | | | | | | | |
| RSAD2, OAS1, SIGLEC1, HERC6, | 0.877 | 0.867 | 0.887 | 0.924 | 0.876 | 0.972 | 0.959 | 0.895 | 1 |
| SLC1A2, IL1R2, FAM20A, MMP8 | | | | | | | | | |
| IFI27, OAS1, IFIT1, ISG15, HERC6, | 0.877 | 0.867 | 0.887 | 0.930 | 0.883 | 0.977 | 0.924 | 0.838 | 1 |
| SLC1A2, IL1R2, FAM20A | | | | | | | | | |
| RSAD2, IFI27, OAS1, IFIT1, | 0.877 | 0.869 | 0.886 | 0.921 | 0.869 | 0.973 | 0.946 | 0.892 | 0.999 |
| SIGLEC1, ISG15, IL1R2, OLAH | | | | | | | | | |
| IFI27, OAS1, IFI44L, SIGLEC1, | 0.877 | 0.868 | 0.887 | 0.928 | 0.876 | 0.980 | 0.957 | 0.901 | 1 |
| SLC1A2, OLAH, RETN, MMP8 | | | | | | | | | |
| RSAD2, IFI27, OAS1, IFIT1, | 0.877 | 0.869 | 0.886 | 0.921 | 0.874 | 0.969 | 0.951 | 0.905 | 0.997 |
| SIGLEC1, HERC6, IL1R2, MMP8 | | | | | | | | | |
| IFI27, IFI44L, SIGLEC1, HERC6, | 0.877 | 0.868 | 0.887 | 0.924 | 0.873 | 0.976 | 0.948 | 0.893 | 1 |
| SLC1A2, IL1R2, OLAH, RETN | | | | | | | | | |
| RSAD2, OAS1, IFIT1, IFI44L, | 0.877 | 0.868 | 0.886 | 0.915 | 0.856 | 0.975 | 0.943 | 0.884 | 1 |
| SIGLEC1, HERC6, OLAH, FAM20A | | | | | | | | | |
| RSAD2, IFI27, OAS1, HERC6, | 0.877 | 0.868 | 0.886 | 0.932 | 0.885 | 0.980 | 0.923 | 0.834 | 1 |
| SLC1A2, IL1R2, FAM20A, RETN | | | | | | | | | |
| IFI27, OAS1, IFI44L, HERC6, | 0.877 | 0.867 | 0.887 | 0.932 | 0.884 | 0.980 | 0.925 | 0.840 | 1 |
| SLC1A2, IL1R2, FAM20A, RETN | | | | | | | | | |
| IFI27, IFI44L, ISG15, SLC1A2, | 0.877 | 0.868 | 0.886 | 0.927 | 0.876 | 0.979 | 0.936 | 0.870 | 1 |
| IL1R2, OLAH, RETN, MMP8 | | | | | | | | | |
| RSAD2, OAS1, SIGLEC1, SLC1A2, | 0.877 | 0.867 | 0.887 | 0.924 | 0.875 | 0.973 | 0.959 | 0.898 | 1 |
| IL1R2, OLAH, FAM20A, MMP8 | | | | | | | | | |
| RSAD2, IFI27, OAS1, IFIT1, | 0.877 | 0.868 | 0.886 | 0.929 | 0.878 | 0.980 | 0.943 | 0.886 | 1 |
| SIGLEC1, ISG15, SLC1A2, OLAH | | | | | | | | | |
| IFI27, IFIT1, IFI44L, SIGLEC1, | 0.877 | 0.868 | 0.887 | 0.928 | 0.876 | 0.980 | 0.959 | 0.908 | 1 |
| SLC1A2, OLAH, RETN, MMP8 | | | | | | | | | |
| IFI27, IFI44L, SIGLEC1, ISG15, | 0.877 | 0.868 | 0.886 | 0.929 | 0.880 | 0.977 | 0.942 | 0.887 | 0.997 |
| HERC6, SLC1A2, IL1R2, OLAH | | | | | | | | | |
| RSAD2, IFI27, OAS1, SIGLEC1, | 0.877 | 0.868 | 0.886 | 0.928 | 0.877 | 0.979 | 0.950 | 0.888 | 1 |
| SLC1A2, IL1R2, OLAH, RETN | | | | | | | | | |
| RSAD2, IFI27, OAS1, ISG15, | 0.877 | 0.868 | 0.886 | 0.929 | 0.878 | 0.979 | 0.930 | 0.863 | 0.998 |
| HERC6, SLC1A2, OLAH, MMP8 | | | | | | | | | |
| RSAD2, IFI27, OAS1, IFI44L, | 0.877 | 0.868 | 0.886 | 0.927 | 0.875 | 0.979 | 0.933 | 0.861 | 1 |
| HERC6, SLC1A2, OLAH, RETN | | | | | | | | | |
| IFI44L, SIGLEC1, HERC6, | 0.877 | 0.867 | 0.887 | 0.923 | 0.874 | 0.973 | 0.959 | 0.897 | 1 |
| SLC1A2, IL1R2, OLAH, FAM20A, MMP8 | | | | | | | | | |
| IFI27, OAS1, IFIT1, IFI44L, | 0.877 | 0.868 | 0.886 | 0.929 | 0.879 | 0.979 | 0.945 | 0.887 | 1 |
| SIGLEC1, ISG15, SLC1A2, OLAH | | | | | | | | | |
| IFIT1, IFI44L, SIGLEC1, SLC1A2, | 0.877 | 0.867 | 0.887 | 0.924 | 0.872 | 0.977 | 0.949 | 0.890 | 1 |
| OLAH, FAM20A, RETN, MMP8 | | | | | | | | | |
| RSAD2, IFI27, OAS1, IFIT1, | 0.877 | 0.869 | 0.885 | 0.919 | 0.862 | 0.975 | 0.951 | 0.901 | 1 |
| IFI44L, SIGLEC1, OLAH, MMP8 | | | | | | | | | |
| OAS1, IFIT1, IFI44L, SIGLEC1, | 0.877 | 0.868 | 0.886 | 0.915 | 0.855 | 0.976 | 0.941 | 0.881 | 1 |
| HERC6, OLAH, FAM20A, RETN | | | | | | | | | |
| OAS1, IFIT1, IFI44L, SIGLEC1, | 0.877 | 0.868 | 0.886 | 0.918 | 0.861 | 0.976 | 0.950 | 0.893 | 1 |
| HERC6, IL1R2, FAM20A, RETN | | | | | | | | | |
| RSAD2, IFIT1, SIGLEC1, ISG15, | 0.877 | 0.867 | 0.887 | 0.916 | 0.858 | 0.975 | 0.953 | 0.899 | 1 |
| HERC6, OLAH, FAM20A, MMP8 | | | | | | | | | |
| RSAD2, IFI27, IFIT1, IFI44L, | 0.877 | 0.868 | 0.886 | 0.924 | 0.871 | 0.976 | 0.934 | 0.874 | 0.993 |
| ISG15, HERC6, OLAH, MMP8 | | | | | | | | | |
| IFI27, IFIT1, ISG15, HERC6, | 0.877 | 0.867 | 0.887 | 0.927 | 0.876 | 0.979 | 0.941 | 0.881 | 1 |
| SLC1A2, OLAH, RETN, MMP8 | | | | | | | | | |
| RSAD2, IFI27, OAS1, SIGLEC1, | 0.877 | 0.868 | 0.886 | 0.926 | 0.877 | 0.976 | 0.951 | 0.897 | 1 |
| SLC1A2, IL1R2, OLAH, MMP8 | | | | | | | | | |
| IFI27, OAS1, IFIT1, ISG15, HERC6, | 0.877 | 0.867 | 0.887 | 0.927 | 0.878 | 0.977 | 0.929 | 0.862 | 0.997 |
| SLC1A2, IL1R2, OLAH | | | | | | | | | |
| IFI27, OAS1, IFI44L, SIGLEC1, | 0.877 | 0.868 | 0.886 | 0.923 | 0.870 | 0.977 | 0.939 | 0.875 | 1 |
| ISG15, HERC6, SLC1A2, RETN | | | | | | | | | |
| RSAD2, IFI27, IFIT1, SIGLEC1, | 0.877 | 0.868 | 0.886 | 0.926 | 0.871 | 0.981 | 0.938 | 0.862 | 1 |
| ISG15, SLC1A2, FAM20A, RETN | | | | | | | | | |
| IFI27, ISG15, HERC6, SLC1A2, | 0.877 | 0.868 | 0.886 | 0.929 | 0.879 | 0.979 | 0.941 | 0.881 | 1 |
| IL1R2, OLAH, RETN, MMP8 | | | | | | | | | |
| RSAD2, IFI27, IFI44L, SIGLEC1, | 0.877 | 0.867 | 0.887 | 0.927 | 0.875 | 0.980 | 0.943 | 0.870 | 1 |
| HERC6, SLC1A2, FAM20A, RETN | | | | | | | | | |
| RSAD2, IFI44L, SIGLEC1, ISG15, | 0.877 | 0.868 | 0.886 | 0.918 | 0.861 | 0.975 | 0.960 | 0.900 | 1 |
| HERC6, IL1R2, FAM20A, MMP8 | | | | | | | | | |
| RSAD2, IFI27, IFIT1, IFI44L, | 0.877 | 0.867 | 0.887 | 0.924 | 0.869 | 0.980 | 0.940 | 0.868 | 1 |
| SIGLEC1, SLC1A2, FAM20A, RETN | | | | | | | | | |
| IFI44L, SIGLEC1, ISG15, IL1R2, | 0.877 | 0.868 | 0.886 | 0.917 | 0.858 | 0.975 | 0.964 | 0.911 | 1 |
| OLAH, FAM20A, RETN, MMP8 | | | | | | | | | |
| RSAD2, IFI27, OAS1, SIGLEC1, | 0.877 | 0.867 | 0.886 | 0.927 | 0.874 | 0.980 | 0.957 | 0.900 | 1 |
| SLC1A2, OLAH, RETN, MMP8 | | | | | | | | | |
| IFI27, OAS1, IFIT1, SIGLEC1, | 0.877 | 0.869 | 0.885 | 0.921 | 0.874 | 0.968 | 0.949 | 0.902 | 0.996 |
| ISG15, HERC6, IL1R2, MMP8 | | | | | | | | | |
| RSAD2, IFI27, OAS1, IFIT1, | 0.877 | 0.868 | 0.886 | 0.922 | 0.870 | 0.974 | 0.942 | 0.889 | 0.995 |
| IFI44L, IL1R2, OLAH, MMP8 | | | | | | | | | |
| IFIT1, SIGLEC1, HERC6, SLC1A2, | 0.877 | 0.867 | 0.887 | 0.927 | 0.877 | 0.978 | 0.952 | 0.884 | 1 |
| OLAH, FAM20A, RETN, MMP8 | | | | | | | | | |
| IFI44L, SIGLEC1, HERC6, IL1R2, | 0.877 | 0.867 | 0.887 | 0.920 | 0.863 | 0.977 | 0.960 | 0.900 | 1 |
| OLAH, FAM20A, RETN, MMP8 | | | | | | | | | |
| IFI27, OAS1, IFIT1, IFI44L, | 0.877 | 0.868 | 0.886 | 0.926 | 0.874 | 0.978 | 0.948 | 0.886 | 1 |
| SIGLEC1, SLC1A2, OLAH, RETN | | | | | | | | | |
| RSAD2, IFI27, IFI44L, SIGLEC1, | 0.877 | 0.868 | 0.885 | 0.930 | 0.880 | 0.980 | 0.942 | 0.885 | 1 |
| ISG15, SLC1A2, OLAH, MMP8 | | | | | | | | | |
| IFI27, IFI44L, HERC6, SLC1A2, | 0.877 | 0.867 | 0.886 | 0.926 | 0.874 | 0.978 | 0.937 | 0.873 | 1 |
| IL1R2, OLAH, RETN, MMP8 | | | | | | | | | |
| IFI27, IFI44L, SIGLEC1, HERC6, | 0.877 | 0.868 | 0.886 | 0.927 | 0.878 | 0.976 | 0.951 | 0.902 | 1 |
| SLC1A2, IL1R2, OLAH, MMP8 | | | | | | | | | |
| RSAD2, IFIT1, SIGLEC1, IL1R2, | 0.877 | 0.868 | 0.886 | 0.921 | 0.864 | 0.978 | 0.968 | 0.918 | 1 |
| OLAH, FAM20A, RETN, MMP8 | | | | | | | | | |
| RSAD2, IFI27, OAS1, IFIT1, | 0.877 | 0.868 | 0.885 | 0.926 | 0.874 | 0.978 | 0.953 | 0.900 | 1 |
| IFI44L, SIGLEC1, SLC1A2, OLAH | | | | | | | | | |
| IFI27, SIGLEC1, HERC6, SLC1A2, | 0.877 | 0.867 | 0.886 | 0.927 | 0.876 | 0.979 | 0.959 | 0.910 | 1 |
| IL1R2, OLAH, RETN, MMP8 | | | | | | | | | |
| RSAD2, IFI27, IFIT1, SIGLEC1, | 0.877 | 0.868 | 0.886 | 0.929 | 0.879 | 0.978 | 0.939 | 0.880 | 0.998 |
| ISG15, SLC1A2, IL1R2, OLAH | | | | | | | | | |
| RSAD2, IFI27, IFIT1, IFI44L, | 0.877 | 0.869 | 0.885 | 0.923 | 0.875 | 0.971 | 0.941 | 0.890 | 0.993 |
| SIGLEC1, HERC6, IL1R2, MMP8 | | | | | | | | | |
| IFI27, OAS1, IFI44L, ISG15, | 0.877 | 0.867 | 0.886 | 0.928 | 0.878 | 0.978 | 0.930 | 0.861 | 1.000 |
| HERC6, SLC1A2, OLAH, RETN | | | | | | | | | |
| RSAD2, IFI27, OAS1, IFI44L, | 0.877 | 0.867 | 0.886 | 0.927 | 0.870 | 0.984 | 0.924 | 0.845 | 1 |
| SLC1A2, OLAH, FAM20A, RETN | | | | | | | | | |
| IFI27, OAS1, SIGLEC1, ISG15, | 0.877 | 0.867 | 0.886 | 0.922 | 0.866 | 0.977 | 0.948 | 0.885 | 1 |
| HERC6, SLC1A2, RETN, MMP8 | | | | | | | | | |
| IFI27, IFIT1, IFI44L, HERC6, | 0.877 | 0.867 | 0.886 | 0.924 | 0.870 | 0.978 | 0.940 | 0.879 | 1 |
| SLC1A2, OLAH, RETN, MMP8 | | | | | | | | | |
| RSAD2, IFI27, SIGLEC1, ISG15, | 0.877 | 0.867 | 0.887 | 0.925 | 0.871 | 0.979 | 0.943 | 0.870 | 1 |
| HERC6, SLC1A2, FAM20A, RETN | | | | | | | | | |
| RSAD2, IFI27, IFI44L, ISG15, | 0.877 | 0.867 | 0.887 | 0.930 | 0.883 | 0.976 | 0.926 | 0.839 | 1 |
| SLC1A2, IL1R2, FAM20A, MMP8 | | | | | | | | | |
| RSAD2, IFI27, OAS1, IFIT1, | 0.877 | 0.867 | 0.886 | 0.927 | 0.877 | 0.978 | 0.935 | 0.869 | 1 |
| HERC6, SLC1A2, OLAH, MMP8 | | | | | | | | | |
| RSAD2, IFI27, SIGLEC1, ISG15, | 0.877 | 0.867 | 0.886 | 0.928 | 0.876 | 0.980 | 0.953 | 0.898 | 1 |
| SLC1A2, OLAH, RETN, MMP8 | | | | | | | | | |
| IFI27, IFIT1, IFI44L, SIGLEC1, | 0.877 | 0.867 | 0.886 | 0.922 | 0.865 | 0.978 | 0.941 | 0.867 | 1 |
| ISG15, SLC1A2, FAM20A, MMP8 | | | | | | | | | |
| IFI27, IFIT1, IFI44L, SIGLEC1, | 0.877 | 0.869 | 0.885 | 0.923 | 0.875 | 0.971 | 0.941 | 0.890 | 0.993 |
| ISG15, HERC6, IL1R2, MMP8 | | | | | | | | | |
| RSAD2, IFI27, ISG15, HERC6, | 0.877 | 0.868 | 0.886 | 0.929 | 0.879 | 0.978 | 0.928 | 0.861 | 0.995 |
| SLC1A2, IL1R2, OLAH, MMP8 | | | | | | | | | |
| RSAD2, IFI27, OAS1, SIGLEC1, | 0.877 | 0.868 | 0.886 | 0.919 | 0.863 | 0.975 | 0.951 | 0.896 | 1 |
| ISG15, IL1R2, OLAH, RETN | | | | | | | | | |
| RSAD2, IFI27, OAS1, IFI44L, | 0.877 | 0.867 | 0.886 | 0.923 | 0.867 | 0.978 | 0.951 | 0.897 | 1 |
| IL1R2, OLAH, RETN, MMP8 | | | | | | | | | |
| IFI27, SIGLEC1, ISG15, HERC6, | 0.877 | 0.867 | 0.886 | 0.929 | 0.879 | 0.979 | 0.939 | 0.877 | 1 |
| SLC1A2, IL1R2, OLAH, RETN | | | | | | | | | |
| IFI27, OAS1, IFI44L, SIGLEC1, | 0.877 | 0.868 | 0.886 | 0.929 | 0.879 | 0.980 | 0.946 | 0.885 | 1 |
| ISG15, SLC1A2, OLAH, RETN | | | | | | | | | |
| RSAD2, IFI27, IFIT1, SIGLEC1, | 0.877 | 0.867 | 0.886 | 0.927 | 0.877 | 0.978 | 0.939 | 0.882 | 0.996 |
| HERC6, SLC1A2, OLAH, MMP8 | | | | | | | | | |
| RSAD2, IFI27, IFIT1, HERC6, | 0.877 | 0.867 | 0.886 | 0.927 | 0.878 | 0.976 | 0.932 | 0.867 | 0.996 |
| SLC1A2, IL1R2, OLAH, MMP8 | | | | | | | | | |
| RSAD2, IFI27, IFI44L, SIGLEC1, | 0.877 | 0.868 | 0.885 | 0.928 | 0.877 | 0.979 | 0.946 | 0.887 | 1 |
| ISG15, SLC1A2, OLAH, RETN | | | | | | | | | |
| RSAD2, IFI27, OAS1, IFI44L, | 0.877 | 0.868 | 0.885 | 0.924 | 0.877 | 0.972 | 0.943 | 0.893 | 0.994 |
| SIGLEC1, ISG15, HERC6, IL1R2 | | | | | | | | | |
| OAS1, IFIT1, IFI44L, SIGLEC1, | 0.877 | 0.867 | 0.886 | 0.919 | 0.862 | 0.977 | 0.941 | 0.877 | 1 |
| ISG15, HERC6, FAM20A, RETN | | | | | | | | | |
| RSAD2, IFI27, IFIT1, SIGLEC1, | 0.877 | 0.867 | 0.886 | 0.929 | 0.878 | 0.981 | 0.957 | 0.903 | 1 |
| SLC1A2, OLAH, RETN, MMP8 | | | | | | | | | |
| RSAD2, IFI27, SIGLEC1, ISG15, | 0.877 | 0.868 | 0.886 | 0.929 | 0.878 | 0.979 | 0.942 | 0.883 | 1 |
| SLC1A2, IL1R2, OLAH, RETN | | | | | | | | | |
| IFI27, OAS1, IFIT1, IFI44L, ISG15, | 0.877 | 0.867 | 0.886 | 0.919 | 0.859 | 0.978 | 0.950 | 0.893 | 1 |
| OLAH, RETN, MMP8 | | | | | | | | | |
| RSAD2, SIGLEC1, HERC6, IL1R2, | 0.877 | 0.867 | 0.886 | 0.920 | 0.864 | 0.977 | 0.961 | 0.905 | 1 |
| OLAH, FAM20A, RETN, MMP8 | | | | | | | | | |
| RSAD2, IFI27, ISG15, HERC6, | 0.877 | 0.867 | 0.886 | 0.931 | 0.885 | 0.977 | 0.926 | 0.839 | 1 |
| SLC1A2, IL1R2, FAM20A, MMP8 | | | | | | | | | |
| RSAD2, IFI27, OAS1, IFIT1, | 0.877 | 0.868 | 0.886 | 0.928 | 0.876 | 0.980 | 0.947 | 0.891 | 1 |
| SIGLEC1, SLC1A2, OLAH, MMP8 | | | | | | | | | |
| RSAD2, IFI27, OAS1, ISG15, | 0.877 | 0.867 | 0.886 | 0.931 | 0.886 | 0.976 | 0.921 | 0.832 | 1 |
| HERC6, SLC1A2, IL1R2, FAM20A | | | | | | | | | |
| RSAD2, IFI27, IFIT1, SIGLEC1, | 0.877 | 0.867 | 0.886 | 0.927 | 0.878 | 0.977 | 0.936 | 0.857 | 1 |
| ISG15, HERC6, SLC1A2, FAM20A | | | | | | | | | |
| RSAD2, IFI27, OAS1, IFIT1, | 0.877 | 0.868 | 0.885 | 0.919 | 0.863 | 0.976 | 0.941 | 0.885 | 0.997 |
| IFI44L, SIGLEC1, ISG15, OLAH | | | | | | | | | |
| RSAD2, IFI27, OAS1, IFI44L, | 0.877 | 0.867 | 0.886 | 0.919 | 0.860 | 0.979 | 0.936 | 0.868 | 1 |
| ISG15, HERC6, RETN, MMP8 | | | | | | | | | |
| IFI27, OAS1, IFIT1, IFI44L, | 0.877 | 0.867 | 0.886 | 0.927 | 0.877 | 0.978 | 0.948 | 0.892 | 1 |
| SIGLEC1, SLC1A2, OLAH, MMP8 | | | | | | | | | |
| IFI27, IFIT1, ISG15, HERC6, | 0.877 | 0.867 | 0.886 | 0.928 | 0.879 | 0.977 | 0.929 | 0.864 | 0.995 |
| SLC1A2, IL1R2, OLAH, MMP8 | | | | | | | | | |
| RSAD2, IFI27, IFI44L, ISG15, | 0.877 | 0.867 | 0.886 | 0.929 | 0.881 | 0.976 | 0.925 | 0.838 | 1 |
| HERC6, SLC1A2, IL1R2, FAM20A | | | | | | | | | |
| RSAD2, IFI27, OAS1, IFIT1, | 0.877 | 0.867 | 0.886 | 0.929 | 0.880 | 0.977 | 0.937 | 0.873 | 1 |
| HERC6, SLC1A2, IL1R2, OLAH | | | | | | | | | |
| IFI27, OAS1, IFI44L, SIGLEC1, | 0.877 | 0.868 | 0.885 | 0.924 | 0.872 | 0.976 | 0.962 | 0.922 | 1 |
| ISG15, IL1R2, RETN, MMP8 | | | | | | | | | |
| OAS1, IFIT1, SIGLEC1, ISG15, | 0.877 | 0.867 | 0.886 | 0.919 | 0.863 | 0.976 | 0.953 | 0.897 | 1 |
| HERC6, IL1R2, FAM20A, RETN | | | | | | | | | |
| OAS1, IFIT1, IFI44L, SIGLEC1, | 0.876 | 0.866 | 0.887 | 0.925 | 0.875 | 0.975 | 0.928 | 0.856 | 1 |
| HERC6, SLC1A2, OLAH, FAM20A | | | | | | | | | |
| RSAD2, IFI27, IFIT1, IFI44L, | 0.876 | 0.867 | 0.886 | 0.922 | 0.867 | 0.977 | 0.950 | 0.895 | 1 |
| IL1R2, OLAH, RETN, MMP8 | | | | | | | | | |
| RSAD2, IFI27, OAS1, ISG15, | 0.876 | 0.867 | 0.886 | 0.929 | 0.880 | 0.977 | 0.928 | 0.859 | 0.997 |
| HERC6, SLC1A2, IL1R2, OLAH | | | | | | | | | |
| RSAD2, IFI27, OAS1, IFI44L, | 0.876 | 0.868 | 0.885 | 0.922 | 0.867 | 0.977 | 0.942 | 0.875 | 1 |
| SIGLEC1, HERC6, SLC1A2, RETN | | | | | | | | | |
| RSAD2, IFI27, IFIT1, SIGLEC1, | 0.876 | 0.867 | 0.886 | 0.928 | 0.879 | 0.977 | 0.941 | 0.886 | 0.997 |
| HERC6, SLC1A2, IL1R2, OLAH | | | | | | | | | |
| RSAD2, IFI27, IFIT1, SIGLEC1, | 0.876 | 0.867 | 0.886 | 0.925 | 0.874 | 0.977 | 0.950 | 0.894 | 1 |
| SLC1A2, IL1R2, OLAH, RETN | | | | | | | | | |
| RSAD2, IFI27, IFI44L, SIGLEC1, | 0.876 | 0.867 | 0.886 | 0.929 | 0.877 | 0.980 | 0.959 | 0.907 | 1 |
| SLC1A2, OLAH, RETN, MMP8 | | | | | | | | | |
| RSAD2, IFI27, ISG15, HERC6, | 0.876 | 0.867 | 0.886 | 0.933 | 0.885 | 0.981 | 0.925 | 0.840 | 1 |
| SLC1A2, IL1R2, FAM20A, RETN | | | | | | | | | |
| RSAD2, IFI27, SIGLEC1, ISG15, | 0.876 | 0.866 | 0.887 | 0.926 | 0.870 | 0.982 | 0.943 | 0.868 | 1 |
| SLC1A2, FAM20A, RETN, MMP8 | | | | | | | | | |
| IFI27, OAS1, IFIT1, SLC1A2, | 0.876 | 0.867 | 0.886 | 0.926 | 0.874 | 0.977 | 0.941 | 0.879 | 1 |
| IL1R2, OLAH, RETN, MMP8 | | | | | | | | | |
| RSAD2, SIGLEC1, ISG15, HERC6, | 0.876 | 0.867 | 0.886 | 0.926 | 0.880 | 0.973 | 0.959 | 0.893 | 1 |
| SLC1A2, IL1R2, FAM20A, MMP8 | | | | | | | | | |
| RSAD2, IFI27, OAS1, IFI44L, | 0.876 | 0.867 | 0.886 | 0.928 | 0.881 | 0.975 | 0.926 | 0.839 | 1 |
| SLC1A2, IL1R2, FAM20A, MMP8 | | | | | | | | | |
| IFI44L, SIGLEC1, ISG15, HERC6, | 0.876 | 0.867 | 0.886 | 0.925 | 0.877 | 0.973 | 0.959 | 0.893 | 1 |
| SLC1A2, IL1R2, FAM20A, MMP8 | | | | | | | | | |
| RSAD2, IFIT1, SIGLEC1, ISG15, | 0.876 | 0.867 | 0.885 | 0.921 | 0.864 | 0.978 | 0.967 | 0.916 | 1 |
| IL1R2, FAM20A, RETN, MMP8 | | | | | | | | | |
| RSAD2, IFI27, OAS1, IFIT1, | 0.876 | 0.867 | 0.886 | 0.921 | 0.862 | 0.980 | 0.951 | 0.893 | 1 |
| IFI44L, OLAH, RETN, MMP8 | | | | | | | | | |
| RSAD2, IFI27, OAS1, SIGLEC1, | 0.876 | 0.866 | 0.887 | 0.924 | 0.867 | 0.982 | 0.946 | 0.874 | 1 |
| SLC1A2, FAM20A, RETN, MMP8 | | | | | | | | | |
| RSAD2, IFI27, OAS1, IFIT1, | 0.876 | 0.867 | 0.885 | 0.919 | 0.862 | 0.975 | 0.941 | 0.885 | 0.998 |
| SIGLEC1, ISG15, OLAH, MMP8 | | | | | | | | | |
| RSAD2, IFI27, OAS1, IFIT1, | 0.876 | 0.868 | 0.885 | 0.917 | 0.862 | 0.973 | 0.957 | 0.907 | 1 |
| IFI44L, SIGLEC1, IL1R2, RETN | | | | | | | | | |
| RSAD2, IFI27, IFI44L, SIGLEC1, | 0.876 | 0.867 | 0.886 | 0.927 | 0.877 | 0.976 | 0.946 | 0.893 | 0.998 |
| HERC6, SLC1A2, IL1R2, OLAH | | | | | | | | | |
| IFI27, OAS1, IFI44L, SIGLEC1, | 0.876 | 0.867 | 0.886 | 0.928 | 0.879 | 0.976 | 0.941 | 0.884 | 0.998 |
| HERC6, SLC1A2, IL1R2, OLAH | | | | | | | | | |
| RSAD2, IFI27, IFIT1, HERC6, | 0.876 | 0.867 | 0.886 | 0.925 | 0.874 | 0.976 | 0.935 | 0.870 | 1 |
| SLC1A2, IL1R2, OLAH, RETN | | | | | | | | | |
| RSAD2, IFI27, OAS1, ISG15, | 0.876 | 0.867 | 0.886 | 0.922 | 0.866 | 0.978 | 0.951 | 0.897 | 1 |
| IL1R2, OLAH, RETN, MMP8 | | | | | | | | | |
| IFI27, IFIT1, IFI44L, SIGLEC1, | 0.876 | 0.867 | 0.886 | 0.927 | 0.877 | 0.978 | 0.942 | 0.887 | 0.998 |
| HERC6, SLC1A2, OLAH, MMP8 | | | | | | | | | |
| RSAD2, IFI27, IFIT1, ISG15, | 0.876 | 0.867 | 0.885 | 0.928 | 0.880 | 0.976 | 0.926 | 0.840 | 1 |
| SLC1A2, IL1R2, FAM20A, MMP8 | | | | | | | | | |
| RSAD2, IFI27, IFIT1, IFI44L, | 0.876 | 0.867 | 0.886 | 0.914 | 0.849 | 0.979 | 0.945 | 0.876 | 1 |
| SIGLEC1, ISG15, FAM20A, MMP8 | | | | | | | | | |
| RSAD2, IFI27, OAS1, IFIT1, | 0.876 | 0.867 | 0.885 | 0.921 | 0.867 | 0.975 | 0.965 | 0.920 | 1 |
| SIGLEC1, IL1R2, RETN, MMP8 | | | | | | | | | |
| IFI27, IFI44L, SIGLEC1, HERC6, | 0.876 | 0.866 | 0.887 | 0.927 | 0.872 | 0.982 | 0.948 | 0.871 | 1 |
| SLC1A2, FAM20A, RETN, MMP8 | | | | | | | | | |
| SIGLEC1, ISG15, HERC6, SLC1A2, | 0.876 | 0.867 | 0.886 | 0.924 | 0.876 | 0.973 | 0.959 | 0.895 | 1 |
| IL1R2, OLAH, FAM20A, MMP8 | | | | | | | | | |
| RSAD2, IFI27, IFI44L, SIGLEC1, | 0.876 | 0.868 | 0.885 | 0.927 | 0.877 | 0.977 | 0.950 | 0.899 | 1 |
| SLC1A2, IL1R2, OLAH, MMP8 | | | | | | | | | |
| RSAD2, IFI27, IFIT1, SIGLEC1, | 0.876 | 0.868 | 0.885 | 0.923 | 0.872 | 0.974 | 0.945 | 0.894 | 0.995 |
| ISG15, IL1R2, OLAH, MMP8 | | | | | | | | | |
| IFI27, OAS1, IFI44L, SIGLEC1, | 0.876 | 0.867 | 0.885 | 0.928 | 0.878 | 0.978 | 0.942 | 0.885 | 0.999 |
| ISG15, SLC1A2, IL1R2, OLAH | | | | | | | | | |
| RSAD2, IFI27, OAS1, SIGLEC1, | 0.876 | 0.867 | 0.885 | 0.929 | 0.876 | 0.981 | 0.947 | 0.883 | 1 |
| ISG15, SLC1A2, OLAH, RETN | | | | | | | | | |
| RSAD2, IFI27, IFI44L, SIGLEC1, | 0.876 | 0.868 | 0.885 | 0.929 | 0.878 | 0.979 | 0.943 | 0.889 | 0.998 |
| ISG15, SLC1A2, IL1R2, OLAH | | | | | | | | | |
| OAS1, IFIT1, SIGLEC1, ISG15, | 0.876 | 0.867 | 0.886 | 0.915 | 0.855 | 0.975 | 0.947 | 0.889 | 1 |
| HERC6, OLAH, FAM20A, RETN | | | | | | | | | |
| RSAD2, IFI27, IFIT1, IFI44L, | 0.876 | 0.868 | 0.885 | 0.920 | 0.863 | 0.978 | 0.948 | 0.891 | 1 |
| SIGLEC1, ISG15, OLAH, RETN | | | | | | | | | |
| IFI27, IFI44L, SIGLEC1, ISG15, | 0.876 | 0.866 | 0.886 | 0.923 | 0.868 | 0.979 | 0.942 | 0.863 | 1 |
| HERC6, SLC1A2, FAM20A, RETN | | | | | | | | | |
| RSAD2, IFI27, ISG15, HERC6, | 0.876 | 0.867 | 0.885 | 0.926 | 0.876 | 0.977 | 0.933 | 0.867 | 0.998 |
| SLC1A2, IL1R2, OLAH, RETN | | | | | | | | | |
| IFI27, IFIT1, IFI44L, SIGLEC1, | 0.876 | 0.867 | 0.885 | 0.926 | 0.875 | 0.977 | 0.950 | 0.894 | 1 |
| SLC1A2, IL1R2, OLAH, RETN | | | | | | | | | |
| RSAD2, IFI27, OAS1, IFIT1, ISG15, | 0.876 | 0.867 | 0.886 | 0.918 | 0.858 | 0.978 | 0.932 | 0.861 | 1 |
| HERC6, RETN, MMP8 | | | | | | | | | |
| RSAD2, IFI27, OAS1, IFIT1, | 0.876 | 0.867 | 0.885 | 0.926 | 0.877 | 0.975 | 0.927 | 0.843 | 1 |
| SLC1A2, IL1R2, FAM20A, MMP8 | | | | | | | | | |
| RSAD2, IFI27, OAS1, IFIT1, | 0.876 | 0.867 | 0.885 | 0.928 | 0.876 | 0.980 | 0.950 | 0.888 | 1 |
| SIGLEC1, SLC1A2, OLAH, RETN | | | | | | | | | |
| IFI27, IFIT1, ISG15, SLC1A2, | 0.876 | 0.867 | 0.886 | 0.933 | 0.884 | 0.982 | 0.930 | 0.848 | 1 |
| IL1R2, FAM20A, RETN, MMP8 | | | | | | | | | |
| OAS1, SIGLEC1, HERC6, IL1R2, | 0.876 | 0.866 | 0.886 | 0.920 | 0.863 | 0.977 | 0.964 | 0.912 | 1 |
| OLAH, FAM20A, RETN, MMP8 | | | | | | | | | |
| RSAD2, IFI27, IFI44L, SIGLEC1, | 0.876 | 0.867 | 0.885 | 0.926 | 0.875 | 0.977 | 0.949 | 0.892 | 1 |
| SLC1A2, IL1R2, OLAH, RETN | | | | | | | | | |
| IFI27, OAS1, IFIT1, IFI44L, | 0.876 | 0.868 | 0.885 | 0.922 | 0.870 | 0.975 | 0.948 | 0.896 | 1.000 |
| SIGLEC1, ISG15, IL1R2, RETN | | | | | | | | | |
| OAS1, IFIT1, SIGLEC1, ISG15, | 0.876 | 0.866 | 0.886 | 0.922 | 0.868 | 0.975 | 0.952 | 0.891 | 1 |
| SLC1A2, OLAH, FAM20A, MMP8 | | | | | | | | | |
| RSAD2, OAS1, SIGLEC1, IL1R2, | 0.876 | 0.866 | 0.886 | 0.920 | 0.863 | 0.977 | 0.962 | 0.906 | 1 |
| OLAH, FAM20A, RETN, MMP8 | | | | | | | | | |
| RSAD2, IFI27, OAS1, IFIT1, ISG15, | 0.876 | 0.867 | 0.886 | 0.927 | 0.876 | 0.978 | 0.933 | 0.866 | 0.999 |
| HERC6, SLC1A2, OLAH | | | | | | | | | |
| RSAD2, IFI27, IFI44L, SIGLEC1, | 0.876 | 0.866 | 0.886 | 0.918 | 0.858 | 0.977 | 0.941 | 0.869 | 1 |
| ISG15, HERC6, FAM20A, MMP8 | | | | | | | | | |
| IFI27, IFIT1, IFI44L, HERC6, | 0.876 | 0.867 | 0.886 | 0.930 | 0.881 | 0.979 | 0.927 | 0.844 | 1 |
| SLC1A2, IL1R2, FAM20A, RETN | | | | | | | | | |
| IFI27, OAS1, IFIT1, SIGLEC1, | 0.876 | 0.867 | 0.885 | 0.923 | 0.870 | 0.976 | 0.963 | 0.918 | 1 |
| ISG15, IL1R2, RETN, MMP8 | | | | | | | | | |
| IFI27, OAS1, IFIT1, IFI44L, | 0.876 | 0.866 | 0.886 | 0.929 | 0.879 | 0.978 | 0.934 | 0.868 | 0.999 |
| HERC6, SLC1A2, OLAH, MMP8 | | | | | | | | | |
| RSAD2, IFI27, OAS1, IFI44L, | 0.876 | 0.867 | 0.885 | 0.926 | 0.873 | 0.979 | 0.951 | 0.889 | 1 |
| SIGLEC1, SLC1A2, OLAH, RETN | | | | | | | | | |
| RSAD2, IFI27, IFI44L, ISG15, | 0.876 | 0.867 | 0.885 | 0.927 | 0.876 | 0.977 | 0.935 | 0.870 | 0.999 |
| HERC6, SLC1A2, OLAH, RETN | | | | | | | | | |
| IFI27, OAS1, IFIT1, IFI44L, ISG15, | 0.876 | 0.867 | 0.885 | 0.921 | 0.869 | 0.973 | 0.940 | 0.886 | 0.995 |
| IL1R2, OLAH, MMP8 | | | | | | | | | |
| RSAD2, SIGLEC1, ISG15, SLC1A2, | 0.876 | 0.866 | 0.886 | 0.924 | 0.877 | 0.972 | 0.961 | 0.901 | 1 |
| IL1R2, OLAH, FAM20A, MMP8 | | | | | | | | | |
| OAS1, IFI44L, SIGLEC1, ISG15, | 0.876 | 0.866 | 0.886 | 0.916 | 0.858 | 0.974 | 0.963 | 0.908 | 1 |
| IL1R2, OLAH, FAM20A, MMP8 | | | | | | | | | |
| RSAD2, IFI27, OAS1, SIGLEC1, | 0.876 | 0.867 | 0.885 | 0.923 | 0.869 | 0.978 | 0.949 | 0.887 | 1 |
| HERC6, SLC1A2, RETN, MMP8 | | | | | | | | | |
| RSAD2, IFI27, OAS1, IFIT1, ISG15, | 0.876 | 0.867 | 0.885 | 0.922 | 0.871 | 0.973 | 0.946 | 0.894 | 0.998 |
| IL1R2, OLAH, MMP8 | | | | | | | | | |
| IFI27, IFIT1, ISG15, SLC1A2, | 0.876 | 0.866 | 0.886 | 0.926 | 0.873 | 0.978 | 0.940 | 0.879 | 1 |
| IL1R2, OLAH, RETN, MMP8 | | | | | | | | | |
| IFI27, OAS1, IFIT1, IFI44L, | 0.876 | 0.867 | 0.885 | 0.922 | 0.866 | 0.979 | 0.940 | 0.867 | 1 |
| SIGLEC1, ISG15, SLC1A2, FAM20A | | | | | | | | | |
| IFI27, OAS1, IFI44L, SIGLEC1, | 0.876 | 0.867 | 0.885 | 0.927 | 0.878 | 0.976 | 0.951 | 0.900 | 1 |
| SLC1A2, IL1R2, OLAH, MMP8 | | | | | | | | | |
| RSAD2, IFI27, OAS1, IFIT1, | 0.876 | 0.866 | 0.885 | 0.931 | 0.886 | 0.977 | 0.925 | 0.839 | 1 |
| HERC6, SLC1A2, IL1R2, FAM20A | | | | | | | | | |
| RSAD2, IFI27, IFIT1, IFI44L, | 0.876 | 0.867 | 0.885 | 0.925 | 0.872 | 0.977 | 0.961 | 0.920 | 1 |
| SIGLEC1, IL1R2, RETN, MMP8 | | | | | | | | | |
| OAS1, IFIT1, IFI44L, SIGLEC1, | 0.876 | 0.866 | 0.886 | 0.916 | 0.857 | 0.975 | 0.946 | 0.888 | 1 |
| ISG15, HERC6, OLAH, FAM20A | | | | | | | | | |
| IFI27, OAS1, IFI44L, SIGLEC1, | 0.876 | 0.867 | 0.885 | 0.926 | 0.875 | 0.978 | 0.952 | 0.894 | 1 |
| SLC1A2, IL1R2, OLAH, RETN | | | | | | | | | |
| IFIT1, IFI44L, SIGLEC1, HERC6, | 0.876 | 0.866 | 0.886 | 0.926 | 0.876 | 0.975 | 0.938 | 0.868 | 1 |
| SLC1A2, IL1R2, OLAH, FAM20A | | | | | | | | | |
| RSAD2, IFI27, OAS1, IFIT1, ISG15, | 0.876 | 0.867 | 0.885 | 0.926 | 0.873 | 0.978 | 0.933 | 0.866 | 0.999 |
| SLC1A2, OLAH, MMP8 | | | | | | | | | |
| IFI27, IFI44L, ISG15, HERC6, | 0.876 | 0.866 | 0.886 | 0.931 | 0.882 | 0.979 | 0.926 | 0.842 | 1 |
| SLC1A2, IL1R2, FAM20A, RETN | | | | | | | | | |
| RSAD2, IFI27, IFIT1, IFI44L, | 0.876 | 0.867 | 0.885 | 0.926 | 0.876 | 0.977 | 0.941 | 0.885 | 0.997 |
| SIGLEC1, HERC6, SLC1A2, OLAH | | | | | | | | | |
| RSAD2, IFI27, IFIT1, SIGLEC1, | 0.876 | 0.867 | 0.885 | 0.929 | 0.878 | 0.980 | 0.939 | 0.878 | 1 |
| ISG15, SLC1A2, OLAH, RETN | | | | | | | | | |
| RSAD2, IFI27, OAS1, IFIT1, | 0.876 | 0.866 | 0.885 | 0.925 | 0.869 | 0.981 | 0.941 | 0.869 | 1 |
| SIGLEC1, SLC1A2, FAM20A, RETN | | | | | | | | | |
| RSAD2, IFI27, IFIT1, IFI44L, | 0.876 | 0.867 | 0.885 | 0.926 | 0.875 | 0.977 | 0.935 | 0.870 | 0.999 |
| HERC6, SLC1A2, OLAH, RETN | | | | | | | | | |
| RSAD2, IFI44L, SIGLEC1, ISG15, | 0.876 | 0.866 | 0.885 | 0.917 | 0.860 | 0.974 | 0.962 | 0.906 | 1 |
| IL1R2, OLAH, FAM20A, MMP8 | | | | | | | | | |
| OAS1, SIGLEC1, ISG15, HERC6, | 0.876 | 0.866 | 0.886 | 0.924 | 0.876 | 0.972 | 0.958 | 0.892 | 1 |
| SLC1A2, IL1R2, FAM20A, MMP8 | | | | | | | | | |
| RSAD2, IFI27, IFIT1, IFI44L, | 0.876 | 0.867 | 0.885 | 0.920 | 0.863 | 0.976 | 0.939 | 0.882 | 0.997 |
| SIGLEC1, ISG15, OLAH, MMP8 | | | | | | | | | |
| RSAD2, IFI27, IFIT1, IFI44L, | 0.876 | 0.867 | 0.885 | 0.921 | 0.867 | 0.975 | 0.941 | 0.883 | 1.000 |
| HERC6, IL1R2, RETN, MMP8 | | | | | | | | | |
| RSAD2, IFIT1, IFI44L, SIGLEC1, | 0.876 | 0.867 | 0.885 | 0.927 | 0.880 | 0.974 | 0.962 | 0.902 | 1 |
| SLC1A2, IL1R2, FAM20A, MMP8 | | | | | | | | | |
| RSAD2, IFI27, IFI44L, SLC1A2, | 0.876 | 0.866 | 0.885 | 0.930 | 0.880 | 0.980 | 0.924 | 0.836 | 1 |
| IL1R2, FAM20A, RETN, MMP8 | | | | | | | | | |
| RSAD2, IFI27, IFI44L, HERC6, | 0.876 | 0.866 | 0.885 | 0.925 | 0.874 | 0.976 | 0.935 | 0.870 | 0.999 |
| SLC1A2, IL1R2, OLAH, RETN | | | | | | | | | |
| RSAD2, IFI27, OAS1, IFI44L, | 0.876 | 0.867 | 0.885 | 0.920 | 0.865 | 0.975 | 0.962 | 0.916 | 1 |
| SIGLEC1, IL1R2, RETN, MMP8 | | | | | | | | | |
| RSAD2, OAS1, IFIT1, SIGLEC1, | 0.876 | 0.867 | 0.885 | 0.919 | 0.860 | 0.977 | 0.952 | 0.895 | 1 |
| HERC6, IL1R2, FAM20A, RETN | | | | | | | | | |
| RSAD2, IFI27, IFI44L, HERC6, | 0.876 | 0.866 | 0.886 | 0.930 | 0.882 | 0.979 | 0.926 | 0.842 | 1 |
| SLC1A2, IL1R2, FAM20A, RETN | | | | | | | | | |
| RSAD2, IFI27, OAS1, IFIT1, | 0.876 | 0.867 | 0.885 | 0.918 | 0.860 | 0.976 | 0.948 | 0.889 | 1 |
| SIGLEC1, ISG15, OLAH, RETN | | | | | | | | | |
| IFIT1, IFI44L, SIGLEC1, SLC1A2, | 0.876 | 0.866 | 0.885 | 0.929 | 0.882 | 0.975 | 0.961 | 0.900 | 1 |
| IL1R2, FAM20A, RETN, MMP8 | | | | | | | | | |
| RSAD2, IFI27, IFIT1, ISG15, | 0.876 | 0.867 | 0.885 | 0.927 | 0.878 | 0.976 | 0.932 | 0.868 | 0.995 |
| SLC1A2, IL1R2, OLAH, MMP8 | | | | | | | | | |
| RSAD2, IFI27, OAS1, IFI44L, | 0.876 | 0.866 | 0.885 | 0.928 | 0.878 | 0.978 | 0.932 | 0.864 | 0.999 |
| HERC6, SLC1A2, OLAH, MMP8 | | | | | | | | | |
| RSAD2, IFI27, OAS1, IFI44L, | 0.876 | 0.867 | 0.885 | 0.927 | 0.876 | 0.977 | 0.948 | 0.890 | 1 |
| SIGLEC1, SLC1A2, IL1R2, OLAH | | | | | | | | | |
| RSAD2, IFI27, IFIT1, IFI44L, | 0.876 | 0.867 | 0.884 | 0.926 | 0.874 | 0.978 | 0.950 | 0.894 | 1 |
| SIGLEC1, SLC1A2, OLAH, RETN | | | | | | | | | |
| RSAD2, IFI27, IFIT1, SIGLEC1, | 0.876 | 0.867 | 0.884 | 0.919 | 0.863 | 0.975 | 0.947 | 0.891 | 1 |
| ISG15, IL1R2, OLAH, RETN | | | | | | | | | |
| OAS1, IFI44L, SIGLEC1, HERC6, | 0.876 | 0.866 | 0.886 | 0.923 | 0.875 | 0.971 | 0.959 | 0.893 | 1 |
| SLC1A2, IL1R2, FAM20A, MMP8 | | | | | | | | | |
| RSAD2, IFI27, IFIT1, SLC1A2, | 0.876 | 0.866 | 0.885 | 0.925 | 0.873 | 0.977 | 0.943 | 0.883 | 1 |
| IL1R2, OLAH, RETN, MMP8 | | | | | | | | | |
| RSAD2, IFI27, OAS1, SIGLEC1, | 0.876 | 0.867 | 0.885 | 0.921 | 0.866 | 0.977 | 0.947 | 0.884 | 1 |
| ISG15, HERC6, SLC1A2, RETN | | | | | | | | | |
| RSAD2, IFI27, IFIT1, IFI44L, | 0.876 | 0.867 | 0.885 | 0.915 | 0.856 | 0.975 | 0.939 | 0.883 | 0.995 |
| SIGLEC1, HERC6, RETN, MMP8 | | | | | | | | | |
| RSAD2, IFI27, IFIT1, ISG15, IL1R2, | 0.876 | 0.866 | 0.885 | 0.921 | 0.865 | 0.977 | 0.950 | 0.897 | 1 |
| OLAH, RETN, MMP8 | | | | | | | | | |
| RSAD2, IFI27, OAS1, IFI44L, | 0.876 | 0.867 | 0.884 | 0.918 | 0.862 | 0.974 | 0.952 | 0.900 | 1 |
| SIGLEC1, ISG15, IL1R2, RETN | | | | | | | | | |
| RSAD2, OAS1, IFIT1, SIGLEC1, | 0.876 | 0.866 | 0.885 | 0.918 | 0.859 | 0.978 | 0.941 | 0.873 | 1 |
| HERC6, FAM20A, RETN, MMP8 | | | | | | | | | |
| RSAD2, OAS1, IFIT1, SIGLEC1, | 0.876 | 0.866 | 0.885 | 0.928 | 0.882 | 0.974 | 0.962 | 0.901 | 1 |
| SLC1A2, IL1R2, FAM20A, MMP8 | | | | | | | | | |
| RSAD2, IFI27, OAS1, IFIT1, | 0.876 | 0.867 | 0.884 | 0.922 | 0.875 | 0.969 | 0.952 | 0.907 | 0.997 |
| SIGLEC1, ISG15, HERC6, IL1R2 | | | | | | | | | |
| RSAD2, IFI27, OAS1, IFI44L, | 0.876 | 0.867 | 0.885 | 0.926 | 0.874 | 0.978 | 0.954 | 0.902 | 1 |
| SIGLEC1, SLC1A2, OLAH, MMP8 | | | | | | | | | |
| RSAD2, IFI27, IFIT1, IFI44L, | 0.876 | 0.867 | 0.884 | 0.919 | 0.870 | 0.969 | 0.940 | 0.888 | 0.992 |
| SIGLEC1, ISG15, HERC6, IL1R2 | | | | | | | | | |
| RSAD2, IFI44L, SIGLEC1, | 0.876 | 0.866 | 0.885 | 0.924 | 0.875 | 0.973 | 0.959 | 0.898 | 1 |
| SLC1A2, IL1R2, OLAH, FAM20A, MMP8 | | | | | | | | | |
| IFI27, OAS1, IFI44L, SIGLEC1, | 0.876 | 0.865 | 0.886 | 0.914 | 0.854 | 0.973 | 0.945 | 0.871 | 1 |
| ISG15, HERC6, FAM20A, MMP8 | | | | | | | | | |
| RSAD2, IFIT1, IFI44L, SIGLEC1, | 0.876 | 0.866 | 0.885 | 0.925 | 0.875 | 0.975 | 0.927 | 0.853 | 1 |
| HERC6, SLC1A2, OLAH, FAM20A | | | | | | | | | |
| RSAD2, IFI27, IFI44L, ISG15, | 0.876 | 0.866 | 0.885 | 0.922 | 0.867 | 0.978 | 0.952 | 0.898 | 1 |
| IL1R2, OLAH, RETN, MMP8 | | | | | | | | | |
| RSAD2, OAS1, IFIT1, SIGLEC1, | 0.876 | 0.866 | 0.885 | 0.917 | 0.858 | 0.976 | 0.943 | 0.879 | 1 |
| ISG15, HERC6, FAM20A, RETN | | | | | | | | | |
| RSAD2, IFI27, IFIT1, ISG15, | 0.875 | 0.866 | 0.885 | 0.926 | 0.877 | 0.976 | 0.926 | 0.858 | 0.994 |
| HERC6, SLC1A2, IL1R2, OLAH | | | | | | | | | |
| RSAD2, IFIT1, SIGLEC1, ISG15, | 0.875 | 0.866 | 0.885 | 0.927 | 0.880 | 0.974 | 0.960 | 0.894 | 1 |
| SLC1A2, IL1R2, FAM20A, MMP8 | | | | | | | | | |
| IFI27, IFI44L, ISG15, HERC6, | 0.875 | 0.866 | 0.885 | 0.926 | 0.875 | 0.977 | 0.936 | 0.871 | 1 |
| SLC1A2, IL1R2, OLAH, RETN | | | | | | | | | |
| RSAD2, IFI27, IFI44L, ISG15, | 0.875 | 0.867 | 0.884 | 0.923 | 0.870 | 0.976 | 0.937 | 0.876 | 0.998 |
| HERC6, IL1R2, RETN, MMP8 | | | | | | | | | |
| IFI27, OAS1, IFI44L, SLC1A2, | 0.875 | 0.866 | 0.885 | 0.930 | 0.881 | 0.979 | 0.922 | 0.833 | 1 |
| IL1R2, FAM20A, RETN, MMP8 | | | | | | | | | |
| RSAD2, IFI27, OAS1, IFI44L, | 0.875 | 0.867 | 0.884 | 0.923 | 0.871 | 0.974 | 0.939 | 0.883 | 0.996 |
| ISG15, IL1R2, OLAH, MMP8 | | | | | | | | | |
| RSAD2, IFI27, OAS1, SIGLEC1, | 0.875 | 0.867 | 0.884 | 0.922 | 0.875 | 0.968 | 0.951 | 0.905 | 0.997 |
| ISG15, HERC6, IL1R2, MMP8 | | | | | | | | | |
| IFI27, OAS1, IFI44L, SIGLEC1, | 0.875 | 0.866 | 0.885 | 0.923 | 0.867 | 0.980 | 0.940 | 0.864 | 1 |
| ISG15, SLC1A2, FAM20A, RETN | | | | | | | | | |
| RSAD2, IFI27, IFI44L, SIGLEC1, | 0.875 | 0.868 | 0.883 | 0.920 | 0.870 | 0.970 | 0.940 | 0.888 | 0.992 |
| ISG15, HERC6, IL1R2, MMP8 | | | | | | | | | |
| OAS1, SIGLEC1, ISG15, IL1R2, | 0.875 | 0.866 | 0.885 | 0.914 | 0.854 | 0.974 | 0.966 | 0.914 | 1 |
| OLAH, FAM20A, RETN, MMP8 | | | | | | | | | |
| OAS1, IFIT1, IFI44L, SIGLEC1, | 0.875 | 0.866 | 0.885 | 0.913 | 0.850 | 0.976 | 0.959 | 0.907 | 1 |
| ISG15, IL1R2, OLAH, FAM20A | | | | | | | | | |
| RSAD2, IFI27, IFIT1, HERC6, | 0.875 | 0.866 | 0.885 | 0.933 | 0.885 | 0.981 | 0.927 | 0.845 | 1 |
| SLC1A2, IL1R2, FAM20A, RETN | | | | | | | | | |
| RSAD2, IFI27, IFIT1, ISG15, | 0.875 | 0.866 | 0.885 | 0.927 | 0.876 | 0.978 | 0.933 | 0.866 | 0.999 |
| HERC6, SLC1A2, OLAH, RETN | | | | | | | | | |
| IFI27, IFIT1, ISG15, HERC6, | 0.875 | 0.866 | 0.885 | 0.926 | 0.875 | 0.978 | 0.934 | 0.868 | 0.999 |
| SLC1A2, IL1R2, OLAH, RETN | | | | | | | | | |
| IFI27, OAS1, IFIT1, IFI44L, | 0.875 | 0.866 | 0.884 | 0.927 | 0.877 | 0.977 | 0.949 | 0.895 | 1 |
| SIGLEC1, SLC1A2, IL1R2, OLAH | | | | | | | | | |
| RSAD2, OAS1, IFIT1, IFI44L, | 0.875 | 0.865 | 0.885 | 0.911 | 0.846 | 0.975 | 0.954 | 0.903 | 1 |
| SIGLEC1, OLAH, FAM20A, MMP8 | | | | | | | | | |
| IFI27, IFIT1, ISG15, HERC6, | 0.875 | 0.866 | 0.885 | 0.929 | 0.882 | 0.976 | 0.928 | 0.842 | 1 |
| SLC1A2, IL1R2, FAM20A, MMP8 | | | | | | | | | |
| RSAD2, OAS1, SIGLEC1, ISG15, | 0.875 | 0.866 | 0.885 | 0.919 | 0.861 | 0.976 | 0.961 | 0.902 | 1 |
| IL1R2, FAM20A, RETN, MMP8 | | | | | | | | | |
| RSAD2, IFI44L, SIGLEC1, HERC6, | 0.875 | 0.866 | 0.885 | 0.925 | 0.877 | 0.972 | 0.959 | 0.895 | 1 |
| SLC1A2, IL1R2, FAM20A, MMP8 | | | | | | | | | |
| RSAD2, OAS1, IFIT1, SIGLEC1, | 0.875 | 0.866 | 0.885 | 0.923 | 0.869 | 0.976 | 0.952 | 0.892 | 1 |
| SLC1A2, OLAH, FAM20A, MMP8 | | | | | | | | | |
| IFI27, IFIT1, IFI44L, ISG15, | 0.875 | 0.866 | 0.885 | 0.929 | 0.882 | 0.976 | 0.927 | 0.842 | 1 |
| HERC6, SLC1A2, IL1R2, FAM20A | | | | | | | | | |
| RSAD2, IFI27, IFIT1, IFI44L, | 0.875 | 0.867 | 0.884 | 0.919 | 0.866 | 0.972 | 0.942 | 0.889 | 0.995 |
| ISG15, IL1R2, OLAH, MMP8 | | | | | | | | | |
| RSAD2, IFIT1, SIGLEC1, ISG15, | 0.875 | 0.866 | 0.884 | 0.918 | 0.860 | 0.977 | 0.945 | 0.877 | 1 |
| HERC6, FAM20A, RETN, MMP8 | | | | | | | | | |
| IFI27, OAS1, IFI44L, SIGLEC1, | 0.875 | 0.865 | 0.886 | 0.923 | 0.866 | 0.980 | 0.942 | 0.868 | 1 |
| SLC1A2, FAM20A, RETN, MMP8 | | | | | | | | | |
| RSAD2, IFIT1, IFI44L, SIGLEC1, | 0.875 | 0.865 | 0.885 | 0.916 | 0.857 | 0.975 | 0.947 | 0.890 | 1 |
| ISG15, HERC6, OLAH, FAM20A | | | | | | | | | |
| RSAD2, IFI27, IFIT1, IFI44L, | 0.875 | 0.867 | 0.884 | 0.926 | 0.875 | 0.978 | 0.948 | 0.894 | 1 |
| SIGLEC1, SLC1A2, OLAH, MMP8 | | | | | | | | | |
| OAS1, IFIT1, IFI44L, SIGLEC1, | 0.875 | 0.866 | 0.885 | 0.927 | 0.881 | 0.974 | 0.940 | 0.869 | 1 |
| HERC6, SLC1A2, IL1R2, FAM20A | | | | | | | | | |
| RSAD2, IFI27, IFIT1, SIGLEC1, | 0.875 | 0.866 | 0.884 | 0.918 | 0.860 | 0.975 | 0.949 | 0.900 | 0.998 |
| ISG15, HERC6, RETN, MMP8 | | | | | | | | | |
| RSAD2, IFI27, OAS1, IFIT1, | 0.875 | 0.866 | 0.885 | 0.922 | 0.866 | 0.979 | 0.941 | 0.868 | 1 |
| IFI44L, SIGLEC1, SLC1A2, FAM20A | | | | | | | | | |
| OAS1, IFIT1, IFI44L, SIGLEC1, | 0.875 | 0.865 | 0.886 | 0.912 | 0.847 | 0.977 | 0.954 | 0.902 | 1 |
| ISG15, OLAH, FAM20A, MMP8 | | | | | | | | | |
| RSAD2, OAS1, IFIT1, SIGLEC1, | 0.875 | 0.865 | 0.885 | 0.914 | 0.853 | 0.974 | 0.950 | 0.895 | 1 |
| ISG15, HERC6, OLAH, FAM20A | | | | | | | | | |
| RSAD2, IFI27, IFIT1, IFI44L, | 0.875 | 0.867 | 0.884 | 0.921 | 0.869 | 0.974 | 0.936 | 0.876 | 0.995 |
| ISG15, HERC6, IL1R2, RETN | | | | | | | | | |
| IFI27, OAS1, IFIT1, IFI44L, | 0.875 | 0.866 | 0.885 | 0.921 | 0.864 | 0.979 | 0.939 | 0.865 | 1 |
| SIGLEC1, SLC1A2, FAM20A, MMP8 | | | | | | | | | |
| RSAD2, IFIT1, SIGLEC1, HERC6, | 0.875 | 0.866 | 0.885 | 0.918 | 0.860 | 0.977 | 0.952 | 0.897 | 1 |
| IL1R2, OLAH, FAM20A, RETN | | | | | | | | | |
| IFI27, OAS1, IFIT1, ISG15, | 0.875 | 0.866 | 0.884 | 0.927 | 0.878 | 0.976 | 0.933 | 0.868 | 0.997 |
| SLC1A2, IL1R2, OLAH, MMP8 | | | | | | | | | |
| RSAD2, IFI27, IFI44L, SIGLEC1, | 0.875 | 0.865 | 0.885 | 0.926 | 0.871 | 0.982 | 0.949 | 0.877 | 1 |
| SLC1A2, FAM20A, RETN, MMP8 | | | | | | | | | |
| RSAD2, IFIT1, SIGLEC1, ISG15, | 0.875 | 0.866 | 0.884 | 0.918 | 0.860 | 0.976 | 0.954 | 0.898 | 1 |
| HERC6, IL1R2, FAM20A, RETN | | | | | | | | | |
| IFIT1, SIGLEC1, SLC1A2, IL1R2, | 0.875 | 0.866 | 0.885 | 0.927 | 0.878 | 0.975 | 0.961 | 0.903 | 1 |
| OLAH, FAM20A, RETN, MMP8 | | | | | | | | | |
| IFI27, OAS1, IFIT1, IFI44L, | 0.875 | 0.865 | 0.885 | 0.922 | 0.866 | 0.978 | 0.934 | 0.852 | 1 |
| HERC6, SLC1A2, FAM20A, RETN | | | | | | | | | |
| RSAD2, IFI44L, SIGLEC1, HERC6, | 0.875 | 0.866 | 0.885 | 0.921 | 0.865 | 0.977 | 0.959 | 0.897 | 1 |
| IL1R2, FAM20A, RETN, MMP8 | | | | | | | | | |
| RSAD2, OAS1, IFIT1, IFI44L, | 0.875 | 0.866 | 0.884 | 0.913 | 0.849 | 0.976 | 0.962 | 0.913 | 1 |
| SIGLEC1, IL1R2, OLAH, FAM20A | | | | | | | | | |
| IFI27, OAS1, IFI44L, ISG15, | 0.875 | 0.865 | 0.885 | 0.929 | 0.880 | 0.978 | 0.924 | 0.854 | 0.994 |
| HERC6, SLC1A2, OLAH, MMP8 | | | | | | | | | |
| RSAD2, IFI27, IFIT1, IFI44L, | 0.875 | 0.867 | 0.884 | 0.931 | 0.881 | 0.981 | 0.942 | 0.884 | 1 |
| SIGLEC1, ISG15, SLC1A2, OLAH | | | | | | | | | |
| OAS1, IFIT1, SIGLEC1, ISG15, | 0.875 | 0.866 | 0.885 | 0.927 | 0.881 | 0.973 | 0.959 | 0.893 | 1 |
| SLC1A2, IL1R2, FAM20A, MMP8 | | | | | | | | | |
| RSAD2, IFI27, SIGLEC1, HERC6, | 0.875 | 0.866 | 0.884 | 0.927 | 0.880 | 0.974 | 0.963 | 0.923 | 1 |
| SLC1A2, IL1R2, RETN, MMP8 | | | | | | | | | |
| IFIT1, IFI44L, SIGLEC1, ISG15, | 0.875 | 0.866 | 0.884 | 0.925 | 0.877 | 0.974 | 0.941 | 0.869 | 1 |
| HERC6, SLC1A2, IL1R2, FAM20A | | | | | | | | | |
| RSAD2, IFI27, OAS1, IFIT1, | 0.875 | 0.865 | 0.885 | 0.916 | 0.851 | 0.980 | 0.940 | 0.867 | 1 |
| SIGLEC1, ISG15, FAM20A, MMP8 | | | | | | | | | |
| IFI27, IFIT1, IFI44L, ISG15, | 0.875 | 0.866 | 0.884 | 0.926 | 0.874 | 0.977 | 0.935 | 0.870 | 1.000 |
| HERC6, SLC1A2, OLAH, RETN | | | | | | | | | |
| OAS1, SIGLEC1, ISG15, SLC1A2, | 0.875 | 0.866 | 0.885 | 0.923 | 0.875 | 0.972 | 0.961 | 0.901 | 1 |
| IL1R2, OLAH, FAM20A, MMP8 | | | | | | | | | |
| IFI44L, SIGLEC1, ISG15, SLC1A2, | 0.875 | 0.866 | 0.884 | 0.925 | 0.876 | 0.973 | 0.957 | 0.889 | 1 |
| IL1R2, FAM20A, RETN, MMP8 | | | | | | | | | |
| RSAD2, IFI27, OAS1, SLC1A2, | 0.875 | 0.866 | 0.885 | 0.929 | 0.877 | 0.980 | 0.939 | 0.873 | 1 |
| IL1R2, OLAH, RETN, MMP8 | | | | | | | | | |
| IFI27, OAS1, IFIT1, ISG15, | 0.875 | 0.865 | 0.885 | 0.927 | 0.879 | 0.975 | 0.926 | 0.841 | 1 |
| SLC1A2, IL1R2, FAM20A, MMP8 | | | | | | | | | |
| IFI27, OAS1, IFIT1, ISG15, | 0.875 | 0.865 | 0.885 | 0.926 | 0.872 | 0.979 | 0.941 | 0.877 | 1 |
| SLC1A2, OLAH, RETN, MMP8 | | | | | | | | | |
| IFI27, IFI44L, SIGLEC1, HERC6, | 0.875 | 0.866 | 0.884 | 0.927 | 0.879 | 0.975 | 0.955 | 0.909 | 1 |
| SLC1A2, IL1R2, RETN, MMP8 | | | | | | | | | |
| IFI27, OAS1, IFIT1, IFI44L, ISG15, | 0.875 | 0.865 | 0.885 | 0.928 | 0.878 | 0.978 | 0.928 | 0.862 | 0.995 |
| HERC6, SLC1A2, OLAH | | | | | | | | | |
| IFI27, OAS1, IFI44L, HERC6, | 0.875 | 0.865 | 0.885 | 0.928 | 0.878 | 0.979 | 0.932 | 0.864 | 0.999 |
| SLC1A2, IL1R2, OLAH, RETN | | | | | | | | | |
| IFI27, OAS1, IFI44L, HERC6, | 0.875 | 0.865 | 0.885 | 0.928 | 0.879 | 0.977 | 0.928 | 0.860 | 0.997 |
| SLC1A2, IL1R2, OLAH, MMP8 | | | | | | | | | |
| IFI27, OAS1, IFI44L, SIGLEC1, | 0.875 | 0.866 | 0.884 | 0.925 | 0.872 | 0.978 | 0.942 | 0.880 | 1 |
| HERC6, SLC1A2, RETN, MMP8 | | | | | | | | | |
| OAS1, IFIT1, SIGLEC1, HERC6, | 0.875 | 0.865 | 0.885 | 0.924 | 0.875 | 0.973 | 0.943 | 0.875 | 1 |
| SLC1A2, IL1R2, OLAH, FAM20A | | | | | | | | | |
| IFI27, IFIT1, SIGLEC1, HERC6, | 0.875 | 0.866 | 0.884 | 0.927 | 0.881 | 0.974 | 0.963 | 0.923 | 1 |
| SLC1A2, IL1R2, RETN, MMP8 | | | | | | | | | |
| IFI27, IFIT1, ISG15, HERC6, | 0.875 | 0.865 | 0.885 | 0.933 | 0.886 | 0.981 | 0.927 | 0.845 | 1 |
| SLC1A2, IL1R2, FAM20A, RETN | | | | | | | | | |
| RSAD2, IFI27, ISG15, SLC1A2, | 0.875 | 0.866 | 0.884 | 0.930 | 0.881 | 0.978 | 0.927 | 0.841 | 1 |
| IL1R2, FAM20A, RETN, MMP8 | | | | | | | | | |
| RSAD2, IFI27, IFI44L, HERC6, | 0.875 | 0.866 | 0.884 | 0.926 | 0.877 | 0.976 | 0.929 | 0.864 | 0.995 |
| SLC1A2, IL1R2, OLAH, MMP8 | | | | | | | | | |
| RSAD2, IFI27, OAS1, IFI44L, | 0.875 | 0.866 | 0.884 | 0.917 | 0.860 | 0.974 | 0.938 | 0.876 | 1 |
| ISG15, IL1R2, OLAH, RETN | | | | | | | | | |
| RSAD2, IFIT1, IFI44L, SIGLEC1, | 0.875 | 0.866 | 0.884 | 0.922 | 0.865 | 0.978 | 0.968 | 0.918 | 1 |
| IL1R2, FAM20A, RETN, MMP8 | | | | | | | | | |
| IFI27, IFIT1, IFI44L, ISG15, | 0.875 | 0.866 | 0.884 | 0.929 | 0.880 | 0.977 | 0.934 | 0.872 | 0.995 |
| SLC1A2, IL1R2, OLAH, MMP8 | | | | | | | | | |
| RSAD2, IFI27, OAS1, IFI44L, | 0.875 | 0.865 | 0.885 | 0.929 | 0.879 | 0.979 | 0.932 | 0.864 | 0.999 |
| ISG15, HERC6, SLC1A2, OLAH | | | | | | | | | |
| IFI27, OAS1, IFIT1, SIGLEC1, | 0.875 | 0.866 | 0.884 | 0.926 | 0.878 | 0.974 | 0.962 | 0.913 | 1 |
| SLC1A2, IL1R2, RETN, MMP8 | | | | | | | | | |
| IFIT1, SIGLEC1, ISG15, HERC6, | 0.875 | 0.865 | 0.885 | 0.924 | 0.875 | 0.974 | 0.945 | 0.873 | 1 |
| SLC1A2, IL1R2, OLAH, FAM20A | | | | | | | | | |
| RSAD2, IFI27, IFIT1, ISG15, | 0.875 | 0.865 | 0.884 | 0.930 | 0.883 | 0.977 | 0.925 | 0.840 | 1 |
| HERC6, SLC1A2, IL1R2, FAM20A | | | | | | | | | |
| RSAD2, IFI27, IFIT1, ISG15, | 0.875 | 0.865 | 0.885 | 0.927 | 0.877 | 0.978 | 0.928 | 0.862 | 0.995 |
| HERC6, SLC1A2, OLAH, MMP8 | | | | | | | | | |
| IFIT1, IFI44L, SIGLEC1, HERC6, | 0.875 | 0.865 | 0.885 | 0.925 | 0.874 | 0.976 | 0.933 | 0.861 | 1 |
| SLC1A2, OLAH, FAM20A, RETN | | | | | | | | | |
| RSAD2, IFI27, OAS1, ISG15, | 0.875 | 0.866 | 0.884 | 0.929 | 0.880 | 0.978 | 0.930 | 0.862 | 0.998 |
| SLC1A2, IL1R2, OLAH, MMP8 | | | | | | | | | |
| IFI27, OAS1, IFIT1, ISG15, | 0.875 | 0.866 | 0.884 | 0.926 | 0.875 | 0.978 | 0.933 | 0.864 | 1 |
| SLC1A2, IL1R2, OLAH, RETN | | | | | | | | | |
| RSAD2, OAS1, IFIT1, SIGLEC1, | 0.875 | 0.866 | 0.884 | 0.912 | 0.849 | 0.975 | 0.963 | 0.914 | 1 |
| ISG15, IL1R2, OLAH, FAM20A | | | | | | | | | |
| IFI27, OAS1, IFIT1, ISG15, HERC6, | 0.875 | 0.865 | 0.885 | 0.922 | 0.866 | 0.978 | 0.930 | 0.849 | 1 |
| SLC1A2, FAM20A, RETN | | | | | | | | | |
| IFI27, IFI44L, ISG15, HERC6, | 0.875 | 0.866 | 0.884 | 0.928 | 0.879 | 0.977 | 0.927 | 0.860 | 0.994 |
| SLC1A2, IL1R2, OLAH, MMP8 | | | | | | | | | |
| IFI27, IFIT1, IFI44L, ISG15, | 0.875 | 0.866 | 0.884 | 0.921 | 0.867 | 0.975 | 0.942 | 0.885 | 0.999 |
| HERC6, IL1R2, RETN, MMP8 | | | | | | | | | |
| RSAD2, IFI27, ISG15, SLC1A2, | 0.875 | 0.865 | 0.884 | 0.927 | 0.875 | 0.978 | 0.939 | 0.876 | 1 |
| IL1R2, OLAH, RETN, MMP8 | | | | | | | | | |
| IFI27, OAS1, IFIT1, IFI44L, | 0.875 | 0.865 | 0.884 | 0.925 | 0.874 | 0.976 | 0.924 | 0.839 | 1 |
| SLC1A2, IL1R2, FAM20A, RETN | | | | | | | | | |
| RSAD2, IFI27, IFIT1, IFI44L, | 0.875 | 0.865 | 0.884 | 0.918 | 0.859 | 0.978 | 0.949 | 0.890 | 1 |
| ISG15, OLAH, RETN, MMP8 | | | | | | | | | |
| IFI27, OAS1, IFI44L, ISG15, | 0.875 | 0.865 | 0.885 | 0.930 | 0.884 | 0.977 | 0.926 | 0.839 | 1 |
| SLC1A2, IL1R2, FAM20A, MMP8 | | | | | | | | | |
| RSAD2, IFIT1, SIGLEC1, HERC6, | 0.875 | 0.865 | 0.885 | 0.923 | 0.872 | 0.974 | 0.946 | 0.876 | 1 |
| SLC1A2, IL1R2, OLAH, FAM20A | | | | | | | | | |
| RSAD2, IFI27, IFIT1, IFI44L, | 0.875 | 0.866 | 0.883 | 0.927 | 0.877 | 0.978 | 0.946 | 0.891 | 1 |
| SIGLEC1, SLC1A2, IL1R2, OLAH | | | | | | | | | |
| RSAD2, IFI27, IFI44L, SIGLEC1, | 0.875 | 0.866 | 0.884 | 0.922 | 0.868 | 0.976 | 0.958 | 0.914 | 1 |
| ISG15, IL1R2, RETN, MMP8 | | | | | | | | | |
| RSAD2, IFI27, OAS1, IFIT1, ISG15, | 0.875 | 0.866 | 0.884 | 0.919 | 0.863 | 0.975 | 0.942 | 0.883 | 1 |
| IL1R2, OLAH, RETN | | | | | | | | | |
| RSAD2, OAS1, IFI44L, SIGLEC1, | 0.875 | 0.865 | 0.884 | 0.917 | 0.859 | 0.975 | 0.961 | 0.902 | 1 |
| ISG15, IL1R2, FAM20A, MMP8 | | | | | | | | | |
| RSAD2, IFI27, OAS1, IFIT1, | 0.875 | 0.866 | 0.884 | 0.926 | 0.876 | 0.976 | 0.938 | 0.874 | 1 |
| SLC1A2, IL1R2, OLAH, MMP8 | | | | | | | | | |
| RSAD2, IFI27, IFIT1, IFI44L, | 0.875 | 0.865 | 0.884 | 0.927 | 0.877 | 0.978 | 0.930 | 0.865 | 0.995 |
| HERC6, SLC1A2, OLAH, MMP8 | | | | | | | | | |
| IFI27, OAS1, ISG15, SLC1A2, | 0.875 | 0.865 | 0.884 | 0.926 | 0.875 | 0.978 | 0.934 | 0.868 | 0.999 |
| IL1R2, OLAH, RETN, MMP8 | | | | | | | | | |
| IFI27, IFIT1, IFI44L, ISG15, | 0.875 | 0.865 | 0.884 | 0.928 | 0.878 | 0.979 | 0.929 | 0.865 | 0.994 |
| HERC6, SLC1A2, OLAH, MMP8 | | | | | | | | | |
| RSAD2, IFI27, IFI44L, ISG15, | 0.875 | 0.865 | 0.884 | 0.928 | 0.878 | 0.978 | 0.926 | 0.859 | 0.993 |
| HERC6, SLC1A2, OLAH, MMP8 | | | | | | | | | |
| RSAD2, IFI27, OAS1, IFIT1, | 0.875 | 0.866 | 0.884 | 0.917 | 0.860 | 0.974 | 0.946 | 0.886 | 1 |
| IFI44L, IL1R2, OLAH, RETN | | | | | | | | | |
| RSAD2, IFIT1, SIGLEC1, ISG15, | 0.875 | 0.865 | 0.884 | 0.922 | 0.869 | 0.975 | 0.949 | 0.887 | 1 |
| SLC1A2, OLAH, FAM20A, MMP8 | | | | | | | | | |
| OAS1, IFI44L, SIGLEC1, SLC1A2, | 0.875 | 0.865 | 0.884 | 0.924 | 0.876 | 0.973 | 0.958 | 0.897 | 1 |
| IL1R2, OLAH, FAM20A, MMP8 | | | | | | | | | |
| RSAD2, OAS1, IFIT1, SIGLEC1, | 0.875 | 0.865 | 0.885 | 0.912 | 0.848 | 0.976 | 0.959 | 0.910 | 1 |
| ISG15, OLAH, FAM20A, MMP8 | | | | | | | | | |
| SIGLEC1, ISG15, SLC1A2, IL1R2, | 0.875 | 0.865 | 0.884 | 0.923 | 0.874 | 0.973 | 0.957 | 0.890 | 1 |
| OLAH, FAM20A, RETN, MMP8 | | | | | | | | | |
| RSAD2, SIGLEC1, HERC6, | 0.875 | 0.865 | 0.885 | 0.925 | 0.876 | 0.974 | 0.957 | 0.890 | 1 |
| SLC1A2, IL1R2, FAM20A, RETN, MMP8 | | | | | | | | | |
| RSAD2, IFI27, OAS1, IFIT1, | 0.875 | 0.865 | 0.884 | 0.923 | 0.868 | 0.978 | 0.941 | 0.877 | 1 |
| SLC1A2, OLAH, RETN, MMP8 | | | | | | | | | |
| RSAD2, IFI27, OAS1, IFIT1, | 0.875 | 0.865 | 0.884 | 0.916 | 0.858 | 0.974 | 0.935 | 0.875 | 0.994 |
| IFI44L, ISG15, OLAH, MMP8 | | | | | | | | | |
| IFI27, IFIT1, IFI44L, SIGLEC1, | 0.875 | 0.865 | 0.884 | 0.927 | 0.877 | 0.977 | 0.946 | 0.893 | 0.998 |
| HERC6, SLC1A2, IL1R2, OLAH | | | | | | | | | |
| RSAD2, IFI27, OAS1, IFIT1, | 0.875 | 0.866 | 0.883 | 0.919 | 0.868 | 0.969 | 0.951 | 0.905 | 0.997 |
| IFI44L, SIGLEC1, IL1R2, MMP8 | | | | | | | | | |
| RSAD2, IFI27, OAS1, SIGLEC1, | 0.875 | 0.865 | 0.885 | 0.923 | 0.866 | 0.979 | 0.943 | 0.869 | 1 |
| ISG15, SLC1A2, FAM20A, RETN | | | | | | | | | |
| RSAD2, IFI27, OAS1, IFIT1, | 0.875 | 0.866 | 0.883 | 0.918 | 0.864 | 0.973 | 0.938 | 0.881 | 0.995 |
| IFI44L, ISG15, IL1R2, OLAH | | | | | | | | | |
| RSAD2, OAS1, IFIT1, SIGLEC1, | 0.875 | 0.865 | 0.884 | 0.914 | 0.854 | 0.975 | 0.946 | 0.886 | 1 |
| HERC6, OLAH, FAM20A, RETN | | | | | | | | | |
| RSAD2, IFI27, OAS1, IFI44L, | 0.875 | 0.865 | 0.884 | 0.918 | 0.859 | 0.978 | 0.950 | 0.894 | 1 |
| ISG15, OLAH, RETN, MMP8 | | | | | | | | | |
| IFI27, IFI44L, SIGLEC1, ISG15, | 0.875 | 0.866 | 0.883 | 0.928 | 0.880 | 0.976 | 0.943 | 0.890 | 0.997 |
| HERC6, SLC1A2, IL1R2, RETN | | | | | | | | | |
| RSAD2, SIGLEC1, ISG15, IL1R2, | 0.875 | 0.865 | 0.884 | 0.918 | 0.860 | 0.976 | 0.965 | 0.913 | 1 |
| OLAH, FAM20A, RETN, MMP8 | | | | | | | | | |
| RSAD2, IFI27, IFIT1, ISG15, | 0.875 | 0.866 | 0.883 | 0.920 | 0.866 | 0.974 | 0.948 | 0.896 | 1.000 |
| HERC6, IL1R2, RETN, MMP8 | | | | | | | | | |
| IFI27, IFIT1, IFI44L, ISG15, | 0.875 | 0.865 | 0.884 | 0.926 | 0.876 | 0.976 | 0.924 | 0.837 | 1 |
| SLC1A2, IL1R2, FAM20A, RETN | | | | | | | | | |
| IFI27, IFIT1, IFI44L, SIGLEC1, | 0.874 | 0.866 | 0.883 | 0.927 | 0.878 | 0.976 | 0.946 | 0.895 | 0.997 |
| HERC6, SLC1A2, IL1R2, RETN | | | | | | | | | |
| RSAD2, IFI27, IFIT1, ISG15, | 0.874 | 0.866 | 0.883 | 0.928 | 0.878 | 0.979 | 0.935 | 0.869 | 1 |
| SLC1A2, IL1R2, OLAH, RETN | | | | | | | | | |
| RSAD2, IFI27, IFIT1, IFI44L, | 0.874 | 0.866 | 0.883 | 0.920 | 0.865 | 0.974 | 0.947 | 0.896 | 0.998 |
| SIGLEC1, ISG15, IL1R2, RETN | | | | | | | | | |
| OAS1, IFIT1, SIGLEC1, SLC1A2, | 0.874 | 0.865 | 0.884 | 0.928 | 0.881 | 0.975 | 0.959 | 0.893 | 1 |
| IL1R2, FAM20A, RETN, MMP8 | | | | | | | | | |
| IFIT1, IFI44L, SIGLEC1, HERC6, | 0.874 | 0.865 | 0.884 | 0.923 | 0.870 | 0.976 | 0.945 | 0.873 | 1 |
| SLC1A2, FAM20A, RETN, MMP8 | | | | | | | | | |
| RSAD2, IFI27, OAS1, IFI44L, | 0.874 | 0.865 | 0.884 | 0.925 | 0.873 | 0.978 | 0.929 | 0.861 | 0.998 |
| ISG15, SLC1A2, OLAH, MMP8 | | | | | | | | | |
| RSAD2, OAS1, IFIT1, SIGLEC1, | 0.874 | 0.865 | 0.884 | 0.922 | 0.871 | 0.974 | 0.942 | 0.876 | 1 |
| HERC6, SLC1A2, OLAH, FAM20A | | | | | | | | | |
| RSAD2, IFI27, OAS1, IFIT1, ISG15, | 0.874 | 0.865 | 0.883 | 0.929 | 0.880 | 0.978 | 0.930 | 0.863 | 0.998 |
| SLC1A2, IL1R2, OLAH | | | | | | | | | |
| RSAD2, IFI27, OAS1, IFIT1, | 0.874 | 0.865 | 0.884 | 0.928 | 0.878 | 0.978 | 0.936 | 0.871 | 1 |
| IFI44L, HERC6, SLC1A2, OLAH | | | | | | | | | |
| RSAD2, OAS1, IFIT1, IFI44L, | 0.874 | 0.865 | 0.884 | 0.916 | 0.856 | 0.976 | 0.937 | 0.872 | 1 |
| SIGLEC1, HERC6, FAM20A, RETN | | | | | | | | | |
| RSAD2, IFI27, IFIT1, IFI44L, | 0.874 | 0.865 | 0.884 | 0.922 | 0.865 | 0.978 | 0.939 | 0.864 | 1 |
| SIGLEC1, ISG15, SLC1A2, FAM20A | | | | | | | | | |
| RSAD2, IFI27, OAS1, ISG15, | 0.874 | 0.865 | 0.883 | 0.928 | 0.876 | 0.979 | 0.930 | 0.858 | 1 |
| SLC1A2, IL1R2, OLAH, RETN | | | | | | | | | |
| RSAD2, IFI27, IFIT1, ISG15, | 0.874 | 0.865 | 0.884 | 0.926 | 0.872 | 0.980 | 0.940 | 0.878 | 1 |
| SLC1A2, OLAH, RETN, MMP8 | | | | | | | | | |
| RSAD2, IFI27, OAS1, IFI44L, | 0.874 | 0.864 | 0.884 | 0.929 | 0.881 | 0.977 | 0.934 | 0.868 | 1.000 |
| HERC6, SLC1A2, IL1R2, OLAH | | | | | | | | | |
| RSAD2, IFI27, IFI44L, ISG15, | 0.874 | 0.865 | 0.884 | 0.927 | 0.875 | 0.980 | 0.938 | 0.874 | 1 |
| SLC1A2, OLAH, RETN, MMP8 | | | | | | | | | |
| OAS1, SIGLEC1, HERC6, SLC1A2, | 0.874 | 0.864 | 0.884 | 0.927 | 0.879 | 0.975 | 0.959 | 0.895 | 1 |
| IL1R2, FAM20A, RETN, MMP8 | | | | | | | | | |
| RSAD2, IFI27, IFI44L, ISG15, | 0.874 | 0.865 | 0.883 | 0.929 | 0.879 | 0.978 | 0.930 | 0.865 | 0.996 |
| SLC1A2, IL1R2, OLAH, MMP8 | | | | | | | | | |
| OAS1, IFIT1, SIGLEC1, ISG15, | 0.874 | 0.865 | 0.884 | 0.924 | 0.876 | 0.973 | 0.945 | 0.873 | 1 |
| HERC6, SLC1A2, IL1R2, FAM20A | | | | | | | | | |
| IFI27, IFIT1, IFI44L, ISG15, | 0.874 | 0.865 | 0.884 | 0.925 | 0.870 | 0.979 | 0.942 | 0.881 | 1 |
| SLC1A2, OLAH, RETN, MMP8 | | | | | | | | | |
| IFI27, OAS1, IFIT1, IFI44L, ISG15, | 0.874 | 0.865 | 0.883 | 0.917 | 0.861 | 0.974 | 0.937 | 0.875 | 0.999 |
| IL1R2, OLAH, RETN | | | | | | | | | |
| RSAD2, IFI27, OAS1, SLC1A2, | 0.874 | 0.865 | 0.883 | 0.929 | 0.880 | 0.978 | 0.927 | 0.842 | 1 |
| IL1R2, FAM20A, RETN, MMP8 | | | | | | | | | |
| RSAD2, IFI27, IFI44L, SLC1A2, | 0.874 | 0.865 | 0.884 | 0.925 | 0.873 | 0.978 | 0.941 | 0.878 | 1 |
| IL1R2, OLAH, RETN, MMP8 | | | | | | | | | |
| IFI27, IFIT1, IFI44L, SIGLEC1, | 0.874 | 0.866 | 0.883 | 0.925 | 0.876 | 0.974 | 0.945 | 0.889 | 1 |
| ISG15, SLC1A2, IL1R2, RETN | | | | | | | | | |
| RSAD2, IFI27, IFIT1, IFI44L, | 0.874 | 0.865 | 0.884 | 0.923 | 0.867 | 0.979 | 0.945 | 0.884 | 1 |
| SLC1A2, OLAH, RETN, MMP8 | | | | | | | | | |
| RSAD2, IFI27, OAS1, IFIT1, | 0.874 | 0.865 | 0.884 | 0.915 | 0.853 | 0.977 | 0.933 | 0.860 | 1 |
| IFI44L, HERC6, RETN, MMP8 | | | | | | | | | |
| RSAD2, IFI27, OAS1, IFIT1, | 0.874 | 0.866 | 0.883 | 0.919 | 0.870 | 0.969 | 0.938 | 0.882 | 0.994 |
| IFI44L, HERC6, IL1R2, MMP8 | | | | | | | | | |
| IFI27, OAS1, IFIT1, IFI44L, | 0.874 | 0.864 | 0.884 | 0.922 | 0.866 | 0.977 | 0.941 | 0.877 | 1 |
| SLC1A2, OLAH, RETN, MMP8 | | | | | | | | | |
| RSAD2, IFIT1, SIGLEC1, ISG15, | 0.874 | 0.865 | 0.884 | 0.926 | 0.877 | 0.974 | 0.946 | 0.874 | 1 |
| HERC6, SLC1A2, IL1R2, FAM20A | | | | | | | | | |
| IFI27, IFI44L, SIGLEC1, ISG15, | 0.874 | 0.866 | 0.883 | 0.929 | 0.882 | 0.975 | 0.961 | 0.918 | 1 |
| SLC1A2, IL1R2, RETN, MMP8 | | | | | | | | | |
| RSAD2, IFIT1, IFI44L, SIGLEC1, | 0.874 | 0.865 | 0.883 | 0.918 | 0.859 | 0.976 | 0.940 | 0.876 | 1 |
| ISG15, HERC6, FAM20A, RETN | | | | | | | | | |
| IFI27, OAS1, IFIT1, IFI44L, | 0.874 | 0.865 | 0.883 | 0.923 | 0.873 | 0.973 | 0.950 | 0.895 | 1 |
| SIGLEC1, SLC1A2, IL1R2, RETN | | | | | | | | | |
| IFI27, OAS1, IFIT1, IFI44L, ISG15, | 0.874 | 0.865 | 0.884 | 0.927 | 0.875 | 0.979 | 0.929 | 0.862 | 0.997 |
| SLC1A2, OLAH, MMP8 | | | | | | | | | |
| RSAD2, IFI27, IFIT1, SIGLEC1, | 0.874 | 0.865 | 0.883 | 0.927 | 0.881 | 0.974 | 0.951 | 0.903 | 0.999 |
| HERC6, SLC1A2, IL1R2, RETN | | | | | | | | | |
| RSAD2, IFI27, OAS1, IFIT1, | 0.874 | 0.865 | 0.883 | 0.927 | 0.875 | 0.979 | 0.941 | 0.879 | 1 |
| IFI44L, SLC1A2, OLAH, MMP8 | | | | | | | | | |
| RSAD2, IFI27, OAS1, IFIT1, | 0.874 | 0.865 | 0.883 | 0.926 | 0.874 | 0.978 | 0.938 | 0.869 | 1 |
| SLC1A2, IL1R2, OLAH, RETN | | | | | | | | | |
| RSAD2, IFI27, IFIT1, IFI44L, | 0.874 | 0.865 | 0.883 | 0.916 | 0.859 | 0.974 | 0.938 | 0.877 | 1.000 |
| ISG15, IL1R2, OLAH, RETN | | | | | | | | | |
| RSAD2, IFI27, IFIT1, IFI44L, | 0.874 | 0.865 | 0.884 | 0.923 | 0.866 | 0.979 | 0.940 | 0.866 | 1 |
| SIGLEC1, SLC1A2, FAM20A, MMP8 | | | | | | | | | |
| IFI27, IFIT1, IFI44L, SLC1A2, | 0.874 | 0.864 | 0.884 | 0.924 | 0.871 | 0.977 | 0.946 | 0.888 | 1 |
| IL1R2, OLAH, RETN, MMP8 | | | | | | | | | |
| IFI27, IFIT1, IFI44L, HERC6, | 0.874 | 0.865 | 0.883 | 0.924 | 0.871 | 0.976 | 0.935 | 0.869 | 1 |
| SLC1A2, IL1R2, OLAH, RETN | | | | | | | | | |
| IFI27, OAS1, IFI44L, ISG15, | 0.874 | 0.864 | 0.884 | 0.923 | 0.868 | 0.979 | 0.935 | 0.854 | 1 |
| HERC6, SLC1A2, FAM20A, RETN | | | | | | | | | |
| IFI27, IFI44L, SIGLEC1, ISG15, | 0.874 | 0.863 | 0.885 | 0.924 | 0.868 | 0.980 | 0.948 | 0.872 | 1 |
| SLC1A2, FAM20A, RETN, MMP8 | | | | | | | | | |
| IFI44L, SIGLEC1, SLC1A2, IL1R2, | 0.874 | 0.864 | 0.883 | 0.923 | 0.872 | 0.973 | 0.957 | 0.891 | 1 |
| OLAH, FAM20A, RETN, MMP8 | | | | | | | | | |
| RSAD2, IFIT1, IFI44L, SIGLEC1, | 0.874 | 0.865 | 0.883 | 0.916 | 0.856 | 0.977 | 0.943 | 0.884 | 1 |
| HERC6, OLAH, FAM20A, RETN | | | | | | | | | |
| IFI27, OAS1, ISG15, SLC1A2, | 0.874 | 0.865 | 0.883 | 0.928 | 0.878 | 0.978 | 0.927 | 0.841 | 1 |
| IL1R2, FAM20A, RETN, MMP8 | | | | | | | | | |
| RSAD2, IFIT1, SIGLEC1, ISG15, | 0.874 | 0.865 | 0.883 | 0.916 | 0.856 | 0.976 | 0.948 | 0.889 | 1 |
| HERC6, OLAH, FAM20A, RETN | | | | | | | | | |
| RSAD2, IFIT1, IFI44L, SIGLEC1, | 0.874 | 0.865 | 0.883 | 0.911 | 0.847 | 0.975 | 0.962 | 0.913 | 1 |
| ISG15, IL1R2, OLAH, FAM20A | | | | | | | | | |
| OAS1, IFIT1, SIGLEC1, ISG15, | 0.874 | 0.864 | 0.884 | 0.922 | 0.870 | 0.973 | 0.937 | 0.867 | 1 |
| HERC6, SLC1A2, OLAH, FAM20A | | | | | | | | | |
| OAS1, IFIT1, SIGLEC1, ISG15, | 0.874 | 0.865 | 0.883 | 0.911 | 0.849 | 0.974 | 0.961 | 0.910 | 1 |
| IL1R2, OLAH, FAM20A, RETN | | | | | | | | | |
| OAS1, IFIT1, IFI44L, SIGLEC1, | 0.874 | 0.865 | 0.883 | 0.910 | 0.847 | 0.974 | 0.960 | 0.907 | 1 |
| IL1R2, OLAH, FAM20A, RETN | | | | | | | | | |
| IFI27, OAS1, IFI44L, ISG15, | 0.874 | 0.864 | 0.884 | 0.927 | 0.878 | 0.976 | 0.928 | 0.861 | 0.995 |
| HERC6, SLC1A2, IL1R2, OLAH | | | | | | | | | |
| RSAD2, IFI27, IFI44L, SIGLEC1, | 0.874 | 0.865 | 0.882 | 0.927 | 0.878 | 0.976 | 0.945 | 0.892 | 0.997 |
| HERC6, SLC1A2, IL1R2, RETN | | | | | | | | | |
| RSAD2, IFIT1, SIGLEC1, ISG15, | 0.874 | 0.864 | 0.884 | 0.922 | 0.869 | 0.975 | 0.936 | 0.862 | 1 |
| HERC6, SLC1A2, OLAH, FAM20A | | | | | | | | | |
| IFI27, OAS1, IFIT1, HERC6, | 0.874 | 0.865 | 0.883 | 0.923 | 0.873 | 0.974 | 0.938 | 0.873 | 1 |
| SLC1A2, IL1R2, RETN, MMP8 | | | | | | | | | |
| IFI27, OAS1, IFIT1, ISG15, | 0.874 | 0.864 | 0.883 | 0.927 | 0.878 | 0.977 | 0.925 | 0.841 | 1 |
| SLC1A2, IL1R2, FAM20A, RETN | | | | | | | | | |
| OAS1, IFIT1, SIGLEC1, HERC6, | 0.874 | 0.864 | 0.883 | 0.920 | 0.866 | 0.974 | 0.942 | 0.870 | 1 |
| SLC1A2, FAM20A, RETN, MMP8 | | | | | | | | | |
| OAS1, IFIT1, IFI44L, SIGLEC1, | 0.874 | 0.864 | 0.884 | 0.915 | 0.852 | 0.979 | 0.962 | 0.916 | 1 |
| OLAH, FAM20A, RETN, MMP8 | | | | | | | | | |
| RSAD2, IFIT1, IFI44L, SIGLEC1, | 0.874 | 0.865 | 0.883 | 0.919 | 0.860 | 0.979 | 0.942 | 0.874 | 1 |
| HERC6, FAM20A, RETN, MMP8 | | | | | | | | | |
| RSAD2, IFIT1, IFI44L, SIGLEC1, | 0.874 | 0.865 | 0.883 | 0.919 | 0.861 | 0.976 | 0.952 | 0.895 | 1 |
| HERC6, IL1R2, FAM20A, RETN | | | | | | | | | |
| RSAD2, IFIT1, SIGLEC1, SLC1A2, | 0.874 | 0.865 | 0.883 | 0.927 | 0.879 | 0.975 | 0.959 | 0.893 | 1 |
| IL1R2, FAM20A, RETN, MMP8 | | | | | | | | | |
| IFI27, OAS1, IFIT1, IFI44L, ISG15, | 0.874 | 0.864 | 0.883 | 0.927 | 0.878 | 0.975 | 0.926 | 0.842 | 1 |
| SLC1A2, IL1R2, FAM20A | | | | | | | | | |
| RSAD2, IFI27, IFI44L, ISG15, | 0.874 | 0.864 | 0.883 | 0.929 | 0.880 | 0.978 | 0.925 | 0.856 | 0.994 |
| HERC6, SLC1A2, IL1R2, OLAH | | | | | | | | | |
| RSAD2, IFI27, OAS1, SIGLEC1, | 0.874 | 0.864 | 0.883 | 0.922 | 0.868 | 0.976 | 0.965 | 0.920 | 1 |
| ISG15, IL1R2, RETN, MMP8 | | | | | | | | | |
| IFI27, OAS1, IFIT1, IFI44L, | 0.874 | 0.865 | 0.882 | 0.922 | 0.874 | 0.970 | 0.945 | 0.895 | 0.995 |
| SIGLEC1, ISG15, IL1R2, MMP8 | | | | | | | | | |
| RSAD2, IFI27, OAS1, IFIT1, | 0.874 | 0.865 | 0.882 | 0.918 | 0.867 | 0.970 | 0.948 | 0.900 | 0.996 |
| IFI44L, SIGLEC1, ISG15, IL1R2 | | | | | | | | | |
| IFI27, OAS1, IFIT1, SIGLEC1, | 0.874 | 0.865 | 0.883 | 0.926 | 0.882 | 0.971 | 0.949 | 0.899 | 0.999 |
| HERC6, SLC1A2, IL1R2, MMP8 | | | | | | | | | |
| IFI27, IFIT1, IFI44L, ISG15, | 0.874 | 0.865 | 0.883 | 0.927 | 0.876 | 0.978 | 0.935 | 0.870 | 1 |
| SLC1A2, IL1R2, OLAH, RETN | | | | | | | | | |
| RSAD2, IFI27, IFI44L, SIGLEC1, | 0.874 | 0.864 | 0.883 | 0.917 | 0.859 | 0.975 | 0.945 | 0.890 | 0.999 |
| ISG15, HERC6, RETN, MMP8 | | | | | | | | | |
| IFIT1, SIGLEC1, ISG15, SLC1A2, | 0.874 | 0.864 | 0.883 | 0.927 | 0.881 | 0.974 | 0.958 | 0.892 | 1 |
| IL1R2, FAM20A, RETN, MMP8 | | | | | | | | | |
| RSAD2, IFIT1, IFI44L, SIGLEC1, | 0.874 | 0.864 | 0.883 | 0.926 | 0.877 | 0.974 | 0.941 | 0.869 | 1 |
| HERC6, SLC1A2, IL1R2, FAM20A | | | | | | | | | |
| RSAD2, IFI27, IFIT1, IFI44L, | 0.874 | 0.865 | 0.883 | 0.926 | 0.874 | 0.978 | 0.929 | 0.863 | 0.996 |
| ISG15, SLC1A2, OLAH, MMP8 | | | | | | | | | |
| IFI27, IFIT1, IFI44L, SIGLEC1, | 0.874 | 0.865 | 0.882 | 0.926 | 0.878 | 0.975 | 0.958 | 0.912 | 1 |
| SLC1A2, IL1R2, RETN, MMP8 | | | | | | | | | |
| OAS1, IFI44L, SIGLEC1, ISG15, | 0.874 | 0.864 | 0.883 | 0.916 | 0.857 | 0.975 | 0.961 | 0.902 | 1 |
| IL1R2, FAM20A, RETN, MMP8 | | | | | | | | | |
| RSAD2, SIGLEC1, SLC1A2, IL1R2, | 0.874 | 0.864 | 0.883 | 0.924 | 0.875 | 0.973 | 0.961 | 0.903 | 1 |
| OLAH, FAM20A, RETN, MMP8 | | | | | | | | | |
| RSAD2, IFI27, IFIT1, IFI44L, | 0.874 | 0.864 | 0.883 | 0.928 | 0.877 | 0.978 | 0.929 | 0.865 | 0.994 |
| ISG15, HERC6, SLC1A2, OLAH | | | | | | | | | |
| SIGLEC1, ISG15, HERC6, SLC1A2, | 0.873 | 0.864 | 0.883 | 0.925 | 0.876 | 0.974 | 0.955 | 0.885 | 1 |
| IL1R2, FAM20A, RETN, MMP8 | | | | | | | | | |
| IFIT1, IFI44L, SIGLEC1, ISG15, | 0.873 | 0.864 | 0.883 | 0.914 | 0.851 | 0.977 | 0.957 | 0.907 | 1 |
| OLAH, FAM20A, RETN, MMP8 | | | | | | | | | |
| RSAD2, IFI27, OAS1, ISG15, | 0.873 | 0.864 | 0.883 | 0.927 | 0.873 | 0.980 | 0.938 | 0.872 | 1 |
| SLC1A2, OLAH, RETN, MMP8 | | | | | | | | | |
| IFI27, OAS1, IFI44L, ISG15, | 0.873 | 0.864 | 0.883 | 0.927 | 0.877 | 0.977 | 0.924 | 0.839 | 1 |
| SLC1A2, IL1R2, FAM20A, RETN | | | | | | | | | |
| IFI27, OAS1, IFIT1, SIGLEC1, | 0.873 | 0.865 | 0.882 | 0.925 | 0.877 | 0.974 | 0.953 | 0.898 | 1 |
| ISG15, SLC1A2, IL1R2, RETN | | | | | | | | | |
| RSAD2, IFI27, OAS1, IFIT1, | 0.873 | 0.864 | 0.883 | 0.926 | 0.878 | 0.975 | 0.926 | 0.842 | 1 |
| IFI44L, SLC1A2, IL1R2, FAM20A | | | | | | | | | |
| RSAD2, IFI27, IFIT1, SIGLEC1, | 0.873 | 0.865 | 0.882 | 0.922 | 0.875 | 0.969 | 0.946 | 0.897 | 0.995 |
| ISG15, HERC6, IL1R2, MMP8 | | | | | | | | | |
| RSAD2, IFI27, IFIT1, IFI44L, | 0.873 | 0.864 | 0.883 | 0.926 | 0.875 | 0.977 | 0.925 | 0.840 | 1 |
| SLC1A2, IL1R2, FAM20A, RETN | | | | | | | | | |
| RSAD2, IFI27, IFIT1, IFI44L, | 0.873 | 0.864 | 0.882 | 0.926 | 0.876 | 0.976 | 0.936 | 0.875 | 0.996 |
| SLC1A2, IL1R2, OLAH, MMP8 | | | | | | | | | |
| RSAD2, IFI27, IFIT1, SIGLEC1, | 0.873 | 0.864 | 0.882 | 0.922 | 0.868 | 0.975 | 0.963 | 0.922 | 1 |
| ISG15, IL1R2, RETN, MMP8 | | | | | | | | | |
| IFI27, IFIT1, IFI44L, ISG15, | 0.873 | 0.863 | 0.883 | 0.921 | 0.864 | 0.978 | 0.935 | 0.853 | 1 |
| HERC6, SLC1A2, FAM20A, RETN | | | | | | | | | |
| IFI27, OAS1, IFIT1, ISG15, HERC6, | 0.873 | 0.864 | 0.882 | 0.924 | 0.874 | 0.974 | 0.939 | 0.876 | 1 |
| SLC1A2, IL1R2, RETN | | | | | | | | | |
| IFIT1, IFI44L, SIGLEC1, ISG15, | 0.873 | 0.864 | 0.882 | 0.908 | 0.844 | 0.971 | 0.958 | 0.906 | 1 |
| IL1R2, OLAH, FAM20A, RETN | | | | | | | | | |
| IFI27, OAS1, IFI44L, ISG15, | 0.873 | 0.864 | 0.883 | 0.926 | 0.873 | 0.979 | 0.939 | 0.875 | 1 |
| SLC1A2, OLAH, RETN, MMP8 | | | | | | | | | |
| OAS1, SIGLEC1, ISG15, SLC1A2, | 0.873 | 0.864 | 0.883 | 0.925 | 0.877 | 0.973 | 0.958 | 0.893 | 1 |
| IL1R2, FAM20A, RETN, MMP8 | | | | | | | | | |
| RSAD2, IFI27, IFI44L, ISG15, | 0.873 | 0.864 | 0.882 | 0.927 | 0.876 | 0.978 | 0.932 | 0.863 | 1.000 |
| SLC1A2, IL1R2, OLAH, RETN | | | | | | | | | |
| RSAD2, OAS1, IFI44L, SIGLEC1, | 0.873 | 0.864 | 0.883 | 0.923 | 0.874 | 0.971 | 0.961 | 0.899 | 1 |
| SLC1A2, IL1R2, FAM20A, MMP8 | | | | | | | | | |
| RSAD2, IFI27, OAS1, IFIT1, | 0.873 | 0.865 | 0.882 | 0.918 | 0.864 | 0.973 | 0.954 | 0.902 | 1 |
| SIGLEC1, ISG15, IL1R2, RETN | | | | | | | | | |
| IFI27, OAS1, IFIT1, SIGLEC1, | 0.873 | 0.863 | 0.883 | 0.921 | 0.865 | 0.977 | 0.941 | 0.869 | 1 |
| ISG15, SLC1A2, FAM20A, MMP8 | | | | | | | | | |
| RSAD2, OAS1, SIGLEC1, ISG15, | 0.873 | 0.863 | 0.883 | 0.924 | 0.876 | 0.972 | 0.959 | 0.893 | 1 |
| SLC1A2, IL1R2, FAM20A, MMP8 | | | | | | | | | |
| RSAD2, OAS1, IFI44L, SIGLEC1, | 0.873 | 0.863 | 0.883 | 0.919 | 0.862 | 0.976 | 0.962 | 0.906 | 1 |
| IL1R2, FAM20A, RETN, MMP8 | | | | | | | | | |
| RSAD2, IFI27, IFI44L, SIGLEC1, | 0.873 | 0.863 | 0.883 | 0.922 | 0.866 | 0.978 | 0.942 | 0.867 | 1 |
| ISG15, SLC1A2, FAM20A, RETN | | | | | | | | | |
| IFI27, OAS1, IFIT1, IFI44L, | 0.873 | 0.863 | 0.883 | 0.926 | 0.877 | 0.975 | 0.935 | 0.870 | 1 |
| HERC6, SLC1A2, IL1R2, OLAH | | | | | | | | | |
| RSAD2, IFI27, OAS1, IFI44L, | 0.873 | 0.864 | 0.883 | 0.927 | 0.879 | 0.975 | 0.928 | 0.844 | 1 |
| ISG15, SLC1A2, IL1R2, FAM20A | | | | | | | | | |
| RSAD2, IFI27, OAS1, IFIT1, | 0.873 | 0.864 | 0.882 | 0.915 | 0.856 | 0.975 | 0.940 | 0.876 | 1 |
| IFI44L, ISG15, OLAH, RETN | | | | | | | | | |
| IFIT1, IFI44L, SIGLEC1, ISG15, | 0.873 | 0.864 | 0.882 | 0.919 | 0.863 | 0.975 | 0.935 | 0.871 | 0.999 |
| SLC1A2, OLAH, FAM20A, RETN | | | | | | | | | |
| RSAD2, OAS1, SIGLEC1, HERC6, | 0.873 | 0.863 | 0.883 | 0.920 | 0.868 | 0.972 | 0.945 | 0.878 | 1 |
| SLC1A2, OLAH, FAM20A, MMP8 | | | | | | | | | |
| RSAD2, OAS1, IFIT1, SIGLEC1, | 0.873 | 0.864 | 0.883 | 0.926 | 0.877 | 0.974 | 0.949 | 0.884 | 1 |
| HERC6, SLC1A2, IL1R2, FAM20A | | | | | | | | | |
| OAS1, IFIT1, SIGLEC1, ISG15, | 0.873 | 0.863 | 0.883 | 0.912 | 0.848 | 0.977 | 0.961 | 0.912 | 1 |
| OLAH, FAM20A, RETN, MMP8 | | | | | | | | | |
| RSAD2, IFI27, IFIT1, IFI44L, | 0.873 | 0.864 | 0.883 | 0.925 | 0.876 | 0.975 | 0.927 | 0.842 | 1 |
| ISG15, SLC1A2, IL1R2, FAM20A | | | | | | | | | |
| OAS1, SIGLEC1, SLC1A2, IL1R2, | 0.873 | 0.863 | 0.883 | 0.924 | 0.874 | 0.973 | 0.960 | 0.902 | 1 |
| OLAH, FAM20A, RETN, MMP8 | | | | | | | | | |
| IFI27, IFIT1, IFI44L, HERC6, | 0.873 | 0.864 | 0.882 | 0.924 | 0.874 | 0.975 | 0.933 | 0.870 | 0.995 |
| SLC1A2, IL1R2, OLAH, MMP8 | | | | | | | | | |
| RSAD2, IFI27, OAS1, IFI44L, | 0.873 | 0.863 | 0.883 | 0.923 | 0.867 | 0.980 | 0.939 | 0.862 | 1 |
| SIGLEC1, SLC1A2, FAM20A, RETN | | | | | | | | | |
| IFI27, OAS1, IFIT1, IFI44L, | 0.873 | 0.864 | 0.882 | 0.926 | 0.876 | 0.976 | 0.937 | 0.872 | 1 |
| SLC1A2, IL1R2, OLAH, MMP8 | | | | | | | | | |
| RSAD2, IFI27, OAS1, ISG15, | 0.873 | 0.863 | 0.883 | 0.923 | 0.867 | 0.979 | 0.932 | 0.850 | 1 |
| HERC6, SLC1A2, FAM20A, RETN | | | | | | | | | |
| OAS1, IFI44L, SIGLEC1, IL1R2, | 0.873 | 0.863 | 0.883 | 0.919 | 0.861 | 0.976 | 0.964 | 0.911 | 1 |
| OLAH, FAM20A, RETN, MMP8 | | | | | | | | | |
| IFIT1, SIGLEC1, ISG15, HERC6, | 0.873 | 0.863 | 0.883 | 0.924 | 0.875 | 0.973 | 0.946 | 0.874 | 1 |
| SLC1A2, IL1R2, FAM20A, RETN | | | | | | | | | |
| RSAD2, IFI27, OAS1, IFI44L, | 0.873 | 0.864 | 0.882 | 0.930 | 0.880 | 0.979 | 0.932 | 0.864 | 0.999 |
| ISG15, SLC1A2, IL1R2, OLAH | | | | | | | | | |
| IFIT1, IFI44L, SIGLEC1, ISG15, | 0.873 | 0.864 | 0.882 | 0.920 | 0.866 | 0.973 | 0.946 | 0.886 | 1 |
| SLC1A2, IL1R2, OLAH, FAM20A | | | | | | | | | |
| RSAD2, IFI27, OAS1, IFIT1, | 0.873 | 0.864 | 0.882 | 0.925 | 0.875 | 0.975 | 0.934 | 0.862 | 1 |
| HERC6, SLC1A2, IL1R2, RETN | | | | | | | | | |
| IFI27, OAS1, IFI44L, SIGLEC1, | 0.873 | 0.864 | 0.882 | 0.928 | 0.882 | 0.974 | 0.959 | 0.910 | 1 |
| SLC1A2, IL1R2, RETN, MMP8 | | | | | | | | | |
| RSAD2, SIGLEC1, ISG15, SLC1A2, | 0.873 | 0.863 | 0.883 | 0.927 | 0.879 | 0.974 | 0.959 | 0.895 | 1 |
| IL1R2, FAM20A, RETN, MMP8 | | | | | | | | | |
| RSAD2, IFI27, IFIT1, IFI44L, | 0.873 | 0.863 | 0.882 | 0.925 | 0.875 | 0.975 | 0.932 | 0.868 | 0.995 |
| HERC6, SLC1A2, IL1R2, OLAH | | | | | | | | | |
| IFI27, SIGLEC1, ISG15, HERC6, | 0.873 | 0.862 | 0.884 | 0.924 | 0.867 | 0.982 | 0.950 | 0.874 | 1 |
| SLC1A2, FAM20A, RETN, MMP8 | | | | | | | | | |
| RSAD2, OAS1, IFIT1, IFI44L, | 0.873 | 0.864 | 0.882 | 0.912 | 0.849 | 0.975 | 0.963 | 0.914 | 1 |
| SIGLEC1, ISG15, IL1R2, FAM20A | | | | | | | | | |
| IFI27, OAS1, IFIT1, IFI44L, ISG15, | 0.873 | 0.864 | 0.882 | 0.925 | 0.873 | 0.978 | 0.936 | 0.868 | 1 |
| SLC1A2, OLAH, RETN | | | | | | | | | |
| IFI27, IFIT1, IFI44L, ISG15, | 0.873 | 0.864 | 0.882 | 0.927 | 0.878 | 0.977 | 0.929 | 0.864 | 0.995 |
| HERC6, SLC1A2, IL1R2, OLAH | | | | | | | | | |
| IFI44L, SIGLEC1, HERC6, | 0.873 | 0.863 | 0.883 | 0.923 | 0.873 | 0.973 | 0.959 | 0.893 | 1 |
| SLC1A2, IL1R2, FAM20A, RETN, MMP8 | | | | | | | | | |
| IFI27, IFIT1, SIGLEC1, ISG15, | 0.873 | 0.864 | 0.882 | 0.927 | 0.880 | 0.974 | 0.954 | 0.908 | 1 |
| HERC6, SLC1A2, IL1R2, RETN | | | | | | | | | |
| RSAD2, IFI27, OAS1, IFIT1, | 0.873 | 0.864 | 0.881 | 0.923 | 0.873 | 0.973 | 0.957 | 0.900 | 1 |
| SIGLEC1, SLC1A2, IL1R2, RETN | | | | | | | | | |
| RSAD2, IFI27, OAS1, IFI44L, | 0.873 | 0.864 | 0.882 | 0.924 | 0.873 | 0.974 | 0.932 | 0.858 | 1 |
| HERC6, SLC1A2, IL1R2, RETN | | | | | | | | | |
| IFI27, OAS1, IFI44L, SLC1A2, | 0.873 | 0.863 | 0.883 | 0.927 | 0.876 | 0.979 | 0.942 | 0.880 | 1 |
| IL1R2, OLAH, RETN, MMP8 | | | | | | | | | |
| RSAD2, IFI27, OAS1, ISG15, | 0.873 | 0.864 | 0.882 | 0.924 | 0.873 | 0.975 | 0.933 | 0.860 | 1 |
| HERC6, SLC1A2, IL1R2, RETN | | | | | | | | | |
| RSAD2, IFI27, OAS1, IFIT1, | 0.873 | 0.864 | 0.882 | 0.927 | 0.882 | 0.972 | 0.954 | 0.907 | 1 |
| SIGLEC1, HERC6, SLC1A2, IL1R2 | | | | | | | | | |
| RSAD2, IFI27, OAS1, HERC6, | 0.873 | 0.864 | 0.882 | 0.923 | 0.872 | 0.974 | 0.933 | 0.860 | 1 |
| SLC1A2, IL1R2, RETN, MMP8 | | | | | | | | | |
| RSAD2, IFI27, OAS1, IFIT1, | 0.873 | 0.864 | 0.882 | 0.918 | 0.863 | 0.974 | 0.943 | 0.884 | 1 |
| IFI44L, IL1R2, RETN, MMP8 | | | | | | | | | |
| RSAD2, IFI27, IFIT1, ISG15, | 0.873 | 0.864 | 0.882 | 0.926 | 0.876 | 0.976 | 0.927 | 0.844 | 1 |
| SLC1A2, IL1R2, FAM20A, RETN | | | | | | | | | |
| IFIT1, SIGLEC1, ISG15, HERC6, | 0.873 | 0.863 | 0.883 | 0.920 | 0.865 | 0.975 | 0.948 | 0.877 | 1 |
| SLC1A2, FAM20A, RETN, MMP8 | | | | | | | | | |
| IFIT1, IFI44L, SIGLEC1, HERC6, | 0.873 | 0.863 | 0.882 | 0.926 | 0.878 | 0.974 | 0.941 | 0.869 | 1 |
| SLC1A2, IL1R2, FAM20A, RETN | | | | | | | | | |
| RSAD2, IFI27, OAS1, ISG15, | 0.873 | 0.863 | 0.882 | 0.929 | 0.883 | 0.976 | 0.927 | 0.840 | 1 |
| SLC1A2, IL1R2, FAM20A, MMP8 | | | | | | | | | |
| RSAD2, IFI27, OAS1, IFIT1, ISG15, | 0.873 | 0.864 | 0.882 | 0.926 | 0.873 | 0.979 | 0.935 | 0.865 | 1 |
| SLC1A2, OLAH, RETN | | | | | | | | | |
| RSAD2, IFI27, IFIT1, IFI44L, | 0.873 | 0.864 | 0.881 | 0.927 | 0.875 | 0.979 | 0.935 | 0.868 | 1 |
| ISG15, SLC1A2, OLAH, RETN | | | | | | | | | |
| IFIT1, SIGLEC1, HERC6, SLC1A2, | 0.873 | 0.863 | 0.883 | 0.924 | 0.875 | 0.974 | 0.943 | 0.872 | 1 |
| IL1R2, OLAH, FAM20A, RETN | | | | | | | | | |
| RSAD2, OAS1, IFIT1, SIGLEC1, | 0.873 | 0.863 | 0.882 | 0.911 | 0.847 | 0.974 | 0.964 | 0.916 | 1 |
| IL1R2, OLAH, FAM20A, RETN | | | | | | | | | |
| RSAD2, IFI27, IFIT1, SIGLEC1, | 0.873 | 0.863 | 0.882 | 0.922 | 0.866 | 0.978 | 0.940 | 0.864 | 1 |
| ISG15, SLC1A2, FAM20A, MMP8 | | | | | | | | | |
| RSAD2, IFI27, OAS1, IFI44L, | 0.873 | 0.863 | 0.882 | 0.927 | 0.877 | 0.977 | 0.930 | 0.862 | 0.999 |
| SLC1A2, IL1R2, OLAH, MMP8 | | | | | | | | | |
| RSAD2, IFI27, OAS1, IFI44L, | 0.873 | 0.864 | 0.882 | 0.928 | 0.876 | 0.980 | 0.933 | 0.862 | 1 |
| ISG15, SLC1A2, OLAH, RETN | | | | | | | | | |
| RSAD2, IFI27, IFIT1, SIGLEC1, | 0.873 | 0.864 | 0.881 | 0.927 | 0.879 | 0.974 | 0.964 | 0.922 | 1 |
| SLC1A2, IL1R2, RETN, MMP8 | | | | | | | | | |
| IFI27, OAS1, ISG15, HERC6, | 0.873 | 0.864 | 0.882 | 0.926 | 0.877 | 0.975 | 0.933 | 0.863 | 1 |
| SLC1A2, IL1R2, RETN, MMP8 | | | | | | | | | |
| RSAD2, IFI27, SIGLEC1, ISG15, | 0.873 | 0.864 | 0.881 | 0.926 | 0.878 | 0.973 | 0.951 | 0.901 | 1 |
| HERC6, SLC1A2, IL1R2, RETN | | | | | | | | | |
| RSAD2, IFI27, OAS1, IFI44L, | 0.873 | 0.864 | 0.881 | 0.924 | 0.875 | 0.974 | 0.951 | 0.892 | 1 |
| SIGLEC1, SLC1A2, IL1R2, RETN | | | | | | | | | |
| RSAD2, IFI27, OAS1, IFIT1, | 0.873 | 0.863 | 0.882 | 0.926 | 0.873 | 0.979 | 0.933 | 0.866 | 1.000 |
| IFI44L, ISG15, SLC1A2, OLAH | | | | | | | | | |
| IFIT1, SIGLEC1, ISG15, HERC6, | 0.873 | 0.863 | 0.883 | 0.919 | 0.866 | 0.973 | 0.935 | 0.861 | 1 |
| SLC1A2, OLAH, FAM20A, RETN | | | | | | | | | |
| RSAD2, IFI27, OAS1, IFIT1, | 0.873 | 0.864 | 0.881 | 0.926 | 0.874 | 0.979 | 0.940 | 0.873 | 1 |
| IFI44L, SLC1A2, OLAH, RETN | | | | | | | | | |
| IFI27, IFIT1, SIGLEC1, ISG15, | 0.873 | 0.864 | 0.881 | 0.925 | 0.878 | 0.973 | 0.966 | 0.927 | 1 |
| SLC1A2, IL1R2, RETN, MMP8 | | | | | | | | | |
| RSAD2, OAS1, SIGLEC1, ISG15, | 0.873 | 0.863 | 0.882 | 0.916 | 0.858 | 0.974 | 0.958 | 0.903 | 1 |
| HERC6, IL1R2, OLAH, FAM20A | | | | | | | | | |
| OAS1, IFIT1, IFI44L, SIGLEC1, | 0.873 | 0.863 | 0.882 | 0.919 | 0.864 | 0.975 | 0.934 | 0.868 | 0.999 |
| ISG15, SLC1A2, OLAH, FAM20A | | | | | | | | | |
| RSAD2, IFI27, OAS1, IFIT1, | 0.872 | 0.863 | 0.882 | 0.922 | 0.867 | 0.978 | 0.941 | 0.867 | 1 |
| SIGLEC1, ISG15, SLC1A2, FAM20A | | | | | | | | | |
| IFI27, OAS1, IFI44L, SIGLEC1, | 0.872 | 0.864 | 0.881 | 0.926 | 0.878 | 0.974 | 0.948 | 0.893 | 1 |
| ISG15, SLC1A2, IL1R2, RETN | | | | | | | | | |
| RSAD2, IFI27, OAS1, SIGLEC1, | 0.872 | 0.863 | 0.882 | 0.926 | 0.880 | 0.971 | 0.950 | 0.899 | 1 |
| HERC6, SLC1A2, IL1R2, MMP8 | | | | | | | | | |
| IFI27, OAS1, IFIT1, IFI44L, | 0.872 | 0.863 | 0.882 | 0.926 | 0.874 | 0.977 | 0.937 | 0.869 | 1 |
| SLC1A2, IL1R2, OLAH, RETN | | | | | | | | | |
| IFIT1, IFI44L, SIGLEC1, SLC1A2, | 0.872 | 0.863 | 0.882 | 0.921 | 0.868 | 0.974 | 0.946 | 0.885 | 1 |
| IL1R2, OLAH, FAM20A, RETN | | | | | | | | | |
| RSAD2, IFI27, OAS1, IFIT1, | 0.872 | 0.863 | 0.882 | 0.927 | 0.877 | 0.977 | 0.920 | 0.831 | 1 |
| SLC1A2, IL1R2, FAM20A, RETN | | | | | | | | | |
| RSAD2, IFI27, OAS1, IFI44L, | 0.872 | 0.864 | 0.881 | 0.920 | 0.870 | 0.971 | 0.949 | 0.902 | 0.996 |
| SIGLEC1, ISG15, IL1R2, MMP8 | | | | | | | | | |
| SIGLEC1, HERC6, SLC1A2, IL1R2, | 0.872 | 0.862 | 0.883 | 0.923 | 0.872 | 0.973 | 0.958 | 0.892 | 1 |
| OLAH, FAM20A, RETN, MMP8 | | | | | | | | | |
| IFI27, OAS1, IFI44L, ISG15, | 0.872 | 0.863 | 0.882 | 0.928 | 0.878 | 0.977 | 0.925 | 0.855 | 0.995 |
| SLC1A2, IL1R2, OLAH, MMP8 | | | | | | | | | |
| RSAD2, IFI44L, SIGLEC1, ISG15, | 0.872 | 0.863 | 0.882 | 0.925 | 0.878 | 0.973 | 0.959 | 0.893 | 1 |
| SLC1A2, IL1R2, FAM20A, MMP8 | | | | | | | | | |
| OAS1, IFIT1, SIGLEC1, HERC6, | 0.872 | 0.863 | 0.882 | 0.924 | 0.876 | 0.973 | 0.945 | 0.873 | 1 |
| SLC1A2, IL1R2, FAM20A, RETN | | | | | | | | | |
| OAS1, IFIT1, IFI44L, SIGLEC1, | 0.872 | 0.863 | 0.882 | 0.910 | 0.847 | 0.972 | 0.963 | 0.911 | 1 |
| ISG15, IL1R2, FAM20A, RETN | | | | | | | | | |
| RSAD2, IFI27, IFIT1, IFI44L, | 0.872 | 0.864 | 0.880 | 0.919 | 0.869 | 0.969 | 0.943 | 0.893 | 0.994 |
| SIGLEC1, ISG15, IL1R2, MMP8 | | | | | | | | | |
| IFI27, OAS1, SIGLEC1, ISG15, | 0.872 | 0.864 | 0.881 | 0.927 | 0.880 | 0.974 | 0.963 | 0.916 | 1 |
| SLC1A2, IL1R2, RETN, MMP8 | | | | | | | | | |
| RSAD2, IFI27, OAS1, SIGLEC1, | 0.872 | 0.863 | 0.881 | 0.925 | 0.877 | 0.973 | 0.958 | 0.903 | 1 |
| SLC1A2, IL1R2, RETN, MMP8 | | | | | | | | | |
| RSAD2, IFI27, IFI44L, SIGLEC1, | 0.872 | 0.864 | 0.881 | 0.928 | 0.880 | 0.975 | 0.962 | 0.921 | 1 |
| SLC1A2, IL1R2, RETN, MMP8 | | | | | | | | | |
| RSAD2, IFI27, IFIT1, IFI44L, | 0.872 | 0.863 | 0.882 | 0.922 | 0.866 | 0.977 | 0.929 | 0.846 | 1 |
| HERC6, SLC1A2, FAM20A, RETN | | | | | | | | | |
| RSAD2, OAS1, SIGLEC1, SLC1A2, | 0.872 | 0.862 | 0.882 | 0.924 | 0.874 | 0.973 | 0.958 | 0.894 | 1 |
| IL1R2, FAM20A, RETN, MMP8 | | | | | | | | | |
| OAS1, IFI44L, SIGLEC1, ISG15, | 0.872 | 0.863 | 0.882 | 0.924 | 0.876 | 0.972 | 0.959 | 0.893 | 1 |
| SLC1A2, IL1R2, FAM20A, MMP8 | | | | | | | | | |
| RSAD2, IFI27, OAS1, SIGLEC1, | 0.872 | 0.864 | 0.881 | 0.923 | 0.873 | 0.972 | 0.955 | 0.898 | 1 |
| ISG15, SLC1A2, IL1R2, RETN | | | | | | | | | |
| IFI27, OAS1, ISG15, HERC6, | 0.872 | 0.862 | 0.883 | 0.922 | 0.866 | 0.979 | 0.934 | 0.853 | 1 |
| SLC1A2, FAM20A, RETN, MMP8 | | | | | | | | | |
| RSAD2, IFI27, OAS1, IFI44L, | 0.872 | 0.863 | 0.881 | 0.926 | 0.880 | 0.972 | 0.955 | 0.909 | 1 |
| SIGLEC1, HERC6, SLC1A2, IL1R2 | | | | | | | | | |
| RSAD2, IFI27, IFI44L, ISG15, | 0.872 | 0.862 | 0.882 | 0.927 | 0.878 | 0.977 | 0.923 | 0.836 | 1 |
| SLC1A2, IL1R2, FAM20A, RETN | | | | | | | | | |
| RSAD2, IFI27, OAS1, SIGLEC1, | 0.872 | 0.862 | 0.883 | 0.919 | 0.865 | 0.974 | 0.939 | 0.863 | 1 |
| HERC6, SLC1A2, FAM20A, MMP8 | | | | | | | | | |
| RSAD2, IFI27, OAS1, IFI44L, | 0.872 | 0.862 | 0.882 | 0.925 | 0.871 | 0.980 | 0.940 | 0.875 | 1 |
| SLC1A2, OLAH, RETN, MMP8 | | | | | | | | | |
| OAS1, IFIT1, SIGLEC1, HERC6, | 0.872 | 0.862 | 0.882 | 0.921 | 0.869 | 0.973 | 0.936 | 0.865 | 1 |
| SLC1A2, OLAH, FAM20A, RETN | | | | | | | | | |
| RSAD2, OAS1, IFIT1, IFI44L, | 0.872 | 0.863 | 0.881 | 0.911 | 0.850 | 0.972 | 0.935 | 0.866 | 1 |
| SIGLEC1, ISG15, HERC6, FAM20A | | | | | | | | | |
| OAS1, IFIT1, IFI44L, SIGLEC1, | 0.872 | 0.863 | 0.881 | 0.925 | 0.875 | 0.975 | 0.935 | 0.862 | 1 |
| HERC6, SLC1A2, FAM20A, RETN | | | | | | | | | |
| RSAD2, IFI44L, SIGLEC1, IL1R2, | 0.872 | 0.862 | 0.882 | 0.919 | 0.862 | 0.976 | 0.964 | 0.911 | 1 |
| OLAH, FAM20A, RETN, MMP8 | | | | | | | | | |
| IFIT1, IFI44L, SIGLEC1, ISG15, | 0.872 | 0.863 | 0.881 | 0.923 | 0.872 | 0.975 | 0.935 | 0.859 | 1 |
| HERC6, SLC1A2, FAM20A, RETN | | | | | | | | | |
| RSAD2, IFI44L, SIGLEC1, ISG15, | 0.872 | 0.862 | 0.882 | 0.915 | 0.856 | 0.974 | 0.958 | 0.901 | 1 |
| HERC6, IL1R2, OLAH, FAM20A | | | | | | | | | |
| RSAD2, IFI27, OAS1, IFIT1, | 0.872 | 0.863 | 0.881 | 0.926 | 0.875 | 0.977 | 0.938 | 0.872 | 1 |
| IFI44L, SLC1A2, IL1R2, OLAH | | | | | | | | | |
| RSAD2, IFI27, IFIT1, IFI44L, | 0.872 | 0.863 | 0.881 | 0.922 | 0.869 | 0.975 | 0.935 | 0.869 | 1 |
| SLC1A2, IL1R2, OLAH, RETN | | | | | | | | | |
| RSAD2, IFI27, OAS1, IFIT1, ISG15, | 0.872 | 0.863 | 0.882 | 0.925 | 0.876 | 0.974 | 0.923 | 0.836 | 1 |
| SLC1A2, IL1R2, FAM20A | | | | | | | | | |
| RSAD2, IFIT1, IFI44L, SIGLEC1, | 0.872 | 0.862 | 0.882 | 0.907 | 0.841 | 0.974 | 0.954 | 0.903 | 1 |
| ISG15, OLAH, FAM20A, MMP8 | | | | | | | | | |
| OAS1, IFIT1, IFI44L, SIGLEC1, | 0.872 | 0.863 | 0.882 | 0.921 | 0.868 | 0.974 | 0.946 | 0.885 | 1 |
| SLC1A2, IL1R2, OLAH, FAM20A | | | | | | | | | |
| IFI27, OAS1, IFIT1, IFI44L, | 0.872 | 0.863 | 0.881 | 0.925 | 0.880 | 0.970 | 0.949 | 0.901 | 0.997 |
| SIGLEC1, HERC6, SLC1A2, IL1R2 | | | | | | | | | |
| RSAD2, IFI27, OAS1, IFI44L, | 0.872 | 0.863 | 0.881 | 0.926 | 0.874 | 0.979 | 0.936 | 0.866 | 1 |
| SLC1A2, IL1R2, OLAH, RETN | | | | | | | | | |
| RSAD2, IFI27, IFIT1, IFI44L, | 0.872 | 0.863 | 0.881 | 0.928 | 0.878 | 0.977 | 0.929 | 0.864 | 0.995 |
| ISG15, SLC1A2, IL1R2, OLAH | | | | | | | | | |
| IFI27, OAS1, IFIT1, SIGLEC1, | 0.872 | 0.863 | 0.881 | 0.926 | 0.882 | 0.971 | 0.951 | 0.902 | 1 |
| ISG15, HERC6, SLC1A2, IL1R2 | | | | | | | | | |
| RSAD2, IFI27, OAS1, IFI44L, | 0.872 | 0.862 | 0.882 | 0.924 | 0.868 | 0.980 | 0.935 | 0.853 | 1 |
| HERC6, SLC1A2, FAM20A, RETN | | | | | | | | | |
| IFI27, OAS1, SIGLEC1, ISG15, | 0.872 | 0.863 | 0.881 | 0.926 | 0.882 | 0.971 | 0.948 | 0.897 | 0.999 |
| HERC6, SLC1A2, IL1R2, MMP8 | | | | | | | | | |
| RSAD2, OAS1, SIGLEC1, ISG15, | 0.872 | 0.862 | 0.882 | 0.919 | 0.866 | 0.973 | 0.951 | 0.889 | 1 |
| SLC1A2, OLAH, FAM20A, MMP8 | | | | | | | | | |
| RSAD2, IFIT1, SIGLEC1, ISG15, | 0.872 | 0.863 | 0.881 | 0.912 | 0.850 | 0.974 | 0.964 | 0.916 | 1 |
| IL1R2, OLAH, FAM20A, RETN | | | | | | | | | |
| RSAD2, IFIT1, SIGLEC1, HERC6, | 0.872 | 0.862 | 0.882 | 0.920 | 0.867 | 0.974 | 0.938 | 0.865 | 1 |
| SLC1A2, OLAH, FAM20A, RETN | | | | | | | | | |
| RSAD2, IFI27, OAS1, IFI44L, | 0.872 | 0.862 | 0.882 | 0.921 | 0.867 | 0.975 | 0.941 | 0.866 | 1 |
| SIGLEC1, HERC6, SLC1A2, FAM20A | | | | | | | | | |
| RSAD2, IFI27, OAS1, SIGLEC1, | 0.872 | 0.863 | 0.881 | 0.926 | 0.881 | 0.971 | 0.953 | 0.905 | 1 |
| ISG15, HERC6, SLC1A2, IL1R2 | | | | | | | | | |
| OAS1, IFIT1, SIGLEC1, SLC1A2, | 0.872 | 0.862 | 0.881 | 0.920 | 0.867 | 0.973 | 0.954 | 0.895 | 1 |
| IL1R2, OLAH, FAM20A, RETN | | | | | | | | | |
| IFIT1, SIGLEC1, ISG15, SLC1A2, | 0.872 | 0.862 | 0.882 | 0.920 | 0.867 | 0.974 | 0.950 | 0.890 | 1 |
| OLAH, FAM20A, RETN, MMP8 | | | | | | | | | |
| RSAD2, IFI27, IFIT1, ISG15, | 0.872 | 0.862 | 0.882 | 0.922 | 0.867 | 0.977 | 0.934 | 0.852 | 1 |
| HERC6, SLC1A2, FAM20A, RETN | | | | | | | | | |
| RSAD2, IFI27, OAS1, IFIT1, | 0.872 | 0.862 | 0.882 | 0.921 | 0.864 | 0.978 | 0.942 | 0.870 | 1 |
| SIGLEC1, SLC1A2, FAM20A, MMP8 | | | | | | | | | |
| RSAD2, IFI44L, SIGLEC1, ISG15, | 0.872 | 0.862 | 0.882 | 0.918 | 0.860 | 0.976 | 0.963 | 0.908 | 1 |
| IL1R2, FAM20A, RETN, MMP8 | | | | | | | | | |
| RSAD2, IFI27, OAS1, IFIT1, ISG15, | 0.872 | 0.863 | 0.880 | 0.918 | 0.870 | 0.967 | 0.941 | 0.888 | 0.994 |
| HERC6, IL1R2, MMP8 | | | | | | | | | |
| OAS1, IFIT1, SIGLEC1, SLC1A2, | 0.872 | 0.862 | 0.882 | 0.923 | 0.870 | 0.976 | 0.953 | 0.896 | 1 |
| OLAH, FAM20A, RETN, MMP8 | | | | | | | | | |
| RSAD2, OAS1, IFIT1, IFI44L, | 0.872 | 0.862 | 0.881 | 0.920 | 0.865 | 0.975 | 0.939 | 0.876 | 1 |
| SIGLEC1, SLC1A2, OLAH, FAM20A | | | | | | | | | |
| RSAD2, IFI27, OAS1, IFI44L, | 0.872 | 0.861 | 0.883 | 0.915 | 0.851 | 0.978 | 0.943 | 0.871 | 1 |
| SIGLEC1, ISG15, FAM20A, MMP8 | | | | | | | | | |
| IFI27, OAS1, IFI44L, ISG15, | 0.872 | 0.862 | 0.881 | 0.929 | 0.878 | 0.979 | 0.932 | 0.863 | 1 |
| SLC1A2, IL1R2, OLAH, RETN | | | | | | | | | |
| RSAD2, IFIT1, SIGLEC1, HERC6, | 0.872 | 0.862 | 0.881 | 0.925 | 0.876 | 0.974 | 0.946 | 0.874 | 1 |
| SLC1A2, IL1R2, FAM20A, RETN | | | | | | | | | |
| RSAD2, OAS1, IFI44L, SIGLEC1, | 0.872 | 0.862 | 0.881 | 0.915 | 0.856 | 0.974 | 0.958 | 0.904 | 1 |
| ISG15, HERC6, IL1R2, FAM20A | | | | | | | | | |
| RSAD2, OAS1, IFIT1, SIGLEC1, | 0.872 | 0.862 | 0.881 | 0.914 | 0.851 | 0.977 | 0.961 | 0.913 | 1 |
| OLAH, FAM20A, RETN, MMP8 | | | | | | | | | |
| RSAD2, IFI27, OAS1, IFIT1, | 0.872 | 0.863 | 0.880 | 0.919 | 0.870 | 0.969 | 0.937 | 0.881 | 0.993 |
| IFI44L, ISG15, HERC6, IL1R2 | | | | | | | | | |
| RSAD2, SIGLEC1, ISG15, HERC6, | 0.872 | 0.862 | 0.882 | 0.921 | 0.869 | 0.972 | 0.946 | 0.877 | 1 |
| SLC1A2, OLAH, FAM20A, MMP8 | | | | | | | | | |
| RSAD2, IFIT1, SIGLEC1, HERC6, | 0.872 | 0.862 | 0.881 | 0.920 | 0.866 | 0.975 | 0.942 | 0.870 | 1 |
| SLC1A2, FAM20A, RETN, MMP8 | | | | | | | | | |
| IFI27, IFIT1, IFI44L, HERC6, | 0.872 | 0.861 | 0.882 | 0.922 | 0.864 | 0.979 | 0.935 | 0.852 | 1 |
| SLC1A2, FAM20A, RETN, MMP8 | | | | | | | | | |
| OAS1, SIGLEC1, ISG15, HERC6, | 0.872 | 0.861 | 0.882 | 0.919 | 0.866 | 0.971 | 0.942 | 0.874 | 1 |
| SLC1A2, OLAH, FAM20A, MMP8 | | | | | | | | | |
| RSAD2, IFIT1, IFI44L, SIGLEC1, | 0.872 | 0.862 | 0.881 | 0.920 | 0.866 | 0.975 | 0.939 | 0.876 | 1 |
| ISG15, SLC1A2, OLAH, FAM20A | | | | | | | | | |
| RSAD2, OAS1, IFI44L, SIGLEC1, | 0.872 | 0.861 | 0.882 | 0.918 | 0.863 | 0.973 | 0.947 | 0.883 | 1 |
| SLC1A2, OLAH, FAM20A, MMP8 | | | | | | | | | |
| RSAD2, OAS1, IFI44L, SIGLEC1, | 0.872 | 0.862 | 0.881 | 0.914 | 0.856 | 0.973 | 0.958 | 0.904 | 1 |
| HERC6, IL1R2, OLAH, FAM20A | | | | | | | | | |
| IFIT1, IFI44L, SIGLEC1, ISG15, | 0.872 | 0.862 | 0.881 | 0.920 | 0.867 | 0.973 | 0.934 | 0.856 | 1 |
| HERC6, SLC1A2, FAM20A, MMP8 | | | | | | | | | |
| IFI27, OAS1, IFI44L, SIGLEC1, | 0.872 | 0.862 | 0.881 | 0.922 | 0.869 | 0.975 | 0.942 | 0.866 | 1 |
| HERC6, SLC1A2, FAM20A, MMP8 | | | | | | | | | |
| OAS1, SIGLEC1, HERC6, SLC1A2, | 0.872 | 0.861 | 0.882 | 0.918 | 0.862 | 0.973 | 0.947 | 0.882 | 1 |
| OLAH, FAM20A, RETN, MMP8 | | | | | | | | | |
| IFI27, OAS1, IFI44L, ISG15, | 0.872 | 0.863 | 0.880 | 0.924 | 0.874 | 0.973 | 0.936 | 0.869 | 1 |
| HERC6, SLC1A2, IL1R2, RETN | | | | | | | | | |
| RSAD2, SIGLEC1, HERC6, | 0.872 | 0.861 | 0.882 | 0.920 | 0.867 | 0.974 | 0.945 | 0.874 | 1 |
| SLC1A2, OLAH, FAM20A, RETN, MMP8 | | | | | | | | | |
| IFI27, OAS1, IFIT1, IFI44L, ISG15, | 0.872 | 0.862 | 0.881 | 0.919 | 0.864 | 0.975 | 0.945 | 0.889 | 1 |
| IL1R2, RETN, MMP8 | | | | | | | | | |
| IFI27, OAS1, IFIT1, IFI44L, ISG15, | 0.872 | 0.862 | 0.881 | 0.915 | 0.857 | 0.973 | 0.932 | 0.857 | 1 |
| HERC6, SLC1A2, RETN | | | | | | | | | |
| RSAD2, IFI27, OAS1, IFIT1, | 0.872 | 0.862 | 0.881 | 0.923 | 0.867 | 0.979 | 0.933 | 0.849 | 1 |
| HERC6, SLC1A2, FAM20A, RETN | | | | | | | | | |
| RSAD2, OAS1, IFIT1, SIGLEC1, | 0.872 | 0.862 | 0.881 | 0.911 | 0.847 | 0.974 | 0.962 | 0.910 | 1 |
| ISG15, IL1R2, FAM20A, RETN | | | | | | | | | |
| RSAD2, IFI27, SIGLEC1, ISG15, | 0.871 | 0.861 | 0.882 | 0.921 | 0.867 | 0.976 | 0.939 | 0.863 | 1 |
| HERC6, SLC1A2, FAM20A, MMP8 | | | | | | | | | |
| OAS1, IFIT1, IFI44L, SIGLEC1, | 0.871 | 0.862 | 0.881 | 0.920 | 0.864 | 0.975 | 0.937 | 0.874 | 1 |
| SLC1A2, OLAH, FAM20A, RETN | | | | | | | | | |
| IFI27, OAS1, IFI44L, HERC6, | 0.871 | 0.861 | 0.882 | 0.923 | 0.866 | 0.981 | 0.937 | 0.856 | 1 |
| SLC1A2, FAM20A, RETN, MMP8 | | | | | | | | | |
| OAS1, IFI44L, SIGLEC1, ISG15, | 0.871 | 0.862 | 0.881 | 0.914 | 0.856 | 0.973 | 0.959 | 0.905 | 1 |
| HERC6, IL1R2, OLAH, FAM20A | | | | | | | | | |
| OAS1, IFIT1, IFI44L, SIGLEC1, | 0.871 | 0.862 | 0.881 | 0.920 | 0.867 | 0.972 | 0.934 | 0.857 | 1 |
| HERC6, SLC1A2, FAM20A, MMP8 | | | | | | | | | |
| RSAD2, IFI27, OAS1, SIGLEC1, | 0.871 | 0.861 | 0.882 | 0.920 | 0.866 | 0.974 | 0.940 | 0.864 | 1 |
| ISG15, HERC6, SLC1A2, FAM20A | | | | | | | | | |
| RSAD2, OAS1, IFIT1, SIGLEC1, | 0.871 | 0.862 | 0.881 | 0.919 | 0.865 | 0.973 | 0.955 | 0.897 | 1 |
| SLC1A2, IL1R2, OLAH, FAM20A | | | | | | | | | |
| RSAD2, IFI27, OAS1, IFIT1, ISG15, | 0.871 | 0.862 | 0.881 | 0.915 | 0.856 | 0.975 | 0.928 | 0.850 | 1 |
| HERC6, SLC1A2, RETN | | | | | | | | | |
| RSAD2, IFI27, OAS1, ISG15, | 0.871 | 0.862 | 0.881 | 0.926 | 0.877 | 0.975 | 0.921 | 0.833 | 1 |
| SLC1A2, IL1R2, FAM20A, RETN | | | | | | | | | |
| RSAD2, IFI27, OAS1, IFI44L, | 0.871 | 0.862 | 0.881 | 0.924 | 0.873 | 0.976 | 0.924 | 0.838 | 1 |
| SLC1A2, IL1R2, FAM20A, RETN | | | | | | | | | |
| RSAD2, IFI27, SIGLEC1, ISG15, | 0.871 | 0.863 | 0.880 | 0.927 | 0.880 | 0.974 | 0.963 | 0.920 | 1 |
| SLC1A2, IL1R2, RETN, MMP8 | | | | | | | | | |
| IFI27, OAS1, IFIT1, IFI44L, ISG15, | 0.871 | 0.862 | 0.881 | 0.927 | 0.878 | 0.977 | 0.929 | 0.863 | 0.996 |
| SLC1A2, IL1R2, OLAH | | | | | | | | | |
| IFI27, OAS1, IFIT1, IFI44L, | 0.871 | 0.862 | 0.880 | 0.924 | 0.874 | 0.973 | 0.938 | 0.873 | 1 |
| HERC6, SLC1A2, IL1R2, RETN | | | | | | | | | |
| IFI27, OAS1, IFI44L, SIGLEC1, | 0.871 | 0.862 | 0.880 | 0.926 | 0.882 | 0.970 | 0.948 | 0.899 | 0.997 |
| HERC6, SLC1A2, IL1R2, MMP8 | | | | | | | | | |
| RSAD2, IFIT1, IFI44L, SIGLEC1, | 0.871 | 0.862 | 0.881 | 0.922 | 0.869 | 0.974 | 0.950 | 0.892 | 1 |
| SLC1A2, IL1R2, OLAH, FAM20A | | | | | | | | | |
| RSAD2, IFI27, OAS1, IFI44L, | 0.871 | 0.863 | 0.880 | 0.922 | 0.875 | 0.969 | 0.932 | 0.873 | 0.990 |
| ISG15, HERC6, IL1R2, MMP8 | | | | | | | | | |
| RSAD2, IFI44L, SIGLEC1, | 0.871 | 0.861 | 0.881 | 0.924 | 0.874 | 0.973 | 0.958 | 0.894 | 1 |
| SLC1A2, IL1R2, FAM20A, RETN, MMP8 | | | | | | | | | |
| OAS1, IFI44L, SIGLEC1, SLC1A2, | 0.871 | 0.861 | 0.881 | 0.923 | 0.873 | 0.973 | 0.958 | 0.894 | 1 |
| IL1R2, FAM20A, RETN, MMP8 | | | | | | | | | |
| OAS1, IFI44L, SIGLEC1, ISG15, | 0.871 | 0.861 | 0.881 | 0.917 | 0.861 | 0.972 | 0.946 | 0.882 | 1 |
| SLC1A2, OLAH, FAM20A, MMP8 | | | | | | | | | |
| IFI27, OAS1, IFI44L, HERC6, | 0.871 | 0.862 | 0.880 | 0.924 | 0.875 | 0.974 | 0.935 | 0.867 | 1 |
| SLC1A2, IL1R2, RETN, MMP8 | | | | | | | | | |
| IFI27, IFIT1, IFI44L, SIGLEC1, | 0.871 | 0.863 | 0.879 | 0.925 | 0.880 | 0.970 | 0.947 | 0.897 | 0.997 |
| ISG15, SLC1A2, IL1R2, MMP8 | | | | | | | | | |
| RSAD2, IFIT1, IFI44L, SIGLEC1, | 0.871 | 0.862 | 0.881 | 0.912 | 0.848 | 0.975 | 0.964 | 0.916 | 1 |
| IL1R2, OLAH, FAM20A, RETN | | | | | | | | | |
| IFI44L, SIGLEC1, ISG15, HERC6, | 0.871 | 0.861 | 0.881 | 0.919 | 0.865 | 0.973 | 0.946 | 0.873 | 1 |
| SLC1A2, OLAH, FAM20A, MMP8 | | | | | | | | | |
| IFI27, OAS1, SIGLEC1, ISG15, | 0.871 | 0.861 | 0.882 | 0.920 | 0.866 | 0.974 | 0.941 | 0.865 | 1 |
| HERC6, SLC1A2, FAM20A, MMP8 | | | | | | | | | |
| RSAD2, OAS1, IFIT1, IFI44L, | 0.871 | 0.862 | 0.880 | 0.921 | 0.869 | 0.973 | 0.933 | 0.856 | 1 |
| SIGLEC1, HERC6, SLC1A2, FAM20A | | | | | | | | | |
| RSAD2, IFI27, IFI44L, SIGLEC1, | 0.871 | 0.861 | 0.881 | 0.921 | 0.867 | 0.976 | 0.940 | 0.864 | 1 |
| HERC6, SLC1A2, FAM20A, MMP8 | | | | | | | | | |
| RSAD2, IFI27, IFIT1, IFI44L, | 0.871 | 0.863 | 0.879 | 0.922 | 0.870 | 0.975 | 0.936 | 0.875 | 0.997 |
| SIGLEC1, HERC6, SLC1A2, RETN | | | | | | | | | |
| RSAD2, IFI27, OAS1, IFIT1, | 0.871 | 0.862 | 0.880 | 0.915 | 0.860 | 0.970 | 0.942 | 0.884 | 1 |
| IFI44L, ISG15, IL1R2, RETN | | | | | | | | | |
| RSAD2, IFIT1, SIGLEC1, SLC1A2, | 0.871 | 0.862 | 0.881 | 0.921 | 0.868 | 0.974 | 0.957 | 0.898 | 1 |
| IL1R2, OLAH, FAM20A, RETN | | | | | | | | | |
| OAS1, IFIT1, SIGLEC1, ISG15, | 0.871 | 0.861 | 0.881 | 0.918 | 0.864 | 0.973 | 0.952 | 0.891 | 1 |
| SLC1A2, IL1R2, OLAH, FAM20A | | | | | | | | | |
| RSAD2, IFI27, IFI44L, SIGLEC1, | 0.871 | 0.863 | 0.879 | 0.925 | 0.875 | 0.975 | 0.948 | 0.896 | 1 |
| ISG15, SLC1A2, IL1R2, RETN | | | | | | | | | |
| RSAD2, IFI27, IFIT1, IFI44L, | 0.871 | 0.861 | 0.881 | 0.914 | 0.855 | 0.974 | 0.916 | 0.835 | 0.997 |
| ISG15, HERC6, FAM20A, MMP8 | | | | | | | | | |
| IFI44L, SIGLEC1, ISG15, HERC6, | 0.871 | 0.861 | 0.881 | 0.911 | 0.850 | 0.972 | 0.958 | 0.899 | 1 |
| IL1R2, OLAH, FAM20A, RETN | | | | | | | | | |
| RSAD2, IFIT1, IFI44L, SIGLEC1, | 0.871 | 0.861 | 0.880 | 0.911 | 0.850 | 0.972 | 0.935 | 0.866 | 1 |
| ISG15, HERC6, FAM20A, MMP8 | | | | | | | | | |
| RSAD2, OAS1, IFIT1, SIGLEC1, | 0.871 | 0.861 | 0.880 | 0.910 | 0.849 | 0.971 | 0.939 | 0.870 | 1 |
| ISG15, HERC6, FAM20A, MMP8 | | | | | | | | | |
| RSAD2, IFI27, IFI44L, SIGLEC1, | 0.871 | 0.861 | 0.881 | 0.922 | 0.867 | 0.976 | 0.941 | 0.864 | 1 |
| ISG15, HERC6, SLC1A2, FAM20A | | | | | | | | | |
| RSAD2, IFI27, IFIT1, IFI44L, | 0.871 | 0.862 | 0.879 | 0.923 | 0.873 | 0.974 | 0.949 | 0.896 | 1 |
| SIGLEC1, SLC1A2, IL1R2, RETN | | | | | | | | | |
| RSAD2, IFI27, ISG15, HERC6, | 0.871 | 0.860 | 0.881 | 0.925 | 0.870 | 0.980 | 0.934 | 0.852 | 1 |
| SLC1A2, FAM20A, RETN, MMP8 | | | | | | | | | |
| RSAD2, OAS1, SIGLEC1, ISG15, | 0.871 | 0.861 | 0.880 | 0.913 | 0.852 | 0.974 | 0.951 | 0.888 | 1 |
| HERC6, FAM20A, RETN, MMP8 | | | | | | | | | |
| RSAD2, OAS1, IFIT1, IFI44L, | 0.871 | 0.861 | 0.880 | 0.905 | 0.836 | 0.975 | 0.947 | 0.891 | 1 |
| SIGLEC1, ISG15, OLAH, FAM20A | | | | | | | | | |
| RSAD2, IFIT1, IFI44L, SIGLEC1, | 0.871 | 0.861 | 0.880 | 0.920 | 0.868 | 0.972 | 0.929 | 0.852 | 1 |
| ISG15, HERC6, SLC1A2, FAM20A | | | | | | | | | |
| IFI27, SIGLEC1, ISG15, HERC6, | 0.871 | 0.862 | 0.879 | 0.927 | 0.879 | 0.974 | 0.965 | 0.926 | 1 |
| SLC1A2, IL1R2, RETN, MMP8 | | | | | | | | | |
| RSAD2, IFI44L, SIGLEC1, ISG15, | 0.871 | 0.861 | 0.880 | 0.918 | 0.862 | 0.973 | 0.943 | 0.879 | 1 |
| SLC1A2, OLAH, FAM20A, MMP8 | | | | | | | | | |
| RSAD2, IFI27, IFIT1, SIGLEC1, | 0.871 | 0.862 | 0.879 | 0.926 | 0.880 | 0.971 | 0.950 | 0.902 | 0.998 |
| HERC6, SLC1A2, IL1R2, MMP8 | | | | | | | | | |
| RSAD2, OAS1, SIGLEC1, HERC6, | 0.871 | 0.861 | 0.880 | 0.916 | 0.856 | 0.976 | 0.951 | 0.893 | 1 |
| OLAH, FAM20A, RETN, MMP8 | | | | | | | | | |
| OAS1, IFIT1, IFI44L, SIGLEC1, | 0.871 | 0.861 | 0.880 | 0.904 | 0.834 | 0.974 | 0.947 | 0.891 | 1 |
| ISG15, OLAH, FAM20A, RETN | | | | | | | | | |
| RSAD2, IFIT1, SIGLEC1, ISG15, | 0.871 | 0.861 | 0.880 | 0.918 | 0.864 | 0.973 | 0.957 | 0.895 | 1 |
| SLC1A2, IL1R2, OLAH, FAM20A | | | | | | | | | |
| OAS1, IFIT1, SIGLEC1, ISG15, | 0.871 | 0.861 | 0.880 | 0.920 | 0.868 | 0.972 | 0.938 | 0.865 | 1 |
| HERC6, SLC1A2, FAM20A, RETN | | | | | | | | | |
| IFI27, OAS1, IFI44L, SIGLEC1, | 0.870 | 0.862 | 0.879 | 0.926 | 0.882 | 0.970 | 0.949 | 0.901 | 0.997 |
| ISG15, HERC6, SLC1A2, IL1R2 | | | | | | | | | |
| RSAD2, IFI27, OAS1, IFI44L, | 0.870 | 0.861 | 0.880 | 0.917 | 0.858 | 0.976 | 0.930 | 0.853 | 1 |
| ISG15, HERC6, SLC1A2, RETN | | | | | | | | | |
| RSAD2, IFIT1, IFI44L, SIGLEC1, | 0.870 | 0.861 | 0.880 | 0.920 | 0.864 | 0.976 | 0.938 | 0.875 | 1 |
| SLC1A2, OLAH, FAM20A, RETN | | | | | | | | | |
| IFI27, OAS1, IFIT1, IFI44L, | 0.870 | 0.862 | 0.879 | 0.925 | 0.880 | 0.971 | 0.951 | 0.902 | 1.000 |
| SIGLEC1, SLC1A2, IL1R2, MMP8 | | | | | | | | | |
| RSAD2, IFI27, OAS1, IFIT1, ISG15, | 0.870 | 0.861 | 0.880 | 0.919 | 0.865 | 0.974 | 0.952 | 0.900 | 1 |
| IL1R2, RETN, MMP8 | | | | | | | | | |
| IFIT1, IFI44L, SIGLEC1, ISG15, | 0.870 | 0.861 | 0.879 | 0.919 | 0.867 | 0.971 | 0.950 | 0.891 | 1 |
| SLC1A2, IL1R2, FAM20A, RETN | | | | | | | | | |
| RSAD2, OAS1, IFIT1, IFI44L, | 0.870 | 0.861 | 0.880 | 0.911 | 0.847 | 0.974 | 0.963 | 0.911 | 1 |
| SIGLEC1, IL1R2, FAM20A, RETN | | | | | | | | | |
| RSAD2, IFI44L, SIGLEC1, HERC6, | 0.870 | 0.860 | 0.881 | 0.921 | 0.868 | 0.974 | 0.943 | 0.876 | 1 |
| SLC1A2, OLAH, FAM20A, MMP8 | | | | | | | | | |
| IFI27, OAS1, IFIT1, IFI44L, | 0.870 | 0.862 | 0.879 | 0.924 | 0.878 | 0.970 | 0.945 | 0.893 | 0.996 |
| SIGLEC1, ISG15, SLC1A2, IL1R2 | | | | | | | | | |
| RSAD2, IFI27, IFI44L, ISG15, | 0.870 | 0.860 | 0.881 | 0.923 | 0.868 | 0.978 | 0.936 | 0.856 | 1 |
| HERC6, SLC1A2, FAM20A, RETN | | | | | | | | | |
| OAS1, IFIT1, IFI44L, SIGLEC1, | 0.870 | 0.861 | 0.880 | 0.919 | 0.867 | 0.972 | 0.950 | 0.890 | 1 |
| ISG15, SLC1A2, IL1R2, FAM20A | | | | | | | | | |
| RSAD2, IFI27, IFIT1, SIGLEC1, | 0.870 | 0.862 | 0.879 | 0.926 | 0.878 | 0.974 | 0.953 | 0.903 | 1 |
| ISG15, SLC1A2, IL1R2, RETN | | | | | | | | | |
| OAS1, IFI44L, SIGLEC1, HERC6, | 0.870 | 0.860 | 0.881 | 0.918 | 0.864 | 0.971 | 0.945 | 0.879 | 1 |
| SLC1A2, OLAH, FAM20A, MMP8 | | | | | | | | | |
| IFI44L, SIGLEC1, ISG15, HERC6, | 0.870 | 0.860 | 0.880 | 0.918 | 0.864 | 0.972 | 0.950 | 0.877 | 1 |
| SLC1A2, IL1R2, OLAH, FAM20A | | | | | | | | | |
| IFI27, OAS1, IFIT1, ISG15, HERC6, | 0.870 | 0.860 | 0.880 | 0.915 | 0.857 | 0.973 | 0.932 | 0.856 | 1 |
| SLC1A2, RETN, MMP8 | | | | | | | | | |
| IFI27, OAS1, IFI44L, SIGLEC1, | 0.870 | 0.861 | 0.880 | 0.919 | 0.865 | 0.974 | 0.943 | 0.868 | 1 |
| ISG15, HERC6, SLC1A2, FAM20A | | | | | | | | | |
| RSAD2, OAS1, SIGLEC1, ISG15, | 0.870 | 0.860 | 0.880 | 0.913 | 0.854 | 0.972 | 0.958 | 0.902 | 1 |
| HERC6, IL1R2, FAM20A, RETN | | | | | | | | | |
| IFIT1, SIGLEC1, ISG15, SLC1A2, | 0.870 | 0.861 | 0.880 | 0.921 | 0.869 | 0.973 | 0.952 | 0.895 | 1 |
| IL1R2, OLAH, FAM20A, RETN | | | | | | | | | |
| OAS1, IFIT1, IFI44L, SIGLEC1, | 0.870 | 0.861 | 0.880 | 0.921 | 0.870 | 0.973 | 0.930 | 0.854 | 1 |
| ISG15, HERC6, SLC1A2, FAM20A | | | | | | | | | |
| IFI27, OAS1, IFIT1, IFI44L, ISG15, | 0.870 | 0.860 | 0.881 | 0.911 | 0.851 | 0.971 | 0.918 | 0.837 | 1 |
| HERC6, FAM20A, MMP8 | | | | | | | | | |
| RSAD2, OAS1, IFIT1, IFI44L, | 0.870 | 0.861 | 0.880 | 0.909 | 0.847 | 0.971 | 0.932 | 0.860 | 1 |
| SIGLEC1, HERC6, FAM20A, MMP8 | | | | | | | | | |
| OAS1, IFIT1, IFI44L, SIGLEC1, | 0.870 | 0.860 | 0.880 | 0.912 | 0.852 | 0.972 | 0.939 | 0.872 | 1 |
| ISG15, HERC6, FAM20A, MMP8 | | | | | | | | | |
| RSAD2, OAS1, IFIT1, SIGLEC1, | 0.870 | 0.861 | 0.880 | 0.919 | 0.863 | 0.974 | 0.946 | 0.883 | 1 |
| ISG15, SLC1A2, OLAH, FAM20A | | | | | | | | | |
| IFI27, OAS1, IFIT1, SIGLEC1, | 0.870 | 0.862 | 0.879 | 0.924 | 0.879 | 0.970 | 0.951 | 0.902 | 1.000 |
| ISG15, SLC1A2, IL1R2, MMP8 | | | | | | | | | |
| RSAD2, IFIT1, SIGLEC1, SLC1A2, | 0.870 | 0.860 | 0.880 | 0.922 | 0.869 | 0.975 | 0.953 | 0.896 | 1 |
| OLAH, FAM20A, RETN, MMP8 | | | | | | | | | |
| RSAD2, IFI27, IFIT1, HERC6, | 0.870 | 0.861 | 0.879 | 0.923 | 0.873 | 0.973 | 0.943 | 0.885 | 1 |
| SLC1A2, IL1R2, RETN, MMP8 | | | | | | | | | |
| RSAD2, IFIT1, IFI44L, SIGLEC1, | 0.870 | 0.861 | 0.879 | 0.924 | 0.874 | 0.975 | 0.932 | 0.855 | 1 |
| HERC6, SLC1A2, FAM20A, RETN | | | | | | | | | |
| IFI27, OAS1, IFIT1, HERC6, | 0.870 | 0.860 | 0.880 | 0.924 | 0.867 | 0.980 | 0.932 | 0.848 | 1 |
| SLC1A2, FAM20A, RETN, MMP8 | | | | | | | | | |
| IFI44L, SIGLEC1, ISG15, SLC1A2, | 0.870 | 0.860 | 0.880 | 0.915 | 0.859 | 0.972 | 0.949 | 0.880 | 1 |
| OLAH, FAM20A, RETN, MMP8 | | | | | | | | | |
| RSAD2, IFI27, OAS1, HERC6, | 0.870 | 0.860 | 0.880 | 0.923 | 0.867 | 0.980 | 0.932 | 0.848 | 1 |
| SLC1A2, FAM20A, RETN, MMP8 | | | | | | | | | |
| IFI44L, SIGLEC1, ISG15, SLC1A2, | 0.870 | 0.860 | 0.879 | 0.917 | 0.864 | 0.969 | 0.954 | 0.891 | 1 |
| IL1R2, OLAH, FAM20A, RETN | | | | | | | | | |
| OAS1, SIGLEC1, ISG15, HERC6, | 0.870 | 0.859 | 0.880 | 0.920 | 0.869 | 0.971 | 0.946 | 0.878 | 1 |
| SLC1A2, IL1R2, OLAH, FAM20A | | | | | | | | | |
| IFI27, IFI44L, SIGLEC1, ISG15, | 0.870 | 0.862 | 0.878 | 0.926 | 0.881 | 0.970 | 0.943 | 0.892 | 0.995 |
| HERC6, SLC1A2, IL1R2, MMP8 | | | | | | | | | |
| SIGLEC1, ISG15, HERC6, SLC1A2, | 0.870 | 0.859 | 0.880 | 0.918 | 0.862 | 0.974 | 0.948 | 0.875 | 1 |
| OLAH, FAM20A, RETN, MMP8 | | | | | | | | | |
| IFI27, IFIT1, IFI44L, SIGLEC1, | 0.870 | 0.861 | 0.879 | 0.926 | 0.881 | 0.971 | 0.948 | 0.900 | 0.996 |
| HERC6, SLC1A2, IL1R2, MMP8 | | | | | | | | | |
| RSAD2, SIGLEC1, ISG15, HERC6, | 0.870 | 0.860 | 0.880 | 0.916 | 0.856 | 0.975 | 0.952 | 0.895 | 1 |
| OLAH, FAM20A, RETN, MMP8 | | | | | | | | | |
| RSAD2, OAS1, IFIT1, SIGLEC1, | 0.870 | 0.860 | 0.879 | 0.921 | 0.869 | 0.973 | 0.938 | 0.866 | 1 |
| HERC6, SLC1A2, FAM20A, RETN | | | | | | | | | |
| RSAD2, IFI27, IFI44L, SIGLEC1, | 0.870 | 0.861 | 0.878 | 0.926 | 0.880 | 0.972 | 0.948 | 0.900 | 0.996 |
| HERC6, SLC1A2, IL1R2, MMP8 | | | | | | | | | |
| IFI27, OAS1, IFIT1, IFI44L, ISG15, | 0.870 | 0.861 | 0.879 | 0.920 | 0.872 | 0.969 | 0.936 | 0.879 | 0.992 |
| HERC6, IL1R2, MMP8 | | | | | | | | | |
| OAS1, IFIT1, IFI44L, SIGLEC1, | 0.870 | 0.860 | 0.879 | 0.919 | 0.867 | 0.971 | 0.952 | 0.893 | 1 |
| SLC1A2, IL1R2, FAM20A, RETN | | | | | | | | | |
| RSAD2, IFIT1, IFI44L, SIGLEC1, | 0.870 | 0.860 | 0.879 | 0.921 | 0.870 | 0.973 | 0.933 | 0.856 | 1 |
| HERC6, SLC1A2, FAM20A, MMP8 | | | | | | | | | |
| RSAD2, IFI27, IFIT1, IFI44L, | 0.870 | 0.860 | 0.879 | 0.918 | 0.863 | 0.974 | 0.943 | 0.887 | 1.000 |
| ISG15, IL1R2, RETN, MMP8 | | | | | | | | | |
| RSAD2, IFI27, OAS1, IFIT1, | 0.870 | 0.861 | 0.878 | 0.925 | 0.880 | 0.971 | 0.954 | 0.905 | 1 |
| SIGLEC1, ISG15, SLC1A2, IL1R2 | | | | | | | | | |
| IFI27, OAS1, IFI44L, ISG15, | 0.870 | 0.860 | 0.879 | 0.915 | 0.857 | 0.972 | 0.933 | 0.859 | 1 |
| HERC6, SLC1A2, RETN, MMP8 | | | | | | | | | |
| RSAD2, IFI27, OAS1, IFIT1, | 0.870 | 0.861 | 0.879 | 0.908 | 0.845 | 0.971 | 0.953 | 0.891 | 1 |
| IFI44L, SIGLEC1, ISG15, RETN | | | | | | | | | |
| RSAD2, IFI27, OAS1, IFIT1, | 0.870 | 0.861 | 0.878 | 0.921 | 0.873 | 0.969 | 0.957 | 0.913 | 1 |
| SIGLEC1, ISG15, IL1R2, MMP8 | | | | | | | | | |
| RSAD2, OAS1, SIGLEC1, HERC6, | 0.870 | 0.860 | 0.880 | 0.912 | 0.853 | 0.972 | 0.958 | 0.902 | 1 |
| IL1R2, OLAH, FAM20A, RETN | | | | | | | | | |
| RSAD2, IFIT1, SIGLEC1, ISG15, | 0.870 | 0.860 | 0.879 | 0.919 | 0.866 | 0.972 | 0.939 | 0.865 | 1 |
| HERC6, SLC1A2, FAM20A, RETN | | | | | | | | | |
| RSAD2, IFI27, OAS1, IFIT1, | 0.870 | 0.860 | 0.879 | 0.915 | 0.855 | 0.975 | 0.929 | 0.852 | 1 |
| IFI44L, HERC6, SLC1A2, RETN | | | | | | | | | |
| IFI27, IFIT1, IFI44L, SIGLEC1, | 0.870 | 0.861 | 0.878 | 0.918 | 0.863 | 0.973 | 0.947 | 0.893 | 1 |
| ISG15, HERC6, SLC1A2, RETN | | | | | | | | | |
| OAS1, SIGLEC1, ISG15, HERC6, | 0.870 | 0.860 | 0.880 | 0.913 | 0.852 | 0.974 | 0.961 | 0.911 | 1 |
| OLAH, FAM20A, RETN, MMP8 | | | | | | | | | |
| RSAD2, IFI27, IFIT1, IFI44L, | 0.870 | 0.861 | 0.878 | 0.919 | 0.868 | 0.969 | 0.935 | 0.878 | 0.992 |
| ISG15, HERC6, IL1R2, MMP8 | | | | | | | | | |
| OAS1, IFIT1, SIGLEC1, ISG15, | 0.870 | 0.860 | 0.880 | 0.919 | 0.864 | 0.974 | 0.945 | 0.882 | 1 |
| SLC1A2, OLAH, FAM20A, RETN | | | | | | | | | |
| RSAD2, IFI27, IFIT1, SIGLEC1, | 0.870 | 0.861 | 0.878 | 0.922 | 0.869 | 0.974 | 0.939 | 0.881 | 0.997 |
| ISG15, HERC6, SLC1A2, RETN | | | | | | | | | |
| RSAD2, IFI27, IFI44L, SIGLEC1, | 0.870 | 0.862 | 0.878 | 0.925 | 0.879 | 0.971 | 0.950 | 0.903 | 0.997 |
| ISG15, SLC1A2, IL1R2, MMP8 | | | | | | | | | |
| IFI27, IFIT1, IFI44L, SIGLEC1, | 0.870 | 0.861 | 0.878 | 0.924 | 0.878 | 0.970 | 0.947 | 0.897 | 0.997 |
| ISG15, HERC6, SLC1A2, IL1R2 | | | | | | | | | |
| RSAD2, IFI27, OAS1, IFIT1, | 0.870 | 0.861 | 0.878 | 0.926 | 0.880 | 0.972 | 0.950 | 0.898 | 1 |
| IFI44L, SIGLEC1, SLC1A2, IL1R2 | | | | | | | | | |
| RSAD2, SIGLEC1, ISG15, HERC6, | 0.870 | 0.859 | 0.880 | 0.919 | 0.867 | 0.971 | 0.946 | 0.873 | 1 |
| SLC1A2, IL1R2, OLAH, FAM20A | | | | | | | | | |
| RSAD2, IFI27, OAS1, IFI44L, | 0.870 | 0.861 | 0.878 | 0.927 | 0.881 | 0.973 | 0.955 | 0.910 | 1 |
| SIGLEC1, ISG15, SLC1A2, IL1R2 | | | | | | | | | |
| IFI27, IFIT1, ISG15, HERC6, | 0.870 | 0.859 | 0.880 | 0.920 | 0.864 | 0.977 | 0.937 | 0.856 | 1 |
| SLC1A2, FAM20A, RETN, MMP8 | | | | | | | | | |
| RSAD2, OAS1, SIGLEC1, ISG15, | 0.869 | 0.859 | 0.880 | 0.919 | 0.868 | 0.969 | 0.951 | 0.881 | 1 |
| HERC6, SLC1A2, IL1R2, FAM20A | | | | | | | | | |
| RSAD2, IFI27, SIGLEC1, ISG15, | 0.869 | 0.861 | 0.878 | 0.923 | 0.878 | 0.969 | 0.948 | 0.898 | 0.997 |
| HERC6, SLC1A2, IL1R2, MMP8 | | | | | | | | | |
| RSAD2, IFI27, IFIT1, IFI44L, | 0.869 | 0.861 | 0.878 | 0.926 | 0.880 | 0.971 | 0.948 | 0.900 | 0.996 |
| SIGLEC1, HERC6, SLC1A2, IL1R2 | | | | | | | | | |
| RSAD2, IFIT1, IFI44L, SIGLEC1, | 0.869 | 0.860 | 0.878 | 0.920 | 0.868 | 0.973 | 0.955 | 0.897 | 1 |
| ISG15, SLC1A2, IL1R2, FAM20A | | | | | | | | | |
| RSAD2, IFI27, IFIT1, SIGLEC1, | 0.869 | 0.861 | 0.878 | 0.921 | 0.868 | 0.974 | 0.942 | 0.887 | 0.998 |
| HERC6, SLC1A2, RETN, MMP8 | | | | | | | | | |
| RSAD2, OAS1, SIGLEC1, HERC6, | 0.869 | 0.859 | 0.880 | 0.919 | 0.868 | 0.971 | 0.946 | 0.878 | 1 |
| SLC1A2, IL1R2, OLAH, FAM20A | | | | | | | | | |
| RSAD2, IFI27, IFIT1, SIGLEC1, | 0.869 | 0.861 | 0.878 | 0.925 | 0.880 | 0.970 | 0.949 | 0.900 | 0.998 |
| ISG15, HERC6, SLC1A2, IL1R2 | | | | | | | | | |
| IFI27, IFIT1, SIGLEC1, ISG15, | 0.869 | 0.861 | 0.878 | 0.925 | 0.880 | 0.970 | 0.950 | 0.902 | 0.998 |
| HERC6, SLC1A2, IL1R2, MMP8 | | | | | | | | | |
| OAS1, IFI44L, SIGLEC1, ISG15, | 0.869 | 0.859 | 0.879 | 0.920 | 0.869 | 0.970 | 0.949 | 0.878 | 1 |
| HERC6, SLC1A2, IL1R2, FAM20A | | | | | | | | | |
| IFI27, IFI44L, ISG15, HERC6, | 0.869 | 0.861 | 0.878 | 0.924 | 0.874 | 0.974 | 0.935 | 0.868 | 1 |
| SLC1A2, IL1R2, RETN, MMP8 | | | | | | | | | |
| RSAD2, IFI27, IFI44L, SIGLEC1, | 0.869 | 0.861 | 0.878 | 0.925 | 0.879 | 0.971 | 0.950 | 0.903 | 0.997 |
| ISG15, HERC6, SLC1A2, IL1R2 | | | | | | | | | |
| RSAD2, IFI27, IFIT1, IFI44L, | 0.869 | 0.860 | 0.879 | 0.915 | 0.856 | 0.973 | 0.938 | 0.879 | 0.997 |
| ISG15, HERC6, RETN, MMP8 | | | | | | | | | |
| RSAD2, IFI27, OAS1, IFI44L, | 0.869 | 0.861 | 0.878 | 0.926 | 0.879 | 0.972 | 0.952 | 0.902 | 1 |
| SIGLEC1, SLC1A2, IL1R2, MMP8 | | | | | | | | | |
| RSAD2, IFIT1, IFI44L, SIGLEC1, | 0.869 | 0.860 | 0.879 | 0.911 | 0.848 | 0.974 | 0.963 | 0.913 | 1 |
| ISG15, IL1R2, FAM20A, RETN | | | | | | | | | |
| RSAD2, OAS1, SIGLEC1, ISG15, | 0.869 | 0.859 | 0.880 | 0.910 | 0.849 | 0.971 | 0.953 | 0.898 | 1 |
| HERC6, OLAH, FAM20A, MMP8 | | | | | | | | | |
| RSAD2, OAS1, IFIT1, SIGLEC1, | 0.869 | 0.860 | 0.879 | 0.918 | 0.865 | 0.972 | 0.957 | 0.895 | 1 |
| ISG15, SLC1A2, IL1R2, FAM20A | | | | | | | | | |
| IFI27, IFI44L, SIGLEC1, ISG15, | 0.869 | 0.859 | 0.879 | 0.916 | 0.860 | 0.973 | 0.942 | 0.865 | 1 |
| HERC6, SLC1A2, FAM20A, MMP8 | | | | | | | | | |
| RSAD2, OAS1, IFIT1, SIGLEC1, | 0.869 | 0.860 | 0.879 | 0.918 | 0.864 | 0.971 | 0.937 | 0.860 | 1 |
| ISG15, HERC6, SLC1A2, FAM20A | | | | | | | | | |
| IFI27, IFIT1, IFI44L, HERC6, | 0.869 | 0.860 | 0.878 | 0.923 | 0.872 | 0.974 | 0.940 | 0.879 | 1 |
| SLC1A2, IL1R2, RETN, MMP8 | | | | | | | | | |
| RSAD2, OAS1, IFIT1, SIGLEC1, | 0.869 | 0.859 | 0.879 | 0.919 | 0.863 | 0.975 | 0.951 | 0.893 | 1 |
| SLC1A2, OLAH, FAM20A, RETN | | | | | | | | | |
| RSAD2, IFI27, ISG15, HERC6, | 0.869 | 0.860 | 0.878 | 0.925 | 0.876 | 0.974 | 0.936 | 0.870 | 1 |
| SLC1A2, IL1R2, RETN, MMP8 | | | | | | | | | |
| RSAD2, OAS1, IFIT1, IFI44L, | 0.869 | 0.860 | 0.879 | 0.920 | 0.868 | 0.973 | 0.952 | 0.892 | 1 |
| SIGLEC1, SLC1A2, IL1R2, FAM20A | | | | | | | | | |
| OAS1, SIGLEC1, ISG15, SLC1A2, | 0.869 | 0.859 | 0.879 | 0.915 | 0.858 | 0.971 | 0.950 | 0.888 | 1 |
| OLAH, FAM20A, RETN, MMP8 | | | | | | | | | |
| RSAD2, IFI27, OAS1, ISG15, | 0.869 | 0.859 | 0.879 | 0.917 | 0.859 | 0.976 | 0.927 | 0.850 | 1 |
| HERC6, SLC1A2, RETN, MMP8 | | | | | | | | | |
| RSAD2, SIGLEC1, ISG15, HERC6, | 0.869 | 0.859 | 0.879 | 0.913 | 0.853 | 0.972 | 0.957 | 0.899 | 1 |
| IL1R2, OLAH, FAM20A, RETN | | | | | | | | | |
| RSAD2, IFI27, IFI44L, SIGLEC1, | 0.869 | 0.860 | 0.878 | 0.921 | 0.867 | 0.975 | 0.941 | 0.883 | 1.000 |
| ISG15, HERC6, SLC1A2, RETN | | | | | | | | | |
| IFI27, OAS1, IFI44L, SIGLEC1, | 0.869 | 0.861 | 0.877 | 0.924 | 0.878 | 0.970 | 0.945 | 0.893 | 0.996 |
| ISG15, SLC1A2, IL1R2, MMP8 | | | | | | | | | |
| RSAD2, IFI27, IFI44L, HERC6, | 0.869 | 0.859 | 0.880 | 0.924 | 0.868 | 0.980 | 0.937 | 0.857 | 1 |
| SLC1A2, FAM20A, RETN, MMP8 | | | | | | | | | |
| RSAD2, OAS1, SIGLEC1, SLC1A2, | 0.869 | 0.859 | 0.879 | 0.918 | 0.862 | 0.973 | 0.951 | 0.891 | 1 |
| OLAH, FAM20A, RETN, MMP8 | | | | | | | | | |
| RSAD2, OAS1, IFIT1, IFI44L, | 0.869 | 0.859 | 0.879 | 0.905 | 0.835 | 0.974 | 0.948 | 0.893 | 1 |
| SIGLEC1, OLAH, FAM20A, RETN | | | | | | | | | |
| RSAD2, IFI27, IFIT1, HERC6, | 0.869 | 0.859 | 0.879 | 0.923 | 0.867 | 0.980 | 0.934 | 0.852 | 1 |
| SLC1A2, FAM20A, RETN, MMP8 | | | | | | | | | |
| OAS1, IFIT1, SIGLEC1, ISG15, | 0.869 | 0.859 | 0.879 | 0.922 | 0.871 | 0.972 | 0.951 | 0.883 | 1 |
| SLC1A2, IL1R2, FAM20A, RETN | | | | | | | | | |
| IFI27, IFIT1, IFI44L, SIGLEC1, | 0.869 | 0.860 | 0.878 | 0.920 | 0.866 | 0.974 | 0.942 | 0.886 | 0.999 |
| HERC6, SLC1A2, RETN, MMP8 | | | | | | | | | |
| RSAD2, OAS1, IFIT1, SIGLEC1, | 0.869 | 0.859 | 0.878 | 0.916 | 0.861 | 0.971 | 0.937 | 0.860 | 1 |
| HERC6, SLC1A2, FAM20A, MMP8 | | | | | | | | | |
| RSAD2, IFI27, OAS1, IFIT1, | 0.869 | 0.858 | 0.879 | 0.911 | 0.849 | 0.973 | 0.921 | 0.838 | 1 |
| IFI44L, ISG15, HERC6, FAM20A | | | | | | | | | |
| RSAD2, OAS1, IFI44L, SIGLEC1, | 0.869 | 0.859 | 0.879 | 0.910 | 0.846 | 0.973 | 0.963 | 0.912 | 1 |
| ISG15, IL1R2, OLAH, FAM20A | | | | | | | | | |
| RSAD2, IFIT1, SIGLEC1, ISG15, | 0.869 | 0.859 | 0.878 | 0.922 | 0.871 | 0.973 | 0.955 | 0.891 | 1 |
| SLC1A2, IL1R2, FAM20A, RETN | | | | | | | | | |
| RSAD2, IFI27, OAS1, IFIT1, | 0.869 | 0.859 | 0.879 | 0.912 | 0.853 | 0.972 | 0.920 | 0.837 | 1 |
| IFI44L, HERC6, FAM20A, MMP8 | | | | | | | | | |
| RSAD2, IFI27, OAS1, IFI44L, | 0.869 | 0.858 | 0.879 | 0.916 | 0.858 | 0.975 | 0.946 | 0.872 | 1 |
| SIGLEC1, ISG15, SLC1A2, FAM20A | | | | | | | | | |
| IFI27, OAS1, IFIT1, IFI44L, | 0.869 | 0.859 | 0.878 | 0.914 | 0.855 | 0.972 | 0.932 | 0.859 | 1 |
| HERC6, SLC1A2, RETN, MMP8 | | | | | | | | | |
| RSAD2, IFIT1, SIGLEC1, ISG15, | 0.869 | 0.859 | 0.878 | 0.912 | 0.847 | 0.976 | 0.959 | 0.911 | 1 |
| OLAH, FAM20A, RETN, MMP8 | | | | | | | | | |
| RSAD2, IFIT1, IFI44L, SIGLEC1, | 0.869 | 0.859 | 0.879 | 0.915 | 0.853 | 0.978 | 0.959 | 0.911 | 1 |
| OLAH, FAM20A, RETN, MMP8 | | | | | | | | | |
| RSAD2, IFI27, OAS1, IFIT1, | 0.869 | 0.860 | 0.877 | 0.924 | 0.877 | 0.971 | 0.953 | 0.903 | 1 |
| SIGLEC1, SLC1A2, IL1R2, MMP8 | | | | | | | | | |
| OAS1, IFI44L, SIGLEC1, ISG15, | 0.869 | 0.859 | 0.879 | 0.914 | 0.855 | 0.973 | 0.961 | 0.908 | 1 |
| HERC6, IL1R2, FAM20A, RETN | | | | | | | | | |
| RSAD2, IFI44L, SIGLEC1, HERC6, | 0.869 | 0.858 | 0.879 | 0.918 | 0.866 | 0.971 | 0.945 | 0.871 | 1 |
| SLC1A2, IL1R2, OLAH, FAM20A | | | | | | | | | |
| RSAD2, OAS1, IFIT1, SIGLEC1, | 0.869 | 0.859 | 0.878 | 0.904 | 0.834 | 0.974 | 0.952 | 0.901 | 1 |
| ISG15, OLAH, FAM20A, RETN | | | | | | | | | |
| RSAD2, OAS1, SIGLEC1, ISG15, | 0.869 | 0.859 | 0.879 | 0.906 | 0.842 | 0.971 | 0.961 | 0.904 | 1 |
| IL1R2, OLAH, FAM20A, RETN | | | | | | | | | |
| RSAD2, IFI27, OAS1, SIGLEC1, | 0.868 | 0.860 | 0.877 | 0.926 | 0.880 | 0.971 | 0.954 | 0.905 | 1 |
| ISG15, SLC1A2, IL1R2, MMP8 | | | | | | | | | |
| RSAD2, IFIT1, IFI44L, SIGLEC1, | 0.868 | 0.859 | 0.878 | 0.920 | 0.869 | 0.972 | 0.954 | 0.895 | 1 |
| SLC1A2, IL1R2, FAM20A, RETN | | | | | | | | | |
| RSAD2, IFIT1, SIGLEC1, ISG15, | 0.868 | 0.859 | 0.878 | 0.919 | 0.864 | 0.974 | 0.947 | 0.885 | 1 |
| SLC1A2, OLAH, FAM20A, RETN | | | | | | | | | |
| RSAD2, IFI27, IFIT1, IFI44L, | 0.868 | 0.860 | 0.877 | 0.925 | 0.879 | 0.971 | 0.950 | 0.903 | 0.997 |
| SIGLEC1, SLC1A2, IL1R2, MMP8 | | | | | | | | | |
| IFI44L, SIGLEC1, HERC6, | 0.868 | 0.858 | 0.879 | 0.917 | 0.860 | 0.974 | 0.951 | 0.879 | 1 |
| SLC1A2, OLAH, FAM20A, RETN, MMP8 | | | | | | | | | |
| RSAD2, IFI27, IFIT1, SIGLEC1, | 0.868 | 0.860 | 0.877 | 0.924 | 0.878 | 0.969 | 0.950 | 0.901 | 0.999 |
| ISG15, SLC1A2, IL1R2, MMP8 | | | | | | | | | |
| RSAD2, OAS1, IFI44L, SIGLEC1, | 0.868 | 0.859 | 0.878 | 0.913 | 0.854 | 0.973 | 0.959 | 0.904 | 1 |
| HERC6, IL1R2, FAM20A, RETN | | | | | | | | | |
| RSAD2, IFI27, IFIT1, IFI44L, | 0.868 | 0.860 | 0.877 | 0.923 | 0.872 | 0.973 | 0.933 | 0.867 | 0.998 |
| HERC6, SLC1A2, IL1R2, RETN | | | | | | | | | |
| RSAD2, IFI27, IFIT1, ISG15, | 0.868 | 0.860 | 0.877 | 0.922 | 0.871 | 0.972 | 0.936 | 0.872 | 1.000 |
| HERC6, SLC1A2, IL1R2, RETN | | | | | | | | | |
| IFI44L, SIGLEC1, ISG15, HERC6, | 0.868 | 0.858 | 0.879 | 0.911 | 0.846 | 0.975 | 0.961 | 0.907 | 1 |
| OLAH, FAM20A, RETN, MMP8 | | | | | | | | | |
| OAS1, SIGLEC1, ISG15, HERC6, | 0.868 | 0.858 | 0.879 | 0.911 | 0.851 | 0.971 | 0.960 | 0.907 | 1 |
| IL1R2, OLAH, FAM20A, RETN | | | | | | | | | |
| RSAD2, IFI27, OAS1, IFI44L, | 0.868 | 0.859 | 0.878 | 0.914 | 0.853 | 0.974 | 0.930 | 0.854 | 1 |
| HERC6, SLC1A2, RETN, MMP8 | | | | | | | | | |
| RSAD2, IFI44L, SIGLEC1, ISG15, | 0.868 | 0.858 | 0.878 | 0.918 | 0.866 | 0.970 | 0.948 | 0.875 | 1 |
| HERC6, SLC1A2, IL1R2, FAM20A | | | | | | | | | |
| IFI27, IFI44L, ISG15, HERC6, | 0.868 | 0.857 | 0.879 | 0.919 | 0.861 | 0.978 | 0.942 | 0.863 | 1 |
| SLC1A2, FAM20A, RETN, MMP8 | | | | | | | | | |
| RSAD2, OAS1, IFIT1, SIGLEC1, | 0.868 | 0.859 | 0.878 | 0.920 | 0.869 | 0.972 | 0.957 | 0.896 | 1 |
| SLC1A2, IL1R2, FAM20A, RETN | | | | | | | | | |
| OAS1, IFI44L, SIGLEC1, HERC6, | 0.868 | 0.858 | 0.879 | 0.920 | 0.869 | 0.971 | 0.946 | 0.878 | 1 |
| SLC1A2, IL1R2, OLAH, FAM20A | | | | | | | | | |
| RSAD2, IFI27, OAS1, IFIT1, | 0.868 | 0.859 | 0.877 | 0.923 | 0.877 | 0.969 | 0.937 | 0.873 | 1 |
| HERC6, SLC1A2, IL1R2, MMP8 | | | | | | | | | |
| IFI27, IFIT1, SIGLEC1, ISG15, | 0.868 | 0.859 | 0.877 | 0.919 | 0.865 | 0.974 | 0.940 | 0.880 | 1 |
| HERC6, SLC1A2, RETN, MMP8 | | | | | | | | | |
| RSAD2, IFI27, OAS1, SIGLEC1, | 0.868 | 0.857 | 0.879 | 0.916 | 0.858 | 0.975 | 0.948 | 0.877 | 1 |
| ISG15, SLC1A2, FAM20A, MMP8 | | | | | | | | | |
| RSAD2, SIGLEC1, ISG15, SLC1A2, | 0.868 | 0.858 | 0.878 | 0.916 | 0.861 | 0.972 | 0.948 | 0.885 | 1 |
| OLAH, FAM20A, RETN, MMP8 | | | | | | | | | |
| RSAD2, IFI27, OAS1, IFIT1, | 0.868 | 0.858 | 0.878 | 0.910 | 0.846 | 0.974 | 0.945 | 0.869 | 1 |
| IFI44L, ISG15, FAM20A, RETN | | | | | | | | | |
| RSAD2, IFI27, IFIT1, IFI44L, | 0.868 | 0.860 | 0.876 | 0.925 | 0.878 | 0.971 | 0.949 | 0.900 | 0.997 |
| SIGLEC1, ISG15, SLC1A2, IL1R2 | | | | | | | | | |
| OAS1, IFIT1, IFI44L, SIGLEC1, | 0.868 | 0.858 | 0.878 | 0.909 | 0.842 | 0.975 | 0.946 | 0.884 | 1 |
| ISG15, FAM20A, RETN, MMP8 | | | | | | | | | |
| OAS1, IFI44L, SIGLEC1, HERC6, | 0.868 | 0.858 | 0.878 | 0.914 | 0.854 | 0.973 | 0.961 | 0.908 | 1 |
| IL1R2, OLAH, FAM20A, RETN | | | | | | | | | |
| RSAD2, IFIT1, SIGLEC1, ISG15, | 0.868 | 0.858 | 0.878 | 0.917 | 0.863 | 0.971 | 0.937 | 0.860 | 1 |
| HERC6, SLC1A2, FAM20A, MMP8 | | | | | | | | | |
| RSAD2, IFI27, IFI44L, ISG15, | 0.868 | 0.860 | 0.876 | 0.923 | 0.873 | 0.973 | 0.933 | 0.866 | 0.999 |
| HERC6, SLC1A2, IL1R2, RETN | | | | | | | | | |
| RSAD2, IFI27, OAS1, IFIT1, | 0.868 | 0.859 | 0.877 | 0.921 | 0.869 | 0.972 | 0.941 | 0.876 | 1 |
| SLC1A2, IL1R2, RETN, MMP8 | | | | | | | | | |
| RSAD2, IFI27, IFI44L, SIGLEC1, | 0.868 | 0.857 | 0.878 | 0.918 | 0.860 | 0.976 | 0.947 | 0.874 | 1 |
| ISG15, SLC1A2, FAM20A, MMP8 | | | | | | | | | |
| RSAD2, IFI27, IFI44L, HERC6, | 0.868 | 0.859 | 0.877 | 0.923 | 0.874 | 0.973 | 0.935 | 0.868 | 1 |
| SLC1A2, IL1R2, RETN, MMP8 | | | | | | | | | |
| RSAD2, IFI27, OAS1, IFI44L, | 0.868 | 0.857 | 0.879 | 0.911 | 0.850 | 0.972 | 0.926 | 0.847 | 1 |
| ISG15, HERC6, FAM20A, MMP8 | | | | | | | | | |
| RSAD2, OAS1, IFI44L, SIGLEC1, | 0.868 | 0.858 | 0.878 | 0.910 | 0.850 | 0.970 | 0.952 | 0.895 | 1 |
| HERC6, OLAH, FAM20A, MMP8 | | | | | | | | | |
| RSAD2, SIGLEC1, HERC6, | 0.868 | 0.857 | 0.878 | 0.919 | 0.868 | 0.971 | 0.947 | 0.877 | 1 |
| SLC1A2, IL1R2, OLAH, FAM20A, RETN | | | | | | | | | |
| RSAD2, OAS1, IFI44L, SIGLEC1, | 0.868 | 0.858 | 0.878 | 0.920 | 0.870 | 0.970 | 0.952 | 0.884 | 1 |
| HERC6, SLC1A2, IL1R2, FAM20A | | | | | | | | | |
| RSAD2, SIGLEC1, ISG15, HERC6, | 0.868 | 0.858 | 0.878 | 0.913 | 0.855 | 0.971 | 0.950 | 0.880 | 1 |
| SLC1A2, FAM20A, RETN, MMP8 | | | | | | | | | |
| RSAD2, IFI27, OAS1, IFI44L, | 0.868 | 0.857 | 0.878 | 0.918 | 0.859 | 0.977 | 0.947 | 0.875 | 1 |
| SIGLEC1, SLC1A2, FAM20A, MMP8 | | | | | | | | | |
| RSAD2, OAS1, SIGLEC1, SLC1A2, | 0.868 | 0.858 | 0.878 | 0.917 | 0.863 | 0.970 | 0.958 | 0.900 | 1 |
| IL1R2, OLAH, FAM20A, RETN | | | | | | | | | |
| OAS1, IFIT1, SIGLEC1, ISG15, | 0.868 | 0.858 | 0.878 | 0.918 | 0.864 | 0.971 | 0.936 | 0.859 | 1 |
| HERC6, SLC1A2, FAM20A, MMP8 | | | | | | | | | |
| IFI27, OAS1, IFIT1, IFI44L, | 0.868 | 0.858 | 0.877 | 0.921 | 0.869 | 0.972 | 0.939 | 0.876 | 1 |
| SLC1A2, IL1R2, RETN, MMP8 | | | | | | | | | |
| IFI27, IFIT1, ISG15, HERC6, | 0.868 | 0.859 | 0.877 | 0.925 | 0.875 | 0.975 | 0.945 | 0.887 | 1 |
| SLC1A2, IL1R2, RETN, MMP8 | | | | | | | | | |
| OAS1, IFI44L, SIGLEC1, ISG15, | 0.868 | 0.858 | 0.878 | 0.906 | 0.842 | 0.970 | 0.962 | 0.907 | 1 |
| IL1R2, OLAH, FAM20A, RETN | | | | | | | | | |
| RSAD2, SIGLEC1, ISG15, SLC1A2, | 0.868 | 0.858 | 0.878 | 0.917 | 0.864 | 0.969 | 0.954 | 0.891 | 1 |
| IL1R2, OLAH, FAM20A, RETN | | | | | | | | | |
| RSAD2, IFI27, OAS1, IFI44L, | 0.868 | 0.858 | 0.877 | 0.918 | 0.864 | 0.973 | 0.942 | 0.883 | 1 |
| ISG15, IL1R2, RETN, MMP8 | | | | | | | | | |
| RSAD2, IFI27, OAS1, IFIT1, | 0.868 | 0.858 | 0.877 | 0.914 | 0.853 | 0.974 | 0.927 | 0.848 | 1 |
| HERC6, SLC1A2, RETN, MMP8 | | | | | | | | | |
| IFI27, IFIT1, IFI44L, ISG15, | 0.867 | 0.859 | 0.876 | 0.924 | 0.874 | 0.974 | 0.933 | 0.866 | 0.999 |
| HERC6, SLC1A2, IL1R2, RETN | | | | | | | | | |
| IFI27, OAS1, IFIT1, ISG15, | 0.867 | 0.858 | 0.877 | 0.921 | 0.870 | 0.972 | 0.945 | 0.884 | 1 |
| SLC1A2, IL1R2, RETN, MMP8 | | | | | | | | | |
| OAS1, IFI44L, SIGLEC1, SLC1A2, | 0.867 | 0.857 | 0.878 | 0.914 | 0.856 | 0.971 | 0.950 | 0.892 | 1 |
| OLAH, FAM20A, RETN, MMP8 | | | | | | | | | |
| RSAD2, IFI44L, SIGLEC1, HERC6, | 0.867 | 0.857 | 0.877 | 0.914 | 0.855 | 0.973 | 0.957 | 0.899 | 1 |
| IL1R2, OLAH, FAM20A, RETN | | | | | | | | | |
| RSAD2, IFI27, SIGLEC1, ISG15, | 0.867 | 0.858 | 0.877 | 0.921 | 0.867 | 0.975 | 0.948 | 0.894 | 1 |
| HERC6, SLC1A2, RETN, MMP8 | | | | | | | | | |
| OAS1, IFI44L, SIGLEC1, ISG15, | 0.867 | 0.857 | 0.878 | 0.916 | 0.861 | 0.971 | 0.941 | 0.872 | 1 |
| HERC6, SLC1A2, OLAH, FAM20A | | | | | | | | | |
| OAS1, SIGLEC1, ISG15, HERC6, | 0.867 | 0.857 | 0.878 | 0.920 | 0.869 | 0.970 | 0.950 | 0.881 | 1 |
| SLC1A2, IL1R2, FAM20A, RETN | | | | | | | | | |
| IFI27, OAS1, IFI44L, SIGLEC1, | 0.867 | 0.857 | 0.878 | 0.916 | 0.858 | 0.975 | 0.946 | 0.872 | 1 |
| ISG15, SLC1A2, FAM20A, MMP8 | | | | | | | | | |
| RSAD2, OAS1, SIGLEC1, ISG15, | 0.867 | 0.857 | 0.877 | 0.916 | 0.861 | 0.970 | 0.955 | 0.894 | 1 |
| SLC1A2, IL1R2, OLAH, FAM20A | | | | | | | | | |
| RSAD2, IFIT1, IFI44L, SIGLEC1, | 0.867 | 0.858 | 0.877 | 0.904 | 0.835 | 0.974 | 0.947 | 0.892 | 1 |
| ISG15, OLAH, FAM20A, RETN | | | | | | | | | |
| RSAD2, IFI27, OAS1, IFIT1, ISG15, | 0.867 | 0.858 | 0.876 | 0.922 | 0.875 | 0.968 | 0.939 | 0.877 | 1 |
| HERC6, SLC1A2, IL1R2 | | | | | | | | | |
| IFI27, IFIT1, IFI44L, ISG15, | 0.867 | 0.858 | 0.876 | 0.922 | 0.871 | 0.973 | 0.940 | 0.880 | 1 |
| SLC1A2, IL1R2, RETN, MMP8 | | | | | | | | | |
| RSAD2, OAS1, SIGLEC1, HERC6, | 0.867 | 0.857 | 0.877 | 0.918 | 0.868 | 0.969 | 0.952 | 0.884 | 1 |
| SLC1A2, IL1R2, FAM20A, RETN | | | | | | | | | |
| RSAD2, OAS1, SIGLEC1, ISG15, | 0.867 | 0.857 | 0.878 | 0.916 | 0.862 | 0.971 | 0.943 | 0.875 | 1 |
| HERC6, SLC1A2, OLAH, FAM20A | | | | | | | | | |
| IFI44L, SIGLEC1, HERC6, | 0.867 | 0.857 | 0.877 | 0.917 | 0.864 | 0.971 | 0.951 | 0.879 | 1 |
| SLC1A2, IL1R2, OLAH, FAM20A, RETN | | | | | | | | | |
| RSAD2, IFI44L, SIGLEC1, ISG15, | 0.867 | 0.857 | 0.877 | 0.914 | 0.855 | 0.973 | 0.954 | 0.893 | 1 |
| HERC6, IL1R2, FAM20A, RETN | | | | | | | | | |
| IFI27, OAS1, IFIT1, IFI44L, | 0.867 | 0.857 | 0.877 | 0.911 | 0.850 | 0.972 | 0.954 | 0.896 | 1 |
| SIGLEC1, ISG15, RETN, MMP8 | | | | | | | | | |
| IFI44L, SIGLEC1, ISG15, HERC6, | 0.867 | 0.857 | 0.877 | 0.920 | 0.868 | 0.971 | 0.951 | 0.879 | 1 |
| SLC1A2, IL1R2, FAM20A, RETN | | | | | | | | | |
| RSAD2, OAS1, IFI44L, SIGLEC1, | 0.867 | 0.857 | 0.877 | 0.904 | 0.839 | 0.970 | 0.961 | 0.906 | 1 |
| IL1R2, OLAH, FAM20A, RETN | | | | | | | | | |
| OAS1, SIGLEC1, HERC6, SLC1A2, | 0.867 | 0.856 | 0.878 | 0.921 | 0.871 | 0.971 | 0.949 | 0.882 | 1 |
| IL1R2, OLAH, FAM20A, RETN | | | | | | | | | |
| RSAD2, IFI27, OAS1, IFI44L, | 0.867 | 0.858 | 0.876 | 0.923 | 0.877 | 0.969 | 0.928 | 0.857 | 1.000 |
| HERC6, SLC1A2, IL1R2, MMP8 | | | | | | | | | |
| IFI27, OAS1, IFIT1, ISG15, HERC6, | 0.867 | 0.858 | 0.876 | 0.922 | 0.876 | 0.968 | 0.939 | 0.877 | 1 |
| SLC1A2, IL1R2, MMP8 | | | | | | | | | |
| IFI27, OAS1, IFIT1, IFI44L, | 0.867 | 0.858 | 0.876 | 0.923 | 0.877 | 0.969 | 0.936 | 0.875 | 0.997 |
| HERC6, SLC1A2, IL1R2, MMP8 | | | | | | | | | |
| IFI27, OAS1, IFIT1, IFI44L, | 0.867 | 0.858 | 0.876 | 0.914 | 0.858 | 0.971 | 0.949 | 0.883 | 1 |
| SIGLEC1, ISG15, SLC1A2, RETN | | | | | | | | | |
| RSAD2, OAS1, SIGLEC1, HERC6, | 0.867 | 0.857 | 0.877 | 0.913 | 0.855 | 0.971 | 0.949 | 0.878 | 1 |
| SLC1A2, FAM20A, RETN, MMP8 | | | | | | | | | |
| OAS1, SIGLEC1, ISG15, SLC1A2, | 0.867 | 0.857 | 0.877 | 0.918 | 0.865 | 0.970 | 0.955 | 0.894 | 1 |
| IL1R2, OLAH, FAM20A, RETN | | | | | | | | | |
| OAS1, IFI44L, SIGLEC1, HERC6, | 0.867 | 0.857 | 0.877 | 0.913 | 0.852 | 0.975 | 0.958 | 0.904 | 1 |
| OLAH, FAM20A, RETN, MMP8 | | | | | | | | | |
| RSAD2, IFI27, OAS1, IFIT1, | 0.867 | 0.858 | 0.876 | 0.922 | 0.875 | 0.968 | 0.938 | 0.876 | 1.000 |
| IFI44L, HERC6, SLC1A2, IL1R2 | | | | | | | | | |
| RSAD2, IFI27, OAS1, IFIT1, | 0.867 | 0.858 | 0.876 | 0.916 | 0.864 | 0.968 | 0.939 | 0.885 | 0.994 |
| IFI44L, ISG15, IL1R2, MMP8 | | | | | | | | | |
| IFI27, OAS1, IFIT1, IFI44L, ISG15, | 0.867 | 0.858 | 0.876 | 0.919 | 0.867 | 0.972 | 0.941 | 0.879 | 1 |
| SLC1A2, IL1R2, RETN | | | | | | | | | |
| OAS1, SIGLEC1, ISG15, HERC6, | 0.867 | 0.856 | 0.877 | 0.914 | 0.856 | 0.972 | 0.950 | 0.883 | 1 |
| SLC1A2, FAM20A, RETN, MMP8 | | | | | | | | | |
| RSAD2, IFI27, OAS1, IFIT1, ISG15, | 0.867 | 0.858 | 0.875 | 0.921 | 0.870 | 0.972 | 0.934 | 0.861 | 1 |
| SLC1A2, IL1R2, RETN | | | | | | | | | |
| RSAD2, SIGLEC1, ISG15, HERC6, | 0.867 | 0.856 | 0.877 | 0.918 | 0.866 | 0.969 | 0.948 | 0.875 | 1 |
| SLC1A2, IL1R2, FAM20A, RETN | | | | | | | | | |
| RSAD2, IFI27, OAS1, IFIT1, | 0.867 | 0.858 | 0.875 | 0.915 | 0.857 | 0.973 | 0.948 | 0.879 | 1 |
| SIGLEC1, ISG15, SLC1A2, RETN | | | | | | | | | |
| IFI27, OAS1, IFIT1, SIGLEC1, | 0.867 | 0.857 | 0.876 | 0.916 | 0.860 | 0.972 | 0.949 | 0.883 | 1 |
| ISG15, SLC1A2, RETN, MMP8 | | | | | | | | | |
| OAS1, IFI44L, SIGLEC1, HERC6, | 0.867 | 0.856 | 0.877 | 0.920 | 0.870 | 0.970 | 0.950 | 0.881 | 1 |
| SLC1A2, IL1R2, FAM20A, RETN | | | | | | | | | |
| RSAD2, IFI27, IFI44L, SIGLEC1, | 0.866 | 0.858 | 0.875 | 0.922 | 0.868 | 0.975 | 0.941 | 0.883 | 0.999 |
| HERC6, SLC1A2, RETN, MMP8 | | | | | | | | | |
| RSAD2, IFI27, OAS1, IFIT1, | 0.866 | 0.858 | 0.875 | 0.919 | 0.867 | 0.972 | 0.930 | 0.856 | 1 |
| IFI44L, SLC1A2, IL1R2, RETN | | | | | | | | | |
| RSAD2, IFI27, IFIT1, ISG15, | 0.866 | 0.858 | 0.875 | 0.922 | 0.871 | 0.972 | 0.946 | 0.887 | 1 |
| SLC1A2, IL1R2, RETN, MMP8 | | | | | | | | | |
| RSAD2, IFI44L, SIGLEC1, | 0.866 | 0.856 | 0.877 | 0.915 | 0.858 | 0.972 | 0.949 | 0.888 | 1 |
| SLC1A2, OLAH, FAM20A, RETN, MMP8 | | | | | | | | | |
| RSAD2, IFI44L, SIGLEC1, ISG15, | 0.866 | 0.857 | 0.876 | 0.913 | 0.852 | 0.974 | 0.950 | 0.885 | 1 |
| HERC6, FAM20A, RETN, MMP8 | | | | | | | | | |
| RSAD2, IFI44L, SIGLEC1, ISG15, | 0.866 | 0.856 | 0.877 | 0.917 | 0.862 | 0.972 | 0.938 | 0.867 | 1 |
| HERC6, SLC1A2, OLAH, FAM20A | | | | | | | | | |
| OAS1, IFIT1, IFI44L, SIGLEC1, | 0.866 | 0.856 | 0.876 | 0.918 | 0.862 | 0.974 | 0.939 | 0.868 | 1 |
| SLC1A2, FAM20A, RETN, MMP8 | | | | | | | | | |
| OAS1, IFI44L, SIGLEC1, ISG15, | 0.866 | 0.856 | 0.876 | 0.917 | 0.864 | 0.971 | 0.954 | 0.893 | 1 |
| SLC1A2, IL1R2, OLAH, FAM20A | | | | | | | | | |
| RSAD2, IFI27, OAS1, IFIT1, | 0.866 | 0.858 | 0.875 | 0.916 | 0.859 | 0.974 | 0.948 | 0.878 | 1 |
| IFI44L, SIGLEC1, SLC1A2, RETN | | | | | | | | | |
| RSAD2, OAS1, IFIT1, SIGLEC1, | 0.866 | 0.856 | 0.876 | 0.909 | 0.843 | 0.974 | 0.952 | 0.889 | 1 |
| ISG15, FAM20A, RETN, MMP8 | | | | | | | | | |
| RSAD2, IFI44L, SIGLEC1, ISG15, | 0.866 | 0.856 | 0.876 | 0.906 | 0.843 | 0.970 | 0.962 | 0.909 | 1 |
| IL1R2, OLAH, FAM20A, RETN | | | | | | | | | |
| RSAD2, OAS1, SIGLEC1, ISG15, | 0.866 | 0.856 | 0.876 | 0.907 | 0.842 | 0.973 | 0.965 | 0.922 | 1 |
| OLAH, FAM20A, RETN, MMP8 | | | | | | | | | |
| SIGLEC1, ISG15, HERC6, SLC1A2, | 0.866 | 0.856 | 0.876 | 0.918 | 0.865 | 0.971 | 0.948 | 0.875 | 1 |
| IL1R2, OLAH, FAM20A, RETN | | | | | | | | | |
| RSAD2, IFI27, OAS1, ISG15, | 0.866 | 0.857 | 0.875 | 0.923 | 0.877 | 0.970 | 0.930 | 0.863 | 0.998 |
| HERC6, SLC1A2, IL1R2, MMP8 | | | | | | | | | |
| RSAD2, IFI44L, SIGLEC1, | 0.866 | 0.856 | 0.876 | 0.917 | 0.863 | 0.971 | 0.954 | 0.893 | 1 |
| SLC1A2, IL1R2, OLAH, FAM20A, RETN | | | | | | | | | |
| RSAD2, IFI44L, SIGLEC1, HERC6, | 0.866 | 0.856 | 0.876 | 0.915 | 0.855 | 0.975 | 0.952 | 0.895 | 1 |
| OLAH, FAM20A, RETN, MMP8 | | | | | | | | | |
| RSAD2, IFI27, IFIT1, IFI44L, | 0.866 | 0.857 | 0.875 | 0.921 | 0.870 | 0.973 | 0.941 | 0.881 | 1 |
| SLC1A2, IL1R2, RETN, MMP8 | | | | | | | | | |
| RSAD2, IFI27, OAS1, IFI44L, | 0.866 | 0.857 | 0.875 | 0.924 | 0.875 | 0.974 | 0.935 | 0.865 | 1 |
| SLC1A2, IL1R2, RETN, MMP8 | | | | | | | | | |
| RSAD2, IFI27, OAS1, IFIT1, | 0.866 | 0.856 | 0.876 | 0.910 | 0.848 | 0.972 | 0.952 | 0.889 | 1 |
| IFI44L, SIGLEC1, RETN, MMP8 | | | | | | | | | |
| RSAD2, OAS1, IFIT1, IFI44L, | 0.866 | 0.856 | 0.876 | 0.911 | 0.845 | 0.976 | 0.941 | 0.874 | 1 |
| SIGLEC1, FAM20A, RETN, MMP8 | | | | | | | | | |
| RSAD2, OAS1, IFI44L, SIGLEC1, | 0.866 | 0.855 | 0.876 | 0.918 | 0.863 | 0.972 | 0.942 | 0.873 | 1 |
| HERC6, SLC1A2, OLAH, FAM20A | | | | | | | | | |
| RSAD2, IFI44L, SIGLEC1, ISG15, | 0.866 | 0.856 | 0.876 | 0.916 | 0.862 | 0.971 | 0.954 | 0.893 | 1 |
| SLC1A2, IL1R2, OLAH, FAM20A | | | | | | | | | |
| OAS1, IFI44L, SIGLEC1, SLC1A2, | 0.866 | 0.856 | 0.876 | 0.917 | 0.863 | 0.970 | 0.958 | 0.900 | 1 |
| IL1R2, OLAH, FAM20A, RETN | | | | | | | | | |
| RSAD2, IFI27, OAS1, IFI44L, | 0.866 | 0.857 | 0.874 | 0.916 | 0.858 | 0.974 | 0.948 | 0.876 | 1 |
| SIGLEC1, ISG15, SLC1A2, RETN | | | | | | | | | |
| RSAD2, IFI44L, SIGLEC1, ISG15, | 0.866 | 0.855 | 0.876 | 0.910 | 0.849 | 0.970 | 0.953 | 0.898 | 1 |
| HERC6, OLAH, FAM20A, MMP8 | | | | | | | | | |
| IFIT1, IFI44L, SIGLEC1, ISG15, | 0.866 | 0.856 | 0.875 | 0.917 | 0.861 | 0.974 | 0.938 | 0.867 | 1 |
| SLC1A2, FAM20A, RETN, MMP8 | | | | | | | | | |
| RSAD2, OAS1, IFIT1, IFI44L, | 0.866 | 0.856 | 0.875 | 0.904 | 0.835 | 0.973 | 0.942 | 0.881 | 1 |
| SIGLEC1, ISG15, FAM20A, RETN | | | | | | | | | |
| IFI27, OAS1, IFIT1, IFI44L, ISG15, | 0.866 | 0.856 | 0.875 | 0.923 | 0.877 | 0.969 | 0.936 | 0.875 | 0.997 |
| HERC6, SLC1A2, IL1R2 | | | | | | | | | |
| OAS1, IFI44L, SIGLEC1, ISG15, | 0.865 | 0.856 | 0.875 | 0.915 | 0.861 | 0.968 | 0.953 | 0.885 | 1 |
| SLC1A2, IL1R2, FAM20A, RETN | | | | | | | | | |
| RSAD2, OAS1, IFIT1, IFI44L, | 0.865 | 0.855 | 0.876 | 0.904 | 0.834 | 0.973 | 0.939 | 0.870 | 1 |
| SIGLEC1, ISG15, FAM20A, MMP8 | | | | | | | | | |
| RSAD2, OAS1, IFI44L, SIGLEC1, | 0.865 | 0.855 | 0.876 | 0.907 | 0.844 | 0.971 | 0.961 | 0.904 | 1 |
| ISG15, IL1R2, FAM20A, RETN | | | | | | | | | |
| RSAD2, IFI27, OAS1, IFI44L, | 0.865 | 0.854 | 0.877 | 0.914 | 0.851 | 0.976 | 0.946 | 0.872 | 1 |
| ISG15, FAM20A, RETN, MMP8 | | | | | | | | | |
| OAS1, IFI44L, SIGLEC1, ISG15, | 0.865 | 0.855 | 0.875 | 0.911 | 0.848 | 0.974 | 0.950 | 0.883 | 1 |
| HERC6, FAM20A, RETN, MMP8 | | | | | | | | | |
| RSAD2, IFI44L, SIGLEC1, ISG15, | 0.865 | 0.855 | 0.875 | 0.915 | 0.862 | 0.968 | 0.952 | 0.882 | 1 |
| SLC1A2, IL1R2, FAM20A, RETN | | | | | | | | | |
| RSAD2, OAS1, SIGLEC1, ISG15, | 0.865 | 0.855 | 0.875 | 0.915 | 0.861 | 0.968 | 0.954 | 0.888 | 1 |
| SLC1A2, IL1R2, FAM20A, RETN | | | | | | | | | |
| RSAD2, IFI44L, SIGLEC1, HERC6, | 0.865 | 0.855 | 0.876 | 0.919 | 0.868 | 0.970 | 0.947 | 0.874 | 1 |
| SLC1A2, IL1R2, FAM20A, RETN | | | | | | | | | |
| IFI27, IFI44L, SIGLEC1, ISG15, | 0.865 | 0.856 | 0.875 | 0.917 | 0.861 | 0.973 | 0.941 | 0.882 | 1 |
| HERC6, SLC1A2, RETN, MMP8 | | | | | | | | | |
| IFI44L, ISG15, HERC6, SLC1A2, | 0.865 | 0.856 | 0.875 | 0.917 | 0.866 | 0.969 | 0.929 | 0.848 | 1 |
| IL1R2, OLAH, FAM20A, MMP8 | | | | | | | | | |
| IFIT1, IFI44L, ISG15, HERC6, | 0.865 | 0.855 | 0.875 | 0.917 | 0.863 | 0.971 | 0.922 | 0.840 | 1 |
| SLC1A2, OLAH, FAM20A, MMP8 | | | | | | | | | |
| RSAD2, IFI27, OAS1, IFIT1, ISG15, | 0.865 | 0.855 | 0.875 | 0.913 | 0.856 | 0.970 | 0.925 | 0.844 | 1 |
| HERC6, FAM20A, MMP8 | | | | | | | | | |
| RSAD2, OAS1, IFI44L, SIGLEC1, | 0.865 | 0.855 | 0.876 | 0.905 | 0.840 | 0.971 | 0.960 | 0.910 | 1 |
| ISG15, OLAH, FAM20A, MMP8 | | | | | | | | | |
| IFI27, OAS1, IFIT1, IFI44L, | 0.865 | 0.856 | 0.874 | 0.915 | 0.858 | 0.972 | 0.950 | 0.885 | 1 |
| SIGLEC1, SLC1A2, RETN, MMP8 | | | | | | | | | |
| OAS1, SIGLEC1, ISG15, HERC6, | 0.865 | 0.854 | 0.876 | 0.915 | 0.859 | 0.970 | 0.943 | 0.874 | 1 |
| SLC1A2, OLAH, FAM20A, RETN | | | | | | | | | |
| OAS1, IFIT1, IFI44L, SIGLEC1, | 0.865 | 0.855 | 0.875 | 0.912 | 0.852 | 0.971 | 0.938 | 0.864 | 1 |
| ISG15, SLC1A2, FAM20A, MMP8 | | | | | | | | | |
| RSAD2, OAS1, IFI44L, SIGLEC1, | 0.865 | 0.855 | 0.875 | 0.916 | 0.861 | 0.970 | 0.955 | 0.896 | 1 |
| SLC1A2, IL1R2, OLAH, FAM20A | | | | | | | | | |
| IFIT1, IFI44L, HERC6, SLC1A2, | 0.865 | 0.856 | 0.875 | 0.919 | 0.869 | 0.970 | 0.929 | 0.847 | 1 |
| IL1R2, OLAH, FAM20A, MMP8 | | | | | | | | | |
| RSAD2, OAS1, IFI44L, HERC6, | 0.865 | 0.855 | 0.875 | 0.912 | 0.854 | 0.970 | 0.936 | 0.860 | 1 |
| IL1R2, OLAH, FAM20A, MMP8 | | | | | | | | | |
| RSAD2, IFI27, OAS1, IFIT1, | 0.865 | 0.854 | 0.876 | 0.915 | 0.853 | 0.977 | 0.947 | 0.873 | 1 |
| IFI44L, FAM20A, RETN, MMP8 | | | | | | | | | |
| RSAD2, OAS1, ISG15, HERC6, | 0.865 | 0.855 | 0.875 | 0.909 | 0.851 | 0.967 | 0.937 | 0.859 | 1 |
| IL1R2, OLAH, FAM20A, MMP8 | | | | | | | | | |
| RSAD2, IFI27, OAS1, IFIT1, | 0.865 | 0.856 | 0.874 | 0.915 | 0.857 | 0.973 | 0.950 | 0.883 | 1 |
| SIGLEC1, SLC1A2, RETN, MMP8 | | | | | | | | | |
| RSAD2, IFI27, OAS1, ISG15, | 0.865 | 0.856 | 0.874 | 0.923 | 0.872 | 0.973 | 0.934 | 0.863 | 1 |
| SLC1A2, IL1R2, RETN, MMP8 | | | | | | | | | |
| RSAD2, IFI27, IFIT1, IFI44L, | 0.865 | 0.856 | 0.874 | 0.920 | 0.868 | 0.972 | 0.934 | 0.868 | 0.999 |
| ISG15, SLC1A2, IL1R2, RETN | | | | | | | | | |
| RSAD2, IFI44L, HERC6, SLC1A2, | 0.865 | 0.855 | 0.875 | 0.919 | 0.868 | 0.970 | 0.928 | 0.846 | 1 |
| IL1R2, OLAH, FAM20A, MMP8 | | | | | | | | | |
| RSAD2, IFI27, OAS1, IFI44L, | 0.865 | 0.856 | 0.874 | 0.924 | 0.878 | 0.969 | 0.933 | 0.867 | 0.998 |
| ISG15, HERC6, SLC1A2, IL1R2 | | | | | | | | | |
| RSAD2, IFI27, OAS1, IFIT1, | 0.865 | 0.856 | 0.873 | 0.921 | 0.874 | 0.969 | 0.940 | 0.879 | 1 |
| IFI44L, SLC1A2, IL1R2, MMP8 | | | | | | | | | |
| RSAD2, IFI27, IFIT1, IFI44L, | 0.865 | 0.854 | 0.876 | 0.914 | 0.851 | 0.977 | 0.942 | 0.867 | 1 |
| ISG15, FAM20A, RETN, MMP8 | | | | | | | | | |
| RSAD2, OAS1, IFIT1, HERC6, | 0.865 | 0.855 | 0.875 | 0.907 | 0.847 | 0.967 | 0.940 | 0.866 | 1 |
| IL1R2, OLAH, FAM20A, MMP8 | | | | | | | | | |
| OAS1, IFIT1, IFI44L, SIGLEC1, | 0.865 | 0.855 | 0.874 | 0.903 | 0.841 | 0.966 | 0.963 | 0.924 | 1 |
| HERC6, IL1R2, RETN, MMP8 | | | | | | | | | |
| RSAD2, OAS1, IFI44L, SIGLEC1, | 0.865 | 0.855 | 0.875 | 0.915 | 0.861 | 0.969 | 0.953 | 0.888 | 1 |
| SLC1A2, IL1R2, FAM20A, RETN | | | | | | | | | |
| RSAD2, OAS1, SIGLEC1, HERC6, | 0.865 | 0.854 | 0.875 | 0.915 | 0.860 | 0.970 | 0.943 | 0.875 | 1 |
| SLC1A2, OLAH, FAM20A, RETN | | | | | | | | | |
| RSAD2, OAS1, IFIT1, IFI44L, | 0.865 | 0.855 | 0.875 | 0.914 | 0.855 | 0.972 | 0.937 | 0.863 | 1 |
| SIGLEC1, SLC1A2, FAM20A, MMP8 | | | | | | | | | |
| IFI27, OAS1, IFIT1, IFI44L, ISG15, | 0.865 | 0.853 | 0.876 | 0.912 | 0.849 | 0.975 | 0.948 | 0.874 | 1 |
| FAM20A, RETN, MMP8 | | | | | | | | | |
| RSAD2, IFI27, OAS1, IFI44L, | 0.865 | 0.856 | 0.873 | 0.920 | 0.868 | 0.972 | 0.929 | 0.854 | 1 |
| ISG15, SLC1A2, IL1R2, RETN | | | | | | | | | |
| RSAD2, IFI27, OAS1, IFIT1, | 0.865 | 0.855 | 0.875 | 0.909 | 0.847 | 0.971 | 0.954 | 0.895 | 1 |
| SIGLEC1, ISG15, RETN, MMP8 | | | | | | | | | |
| RSAD2, IFIT1, IFI44L, SIGLEC1, | 0.865 | 0.855 | 0.874 | 0.912 | 0.853 | 0.970 | 0.937 | 0.863 | 1 |
| ISG15, SLC1A2, FAM20A, MMP8 | | | | | | | | | |
| RSAD2, OAS1, SIGLEC1, ISG15, | 0.865 | 0.855 | 0.875 | 0.908 | 0.846 | 0.970 | 0.951 | 0.896 | 1 |
| HERC6, OLAH, FAM20A, RETN | | | | | | | | | |
| RSAD2, SIGLEC1, ISG15, HERC6, | 0.865 | 0.854 | 0.875 | 0.915 | 0.860 | 0.970 | 0.941 | 0.871 | 1 |
| SLC1A2, OLAH, FAM20A, RETN | | | | | | | | | |
| RSAD2, IFI27, IFIT1, ISG15, | 0.865 | 0.856 | 0.873 | 0.923 | 0.878 | 0.968 | 0.936 | 0.876 | 0.996 |
| HERC6, SLC1A2, IL1R2, MMP8 | | | | | | | | | |
| OAS1, IFIT1, SIGLEC1, ISG15, | 0.865 | 0.855 | 0.874 | 0.908 | 0.849 | 0.968 | 0.963 | 0.925 | 1 |
| HERC6, IL1R2, RETN, MMP8 | | | | | | | | | |
| RSAD2, IFI27, OAS1, IFI44L, | 0.865 | 0.854 | 0.875 | 0.910 | 0.848 | 0.972 | 0.953 | 0.890 | 1 |
| SIGLEC1, ISG15, RETN, MMP8 | | | | | | | | | |
| RSAD2, IFI27, OAS1, IFIT1, | 0.865 | 0.856 | 0.873 | 0.922 | 0.875 | 0.969 | 0.938 | 0.874 | 1 |
| IFI44L, ISG15, SLC1A2, IL1R2 | | | | | | | | | |
| RSAD2, IFI27, OAS1, IFIT1, | 0.864 | 0.856 | 0.873 | 0.916 | 0.864 | 0.969 | 0.914 | 0.839 | 0.989 |
| SIGLEC1, HERC6, SLC1A2, MMP8 | | | | | | | | | |
| RSAD2, OAS1, IFI44L, SIGLEC1, | 0.864 | 0.854 | 0.874 | 0.915 | 0.861 | 0.969 | 0.954 | 0.891 | 1 |
| ISG15, SLC1A2, IL1R2, FAM20A | | | | | | | | | |
| RSAD2, OAS1, IFIT1, IFI44L, | 0.864 | 0.855 | 0.874 | 0.912 | 0.852 | 0.971 | 0.935 | 0.860 | 1 |
| SIGLEC1, ISG15, SLC1A2, FAM20A | | | | | | | | | |
| RSAD2, IFI27, IFIT1, IFI44L, | 0.864 | 0.856 | 0.873 | 0.924 | 0.878 | 0.969 | 0.935 | 0.875 | 0.995 |
| HERC6, SLC1A2, IL1R2, MMP8 | | | | | | | | | |
| RSAD2, IFIT1, IFI44L, HERC6, | 0.864 | 0.854 | 0.875 | 0.917 | 0.864 | 0.970 | 0.916 | 0.833 | 1.000 |
| SLC1A2, OLAH, FAM20A, MMP8 | | | | | | | | | |
| RSAD2, IFI27, OAS1, IFIT1, ISG15, | 0.864 | 0.856 | 0.873 | 0.921 | 0.873 | 0.968 | 0.936 | 0.871 | 1 |
| SLC1A2, IL1R2, MMP8 | | | | | | | | | |
| RSAD2, IFI27, IFI44L, ISG15, | 0.864 | 0.856 | 0.873 | 0.923 | 0.873 | 0.973 | 0.930 | 0.860 | 1 |
| SLC1A2, IL1R2, RETN, MMP8 | | | | | | | | | |
| IFI44L, ISG15, SLC1A2, IL1R2, | 0.864 | 0.855 | 0.874 | 0.917 | 0.864 | 0.970 | 0.929 | 0.851 | 1 |
| OLAH, FAM20A, RETN, MMP8 | | | | | | | | | |
| OAS1, IFIT1, SIGLEC1, ISG15, | 0.864 | 0.854 | 0.874 | 0.913 | 0.855 | 0.972 | 0.952 | 0.884 | 1 |
| SLC1A2, FAM20A, RETN, MMP8 | | | | | | | | | |
| IFIT1, IFI44L, ISG15, HERC6, | 0.864 | 0.855 | 0.874 | 0.909 | 0.851 | 0.968 | 0.937 | 0.860 | 1 |
| IL1R2, OLAH, FAM20A, MMP8 | | | | | | | | | |
| RSAD2, IFIT1, IFI44L, SIGLEC1, | 0.864 | 0.854 | 0.874 | 0.917 | 0.861 | 0.973 | 0.937 | 0.865 | 1 |
| SLC1A2, FAM20A, RETN, MMP8 | | | | | | | | | |
| OAS1, IFI44L, SIGLEC1, ISG15, | 0.864 | 0.854 | 0.875 | 0.908 | 0.845 | 0.970 | 0.959 | 0.907 | 1 |
| HERC6, OLAH, FAM20A, MMP8 | | | | | | | | | |
| IFIT1, IFI44L, HERC6, SLC1A2, | 0.864 | 0.854 | 0.874 | 0.919 | 0.865 | 0.974 | 0.924 | 0.842 | 1 |
| OLAH, FAM20A, RETN, MMP8 | | | | | | | | | |
| IFI44L, SIGLEC1, ISG15, HERC6, | 0.864 | 0.854 | 0.875 | 0.914 | 0.857 | 0.970 | 0.946 | 0.872 | 1 |
| SLC1A2, OLAH, FAM20A, RETN | | | | | | | | | |
| RSAD2, IFI27, IFIT1, IFI44L, | 0.864 | 0.856 | 0.873 | 0.923 | 0.878 | 0.969 | 0.935 | 0.875 | 0.995 |
| ISG15, HERC6, SLC1A2, IL1R2 | | | | | | | | | |
| RSAD2, OAS1, IFI44L, SIGLEC1, | 0.864 | 0.854 | 0.874 | 0.910 | 0.847 | 0.973 | 0.951 | 0.885 | 1 |
| HERC6, FAM20A, RETN, MMP8 | | | | | | | | | |
| RSAD2, OAS1, SIGLEC1, ISG15, | 0.864 | 0.854 | 0.874 | 0.915 | 0.857 | 0.972 | 0.949 | 0.888 | 1 |
| SLC1A2, OLAH, FAM20A, RETN | | | | | | | | | |
| RSAD2, OAS1, IFI44L, SIGLEC1, | 0.864 | 0.854 | 0.874 | 0.911 | 0.852 | 0.970 | 0.945 | 0.881 | 1 |
| ISG15, SLC1A2, OLAH, FAM20A | | | | | | | | | |
| IFIT1, IFI44L, ISG15, SLC1A2, | 0.864 | 0.855 | 0.873 | 0.916 | 0.863 | 0.968 | 0.928 | 0.848 | 1 |
| IL1R2, OLAH, FAM20A, MMP8 | | | | | | | | | |
| OAS1, IFI44L, SIGLEC1, ISG15, | 0.864 | 0.854 | 0.874 | 0.914 | 0.857 | 0.972 | 0.948 | 0.888 | 1 |
| SLC1A2, OLAH, FAM20A, RETN | | | | | | | | | |
| IFI27, OAS1, IFI44L, ISG15, | 0.864 | 0.855 | 0.873 | 0.921 | 0.875 | 0.967 | 0.928 | 0.861 | 0.995 |
| HERC6, SLC1A2, IL1R2, MMP8 | | | | | | | | | |
| RSAD2, OAS1, SIGLEC1, ISG15, | 0.864 | 0.853 | 0.874 | 0.912 | 0.855 | 0.969 | 0.941 | 0.869 | 1 |
| HERC6, SLC1A2, FAM20A, RETN | | | | | | | | | |
| OAS1, IFIT1, SIGLEC1, HERC6, | 0.864 | 0.854 | 0.874 | 0.915 | 0.860 | 0.970 | 0.954 | 0.910 | 0.999 |
| SLC1A2, OLAH, RETN, MMP8 | | | | | | | | | |
| RSAD2, OAS1, IFI44L, SIGLEC1, | 0.864 | 0.854 | 0.874 | 0.911 | 0.851 | 0.972 | 0.946 | 0.879 | 1 |
| ISG15, HERC6, FAM20A, RETN | | | | | | | | | |
| RSAD2, IFIT1, IFI44L, SIGLEC1, | 0.864 | 0.854 | 0.874 | 0.908 | 0.841 | 0.975 | 0.943 | 0.881 | 1 |
| ISG15, FAM20A, RETN, MMP8 | | | | | | | | | |
| IFI27, OAS1, IFIT1, IFI44L, | 0.864 | 0.853 | 0.874 | 0.917 | 0.863 | 0.971 | 0.910 | 0.820 | 1 |
| HERC6, SLC1A2, FAM20A, MMP8 | | | | | | | | | |
| OAS1, IFIT1, IFI44L, HERC6, | 0.864 | 0.854 | 0.873 | 0.908 | 0.849 | 0.967 | 0.938 | 0.862 | 1 |
| IL1R2, OLAH, FAM20A, MMP8 | | | | | | | | | |
| IFI27, OAS1, IFIT1, IFI44L, | 0.864 | 0.855 | 0.872 | 0.909 | 0.856 | 0.963 | 0.908 | 0.833 | 0.982 |
| SIGLEC1, ISG15, HERC6, MMP8 | | | | | | | | | |
| OAS1, IFI44L, SIGLEC1, HERC6, | 0.864 | 0.853 | 0.874 | 0.916 | 0.861 | 0.971 | 0.941 | 0.872 | 1 |
| SLC1A2, OLAH, FAM20A, RETN | | | | | | | | | |
| RSAD2, IFI27, IFIT1, IFI44L, | 0.864 | 0.853 | 0.874 | 0.916 | 0.861 | 0.971 | 0.914 | 0.826 | 1 |
| HERC6, SLC1A2, FAM20A, MMP8 | | | | | | | | | |
| IFIT1, IFI44L, ISG15, HERC6, | 0.864 | 0.854 | 0.874 | 0.917 | 0.865 | 0.969 | 0.921 | 0.834 | 1 |
| SLC1A2, IL1R2, OLAH, FAM20A | | | | | | | | | |
| OAS1, IFIT1, IFI44L, HERC6, | 0.864 | 0.853 | 0.874 | 0.918 | 0.865 | 0.970 | 0.916 | 0.830 | 1 |
| SLC1A2, OLAH, FAM20A, MMP8 | | | | | | | | | |
| RSAD2, OAS1, IFIT1, IFI44L, | 0.864 | 0.854 | 0.873 | 0.912 | 0.853 | 0.971 | 0.938 | 0.859 | 1 |
| HERC6, IL1R2, FAM20A, MMP8 | | | | | | | | | |
| RSAD2, OAS1, IFIT1, SIGLEC1, | 0.864 | 0.854 | 0.873 | 0.903 | 0.840 | 0.966 | 0.964 | 0.927 | 1 |
| HERC6, IL1R2, RETN, MMP8 | | | | | | | | | |
| IFI27, OAS1, IFI44L, ISG15, | 0.864 | 0.855 | 0.872 | 0.923 | 0.874 | 0.973 | 0.934 | 0.865 | 1 |
| SLC1A2, IL1R2, RETN, MMP8 | | | | | | | | | |
| IFI44L, HERC6, SLC1A2, IL1R2, | 0.864 | 0.854 | 0.873 | 0.917 | 0.864 | 0.971 | 0.933 | 0.852 | 1 |
| OLAH, FAM20A, RETN, MMP8 | | | | | | | | | |
| OAS1, IFIT1, IFI44L, ISG15, | 0.864 | 0.853 | 0.874 | 0.916 | 0.863 | 0.970 | 0.915 | 0.830 | 1 |
| HERC6, SLC1A2, OLAH, FAM20A | | | | | | | | | |
| RSAD2, OAS1, IFI44L, SIGLEC1, | 0.864 | 0.853 | 0.874 | 0.908 | 0.844 | 0.973 | 0.966 | 0.924 | 1 |
| OLAH, FAM20A, RETN, MMP8 | | | | | | | | | |
| RSAD2, IFI27, OAS1, IFIT1, ISG15, | 0.864 | 0.853 | 0.874 | 0.912 | 0.851 | 0.974 | 0.945 | 0.870 | 1 |
| FAM20A, RETN, MMP8 | | | | | | | | | |
| RSAD2, OAS1, IFI44L, SIGLEC1, | 0.864 | 0.853 | 0.874 | 0.908 | 0.846 | 0.970 | 0.950 | 0.893 | 1 |
| ISG15, HERC6, OLAH, FAM20A | | | | | | | | | |
| RSAD2, IFI27, OAS1, SIGLEC1, | 0.864 | 0.854 | 0.873 | 0.914 | 0.855 | 0.972 | 0.949 | 0.878 | 1 |
| ISG15, SLC1A2, RETN, MMP8 | | | | | | | | | |
| OAS1, IFI44L, SIGLEC1, ISG15, | 0.863 | 0.853 | 0.874 | 0.906 | 0.840 | 0.973 | 0.967 | 0.925 | 1 |
| OLAH, FAM20A, RETN, MMP8 | | | | | | | | | |
| RSAD2, IFI27, OAS1, IFIT1, | 0.863 | 0.855 | 0.872 | 0.907 | 0.849 | 0.964 | 0.903 | 0.825 | 0.982 |
| IFI44L, SIGLEC1, HERC6, MMP8 | | | | | | | | | |
| OAS1, IFIT1, IFI44L, SIGLEC1, | 0.863 | 0.854 | 0.873 | 0.914 | 0.855 | 0.973 | 0.936 | 0.863 | 1 |
| ISG15, SLC1A2, FAM20A, RETN | | | | | | | | | |
| OAS1, IFIT1, SIGLEC1, HERC6, | 0.863 | 0.853 | 0.873 | 0.904 | 0.841 | 0.967 | 0.964 | 0.927 | 1 |
| IL1R2, OLAH, RETN, MMP8 | | | | | | | | | |
| RSAD2, OAS1, HERC6, IL1R2, | 0.863 | 0.853 | 0.874 | 0.909 | 0.848 | 0.969 | 0.941 | 0.867 | 1 |
| OLAH, FAM20A, RETN, MMP8 | | | | | | | | | |
| IFI27, OAS1, IFI44L, SIGLEC1, | 0.863 | 0.854 | 0.873 | 0.915 | 0.858 | 0.972 | 0.950 | 0.885 | 1 |
| ISG15, SLC1A2, RETN, MMP8 | | | | | | | | | |
| OAS1, IFI44L, HERC6, SLC1A2, | 0.863 | 0.853 | 0.874 | 0.919 | 0.868 | 0.970 | 0.929 | 0.846 | 1 |
| IL1R2, OLAH, FAM20A, MMP8 | | | | | | | | | |
| IFI27, IFIT1, IFI44L, ISG15, | 0.863 | 0.855 | 0.872 | 0.923 | 0.877 | 0.969 | 0.934 | 0.873 | 0.994 |
| HERC6, SLC1A2, IL1R2, MMP8 | | | | | | | | | |
| RSAD2, IFI27, IFIT1, IFI44L, | 0.863 | 0.854 | 0.873 | 0.914 | 0.856 | 0.971 | 0.939 | 0.874 | 1 |
| ISG15, HERC6, SLC1A2, RETN | | | | | | | | | |
| RSAD2, IFI27, IFIT1, IFI44L, | 0.863 | 0.855 | 0.872 | 0.923 | 0.876 | 0.969 | 0.934 | 0.873 | 0.994 |
| ISG15, SLC1A2, IL1R2, MMP8 | | | | | | | | | |
| IFI44L, ISG15, HERC6, IL1R2, | 0.863 | 0.853 | 0.873 | 0.912 | 0.852 | 0.972 | 0.946 | 0.874 | 1 |
| OLAH, FAM20A, RETN, MMP8 | | | | | | | | | |
| IFI27, OAS1, IFIT1, IFI44L, ISG15, | 0.863 | 0.854 | 0.872 | 0.921 | 0.875 | 0.968 | 0.936 | 0.875 | 0.997 |
| SLC1A2, IL1R2, MMP8 | | | | | | | | | |
| RSAD2, OAS1, IFI44L, ISG15, | 0.863 | 0.853 | 0.873 | 0.910 | 0.851 | 0.968 | 0.936 | 0.863 | 1 |
| HERC6, IL1R2, OLAH, FAM20A | | | | | | | | | |
| IFIT1, IFI44L, SIGLEC1, ISG15, | 0.863 | 0.854 | 0.873 | 0.904 | 0.842 | 0.966 | 0.964 | 0.926 | 1 |
| HERC6, IL1R2, RETN, MMP8 | | | | | | | | | |
| RSAD2, IFI27, OAS1, IFI44L, | 0.863 | 0.854 | 0.873 | 0.915 | 0.857 | 0.974 | 0.950 | 0.879 | 1 |
| SIGLEC1, SLC1A2, RETN, MMP8 | | | | | | | | | |
| RSAD2, OAS1, ISG15, HERC6, | 0.863 | 0.853 | 0.873 | 0.910 | 0.851 | 0.969 | 0.935 | 0.853 | 1 |
| IL1R2, FAM20A, RETN, MMP8 | | | | | | | | | |
| IFI27, OAS1, IFIT1, IFI44L, ISG15, | 0.863 | 0.853 | 0.873 | 0.910 | 0.848 | 0.973 | 0.936 | 0.853 | 1 |
| SLC1A2, FAM20A, RETN | | | | | | | | | |
| RSAD2, IFI27, IFI44L, ISG15, | 0.863 | 0.854 | 0.872 | 0.920 | 0.874 | 0.967 | 0.926 | 0.859 | 0.993 |
| HERC6, SLC1A2, IL1R2, MMP8 | | | | | | | | | |
| OAS1, IFI44L, SIGLEC1, HERC6, | 0.863 | 0.853 | 0.873 | 0.912 | 0.854 | 0.971 | 0.950 | 0.881 | 1 |
| SLC1A2, FAM20A, RETN, MMP8 | | | | | | | | | |
| RSAD2, IFI44L, SIGLEC1, HERC6, | 0.863 | 0.852 | 0.874 | 0.917 | 0.862 | 0.972 | 0.939 | 0.868 | 1 |
| SLC1A2, OLAH, FAM20A, RETN | | | | | | | | | |
| RSAD2, IFI27, OAS1, IFIT1, | 0.863 | 0.854 | 0.872 | 0.907 | 0.851 | 0.963 | 0.905 | 0.829 | 0.982 |
| SIGLEC1, ISG15, HERC6, MMP8 | | | | | | | | | |
| IFIT1, SIGLEC1, ISG15, HERC6, | 0.863 | 0.853 | 0.873 | 0.906 | 0.845 | 0.968 | 0.966 | 0.930 | 1 |
| IL1R2, OLAH, RETN, MMP8 | | | | | | | | | |
| IFIT1, IFI44L, ISG15, HERC6, | 0.863 | 0.854 | 0.872 | 0.909 | 0.849 | 0.968 | 0.933 | 0.854 | 1 |
| IL1R2, OLAH, FAM20A, RETN | | | | | | | | | |
| IFIT1, IFI44L, HERC6, IL1R2, | 0.863 | 0.853 | 0.872 | 0.911 | 0.851 | 0.971 | 0.942 | 0.869 | 1 |
| OLAH, FAM20A, RETN, MMP8 | | | | | | | | | |
| RSAD2, IFI27, IFIT1, IFI44L, | 0.863 | 0.854 | 0.872 | 0.915 | 0.859 | 0.971 | 0.951 | 0.892 | 1 |
| SIGLEC1, ISG15, SLC1A2, RETN | | | | | | | | | |
| IFIT1, IFI44L, ISG15, HERC6, | 0.863 | 0.853 | 0.873 | 0.910 | 0.850 | 0.970 | 0.940 | 0.862 | 1 |
| IL1R2, FAM20A, RETN, MMP8 | | | | | | | | | |
| OAS1, ISG15, HERC6, IL1R2, | 0.863 | 0.853 | 0.873 | 0.910 | 0.851 | 0.970 | 0.943 | 0.869 | 1 |
| OLAH, FAM20A, RETN, MMP8 | | | | | | | | | |
| RSAD2, OAS1, IFI44L, SLC1A2, | 0.863 | 0.853 | 0.873 | 0.917 | 0.865 | 0.969 | 0.928 | 0.848 | 1 |
| IL1R2, OLAH, FAM20A, MMP8 | | | | | | | | | |
| OAS1, IFIT1, ISG15, HERC6, | 0.863 | 0.853 | 0.873 | 0.910 | 0.849 | 0.970 | 0.938 | 0.859 | 1 |
| IL1R2, FAM20A, RETN, MMP8 | | | | | | | | | |
| RSAD2, OAS1, IFIT1, SIGLEC1, | 0.863 | 0.853 | 0.873 | 0.914 | 0.856 | 0.972 | 0.952 | 0.884 | 1 |
| SLC1A2, FAM20A, RETN, MMP8 | | | | | | | | | |
| RSAD2, IFI44L, SIGLEC1, HERC6, | 0.863 | 0.853 | 0.873 | 0.913 | 0.855 | 0.971 | 0.949 | 0.877 | 1 |
| SLC1A2, FAM20A, RETN, MMP8 | | | | | | | | | |
| RSAD2, OAS1, IFI44L, ISG15, | 0.863 | 0.853 | 0.873 | 0.911 | 0.853 | 0.969 | 0.928 | 0.844 | 1 |
| HERC6, IL1R2, FAM20A, MMP8 | | | | | | | | | |
| IFIT1, IFI44L, ISG15, HERC6, | 0.863 | 0.853 | 0.873 | 0.918 | 0.864 | 0.971 | 0.918 | 0.833 | 1 |
| SLC1A2, OLAH, FAM20A, RETN | | | | | | | | | |
| RSAD2, OAS1, IFIT1, IFI44L, | 0.863 | 0.853 | 0.873 | 0.908 | 0.847 | 0.969 | 0.945 | 0.876 | 1 |
| IL1R2, OLAH, FAM20A, MMP8 | | | | | | | | | |
| RSAD2, IFI27, IFIT1, IFI44L, | 0.863 | 0.853 | 0.873 | 0.911 | 0.850 | 0.972 | 0.950 | 0.892 | 1 |
| SIGLEC1, ISG15, RETN, MMP8 | | | | | | | | | |
| RSAD2, IFI44L, SIGLEC1, ISG15, | 0.863 | 0.853 | 0.873 | 0.916 | 0.858 | 0.973 | 0.946 | 0.884 | 1 |
| SLC1A2, OLAH, FAM20A, RETN | | | | | | | | | |
| IFI27, OAS1, IFIT1, SIGLEC1, | 0.863 | 0.854 | 0.872 | 0.913 | 0.860 | 0.965 | 0.915 | 0.843 | 0.987 |
| ISG15, HERC6, SLC1A2, MMP8 | | | | | | | | | |
| RSAD2, OAS1, IFIT1, ISG15, | 0.863 | 0.853 | 0.873 | 0.908 | 0.848 | 0.968 | 0.937 | 0.860 | 1 |
| HERC6, IL1R2, FAM20A, MMP8 | | | | | | | | | |
| RSAD2, OAS1, IFIT1, IFI44L, | 0.863 | 0.853 | 0.872 | 0.908 | 0.847 | 0.968 | 0.932 | 0.854 | 1 |
| HERC6, IL1R2, OLAH, FAM20A | | | | | | | | | |
| OAS1, IFIT1, ISG15, HERC6, | 0.863 | 0.853 | 0.873 | 0.907 | 0.848 | 0.967 | 0.943 | 0.871 | 1 |
| IL1R2, OLAH, FAM20A, MMP8 | | | | | | | | | |
| RSAD2, OAS1, IFIT1, IFI44L, | 0.863 | 0.853 | 0.873 | 0.917 | 0.863 | 0.970 | 0.912 | 0.825 | 0.999 |
| HERC6, SLC1A2, OLAH, FAM20A | | | | | | | | | |
| OAS1, IFIT1, IFI44L, SIGLEC1, | 0.863 | 0.853 | 0.872 | 0.907 | 0.848 | 0.966 | 0.952 | 0.907 | 0.998 |
| ISG15, HERC6, IL1R2, RETN | | | | | | | | | |
| RSAD2, IFI44L, SIGLEC1, ISG15, | 0.863 | 0.853 | 0.873 | 0.908 | 0.847 | 0.970 | 0.949 | 0.892 | 1 |
| HERC6, OLAH, FAM20A, RETN | | | | | | | | | |
| IFI27, IFIT1, IFI44L, ISG15, | 0.863 | 0.852 | 0.873 | 0.914 | 0.857 | 0.970 | 0.915 | 0.826 | 1 |
| HERC6, SLC1A2, FAM20A, MMP8 | | | | | | | | | |
| OAS1, IFIT1, HERC6, IL1R2, | 0.863 | 0.853 | 0.872 | 0.908 | 0.846 | 0.969 | 0.942 | 0.868 | 1 |
| OLAH, FAM20A, RETN, MMP8 | | | | | | | | | |
| RSAD2, ISG15, HERC6, IL1R2, | 0.863 | 0.852 | 0.873 | 0.909 | 0.849 | 0.969 | 0.938 | 0.861 | 1 |
| OLAH, FAM20A, RETN, MMP8 | | | | | | | | | |
| RSAD2, IFIT1, IFI44L, SLC1A2, | 0.863 | 0.853 | 0.872 | 0.917 | 0.865 | 0.969 | 0.932 | 0.854 | 1 |
| IL1R2, OLAH, FAM20A, MMP8 | | | | | | | | | |
| OAS1, IFI44L, ISG15, HERC6, | 0.863 | 0.852 | 0.873 | 0.910 | 0.851 | 0.968 | 0.941 | 0.867 | 1 |
| IL1R2, OLAH, FAM20A, MMP8 | | | | | | | | | |
| RSAD2, OAS1, IFI44L, HERC6, | 0.862 | 0.852 | 0.873 | 0.916 | 0.862 | 0.970 | 0.920 | 0.840 | 0.999 |
| SLC1A2, OLAH, FAM20A, MMP8 | | | | | | | | | |
| RSAD2, OAS1, IFIT1, IFI44L, | 0.862 | 0.853 | 0.872 | 0.909 | 0.850 | 0.967 | 0.930 | 0.850 | 1 |
| ISG15, HERC6, IL1R2, FAM20A | | | | | | | | | |
| RSAD2, IFI44L, ISG15, HERC6, | 0.862 | 0.852 | 0.873 | 0.913 | 0.855 | 0.970 | 0.932 | 0.853 | 1 |
| IL1R2, OLAH, FAM20A, MMP8 | | | | | | | | | |
| RSAD2, OAS1, IFI44L, SIGLEC1, | 0.862 | 0.852 | 0.873 | 0.915 | 0.857 | 0.973 | 0.945 | 0.883 | 1 |
| SLC1A2, OLAH, FAM20A, RETN | | | | | | | | | |
| IFIT1, IFI44L, SIGLEC1, HERC6, | 0.862 | 0.852 | 0.872 | 0.913 | 0.858 | 0.969 | 0.950 | 0.904 | 0.996 |
| SLC1A2, OLAH, RETN, MMP8 | | | | | | | | | |
| IFI44L, SIGLEC1, ISG15, HERC6, | 0.862 | 0.852 | 0.873 | 0.905 | 0.842 | 0.968 | 0.953 | 0.880 | 1 |
| SLC1A2, FAM20A, RETN, MMP8 | | | | | | | | | |
| OAS1, IFI44L, SIGLEC1, ISG15, | 0.862 | 0.852 | 0.873 | 0.907 | 0.844 | 0.970 | 0.954 | 0.899 | 1 |
| HERC6, OLAH, FAM20A, RETN | | | | | | | | | |
| RSAD2, OAS1, IFIT1, SIGLEC1, | 0.862 | 0.852 | 0.872 | 0.909 | 0.848 | 0.969 | 0.946 | 0.872 | 1 |
| ISG15, SLC1A2, FAM20A, MMP8 | | | | | | | | | |
| IFIT1, SIGLEC1, HERC6, SLC1A2, | 0.862 | 0.853 | 0.872 | 0.916 | 0.862 | 0.970 | 0.962 | 0.924 | 1 |
| IL1R2, OLAH, RETN, MMP8 | | | | | | | | | |
| RSAD2, IFI27, OAS1, IFI44L, | 0.862 | 0.854 | 0.871 | 0.920 | 0.872 | 0.967 | 0.930 | 0.862 | 0.999 |
| ISG15, SLC1A2, IL1R2, MMP8 | | | | | | | | | |
| RSAD2, OAS1, SIGLEC1, ISG15, | 0.862 | 0.852 | 0.873 | 0.909 | 0.852 | 0.966 | 0.940 | 0.864 | 1 |
| HERC6, SLC1A2, FAM20A, MMP8 | | | | | | | | | |
| OAS1, IFIT1, IFI44L, SIGLEC1, | 0.862 | 0.852 | 0.872 | 0.901 | 0.836 | 0.967 | 0.952 | 0.908 | 0.997 |
| HERC6, OLAH, RETN, MMP8 | | | | | | | | | |
| OAS1, IFI44L, ISG15, HERC6, | 0.862 | 0.851 | 0.873 | 0.912 | 0.858 | 0.966 | 0.924 | 0.839 | 1 |
| SLC1A2, IL1R2, OLAH, FAM20A | | | | | | | | | |
| RSAD2, OAS1, IFIT1, IFI44L, | 0.862 | 0.853 | 0.872 | 0.904 | 0.841 | 0.966 | 0.955 | 0.911 | 0.999 |
| SIGLEC1, HERC6, IL1R2, RETN | | | | | | | | | |
| IFI27, OAS1, IFIT1, IFI44L, | 0.862 | 0.854 | 0.871 | 0.912 | 0.859 | 0.965 | 0.917 | 0.847 | 0.988 |
| SIGLEC1, HERC6, SLC1A2, MMP8 | | | | | | | | | |
| RSAD2, OAS1, IFI44L, SIGLEC1, | 0.862 | 0.852 | 0.872 | 0.907 | 0.845 | 0.970 | 0.949 | 0.892 | 1 |
| HERC6, OLAH, FAM20A, RETN | | | | | | | | | |
| RSAD2, IFIT1, IFI44L, SIGLEC1, | 0.862 | 0.853 | 0.872 | 0.913 | 0.854 | 0.972 | 0.936 | 0.863 | 1 |
| ISG15, SLC1A2, FAM20A, RETN | | | | | | | | | |
| RSAD2, OAS1, IFIT1, SIGLEC1, | 0.862 | 0.853 | 0.872 | 0.905 | 0.845 | 0.966 | 0.958 | 0.914 | 1 |
| ISG15, HERC6, IL1R2, RETN | | | | | | | | | |
| RSAD2, OAS1, HERC6, SLC1A2, | 0.862 | 0.853 | 0.872 | 0.914 | 0.862 | 0.966 | 0.927 | 0.840 | 1 |
| IL1R2, OLAH, FAM20A, MMP8 | | | | | | | | | |
| OAS1, IFIT1, SIGLEC1, ISG15, | 0.862 | 0.852 | 0.872 | 0.904 | 0.841 | 0.966 | 0.959 | 0.918 | 0.999 |
| HERC6, OLAH, RETN, MMP8 | | | | | | | | | |
| OAS1, IFIT1, IFI44L, SIGLEC1, | 0.862 | 0.852 | 0.872 | 0.915 | 0.860 | 0.969 | 0.937 | 0.881 | 0.993 |
| HERC6, SLC1A2, OLAH, RETN | | | | | | | | | |
| OAS1, IFIT1, HERC6, SLC1A2, | 0.862 | 0.852 | 0.872 | 0.916 | 0.865 | 0.968 | 0.932 | 0.850 | 1 |
| IL1R2, OLAH, FAM20A, MMP8 | | | | | | | | | |
| RSAD2, OAS1, IFIT1, ISG15, | 0.862 | 0.852 | 0.872 | 0.907 | 0.847 | 0.966 | 0.937 | 0.862 | 1 |
| HERC6, IL1R2, OLAH, FAM20A | | | | | | | | | |
| OAS1, IFIT1, SIGLEC1, HERC6, | 0.862 | 0.853 | 0.872 | 0.911 | 0.856 | 0.966 | 0.963 | 0.925 | 1 |
| SLC1A2, IL1R2, RETN, MMP8 | | | | | | | | | |
| RSAD2, IFIT1, IFI44L, ISG15, | 0.862 | 0.852 | 0.872 | 0.916 | 0.863 | 0.970 | 0.916 | 0.831 | 1 |
| HERC6, SLC1A2, OLAH, FAM20A | | | | | | | | | |
| RSAD2, OAS1, IFIT1, SIGLEC1, | 0.862 | 0.852 | 0.872 | 0.910 | 0.849 | 0.970 | 0.945 | 0.873 | 1 |
| ISG15, SLC1A2, FAM20A, RETN | | | | | | | | | |
| IFIT1, ISG15, HERC6, IL1R2, | 0.862 | 0.852 | 0.872 | 0.909 | 0.848 | 0.969 | 0.943 | 0.871 | 1 |
| OLAH, FAM20A, RETN, MMP8 | | | | | | | | | |
| IFI44L, ISG15, HERC6, SLC1A2, | 0.862 | 0.852 | 0.872 | 0.914 | 0.857 | 0.971 | 0.935 | 0.858 | 1 |
| OLAH, FAM20A, RETN, MMP8 | | | | | | | | | |
| IFIT1, IFI44L, ISG15, HERC6, | 0.862 | 0.852 | 0.872 | 0.908 | 0.845 | 0.972 | 0.943 | 0.876 | 1 |
| OLAH, FAM20A, RETN, MMP8 | | | | | | | | | |
| IFIT1, IFI44L, SIGLEC1, ISG15, | 0.862 | 0.852 | 0.872 | 0.913 | 0.858 | 0.968 | 0.941 | 0.889 | 0.993 |
| HERC6, SLC1A2, OLAH, MMP8 | | | | | | | | | |
| RSAD2, IFI27, IFIT1, IFI44L, | 0.862 | 0.852 | 0.872 | 0.910 | 0.848 | 0.973 | 0.939 | 0.859 | 1 |
| ISG15, SLC1A2, FAM20A, RETN | | | | | | | | | |
| RSAD2, OAS1, IFIT1, SLC1A2, | 0.862 | 0.853 | 0.871 | 0.917 | 0.865 | 0.969 | 0.935 | 0.855 | 1 |
| IL1R2, OLAH, FAM20A, MMP8 | | | | | | | | | |
| OAS1, IFIT1, IFI44L, ISG15, | 0.862 | 0.852 | 0.872 | 0.909 | 0.850 | 0.967 | 0.932 | 0.853 | 1 |
| HERC6, IL1R2, OLAH, FAM20A | | | | | | | | | |
| RSAD2, IFIT1, ISG15, HERC6, | 0.862 | 0.852 | 0.872 | 0.911 | 0.851 | 0.970 | 0.937 | 0.859 | 1 |
| IL1R2, FAM20A, RETN, MMP8 | | | | | | | | | |
| OAS1, IFIT1, IFI44L, HERC6, | 0.862 | 0.852 | 0.872 | 0.917 | 0.863 | 0.970 | 0.913 | 0.826 | 1 |
| SLC1A2, OLAH, FAM20A, RETN | | | | | | | | | |
| OAS1, SIGLEC1, HERC6, SLC1A2, | 0.862 | 0.852 | 0.872 | 0.915 | 0.860 | 0.969 | 0.962 | 0.923 | 1 |
| IL1R2, OLAH, RETN, MMP8 | | | | | | | | | |
| RSAD2, OAS1, ISG15, HERC6, | 0.862 | 0.851 | 0.872 | 0.906 | 0.845 | 0.966 | 0.937 | 0.862 | 1 |
| IL1R2, OLAH, FAM20A, RETN | | | | | | | | | |
| RSAD2, OAS1, IFIT1, HERC6, | 0.862 | 0.852 | 0.872 | 0.908 | 0.847 | 0.970 | 0.941 | 0.862 | 1 |
| IL1R2, FAM20A, RETN, MMP8 | | | | | | | | | |
| IFIT1, IFI44L, SIGLEC1, HERC6, | 0.862 | 0.852 | 0.872 | 0.904 | 0.841 | 0.967 | 0.964 | 0.926 | 1 |
| IL1R2, OLAH, RETN, MMP8 | | | | | | | | | |
| RSAD2, IFI27, IFIT1, ISG15, | 0.862 | 0.852 | 0.872 | 0.915 | 0.859 | 0.972 | 0.936 | 0.868 | 1 |
| HERC6, SLC1A2, RETN, MMP8 | | | | | | | | | |
| IFI27, IFIT1, IFI44L, ISG15, | 0.862 | 0.851 | 0.873 | 0.913 | 0.852 | 0.975 | 0.940 | 0.861 | 1 |
| SLC1A2, FAM20A, RETN, MMP8 | | | | | | | | | |
| RSAD2, IFIT1, HERC6, SLC1A2, | 0.862 | 0.852 | 0.872 | 0.916 | 0.865 | 0.968 | 0.932 | 0.850 | 1 |
| IL1R2, OLAH, FAM20A, MMP8 | | | | | | | | | |
| OAS1, IFIT1, ISG15, HERC6, | 0.862 | 0.852 | 0.872 | 0.906 | 0.845 | 0.966 | 0.936 | 0.860 | 1 |
| IL1R2, OLAH, FAM20A, RETN | | | | | | | | | |
| RSAD2, OAS1, IFIT1, ISG15, | 0.862 | 0.852 | 0.872 | 0.907 | 0.848 | 0.967 | 0.934 | 0.851 | 1 |
| HERC6, IL1R2, FAM20A, RETN | | | | | | | | | |
| RSAD2, IFIT1, IFI44L, HERC6, | 0.862 | 0.852 | 0.872 | 0.910 | 0.851 | 0.968 | 0.933 | 0.854 | 1 |
| IL1R2, OLAH, FAM20A, MMP8 | | | | | | | | | |
| OAS1, IFIT1, SIGLEC1, HERC6, | 0.862 | 0.852 | 0.872 | 0.913 | 0.860 | 0.966 | 0.961 | 0.922 | 1 |
| SLC1A2, IL1R2, OLAH, MMP8 | | | | | | | | | |
| RSAD2, IFI27, OAS1, IFI44L, | 0.862 | 0.851 | 0.872 | 0.911 | 0.850 | 0.973 | 0.940 | 0.861 | 1 |
| ISG15, SLC1A2, FAM20A, RETN | | | | | | | | | |
| IFI27, IFIT1, IFI44L, ISG15, | 0.862 | 0.852 | 0.871 | 0.912 | 0.854 | 0.971 | 0.928 | 0.855 | 1 |
| HERC6, SLC1A2, RETN, MMP8 | | | | | | | | | |
| RSAD2, IFIT1, ISG15, HERC6, | 0.862 | 0.852 | 0.872 | 0.907 | 0.848 | 0.966 | 0.945 | 0.874 | 1 |
| IL1R2, OLAH, FAM20A, MMP8 | | | | | | | | | |
| IFIT1, SIGLEC1, ISG15, HERC6, | 0.862 | 0.852 | 0.871 | 0.913 | 0.860 | 0.966 | 0.961 | 0.922 | 1.000 |
| SLC1A2, IL1R2, OLAH, MMP8 | | | | | | | | | |
| OAS1, IFI44L, SIGLEC1, ISG15, | 0.862 | 0.852 | 0.871 | 0.904 | 0.842 | 0.966 | 0.963 | 0.925 | 1 |
| HERC6, IL1R2, RETN, MMP8 | | | | | | | | | |
| RSAD2, OAS1, SIGLEC1, HERC6, | 0.862 | 0.851 | 0.872 | 0.905 | 0.843 | 0.967 | 0.965 | 0.929 | 1 |
| IL1R2, OLAH, RETN, MMP8 | | | | | | | | | |
| RSAD2, IFIT1, IFI44L, HERC6, | 0.862 | 0.852 | 0.872 | 0.918 | 0.866 | 0.969 | 0.916 | 0.828 | 1 |
| SLC1A2, IL1R2, OLAH, FAM20A | | | | | | | | | |
| RSAD2, IFI27, IFIT1, IFI44L, | 0.862 | 0.851 | 0.872 | 0.913 | 0.855 | 0.970 | 0.918 | 0.833 | 1 |
| ISG15, HERC6, SLC1A2, FAM20A | | | | | | | | | |
| IFIT1, IFI44L, ISG15, SLC1A2, | 0.862 | 0.852 | 0.871 | 0.913 | 0.856 | 0.970 | 0.924 | 0.848 | 1.000 |
| OLAH, FAM20A, RETN, MMP8 | | | | | | | | | |
| OAS1, IFIT1, SIGLEC1, ISG15, | 0.862 | 0.852 | 0.871 | 0.906 | 0.846 | 0.966 | 0.952 | 0.907 | 0.997 |
| HERC6, IL1R2, OLAH, RETN | | | | | | | | | |
| IFI27, IFIT1, IFI44L, SIGLEC1, | 0.862 | 0.852 | 0.871 | 0.914 | 0.858 | 0.971 | 0.950 | 0.891 | 1 |
| ISG15, SLC1A2, RETN, MMP8 | | | | | | | | | |
| RSAD2, OAS1, SIGLEC1, ISG15, | 0.862 | 0.851 | 0.872 | 0.907 | 0.846 | 0.967 | 0.970 | 0.936 | 1 |
| HERC6, IL1R2, RETN, MMP8 | | | | | | | | | |
| RSAD2, IFIT1, IFI44L, ISG15, | 0.861 | 0.852 | 0.871 | 0.910 | 0.851 | 0.968 | 0.930 | 0.853 | 1 |
| HERC6, IL1R2, OLAH, FAM20A | | | | | | | | | |
| RSAD2, IFI27, OAS1, SIGLEC1, | 0.861 | 0.852 | 0.871 | 0.913 | 0.859 | 0.966 | 0.927 | 0.858 | 0.996 |
| ISG15, HERC6, SLC1A2, MMP8 | | | | | | | | | |
| RSAD2, OAS1, IFIT1, IFI44L, | 0.861 | 0.852 | 0.871 | 0.915 | 0.860 | 0.971 | 0.923 | 0.847 | 0.999 |
| SLC1A2, OLAH, FAM20A, MMP8 | | | | | | | | | |
| RSAD2, IFI27, OAS1, IFIT1, | 0.861 | 0.851 | 0.872 | 0.910 | 0.848 | 0.972 | 0.940 | 0.861 | 1 |
| IFI44L, SLC1A2, FAM20A, RETN | | | | | | | | | |
| IFIT1, IFI44L, SIGLEC1, ISG15, | 0.861 | 0.851 | 0.871 | 0.905 | 0.841 | 0.968 | 0.959 | 0.918 | 0.999 |
| HERC6, OLAH, RETN, MMP8 | | | | | | | | | |
| OAS1, SIGLEC1, ISG15, HERC6, | 0.861 | 0.852 | 0.871 | 0.912 | 0.859 | 0.965 | 0.961 | 0.922 | 1 |
| SLC1A2, IL1R2, OLAH, MMP8 | | | | | | | | | |
| OAS1, SIGLEC1, ISG15, HERC6, | 0.861 | 0.851 | 0.872 | 0.915 | 0.859 | 0.970 | 0.962 | 0.921 | 1 |
| SLC1A2, OLAH, RETN, MMP8 | | | | | | | | | |
| RSAD2, OAS1, IFI44L, SIGLEC1, | 0.861 | 0.851 | 0.871 | 0.910 | 0.854 | 0.967 | 0.940 | 0.866 | 1 |
| ISG15, HERC6, SLC1A2, FAM20A | | | | | | | | | |
| RSAD2, IFIT1, SIGLEC1, ISG15, | 0.861 | 0.851 | 0.871 | 0.913 | 0.854 | 0.971 | 0.950 | 0.881 | 1 |
| SLC1A2, FAM20A, RETN, MMP8 | | | | | | | | | |
| OAS1, IFI44L, SIGLEC1, ISG15, | 0.861 | 0.851 | 0.872 | 0.911 | 0.853 | 0.968 | 0.946 | 0.876 | 1 |
| HERC6, SLC1A2, FAM20A, RETN | | | | | | | | | |
| IFIT1, IFI44L, SLC1A2, IL1R2, | 0.861 | 0.852 | 0.871 | 0.916 | 0.862 | 0.969 | 0.934 | 0.857 | 1 |
| OLAH, FAM20A, RETN, MMP8 | | | | | | | | | |
| RSAD2, IFI27, IFI44L, ISG15, | 0.861 | 0.850 | 0.873 | 0.913 | 0.856 | 0.970 | 0.925 | 0.842 | 1 |
| HERC6, SLC1A2, FAM20A, MMP8 | | | | | | | | | |
| RSAD2, IFI44L, SIGLEC1, ISG15, | 0.861 | 0.851 | 0.871 | 0.908 | 0.844 | 0.972 | 0.963 | 0.920 | 1 |
| OLAH, FAM20A, RETN, MMP8 | | | | | | | | | |
| RSAD2, IFI44L, ISG15, HERC6, | 0.861 | 0.850 | 0.872 | 0.912 | 0.858 | 0.967 | 0.925 | 0.841 | 1 |
| SLC1A2, IL1R2, OLAH, FAM20A | | | | | | | | | |
| IFIT1, ISG15, HERC6, SLC1A2, | 0.861 | 0.851 | 0.871 | 0.916 | 0.864 | 0.967 | 0.932 | 0.850 | 1 |
| IL1R2, OLAH, FAM20A, MMP8 | | | | | | | | | |
| OAS1, IFI44L, ISG15, HERC6, | 0.861 | 0.851 | 0.871 | 0.910 | 0.850 | 0.970 | 0.936 | 0.856 | 1 |
| IL1R2, FAM20A, RETN, MMP8 | | | | | | | | | |
| RSAD2, IFIT1, IFI44L, SIGLEC1, | 0.861 | 0.852 | 0.871 | 0.903 | 0.840 | 0.966 | 0.964 | 0.926 | 1 |
| HERC6, IL1R2, RETN, MMP8 | | | | | | | | | |
| OAS1, IFIT1, IFI44L, SIGLEC1, | 0.861 | 0.852 | 0.871 | 0.905 | 0.844 | 0.966 | 0.942 | 0.891 | 0.994 |
| ISG15, HERC6, OLAH, RETN | | | | | | | | | |
| RSAD2, IFI44L, SIGLEC1, ISG15, | 0.861 | 0.851 | 0.872 | 0.912 | 0.855 | 0.970 | 0.939 | 0.865 | 1 |
| HERC6, SLC1A2, FAM20A, RETN | | | | | | | | | |
| RSAD2, IFI27, IFI44L, ISG15, | 0.861 | 0.852 | 0.871 | 0.915 | 0.859 | 0.972 | 0.937 | 0.870 | 1 |
| HERC6, SLC1A2, RETN, MMP8 | | | | | | | | | |
| OAS1, IFIT1, SIGLEC1, ISG15, | 0.861 | 0.851 | 0.871 | 0.915 | 0.860 | 0.969 | 0.942 | 0.890 | 0.995 |
| HERC6, SLC1A2, OLAH, RETN | | | | | | | | | |
| RSAD2, IFIT1, SIGLEC1, HERC6, | 0.861 | 0.852 | 0.871 | 0.912 | 0.859 | 0.966 | 0.962 | 0.924 | 1 |
| SLC1A2, IL1R2, OLAH, MMP8 | | | | | | | | | |
| OAS1, IFIT1, IFI44L, HERC6, | 0.861 | 0.851 | 0.872 | 0.916 | 0.863 | 0.968 | 0.917 | 0.830 | 1 |
| SLC1A2, IL1R2, OLAH, FAM20A | | | | | | | | | |
| OAS1, IFIT1, IFI44L, SLC1A2, | 0.861 | 0.851 | 0.871 | 0.917 | 0.866 | 0.969 | 0.929 | 0.850 | 1 |
| IL1R2, OLAH, FAM20A, MMP8 | | | | | | | | | |
| OAS1, IFIT1, IFI44L, SIGLEC1, | 0.861 | 0.851 | 0.871 | 0.913 | 0.858 | 0.967 | 0.940 | 0.888 | 0.993 |
| HERC6, SLC1A2, OLAH, MMP8 | | | | | | | | | |
| OAS1, IFI44L, ISG15, SLC1A2, | 0.861 | 0.851 | 0.871 | 0.916 | 0.863 | 0.968 | 0.926 | 0.843 | 1 |
| IL1R2, OLAH, FAM20A, MMP8 | | | | | | | | | |
| RSAD2, OAS1, IFIT1, ISG15, | 0.861 | 0.851 | 0.871 | 0.908 | 0.846 | 0.970 | 0.945 | 0.873 | 1 |
| IL1R2, OLAH, FAM20A, MMP8 | | | | | | | | | |
| RSAD2, IFI27, OAS1, IFI44L, | 0.861 | 0.852 | 0.870 | 0.914 | 0.861 | 0.967 | 0.928 | 0.858 | 0.998 |
| SIGLEC1, HERC6, SLC1A2, MMP8 | | | | | | | | | |
| IFIT1, SIGLEC1, ISG15, HERC6, | 0.861 | 0.851 | 0.871 | 0.914 | 0.859 | 0.969 | 0.952 | 0.907 | 0.997 |
| SLC1A2, OLAH, RETN, MMP8 | | | | | | | | | |
| OAS1, IFI44L, HERC6, SLC1A2, | 0.861 | 0.851 | 0.871 | 0.918 | 0.863 | 0.973 | 0.929 | 0.851 | 1 |
| OLAH, FAM20A, RETN, MMP8 | | | | | | | | | |
| RSAD2, IFI44L, HERC6, SLC1A2, | 0.861 | 0.851 | 0.871 | 0.917 | 0.862 | 0.971 | 0.925 | 0.845 | 1 |
| OLAH, FAM20A, RETN, MMP8 | | | | | | | | | |
| IFI44L, ISG15, HERC6, SLC1A2, | 0.861 | 0.851 | 0.871 | 0.914 | 0.860 | 0.968 | 0.929 | 0.846 | 1 |
| IL1R2, OLAH, FAM20A, RETN | | | | | | | | | |
| RSAD2, OAS1, IFIT1, SIGLEC1, | 0.861 | 0.851 | 0.871 | 0.912 | 0.857 | 0.967 | 0.951 | 0.905 | 0.998 |
| HERC6, SLC1A2, OLAH, MMP8 | | | | | | | | | |
| RSAD2, OAS1, IFI44L, HERC6, | 0.861 | 0.850 | 0.872 | 0.915 | 0.862 | 0.968 | 0.923 | 0.838 | 1 |
| SLC1A2, IL1R2, OLAH, FAM20A | | | | | | | | | |
| IFI27, OAS1, IFIT1, IFI44L, | 0.861 | 0.850 | 0.872 | 0.913 | 0.852 | 0.974 | 0.939 | 0.859 | 1 |
| SLC1A2, FAM20A, RETN, MMP8 | | | | | | | | | |
| RSAD2, IFI44L, ISG15, HERC6, | 0.861 | 0.851 | 0.871 | 0.910 | 0.850 | 0.969 | 0.938 | 0.867 | 1 |
| IL1R2, OLAH, FAM20A, RETN | | | | | | | | | |
| RSAD2, OAS1, ISG15, HERC6, | 0.861 | 0.850 | 0.872 | 0.912 | 0.858 | 0.966 | 0.927 | 0.841 | 1 |
| SLC1A2, IL1R2, OLAH, FAM20A | | | | | | | | | |
| OAS1, IFIT1, IFI44L, ISG15, | 0.861 | 0.851 | 0.871 | 0.911 | 0.852 | 0.969 | 0.935 | 0.856 | 1 |
| HERC6, IL1R2, FAM20A, RETN | | | | | | | | | |
| OAS1, SIGLEC1, ISG15, HERC6, | 0.861 | 0.851 | 0.871 | 0.906 | 0.845 | 0.967 | 0.966 | 0.930 | 1 |
| IL1R2, OLAH, RETN, MMP8 | | | | | | | | | |
| IFIT1, HERC6, SLC1A2, IL1R2, | 0.861 | 0.851 | 0.871 | 0.915 | 0.862 | 0.969 | 0.934 | 0.853 | 1 |
| OLAH, FAM20A, RETN, MMP8 | | | | | | | | | |
| RSAD2, IFI27, OAS1, IFI44L, | 0.861 | 0.850 | 0.872 | 0.912 | 0.856 | 0.969 | 0.925 | 0.842 | 1 |
| HERC6, SLC1A2, FAM20A, MMP8 | | | | | | | | | |
| OAS1, IFI44L, ISG15, HERC6, | 0.861 | 0.850 | 0.872 | 0.914 | 0.858 | 0.969 | 0.927 | 0.850 | 1 |
| SLC1A2, OLAH, FAM20A, MMP8 | | | | | | | | | |
| OAS1, IFIT1, IFI44L, HERC6, | 0.861 | 0.851 | 0.871 | 0.909 | 0.848 | 0.970 | 0.940 | 0.861 | 1 |
| IL1R2, FAM20A, RETN, MMP8 | | | | | | | | | |
| RSAD2, OAS1, IFIT1, IFI44L, | 0.861 | 0.851 | 0.871 | 0.914 | 0.855 | 0.972 | 0.936 | 0.863 | 1 |
| SIGLEC1, SLC1A2, FAM20A, RETN | | | | | | | | | |
| RSAD2, OAS1, IFIT1, SIGLEC1, | 0.861 | 0.851 | 0.871 | 0.913 | 0.859 | 0.967 | 0.961 | 0.921 | 1 |
| HERC6, SLC1A2, IL1R2, OLAH | | | | | | | | | |
| OAS1, IFIT1, IFI44L, ISG15, | 0.861 | 0.851 | 0.871 | 0.906 | 0.845 | 0.967 | 0.933 | 0.863 | 1 |
| HERC6, OLAH, FAM20A, RETN | | | | | | | | | |
| RSAD2, OAS1, SIGLEC1, HERC6, | 0.861 | 0.851 | 0.871 | 0.912 | 0.855 | 0.970 | 0.962 | 0.921 | 1 |
| SLC1A2, OLAH, RETN, MMP8 | | | | | | | | | |
| RSAD2, ISG15, HERC6, SLC1A2, | 0.861 | 0.851 | 0.870 | 0.914 | 0.861 | 0.966 | 0.926 | 0.837 | 1 |
| IL1R2, OLAH, FAM20A, MMP8 | | | | | | | | | |
| RSAD2, IFI44L, ISG15, HERC6, | 0.861 | 0.850 | 0.871 | 0.916 | 0.862 | 0.970 | 0.922 | 0.843 | 1 |
| SLC1A2, OLAH, FAM20A, MMP8 | | | | | | | | | |
| RSAD2, OAS1, IFIT1, SIGLEC1, | 0.861 | 0.851 | 0.871 | 0.912 | 0.856 | 0.969 | 0.942 | 0.887 | 0.998 |
| HERC6, SLC1A2, OLAH, RETN | | | | | | | | | |
| RSAD2, IFIT1, SIGLEC1, ISG15, | 0.861 | 0.851 | 0.870 | 0.906 | 0.844 | 0.967 | 0.963 | 0.925 | 1 |
| HERC6, IL1R2, RETN, MMP8 | | | | | | | | | |
| RSAD2, OAS1, IFI44L, ISG15, | 0.861 | 0.850 | 0.871 | 0.911 | 0.853 | 0.970 | 0.936 | 0.860 | 1 |
| HERC6, IL1R2, FAM20A, RETN | | | | | | | | | |
| OAS1, ISG15, HERC6, SLC1A2, | 0.861 | 0.850 | 0.871 | 0.914 | 0.863 | 0.966 | 0.927 | 0.841 | 1 |
| IL1R2, OLAH, FAM20A, MMP8 | | | | | | | | | |
| OAS1, IFI44L, HERC6, IL1R2, | 0.861 | 0.851 | 0.871 | 0.913 | 0.852 | 0.973 | 0.945 | 0.872 | 1 |
| OLAH, FAM20A, RETN, MMP8 | | | | | | | | | |
| RSAD2, SIGLEC1, ISG15, HERC6, | 0.861 | 0.851 | 0.870 | 0.912 | 0.859 | 0.966 | 0.962 | 0.924 | 1 |
| SLC1A2, IL1R2, OLAH, MMP8 | | | | | | | | | |
| RSAD2, OAS1, IFI44L, ISG15, | 0.861 | 0.850 | 0.871 | 0.907 | 0.846 | 0.969 | 0.942 | 0.873 | 1 |
| IL1R2, OLAH, FAM20A, MMP8 | | | | | | | | | |
| RSAD2, OAS1, SIGLEC1, HERC6, | 0.861 | 0.851 | 0.871 | 0.912 | 0.859 | 0.966 | 0.963 | 0.925 | 1 |
| SLC1A2, IL1R2, OLAH, MMP8 | | | | | | | | | |
| RSAD2, IFIT1, IFI44L, ISG15, | 0.861 | 0.851 | 0.870 | 0.912 | 0.855 | 0.970 | 0.922 | 0.845 | 0.998 |
| SLC1A2, OLAH, FAM20A, MMP8 | | | | | | | | | |
| IFIT1, IFI44L, HERC6, SLC1A2, | 0.861 | 0.850 | 0.871 | 0.917 | 0.864 | 0.969 | 0.920 | 0.833 | 1 |
| IL1R2, OLAH, FAM20A, RETN | | | | | | | | | |
| RSAD2, IFI27, IFIT1, IFI44L, | 0.861 | 0.850 | 0.871 | 0.915 | 0.854 | 0.975 | 0.940 | 0.861 | 1 |
| SLC1A2, FAM20A, RETN, MMP8 | | | | | | | | | |
| OAS1, IFIT1, SIGLEC1, ISG15, | 0.861 | 0.851 | 0.871 | 0.912 | 0.859 | 0.966 | 0.953 | 0.907 | 0.999 |
| HERC6, SLC1A2, IL1R2, RETN | | | | | | | | | |
| OAS1, SIGLEC1, ISG15, HERC6, | 0.861 | 0.851 | 0.870 | 0.912 | 0.858 | 0.967 | 0.965 | 0.927 | 1 |
| SLC1A2, IL1R2, RETN, MMP8 | | | | | | | | | |
| RSAD2, HERC6, SLC1A2, IL1R2, | 0.861 | 0.851 | 0.871 | 0.914 | 0.861 | 0.967 | 0.928 | 0.842 | 1 |
| OLAH, FAM20A, RETN, MMP8 | | | | | | | | | |
| RSAD2, IFI44L, ISG15, SLC1A2, | 0.861 | 0.850 | 0.871 | 0.917 | 0.865 | 0.969 | 0.925 | 0.841 | 1 |
| IL1R2, OLAH, FAM20A, MMP8 | | | | | | | | | |
| RSAD2, IFI27, IFIT1, SIGLEC1, | 0.861 | 0.851 | 0.870 | 0.915 | 0.859 | 0.971 | 0.953 | 0.896 | 1 |
| ISG15, SLC1A2, RETN, MMP8 | | | | | | | | | |
| OAS1, IFI44L, SIGLEC1, HERC6, | 0.861 | 0.850 | 0.871 | 0.903 | 0.840 | 0.966 | 0.964 | 0.926 | 1 |
| IL1R2, OLAH, RETN, MMP8 | | | | | | | | | |
| OAS1, IFIT1, IFI44L, SIGLEC1, | 0.861 | 0.850 | 0.871 | 0.913 | 0.858 | 0.968 | 0.941 | 0.889 | 0.993 |
| ISG15, HERC6, SLC1A2, OLAH | | | | | | | | | |
| RSAD2, OAS1, IFI44L, ISG15, | 0.861 | 0.850 | 0.871 | 0.913 | 0.857 | 0.968 | 0.927 | 0.848 | 1 |
| HERC6, SLC1A2, OLAH, FAM20A | | | | | | | | | |
| RSAD2, IFI27, OAS1, IFI44L, | 0.861 | 0.849 | 0.872 | 0.913 | 0.855 | 0.971 | 0.926 | 0.844 | 1 |
| ISG15, HERC6, SLC1A2, FAM20A | | | | | | | | | |
| RSAD2, SIGLEC1, HERC6, | 0.861 | 0.851 | 0.870 | 0.916 | 0.862 | 0.970 | 0.963 | 0.925 | 1 |
| SLC1A2, IL1R2, OLAH, RETN, MMP8 | | | | | | | | | |
| OAS1, IFIT1, IFI44L, ISG15, | 0.860 | 0.850 | 0.871 | 0.913 | 0.856 | 0.970 | 0.922 | 0.844 | 0.999 |
| SLC1A2, OLAH, FAM20A, MMP8 | | | | | | | | | |
| RSAD2, OAS1, IFIT1, IFI44L, | 0.860 | 0.850 | 0.871 | 0.905 | 0.843 | 0.966 | 0.933 | 0.863 | 1 |
| HERC6, OLAH, FAM20A, MMP8 | | | | | | | | | |
| RSAD2, IFIT1, SIGLEC1, HERC6, | 0.860 | 0.850 | 0.870 | 0.904 | 0.842 | 0.967 | 0.963 | 0.925 | 1 |
| IL1R2, OLAH, RETN, MMP8 | | | | | | | | | |
| RSAD2, OAS1, IFI44L, SIGLEC1, | 0.860 | 0.850 | 0.871 | 0.903 | 0.839 | 0.966 | 0.966 | 0.930 | 1 |
| HERC6, IL1R2, RETN, MMP8 | | | | | | | | | |
| RSAD2, OAS1, SIGLEC1, ISG15, | 0.860 | 0.850 | 0.870 | 0.912 | 0.858 | 0.966 | 0.963 | 0.924 | 1 |
| HERC6, SLC1A2, IL1R2, OLAH | | | | | | | | | |
| OAS1, IFIT1, SIGLEC1, ISG15, | 0.860 | 0.850 | 0.870 | 0.912 | 0.858 | 0.966 | 0.954 | 0.911 | 0.998 |
| HERC6, SLC1A2, IL1R2, OLAH | | | | | | | | | |
| RSAD2, OAS1, IFIT1, IFI44L, | 0.860 | 0.850 | 0.870 | 0.912 | 0.857 | 0.967 | 0.951 | 0.905 | 0.998 |
| SIGLEC1, HERC6, SLC1A2, OLAH | | | | | | | | | |
| RSAD2, IFI44L, ISG15, HERC6, | 0.860 | 0.850 | 0.871 | 0.914 | 0.856 | 0.972 | 0.932 | 0.852 | 1 |
| IL1R2, FAM20A, RETN, MMP8 | | | | | | | | | |
| RSAD2, IFIT1, IFI44L, SIGLEC1, | 0.860 | 0.851 | 0.870 | 0.913 | 0.858 | 0.968 | 0.940 | 0.888 | 0.993 |
| HERC6, SLC1A2, OLAH, MMP8 | | | | | | | | | |
| OAS1, IFIT1, SIGLEC1, HERC6, | 0.860 | 0.850 | 0.871 | 0.916 | 0.863 | 0.969 | 0.943 | 0.893 | 0.994 |
| SLC1A2, IL1R2, OLAH, RETN | | | | | | | | | |
| OAS1, IFIT1, IFI44L, ISG15, | 0.860 | 0.850 | 0.870 | 0.910 | 0.850 | 0.969 | 0.933 | 0.850 | 1 |
| HERC6, IL1R2, FAM20A, MMP8 | | | | | | | | | |
| OAS1, IFI44L, ISG15, HERC6, | 0.860 | 0.850 | 0.871 | 0.906 | 0.846 | 0.967 | 0.939 | 0.866 | 1 |
| IL1R2, OLAH, FAM20A, RETN | | | | | | | | | |
| RSAD2, IFI27, IFI44L, ISG15, | 0.860 | 0.849 | 0.871 | 0.911 | 0.849 | 0.974 | 0.939 | 0.860 | 1 |
| SLC1A2, FAM20A, RETN, MMP8 | | | | | | | | | |
| OAS1, IFIT1, ISG15, HERC6, | 0.860 | 0.850 | 0.871 | 0.912 | 0.858 | 0.967 | 0.928 | 0.844 | 1 |
| SLC1A2, IL1R2, OLAH, FAM20A | | | | | | | | | |
| RSAD2, OAS1, IFIT1, SIGLEC1, | 0.860 | 0.850 | 0.870 | 0.904 | 0.841 | 0.967 | 0.951 | 0.905 | 0.997 |
| HERC6, IL1R2, OLAH, RETN | | | | | | | | | |
| RSAD2, OAS1, IFIT1, IFI44L, | 0.860 | 0.851 | 0.870 | 0.904 | 0.845 | 0.963 | 0.961 | 0.921 | 1 |
| SIGLEC1, HERC6, IL1R2, MMP8 | | | | | | | | | |
| RSAD2, IFIT1, IFI44L, ISG15, | 0.860 | 0.850 | 0.870 | 0.911 | 0.854 | 0.969 | 0.932 | 0.850 | 1 |
| HERC6, IL1R2, FAM20A, MMP8 | | | | | | | | | |
| IFIT1, IFI44L, SIGLEC1, ISG15, | 0.860 | 0.851 | 0.870 | 0.915 | 0.860 | 0.970 | 0.941 | 0.890 | 0.993 |
| HERC6, SLC1A2, OLAH, RETN | | | | | | | | | |
| IFI27, OAS1, IFI44L, ISG15, | 0.860 | 0.849 | 0.871 | 0.911 | 0.854 | 0.968 | 0.927 | 0.845 | 1 |
| HERC6, SLC1A2, FAM20A, MMP8 | | | | | | | | | |
| RSAD2, OAS1, IFI44L, HERC6, | 0.860 | 0.850 | 0.871 | 0.911 | 0.851 | 0.972 | 0.936 | 0.856 | 1 |
| IL1R2, FAM20A, RETN, MMP8 | | | | | | | | | |
| IFI27, OAS1, IFI44L, ISG15, | 0.860 | 0.849 | 0.872 | 0.913 | 0.852 | 0.974 | 0.939 | 0.859 | 1 |
| SLC1A2, FAM20A, RETN, MMP8 | | | | | | | | | |
| OAS1, IFIT1, ISG15, SLC1A2, | 0.860 | 0.851 | 0.870 | 0.917 | 0.865 | 0.969 | 0.936 | 0.858 | 1 |
| IL1R2, OLAH, FAM20A, MMP8 | | | | | | | | | |
| RSAD2, OAS1, IFIT1, SIGLEC1, | 0.860 | 0.851 | 0.870 | 0.912 | 0.857 | 0.967 | 0.957 | 0.911 | 1 |
| HERC6, SLC1A2, IL1R2, RETN | | | | | | | | | |
| RSAD2, OAS1, IFIT1, HERC6, | 0.860 | 0.850 | 0.870 | 0.916 | 0.864 | 0.969 | 0.929 | 0.842 | 1 |
| SLC1A2, IL1R2, FAM20A, MMP8 | | | | | | | | | |
| RSAD2, IFIT1, ISG15, HERC6, | 0.860 | 0.850 | 0.870 | 0.906 | 0.846 | 0.966 | 0.937 | 0.862 | 1 |
| IL1R2, OLAH, FAM20A, RETN | | | | | | | | | |
| RSAD2, IFI27, IFIT1, IFI44L, | 0.860 | 0.851 | 0.870 | 0.912 | 0.853 | 0.971 | 0.930 | 0.860 | 1 |
| HERC6, SLC1A2, RETN, MMP8 | | | | | | | | | |
| OAS1, IFIT1, SIGLEC1, ISG15, | 0.860 | 0.850 | 0.870 | 0.914 | 0.859 | 0.968 | 0.947 | 0.898 | 0.995 |
| HERC6, SLC1A2, OLAH, MMP8 | | | | | | | | | |
| IFI44L, SIGLEC1, ISG15, HERC6, | 0.860 | 0.850 | 0.870 | 0.902 | 0.839 | 0.965 | 0.964 | 0.926 | 1 |
| IL1R2, OLAH, RETN, MMP8 | | | | | | | | | |
| OAS1, IFIT1, IFI44L, SIGLEC1, | 0.860 | 0.851 | 0.869 | 0.907 | 0.849 | 0.964 | 0.962 | 0.922 | 1 |
| HERC6, IL1R2, OLAH, MMP8 | | | | | | | | | |
| RSAD2, IFIT1, SIGLEC1, HERC6, | 0.860 | 0.850 | 0.870 | 0.913 | 0.858 | 0.968 | 0.950 | 0.904 | 0.996 |
| SLC1A2, OLAH, RETN, MMP8 | | | | | | | | | |
| RSAD2, OAS1, IFI44L, SIGLEC1, | 0.860 | 0.850 | 0.870 | 0.908 | 0.851 | 0.966 | 0.937 | 0.860 | 1 |
| HERC6, SLC1A2, FAM20A, MMP8 | | | | | | | | | |
| RSAD2, SIGLEC1, ISG15, HERC6, | 0.860 | 0.850 | 0.870 | 0.907 | 0.846 | 0.969 | 0.961 | 0.921 | 1 |
| IL1R2, OLAH, RETN, MMP8 | | | | | | | | | |
| OAS1, IFIT1, IFI44L, HERC6, | 0.860 | 0.850 | 0.870 | 0.905 | 0.841 | 0.969 | 0.941 | 0.874 | 1 |
| OLAH, FAM20A, RETN, MMP8 | | | | | | | | | |
| RSAD2, OAS1, IFI44L, ISG15, | 0.860 | 0.849 | 0.871 | 0.915 | 0.859 | 0.971 | 0.923 | 0.846 | 1.000 |
| SLC1A2, OLAH, FAM20A, MMP8 | | | | | | | | | |
| OAS1, SIGLEC1, ISG15, HERC6, | 0.860 | 0.849 | 0.871 | 0.914 | 0.860 | 0.968 | 0.957 | 0.912 | 1 |
| SLC1A2, IL1R2, OLAH, RETN | | | | | | | | | |
| OAS1, ISG15, HERC6, SLC1A2, | 0.860 | 0.850 | 0.870 | 0.918 | 0.865 | 0.970 | 0.927 | 0.838 | 1 |
| IL1R2, FAM20A, RETN, MMP8 | | | | | | | | | |
| RSAD2, IFI44L, SIGLEC1, ISG15, | 0.860 | 0.850 | 0.870 | 0.911 | 0.854 | 0.968 | 0.937 | 0.860 | 1 |
| HERC6, SLC1A2, FAM20A, MMP8 | | | | | | | | | |
| RSAD2, IFIT1, HERC6, IL1R2, | 0.860 | 0.850 | 0.870 | 0.908 | 0.846 | 0.969 | 0.945 | 0.873 | 1 |
| OLAH, FAM20A, RETN, MMP8 | | | | | | | | | |
| RSAD2, OAS1, IFI44L, HERC6, | 0.860 | 0.850 | 0.870 | 0.919 | 0.869 | 0.968 | 0.924 | 0.830 | 1 |
| SLC1A2, IL1R2, FAM20A, MMP8 | | | | | | | | | |
| RSAD2, IFI27, OAS1, IFIT1, ISG15, | 0.860 | 0.850 | 0.870 | 0.908 | 0.847 | 0.970 | 0.939 | 0.858 | 1 |
| SLC1A2, FAM20A, RETN | | | | | | | | | |
| RSAD2, IFIT1, SIGLEC1, ISG15, | 0.860 | 0.851 | 0.869 | 0.913 | 0.859 | 0.967 | 0.953 | 0.909 | 0.997 |
| HERC6, SLC1A2, IL1R2, OLAH | | | | | | | | | |
| RSAD2, IFI27, OAS1, IFI44L, | 0.860 | 0.849 | 0.871 | 0.912 | 0.851 | 0.974 | 0.940 | 0.861 | 1 |
| SLC1A2, FAM20A, RETN, MMP8 | | | | | | | | | |
| ISG15, HERC6, SLC1A2, IL1R2, | 0.860 | 0.850 | 0.870 | 0.916 | 0.865 | 0.968 | 0.928 | 0.843 | 1 |
| OLAH, FAM20A, RETN, MMP8 | | | | | | | | | |
| OAS1, HERC6, SLC1A2, IL1R2, | 0.860 | 0.850 | 0.870 | 0.915 | 0.862 | 0.968 | 0.929 | 0.844 | 1 |
| OLAH, FAM20A, RETN, MMP8 | | | | | | | | | |
| RSAD2, OAS1, IFIT1, SIGLEC1, | 0.860 | 0.850 | 0.870 | 0.901 | 0.836 | 0.966 | 0.953 | 0.909 | 0.997 |
| HERC6, OLAH, RETN, MMP8 | | | | | | | | | |
| RSAD2, IFI27, IFIT1, IFI44L, | 0.860 | 0.850 | 0.869 | 0.915 | 0.859 | 0.971 | 0.952 | 0.895 | 1 |
| SIGLEC1, SLC1A2, RETN, MMP8 | | | | | | | | | |
| RSAD2, IFIT1, IFI44L, ISG15, | 0.860 | 0.850 | 0.870 | 0.912 | 0.855 | 0.970 | 0.934 | 0.855 | 1 |
| HERC6, IL1R2, FAM20A, RETN | | | | | | | | | |
| OAS1, ISG15, HERC6, SLC1A2, | 0.860 | 0.849 | 0.871 | 0.913 | 0.858 | 0.968 | 0.927 | 0.842 | 1 |
| IL1R2, OLAH, FAM20A, RETN | | | | | | | | | |
| IFIT1, IFI44L, ISG15, SLC1A2, | 0.860 | 0.850 | 0.870 | 0.913 | 0.858 | 0.968 | 0.926 | 0.846 | 1 |
| IL1R2, OLAH, FAM20A, RETN | | | | | | | | | |
| IFI44L, SIGLEC1, ISG15, SLC1A2, | 0.860 | 0.851 | 0.869 | 0.912 | 0.857 | 0.967 | 0.966 | 0.931 | 1 |
| IL1R2, OLAH, RETN, MMP8 | | | | | | | | | |
| RSAD2, IFI27, OAS1, IFI44L, | 0.860 | 0.851 | 0.869 | 0.907 | 0.850 | 0.963 | 0.913 | 0.838 | 0.988 |
| SIGLEC1, ISG15, HERC6, MMP8 | | | | | | | | | |
| IFI44L, ISG15, HERC6, SLC1A2, | 0.860 | 0.850 | 0.869 | 0.916 | 0.863 | 0.969 | 0.928 | 0.837 | 1 |
| IL1R2, FAM20A, RETN, MMP8 | | | | | | | | | |
| IFIT1, IFI44L, SIGLEC1, ISG15, | 0.860 | 0.850 | 0.869 | 0.906 | 0.845 | 0.968 | 0.950 | 0.904 | 0.996 |
| HERC6, IL1R2, OLAH, RETN | | | | | | | | | |
| IFIT1, IFI44L, SIGLEC1, ISG15, | 0.860 | 0.850 | 0.869 | 0.913 | 0.857 | 0.970 | 0.955 | 0.911 | 1 |
| SLC1A2, OLAH, RETN, MMP8 | | | | | | | | | |
| IFIT1, IFI44L, SIGLEC1, ISG15, | 0.860 | 0.850 | 0.869 | 0.906 | 0.849 | 0.964 | 0.963 | 0.924 | 1 |
| HERC6, IL1R2, OLAH, MMP8 | | | | | | | | | |
| RSAD2, OAS1, IFIT1, SIGLEC1, | 0.860 | 0.850 | 0.870 | 0.912 | 0.857 | 0.967 | 0.951 | 0.905 | 0.998 |
| ISG15, HERC6, SLC1A2, OLAH | | | | | | | | | |
| OAS1, IFIT1, IFI44L, HERC6, | 0.860 | 0.850 | 0.870 | 0.916 | 0.865 | 0.967 | 0.925 | 0.832 | 1 |
| SLC1A2, IL1R2, FAM20A, MMP8 | | | | | | | | | |
| RSAD2, OAS1, SIGLEC1, HERC6, | 0.860 | 0.850 | 0.869 | 0.912 | 0.856 | 0.967 | 0.970 | 0.935 | 1 |
| SLC1A2, IL1R2, RETN, MMP8 | | | | | | | | | |
| SIGLEC1, ISG15, HERC6, SLC1A2, | 0.860 | 0.850 | 0.869 | 0.912 | 0.856 | 0.968 | 0.965 | 0.929 | 1 |
| IL1R2, OLAH, RETN, MMP8 | | | | | | | | | |
| RSAD2, OAS1, IFIT1, IFI44L, | 0.860 | 0.849 | 0.870 | 0.907 | 0.846 | 0.967 | 0.929 | 0.858 | 1 |
| ISG15, HERC6, OLAH, FAM20A | | | | | | | | | |
| RSAD2, OAS1, SIGLEC1, ISG15, | 0.860 | 0.849 | 0.870 | 0.909 | 0.849 | 0.969 | 0.955 | 0.888 | 1 |
| SLC1A2, FAM20A, RETN, MMP8 | | | | | | | | | |
| RSAD2, IFI27, OAS1, IFIT1, | 0.860 | 0.849 | 0.871 | 0.914 | 0.857 | 0.970 | 0.922 | 0.837 | 1 |
| IFI44L, HERC6, SLC1A2, FAM20A | | | | | | | | | |
| RSAD2, IFI44L, HERC6, IL1R2, | 0.860 | 0.849 | 0.870 | 0.914 | 0.854 | 0.973 | 0.941 | 0.868 | 1 |
| OLAH, FAM20A, RETN, MMP8 | | | | | | | | | |
| IFIT1, IFI44L, SIGLEC1, HERC6, | 0.860 | 0.850 | 0.869 | 0.912 | 0.858 | 0.965 | 0.962 | 0.923 | 1 |
| SLC1A2, IL1R2, OLAH, MMP8 | | | | | | | | | |
| RSAD2, OAS1, IFIT1, IFI44L, | 0.860 | 0.850 | 0.869 | 0.903 | 0.842 | 0.964 | 0.957 | 0.914 | 0.999 |
| SIGLEC1, HERC6, IL1R2, OLAH | | | | | | | | | |
| IFIT1, IFI44L, SIGLEC1, ISG15, | 0.860 | 0.851 | 0.868 | 0.912 | 0.858 | 0.965 | 0.966 | 0.931 | 1 |
| SLC1A2, IL1R2, OLAH, MMP8 | | | | | | | | | |
| RSAD2, OAS1, IFI44L, ISG15, | 0.860 | 0.849 | 0.870 | 0.915 | 0.863 | 0.966 | 0.925 | 0.835 | 1 |
| HERC6, SLC1A2, IL1R2, FAM20A | | | | | | | | | |
| RSAD2, OAS1, SIGLEC1, ISG15, | 0.860 | 0.849 | 0.870 | 0.911 | 0.855 | 0.967 | 0.955 | 0.912 | 0.999 |
| HERC6, SLC1A2, OLAH, MMP8 | | | | | | | | | |
| IFIT1, SIGLEC1, ISG15, HERC6, | 0.860 | 0.850 | 0.870 | 0.914 | 0.859 | 0.968 | 0.949 | 0.902 | 0.996 |
| SLC1A2, IL1R2, OLAH, RETN | | | | | | | | | |
| IFIT1, IFI44L, HERC6, SLC1A2, | 0.860 | 0.850 | 0.869 | 0.918 | 0.865 | 0.970 | 0.929 | 0.839 | 1 |
| IL1R2, FAM20A, RETN, MMP8 | | | | | | | | | |
| RSAD2, OAS1, IFIT1, IFI44L, | 0.860 | 0.850 | 0.869 | 0.903 | 0.843 | 0.962 | 0.959 | 0.917 | 1 |
| SIGLEC1, ISG15, HERC6, IL1R2 | | | | | | | | | |
| IFIT1, SIGLEC1, ISG15, HERC6, | 0.860 | 0.850 | 0.869 | 0.912 | 0.858 | 0.966 | 0.965 | 0.929 | 1 |
| SLC1A2, IL1R2, RETN, MMP8 | | | | | | | | | |
| RSAD2, IFIT1, SIGLEC1, HERC6, | 0.860 | 0.850 | 0.869 | 0.911 | 0.857 | 0.966 | 0.961 | 0.922 | 1 |
| SLC1A2, IL1R2, RETN, MMP8 | | | | | | | | | |
| IFI44L, SIGLEC1, HERC6, | 0.860 | 0.850 | 0.869 | 0.913 | 0.858 | 0.968 | 0.967 | 0.933 | 1 |
| SLC1A2, IL1R2, OLAH, RETN, MMP8 | | | | | | | | | |
| OAS1, IFIT1, HERC6, SLC1A2, | 0.860 | 0.850 | 0.869 | 0.915 | 0.860 | 0.969 | 0.930 | 0.845 | 1 |
| IL1R2, FAM20A, RETN, MMP8 | | | | | | | | | |
| RSAD2, IFIT1, IFI44L, SIGLEC1, | 0.859 | 0.850 | 0.869 | 0.904 | 0.842 | 0.966 | 0.955 | 0.912 | 0.999 |
| ISG15, HERC6, IL1R2, RETN | | | | | | | | | |
| OAS1, IFI44L, SIGLEC1, HERC6, | 0.859 | 0.849 | 0.870 | 0.911 | 0.853 | 0.968 | 0.963 | 0.922 | 1 |
| SLC1A2, OLAH, RETN, MMP8 | | | | | | | | | |
| OAS1, IFIT1, IFI44L, SIGLEC1, | 0.859 | 0.850 | 0.869 | 0.903 | 0.844 | 0.961 | 0.961 | 0.920 | 1 |
| ISG15, HERC6, IL1R2, MMP8 | | | | | | | | | |
| OAS1, IFI44L, ISG15, HERC6, | 0.859 | 0.849 | 0.870 | 0.913 | 0.857 | 0.969 | 0.924 | 0.842 | 1 |
| SLC1A2, OLAH, FAM20A, RETN | | | | | | | | | |
| IFI27, OAS1, IFIT1, IFI44L, ISG15, | 0.859 | 0.849 | 0.870 | 0.913 | 0.856 | 0.969 | 0.920 | 0.831 | 1 |
| HERC6, SLC1A2, FAM20A | | | | | | | | | |
| RSAD2, IFIT1, IFI44L, HERC6, | 0.859 | 0.850 | 0.869 | 0.917 | 0.867 | 0.968 | 0.925 | 0.832 | 1 |
| SLC1A2, IL1R2, FAM20A, MMP8 | | | | | | | | | |
| IFI27, OAS1, IFI44L, SIGLEC1, | 0.859 | 0.850 | 0.868 | 0.911 | 0.858 | 0.965 | 0.921 | 0.850 | 0.991 |
| ISG15, HERC6, SLC1A2, MMP8 | | | | | | | | | |
| RSAD2, IFIT1, ISG15, SLC1A2, | 0.859 | 0.850 | 0.869 | 0.914 | 0.862 | 0.967 | 0.935 | 0.856 | 1 |
| IL1R2, OLAH, FAM20A, MMP8 | | | | | | | | | |
| RSAD2, OAS1, ISG15, HERC6, | 0.859 | 0.849 | 0.870 | 0.905 | 0.843 | 0.967 | 0.942 | 0.874 | 1 |
| OLAH, FAM20A, RETN, MMP8 | | | | | | | | | |
| IFIT1, ISG15, HERC6, SLC1A2, | 0.859 | 0.849 | 0.870 | 0.912 | 0.857 | 0.967 | 0.928 | 0.844 | 1 |
| IL1R2, OLAH, FAM20A, RETN | | | | | | | | | |
| RSAD2, IFIT1, IFI44L, HERC6, | 0.859 | 0.849 | 0.870 | 0.918 | 0.865 | 0.970 | 0.913 | 0.826 | 1 |
| SLC1A2, OLAH, FAM20A, RETN | | | | | | | | | |
| RSAD2, OAS1, IFIT1, IL1R2, | 0.859 | 0.849 | 0.869 | 0.907 | 0.845 | 0.969 | 0.947 | 0.878 | 1 |
| OLAH, FAM20A, RETN, MMP8 | | | | | | | | | |
| RSAD2, OAS1, SIGLEC1, ISG15, | 0.859 | 0.849 | 0.869 | 0.910 | 0.855 | 0.965 | 0.961 | 0.916 | 1 |
| HERC6, SLC1A2, IL1R2, RETN | | | | | | | | | |
| IFIT1, ISG15, HERC6, SLC1A2, | 0.859 | 0.850 | 0.869 | 0.915 | 0.860 | 0.969 | 0.930 | 0.844 | 1 |
| IL1R2, FAM20A, RETN, MMP8 | | | | | | | | | |
| RSAD2, OAS1, HERC6, SLC1A2, | 0.859 | 0.849 | 0.870 | 0.912 | 0.858 | 0.967 | 0.926 | 0.840 | 1 |
| IL1R2, OLAH, FAM20A, RETN | | | | | | | | | |
| OAS1, IFIT1, ISG15, HERC6, | 0.859 | 0.849 | 0.870 | 0.911 | 0.856 | 0.966 | 0.929 | 0.843 | 1 |
| SLC1A2, IL1R2, FAM20A, RETN | | | | | | | | | |
| OAS1, IFIT1, IFI44L, SIGLEC1, | 0.859 | 0.850 | 0.869 | 0.905 | 0.843 | 0.967 | 0.947 | 0.899 | 0.995 |
| HERC6, IL1R2, OLAH, RETN | | | | | | | | | |
| RSAD2, OAS1, ISG15, HERC6, | 0.859 | 0.849 | 0.869 | 0.915 | 0.864 | 0.966 | 0.924 | 0.831 | 1 |
| SLC1A2, IL1R2, FAM20A, MMP8 | | | | | | | | | |
| RSAD2, OAS1, ISG15, SLC1A2, | 0.859 | 0.850 | 0.869 | 0.914 | 0.862 | 0.967 | 0.932 | 0.849 | 1 |
| IL1R2, OLAH, FAM20A, MMP8 | | | | | | | | | |
| RSAD2, IFIT1, IFI44L, SIGLEC1, | 0.859 | 0.850 | 0.869 | 0.914 | 0.859 | 0.969 | 0.937 | 0.883 | 0.991 |
| HERC6, SLC1A2, OLAH, RETN | | | | | | | | | |
| RSAD2, IFIT1, IFI44L, ISG15, | 0.859 | 0.850 | 0.869 | 0.908 | 0.848 | 0.968 | 0.934 | 0.865 | 1 |
| HERC6, OLAH, FAM20A, RETN | | | | | | | | | |
| RSAD2, IFIT1, IFI44L, SIGLEC1, | 0.859 | 0.850 | 0.869 | 0.904 | 0.845 | 0.964 | 0.962 | 0.922 | 1 |
| HERC6, IL1R2, OLAH, MMP8 | | | | | | | | | |
| RSAD2, OAS1, SIGLEC1, HERC6, | 0.859 | 0.849 | 0.870 | 0.912 | 0.856 | 0.967 | 0.958 | 0.912 | 1 |
| SLC1A2, IL1R2, OLAH, RETN | | | | | | | | | |
| OAS1, IFI44L, ISG15, HERC6, | 0.859 | 0.849 | 0.870 | 0.917 | 0.867 | 0.967 | 0.924 | 0.830 | 1 |
| SLC1A2, IL1R2, FAM20A, MMP8 | | | | | | | | | |
| OAS1, ISG15, HERC6, SLC1A2, | 0.859 | 0.849 | 0.870 | 0.912 | 0.855 | 0.968 | 0.935 | 0.856 | 1 |
| OLAH, FAM20A, RETN, MMP8 | | | | | | | | | |
| RSAD2, IFIT1, SIGLEC1, HERC6, | 0.859 | 0.849 | 0.869 | 0.915 | 0.861 | 0.970 | 0.951 | 0.905 | 0.997 |
| SLC1A2, IL1R2, OLAH, RETN | | | | | | | | | |
| OAS1, IFIT1, IFI44L, SIGLEC1, | 0.859 | 0.849 | 0.869 | 0.912 | 0.856 | 0.969 | 0.957 | 0.912 | 1 |
| SLC1A2, OLAH, RETN, MMP8 | | | | | | | | | |
| RSAD2, IFIT1, SIGLEC1, ISG15, | 0.859 | 0.849 | 0.869 | 0.912 | 0.857 | 0.967 | 0.947 | 0.898 | 0.996 |
| HERC6, SLC1A2, OLAH, MMP8 | | | | | | | | | |
| OAS1, IFIT1, ISG15, HERC6, | 0.859 | 0.849 | 0.869 | 0.915 | 0.863 | 0.967 | 0.928 | 0.839 | 1 |
| SLC1A2, IL1R2, FAM20A, MMP8 | | | | | | | | | |
| RSAD2, OAS1, IFIT1, IFI44L, | 0.859 | 0.849 | 0.869 | 0.904 | 0.840 | 0.967 | 0.933 | 0.875 | 0.990 |
| SIGLEC1, ISG15, HERC6, RETN | | | | | | | | | |
| RSAD2, IFI44L, SIGLEC1, ISG15, | 0.859 | 0.849 | 0.869 | 0.902 | 0.838 | 0.966 | 0.964 | 0.927 | 1 |
| HERC6, IL1R2, RETN, MMP8 | | | | | | | | | |
| IFIT1, IFI44L, ISG15, HERC6, | 0.859 | 0.849 | 0.869 | 0.916 | 0.866 | 0.967 | 0.924 | 0.830 | 1 |
| SLC1A2, IL1R2, FAM20A, MMP8 | | | | | | | | | |
| RSAD2, IFI44L, SIGLEC1, HERC6, | 0.859 | 0.849 | 0.869 | 0.912 | 0.858 | 0.966 | 0.965 | 0.929 | 1 |
| SLC1A2, IL1R2, OLAH, MMP8 | | | | | | | | | |
| RSAD2, OAS1, IFI44L, SIGLEC1, | 0.859 | 0.849 | 0.869 | 0.897 | 0.824 | 0.969 | 0.960 | 0.915 | 1 |
| ISG15, OLAH, FAM20A, RETN | | | | | | | | | |
| RSAD2, OAS1, IFI44L, SIGLEC1, | 0.859 | 0.850 | 0.869 | 0.904 | 0.846 | 0.963 | 0.962 | 0.922 | 1 |
| HERC6, IL1R2, OLAH, MMP8 | | | | | | | | | |
| RSAD2, OAS1, IFI44L, SIGLEC1, | 0.859 | 0.849 | 0.869 | 0.902 | 0.837 | 0.967 | 0.940 | 0.869 | 1 |
| ISG15, HERC6, FAM20A, MMP8 | | | | | | | | | |
| IFI27, OAS1, IFIT1, ISG15, HERC6, | 0.859 | 0.848 | 0.870 | 0.915 | 0.860 | 0.969 | 0.925 | 0.840 | 1 |
| SLC1A2, FAM20A, MMP8 | | | | | | | | | |
| RSAD2, ISG15, HERC6, SLC1A2, | 0.859 | 0.849 | 0.869 | 0.916 | 0.864 | 0.969 | 0.928 | 0.839 | 1 |
| IL1R2, FAM20A, RETN, MMP8 | | | | | | | | | |
| OAS1, IFIT1, IFI44L, HERC6, | 0.859 | 0.850 | 0.869 | 0.905 | 0.844 | 0.967 | 0.934 | 0.857 | 1 |
| IL1R2, OLAH, FAM20A, RETN | | | | | | | | | |
| IFIT1, IFI44L, SIGLEC1, HERC6, | 0.859 | 0.850 | 0.868 | 0.911 | 0.856 | 0.965 | 0.964 | 0.927 | 1 |
| SLC1A2, IL1R2, RETN, MMP8 | | | | | | | | | |
| RSAD2, IFI44L, HERC6, SLC1A2, | 0.859 | 0.848 | 0.870 | 0.915 | 0.861 | 0.968 | 0.924 | 0.840 | 1 |
| IL1R2, OLAH, FAM20A, RETN | | | | | | | | | |
| OAS1, IFI44L, SIGLEC1, HERC6, | 0.859 | 0.849 | 0.869 | 0.912 | 0.859 | 0.966 | 0.961 | 0.922 | 1 |
| SLC1A2, IL1R2, OLAH, MMP8 | | | | | | | | | |
| OAS1, IFIT1, ISG15, HERC6, | 0.859 | 0.849 | 0.869 | 0.905 | 0.842 | 0.967 | 0.947 | 0.882 | 1 |
| OLAH, FAM20A, RETN, MMP8 | | | | | | | | | |
| RSAD2, OAS1, IFIT1, SIGLEC1, | 0.859 | 0.849 | 0.869 | 0.906 | 0.848 | 0.963 | 0.960 | 0.919 | 1 |
| HERC6, IL1R2, OLAH, MMP8 | | | | | | | | | |
| RSAD2, ISG15, HERC6, SLC1A2, | 0.859 | 0.849 | 0.869 | 0.913 | 0.858 | 0.968 | 0.927 | 0.842 | 1 |
| IL1R2, OLAH, FAM20A, RETN | | | | | | | | | |
| RSAD2, OAS1, SIGLEC1, ISG15, | 0.859 | 0.849 | 0.869 | 0.904 | 0.847 | 0.962 | 0.961 | 0.920 | 1 |
| HERC6, IL1R2, OLAH, MMP8 | | | | | | | | | |
| OAS1, IFIT1, IFI44L, ISG15, | 0.859 | 0.849 | 0.869 | 0.915 | 0.865 | 0.966 | 0.926 | 0.835 | 1 |
| HERC6, SLC1A2, IL1R2, FAM20A | | | | | | | | | |
| OAS1, IFIT1, SIGLEC1, ISG15, | 0.859 | 0.850 | 0.868 | 0.913 | 0.859 | 0.966 | 0.965 | 0.929 | 1 |
| SLC1A2, IL1R2, OLAH, MMP8 | | | | | | | | | |
| IFIT1, SIGLEC1, ISG15, SLC1A2, | 0.859 | 0.850 | 0.868 | 0.913 | 0.858 | 0.967 | 0.966 | 0.931 | 1 |
| IL1R2, OLAH, RETN, MMP8 | | | | | | | | | |
| RSAD2, IFIT1, IFI44L, ISG15, | 0.859 | 0.849 | 0.869 | 0.915 | 0.864 | 0.966 | 0.923 | 0.830 | 1 |
| HERC6, SLC1A2, IL1R2, FAM20A | | | | | | | | | |
| IFI27, OAS1, IFIT1, ISG15, | 0.859 | 0.848 | 0.870 | 0.909 | 0.846 | 0.972 | 0.941 | 0.861 | 1 |
| SLC1A2, FAM20A, RETN, MMP8 | | | | | | | | | |
| OAS1, IFI44L, SIGLEC1, HERC6, | 0.859 | 0.849 | 0.868 | 0.911 | 0.856 | 0.966 | 0.965 | 0.928 | 1 |
| SLC1A2, IL1R2, RETN, MMP8 | | | | | | | | | |
| RSAD2, OAS1, IFIT1, HERC6, | 0.859 | 0.849 | 0.869 | 0.909 | 0.853 | 0.966 | 0.929 | 0.849 | 1 |
| SLC1A2, OLAH, FAM20A, MMP8 | | | | | | | | | |
| IFI44L, SIGLEC1, ISG15, HERC6, | 0.859 | 0.849 | 0.868 | 0.913 | 0.859 | 0.966 | 0.964 | 0.927 | 1 |
| SLC1A2, IL1R2, OLAH, MMP8 | | | | | | | | | |
| OAS1, IFIT1, IFI44L, SIGLEC1, | 0.859 | 0.849 | 0.869 | 0.913 | 0.857 | 0.968 | 0.950 | 0.903 | 0.997 |
| ISG15, SLC1A2, OLAH, MMP8 | | | | | | | | | |
| RSAD2, OAS1, IFIT1, SIGLEC1, | 0.859 | 0.849 | 0.869 | 0.905 | 0.842 | 0.967 | 0.948 | 0.899 | 0.997 |
| ISG15, HERC6, OLAH, RETN | | | | | | | | | |
| RSAD2, OAS1, IFIT1, HERC6, | 0.859 | 0.849 | 0.869 | 0.912 | 0.858 | 0.966 | 0.928 | 0.844 | 1 |
| SLC1A2, IL1R2, OLAH, FAM20A | | | | | | | | | |
| RSAD2, OAS1, IFI44L, SIGLEC1, | 0.859 | 0.849 | 0.869 | 0.902 | 0.839 | 0.964 | 0.957 | 0.913 | 1 |
| ISG15, HERC6, IL1R2, RETN | | | | | | | | | |
| RSAD2, OAS1, IFI44L, HERC6, | 0.859 | 0.848 | 0.869 | 0.907 | 0.847 | 0.968 | 0.935 | 0.860 | 1 |
| IL1R2, OLAH, FAM20A, RETN | | | | | | | | | |
| RSAD2, IFI27, IFI44L, SIGLEC1, | 0.859 | 0.849 | 0.868 | 0.915 | 0.859 | 0.971 | 0.950 | 0.891 | 1 |
| ISG15, SLC1A2, RETN, MMP8 | | | | | | | | | |
| OAS1, IFIT1, IFI44L, SIGLEC1, | 0.859 | 0.849 | 0.868 | 0.913 | 0.859 | 0.966 | 0.948 | 0.900 | 0.996 |
| HERC6, SLC1A2, IL1R2, RETN | | | | | | | | | |
| RSAD2, OAS1, IFIT1, HERC6, | 0.859 | 0.849 | 0.869 | 0.906 | 0.844 | 0.967 | 0.937 | 0.863 | 1 |
| IL1R2, OLAH, FAM20A, RETN | | | | | | | | | |
| RSAD2, IFIT1, ISG15, HERC6, | 0.859 | 0.849 | 0.869 | 0.912 | 0.858 | 0.966 | 0.928 | 0.844 | 1 |
| SLC1A2, IL1R2, OLAH, FAM20A | | | | | | | | | |
| RSAD2, OAS1, IFIT1, IFI44L, | 0.859 | 0.849 | 0.868 | 0.905 | 0.843 | 0.968 | 0.933 | 0.863 | 1 |
| HERC6, OLAH, FAM20A, RETN | | | | | | | | | |
| OAS1, IFIT1, SLC1A2, IL1R2, | 0.859 | 0.849 | 0.869 | 0.913 | 0.858 | 0.967 | 0.938 | 0.862 | 1 |
| OLAH, FAM20A, RETN, MMP8 | | | | | | | | | |
| RSAD2, OAS1, ISG15, IL1R2, | 0.859 | 0.849 | 0.869 | 0.907 | 0.845 | 0.968 | 0.938 | 0.861 | 1 |
| OLAH, FAM20A, RETN, MMP8 | | | | | | | | | |
| RSAD2, IFI44L, SLC1A2, IL1R2, | 0.859 | 0.849 | 0.869 | 0.918 | 0.865 | 0.970 | 0.934 | 0.858 | 1 |
| OLAH, FAM20A, RETN, MMP8 | | | | | | | | | |
| RSAD2, OAS1, IFIT1, IFI44L, | 0.859 | 0.849 | 0.868 | 0.902 | 0.838 | 0.966 | 0.939 | 0.886 | 0.992 |
| SIGLEC1, HERC6, OLAH, RETN | | | | | | | | | |
| RSAD2, OAS1, IFI44L, SIGLEC1, | 0.859 | 0.848 | 0.869 | 0.911 | 0.855 | 0.968 | 0.942 | 0.870 | 1 |
| HERC6, SLC1A2, FAM20A, RETN | | | | | | | | | |
| IFIT1, IFI44L, SIGLEC1, ISG15, | 0.859 | 0.849 | 0.868 | 0.913 | 0.859 | 0.967 | 0.953 | 0.909 | 0.997 |
| HERC6, SLC1A2, IL1R2, OLAH | | | | | | | | | |
| RSAD2, IFIT1, IFI44L, SIGLEC1, | 0.859 | 0.850 | 0.868 | 0.903 | 0.844 | 0.963 | 0.959 | 0.917 | 1 |
| ISG15, HERC6, IL1R2, MMP8 | | | | | | | | | |
| OAS1, IFIT1, IFI44L, SIGLEC1, | 0.859 | 0.849 | 0.868 | 0.907 | 0.849 | 0.965 | 0.957 | 0.914 | 0.999 |
| ISG15, HERC6, IL1R2, OLAH | | | | | | | | | |
| IFIT1, ISG15, HERC6, SLC1A2, | 0.859 | 0.848 | 0.869 | 0.909 | 0.851 | 0.967 | 0.934 | 0.855 | 1 |
| OLAH, FAM20A, RETN, MMP8 | | | | | | | | | |
| OAS1, IFIT1, IFI44L, SIGLEC1, | 0.858 | 0.848 | 0.869 | 0.913 | 0.859 | 0.967 | 0.952 | 0.907 | 0.997 |
| HERC6, SLC1A2, IL1R2, OLAH | | | | | | | | | |
| RSAD2, IFIT1, SIGLEC1, ISG15, | 0.858 | 0.849 | 0.868 | 0.905 | 0.843 | 0.967 | 0.950 | 0.904 | 0.996 |
| HERC6, IL1R2, OLAH, RETN | | | | | | | | | |
| RSAD2, OAS1, IFIT1, ISG15, | 0.858 | 0.849 | 0.868 | 0.911 | 0.857 | 0.965 | 0.928 | 0.842 | 1 |
| HERC6, SLC1A2, IL1R2, FAM20A | | | | | | | | | |
| OAS1, IFIT1, SIGLEC1, ISG15, | 0.858 | 0.849 | 0.868 | 0.905 | 0.847 | 0.962 | 0.961 | 0.921 | 1 |
| HERC6, IL1R2, OLAH, MMP8 | | | | | | | | | |
| OAS1, IFIT1, SIGLEC1, SLC1A2, | 0.858 | 0.849 | 0.868 | 0.914 | 0.860 | 0.967 | 0.966 | 0.931 | 1 |
| IL1R2, OLAH, RETN, MMP8 | | | | | | | | | |
| OAS1, IFIT1, IFI44L, ISG15, IL1R2, | 0.858 | 0.849 | 0.868 | 0.907 | 0.845 | 0.968 | 0.945 | 0.874 | 1 |
| OLAH, FAM20A, MMP8 | | | | | | | | | |
| RSAD2, OAS1, IFIT1, SIGLEC1, | 0.858 | 0.849 | 0.868 | 0.911 | 0.855 | 0.967 | 0.952 | 0.907 | 0.998 |
| ISG15, SLC1A2, OLAH, MMP8 | | | | | | | | | |
| RSAD2, IFIT1, ISG15, HERC6, | 0.858 | 0.849 | 0.868 | 0.915 | 0.863 | 0.967 | 0.929 | 0.842 | 1 |
| SLC1A2, IL1R2, FAM20A, MMP8 | | | | | | | | | |
| RSAD2, OAS1, IFIT1, SIGLEC1, | 0.858 | 0.849 | 0.868 | 0.907 | 0.849 | 0.964 | 0.954 | 0.910 | 0.998 |
| ISG15, HERC6, IL1R2, OLAH | | | | | | | | | |
| RSAD2, OAS1, SIGLEC1, SLC1A2, | 0.858 | 0.848 | 0.868 | 0.914 | 0.859 | 0.968 | 0.972 | 0.940 | 1 |
| IL1R2, OLAH, RETN, MMP8 | | | | | | | | | |
| OAS1, IFIT1, SIGLEC1, ISG15, | 0.858 | 0.849 | 0.868 | 0.913 | 0.858 | 0.968 | 0.957 | 0.913 | 1.000 |
| SLC1A2, IL1R2, OLAH, RETN | | | | | | | | | |
| RSAD2, IFIT1, SIGLEC1, ISG15, | 0.858 | 0.848 | 0.868 | 0.904 | 0.841 | 0.968 | 0.959 | 0.918 | 0.999 |
| HERC6, OLAH, RETN, MMP8 | | | | | | | | | |
| RSAD2, IFIT1, SIGLEC1, ISG15, | 0.858 | 0.849 | 0.867 | 0.913 | 0.859 | 0.966 | 0.966 | 0.931 | 1 |
| SLC1A2, IL1R2, OLAH, MMP8 | | | | | | | | | |
| RSAD2, OAS1, IFI44L, SIGLEC1, | 0.858 | 0.849 | 0.868 | 0.903 | 0.843 | 0.963 | 0.958 | 0.915 | 1.000 |
| ISG15, HERC6, IL1R2, OLAH | | | | | | | | | |
| RSAD2, OAS1, IFIT1, IFI44L, | 0.858 | 0.849 | 0.868 | 0.910 | 0.854 | 0.966 | 0.954 | 0.910 | 0.999 |
| SIGLEC1, SLC1A2, OLAH, MMP8 | | | | | | | | | |
| RSAD2, OAS1, ISG15, HERC6, | 0.858 | 0.848 | 0.869 | 0.912 | 0.858 | 0.966 | 0.925 | 0.836 | 1 |
| SLC1A2, IL1R2, FAM20A, RETN | | | | | | | | | |
| OAS1, IFIT1, SIGLEC1, ISG15, | 0.858 | 0.848 | 0.868 | 0.911 | 0.855 | 0.968 | 0.957 | 0.914 | 0.999 |
| SLC1A2, OLAH, RETN, MMP8 | | | | | | | | | |
| RSAD2, IFIT1, HERC6, SLC1A2, | 0.858 | 0.849 | 0.868 | 0.915 | 0.860 | 0.969 | 0.930 | 0.845 | 1 |
| IL1R2, FAM20A, RETN, MMP8 | | | | | | | | | |
| RSAD2, IFI27, IFIT1, ISG15, | 0.858 | 0.848 | 0.869 | 0.908 | 0.845 | 0.971 | 0.942 | 0.864 | 1 |
| SLC1A2, FAM20A, RETN, MMP8 | | | | | | | | | |
| OAS1, SIGLEC1, ISG15, SLC1A2, | 0.858 | 0.849 | 0.868 | 0.913 | 0.859 | 0.967 | 0.966 | 0.931 | 1 |
| IL1R2, OLAH, RETN, MMP8 | | | | | | | | | |
| RSAD2, IFIT1, IFI44L, SIGLEC1, | 0.858 | 0.849 | 0.868 | 0.913 | 0.859 | 0.967 | 0.954 | 0.911 | 0.998 |
| HERC6, SLC1A2, IL1R2, OLAH | | | | | | | | | |
| RSAD2, IFI44L, SIGLEC1, ISG15, | 0.858 | 0.848 | 0.868 | 0.910 | 0.849 | 0.970 | 0.950 | 0.883 | 1 |
| SLC1A2, FAM20A, RETN, MMP8 | | | | | | | | | |
| RSAD2, OAS1, SIGLEC1, ISG15, | 0.858 | 0.848 | 0.869 | 0.905 | 0.844 | 0.966 | 0.958 | 0.915 | 1 |
| HERC6, IL1R2, OLAH, RETN | | | | | | | | | |
| RSAD2, IFI27, OAS1, IFIT1, | 0.858 | 0.848 | 0.869 | 0.909 | 0.846 | 0.972 | 0.943 | 0.866 | 1 |
| SLC1A2, FAM20A, RETN, MMP8 | | | | | | | | | |
| RSAD2, IFIT1, IFI44L, ISG15, | 0.858 | 0.848 | 0.869 | 0.905 | 0.844 | 0.966 | 0.933 | 0.862 | 1 |
| HERC6, OLAH, FAM20A, MMP8 | | | | | | | | | |
| RSAD2, IFIT1, SIGLEC1, SLC1A2, | 0.858 | 0.849 | 0.868 | 0.914 | 0.859 | 0.968 | 0.970 | 0.937 | 1 |
| IL1R2, OLAH, RETN, MMP8 | | | | | | | | | |
| RSAD2, OAS1, IFI44L, SIGLEC1, | 0.858 | 0.848 | 0.869 | 0.904 | 0.836 | 0.971 | 0.953 | 0.889 | 1 |
| ISG15, FAM20A, RETN, MMP8 | | | | | | | | | |
| RSAD2, OAS1, IFI44L, SIGLEC1, | 0.858 | 0.848 | 0.869 | 0.910 | 0.851 | 0.970 | 0.949 | 0.878 | 1 |
| SLC1A2, FAM20A, RETN, MMP8 | | | | | | | | | |
| IFIT1, IFI44L, SIGLEC1, ISG15, | 0.858 | 0.849 | 0.867 | 0.909 | 0.854 | 0.964 | 0.955 | 0.912 | 0.998 |
| HERC6, SLC1A2, IL1R2, RETN | | | | | | | | | |
| RSAD2, ISG15, HERC6, SLC1A2, | 0.858 | 0.848 | 0.868 | 0.911 | 0.854 | 0.968 | 0.935 | 0.856 | 1 |
| OLAH, FAM20A, RETN, MMP8 | | | | | | | | | |
| RSAD2, OAS1, IFIT1, SIGLEC1, | 0.858 | 0.849 | 0.868 | 0.911 | 0.856 | 0.965 | 0.968 | 0.934 | 1 |
| SLC1A2, IL1R2, OLAH, MMP8 | | | | | | | | | |
| RSAD2, IFI44L, SIGLEC1, HERC6, | 0.858 | 0.848 | 0.868 | 0.902 | 0.838 | 0.966 | 0.963 | 0.924 | 1 |
| IL1R2, OLAH, RETN, MMP8 | | | | | | | | | |
| IFIT1, IFI44L, SIGLEC1, SLC1A2, | 0.858 | 0.849 | 0.868 | 0.914 | 0.860 | 0.968 | 0.967 | 0.933 | 1 |
| IL1R2, OLAH, RETN, MMP8 | | | | | | | | | |
| OAS1, IFIT1, ISG15, HERC6, | 0.858 | 0.848 | 0.869 | 0.911 | 0.855 | 0.968 | 0.934 | 0.855 | 1 |
| SLC1A2, OLAH, FAM20A, RETN | | | | | | | | | |
| RSAD2, OAS1, IFIT1, IFI44L, | 0.858 | 0.848 | 0.868 | 0.906 | 0.846 | 0.967 | 0.923 | 0.843 | 1 |
| ISG15, HERC6, FAM20A, RETN | | | | | | | | | |
| RSAD2, OAS1, IFIT1, SIGLEC1, | 0.858 | 0.849 | 0.868 | 0.908 | 0.855 | 0.962 | 0.966 | 0.929 | 1 |
| ISG15, HERC6, SLC1A2, IL1R2 | | | | | | | | | |
| IFIT1, ISG15, SLC1A2, IL1R2, | 0.858 | 0.848 | 0.868 | 0.914 | 0.861 | 0.967 | 0.938 | 0.864 | 1 |
| OLAH, FAM20A, RETN, MMP8 | | | | | | | | | |
| IFIT1, IFI44L, SIGLEC1, HERC6, | 0.858 | 0.848 | 0.868 | 0.915 | 0.862 | 0.969 | 0.949 | 0.902 | 0.996 |
| SLC1A2, IL1R2, OLAH, RETN | | | | | | | | | |
| RSAD2, SIGLEC1, ISG15, HERC6, | 0.858 | 0.848 | 0.868 | 0.915 | 0.861 | 0.969 | 0.958 | 0.916 | 0.999 |
| SLC1A2, IL1R2, OLAH, RETN | | | | | | | | | |
| RSAD2, SIGLEC1, ISG15, SLC1A2, | 0.858 | 0.849 | 0.868 | 0.912 | 0.857 | 0.967 | 0.968 | 0.935 | 1 |
| IL1R2, OLAH, RETN, MMP8 | | | | | | | | | |
| RSAD2, IFIT1, IFI44L, SIGLEC1, | 0.858 | 0.849 | 0.868 | 0.912 | 0.857 | 0.967 | 0.940 | 0.888 | 0.993 |
| ISG15, HERC6, SLC1A2, OLAH | | | | | | | | | |
| RSAD2, SIGLEC1, ISG15, HERC6, | 0.858 | 0.848 | 0.868 | 0.911 | 0.854 | 0.968 | 0.951 | 0.906 | 0.997 |
| SLC1A2, OLAH, RETN, MMP8 | | | | | | | | | |
| RSAD2, IFI27, OAS1, IFIT1, | 0.858 | 0.847 | 0.869 | 0.900 | 0.834 | 0.967 | 0.933 | 0.854 | 1 |
| IFI44L, ISG15, FAM20A, MMP8 | | | | | | | | | |
| RSAD2, IFIT1, IFI44L, SIGLEC1, | 0.858 | 0.849 | 0.867 | 0.903 | 0.840 | 0.967 | 0.949 | 0.902 | 0.996 |
| HERC6, IL1R2, OLAH, RETN | | | | | | | | | |
| RSAD2, OAS1, IFIT1, IFI44L, | 0.858 | 0.848 | 0.868 | 0.908 | 0.847 | 0.969 | 0.934 | 0.853 | 1 |
| HERC6, IL1R2, FAM20A, RETN | | | | | | | | | |
| RSAD2, OAS1, ISG15, HERC6, | 0.858 | 0.848 | 0.868 | 0.912 | 0.857 | 0.967 | 0.932 | 0.852 | 1 |
| SLC1A2, OLAH, FAM20A, MMP8 | | | | | | | | | |
| IFIT1, IFI44L, ISG15, HERC6, | 0.858 | 0.848 | 0.868 | 0.915 | 0.862 | 0.968 | 0.922 | 0.829 | 1 |
| SLC1A2, IL1R2, FAM20A, RETN | | | | | | | | | |
| RSAD2, IFIT1, SIGLEC1, ISG15, | 0.858 | 0.848 | 0.868 | 0.912 | 0.856 | 0.968 | 0.945 | 0.895 | 0.994 |
| HERC6, SLC1A2, OLAH, RETN | | | | | | | | | |
| OAS1, IFIT1, IFI44L, SIGLEC1, | 0.858 | 0.848 | 0.868 | 0.904 | 0.841 | 0.967 | 0.933 | 0.875 | 0.990 |
| ISG15, HERC6, RETN, MMP8 | | | | | | | | | |
| OAS1, IFI44L, HERC6, SLC1A2, | 0.858 | 0.847 | 0.869 | 0.913 | 0.858 | 0.967 | 0.923 | 0.838 | 1 |
| IL1R2, OLAH, FAM20A, RETN | | | | | | | | | |
| RSAD2, OAS1, IFIT1, IFI44L, | 0.858 | 0.848 | 0.868 | 0.914 | 0.862 | 0.965 | 0.924 | 0.833 | 1 |
| HERC6, SLC1A2, IL1R2, FAM20A | | | | | | | | | |
| OAS1, IFI44L, SIGLEC1, ISG15, | 0.858 | 0.847 | 0.869 | 0.908 | 0.851 | 0.966 | 0.942 | 0.868 | 1 |
| HERC6, SLC1A2, FAM20A, MMP8 | | | | | | | | | |
| OAS1, IFI44L, SIGLEC1, ISG15, | 0.858 | 0.848 | 0.868 | 0.910 | 0.854 | 0.965 | 0.958 | 0.912 | 1 |
| HERC6, SLC1A2, IL1R2, RETN | | | | | | | | | |
| RSAD2, OAS1, SIGLEC1, ISG15, | 0.858 | 0.848 | 0.868 | 0.911 | 0.857 | 0.966 | 0.967 | 0.933 | 1 |
| SLC1A2, IL1R2, OLAH, MMP8 | | | | | | | | | |
| RSAD2, OAS1, IFI44L, HERC6, | 0.858 | 0.847 | 0.869 | 0.914 | 0.859 | 0.969 | 0.923 | 0.842 | 1 |
| SLC1A2, OLAH, FAM20A, RETN | | | | | | | | | |
| RSAD2, IFI27, OAS1, ISG15, | 0.858 | 0.847 | 0.869 | 0.915 | 0.860 | 0.969 | 0.924 | 0.839 | 1 |
| HERC6, SLC1A2, FAM20A, MMP8 | | | | | | | | | |
| RSAD2, OAS1, SIGLEC1, ISG15, | 0.858 | 0.848 | 0.868 | 0.912 | 0.856 | 0.969 | 0.953 | 0.904 | 1 |
| HERC6, SLC1A2, OLAH, RETN | | | | | | | | | |
| OAS1, IFIT1, HERC6, SLC1A2, | 0.858 | 0.848 | 0.868 | 0.909 | 0.851 | 0.967 | 0.932 | 0.852 | 1 |
| OLAH, FAM20A, RETN, MMP8 | | | | | | | | | |
| OAS1, IFI44L, HERC6, SLC1A2, | 0.858 | 0.848 | 0.868 | 0.915 | 0.862 | 0.969 | 0.928 | 0.838 | 1 |
| IL1R2, FAM20A, RETN, MMP8 | | | | | | | | | |
| OAS1, IFIT1, IFI44L, SIGLEC1, | 0.858 | 0.848 | 0.868 | 0.903 | 0.841 | 0.965 | 0.963 | 0.925 | 1 |
| IL1R2, OLAH, RETN, MMP8 | | | | | | | | | |
| RSAD2, IFIT1, IFI44L, SIGLEC1, | 0.858 | 0.849 | 0.867 | 0.913 | 0.859 | 0.967 | 0.968 | 0.935 | 1 |
| SLC1A2, IL1R2, OLAH, MMP8 | | | | | | | | | |
| RSAD2, OAS1, IFI44L, HERC6, | 0.858 | 0.848 | 0.868 | 0.908 | 0.846 | 0.970 | 0.937 | 0.868 | 1 |
| OLAH, FAM20A, RETN, MMP8 | | | | | | | | | |
| RSAD2, IFI44L, ISG15, HERC6, | 0.858 | 0.847 | 0.868 | 0.917 | 0.867 | 0.967 | 0.924 | 0.830 | 1 |
| SLC1A2, IL1R2, FAM20A, MMP8 | | | | | | | | | |
| RSAD2, IFIT1, IFI44L, HERC6, | 0.858 | 0.848 | 0.868 | 0.909 | 0.847 | 0.970 | 0.939 | 0.859 | 1 |
| IL1R2, FAM20A, RETN, MMP8 | | | | | | | | | |
| OAS1, IFIT1, IFI44L, SIGLEC1, | 0.858 | 0.848 | 0.868 | 0.915 | 0.860 | 0.971 | 0.940 | 0.885 | 0.995 |
| ISG15, SLC1A2, OLAH, RETN | | | | | | | | | |
| RSAD2, OAS1, IFI44L, ISG15, | 0.858 | 0.847 | 0.869 | 0.913 | 0.858 | 0.969 | 0.928 | 0.848 | 1 |
| SLC1A2, IL1R2, OLAH, FAM20A | | | | | | | | | |
| RSAD2, OAS1, IFIT1, SIGLEC1, | 0.858 | 0.848 | 0.868 | 0.913 | 0.858 | 0.968 | 0.967 | 0.931 | 1 |
| ISG15, SLC1A2, IL1R2, OLAH | | | | | | | | | |
| RSAD2, OAS1, IFIT1, IFI44L, | 0.858 | 0.848 | 0.868 | 0.916 | 0.864 | 0.967 | 0.930 | 0.844 | 1 |
| SLC1A2, IL1R2, FAM20A, MMP8 | | | | | | | | | |
| RSAD2, IFI44L, ISG15, HERC6, | 0.858 | 0.847 | 0.869 | 0.914 | 0.858 | 0.970 | 0.924 | 0.843 | 1 |
| SLC1A2, OLAH, FAM20A, RETN | | | | | | | | | |
| RSAD2, IFI27, IFIT1, IFI44L, | 0.858 | 0.849 | 0.867 | 0.907 | 0.850 | 0.963 | 0.907 | 0.832 | 0.981 |
| SIGLEC1, ISG15, HERC6, MMP8 | | | | | | | | | |
| OAS1, IFI44L, ISG15, HERC6, | 0.858 | 0.847 | 0.869 | 0.916 | 0.865 | 0.967 | 0.926 | 0.837 | 1 |
| SLC1A2, IL1R2, FAM20A, RETN | | | | | | | | | |
| OAS1, IFIT1, IFI44L, SIGLEC1, | 0.858 | 0.848 | 0.867 | 0.909 | 0.856 | 0.962 | 0.963 | 0.924 | 1 |
| HERC6, SLC1A2, IL1R2, MMP8 | | | | | | | | | |
| OAS1, IFIT1, IFI44L, SIGLEC1, | 0.858 | 0.848 | 0.867 | 0.912 | 0.858 | 0.966 | 0.966 | 0.931 | 1 |
| SLC1A2, IL1R2, OLAH, MMP8 | | | | | | | | | |
| RSAD2, OAS1, IFIT1, IFI44L, | 0.858 | 0.848 | 0.868 | 0.914 | 0.859 | 0.969 | 0.928 | 0.848 | 1 |
| SLC1A2, IL1R2, OLAH, FAM20A | | | | | | | | | |
| RSAD2, IFIT1, IFI44L, SIGLEC1, | 0.858 | 0.848 | 0.868 | 0.902 | 0.837 | 0.967 | 0.953 | 0.910 | 0.997 |
| HERC6, OLAH, RETN, MMP8 | | | | | | | | | |
| OAS1, IFI44L, SIGLEC1, ISG15, | 0.858 | 0.847 | 0.868 | 0.913 | 0.857 | 0.970 | 0.951 | 0.901 | 1 |
| HERC6, SLC1A2, OLAH, RETN | | | | | | | | | |
| RSAD2, IFIT1, SIGLEC1, ISG15, | 0.858 | 0.849 | 0.867 | 0.909 | 0.854 | 0.965 | 0.957 | 0.914 | 0.999 |
| HERC6, SLC1A2, IL1R2, RETN | | | | | | | | | |
| RSAD2, OAS1, IFIT1, SIGLEC1, | 0.858 | 0.848 | 0.867 | 0.913 | 0.857 | 0.968 | 0.966 | 0.928 | 1 |
| SLC1A2, IL1R2, OLAH, RETN | | | | | | | | | |
| RSAD2, OAS1, HERC6, SLC1A2, | 0.858 | 0.848 | 0.868 | 0.910 | 0.853 | 0.967 | 0.934 | 0.854 | 1 |
| OLAH, FAM20A, RETN, MMP8 | | | | | | | | | |
| OAS1, IFI44L, SIGLEC1, ISG15, | 0.858 | 0.848 | 0.867 | 0.903 | 0.844 | 0.962 | 0.963 | 0.924 | 1 |
| HERC6, IL1R2, OLAH, MMP8 | | | | | | | | | |
| RSAD2, OAS1, ISG15, HERC6, | 0.858 | 0.847 | 0.868 | 0.913 | 0.857 | 0.968 | 0.932 | 0.851 | 1 |
| SLC1A2, OLAH, FAM20A, RETN | | | | | | | | | |
| RSAD2, IFIT1, IFI44L, HERC6, | 0.858 | 0.848 | 0.868 | 0.908 | 0.846 | 0.970 | 0.936 | 0.867 | 1 |
| OLAH, FAM20A, RETN, MMP8 | | | | | | | | | |
| OAS1, IFIT1, ISG15, IL1R2, OLAH, | 0.858 | 0.848 | 0.867 | 0.905 | 0.842 | 0.969 | 0.945 | 0.873 | 1 |
| FAM20A, RETN, MMP8 | | | | | | | | | |
| IFI44L, SIGLEC1, ISG15, HERC6, | 0.858 | 0.848 | 0.868 | 0.910 | 0.852 | 0.968 | 0.961 | 0.922 | 1 |
| SLC1A2, OLAH, RETN, MMP8 | | | | | | | | | |
| RSAD2, IFIT1, IFI44L, SLC1A2, | 0.858 | 0.848 | 0.867 | 0.913 | 0.855 | 0.970 | 0.927 | 0.854 | 1 |
| OLAH, FAM20A, RETN, MMP8 | | | | | | | | | |
| RSAD2, OAS1, IFIT1, SIGLEC1, | 0.858 | 0.848 | 0.867 | 0.905 | 0.848 | 0.962 | 0.960 | 0.917 | 1 |
| ISG15, HERC6, IL1R2, MMP8 | | | | | | | | | |
| IFI44L, SIGLEC1, ISG15, HERC6, | 0.858 | 0.848 | 0.868 | 0.914 | 0.859 | 0.969 | 0.958 | 0.916 | 1.000 |
| SLC1A2, IL1R2, OLAH, RETN | | | | | | | | | |
| OAS1, IFI44L, SIGLEC1, ISG15, | 0.858 | 0.847 | 0.868 | 0.902 | 0.840 | 0.964 | 0.957 | 0.913 | 1 |
| HERC6, IL1R2, OLAH, RETN | | | | | | | | | |
| OAS1, IFIT1, IFI44L, ISG15, | 0.858 | 0.847 | 0.868 | 0.903 | 0.842 | 0.965 | 0.935 | 0.865 | 1 |
| HERC6, OLAH, FAM20A, MMP8 | | | | | | | | | |
| OAS1, IFI44L, SIGLEC1, ISG15, | 0.858 | 0.847 | 0.868 | 0.905 | 0.843 | 0.967 | 0.950 | 0.880 | 1 |
| SLC1A2, FAM20A, RETN, MMP8 | | | | | | | | | |
| RSAD2, OAS1, IFIT1, IFI44L, | 0.858 | 0.847 | 0.868 | 0.909 | 0.848 | 0.970 | 0.942 | 0.867 | 1 |
| ISG15, IL1R2, FAM20A, MMP8 | | | | | | | | | |
| RSAD2, OAS1, SIGLEC1, ISG15, | 0.858 | 0.848 | 0.867 | 0.911 | 0.859 | 0.964 | 0.967 | 0.931 | 1 |
| HERC6, SLC1A2, IL1R2, MMP8 | | | | | | | | | |
| RSAD2, OAS1, IFI44L, SIGLEC1, | 0.858 | 0.847 | 0.868 | 0.912 | 0.858 | 0.966 | 0.962 | 0.922 | 1 |
| HERC6, SLC1A2, IL1R2, OLAH | | | | | | | | | |
| RSAD2, OAS1, SLC1A2, IL1R2, | 0.858 | 0.848 | 0.867 | 0.915 | 0.861 | 0.968 | 0.934 | 0.852 | 1 |
| OLAH, FAM20A, RETN, MMP8 | | | | | | | | | |
| RSAD2, OAS1, IFIT1, ISG15, | 0.858 | 0.847 | 0.868 | 0.904 | 0.842 | 0.966 | 0.943 | 0.877 | 1 |
| HERC6, OLAH, FAM20A, RETN | | | | | | | | | |
| RSAD2, IFIT1, IFI44L, SIGLEC1, | 0.857 | 0.848 | 0.867 | 0.904 | 0.844 | 0.965 | 0.954 | 0.910 | 0.998 |
| ISG15, HERC6, IL1R2, OLAH | | | | | | | | | |
| RSAD2, IFIT1, IFI44L, SIGLEC1, | 0.857 | 0.848 | 0.867 | 0.912 | 0.857 | 0.968 | 0.952 | 0.907 | 0.998 |
| ISG15, SLC1A2, OLAH, MMP8 | | | | | | | | | |
| RSAD2, IFIT1, SLC1A2, IL1R2, | 0.857 | 0.848 | 0.867 | 0.912 | 0.857 | 0.966 | 0.939 | 0.864 | 1 |
| OLAH, FAM20A, RETN, MMP8 | | | | | | | | | |
| OAS1, IFIT1, ISG15, HERC6, | 0.857 | 0.847 | 0.868 | 0.909 | 0.853 | 0.965 | 0.932 | 0.853 | 1 |
| SLC1A2, OLAH, FAM20A, MMP8 | | | | | | | | | |
| RSAD2, IFI27, IFIT1, ISG15, | 0.857 | 0.847 | 0.868 | 0.914 | 0.858 | 0.969 | 0.927 | 0.843 | 1 |
| HERC6, SLC1A2, FAM20A, MMP8 | | | | | | | | | |
| RSAD2, OAS1, IFIT1, HERC6, | 0.857 | 0.847 | 0.868 | 0.903 | 0.840 | 0.966 | 0.946 | 0.879 | 1 |
| OLAH, FAM20A, RETN, MMP8 | | | | | | | | | |
| RSAD2, OAS1, HERC6, SLC1A2, | 0.857 | 0.848 | 0.867 | 0.914 | 0.861 | 0.968 | 0.929 | 0.840 | 1 |
| IL1R2, FAM20A, RETN, MMP8 | | | | | | | | | |
| RSAD2, IFI27, OAS1, ISG15, | 0.857 | 0.847 | 0.868 | 0.911 | 0.849 | 0.972 | 0.941 | 0.861 | 1 |
| SLC1A2, FAM20A, RETN, MMP8 | | | | | | | | | |
| IFI44L, SIGLEC1, ISG15, HERC6, | 0.857 | 0.848 | 0.866 | 0.910 | 0.854 | 0.965 | 0.966 | 0.931 | 1 |
| SLC1A2, IL1R2, RETN, MMP8 | | | | | | | | | |
| RSAD2, SIGLEC1, ISG15, HERC6, | 0.857 | 0.848 | 0.867 | 0.911 | 0.855 | 0.966 | 0.963 | 0.925 | 1 |
| SLC1A2, IL1R2, RETN, MMP8 | | | | | | | | | |
| IFI27, IFIT1, IFI44L, SIGLEC1, | 0.857 | 0.849 | 0.866 | 0.908 | 0.854 | 0.963 | 0.918 | 0.848 | 0.989 |
| ISG15, HERC6, SLC1A2, MMP8 | | | | | | | | | |
| RSAD2, OAS1, IFI44L, SIGLEC1, | 0.857 | 0.848 | 0.867 | 0.912 | 0.858 | 0.966 | 0.968 | 0.934 | 1 |
| SLC1A2, IL1R2, OLAH, MMP8 | | | | | | | | | |
| OAS1, IFIT1, SIGLEC1, ISG15, | 0.857 | 0.848 | 0.867 | 0.911 | 0.859 | 0.963 | 0.964 | 0.926 | 1 |
| HERC6, SLC1A2, IL1R2, MMP8 | | | | | | | | | |
| RSAD2, OAS1, IFI44L, ISG15, | 0.857 | 0.847 | 0.868 | 0.905 | 0.844 | 0.967 | 0.942 | 0.878 | 1 |
| HERC6, OLAH, FAM20A, RETN | | | | | | | | | |
| RSAD2, OAS1, IFIT1, IFI44L, | 0.857 | 0.848 | 0.867 | 0.911 | 0.856 | 0.967 | 0.968 | 0.933 | 1 |
| SIGLEC1, SLC1A2, IL1R2, OLAH | | | | | | | | | |
| OAS1, IFI44L, ISG15, SLC1A2, | 0.857 | 0.847 | 0.868 | 0.915 | 0.858 | 0.971 | 0.928 | 0.855 | 1 |
| OLAH, FAM20A, RETN, MMP8 | | | | | | | | | |
| RSAD2, OAS1, IFI44L, SIGLEC1, | 0.857 | 0.848 | 0.867 | 0.901 | 0.841 | 0.961 | 0.963 | 0.924 | 1 |
| ISG15, HERC6, IL1R2, MMP8 | | | | | | | | | |
| OAS1, IFIT1, IFI44L, SIGLEC1, | 0.857 | 0.848 | 0.867 | 0.909 | 0.852 | 0.966 | 0.930 | 0.871 | 0.990 |
| ISG15, HERC6, SLC1A2, RETN | | | | | | | | | |
| RSAD2, IFI44L, ISG15, SLC1A2, | 0.857 | 0.847 | 0.868 | 0.912 | 0.856 | 0.968 | 0.927 | 0.848 | 1 |
| IL1R2, OLAH, FAM20A, RETN | | | | | | | | | |
| OAS1, IFI44L, SLC1A2, IL1R2, | 0.857 | 0.847 | 0.868 | 0.917 | 0.864 | 0.969 | 0.932 | 0.853 | 1 |
| OLAH, FAM20A, RETN, MMP8 | | | | | | | | | |
| RSAD2, IFI44L, ISG15, HERC6, | 0.857 | 0.847 | 0.868 | 0.912 | 0.852 | 0.973 | 0.941 | 0.876 | 1 |
| OLAH, FAM20A, RETN, MMP8 | | | | | | | | | |
| RSAD2, IFIT1, ISG15, HERC6, | 0.857 | 0.847 | 0.868 | 0.911 | 0.855 | 0.966 | 0.930 | 0.845 | 1 |
| SLC1A2, IL1R2, FAM20A, RETN | | | | | | | | | |
| RSAD2, IFIT1, HERC6, SLC1A2, | 0.857 | 0.847 | 0.867 | 0.909 | 0.851 | 0.967 | 0.932 | 0.852 | 1 |
| OLAH, FAM20A, RETN, MMP8 | | | | | | | | | |
| OAS1, IFIT1, IFI44L, SLC1A2, | 0.857 | 0.847 | 0.868 | 0.913 | 0.856 | 0.970 | 0.926 | 0.851 | 1 |
| OLAH, FAM20A, RETN, MMP8 | | | | | | | | | |
| OAS1, IFI44L, SIGLEC1, ISG15, | 0.857 | 0.847 | 0.868 | 0.911 | 0.855 | 0.967 | 0.953 | 0.908 | 0.998 |
| HERC6, SLC1A2, OLAH, MMP8 | | | | | | | | | |
| RSAD2, IFIT1, IFI44L, SIGLEC1, | 0.857 | 0.848 | 0.867 | 0.914 | 0.857 | 0.970 | 0.955 | 0.911 | 1 |
| SLC1A2, OLAH, RETN, MMP8 | | | | | | | | | |
| RSAD2, IFIT1, IFI44L, ISG15, | 0.857 | 0.848 | 0.867 | 0.914 | 0.858 | 0.969 | 0.928 | 0.850 | 1 |
| SLC1A2, IL1R2, OLAH, FAM20A | | | | | | | | | |
| OAS1, IFI44L, SIGLEC1, ISG15, | 0.857 | 0.847 | 0.867 | 0.911 | 0.857 | 0.966 | 0.959 | 0.917 | 1 |
| HERC6, SLC1A2, IL1R2, OLAH | | | | | | | | | |
| RSAD2, IFIT1, IFI44L, HERC6, | 0.857 | 0.847 | 0.867 | 0.907 | 0.847 | 0.967 | 0.929 | 0.852 | 1 |
| IL1R2, OLAH, FAM20A, RETN | | | | | | | | | |
| OAS1, IFIT1, IFI44L, SIGLEC1, | 0.857 | 0.848 | 0.867 | 0.915 | 0.860 | 0.970 | 0.955 | 0.912 | 0.999 |
| SLC1A2, IL1R2, OLAH, RETN | | | | | | | | | |
| RSAD2, IFIT1, ISG15, HERC6, | 0.857 | 0.847 | 0.867 | 0.904 | 0.842 | 0.967 | 0.947 | 0.882 | 1 |
| OLAH, FAM20A, RETN, MMP8 | | | | | | | | | |
| RSAD2, IFIT1, SIGLEC1, ISG15, | 0.857 | 0.848 | 0.867 | 0.907 | 0.850 | 0.964 | 0.960 | 0.919 | 1 |
| HERC6, IL1R2, OLAH, MMP8 | | | | | | | | | |
| OAS1, IFIT1, IFI44L, SIGLEC1, | 0.857 | 0.848 | 0.867 | 0.912 | 0.857 | 0.968 | 0.961 | 0.922 | 1 |
| ISG15, SLC1A2, IL1R2, OLAH | | | | | | | | | |
| RSAD2, OAS1, IFI44L, SIGLEC1, | 0.857 | 0.847 | 0.867 | 0.911 | 0.856 | 0.967 | 0.954 | 0.910 | 0.999 |
| HERC6, SLC1A2, OLAH, MMP8 | | | | | | | | | |
| RSAD2, OAS1, IFIT1, SIGLEC1, | 0.857 | 0.847 | 0.867 | 0.903 | 0.839 | 0.966 | 0.933 | 0.875 | 0.990 |
| ISG15, HERC6, RETN, MMP8 | | | | | | | | | |
| IFIT1, IFI44L, SIGLEC1, ISG15, | 0.857 | 0.848 | 0.866 | 0.915 | 0.861 | 0.970 | 0.959 | 0.917 | 1 |
| SLC1A2, IL1R2, OLAH, RETN | | | | | | | | | |
| RSAD2, IFIT1, SIGLEC1, ISG15, | 0.857 | 0.848 | 0.867 | 0.912 | 0.856 | 0.968 | 0.960 | 0.918 | 1 |
| SLC1A2, IL1R2, OLAH, RETN | | | | | | | | | |
| RSAD2, OAS1, IFI44L, SIGLEC1, | 0.857 | 0.847 | 0.867 | 0.912 | 0.856 | 0.967 | 0.951 | 0.904 | 0.998 |
| ISG15, HERC6, SLC1A2, OLAH | | | | | | | | | |
| RSAD2, OAS1, IFIT1, IFI44L, | 0.857 | 0.847 | 0.867 | 0.912 | 0.856 | 0.968 | 0.949 | 0.899 | 0.999 |
| SIGLEC1, ISG15, SLC1A2, OLAH | | | | | | | | | |
| OAS1, IFIT1, IFI44L, ISG15, | 0.857 | 0.847 | 0.867 | 0.914 | 0.859 | 0.969 | 0.927 | 0.847 | 1 |
| SLC1A2, IL1R2, OLAH, FAM20A | | | | | | | | | |
| OAS1, IFI44L, ISG15, HERC6, | 0.857 | 0.847 | 0.867 | 0.906 | 0.843 | 0.969 | 0.950 | 0.891 | 1 |
| OLAH, FAM20A, RETN, MMP8 | | | | | | | | | |
| RSAD2, OAS1, IFIT1, SIGLEC1, | 0.857 | 0.847 | 0.867 | 0.909 | 0.852 | 0.967 | 0.933 | 0.873 | 0.992 |
| ISG15, HERC6, SLC1A2, RETN | | | | | | | | | |
| OAS1, IFIT1, HERC6, SLC1A2, | 0.857 | 0.846 | 0.868 | 0.912 | 0.858 | 0.967 | 0.927 | 0.843 | 1 |
| IL1R2, OLAH, FAM20A, RETN | | | | | | | | | |
| RSAD2, IFI44L, SIGLEC1, ISG15, | 0.857 | 0.847 | 0.866 | 0.902 | 0.841 | 0.962 | 0.962 | 0.922 | 1 |
| HERC6, IL1R2, OLAH, MMP8 | | | | | | | | | |
| OAS1, IFI44L, SIGLEC1, HERC6, | 0.857 | 0.846 | 0.868 | 0.915 | 0.862 | 0.969 | 0.957 | 0.913 | 1.000 |
| SLC1A2, IL1R2, OLAH, RETN | | | | | | | | | |
| RSAD2, IFI27, IFIT1, IFI44L, | 0.857 | 0.848 | 0.866 | 0.909 | 0.855 | 0.963 | 0.920 | 0.850 | 0.989 |
| SIGLEC1, HERC6, SLC1A2, MMP8 | | | | | | | | | |
| RSAD2, IFI27, IFIT1, SIGLEC1, | 0.857 | 0.848 | 0.866 | 0.909 | 0.854 | 0.964 | 0.917 | 0.848 | 0.987 |
| ISG15, HERC6, SLC1A2, MMP8 | | | | | | | | | |
| RSAD2, OAS1, IFIT1, IFI44L, | 0.857 | 0.847 | 0.867 | 0.901 | 0.836 | 0.966 | 0.948 | 0.882 | 1 |
| ISG15, IL1R2, OLAH, FAM20A | | | | | | | | | |
| RSAD2, IFI44L, SIGLEC1, | 0.857 | 0.847 | 0.866 | 0.914 | 0.859 | 0.968 | 0.971 | 0.939 | 1 |
| SLC1A2, IL1R2, OLAH, RETN, MMP8 | | | | | | | | | |
| RSAD2, IFI44L, SIGLEC1, ISG15, | 0.857 | 0.847 | 0.867 | 0.911 | 0.856 | 0.966 | 0.955 | 0.912 | 0.998 |
| HERC6, SLC1A2, IL1R2, OLAH | | | | | | | | | |
| RSAD2, OAS1, SIGLEC1, ISG15, | 0.857 | 0.847 | 0.867 | 0.912 | 0.856 | 0.968 | 0.968 | 0.931 | 1 |
| SLC1A2, IL1R2, OLAH, RETN | | | | | | | | | |
| OAS1, IFI44L, ISG15, SLC1A2, | 0.857 | 0.846 | 0.868 | 0.911 | 0.856 | 0.967 | 0.928 | 0.848 | 1 |
| IL1R2, OLAH, FAM20A, RETN | | | | | | | | | |
| OAS1, IFIT1, IFI44L, SIGLEC1, | 0.857 | 0.847 | 0.866 | 0.910 | 0.857 | 0.963 | 0.959 | 0.918 | 1.000 |
| ISG15, HERC6, SLC1A2, IL1R2 | | | | | | | | | |
| OAS1, IFI44L, SIGLEC1, ISG15, | 0.857 | 0.847 | 0.867 | 0.911 | 0.853 | 0.969 | 0.959 | 0.915 | 1 |
| SLC1A2, OLAH, RETN, MMP8 | | | | | | | | | |
| RSAD2, IFI44L, ISG15, SLC1A2, | 0.857 | 0.847 | 0.867 | 0.913 | 0.856 | 0.970 | 0.926 | 0.852 | 1.000 |
| OLAH, FAM20A, RETN, MMP8 | | | | | | | | | |
| RSAD2, OAS1, IFI44L, IL1R2, | 0.857 | 0.846 | 0.867 | 0.909 | 0.848 | 0.970 | 0.945 | 0.876 | 1 |
| OLAH, FAM20A, RETN, MMP8 | | | | | | | | | |
| RSAD2, IFI44L, HERC6, SLC1A2, | 0.857 | 0.847 | 0.867 | 0.918 | 0.865 | 0.970 | 0.928 | 0.838 | 1 |
| IL1R2, FAM20A, RETN, MMP8 | | | | | | | | | |
| RSAD2, IFI44L, ISG15, HERC6, | 0.857 | 0.846 | 0.868 | 0.913 | 0.860 | 0.967 | 0.927 | 0.840 | 1 |
| SLC1A2, IL1R2, FAM20A, RETN | | | | | | | | | |
| RSAD2, OAS1, IFI44L, ISG15, | 0.857 | 0.846 | 0.868 | 0.905 | 0.843 | 0.966 | 0.942 | 0.879 | 1 |
| HERC6, OLAH, FAM20A, MMP8 | | | | | | | | | |
| RSAD2, IFI27, OAS1, IFIT1, | 0.857 | 0.846 | 0.867 | 0.913 | 0.858 | 0.968 | 0.924 | 0.839 | 1 |
| HERC6, SLC1A2, FAM20A, MMP8 | | | | | | | | | |
| RSAD2, OAS1, IFIT1, SIGLEC1, | 0.857 | 0.847 | 0.867 | 0.913 | 0.857 | 0.970 | 0.961 | 0.918 | 1 |
| SLC1A2, OLAH, RETN, MMP8 | | | | | | | | | |
| RSAD2, OAS1, IFI44L, SIGLEC1, | 0.857 | 0.847 | 0.867 | 0.900 | 0.836 | 0.965 | 0.958 | 0.914 | 1 |
| HERC6, IL1R2, OLAH, RETN | | | | | | | | | |
| RSAD2, IFIT1, ISG15, HERC6, | 0.857 | 0.846 | 0.867 | 0.909 | 0.853 | 0.965 | 0.929 | 0.849 | 1 |
| SLC1A2, OLAH, FAM20A, MMP8 | | | | | | | | | |
| RSAD2, OAS1, IFIT1, SIGLEC1, | 0.857 | 0.847 | 0.866 | 0.908 | 0.855 | 0.962 | 0.966 | 0.929 | 1 |
| HERC6, SLC1A2, IL1R2, MMP8 | | | | | | | | | |
| OAS1, IFIT1, IFI44L, IL1R2, | 0.857 | 0.847 | 0.867 | 0.906 | 0.843 | 0.969 | 0.947 | 0.878 | 1 |
| OLAH, FAM20A, RETN, MMP8 | | | | | | | | | |
| RSAD2, OAS1, SIGLEC1, ISG15, | 0.857 | 0.846 | 0.867 | 0.900 | 0.834 | 0.965 | 0.964 | 0.927 | 1 |
| HERC6, OLAH, RETN, MMP8 | | | | | | | | | |
| OAS1, IFI44L, SIGLEC1, SLC1A2, | 0.857 | 0.847 | 0.867 | 0.913 | 0.859 | 0.967 | 0.967 | 0.932 | 1 |
| IL1R2, OLAH, RETN, MMP8 | | | | | | | | | |
| RSAD2, IFI44L, SIGLEC1, HERC6, | 0.857 | 0.846 | 0.867 | 0.914 | 0.860 | 0.968 | 0.957 | 0.914 | 0.999 |
| SLC1A2, IL1R2, OLAH, RETN | | | | | | | | | |
| RSAD2, OAS1, IFI44L, SIGLEC1, | 0.857 | 0.847 | 0.867 | 0.914 | 0.857 | 0.970 | 0.952 | 0.903 | 1 |
| HERC6, SLC1A2, OLAH, RETN | | | | | | | | | |
| IFIT1, IFI44L, ISG15, SLC1A2, | 0.857 | 0.847 | 0.866 | 0.912 | 0.858 | 0.966 | 0.932 | 0.848 | 1 |
| IL1R2, FAM20A, RETN, MMP8 | | | | | | | | | |
| IFIT1, IFI44L, SIGLEC1, ISG15, | 0.857 | 0.847 | 0.866 | 0.908 | 0.855 | 0.962 | 0.964 | 0.927 | 1 |
| HERC6, SLC1A2, IL1R2, MMP8 | | | | | | | | | |
| RSAD2, OAS1, IFI44L, SIGLEC1, | 0.856 | 0.847 | 0.866 | 0.911 | 0.856 | 0.966 | 0.957 | 0.909 | 1 |
| HERC6, SLC1A2, IL1R2, RETN | | | | | | | | | |
| RSAD2, OAS1, IFI44L, SLC1A2, | 0.856 | 0.846 | 0.867 | 0.914 | 0.857 | 0.971 | 0.926 | 0.851 | 1 |
| OLAH, FAM20A, RETN, MMP8 | | | | | | | | | |
| RSAD2, IFI44L, SIGLEC1, ISG15, | 0.856 | 0.847 | 0.866 | 0.911 | 0.856 | 0.967 | 0.947 | 0.898 | 0.995 |
| HERC6, SLC1A2, OLAH, MMP8 | | | | | | | | | |
| RSAD2, IFIT1, SIGLEC1, ISG15, | 0.856 | 0.847 | 0.866 | 0.911 | 0.855 | 0.968 | 0.958 | 0.915 | 1 |
| SLC1A2, OLAH, RETN, MMP8 | | | | | | | | | |
| RSAD2, OAS1, IFI44L, SIGLEC1, | 0.856 | 0.846 | 0.867 | 0.903 | 0.841 | 0.966 | 0.943 | 0.870 | 1 |
| ISG15, SLC1A2, FAM20A, MMP8 | | | | | | | | | |
| RSAD2, OAS1, IFIT1, IFI44L, | 0.856 | 0.847 | 0.866 | 0.912 | 0.855 | 0.968 | 0.949 | 0.896 | 1 |
| SIGLEC1, SLC1A2, OLAH, RETN | | | | | | | | | |
| OAS1, IFIT1, SIGLEC1, ISG15, | 0.856 | 0.846 | 0.866 | 0.910 | 0.854 | 0.967 | 0.935 | 0.878 | 0.992 |
| HERC6, SLC1A2, RETN, MMP8 | | | | | | | | | |
| RSAD2, OAS1, IFIT1, ISG15, | 0.856 | 0.846 | 0.866 | 0.909 | 0.851 | 0.967 | 0.933 | 0.853 | 1 |
| SLC1A2, IL1R2, OLAH, FAM20A | | | | | | | | | |
| RSAD2, IFIT1, IFI44L, SLC1A2, | 0.856 | 0.846 | 0.866 | 0.911 | 0.856 | 0.967 | 0.926 | 0.845 | 1 |
| IL1R2, OLAH, FAM20A, RETN | | | | | | | | | |
| RSAD2, IFIT1, IFI44L, SIGLEC1, | 0.856 | 0.847 | 0.865 | 0.907 | 0.851 | 0.964 | 0.952 | 0.907 | 0.997 |
| HERC6, SLC1A2, IL1R2, RETN | | | | | | | | | |
| RSAD2, IFIT1, IFI44L, SIGLEC1, | 0.856 | 0.847 | 0.866 | 0.903 | 0.840 | 0.966 | 0.943 | 0.894 | 0.993 |
| ISG15, HERC6, OLAH, RETN | | | | | | | | | |
| RSAD2, OAS1, IFIT1, ISG15, | 0.856 | 0.846 | 0.867 | 0.910 | 0.854 | 0.966 | 0.930 | 0.851 | 1 |
| HERC6, SLC1A2, OLAH, FAM20A | | | | | | | | | |
| RSAD2, IFIT1, HERC6, SLC1A2, | 0.856 | 0.846 | 0.866 | 0.912 | 0.857 | 0.967 | 0.928 | 0.844 | 1 |
| IL1R2, OLAH, FAM20A, RETN | | | | | | | | | |
| RSAD2, IFI44L, SIGLEC1, ISG15, | 0.856 | 0.847 | 0.865 | 0.911 | 0.856 | 0.965 | 0.970 | 0.937 | 1 |
| SLC1A2, IL1R2, OLAH, MMP8 | | | | | | | | | |
| RSAD2, IFIT1, SIGLEC1, ISG15, | 0.856 | 0.847 | 0.865 | 0.908 | 0.854 | 0.963 | 0.965 | 0.929 | 1 |
| HERC6, SLC1A2, IL1R2, MMP8 | | | | | | | | | |
| OAS1, ISG15, SLC1A2, IL1R2, | 0.856 | 0.846 | 0.866 | 0.915 | 0.861 | 0.968 | 0.930 | 0.848 | 1 |
| OLAH, FAM20A, RETN, MMP8 | | | | | | | | | |
| OAS1, IFI44L, SIGLEC1, ISG15, | 0.856 | 0.847 | 0.866 | 0.914 | 0.860 | 0.967 | 0.966 | 0.931 | 1 |
| SLC1A2, IL1R2, OLAH, MMP8 | | | | | | | | | |
| RSAD2, OAS1, IFI44L, SLC1A2, | 0.856 | 0.845 | 0.867 | 0.913 | 0.858 | 0.968 | 0.928 | 0.848 | 1 |
| IL1R2, OLAH, FAM20A, RETN | | | | | | | | | |
| RSAD2, OAS1, SIGLEC1, ISG15, | 0.856 | 0.846 | 0.866 | 0.910 | 0.853 | 0.968 | 0.961 | 0.919 | 1 |
| SLC1A2, OLAH, RETN, MMP8 | | | | | | | | | |
| OAS1, IFIT1, IFI44L, SLC1A2, | 0.856 | 0.846 | 0.866 | 0.913 | 0.858 | 0.968 | 0.928 | 0.849 | 1 |
| IL1R2, OLAH, FAM20A, RETN | | | | | | | | | |
| RSAD2, OAS1, ISG15, SLC1A2, | 0.856 | 0.846 | 0.866 | 0.910 | 0.854 | 0.967 | 0.928 | 0.844 | 1 |
| IL1R2, OLAH, FAM20A, RETN | | | | | | | | | |
| RSAD2, IFIT1, IFI44L, SIGLEC1, | 0.856 | 0.847 | 0.865 | 0.910 | 0.854 | 0.966 | 0.964 | 0.927 | 1 |
| ISG15, SLC1A2, IL1R2, OLAH | | | | | | | | | |
| RSAD2, OAS1, IFIT1, ISG15, | 0.856 | 0.846 | 0.866 | 0.908 | 0.846 | 0.970 | 0.947 | 0.875 | 1 |
| IL1R2, FAM20A, RETN, MMP8 | | | | | | | | | |
| RSAD2, OAS1, IFIT1, IFI44L, | 0.856 | 0.847 | 0.865 | 0.901 | 0.841 | 0.962 | 0.960 | 0.919 | 1 |
| SIGLEC1, IL1R2, OLAH, MMP8 | | | | | | | | | |
| RSAD2, IFI44L, SIGLEC1, HERC6, | 0.856 | 0.847 | 0.865 | 0.910 | 0.854 | 0.965 | 0.964 | 0.927 | 1 |
| SLC1A2, IL1R2, RETN, MMP8 | | | | | | | | | |
| RSAD2, IFI44L, SIGLEC1, HERC6, | 0.856 | 0.846 | 0.866 | 0.911 | 0.853 | 0.969 | 0.953 | 0.909 | 0.997 |
| SLC1A2, OLAH, RETN, MMP8 | | | | | | | | | |
| RSAD2, ISG15, SLC1A2, IL1R2, | 0.856 | 0.846 | 0.866 | 0.914 | 0.861 | 0.967 | 0.932 | 0.849 | 1 |
| OLAH, FAM20A, RETN, MMP8 | | | | | | | | | |
| RSAD2, IFIT1, IFI44L, SIGLEC1, | 0.856 | 0.847 | 0.865 | 0.908 | 0.853 | 0.962 | 0.963 | 0.925 | 1 |
| HERC6, SLC1A2, IL1R2, MMP8 | | | | | | | | | |
| RSAD2, IFIT1, IFI44L, SIGLEC1, | 0.856 | 0.847 | 0.865 | 0.913 | 0.857 | 0.969 | 0.962 | 0.923 | 1 |
| SLC1A2, IL1R2, OLAH, RETN | | | | | | | | | |
| RSAD2, OAS1, IFI44L, SIGLEC1, | 0.856 | 0.846 | 0.866 | 0.912 | 0.856 | 0.967 | 0.966 | 0.928 | 1 |
| ISG15, SLC1A2, IL1R2, OLAH | | | | | | | | | |
| RSAD2, OAS1, IFIT1, SIGLEC1, | 0.856 | 0.846 | 0.866 | 0.901 | 0.839 | 0.964 | 0.965 | 0.929 | 1 |
| IL1R2, OLAH, RETN, MMP8 | | | | | | | | | |
| OAS1, IFI44L, SIGLEC1, ISG15, | 0.856 | 0.846 | 0.866 | 0.914 | 0.858 | 0.969 | 0.962 | 0.922 | 1 |
| SLC1A2, IL1R2, OLAH, RETN | | | | | | | | | |
| OAS1, IFIT1, ISG15, SLC1A2, | 0.856 | 0.845 | 0.866 | 0.912 | 0.855 | 0.968 | 0.935 | 0.856 | 1 |
| IL1R2, OLAH, FAM20A, RETN | | | | | | | | | |
| OAS1, IFIT1, IFI44L, HERC6, | 0.856 | 0.846 | 0.866 | 0.916 | 0.864 | 0.967 | 0.925 | 0.836 | 1 |
| SLC1A2, IL1R2, FAM20A, RETN | | | | | | | | | |
| OAS1, IFIT1, IFI44L, SIGLEC1, | 0.856 | 0.847 | 0.865 | 0.908 | 0.849 | 0.967 | 0.927 | 0.868 | 0.987 |
| HERC6, SLC1A2, RETN, MMP8 | | | | | | | | | |
| RSAD2, OAS1, IFIT1, IFI44L, | 0.856 | 0.846 | 0.865 | 0.908 | 0.852 | 0.963 | 0.964 | 0.925 | 1 |
| SIGLEC1, HERC6, SLC1A2, IL1R2 | | | | | | | | | |
| RSAD2, OAS1, IFI44L, SIGLEC1, | 0.856 | 0.846 | 0.866 | 0.908 | 0.853 | 0.963 | 0.962 | 0.921 | 1 |
| ISG15, HERC6, SLC1A2, IL1R2 | | | | | | | | | |
| RSAD2, OAS1, IFI44L, HERC6, | 0.856 | 0.845 | 0.866 | 0.915 | 0.863 | 0.967 | 0.926 | 0.838 | 1 |
| SLC1A2, IL1R2, FAM20A, RETN | | | | | | | | | |
| OAS1, IFIT1, SIGLEC1, ISG15, | 0.856 | 0.846 | 0.866 | 0.902 | 0.840 | 0.964 | 0.964 | 0.928 | 1 |
| IL1R2, OLAH, RETN, MMP8 | | | | | | | | | |
| OAS1, IFIT1, IFI44L, ISG15, | 0.856 | 0.845 | 0.866 | 0.911 | 0.853 | 0.969 | 0.922 | 0.844 | 1.000 |
| SLC1A2, OLAH, FAM20A, RETN | | | | | | | | | |
| RSAD2, OAS1, IFIT1, ISG15, | 0.856 | 0.846 | 0.865 | 0.915 | 0.862 | 0.968 | 0.936 | 0.855 | 1 |
| SLC1A2, IL1R2, FAM20A, MMP8 | | | | | | | | | |
| RSAD2, OAS1, IFIT1, SIGLEC1, | 0.856 | 0.846 | 0.865 | 0.911 | 0.855 | 0.968 | 0.958 | 0.910 | 1 |
| ISG15, SLC1A2, OLAH, RETN | | | | | | | | | |
| RSAD2, OAS1, IFIT1, IFI44L, | 0.856 | 0.846 | 0.866 | 0.911 | 0.853 | 0.969 | 0.921 | 0.842 | 0.999 |
| ISG15, SLC1A2, OLAH, FAM20A | | | | | | | | | |
| RSAD2, IFIT1, ISG15, HERC6, | 0.856 | 0.845 | 0.866 | 0.911 | 0.854 | 0.968 | 0.930 | 0.849 | 1 |
| SLC1A2, OLAH, FAM20A, RETN | | | | | | | | | |
| RSAD2, IFI44L, SIGLEC1, ISG15, | 0.856 | 0.846 | 0.865 | 0.914 | 0.857 | 0.970 | 0.947 | 0.898 | 0.995 |
| HERC6, SLC1A2, OLAH, RETN | | | | | | | | | |
| RSAD2, IFIT1, IFI44L, ISG15, | 0.856 | 0.846 | 0.866 | 0.908 | 0.848 | 0.969 | 0.925 | 0.845 | 1 |
| HERC6, FAM20A, RETN, MMP8 | | | | | | | | | |
| OAS1, IFIT1, IFI44L, SIGLEC1, | 0.856 | 0.846 | 0.865 | 0.912 | 0.858 | 0.966 | 0.970 | 0.936 | 1 |
| SLC1A2, IL1R2, RETN, MMP8 | | | | | | | | | |
| RSAD2, OAS1, IFIT1, IFI44L, | 0.856 | 0.846 | 0.865 | 0.910 | 0.853 | 0.968 | 0.928 | 0.868 | 0.989 |
| SIGLEC1, HERC6, SLC1A2, RETN | | | | | | | | | |
| RSAD2, OAS1, IFIT1, SLC1A2, | 0.856 | 0.846 | 0.866 | 0.911 | 0.854 | 0.967 | 0.935 | 0.855 | 1 |
| IL1R2, OLAH, FAM20A, RETN | | | | | | | | | |
| OAS1, IFI44L, SIGLEC1, ISG15, | 0.856 | 0.846 | 0.865 | 0.910 | 0.858 | 0.963 | 0.963 | 0.924 | 1 |
| HERC6, SLC1A2, IL1R2, MMP8 | | | | | | | | | |
| RSAD2, OAS1, IFIT1, IFI44L, | 0.855 | 0.846 | 0.865 | 0.897 | 0.832 | 0.963 | 0.958 | 0.916 | 0.999 |
| SIGLEC1, ISG15, IL1R2, OLAH | | | | | | | | | |
| OAS1, IFIT1, SIGLEC1, ISG15, | 0.855 | 0.846 | 0.865 | 0.912 | 0.858 | 0.966 | 0.971 | 0.938 | 1 |
| SLC1A2, IL1R2, RETN, MMP8 | | | | | | | | | |
| RSAD2, IFI27, OAS1, IFIT1, | 0.855 | 0.846 | 0.865 | 0.905 | 0.846 | 0.964 | 0.932 | 0.861 | 1 |
| SIGLEC1, ISG15, SLC1A2, MMP8 | | | | | | | | | |
| RSAD2, OAS1, IFI44L, SIGLEC1, | 0.855 | 0.846 | 0.865 | 0.911 | 0.855 | 0.967 | 0.968 | 0.931 | 1 |
| SLC1A2, IL1R2, OLAH, RETN | | | | | | | | | |
| RSAD2, IFIT1, IFI44L, ISG15, | 0.855 | 0.846 | 0.865 | 0.911 | 0.859 | 0.964 | 0.928 | 0.842 | 1 |
| SLC1A2, IL1R2, FAM20A, MMP8 | | | | | | | | | |
| RSAD2, OAS1, IFI44L, SIGLEC1, | 0.855 | 0.845 | 0.865 | 0.911 | 0.856 | 0.967 | 0.952 | 0.906 | 0.998 |
| ISG15, SLC1A2, OLAH, MMP8 | | | | | | | | | |
| RSAD2, IFIT1, IFI44L, SIGLEC1, | 0.855 | 0.846 | 0.865 | 0.914 | 0.858 | 0.970 | 0.946 | 0.894 | 0.997 |
| ISG15, SLC1A2, OLAH, RETN | | | | | | | | | |
| OAS1, IFIT1, IFI44L, ISG15, | 0.855 | 0.845 | 0.865 | 0.914 | 0.861 | 0.966 | 0.929 | 0.840 | 1 |
| SLC1A2, IL1R2, FAM20A, MMP8 | | | | | | | | | |
| RSAD2, OAS1, IFIT1, SLC1A2, | 0.855 | 0.846 | 0.865 | 0.914 | 0.859 | 0.968 | 0.936 | 0.856 | 1 |
| IL1R2, FAM20A, RETN, MMP8 | | | | | | | | | |
| RSAD2, OAS1, IFI44L, SIGLEC1, | 0.855 | 0.845 | 0.866 | 0.905 | 0.843 | 0.967 | 0.945 | 0.873 | 1 |
| ISG15, SLC1A2, FAM20A, RETN | | | | | | | | | |
| RSAD2, OAS1, IFIT1, ISG15, | 0.855 | 0.845 | 0.866 | 0.901 | 0.839 | 0.963 | 0.941 | 0.872 | 1 |
| HERC6, OLAH, FAM20A, MMP8 | | | | | | | | | |
| RSAD2, IFI44L, SIGLEC1, ISG15, | 0.855 | 0.846 | 0.865 | 0.908 | 0.852 | 0.964 | 0.958 | 0.915 | 1 |
| HERC6, SLC1A2, IL1R2, RETN | | | | | | | | | |
| RSAD2, IFIT1, IFI44L, SIGLEC1, | 0.855 | 0.846 | 0.864 | 0.906 | 0.851 | 0.962 | 0.960 | 0.920 | 1.000 |
| ISG15, HERC6, SLC1A2, IL1R2 | | | | | | | | | |
| RSAD2, OAS1, IFIT1, HERC6, | 0.855 | 0.845 | 0.865 | 0.912 | 0.857 | 0.966 | 0.930 | 0.845 | 1 |
| SLC1A2, IL1R2, FAM20A, RETN | | | | | | | | | |
| OAS1, IFIT1, IFI44L, SIGLEC1, | 0.855 | 0.846 | 0.865 | 0.903 | 0.843 | 0.963 | 0.958 | 0.916 | 1.000 |
| ISG15, IL1R2, OLAH, MMP8 | | | | | | | | | |
| OAS1, IFIT1, ISG15, SLC1A2, | 0.855 | 0.845 | 0.865 | 0.911 | 0.855 | 0.967 | 0.937 | 0.858 | 1 |
| IL1R2, FAM20A, RETN, MMP8 | | | | | | | | | |
| RSAD2, OAS1, IFI44L, ISG15, | 0.855 | 0.845 | 0.866 | 0.914 | 0.863 | 0.965 | 0.928 | 0.838 | 1 |
| SLC1A2, IL1R2, FAM20A, MMP8 | | | | | | | | | |
| OAS1, IFIT1, IFI44L, SIGLEC1, | 0.855 | 0.845 | 0.865 | 0.901 | 0.837 | 0.965 | 0.962 | 0.923 | 1 |
| ISG15, IL1R2, RETN, MMP8 | | | | | | | | | |
| RSAD2, IFI27, OAS1, IFIT1, | 0.855 | 0.846 | 0.864 | 0.904 | 0.845 | 0.963 | 0.930 | 0.859 | 1 |
| IFI44L, SIGLEC1, SLC1A2, MMP8 | | | | | | | | | |
| OAS1, IFIT1, IFI44L, ISG15, | 0.855 | 0.845 | 0.866 | 0.908 | 0.850 | 0.967 | 0.920 | 0.830 | 1 |
| HERC6, SLC1A2, FAM20A, RETN | | | | | | | | | |
| RSAD2, IFIT1, IFI44L, ISG15, | 0.855 | 0.845 | 0.865 | 0.905 | 0.844 | 0.966 | 0.952 | 0.892 | 1 |
| IL1R2, OLAH, FAM20A, MMP8 | | | | | | | | | |
| RSAD2, IFI44L, SIGLEC1, ISG15, | 0.855 | 0.845 | 0.865 | 0.903 | 0.840 | 0.966 | 0.959 | 0.917 | 1 |
| HERC6, IL1R2, OLAH, RETN | | | | | | | | | |
| OAS1, IFIT1, IFI44L, SIGLEC1, | 0.855 | 0.845 | 0.865 | 0.900 | 0.839 | 0.962 | 0.943 | 0.893 | 0.994 |
| ISG15, HERC6, OLAH, MMP8 | | | | | | | | | |
| IFI27, OAS1, IFIT1, IFI44L, | 0.855 | 0.846 | 0.864 | 0.904 | 0.846 | 0.963 | 0.929 | 0.861 | 0.998 |
| SIGLEC1, ISG15, SLC1A2, MMP8 | | | | | | | | | |
| IFIT1, IFI44L, SIGLEC1, ISG15, | 0.855 | 0.845 | 0.865 | 0.902 | 0.840 | 0.964 | 0.964 | 0.927 | 1 |
| IL1R2, OLAH, RETN, MMP8 | | | | | | | | | |
| IFIT1, IFI44L, ISG15, IL1R2, | 0.855 | 0.845 | 0.865 | 0.906 | 0.844 | 0.968 | 0.951 | 0.889 | 1 |
| OLAH, FAM20A, RETN, MMP8 | | | | | | | | | |
| RSAD2, IFIT1, IFI44L, SIGLEC1, | 0.855 | 0.845 | 0.865 | 0.902 | 0.839 | 0.965 | 0.968 | 0.934 | 1 |
| IL1R2, OLAH, RETN, MMP8 | | | | | | | | | |
| RSAD2, OAS1, IFI44L, ISG15, | 0.855 | 0.844 | 0.866 | 0.908 | 0.847 | 0.969 | 0.935 | 0.859 | 1 |
| HERC6, FAM20A, RETN, MMP8 | | | | | | | | | |
| RSAD2, OAS1, IFI44L, SIGLEC1, | 0.855 | 0.845 | 0.865 | 0.907 | 0.853 | 0.961 | 0.967 | 0.931 | 1 |
| HERC6, SLC1A2, IL1R2, MMP8 | | | | | | | | | |
| RSAD2, OAS1, IFIT1, ISG15, | 0.855 | 0.845 | 0.865 | 0.901 | 0.836 | 0.966 | 0.940 | 0.867 | 1 |
| IL1R2, OLAH, FAM20A, RETN | | | | | | | | | |
| RSAD2, IFI27, OAS1, IFIT1, | 0.855 | 0.846 | 0.864 | 0.901 | 0.844 | 0.958 | 0.896 | 0.816 | 0.976 |
| IFI44L, ISG15, HERC6, MMP8 | | | | | | | | | |
| RSAD2, IFI27, IFIT1, IFI44L, | 0.855 | 0.844 | 0.866 | 0.904 | 0.840 | 0.968 | 0.936 | 0.855 | 1 |
| ISG15, SLC1A2, FAM20A, MMP8 | | | | | | | | | |
| OAS1, IFIT1, IFI44L, SLC1A2, | 0.855 | 0.845 | 0.865 | 0.912 | 0.858 | 0.966 | 0.932 | 0.846 | 1 |
| IL1R2, FAM20A, RETN, MMP8 | | | | | | | | | |
| RSAD2, IFIT1, IFI44L, HERC6, | 0.855 | 0.845 | 0.865 | 0.914 | 0.862 | 0.966 | 0.923 | 0.834 | 1 |
| SLC1A2, IL1R2, FAM20A, RETN | | | | | | | | | |
| RSAD2, OAS1, IFI44L, SIGLEC1, | 0.855 | 0.845 | 0.865 | 0.911 | 0.854 | 0.969 | 0.959 | 0.915 | 1 |
| SLC1A2, OLAH, RETN, MMP8 | | | | | | | | | |
| RSAD2, IFI44L, SIGLEC1, ISG15, | 0.855 | 0.845 | 0.864 | 0.912 | 0.856 | 0.968 | 0.962 | 0.923 | 1 |
| SLC1A2, IL1R2, OLAH, RETN | | | | | | | | | |
| OAS1, IFI44L, SIGLEC1, ISG15, | 0.855 | 0.844 | 0.865 | 0.899 | 0.833 | 0.965 | 0.964 | 0.927 | 1 |
| HERC6, OLAH, RETN, MMP8 | | | | | | | | | |
| RSAD2, OAS1, IFIT1, IFI44L, | 0.855 | 0.845 | 0.865 | 0.897 | 0.831 | 0.964 | 0.961 | 0.919 | 1 |
| SIGLEC1, IL1R2, OLAH, RETN | | | | | | | | | |
| RSAD2, OAS1, IFIT1, IFI44L, | 0.855 | 0.845 | 0.865 | 0.900 | 0.836 | 0.964 | 0.970 | 0.937 | 1 |
| SIGLEC1, IL1R2, RETN, MMP8 | | | | | | | | | |
| RSAD2, OAS1, IFIT1, HERC6, | 0.855 | 0.844 | 0.865 | 0.911 | 0.854 | 0.967 | 0.934 | 0.854 | 1 |
| SLC1A2, OLAH, FAM20A, RETN | | | | | | | | | |
| RSAD2, OAS1, IFIT1, ISG15, | 0.855 | 0.845 | 0.865 | 0.908 | 0.850 | 0.967 | 0.940 | 0.869 | 1 |
| SLC1A2, OLAH, FAM20A, MMP8 | | | | | | | | | |
| RSAD2, OAS1, IFI44L, ISG15, | 0.855 | 0.844 | 0.865 | 0.911 | 0.852 | 0.970 | 0.923 | 0.847 | 0.999 |
| SLC1A2, OLAH, FAM20A, RETN | | | | | | | | | |
| IFI27, OAS1, IFIT1, IFI44L, ISG15, | 0.855 | 0.843 | 0.866 | 0.903 | 0.838 | 0.967 | 0.932 | 0.850 | 1 |
| SLC1A2, FAM20A, MMP8 | | | | | | | | | |
| RSAD2, OAS1, IFIT1, IFI44L, | 0.855 | 0.845 | 0.864 | 0.899 | 0.837 | 0.961 | 0.941 | 0.889 | 0.993 |
| SIGLEC1, HERC6, OLAH, MMP8 | | | | | | | | | |
| RSAD2, IFI27, OAS1, IFI44L, | 0.855 | 0.845 | 0.864 | 0.905 | 0.846 | 0.964 | 0.933 | 0.862 | 1 |
| SIGLEC1, ISG15, SLC1A2, MMP8 | | | | | | | | | |
| RSAD2, IFIT1, IFI44L, ISG15, | 0.855 | 0.844 | 0.865 | 0.910 | 0.852 | 0.967 | 0.922 | 0.837 | 1 |
| HERC6, SLC1A2, FAM20A, RETN | | | | | | | | | |
| RSAD2, OAS1, IFI44L, SIGLEC1, | 0.855 | 0.844 | 0.865 | 0.901 | 0.838 | 0.965 | 0.967 | 0.932 | 1 |
| IL1R2, OLAH, RETN, MMP8 | | | | | | | | | |
| RSAD2, IFI27, OAS1, IFI44L, | 0.855 | 0.843 | 0.866 | 0.904 | 0.840 | 0.968 | 0.934 | 0.853 | 1 |
| ISG15, SLC1A2, FAM20A, MMP8 | | | | | | | | | |
| RSAD2, OAS1, IFIT1, IFI44L, | 0.855 | 0.844 | 0.865 | 0.908 | 0.846 | 0.970 | 0.946 | 0.871 | 1 |
| IL1R2, FAM20A, RETN, MMP8 | | | | | | | | | |
| OAS1, IFI44L, ISG15, IL1R2, | 0.855 | 0.844 | 0.865 | 0.906 | 0.844 | 0.968 | 0.947 | 0.878 | 1 |
| OLAH, FAM20A, RETN, MMP8 | | | | | | | | | |
| OAS1, IFIT1, IFI44L, ISG15, | 0.855 | 0.845 | 0.865 | 0.907 | 0.844 | 0.969 | 0.927 | 0.848 | 1 |
| HERC6, FAM20A, RETN, MMP8 | | | | | | | | | |
| RSAD2, IFI44L, SIGLEC1, ISG15, | 0.855 | 0.846 | 0.864 | 0.907 | 0.852 | 0.962 | 0.963 | 0.925 | 1 |
| HERC6, SLC1A2, IL1R2, MMP8 | | | | | | | | | |
| RSAD2, OAS1, IFIT1, IFI44L, | 0.855 | 0.845 | 0.864 | 0.900 | 0.833 | 0.967 | 0.925 | 0.865 | 0.985 |
| SIGLEC1, HERC6, RETN, MMP8 | | | | | | | | | |
| RSAD2, IFIT1, IFI44L, SIGLEC1, | 0.855 | 0.845 | 0.864 | 0.901 | 0.840 | 0.962 | 0.959 | 0.917 | 1 |
| ISG15, IL1R2, OLAH, MMP8 | | | | | | | | | |
| RSAD2, IFIT1, ISG15, SLC1A2, | 0.854 | 0.845 | 0.864 | 0.908 | 0.852 | 0.965 | 0.936 | 0.860 | 1 |
| IL1R2, OLAH, FAM20A, RETN | | | | | | | | | |
| RSAD2, OAS1, IFI44L, SIGLEC1, | 0.854 | 0.845 | 0.864 | 0.911 | 0.854 | 0.968 | 0.954 | 0.905 | 1 |
| ISG15, SLC1A2, OLAH, RETN | | | | | | | | | |
| RSAD2, OAS1, SIGLEC1, ISG15, | 0.854 | 0.844 | 0.865 | 0.901 | 0.838 | 0.964 | 0.970 | 0.936 | 1 |
| IL1R2, OLAH, RETN, MMP8 | | | | | | | | | |
| RSAD2, IFI27, IFI44L, SIGLEC1, | 0.854 | 0.845 | 0.863 | 0.906 | 0.851 | 0.961 | 0.923 | 0.854 | 0.992 |
| ISG15, HERC6, SLC1A2, MMP8 | | | | | | | | | |
| OAS1, IFIT1, IFI44L, SIGLEC1, | 0.854 | 0.845 | 0.864 | 0.909 | 0.854 | 0.964 | 0.965 | 0.927 | 1 |
| ISG15, SLC1A2, IL1R2, RETN | | | | | | | | | |
| RSAD2, IFIT1, IFI44L, SLC1A2, | 0.854 | 0.845 | 0.864 | 0.911 | 0.857 | 0.966 | 0.932 | 0.848 | 1 |
| IL1R2, FAM20A, RETN, MMP8 | | | | | | | | | |
| RSAD2, IFIT1, ISG15, SLC1A2, | 0.854 | 0.845 | 0.864 | 0.911 | 0.855 | 0.966 | 0.937 | 0.859 | 1 |
| IL1R2, FAM20A, RETN, MMP8 | | | | | | | | | |
| RSAD2, IFIT1, IFI44L, ISG15, | 0.854 | 0.844 | 0.864 | 0.909 | 0.850 | 0.968 | 0.921 | 0.844 | 0.997 |
| SLC1A2, OLAH, FAM20A, RETN | | | | | | | | | |
| IFIT1, IFI44L, SIGLEC1, ISG15, | 0.854 | 0.845 | 0.863 | 0.912 | 0.858 | 0.966 | 0.967 | 0.932 | 1 |
| SLC1A2, IL1R2, RETN, MMP8 | | | | | | | | | |
| RSAD2, IFI27, OAS1, IFIT1, | 0.854 | 0.843 | 0.865 | 0.903 | 0.839 | 0.967 | 0.934 | 0.853 | 1 |
| IFI44L, SLC1A2, FAM20A, MMP8 | | | | | | | | | |
| RSAD2, IFI44L, SIGLEC1, ISG15, | 0.854 | 0.844 | 0.864 | 0.911 | 0.853 | 0.968 | 0.958 | 0.916 | 0.999 |
| SLC1A2, OLAH, RETN, MMP8 | | | | | | | | | |
| RSAD2, OAS1, IFI44L, ISG15, | 0.854 | 0.843 | 0.865 | 0.905 | 0.844 | 0.966 | 0.929 | 0.850 | 1 |
| HERC6, SLC1A2, FAM20A, RETN | | | | | | | | | |
| RSAD2, OAS1, SIGLEC1, ISG15, | 0.854 | 0.844 | 0.865 | 0.905 | 0.845 | 0.965 | 0.951 | 0.901 | 1 |
| HERC6, SLC1A2, RETN, MMP8 | | | | | | | | | |
| RSAD2, OAS1, IFIT1, SIGLEC1, | 0.854 | 0.845 | 0.864 | 0.911 | 0.857 | 0.965 | 0.974 | 0.944 | 1 |
| SLC1A2, IL1R2, RETN, MMP8 | | | | | | | | | |
| RSAD2, OAS1, IFI44L, SIGLEC1, | 0.854 | 0.845 | 0.863 | 0.906 | 0.849 | 0.963 | 0.967 | 0.929 | 1 |
| ISG15, SLC1A2, IL1R2, RETN | | | | | | | | | |
| RSAD2, OAS1, IFIT1, SIGLEC1, | 0.854 | 0.844 | 0.864 | 0.909 | 0.850 | 0.967 | 0.932 | 0.872 | 0.991 |
| HERC6, SLC1A2, RETN, MMP8 | | | | | | | | | |
| RSAD2, OAS1, IFIT1, IFI44L, | 0.854 | 0.845 | 0.863 | 0.907 | 0.850 | 0.964 | 0.970 | 0.932 | 1 |
| SIGLEC1, SLC1A2, IL1R2, RETN | | | | | | | | | |
| RSAD2, OAS1, IFI44L, SIGLEC1, | 0.854 | 0.844 | 0.864 | 0.898 | 0.831 | 0.965 | 0.959 | 0.918 | 0.999 |
| HERC6, OLAH, RETN, MMP8 | | | | | | | | | |
| RSAD2, OAS1, IFI44L, SIGLEC1, | 0.854 | 0.844 | 0.864 | 0.901 | 0.839 | 0.962 | 0.959 | 0.917 | 1 |
| ISG15, IL1R2, OLAH, MMP8 | | | | | | | | | |
| OAS1, IFIT1, IFI44L, SIGLEC1, | 0.854 | 0.845 | 0.863 | 0.907 | 0.853 | 0.962 | 0.964 | 0.926 | 1 |
| ISG15, SLC1A2, IL1R2, MMP8 | | | | | | | | | |
| RSAD2, OAS1, IFIT1, IFI44L, | 0.854 | 0.845 | 0.863 | 0.905 | 0.849 | 0.962 | 0.970 | 0.934 | 1 |
| SIGLEC1, ISG15, SLC1A2, IL1R2 | | | | | | | | | |
| RSAD2, OAS1, IFIT1, IFI44L, | 0.854 | 0.844 | 0.864 | 0.911 | 0.853 | 0.969 | 0.922 | 0.845 | 0.999 |
| SLC1A2, OLAH, FAM20A, RETN | | | | | | | | | |
| RSAD2, IFIT1, SIGLEC1, ISG15, | 0.854 | 0.845 | 0.863 | 0.912 | 0.858 | 0.966 | 0.971 | 0.939 | 1 |
| SLC1A2, IL1R2, RETN, MMP8 | | | | | | | | | |
| RSAD2, IFIT1, IFI44L, SIGLEC1, | 0.854 | 0.845 | 0.863 | 0.911 | 0.856 | 0.966 | 0.970 | 0.937 | 1 |
| SLC1A2, IL1R2, RETN, MMP8 | | | | | | | | | |
| RSAD2, OAS1, IFIT1, SIGLEC1, | 0.854 | 0.844 | 0.863 | 0.908 | 0.853 | 0.964 | 0.970 | 0.933 | 1 |
| ISG15, SLC1A2, IL1R2, RETN | | | | | | | | | |
| RSAD2, OAS1, IFIT1, IFI44L, | 0.854 | 0.844 | 0.863 | 0.896 | 0.829 | 0.963 | 0.961 | 0.921 | 1 |
| SIGLEC1, ISG15, IL1R2, RETN | | | | | | | | | |
| RSAD2, OAS1, IFIT1, SIGLEC1, | 0.854 | 0.844 | 0.864 | 0.900 | 0.838 | 0.962 | 0.946 | 0.897 | 0.995 |
| ISG15, HERC6, OLAH, MMP8 | | | | | | | | | |
| RSAD2, OAS1, IFIT1, ISG15, | 0.854 | 0.844 | 0.864 | 0.907 | 0.851 | 0.964 | 0.936 | 0.855 | 1 |
| SLC1A2, IL1R2, FAM20A, RETN | | | | | | | | | |
| RSAD2, IFI27, OAS1, IFIT1, | 0.854 | 0.843 | 0.864 | 0.904 | 0.841 | 0.968 | 0.952 | 0.889 | 1 |
| IFI44L, ISG15, RETN, MMP8 | | | | | | | | | |
| OAS1, IFIT1, IFI44L, SIGLEC1, | 0.854 | 0.844 | 0.863 | 0.901 | 0.836 | 0.965 | 0.957 | 0.914 | 0.999 |
| ISG15, IL1R2, OLAH, RETN | | | | | | | | | |
| RSAD2, IFIT1, IFI44L, SIGLEC1, | 0.854 | 0.845 | 0.862 | 0.906 | 0.849 | 0.963 | 0.963 | 0.925 | 1 |
| ISG15, SLC1A2, IL1R2, RETN | | | | | | | | | |
| RSAD2, IFIT1, SIGLEC1, ISG15, | 0.854 | 0.844 | 0.864 | 0.902 | 0.840 | 0.964 | 0.968 | 0.935 | 1 |
| IL1R2, OLAH, RETN, MMP8 | | | | | | | | | |
| RSAD2, IFIT1, IFI44L, SIGLEC1, | 0.854 | 0.844 | 0.863 | 0.899 | 0.835 | 0.964 | 0.967 | 0.933 | 1 |
| ISG15, IL1R2, RETN, MMP8 | | | | | | | | | |
| RSAD2, OAS1, IFI44L, ISG15, | 0.854 | 0.843 | 0.864 | 0.901 | 0.836 | 0.965 | 0.941 | 0.870 | 1 |
| IL1R2, OLAH, FAM20A, RETN | | | | | | | | | |
| RSAD2, IFI27, OAS1, IFIT1, ISG15, | 0.854 | 0.843 | 0.864 | 0.907 | 0.849 | 0.965 | 0.932 | 0.849 | 1 |
| HERC6, SLC1A2, FAM20A | | | | | | | | | |
| RSAD2, IFI44L, ISG15, HERC6, | 0.853 | 0.843 | 0.864 | 0.910 | 0.849 | 0.970 | 0.927 | 0.847 | 1 |
| SLC1A2, FAM20A, RETN, MMP8 | | | | | | | | | |
| RSAD2, OAS1, IFIT1, IFI44L, | 0.853 | 0.843 | 0.864 | 0.900 | 0.834 | 0.965 | 0.941 | 0.870 | 1 |
| IL1R2, OLAH, FAM20A, RETN | | | | | | | | | |
| RSAD2, IFI27, OAS1, IFIT1, | 0.853 | 0.842 | 0.864 | 0.902 | 0.837 | 0.966 | 0.936 | 0.857 | 1 |
| IFI44L, ISG15, SLC1A2, FAM20A | | | | | | | | | |
| RSAD2, OAS1, IFIT1, IFI44L, | 0.853 | 0.844 | 0.863 | 0.900 | 0.838 | 0.961 | 0.960 | 0.919 | 1 |
| SIGLEC1, ISG15, IL1R2, MMP8 | | | | | | | | | |
| RSAD2, OAS1, IFIT1, IFI44L, | 0.853 | 0.843 | 0.863 | 0.907 | 0.851 | 0.964 | 0.926 | 0.839 | 1 |
| ISG15, SLC1A2, IL1R2, FAM20A | | | | | | | | | |
| RSAD2, OAS1, IFIT1, IFI44L, | 0.853 | 0.844 | 0.862 | 0.907 | 0.851 | 0.962 | 0.971 | 0.938 | 1 |
| SIGLEC1, SLC1A2, IL1R2, MMP8 | | | | | | | | | |
| RSAD2, IFI27, OAS1, IFIT1, ISG15, | 0.853 | 0.844 | 0.863 | 0.907 | 0.852 | 0.962 | 0.900 | 0.819 | 0.981 |
| HERC6, SLC1A2, MMP8 | | | | | | | | | |
| OAS1, IFIT1, IFI44L, ISG15, | 0.853 | 0.843 | 0.863 | 0.908 | 0.852 | 0.963 | 0.925 | 0.836 | 1 |
| SLC1A2, IL1R2, FAM20A, RETN | | | | | | | | | |
| RSAD2, OAS1, IFIT1, SIGLEC1, | 0.853 | 0.844 | 0.862 | 0.908 | 0.854 | 0.962 | 0.971 | 0.938 | 1 |
| ISG15, SLC1A2, IL1R2, MMP8 | | | | | | | | | |
| IFI27, OAS1, IFIT1, IFI44L, ISG15, | 0.853 | 0.844 | 0.862 | 0.907 | 0.853 | 0.961 | 0.897 | 0.817 | 0.976 |
| HERC6, SLC1A2, MMP8 | | | | | | | | | |
| OAS1, IFI44L, SIGLEC1, ISG15, | 0.853 | 0.843 | 0.863 | 0.903 | 0.841 | 0.965 | 0.963 | 0.925 | 1 |
| IL1R2, OLAH, RETN, MMP8 | | | | | | | | | |
| RSAD2, IFIT1, IFI44L, SIGLEC1, | 0.853 | 0.845 | 0.861 | 0.906 | 0.851 | 0.962 | 0.968 | 0.935 | 1 |
| ISG15, SLC1A2, IL1R2, MMP8 | | | | | | | | | |
| RSAD2, IFI27, OAS1, IFIT1, | 0.853 | 0.844 | 0.862 | 0.907 | 0.852 | 0.963 | 0.897 | 0.814 | 0.980 |
| IFI44L, HERC6, SLC1A2, MMP8 | | | | | | | | | |
| OAS1, IFI44L, SIGLEC1, ISG15, | 0.853 | 0.844 | 0.862 | 0.912 | 0.858 | 0.966 | 0.971 | 0.938 | 1 |
| SLC1A2, IL1R2, RETN, MMP8 | | | | | | | | | |
| RSAD2, IFI27, OAS1, IFIT1, | 0.853 | 0.843 | 0.863 | 0.904 | 0.840 | 0.968 | 0.945 | 0.874 | 1 |
| IFI44L, ISG15, SLC1A2, RETN | | | | | | | | | |
| RSAD2, OAS1, IFIT1, SIGLEC1, | 0.853 | 0.843 | 0.863 | 0.905 | 0.846 | 0.963 | 0.963 | 0.926 | 1 |
| ISG15, IL1R2, OLAH, MMP8 | | | | | | | | | |
| RSAD2, IFIT1, IFI44L, SIGLEC1, | 0.853 | 0.844 | 0.862 | 0.898 | 0.832 | 0.964 | 0.961 | 0.921 | 1 |
| ISG15, IL1R2, OLAH, RETN | | | | | | | | | |
| RSAD2, OAS1, SIGLEC1, ISG15, | 0.853 | 0.843 | 0.862 | 0.912 | 0.858 | 0.965 | 0.973 | 0.941 | 1 |
| SLC1A2, IL1R2, RETN, MMP8 | | | | | | | | | |
| RSAD2, IFI27, OAS1, IFI44L, | 0.853 | 0.843 | 0.862 | 0.906 | 0.851 | 0.961 | 0.901 | 0.821 | 0.981 |
| ISG15, HERC6, SLC1A2, MMP8 | | | | | | | | | |
| RSAD2, OAS1, IFIT1, SIGLEC1, | 0.853 | 0.843 | 0.863 | 0.898 | 0.831 | 0.964 | 0.961 | 0.920 | 1 |
| ISG15, IL1R2, OLAH, RETN | | | | | | | | | |
| RSAD2, OAS1, IFI44L, SIGLEC1, | 0.853 | 0.843 | 0.863 | 0.898 | 0.832 | 0.964 | 0.952 | 0.906 | 0.998 |
| ISG15, HERC6, OLAH, RETN | | | | | | | | | |
| OAS1, IFIT1, IFI44L, ISG15, IL1R2, | 0.853 | 0.843 | 0.863 | 0.900 | 0.835 | 0.966 | 0.942 | 0.872 | 1 |
| OLAH, FAM20A, RETN | | | | | | | | | |
| RSAD2, OAS1, IFIT1, SIGLEC1, | 0.853 | 0.843 | 0.863 | 0.901 | 0.838 | 0.965 | 0.971 | 0.938 | 1 |
| ISG15, IL1R2, RETN, MMP8 | | | | | | | | | |
| OAS1, IFI44L, ISG15, SLC1A2, | 0.853 | 0.842 | 0.863 | 0.912 | 0.859 | 0.965 | 0.925 | 0.832 | 1 |
| IL1R2, FAM20A, RETN, MMP8 | | | | | | | | | |
| OAS1, IFIT1, IFI44L, HERC6, | 0.853 | 0.842 | 0.863 | 0.907 | 0.848 | 0.966 | 0.936 | 0.878 | 0.993 |
| SLC1A2, OLAH, RETN, MMP8 | | | | | | | | | |
| IFI27, OAS1, IFIT1, IFI44L, ISG15, | 0.853 | 0.842 | 0.863 | 0.903 | 0.840 | 0.966 | 0.941 | 0.869 | 1 |
| SLC1A2, RETN, MMP8 | | | | | | | | | |
| RSAD2, OAS1, IFI44L, SLC1A2, | 0.853 | 0.842 | 0.863 | 0.912 | 0.859 | 0.965 | 0.926 | 0.835 | 1 |
| IL1R2, FAM20A, RETN, MMP8 | | | | | | | | | |
| RSAD2, OAS1, IFIT1, SLC1A2, | 0.853 | 0.842 | 0.863 | 0.906 | 0.846 | 0.967 | 0.942 | 0.872 | 1 |
| OLAH, FAM20A, RETN, MMP8 | | | | | | | | | |
| RSAD2, OAS1, IFI44L, SIGLEC1, | 0.853 | 0.842 | 0.863 | 0.900 | 0.836 | 0.964 | 0.970 | 0.937 | 1 |
| ISG15, IL1R2, RETN, MMP8 | | | | | | | | | |
| RSAD2, IFI27, IFIT1, IFI44L, | 0.853 | 0.843 | 0.862 | 0.903 | 0.844 | 0.962 | 0.925 | 0.855 | 0.995 |
| SIGLEC1, ISG15, SLC1A2, MMP8 | | | | | | | | | |
| RSAD2, OAS1, IFI44L, SIGLEC1, | 0.853 | 0.843 | 0.862 | 0.896 | 0.829 | 0.962 | 0.962 | 0.922 | 1 |
| ISG15, IL1R2, OLAH, RETN | | | | | | | | | |
| OAS1, IFIT1, IFI44L, ISG15, IL1R2, | 0.852 | 0.842 | 0.863 | 0.907 | 0.845 | 0.970 | 0.946 | 0.871 | 1 |
| FAM20A, RETN, MMP8 | | | | | | | | | |
| RSAD2, OAS1, ISG15, SLC1A2, | 0.852 | 0.843 | 0.862 | 0.913 | 0.860 | 0.967 | 0.928 | 0.837 | 1 |
| IL1R2, FAM20A, RETN, MMP8 | | | | | | | | | |
| RSAD2, OAS1, ISG15, SLC1A2, | 0.852 | 0.842 | 0.863 | 0.909 | 0.850 | 0.967 | 0.938 | 0.864 | 1 |
| OLAH, FAM20A, RETN, MMP8 | | | | | | | | | |
| OAS1, IFIT1, ISG15, SLC1A2, | 0.852 | 0.842 | 0.863 | 0.905 | 0.845 | 0.966 | 0.939 | 0.866 | 1 |
| OLAH, FAM20A, RETN, MMP8 | | | | | | | | | |
| OAS1, IFI44L, SIGLEC1, ISG15, | 0.852 | 0.842 | 0.863 | 0.905 | 0.844 | 0.965 | 0.952 | 0.903 | 1 |
| HERC6, SLC1A2, RETN, MMP8 | | | | | | | | | |
| RSAD2, IFI44L, SIGLEC1, ISG15, | 0.852 | 0.842 | 0.862 | 0.902 | 0.838 | 0.965 | 0.967 | 0.932 | 1 |
| IL1R2, OLAH, RETN, MMP8 | | | | | | | | | |
| RSAD2, IFIT1, ISG15, IL1R2, | 0.852 | 0.842 | 0.862 | 0.905 | 0.842 | 0.967 | 0.949 | 0.882 | 1 |
| OLAH, FAM20A, RETN, MMP8 | | | | | | | | | |
| RSAD2, OAS1, IFI44L, ISG15, | 0.852 | 0.842 | 0.863 | 0.908 | 0.848 | 0.969 | 0.941 | 0.865 | 1 |
| IL1R2, FAM20A, RETN, MMP8 | | | | | | | | | |
| RSAD2, OAS1, IFIT1, IFI44L, | 0.852 | 0.842 | 0.863 | 0.902 | 0.839 | 0.965 | 0.940 | 0.865 | 1 |
| ISG15, IL1R2, FAM20A, RETN | | | | | | | | | |
| RSAD2, OAS1, IFIT1, IFI44L, | 0.852 | 0.842 | 0.862 | 0.907 | 0.851 | 0.963 | 0.927 | 0.841 | 1 |
| SLC1A2, IL1R2, FAM20A, RETN | | | | | | | | | |
| RSAD2, IFI44L, ISG15, SLC1A2, | 0.852 | 0.842 | 0.862 | 0.912 | 0.860 | 0.965 | 0.926 | 0.835 | 1 |
| IL1R2, FAM20A, RETN, MMP8 | | | | | | | | | |
| OAS1, IFIT1, IFI44L, ISG15, | 0.852 | 0.842 | 0.863 | 0.910 | 0.854 | 0.967 | 0.934 | 0.872 | 0.996 |
| HERC6, SLC1A2, OLAH, RETN | | | | | | | | | |
| RSAD2, IFIT1, IFI44L, ISG15, | 0.852 | 0.842 | 0.862 | 0.907 | 0.851 | 0.962 | 0.926 | 0.839 | 1 |
| SLC1A2, IL1R2, FAM20A, RETN | | | | | | | | | |
| RSAD2, OAS1, IFI44L, SIGLEC1, | 0.852 | 0.843 | 0.861 | 0.911 | 0.857 | 0.966 | 0.974 | 0.944 | 1 |
| SLC1A2, IL1R2, RETN, MMP8 | | | | | | | | | |
| RSAD2, IFI27, OAS1, IFI44L, | 0.852 | 0.842 | 0.862 | 0.905 | 0.842 | 0.967 | 0.939 | 0.868 | 1 |
| ISG15, SLC1A2, RETN, MMP8 | | | | | | | | | |
| RSAD2, IFIT1, ISG15, SLC1A2, | 0.852 | 0.842 | 0.862 | 0.905 | 0.845 | 0.966 | 0.941 | 0.871 | 1 |
| OLAH, FAM20A, RETN, MMP8 | | | | | | | | | |
| RSAD2, OAS1, IFIT1, IFI44L, | 0.852 | 0.842 | 0.862 | 0.900 | 0.838 | 0.961 | 0.942 | 0.892 | 0.993 |
| SIGLEC1, ISG15, HERC6, OLAH | | | | | | | | | |
| RSAD2, IFIT1, IFI44L, SIGLEC1, | 0.852 | 0.842 | 0.862 | 0.900 | 0.838 | 0.962 | 0.942 | 0.892 | 0.993 |
| ISG15, HERC6, OLAH, MMP8 | | | | | | | | | |
| RSAD2, OAS1, IFI44L, SIGLEC1, | 0.852 | 0.842 | 0.862 | 0.905 | 0.844 | 0.965 | 0.952 | 0.902 | 1 |
| ISG15, HERC6, SLC1A2, RETN | | | | | | | | | |
| RSAD2, IFI44L, SIGLEC1, ISG15, | 0.852 | 0.843 | 0.861 | 0.911 | 0.856 | 0.966 | 0.970 | 0.937 | 1 |
| SLC1A2, IL1R2, RETN, MMP8 | | | | | | | | | |
| RSAD2, OAS1, IFIT1, ISG15, | 0.852 | 0.841 | 0.862 | 0.904 | 0.841 | 0.966 | 0.935 | 0.858 | 1 |
| HERC6, FAM20A, RETN, MMP8 | | | | | | | | | |
| RSAD2, IFI27, OAS1, IFIT1, ISG15, | 0.852 | 0.841 | 0.862 | 0.902 | 0.839 | 0.966 | 0.940 | 0.866 | 1 |
| SLC1A2, RETN, MMP8 | | | | | | | | | |
| RSAD2, IFI27, OAS1, IFIT1, | 0.852 | 0.842 | 0.862 | 0.904 | 0.841 | 0.968 | 0.940 | 0.867 | 1 |
| IFI44L, SLC1A2, RETN, MMP8 | | | | | | | | | |
| RSAD2, OAS1, IFIT1, IFI44L, | 0.852 | 0.841 | 0.862 | 0.897 | 0.829 | 0.964 | 0.955 | 0.912 | 0.999 |
| SIGLEC1, OLAH, RETN, MMP8 | | | | | | | | | |
| RSAD2, OAS1, ISG15, HERC6, | 0.852 | 0.841 | 0.862 | 0.901 | 0.840 | 0.963 | 0.959 | 0.918 | 1.000 |
| IL1R2, OLAH, RETN, MMP8 | | | | | | | | | |
| RSAD2, IFI44L, SIGLEC1, ISG15, | 0.852 | 0.841 | 0.862 | 0.900 | 0.834 | 0.966 | 0.958 | 0.916 | 0.999 |
| HERC6, OLAH, RETN, MMP8 | | | | | | | | | |
| RSAD2, IFIT1, IFI44L, IL1R2, | 0.852 | 0.841 | 0.862 | 0.905 | 0.843 | 0.968 | 0.951 | 0.889 | 1 |
| OLAH, FAM20A, RETN, MMP8 | | | | | | | | | |
| OAS1, IFI44L, ISG15, HERC6, | 0.852 | 0.840 | 0.863 | 0.906 | 0.844 | 0.968 | 0.932 | 0.850 | 1 |
| SLC1A2, FAM20A, RETN, MMP8 | | | | | | | | | |
| RSAD2, IFIT1, IFI44L, SIGLEC1, | 0.852 | 0.842 | 0.861 | 0.899 | 0.834 | 0.965 | 0.929 | 0.872 | 0.987 |
| ISG15, HERC6, RETN, MMP8 | | | | | | | | | |
| RSAD2, OAS1, IFI44L, SIGLEC1, | 0.851 | 0.842 | 0.861 | 0.907 | 0.851 | 0.962 | 0.973 | 0.943 | 1 |
| ISG15, SLC1A2, IL1R2, MMP8 | | | | | | | | | |
| RSAD2, IFIT1, IFI44L, SIGLEC1, | 0.851 | 0.842 | 0.860 | 0.907 | 0.849 | 0.965 | 0.927 | 0.868 | 0.987 |
| ISG15, HERC6, SLC1A2, RETN | | | | | | | | | |
| OAS1, IFIT1, IFI44L, SIGLEC1, | 0.851 | 0.841 | 0.862 | 0.898 | 0.831 | 0.965 | 0.957 | 0.913 | 1.000 |
| ISG15, OLAH, RETN, MMP8 | | | | | | | | | |
| OAS1, IFI44L, ISG15, HERC6, | 0.851 | 0.841 | 0.862 | 0.910 | 0.852 | 0.967 | 0.947 | 0.894 | 0.999 |
| SLC1A2, OLAH, RETN, MMP8 | | | | | | | | | |
| RSAD2, OAS1, IFIT1, ISG15, | 0.851 | 0.841 | 0.862 | 0.902 | 0.841 | 0.964 | 0.936 | 0.855 | 1 |
| HERC6, SLC1A2, FAM20A, RETN | | | | | | | | | |
| OAS1, IFIT1, HERC6, SLC1A2, | 0.851 | 0.841 | 0.861 | 0.905 | 0.848 | 0.963 | 0.959 | 0.917 | 1 |
| IL1R2, OLAH, RETN, MMP8 | | | | | | | | | |
| RSAD2, OAS1, IFI44L, ISG15, | 0.851 | 0.840 | 0.862 | 0.908 | 0.852 | 0.964 | 0.924 | 0.835 | 1 |
| SLC1A2, IL1R2, FAM20A, RETN | | | | | | | | | |
| RSAD2, OAS1, IFIT1, ISG15, | 0.851 | 0.840 | 0.861 | 0.908 | 0.850 | 0.967 | 0.935 | 0.861 | 1 |
| SLC1A2, OLAH, FAM20A, RETN | | | | | | | | | |
| RSAD2, OAS1, IFIT1, IFI44L, | 0.851 | 0.840 | 0.862 | 0.898 | 0.830 | 0.966 | 0.945 | 0.885 | 1 |
| ISG15, OLAH, FAM20A, MMP8 | | | | | | | | | |
| RSAD2, OAS1, IFIT1, ISG15, | 0.851 | 0.841 | 0.861 | 0.897 | 0.832 | 0.961 | 0.960 | 0.918 | 1 |
| HERC6, IL1R2, RETN, MMP8 | | | | | | | | | |
| OAS1, IFI44L, HERC6, SLC1A2, | 0.851 | 0.840 | 0.861 | 0.909 | 0.852 | 0.967 | 0.945 | 0.893 | 0.996 |
| IL1R2, OLAH, RETN, MMP8 | | | | | | | | | |
| RSAD2, OAS1, IFIT1, IFI44L, | 0.851 | 0.840 | 0.861 | 0.908 | 0.850 | 0.966 | 0.926 | 0.859 | 0.994 |
| HERC6, SLC1A2, OLAH, RETN | | | | | | | | | |
| RSAD2, OAS1, ISG15, HERC6, | 0.851 | 0.840 | 0.861 | 0.904 | 0.842 | 0.965 | 0.935 | 0.855 | 1 |
| SLC1A2, FAM20A, RETN, MMP8 | | | | | | | | | |
| IFIT1, IFI44L, ISG15, HERC6, | 0.851 | 0.840 | 0.861 | 0.907 | 0.846 | 0.967 | 0.935 | 0.854 | 1 |
| SLC1A2, FAM20A, RETN, MMP8 | | | | | | | | | |
| IFIT1, IFI44L, HERC6, SLC1A2, | 0.850 | 0.840 | 0.861 | 0.908 | 0.849 | 0.966 | 0.948 | 0.899 | 0.997 |
| IL1R2, OLAH, RETN, MMP8 | | | | | | | | | |
| RSAD2, IFIT1, SIGLEC1, ISG15, | 0.850 | 0.841 | 0.860 | 0.905 | 0.846 | 0.964 | 0.934 | 0.878 | 0.990 |
| HERC6, SLC1A2, RETN, MMP8 | | | | | | | | | |
| OAS1, IFIT1, ISG15, HERC6, | 0.850 | 0.840 | 0.861 | 0.900 | 0.838 | 0.963 | 0.962 | 0.923 | 1 |
| IL1R2, OLAH, RETN, MMP8 | | | | | | | | | |
| RSAD2, OAS1, HERC6, SLC1A2, | 0.850 | 0.840 | 0.860 | 0.903 | 0.844 | 0.962 | 0.942 | 0.885 | 1 |
| IL1R2, OLAH, RETN, MMP8 | | | | | | | | | |
| OAS1, IFI44L, ISG15, HERC6, | 0.850 | 0.839 | 0.861 | 0.908 | 0.851 | 0.965 | 0.942 | 0.884 | 1 |
| SLC1A2, IL1R2, OLAH, RETN | | | | | | | | | |
| IFIT1, IFI44L, ISG15, HERC6, | 0.850 | 0.840 | 0.860 | 0.908 | 0.850 | 0.966 | 0.941 | 0.889 | 0.993 |
| SLC1A2, OLAH, RETN, MMP8 | | | | | | | | | |
| RSAD2, OAS1, IFIT1, HERC6, | 0.850 | 0.840 | 0.860 | 0.904 | 0.846 | 0.961 | 0.953 | 0.908 | 0.999 |
| SLC1A2, IL1R2, OLAH, MMP8 | | | | | | | | | |
| IFI44L, ISG15, HERC6, SLC1A2, | 0.850 | 0.840 | 0.860 | 0.909 | 0.851 | 0.967 | 0.951 | 0.904 | 0.998 |
| IL1R2, OLAH, RETN, MMP8 | | | | | | | | | |
| RSAD2, OAS1, IFI44L, HERC6, | 0.850 | 0.839 | 0.861 | 0.905 | 0.848 | 0.963 | 0.936 | 0.874 | 0.998 |
| SLC1A2, IL1R2, OLAH, RETN | | | | | | | | | |
| RSAD2, IFI44L, HERC6, SLC1A2, | 0.850 | 0.840 | 0.860 | 0.910 | 0.853 | 0.967 | 0.941 | 0.889 | 0.994 |
| IL1R2, OLAH, RETN, MMP8 | | | | | | | | | |

Performance obtained for the identification of the viral or bacterial nature of the infection on the basis of the nine-gene combinations:

TABLE 13

| | AUC | | | AUC | CI 95% | | AUC | CI 95% | |
|---|---|---|---|---|---|---|---|---|---|
| Model | model | CI 95% | | Train | Train | | Test | Test | |
| IFI27, OAS1, IFIT1, SIGLEC1, HERC6, | 0.895 | 0.886 | 0.904 | 0.938 | 0.889 | 0.987 | 0.951 | 0.888 | 1 |
| IL1R2, OLAH, FAM20A, MMP8 | | | | | | | | | |
| IFI27, IFIT1, IFI44L, SIGLEC1, HERC6, | 0.894 | 0.885 | 0.902 | 0.937 | 0.888 | 0.986 | 0.955 | 0.894 | 1 |
| IL1R2, OLAH, FAM20A, MMP8 | | | | | | | | | |
| IFI27, IFIT1, SIGLEC1, ISG15, HERC6, | 0.894 | 0.885 | 0.903 | 0.937 | 0.887 | 0.986 | 0.953 | 0.891 | 1 |
| IL1R2, OLAH, FAM20A, MMP8 | | | | | | | | | |
| IFI27, IFI44L, SIGLEC1, ISG15, HERC6, | 0.894 | 0.885 | 0.903 | 0.936 | 0.886 | 0.986 | 0.948 | 0.884 | 1 |
| IL1R2, OLAH, FAM20A, MMP8 | | | | | | | | | |
| RSAD2, IFI27, IFIT1, SIGLEC1, HERC6, | 0.893 | 0.885 | 0.902 | 0.937 | 0.888 | 0.987 | 0.951 | 0.890 | 1 |
| IL1R2, OLAH, FAM20A, MMP8 | | | | | | | | | |
| IFI27, OAS1, IFIT1, SIGLEC1, HERC6, | 0.893 | 0.884 | 0.903 | 0.937 | 0.884 | 0.990 | 0.949 | 0.887 | 1 |
| OLAH, FAM20A, RETN, MMP8 | | | | | | | | | |
| RSAD2, IFI27, OAS1, SIGLEC1, HERC6, | 0.893 | 0.883 | 0.902 | 0.937 | 0.887 | 0.987 | 0.951 | 0.891 | 1 |
| IL1R2, OLAH, FAM20A, MMP8 | | | | | | | | | |
| IFI27, OAS1, SIGLEC1, ISG15, HERC6, | 0.893 | 0.883 | 0.902 | 0.935 | 0.884 | 0.987 | 0.950 | 0.886 | 1 |
| IL1R2, OLAH, FAM20A, MMP8 | | | | | | | | | |
| IFI27, IFIT1, IFI44L, SIGLEC1, ISG15, | 0.893 | 0.884 | 0.901 | 0.937 | 0.888 | 0.986 | 0.951 | 0.890 | 1 |
| IL1R2, OLAH, FAM20A, MMP8 | | | | | | | | | |
| RSAD2, IFI27, OAS1, IFIT1, SIGLEC1, | 0.892 | 0.883 | 0.902 | 0.935 | 0.883 | 0.986 | 0.946 | 0.884 | 1 |
| HERC6, OLAH, FAM20A, MMP8 | | | | | | | | | |
| IFI27, OAS1, IFIT1, SIGLEC1, HERC6, | 0.892 | 0.883 | 0.901 | 0.937 | 0.887 | 0.988 | 0.951 | 0.883 | 1 |
| IL1R2, FAM20A, RETN, MMP8 | | | | | | | | | |
| IFI27, OAS1, IFIT1, IFI44L, SIGLEC1, | 0.892 | 0.883 | 0.902 | 0.934 | 0.882 | 0.986 | 0.946 | 0.883 | 1 |
| HERC6, OLAH, FAM20A, MMP8 | | | | | | | | | |
| IFI27, OAS1, IFIT1, SIGLEC1, ISG15, | 0.892 | 0.883 | 0.901 | 0.936 | 0.886 | 0.987 | 0.951 | 0.888 | 1 |
| IL1R2, OLAH, FAM20A, MMP8 | | | | | | | | | |
| IFI27, OAS1, IFIT1, IFI44L, SIGLEC1, | 0.892 | 0.883 | 0.901 | 0.934 | 0.884 | 0.983 | 0.948 | 0.881 | 1 |
| HERC6, IL1R2, FAM20A, MMP8 | | | | | | | | | |
| RSAD2, IFI27, OAS1, IFIT1, SIGLEC1, | 0.892 | 0.883 | 0.901 | 0.935 | 0.884 | 0.986 | 0.954 | 0.897 | 1 |
| IL1R2, OLAH, FAM20A, MMP8 | | | | | | | | | |
| IFI27, OAS1, IFIT1, IFI44L, SIGLEC1, | 0.892 | 0.883 | 0.901 | 0.935 | 0.885 | 0.985 | 0.955 | 0.898 | 1 |
| IL1R2, OLAH, FAM20A, MMP8 | | | | | | | | | |
| IFI27, OAS1, IFIT1, SIGLEC1, ISG15, | 0.892 | 0.882 | 0.901 | 0.931 | 0.877 | 0.986 | 0.946 | 0.884 | 1 |
| HERC6, OLAH, FAM20A, MMP8 | | | | | | | | | |
| RSAD2, IFI27, SIGLEC1, ISG15, HERC6, | 0.892 | 0.882 | 0.901 | 0.937 | 0.886 | 0.987 | 0.951 | 0.891 | 1 |
| IL1R2, OLAH, FAM20A, MMP8 | | | | | | | | | |
| IFI27, OAS1, IFI44L, SIGLEC1, HERC6, | 0.891 | 0.882 | 0.901 | 0.937 | 0.888 | 0.986 | 0.950 | 0.889 | 1 |
| IL1R2, OLAH, FAM20A, MMP8 | | | | | | | | | |
| RSAD2, IFI27, IFI44L, SIGLEC1, | 0.891 | 0.882 | 0.900 | 0.936 | 0.887 | 0.985 | 0.951 | 0.890 | 1 |
| HERC6, IL1R2, OLAH, FAM20A, MMP8 | | | | | | | | | |
| IFI27, IFIT1, IFI44L, SIGLEC1, ISG15, | 0.891 | 0.882 | 0.901 | 0.932 | 0.878 | 0.986 | 0.948 | 0.886 | 1 |
| HERC6, OLAH, FAM20A, MMP8 | | | | | | | | | |
| IFI27, OAS1, SIGLEC1, HERC6, IL1R2, | 0.891 | 0.881 | 0.901 | 0.935 | 0.881 | 0.988 | 0.949 | 0.885 | 1 |
| OLAH, FAM20A, RETN, MMP8 | | | | | | | | | |
| RSAD2, IFI27, IFIT1, IFI44L, SIGLEC1, | 0.891 | 0.882 | 0.900 | 0.934 | 0.882 | 0.985 | 0.948 | 0.887 | 1 |
| HERC6, OLAH, FAM20A, MMP8 | | | | | | | | | |
| IFI27, OAS1, IFI44L, SIGLEC1, ISG15, | 0.891 | 0.881 | 0.900 | 0.935 | 0.885 | 0.985 | 0.952 | 0.892 | 1 |
| IL1R2, OLAH, FAM20A, MMP8 | | | | | | | | | |
| RSAD2, IFI27, OAS1, SIGLEC1, HERC6, | 0.891 | 0.881 | 0.900 | 0.935 | 0.880 | 0.989 | 0.948 | 0.888 | 1 |
| OLAH, FAM20A, RETN, MMP8 | | | | | | | | | |
| RSAD2, IFI27, IFIT1, SIGLEC1, ISG15, | 0.891 | 0.882 | 0.899 | 0.937 | 0.886 | 0.987 | 0.952 | 0.891 | 1 |
| IL1R2, OLAH, FAM20A, MMP8 | | | | | | | | | |
| IFI27, IFIT1, IFI44L, SIGLEC1, ISG15, | 0.891 | 0.882 | 0.899 | 0.932 | 0.882 | 0.983 | 0.953 | 0.886 | 1 |
| HERC6, IL1R2, FAM20A, MMP8 | | | | | | | | | |
| IFI27, IFIT1, IFI44L, SIGLEC1, HERC6, | 0.890 | 0.882 | 0.899 | 0.937 | 0.887 | 0.987 | 0.951 | 0.882 | 1 |
| IL1R2, FAM20A, RETN, MMP8 | | | | | | | | | |
| RSAD2, IFI27, IFIT1, IFI44L, SIGLEC1, | 0.890 | 0.882 | 0.899 | 0.936 | 0.886 | 0.986 | 0.954 | 0.896 | 1 |
| IL1R2, OLAH, FAM20A, MMP8 | | | | | | | | | |
| RSAD2, IFI27, SIGLEC1, HERC6, IL1R2, | 0.890 | 0.881 | 0.900 | 0.936 | 0.883 | 0.988 | 0.950 | 0.887 | 1 |
| OLAH, FAM20A, RETN, MMP8 | | | | | | | | | |
| IFI27, OAS1, SIGLEC1, ISG15, HERC6, | 0.890 | 0.880 | 0.900 | 0.934 | 0.879 | 0.989 | 0.948 | 0.886 | 1 |
| OLAH, FAM20A, RETN, MMP8 | | | | | | | | | |
| RSAD2, IFI27, IFIT1, SIGLEC1, ISG15, | 0.890 | 0.881 | 0.900 | 0.934 | 0.882 | 0.987 | 0.945 | 0.883 | 1 |
| HERC6, OLAH, FAM20A, MMP8 | | | | | | | | | |
| IFI27, IFIT1, SIGLEC1, HERC6, IL1R2, | 0.890 | 0.881 | 0.900 | 0.935 | 0.883 | 0.988 | 0.957 | 0.897 | 1 |
| OLAH, FAM20A, RETN, MMP8 | | | | | | | | | |
| IFI27, OAS1, IFIT1, IFI44L, SIGLEC1, | 0.890 | 0.882 | 0.899 | 0.935 | 0.885 | 0.985 | 0.942 | 0.877 | 1 |
| HERC6, IL1R2, OLAH, FAM20A | | | | | | | | | |
| RSAD2, IFI27, OAS1, IFIT1, SIGLEC1, | 0.890 | 0.881 | 0.899 | 0.934 | 0.884 | 0.983 | 0.951 | 0.885 | 1 |
| HERC6, IL1R2, FAM20A, MMP8 | | | | | | | | | |
| IFI27, IFIT1, IFI44L, SIGLEC1, ISG15, | 0.890 | 0.881 | 0.899 | 0.932 | 0.880 | 0.985 | 0.943 | 0.879 | 1 |
| HERC6, IL1R2, OLAH, FAM20A | | | | | | | | | |
| IFI27, OAS1, IFIT1, IFI44L, SIGLEC1, | 0.890 | 0.881 | 0.899 | 0.931 | 0.878 | 0.985 | 0.938 | 0.873 | 1 |
| HERC6, OLAH, FAM20A, RETN | | | | | | | | | |
| RSAD2, IFI27, IFIT1, IFI44L, SIGLEC1, | 0.890 | 0.882 | 0.898 | 0.934 | 0.885 | 0.984 | 0.949 | 0.880 | 1 |
| HERC6, IL1R2, FAM20A, MMP8 | | | | | | | | | |
| IFI27, IFIT1, SIGLEC1, ISG15, IL1R2, | 0.890 | 0.880 | 0.899 | 0.934 | 0.881 | 0.987 | 0.957 | 0.896 | 1 |
| OLAH, FAM20A, RETN, MMP8 | | | | | | | | | |
| RSAD2, IFI27, OAS1, SIGLEC1, ISG15, | 0.890 | 0.880 | 0.900 | 0.934 | 0.881 | 0.988 | 0.949 | 0.889 | 1 |
| HERC6, OLAH, FAM20A, MMP8 | | | | | | | | | |
| IFI27, IFIT1, IFI44L, SIGLEC1, HERC6, | 0.890 | 0.880 | 0.899 | 0.934 | 0.880 | 0.987 | 0.955 | 0.895 | 1 |
| OLAH, FAM20A, RETN, MMP8 | | | | | | | | | |
| RSAD2, IFI27, IFIT1, SIGLEC1, HERC6, | 0.890 | 0.881 | 0.898 | 0.937 | 0.887 | 0.988 | 0.950 | 0.881 | 1 |
| IL1R2, FAM20A, RETN, MMP8 | | | | | | | | | |
| RSAD2, IFI27, OAS1, IFIT1, IFI44L, | 0.890 | 0.881 | 0.898 | 0.931 | 0.879 | 0.984 | 0.940 | 0.876 | 1 |
| SIGLEC1, HERC6, OLAH, FAM20A | | | | | | | | | |
| RSAD2, IFI27, OAS1, IFIT1, SIGLEC1, | 0.890 | 0.881 | 0.898 | 0.931 | 0.880 | 0.983 | 0.941 | 0.878 | 1 |
| HERC6, IL1R2, OLAH, FAM20A | | | | | | | | | |
| IFI27, OAS1, IFIT1, SIGLEC1, ISG15, | 0.890 | 0.880 | 0.899 | 0.932 | 0.879 | 0.985 | 0.937 | 0.872 | 1 |
| HERC6, OLAH, FAM20A, RETN | | | | | | | | | |
| IFI27, IFIT1, IFI44L, SIGLEC1, ISG15, | 0.889 | 0.881 | 0.898 | 0.931 | 0.876 | 0.986 | 0.941 | 0.878 | 1 |
| HERC6, OLAH, FAM20A, RETN | | | | | | | | | |
| RSAD2, IFI27, OAS1, SIGLEC1, ISG15, | 0.889 | 0.880 | 0.899 | 0.936 | 0.885 | 0.987 | 0.952 | 0.892 | 1 |
| IL1R2, OLAH, FAM20A, MMP8 | | | | | | | | | |
| IFI27, OAS1, SIGLEC1, ISG15, IL1R2, | 0.889 | 0.880 | 0.899 | 0.933 | 0.880 | 0.987 | 0.951 | 0.889 | 1 |
| OLAH, FAM20A, RETN, MMP8 | | | | | | | | | |
| IFI27, OAS1, IFIT1, SIGLEC1, ISG15, | 0.889 | 0.880 | 0.898 | 0.934 | 0.885 | 0.983 | 0.952 | 0.885 | 1 |
| HERC6, IL1R2, FAM20A, MMP8 | | | | | | | | | |
| IFI27, IFIT1, SIGLEC1, ISG15, HERC6, | 0.889 | 0.879 | 0.900 | 0.933 | 0.879 | 0.988 | 0.954 | 0.894 | 1 |
| OLAH, FAM20A, RETN, MMP8 | | | | | | | | | |
| IFI27, IFI44L, SIGLEC1, ISG15, IL1R2, | 0.889 | 0.880 | 0.899 | 0.936 | 0.884 | 0.988 | 0.950 | 0.885 | 1 |
| OLAH, FAM20A, RETN, MMP8 | | | | | | | | | |
| RSAD2, IFI27, IFIT1, SIGLEC1, ISG15, | 0.889 | 0.881 | 0.898 | 0.934 | 0.884 | 0.984 | 0.953 | 0.886 | 1 |
| HERC6, IL1R2, FAM20A, MMP8 | | | | | | | | | |
| IFI27, IFIT1, SIGLEC1, ISG15, HERC6, | 0.889 | 0.880 | 0.898 | 0.935 | 0.883 | 0.987 | 0.953 | 0.885 | 1 |
| IL1R2, FAM20A, RETN, MMP8 | | | | | | | | | |
| RSAD2, IFI27, IFIT1, SIGLEC1, HERC6, | 0.889 | 0.879 | 0.899 | 0.937 | 0.884 | 0.990 | 0.947 | 0.882 | 1 |
| OLAH, FAM20A, RETN, MMP8 | | | | | | | | | |
| IFI27, OAS1, IFIT1, IFI44L, SIGLEC1, | 0.889 | 0.880 | 0.899 | 0.932 | 0.880 | 0.985 | 0.939 | 0.875 | 1 |
| ISG15, HERC6, OLAH, FAM20A | | | | | | | | | |
| IFI27, OAS1, IFIT1, SIGLEC1, ISG15, | 0.889 | 0.880 | 0.898 | 0.934 | 0.883 | 0.985 | 0.937 | 0.871 | 1 |
| HERC6, IL1R2, OLAH, FAM20A | | | | | | | | | |
| RSAD2, IFI27, SIGLEC1, ISG15, HERC6, | 0.889 | 0.879 | 0.899 | 0.936 | 0.882 | 0.990 | 0.948 | 0.887 | 1 |
| OLAH, FAM20A, RETN, MMP8 | | | | | | | | | |
| IFI27, OAS1, IFIT1, IFI44L, SIGLEC1, | 0.889 | 0.879 | 0.899 | 0.929 | 0.872 | 0.986 | 0.949 | 0.891 | 1 |
| ISG15, OLAH, FAM20A, MMP8 | | | | | | | | | |
| IFI27, OAS1, IFIT1, SIGLEC1, HERC6, | 0.889 | 0.880 | 0.898 | 0.932 | 0.879 | 0.985 | 0.939 | 0.874 | 1 |
| IL1R2, OLAH, FAM20A, RETN | | | | | | | | | |
| IFI27, OAS1, IFI44L, SIGLEC1, HERC6, | 0.889 | 0.879 | 0.899 | 0.937 | 0.887 | 0.988 | 0.948 | 0.878 | 1 |
| IL1R2, FAM20A, RETN, MMP8 | | | | | | | | | |
| RSAD2, IFI27, OAS1, IFI44L, SIGLEC1, | 0.889 | 0.879 | 0.898 | 0.933 | 0.881 | 0.985 | 0.948 | 0.887 | 1 |
| HERC6, OLAH, FAM20A, MMP8 | | | | | | | | | |
| RSAD2, IFI27, IFIT1, IFI44L, SIGLEC1, | 0.889 | 0.880 | 0.897 | 0.934 | 0.882 | 0.986 | 0.937 | 0.871 | 1 |
| HERC6, IL1R2, OLAH, FAM20A | | | | | | | | | |
| IFI27, OAS1, IFIT1, SIGLEC1, IL1R2, | 0.889 | 0.879 | 0.898 | 0.934 | 0.879 | 0.988 | 0.955 | 0.896 | 1 |
| OLAH, FAM20A, RETN, MMP8 | | | | | | | | | |
| RSAD2, IFI27, OAS1, IFI44L, SIGLEC1, | 0.889 | 0.879 | 0.898 | 0.935 | 0.884 | 0.985 | 0.953 | 0.895 | 1 |
| IL1R2, OLAH, FAM20A, MMP8 | | | | | | | | | |
| RSAD2, IFI27, IFI44L, SIGLEC1, ISG15, | 0.888 | 0.879 | 0.898 | 0.936 | 0.886 | 0.985 | 0.952 | 0.893 | 1 |
| IL1R2, OLAH, FAM20A, MMP8 | | | | | | | | | |
| IFI27, OAS1, SIGLEC1, ISG15, HERC6, | 0.888 | 0.879 | 0.898 | 0.934 | 0.882 | 0.986 | 0.951 | 0.883 | 1 |
| IL1R2, FAM20A, RETN, MMP8 | | | | | | | | | |
| IFI27, OAS1, IFI44L, SIGLEC1, ISG15, | 0.888 | 0.879 | 0.897 | 0.935 | 0.886 | 0.984 | 0.949 | 0.880 | 1 |
| HERC6, IL1R2, FAM20A, MMP8 | | | | | | | | | |
| RSAD2, IFI27, OAS1, IFIT1, SIGLEC1, | 0.888 | 0.879 | 0.897 | 0.933 | 0.880 | 0.985 | 0.939 | 0.875 | 1 |
| ISG15, HERC6, OLAH, FAM20A | | | | | | | | | |
| IFI27, IFI44L, SIGLEC1, ISG15, | 0.888 | 0.880 | 0.897 | 0.942 | 0.899 | 0.985 | 0.946 | 0.876 | 1 |
| SLC1A2, IL1R2, OLAH, FAM20A, MMP8 | | | | | | | | | |
| RSAD2, IFI27, OAS1, IFI44L, SIGLEC1, | 0.888 | 0.879 | 0.897 | 0.934 | 0.885 | 0.984 | 0.948 | 0.881 | 1 |
| HERC6, IL1R2, FAM20A, MMP8 | | | | | | | | | |
| IFI27, IFIT1, SIGLEC1, ISG15, SLC1A2, | 0.888 | 0.879 | 0.897 | 0.944 | 0.901 | 0.986 | 0.947 | 0.879 | 1 |
| IL1R2, OLAH, FAM20A, MMP8 | | | | | | | | | |
| IFI27, OAS1, IFIT1, SIGLEC1, ISG15, | 0.888 | 0.879 | 0.898 | 0.932 | 0.879 | 0.986 | 0.942 | 0.873 | 1 |
| HERC6, FAM20A, RETN, MMP8 | | | | | | | | | |
| RSAD2, IFI27, OAS1, SIGLEC1, IL1R2, | 0.888 | 0.878 | 0.898 | 0.934 | 0.881 | 0.988 | 0.951 | 0.890 | 1 |
| OLAH, FAM20A, RETN, MMP8 | | | | | | | | | |
| RSAD2, IFI27, OAS1, IFIT1, IFI44L, | 0.888 | 0.879 | 0.897 | 0.928 | 0.871 | 0.985 | 0.954 | 0.899 | 1 |
| SIGLEC1, OLAH, FAM20A, MMP8 | | | | | | | | | |
| IFI27, OAS1, IFIT1, IFI44L, SIGLEC1, | 0.888 | 0.879 | 0.897 | 0.931 | 0.878 | 0.984 | 0.936 | 0.864 | 1 |
| ISG15, HERC6, FAM20A, RETN | | | | | | | | | |
| RSAD2, IFI27, OAS1, IFIT1, SIGLEC1, | 0.888 | 0.879 | 0.897 | 0.930 | 0.873 | 0.987 | 0.949 | 0.891 | 1 |
| ISG15, OLAH, FAM20A, MMP8 | | | | | | | | | |
| IFI27, OAS1, IFIT1, IFI44L, SIGLEC1, | 0.888 | 0.879 | 0.897 | 0.934 | 0.882 | 0.985 | 0.954 | 0.893 | 1 |
| IL1R2, FAM20A, RETN, MMP8 | | | | | | | | | |
| RSAD2, IFI27, OAS1, SIGLEC1, HERC6, | 0.888 | 0.878 | 0.898 | 0.934 | 0.882 | 0.986 | 0.950 | 0.882 | 1 |
| IL1R2, FAM20A, RETN, MMP8 | | | | | | | | | |
| IFI27, OAS1, IFI44L, SIGLEC1, ISG15, | 0.888 | 0.878 | 0.898 | 0.930 | 0.875 | 0.985 | 0.946 | 0.883 | 1 |
| HERC6, OLAH, FAM20A, MMP8 | | | | | | | | | |
| IFI27, IFIT1, IFI44L, SIGLEC1, IL1R2, | 0.888 | 0.878 | 0.898 | 0.935 | 0.882 | 0.988 | 0.955 | 0.895 | 1 |
| OLAH, FAM20A, RETN, MMP8 | | | | | | | | | |
| IFI27, OAS1, IFIT1, IFI44L, SIGLEC1, | 0.888 | 0.879 | 0.897 | 0.934 | 0.884 | 0.984 | 0.945 | 0.878 | 1 |
| HERC6, IL1R2, FAM20A, RETN | | | | | | | | | |
| RSAD2, IFI27, SIGLEC1, ISG15, HERC6, | 0.888 | 0.878 | 0.897 | 0.936 | 0.884 | 0.988 | 0.949 | 0.879 | 1 |
| IL1R2, FAM20A, RETN, MMP8 | | | | | | | | | |
| IFI27, IFIT1, IFI44L, SIGLEC1, ISG15, | 0.888 | 0.879 | 0.897 | 0.935 | 0.884 | 0.986 | 0.951 | 0.882 | 1 |
| IL1R2, FAM20A, RETN, MMP8 | | | | | | | | | |
| RSAD2, IFI27, SIGLEC1, ISG15, IL1R2, | 0.888 | 0.878 | 0.898 | 0.934 | 0.881 | 0.987 | 0.951 | 0.889 | 1 |
| OLAH, FAM20A, RETN, MMP8 | | | | | | | | | |
| IFI27, SIGLEC1, ISG15, HERC6, IL1R2, | 0.888 | 0.878 | 0.898 | 0.933 | 0.879 | 0.987 | 0.953 | 0.891 | 1 |
| OLAH, FAM20A, RETN, MMP8 | | | | | | | | | |
| IFI27, IFI44L, SIGLEC1, ISG15, HERC6, | 0.888 | 0.878 | 0.897 | 0.935 | 0.884 | 0.987 | 0.950 | 0.880 | 1 |
| IL1R2, FAM20A, RETN, MMP8 | | | | | | | | | |
| RSAD2, IFI27, OAS1, IFIT1, SIGLEC1, | 0.888 | 0.879 | 0.897 | 0.932 | 0.879 | 0.985 | 0.939 | 0.874 | 1 |
| HERC6, OLAH, FAM20A, RETN | | | | | | | | | |
| IFI27, IFIT1, IFI44L, SIGLEC1, HERC6, | 0.888 | 0.879 | 0.897 | 0.932 | 0.878 | 0.986 | 0.945 | 0.881 | 1 |
| IL1R2, OLAH, FAM20A, RETN | | | | | | | | | |
| RSAD2, IFI27, IFIT1, SIGLEC1, ISG15, | 0.888 | 0.879 | 0.896 | 0.934 | 0.882 | 0.986 | 0.940 | 0.875 | 1 |
| HERC6, IL1R2, OLAH, FAM20A | | | | | | | | | |
| IFI27, IFIT1, SIGLEC1, ISG15, HERC6, | 0.888 | 0.878 | 0.897 | 0.933 | 0.879 | 0.986 | 0.940 | 0.875 | 1 |
| IL1R2, OLAH, FAM20A, RETN | | | | | | | | | |
| RSAD2, IFI27, OAS1, IFI44L, HERC6, | 0.888 | 0.878 | 0.897 | 0.933 | 0.882 | 0.984 | 0.929 | 0.856 | 1 |
| IL1R2, OLAH, FAM20A, MMP8 | | | | | | | | | |
| IFI27, OAS1, IFI44L, ISG15, HERC6, | 0.888 | 0.878 | 0.898 | 0.931 | 0.879 | 0.983 | 0.927 | 0.851 | 1 |
| IL1R2, OLAH, FAM20A, MMP8 | | | | | | | | | |
| RSAD2, IFI27, OAS1, SIGLEC1, ISG15, | 0.887 | 0.878 | 0.897 | 0.931 | 0.878 | 0.985 | 0.940 | 0.874 | 1 |
| HERC6, IL1R2, OLAH, FAM20A | | | | | | | | | |
| RSAD2, IFI27, OAS1, IFIT1, IFI44L, | 0.887 | 0.879 | 0.896 | 0.931 | 0.880 | 0.982 | 0.957 | 0.896 | 1 |
| SIGLEC1, IL1R2, FAM20A, MMP8 | | | | | | | | | |
| IFI27, OAS1, IFIT1, IFI44L, HERC6, | 0.887 | 0.878 | 0.897 | 0.932 | 0.880 | 0.983 | 0.929 | 0.855 | 1 |
| IL1R2, OLAH, FAM20A, MMP8 | | | | | | | | | |
| RSAD2, IFI27, OAS1, IFI44L, SIGLEC1, | 0.887 | 0.878 | 0.896 | 0.933 | 0.881 | 0.984 | 0.943 | 0.877 | 1 |
| HERC6, IL1R2, OLAH, FAM20A | | | | | | | | | |
| IFI27, OAS1, IFIT1, SIGLEC1, HERC6, | 0.887 | 0.877 | 0.897 | 0.939 | 0.892 | 0.986 | 0.941 | 0.876 | 1 |
| SLC1A2, OLAH, FAM20A, MMP8 | | | | | | | | | |
| IFI27, IFI44L, SIGLEC1, HERC6, IL1R2, | 0.887 | 0.878 | 0.897 | 0.935 | 0.884 | 0.987 | 0.951 | 0.886 | 1 |
| OLAH, FAM20A, RETN, MMP8 | | | | | | | | | |
| RSAD2, IFI27, OAS1, IFIT1, SIGLEC1, | 0.887 | 0.878 | 0.897 | 0.933 | 0.879 | 0.987 | 0.942 | 0.873 | 1 |
| HERC6, FAM20A, RETN, MMP8 | | | | | | | | | |
| RSAD2, IFI27, IFIT1, IFI44L, SIGLEC1, | 0.887 | 0.878 | 0.897 | 0.932 | 0.878 | 0.986 | 0.941 | 0.878 | 1 |
| ISG15, HERC6, OLAH, FAM20A | | | | | | | | | |
| IFI27, OAS1, IFIT1, SIGLEC1, ISG15, | 0.887 | 0.878 | 0.896 | 0.934 | 0.883 | 0.986 | 0.954 | 0.891 | 1 |
| IL1R2, FAM20A, RETN, MMP8 | | | | | | | | | |
| IFI27, OAS1, IFI44L, SIGLEC1, ISG15, | 0.887 | 0.878 | 0.896 | 0.931 | 0.879 | 0.984 | 0.940 | 0.873 | 1 |
| HERC6, IL1R2, OLAH, FAM20A | | | | | | | | | |
| RSAD2, IFI27, OAS1, IFIT1, HERC6, | 0.887 | 0.877 | 0.897 | 0.932 | 0.881 | 0.983 | 0.933 | 0.862 | 1 |
| IL1R2, OLAH, FAM20A, MMP8 | | | | | | | | | |
| IFI27, OAS1, SIGLEC1, ISG15, HERC6, | 0.887 | 0.877 | 0.897 | 0.937 | 0.888 | 0.985 | 0.939 | 0.873 | 1 |
| SLC1A2, OLAH, FAM20A, MMP8 | | | | | | | | | |
| IFI27, IFIT1, IFI44L, SIGLEC1, SLC1A2, | 0.887 | 0.878 | 0.896 | 0.939 | 0.894 | 0.985 | 0.954 | 0.893 | 1 |
| IL1R2, OLAH, FAM20A, MMP8 | | | | | | | | | |
| RSAD2, IFI27, OAS1, SIGLEC1, ISG15, | 0.887 | 0.877 | 0.896 | 0.935 | 0.885 | 0.985 | 0.950 | 0.882 | 1 |
| HERC6, IL1R2, FAM20A, MMP8 | | | | | | | | | |
| IFI27, IFIT1, IFI44L, SIGLEC1, ISG15, | 0.887 | 0.877 | 0.896 | 0.939 | 0.891 | 0.987 | 0.947 | 0.883 | 1 |
| SLC1A2, OLAH, FAM20A, MMP8 | | | | | | | | | |
| IFI27, OAS1, IFIT1, IFI44L, SIGLEC1, | 0.887 | 0.877 | 0.897 | 0.933 | 0.879 | 0.987 | 0.942 | 0.873 | 1 |
| HERC6, FAM20A, RETN, MMP8 | | | | | | | | | |
| IFI27, IFI44L, SIGLEC1, ISG15, HERC6, | 0.887 | 0.877 | 0.897 | 0.931 | 0.875 | 0.987 | 0.947 | 0.881 | 1 |
| OLAH, FAM20A, RETN, MMP8 | | | | | | | | | |
| RSAD2, IFI27, IFIT1, SIGLEC1, IL1R2, | 0.887 | 0.877 | 0.897 | 0.934 | 0.880 | 0.987 | 0.954 | 0.895 | 1 |
| OLAH, FAM20A, RETN, MMP8 | | | | | | | | | |
| IFI27, IFIT1, IFI44L, SIGLEC1, ISG15, | 0.887 | 0.877 | 0.896 | 0.927 | 0.871 | 0.984 | 0.947 | 0.875 | 1 |
| HERC6, FAM20A, RETN, MMP8 | | | | | | | | | |
| RSAD2, IFI27, IFI44L, SIGLEC1, | 0.887 | 0.877 | 0.897 | 0.937 | 0.887 | 0.987 | 0.949 | 0.879 | 1 |
| HERC6, IL1R2, FAM20A, RETN, MMP8 | | | | | | | | | |
| RSAD2, IFI27, IFIT1, IFI44L, SIGLEC1, | 0.887 | 0.878 | 0.896 | 0.931 | 0.877 | 0.986 | 0.939 | 0.875 | 1 |
| HERC6, OLAH, FAM20A, RETN | | | | | | | | | |
| IFI27, SIGLEC1, ISG15, HERC6, | 0.887 | 0.877 | 0.896 | 0.940 | 0.894 | 0.985 | 0.950 | 0.885 | 1 |
| SLC1A2, IL1R2, OLAH, FAM20A, MMP8 | | | | | | | | | |
| IFI27, OAS1, IFIT1, IFI44L, SIGLEC1, | 0.887 | 0.878 | 0.895 | 0.933 | 0.883 | 0.983 | 0.947 | 0.881 | 1 |
| ISG15, HERC6, IL1R2, FAM20A | | | | | | | | | |
| IFI27, OAS1, IFIT1, SIGLEC1, ISG15, | 0.887 | 0.877 | 0.897 | 0.931 | 0.874 | 0.988 | 0.954 | 0.900 | 1 |
| OLAH, FAM20A, RETN, MMP8 | | | | | | | | | |
| IFI27, OAS1, IFI44L, SIGLEC1, HERC6, | 0.887 | 0.876 | 0.897 | 0.935 | 0.882 | 0.988 | 0.948 | 0.884 | 1 |
| OLAH, FAM20A, RETN, MMP8 | | | | | | | | | |
| RSAD2, IFI27, IFI44L, SIGLEC1, ISG15, | 0.887 | 0.878 | 0.896 | 0.934 | 0.883 | 0.984 | 0.951 | 0.883 | 1 |
| HERC6, IL1R2, FAM20A, MMP8 | | | | | | | | | |
| RSAD2, IFI27, IFIT1, IFI44L, HERC6, | 0.887 | 0.877 | 0.896 | 0.933 | 0.881 | 0.984 | 0.930 | 0.857 | 1 |
| IL1R2, OLAH, FAM20A, MMP8 | | | | | | | | | |
| IFI27, IFIT1, IFI44L, SIGLEC1, ISG15, | 0.887 | 0.877 | 0.896 | 0.930 | 0.874 | 0.986 | 0.955 | 0.900 | 1 |
| OLAH, FAM20A, RETN, MMP8 | | | | | | | | | |
| RSAD2, IFI27, OAS1, IFIT1, IFI44L, | 0.887 | 0.878 | 0.895 | 0.935 | 0.886 | 0.983 | 0.946 | 0.879 | 1 |
| SIGLEC1, HERC6, IL1R2, FAM20A | | | | | | | | | |
| IFI27, OAS1, IFIT1, SIGLEC1, ISG15, | 0.886 | 0.876 | 0.896 | 0.939 | 0.891 | 0.987 | 0.943 | 0.879 | 1 |
| SLC1A2, OLAH, FAM20A, MMP8 | | | | | | | | | |
| RSAD2, IFI27, IFI44L, SIGLEC1, | 0.886 | 0.877 | 0.896 | 0.934 | 0.881 | 0.988 | 0.950 | 0.890 | 1 |
| HERC6, OLAH, FAM20A, RETN, MMP8 | | | | | | | | | |
| RSAD2, IFI27, IFIT1, SIGLEC1, HERC6, | 0.886 | 0.877 | 0.895 | 0.932 | 0.879 | 0.986 | 0.939 | 0.874 | 1 |
| IL1R2, OLAH, FAM20A, RETN | | | | | | | | | |
| RSAD2, IFI27, IFIT1, SIGLEC1, ISG15, | 0.886 | 0.877 | 0.895 | 0.931 | 0.878 | 0.985 | 0.941 | 0.869 | 1 |
| HERC6, FAM20A, RETN, MMP8 | | | | | | | | | |
| RSAD2, IFI27, SIGLEC1, ISG15, | 0.886 | 0.877 | 0.896 | 0.942 | 0.898 | 0.985 | 0.949 | 0.884 | 1 |
| SLC1A2, IL1R2, OLAH, FAM20A, MMP8 | | | | | | | | | |
| IFI27, OAS1, IFIT1, IFI44L, SIGLEC1, | 0.886 | 0.877 | 0.896 | 0.937 | 0.889 | 0.984 | 0.935 | 0.867 | 1 |
| HERC6, SLC1A2, OLAH, FAM20A | | | | | | | | | |
| RSAD2, IFI27, IFIT1, SIGLEC1, | 0.886 | 0.877 | 0.895 | 0.939 | 0.893 | 0.985 | 0.951 | 0.889 | 1 |
| SLC1A2, IL1R2, OLAH, FAM20A, MMP8 | | | | | | | | | |
| IFI27, IFIT1, IFI44L, SIGLEC1, ISG15, | 0.886 | 0.877 | 0.895 | 0.932 | 0.879 | 0.984 | 0.946 | 0.878 | 1 |
| HERC6, IL1R2, FAM20A, RETN | | | | | | | | | |
| RSAD2, IFI27, IFI44L, SIGLEC1, ISG15, | 0.886 | 0.876 | 0.896 | 0.934 | 0.881 | 0.987 | 0.946 | 0.884 | 1 |
| HERC6, OLAH, FAM20A, MMP8 | | | | | | | | | |
| RSAD2, IFI27, IFIT1, SIGLEC1, ISG15, | 0.886 | 0.877 | 0.895 | 0.931 | 0.877 | 0.985 | 0.938 | 0.874 | 1 |
| HERC6, OLAH, FAM20A, RETN | | | | | | | | | |
| RSAD2, IFI27, IFI44L, ISG15, HERC6, | 0.886 | 0.876 | 0.896 | 0.931 | 0.878 | 0.983 | 0.928 | 0.854 | 1 |
| IL1R2, OLAH, FAM20A, MMP8 | | | | | | | | | |
| RSAD2, IFI27, IFIT1, SIGLEC1, ISG15, | 0.886 | 0.877 | 0.896 | 0.939 | 0.890 | 0.987 | 0.942 | 0.876 | 1 |
| SLC1A2, OLAH, FAM20A, MMP8 | | | | | | | | | |
| IFI27, OAS1, IFIT1, SIGLEC1, SLC1A2, | 0.886 | 0.877 | 0.895 | 0.939 | 0.893 | 0.986 | 0.947 | 0.881 | 1 |
| IL1R2, OLAH, FAM20A, MMP8 | | | | | | | | | |
| IFI27, OAS1, SIGLEC1, ISG15, SLC1A2, | 0.886 | 0.877 | 0.896 | 0.940 | 0.895 | 0.985 | 0.947 | 0.879 | 1 |
| IL1R2, OLAH, FAM20A, MMP8 | | | | | | | | | |
| RSAD2, IFI27, SIGLEC1, HERC6, | 0.886 | 0.877 | 0.896 | 0.938 | 0.892 | 0.984 | 0.948 | 0.884 | 1 |
| SLC1A2, IL1R2, OLAH, FAM20A, MMP8 | | | | | | | | | |
| IFI27, IFI44L, SIGLEC1, ISG15, HERC6, | 0.886 | 0.877 | 0.895 | 0.930 | 0.877 | 0.983 | 0.942 | 0.877 | 1 |
| IL1R2, OLAH, FAM20A, RETN | | | | | | | | | |
| RSAD2, IFI27, OAS1, ISG15, HERC6, | 0.886 | 0.876 | 0.896 | 0.931 | 0.879 | 0.982 | 0.924 | 0.847 | 1 |
| IL1R2, OLAH, FAM20A, MMP8 | | | | | | | | | |
| IFI27, OAS1, IFIT1, IFI44L, SIGLEC1, | 0.886 | 0.876 | 0.896 | 0.928 | 0.870 | 0.986 | 0.959 | 0.907 | 1 |
| OLAH, FAM20A, RETN, MMP8 | | | | | | | | | |
| IFI27, IFIT1, SIGLEC1, ISG15, HERC6, | 0.886 | 0.876 | 0.896 | 0.939 | 0.893 | 0.986 | 0.950 | 0.887 | 1 |
| SLC1A2, OLAH, FAM20A, MMP8 | | | | | | | | | |
| IFI27, IFIT1, IFI44L, SIGLEC1, ISG15, | 0.886 | 0.878 | 0.895 | 0.929 | 0.873 | 0.985 | 0.947 | 0.886 | 1 |
| IL1R2, OLAH, FAM20A, RETN | | | | | | | | | |
| RSAD2, IFI27, IFIT1, IFI44L, SIGLEC1, | 0.886 | 0.876 | 0.896 | 0.930 | 0.873 | 0.986 | 0.949 | 0.891 | 1 |
| ISG15, OLAH, FAM20A, MMP8 | | | | | | | | | |
| IFI27, OAS1, IFIT1, ISG15, HERC6, | 0.886 | 0.876 | 0.896 | 0.930 | 0.878 | 0.982 | 0.933 | 0.862 | 1 |
| IL1R2, OLAH, FAM20A, MMP8 | | | | | | | | | |
| IFI27, OAS1, IFIT1, IFI44L, SIGLEC1, | 0.886 | 0.876 | 0.896 | 0.938 | 0.888 | 0.988 | 0.947 | 0.887 | 1 |
| SLC1A2, OLAH, FAM20A, MMP8 | | | | | | | | | |
| RSAD2, IFI27, OAS1, IFIT1, IFI44L, | 0.886 | 0.876 | 0.896 | 0.931 | 0.877 | 0.984 | 0.934 | 0.866 | 1 |
| HERC6, OLAH, FAM20A, MMP8 | | | | | | | | | |
| IFI27, OAS1, IFIT1, IFI44L, SIGLEC1, | 0.886 | 0.877 | 0.895 | 0.926 | 0.870 | 0.983 | 0.948 | 0.885 | 1 |
| IL1R2, OLAH, FAM20A, RETN | | | | | | | | | |
| RSAD2, IFI27, OAS1, IFIT1, SIGLEC1, | 0.886 | 0.877 | 0.895 | 0.930 | 0.877 | 0.984 | 0.934 | 0.861 | 1 |
| ISG15, HERC6, FAM20A, RETN | | | | | | | | | |
| IFI27, OAS1, IFIT1, SIGLEC1, ISG15, | 0.886 | 0.877 | 0.895 | 0.934 | 0.884 | 0.985 | 0.943 | 0.876 | 1 |
| HERC6, IL1R2, FAM20A, RETN | | | | | | | | | |
| IFI27, OAS1, ISG15, HERC6, IL1R2, | 0.886 | 0.876 | 0.895 | 0.934 | 0.881 | 0.986 | 0.932 | 0.858 | 1 |
| OLAH, FAM20A, RETN, MMP8 | | | | | | | | | |
| IFI27, OAS1, SIGLEC1, HERC6, | 0.886 | 0.876 | 0.896 | 0.938 | 0.892 | 0.985 | 0.940 | 0.871 | 1 |
| SLC1A2, IL1R2, OLAH, FAM20A, MMP8 | | | | | | | | | |
| IFI27, OAS1, IFIT1, HERC6, IL1R2, | 0.886 | 0.876 | 0.895 | 0.934 | 0.882 | 0.986 | 0.929 | 0.855 | 1 |
| OLAH, FAM20A, RETN, MMP8 | | | | | | | | | |
| IFI27, OAS1, IFIT1, IFI44L, SIGLEC1, | 0.886 | 0.876 | 0.895 | 0.933 | 0.882 | 0.983 | 0.952 | 0.885 | 1 |
| ISG15, IL1R2, FAM20A, MMP8 | | | | | | | | | |
| RSAD2, IFI27, IFI44L, SIGLEC1, ISG15, | 0.886 | 0.877 | 0.895 | 0.931 | 0.878 | 0.984 | 0.939 | 0.872 | 1 |
| HERC6, IL1R2, OLAH, FAM20A | | | | | | | | | |
| RSAD2, IFI27, OAS1, IFI44L, SIGLEC1, | 0.886 | 0.876 | 0.895 | 0.930 | 0.876 | 0.985 | 0.940 | 0.874 | 1 |
| ISG15, HERC6, OLAH, FAM20A | | | | | | | | | |
| RSAD2, IFI27, OAS1, IFIT1, SIGLEC1, | 0.886 | 0.877 | 0.894 | 0.936 | 0.886 | 0.985 | 0.942 | 0.874 | 1 |
| HERC6, IL1R2, FAM20A, RETN | | | | | | | | | |
| IFI27, OAS1, SIGLEC1, ISG15, HERC6, | 0.886 | 0.876 | 0.895 | 0.931 | 0.878 | 0.984 | 0.939 | 0.871 | 1 |
| IL1R2, OLAH, FAM20A, RETN | | | | | | | | | |
| RSAD2, IFI27, IFIT1, SIGLEC1, HERC6, | 0.886 | 0.876 | 0.896 | 0.938 | 0.891 | 0.985 | 0.948 | 0.885 | 1 |
| SLC1A2, OLAH, FAM20A, MMP8 | | | | | | | | | |
| RSAD2, IFI27, OAS1, HERC6, IL1R2, | 0.886 | 0.876 | 0.895 | 0.934 | 0.883 | 0.986 | 0.932 | 0.858 | 1 |
| OLAH, FAM20A, RETN, MMP8 | | | | | | | | | |
| RSAD2, IFI27, IFIT1, SIGLEC1, ISG15, | 0.886 | 0.877 | 0.895 | 0.936 | 0.884 | 0.987 | 0.952 | 0.886 | 1 |
| IL1R2, FAM20A, RETN, MMP8 | | | | | | | | | |
| RSAD2, IFI27, OAS1, IFIT1, SIGLEC1, | 0.885 | 0.876 | 0.895 | 0.934 | 0.882 | 0.986 | 0.955 | 0.892 | 1 |
| IL1R2, FAM20A, RETN, MMP8 | | | | | | | | | |
| RSAD2, IFI27, OAS1, IFIT1, SIGLEC1, | 0.885 | 0.876 | 0.895 | 0.929 | 0.872 | 0.986 | 0.954 | 0.900 | 1 |
| OLAH, FAM20A, RETN, MMP8 | | | | | | | | | |
| IFI27, OAS1, IFIT1, SIGLEC1, ISG15, | 0.885 | 0.876 | 0.894 | 0.929 | 0.872 | 0.985 | 0.945 | 0.883 | 1 |
| IL1R2, OLAH, FAM20A, RETN | | | | | | | | | |
| IFI27, OAS1, IFI44L, SIGLEC1, HERC6, | 0.885 | 0.876 | 0.895 | 0.932 | 0.880 | 0.984 | 0.943 | 0.878 | 1 |
| IL1R2, OLAH, FAM20A, RETN | | | | | | | | | |
| RSAD2, IFI27, IFIT1, IFI44L, SIGLEC1, | 0.885 | 0.876 | 0.894 | 0.929 | 0.876 | 0.983 | 0.936 | 0.862 | 1 |
| ISG15, HERC6, FAM20A, RETN | | | | | | | | | |
| IFI27, OAS1, IFIT1, IFI44L, SIGLEC1, | 0.885 | 0.877 | 0.894 | 0.928 | 0.873 | 0.984 | 0.946 | 0.883 | 1 |
| ISG15, IL1R2, OLAH, FAM20A | | | | | | | | | |
| IFI27, IFIT1, SIGLEC1, HERC6, | 0.885 | 0.876 | 0.895 | 0.939 | 0.894 | 0.985 | 0.951 | 0.887 | 1 |
| SLC1A2, IL1R2, OLAH, FAM20A, MMP8 | | | | | | | | | |
| RSAD2, IFI27, OAS1, SIGLEC1, HERC6, | 0.885 | 0.875 | 0.895 | 0.935 | 0.886 | 0.985 | 0.940 | 0.875 | 1 |
| SLC1A2, OLAH, FAM20A, MMP8 | | | | | | | | | |
| IFI27, OAS1, SIGLEC1, HERC6, | 0.885 | 0.875 | 0.896 | 0.937 | 0.888 | 0.986 | 0.943 | 0.879 | 1 |
| SLC1A2, OLAH, FAM20A, RETN, MMP8 | | | | | | | | | |
| RSAD2, IFI27, OAS1, IFIT1, SIGLEC1, | 0.885 | 0.877 | 0.894 | 0.935 | 0.886 | 0.984 | 0.948 | 0.882 | 1 |
| ISG15, HERC6, IL1R2, FAM20A | | | | | | | | | |
| RSAD2, IFI27, SIGLEC1, ISG15, HERC6, | 0.885 | 0.876 | 0.895 | 0.931 | 0.877 | 0.985 | 0.941 | 0.877 | 1 |
| IL1R2, OLAH, FAM20A, RETN | | | | | | | | | |
| IFI27, IFIT1, IFI44L, ISG15, HERC6, | 0.885 | 0.876 | 0.895 | 0.930 | 0.877 | 0.982 | 0.929 | 0.857 | 1 |
| IL1R2, OLAH, FAM20A, MMP8 | | | | | | | | | |
| IFI27, IFIT1, IFI44L, SIGLEC1, HERC6, | 0.885 | 0.876 | 0.895 | 0.935 | 0.886 | 0.985 | 0.954 | 0.894 | 1 |
| SLC1A2, OLAH, FAM20A, MMP8 | | | | | | | | | |
| IFI27, OAS1, IFI44L, SIGLEC1, IL1R2, | 0.885 | 0.876 | 0.895 | 0.934 | 0.881 | 0.988 | 0.953 | 0.896 | 1 |
| OLAH, FAM20A, RETN, MMP8 | | | | | | | | | |
| RSAD2, IFI27, OAS1, SIGLEC1, ISG15, | 0.885 | 0.876 | 0.895 | 0.930 | 0.875 | 0.984 | 0.940 | 0.874 | 1 |
| HERC6, OLAH, FAM20A, RETN | | | | | | | | | |
| RSAD2, IFI27, IFIT1, HERC6, IL1R2, | 0.885 | 0.876 | 0.895 | 0.933 | 0.880 | 0.985 | 0.932 | 0.860 | 1 |
| OLAH, FAM20A, RETN, MMP8 | | | | | | | | | |
| RSAD2, IFI27, OAS1, SIGLEC1, | 0.885 | 0.876 | 0.895 | 0.938 | 0.891 | 0.985 | 0.947 | 0.883 | 1 |
| SLC1A2, IL1R2, OLAH, FAM20A, MMP8 | | | | | | | | | |
| RSAD2, IFI27, OAS1, SIGLEC1, ISG15, | 0.885 | 0.875 | 0.896 | 0.931 | 0.874 | 0.988 | 0.950 | 0.893 | 1 |
| OLAH, FAM20A, RETN, MMP8 | | | | | | | | | |
| RSAD2, IFI27, OAS1, IFIT1, IFI44L, | 0.885 | 0.876 | 0.894 | 0.930 | 0.877 | 0.984 | 0.934 | 0.861 | 1 |
| SIGLEC1, HERC6, FAM20A, RETN | | | | | | | | | |
| RSAD2, IFI27, OAS1, IFIT1, SIGLEC1, | 0.885 | 0.876 | 0.894 | 0.933 | 0.882 | 0.983 | 0.954 | 0.893 | 1 |
| ISG15, IL1R2, FAM20A, MMP8 | | | | | | | | | |
| IFI27, OAS1, IFIT1, SIGLEC1, HERC6, | 0.885 | 0.875 | 0.895 | 0.937 | 0.893 | 0.980 | 0.951 | 0.884 | 1 |
| SLC1A2, IL1R2, FAM20A, MMP8 | | | | | | | | | |
| RSAD2, IFI27, OAS1, SIGLEC1, HERC6, | 0.885 | 0.875 | 0.895 | 0.931 | 0.878 | 0.984 | 0.941 | 0.875 | 1 |
| IL1R2, OLAH, FAM20A, RETN | | | | | | | | | |
| RSAD2, IFI27, SIGLEC1, ISG15, HERC6, | 0.885 | 0.875 | 0.895 | 0.938 | 0.891 | 0.985 | 0.942 | 0.876 | 1 |
| SLC1A2, OLAH, FAM20A, MMP8 | | | | | | | | | |
| IFI27, OAS1, IFI44L, SIGLEC1, ISG15, | 0.885 | 0.875 | 0.894 | 0.934 | 0.883 | 0.986 | 0.949 | 0.879 | 1 |
| IL1R2, FAM20A, RETN, MMP8 | | | | | | | | | |
| IFI27, OAS1, IFIT1, IFI44L, SIGLEC1, | 0.885 | 0.876 | 0.894 | 0.927 | 0.870 | 0.985 | 0.941 | 0.878 | 1 |
| ISG15, OLAH, FAM20A, RETN | | | | | | | | | |
| RSAD2, IFI27, OAS1, SIGLEC1, ISG15, | 0.885 | 0.875 | 0.895 | 0.937 | 0.887 | 0.986 | 0.940 | 0.874 | 1 |
| SLC1A2, OLAH, FAM20A, MMP8 | | | | | | | | | |
| IFI27, IFIT1, IFI44L, SIGLEC1, ISG15, | 0.885 | 0.875 | 0.894 | 0.938 | 0.82 | 0.984 | 0.937 | 0.870 | 1 |
| HERC6, SLC1A2, OLAH, FAM20A | | | | | | | | | |
| RSAD2, IFI27, OAS1, IFIT1, HERC6, | 0.885 | 0.875 | 0.895 | 0.931 | 0.877 | 0.986 | 0.930 | 0.858 | 1 |
| OLAH, FAM20A, RETN, MMP8 | | | | | | | | | |
| RSAD2, IFI27, OAS1, IFIT1, IFI44L, | 0.885 | 0.876 | 0.894 | 0.926 | 0.868 | 0.984 | 0.943 | 0.883 | 1 |
| SIGLEC1, ISG15, OLAH, FAM20A | | | | | | | | | |
| RSAD2, IFI27, IFI44L, SIGLEC1, IL1R2, | 0.885 | 0.875 | 0.895 | 0.935 | 0.882 | 0.988 | 0.951 | 0.890 | 1 |
| OLAH, FAM20A, RETN, MMP8 | | | | | | | | | |
| IFI27, IFI44L, SIGLEC1, ISG15, HERC6, | 0.885 | 0.875 | 0.894 | 0.937 | 0.889 | 0.985 | 0.947 | 0.882 | 1 |
| SLC1A2, OLAH, FAM20A, MMP8 | | | | | | | | | |
| RSAD2, IFI27, IFIT1, IFI44L, SIGLEC1, | 0.885 | 0.875 | 0.894 | 0.931 | 0.877 | 0.985 | 0.942 | 0.870 | 1 |
| HERC6, FAM20A, RETN, MMP8 | | | | | | | | | |
| IFI27, SIGLEC1, ISG15, SLC1A2, IL1R2, | 0.885 | 0.875 | 0.894 | 0.942 | 0.898 | 0.986 | 0.946 | 0.875 | 1 |
| OLAH, FAM20A, RETN, MMP8 | | | | | | | | | |
| RSAD2, IFI27, IFIT1, IFI44L, SIGLEC1, | 0.885 | 0.876 | 0.894 | 0.932 | 0.882 | 0.983 | 0.952 | 0.885 | 1 |
| ISG15, IL1R2, FAM20A, MMP8 | | | | | | | | | |
| RSAD2, IFI27, IFIT1, SIGLEC1, ISG15, | 0.885 | 0.875 | 0.895 | 0.931 | 0.875 | 0.988 | 0.954 | 0.900 | 1 |
| OLAH, FAM20A, RETN, MMP8 | | | | | | | | | |
| IFI27, OAS1, IFI44L, SIGLEC1, ISG15, | 0.885 | 0.875 | 0.895 | 0.929 | 0.872 | 0.986 | 0.950 | 0.891 | 1 |
| OLAH, FAM20A, RETN, MMP8 | | | | | | | | | |
| RSAD2, IFI27, OAS1, IFI44L, SIGLEC1, | 0.885 | 0.874 | 0.895 | 0.930 | 0.873 | 0.986 | 0.949 | 0.890 | 1 |
| ISG15, OLAH, FAM20A, MMP8 | | | | | | | | | |
| IFI27, OAS1, IFI44L, SIGLEC1, ISG15, | 0.885 | 0.875 | 0.894 | 0.930 | 0.877 | 0.984 | 0.943 | 0.878 | 1 |
| HERC6, OLAH, FAM20A, RETN | | | | | | | | | |
| RSAD2, IFI27, OAS1, IFIT1, IFI44L, | 0.885 | 0.875 | 0.894 | 0.931 | 0.878 | 0.983 | 0.928 | 0.854 | 1 |
| HERC6, IL1R2, OLAH, FAM20A | | | | | | | | | |
| IFI27, IFIT1, IFI44L, HERC6, IL1R2, | 0.885 | 0.875 | 0.894 | 0.931 | 0.878 | 0.985 | 0.934 | 0.861 | 1 |
| OLAH, FAM20A, RETN, MMP8 | | | | | | | | | |
| RSAD2, IFI27, IFIT1, IFI44L, SIGLEC1, | 0.885 | 0.875 | 0.894 | 0.935 | 0.885 | 0.985 | 0.945 | 0.881 | 1 |
| SLC1A2, OLAH, FAM20A, MMP8 | | | | | | | | | |
| RSAD2, IFI27, IFIT1, ISG15, HERC6, | 0.885 | 0.875 | 0.894 | 0.930 | 0.878 | 0.982 | 0.932 | 0.862 | 1 |
| IL1R2, OLAH, FAM20A, MMP8 | | | | | | | | | |
| RSAD2, IFI27, OAS1, IFIT1, SIGLEC1, | 0.885 | 0.875 | 0.894 | 0.937 | 0.887 | 0.986 | 0.946 | 0.883 | 1 |
| SLC1A2, OLAH, FAM20A, MMP8 | | | | | | | | | |
| RSAD2, IFI27, OAS1, SIGLEC1, ISG15, | 0.885 | 0.875 | 0.894 | 0.935 | 0.883 | 0.987 | 0.950 | 0.883 | 1 |
| IL1R2, FAM20A, RETN, MMP8 | | | | | | | | | |
| RSAD2, IFI27, OAS1, IFIT1, IFI44L, | 0.885 | 0.876 | 0.893 | 0.926 | 0.870 | 0.983 | 0.948 | 0.885 | 1 |
| SIGLEC1, IL1R2, OLAH, FAM20A | | | | | | | | | |
| RSAD2, IFI27, ISG15, HERC6, IL1R2, | 0.885 | 0.875 | 0.894 | 0.933 | 0.881 | 0.986 | 0.930 | 0.857 | 1 |
| OLAH, FAM20A, RETN, MMP8 | | | | | | | | | |
| IFI27, OAS1, IFIT1, SIGLEC1, ISG15, | 0.884 | 0.874 | 0.895 | 0.939 | 0.894 | 0.985 | 0.930 | 0.860 | 1 |
| HERC6, SLC1A2, OLAH, FAM20A | | | | | | | | | |
| RSAD2, IFI27, IFIT1, IFI44L, SIGLEC1, | 0.884 | 0.876 | 0.893 | 0.933 | 0.882 | 0.983 | 0.943 | 0.875 | 1 |
| ISG15, HERC6, IL1R2, FAM20A | | | | | | | | | |
| IFI27, OAS1, IFI44L, HERC6, IL1R2, | 0.884 | 0.874 | 0.895 | 0.933 | 0.881 | 0.986 | 0.930 | 0.857 | 1 |
| OLAH, FAM20A, RETN, MMP8 | | | | | | | | | |
| RSAD2, IFI27, IFI44L, HERC6, IL1R2, | 0.884 | 0.874 | 0.895 | 0.933 | 0.881 | 0.985 | 0.934 | 0.863 | 1 |
| OLAH, FAM20A, RETN, MMP8 | | | | | | | | | |
| RSAD2, IFI27, OAS1, ISG15, HERC6, | 0.884 | 0.874 | 0.894 | 0.930 | 0.876 | 0.985 | 0.929 | 0.856 | 1 |
| OLAH, FAM20A, RETN, MMP8 | | | | | | | | | |
| IFI27, OAS1, IFIT1, IFI44L, HERC6, | 0.884 | 0.874 | 0.895 | 0.932 | 0.877 | 0.986 | 0.932 | 0.859 | 1 |
| OLAH, FAM20A, RETN, MMP8 | | | | | | | | | |
| RSAD2, IFI27, IFIT1, IFI44L, SIGLEC1, | 0.884 | 0.875 | 0.894 | 0.933 | 0.881 | 0.985 | 0.948 | 0.880 | 1 |
| HERC6, IL1R2, FAM20A, RETN | | | | | | | | | |
| RSAD2, IFI27, OAS1, IFIT1, SIGLEC1, | 0.884 | 0.876 | 0.893 | 0.929 | 0.873 | 0.984 | 0.945 | 0.881 | 1 |
| ISG15, IL1R2, OLAH, FAM20A | | | | | | | | | |
| RSAD2, IFI27, OAS1, IFI44L, SIGLEC1, | 0.884 | 0.875 | 0.894 | 0.933 | 0.883 | 0.983 | 0.950 | 0.882 | 1 |
| ISG15, IL1R2, FAM20A, MMP8 | | | | | | | | | |
| IFI27, OAS1, IFI44L, SIGLEC1, ISG15, | 0.884 | 0.874 | 0.894 | 0.937 | 0.888 | 0.986 | 0.940 | 0.873 | 1 |
| SLC1A2, OLAH, FAM20A, MMP8 | | | | | | | | | |
| IFI27, IFI44L, ISG15, HERC6, IL1R2, | 0.884 | 0.874 | 0.894 | 0.932 | 0.879 | 0.985 | 0.934 | 0.862 | 1 |
| OLAH, FAM20A, RETN, MMP8 | | | | | | | | | |
| RSAD2, IFI27, OAS1, SIGLEC1, ISG15, | 0.884 | 0.874 | 0.894 | 0.932 | 0.877 | 0.987 | 0.942 | 0.872 | 1 |
| HERC6, FAM20A, RETN, MMP8 | | | | | | | | | |
| IFI27, OAS1, IFI44L, SIGLEC1, ISG15, | 0.884 | 0.875 | 0.893 | 0.928 | 0.872 | 0.983 | 0.949 | 0.889 | 1 |
| IL1R2, OLAH, FAM20A, RETN | | | | | | | | | |
| RSAD2, IFI27, IFIT1, IFI44L, SIGLEC1, | 0.884 | 0.874 | 0.894 | 0.929 | 0.872 | 0.986 | 0.955 | 0.901 | 1 |
| OLAH, FAM20A, RETN, MMP8 | | | | | | | | | |
| IFI27, OAS1, SIGLEC1, HERC6, | 0.884 | 0.874 | 0.894 | 0.938 | 0.892 | 0.983 | 0.949 | 0.880 | 1 |
| SLC1A2, IL1R2, FAM20A, RETN, MMP8 | | | | | | | | | |
| RSAD2, IFI27, OAS1, IFIT1, SIGLEC1, | 0.884 | 0.874 | 0.894 | 0.937 | 0.890 | 0.984 | 0.939 | 0.874 | 1 |
| HERC6, SLC1A2, OLAH, FAM20A | | | | | | | | | |
| RSAD2, IFI27, OAS1, IFI44L, ISG15, | 0.884 | 0.873 | 0.895 | 0.930 | 0.876 | 0.984 | 0.933 | 0.865 | 1 |
| HERC6, OLAH, FAM20A, MMP8 | | | | | | | | | |
| RSAD2, IFI27, OAS1, IFI44L, ISG15, | 0.884 | 0.874 | 0.893 | 0.930 | 0.877 | 0.983 | 0.928 | 0.855 | 1 |
| HERC6, IL1R2, OLAH, FAM20A | | | | | | | | | |
| IFI27, OAS1, IFIT1, IFI44L, SIGLEC1, | 0.884 | 0.874 | 0.893 | 0.935 | 0.889 | 0.980 | 0.951 | 0.884 | 1 |
| SLC1A2, IL1R2, FAM20A, MMP8 | | | | | | | | | |
| RSAD2, IFI27, IFIT1, IFI44L, SIGLEC1, | 0.884 | 0.875 | 0.892 | 0.930 | 0.876 | 0.984 | 0.946 | 0.884 | 1 |
| ISG15, IL1R2, OLAH, FAM20A | | | | | | | | | |
| RSAD2, IFI27, OAS1, IFI44L, SIGLEC1, | 0.884 | 0.875 | 0.893 | 0.927 | 0.872 | 0.983 | 0.949 | 0.889 | 1 |
| ISG15, IL1R2, OLAH, FAM20A | | | | | | | | | |
| RSAD2, IFI27, OAS1, IFI44L, SIGLEC1, | 0.884 | 0.874 | 0.893 | 0.932 | 0.879 | 0.985 | 0.939 | 0.871 | 1 |
| HERC6, OLAH, FAM20A, RETN | | | | | | | | | |
| IFI27, IFIT1, SIGLEC1, ISG15, SLC1A2, | 0.884 | 0.874 | 0.894 | 0.937 | 0.889 | 0.986 | 0.948 | 0.883 | 1 |
| OLAH, FAM20A, RETN, MMP8 | | | | | | | | | |
| IFI27, IFIT1, ISG15, HERC6, IL1R2, | 0.884 | 0.874 | 0.893 | 0.932 | 0.879 | 0.985 | 0.934 | 0.862 | 1 |
| OLAH, FAM20A, RETN, MMP8 | | | | | | | | | |
| IFI27, OAS1, IFIT1, IFI44L, ISG15, | 0.884 | 0.874 | 0.893 | 0.931 | 0.879 | 0.983 | 0.928 | 0.855 | 1 |
| HERC6, OLAH, FAM20A, RETN | | | | | | | | | |
| IFI27, OAS1, IFIT1, SIGLEC1, HERC6, | 0.884 | 0.874 | 0.893 | 0.938 | 0.893 | 0.984 | 0.938 | 0.871 | 1 |
| SLC1A2, IL1R2, OLAH, FAM20A | | | | | | | | | |
| RSAD2, IFI27, OAS1, IFIT1, IFI44L, | 0.884 | 0.874 | 0.893 | 0.929 | 0.875 | 0.983 | 0.934 | 0.864 | 1 |
| IL1R2, OLAH, FAM20A, MMP8 | | | | | | | | | |
| IFI27, OAS1, IFIT1, ISG15, HERC6, | 0.884 | 0.873 | 0.894 | 0.933 | 0.878 | 0.987 | 0.935 | 0.865 | 1 |
| OLAH, FAM20A, RETN, MMP8 | | | | | | | | | |
| IFI27, OAS1, IFIT1, IFI44L, ISG15, | 0.883 | 0.873 | 0.894 | 0.928 | 0.873 | 0.983 | 0.932 | 0.862 | 1 |
| HERC6, OLAH, FAM20A, MMP8 | | | | | | | | | |
| IFI27, OAS1, IFIT1, IFI44L, ISG15, | 0.883 | 0.874 | 0.893 | 0.929 | 0.876 | 0.982 | 0.928 | 0.855 | 1 |
| HERC6, IL1R2, OLAH, FAM20A | | | | | | | | | |
| RSAD2, IFI27, OAS1, IFI44L, HERC6, | 0.883 | 0.873 | 0.894 | 0.932 | 0.877 | 0.986 | 0.929 | 0.856 | 1 |
| OLAH, FAM20A, RETN, MMP8 | | | | | | | | | |
| RSAD2, IFI27, IFIT1, IFI44L, SIGLEC1, | 0.883 | 0.873 | 0.893 | 0.935 | 0.884 | 0.987 | 0.951 | 0.885 | 1 |
| IL1R2, FAM20A, RETN, MMP8 | | | | | | | | | |
| RSAD2, IFI27, IFIT1, IFI44L, HERC6, | 0.883 | 0.873 | 0.894 | 0.931 | 0.876 | 0.985 | 0.929 | 0.857 | 1 |
| OLAH, FAM20A, RETN, MMP8 | | | | | | | | | |
| RSAD2, IFI27, OAS1, SIGLEC1, ISG15, | 0.883 | 0.874 | 0.893 | 0.926 | 0.869 | 0.983 | 0.946 | 0.882 | 1 |
| IL1R2, OLAH, FAM20A, RETN | | | | | | | | | |
| RSAD2, IFI27, IFI44L, SIGLEC1, ISG15, | 0.883 | 0.873 | 0.893 | 0.935 | 0.884 | 0.987 | 0.949 | 0.879 | 1 |
| IL1R2, FAM20A, RETN, MMP8 | | | | | | | | | |
| RSAD2, IFI27, IFIT1, SIGLEC1, HERC6, | 0.883 | 0.874 | 0.893 | 0.938 | 0.894 | 0.982 | 0.953 | 0.885 | 1 |
| SLC1A2, IL1R2, FAM20A, MMP8 | | | | | | | | | |
| IFI27, OAS1, IFI44L, SIGLEC1, HERC6, | 0.883 | 0.873 | 0.894 | 0.936 | 0.887 | 0.985 | 0.942 | 0.878 | 1 |
| SLC1A2, OLAH, FAM20A, MMP8 | | | | | | | | | |
| RSAD2, IFI27, SIGLEC1, ISG15, HERC6, | 0.883 | 0.874 | 0.893 | 0.937 | 0.894 | 0.981 | 0.951 | 0.882 | 1 |
| SLC1A2, IL1R2, FAM20A, MMP8 | | | | | | | | | |
| IFI27, IFIT1, IFI44L, ISG15, HERC6, | 0.883 | 0.873 | 0.893 | 0.927 | 0.870 | 0.984 | 0.936 | 0.867 | 1 |
| OLAH, FAM20A, RETN, MMP8 | | | | | | | | | |
| RSAD2, IFI27, IFI44L, SIGLEC1, | 0.883 | 0.873 | 0.893 | 0.932 | 0.879 | 0.985 | 0.945 | 0.880 | 1 |
| HERC6, IL1R2, OLAH, FAM20A, RETN | | | | | | | | | |
| RSAD2, IFI27, OAS1, IFI44L, SIGLEC1, | 0.883 | 0.873 | 0.893 | 0.933 | 0.881 | 0.985 | 0.952 | 0.888 | 1 |
| IL1R2, FAM20A, RETN, MMP8 | | | | | | | | | |
| IFI27, OAS1, IFI44L, ISG15, HERC6, | 0.883 | 0.873 | 0.894 | 0.932 | 0.878 | 0.986 | 0.932 | 0.859 | 1 |
| OLAH, FAM20A, RETN, MMP8 | | | | | | | | | |
| RSAD2, IFI27, IFIT1, ISG15, HERC6, | 0.883 | 0.873 | 0.893 | 0.929 | 0.874 | 0.984 | 0.933 | 0.860 | 1 |
| OLAH, FAM20A, RETN, MMP8 | | | | | | | | | |
| RSAD2, IFI27, OAS1, IFIT1, ISG15, | 0.883 | 0.873 | 0.893 | 0.929 | 0.875 | 0.983 | 0.934 | 0.866 | 1 |
| HERC6, OLAH, FAM20A, MMP8 | | | | | | | | | |
| IFI27, IFI44L, SIGLEC1, HERC6, | 0.883 | 0.874 | 0.893 | 0.933 | 0.884 | 0.982 | 0.951 | 0.885 | 1 |
| SLC1A2, IL1R2, OLAH, FAM20A, MMP8 | | | | | | | | | |
| IFI27, OAS1, IFI44L, SIGLEC1, ISG15, | 0.883 | 0.874 | 0.892 | 0.935 | 0.885 | 0.984 | 0.943 | 0.876 | 1 |
| HERC6, IL1R2, FAM20A, RETN | | | | | | | | | |
| IFI27, OAS1, IFI44L, SIGLEC1, HERC6, | 0.883 | 0.873 | 0.893 | 0.936 | 0.892 | 0.981 | 0.948 | 0.879 | 1 |
| SLC1A2, IL1R2, FAM20A, MMP8 | | | | | | | | | |
| IFI27, IFIT1, IFI44L, SIGLEC1, HERC6, | 0.883 | 0.874 | 0.892 | 0.936 | 0.891 | 0.981 | 0.955 | 0.888 | 1 |
| SLC1A2, IL1R2, FAM20A, MMP8 | | | | | | | | | |
| RSAD2, IFI27, IFIT1, SIGLEC1, ISG15, | 0.883 | 0.874 | 0.892 | 0.933 | 0.881 | 0.986 | 0.949 | 0.882 | 1 |
| HERC6, IL1R2, FAM20A, RETN | | | | | | | | | |
| RSAD2, IFI27, OAS1, IFIT1, SIGLEC1, | 0.883 | 0.874 | 0.892 | 0.926 | 0.869 | 0.983 | 0.949 | 0.886 | 1 |
| IL1R2, OLAH, FAM20A, RETN | | | | | | | | | |
| RSAD2, IFI27, IFIT1, SIGLEC1, ISG15, | 0.883 | 0.874 | 0.892 | 0.929 | 0.872 | 0.985 | 0.945 | 0.881 | 1 |
| IL1R2, OLAH, FAM20A, RETN | | | | | | | | | |
| IFI27, IFIT1, IFI44L, SIGLEC1, ISG15, | 0.883 | 0.874 | 0.892 | 0.938 | 0.896 | 0.981 | 0.954 | 0.886 | 1 |
| SLC1A2, IL1R2, FAM20A, MMP8 | | | | | | | | | |
| RSAD2, IFI27, IFIT1, IFI44L, SIGLEC1, | 0.883 | 0.873 | 0.893 | 0.936 | 0.888 | 0.984 | 0.939 | 0.874 | 1 |
| HERC6, SLC1A2, OLAH, FAM20A | | | | | | | | | |
| IFI27, OAS1, IFI44L, SIGLEC1, SLC1A2, | 0.883 | 0.873 | 0.893 | 0.938 | 0.891 | 0.985 | 0.948 | 0.884 | 1 |
| IL1R2, OLAH, FAM20A, MMP8 | | | | | | | | | |
| IFI27, IFIT1, IFI44L, SIGLEC1, HERC6, | 0.883 | 0.874 | 0.892 | 0.935 | 0.887 | 0.983 | 0.941 | 0.876 | 1 |
| SLC1A2, IL1R2, OLAH, FAM20A | | | | | | | | | |
| RSAD2, IFI27, OAS1, IFIT1, ISG15, | 0.883 | 0.874 | 0.892 | 0.929 | 0.877 | 0.982 | 0.926 | 0.851 | 1 |
| HERC6, IL1R2, OLAH, FAM20A | | | | | | | | | |
| IFI27, OAS1, IFIT1, SIGLEC1, SLC1A2, | 0.883 | 0.873 | 0.893 | 0.938 | 0.892 | 0.983 | 0.950 | 0.880 | 1 |
| IL1R2, FAM20A, RETN, MMP8 | | | | | | | | | |
| RSAD2, IFI27, IFIT1, IFI44L, ISG15, | 0.883 | 0.873 | 0.893 | 0.930 | 0.876 | 0.984 | 0.933 | 0.865 | 1 |
| HERC6, OLAH, FAM20A, MMP8 | | | | | | | | | |
| IFI27, OAS1, IFIT1, IFI44L, SIGLEC1, | 0.883 | 0.873 | 0.892 | 0.933 | 0.882 | 0.985 | 0.937 | 0.870 | 1 |
| ISG15, SLC1A2, OLAH, FAM20A | | | | | | | | | |
| RSAD2, IFI27, IFI44L, SIGLEC1, | 0.883 | 0.873 | 0.893 | 0.937 | 0.890 | 0.984 | 0.949 | 0.886 | 1 |
| SLC1A2, IL1R2, OLAH, FAM20A, MMP8 | | | | | | | | | |
| RSAD2, IFI27, OAS1, IFI44L, SIGLEC1, | 0.883 | 0.874 | 0.892 | 0.933 | 0.884 | 0.982 | 0.946 | 0.880 | 1 |
| ISG15, HERC6, IL1R2, FAM20A | | | | | | | | | |
| RSAD2, IFI27, SIGLEC1, ISG15, | 0.883 | 0.873 | 0.893 | 0.938 | 0.890 | 0.987 | 0.945 | 0.880 | 1 |
| SLC1A2, OLAH, FAM20A, RETN, MMP8 | | | | | | | | | |
| RSAD2, IFI27, OAS1, IFIT1, IFI44L, | 0.883 | 0.873 | 0.893 | 0.924 | 0.865 | 0.983 | 0.951 | 0.891 | 1 |
| SIGLEC1, OLAH, FAM20A, RETN | | | | | | | | | |
| IFI27, IFIT1, IFI44L, SIGLEC1, ISG15, | 0.883 | 0.874 | 0.892 | 0.937 | 0.889 | 0.984 | 0.937 | 0.869 | 1 |
| SLC1A2, IL1R2, OLAH, FAM20A | | | | | | | | | |
| RSAD2, IFI27, SIGLEC1, SLC1A2, | 0.883 | 0.873 | 0.893 | 0.938 | 0.890 | 0.985 | 0.949 | 0.885 | 1 |
| IL1R2, OLAH, FAM20A, RETN, MMP8 | | | | | | | | | |
| RSAD2, IFI27, OAS1, IFIT1, IFI44L, | 0.883 | 0.873 | 0.893 | 0.930 | 0.875 | 0.984 | 0.927 | 0.855 | 0.999 |
| ISG15, HERC6, OLAH, FAM20A | | | | | | | | | |
| IFI27, OAS1, IFIT1, IFI44L, ISG15, | 0.883 | 0.873 | 0.893 | 0.929 | 0.876 | 0.981 | 0.935 | 0.867 | 1 |
| IL1R2, OLAH, FAM20A, MMP8 | | | | | | | | | |
| RSAD2, IFI27, SIGLEC1, HERC6, | 0.883 | 0.873 | 0.893 | 0.937 | 0.889 | 0.985 | 0.950 | 0.880 | 1 |
| SLC1A2, IL1R2, FAM20A, RETN, MMP8 | | | | | | | | | |
| IFI27, IFIT1, SIGLEC1, HERC6, | 0.883 | 0.873 | 0.893 | 0.938 | 0.889 | 0.986 | 0.951 | 0.887 | 1 |
| SLC1A2, OLAH, FAM20A, RETN, MMP8 | | | | | | | | | |
| IFI27, IFIT1, SIGLEC1, HERC6, | 0.883 | 0.873 | 0.892 | 0.938 | 0.892 | 0.985 | 0.954 | 0.886 | 1 |
| SLC1A2, IL1R2, FAM20A, RETN, MMP8 | | | | | | | | | |
| RSAD2, IFI27, IFI44L, SIGLEC1, ISG15, | 0.883 | 0.873 | 0.892 | 0.930 | 0.876 | 0.984 | 0.940 | 0.874 | 1 |
| HERC6, OLAH, FAM20A, RETN | | | | | | | | | |
| IFI27, IFIT1, SIGLEC1, SLC1A2, IL1R2, | 0.883 | 0.873 | 0.892 | 0.939 | 0.893 | 0.985 | 0.950 | 0.883 | 1 |
| OLAH, FAM20A, RETN, MMP8 | | | | | | | | | |
| IFI27, OAS1, IFIT1, SIGLEC1, HERC6, | 0.883 | 0.873 | 0.892 | 0.935 | 0.885 | 0.985 | 0.940 | 0.869 | 1 |
| SLC1A2, FAM20A, RETN, MMP8 | | | | | | | | | |
| IFI27, OAS1, IFI44L, SIGLEC1, ISG15, | 0.883 | 0.872 | 0.893 | 0.931 | 0.876 | 0.985 | 0.943 | 0.871 | 1 |
| HERC6, FAM20A, RETN, MMP8 | | | | | | | | | |
| RSAD2, IFI27, OAS1, IFIT1, ISG15, | 0.882 | 0.873 | 0.892 | 0.929 | 0.875 | 0.982 | 0.935 | 0.867 | 1 |
| IL1R2, OLAH, FAM20A, MMP8 | | | | | | | | | |
| IFI27, IFIT1, SIGLEC1, ISG15, HERC6, | 0.882 | 0.873 | 0.892 | 0.937 | 0.894 | 0.980 | 0.954 | 0.886 | 1 |
| SLC1A2, IL1R2, FAM20A, MMP8 | | | | | | | | | |
| IFI27, IFIT1, IFI44L, SIGLEC1, SLC1A2, | 0.882 | 0.873 | 0.892 | 0.938 | 0.892 | 0.984 | 0.953 | 0.885 | 1 |
| IL1R2, FAM20A, RETN, MMP8 | | | | | | | | | |
| IFI27, OAS1, SIGLEC1, ISG15, HERC6, | 0.882 | 0.872 | 0.892 | 0.935 | 0.887 | 0.983 | 0.936 | 0.866 | 1 |
| SLC1A2, IL1R2, OLAH, FAM20A | | | | | | | | | |
| RSAD2, IFI27, OAS1, IFIT1, IFI44L, | 0.882 | 0.873 | 0.892 | 0.931 | 0.877 | 0.984 | 0.941 | 0.876 | 1 |
| SIGLEC1, SLC1A2, OLAH, FAM20A | | | | | | | | | |
| IFI27, OAS1, IFIT1, IFI44L, HERC6, | 0.882 | 0.873 | 0.892 | 0.931 | 0.879 | 0.983 | 0.928 | 0.855 | 1 |
| IL1R2, OLAH, FAM20A, RETN | | | | | | | | | |
| IFI27, IFI44L, SIGLEC1, ISG15, HERC6, | 0.882 | 0.873 | 0.892 | 0.938 | 0.895 | 0.981 | 0.952 | 0.883 | 1 |
| SLC1A2, IL1R2, FAM20A, MMP8 | | | | | | | | | |
| RSAD2, IFI27, OAS1, IFI44L, ISG15, | 0.882 | 0.872 | 0.893 | 0.930 | 0.877 | 0.983 | 0.928 | 0.856 | 1 |
| IL1R2, OLAH, FAM20A, MMP8 | | | | | | | | | |
| RSAD2, IFI27, SIGLEC1, HERC6, | 0.882 | 0.872 | 0.893 | 0.937 | 0.889 | 0.986 | 0.947 | 0.881 | 1 |
| SLC1A2, OLAH, FAM20A, RETN, MMP8 | | | | | | | | | |
| IFI27, IFIT1, IFI44L, SIGLEC1, SLC1A2, | 0.882 | 0.872 | 0.892 | 0.935 | 0.885 | 0.986 | 0.952 | 0.891 | 1 |
| OLAH, FAM20A, RETN, MMP8 | | | | | | | | | |
| IFI27, OAS1, IFI44L, ISG15, HERC6, | 0.882 | 0.873 | 0.892 | 0.931 | 0.878 | 0.983 | 0.929 | 0.856 | 1 |
| IL1R2, OLAH, FAM20A, RETN | | | | | | | | | |
| IFI27, OAS1, IFIT1, SIGLEC1, HERC6, | 0.882 | 0.872 | 0.892 | 0.939 | 0.895 | 0.984 | 0.936 | 0.869 | 1 |
| SLC1A2, OLAH, FAM20A, RETN | | | | | | | | | |
| RSAD2, IFI27, OAS1, IFIT1, SIGLEC1, | 0.882 | 0.873 | 0.892 | 0.926 | 0.868 | 0.984 | 0.942 | 0.879 | 1 |
| ISG15, OLAH, FAM20A, RETN | | | | | | | | | |
| RSAD2, IFI27, OAS1, SIGLEC1, ISG15, | 0.882 | 0.872 | 0.892 | 0.931 | 0.880 | 0.983 | 0.943 | 0.876 | 1 |
| HERC6, IL1R2, FAM20A, RETN | | | | | | | | | |
| RSAD2, IFI27, OAS1, IFI44L, SIGLEC1, | 0.882 | 0.873 | 0.892 | 0.934 | 0.883 | 0.984 | 0.942 | 0.872 | 1 |
| HERC6, IL1R2, FAM20A, RETN | | | | | | | | | |
| RSAD2, IFI27, OAS1, IFI44L, SIGLEC1, | 0.882 | 0.872 | 0.892 | 0.929 | 0.871 | 0.986 | 0.955 | 0.900 | 1 |
| OLAH, FAM20A, RETN, MMP8 | | | | | | | | | |
| IFI27, IFIT1, SIGLEC1, ISG15, HERC6, | 0.882 | 0.873 | 0.892 | 0.939 | 0.894 | 0.984 | 0.940 | 0.875 | 1 |
| SLC1A2, IL1R2, OLAH, FAM20A | | | | | | | | | |
| IFI27, OAS1, SIGLEC1, ISG15, SLC1A2, | 0.882 | 0.872 | 0.893 | 0.936 | 0.886 | 0.986 | 0.945 | 0.879 | 1 |
| OLAH, FAM20A, RETN, MMP8 | | | | | | | | | |
| RSAD2, IFI27, IFI44L, SIGLEC1, | 0.882 | 0.872 | 0.892 | 0.937 | 0.888 | 0.985 | 0.947 | 0.883 | 1 |
| HERC6, SLC1A2, OLAH, FAM20A, MMP8 | | | | | | | | | |
| IFI27, IFIT1, SIGLEC1, ISG15, SLC1A2, | 0.882 | 0.873 | 0.891 | 0.939 | 0.895 | 0.984 | 0.953 | 0.885 | 1 |
| IL1R2, FAM20A, RETN, MMP8 | | | | | | | | | |
| RSAD2, IFI27, OAS1, IFIT1, IFI44L, | 0.882 | 0.873 | 0.892 | 0.931 | 0.879 | 0.983 | 0.928 | 0.855 | 1 |
| HERC6, OLAH, FAM20A, RETN | | | | | | | | | |
| IFI27, IFI44L, SIGLEC1, SLC1A2, | 0.882 | 0.873 | 0.891 | 0.938 | 0.891 | 0.985 | 0.952 | 0.888 | 1 |
| IL1R2, OLAH, FAM20A, RETN, MMP8 | | | | | | | | | |
| IFI27, OAS1, SIGLEC1, SLC1A2, IL1R2, | 0.882 | 0.872 | 0.892 | 0.935 | 0.887 | 0.984 | 0.950 | 0.886 | 1 |
| OLAH, FAM20A, RETN, MMP8 | | | | | | | | | |
| IFI27, IFIT1, IFI44L, ISG15, IL1R2, | 0.882 | 0.872 | 0.892 | 0.927 | 0.872 | 0.983 | 0.934 | 0.864 | 1 |
| OLAH, FAM20A, RETN, MMP8 | | | | | | | | | |
| IFI27, IFI44L, SIGLEC1, ISG15, | 0.882 | 0.872 | 0.892 | 0.935 | 0.886 | 0.985 | 0.946 | 0.878 | 1 |
| SLC1A2, OLAH, FAM20A, RETN, MMP8 | | | | | | | | | |
| RSAD2, IFI27, OAS1, SIGLEC1, HERC6, | 0.882 | 0.872 | 0.892 | 0.937 | 0.891 | 0.982 | 0.948 | 0.879 | 1 |
| SLC1A2, IL1R2, FAM20A, MMP8 | | | | | | | | | |
| RSAD2, IFI27, OAS1, IFI44L, SIGLEC1, | 0.882 | 0.872 | 0.892 | 0.935 | 0.883 | 0.986 | 0.943 | 0.880 | 1 |
| SLC1A2, OLAH, FAM20A, MMP8 | | | | | | | | | |
| RSAD2, IFI27, IFIT1, SIGLEC1, ISG15, | 0.882 | 0.873 | 0.891 | 0.939 | 0.896 | 0.981 | 0.953 | 0.885 | 1 |
| SLC1A2, IL1R2, FAM20A, MMP8 | | | | | | | | | |
| IFI27, OAS1, IFIT1, IFI44L, HERC6, | 0.882 | 0.872 | 0.892 | 0.930 | 0.878 | 0.983 | 0.929 | 0.849 | 1 |
| IL1R2, FAM20A, RETN, MMP8 | | | | | | | | | |
| RSAD2, IFI27, IFI44L, SIGLEC1, ISG15, | 0.882 | 0.871 | 0.892 | 0.939 | 0.891 | 0.987 | 0.941 | 0.875 | 1 |
| SLC1A2, OLAH, FAM20A, MMP8 | | | | | | | | | |
| RSAD2, IFI27, IFIT1, SIGLEC1, ISG15, | 0.882 | 0.872 | 0.892 | 0.937 | 0.891 | 0.984 | 0.933 | 0.864 | 1 |
| HERC6, SLC1A2, OLAH, FAM20A | | | | | | | | | |
| IFI27, OAS1, SIGLEC1, ISG15, HERC6, | 0.882 | 0.872 | 0.892 | 0.936 | 0.892 | 0.980 | 0.948 | 0.879 | 1 |
| SLC1A2, IL1R2, FAM20A, MMP8 | | | | | | | | | |
| RSAD2, IFI27, IFIT1, SIGLEC1, HERC6, | 0.882 | 0.872 | 0.892 | 0.937 | 0.891 | 0.984 | 0.938 | 0.872 | 1 |
| SLC1A2, IL1R2, OLAH, FAM20A | | | | | | | | | |
| RSAD2, IFI27, IFIT1, IFI44L, SIGLEC1, | 0.882 | 0.873 | 0.891 | 0.936 | 0.891 | 0.981 | 0.953 | 0.885 | 1 |
| SLC1A2, IL1R2, FAM20A, MMP8 | | | | | | | | | |
| IFI27, OAS1, IFIT1, SIGLEC1, SLC1A2, | 0.882 | 0.871 | 0.892 | 0.933 | 0.881 | 0.985 | 0.952 | 0.893 | 1 |
| OLAH, FAM20A, RETN, MMP8 | | | | | | | | | |
| RSAD2, IFI27, IFIT1, IFI44L, SIGLEC1, | 0.882 | 0.872 | 0.891 | 0.927 | 0.870 | 0.984 | 0.946 | 0.887 | 1 |
| ISG15, OLAH, FAM20A, RETN | | | | | | | | | |
| IFI27, OAS1, IFI44L, SIGLEC1, ISG15, | 0.882 | 0.872 | 0.892 | 0.935 | 0.887 | 0.984 | 0.936 | 0.866 | 1 |
| HERC6, SLC1A2, OLAH, FAM20A | | | | | | | | | |
| IFI27, IFIT1, IFI44L, SIGLEC1, ISG15, | 0.882 | 0.873 | 0.891 | 0.935 | 0.885 | 0.985 | 0.939 | 0.873 | 1 |
| SLC1A2, OLAH, FAM20A, RETN | | | | | | | | | |
| IFI27, OAS1, IFIT1, SIGLEC1, HERC6, | 0.882 | 0.873 | 0.891 | 0.927 | 0.874 | 0.981 | 0.958 | 0.909 | 1 |
| IL1R2, OLAH, RETN, MMP8 | | | | | | | | | |
| IFI27, OAS1, IFIT1, IFI44L, SIGLEC1, | 0.882 | 0.873 | 0.891 | 0.937 | 0.893 | 0.981 | 0.941 | 0.872 | 1 |
| HERC6, SLC1A2, IL1R2, FAM20A | | | | | | | | | |
| RSAD2, IFI27, IFI44L, SIGLEC1, | 0.882 | 0.872 | 0.892 | 0.935 | 0.890 | 0.981 | 0.954 | 0.886 | 1 |
| HERC6, SLC1A2, IL1R2, FAM20A, MMP8 | | | | | | | | | |
| RSAD2, IFI27, IFI44L, SIGLEC1, ISG15, | 0.882 | 0.872 | 0.891 | 0.928 | 0.872 | 0.983 | 0.945 | 0.881 | 1 |
| IL1R2, OLAH, FAM20A, RETN | | | | | | | | | |
| RSAD2, IFI27, IFIT1, IFI44L, ISG15, | 0.882 | 0.872 | 0.892 | 0.929 | 0.877 | 0.982 | 0.935 | 0.866 | 1 |
| IL1R2, OLAH, FAM20A, MMP8 | | | | | | | | | |
| IFI27, OAS1, IFIT1, SIGLEC1, ISG15, | 0.882 | 0.872 | 0.891 | 0.938 | 0.895 | 0.981 | 0.951 | 0.882 | 1 |
| SLC1A2, IL1R2, FAM20A, MMP8 | | | | | | | | | |
| IFI27, IFI44L, SIGLEC1, ISG15, HERC6, | 0.882 | 0.872 | 0.891 | 0.937 | 0.890 | 0.983 | 0.940 | 0.871 | 1 |
| SLC1A2, IL1R2, OLAH, FAM20A | | | | | | | | | |
| RSAD2, IFI27, IFIT1, IFI44L, ISG15, | 0.882 | 0.872 | 0.891 | 0.929 | 0.875 | 0.983 | 0.929 | 0.857 | 1 |
| HERC6, IL1R2, OLAH, FAM20A | | | | | | | | | |
| RSAD2, IFI27, IFIT1, SIGLEC1, | 0.882 | 0.872 | 0.892 | 0.936 | 0.886 | 0.986 | 0.949 | 0.886 | 1 |
| SLC1A2, OLAH, FAM20A, RETN, MMP8 | | | | | | | | | |
| IFI27, OAS1, IFIT1, ISG15, HERC6, | 0.882 | 0.872 | 0.891 | 0.930 | 0.878 | 0.982 | 0.928 | 0.855 | 1 |
| IL1R2, OLAH, FAM20A, RETN | | | | | | | | | |
| IFI27, OAS1, IFIT1, IFI44L, SIGLEC1, | 0.882 | 0.873 | 0.891 | 0.929 | 0.875 | 0.983 | 0.949 | 0.886 | 1 |
| ISG15, IL1R2, FAM20A, RETN | | | | | | | | | |
| RSAD2, IFI27, IFI44L, SIGLEC1, ISG15, | 0.882 | 0.871 | 0.892 | 0.931 | 0.875 | 0.987 | 0.950 | 0.891 | 1 |
| OLAH, FAM20A, RETN, MMP8 | | | | | | | | | |
| RSAD2, IFI27, IFIT1, IFI44L, SIGLEC1, | 0.882 | 0.872 | 0.892 | 0.927 | 0.871 | 0.984 | 0.947 | 0.884 | 1 |
| IL1R2, OLAH, FAM20A, RETN | | | | | | | | | |
| IFI27, IFI44L, ISG15, HERC6, SLC1A2, | 0.882 | 0.872 | 0.891 | 0.935 | 0.888 | 0.982 | 0.922 | 0.841 | 1 |
| IL1R2, OLAH, FAM20A, MMP8 | | | | | | | | | |
| RSAD2, IFI27, OAS1, IFI44L, SIGLEC1, | 0.882 | 0.872 | 0.891 | 0.926 | 0.869 | 0.982 | 0.952 | 0.892 | 1 |
| IL1R2, OLAH, FAM20A, RETN | | | | | | | | | |
| IFI27, SIGLEC1, ISG15, HERC6, | 0.882 | 0.871 | 0.892 | 0.934 | 0.884 | 0.984 | 0.948 | 0.880 | 1 |
| SLC1A2, OLAH, FAM20A, RETN, MMP8 | | | | | | | | | |
| IFI27, IFIT1, IFI44L, ISG15, HERC6, | 0.881 | 0.872 | 0.891 | 0.929 | 0.875 | 0.982 | 0.929 | 0.856 | 1 |
| IL1R2, OLAH, FAM20A, RETN | | | | | | | | | |
| RSAD2, IFI27, OAS1, IFIT1, SIGLEC1, | 0.881 | 0.872 | 0.891 | 0.934 | 0.887 | 0.980 | 0.952 | 0.885 | 1 |
| SLC1A2, IL1R2, FAM20A, MMP8 | | | | | | | | | |
| RSAD2, IFI27, IFIT1, SIGLEC1, | 0.881 | 0.872 | 0.891 | 0.938 | 0.892 | 0.985 | 0.952 | 0.883 | 1 |
| SLC1A2, IL1R2, FAM20A, RETN, MMP8 | | | | | | | | | |
| RSAD2, IFI27, IFIT1, IFI44L, SIGLEC1, | 0.881 | 0.872 | 0.891 | 0.935 | 0.885 | 0.985 | 0.939 | 0.873 | 1 |
| ISG15, SLC1A2, OLAH, FAM20A | | | | | | | | | |
| IFI27, IFIT1, IFI44L, SIGLEC1, HERC6, | 0.881 | 0.872 | 0.891 | 0.936 | 0.887 | 0.985 | 0.941 | 0.876 | 1 |
| SLC1A2, OLAH, FAM20A, RETN | | | | | | | | | |
| RSAD2, IFI27, OAS1, SIGLEC1, HERC6, | 0.881 | 0.871 | 0.891 | 0.932 | 0.882 | 0.982 | 0.936 | 0.867 | 1 |
| SLC1A2, IL1R2, OLAH, FAM20A | | | | | | | | | |
| IFI27, OAS1, IFIT1, IFI44L, SIGLEC1, | 0.881 | 0.872 | 0.891 | 0.932 | 0.881 | 0.984 | 0.939 | 0.873 | 1 |
| SLC1A2, IL1R2, OLAH, FAM20A | | | | | | | | | |
| IFI27, IFI44L, ISG15, SLC1A2, IL1R2, | 0.881 | 0.872 | 0.891 | 0.935 | 0.887 | 0.983 | 0.922 | 0.841 | 1 |
| OLAH, FAM20A, RETN, MMP8 | | | | | | | | | |
| IFI27, OAS1, SIGLEC1, ISG15, SLC1A2, | 0.881 | 0.872 | 0.891 | 0.937 | 0.892 | 0.982 | 0.948 | 0.877 | 1 |
| IL1R2, FAM20A, RETN, MMP8 | | | | | | | | | |
| IFI27, IFIT1, SIGLEC1, ISG15, SLC1A2, | 0.881 | 0.872 | 0.891 | 0.936 | 0.888 | 0.984 | 0.936 | 0.868 | 1 |
| IL1R2, OLAH, FAM20A, RETN | | | | | | | | | |
| IFI27, OAS1, IFIT1, IFI44L, SIGLEC1, | 0.881 | 0.872 | 0.891 | 0.930 | 0.877 | 0.984 | 0.940 | 0.875 | 1 |
| SLC1A2, OLAH, FAM20A, RETN | | | | | | | | | |
| RSAD2, IFI27, SIGLEC1, ISG15, | 0.881 | 0.871 | 0.891 | 0.937 | 0.891 | 0.983 | 0.949 | 0.879 | 1 |
| SLC1A2, IL1R2, FAM20A, RETN, MMP8 | | | | | | | | | |
| RSAD2, IFI27, IFI44L, ISG15, HERC6, | 0.881 | 0.871 | 0.892 | 0.929 | 0.874 | 0.984 | 0.930 | 0.859 | 1 |
| OLAH, FAM20A, RETN, MMP8 | | | | | | | | | |
| IFI27, IFI44L, SIGLEC1, ISG15, | 0.881 | 0.871 | 0.891 | 0.938 | 0.892 | 0.984 | 0.950 | 0.879 | 1 |
| SLC1A2, IL1R2, FAM20A, RETN, MMP8 | | | | | | | | | |
| RSAD2, IFI27, OAS1, IFIT1, ISG15, | 0.881 | 0.872 | 0.891 | 0.930 | 0.878 | 0.983 | 0.934 | 0.864 | 1 |
| HERC6, OLAH, FAM20A, RETN | | | | | | | | | |
| IFI27, OAS1, IFIT1, IFI44L, SIGLEC1, | 0.881 | 0.872 | 0.890 | 0.933 | 0.884 | 0.981 | 0.936 | 0.865 | 1 |
| HERC6, SLC1A2, FAM20A, RETN | | | | | | | | | |
| RSAD2, IFI27, OAS1, SIGLEC1, ISG15, | 0.881 | 0.871 | 0.891 | 0.935 | 0.887 | 0.984 | 0.935 | 0.864 | 1 |
| HERC6, SLC1A2, OLAH, FAM20A | | | | | | | | | |
| RSAD2, IFI27, OAS1, HERC6, SLC1A2, | 0.881 | 0.871 | 0.891 | 0.935 | 0.889 | 0.982 | 0.918 | 0.835 | 1 |
| IL1R2, OLAH, FAM20A, MMP8 | | | | | | | | | |
| RSAD2, IFI27, SIGLEC1, ISG15, HERC6, | 0.881 | 0.871 | 0.891 | 0.935 | 0.888 | 0.983 | 0.938 | 0.870 | 1 |
| SLC1A2, IL1R2, OLAH, FAM20A | | | | | | | | | |
| RSAD2, IFI27, OAS1, IFI44L, SIGLEC1, | 0.881 | 0.871 | 0.891 | 0.931 | 0.877 | 0.986 | 0.942 | 0.869 | 1 |
| HERC6, FAM20A, RETN, MMP8 | | | | | | | | | |
| IFI27, OAS1, IFIT1, IFI44L, SIGLEC1, | 0.881 | 0.872 | 0.890 | 0.926 | 0.872 | 0.980 | 0.950 | 0.897 | 1 |
| HERC6, OLAH, RETN, MMP8 | | | | | | | | | |
| IFI27, IFIT1, IFI44L, ISG15, SLC1A2, | 0.881 | 0.872 | 0.890 | 0.934 | 0.886 | 0.982 | 0.924 | 0.846 | 1 |
| IL1R2, OLAH, FAM20A, MMP8 | | | | | | | | | |
| RSAD2, IFI27, OAS1, IFI44L, SIGLEC1, | 0.881 | 0.871 | 0.890 | 0.924 | 0.865 | 0.982 | 0.948 | 0.888 | 1 |
| ISG15, OLAH, FAM20A, RETN | | | | | | | | | |
| IFI27, OAS1, IFIT1, ISG15, IL1R2, | 0.881 | 0.871 | 0.891 | 0.927 | 0.872 | 0.983 | 0.934 | 0.864 | 1 |
| OLAH, FAM20A, RETN, MMP8 | | | | | | | | | |
| RSAD2, IFI27, OAS1, IFIT1, SIGLEC1, | 0.881 | 0.871 | 0.890 | 0.933 | 0.882 | 0.984 | 0.941 | 0.877 | 1 |
| ISG15, SLC1A2, OLAH, FAM20A | | | | | | | | | |
| RSAD2, IFI27, OAS1, IFI44L, SIGLEC1, | 0.881 | 0.871 | 0.891 | 0.929 | 0.875 | 0.983 | 0.939 | 0.866 | 1 |
| ISG15, HERC6, FAM20A, RETN | | | | | | | | | |
| RSAD2, IFI27, OAS1, ISG15, HERC6, | 0.881 | 0.871 | 0.890 | 0.931 | 0.879 | 0.982 | 0.928 | 0.855 | 1 |
| IL1R2, OLAH, FAM20A, RETN | | | | | | | | | |
| RSAD2, IFI27, OAS1, IFI44L, HERC6, | 0.881 | 0.871 | 0.891 | 0.931 | 0.879 | 0.983 | 0.929 | 0.856 | 1 |
| IL1R2, OLAH, FAM20A, RETN | | | | | | | | | |
| IFI27, IFI44L, SIGLEC1, ISG15, | 0.881 | 0.871 | 0.890 | 0.936 | 0.889 | 0.983 | 0.941 | 0.874 | 1 |
| SLC1A2, IL1R2, OLAH, FAM20A, RETN | | | | | | | | | |
| IFI27, OAS1, IFI44L, SIGLEC1, ISG15, | 0.881 | 0.871 | 0.891 | 0.939 | 0.896 | 0.981 | 0.950 | 0.880 | 1 |
| SLC1A2, IL1R2, FAM20A, MMP8 | | | | | | | | | |
| RSAD2, IFI27, IFIT1, ISG15, HERC6, | 0.881 | 0.871 | 0.890 | 0.929 | 0.876 | 0.981 | 0.926 | 0.851 | 1 |
| IL1R2, OLAH, FAM20A, RETN | | | | | | | | | |
| RSAD2, IFI27, OAS1, IFI44L, SIGLEC1, | 0.881 | 0.871 | 0.890 | 0.933 | 0.882 | 0.984 | 0.941 | 0.874 | 1 |
| ISG15, SLC1A2, OLAH, FAM20A | | | | | | | | | |
| RSAD2, IFI27, IFI44L, SIGLEC1, | 0.881 | 0.871 | 0.891 | 0.937 | 0.889 | 0.984 | 0.949 | 0.879 | 1 |
| SLC1A2, IL1R2, FAM20A, RETN, MMP8 | | | | | | | | | |
| IFI27, OAS1, IFIT1, IFI44L, HERC6, | 0.881 | 0.870 | 0.891 | 0.931 | 0.880 | 0.982 | 0.923 | 0.842 | 1 |
| SLC1A2, OLAH, FAM20A, MMP8 | | | | | | | | | |
| RSAD2, IFI27, OAS1, IFIT1, HERC6, | 0.881 | 0.871 | 0.890 | 0.931 | 0.879 | 0.982 | 0.928 | 0.853 | 1 |
| IL1R2, OLAH, FAM20A, RETN | | | | | | | | | |
| IFI27, OAS1, IFI44L, SIGLEC1, SLC1A2, | 0.881 | 0.871 | 0.890 | 0.937 | 0.892 | 0.983 | 0.948 | 0.877 | 1 |
| IL1R2, FAM20A, RETN, MMP8 | | | | | | | | | |
| RSAD2, IFI27, OAS1, SIGLEC1, | 0.881 | 0.870 | 0.891 | 0.933 | 0.881 | 0.985 | 0.950 | 0.889 | 1 |
| SLC1A2, OLAH, FAM20A, RETN, MMP8 | | | | | | | | | |
| RSAD2, IFI27, IFIT1, IFI44L, ISG15, | 0.881 | 0.871 | 0.890 | 0.931 | 0.879 | 0.983 | 0.928 | 0.857 | 0.999 |
| HERC6, OLAH, FAM20A, RETN | | | | | | | | | |
| RSAD2, IFI27, IFIT1, IFI44L, HERC6, | 0.881 | 0.871 | 0.890 | 0.931 | 0.879 | 0.983 | 0.928 | 0.856 | 1 |
| IL1R2, OLAH, FAM20A, RETN | | | | | | | | | |
| RSAD2, IFI27, OAS1, IFIT1, IL1R2, | 0.881 | 0.871 | 0.890 | 0.929 | 0.875 | 0.983 | 0.933 | 0.863 | 1 |
| OLAH, FAM20A, RETN, MMP8 | | | | | | | | | |
| IFI27, OAS1, SIGLEC1, ISG15, HERC6, | 0.881 | 0.870 | 0.891 | 0.935 | 0.887 | 0.983 | 0.935 | 0.864 | 1 |
| SLC1A2, OLAH, FAM20A, RETN | | | | | | | | | |
| IFI27, OAS1, IFI44L, ISG15, HERC6, | 0.881 | 0.870 | 0.891 | 0.932 | 0.880 | 0.984 | 0.930 | 0.849 | 1 |
| IL1R2, FAM20A, RETN, MMP8 | | | | | | | | | |
| IFI27, OAS1, IFIT1, SIGLEC1, ISG15, | 0.881 | 0.871 | 0.890 | 0.926 | 0.872 | 0.980 | 0.955 | 0.905 | 1 |
| HERC6, OLAH, RETN, MMP8 | | | | | | | | | |
| IFI27, OAS1, IFIT1, SIGLEC1, HERC6, | 0.880 | 0.871 | 0.890 | 0.935 | 0.889 | 0.980 | 0.938 | 0.866 | 1 |
| SLC1A2, IL1R2, FAM20A, RETN | | | | | | | | | |
| IFI27, OAS1, IFIT1, SIGLEC1, ISG15, | 0.880 | 0.871 | 0.890 | 0.934 | 0.884 | 0.985 | 0.939 | 0.871 | 1 |
| SLC1A2, OLAH, FAM20A, RETN | | | | | | | | | |
| RSAD2, IFI27, IFI44L, ISG15, HERC6, | 0.880 | 0.870 | 0.891 | 0.930 | 0.878 | 0.983 | 0.930 | 0.860 | 1 |
| IL1R2, OLAH, FAM20A, RETN | | | | | | | | | |
| IFI27, OAS1, IFIT1, SIGLEC1, ISG15, | 0.880 | 0.871 | 0.890 | 0.937 | 0.893 | 0.981 | 0.943 | 0.874 | 1 |
| HERC6, SLC1A2, IL1R2, FAM20A | | | | | | | | | |
| IFI27, IFIT1, IFI44L, SIGLEC1, ISG15, | 0.880 | 0.871 | 0.890 | 0.935 | 0.889 | 0.981 | 0.949 | 0.880 | 1 |
| HERC6, SLC1A2, IL1R2, FAM20A | | | | | | | | | |
| IFI27, OAS1, IFIT1, SIGLEC1, ISG15, | 0.880 | 0.871 | 0.890 | 0.936 | 0.888 | 0.984 | 0.937 | 0.869 | 1 |
| SLC1A2, IL1R2, OLAH, FAM20A | | | | | | | | | |
| RSAD2, IFI27, IFIT1, SIGLEC1, ISG15, | 0.880 | 0.871 | 0.889 | 0.936 | 0.888 | 0.984 | 0.937 | 0.869 | 1 |
| SLC1A2, IL1R2, OLAH, FAM20A | | | | | | | | | |
| RSAD2, IFI27, IFI44L, HERC6, SLC1A2, | 0.880 | 0.870 | 0.891 | 0.935 | 0.888 | 0.983 | 0.928 | 0.853 | 1 |
| IL1R2, OLAH, FAM20A, MMP8 | | | | | | | | | |
| RSAD2, IFI27, IFI44L, SIGLEC1, ISG15, | 0.880 | 0.870 | 0.890 | 0.933 | 0.882 | 0.985 | 0.943 | 0.875 | 1 |
| HERC6, IL1R2, FAM20A, RETN | | | | | | | | | |
| RSAD2, IFI27, OAS1, IFIT1, IFI44L, | 0.880 | 0.871 | 0.889 | 0.929 | 0.875 | 0.982 | 0.949 | 0.886 | 1 |
| SIGLEC1, ISG15, IL1R2, FAM20A | | | | | | | | | |
| RSAD2, IFI27, OAS1, IFIT1, SIGLEC1, | 0.880 | 0.871 | 0.889 | 0.933 | 0.883 | 0.984 | 0.943 | 0.878 | 1 |
| SLC1A2, IL1R2, OLAH, FAM20A | | | | | | | | | |
| IFI27, OAS1, IFIT1, HERC6, SLC1A2, | 0.880 | 0.870 | 0.890 | 0.933 | 0.885 | 0.981 | 0.921 | 0.838 | 1 |
| IL1R2, OLAH, FAM20A, MMP8 | | | | | | | | | |
| IFI27, IFIT1, IFI44L, SIGLEC1, SLC1A2, | 0.880 | 0.871 | 0.890 | 0.932 | 0.881 | 0.983 | 0.942 | 0.878 | 1 |
| IL1R2, OLAH, FAM20A, RETN | | | | | | | | | |
| IFI27, OAS1, IFI44L, SIGLEC1, HERC6, | 0.880 | 0.871 | 0.889 | 0.927 | 0.875 | 0.980 | 0.951 | 0.900 | 1 |
| IL1R2, OLAH, RETN, MMP8 | | | | | | | | | |
| RSAD2, IFI27, OAS1, IFI44L, ISG15, | 0.880 | 0.870 | 0.890 | 0.930 | 0.877 | 0.982 | 0.933 | 0.862 | 1 |
| HERC6, OLAH, FAM20A, RETN | | | | | | | | | |
| RSAD2, IFI27, IFI44L, SIGLEC1, ISG15, | 0.880 | 0.870 | 0.890 | 0.930 | 0.875 | 0.984 | 0.946 | 0.872 | 1 |
| HERC6, FAM20A, RETN, MMP8 | | | | | | | | | |
| RSAD2, IFI27, OAS1, IFIT1, IFI44L, | 0.880 | 0.871 | 0.890 | 0.928 | 0.877 | 0.979 | 0.930 | 0.849 | 1 |
| HERC6, IL1R2, FAM20A, MMP8 | | | | | | | | | |
| IFI27, IFIT1, SIGLEC1, ISG15, HERC6, | 0.880 | 0.870 | 0.890 | 0.938 | 0.893 | 0.984 | 0.936 | 0.868 | 1 |
| SLC1A2, OLAH, FAM20A, RETN | | | | | | | | | |
| RSAD2, IFI27, IFIT1, IFI44L, SIGLEC1, | 0.880 | 0.871 | 0.890 | 0.931 | 0.879 | 0.983 | 0.942 | 0.878 | 1 |
| SLC1A2, IL1R2, OLAH, FAM20A | | | | | | | | | |
| IFI27, OAS1, IFIT1, IFI44L, IL1R2, | 0.880 | 0.870 | 0.890 | 0.928 | 0.874 | 0.983 | 0.933 | 0.863 | 1 |
| OLAH, FAM20A, RETN, MMP8 | | | | | | | | | |
| IFI27, OAS1, IFI44L, SIGLEC1, HERC6, | 0.880 | 0.870 | 0.890 | 0.933 | 0.883 | 0.982 | 0.937 | 0.868 | 1 |
| SLC1A2, IL1R2, OLAH, FAM20A | | | | | | | | | |
| RSAD2, IFI27, IFIT1, HERC6, SLC1A2, | 0.880 | 0.870 | 0.890 | 0.933 | 0.884 | 0.981 | 0.927 | 0.851 | 1 |
| IL1R2, OLAH, FAM20A, MMP8 | | | | | | | | | |
| IFI27, IFIT1, IFI44L, ISG15, HERC6, | 0.880 | 0.870 | 0.890 | 0.931 | 0.881 | 0.982 | 0.922 | 0.841 | 1 |
| SLC1A2, OLAH, FAM20A, MMP8 | | | | | | | | | |
| RSAD2, IFI27, OAS1, ISG15, IL1R2, | 0.880 | 0.870 | 0.890 | 0.928 | 0.874 | 0.983 | 0.930 | 0.860 | 1 |
| OLAH, FAM20A, RETN, MMP8 | | | | | | | | | |
| RSAD2, IFI27, OAS1, SIGLEC1, ISG15, | 0.880 | 0.870 | 0.890 | 0.933 | 0.883 | 0.983 | 0.939 | 0.869 | 1 |
| SLC1A2, IL1R2, OLAH, FAM20A | | | | | | | | | |
| RSAD2, IFI27, IFI44L, SIGLEC1, ISG15, | 0.880 | 0.870 | 0.890 | 0.939 | 0.897 | 0.982 | 0.951 | 0.882 | 1 |
| SLC1A2, IL1R2, FAM20A, MMP8 | | | | | | | | | |
| RSAD2, IFI27, IFI44L, ISG15, SLC1A2, | 0.880 | 0.870 | 0.890 | 0.935 | 0.887 | 0.983 | 0.924 | 0.847 | 1 |
| IL1R2, OLAH, FAM20A, MMP8 | | | | | | | | | |
| RSAD2, IFI27, OAS1, IFI44L, SIGLEC1, | 0.880 | 0.870 | 0.890 | 0.932 | 0.881 | 0.982 | 0.937 | 0.869 | 1 |
| HERC6, SLC1A2, OLAH, FAM20A | | | | | | | | | |
| RSAD2, IFI27, OAS1, SIGLEC1, | 0.880 | 0.870 | 0.890 | 0.936 | 0.890 | 0.982 | 0.950 | 0.882 | 1 |
| SLC1A2, IL1R2, FAM20A, RETN, MMP8 | | | | | | | | | |
| IFI27, OAS1, IFI44L, ISG15, IL1R2, | 0.880 | 0.869 | 0.890 | 0.928 | 0.873 | 0.983 | 0.933 | 0.862 | 1 |
| OLAH, FAM20A, RETN, MMP8 | | | | | | | | | |
| RSAD2, IFI27, HERC6, SLC1A2, IL1R2, | 0.880 | 0.870 | 0.890 | 0.935 | 0.887 | 0.983 | 0.925 | 0.845 | 1 |
| OLAH, FAM20A, RETN, MMP8 | | | | | | | | | |
| IFI27, OAS1, IFIT1, SIGLEC1, ISG15, | 0.880 | 0.871 | 0.889 | 0.924 | 0.871 | 0.978 | 0.947 | 0.892 | 1 |
| HERC6, IL1R2, OLAH, RETN | | | | | | | | | |
| IFI27, IFI44L, SIGLEC1, HERC6, | 0.880 | 0.870 | 0.890 | 0.934 | 0.886 | 0.983 | 0.955 | 0.887 | 1 |
| SLC1A2, IL1R2, FAM20A, RETN, MMP8 | | | | | | | | | |
| RSAD2, IFI27, OAS1, SIGLEC1, ISG15, | 0.880 | 0.870 | 0.890 | 0.937 | 0.893 | 0.980 | 0.949 | 0.880 | 1 |
| SLC1A2, IL1R2, FAM20A, MMP8 | | | | | | | | | |
| IFI27, OAS1, IFI44L, SIGLEC1, ISG15, | 0.880 | 0.870 | 0.890 | 0.934 | 0.886 | 0.983 | 0.941 | 0.874 | 1 |
| SLC1A2, IL1R2, OLAH, FAM20A | | | | | | | | | |
| RSAD2, IFI27, OAS1, IFIT1, SIGLEC1, | 0.880 | 0.870 | 0.889 | 0.927 | 0.873 | 0.981 | 0.954 | 0.903 | 1 |
| HERC6, OLAH, RETN, MMP8 | | | | | | | | | |
| IFI27, OAS1, IFI44L, HERC6, SLC1A2, | 0.880 | 0.869 | 0.890 | 0.934 | 0.886 | 0.983 | 0.923 | 0.842 | 1 |
| IL1R2, OLAH, FAM20A, MMP8 | | | | | | | | | |
| RSAD2, IFI27, OAS1, IFI44L, SIGLEC1, | 0.880 | 0.870 | 0.890 | 0.936 | 0.891 | 0.982 | 0.949 | 0.882 | 1 |
| SLC1A2, IL1R2, FAM20A, MMP8 | | | | | | | | | |
| RSAD2, IFI27, OAS1, SIGLEC1, HERC6, | 0.880 | 0.870 | 0.889 | 0.926 | 0.872 | 0.981 | 0.954 | 0.903 | 1 |
| IL1R2, OLAH, RETN, MMP8 | | | | | | | | | |
| IFI27, OAS1, IFIT1, ISG15, HERC6, | 0.880 | 0.870 | 0.890 | 0.931 | 0.879 | 0.984 | 0.928 | 0.847 | 1 |
| IL1R2, FAM20A, RETN, MMP8 | | | | | | | | | |
| IFI27, OAS1, IFI44L, SIGLEC1, ISG15, | 0.880 | 0.870 | 0.889 | 0.932 | 0.881 | 0.983 | 0.938 | 0.870 | 1 |
| SLC1A2, OLAH, FAM20A, RETN | | | | | | | | | |
| RSAD2, IFI27, ISG15, HERC6, SLC1A2, | 0.880 | 0.870 | 0.889 | 0.933 | 0.886 | 0.980 | 0.920 | 0.838 | 1 |
| IL1R2, OLAH, FAM20A, MMP8 | | | | | | | | | |
| RSAD2, IFI27, IFIT1, ISG15, IL1R2, | 0.880 | 0.870 | 0.890 | 0.928 | 0.872 | 0.983 | 0.932 | 0.860 | 1 |
| OLAH, FAM20A, RETN, MMP8 | | | | | | | | | |
| IFI27, IFIT1, IFI44L, HERC6, SLC1A2, | 0.880 | 0.870 | 0.889 | 0.934 | 0.885 | 0.982 | 0.925 | 0.845 | 1 |
| IL1R2, OLAH, FAM20A, MMP8 | | | | | | | | | |
| RSAD2, IFI27, OAS1, IFIT1, IFI44L, | 0.880 | 0.871 | 0.888 | 0.921 | 0.864 | 0.978 | 0.942 | 0.881 | 1 |
| SIGLEC1, ISG15, HERC6, RETN | | | | | | | | | |
| RSAD2, IFI27, OAS1, IFIT1, IFI44L, | 0.880 | 0.870 | 0.890 | 0.932 | 0.882 | 0.982 | 0.921 | 0.838 | 1 |
| HERC6, SLC1A2, OLAH, FAM20A | | | | | | | | | |
| RSAD2, IFI27, OAS1, IFIT1, HERC6, | 0.880 | 0.869 | 0.890 | 0.929 | 0.877 | 0.981 | 0.923 | 0.843 | 1 |
| SLC1A2, OLAH, FAM20A, MMP8 | | | | | | | | | |
| RSAD2, IFI27, IFIT1, SIGLEC1, ISG15, | 0.880 | 0.870 | 0.889 | 0.934 | 0.884 | 0.985 | 0.938 | 0.871 | 1 |
| SLC1A2, OLAH, FAM20A, RETN | | | | | | | | | |
| RSAD2, IFI27, IFI44L, SIGLEC1, ISG15, | 0.880 | 0.869 | 0.890 | 0.933 | 0.883 | 0.982 | 0.934 | 0.864 | 1 |
| HERC6, SLC1A2, OLAH, FAM20A | | | | | | | | | |
| IFI27, OAS1, IFI44L, ISG15, HERC6, | 0.880 | 0.869 | 0.890 | 0.931 | 0.881 | 0.982 | 0.922 | 0.841 | 1 |
| SLC1A2, OLAH, FAM20A, MMP8 | | | | | | | | | |
| RSAD2, IFI27, OAS1, IFI44L, SIGLEC1, | 0.880 | 0.870 | 0.889 | 0.928 | 0.874 | 0.981 | 0.948 | 0.892 | 1 |
| HERC6, OLAH, RETN, MMP8 | | | | | | | | | |
| IFI27, OAS1, IFIT1, IFI44L, ISG15, | 0.880 | 0.869 | 0.890 | 0.930 | 0.877 | 0.982 | 0.923 | 0.846 | 1.000 |
| SLC1A2, OLAH, FAM20A, MMP8 | | | | | | | | | |
| RSAD2, IFI27, OAS1, IFIT1, SIGLEC1, | 0.880 | 0.870 | 0.889 | 0.937 | 0.893 | 0.980 | 0.941 | 0.872 | 1 |
| HERC6, SLC1A2, IL1R2, FAM20A | | | | | | | | | |
| IFI27, OAS1, SIGLEC1, ISG15, SLC1A2, | 0.879 | 0.870 | 0.889 | 0.934 | 0.884 | 0.984 | 0.938 | 0.870 | 1 |
| IL1R2, OLAH, FAM20A, RETN | | | | | | | | | |
| IFI27, OAS1, IFIT1, SIGLEC1, SLC1A2, | 0.879 | 0.870 | 0.889 | 0.933 | 0.882 | 0.983 | 0.941 | 0.875 | 1 |
| IL1R2, OLAH, FAM20A, RETN | | | | | | | | | |
| RSAD2, IFI27, IFIT1, IFI44L, IL1R2, | 0.879 | 0.869 | 0.890 | 0.929 | 0.874 | 0.984 | 0.930 | 0.859 | 1 |
| OLAH, FAM20A, RETN, MMP8 | | | | | | | | | |
| IFI27, OAS1, SIGLEC1, ISG15, HERC6, | 0.879 | 0.870 | 0.889 | 0.927 | 0.873 | 0.981 | 0.955 | 0.905 | 1 |
| IL1R2, OLAH, RETN, MMP8 | | | | | | | | | |
| RSAD2, IFI27, IFIT1, IFI44L, HERC6, | 0.879 | 0.869 | 0.890 | 0.934 | 0.884 | 0.983 | 0.925 | 0.848 | 1 |
| SLC1A2, OLAH, FAM20A, MMP8 | | | | | | | | | |
| IFI27, OAS1, IFIT1, IFI44L, ISG15, | 0.879 | 0.870 | 0.889 | 0.932 | 0.881 | 0.982 | 0.927 | 0.844 | 1 |
| HERC6, IL1R2, FAM20A, RETN | | | | | | | | | |
| IFI27, OAS1, IFIT1, HERC6, SLC1A2, | 0.879 | 0.869 | 0.890 | 0.933 | 0.883 | 0.983 | 0.922 | 0.840 | 1 |
| OLAH, FAM20A, RETN, MMP8 | | | | | | | | | |
| IFI27, OAS1, IFI44L, SIGLEC1, HERC6, | 0.879 | 0.869 | 0.889 | 0.934 | 0.886 | 0.983 | 0.936 | 0.866 | 1 |
| SLC1A2, OLAH, FAM20A, RETN | | | | | | | | | |
| RSAD2, IFI27, IFIT1, SIGLEC1, HERC6, | 0.879 | 0.869 | 0.889 | 0.938 | 0.892 | 0.985 | 0.938 | 0.872 | 1 |
| SLC1A2, OLAH, FAM20A, RETN | | | | | | | | | |
| RSAD2, IFI27, OAS1, IFIT1, SIGLEC1, | 0.879 | 0.870 | 0.888 | 0.928 | 0.873 | 0.982 | 0.950 | 0.889 | 1 |
| ISG15, IL1R2, FAM20A, RETN | | | | | | | | | |
| RSAD2, IFI27, OAS1, IFI44L, HERC6, | 0.879 | 0.869 | 0.890 | 0.931 | 0.881 | 0.982 | 0.921 | 0.841 | 1 |
| SLC1A2, OLAH, FAM20A, MMP8 | | | | | | | | | |
| IFI27, SIGLEC1, ISG15, HERC6, | 0.879 | 0.869 | 0.889 | 0.937 | 0.891 | 0.982 | 0.951 | 0.880 | 1 |
| SLC1A2, IL1R2, FAM20A, RETN, MMP8 | | | | | | | | | |
| RSAD2, IFI27, OAS1, HERC6, SLC1A2, | 0.879 | 0.869 | 0.889 | 0.934 | 0.884 | 0.983 | 0.922 | 0.838 | 1 |
| OLAH, FAM20A, RETN, MMP8 | | | | | | | | | |
| IFI27, OAS1, IFI44L, SIGLEC1, ISG15, | 0.879 | 0.870 | 0.889 | 0.935 | 0.889 | 0.980 | 0.945 | 0.876 | 1 |
| HERC6, SLC1A2, IL1R2, FAM20A | | | | | | | | | |
| RSAD2, IFI27, OAS1, ISG15, HERC6, | 0.879 | 0.869 | 0.889 | 0.931 | 0.882 | 0.980 | 0.918 | 0.835 | 1 |
| SLC1A2, OLAH, FAM20A, MMP8 | | | | | | | | | |
| RSAD2, IFI27, IFIT1, SIGLEC1, HERC6, | 0.879 | 0.870 | 0.889 | 0.930 | 0.878 | 0.983 | 0.942 | 0.869 | 1 |
| SLC1A2, FAM20A, RETN, MMP8 | | | | | | | | | |
| RSAD2, IFI27, OAS1, IFIT1, IFI44L, | 0.879 | 0.869 | 0.889 | 0.928 | 0.873 | 0.982 | 0.950 | 0.888 | 1 |
| SIGLEC1, IL1R2, FAM20A, RETN | | | | | | | | | |
| IFI27, OAS1, ISG15, HERC6, SLC1A2, | 0.879 | 0.869 | 0.889 | 0.935 | 0.889 | 0.981 | 0.922 | 0.841 | 1 |
| IL1R2, OLAH, FAM20A, MMP8 | | | | | | | | | |
| RSAD2, IFI27, OAS1, IFIT1, HERC6, | 0.879 | 0.869 | 0.889 | 0.930 | 0.877 | 0.983 | 0.928 | 0.847 | 1 |
| IL1R2, FAM20A, RETN, MMP8 | | | | | | | | | |
| RSAD2, IFI27, IFIT1, IFI44L, SIGLEC1, | 0.879 | 0.870 | 0.888 | 0.926 | 0.871 | 0.980 | 0.951 | 0.900 | 1 |
| HERC6, OLAH, RETN, MMP8 | | | | | | | | | |
| IFI27, IFI44L, SIGLEC1, ISG15, HERC6, | 0.879 | 0.869 | 0.889 | 0.933 | 0.882 | 0.983 | 0.938 | 0.869 | 1 |
| SLC1A2, OLAH, FAM20A, RETN | | | | | | | | | |
| RSAD2, IFI27, IFI44L, SIGLEC1, | 0.879 | 0.870 | 0.888 | 0.927 | 0.874 | 0.980 | 0.952 | 0.903 | 1 |
| HERC6, IL1R2, OLAH, RETN, MMP8 | | | | | | | | | |
| RSAD2, IFI27, OAS1, IFIT1, IFI44L, | 0.879 | 0.869 | 0.890 | 0.924 | 0.866 | 0.983 | 0.937 | 0.873 | 1 |
| ISG15, OLAH, FAM20A, MMP8 | | | | | | | | | |
| RSAD2, IFI27, IFIT1, ISG15, SLC1A2, | 0.879 | 0.869 | 0.889 | 0.933 | 0.885 | 0.981 | 0.925 | 0.847 | 1 |
| IL1R2, OLAH, FAM20A, MMP8 | | | | | | | | | |
| IFI27, OAS1, IFI44L, SIGLEC1, SLC1A2, | 0.879 | 0.868 | 0.890 | 0.933 | 0.880 | 0.985 | 0.952 | 0.893 | 1 |
| OLAH, FAM20A, RETN, MMP8 | | | | | | | | | |
| RSAD2, IFI27, OAS1, ISG15, HERC6, | 0.879 | 0.869 | 0.889 | 0.931 | 0.879 | 0.983 | 0.927 | 0.844 | 1 |
| IL1R2, FAM20A, RETN, MMP8 | | | | | | | | | |
| RSAD2, IFI27, OAS1, IFI44L, ISG15, | 0.879 | 0.869 | 0.889 | 0.929 | 0.878 | 0.980 | 0.926 | 0.840 | 1 |
| HERC6, IL1R2, FAM20A, MMP8 | | | | | | | | | |
| RSAD2, IFI27, IFI44L, ISG15, HERC6, | 0.879 | 0.868 | 0.890 | 0.934 | 0.884 | 0.983 | 0.923 | 0.845 | 1 |
| SLC1A2, OLAH, FAM20A, MMP8 | | | | | | | | | |
| RSAD2, IFI27, IFI44L, SIGLEC1, | 0.879 | 0.869 | 0.889 | 0.935 | 0.884 | 0.986 | 0.948 | 0.884 | 1 |
| SLC1A2, OLAH, FAM20A, RETN, MMP8 | | | | | | | | | |
| RSAD2, IFI27, IFIT1, IFI44L, SLC1A2, | 0.879 | 0.869 | 0.889 | 0.933 | 0.882 | 0.983 | 0.927 | 0.851 | 1 |
| IL1R2, OLAH, FAM20A, MMP8 | | | | | | | | | |
| RSAD2, IFI27, IFIT1, IFI44L, ISG15, | 0.879 | 0.869 | 0.889 | 0.930 | 0.878 | 0.983 | 0.923 | 0.846 | 1 |
| SLC1A2, OLAH, FAM20A, MMP8 | | | | | | | | | |
| RSAD2, IFI27, OAS1, IFIT1, IFI44L, | 0.879 | 0.870 | 0.888 | 0.924 | 0.870 | 0.979 | 0.942 | 0.884 | 1 |
| SIGLEC1, HERC6, OLAH, RETN | | | | | | | | | |
| RSAD2, IFI27, OAS1, IFI44L, IL1R2, | 0.879 | 0.868 | 0.890 | 0.930 | 0.877 | 0.984 | 0.934 | 0.865 | 1 |
| OLAH, FAM20A, RETN, MMP8 | | | | | | | | | |
| RSAD2, IFI27, IFI44L, SIGLEC1, | 0.879 | 0.868 | 0.889 | 0.933 | 0.884 | 0.983 | 0.945 | 0.878 | 1 |
| HERC6, SLC1A2, IL1R2, OLAH, FAM20A | | | | | | | | | |
| RSAD2, IFI27, IFIT1, SIGLEC1, HERC6, | 0.879 | 0.870 | 0.888 | 0.926 | 0.872 | 0.980 | 0.951 | 0.900 | 1 |
| IL1R2, OLAH, RETN, MMP8 | | | | | | | | | |
| OAS1, IFIT1, IFI44L, SIGLEC1, HERC6, | 0.879 | 0.870 | 0.888 | 0.924 | 0.870 | 0.977 | 0.963 | 0.909 | 1 |
| IL1R2, OLAH, FAM20A, MMP8 | | | | | | | | | |
| RSAD2, IFI27, OAS1, IFIT1, SLC1A2, | 0.879 | 0.869 | 0.888 | 0.931 | 0.880 | 0.981 | 0.924 | 0.845 | 1 |
| IL1R2, OLAH, FAM20A, MMP8 | | | | | | | | | |
| IFI27, OAS1, IFI44L, SIGLEC1, ISG15, | 0.879 | 0.870 | 0.888 | 0.926 | 0.872 | 0.980 | 0.951 | 0.900 | 1 |
| HERC6, OLAH, RETN, MMP8 | | | | | | | | | |
| RSAD2, IFI27, IFIT1, IFI44L, SIGLEC1, | 0.879 | 0.869 | 0.888 | 0.936 | 0.890 | 0.981 | 0.946 | 0.876 | 1 |
| HERC6, SLC1A2, IL1R2, FAM20A | | | | | | | | | |
| RSAD2, IFI27, IFIT1, HERC6, SLC1A2, | 0.879 | 0.869 | 0.889 | 0.935 | 0.886 | 0.984 | 0.923 | 0.843 | 1 |
| OLAH, FAM20A, RETN, MMP8 | | | | | | | | | |
| RSAD2, IFI27, OAS1, IFI44L, SIGLEC1, | 0.879 | 0.869 | 0.889 | 0.927 | 0.873 | 0.981 | 0.949 | 0.884 | 1 |
| ISG15, IL1R2, FAM20A, RETN | | | | | | | | | |
| IFI27, OAS1, IFI44L, ISG15, HERC6, | 0.879 | 0.868 | 0.889 | 0.934 | 0.885 | 0.982 | 0.918 | 0.836 | 1 |
| SLC1A2, IL1R2, OLAH, FAM20A | | | | | | | | | |
| IFI27, IFI44L, SIGLEC1, HERC6, | 0.879 | 0.869 | 0.889 | 0.930 | 0.877 | 0.984 | 0.947 | 0.878 | 1 |
| SLC1A2, OLAH, FAM20A, RETN, MMP8 | | | | | | | | | |
| RSAD2, IFI27, SIGLEC1, ISG15, | 0.879 | 0.869 | 0.889 | 0.933 | 0.883 | 0.983 | 0.938 | 0.869 | 1 |
| SLC1A2, IL1R2, OLAH, FAM20A, RETN | | | | | | | | | |
| IFI27, OAS1, IFIT1, IFI44L, ISG15, | 0.879 | 0.868 | 0.889 | 0.934 | 0.886 | 0.983 | 0.918 | 0.836 | 1 |
| HERC6, SLC1A2, OLAH, FAM20A | | | | | | | | | |
| RSAD2, IFI27, OAS1, SIGLEC1, ISG15, | 0.879 | 0.869 | 0.888 | 0.925 | 0.870 | 0.980 | 0.955 | 0.905 | 1 |
| HERC6, OLAH, RETN, MMP8 | | | | | | | | | |
| IFI27, IFIT1, SIGLEC1, HERC6, | 0.879 | 0.869 | 0.889 | 0.936 | 0.888 | 0.984 | 0.941 | 0.875 | 1 |
| SLC1A2, IL1R2, OLAH, FAM20A, RETN | | | | | | | | | |
| RSAD2, IFI27, IFIT1, IFI44L, HERC6, | 0.879 | 0.869 | 0.889 | 0.931 | 0.879 | 0.983 | 0.935 | 0.857 | 1 |
| IL1R2, FAM20A, RETN, MMP8 | | | | | | | | | |
| RSAD2, IFI27, OAS1, IFIT1, ISG15, | 0.879 | 0.869 | 0.888 | 0.927 | 0.873 | 0.981 | 0.925 | 0.849 | 1 |
| SLC1A2, OLAH, FAM20A, MMP8 | | | | | | | | | |
| IFI27, IFIT1, IFI44L, SIGLEC1, HERC6, | 0.879 | 0.870 | 0.888 | 0.925 | 0.871 | 0.979 | 0.953 | 0.904 | 1 |
| IL1R2, OLAH, RETN, MMP8 | | | | | | | | | |
| IFI27, OAS1, SIGLEC1, HERC6, | 0.879 | 0.868 | 0.889 | 0.932 | 0.882 | 0.982 | 0.936 | 0.866 | 1 |
| SLC1A2, IL1R2, OLAH, FAM20A, RETN | | | | | | | | | |
| IFI27, OAS1, IFI44L, ISG15, SLC1A2, | 0.879 | 0.868 | 0.889 | 0.935 | 0.887 | 0.983 | 0.924 | 0.847 | 1 |
| IL1R2, OLAH, FAM20A, MMP8 | | | | | | | | | |
| IFI27, IFIT1, IFI44L, SIGLEC1, HERC6, | 0.879 | 0.869 | 0.888 | 0.937 | 0.891 | 0.982 | 0.946 | 0.876 | 1 |
| SLC1A2, IL1R2, FAM20A, RETN | | | | | | | | | |
| IFI27, OAS1, IFIT1, IFI44L, SIGLEC1, | 0.879 | 0.868 | 0.889 | 0.923 | 0.863 | 0.982 | 0.946 | 0.875 | 1 |
| ISG15, FAM20A, RETN, MMP8 | | | | | | | | | |
| IFI27, SIGLEC1, HERC6, SLC1A2, | 0.879 | 0.868 | 0.889 | 0.936 | 0.886 | 0.985 | 0.948 | 0.880 | 1 |
| IL1R2, OLAH, FAM20A, RETN, MMP8 | | | | | | | | | |
| RSAD2, IFI27, OAS1, IFI44L, HERC6, | 0.879 | 0.868 | 0.889 | 0.930 | 0.878 | 0.983 | 0.932 | 0.850 | 1 |
| IL1R2, FAM20A, RETN, MMP8 | | | | | | | | | |
| IFI27, OAS1, ISG15, HERC6, SLC1A2, | 0.879 | 0.868 | 0.889 | 0.933 | 0.884 | 0.982 | 0.920 | 0.836 | 1 |
| OLAH, FAM20A, RETN, MMP8 | | | | | | | | | |
| IFI27, OAS1, HERC6, SLC1A2, IL1R2, | 0.879 | 0.869 | 0.889 | 0.934 | 0.885 | 0.983 | 0.921 | 0.838 | 1 |
| OLAH, FAM20A, RETN, MMP8 | | | | | | | | | |
| RSAD2, IFI27, OAS1, ISG15, HERC6, | 0.879 | 0.869 | 0.888 | 0.935 | 0.888 | 0.982 | 0.915 | 0.828 | 1 |
| SLC1A2, IL1R2, OLAH, FAM20A | | | | | | | | | |
| RSAD2, IFI27, OAS1, SIGLEC1, ISG15, | 0.879 | 0.869 | 0.888 | 0.931 | 0.879 | 0.984 | 0.940 | 0.873 | 1 |
| SLC1A2, OLAH, FAM20A, RETN | | | | | | | | | |
| RSAD2, IFI27, OAS1, SIGLEC1, HERC6, | 0.879 | 0.869 | 0.888 | 0.933 | 0.883 | 0.983 | 0.935 | 0.864 | 1 |
| SLC1A2, OLAH, FAM20A, RETN | | | | | | | | | |
| RSAD2, OAS1, IFIT1, SIGLEC1, HERC6, | 0.879 | 0.869 | 0.888 | 0.924 | 0.870 | 0.978 | 0.962 | 0.906 | 1 |
| IL1R2, OLAH, FAM20A, MMP8 | | | | | | | | | |
| RSAD2, IFI27, IFI44L, SIGLEC1, ISG15, | 0.879 | 0.868 | 0.889 | 0.936 | 0.888 | 0.983 | 0.940 | 0.872 | 1 |
| SLC1A2, IL1R2, OLAH, FAM20A | | | | | | | | | |
| RSAD2, IFI27, OAS1, IFIT1, IFI44L, | 0.879 | 0.869 | 0.888 | 0.931 | 0.878 | 0.984 | 0.926 | 0.850 | 1 |
| SLC1A2, OLAH, FAM20A, MMP8 | | | | | | | | | |
| IFI27, IFI44L, SIGLEC1, ISG15, HERC6, | 0.879 | 0.870 | 0.887 | 0.927 | 0.874 | 0.980 | 0.952 | 0.902 | 1 |
| IL1R2, OLAH, RETN, MMP8 | | | | | | | | | |
| RSAD2, IFI27, IFIT1, SIGLEC1, ISG15, | 0.878 | 0.869 | 0.888 | 0.936 | 0.891 | 0.981 | 0.948 | 0.879 | 1 |
| HERC6, SLC1A2, IL1R2, FAM20A | | | | | | | | | |
| IFI27, IFIT1, IFI44L, SIGLEC1, HERC6, | 0.878 | 0.869 | 0.888 | 0.929 | 0.875 | 0.982 | 0.950 | 0.878 | 1 |
| SLC1A2, FAM20A, RETN, MMP8 | | | | | | | | | |
| RSAD2, IFI27, IFIT1, IFI44L, SIGLEC1, | 0.878 | 0.869 | 0.888 | 0.928 | 0.873 | 0.983 | 0.948 | 0.883 | 1 |
| ISG15, IL1R2, FAM20A, RETN | | | | | | | | | |
| IFI27, OAS1, IFIT1, ISG15, HERC6, | 0.878 | 0.868 | 0.889 | 0.931 | 0.881 | 0.981 | 0.920 | 0.839 | 1 |
| SLC1A2, OLAH, FAM20A, MMP8 | | | | | | | | | |
| IFI27, OAS1, IFIT1, IFI44L, SIGLEC1, | 0.878 | 0.870 | 0.887 | 0.924 | 0.872 | 0.976 | 0.941 | 0.884 | 0.999 |
| HERC6, IL1R2, OLAH, RETN | | | | | | | | | |
| IFI27, OAS1, IFIT1, IFI44L, SIGLEC1, | 0.878 | 0.869 | 0.887 | 0.920 | 0.863 | 0.978 | 0.943 | 0.886 | 1 |
| ISG15, HERC6, RETN, MMP8 | | | | | | | | | |
| IFI27, OAS1, IFIT1, IFI44L, SIGLEC1, | 0.878 | 0.870 | 0.887 | 0.924 | 0.871 | 0.976 | 0.941 | 0.884 | 0.999 |
| ISG15, HERC6, OLAH, RETN | | | | | | | | | |
| IFI27, OAS1, IFIT1, IFI44L, SIGLEC1, | 0.878 | 0.869 | 0.888 | 0.932 | 0.884 | 0.981 | 0.942 | 0.873 | 1 |
| SLC1A2, IL1R2, FAM20A, RETN | | | | | | | | | |
| RSAD2, IFI27, SIGLEC1, ISG15, HERC6, | 0.878 | 0.868 | 0.888 | 0.931 | 0.881 | 0.982 | 0.936 | 0.867 | 1 |
| SLC1A2, OLAH, FAM20A, RETN | | | | | | | | | |
| RSAD2, IFI27, IFI44L, ISG15, IL1R2, | 0.878 | 0.867 | 0.889 | 0.930 | 0.876 | 0.984 | 0.933 | 0.863 | 1 |
| OLAH, FAM20A, RETN, MMP8 | | | | | | | | | |
| IFI27, OAS1, IFIT1, IFI44L, HERC6, | 0.878 | 0.868 | 0.888 | 0.934 | 0.885 | 0.982 | 0.920 | 0.837 | 1 |
| SLC1A2, IL1R2, OLAH, FAM20A | | | | | | | | | |
| RSAD2, IFI27, IFI44L, HERC6, SLC1A2, | 0.878 | 0.868 | 0.888 | 0.935 | 0.886 | 0.984 | 0.924 | 0.845 | 1 |
| OLAH, FAM20A, RETN, MMP8 | | | | | | | | | |
| RSAD2, IFI27, IFIT1, SIGLEC1, | 0.878 | 0.869 | 0.888 | 0.933 | 0.882 | 0.983 | 0.943 | 0.879 | 1 |
| SLC1A2, IL1R2, OLAH, FAM20A, RETN | | | | | | | | | |
| IFI27, OAS1, IFIT1, IFI44L, ISG15, | 0.878 | 0.868 | 0.888 | 0.929 | 0.879 | 0.980 | 0.929 | 0.846 | 1 |
| HERC6, IL1R2, FAM20A, MMP8 | | | | | | | | | |
| RSAD2, IFI27, OAS1, IFIT1, IFI44L, | 0.878 | 0.869 | 0.888 | 0.928 | 0.877 | 0.979 | 0.925 | 0.841 | 1 |
| ISG15, HERC6, IL1R2, FAM20A | | | | | | | | | |
| RSAD2, IFI27, OAS1, IFI44L, SIGLEC1, | 0.878 | 0.869 | 0.887 | 0.924 | 0.871 | 0.977 | 0.943 | 0.885 | 1 |
| HERC6, IL1R2, OLAH, RETN | | | | | | | | | |
| IFI27, IFIT1, IFI44L, ISG15, HERC6, | 0.878 | 0.868 | 0.888 | 0.931 | 0.878 | 0.983 | 0.935 | 0.856 | 1 |
| IL1R2, FAM20A, RETN, MMP8 | | | | | | | | | |
| IFI27, IFIT1, IFI44L, SIGLEC1, ISG15, | 0.878 | 0.869 | 0.887 | 0.925 | 0.870 | 0.980 | 0.949 | 0.896 | 1 |
| HERC6, OLAH, RETN, MMP8 | | | | | | | | | |
| IFI27, OAS1, IFIT1, SIGLEC1, ISG15, | 0.878 | 0.868 | 0.888 | 0.931 | 0.881 | 0.980 | 0.936 | 0.865 | 1 |
| HERC6, SLC1A2, FAM20A, RETN | | | | | | | | | |
| IFI27, IFIT1, IFI44L, SIGLEC1, ISG15, | 0.878 | 0.869 | 0.887 | 0.934 | 0.887 | 0.980 | 0.943 | 0.874 | 1 |
| SLC1A2, IL1R2, FAM20A, RETN | | | | | | | | | |
| RSAD2, IFI27, OAS1, IFIT1, IFI44L, | 0.878 | 0.869 | 0.888 | 0.920 | 0.860 | 0.980 | 0.942 | 0.872 | 1 |
| SIGLEC1, ISG15, FAM20A, RETN | | | | | | | | | |
| IFI27, OAS1, IFIT1, IFI44L, SIGLEC1, | 0.878 | 0.869 | 0.887 | 0.933 | 0.886 | 0.980 | 0.943 | 0.875 | 1 |
| ISG15, SLC1A2, IL1R2, FAM20A | | | | | | | | | |
| RSAD2, IFI27, OAS1, IFIT1, IFI44L, | 0.878 | 0.868 | 0.888 | 0.924 | 0.867 | 0.980 | 0.930 | 0.851 | 1 |
| ISG15, HERC6, FAM20A, RETN | | | | | | | | | |
| RSAD2, IFI27, IFIT1, ISG15, HERC6, | 0.878 | 0.868 | 0.889 | 0.931 | 0.881 | 0.981 | 0.922 | 0.844 | 1.000 |
| SLC1A2, OLAH, FAM20A, MMP8 | | | | | | | | | |
| RSAD2, IFI27, ISG15, HERC6, SLC1A2, | 0.878 | 0.868 | 0.888 | 0.932 | 0.882 | 0.982 | 0.920 | 0.836 | 1 |
| OLAH, FAM20A, RETN, MMP8 | | | | | | | | | |
| RSAD2, IFI27, OAS1, IFI44L, ISG15, | 0.878 | 0.868 | 0.889 | 0.932 | 0.883 | 0.982 | 0.920 | 0.837 | 1 |
| HERC6, SLC1A2, OLAH, FAM20A | | | | | | | | | |
| RSAD2, IFI27, OAS1, IFIT1, IFI44L, | 0.878 | 0.868 | 0.888 | 0.930 | 0.879 | 0.981 | 0.929 | 0.848 | 1 |
| HERC6, IL1R2, FAM20A, RETN | | | | | | | | | |
| IFI27, OAS1, IFI44L, ISG15, HERC6, | 0.878 | 0.868 | 0.888 | 0.935 | 0.887 | 0.983 | 0.920 | 0.835 | 1 |
| SLC1A2, OLAH, FAM20A, RETN | | | | | | | | | |
| IFI27, IFIT1, ISG15, HERC6, SLC1A2, | 0.878 | 0.868 | 0.888 | 0.934 | 0.886 | 0.981 | 0.924 | 0.845 | 1 |
| IL1R2, OLAH, FAM20A, MMP8 | | | | | | | | | |
| RSAD2, IFI27, IFI44L, SIGLEC1, ISG15, | 0.878 | 0.868 | 0.888 | 0.930 | 0.878 | 0.982 | 0.939 | 0.871 | 1 |
| SLC1A2, OLAH, FAM20A, RETN | | | | | | | | | |
| IFI27, OAS1, IFIT1, IFI44L, HERC6, | 0.878 | 0.868 | 0.888 | 0.934 | 0.886 | 0.982 | 0.923 | 0.842 | 1 |
| SLC1A2, OLAH, FAM20A, RETN | | | | | | | | | |
| RSAD2, IFI27, OAS1, IFIT1, SIGLEC1, | 0.878 | 0.869 | 0.887 | 0.926 | 0.873 | 0.980 | 0.947 | 0.891 | 1 |
| HERC6, IL1R2, OLAH, RETN | | | | | | | | | |
| RSAD2, IFI27, IFIT1, ISG15, HERC6, | 0.878 | 0.868 | 0.888 | 0.931 | 0.879 | 0.984 | 0.935 | 0.857 | 1 |
| IL1R2, FAM20A, RETN, MMP8 | | | | | | | | | |
| OAS1, IFIT1, SIGLEC1, ISG15, HERC6, | 0.878 | 0.869 | 0.887 | 0.922 | 0.867 | 0.977 | 0.962 | 0.906 | 1 |
| IL1R2, OLAH, FAM20A, MMP8 | | | | | | | | | |
| RSAD2, IFI27, IFIT1, SIGLEC1, ISG15, | 0.878 | 0.869 | 0.887 | 0.924 | 0.869 | 0.979 | 0.951 | 0.900 | 1 |
| HERC6, OLAH, RETN, MMP8 | | | | | | | | | |
| RSAD2, IFI27, OAS1, ISG15, SLC1A2, | 0.878 | 0.868 | 0.888 | 0.936 | 0.890 | 0.982 | 0.917 | 0.834 | 1 |
| IL1R2, OLAH, FAM20A, MMP8 | | | | | | | | | |
| IFI27, OAS1, IFIT1, ISG15, SLC1A2, | 0.878 | 0.868 | 0.888 | 0.934 | 0.886 | 0.981 | 0.925 | 0.847 | 1 |
| IL1R2, OLAH, FAM20A, MMP8 | | | | | | | | | |
| RSAD2, IFI27, OAS1, SIGLEC1, ISG15, | 0.878 | 0.868 | 0.888 | 0.935 | 0.890 | 0.980 | 0.943 | 0.874 | 1 |
| HERC6, SLC1A2, IL1R2, FAM20A | | | | | | | | | |
| RSAD2, IFI27, SIGLEC1, HERC6, | 0.878 | 0.868 | 0.888 | 0.933 | 0.883 | 0.983 | 0.940 | 0.872 | 1 |
| SLC1A2, IL1R2, OLAH, FAM20A, RETN | | | | | | | | | |
| RSAD2, IFI27, OAS1, IFI44L, ISG15, | 0.878 | 0.867 | 0.888 | 0.931 | 0.880 | 0.983 | 0.924 | 0.847 | 1 |
| SLC1A2, OLAH, FAM20A, MMP8 | | | | | | | | | |
| IFI27, OAS1, IFIT1, IFI44L, SLC1A2, | 0.878 | 0.868 | 0.888 | 0.933 | 0.883 | 0.983 | 0.926 | 0.849 | 1 |
| IL1R2, OLAH, FAM20A, MMP8 | | | | | | | | | |
| RSAD2, IFI27, OAS1, SIGLEC1, ISG15, | 0.878 | 0.869 | 0.887 | 0.923 | 0.868 | 0.978 | 0.946 | 0.889 | 1 |
| HERC6, IL1R2, OLAH, RETN | | | | | | | | | |
| IFIT1, IFI44L, SIGLEC1, ISG15, HERC6, | 0.878 | 0.868 | 0.887 | 0.923 | 0.869 | 0.977 | 0.963 | 0.909 | 1 |
| IL1R2, OLAH, FAM20A, MMP8 | | | | | | | | | |
| RSAD2, IFI27, OAS1, IFI44L, HERC6, | 0.878 | 0.867 | 0.888 | 0.933 | 0.883 | 0.982 | 0.921 | 0.839 | 1 |
| SLC1A2, IL1R2, OLAH, FAM20A | | | | | | | | | |
| IFI27, OAS1, IFI44L, HERC6, SLC1A2, | 0.878 | 0.867 | 0.889 | 0.936 | 0.887 | 0.985 | 0.923 | 0.842 | 1 |
| OLAH, FAM20A, RETN, MMP8 | | | | | | | | | |
| RSAD2, IFI27, IFIT1, IFI44L, SIGLEC1, | 0.878 | 0.868 | 0.888 | 0.931 | 0.878 | 0.984 | 0.941 | 0.876 | 1 |
| SLC1A2, OLAH, FAM20A, RETN | | | | | | | | | |
| RSAD2, OAS1, IFIT1, IFI44L, SIGLEC1, | 0.878 | 0.869 | 0.887 | 0.923 | 0.869 | 0.978 | 0.962 | 0.906 | 1 |
| HERC6, IL1R2, FAM20A, MMP8 | | | | | | | | | |
| IFI27, IFIT1, IFI44L, SIGLEC1, ISG15, | 0.878 | 0.868 | 0.887 | 0.930 | 0.879 | 0.981 | 0.941 | 0.869 | 1 |
| HERC6, SLC1A2, FAM20A, RETN | | | | | | | | | |
| IFI27, IFI44L, ISG15, HERC6, SLC1A2, | 0.878 | 0.868 | 0.888 | 0.931 | 0.879 | 0.983 | 0.924 | 0.844 | 1 |
| OLAH, FAM20A, RETN, MMP8 | | | | | | | | | |
| RSAD2, IFI27, OAS1, IFIT1, IFI44L, | 0.878 | 0.869 | 0.887 | 0.924 | 0.866 | 0.981 | 0.939 | 0.868 | 1 |
| SIGLEC1, ISG15, HERC6, FAM20A | | | | | | | | | |
| IFI27, OAS1, IFI44L, SIGLEC1, HERC6, | 0.878 | 0.868 | 0.888 | 0.936 | 0.891 | 0.981 | 0.942 | 0.872 | 1 |
| SLC1A2, IL1R2, FAM20A, RETN | | | | | | | | | |
| IFI27, OAS1, IFIT1, IFI44L, ISG15, | 0.878 | 0.867 | 0.888 | 0.922 | 0.862 | 0.981 | 0.942 | 0.877 | 1 |
| OLAH, FAM20A, RETN, MMP8 | | | | | | | | | |
| IFIT1, IFI44L, SIGLEC1, ISG15, HERC6, | 0.878 | 0.869 | 0.887 | 0.923 | 0.867 | 0.979 | 0.961 | 0.903 | 1 |
| IL1R2, FAM20A, RETN, MMP8 | | | | | | | | | |
| IFI27, ISG15, HERC6, SLC1A2, IL1R2, | 0.878 | 0.868 | 0.887 | 0.935 | 0.888 | 0.982 | 0.923 | 0.841 | 1 |
| OLAH, FAM20A, RETN, MMP8 | | | | | | | | | |
| IFI27, OAS1, IFIT1, IFI44L, SIGLEC1, | 0.878 | 0.869 | 0.887 | 0.928 | 0.877 | 0.978 | 0.954 | 0.907 | 1 |
| HERC6, IL1R2, RETN, MMP8 | | | | | | | | | |
| RSAD2, IFI27, OAS1, IFIT1, HERC6, | 0.878 | 0.868 | 0.887 | 0.924 | 0.868 | 0.980 | 0.945 | 0.885 | 1 |
| IL1R2, OLAH, RETN, MMP8 | | | | | | | | | |
| RSAD2, IFI27, OAS1, IFIT1, SIGLEC1, | 0.878 | 0.869 | 0.887 | 0.925 | 0.872 | 0.979 | 0.957 | 0.906 | 1 |
| HERC6, IL1R2, RETN, MMP8 | | | | | | | | | |
| RSAD2, IFI27, OAS1, IFIT1, IFI44L, | 0.878 | 0.868 | 0.887 | 0.924 | 0.867 | 0.981 | 0.929 | 0.859 | 1.000 |
| ISG15, IL1R2, OLAH, FAM20A | | | | | | | | | |
| IFI27, OAS1, IFIT1, ISG15, HERC6, | 0.878 | 0.868 | 0.888 | 0.933 | 0.884 | 0.983 | 0.915 | 0.828 | 1 |
| SLC1A2, OLAH, FAM20A, RETN | | | | | | | | | |
| RSAD2, IFI27, OAS1, IFIT1, SIGLEC1, | 0.878 | 0.868 | 0.887 | 0.931 | 0.878 | 0.984 | 0.942 | 0.876 | 1 |
| SLC1A2, OLAH, FAM20A, RETN | | | | | | | | | |
| IFI27, OAS1, IFIT1, IFI44L, SIGLEC1, | 0.878 | 0.868 | 0.887 | 0.923 | 0.866 | 0.980 | 0.937 | 0.865 | 1 |
| ISG15, HERC6, FAM20A, MMP8 | | | | | | | | | |
| OAS1, IFIT1, IFI44L, SIGLEC1, HERC6, | 0.878 | 0.868 | 0.887 | 0.924 | 0.869 | 0.979 | 0.963 | 0.908 | 1 |
| IL1R2, FAM20A, RETN, MMP8 | | | | | | | | | |
| RSAD2, IFI27, OAS1, IFIT1, ISG15, | 0.878 | 0.868 | 0.887 | 0.924 | 0.869 | 0.980 | 0.945 | 0.886 | 1 |
| HERC6, OLAH, RETN, MMP8 | | | | | | | | | |
| RSAD2, IFI27, IFI44L, ISG15, HERC6, | 0.878 | 0.867 | 0.888 | 0.931 | 0.880 | 0.983 | 0.934 | 0.854 | 1 |
| IL1R2, FAM20A, RETN, MMP8 | | | | | | | | | |
| RSAD2, IFI27, OAS1, IFI44L, SIGLEC1, | 0.878 | 0.869 | 0.886 | 0.927 | 0.879 | 0.975 | 0.949 | 0.900 | 0.997 |
| HERC6, IL1R2, OLAH, MMP8 | | | | | | | | | |
| IFI27, OAS1, IFI44L, SIGLEC1, ISG15, | 0.878 | 0.869 | 0.886 | 0.924 | 0.872 | 0.976 | 0.942 | 0.885 | 1.000 |
| HERC6, IL1R2, OLAH, RETN | | | | | | | | | |
| RSAD2, IFI27, SIGLEC1, ISG15, HERC6, | 0.877 | 0.868 | 0.887 | 0.925 | 0.871 | 0.979 | 0.953 | 0.903 | 1 |
| IL1R2, OLAH, RETN, MMP8 | | | | | | | | | |
| IFI27, OAS1, IFIT1, SIGLEC1, ISG15, | 0.877 | 0.869 | 0.886 | 0.925 | 0.872 | 0.978 | 0.957 | 0.907 | 1 |
| HERC6, IL1R2, RETN, MMP8 | | | | | | | | | |
| RSAD2, IFI27, OAS1, IFI44L, SLC1A2, | 0.877 | 0.867 | 0.888 | 0.934 | 0.884 | 0.983 | 0.926 | 0.849 | 1 |
| IL1R2, OLAH, FAM20A, MMP8 | | | | | | | | | |
| RSAD2, IFI27, OAS1, IFIT1, ISG15, | 0.877 | 0.868 | 0.887 | 0.928 | 0.877 | 0.979 | 0.929 | 0.848 | 1 |
| HERC6, IL1R2, FAM20A, MMP8 | | | | | | | | | |
| RSAD2, IFI27, OAS1, IFIT1, SIGLEC1, | 0.877 | 0.868 | 0.887 | 0.933 | 0.884 | 0.981 | 0.933 | 0.859 | 1 |
| HERC6, SLC1A2, FAM20A, RETN | | | | | | | | | |
| IFI27, IFIT1, HERC6, SLC1A2, IL1R2, | 0.877 | 0.867 | 0.887 | 0.932 | 0.882 | 0.982 | 0.929 | 0.853 | 1 |
| OLAH, FAM20A, RETN, MMP8 | | | | | | | | | |
| IFI27, OAS1, IFIT1, IFI44L, ISG15, | 0.877 | 0.867 | 0.888 | 0.926 | 0.869 | 0.983 | 0.934 | 0.855 | 1 |
| HERC6, FAM20A, RETN, MMP8 | | | | | | | | | |
| IFI27, OAS1, IFI44L, SIGLEC1, SLC1A2, | 0.877 | 0.867 | 0.887 | 0.930 | 0.877 | 0.982 | 0.943 | 0.879 | 1 |
| IL1R2, OLAH, FAM20A, RETN | | | | | | | | | |
| RSAD2, IFI27, OAS1, IFIT1, SIGLEC1, | 0.877 | 0.869 | 0.886 | 0.926 | 0.873 | 0.979 | 0.947 | 0.891 | 1 |
| ISG15, HERC6, OLAH, RETN | | | | | | | | | |
| IFI27, OAS1, IFIT1, ISG15, HERC6, | 0.877 | 0.868 | 0.887 | 0.924 | 0.868 | 0.979 | 0.945 | 0.885 | 1 |
| IL1R2, OLAH, RETN, MMP8 | | | | | | | | | |
| IFI27, OAS1, IFI44L, ISG15, HERC6, | 0.877 | 0.868 | 0.887 | 0.924 | 0.868 | 0.980 | 0.945 | 0.887 | 1 |
| IL1R2, OLAH, RETN, MMP8 | | | | | | | | | |
| RSAD2, IFI27, IFIT1, IFI44L, HERC6, | 0.877 | 0.867 | 0.887 | 0.933 | 0.883 | 0.982 | 0.923 | 0.843 | 1 |
| SLC1A2, IL1R2, OLAH, FAM20A | | | | | | | | | |
| RSAD2, IFI27, OAS1, IFI44L, SIGLEC1, | 0.877 | 0.869 | 0.886 | 0.924 | 0.870 | 0.979 | 0.942 | 0.883 | 1 |
| ISG15, HERC6, OLAH, RETN | | | | | | | | | |
| IFI27, SIGLEC1, ISG15, HERC6, | 0.877 | 0.867 | 0.888 | 0.933 | 0.884 | 0.982 | 0.938 | 0.870 | 1 |
| SLC1A2, IL1R2, OLAH, FAM20A, RETN | | | | | | | | | |
| RSAD2, IFI27, OAS1, IFIT1, HERC6, | 0.877 | 0.867 | 0.887 | 0.932 | 0.883 | 0.981 | 0.921 | 0.838 | 1 |
| SLC1A2, IL1R2, OLAH, FAM20A | | | | | | | | | |
| RSAD2, IFIT1, SIGLEC1, ISG15, | 0.877 | 0.868 | 0.887 | 0.922 | 0.867 | 0.976 | 0.962 | 0.906 | 1 |
| HERC6, IL1R2, OLAH, FAM20A, MMP8 | | | | | | | | | |
| IFI27, IFIT1, IFI44L, HERC6, SLC1A2, | 0.877 | 0.867 | 0.887 | 0.933 | 0.882 | 0.985 | 0.928 | 0.848 | 1 |
| OLAH, FAM20A, RETN, MMP8 | | | | | | | | | |
| IFI27, OAS1, IFIT1, IFI44L, ISG15, | 0.877 | 0.868 | 0.887 | 0.924 | 0.867 | 0.981 | 0.932 | 0.861 | 1 |
| IL1R2, OLAH, FAM20A, RETN | | | | | | | | | |
| IFI27, OAS1, IFI44L, SIGLEC1, ISG15, | 0.877 | 0.869 | 0.886 | 0.926 | 0.877 | 0.975 | 0.941 | 0.888 | 0.995 |
| HERC6, IL1R2, OLAH, MMP8 | | | | | | | | | |
| RSAD2, IFI27, OAS1, IFIT1, ISG15, | 0.877 | 0.867 | 0.888 | 0.923 | 0.863 | 0.982 | 0.943 | 0.880 | 1 |
| OLAH, FAM20A, RETN, MMP8 | | | | | | | | | |
| RSAD2, IFI27, OAS1, IFIT1, ISG15, | 0.877 | 0.867 | 0.887 | 0.930 | 0.880 | 0.981 | 0.920 | 0.837 | 1 |
| HERC6, SLC1A2, OLAH, FAM20A | | | | | | | | | |
| RSAD2, IFI27, OAS1, IFIT1, IFI44L, | 0.877 | 0.867 | 0.888 | 0.923 | 0.863 | 0.983 | 0.941 | 0.877 | 1 |
| OLAH, FAM20A, RETN, MMP8 | | | | | | | | | |
| IFI27, IFIT1, IFI44L, SIGLEC1, ISG15, | 0.877 | 0.869 | 0.886 | 0.926 | 0.874 | 0.979 | 0.946 | 0.893 | 0.998 |
| HERC6, IL1R2, OLAH, RETN | | | | | | | | | |
| RSAD2, IFI27, OAS1, IFIT1, IFI44L, | 0.877 | 0.868 | 0.887 | 0.924 | 0.867 | 0.981 | 0.945 | 0.887 | 1 |
| HERC6, OLAH, RETN, MMP8 | | | | | | | | | |
| RSAD2, IFI27, OAS1, IFIT1, ISG15, | 0.877 | 0.868 | 0.887 | 0.924 | 0.868 | 0.981 | 0.929 | 0.859 | 1.000 |
| IL1R2, OLAH, FAM20A, RETN | | | | | | | | | |
| IFI27, OAS1, IFIT1, SIGLEC1, HERC6, | 0.877 | 0.867 | 0.887 | 0.932 | 0.884 | 0.980 | 0.949 | 0.890 | 1 |
| SLC1A2, OLAH, RETN, MMP8 | | | | | | | | | |
| IFI27, IFIT1, SIGLEC1, ISG15, HERC6, | 0.877 | 0.868 | 0.886 | 0.926 | 0.872 | 0.980 | 0.954 | 0.906 | 1 |
| IL1R2, OLAH, RETN, MMP8 | | | | | | | | | |
| RSAD2, IFIT1, IFI44L, SIGLEC1, | 0.877 | 0.868 | 0.886 | 0.924 | 0.870 | 0.977 | 0.963 | 0.909 | 1 |
| HERC6, IL1R2, OLAH, FAM20A, MMP8 | | | | | | | | | |
| RSAD2, IFI27, OAS1, SIGLEC1, | 0.877 | 0.867 | 0.887 | 0.930 | 0.878 | 0.983 | 0.940 | 0.873 | 1 |
| SLC1A2, IL1R2, OLAH, FAM20A, RETN | | | | | | | | | |
| RSAD2, IFI27, OAS1, IFIT1, IFI44L, | 0.877 | 0.868 | 0.886 | 0.923 | 0.865 | 0.980 | 0.939 | 0.868 | 1 |
| SIGLEC1, HERC6, FAM20A, MMP8 | | | | | | | | | |
| IFI27, OAS1, IFIT1, IFI44L, SIGLEC1, | 0.877 | 0.869 | 0.886 | 0.926 | 0.877 | 0.975 | 0.947 | 0.896 | 0.997 |
| HERC6, IL1R2, OLAH, MMP8 | | | | | | | | | |
| RSAD2, IFI27, OAS1, IFI44L, SIGLEC1, | 0.877 | 0.867 | 0.887 | 0.930 | 0.878 | 0.982 | 0.943 | 0.878 | 1 |
| SLC1A2, IL1R2, OLAH, FAM20A | | | | | | | | | |
| OAS1, IFIT1, SIGLEC1, HERC6, IL1R2, | 0.877 | 0.868 | 0.886 | 0.925 | 0.870 | 0.980 | 0.962 | 0.907 | 1 |
| OLAH, FAM20A, RETN, MMP8 | | | | | | | | | |
| RSAD2, IFI27, IFIT1, IFI44L, SIGLEC1, | 0.877 | 0.868 | 0.886 | 0.926 | 0.874 | 0.979 | 0.946 | 0.893 | 0.998 |
| HERC6, IL1R2, OLAH, RETN | | | | | | | | | |
| IFI27, OAS1, IFIT1, IFI44L, SIGLEC1, | 0.877 | 0.869 | 0.886 | 0.927 | 0.877 | 0.977 | 0.941 | 0.886 | 0.996 |
| ISG15, HERC6, IL1R2, RETN | | | | | | | | | |
| IFI27, IFIT1, ISG15, SLC1A2, IL1R2, | 0.877 | 0.867 | 0.887 | 0.933 | 0.884 | 0.981 | 0.924 | 0.845 | 1 |
| OLAH, FAM20A, RETN, MMP8 | | | | | | | | | |
| IFI27, OAS1, IFIT1, IFI44L, HERC6, | 0.877 | 0.868 | 0.886 | 0.924 | 0.868 | 0.979 | 0.949 | 0.894 | 1 |
| IL1R2, OLAH, RETN, MMP8 | | | | | | | | | |
| IFI27, IFIT1, IFI44L, ISG15, SLC1A2, | 0.877 | 0.867 | 0.887 | 0.930 | 0.877 | 0.983 | 0.925 | 0.847 | 1 |
| OLAH, FAM20A, RETN, MMP8 | | | | | | | | | |
| RSAD2, IFI27, IFIT1, SIGLEC1, HERC6, | 0.877 | 0.868 | 0.887 | 0.935 | 0.889 | 0.981 | 0.943 | 0.873 | 1 |
| SLC1A2, IL1R2, FAM20A, RETN | | | | | | | | | |
| RSAD2, IFI27, IFIT1, SLC1A2, IL1R2, | 0.877 | 0.867 | 0.887 | 0.933 | 0.882 | 0.983 | 0.924 | 0.846 | 1 |
| OLAH, FAM20A, RETN, MMP8 | | | | | | | | | |
| IFI27, OAS1, IFIT1, IFI44L, ISG15, | 0.877 | 0.868 | 0.886 | 0.924 | 0.867 | 0.981 | 0.945 | 0.887 | 1 |
| HERC6, OLAH, RETN, MMP8 | | | | | | | | | |
| RSAD2, IFI27, OAS1, ISG15, HERC6, | 0.877 | 0.867 | 0.887 | 0.924 | 0.869 | 0.980 | 0.945 | 0.885 | 1 |
| IL1R2, OLAH, RETN, MMP8 | | | | | | | | | |
| RSAD2, IFI27, OAS1, IFIT1, IFI44L, | 0.877 | 0.869 | 0.885 | 0.924 | 0.871 | 0.977 | 0.953 | 0.902 | 1 |
| SIGLEC1, HERC6, IL1R2, RETN | | | | | | | | | |
| IFI27, IFIT1, SIGLEC1, ISG15, HERC6, | 0.877 | 0.867 | 0.887 | 0.935 | 0.889 | 0.981 | 0.947 | 0.875 | 1 |
| SLC1A2, IL1R2, FAM20A, RETN | | | | | | | | | |
| IFI27, OAS1, SIGLEC1, ISG15, HERC6, | 0.877 | 0.867 | 0.887 | 0.935 | 0.890 | 0.980 | 0.941 | 0.871 | 1 |
| SLC1A2, IL1R2, FAM20A, RETN | | | | | | | | | |
| IFI27, OAS1, IFIT1, ISG15, HERC6, | 0.877 | 0.867 | 0.887 | 0.933 | 0.885 | 0.981 | 0.916 | 0.832 | 1 |
| SLC1A2, IL1R2, OLAH, FAM20A | | | | | | | | | |
| RSAD2, IFI27, IFIT1, IFI44L, ISG15, | 0.877 | 0.867 | 0.887 | 0.928 | 0.877 | 0.979 | 0.932 | 0.850 | 1 |
| HERC6, IL1R2, FAM20A, MMP8 | | | | | | | | | |
| RSAD2, IFI27, IFIT1, IFI44L, ISG15, | 0.877 | 0.867 | 0.887 | 0.932 | 0.881 | 0.982 | 0.929 | 0.849 | 1 |
| HERC6, IL1R2, FAM20A, RETN | | | | | | | | | |
| RSAD2, IFI27, IFI44L, ISG15, HERC6, | 0.877 | 0.867 | 0.887 | 0.934 | 0.885 | 0.983 | 0.920 | 0.839 | 1 |
| SLC1A2, IL1R2, OLAH, FAM20A | | | | | | | | | |
| RSAD2, IFI27, OAS1, IFI44L, ISG15, | 0.877 | 0.867 | 0.887 | 0.924 | 0.867 | 0.981 | 0.928 | 0.856 | 1 |
| IL1R2, OLAH, FAM20A, RETN | | | | | | | | | |
| RSAD2, IFI27, IFIT1, IFI44L, ISG15, | 0.877 | 0.867 | 0.887 | 0.933 | 0.883 | 0.983 | 0.918 | 0.837 | 1.000 |
| HERC6, SLC1A2, OLAH, FAM20A | | | | | | | | | |
| IFI27, OAS1, IFIT1, IFI44L, SIGLEC1, | 0.877 | 0.868 | 0.885 | 0.927 | 0.879 | 0.976 | 0.942 | 0.889 | 0.996 |
| ISG15, HERC6, IL1R2, OLAH | | | | | | | | | |
| RSAD2, IFI27, OAS1, IFI44L, SIGLEC1, | 0.877 | 0.868 | 0.886 | 0.926 | 0.877 | 0.976 | 0.946 | 0.894 | 0.997 |
| ISG15, HERC6, IL1R2, OLAH | | | | | | | | | |
| IFI27, OAS1, IFIT1, SIGLEC1, ISG15, | 0.877 | 0.867 | 0.887 | 0.934 | 0.887 | 0.980 | 0.943 | 0.875 | 1 |
| SLC1A2, IL1R2, FAM20A, RETN | | | | | | | | | |
| RSAD2, IFI27, OAS1, IFIT1, IFI44L, | 0.877 | 0.868 | 0.885 | 0.926 | 0.877 | 0.975 | 0.948 | 0.897 | 0.998 |
| SIGLEC1, HERC6, IL1R2, OLAH | | | | | | | | | |
| IFI27, IFIT1, ISG15, HERC6, SLC1A2, | 0.877 | 0.867 | 0.887 | 0.931 | 0.880 | 0.982 | 0.921 | 0.838 | 1 |
| OLAH, FAM20A, RETN, MMP8 | | | | | | | | | |
| IFI27, OAS1, IFI44L, SIGLEC1, ISG15, | 0.877 | 0.868 | 0.885 | 0.928 | 0.877 | 0.979 | 0.952 | 0.902 | 1 |
| HERC6, IL1R2, RETN, MMP8 | | | | | | | | | |
| IFI27, IFI44L, HERC6, SLC1A2, IL1R2, | 0.877 | 0.867 | 0.887 | 0.932 | 0.882 | 0.982 | 0.932 | 0.856 | 1 |
| OLAH, FAM20A, RETN, MMP8 | | | | | | | | | |
| RSAD2, IFI27, IFI44L, SIGLEC1, | 0.877 | 0.867 | 0.887 | 0.933 | 0.883 | 0.983 | 0.941 | 0.874 | 1 |
| HERC6, SLC1A2, OLAH, FAM20A, RETN | | | | | | | | | |
| IFIT1, IFI44L, SIGLEC1, HERC6, IL1R2, | 0.877 | 0.867 | 0.886 | 0.925 | 0.870 | 0.980 | 0.961 | 0.904 | 1 |
| OLAH, FAM20A, RETN, MMP8 | | | | | | | | | |
| IFI27, IFIT1, SIGLEC1, ISG15, HERC6, | 0.877 | 0.867 | 0.887 | 0.930 | 0.877 | 0.983 | 0.951 | 0.879 | 1 |
| SLC1A2, FAM20A, RETN, MMP8 | | | | | | | | | |
| IFI27, IFIT1, IFI44L, SLC1A2, IL1R2, | 0.877 | 0.867 | 0.886 | 0.934 | 0.884 | 0.983 | 0.926 | 0.848 | 1 |
| OLAH, FAM20A, RETN, MMP8 | | | | | | | | | |
| RSAD2, IFI27, OAS1, IFIT1, SIGLEC1, | 0.877 | 0.868 | 0.886 | 0.920 | 0.862 | 0.978 | 0.945 | 0.887 | 1 |
| ISG15, HERC6, RETN, MMP8 | | | | | | | | | |
| RSAD2, IFI27, IFIT1, IFI44L, SIGLEC1, | 0.877 | 0.867 | 0.886 | 0.922 | 0.862 | 0.981 | 0.939 | 0.868 | 1 |
| ISG15, HERC6, FAM20A, MMP8 | | | | | | | | | |
| RSAD2, IFI27, OAS1, IFIT1, IFI44L, | 0.877 | 0.866 | 0.887 | 0.923 | 0.864 | 0.982 | 0.946 | 0.876 | 1 |
| SIGLEC1, FAM20A, RETN, MMP8 | | | | | | | | | |
| RSAD2, IFI27, IFI44L, SIGLEC1, ISG15, | 0.877 | 0.868 | 0.886 | 0.925 | 0.871 | 0.980 | 0.951 | 0.899 | 1 |
| HERC6, OLAH, RETN, MMP8 | | | | | | | | | |
| OAS1, IFIT1, SIGLEC1, ISG15, HERC6, | 0.877 | 0.868 | 0.886 | 0.923 | 0.867 | 0.979 | 0.961 | 0.903 | 1 |
| IL1R2, FAM20A, RETN, MMP8 | | | | | | | | | |
| IFI27, IFIT1, IFI44L, ISG15, HERC6, | 0.877 | 0.867 | 0.886 | 0.934 | 0.886 | 0.982 | 0.920 | 0.838 | 1 |
| SLC1A2, IL1R2, OLAH, FAM20A | | | | | | | | | |
| RSAD2, OAS1, IFIT1, SIGLEC1, ISG15, | 0.876 | 0.867 | 0.886 | 0.922 | 0.868 | 0.977 | 0.963 | 0.907 | 1 |
| HERC6, IL1R2, FAM20A, MMP8 | | | | | | | | | |
| RSAD2, IFI27, OAS1, IFI44L, HERC6, | 0.876 | 0.867 | 0.886 | 0.923 | 0.867 | 0.979 | 0.943 | 0.885 | 1 |
| IL1R2, OLAH, RETN, MMP8 | | | | | | | | | |
| IFI27, OAS1, SIGLEC1, ISG15, HERC6, | 0.876 | 0.866 | 0.887 | 0.930 | 0.877 | 0.984 | 0.943 | 0.872 | 1 |
| SLC1A2, FAM20A, RETN, MMP8 | | | | | | | | | |
| RSAD2, IFI27, OAS1, IFIT1, SIGLEC1, | 0.876 | 0.866 | 0.887 | 0.923 | 0.863 | 0.982 | 0.946 | 0.874 | 1 |
| ISG15, FAM20A, RETN, MMP8 | | | | | | | | | |
| IFI27, OAS1, SIGLEC1, HERC6, | 0.876 | 0.867 | 0.886 | 0.931 | 0.883 | 0.980 | 0.948 | 0.888 | 1 |
| SLC1A2, IL1R2, OLAH, RETN, MMP8 | | | | | | | | | |
| RSAD2, IFI27, OAS1, IFI44L, SIGLEC1, | 0.876 | 0.867 | 0.885 | 0.927 | 0.874 | 0.980 | 0.954 | 0.903 | 1 |
| HERC6, IL1R2, RETN, MMP8 | | | | | | | | | |
| RSAD2, IFI27, ISG15, HERC6, SLC1A2, | 0.876 | 0.867 | 0.886 | 0.931 | 0.881 | 0.981 | 0.916 | 0.832 | 1 |
| IL1R2, OLAH, FAM20A, RETN | | | | | | | | | |
| RSAD2, IFI27, IFIT1, IFI44L, SIGLEC1, | 0.876 | 0.866 | 0.887 | 0.923 | 0.864 | 0.982 | 0.946 | 0.874 | 1 |
| ISG15, FAM20A, RETN, MMP8 | | | | | | | | | |
| IFI27, OAS1, IFIT1, SLC1A2, IL1R2, | 0.876 | 0.866 | 0.886 | 0.933 | 0.883 | 0.983 | 0.923 | 0.844 | 1 |
| OLAH, FAM20A, RETN, MMP8 | | | | | | | | | |
| RSAD2, IFI27, OAS1, IFI44L, ISG15, | 0.876 | 0.865 | 0.887 | 0.923 | 0.864 | 0.983 | 0.938 | 0.871 | 1 |
| OLAH, FAM20A, RETN, MMP8 | | | | | | | | | |
| RSAD2, IFI27, IFI44L, SIGLEC1, ISG15, | 0.876 | 0.866 | 0.886 | 0.932 | 0.885 | 0.979 | 0.948 | 0.879 | 1 |
| HERC6, SLC1A2, IL1R2, FAM20A | | | | | | | | | |
| RSAD2, IFI27, OAS1, IFI44L, ISG15, | 0.876 | 0.866 | 0.887 | 0.931 | 0.880 | 0.982 | 0.927 | 0.844 | 1 |
| HERC6, IL1R2, FAM20A, RETN | | | | | | | | | |
| IFIT1, SIGLEC1, ISG15, HERC6, IL1R2, | 0.876 | 0.867 | 0.886 | 0.923 | 0.867 | 0.979 | 0.962 | 0.906 | 1 |
| OLAH, FAM20A, RETN, MMP8 | | | | | | | | | |
| RSAD2, IFI27, IFIT1, IFI44L, SIGLEC1, | 0.876 | 0.868 | 0.885 | 0.925 | 0.872 | 0.978 | 0.946 | 0.893 | 0.998 |
| ISG15, HERC6, OLAH, RETN | | | | | | | | | |
| RSAD2, IFI27, IFIT1, IFI44L, HERC6, | 0.876 | 0.867 | 0.886 | 0.924 | 0.868 | 0.979 | 0.948 | 0.894 | 1 |
| IL1R2, OLAH, RETN, MMP8 | | | | | | | | | |
| RSAD2, IFI27, IFI44L, SIGLEC1, ISG15, | 0.876 | 0.868 | 0.885 | 0.925 | 0.873 | 0.978 | 0.946 | 0.893 | 0.998 |
| HERC6, IL1R2, OLAH, RETN | | | | | | | | | |
| IFI27, IFI44L, SIGLEC1, HERC6, | 0.876 | 0.866 | 0.886 | 0.931 | 0.879 | 0.982 | 0.946 | 0.879 | 1 |
| SLC1A2, IL1R2, OLAH, FAM20A, RETN | | | | | | | | | |
| OAS1, IFIT1, IFI44L, SIGLEC1, ISG15, | 0.876 | 0.866 | 0.886 | 0.923 | 0.869 | 0.978 | 0.963 | 0.907 | 1 |
| HERC6, IL1R2, FAM20A, MMP8 | | | | | | | | | |
| RSAD2, IFI27, OAS1, ISG15, HERC6, | 0.876 | 0.866 | 0.886 | 0.934 | 0.886 | 0.982 | 0.917 | 0.831 | 1 |
| SLC1A2, OLAH, FAM20A, RETN | | | | | | | | | |
| RSAD2, IFI27, OAS1, SIGLEC1, HERC6, | 0.876 | 0.866 | 0.887 | 0.930 | 0.876 | 0.984 | 0.941 | 0.867 | 1 |
| SLC1A2, FAM20A, RETN, MMP8 | | | | | | | | | |
| IFI27, OAS1, IFIT1, HERC6, SLC1A2, | 0.876 | 0.866 | 0.886 | 0.932 | 0.882 | 0.981 | 0.915 | 0.828 | 1 |
| IL1R2, OLAH, FAM20A, RETN | | | | | | | | | |
| RSAD2, IFI27, OAS1, IFI44L, ISG15, | 0.876 | 0.867 | 0.886 | 0.924 | 0.868 | 0.980 | 0.942 | 0.883 | 1 |
| HERC6, OLAH, RETN, MMP8 | | | | | | | | | |
| IFI27, OAS1, IFI44L, SIGLEC1, ISG15, | 0.876 | 0.866 | 0.886 | 0.933 | 0.886 | 0.981 | 0.941 | 0.871 | 1 |
| SLC1A2, IL1R2, FAM20A, RETN | | | | | | | | | |
| RSAD2, IFI27, OAS1, HERC6, SLC1A2, | 0.876 | 0.866 | 0.886 | 0.933 | 0.885 | 0.982 | 0.917 | 0.831 | 1 |
| IL1R2, OLAH, FAM20A, RETN | | | | | | | | | |
| RSAD2, IFI27, OAS1, IFIT1, ISG15, | 0.876 | 0.867 | 0.885 | 0.930 | 0.879 | 0.981 | 0.928 | 0.846 | 1 |
| HERC6, IL1R2, FAM20A, RETN | | | | | | | | | |
| RSAD2, IFI27, IFI44L, SIGLEC1, ISG15, | 0.876 | 0.868 | 0.885 | 0.927 | 0.877 | 0.976 | 0.941 | 0.888 | 0.995 |
| HERC6, IL1R2, OLAH, MMP8 | | | | | | | | | |
| IFI27, OAS1, IFIT1, IFI44L, ISG15, | 0.876 | 0.866 | 0.886 | 0.929 | 0.876 | 0.982 | 0.935 | 0.858 | 1 |
| IL1R2, FAM20A, RETN, MMP8 | | | | | | | | | |
| RSAD2, IFI27, OAS1, IFIT1, ISG15, | 0.876 | 0.867 | 0.885 | 0.923 | 0.867 | 0.978 | 0.941 | 0.882 | 1 |
| HERC6, IL1R2, OLAH, RETN | | | | | | | | | |
| RSAD2, IFI27, OAS1, IFI44L, SIGLEC1, | 0.876 | 0.868 | 0.884 | 0.924 | 0.872 | 0.977 | 0.954 | 0.904 | 1 |
| ISG15, HERC6, IL1R2, RETN | | | | | | | | | |
| RSAD2, IFI27, IFIT1, IFI44L, ISG15, | 0.876 | 0.865 | 0.887 | 0.922 | 0.861 | 0.982 | 0.938 | 0.872 | 1 |
| OLAH, FAM20A, RETN, MMP8 | | | | | | | | | |
| RSAD2, IFI27, OAS1, IFI44L, ISG15, | 0.876 | 0.867 | 0.885 | 0.922 | 0.866 | 0.978 | 0.940 | 0.880 | 1 |
| HERC6, IL1R2, OLAH, RETN | | | | | | | | | |
| IFI27, IFIT1, IFI44L, ISG15, HERC6, | 0.876 | 0.866 | 0.886 | 0.932 | 0.882 | 0.982 | 0.918 | 0.836 | 1 |
| SLC1A2, OLAH, FAM20A, RETN | | | | | | | | | |
| RSAD2, IFI27, IFIT1, SIGLEC1, ISG15, | 0.876 | 0.867 | 0.885 | 0.926 | 0.873 | 0.978 | 0.942 | 0.887 | 0.998 |
| HERC6, IL1R2, OLAH, RETN | | | | | | | | | |
| IFI27, OAS1, IFIT1, IFI44L, SIGLEC1, | 0.876 | 0.867 | 0.885 | 0.927 | 0.876 | 0.978 | 0.942 | 0.888 | 0.997 |
| ISG15, HERC6, OLAH, MMP8 | | | | | | | | | |
| IFI27, OAS1, IFI44L, SIGLEC1, HERC6, | 0.876 | 0.866 | 0.886 | 0.931 | 0.879 | 0.984 | 0.942 | 0.869 | 1 |
| SLC1A2, FAM20A, RETN, MMP8 | | | | | | | | | |
| RSAD2, IFI27, IFIT1, ISG15, HERC6, | 0.876 | 0.866 | 0.886 | 0.932 | 0.883 | 0.982 | 0.915 | 0.831 | 0.999 |
| SLC1A2, IL1R2, OLAH, FAM20A | | | | | | | | | |
| RSAD2, IFI27, IFIT1, IFI44L, SIGLEC1, | 0.876 | 0.867 | 0.884 | 0.927 | 0.878 | 0.975 | 0.943 | 0.892 | 0.995 |
| HERC6, IL1R2, OLAH, MMP8 | | | | | | | | | |
| RSAD2, IFI27, OAS1, IFIT1, IFI44L, | 0.876 | 0.866 | 0.886 | 0.925 | 0.868 | 0.982 | 0.932 | 0.860 | 1 |
| IL1R2, OLAH, FAM20A, RETN | | | | | | | | | |
| RSAD2, IFI27, OAS1, IFIT1, SIGLEC1, | 0.876 | 0.867 | 0.885 | 0.935 | 0.889 | 0.981 | 0.943 | 0.875 | 1 |
| ISG15, SLC1A2, IL1R2, FAM20A | | | | | | | | | |
| RSAD2, IFI27, IFIT1, IFI44L, SIGLEC1, | 0.876 | 0.866 | 0.885 | 0.931 | 0.882 | 0.981 | 0.939 | 0.866 | 1 |
| HERC6, SLC1A2, FAM20A, RETN | | | | | | | | | |
| RSAD2, IFI27, OAS1, IFIT1, IFI44L, | 0.876 | 0.867 | 0.885 | 0.920 | 0.862 | 0.978 | 0.941 | 0.879 | 1 |
| SIGLEC1, HERC6, RETN, MMP8 | | | | | | | | | |
| IFI27, OAS1, ISG15, HERC6, SLC1A2, | 0.876 | 0.866 | 0.886 | 0.933 | 0.884 | 0.982 | 0.915 | 0.829 | 1 |
| IL1R2, OLAH, FAM20A, RETN | | | | | | | | | |
| RSAD2, IFI27, IFI44L, ISG15, HERC6, | 0.876 | 0.866 | 0.886 | 0.935 | 0.886 | 0.983 | 0.917 | 0.836 | 0.999 |
| SLC1A2, OLAH, FAM20A, RETN | | | | | | | | | |
| RSAD2, IFI27, OAS1, SIGLEC1, HERC6, | 0.876 | 0.866 | 0.886 | 0.934 | 0.888 | 0.980 | 0.940 | 0.868 | 1 |
| SLC1A2, IL1R2, FAM20A, RETN | | | | | | | | | |
| IFI27, IFI44L, ISG15, HERC6, SLC1A2, | 0.876 | 0.866 | 0.886 | 0.931 | 0.881 | 0.982 | 0.917 | 0.834 | 1 |
| IL1R2, OLAH, FAM20A, RETN | | | | | | | | | |
| RSAD2, IFI27, ISG15, SLC1A2, IL1R2, | 0.876 | 0.866 | 0.886 | 0.935 | 0.887 | 0.984 | 0.922 | 0.841 | 1 |
| OLAH, FAM20A, RETN, MMP8 | | | | | | | | | |
| IFI27, OAS1, SIGLEC1, ISG15, HERC6, | 0.876 | 0.866 | 0.885 | 0.932 | 0.884 | 0.981 | 0.947 | 0.886 | 1 |
| SLC1A2, OLAH, RETN, MMP8 | | | | | | | | | |
| RSAD2, IFI27, OAS1, IFIT1, SIGLEC1, | 0.876 | 0.866 | 0.885 | 0.923 | 0.866 | 0.980 | 0.939 | 0.868 | 1 |
| ISG15, HERC6, FAM20A, MMP8 | | | | | | | | | |
| RSAD2, IFI27, OAS1, IFIT1, SIGLEC1, | 0.876 | 0.867 | 0.884 | 0.925 | 0.873 | 0.977 | 0.950 | 0.896 | 1 |
| ISG15, HERC6, IL1R2, RETN | | | | | | | | | |
| IFI27, IFI44L, SIGLEC1, ISG15, HERC6, | 0.876 | 0.866 | 0.886 | 0.933 | 0.886 | 0.980 | 0.947 | 0.875 | 1 |
| SLC1A2, IL1R2, FAM20A, RETN | | | | | | | | | |
| RSAD2, IFI27, OAS1, IFI44L, SIGLEC1, | 0.876 | 0.865 | 0.886 | 0.935 | 0.890 | 0.979 | 0.943 | 0.872 | 1 |
| HERC6, SLC1A2, IL1R2, FAM20A | | | | | | | | | |
| RSAD2, IFI27, IFIT1, IFI44L, ISG15, | 0.876 | 0.866 | 0.885 | 0.924 | 0.867 | 0.981 | 0.932 | 0.861 | 1 |
| IL1R2, OLAH, FAM20A, RETN | | | | | | | | | |
| RSAD2, IFI27, OAS1, IFIT1, IFI44L, | 0.876 | 0.867 | 0.884 | 0.926 | 0.875 | 0.977 | 0.945 | 0.892 | 0.997 |
| SIGLEC1, HERC6, OLAH, MMP8 | | | | | | | | | |
| RSAD2, IFI27, OAS1, IFIT1, IFI44L, | 0.876 | 0.866 | 0.885 | 0.921 | 0.861 | 0.981 | 0.936 | 0.868 | 1 |
| ISG15, OLAH, FAM20A, RETN | | | | | | | | | |
| RSAD2, IFI27, IFIT1, IFI44L, SIGLEC1, | 0.875 | 0.867 | 0.884 | 0.934 | 0.889 | 0.980 | 0.945 | 0.875 | 1 |
| ISG15, SLC1A2, IL1R2, FAM20A | | | | | | | | | |
| RSAD2, IFI27, OAS1, SIGLEC1, ISG15, | 0.875 | 0.866 | 0.885 | 0.924 | 0.875 | 0.974 | 0.943 | 0.891 | 0.996 |
| HERC6, IL1R2, OLAH, MMP8 | | | | | | | | | |
| RSAD2, IFI27, OAS1, IFI44L, SIGLEC1, | 0.875 | 0.865 | 0.886 | 0.927 | 0.871 | 0.983 | 0.939 | 0.872 | 1 |
| SLC1A2, OLAH, FAM20A, RETN | | | | | | | | | |
| RSAD2, IFI27, IFI44L, SLC1A2, IL1R2, | 0.875 | 0.865 | 0.886 | 0.937 | 0.889 | 0.985 | 0.927 | 0.852 | 1 |
| OLAH, FAM20A, RETN, MMP8 | | | | | | | | | |
| RSAD2, IFI27, OAS1, IFIT1, IFI44L, | 0.875 | 0.866 | 0.885 | 0.933 | 0.885 | 0.981 | 0.945 | 0.878 | 1 |
| SIGLEC1, SLC1A2, IL1R2, FAM20A | | | | | | | | | |
| IFI27, OAS1, IFIT1, SIGLEC1, ISG15, | 0.875 | 0.867 | 0.884 | 0.924 | 0.875 | 0.974 | 0.943 | 0.891 | 0.996 |
| HERC6, IL1R2, OLAH, MMP8 | | | | | | | | | |
| IFI27, OAS1, IFIT1, SIGLEC1, HERC6, | 0.875 | 0.866 | 0.885 | 0.930 | 0.880 | 0.980 | 0.939 | 0.876 | 1 |
| SLC1A2, IL1R2, OLAH, RETN | | | | | | | | | |
| RSAD2, IFI27, IFIT1, SIGLEC1, ISG15, | 0.875 | 0.866 | 0.885 | 0.934 | 0.888 | 0.981 | 0.945 | 0.875 | 1 |
| SLC1A2, IL1R2, FAM20A, RETN | | | | | | | | | |
| IFI27, IFIT1, IFI44L, SIGLEC1, ISG15, | 0.875 | 0.867 | 0.884 | 0.927 | 0.878 | 0.976 | 0.940 | 0.886 | 0.994 |
| HERC6, IL1R2, OLAH, MMP8 | | | | | | | | | |
| RSAD2, IFI27, OAS1, IFIT1, IFI44L, | 0.875 | 0.866 | 0.884 | 0.922 | 0.865 | 0.978 | 0.941 | 0.882 | 1 |
| ISG15, HERC6, OLAH, RETN | | | | | | | | | |
| IFI27, OAS1, IFIT1, ISG15, SLC1A2, | 0.875 | 0.866 | 0.885 | 0.929 | 0.877 | 0.980 | 0.922 | 0.842 | 1 |
| IL1R2, OLAH, FAM20A, RETN | | | | | | | | | |
| RSAD2, IFI27, OAS1, IFI44L, HERC6, | 0.875 | 0.865 | 0.885 | 0.932 | 0.882 | 0.982 | 0.918 | 0.835 | 1 |
| SLC1A2, OLAH, FAM20A, RETN | | | | | | | | | |
| RSAD2, IFI27, OAS1, IFI44L, ISG15, | 0.875 | 0.864 | 0.886 | 0.927 | 0.870 | 0.984 | 0.938 | 0.861 | 1 |
| HERC6, FAM20A, RETN, MMP8 | | | | | | | | | |
| RSAD2, IFI27, OAS1, IFIT1, SIGLEC1, | 0.875 | 0.866 | 0.884 | 0.924 | 0.874 | 0.974 | 0.943 | 0.891 | 0.996 |
| ISG15, HERC6, IL1R2, OLAH | | | | | | | | | |
| RSAD2, IFI27, OAS1, IFIT1, IFI44L, | 0.875 | 0.865 | 0.885 | 0.930 | 0.877 | 0.983 | 0.920 | 0.839 | 1 |
| ISG15, SLC1A2, OLAH, FAM20A | | | | | | | | | |
| RSAD2, IFI27, IFIT1, IFI44L, HERC6, | 0.875 | 0.865 | 0.885 | 0.933 | 0.883 | 0.982 | 0.921 | 0.839 | 1 |
| SLC1A2, OLAH, FAM20A, RETN | | | | | | | | | |
| RSAD2, OAS1, IFIT1, SIGLEC1, HERC6, | 0.875 | 0.866 | 0.884 | 0.923 | 0.867 | 0.979 | 0.962 | 0.905 | 1 |
| IL1R2, FAM20A, RETN, MMP8 | | | | | | | | | |
| RSAD2, IFIT1, IFI44L, SIGLEC1, ISG15, | 0.875 | 0.866 | 0.884 | 0.922 | 0.867 | 0.977 | 0.963 | 0.907 | 1 |
| HERC6, IL1R2, FAM20A, MMP8 | | | | | | | | | |
| IFI27, OAS1, IFIT1, IFI44L, SIGLEC1, | 0.875 | 0.866 | 0.884 | 0.927 | 0.876 | 0.977 | 0.935 | 0.859 | 1 |
| HERC6, SLC1A2, FAM20A, MMP8 | | | | | | | | | |
| IFI27, OAS1, ISG15, SLC1A2, IL1R2, | 0.875 | 0.865 | 0.885 | 0.933 | 0.884 | 0.981 | 0.923 | 0.843 | 1 |
| OLAH, FAM20A, RETN, MMP8 | | | | | | | | | |
| IFI27, OAS1, IFI44L, SIGLEC1, HERC6, | 0.875 | 0.865 | 0.885 | 0.931 | 0.883 | 0.980 | 0.947 | 0.887 | 1 |
| SLC1A2, OLAH, RETN, MMP8 | | | | | | | | | |
| RSAD2, IFI27, OAS1, SLC1A2, IL1R2, | 0.875 | 0.865 | 0.885 | 0.933 | 0.883 | 0.982 | 0.923 | 0.842 | 1 |
| OLAH, FAM20A, RETN, MMP8 | | | | | | | | | |
| IFI27, IFIT1, IFI44L, ISG15, SLC1A2, | 0.875 | 0.866 | 0.884 | 0.931 | 0.880 | 0.982 | 0.917 | 0.836 | 0.999 |
| IL1R2, OLAH, FAM20A, RETN | | | | | | | | | |
| RSAD2, IFI27, IFI44L, ISG15, HERC6, | 0.875 | 0.866 | 0.884 | 0.924 | 0.869 | 0.980 | 0.945 | 0.889 | 1 |
| IL1R2, OLAH, RETN, MMP8 | | | | | | | | | |
| RSAD2, IFI27, IFI44L, SIGLEC1, | 0.875 | 0.864 | 0.886 | 0.930 | 0.877 | 0.983 | 0.946 | 0.881 | 1 |
| SLC1A2, IL1R2, OLAH, FAM20A, RETN | | | | | | | | | |
| RSAD2, IFI27, OAS1, IFI44L, ISG15, | 0.875 | 0.865 | 0.885 | 0.931 | 0.880 | 0.982 | 0.920 | 0.837 | 1 |
| SLC1A2, IL1R2, OLAH, FAM20A | | | | | | | | | |
| IFI27, OAS1, IFI44L, HERC6, SLC1A2, | 0.875 | 0.865 | 0.885 | 0.934 | 0.885 | 0.982 | 0.918 | 0.835 | 1 |
| IL1R2, OLAH, FAM20A, RETN | | | | | | | | | |
| IFI27, OAS1, IFIT1, IFI44L, SIGLEC1, | 0.875 | 0.866 | 0.884 | 0.924 | 0.870 | 0.978 | 0.958 | 0.904 | 1 |
| IL1R2, OLAH, RETN, MMP8 | | | | | | | | | |
| RSAD2, IFI27, OAS1, IFIT1, HERC6, | 0.875 | 0.865 | 0.885 | 0.933 | 0.883 | 0.982 | 0.917 | 0.831 | 1 |
| SLC1A2, OLAH, FAM20A, RETN | | | | | | | | | |
| RSAD2, OAS1, IFIT1, IFI44L, SIGLEC1, | 0.875 | 0.865 | 0.884 | 0.921 | 0.865 | 0.978 | 0.965 | 0.913 | 1 |
| IL1R2, OLAH, FAM20A, MMP8 | | | | | | | | | |
| RSAD2, IFI27, OAS1, SIGLEC1, HERC6, | 0.875 | 0.865 | 0.885 | 0.930 | 0.880 | 0.980 | 0.948 | 0.888 | 1 |
| SLC1A2, OLAH, RETN, MMP8 | | | | | | | | | |
| RSAD2, IFI27, OAS1, IFI44L, SIGLEC1, | 0.875 | 0.865 | 0.885 | 0.934 | 0.888 | 0.980 | 0.941 | 0.869 | 1 |
| ISG15, SLC1A2, IL1R2, FAM20A | | | | | | | | | |
| RSAD2, OAS1, IFIT1, SIGLEC1, ISG15, | 0.875 | 0.866 | 0.884 | 0.920 | 0.863 | 0.977 | 0.966 | 0.913 | 1 |
| IL1R2, OLAH, FAM20A, MMP8 | | | | | | | | | |
| RSAD2, IFI27, OAS1, IFI44L, SIGLEC1, | 0.875 | 0.866 | 0.884 | 0.925 | 0.873 | 0.977 | 0.943 | 0.890 | 0.997 |
| ISG15, HERC6, OLAH, MMP8 | | | | | | | | | |
| RSAD2, IFI27, OAS1, IFIT1, IFI44L, | 0.875 | 0.866 | 0.884 | 0.922 | 0.867 | 0.978 | 0.939 | 0.879 | 0.999 |
| HERC6, IL1R2, OLAH, RETN | | | | | | | | | |
| RSAD2, IFI27, OAS1, SIGLEC1, ISG15, | 0.875 | 0.865 | 0.884 | 0.925 | 0.872 | 0.978 | 0.954 | 0.902 | 1 |
| HERC6, IL1R2, RETN, MMP8 | | | | | | | | | |
| RSAD2, IFI27, OAS1, IFIT1, SLC1A2, | 0.875 | 0.865 | 0.885 | 0.926 | 0.870 | 0.981 | 0.928 | 0.854 | 1 |
| OLAH, FAM20A, RETN, MMP8 | | | | | | | | | |
| RSAD2, IFI27, IFIT1, SIGLEC1, ISG15, | 0.875 | 0.865 | 0.884 | 0.930 | 0.879 | 0.981 | 0.938 | 0.865 | 1 |
| HERC6, SLC1A2, FAM20A, RETN | | | | | | | | | |
| IFIT1, IFI44L, SIGLEC1, ISG15, HERC6, | 0.875 | 0.865 | 0.885 | 0.930 | 0.881 | 0.978 | 0.941 | 0.871 | 1 |
| SLC1A2, OLAH, FAM20A, MMP8 | | | | | | | | | |
| RSAD2, IFI27, OAS1, IFI44L, SIGLEC1, | 0.875 | 0.865 | 0.884 | 0.921 | 0.863 | 0.979 | 0.945 | 0.881 | 1 |
| ISG15, HERC6, RETN, MMP8 | | | | | | | | | |
| RSAD2, IFI27, IFIT1, IFI44L, SIGLEC1, | 0.875 | 0.866 | 0.883 | 0.926 | 0.876 | 0.976 | 0.941 | 0.887 | 0.995 |
| ISG15, HERC6, IL1R2, OLAH | | | | | | | | | |
| IFI27, OAS1, IFIT1, SIGLEC1, ISG15, | 0.875 | 0.865 | 0.885 | 0.930 | 0.881 | 0.978 | 0.940 | 0.878 | 1 |
| HERC6, SLC1A2, OLAH, RETN | | | | | | | | | |
| IFI27, OAS1, SIGLEC1, ISG15, HERC6, | 0.875 | 0.865 | 0.884 | 0.927 | 0.877 | 0.978 | 0.939 | 0.876 | 1 |
| SLC1A2, IL1R2, OLAH, RETN | | | | | | | | | |
| RSAD2, IFI27, OAS1, IFIT1, ISG15, | 0.875 | 0.865 | 0.884 | 0.930 | 0.879 | 0.980 | 0.921 | 0.838 | 1 |
| SLC1A2, IL1R2, OLAH, FAM20A | | | | | | | | | |
| IFI27, OAS1, IFIT1, ISG15, SLC1A2, | 0.875 | 0.864 | 0.885 | 0.929 | 0.876 | 0.982 | 0.926 | 0.850 | 1 |
| OLAH, FAM20A, RETN, MMP8 | | | | | | | | | |
| RSAD2, IFI27, OAS1, IFIT1, SIGLEC1, | 0.875 | 0.866 | 0.884 | 0.924 | 0.875 | 0.974 | 0.942 | 0.890 | 0.995 |
| HERC6, IL1R2, OLAH, MMP8 | | | | | | | | | |
| IFI27, OAS1, IFIT1, IFI44L, SLC1A2, | 0.875 | 0.864 | 0.885 | 0.929 | 0.873 | 0.984 | 0.929 | 0.854 | 1 |
| OLAH, FAM20A, RETN, MMP8 | | | | | | | | | |
| RSAD2, IFI27, OAS1, IFIT1, SIGLEC1, | 0.875 | 0.865 | 0.884 | 0.934 | 0.886 | 0.981 | 0.943 | 0.877 | 1 |
| SLC1A2, IL1R2, FAM20A, RETN | | | | | | | | | |
| RSAD2, IFI27, IFIT1, ISG15, SLC1A2, | 0.875 | 0.865 | 0.884 | 0.928 | 0.877 | 0.980 | 0.918 | 0.837 | 1.000 |
| IL1R2, OLAH, FAM20A, RETN | | | | | | | | | |
| RSAD2, IFI27, OAS1, IFIT1, IFI44L, | 0.875 | 0.866 | 0.883 | 0.926 | 0.874 | 0.978 | 0.943 | 0.890 | 0.997 |
| SIGLEC1, ISG15, HERC6, OLAH | | | | | | | | | |
| IFI27, OAS1, IFIT1, HERC6, SLC1A2, | 0.875 | 0.865 | 0.884 | 0.929 | 0.879 | 0.979 | 0.939 | 0.874 | 1 |
| IL1R2, OLAH, RETN, MMP8 | | | | | | | | | |
| IFI27, OAS1, IFIT1, HERC6, SLC1A2, | 0.875 | 0.865 | 0.885 | 0.932 | 0.883 | 0.981 | 0.925 | 0.842 | 1 |
| IL1R2, FAM20A, RETN, MMP8 | | | | | | | | | |
| RSAD2, IFI27, IFIT1, IFI44L, ISG15, | 0.875 | 0.865 | 0.884 | 0.931 | 0.880 | 0.982 | 0.917 | 0.836 | 0.999 |
| SLC1A2, IL1R2, OLAH, FAM20A | | | | | | | | | |
| RSAD2, IFI27, IFIT1, ISG15, HERC6, | 0.875 | 0.865 | 0.885 | 0.930 | 0.879 | 0.982 | 0.915 | 0.830 | 1 |
| SLC1A2, OLAH, FAM20A, RETN | | | | | | | | | |
| IFI27, OAS1, IFIT1, IFI44L, ISG15, | 0.875 | 0.865 | 0.884 | 0.930 | 0.877 | 0.982 | 0.921 | 0.840 | 1 |
| SLC1A2, IL1R2, OLAH, FAM20A | | | | | | | | | |
| IFI27, OAS1, IFIT1, IFI44L, ISG15, | 0.875 | 0.866 | 0.884 | 0.923 | 0.869 | 0.977 | 0.937 | 0.875 | 0.998 |
| HERC6, IL1R2, OLAH, RETN | | | | | | | | | |
| IFI27, OAS1, IFI44L, ISG15, SLC1A2, | 0.875 | 0.863 | 0.886 | 0.931 | 0.878 | 0.983 | 0.928 | 0.852 | 1 |
| OLAH, FAM20A, RETN, MMP8 | | | | | | | | | |
| RSAD2, IFI27, IFIT1, IFI44L, ISG15, | 0.875 | 0.865 | 0.884 | 0.923 | 0.866 | 0.979 | 0.947 | 0.893 | 1 |
| HERC6, OLAH, RETN, MMP8 | | | | | | | | | |
| RSAD2, IFI27, IFIT1, ISG15, HERC6, | 0.875 | 0.865 | 0.884 | 0.924 | 0.869 | 0.980 | 0.946 | 0.891 | 1 |
| IL1R2, OLAH, RETN, MMP8 | | | | | | | | | |
| RSAD2, IFI27, OAS1, IFIT1, SIGLEC1, | 0.875 | 0.865 | 0.884 | 0.932 | 0.883 | 0.981 | 0.946 | 0.885 | 1 |
| HERC6, SLC1A2, OLAH, RETN | | | | | | | | | |
| IFI27, IFIT1, IFI44L, HERC6, SLC1A2, | 0.875 | 0.865 | 0.884 | 0.931 | 0.879 | 0.983 | 0.925 | 0.845 | 1 |
| IL1R2, OLAH, FAM20A, RETN | | | | | | | | | |
| RSAD2, IFI27, SIGLEC1, ISG15, HERC6, | 0.874 | 0.864 | 0.885 | 0.929 | 0.876 | 0.983 | 0.945 | 0.871 | 1 |
| SLC1A2, FAM20A, RETN, MMP8 | | | | | | | | | |
| RSAD2, IFIT1, SIGLEC1, ISG15, | 0.874 | 0.865 | 0.883 | 0.923 | 0.867 | 0.979 | 0.961 | 0.901 | 1 |
| HERC6, IL1R2, FAM20A, RETN, MMP8 | | | | | | | | | |
| OAS1, IFIT1, SIGLEC1, HERC6, | 0.874 | 0.865 | 0.884 | 0.929 | 0.882 | 0.975 | 0.961 | 0.903 | 1 |
| SLC1A2, IL1R2, OLAH, FAM20A, MMP8 | | | | | | | | | |
| IFI27, OAS1, IFIT1, IFI44L, SIGLEC1, | 0.874 | 0.865 | 0.884 | 0.931 | 0.884 | 0.979 | 0.938 | 0.875 | 1 |
| HERC6, SLC1A2, OLAH, RETN | | | | | | | | | |
| RSAD2, IFI27, IFIT1, HERC6, SLC1A2, | 0.874 | 0.865 | 0.884 | 0.931 | 0.881 | 0.981 | 0.918 | 0.834 | 1 |
| IL1R2, OLAH, FAM20A, RETN | | | | | | | | | |
| RSAD2, IFI27, IFIT1, ISG15, SLC1A2, | 0.874 | 0.864 | 0.885 | 0.929 | 0.875 | 0.982 | 0.926 | 0.850 | 1 |
| OLAH, FAM20A, RETN, MMP8 | | | | | | | | | |
| IFI27, OAS1, IFIT1, IFI44L, ISG15, | 0.874 | 0.865 | 0.884 | 0.929 | 0.875 | 0.983 | 0.917 | 0.835 | 1.000 |
| SLC1A2, OLAH, FAM20A, RETN | | | | | | | | | |
| RSAD2, IFI27, OAS1, IFIT1, ISG15, | 0.874 | 0.864 | 0.885 | 0.925 | 0.868 | 0.982 | 0.935 | 0.857 | 1 |
| HERC6, FAM20A, RETN, MMP8 | | | | | | | | | |
| RSAD2, IFIT1, SIGLEC1, HERC6, | 0.874 | 0.865 | 0.884 | 0.926 | 0.871 | 0.980 | 0.961 | 0.904 | 1 |
| IL1R2, OLAH, FAM20A, RETN, MMP8 | | | | | | | | | |
| IFIT1, IFI44L, SIGLEC1, ISG15, HERC6, | 0.874 | 0.864 | 0.884 | 0.922 | 0.864 | 0.980 | 0.952 | 0.895 | 1 |
| OLAH, FAM20A, RETN, MMP8 | | | | | | | | | |
| RSAD2, IFI27, IFIT1, IFI44L, ISG15, | 0.874 | 0.864 | 0.885 | 0.926 | 0.869 | 0.982 | 0.937 | 0.859 | 1 |
| HERC6, FAM20A, RETN, MMP8 | | | | | | | | | |
| IFI27, OAS1, IFIT1, IFI44L, SIGLEC1, | 0.874 | 0.864 | 0.884 | 0.927 | 0.872 | 0.982 | 0.942 | 0.869 | 1 |
| SLC1A2, FAM20A, RETN, MMP8 | | | | | | | | | |
| IFI27, OAS1, IFI44L, SIGLEC1, ISG15, | 0.874 | 0.864 | 0.884 | 0.927 | 0.874 | 0.980 | 0.939 | 0.865 | 1 |
| HERC6, SLC1A2, FAM20A, RETN | | | | | | | | | |
| RSAD2, IFI27, IFI44L, SIGLEC1, | 0.874 | 0.864 | 0.885 | 0.934 | 0.886 | 0.981 | 0.947 | 0.877 | 1 |
| HERC6, SLC1A2, IL1R2, FAM20A, RETN | | | | | | | | | |
| RSAD2, IFI27, IFI44L, ISG15, SLC1A2, | 0.874 | 0.864 | 0.884 | 0.931 | 0.880 | 0.982 | 0.918 | 0.837 | 1.000 |
| IL1R2, OLAH, FAM20A, RETN | | | | | | | | | |
| RSAD2, IFI27, OAS1, SIGLEC1, ISG15, | 0.874 | 0.864 | 0.884 | 0.933 | 0.885 | 0.980 | 0.943 | 0.873 | 1 |
| SLC1A2, IL1R2, FAM20A, RETN | | | | | | | | | |
| OAS1, IFIT1, IFI44L, SIGLEC1, ISG15, | 0.874 | 0.864 | 0.884 | 0.920 | 0.863 | 0.977 | 0.966 | 0.913 | 1 |
| IL1R2, OLAH, FAM20A, MMP8 | | | | | | | | | |
| RSAD2, IFI27, IFIT1, IFI44L, SLC1A2, | 0.874 | 0.864 | 0.884 | 0.928 | 0.874 | 0.983 | 0.929 | 0.854 | 1 |
| OLAH, FAM20A, RETN, MMP8 | | | | | | | | | |
| OAS1, IFIT1, IFI44L, SIGLEC1, HERC6, | 0.874 | 0.864 | 0.884 | 0.920 | 0.862 | 0.979 | 0.951 | 0.894 | 1 |
| OLAH, FAM20A, RETN, MMP8 | | | | | | | | | |
| IFI27, OAS1, IFI44L, ISG15, SLC1A2, | 0.874 | 0.864 | 0.884 | 0.931 | 0.880 | 0.983 | 0.916 | 0.833 | 1.000 |
| IL1R2, OLAH, FAM20A, RETN | | | | | | | | | |
| IFI27, OAS1, IFIT1, IFI44L, SIGLEC1, | 0.874 | 0.865 | 0.884 | 0.925 | 0.870 | 0.980 | 0.938 | 0.865 | 1 |
| ISG15, SLC1A2, FAM20A, RETN | | | | | | | | | |
| RSAD2, IFI27, IFIT1, IFI44L, SIGLEC1, | 0.874 | 0.865 | 0.883 | 0.924 | 0.872 | 0.977 | 0.953 | 0.908 | 0.999 |
| HERC6, IL1R2, RETN, MMP8 | | | | | | | | | |
| RSAD2, IFI27, OAS1, IFIT1, IFI44L, | 0.874 | 0.864 | 0.885 | 0.928 | 0.875 | 0.981 | 0.934 | 0.856 | 1 |
| IL1R2, FAM20A, RETN, MMP8 | | | | | | | | | |
| IFIT1, IFI44L, SIGLEC1, HERC6, | 0.874 | 0.864 | 0.884 | 0.929 | 0.882 | 0.976 | 0.960 | 0.897 | 1 |
| SLC1A2, IL1R2, OLAH, FAM20A, MMP8 | | | | | | | | | |
| RSAD2, IFI27, OAS1, IFI44L, SIGLEC1, | 0.874 | 0.865 | 0.883 | 0.931 | 0.882 | 0.980 | 0.943 | 0.881 | 1 |
| HERC6, SLC1A2, OLAH, RETN | | | | | | | | | |
| RSAD2, IFI27, SIGLEC1, HERC6, | 0.874 | 0.865 | 0.884 | 0.931 | 0.883 | 0.980 | 0.951 | 0.898 | 1 |
| SLC1A2, IL1R2, OLAH, RETN, MMP8 | | | | | | | | | |
| RSAD2, IFI27, OAS1, ISG15, SLC1A2, | 0.874 | 0.864 | 0.884 | 0.932 | 0.882 | 0.981 | 0.915 | 0.830 | 1 |
| IL1R2, OLAH, FAM20A, RETN | | | | | | | | | |
| IFI27, IFIT1, IFI44L, ISG15, HERC6, | 0.874 | 0.865 | 0.883 | 0.922 | 0.865 | 0.978 | 0.942 | 0.884 | 1 |
| IL1R2, OLAH, RETN, MMP8 | | | | | | | | | |
| OAS1, IFIT1, IFI44L, SIGLEC1, HERC6, | 0.874 | 0.864 | 0.884 | 0.927 | 0.881 | 0.973 | 0.961 | 0.901 | 1 |
| SLC1A2, IL1R2, FAM20A, MMP8 | | | | | | | | | |
| RSAD2, IFI27, IFI44L, ISG15, SLC1A2, | 0.874 | 0.863 | 0.885 | 0.931 | 0.879 | 0.984 | 0.925 | 0.849 | 1 |
| OLAH, FAM20A, RETN, MMP8 | | | | | | | | | |
| IFI27, OAS1, IFI44L, SLC1A2, IL1R2, | 0.874 | 0.863 | 0.885 | 0.936 | 0.888 | 0.985 | 0.927 | 0.851 | 1 |
| OLAH, FAM20A, RETN, MMP8 | | | | | | | | | |
| RSAD2, OAS1, IFI44L, SIGLEC1, | 0.874 | 0.864 | 0.884 | 0.921 | 0.865 | 0.976 | 0.961 | 0.903 | 1 |
| HERC6, IL1R2, OLAH, FAM20A, MMP8 | | | | | | | | | |
| RSAD2, IFI27, IFI44L, HERC6, SLC1A2, | 0.874 | 0.864 | 0.884 | 0.932 | 0.882 | 0.982 | 0.923 | 0.842 | 1 |
| IL1R2, OLAH, FAM20A, RETN | | | | | | | | | |
| IFI27, OAS1, IFIT1, IFI44L, HERC6, | 0.874 | 0.864 | 0.884 | 0.931 | 0.885 | 0.977 | 0.925 | 0.838 | 1 |
| SLC1A2, IL1R2, FAM20A, MMP8 | | | | | | | | | |
| IFI27, IFIT1, IFI44L, SIGLEC1, ISG15, | 0.874 | 0.865 | 0.883 | 0.925 | 0.871 | 0.979 | 0.953 | 0.900 | 1 |
| IL1R2, OLAH, RETN, MMP8 | | | | | | | | | |
| IFI27, OAS1, IFIT1, SIGLEC1, HERC6, | 0.874 | 0.865 | 0.883 | 0.928 | 0.881 | 0.975 | 0.958 | 0.907 | 1 |
| SLC1A2, IL1R2, RETN, MMP8 | | | | | | | | | |
| RSAD2, IFI27, OAS1, SIGLEC1, HERC6, | 0.874 | 0.864 | 0.884 | 0.929 | 0.878 | 0.980 | 0.945 | 0.883 | 1 |
| SLC1A2, IL1R2, OLAH, RETN | | | | | | | | | |
| RSAD2, IFI27, OAS1, IFI44L, ISG15, | 0.874 | 0.865 | 0.883 | 0.927 | 0.878 | 0.976 | 0.937 | 0.879 | 0.995 |
| HERC6, IL1R2, OLAH, MMP8 | | | | | | | | | |
| RSAD2, IFI27, OAS1, IFIT1, SIGLEC1, | 0.874 | 0.865 | 0.883 | 0.923 | 0.871 | 0.975 | 0.936 | 0.878 | 0.994 |
| ISG15, HERC6, OLAH, MMP8 | | | | | | | | | |
| RSAD2, IFI27, OAS1, IFIT1, IFI44L, | 0.874 | 0.864 | 0.884 | 0.926 | 0.873 | 0.978 | 0.934 | 0.856 | 1 |
| ISG15, IL1R2, FAM20A, MMP8 | | | | | | | | | |
| IFI27, OAS1, IFIT1, SIGLEC1, ISG15, | 0.874 | 0.864 | 0.883 | 0.923 | 0.868 | 0.978 | 0.960 | 0.911 | 1 |
| IL1R2, OLAH, RETN, MMP8 | | | | | | | | | |
| IFIT1, IFI44L, SIGLEC1, ISG15, | 0.874 | 0.865 | 0.883 | 0.927 | 0.880 | 0.975 | 0.961 | 0.903 | 1 |
| SLC1A2, IL1R2, OLAH, FAM20A, MMP8 | | | | | | | | | |
| RSAD2, IFI27, OAS1, IFIT1, IFI44L, | 0.874 | 0.864 | 0.883 | 0.930 | 0.876 | 0.983 | 0.920 | 0.837 | 1 |
| SLC1A2, IL1R2, OLAH, FAM20A | | | | | | | | | |
| OAS1, IFIT1, SIGLEC1, ISG15, HERC6, | 0.874 | 0.864 | 0.884 | 0.921 | 0.864 | 0.979 | 0.952 | 0.895 | 1 |
| OLAH, FAM20A, RETN, MMP8 | | | | | | | | | |
| RSAD2, IFI27, IFIT1, SIGLEC1, HERC6, | 0.874 | 0.864 | 0.883 | 0.931 | 0.882 | 0.980 | 0.951 | 0.899 | 1 |
| SLC1A2, OLAH, RETN, MMP8 | | | | | | | | | |
| IFI27, OAS1, IFIT1, SIGLEC1, ISG15, | 0.874 | 0.864 | 0.884 | 0.930 | 0.880 | 0.979 | 0.937 | 0.877 | 0.997 |
| HERC6, SLC1A2, OLAH, MMP8 | | | | | | | | | |
| IFI27, IFIT1, ISG15, HERC6, SLC1A2, | 0.874 | 0.864 | 0.884 | 0.931 | 0.881 | 0.981 | 0.917 | 0.834 | 1 |
| IL1R2, OLAH, FAM20A, RETN | | | | | | | | | |
| RSAD2, OAS1, SIGLEC1, ISG15, | 0.874 | 0.864 | 0.883 | 0.917 | 0.860 | 0.975 | 0.962 | 0.905 | 1 |
| HERC6, IL1R2, OLAH, FAM20A, MMP8 | | | | | | | | | |
| RSAD2, OAS1, IFIT1, IFI44L, SIGLEC1, | 0.874 | 0.864 | 0.883 | 0.918 | 0.860 | 0.975 | 0.953 | 0.898 | 1 |
| HERC6, IL1R2, OLAH, FAM20A | | | | | | | | | |
| IFI27, OAS1, IFIT1, SIGLEC1, ISG15, | 0.874 | 0.864 | 0.884 | 0.926 | 0.870 | 0.982 | 0.943 | 0.868 | 1 |
| SLC1A2, FAM20A, RETN, MMP8 | | | | | | | | | |
| RSAD2, IFI27, OAS1, IFIT1, IFI44L, | 0.874 | 0.864 | 0.883 | 0.921 | 0.863 | 0.978 | 0.957 | 0.901 | 1 |
| SIGLEC1, OLAH, RETN, MMP8 | | | | | | | | | |
| RSAD2, IFI27, OAS1, IFI44L, SIGLEC1, | 0.874 | 0.863 | 0.885 | 0.924 | 0.866 | 0.983 | 0.947 | 0.875 | 1 |
| ISG15, FAM20A, RETN, MMP8 | | | | | | | | | |
| RSAD2, IFI27, IFI44L, SIGLEC1, ISG15, | 0.874 | 0.864 | 0.883 | 0.933 | 0.885 | 0.980 | 0.943 | 0.872 | 1 |
| SLC1A2, IL1R2, FAM20A, RETN | | | | | | | | | |
| RSAD2, IFI27, IFIT1, IFI44L, ISG15, | 0.874 | 0.864 | 0.883 | 0.928 | 0.874 | 0.982 | 0.916 | 0.834 | 0.999 |
| SLC1A2, OLAH, FAM20A, RETN | | | | | | | | | |
| RSAD2, IFI27, IFIT1, IFI44L, SIGLEC1, | 0.874 | 0.863 | 0.884 | 0.934 | 0.886 | 0.981 | 0.943 | 0.874 | 1 |
| SLC1A2, IL1R2, FAM20A, RETN | | | | | | | | | |
| IFI27, IFIT1, IFI44L, SIGLEC1, ISG15, | 0.874 | 0.865 | 0.883 | 0.923 | 0.870 | 0.976 | 0.952 | 0.906 | 0.999 |
| HERC6, IL1R2, RETN, MMP8 | | | | | | | | | |
| IFI27, OAS1, IFIT1, SIGLEC1, ISG15, | 0.874 | 0.864 | 0.883 | 0.928 | 0.879 | 0.978 | 0.939 | 0.880 | 0.998 |
| HERC6, SLC1A2, IL1R2, OLAH | | | | | | | | | |
| OAS1, IFIT1, IFI44L, SIGLEC1, HERC6, | 0.874 | 0.863 | 0.884 | 0.927 | 0.878 | 0.977 | 0.938 | 0.869 | 1 |
| SLC1A2, OLAH, FAM20A, MMP8 | | | | | | | | | |
| IFI27, OAS1, SIGLEC1, ISG15, HERC6, | 0.874 | 0.864 | 0.883 | 0.929 | 0.880 | 0.978 | 0.940 | 0.882 | 0.998 |
| SLC1A2, IL1R2, OLAH, MMP8 | | | | | | | | | |
| RSAD2, IFI27, OAS1, IFIT1, IFI44L, | 0.874 | 0.864 | 0.883 | 0.925 | 0.875 | 0.975 | 0.941 | 0.887 | 0.996 |
| HERC6, IL1R2, OLAH, MMP8 | | | | | | | | | |
| IFIT1, SIGLEC1, ISG15, HERC6, | 0.873 | 0.864 | 0.883 | 0.928 | 0.882 | 0.975 | 0.960 | 0.896 | 1 |
| SLC1A2, IL1R2, OLAH, FAM20A, MMP8 | | | | | | | | | |
| RSAD2, IFI27, OAS1, IFIT1, ISG15, | 0.873 | 0.863 | 0.884 | 0.930 | 0.877 | 0.982 | 0.934 | 0.856 | 1 |
| IL1R2, FAM20A, RETN, MMP8 | | | | | | | | | |
| IFI27, OAS1, IFI44L, SIGLEC1, ISG15, | 0.873 | 0.864 | 0.883 | 0.926 | 0.873 | 0.980 | 0.953 | 0.901 | 1 |
| IL1R2, OLAH, RETN, MMP8 | | | | | | | | | |
| RSAD2, IFIT1, IFI44L, SIGLEC1, | 0.873 | 0.863 | 0.884 | 0.929 | 0.880 | 0.977 | 0.934 | 0.862 | 1 |
| HERC6, SLC1A2, OLAH, FAM20A, MMP8 | | | | | | | | | |
| IFI27, OAS1, IFIT1, IFI44L, SIGLEC1, | 0.873 | 0.865 | 0.882 | 0.925 | 0.875 | 0.975 | 0.941 | 0.888 | 0.994 |
| ISG15, IL1R2, OLAH, MMP8 | | | | | | | | | |
| IFI27, IFIT1, SIGLEC1, HERC6, | 0.873 | 0.864 | 0.883 | 0.930 | 0.881 | 0.980 | 0.957 | 0.908 | 1 |
| SLC1A2, IL1R2, OLAH, RETN, MMP8 | | | | | | | | | |
| IFI27, OAS1, ISG15, HERC6, SLC1A2, | 0.873 | 0.864 | 0.883 | 0.934 | 0.887 | 0.982 | 0.925 | 0.838 | 1 |
| IL1R2, FAM20A, RETN, MMP8 | | | | | | | | | |
| RSAD2, IFI27, IFIT1, SIGLEC1, ISG15, | 0.873 | 0.864 | 0.882 | 0.925 | 0.876 | 0.974 | 0.942 | 0.890 | 0.995 |
| HERC6, IL1R2, OLAH, MMP8 | | | | | | | | | |
| RSAD2, IFI27, OAS1, IFIT1, IFI44L, | 0.873 | 0.863 | 0.884 | 0.923 | 0.865 | 0.982 | 0.935 | 0.854 | 1 |
| HERC6, FAM20A, RETN, MMP8 | | | | | | | | | |
| RSAD2, IFI27, OAS1, IFIT1, IFI44L, | 0.873 | 0.864 | 0.883 | 0.930 | 0.881 | 0.979 | 0.936 | 0.861 | 1 |
| SIGLEC1, HERC6, SLC1A2, FAM20A | | | | | | | | | |
| IFI27, OAS1, IFIT1, IFI44L, SLC1A2, | 0.873 | 0.863 | 0.883 | 0.929 | 0.875 | 0.983 | 0.921 | 0.839 | 1 |
| IL1R2, OLAH, FAM20A, RETN | | | | | | | | | |
| RSAD2, IFI27, SIGLEC1, ISG15, HERC6, | 0.873 | 0.863 | 0.884 | 0.933 | 0.886 | 0.981 | 0.943 | 0.873 | 1 |
| SLC1A2, IL1R2, FAM20A, RETN | | | | | | | | | |
| IFI27, IFIT1, IFI44L, SIGLEC1, ISG15, | 0.873 | 0.864 | 0.883 | 0.927 | 0.876 | 0.977 | 0.938 | 0.860 | 1 |
| HERC6, SLC1A2, FAM20A, MMP8 | | | | | | | | | |
| RSAD2, IFI27, SIGLEC1, ISG15, HERC6, | 0.873 | 0.864 | 0.883 | 0.930 | 0.881 | 0.980 | 0.950 | 0.897 | 1 |
| SLC1A2, OLAH, RETN, MMP8 | | | | | | | | | |
| RSAD2, IFI27, OAS1, IFIT1, IFI44L, | 0.873 | 0.864 | 0.882 | 0.923 | 0.873 | 0.974 | 0.949 | 0.899 | 0.999 |
| SIGLEC1, IL1R2, OLAH, MMP8 | | | | | | | | | |
| RSAD2, IFIT1, IFI44L, SIGLEC1, | 0.873 | 0.864 | 0.883 | 0.925 | 0.870 | 0.980 | 0.962 | 0.905 | 1 |
| HERC6, IL1R2, FAM20A, RETN, MMP8 | | | | | | | | | |
| IFI27, OAS1, IFIT1, IFI44L, SIGLEC1, | 0.873 | 0.864 | 0.883 | 0.930 | 0.881 | 0.979 | 0.937 | 0.862 | 1 |
| ISG15, HERC6, SLC1A2, FAM20A | | | | | | | | | |
| IFI27, OAS1, IFI44L, HERC6, SLC1A2, | 0.873 | 0.863 | 0.883 | 0.933 | 0.885 | 0.982 | 0.924 | 0.836 | 1 |
| IL1R2, FAM20A, RETN, MMP8 | | | | | | | | | |
| IFI27, IFIT1, IFI44L, HERC6, SLC1A2, | 0.873 | 0.864 | 0.883 | 0.932 | 0.883 | 0.981 | 0.932 | 0.849 | 1 |
| IL1R2, FAM20A, RETN, MMP8 | | | | | | | | | |
| OAS1, IFIT1, SIGLEC1, ISG15, IL1R2, | 0.873 | 0.864 | 0.882 | 0.920 | 0.863 | 0.978 | 0.968 | 0.918 | 1 |
| OLAH, FAM20A, RETN, MMP8 | | | | | | | | | |
| RSAD2, IFI27, OAS1, HERC6, SLC1A2, | 0.873 | 0.863 | 0.883 | 0.930 | 0.879 | 0.981 | 0.941 | 0.877 | 1 |
| IL1R2, OLAH, RETN, MMP8 | | | | | | | | | |
| RSAD2, IFI27, OAS1, SIGLEC1, ISG15, | 0.873 | 0.864 | 0.883 | 0.930 | 0.880 | 0.980 | 0.945 | 0.883 | 1 |
| HERC6, SLC1A2, OLAH, RETN | | | | | | | | | |
| RSAD2, OAS1, IFIT1, SIGLEC1, HERC6, | 0.873 | 0.864 | 0.883 | 0.921 | 0.864 | 0.978 | 0.950 | 0.892 | 1 |
| OLAH, FAM20A, RETN, MMP8 | | | | | | | | | |
| RSAD2, IFI27, OAS1, IFIT1, HERC6, | 0.873 | 0.863 | 0.883 | 0.927 | 0.876 | 0.979 | 0.940 | 0.875 | 1 |
| SLC1A2, OLAH, RETN, MMP8 | | | | | | | | | |
| RSAD2, IFI27, OAS1, IFI44L, ISG15, | 0.873 | 0.863 | 0.883 | 0.928 | 0.874 | 0.983 | 0.918 | 0.836 | 1 |
| SLC1A2, OLAH, FAM20A, RETN | | | | | | | | | |
| RSAD2, IFI27, IFIT1, HERC6, SLC1A2, | 0.873 | 0.863 | 0.883 | 0.933 | 0.884 | 0.982 | 0.933 | 0.852 | 1 |
| IL1R2, FAM20A, RETN, MMP8 | | | | | | | | | |
| IFI27, OAS1, IFIT1, IFI44L, SIGLEC1, | 0.873 | 0.864 | 0.882 | 0.920 | 0.862 | 0.978 | 0.952 | 0.896 | 1 |
| ISG15, OLAH, RETN, MMP8 | | | | | | | | | |
| RSAD2, IFI27, OAS1, IFI44L, SIGLEC1, | 0.873 | 0.864 | 0.883 | 0.924 | 0.870 | 0.979 | 0.959 | 0.907 | 1 |
| IL1R2, OLAH, RETN, MMP8 | | | | | | | | | |
| RSAD2, IFI27, OAS1, IFIT1, SLC1A2, | 0.873 | 0.864 | 0.882 | 0.927 | 0.873 | 0.981 | 0.920 | 0.836 | 1 |
| IL1R2, OLAH, FAM20A, RETN | | | | | | | | | |
| RSAD2, IFI27, IFIT1, IFI44L, SIGLEC1, | 0.873 | 0.864 | 0.882 | 0.925 | 0.871 | 0.979 | 0.955 | 0.904 | 1 |
| IL1R2, OLAH, RETN, MMP8 | | | | | | | | | |
| RSAD2, IFI27, OAS1, IFIT1, SIGLEC1, | 0.873 | 0.864 | 0.882 | 0.930 | 0.880 | 0.980 | 0.940 | 0.881 | 0.999 |
| ISG15, HERC6, SLC1A2, OLAH | | | | | | | | | |
| IFI27, IFIT1, IFI44L, SIGLEC1, ISG15, | 0.873 | 0.862 | 0.884 | 0.927 | 0.872 | 0.982 | 0.942 | 0.865 | 1 |
| SLC1A2, FAM20A, RETN, MMP8 | | | | | | | | | |
| IFI27, IFI44L, SIGLEC1, ISG15, HERC6, | 0.873 | 0.864 | 0.882 | 0.930 | 0.879 | 0.980 | 0.950 | 0.898 | 1 |
| SLC1A2, OLAH, RETN, MMP8 | | | | | | | | | |
| RSAD2, IFI27, IFIT1, IFI44L, ISG15, | 0.873 | 0.862 | 0.884 | 0.929 | 0.876 | 0.982 | 0.933 | 0.854 | 1 |
| IL1R2, FAM20A, RETN, MMP8 | | | | | | | | | |
| RSAD2, IFI27, IFI44L, SIGLEC1, | 0.873 | 0.864 | 0.882 | 0.930 | 0.880 | 0.980 | 0.951 | 0.899 | 1 |
| HERC6, SLC1A2, OLAH, RETN, MMP8 | | | | | | | | | |
| RSAD2, IFI27, OAS1, ISG15, SLC1A2, | 0.873 | 0.862 | 0.884 | 0.928 | 0.874 | 0.982 | 0.922 | 0.841 | 1 |
| OLAH, FAM20A, RETN, MMP8 | | | | | | | | | |
| RSAD2, IFIT1, IFI44L, SIGLEC1, ISG15, | 0.873 | 0.864 | 0.882 | 0.919 | 0.862 | 0.976 | 0.967 | 0.916 | 1 |
| IL1R2, OLAH, FAM20A, MMP8 | | | | | | | | | |
| IFI27, OAS1, ISG15, HERC6, SLC1A2, | 0.873 | 0.863 | 0.883 | 0.929 | 0.878 | 0.979 | 0.938 | 0.874 | 1 |
| IL1R2, OLAH, RETN, MMP8 | | | | | | | | | |
| RSAD2, IFIT1, SIGLEC1, HERC6, | 0.873 | 0.863 | 0.883 | 0.927 | 0.879 | 0.974 | 0.958 | 0.894 | 1 |
| SLC1A2, IL1R2, OLAH, FAM20A, MMP8 | | | | | | | | | |
| RSAD2, IFI27, OAS1, IFIT1, SIGLEC1, | 0.873 | 0.863 | 0.883 | 0.922 | 0.865 | 0.978 | 0.962 | 0.913 | 1 |
| IL1R2, OLAH, RETN, MMP8 | | | | | | | | | |
| IFI27, OAS1, IFIT1, IFI44L, HERC6, | 0.873 | 0.863 | 0.883 | 0.929 | 0.877 | 0.980 | 0.939 | 0.874 | 1 |
| SLC1A2, OLAH, RETN, MMP8 | | | | | | | | | |
| RSAD2, IFI27, IFIT1, IFI44L, SIGLEC1, | 0.873 | 0.864 | 0.882 | 0.925 | 0.873 | 0.977 | 0.945 | 0.892 | 0.997 |
| ISG15, HERC6, OLAH, MMP8 | | | | | | | | | |
| IFI27, OAS1, IFI44L, SIGLEC1, ISG15, | 0.873 | 0.863 | 0.882 | 0.931 | 0.882 | 0.979 | 0.937 | 0.873 | 1 |
| HERC6, SLC1A2, OLAH, RETN | | | | | | | | | |
| RSAD2, IFI27, OAS1, IFIT1, IFI44L, | 0.873 | 0.864 | 0.882 | 0.924 | 0.874 | 0.975 | 0.943 | 0.890 | 0.997 |
| SIGLEC1, ISG15, IL1R2, OLAH | | | | | | | | | |
| RSAD2, OAS1, IFIT1, SIGLEC1, ISG15, | 0.873 | 0.863 | 0.882 | 0.919 | 0.861 | 0.977 | 0.954 | 0.900 | 1 |
| HERC6, IL1R2, OLAH, FAM20A | | | | | | | | | |
| IFI27, OAS1, IFIT1, SIGLEC1, HERC6, | 0.873 | 0.863 | 0.883 | 0.929 | 0.881 | 0.977 | 0.942 | 0.885 | 0.999 |
| SLC1A2, IL1R2, OLAH, MMP8 | | | | | | | | | |
| RSAD2, IFI27, IFI44L, SIGLEC1, | 0.873 | 0.863 | 0.883 | 0.929 | 0.876 | 0.982 | 0.948 | 0.874 | 1 |
| HERC6, SLC1A2, FAM20A, RETN, MMP8 | | | | | | | | | |
| IFI27, OAS1, IFIT1, ISG15, HERC6, | 0.873 | 0.863 | 0.883 | 0.930 | 0.880 | 0.980 | 0.940 | 0.876 | 1 |
| SLC1A2, OLAH, RETN, MMP8 | | | | | | | | | |
| OAS1, IFIT1, IFI44L, SIGLEC1, ISG15, | 0.873 | 0.863 | 0.883 | 0.918 | 0.861 | 0.976 | 0.951 | 0.894 | 1 |
| HERC6, IL1R2, OLAH, FAM20A | | | | | | | | | |
| IFIT1, IFI44L, SIGLEC1, ISG15, IL1R2, | 0.873 | 0.863 | 0.882 | 0.920 | 0.863 | 0.978 | 0.967 | 0.916 | 1 |
| OLAH, FAM20A, RETN, MMP8 | | | | | | | | | |
| IFIT1, IFI44L, SIGLEC1, ISG15, HERC6, | 0.873 | 0.863 | 0.882 | 0.927 | 0.881 | 0.973 | 0.959 | 0.891 | 1 |
| SLC1A2, IL1R2, FAM20A, MMP8 | | | | | | | | | |
| IFI27, IFI44L, SIGLEC1, HERC6, | 0.873 | 0.863 | 0.882 | 0.929 | 0.879 | 0.979 | 0.955 | 0.906 | 1 |
| SLC1A2, IL1R2, OLAH, RETN, MMP8 | | | | | | | | | |
| RSAD2, IFI27, IFIT1, IFI44L, ISG15, | 0.873 | 0.864 | 0.882 | 0.923 | 0.870 | 0.977 | 0.937 | 0.878 | 0.996 |
| HERC6, IL1R2, OLAH, RETN | | | | | | | | | |
| RSAD2, IFI27, OAS1, IFIT1, SIGLEC1, | 0.873 | 0.863 | 0.882 | 0.929 | 0.880 | 0.977 | 0.946 | 0.889 | 1 |
| HERC6, SLC1A2, IL1R2, OLAH | | | | | | | | | |
| RSAD2, IFI27, OAS1, IFI44L, SIGLEC1, | 0.873 | 0.864 | 0.881 | 0.927 | 0.877 | 0.976 | 0.943 | 0.891 | 0.996 |
| ISG15, IL1R2, OLAH, MMP8 | | | | | | | | | |
| IFIT1, IFI44L, SIGLEC1, HERC6, | 0.873 | 0.862 | 0.883 | 0.930 | 0.881 | 0.979 | 0.948 | 0.883 | 1 |
| SLC1A2, OLAH, FAM20A, RETN, MMP8 | | | | | | | | | |
| IFI27, OAS1, IFIT1, SIGLEC1, ISG15, | 0.873 | 0.863 | 0.882 | 0.927 | 0.876 | 0.979 | 0.943 | 0.886 | 1 |
| HERC6, SLC1A2, RETN, MMP8 | | | | | | | | | |
| RSAD2, OAS1, IFIT1, IFI44L, SIGLEC1, | 0.873 | 0.863 | 0.883 | 0.917 | 0.859 | 0.975 | 0.949 | 0.891 | 1 |
| HERC6, OLAH, FAM20A, MMP8 | | | | | | | | | |
| RSAD2, OAS1, IFIT1, SIGLEC1, HERC6, | 0.873 | 0.862 | 0.883 | 0.926 | 0.876 | 0.975 | 0.943 | 0.874 | 1 |
| SLC1A2, OLAH, FAM20A, MMP8 | | | | | | | | | |
| OAS1, IFIT1, IFI44L, SIGLEC1, IL1R2, | 0.873 | 0.863 | 0.882 | 0.922 | 0.865 | 0.978 | 0.967 | 0.916 | 1 |
| OLAH, FAM20A, RETN, MMP8 | | | | | | | | | |
| RSAD2, IFI27, OAS1, SIGLEC1, ISG15, | 0.873 | 0.863 | 0.882 | 0.931 | 0.881 | 0.981 | 0.940 | 0.881 | 0.999 |
| HERC6, SLC1A2, OLAH, MMP8 | | | | | | | | | |
| RSAD2, IFI27, IFIT1, IFI44L, HERC6, | 0.873 | 0.863 | 0.882 | 0.930 | 0.884 | 0.977 | 0.928 | 0.842 | 1 |
| SLC1A2, IL1R2, FAM20A, MMP8 | | | | | | | | | |
| RSAD2, IFI27, IFI44L, SIGLEC1, ISG15, | 0.873 | 0.864 | 0.882 | 0.927 | 0.874 | 0.980 | 0.954 | 0.904 | 1 |
| IL1R2, OLAH, RETN, MMP8 | | | | | | | | | |
| RSAD2, IFI27, OAS1, SIGLEC1, ISG15, | 0.873 | 0.863 | 0.882 | 0.929 | 0.880 | 0.978 | 0.942 | 0.884 | 1 |
| HERC6, SLC1A2, IL1R2, OLAH | | | | | | | | | |
| RSAD2, IFI27, OAS1, IFIT1, ISG15, | 0.873 | 0.863 | 0.882 | 0.923 | 0.873 | 0.973 | 0.939 | 0.882 | 0.996 |
| HERC6, IL1R2, OLAH, MMP8 | | | | | | | | | |
| RSAD2, OAS1, IFIT1, IFI44L, SIGLEC1, | 0.873 | 0.864 | 0.882 | 0.919 | 0.862 | 0.976 | 0.955 | 0.901 | 1 |
| ISG15, HERC6, IL1R2, FAM20A | | | | | | | | | |
| IFI27, OAS1, IFIT1, IFI44L, SIGLEC1, | 0.873 | 0.864 | 0.881 | 0.920 | 0.865 | 0.975 | 0.950 | 0.898 | 1 |
| ISG15, IL1R2, OLAH, RETN | | | | | | | | | |
| IFI27, IFIT1, IFI44L, SIGLEC1, HERC6, | 0.873 | 0.863 | 0.882 | 0.929 | 0.878 | 0.980 | 0.953 | 0.904 | 1 |
| SLC1A2, OLAH, RETN, MMP8 | | | | | | | | | |
| RSAD2, IFI27, IFIT1, IFI44L, SIGLEC1, | 0.873 | 0.863 | 0.882 | 0.928 | 0.877 | 0.979 | 0.937 | 0.860 | 1 |
| HERC6, SLC1A2, FAM20A, MMP8 | | | | | | | | | |
| IFI27, IFI44L, SIGLEC1, ISG15, | 0.872 | 0.863 | 0.881 | 0.931 | 0.883 | 0.979 | 0.953 | 0.902 | 1 |
| SLC1A2, IL1R2, OLAH, RETN, MMP8 | | | | | | | | | |
| RSAD2, IFI27, OAS1, IFI44L, SIGLEC1, | 0.872 | 0.864 | 0.881 | 0.922 | 0.868 | 0.977 | 0.949 | 0.893 | 1 |
| ISG15, IL1R2, OLAH, RETN | | | | | | | | | |
| RSAD2, IFI27, OAS1, IFIT1, IFI44L, | 0.872 | 0.863 | 0.882 | 0.925 | 0.874 | 0.977 | 0.936 | 0.877 | 0.994 |
| ISG15, HERC6, OLAH, MMP8 | | | | | | | | | |
| RSAD2, IFIT1, IFI44L, SIGLEC1, | 0.872 | 0.863 | 0.882 | 0.927 | 0.878 | 0.975 | 0.959 | 0.904 | 1 |
| SLC1A2, IL1R2, OLAH, FAM20A, MMP8 | | | | | | | | | |
| RSAD2, IFIT1, IFI44L, SIGLEC1, | 0.872 | 0.863 | 0.882 | 0.929 | 0.884 | 0.974 | 0.959 | 0.893 | 1 |
| HERC6, SLC1A2, IL1R2, FAM20A, MMP8 | | | | | | | | | |
| OAS1, IFIT1, IFI44L, SIGLEC1, ISG15, | 0.872 | 0.862 | 0.883 | 0.924 | 0.872 | 0.977 | 0.941 | 0.877 | 1 |
| SLC1A2, OLAH, FAM20A, MMP8 | | | | | | | | | |
| RSAD2, IFI27, OAS1, SIGLEC1, ISG15, | 0.872 | 0.862 | 0.883 | 0.926 | 0.872 | 0.979 | 0.937 | 0.863 | 1 |
| HERC6, SLC1A2, FAM20A, RETN | | | | | | | | | |
| IFI27, OAS1, IFIT1, ISG15, HERC6, | 0.872 | 0.863 | 0.882 | 0.927 | 0.876 | 0.978 | 0.932 | 0.864 | 0.999 |
| SLC1A2, IL1R2, OLAH, RETN | | | | | | | | | |
| RSAD2, IFI27, IFIT1, IFI44L, SIGLEC1, | 0.872 | 0.863 | 0.881 | 0.929 | 0.879 | 0.978 | 0.943 | 0.888 | 0.999 |
| HERC6, SLC1A2, OLAH, RETN | | | | | | | | | |
| RSAD2, IFI27, OAS1, IFI44L, SLC1A2, | 0.872 | 0.861 | 0.883 | 0.929 | 0.874 | 0.983 | 0.927 | 0.851 | 1 |
| OLAH, FAM20A, RETN, MMP8 | | | | | | | | | |
| RSAD2, IFI27, OAS1, IFIT1, IFI44L, | 0.872 | 0.863 | 0.881 | 0.924 | 0.871 | 0.978 | 0.939 | 0.877 | 1 |
| HERC6, IL1R2, RETN, MMP8 | | | | | | | | | |
| RSAD2, IFI27, IFIT1, HERC6, SLC1A2, | 0.872 | 0.863 | 0.882 | 0.929 | 0.878 | 0.979 | 0.941 | 0.881 | 1 |
| IL1R2, OLAH, RETN, MMP8 | | | | | | | | | |
| IFI27, OAS1, IFIT1, IFI44L, SIGLEC1, | 0.872 | 0.862 | 0.882 | 0.927 | 0.877 | 0.977 | 0.941 | 0.884 | 0.999 |
| HERC6, SLC1A2, OLAH, MMP8 | | | | | | | | | |
| IFI27, IFIT1, SIGLEC1, ISG15, HERC6, | 0.872 | 0.863 | 0.882 | 0.928 | 0.877 | 0.979 | 0.940 | 0.882 | 0.998 |
| SLC1A2, IL1R2, OLAH, RETN | | | | | | | | | |
| IFI27, OAS1, SIGLEC1, ISG15, SLC1A2, | 0.872 | 0.863 | 0.882 | 0.930 | 0.881 | 0.979 | 0.953 | 0.898 | 1 |
| IL1R2, OLAH, RETN, MMP8 | | | | | | | | | |
| RSAD2, IFI27, IFIT1, IFI44L, SIGLEC1, | 0.872 | 0.864 | 0.881 | 0.922 | 0.869 | 0.976 | 0.945 | 0.893 | 0.996 |
| ISG15, HERC6, IL1R2, RETN | | | | | | | | | |
| RSAD2, IFI27, OAS1, IFIT1, IFI44L, | 0.872 | 0.864 | 0.881 | 0.926 | 0.873 | 0.979 | 0.941 | 0.876 | 1 |
| SIGLEC1, HERC6, SLC1A2, RETN | | | | | | | | | |
| RSAD2, IFI27, OAS1, IFIT1, SIGLEC1, | 0.872 | 0.862 | 0.882 | 0.927 | 0.872 | 0.982 | 0.943 | 0.869 | 1 |
| SLC1A2, FAM20A, RETN, MMP8 | | | | | | | | | |
| RSAD2, IFI27, OAS1, ISG15, HERC6, | 0.872 | 0.863 | 0.882 | 0.928 | 0.877 | 0.979 | 0.932 | 0.860 | 1 |
| SLC1A2, IL1R2, OLAH, RETN | | | | | | | | | |
| OAS1, IFIT1, IFI44L, SIGLEC1, ISG15, | 0.872 | 0.862 | 0.882 | 0.920 | 0.861 | 0.978 | 0.945 | 0.881 | 1 |
| HERC6, FAM20A, RETN, MMP8 | | | | | | | | | |
| RSAD2, IFI27, IFIT1, IFI44L, SLC1A2, | 0.872 | 0.862 | 0.882 | 0.927 | 0.872 | 0.981 | 0.922 | 0.841 | 1 |
| IL1R2, OLAH, FAM20A, RETN | | | | | | | | | |
| RSAD2, IFI27, IFI44L, SIGLEC1, ISG15, | 0.872 | 0.863 | 0.881 | 0.924 | 0.871 | 0.977 | 0.951 | 0.905 | 0.998 |
| HERC6, IL1R2, RETN, MMP8 | | | | | | | | | |
| RSAD2, IFI27, OAS1, IFI44L, SIGLEC1, | 0.872 | 0.863 | 0.882 | 10.930 | 0.881 | 0.980 | 0.943 | 0.886 | 1 |
| ISG15, HERC6, SLC1A2, OLAH | | | | | | | | | |
| RSAD2, IFI27, OAS1, IFI44L, SIGLEC1, | 0.872 | 0.862 | 0.883 | 0.930 | 0.881 | 0.979 | 0.943 | 0.873 | 1 |
| SLC1A2, IL1R2, FAM20A, RETN | | | | | | | | | |
| IFI27, OAS1, IFIT1, IFI44L, SIGLEC1, | 0.872 | 0.862 | 0.882 | 0.929 | 0.879 | 0.979 | 0.939 | 0.880 | 0.998 |
| ISG15, HERC6, SLC1A2, OLAH | | | | | | | | | |
| RSAD2, IFI27, SIGLEC1, ISG15, HERC6, | 0.872 | 0.863 | 0.881 | 0.929 | 0.880 | 0.978 | 0.941 | 0.885 | 0.997 |
| SLC1A2, IL1R2, OLAH, MMP8 | | | | | | | | | |
| RSAD2, IFI27, OAS1, IFIT1, IFI44L, | 0.872 | 0.863 | 0.881 | 0.923 | 0.870 | 0.976 | 0.938 | 0.875 | 1 |
| ISG15, HERC6, IL1R2, RETN | | | | | | | | | |
| RSAD2, IFIT1, IFI44L, SIGLEC1, ISG15, | 0.872 | 0.863 | 0.882 | 0.918 | 0.860 | 0.976 | 0.951 | 0.894 | 1 |
| HERC6, IL1R2, OLAH, FAM20A | | | | | | | | | |
| RSAD2, IFI27, OAS1, HERC6, SLC1A2, | 0.872 | 0.862 | 0.882 | 0.935 | 0.888 | 0.982 | 0.925 | 0.839 | 1 |
| IL1R2, FAM20A, RETN, MMP8 | | | | | | | | | |
| RSAD2, IFI27, IFIT1, SIGLEC1, ISG15, | 0.872 | 0.862 | 0.882 | 0.926 | 0.871 | 0.982 | 0.943 | 0.868 | 1 |
| SLC1A2, FAM20A, RETN, MMP8 | | | | | | | | | |
| RSAD2, IFI27, IFIT1, IFI44L, SIGLEC1, | 0.872 | 0.864 | 0.881 | 0.925 | 0.875 | 0.975 | 0.943 | 0.891 | 0.996 |
| ISG15, IL1R2, OLAH, MMP8 | | | | | | | | | |
| OAS1, IFIT1, IFI44L, SIGLEC1, | 0.872 | 0.862 | 0.882 | 0.927 | 0.89 | 0.975 | 0.959 | 0.904 | 1 |
| SLC1A2, IL1R2, OLAH, FAM20A, MMP8 | | | | | | | | | |
| RSAD2, IFI27, IFIT1, IFI44L, SIGLEC1, | 0.872 | 0.862 | 0.882 | 0.926 | 0.871 | 0.982 | 0.943 | 0.870 | 1 |
| SLC1A2, FAM20A, RETN, MMP8 | | | | | | | | | |
| IFI27, IFIT1, SIGLEC1, ISG15, HERC6, | 0.872 | 0.862 | 0.882 | 0.930 | 0.880 | 0.981 | 0.952 | 0.900 | 1 |
| SLC1A2, OLAH, RETN, MMP8 | | | | | | | | | |
| IFI27, IFI44L, SIGLEC1, ISG15, HERC6, | 0.872 | 0.863 | 0.881 | 0.928 | 0.878 | 0.978 | 0.943 | 0.887 | 1 |
| SLC1A2, IL1R2, OLAH, RETN | | | | | | | | | |
| RSAD2, OAS1, IFIT1, SIGLEC1, IL1R2, | 0.872 | 0.863 | 0.882 | 0.921 | 0.864 | 0.978 | 0.966 | 0.915 | 1 |
| OLAH, FAM20A, RETN, MMP8 | | | | | | | | | |
| RSAD2, IFI27, OAS1, IFIT1, ISG15, | 0.872 | 0.863 | 0.881 | 0.925 | 0.871 | 0.978 | 0.945 | 0.885 | 1 |
| HERC6, IL1R2, RETN, MMP8 | | | | | | | | | |
| RSAD2, IFI27, OAS1, IFIT1, IFI44L, | 0.872 | 0.863 | 0.881 | 0.923 | 0.873 | 0.974 | 0.936 | 0.878 | 0.994 |
| ISG15, HERC6, IL1R2, OLAH | | | | | | | | | |
| IFI27, OAS1, IFIT1, SIGLEC1, ISG15, | 0.872 | 0.863 | 0.881 | 0.928 | 0.877 | 0.979 | 0.942 | 0.881 | 1 |
| SLC1A2, IL1R2, OLAH, RETN | | | | | | | | | |
| IFIT1, IFI44L, SIGLEC1, ISG15, HERC6, | 0.872 | 0.863 | 0.881 | 0.918 | 0.860 | 0.976 | 0.951 | 0.894 | 1 |
| IL1R2, OLAH, FAM20A, RETN | | | | | | | | | |
| RSAD2, OAS1, SIGLEC1, HERC6, | 0.872 | 0.862 | 0.882 | 10.924 | 0.876 | 0.972 | 0.960 | 0.900 | 1 |
| SLC1A2, IL1R2, OLAH, FAM20A, MMP8 | | | | | | | | | |
| IFI27, IFIT1, IFI44L, SIGLEC1, ISG15, | 0.872 | 0.863 | 0.881 | 0.928 | 0.879 | 0.977 | 0.943 | 0.890 | 0.997 |
| SLC1A2, IL1R2, OLAH, MMP8 | | | | | | | | | |
| RSAD2, IFI27, OAS1, ISG15, HERC6, | 0.872 | 0.862 | 0.882 | 0.930 | 0.879 | 0.981 | 0.940 | 0.875 | 1 |
| SLC1A2, OLAH, RETN, MMP8 | | | | | | | | | |
| RSAD2, IFI27, OAS1, SIGLEC1, HERC6, | 0.872 | 0.862 | 0.882 | 0.928 | 0.879 | 0.977 | 0.946 | 0.889 | 1 |
| SLC1A2, IL1R2, OLAH, MMP8 | | | | | | | | | |
| RSAD2, IFI27, OAS1, IFIT1, SIGLEC1, | 0.872 | 0.862 | 0.882 | 0.928 | 0.878 | 0.978 | 0.940 | 0.881 | 0.999 |
| HERC6, SLC1A2, OLAH, MMP8 | | | | | | | | | |
| RSAD2, IFI27, IFIT1, SIGLEC1, ISG15, | 0.872 | 0.863 | 0.881 | 0.925 | 0.873 | 0.977 | 0.961 | 0.920 | 1 |
| HERC6, IL1R2, RETN, MMP8 | | | | | | | | | |
| IFI27, SIGLEC1, ISG15, HERC6, | 0.872 | 0.862 | 0.881 | 0.929 | 0.878 | 0.979 | 0.951 | 0.899 | 1 |
| SLC1A2, IL1R2, OLAH, RETN, MMP8 | | | | | | | | | |
| IFI27, OAS1, IFIT1, IFI44L, SIGLEC1, | 0.872 | 0.863 | 0.881 | 0.926 | 0.876 | 0.977 | 0.942 | 0.884 | 1 |
| ISG15, HERC6, SLC1A2, RETN | | | | | | | | | |
| IFIT1, SIGLEC1, HERC6, SLC1A2, | 0.872 | 0.862 | 0.882 | 0.928 | 0.881 | 0.976 | 0.959 | 0.893 | 1 |
| IL1R2, OLAH, FAM20A, RETN, MMP8 | | | | | | | | | |
| RSAD2, IFI27, OAS1, IFIT1, IFI44L, | 0.872 | 0.863 | 0.881 | 0.920 | 0.864 | 0.975 | 0.953 | 0.897 | 1 |
| SIGLEC1, IL1R2, OLAH, RETN | | | | | | | | | |
| RSAD2, IFI27, OAS1, IFIT1, SIGLEC1, | 0.872 | 0.862 | 0.881 | 0.920 | 0.861 | 0.979 | 0.959 | 0.907 | 1 |
| ISG15, OLAH, RETN, MMP8 | | | | | | | | | |
| IFI27, OAS1, IFIT1, SIGLEC1, SLC1A2, | 0.872 | 0.862 | 0.882 | 0.928 | 0.878 | 0.978 | 0.955 | 0.900 | 1 |
| IL1R2, OLAH, RETN, MMP8 | | | | | | | | | |
| IFI27, OAS1, IFIT1, IFI44L, ISG15, | 0.872 | 0.863 | 0.881 | 0.924 | 0.874 | 0.975 | 0.939 | 0.884 | 0.995 |
| HERC6, IL1R2, OLAH, MMP8 | | | | | | | | | |
| RSAD2, OAS1, SIGLEC1, ISG15, | 0.872 | 0.862 | 0.881 | 0.920 | 0.863 | 0.977 | 0.958 | 0.896 | 1 |
| HERC6, IL1R2, FAM20A, RETN, MMP8 | | | | | | | | | |
| RSAD2, IFI27, OAS1, IFIT1, SIGLEC1, | 0.872 | 0.862 | 0.881 | 0.929 | 0.880 | 0.978 | 0.937 | 0.862 | 1 |
| HERC6, SLC1A2, FAM20A, MMP8 | | | | | | | | | |
| IFIT1, IFI44L, SIGLEC1, HERC6, | 0.872 | 0.862 | 0.881 | 0.930 | 0.883 | 0.976 | 0.960 | 0.895 | 1 |
| SLC1A2, IL1R2, FAM20A, RETN, MMP8 | | | | | | | | | |
| IFI27, OAS1, IFIT1, SIGLEC1, ISG15, | 0.872 | 0.863 | 0.881 | 0.929 | 0.880 | 0.978 | 0.939 | 0.880 | 0.999 |
| SLC1A2, IL1R2, OLAH, MMP8 | | | | | | | | | |
| OAS1, IFIT1, SIGLEC1, HERC6, | 0.872 | 0.862 | 0.882 | 0.926 | 0.876 | 0.976 | 0.947 | 0.880 | 1 |
| SLC1A2, OLAH, FAM20A, RETN, MMP8 | | | | | | | | | |
| RSAD2, OAS1, IFIT1, IFI44L, SIGLEC1, | 0.872 | 0.862 | 0.882 | 0.920 | 0.862 | 0.977 | 0.967 | 0.914 | 1 |
| ISG15, IL1R2, FAM20A, MMP8 | | | | | | | | | |
| RSAD2, IFI27, OAS1, SIGLEC1, ISG15, | 0.872 | 0.862 | 0.882 | 0.924 | 0.869 | 0.979 | 0.959 | 0.910 | 1 |
| IL1R2, OLAH, RETN, MMP8 | | | | | | | | | |
| RSAD2, IFIT1, IFI44L, SIGLEC1, ISG15, | 0.872 | 0.862 | 0.882 | 0.925 | 0.873 | 0.977 | 0.940 | 0.875 | 1 |
| SLC1A2, OLAH, FAM20A, MMP8 | | | | | | | | | |
| RSAD2, IFI27, IFI44L, HERC6, SLC1A2, | 0.872 | 0.862 | 0.882 | 0.933 | 0.884 | 0.981 | 0.926 | 0.841 | 1 |
| IL1R2, FAM20A, RETN, MMP8 | | | | | | | | | |
| IFI27, IFIT1, SIGLEC1, ISG15, HERC6, | 0.872 | 0.862 | 0.881 | 0.929 | 0.880 | 0.978 | 0.940 | 0.884 | 0.997 |
| SLC1A2, IL1R2, OLAH, MMP8 | | | | | | | | | |
| IFI27, OAS1, IFI44L, SIGLEC1, HERC6, | 0.872 | 0.862 | 0.882 | 0.930 | 0.882 | 0.979 | 0.939 | 0.876 | 1 |
| SLC1A2, IL1R2, OLAH, RETN | | | | | | | | | |
| OAS1, SIGLEC1, ISG15, HERC6, IL1R2, | 0.872 | 0.862 | 0.881 | 0.917 | 0.859 | 0.975 | 0.963 | 0.909 | 1 |
| OLAH, FAM20A, RETN, MMP8 | | | | | | | | | |
| OAS1, IFIT1, SIGLEC1, HERC6, | 0.872 | 0.862 | 0.882 | 0.929 | 0.882 | 0.975 | 0.955 | 0.890 | 1 |
| SLC1A2, IL1R2, FAM20A, RETN, MMP8 | | | | | | | | | |
| RSAD2, IFI27, OAS1, IFI44L, SIGLEC1, | 0.872 | 0.863 | 0.881 | 0.920 | 0.862 | 0.978 | 0.959 | 0.908 | 1 |
| ISG15, OLAH, RETN, MMP8 | | | | | | | | | |
| OAS1, IFI44L, SIGLEC1, ISG15, HERC6, | 0.872 | 0.862 | 0.882 | 0.918 | 0.861 | 0.975 | 0.962 | 0.906 | 1 |
| IL1R2, OLAH, FAM20A, MMP8 | | | | | | | | | |
| OAS1, IFIT1, SIGLEC1, ISG15, HERC6, | 0.872 | 0.862 | 0.882 | 0.929 | 0.883 | 0.974 | 0.959 | 0.893 | 1 |
| SLC1A2, IL1R2, FAM20A, MMP8 | | | | | | | | | |
| IFI27, IFIT1, IFI44L, SIGLEC1, ISG15, | 0.872 | 0.863 | 0.881 | 0.927 | 0.877 | 0.978 | 0.941 | 0.884 | 0.998 |
| HERC6, SLC1A2, OLAH, RETN | | | | | | | | | |
| IFI27, OAS1, IFIT1, SIGLEC1, ISG15, | 0.872 | 0.862 | 0.882 | 0.930 | 0.881 | 0.978 | 0.936 | 0.860 | 1 |
| HERC6, SLC1A2, FAM20A, MMP8 | | | | | | | | | |
| OAS1, IFIT1, IFI44L, SIGLEC1, ISG15, | 0.872 | 0.862 | 0.881 | 0.923 | 0.866 | 0.979 | 0.967 | 0.914 | 1 |
| IL1R2, FAM20A, RETN, MMP8 | | | | | | | | | |
| RSAD2, OAS1, IFIT1, SIGLEC1, HERC6, | 0.872 | 0.862 | 0.881 | 0.927 | 0.881 | 0.973 | 0.959 | 0.895 | 1 |
| SLC1A2, IL1R2, FAM20A, MMP8 | | | | | | | | | |
| IFI27, OAS1, IFIT1, IFI44L, SIGLEC1, | 0.871 | 0.862 | 0.881 | 0.930 | 0.880 | 0.980 | 0.943 | 0.886 | 1 |
| ISG15, SLC1A2, OLAH, MMP8 | | | | | | | | | |
| IFI27, OAS1, SIGLEC1, ISG15, HERC6, | 0.871 | 0.862 | 0.880 | 0.928 | 0.881 | 0.976 | 0.957 | 0.905 | 1 |
| SLC1A2, IL1R2, RETN, MMP8 | | | | | | | | | |
| RSAD2, IFI27, OAS1, IFIT1, SIGLEC1, | 0.871 | 0.863 | 0.880 | 0.924 | 0.870 | 0.978 | 0.939 | 0.876 | 1 |
| ISG15, HERC6, SLC1A2, RETN | | | | | | | | | |
| IFI27, OAS1, IFIT1, ISG15, HERC6, | 0.871 | 0.861 | 0.882 | 0.934 | 0.887 | 0.981 | 0.926 | 0.842 | 1 |
| SLC1A2, IL1R2, FAM20A, RETN | | | | | | | | | |
| OAS1, IFIT1, SIGLEC1, ISG15, HERC6, | 0.871 | 0.861 | 0.882 | 0.926 | 0.877 | 0.975 | 0.943 | 0.873 | 1 |
| SLC1A2, OLAH, FAM20A, MMP8 | | | | | | | | | |
| IFI27, OAS1, IFI44L, SIGLEC1, ISG15, | 0.871 | 0.861 | 0.881 | 0.930 | 0.880 | 0.979 | 0.939 | 0.881 | 0.997 |
| HERC6, SLC1A2, OLAH, MMP8 | | | | | | | | | |
| RSAD2, IFI27, IFIT1, IFI44L, SIGLEC1, | 0.871 | 0.862 | 0.881 | 0.920 | 0.862 | 0.978 | 0.953 | 0.898 | 1 |
| ISG15, OLAH, RETN, MMP8 | | | | | | | | | |
| RSAD2, IFI27, OAS1, IFIT1, SIGLEC1, | 0.871 | 0.862 | 0.881 | 0.929 | 0.878 | 0.980 | 0.943 | 0.886 | 1 |
| ISG15, SLC1A2, OLAH, MMP8 | | | | | | | | | |
| RSAD2, IFI27, IFIT1, IFI44L, SIGLEC1, | 0.871 | 0.862 | 0.881 | 0.926 | 0.871 | 0.981 | 0.937 | 0.861 | 1 |
| ISG15, SLC1A2, FAM20A, RETN | | | | | | | | | |
| RSAD2, IFI27, OAS1, IFIT1, HERC6, | 0.871 | 0.861 | 0.882 | 0.930 | 0.884 | 0.976 | 0.927 | 0.842 | 1 |
| SLC1A2, IL1R2, FAM20A, MMP8 | | | | | | | | | |
| IFI44L, SIGLEC1, ISG15, HERC6, | 0.871 | 0.861 | 0.881 | 0.917 | 0.859 | 0.975 | 0.960 | 0.900 | 1 |
| IL1R2, OLAH, FAM20A, RETN, MMP8 | | | | | | | | | |
| RSAD2, IFI27, OAS1, IFIT1, ISG15, | 0.871 | 0.861 | 0.882 | 0.925 | 0.870 | 0.980 | 0.923 | 0.843 | 1 |
| SLC1A2, OLAH, FAM20A, RETN | | | | | | | | | |
| IFI27, IFI44L, SIGLEC1, ISG15, HERC6, | 0.871 | 0.862 | 0.881 | 0.930 | 0.881 | 0.978 | 0.943 | 0.890 | 0.997 |
| SLC1A2, IL1R2, OLAH, MMP8 | | | | | | | | | |
| IFI27, OAS1, IFI44L, ISG15, HERC6, | 0.871 | 0.861 | 0.882 | 0.932 | 0.885 | 0.980 | 0.925 | 0.840 | 1 |
| SLC1A2, IL1R2, FAM20A, RETN | | | | | | | | | |
| IFI27, OAS1, IFIT1, IFI44L, HERC6, | 0.871 | 0.861 | 0.881 | 0.932 | 0.885 | 0.980 | 0.925 | 0.840 | 1 |
| SLC1A2, IL1R2, FAM20A, RETN | | | | | | | | | |
| RSAD2, IFI27, OAS1, IFIT1, IFI44L, | 0.871 | 0.862 | 0.881 | 0.922 | 0.867 | 0.977 | 0.950 | 0.895 | 1 |
| IL1R2, OLAH, RETN, MMP8 | | | | | | | | | |
| RSAD2, IFI27, ISG15, HERC6, SLC1A2, | 0.871 | 0.862 | 0.881 | 0.933 | 0.885 | 0.981 | 0.929 | 0.844 | 1 |
| IL1R2, FAM20A, RETN, MMP8 | | | | | | | | | |
| IFI27, OAS1, IFIT1, SIGLEC1, ISG15, | 0.871 | 0.862 | 0.881 | 0.928 | 0.881 | 0.975 | 0.948 | 0.892 | 1 |
| HERC6, SLC1A2, IL1R2, RETN | | | | | | | | | |
| RSAD2, OAS1, IFIT1, SIGLEC1, ISG15, | 0.871 | 0.861 | 0.882 | 0.916 | 0.858 | 0.975 | 0.952 | 0.896 | 1 |
| HERC6, OLAH, FAM20A, MMP8 | | | | | | | | | |
| IFIT1, SIGLEC1, ISG15, HERC6, | 0.871 | 0.862 | 0.881 | 0.926 | 0.878 | 0.974 | 0.957 | 0.889 | 1 |
| SLC1A2, IL1R2, FAM20A, RETN, MMP8 | | | | | | | | | |
| RSAD2, IFI27, OAS1, IFI44L, SIGLEC1, | 0.871 | 0.862 | 0.881 | 0.929 | 0.879 | 0.979 | 0.947 | 0.892 | 1 |
| HERC6, SLC1A2, OLAH, MMP8 | | | | | | | | | |
| IFI27, OAS1, IFI44L, SIGLEC1, HERC6, | 0.871 | 0.862 | 0.881 | 0.927 | 0.879 | 0.975 | 0.943 | 0.888 | 0.999 |
| SLC1A2, IL1R2, OLAH, MMP8 | | | | | | | | | |
| RSAD2, IFI27, OAS1, IFI44L, SIGLEC1, | 0.871 | 0.863 | 0.880 | 0.927 | 0.879 | 0.976 | 0.946 | 0.884 | 1 |
| HERC6, SLC1A2, IL1R2, RETN | | | | | | | | | |
| RSAD2, IFI27, IFIT1, SIGLEC1, ISG15, | 0.871 | 0.862 | 0.881 | 0.929 | 0.879 | 0.978 | 0.941 | 0.884 | 0.999 |
| HERC6, SLC1A2, OLAH, RETN | | | | | | | | | |
| RSAD2, IFI27, OAS1, IFI44L, SIGLEC1, | 0.871 | 0.862 | 0.881 | 0.928 | 0.880 | 0.976 | 0.950 | 0.897 | 1 |
| HERC6, SLC1A2, IL1R2, OLAH | | | | | | | | | |
| RSAD2, IFI27, IFI44L, HERC6, SLC1A2, | 0.871 | 0.862 | 0.881 | 0.928 | 0.877 | 0.979 | 0.941 | 0.881 | 1 |
| IL1R2, OLAH, RETN, MMP8 | | | | | | | | | |
| RSAD2, IFI27, IFI44L, SIGLEC1, ISG15, | 0.871 | 0.862 | 0.880 | 0.927 | 0.876 | 0.978 | 0.943 | 0.887 | 1 |
| HERC6, SLC1A2, OLAH, RETN | | | | | | | | | |
| RSAD2, IFIT1, IFI44L, SIGLEC1, | 0.871 | 0.862 | 0.881 | 0.921 | 0.863 | 0.979 | 0.949 | 0.890 | 1 |
| HERC6, OLAH, FAM20A, RETN, MMP8 | | | | | | | | | |
| IFI27, IFIT1, IFI44L, SIGLEC1, ISG15, | 0.871 | 0.862 | 0.881 | 0.930 | 0.880 | 0.979 | 0.940 | 0.882 | 0.998 |
| HERC6, SLC1A2, OLAH, MMP8 | | | | | | | | | |
| IFI27, OAS1, IFIT1, IFI44L, ISG15, | 0.871 | 0.861 | 0.881 | 0.931 | 0.886 | 0.977 | 0.926 | 0.840 | 1 |
| HERC6, SLC1A2, IL1R2, FAM20A | | | | | | | | | |
| RSAD2, IFI27, OAS1, IFIT1, IFI44L, | 0.871 | 0.862 | 0.881 | 0.929 | 0.878 | 0.979 | 0.947 | 0.892 | 1 |
| SIGLEC1, HERC6, SLC1A2, OLAH | | | | | | | | | |
| RSAD2, IFI27, IFIT1, IFI44L, ISG15, | 0.871 | 0.862 | 0.880 | 0.925 | 0.875 | 0.975 | 0.937 | 0.880 | 0.993 |
| HERC6, IL1R2, OLAH, MMP8 | | | | | | | | | |
| RSAD2, IFI27, ISG15, HERC6, SLC1A2, | 0.871 | 0.862 | 0.881 | 0.928 | 0.878 | 0.979 | 0.940 | 0.879 | 1 |
| IL1R2, OLAH, RETN, MMP8 | | | | | | | | | |
| RSAD2, IFI27, OAS1, IFIT1, SIGLEC1, | 0.871 | 0.862 | 0.880 | 0.924 | 0.870 | 0.979 | 0.942 | 0.880 | 1 |
| HERC6, SLC1A2, RETN, MMP8 | | | | | | | | | |
| RSAD2, IFI27, OAS1, SIGLEC1, HERC6, | 0.871 | 0.862 | 0.881 | 0.927 | 0.879 | 0.976 | 0.960 | 0.909 | 1 |
| SLC1A2, IL1R2, RETN, MMP8 | | | | | | | | | |
| RSAD2, IFI27, OAS1, IFIT1, SIGLEC1, | 0.871 | 0.862 | 0.880 | 0.929 | 0.879 | 0.979 | 0.942 | 0.884 | 1 |
| ISG15, SLC1A2, IL1R2, OLAH | | | | | | | | | |
| RSAD2, IFIT1, SIGLEC1, ISG15, | 0.871 | 0.862 | 0.881 | 0.927 | 0.881 | 0.973 | 0.958 | 0.890 | 1 |
| HERC6, SLC1A2, IL1R2, FAM20A, MMP8 | | | | | | | | | |
| IFI27, OAS1, IFI44L, SIGLEC1, HERC6, | 0.871 | 0.862 | 0.880 | 0.928 | 0.881 | 0.975 | 0.955 | 0.906 | 1 |
| SLC1A2, IL1R2, RETN, MMP8 | | | | | | | | | |
| RSAD2, IFI27, IFI44L, SIGLEC1, | 0.871 | 0.862 | 0.880 | 0.928 | 0.879 | 0.976 | 0.949 | 0.898 | 0.999 |
| HERC6, SLC1A2, IL1R2, OLAH, MMP8 | | | | | | | | | |
| IFI27, OAS1, IFIT1, IFI44L, ISG15, | 0.871 | 0.862 | 0.880 | 0.924 | 0.871 | 0.977 | 0.940 | 0.880 | 1 |
| HERC6, IL1R2, RETN, MMP8 | | | | | | | | | |
| RSAD2, IFIT1, SIGLEC1, ISG15, | 0.871 | 0.862 | 0.881 | 0.921 | 0.863 | 0.978 | 0.952 | 0.896 | 1 |
| HERC6, OLAH, FAM20A, RETN, MMP8 | | | | | | | | | |
| RSAD2, IFI27, OAS1, IFI44L, SLC1A2, | 0.871 | 0.861 | 0.881 | 0.929 | 0.875 | 0.984 | 0.921 | 0.838 | 1 |
| IL1R2, OLAH, FAM20A, RETN | | | | | | | | | |
| IFIT1, SIGLEC1, ISG15, HERC6, | 0.871 | 0.861 | 0.881 | 0.928 | 0.878 | 0.977 | 0.950 | 0.883 | 1 |
| SLC1A2, OLAH, FAM20A, RETN, MMP8 | | | | | | | | | |
| IFI27, OAS1, IFIT1, ISG15, HERC6, | 0.871 | 0.861 | 0.881 | 0.928 | 0.879 | 0.977 | 0.929 | 0.862 | 0.996 |
| SLC1A2, IL1R2, OLAH, MMP8 | | | | | | | | | |
| IFI27, IFIT1, IFI44L, SIGLEC1, ISG15, | 0.871 | 0.862 | 0.880 | 0.930 | 0.879 | 0.980 | 0.952 | 0.897 | 1 |
| SLC1A2, OLAH, RETN, MMP8 | | | | | | | | | |
| IFI27, IFIT1, IFI44L, SIGLEC1, HERC6, | 0.871 | 0.862 | 0.881 | 0.927 | 0.878 | 0.977 | 0.945 | 0.889 | 1 |
| SLC1A2, IL1R2, OLAH, RETN | | | | | | | | | |
| RSAD2, IFI27, IFIT1, IFI44L, SIGLEC1, | 0.871 | 0.863 | 0.880 | 0.922 | 0.867 | 0.977 | 0.950 | 0.898 | 1 |
| ISG15, IL1R2, OLAH, RETN | | | | | | | | | |
| IFI27, IFIT1, IFI44L, ISG15, SLC1A2, | 0.871 | 0.861 | 0.881 | 0.931 | 0.881 | 0.980 | 0.933 | 0.851 | 1 |
| IL1R2, FAM20A, RETN, MMP8 | | | | | | | | | |
| RSAD2, IFI27, IFIT1, SIGLEC1, | 0.871 | 0.862 | 0.881 | 0.930 | 0.881 | 0.979 | 0.959 | 0.908 | 1 |
| SLC1A2, IL1R2, OLAH, RETN, MMP8 | | | | | | | | | |
| RSAD2, IFI27, OAS1, IFIT1, IFI44L, | 0.871 | 0.861 | 0.88] | 0.915 | 0.851 | 0.980 | 0.943 | 0.873 | 1 |
| SIGLEC1, ISG15, FAM20A, MMP8 | | | | | | | | | |
| OAS1, IFIT1, IFI44L, SIGLEC1, HERC6, | 0.871 | 0.862 | 0.880 | 0.916 | 0.858 | 0.975 | 0.951 | 0.894 | 1 |
| IL1R2, OLAH, FAM20A, RETN | | | | | | | | | |
| RSAD2, IFI27, OAS1, IFIT1, IFI44L, | 0.871 | 0.862 | 0.880 | 0.920 | 0.863 | 0.976 | 0.939 | 0.882 | 0.997 |
| SIGLEC1, ISG15, OLAH, MMP8 | | | | | | | | | |
| RSAD2, SIGLEC1, ISG15, HERC6, | 0.871 | 0.861 | 0.881 | 0.919 | 0.862 | 0.976 | 0.961 | 0.905 | 1 |
| IL1R2, OLAH, FAM20A, RETN, MMP8 | | | | | | | | | |
| IFI27, OAS1, IFIT1, IFI44L, SIGLEC1, | 0.871 | 0.862 | 0.880 | 0.927 | 0.876 | 0.978 | 0.945 | 0.887 | 1 |
| HERC6, SLC1A2, RETN, MMP8 | | | | | | | | | |
| RSAD2, IFIT1, SIGLEC1, ISG15, | 0.871 | 0.862 | 0.880 | 0.926 | 0.878 | 0.974 | 0.962 | 0.900 | 1 |
| SLC1A2, IL1R2, OLAH, FAM20A, MMP8 | | | | | | | | | |
| RSAD2, IFI27, OAS1, IFI44L, ISG15, | 0.871 | 0.860 | 0.882 | 0.930 | 0.878 | 0.982 | 0.935 | 0.856 | 1 |
| IL1R2, FAM20A, RETN, MMP8 | | | | | | | | | |
| RSAD2, OAS1, IFIT1, SIGLEC1, ISG15, | 0.871 | 0.862 | 0.880 | 0.922 | 0.865 | 0.979 | 0.967 | 0.914 | 1 |
| IL1R2, FAM20A, RETN, MMP8 | | | | | | | | | |
| RSAD2, IFI27, OAS1, IFIT1, IFI44L, | 0.871 | 0.861 | 0.881 | 0.924 | 0.871 | 0.977 | 0.930 | 0.852 | 1 |
| ISG15, IL1R2, FAM20A, RETN | | | | | | | | | |
| RSAD2, IFI27, OAS1, IFIT1, SIGLEC1, | 0.871 | 0.862 | 0.880 | 0.928 | 0.879 | 0.977 | 0.936 | 0.859 | 1 |
| ISG15, HERC6, SLC1A2, FAM20A | | | | | | | | | |
| IFI27, OAS1, IFIT1, IFI44L, SIGLEC1, | 0.871 | 0.861 | 0.881 | 0.927 | 0.875 | 0.979 | 0.957 | 0.901 | 1 |
| SLC1A2, OLAH, RETN, MMP8 | | | | | | | | | |
| RSAD2, IFI27, OAS1, IFIT1, SIGLEC1, | 0.871 | 0.861 | 0.880 | 0.925 | 0.869 | 0.980 | 0.938 | 0.863 | 1 |
| ISG15, SLC1A2, FAM20A, RETN | | | | | | | | | |
| RSAD2, SIGLEC1, ISG15, HERC6, | 0.871 | 0.861 | 0.881 | 0.925 | 0.877 | 0.973 | 0.961 | 0.901 | 1 |
| SLC1A2, IL1R2, OLAH, FAM20A, MMP8 | | | | | | | | | |
| RSAD2, IFI27, IFIT1, ISG15, HERC6, | 0.871 | 0.861 | 0.881 | 0.929 | 0.879 | 0.979 | 0.941 | 0.881 | 1 |
| SLC1A2, OLAH, RETN, MMP8 | | | | | | | | | |
| RSAD2, IFI27, OAS1, IFI44L, ISG15, | 0.871 | 0.862 | 0.880 | 0.923 | 0.869 | 0.976 | 0.940 | 0.877 | 1 |
| HERC6, IL1R2, RETN, MMP8 | | | | | | | | | |
| OAS1, IFIT1, SIGLEC1, ISG15, SLC1A2, | 0.871 | 0.861 | 0.880 | 0.926 | 0.879 | 0.974 | 0.961 | 0.899 | 1 |
| IL1R2, OLAH, FAM20A, MMP8 | | | | | | | | | |
| RSAD2, IFI27, OAS1, SIGLEC1, ISG15, | 0.871 | 0.862 | 0.880 | 0.930 | 0.879 | 0.981 | 0.948 | 0.885 | 1 |
| SLC1A2, IL1R2, OLAH, RETN | | | | | | | | | |
| RSAD2, IFI27, IFIT1, SIGLEC1, ISG15, | 0.871 | 0.862 | 0.880 | 0.929 | 0.880 | 0.978 | 0.940 | 0.883 | 0.998 |
| HERC6, SLC1A2, IL1R2, OLAH | | | | | | | | | |
| RSAD2, IFI27, OAS1, IFIT1, IFI44L, | 0.871 | 0.861 | 0.880 | 0.930 | 0.884 | 0.976 | 0.926 | 0.840 | 1 |
| HERC6, SLC1A2, IL1R2, FAM20A | | | | | | | | | |
| IFI27, IFI44L, ISG15, HERC6, SLC1A2, | 0.871 | 0.861 | 0.880 | 0.929 | 0.879 | 0.979 | 0.938 | 0.875 | 1 |
| IL1R2, OLAH, RETN, MMP8 | | | | | | | | | |
| RSAD2, IFI27, IFI44L, SIGLEC1, ISG15, | 0.871 | 0.862 | 0.879 | 0.929 | 0.879 | 0.978 | 0.945 | 0.891 | 0.998 |
| SLC1A2, IL1R2, OLAH, MMP8 | | | | | | | | | |
| RSAD2, IFI27, OAS1, IFIT1, IFI44L, | 0.871 | 0.861 | 0.881 | 0.927 | 0.872 | 0.981 | 0.940 | 0.868 | 1 |
| SIGLEC1, SLC1A2, FAM20A, RETN | | | | | | | | | |
| RSAD2, IFI27, OAS1, IFIT1, IFI44L, | 0.871 | 0.861 | 0.880 | 0.918 | 0.859 | 0.978 | 0.930 | 0.861 | 1 |
| ISG15, HERC6, RETN, MMP8 | | | | | | | | | |
| RSAD2, IFI27, OAS1, IFIT1, SIGLEC1, | 0.871 | 0.862 | 0.880 | 0.929 | 0.878 | 0.979 | 0.949 | 0.887 | 1 |
| SLC1A2, IL1R2, OLAH, RETN | | | | | | | | | |
| RSAD2, IFI27, IFIT1, SIGLEC1, HERC6, | 0.871 | 0.861 | 0.880 | 0.928 | 0.879 | 0.977 | 0.943 | 0.889 | 0.998 |
| SLC1A2, IL1R2, OLAH, MMP8 | | | | | | | | | |
| RSAD2, IFI27, OAS1, IFIT1, IFI44L, | 0.871 | 0.861 | 0.880 | 0.927 | 0.875 | 0.978 | 0.934 | 0.862 | 1 |
| HERC6, SLC1A2, OLAH, RETN | | | | | | | | | |
| IFI27, OAS1, IFIT1, IFI44L, ISG15, | 0.871 | 0.861 | 0.880 | 0.918 | 0.861 | 0.976 | 0.948 | 0.894 | 1 |
| IL1R2, OLAH, RETN, MMP8 | | | | | | | | | |
| RSAD2, IFI27, OAS1, IFI44L, SIGLEC1, | 0.871 | 0.862 | 0.880 | 0.929 | 0.879 | 0.980 | 0.947 | 0.891 | 1 |
| ISG15, SLC1A2, IL1R2, OLAH | | | | | | | | | |
| RSAD2, IFI27, IFI44L, ISG15, HERC6, | 0.871 | 0.861 | 0.880 | 0.928 | 0.878 | 0.979 | 0.941 | 0.881 | 1 |
| SLC1A2, OLAH, RETN, MMP8 | | | | | | | | | |
| RSAD2, IFIT1, IFI44L, SIGLEC1, ISG15, | 0.871 | 0.860 | 0.881 | 0.917 | 0.859 | 0.975 | 0.949 | 0.891 | 1 |
| HERC6, OLAH, FAM20A, MMP8 | | | | | | | | | |
| IFI27, OAS1, IFIT1, SIGLEC1, ISG15, | 0.871 | 0.860 | 0.881 | 0.929 | 0.878 | 0.981 | 0.954 | 0.897 | 1 |
| SLC1A2, OLAH, RETN, MMP8 | | | | | | | | | |
| RSAD2, IFI27, OAS1, IFIT1, IFI44L, | 0.871 | 0.862 | 0.879 | 0.926 | 0.873 | 0.979 | 0.950 | 0.887 | 1 |
| SIGLEC1, SLC1A2, OLAH, RETN | | | | | | | | | |
| RSAD2, IFI27, IFI44L, SIGLEC1, | 0.871 | 0.861 | 0.880 | 0.930 | 0.881 | 0.980 | 0.959 | 0.910 | 1 |
| SLC1A2, IL1R2, OLAH, RETN, MMP8 | | | | | | | | | |
| IFI27, OAS1, IFI44L, SIGLEC1, ISG15, | 0.871 | 0.861 | 0.881 | 0.929 | 0.881 | 0.978 | 0.939 | 0.882 | 0.996 |
| HERC6, SLC1A2, IL1R2, OLAH | | | | | | | | | |
| RSAD2, IFI27, OAS1, IFI44L, SIGLEC1, | 0.871 | 0.862 | 0.880 | 0.930 | 0.880 | 0.981 | 0.950 | 0.896 | 1 |
| ISG15, SLC1A2, OLAH, MMP8 | | | | | | | | | |
| RSAD2, IFIT1, SIGLEC1, ISG15, | 0.871 | 0.860 | 0.881 | 0.926 | 0.877 | 0.975 | 0.945 | 0.874 | 1 |
| HERC6, SLC1A2, OLAH, FAM20A, MMP8 | | | | | | | | | |
| OAS1, SIGLEC1, ISG15, HERC6, | 0.871 | 0.860 | 0.881 | 0.922 | 0.873 | 0.971 | 0.961 | 0.901 | 1 |
| SLC1A2, IL1R2, OLAH, FAM20A, MMP8 | | | | | | | | | |
| RSAD2, IFI27, OAS1, IFIT1, ISG15, | 0.871 | 0.861 | 0.880 | 0.928 | 0.877 | 0.979 | 0.930 | 0.858 | 1 |
| HERC6, SLC1A2, OLAH, RETN | | | | | | | | | |
| RSAD2, IFI27, IFI44L, SIGLEC1, ISG15, | 0.871 | 0.861 | 0.880 | 0.931 | 0.881 | 0.980 | 0.940 | 0.883 | 0.998 |
| HERC6, SLC1A2, OLAH, MMP8 | | | | | | | | | |
| IFI27, OAS1, IFI44L, HERC6, SLC1A2, | 0.871 | 0.860 | 0.881 | 0.930 | 0.881 | 0.980 | 0.938 | 0.872 | 1 |
| IL1R2, OLAH, RETN, MMP8 | | | | | | | | | |
| RSAD2, IFI27, SIGLEC1, ISG15, | 0.871 | 0.861 | 0.880 | 0.930 | 0.880 | 0.979 | 0.953 | 0.900 | 1 |
| SLC1A2, IL1R2, OLAH, RETN, MMP8 | | | | | | | | | |
| IFI27, OAS1, IFI44L, SIGLEC1, ISG15, | 0.871 | 0.862 | 0.879 | 0.928 | 0.881 | 0.975 | 0.940 | 0.881 | 0.999 |
| HERC6, SLC1A2, IL1R2, RETN | | | | | | | | | |
| RSAD2, IFI27, OAS1, IFI44L, ISG15, | 0.871 | 0.861 | 0.880 | 0.929 | 0.879 | 0.979 | 0.933 | 0.861 | 1 |
| HERC6, SLC1A2, OLAH, RETN | | | | | | | | | |
| IFIT1, IFI44L, SIGLEC1, ISG15, HERC6, | 0.871 | 0.861 | 0.880 | 0.925 | 0.876 | 0.975 | 0.937 | 0.867 | 1 |
| SLC1A2, IL1R2, OLAH, FAM20A | | | | | | | | | |
| RSAD2, IFI27, OAS1, IFI44L, ISG15, | 0.871 | 0.860 | 0.881 | 0.930 | 0.884 | 0.975 | 0.926 | 0.839 | 1 |
| HERC6, SLC1A2, IL1R2, FAM20A | | | | | | | | | |
| IFI27, OAS1, IFIT1, IFI44L, SIGLEC1, | 0.871 | 0.862 | 0.880 | 0.930 | 0.879 | 0.980 | 0.946 | 0.885 | 1 |
| ISG15, SLC1A2, OLAH, RETN | | | | | | | | | |
| IFI27, OAS1, IFIT1, ISG15, HERC6, | 0.871 | 0.860 | 0.881 | 0.930 | 0.884 | 0.976 | 0.927 | 0.842 | 1 |
| SLC1A2, IL1R2, FAM20A, MMP8 | | | | | | | | | |
| IFI27, OAS1, IFIT1, IFI44L, SIGLEC1, | 0.871 | 0.861 | 0.880 | 0.928 | 0.880 | 0.976 | 0.946 | 0.890 | 1 |
| HERC6, SLC1A2, IL1R2, OLAH | | | | | | | | | |
| RSAD2, IFI27, OAS1, IFIT1, SIGLEC1, | 0.871 | 0.861 | 0.880 | 0.923 | 0.872 | 0.974 | 0.945 | 0.894 | 0.995 |
| ISG15, IL1R2, OLAH, MMP8 | | | | | | | | | |
| OAS1, IFIT1, IFI44L, SIGLEC1, ISG15, | 0.871 | 0.861 | 0.880 | 0.919 | 0.862 | 0.976 | 0.955 | 0.900 | 1 |
| HERC6, IL1R2, FAM20A, RETN | | | | | | | | | |
| RSAD2, IFI27, OAS1, IFIT1, SIGLEC1, | 0.871 | 0.861 | 0.880 | 0.926 | 0.875 | 0.976 | 0.951 | 0.897 | 1 |
| SLC1A2, IL1R2, OLAH, MMP8 | | | | | | | | | |
| IFI27, OAS1, IFIT1, IFI44L, SLC1A2, | 0.870 | 0.861 | 0.880 | 0.930 | 0.880 | 0.979 | 0.927 | 0.844 | 1 |
| IL1R2, FAM20A, RETN, MMP8 | | | | | | | | | |
| RSAD2, IFI27, IFI44L, SIGLEC1, ISG15, | 0.870 | 0.861 | 0.880 | 0.928 | 0.878 | 0.978 | 0.943 | 0.890 | 0.997 |
| HERC6, SLC1A2, IL1R2, OLAH | | | | | | | | | |
| RSAD2, IFI27, OAS1, SIGLEC1, | 0.870 | 0.861 | 0.880 | 0.929 | 0.879 | 0.979 | 0.954 | 0.898 | 1 |
| SLC1A2, IL1R2, OLAH, RETN, MMP8 | | | | | | | | | |
| RSAD2, IFI27, IFIT1, SIGLEC1, ISG15, | 0.870 | 0.861 | 0.879 | 0.929 | 0.880 | 0.978 | 0.942 | 0.887 | 0.998 |
| SLC1A2, IL1R2, OLAH, MMP8 | | | | | | | | | |
| IFI27, OAS1, IFIT1, IFI44L, SIGLEC1, | 0.870 | 0.861 | 0.880 | 0.925 | 0.873 | 0.977 | 0.958 | 0.913 | 1 |
| ISG15, IL1R2, RETN, MMP8 | | | | | | | | | |
| IFIT1, IFI44L, SIGLEC1, ISG15, HERC6, | 0.870 | 0.861 | 0.880 | 0.924 | 0.873 | 0.975 | 0.930 | 0.858 | 1 |
| SLC1A2, OLAH, FAM20A, RETN | | | | | | | | | |
| RSAD2, IFI27, OAS1, IFIT1, IFI44L, | 0.870 | 0.862 | 0.879 | 0.930 | 0.880 | 0.981 | 0.948 | 0.892 | 1 |
| SIGLEC1, ISG15, SLC1A2, OLAH | | | | | | | | | |
| RSAD2, IFI27, OAS1, IFIT1, SIGLEC1, | 0.870 | 0.861 | 0.880 | 0.928 | 0.875 | 0.981 | 0.954 | 0.897 | 1 |
| SLC1A2, OLAH, RETN, MMP8 | | | | | | | | | |
| RSAD2, IFI27, OAS1, ISG15, HERC6, | 0.870 | 0.861 | 0.880 | 0.929 | 0.880 | 0.977 | 0.930 | 0.862 | 0.999 |
| SLC1A2, IL1R2, OLAH, MMP8 | | | | | | | | | |
| OAS1, IFI44L, SIGLEC1, ISG15, HERC6, | 0.870 | 0.861 | 0.880 | 0.918 | 0.860 | 0.976 | 0.958 | 0.894 | 1 |
| IL1R2, FAM20A, RETN, MMP8 | | | | | | | | | |
| RSAD2, IFI27, OAS1, IFI44L, SIGLEC1, | 0.870 | 0.862 | 0.879 | 0.929 | 0.877 | 0.981 | 0.948 | 0.885 | 1 |
| ISG15, SLC1A2, OLAH, RETN | | | | | | | | | |
| RSAD2, IFI27, IFIT1, SIGLEC1, ISG15, | 0.870 | 0.861 | 0.880 | 0.929 | 0.880 | 0.979 | 0.936 | 0.857 | 1 |
| HERC6, SLC1A2, FAM20A, MMP8 | | | | | | | | | |
| IFIT1, IFI44L, SIGLEC1, ISG15, | 0.870 | 0.861 | 0.880 | 0.922 | 0.869 | 0.975 | 0.949 | 0.888 | 1 |
| SLC1A2, OLAH, FAM20A, RETN, MMP8 | | | | | | | | | |
| RSAD2, IFI27, OAS1, SIGLEC1, ISG15, | 0.870 | 0.862 | 0.879 | 0.926 | 0.878 | 0.975 | 0.948 | 0.888 | 1 |
| HERC6, SLC1A2, IL1R2, RETN | | | | | | | | | |
| RSAD2, OAS1, IFI44L, SIGLEC1, ISG15, | 0.870 | 0.860 | 0.880 | 0.919 | 0.862 | 0.975 | 0.962 | 0.905 | 1 |
| IL1R2, OLAH, FAM20A, MMP8 | | | | | | | | | |
| OAS1, IFIT1, IFI44L, SIGLEC1, ISG15, | 0.870 | 0.860 | 0.881 | 0.925 | 0.875 | 0.975 | 0.929 | 0.859 | 1 |
| HERC6, SLC1A2, OLAH, FAM20A | | | | | | | | | |
| RSAD2, IFI27, OAS1, IFI44L, SIGLEC1, | 0.870 | 0.861 | 0.879 | 0.925 | 0.874 | 0.977 | 0.949 | 0.886 | 1 |
| SLC1A2, IL1R2, OLAH, RETN | | | | | | | | | |
| IFI27, IFIT1, IFI44L, SIGLEC1, SLC1A2, | 0.870 | 0.861 | 0.880 | 0.930 | 0.880 | 0.980 | 0.958 | 0.908 | 1 |
| IL1R2, OLAH, RETN, MMP8 | | | | | | | | | |
| IFI27, IFIT1, IFI44L, SIGLEC1, ISG15, | 0.870 | 0.861 | 0.879 | 0.929 | 0.879 | 0.979 | 0.946 | 0.887 | 1 |
| SLC1A2, IL1R2, OLAH, RETN | | | | | | | | | |
| RSAD2, IFI27, IFI44L, SIGLEC1, ISG15, | 0.870 | 0.862 | 0.879 | 0.929 | 0.878 | 0.979 | 0.943 | 0.884 | 1 |
| SLC1A2, IL1R2, OLAH, RETN | | | | | | | | | |
| RSAD2, IFI27, OAS1, IFIT1, IFI44L, | 0.870 | 0.861 | 0.880 | 0.928 | 0.880 | 0.976 | 0.926 | 0.840 | 1 |
| SLC1A2, IL1R2, FAM20A, MMP8 | | | | | | | | | |
| IFI27, OAS1, IFI44L, SIGLEC1, ISG15, | 0.870 | 0.861 | 0.880 | 0.929 | 0.878 | 0.980 | 0.952 | 0.895 | 1 |
| SLC1A2, OLAH, RETN, MMP8 | | | | | | | | | |
| RSAD2, IFI27, IFIT1, SIGLEC1, ISG15, | 0.870 | 0.861 | 0.879 | 0.929 | 0.879 | 0.980 | 0.940 | 0.879 | 1 |
| SLC1A2, IL1R2, OLAH, RETN | | | | | | | | | |
| RSAD2, OAS1, IFIT1, IFI44L, SIGLEC1, | 0.870 | 0.860 | 0.880 | 0.926 | 0.876 | 0.976 | 0.933 | 0.863 | 1 |
| HERC6, SLC1A2, OLAH, FAM20A | | | | | | | | | |
| RSAD2, IFI27, OAS1, SIGLEC1, ISG15, | 0.870 | 0.861 | 0.879 | 0.929 | 0.879 | 0.979 | 0.945 | 0.887 | 1 |
| SLC1A2, IL1R2, OLAH, MMP8 | | | | | | | | | |
| IFI27, IFIT1, ISG15, HERC6, SLC1A2, | 0.870 | 0.860 | 0.880 | 0.929 | 0.879 | 0.979 | 0.941 | 0.881 | 1 |
| IL1R2, OLAH, RETN, MMP8 | | | | | | | | | |
| RSAD2, IFI27, IFIT1, IFI44L, SIGLEC1, | 0.870 | 0.861 | 0.879 | 0.926 | 0.876 | 0.977 | 0.941 | 0.885 | 0.997 |
| HERC6, SLC1A2, OLAH, MMP8 | | | | | | | | | |
| RSAD2, IFI27, OAS1, IFIT1, ISG15, | 0.870 | 0.861 | 0.880 | 0.929 | 0.879 | 0.978 | 0.933 | 0.866 | 0.999 |
| HERC6, SLC1A2, IL1R2, OLAH | | | | | | | | | |
| RSAD2, IFIT1, SIGLEC1, ISG15, IL1R2, | 0.870 | 0.861 | 0.879 | 0.920 | 0.863 | 0.978 | 0.967 | 0.916 | 1 |
| OLAH, FAM20A, RETN, MMP8 | | | | | | | | | |
| RSAD2, IFI27, OAS1, IFIT1, ISG15, | 0.870 | 0.860 | 0.880 | 0.921 | 0.865 | 0.977 | 0.950 | 0.897 | 1 |
| IL1R2, OLAH, RETN, MMP8 | | | | | | | | | |
| OAS1, IFIT1, IFI44L, SIGLEC1, ISG15, | 0.870 | 0.859 | 0.881 | 0.917 | 0.858 | 0.975 | 0.948 | 0.890 | 1 |
| HERC6, OLAH, FAM20A, MMP8 | | | | | | | | | |
| IFI27, OAS1, IFIT1, IFI44L, SIGLEC1, | 0.870 | 0.861 | 0.879 | 0.927 | 0.875 | 0.978 | 0.950 | 0.891 | 1 |
| SLC1A2, IL1R2, OLAH, RETN | | | | | | | | | |
| IFI27, OAS1, IFIT1, IFI44L, SIGLEC1, | 0.870 | 0.862 | 0.879 | 0.924 | 0.878 | 0.971 | 0.942 | 0.891 | 0.994 |
| ISG15, HERC6, IL1R2, MMP8 | | | | | | | | | |
| IFI27, OAS1, IFIT1, IFI44L, ISG15, | 0.870 | 0.860 | 0.880 | 0.928 | 0.878 | 0.978 | 0.934 | 0.867 | 1 |
| HERC6, SLC1A2, OLAH, RETN | | | | | | | | | |
| RSAD2, IFI27, OAS1, IFIT1, IFI44L, | 0.870 | 0.861 | 0.879 | 0.922 | 0.868 | 0.976 | 0.959 | 0.911 | 1 |
| SIGLEC1, IL1R2, RETN, MMP8 | | | | | | | | | |
| RSAD2, IFI27, OAS1, ISG15, HERC6, | 0.870 | 0.860 | 0.880 | 0.932 | 0.885 | 0.980 | 0.924 | 0.836 | 1 |
| SLC1A2, IL1R2, FAM20A, RETN | | | | | | | | | |
| RSAD2, IFI27, OAS1, SIGLEC1, ISG15, | 0.870 | 0.859 | 0.881 | 0.925 | 0.869 | 0.982 | 0.942 | 0.868 | 1 |
| SLC1A2, FAM20A, RETN, MMP8 | | | | | | | | | |
| RSAD2, IFI27, IFIT1, IFI44L, SLC1A2, | 0.870 | 0.860 | 0.880 | 0.932 | 0.883 | 0.981 | 0.928 | 0.845 | 1 |
| IL1R2, FAM20A, RETN, MMP8 | | | | | | | | | |
| OAS1, IFIT1, IFI44L, SIGLEC1, ISG15, | 0.870 | 0.860 | 0.880 | 0.916 | 0.858 | 0.975 | 0.940 | 0.880 | 1 |
| HERC6, OLAH, FAM20A, RETN | | | | | | | | | |
| RSAD2, OAS1, IFIT1, SIGLEC1, ISG15, | 0.870 | 0.861 | 0.879 | 0.921 | 0.863 | 0.979 | 0.947 | 0.882 | 1 |
| HERC6, FAM20A, RETN, MMP8 | | | | | | | | | |
| IFI27, IFIT1, ISG15, HERC6, SLC1A2, | 0.870 | 0.860 | 0.880 | 0.934 | 0.886 | 0.983 | 0.930 | 0.847 | 1 |
| IL1R2, FAM20A, RETN, MMP8 | | | | | | | | | |
| IFI27, OAS1, IFIT1, IFI44L, SIGLEC1, | 0.870 | 0.861 | 0.879 | 0.928 | 0.879 | 0.977 | 0.951 | 0.899 | 1 |
| SLC1A2, IL1R2, OLAH, MMP8 | | | | | | | | | |
| RSAD2, IFI27, OAS1, IFIT1, SIGLEC1, | 0.870 | 0.861 | 0.879 | 0.920 | 0.865 | 0.976 | 0.949 | 0.892 | 1 |
| ISG15, IL1R2, OLAH, RETN | | | | | | | | | |
| IFI27, OAS1, IFIT1, IFI44L, HERC6, | 0.870 | 0.860 | 0.880 | 0.927 | 0.876 | 0.978 | 0.934 | 0.867 | 1 |
| SLC1A2, IL1R2, OLAH, RETN | | | | | | | | | |
| IFI27, OAS1, IFIT1, IFI44L, ISG15, | 0.870 | 0.860 | 0.880 | 0.924 | 0.869 | 0.979 | 0.929 | 0.848 | 1 |
| HERC6, SLC1A2, FAM20A, RETN | | | | | | | | | |
| RSAD2, IFI27, IFIT1, SIGLEC1, ISG15, | 0.870 | 0.860 | 0.880 | 0.930 | 0.880 | 0.980 | 0.940 | 0.883 | 0.997 |
| HERC6, SLC1A2, OLAH, MMP8 | | | | | | | | | |
| RSAD2, IFI27, OAS1, IFIT1, IFI44L, | 0.870 | 0.861 | 0.878 | 0.922 | 0.874 | 0.971 | 0.948 | 0.900 | 0.996 |
| SIGLEC1, ISG15, HERC6, IL1R2 | | | | | | | | | |
| RSAD2, IFI27, OAS1, IFIT1, HERC6, | 0.870 | 0.860 | 0.880 | 0.929 | 0.880 | 0.977 | 0.935 | 0.869 | 1 |
| SLC1A2, IL1R2, OLAH, MMP8 | | | | | | | | | |
| RSAD2, IFI27, OAS1, IFI44L, HERC6, | 0.870 | 0.860 | 0.880 | 0.927 | 0.875 | 0.978 | 0.932 | 0.860 | 1 |
| SLC1A2, IL1R2, OLAH, RETN | | | | | | | | | |
| RSAD2, OAS1, IFIT1, SIGLEC1, ISG15, | 0.870 | 0.861 | 0.879 | 0.920 | 0.863 | 0.977 | 0.952 | 0.895 | 1 |
| HERC6, IL1R2, FAM20A, RETN | | | | | | | | | |
| RSAD2, IFI27, OAS1, IFIT1, IFI44L, | 0.870 | 0.861 | 0.879 | 0.926 | 0.874 | 0.978 | 0.954 | 0.902 | 1 |
| SIGLEC1, SLC1A2, OLAH, MMP8 | | | | | | | | | |
| RSAD2, IFI27, OAS1, IFI44L, ISG15, | 0.870 | 0.860 | 0.879 | 0.923 | 0.867 | 0.978 | 0.949 | 0.894 | 1 |
| IL1R2, OLAH, RETN, MMP8 | | | | | | | | | |
| IFI27, OAS1, IFI44L, SIGLEC1, ISG15, | 0.870 | 0.861 | 0.879 | 0.930 | 0.882 | 0.978 | 0.946 | 0.892 | 0.999 |
| SLC1A2, IL1R2, OLAH, MMP8 | | | | | | | | | |
| IFI27, IFIT1, IFI44L, SIGLEC1, ISG15, | 0.870 | 0.861 | 0.879 | 0.929 | 0.881 | 0.978 | 0.941 | 0.886 | 0.997 |
| HERC6, SLC1A2, IL1R2, OLAH | | | | | | | | | |
| RSAD2, IFI27, OAS1, ISG15, HERC6, | 0.870 | 0.860 | 0.880 | 0.931 | 0.886 | 0.976 | 0.921 | 0.829 | 1 |
| SLC1A2, IL1R2, FAM20A, MMP8 | | | | | | | | | |
| RSAD2, IFI27, OAS1, IFIT1, IFI44L, | 0.870 | 0.861 | 0.879 | 0.928 | 0.878 | 0.978 | 0.949 | 0.892 | 1 |
| SIGLEC1, SLC1A2, IL1R2, OLAH | | | | | | | | | |
| RSAD2, IFI27, OAS1, SIGLEC1, ISG15, | 0.870 | 0.860 | 0.879 | 0.928 | 0.876 | 0.981 | 0.953 | 0.894 | 1 |
| SLC1A2, OLAH, RETN, MMP8 | | | | | | | | | |
| IFI27, IFIT1, IFI44L, ISG15, HERC6, | 0.870 | 0.860 | 0.880 | 0.929 | 0.881 | 0.976 | 0.928 | 0.841 | 1 |
| SLC1A2, IL1R2, FAM20A, MMP8 | | | | | | | | | |
| RSAD2, IFI27, IFIT1, IFI44L, ISG15, | 0.870 | 0.860 | 0.879 | 0.919 | 0.861 | 0.976 | 0.949 | 0.895 | 1 |
| IL1R2, OLAH, RETN, MMP8 | | | | | | | | | |
| RSAD2, IFI27, IFIT1, SIGLEC1, ISG15, | 0.870 | 0.860 | 0.879 | 0.928 | 0.876 | 0.980 | 0.953 | 0.898 | 1 |
| SLC1A2, OLAH, RETN, MMP8 | | | | | | | | | |
| RSAD2, OAS1, IFIT1, IFI44L, SIGLEC1, | 0.870 | 0.860 | 0.880 | 0.923 | 0.867 | 0.979 | 0.968 | 0.918 | 1 |
| IL1R2, FAM20A, RETN, MMP8 | | | | | | | | | |
| RSAD2, IFIT1, SIGLEC1, ISG15, | 0.870 | 0.861 | 0.879 | 0.918 | 0.860 | 0.977 | 0.952 | 0.897 | 1 |
| HERC6, IL1R2, OLAH, FAM20A, RETN | | | | | | | | | |
| RSAD2, IFI44L, SIGLEC1, HERC6, | 0.870 | 0.859 | 0.880 | 0.924 | 0.876 | 0.973 | 0.959 | 0.897 | 1 |
| SLC1A2, IL1R2, OLAH, FAM20A, MMP8 | | | | | | | | | |
| RSAD2, IFI27, IFIT1, IFI44L, SIGLEC1, | 0.870 | 0.861 | 0.879 | 0.930 | 0.881 | 0.980 | 0.940 | 0.883 | 0.998 |
| ISG15, HERC6, SLC1A2, OLAH | | | | | | | | | |
| RSAD2, OAS1, IFIT1, SIGLEC1, HERC6, | 0.870 | 0.860 | 0.879 | 0.918 | 0.860 | 0.977 | 0.955 | 0.901 | 1 |
| IL1R2, OLAH, FAM20A, RETN | | | | | | | | | |
| IFI27, OAS1, IFIT1, ISG15, SLC1A2, | 0.870 | 0.860 | 0.880 | 0.931 | 0.882 | 0.980 | 0.927 | 0.844 | 1 |
| IL1R2, FAM20A, RETN, MMP8 | | | | | | | | | |
| RSAD2, IFI27, OAS1, IFI44L, SIGLEC1, | 0.870 | 0.861 | 0.878 | 0.924 | 0.876 | 0.972 | 0.940 | 0.888 | 0.993 |
| ISG15, HERC6, IL1R2, MMP8 | | | | | | | | | |
| RSAD2, IFI27, IFIT1, IFI44L, HERC6, | 0.870 | 0.860 | 0.880 | 0.930 | 0.882 | 0.979 | 0.926 | 0.842 | 1 |
| SLC1A2, IL1R2, FAM20A, RETN | | | | | | | | | |
| OAS1, IFIT1, IFI44L, SIGLEC1, HERC6, | 0.870 | 0.860 | 0.880 | 0.927 | 0.879 | 0.975 | 0.934 | 0.865 | 1 |
| SLC1A2, IL1R2, OLAH, FAM20A | | | | | | | | | |
| RSAD2, IFI27, OAS1, IFIT1, ISG15, | 0.870 | 0.860 | 0.880 | 0.929 | 0.879 | 0.979 | 0.929 | 0.861 | 0.998 |
| HERC6, SLC1A2, OLAH, MMP8 | | | | | | | | | |
| RSAD2, IFI27, IFIT1, IFI44L, SIGLEC1, | 0.870 | 0.860 | 0.879 | 0.929 | 0.877 | 0.981 | 0.958 | 0.905 | 1 |
| SLC1A2, OLAH, RETN, MMP8 | | | | | | | | | |
| RSAD2, OAS1, SIGLEC1, ISG15, IL1R2, | 0.870 | 0.860 | 0.880 | 0.918 | 0.859 | 0.976 | 0.963 | 0.908 | 1 |
| OLAH, FAM20A, RETN, MMP8 | | | | | | | | | |
| RSAD2, IFIT1, SIGLEC1, HERC6, | 0.870 | 0.860 | 0.879 | 0.928 | 0.881 | 0.975 | 0.957 | 0.889 | 1 |
| SLC1A2, IL1R2, FAM20A, RETN, MMP8 | | | | | | | | | |
| OAS1, IFI44L, SIGLEC1, HERC6, | 0.870 | 0.859 | 0.880 | 0.924 | 0.875 | 0.972 | 0.960 | 0.900 | 1 |
| SLC1A2, IL1R2, OLAH, FAM20A, MMP8 | | | | | | | | | |
| RSAD2, OAS1, SIGLEC1, ISG15, | 0.870 | 0.860 | 0.880 | 0.924 | 0.876 | 0.972 | 0.958 | 0.892 | 1 |
| HERC6, SLC1A2, IL1R2, FAM20A, MMP8 | | | | | | | | | |
| RSAD2, IFI27, OAS1, IFIT1, SLC1A2, | 0.870 | 0.860 | 0.879 | 0.932 | 0.883 | 0.981 | 0.929 | 0.848 | 1 |
| IL1R2, FAM20A, RETN, MMP8 | | | | | | | | | |
| IFIT1, IFI44L, SIGLEC1, SLC1A2, | 0.870 | 0.860 | 0.879 | 0.928 | 0.880 | 0.976 | 0.961 | 0.903 | 1 |
| IL1R2, OLAH, FAM20A, RETN, MMP8 | | | | | | | | | |
| RSAD2, OAS1, IFI44L, SIGLEC1, | 0.870 | 0.860 | 0.880 | 0.921 | 0.865 | 0.977 | 0.960 | 0.899 | 1 |
| HERC6, IL1R2, FAM20A, RETN, MMP8 | | | | | | | | | |
| RSAD2, IFI27, IFIT1, ISG15, SLC1A2, | 0.870 | 0.860 | 0.879 | 0.933 | 0.884 | 0.981 | 0.929 | 0.847 | 1 |
| IL1R2, FAM20A, RETN, MMP8 | | | | | | | | | |
| IFI27, OAS1, IFI44L, SIGLEC1, ISG15, | 0.870 | 0.861 | 0.879 | 0.929 | 0.878 | 0.979 | 0.947 | 0.888 | 1 |
| SLC1A2, IL1R2, OLAH, RETN | | | | | | | | | |
| RSAD2, IFIT1, SIGLEC1, HERC6, | 0.870 | 0.859 | 0.880 | 0.928 | 0.879 | 0.977 | 0.947 | 0.879 | 1 |
| SLC1A2, OLAH, FAM20A, RETN, MMP8 | | | | | | | | | |
| RSAD2, OAS1, SIGLEC1, ISG15, | 0.870 | 0.860 | 0.879 | 0.923 | 0.874 | 0.972 | 0.961 | 0.901 | 1 |
| SLC1A2, IL1R2, OLAH, FAM20A, MMP8 | | | | | | | | | |
| IFI27, IFIT1, IFI44L, ISG15, HERC6, | 0.870 | 0.860 | 0.879 | 0.930 | 0.882 | 0.979 | 0.926 | 0.842 | 1 |
| SLC1A2, IL1R2, FAM20A, RETN | | | | | | | | | |
| RSAD2, IFI27, OAS1, IFIT1, IFI44L, | 0.870 | 0.860 | 0.879 | 0.919 | 0.866 | 0.972 | 0.942 | 0.889 | 0.995 |
| ISG15, IL1R2, OLAH, MMP8 | | | | | | | | | |
| IFI27, IFIT1, IFI44L, HERC6, SLC1A2, | 0.869 | 0.860 | 0.879 | 0.926 | 0.874 | 0.978 | 0.941 | 0.880 | 1 |
| IL1R2, OLAH, RETN, MMP8 | | | | | | | | | |
| RSAD2, IFI27, OAS1, IFIT1, SIGLEC1, | 0.869 | 0.861 | 0.878 | 0.930 | 0.879 | 0.982 | 0.946 | 0.882 | 1 |
| ISG15, SLC1A2, OLAH, RETN | | | | | | | | | |
| RSAD2, IFI27, IFI44L, SIGLEC1, ISG15, | 0.869 | 0.859 | 0.880 | 0.927 | 0.875 | 0.980 | 0.943 | 0.870 | 1 |
| HERC6, SLC1A2, FAM20A, RETN | | | | | | | | | |
| RSAD2, IFI27, OAS1, IFI44L, SIGLEC1, | 0.869 | 0.860 | 0.879 | 0.927 | 0.877 | 0.977 | 0.951 | 0.897 | 1 |
| SLC1A2, IL1R2, OLAH, MMP8 | | | | | | | | | |
| RSAD2, IFI27, OAS1, IFI44L, SIGLEC1, | 0.869 | 0.859 | 0.880 | 0.916 | 0.857 | 0.975 | 0.943 | 0.873 | 1 |
| ISG15, HERC6, FAM20A, MMP8 | | | | | | | | | |
| RSAD2, IFI27, OAS1, IFIT1, IFI44L, | 0.869 | 0.860 | 0.879 | 0.919 | 0.859 | 0.978 | 0.950 | 0.893 | 1 |
| ISG15, OLAH, RETN, MMP8 | | | | | | | | | |
| RSAD2, OAS1, IFIT1, IFI44L, SIGLEC1, | 0.869 | 0.860 | 0.879 | 0.919 | 0.861 | 0.976 | 0.940 | 0.876 | 1 |
| ISG15, HERC6, FAM20A, RETN | | | | | | | | | |
| RSAD2, OAS1, IFIT1, IFI44L, SIGLEC1, | 0.869 | 0.860 | 0.879 | 0.915 | 0.855 | 0.976 | 0.942 | 0.882 | 1 |
| HERC6, OLAH, FAM20A, RETN | | | | | | | | | |
| IFI27, OAS1, IFIT1, ISG15, SLC1A2, | 0.869 | 0.860 | 0.879 | 0.927 | 0.876 | 0.978 | 0.942 | 0.880 | 1 |
| IL1R2, OLAH, RETN, MMP8 | | | | | | | | | |
| IFI44L, SIGLEC1, ISG15, SLC1A2, | 0.869 | 0.860 | 0.879 | 0.923 | 0.873 | 0.972 | 0.957 | 0.890 | 1 |
| IL1R2, OLAH, FAM20A, RETN, MMP8 | | | | | | | | | |
| RSAD2, IFI27, OAS1, IFIT1, IFI44L, | 0.869 | 0.860 | 0.879 | 0.928 | 0.878 | 0.978 | 0.935 | 0.868 | 1 |
| HERC6, SLC1A2, OLAH, MMP8 | | | | | | | | | |
| RSAD2, IFI27, OAS1, IFI44L, SIGLEC1, | 0.869 | 0.861 | 0.878 | 0.922 | 0.867 | 0.977 | 0.940 | 0.873 | 1 |
| ISG15, HERC6, SLC1A2, RETN | | | | | | | | | |
| RSAD2, IFIT1, IFI44L, SIGLEC1, ISG15, | 0.869 | 0.860 | 0.879 | 0.921 | 0.864 | 0.979 | 0.945 | 0.878 | 1 |
| HERC6, FAM20A, RETN, MMP8 | | | | | | | | | |
| RSAD2, IFI27, IFIT1, ISG15, HERC6, | 0.869 | 0.860 | 0.879 | 0.927 | 0.876 | 0.978 | 0.934 | 0.869 | 0.999 |
| SLC1A2, IL1R2, OLAH, RETN | | | | | | | | | |
| IFI27, OAS1, IFI44L, ISG15, HERC6, | 0.869 | 0.859 | 0.879 | 0.929 | 0.878 | 0.979 | 0.932 | 0.864 | 0.999 |
| SLC1A2, IL1R2, OLAH, RETN | | | | | | | | | |
| RSAD2, IFI27, OAS1, IFIT1, IFI44L, | 0.869 | 0.861 | 0.878 | 0.918 | 0.863 | 0.974 | 0.954 | 0.903 | 1 |
| SIGLEC1, ISG15, IL1R2, RETN | | | | | | | | | |
| IFI27, OAS1, IFI44L, SIGLEC1, SLC1A2, | 0.869 | 0.859 | 0.879 | 0.929 | 0.879 | 0.979 | 0.952 | 0.896 | 1 |
| IL1R2, OLAH, RETN, MMP8 | | | | | | | | | |
| OAS1, IFIT1, IFI44L, SIGLEC1, HERC6, | 0.869 | 0.860 | 0.879 | 0.925 | 0.874 | 0.976 | 0.940 | 0.868 | 1 |
| SLC1A2, FAM20A, RETN, MMP8 | | | | | | | | | |
| RSAD2, IFI27, IFIT1, IFI44L, ISG15, | 0.869 | 0.859 | 0.879 | 0.928 | 0.880 | 0.976 | 0.926 | 0.840 | 1 |
| SLC1A2, IL1R2, FAM20A, MMP8 | | | | | | | | | |
| RSAD2, IFI27, IFI44L, ISG15, HERC6, | 0.869 | 0.858 | 0.880 | 0.930 | 0.885 | 0.976 | 0.927 | 0.840 | 1 |
| SLC1A2, IL1R2, FAM20A, MMP8 | | | | | | | | | |
| IFIT1, SIGLEC1, ISG15, SLC1A2, IL1R2, | 0.869 | 0.860 | 0.879 | 0.928 | 0.881 | 0.975 | 0.962 | 0.906 | 1 |
| OLAH, FAM20A, RETN, MMP8 | | | | | | | | | |
| IFI27, OAS1, IFI44L, SIGLEC1, ISG15, | 0.869 | 0.860 | 0.878 | 0.923 | 0.870 | 0.977 | 0.940 | 0.877 | 1 |
| HERC6, SLC1A2, RETN, MMP8 | | | | | | | | | |
| RSAD2, IFI27, OAS1, IFIT1, ISG15, | 0.869 | 0.859 | 0.879 | 0.932 | 0.887 | 0.977 | 0.925 | 0.839 | 1 |
| HERC6, SLC1A2, IL1R2, FAM20A | | | | | | | | | |
| RSAD2, IFI27, IFIT1, IFI44L, ISG15, | 0.869 | 0.860 | 0.879 | 0.928 | 0.880 | 0.976 | 0.925 | 0.838 | 1 |
| HERC6, SLC1A2, IL1R2, FAM20A | | | | | | | | | |
| RSAD2, IFI27, IFIT1, IFI44L, SIGLEC1, | 0.869 | 0.860 | 0.878 | 0.927 | 0.878 | 0.977 | 0.946 | 0.893 | 0.998 |
| HERC6, SLC1A2, IL1R2, OLAH | | | | | | | | | |
| RSAD2, IFI27, IFI44L, ISG15, HERC6, | 0.869 | 0.860 | 0.878 | 0.926 | 0.876 | 0.977 | 0.935 | 0.870 | 0.999 |
| SLC1A2, IL1R2, OLAH, RETN | | | | | | | | | |
| IFI27, OAS1, IFIT1, IFI44L, SIGLEC1, | 0.869 | 0.860 | 0.878 | 0.927 | 0.877 | 0.977 | 0.942 | 0.885 | 1.000 |
| ISG15, SLC1A2, IL1R2, OLAH | | | | | | | | | |
| RSAD2, IFI27, IFIT1, ISG15, HERC6, | 0.869 | 0.859 | 0.879 | 0.929 | 0.882 | 0.976 | 0.929 | 0.845 | 1 |
| SLC1A2, IL1R2, FAM20A, MMP8 | | | | | | | | | |
| RSAD2, IFI27, OAS1, IFI44L, SIGLEC1, | 0.869 | 0.860 | 0.879 | 0.927 | 0.873 | 0.980 | 0.957 | 0.899 | 1 |
| SLC1A2, OLAH, RETN, MMP8 | | | | | | | | | |
| RSAD2, OAS1, IFIT1, SIGLEC1, ISG15, | 0.869 | 0.859 | 0.879 | 0.922 | 0.868 | 0.975 | 0.952 | 0.891 | 1 |
| SLC1A2, OLAH, FAM20A, MMP8 | | | | | | | | | |
| RSAD2, IFI27, IFIT1, ISG15, HERC6, | 0.869 | 0.860 | 0.878 | 0.928 | 0.879 | 0.977 | 0.928 | 0.862 | 0.994 |
| SLC1A2, IL1R2, OLAH, MMP8 | | | | | | | | | |
| RSAD2, IFI27, OAS1, IFIT1, SLC1A2, | 0.869 | 0.860 | 0.879 | 0.925 | 0.873 | 0.978 | 0.942 | 0.880 | 1 |
| IL1R2, OLAH, RETN, MMP8 | | | | | | | | | |
| RSAD2, OAS1, IFIT1, IFI44L, SIGLEC1, | 0.869 | 0.859 | 0.879 | 0.915 | 0.856 | 0.974 | 0.946 | 0.888 | 1 |
| ISG15, HERC6, OLAH, FAM20A | | | | | | | | | |
| RSAD2, OAS1, IFIT1, IFI44L, SIGLEC1, | 0.869 | 0.860 | 0.878 | 0.912 | 0.849 | 0.975 | 0.963 | 0.914 | 1 |
| ISG15, IL1R2, OLAH, FAM20A | | | | | | | | | |
| RSAD2, IFI27, IFIT1, IFI44L, SIGLEC1, | 0.869 | 0.860 | 0.878 | 0.926 | 0.874 | 0.977 | 0.950 | 0.894 | 1 |
| SLC1A2, IL1R2, OLAH, RETN | | | | | | | | | |
| RSAD2, IFI27, IFI44L, SIGLEC1, ISG15, | 0.869 | 0.859 | 0.879 | 0.930 | 0.879 | 0.981 | 0.952 | 0.897 | 1 |
| SLC1A2, OLAH, RETN, MMP8 | | | | | | | | | |
| RSAD2, IFI27, IFIT1, IFI44L, SIGLEC1, | 0.869 | 0.860 | 0.878 | 0.916 | 0.858 | 0.975 | 0.943 | 0.891 | 0.996 |
| ISG15, HERC6, RETN, MMP8 | | | | | | | | | |
| IFI27, OAS1, IFIT1, IFI44L, SIGLEC1, | 0.869 | 0.859 | 0.879 | 0.921 | 0.865 | 0.978 | 0.939 | 0.866 | 1 |
| ISG15, SLC1A2, FAM20A, MMP8 | | | | | | | | | |
| RSAD2, IFI27, IFIT1, IFI44L, SIGLEC1, | 0.869 | 0.861 | 0.877 | 0.929 | 0.878 | 0.980 | 0.946 | 0.887 | 1 |
| ISG15, SLC1A2, OLAH, RETN | | | | | | | | | |
| RSAD2, IFIT1, IFI44L, SIGLEC1, IL1R2, | 0.869 | 0.859 | 0.879 | 0.920 | 0.863 | 0.977 | 0.968 | 0.918 | 1 |
| OLAH, FAM20A, RETN, MMP8 | | | | | | | | | |
| RSAD2, IFI27, IFIT1, IFI44L, SIGLEC1, | 0.869 | 0.860 | 0.878 | 0.931 | 0.882 | 0.981 | 0.941 | 0.883 | 1.000 |
| ISG15, SLC1A2, OLAH, MMP8 | | | | | | | | | |
| RSAD2, OAS1, IFIT1, IFI44L, SIGLEC1, | 0.869 | 0.859 | 0.879 | 0.919 | 0.860 | 0.979 | 0.945 | 0.879 | 1 |
| HERC6, FAM20A, RETN, MMP8 | | | | | | | | | |
| RSAD2, IFI27, IFIT1, SIGLEC1, HERC6, | 0.869 | 0.860 | 0.878 | 0.926 | 0.879 | 0.974 | 0.962 | 0.921 | 1 |
| SLC1A2, IL1R2, RETN, MMP8 | | | | | | | | | |
| OAS1, IFI44L, SIGLEC1, HERC6, IL1R2, | 0.869 | 0.859 | 0.879 | 0.920 | 0.864 | 0.976 | 0.962 | 0.905 | 1 |
| OLAH, FAM20A, RETN, MMP8 | | | | | | | | | |
| RSAD2, OAS1, IFIT1, IFI44L, SIGLEC1, | 0.869 | 0.858 | 0.880 | 0.912 | 0.848 | 0.976 | 0.951 | 0.898 | 1 |
| ISG15, OLAH, FAM20A, MMP8 | | | | | | | | | |
| RSAD2, OAS1, IFIT1, IFI44L, SIGLEC1, | 0.869 | 0.859 | 0.879 | 0.927 | 0.880 | 0.974 | 0.963 | 0.904 | 1 |
| SLC1A2, IL1R2, FAM20A, MMP8 | | | | | | | | | |
| OAS1, IFIT1, IFI44L, SIGLEC1, | 0.869 | 0.859 | 0.879 | 0.924 | 0.872 | 0.977 | 0.948 | 0.888 | 1 |
| SLC1A2, OLAH, FAM20A, RETN, MMP8 | | | | | | | | | |
| RSAD2, IFI27, IFIT1, IFI44L, SIGLEC1, | 0.869 | 0.860 | 0.878 | 0.926 | 0.877 | 0.976 | 0.950 | 0.899 | 1 |
| SLC1A2, IL1R2, OLAH, MMP8 | | | | | | | | | |
| RSAD2, IFI27, IFIT1, ISG15, HERC6, | 0.869 | 0.859 | 0.879 | 0.933 | 0.886 | 0.981 | 0.927 | 0.845 | 1 |
| SLC1A2, IL1R2, FAM20A, RETN | | | | | | | | | |
| RSAD2, IFIT1, IFI44L, SIGLEC1, ISG15, | 0.869 | 0.859 | 0.879 | 0.925 | 0.875 | 0.975 | 0.927 | 0.853 | 1 |
| HERC6, SLC1A2, OLAH, FAM20A | | | | | | | | | |
| OAS1, IFIT1, IFI44L, SIGLEC1, HERC6, | 0.869 | 0.859 | 0.879 | 0.925 | 0.875 | 0.975 | 0.928 | 0.856 | 1 |
| SLC1A2, OLAH, FAM20A, RETN | | | | | | | | | |
| RSAD2, OAS1, IFIT1, IFI44L, SIGLEC1, | 0.869 | 0.859 | 0.878 | 0.920 | 0.863 | 0.977 | 0.953 | 0.898 | 1 |
| HERC6, IL1R2, FAM20A, RETN | | | | | | | | | |
| RSAD2, IFI27, OAS1, IFIT1, HERC6, | 0.869 | 0.859 | 0.879 | 0.931 | 0.883 | 0.979 | 0.924 | 0.837 | 1 |
| SLC1A2, IL1R2, FAM20A, RETN | | | | | | | | | |
| RSAD2, IFI27, IFIT1, IFI44L, ISG15, | 0.869 | 0.860 | 0.878 | 0.926 | 0.875 | 0.976 | 0.934 | 0.869 | 0.999 |
| HERC6, SLC1A2, OLAH, RETN | | | | | | | | | |
| RSAD2, IFIT1, IFI44L, SIGLEC1, | 0.869 | 0.859 | 0.879 | 0.926 | 0.877 | 0.975 | 0.937 | 0.867 | 1 |
| HERC6, SLC1A2, IL1R2, OLAH, FAM20A | | | | | | | | | |
| IFIT1, IFI44L, SIGLEC1, ISG15, | 0.869 | 0.859 | 0.878 | 0.928 | 0.882 | 0.974 | 0.959 | 0.893 | 1 |
| SLC1A2, IL1R2, FAM20A, RETN, MMP8 | | | | | | | | | |
| RSAD2, OAS1, IFI44L, SIGLEC1, | 0.869 | 0.859 | 0.879 | 0.924 | 0.877 | 0.972 | 0.959 | 0.895 | 1 |
| HERC6, SLC1A2, IL1R2, FAM20A, MMP8 | | | | | | | | | |
| OAS1, SIGLEC1, ISG15, HERC6, | 0.869 | 0.859 | 0.879 | 0.926 | 0.878 | 0.974 | 0.959 | 0.895 | 1 |
| SLC1A2, IL1R2, FAM20A, RETN, MMP8 | | | | | | | | | |
| OAS1, IFIT1, IFI44L, SIGLEC1, | 0.869 | 0.859 | 0.879 | 0.927 | 0.881 | 0.974 | 0.963 | 0.906 | 1 |
| SLC1A2, IL1R2, FAM20A, RETN, MMP8 | | | | | | | | | |
| IFI27, IFIT1, IFI44L, ISG15, HERC6, | 0.869 | 0.859 | 0.879 | 0.926 | 0.874 | 0.979 | 0.939 | 0.878 | 1 |
| SLC1A2, OLAH, RETN, MMP8 | | | | | | | | | |
| RSAD2, IFI27, OAS1, IFIT1, IFI44L, | 0.869 | 0.859 | 0.879 | 0.921 | 0.863 | 0.978 | 0.939 | 0.864 | 1 |
| SIGLEC1, ISG15, SLC1A2, FAM20A | | | | | | | | | |
| RSAD2, IFI27, OAS1, IFI44L, HERC6, | 0.869 | 0.859 | 0.879 | 0.932 | 0.884 | 0.979 | 0.925 | 0.838 | 1 |
| SLC1A2, IL1R2, FAM20A, RETN | | | | | | | | | |
| RSAD2, IFI27, IFIT1, IFI44L, HERC6, | 0.869 | 0.859 | 0.878 | 0.926 | 0.874 | 0.977 | 0.935 | 0.870 | 0.999 |
| SLC1A2, IL1R2, OLAH, RETN | | | | | | | | | |
| OAS1, IFIT1, SIGLEC1, SLC1A2, IL1R2, | 0.869 | 0.859 | 0.879 | 0.927 | 0.878 | 0.975 | 0.961 | 0.903 | 1 |
| OLAH, FAM20A, RETN, MMP8 | | | | | | | | | |
| RSAD2, IFI27, OAS1, SIGLEC1, ISG15, | 0.869 | 0.859 | 0.878 | 0.922 | 0.866 | 0.977 | 0.948 | 0.885 | 1 |
| HERC6, SLC1A2, RETN, MMP8 | | | | | | | | | |
| OAS1, IFIT1, SIGLEC1, ISG15, HERC6, | 0.869 | 0.859 | 0.879 | 0.924 | 0.875 | 0.973 | 0.943 | 0.875 | 1 |
| SLC1A2, IL1R2, OLAH, FAM20A | | | | | | | | | |
| RSAD2, OAS1, IFI44L, SIGLEC1, | 0.869 | 0.859 | 0.879 | 0.924 | 0.875 | 0.973 | 0.958 | 0.897 | 1 |
| SLC1A2, IL1R2, OLAH, FAM20A, MMP8 | | | | | | | | | |
| OAS1, SIGLEC1, ISG15, SLC1A2, | 0.869 | 0.859 | 0.879 | 0.923 | 0.873 | 0.972 | 0.961 | 0.903 | 1 |
| IL1R2, OLAH, FAM20A, RETN, MMP8 | | | | | | | | | |
| IFI27, IFIT1, IFI44L, SIGLEC1, HERC6, | 0.869 | 0.859 | 0.878 | 0.927 | 0.878 | 0.976 | 0.952 | 0.904 | 1 |
| SLC1A2, IL1R2, OLAH, MMP8 | | | | | | | | | |
| OAS1, IFIT1, IFI44L, SIGLEC1, ISG15, | 0.869 | 0.858 | 0.879 | 0.926 | 0.880 | 0.973 | 0.961 | 0.897 | 1 |
| SLC1A2, IL1R2, FAM20A, MMP8 | | | | | | | | | |
| RSAD2, IFIT1, IFI44L, SIGLEC1, | 0.869 | 0.859 | 0.878 | 0.918 | 0.860 | 0.976 | 0.952 | 0.895 | 1 |
| HERC6, IL1R2, OLAH, FAM20A, RETN | | | | | | | | | |
| IFI27, IFI44L, SIGLEC1, ISG15, HERC6, | 0.869 | 0.858 | 0.880 | 0.927 | 0.872 | 0.981 | 0.950 | 0.876 | 1 |
| SLC1A2, FAM20A, RETN, MMP8 | | | | | | | | | |
| RSAD2, IFIT1, SIGLEC1, ISG15, | 0.869 | 0.859 | 0.878 | 0.924 | 0.874 | 0.974 | 0.945 | 0.875 | 1 |
| HERC6, SLC1A2, IL1R2, OLAH, FAM20A | | | | | | | | | |
| IFI27, IFIT1, IFI44L, SIGLEC1, HERC6, | 0.869 | 0.860 | 0.878 | 0.927 | 0.878 | 0.975 | 0.955 | 0.909 | 1 |
| SLC1A2, IL1R2, RETN, MMP8 | | | | | | | | | |
| RSAD2, IFI27, OAS1, IFIT1, IFI44L, | 0.869 | 0.859 | 0.878 | 0.917 | 0.860 | 0.974 | 0.940 | 0.880 | 1 |
| ISG15, IL1R2, OLAH, RETN | | | | | | | | | |
| SIGLEC1, ISG15, HERC6, SLC1A2, | 0.869 | 0.859 | 0.879 | 0.923 | 0.873 | 0.972 | 0.957 | 0.890 | 1 |
| IL1R2, OLAH, FAM20A, RETN, MMP8 | | | | | | | | | |
| IFI27, OAS1, IFIT1, IFI44L, ISG15, | 0.869 | 0.858 | 0.879 | 0.928 | 0.878 | 0.978 | 0.928 | 0.862 | 0.995 |
| HERC6, SLC1A2, OLAH, MMP8 | | | | | | | | | |
| IFI27, OAS1, IFI44L, ISG15, HERC6, | 0.868 | 0.858 | 0.878 | 0.927 | 0.878 | 0.976 | 0.928 | 0.861 | 0.995 |
| SLC1A2, IL1R2, OLAH, MMP8 | | | | | | | | | |
| RSAD2, SIGLEC1, HERC6, SLC1A2, | 0.868 | 0.858 | 0.878 | 0.926 | 0.878 | 0.974 | 0.958 | 0.895 | 1 |
| IL1R2, OLAH, FAM20A, RETN, MMP8 | | | | | | | | | |
| RSAD2, IFI27, OAS1, IFI44L, HERC6, | 0.868 | 0.858 | 0.878 | 0.929 | 0.880 | 0.977 | 0.935 | 0.869 | 1 |
| SLC1A2, IL1R2, OLAH, MMP8 | | | | | | | | | |
| RSAD2, IFI27, IFIT1, IFI44L, SIGLEC1, | 0.868 | 0.859 | 0.877 | 0.923 | 0.870 | 0.976 | 0.958 | 0.914 | 1 |
| ISG15, IL1R2, RETN, MMP8 | | | | | | | | | |
| IFI27, OAS1, IFIT1, IFI44L, SIGLEC1, | 0.868 | 0.859 | 0.877 | 0.926 | 0.878 | 0.974 | 0.958 | 0.908 | 1 |
| SLC1A2, IL1R2, RETN, MMP8 | | | | | | | | | |
| RSAD2, IFI27, IFIT1, IFI44L, HERC6, | 0.868 | 0.859 | 0.878 | 0.926 | 0.876 | 0.975 | 0.933 | 0.870 | 0.995 |
| SLC1A2, IL1R2, OLAH, MMP8 | | | | | | | | | |
| RSAD2, IFI27, OAS1, IFI44L, ISG15, | 0.868 | 0.858 | 0.878 | 0.930 | 0.884 | 0.977 | 0.927 | 0.840 | 1 |
| SLC1A2, IL1R2, FAM20A, MMP8 | | | | | | | | | |
| RSAD2, IFI44L, SIGLEC1, HERC6, | 0.868 | 0.858 | 0.878 | 0.920 | 0.864 | 0.976 | 0.961 | 0.903 | 1 |
| IL1R2, OLAH, FAM20A, RETN, MMP8 | | | | | | | | | |
| OAS1, IFI44L, SIGLEC1, ISG15, IL1R2, | 0.868 | 0.858 | 0.878 | 0.915 | 0.856 | 0.975 | 0.964 | 0.909 | 1 |
| OLAH, FAM20A, RETN, MMP8 | | | | | | | | | |
| IFI27, IFIT1, IFI44L, ISG15, SLC1A2, | 0.868 | 0.858 | 0.878 | 0.926 | 0.874 | 0.978 | 0.942 | 0.883 | 1 |
| IL1R2, OLAH, RETN, MMP8 | | | | | | | | | |
| RSAD2, IFI27, IFIT1, IFI44L, SIGLEC1, | 0.868 | 0.858 | 0.878 | 0.922 | 0.866 | 0.977 | 0.938 | 0.863 | 1 |
| ISG15, SLC1A2, FAM20A, MMP8 | | | | | | | | | |
| RSAD2, IFI27, OAS1, IFIT1, IFI44L, | 0.868 | 0.858 | 0.878 | 0.923 | 0.866 | 0.979 | 0.939 | 0.865 | 1 |
| SIGLEC1, SLC1A2, FAM20A, MMP8 | | | | | | | | | |
| RSAD2, IFI44L, SIGLEC1, ISG15, | 0.868 | 0.858 | 0.878 | 0.922 | 0.873 | 0.972 | 0.959 | 0.897 | 1 |
| SLC1A2, IL1R2, OLAH, FAM20A, MMP8 | | | | | | | | | |
| RSAD2, IFI27, OAS1, IFI44L, ISG15, | 0.868 | 0.858 | 0.878 | 0.929 | 0.881 | 0.977 | 0.927 | 0.857 | 0.997 |
| HERC6, SLC1A2, IL1R2, OLAH | | | | | | | | | |
| RSAD2, OAS1, IFIT1, SIGLEC1, ISG15, | 0.868 | 0.858 | 0.878 | 0.927 | 0.881 | 0.974 | 0.959 | 0.893 | 1 |
| SLC1A2, IL1R2, FAM20A, MMP8 | | | | | | | | | |
| IFI27, OAS1, IFIT1, IFI44L, HERC6, | 0.868 | 0.858 | 0.878 | 0.922 | 0.865 | 0.979 | 0.936 | 0.854 | 1 |
| SLC1A2, FAM20A, RETN, MMP8 | | | | | | | | | |
| RSAD2, OAS1, SIGLEC1, HERC6, | 0.868 | 0.858 | 0.878 | 0.926 | 0.878 | 0.974 | 0.957 | 0.891 | 1 |
| SLC1A2, IL1R2, FAM20A, RETN, MMP8 | | | | | | | | | |
| RSAD2, SIGLEC1, ISG15, SLC1A2, | 0.868 | 0.858 | 0.878 | 0.924 | 0.876 | 0.973 | 0.959 | 0.897 | 1 |
| IL1R2, OLAH, FAM20A, RETN, MMP8 | | | | | | | | | |
| RSAD2, IFI27, OAS1, IFI44L, SIGLEC1, | 0.868 | 0.857 | 0.878 | 0.923 | 0.867 | 0.980 | 0.940 | 0.864 | 1 |
| ISG15, SLC1A2, FAM20A, RETN | | | | | | | | | |
| RSAD2, IFI27, IFI44L, ISG15, HERC6, | 0.868 | 0.859 | 0.877 | 0.929 | 0.881 | 0.978 | 0.927 | 0.859 | 0.995 |
| SLC1A2, IL1R2, OLAH, MMP8 | | | | | | | | | |
| RSAD2, IFI27, OAS1, IFIT1, ISG15, | 0.868 | 0.858 | 0.877 | 0.927 | 0.876 | 0.979 | 0.936 | 0.867 | 1 |
| SLC1A2, IL1R2, OLAH, RETN | | | | | | | | | |
| IFI27, IFI44L, SIGLEC1, ISG15, HERC6, | 0.868 | 0.859 | 0.876 | 0.928 | 0.880 | 0.975 | 0.954 | 0.908 | 1 |
| SLC1A2, IL1R2, RETN, MMP8 | | | | | | | | | |
| RSAD2, IFI27, IFIT1, IFI44L, SIGLEC1, | 0.868 | 0.859 | 0.876 | 0.920 | 0.871 | 0.970 | 0.939 | 0.886 | 0.992 |
| ISG15, HERC6, IL1R2, MMP8 | | | | | | | | | |
| RSAD2, IFI27, OAS1, IFI44L, SIGLEC1, | 0.868 | 0.858 | 0.877 | 0.920 | 0.866 | 0.975 | 0.960 | 0.913 | 1 |
| ISG15, IL1R2, RETN, MMP8 | | | | | | | | | |
| RSAD2, IFIT1, IFI44L, SIGLEC1, ISG15, | 0.868 | 0.858 | 0.877 | 0.926 | 0.878 | 0.974 | 0.941 | 0.869 | 1 |
| HERC6, SLC1A2, IL1R2, FAM20A | | | | | | | | | |
| RSAD2, IFI27, OAS1, IFIT1, IFI44L, | 0.868 | 0.858 | 0.877 | 0.926 | 0.877 | 0.975 | 0.937 | 0.873 | 1 |
| HERC6, SLC1A2, IL1R2, OLAH | | | | | | | | | |
| RSAD2, IFI27, OAS1, IFIT1, IFI44L, | 0.868 | 0.858 | 0.877 | 0.923 | 0.867 | 0.978 | 0.941 | 0.877 | 1 |
| SLC1A2, OLAH, RETN, MMP8 | | | | | | | | | |
| RSAD2, IFI27, OAS1, IFIT1, IFI44L, | 0.868 | 0.858 | 0.878 | 0.922 | 0.865 | 0.978 | 0.934 | 0.852 | 1 |
| HERC6, SLC1A2, FAM20A, RETN | | | | | | | | | |
| RSAD2, OAS1, SIGLEC1, SLC1A2, | 0.868 | 0.858 | 0.878 | 0.924 | 0.875 | 0.973 | 0.961 | 0.903 | 1 |
| IL1R2, OLAH, FAM20A, RETN, MMP8 | | | | | | | | | |
| RSAD2, IFI27, IFI44L, SIGLEC1, ISG15, | 0.868 | 0.857 | 0.879 | 0.926 | 0.870 | 0.982 | 0.943 | 0.868 | 1 |
| SLC1A2, FAM20A, RETN, MMP8 | | | | | | | | | |
| RSAD2, IFI27, OAS1, ISG15, SLC1A2, | 0.868 | 0.858 | 0.877 | 0.929 | 0.877 | 0.980 | 0.937 | 0.870 | 1 |
| IL1R2, OLAH, RETN, MMP8 | | | | | | | | | |
| RSAD2, IFI27, OAS1, IFIT1, IFI44L, | 0.868 | 0.858 | 0.877 | 0.925 | 0.872 | 0.978 | 0.933 | 0.865 | 1.000 |
| ISG15, SLC1A2, OLAH, MMP8 | | | | | | | | | |
| RSAD2, IFI27, OAS1, IFIT1, SIGLEC1, | 0.868 | 0.859 | 0.876 | 0.922 | 0.875 | 0.969 | 0.950 | 0.903 | 0.997 |
| ISG15, HERC6, IL1R2, MMP8 | | | | | | | | | |
| RSAD2, IFI27, OAS1, IFI44L, SIGLEC1, | 0.868 | 0.857 | 0.878 | 0.924 | 0.867 | 0.982 | 0.946 | 0.874 | 1 |
| SLC1A2, FAM20A, RETN, MMP8 | | | | | | | | | |
| RSAD2, IFI27, IFIT1, IFI44L, ISG15, | 0.868 | 0.858 | 0.877 | 0.922 | 0.868 | 0.975 | 0.942 | 0.885 | 1.000 |
| HERC6, IL1R2, RETN, MMP8 | | | | | | | | | |
| RSAD2, OAS1, IFIT1, SIGLEC1, ISG15, | 0.868 | 0.857 | 0.878 | 0.922 | 0.870 | 0.973 | 0.940 | 0.874 | 1 |
| HERC6, SLC1A2, OLAH, FAM20A | | | | | | | | | |
| RSAD2, IFI27, OAS1, IFIT1, IFI44L, | 0.868 | 0.858 | 0.877 | 0.928 | 0.877 | 0.978 | 0.930 | 0.863 | 0.998 |
| ISG15, HERC6, SLC1A2, OLAH | | | | | | | | | |
| RSAD2, IFI27, IFIT1, IFI44L, ISG15, | 0.868 | 0.858 | 0.877 | 0.927 | 0.877 | 0.978 | 0.929 | 0.865 | 0.994 |
| HERC6, SLC1A2, OLAH, MMP8 | | | | | | | | | |
| RSAD2, IFI44L, SIGLEC1, ISG15, | 0.867 | 0.857 | 0.878 | 0.926 | 0.879 | 0.973 | 0.959 | 0.893 | 1 |
| HERC6, SLC1A2, IL1R2, FAM20A, MMP8 | | | | | | | | | |
| RSAD2, IFIT1, IFI44L, SIGLEC1, ISG15, | 0.867 | 0.858 | 0.877 | 0.918 | 0.859 | 0.977 | 0.942 | 0.882 | 1 |
| HERC6, OLAH, FAM20A, RETN | | | | | | | | | |
| IFIT1, IFI44L, SIGLEC1, ISG15, HERC6, | 0.867 | 0.858 | 0.877 | 0.925 | 0.877 | 0.974 | 0.941 | 0.869 | 1 |
| SLC1A2, IL1R2, FAM20A, RETN | | | | | | | | | |
| IFI27, OAS1, IFIT1, IFI44L, ISG15, | 0.867 | 0.858 | 0.877 | 0.924 | 0.873 | 0.976 | 0.924 | 0.837 | 1 |
| SLC1A2, IL1R2, FAM20A, RETN | | | | | | | | | |
| RSAD2, IFI27, IFIT1, IFI44L, SLC1A2, | 0.867 | 0.858 | 0.877 | 0.924 | 0.872 | 0.977 | 0.946 | 0.888 | 1 |
| IL1R2, OLAH, RETN, MMP8 | | | | | | | | | |
| IFIT1, IFI44L, SIGLEC1, HERC6, | 0.867 | 0.857 | 0.877 | 0.925 | 0.876 | 0.975 | 0.938 | 0.868 | 1 |
| SLC1A2, IL1R2, OLAH, FAM20A, RETN | | | | | | | | | |
| RSAD2, IFI27, OAS1, IFIT1, ISG15, | 0.867 | 0.857 | 0.877 | 0.927 | 0.878 | 0.975 | 0.926 | 0.841 | 1 |
| SLC1A2, IL1R2, FAM20A, MMP8 | | | | | | | | | |
| OAS1, IFIT1, IFI44L, SIGLEC1, ISG15, | 0.867 | 0.858 | 0.877 | 0.910 | 0.847 | 0.973 | 0.961 | 0.910 | 1 |
| IL1R2, OLAH, FAM20A, RETN | | | | | | | | | |
| RSAD2, IFI27, OAS1, IFIT1, ISG15, | 0.867 | 0.857 | 0.877 | 0.925 | 0.870 | 0.979 | 0.940 | 0.875 | 1 |
| SLC1A2, OLAH, RETN, MMP8 | | | | | | | | | |
| RSAD2, IFI27, IFIT1, IFI44L, SIGLEC1, | 0.867 | 0.859 | 0.876 | 0.927 | 0.878 | 0.976 | 0.946 | 0.895 | 0.997 |
| HERC6, SLC1A2, IL1R2, RETN | | | | | | | | | |
| RSAD2, IFI27, IFI44L, SIGLEC1, | 0.867 | 0.858 | 0.876 | 0.926 | 0.878 | 0.974 | 0.955 | 0.909 | 1 |
| HERC6, SLC1A2, IL1R2, RETN, MMP8 | | | | | | | | | |
| RSAD2, IFI27, OAS1, IFI44L, SLC1A2, | 0.867 | 0.857 | 0.877 | 0.930 | 0.881 | 0.979 | 0.923 | 0.835 | 1 |
| IL1R2, FAM20A, RETN, MMP8 | | | | | | | | | |
| IFI27, IFIT1, IFI44L, ISG15, HERC6, | 0.867 | 0.858 | 0.877 | 0.926 | 0.874 | 0.977 | 0.933 | 0.867 | 0.999 |
| SLC1A2, IL1R2, OLAH, RETN | | | | | | | | | |
| RSAD2, IFIT1, IFI44L, SIGLEC1, | 0.867 | 0.857 | 0.877 | 0.923 | 0.871 | 0.976 | 0.948 | 0.888 | 1 |
| SLC1A2, OLAH, FAM20A, RETN, MMP8 | | | | | | | | | |
| RSAD2, OAS1, IFIT1, SIGLEC1, ISG15, | 0.867 | 0.857 | 0.877 | 0.926 | 0.877 | 0.974 | 0.948 | 0.882 | 1 |
| HERC6, SLC1A2, IL1R2, FAM20A | | | | | | | | | |
| RSAD2, IFI27, OAS1, IFIT1, SIGLEC1, | 0.867 | 0.858 | 0.876 | 0.921 | 0.866 | 0.975 | 0.965 | 0.920 | 1 |
| ISG15, IL1R2, RETN, MMP8 | | | | | | | | | |
| RSAD2, IFIT1, SIGLEC1, ISG15, | 0.867 | 0.858 | 0.876 | 0.928 | 0.882 | 0.975 | 0.960 | 0.894 | 1 |
| SLC1A2, IL1R2, FAM20A, RETN, MMP8 | | | | | | | | | |
| IFI27, IFIT1, IFI44L, SIGLEC1, ISG15, | 0.867 | 0.858 | 0.876 | 0.928 | 0.880 | 0.976 | 0.945 | 0.892 | 0.997 |
| HERC6, SLC1A2, IL1R2, RETN | | | | | | | | | |
| RSAD2, OAS1, IFIT1, SIGLEC1, ISG15, | 0.867 | 0.858 | 0.876 | 0.912 | 0.850 | 0.974 | 0.964 | 0.914 | 1 |
| IL1R2, OLAH, FAM20A, RETN | | | | | | | | | |
| RSAD2, IFI27, OAS1, IFIT1, ISG15, | 0.867 | 0.857 | 0.877 | 0.923 | 0.867 | 0.979 | 0.932 | 0.850 | 1 |
| HERC6, SLC1A2, FAM20A, RETN | | | | | | | | | |
| RSAD2, OAS1, IFIT1, IFI44L, SIGLEC1, | 0.867 | 0.857 | 0.877 | 0.927 | 0.879 | 0.974 | 0.946 | 0.879 | 1 |
| HERC6, SLC1A2, IL1R2, FAM20A | | | | | | | | | |
| IFI27, OAS1, IFIT1, IFI44L, SLC1A2, | 0.867 | 0.857 | 0.877 | 0.924 | 0.872 | 0.977 | 0.942 | 0.881 | 1 |
| IL1R2, OLAH, RETN, MMP8 | | | | | | | | | |
| OAS1, IFI44L, SIGLEC1, HERC6, | 0.867 | 0.857 | 0.877 | 0.926 | 0.878 | 0.975 | 0.959 | 0.895 | 1 |
| SLC1A2, IL1R2, FAM20A, RETN, MMP8 | | | | | | | | | |
| RSAD2, IFIT1, SIGLEC1, HERC6, | 0.867 | 0.857 | 0.877 | 0.924 | 0.874 | 0.974 | 0.947 | 0.876 | 1 |
| SLC1A2, IL1R2, OLAH, FAM20A, RETN | | | | | | | | | |
| OAS1, IFIT1, SIGLEC1, ISG15, HERC6, | 0.867 | 0.857 | 0.877 | 0.921 | 0.868 | 0.975 | 0.943 | 0.873 | 1 |
| SLC1A2, FAM20A, RETN, MMP8 | | | | | | | | | |
| IFI27, OAS1, IFIT1, IFI44L, ISG15, | 0.867 | 0.857 | 0.877 | 0.926 | 0.877 | 0.976 | 0.927 | 0.859 | 0.995 |
| HERC6, SLC1A2, IL1R2, OLAH | | | | | | | | | |
| OAS1, IFIT1, SIGLEC1, ISG15, HERC6, | 0.867 | 0.857 | 0.877 | 0.921 | 0.869 | 0.973 | 0.936 | 0.865 | 1 |
| SLC1A2, OLAH, FAM20A, RETN | | | | | | | | | |
| IFI27, IFIT1, IFI44L, ISG15, HERC6, | 0.867 | 0.857 | 0.876 | 0.928 | 0.880 | 0.977 | 0.930 | 0.866 | 0.995 |
| SLC1A2, IL1R2, OLAH, MMP8 | | | | | | | | | |
| RSAD2, OAS1, IFI44L, SIGLEC1, IL1R2, | 0.867 | 0.856 | 0.877 | 0.919 | 0.861 | 0.976 | 0.962 | 0.906 | 1 |
| OLAH, FAM20A, RETN, MMP8 | | | | | | | | | |
| RSAD2, IFI27, OAS1, IFI44L, ISG15, | 0.867 | 0.858 | 0.876 | 0.929 | 0.880 | 0.978 | 0.930 | 0.862 | 0.998 |
| SLC1A2, IL1R2, OLAH, MMP8 | | | | | | | | | |
| IFI27, OAS1, IFIT1, IFI44L, SIGLEC1, | 0.867 | 0.858 | 0.876 | 0.924 | 0.875 | 0.973 | 0.950 | 0.896 | 1 |
| ISG15, SLC1A2, IL1R2, RETN | | | | | | | | | |
| IFIT1, IFI44L, SIGLEC1, ISG15, | 0.867 | 0.857 | 0.876 | 0.921 | 0.868 | 0.974 | 0.943 | 0.882 | 1 |
| SLC1A2, IL1R2, OLAH, FAM20A, RETN | | | | | | | | | |
| RSAD2, IFIT1, IFI44L, SIGLEC1, | 0.867 | 0.857 | 0.876 | 0.922 | 0.869 | 0.975 | 0.940 | 0.867 | 1 |
| HERC6, SLC1A2, FAM20A, RETN, MMP8 | | | | | | | | | |
| OAS1, IFIT1, IFI44L, SIGLEC1, ISG15, | 0.867 | 0.856 | 0.878 | 0.914 | 0.849 | 0.978 | 0.961 | 0.913 | 1 |
| OLAH, FAM20A, RETN, MMP8 | | | | | | | | | |
| RSAD2, IFI27, IFIT1, IFI44L, ISG15, | 0.867 | 0.857 | 0.877 | 0.922 | 0.867 | 0.977 | 0.928 | 0.846 | 1 |
| HERC6, SLC1A2, FAM20A, RETN | | | | | | | | | |
| RSAD2, IFI27, OAS1, IFI44L, ISG15, | 0.867 | 0.858 | 0.876 | 0.927 | 0.876 | 0.979 | 0.932 | 0.861 | 1 |
| SLC1A2, IL1R2, OLAH, RETN | | | | | | | | | |
| IFI27, IFIT1, SIGLEC1, ISG15, HERC6, | 0.867 | 0.858 | 0.876 | 0.928 | 0.881 | 0.974 | 0.963 | 0.923 | 1 |
| SLC1A2, IL1R2, RETN, MMP8 | | | | | | | | | |
| RSAD2, OAS1, SIGLEC1, HERC6, | 0.867 | 0.857 | 0.877 | 0.921 | 0.867 | 0.974 | 0.948 | 0.882 | 1 |
| SLC1A2, OLAH, FAM20A, RETN, MMP8 | | | | | | | | | |
| RSAD2, IFIT1, IFI44L, SIGLEC1, | 0.867 | 0.857 | 0.876 | 0.929 | 0.882 | 0.975 | 0.961 | 0.900 | 1 |
| SLC1A2, IL1R2, FAM20A, RETN, MMP8 | | | | | | | | | |
| IFI44L, SIGLEC1, HERC6, SLC1A2, | 0.867 | 0.856 | 0.877 | 0.923 | 0.873 | 0.973 | 0.955 | 0.889 | 1 |
| IL1R2, OLAH, FAM20A, RETN, MMP8 | | | | | | | | | |
| RSAD2, IFI27, OAS1, IFIT1, IFI44L, | 0.867 | 0.857 | 0.876 | 0.927 | 0.877 | 0.977 | 0.939 | 0.875 | 1 |
| SLC1A2, IL1R2, OLAH, MMP8 | | | | | | | | | |
| IFIT1, SIGLEC1, ISG15, HERC6, | 0.867 | 0.857 | 0.877 | 0.924 | 0.875 | 0.974 | 0.942 | 0.871 | 1 |
| SLC1A2, IL1R2, OLAH, FAM20A, RETN | | | | | | | | | |
| RSAD2, OAS1, IFI44L, SIGLEC1, ISG15, | 0.867 | 0.857 | 0.877 | 0.918 | 0.860 | 0.976 | 0.961 | 0.902 | 1 |
| IL1R2, FAM20A, RETN, MMP8 | | | | | | | | | |
| IFIT1, IFI44L, SIGLEC1, ISG15, HERC6, | 0.867 | 0.857 | 0.876 | 0.924 | 0.871 | 0.976 | 0.945 | 0.873 | 1 |
| SLC1A2, FAM20A, RETN, MMP8 | | | | | | | | | |
| OAS1, IFIT1, SIGLEC1, HERC6, | 0.867 | 0.857 | 0.877 | 0.924 | 0.875 | 0.973 | 0.942 | 0.872 | 1 |
| SLC1A2, IL1R2, OLAH, FAM20A, RETN | | | | | | | | | |
| RSAD2, IFIT1, IFI44L, SIGLEC1, | 0.867 | 0.856 | 0.877 | 0.925 | 0.875 | 0.975 | 0.928 | 0.855 | 1 |
| HERC6, SLC1A2, OLAH, FAM20A, RETN | | | | | | | | | |
| RSAD2, OAS1, SIGLEC1, ISG15, | 0.867 | 0.856 | 0.877 | 0.919 | 0.867 | 0.972 | 0.945 | 0.876 | 1 |
| HERC6, SLC1A2, OLAH, FAM20A, MMP8 | | | | | | | | | |
| OAS1, IFIT1, IFI44L, SIGLEC1, HERC6, | 0.867 | 0.857 | 0.876 | 0.927 | 0.881 | 0.974 | 0.941 | 0.872 | 1 |
| SLC1A2, IL1R2, FAM20A, RETN | | | | | | | | | |
| RSAD2, IFI27, IFIT1, IFI44L, ISG15, | 0.867 | 0.857 | 0.877 | 0.924 | 0.869 | 0.979 | 0.942 | 0.880 | 1 |
| SLC1A2, OLAH, RETN, MMP8 | | | | | | | | | |
| IFI27, IFIT1, IFI44L, SIGLEC1, ISG15, | 0.867 | 0.858 | 0.875 | 0.925 | 0.877 | 0.973 | 0.958 | 0.912 | 1 |
| SLC1A2, IL1R2, RETN, MMP8 | | | | | | | | | |
| RSAD2, OAS1, IFIT1, SIGLEC1, | 0.867 | 0.857 | 0.876 | 0.927 | 0.879 | 0.974 | 0.961 | 0.900 | 1 |
| SLC1A2, IL1R2, FAM20A, RETN, MMP8 | | | | | | | | | |
| OAS1, IFIT1, SIGLEC1, ISG15, SLC1A2, | 0.867 | 0.856 | 0.877 | 0.922 | 0.869 | 0.975 | 0.951 | 0.891 | 1 |
| OLAH, FAM20A, RETN, MMP8 | | | | | | | | | |
| IFI27, OAS1, IFIT1, IFI44L, ISG15, | 0.867 | 0.856 | 0.877 | 0.925 | 0.871 | 0.979 | 0.941 | 0.877 | 1 |
| SLC1A2, OLAH, RETN, MMP8 | | | | | | | | | |
| IFI27, OAS1, IFI44L, ISG15, HERC6, | 0.867 | 0.856 | 0.877 | 0.925 | 0.869 | 0.981 | 0.935 | 0.854 | 1 |
| SLC1A2, FAM20A, RETN, MMP8 | | | | | | | | | |
| IFI27, OAS1, IFIT1, IFI44L, ISG15, | 0.867 | 0.857 | 0.876 | 0.928 | 0.880 | 0.977 | 0.933 | 0.869 | 0.996 |
| SLC1A2, IL1R2, OLAH, MMP8 | | | | | | | | | |
| RSAD2, IFI27, IFIT1, IFI44L, ISG15, | 0.866 | 0.857 | 0.875 | 0.927 | 0.876 | 0.978 | 0.935 | 0.870 | 1.000 |
| SLC1A2, IL1R2, OLAH, RETN | | | | | | | | | |
| OAS1, IFIT1, SIGLEC1, ISG15, HERC6, | 0.866 | 0.856 | 0.877 | 0.924 | 0.876 | 0.973 | 0.945 | 0.873 | 1 |
| SLC1A2, IL1R2, FAM20A, RETN | | | | | | | | | |
| RSAD2, IFI27, OAS1, IFIT1, HERC6, | 0.866 | 0.857 | 0.876 | 0.923 | 0.872 | 0.974 | 0.940 | 0.876 | 1 |
| SLC1A2, IL1R2, RETN, MMP8 | | | | | | | | | |
| IFI27, OAS1, IFIT1, SIGLEC1, ISG15, | 0.866 | 0.857 | 0.876 | 0.926 | 0.879 | 0.974 | 0.963 | 0.916 | 1 |
| SLC1A2, IL1R2, RETN, MMP8 | | | | | | | | | |
| RSAD2, OAS1, IFIT1, SIGLEC1, HERC6, | 0.866 | 0.857 | 0.876 | 0.922 | 0.868 | 0.975 | 0.943 | 0.873 | 1 |
| SLC1A2, FAM20A, RETN, MMP8 | | | | | | | | | |
| RSAD2, IFI27, IFIT1, IFI44L, ISG15, | 0.866 | 0.857 | 0.876 | 0.928 | 0.879 | 0.977 | 0.929 | 0.864 | 0.995 |
| HERC6, SLC1A2, IL1R2, OLAH | | | | | | | | | |
| RSAD2, IFI27, IFIT1, IFI44L, ISG15, | 0.866 | 0.857 | 0.875 | 0.929 | 0.880 | 0.977 | 0.934 | 0.872 | 0.995 |
| SLC1A2, IL1R2, OLAH, MMP8 | | | | | | | | | |
| RSAD2, IFI27, IFIT1, SIGLEC1, ISG15, | 0.866 | 0.858 | 0.875 | 0.927 | 0.881 | 0.974 | 0.952 | 0.905 | 1.000 |
| HERC6, SLC1A2, IL1R2, RETN | | | | | | | | | |
| RSAD2, IFI27, SIGLEC1, ISG15, HERC6, | 0.866 | 0.857 | 0.875 | 0.927 | 0.879 | 0.974 | 0.962 | 0.921 | 1 |
| SLC1A2, IL1R2, RETN, MMP8 | | | | | | | | | |
| RSAD2, IFI27, OAS1, IFI44L, SIGLEC1, | 0.866 | 0.858 | 0.875 | 0.925 | 0.876 | 0.975 | 0.950 | 0.890 | 1 |
| ISG15, SLC1A2, IL1R2, RETN | | | | | | | | | |
| OAS1, SIGLEC1, ISG15, HERC6, | 0.866 | 0.856 | 0.877 | 0.919 | 0.865 | 0.973 | 0.947 | 0.881 | 1 |
| SLC1A2, OLAH, FAM20A, RETN, MMP8 | | | | | | | | | |
| IFI27, OAS1, IFIT1, ISG15, HERC6, | 0.866 | 0.857 | 0.876 | 0.923 | 0.872 | 0.974 | 0.940 | 0.877 | 1 |
| SLC1A2, IL1R2, RETN, MMP8 | | | | | | | | | |
| RSAD2, OAS1, IFIT1, SIGLEC1, HERC6, | 0.866 | 0.856 | 0.876 | 0.924 | 0.874 | 0.974 | 0.941 | 0.875 | 1 |
| SLC1A2, OLAH, FAM20A, RETN | | | | | | | | | |
| IFI27, OAS1, IFIT1, ISG15, HERC6, | 0.866 | 0.855 | 0.877 | 0.922 | 0.866 | 0.979 | 0.933 | 0.851 | 1 |
| SLC1A2, FAM20A, RETN, MMP8 | | | | | | | | | |
| OAS1, IFIT1, IFI44L, SIGLEC1, ISG15, | 0.866 | 0.856 | 0.876 | 0.924 | 0.874 | 0.974 | 0.935 | 0.865 | 1 |
| HERC6, SLC1A2, FAM20A, RETN | | | | | | | | | |
| RSAD2, IFI27, OAS1, IFI44L, SLC1A2, | 0.866 | 0.856 | 0.876 | 0.928 | 0.876 | 0.980 | 0.940 | 0.875 | 1 |
| IL1R2, OLAH, RETN, MMP8 | | | | | | | | | |
| RSAD2, IFI44L, SIGLEC1, HERC6, | 0.866 | 0.856 | 0.876 | 0.926 | 0.878 | 0.975 | 0.958 | 0.892 | 1 |
| SLC1A2, IL1R2, FAM20A, RETN, MMP8 | | | | | | | | | |
| RSAD2, OAS1, SIGLEC1, ISG15, | 0.866 | 0.856 | 0.876 | 0.925 | 0.877 | 0.973 | 0.959 | 0.895 | 1 |
| SLC1A2, IL1R2, FAM20A, RETN, MMP8 | | | | | | | | | |
| RSAD2, OAS1, SIGLEC1, ISG15, | 0.866 | 0.856 | 0.876 | 0.916 | 0.856 | 0.975 | 0.951 | 0.893 | 1 |
| HERC6, OLAH, FAM20A, RETN, MMP8 | | | | | | | | | |
| IFI27, OAS1, IFI44L, ISG15, SLC1A2, | 0.866 | 0.856 | 0.876 | 0.927 | 0.876 | 0.979 | 0.936 | 0.870 | 1 |
| IL1R2, OLAH, RETN, MMP8 | | | | | | | | | |
| RSAD2, IFI27, OAS1, ISG15, HERC6, | 0.866 | 0.857 | 0.875 | 0.925 | 0.874 | 0.975 | 0.934 | 0.863 | 1 |
| SLC1A2, IL1R2, RETN, MMP8 | | | | | | | | | |
| RSAD2, IFI27, OAS1, IFIT1, IFI44L, | 0.866 | 0.857 | 0.875 | 0.926 | 0.874 | 0.977 | 0.939 | 0.871 | 1 |
| SLC1A2, IL1R2, OLAH, RETN | | | | | | | | | |
| RSAD2, IFI27, OAS1, IFIT1, SIGLEC1, | 0.866 | 0.856 | 0.876 | 0.922 | 0.866 | 0.977 | 0.943 | 0.871 | 1 |
| ISG15, SLC1A2, FAM20A, MMP8 | | | | | | | | | |
| RSAD2, IFI27, OAS1, IFIT1, IFI44L, | 0.866 | 0.857 | 0.875 | 0.925 | 0.876 | 0.974 | 0.950 | 0.889 | 1 |
| SIGLEC1, SLC1A2, IL1R2, RETN | | | | | | | | | |
| RSAD2, IFI44L, SIGLEC1, ISG15, | 0.866 | 0.856 | 0.876 | 0.918 | 0.859 | 0.976 | 0.964 | 0.911 | 1 |
| IL1R2, OLAH, FAM20A, RETN, MMP8 | | | | | | | | | |
| RSAD2, OAS1, IFIT1, IFI44L, SIGLEC1, | 0.866 | 0.856 | 0.876 | 0.910 | 0.847 | 0.974 | 0.961 | 0.908 | 1 |
| IL1R2, OLAH, FAM20A, RETN | | | | | | | | | |
| RSAD2, IFIT1, SIGLEC1, ISG15, | 0.866 | 0.856 | 0.876 | 0.920 | 0.867 | 0.974 | 0.938 | 0.865 | 1 |
| HERC6, SLC1A2, OLAH, FAM20A, RETN | | | | | | | | | |
| RSAD2, IFI27, OAS1, ISG15, SLC1A2, | 0.866 | 0.856 | 0.876 | 0.930 | 0.881 | 0.978 | 0.928 | 0.842 | 1 |
| IL1R2, FAM20A, RETN, MMP8 | | | | | | | | | |
| RSAD2, IFI27, OAS1, IFIT1, IFI44L, | 0.866 | 0.856 | 0.876 | 0.925 | 0.876 | 0.974 | 0.925 | 0.839 | 1 |
| ISG15, SLC1A2, IL1R2, FAM20A | | | | | | | | | |
| RSAD2, IFI27, OAS1, SIGLEC1, ISG15, | 0.866 | 0.855 | 0.877 | 0.919 | 0.865 | 0.974 | 0.940 | 0.864 | 1 |
| HERC6, SLC1A2, FAM20A, MMP8 | | | | | | | | | |
| RSAD2, IFI27, OAS1, IFI44L, ISG15, | 0.866 | 0.856 | 0.876 | 0.927 | 0.874 | 0.981 | 0.939 | 0.874 | 1 |
| SLC1A2, OLAH, RETN, MMP8 | | | | | | | | | |
| RSAD2, IFI27, OAS1, IFI44L, ISG15, | 0.866 | 0.855 | 0.877 | 0.923 | 0.867 | 0.978 | 0.935 | 0.854 | 1 |
| HERC6, SLC1A2, FAM20A, RETN | | | | | | | | | |
| RSAD2, IFI27, OAS1, IFIT1, IFI44L, | 0.866 | 0.856 | 0.875 | 0.927 | 0.877 | 0.978 | 0.932 | 0.865 | 0.998 |
| ISG15, SLC1A2, IL1R2, OLAH | | | | | | | | | |
| RSAD2, IFI27, IFIT1, IFI44L, ISG15, | 0.866 | 0.856 | 0.876 | 0.927 | 0.877 | 0.977 | 0.924 | 0.837 | 1 |
| SLC1A2, IL1R2, FAM20A, RETN | | | | | | | | | |
| RSAD2, IFIT1, SIGLEC1, ISG15, | 0.866 | 0.856 | 0.876 | 0.926 | 0.877 | 0.974 | 0.946 | 0.874 | 1 |
| HERC6, SLC1A2, IL1R2, FAM20A, RETN | | | | | | | | | |
| IFI27, OAS1, IFIT1, IFI44L, HERC6, | 0.866 | 0.856 | 0.875 | 0.923 | 0.872 | 0.974 | 0.937 | 0.871 | 1 |
| SLC1A2, IL1R2, RETN, MMP8 | | | | | | | | | |
| RSAD2, IFI27, OAS1, IFIT1, ISG15, | 0.866 | 0.856 | 0.875 | 0.927 | 0.878 | 0.976 | 0.921 | 0.833 | 1 |
| SLC1A2, IL1R2, FAM20A, RETN | | | | | | | | | |
| IFI27, OAS1, IFIT1, IFI44L, SIGLEC1, | 0.866 | 0.857 | 0.875 | 0.926 | 0.881 | 0.970 | 0.950 | 0.902 | 0.998 |
| HERC6, SLC1A2, IL1R2, MMP8 | | | | | | | | | |
| RSAD2, IFI27, OAS1, IFIT1, ISG15, | 0.866 | 0.857 | 0.875 | 0.924 | 0.873 | 0.975 | 0.935 | 0.865 | 1 |
| HERC6, SLC1A2, IL1R2, RETN | | | | | | | | | |
| RSAD2, IFI27, IFI44L, SIGLEC1, ISG15, | 0.866 | 0.857 | 0.874 | 0.928 | 0.879 | 0.976 | 0.943 | 0.890 | 0.997 |
| HERC6, SLC1A2, IL1R2, RETN | | | | | | | | | |
| RSAD2, IFI27, OAS1, IFIT1, IFI44L, | 0.866 | 0.857 | 0.874 | 0.923 | 0.872 | 0.974 | 0.930 | 0.857 | 1 |
| HERC6, SLC1A2, IL1R2, RETN | | | | | | | | | |
| RSAD2, IFI27, OAS1, IFIT1, IFI44L, | 0.866 | 0.856 | 0.876 | 0.924 | 0.873 | 0.976 | 0.922 | 0.834 | 1 |
| SLC1A2, IL1R2, FAM20A, RETN | | | | | | | | | |
| RSAD2, IFI27, OAS1, IFI44L, SIGLEC1, | 0.866 | 0.857 | 0.875 | 0.927 | 0.879 | 0.975 | 0.957 | 0.902 | 1 |
| SLC1A2, IL1R2, RETN, MMP8 | | | | | | | | | |
| OAS1, IFIT1, IFI44L, SIGLEC1, | 0.866 | 0.856 | 0.875 | 0.922 | 0.870 | 0.975 | 0.946 | 0.885 | 1 |
| SLC1A2, IL1R2, OLAH, FAM20A, RETN | | | | | | | | | |
| RSAD2, OAS1, IFIT1, IFI44L, SIGLEC1, | 0.866 | 0.856 | 0.875 | 0.921 | 0.868 | 0.974 | 0.950 | 0.892 | 1 |
| SLC1A2, IL1R2, OLAH, FAM20A | | | | | | | | | |
| RSAD2, OAS1, IFI44L, SIGLEC1, ISG15, | 0.866 | 0.856 | 0.875 | 0.915 | 0.856 | 0.973 | 0.959 | 0.905 | 1 |
| HERC6, IL1R2, OLAH, FAM20A | | | | | | | | | |
| RSAD2, OAS1, IFIT1, IFI44L, SIGLEC1, | 0.866 | 0.855 | 0.876 | 0.915 | 0.853 | 0.978 | 0.959 | 0.910 | 1 |
| OLAH, FAM20A, RETN, MMP8 | | | | | | | | | |
| RSAD2, IFI27, OAS1, IFI44L, ISG15, | 0.865 | 0.857 | 0.874 | 0.923 | 0.872 | 0.974 | 0.932 | 0.858 | 1 |
| HERC6, SLC1A2, IL1R2, RETN | | | | | | | | | |
| RSAD2, IFI27, OAS1, IFIT1, SIGLEC1, | 0.865 | 0.857 | 0.874 | 0.925 | 0.877 | 0.974 | 0.961 | 0.908 | 1 |
| SLC1A2, IL1R2, RETN, MMP8 | | | | | | | | | |
| IFI27, OAS1, IFI44L, SIGLEC1, ISG15, | 0.865 | 0.857 | 0.874 | 0.927 | 0.881 | 0.974 | 0.959 | 0.910 | 1 |
| SLC1A2, IL1R2, RETN, MMP8 | | | | | | | | | |
| RSAD2, IFI27, OAS1, IFIT1, IFI44L, | 0.865 | 0.857 | 0.874 | 0.926 | 0.880 | 0.972 | 0.954 | 0.906 | 1 |
| SIGLEC1, HERC6, SLC1A2, IL1R2 | | | | | | | | | |
| OAS1, IFI44L, SIGLEC1, ISG15, | 0.865 | 0.856 | 0.875 | 0.925 | 0.877 | 0.974 | 0.958 | 0.894 | 1 |
| SLC1A2, IL1R2, FAM20A, RETN, MMP8 | | | | | | | | | |
| RSAD2, IFI27, OAS1, IFI44L, SIGLEC1, | 0.865 | 0.857 | 0.874 | 0.926 | 0.880 | 0.972 | 0.957 | 0.911 | 1 |
| ISG15, HERC6, SLC1A2, IL1R2 | | | | | | | | | |
| RSAD2, OAS1, IFIT1, SIGLEC1, ISG15, | 0.865 | 0.855 | 0.875 | 0.915 | 0.852 | 0.978 | 0.961 | 0.912 | 1 |
| OLAH, FAM20A, RETN, MMP8 | | | | | | | | | |
| RSAD2, IFI27, OAS1, IFIT1, IFI44L, | 0.865 | 0.857 | 0.874 | 0.919 | 0.869 | 0.970 | 0.951 | 0.905 | 0.997 |
| SIGLEC1, ISG15, IL1R2, MMP8 | | | | | | | | | |
| RSAD2, IFIT1, IFI44L, SIGLEC1, ISG15, | 0.865 | 0.856 | 0.875 | 0.911 | 0.849 | 0.974 | 0.963 | 0.914 | 1 |
| IL1R2, OLAH, FAM20A, RETN | | | | | | | | | |
| OAS1, IFIT1, IFI44L, SIGLEC1, ISG15, | 0.865 | 0.856 | 0.875 | 0.920 | 0.864 | 0.975 | 0.934 | 0.867 | 1 |
| SLC1A2, OLAH, FAM20A, RETN | | | | | | | | | |
| RSAD2, IFIT1, SIGLEC1, ISG15, | 0.865 | 0.856 | 0.875 | 0.921 | 0.868 | 0.975 | 0.943 | 0.871 | 1 |
| HERC6, SLC1A2, FAM20A, RETN, MMP8 | | | | | | | | | |
| RSAD2, OAS1, IFI44L, SIGLEC1, | 0.865 | 0.855 | 0.876 | 0.921 | 0.868 | 0.973 | 0.942 | 0.875 | 1 |
| HERC6, SLC1A2, OLAH, FAM20A, MMP8 | | | | | | | | | |
| RSAD2, IFI27, OAS1, IFIT1, IFI44L, | 0.865 | 0.856 | 0.875 | 0.927 | 0.874 | 0.979 | 0.935 | 0.865 | 1 |
| ISG15, SLC1A2, OLAH, RETN | | | | | | | | | |
| IFI27, OAS1, IFI44L, ISG15, HERC6, | 0.865 | 0.856 | 0.875 | 0.924 | 0.875 | 0.974 | 0.938 | 0.872 | 1 |
| SLC1A2, IL1R2, RETN, MMP8 | | | | | | | | | |
| RSAD2, SIGLEC1, ISG15, HERC6, | 0.865 | 0.855 | 0.875 | 0.920 | 0.866 | 0.974 | 0.943 | 0.873 | 1 |
| SLC1A2, OLAH, FAM20A, RETN, MMP8 | | | | | | | | | |
| RSAD2, IFI27, OAS1, ISG15, HERC6, | 0.865 | 0.854 | 0.876 | 0.922 | 0.865 | 0.978 | 0.933 | 0.851 | 1 |
| SLC1A2, FAM20A, RETN, MMP8 | | | | | | | | | |
| RSAD2, IFIT1, IFI44L, SIGLEC1, | 0.865 | 0.856 | 0.875 | 0.926 | 0.877 | 0.974 | 0.941 | 0.869 | 1 |
| HERC6, SLC1A2, IL1R2, FAM20A, RETN | | | | | | | | | |
| IFI27, OAS1, IFIT1, IFI44L, ISG15, | 0.865 | 0.856 | 0.874 | 0.924 | 0.875 | 0.974 | 0.938 | 0.874 | 1 |
| HERC6, SLC1A2, IL1R2, RETN | | | | | | | | | |
| OAS1, IFIT1, IFI44L, SIGLEC1, ISG15, | 0.865 | 0.855 | 0.875 | 0.919 | 0.866 | 0.973 | 0.942 | 0.879 | 1 |
| SLC1A2, IL1R2, OLAH, FAM20A | | | | | | | | | |
| RSAD2, OAS1, IFIT1, IFI44L, SIGLEC1, | 0.865 | 0.856 | 0.875 | 0.920 | 0.865 | 0.975 | 0.939 | 0.876 | 1 |
| ISG15, SLC1A2, OLAH, FAM20A | | | | | | | | | |
| IFI27, OAS1, IFIT1, IFI44L, SIGLEC1, | 0.865 | 0.856 | 0.874 | 0.926 | 0.881 | 0.970 | 0.949 | 0.901 | 0.997 |
| ISG15, HERC6, SLC1A2, IL1R2 | | | | | | | | | |
| RSAD2, IFI27, OAS1, IFI44L, HERC6, | 0.865 | 0.856 | 0.875 | 0.923 | 0.872 | 0.974 | 0.933 | 0.860 | 1 |
| SLC1A2, IL1R2, RETN, MMP8 | | | | | | | | | |
| RSAD2, IFI27, OAS1, IFIT1, SIGLEC1, | 0.865 | 0.857 | 0.874 | 0.924 | 0.876 | 0.973 | 0.957 | 0.899 | 1 |
| ISG15, SLC1A2, IL1R2, RETN | | | | | | | | | |
| RSAD2, IFI27, OAS1, IFI44L, SIGLEC1, | 0.865 | 0.855 | 0.875 | 0.920 | 0.867 | 0.974 | 0.939 | 0.863 | 1 |
| HERC6, SLC1A2, FAM20A, MMP8 | | | | | | | | | |
| RSAD2, IFI27, OAS1, IFI44L, SIGLEC1, | 0.865 | 0.855 | 0.875 | 0.920 | 0.866 | 0.974 | 0.940 | 0.864 | 1 |
| ISG15, HERC6, SLC1A2, FAM20A | | | | | | | | | |
| RSAD2, OAS1, IFIT1, SIGLEC1, | 0.865 | 0.855 | 0.875 | 0.923 | 0.870 | 0.976 | 0.953 | 0.896 | 1 |
| SLC1A2, OLAH, FAM20A, RETN, MMP8 | | | | | | | | | |
| RSAD2, IFI27, OAS1, IFIT1, SIGLEC1, | 0.865 | 0.856 | 0.874 | 0.926 | 0.881 | 0.972 | 0.951 | 0.901 | 1 |
| HERC6, SLC1A2, IL1R2, MMP8 | | | | | | | | | |
| RSAD2, OAS1, IFI44L, SIGLEC1, ISG15, | 0.865 | 0.855 | 0.875 | 0.918 | 0.863 | 0.973 | 0.945 | 0.881 | 1 |
| SLC1A2, OLAH, FAM20A, MMP8 | | | | | | | | | |
| RSAD2, IFI27, IFIT1, IFI44L, SIGLEC1, | 0.865 | 0.856 | 0.874 | 0.926 | 0.878 | 0.975 | 0.958 | 0.912 | 1 |
| SLC1A2, IL1R2, RETN, MMP8 | | | | | | | | | |
| RSAD2, IFIT1, IFI44L, SIGLEC1, ISG15, | 0.865 | 0.856 | 0.874 | 0.920 | 0.867 | 0.974 | 0.948 | 0.888 | 1 |
| SLC1A2, IL1R2, OLAH, FAM20A | | | | | | | | | |
| IFI27, OAS1, IFIT1, IFI44L, ISG15, | 0.865 | 0.855 | 0.875 | 0.927 | 0.877 | 0.978 | 0.934 | 0.866 | 1 |
| SLC1A2, IL1R2, OLAH, RETN | | | | | | | | | |
| IFI27, OAS1, IFIT1, IFI44L, ISG15, | 0.865 | 0.855 | 0.875 | 0.915 | 0.858 | 0.973 | 0.936 | 0.866 | 1 |
| HERC6, SLC1A2, RETN, MMP8 | | | | | | | | | |
| OAS1, IFIT1, SIGLEC1, ISG15, SLC1A2, | 0.865 | 0.855 | 0.875 | 0.920 | 0.867 | 0.972 | 0.953 | 0.892 | 1 |
| IL1R2, OLAH, FAM20A, RETN | | | | | | | | | |
| RSAD2, OAS1, IFIT1, SIGLEC1, HERC6, | 0.865 | 0.855 | 0.875 | 0.926 | 0.878 | 0.974 | 0.948 | 0.882 | 1 |
| SLC1A2, IL1R2, FAM20A, RETN | | | | | | | | | |
| RSAD2, OAS1, IFIT1, SIGLEC1, ISG15, | 0.865 | 0.855 | 0.875 | 0.919 | 0.864 | 0.973 | 0.957 | 0.896 | 1 |
| SLC1A2, IL1R2, OLAH, FAM20A | | | | | | | | | |
| RSAD2, OAS1, IFI44L, SIGLEC1, ISG15, | 0.865 | 0.855 | 0.875 | 0.924 | 0.876 | 0.972 | 0.959 | 0.893 | 1 |
| SLC1A2, IL1R2, FAM20A, MMP8 | | | | | | | | | |
| RSAD2, IFI27, OAS1, IFI44L, SIGLEC1, | 0.865 | 0.856 | 0.874 | 0.927 | 0.881 | 0.973 | 0.953 | 0.904 | 1 |
| HERC6, SLC1A2, IL1R2, MMP8 | | | | | | | | | |
| OAS1, IFI44L, SIGLEC1, ISG15, HERC6, | 0.865 | 0.854 | 0.876 | 0.919 | 0.866 | 0.972 | 0.942 | 0.874 | 1 |
| SLC1A2, OLAH, FAM20A, MMP8 | | | | | | | | | |
| IFI27, IFIT1, IFI44L, ISG15, HERC6, | 0.865 | 0.854 | 0.875 | 0.922 | 0.864 | 0.979 | 0.936 | 0.854 | 1 |
| SLC1A2, FAM20A, RETN, MMP8 | | | | | | | | | |
| RSAD2, OAS1, IFIT1, SIGLEC1, | 0.865 | 0.855 | 0.874 | 0.922 | 0.869 | 0.974 | 0.957 | 0.898 | 1 |
| SLC1A2, IL1R2, OLAH, FAM20A, RETN | | | | | | | | | |
| RSAD2, IFI27, OAS1, IFIT1, IFI44L, | 0.865 | 0.855 | 0.874 | 0.917 | 0.859 | 0.976 | 0.930 | 0.855 | 1 |
| ISG15, HERC6, SLC1A2, RETN | | | | | | | | | |
| RSAD2, OAS1, IFIT1, IFI44L, SIGLEC1, | 0.865 | 0.855 | 0.875 | 0.911 | 0.850 | 0.972 | 0.935 | 0.866 | 1 |
| ISG15, HERC6, FAM20A, MMP8 | | | | | | | | | |
| RSAD2, IFI27, OAS1, IFI44L, ISG15, | 0.865 | 0.854 | 0.875 | 0.924 | 0.873 | 0.975 | 0.923 | 0.835 | 1 |
| SLC1A2, IL1R2, FAM20A, RETN | | | | | | | | | |
| RSAD2, IFIT1, SIGLEC1, ISG15, | 0.865 | 0.855 | 0.874 | 0.921 | 0.868 | 0.974 | 0.952 | 0.893 | 1 |
| SLC1A2, OLAH, FAM20A, RETN, MMP8 | | | | | | | | | |
| RSAD2, IFI44L, SIGLEC1, SLC1A2, | 0.865 | 0.854 | 0.875 | 0.924 | 0.875 | 0.973 | 0.960 | 0.900 | 1 |
| IL1R2, OLAH, FAM20A, RETN, MMP8 | | | | | | | | | |
| RSAD2, OAS1, IFIT1, IFI44L, SIGLEC1, | 0.865 | 0.855 | 0.874 | 0.911 | 0.848 | 0.973 | 0.963 | 0.911 | 1 |
| ISG15, IL1R2, FAM20A, RETN | | | | | | | | | |
| RSAD2, IFI27, OAS1, IFIT1, SIGLEC1, | 0.865 | 0.856 | 0.873 | 0.926 | 0.881 | 0.971 | 0.953 | 0.905 | 1 |
| ISG15, HERC6, SLC1A2, IL1R2 | | | | | | | | | |
| IFI27, OAS1, IFIT1, SIGLEC1, ISG15, | 0.865 | 0.855 | 0.874 | 0.927 | 0.882 | 0.971 | 0.949 | 0.899 | 0.999 |
| HERC6, SLC1A2, IL1R2, MMP8 | | | | | | | | | |
| RSAD2, IFIT1, SIGLEC1, ISG15, | 0.865 | 0.855 | 0.874 | 0.922 | 0.871 | 0.974 | 0.958 | 0.899 | 1 |
| SLC1A2, IL1R2, OLAH, FAM20A, RETN | | | | | | | | | |
| RSAD2, IFI27, IFIT1, SIGLEC1, ISG15, | 0.864 | 0.856 | 0.873 | 0.928 | 0.881 | 0.974 | 0.964 | 0.922 | 1 |
| SLC1A2, IL1R2, RETN, MMP8 | | | | | | | | | |
| RSAD2, IFIT1, IFI44L, SIGLEC1, | 0.864 | 0.855 | 0.874 | 0.922 | 0.870 | 0.975 | 0.950 | 0.890 | 1 |
| SLC1A2, IL1R2, OLAH, FAM20A, RETN | | | | | | | | | |
| RSAD2, IFI27, OAS1, IFIT1, IFI44L, | 0.864 | 0.854 | 0.875 | 0.915 | 0.856 | 0.973 | 0.917 | 0.837 | 0.998 |
| ISG15, HERC6, FAM20A, MMP8 | | | | | | | | | |
| RSAD2, IFI27, IFIT1, ISG15, HERC6, | 0.864 | 0.854 | 0.875 | 0.924 | 0.869 | 0.980 | 0.935 | 0.853 | 1 |
| SLC1A2, FAM20A, RETN, MMP8 | | | | | | | | | |
| IFI27, OAS1, IFI44L, SIGLEC1, ISG15, | 0.864 | 0.854 | 0.875 | 0.923 | 0.872 | 0.975 | 0.942 | 0.866 | 1 |
| HERC6, SLC1A2, FAM20A, MMP8 | | | | | | | | | |
| OAS1, IFI44L, SIGLEC1, SLC1A2, | 0.864 | 0.854 | 0.875 | 0.922 | 0.872 | 0.973 | 0.958 | 0.899 | 1 |
| IL1R2, OLAH, FAM20A, RETN, MMP8 | | | | | | | | | |
| RSAD2, OAS1, IFIT1, IFI44L, SIGLEC1, | 0.864 | 0.855 | 0.874 | 0.919 | 0.867 | 0.972 | 0.929 | 0.852 | 1 |
| ISG15, HERC6, SLC1A2, FAM20A | | | | | | | | | |
| RSAD2, OAS1, IFIT1, IFI44L, SIGLEC1, | 0.864 | 0.855 | 0.874 | 0.924 | 0.874 | 0.975 | 0.935 | 0.862 | 1 |
| HERC6, SLC1A2, FAM20A, RETN | | | | | | | | | |
| RSAD2, IFI27, IFI44L, SIGLEC1, ISG15, | 0.864 | 0.854 | 0.875 | 0.921 | 0.867 | 0.975 | 0.938 | 0.860 | 1 |
| HERC6, SLC1A2, FAM20A, MMP8 | | | | | | | | | |
| RSAD2, IFI27, IFIT1, IFI44L, HERC6, | 0.864 | 0.854 | 0.874 | 0.923 | 0.868 | 0.979 | 0.938 | 0.856 | 1 |
| SLC1A2, FAM20A, RETN, MMP8 | | | | | | | | | |
| RSAD2, OAS1, IFIT1, IFI44L, SIGLEC1, | 0.864 | 0.854 | 0.874 | 0.920 | 0.869 | 0.972 | 0.935 | 0.859 | 1 |
| HERC6, SLC1A2, FAM20A, MMP8 | | | | | | | | | |
| RSAD2, IFI27, OAS1, IFIT1, IFI44L, | 0.864 | 0.854 | 0.874 | 0.919 | 0.863 | 0.974 | 0.942 | 0.883 | 1 |
| ISG15, IL1R2, RETN, MMP8 | | | | | | | | | |
| RSAD2, OAS1, SIGLEC1, ISG15, | 0.864 | 0.854 | 0.874 | 0.914 | 0.855 | 0.972 | 0.958 | 0.902 | 1 |
| HERC6, IL1R2, OLAH, FAM20A, RETN | | | | | | | | | |
| RSAD2, IFI44L, SIGLEC1, ISG15, | 0.864 | 0.854 | 0.874 | 0.920 | 0.868 | 0.973 | 0.945 | 0.878 | 1 |
| HERC6, SLC1A2, OLAH, FAM20A, MMP8 | | | | | | | | | |
| RSAD2, IFI27, OAS1, IFIT1, IFI44L, | 0.864 | 0.855 | 0.873 | 0.919 | 0.869 | 0.969 | 0.938 | 0.882 | 0.994 |
| ISG15, HERC6, IL1R2, MMP8 | | | | | | | | | |
| RSAD2, OAS1, SIGLEC1, ISG15, | 0.864 | 0.853 | 0.875 | 0.919 | 0.867 | 0.970 | 0.946 | 0.878 | 1 |
| HERC6, SLC1A2, IL1R2, OLAH, FAM20A | | | | | | | | | |
| RSAD2, IFI27, IFIT1, IFI44L, SIGLEC1, | 0.864 | 0.855 | 0.873 | 0.923 | 0.871 | 0.975 | 0.940 | 0.883 | 0.998 |
| ISG15, HERC6, SLC1A2, RETN | | | | | | | | | |
| RSAD2, IFIT1, IFI44L, SIGLEC1, ISG15, | 0.864 | 0.854 | 0.874 | 0.921 | 0.870 | 0.973 | 0.929 | 0.852 | 1 |
| HERC6, SLC1A2, FAM20A, MMP8 | | | | | | | | | |
| OAS1, IFI44L, SIGLEC1, HERC6, | 0.864 | 0.854 | 0.874 | 0.920 | 0.866 | 0.974 | 0.947 | 0.881 | 1 |
| SLC1A2, OLAH, FAM20A, RETN, MMP8 | | | | | | | | | |
| RSAD2, IFI27, OAS1, SIGLEC1, ISG15, | 0.864 | 0.855 | 0.873 | 0.926 | 0.880 | 0.971 | 0.952 | 0.902 | 1 |
| HERC6, SLC1A2, IL1R2, MMP8 | | | | | | | | | |
| RSAD2, IFI44L, SIGLEC1, ISG15, | 0.864 | 0.854 | 0.874 | 0.925 | 0.877 | 0.973 | 0.958 | 0.892 | 1 |
| SLC1A2, IL1R2, FAM20A, RETN, MMP8 | | | | | | | | | |
| RSAD2, OAS1, IFI44L, SIGLEC1, | 0.864 | 0.854 | 0.874 | 0.923 | 0.873 | 0.973 | 0.958 | 0.894 | 1 |
| SLC1A2, IL1R2, FAM20A, RETN, MMP8 | | | | | | | | | |
| OAS1, IFIT1, IFI44L, SIGLEC1, ISG15, | 0.864 | 0.853 | 0.874 | 0.922 | 0.870 | 0.973 | 0.930 | 0.854 | 1 |
| HERC6, SLC1A2, FAM20A, MMP8 | | | | | | | | | |
| RSAD2, IFI27, OAS1, SIGLEC1, ISG15, | 0.864 | 0.855 | 0.873 | 0.926 | 0.878 | 0.974 | 0.960 | 0.906 | 1 |
| SLC1A2, IL1R2, RETN, MMP8 | | | | | | | | | |
| RSAD2, OAS1, IFIT1, SIGLEC1, ISG15, | 0.864 | 0.854 | 0.874 | 0.922 | 0.870 | 0.973 | 0.942 | 0.875 | 1 |
| HERC6, SLC1A2, FAM20A, RETN | | | | | | | | | |
| RSAD2, IFI27, IFI44L, ISG15, HERC6, | 0.864 | 0.853 | 0.875 | 0.925 | 0.869 | 0.980 | 0.938 | 0.860 | 1 |
| SLC1A2, FAM20A, RETN, MMP8 | | | | | | | | | |
| RSAD2, IFIT1, IFI44L, SIGLEC1, ISG15, | 0.864 | 0.854 | 0.873 | 0.921 | 0.865 | 0.976 | 0.937 | 0.872 | 1 |
| SLC1A2, OLAH, FAM20A, RETN | | | | | | | | | |
| RSAD2, IFI27, OAS1, IFI44L, HERC6, | 0.864 | 0.853 | 0.875 | 0.923 | 0.866 | 0.981 | 0.936 | 0.855 | 1 |
| SLC1A2, FAM20A, RETN, MMP8 | | | | | | | | | |
| RSAD2, IFI27, OAS1, IFIT1, HERC6, | 0.864 | 0.853 | 0.874 | 0.923 | 0.866 | 0.980 | 0.932 | 0.848 | 1 |
| SLC1A2, FAM20A, RETN, MMP8 | | | | | | | | | |
| RSAD2, IFIT1, IFI44L, SIGLEC1, ISG15, | 0.864 | 0.854 | 0.873 | 0.924 | 0.874 | 0.974 | 0.934 | 0.858 | 1 |
| HERC6, SLC1A2, FAM20A, RETN | | | | | | | | | |
| OAS1, IFIT1, IFI44L, SIGLEC1, ISG15, | 0.864 | 0.854 | 0.873 | 0.919 | 0.867 | 0.971 | 0.952 | 0.893 | 1 |
| SLC1A2, IL1R2, FAM20A, RETN | | | | | | | | | |
| RSAD2, IFI27, IFI44L, SIGLEC1, ISG15, | 0.864 | 0.855 | 0.872 | 0.928 | 0.881 | 0.975 | 0.959 | 0.914 | 1 |
| SLC1A2, IL1R2, RETN, MMP8 | | | | | | | | | |
| RSAD2, IFI27, IFIT1, IFI44L, SIGLEC1, | 0.864 | 0.855 | 0.872 | 0.924 | 0.875 | 0.974 | 0.947 | 0.893 | 1 |
| ISG15, SLC1A2, IL1R2, RETN | | | | | | | | | |
| RSAD2, IFI27, OAS1, IFIT1, IFI44L, | 0.864 | 0.855 | 0.872 | 0.926 | 0.880 | 0.972 | 0.955 | 0.909 | 1 |
| SIGLEC1, ISG15, SLC1A2, IL1R2 | | | | | | | | | |
| IFI27, OAS1, IFI44L, SIGLEC1, ISG15, | 0.864 | 0.855 | 0.872 | 0.926 | 0.881 | 0.970 | 0.950 | 0.902 | 0.998 |
| HERC6, SLC1A2, IL1R2, MMP8 | | | | | | | | | |
| RSAD2, OAS1, IFIT1, IFI44L, SIGLEC1, | 0.863 | 0.853 | 0.874 | 0.919 | 0.863 | 0.975 | 0.938 | 0.875 | 1 |
| SLC1A2, OLAH, FAM20A, RETN | | | | | | | | | |
| IFI27, OAS1, IFIT1, IFI44L, SIGLEC1, | 0.863 | 0.855 | 0.872 | 0.925 | 0.880 | 0.971 | 0.947 | 0.896 | 0.998 |
| ISG15, SLC1A2, IL1R2, MMP8 | | | | | | | | | |
| RSAD2, IFI27, OAS1, IFIT1, ISG15, | 0.863 | 0.853 | 0.873 | 0.916 | 0.857 | 0.975 | 0.926 | 0.849 | 1 |
| HERC6, SLC1A2, RETN, MMP8 | | | | | | | | | |
| IFI27, IFIT1, IFI44L, SIGLEC1, ISG15, | 0.863 | 0.854 | 0.872 | 0.925 | 0.880 | 0.970 | 0.947 | 0.897 | 0.997 |
| HERC6, SLC1A2, IL1R2, MMP8 | | | | | | | | | |
| RSAD2, OAS1, IFIT1, IFI44L, SIGLEC1, | 0.863 | 0.853 | 0.873 | 0.905 | 0.835 | 0.975 | 0.947 | 0.891 | 1 |
| ISG15, OLAH, FAM20A, RETN | | | | | | | | | |
| RSAD2, IFIT1, IFI44L, SIGLEC1, ISG15, | 0.863 | 0.853 | 0.873 | 0.914 | 0.850 | 0.977 | 0.957 | 0.907 | 1 |
| OLAH, FAM20A, RETN, MMP8 | | | | | | | | | |
| RSAD2, IFI27, IFIT1, IFI44L, SIGLEC1, | 0.863 | 0.854 | 0.872 | 0.925 | 0.879 | 0.971 | 0.949 | 0.901 | 0.996 |
| HERC6, SLC1A2, IL1R2, MMP8 | | | | | | | | | |
| IFI44L, SIGLEC1, ISG15, HERC6, | 0.863 | 0.853 | 0.873 | 10.918 | 0.861 | 0.974 | 0.950 | 0.878 | 1 |
| SLC1A2, OLAH, FAM20A, RETN, MMP8 | | | | | | | | | |
| RSAD2, IFI27, OAS1, IFI44L, SIGLEC1, | 0.863 | 0.855 | 0.871 | 0.926 | 0.880 | 0.973 | 0.953 | 0.906 | 1 |
| ISG15, SLC1A2, IL1R2, MMP8 | | | | | | | | | |
| RSAD2, IFI27, OAS1, IFI44L, ISG15, | 0.863 | 0.853 | 0.873 | 0.916 | 0.857 | 0.974 | 0.928 | 0.851 | 1 |
| HERC6, SLC1A2, RETN, MMP8 | | | | | | | | | |
| RSAD2, IFI27, OAS1, IFIT1, IFI44L, | 0.863 | 0.854 | 0.872 | 0.925 | 0.879 | 0.972 | 0.951 | 0.900 | 1 |
| SIGLEC1, SLC1A2, IL1R2, MMP8 | | | | | | | | | |
| RSAD2, OAS1, IFIT1, IFI44L, SIGLEC1, | 0.863 | 0.853 | 0.872 | 0.921 | 0.869 | 0.972 | 0.954 | 0.895 | 1 |
| ISG15, SLC1A2, IL1R2, FAM20A | | | | | | | | | |
| OAS1, IFI44L, SIGLEC1, ISG15, HERC6, | 0.863 | 0.852 | 0.873 | 0.919 | 0.868 | 0.971 | 0.946 | 0.878 | 1 |
| SLC1A2, IL1R2, OLAH, FAM20A | | | | | | | | | |
| OAS1, IFI44L, SIGLEC1, ISG15, | 0.863 | 0.852 | 0.873 | 0.912 | 0.856 | 0.969 | 0.946 | 0.880 | 1 |
| SLC1A2, OLAH, FAM20A, RETN, MMP8 | | | | | | | | | |
| RSAD2, OAS1, SIGLEC1, ISG15, | 0.863 | 0.852 | 0.873 | 0.917 | 0.862 | 0.972 | 0.950 | 0.886 | 1 |
| SLC1A2, OLAH, FAM20A, RETN, MMP8 | | | | | | | | | |
| RSAD2, OAS1, SIGLEC1, ISG15, | 0.863 | 0.852 | 0.873 | 0.915 | 0.858 | 0.972 | 0.948 | 0.877 | 1 |
| HERC6, SLC1A2, FAM20A, RETN, MMP8 | | | | | | | | | |
| RSAD2, IFI44L, SIGLEC1, ISG15, | 0.863 | 0.852 | 0.873 | 0.919 | 0.866 | 0.971 | 0.946 | 0.873 | 1 |
| HERC6, SLC1A2, IL1R2, OLAH, FAM20A | | | | | | | | | |
| RSAD2, OAS1, IFIT1, SIGLEC1, ISG15, | 0.862 | 0.853 | 0.872 | 0.919 | 0.865 | 0.974 | 0.946 | 0.882 | 1 |
| SLC1A2, OLAH, FAM20A, RETN | | | | | | | | | |
| RSAD2, OAS1, IFIT1, SIGLEC1, ISG15, | 0.862 | 0.853 | 0.872 | 0.922 | 0.870 | 0.973 | 0.957 | 0.895 | 1 |
| SLC1A2, IL1R2, FAM20A, RETN | | | | | | | | | |
| RSAD2, IFIT1, IFI44L, SIGLEC1, ISG15, | 0.862 | 0.853 | 0.872 | 0.920 | 0.869 | 0.971 | 0.954 | 0.892 | 1 |
| SLC1A2, IL1R2, FAM20A, RETN | | | | | | | | | |
| RSAD2, IFI27, IFI44L, SIGLEC1, ISG15, | 0.862 | 0.854 | 0.871 | 0.926 | 0.880 | 0.971 | 0.950 | 0.903 | 0.997 |
| HERC6, SLC1A2, IL1R2, MMP8 | | | | | | | | | |
| RSAD2, OAS1, IFI44L, SIGLEC1, | 0.862 | 0.852 | 0.873 | 0.913 | 0.853 | 0.972 | 0.960 | 0.906 | 1 |
| HERC6, IL1R2, OLAH, FAM20A, RETN | | | | | | | | | |
| RSAD2, IFI44L, SIGLEC1, HERC6, | 0.862 | 0.852 | 0.873 | 0.919 | 0.865 | 0.974 | 0.943 | 0.875 | 1 |
| SLC1A2, OLAH, FAM20A, RETN, MMP8 | | | | | | | | | |
| RSAD2, OAS1, IFIT1, SIGLEC1, ISG15, | 0.862 | 0.852 | 0.872 | 0.918 | 0.864 | 0.971 | 0.937 | 0.860 | 1 |
| HERC6, SLC1A2, FAM20A, MMP8 | | | | | | | | | |
| RSAD2, IFI27, IFIT1, ISG15, HERC6, | 0.862 | 0.853 | 0.871 | 0.924 | 0.874 | 0.974 | 0.943 | 0.885 | 1 |
| SLC1A2, IL1R2, RETN, MMP8 | | | | | | | | | |
| OAS1, IFI44L, SIGLEC1, ISG15, HERC6, | 0.862 | 0.852 | 0.872 | 0.914 | 0.854 | 0.973 | 0.961 | 0.908 | 1 |
| IL1R2, OLAH, FAM20A, RETN | | | | | | | | | |
| RSAD2, IFI27, IFIT1, IFI44L, SIGLEC1, | 0.862 | 0.854 | 0.871 | 0.925 | 0.879 | 0.971 | 0.947 | 0.897 | 0.996 |
| ISG15, HERC6, SLC1A2, IL1R2 | | | | | | | | | |
| RSAD2, IFI27, IFIT1, IFI44L, SIGLEC1, | 0.862 | 0.853 | 0.871 | 0.923 | 0.871 | 0.976 | 0.941 | 0.885 | 0.998 |
| HERC6, SLC1A2, RETN, MMP8 | | | | | | | | | |
| RSAD2, OAS1, IFI44L, SIGLEC1, ISG15, | 0.862 | 0.851 | 0.872 | 0.920 | 0.870 | 0.970 | 0.952 | 0.884 | 1 |
| HERC6, SLC1A2, IL1R2, FAM20A | | | | | | | | | |
| RSAD2, IFI27, OAS1, IFIT1, IFI44L, | 0.862 | 0.852 | 0.872 | 0.914 | 0.855 | 0.974 | 0.926 | 0.847 | 1 |
| HERC6, SLC1A2, RETN, MMP8 | | | | | | | | | |
| RSAD2, IFI44L, SIGLEC1, ISG15, | 0.862 | 0.852 | 0.872 | 0.914 | 0.854 | 0.973 | 0.958 | 0.901 | 1 |
| HERC6, IL1R2, OLAH, FAM20A, RETN | | | | | | | | | |
| RSAD2, IFI27, IFIT1, IFI44L, HERC6, | 0.862 | 0.853 | 0.871 | 0.923 | 0.872 | 0.974 | 0.941 | 0.880 | 1 |
| SLC1A2, IL1R2, RETN, MMP8 | | | | | | | | | |
| RSAD2, IFI27, IFIT1, SIGLEC1, ISG15, | 0.862 | 0.853 | 0.871 | 0.923 | 0.870 | 0.976 | 0.943 | 0.888 | 0.999 |
| HERC6, SLC1A2, RETN, MMP8 | | | | | | | | | |
| RSAD2, IFI27, IFIT1, SIGLEC1, ISG15, | 0.862 | 0.853 | 0.870 | 0.924 | 0.879 | 0.970 | 0.949 | 0.900 | 0.998 |
| HERC6, SLC1A2, IL1R2, MMP8 | | | | | | | | | |
| OAS1, IFI44L, SIGLEC1, ISG15, HERC6, | 0.862 | 0.851 | 0.872 | 0.914 | 0.853 | 0.975 | 0.958 | 0.904 | 1 |
| OLAH, FAM20A, RETN, MMP8 | | | | | | | | | |
| RSAD2, IFI27, OAS1, IFIT1, SIGLEC1, | 0.862 | 0.853 | 0.870 | 0.925 | 0.879 | 0.971 | 0.954 | 0.905 | 1 |
| ISG15, SLC1A2, IL1R2, MMP8 | | | | | | | | | |
| RSAD2, OAS1, IFIT1, IFI44L, SIGLEC1, | 0.861 | 0.852 | 0.871 | 0.921 | 0.869 | 0.972 | 0.953 | 0.894 | 1 |
| SLC1A2, IL1R2, FAM20A, RETN | | | | | | | | | |
| RSAD2, OAS1, IFI44L, SIGLEC1, ISG15, | 0.861 | 0.852 | 0.871 | 0.913 | 0.852 | 0.974 | 0.950 | 0.887 | 1 |
| HERC6, FAM20A, RETN, MMP8 | | | | | | | | | |
| RSAD2, IFI27, OAS1, IFI44L, SIGLEC1, | 0.861 | 0.850 | 0.872 | 0.916 | 0.857 | 0.975 | 0.948 | 0.877 | 1 |
| ISG15, SLC1A2, FAM20A, MMP8 | | | | | | | | | |
| RSAD2, OAS1, IFI44L, SIGLEC1, | 0.861 | 0.851 | 0.872 | 0.915 | 0.855 | 0.975 | 0.951 | 0.893 | 1 |
| HERC6, OLAH, FAM20A, RETN, MMP8 | | | | | | | | | |
| RSAD2, IFI44L, SIGLEC1, ISG15, | 0.861 | 0.851 | 0.872 | 0.914 | 0.858 | 0.971 | 0.945 | 0.881 | 1 |
| SLC1A2, OLAH, FAM20A, RETN, MMP8 | | | | | | | | | |
| IFI44L, SIGLEC1, ISG15, HERC6, | 0.861 | 0.851 | 0.872 | 0.917 | 0.864 | 0.970 | 0.951 | 0.879 | 1 |
| SLC1A2, IL1R2, OLAH, FAM20A, RETN | | | | | | | | | |
| IFI27, IFIT1, IFI44L, SIGLEC1, ISG15, | 0.861 | 0.852 | 0.870 | 0.920 | 0.866 | 0.974 | 0.942 | 0.886 | 0.999 |
| HERC6, SLC1A2, RETN, MMP8 | | | | | | | | | |
| RSAD2, OAS1, IFI44L, SIGLEC1, | 0.861 | 0.850 | 0.872 | 0.919 | 0.867 | 0.970 | 0.946 | 0.878 | 1 |
| HERC6, SLC1A2, IL1R2, OLAH, FAM20A | | | | | | | | | |
| IFI27, IFIT1, IFI44L, ISG15, HERC6, | 0.861 | 0.852 | 0.870 | 0.923 | 0.871 | 0.974 | 0.940 | 0.878 | 1 |
| SLC1A2, IL1R2, RETN, MMP8 | | | | | | | | | |
| RSAD2, IFI27, IFIT1, IFI44L, SIGLEC1, | 0.861 | 0.853 | 0.869 | 0.925 | 0.879 | 0.971 | 0.950 | 0.902 | 0.998 |
| ISG15, SLC1A2, IL1R2, MMP8 | | | | | | | | | |
| RSAD2, OAS1, SIGLEC1, ISG15, | 0.861 | 0.851 | 0.871 | 0.918 | 0.865 | 0.970 | 0.958 | 0.900 | 1 |
| SLC1A2, IL1R2, OLAH, FAM20A, RETN | | | | | | | | | |
| RSAD2, OAS1, IFI44L, SIGLEC1, ISG15, | 0.861 | 0.851 | 0.872 | 0.910 | 0.850 | 0.970 | 0.953 | 0.898 | 1 |
| HERC6, OLAH, FAM20A, MMP8 | | | | | | | | | |
| RSAD2, OAS1, SIGLEC1, HERC6, | 0.861 | 0.850 | 0.872 | 0.919 | 0.868 | 0.970 | 0.949 | 0.883 | 1 |
| SLC1A2, IL1R2, OLAH, FAM20A, RETN | | | | | | | | | |
| RSAD2, IFI27, IFIT1, IFI44L, ISG15, | 0.861 | 0.852 | 0.870 | 0.923 | 0.872 | 0.973 | 0.934 | 0.869 | 0.999 |
| HERC6, SLC1A2, IL1R2, RETN | | | | | | | | | |
| RSAD2, SIGLEC1, ISG15, HERC6, | 0.861 | 0.851 | 0.872 | 0.919 | 0.868 | 0.970 | 0.950 | 0.879 | 1 |
| SLC1A2, IL1R2, OLAH, FAM20A, RETN | | | | | | | | | |
| OAS1, SIGLEC1, ISG15, HERC6, | 0.861 | 0.850 | 0.872 | 0.920 | 0.870 | 0.971 | 0.949 | 0.882 | 1 |
| SLC1A2, IL1R2, OLAH, FAM20A, RETN | | | | | | | | | |
| RSAD2, OAS1, IFI44L, SIGLEC1, ISG15, | 0.861 | 0.851 | 0.871 | 0.914 | 0.854 | 0.973 | 0.959 | 0.904 | 1 |
| HERC6, IL1R2, FAM20A, RETN | | | | | | | | | |
| RSAD2, IFI27, IFI44L, ISG15, HERC6, | 0.861 | 0.852 | 0.870 | 0.924 | 0.874 | 0.974 | 0.935 | 0.868 | 1 |
| SLC1A2, IL1R2, RETN, MMP8 | | | | | | | | | |
| RSAD2, OAS1, IFI44L, SIGLEC1, ISG15, | 0.861 | 0.850 | 0.871 | 0.905 | 0.841 | 0.969 | 0.962 | 0.907 | 1 |
| IL1R2, OLAH, FAM20A, RETN | | | | | | | | | |
| RSAD2, IFI44L, SIGLEC1, ISG15, | 0.861 | 0.851 | 0.871 | 0.916 | 0.857 | 0.976 | 0.952 | 0.895 | 1 |
| HERC6, OLAH, FAM20A, RETN, MMP8 | | | | | | | | | |
| RSAD2, OAS1, IFI44L, SIGLEC1, ISG15, | 0.861 | 0.850 | 0.871 | 0.918 | 0.863 | 0.972 | 0.942 | 0.873 | 1 |
| HERC6, SLC1A2, OLAH, FAM20A | | | | | | | | | |
| RSAD2, OAS1, IFI44L, SIGLEC1, | 0.861 | 0.850 | 0.871 | 0.915 | 0.858 | 0.971 | 0.947 | 0.884 | 1 |
| SLC1A2, OLAH, FAM20A, RETN, MMP8 | | | | | | | | | |
| RSAD2, IFI27, OAS1, IFIT1, IFI44L, | 0.860 | 0.851 | 0.870 | 0.919 | 0.867 | 0.972 | 0.938 | 0.872 | 1 |
| SLC1A2, IL1R2, RETN, MMP8 | | | | | | | | | |
| RSAD2, IFI27, OAS1, IFIT1, IFI44L, | 0.860 | 0.851 | 0.869 | 0.922 | 0.875 | 0.968 | 0.936 | 0.873 | 0.999 |
| ISG15, HERC6, SLC1A2, IL1R2 | | | | | | | | | |
| RSAD2, OAS1, SIGLEC1, ISG15, | 0.860 | 0.850 | 0.871 | 0.918 | 0.868 | 0.969 | 0.952 | 0.884 | 1 |
| HERC6, SLC1A2, IL1R2, FAM20A, RETN | | | | | | | | | |
| RSAD2, IFI44L, SIGLEC1, ISG15, | 0.860 | 0.850 | 0.870 | 0.917 | 0.864 | 0.969 | 0.954 | 0.891 | 1 |
| SLC1A2, IL1R2, OLAH, FAM20A, RETN | | | | | | | | | |
| RSAD2, IFI27, OAS1, IFIT1, ISG15, | 0.860 | 0.851 | 0.869 | 0.919 | 0.867 | 0.972 | 0.942 | 0.879 | 1 |
| SLC1A2, IL1R2, RETN, MMP8 | | | | | | | | | |
| OAS1, IFI44L, SIGLEC1, ISG15, HERC6, | 0.860 | 0.849 | 0.871 | 0.920 | 0.869 | 0.970 | 0.949 | 0.880 | 1 |
| SLC1A2, IL1R2, FAM20A, RETN | | | | | | | | | |
| RSAD2, IFI27, OAS1, IFIT1, IFI44L, | 0.860 | 0.851 | 0.869 | 0.922 | 0.875 | 0.969 | 0.935 | 0.870 | 1.000 |
| HERC6, SLC1A2, IL1R2, MMP8 | | | | | | | | | |
| RSAD2, IFI27, OAS1, IFIT1, IFI44L, | 0.860 | 0.851 | 0.869 | 0.920 | 0.868 | 0.972 | 0.934 | 0.862 | 1 |
| ISG15, SLC1A2, IL1R2, RETN | | | | | | | | | |
| IFI27, OAS1, IFIT1, IFI44L, ISG15, | 0.860 | 0.850 | 0.870 | 0.921 | 0.870 | 0.972 | 0.940 | 0.878 | 1 |
| SLC1A2, IL1R2, RETN, MMP8 | | | | | | | | | |
| RSAD2, IFI27, OAS1, IFIT1, ISG15, | 0.860 | 0.851 | 0.869 | 0.923 | 0.876 | 0.969 | 0.937 | 0.873 | 1 |
| HERC6, SLC1A2, IL1R2, MMP8 | | | | | | | | | |
| OAS1, IFI44L, SIGLEC1, ISG15, | 0.860 | 0.849 | 0.870 | 0.918 | 0.866 | 0.970 | 0.955 | 0.894 | 1 |
| SLC1A2, IL1R2, OLAH, FAM20A, RETN | | | | | | | | | |
| RSAD2, IFI27, OAS1, IFIT1, IFI44L, | 0.860 | 0.849 | 0.870 | 0.909 | 0.847 | 0.971 | 0.952 | 0.889 | 1 |
| SIGLEC1, ISG15, RETN, MMP8 | | | | | | | | | |
| RSAD2, IFI27, OAS1, IFIT1, IFI44L, | 0.860 | 0.851 | 0.868 | 0.915 | 0.857 | 0.973 | 0.946 | 0.876 | 1 |
| SIGLEC1, ISG15, SLC1A2, RETN | | | | | | | | | |
| RSAD2, OAS1, SIGLEC1, ISG15, | 0.859 | 0.849 | 0.870 | 0.915 | 0.860 | 0.970 | 0.943 | 0.875 | 1 |
| HERC6, SLC1A2, OLAH, FAM20A, RETN | | | | | | | | | |
| RSAD2, OAS1, IFIT1, IFI44L, SIGLEC1, | 0.859 | 0.849 | 0.870 | 0.910 | 0.845 | 0.976 | 0.941 | 0.874 | 1 |
| ISG15, FAM20A, RETN, MMP8 | | | | | | | | | |
| RSAD2, IFI27, IFI44L, SIGLEC1, ISG15, | 0.859 | 0.850 | 0.869 | 0.921 | 0.867 | 0.974 | 0.943 | 0.887 | 1 |
| HERC6, SLC1A2, RETN, MMP8 | | | | | | | | | |
| OAS1, IFI44L, SIGLEC1, HERC6, | 0.859 | 0.848 | 0.870 | 0.920 | 0.870 | 0.971 | 0.948 | 0.880 | 1 |
| SLC1A2, IL1R2, OLAH, FAM20A, RETN | | | | | | | | | |
| OAS1, IFIT1, IFI44L, SIGLEC1, ISG15, | 0.859 | 0.849 | 0.870 | 0.918 | 0.862 | 0.974 | 0.938 | 0.865 | 1 |
| SLC1A2, FAM20A, RETN, MMP8 | | | | | | | | | |
| RSAD2, OAS1, IFI44L, SIGLEC1, ISG15, | 0.859 | 0.849 | 0.869 | 0.917 | 0.863 | 0.971 | 0.955 | 0.896 | 1 |
| SLC1A2, IL1R2, OLAH, FAM20A | | | | | | | | | |
| RSAD2, OAS1, IFIT1, IFI44L, HERC6, | 0.859 | 0.849 | 0.869 | 0.912 | 0.854 | 0.970 | 0.936 | 0.860 | 1 |
| IL1R2, OLAH, FAM20A, MMP8 | | | | | | | | | |
| RSAD2, IFI27, IFIT1, IFI44L, ISG15, | 0.859 | 0.850 | 0.868 | 0.922 | 0.871 | 0.973 | 0.940 | 0.880 | 1 |
| SLC1A2, IL1R2, RETN, MMP8 | | | | | | | | | |
| IFIT1, IFI44L, ISG15, HERC6, SLC1A2, | 0.859 | 0.850 | 0.869 | 0.917 | 0.866 | 0.968 | 0.929 | 0.847 | 1 |
| IL1R2, OLAH, FAM20A, MMP8 | | | | | | | | | |
| RSAD2, IFI27, OAS1, IFIT1, IFI44L, | 0.859 | 0.848 | 0.871 | 0.916 | 0.854 | 0.978 | 0.947 | 0.873 | 1 |
| ISG15, FAM20A, RETN, MMP8 | | | | | | | | | |
| RSAD2, OAS1, IFI44L, SIGLEC1, | 0.859 | 0.849 | 0.869 | 0.920 | 0.869 | 0.970 | 0.952 | 0.884 | 1 |
| HERC6, SLC1A2, IL1R2, FAM20A, RETN | | | | | | | | | |
| RSAD2, OAS1, IFI44L, HERC6, | 0.859 | 0.848 | 0.870 | 0.920 | 0.870 | 0.970 | 0.927 | 0.845 | 1 |
| SLC1A2, IL1R2, OLAH, FAM20A, MMP8 | | | | | | | | | |
| RSAD2, IFI44L, SIGLEC1, ISG15, | 0.859 | 0.848 | 0.869 | 0.918 | 0.866 | 0.969 | 0.948 | 0.875 | 1 |
| HERC6, SLC1A2, IL1R2, FAM20A, RETN | | | | | | | | | |
| OAS1, IFI44L, SIGLEC1, ISG15, HERC6, | 0.859 | 0.848 | 0.869 | 0.916 | 0.862 | 0.971 | 0.941 | 0.872 | 1 |
| SLC1A2, OLAH, FAM20A, RETN | | | | | | | | | |
| RSAD2, OAS1, IFI44L, SIGLEC1, | 0.859 | 0.848 | 0.869 | 0.917 | 0.863 | 0.970 | 0.958 | 0.900 | 1 |
| SLC1A2, IL1R2, OLAH, FAM20A, RETN | | | | | | | | | |
| IFI27, OAS1, IFIT1, IFI44L, SIGLEC1, | 0.859 | 0.849 | 0.868 | 0.916 | 0.859 | 0.972 | 0.950 | 0.885 | 1 |
| ISG15, SLC1A2, RETN, MMP8 | | | | | | | | | |
| RSAD2, IFI44L, SIGLEC1, HERC6, | 0.859 | 0.847 | 0.870 | 0.920 | 0.869 | 0.971 | 0.947 | 0.877 | 1 |
| SLC1A2, IL1R2, OLAH, FAM20A, RETN | | | | | | | | | |
| RSAD2, OAS1, IFIT1, IFI44L, SIGLEC1, | 0.859 | 0.848 | 0.869 | 0.913 | 0.855 | 0.971 | 0.936 | 0.861 | 1 |
| ISG15, SLC1A2, FAM20A, MMP8 | | | | | | | | | |
| RSAD2, OAS1, IFI44L, SIGLEC1, ISG15, | 0.859 | 0.848 | 0.869 | 0.908 | 0.843 | 0.973 | 0.965 | 0.922 | 1 |
| OLAH, FAM20A, RETN, MMP8 | | | | | | | | | |
| IFI27, OAS1, IFIT1, IFI44L, ISG15, | 0.859 | 0.848 | 0.870 | 0.915 | 0.861 | 0.970 | 0.910 | 0.820 | 1.000 |
| HERC6, SLC1A2, FAM20A, MMP8 | | | | | | | | | |
| RSAD2, IFI27, OAS1, IFI44L, ISG15, | 0.859 | 0.849 | 0.868 | 0.923 | 0.877 | 0.970 | 0.928 | 0.858 | 0.999 |
| HERC6, SLC1A2, IL1R2, MMP8 | | | | | | | | | |
| OAS1, IFI44L, SIGLEC1, ISG15, HERC6, | 0.859 | 0.848 | 0.869 | 0.914 | 0.856 | 0.972 | 0.950 | 0.883 | 1 |
| SLC1A2, FAM20A, RETN, MMP8 | | | | | | | | | |
| RSAD2, IFI44L, SIGLEC1, ISG15, | 0.859 | 0.848 | 0.869 | 0.915 | 0.858 | 0.973 | 0.948 | 0.875 | 1 |
| HERC6, SLC1A2, FAM20A, RETN, MMP8 | | | | | | | | | |
| RSAD2, OAS1, IFIT1, IFI44L, HERC6, | 0.858 | 0.848 | 0.869 | 0.918 | 0.866 | 0.970 | 0.918 | 0.836 | 1 |
| SLC1A2, OLAH, FAM20A, MMP8 | | | | | | | | | |
| OAS1, IFIT1, IFI44L, HERC6, SLC1A2, | 0.858 | 0.848 | 0.869 | 0.920 | 0.867 | 0.974 | 0.917 | 0.830 | 1 |
| OLAH, FAM20A, RETN, MMP8 | | | | | | | | | |
| RSAD2, IFIT1, IFI44L, SIGLEC1, ISG15, | 0.858 | 0.848 | 0.868 | 0.917 | 0.860 | 0.973 | 0.937 | 0.865 | 1 |
| SLC1A2, FAM20A, RETN, MMP8 | | | | | | | | | |
| IFI27, OAS1, IFIT1, IFI44L, ISG15, | 0.858 | 0.849 | 0.868 | 0.923 | 0.878 | 0.969 | 0.936 | 0.875 | 0.997 |
| HERC6, SLC1A2, IL1R2, MMP8 | | | | | | | | | |
| RSAD2, OAS1, ISG15, HERC6, IL1R2, | 0.858 | 0.848 | 0.869 | 0.911 | 0.852 | 0.970 | 0.942 | 0.867 | 1 |
| OLAH, FAM20A, RETN, MMP8 | | | | | | | | | |
| RSAD2, OAS1, IFI44L, ISG15, HERC6, | 0.858 | 0.847 | 0.869 | 0.912 | 0.855 | 0.970 | 0.933 | 0.855 | 1 |
| IL1R2, OLAH, FAM20A, MMP8 | | | | | | | | | |
| RSAD2, OAS1, IFI44L, SIGLEC1, ISG15, | 0.858 | 0.848 | 0.869 | 0.915 | 0.861 | 0.968 | 0.954 | 0.888 | 1 |
| SLC1A2, IL1R2, FAM20A, RETN | | | | | | | | | |
| RSAD2, IFIT1, IFI44L, HERC6, SLC1A2, | 0.858 | 0.848 | 0.868 | 0.919 | 0.869 | 0.970 | 0.926 | 0.844 | 1 |
| IL1R2, OLAH, FAM20A, MMP8 | | | | | | | | | |
| OAS1, IFIT1, IFI44L, ISG15, HERC6, | 0.858 | 0.847 | 0.869 | 0.918 | 0.865 | 0.970 | 0.916 | 0.830 | 1 |
| SLC1A2, OLAH, FAM20A, MMP8 | | | | | | | | | |
| IFIT1, IFI44L, HERC6, SLC1A2, IL1R2, | 0.858 | 0.848 | 0.868 | 0.920 | 0.869 | 0.972 | 0.929 | 0.845 | 1 |
| OLAH, FAM20A, RETN, MMP8 | | | | | | | | | |
| IFI44L, ISG15, HERC6, SLC1A2, IL1R2, | 0.858 | 0.848 | 0.868 | 0.919 | 0.867 | 0.971 | 0.930 | 0.847 | 1 |
| OLAH, FAM20A, RETN, MMP8 | | | | | | | | | |
| RSAD2, IFI27, IFIT1, IFI44L, ISG15, | 0.858 | 0.847 | 0.869 | 0.916 | 0.861 | 0.972 | 0.912 | 0.823 | 1 |
| HERC6, SLC1A2, FAM20A, MMP8 | | | | | | | | | |
| IFIT1, IFI44L, ISG15, HERC6, SLC1A2, | 0.858 | 0.847 | 0.868 | 0.919 | 0.864 | 0.973 | 0.924 | 0.842 | 1 |
| OLAH, FAM20A, RETN, MMP8 | | | | | | | | | |
| RSAD2, IFI27, OAS1, IFI44L, ISG15, | 0.858 | 0.849 | 0.866 | 0.924 | 0.874 | 0.974 | 0.934 | 0.863 | 1 |
| SLC1A2, IL1R2, RETN, MMP8 | | | | | | | | | |
| OAS1, IFIT1, IFI44L, ISG15, HERC6, | 0.858 | 0.847 | 0.868 | 0.917 | 0.864 | 0.970 | 0.917 | 0.831 | 1 |
| SLC1A2, OLAH, FAM20A, RETN | | | | | | | | | |
| RSAD2, OAS1, IFI44L, SIGLEC1, | 0.858 | 0.847 | 0.868 | 0.913 | 0.855 | 0.971 | 0.949 | 0.878 | 1 |
| HERC6, SLC1A2, FAM20A, RETN, MMP8 | | | | | | | | | |
| RSAD2, OAS1, IFI44L, SIGLEC1, | 0.857 | 0.847 | 0.868 | 0.917 | 0.863 | 0.972 | 0.941 | 0.872 | 1 |
| HERC6, SLC1A2, OLAH, FAM20A, RETN | | | | | | | | | |
| RSAD2, IFI27, OAS1, IFIT1, IFI44L, | 0.857 | 0.848 | 0.867 | 0.916 | 0.859 | 0.974 | 0.948 | 0.878 | 1 |
| SIGLEC1, SLC1A2, RETN, MMP8 | | | | | | | | | |
| RSAD2, IFI27, OAS1, IFIT1, SIGLEC1, | 0.857 | 0.848 | 0.867 | 0.915 | 0.857 | 0.973 | 0.949 | 0.880 | 1 |
| ISG15, SLC1A2, RETN, MMP8 | | | | | | | | | |
| OAS1, IFI44L, ISG15, HERC6, SLC1A2, | 0.857 | 0.846 | 0.868 | 0.917 | 0.865 | 0.969 | 0.929 | 0.847 | 1 |
| IL1R2, OLAH, FAM20A, MMP8 | | | | | | | | | |
| RSAD2, IFI27, OAS1, IFIT1, IFI44L, | 0.857 | 0.847 | 0.868 | 0.917 | 0.864 | 0.971 | 0.908 | 0.817 | 0.998 |
| HERC6, SLC1A2, FAM20A, MMP8 | | | | | | | | | |
| RSAD2, OAS1, IFI44L, SIGLEC1, ISG15, | 0.857 | 0.847 | 0.867 | 0.908 | 0.846 | 0.970 | 0.951 | 0.896 | 1 |
| HERC6, OLAH, FAM20A, RETN | | | | | | | | | |
| OAS1, IFIT1, IFI44L, HERC6, SLC1A2, | 0.857 | 0.847 | 0.868 | 0.918 | 0.866 | 0.969 | 0.926 | 0.840 | 1 |
| IL1R2, OLAH, FAM20A, MMP8 | | | | | | | | | |
| RSAD2, IFIT1, IFI44L, HERC6, SLC1A2, | 0.857 | 0.847 | 0.867 | 0.919 | 0.866 | 0.972 | 0.922 | 0.838 | 1 |
| OLAH, FAM20A, RETN, MMP8 | | | | | | | | | |
| RSAD2, IFI27, OAS1, IFIT1, IFI44L, | 0.857 | 0.848 | 0.866 | 0.916 | 0.863 | 0.969 | 0.921 | 0.848 | 0.994 |
| SIGLEC1, HERC6, SLC1A2, MMP8 | | | | | | | | | |
| OAS1, IFIT1, SIGLEC1, ISG15, HERC6, | 0.857 | 0.847 | 0.867 | 0.915 | 0.861 | 0.970 | 0.955 | 0.911 | 1 |
| SLC1A2, OLAH, RETN, MMP8 | | | | | | | | | |
| RSAD2, IFI27, OAS1, IFIT1, IFI44L, | 0.857 | 0.848 | 0.866 | 0.922 | 0.875 | 0.969 | 0.938 | 0.874 | 1 |
| ISG15, SLC1A2, IL1R2, MMP8 | | | | | | | | | |
| RSAD2, OAS1, IFIT1, IFI44L, SIGLEC1, | 0.857 | 0.847 | 0.867 | 0.904 | 0.842 | 0.966 | 0.964 | 0.927 | 1 |
| HERC6, IL1R2, RETN, MMP8 | | | | | | | | | |
| OAS1, IFIT1, SIGLEC1, ISG15, HERC6, | 0.857 | 0.847 | 0.867 | 0.907 | 0.847 | 0.967 | 0.963 | 0.925 | 1 |
| IL1R2, OLAH, RETN, MMP8 | | | | | | | | | |
| RSAD2, OAS1, IFI44L, SIGLEC1, ISG15, | 0.857 | 0.846 | 0.867 | 0.915 | 0.858 | 0.973 | 0.947 | 0.885 | 1 |
| SLC1A2, OLAH, FAM20A, RETN | | | | | | | | | |
| RSAD2, OAS1, IFIT1, SIGLEC1, ISG15, | 0.857 | 0.847 | 0.867 | 0.906 | 0.845 | 0.966 | 0.967 | 0.932 | 1 |
| HERC6, IL1R2, RETN, MMP8 | | | | | | | | | |
| RSAD2, IFI27, OAS1, IFIT1, SIGLEC1, | 0.857 | 0.848 | 0.866 | 0.916 | 0.864 | 0.968 | 0.912 | 0.837 | 0.987 |
| ISG15, HERC6, SLC1A2, MMP8 | | | | | | | | | |
| RSAD2, IFIT1, IFI44L, ISG15, HERC6, | 0.857 | 0.846 | 0.867 | 0.918 | 0.865 | 0.970 | 0.917 | 0.834 | 1 |
| SLC1A2, OLAH, FAM20A, MMP8 | | | | | | | | | |
| RSAD2, OAS1, IFIT1, ISG15, HERC6, | 0.857 | 0.847 | 0.867 | 0.913 | 0.854 | 0.971 | 0.939 | 0.862 | 1 |
| IL1R2, FAM20A, RETN, MMP8 | | | | | | | | | |
| RSAD2, OAS1, IFIT1, IFI44L, SIGLEC1, | 0.857 | 0.846 | 0.867 | 0.919 | 0.863 | 0.975 | 0.938 | 0.867 | 1 |
| SLC1A2, FAM20A, RETN, MMP8 | | | | | | | | | |
| RSAD2, OAS1, IFI44L, HERC6, IL1R2, | 0.857 | 0.846 | 0.867 | 0.914 | 0.855 | 0.972 | 0.938 | 0.864 | 1 |
| OLAH, FAM20A, RETN, MMP8 | | | | | | | | | |
| OAS1, IFIT1, ISG15, HERC6, IL1R2, | 0.857 | 0.847 | 0.867 | 0.909 | 0.848 | 0.969 | 0.943 | 0.871 | 1 |
| OLAH, FAM20A, RETN, MMP8 | | | | | | | | | |
| RSAD2, IFI27, OAS1, IFI44L, SIGLEC1, | 0.857 | 0.847 | 0.866 | 0.916 | 0.858 | 0.974 | 0.948 | 0.876 | 1 |
| ISG15, SLC1A2, RETN, MMP8 | | | | | | | | | |
| RSAD2, IFI44L, SIGLEC1, ISG15, | 0.857 | 0.846 | 0.867 | 0.916 | 0.861 | 0.971 | 0.938 | 0.867 | 1 |
| HERC6, SLC1A2, OLAH, FAM20A, RETN | | | | | | | | | |
| OAS1, IFIT1, IFI44L, SIGLEC1, ISG15, | 0.857 | 0.847 | 0.867 | 0.907 | 0.847 | 0.967 | 0.962 | 0.923 | 1 |
| HERC6, IL1R2, RETN, MMP8 | | | | | | | | | |
| OAS1, IFIT1, IFI44L, ISG15, HERC6, | 0.857 | 0.846 | 0.867 | 0.918 | 0.866 | 0.969 | 0.921 | 0.834 | 1 |
| SLC1A2, IL1R2, OLAH, FAM20A | | | | | | | | | |
| RSAD2, IFI44L, HERC6, SLC1A2, | 0.856 | 0.846 | 0.867 | 0.919 | 0.867 | 0.971 | 0.929 | 0.847 | 1 |
| IL1R2, OLAH, FAM20A, RETN, MMP8 | | | | | | | | | |
| OAS1, IFIT1, IFI44L, SIGLEC1, HERC6, | 0.856 | 0.846 | 0.867 | 0.916 | 0.862 | 0.971 | 0.951 | 0.904 | 0.999 |
| SLC1A2, OLAH, RETN, MMP8 | | | | | | | | | |
| RSAD2, OAS1, IFIT1, ISG15, HERC6, | 0.856 | 0.846 | 0.867 | 0.910 | 0.850 | 0.969 | 0.941 | 0.867 | 1 |
| IL1R2, OLAH, FAM20A, MMP8 | | | | | | | | | |
| OAS1, IFIT1, IFI44L, SIGLEC1, HERC6, | 0.856 | 0.846 | 0.866 | 0.904 | 0.841 | 0.967 | 0.964 | 0.926 | 1 |
| IL1R2, OLAH, RETN, MMP8 | | | | | | | | | |
| OAS1, IFIT1, SIGLEC1, HERC6, | 0.856 | 0.846 | 0.867 | 0.915 | 0.861 | 0.969 | 0.958 | 0.916 | 0.999 |
| SLC1A2, IL1R2, OLAH, RETN, MMP8 | | | | | | | | | |
| RSAD2, OAS1, IFIT1, IFI44L, ISG15, | 0.856 | 0.846 | 0.867 | 0.911 | 0.853 | 0.968 | 0.932 | 0.855 | 1 |
| HERC6, IL1R2, OLAH, FAM20A | | | | | | | | | |
| RSAD2, OAS1, IFIT1, IFI44L, SLC1A2, | 0.856 | 0.846 | 0.866 | 0.917 | 0.865 | 0.969 | 0.933 | 0.852 | 1 |
| IL1R2, OLAH, FAM20A, MMP8 | | | | | | | | | |
| OAS1, IFIT1, IFI44L, ISG15, HERC6, | 0.856 | 0.846 | 0.867 | 0.909 | 0.850 | 0.968 | 0.937 | 0.860 | 1 |
| IL1R2, OLAH, FAM20A, MMP8 | | | | | | | | | |
| OAS1, IFI44L, ISG15, HERC6, SLC1A2, | 0.856 | 0.846 | 0.867 | 0.919 | 0.864 | 0.974 | 0.930 | 0.852 | 1 |
| OLAH, FAM20A, RETN, MMP8 | | | | | | | | | |
| RSAD2, IFI27, IFIT1, IFI44L, ISG15, | 0.856 | 0.847 | 0.865 | 0.923 | 0.878 | 0.969 | 0.935 | 0.875 | 0.995 |
| HERC6, SLC1A2, IL1R2, MMP8 | | | | | | | | | |
| RSAD2, OAS1, IFIT1, HERC6, IL1R2, | 0.856 | 0.846 | 0.866 | 0.910 | 0.850 | 0.970 | 0.943 | 0.870 | 1 |
| OLAH, FAM20A, RETN, MMP8 | | | | | | | | | |
| OAS1, IFIT1, IFI44L, HERC6, IL1R2, | 0.856 | 0.846 | 0.866 | 0.911 | 0.851 | 0.971 | 0.942 | 0.869 | 1 |
| OLAH, FAM20A, RETN, MMP8 | | | | | | | | | |
| IFIT1, IFI44L, ISG15, HERC6, IL1R2, | 0.856 | 0.846 | 0.866 | 0.911 | 0.851 | 0.971 | 0.941 | 0.867 | 1 |
| OLAH, FAM20A, RETN, MMP8 | | | | | | | | | |
| RSAD2, OAS1, IFIT1, HERC6, SLC1A2, | 0.856 | 0.846 | 0.866 | 0.917 | 0.866 | 0.968 | 0.933 | 0.852 | 1 |
| IL1R2, OLAH, FAM20A, MMP8 | | | | | | | | | |
| RSAD2, IFI27, OAS1, IFIT1, IFI44L, | 0.856 | 0.847 | 0.865 | 0.907 | 0.850 | 0.964 | 0.908 | 0.832 | 0.983 |
| SIGLEC1, ISG15, HERC6, MMP8 | | | | | | | | | |
| RSAD2, OAS1, IFIT1, IFI44L, ISG15, | 0.856 | 0.845 | 0.867 | 0.916 | 0.862 | 0.969 | 0.915 | 0.831 | 0.999 |
| HERC6, SLC1A2, OLAH, FAM20A | | | | | | | | | |
| RSAD2, IFI44L, ISG15, HERC6, | 0.856 | 0.845 | 0.867 | 0.918 | 0.867 | 0.969 | 0.925 | 0.842 | 1 |
| SLC1A2, IL1R2, OLAH, FAM20A, MMP8 | | | | | | | | | |
| RSAD2, OAS1, IFIT1, IFI44L, ISG15, | 0.856 | 0.846 | 0.866 | 0.912 | 0.854 | 0.970 | 0.932 | 0.849 | 1 |
| HERC6, IL1R2, FAM20A, MMP8 | | | | | | | | | |
| RSAD2, OAS1, IFIT1, SIGLEC1, HERC6, | 0.856 | 0.846 | 0.866 | 0.905 | 0.843 | 0.967 | 0.964 | 0.927 | 1 |
| IL1R2, OLAH, RETN, MMP8 | | | | | | | | | |
| RSAD2, IFI27, IFIT1, IFI44L, ISG15, | 0.856 | 0.846 | 0.865 | 0.916 | 0.860 | 0.973 | 0.938 | 0.872 | 1 |
| HERC6, SLC1A2, RETN, MMP8 | | | | | | | | | |
| OAS1, IFI44L, ISG15, HERC6, IL1R2, | 0.856 | 0.845 | 0.866 | 0.912 | 0.852 | 0.972 | 0.943 | 0.871 | 1 |
| OLAH, FAM20A, RETN, MMP8 | | | | | | | | | |
| RSAD2, OAS1, IFI44L, HERC6, | 0.856 | 0.845 | 0.866 | 0.916 | 0.862 | 0.971 | 0.928 | 0.849 | 1 |
| SLC1A2, OLAH, FAM20A, RETN, MMP8 | | | | | | | | | |
| RSAD2, OAS1, IFIT1, SIGLEC1, ISG15, | 0.856 | 0.846 | 0.866 | 0.914 | 0.856 | 0.972 | 0.952 | 0.884 | 1 |
| SLC1A2, FAM20A, RETN, MMP8 | | | | | | | | | |
| IFIT1, IFI44L, SIGLEC1, ISG15, HERC6, | 0.856 | 0.846 | 0.865 | 0.904 | 0.842 | 0.966 | 0.964 | 0.926 | 1 |
| IL1R2, OLAH, RETN, MMP8 | | | | | | | | | |
| RSAD2, OAS1, IFI44L, ISG15, HERC6, | 0.856 | 0.844 | 0.867 | 0.913 | 0.860 | 0.967 | 0.923 | 0.837 | 1 |
| SLC1A2, IL1R2, OLAH, FAM20A | | | | | | | | | |
| RSAD2, OAS1, IFIT1, IFI44L, SIGLEC1, | 0.856 | 0.846 | 0.866 | 0.914 | 0.856 | 0.973 | 0.936 | 0.863 | 1 |
| ISG15, SLC1A2, FAM20A, RETN | | | | | | | | | |
| RSAD2, IFI27, OAS1, IFIT1, IFI44L, | 0.856 | 0.845 | 0.867 | 0.913 | 0.855 | 0.971 | 0.918 | 0.833 | 1 |
| ISG15, HERC6, SLC1A2, FAM20A | | | | | | | | | |
| RSAD2, IFI27, OAS1, IFIT1, IFI44L, | 0.856 | 0.845 | 0.866 | 0.911 | 0.850 | 0.973 | 0.940 | 0.861 | 1 |
| ISG15, SLC1A2, FAM20A, RETN | | | | | | | | | |
| RSAD2, OAS1, IFI44L, ISG15, HERC6, | 0.856 | 0.845 | 0.866 | 0.914 | 0.855 | 0.972 | 0.934 | 0.854 | 1 |
| IL1R2, FAM20A, RETN, MMP8 | | | | | | | | | |
| RSAD2, OAS1, IFIT1, IFI44L, HERC6, | 0.856 | 0.845 | 0.866 | 0.916 | 0.864 | 0.969 | 0.917 | 0.830 | 1 |
| SLC1A2, IL1R2, OLAH, FAM20A | | | | | | | | | |
| RSAD2, OAS1, IFI44L, SIGLEC1, ISG15, | 0.856 | 0.845 | 0.866 | 0.913 | 0.856 | 0.970 | 0.941 | 0.870 | 1 |
| HERC6, SLC1A2, FAM20A, RETN | | | | | | | | | |
| OAS1, IFIT1, HERC6, SLC1A2, IL1R2, | 0.855 | 0.845 | 0.866 | 0.915 | 0.862 | 0.968 | 0.933 | 0.851 | 1 |
| OLAH, FAM20A, RETN, MMP8 | | | | | | | | | |
| OAS1, IFIT1, IFI44L, ISG15, HERC6, | 0.855 | 0.845 | 0.865 | 0.909 | 0.847 | 0.971 | 0.940 | 0.874 | 1 |
| OLAH, FAM20A, RETN, MMP8 | | | | | | | | | |
| OAS1, SIGLEC1, ISG15, HERC6, | 0.855 | 0.845 | 0.865 | 0.916 | 0.862 | 0.969 | 0.963 | 0.925 | 1 |
| SLC1A2, IL1R2, OLAH, RETN, MMP8 | | | | | | | | | |
| OAS1, IFIT1, IFI44L, SIGLEC1, ISG15, | 0.855 | 0.845 | 0.865 | 0.916 | 0.863 | 0.970 | 0.938 | 0.883 | 0.993 |
| HERC6, SLC1A2, OLAH, RETN | | | | | | | | | |
| RSAD2, OAS1, IFIT1, IFI44L, SIGLEC1, | 0.855 | 0.846 | 0.865 | 0.905 | 0.843 | 0.966 | 0.958 | 0.915 | 1 |
| ISG15, HERC6, IL1R2, RETN | | | | | | | | | |
| RSAD2, OAS1, IFIT1, SIGLEC1, HERC6, | 0.855 | 0.845 | 0.865 | 0.914 | 0.859 | 0.969 | 0.952 | 0.905 | 1.000 |
| SLC1A2, OLAH, RETN, MMP8 | | | | | | | | | |
| OAS1, IFIT1, IFI44L, SIGLEC1, ISG15, | 0.855 | 0.845 | 0.865 | 0.904 | 0.842 | 0.967 | 0.955 | 0.912 | 0.999 |
| HERC6, OLAH, RETN, MMP8 | | | | | | | | | |
| IFIT1, SIGLEC1, ISG15, HERC6, | 0.855 | 0.845 | 0.865 | 0.916 | 0.862 | 0.970 | 0.962 | 0.924 | 1 |
| SLC1A2, IL1R2, OLAH, RETN, MMP8 | | | | | | | | | |
| RSAD2, OAS1, IFIT1, IFI44L, HERC6, | 0.855 | 0.845 | 0.865 | 0.908 | 0.846 | 0.971 | 0.936 | 0.866 | 1 |
| OLAH, FAM20A, RETN, MMP8 | | | | | | | | | |
| RSAD2, IFIT1, IFI44L, ISG15, HERC6, | 0.855 | 0.845 | 0.866 | 0.917 | 0.865 | 0.969 | 0.920 | 0.834 | 1 |
| SLC1A2, IL1R2, OLAH, FAM20A | | | | | | | | | |
| RSAD2, IFI44L, ISG15, HERC6, IL1R2, | 0.855 | 0.845 | 0.866 | 0.915 | 0.856 | 0.973 | 0.935 | 0.860 | 1 |
| OLAH, FAM20A, RETN, MMP8 | | | | | | | | | |
| RSAD2, OAS1, IFI44L, ISG15, HERC6, | 0.855 | 0.844 | 0.866 | 0.916 | 0.862 | 0.970 | 0.920 | 0.840 | 0.999 |
| SLC1A2, OLAH, FAM20A, MMP8 | | | | | | | | | |
| RSAD2, IFI27, OAS1, IFI44L, ISG15, | 0.855 | 0.844 | 0.867 | 0.913 | 0.856 | 0.970 | 0.922 | 0.838 | 1 |
| HERC6, SLC1A2, FAM20A, MMP8 | | | | | | | | | |
| RSAD2, IFIT1, ISG15, HERC6, IL1R2, | 0.855 | 0.845 | 0.865 | 0.91C | 0.850 | 0.970 | 0.943 | 0.873 | 1 |
| OLAH, FAM20A, RETN, MMP8 | | | | | | | | | |
| OAS1, IFIT1, IFI44L, ISG15, HERC6, | 0.855 | 0.845 | 0.865 | 0.911 | 0.851 | 0.970 | 0.936 | 0.856 | 1 |
| IL1R2, FAM20A, RETN, MMP8 | | | | | | | | | |
| OAS1, IFIT1, SIGLEC1, ISG15, HERC6, | 0.855 | 0.845 | 0.865 | 0.912 | 0.858 | 0.966 | 0.963 | 0.924 | 1 |
| SLC1A2, IL1R2, RETN, MMP8 | | | | | | | | | |
| RSAD2, OAS1, IFI44L, ISG15, SLC1A2, | 0.855 | 0.845 | 0.866 | 0.918 | 0.867 | 0.969 | 0.924 | 0.841 | 1 |
| IL1R2, OLAH, FAM20A, MMP8 | | | | | | | | | |
| RSAD2, IFIT1, IFI44L, ISG15, HERC6, | 0.855 | 0.844 | 0.866 | 0.913 | 0.855 | 0.970 | 0.932 | 0.853 | 1 |
| IL1R2, OLAH, FAM20A, MMP8 | | | | | | | | | |
| IFIT1, IFI44L, ISG15, HERC6, SLC1A2, | 0.855 | 0.845 | 0.865 | 0.915 | 0.862 | 0.968 | 0.921 | 0.835 | 1 |
| IL1R2, OLAH, FAM20A, RETN | | | | | | | | | |
| IFIT1, IFI44L, ISG15, SLC1A2, IL1R2, | 0.855 | 0.845 | 0.865 | 0.915 | 0.862 | 0.969 | 0.932 | 0.853 | 1 |
| OLAH, FAM20A, RETN, MMP8 | | | | | | | | | |
| OAS1, IFI44L, HERC6, SLC1A2, IL1R2, | 0.855 | 0.844 | 0.866 | 0.919 | 0.867 | 0.971 | 0.929 | 0.846 | 1 |
| OLAH, FAM20A, RETN, MMP8 | | | | | | | | | |
| RSAD2, OAS1, HERC6, SLC1A2, IL1R2, | 0.855 | 0.845 | 0.865 | 0.914 | 0.861 | 0.967 | 0.929 | 0.843 | 1 |
| OLAH, FAM20A, RETN, MMP8 | | | | | | | | | |
| RSAD2, OAS1, SIGLEC1, HERC6, | 0.855 | 0.845 | 0.865 | 0.914 | 0.859 | 0.969 | 0.966 | 0.930 | 1 |
| SLC1A2, IL1R2, OLAH, RETN, MMP8 | | | | | | | | | |
| IFI27, OAS1, IFIT1, IFI44L, ISG15, | 0.855 | 0.844 | 0.866 | 0.913 | 0.852 | 0.974 | 0.938 | 0.858 | 1 |
| SLC1A2, FAM20A, RETN, MMP8 | | | | | | | | | |
| RSAD2, OAS1, IFIT1, IFI44L, HERC6, | 0.855 | 0.845 | 0.865 | 0.911 | 0.851 | 0.971 | 0.940 | 0.863 | 1 |
| IL1R2, FAM20A, RETN, MMP8 | | | | | | | | | |
| IFIT1, ISG15, HERC6, SLC1A2, IL1R2, | 0.855 | 0.845 | 0.865 | 0.914 | 0.861 | 0.968 | 0.934 | 0.853 | 1 |
| OLAH, FAM20A, RETN, MMP8 | | | | | | | | | |
| RSAD2, OAS1, SIGLEC1, ISG15, | 0.855 | 0.844 | 0.866 | 0.907 | 0.846 | 0.968 | 0.968 | 0.934 | 1 |
| HERC6, IL1R2, OLAH, RETN, MMP8 | | | | | | | | | |
| RSAD2, IFIT1, IFI44L, ISG15, HERC6, | 0.855 | 0.845 | 0.865 | 0.912 | 0.852 | 0.971 | 0.936 | 0.867 | 1 |
| OLAH, FAM20A, RETN, MMP8 | | | | | | | | | |
| IFIT1, IFI44L, SIGLEC1, ISG15, HERC6, | 0.855 | 0.845 | 0.865 | 0.914 | 0.859 | 0.969 | 0.950 | 0.904 | 0.996 |
| SLC1A2, OLAH, RETN, MMP8 | | | | | | | | | |
| IFI27, OAS1, IFIT1, IFI44L, SIGLEC1, | 0.855 | 0.846 | 0.864 | 0.913 | 0.861 | 0.965 | 0.915 | 0.843 | 0.987 |
| ISG15, HERC6, SLC1A2, MMP8 | | | | | | | | | |
| RSAD2, IFIT1, ISG15, HERC6, SLC1A2, | 0.855 | 0.845 | 0.865 | 0.916 | 0.864 | 0.967 | 0.932 | 0.850 | 1 |
| IL1R2, OLAH, FAM20A, MMP8 | | | | | | | | | |
| OAS1, IFIT1, ISG15, HERC6, SLC1A2, | 0.855 | 0.844 | 0.865 | 0.916 | 0.865 | 0.968 | 0.933 | 0.852 | 1 |
| IL1R2, OLAH, FAM20A, MMP8 | | | | | | | | | |
| RSAD2, IFI27, IFIT1, IFI44L, ISG15, | 0.855 | 0.844 | 0.866 | 0.913 | 0.851 | 0.974 | 0.939 | 0.860 | 1 |
| SLC1A2, FAM20A, RETN, MMP8 | | | | | | | | | |
| RSAD2, OAS1, IFIT1, IFI44L, SIGLEC1, | 0.855 | 0.845 | 0.865 | 0.916 | 0.861 | 0.971 | 0.941 | 0.886 | 0.996 |
| HERC6, SLC1A2, OLAH, RETN | | | | | | | | | |
| RSAD2, IFIT1, IFI44L, ISG15, HERC6, | 0.855 | 0.845 | 0.865 | 0.914 | 0.855 | 0.972 | 0.932 | 0.852 | 1 |
| IL1R2, FAM20A, RETN, MMP8 | | | | | | | | | |
| RSAD2, IFIT1, IFI44L, ISG15, SLC1A2, | 0.855 | 0.845 | 0.864 | 0.915 | 0.863 | 0.968 | 0.929 | 0.849 | 1 |
| IL1R2, OLAH, FAM20A, MMP8 | | | | | | | | | |
| RSAD2, OAS1, IFI44L, SIGLEC1, ISG15, | 0.855 | 0.844 | 0.865 | 0.911 | 0.854 | 0.968 | 0.938 | 0.863 | 1 |
| HERC6, SLC1A2, FAM20A, MMP8 | | | | | | | | | |
| RSAD2, IFIT1, SIGLEC1, ISG15, | 0.855 | 0.845 | 0.865 | 0.906 | 0.845 | 0.967 | 0.964 | 0.926 | 1 |
| HERC6, IL1R2, OLAH, RETN, MMP8 | | | | | | | | | |
| RSAD2, OAS1, ISG15, HERC6, SLC1A2, | 0.855 | 0.844 | 0.865 | 0.914 | 0.861 | 0.966 | 0.927 | 0.840 | 1 |
| IL1R2, OLAH, FAM20A, MMP8 | | | | | | | | | |
| RSAD2, OAS1, IFIT1, ISG15, HERC6, | 0.855 | 0.844 | 0.865 | 0.906 | 0.845 | 0.966 | 0.937 | 0.862 | 1 |
| IL1R2, OLAH, FAM20A, RETN | | | | | | | | | |
| OAS1, IFIT1, IFI44L, ISG15, HERC6, | 0.855 | 0.845 | 0.864 | 0.907 | 0.848 | 0.967 | 0.932 | 0.854 | 1 |
| IL1R2, OLAH, FAM20A, RETN | | | | | | | | | |
| OAS1, ISG15, HERC6, SLC1A2, IL1R2, | 0.854 | 0.844 | 0.865 | 0.916 | 0.863 | 0.969 | 0.929 | 0.844 | 1 |
| OLAH, FAM20A, RETN, MMP8 | | | | | | | | | |
| RSAD2, OAS1, IFI44L, ISG15, HERC6, | 0.854 | 0.844 | 0.865 | 0.910 | 0.851 | 0.969 | 0.937 | 0.864 | 1 |
| IL1R2, OLAH, FAM20A, RETN | | | | | | | | | |
| OAS1, IFIT1, SIGLEC1, ISG15, HERC6, | 0.854 | 0.844 | 0.865 | 0.914 | 0.860 | 0.968 | 0.950 | 0.903 | 0.997 |
| SLC1A2, IL1R2, OLAH, RETN | | | | | | | | | |
| RSAD2, OAS1, IFIT1, IFI44L, ISG15, | 0.854 | 0.844 | 0.865 | 0.908 | 0.847 | 0.969 | 0.945 | 0.876 | 1 |
| IL1R2, OLAH, FAM20A, MMP8 | | | | | | | | | |
| RSAD2, OAS1, IFIT1, SIGLEC1, ISG15, | 0.854 | 0.844 | 0.864 | 0.907 | 0.846 | 0.967 | 0.958 | 0.915 | 1 |
| HERC6, IL1R2, OLAH, RETN | | | | | | | | | |
| RSAD2, OAS1, IFIT1, IFI44L, ISG15, | 0.854 | 0.844 | 0.865 | 0.915 | 0.859 | 0.971 | 0.922 | 0.844 | 0.999 |
| SLC1A2, OLAH, FAM20A, MMP8 | | | | | | | | | |
| OAS1, IFIT1, IFI44L, ISG15, SLC1A2, | 0.854 | 0.844 | 0.864 | 0.916 | 0.864 | 0.968 | 0.927 | 0.845 | 1 |
| IL1R2, OLAH, FAM20A, MMP8 | | | | | | | | | |
| RSAD2, OAS1, IFIT1, SIGLEC1, HERC6, | 0.854 | 0.844 | 0.865 | 0.915 | 0.860 | 0.969 | 0.950 | 0.900 | 1.000 |
| SLC1A2, IL1R2, OLAH, RETN | | | | | | | | | |
| RSAD2, IFIT1, IFI44L, ISG15, HERC6, | 0.854 | 0.844 | 0.864 | 0.917 | 0.864 | 0.970 | 0.916 | 0.831 | 1 |
| SLC1A2, OLAH, FAM20A, RETN | | | | | | | | | |
| RSAD2, IFIT1, SIGLEC1, HERC6, | 0.854 | 0.844 | 0.864 | 0.916 | 0.862 | 0.970 | 0.961 | 0.922 | 1.000 |
| SLC1A2, IL1R2, OLAH, RETN, MMP8 | | | | | | | | | |
| RSAD2, OAS1, IFIT1, IFI44L, HERC6, | 0.854 | 0.844 | 0.864 | 0.918 | 0.868 | 0.969 | 0.925 | 0.832 | 1 |
| SLC1A2, IL1R2, FAM20A, MMP8 | | | | | | | | | |
| RSAD2, OAS1, IFIT1, SIGLEC1, ISG15, | 0.854 | 0.844 | 0.864 | 0.912 | 0.858 | 0.966 | 0.962 | 0.923 | 1 |
| HERC6, SLC1A2, IL1R2, OLAH | | | | | | | | | |
| RSAD2, IFIT1, IFI44L, SIGLEC1, ISG15, | 0.854 | 0.844 | 0.864 | 0.904 | 0.842 | 0.966 | 0.964 | 0.926 | 1 |
| HERC6, IL1R2, RETN, MMP8 | | | | | | | | | |
| RSAD2, IFI44L, ISG15, HERC6, | 0.854 | 0.843 | 0.865 | 0.918 | 0.863 | 0.972 | 0.927 | 0.848 | 1 |
| SLC1A2, OLAH, FAM20A, RETN, MMP8 | | | | | | | | | |
| RSAD2, ISG15, HERC6, SLC1A2, IL1R2, | 0.854 | 0.844 | 0.864 | 0.916 | 0.864 | 0.968 | 0.929 | 0.843 | 1 |
| OLAH, FAM20A, RETN, MMP8 | | | | | | | | | |
| OAS1, IFIT1, SIGLEC1, ISG15, HERC6, | 0.854 | 0.844 | 0.864 | 0.913 | 0.860 | 0.966 | 0.962 | 0.924 | 1 |
| SLC1A2, IL1R2, OLAH, MMP8 | | | | | | | | | |
| RSAD2, OAS1, IFIT1, IFI44L, ISG15, | 0.854 | 0.844 | 0.864 | 0.913 | 0.855 | 0.970 | 0.934 | 0.855 | 1 |
| HERC6, IL1R2, FAM20A, RETN | | | | | | | | | |
| RSAD2, OAS1, IFIT1, IFI44L, ISG15, | 0.854 | 0.843 | 0.865 | 0.907 | 0.846 | 0.967 | 0.933 | 0.863 | 1 |
| HERC6, OLAH, FAM20A, MMP8 | | | | | | | | | |
| RSAD2, IFIT1, IFI44L, SIGLEC1, | 0.854 | 0.844 | 0.864 | 0.904 | 0.841 | 0.966 | 0.964 | 0.926 | 1 |
| HERC6, IL1R2, OLAH, RETN, MMP8 | | | | | | | | | |
| RSAD2, OAS1, IFIT1, SIGLEC1, ISG15, | 0.854 | 0.844 | 0.864 | 0.911 | 0.856 | 0.965 | 0.957 | 0.909 | 1 |
| HERC6, SLC1A2, IL1R2, RETN | | | | | | | | | |
| RSAD2, OAS1, IFIT1, IFI44L, ISG15, | 0.854 | 0.844 | 0.864 | 0.908 | 0.849 | 0.968 | 0.933 | 0.863 | 1 |
| HERC6, OLAH, FAM20A, RETN | | | | | | | | | |
| OAS1, IFIT1, ISG15, HERC6, SLC1A2, | 0.854 | 0.844 | 0.864 | 0.914 | 0.860 | 0.968 | 0.930 | 0.844 | 1 |
| IL1R2, FAM20A, RETN, MMP8 | | | | | | | | | |
| OAS1, IFIT1, IFI44L, SIGLEC1, ISG15, | 0.854 | 0.844 | 0.864 | 0.907 | 0.847 | 0.967 | 0.952 | 0.907 | 0.998 |
| HERC6, IL1R2, OLAH, RETN | | | | | | | | | |
| RSAD2, IFIT1, IFI44L, HERC6, IL1R2, | 0.854 | 0.844 | 0.864 | 0.912 | 0.853 | 0.972 | 0.941 | 0.868 | 1 |
| OLAH, FAM20A, RETN, MMP8 | | | | | | | | | |
| OAS1, IFIT1, IFI44L, SIGLEC1, HERC6, | 0.854 | 0.844 | 0.864 | 0.911 | 0.856 | 0.966 | 0.961 | 0.921 | 1 |
| SLC1A2, IL1R2, RETN, MMP8 | | | | | | | | | |
| RSAD2, OAS1, IFIT1, SIGLEC1, HERC6, | 0.854 | 0.844 | 0.864 | 0.913 | 0.859 | 0.968 | 0.965 | 0.927 | 1 |
| SLC1A2, IL1R2, RETN, MMP8 | | | | | | | | | |
| RSAD2, IFI27, OAS1, IFI44L, ISG15, | 0.854 | 0.842 | 0.865 | 0.913 | 0.851 | 0.974 | 0.940 | 0.862 | 1 |
| SLC1A2, FAM20A, RETN, MMP8 | | | | | | | | | |
| IFIT1, IFI44L, SIGLEC1, HERC6, | 0.854 | 0.844 | 0.864 | 0.915 | 0.861 | 0.969 | 0.959 | 0.918 | 0.999 |
| SLC1A2, IL1R2, OLAH, RETN, MMP8 | | | | | | | | | |
| RSAD2, OAS1, IFIT1, IFI44L, HERC6, | 0.854 | 0.844 | 0.864 | 0.917 | 0.864 | 0.970 | 0.913 | 0.826 | 1 |
| SLC1A2, OLAH, FAM20A, RETN | | | | | | | | | |
| RSAD2, IFI27, OAS1, IFIT1, IFI44L, | 0.854 | 0.843 | 0.865 | 0.912 | 0.851 | 0.974 | 0.940 | 0.861 | 1 |
| SLC1A2, FAM20A, RETN, MMP8 | | | | | | | | | |
| OAS1, IFI44L, ISG15, HERC6, SLC1A2, | 0.854 | 0.842 | 0.865 | 0.911 | 0.856 | 0.966 | 0.924 | 0.839 | 1 |
| IL1R2, OLAH, FAM20A, RETN | | | | | | | | | |
| RSAD2, OAS1, IFIT1, SIGLEC1, ISG15, | 0.854 | 0.844 | 0.864 | 0.915 | 0.860 | 0.970 | 0.950 | 0.897 | 1 |
| HERC6, SLC1A2, OLAH, RETN | | | | | | | | | |
| RSAD2, IFI27, IFIT1, IFI44L, SIGLEC1, | 0.854 | 0.844 | 0.863 | 0.915 | 0.859 | 0.971 | 0.951 | 0.892 | 1 |
| ISG15, SLC1A2, RETN, MMP8 | | | | | | | | | |
| RSAD2, OAS1, ISG15, HERC6, SLC1A2, | 0.854 | 0.844 | 0.864 | 0.917 | 0.864 | 0.969 | 0.928 | 0.839 | 1 |
| IL1R2, FAM20A, RETN, MMP8 | | | | | | | | | |
| RSAD2, OAS1, IFIT1, SIGLEC1, HERC6, | 0.854 | 0.843 | 0.864 | 0.912 | 0.858 | 0.966 | 0.963 | 0.925 | 1 |
| SLC1A2, IL1R2, OLAH, MMP8 | | | | | | | | | |
| RSAD2, OAS1, ISG15, HERC6, SLC1A2, | 0.854 | 0.843 | 0.864 | 0.912 | 0.858 | 0.967 | 0.928 | 0.843 | 1 |
| IL1R2, OLAH, FAM20A, RETN | | | | | | | | | |
| RSAD2, OAS1, IFIT1, IFI44L, SIGLEC1, | 0.854 | 0.843 | 0.864 | 0.901 | 0.836 | 0.966 | 0.953 | 0.909 | 0.997 |
| HERC6, OLAH, RETN, MMP8 | | | | | | | | | |
| RSAD2, OAS1, IFIT1, IFI44L, SIGLEC1, | 0.853 | 0.844 | 0.863 | 0.905 | 0.842 | 0.968 | 0.955 | 0.911 | 0.999 |
| HERC6, IL1R2, OLAH, RETN | | | | | | | | | |
| OAS1, IFIT1, ISG15, HERC6, SLC1A2, | 0.853 | 0.843 | 0.864 | 0.910 | 0.852 | 0.968 | 0.936 | 0.859 | 1 |
| OLAH, FAM20A, RETN, MMP8 | | | | | | | | | |
| OAS1, IFIT1, IFI44L, HERC6, SLC1A2, | 0.853 | 0.843 | 0.864 | 0.918 | 0.866 | 0.969 | 0.917 | 0.831 | 1 |
| IL1R2, OLAH, FAM20A, RETN | | | | | | | | | |
| RSAD2, OAS1, IFIT1, IFI44L, SIGLEC1, | 0.853 | 0.843 | 0.864 | 0.912 | 0.857 | 0.967 | 0.951 | 0.905 | 0.998 |
| HERC6, SLC1A2, OLAH, MMP8 | | | | | | | | | |
| RSAD2, IFI27, OAS1, IFI44L, SIGLEC1, | 0.853 | 0.844 | 0.863 | 0.913 | 0.860 | 0.967 | 0.925 | 0.854 | 0.996 |
| ISG15, HERC6, SLC1A2, MMP8 | | | | | | | | | |
| RSAD2, OAS1, IFIT1, SIGLEC1, ISG15, | 0.853 | 0.843 | 0.864 | 0.905 | 0.843 | 0.967 | 0.958 | 0.916 | 1.000 |
| HERC6, OLAH, RETN, MMP8 | | | | | | | | | |
| RSAD2, IFIT1, SIGLEC1, ISG15, | 0.853 | 0.844 | 0.863 | 0.914 | 0.860 | 0.967 | 0.962 | 0.924 | 1 |
| HERC6, SLC1A2, IL1R2, OLAH, MMP8 | | | | | | | | | |
| OAS1, IFI44L, SIGLEC1, ISG15, HERC6, | 0.853 | 0.843 | 0.864 | 0.906 | 0.845 | 0.967 | 0.967 | 0.932 | 1 |
| IL1R2, OLAH, RETN, MMP8 | | | | | | | | | |
| OAS1, IFIT1, IFI44L, SIGLEC1, ISG15, | 0.853 | 0.843 | 0.864 | 0.913 | 0.858 | 0.968 | 0.945 | 0.894 | 0.995 |
| HERC6, SLC1A2, OLAH, MMP8 | | | | | | | | | |
| IFIT1, IFI44L, ISG15, HERC6, SLC1A2, | 0.853 | 0.844 | 0.863 | 0.916 | 0.863 | 0.969 | 0.928 | 0.837 | 1 |
| IL1R2, FAM20A, RETN, MMP8 | | | | | | | | | |
| OAS1, IFIT1, IFI44L, HERC6, SLC1A2, | 0.853 | 0.843 | 0.863 | 0.915 | 0.862 | 0.969 | 0.927 | 0.836 | 1 |
| IL1R2, FAM20A, RETN, MMP8 | | | | | | | | | |
| RSAD2, OAS1, SIGLEC1, ISG15, | 0.853 | 0.843 | 0.863 | 0.911 | 0.857 | 0.964 | 0.962 | 0.923 | 1 |
| HERC6, SLC1A2, IL1R2, OLAH, MMP8 | | | | | | | | | |
| OAS1, IFIT1, IFI44L, SIGLEC1, ISG15, | 0.853 | 0.843 | 0.863 | 0.913 | 0.860 | 0.966 | 0.952 | 0.904 | 1.000 |
| HERC6, SLC1A2, IL1R2, RETN | | | | | | | | | |
| RSAD2, OAS1, IFIT1, HERC6, SLC1A2, | 0.853 | 0.843 | 0.863 | 0.914 | 0.859 | 0.969 | 0.934 | 0.850 | 1 |
| IL1R2, FAM20A, RETN, MMP8 | | | | | | | | | |
| RSAD2, IFIT1, IFI44L, ISG15, HERC6, | 0.853 | 0.843 | 0.863 | 0.909 | 0.850 | 0.968 | 0.932 | 0.855 | 1 |
| IL1R2, OLAH, FAM20A, RETN | | | | | | | | | |
| OAS1, IFI44L, SIGLEC1, ISG15, HERC6, | 0.853 | 0.843 | 0.863 | 0.912 | 0.855 | 0.969 | 0.962 | 0.921 | 1 |
| SLC1A2, OLAH, RETN, MMP8 | | | | | | | | | |
| RSAD2, OAS1, IFIT1, ISG15, SLC1A2, | 0.853 | 0.843 | 0.863 | 0.917 | 0.865 | 0.969 | 0.936 | 0.859 | 1 |
| IL1R2, OLAH, FAM20A, MMP8 | | | | | | | | | |
| RSAD2, OAS1, IFIT1, IFI44L, ISG15, | 0.853 | 0.843 | 0.863 | 0.915 | 0.865 | 0.966 | 0.925 | 0.834 | 1 |
| HERC6, SLC1A2, IL1R2, FAM20A | | | | | | | | | |
| RSAD2, OAS1, IFIT1, IFI44L, SIGLEC1, | 0.853 | 0.843 | 0.863 | 0.904 | 0.845 | 0.963 | 0.963 | 0.924 | 1 |
| HERC6, IL1R2, OLAH, MMP8 | | | | | | | | | |
| RSAD2, OAS1, IFI44L, SIGLEC1, | 0.853 | 0.842 | 0.864 | 0.901 | 0.838 | 0.965 | 0.965 | 0.927 | 1 |
| HERC6, IL1R2, OLAH, RETN, MMP8 | | | | | | | | | |
| RSAD2, OAS1, IFI44L, ISG15, HERC6, | 0.853 | 0.842 | 0.864 | 0.912 | 0.851 | 0.972 | 0.938 | 0.872 | 1 |
| OLAH, FAM20A, RETN, MMP8 | | | | | | | | | |
| OAS1, IFIT1, ISG15, HERC6, SLC1A2, | 0.853 | 0.842 | 0.864 | 0.912 | 0.857 | 0.966 | 0.927 | 0.842 | 1 |
| IL1R2, OLAH, FAM20A, RETN | | | | | | | | | |
| IFIT1, IFI44L, SIGLEC1, ISG15, HERC6, | 0.853 | 0.843 | 0.863 | 0.913 | 0.860 | 0.967 | 0.961 | 0.922 | 1.000 |
| SLC1A2, IL1R2, OLAH, MMP8 | | | | | | | | | |
| RSAD2, OAS1, IFI44L, ISG15, HERC6, | 0.853 | 0.842 | 0.864 | 0.918 | 0.867 | 0.968 | 0.924 | 0.830 | 1 |
| SLC1A2, IL1R2, FAM20A, MMP8 | | | | | | | | | |
| RSAD2, OAS1, SIGLEC1, ISG15, | 0.853 | 0.843 | 0.863 | 0.913 | 0.857 | 0.970 | 0.961 | 0.919 | 1 |
| HERC6, SLC1A2, OLAH, RETN, MMP8 | | | | | | | | | |
| RSAD2, OAS1, IFIT1, IFI44L, SIGLEC1, | 0.853 | 0.843 | 0.862 | 0.905 | 0.847 | 0.964 | 0.958 | 0.915 | 1 |
| ISG15, HERC6, IL1R2, OLAH | | | | | | | | | |
| RSAD2, OAS1, IFIT1, ISG15, HERC6, | 0.853 | 0.842 | 0.863 | 0.912 | 0.858 | 0.966 | 0.928 | 0.844 | 1 |
| SLC1A2, IL1R2, OLAH, FAM20A | | | | | | | | | |
| RSAD2, IFIT1, IFI44L, HERC6, SLC1A2, | 0.853 | 0.843 | 0.863 | 0.917 | 0.865 | 0.969 | 0.917 | 0.831 | 1 |
| IL1R2, OLAH, FAM20A, RETN | | | | | | | | | |
| RSAD2, IFIT1, HERC6, SLC1A2, IL1R2, | 0.853 | 0.842 | 0.863 | 0.915 | 0.863 | 0.968 | 0.933 | 0.851 | 1 |
| OLAH, FAM20A, RETN, MMP8 | | | | | | | | | |
| OAS1, IFI44L, SIGLEC1, HERC6, | 0.853 | 0.843 | 0.863 | 0.915 | 0.860 | 0.969 | 0.962 | 0.923 | 1 |
| SLC1A2, IL1R2, OLAH, RETN, MMP8 | | | | | | | | | |
| RSAD2, OAS1, ISG15, HERC6, SLC1A2, | 0.853 | 0.843 | 0.863 | 0.911 | 0.855 | 0.968 | 0.935 | 0.856 | 1 |
| OLAH, FAM20A, RETN, MMP8 | | | | | | | | | |
| RSAD2, OAS1, IFIT1, IFI44L, HERC6, | 0.853 | 0.843 | 0.863 | 0.907 | 0.846 | 0.968 | 0.934 | 0.858 | 1 |
| IL1R2, OLAH, FAM20A, RETN | | | | | | | | | |
| RSAD2, OAS1, IFIT1, SIGLEC1, ISG15, | 0.853 | 0.842 | 0.863 | 0.912 | 0.857 | 0.967 | 0.951 | 0.905 | 0.998 |
| HERC6, SLC1A2, OLAH, MMP8 | | | | | | | | | |
| RSAD2, OAS1, IFI44L, SIGLEC1, ISG15, | 0.853 | 0.843 | 0.863 | 0.903 | 0.841 | 0.965 | 0.967 | 0.932 | 1 |
| HERC6, IL1R2, RETN, MMP8 | | | | | | | | | |
| RSAD2, SIGLEC1, ISG15, HERC6, | 0.853 | 0.843 | 0.862 | 0.916 | 0.862 | 0.969 | 0.962 | 0.923 | 1 |
| SLC1A2, IL1R2, OLAH, RETN, MMP8 | | | | | | | | | |
| RSAD2, IFI44L, ISG15, SLC1A2, IL1R2, | 0.853 | 0.842 | 0.863 | 0.917 | 0.864 | 0.969 | 0.929 | 0.851 | 1 |
| OLAH, FAM20A, RETN, MMP8 | | | | | | | | | |
| RSAD2, OAS1, IFI44L, SLC1A2, IL1R2, | 0.853 | 0.842 | 0.863 | 0.919 | 0.867 | 0.970 | 0.930 | 0.852 | 1 |
| OLAH, FAM20A, RETN, MMP8 | | | | | | | | | |
| RSAD2, OAS1, SIGLEC1, ISG15, | 0.853 | 0.843 | 0.863 | 0.912 | 0.857 | 0.967 | 0.966 | 0.929 | 1 |
| HERC6, SLC1A2, IL1R2, RETN, MMP8 | | | | | | | | | |
| RSAD2, IFIT1, SIGLEC1, ISG15, | 0.853 | 0.843 | 0.862 | 0.914 | 0.859 | 0.969 | 0.952 | 0.907 | 0.997 |
| HERC6, SLC1A2, OLAH, RETN, MMP8 | | | | | | | | | |
| RSAD2, IFIT1, IFI44L, SIGLEC1, | 0.853 | 0.843 | 0.862 | 0.912 | 0.858 | 0.965 | 0.962 | 0.923 | 1 |
| HERC6, SLC1A2, IL1R2, OLAH, MMP8 | | | | | | | | | |
| RSAD2, IFIT1, ISG15, HERC6, SLC1A2, | 0.853 | 0.843 | 0.862 | 0.915 | 0.861 | 0.969 | 0.930 | 0.844 | 1 |
| IL1R2, FAM20A, RETN, MMP8 | | | | | | | | | |
| RSAD2, IFI44L, ISG15, HERC6, | 0.853 | 0.841 | 0.864 | 0.913 | 0.858 | 0.967 | 0.925 | 0.841 | 1 |
| SLC1A2, IL1R2, OLAH, FAM20A, RETN | | | | | | | | | |
| RSAD2, OAS1, IFIT1, ISG15, HERC6, | 0.853 | 0.842 | 0.863 | 0.905 | 0.843 | 0.967 | 0.943 | 0.876 | 1 |
| OLAH, FAM20A, RETN, MMP8 | | | | | | | | | |
| RSAD2, OAS1, IFIT1, SLC1A2, IL1R2, | 0.853 | 0.843 | 0.862 | 0.913 | 0.858 | 0.968 | 0.937 | 0.861 | 1 |
| OLAH, FAM20A, RETN, MMP8 | | | | | | | | | |
| OAS1, IFIT1, IFI44L, ISG15, HERC6, | 0.853 | 0.842 | 0.863 | 0.917 | 0.866 | 0.967 | 0.925 | 0.832 | 1 |
| SLC1A2, IL1R2, FAM20A, MMP8 | | | | | | | | | |
| RSAD2, IFI27, OAS1, IFIT1, ISG15, | 0.853 | 0.842 | 0.863 | 0.910 | 0.847 | 0.972 | 0.942 | 0.864 | 1 |
| SLC1A2, FAM20A, RETN, MMP8 | | | | | | | | | |
| OAS1, IFI44L, ISG15, SLC1A2, IL1R2, | 0.853 | 0.842 | 0.863 | 0.917 | 0.865 | 0.970 | 0.928 | 0.847 | 1 |
| OLAH, FAM20A, RETN, MMP8 | | | | | | | | | |
| RSAD2, OAS1, IFIT1, IFI44L, SIGLEC1, | 0.853 | 0.843 | 0.862 | 0.914 | 0.860 | 0.967 | 0.962 | 0.923 | 1 |
| HERC6, SLC1A2, IL1R2, OLAH | | | | | | | | | |
| RSAD2, OAS1, SIGLEC1, ISG15, | 0.852 | 0.842 | 0.863 | 0.911 | 0.856 | 0.967 | 0.959 | 0.915 | 1 |
| HERC6, SLC1A2, IL1R2, OLAH, RETN | | | | | | | | | |
| OAS1, IFI44L, ISG15, HERC6, SLC1A2, | 0.852 | 0.842 | 0.863 | 0.915 | 0.862 | 0.968 | 0.926 | 0.836 | 1 |
| IL1R2, FAM20A, RETN, MMP8 | | | | | | | | | |
| RSAD2, OAS1, IFIT1, ISG15, HERC6, | 0.852 | 0.843 | 0.862 | 0.916 | 0.864 | 0.968 | 0.929 | 0.842 | 1 |
| SLC1A2, IL1R2, FAM20A, MMP8 | | | | | | | | | |
| OAS1, IFIT1, IFI44L, SIGLEC1, HERC6, | 0.852 | 0.842 | 0.863 | 0.913 | 0.860 | 0.966 | 0.962 | 0.924 | 1 |
| SLC1A2, IL1R2, OLAH, MMP8 | | | | | | | | | |
| RSAD2, OAS1, IFIT1, IFI44L, SIGLEC1, | 0.852 | 0.842 | 0.863 | 0.912 | 0.857 | 0.967 | 0.953 | 0.908 | 0.999 |
| ISG15, HERC6, SLC1A2, OLAH | | | | | | | | | |
| RSAD2, OAS1, IFIT1, ISG15, IL1R2, | 0.852 | 0.842 | 0.863 | 0.906 | 0.844 | 0.968 | 0.947 | 0.878 | 1 |
| OLAH, FAM20A, RETN, MMP8 | | | | | | | | | |
| RSAD2, IFIT1, IFI44L, SIGLEC1, | 0.852 | 0.843 | 0.862 | 0.914 | 0.859 | 0.969 | 0.945 | 0.895 | 0.994 |
| HERC6, SLC1A2, OLAH, RETN, MMP8 | | | | | | | | | |
| RSAD2, IFIT1, IFI44L, SIGLEC1, ISG15, | 0.852 | 0.842 | 0.862 | 0.912 | 0.858 | 0.967 | 0.941 | 0.889 | 0.993 |
| HERC6, SLC1A2, OLAH, MMP8 | | | | | | | | | |
| OAS1, IFIT1, ISG15, SLC1A2, IL1R2, | 0.852 | 0.842 | 0.862 | 0.915 | 0.861 | 0.969 | 0.937 | 0.861 | 1 |
| OLAH, FAM20A, RETN, MMP8 | | | | | | | | | |
| OAS1, IFIT1, IFI44L, SIGLEC1, ISG15, | 0.852 | 0.842 | 0.862 | 0.913 | 0.859 | 0.967 | 0.954 | 0.911 | 0.998 |
| HERC6, SLC1A2, IL1R2, OLAH | | | | | | | | | |
| RSAD2, OAS1, IFI44L, HERC6, | 0.852 | 0.841 | 0.863 | 0.914 | 0.860 | 0.967 | 0.923 | 0.838 | 1 |
| SLC1A2, IL1R2, OLAH, FAM20A, RETN | | | | | | | | | |
| RSAD2, OAS1, IFI44L, SIGLEC1, ISG15, | 0.852 | 0.842 | 0.863 | 0.911 | 0.851 | 0.970 | 0.949 | 0.878 | 1 |
| SLC1A2, FAM20A, RETN, MMP8 | | | | | | | | | |
| OAS1, IFIT1, IFI44L, ISG15, SLC1A2, | 0.852 | 0.842 | 0.863 | 0.914 | 0.857 | 0.970 | 0.922 | 0.845 | 0.999 |
| OLAH, FAM20A, RETN, MMP8 | | | | | | | | | |
| RSAD2, IFIT1, IFI44L, SLC1A2, IL1R2, | 0.852 | 0.842 | 0.862 | 0.916 | 0.862 | 0.969 | 0.934 | 0.858 | 1 |
| OLAH, FAM20A, RETN, MMP8 | | | | | | | | | |
| OAS1, IFIT1, IFI44L, SIGLEC1, HERC6, | 0.852 | 0.842 | 0.863 | 0.915 | 0.862 | 0.968 | 0.945 | 0.894 | 0.995 |
| SLC1A2, IL1R2, OLAH, RETN | | | | | | | | | |
| RSAD2, OAS1, IFIT1, IFI44L, SIGLEC1, | 0.852 | 0.842 | 0.862 | 0.905 | 0.843 | 0.968 | 0.947 | 0.897 | 0.997 |
| ISG15, HERC6, OLAH, RETN | | | | | | | | | |
| RSAD2, IFI44L, ISG15, HERC6, | 0.852 | 0.842 | 0.862 | 0.917 | 0.864 | 0.970 | 0.927 | 0.838 | 1 |
| SLC1A2, IL1R2, FAM20A, RETN, MMP8 | | | | | | | | | |
| OAS1, IFIT1, IFI44L, SIGLEC1, ISG15, | 0.852 | 0.842 | 0.862 | 0.906 | 0.849 | 0.964 | 0.963 | 0.924 | 1 |
| HERC6, IL1R2, OLAH, MMP8 | | | | | | | | | |
| IFIT1, IFI44L, SIGLEC1, ISG15, HERC6, | 0.852 | 0.843 | 0.861 | 0.911 | 0.857 | 0.966 | 0.966 | 0.931 | 1 |
| SLC1A2, IL1R2, RETN, MMP8 | | | | | | | | | |
| RSAD2, OAS1, IFI44L, ISG15, HERC6, | 0.852 | 0.841 | 0.863 | 0.914 | 0.857 | 0.970 | 0.925 | 0.846 | 1 |
| SLC1A2, OLAH, FAM20A, RETN | | | | | | | | | |
| RSAD2, OAS1, IFIT1, IFI44L, SIGLEC1, | 0.852 | 0.842 | 0.861 | 0.904 | 0.845 | 0.962 | 0.960 | 0.919 | 1 |
| ISG15, HERC6, IL1R2, MMP8 | | | | | | | | | |
| OAS1, IFIT1, SIGLEC1, ISG15, SLC1A2, | 0.852 | 0.842 | 0.862 | 0.913 | 0.859 | 0.967 | 0.966 | 0.931 | 1 |
| IL1R2, OLAH, RETN, MMP8 | | | | | | | | | |
| OAS1, IFIT1, IFI44L, SLC1A2, IL1R2, | 0.852 | 0.842 | 0.862 | 0.916 | 0.863 | 0.969 | 0.934 | 0.857 | 1 |
| OLAH, FAM20A, RETN, MMP8 | | | | | | | | | |
| RSAD2, IFIT1, SIGLEC1, ISG15, | 0.852 | 0.842 | 0.862 | 0.914 | 0.859 | 0.969 | 0.951 | 0.905 | 0.997 |
| HERC6, SLC1A2, IL1R2, OLAH, RETN | | | | | | | | | |
| RSAD2, OAS1, IFI44L, SIGLEC1, | 0.852 | 0.842 | 0.862 | 0.912 | 0.858 | 0.966 | 0.962 | 0.923 | 1 |
| HERC6, SLC1A2, IL1R2, OLAH, MMP8 | | | | | | | | | |
| IFIT1, IFI44L, SIGLEC1, ISG15, HERC6, | 0.852 | 0.842 | 0.862 | 0.913 | 0.858 | 0.968 | 0.949 | 0.902 | 0.996 |
| SLC1A2, IL1R2, OLAH, RETN | | | | | | | | | |
| IFI44L, SIGLEC1, ISG15, HERC6, | 0.852 | 0.842 | 0.861 | 0.913 | 0.858 | 0.968 | 0.965 | 0.929 | 1 |
| SLC1A2, IL1R2, OLAH, RETN, MMP8 | | | | | | | | | |
| OAS1, IFI44L, SIGLEC1, ISG15, HERC6, | 0.852 | 0.842 | 0.861 | 0.911 | 0.857 | 0.966 | 0.967 | 0.931 | 1 |
| SLC1A2, IL1R2, RETN, MMP8 | | | | | | | | | |
| RSAD2, IFIT1, IFI44L, SIGLEC1, ISG15, | 0.852 | 0.842 | 0.861 | 0.915 | 0.860 | 0.969 | 0.941 | 0.890 | 0.993 |
| HERC6, SLC1A2, OLAH, RETN | | | | | | | | | |
| RSAD2, IFIT1, SIGLEC1, ISG15, | 0.852 | 0.843 | 0.861 | 0.913 | 0.859 | 0.966 | 0.963 | 0.925 | 1 |
| HERC6, SLC1A2, IL1R2, RETN, MMP8 | | | | | | | | | |
| RSAD2, IFIT1, IFI44L, ISG15, HERC6, | 0.852 | 0.841 | 0.862 | 0.918 | 0.868 | 0.968 | 0.924 | 0.830 | 1 |
| SLC1A2, IL1R2, FAM20A, MMP8 | | | | | | | | | |
| RSAD2, OAS1, IFIT1, IFI44L, SIGLEC1, | 0.852 | 0.842 | 0.861 | 0.911 | 0.855 | 0.967 | 0.957 | 0.910 | 1 |
| HERC6, SLC1A2, IL1R2, RETN | | | | | | | | | |
| RSAD2, IFIT1, IFI44L, SIGLEC1, | 0.852 | 0.842 | 0.861 | 0.916 | 0.862 | 0.970 | 0.949 | 0.902 | 0.996 |
| HERC6, SLC1A2, IL1R2, OLAH, RETN | | | | | | | | | |
| RSAD2, OAS1, IFIT1, HERC6, SLC1A2, | 0.852 | 0.842 | 0.862 | 0.909 | 0.851 | 0.967 | 0.932 | 0.852 | 1 |
| OLAH, FAM20A, RETN, MMP8 | | | | | | | | | |
| OAS1, IFIT1, IFI44L, SIGLEC1, ISG15, | 0.852 | 0.842 | 0.862 | 0.912 | 0.856 | 0.969 | 0.957 | 0.912 | 1 |
| SLC1A2, OLAH, RETN, MMP8 | | | | | | | | | |
| OAS1, IFIT1, IFI44L, ISG15, HERC6, | 0.852 | 0.841 | 0.862 | 0.915 | 0.864 | 0.967 | 0.924 | 0.833 | 1 |
| SLC1A2, IL1R2, FAM20A, RETN | | | | | | | | | |
| RSAD2, IFIT1, IFI44L, HERC6, SLC1A2, | 0.852 | 0.842 | 0.861 | 0.916 | 0.863 | 0.970 | 0.927 | 0.839 | 1 |
| IL1R2, FAM20A, RETN, MMP8 | | | | | | | | | |
| RSAD2, OAS1, IFIT1, IFI44L, IL1R2, | 0.852 | 0.841 | 0.862 | 0.910 | 0.849 | 0.971 | 0.945 | 0.877 | 1 |
| OLAH, FAM20A, RETN, MMP8 | | | | | | | | | |
| RSAD2, OAS1, IFIT1, IFI44L, SLC1A2, | 0.851 | 0.841 | 0.862 | 0.914 | 0.858 | 0.971 | 0.924 | 0.848 | 1.000 |
| OLAH, FAM20A, RETN, MMP8 | | | | | | | | | |
| RSAD2, IFI27, OAS1, IFIT1, ISG15, | 0.851 | 0.841 | 0.862 | 0.915 | 0.860 | 0.969 | 0.923 | 0.837 | 1 |
| HERC6, SLC1A2, FAM20A, MMP8 | | | | | | | | | |
| RSAD2, IFIT1, IFI44L, ISG15, SLC1A2, | 0.851 | 0.841 | 0.861 | 0.912 | 0.855 | 0.970 | 0.925 | 0.851 | 0.999 |
| OLAH, FAM20A, RETN, MMP8 | | | | | | | | | |
| RSAD2, OAS1, IFIT1, IFI44L, SIGLEC1, | 0.851 | 0.841 | 0.861 | 0.912 | 0.857 | 0.968 | 0.949 | 0.900 | 0.997 |
| ISG15, SLC1A2, OLAH, MMP8 | | | | | | | | | |
| RSAD2, IFIT1, ISG15, HERC6, SLC1A2, | 0.851 | 0.841 | 0.862 | 0.911 | 0.856 | 0.967 | 0.928 | 0.844 | 1 |
| IL1R2, OLAH, FAM20A, RETN | | | | | | | | | |
| RSAD2, IFIT1, IFI44L, SIGLEC1, ISG15, | 0.851 | 0.842 | 0.861 | 0.913 | 0.859 | 0.967 | 0.953 | 0.909 | 0.997 |
| HERC6, SLC1A2, IL1R2, OLAH | | | | | | | | | |
| RSAD2, IFIT1, IFI44L, SIGLEC1, ISG15, | 0.851 | 0.842 | 0.861 | 0.904 | 0.845 | 0.964 | 0.962 | 0.922 | 1 |
| HERC6, IL1R2, OLAH, MMP8 | | | | | | | | | |
| RSAD2, OAS1, IFI44L, SIGLEC1, ISG15, | 0.851 | 0.841 | 0.861 | 0.912 | 0.858 | 0.966 | 0.962 | 0.922 | 1 |
| HERC6, SLC1A2, IL1R2, OLAH | | | | | | | | | |
| OAS1, IFI44L, SIGLEC1, ISG15, HERC6, | 0.851 | 0.841 | 0.862 | 0.914 | 0.860 | 0.968 | 0.957 | 0.912 | 1 |
| SLC1A2, IL1R2, OLAH, RETN | | | | | | | | | |
| RSAD2, OAS1, IFIT1, SIGLEC1, ISG15, | 0.851 | 0.841 | 0.861 | 0.906 | 0.849 | 0.963 | 0.961 | 0.921 | 1 |
| HERC6, IL1R2, OLAH, MMP8 | | | | | | | | | |
| RSAD2, OAS1, IFI44L, SIGLEC1, | 0.851 | 0.841 | 0.862 | 0.911 | 0.853 | 0.968 | 0.963 | 0.922 | 1 |
| HERC6, SLC1A2, OLAH, RETN, MMP8 | | | | | | | | | |
| RSAD2, IFI44L, SIGLEC1, HERC6, | 0.851 | 0.841 | 0.861 | 0.915 | 0.861 | 0.969 | 0.962 | 0.923 | 1 |
| SLC1A2, IL1R2, OLAH, RETN, MMP8 | | | | | | | | | |
| RSAD2, IFIT1, IFI44L, SIGLEC1, ISG15, | 0.851 | 0.842 | 0.861 | 0.905 | 0.844 | 0.967 | 0.950 | 0.904 | 0.996 |
| HERC6, IL1R2, OLAH, RETN | | | | | | | | | |
| OAS1, IFI44L, SIGLEC1, ISG15, HERC6, | 0.851 | 0.841 | 0.861 | 0.912 | 0.859 | 0.966 | 0.961 | 0.922 | 1 |
| SLC1A2, IL1R2, OLAH, MMP8 | | | | | | | | | |
| RSAD2, IFI44L, SIGLEC1, ISG15, | 0.851 | 0.841 | 0.861 | 0.903 | 0.839 | 0.966 | 0.963 | 0.924 | 1 |
| HERC6, IL1R2, OLAH, RETN, MMP8 | | | | | | | | | |
| RSAD2, OAS1, IFI44L, SIGLEC1, ISG15, | 0.851 | 0.841 | 0.861 | 0.904 | 0.846 | 0.963 | 0.962 | 0.922 | 1 |
| HERC6, IL1R2, OLAH, MMP8 | | | | | | | | | |
| RSAD2, OAS1, IFI44L, SIGLEC1, ISG15, | 0.851 | 0.841 | 0.861 | 0.902 | 0.840 | 0.965 | 0.957 | 0.912 | 1 |
| HERC6, IL1R2, OLAH, RETN | | | | | | | | | |
| RSAD2, OAS1, IFI44L, HERC6, | 0.851 | 0.841 | 0.861 | 0.918 | 0.865 | 0.970 | 0.926 | 0.834 | 1 |
| SLC1A2, IL1R2, FAM20A, RETN, MMP8 | | | | | | | | | |
| RSAD2, OAS1, IFI44L, ISG15, SLC1A2, | 0.851 | 0.840 | 0.862 | 0.915 | 0.858 | 0.972 | 0.924 | 0.848 | 0.999 |
| OLAH, FAM20A, RETN, MMP8 | | | | | | | | | |
| RSAD2, IFI44L, SIGLEC1, ISG15, | 0.851 | 0.842 | 0.860 | 0.913 | 0.859 | 0.966 | 0.965 | 0.929 | 1 |
| HERC6, SLC1A2, IL1R2, OLAH, MMP8 | | | | | | | | | |
| RSAD2, IFIT1, ISG15, HERC6, SLC1A2, | 0.851 | 0.841 | 0.861 | 0.911 | 0.853 | 0.968 | 0.934 | 0.855 | 1 |
| OLAH, FAM20A, RETN, MMP8 | | | | | | | | | |
| RSAD2, OAS1, IFIT1, ISG15, HERC6, | 0.851 | 0.840 | 0.861 | 0.910 | 0.853 | 0.966 | 0.929 | 0.849 | 1 |
| SLC1A2, OLAH, FAM20A, MMP8 | | | | | | | | | |
| OAS1, IFIT1, IFI44L, SIGLEC1, | 0.851 | 0.841 | 0.861 | 0.914 | 0.860 | 0.968 | 0.967 | 0.932 | 1 |
| SLC1A2, IL1R2, OLAH, RETN, MMP8 | | | | | | | | | |
| RSAD2, OAS1, IFIT1, ISG15, HERC6, | 0.851 | 0.840 | 0.861 | 0.911 | 0.856 | 0.966 | 0.928 | 0.842 | 1 |
| SLC1A2, IL1R2, FAM20A, RETN | | | | | | | | | |
| RSAD2, OAS1, IFIT1, SIGLEC1, | 0.851 | 0.841 | 0.861 | 0.913 | 0.859 | 0.967 | 0.972 | 0.940 | 1 |
| SLC1A2, IL1R2, OLAH, RETN, MMP8 | | | | | | | | | |
| IFIT1, IFI44L, SIGLEC1, ISG15, | 0.851 | 0.841 | 0.860 | 0.913 | 0.859 | 0.968 | 0.967 | 0.933 | 1 |
| SLC1A2, IL1R2, OLAH, RETN, MMP8 | | | | | | | | | |
| RSAD2, OAS1, IFIT1, HERC6, SLC1A2, | 0.851 | 0.840 | 0.861 | 0.912 | 0.857 | 0.966 | 0.928 | 0.844 | 1 |
| IL1R2, OLAH, FAM20A, RETN | | | | | | | | | |
| RSAD2, IFIT1, IFI44L, SIGLEC1, | 0.851 | 0.842 | 0.860 | 0.912 | 0.857 | 0.966 | 0.964 | 0.927 | 1 |
| HERC6, SLC1A2, IL1R2, RETN, MMP8 | | | | | | | | | |
| RSAD2, OAS1, IFIT1, IFI44L, ISG15, | 0.850 | 0.840 | 0.861 | 0.914 | 0.859 | 0.969 | 0.927 | 0.847 | 1 |
| SLC1A2, IL1R2, OLAH, FAM20A | | | | | | | | | |
| RSAD2, OAS1, IFI44L, ISG15, IL1R2, | 0.850 | 0.840 | 0.861 | 0.910 | 0.848 | 0.971 | 0.945 | 0.876 | 1 |
| OLAH, FAM20A, RETN, MMP8 | | | | | | | | | |
| RSAD2, OAS1, IFI44L, ISG15, HERC6, | 0.850 | 0.839 | 0.862 | 0.914 | 0.861 | 0.967 | 0.925 | 0.835 | 1 |
| SLC1A2, IL1R2, FAM20A, RETN | | | | | | | | | |
| RSAD2, IFIT1, IFI44L, SIGLEC1, ISG15, | 0.850 | 0.840 | 0.860 | 0.905 | 0.841 | 0.968 | 0.957 | 0.915 | 0.998 |
| HERC6, OLAH, RETN, MMP8 | | | | | | | | | |
| RSAD2, OAS1, IFIT1, IFI44L, SIGLEC1, | 0.850 | 0.841 | 0.860 | 0.912 | 0.858 | 0.966 | 0.968 | 0.934 | 1 |
| SLC1A2, IL1R2, OLAH, MMP8 | | | | | | | | | |
| RSAD2, IFIT1, SIGLEC1, ISG15, | 0.850 | 0.841 | 0.860 | 0.913 | 0.858 | 0.967 | 0.968 | 0.935 | 1 |
| SLC1A2, IL1R2, OLAH, RETN, MMP8 | | | | | | | | | |
| OAS1, IFIT1, IFI44L, SIGLEC1, ISG15, | 0.850 | 0.841 | 0.860 | 0.913 | 0.859 | 0.966 | 0.967 | 0.933 | 1 |
| SLC1A2, IL1R2, OLAH, MMP8 | | | | | | | | | |
| RSAD2, OAS1, IFI44L, SIGLEC1, ISG15, | 0.850 | 0.840 | 0.860 | 0.911 | 0.856 | 0.965 | 0.958 | 0.911 | 1 |
| HERC6, SLC1A2, IL1R2, RETN | | | | | | | | | |
| RSAD2, OAS1, SIGLEC1, ISG15, | 0.850 | 0.840 | 0.860 | 0.912 | 0.858 | 0.967 | 0.971 | 0.938 | 1 |
| SLC1A2, IL1R2, OLAH, RETN, MMP8 | | | | | | | | | |
| RSAD2, OAS1, IFI44L, SIGLEC1, | 0.850 | 0.840 | 0.861 | 0.912 | 0.857 | 0.967 | 0.959 | 0.914 | 1 |
| HERC6, SLC1A2, IL1R2, OLAH, RETN | | | | | | | | | |
| RSAD2, OAS1, IFIT1, IFI44L, ISG15, | 0.850 | 0.840 | 0.861 | 0.908 | 0.847 | 0.969 | 0.927 | 0.849 | 1 |
| HERC6, FAM20A, RETN, MMP8 | | | | | | | | | |
| RSAD2, OAS1, IFIT1, SIGLEC1, ISG15, | 0.850 | 0.841 | 0.860 | 0.912 | 0.858 | 0.966 | 0.968 | 0.935 | 1 |
| SLC1A2, IL1R2, OLAH, MMP8 | | | | | | | | | |
| RSAD2, OAS1, IFI44L, SIGLEC1, | 0.850 | 0.841 | 0.860 | 0.911 | 0.855 | 0.966 | 0.968 | 0.933 | 1 |
| HERC6, SLC1A2, IL1R2, RETN, MMP8 | | | | | | | | | |
| RSAD2, IFIT1, IFI44L, ISG15, HERC6, | 0.850 | 0.840 | 0.861 | 0.915 | 0.863 | 0.967 | 0.923 | 0.831 | 1 |
| SLC1A2, IL1R2, FAM20A, RETN | | | | | | | | | |
| OAS1, IFIT1, IFI44L, SIGLEC1, ISG15, | 0.850 | 0.840 | 0.860 | 0.915 | 0.860 | 0.970 | 0.955 | 0.912 | 0.999 |
| SLC1A2, IL1R2, OLAH, RETN | | | | | | | | | |
| RSAD2, OAS1, IFIT1, IFI44L, SIGLEC1, | 0.850 | 0.840 | 0.860 | 0.912 | 0.857 | 0.968 | 0.966 | 0.928 | 1 |
| SLC1A2, IL1R2, OLAH, RETN | | | | | | | | | |
| RSAD2, OAS1, IFI44L, SIGLEC1, ISG15, | 0.850 | 0.840 | 0.860 | 0.912 | 0.856 | 0.969 | 0.953 | 0.904 | 1 |
| HERC6, SLC1A2, OLAH, RETN | | | | | | | | | |
| RSAD2, OAS1, IFIT1, IFI44L, SIGLEC1, | 0.850 | 0.840 | 0.860 | 0.911 | 0.856 | 0.967 | 0.967 | 0.931 | 1 |
| ISG15, SLC1A2, IL1R2, OLAH | | | | | | | | | |
| RSAD2, IFIT1, IFI44L, SIGLEC1, | 0.850 | 0.841 | 0.860 | 0.914 | 0.860 | 0.968 | 0.970 | 0.937 | 1 |
| SLC1A2, IL1R2, OLAH, RETN, MMP8 | | | | | | | | | |
| RSAD2, OAS1, IFIT1, ISG15, HERC6, | 0.850 | 0.840 | 0.861 | 0.912 | 0.857 | 0.968 | 0.932 | 0.851 | 1 |
| SLC1A2, OLAH, FAM20A, RETN | | | | | | | | | |
| RSAD2, OAS1, ISG15, SLC1A2, IL1R2, | 0.850 | 0.840 | 0.860 | 0.914 | 0.861 | 0.967 | 0.934 | 0.854 | 1 |
| OLAH, FAM20A, RETN, MMP8 | | | | | | | | | |
| RSAD2, IFIT1, ISG15, SLC1A2, IL1R2, | 0.850 | 0.840 | 0.860 | 0.913 | 0.859 | 0.967 | 0.938 | 0.862 | 1 |
| OLAH, FAM20A, RETN, MMP8 | | | | | | | | | |

Performance obtained for the identification of the viral or bacterial nature of the infection on the basis of the target 10-gene combinations:

TABLE 14

| | AUC | | | AUC | CI 95% | | AUC | CI 95% | |
|---|---|---|---|---|---|---|---|---|---|
| Model | model | CI 95% | | Train | Train | | Test | Test | |
| RSAD2, IFI27, OAS1, IFIT1, SIGLEC1, | 0.888 | 0.878 | 0.897 | 0.938 | 0.890 | 0.987 | 0.951 | 0.890 | 1 |
| HERC6, IL1R2, OLAH, FAM20A, MMP8 | | | | | | | | | |
| IFI27, OAS1, IFIT1, IFI44L, SIGLEC1, | 0.888 | 0.878 | 0.897 | 0.938 | 0.889 | 0.986 | 0.952 | 0.891 | 1 |
| HERC6, IL1R2, OLAH, FAM20A, MMP8 | | | | | | | | | |
| IFI27, IFIT1, IFI44L, SIGLEC1, ISG15, | 0.887 | 0.878 | 0.896 | 0.938 | 0.889 | 0.986 | 0.946 | 0.879 | 1 |
| HERC6, IL1R2, OLAH, FAM20A, MMP8 | | | | | | | | | |
| IFI27, OAS1, IFIT1, SIGLEC1, HERC6, | 0.887 | 0.877 | 0.897 | 0.938 | 0.886 | 0.989 | 0.952 | 0.891 | 1 |
| IL1R2, OLAH, FAM20A, RETN, MMP8 | | | | | | | | | |
| RSAD2, IFI27, IFIT1, IFI44L, SIGLEC1, | 0.887 | 0.878 | 0.895 | 0.938 | 0.890 | 0.987 | 0.951 | 0.889 | 1 |
| HERC6, IL1R2, OLAH, FAM20A, MMP8 | | | | | | | | | |
| IFI27, OAS1, IFIT1, SIGLEC1, ISG15, | 0.887 | 0.877 | 0.897 | 0.938 | 0.889 | 0.987 | 0.953 | 0.891 | 1 |
| HERC6, IL1R2, OLAH, FAM20A, MMP8 | | | | | | | | | |
| RSAD2, IFI27, IFIT1, SIGLEC1, ISG15, | 0.886 | 0.877 | 0.896 | 0.938 | 0.890 | 0.987 | 0.951 | 0.889 | 1 |
| HERC6, IL1R2, OLAH, FAM20A, MMP8 | | | | | | | | | |
| IFI27, OAS1, IFI44L, SIGLEC1, ISG15, | 0.886 | 0.876 | 0.895 | 0.937 | 0.888 | 0.985 | 0.948 | 0.885 | 1 |
| HERC6, IL1R2, OLAH, FAM20A, MMP8 | | | | | | | | | |
| RSAD2, IFI27, OAS1, IFIT1, IFI44L, | 0.885 | 0.876 | 0.895 | 0.934 | 0.883 | 0.986 | 0.947 | 0.885 | 1 |
| SIGLEC1, HERC6, OLAH, FAM20A, MMP8 | | | | | | | | | |
| RSAD2, IFI27, OAS1, SIGLEC1, ISG15, | 0.885 | 0.875 | 0.895 | 0.937 | 0.886 | 0.987 | 0.951 | 0.891 | 1 |
| HERC6, IL1R2, OLAH, FAM20A, MMP8 | | | | | | | | | |
| RSAD2, IFI27, OAS1, IFI44L, SIGLEC1, | 0.885 | 0.876 | 0.895 | 0.936 | 0.887 | 0.985 | 0.951 | 0.890 | 1 |
| HERC6, IL1R2, OLAH, FAM20A, MMP8 | | | | | | | | | |
| IFI27, OAS1, SIGLEC1, ISG15, HERC6, | 0.885 | 0.875 | 0.895 | 0.934 | 0.881 | 0.987 | 0.949 | 0.885 | 1 |
| IL1R2, OLAH, FAM20A, RETN, MMP8 | | | | | | | | | |
| IFI27, OAS1, IFIT1, IFI44L, SIGLEC1, | 0.885 | 0.875 | 0.895 | 0.936 | 0.887 | 0.986 | 0.951 | 0.890 | 1 |
| ISG15, IL1R2, OLAH, FAM20A, MMP8 | | | | | | | | | |
| IFI27, OAS1, IFIT1, SIGLEC1, ISG15, | 0.885 | 0.875 | 0.895 | 0.936 | 0.883 | 0.990 | 0.949 | 0.887 | 1 |
| HERC6, OLAH, FAM20A, RETN, MMP8 | | | | | | | | | |
| IFI27, OAS1, IFIT1, IFI44L, SIGLEC1, | 0.885 | 0.875 | 0.894 | 0.938 | 0.889 | 0.988 | 0.950 | 0.880 | 1 |
| HERC6, IL1R2, FAM20A, RETN, MMP8 | | | | | | | | | |
| RSAD2, IFI27, IFI44L, SIGLEC1, ISG15, | 0.885 | 0.875 | 0.894 | 0.938 | 0.889 | 0.986 | 0.948 | 0.885 | 1 |
| HERC6, IL1R2, OLAH, FAM20A, MMP8 | | | | | | | | | |
| RSAD2, IFI27, OAS1, SIGLEC1, HERC6, | 0.885 | 0.874 | 0.895 | 0.937 | 0.884 | 0.989 | 0.949 | 0.886 | 1 |
| IL1R2, OLAH, FAM20A, RETN, MMP8 | | | | | | | | | |
| RSAD2, IFI27, OAS1, IFIT1, SIGLEC1, | 0.885 | 0.875 | 0.894 | 0.937 | 0.887 | 0.987 | 0.952 | 0.891 | 1 |
| ISG15, IL1R2, OLAH, FAM20A, MMP8 | | | | | | | | | |
| RSAD2, IFI27, OAS1, IFIT1, SIGLEC1, | 0.885 | 0.874 | 0.895 | 0.937 | 0.885 | 0.989 | 0.949 | 0.887 | 1 |
| HERC6, OLAH, FAM20A, RETN, MMP8 | | | | | | | | | |
| RSAD2, IFI27, OAS1, IFIT1, SIGLEC1, | 0.885 | 0.875 | 0.894 | 0.934 | 0.881 | 0.986 | 0.945 | 0.883 | 1 |
| ISG15, HERC6, OLAH, FAM20A, MMP8 | | | | | | | | | |
| RSAD2, IFI27, OAS1, IFIT1, IFI44L, | 0.884 | 0.875 | 0.894 | 0.935 | 0.884 | 0.985 | 0.954 | 0.896 | 1 |
| SIGLEC1, ILIR2, OLAH, FAM20A, MMP8 | | | | | | | | | |
| IFI27, OAS1, IFIT1, IFI44L, SIGLEC1, | 0.884 | 0.874 | 0.895 | 0.938 | 0.886 | 0.990 | 0.947 | 0.884 | 1 |
| HERC6, OLAH, FAM20A, RETN, MMP8 | | | | | | | | | |
| IFI27, OAS1, IFIT1, SIGLEC1, ISG15, | 0.884 | 0.875 | 0.894 | 0.937 | 0.887 | 0.988 | 0.952 | 0.885 | 1 |
| HERC6, IL1R2, FAM20A, RETN, MMP8 | | | | | | | | | |
| RSAD2, IFI27, OAS1, IFIT1, IFI44L, | 0.884 | 0.875 | 0.893 | 0.934 | 0.884 | 0.983 | 0.948 | 0.881 | 1 |
| SIGLEC1, HERC6, ILIR2, FAM20A, MMP8 | | | | | | | | | |
| IFI27, IFIT1, IFI44L, SIGLEC1, HERC6, | 0.884 | 0.874 | 0.894 | 0.937 | 0.884 | 0.989 | 0.952 | 0.888 | 1 |
| IL1R2, OLAH, FAM20A, RETN, MMP8 | | | | | | | | | |
| RSAD2, IFI27, OAS1, SIGLEC1, ISG15, | 0.884 | 0.874 | 0.894 | 0.935 | 0.880 | 0.989 | 0.948 | 0.888 | 1 |
| HERC6, OLAH, FAM20A, RETN, MMP8 | | | | | | | | | |
| RSAD2, IFI27, SIGLEC1, ISG15, HERC6, | 0.884 | 0.873 | 0.894 | 0.936 | 0.884 | 0.988 | 0.950 | 0.887 | 1 |
| IL1R2, OLAH, FAM20A, RETN, MMP8 | | | | | | | | | |
| IFI27, OAS1, IFIT1, IFI44L, SIGLEC1, | 0.883 | 0.873 | 0.894 | 0.933 | 0.879 | 0.986 | 0.946 | 0.884 | 1 |
| ISG15, HERC6, OLAH, FAM20A, MMP8 | | | | | | | | | |
| RSAD2, IFI27, OAS1, IFIT1, SIGLEC1, | 0.883 | 0.874 | 0.893 | 0.937 | 0.887 | 0.988 | 0.951 | 0.883 | 1 |
| HERC6, IL1R2, FAM20A, RETN, MMP8 | | | | | | | | | |
| RSAD2, IFI27, IFIT1, SIGLEC1, HERC6, | 0.883 | 0.873 | 0.893 | 0.938 | 0.887 | 0.989 | 0.951 | 0.888 | 1 |
| IL1R2, OLAH, FAM20A, RETN, MMP8 | | | | | | | | | |
| RSAD2, IFI27, OAS1, IFIT1, IFI44L, | 0.883 | 0.875 | 0.892 | 0.934 | 0.884 | 0.984 | 0.943 | 0.880 | 1 |
| SIGLEC1, HERC6, IL1R2, OLAH, FAM20A | | | | | | | | | |
| IFI27, IFIT1, SIGLEC1, ISG15, HERC6, | 0.883 | 0.873 | 0.894 | 0.937 | 0.885 | 0.988 | 0.954 | 0.891 | 1 |
| IL1R2, OLAH, FAM20A, RETN, MMP8 | | | | | | | | | |
| IFI27, IFI44L, SIGLEC1, ISG15, HERC6, | 0.883 | 0.873 | 0.893 | 0.935 | 0.884 | 0.987 | 0.948 | 0.882 | 1 |
| IL1R2, OLAH, FAM20A, RETN, MMP8 | | | | | | | | | |
| RSAD2, IFI27, OAS1, IFI44L, SIGLEC1, | 0.883 | 0.873 | 0.893 | 0.936 | 0.886 | 0.985 | 0.952 | 0.893 | 1 |
| ISG15, IL1R2, OLAH, FAM20A, MMP8 | | | | | | | | | |
| RSAD2, IFI27, IFIT1, IFI44L, SIGLEC1, | 0.883 | 0.873 | 0.892 | 0.936 | 0.887 | 0.986 | 0.951 | 0.890 | 1 |
| ISG15, IL1R2, OLAH, FAM20A, MMP8 | | | | | | | | | |
| IFI27, IFIT1, IFI44L, SIGLEC1, ISG15, | 0.883 | 0.873 | 0.892 | 0.936 | 0.886 | 0.987 | 0.952 | 0.883 | 1 |
| HERC6, IL1R2, FAM20A, RETN, MMP8 | | | | | | | | | |
| IFI27, IFIT1, IFI44L, SIGLEC1, ISG15, | 0.883 | 0.872 | 0.893 | 0.935 | 0.881 | 0.988 | 0.952 | 0.892 | 1 |
| HERC6, OLAH, FAM20A, RETN, MMP8 | | | | | | | | | |
| IFI27, OAS1, IFI44L, SIGLEC1, HERC6, | 0.882 | 0.872 | 0.893 | 0.935 | 0.883 | 0.988 | 0.949 | 0.883 | 1 |
| ILIR2, OLAH, FAM20A, RETN, MMP8 | | | | | | | | | |
| IFI27, OAS1, IFIT1, IFI44L, SIGLEC1, | 0.882 | 0.873 | 0.891 | 0.933 | 0.881 | 0.985 | 0.940 | 0.876 | 1 |
| HERC6, IL1R2, OLAH, FAM20A, RETN | | | | | | | | | |
| RSAD2, IFI27, IFIT1, SIGLEC1, ISG15, | 0.882 | 0.872 | 0.893 | 0.936 | 0.883 | 0.990 | 0.949 | 0.887 | 1 |
| HERC6, OLAH, FAM20A, RETN, MMP8 | | | | | | | | | |
| IFI27, IFIT1, IFI44L, SIGLEC1, ISG15, | 0.882 | 0.872 | 0.892 | 0.937 | 0.886 | 0.989 | 0.952 | 0.889 | 1 |
| IL1R2, OLAH, FAM20A, RETN, MMP8 | | | | | | | | | |
| IFI27, OAS1, IFIT1, SIGLEC1, ISG15, | 0.882 | 0.872 | 0.893 | 0.933 | 0.880 | 0.987 | 0.955 | 0.896 | 1 |
| IL1R2, OLAH, FAM20A, RETN, MMP8 | | | | | | | | | |
| IFI27, IFIT1, IFI44L, SIGLEC1, ISG15, | 0.882 | 0.873 | 0.891 | 0.932 | 0.879 | 0.985 | 0.945 | 0.881 | 1 |
| HERC6, IL1R2, OLAH, FAM20A, RETN | | | | | | | | | |
| RSAD2, IFI27, OAS1, IFIT1, SIGLEC1, | 0.882 | 0.873 | 0.891 | 0.933 | 0.881 | 0.984 | 0.939 | 0.874 | 1 |
| ISG15, HERC6, IL1R2, OLAH, FAM20A | | | | | | | | | |
| IFI27, OAS1, IFIT1, IFI44L, SIGLEC1, | 0.882 | 0.873 | 0.891 | 0.932 | 0.878 | 0.985 | 0.939 | 0.876 | 1 |
| ISG15, HERC6, OLAH, FAM20A, RETN | | | | | | | | | |
| RSAD2, IFI27, IFIT1, IFI44L, SIGLEC1, | 0.882 | 0.871 | 0.892 | 0.935 | 0.883 | 0.987 | 0.947 | 0.886 | 1 |
| ISG15, HERC6, OLAH, FAM20A, MMP8 | | | | | | | | | |
| IFI27, OAS1, IFIT1, SIGLEC1, ISG15, | 0.882 | 0.872 | 0.891 | 0.932 | 0.879 | 0.985 | 0.937 | 0.872 | 1 |
| HERC6, IL1R2, OLAH, FAM20A, RETN | | | | | | | | | |
| RSAD2, IFI27, IFIT1, SIGLEC1, ISG15, | 0.882 | 0.872 | 0.891 | 0.938 | 0.887 | 0.988 | 0.950 | 0.881 | 1 |
| HERC6, IL1R2, FAM20A, RETN, MMP8 | | | | | | | | | |
| IFI27, OAS1, IFIT1, IFI44L, SIGLEC1, | 0.882 | 0.872 | 0.891 | 0.934 | 0.882 | 0.985 | 0.940 | 0.875 | 1 |
| ISG15, HERC6, IL1R2, OLAH, FAM20A | | | | | | | | | |
| IFI27, OAS1, IFIT1, IFI44L, SIGLEC1, | 0.881 | 0.872 | 0.891 | 0.934 | 0.885 | 0.983 | 0.950 | 0.882 | 1 |
| ISG15, HERC6, IL1R2, FAM20A, MMP8 | | | | | | | | | |
| RSAD2, IFI27, OAS1, IFIT1, IFI44L, | 0.881 | 0.872 | 0.891 | 0.931 | 0.877 | 0.984 | 0.938 | 0.873 | 1 |
| SIGLEC1, HERC6, OLAH, FAM20A, RETN | | | | | | | | | |
| RSAD2, IFI27, OAS1, IFIT1, SIGLEC1, | 0.881 | 0.872 | 0.891 | 0.934 | 0.885 | 0.983 | 0.952 | 0.885 | 1 |
| ISG15, HERC6, IL1R2, FAM20A, MMP8 | | | | | | | | | |
| RSAD2, IFI27, OAS1, IFIT1, SIGLEC1, | 0.881 | 0.871 | 0.891 | 0.934 | 0.880 | 0.987 | 0.954 | 0.895 | 1 |
| IL1R2, OLAH, FAM20A, RETN, MMP8 | | | | | | | | | |
| RSAD2, IFI27, IFIT1, IFI44L, SIGLEC1, | 0.881 | 0.871 | 0.891 | 0.938 | 0.888 | 0.988 | 0.950 | 0.880 | 1 |
| HERC6, IL1R2, FAM20A, RETN, MMP8 | | | | | | | | | |
| RSAD2, IFI27, OAS1, SIGLEC1, ISG15, | 0.881 | 0.871 | 0.892 | 0.934 | 0.881 | 0.987 | 0.951 | 0.889 | 1 |
| ILIR2, OLAH, FAM20A, RETN, MMP8 | | | | | | | | | |
| RSAD2, IFI27, OAS1, IFIT1, SIGLEC1, | 0.881 | 0.872 | 0.890 | 0.932 | 0.879 | 0.985 | 0.937 | 0.872 | 1 |
| ISG15, HERC6, OLAH, FAM20A, RETN | | | | | | | | | |
| RSAD2, IFI27, OAS1, IFIT1, IFI44L, | 0.881 | 0.871 | 0.891 | 0.932 | 0.879 | 0.985 | 0.939 | 0.875 | 1 |
| SIGLEC1, ISG15, HERC6, OLAH, FAM20A | | | | | | | | | |
| RSAD2, IFI27, OAS1, IFIT1, SIGLEC1, | 0.881 | 0.872 | 0.890 | 0.931 | 0.877 | 0.984 | 0.939 | 0.874 | 1 |
| HERC6, IL1R2, OLAH, FAM20A, RETN | | | | | | | | | |
| RSAD2, IFI27, OAS1, SIGLEC1, ISG15, | 0.881 | 0.871 | 0.891 | 0.936 | 0.884 | 0.987 | 0.949 | 0.880 | 1 |
| HERC6, IL1R2, FAM20A, RETN, MMP8 | | | | | | | | | |
| IFI27, OAS1, SIGLEC1, ISG15, HERC6, | 0.881 | 0.871 | 0.891 | 0.939 | 0.895 | 0.984 | 0.947 | 0.879 | 1 |
| SLC1A2, IL1R2, OLAH, FAM20A, MMP8 | | | | | | | | | |
| IFI27, IFIT1, IFI44L, SIGLEC1, ISG15, | 0.881 | 0.872 | 0.890 | 0.943 | 0.90 | 0.986 | 0.946 | 0.878 | 1 |
| SLC1A2, IL1R2, OLAH, FAM20A, MMP8 | | | | | | | | | |
| RSAD2, IFI27, IFIT1, IFI44L, SIGLEC1, | 0.881 | 0.872 | 0.890 | 0.933 | 0.881 | 0.985 | 0.943 | 0.879 | 1 |
| ISG15, HERC6, IL1R2, OLAH, FAM20A | | | | | | | | | |
| IFI27, OAS1, IFIT1, SIGLEC1, HERC6, | 0.881 | 0.871 | 0.891 | 0.941 | 0.897 | 0.985 | 0.946 | 0.880 | 1 |
| SLC1A2, IL1R2, OLAH, FAM20A, MMP8 | | | | | | | | | |
| IFI27, IFI44L, SIGLEC1, ISG15, HERC6, | 0.881 | 0.871 | 0.890 | 0.941 | 0.897 | 0.985 | 0.949 | 0.882 | 1 |
| SLC1A2, IL1R2, OLAH, FAM20A, MMP8 | | | | | | | | | |
| RSAD2, IFI27, IFI44L, SIGLEC1, HERC6, | 0.881 | 0.870 | 0.891 | 0.936 | 0.884 | 0.988 | 0.952 | 0.890 | 1 |
| IL1R2, OLAH, FAM20A, RETN, MMP8 | | | | | | | | | |
| IFI27, OAS1, IFIT1, IFI44L, SIGLEC1, | 0.881 | 0.870 | 0.891 | 0.934 | 0.880 | 0.987 | 0.957 | 0.901 | 1 |
| IL1R2, OLAH, FAM20A, RETN, MMP8 | | | | | | | | | |
| IFI27, OAS1, IFI44L, SIGLEC1, ISG15, | 0.881 | 0.870 | 0.891 | 0.934 | 0.881 | 0.988 | 0.947 | 0.881 | 1 |
| HERC6, OLAH, FAM20A, RETN, MMP8 | | | | | | | | | |
| RSAD2, IFI27, IFIT1, IFI44L, SIGLEC1, | 0.881 | 0.870 | 0.892 | 0.937 | 0.884 | 0.990 | 0.949 | 0.887 | 1 |
| HERC6, OLAH, FAM20A, RETN, MMP8 | | | | | | | | | |
| IFI27, OAS1, IFI44L, SIGLEC1, ISG15, | 0.881 | 0.870 | 0.891 | 0.935 | 0.883 | 0.988 | 0.951 | 0.889 | 1 |
| IL1R2, OLAH, FAM20A, RETN, MMP8 | | | | | | | | | |
| RSAD2, IFI27, OAS1, IFIT1, IFI44L, | 0.881 | 0.871 | 0.891 | 0.933 | 0.881 | 0.984 | 0.932 | 0.858 | 1 |
| HERC6, IL1R2, OLAH, FAM20A, MMP8 | | | | | | | | | |
| IFI27, IFIT1, SIGLEC1, ISG15, HERC6, | 0.881 | 0.871 | 0.891 | 0.942 | 0.899 | 0.986 | 0.949 | 0.883 | 1 |
| SLC1A2, IL1R2, OLAH, FAM20A, MMP8 | | | | | | | | | |
| RSAD2, IFI27, IFIT1, IFI44L, SIGLEC1, | 0.881 | 0.872 | 0.890 | 0.932 | 0.88 | 0.983 | 0.953 | 0.886 | 1 |
| ISG15, HERC6, IL1R2, FAM20A, MMP8 | | | | | | | | | |
| RSAD2, IFI27, OAS1, IFIT1, SIGLEC1, | 0.881 | 0.870 | 0.891 | 0.939 | 0.893 | 0.986 | 0.941 | 0.876 | 1 |
| HERC6, SLC1A2, OLAH, FAM20A, MMP8 | | | | | | | | | |
| IFI27, OAS1, IFIT1, SIGLEC1, HERC6, | 0.881 | 0.870 | 0.891 | 0.940 | 0.894 | 0.986 | 0.943 | 0.877 | 1 |
| SLC1A2, OLAH, FAM20A, RETN, MMP8 | | | | | | | | | |
| IFI27, OAS1, IFIT1, IFI44L, SIGLEC1, | 0.881 | 0.870 | 0.891 | 0.939 | 0.892 | 0.986 | 0.942 | 0.879 | 1 |
| HERC6, SLC1A2, OLAH, FAM20A, MMP8 | | | | | | | | | |
| IFI27, OAS1, IFIT1, IFI44L, SIGLEC1, | 0.880 | 0.870 | 0.891 | 0.932 | 0.879 | 0.986 | 0.942 | 0.873 | 1 |
| ISG15, HERC6, FAM20A, RETN, MMP8 | | | | | | | | | |
| RSAD2, IFI27, SIGLEC1, ISG15, HERC6, | 0.880 | 0.870 | 0.890 | 0.941 | 0.897 | 0.985 | 0.950 | 0.885 | 1 |
| SLC1A2, IL1R2, OLAH, FAM20A, MMP8 | | | | | | | | | |
| IFI27, OAS1, IFIT1, SIGLEC1, ISG15, | 0.880 | 0.870 | 0.890 | 0.943 | 0.899 | 0.986 | 0.947 | 0.880 | 1 |
| SLC1A2, IL1R2, OLAH, FAM20A, MMP8 | | | | | | | | | |
| RSAD2, IFI27, IFIT1, SIGLEC1, ISG15, | 0.880 | 0.871 | 0.889 | 0.933 | 0.881 | 0.986 | 0.940 | 0.875 | 1 |
| HERC6, IL1R2, OLAH, FAM20A, RETN | | | | | | | | | |
| RSAD2, IFI27, OAS1, IFI44L, SIGLEC1, | 0.880 | 0.870 | 0.891 | 0.933 | 0.879 | 0.986 | 0.946 | 0.884 | 1 |
| ISG15, HERC6, OLAH, FAM20A, MMP8 | | | | | | | | | |
| RSAD2, IFI27, IFIT1, SIGLEC1, ISG15, | 0.880 | 0.870 | 0.890 | 0.935 | 0.883 | 0.988 | 0.953 | 0.892 | 1 |
| ILIR2, OLAH, FAM20A, RETN, MMP8 | | | | | | | | | |
| IFI27, OAS1, IFI44L, SIGLEC1, ISG15, | 0.880 | 0.870 | 0.891 | 0.938 | 0.888 | 0.988 | 0.948 | 0.877 | 1 |
| HERC6, IL1R2, FAM20A, RETN, MMP8 | | | | | | | | | |
| RSAD2, IFI27, IFIT1, SIGLEC1, ISG15, | 0.880 | 0.870 | 0.890 | 0.943 | 0.900 | 0.986 | 0.950 | 0.886 | 1 |
| SLC1A2, IL1R2, OLAH, FAM20A, MMP8 | | | | | | | | | |
| RSAD2, IFI27, IFIT1, SIGLEC1, HERC6, | 0.880 | 0.870 | 0.890 | 0.940 | 0.895 | 0.985 | 0.952 | 0.891 | 1 |
| SLC1A2, IL1R2, OLAH, FAM20A, MMP8 | | | | | | | | | |
| IFI27, OAS1, IFIT1, SIGLEC1, ISG15, | 0.880 | 0.869 | 0.891 | 0.942 | 0.897 | 0.987 | 0.942 | 0.877 | 1 |
| HERC6, SLC1A2, OLAH, FAM20A, MMP8 | | | | | | | | | |
| RSAD2, IFI27, OAS1, IFIT1, SIGLEC1, | 0.880 | 0.870 | 0.890 | 0.933 | 0.879 | 0.986 | 0.942 | 0.873 | 1 |
| ISG15, HERC6, FAM20A, RETN, MMP8 | | | | | | | | | |
| RSAD2, IFI27, OAS1, IFI44L, SIGLEC1, | 0.880 | 0.869 | 0.890 | 0.936 | 0.883 | 0.989 | 0.948 | 0.886 | 1 |
| HERC6, OLAH, FAM20A, RETN, MMP8 | | | | | | | | | |
| RSAD2, IFI27, OAS1, SIGLEC1, ISG15, | 0.880 | 0.870 | 0.890 | 0.940 | 0.896 | 0.985 | 0.947 | 0.879 | 1 |
| SLC1A2, IL1R2, OLAH, FAM20A, MMP8 | | | | | | | | | |
| RSAD2, IFI27, OAS1, IFIT1, IFI44L, | 0.880 | 0.869 | 0.890 | 0.930 | 0.874 | 0.987 | 0.949 | 0.891 | 1 |
| SIGLEC1, ISG15, OLAH, FAM20A, MMP8 | | | | | | | | | |
| RSAD2, IFI27, OAS1, IFIT1, SIGLEC1, | 0.880 | 0.870 | 0.889 | 0.939 | 0.892 | 0.985 | 0.947 | 0.881 | 1 |
| SLC1A2, IL1R2, OLAH, FAM20A, MMP8 | | | | | | | | | |
| IFI27, OAS1, IFI44L, SIGLEC1, ISG15, | 0.880 | 0.870 | 0.889 | 0.932 | 0.880 | 0.984 | 0.942 | 0.876 | 1 |
| HERC6, IL1R2, OLAH, FAM20A, RETN | | | | | | | | | |
| IFI27, IFIT1, IFI44L, SIGLEC1, HERC6, | 0.880 | 0.870 | 0.889 | 0.939 | 0.894 | 0.985 | 0.952 | 0.888 | 1 |
| SLC1A2, ILIR2, OLAH, FAM20A, MMP8 | | | | | | | | | |
| RSAD2, IFI27, OAS1, IFI44L, SIGLEC1, | 0.880 | 0.870 | 0.889 | 0.932 | 0.880 | 0.984 | 0.941 | 0.875 | 1 |
| ISG15, HERC6, IL1R2, OLAH, FAM20A | | | | | | | | | |
| RSAD2, IFI27, OAS1, SIGLEC1, ISG15, | 0.880 | 0.869 | 0.890 | 0.937 | 0.888 | 0.985 | 0.940 | 0.875 | 1 |
| HERC6, SLC1A2, OLAH, FAM20A, MMP8 | | | | | | | | | |
| RSAD2, IFI27, OAS1, IFI44L, ISG15, | 0.880 | 0.869 | 0.890 | 0.931 | 0.879 | 0.983 | 0.928 | 0.854 | 1 |
| HERC6, IL1R2, OLAH, FAM20A, MMP8 | | | | | | | | | |
| IFI27, IFIT1, IFI44L, SIGLEC1, ISG15, | 0.879 | 0.869 | 0.890 | 0.939 | 0.893 | 0.986 | 0.949 | 0.886 | 1 |
| HERC6, SLC1A2, OLAH, FAM20A, MMP8 | | | | | | | | | |
| RSAD2, IFI27, OAS1, IFIT1, IFI44L, | 0.879 | 0.870 | 0.889 | 0.930 | 0.877 | 0.984 | 0.934 | 0.861 | 1 |
| SIGLEC1, ISG15, HERC6, FAM20A, RETN | | | | | | | | | |
| IFI27, OAS1, IFIT1, SIGLEC1, HERC6, | 0.879 | 0.869 | 0.890 | 0.940 | 0.896 | 0.984 | 0.947 | 0.875 | 1 |
| SLC1A2, IL1R2, FAM20A, RETN, MMP8 | | | | | | | | | |
| RSAD2, IFI27, OAS1, IFIT1, SIGLEC1, | 0.879 | 0.869 | 0.889 | 0.939 | 0.891 | 0.987 | 0.945 | 0.880 | 1 |
| ISG15, SLC1A2, OLAH, FAM20A, MMP8 | | | | | | | | | |
| RSAD2, IFI27, OAS1, IFIT1, HERC6, IL1R2, | 0.879 | 0.869 | 0.889 | 0.934 | 0.882 | 0.986 | 0.933 | 0.861 | 1 |
| OLAH, FAM20A, RETN, MMP8 | | | | | | | | | |
| RSAD2, IFI27, OAS1, IFIT1, SIGLEC1, | 0.879 | 0.869 | 0.890 | 0.931 | 0.874 | 0.988 | 0.954 | 0.900 | 1 |
| ISG15, OLAH, FAM20A, RETN, MMP8 | | | | | | | | | |
| IFI27, OAS1, IFIT1, IFI44L, HERC6, IL1R2, | 0.879 | 0.869 | 0.889 | 0.934 | 0.881 | 0.986 | 0.932 | 0.859 | 1 |
| OLAH, FAM20A, RETN, MMP8 | | | | | | | | | |
| IFI27, OAS1, SIGLEC1, ISG15, HERC6, | 0.879 | 0.868 | 0.890 | 0.940 | 0.894 | 0.987 | 0.942 | 0.876 | 1 |
| SLC1A2, OLAH, FAM20A, RETN, MMP8 | | | | | | | | | |
| IFI27, OAS1, IFIT1, IFI44L, SIGLEC1, | 0.879 | 0.869 | 0.889 | 0.935 | 0.884 | 0.986 | 0.952 | 0.885 | 1 |
| ISG15, IL1R2, FAM20A, RETN, MMP8 | | | | | | | | | |
| RSAD2, IFI27, OAS1, IFI44L, SIGLEC1, | 0.879 | 0.868 | 0.890 | 0.937 | 0.886 | 0.987 | 0.948 | 0.878 | 1 |
| HERC6, IL1R2, FAM20A, RETN, MMP8 | | | | | | | | | |
| RSAD2, IFI27, OAS1, IFIT1, IFI44L, | 0.879 | 0.869 | 0.889 | 0.937 | 0.887 | 0.988 | 0.945 | 0.882 | 1 |
| SIGLEC1, SLC1A2, OLAH, FAM20A, | | | | | | | | | |
| MMP8 | | | | | | | | | |
| IFI27, OAS1, IFIT1, IFI44L, SIGLEC1, | 0.879 | 0.868 | 0.890 | 0.939 | 0.890 | 0.987 | 0.945 | 0.880 | 1 |
| ISG15, SLC1A2, OLAH, FAM20A, MMP8 | | | | | | | | | |
| RSAD2, IFI27, OASI, SIGLEC1, HERC6, | 0.879 | 0.869 | 0.889 | 0.938 | 0.892 | 0.984 | 0.941 | 0.874 | 1 |
| SLC1A2, IL1R2, OLAH, FAM20A, MMP8 | | | | | | | | | |
| RSAD2, IFI27, OAS1, IFIT1, IFI44L, | 0.879 | 0.868 | 0.889 | 0.929 | 0.872 | 0.986 | 0.955 | 0.901 | 1 |
| SIGLEC1, OLAH, FAM20A, RETN, MMP8 | | | | | | | | | |
| RSAD2, IFI27, IFIT1, IFI44L, SIGLEC1, | 0.879 | 0.869 | 0.889 | 0.932 | 0.878 | 0.986 | 0.938 | 0.872 | 1 |
| HERC6, IL1R2, OLAH, FAM20A, RETN | | | | | | | | | |
| IFI27, OAS1, IFIT1, IFI44L, SIGLEC1, | 0.879 | 0.869 | 0.889 | 0.937 | 0.893 | 0.980 | 0.951 | 0.884 | 1 |
| HERC6, SLC1A2, IL1R2, FAM20A, MMP8 | | | | | | | | | |
| RSAD2, IFI27, IFIT1, IFI44L, SIGLEC1, | 0.879 | 0.869 | 0.889 | 0.933 | 0.879 | 0.986 | 0.940 | 0.876 | 1 |
| ISG15, HERC6, OLAH, FAM20A, RETN | | | | | | | | | |
| IFI27, OAS1, IFIT1, IFI44L, SIGLEC1, | 0.879 | 0.869 | 0.888 | 0.934 | 0.884 | 0.985 | 0.945 | 0.878 | 1 |
| ISG15, HERC6, IL1R2, FAM20A, RETN | | | | | | | | | |
| IFI27, OAS1, IFI44L, ISG15, HERC6, IL1R2, | 0.879 | 0.868 | 0.889 | 0.932 | 0.879 | 0.985 | 0.932 | 0.859 | 1 |
| OLAH, FAM20A, RETN, MMP8 | | | | | | | | | |
| RSAD2, IFI27, IFIT1, IFI44L, SIGLEC1, | 0.879 | 0.869 | 0.889 | 0.938 | 0.891 | 0.986 | 0.945 | 0.882 | 1 |
| HERC6, SLC1A2, OLAH, FAM20A, MMP8 | | | | | | | | | |
| IFI27, OAS1, SIGLEC1, ISG15, SLC1A2, | 0.879 | 0.869 | 0.889 | 0.941 | 0.897 | 0.986 | 0.946 | 0.877 | 1 |
| IL1R2, OLAH, FAM20A, RETN, MMP8 | | | | | | | | | |
| IFI27, OAS1, IFIT1, ISG15, HERC6, IL1R2, | 0.879 | 0.868 | 0.889 | 0.932 | 0.879 | 0.985 | 0.928 | 0.853 | 1 |
| OLAH, FAM20A, RETN, MMP8 | | | | | | | | | |
| IFI27, OAS1, IFIT1, IFI44L, ISG15, HERC6, | 0.878 | 0.868 | 0.889 | 0.932 | 0.880 | 0.983 | 0.930 | 0.858 | 1 |
| IL1R2, OLAH, FAM20A, MMP8 | | | | | | | | | |
| RSAD2, IFI27, OAS1, SIGLEC1, ISG15, | 0.878 | 0.868 | 0.888 | 0.931 | 0.878 | 0.984 | 0.941 | 0.875 | 1 |
| HERC6, IL1R2, OLAH, FAM20A, RETN | | | | | | | | | |
| RSAD2, IFI27, OAS1, IFIT1, IFI44L, | 0.878 | 0.869 | 0.888 | 0.936 | 0.888 | 0.984 | 0.940 | 0.876 | 1 |
| SIGLEC1, HERC6, SLC1A2, OLAH, | | | | | | | | | |
| FAM20A | | | | | | | | | |
| RSAD2, IFI27, OAS1, IFI44L, SIGLEC1, | 0.878 | 0.868 | 0.888 | 0.934 | 0.885 | 0.984 | 0.949 | 0.880 | 1 |
| ISG15, HERC6, IL1R2, FAM20A, MMP8 | | | | | | | | | |
| RSAD2, IFI27, OAS1, IFIT1, ISG15, HERC6, | 0.878 | 0.868 | 0.889 | 0.930 | 0.878 | 0.982 | 0.932 | 0.862 | 1 |
| ILIR2, OLAH, FAM20A, MMP8 | | | | | | | | | |
| IFI27, OAS1, SIGLEC1, HERC6, SLC1A2, | 0.878 | 0.868 | 0.889 | 0.938 | 0.891 | 0.984 | 0.945 | 0.876 | 1 |
| IL1R2, OLAH, FAM20A, RETN, MMP8 | | | | | | | | | |
| RSAD2, IFI27, OAS1, IFIT1, IFI44L, | 0.878 | 0.869 | 0.887 | 0.930 | 0.875 | 0.984 | 0.947 | 0.886 | 1 |
| SIGLEC1, ISG15, IL1R2, OLAH, FAM20A | | | | | | | | | |
| RSAD2, IFI27, OAS1, ISG15, HERC6, | 0.878 | 0.868 | 0.889 | 0.934 | 0.882 | 0.986 | 0.932 | 0.858 | 1 |
| ILIR2, OLAH, FAM20A, RETN, MMP8 | | | | | | | | | |
| RSAD2, IFI27, IFIT1, IFI44L, SIGLEC1, | 0.878 | 0.869 | 0.888 | 0.931 | 0.877 | 0.985 | 0.941 | 0.869 | 1 |
| ISG15, HERC6, FAM20A, RETN, MMP8 | | | | | | | | | |
| RSAD2, IFI27, OAS1, IFI44L, HERC6, | 0.878 | 0.867 | 0.889 | 0.933 | 0.881 | 0.986 | 0.929 | 0.855 | 1 |
| ILIR2, OLAH, FAM20A, RETN, MMP8 | | | | | | | | | |
| IFI27, OAS1, IFIT1, IFI44L, SIGLEC1, | 0.878 | 0.869 | 0.888 | 0.929 | 0.873 | 0.985 | 0.946 | 0.884 | 1 |
| ISG15, IL1R2, OLAH, FAM20A, RETN | | | | | | | | | |
| RSAD2, IFI27, OAS1, IFI44L, SIGLEC1, | 0.878 | 0.868 | 0.889 | 0.935 | 0.882 | 0.988 | 0.952 | 0.893 | 1 |
| IL1R2, OLAH, FAM20A, RETN, MMP8 | | | | | | | | | |
| RSAD2, IFI27, OAS1, IFIT1, IFI44L, | 0.878 | 0.868 | 0.888 | 0.935 | 0.885 | 0.984 | 0.943 | 0.876 | 1 |
| SIGLEC1, HERC6, IL1R2, FAM20A, RETN | | | | | | | | | |
| RSAD2, IFI27, OAS1, IFIT1, IFI44L, | 0.878 | 0.868 | 0.888 | 0.933 | 0.879 | 0.987 | 0.942 | 0.873 | 1 |
| SIGLEC1, HERC6, FAM20A, RETN, MMP8 | | | | | | | | | |
| IFI27, OAS1, IFIT1, IFI44L, SIGLEC1, | 0.878 | 0.868 | 0.888 | 0.939 | 0.893 | 0.985 | 0.933 | 0.863 | 1 |
| ISG15, HERC6, SLC1A2, OLAH, FAM20A | | | | | | | | | |
| RSAD2, IFI27, IFIT1, IFI44L, SIGLEC1, | 0.878 | 0.868 | 0.888 | 0.938 | 0.892 | 0.984 | 0.952 | 0.891 | 1 |
| SLC1A2, ILIR2, OLAH, FAM20A, MMP8 | | | | | | | | | |
| IFI27, IFI44L, SIGLEC1, ISG15, SLC1A2, | 0.878 | 0.868 | 0.888 | 0.941 | 0.897 | 0.985 | 0.948 | 0.879 | 1 |
| IL1R2, OLAH, FAM20A, RETN, MMP8 | | | | | | | | | |
| RSAD2, IFI27, OAS1, IFIT1, IFI44L, | 0.878 | 0.867 | 0.889 | 0.932 | 0.877 | 0.986 | 0.930 | 0.857 | 1 |
| HERC6, OLAH, FAM20A, RETN, MMP8 | | | | | | | | | |
| RSAD2, IFI27, SIGLEC1, ISG15, SLC1A2, | 0.878 | 0.868 | 0.888 | 0.944 | 0.902 | 0.987 | 0.948 | 0.881 | 1 |
| IL1R2, OLAH, FAM20A, RETN, MMP8 | | | | | | | | | |
| RSAD2, IFI27, OAS1, IFIT1, SIGLEC1, | 0.878 | 0.869 | 0.887 | 0.936 | 0.886 | 0.986 | 0.943 | 0.876 | 1 |
| ISG15, HERC6, IL1R2, FAM20A, RETN | | | | | | | | | |
| IFI27, OAS1, IFI44L, SIGLEC1, ISG15, | 0.878 | 0.868 | 0.888 | 0.941 | 0.897 | 0.984 | 0.947 | 0.879 | 1 |
| SLC1A2, IL1R2, OLAH, FAM20A, MMP8 | | | | | | | | | |
| RSAD2, IFI27, IFIT1, SIGLEC1, ISG15, | 0.878 | 0.867 | 0.889 | 0.941 | 0.895 | 0.987 | 0.941 | 0.875 | 1 |
| HERC6, SLC1A2, OLAH, FAM20A, MMP8 | | | | | | | | | |
| RSAD2, IFI27, IFIT1, IFI44L, HERC6, | 0.878 | 0.867 | 0.888 | 0.931 | 0.878 | 0.985 | 0.933 | 0.861 | 1 |
| IL1R2, OLAH, FAM20A, RETN, MMP8 | | | | | | | | | |
| RSAD2, IFI27, IFI44L, SIGLEC1, ISG15, | 0.878 | 0.867 | 0.889 | 0.936 | 0.884 | 0.988 | 0.950 | 0.887 | 1 |
| IL1R2, OLAH, FAM20A, RETN, MMP8 | | | | | | | | | |
| RSAD2, IFI27, OAS1, IFIT1, SIGLEC1, | 0.878 | 0.868 | 0.888 | 0.935 | 0.884 | 0.987 | 0.954 | 0.891 | 1 |
| ISG15, IL1R2, FAM20A, RETN, MMP8 | | | | | | | | | |
| RSAD2, IFI27, IFIT1, IFI44L, SIGLEC1, | 0.878 | 0.867 | 0.889 | 0.935 | 0.882 | 0.988 | 0.953 | 0.895 | 1 |
| IL1R2, OLAH, FAM20A, RETN, MMP8 | | | | | | | | | |
| IFI27, OAS1, IFIT1, IFI44L, SIGLEC1, | 0.878 | 0.867 | 0.888 | 0.939 | 0.893 | 0.985 | 0.946 | 0.879 | 1 |
| SLC1A2, IL1R2, OLAH, FAM20A, MMP8 | | | | | | | | | |
| RSAD2, IFI27, IFI44L, SIGLEC1, ISG15, | 0.878 | 0.868 | 0.888 | 0.938 | 0.887 | 0.988 | 0.950 | 0.880 | 1 |
| HERC6, IL1R2, FAM20A, RETN, MMP8 | | | | | | | | | |
| RSAD2, IFI27, OAS1, IFIT1, ISG15, HERC6, | 0.878 | 0.867 | 0.888 | 0.930 | 0.875 | 0.985 | 0.934 | 0.863 | 1 |
| OLAH, FAM20A, RETN, MMP8 | | | | | | | | | |
| RSAD2, IFI27, OAS1, IFIT1, IFI44L, | 0.877 | 0.868 | 0.887 | 0.933 | 0.883 | 0.983 | 0.952 | 0.885 | 1 |
| SIGLEC1, ISG15, IL1R2, FAM20A, MMP8 | | | | | | | | | |
| IFI27, OAS1, IFIT1, IFI44L, ISG15, HERC6, | 0.877 | 0.867 | 0.888 | 0.932 | 0.878 | 0.986 | 0.932 | 0.859 | 1 |
| OLAH, FAM20A, RETN, MMP8 | | | | | | | | | |
| IFI27, OAS1, IFIT1, IFI44L, SIGLEC1, | 0.877 | 0.867 | 0.888 | 0.937 | 0.891 | 0.984 | 0.937 | 0.871 | 1 |
| HERC6, SLC1A2, OLAH, FAM20A, RETN | | | | | | | | | |
| RSAD2, IFI27, IFIT1, IFI44L, SIGLEC1, | 0.877 | 0.867 | 0.888 | 0.939 | 0.891 | 0.987 | 0.943 | 0.878 | 1 |
| ISG15, SLC1A2, OLAH, FAM20A, MMP8 | | | | | | | | | |
| RSAD2, IFI27, OAS1, IFIT1, IFI44L, | 0.877 | 0.868 | 0.887 | 0.933 | 0.883 | 0.983 | 0.950 | 0.886 | 1 |
| SIGLEC1, ISG15, HERC6, IL1R2, FAM20A | | | | | | | | | |
| RSAD2, IFI27, OAS1, IFIT1, SIGLEC1, | 0.877 | 0.868 | 0.887 | 0.928 | 0.872 | 0.985 | 0.947 | 0.886 | 1 |
| ISG15, IL1R2, OLAH, FAM20A, RETN | | | | | | | | | |
| RSAD2, IFI27, IFI44L, SIGLEC1, ISG15, | 0.877 | 0.867 | 0.888 | 0.942 | 0.899 | 0.985 | 0.949 | 0.884 | 1 |
| SLC1A2, IL1R2, OLAH, FAM20A, MMP8 | | | | | | | | | |
| IFI27, IFIT1, IFI44L, SIGLEC1, HERC6, | 0.877 | 0.868 | 0.887 | 0.939 | 0.893 | 0.985 | 0.954 | 0.886 | 1 |
| SLC1A2, IL1R2, FAM20A, RETN, MMP8 | | | | | | | | | |
| RSAD2, IFI27, OAS1, SIGLEC1, HERC6, | 0.877 | 0.867 | 0.888 | 0.939 | 0.891 | 0.987 | 0.945 | 0.881 | 1 |
| SLC1A2, OLAH, FAM20A, RETN, MMP8 | | | | | | | | | |
| IFI27, OAS1, IFI44L, SIGLEC1, ISG15, | 0.877 | 0.867 | 0.888 | 0.937 | 0.889 | 0.985 | 0.943 | 0.879 | 1 |
| HERC6, SLC1A2, OLAH, FAM20A, MMP8 | | | | | | | | | |
| IFI27, OAS1, IFIT1, SIGLEC1, ISG15, | 0.877 | 0.867 | 0.888 | 0.940 | 0.895 | 0.984 | 0.930 | 0.860 | 1 |
| HERC6, SLC1A2, IL1R2, OLAH, FAM20A | | | | | | | | | |
| RSAD2, IFI27, IFI44L, SIGLEC1, HERC6, | 0.877 | 0.867 | 0.888 | 0.938 | 0.892 | 0.984 | 0.949 | 0.885 | 1 |
| SLC1A2, IL1R2, OLAH, FAM20A, MMP8 | | | | | | | | | |
| IFI27, OAS1, IFIT1, IFI44L, SIGLEC1, | 0.877 | 0.866 | 0.889 | 0.929 | 0.873 | 0.986 | 0.954 | 0.898 | 1 |
| ISG15, OLAH, FAM20A, RETN, MMP8 | | | | | | | | | |
| IFI27, IFIT1, SIGLEC1, ISG15, SLC1A2, | 0.877 | 0.867 | 0.887 | 0.944 | 0.901 | 0.987 | 0.948 | 0.879 | 1 |
| ILIR2, OLAH, FAM20A, RETN, MMP8 | | | | | | | | | |
| RSAD2, IFI27, IFIT1, SIGLEC1, HERC6, | 0.877 | 0.868 | 0.887 | 0.940 | 0.894 | 0.986 | 0.950 | 0.881 | 1 |
| SLC1A2, IL1R2, FAM20A, RETN, MMP8 | | | | | | | | | |
| RSAD2, IFI27, IFIT1, ISG15, HERC6, | 0.877 | 0.867 | 0.887 | 0.933 | 0.880 | 0.985 | 0.932 | 0.860 | 1 |
| ILIR2, OLAH, FAM20A, RETN, MMP8 | | | | | | | | | |
| RSAD2, IFI27, OAS1, IFIT1, IFI44L, | 0.877 | 0.867 | 0.888 | 0.934 | 0.882 | 0.985 | 0.954 | 0.893 | 1 |
| SIGLEC1, IL1R2, FAM20A, RETN, MMP8 | | | | | | | | | |
| RSAD2, IFI27, IFIT1, SIGLEC1, ISG15, | 0.877 | 0.867 | 0.887 | 0.938 | 0.889 | 0.986 | 0.948 | 0.885 | 1 |
| SLC1A2, OLAH, FAM20A, RETN, MMP8 | | | | | | | | | |
| RSAD2, IFI27, OAS1, IFI44L, SIGLEC1, | 0.877 | 0.867 | 0.887 | 0.932 | 0.880 | 0.984 | 0.943 | 0.878 | 1 |
| HERC6, IL1R2, OLAH, FAM20A, RETN | | | | | | | | | |
| IFI27, SIGLEC1, ISG15, HERC6, SLC1A2, | 0.877 | 0.867 | 0.887 | 0.940 | 0.895 | 0.984 | 0.947 | 0.877 | 1 |
| IL1R2, OLAH, FAM20A, RETN, MMP8 | | | | | | | | | |
| IFI27, OAS1, IFI44L, SIGLEC1, HERC6, | 0.877 | 0.866 | 0.888 | 0.936 | 0.889 | 0.983 | 0.945 | 0.878 | 1 |
| SLC1A2, IL1R2, OLAH, FAM20A, MMP8 | | | | | | | | | |
| RSAD2, IFI27, SIGLEC1, HERC6, SLC1A2, | 0.877 | 0.866 | 0.888 | 0.939 | 0.892 | 0.986 | 0.947 | 0.881 | 1 |
| IL1R2, OLAH, FAM20A, RETN, MMP8 | | | | | | | | | |
| RSAD2, IFI27, OAS1, IFIT1, SIGLEC1, | 0.877 | 0.867 | 0.887 | 0.936 | 0.890 | 0.983 | 0.940 | 0.874 | 1 |
| HERC6, SLC1A2, IL1R2, OLAH, FAM20A | | | | | | | | | |
| RSAD2, IFI27, IFIT1, IFI44L, ISG 15, | 0.877 | 0.866 | 0.888 | 0.932 | 0.881 | 0.983 | 0.932 | 0.861 | 1 |
| HERC6, IL1R2, OLAH, FAM20A, MMP8 | | | | | | | | | |
| IFI27, OAS1, IFIT1, SIGLEC1, ISG15, | 0.877 | 0.866 | 0.887 | 0.937 | 0.887 | 0.986 | 0.947 | 0.883 | 1 |
| SLC1A2, OLAH, FAM20A, RETN, MMP8 | | | | | | | | | |
| RSAD2, IFI27, IFI44L, SIGLEC1, ISG15, | 0.877 | 0.867 | 0.887 | 0.931 | 0.878 | 0.983 | 0.940 | 0.874 | 1 |
| HERC6, IL1R2, OLAH, FAM20A, RETN | | | | | | | | | |
| RSAD2, IFI27, OAS1, IFI44L, SIGLEC1, | 0.877 | 0.867 | 0.887 | 0.930 | 0.876 | 0.984 | 0.943 | 0.878 | 1 |
| ISG15, HERC6, OLAH, FAM20A, RETN | | | | | | | | | |
| RSAD2, IFI27, IFIT1, SIGLEC1, HERC6, | 0.877 | 0.866 | 0.888 | 0.940 | 0.893 | 0.987 | 0.950 | 0.889 | 1 |
| SLC1A2, OLAH, FAM20A, RETN, MMP8 | | | | | | | | | |
| RSAD2, IFI27, IFIT1, IFI44L, SIGLEC1, | 0.877 | 0.867 | 0.887 | 0.937 | 0.886 | 0.987 | 0.951 | 0.882 | 1 |
| ISG15, IL1R2, FAM20A, RETN, MMP8 | | | | | | | | | |
| IFI27, IFIT1, IFI44L, SIGLEC1, ISG15, | 0.877 | 0.867 | 0.886 | 0.938 | 0.895 | 0.981 | 0.953 | 0.885 | 1 |
| HERC6, SLC1A2, ILIR2, FAM20A, MMP8 | | | | | | | | | |
| IFI27, IFIT1, IFI44L, SIGLEC1, ISG15, | 0.877 | 0.867 | 0.886 | 0.940 | 0.895 | 0.984 | 0.939 | 0.873 | 1 |
| HERC6, SLC1A2, IL1R2, OLAH, FAM20A | | | | | | | | | |
| RSAD2, IFI27, OAS1, IFI44L, SIGLEC1, | 0.877 | 0.867 | 0.886 | 0.927 | 0.872 | 0.982 | 0.948 | 0.886 | 1 |
| ISG15, IL1R2, OLAH, FAM20A, RETN | | | | | | | | | |
| IFI27, IFIT1, SIGLEC1, ISG15, HERC6, | 0.877 | 0.866 | 0.887 | 0.941 | 0.895 | 0.986 | 0.951 | 0.889 | 1 |
| SLC1A2, OLAH, FAM20A, RETN, MMP8 | | | | | | | | | |
| RSAD2, IFI27, OAS1, IFI44L, SIGLEC1, | 0.877 | 0.866 | 0.887 | 0.935 | 0.886 | 0.985 | 0.940 | 0.874 | 1 |
| HERC6, SLC1A2, OLAH, FAM20A, MMP8 | | | | | | | | | |
| IFI27, OAS1, IFIT1, IFI44L, SIGLEC1, | 0.877 | 0.867 | 0.886 | 0.938 | 0.893 | 0.983 | 0.950 | 0.880 | 1 |
| SLC1A2, IL1R2, FAM20A, RETN, MMP8 | | | | | | | | | |
| RSAD2, IFI27, IFIT1, IFI44L, SIGLEC1, | 0.877 | 0.866 | 0.887 | 0.931 | 0.875 | 0.987 | 0.954 | 0.900 | 1 |
| ISG15, OLAH, FAM20A, RETN, MMP8 | | | | | | | | | |
| RSAD2, IFI27, OAS1, IFIT1, IFI44L, ISG15, | 0.877 | 0.866 | 0.887 | 0.930 | 0.876 | 0.984 | 0.933 | 0.865 | 1 |
| HERC6, OLAH, FAM20A, MMP8 | | | | | | | | | |
| RSAD2, IFI27, OAS1, IFIT1, SIGLEC1, | 0.876 | 0.866 | 0.887 | 0.938 | 0.891 | 0.984 | 0.936 | 0.867 | 1 |
| ISG15, HERC6, SLC1A2, OLAH, FAM20A | | | | | | | | | |
| IFI27, OAS1, IFIT1, IFI44L, SIGLEC1, | 0.876 | 0.866 | 0.887 | 0.937 | 0.891 | 0.984 | 0.936 | 0.868 | 1 |
| HERC6, SLC1A2, IL1R2, OLAH, FAM20A | | | | | | | | | |
| RSAD2, IFI27, OAS1, IFIT1, IFI44L, | 0.876 | 0.867 | 0.886 | 0.926 | 0.86 | 0.984 | 0.943 | 0.882 | 1 |
| SIGLEC1, ISG15, OLAH, FAM20A, RETN | | | | | | | | | |
| IFI27, OAS1, IFIT1, SIGLEC1, ISG15, | 0.876 | 0.867 | 0.886 | 0.939 | 0.896 | 0.982 | 0.949 | 0.879 | 1 |
| SLC1A2, IL1R2, FAM20A, RETN, MMP8 | | | | | | | | | |
| IFI27, IFIT1, SIGLEC1, HERC6, SLC1A2, | 0.876 | 0.866 | 0.886 | 0.940 | 0.894 | 0.986 | 0.952 | 0.886 | 1 |
| IL1R2, OLAH, FAM20A, RETN, MMP8 | | | | | | | | | |
| IFI27, OAS1, SIGLEC1, ISG15, HERC6, | 0.876 | 0.866 | 0.886 | 0.938 | 0.894 | 0.983 | 0.949 | 0.878 | 1 |
| SLC1A2, IL1R2, FAM20A, RETN, MMP8 | | | | | | | | | |
| RSAD2, IFI27, IFIT1, SIGLEC1, ISG15, | 0.876 | 0.867 | 0.886 | 0.938 | 0.896 | 0.981 | 0.952 | 0.883 | 1 |
| HERC6, SLC1A2, IL1R2, FAM20A, MMP8 | | | | | | | | | |
| RSAD2, IFI27, OAS1, IFIT1, IFI44L, | 0.876 | 0.866 | 0.886 | 0.926 | 0.870 | 0.983 | 0.948 | 0.885 | 1 |
| SIGLEC1, IL1R2, OLAH, FAM20A, RETN | | | | | | | | | |
| RSAD2, IFI27, IFIT1, IFI44L, SIGLEC1, | 0.876 | 0.866 | 0.886 | 0.938 | 0.894 | 0.982 | 0.953 | 0.885 | 1 |
| HERC6, SLC1A2, IL1R2, FAM20A, MMP8 | | | | | | | | | |
| IFI27, OAS1, IFIT1, SIGLEC1, ISG15, | 0.876 | 0.866 | 0.886 | 0.939 | 0.895 | 0.984 | 0.930 | 0.859 | 1 |
| HERC6, SLC1A2, OLAH, FAM20A, RETN | | | | | | | | | |
| RSAD2, IFI27, OAS1, IFIT1, SIGLEC1, | 0.876 | 0.866 | 0.886 | 0.936 | 0.892 | 0.980 | 0.950 | 0.882 | 1 |
| HERC6, SLC1A2, IL1R2, FAM20A, MMP8 | | | | | | | | | |
| IFI27, IFIT1, IFI44L, SIGLEC1, ISG15, | 0.876 | 0.865 | 0.887 | 0.937 | 0.889 | 0.986 | 0.949 | 0.885 | 1 |
| SLC1A2, OLAH, FAM20A, RETN, MMP8 | | | | | | | | | |
| IFI27, OAS1, IFI44L, SIGLEC1, HERC6, | 0.876 | 0.865 | 0.886 | 0.939 | 0.894 | 0.984 | 0.947 | 0.876 | 1 |
| SLC1A2, ILIR2, FAM20A, RETN, MMP8 | | | | | | | | | |
| RSAD2, IFI27, IFIT1, IFI44L, SIGLEC1, | 0.876 | 0.866 | 0.886 | 0.93 | 0.879 | 0.984 | 0.946 | 0.878 | 1 |
| ISG15, HERC6, IL1R2, FAM20A, RETN | | | | | | | | | |
| IFI27, IFIT1, IFI44L, ISG15, HERC6, IL1R2, | 0.876 | 0.865 | 0.887 | 0.932 | 0.879 | 0.985 | 0.934 | 0.862 | 1 |
| OLAH, FAM20A, RETN, MMP8 | | | | | | | | | |
| RSAD2, IFI27, OAS1, IFIT1, IFI44L, ISG15, | 0.876 | 0.866 | 0.886 | 0.930 | 0.877 | 0.983 | 0.928 | 0.855 | 1 |
| HERC6, IL1R2, OLAH, FAM20A | | | | | | | | | |
| IFI27, OAS1, IFI44L, SIGLEC1, ISG15, | 0.876 | 0.865 | 0.886 | 0.939 | 0.896 | 0.981 | 0.950 | 0.880 | 1 |
| HERC6, SLC1A2, IL1R2, FAM20A, MMP8 | | | | | | | | | |
| IFI27, IFIT1, IFI44L, SIGLEC1, ISG15, | 0.876 | 0.866 | 0.885 | 0.939 | 0.894 | 0.984 | 0.950 | 0.881 | 1 |
| SLC1A2, IL1R2, FAM20A, RETN, MMP8 | | | | | | | | | |
| IFI27, OAS1, IFIT1, SIGLEC1, SLC1A2, | 0.876 | 0.865 | 0.887 | 0.936 | 0.889 | 0.984 | 0.952 | 0.890 | 1 |
| IL1R2, OLAH, FAM20A, RETN, MMP8 | | | | | | | | | |
| IFI27, OAS1, IFIT1, SIGLEC1, ISG15, | 0.876 | 0.864 | 0.887 | 0.939 | 0.896 | 0.981 | 0.952 | 0.883 | 1 |
| HERC6, SLC1A2, IL1R2, FAM20A, MMP8 | | | | | | | | | |
| RSAD2, IFI27, OAS1, SIGLEC1, ISG15, | 0.876 | 0.866 | 0.886 | 0.937 | 0.890 | 0.983 | 0.935 | 0.864 | 1 |
| HERC6, SLC1A2, IL1R2, OLAH, FAM20A | | | | | | | | | |
| IFI27, IFIT1, IFI44L, SIGLEC1, HERC6, | 0.876 | 0.865 | 0.886 | 0.938 | 0.889 | 0.986 | 0.951 | 0.887 | 1 |
| SLC1A2, OLAH, FAM20A, RETN, MMP8 | | | | | | | | | |
| RSAD2, IFI27, IFIT1, SIGLEC1, ISG15, | 0.876 | 0.866 | 0.885 | 0.939 | 0.895 | 0.984 | 0.934 | 0.865 | 1 |
| HERC6, SLC1A2, IL1R2, OLAH, FAM20A | | | | | | | | | |
| IFI27, OAS1, IFIT1, SIGLEC1, HERC6, | 0.876 | 0.865 | 0.886 | 0.939 | 0.894 | 0.984 | 0.936 | 0.869 | 1 |
| SLC1A2, ILIR2, OLAH, FAM20A, RETN | | | | | | | | | |
| RSAD2, IFI27, IFIT1, IFI44L, SIGLEC1, | 0.875 | 0.865 | 0.886 | 0.937 | 0.891 | 0.983 | 0.933 | 0.864 | 1 |
| ISG15, HERC6, SLC1A2, OLAH, FAM20A | | | | | | | | | |
| RSAD2, IFI27, IFIT1, IFI44L, SIGLEC1, | 0.875 | 0.865 | 0.885 | 0.936 | 0.889 | 0.983 | 0.940 | 0.876 | 1 |
| HERC6, SLC1A2, IL1R2, OLAH, FAM20A | | | | | | | | | |
| RSAD2, IFI27, SIGLEC1, ISG15, HERC6, | 0.875 | 0.865 | 0.886 | 0.941 | 0.895 | 0.987 | 0.942 | 0.876 | 1 |
| SLC1A2, OLAH, FAM20A, RETN, MMP8 | | | | | | | | | |
| RSAD2, IFI27, OAS1, IFI44L, SIGLEC1, | 0.875 | 0.864 | 0.886 | 0.937 | 0.888 | 0.986 | 0.940 | 0.873 | 1 |
| ISG15, SLC1A2, OLAH, FAM20A, MMP8 | | | | | | | | | |
| RSAD2, IFI27, IFI44L, SIGLEC1, ISG15, | 0.875 | 0.864 | 0.887 | 0.936 | 0.883 | 0.989 | 0.948 | 0.886 | 1 |
| HERC6, OLAH, FAM20A, RETN, MMP8 | | | | | | | | | |
| IFI27, OAS1, IFIT1, IFI44L, SIGLEC1, | 0.875 | 0.864 | 0.887 | 0.934 | 0.883 | 0.985 | 0.950 | 0.889 | 1 |
| SLC1A2, OLAH, FAM20A, RETN, MMP8 | | | | | | | | | |
| RSAD2, IFI27, OAS1, IFI44L, SIGLEC1, | 0.875 | 0.865 | 0.886 | 0.937 | 0.890 | 0.984 | 0.948 | 0.884 | 1 |
| SLC1A2, IL1R2, OLAH, FAM20A, MMP8 | | | | | | | | | |
| IFI27, IFIT1, IFI44L, SIGLEC1, ISG15, | 0.875 | 0.866 | 0.885 | 0.938 | 0.892 | 0.985 | 0.936 | 0.868 | 1 |
| HERC6, SLC1A2, OLAH, FAM20A, RETN | | | | | | | | | |
| RSAD2, IFI27, IFIT1, SIGLEC1, ISG15, | 0.875 | 0.866 | 0.885 | 0.939 | 0.895 | 0.984 | 0.950 | 0.880 | 1 |
| SLC1A2, IL1R2, FAM20A, RETN, MMP8 | | | | | | | | | |
| RSAD2, IFI27, IFIT1, IFI44L, SIGLEC1, | 0.875 | 0.865 | 0.885 | 0.930 | 0.875 | 0.985 | 0.943 | 0.882 | 1 |
| ISG15, IL1R2, OLAH, FAM20A, RETN | | | | | | | | | |
| RSAD2, IFI27, OAS1, IFI44L, SIGLEC1, | 0.875 | 0.865 | 0.886 | 0.934 | 0.883 | 0.986 | 0.949 | 0.879 | 1 |
| ISG15, IL1R2, FAM20A, RETN, MMP8 | | | | | | | | | |
| IFI27, OAS1, IFI44L, SIGLEC1, HERC6, | 0.875 | 0.864 | 0.886 | 0.937 | 0.889 | 0.986 | 0.948 | 0.885 | 1 |
| SLC1A2, OLAH, FAM20A, RETN, MMP8 | | | | | | | | | |
| IFI27, IFIT1, IFI44L, SIGLEC1, SLC1A2, | 0.875 | 0.865 | 0.886 | 0.939 | 0.893 | 0.985 | 0.950 | 0.883 | 1 |
| IL1R2, OLAH, FAM20A, RETN, MMP8 | | | | | | | | | |
| RSAD2, IFI27, OAS1, IFIT1, IFI44L, | 0.875 | 0.865 | 0.885 | 0.933 | 0.881 | 0.984 | 0.940 | 0.875 | 1 |
| SIGLEC1, ISG15, SLC1A2, OLAH, FAM20A | | | | | | | | | |
| RSAD2, IFI27, IFI44L, SIGLEC1, ISG15, | 0.875 | 0.864 | 0.886 | 0.939 | 0.892 | 0.986 | 0.943 | 0.879 | 1 |
| HERC6, SLC1A2, OLAH, FAM20A, MMP8 | | | | | | | | | |
| RSAD2, IFI27, OAS1, SIGLEC1, HERC6, | 0.875 | 0.865 | 0.886 | 0.937 | 0.891 | 0.983 | 0.949 | 0.880 | 1 |
| SLC1A2, IL1R2, FAM20A, RETN, MMP8 | | | | | | | | | |
| RSAD2, IFI27, IFIT1, SIGLEC1, SLC1A2, | 0.875 | 0.865 | 0.885 | 0.939 | 0.892 | 0.985 | 0.950 | 0.886 | 1 |
| IL1R2, OLAH, FAM20A, RETN, MMP8 | | | | | | | | | |
| RSAD2, IFI27, OAS1, IFIT1, IFI44L, ISG15, | 0.875 | 0.864 | 0.886 | 0.929 | 0.876 | 0.982 | 0.935 | 0.867 | 1 |
| IL1R2, OLAH, FAM20A, MMP8 | | | | | | | | | |
| IFI27, OAS1, IFIT1, IFI44L, SIGLEC1, | 0.875 | 0.865 | 0.885 | 0.933 | 0.882 | 0.985 | 0.937 | 0.870 | 1 |
| ISG15, SLC1A2, OLAH, FAM20A, RETN | | | | | | | | | |
| IFI27, OAS1, IFIT1, IFI44L, SIGLEC1, | 0.875 | 0.865 | 0.885 | 0.935 | 0.885 | 0.985 | 0.941 | 0.872 | 1 |
| HERC6, SLC1A2, FAM20A, RETN, MMP8 | | | | | | | | | |
| IFI27, OAS1, IFI44L, SIGLEC1, ISG15, | 0.875 | 0.865 | 0.885 | 0.938 | 0.892 | 0.983 | 0.935 | 0.865 | 1 |
| HERC6, SLC1A2, IL1R2, OLAH, FAM20A | | | | | | | | | |
| RSAD2, IFI27, IFI44L, ISG15, HERC6, | 0.875 | 0.863 | 0.887 | 0.933 | 0.880 | 0.985 | 0.933 | 0.862 | 1 |
| IL1R2, OLAH, FAM20A, RETN, MMP8 | | | | | | | | | |
| RSAD2, IFI27, OAS1, IFIT1, IFI44L, ISG15, | 0.875 | 0.865 | 0.885 | 0.931 | 0.879 | 0.984 | 0.926 | 0.852 | 1 |
| HERC6, OLAH, FAM20A, RETN | | | | | | | | | |
| IFI27, IFIT1, SIGLEC1, ISG15, HERC6, | 0.875 | 0.865 | 0.885 | 0.940 | 0.896 | 0.984 | 0.953 | 0.885 | 1 |
| SLC1A2, IL1R2, FAM20A, RETN, MMP8 | | | | | | | | | |
| RSAD2, IFI27, OAS1, SIGLEC1, SLC1A2, | 0.875 | 0.864 | 0.885 | 0.937 | 0.889 | 0.985 | 0.948 | 0.883 | 1 |
| IL1R2, OLAH, FAM20A, RETN, MMP8 | | | | | | | | | |
| RSAD2, IFI27, IFI44L, SIGLEC1, HERC6, | 0.875 | 0.864 | 0.885 | 0.938 | 0.890 | 0.986 | 0.948 | 0.883 | 1 |
| SLC1A2, OLAH, FAM20A, RETN, MMP8 | | | | | | | | | |
| RSAD2, IFI27, SIGLEC1, ISG15, HERC6, | 0.875 | 0.865 | 0.885 | 0.937 | 0.890 | 0.983 | 0.950 | 0.880 | 1 |
| SLC1A2, ILIR2, FAM20A, RETN, MMP8 | | | | | | | | | |
| RSAD2, IFI27, OAS1, IFIT1, IFI44L, | 0.875 | 0.865 | 0.885 | 0.934 | 0.888 | 0.980 | 0.952 | 0.885 | 1 |
| SIGLEC1, SLC1A2, IL1R2, FAM20A, | | | | | | | | | |
| MMP8 | | | | | | | | | |
| IFI27, OAS1, IFIT1, IFI44L, ISG15, HERC6, | 0.875 | 0.865 | 0.885 | 0.930 | 0.877 | 0.982 | 0.927 | 0.853 | 1 |
| IL1R2, OLAH, FAM20A, RETN | | | | | | | | | |
| IFI27, OAS1, IFIT1, IFI44L, SIGLEC1, | 0.875 | 0.865 | 0.884 | 0.935 | 0.890 | 0.981 | 0.936 | 0.863 | 1 |
| HERC6, SLC1A2, IL1R2, FAM20A, RETN | | | | | | | | | |
| RSAD2, IFI27, OAS1, IFIT1, SIGLEC1, | 0.875 | 0.865 | 0.885 | 0.938 | 0.892 | 0.984 | 0.936 | 0.869 | 1 |
| HERC6, SLC1A2, OLAH, FAM20A, RETN | | | | | | | | | |
| RSAD2, IFI27, OAS1, IFI44L, SIGLEC1, | 0.875 | 0.864 | 0.885 | 0.936 | 0.888 | 0.984 | 0.937 | 0.867 | 1 |
| ISG15, HERC6, SLC1A2, OLAH, FAM20A | | | | | | | | | |
| IFI27, OAS1, IFIT1, IFI44L, SIGLEC1, | 0.875 | 0.865 | 0.884 | 0.937 | 0.893 | 0.981 | 0.942 | 0.873 | 1 |
| ISG15, HERC6, SLC1A2, IL1R2, FAM20A | | | | | | | | | |
| RSAD2, IFI27, OAS1, IFI44L, SIGLEC1, | 0.875 | 0.863 | 0.886 | 0.931 | 0.876 | 0.987 | 0.950 | 0.891 | 1 |
| ISG15, OLAH, FAM20A, RETN, MMP8 | | | | | | | | | |
| RSAD2, IFI27, OAS1, SIGLEC1, ISG15, | 0.875 | 0.864 | 0.885 | 0.937 | 0.893 | 0.981 | 0.948 | 0.879 | 1 |
| HERC6, SLC1A2, IL1R2, FAM20A, MMP8 | | | | | | | | | |
| IFI27, OAS1, IFIT1, IFI44L, SIGLEC1, | 0.874 | 0.865 | 0.884 | 0.935 | 0.886 | 0.985 | 0.937 | 0.870 | 1 |
| ISG15, SLC1A2, IL1R2, OLAH, FAM20A | | | | | | | | | |
| RSAD2, IFI27, IFI44L, ISG15, HERC6, | 0.874 | 0.864 | 0.885 | 0.937 | 0.891 | 0.983 | 0.925 | 0.849 | 1 |
| SLC1A2, IL1R2, OLAH, FAM20A, MMP8 | | | | | | | | | |
| IFI27, IFI44L, SIGLEC1, ISG15, HERC6, | 0.874 | 0.864 | 0.885 | 0.937 | 0.888 | 0.985 | 0.942 | 0.872 | 1 |
| SLC1A2, OLAH, FAM20A, RETN, MMP8 | | | | | | | | | |
| IFI27, OAS1, IFIT1, SIGLEC1, ISG15, | 0.874 | 0.864 | 0.884 | 0.936 | 0.887 | 0.985 | 0.937 | 0.869 | 1 |
| SLC1A2, IL1R2, OLAH, FAM20A, RETN | | | | | | | | | |
| IFI27, OAS1, IFIT1, IFI44L, HERC6, | 0.874 | 0.864 | 0.885 | 0.934 | 0.886 | 0.983 | 0.923 | 0.842 | 1 |
| SLC1A2, IL1R2, OLAH, FAM20A, MMP8 | | | | | | | | | |
| IFI27, OAS1, IFIT1, SIGLEC1, ISG15, | 0.874 | 0.864 | 0.885 | 0.935 | 0.885 | 0.985 | 0.940 | 0.869 | 1 |
| HERC6, SLC1A2, FAM20A, RETN, MMP8 | | | | | | | | | |
| RSAD2, IFI27, IFI44L, SIGLEC1, ISG15, | 0.874 | 0.864 | 0.884 | 0.938 | 0.895 | 0.981 | 0.952 | 0.883 | 1 |
| HERC6, SLC1A2, IL1R2, FAM20A, MMP8 | | | | | | | | | |
| IFI27, OAS1, IFI44L, ISG15, HERC6, | 0.874 | 0.863 | 0.886 | 0.937 | 0.890 | 0.983 | 0.922 | 0.841 | 1 |
| SLC1A2, IL1R2, OLAH, FAM20A, MMP8 | | | | | | | | | |
| IFI27, OAS1, IFI44L, SIGLEC1, ISG15, | 0.874 | 0.863 | 0.886 | 0.935 | 0.885 | 0.985 | 0.943 | 0.878 | 1 |
| SLC1A2, OLAH, FAM20A, RETN, MMP8 | | | | | | | | | |
| RSAD2, IFI27, OAS1, IFIT1, ISG15, HERC6, | 0.874 | 0.865 | 0.884 | 0.930 | 0.879 | 0.982 | 0.928 | 0.855 | 1 |
| IL1R2, OLAH, FAM20A, RETN | | | | | | | | | |
| RSAD2, IFI27, OAS1, IFIT1, IFI44L, | 0.874 | 0.864 | 0.884 | 0.931 | 0.879 | 0.983 | 0.927 | 0.853 | 1 |
| HERC6, IL1R2, OLAH, FAM20A, RETN | | | | | | | | | |
| IFI27, OAS1, IFIT1, IFI44L, SIGLEC1, | 0.874 | 0.865 | 0.883 | 0.928 | 0.875 | 0.980 | 0.951 | 0.900 | 1 |
| HERC6, IL1R2, OLAH, RETN, MMP8 | | | | | | | | | |
| IFI27, IFIT1, IFI44L, SIGLEC1, HERC6, | 0.874 | 0.864 | 0.884 | 0.937 | 0.889 | 0.984 | 0.941 | 0.875 | 1 |
| SLC1A2, ILIR2, OLAH, FAM20A, RETN | | | | | | | | | |
| IFI27, OAS1, IFIT1, IFI44L, SIGLEC1, | 0.874 | 0.864 | 0.884 | 0.937 | 0.894 | 0.981 | 0.951 | 0.882 | 1 |
| ISG15, SLC1A2, IL1R2, FAM20A, MMP8 | | | | | | | | | |
| RSAD2, IFI27, OAS1, IFIT1, SIGLEC1, | 0.874 | 0.864 | 0.884 | 0.935 | 0.885 | 0.985 | 0.940 | 0.869 | 1 |
| HERC6, SLC1A2, FAM20A, RETN, MMP8 | | | | | | | | | |
| IFI27, OAS1, IFIT1, SIGLEC1, ISG15, | 0.874 | 0.865 | 0.883 | 0.927 | 0.874 | 0.981 | 0.958 | 0.909 | 1 |
| HERC6, IL1R2, OLAH, RETN, MMP8 | | | | | | | | | |
| RSAD2, IFI27, OAS1, IFI44L, ISG15, | 0.874 | 0.862 | 0.886 | 0.931 | 0.877 | 0.986 | 0.928 | 0.855 | 1 |
| HERC6, OLAH, FAM20A, RETN, MMP8 | | | | | | | | | |
| IFI27, OAS1, IFIT1, IFI44L, ISG15, IL1R2, | 0.874 | 0.863 | 0.885 | 0.928 | 0.873 | 0.983 | 0.934 | 0.864 | 1 |
| OLAH, FAM20A, RETN, MMP8 | | | | | | | | | |
| RSAD2, IFI27, OAS1, IFI44L, ISG15, | 0.874 | 0.863 | 0.885 | 0.932 | 0.880 | 0.983 | 0.929 | 0.856 | 1 |
| HERC6, IL1R2, OLAH, FAM20A, RETN | | | | | | | | | |
| IFI27, IFIT1, IFI44L, SIGLEC1, ISG15, | 0.874 | 0.864 | 0.884 | 0.936 | 0.888 | 0.984 | 0.936 | 0.868 | 1 |
| SLC1A2, IL1R2, OLAH, FAM20A, RETN | | | | | | | | | |
| IFI27, OAS1, IFIT1, IFI44L, HERC6, | 0.874 | 0.863 | 0.885 | 0.937 | 0.888 | 0.985 | 0.921 | 0.837 | 1 |
| SLC1A2, OLAH, FAM20A, RETN, MMP8 | | | | | | | | | |
| RSAD2, IFI27, OAS1, IFIT1, SIGLEC1, | 0.874 | 0.864 | 0.884 | 0.935 | 0.887 | 0.983 | 0.941 | 0.875 | 1 |
| ISG15, SLC1A2, IL1R2, OLAH, FAM20A | | | | | | | | | |
| IFI27, IFI44L, SIGLEC1, HERC6, SLC1A2, | 0.874 | 0.864 | 0.884 | 0.939 | 0.892 | 0.986 | 0.952 | 0.888 | 1 |
| IL1R2, OLAH, FAM20A, RETN, MMP8 | | | | | | | | | |
| RSAD2, IFI27, IFI44L, SIGLEC1, ISG15, | 0.874 | 0.864 | 0.884 | 0.937 | 0.891 | 0.983 | 0.936 | 0.867 | 1 |
| HERC6, SLC1A2, IL1R2, OLAH, FAM20A | | | | | | | | | |
| RSAD2, IFI27, OAS1, IFIT1, IFI44L, | 0.874 | 0.865 | 0.883 | 0.927 | 0.874 | 0.981 | 0.947 | 0.889 | 1 |
| SIGLEC1, HERC6, OLAH, RETN, MMP8 | | | | | | | | | |
| RSAD2, IFI27, IFIT1, IFI44L, SIGLEC1, | 0.874 | 0.863 | 0.885 | 0.935 | 0.886 | 0.985 | 0.950 | 0.889 | 1 |
| SLC1A2, OLAH, FAM20A, RETN, MMP8 | | | | | | | | | |
| RSAD2, IFI27, IFI44L, SIGLEC1, HERC6, | 0.874 | 0.863 | 0.885 | 0.938 | 0.890 | 0.985 | 0.948 | 0.877 | 1 |
| SLC1A2, IL1R2, FAM20A, RETN, MMP8 | | | | | | | | | |
| RSAD2, IFI27, OAS1, IFIT1, SIGLEC1, | 0.874 | 0.864 | 0.884 | 0.937 | 0.894 | 0.980 | 0.951 | 0.882 | 1 |
| ISG15, SLC1A2, IL1R2, FAM20A, MMP8 | | | | | | | | | |
| RSAD2, IFI27, OAS1, IFIT1, IFI44L, | 0.874 | 0.863 | 0.885 | 0.931 | 0.881 | 0.982 | 0.922 | 0.840 | 1 |
| HERC6, SLC1A2, OLAH, FAM20A, MMP8 | | | | | | | | | |
| RSAD2, IFI27, OAS1, IFIT1, HERC6, | 0.874 | 0.864 | 0.884 | 0.933 | 0.885 | 0.981 | 0.923 | 0.842 | 1 |
| SLC1A2, IL1R2, OLAH, FAM20A, MMP8 | | | | | | | | | |
| RSAD2, IFI27, OAS1, IFIT1, ISG15, IL1R2, | 0.874 | 0.863 | 0.884 | 0.927 | 0.872 | 0.983 | 0.933 | 0.862 | 1 |
| OLAH, FAM20A, RETN, MMP8 | | | | | | | | | |
| RSAD2, IFI27, OAS1, SIGLEC1, ISG15, | 0.874 | 0.863 | 0.885 | 0.936 | 0.887 | 0.986 | 0.946 | 0.882 | 1 |
| SLC1A2, OLAH, FAM20A, RETN, MMP8 | | | | | | | | | |
| RSAD2, IFI27, IFIT1, IFI44L, SIGLEC1, | 0.874 | 0.864 | 0.883 | 0.937 | 0.889 | 0.984 | 0.937 | 0.869 | 1 |
| ISG15, SLC1A2, IL1R2, OLAH, FAM20A | | | | | | | | | |
| RSAD2, IFI27, OAS1, IFI44L, SIGLEC1, | 0.874 | 0.863 | 0.884 | 0.934 | 0.884 | 0.984 | 0.943 | 0.875 | 1 |
| ISG15, HERC6, IL1R2, FAM20A, RETN | | | | | | | | | |
| IFI27, OAS1, IFI44L, SIGLEC1, ISG15, | 0.874 | 0.863 | 0.884 | 0.936 | 0.889 | 0.984 | 0.937 | 0.867 | 1 |
| HERC6, SLC1A2, OLAH, FAM20A, RETN | | | | | | | | | |
| RSAD2, IFI27, IFIT1, IFI44L, SIGLEC1, | 0.874 | 0.864 | 0.883 | 0.938 | 0.896 | 0.981 | 0.952 | 0.883 | 1 |
| ISG15, SLC1A2, IL1R2, FAM20A, MMP8 | | | | | | | | | |
| RSAD2, IFI27, OAS1, IFIT1, SIGLEC1, | 0.874 | 0.864 | 0.883 | 0.927 | 0.874 | 0.981 | 0.958 | 0.909 | 1 |
| HERC6, IL1R2, OLAH, RETN, MMP8 | | | | | | | | | |
| RSAD2, IFI27, OAS1, IFIT1, SIGLEC1, | 0.874 | 0.863 | 0.885 | 0.933 | 0.882 | 0.985 | 0.952 | 0.894 | 1 |
| SLC1A2, OLAH, FAM20A, RETN, MMP8 | | | | | | | | | |
| RSAD2, IFI27, IFIT1, HERC6, SLC1A2, | 0.874 | 0.864 | 0.884 | 0.933 | 0.884 | 0.983 | 0.923 | 0.843 | 1 |
| IL1R2, OLAH, FAM20A, RETN, MMP8 | | | | | | | | | |
| IFI27, OAS1, IFIT1, IFI44L, ISG15, HERC6, | 0.874 | 0.863 | 0.884 | 0.932 | 0.880 | 0.984 | 0.929 | 0.848 | 1 |
| IL1R2, FAM20A, RETN, MMP8 | | | | | | | | | |
| IFI27, OAS1, IFIT1, SIGLEC1, ISG15, | 0.874 | 0.863 | 0.884 | 0.936 | 0.891 | 0.981 | 0.938 | 0.866 | 1 |
| HERC6, SLC1A2, ILIR2, FAM20A, RETN | | | | | | | | | |
| IFI27, IFI44L, SIGLEC1, ISG15, HERC6, | 0.874 | 0.863 | 0.884 | 0.938 | 0.893 | 0.983 | 0.951 | 0.882 | 1 |
| SLC1A2, IL1R2, FAM20A, RETN, MMP8 | | | | | | | | | |
| RSAD2, IFI27, IFIT1, IFI44L, SIGLEC1, | 0.873 | 0.863 | 0.884 | 0.938 | 0.892 | 0.985 | 0.938 | 0.872 | 1 |
| HERC6, SLC1A2, OLAH, FAM20A, RETN | | | | | | | | | |
| RSAD2, IFI27, IFIT1, IFI44L, ISG15, | 0.873 | 0.862 | 0.885 | 0.929 | 0.874 | 0.984 | 0.932 | 0.861 | 1 |
| HERC6, OLAH, FAM20A, RETN, MMP8 | | | | | | | | | |
| RSAD2, IFI27, OAS1, IFIT1, IFI44L, IL1R2, | 0.873 | 0.863 | 0.884 | 0.929 | 0.874 | 0.983 | 0.932 | 0.861 | 1 |
| OLAH, FAM20A, RETN, MMP8 | | | | | | | | | |
| IFI27, OAS1, IFIT1, IFI44L, ISG15, HERC6, | 0.873 | 0.862 | 0.885 | 0.931 | 0.881 | 0.982 | 0.922 | 0.841 | 1 |
| SLC1A2, OLAH, FAM20A, MMP8 | | | | | | | | | |
| IFI27, OAS1, SIGLEC1, ISG15, HERC6, | 0.873 | 0.863 | 0.884 | 0.935 | 0.888 | 0.983 | 0.935 | 0.864 | 1 |
| SLC1A2, ILIR2, OLAH, FAM20A, RETN | | | | | | | | | |
| IFI27, OAS1, IFI44L, SIGLEC1, SLC1A2, | 0.873 | 0.863 | 0.884 | 0.935 | 0.887 | 0.984 | 0.949 | 0.885 | 1 |
| IL1R2, OLAH, FAM20A, RETN, MMP8 | | | | | | | | | |
| IFI27, OAS1, IFIT1, HERC6, SLC1A2, | 0.873 | 0.863 | 0.884 | 0.936 | 0.888 | 0.984 | 0.920 | 0.836 | 1 |
| IL1R2, OLAH, FAM20A, RETN, MMP8 | | | | | | | | | |
| RSAD2, IFI27, IFI44L, SIGLEC1, SLC1A2, | 0.873 | 0.862 | 0.884 | 0.939 | 0.892 | 0.985 | 0.949 | 0.885 | 1 |
| IL1R2, OLAH, FAM20A, RETN, MMP8 | | | | | | | | | |
| IFI27, OAS1, IFIT1, IFI44L, SIGLEC1, | 0.873 | 0.863 | 0.883 | 0.932 | 0.881 | 0.984 | 0.939 | 0.873 | 1 |
| SLC1A2, IL1R2, OLAH, FAM20A, RETN | | | | | | | | | |
| RSAD2, IFI27, OAS1, IFI44L, ISG15, | 0.873 | 0.862 | 0.884 | 0.932 | 0.881 | 0.982 | 0.923 | 0.844 | 1 |
| HERC6, SLC1A2, OLAH, FAM20A, MMP8 | | | | | | | | | |
| RSAD2, IFI27, OAS1, SIGLEC1, ISG15, | 0.873 | 0.863 | 0.884 | 0.936 | 0.888 | 0.984 | 0.934 | 0.862 | 1 |
| HERC6, SLC1A2, OLAH, FAM20A, RETN | | | | | | | | | |
| IFI27, IFI44L, SIGLEC1, ISG15, HERC6, | 0.873 | 0.863 | 0.883 | 0.937 | 0.891 | 0.983 | 0.940 | 0.871 | 1 |
| SLC1A2, IL1R2, OLAH, FAM20A, RETN | | | | | | | | | |
| IFI27, OAS1, IFIT1, ISG15, HERC6, | 0.873 | 0.863 | 0.884 | 0.933 | 0.883 | 0.983 | 0.922 | 0.840 | 1 |
| SLC1A2, OLAH, FAM20A, RETN, MMP8 | | | | | | | | | |
| RSAD2, IFI27, OAS1, IFI44L, SIGLEC1, | 0.873 | 0.864 | 0.883 | 0.927 | 0.875 | 0.980 | 0.953 | 0.903 | 1 |
| HERC6, IL1R2, OLAH, RETN, MMP8 | | | | | | | | | |
| IFI27, OAS1, IFIT1, IFI44L, SIGLEC1, | 0.873 | 0.863 | 0.883 | 0.932 | 0.884 | 0.981 | 0.938 | 0.868 | 1 |
| ISG15, HERC6, SLC1A2, FAM20A, RETN | | | | | | | | | |
| RSAD2, IFI27, OAS1, IFI44L, SIGLEC1, | 0.873 | 0.862 | 0.884 | 0.932 | 0.878 | 0.986 | 0.942 | 0.869 | 1 |
| ISG15, HERC6, FAM20A, RETN, MMP8 | | | | | | | | | |
| RSAD2, IFI27, IFIT1, SIGLEC1, ISG15, | 0.873 | 0.863 | 0.883 | 0.937 | 0.889 | 0.985 | 0.937 | 0.870 | 1 |
| SLC1A2, IL1R2, OLAH, FAM20A, RETN | | | | | | | | | |
| RSAD2, IFI27, OAS1, IFIT1, SIGLEC1, | 0.873 | 0.863 | 0.883 | 0.937 | 0.894 | 0.980 | 0.941 | 0.871 | 1 |
| ISG15, HERC6, SLC1A2, IL1R2, FAM20A | | | | | | | | | |
| IFI27, IFI44L, ISG15, HERC6, SLC1A2, | 0.873 | 0.863 | 0.883 | 0.935 | 0.888 | 0.982 | 0.922 | 0.841 | 1 |
| IL1R2, OLAH, FAM20A, RETN, MMP8 | | | | | | | | | |
| IFI27, IFIT1, SIGLEC1, ISG15, HERC6, | 0.873 | 0.862 | 0.884 | 0.940 | 0.895 | 0.985 | 0.937 | 0.870 | 1 |
| SLC1A2, ILIR2, OLAH, FAM20A, RETN | | | | | | | | | |
| IFI27, OAS1, IFI44L, SIGLEC1, ISG15, | 0.873 | 0.863 | 0.883 | 0.938 | 0.894 | 0.983 | 0.947 | 0.876 | 1 |
| SLC1A2, IL1R2, FAM20A, RETN, MMP8 | | | | | | | | | |
| RSAD2, IFI27, IFIT1, IFI44L, HERC6, | 0.873 | 0.862 | 0.884 | 0.934 | 0.886 | 0.982 | 0.926 | 0.850 | 1 |
| SLC1A2, IL1R2, OLAH, FAM20A, MMP8 | | | | | | | | | |
| RSAD2, IFI27, IFIT1, IFI44L, ISG15, | 0.873 | 0.863 | 0.883 | 0.930 | 0.878 | 0.983 | 0.928 | 0.856 | 1 |
| HERC6, IL1R2, OLAH, FAM20A, RETN | | | | | | | | | |
| IFI27, OAS1, IFIT1, IFI44L, SIGLEC1, | 0.873 | 0.863 | 0.882 | 0.926 | 0.871 | 0.980 | 0.952 | 0.900 | 1 |
| ISG15, HERC6, OLAH, RETN, MMP8 | | | | | | | | | |
| RSAD2, IFI27, OAS1, SIGLEC1, ISG15, | 0.873 | 0.862 | 0.883 | 0.933 | 0.884 | 0.983 | 0.938 | 0.868 | 1 |
| SLC1A2, IL1R2, OLAH, FAM20A, RETN | | | | | | | | | |
| RSAD2, IFI27, IFIT1, ISG15, HERC6, | 0.873 | 0.863 | 0.883 | 0.933 | 0.886 | 0.981 | 0.926 | 0.849 | 1 |
| SLC1A2, IL1R2, OLAH, FAM20A, MMP8 | | | | | | | | | |
| IFI27, OAS1, IFI44L, SIGLEC1, ISG15, | 0.873 | 0.863 | 0.883 | 0.934 | 0.885 | 0.984 | 0.940 | 0.873 | 1 |
| SLC1A2, ILIR2, OLAH, FAM20A, RETN | | | | | | | | | |
| RSAD2, IFI27, IFIT1, SIGLEC1, ISG15, | 0.873 | 0.862 | 0.883 | 0.938 | 0.893 | 0.983 | 0.934 | 0.866 | 1 |
| HERC6, SLC1A2, OLAH, FAM20A, RETN | | | | | | | | | |
| RSAD2, IFI27, OAS1, IFIT1, IFI44L, | 0.873 | 0.863 | 0.883 | 0.933 | 0.882 | 0.984 | 0.940 | 0.874 | 1 |
| SIGLEC1, SLC1A2, IL1R2, OLAH, FAM20A | | | | | | | | | |
| RSAD2, IFI27, IFI44L, SIGLEC1, ISG15, | 0.873 | 0.863 | 0.883 | 0.934 | 0.886 | 0.982 | 0.935 | 0.865 | 1 |
| HERC6, SLC1A2, OLAH, FAM20A, RETN | | | | | | | | | |
| RSAD2, IFI27, OAS1, IFI44L, ISG15, IL1R2, | 0.873 | 0.861 | 0.884 | 0.930 | 0.875 | 0.984 | 0.930 | 0.860 | 1 |
| OLAH, FAM20A, RETN, MMP8 | | | | | | | | | |
| IFI27, OAS1, IFI44L, SIGLEC1, ISG15, | 0.873 | 0.863 | 0.882 | 0.927 | 0.875 | 0.980 | 0.951 | 0.900 | 1 |
| HERC6, IL1R2, OLAH, RETN, MMP8 | | | | | | | | | |
| RSAD2, IFI27, IFIT1, SIGLEC1, HERC6, | 0.873 | 0.863 | 0.883 | 0.938 | 0.893 | 0.984 | 0.939 | 0.873 | 1 |
| SLC1A2, IL1R2, OLAH, FAM20A, RETN | | | | | | | | | |
| RSAD2, IFI27, OAS1, IFIT1, HERC6, | 0.873 | 0.862 | 0.883 | 0.933 | 0.883 | 0.983 | 0.922 | 0.840 | 1 |
| SLC1A2, OLAH, FAM20A, RETN, MMP8 | | | | | | | | | |
| RSAD2, IFI27, IFIT1, IFI44L, ISG15, IL1R2, | 0.873 | 0.862 | 0.884 | 0.928 | 0.872 | 0.983 | 0.934 | 0.865 | 1 |
| OLAH, FAM20A, RETN, MMP8 | | | | | | | | | |
| IFI27, IFIT1, IFI44L, ISG15, HERC6, | 0.873 | 0.863 | 0.883 | 0.935 | 0.887 | 0.982 | 0.924 | 0.846 | 1 |
| SLC1A2, IL1R2, OLAH, FAM20A, MMP8 | | | | | | | | | |
| RSAD2, IFI27, OAS1, IFI44L, SIGLEC1, | 0.873 | 0.862 | 0.883 | 0.933 | 0.884 | 0.982 | 0.938 | 0.870 | 1 |
| HERC6, SLC1A2, IL1R2, OLAH, FAM20A | | | | | | | | | |
| RSAD2, IFI27, OAS1, IFI44L, SIGLEC1, | 0.873 | 0.861 | 0.884 | 0.937 | 0.892 | 0.981 | 0.947 | 0.878 | 1 |
| HERC6, SLC1A2, IL1R2, FAM20A, MMP8 | | | | | | | | | |
| RSAD2, IFI27, IFIT1, IFI44L, SIGLEC1, | 0.873 | 0.863 | 0.882 | 0.931 | 0.879 | 0.984 | 0.942 | 0.868 | 1 |
| HERC6, SLC1A2, FAM20A, RETN, MMP8 | | | | | | | | | |
| RSAD2, IFI27, OAS1, IFIT1, SIGLEC1, | 0.873 | 0.863 | 0.882 | 0.927 | 0.873 | 0.981 | 0.955 | 0.905 | 1 |
| ISG15, HERC6, OLAH, RETN, MMP8 | | | | | | | | | |
| RSAD2, IFI27, OAS1, IFIT1, IFI44L, | 0.873 | 0.862 | 0.883 | 0.936 | 0.893 | 0.980 | 0.941 | 0.872 | 1 |
| SIGLEC1, HERC6, SLC1A2, IL1R2, | | | | | | | | | |
| FAM20A | | | | | | | | | |
| RSAD2, IFI27, OAS1, HERC6, SLC1A2, | 0.872 | 0.862 | 0.883 | 0.934 | 0.885 | 0.983 | 0.922 | 0.839 | 1 |
| IL1R2, OLAH, FAM20A, RETN, MMP8 | | | | | | | | | |
| IFI27, OAS1, IFI44L, ISG15, HERC6, | 0.872 | 0.862 | 0.883 | 0.934 | 0.885 | 0.984 | 0.922 | 0.840 | 1 |
| SLC1A2, OLAH, FAM20A, RETN, MMP8 | | | | | | | | | |
| RSAD2, IFI27, OAS1, IFIT1, SIGLEC1, | 0.872 | 0.863 | 0.882 | 0.933 | 0.883 | 0.984 | 0.940 | 0.874 | 1 |
| ISG15, SLC1A2, OLAH, FAM20A, RETN | | | | | | | | | |
| RSAD2, IFI27, OAS1, IFI44L, SIGLEC1, | 0.872 | 0.862 | 0.883 | 0.936 | 0.889 | 0.983 | 0.943 | 0.875 | 1 |
| ISG15, SLC1A2, IL1R2, OLAH, FAM20A | | | | | | | | | |
| RSAD2, IFI27, OAS1, IFIT1, IFI44L, ISG15, | 0.872 | 0.862 | 0.883 | 0.930 | 0.877 | 0.983 | 0.923 | 0.846 | 1.000 |
| SLC1A2, OLAH, FAM20A, MMP8 | | | | | | | | | |
| RSAD2, IFI27, OAS1, SIGLEC1, ISG15, | 0.872 | 0.862 | 0.883 | 0.937 | 0.892 | 0.982 | 0.948 | 0.877 | 1 |
| SLC1A2, IL1R2, FAM20A, RETN, MMP8 | | | | | | | | | |
| RSAD2, IFI27, OAS1, IFIT1, IFI44L, | 0.872 | 0.862 | 0.883 | 0.931 | 0.878 | 0.984 | 0.940 | 0.873 | 1 |
| SIGLEC1, SLC1A2, OLAH, FAM20A, RETN | | | | | | | | | |
| RSAD2, IFI27, OAS1, IFIT1, IFI44L, | 0.872 | 0.862 | 0.883 | 0.929 | 0.875 | 0.982 | 0.949 | 0.886 | 1 |
| SIGLEC1, ISG15, IL1R2, FAM20A, RETN | | | | | | | | | |
| RSAD2, IFI27, OAS1, IFI44L, ISG15, | 0.872 | 0.862 | 0.883 | 0.935 | 0.887 | 0.982 | 0.917 | 0.833 | 1 |
| HERC6, SLC1A2, IL1R2, OLAH, FAM20A | | | | | | | | | |
| RSAD2, IFI27, OAS1, IFI44L, HERC6, | 0.872 | 0.861 | 0.884 | 0.935 | 0.888 | 0.983 | 0.923 | 0.843 | 1 |
| SLC1A2, IL1R2, OLAH, FAM20A, MMP8 | | | | | | | | | |
| RSAD2, IFI27, OAS1, IFIT1, SIGLEC1, | 0.872 | 0.862 | 0.883 | 0.939 | 0.894 | 0.983 | 0.948 | 0.876 | 1 |
| SLC1A2, IL1R2, FAM20A, RETN, MMP8 | | | | | | | | | |
| RSAD2, IFI27, OAS1, IFIT1, IFI44L, | 0.872 | 0.863 | 0.881 | 0.926 | 0.873 | 0.979 | 0.942 | 0.884 | 1 |
| SIGLEC1, HERC6, IL1R2, OLAH, RETN | | | | | | | | | |
| RSAD2, IFI27, OAS1, IFIT1, IFI44L, | 0.872 | 0.862 | 0.883 | 0.932 | 0.880 | 0.984 | 0.930 | 0.852 | 1 |
| HERC6, IL1R2, FAM20A, RETN, MMP8 | | | | | | | | | |
| RSAD2, IFI27, OAS1, IFIT1, IFI44L, | 0.872 | 0.864 | 0.881 | 0.925 | 0.871 | 0.979 | 0.941 | 0.883 | 1.000 |
| SIGLEC1, ISG15, HERC6, OLAH, RETN | | | | | | | | | |
| RSAD2, IFI27, OAS1, IFI44L, SIGLEC1, | 0.872 | 0.862 | 0.883 | 0.938 | 0.895 | 0.981 | 0.949 | 0.879 | 1 |
| ISG15, SLC1A2, IL1R2, FAM20A, MMP8 | | | | | | | | | |
| RSAD2, IFI27, OAS1, SIGLEC1, ISG15, | 0.872 | 0.862 | 0.882 | 0.927 | 0.872 | 0.981 | 0.955 | 0.905 | 1 |
| HERC6, IL1R2, OLAH, RETN, MMP8 | | | | | | | | | |
| RSAD2, IFI27, IFIT1, IFI44L, SIGLEC1, | 0.872 | 0.861 | 0.883 | 0.938 | 0.891 | 0.985 | 0.952 | 0.883 | 1 |
| SLC1A2, IL1R2, FAM20A, RETN, MMP8 | | | | | | | | | |
| RSAD2, IFI27, IFIT1, IFI44L, ISG15, | 0.872 | 0.862 | 0.883 | 0.935 | 0.887 | 0.982 | 0.925 | 0.848 | 1 |
| SLC1A2, IL1R2, OLAH, FAM20A, MMP8 | | | | | | | | | |
| IFI27, OAS1, IFI44L, SIGLEC1, HERC6, | 0.872 | 0.862 | 0.883 | 0.934 | 0.886 | 0.983 | 0.936 | 0.865 | 1 |
| SLC1A2, IL1R2, OLAH, FAM20A, RETN | | | | | | | | | |
| RSAD2, IFI27, OAS1, IFIT1, SIGLEC1, | 0.872 | 0.863 | 0.882 | 0.936 | 0.891 | 0.981 | 0.937 | 0.864 | 1 |
| HERC6, SLC1A2, IL1R2, FAM20A, RETN | | | | | | | | | |
| IFI27, OAS1, IFI44L, HERC6, SLC1A2, | 0.872 | 0.861 | 0.883 | 0.937 | 0.889 | 0.984 | 0.923 | 0.841 | 1 |
| IL1R2, OLAH, FAM20A, RETN, MMP8 | | | | | | | | | |
| RSAD2, IFI27, OAS1, ISG15, HERC6, | 0.872 | 0.861 | 0.883 | 0.934 | 0.886 | 0.981 | 0.920 | 0.838 | 1 |
| SLC1A2, IL1R2, OLAH, FAM20A, MMP8 | | | | | | | | | |
| RSAD2, IFI27, SIGLEC1, ISG15, HERC6, | 0.872 | 0.861 | 0.883 | 0.935 | 0.888 | 0.983 | 0.938 | 0.870 | 1 |
| SLC1A2, ILIR2, OLAH, FAM20A, RETN | | | | | | | | | |
| IFI27, OAS1, IFIT1, ISG15, HERC6, | 0.872 | 0.861 | 0.883 | 0.934 | 0.886 | 0.981 | 0.922 | 0.840 | 1 |
| SLC1A2, IL1R2, OLAH, FAM20A, MMP8 | | | | | | | | | |
| RSAD2, IFI27, OAS1, IFIT1, SIGLEC1, | 0.872 | 0.863 | 0.881 | 0.926 | 0.873 | 0.979 | 0.947 | 0.891 | 1 |
| ISG15, HERC6, IL1R2, OLAH, RETN | | | | | | | | | |
| RSAD2, IFI27, IFI44L, HERC6, SLC1A2, | 0.872 | 0.861 | 0.883 | 0.936 | 0.888 | 0.984 | 0.924 | 0.844 | 1 |
| IL1R2, OLAH, FAM20A, RETN, MMP8 | | | | | | | | | |
| RSAD2, IFI27, OAS1, IFIT1, IFI44L, | 0.872 | 0.862 | 0.882 | 0.933 | 0.885 | 0.982 | 0.920 | 0.837 | 1 |
| HERC6, SLC1A2, IL1R2, OLAH, FAM20A | | | | | | | | | |
| RSAD2, IFI27, IFIT1, IFI44L, SIGLEC1, | 0.872 | 0.862 | 0.882 | 0.935 | 0.889 | 0.981 | 0.949 | 0.880 | 1 |
| ISG15, HERC6, SLC1A2, IL1R2, FAM20A | | | | | | | | | |
| RSAD2, IFI27, IFIT1, IFI44L, SIGLEC1, | 0.872 | 0.862 | 0.882 | 0.935 | 0.885 | 0.985 | 0.939 | 0.873 | 1 |
| ISG15, SLC1A2, OLAH, FAM20A, RETN | | | | | | | | | |
| IFI27, IFIT1, IFI44L, ISG15, SLC1A2, | 0.872 | 0.862 | 0.882 | 0.933 | 0.885 | 0.982 | 0.923 | 0.843 | 1 |
| ILIR2, OLAH, FAM20A, RETN, MMP8 | | | | | | | | | |
| RSAD2, IFI27, IFIT1, SIGLEC1, ISG15, | 0.872 | 0.862 | 0.882 | 0.930 | 0.878 | 0.983 | 0.943 | 0.870 | 1 |
| HERC6, SLC1A2, FAM20A, RETN, MMP8 | | | | | | | | | |
| RSAD2, IFI27, IFIT1, IFI44L, HERC6, | 0.872 | 0.861 | 0.882 | 0.936 | 0.887 | 0.984 | 0.922 | 0.842 | 1 |
| SLC1A2, OLAH, FAM20A, RETN, MMP8 | | | | | | | | | |
| RSAD2, IFI27, OAS1, IFIT1, SIGLEC1, | 0.872 | 0.862 | 0.882 | 0.933 | 0.883 | 0.983 | 0.943 | 0.878 | 1 |
| SLC1A2, IL1R2, OLAH, FAM20A, RETN | | | | | | | | | |
| RSAD2, IFI27, IFIT1, IFI44L, SIGLEC1, | 0.872 | 0.863 | 0.881 | 0.926 | 0.873 | 0.980 | 0.952 | 0.903 | 1 |
| HERC6, IL1R2, OLAH, RETN, MMP8 | | | | | | | | | |
| IFI27, IFIT1, IFI44L, HERC6, SLC1A2, | 0.872 | 0.862 | 0.882 | 0.934 | 0.886 | 0.983 | 0.928 | 0.850 | 1 |
| IL1R2, OLAH, FAM20A, RETN, MMP8 | | | | | | | | | |
| RSAD2, IFI27, OAS1, IFI44L, ISG15, | 0.872 | 0.861 | 0.883 | 0.935 | 0.887 | 0.983 | 0.924 | 0.847 | 1 |
| SLC1A2, IL1R2, OLAH, FAM20A, MMP8 | | | | | | | | | |
| RSAD2, IFI27, OAS1, IFIT1, ISG15, | 0.872 | 0.861 | 0.882 | 0.934 | 0.886 | 0.981 | 0.924 | 0.845 | 1 |
| SLC1A2, IL1R2, OLAH, FAM20A, MMP8 | | | | | | | | | |
| RSAD2, OAS1, IFIT1, IFI44L, SIGLEC1, | 0.872 | 0.862 | 0.88 | 0.924 | 0.870 | 0.977 | 0.963 | 0.909 | 1 |
| HERC6, IL1R2, OLAH, FAM20A, MMP8 | | | | | | | | | |
| IFI27, IFIT1, IFI44L, SIGLEC1, ISG15, | 0.872 | 0.862 | 0.882 | 0.934 | 0.887 | 0.980 | 0.947 | 0.875 | 1 |
| HERC6, SLC1A2, IL1R2, FAM20A, RETN | | | | | | | | | |
| RSAD2, IFI27, IFIT1, IFI44L, ISG 15, | 0.872 | 0.861 | 0.883 | 0.933 | 0.884 | 0.983 | 0.923 | 0.845 | 1 |
| HERC6, SLC1A2, OLAH, FAM20A, MMP8 | | | | | | | | | |
| RSAD2, IFI27, OAS1, IFIT1, IFI44L, | 0.872 | 0.862 | 0.882 | 0.934 | 0.884 | 0.983 | 0.926 | 0.850 | 1 |
| SLC1A2, IL1R2, OLAH, FAM20A, MMP8 | | | | | | | | | |
| RSAD2, IFI27, ISG15, HERC6, SLC1A2, | 0.872 | 0.861 | 0.882 | 0.935 | 0.888 | 0.982 | 0.924 | 0.844 | 1 |
| IL1R2, OLAH, FAM20A, RETN, MMP8 | | | | | | | | | |
| RSAD2, IFI27, IFIT1, ISG15, HERC6, | 0.872 | 0.861 | 0.882 | 0.933 | 0.884 | 0.983 | 0.923 | 0.844 | 1 |
| SLC1A2, OLAH, FAM20A, RETN, MMP8 | | | | | | | | | |
| IFI27, OAS1, ISG15, HERC6, SLC1A2, | 0.872 | 0.861 | 0.882 | 0.935 | 0.888 | 0.982 | 0.920 | 0.836 | 1 |
| IL1R2, OLAH, FAM20A, RETN, MMP8 | | | | | | | | | |
| RSAD2, IFI27, OAS1, IFI44L, SIGLEC1, | 0.872 | 0.861 | 0.882 | 0.934 | 0.885 | 0.982 | 0.936 | 0.866 | 1 |
| HERC6, SLC1A2, OLAH, FAM20A, RETN | | | | | | | | | |
| RSAD2, IFI27, OAS1, IFIT1, IFI44L, ISG15, | 0.872 | 0.861 | 0.882 | 0.933 | 0.884 | 0.982 | 0.918 | 0.836 | 1 |
| HERC6, SLC1A2, OLAH, FAM20A | | | | | | | | | |
| IFI27, OAS1, IFIT1, IFI44L, ISG15, HERC6, | 0.871 | 0.861 | 0.882 | 0.935 | 0.887 | 0.982 | 0.918 | 0.835 | 1 |
| SLC1A2, OLAH, FAM20A, RETN | | | | | | | | | |
| RSAD2, IFI27, OAS1, IFI44L, SIGLEC1, | 0.871 | 0.862 | 0.881 | 0.927 | 0.874 | 0.981 | 0.950 | 0.895 | 1 |
| ISG15, HERC6, OLAH, RETN, MMP8 | | | | | | | | | |
| IFI27, OAS1, IFIT1, ISG15, SLC1A2, IL1R2, | 0.871 | 0.861 | 0.882 | 0.933 | 0.884 | 0.982 | 0.925 | 0.847 | 1 |
| OLAH, FAM20A, RETN, MMP8 | | | | | | | | | |
| RSAD2, IFI27, IFI44L, SIGLEC1, ISG15, | 0.871 | 0.861 | 0.882 | 0.938 | 0.892 | 0.983 | 0.948 | 0.877 | 1 |
| SLC1A2, IL1R2, FAM20A, RETN, MMP8 | | | | | | | | | |
| RSAD2, IFI27, IFI44L, SIGLEC1, ISG15, | 0.871 | 0.861 | 0.882 | 0.936 | 0.888 | 0.983 | 0.940 | 0.872 | 1 |
| SLC1A2, ILIR2, OLAH, FAM20A, RETN | | | | | | | | | |
| RSAD2, IFI27, OAS1, IFIT1, ISG15, HERC6, | 0.871 | 0.860 | 0.883 | 0.930 | 0.879 | 0.980 | 0.921 | 0.840 | 1 |
| SLC1A2, OLAH, FAM20A, MMP8 | | | | | | | | | |
| RSAD2, IFI27, OAS1, IFIT1, IFI44L, | 0.871 | 0.862 | 0.881 | 0.920 | 0.863 | 0.978 | 0.943 | 0.883 | 1 |
| SIGLEC1, ISG15, HERC6, RETN, MMP8 | | | | | | | | | |
| IFI27, OAS1, IFIT1, IFI44L, SIGLEC1, | 0.871 | 0.863 | 0.880 | 0.924 | 0.871 | 0.976 | 0.941 | 0.884 | 0.999 |
| ISG15, HERC6, IL1R2, OLAH, RETN | | | | | | | | | |
| RSAD2, IFI27, OAS1, IFI44L, SIGLEC1, | 0.871 | 0.86 | 0.882 | 0.937 | 0.892 | 0.983 | 0.948 | 0.877 | 1 |
| SLC1A2, ILIR2, FAM20A, RETN, MMP8 | | | | | | | | | |
| RSAD2, IFI27, OAS1, ISG15, HERC6, | 0.871 | 0.861 | 0.882 | 0.934 | 0.885 | 0.983 | 0.922 | 0.838 | 1 |
| SLC1A2, OLAH, FAM20A, RETN, MMP8 | | | | | | | | | |
| RSAD2, IFI27, OAS1, IFI44L, SIGLEC1, | 0.871 | 0.861 | 0.881 | 0.933 | 0.882 | 0.984 | 0.940 | 0.872 | 1 |
| ISG15, SLC1A2, OLAH, FAM20A, RETN | | | | | | | | | |
| RSAD2, IFI27, OAS1, SIGLEC1, HERC6, | 0.871 | 0.861 | 0.882 | 0.933 | 0.883 | 0.982 | 0.936 | 0.866 | 1 |
| SLC1A2, IL1R2, OLAH, FAM20A, RETN | | | | | | | | | |
| IFI27, OAS1, IFI44L, SIGLEC1, ISG15, | 0.871 | 0.86 | 0.882 | 0.935 | 0.891 | 0.980 | 0.941 | 0.869 | 1 |
| HERC6, SLC1A2, IL1R2, FAM20A, RETN | | | | | | | | | |
| OAS1, IFIT1, SIGLEC1, ISG15, HERC6, | 0.871 | 0.862 | 0.880 | 0.923 | 0.867 | 0.979 | 0.963 | 0.909 | 1 |
| IL1R2, OLAH, FAM20A, RETN, MMP8 | | | | | | | | | |
| RSAD2, IFI27, OAS1, IFI44L, SIGLEC1, | 0.871 | 0.862 | 0.880 | 0.924 | 0.871 | 0.978 | 0.942 | 0.883 | 1 |
| ISG15, HERC6, IL1R2, OLAH, RETN | | | | | | | | | |
| RSAD2, IFI27, OAS1, IFIT1, IFI44L, | 0.871 | 0.861 | 0.881 | 0.934 | 0.885 | 0.982 | 0.933 | 0.859 | 1 |
| SIGLEC1, HERC6, SLC1A2, FAM20A, | | | | | | | | | |
| RETN | | | | | | | | | |
| RSAD2, IFI27, OAS1, IFIT1, ISG15, HERC6, | 0.871 | 0.86 | 0.881 | 0.933 | 0.886 | 0.981 | 0.918 | 0.834 | 1 |
| SLC1A2, IL1R2, OLAH, FAM20A | | | | | | | | | |
| IFI27, OAS1, IFIT1, IFI44L, ISG15, SLC1A2, | 0.871 | 0.860 | 0.882 | 0.934 | 0.885 | 0.982 | 0.924 | 0.846 | 1 |
| IL1R2, OLAH, FAM20A, MMP8 | | | | | | | | | |
| RSAD2, IFI27, IFI44L, SIGLEC1, ISG15, | 0.871 | 0.862 | 0.880 | 0.928 | 0.876 | 0.980 | 0.952 | 0.902 | 1 |
| HERC6, IL1R2, OLAH, RETN, MMP8 | | | | | | | | | |
| RSAD2, IFI27, IFI44L, SIGLEC1, ISG15, | 0.871 | 0.859 | 0.883 | 0.938 | 0.890 | 0.986 | 0.945 | 0.879 | 1 |
| SLC1A2, OLAH, FAM20A, RETN, MMP8 | | | | | | | | | |
| RSAD2, IFI27, OAS1, IFI44L, SIGLEC1, | 0.871 | 0.860 | 0.882 | 0.933 | 0.88 | 0.985 | 0.950 | 0.889 | 1 |
| SLC1A2, OLAH, FAM20A, RETN, MMP8 | | | | | | | | | |
| RSAD2, IFI27, IFIT1, IFI44L, ISG15, | 0.871 | 0.860 | 0.881 | 0.932 | 0.880 | 0.983 | 0.936 | 0.858 | 1 |
| HERC6, IL1R2, FAM20A, RETN, MMP8 | | | | | | | | | |
| RSAD2, IFI27, IFIT1, IFI44L, SIGLEC1, | 0.871 | 0.860 | 0.882 | 0.931 | 0.879 | 0.983 | 0.942 | 0.878 | 1 |
| SLC1A2, IL1R2, OLAH, FAM20A, RETN | | | | | | | | | |
| RSAD2, OAS1, IFIT1, SIGLEC1, ISG15, | 0.871 | 0.86 | 0.881 | 0.921 | 0.866 | 0.976 | 0.962 | 0.906 | 1 |
| HERC6, IL1R2, OLAH, FAM20A, MMP8 | | | | | | | | | |
| RSAD2, IFI27, OAS1, IFIT1, SIGLEC1, | 0.871 | 0.861 | 0.880 | 0.932 | 0.883 | 0.981 | 0.934 | 0.860 | 1 |
| ISG15, HERC6, SLC1A2, FAM20A, RETN | | | | | | | | | |
| RSAD2, IFI27, OAS1, IFIT1, ISG15, HERC6, | 0.871 | 0.861 | 0.881 | 0.931 | 0.879 | 0.984 | 0.928 | 0.847 | 1 |
| IL1R2, FAM20A, RETN, MMP8 | | | | | | | | | |
| RSAD2, IFI27, IFIT1, SIGLEC1, ISG15, | 0.871 | 0.862 | 0.880 | 0.926 | 0.872 | 0.979 | 0.952 | 0.901 | 1 |
| HERC6, IL1R2, OLAH, RETN, MMP8 | | | | | | | | | |
| RSAD2, IFI27, OAS1, IFI44L, HERC6, | 0.871 | 0.859 | 0.882 | 0.935 | 0.885 | 0.984 | 0.924 | 0.844 | 1 |
| SLC1A2, OLAH, FAM20A, RETN, MMP8 | | | | | | | | | |
| RSAD2, IFI27, OAS1, IFIT1, IFI44L, ISG15, | 0.871 | 0.860 | 0.882 | 0.923 | 0.863 | 0.983 | 0.940 | 0.876 | 1 |
| OLAH, FAM20A, RETN, MMP8 | | | | | | | | | |
| IFI27, OAS1, IFIT1, IFI44L, SIGLEC1, | 0.871 | 0.861 | 0.881 | 0.934 | 0.887 | 0.980 | 0.943 | 0.875 | 1 |
| ISG15, SLC1A2, IL1R2, FAM20A, RETN | | | | | | | | | |
| IFI27, IFIT1, IFI44L, ISG15, HERC6, | 0.871 | 0.860 | 0.881 | 0.932 | 0.881 | 0.983 | 0.924 | 0.843 | 1 |
| SLC1A2, OLAH, FAM20A, RETN, MMP8 | | | | | | | | | |
| IFI27, IFIT1, IFI44L, SIGLEC1, ISG15, | 0.871 | 0.861 | 0.880 | 0.926 | 0.873 | 0.979 | 0.953 | 0.904 | 1 |
| HERC6, IL1R2, OLAH, RETN, MMP8 | | | | | | | | | |
| IFI27, OAS1, IFIT1, IFI44L, ISG15, HERC6, | 0.871 | 0.860 | 0.881 | 0.935 | 0.887 | 0.983 | 0.917 | 0.834 | 1 |
| SLC1A2, IL1R2, OLAH, FAM20A | | | | | | | | | |
| RSAD2, IFI27, OAS1, IFI44L, SIGLEC1, | 0.870 | 0.860 | 0.881 | 0.935 | 0.890 | 0.979 | 0.941 | 0.869 | 1 |
| ISG15, HERC6, SLC1A2, IL1R2, FAM20A | | | | | | | | | |
| RSAD2, IFI27, IFIT1, ISG15, SLC1A2, | 0.870 | 0.860 | 0.881 | 0.933 | 0.884 | 0.982 | 0.925 | 0.847 | 1 |
| IL1R2, OLAH, FAM20A, RETN, MMP8 | | | | | | | | | |
| RSAD2, IFI27, OAS1, IFIT1, IFI44L, | 0.870 | 0.861 | 0.880 | 0.927 | 0.874 | 0.979 | 0.953 | 0.901 | 1 |
| SIGLEC1, HERC6, IL1R2, RETN, MMP8 | | | | | | | | | |
| IFI27, OAS1, IFIT1, IFI44L, HERC6, | 0.870 | 0.860 | 0.881 | 0.934 | 0.885 | 0.982 | 0.923 | 0.842 | 1 |
| SLC1A2, ILIR2, OLAH, FAM20A, RETN | | | | | | | | | |
| IFI27, IFIT1, ISG15, HERC6, SLC1A2, | 0.870 | 0.860 | 0.881 | 0.935 | 0.887 | 0.982 | 0.925 | 0.846 | 1 |
| IL1R2, OLAH, FAM20A, RETN, MMP8 | | | | | | | | | |
| RSAD2, IFI27, OAS1, IFIT1, IFI44L, | 0.870 | 0.861 | 0.880 | 0.923 | 0.867 | 0.979 | 0.949 | 0.895 | 1 |
| HERC6, IL1R2, OLAH, RETN, MMP8 | | | | | | | | | |
| RSAD2, IFI27, OAS1, IFIT1, IFI44L, ISG15, | 0.870 | 0.861 | 0.880 | 0.924 | 0.867 | 0.981 | 0.945 | 0.887 | 1 |
| HERC6, OLAH, RETN, MMP8 | | | | | | | | | |
| RSAD2, IFI27, OAS1, IFIT1, IFI44L, | 0.870 | 0.860 | 0.880 | 0.934 | 0.886 | 0.982 | 0.922 | 0.839 | 1 |
| HERC6, SLC1A2, OLAH, FAM20A, RETN | | | | | | | | | |
| RSAD2, IFI27, OAS1, IFIT1, IFI44L, ISG15, | 0.870 | 0.860 | 0.881 | 0.928 | 0.877 | 0.979 | 0.930 | 0.849 | 1 |
| HERC6, IL1R2, FAM20A, MMP8 | | | | | | | | | |
| RSAD2, IFI27, IFIT1, IFI44L, SIGLEC1, | 0.870 | 0.861 | 0.879 | 0.925 | 0.871 | 0.980 | 0.951 | 0.899 | 1 |
| ISG15, HERC6, OLAH, RETN, MMP8 | | | | | | | | | |
| RSAD2, IFI27, OAS1, IFI44L, ISG15, | 0.870 | 0.859 | 0.882 | 0.932 | 0.880 | 0.984 | 0.929 | 0.846 | 1 |
| HERC6, IL1R2, FAM20A, RETN, MMP8 | | | | | | | | | |
| OAS1, IFIT1, IFI44L, SIGLEC1, HERC6, | 0.870 | 0.861 | 0.880 | 0.926 | 0.871 | 0.980 | 0.964 | 0.911 | 1 |
| IL1R2, OLAH, FAM20A, RETN, MMP8 | | | | | | | | | |
| RSAD2, IFI27, IFI44L, ISG15, SLC1A2, | 0.870 | 0.859 | 0.881 | 0.935 | 0.887 | 0.984 | 0.924 | 0.845 | 1 |
| IL1R2, OLAH, FAM20A, RETN, MMP8 | | | | | | | | | |
| IFI27, OAS1, IFIT1, SIGLEC1, HERC6, | 0.870 | 0.860 | 0.880 | 0.932 | 0.884 | 0.980 | 0.949 | 0.890 | 1 |
| SLC1A2, IL1R2, OLAH, RETN, MMP8 | | | | | | | | | |
| RSAD2, IFI27, OAS1, IFIT1, ISG15, HERC6, | 0.870 | 0.860 | 0.880 | 0.924 | 0.868 | 0.980 | 0.945 | 0.885 | 1 |
| IL1R2, OLAH, RETN, MMP8 | | | | | | | | | |
| IFI27, IFIT1, IFI44L, SIGLEC1, ISG15, | 0.870 | 0.860 | 0.881 | 0.930 | 0.877 | 0.983 | 0.951 | 0.879 | 1 |
| HERC6, SLC1A2, FAM20A, RETN, MMP8 | | | | | | | | | |
| RSAD2, IFI27, OAS1, IFIT1, IFI44L, | 0.870 | 0.859 | 0.881 | 0.923 | 0.864 | 0.982 | 0.947 | 0.877 | 1 |
| SIGLEC1, ISG15, FAM20A, RETN, MMP8 | | | | | | | | | |
| RSAD2, IFI27, OAS1, IFIT1, IFI44L, | 0.870 | 0.862 | 0.879 | 0.924 | 0.871 | 0.977 | 0.954 | 0.904 | 1 |
| SIGLEC1, ISG15, HERC6, IL1R2, RETN | | | | | | | | | |
| RSAD2, IFI27, OAS1, IFIT1, IFI44L, ISG15, | 0.870 | 0.860 | 0.880 | 0.931 | 0.880 | 0.982 | 0.927 | 0.844 | 1 |
| HERC6, IL1R2, FAM20A, RETN | | | | | | | | | |
| IFI27, OAS1, IFIT1, IFI44L, ISG15, SLC1A2, | 0.870 | 0.859 | 0.881 | 0.931 | 0.878 | 0.984 | 0.924 | 0.846 | 1 |
| OLAH, FAM20A, RETN, MMP8 | | | | | | | | | |
| RSAD2, IFI27, OAS1, IFIT1, IFI44L, | 0.870 | 0.861 | 0.879 | 0.927 | 0.878 | 0.976 | 0.947 | 0.896 | 0.998 |
| SIGLEC1, ISG15, HERC6, IL1R2, OLAH | | | | | | | | | |
| OAS1, IFIT1, IFI44L, SIGLEC1, ISG15, | 0.870 | 0.860 | 0.879 | 0.923 | 0.868 | 0.979 | 0.964 | 0.909 | 1 |
| HERC6, IL1R2, FAM20A, RETN, MMP8 | | | | | | | | | |
| IFI27, OAS1, IFI44L, ISG15, HERC6, | 0.870 | 0.859 | 0.881 | 0.935 | 0.887 | 0.983 | 0.917 | 0.833 | 1 |
| SLC1A2, IL1R2, OLAH, FAM20A, RETN | | | | | | | | | |
| RSAD2, IFI27, IFIT1, IFI44L, SIGLEC1, | 0.870 | 0.859 | 0.881 | 0.935 | 0.888 | 0.981 | 0.943 | 0.873 | 1 |
| HERC6, SLC1A2, IL1R2, FAM20A, RETN | | | | | | | | | |
| OAS1, IFIT1, IFI44L, SIGLEC1, ISG15, | 0.870 | 0.859 | 0.880 | 0.923 | 0.869 | 0.977 | 0.963 | 0.909 | 1 |
| HERC6, ILIR2, OLAH, FAM20A, MMP8 | | | | | | | | | |
| RSAD2, IFI27, IFI44L, ISG15, HERC6, | 0.870 | 0.858 | 0.881 | 0.936 | 0.888 | 0.985 | 0.923 | 0.843 | 1 |
| SLC1A2, OLAH, FAM20A, RETN, MMP8 | | | | | | | | | |
| RSAD2, IFI27, OAS1, IFI44L, ISG15, | 0.870 | 0.859 | 0.880 | 0.934 | 0.885 | 0.983 | 0.918 | 0.834 | 1 |
| HERC6, SLC1A2, OLAH, FAM20A, RETN | | | | | | | | | |
| IFI27, OAS1, IFIT1, IFI44L, SIGLEC1, | 0.870 | 0.860 | 0.879 | 0.926 | 0.875 | 0.978 | 0.954 | 0.906 | 1 |
| ISG15, HERC6, IL1R2, RETN, MMP8 | | | | | | | | | |
| RSAD2, IFI27, OAS1, IFI44L, ISG15, | 0.870 | 0.860 | 0.879 | 0.924 | 0.868 | 0.980 | 0.943 | 0.885 | 1 |
| HERC6, IL1R2, OLAH, RETN, MMP8 | | | | | | | | | |
| RSAD2, IFI27, OAS1, IFIT1, IFI44L, | 0.870 | 0.860 | 0.879 | 0.934 | 0.888 | 0.980 | 0.942 | 0.873 | 1 |
| SIGLEC1, ISG15, SLC1A2, IL1R2, FAM20A | | | | | | | | | |
| RSAD2, IFI27, OAS1, IFI44L, SIGLEC1, | 0.870 | 0.861 | 0.879 | 0.926 | 0.876 | 0.976 | 0.947 | 0.896 | 0.997 |
| ISG15, HERC6, IL1R2, OLAH, MMP8 | | | | | | | | | |
| RSAD2, IFI27, OAS1, IFIT1, SLC1A2, | 0.869 | 0.859 | 0.880 | 0.932 | 0.881 | 0.983 | 0.924 | 0.846 | 1 |
| IL1R2, OLAH, FAM20A, RETN, MMP8 | | | | | | | | | |
| RSAD2, IFI27, OAS1, ISG15, HERC6, | 0.869 | 0.859 | 0.880 | 0.935 | 0.888 | 0.982 | 0.918 | 0.833 | 1 |
| SLC1A2, ILIR2, OLAH, FAM20A, RETN | | | | | | | | | |
| IFI27, OAS1, IFIT1, IFI44L, ISG15, HERC6, | 0.869 | 0.860 | 0.879 | 0.924 | 0.868 | 0.979 | 0.949 | 0.894 | 1 |
| IL1R2, OLAH, RETN, MMP8 | | | | | | | | | |
| RSAD2, IFI27, OAS1, IFIT1, IFI44L, | 0.869 | 0.860 | 0.878 | 0.926 | 0.878 | 0.975 | 0.949 | 0.900 | 0.998 |
| SIGLEC1, HERC6, IL1R2, OLAH, MMP8 | | | | | | | | | |
| RSAD2, IFI27, IFIT1, IFI44L, SIGLEC1, | 0.869 | 0.860 | 0.879 | 0.932 | 0.882 | 0.982 | 0.939 | 0.866 | 1 |
| ISG15, HERC6, SLC1A2, FAM20A, RETN | | | | | | | | | |
| IFIT1, IFI44L, SIGLEC1, ISG15, HERC6, | 0.869 | 0.859 | 0.879 | 0.924 | 0.869 | 0.980 | 0.963 | 0.907 | 1 |
| IL1R2, OLAH, FAM20A, RETN, MMP8 | | | | | | | | | |
| RSAD2, IFI27, IFIT1, IFI44L, SIGLEC1, | 0.869 | 0.861 | 0.878 | 0.926 | 0.874 | 0.979 | 0.946 | 0.893 | 0.998 |
| ISG15, HERC6, IL1R2, OLAH, RETN | | | | | | | | | |
| RSAD2, IFI27, OAS1, IFIT1, SIGLEC1, | 0.869 | 0.860 | 0.879 | 0.933 | 0.887 | 0.980 | 0.942 | 0.873 | 1 |
| ISG15, SLC1A2, IL1R2, FAM20A, RETN | | | | | | | | | |
| IFI27, OAS1, IFIT1, ISG15, HERC6, | 0.869 | 0.859 | 0.880 | 0.934 | 0.886 | 0.982 | 0.916 | 0.830 | 1 |
| SLC1A2, IL1R2, OLAH, FAM20A, RETN | | | | | | | | | |
| RSAD2, OAS1, IFIT1, IFI44L, SIGLEC1, | 0.869 | 0.859 | 0.879 | 0.922 | 0.867 | 0.977 | 0.963 | 0.907 | 1 |
| ISG15, HERC6, IL1R2, FAM20A, MMP8 | | | | | | | | | |
| RSAD2, IFI27, IFI44L, SIGLEC1, HERC6, | 0.869 | 0.858 | 0.880 | 0.934 | 0.885 | 0.983 | 0.943 | 0.876 | 1 |
| SLC1A2, IL1R2, OLAH, FAM20A, RETN | | | | | | | | | |
| IFI27, OAS1, IFIT1, IFI44L, SIGLEC1, | 0.869 | 0.860 | 0.878 | 0.928 | 0.879 | 0.976 | 0.941 | 0.888 | 0.995 |
| ISG15, HERC6, IL1R2, OLAH, MMP8 | | | | | | | | | |
| RSAD2, OAS1, IFIT1, SIGLEC1, HERC6, | 0.869 | 0.860 | 0.879 | 0.924 | 0.869 | 0.980 | 0.962 | 0.907 | 1 |
| IL1R2, OLAH, FAM20A, RETN, MMP8 | | | | | | | | | |
| RSAD2, OAS1, IFIT1, SIGLEC1, ISG15, | 0.869 | 0.860 | 0.878 | 0.923 | 0.867 | 0.979 | 0.961 | 0.903 | 1 |
| HERC6, IL1R2, FAM20A, RETN, MMP8 | | | | | | | | | |
| IFI27, OAS1, IFI44L, ISG15, SLC1A2, | 0.869 | 0.858 | 0.880 | 0.935 | 0.887 | 0.984 | 0.924 | 0.845 | 1 |
| ILIR2, OLAH, FAM20A, RETN, MMP8 | | | | | | | | | |
| RSAD2, IFI27, OAS1, IFIT1, IFI44L, | 0.869 | 0.859 | 0.879 | 0.923 | 0.866 | 0.981 | 0.937 | 0.865 | 1 |
| SIGLEC1, ISG15, HERC6, FAM20A, MMP8 | | | | | | | | | |
| RSAD2, IFI27, IFIT1, SIGLEC1, ISG15, | 0.869 | 0.859 | 0.879 | 0.934 | 0.887 | 0.981 | 0.945 | 0.873 | 1 |
| HERC6, SLC1A2, IL1R2, FAM20A, RETN | | | | | | | | | |
| RSAD2, IFI27, OAS1, IFIT1, IFI44L, ISG15, | 0.869 | 0.858 | 0.880 | 0.925 | 0.868 | 0.983 | 0.932 | 0.853 | 1 |
| HERC6, FAM20A, RETN, MMP8 | | | | | | | | | |
| RSAD2, IFI27, OAS1, SIGLEC1, ISG15, | 0.869 | 0.858 | 0.880 | 0.930 | 0.876 | 0.984 | 0.941 | 0.867 | 1 |
| HERC6, SLC1A2, FAM20A, RETN, MMP8 | | | | | | | | | |
| RSAD2, IFI27, OAS1, IFIT1, IFI44L, ISG15, | 0.869 | 0.858 | 0.880 | 0.924 | 0.867 | 0.981 | 0.929 | 0.859 | 1.000 |
| ILIR2, OLAH, FAM20A, RETN | | | | | | | | | |
| RSAD2, IFI27, IFIT1, IFI44L, ISG15, | 0.869 | 0.859 | 0.879 | 0.935 | 0.888 | 0.983 | 0.917 | 0.836 | 0.999 |
| HERC6, SLC1A2, IL1R2, OLAH, FAM20A | | | | | | | | | |
| RSAD2, IFI27, IFIT1, IFI44L, SLC1A2, | 0.869 | 0.859 | 0.879 | 0.934 | 0.885 | 0.984 | 0.926 | 0.850 | 1 |
| IL1R2, OLAH, FAM20A, RETN, MMP8 | | | | | | | | | |
| IFI27, OAS1, IFIT1, SIGLEC1, ISG15, | 0.869 | 0.859 | 0.879 | 0.931 | 0.882 | 0.981 | 0.939 | 0.876 | 1 |
| HERC6, SLC1A2, IL1R2, OLAH, RETN | | | | | | | | | |
| RSAD2, IFI27, OAS1, IFIT1, SIGLEC1, | 0.869 | 0.860 | 0.878 | 0.925 | 0.872 | 0.978 | 0.953 | 0.900 | 1 |
| ISG15, HERC6, IL1R2, RETN, MMP8 | | | | | | | | | |
| RSAD2, IFI27, OAS1, IFIT1, ISG15, | 0.869 | 0.858 | 0.880 | 0.929 | 0.876 | 0.982 | 0.927 | 0.852 | 1 |
| SLC1A2, OLAH, FAM20A, RETN, MMP8 | | | | | | | | | |
| RSAD2, IFI27, IFIT1, IFI44L, HERC6, | 0.869 | 0.859 | 0.879 | 0.934 | 0.885 | 0.983 | 0.920 | 0.838 | 1 |
| SLC1A2, IL1R2, OLAH, FAM20A, RETN | | | | | | | | | |
| IFI27, OAS1, SIGLEC1, ISG15, HERC6, | 0.869 | 0.859 | 0.879 | 0.932 | 0.883 | 0.981 | 0.947 | 0.886 | 1 |
| SLC1A2, IL1R2, OLAH, RETN, MMP8 | | | | | | | | | |
| RSAD2, IFIT1, IFI44L, SIGLEC1, ISG15, | 0.869 | 0.858 | 0.879 | 0.923 | 0.869 | 0.977 | 0.963 | 0.909 | 1 |
| HERC6, IL1R2, OLAH, FAM20A, MMP8 | | | | | | | | | |
| RSAD2, IFI27, OAS1, IFIT1, IFI44L, ISG15, | 0.869 | 0.860 | 0.878 | 0.923 | 0.867 | 0.978 | 0.939 | 0.879 | 0.999 |
| HERC6, IL1R2, OLAH, RETN | | | | | | | | | |
| RSAD2, IFI27, OAS1, IFI44L, SIGLEC1, | 0.869 | 0.858 | 0.880 | 0.930 | 0.878 | 0.983 | 0.943 | 0.878 | 1 |
| SLC1A2, ILIR2, OLAH, FAM20A, RETN | | | | | | | | | |
| RSAD2, IFI27, IFIT1, IFI44L, ISG15, | 0.869 | 0.858 | 0.880 | 0.931 | 0.879 | 0.984 | 0.925 | 0.848 | 1 |
| SLC1A2, OLAH, FAM20A, RETN, MMP8 | | | | | | | | | |
| RSAD2, IFI27, OAS1, IFI44L, SIGLEC1, | 0.869 | 0.858 | 0.879 | 0.935 | 0.890 | 0.980 | 0.939 | 0.865 | 1 |
| HERC6, SLC1A2, IL1R2, FAM20A, RETN | | | | | | | | | |
| IFI27, OAS1, IFIT1, IFI44L, SIGLEC1, | 0.869 | 0.859 | 0.879 | 0.932 | 0.883 | 0.980 | 0.947 | 0.887 | 1 |
| HERC6, SLC1A2, OLAH, RETN, MMP8 | | | | | | | | | |
| IFI27, OAS1, IFIT1, IFI44L, SLC1A2, IL1R2, | 0.869 | 0.858 | 0.879 | 0.934 | 0.885 | 0.984 | 0.925 | 0.848 | 1 |
| OLAH, FAM20A, RETN, MMP8 | | | | | | | | | |
| RSAD2, IFI27, OAS1, IFIT1, HERC6, | 0.869 | 0.859 | 0.879 | 0.933 | 0.884 | 0.981 | 0.917 | 0.831 | 1 |
| SLC1A2, ILIR2, OLAH, FAM20A, RETN | | | | | | | | | |
| RSAD2, IFI27, IFIT1, IFI44L, ISG15, | 0.869 | 0.858 | 0.879 | 0.936 | 0.888 | 0.984 | 0.918 | 0.837 | 1 |
| HERC6, SLC1A2, OLAH, FAM20A, RETN | | | | | | | | | |
| RSAD2, IFI27, OAS1, IFIT1, SIGLEC1, | 0.869 | 0.859 | 0.878 | 0.931 | 0.881 | 0.982 | 0.946 | 0.885 | 1 |
| HERC6, SLC1A2, IL1R2, OLAH, RETN | | | | | | | | | |
| IFI27, OAS1, IFIT1, SIGLEC1, ISG15, | 0.869 | 0.858 | 0.879 | 0.931 | 0.882 | 0.980 | 0.948 | 0.888 | 1 |
| HERC6, SLC1A2, OLAH, RETN, MMP8 | | | | | | | | | |
| IFI27, OAS1, IFIT1, IFI44L, HERC6, | 0.868 | 0.858 | 0.878 | 0.933 | 0.884 | 0.982 | 0.927 | 0.843 | 1 |
| SLC1A2, IL1R2, FAM20A, RETN, MMP8 | | | | | | | | | |
| RSAD2, IFI27, OAS1, IFIT1, SIGLEC1, | 0.868 | 0.858 | 0.879 | 0.932 | 0.883 | 0.982 | 0.950 | 0.892 | 1 |
| HERC6, SLC1A2, OLAH, RETN, MMP8 | | | | | | | | | |
| RSAD2, IFI27, OAS1, IFIT1, IFI44L, | 0.868 | 0.859 | 0.877 | 0.931 | 0.882 | 0.980 | 0.945 | 0.882 | 1 |
| SIGLEC1, HERC6, SLC1A2, OLAH, RETN | | | | | | | | | |
| RSAD2, IFI27, OAS1, IFIT1, IFI44L, | 0.868 | 0.859 | 0.877 | 0.925 | 0.873 | 0.977 | 0.941 | 0.886 | 0.997 |
| SIGLEC1, ISG15, HERC6, OLAH, MMP8 | | | | | | | | | |
| RSAD2, IFIT1, SIGLEC1, ISG15, HERC6, | 0.868 | 0.859 | 0.878 | 0.923 | 0.867 | 0.979 | 0.962 | 0.906 | 1 |
| IL1R2, OLAH, FAM20A, RETN, MMP8 | | | | | | | | | |
| RSAD2, IFI27, IFI44L, ISG15, HERC6, | 0.868 | 0.858 | 0.879 | 0.935 | 0.888 | 0.983 | 0.916 | 0.832 | 1 |
| SLC1A2, IL1R2, OLAH, FAM20A, RETN | | | | | | | | | |
| RSAD2, IFI27, OAS1, ISG15, SLC1A2, | 0.868 | 0.858 | 0.879 | 0.935 | 0.887 | 0.983 | 0.922 | 0.841 | 1 |
| IL1R2, OLAH, FAM20A, RETN, MMP8 | | | | | | | | | |
| RSAD2, IFI27, OAS1, SIGLEC1, ISG15, | 0.868 | 0.857 | 0.879 | 0.934 | 0.888 | 0.980 | 0.941 | 0.870 | 1 |
| HERC6, SLC1A2, IL1R2, FAM20A, RETN | | | | | | | | | |
| RSAD2, IFI27, OAS1, IFI44L, SIGLEC1, | 0.868 | 0.859 | 0.877 | 0.926 | 0.874 | 0.979 | 0.955 | 0.905 | 1 |
| ISG15, HERC6, IL1R2, RETN, MMP8 | | | | | | | | | |
| RSAD2, IFI27, IFIT1, SIGLEC1, HERC6, | 0.868 | 0.858 | 0.878 | 0.932 | 0.883 | 0.980 | 0.951 | 0.899 | 1 |
| SLC1A2, IL1R2, OLAH, RETN, MMP8 | | | | | | | | | |
| RSAD2, IFIT1, IFI44L, SIGLEC1, HERC6, | 0.868 | 0.858 | 0.878 | 0.929 | 0.883 | 0.976 | 0.955 | 0.891 | 1 |
| SLC1A2, IL1R2, OLAH, FAM20A, MMP8 | | | | | | | | | |
| RSAD2, OAS1, IFIT1, IFI44L, SIGLEC1, | 0.868 | 0.858 | 0.878 | 0.924 | 0.869 | 0.979 | 0.963 | 0.908 | 1 |
| HERC6, IL1R2, FAM20A, RETN, MMP8 | | | | | | | | | |
| RSAD2, IFI27, OAS1, IFIT1, ISG15, HERC6, | 0.868 | 0.857 | 0.878 | 0.934 | 0.885 | 0.982 | 0.916 | 0.829 | 1 |
| SLC1A2, OLAH, FAM20A, RETN | | | | | | | | | |
| IFI27, OAS1, IFIT1, IFI44L, SIGLEC1, | 0.868 | 0.858 | 0.877 | 0.925 | 0.871 | 0.979 | 0.954 | 0.901 | 1 |
| ISG15, IL1R2, OLAH, RETN, MMP8 | | | | | | | | | |
| RSAD2, IFI27, IFIT1, ISG15, HERC6, | 0.868 | 0.858 | 0.878 | 0.930 | 0.880 | 0.98 | 0.915 | 0.830 | 1 |
| SLC1A2, IL1R2, OLAH, FAM20A, RETN | | | | | | | | | |
| RSAD2, IFI27, IFIT1, IFI44L, ISG15, | 0.868 | 0.858 | 0.877 | 0.924 | 0.869 | 0.979 | 0.946 | 0.891 | 1 |
| HERC6, IL1R2, OLAH, RETN, MMP8 | | | | | | | | | |
| IFIT1, IFI44L, SIGLEC1, ISG15, HERC6, | 0.868 | 0.858 | 0.877 | 0.928 | 0.880 | 0.975 | 0.960 | 0.897 | 1 |
| SLC1A2, IL1R2, OLAH, FAM20A, MMP8 | | | | | | | | | |
| RSAD2, IFI27, IFIT1, IFI44L, SIGLEC1, | 0.868 | 0.858 | 0.877 | 0.934 | 0.888 | 0.980 | 0.943 | 0.874 | 1 |
| ISG15, SLC1A2, IL1R2, FAM20A, RETN | | | | | | | | | |
| RSAD2, IFI27, OAS1, IFI44L, HERC6, | 0.868 | 0.857 | 0.878 | 0.932 | 0.883 | 0.982 | 0.918 | 0.835 | 1 |
| SLC1A2, IL1R2, OLAH, FAM20A, RETN | | | | | | | | | |
| IFI27, IFIT1, IFI44L, ISG15, HERC6, | 0.868 | 0.857 | 0.878 | 0.933 | 0.883 | 0.982 | 0.916 | 0.833 | 1.000 |
| SLC1A2, ILIR2, OLAH, FAM20A, RETN | | | | | | | | | |
| RSAD2, IFI27, OAS1, SIGLEC1, HERC6, | 0.867 | 0.857 | 0.878 | 0.931 | 0.882 | 0.980 | 0.948 | 0.888 | 1 |
| SLC1A2, IL1R2, OLAH, RETN, MMP8 | | | | | | | | | |
| RSAD2, IFI27, IFIT1, IFI44L, SIGLEC1, | 0.867 | 0.859 | 0.876 | 0.928 | 0.879 | 0.977 | 0.940 | 0.886 | 0.995 |
| ISG15, HERC6, IL1R2, OLAH, MMP8 | | | | | | | | | |
| IFI27, OAS1, IFIT1, IFI44L, ISG15, SLC1A2, | 0.867 | 0.857 | 0.878 | 0.930 | 0.877 | 0.982 | 0.921 | 0.840 | 1 |
| ILIR2, OLAH, FAM20A, RETN | | | | | | | | | |
| IFI27, OAS1, IFI44L, SIGLEC1, ISG15, | 0.867 | 0.856 | 0.879 | 0.932 | 0.879 | 0.984 | 0.942 | 0.869 | 1 |
| HERC6, SLC1A2, FAM20A, RETN, MMP8 | | | | | | | | | |
| OAS1, IFIT1, IFI44L, SIGLEC1, HERC6, | 0.867 | 0.857 | 0.878 | 0.929 | 0.882 | 0.976 | 0.954 | 0.892 | 1 |
| SLC1A2, IL1R2, OLAH, FAM20A, MMP8 | | | | | | | | | |
| RSAD2, IFI27, OAS1, IFIT1, SIGLEC1, | 0.867 | 0.858 | 0.877 | 0.924 | 0.875 | 0.974 | 0.943 | 0.891 | 0.996 |
| ISG15, HERC6, IL1R2, OLAH, MMP8 | | | | | | | | | |
| IFI27, OAS1, IFI44L, SIGLEC1, HERC6, | 0.867 | 0.857 | 0.877 | 0.931 | 0.882 | 0.980 | 0.949 | 0.891 | 1 |
| SLC1A2, IL1R2, OLAH, RETN, MMP8 | | | | | | | | | |
| RSAD2, IFI27, OAS1, IFIT1, IFI44L, ISG15, | 0.867 | 0.857 | 0.877 | 0.931 | 0.879 | 0.982 | 0.921 | 0.839 | 1 |
| SLC1A2, IL1R2, OLAH, FAM20A | | | | | | | | | |
| RSAD2, IFI27, OAS1, IFI44L, SIGLEC1, | 0.867 | 0.857 | 0.877 | 0.931 | 0.882 | 0.981 | 0.949 | 0.890 | 1 |
| HERC6, SLC1A2, OLAH, RETN, MMP8 | | | | | | | | | |
| RSAD2, IFI27, OAS1, IFI44L, SLC1A2, | 0.867 | 0.856 | 0.878 | 0.937 | 0.889 | 0.985 | 0.927 | 0.851 | 1 |
| IL1R2, OLAH, FAM20A, RETN, MMP8 | | | | | | | | | |
| RSAD2, IFI27, OAS1, IFIT1, IFI44L, | 0.867 | 0.858 | 0.877 | 0.925 | 0.872 | 0.979 | 0.957 | 0.903 | 1 |
| SIGLEC1, IL1R2, OLAH, RETN, MMP8 | | | | | | | | | |
| RSAD2, IFI27, OAS1, IFIT1, IFI44L, | 0.867 | 0.857 | 0.878 | 0.934 | 0.886 | 0.981 | 0.943 | 0.877 | 1 |
| SIGLEC1, SLC1A2, ILIR2, FAM20A, RETN | | | | | | | | | |
| RSAD2, OAS1, IFIT1, SIGLEC1, HERC6, | 0.867 | 0.857 | 0.877 | 0.928 | 0.882 | 0.975 | 0.962 | 0.904 | 1 |
| SLC1A2, IL1R2, OLAH, FAM20A, MMP8 | | | | | | | | | |
| RSAD2, IFI27, OAS1, IFI44L, SIGLEC1, | 0.867 | 0.857 | 0.878 | 0.934 | 0.888 | 0.979 | 0.941 | 0.869 | 1 |
| ISG15, SLC1A2, IL1R2, FAM20A, RETN | | | | | | | | | |
| RSAD2, IFI27, OAS1, SIGLEC1, ISG15, | 0.867 | 0.858 | 0.877 | 0.929 | 0.879 | 0.980 | 0.946 | 0.885 | 1 |
| HERC6, SLC1A2, IL1R2, OLAH, RETN | | | | | | | | | |
| IFI27, OAS1, IFIT1, IFI44L, SIGLEC1, | 0.867 | 0.857 | 0.877 | 0.927 | 0.876 | 0.978 | 0.935 | 0.859 | 1 |
| ISG15, HERC6, SLC1A2, FAM20A, MMP8 | | | | | | | | | |
| RSAD2, IFI27, IFIT1, IFI44L, ISG15, | 0.867 | 0.857 | 0.877 | 0.931 | 0.880 | 0.982 | 0.917 | 0.836 | 0.999 |
| SLC1A2, IL1R2, OLAH, FAM20A, RETN | | | | | | | | | |
| IFI27, OAS1, IFIT1, IFI44L, SIGLEC1, | 0.867 | 0.856 | 0.878 | 0.927 | 0.871 | 0.982 | 0.941 | 0.867 | 1 |
| ISG15, SLC1A2, FAM20A, RETN, MMP8 | | | | | | | | | |
| RSAD2, IFI27, IFI44L, SIGLEC1, HERC6, | 0.867 | 0.858 | 0.876 | 0.930 | 0.880 | 0.979 | 0.952 | 0.901 | 1 |
| SLC1A2, IL1R2, OLAH, RETN, MMP8 | | | | | | | | | |
| RSAD2, IFI27, OAS1, IFIT1, IFI44L, | 0.867 | 0.856 | 0.878 | 0.929 | 0.874 | 0.983 | 0.928 | 0.853 | 1 |
| SLC1A2, OLAH, FAM20A, RETN, MMP8 | | | | | | | | | |
| RSAD2, OAS1, IFIT1, IFI44L, SIGLEC1, | 0.867 | 0.857 | 0.877 | 0.920 | 0.863 | 0.976 | 0.966 | 0.913 | 1 |
| ISG15, IL1R2, OLAH, FAM20A, MMP8 | | | | | | | | | |
| RSAD2, IFI27, IFIT1, IFI44L, SIGLEC1, | 0.867 | 0.858 | 0.876 | 0.924 | 0.870 | 0.979 | 0.954 | 0.902 | 1 |
| ISG15, IL1R2, OLAH, RETN, MMP8 | | | | | | | | | |
| RSAD2, IFI27, OAS1, IFI44L, SIGLEC1, | 0.867 | 0.857 | 0.876 | 0.931 | 0.882 | 0.980 | 0.945 | 0.883 | 1 |
| HERC6, SLC1A2, IL1R2, OLAH, RETN | | | | | | | | | |
| RSAD2, IFI27, OAS1, IFI44L, ISG15, | 0.867 | 0.855 | 0.879 | 0.930 | 0.877 | 0.983 | 0.927 | 0.851 | 1 |
| SLC1A2, OLAH, FAM20A, RETN, MMP8 | | | | | | | | | |
| RSAD2, OAS1, IFIT1, IFI44L, SIGLEC1, | 0.867 | 0.856 | 0.878 | 0.928 | 0.879 | 0.977 | 0.936 | 0.864 | 1 |
| HERC6, SLC1A2, OLAH, FAM20A, MMP8 | | | | | | | | | |
| IFI27, OAS1, IFIT1, IFI44L, SIGLEC1, | 0.867 | 0.857 | 0.876 | 0.929 | 0.880 | 0.979 | 0.939 | 0.876 | 1 |
| HERC6, SLC1A2, IL1R2, OLAH, RETN | | | | | | | | | |
| RSAD2, IFIT1, IFI44L, SIGLEC1, ISG15, | 0.867 | 0.857 | 0.876 | 0.924 | 0.868 | 0.980 | 0.961 | 0.903 | 1 |
| HERC6, IL1R2, FAM20A, RETN, MMP8 | | | | | | | | | |
| RSAD2, IFI27, OAS1, IFIT1, ISG15, | 0.867 | 0.857 | 0.877 | 0.930 | 0.880 | 0.981 | 0.921 | 0.838 | 1 |
| SLC1A2, IL1R2, OLAH, FAM20A, RETN | | | | | | | | | |
| IFI27, OAS1, IFIT1, IFI44L, SIGLEC1, | 0.867 | 0.857 | 0.876 | 0.930 | 0.882 | 0.979 | 0.937 | 0.873 | 1 |
| ISG15, HERC6, SLC1A2, OLAH, RETN | | | | | | | | | |
| OAS1, IFIT1, IFI44L, SIGLEC1, ISG15, | 0.867 | 0.856 | 0.877 | 0.922 | 0.864 | 0.979 | 0.952 | 0.896 | 1 |
| HERC6, OLAH, FAM20A, RETN, MMP8 | | | | | | | | | |
| RSAD2, OAS1, IFIT1, IFI44L, SIGLEC1, | 0.867 | 0.857 | 0.876 | 0.919 | 0.861 | 0.976 | 0.953 | 0.898 | 1 |
| ISG15, HERC6, IL1R2, OLAH, FAM20A | | | | | | | | | |
| RSAD2, IFI27, OAS1, IFI44L, SIGLEC1, | 0.867 | 0.856 | 0.878 | 0.930 | 0.877 | 0.984 | 0.943 | 0.871 | 1 |
| HERC6, SLC1A2, FAM20A, RETN, MMP8 | | | | | | | | | |
| RSAD2, IFI27, OAS1, IFI44L, ISG15, | 0.867 | 0.856 | 0.877 | 0.931 | 0.880 | 0.982 | 0.920 | 0.837 | 1 |
| SLC1A2, IL1R2, OLAH, FAM20A, RETN | | | | | | | | | |
| IFI27, OAS1, IFI44L, SIGLEC1, ISG15, | 0.867 | 0.856 | 0.877 | 0.932 | 0.883 | 0.981 | 0.946 | 0.884 | 1 |
| HERC6, SLC1A2, OLAH, RETN, MMP8 | | | | | | | | | |
| RSAD2, IFI27, OAS1, IFIT1, SIGLEC1, | 0.867 | 0.857 | 0.876 | 0.932 | 0.883 | 0.981 | 0.946 | 0.885 | 1 |
| ISG15, HERC6, SLC1A2, OLAH, RETN | | | | | | | | | |
| RSAD2, IFI27, OAS1, SIGLEC1, ISG15, | 0.866 | 0.856 | 0.877 | 0.931 | 0.881 | 0.980 | 0.948 | 0.888 | 1 |
| HERC6, SLC1A2, OLAH, RETN, MMP8 | | | | | | | | | |
| RSAD2, IFI27, OAS1, IFIT1, SIGLEC1, | 0.866 | 0.857 | 0.876 | 0.929 | 0.879 | 0.978 | 0.942 | 0.884 | 1 |
| ISG15, HERC6, SLC1A2, IL1R2, OLAH | | | | | | | | | |
| RSAD2, IFI27, OAS1, IFIT1, IFI44L, | 0.866 | 0.857 | 0.876 | 0.919 | 0.861 | 0.978 | 0.953 | 0.898 | 1 |
| SIGLEC1, ISG15, OLAH, RETN, MMP8 | | | | | | | | | |
| RSAD2, IFI27, IFI44L, SIGLEC1, ISG15, | 0.866 | 0.856 | 0.877 | 0.933 | 0.887 | 0.980 | 0.947 | 0.877 | 1 |
| HERC6, SLC1A2, IL1R2, FAM20A, RETN | | | | | | | | | |
| RSAD2, IFI27, OAS1, IFI44L, SIGLEC1, | 0.866 | 0.857 | 0.876 | 0.929 | 0.878 | 0.980 | 0.943 | 0.881 | 1 |
| ISG15, HERC6, SLC1A2, OLAH, RETN | | | | | | | | | |
| OAS1, IFIT1, SIGLEC1, ISG15, HERC6, | 0.866 | 0.856 | 0.877 | 0.929 | 0.883 | 0.975 | 0.962 | 0.902 | 1 |
| SLC1A2, IL1R2, OLAH, FAM20A, MMP8 | | | | | | | | | |
| IFI27, OAS1, IFIT1, ISG15, HERC6, | 0.866 | 0.856 | 0.877 | 0.930 | 0.880 | 0.979 | 0.940 | 0.876 | 1 |
| SLC1A2, IL1R2, OLAH, RETN, MMP8 | | | | | | | | | |
| IFIT1, IFI44L, SIGLEC1, ISG15, HERC6, | 0.866 | 0.856 | 0.877 | 0.931 | 0.883 | 0.979 | 0.946 | 0.878 | 1 |
| SLC1A2, OLAH, FAM20A, RETN, MMP8 | | | | | | | | | |
| IFI27, IFIT1, SIGLEC1, ISG15, HERC6, | 0.866 | 0.856 | 0.876 | 0.931 | 0.881 | 0.980 | 0.953 | 0.902 | 1 |
| SLC1A2, IL1R2, OLAH, RETN, MMP8 | | | | | | | | | |
| RSAD2, IFI27, OAS1, IFIT1, IFI44L, | 0.866 | 0.857 | 0.876 | 0.930 | 0.881 | 0.979 | 0.936 | 0.861 | 1 |
| SIGLEC1, ISG15, HERC6, SLC1A2, | | | | | | | | | |
| FAM20A | | | | | | | | | |
| RSAD2, IFIT1, SIGLEC1, ISG15, HERC6, | 0.866 | 0.857 | 0.876 | 0.928 | 0.881 | 0.975 | 0.961 | 0.897 | 1 |
| SLC1A2, ILIR2, OLAH, FAM20A, MMP8 | | | | | | | | | |
| RSAD2, OAS1, IFIT1, IFI44L, SIGLEC1, | 0.866 | 0.856 | 0.876 | 0.921 | 0.863 | 0.979 | 0.949 | 0.890 | 1 |
| HERC6, OLAH, FAM20A, RETN, MMP8 | | | | | | | | | |
| RSAD2, IFIT1, IFI44L, SIGLEC1, HERC6, | 0.866 | 0.856 | 0.877 | 0.926 | 0.871 | 0.980 | 0.962 | 0.907 | 1 |
| IL1R2, OLAH, FAM20A, RETN, MMP8 | | | | | | | | | |
| IFI27, OAS1, IFI44L, ISG15, HERC6, | 0.866 | 0.855 | 0.877 | 0.933 | 0.885 | 0.982 | 0.923 | 0.834 | 1 |
| SLC1A2, IL1R2, FAM20A, RETN, MMP8 | | | | | | | | | |
| RSAD2, IFI27, IFIT1, IFI44L, SIGLEC1, | 0.866 | 0.857 | 0.875 | 0.931 | 0.881 | 0.981 | 0.952 | 0.901 | 1 |
| HERC6, SLC1A2, OLAH, RETN, MMP8 | | | | | | | | | |
| RSAD2, IFI27, OAS1, IFI44L, SIGLEC1, | 0.866 | 0.857 | 0.876 | 0.925 | 0.871 | 0.979 | 0.954 | 0.901 | 1 |
| ISG15, IL1R2, OLAH, RETN, MMP8 | | | | | | | | | |
| IFI27, OAS1, IFI44L, SIGLEC1, ISG15, | 0.866 | 0.856 | 0.876 | 0.930 | 0.880 | 0.979 | 0.937 | 0.873 | 1 |
| HERC6, SLC1A2, IL1R2, OLAH, RETN | | | | | | | | | |
| RSAD2, IFI27, OAS1, IFIT1, IFI44L, | 0.866 | 0.856 | 0.877 | 0.931 | 0.885 | 0.977 | 0.925 | 0.838 | 1 |
| HERC6, SLC1A2, IL1R2, FAM20A, MMP8 | | | | | | | | | |
| RSAD2, IFI27, OAS1, IFIT1, IFI44L, | 0.866 | 0.857 | 0.875 | 0.926 | 0.876 | 0.976 | 0.943 | 0.891 | 0.996 |
| SIGLEC1, ISG15, IL1R2, OLAH, MMP8 | | | | | | | | | |
| IFI27, OAS1, IFIT1, ISG15, HERC6, | 0.866 | 0.856 | 0.876 | 0.933 | 0.884 | 0.981 | 0.925 | 0.842 | 1 |
| SLC1A2, IL1R2, FAM20A, RETN, MMP8 | | | | | | | | | |
| OAS1, IFIT1, SIGLEC1, HERC6, SLC1A2, | 0.866 | 0.856 | 0.876 | 0.930 | 0.883 | 0.977 | 0.958 | 0.894 | 1 |
| ILIR2, OLAH, FAM20A, RETN, MMP8 | | | | | | | | | |
| RSAD2, OASI, IFIT1, SIGLEC1, ISG15, | 0.866 | 0.856 | 0.876 | 0.921 | 0.864 | 0.979 | 0.953 | 0.898 | 1 |
| HERC6, OLAH, FAM20A, RETN, MMP8 | | | | | | | | | |
| OAS1, IFIT1, IFI44L, SIGLEC1, HERC6, | 0.866 | 0.855 | 0.877 | 0.930 | 0.881 | 0.979 | 0.940 | 0.873 | 1 |
| SLC1A2, OLAH, FAM20A, RETN, MMP8 | | | | | | | | | |
| RSAD2, IFI27, OAS1, IFIT1, IFI44L, ISG15, | 0.866 | 0.855 | 0.877 | 0.929 | 0.876 | 0.982 | 0.933 | 0.854 | 1 |
| IL1R2, FAM20A, RETN, MMP8 | | | | | | | | | |
| RSAD2, IFI27, OAS1, IFIT1, HERC6, | 0.866 | 0.856 | 0.876 | 0.928 | 0.877 | 0.979 | 0.940 | 0.875 | 1 |
| SLC1A2, IL1R2, OLAH, RETN, MMP8 | | | | | | | | | |
| RSAD2, IFIT1, IFI44L, SIGLEC1, ISG15, | 0.866 | 0.855 | 0.877 | 0.929 | 0.88 | 0.978 | 0.937 | 0.866 | 1 |
| HERC6, SLC1A2, OLAH, FAM20A, MMP8 | | | | | | | | | |
| RSAD2, IFI27, OAS1, IFIT1, SIGLEC1, | 0.866 | 0.856 | 0.876 | 0.923 | 0.868 | 0.978 | 0.959 | 0.910 | 1 |
| ISG15, IL1R2, OLAH, RETN, MMP8 | | | | | | | | | |
| RSAD2, IFI27, OAS1, IFIT1, IFI44L, | 0.866 | 0.855 | 0.876 | 0.927 | 0.876 | 0.978 | 0.935 | 0.859 | 1 |
| SIGLEC1, HERC6, SLC1A2, FAM20A, | | | | | | | | | |
| MMP8 | | | | | | | | | |
| OAS1, IFIT1, IFI44L, SIGLEC1, ISG15, | 0.866 | 0.855 | 0.877 | 0.929 | 0.880 | 0.978 | 0.938 | 0.868 | 1 |
| HERC6, SLC1A2, OLAH, FAM20A, MMP8 | | | | | | | | | |
| OAS1, IFIT1, IFI44L, SIGLEC1, HERC6, | 0.866 | 0.856 | 0.876 | 0.929 | 0.882 | 0.975 | 0.958 | 0.892 | 1 |
| SLC1A2, IL1R2, FAM20A, RETN, MMP8 | | | | | | | | | |
| RSAD2, IFI27, SIGLEC1, ISG15, HERC6, | 0.866 | 0.856 | 0.876 | 0.931 | 0.884 | 0.979 | 0.950 | 0.897 | 1 |
| SLC1A2, IL1R2, OLAH, RETN, MMP8 | | | | | | | | | |
| IFI27, IFI44L, SIGLEC1, ISG15, HERC6, | 0.866 | 0.856 | 0.875 | 0.930 | 0.882 | 0.979 | 0.949 | 0.896 | 1 |
| SLC1A2, IL1R2, OLAH, RETN, MMP8 | | | | | | | | | |
| RSAD2, IFI27, OAS1, IFIT1, IFI44L, | 0.866 | 0.857 | 0.875 | 0.924 | 0.870 | 0.978 | 0.940 | 0.876 | 1 |
| SIGLEC1, ISG15, HERC6, SLC1A2, RETN | | | | | | | | | |
| RSAD2, OAS1, SIGLEC1, ISG15, HERC6, | 0.866 | 0.856 | 0.876 | 0.918 | 0.861 | 0.975 | 0.961 | 0.903 | 1 |
| IL1R2, OLAH, FAM20A, RETN, MMP8 | | | | | | | | | |
| RSAD2, IFI27, OAS1, IFIT1, IFI44L, ISG15, | 0.866 | 0.856 | 0.875 | 0.924 | 0.873 | 0.974 | 0.939 | 0.883 | 0.995 |
| HERC6, IL1R2, OLAH, MMP8 | | | | | | | | | |
| RSAD2, OAS1, IFI44L, SIGLEC1, ISG15, | 0.866 | 0.856 | 0.876 | 0.919 | 0.863 | 0.976 | 0.962 | 0.905 | 1 |
| HERC6, IL1R2, OLAH, FAM20A, MMP8 | | | | | | | | | |
| RSAD2, IFI27, OAS1, IFIT1, IFI44L, | 0.866 | 0.856 | 0.875 | 0.931 | 0.882 | 0.980 | 0.943 | 0.886 | 1 |
| SIGLEC1, ISG15, HERC6, SLC1A2, OLAH | | | | | | | | | |
| RSAD2, IFI27, OAS1, IFIT1, IFI44L, | 0.866 | 0.857 | 0.875 | 0.922 | 0.867 | 0.977 | 0.949 | 0.893 | 1 |
| SIGLEC1, ISG15, IL1R2, OLAH, RETN | | | | | | | | | |
| RSAD2, IFI27, OAS1, IFI44L, SIGLEC1, | 0.866 | 0.855 | 0.876 | 0.928 | 0.875 | 0.981 | 0.937 | 0.861 | 1 |
| ISG15, HERC6, SLC1A2, FAM20A, RETN | | | | | | | | | |
| IFI27, IFIT1, IFI44L, SIGLEC1, HERC6, | 0.866 | 0.856 | 0.875 | 0.929 | 0.880 | 0.979 | 0.955 | 0.907 | 1 |
| SLC1A2, IL1R2, OLAH, RETN, MMP8 | | | | | | | | | |
| OAS1, IFIT1, IFI44L, SIGLEC1, ISG15, | 0.866 | 0.855 | 0.876 | 0.920 | 0.863 | 0.978 | 0.968 | 0.918 | 1 |
| IL1R2, OLAH, FAM20A, RETN, MMP8 | | | | | | | | | |
| IFI27, OAS1, IFIT1, SIGLEC1, ISG15, | 0.866 | 0.855 | 0.876 | 0.929 | 0.880 | 0.977 | 0.941 | 0.884 | 0.999 |
| HERC6, SLC1A2, IL1R2, OLAH, MMP8 | | | | | | | | | |
| IFIT1, IFI44L, SIGLEC1, ISG15, HERC6, | 0.866 | 0.856 | 0.875 | 0.929 | 0.883 | 0.976 | 0.958 | 0.890 | 1 |
| SLC1A2, IL1R2, FAM20A, RETN, MMP8 | | | | | | | | | |
| IFI27, OAS1, IFIT1, IFI44L, SIGLEC1, | 0.866 | 0.856 | 0.875 | 0.927 | 0.880 | 0.974 | 0.954 | 0.905 | 1 |
| HERC6, SLC1A2, IL1R2, RETN, MMP8 | | | | | | | | | |
| RSAD2, IFI27, IFIT1, IFI44L, HERC6, | 0.865 | 0.856 | 0.875 | 0.933 | 0.885 | 0.981 | 0.929 | 0.847 | 1 |
| SLC1A2, IL1R2, FAM20A, RETN, MMP8 | | | | | | | | | |
| IFI27, OAS1, IFIT1, IFI44L, SIGLEC1, | 0.865 | 0.855 | 0.876 | 0.930 | 0.880 | 0.979 | 0.941 | 0.884 | 0.999 |
| ISG15, HERC6, SLC1A2, OLAH, MMP8 | | | | | | | | | |
| RSAD2, IFI27, OAS1, IFIT1, HERC6, | 0.865 | 0.855 | 0.876 | 0.933 | 0.884 | 0.981 | 0.926 | 0.843 | 1 |
| SLC1A2, IL1R2, FAM20A, RETN, MMP8 | | | | | | | | | |
| RSAD2, OAS1, IFIT1, SIGLEC1, ISG15, | 0.865 | 0.856 | 0.875 | 0.920 | 0.863 | 0.978 | 0.968 | 0.918 | 1 |
| IL1R2, OLAH, FAM20A, RETN, MMP8 | | | | | | | | | |
| RSAD2, IFI27, OAS1, ISG15, HERC6, | 0.865 | 0.855 | 0.875 | 0.930 | 0.879 | 0.981 | 0.940 | 0.875 | 1 |
| SLC1A2, IL1R2, OLAH, RETN, MMP8 | | | | | | | | | |
| RSAD2, IFI27, IFIT1, IFI44L, SIGLEC1, | 0.865 | 0.856 | 0.875 | 0.928 | 0.879 | 0.977 | 0.943 | 0.888 | 0.999 |
| HERC6, SLC1A2, IL1R2, OLAH, RETN | | | | | | | | | |
| IFIT1, SIGLEC1, ISG15, HERC6, SLC1A2, | 0.865 | 0.855 | 0.875 | 0.929 | 0.881 | 0.976 | 0.959 | 0.893 | 1 |
| IL1R2, OLAH, FAM20A, RETN, MMP8 | | | | | | | | | |
| IFIT1, IFI44L, SIGLEC1, HERC6, SLC1A2, | 0.865 | 0.855 | 0.876 | 0.931 | 0.884 | 0.978 | 0.960 | 0.899 | 1 |
| IL1R2, OLAH, FAM20A, RETN, MMP8 | | | | | | | | | |
| RSAD2, IFI27, OAS1, IFIT1, IFI44L, | 0.865 | 0.855 | 0.875 | 0.929 | 0.875 | 0.983 | 0.921 | 0.839 | 1 |
| SLC1A2, ILIR2, OLAH, FAM20A, RETN | | | | | | | | | |
| IFI27, OAS1, IFIT1, SIGLEC1, ISG15, | 0.865 | 0.855 | 0.875 | 0.930 | 0.881 | 0.980 | 0.953 | 0.898 | 1 |
| SLC1A2, IL1R2, OLAH, RETN, MMP8 | | | | | | | | | |
| RSAD2, IFI27, OAS1, IFIT1, IFI44L, ISG15, | 0.865 | 0.855 | 0.876 | 0.928 | 0.874 | 0.983 | 0.920 | 0.838 | 1 |
| SLC1A2, OLAH, FAM20A, RETN | | | | | | | | | |
| RSAD2, IFI27, IFI44L, SIGLEC1, ISG15, | 0.865 | 0.856 | 0.874 | 0.928 | 0.878 | 0.978 | 0.943 | 0.887 | 1 |
| HERC6, SLC1A2, IL1R2, OLAH, RETN | | | | | | | | | |
| RSAD2, IFI27, OAS1, IFIT1, SIGLEC1, | 0.865 | 0.855 | 0.875 | 0.929 | 0.880 | 0.977 | 0.945 | 0.888 | 1 |
| HERC6, SLC1A2, IL1R2, OLAH, MMP8 | | | | | | | | | |
| IFI27, OAS1, IFIT1, SIGLEC1, ISG15, | 0.865 | 0.856 | 0.875 | 0.928 | 0.881 | 0.975 | 0.955 | 0.903 | 1 |
| HERC6, SLC1A2, IL1R2, RETN, MMP8 | | | | | | | | | |
| RSAD2, IFI27, OAS1, IFIT1, IFI44L, | 0.865 | 0.855 | 0.875 | 0.926 | 0.872 | 0.981 | 0.938 | 0.864 | 1 |
| SIGLEC1, ISG15, SLC1A2, FAM20A, RETN | | | | | | | | | |
| RSAD2, IFI27, OAS1, IFI44L, SIGLEC1, | 0.865 | 0.856 | 0.875 | 0.930 | 0.881 | 0.979 | 0.945 | 0.890 | 1.000 |
| ISG15, HERC6, SLC1A2, IL1R2, OLAH | | | | | | | | | |
| RSAD2, IFI27, OAS1, IFIT1, IFI44L, | 0.865 | 0.855 | 0.875 | 0.928 | 0.877 | 0.980 | 0.940 | 0.875 | 1 |
| HERC6, SLC1A2, OLAH, RETN, MMP8 | | | | | | | | | |
| RSAD2, OAS1, IFIT1, IFI44L, SIGLEC1, | 0.865 | 0.855 | 0.875 | 0.926 | 0.874 | 0.977 | 0.941 | 0.877 | 1 |
| ISG15, SLC1A2, OLAH, FAM20A, MMP8 | | | | | | | | | |
| RSAD2, IFI27, IFIT1, SIGLEC1, ISG15, | 0.865 | 0.855 | 0.875 | 0.931 | 0.882 | 0.980 | 0.949 | 0.895 | 1 |
| HERC6, SLC1A2, OLAH, RETN, MMP8 | | | | | | | | | |
| RSAD2, IFI27, OAS1, IFIT1, IFI44L, | 0.865 | 0.855 | 0.875 | 0.929 | 0.878 | 0.979 | 0.945 | 0.889 | 1 |
| SIGLEC1, HERC6, SLC1A2, OLAH, MMP8 | | | | | | | | | |
| RSAD2, OAS1, IFIT1, SIGLEC1, ISG15, | 0.865 | 0.854 | 0.876 | 0.926 | 0.877 | 0.975 | 0.943 | 0.874 | 1 |
| HERC6, SLC1A2, OLAH, FAM20A, MMP8 | | | | | | | | | |
| IFI27, OAS1, IFIT1, IFI44L, HERC6, | 0.865 | 0.855 | 0.875 | 0.929 | 0.878 | 0.979 | 0.939 | 0.874 | 1 |
| SLC1A2, IL1R2, OLAH, RETN, MMP8 | | | | | | | | | |
| RSAD2, IFI27, IFIT1, IFI44L, HERC6, | 0.865 | 0.855 | 0.875 | 0.928 | 0.877 | 0.979 | 0.941 | 0.881 | 1 |
| SLC1A2, IL1R2, OLAH, RETN, MMP8 | | | | | | | | | |
| OAS1, IFIT1, SIGLEC1, ISG15, HERC6, | 0.865 | 0.855 | 0.875 | 0.929 | 0.882 | 0.975 | 0.957 | 0.889 | 1 |
| SLC1A2, IL1R2, FAM20A, RETN, MMP8 | | | | | | | | | |
| RSAD2, IFI27, OAS1, IFIT1, SIGLEC1, | 0.865 | 0.855 | 0.875 | 0.930 | 0.880 | 0.980 | 0.940 | 0.88 | 0.999 |
| ISG15, HERC6, SLC1A2, OLAH, MMP8 | | | | | | | | | |
| RSAD2, IFI27, IFIT1, IFI44L, SIGLEC1, | 0.865 | 0.856 | 0.874 | 0.923 | 0.870 | 0.976 | 0.953 | 0.908 | 0.999 |
| ISG15, HERC6, IL1R2, RETN, MMP8 | | | | | | | | | |
| OAS1, IFIT1, SIGLEC1, ISG15, HERC6, | 0.865 | 0.855 | 0.875 | 0.926 | 0.876 | 0.976 | 0.947 | 0.881 | 1 |
| SLC1A2, OLAH, FAM20A, RETN, MMP8 | | | | | | | | | |
| RSAD2, IFIT1, IFI44L, SIGLEC1, ISG15, | 0.865 | 0.855 | 0.875 | 0.927 | 0.879 | 0.975 | 0.960 | 0.900 | 1 |
| SLC1A2, IL1R2, OLAH, FAM20A, MMP8 | | | | | | | | | |
| RSAD2, IFI27, IFIT1, IFI44L, SIGLEC1, | 0.865 | 0.854 | 0.876 | 0.927 | 0.872 | 0.983 | 0.942 | 0.869 | 1 |
| ISG15, SLC1A2, FAM20A, RETN, MMP8 | | | | | | | | | |
| RSAD2, IFIT1, IFI44L, SIGLEC1, ISG15, | 0.865 | 0.855 | 0.875 | 0.922 | 0.865 | 0.979 | 0.953 | 0.896 | 1 |
| HERC6, OLAH, FAM20A, RETN, MMP8 | | | | | | | | | |
| RSAD2, OAS1, IFIT1, IFI44L, SIGLEC1, | 0.865 | 0.855 | 0.875 | 0.926 | 0.878 | 0.975 | 0.959 | 0.904 | 1 |
| SLC1A2, IL1R2, OLAH, FAM20A, MMP8 | | | | | | | | | |
| RSAD2, OAS1, IFIT1, SIGLEC1, ISG15, | 0.865 | 0.855 | 0.874 | 0.926 | 0.877 | 0.974 | 0.963 | 0.904 | 1 |
| SLC1A2, IL1R2, OLAH, FAM20A, MMP8 | | | | | | | | | |
| RSAD2, IFI27, OAS1, IFIT1, SIGLEC1, | 0.865 | 0.855 | 0.875 | 0.929 | 0.879 | 0.978 | 0.937 | 0.862 | 1 |
| ISG15, HERC6, SLC1A2, FAM20A, MMP8 | | | | | | | | | |
| RSAD2, IFI27, OAS1, IFIT1, SIGLEC1, | 0.865 | 0.854 | 0.875 | 0.926 | 0.870 | 0.982 | 0.943 | 0.868 | 1 |
| ISG15, SLC1A2, FAM20A, RETN, MMP8 | | | | | | | | | |
| RSAD2, IFI27, IFIT1, ISG15, HERC6, | 0.865 | 0.855 | 0.874 | 0.933 | 0.885 | 0.982 | 0.932 | 0.849 | 1 |
| SLC1A2, IL1R2, FAM20A, RETN, MMP8 | | | | | | | | | |
| IFI27, OAS1, IFIT1, IFI44L, SIGLEC1, | 0.865 | 0.856 | 0.874 | 0.929 | 0.879 | 0.979 | 0.943 | 0.886 | 1 |
| ISG15, HERC6, SLC1A2, RETN, MMP8 | | | | | | | | | |
| RSAD2, IFI27, IFI44L, SIGLEC1, ISG15, | 0.865 | 0.854 | 0.876 | 0.930 | 0.877 | 0.983 | 0.946 | 0.872 | 1 |
| HERC6, SLC1A2, FAM20A, RETN, MMP8 | | | | | | | | | |
| RSAD2, IFI27, OAS1, ISG15, HERC6, | 0.865 | 0.855 | 0.875 | 0.934 | 0.887 | 0.982 | 0.924 | 0.836 | 1 |
| SLC1A2, IL1R2, FAM20A, RETN, MMP8 | | | | | | | | | |
| RSAD2, IFI27, OAS1, IFI44L, SIGLEC1, | 0.865 | 0.855 | 0.874 | 0.931 | 0.881 | 0.981 | 0.942 | 0.885 | 1 |
| ISG15, HERC6, SLC1A2, OLAH, MMP8 | | | | | | | | | |
| IFI27, IFIT1, IFI44L, SIGLEC1, ISG15, | 0.865 | 0.855 | 0.875 | 0.930 | 0.880 | 0.980 | 0.951 | 0.899 | 1 |
| HERC6, SLC1A2, OLAH, RETN, MMP8 | | | | | | | | | |
| RSAD2, OAS1, IFIT1, IFI44L, SIGLEC1, | 0.865 | 0.854 | 0.875 | 0.922 | 0.865 | 0.978 | 0.967 | 0.916 | 1 |
| IL1R2, OLAH, FAM20A, RETN, MMP8 | | | | | | | | | |
| RSAD2, IFI27, OAS1, IFIT1, IFI44L, | 0.865 | 0.854 | 0.875 | 0.927 | 0.872 | 0.982 | 0.943 | 0.871 | 1 |
| SIGLEC1, SLC1A2, FAM20A, RETN, | | | | | | | | | |
| MMP8 | | | | | | | | | |
| RSAD2, OAS1, IFIT1, IFI44L, SIGLEC1, | 0.865 | 0.854 | 0.876 | 0.917 | 0.859 | 0.975 | 0.949 | 0.891 | 1 |
| ISG15, HERC6, OLAH, FAM20A, MMP8 | | | | | | | | | |
| RSAD2, IFI27, OAS1, SIGLEC1, ISG15, | 0.865 | 0.854 | 0.875 | 0.929 | 0.880 | 0.978 | 0.942 | 0.884 | 1 |
| HERC6, SLC1A2, IL1R2, OLAH, MMP8 | | | | | | | | | |
| IFI27, OAS1, IFIT1, IFI44L, SIGLEC1, | 0.865 | 0.855 | 0.875 | 0.930 | 0.882 | 0.978 | 0.940 | 0.883 | 0.997 |
| ISG15, HERC6, SLC1A2, ILIR2, OLAH | | | | | | | | | |
| RSAD2, IFI27, IFIT1, IFI44L, SIGLEC1, | 0.865 | 0.856 | 0.873 | 0.927 | 0.877 | 0.978 | 0.941 | 0.884 | 0.998 |
| ISG15, HERC6, SLC1A2, OLAH, RETN | | | | | | | | | |
| OAS1, IFIT1, IFI44L, SIGLEC1, ISG15, | 0.865 | 0.854 | 0.875 | 0.928 | 0.883 | 0.974 | 0.961 | 0.899 | 1 |
| HERC6, SLC1A2, IL1R2, FAM20A, MMP8 | | | | | | | | | |
| RSAD2, IFI27, IFIT1, SIGLEC1, ISG15, | 0.865 | 0.855 | 0.874 | 0.929 | 0.878 | 0.979 | 0.940 | 0.882 | 0.998 |
| HERC6, SLC1A2, IL1R2, OLAH, RETN | | | | | | | | | |
| RSAD2, IFI27, IFIT1, ISG15, HERC6, | 0.865 | 0.855 | 0.874 | 0.930 | 0.880 | 0.979 | 0.939 | 0.878 | 1 |
| SLC1A2, IL1R2, OLAH, RETN, MMP8 | | | | | | | | | |
| RSAD2, OAS1, SIGLEC1, ISG15, HERC6, | 0.865 | 0.854 | 0.875 | 0.924 | 0.876 | 0.972 | 0.961 | 0.901 | 1 |
| SLC1A2, IL1R2, OLAH, FAM20A, MMP8 | | | | | | | | | |
| RSAD2, IFI27, OAS1, IFI44L, HERC6, | 0.865 | 0.854 | 0.875 | 0.933 | 0.884 | 0.981 | 0.923 | 0.834 | 1 |
| SLC1A2, IL1R2, FAM20A, RETN, MMP8 | | | | | | | | | |
| RSAD2, OAS1, IFIT1, SIGLEC1, HERC6, | 0.865 | 0.854 | 0.875 | 0.927 | 0.877 | 0.977 | 0.948 | 0.881 | 1 |
| SLC1A2, OLAH, FAM20A, RETN, MMP8 | | | | | | | | | |
| IFI27, OAS1, IFIT1, IFI44L, ISG15, HERC6, | 0.864 | 0.854 | 0.875 | 0.933 | 0.885 | 0.980 | 0.925 | 0.840 | 1 |
| SLC1A2, IL1R2, FAM20A, RETN | | | | | | | | | |
| RSAD2, OAS1, IFIT1, IFI44L, SIGLEC1, | 0.864 | 0.854 | 0.875 | 0.928 | 0.882 | 0.974 | 0.963 | 0.904 | 1 |
| HERC6, SLC1A2, IL1R2, FAM20A, MMP8 | | | | | | | | | |
| RSAD2, IFI27, OAS1, IFI44L, SIGLEC1, | 0.864 | 0.855 | 0.874 | 0.928 | 0.880 | 0.976 | 0.950 | 0.897 | 1 |
| HERC6, SLC1A2, IL1R2, OLAH, MMP8 | | | | | | | | | |
| RSAD2, IFI27, OAS1, IFIT1, IFI44L, | 0.864 | 0.855 | 0.873 | 0.925 | 0.873 | 0.977 | 0.95 | 0.887 | 1 |
| SIGLEC1, SLC1A2, IL1R2, OLAH, RETN | | | | | | | | | |
| RSAD2, IFI27, OAS1, IFIT1, IFI44L, | 0.864 | 0.856 | 0.873 | 0.928 | 0.879 | 0.976 | 0.945 | 0.882 | 1 |
| SIGLEC1, HERC6, SLC1A2, IL1R2, RETN | | | | | | | | | |
| RSAD2, IFI27, OAS1, IFIT1, IFI44L, ISG15, | 0.864 | 0.855 | 0.874 | 0.924 | 0.870 | 0.978 | 0.940 | 0.879 | 1 |
| HERC6, IL1R2, RETN, MMP8 | | | | | | | | | |
| RSAD2, IFI27, OAS1, IFIT1, SIGLEC1, | 0.864 | 0.855 | 0.874 | 0.929 | 0.879 | 0.979 | 0.955 | 0.900 | 1 |
| SLC1A2, IL1R2, OLAH, RETN, MMP8 | | | | | | | | | |
| RSAD2, IFI27, OAS1, IFIT1, IFI44L, | 0.864 | 0.855 | 0.874 | 0.929 | 0.880 | 0.977 | 0.950 | 0.897 | 1 |
| SIGLEC1, HERC6, SLC1A2, IL1R2, OLAH | | | | | | | | | |
| RSAD2, IFI27, OAS1, IFI44L, SIGLEC1, | 0.864 | 0.855 | 0.873 | 0.930 | 0.880 | 0.980 | 0.947 | 0.883 | 1 |
| ISG15, SLC1A2, IL1R2, OLAH, RETN | | | | | | | | | |
| RSAD2, IFI27, IFI44L, SIGLEC1, ISG15, | 0.864 | 0.854 | 0.874 | 0.931 | 0.88 | 0.980 | 0.951 | 0.899 | 1 |
| HERC6, SLC1A2, OLAH, RETN, MMP8 | | | | | | | | | |
| RSAD2, IFI27, OAS1, IFIT1, ISG15, HERC6, | 0.864 | 0.854 | 0.874 | 0.928 | 0.876 | 0.979 | 0.932 | 0.860 | 1 |
| SLC1A2, IL1R2, OLAH, RETN | | | | | | | | | |
| RSAD2, IFI27, IFIT1, IFI44L, SIGLEC1, | 0.864 | 0.854 | 0.874 | 0.927 | 0.877 | 0.978 | 0.937 | 0.860 | 1 |
| ISG15, HERC6, SLC1A2, FAM20A, MMP8 | | | | | | | | | |
| IFI27, OAS1, IFIT1, IFI44L, ISG15, HERC6, | 0.864 | 0.854 | 0.875 | 0.930 | 0.879 | 0.980 | 0.939 | 0.874 | 1 |
| SLC1A2, OLAH, RETN, MMP8 | | | | | | | | | |
| IFI27, OAS1, IFIT1, IFI44L, SIGLEC1, | 0.864 | 0.855 | 0.873 | 0.928 | 0.881 | 0.975 | 0.941 | 0.883 | 0.999 |
| ISG15, HERC6, SLC1A2, IL1R2, RETN | | | | | | | | | |
| OAS1, IFIT1, IFI44L, SIGLEC1, ISG15, | 0.864 | 0.854 | 0.874 | 0.928 | 0.880 | 0.975 | 0.961 | 0.903 | 1 |
| SLC1A2, IL1R2, OLAH, FAM20A, MMP8 | | | | | | | | | |
| IFI27, OAS1, IFIT1, IFI44L, SIGLEC1, | 0.864 | 0.854 | 0.874 | 0.927 | 0.878 | 0.975 | 0.946 | 0.891 | 1 |
| HERC6, SLC1A2, IL1R2, OLAH, MMP8 | | | | | | | | | |
| RSAD2, IFIT1, SIGLEC1, HERC6, SLC1A2, | 0.864 | 0.854 | 0.874 | 0.929 | 0.881 | 0.976 | 0.955 | 0.889 | 1 |
| IL1R2, OLAH, FAM20A, RETN, MMP8 | | | | | | | | | |
| RSAD2, IFI27, OAS1, IFIT1, SIGLEC1, | 0.864 | 0.855 | 0.873 | 0.924 | 0.870 | 0.978 | 0.940 | 0.878 | 1 |
| ISG15, HERC6, SLC1A2, RETN, MMP8 | | | | | | | | | |
| RSAD2, IFI27, OAS1, IFIT1, SIGLEC1, | 0.864 | 0.855 | 0.874 | 0.929 | 0.882 | 0.977 | 0.957 | 0.903 | 1 |
| HERC6, SLC1A2, IL1R2, RETN, MMP8 | | | | | | | | | |
| OAS1, IFIT1, IFI44L, SIGLEC1, ISG15, | 0.864 | 0.854 | 0.874 | 0.918 | 0.86 | 0.976 | 0.952 | 0.897 | 1 |
| HERC6, IL1R2, OLAH, FAM20A, RETN | | | | | | | | | |
| IFI27, OAS1, IFI44L, SIGLEC1, ISG15, | 0.864 | 0.854 | 0.874 | 0.930 | 0.882 | 0.978 | 0.941 | 0.886 | 0.997 |
| HERC6, SLC1A2, IL1R2, OLAH, MMP8 | | | | | | | | | |
| IFIT1, IFI44L, SIGLEC1, ISG15, SLC1A2, | 0.864 | 0.855 | 0.874 | 0.926 | 0.878 | 0.975 | 0.960 | 0.900 | 1 |
| ILIR2, OLAH, FAM20A, RETN, MMP8 | | | | | | | | | |
| RSAD2, OAS1, IFIT1, SIGLEC1, ISG15, | 0.864 | 0.855 | 0.874 | 0.919 | 0.861 | 0.977 | 0.954 | 0.900 | 1 |
| HERC6, IL1R2, OLAH, FAM20A, RETN | | | | | | | | | |
| IFI27, IFIT1, IFI44L, ISG15, HERC6, | 0.864 | 0.854 | 0.874 | 0.931 | 0.882 | 0.980 | 0.930 | 0.847 | 1 |
| SLC1A2, IL1R2, FAM20A, RETN, MMP8 | | | | | | | | | |
| IFI27, OAS1, IFIT1, IFI44L, ISG15, HERC6, | 0.864 | 0.853 | 0.875 | 0.931 | 0.885 | 0.978 | 0.926 | 0.840 | 1 |
| SLC1A2, IL1R2, FAM20A, MMP8 | | | | | | | | | |
| RSAD2, OAS1, IFIT1, SIGLEC1, ISG15, | 0.864 | 0.854 | 0.874 | 0.927 | 0.881 | 0.974 | 0.960 | 0.896 | 1 |
| HERC6, SLC1A2, ILIR2, FAM20A, MMP8 | | | | | | | | | |
| RSAD2, OAS1, IFI44L, SIGLEC1, HERC6, | 0.864 | 0.853 | 0.875 | 0.923 | 0.875 | 0.972 | 0.959 | 0.897 | 1 |
| SLC1A2, IL1R2, OLAH, FAM20A, MMP8 | | | | | | | | | |
| RSAD2, IFI27, OAS1, IFI44L, HERC6, | 0.864 | 0.854 | 0.874 | 0.929 | 0.879 | 0.980 | 0.941 | 0.877 | 1 |
| SLC1A2, IL1R2, OLAH, RETN, MMP8 | | | | | | | | | |
| RSAD2, IFI27, OAS1, IFIT1, IFI44L, | 0.864 | 0.855 | 0.873 | 0.929 | 0.878 | 0.979 | 0.947 | 0.890 | 1 |
| SIGLEC1, ISG15, SLC1A2, IL1R2, OLAH | | | | | | | | | |
| RSAD2, IFI27, OAS1, IFIT1, SIGLEC1, | 0.864 | 0.855 | 0.873 | 0.926 | 0.877 | 0.975 | 0.947 | 0.886 | 1 |
| ISG15, HERC6, SLC1A2, IL1R2, RETN | | | | | | | | | |
| RSAD2, IFI27, OAS1, IFIT1, ISG15, HERC6, | 0.864 | 0.853 | 0.875 | 0.928 | 0.876 | 0.980 | 0.940 | 0.875 | 1 |
| SLC1A2, OLAH, RETN, MMP8 | | | | | | | | | |
| RSAD2, IFI27, OAS1, IFIT1, SIGLEC1, | 0.864 | 0.855 | 0.873 | 0.929 | 0.880 | 0.979 | 0.945 | 0.887 | 1 |
| ISG15, SLC1A2, IL1R2, OLAH, MMP8 | | | | | | | | | |
| RSAD2, IFI27, OAS1, IFIT1, IFI44L, ISG15, | 0.864 | 0.854 | 0.874 | 0.919 | 0.861 | 0.976 | 0.949 | 0.895 | 1 |
| ILIR2, OLAH, RETN, MMP8 | | | | | | | | | |
| RSAD2, IFI27, OAS1, IFI44L, ISG15, | 0.864 | 0.854 | 0.874 | 0.928 | 0.877 | 0.979 | 0.932 | 0.860 | 1 |
| HERC6, SLC1A2, IL1R2, OLAH, RETN | | | | | | | | | |
| RSAD2, IFI27, IFIT1, SIGLEC1, ISG15, | 0.864 | 0.854 | 0.873 | 0.929 | 0.880 | 0.978 | 0.941 | 0.885 | 0.997 |
| HERC6, SLC1A2, IL1R2, OLAH, MMP8 | | | | | | | | | |
| RSAD2, IFI27, OAS1, IFI44L, SIGLEC1, | 0.864 | 0.855 | 0.872 | 0.927 | 0.879 | 0.976 | 0.946 | 0.884 | 1 |
| ISG15, HERC6, SLC1A2, IL1R2, RETN | | | | | | | | | |
| IFI27, OAS1, IFIT1, IFI44L, SIGLEC1, | 0.864 | 0.854 | 0.874 | 0.927 | 0.876 | 0.978 | 0.952 | 0.896 | 1 |
| SLC1A2, IL1R2, OLAH, RETN, MMP8 | | | | | | | | | |
| RSAD2, IFI27, OAS1, IFI44L, ISG15, | 0.864 | 0.853 | 0.875 | 0.930 | 0.884 | 0.976 | 0.923 | 0.834 | 1 |
| HERC6, SLC1A2, ILIR2, FAM20A, MMP8 | | | | | | | | | |
| RSAD2, IFI27, OAS1, IFIT1, IFI44L, | 0.864 | 0.855 | 0.873 | 0.930 | 0.880 | 0.981 | 0.948 | 0.893 | 1 |
| SIGLEC1, ISG15, SLC1A2, OLAH, MMP8 | | | | | | | | | |
| RSAD2, IFI27, OAS1, IFI44L, ISG15, | 0.864 | 0.854 | 0.874 | 0.930 | 0.880 | 0.981 | 0.940 | 0.875 | 1 |
| HERC6, SLC1A2, OLAH, RETN, MMP8 | | | | | | | | | |
| RSAD2, OAS1, IFIT1, IFI44L, SIGLEC1, | 0.864 | 0.854 | 0.874 | 0.921 | 0.863 | 0.979 | 0.946 | 0.881 | 1 |
| ISG15, HERC6, FAM20A, RETN, MMP8 | | | | | | | | | |
| OAS1, IFI44L, SIGLEC1, ISG15, HERC6, | 0.864 | 0.854 | 0.874 | 0.918 | 0.861 | 0.975 | 0.961 | 0.903 | 1 |
| IL1R2, OLAH, FAM20A, RETN, MMP8 | | | | | | | | | |
| RSAD2, IFI27, OAS1, IFIT1, IFI44L, | 0.864 | 0.854 | 0.874 | 0.927 | 0.875 | 0.978 | 0.932 | 0.860 | 1 |
| HERC6, SLC1A2, IL1R2, OLAH, RETN | | | | | | | | | |
| RSAD2, IFI27, OAS1, IFIT1, IFI44L, | 0.864 | 0.855 | 0.873 | 0.929 | 0.880 | 0.977 | 0.951 | 0.896 | 1 |
| SIGLEC1, SLC1A2, IL1R2, OLAH, MMP8 | | | | | | | | | |
| OAS1, IFI44L, SIGLEC1, ISG15, HERC6, | 0.864 | 0.853 | 0.874 | 0.922 | 0.872 | 0.972 | 0.961 | 0.901 | 1 |
| SLC1A2, IL1R2, OLAH, FAM20A, MMP8 | | | | | | | | | |
| IFI27, OAS1, IFI44L, ISG15, HERC6, | 0.864 | 0.853 | 0.874 | 0.930 | 0.881 | 0.979 | 0.937 | 0.871 | 1 |
| SLC1A2, IL1R2, OLAH, RETN, MMP8 | | | | | | | | | |
| RSAD2, IFIT1, IFI44L, SIGLEC1, HERC6, | 0.864 | 0.853 | 0.874 | 0.931 | 0.883 | 0.979 | 0.940 | 0.872 | 1 |
| SLC1A2, OLAH, FAM20A, RETN, MMP8 | | | | | | | | | |
| RSAD2, IFI27, OAS1, IFI44L, SIGLEC1, | 0.864 | 0.854 | 0.873 | 0.930 | 0.881 | 0.979 | 0.949 | 0.895 | 1 |
| ISG15, SLC1A2, IL1R2, OLAH, MMP8 | | | | | | | | | |
| RSAD2, IFI27, OAS1, IFIT1, IFI44L, | 0.864 | 0.855 | 0.873 | 0.926 | 0.873 | 0.979 | 0.941 | 0.876 | 1 |
| SIGLEC1, HERC6, SLC1A2, RETN, MMP8 | | | | | | | | | |
| RSAD2, IFIT1, SIGLEC1, ISG15, HERC6, | 0.864 | 0.854 | 0.873 | 0.927 | 0.878 | 0.977 | 0.946 | 0.878 | 1 |
| SLC1A2, OLAH, FAM20A, RETN, MMP8 | | | | | | | | | |
| RSAD2, IFIT1, IFI44L, SIGLEC1, ISG15, | 0.864 | 0.854 | 0.874 | 0.928 | 0.882 | 0.973 | 0.959 | 0.891 | 1 |
| HERC6, SLC1A2, IL1R2, FAM20A, MMP8 | | | | | | | | | |
| RSAD2, IFI27, IFIT1, SIGLEC1, ISG15, | 0.864 | 0.854 | 0.873 | 0.930 | 0.881 | 0.979 | 0.953 | 0.900 | 1 |
| SLC1A2, IL1R2, OLAH, RETN, MMP8 | | | | | | | | | |
| RSAD2, IFIT1, IFI44L, SIGLEC1, HERC6, | 0.864 | 0.854 | 0.873 | 0.930 | 0.883 | 0.976 | 0.957 | 0.891 | 1 |
| SLC1A2, ILIR2, FAM20A, RETN, MMP8 | | | | | | | | | |
| IFI27, IFIT1, IFI44L, SIGLEC1, ISG15, | 0.864 | 0.854 | 0.873 | 0.930 | 0.882 | 0.979 | 0.953 | 0.902 | 1 |
| SLC1A2, IL1R2, OLAH, RETN, MMP8 | | | | | | | | | |
| OAS1, IFIT1, SIGLEC1, ISG15, SLC1A2, | 0.864 | 0.854 | 0.873 | 0.929 | 0.883 | 0.975 | 0.961 | 0.901 | 1 |
| IL1R2, OLAH, FAM20A, RETN, MMP8 | | | | | | | | | |
| RSAD2, IFI27, IFI44L, SIGLEC1, ISG15, | 0.863 | 0.854 | 0.873 | 0.929 | 0.88 | 0.978 | 0.945 | 0.891 | 0.998 |
| HERC6, SLC1A2, IL1R2, OLAH, MMP8 | | | | | | | | | |
| RSAD2, IFI27, IFI44L, ISG15, HERC6, | 0.863 | 0.854 | 0.873 | 0.928 | 0.878 | 0.979 | 0.941 | 0.881 | 1 |
| SLC1A2, IL1R2, OLAH, RETN, MMP8 | | | | | | | | | |
| OAS1, SIGLEC1, ISG15, HERC6, SLC1A2, | 0.863 | 0.853 | 0.874 | 0.924 | 0.876 | 0.973 | 0.959 | 0.897 | 1 |
| IL1R2, OLAH, FAM20A, RETN, MMP8 | | | | | | | | | |
| RSAD2, OAS1, IFIT1, SIGLEC1, HERC6, | 0.863 | 0.854 | 0.873 | 0.928 | 0.881 | 0.975 | 0.960 | 0.897 | 1 |
| SLC1A2, IL1R2, FAM20A, RETN, MMP8 | | | | | | | | | |
| IFI27, IFIT1, IFI44L, SIGLEC1, ISG15, | 0.863 | 0.854 | 0.873 | 0.928 | 0.879 | 0.977 | 0.943 | 0.890 | 0.997 |
| HERC6, SLC1A2, IL1R2, OLAH, MMP8 | | | | | | | | | |
| RSAD2, OAS1, IFIT1, IFI44L, SIGLEC1, | 0.863 | 0.854 | 0.873 | 0.920 | 0.864 | 0.977 | 0.954 | 0.900 | 1 |
| ISG15, HERC6, IL1R2, FAM20A, RETN | | | | | | | | | |
| IFI27, OAS1, IFIT1, IFI44L, ISG15, SLC1A2, | 0.863 | 0.853 | 0.874 | 0.931 | 0.882 | 0.979 | 0.927 | 0.844 | 1 |
| IL1R2, FAM20A, RETN, MMP8 | | | | | | | | | |
| RSAD2, OAS1, IFIT1, IFI44L, SIGLEC1, | 0.863 | 0.853 | 0.873 | 0.918 | 0.86 | 0.976 | 0.953 | 0.898 | 1 |
| HERC6, IL1R2, OLAH, FAM20A, RETN | | | | | | | | | |
| RSAD2, IFIT1, IFI44L, SIGLEC1, ISG15, | 0.863 | 0.853 | 0.873 | 0.920 | 0.863 | 0.978 | 0.967 | 0.916 | 1 |
| IL1R2, OLAH, FAM20A, RETN, MMP8 | | | | | | | | | |
| IFI27, IFIT1, IFI44L, SIGLEC1, ISG15, | 0.863 | 0.854 | 0.873 | 0.928 | 0.878 | 0.978 | 0.941 | 0.884 | 0.998 |
| HERC6, SLC1A2, IL1R2, OLAH, RETN | | | | | | | | | |
| IFI27, OAS1, IFIT1, IFI44L, SIGLEC1, | 0.863 | 0.853 | 0.874 | 0.929 | 0.877 | 0.980 | 0.950 | 0.892 | 1 |
| ISG15, SLC1A2, OLAH, RETN, MMP8 | | | | | | | | | |
| RSAD2, IFIT1, SIGLEC1, ISG15, HERC6, | 0.863 | 0.854 | 0.873 | 0.926 | 0.878 | 0.974 | 0.957 | 0.889 | 1 |
| SLC1A2, ILIR2, FAM20A, RETN, MMP8 | | | | | | | | | |
| RSAD2, IFI27, OAS1, IFIT1, ISG15, HERC6, | 0.863 | 0.853 | 0.873 | 0.929 | 0.880 | 0.977 | 0.933 | 0.866 | 0.999 |
| SLC1A2, IL1R2, OLAH, MMP8 | | | | | | | | | |
| RSAD2, IFI27, OAS1, IFIT1, IFI44L, | 0.863 | 0.854 | 0.873 | 0.927 | 0.873 | 0.980 | 0.958 | 0.901 | 1 |
| SIGLEC1, SLC1A2, OLAH, RETN, MMP8 | | | | | | | | | |
| RSAD2, IFI27, OAS1, IFIT1, SIGLEC1, | 0.863 | 0.854 | 0.873 | 0.930 | 0.880 | 0.981 | 0.947 | 0.885 | 1 |
| ISG15, SLC1A2, IL1R2, OLAH, RETN | | | | | | | | | |
| OAS1, IFIT1, IFI44L, SIGLEC1, ISG15, | 0.863 | 0.853 | 0.873 | 0.927 | 0.879 | 0.975 | 0.935 | 0.866 | 1 |
| HERC6, SLC1A2, IL1R2, OLAH, FAM20A | | | | | | | | | |
| IFI27, OAS1, IFI44L, SIGLEC1, ISG15, | 0.863 | 0.854 | 0.872 | 0.927 | 0.880 | 0.974 | 0.955 | 0.907 | 1 |
| HERC6, SLC1A2, IL1R2, RETN, MMP8 | | | | | | | | | |
| IFI27, OAS1, IFIT1, IFI44L, SIGLEC1, | 0.863 | 0.853 | 0.873 | 0.928 | 0.879 | 0.977 | 0.948 | 0.895 | 1 |
| ISG15, SLC1A2, IL1R2, OLAH, MMP8 | | | | | | | | | |
| RSAD2, IFI27, IFIT1, IFI44L, SIGLEC1, | 0.863 | 0.854 | 0.873 | 0.930 | 0.880 | 0.979 | 0.958 | 0.908 | 1 |
| SLC1A2, IL1R2, OLAH, RETN, MMP8 | | | | | | | | | |
| IFI27, OAS1, IFIT1, IFI44L, SIGLEC1, | 0.863 | 0.853 | 0.872 | 0.929 | 0.878 | 0.979 | 0.948 | 0.890 | 1 |
| ISG15, SLC1A2, IL1R2, OLAH, RETN | | | | | | | | | |
| RSAD2, OAS1, IFIT1, IFI44L, SIGLEC1, | 0.863 | 0.852 | 0.873 | 0.926 | 0.877 | 0.976 | 0.933 | 0.863 | 1 |
| ISG15, HERC6, SLC1A2, OLAH, FAM20A | | | | | | | | | |
| RSAD2, OAS1, IFI44L, SIGLEC1, HERC6, | 0.863 | 0.852 | 0.874 | 0.920 | 0.864 | 0.976 | 0.961 | 0.903 | 1 |
| IL1R2, OLAH, FAM20A, RETN, MMP8 | | | | | | | | | |
| RSAD2, IFI27, OAS1, SIGLEC1, ISG15, | 0.863 | 0.854 | 0.872 | 0.926 | 0.878 | 0.975 | 0.958 | 0.906 | 1 |
| HERC6, SLC1A2, IL1R2, RETN, MMP8 | | | | | | | | | |
| RSAD2, IFI27, IFIT1, IFI44L, SIGLEC1, | 0.863 | 0.854 | 0.872 | 0.928 | 0.879 | 0.976 | 0.948 | 0.897 | 0.999 |
| HERC6, SLC1A2, ILIR2, OLAH, MMP8 | | | | | | | | | |
| RSAD2, IFI27, OAS1, IFIT1, IFI44L, ISG15, | 0.863 | 0.852 | 0.873 | 0.930 | 0.884 | 0.976 | 0.926 | 0.840 | 1 |
| HERC6, SLC1A2, IL1R2, FAM20A | | | | | | | | | |
| RSAD2, OAS1, SIGLEC1, HERC6, SLC1A2, | 0.863 | 0.852 | 0.873 | 0.927 | 0.879 | 0.975 | 0.959 | 0.897 | 1 |
| IL1R2, OLAH, FAM20A, RETN, MMP8 | | | | | | | | | |
| RSAD2, OAS1, IFI44L, SIGLEC1, ISG15, | 0.863 | 0.853 | 0.873 | 0.918 | 0.861 | 0.976 | 0.959 | 0.897 | 1 |
| HERC6, IL1R2, FAM20A, RETN, MMP8 | | | | | | | | | |
| RSAD2, OAS1, IFIT1, IFI44L, SIGLEC1, | 0.863 | 0.853 | 0.873 | 0.926 | 0.878 | 0.974 | 0.938 | 0.871 | 1 |
| HERC6, SLC1A2, IL1R2, OLAH, FAM20A | | | | | | | | | |
| RSAD2, IFI27, OAS1, IFIT1, IFI44L, | 0.863 | 0.854 | 0.872 | 0.929 | 0.877 | 0.981 | 0.948 | 0.885 | 1 |
| SIGLEC1, ISG15, SLC1A2, OLAH, RETN | | | | | | | | | |
| RSAD2, IFI27, OAS1, IFI44L, SIGLEC1, | 0.863 | 0.853 | 0.872 | 0.929 | 0.881 | 0.977 | 0.950 | 0.892 | 1 |
| HERC6, SLC1A2, IL1R2, RETN, MMP8 | | | | | | | | | |
| RSAD2, IFI27, IFI44L, ISG15, HERC6, | 0.863 | 0.852 | 0.874 | 0.933 | 0.884 | 0.981 | 0.926 | 0.841 | 1 |
| SLC1A2, IL1R2, FAM20A, RETN, MMP8 | | | | | | | | | |
| RSAD2, IFI27, IFIT1, IFI44L, ISG15, | 0.863 | 0.853 | 0.873 | 0.928 | 0.876 | 0.979 | 0.941 | 0.882 | 1 |
| HERC6, SLC1A2, OLAH, RETN, MMP8 | | | | | | | | | |
| RSAD2, IFI27, OAS1, IFIT1, ISG15, HERC6, | 0.863 | 0.852 | 0.873 | 0.930 | 0.883 | 0.977 | 0.927 | 0.842 | 1 |
| SLC1A2, IL1R2, FAM20A, MMP8 | | | | | | | | | |
| RSAD2, IFI27, IFIT1, IFI44L, SIGLEC1, | 0.863 | 0.853 | 0.872 | 0.930 | 0.880 | 0.980 | 0.942 | 0.886 | 0.999 |
| ISG15, HERC6, SLC1A2, OLAH, MMP8 | | | | | | | | | |
| RSAD2, OAS1, IFIT1, IFI44L, SIGLEC1, | 0.863 | 0.852 | 0.873 | 0.922 | 0.865 | 0.979 | 0.967 | 0.914 | 1 |
| ISG15, IL1R2, FAM20A, RETN, MMP8 | | | | | | | | | |
| RSAD2, IFI27, IFIT1, IFI44L, SIGLEC1, | 0.863 | 0.854 | 0.872 | 0.928 | 0.879 | 0.978 | 0.943 | 0.890 | 0.997 |
| ISG15, SLC1A2, IL1R2, OLAH, MMP8 | | | | | | | | | |
| RSAD2, SIGLEC1, ISG15, HERC6, SLC1A2, | 0.863 | 0.853 | 0.872 | 0.927 | 0.879 | 0.974 | 0.958 | 0.893 | 1 |
| IL1R2, OLAH, FAM20A, RETN, MMP8 | | | | | | | | | |
| RSAD2, IFI27, IFIT1, IFI44L, SIGLEC1, | 0.863 | 0.853 | 0.872 | 0.929 | 0.880 | 0.978 | 0.942 | 0.888 | 0.997 |
| ISG15, HERC6, SLC1A2, IL1R2, OLAH | | | | | | | | | |
| RSAD2, IFI27, OAS1, IFIT1, IFI44L, ISG15, | 0.863 | 0.853 | 0.873 | 0.928 | 0.877 | 0.979 | 0.933 | 0.861 | 1 |
| HERC6, SLC1A2, OLAH, RETN | | | | | | | | | |
| RSAD2, OAS1, IFIT1, SIGLEC1, ISG15, | 0.863 | 0.853 | 0.873 | 0.926 | 0.877 | 0.974 | 0.948 | 0.884 | 1 |
| HERC6, SLC1A2, IL1R2, OLAH, FAM20A | | | | | | | | | |
| RSAD2, IFI27, OAS1, IFIT1, ISG15, HERC6, | 0.863 | 0.853 | 0.872 | 0.932 | 0.884 | 0.980 | 0.925 | 0.838 | 1 |
| SLC1A2, IL1R2, FAM20A, RETN | | | | | | | | | |
| OAS1, IFIT1, IFI44L, SIGLEC1, ISG15, | 0.863 | 0.853 | 0.873 | 0.926 | 0.876 | 0.975 | 0.929 | 0.859 | 1 |
| HERC6, SLC1A2, OLAH, FAM20A, RETN | | | | | | | | | |
| OAS1, IFIT1, IFI44L, SIGLEC1, ISG15, | 0.863 | 0.852 | 0.873 | 0.922 | 0.869 | 0.975 | 0.949 | 0.888 | 1 |
| SLC1A2, OLAH, FAM20A, RETN, MMP8 | | | | | | | | | |
| IFI27, OAS1, IFIT1, IFI44L, ISG15, HERC6, | 0.862 | 0.852 | 0.873 | 0.923 | 0.867 | 0.979 | 0.932 | 0.850 | 1 |
| SLC1A2, FAM20A, RETN, MMP8 | | | | | | | | | |
| IFI27, IFIT1, IFI44L, ISG15, HERC6, | 0.862 | 0.853 | 0.872 | 0.927 | 0.875 | 0.978 | 0.936 | 0.873 | 0.999 |
| SLC1A2, IL1R2, OLAH, RETN, MMP8 | | | | | | | | | |
| RSAD2, IFIT1, IFI44L, SIGLEC1, ISG15, | 0.862 | 0.852 | 0.872 | 0.925 | 0.876 | 0.975 | 0.937 | 0.867 | 1 |
| HERC6, SLC1A2, IL1R2, OLAH, FAM20A | | | | | | | | | |
| RSAD2, IFI27, OAS1, IFI44L, SIGLEC1, | 0.862 | 0.852 | 0.872 | 0.927 | 0.875 | 0.978 | 0.954 | 0.897 | 1 |
| SLC1A2, IL1R2, OLAH, RETN, MMP8 | | | | | | | | | |
| IFI27, OAS1, IFI44L, SIGLEC1, ISG15, | 0.862 | 0.852 | 0.872 | 0.930 | 0.881 | 0.979 | 0.952 | 0.898 | 1 |
| SLC1A2, IL1R2, OLAH, RETN, MMP8 | | | | | | | | | |
| RSAD2, IFI27, OAS1, IFIT1, IFI44L, | 0.862 | 0.852 | 0.872 | 0.932 | 0.884 | 0.979 | 0.925 | 0.838 | 1 |
| HERC6, SLC1A2, IL1R2, FAM20A, RETN | | | | | | | | | |
| RSAD2, IFI27, IFIT1, IFI44L, ISG15, | 0.862 | 0.852 | 0.873 | 0.930 | 0.881 | 0.980 | 0.928 | 0.845 | 1 |
| SLC1A2, IL1R2, FAM20A, RETN, MMP8 | | | | | | | | | |
| RSAD2, OAS1, SIGLEC1, ISG15, HERC6, | 0.862 | 0.852 | 0.872 | 0.926 | 0.878 | 0.974 | 0.958 | 0.892 | 1 |
| SLC1A2, IL1R2, FAM20A, RETN, MMP8 | | | | | | | | | |
| RSAD2, OAS1, IFIT1, IFI44L, SIGLEC1, | 0.862 | 0.852 | 0.872 | 0.916 | 0.857 | 0.976 | 0.940 | 0.880 | 1 |
| ISG15, HERC6, OLAH, FAM20A, RETN | | | | | | | | | |
| RSAD2, IFI27, OAS1, SIGLEC1, ISG15, | 0.862 | 0.852 | 0.873 | 0.930 | 0.880 | 0.980 | 0.951 | 0.892 | 1 |
| SLC1A2, IL1R2, OLAH, RETN, MMP8 | | | | | | | | | |
| OAS1, IFIT1, IFI44L, SIGLEC1, ISG15, | 0.862 | 0.852 | 0.872 | 0.928 | 0.881 | 0.974 | 0.959 | 0.893 | 1 |
| SLC1A2, IL1R2, FAM20A, RETN, MMP8 | | | | | | | | | |
| RSAD2, IFIT1, IFI44L, SIGLEC1, ISG15, | 0.862 | 0.853 | 0.872 | 0.918 | 0.860 | 0.976 | 0.951 | 0.894 | 1 |
| HERC6, IL1R2, OLAH, FAM20A, RETN | | | | | | | | | |
| RSAD2, IFI27, IFIT1, IFI44L, SIGLEC1, | 0.862 | 0.853 | 0.871 | 0.929 | 0.879 | 0.978 | 0.943 | 0.884 | 1 |
| ISG15, SLC1A2, IL1R2, OLAH, RETN | | | | | | | | | |
| OAS1, IFIT1, IFI44L, SIGLEC1, SLC1A2, | 0.862 | 0.852 | 0.872 | 0.927 | 0.879 | 0.975 | 0.959 | 0.904 | 1 |
| IL1R2, OLAH, FAM20A, RETN, MMP8 | | | | | | | | | |
| RSAD2, IFI44L, SIGLEC1, ISG15, HERC6, | 0.862 | 0.851 | 0.873 | 0.922 | 0.872 | 0.971 | 0.959 | 0.897 | 1 |
| SLC1A2, ILIR2, OLAH, FAM20A, MMP8 | | | | | | | | | |
| IFIT1, IFI44L, SIGLEC1, ISG15, HERC6, | 0.862 | 0.852 | 0.872 | 0.926 | 0.876 | 0.975 | 0.938 | 0.868 | 1 |
| SLC1A2, IL1R2, OLAH, FAM20A, RETN | | | | | | | | | |
| RSAD2, IFI27, OAS1, IFIT1, IFI44L, | 0.862 | 0.852 | 0.873 | 0.930 | 0.881 | 0.980 | 0.927 | 0.844 | 1 |
| SLC1A2, IL1R2, FAM20A, RETN, MMP8 | | | | | | | | | |
| IFI44L, SIGLEC1, ISG15, HERC6, SLC1A2, | 0.862 | 0.852 | 0.872 | 0.923 | 0.873 | 0.972 | 0.957 | 0.890 | 1 |
| IL1R2, OLAH, FAM20A, RETN, MMP8 | | | | | | | | | |
| RSAD2, IFI27, IFIT1, IFI44L, ISG 15, | 0.862 | 0.852 | 0.872 | 0.930 | 0.882 | 0.979 | 0.926 | 0.842 | 1 |
| HERC6, SLC1A2, IL1R2, FAM20A, RETN | | | | | | | | | |
| RSAD2, IFI27, IFIT1, IFI44L, ISG15, | 0.862 | 0.852 | 0.872 | 0.930 | 0.883 | 0.977 | 0.927 | 0.841 | 1 |
| HERC6, SLC1A2, ILIR2, FAM20A, MMP8 | | | | | | | | | |
| RSAD2, OAS1, IFIT1, SIGLEC1, SLC1A2, | 0.862 | 0.852 | 0.872 | 0.926 | 0.878 | 0.975 | 0.961 | 0.903 | 1 |
| IL1R2, OLAH, FAM20A, RETN, MMP8 | | | | | | | | | |
| RSAD2, IFI27, IFI44L, SIGLEC1, ISG15, | 0.862 | 0.852 | 0.872 | 0.931 | 0.883 | 0.979 | 0.954 | 0.903 | 1 |
| SLC1A2, IL1R2, OLAH, RETN, MMP8 | | | | | | | | | |
| RSAD2, IFI44L, SIGLEC1, ISG15, HERC6, | 0.862 | 0.852 | 0.872 | 0.918 | 0.860 | 0.975 | 0.961 | 0.903 | 1 |
| IL1R2, OLAH, FAM20A, RETN, MMP8 | | | | | | | | | |
| RSAD2, IFI27, OAS1, IFIT1, IFI44L, | 0.862 | 0.853 | 0.871 | 0.922 | 0.874 | 0.971 | 0.948 | 0.900 | 0.996 |
| SIGLEC1, ISG15, HERC6, IL1R2, MMP8 | | | | | | | | | |
| RSAD2, IFI27, OAS1, IFIT1, IFI44L, ISG15, | 0.862 | 0.851 | 0.872 | 0.924 | 0.869 | 0.979 | 0.929 | 0.848 | 1 |
| HERC6, SLC1A2, FAM20A, RETN | | | | | | | | | |
| OAS1, IFIT1, IFI44L, SIGLEC1, ISG15, | 0.862 | 0.852 | 0.872 | 0.925 | 0.875 | 0.976 | 0.939 | 0.868 | 1 |
| HERC6, SLC1A2, FAM20A, RETN, MMP8 | | | | | | | | | |
| RSAD2, IFI27, OAS1, IFIT1, IFI44L, | 0.862 | 0.852 | 0.872 | 0.926 | 0.877 | 0.975 | 0.937 | 0.873 | 1 |
| HERC6, SLC1A2, IL1R2, OLAH, MMP8 | | | | | | | | | |
| RSAD2, IFI27, OAS1, IFI44L, ISG15, | 0.862 | 0.851 | 0.873 | 0.933 | 0.886 | 0.980 | 0.926 | 0.840 | 1 |
| HERC6, SLC1A2, IL1R2, FAM20A, RETN | | | | | | | | | |
| RSAD2, IFI27, OAS1, IFI44L, SIGLEC1, | 0.862 | 0.852 | 0.871 | 0.923 | 0.868 | 0.978 | 0.942 | 0.877 | 1 |
| ISG15, HERC6, SLC1A2, RETN, MMP8 | | | | | | | | | |
| RSAD2, IFI27, OAS1, IFIT1, IFI44L, ISG15, | 0.862 | 0.852 | 0.872 | 0.927 | 0.877 | 0.978 | 0.929 | 0.861 | 0.998 |
| HERC6, SLC1A2, OLAH, MMP8 | | | | | | | | | |
| RSAD2, IFI27, OAS1, IFIT1, IFI44L, ISG15, | 0.862 | 0.851 | 0.872 | 0.927 | 0.879 | 0.975 | 0.926 | 0.840 | 1 |
| SLC1A2, IL1R2, FAM20A, MMP8 | | | | | | | | | |
| RSAD2, IFI27, OAS1, IFIT1, IFI44L, | 0.862 | 0.852 | 0.871 | 0.921 | 0.866 | 0.975 | 0.961 | 0.915 | 1 |
| SIGLEC1, ISG15, IL1R2, RETN, MMP8 | | | | | | | | | |
| IFI27, OAS1, IFIT1, IFI44L, ISG15, HERC6, | 0.862 | 0.851 | 0.872 | 0.927 | 0.876 | 0.978 | 0.932 | 0.864 | 0.999 |
| SLC1A2, IL1R2, OLAH, RETN | | | | | | | | | |
| RSAD2, IFI27, OAS1, IFIT1, ISG15, | 0.862 | 0.852 | 0.872 | 0.928 | 0.877 | 0.979 | 0.941 | 0.878 | 1 |
| SLC1A2, IL1R2, OLAH, RETN, MMP8 | | | | | | | | | |
| RSAD2, IFIT1, IFI44L, SIGLEC1, ISG15, | 0.862 | 0.852 | 0.871 | 0.922 | 0.869 | 0.975 | 0.946 | 0.884 | 1 |
| SLC1A2, OLAH, FAM20A, RETN, MMP8 | | | | | | | | | |
| RSAD2, OAS1, IFI44L, SIGLEC1, ISG15, | 0.862 | 0.851 | 0.872 | 0.922 | 0.872 | 0.972 | 0.960 | 0.900 | 1 |
| SLC1A2, IL1R2, OLAH, FAM20A, MMP8 | | | | | | | | | |
| RSAD2, OAS1, SIGLEC1, ISG15, SLC1A2, | 0.861 | 0.851 | 0.872 | 0.923 | 0.874 | 0.972 | 0.960 | 0.900 | 1 |
| ILIR2, OLAH, FAM20A, RETN, MMP8 | | | | | | | | | |
| RSAD2, IFI27, OAS1, IFIT1, IFI44L, | 0.861 | 0.851 | 0.872 | 0.921 | 0.863 | 0.978 | 0.939 | 0.866 | 1 |
| SIGLEC1, ISG15, SLC1A2, FAM20A, | | | | | | | | | |
| MMP8 | | | | | | | | | |
| RSAD2, IFI27, OAS1, IFIT1, IFI44L, ISG15, | 0.861 | 0.852 | 0.871 | 0.928 | 0.880 | 0.977 | 0.933 | 0.867 | 0.998 |
| HERC6, SLC1A2, IL1R2, OLAH | | | | | | | | | |
| RSAD2, IFIT1, SIGLEC1, ISG15, SLC1A2, | 0.861 | 0.852 | 0.871 | 0.928 | 0.881 | 0.975 | 0.964 | 0.907 | 1 |
| IL1R2, OLAH, FAM20A, RETN, MMP8 | | | | | | | | | |
| RSAD2, IFI27, IFIT1, IFI44L, SIGLEC1, | 0.861 | 0.852 | 0.870 | 0.926 | 0.878 | 0.975 | 0.957 | 0.911 | 1 |
| HERC6, SLC1A2, IL1R2, RETN, MMP8 | | | | | | | | | |
| RSAD2, OAS1, IFIT1, IFI44L, SIGLEC1, | 0.861 | 0.851 | 0.871 | 0.926 | 0.88 | 0.973 | 0.961 | 0.897 | 1 |
| ISG15, SLC1A2, IL1R2, FAM20A, MMP8 | | | | | | | | | |
| RSAD2, OAS1, IFI44L, SIGLEC1, ISG15, | 0.861 | 0.851 | 0.872 | 0.916 | 0.858 | 0.975 | 0.963 | 0.908 | 1 |
| ILIR2, OLAH, FAM20A, RETN, MMP8 | | | | | | | | | |
| RSAD2, IFI27, OAS1, IFI44L, ISG15, | 0.861 | 0.851 | 0.871 | 0.930 | 0.881 | 0.978 | 0.930 | 0.863 | 0.998 |
| HERC6, SLC1A2, IL1R2, OLAH, MMP8 | | | | | | | | | |
| RSAD2, OAS1, IFIT1, IFI44L, SIGLEC1, | 0.861 | 0.851 | 0.871 | 0.926 | 0.876 | 0.976 | 0.933 | 0.863 | 1 |
| HERC6, SLC1A2, OLAH, FAM20A, RETN | | | | | | | | | |
| OAS1, IFIT1, SIGLEC1, ISG15, HERC6, | 0.861 | 0.851 | 0.871 | 0.924 | 0.875 | 0.973 | 0.942 | 0.872 | 1 |
| SLC1A2, ILIR2, OLAH, FAM20A, RETN | | | | | | | | | |
| RSAD2, IFI27, OAS1, IFIT1, SIGLEC1, | 0.861 | 0.850 | 0.872 | 0.929 | 0.876 | 0.981 | 0.952 | 0.893 | 1 |
| ISG15, SLC1A2, OLAH, RETN, MMP8 | | | | | | | | | |
| RSAD2, OAS1, SIGLEC1, ISG15, HERC6, | 0.861 | 0.851 | 0.871 | 0.920 | 0.867 | 0.974 | 0.947 | 0.879 | 1 |
| SLC1A2, OLAH, FAM20A, RETN, MMP8 | | | | | | | | | |
| OAS1, IFIT1, IFI44L, SIGLEC1, HERC6, | 0.861 | 0.851 | 0.871 | 0.927 | 0.879 | 0.975 | 0.934 | 0.865 | 1 |
| SLC1A2, ILIR2, OLAH, FAM20A, RETN | | | | | | | | | |
| OAS1, IFI44L, SIGLEC1, ISG15, HERC6, | 0.861 | 0.851 | 0.871 | 0.926 | 0.878 | 0.974 | 0.959 | 0.895 | 1 |
| SLC1A2, IL1R2, FAM20A, RETN, MMP8 | | | | | | | | | |
| RSAD2, IFI27, OAS1, IFIT1, ISG15, | 0.861 | 0.851 | 0.871 | 0.931 | 0.883 | 0.980 | 0.927 | 0.844 | 1 |
| SLC1A2, IL1R2, FAM20A, RETN, MMP8 | | | | | | | | | |
| RSAD2, IFI27, OAS1, IFI44L, SIGLEC1, | 0.861 | 0.851 | 0.871 | 0.929 | 0.877 | 0.981 | 0.954 | 0.896 | 1 |
| ISG15, SLC1A2, OLAH, RETN, MMP8 | | | | | | | | | |
| RSAD2, OAS1, IFIT1, IFI44L, SIGLEC1, | 0.861 | 0.851 | 0.871 | 0.926 | 0.878 | 0.973 | 0.946 | 0.879 | 1 |
| ISG15, HERC6, SLC1A2, IL1R2, FAM20A | | | | | | | | | |
| RSAD2, IFI27, OAS1, IFI44L, SIGLEC1, | 0.861 | 0.849 | 0.872 | 0.924 | 0.868 | 0.981 | 0.945 | 0.871 | 1 |
| ISG15, SLC1A2, FAM20A, RETN, MMP8 | | | | | | | | | |
| RSAD2, IFI27, IFIT1, IFI44L, ISG15, | 0.861 | 0.851 | 0.870 | 0.926 | 0.875 | 0.976 | 0.934 | 0.869 | 0.999 |
| HERC6, SLC1A2, IL1R2, OLAH, RETN | | | | | | | | | |
| RSAD2, IFI27, IFIT1, IFI44L, ISG15, | 0.861 | 0.851 | 0.870 | 0.926 | 0.874 | 0.978 | 0.943 | 0.885 | 1 |
| SLC1A2, IL1R2, OLAH, RETN, MMP8 | | | | | | | | | |
| RSAD2, IFIT1, IFI44L, SIGLEC1, SLC1A2, | 0.861 | 0.85 | 0.871 | 0.927 | 0.879 | 0.975 | 0.959 | 0.902 | 1 |
| IL1R2, OLAH, FAM20A, RETN, MMP8 | | | | | | | | | |
| RSAD2, IFIT1, SIGLEC1, ISG15, HERC6, | 0.861 | 0.851 | 0.871 | 0.924 | 0.875 | 0.974 | 0.943 | 0.872 | 1 |
| SLC1A2, IL1R2, OLAH, FAM20A, RETN | | | | | | | | | |
| RSAD2, IFIT1, IFI44L, SIGLEC1, ISG15, | 0.861 | 0.85 | 0.871 | 0.925 | 0.876 | 0.975 | 0.928 | 0.855 | 1 |
| HERC6, SLC1A2, OLAH, FAM20A, RETN | | | | | | | | | |
| RSAD2, IFI27, IFIT1, IFI44L, SIGLEC1, | 0.861 | 0.850 | 0.871 | 0.930 | 0.879 | 0.980 | 0.952 | 0.897 | 1 |
| ISG15, SLC1A2, OLAH, RETN, MMP8 | | | | | | | | | |
| RSAD2, IFI27, OAS1, IFIT1, IFI44L, ISG15, | 0.861 | 0.851 | 0.870 | 0.926 | 0.877 | 0.976 | 0.934 | 0.869 | 0.998 |
| SLC1A2, IL1R2, OLAH, MMP8 | | | | | | | | | |
| IFI27, OAS1, IFIT1, IFI44L, ISG15, HERC6, | 0.861 | 0.850 | 0.871 | 0.927 | 0.878 | 0.976 | 0.933 | 0.869 | 0.996 |
| SLC1A2, IL1R2, OLAH, MMP8 | | | | | | | | | |
| RSAD2, OAS1, IFI44L, SIGLEC1, ISG15, | 0.861 | 0.850 | 0.871 | 0.924 | 0.876 | 0.972 | 0.959 | 0.893 | 1 |
| HERC6, SLC1A2, IL1R2, FAM20A, MMP8 | | | | | | | | | |
| RSAD2, IFI27, OAS1, IFIT1, IFI44L, | 0.861 | 0.851 | 0.871 | 0.926 | 0.873 | 0.978 | 0.942 | 0.881 | 1 |
| SLC1A2, IL1R2, OLAH, RETN, MMP8 | | | | | | | | | |
| OAS1, IFI44L, SIGLEC1, HERC6, SLC1A2, | 0.860 | 0.850 | 0.871 | 0.925 | 0.876 | 0.974 | 0.958 | 0.896 | 1 |
| IL1R2, OLAH, FAM20A, RETN, MMP8 | | | | | | | | | |
| IFI27, IFIT1, IFI44L, SIGLEC1, ISG15, | 0.860 | 0.851 | 0.870 | 0.926 | 0.878 | 0.974 | 0.954 | 0.908 | 1 |
| HERC6, SLC1A2, IL1R2, RETN, MMP8 | | | | | | | | | |
| RSAD2, OAS1, IFIT1, SIGLEC1, HERC6, | 0.860 | 0.850 | 0.870 | 0.926 | 0.877 | 0.974 | 0.947 | 0.882 | 1 |
| SLC1A2, IL1R2, OLAH, FAM20A, RETN | | | | | | | | | |
| RSAD2, IFIT1, IFI44L, SIGLEC1, ISG15, | 0.860 | 0.851 | 0.870 | 0.925 | 0.874 | 0.976 | 0.939 | 0.866 | 1 |
| HERC6, SLC1A2, FAM20A, RETN, MMP8 | | | | | | | | | |
| IFI27, OAS1, IFIT1, IFI44L, SIGLEC1, | 0.860 | 0.851 | 0.870 | 0.926 | 0.879 | 0.974 | 0.958 | 0.909 | 1 |
| ISG15, SLC1A2, IL1R2, RETN, MMP8 | | | | | | | | | |
| RSAD2, IFIT1, IFI44L, SIGLEC1, HERC6, | 0.860 | 0.850 | 0.870 | 0.926 | 0.877 | 0.974 | 0.937 | 0.867 | 1 |
| SLC1A2, IL1R2, OLAH, FAM20A, RETN | | | | | | | | | |
| OAS1, IFI44L, SIGLEC1, ISG15, SLC1A2, | 0.860 | 0.850 | 0.870 | 0.922 | 0.872 | 0.972 | 0.960 | 0.900 | 1 |
| ILIR2, OLAH, FAM20A, RETN, MMP8 | | | | | | | | | |
| RSAD2, IFIT1, IFI44L, SIGLEC1, ISG15, | 0.860 | 0.851 | 0.870 | 0.927 | 0.880 | 0.974 | 0.959 | 0.893 | 1 |
| SLC1A2, IL1R2, FAM20A, RETN, MMP8 | | | | | | | | | |
| OAS1, IFIT1, IFI44L, SIGLEC1, ISG15, | 0.860 | 0.850 | 0.870 | 0.926 | 0.879 | 0.973 | 0.941 | 0.872 | 1 |
| HERC6, SLC1A2, IL1R2, FAM20A, RETN | | | | | | | | | |
| RSAD2, OAS1, IFIT1, IFI44L, SIGLEC1, | 0.860 | 0.850 | 0.870 | 0.911 | 0.849 | 0.973 | 0.963 | 0.913 | 1 |
| ISG15, IL1R2, OLAH, FAM20A, RETN | | | | | | | | | |
| RSAD2, OAS1, IFIT1, SIGLEC1, ISG15, | 0.860 | 0.850 | 0.870 | 0.924 | 0.874 | 0.974 | 0.941 | 0.875 | 1 |
| HERC6, SLC1A2, OLAH, FAM20A, RETN | | | | | | | | | |
| RSAD2, IFI27, IFIT1, SIGLEC1, ISG15, | 0.860 | 0.851 | 0.869 | 0.926 | 0.878 | 0.973 | 0.962 | 0.921 | 1 |
| HERC6, SLC1A2, IL1R2, RETN, MMP8 | | | | | | | | | |
| IFI27, OAS1, IFIT1, IFI44L, ISG15, SLC1A2, | 0.860 | 0.850 | 0.870 | 0.926 | 0.874 | 0.978 | 0.941 | 0.879 | 1 |
| ILIR2, OLAH, RETN, MMP8 | | | | | | | | | |
| RSAD2, OAS1, IFIT1, IFI44L, SIGLEC1, | 0.860 | 0.849 | 0.871 | 0.925 | 0.873 | 0.977 | 0.948 | 0.888 | 1 |
| SLC1A2, OLAH, FAM20A, RETN, MMP8 | | | | | | | | | |
| RSAD2, OAS1, IFIT1, IFI44L, SIGLEC1, | 0.860 | 0.850 | 0.870 | 0.924 | 0.872 | 0.975 | 0.939 | 0.867 | 1 |
| HERC6, SLC1A2, FAM20A, RETN, MMP8 | | | | | | | | | |
| RSAD2, IFI27, OAS1, IFIT1, IFI44L, | 0.860 | 0.849 | 0.871 | 0.922 | 0.866 | 0.979 | 0.936 | 0.854 | 1 |
| HERC6, SLC1A2, FAM20A, RETN, MMP8 | | | | | | | | | |
| RSAD2, OASI, IFIT1, SIGLEC1, ISG15, | 0.860 | 0.850 | 0.869 | 0.928 | 0.881 | 0.975 | 0.959 | 0.893 | 1 |
| SLC1A2, IL1R2, FAM20A, RETN, MMP8 | | | | | | | | | |
| RSAD2, IFI27, IFIT1, IFI44L, ISG15, | 0.860 | 0.850 | 0.869 | 0.929 | 0.880 | 0.977 | 0.932 | 0.868 | 0.995 |
| HERC6, SLC1A2, IL1R2, OLAH, MMP8 | | | | | | | | | |
| RSAD2, IFI27, OAS1, IFI44L, ISG15, | 0.860 | 0.849 | 0.871 | 0.930 | 0.882 | 0.978 | 0.923 | 0.835 | 1 |
| SLC1A2, IL1R2, FAM20A, RETN, MMP8 | | | | | | | | | |
| RSAD2, OAS1, IFIT1, SIGLEC1, ISG15, | 0.860 | 0.850 | 0.870 | 0.922 | 0.869 | 0.975 | 0.943 | 0.873 | 1 |
| HERC6, SLC1A2, FAM20A, RETN, MMP8 | | | | | | | | | |
| RSAD2, IFI27, OAS1, IFI44L, ISG15, | 0.859 | 0.849 | 0.870 | 0.929 | 0.877 | 0.980 | 0.939 | 0.874 | 1 |
| SLC1A2, IL1R2, OLAH, RETN, MMP8 | | | | | | | | | |
| RSAD2, IFI44L, SIGLEC1, HERC6, | 0.859 | 0.849 | 0.870 | 0.926 | 0.878 | 0.974 | 0.959 | 0.898 | 1 |
| SLC1A2, IL1R2, OLAH, FAM20A, RETN, | | | | | | | | | |
| MMP8 | | | | | | | | | |
| RSAD2, IFI27, IFIT1, IFI44L, ISG 15, | 0.859 | 0.849 | 0.870 | 0.927 | 0.872 | 0.981 | 0.936 | 0.857 | 1 |
| HERC6, SLC1A2, FAM20A, RETN, MMP8 | | | | | | | | | |
| RSAD2, IFI27, OAS1, IFIT1, IFI44L, ISG15, | 0.859 | 0.850 | 0.868 | 0.922 | 0.871 | 0.974 | 0.932 | 0.859 | 1 |
| HERC6, SLC1A2, IL1R2, RETN | | | | | | | | | |
| RSAD2, IFI27, OAS1, IFIT1, IFI44L, ISG15, | 0.859 | 0.849 | 0.869 | 0.928 | 0.877 | 0.979 | 0.935 | 0.865 | 1 |
| SLC1A2, IL1R2, OLAH, RETN | | | | | | | | | |
| RSAD2, OAS1, IFI44L, SIGLEC1, ISG15, | 0.859 | 0.849 | 0.870 | 0.921 | 0.869 | 0.973 | 0.943 | 0.876 | 1 |
| HERC6, SLC1A2, OLAH, FAM20A, MMP8 | | | | | | | | | |
| RSAD2, IFI27, OAS1, IFIT1, IFI44L, ISG15, | 0.859 | 0.849 | 0.869 | 0.924 | 0.869 | 0.979 | 0.941 | 0.877 | 1 |
| SLC1A2, OLAH, RETN, MMP8 | | | | | | | | | |
| RSAD2, OAS1, IFIT1, IFI44L, SIGLEC1, | 0.859 | 0.849 | 0.869 | 0.927 | 0.881 | 0.974 | 0.963 | 0.906 | 1 |
| SLC1A2, ILIR2, FAM20A, RETN, MMP8 | | | | | | | | | |
| RSAD2, IFI27, OAS1, IFIT1, IFI44L, | 0.859 | 0.850 | 0.869 | 0.923 | 0.872 | 0.974 | 0.935 | 0.866 | 1 |
| HERC6, SLC1A2, IL1R2, RETN, MMP8 | | | | | | | | | |
| RSAD2, IFI44L, SIGLEC1, ISG15, SLC1A2, | 0.859 | 0.849 | 0.869 | 0.921 | 0.871 | 0.97 | 0.959 | 0.897 | 1 |
| IL1R2, OLAH, FAM20A, RETN, MMP8 | | | | | | | | | |
| RSAD2, IFI27, OAS1, IFIT1, ISG15, HERC6, | 0.859 | 0.848 | 0.870 | 0.922 | 0.866 | 0.979 | 0.933 | 0.851 | 1 |
| SLC1A2, FAM20A, RETN, MMP8 | | | | | | | | | |
| RSAD2, OAS1, IFIT1, IFI44L, SIGLEC1, | 0.859 | 0.848 | 0.870 | 0.913 | 0.849 | 0.978 | 0.959 | 0.910 | 1 |
| ISG15, OLAH, FAM20A, RETN, MMP8 | | | | | | | | | |
| RSAD2, IFI27, OAS1, IFI44L, ISG15, | 0.859 | 0.847 | 0.871 | 0.925 | 0.869 | 0.98 | 0.935 | 0.854 | 1 |
| HERC6, SLC1A2, FAM20A, RETN, MMP8 | | | | | | | | | |
| RSAD2, OAS1, IFI44L, SIGLEC1, HERC6, | 0.859 | 0.849 | 0.869 | 0.926 | 0.878 | 0.974 | 0.957 | 0.891 | 1 |
| SLC1A2, IL1R2, FAM20A, RETN, MMP8 | | | | | | | | | |
| OAS1, IFI44L, SIGLEC1, ISG15, HERC6, | 0.859 | 0.849 | 0.869 | 0.918 | 0.864 | 0.972 | 0.946 | 0.880 | 1 |
| SLC1A2, OLAH, FAM20A, RETN, MMP8 | | | | | | | | | |
| RSAD2, IFI27, OAS1, IFIT1, IFI44L, | 0.859 | 0.850 | 0.868 | 0.926 | 0.877 | 0.975 | 0.949 | 0.888 | 1 |
| SIGLEC1, ISG15, SLC1A2, IL1R2, RETN | | | | | | | | | |
| RSAD2, IFI44L, SIGLEC1, ISG15, HERC6, | 0.859 | 0.849 | 0.869 | 0.925 | 0.877 | 0.973 | 0.955 | 0.887 | 1 |
| SLC1A2, ILIR2, FAM20A, RETN, MMP8 | | | | | | | | | |
| RSAD2, OAS1, IFIT1, SIGLEC1, ISG15, | 0.859 | 0.849 | 0.869 | 0.925 | 0.877 | 0.973 | 0.946 | 0.879 | 1 |
| HERC6, SLC1A2, IL1R2, FAM20A, RETN | | | | | | | | | |
| RSAD2, IFI27, OAS1, IFIT1, IFI44L, | 0.859 | 0.850 | 0.868 | 0.925 | 0.876 | 0.974 | 0.957 | 0.902 | 1 |
| SIGLEC1, SLC1A2, IL1R2, RETN, MMP8 | | | | | | | | | |
| RSAD2, IFI27, OAS1, IFIT1, ISG15, HERC6, | 0.859 | 0.850 | 0.868 | 0.925 | 0.875 | 0.975 | 0.940 | 0.876 | 1 |
| SLC1A2, IL1R2, RETN, MMP8 | | | | | | | | | |
| RSAD2, IFI27, IFIT1, IFI44L, SIGLEC1, | 0.859 | 0.850 | 0.867 | 0.928 | 0.880 | 0.976 | 0.946 | 0.894 | 0.997 |
| ISG15, HERC6, SLC1A2, IL1R2, RETN | | | | | | | | | |
| RSAD2, IFI27, OAS1, IFI44L, SIGLEC1, | 0.859 | 0.848 | 0.869 | 0.921 | 0.868 | 0.974 | 0.939 | 0.863 | 1 |
| ISG15, HERC6, SLC1A2, FAM20A, MMP8 | | | | | | | | | |
| RSAD2, OAS1, IFIT1, IFI44L, SIGLEC1, | 0.859 | 0.849 | 0.868 | 0.921 | 0.869 | 0.974 | 0.950 | 0.892 | 1 |
| ISG15, SLC1A2, IL1R2, OLAH, FAM20A | | | | | | | | | |
| RSAD2, IFI27, OAS1, IFIT1, IFI44L, | 0.859 | 0.850 | 0.867 | 0.926 | 0.880 | 0.971 | 0.954 | 0.908 | 1 |
| SIGLEC1, ISG15, HERC6, SLC1A2, IL1R2 | | | | | | | | | |
| RSAD2, IFI27, IFI44L, SIGLEC1, ISG15, | 0.858 | 0.850 | 0.867 | 0.928 | 0.88 | 0.975 | 0.954 | 0.908 | 1 |
| HERC6, SLC1A2, IL1R2, RETN, MMP8 | | | | | | | | | |
| RSAD2, IFIT1, IFI44L, SIGLEC1, ISG15, | 0.858 | 0.849 | 0.868 | 0.926 | 0.878 | 0.974 | 0.941 | 0.869 | 1 |
| HERC6, SLC1A2, IL1R2, FAM20A, RETN | | | | | | | | | |
| IFI27, OAS1, IFIT1, IFI44L, ISG15, HERC6, | 0.858 | 0.849 | 0.868 | 0.924 | 0.874 | 0.974 | 0.936 | 0.870 | 1 |
| SLC1A2, IL1R2, RETN, MMP8 | | | | | | | | | |
| OAS1, IFIT1, IFI44L, SIGLEC1, ISG15, | 0.858 | 0.848 | 0.868 | 0.920 | 0.867 | 0.973 | 0.945 | 0.884 | 1 |
| SLC1A2, IL1R2, OLAH, FAM20A, RETN | | | | | | | | | |
| RSAD2, IFI27, OAS1, IFIT1, IFI44L, | 0.858 | 0.849 | 0.868 | 0.927 | 0.88 | 0.973 | 0.954 | 0.906 | 1 |
| SIGLEC1, HERC6, SLC1A2, IL1R2, MMP8 | | | | | | | | | |
| RSAD2, OASI, IFIT1, SIGLEC1, ISG15, | 0.858 | 0.848 | 0.869 | 0.922 | 0.869 | 0.975 | 0.951 | 0.891 | 1 |
| SLC1A2, OLAH, FAM20A, RETN, MMP8 | | | | | | | | | |
| RSAD2, IFI27, OAS1, IFI44L, ISG15, | 0.858 | 0.849 | 0.868 | 0.925 | 0.874 | 0.975 | 0.933 | 0.860 | 1 |
| HERC6, SLC1A2, IL1R2, RETN, MMP8 | | | | | | | | | |
| RSAD2, OAS1, IFIT1, IFI44L, SIGLEC1, | 0.858 | 0.848 | 0.868 | 0.924 | 0.875 | 0.974 | 0.934 | 0.864 | 1 |
| ISG15, HERC6, SLC1A2, FAM20A, RETN | | | | | | | | | |
| RSAD2, OAS1, IFIT1, IFI44L, SIGLEC1, | 0.858 | 0.848 | 0.868 | 0.926 | 0.879 | 0.974 | 0.945 | 0.878 | 1 |
| HERC6, SLC1A2, IL1R2, FAM20A, RETN | | | | | | | | | |
| RSAD2, OAS1, IFIT1, IFI44L, SIGLEC1, | 0.858 | 0.848 | 0.868 | 0.921 | 0.868 | 0.974 | 0.949 | 0.890 | 1 |
| SLC1A2, IL1R2, OLAH, FAM20A, RETN | | | | | | | | | |
| RSAD2, OAS1, IFIT1, SIGLEC1, ISG15, | 0.858 | 0.848 | 0.868 | 0.922 | 0.870 | 0.973 | 0.959 | 0.901 | 1 |
| SLC1A2, IL1R2, OLAH, FAM20A, RETN | | | | | | | | | |
| RSAD2, IFI27, OAS1, IFIT1, IFI44L, ISG15, | 0.858 | 0.847 | 0.869 | 0.926 | 0.877 | 0.976 | 0.922 | 0.834 | 1 |
| SLC1A2, IL1R2, FAM20A, RETN | | | | | | | | | |
| RSAD2, OAS1, IFI44L, SIGLEC1, ISG15, | 0.858 | 0.848 | 0.868 | 0.926 | 0.878 | 0.974 | 0.959 | 0.895 | 1 |
| SLC1A2, IL1R2, FAM20A, RETN, MMP8 | | | | | | | | | |
| RSAD2, IFI27, OAS1, IFIT1, IFI44L, ISG15, | 0.858 | 0.848 | 0.868 | 0.918 | 0.860 | 0.975 | 0.927 | 0.851 | 1 |
| HERC6, SLC1A2, RETN, MMP8 | | | | | | | | | |
| RSAD2, IFI27, OAS1, IFI44L, SIGLEC1, | 0.858 | 0.849 | 0.867 | 0.927 | 0.879 | 0.975 | 0.959 | 0.906 | 1 |
| ISG15, SLC1A2, IL1R2, RETN, MMP8 | | | | | | | | | |
| RSAD2, IFIT1, IFI44L, SIGLEC1, ISG15, | 0.858 | 0.848 | 0.867 | 0.922 | 0.869 | 0.974 | 0.950 | 0.890 | 1 |
| SLC1A2, IL1R2, OLAH, FAM20A, RETN | | | | | | | | | |
| RSAD2, OAS1, IFIT1, IFI44L, SIGLEC1, | 0.858 | 0.847 | 0.868 | 0.920 | 0.868 | 0.972 | 0.929 | 0.852 | 1 |
| ISG15, HERC6, SLC1A2, FAM20A, MMP8 | | | | | | | | | |
| RSAD2, OAS1, IFI44L, SIGLEC1, SLC1A2, | 0.858 | 0.847 | 0.868 | 0.923 | 0.874 | 0.973 | 0.959 | 0.899 | 1 |
| IL1R2, OLAH, FAM20A, RETN, MMP8 | | | | | | | | | |
| RSAD2, IFI27, OAS1, IFI44L, SIGLEC1, | 0.858 | 0.849 | 0.866 | 0.926 | 0.880 | 0.972 | 0.953 | 0.905 | 1 |
| ISG15, HERC6, SLC1A2, IL1R2, MMP8 | | | | | | | | | |
| RSAD2, OAS1, IFIT1, IFI44L, SIGLEC1, | 0.857 | 0.848 | 0.867 | 0.920 | 0.865 | 0.976 | 0.937 | 0.873 | 1 |
| ISG15, SLC1A2, OLAH, FAM20A, RETN | | | | | | | | | |
| RSAD2, OAS1, IFI44L, SIGLEC1, HERC6, | 0.857 | 0.847 | 0.868 | 0.921 | 0.867 | 0.974 | 0.941 | 0.872 | 1 |
| SLC1A2, OLAH, FAM20A, RETN, MMP8 | | | | | | | | | |
| RSAD2, IFI27, OAS1, IFIT1, SIGLEC1, | 0.857 | 0.848 | 0.866 | 0.926 | 0.877 | 0.974 | 0.961 | 0.908 | 1 |
| ISG15, SLC1A2, IL1R2, RETN, MMP8 | | | | | | | | | |
| RSAD2, IFI27, IFIT1, IFI44L, SIGLEC1, | 0.857 | 0.848 | 0.866 | 0.926 | 0.878 | 0.974 | 0.958 | 0.912 | 1 |
| ISG15, SLC1A2, IL1R2, RETN, MMP8 | | | | | | | | | |
| IFI27, OAS1, IFIT1, IFI44L, SIGLEC1, | 0.857 | 0.848 | 0.867 | 0.925 | 0.880 | 0.970 | 0.950 | 0.902 | 0.998 |
| ISG15, HERC6, SLC1A2, IL1R2, MMP8 | | | | | | | | | |
| RSAD2, IFI44L, SIGLEC1, ISG15, HERC6, | 0.857 | 0.847 | 0.867 | 0.920 | 0.866 | 0.974 | 0.941 | 0.870 | 1 |
| SLC1A2, OLAH, FAM20A, RETN, MMP8 | | | | | | | | | |
| RSAD2, OAS1, IFI44L, SIGLEC1, ISG15, | 0.856 | 0.846 | 0.866 | 0.916 | 0.856 | 0.976 | 0.951 | 0.893 | 1 |
| HERC6, OLAH, FAM20A, RETN, MMP8 | | | | | | | | | |
| RSAD2, OAS1, SIGLEC1, ISG15, HERC6, | 0.856 | 0.845 | 0.867 | 0.919 | 0.867 | 0.970 | 0.950 | 0.885 | 1 |
| SLC1A2, IL1R2, OLAH, FAM20A, RETN | | | | | | | | | |
| RSAD2, OAS1, IFI44L, SIGLEC1, ISG15, | 0.856 | 0.845 | 0.867 | 0.919 | 0.867 | 0.970 | 0.946 | 0.878 | 1 |
| HERC6, SLC1A2, IL1R2, OLAH, FAM20A | | | | | | | | | |
| RSAD2, OAS1, IFI44L, SIGLEC1, ISG15, | 0.856 | 0.845 | 0.866 | 0.913 | 0.854 | 0.972 | 0.959 | 0.905 | 1 |
| HERC6, IL1R2, OLAH, FAM20A, RETN | | | | | | | | | |
| RSAD2, IFI27, OAS1, IFIT1, IFI44L, | 0.856 | 0.847 | 0.864 | 0.927 | 0.881 | 0.972 | 0.954 | 0.907 | 1 |
| SIGLEC1, ISG15, SLC1A2, ILIR2, MMP8 | | | | | | | | | |
| RSAD2, IFI27, OAS1, IFIT1, SIGLEC1, | 0.856 | 0.846 | 0.865 | 0.926 | 0.881 | 0.972 | 0.952 | 0.903 | 1 |
| ISG15, HERC6, SLC1A2, IL1R2, MMP8 | | | | | | | | | |
| RSAD2, OAS1, IFI44L, SIGLEC1, ISG15, | 0.856 | 0.845 | 0.866 | 0.915 | 0.858 | 0.971 | 0.945 | 0.881 | 1 |
| SLC1A2, OLAH, FAM20A, RETN, MMP8 | | | | | | | | | |
| RSAD2, OAS1, IFIT1, IFI44L, SIGLEC1, | 0.855 | 0.846 | 0.865 | 0.921 | 0.870 | 0.972 | 0.954 | 0.894 | 1 |
| ISG15, SLC1A2, IL1R2, FAM20A, RETN | | | | | | | | | |
| OAS1, IFI44L, SIGLEC1, ISG15, HERC6, | 0.854 | 0.843 | 0.865 | 0.919 | 0.869 | 0.970 | 0.948 | 0.880 | 1 |
| SLC1A2, ILIR2, OLAH, FAM20A, RETN | | | | | | | | | |
| RSAD2, IFI27, IFIT1, IFI44L, SIGLEC1, | 0.854 | 0.845 | 0.863 | 0.923 | 0.871 | 0.975 | 0.945 | 0.890 | 1.000 |
| ISG15, HERC6, SLC1A2, RETN, MMP8 | | | | | | | | | |
| RSAD2, IFI27, IFIT1, IFI44L, SIGLEC1, | 0.854 | 0.845 | 0.863 | 0.925 | 0.879 | 0.971 | 0.950 | 0.902 | 0.998 |
| ISG15, HERC6, SLC1A2, IL1R2, MMP8 | | | | | | | | | |
| RSAD2, IFI27, IFIT1, IFI44L, ISG15, | 0.854 | 0.844 | 0.863 | 0.923 | 0.872 | 0.974 | 0.941 | 0.880 | 1 |
| HERC6, SLC1A2, IL1R2, RETN, MMP8 | | | | | | | | | |
| RSAD2, OAS1, IFI44L, SIGLEC1, ISG15, | 0.853 | 0.843 | 0.864 | 0.916 | 0.859 | 0.974 | 0.946 | 0.874 | 1 |
| HERC6, SLC1A2, FAM20A, RETN, MMP8 | | | | | | | | | |
| RSAD2, OAS1, IFI44L, SIGLEC1, HERC6, | 0.853 | 0.842 | 0.864 | 0.919 | 0.868 | 0.970 | 0.948 | 0.880 | 1 |
| SLC1A2, ILIR2, OLAH, FAM20A, RETN | | | | | | | | | |
| RSAD2, IFI27, OAS1, IFIT1, IFI44L, ISG15, | 0.853 | 0.844 | 0.862 | 0.920 | 0.868 | 0.973 | 0.938 | 0.872 | 1 |
| SLC1A2, IL1R2, RETN, MMP8 | | | | | | | | | |
| RSAD2, IFI44L, SIGLEC1, ISG15, HERC6, | 0.853 | 0.842 | 0.864 | 0.919 | 0.867 | 0.970 | 0.948 | 0.877 | 1 |
| SLC1A2, ILIR2, OLAH, FAM20A, RETN | | | | | | | | | |
| OAS1, IFIT1, IFI44L, ISG15, HERC6, | 0.852 | 0.842 | 0.863 | 0.922 | 0.870 | 0.974 | 0.921 | 0.834 | 1 |
| SLC1A2, OLAH, FAM20A, RETN, MMP8 | | | | | | | | | |
| RSAD2, IFI27, OAS1, IFIT1, IFI44L, ISG15, | 0.852 | 0.843 | 0.862 | 0.922 | 0.875 | 0.969 | 0.936 | 0.872 | 1 |
| HERC6, SLC1A2, IL1R2, MMP8 | | | | | | | | | |
| RSAD2, OAS1, IFI44L, SIGLEC1, ISG15, | 0.852 | 0.842 | 0.863 | 0.917 | 0.865 | 0.970 | 0.958 | 0.900 | 1 |
| SLC1A2, IL1R2, OLAH, FAM20A, RETN | | | | | | | | | |
| RSAD2, OAS1, IFIT1, IFI44L, HERC6, | 0.852 | 0.842 | 0.863 | 0.921 | 0.871 | 0.971 | 0.927 | 0.845 | 1 |
| SLC1A2, IL1R2, OLAH, FAM20A, MMP8 | | | | | | | | | |
| RSAD2, OAS1, IFI44L, SIGLEC1, ISG15, | 0.852 | 0.841 | 0.863 | 0.919 | 0.869 | 0.970 | 0.951 | 0.883 | 1 |
| HERC6, SLC1A2, IL1R2, FAM20A, RETN | | | | | | | | | |
| RSAD2, OAS1, IFIT1, IFI44L, HERC6, | 0.852 | 0.842 | 0.862 | 0.920 | 0.867 | 0.972 | 0.922 | 0.838 | 1 |
| SLC1A2, OLAH, FAM20A, RETN, MMP8 | | | | | | | | | |
| RSAD2, OAS1, IFI44L, SIGLEC1, ISG15, | 0.852 | 0.841 | 0.863 | 0.917 | 0.863 | 0.972 | 0.941 | 0.872 | 1 |
| HERC6, SLC1A2, OLAH, FAM20A, RETN | | | | | | | | | |
| RSAD2, IFI27, OAS1, IFIT1, IFI44L, ISG15, | 0.852 | 0.840 | 0.863 | 0.918 | 0.863 | 0.972 | 0.911 | 0.822 | 1 |
| HERC6, SLC1A2, FAM20A, MMP8 | | | | | | | | | |
| RSAD2, OAS1, IFIT1, IFI44L, ISG15, | 0.852 | 0.841 | 0.863 | 0.919 | 0.867 | 0.971 | 0.918 | 0.836 | 1 |
| HERC6, SLC1A2, OLAH, FAM20A, MMP8 | | | | | | | | | |
| RSAD2, OAS1, IFI44L, ISG15, HERC6, | 0.851 | 0.840 | 0.862 | 0.916 | 0.858 | 0.974 | 0.939 | 0.865 | 1 |
| IL1R2, OLAH, FAM20A, RETN, MMP8 | | | | | | | | | |
| RSAD2, OAS1, IFI44L, ISG15, HERC6, | 0.851 | 0.840 | 0.862 | 0.919 | 0.868 | 0.970 | 0.927 | 0.845 | 1 |
| SLC1A2, IL1R2, OLAH, FAM20A, MMP8 | | | | | | | | | |
| RSAD2, IFI27, OAS1, IFIT1, IFI44L, | 0.851 | 0.841 | 0.861 | 0.915 | 0.858 | 0.973 | 0.947 | 0.877 | 1 |
| SIGLEC1, ISG15, SLC1A2, RETN, MMP8 | | | | | | | | | |
| RSAD2, OAS1, IFIT1, IFI44L, ISG15, | 0.851 | 0.840 | 0.862 | 0.912 | 0.854 | 0.970 | 0.932 | 0.853 | 1 |
| HERC6, IL1R2, OLAH, FAM20A, MMP8 | | | | | | | | | |
| OAS1, IFIT1, IFI44L, ISG15, HERC6, | 0.851 | 0.840 | 0.861 | 0.918 | 0.866 | 0.969 | 0.927 | 0.844 | 1 |
| SLC1A2, IL1R2, OLAH, FAM20A, MMP8 | | | | | | | | | |
| IFIT1, IFI44L, ISG15, HERC6, SLC1A2, | 0.850 | 0.840 | 0.861 | 0.920 | 0.869 | 0.972 | 0.927 | 0.842 | 1 |
| IL1R2, OLAH, FAM20A, RETN, MMP8 | | | | | | | | | |
| RSAD2, OAS1, IFI44L, HERC6, SLC1A2, | 0.850 | 0.840 | 0.861 | 0.919 | 0.867 | 0.971 | 0.929 | 0.847 | 1 |
| IL1R2, OLAH, FAM20A, RETN, MMP8 | | | | | | | | | |
| RSAD2, OAS1, IFIT1, IFI44L, HERC6, | 0.850 | 0.840 | 0.861 | 0.913 | 0.855 | 0.972 | 0.937 | 0.861 | 1 |
| IL1R2, OLAH, FAM20A, RETN, MMP8 | | | | | | | | | |
| RSAD2, OAS1, IFIT1, IFI44L, SIGLEC1, | 0.850 | 0.840 | 0.861 | 0.918 | 0.862 | 0.974 | 0.938 | 0.867 | 1 |
| ISG15, SLC1A2, FAM20A, RETN, MMP8 | | | | | | | | | |
| RSAD2, IFIT1, IFI44L, HERC6, SLC1A2, | 0.850 | 0.840 | 0.860 | 0.921 | 0.869 | 0.972 | 0.925 | 0.841 | 1 |
| IL1R2, OLAH, FAM20A, RETN, MMP8 | | | | | | | | | |
| RSAD2, OAS1, IFIT1, ISG15, HERC6, | 0.850 | 0.840 | 0.860 | 0.911 | 0.850 | 0.971 | 0.942 | 0.869 | 1 |
| IL1R2, OLAH, FAM20A, RETN, MMP8 | | | | | | | | | |

Performance obtained for the identification of the viral or bacterial nature of the infection on the basis of the target 11-gene combinations:

TABLE 15

| | AUC | | | AUC | CI 95% | | AUC | CI 95% | |
|---|---|---|---|---|---|---|---|---|---|
| Model | model | CI 95% | | Train | Train | | Test | Test | |
| RSAD2, IFI27, OAS1, IFIT1, IFI44L, SIGLEC1, | 0.880 | 0.870 | 0.889 | 0.938 | 0.889 | 0.987 | 0.951 | 0.888 | 1 |
| HERC6, IL1R2, OLAH, FAM20A, MMP8 | | | | | | | | | |
| RSAD2, IFI27, OAS1, IFIT1, SIGLEC1, ISG15, | 0.879 | 0.869 | 0.889 | 0.938 | 0.889 | 0.987 | 0.952 | 0.891 | 1 |
| HERC6, IL1R2, OLAH, FAM20A, MMP8 | | | | | | | | | |
| IFI27, OAS1, IFIT1, IFI44L, SIGLEC1, HERC6, | 0.879 | 0.869 | 0.889 | 0.939 | 0.888 | 0.990 | 0.951 | 0.889 | 1 |
| IL1R2, OLAH, FAM20A, RETN, MMP8 | | | | | | | | | |
| IFI27, OAS1, IFIT1, SIGLEC1, ISG15, HERC6, | 0.879 | 0.868 | 0.889 | 0.939 | 0.888 | 0.990 | 0.953 | 0.893 | 1 |
| IL1R2, OLAH, FAM20A, RETN, MMP8 | | | | | | | | | |
| IFI27, OAS1, IFIT1, IFI44L, SIGLEC1, ISG15, | 0.878 | 0.868 | 0.889 | 0.938 | 0.889 | 0.986 | 0.947 | 0.882 | 1 |
| HERC6, IL1R2, OLAH, FAM20A, MMP8 | | | | | | | | | |
| RSAD2, IFI27, OAS1, IFIT1, SIGLEC1, HERC6, | 0.878 | 0.868 | 0.889 | 0.938 | 0.886 | 0.989 | 0.949 | 0.885 | 1 |
| IL1R2, OLAH, FAM20A, RETN, MMP8 | | | | | | | | | |
| RSAD2, IFI27, OAS1, IFI44L, SIGLEC1, ISG15, | 0.877 | 0.866 | 0.888 | 0.938 | 0.889 | 0.986 | 0.947 | 0.883 | 1 |
| HERC6, IL1R2, OLAH, FAM20A, MMP8 | | | | | | | | | |
| RSAD2, IFI27, OAS1, IFIT1, IFI44L, SIGLEC1, | 0.877 | 0.866 | 0.887 | 0.936 | 0.883 | 0.989 | 0.946 | 0.882 | 1 |
| HERC6, OLAH, FAM20A, RETN, MMP8 | | | | | | | | | |
| RSAD2, IFI27, IFIT1, IFI44L, SIGLEC1, ISG15, | 0.877 | 0.866 | 0.887 | 0.938 | 0.890 | 0.986 | 0.947 | 0.883 | 1 |
| HERC6, IL1R2, OLAH, FAM20A, MMP8 | | | | | | | | | |
| RSAD2, IFI27, OAS1, IFIT1, SIGLEC1, ISG15, | 0.876 | 0.865 | 0.887 | 0.937 | 0.884 | 0.989 | 0.949 | 0.887 | 1 |
| HERC6, OLAH, FAM20A, RETN, MMP8 | | | | | | | | | |
| RSAD2, IFI27, OAS1, SIGLEC1, ISG15, HERC6, | 0.876 | 0.865 | 0.887 | 0.937 | 0.885 | 0.988 | 0.949 | 0.885 | 1 |
| IL1R2, OLAH, FAM20A, RETN, MMP8 | | | | | | | | | |
| IFI27, IFIT1, IFI44L, SIGLEC1, ISG15, HERC6, | 0.876 | 0.866 | 0.887 | 0.938 | 0.886 | 0.989 | 0.949 | 0.884 | 1 |
| IL1R2, OLAH, FAM20A, RETN, MMP8 | | | | | | | | | |
| IFI27, OAS1, IFI44L, SIGLEC1, ISG15, HERC6, | 0.876 | 0.866 | 0.887 | 0.937 | 0.886 | 0.989 | 0.948 | 0.883 | 1 |
| IL1R2, OLAH, FAM20A, RETN, MMP8 | | | | | | | | | |
| RSAD2, IFI27, OAS1, IFIT1, IFI44L, SIGLEC1, | 0.876 | 0.866 | 0.887 | 0.936 | 0.887 | 0.986 | 0.951 | 0.890 | 1 |
| ISG15, IL1R2, OLAH, FAM20A, MMP8 | | | | | | | | | |
| IFI27, OAS1, IFIT1, IFI44L, SIGLEC1, ISG15, | 0.875 | 0.864 | 0.887 | 0.936 | 0.884 | 0.989 | 0.946 | 0.881 | 1 |
| HERC6, OLAH, FAM20A, RETN, MMP8 | | | | | | | | | |
| RSAD2, IFI27, IFIT1, SIGLEC1, ISG15, HERC6, | 0.875 | 0.864 | 0.886 | 0.937 | 0.886 | 0.988 | 0.950 | 0.888 | 1 |
| IL1R2, OLAH, FAM20A, RETN, MMP8 | | | | | | | | | |
| RSAD2, IFI27, OAS1, IFI44L, SIGLEC1, HERC6, | 0.875 | 0.864 | 0.886 | 0.937 | 0.885 | 0.989 | 0.949 | 0.884 | 1 |
| IL1R2, OLAH, FAM20A, RETN, MMP8 | | | | | | | | | |
| IFI27, OAS1, IFIT1, IFI44L, SIGLEC1, ISG15, | 0.875 | 0.864 | 0.886 | 0.937 | 0.888 | 0.988 | 0.950 | 0.881 | 1 |
| HERC6, IL1R2, FAM20A, RETN, MMP8 | | | | | | | | | |
| RSAD2, IFI27, OAS1, IFIT1, SIGLEC1, ISG15, | 0.875 | 0.865 | 0.885 | 0.937 | 0.887 | 0.988 | 0.952 | 0.885 | 1 |
| HERC6, IL1R2, FAM20A, RETN, MMP8 | | | | | | | | | |
| RSAD2, IFI27, IFIT1, IFI44L, SIGLEC1, HERC6, | 0.875 | 0.864 | 0.886 | 0.937 | 0.886 | 0.989 | 0.951 | 0.888 | 1 |
| IL1R2, OLAH, FAM20A, RETN, MMP8 | | | | | | | | | |
| RSAD2, IFI27, OAS1, IFIT1, SIGLEC1, ISG15, | 0.874 | 0.865 | 0.884 | 0.933 | 0.880 | 0.986 | 0.939 | 0.875 | 1 |
| HERC6, IL1R2, OLAH, FAM20A, RETN | | | | | | | | | |
| RSAD2, IFI27, OAS1, IFIT1, IFI44L, SIGLEC1, | 0.874 | 0.865 | 0.884 | 0.933 | 0.881 | 0.984 | 0.941 | 0.876 | 1 |
| ISG15, HERC6, IL1R2, OLAH, FAM20A | | | | | | | | | |
| RSAD2, IFI27, OAS1, IFIT1, IFI44L, SIGLEC1, | 0.874 | 0.863 | 0.885 | 0.938 | 0.889 | 0.988 | 0.951 | 0.882 | 1 |
| HERC6, IL1R2, FAM20A, RETN, MMP8 | | | | | | | | | |
| RSAD2, IFI27, IFIT1, SIGLEC1, ISG15, HERC6, | 0.874 | 0.864 | 0.884 | 0.944 | 0.901 | 0.986 | 0.950 | 0.886 | 1 |
| SLC1A2, IL1R2, OLAH, FAM20A, MMP8 | | | | | | | | | |
| RSAD2, IFI27, OAS1, IFIT1, IFI44L, SIGLEC1, | 0.874 | 0.864 | 0.884 | 0.930 | 0.877 | 0.984 | 0.940 | 0.876 | 1 |
| HERC6, IL1R2, OLAH, FAM20A, RETN | | | | | | | | | |
| RSAD2, IFI27, OAS1, IFIT1, SIGLEC1, HERC6, | 0.874 | 0.863 | 0.884 | 0.941 | 0.897 | 0.985 | 0.947 | 0.882 | 1 |
| SLC1A2, IL1R2, OLAH, FAM20A, MMP8 | | | | | | | | | |
| RSAD2, IFI27, OAS1, IFIT1, SIGLEC1, ISG15, | 0.874 | 0.863 | 0.885 | 0.935 | 0.883 | 0.988 | 0.953 | 0.892 | 1 |
| IL1R2, OLAH, FAM20A, RETN, MMP8 | | | | | | | | | |
| RSAD2, IFI27, OAS1, IFIT1, IFI44L, SIGLEC1, | 0.873 | 0.862 | 0.885 | 0.934 | 0.881 | 0.986 | 0.945 | 0.883 | 1 |
| ISG15, HERC6, OLAH, FAM20A, MMP8 | | | | | | | | | |
| IFI27, IFIT1, IFI44L, SIGLEC1, ISG15, HERC6, | 0.873 | 0.863 | 0.883 | 0.943 | 0.900 | 0.986 | 0.949 | 0.883 | 1 |
| SLC1A2, IL1R2, OLAH, FAM20A, MMP8 | | | | | | | | | |
| IFI27, OAS1, IFIT1, IFI44L, SIGLEC1, ISG15, | 0.873 | 0.863 | 0.883 | 0.931 | 0.878 | 0.985 | 0.938 | 0.872 | 1 |
| HERC6, IL1R2, OLAH, FAM20A, RETN | | | | | | | | | |
| IFI27, OAS1, IFIT1, SIGLEC1, ISG15, HERC6, | 0.873 | 0.862 | 0.885 | 0.942 | 0.899 | 0.986 | 0.947 | 0.879 | 1 |
| SLC1A2, IL1R2, OLAH, FAM20A, MMP8 | | | | | | | | | |
| RSAD2, IFI27, OAS1, IFIT1, SIGLEC1, ISG15, | 0.873 | 0.863 | 0.883 | 0.943 | 0.900 | 0.986 | 0.947 | 0.880 | 1 |
| SLC1A2, IL1R2, OLAH, FAM20A, MMP8 | | | | | | | | | |
| RSAD2, IFI27, OAS1, IFIT1, IFI44L, SIGLEC1, | 0.873 | 0.863 | 0.883 | 0.931 | 0.878 | 0.985 | 0.940 | 0.876 | 1 |
| ISG15, HERC6, OLAH, FAM20A, RETN | | | | | | | | | |
| IFI27, OAS1, IFIT1, IFI44L, SIGLEC1, ISG15, | 0.873 | 0.861 | 0.885 | 0.936 | 0.884 | 0.988 | 0.950 | 0.887 | 1 |
| IL1R2, OLAH, FAM20A, RETN, MMP8 | | | | | | | | | |
| IFI27, OAS1, IFIT1, IFI44L, SIGLEC1, HERC6, | 0.873 | 0.862 | 0.884 | 0.941 | 0.896 | 0.985 | 0.946 | 0.880 | 1 |
| SLC1A2, IL1R2, OLAH, FAM20A, MMP8 | | | | | | | | | |
| IFI27, OAS1, IFIT1, IFI44L, SIGLEC1, HERC6, | 0.873 | 0.863 | 0.883 | 0.940 | 0.896 | 0.984 | 0.946 | 0.874 | 1 |
| SLC1A2, IL1R2, FAM20A, RETN, MMP8 | | | | | | | | | |
| IFI27, OAS1, SIGLEC1, ISG15, HERC6, SLC1A2, | 0.873 | 0.862 | 0.883 | 0.941 | 0.897 | 0.986 | 0.946 | 0.877 | 1 |
| IL1R2, OLAH, FAM20A, RETN, MMP8 | | | | | | | | | |
| RSAD2, IFI27, OAS1, IFIT1, SIGLEC1, ISG15, | 0.873 | 0.862 | 0.884 | 0.941 | 0.896 | 0.986 | 0.941 | 0.876 | 1 |
| HERC6, SLC1A2, OLAH, FAM20A, MMP8 | | | | | | | | | |
| RSAD2, IFI27, OAS1, IFIT1, IFI44L, SIGLEC1, | 0.873 | 0.861 | 0.884 | 0.939 | 0.892 | 0.986 | 0.942 | 0.879 | 1 |
| HERC6, SLC1A2, OLAH, FAM20A, MMP8 | | | | | | | | | |
| IFI27, OAS1, IFIT1, SIGLEC1, ISG15, HERC6, | 0.873 | 0.862 | 0.884 | 0.942 | 0.898 | 0.987 | 0.943 | 0.878 | 1 |
| SLC1A2, OLAH, FAM20A, RETN, MMP8 | | | | | | | | | |
| IFI27, OAS1, IFIT1, SIGLEC1, HERC6, SLC1A2, | 0.872 | 0.861 | 0.884 | 0.942 | 0.898 | 0.986 | 0.942 | 0.874 | 1 |
| IL1R2, OLAH, FAM20A, RETN, MMP8 | | | | | | | | | |
| IFI27, OAS1, IFI44L, SIGLEC1, ISG15, HERC6, | 0.872 | 0.861 | 0.883 | 0.94 | 0.897 | 0.984 | 0.947 | 0.879 | 1 |
| SLC1A2, IL1R2, OLAH, FAM20A, MMP8 | | | | | | | | | |
| RSAD2, IFI27, OAS1, IFIT1, IFI44L, SIGLEC1, | 0.872 | 0.862 | 0.882 | 0.933 | 0.884 | 0.982 | 0.950 | 0.882 | 1 |
| ISG15, HERC6, IL1R2, FAM20A, MMP8 | | | | | | | | | |
| RSAD2, IFI27, IFIT1, IFI44L, SIGLEC1, HERC6, | 0.872 | 0.862 | 0.883 | 0.94 | 0.895 | 0.985 | 0.949 | 0.885 | 1 |
| SLC1A2, IL1R2, OLAH, FAM20A, MMP8 | | | | | | | | | |
| RSAD2, IFI27, OAS1, SIGLEC1, ISG15, HERC6, | 0.872 | 0.861 | 0.884 | 0.94 | 0.896 | 0.985 | 0.947 | 0.879 | 1 |
| SLC1A2, IL1R2, OLAH, FAM20A, MMP8 | | | | | | | | | |
| IFI27, OAS1, IFIT1, IFI44L, SIGLEC1, ISG15, | 0.872 | 0.861 | 0.883 | 0.942 | 0.898 | 0.985 | 0.948 | 0.882 | 1 |
| SLC1A2, IL1R2, OLAH, FAM20A, MMP8 | | | | | | | | | |
| RSAD2, IFI27, IFIT1, IFI44L, SIGLEC1, ISG15, | 0.872 | 0.862 | 0.882 | 0.933 | 0.880 | 0.986 | 0.943 | 0.879 | 1 |
| HERC6, IL1R2, OLAH, FAM20A, RETN | | | | | | | | | |
| RSAD2, IFI27, OAS1, IFIT1, IFI44L, SIGLEC1, | 0.872 | 0.861 | 0.882 | 0.939 | 0.891 | 0.987 | 0.945 | 0.880 | 1 |
| ISG15, SLC1A2, OLAH, FAM20A, MMP8 | | | | | | | | | |
| RSAD2, IFI27, OAS1, IFIT1, IFI44L, HERC6, | 0.871 | 0.860 | 0.882 | 0.933 | 0.880 | 0.986 | 0.930 | 0.857 | 1 |
| IL1R2, OLAH, FAM20A, RETN, MMP8 | | | | | | | | | |
| IFI27, OAS1, IFIT1, IFI44L, SIGLEC1, ISG15, | 0.871 | 0.860 | 0.883 | 0.942 | 0.897 | 0.987 | 0.938 | 0.871 | 1 |
| HERC6, SLC1A2, OLAH, FAM20A, MMP8 | | | | | | | | | |
| IFI27, OAS1, IFIT1, IFI44L, SIGLEC1, HERC6, | 0.871 | 0.859 | 0.883 | 0.940 | 0.894 | 0.986 | 0.943 | 0.878 | 1 |
| SLC1A2, OLAH, FAM20A, RETN, MMP8 | | | | | | | | | |
| RSAD2, IFI27, OAS1, IFI44L, SIGLEC1, ISG15, | 0.871 | 0.860 | 0.882 | 0.936 | 0.884 | 0.989 | 0.950 | 0.887 | 1 |
| IL1R2, OLAH, FAM20A, RETN, MMP8 | | | | | | | | | |
| RSAD2, IFI27, OAS1, IFIT1, IFI44L, SIGLEC1, | 0.871 | 0.861 | 0.882 | 0.939 | 0.893 | 0.985 | 0.946 | 0.879 | 1 |
| SLC1A2, IL1R2, OLAH, FAM20A, MMP8 | | | | | | | | | |
| IFI27, OAS1, IFIT1, SIGLEC1, ISG15, SLC1A2, | 0.871 | 0.860 | 0.882 | 0.942 | 0.897 | 0.986 | 0.947 | 0.880 | 1 |
| IL1R2, OLAH, FAM20A, RETN, MMP8 | | | | | | | | | |
| RSAD2, IFI27, IFIT1, IFI44L, SIGLEC1, ISG15, | 0.871 | 0.860 | 0.882 | 0.938 | 0.888 | 0.988 | 0.950 | 0.880 | 1 |
| HERC6, IL1R2, FAM20A, RETN, MMP8 | | | | | | | | | |
| RSAD2, IFI27, IFIT1, IFI44L, SIGLEC1, ISG15, | 0.871 | 0.860 | 0.881 | 0.943 | 0.900 | 0.986 | 0.949 | 0.883 | 1 |
| SLC1A2, IL1R2, OLAH, FAM20A, MMP8 | | | | | | | | | |
| RSAD2, IFI27, OAS1, IFIT1, SIGLEC1, HERC6, | 0.871 | 0.859 | 0.882 | 0.940 | 0.894 | 0.986 | 0.942 | 0.875 | 1 |
| SLC1A2, OLAH, FAM20A, RETN, MMP8 | | | | | | | | | |
| RSAD2, IFI27, IFIT1, IFI44L, SIGLEC1, ISG15, | 0.871 | 0.859 | 0.882 | 0.936 | 0.884 | 0.988 | 0.951 | 0.889 | 1 |
| IL1R2, OLAH, FAM20A, RETN, MMP8 | | | | | | | | | |
| RSAD2, IFI27, OAS1, SIGLEC1, HERC6, | 0.871 | 0.859 | 0.882 | 0.940 | 0.894 | 0.986 | 0.945 | 0.878 | 1 |
| SLC1A2, IL1R2, OLAH, FAM20A, RETN, MMP8 | | | | | | | | | |
| RSAD2, IFI27, OAS1, IFIT1, IFI44L, SIGLEC1, | 0.870 | 0.858 | 0.882 | 0.935 | 0.882 | 0.988 | 0.952 | 0.893 | 1 |
| IL1R2, OLAH, FAM20A, RETN, MMP8 | | | | | | | | | |
| RSAD2, IFI27, OAS1, IFIT1, SIGLEC1, ISG15, | 0.870 | 0.860 | 0.881 | 0.939 | 0.893 | 0.984 | 0.934 | 0.864 | 1 |
| HERC6, SLC1A2, IL1R2, OLAH, FAM20A | | | | | | | | | |
| RSAD2, IFI27, IFI44L, SIGLEC1, ISG15, HERC6, | 0.870 | 0.858 | 0.882 | 0.938 | 0.888 | 0.989 | 0.949 | 0.885 | 1 |
| IL1R2, OLAH, FAM20A, RETN, MMP8 | | | | | | | | | |
| RSAD2, IFI27, IFI44L, SIGLEC1, ISG15, HERC6, | 0.870 | 0.859 | 0.882 | 0.942 | 0.898 | 0.985 | 0.950 | 0.885 | 1 |
| SLC1A2, IL1R2, OLAH, FAM20A, MMP8 | | | | | | | | | |
| RSAD2, IFI27, OAS1, IFIT1, IFI44L, ISG15, | 0.870 | 0.859 | 0.882 | 0.932 | 0.881 | 0.983 | 0.932 | 0.861 | 1 |
| HERC6, IL1R2, OLAH, FAM20A, MMP8 | | | | | | | | | |
| RSAD2, IFI27, IFIT1, IFI44L, SIGLEC1, ISG15, | 0.870 | 0.859 | 0.882 | 0.935 | 0.882 | 0.988 | 0.950 | 0.888 | 1 |
| HERC6, OLAH, FAM20A, RETN, MMP8 | | | | | | | | | |
| IFI27, OAS1, IFIT1, SIGLEC1, ISG15, HERC6, | 0.870 | 0.859 | 0.881 | 0.94 | 0.897 | 0.983 | 0.949 | 0.877 | 1 |
| SLC1A2, IL1R2, FAM20A, RETN, MMP8 | | | | | | | | | |
| IFI27, OAS1, IFIT1, IFI44L, ISG15, HERC6, | 0.870 | 0.859 | 0.881 | 0.932 | 0.878 | 0.985 | 0.928 | 0.853 | 1 |
| IL1R2, OLAH, FAM20A, RETN, MMP8 | | | | | | | | | |
| RSAD2, IFI27, OAS1, IFI44L, SIGLEC1, ISG15, | 0.870 | 0.859 | 0.881 | 0.933 | 0.880 | 0.985 | 0.942 | 0.876 | 1 |
| HERC6, IL1R2, OLAH, FAM20A, RETN | | | | | | | | | |
| IFI27, OAS1, IFIT1, IFI44L, SIGLEC1, ISG15, | 0.870 | 0.860 | 0.880 | 0.940 | 0.895 | 0.984 | 0.932 | 0.861 | 1 |
| HERC6, SLC1A2, IL1R2, OLAH, FAM20A | | | | | | | | | |
| IFI27, IFIT1, IFI44L, SIGLEC1, ISG15, SLC1A2, | 0.870 | 0.859 | 0.881 | 0.944 | 0.901 | 0.987 | 0.948 | 0.880 | 1 |
| IL1R2, OLAH, FAM20A, RETN, MMP8 | | | | | | | | | |
| RSAD2, IFI27, OAS1, IFIT1, ISG15, HERC6, | 0.870 | 0.859 | 0.881 | 0.933 | 0.880 | 0.986 | 0.930 | 0.858 | 1 |
| IL1R2, OLAH, FAM20A, RETN, MMP8 | | | | | | | | | |
| IFI27, OAS1, IFI44L, SIGLEC1, HERC6, SLC1A2, | 0.870 | 0.859 | 0.881 | 0.939 | 0.893 | 0.985 | 0.948 | 0.882 | 1 |
| IL1R2, OLAH, FAM20A, RETN, MMP8 | | | | | | | | | |
| RSAD2, IFI27, IFIT1, SIGLEC1, ISG15, SLC1A2, | 0.870 | 0.859 | 0.881 | 0.943 | 0.900 | 0.986 | 0.947 | 0.879 | 1 |
| IL1R2, OLAH, FAM20A, RETN, MMP8 | | | | | | | | | |
| RSAD2, IFI27, OAS1, IFIT1, IFI44L, SIGLEC1, | 0.870 | 0.859 | 0.881 | 0.933 | 0.879 | 0.986 | 0.942 | 0.873 | 1 |
| ISG15, HERC6, FAM20A, RETN, MMP8 | | | | | | | | | |
| RSAD2, IFI27, OAS1, IFIT1, IFI44L, SIGLEC1, | 0.870 | 0.859 | 0.880 | 0.937 | 0.890 | 0.984 | 0.934 | 0.864 | 1 |
| ISG15, HERC6, SLC1A2, OLAH, FAM20A | | | | | | | | | |
| IFI27, IFI44L, SIGLEC1, ISG15, HERC6, SLC1A2, | 0.870 | 0.859 | 0.880 | 0.941 | 0.897 | 0.985 | 0.947 | 0.877 | 1 |
| IL1R2, OLAH, FAM20A, RETN, MMP8 | | | | | | | | | |
| RSAD2, IFI27, OAS1, IFI44L, SIGLEC1, ISG15, | 0.870 | 0.858 | 0.881 | 0.935 | 0.882 | 0.989 | 0.947 | 0.883 | 1 |
| HERC6, OLAH, FAM20A, RETN, MMP8 | | | | | | | | | |
| RSAD2, IFI27, IFIT1, SIGLEC1, ISG15, HERC6, | 0.870 | 0.859 | 0.880 | 0.943 | 0.899 | 0.987 | 0.948 | 0.884 | 1 |
| SLC1A2, OLAH, FAM20A, RETN, MMP8 | | | | | | | | | |
| RSAD2, IFI27, IFIT1, IFI44L, SIGLEC1, ISG15, | 0.869 | 0.858 | 0.881 | 0.941 | 0.895 | 0.987 | 0.943 | 0.880 | 1 |
| HERC6, SLC1A2, OLAH, FAM20A, MMP8 | | | | | | | | | |
| RSAD2, IFI27, SIGLEC1, ISG15, HERC6, | 0.869 | 0.858 | 0.881 | 0.944 | 0.902 | 0.987 | 0.948 | 0.881 | 1 |
| SLC1A2, IL1R2, OLAH, FAM20A, RETN, MMP8 | | | | | | | | | |
| RSAD2, IFI27, OAS1, IFI44L, SIGLEC1, ISG15, | 0.869 | 0.858 | 0.881 | 0.940 | 0.897 | 0.984 | 0.947 | 0.879 | 1 |
| SLC1A2, IL1R2, OLAH, FAM20A, MMP8 | | | | | | | | | |
| IFI27, OAS1, IFI44L, SIGLEC1, ISG15, HERC6, | 0.869 | 0.858 | 0.881 | 0.940 | 0.894 | 0.986 | 0.943 | 0.877 | 1 |
| SLC1A2, OLAH, FAM20A, RETN, MMP8 | | | | | | | | | |
| RSAD2, IFI27, OAS1, IFIT1, IFI44L, SIGLEC1, | 0.869 | 0.859 | 0.879 | 0.935 | 0.886 | 0.984 | 0.946 | 0.879 | 1 |
| ISG15, HERC6, IL1R2, FAM20A, RETN | | | | | | | | | |
| IFI27, OAS1, IFIT1, IFI44L, SIGLEC1, HERC6, | 0.869 | 0.859 | 0.880 | 0.937 | 0.891 | 0.984 | 0.936 | 0.869 | 1 |
| SLC1A2, IL1R2, OLAH, FAM20A, RETN | | | | | | | | | |
| RSAD2, IFI27, OAS1, IFI44L, SIGLEC1, ISG15, | 0.869 | 0.858 | 0.881 | 0.938 | 0.887 | 0.988 | 0.948 | 0.877 | 1 |
| HERC6, IL1R2, FAM20A, RETN, MMP8 | | | | | | | | | |
| IFI27, IFIT1, SIGLEC1, ISG15, HERC6, SLC1A2, | 0.869 | 0.858 | 0.880 | 0.943 | 0.899 | 0.986 | 0.949 | 0.881 | 1 |
| IL1R2, OLAH, FAM20A, RETN, MMP8 | | | | | | | | | |
| IFI27, OAS1, IFI44L, SIGLEC1, ISG15, SLC1A2, | 0.869 | 0.858 | 0.880 | 0.941 | 0.898 | 0.985 | 0.945 | 0.875 | 1 |
| IL1R2, OLAH, FAM20A, RETN, MMP8 | | | | | | | | | |
| RSAD2, IFI27, OAS1, IFIT1, IFI44L, ISG15, | 0.869 | 0.857 | 0.881 | 0.931 | 0.877 | 0.986 | 0.928 | 0.855 | 1 |
| HERC6, OLAH, FAM20A, RETN, MMP8 | | | | | | | | | |
| RSAD2, IFI27, IFIT1, SIGLEC1, HERC6, SLC1A2, | 0.869 | 0.858 | 0.880 | 0.940 | 0.895 | 0.986 | 0.950 | 0.886 | 1 |
| IL1R2, OLAH, FAM20A, RETN, MMP8 | | | | | | | | | |
| RSAD2, IFI27, OAS1, IFI44L, ISG15, HERC6, | 0.869 | 0.857 | 0.881 | 0.933 | 0.881 | 0.985 | 0.933 | 0.861 | 1 |
| IL1R2, OLAH, FAM20A, RETN, MMP8 | | | | | | | | | |
| IFI27, OAS1, IFIT1, IFI44L, SIGLEC1, ISG15, | 0.869 | 0.858 | 0.879 | 0.939 | 0.895 | 0.984 | 0.932 | 0.861 | 1 |
| HERC6, SLC1A2, OLAH, FAM20A, RETN | | | | | | | | | |
| RSAD2, IFI27, OAS1, IFI44L, SIGLEC1, HERC6, | 0.869 | 0.857 | 0.881 | 0.937 | 0.891 | 0.984 | 0.945 | 0.878 | 1 |
| SLC1A2, IL1R2, OLAH, FAM20A, MMP8 | | | | | | | | | |
| RSAD2, IFI27, OAS1, IFI44L, SIGLEC1, ISG15, | 0.869 | 0.857 | 0.880 | 0.937 | 0.889 | 0.985 | 0.942 | 0.877 | 1 |
| HERC6, SLC1A2, OLAH, FAM20A, MMP8 | | | | | | | | | |
| IFI27, OAS1, IFIT1, SIGLEC1, ISG15, HERC6, | 0.869 | 0.858 | 0.880 | 0.940 | 0.895 | 0.984 | 0.932 | 0.861 | 1 |
| SLC1A2, IL1R2, OLAH, FAM20A, RETN | | | | | | | | | |
| IFI27, IFIT1, IFI44L, SIGLEC1, ISG15, HERC6, | 0.869 | 0.857 | 0.880 | 0.941 | 0.895 | 0.986 | 0.950 | 0.886 | 1 |
| SLC1A2, OLAH, FAM20A, RETN, MMP8 | | | | | | | | | |
| RSAD2, IFI27, OAS1, IFIT1, IFI44L, SIGLEC1, | 0.869 | 0.858 | 0.880 | 0.936 | 0.892 | 0.980 | 0.950 | 0.882 | 1 |
| HERC6, SLC1A2, IL1R2, FAM20A, MMP8 | | | | | | | | | |
| RSAD2, IFI27, OAS1, IFIT1, IFI44L, SIGLEC1, | 0.869 | 0.858 | 0.879 | 0.929 | 0.874 | 0.984 | 0.947 | 0.886 | 1 |
| ISG15, IL1R2, OLAH, FAM20A, RETN | | | | | | | | | |
| RSAD2, IFI27, OAS1, IFIT1, IFI44L, SIGLEC1, | 0.869 | 0.858 | 0.879 | 0.936 | 0.890 | 0.983 | 0.938 | 0.871 | 1 |
| HERC6, SLC1A2, IL1R2, OLAH, FAM20A | | | | | | | | | |
| RSAD2, IFI27, IFIT1, IFI44L, SIGLEC1, HERC6, | 0.868 | 0.858 | 0.879 | 0.940 | 0.894 | 0.986 | 0.950 | 0.881 | 1 |
| SLC1A2, IL1R2, FAM20A, RETN, MMP8 | | | | | | | | | |
| RSAD2, IFI27, OAS1, SIGLEC1, ISG15, SLC1A2, | 0.868 | 0.857 | 0.880 | 0.942 | 0.898 | 0.986 | 0.945 | 0.875 | 1 |
| IL1R2, OLAH, FAM20A, RETN, MMP8 | | | | | | | | | |
| RSAD2, IFI27, OAS1, IFIT1, SIGLEC1, ISG15, | 0.868 | 0.858 | 0.879 | 0.937 | 0.894 | 0.980 | 0.951 | 0.882 | 1 |
| HERC6, SLC1A2, IL1R2, FAM20A, MMP8 | | | | | | | | | |
| RSAD2, IFI27, IFIT1, SIGLEC1, ISG15, HERC6, | 0.868 | 0.858 | 0.879 | 0.939 | 0.894 | 0.985 | 0.951 | 0.882 | 1 |
| SLC1A2, IL1R2, FAM20A, RETN, MMP8 | | | | | | | | | |
| IFI27, IFIT1, IFI44L, SIGLEC1, ISG15, HERC6, | 0.868 | 0.858 | 0.879 | 0.939 | 0.895 | 0.984 | 0.952 | 0.883 | 1 |
| SLC1A2, IL1R2, FAM20A, RETN, MMP8 | | | | | | | | | |
| IFI27, OAS1, IFI44L, SIGLEC1, ISG15, HERC6, | 0.868 | 0.857 | 0.879 | 0.938 | 0.893 | 0.984 | 0.947 | 0.874 | 1 |
| SLC1A2, IL1R2, FAM20A, RETN, MMP8 | | | | | | | | | |
| RSAD2, IFI27, OAS1, IFIT1, IFI44L, SIGLEC1, | 0.868 | 0.856 | 0.880 | 0.931 | 0.875 | 0.987 | 0.952 | 0.895 | 1 |
| ISG15, OLAH, FAM20A, RETN, MMP8 | | | | | | | | | |
| RSAD2, IFI27, OAS1, IFIT1, SIGLEC1, HERC6, | 0.868 | 0.857 | 0.879 | 0.941 | 0.897 | 0.984 | 0.946 | 0.872 | 1 |
| SLC1A2, IL1R2, FAM20A, RETN, MMP8 | | | | | | | | | |
| RSAD2, IFI27, IFIT1, IFI44L, SIGLEC1, ISG15, | 0.868 | 0.858 | 0.878 | 0.939 | 0.895 | 0.984 | 0.934 | 0.865 | 1 |
| HERC6, SLC1A2, IL1R2, OLAH, FAM20A | | | | | | | | | |
| IFI27, OAS1, IFIT1, IFI44L, SIGLEC1, ISG15, | 0.868 | 0.856 | 0.880 | 0.938 | 0.895 | 0.981 | 0.951 | 0.882 | 1 |
| HERC6, SLC1A2, IL1R2, FAM20A, MMP8 | | | | | | | | | |
| IFI27, IFIT1, IFI44L, SIGLEC1, HERC6, SLC1A2, | 0.868 | 0.857 | 0.879 | 0.940 | 0.894 | 0.986 | 0.953 | 0.888 | 1 |
| IL1R2, OLAH, FAM20A, RETN, MMP8 | | | | | | | | | |
| RSAD2, IFI27, OAS1, IFI44L, SIGLEC1, HERC6, | 0.868 | 0.857 | 0.879 | 0.938 | 0.890 | 0.986 | 0.947 | 0.883 | 1 |
| SLC1A2, OLAH, FAM20A, RETN, MMP8 | | | | | | | | | |
| RSAD2, IFI27, OAS1, SIGLEC1, ISG15, HERC6, | 0.868 | 0.856 | 0.880 | 0.942 | 0.896 | 0.987 | 0.942 | 0.876 | 1 |
| SLC1A2, OLAH, FAM20A, RETN, MMP8 | | | | | | | | | |
| RSAD2, IFI27, IFIT1, IFI44L, SIGLEC1, ISG15, | 0.868 | 0.858 | 0.878 | 0.938 | 0.895 | 0.981 | 0.952 | 0.883 | 1 |
| HERC6, SLC1A2, IL1R2, FAM20A, MMP8 | | | | | | | | | |
| IFI27, OAS1, IFIT1, IFI44L, SIGLEC1, ISG15, | 0.868 | 0.857 | 0.879 | 0.939 | 0.895 | 0.982 | 0.949 | 0.879 | 1 |
| SLC1A2, IL1R2, FAM20A, RETN, MMP8 | | | | | | | | | |
| RSAD2, IFI27, OAS1, IFIT1, IFI44L, SIGLEC1, | 0.868 | 0.857 | 0.879 | 0.936 | 0.889 | 0.983 | 0.937 | 0.870 | 1 |
| HERC6, SLC1A2, OLAH, FAM20A, RETN | | | | | | | | | |
| RSAD2, IFI27, OAS1, IFIT1, SIGLEC1, HERC6, | 0.868 | 0.857 | 0.878 | 0.938 | 0.892 | 0.984 | 0.935 | 0.866 | 1 |
| SLC1A2, IL1R2, OLAH, FAM20A, RETN | | | | | | | | | |
| IFI27, OAS1, IFIT1, IFI44L, SIGLEC1, ISG15, | 0.868 | 0.856 | 0.880 | 0.937 | 0.888 | 0.986 | 0.947 | 0.883 | 1 |
| SLC1A2, OLAH, FAM20A, RETN, MMP8 | | | | | | | | | |
| RSAD2, IFI27, OAS1, IFIT1, SIGLEC1, ISG15, | 0.868 | 0.857 | 0.878 | 0.938 | 0.892 | 0.984 | 0.936 | 0.867 | 1 |
| HERC6, SLC1A2, OLAH, FAM20A, RETN | | | | | | | | | |
| RSAD2, IFI27, OAS1, IFI44L, SIGLEC1, ISG15, | 0.868 | 0.857 | 0.878 | 0.938 | 0.892 | 0.983 | 0.938 | 0.869 | 1 |
| HERC6, SLC1A2, IL1R2, OLAH, FAM20A | | | | | | | | | |
| RSAD2, IFI27, OAS1, IFIT1, SIGLEC1, SLC1A2, | 0.867 | 0.856 | 0.879 | 0.937 | 0.889 | 0.984 | 0.951 | 0.888 | 1 |
| IL1R2, OLAH, FAM20A, RETN, MMP8 | | | | | | | | | |
| RSAD2, IFI27, OAS1, IFIT1, SIGLEC1, ISG15, | 0.867 | 0.856 | 0.879 | 0.937 | 0.887 | 0.986 | 0.947 | 0.883 | 1 |
| SLC1A2, OLAH, FAM20A, RETN, MMP8 | | | | | | | | | |
| RSAD2, IFI27, OAS1, IFIT1, IFI44L, SIGLEC1, | 0.867 | 0.857 | 0.877 | 0.936 | 0.888 | 0.983 | 0.941 | 0.875 | 1 |
| ISG15, SLC1A2, IL1R2, OLAH, FAM20A | | | | | | | | | |
| RSAD2, IFI27, IFIT1, IFI44L, SIGLEC1, HERC6, | 0.867 | 0.855 | 0.879 | 0.941 | 0.894 | 0.987 | 0.949 | 0.887 | 1 |
| SLC1A2, OLAH, FAM20A, RETN, MMP8 | | | | | | | | | |
| RSAD2, IFI27, OAS1, IFI44L, SIGLEC1, ISG15, | 0.867 | 0.857 | 0.878 | 0.938 | 0.895 | 0.981 | 0.949 | 0.879 | 1 |
| HERC6, SLC1A2, IL1R2, FAM20A, MMP8 | | | | | | | | | |
| RSAD2, IFI27, IFIT1, IFI44L, ISG15, HERC6, | 0.867 | 0.855 | 0.879 | 0.933 | 0.880 | 0.986 | 0.932 | 0.859 | 1 |
| IL1R2, OLAH, FAM20A, RETN, MMP8 | | | | | | | | | |
| RSAD2, IFI27, IFI44L, SIGLEC1, HERC6, | 0.867 | 0.855 | 0.879 | 0.939 | 0.893 | 0.986 | 0.948 | 0.882 | 1 |
| SLC1A2, IL1R2, OLAH, FAM20A, RETN, MMP8 | | | | | | | | | |
| RSAD2, IFI27, OAS1, IFIT1, IFI44L, SIGLEC1, | 0.867 | 0.857 | 0.876 | 0.927 | 0.873 | 0.980 | 0.953 | 0.903 | 1 |
| HERC6, IL1R2, OLAH, RETN, MMP8 | | | | | | | | | |
| RSAD2, IFI27, OAS1, IFIT1, HERC6, SLC1A2, | 0.867 | 0.856 | 0.877 | 0.935 | 0.887 | 0.983 | 0.921 | 0.839 | 1 |
| IL1R2, OLAH, FAM20A, RETN, MMP8 | | | | | | | | | |
| IFI27, OAS1, IFIT1, IFI44L, SIGLEC1, ISG15, | 0.867 | 0.856 | 0.878 | 0.935 | 0.885 | 0.984 | 0.941 | 0.872 | 1 |
| HERC6, SLC1A2, FAM20A, RETN, MMP8 | | | | | | | | | |
| RSAD2, IFI27, IFIT1, IFI44L, SIGLEC1, HERC6, | 0.867 | 0.856 | 0.878 | 0.938 | 0.892 | 0.985 | 0.939 | 0.873 | 1 |
| SLC1A2, IL1R2, OLAH, FAM20A, RETN | | | | | | | | | |
| IFI27, OAS1, IFIT1, IFI44L, SIGLEC1, ISG15, | 0.867 | 0.856 | 0.877 | 0.936 | 0.891 | 0.981 | 0.937 | 0.865 | 1 |
| HERC6, SLC1A2, IL1R2, FAM20A, RETN | | | | | | | | | |
| RSAD2, IFI27, OAS1, SIGLEC1, ISG15, HERC6, | 0.867 | 0.856 | 0.878 | 0.939 | 0.894 | 0.983 | 0.949 | 0.878 | 1 |
| SLC1A2, IL1R2, FAM20A, RETN, MMP8 | | | | | | | | | |
| RSAD2, IFI27, OAS1, IFIT1, SIGLEC1, ISG15, | 0.867 | 0.856 | 0.877 | 0.935 | 0.887 | 0.984 | 0.941 | 0.875 | 1 |
| SLC1A2, IL1R2, OLAH, FAM20A, RETN | | | | | | | | | |
| IFI27, OAS1, IFI44L, SIGLEC1, ISG15, HERC6, | 0.867 | 0.856 | 0.877 | 0.936 | 0.890 | 0.982 | 0.936 | 0.866 | 1 |
| SLC1A2, IL1R2, OLAH, FAM20A, RETN | | | | | | | | | |
| RSAD2, IFI27, OAS1, IFIT1, IFI44L, SIGLEC1, | 0.867 | 0.855 | 0.878 | 0.935 | 0.884 | 0.986 | 0.952 | 0.885 | 1 |
| ISG15, IL1R2, FAM20A, RETN, MMP8 | | | | | | | | | |
| IFI27, IFIT1, IFI44L, SIGLEC1, ISG15, HERC6, | 0.867 | 0.856 | 0.877 | 0.939 | 0.895 | 0.984 | 0.938 | 0.872 | 1 |
| SLC1A2, IL1R2, OLAH, FAM20A, RETN | | | | | | | | | |
| RSAD2, IFI27, IFIT1, IFI44L, SIGLEC1, ISG15, | 0.866 | 0.854 | 0.878 | 0.938 | 0.889 | 0.986 | 0.948 | 0.885 | 1 |
| SLC1A2, OLAH, FAM20A, RETN, MMP8 | | | | | | | | | |
| RSAD2, IFI27, OAS1, IFIT1, IFI44L, SIGLEC1, | 0.866 | 0.857 | 0.876 | 0.938 | 0.895 | 0.980 | 0.943 | 0.874 | 1 |
| ISG15, HERC6, SLC1A2, IL1R2, FAM20A | | | | | | | | | |
| RSAD2, IFI27, OAS1, IFIT1, IFI44L, SIGLEC1, | 0.866 | 0.856 | 0.877 | 0.937 | 0.895 | 0.980 | 0.951 | 0.882 | 1 |
| ISG15, SLC1A2, IL1R2, FAM20A, MMP8 | | | | | | | | | |
| RSAD2, IFI27, IFIT1, ISG15, HERC6, SLC1A2, | 0.866 | 0.856 | 0.876 | 0.933 | 0.885 | 0.982 | 0.923 | 0.843 | 1 |
| IL1R2, OLAH, FAM20A, RETN, MMP8 | | | | | | | | | |
| IFI27, OAS1, IFI44L, ISG15, HERC6, SLC1A2, | 0.866 | 0.855 | 0.877 | 0.936 | 0.889 | 0.984 | 0.924 | 0.843 | 1 |
| IL1R2, OLAH, FAM20A, RETN, MMP8 | | | | | | | | | |
| RSAD2, IFI27, OAS1, IFIT1, IFI44L, ISG 15, | 0.866 | 0.855 | 0.877 | 0.931 | 0.879 | 0.983 | 0.926 | 0.852 | 1 |
| HERC6, IL1R2, OLAH, FAM20A, RETN | | | | | | | | | |
| IFI27, OAS1, IFIT1, IFI44L, ISG15, HERC6, | 0.866 | 0.855 | 0.878 | 0.934 | 0.885 | 0.984 | 0.922 | 0.840 | 1 |
| SLC1A2, OLAH, FAM20A, RETN, MMP8 | | | | | | | | | |
| RSAD2, IFI27, OAS1, IFIT1, IFI44L, HERC6, | 0.866 | 0.855 | 0.878 | 0.934 | 0.886 | 0.983 | 0.924 | 0.844 | 1 |
| SLC1A2, IL1R2, OLAH, FAM20A, MMP8 | | | | | | | | | |
| IFI27, OAS1, IFIT1, IFI44L, SIGLEC1, ISG15, | 0.866 | 0.855 | 0.877 | 0.935 | 0.886 | 0.985 | 0.936 | 0.867 | 1 |
| SLC1A2, IL1R2, OLAH, FAM20A, RETN | | | | | | | | | |
| RSAD2, IFI27, OAS1, IFI44L, SIGLEC1, HERC6, | 0.866 | 0.855 | 0.877 | 0.939 | 0.894 | 0.984 | 0.947 | 0.876 | 1 |
| SLC1A2, IL1R2, FAM20A, RETN, MMP8 | | | | | | | | | |
| RSAD2, IFI27, OAS1, IFIT1, ISG15, HERC6, | 0.866 | 0.855 | 0.877 | 0.934 | 0.886 | 0.981 | 0.922 | 0.840 | 1 |
| SLC1A2, IL1R2, OLAH, FAM20A, MMP8 | | | | | | | | | |
| RSAD2, IFI27, IFIT1, IFI44L, SIGLEC1, ISG15, | 0.866 | 0.855 | 0.877 | 0.938 | 0.893 | 0.984 | 0.935 | 0.867 | 1 |
| HERC6, SLC1A2, OLAH, FAM20A, RETN | | | | | | | | | |
| RSAD2, IFI27, IFIT1, IFI44L, SIGLEC1, ISG15, | 0.866 | 0.856 | 0.876 | 0.939 | 0.894 | 0.984 | 0.949 | 0.879 | 1 |
| SLC1A2, IL1R2, FAM20A, RETN, MMP8 | | | | | | | | | |
| IFI27, OAS1, IFIT1, ISG15, HERC6, SLC1A2, | 0.866 | 0.855 | 0.877 | 0.933 | 0.885 | 0.982 | 0.921 | 0.839 | 1 |
| IL1R2, OLAH, FAM20A, RETN, MMP8 | | | | | | | | | |
| RSAD2, IFI27, OAS1, IFIT1, IFI44L, SIGLEC1, | 0.866 | 0.854 | 0.878 | 0.934 | 0.883 | 0.985 | 0.950 | 0.889 | 1 |
| SLC1A2, OLAH, FAM20A, RETN, MMP8 | | | | | | | | | |
| RSAD2, IFI27, OAS1, IFIT1, IFI44L, ISG15, | 0.866 | 0.854 | 0.878 | 0.927 | 0.871 | 0.982 | 0.934 | 0.865 | 1 |
| IL1R2, OLAH, FAM20A, RETN, MMP8 | | | | | | | | | |
| RSAD2, IFI27, IFIT1, SIGLEC1, ISG15, HERC6, | 0.866 | 0.855 | 0.877 | 0.939 | 0.894 | 0.984 | 0.934 | 0.866 | 1 |
| SLC1A2, IL1R2, OLAH, FAM20A, RETN | | | | | | | | | |
| RSAD2, IFI27, OAS1, IFI44L, ISG15, HERC6, | 0.866 | 0.854 | 0.878 | 0.937 | 0.891 | 0.984 | 0.922 | 0.841 | 1 |
| SLC1A2, IL1R2, OLAH, FAM20A, MMP8 | | | | | | | | | |
| RSAD2, IFI27, OAS1, IFIT1, SIGLEC1, ISG15, | 0.866 | 0.856 | 0.876 | 0.927 | 0.874 | 0.981 | 0.958 | 0.909 | 1 |
| HERC6, IL1R2, OLAH, RETN, MMP8 | | | | | | | | | |
| RSAD2, IFI27, OAS1, IFIT1, IFI44L, ISG15, | 0.866 | 0.854 | 0.878 | 0.931 | 0.881 | 0.982 | 0.923 | 0.844 | 1 |
| HERC6, SLC1A2, OLAH, FAM20A, MMP8 | | | | | | | | | |
| RSAD2, IFI27, OAS1, IFIT1, IFI44L, SIGLEC1, | 0.866 | 0.856 | 0.875 | 0.927 | 0.874 | 0.981 | 0.950 | 0.895 | 1 |
| ISG15, HERC6, OLAH, RETN, MMP8 | | | | | | | | | |
| IFI27, OAS1, IFIT1, IFI44L, HERC6, SLC1A2, | 0.866 | 0.855 | 0.877 | 0.937 | 0.889 | 0.984 | 0.923 | 0.841 | 1 |
| IL1R2, OLAH, FAM20A, RETN, MMP8 | | | | | | | | | |
| RSAD2, IFI27, OAS1, IFIT1, IFI44L, SIGLEC1, | 0.866 | 0.855 | 0.876 | 0.933 | 0.881 | 0.984 | 0.938 | 0.872 | 1 |
| ISG15, SLC1A2, OLAH, FAM20A, RETN | | | | | | | | | |
| RSAD2, IFI27, OAS1, IFIT1, SIGLEC1, ISG15, | 0.866 | 0.856 | 0.876 | 0.935 | 0.885 | 0.985 | 0.940 | 0.869 | 1 |
| HERC6, SLC1A2, FAM20A, RETN, MMP8 | | | | | | | | | |
| RSAD2, IFI27, IFI44L, SIGLEC1, ISG15, SLC1A2, | 0.866 | 0.853 | 0.878 | 0.944 | 0.901 | 0.986 | 0.946 | 0.878 | 1 |
| IL1R2, OLAH, FAM20A, RETN, MMP8 | | | | | | | | | |
| RSAD2, IFI27, OAS1, IFIT1, ISG15, HERC6, | 0.866 | 0.855 | 0.876 | 0.933 | 0.883 | 0.983 | 0.923 | 0.842 | 1 |
| SLC1A2, OLAH, FAM20A, RETN, MMP8 | | | | | | | | | |
| IFI27, OAS1, IFIT1, IFI44L, SIGLEC1, ISG15, | 0.866 | 0.856 | 0.875 | 0.927 | 0.875 | 0.980 | 0.952 | 0.901 | 1 |
| HERC6, IL1R2, OLAH, RETN, MMP8 | | | | | | | | | |
| RSAD2, IFI27, OAS1, IFI44L, SIGLEC1, ISG15, | 0.866 | 0.855 | 0.876 | 0.936 | 0.890 | 0.983 | 0.937 | 0.866 | 1 |
| HERC6, SLC1A2, OLAH, FAM20A, RETN | | | | | | | | | |
| RSAD2, IFI27, OAS1, IFIT1, SIGLEC1, ISG15, | 0.865 | 0.855 | 0.876 | 0.938 | 0.894 | 0.982 | 0.949 | 0.879 | 1 |
| SLC1A2, IL1R2, FAM20A, RETN, MMP8 | | | | | | | | | |
| RSAD2, IFI27, OAS1, SIGLEC1, ISG15, HERC6, | 0.865 | 0.854 | 0.876 | 0.936 | 0.889 | 0.983 | 0.935 | 0.864 | 1 |
| SLC1A2, IL1R2, OLAH, FAM20A, RETN | | | | | | | | | |
| RSAD2, IFI27, IFI44L, SIGLEC1, ISG15, HERC6, | 0.865 | 0.855 | 0.876 | 0.937 | 0.891 | 0.983 | 0.936 | 0.867 | 1 |
| SLC1A2, IL1R2, OLAH, FAM20A, RETN | | | | | | | | | |
| RSAD2, IFI27, IFI44L, SIGLEC1, ISG15, HERC6, | 0.865 | 0.853 | 0.877 | 0.942 | 0.896 | 0.988 | 0.943 | 0.876 | 1 |
| SLC1A2, OLAH, FAM20A, RETN, MMP8 | | | | | | | | | |
| RSAD2, IFI27, OAS1, IFIT1, IFI44L, SIGLEC1, | 0.865 | 0.854 | 0.876 | 0.934 | 0.885 | 0.984 | 0.941 | 0.872 | 1 |
| HERC6, SLC1A2, FAM20A, RETN, MMP8 | | | | | | | | | |
| RSAD2, IFI27, IFIT1, IFI44L, SIGLEC1, ISG15, | 0.865 | 0.855 | 0.875 | 0.937 | 0.889 | 0.985 | 0.936 | 0.868 | 1 |
| SLC1A2, IL1R2, OLAH, FAM20A, RETN | | | | | | | | | |
| RSAD2, IFI27, IFIT1, IFI44L, ISG15, HERC6, | 0.865 | 0.854 | 0.876 | 0.937 | 0.891 | 0.983 | 0.924 | 0.847 | 1 |
| SLC1A2, IL1R2, OLAH, FAM20A, MMP8 | | | | | | | | | |
| RSAD2, IFI27, OAS1, IFIT1, IFI44L, SIGLEC1, | 0.865 | 0.856 | 0.874 | 0.926 | 0.872 | 0.979 | 0.941 | 0.882 | 1 |
| ISG15, HERC6, IL1R2, OLAH, RETN | | | | | | | | | |
| RSAD2, IFI27, OAS1, IFI44L, SIGLEC1, ISG15, | 0.865 | 0.856 | 0.875 | 0.926 | 0.873 | 0.980 | 0.952 | 0.901 | 1 |
| HERC6, IL1R2, OLAH, RETN, MMP8 | | | | | | | | | |
| RSAD2, IFI27, IFI44L, SIGLEC1, ISG15, HERC6, | 0.865 | 0.854 | 0.876 | 0.938 | 0.892 | 0.984 | 0.949 | 0.879 | 1 |
| SLC1A2, IL1R2, FAM20A, RETN, MMP8 | | | | | | | | | |
| RSAD2, IFI27, OAS1, IFIT1, SIGLEC1, ISG15, | 0.865 | 0.854 | 0.875 | 0.937 | 0.893 | 0.981 | 0.937 | 0.864 | 1 |
| HERC6, SLC1A2, IL1R2, FAM20A, RETN | | | | | | | | | |
| RSAD2, IFI27, OAS1, IFIT1, IFI44L, SIGLEC1, | 0.865 | 0.855 | 0.875 | 0.933 | 0.884 | 0.982 | 0.934 | 0.860 | 1 |
| ISG15, HERC6, SLC1A2, FAM20A, RETN | | | | | | | | | |
| RSAD2, IFI27, OAS1, IFI44L, SIGLEC1, SLC1A2, | 0.865 | 0.853 | 0.876 | 0.936 | 0.888 | 0.984 | 0.948 | 0.883 | 1 |
| IL1R2, OLAH, FAM20A, RETN, MMP8 | | | | | | | | | |
| RSAD2, IFI27, OAS1, IFIT1, IFI44L, ISG15, | 0.865 | 0.854 | 0.876 | 0.934 | 0.885 | 0.982 | 0.925 | 0.848 | 1 |
| SLC1A2, IL1R2, OLAH, FAM20A, MMP8 | | | | | | | | | |
| RSAD2, IFI27, OAS1, IFIT1, IFI44L, ISG15, | 0.865 | 0.854 | 0.875 | 0.935 | 0.887 | 0.982 | 0.920 | 0.837 | 1 |
| HERC6, SLC1A2, IL1R2, OLAH, FAM20A | | | | | | | | | |
| RSAD2, IFI27, OAS1, IFI44L, SIGLEC1, ISG15, | 0.865 | 0.854 | 0.875 | 0.935 | 0.888 | 0.983 | 0.943 | 0.875 | 1 |
| SLC1A2, IL1R2, OLAH, FAM20A, RETN | | | | | | | | | |
| RSAD2, IFI27, IFIT1, IFI44L, SIGLEC1, SLC1A2, | 0.865 | 0.853 | 0.877 | 0.938 | 0.892 | 0.985 | 0.950 | 0.886 | 1 |
| IL1R2, OLAH, FAM20A, RETN, MMP8 | | | | | | | | | |
| RSAD2, IFI27, OAS1, IFIT1, IFI44L, SIGLEC1, | 0.865 | 0.854 | 0.876 | 0.936 | 0.891 | 0.981 | 0.939 | 0.866 | 1 |
| HERC6, SLC1A2, IL1R2, FAM20A, RETN | | | | | | | | | |
| IFI27, OAS1, IFIT1, IFI44L, ISG15, HERC6, | 0.865 | 0.853 | 0.876 | 0.935 | 0.887 | 0.983 | 0.924 | 0.845 | 1 |
| SLC1A2, IL1R2, OLAH, FAM20A, MMP8 | | | | | | | | | |
| RSAD2, IFI27, OAS1, IFIT1, IFI44L, SIGLEC1, | 0.865 | 0.853 | 0.876 | 0.938 | 0.894 | 0.983 | 0.948 | 0.876 | 1 |
| SLC1A2, IL1R2, FAM20A, RETN, MMP8 | | | | | | | | | |
| RSAD2, IFI27, OAS1, IFIT1, ISG15, SLC1A2, | 0.864 | 0.854 | 0.875 | 0.933 | 0.884 | 0.982 | 0.925 | 0.847 | 1 |
| IL1R2, OLAH, FAM20A, RETN, MMP8 | | | | | | | | | |
| RSAD2, IFI27, IFIT1, IFI44L, ISG15, SLC1A2, | 0.864 | 0.854 | 0.875 | 0.935 | 0.887 | 0.983 | 0.924 | 0.845 | 1 |
| IL1R2, OLAH, FAM20A, RETN, MMP8 | | | | | | | | | |
| IFI27, OAS1, IFIT1, IFI44L, SIGLEC1, SLC1A2, | 0.864 | 0.852 | 0.877 | 0.937 | 0.889 | 0.984 | 0.951 | 0.887 | 1 |
| IL1R2, OLAH, FAM20A, RETN, MMP8 | | | | | | | | | |
| RSAD2, IFI27, OAS1, IFIT1, IFI44L, HERC6, | 0.864 | 0.852 | 0.876 | 0.936 | 0.888 | 0.985 | 0.923 | 0.842 | 1 |
| SLC1A2, OLAH, FAM20A, RETN, MMP8 | | | | | | | | | |
| RSAD2, IFI27, OAS1, IFI44L, SIGLEC1, ISG15, | 0.864 | 0.853 | 0.875 | 0.939 | 0.894 | 0.983 | 0.947 | 0.876 | 1 |
| SLC1A2, IL1R2, FAM20A, RETN, MMP8 | | | | | | | | | |
| RSAD2, IFI27, IFI44L, ISG15, HERC6, SLC1A2, | 0.864 | 0.852 | 0.875 | 0.937 | 0.889 | 0.984 | 0.922 | 0.841 | 1 |
| IL1R2, OLAH, FAM20A, RETN, MMP8 | | | | | | | | | |
| RSAD2, IFI27, IFIT1, IFI44L, HERC6, SLC1A2, | 0.864 | 0.853 | 0.875 | 0.937 | 0.889 | 0.984 | 0.924 | 0.844 | 1 |
| IL1R2, OLAH, FAM20A, RETN, MMP8 | | | | | | | | | |
| RSAD2, IFI27, OAS1, IFIT1, IFI44L, SIGLEC1, | 0.864 | 0.852 | 0.875 | 0.933 | 0.882 | 0.984 | 0.941 | 0.876 | 1 |
| SLC1A2, IL1R2, OLAH, FAM20A, RETN | | | | | | | | | |
| RSAD2, IFI27, OAS1, ISG15, HERC6, SLC1A2, | 0.864 | 0.852 | 0.875 | 0.935 | 0.888 | 0.983 | 0.923 | 0.840 | 1 |
| IL1R2, OLAH, FAM20A, RETN, MMP8 | | | | | | | | | |
| RSAD2, IFI27, OAS1, IFI44L, ISG15, HERC6, | 0.864 | 0.852 | 0.875 | 0.934 | 0.885 | 0.984 | 0.923 | 0.842 | 1 |
| SLC1A2, OLAH, FAM20A, RETN, MMP8 | | | | | | | | | |
| RSAD2, IFI27, OAS1, IFIT1, IFI44L, ISG15, | 0.863 | 0.852 | 0.875 | 0.932 | 0.880 | 0.984 | 0.928 | 0.847 | 1 |
| HERC6, IL1R2, FAM20A, RETN, MMP8 | | | | | | | | | |
| IFI27, OAS1, IFIT1, IFI44L, ISG15, SLC1A2, | 0.863 | 0.852 | 0.874 | 0.933 | 0.884 | 0.982 | 0.923 | 0.843 | 1 |
| IL1R2, OLAH, FAM20A, RETN, MMP8 | | | | | | | | | |
| RSAD2, IFI27, IFIT1, IFI44L, SIGLEC1, ISG15, | 0.863 | 0.854 | 0.873 | 0.927 | 0.875 | 0.980 | 0.950 | 0.898 | 1 |
| HERC6, IL1R2, OLAH, RETN, MMP8 | | | | | | | | | |
| RSAD2, IFI27, IFIT1, IFI44L, SIGLEC1, ISG15, | 0.863 | 0.853 | 0.874 | 0.931 | 0.879 | 0.984 | 0.942 | 0.868 | 1 |
| HERC6, SLC1A2, FAM20A, RETN, MMP8 | | | | | | | | | |
| IFI27, IFIT1, IFI44L, ISG15, HERC6, SLC1A2, | 0.863 | 0.852 | 0.874 | 0.934 | 0.887 | 0.982 | 0.923 | 0.843 | 1 |
| IL1R2, OLAH, FAM20A, RETN, MMP8 | | | | | | | | | |
| RSAD2, IFI27, OAS1, IFI44L, HERC6, SLC1A2, | 0.863 | 0.851 | 0.876 | 0.938 | 0.891 | 0.985 | 0.924 | 0.844 | 1 |
| IL1R2, OLAH, FAM20A, RETN, MMP8 | | | | | | | | | |
| RSAD2, IFI27, IFIT1, IFI44L, ISG15, HERC6, | 0.863 | 0.852 | 0.875 | 0.936 | 0.888 | 0.985 | 0.923 | 0.843 | 1 |
| SLC1A2, OLAH, FAM20A, RETN, MMP8 | | | | | | | | | |
| RSAD2, IFI27, OAS1, IFI44L, SIGLEC1, HERC6, | 0.863 | 0.852 | 0.875 | 0.935 | 0.888 | 0.983 | 0.938 | 0.869 | 1 |
| SLC1A2, IL1R2, OLAH, FAM20A, RETN | | | | | | | | | |
| RSAD2, IFI27, OAS1, IFI44L, SIGLEC1, ISG15, | 0.863 | 0.851 | 0.876 | 0.936 | 0.886 | 0.985 | 0.947 | 0.885 | 1 |
| SLC1A2, OLAH, FAM20A, RETN, MMP8 | | | | | | | | | |
| RSAD2, IFI27, IFIT1, IFI44L, SIGLEC1, ISG15, | 0.863 | 0.853 | 0.873 | 0.934 | 0.888 | 0.980 | 0.946 | 0.874 | 1 |
| HERC6, SLC1A2, IL1R2, FAM20A, RETN | | | | | | | | | |
| OAS1, IFIT1, IFI44L, SIGLEC1, ISG15, HERC6, | 0.863 | 0.853 | 0.873 | 0.924 | 0.869 | 0.980 | 0.963 | 0.909 | 1 |
| IL1R2, OLAH, FAM20A, RETN, MMP8 | | | | | | | | | |
| RSAD2, IFI27, OAS1, IFIT1, IFI44L, ISG15, | 0.863 | 0.852 | 0.874 | 0.934 | 0.886 | 0.983 | 0.920 | 0.835 | 1 |
| HERC6, SLC1A2, OLAH, FAM20A, RETN | | | | | | | | | |
| RSAD2, IFI27, OAS1, IFIT1, IFI44L, HERC6, | 0.863 | 0.852 | 0.873 | 0.934 | 0.886 | 0.982 | 0.922 | 0.839 | 1 |
| SLC1A2, IL1R2, OLAH, FAM20A, RETN | | | | | | | | | |
| RSAD2, OAS1, IFIT1, SIGLEC1, ISG15, HERC6, | 0.863 | 0.853 | 0.872 | 0.923 | 0.867 | 0.979 | 0.963 | 0.909 | 1 |
| IL1R2, OLAH, FAM20A, RETN, MMP8 | | | | | | | | | |
| RSAD2, OAS1, IFIT1, IFI44L, SIGLEC1, ISG15, | 0.862 | 0.852 | 0.873 | 0.923 | 0.868 | 0.977 | 0.963 | 0.909 | 1 |
| HERC6, IL1R2, OLAH, FAM20A, MMP8 | | | | | | | | | |
| RSAD2, IFI27, OAS1, IFIT1, IFI44L, ISG15, | 0.862 | 0.852 | 0.872 | 0.923 | 0.867 | 0.979 | 0.949 | 0.894 | 1 |
| HERC6, IL1R2, OLAH, RETN, MMP8 | | | | | | | | | |
| RSAD2, IFI27, OAS1, IFIT1, SIGLEC1, HERC6, | 0.862 | 0.852 | 0.872 | 0.932 | 0.883 | 0.982 | 0.950 | 0.892 | 1 |
| SLC1A2, IL1R2, OLAH, RETN, MMP8 | | | | | | | | | |
| IFI27, OAS1, IFIT1, IFI44L, SIGLEC1, HERC6, | 0.862 | 0.852 | 0.872 | 0.931 | 0.883 | 0.980 | 0.949 | 0.891 | 1 |
| SLC1A2, IL1R2, OLAH, RETN, MMP8 | | | | | | | | | |
| RSAD2, IFI27, OAS1, IFIT1, IFI44L, SIGLEC1, | 0.862 | 0.853 | 0.871 | 0.927 | 0.879 | 0.976 | 0.948 | 0.898 | 0.998 |
| ISG15, HERC6, IL1R2, OLAH, MMP8 | | | | | | | | | |
| RSAD2, IFI27, OAS1, IFIT1, IFI44L, ISG15, | 0.862 | 0.850 | 0.873 | 0.930 | 0.877 | 0.983 | 0.923 | 0.845 | 1 |
| SLC1A2, OLAH, FAM20A, RETN, MMP8 | | | | | | | | | |
| IFI27, OAS1, IFIT1, IFI44L, ISG15, HERC6, | 0.862 | 0.851 | 0.873 | 0.934 | 0.887 | 0.982 | 0.918 | 0.835 | 1 |
| SLC1A2, IL1R2, OLAH, FAM20A, RETN | | | | | | | | | |
| RSAD2, IFI27, OAS1, IFI44L, SIGLEC1, ISG15, | 0.862 | 0.851 | 0.873 | 0.935 | 0.890 | 0.980 | 0.939 | 0.864 | 1 |
| HERC6, SLC1A2, IL1R2, FAM20A, RETN | | | | | | | | | |
| RSAD2, OAS1, IFIT1, IFI44L, SIGLEC1, HERC6, | 0.862 | 0.851 | 0.872 | 0.925 | 0.871 | 0.980 | 0.962 | 0.907 | 1 |
| IL1R2, OLAH, FAM20A, RETN, MMP8 | | | | | | | | | |
| RSAD2, IFI27, OAS1, IFI44L, ISG15, HERC6, | 0.862 | 0.851 | 0.873 | 0.934 | 0.886 | 0.982 | 0.917 | 0.833 | 1 |
| SLC1A2, IL1R2, OLAH, FAM20A, RETN | | | | | | | | | |
| RSAD2, IFI27, OAS1, IFIT1, IFI44L, SIGLEC1, | 0.862 | 0.852 | 0.871 | 0.926 | 0.874 | 0.979 | 0.955 | 0.905 | 1 |
| ISG15, HERC6, IL1R2, RETN, MMP8 | | | | | | | | | |
| RSAD2, IFI27, OAS1, IFIT1, IFI44L, SIGLEC1, | 0.862 | 0.852 | 0.871 | 0.931 | 0.882 | 0.981 | 0.945 | 0.882 | 1 |
| HERC6, SLC1A2, IL1R2, OLAH, RETN | | | | | | | | | |
| RSAD2, IFI27, OAS1, IFI44L, ISG15, SLC1A2, | 0.861 | 0.850 | 0.873 | 0.935 | 0.887 | 0.984 | 0.924 | 0.845 | 1 |
| IL1R2, OLAH, FAM20A, RETN, MMP8 | | | | | | | | | |
| RSAD2, IFI27, OAS1, IFIT1, IFI44L, SIGLEC1, | 0.861 | 0.851 | 0.872 | 0.933 | 0.887 | 0.980 | 0.942 | 0.873 | 1 |
| ISG15, SLC1A2, IL1R2, FAM20A, RETN | | | | | | | | | |
| IFI27, OAS1, IFIT1, SIGLEC1, ISG15, HERC6, | 0.861 | 0.850 | 0.872 | 0.931 | 0.882 | 0.980 | 0.947 | 0.886 | 1 |
| SLC1A2, IL1R2, OLAH, RETN, MMP8 | | | | | | | | | |
| RSAD2, IFI27, OAS1, IFIT1, ISG15, HERC6, | 0.861 | 0.851 | 0.872 | 0.934 | 0.886 | 0.981 | 0.916 | 0.829 | 1 |
| SLC1A2, IL1R2, OLAH, FAM20A, RETN | | | | | | | | | |
| RSAD2, IFI27, OAS1, IFIT1, IFI44L, SIGLEC1, | 0.861 | 0.851 | 0.871 | 0.932 | 0.883 | 0.981 | 0.949 | 0.890 | 1 |
| HERC6, SLC1A2, OLAH, RETN, MMP8 | | | | | | | | | |
| RSAD2, OAS1, IFIT1, IFI44L, SIGLEC1, ISG15, | 0.861 | 0.850 | 0.871 | 0.923 | 0.868 | 0.979 | 0.964 | 0.909 | 1 |
| HERC6, IL1R2, FAM20A, RETN, MMP8 | | | | | | | | | |
| RSAD2, IFI27, OAS1, IFIT1, SIGLEC1, ISG15, | 0.861 | 0.851 | 0.870 | 0.931 | 0.882 | 0.981 | 0.946 | 0.885 | 1 |
| HERC6, SLC1A2, IL1R2, OLAH, RETN | | | | | | | | | |
| RSAD2, IFI27, OAS1, IFIT1, IFI44L, SLC1A2, | 0.860 | 0.849 | 0.872 | 0.934 | 0.884 | 0.983 | 0.926 | 0.850 | 1 |
| IL1R2, OLAH, FAM20A, RETN, MMP8 | | | | | | | | | |
| RSAD2, IFI27, OAS1, IFI44L, SIGLEC1, ISG15, | 0.860 | 0.849 | 0.872 | 0.931 | 0.877 | 0.984 | 0.942 | 0.868 | 1 |
| HERC6, SLC1A2, FAM20A, RETN, MMP8 | | | | | | | | | |
| RSAD2, IFIT1, IFI44L, SIGLEC1, ISG15, HERC6, | 0.860 | 0.850 | 0.870 | 0.924 | 0.869 | 0.980 | 0.962 | 0.907 | 1 |
| IL1R2, OLAH, FAM20A, RETN, MMP8 | | | | | | | | | |
| RSAD2, IFI27, IFIT1, IFI44L, ISG15, HERC6, | 0.860 | 0.849 | 0.871 | 0.936 | 0.888 | 0.983 | 0.918 | 0.837 | 1 |
| SLC1A2, IL1R2, OLAH, FAM20A, RETN | | | | | | | | | |
| RSAD2, IFI27, OAS1, IFI44L, SIGLEC1, HERC6, | 0.860 | 0.849 | 0.870 | 0.931 | 0.882 | 0.980 | 0.950 | 0.892 | 1 |
| SLC1A2, IL1R2, OLAH, RETN, MMP8 | | | | | | | | | |
| RSAD2, OAS1, IFIT1, IFI44L, SIGLEC1, HERC6, | 0.860 | 0.849 | 0.871 | 0.930 | 0.883 | 0.976 | 0.954 | 0.892 | 1 |
| SLC1A2, IL1R2, OLAH, FAM20A, MMP8 | | | | | | | | | |
| RSAD2, IFI27, IFIT1, SIGLEC1, ISG15, HERC6, | 0.860 | 0.850 | 0.870 | 0.931 | 0.883 | 0.980 | 0.950 | 0.897 | 1 |
| SLC1A2, IL1R2, OLAH, RETN, MMP8 | | | | | | | | | |
| RSAD2, IFI27, OAS1, IFIT1, IFI44L, SIGLEC1, | 0.860 | 0.850 | 0.869 | 0.924 | 0.870 | 0.978 | 0.952 | 0.897 | 1 |
| ISG15, IL1R2, OLAH, RETN, MMP8 | | | | | | | | | |
| RSAD2, IFI27, OAS1, IFI44L, SIGLEC1, ISG15, | 0.860 | 0.850 | 0.869 | 0.929 | 0.879 | 0.979 | 0.943 | 0.881 | 1 |
| HERC6, SLC1A2, IL1R2, OLAH, RETN | | | | | | | | | |
| IFI27, OAS1, IFIT1, IFI44L, ISG15, HERC6, | 0.860 | 0.849 | 0.870 | 0.933 | 0.884 | 0.982 | 0.927 | 0.843 | 1 |
| SLC1A2, IL1R2, FAM20A, RETN, MMP8 | | | | | | | | | |
| IFI27, OAS1, IFIT1, IFI44L, SIGLEC1, ISG15, | 0.860 | 0.849 | 0.870 | 0.931 | 0.882 | 0.981 | 0.946 | 0.884 | 1 |
| HERC6, SLC1A2, OLAH, RETN, MMP8 | | | | | | | | | |
| IFI27, OAS1, IFI44L, SIGLEC1, ISG15, HERC6, | 0.860 | 0.850 | 0.870 | 0.933 | 0.884 | 0.981 | 0.947 | 0.887 | 1 |
| SLC1A2, IL1R2, OLAH, RETN, MMP8 | | | | | | | | | |
| RSAD2, IFIT1, IFI44L, SIGLEC1, ISG15, HERC6, | 0.860 | 0.849 | 0.870 | 0.929 | 0.883 | 0.976 | 0.957 | 0.893 | 1 |
| SLC1A2, IL1R2, OLAH, FAM20A, MMP8 | | | | | | | | | |
| OAS1, IFIT1, SIGLEC1, ISG15, HERC6, SLC1A2, | 0.859 | 0.849 | 0.869 | 0.929 | 0.882 | 0.976 | 0.957 | 0.891 | 1 |
| IL1R2, OLAH, FAM20A, RETN, MMP8 | | | | | | | | | |
| RSAD2, OAS1, IFIT1, SIGLEC1, ISG15, HERC6, | 0.859 | 0.849 | 0.870 | 0.928 | 0.881 | 0.975 | 0.961 | 0.899 | 1 |
| SLC1A2, IL1R2, OLAH, FAM20A, MMP8 | | | | | | | | | |
| RSAD2, IFI27, OAS1, IFIT1, IFI44L, ISG15, | 0.859 | 0.849 | 0.870 | 0.931 | 0.880 | 0.982 | 0.921 | 0.839 | 1 |
| SLC1A2, IL1R2, OLAH, FAM20A, RETN | | | | | | | | | |
| IFI27, OAS1, IFIT1, IFI44L, SIGLEC1, ISG15, | 0.859 | 0.849 | 0.869 | 0.930 | 0.880 | 0.979 | 0.937 | 0.873 | 1 |
| HERC6, SLC1A2, IL1R2, OLAH, RETN | | | | | | | | | |
| RSAD2, IFI27, IFIT1, IFI44L, SIGLEC1, HERC6, | 0.859 | 0.849 | 0.869 | 0.930 | 0.881 | 0.980 | 0.952 | 0.901 | 1 |
| SLC1A2, IL1R2, OLAH, RETN, MMP8 | | | | | | | | | |
| OAS1, IFIT1, IFI44L, SIGLEC1, ISG15, HERC6, | 0.859 | 0.848 | 0.870 | 0.929 | 0.882 | 0.976 | 0.954 | 0.890 | 1 |
| SLC1A2, IL1R2, OLAH, FAM20A, MMP8 | | | | | | | | | |
| IFIT1, IFI44L, SIGLEC1, ISG15, HERC6, SLC1A2, | 0.859 | 0.849 | 0.869 | 0.930 | 0.884 | 0.977 | 0.959 | 0.899 | 1 |
| IL1R2, OLAH, FAM20A, RETN, MMP8 | | | | | | | | | |
| RSAD2, OAS1, IFIT1, IFI44L, SIGLEC1, ISG15, | 0.859 | 0.849 | 0.869 | 0.922 | 0.865 | 0.979 | 0.951 | 0.893 | 1 |
| HERC6, OLAH, FAM20A, RETN, MMP8 | | | | | | | | | |
| RSAD2, OAS1, IFIT1, IFI44L, SIGLEC1, ISG15, | 0.859 | 0.848 | 0.870 | 0.929 | 0.880 | 0.978 | 0.935 | 0.863 | 1 |
| HERC6, SLC1A2, OLAH, FAM20A, MMP8 | | | | | | | | | |
| OAS1, IFIT1, IFI44L, SIGLEC1, ISG15, HERC6, | 0.859 | 0.849 | 0.869 | 0.930 | 0.881 | 0.979 | 0.940 | 0.873 | 1 |
| SLC1A2, OLAH, FAM20A, RETN, MMP8 | | | | | | | | | |
| RSAD2, IFI27, OAS1, IFIT1, IFI44L, SIGLEC1, | 0.859 | 0.849 | 0.869 | 0.927 | 0.876 | 0.978 | 0.935 | 0.859 | 1 |
| ISG15, HERC6, SLC1A2, FAM20A, MMP8 | | | | | | | | | |
| RSAD2, IFI27, OAS1, IFIT1, IFI44L, HERC6, | 0.859 | 0.849 | 0.869 | 0.927 | 0.876 | 0.979 | 0.938 | 0.872 | 1 |
| SLC1A2, IL1R2, OLAH, RETN, MMP8 | | | | | | | | | |
| RSAD2, IFI27, OAS1, IFIT1, IFI44L, SIGLEC1, | 0.859 | 0.849 | 0.869 | 0.931 | 0.881 | 0.981 | 0.945 | 0.883 | 1 |
| ISG15, HERC6, SLC1A2, OLAH, RETN | | | | | | | | | |
| OAS1, IFIT1, IFI44L, SIGLEC1, ISG15, HERC6, | 0.859 | 0.849 | 0.869 | 0.929 | 0.882 | 0.975 | 0.958 | 0.892 | 1 |
| SLC1A2, IL1R2, FAM20A, RETN, MMP8 | | | | | | | | | |
| OAS1, IFIT1, IFI44L, SIGLEC1, HERC6, SLC1A2, | 0.858 | 0.848 | 0.869 | 0.929 | 0.882 | 0.977 | 0.950 | 0.886 | 1 |
| IL1R2, OLAH, FAM20A, RETN, MMP8 | | | | | | | | | |
| RSAD2, IFI27, OAS1, SIGLEC1, ISG15, HERC6, | 0.858 | 0.847 | 0.870 | 0.931 | 0.881 | 0.980 | 0.948 | 0.888 | 1 |
| SLC1A2, IL1R2, OLAH, RETN, MMP8 | | | | | | | | | |
| RSAD2, IFI27, OAS1, IFIT1, ISG15, HERC6, | 0.858 | 0.848 | 0.869 | 0.928 | 0.877 | 0.979 | 0.939 | 0.874 | 1 |
| SLC1A2, IL1R2, OLAH, RETN, MMP8 | | | | | | | | | |
| IFI27, IFIT1, IFI44L, SIGLEC1, ISG15, HERC6, | 0.858 | 0.848 | 0.868 | 0.930 | 0.882 | 0.979 | 0.949 | 0.896 | 1 |
| SLC1A2, IL1R2, OLAH, RETN, MMP8 | | | | | | | | | |
| RSAD2, IFI27, OAS1, IFIT1, IFI44L, HERC6, | 0.858 | 0.848 | 0.869 | 0.932 | 0.883 | 0.981 | 0.927 | 0.843 | 1 |
| SLC1A2, IL1R2, FAM20A, RETN, MMP8 | | | | | | | | | |
| RSAD2, OAS1, IFIT1, SIGLEC1, HERC6, | 0.858 | 0.848 | 0.869 | 0.929 | 0.882 | 0.976 | 0.959 | 0.895 | 1 |
| SLC1A2, IL1R2, OLAH, FAM20A, RETN, MMP8 | | | | | | | | | |
| RSAD2, IFI27, OAS1, IFIT1, SIGLEC1, ISG15, | 0.858 | 0.847 | 0.869 | 0.932 | 0.882 | 0.981 | 0.949 | 0.890 | 1 |
| HERC6, SLC1A2, OLAH, RETN, MMP8 | | | | | | | | | |
| RSAD2, OAS1, IFIT1, IFI44L, SIGLEC1, ISG15, | 0.858 | 0.848 | 0.868 | 0.927 | 0.879 | 0.975 | 0.960 | 0.902 | 1 |
| SLC1A2, IL1R2, OLAH, FAM20A, MMP8 | | | | | | | | | |
| RSAD2, IFI27, OAS1, IFIT1, IFI44L, SIGLEC1, | 0.858 | 0.848 | 0.868 | 0.929 | 0.881 | 0.978 | 0.946 | 0.890 | 1 |
| ISG15, HERC6, SLC1A2, IL1R2, OLAH | | | | | | | | | |
| RSAD2, OAS1, IFIT1, SIGLEC1, ISG15, HERC6, | 0.858 | 0.848 | 0.868 | 0.926 | 0.876 | 0.977 | 0.948 | 0.881 | 1 |
| SLC1A2, OLAH, FAM20A, RETN, MMP8 | | | | | | | | | |
| RSAD2, OAS1, IFIT1, IFI44L, SIGLEC1, HERC6, | 0.858 | 0.847 | 0.869 | 0.929 | 0.880 | 0.978 | 0.941 | 0.875 | 1 |
| SLC1A2, OLAH, FAM20A, RETN, MMP8 | | | | | | | | | |
| RSAD2, IFI27, OAS1, IFI44L, SIGLEC1, ISG15, | 0.858 | 0.847 | 0.869 | 0.931 | 0.882 | 0.981 | 0.950 | 0.892 | 1 |
| HERC6, SLC1A2, OLAH, RETN, MMP8 | | | | | | | | | |
| RSAD2, IFIT1, IFI44L, SIGLEC1, ISG15, HERC6, | 0.858 | 0.848 | 0.868 | 0.930 | 0.881 | 0.978 | 0.938 | 0.868 | 1 |
| SLC1A2, OLAH, FAM20A, RETN, MMP8 | | | | | | | | | |
| RSAD2, IFI27, OAS1, IFIT1, IFI44L, SIGLEC1, | 0.858 | 0.847 | 0.869 | 0.927 | 0.872 | 0.982 | 0.941 | 0.867 | 1 |
| ISG15, SLC1A2, FAM20A, RETN, MMP8 | | | | | | | | | |
| RSAD2, IFI27, OAS1, IFIT1, IFI44L, SIGLEC1, | 0.858 | 0.849 | 0.867 | 0.924 | 0.870 | 0.978 | 0.941 | 0.878 | 1 |
| ISG15, HERC6, SLC1A2, RETN, MMP8 | | | | | | | | | |
| RSAD2, IFIT1, SIGLEC1, ISG15, HERC6, | 0.858 | 0.848 | 0.868 | 0.929 | 0.881 | 0.976 | 0.959 | 0.893 | 1 |
| SLC1A2, IL1R2, OLAH, FAM20A, RETN, MMP8 | | | | | | | | | |
| RSAD2, IFI27, OAS1, IFIT1, IFI44L, SIGLEC1, | 0.858 | 0.848 | 0.868 | 0.928 | 0.880 | 0.976 | 0.950 | 0.897 | 1 |
| HERC6, SLC1A2, IL1R2, OLAH, MMP8 | | | | | | | | | |
| RSAD2, IFI27, OAS1, IFIT1, IFI44L, SIGLEC1, | 0.857 | 0.848 | 0.867 | 0.929 | 0.880 | 0.978 | 0.948 | 0.893 | 1 |
| ISG15, SLC1A2, IL1R2, OLAH, MMP8 | | | | | | | | | |
| RSAD2, IFIT1, IFI44L, SIGLEC1, HERC6, | 0.857 | 0.847 | 0.868 | 0.93 | 0.883 | 0.977 | 0.953 | 0.892 | 1 |
| SLC1A2, IL1R2, OLAH, FAM20A, RETN, MMP8 | | | | | | | | | |
| RSAD2, OAS1, IFIT1, IFI44L, SIGLEC1, ISG15, | 0.857 | 0.847 | 0.868 | 0.92 | 0.863 | 0.978 | 0.968 | 0.918 | 1 |
| IL1R2, OLAH, FAM20A, RETN, MMP8 | | | | | | | | | |
| IFI27, OAS1, IFIT1, IFI44L, SIGLEC1, ISG15, | 0.857 | 0.848 | 0.867 | 0.926 | 0.879 | 0.974 | 0.954 | 0.905 | 1 |
| HERC6, SLC1A2, IL1R2, RETN, MMP8 | | | | | | | | | |
| RSAD2, IFI27, OAS1, IFIT1, IFI44L, SIGLEC1, | 0.857 | 0.848 | 0.866 | 0.928 | 0.880 | 0.976 | 0.945 | 0.882 | 1 |
| ISG15, HERC6, SLC1A2, IL1R2, RETN | | | | | | | | | |
| RSAD2, OAS1, IFI44L, SIGLEC1, ISG15, HERC6, | 0.857 | 0.846 | 0.868 | 0.917 | 0.859 | 0.975 | 0.960 | 0.900 | 1 |
| IL1R2, OLAH, FAM20A, RETN, MMP8 | | | | | | | | | |
| RSAD2, IFI27, OAS1, IFIT1, IFI44L, SIGLEC1, | 0.857 | 0.847 | 0.867 | 0.927 | 0.876 | 0.978 | 0.952 | 0.895 | 1 |
| SLC1A2, IL1R2, OLAH, RETN, MMP8 | | | | | | | | | |
| RSAD2, IFI27, OAS1, IFI44L, ISG15, HERC6, | 0.857 | 0.847 | 0.867 | 0.931 | 0.881 | 0.981 | 0.940 | 0.875 | 1 |
| SLC1A2, IL1R2, OLAH, RETN, MMP8 | | | | | | | | | |
| RSAD2, IFI27, IFI44L, SIGLEC1, ISG15, HERC6, | 0.857 | 0.847 | 0.867 | 0.931 | 0.883 | 0.980 | 0.949 | 0.896 | 1 |
| SLC1A2, IL1R2, OLAH, RETN, MMP8 | | | | | | | | | |
| RSAD2, OAS1, SIGLEC1, ISG15, HERC6, | 0.857 | 0.847 | 0.867 | 0.925 | 0.877 | 0.974 | 0.957 | 0.892 | 1 |
| SLC1A2, IL1R2, OLAH, FAM20A, RETN, MMP8 | | | | | | | | | |
| RSAD2, IFI27, OAS1, IFIT1, ISG15, HERC6, | 0.857 | 0.846 | 0.867 | 0.933 | 0.884 | 0.981 | 0.926 | 0.843 | 1 |
| SLC1A2, IL1R2, FAM20A, RETN, MMP8 | | | | | | | | | |
| IFI27, OAS1, IFIT1, IFI44L, SIGLEC1, ISG15, | 0.857 | 0.846 | 0.867 | 0.929 | 0.881 | 0.977 | 0.941 | 0.885 | 0.998 |
| HERC6, SLC1A2, IL1R2, OLAH, MMP8 | | | | | | | | | |
| RSAD2, OAS1, IFIT1, IFI44L, SIGLEC1, ISG15, | 0.857 | 0.847 | 0.867 | 0.927 | 0.879 | 0.975 | 0.938 | 0.871 | 1 |
| HERC6, SLC1A2, IL1R2, OLAH, FAM20A | | | | | | | | | |
| RSAD2, IFI27, OAS1, IFIT1, SIGLEC1, ISG15, | 0.857 | 0.846 | 0.868 | 0.929 | 0.880 | 0.978 | 0.942 | 0.884 | 1 |
| HERC6, SLC1A2, IL1R2, OLAH, MMP8 | | | | | | | | | |
| OAS1, IFIT1, IFI44L, SIGLEC1, ISG15, SLC1A2, | 0.857 | 0.846 | 0.867 | 0.927 | 0.878 | 0.975 | 0.958 | 0.898 | 1 |
| IL1R2, OLAH, FAM20A, RETN, MMP8 | | | | | | | | | |
| RSAD2, OAS1, IFI44L, SIGLEC1, ISG15, HERC6, | 0.857 | 0.846 | 0.867 | 0.922 | 0.872 | 0.972 | 0.959 | 0.897 | 1 |
| SLC1A2, IL1R2, OLAH, FAM20A, MMP8 | | | | | | | | | |
| IFI27, OAS1, IFIT1, IFI44L, ISG15, HERC6, | 0.857 | 0.846 | 0.867 | 0.929 | 0.879 | 0.980 | 0.939 | 0.874 | 1 |
| SLC1A2, IL1R2, OLAH, RETN, MMP8 | | | | | | | | | |
| RSAD2, IFIT1, IFI44L, SIGLEC1, ISG15, HERC6, | 0.857 | 0.847 | 0.866 | 0.929 | 0.882 | 0.975 | 0.957 | 0.889 | 1 |
| SLC1A2, IL1R2, FAM20A, RETN, MMP8 | | | | | | | | | |
| RSAD2, IFI27, OAS1, IFIT1, IFI44L, SIGLEC1, | 0.857 | 0.847 | 0.866 | 0.929 | 0.881 | 0.977 | 0.950 | 0.892 | 1 |
| HERC6, SLC1A2, IL1R2, RETN, MMP8 | | | | | | | | | |
| IFI27, OAS1, IFIT1, IFI44L, SIGLEC1, ISG15, | 0.856 | 0.847 | 0.866 | 0.928 | 0.878 | 0.978 | 0.950 | 0.894 | 1 |
| SLC1A2, IL1R2, OLAH, RETN, MMP8 | | | | | | | | | |
| RSAD2, IFI27, OAS1, IFIT1, IFI44L, SIGLEC1, | 0.856 | 0.845 | 0.868 | 0.931 | 0.881 | 0.980 | 0.942 | 0.885 | 1 |
| ISG15, HERC6, SLC1A2, OLAH, MMP8 | | | | | | | | | |
| RSAD2, IFI27, OAS1, IFIT1, IFI44L, ISG15, | 0.856 | 0.846 | 0.867 | 0.929 | 0.877 | 0.980 | 0.940 | 0.876 | 1 |
| HERC6, SLC1A2, OLAH, RETN, MMP8 | | | | | | | | | |
| RSAD2, IFI27, IFIT1, IFI44L, SIGLEC1, ISG15, | 0.856 | 0.846 | 0.867 | 0.931 | 0.881 | 0.980 | 0.951 | 0.899 | 1 |
| HERC6, SLC1A2, OLAH, RETN, MMP8 | | | | | | | | | |
| RSAD2, OAS1, IFIT1, IFI44L, SIGLEC1, ISG15, | 0.856 | 0.846 | 0.866 | 0.918 | 0.861 | 0.976 | 0.953 | 0.898 | 1 |
| HERC6, IL1R2, OLAH, FAM20A, RETN | | | | | | | | | |
| RSAD2, IFI27, OAS1, IFIT1, SIGLEC1, ISG15, | 0.856 | 0.846 | 0.866 | 0.931 | 0.881 | 0.980 | 0.951 | 0.892 | 1 |
| SLC1A2, IL1R2, OLAH, RETN, MMP8 | | | | | | | | | |
| RSAD2, IFI27, IFIT1, IFI44L, ISG15, HERC6, | 0.856 | 0.846 | 0.867 | 0.934 | 0.886 | 0.982 | 0.930 | 0.849 | 1 |
| SLC1A2, IL1R2, FAM20A, RETN, MMP8 | | | | | | | | | |
| RSAD2, IFI27, OAS1, IFIT1, SIGLEC1, ISG15, | 0.856 | 0.847 | 0.866 | 0.930 | 0.882 | 0.977 | 0.953 | 0.899 | 1 |
| HERC6, SLC1A2, IL1R2, RETN, MMP8 | | | | | | | | | |
| RSAD2, IFI27, OAS1, IFI44L, SIGLEC1, ISG15, | 0.856 | 0.845 | 0.867 | 0.930 | 0.881 | 0.978 | 0.945 | 0.890 | 1 |
| HERC6, SLC1A2, IL1R2, OLAH, MMP8 | | | | | | | | | |
| RSAD2, OAS1, IFIT1, SIGLEC1, ISG15, HERC6, | 0.856 | 0.846 | 0.866 | 0.928 | 0.881 | 0.975 | 0.960 | 0.897 | 1 |
| SLC1A2, IL1R2, FAM20A, RETN, MMP8 | | | | | | | | | |
| RSAD2, IFI27, IFIT1, IFI44L, SIGLEC1, ISG15, | 0.856 | 0.846 | 0.866 | 0.928 | 0.878 | 0.978 | 0.941 | 0.884 | 0.998 |
| HERC6, SLC1A2, IL1R2, OLAH, RETN | | | | | | | | | |
| RSAD2, OAS1, IFIT1, IFI44L, SIGLEC1, HERC6, | 0.856 | 0.845 | 0.866 | 0.928 | 0.881 | 0.975 | 0.958 | 0.892 | 1 |
| SLC1A2, IL1R2, FAM20A, RETN, MMP8 | | | | | | | | | |
| RSAD2, OAS1, IFIT1, IFI44L, SIGLEC1, ISG15, | 0.856 | 0.845 | 0.867 | 0.927 | 0.882 | 0.973 | 0.961 | 0.899 | 1 |
| HERC6, SLC1A2, IL1R2, FAM20A, MMP8 | | | | | | | | | |
| RSAD2, IFI27, OAS1, IFI44L, ISG15, HERC6, | 0.856 | 0.844 | 0.867 | 0.933 | 0.885 | 0.982 | 0.923 | 0.834 | 1 |
| SLC1A2, IL1R2, FAM20A, RETN, MMP8 | | | | | | | | | |
| RSAD2, IFI27, IFIT1, IFI44L, ISG15, HERC6, | 0.856 | 0.846 | 0.866 | 0.929 | 0.878 | 0.979 | 0.937 | 0.875 | 0.999 |
| SLC1A2, IL1R2, OLAH, RETN, MMP8 | | | | | | | | | |
| RSAD2, IFI27, OAS1, IFIT1, IFI44L, SIGLEC1, | 0.856 | 0.846 | 0.865 | 0.930 | 0.880 | 0.980 | 0.947 | 0.883 | 1 |
| ISG15, SLC1A2, IL1R2, OLAH, RETN | | | | | | | | | |
| RSAD2, IFI27, OAS1, IFIT1, IFI44L, ISG15, | 0.856 | 0.845 | 0.866 | 0.929 | 0.878 | 0.979 | 0.933 | 0.861 | 1 |
| HERC6, SLC1A2, IL1R2, OLAH, RETN | | | | | | | | | |
| RSAD2, IFI27, OAS1, IFIT1, IFI44L, ISG15, | 0.856 | 0.844 | 0.867 | 0.931 | 0.885 | 0.977 | 0.926 | 0.840 | 1 |
| HERC6, SLC1A2, IL1R2, FAM20A, MMP8 | | | | | | | | | |
| RSAD2, IFI27, OAS1, IFI44L, SIGLEC1, ISG15, | 0.855 | 0.846 | 0.865 | 0.929 | 0.881 | 0.977 | 0.949 | 0.890 | 1 |
| HERC6, SLC1A2, IL1R2, RETN, MMP8 | | | | | | | | | |
| RSAD2, IFI27, OAS1, IFIT1, IFI44L, ISG15, | 0.855 | 0.845 | 0.866 | 0.933 | 0.886 | 0.980 | 0.926 | 0.840 | 1 |
| HERC6, SLC1A2, IL1R2, FAM20A, RETN | | | | | | | | | |
| RSAD2, IFIT1, IFI44L, SIGLEC1, ISG15, SLC1A2, | 0.855 | 0.845 | 0.865 | 0.926 | 0.878 | 0.975 | 0.959 | 0.899 | 1 |
| IL1R2, OLAH, FAM20A, RETN, MMP8 | | | | | | | | | |
| RSAD2, OAS1, IFIT1, IFI44L, SIGLEC1, ISG15, | 0.855 | 0.845 | 0.865 | 0.926 | 0.876 | 0.975 | 0.933 | 0.863 | 1 |
| HERC6, SLC1A2, OLAH, FAM20A, RETN | | | | | | | | | |
| RSAD2, OAS1, IFIT1, SIGLEC1, ISG15, SLC1A2, | 0.855 | 0.845 | 0.865 | 0.927 | 0.879 | 0.975 | 0.963 | 0.906 | 1 |
| IL1R2, OLAH, FAM20A, RETN, MMP8 | | | | | | | | | |
| RSAD2, OAS1, IFIT1, IFI44L, SIGLEC1, ISG15, | 0.855 | 0.845 | 0.865 | 0.922 | 0.870 | 0.975 | 0.945 | 0.883 | 1 |
| SLC1A2, OLAH, FAM20A, RETN, MMP8 | | | | | | | | | |
| RSAD2, IFI27, IFIT1, IFI44L, SIGLEC1, ISG15, | 0.855 | 0.844 | 0.865 | 0.930 | 0.882 | 0.979 | 0.953 | 0.902 | 1 |
| SLC1A2, IL1R2, OLAH, RETN, MMP8 | | | | | | | | | |
| RSAD2, OAS1, IFIT1, SIGLEC1, ISG15, HERC6, | 0.855 | 0.845 | 0.865 | 0.926 | 0.877 | 0.975 | 0.948 | 0.884 | 1 |
| SLC1A2, IL1R2, OLAH, FAM20A, RETN | | | | | | | | | |
| OAS1, IFIT1, IFI44L, SIGLEC1, ISG15, HERC6, | 0.855 | 0.844 | 0.865 | 0.927 | 0.878 | 0.975 | 0.934 | 0.865 | 1 |
| SLC1A2, IL1R2, OLAH, FAM20A, RETN | | | | | | | | | |
| OAS1, IFI44L, SIGLEC1, ISG15, HERC6, | 0.855 | 0.844 | 0.865 | 0.923 | 0.874 | 0.973 | 0.959 | 0.897 | 1 |
| SLC1A2, IL1R2, OLAH, FAM20A, RETN, MMP8 | | | | | | | | | |
| RSAD2, IFI27, IFIT1, IFI44L, SIGLEC1, ISG15, | 0.855 | 0.845 | 0.864 | 0.929 | 0.879 | 0.978 | 0.945 | 0.892 | 0.997 |
| HERC6, SLC1A2, IL1R2, OLAH, MMP8 | | | | | | | | | |
| RSAD2, IFI27, OAS1, IFIT1, IFI44L, ISG 15, | 0.854 | 0.843 | 0.866 | 0.924 | 0.869 | 0.980 | 0.930 | 0.849 | 1 |
| HERC6, SLC1A2, FAM20A, RETN, MMP8 | | | | | | | | | |
| RSAD2, IFI27, OAS1, IFIT1, IFI44L, ISG 15, | 0.854 | 0.843 | 0.865 | 0.931 | 0.882 | 0.979 | 0.927 | 0.844 | 1 |
| SLC1A2, IL1R2, FAM20A, RETN, MMP8 | | | | | | | | | |
| RSAD2, IFI27, OAS1, IFIT1, IFI44L, ISG15, | 0.854 | 0.844 | 0.864 | 0.926 | 0.875 | 0.978 | 0.941 | 0.878 | 1 |
| SLC1A2, IL1R2, OLAH, RETN, MMP8 | | | | | | | | | |
| RSAD2, IFI27, OAS1, IFIT1, IFI44L, SIGLEC1, | 0.854 | 0.843 | 0.864 | 0.928 | 0.875 | 0.981 | 0.953 | 0.895 | 1 |
| ISG15, SLC1A2, OLAH, RETN, MMP8 | | | | | | | | | |
| RSAD2, OAS1, IFIT1, IFI44L, SIGLEC1, SLC1A2, | 0.854 | 0.843 | 0.864 | 0.927 | 0.878 | 0.975 | 0.959 | 0.904 | 1 |
| IL1R2, OLAH, FAM20A, RETN, MMP8 | | | | | | | | | |
| RSAD2, IFI44L, SIGLEC1, ISG15, HERC6, | 0.854 | 0.843 | 0.864 | 0.924 | 0.876 | 0.973 | 0.959 | 0.897 | 1 |
| SLC1A2, IL1R2, OLAH, FAM20A, RETN, MMP8 | | | | | | | | | |
| RSAD2, OAS1, IFIT1, IFI44L, SIGLEC1, HERC6, | 0.854 | 0.843 | 0.864 | 0.927 | 0.879 | 0.975 | 0.937 | 0.869 | 1 |
| SLC1A2, IL1R2, OLAH, FAM20A, RETN | | | | | | | | | |
| RSAD2, IFI27, OAS1, IFI44L, SIGLEC1, ISG15, | 0.853 | 0.843 | 0.864 | 0.931 | 0.882 | 0.980 | 0.953 | 0.896 | 1 |
| SLC1A2, IL1R2, OLAH, RETN, MMP8 | | | | | | | | | |
| RSAD2, OAS1, IFIT1, IFI44L, SIGLEC1, ISG15, | 0.853 | 0.843 | 0.864 | 0.926 | 0.876 | 0.977 | 0.939 | 0.868 | 1 |
| HERC6, SLC1A2, FAM20A, RETN, MMP8 | | | | | | | | | |
| RSAD2, IFI27, OAS1, IFIT1, IFI44L, ISG15, | 0.853 | 0.843 | 0.864 | 0.927 | 0.879 | 0.976 | 0.935 | 0.871 | 0.998 |
| HERC6, SLC1A2, IL1R2, OLAH, MMP8 | | | | | | | | | |
| RSAD2, OAS1, IFI44L, SIGLEC1, HERC6, | 0.853 | 0.842 | 0.864 | 0.924 | 0.875 | 0.973 | 0.958 | 0.897 | 1 |
| SLC1A2, IL1R2, OLAH, FAM20A, RETN, MMP8 | | | | | | | | | |
| RSAD2, OAS1, IFI44L, SIGLEC1, ISG15, | 0.853 | 0.843 | 0.863 | 0.922 | 0.872 | 0.971 | 0.959 | 0.897 | 1 |
| SLC1A2, IL1R2, OLAH, FAM20A, RETN, MMP8 | | | | | | | | | |
| RSAD2, OAS1, IFIT1, IFI44L, SIGLEC1, ISG15, | 0.853 | 0.843 | 0.863 | 0.927 | 0.880 | 0.974 | 0.960 | 0.895 | 1 |
| SLC1A2, IL1R2, FAM20A, RETN, MMP8 | | | | | | | | | |
| RSAD2, IFIT1, IFI44L, SIGLEC1, ISG15, HERC6, | 0.853 | 0.843 | 0.863 | 0.926 | 0.878 | 0.975 | 0.937 | 0.867 | 1 |
| SLC1A2, IL1R2, OLAH, FAM20A, RETN | | | | | | | | | |
| RSAD2, OAS1, IFI44L, SIGLEC1, ISG15, HERC6, | 0.853 | 0.842 | 0.863 | 0.925 | 0.877 | 0.973 | 0.958 | 0.892 | 1 |
| SLC1A2, IL1R2, FAM20A, RETN, MMP8 | | | | | | | | | |
| RSAD2, OAS1, IFI44L, SIGLEC1, ISG15, HERC6, | 0.852 | 0.842 | 0.863 | 0.920 | 0.867 | 0.974 | 0.941 | 0.872 | 1 |
| SLC1A2, OLAH, FAM20A, RETN, MMP8 | | | | | | | | | |
| RSAD2, OAS1, IFIT1, IFI44L, SIGLEC1, ISG15, | 0.852 | 0.842 | 0.862 | 0.926 | 0.879 | 0.973 | 0.946 | 0.879 | 1 |
| HERC6, SLC1A2, IL1R2, FAM20A, RETN | | | | | | | | | |
| RSAD2, IFI27, OAS1, IFIT1, IFI44L, ISG15, | 0.852 | 0.842 | 0.861 | 0.923 | 0.872 | 0.974 | 0.935 | 0.866 | 1 |
| HERC6, SLC1A2, IL1R2, RETN, MMP8 | | | | | | | | | |
| RSAD2, IFI27, IFIT1, IFI44L, SIGLEC1, ISG15, | 0.851 | 0.842 | 0.861 | 0.927 | 0.880 | 0.975 | 0.954 | 0.908 | 1 |
| HERC6, SLC1A2, IL1R2, RETN, MMP8 | | | | | | | | | |
| RSAD2, IFI27, OAS1, IFIT1, IFI44L, SIGLEC1, | 0.851 | 0.842 | 0.860 | 0.928 | 0.880 | 0.976 | 0.958 | 0.905 | 1 |
| ISG15, SLC1A2, IL1R2, RETN, MMP8 | | | | | | | | | |
| RSAD2, OAS1, IFIT1, IFI44L, SIGLEC1, ISG15, | 0.851 | 0.841 | 0.861 | 0.921 | 0.869 | 0.973 | 0.949 | 0.888 | 1 |
| SLC1A2, IL1R2, OLAH, FAM20A, RETN | | | | | | | | | |

Performance obtained for the identification of the viral or bacterial nature of the infection on the basis of the target 12-gene combinations:

TABLE 16

| | AUC | | | AUC | CI 95% | | AUC | CI 95% | |
|---|---|---|---|---|---|---|---|---|---|
| Model | (model) | CI 95% | | (Train) | (Train) | | (Test) | (Test) | |
| RSAD2, IFI27, OAS1, IFIT1, | 0.870 | 0.858 | 0.881 | 0.938 | 0.886 | 0.989 | 0.951 | 0.888 | 1 |
| SIGLEC1, ISG15, HERC6, IL1R2, | | | | | | | | | |
| OLAH, FAM20A, RETN, MMP8 | | | | | | | | | |
| IFI27, OAS1, IFIT1, IFI44L, | 0.870 | 0.859 | 0.881 | 0.939 | 0.888 | 0.990 | 0.952 | 0.890 | 1 |
| SIGLEC1, ISG15, HERC6, IL1R2, | | | | | | | | | |
| OLAH, FAM20A, RETN, MMP8 | | | | | | | | | |
| RSAD2, IFI27, OAS1, IFIT1, | 0.869 | 0.857 | 0.880 | 0.938 | 0.890 | 0.986 | 0.947 | 0.882 | 1 |
| IFI44L, SIGLEC1, ISG15, | | | | | | | | | |
| HERC6, IL1R2, OLAH, | | | | | | | | | |
| FAM20A, MMP8 | | | | | | | | | |
| RSAD2, IFI27, OAS1, IFIT1, | 0.868 | 0.856 | 0.880 | 0.938 | 0.887 | 0.989 | 0.951 | 0.888 | 1 |
| IFI44L, SIGLEC1, HERC6, | | | | | | | | | |
| IL1R2, OLAH, FAM20A, RETN, | | | | | | | | | |
| MMP8 | | | | | | | | | |
| RSAD2, IFI27, OAS1, IFIT1, | 0.867 | 0.855 | 0.878 | 0.943 | 0.900 | 0.986 | 0.948 | 0.881 | 1 |
| SIGLEC1, ISG15, HERC6, | | | | | | | | | |
| SLC1A2, IL1R2, OLAH, | | | | | | | | | |
| FAM20A, MMP8 | | | | | | | | | |
| RSAD2, IFI27, OAS1, IFIT1, | 0.865 | 0.855 | 0.876 | 0.932 | 0.879 | 0.985 | 0.940 | 0.876 | 1 |
| IFI44L, SIGLEC1, ISG15, | | | | | | | | | |
| HERC6, IL1R2, OLAH, | | | | | | | | | |
| FAM20A, RETN | | | | | | | | | |
| IFI27, OAS1, IFIT1, IFI44L, | 0.865 | 0.853 | 0.876 | 0.942 | 0.898 | 0.985 | 0.947 | 0.879 | 1 |
| SIGLEC1, ISG15, HERC6, | | | | | | | | | |
| SLC1A2, IL1R2, OLAH, | | | | | | | | | |
| FAM20A, MMP8 | | | | | | | | | |
| RSAD2, IFI27, OAS1, IFIT1, | 0.865 | 0.853 | 0.876 | 0.941 | 0.896 | 0.985 | 0.946 | 0.880 | 1 |
| IFI44L, SIGLEC1, HERC6, | | | | | | | | | |
| SLC1A2, IL1R2, OLAH, | | | | | | | | | |
| FAM20A, MMP8 | | | | | | | | | |
| RSAD2, IFI27, OAS1, IFI44L, | 0.864 | 0.852 | 0.877 | 0.938 | 0.886 | 0.989 | 0.948 | 0.883 | 1 |
| SIGLEC1, ISG15, HERC6, IL1R2, | | | | | | | | | |
| OLAH, FAM20A, RETN, MMP8 | | | | | | | | | |
| RSAD2, IFI27, OAS1, IFIT1, | 0.864 | 0.853 | 0.875 | 0.942 | 0.898 | 0.986 | 0.946 | 0.877 | 1 |
| IFI44L, SIGLEC1, ISG15, | | | | | | | | | |
| SLC1A2, IL1R2, OLAH, | | | | | | | | | |
| FAM20A, MMP8 | | | | | | | | | |
| IFI27, OAS1, IFIT1, SIGLEC1, | 0.864 | 0.853 | 0.876 | 0.944 | 0.900 | 0.987 | 0.945 | 0.876 | 1 |
| ISG15, HERC6, SLC1A2, IL1R2, | | | | | | | | | |
| OLAH, FAM20A, RETN, MMP8 | | | | | | | | | |
| RSAD2, IFI27, IFIT1, IFI44L, | 0.864 | 0.852 | 0.875 | 0.943 | 0.900 | 0.986 | 0.949 | 0.883 | 1 |
| SIGLEC1, ISG15, HERC6, | | | | | | | | | |
| SLC1A2, IL1R2, OLAH, | | | | | | | | | |
| FAM20A, MMP8 | | | | | | | | | |
| RSAD2, IFI27, OAS1, IFIT1, | 0.864 | 0.851 | 0.876 | 0.936 | 0.884 | 0.989 | 0.945 | 0.879 | 1 |
| IFI44L, SIGLEC1, ISG15, | | | | | | | | | |
| HERC6, OLAH, FAM20A, | | | | | | | | | |
| RETN, MMP8 | | | | | | | | | |
| RSAD2, IFI27, IFIT1, IFI44L, | 0.863 | 0.851 | 0.876 | 0.939 | 0.888 | 0.989 | 0.949 | 0.885 | 1 |
| SIGLEC1, ISG15, HERC6, IL1R2, | | | | | | | | | |
| OLAH, FAM20A, RETN, MMP8 | | | | | | | | | |
| RSAD2, IFI27, OAS1, IFIT1, | 0.863 | 0.852 | 0.875 | 0.942 | 0.898 | 0.986 | 0.942 | 0.874 | 1 |
| SIGLEC1, HERC6, SLC1A2, | | | | | | | | | |
| IL1R2, OLAH, FAM20A, RETN, | | | | | | | | | |
| MMP8 | | | | | | | | | |
| RSAD2, IFI27, OAS1, IFI44L, | 0.863 | 0.851 | 0.875 | 0.940 | 0.897 | 0.984 | 0.947 | 0.879 | 1 |
| SIGLEC1, ISG15, HERC6, | | | | | | | | | |
| SLC1A2, IL1R2, OLAH, | | | | | | | | | |
| FAM20A, MMP8 | | | | | | | | | |
| RSAD2, IFI27, OAS1, IFIT1, | 0.863 | 0.853 | 0.874 | 0.938 | 0.893 | 0.984 | 0.934 | 0.864 | 1 |
| IFI44L, SIGLEC1, ISG15, | | | | | | | | | |
| HERC6, SLC1A2, IL1R2, OLAH, | | | | | | | | | |
| FAM20A | | | | | | | | | |
| IFI27, OAS1, IFI44L, SIGLEC1, | 0.863 | 0.852 | 0.874 | 0.943 | 0.900 | 0.986 | 0.945 | 0.876 | 1 |
| ISG15, HERC6, SLC1A2, IL1R2, | | | | | | | | | |
| OLAH, FAM20A, RETN, MMP8 | | | | | | | | | |
| RSAD2, IFI27, OAS1, IFIT1, | 0.863 | 0.851 | 0.875 | 0.941 | 0.896 | 0.986 | 0.938 | 0.871 | 1 |
| IFI44L, SIGLEC1, ISG15, | | | | | | | | | |
| HERC6, SLC1A2, OLAH, | | | | | | | | | |
| FAM20A, MMP8 | | | | | | | | | |
| IFI27, OAS1, IFIT1, IFI44L, | 0.863 | 0.850 | 0.875 | 0.942 | 0.897 | 0.986 | 0.942 | 0.874 | 1 |
| SIGLEC1, HERC6, SLC1A2, | | | | | | | | | |
| IL1R2, OLAH, FAM20A, RETN, | | | | | | | | | |
| MMP8 | | | | | | | | | |
| RSAD2, IFI27, IFIT1, SIGLEC1, | 0.862 | 0.851 | 0.874 | 0.944 | 0.902 | 0.986 | 0.946 | 0.877 | 1 |
| ISG15, HERC6, SLC1A2, IL1R2, | | | | | | | | | |
| OLAH, FAM20A, RETN, MMP8 | | | | | | | | | |
| RSAD2, IFI27, OAS1, IFIT1, | 0.862 | 0.850 | 0.875 | 0.937 | 0.884 | 0.989 | 0.952 | 0.891 | 1 |
| IFI44L, SIGLEC1, ISG15, IL1R2, | | | | | | | | | |
| OLAH, FAM20A, RETN, MMP8 | | | | | | | | | |
| RSAD2, IFI27, OAS1, IFIT1, | 0.862 | 0.850 | 0.874 | 0.942 | 0.898 | 0.987 | 0.943 | 0.878 | 1 |
| SIGLEC1, ISG15, HERC6, | | | | | | | | | |
| SLC1A2, OLAH, FAM20A, | | | | | | | | | |
| RETN, MMP8 | | | | | | | | | |
| RSAD2, IFI27, OAS1, IFIT1, | 0.862 | 0.849 | 0.874 | 0.938 | 0.888 | 0.988 | 0.950 | 0.881 | 1 |
| IFI44L, SIGLEC1, ISG15, | | | | | | | | | |
| HERC6, IL1R2, FAM20A, RETN, | | | | | | | | | |
| MMP8 | | | | | | | | | |
| IFI27, OAS1, IFIT1, IFI44L, | 0.861 | 0.849 | 0.873 | 0.942 | 0.899 | 0.984 | 0.948 | 0.876 | 1 |
| SIGLEC1, ISG15, HERC6, | | | | | | | | | |
| SLC1A2, IL1R2, FAM20A, | | | | | | | | | |
| RETN, MMP8 | | | | | | | | | |
| RSAD2, IFI27, OAS1, IFIT1, | 0.861 | 0.850 | 0.872 | 0.939 | 0.894 | 0.983 | 0.936 | 0.867 | 1 |
| SIGLEC1, ISG15, HERC6, | | | | | | | | | |
| SLC1A2, IL1R2, OLAH, | | | | | | | | | |
| FAM20A, RETN | | | | | | | | | |
| IFI27, OAS1, IFIT1, IFI44L, | 0.861 | 0.848 | 0.874 | 0.943 | 0.898 | 0.987 | 0.943 | 0.878 | 1 |
| SIGLEC1, ISG15, HERC6, | | | | | | | | | |
| SLC1A2, OLAH, FAM20A, | | | | | | | | | |
| RETN, MMP8 | | | | | | | | | |
| RSAD2, IFI27, OAS1, IFIT1, | 0.861 | 0.848 | 0.874 | 0.940 | 0.894 | 0.986 | 0.941 | 0.874 | 1 |
| IFI44L, SIGLEC1, HERC6, | | | | | | | | | |
| SLC1A2, OLAH, FAM20A, | | | | | | | | | |
| RETN, MMP8 | | | | | | | | | |
| RSAD2, IFI27, OAS1, SIGLEC1, | 0.861 | 0.848 | 0.873 | 0.943 | 0.899 | 0.986 | 0.946 | 0.877 | 1 |
| ISG15, HERC6, SLC1A2, IL1R2, | | | | | | | | | |
| OLAH, FAM20A, RETN, MMP8 | | | | | | | | | |
| RSAD2, IFI27, OAS1, IFI44L, | 0.861 | 0.849 | 0.873 | 0.940 | 0.894 | 0.986 | 0.947 | 0.881 | 1 |
| SIGLEC1, HERC6, SLC1A2, | | | | | | | | | |
| IL1R2, OLAH, FAM20A, RETN, | | | | | | | | | |
| MMP8 | | | | | | | | | |
| RSAD2, IFI27, IFIT1, IFI44L, | 0.860 | 0.850 | 0.871 | 0.940 | 0.895 | 0.985 | 0.950 | 0.881 | 1 |
| SIGLEC1, ISG15, HERC6, | | | | | | | | | |
| SLC1A2, IL1R2, FAM20A, | | | | | | | | | |
| RETN, MMP8 | | | | | | | | | |
| RSAD2, IFI27, OAS1, IFIT1, | 0.860 | 0.849 | 0.872 | 0.937 | 0.891 | 0.983 | 0.937 | 0.870 | 1 |
| IFI44L, SIGLEC1, HERC6, | | | | | | | | | |
| SLC1A2, IL1R2, OLAH, | | | | | | | | | |
| FAM20A, RETN | | | | | | | | | |
| IFI27, OAS1, IFIT1, IFI44L, | 0.860 | 0.849 | 0.872 | 0.939 | 0.895 | 0.984 | 0.932 | 0.861 | 1 |
| SIGLEC1, ISG15, HERC6, | | | | | | | | | |
| SLC1A2, IL1R2, OLAH, | | | | | | | | | |
| FAM20A, RETN | | | | | | | | | |
| RSAD2, IFI27, OAS1, IFIT1, | 0.860 | 0.849 | 0.871 | 0.938 | 0.896 | 0.981 | 0.951 | 0.882 | 1 |
| IFI44L, SIGLEC1, ISG15, | | | | | | | | | |
| HERC6, SLC1A2, IL1R2, | | | | | | | | | |
| FAM20A, MMP8 | | | | | | | | | |
| RSAD2, IFI27, OAS1, IFIT1, | 0.860 | 0.848 | 0.872 | 0.942 | 0.897 | 0.986 | 0.947 | 0.880 | 1 |
| SIGLEC1, ISG15, SLC1A2, | | | | | | | | | |
| IL1R2, OLAH, FAM20A, RETN, | | | | | | | | | |
| MMP8 | | | | | | | | | |
| RSAD2, IFI27, OAS1, IFIT1, | 0.860 | 0.848 | 0.873 | 0.933 | 0.880 | 0.986 | 0.933 | 0.861 | 1 |
| IFI44L, ISG15, HERC6, IL1R2, | | | | | | | | | |
| OLAH, FAM20A, RETN, MMP8 | | | | | | | | | |
| RSAD2, IFI27, OAS1, IFIT1, | 0.860 | 0.849 | 0.871 | 0.939 | 0.895 | 0.983 | 0.949 | 0.877 | 1 |
| SIGLEC1, ISG15, HERC6, | | | | | | | | | |
| SLC1A2, IL1R2, FAM20A, | | | | | | | | | |
| RETN, MMP8 | | | | | | | | | |
| IFI27, IFIT1, IFI44L, SIGLEC1, | 0.860 | 0.848 | 0.872 | 0.944 | 0.901 | 0.987 | 0.948 | 0.879 | 1 |
| ISG15, HERC6, SLC1A2, IL1R2, | | | | | | | | | |
| OLAH, FAM20A, RETN, MMP8 | | | | | | | | | |
| RSAD2, IFI27, OAS1, IFIT1, | 0.860 | 0.848 | 0.872 | 0.941 | 0.898 | 0.984 | 0.946 | 0.872 | 1 |
| IFI44L, SIGLEC1, HERC6, | | | | | | | | | |
| SLC1A2, IL1R2, FAM20A, | | | | | | | | | |
| RETN, MMP8 | | | | | | | | | |
| RSAD2, IFI27, OAS1, IFIT1, | 0.859 | 0.848 | 0.871 | 0.938 | 0.893 | 0.984 | 0.937 | 0.870 | 1 |
| IFI44L, SIGLEC1, ISG15, | | | | | | | | | |
| HERC6, SLC1A2, OLAH, | | | | | | | | | |
| FAM20A, RETN | | | | | | | | | |
| RSAD2, IFI27, IFIT1, IFI44L, | 0.859 | 0.846 | 0.872 | 0.940 | 0.895 | 0.986 | 0.949 | 0.884 | 1 |
| SIGLEC1, HERC6, SLC1A2, | | | | | | | | | |
| IL1R2, OLAH, FAM20A, RETN, | | | | | | | | | |
| MMP8 | | | | | | | | | |
| RSAD2, IFI27, OAS1, IFI44L, | 0.859 | 0.848 | 0.870 | 0.939 | 0.894 | 0.983 | 0.947 | 0.874 | 1 |
| SIGLEC1, ISG15, HERC6, | | | | | | | | | |
| SLC1A2, IL1R2, FAM20A, | | | | | | | | | |
| RETN, MMP8 | | | | | | | | | |
| RSAD2, IFI27, IFIT1, IFI44L, | 0.859 | 0.846 | 0.871 | 0.944 | 0.902 | 0.986 | 0.948 | 0.882 | 1 |
| SIGLEC1, ISG15, SLC1A2, | | | | | | | | | |
| IL1R2, OLAH, FAM20A, RETN, | | | | | | | | | |
| MMP8 | | | | | | | | | |
| RSAD2, IFI27, IFIT1, IFI44L, | 0.859 | 0.848 | 0.870 | 0.939 | 0.893 | 0.984 | 0.934 | 0.866 | 1 |
| SIGLEC1, ISG15, HERC6, | | | | | | | | | |
| SLC1A2, IL1R2, OLAH, | | | | | | | | | |
| FAM20A, RETN | | | | | | | | | |
| RSAD2, IFI27, OAS1, IFI44L, | 0.858 | 0.847 | 0.869 | 0.938 | 0.893 | 0.983 | 0.937 | 0.866 | 1 |
| SIGLEC1, ISG15, HERC6, | | | | | | | | | |
| SLC1A2, IL1R2, OLAH, | | | | | | | | | |
| FAM20A, RETN | | | | | | | | | |
| RSAD2, IFI27, IFIT1, IFI44L, | 0.858 | 0.846 | 0.871 | 0.943 | 0.899 | 0.987 | 0.948 | 0.884 | 1 |
| SIGLEC1, ISG15, HERC6, | | | | | | | | | |
| SLC1A2, OLAH, FAM20A, | | | | | | | | | |
| RETN, MMP8 | | | | | | | | | |
| RSAD2, IFI27, IFI44L, SIGLEC1, | 0.858 | 0.845 | 0.871 | 0.943 | 0.900 | 0.986 | 0.947 | 0.880 | 1 |
| ISG15, HERC6, SLC1A2, IL1R2, | | | | | | | | | |
| OLAH, FAM20A, RETN, MMP8 | | | | | | | | | |
| IFI27, OAS1, IFIT1, IFI44L, | 0.858 | 0.845 | 0.872 | 0.942 | 0.899 | 0.986 | 0.947 | 0.880 | 1 |
| SIGLEC1, ISG15, SLC1A2, | | | | | | | | | |
| IL1R2, OLAH, FAM20A, RETN, | | | | | | | | | |
| MMP8 | | | | | | | | | |
| RSAD2, IFI27, OAS1, IFIT1, | 0.858 | 0.847 | 0.869 | 0.935 | 0.887 | 0.984 | 0.940 | 0.874 | 1 |
| IFI44L, SIGLEC1, ISG15, | | | | | | | | | |
| SLC1A2, IL1R2, OLAH, | | | | | | | | | |
| FAM20A, RETN | | | | | | | | | |
| RSAD2, IFI27, OAS1, IFIT1, | 0.858 | 0.848 | 0.868 | 0.927 | 0.874 | 0.979 | 0.952 | 0.901 | 1 |
| IFI44L, SIGLEC1, ISG15, | | | | | | | | | |
| HERC6, IL1R2, OLAH, RETN, | | | | | | | | | |
| MMP8 | | | | | | | | | |
| RSAD2, IFI27, OAS1, IFI44L, | 0.858 | 0.845 | 0.870 | 0.941 | 0.897 | 0.985 | 0.945 | 0.875 | 1 |
| SIGLEC1, ISG15, SLC1A2, | | | | | | | | | |
| IL1R2, OLAH, FAM20A, RETN, | | | | | | | | | |
| MMP8 | | | | | | | | | |
| RSAD2, IFI27, OAS1, IFIT1, | 0.858 | 0.847 | 0.869 | 0.933 | 0.885 | 0.982 | 0.921 | 0.839 | 1 |
| ISG15, HERC6, SLC1A2, IL1R2, | | | | | | | | | |
| OLAH, FAM20A, RETN, MMP8 | | | | | | | | | |
| IFI27, OAS1, IFIT1, IFI44L, | 0.858 | 0.846 | 0.869 | 0.935 | 0.886 | 0.983 | 0.922 | 0.840 | 1 |
| ISG15, HERC6, SLC1A2, IL1R2, | | | | | | | | | |
| OLAH, FAM20A, RETN, MMP8 | | | | | | | | | |
| RSAD2, IFI27, OAS1, IFIT1, | 0.858 | 0.845 | 0.870 | 0.934 | 0.886 | 0.983 | 0.925 | 0.848 | 1 |
| IFI44L, ISG15, HERC6, SLC1A2, | | | | | | | | | |
| IL1R2, OLAH, FAM20A, MMP8 | | | | | | | | | |
| RSAD2, IFI27, OAS1, IFI44L, | 0.858 | 0.845 | 0.871 | 0.941 | 0.896 | 0.987 | 0.943 | 0.877 | 1 |
| SIGLEC1, ISG15, HERC6, | | | | | | | | | |
| SLC1A2, OLAH, FAM20A, | | | | | | | | | |
| RETN, MMP8 | | | | | | | | | |
| RSAD2, IFI27, OAS1, IFIT1, | 0.857 | 0.845 | 0.870 | 0.937 | 0.888 | 0.986 | 0.947 | 0.883 | 1 |
| IFI44L, SIGLEC1, ISG15, | | | | | | | | | |
| SLC1A2, OLAH, FAM20A, | | | | | | | | | |
| RETN, MMP8 | | | | | | | | | |
| RSAD2, IFI27, OAS1, IFIT1, | 0.857 | 0.847 | 0.868 | 0.937 | 0.892 | 0.981 | 0.938 | 0.866 | 1 |
| IFI44L, SIGLEC1, ISG15, | | | | | | | | | |
| HERC6, SLC1A2, IL1R2, | | | | | | | | | |
| FAM20A, RETN | | | | | | | | | |
| RSAD2, IFI27, OAS1, IFIT1, | 0.857 | 0.846 | 0.868 | 0.934 | 0.884 | 0.984 | 0.941 | 0.872 | 1 |
| IFI44L, SIGLEC1, ISG15, | | | | | | | | | |
| HERC6, SLC1A2, FAM20A, | | | | | | | | | |
| RETN, MMP8 | | | | | | | | | |
| RSAD2, IFI27, OAS1, IFIT1, | 0.857 | 0.845 | 0.869 | 0.934 | 0.885 | 0.984 | 0.923 | 0.842 | 1 |
| IFI44L, ISG15, HERC6, SLC1A2, | | | | | | | | | |
| OLAH, FAM20A, RETN, MMP8 | | | | | | | | | |
| RSAD2, IFI27, OAS1, IFI44L, | 0.856 | 0.844 | 0.869 | 0.936 | 0.889 | 0.984 | 0.924 | 0.843 | 1 |
| ISG15, HERC6, SLC1A2, IL1R2, | | | | | | | | | |
| OLAH, FAM20A, RETN, MMP8 | | | | | | | | | |
| RSAD2, IFI27, OAS1, IFIT1, | 0.856 | 0.845 | 0.867 | 0.938 | 0.894 | 0.982 | 0.949 | 0.879 | 1 |
| IFI44L, SIGLEC1, ISG15, | | | | | | | | | |
| SLC1A2, IL1R2, FAM20A, | | | | | | | | | |
| RETN, MMP8 | | | | | | | | | |
| RSAD2, IFI27, OAS1, IFIT1, | 0.856 | 0.844 | 0.868 | 0.937 | 0.890 | 0.985 | 0.924 | 0.844 | 1 |
| IFI44L, HERC6, SLC1A2, IL1R2, | | | | | | | | | |
| OLAH, FAM20A, RETN, MMP8 | | | | | | | | | |
| RSAD2, IFI27, OAS1, IFIT1, | 0.856 | 0.843 | 0.869 | 0.937 | 0.889 | 0.984 | 0.950 | 0.886 | 1 |
| IFI44L, SIGLEC1, SLC1A2, | | | | | | | | | |
| IL1R2, OLAH, FAM20A, RETN, | | | | | | | | | |
| MMP8 | | | | | | | | | |
| RSAD2, IFI27, OAS1, IFIT1, | 0.856 | 0.844 | 0.867 | 0.934 | 0.884 | 0.983 | 0.923 | 0.843 | 1 |
| IFI44L, ISG15, SLC1A2, IL1R2, | | | | | | | | | |
| OLAH, FAM20A, RETN, MMP8 | | | | | | | | | |
| RSAD2, IFI27, IFIT1, IFI44L, | 0.855 | 0.843 | 0.867 | 0.935 | 0.888 | 0.983 | 0.923 | 0.844 | 1 |
| ISG15, HERC6, SLC1A2, IL1R2, | | | | | | | | | |
| OLAH, FAM20A, RETN, MMP8 | | | | | | | | | |
| RSAD2, OAS1, IFIT1, IFI44L, | 0.854 | 0.844 | 0.865 | 0.924 | 0.869 | 0.980 | 0.962 | 0.907 | 1 |
| SIGLEC1, ISG15, HERC6, IL1R2, | | | | | | | | | |
| OLAH, FAM20A, RETN, MMP8 | | | | | | | | | |
| RSAD2, IFI27, OAS1, IFIT1, | 0.854 | 0.842 | 0.865 | 0.934 | 0.886 | 0.982 | 0.920 | 0.835 | 1 |
| IFI44L, ISG15, HERC6, SLC1A2, | | | | | | | | | |
| IL1R2, OLAH, FAM20A, RETN | | | | | | | | | |
| RSAD2, IFI27, OAS1, IFIT1, | 0.853 | 0.843 | 0.864 | 0.932 | 0.883 | 0.981 | 0.949 | 0.890 | 1 |
| IFI44L, SIGLEC1, HERC6, | | | | | | | | | |
| SLC1A2, IL1R2, OLAH, RETN, | | | | | | | | | |
| MMP8 | | | | | | | | | |
| RSAD2, OAS1, IFIT1, IFI44L, | 0.853 | 0.842 | 0.863 | 0.929 | 0.882 | 0.976 | 0.954 | 0.890 | 1 |
| SIGLEC1, ISG15, HERC6, | | | | | | | | | |
| SLC1A2, IL1R2, OLAH, | | | | | | | | | |
| FAM20A, MMP8 | | | | | | | | | |
| RSAD2, IFI27, OAS1, IFIT1, | 0.852 | 0.842 | 0.862 | 0.931 | 0.881 | 0.981 | 0.943 | 0.881 | 1 |
| IFI44L, SIGLEC1, ISG15, | | | | | | | | | |
| HERC6, SLC1A2, IL1R2, OLAH, | | | | | | | | | |
| RETN | | | | | | | | | |
| OAS1, IFIT1, IFI44L, SIGLEC1, | 0.852 | 0.841 | 0.862 | 0.931 | 0.884 | 0.977 | 0.950 | 0.886 | 1 |
| ISG15, HERC6, SLC1A2, IL1R2, | | | | | | | | | |
| OLAH, FAM20A, RETN, MMP8 | | | | | | | | | |
| IFI27, OAS1, IFIT1, IFI44L, | 0.851 | 0.840 | 0.863 | 0.932 | 0.883 | 0.980 | 0.947 | 0.887 | 1 |
| SIGLEC1, ISG15, HERC6, | | | | | | | | | |
| SLC1A2, IL1R2, OLAH, RETN, | | | | | | | | | |
| MMP8 | | | | | | | | | |
| RSAD2, OAS1, IFIT1, IFI44L, | 0.851 | 0.841 | 0.862 | 0.929 | 0.880 | 0.978 | 0.942 | 0.877 | 1 |
| SIGLEC1, ISG15, HERC6, | | | | | | | | | |
| SLC1A2, OLAH, FAM20A, | | | | | | | | | |
| RETN, MMP8 | | | | | | | | | |
| RSAD2, OAS1, IFIT1, SIGLEC1, | 0.851 | 0.841 | 0.861 | 0.930 | 0.883 | 0.976 | 0.958 | 0.894 | 1 |
| ISG15, HERC6, SLC1A2, IL1R2, | | | | | | | | | |
| OLAH, FAM20A, RETN, MMP8 | | | | | | | | | |
| RSAD2, IFI27, OAS1, IFIT1, | 0.851 | 0.839 | 0.863 | 0.932 | 0.882 | 0.981 | 0.949 | 0.890 | 1 |
| SIGLEC1, ISG15, HERC6, | | | | | | | | | |
| SLC1A2, IL1R2, OLAH, RETN, | | | | | | | | | |
| MMP8 | | | | | | | | | |
| RSAD2, IFIT1, IFI44L, SIGLEC1, | 0.850 | 0.840 | 0.860 | 0.931 | 0.885 | 0.978 | 0.954 | 0.892 | 1 |
| ISG15, HERC6, SLC1A2, IL1R2, | | | | | | | | | |
| OLAH, FAM20A, RETN, MMP8 | | | | | | | | | |
| RSAD2, IFI27, OAS1, IFIT1, | 0.850 | 0.839 | 0.861 | 0.932 | 0.883 | 0.981 | 0.950 | 0.892 | 1 |
| IFI44L, SIGLEC1, ISG15, | | | | | | | | | |
| HERC6, SLC1A2, OLAH, RETN, | | | | | | | | | |
| MMP8 | | | | | | | | | |

Performance obtained for the identification of the viral or bacterial nature of the infection on the basis of the target 13-gene combinations:

TABLE 17

| | AUC | | | AUC | CI 95% | | AUC | CI 95% | |
|---|---|---|---|---|---|---|---|---|---|
| Model | model | CI 95% | | Train | Train | | Test | Test | |
| RSAD2, IFI27, OAS1, IFIT1, | 0.857 | 0.845 | 0.870 | 0.938 | 0.887 | 0.989 | 0.950 | 0.886 | 1 |
| IFI44L, SIGLEC1, ISG15, | | | | | | | | | |
| HERC6, IL1R2, OLAH, | | | | | | | | | |
| FAM20A, RETN, MMP8 | | | | | | | | | |
| RSAD2, IFI27, OAS1, IFIT1, | 0.856 | 0.844 | 0.868 | 0.943 | 0.900 | 0.986 | 0.946 | 0.877 | 1 |
| IFI44L, SIGLEC1, ISG15, | | | | | | | | | |
| HERC6, SLC1A2, IL1R2, OLAH, | | | | | | | | | |
| FAM20A, MMP8 | | | | | | | | | |
| RSAD2, IFI27, OAS1, IFIT1, | 0.853 | 0.840 | 0.866 | 0.944 | 0.900 | 0.987 | 0.945 | 0.876 | 1 |
| SIGLEC1, ISG15, HERC6, | | | | | | | | | |
| SLC1A2, IL1R2, OLAH, | | | | | | | | | |
| FAM20A, RETN, MMP8 | | | | | | | | | |
| IFI27, OAS1, IFIT1, IFI44L, | 0.853 | 0.839 | 0.866 | 0.942 | 0.899 | 0.986 | 0.943 | 0.873 | 1 |
| SIGLEC1, ISG15, HERC6, | | | | | | | | | |
| SLC1A2, IL1R2, OLAH, | | | | | | | | | |
| FAM20A, RETN, MMP8 | | | | | | | | | |
| RSAD2, IFI27, OAS1, IFIT1, | 0.852 | 0.838 | 0.865 | 0.942 | 0.898 | 0.986 | 0.943 | 0.877 | 1 |
| IFI44L, SIGLEC1, HERC6, | | | | | | | | | |
| SLC1A2, IL1R2, OLAH, | | | | | | | | | |
| FAM20A, RETN, MMP8 | | | | | | | | | |
| RSAD2, IFI27, OAS1, IFIT1, | 0.851 | 0.840 | 0.863 | 0.940 | 0.897 | 0.983 | 0.949 | 0.877 | 1 |
| IFI44L, SIGLEC1, ISG15, | | | | | | | | | |
| HERC6, SLC1A2, IL1R2, | | | | | | | | | |
| FAM20A, RETN, MMP8 | | | | | | | | | |
| RSAD2, IFI27, IFIT1, IFI44L, | 0.851 | 0.838 | 0.864 | 0.945 | 0.903 | 0.986 | 0.943 | 0.874 | 1 |
| SIGLEC1, ISG15, HERC6, | | | | | | | | | |
| SLC1A2, IL1R2, OLAH, | | | | | | | | | |
| FAM20A, RETN, MMP8 | | | | | | | | | |
| RSAD2, IFI27, OAS1, IFIT1, | 0.851 | 0.839 | 0.863 | 0.939 | 0.894 | 0.983 | 0.936 | 0.867 | 1 |
| IFI44L, SIGLEC1, ISG15, | | | | | | | | | |
| HERC6, SLC1A2, IL1R2, OLAH, | | | | | | | | | |
| FAM20A, RETN | | | | | | | | | |

Performance obtained for the identification of the viral or bacterial nature of the infection on the basis of the target 14-gene combinations:

TABLE 18

| | AUC | | | AUC | CI 95% | | AUC | CI 95% | |
|---|---|---|---|---|---|---|---|---|---|
| Model | (model) | CI 95% | | (Train) | (Train) | | (Test) | (Test) | |
| SIGLEC1 ISG15 | 0.84 | 0.83 | 0.86 | 0.94 | 0.90 | 0.99 | 0.94 | 0.87 | 1.00 |
| HERC6 SLC1A2 IL1R2 | | | | | | | | | |
| FAM20A OLAH RETN | | | | | | | | | |
| MMP8 RSAD2, IFI27, | | | | | | | | | |
| OAS1, IFIT1, IFI44L | | | | | | | | | |

Performance obtained for the identification of the viral or bacterial nature of the infection on the basis of the combinations of the 14 target genes and the 4 additional genes:

TABLE 19

| | AUC | | | AUC | CI 95% | | AUC | CI 95% | |
|---|---|---|---|---|---|---|---|---|---|
| Model | (model) | CI 95% | | (Train) | (Train) | | (Test) | (Test) | |
| SIGLEC1 ISG15 | 0.814 | 0.797 | 0.831 | 0.959 | 0.925 | 0.993 | 0.928 | 0.861 | 0.995 |
| HERC6 SLC1A2 IL1R2 | | | | | | | | | |
| FAM20A OLAH RETN | | | | | | | | | |
| MMP8 RSAD2, IFI27, | | | | | | | | | |
| OAS1, IFIT1, IFI44L, | | | | | | | | | |
| PI3, EBI3, ADGRE1 et | | | | | | | | | |
| S100P | | | | | | | | | |

Thus, the transcriptomic signatures according to the invention allow the determination of the viral or bacterial nature of an infection in an infected patient with a robust level of performance, as the AUCs obtained on the TEST set are generally greater than or equal to the AUCs obtained on the TRAIN set.

### BIBLIOGRAPHICAL REFERENCES

Takeuchi O, Akira S (2010), “Pattern recognition receptors and inflammation”; Cell 140 (6): 805-820.
Ramilo O, Mejías A. (2009), “Shifting the paradigm: Host gene signatures for diagnosis of infectious diseases”; Cell Host Microbe 6(3): 199-200.
Fauci, A. S. et al. (2014), “The perpetual challenge of antimicrobial resistance”; JAMA 311, 1853-1854.
Hu et al. (2013), “Gene expression profiles in febrile children with defined viral and bacterial infection”; PNAS 12792-12797, vol. 110, No. 31.
Eric D. Brown et al. (2016), “Antibacterial drug discovery in the resistance era”-Review-doi: 10.1038/nature17042.
Ephraim L. Tsalik et al. (2021), “Discriminating bacterial and viral infection using a rapid host gene expression test”; Critical Care Medicine—Vol. 49, No. 10.
Mark A. Poritz et al. (2011), “FilmArray, an automated Nested Multiplex PCR System for Multi-Pathogen Detection: development and application to respiratory tract infection”, PLOS ONE 6 (10): e26047.
William M. Schneider et al. (2014), “Interferon-stimulated genes: a complex web of host response” Annu. Rev. Immunol. 2014; 32:513-545. doi:10.1146/annurev-immunol-032713-120231.
Emily Yang et al. (2020), “All about the RNA: interferon-stimulated genes that interfere with viral RNA processes”; Front. Immunol. 11:605024.
Trine H. Mogensen et al. (2001), “Molecular pathways in virus-induced cytokine production”; Microbiology and Molecular Biology Review, March 2001, pp. 131-150.
Kawasaki et al. (2014), “Toll-like receptor signaling pathway”; Front. Immunol., Sec. Cancer Immunity and Immunotherapy.
Hochrainer et al. (2005), “The human HERC family of ubiquitin ligases: novel members, genomic organization, expression profiling, and evolutionary aspects”; Genomics 85 153-164

1. An in vitro or ex vivo method for determining the viral or bacterial nature of an infection from a biological sample of a subject who is infected, or who is liable to be infected, comprising the following steps:
(a) measuring the expression of at least one target viral gene and at least one target bacterial gene chosen from the target bacterial genes OLAH, FAM20A, IL1R2, MMP8, RETN and SLC1A2,
(b) comparing the expressions measured in step (a) with predetermined reference expression values of said target genes, and
(c) concluding as to the viral or bacterial nature of the infection on the basis of the comparison results.

2. The method as claimed in claim 1, characterized in that the target viral gene is chosen from the target viral genes of the interferon pathway, the pro-inflammatory cytokine pathway, the Toll-like receptor signaling pathway, the RIG-I-like receptor signaling pathway and the MHC class II-mediated antigen presentation pathway.

3. The method as claimed in claim 1 or 2, characterized in that the target viral gene is chosen from the target viral genes involved in the interferon pathway and the MHC class II-mediated antigen presentation pathway.

4. The method as claimed in claim 3, characterized in that the target viral genes are chosen from IFI27, SIGLEC1, IFN-α, IFN-β, IFN-ε, IFN-κ, IFN-ω, IFN-γ, IFNL1, IFNL2, IFNL3, ADAR1, IFIT1, IFIT2, IFIT3, IFIT5, IFI44L, ISG15, ISG20, MDA5, OAS1, OAS2, OAS3, OASL, PARP12, PKR, RIG-I, RNaseL, RSAD2, RBBP6, SHFL, TRIM22, TRIM25, TRIM32, TRIM69, Viperin, ZAP, ZCCHC3, ZNFX1, HERC1, HERC2, HERC3, HERC4, HERC5, HERC6 and combinations thereof.

5. The method as claimed in one of claims 1 to 4, characterized in that step (a) comprises or consists in measuring at least one target viral gene chosen from IFI27, SIGLEC1, ISG15, HERC6, RSAD2, OAS1, IFIT1 and IFI44L and at least one target bacterial gene chosen from OLAH, FAM20A, IL1R2, MMP8, RETN and SLC1A2.

6. The method as claimed in claim 5, characterized in that step (a) comprises measuring the expression of the target viral gene IFI27 and of the target bacterial gene OLAH.

7. The method as claimed in claim 6, characterized in that step (a) also comprises measuring the expression of the target bacterial gene FAM20A.

8. The method as claimed in one of claims 1 to 5, characterized in that measurement step (a) comprises or consists in measuring the expression of two target viral genes chosen from IFI27, SIGLEC1, ISG15, HERC6, RSAD2, OAS1, IFIT1 and IFI44L and of two target bacterial genes chosen from OLAH, FAM20A, IL1R2, MMP8, RETN and SLC1A2, preferably chosen from OLAH, FAM20A, IL1R2 and MMP8.

9. The method as claimed in one of claims 1 to 5, characterized in that measurement step (a) comprises or consists in measuring the expression of two to five target viral genes chosen from SIGLEC1, ISG15, HERC6, RSAD2, IFI27, OAS1, IFIT1 and IFI44L and of three or four target bacterial genes chosen from OLAH, FAM20A, IL1R2, MMP8, RETN and SLC1A2, preferably chosen from OLAH, FAM20A, IL1R2 and MMP8.

10. The method as claimed in claim 5, characterized in that measurement step (a) comprises or consists in measuring the expression of the target viral gene IFI27, and optionally at least one other viral gene chosen from SIGLEC1, ISG15, HERC6, RSAD2, OAS1, IFIT1 and IFI44L, and of at least two target bacterial genes chosen from OLAH, FAM20A, IL1R2, MMP8, RETN and SLC1A2, preferably chosen from OLAH, FAM20A, IL1R2 and MMP8.

11. The method as claimed in claim 5, characterized in that step (a) comprises or consists in measuring at least one target viral gene chosen from SIGLEC1, ISG15 and HERC6, and at least one target bacterial gene chosen from OLAH, FAM20A, IL1R2, MMP8, RETN and SLC1A2, preferably from IL1R2, OLAH and FAM20A.

12. The method as claimed in claim 5, characterized in that step (a) comprises or consists in measuring at least one target viral gene chosen from IFI27, SIGLEC1, ISG15, HERC6, RSAD2, OAS1, IFIT1 and IFI44L, the target bacterial gene OLAH, and at least one other target bacterial gene chosen from FAM20A, IL1R2, MMP8, RETN and SLC1A2, preferably from FAM20A, IL1R2 and MMP8.

13. The method as claimed in claim 5, characterized in that step (a) comprises or consists in measuring at least one target viral gene chosen from IFI27, SIGLEC1, ISG15, HERC6, RSAD2, OAS1, IFIT1 and IFI44L, the target bacterial gene FAM20A, and at least one other target bacterial gene chosen from OLAH, IL1R2, MMP8, RETN and SLC1A2, preferably from OLAH, IL1R2 and MMP8.

14. The method as claimed in claim 5, characterized in that measurement step (a) comprises or consists in measuring the expression of the target viral gene IFI27, of another target viral gene chosen from HERC6, SIGLEC1, IFI44L and ISG15, of the target bacterial gene FAM20A and of another target bacterial gene chosen from OLAH and IL1R2.

15. The method as claimed in claim 9, characterized in that step (a) comprises or consists in measuring the expression of the target viral gene IFI27, of two other target viral genes chosen from SIGLEC1, HERC6, OAS1 and IFIT1, of the target bacterial gene FAM20A and of another target bacterial gene chosen from OLAH, IL1R2 and MMP8.

16. The method as claimed in claim 5, characterized in that measurement step (a) comprises or consists in measuring the expression of the target viral genes IFI27 and SIGLEC1, of another target viral gene chosen from IFIT1, HERC6, ISG15 and ISG15, of the target bacterial genes FAM20A and MMP8, and of another target bacterial gene chosen from OLAH and IL1R2.

17. The method as claimed in claim 5, characterized in that measurement step (a) comprises or consists in measuring the expression of the target viral genes IFI27 and SIGLEC1, of two other target viral genes chosen from IFIT1, OAS1, HERC6 and ISG15, of the target bacterial genes FAM20A and MMP8, and of another target bacterial gene chosen from OLAH and IL1R2.

18. The method as claimed in claim 1, characterized in that step (a) consists in measuring a combination of target viral and bacterial genes chosen from the combinations of target genes listed in Tables 6 to 12 and which have an AUC of the model for determining the viral or bacterial nature of the infection of at least 0.90.

19. The method as claimed in one of claims 5 to 18, characterized in that the predetermined reference expression value of the target genes corresponds to the respective expression of said target genes in a reference biological sample obtained from a subject having a bacterial infection, and in that it is concluded that the infection is viral in nature when the results of comparison of the expression of the target genes reveal at least one variation in expression chosen from:
an over-expression of SIGLEC1,
an over-expression of ISG15,
an over-expression of HERC6,
an over-expression of RSAD2,
an over-expression of IFI27,
an over-expression of OAS1,
an over-expression of IFIT1, and
an over-expression of IFI44L,
and at least one other expression variation chosen from:
an under-expression of SLC1A2,
an under-expression of IL1R2,
an under-expression of FAM20A,
an under-expression of OLAH,
an under-expression of RETN, and
an under-expression of MMP8.

20. The method as claimed in one of claims 5 to 18, characterized in that the predetermined reference expression value of said target genes corresponds to the respective expression of said target genes in a reference biological sample obtained from a subject having a viral infection, and in that it is concluded that the infection is bacterial in nature when the results of comparison of the expression of the target genes reveal at least one variation in expression chosen from:
an under-expression of SIGLEC1,
an under-expression of ISG15,
an under-expression of HERC6,
an under-expression of RSAD2,
an under-expression of IFI27,
an under-expression of OAS1,
an under-expression of IFIT1, and
an under-expression of IFI44L,
and at least one other expression variation chosen from:
an over-expression of SLC1A2,
an over-expression of IL1R2,
an over-expression of FAM20A,
an over-expression of OLAH,
an over-expression of RETN, and
an over-expression of MMP8.

21. The method as claimed in one of claims 1 to 20, characterized in that it also comprises a step (a′) of measuring the expression of at least one additional target gene chosen from PI3, EBI3, ADGRE1 and S100P, a step (b′) of comparing the expressions measured in step (a′) with reference expression values of said additional target genes.

22. The method as claimed in one of claims 1 to 21, characterized in that the expression of the target genes is measured at the mRNA level.

23. The method as claimed in one of claims 1 to 22, characterized in that the expression variation is measured by amplification via RT-PCR, preferably quantitative RT-PCR, or nested PCR.

24. The method as claimed in one of claims 1 to 23, characterized in that the expression is normalized relative to the expression of one or more housekeeping genes, preferably chosen from DECR1, HPRT1, PPIB, GAPDH and ACTB.

25. The method as claimed in one of claims 1 to 24, characterized in that the biological sample is taken from a subject who is a child, preferably a child less than 4 years old, and more preferably a child less than 2 years old.

26. The method as claimed in one of claims 1 to 25, characterized in that the biological sample is a blood sample, preferably a whole blood sample.

27. A kit for in vitro or ex vivo measurement of gene expression in a biological sample, said kit comprising means for determining the variation in the level of expression of at least one target viral gene chosen from IFI27, SIGLEC1, ISG15, HERC6, RSAD2, OAS1, IFIT1, IFI44L, and of at least one target bacterial gene chosen from OLAH, FAM20A, IL1R2, MMP8, RETN and SLC1A2, said means preferably being primers or probes.

28. Use of the kit as claimed in claim 27, for determining the viral or bacterial nature of an infection from a biological sample of a subject.
